(19) United States
(12) Patent Application Publication
(10) Pub. No.: US 2025/0215029 A1
(43) Pub. Date: Jul. 3, 2025

(54) STAT MODULATORS AND USES THEREOF

(71) Applicant: Recludix Pharma, Inc., San Diego, CA (US)

(72) Inventors: Howard Bregman, Melrose, MA (US); Neil Bifulco, Sudbury, MA (US); Giovanni Cianchetta, Boxford, MA (US); Brian Hodous, Arlington, MA (US); Samuel K. Reznik, Cambridge, MA (US); Ernest Allen Sickmier, Needham, MA (US); Yong Tang, West Roxbury, MA (US); Andrew Tasker, Simi Valley, CA (US); Xia Tian, Cambridge, MA (US); Rishi G. Vaswani, Lexington, MA (US); John Yeoman, Medford, MA (US)

(21) Appl. No.: 18/726,325

(22) PCT Filed: Jan. 10, 2023
(86) PCT No.: PCT/US2023/010447

§ 371 Date: Jul. 2, 2024

Related U.S. Application Data
(60) Provisional application No. 63/337,425, filed on May 2, 2022.
(60) Provisional application No. 63/297,874, filed on Jan. 10, 2022.

(51) Int. Cl. C07F 9/6561; A61K 31/675

(52) U.S. Cl. CPC C07F 9/6561; A61K 31/675

(57) ABSTRACT

Provided are compounds of Formula (I): and pharmaceutically acceptable salts and compositions thereof, which are useful for treating a variety of conditions associated with STAT3 and/or STAT6.

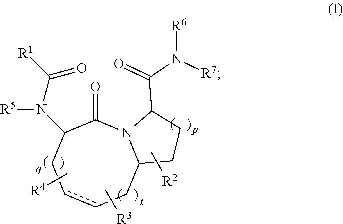

### RELATED APPLICATIONS

This application claims the benefit of priority to U.S. Provisional Application No. 63/297,874, filed Jan. 10, 2022 and U.S. Provisional Application No. 63/337,425, filed May 2, 2022, the entire contents of each of which are incorporated herein by reference.

### BACKGROUND

The Signal Transducer and Activator of Transcription (STAT) family of proteins consists of transcription factors that play an essential role in the regulation of cell processes, such as proliferation, differentiation, apoptosis and angiogenesis. Seven STAT genes have been identified in the human genome: STAT1, STAT2, STAT3, STAT4, STAT5a, STAT5b, and STAT6.

STAT3 has received particular attention because it is strongly associated with the promotion of tumor growth and immune evasion, and the only STAT family member whose genetic deletion results in embryonic lethality. Indeed, aberrantly elevated STAT3 activity has been estimated to occur in more than 70% of human cancers. Activated STAT3 mediates critical gene expression changes and molecular events that dysregulate cell growth and apoptosis, promote angiogenesis, invasion, metastasis, and the development of resistance to apoptosis, and suppress the host's immune surveillance of the tumor, thereby making constitutively-active STAT3 a critical mediator of carcinogenesis and tumor progression.

Another STAT protein that has gained recent interest is STAT6. Recent studies have shown that STAT6 signaling is essential for IL-4- and IL-13-induced epithelial mesenchymal transition (EMT) and aggressiveness of colorectal cancer cells (CRC) cells. STAT6 is involved in several aspects of inflammatory disease and other related conditions.

Given their role in the regulation of cell processes, modulating the activity of one or more STAT proteins, particularly STAT3 and/or STAT6, represent a pivotal area of investigation for the treatment of cancer, inflammatory conditions, and other therapeutic needs.

### SUMMARY

Provided herein are modulators of STAT3 and/or STAT6. Such modulators include those having the structural Formula I:

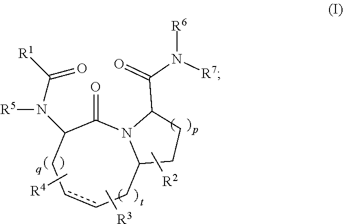

and pharmaceutically acceptable salts and compositions thereof, wherein R¹, R², R³, R⁴, R⁵, R⁶, R⁷, q, t and p are as described herein.

In one aspect, the disclosed compounds of Formula I and pharmaceutically acceptable salts thereof inhibit STAT3 and/or STAT6, and are useful in a variety of therapeutic applications such as, for example, in treating cancer and inflammatory conditions.

Pharmaceutical compositions comprising the compounds and pharmaceutically acceptable salts of the disclosed compounds of Formula I, as well as methods for their preparation are also included.

Methods of treating conditions responsive to the modulation of STAT3 and/or STAT6 using the disclosed compounds, pharmaceutically acceptable salts, and compositions thereof are also included.

### DETAILED DESCRIPTION

### 1. General Description of Compounds

In a first embodiment, provided herein is a compound of structural Formula I:

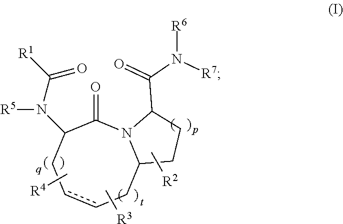

or a pharmaceutically acceptable salt thereof, wherein:
q is 0 or 1 and t is 0, 1, or 2, provided that at least one of q or t is 1;
p is 1 or 2;
the dotted line represents a single or double bond;
R¹ is selected from an 8- to 10-membered fused bicyclic heteroaryl substituted with —CR^{1a}R^{2a}P(O)OR^{1b}OR^{2b} or —CR^{1a}R^{2a}P(O)[OR^{1b}][NH(AA)C(O)OR^{T}], an 8- to 10-membered fused bicyclic heterocyclyl substituted with —CR^{1a}R^{2a}P(O)OR^{1b}OR^{2b} or —CR^{1a}R^{2a}P(O)[OR^{1b}][NH(AA)C(O)OR^{T}], an aryl substituted with CR^{1a}R^{2a}P(O)OR^{1b}OR^{2b} or —CR^{1a}R^{2a}P(O)[OR^{1b}][NH(AA)C(O)OR^{T}], a —(C₁-C₄)alkyl(aryl) wherein said aryl portion of —(C₁-C₄)alkyl(aryl) is substituted with —CR^{1a}R^{2a}P(O)OR^{1b}OR^{2b} or —CR^{1a}R^{2a}P(O)[OR^{1b}][NH(AA)C(O)OR^{T}], and a —(C₂-C₄)alkenyl(aryl) wherein said aryl portion of —(C₂-C₄)alkenyl(aryl) is substituted with —CR^{1a}R^{2a}P(O)OR^{1b}OR^{2b} or —CR^{1a}R^{2a}P(O)[OR^{1b}][NH(AA)C(O)OR^{T}];
R^{1a} and R^{2a} are each absent or are independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each absent or independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]-OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]2, 5- to 6-membered heteroaryl, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
or R³ and R⁴ are taken together on the same carbon atom to form a (C₃-C₆)cycloalkyl or a 4- to 6-membered heterocyclyl each optionally substituted with, as valency permits, 1 to 3 groups selected from halo, (C₁-C₄)alkyl, halo(C₁-C₄)alkyl, (C₁-C₄)alkoxy, and halo(C₁-C₄)alkoxy;
R⁵ and R⁶ are each independently selected from hydrogen and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
AA is the residue of an alpha or beta natural or non-natural amino acid;
R^{T} is selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 6-membered heterocyclyl, 5- to 10-membered monocyclic or bicyclic heteroaryl, (C₃-C₆)cycloalkyl, oxo, imino, —O(phenyl), —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 6-membered heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{J} and R^{M} are each independently selected from halo, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, R^{X}, and R^{Z} are each independently selected from halo, cyano, (C₁-C₄)alkyl, halo(C₁-C₄)alkyl, —(C₁-C₄)alkyl(C₁-C₄)alkoxy, hydroxy(C₁-C₄)alkyl, —(C₁-C₄)alkylphenyl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, hydroxyl, oxo, imino, phenyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —C(O)R^{g}, —C(O)NR^{c}R^{d}, and —NR^{a}R^{b}, wherein said phenyl and said phenyl for the group —(C₁-C₄)alkylphenyl are each optionally and independently substituted with, as valency permits 1 to 3 groups selected from halo, cyano, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independently optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

### 2. Definitions

When used in connection to describe a chemical group that may have multiple points of attachment, a hyphen (-) designates the point of attachment of that group to the variable to which it is defined. For example, —NR^{c}C(O)R^{e} means that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

Unless otherwise specified, the term “alkyl” when used alone or as part of a larger moiety, such as “haloalkyl”, and the like, means saturated straight-chain or branched monovalent hydrocarbon radical.

The term “haloalkyl” includes mono, poly, and perhaloalkyl groups where the halogens are independently selected from fluorine, chlorine, bromine, and iodine.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

The term “oxo” means the group ═O.

The term “imino” means the group ═NH.

Unless otherwise specified, the term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, benzothiopheneyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

Unless otherwise specified, the term “heterocyclyl” means a 4- to 12-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”, “heterocyclic moiety”, and “heterocyclic radical”, are used interchangeably herein. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, terahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3′-pyrrolidine]-yl, thiochromanyl, and the like. It will be understood that when specified, optional substituents on a heterocyclyl group may be present on any substitutable position and, include, e.g., the position at which the heterocyclyl is attached (where valency permits).

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “aryl” refers to an aromatic carbocyclic single ring or two fused ring system containing 6 to 10 carbon atoms. Examples include phenyl, indanyl, tetrahydronaphthalene, and naphthyl. In one aspect, the aryl is phenyl or naphthyl.

The terms “cycloalkyl”, used alone or as part of a larger moiety, refers to a saturated cyclic aliphatic monocyclic or bicyclic ring system, as described herein, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The “residue of an amino acid” is the moiety remaining after formation of a bond between a reactive group in another compound (e.g., an amino group) and the carboxylic acid in the amino acid, after formation of a bond between a reactive group in another compound (e.g., a carboxylic acid) and the amino group in the amino acid, or both. As a consequence of the bond(s) formation, the carboxylic acid in the amino acid no longer has the OH group and instead has a bond between the carbonyl group and the reactive group in the compound; the amino group has only one hydrogen atom and instead has a bond between the reactive group in the other compound and the nitrogen of the amino group; or both. For example, the “residue of an alpha amino acid” can be depicted structurally as NH₂CR′R—C(O)—, —NHCR′R—C(O)OH or —NHCR′R—C(O)—; and the “residue of an beta amino acid” can be depicted structurally as or NH₂CR′RCH₂—C(O)—, —NHCR′RCH₂—C(O)OH or —NHCR′RCH₂—C(O)—, where R′ is H or C₁-C₆ alkyl and R is H or C₁-C₆ alkyl optionally substituted with 1 to 3 groups selected from halo, (C₁-C₃)alkoxy, OH, NH₂, —NH(C₁-C₄ alkyl), —N[(C₁-C₄ alkyl)]2, SH, S(C₁-C₄ alkyl), imino, COOH, —COO(C₁-C₄ alkyl), —CO(C₁-C₄ alkyl), —CONH(C₁-C₄ alkyl)phenyl, phenyl, and 5- to 10-membered heteroaryl, wherein said C₁-C₆ alkyl may also be optionally interrupted by a sulfur or nitrogen heteroatom and wherein said phenyl is optionally substituted with 1 to 3 groups selected from OH, cyano, (C₁-C₄ alkyl), and halo(C₁-C₄ alkyl); or R is taken together with the nitrogen atoms from the alpha or beta amino acid residue to form a 4- to 6-membered heterocyclyl. For naturally occurring alpha amino acid (i.e., amino acids that occur in nature), R′ is H and R is selected from hydrogen, methyl, isopropyl, —CH₂CH(CH₃)₂, —(CH₂)₂SCH₃, —CH(CH₃)(CH₂CH₃), CH₂OH, —CH(OH)(CH₃), CH₂SH, —CH₂C(O)NH₂, —(CH₂)₂C(O)NH₂, benzyl, p-hydroxybenzyl, —CH₂(indolyl), —(CH₂)₄NH₂, —(CH₂)₃NHC(═NH₂)NH₂, —CH₂(imidazolyl), —(CH₂)COOH, and —(CH₂)₂COOH; or R taken together with the nitrogen atom of the alpha or beta amio acid residue forms a pyrrolidinyl ring.

Non-natural amino acids are known in the art and include e.g., alpha-alkyl amino acids (e.g., alpha methyl), alpha-alkylalkoxy amino acids (e.g., alpha —CH₂OCH₃), N-methyl amino acids, homo-amino acids, etc.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to isomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of the ring, whereas “trans” refers to substituents oriented on opposite sides of the ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

In certain instances, compounds were isolated and tested as a 1:1 mixture of diastereomers. In such cases, the relative stereochemistry is denoted by the term “rel-” in the compound name and by the use of flat bonds instead of wedges. For example, ((2-(((3S,6S,9S,10aR)-9-(azetidin-1-yl)-3-(rel-(trans)-3-cyano-4-phenylpyrrolidine-1-carbonyl)-5-oxodecahydropyrrolo[1,2-a]azocin-6-yl)carbamoyl)benzo[b]thiophen-5-yl)difluoromethyl)phosphonic acid, having the structure:

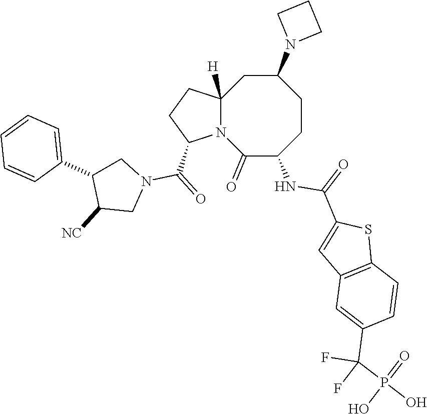 [SMILES: N#C[C@@H]1CN(C(=O)[C@@H]2CC[C@@H]3C[C@@H](N4CCC4)CC[C@H](NC(=O)c4cc5cc(C(F)(F)P(=O)(O)O)ccc5s4)C(=O)N32)C[C@H]1c1ccccc1]

means that the substituents about the pyrrolidine ring are trans and encompass a mixture of both diastereomers

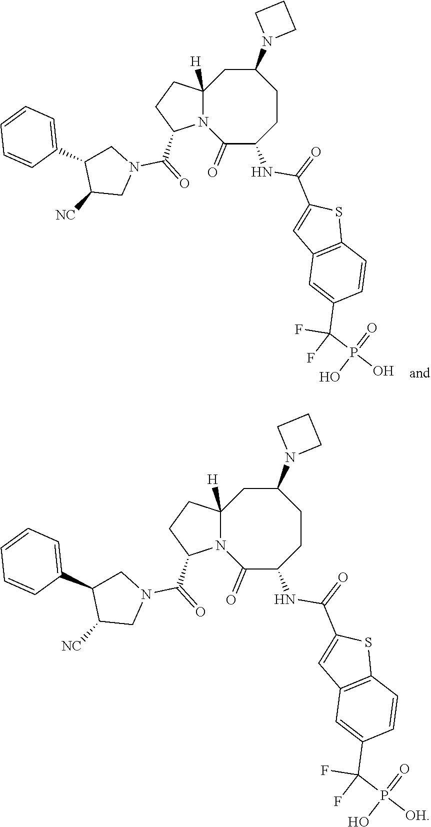 [SMILES: N#C[C@@H]1CN(C(=O)[C@@H]2CC[C@@H]3C[C@@H](N4CCC4)CC[C@H](NC(=O)c4cc5cc(C(F)(F)P(=O)(O)O)ccc5s4)C(=O)N32)C[C@H]1c1ccccc1 ; N#C[C@H]1CN(C(=O)[C@@H]2CC[C@@H]3C[C@@H](N4CCC4)CC[C@H](NC(=O)c4cc5cc(C(F)(F)P(=O)(O)O)ccc5s4)C(=O)N32)C[C@@H]1c1ccccc1]

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that is sufficient to achieve the desired therapeutic effect (such as treatment of a condition recited herein) under the conditions of administration e.g., a dosage of between 0.01-100 mg/kg body weight/day.

### 3. Compounds

In a first embodiment, provided is a compound of structural Formula I:

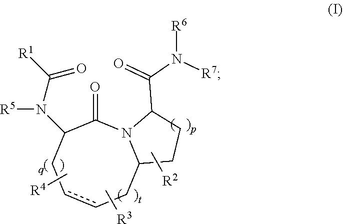

or a pharmaceutically acceptable salt thereof, wherein the variables are as described above.

In a second embodiment, the compound of Formula I is of the structural Formula II:

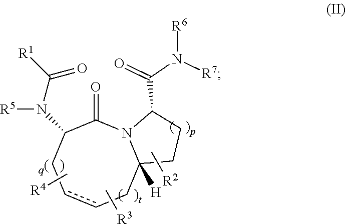

or a pharmaceutically acceptable salt thereof, wherein the variables are as described above for Formula I.

In a third embodiment, the compound of Formula I is of the structural Formula III, IV, V, or VII:

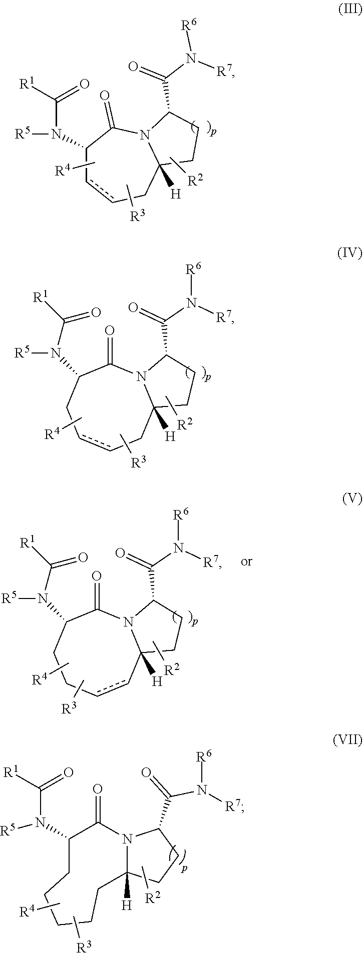

or a pharmaceutically acceptable salt thereof, wherein the variables are as described above for Formula I.

In a fourth embodiment, the compound of Formula I is of the structural Formula VIII, VIII′, IX, X, XI, XII, or XIII:

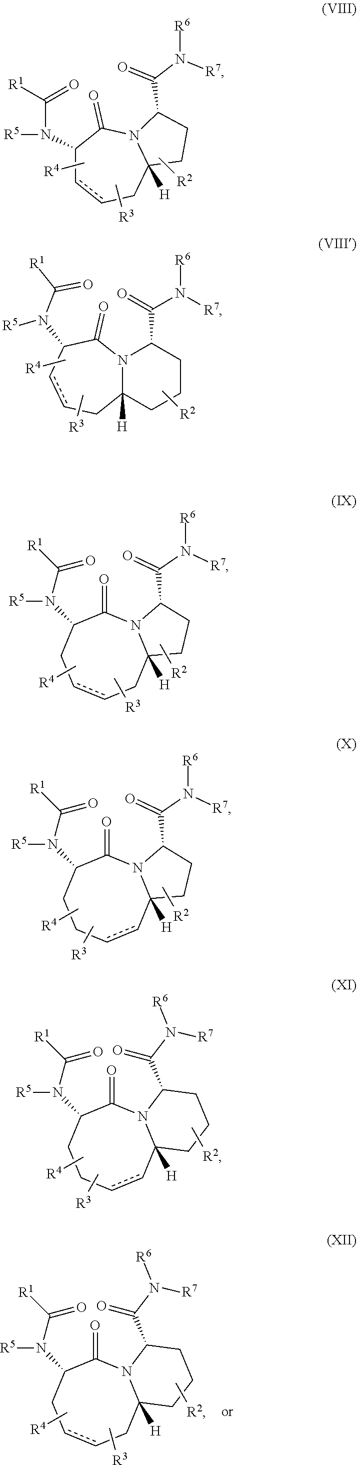

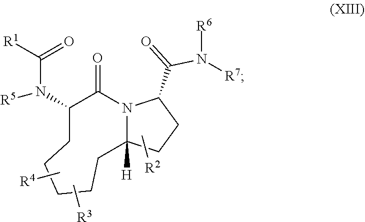

or a pharmaceutically acceptable salt thereof, wherein the variables are as described above for Formula I.

In a sixth embodiment, the compound of Formula I is of the structural Formula XIV, XV, XVI, XVI′, XVII, XVIII, XX, XXI, XXII, XXIII, XXIV, XXV, XXVI, XXVII, or XXVIII:

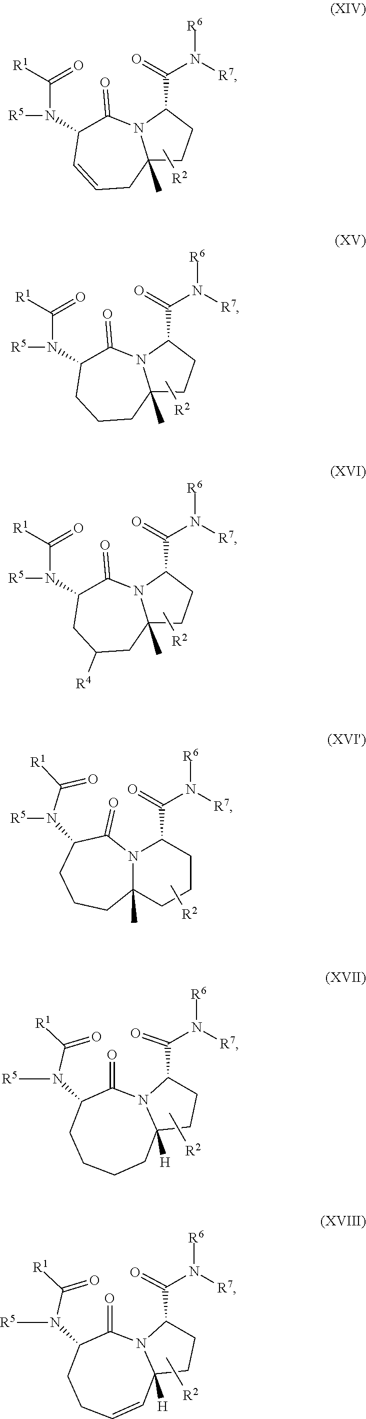

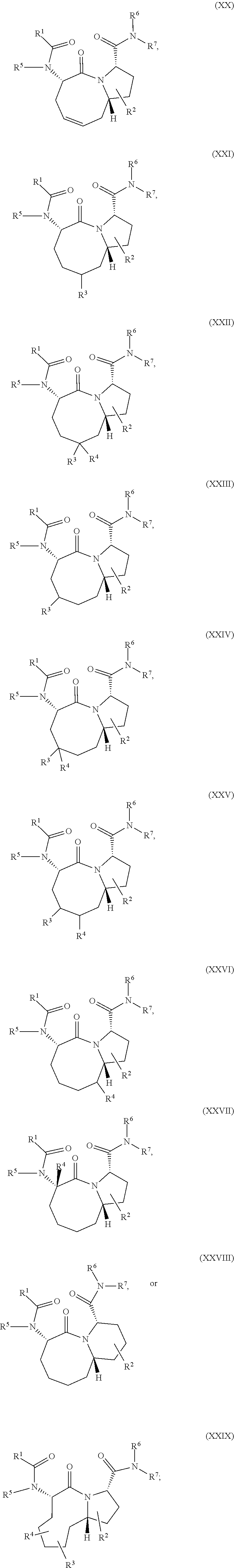

or a pharmaceutically acceptable salt thereof, wherein the variables are as described above for Formula I.

In a seventh embodiment, the compound of Formula I is of the structural Formula XXX, XXXI, XXXII, XXXIII XXXIV, XXXV, XXXVI, or XXVII:

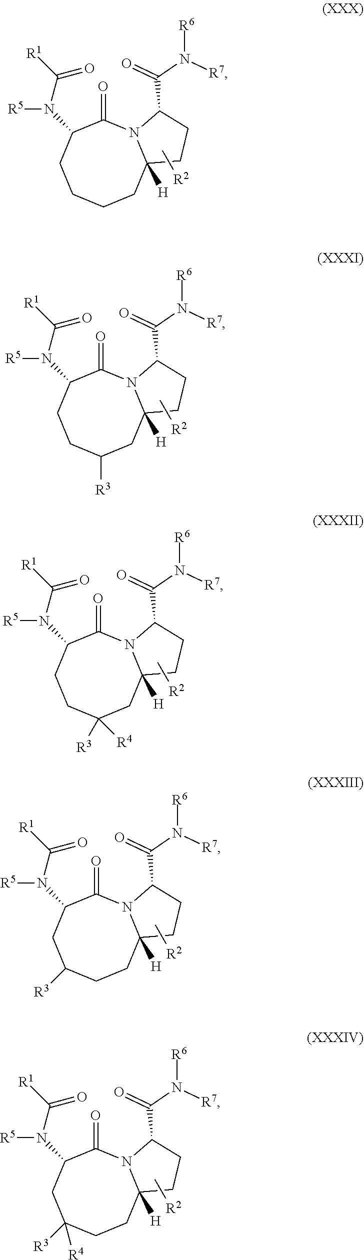

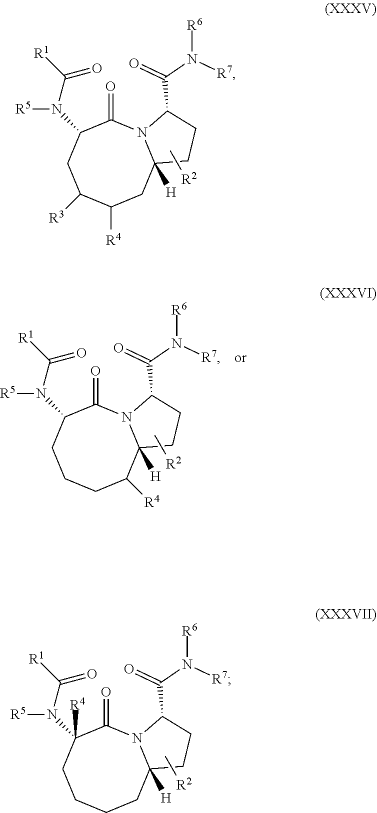

or a pharmaceutically acceptable salt thereof, wherein the variables are as described above for Formula I.

In an eighth embodiment, R³ in the compound of any one of Formulae I, II, III, IV, V, VII, VIII, IX, X, XI, XII, XIII, XXI, XXII, XXIII, XXIV, XXV, XXIX, XXXI, XXXII, XXXIII, XXXIV, and XXXV is selected from hydrogen, (C₁-C₄)alkyl, hydroxyl, (C₁-C₄)alkoxy, —(C₁-C₄)alkylphenyl, and 4- to 6-membered heterocyclyl, wherein the remaining variables are as described above for Formula I. Alternatively, as part of an eighth embodiment, R³ in the compound of any one of Formulae I, II, III, IV, V, VII, VIII, IX, X, XI, XII, XIII, XXI, XXII, XXIII, XXIV, XXV, XXIX, XXXI, XXXII, XXXIII, XXXIV, and XXXV is selected from hydrogen, (C₁-C₂)alkyl, hydroxyl, (C₁-C₂)alkoxy, benzyl, and azetidinyl, wherein the remaining variables are as described above for Formula I.

In a ninth embodiment, R⁴ in the compound of any one of Formulae I, II, III, IV, V, VII, VIII, IX, X, XI, XII, XIII, XVI, XXII, XXIV, XXVI, XXIX, XXXII, XXXIV, XXXV, and XXXVI is selected from hydrogen (C₁-C₄)alkyl, and hydroxyl, wherein the remaining variables are as described above for Formula I.

In a tenth embodiment, R³ and R⁴ in the compound of any one of Formulae I, II, III, IV, V, VII, VIII, IX, X, XI, XII, XIII, XVI, XXI, XXII, XXIII, XXIV, XXV, XXVI, XXIX, XXXI, XXXII, XXXIII, XXXIV, XXXV, and XXXVI are hydrogen, wherein the remaining variables are as described above for Formula I.

In an eleventh embodiment, R³ and R⁴ in the compound of any one of Formulae I, II, III, IV, V, VII, VIII, IX, X, XI, XII, XIII, XVI, XXI, XXII, XXIII, XXIV, XXV, XXVI, XXIX, XXXI, XXXII, XXXIII, XXXIV, XXXV, and XXXVI are taken together on the same carbon atom to form a (C₃-C₆)cycloalkyl, wherein the remaining variables are as described above for Formula I. Alternatively, as part of an eleventh embodiment, R³ and R⁴ in the compound of any one of Formulae I, II, III, IV, V, VII, VIII, IX, X, XI, XII, XIII, XVI, XXI, XXII, XXIII, XXIV, XXV, XXVI, XXIX, XXXI, XXXII, XXXIII, XXXIV, XXXV, and XXXVI are taken together on the same carbon atom to form cyclopropyl, wherein the remaining variables are as described above for Formula I.

In a twelfth embodiment, R² in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from hydrogen and hydroxyl, wherein the remaining variables are as described above for Formula I or any one of the eighth through eleventh embodiments. Alternatively, as part of a twelfth embodiment, R² in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is hydrogen, wherein the remaining variables are as described above for Formula I or any one of the eighth through eleventh embodiments.

In a thirteenth embodiment, R⁵ in the compound of any one of Formulae I, II, III, IV, V, VII, VIII, IX, X, XI, XII, XIII, XIV, XV, XVI, XVI′, XVII, XVIII, VIII′, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is hydrogen, wherein the remaining variables are as described above for Formula I or any one of the eighth through twelfth embodiments.

In a fourteenth embodiment, R¹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from 8- to 10-membered fused bicyclic heteroaryl and aryl, each of which are substituted with —CR^{1a}R^{2a}P(O)OR^{1b}OR^{2b} or —CR^{1a}R^{2a}P(O)[OR^{1b}][NH(AA)C(O)OR^{T}], wherein the remaining variables are as described above for Formula I or any one of the eighth through thirteenth embodiments. Alternatively, as part of a fourteenth embodiment, R¹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from benzothiophenyl, indolyl, and naphthalenyl, each of which are substituted with —CR^{1a}R^{2a}P(O)OR^{1b}OR^{2b} or —CR^{1a}R^{2a}P(O)[OR^{1b}][NH(AA)C(O)OR^{T}], wherein the remaining variables are as described above for Formula I or any one of the eighth through thirteenth embodiments. In another alternative, as part of a fourteenth embodiment, R¹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from

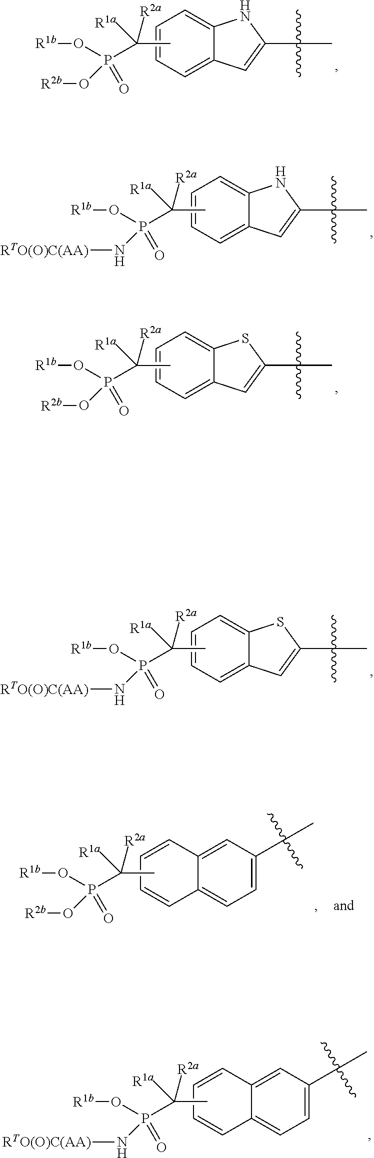

wherein the remaining variables are as described above for Formula I or any one of the eighth through thirteenth embodiments. In yet another alternative, as part of a fourteenth embodiment, R¹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is

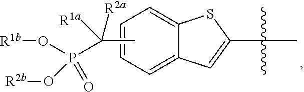

wherein the remaining variables are as described above for Formula I or any one of the eighth through thirteenth embodiments.

In a fifteenth embodiment, R^{1a} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is hydrogen and R^{2a} is fluoro or R^{1a} is fluoro and R^{2a} is fluoro, wherein the remaining variables are as described above for Formula I or any one of the eighth through fourteenth embodiments. Alternatively, as part of a fifteenth embodiment, R^{1a} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is fluoro and R^{2a} is fluoro, wherein the remaining variables are as described above for Formula I or any one of the eighth through fourteenth embodiments.

In a sixteenth embodiment, R^{1b} and R^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII are each independently selected from hydrogen, (C₁-C₄)alkyl, —[(C₁-C₄)alkyl]-OC(O)—[(C₁-C₄)alkyl], —[(C₁-C₄)alkyl]-OC(O)O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, phenyl, pyridinyl, and naphthalenyl, wherein said phenyl, pyridinyl, and naphthalenyl are each optionally and independently substituted with cyano, wherein the remaining variables are as described above for Formula I or any one of the eighth through fifteenth embodiments. Alternatively, as part of a sixteenth embodiment, R^{1b} and R^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII are each —[(C₁-C₄)alkyl]-OC(O)—[(C₁-C₄)alkyl], wherein the remaining variables are as described above for Formula I or any one of the eighth through fifteenth embodiments. In another alternative, as part of a sixteenth embodiment, R^{1b} and R^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII are each hydrogen, wherein the remaining variables are as described above for Formula I or any one of the eighth through fifteenth embodiments.

In a seventeenth embodiment, —CR^{1a}R^{2a}P(O)OR^{1b}OR^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from

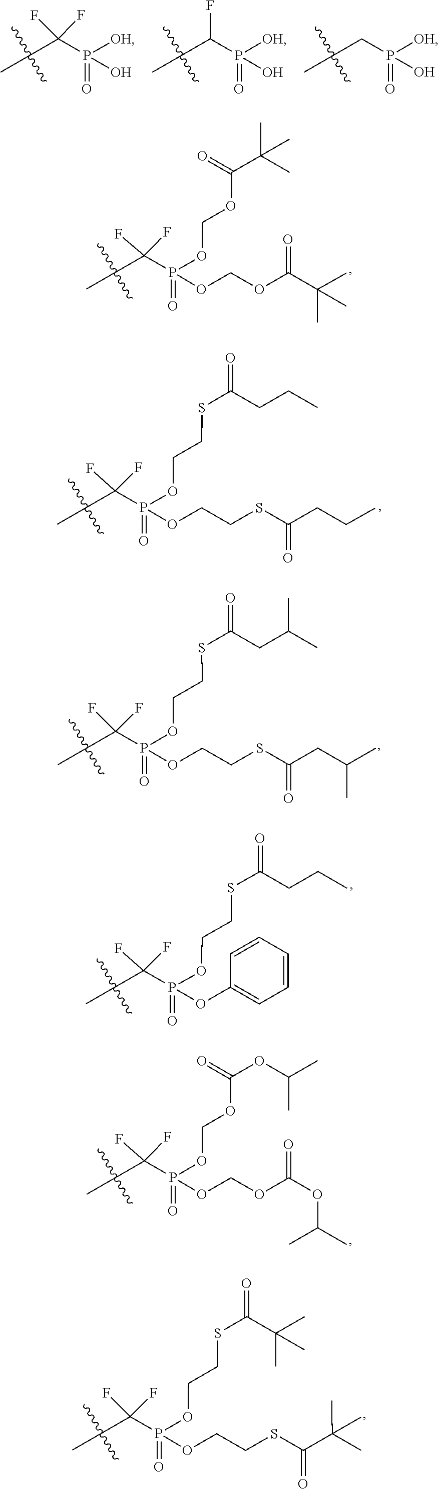

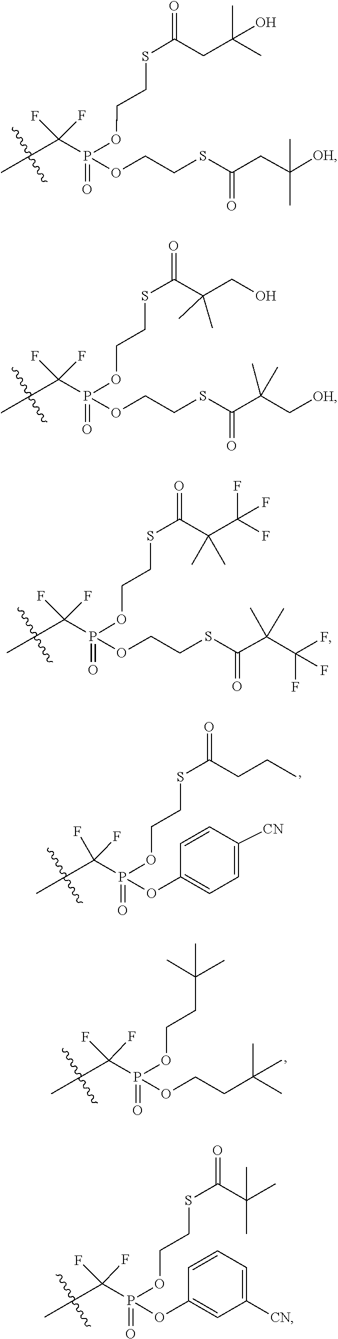

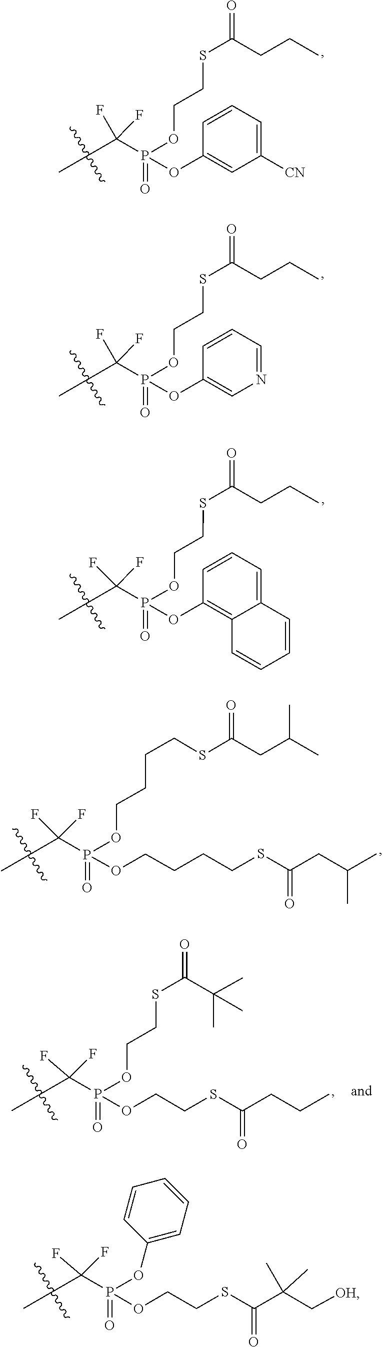

wherein the remaining variables are as described above for Formula I or any one of the eighth through sixteenth embodiments. Alternatively, as part of a seventeenth embodiment, —CR^{1a}R^{2a}P(O)OR^{1b}OR^{2b} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is

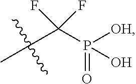 [SMILES: *C(F)(F)P(=O)(O)O]

wherein the remaining variables are as described above for Formula I or any one of the eighth through sixteenth embodiments.

In an eighteenth embodiment, —NH[AA]C(O)OR^{T} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is —NHC(R′)(R)C(O)R^{T} or —NHC(R′)(R)CH₂C(O)R^{T}, wherein R′ is hydrogen, (C₁-C₃)alkyl, or (C₁-C₃)alkyl(C₁-C₃)alkoxy and R is selected from hydrogen, methyl, isopropyl, —CH₂CH(CH₃)₂, —(CH₂)₂SCH₃, —CH(CH₃)(CH₂CH₃), CH₂OH, —CH(OH)(CH₃), CH₂SH, —CH₂C(O)NH₂, —(CH₂)₂C(O)NH₂, benzyl, p-hydroxybenzyl, —CH₂(indolyl), —(CH₂)₄NH₂, —(CH₂)₃NHC(═NH₂)NH₂, —CH₂(imidazolyl), —(CH₂)COOH, and —(CH₂)₂COOH; or R taken together with the nitrogen atom of —NHC(R′)(R)C(O)R^{T} or —NHC(R′)(R)CH₂C(O)R^{T} forms a pyrrolidinyl ring, wherein the remaining variables are as described above for Formula I or any one of the eighth through seventeenth embodiments.

In a nineteenth embodiment, R⁷ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from (C₁-C₄)alkyl, phenyl, 5- or 6-membered monocyclic heterocyclyl, 9- or 10-membered fused bicyclic heterocyclyl, 5- or 6-membered monocyclic heteroaryl, and 9- or 10-membered fused bicyclic heteroaryl, wherein said (C₁-C₄)alkyl is optionally substituted with, as valency permits, 1 to 3 groups selected from R^{Y} and said phenyl, 5- or 6-membered monocyclic heterocyclyl, 9- or 10-membered fused bicyclic heterocyclyl, 5- or 6-membered monocyclic heteroaryl, and 9- or 10-membered fused bicyclic heteroaryl are each optionally substituted with, as valency permits, 1 to 3 groups selected from R^{Z}, and wherein the remaining variables are as described above for Formula I or any one of the eighth through fourteenth and eighteenth embodiments. Alternatively, as part of a nineteenth embodiment, R⁷ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from (C₁-C₄)alkyl, phenyl, pyrrolidinyl, thiochromanyl, dihydrobenzo[b]thiophenyl, pyridinyl, indazolyl, cinnolinyl, and quinolinyl, wherein said (C₁-C₄)alkyl is optionally substituted with, as valency permits, 1 to 3 groups selected from R^{Y} and said phenyl, pyrrolidinyl, thiochromanyl, dihydrobenzo[b]thiophenyl, pyridinyl, indazolyl, cinnolinyl, and quinolinyl are each optionally substituted with, as valency permits, 1 to 3 groups selected from R^{Z}, wherein the remaining variables are as described above for Formula I or any one of the eighth through eighteenth embodiments.

In a twentieth embodiment, R^{Y} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from halo, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X}, wherein the remaining variables are as described above for Formula I or any one of the eighth through nineteenth embodiments. Alternatively, as part of a a twentieth embodiment, R^{Y} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from halo, hydroxyl, 4- to 6-membered heterocyclyl, and 5- to 10-membered monocyclic or bicyclic heteroaryl, wherein said 4- to 6-membered heterocyclyl, and 5- to 10-membered monocyclic or bicyclic heteroaryl are each optionally substituted with, as valency permits, 1 to 3 groups selected from R^{X}, wherein the remaining variables are as described above for Formula I or any one of the eighth through nineteenth embodiments.

In a twenty-first embodiment, R^{Y} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from —C(O)NH₂, —C(O)N(C₁-C₄)alkyl[phenyl]2, hydroxyl, phenyl, imidazolyl, 1,2-dihydropyridinyl, pyridinyl, and pyrazolo[3,4-b]pyridinyl, wherein said phenyl, imidazolyl, 1,2-dihydropyridinyl, pyridinyl, and pyrazolo[3,4-b]pyridinyl are each optionally substituted with, as valency permits, 1 to 3 groups selected from R^{X}, wherein the remaining variables are as described above for Formula I or any one of the eighth through twentieth embodiments. Alternatively, as part of a twenty-first embodiment, R^{Y} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from hydroxyl, imidazolyl, 1,2-dihydropyridinyl, pyridinyl, and pyrazolo[3,4-b]pyridinyl, wherein said phenyl, imidazolyl, 1,2-dihydropyridinyl, pyridinyl, and pyrazolo[3,4-b]pyridinyl are each optionally substituted with, as valency permits, 1 to 3 groups selected from R^{X}, wherein the remaining variables are as described above for Formula I or any one of the eighth through twentieth embodiments.

In a twenty-second embodiment, R^{Z} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from phenyl, —C(O)(C₁-C₄)alkyl, hydroxyl, (C₁-C₄)alkyl, halo, cyano, and (C₁-C₄)alkoxy, wherein the remaining variables are as described above for Formula I or any one of the eighth through twentieth-first embodiments.

In a twenty-third embodiment, R⁶ and R⁷ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII together with the nitrogen atom to which they are attached form 4- to 6-membered monocyclic heterocyclyl, 7- to 13-membered spiro bicyclic heterocyclyl, or 9- to 10-membered fused bicyclic heterocyclyl, each of which being optionally substituted with, as valency permits, 1 to 3 groups selected from R^{Q}, wherein the remaining variables are as described above for Formula I or any one of the eighth through eighteenth embodiments. Alternatively, as part of a twenty-third embodiment, R⁶ and R⁷ in the compound of any one of Formulae XVI′, together with the nitrogen atom to which they are attached form pyrrolidinyl, azetidinyl, piperazinyl, 5-oxa-2,6-diazaspiro[3.4]oct-6-enyl, 6-thia-2,7-diazaspiro[3.4]octanyl, 2-thia-6-azaspiro[3.3]heptanyl, 4-azaspiro[2.4]heptanyl, spiro[indoline-3,3′-pyrrolidinyl], or 1,2,3,4,5,6-hexahydro-2,6-naphthyridinyl, each of which being optionally substituted with, as valency permits, 1 to 3 groups selected from R^{Q}, wherein the remaining variables are as described above for Formula I or any one of the eighth through eighteenth embodiments. In another alternative, as part of a twenty-third embodiment, R⁶ and R⁷ in the compound of any one of Formulae XVI′, together with the nitrogen atom to which they are attached form pyrrolidinyl or azetidinyl, each of which being optionally substituted with, as valency permits, 1 to 3 groups selected from R^{Q}, wherein the remaining variables are as described above for Formula I or any one of the eighth through eighteenth embodiments.

In a twenty-fourth embodiment, R^{Q} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from halo, (C₂-C₄)alkenyl, (C₁-C₄)alkyl, cyano, phenyl, hydroxyl, 4- to 6-membered heterocyclyl, 5- to 10-membered monocyclic or bicyclic heteroaryl, (C₃-C₆)cycloalkyl, oxo, imino, —O(phenyl), —C(O)R^{g}, —NHC(O)R^{e}, —S(O)═NH(C₁-C₄)alkyl, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 4- to 6-membered heterocyclyl, 5- to 10-membered monocyclic or bicyclic heteroaryl, and (C₃-C₆)cycloalkyl are each optionally and independently substituted with, as valency permits, 1 to 3 groups selected from R^{F}, wherein the remaining variables are as described above for Formula I or any one of the eighth through twentieth-third embodiments. Alternatively, as part of a twenty-fourth embodiment, R^{Q} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from halo, (C₂-C₄)alkenyl, (C₁-C₄)alkyl, cyano, phenyl, hydroxyl, morpholinyl, dihydropyridinyl, tetrahydro-2H-thiopyranyl, pyridinyl, pyrrolyl, pyrazolyl, imidazolyl, oxazolyl, triazolyl, indazolyl, benzoimidazolyl, pyrazolo[3,4-b]pyridinyl, cyclohexyl, cyclopropyl, oxo, imino, —O(phenyl), —C(O)R^{g}, —NHC(O)R^{e}, —S(O)═NH(C₁-C₄)alkyl, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, morpholinyl, dihydropyridinyl, tetrahydro-2H-thiopyranyl, pyridinyl, pyrrolyl, pyrazolyl, imidazolyl, oxazolyl, triazolyl, indazolyl, benzoimidazolyl, pyrazolo[3,4-b]pyridinyl, cyclohexyl, and cyclopropyl are each optionally and independently substituted with, as valency permits, 1 to 3 groups selected from R^{F}, wherein the remaining variables are as described above for Formula I or any one of the eighth through twentieth-third embodiments.

In a twenty-fifth embodiment, R^{M} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from 4- to 6-membered heterocyclyl, 5- to 6-membered monocyclic heteroaryl, —S(O)═NH(C₁-C₄)alkyl, cyano, and phenyl, wherein said 4- to 6-membered heterocyclyl, 5- to 6-membered monocyclic heteroaryl, and phenyl are each optionally substituted with, as valency permits, 1 to 3 groups selected from R^{X}, wherein the remaining variables are as described above for Formula I or any one of the eighth through twentieth-fourth embodiments. Alternatively, as part of a twenty-fifth embodiment, R^{M} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from tetrahydropyranyl, pyrazolyl, —S(O)═NH(C₁-C₄)alkyl, cyano, and phenyl, wherein said tetrahydropyranyl, pyrazolyl, and phenyl are each optionally substituted with, as valency permits, 1 to 3 groups selected from R^{X}, wherein the remaining variables are as described above for Formula I or any one of the eighth through twentieth-fourth embodiments.

In a twenty-sixth embodiment, R^{X} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from (C₁-C₄)alkyl, (C₁-C₄)alkoxy, and oxo, wherein the remaining variables are as described above for Formula I or any one of the eighth through twentieth-fifth embodiments.

In a twenty-seventh embodiment, R^{F} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from halo, (C₁-C₄)alkyl, hydroxy(C₁-C₄)alkyl, (C₁-C₄)alkoxy, (C₂-C₄)alkynyl, cyano, oxo, and imino, wherein the remaining variables are as described above for Formula I or any one of the eighth through twentieth-sixth embodiments.

In a twenty-eighth embodiment, R⁹ in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII is selected from (C₁-C₄)alkyl, morpholinyl, imidazolyl, benzyl, and cyclopropyl, wherein the remaining variables are as described above for Formula I or any one of the eighth through twentieth-seventh embodiments.

In a twenty-ninth embodiment, R^{e} and R^{f} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII are each independently selected from (C₁-C₄)alkyl and halo(C₁-C₄)alkyl, wherein the remaining variables are as described above for Formula I or any one of the eighth through twentieth-eighth embodiments.

In a thirtieth embodiment, R′ and R^{d} in the compound of any one of Formulae I, II, III, IV, V, VII, VIII, VIII′, IX, X, XI, XII, XIII, XIV, XV, XVI, XVI′, XVII, XVIII, XX, XXI, XXII, XXIII, XXIV, XXV, XXVI, XXVII, XXVIII, XXIX, XXX, XXXI, XXXII, XXXIII XXXIV, XXXV, XXXVI, or XXXVII are each independently selected from hydrogen and (C₁-C₄)alkyl, wherein the remaining variables are as described above for Formula I or any one of the eighth through tweny-ninth embodiments. Compounds having the Formula I are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of STAT proteins, in particular STAT3 and/or STAT6. In some aspects, the compounds, pharmaceutical acceptable salts, and pharmaceutical compositions described herein inhibit the activity STAT3 and/or STAT6.

In some aspects, the compounds and pharmaceutical compositions described herein are useful in a condition responsive to the modulation of STAT3 and/or STAT6. Thus, provided herein are methods of treating a condition responsive to the modulation (e.g., inhibition) of STAT3 and/or STAT6 in a subject, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a condition responsive to the modulation (e.g., inhibition) of STAT3 and/or STAT6. Also provided is a compound described herein, or a pharmaceutically acceptable salt thereof, or a pharmaceutical composition comprising a disclosed compound or pharmaceutically acceptable salt thereof, for use in treating a condition responsive to the modulation (e.g., inhibition) of STAT3 and/or STAT6.

In one aspect, the condition responsive to the modulation (e.g., inhibition) of STAT3 and/or STAT6 include, but are not limited to, cancer, a neurodegenative disorder, a viral disease, an autoimmune disease, an inflammatory disorder, a hereditary disorder, a hormone-related disease, a metabolic disorder, conditions associated with organ transplantation, immunodeficiency disorders, a destructive bone disorder, a proliferative disorder, an infectious disease, a condition associated with cell death, thrombin-induced platelet aggregation, liver disease, pathologic immune conditions involving T cell activation, a cardiovascular disorder, or a CNS disorder.

In another aspect, the condition responsive to the modulation (e.g., inhibition) of STAT3 and/or STAT6 include, but are not limited to, cancer (see, e.g., Turkson & Jove, Oncogene 2000, 19:6613-6626), diabetes (see. e.g., Gurzov et al., FEBS 2016, 283:3002), cardiovascular disease (see, e.g., Grote et al., Vasc. Pharmacol. 2005, 43:2005), viral disease (see, e.g., Gao et al., J Hepatol. 2012, 57(2):430), autoimmune diseases such as lupus (see, e.g., Goropevsek et al., Clin. Rev. Alleg. & Immun. 2017, 52(2):164), and rheumatoid arthritis (see, e.g., Walker & Smith, J. Rheumat. 2005, 32(9): 1650), autoinflammatory syndromes (see, e.g., Rauch et al., Jak-Stat 2013, 2(1): e23820), atherosclerosis (see, e.g., Ortiz-Munoz et al., Arterio., Thromho., Vase. Bio. 2009, 29:525), psoriasis (see, e.g., Andres et al., Exp. Derm. 2013, 22(5):323), allergic disorders (see, e.g., Oh et al., Eur. Respir. Rev. 2019, 19(115):46), inflammatory bowel disease (see. e.g., Sugimoto, World J Gastroenterol. 2008, 14(33):5110), inflammation (see, e.g., Tamiya et al., Arierio. Thrombo., Vasc. Bio. 2011, 31:980), acute and chronic gout and gouty arthritis, neurological disorders (see, e.g., Campbell, Brain Res. Rev. 2005, 48(2): 166), metabolic syndrome, immunodeficiency disorders such as AIDS and HIV (see, e.g., O'Shea et al., N. Engl. J. Med. 2013, 368:161), destructive bone disorders (see, e.g., Jatiani et al., Genes & Can. 2011, 1(10):979), osteoarthritis, proliferative disorders, Waldenstrom's Macroglobulinemia (see, e.g., Hodge et al., Blood 2014, 123(7):1055) infectious diseases, conditions associated with cell death, pathologic immune conditions involving T cell activation, and CNS disorders.

Proliferative disorders, include, but are not limited to a benign or malignant tumor, solid tumor, liquid tumor, carcinoma of the brain, kidney, liver, adrenal gland, bladder, breast, stomach, gastric tumors, ovaries, colon, rectum, prostate, pancreas, lung, vagina, cervix, testis, genitourinary tract, esophagus, larynx, skin, bone or thyroid, sarcoma, glioblastomas, neuroblastomas, multiple myeloma, gastrointestinal cancer, especially colon carcinoma or colorectal adenoma, a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, Hodgkins and Non-Hodgkins, a mammary carcinoma, follicular carcinoma, undifferentiated carcinoma, papillary carcinoma, seminoma, melanoma, an IL-I driven disorder, an MyD88 driven disorder, Smoldering of indolent multiple myeloma, or hematological malignancies (including leukemia, diffuse large B-cell lymphoma (DLBCL), ABC DLBCL, chronic lymphocytic leukemia (CLL), chronic lymphocytic lymphoma, primary effusion lymphoma, Burkitt lymphoma/leukemia, acute lymphocytic leukemia, B-cell prolymphocytic leukemia, lymphoplasmacytic lymphoma, Waldenstrom's macroglobulinemia (WM), splenic marginal zone lymphoma, multiple myeloma, plasmacytoma, intravascular large B-cell lymphoma).

In some embodiments, the cancer to be treated is selected from glioma, breast cancer, prostate cancer, head and neck squamous cell carcinoma, skin melanomas, ovarian cancer, malignant peripheral nerve sheath tumors (MPNST), and pancreatic cancer. In other embodiments, the cancer to be treated is cancer selected from glioma, breast cancer, prostate cancer, head and neck squamous cell carcinoma, skin melanomas, ovarian cancer, malignant peripheral nerve shealth tumors (MPNST), pancreatic cancer, non-small cell lung cancer (NSCLC) including EGFR-mutant NSCLC, urothelial cancer, liver cancer, bile duct cancer, kidney cancer, colon cancer, esophageal cancer, gastric cancer, gastrointestinal stromal tumors, and hematological malignancies include lymphomas, leukemias, myelomas, myeloproliferative neoplasms and myelodysplastic syndromes. In other embodiments, the cancer is selected from solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, uterine leiomyosarcoma, melanoma etc.), hematological cancers (e.g., lymphoma, leukemia Such as acute lymphoblastic leukemia (ALL), acute myelogenous leukemia (AML) or multiple myeloma), and skin cancer such as cutaneous T-cell lymphoma (CTCL) and cutaneous B-cell lymphoma. Example CTCLs include Sezary syndrome and mycosis fungoides.

Compounds, salts, and compositions described herein are also useful in the treatment of inflammatory or obstructive airways diseases, resulting, for example, in reduction of tissue damage, airways inflammation, bronchial hyperreactivity, remodeling or disease progression. Inflammatory or obstructive airways diseases include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Compounds, salts, and compositions described herein are also useful in the treatment of heteroimmune diseases including, but are not limited to, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis.

Compounds, salts, and compositions described herein are also useful in the treatment of other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. Compounds, salts, and compositions described herein are also useful in the treatment of bronchitis including, but not limited to, acute, arachidic, catarrhal, croupus, chronic or phthinoid bronchitis. Compounds, salts, and compositions described herein are also useful in the treatment of pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Compounds, salts, and compositions described herein are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, Pemphigus vulgaris, Pemphigus foliaceus, paraneoplastic pemphigus, epidermolysis bullosa acquisita, acne vulgaris, and other inflammatory or allergic conditions of the skin.

Compounds, salts, and compositions described herein are also useful in the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acidinduced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, encephalomyelitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments, cardiovascular diseases which can be treated according to the present methods include, but are not limited to, restenosis, cardiomegaly, atherosclerosis, myocardial infarction, ischemic stroke, congestive heart failure, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, and deep venous thrombosis.

In some embodiments, the neurodegenerative disease which can be treated according to the present methods include, but are not limited to, Alzheimer's disease, Parkinson's disease, amyotrophic lateral sclerosis, Huntington's disease, cerebral ischemia, and neurodegenerative disease caused by traumatic injury, glutamate neurotoxicity, hypoxia, epilepsy, treatment of diabetes, metabolic syndrome, obesity, organ transplantation and graft versus host disease.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Preparation of Compounds

The compounds claimed herein were prepared following the procedures outlined in the following schemes. Compound names were generated using the software built into ChemDraw. To the extent that there are discrepancies between the name of a compound and its depicted structure, the depicted chemical structure is to be taken as the appropriate compound.

### Procedures for Syntheses of Cores:

### Synthesis of (3S,6S,10aS)-6-((tert-butoxycarbonyl)amino)-5-oxodecahydropyrrolo[1,2-a]azocine-3-carboxylic acid

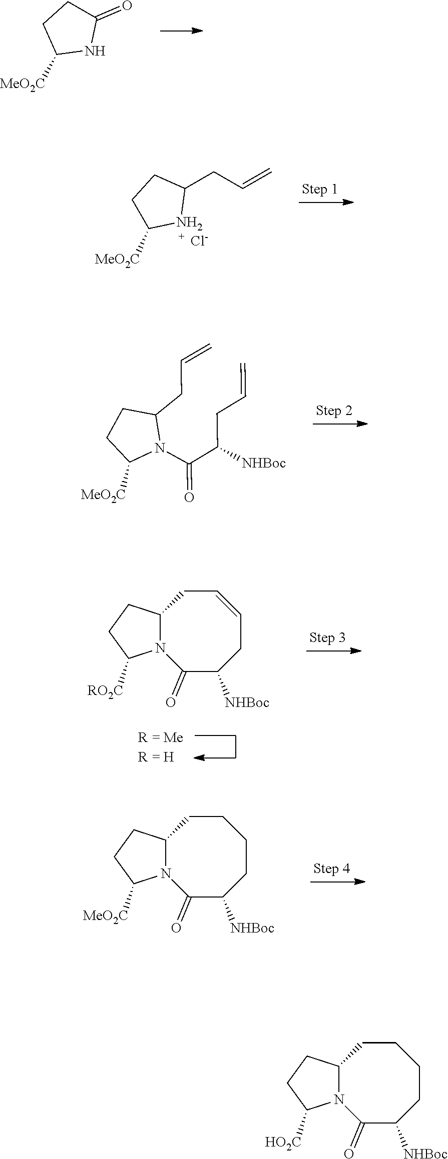

### Step 1: Preparation of Methyl (2S)-5-allyl-1-((S)-2-((tert-butoxycarbonyl)amino)pent-4-enoyl)pyrrolidine-2-carboxylate

To a cooled (0° C.) solution of methyl (2S)-5-allylpyrrolidine-2-carboxylate hydrochloride (200 g, 972 mmol, 1.00 eq) and (S)-2-((tert-butoxycarbonyl)amino)pent-4-enoic acid (209 g, 972 mmol, 1.00 eq) in CH₂Cl₂ (1.60 L) was added Et₃N (406 mL, 2.92 mol, 3.00 eq) and 2-chloro-1-methylpyridinium iodide (CMPI) (273 g, 1.07 mol, 1.10 eq). The solution was warmed to 25° C. and stirred for 1 h. The mixture was poured into water (5.0 L), extracted with CH₂Cl₂ (2.00 L×3). The combined organic layers were washed with brine (2.0 L), dried over Na₂SO₄, filtered and concentrated under reduced pressure. Six individual batches of equal scale were performed in parallel and combined during work up. The resulting residue was purified by column chromatography (petroleum ether:EtOAc=100:1 to 10:1) to give methyl (2S)-5-allyl-1-((S)-2-((tert-butoxycarbonyl)amino)pent-4-enoyl)pyrrolidine-2-carboxylate (1.18 kg, 3.22 mol, 55.2% yield) as yellow oil. ¹H NMR (400 MHz, CDCl₃) δ 5.84-5.78 (m, 2H), 5.17-4.99 (m, 5H), 4.50-4.34 (m, 3H), 3.76-3.70 (m, 3H), 2.50-2.15 (m, 6H), 1.96-1.91 (m, 2H), 1.41 (s, 9H).

The intermediates shown in Table 1 were prepared according the protocol outlined in Step 1 above using (S)-5-oxopyrrolidine-2-carboxylic acid, the appropriate N-protected amino acids [(S)-2-((tert-butoxycarbonyl)amino)but-3-enoic acid) or (S)-2-((tert-butoxycarbonyl)amino)-2-methylpent-4-enoic acid], CMPI, triethylamine, and CH₂Cl₂. The intermediate(s) was purified using standard methods.

TABLE 1

| Name | Structure | LCMS | NMR |
|---|---|---|---|
| methyl (2S)-5-allyl-1-((S)-2- ((tert- butoxycarbonyl)amino)but-3- enoyl)pyrrolidine-2-carboxylate | 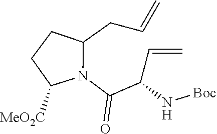 [SMILES: C=CCC1CC[C@@H](C(=O)OC)N1C(=O)[C@H](C=C)NC(=O)OC(C)(C)C] | | ¹H NMR (400 MHz, CDCl₃) δ 5.90-5.75 (m, 2H), 5.46- 5.42 (m, 1H), 5.34-5.26 (m, 2H), 5.14-5.03 (m, 2H), 4.56- 4.48 (m, 1H), 4.31-4.13 (m, 1H), 3.80-3.75 (m, 3H), 2.58- 2.54 (m, 1H), 2.33-1.94 (m, 5H), 1.43 (s, 9H) |
| methyl (2S)-5-allyl-1-((S)-2- ((tert-butoxycarbonyl)amino)- 2-methylpent-4- enoyl)pyrrolidine-2-carboxylate | 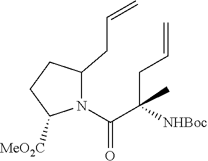 [SMILES: C=CCC1CC[C@@H](C(=O)OC)N1C(=O)[C@](C)(CC=C)NC(=O)OC(C)(C)C] | 281.3 [M + H]⁺ | ¹H NMR (400 MHz, CDCl₃) δ 5.79-5.71 (m, 2H), 5.18- 5.12 (m, 4H), 5.09-4.97 (m, 1H), 4.49 (s, 1H), 4.20 (s, 1H), 3.76 (s, 3H), 2.98 (s, 1H), 2.74- 2.69 (m, 1H), 2.56 (s, 1H), 2.25 (s, 1H), 1.96-1.66 (m, 4H), 1.51 (s, 3H), 1.43 (s, 9H) |

### Step 2: Preparation of Methyl (3S,6S,10aR,Z)-6-((tert-butoxycarbonyl)amino)-5-oxo-1,2,3,5,6,7,10,10a-octahydropyrrolo[1,2-a]azocine-3-carboxylate

To a solution of methyl (2S)-5-allyl-1-((S)-2-((tert-butoxycarbonyl)amino)pent-4-enoyl)pyrrolidine-2-carboxylate (200 g, 546 mmol, 1.00 eq) in CH₂Cl₂ (2.00 L) was added 1^{st} generation Grubbs catalyst (44.9 g, 54.6 mmol, 0.10 eq) at 25° C. The solution was subsequently heated to 50° C. and stirred for 36 h. Six individual batches of equal scale were performed in parallel and combined during work up. The combined reaction mixtures were concentrated to give a residue. The residue was purified by column chromatography (petroleum ether:EtOAc=100:1 to 0:1) twice to give a crude product. The crude product was triturated with petroleum ether (2.00 L) for 12 h and filtered. The filter cake was dried under reduced pressure to give methyl (3S,6S,10aR,Z)-6-((tert-butoxycarbonyl)amino)-5-oxo-1,2,3,5,6,7,10,10a-octahydropyrrolo[1,2-a]azocine-3-carboxylate (510 g, 1.40 mol, 51.3% yield, 92.9% purity) as a solid. LCMS (ESI) m/z=361.2 [M+Na]⁺; ¹H NMR (400 MHz, CDCl₃) δ 5.82-5.80 (m, 1H), 5.73-5.71 (m, 1H), 5.58-5.56 (m, 1H), 4.88-4.85 (m, 1H), 4.52-4.49 (m, 1H), 4.16-4.14 (m, 1H), 3.71 (s, 3H), 2.81-2.74 (m, 2H), 2.45-2.38 (m, 1H), 2.33-2.24 (m, 1H), 2.14-2.05 (m, 2H), 1.98-1.93 (m, 2H), 1.43 (s, 9H).

The intermediates shown in Table 2 were prepared according to the representative protocol described above in Step 2 with appropriate modifications.

TABLE 2

| Name | Structure | LCMS | NMR |
|---|---|---|---|
| methyl (3S,6S,9aR)-6- ((tert- butoxycarbonyl)amino)- 5-oxo-2,3,5,6,9,9a- hexahydro-1H- pyrrolo[1,2-a]azepine-3- carboxylate | 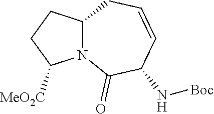 [SMILES: COC(=O)[C@@H]1CC[C@@H]2CC=C[C@H](NC(=O)OC(C)(C)C)C(=O)N21] | 225.1 [(M − 100) + H]⁺ | ¹H NMR (400 MHz, CDCl₃) δ 5.75 (d, J = 6.4 Hz, 1H), 5.65-5.61 (m, 1H), 5.44-5.35 (m, 2H), 4.58-4.54 (m, 1H), 4.46-4.41 (m, 1H), 3.75 (s, 3H), 2.51-2.42 (m, 1H), 2.33-2.22 (m, 3H), 2.10-2.06 (m, 1H), 1.82- 1.77 (m, 1H), 1.45 (s, 9H). |
| methyl (3S,6S,10aR,Z)- 6-((tert- butoxycarbonyl)amino)- 6-methyl-5-oxo- 1,2,3,5,6,7,10,10a- octahydropyrrolo[1,2- a]azocine-3-carboxylate | 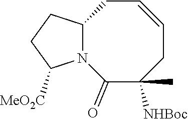 [SMILES: COC(=O)[C@@H]1CC[C@@H]2C/C=C\C[C@](C)(NC(=O)OC(C)(C)C)C(=O)N21] | 353.3 [M + H]⁺ | ¹H NMR (400 MHz, CDCl₃) δ 6.34 (s, 1H), 5.78-5.68 (m, 2H), 4.81- 4.75 (m, 1H), 4.38 (t, J = 8.0 Hz, 1H), 3.73 (s, 3H), 3.46-3.41 (m, 1H), 2.63-2.50 (m, 2H), 2.33- 2.12 (m, 3H), 2.97- 2.82 (m, 2H), 1.59 (s, 3H), 1.41 (s, 9H) |

### Step 3: Preparation of Methyl (3S,6S,10aS)-6-((tert-butoxycarbonyl)amino)-5-oxodecahydropyrrolo[1,2-a]azocine-3-carboxylate

To a solution of methyl (3S,6S,10aR,Z)-6-((tert-butoxycarbonyl)amino)-5-oxo-1,2,3,5,6,7,10,10a-octahydropyrrolo[1,2-a]azocine-3-carboxylate (47.9 g, 137 mmol, 96.8% purity, 1.00 eq) in EtOAc (500 mL) was added 10% Pd/C (9.58 g) under N₂ (g). The suspension was degassed under vacuum and purged with H₂ (g) several times. The mixture was stirred at 25° C. under H₂ (g) (50 psi) for 16 h. The mixture was filtered, and the filtrate was concentrated to give methyl (3S,6S,10aS)-6-((tert-butoxycarbonyl)amino)-5-oxodecahydropyrrolo[1,2-a]azocine-3-carboxylate (48.0 g) as brown oil. LCMS (ESI) m/z=341.0 [M+H]⁺; ¹H NMR (400 MHz, CDCl₃) δ 5.43 (d, J=8.4 Hz, 1H), 4.69-4.57 (m, 1H), 4.50-4.41 (m, 1H), 4.27-4.18 (m, 1H), 3.76 (s, 3H), 2.34-2.25 (m, 1H), 2.21-2.11 (m, 1H), 2.04-1.68 (m, 8H), 1.64-1.57 (m, 2H), 1.43 (s, 9H).

The intermediates shown in Table 3 were prepared according to the protocol described above in Step 3 with appropriate modifications.

TABLE 3

| Name | Structure | LCMS | NMR |
|---|---|---|---|
| methyl (3S,6S,9aS)-6-((tert- butoxycarbonyl)amino)-5- oxooctahydro-1H- pyrrolo[1,2-a]azepine-3- carboxylate | 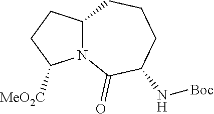 [SMILES: COC(=O)[C@@H]1CC[C@@H]2CCC[C@H](NC(=O)OC(C)(C)C)C(=O)N21] | 327.2 [M + H]⁺ | ¹H NMR (400 MHz, CDCl₃) δ 4.58 (dd, J = 8.4, 3.6 Hz, 1H), 4.22 (br d, J = 11.2 Hz, 1H), 3.97 (br d, J = 10.0 Hz, 1H), 3.72 (s, 3H), 2.36-2.23 (m, 1H), 2.11 (br dd, J = 8.8, 1.2 Hz, 1H), 2.07-1.96 (m, 2H), 1.89-1.56 (m, 6H), 1.44 (s, 9H) |
| methyl (3S,6S,10aS)-6- ((tert- butoxycarbonyl)amino)-6- methyl-5- oxodecahydropyrrolo[1,2- a]azocine-3-carboxylate | 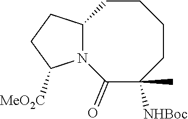 [SMILES: COC(=O)[C@@H]1CC[C@@H]2CCCC[C@](C)(NC(=O)OC(C)(C)C)C(=O)N21] | 355.2 [M + H]⁺ | ¹H NMR (400 MHz, CDCl₃) δ 6.22 (s, 1H), 4.62-4.59 (m, 1H), 4.33 (t, J = 9.2 Hz, 1H), 3.76 (s, 3H), 2.95 (d, J = 16.4 Hz, 1H), 2.24-2.02 (m, 3H), 1.94-1.77 (m, 4H), 1.72- 1.66 (m, 2H), 1.64-1.57 (m, 1H), 1.55 (s, 3H), 1.52-1.44 (m, 1H), 1.41 (s, 9H) |

### Step 4: Preparation of (3S,6S,10aS)-6-((tert-butoxycarbonyl)amino)-5-oxodecahydropyrrolo[1,2-a]azocine-3-carboxylic acid

To a cooled (0° C.) solution of methyl (3S,6S,10aS)-6-((tert-butoxycarbonyl)amino)-5-oxodecahydropyrrolo[1,2-a]azocine-3-carboxylate (17.0 g, 49.9 mmol, 1.00 eq) in 1,4-dioxane (85.0 mL) was added a 2 M aqueous solution of LiOH—H₂O (74.9 mL, 3.00 eq). The mixture was allowed to gradually warm to room temperature stirred at 25° C. for 16 h. The reaction mixture was acidified with 1.0 N HCl to pH ˜4.0. The mixture was extracted with EtOAc (3×). The combined organic layers were dried over Na₂SO₄ and concentrated to give a residue. The residue was dissolved with water (150 mL) and acetonitrile (30 mL). The mixture was lyophilized to give (3S,6S,10aS)-6-((tert-butoxycarbonyl)amino)-5-oxodecahydropyrrolo[1,2-a]azocine-3-carboxylic acid (15.5 g, 44.8 mmol, 92.3% yield for two steps, 94.3% purity) as a grey solid. LCMS (ESI) m/z=327.0 [M+H]⁺; ¹H NMR (400 MHz, CDCl₃) δ 7.01 (s, 1H), 5.53 (d, J=8.0 Hz, 1H), 4.72-4.62 (m, 1H), 4.54 (t, J=8.6 Hz, 1H), 4.32-4.21 (m, 1H), 2.32-2.12 (m, 3H), 2.05-1.95 (m, 1H), 1.85-1.55 (m, 8H), 1.43 (s, 9H).

### Step 5: Preparation of (3S,6S,10aR,Z)-6-((tert-butoxycarbonyl)amino)-5-oxo-1,2,3,5,6,7,10,10a-octahydropyrrolo[1,2-a]azocine-3-carboxylic acid

(3S,6S,10aR,Z)-6-((tert-butoxycarbonyl)amino)-5-oxo-1,2,3,5,6,7,10,10a-octahydropyrrolo[1,2-a]azocine-3-carboxylic acid was prepared using the hydrolysis conditions described above in step 4 for the preparation of (3S,6S,10aS)-6-((tert-butoxycarbonyl)amino)-5-oxodecahydropyrrolo[1,2-a]azocine-3-carboxylic acid and starting from methyl (3S,6S,10aR,Z)-6-((tert-butoxycarbonyl)amino)-5-oxo-1,2,3,5,6,7,10,10a-octahydropyrrolo[1,2-a]azocine-3-carboxylate. Starting from (3S,6S,10aR,Z)-6-((tert-butoxycarbonyl)amino)-5-oxo-1,2,3,5,6,7,10,10a-octahydropyrrolo[1,2-a]azocine-3-carboxylic acid (35.0 g, 103 mmol, 1.00 equiv), LiOH·H₂O (8.64 g, 3.00 eq), and THF (150 mL)/water (50 mL) afforded (3S,6S,10aR,Z)-6-((tert-butoxycarbonyl)amino)-5-oxo-1,2,3,5,6,7,10,10a-octahydropyrrolo[1,2-a]azocine-3-carboxylic acid (31.1 g, 99.1 mmol, 96.1% yield) as white solids. LCMS (ESI) m/z=325.2 [M+H]⁺;

The intermediate(s) shown in Table 4 were prepared using the hydrolysis conditions described above in Step 4 for the preparation of (3S,6S,10aS)-6-((tert-butoxycarbonyl)amino)-5-oxodecahydropyrrolo[1,2-a]azocine-3-carboxylic acid and starting from the appropriate starting materials and conditions.

TABLE 4

| Name | Structure | LCMS | NMR |
|---|---|---|---|
| (3S,6S,9aS)-6-((tert- butoxycarbonyl)amino)-5- oxooctahydro-1H- pyrrolo[1,2-a]azepine-3- carboxylic acid | 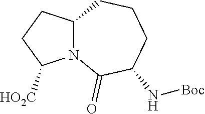 [SMILES: CC(C)(C)OC(=O)N[C@H]1CCC[C@H]2CC[C@@H](C(=O)O)N2C1=O] | 313.2 [M + H]⁺ | ¹H NMR (400 MHz, DMSO- d₆) δ 12.33-12.61 (m, 1H) 6.59 (d, J = 8.0 Hz, 1H) 4.36 (m, 1H) 4.01-4.15 (m, 1H) 3.88 (m, 1H) 2.11-2.26 (m, 1H) 1.96-2.07 (m, 1H) 1.84- 1.92 (m, 2H) 1.71 (m, 2H) 1.66 (m, 2H) 1.41-1.55 (m, 2H) 1.37 (s, 9H) |
| (3S,6S,9aR)-6-((tert- butoxycarbonyl)amino)-5- oxo-2,3,5,6,9,9a-hexahydro- 1H-pyrrolo[1,2-a]azepine-3- carboxylic acid | 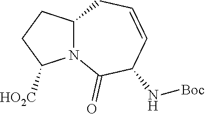 [SMILES: CC(C)(C)OC(=O)N[C@H]1C=CC[C@H]2CC[C@@H](C(=O)O)N2C1=O] | 333.2 [M + Na]⁺ | ¹H NMR (400 MHz, methanol-d₄) δ 6.34-6.21 (m, 1H), 5.77-5.58 (m, 1H), 5.42-5.27 (m, 1H), 4.68- 4.43 (m, 2H), 4.13-3.98 (m, 1H), 2.69-2.50 (m, 1H), 2.35-2.21 (m, 3H), 2.13- 2.03 (m, 2H), 1.44 (s, 9H) |
| (3S,6S,10aS)-6-((tert- butoxycarbonyl)amino)-6- methyl-5- oxodecahydropyrrolo[1,2- a]azocine-3-carboxylic acid | 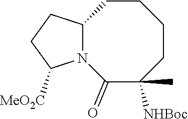 | 341.1 [M + H]⁺ | ¹H NMR (400 MHz, CDCl₃) δ 6.15 (s, 1H), 4.65-4.60 (m, 1H), 4.48 (t, J = 7.6 Hz, 1H), 2.91 (d, J = 16.0 Hz, 1H), 2.26-2.16 (m, 3H), 2.11- 2.04 (m, 1H), 1.89-1.75 (m, 2H), 1.72-1.61 (m, 4H), 1.59 (m, 3H), 1.54-1.48 (m, 1H), 1.43 (s, 9H) |

### Synthesis of methyl (3S,6S,10aR,Z)-6-((tert-butoxycarbonyl)amino)-5-oxo-1,2,3,5,6,7,8,10a-octahydropyrrolo[1,2-a]azocine-3-carboxylate

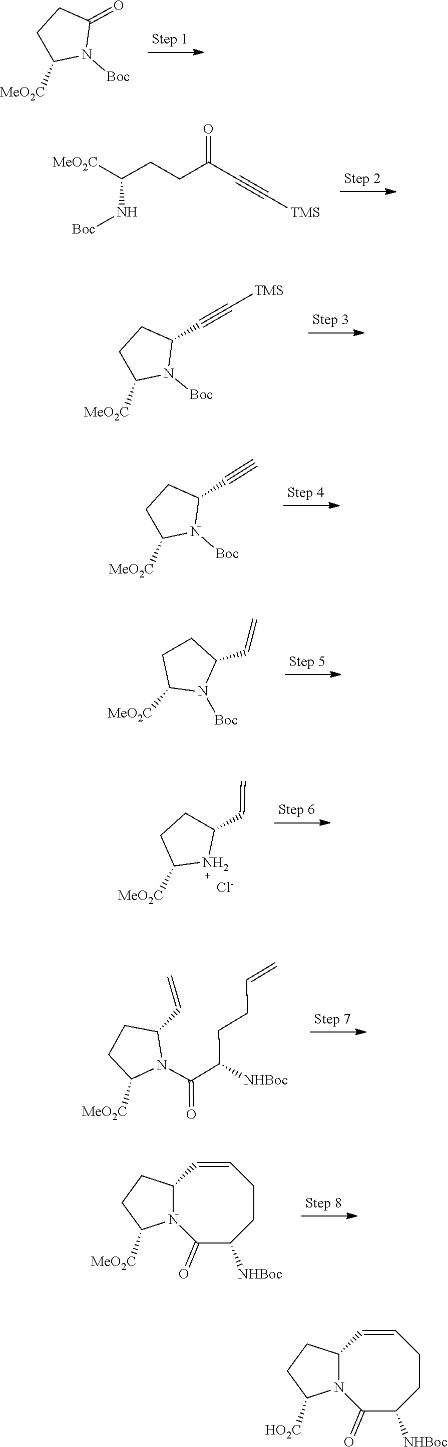

### Step 1: Preparation of Methyl (S)-2-((tert-butoxycarbonyl)amino)-5-oxo-7-(trimethylsilyl)hept-6-ynoate

To a cooled (0° C.) solution of isopropylmagnesium chloride (2.0 M, 9.04 L, 1.10 eq) and THF (4.00 L) was added ethynyltrimethylsilane (1.86 kg, 18.9 mol, 2.62 L, 1.15 eq). The reaction mixture was stirred for 1 h, followed by slow addition of a solution of 1-(tert-butyl) 2-methyl (S)-5-oxopyrrolidine-1,2-dicarboxylate (4.00 kg, 16.4 mol, 1.00 eq) in THF (8.0 L) over 1.5 h. After stirring for an additional 30 min, the reaction mixture was transferred into a stirred and cooled (0° C.) biphasic mixture of iPAc (5.00 L) and 20% aqueous NH₄Cl (16.0 L). The biphasic mixture was separated, and the aqueous layer was extracted with iPAc (8.00 L). The combined organic layers were washed with 20% aqueous NH₄Cl solution (8.00 L), brine (8.00 L), dried over Na₂SO₄, filtered and concentrated under reduced pressure to give methyl (S)-2-((tert-butoxycarbonyl)amino)-5-oxo-7-(trimethylsilyl)hept-6-ynoate (5.50 kg) as yellow oil. ¹H NMR (400 MHz, CDCl₃) δ 5.11-5.09 (m, 1H), 4.32-4.30 (m, 1H), 3.77-3.72 (m, 3H), 2.72-2.64 (m, 2H), 2.04-2.01 (m, 1H), 1.73-1.71 (m, 1H), 1.41 (s, 9H), 0.24 (s, 9H).

### Step 2: Preparation of 1-(tert-butyl) 2-methyl (2S,5R)-5-((trimethylsilyl)ethynyl)pyrrolidine-1,2-dicarboxylate

To a suspension of NaBH(OAc)₃ (4.03 kg, 19.0 mol, 1.30 eq) in iPAc (5.00 L) was added a solution of methyl (S)-2-((tert-butoxycarbonyl)amino)-5-oxo-7-(trimethylsilyl)hept-6-ynoate in iPAc (20.0 L). The batch was cooled to (−10° C.), followed by slow addition of TFA (7.18 kg, 62.9 mol, 4.66 L, 4.30 eq) over 1.5 h. The mixture was warmed to 10° C. and stirred for an additional 2 h. The reaction mixture was transferred to a cooled (0° C.) solution of 25% aqueous K₂HPO₄ (40.0 L). The pH of the suspension was adjusted to pH 6-7 with aqueous saturated NaHCO₃. The biphasic mixture was separated, and the aqueous layer was extracted with iPAc (8.00 L). The combined organic layers were washed with 25% K₂HPO₄ solution (8.00 L), brine (5.00 L), dried over Na₂SO₄, filtered, and concentrated under reduced pressure to give a residue. The residue was purified by silica gel column chromatography (petroleum ether/EtOAc=30/1 to 3/1) to yield 1-(tert-butyl) 2-methyl (2S,5R)-5-((trimethylsilyl)ethynyl)pyrrolidine-1,2-dicarboxylate (2.27 kg, 6.97 mol, 42.4% yield) as yellow oil. ¹H NMR (400 MHz, DMSO-d₆) δ 4.49-4.32 (m, 1H), 4.16 (s, 1H), 3.63 (s, 3H), 2.16 (s, 2H), 1.95-1.88 (m, 2H), 1.40-1.32 (m, 9H), 0.13 (s, 9H).

### Step 3: Preparation of 1-(tert-butyl) 2-methyl (2S,5R)-5-ethynylpyrrolidine-1,2-dicarboxylate

To a cooled (0° C.) solution of 1-(tert-butyl) 2-methyl (2S,5R)-5-((trimethylsilyl)ethynyl)pyrrolidine-1,2-dicarboxylate (500 g, 1.54 mol, 1.00 eq) in THF (2.00 L) was added a 1.0 M solution of TBAF (1.84 L, 1.20 eq) in THF and the mixture was subsequently stirred for 1 h. Four batches of equal scale were performed in parallel, and the reaction mixtures were combined and concentrated under reduced pressure to give a residue. The residue was purified by silica gel column chromatography (petroleum ether/EtOAc=1/0 to 0/1) to yield 1-(tert-butyl) 2-methyl (2S,5R)-5-ethynylpyrrolidine-1,2-dicarboxylate (I-14) (920 g, 2.95 mol, 48.0% yield, 81.2% purity) as yellow oil. ¹H NMR (400 MHz, CDCl₃) δ 4.62-4.50 (m, 1H), 4.30-4.20 (m, 1H), 3.74-3.70 (m, 3H), 2.31-2.14 (m, 5H), 1.47-1.40 (m, 9H).

### Step 4: Preparation of 1-(tert-butyl) 2-methyl (2S,5R)-5-vinylpyrrolidine-1,2-dicarboxylate

To a suspension of 1-(tert-butyl) 2-methyl (2S,5R)-5-ethynylpyrrolidine-1,2-dicarboxylate (150 g, 592 mmol, 1.00 eq) in EtOAc (1.50 L) under N₂ (g) was added Lindlar's catalyst (7.50 g, 1.82 mmol, 5.0% wt) and quinoline (163 g, 1.27 mol, 150 mL, 2.14 eq). The suspension was degassed under vacuum and purged with H₂ (g) (3×). The mixture was stirred under H₂ (g) (50 psi) for 1 h. Six batches of equal scale were performed in parallel, filtered, and the filtrates were combined during workup. The mixture was filtered, the filtrate was washed with aqueous 1N HCl (9.00 L), separated, and concentrated. The combined residues were purified by silica gel column chromatography (petroleum ether/EtOAc=I/O to 0/1) to yield 1-(tert-butyl) 2-methyl (2S,5R)-5-vinylpyrrolidine-1,2-dicarboxylate (745 g, 2.23 mol, 62.8% yield, 76.5% purity) as yellow oil. ¹H NMR (400 MHz, DMSO-d₆) δ 5.84-5.76 (m, 1H), 5.34-5.27 (m, 1H), 5.06-5.04 (m, 1H), 4.29-4.17 (m, 2H), 3.65-3.62 (m, 3H), 2.16-2.14 (m, 2H), 1.79-1.78 (m, 1H), 1.69-1.65 (m, 1H), 1.34-1.32 (m, 9H).

### Step 5: Preparation of Methyl (2S,5R)-5-vinylpyrrolidine-2-carboxylate

To a solution of 1-(tert-butyl) 2-methyl (2S,5R)-5-vinylpyrrolidine-1,2-dicarboxylate (245 g, 959 mmol, 1.00 eq) in EtOAc (1.25 L) was added a solution of 4.0 M solution of HCl in EtOAc (959 mL, 4.00 eq). Three batches of equal scale were performed in parallel, and the mixture was stirred at 25° C. for 2 h. The reaction mixtures were combined and concentrated under reduced pressure to give product methyl (2S,5R)-5-vinylpyrrolidine-2-carboxylate (520 g, HCl) was obtained as an off-white solid. ¹H NMR (400 MHz, DMSO-d₆) δ 6.03-5.95 (m, 1H), 5.44-5.33 (m, 2H), 4.49-4.45 (m, 1H), 4.11-4.05 (m, 1H), 3.76 (s, 3H), 2.31-2.28 (m, 1H), 2.16-2.10 (m, 1H), 1.81-1.76 (m, 1H).

### Step 6: Preparation of Methyl (2S,5R)-1-((S)-2-((tert-butoxycarbonyl)amino)hex-5-enoyl)-5-vinylpyrrolidine-2-carboxylate

To a solution of (S)-2-((tert-butoxycarbonyl)amino)hex-5-enoic acid (80.6 g, 352 mmol, 1.00 eq) in CH₂Cl₂ (674 mL) was added methyl (2S,5R)-5-vinylpyrrolidine-2-carboxylate (67.4 g, 352 mmol, 1.00 eq, HCl) and Et₃N (147 mL, 1.06 mol, 3.00 eq). The mixture was cooled to 0° C. and CMPI (98.8 g, 387 mmol, 1.10 eq) was added. The reaction mixture was subsequently warmed to 25° C. and stirred for 3 h. The reaction mixture was poured into water (500 mL), extracted with CH₂Cl₂ (200 mL×3). The combined organic layers were washed with saturated aqueous NH₄Cl (300 mL×2), dried over Na₂SO₄, filtered and concentrated under reduced pressure to give a residue. The residue was purified by column chromatography (petroleum ether/EtOAc=50/1 to 5/1) to give methyl (2S,5R)-1-((S)-2-((tert-butoxycarbonyl)amino)hex-5-enoyl)-5-vinylpyrrolidine-2-carboxylate (115 g, 247 mmol, 70.2% yield, 78.9% purity) as yellow oil. LCMS (ESI) m/z=367.2 [M+H]⁺; ¹H NMR (400 MHz, CDCl₃) δ 6.03-5.94 (m, 1H), 5.53 (d, J=17.2 Hz, 1H), 5.23 (d, J=10.4 Hz, 1H), 5.03-4.94 (m, 3H), 4.81 (t, J=12.0 Hz, 1H), 4.53-4.40 (m, 2H), 3.75 (s, 3H), 2.25-2.18 (m, 2H), 2.16-2.12 (m, 1H), 2.10-2.07 (m, 1H), 2.05 (s, 1H), 2.00-1.97 (m, 1H), 1.89-1.84 (m, 1H), 1.80-1.73 (m, 1H), 1.43 (s, 9H).

The intermediates shown in Table 5 were synthesized using methyl (2S,5R)-5-vinylpyrrolidine-2-carboxylate, appropriate N-protected amino acids, CMPI, triethylamine, and CH₂Cl₂ under the reaction conditions described above. The intermediate(s) was purified using standard methods.

TABLE 5

| Name | Structure | LCMS |
|---|---|---|
| (2S,5R)-1-((S)-2-((tert-butoxycarbonyl)amino)- 4-methylpent-4-enoyl)-5-vinylpyrrolidine-2- carboxylic acid | 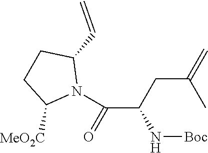 [SMILES: C=C[C@H]1CC[C@@H](C(=O)OC)N1C(=O)[C@H](CC(=C)C)NC(=O)OC(C)(C)C] | 367.2 [M + H]⁺ |
| methyl (2S,5R)-1-((S)-2-((tert- butoxycarbonyl)amino)pent-4-enoyl)-5- vinylpyrrolidine-2-carboxylate | 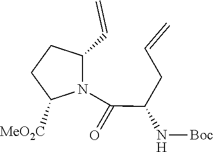 [SMILES: C=CC[C@H](NC(=O)OC(C)(C)C)C(=O)N1[C@@H](C=C)CC[C@H]1C(=O)OC] | 353.0 [M + H]⁺ |
| methyl (2S,5R)-1-((S)-2-((tert- butoxycarbonyl)amino)but-3-enoyl)-5- vinylpyrrolidine-2-carboxylate | 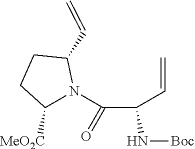 [SMILES: C=C[C@H](NC(=O)OC(C)(C)C)C(=O)N1[C@@H](C=C)CC[C@H]1C(=O)OC] | 239.0 [(M − 100) + H]⁺ |

### Step 7: Preparation of Methyl (3S,6S,10aR,Z)-6-((tert-butoxycarbonyl)amino)-5-oxo-1,2,3,5,6,7,8,10a-octahydropyrrolo[1,2-a]azocine-3-carboxylate

To a solution of methyl (2S,5R)-1-((S)-2-((tert-butoxycarbonyl)amino)hex-5-enoyl)-5-vinylpyrrolidine-2-carboxylate (38.2 g, 104 mmol, 1.00 eq) in CH₂Cl₂ (1.90 L) was added 1st generation Grubb's catalyst (8.58 g, 10.4 mmol, 0.10 eq). The mixture was heated to 50° C. and stirred for 12 h. Three batches of equal scale were performed in parallel. The reaction mixtures were subsequently combined and concentrated to give a residue. The residue was purified by column chromatography (petroleum ether/EtOAc=100/1 to 7/3) to give the crude product as a gray solid. The crude product (62.0 g) was triturated with petroleum ether (122 mL) for 1.5 h. The mixture was filtered and the filter cake was dried under vacuum to give methyl (3S,6S,10aR,Z)-6-((tert-butoxycarbonyl)amino)-5-oxo-1,2,3,5,6,7,8,10a-octahydropyrrolo[1,2-a]azocine-3-carboxylate (50.0 g, 50.3% yield) as a gray solid. LCMS (ESI) m/z=339.3 [M+H]⁺; ¹H NMR (400 MHz, CDCl₃) δ 5.78-5.71 (m, 1H), 5.48 (d, J=12.0 Hz, 1H), 5.28 (d, J=8.8 Hz, 1H), 4.75-4.68 (m, 2H), 4.62 (t, J=6.4 Hz, 1H), 3.74 (s, 3H), 3.10-3.07 (m, 1H), 2.30-2.24 (m, 1H), 2.15-2.07 (m, 2H), 2.00-1.88 (m, 2H), 1.64-1.57 (m, 1H), 1.42 (s, 9H).

The intermediates shown in Table 6 were synthesized using the appropriate starting materials and reagents under conditions described above for the preparation of methyl (3S,6S,10aR,Z)-6-((tert-butoxycarbonyl)amino)-5-oxo-1,2,3,5,6,7,10,10a-octahydropyrrolo[1,2-a]azocine-3-carboxylate.

TABLE 6

| Name | Structure | LCMS |
|---|---|---|
| methyl (3S,6S,9aR)-6-((tert- butoxycarbonyl)amino)-8-methyl-5-oxo- 2,3,5,6,7,9a-hexahydro-1H-pyrrolo[1,2- a]azepine-3-carboxylate | 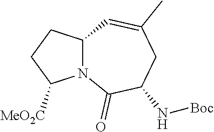 [SMILES: COC(=O)[C@@H]1CC[C@@H]2C=C(C)C[C@H](NC(=O)OC(C)(C)C)C(=O)N21] | 361.1 [M + Na]⁺ |
| methyl (3S,6S,9aR)-6-((tert- butoxycarbonyl)amino)-5-oxo-2,3,5,6,7,9a- hexahydro-1H-pyrrolo[1,2-a]azepine-3- carboxylate | 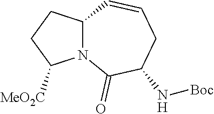 [SMILES: COC(=O)[C@@H]1CC[C@@H]2C=CC[C@H](NC(=O)OC(C)(C)C)C(=O)N21] | 347.1 [M + Na]⁺ |
| methyl (3S,6S,8aR)-6-((tert- butoxycarbonyl)amino)-5-oxo-1,2,3,5,6,8a- hexahydroindolizine-3-carboxylate | 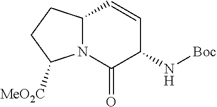 [SMILES: COC(=O)[C@@H]1CC[C@@H]2C=C[C@H](NC(=O)OC(C)(C)C)C(=O)N21] | 211.2 [(M − 100) + H]⁺ |

### Step 8: Preparation of (3S,6S,10aR,Z)-6-((tert-butoxycarbonyl)amino)-5-oxo-1,2,3,5,6,7,8,10a-octahydropyrrolo[1,2-a]azocine-3-carboxylic acid

(3S,6S,10aR,Z)-6-((tert-butoxycarbonyl)amino)-5-oxo-1,2,3,5,6,7,8,10a-octahydropyrrolo[1,2-a]azocine-3-carboxylic acid was prepared using the hydrolysis conditions described previously in Step 4 for the preparation (3S,6S,10aS)-6-((tert-butoxycarbonyl)amino)-5-oxodecahydropyrrolo[1,2-a]azocine-3-carboxylic acid. Using methyl (3S,6S,10aR,Z)-6-((tert-butoxycarbonyl)amino)-5-oxo-1,2,3,5,6,7,8,10a-octahydropyrrolo[1,2-a]azocine-3-carboxylate, LiOH (185 mg, 4.41 mmol, 3 eq), and THF/water (9 mL/3 mL) afforded (3S,6S,10aR,Z)-6-((tert-butoxycarbonyl)amino)-5-oxo-1,2,3,5,6,7,8,10a-octahydropyrrolo[1,2-a]azocine-3-carboxylic acid (488 mg, 1.50 mmol) as white solids. LCMS (ESI) m/z=339.1 [M+H]⁺.

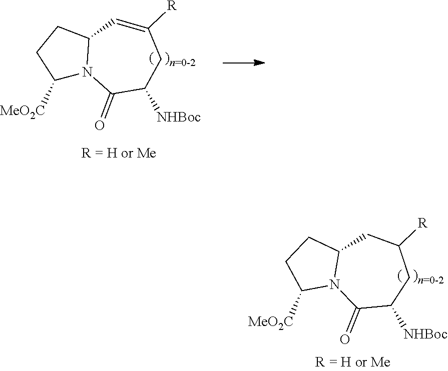

The intermediates shown in Table 7 were synthesized using the appropriate starting materials and reagents under the hydrogenation conditions described in Step 3 for the preparation methyl (3S,6S,10aS)-6-((tert-butoxycarbonyl)amino)-5-oxodecahydropyrrolo[1,2-a]azocine-3-carboxylate.

TABLE 7

| Name | Structure | LCMS |
|---|---|---|
| methyl (3S,6S,9aR)-6-((tert- butoxycarbonyl)amino)-8-methyl-5- oxooctahydro-1H-pyrrolo[1,2-a]azepine-3- carboxylate | 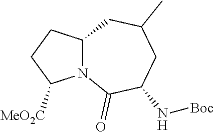 | 241.1 [(M − 100) + H]⁺ |

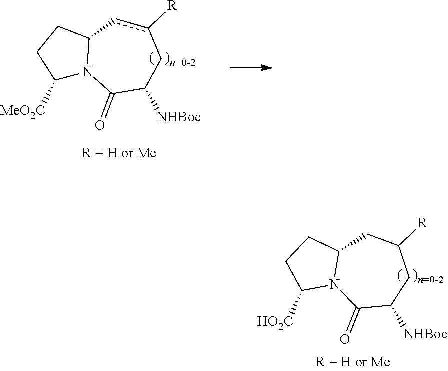

The intermediates shown in Table 8 were synthesized using the appropriate starting materials and reagents under the hydrolysis conditions described in Step 4 for the preparation (3S,6S,10aS)-6-((tert-butoxycarbonyl)amino)-5-oxodecahydropyrrolo[1,2-a]azocine-3-carboxylic acid.

TABLE 8

| Name | Structure |
|---|---|
| (3S,6S,9aR)-6-((tert- butoxycarbonyl)amino)- 8-methyl-5-oxooctahydro- 1H-pyrrolo[1,2- a]azepine-3-carboxylic acid | 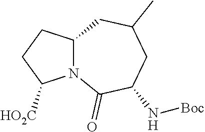 |
| (3S,6S,8aS)-6-((tert- butoxycarbonyl)amino)- 5-oxooctahydro- indolizine-3-carboxylic acid | 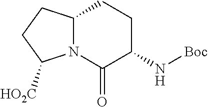 [SMILES: CC(C)(C)OC(=O)N[C@H]1CC[C@H]2CC[C@@H](C(=O)O)N2C1=O] |
| (3S,6S,9aR)-6-((tert- butoxycarbonyl)amino)- 5-oxo-2,3,5,6,7,9a-hexahydro-1H- pyrrolo[1,2-a]azepine- 3-carboxylic acid | 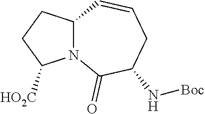 [SMILES: CC(C)(C)OC(=O)N[C@H]1CC=C[C@H]2CC[C@@H](C(=O)O)N2C1=O] |

### Synthesis of (4S,7S,11aS)-7-((tert-butoxycarbonyl)amino)-6-oxodecahydro-2H-pyrido[1,2-a]azocine-4-carboxylic acid

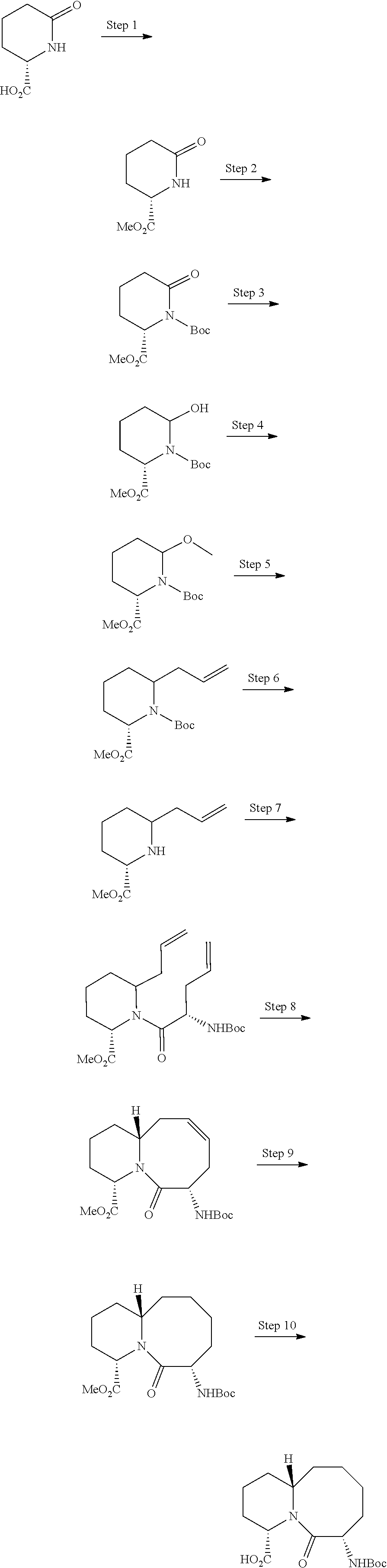

### Step 1: Preparation of Methyl (S)-6-oxopiperidine-2-carboxylate

To a cooled (0° C.) solution of MeOH (1.50 L) was sequentially added SOCl₂ (137 g, 1.15 mol, 83.6 mL, 1.10 eq) and (S)-6-oxopiperidine-2-carboxylic acid (150 g, 1.05 mol, 1.00 eq). The mixture was warmed to 25° C. and stirred for 12 h. Two batches of equal scale were performed in parallel. The reaction mixtures were combined and concentrated in vacuo. The resulting residue was dissolved in a mixture of toluene (1.15 L) and Et₃N (292 mL, 2.10 mol, 2.00 eq). The mixture was stirred at 25° C. for 30 mins, then filtered and the filtrate was concentrated to give methyl (S)-6-oxopiperidine-2-carboxylate (282 g) as yellow oil. LCMS (ESI) m/z=158.2 [M+H]⁺; ¹H NMR (400 MHz, CDCl₃) δ 6.53 (s, 1H), 4.10 (t, J=5.60 Hz, 1H), 3.77 (s, 3H), 2.43-2.33 (m, 2H), 2.20-2.15 (m, 1H), 1.89-1.77 (m, 3H).

### Step 2: Preparation of 1-(tert-butyl) 2-methyl (S)-6-oxopiperidine-1,2-dicarboxylate

To a solution of methyl (S)-6-oxopiperidine-2-carboxylate (141 g, 897 mmol, 1.00 eq) in acetonitrile (1.41 L) was added Boc₂O (235 g, 1.08 mol, 247 mL, 1.20 eq), DMAP (21.9 g, 179 mmol, 0.20 eq) and Et₃N (136 g, 1.35 mol, 187 mL, 1.50 eq) at 25° C. The mixture was stirred at 25° C. for 3 h. Two batches of equal scale were performed in parallel, the reaction mixtures were combined, and concentrated under reduced pressure to give a residue. The residue was purified by column chromatography (petroleum ether/EtOAc=100/1 to 80/1) to give 1-(tert-butyl) 2-methyl (S)-6-oxopiperidine-1,2-dicarboxylate (431 g, 1.67 mol, 79.5% yield over two steps, 99.4% purity) as a yellow solid. LCMS (ESI) m/z=158.2 [(M-Boc)+H]⁺; ¹H NMR: (400 MHz, CDCl₃) δ 4.71-4.69 (m, 1H), 3.76 (s, 3H), 2.60-2.43 (m, 2H), 2.18-2.14 (m, 1H), 2.09-2.00 (m, 1H), 1.81-1.72 (m, 2H), 1.49 (s, 9H).

### Step 3: Preparation of 1-(tert-butyl) 2-methyl (2S)-6-hydroxypiperidine-1,2-dicarboxylate

To a cooled (−78° C.) solution of 1-(tert-butyl) 2-methyl (S)-6-oxopiperidine-1,2-dicarboxylate (120 g, 464 mmol, 99.4% purity, 1.00 eq) in THF (960 mL) was added LiEt₃BH (1 M, 510 mL, 1.10 eq). The mixture was stirred at for 1 h under N₂ (g). The reaction mixture was poured into saturated aqueous NH₄Cl solution (4.50 L) and extracted with EtOAc (2.00 L×2). Three batches of equal scale were performed and worked-up in a similar manner. The combined organic layers were washed with brine (1.50 L×2), dried over Na₂SO₄, filtered, and concentrated under reduced pressure to give 1-(tert-butyl) 2-methyl (2S)-6-hydroxypiperidine-1,2-dicarboxylate (487 g) as yellow oil. ¹H NMR (400 MHz, DMSO-d₆) δ 5.55 (s, 1H), 5.34 (s, 1H), 4.58 (s, 1H), 3.58 (m, 3H), 2.11 (d, J=13.2 Hz, 1H), 1.83-1.73 (m, 1H), 1.65 (d, J=1.2 Hz, 2H), 1.57-1.51 (m, 2H), 1.41 (s, 9H).

### Step 4: Preparation of 1-(tert-butyl) 2-methyl (2S)-6-methoxypiperidine-1,2-dicarboxylate

To a solution of 1-(tert-butyl) 2-methyl (2S)-6-hydroxypiperidine-1,2-dicarboxylate (266 g, 1.03 mol, 1.00 eq) in MeOH (1.33 L) was added TsOH·H₂O (39.1 g, 205 mmol, 0.20 eq). The reaction mixture was stirred at 25° C. for 12 h. Two batches of equal scale were performed. The reaction batches were combined and concentrated under in vacuo. The crude residue was dissolved in EtOAc (3.00 L) and washed with saturated aqueous NaHCO₃ solution (2.00 L×2). The combined organic layers were washed with brine (1.50 L×2), dried over Na₂SO₄, filtered, and concentrated under reduced pressure. The resulting residue was purified by column chromatography (petroleum ether/EtOAc=100/0 to 20/1) to give 1-(tert-butyl) 2-methyl (2S)-6-methoxypiperidine-1,2-dicarboxylate (427 g) as yellow oil. ¹H NMR (400 MHz, CDCl₃) δ 5.33 (m, 1H), 4.74 (m, 1H), 3.71 (s, 3H), 3.30 (s, 3H), 2.33-2.25 (m, 1H), 1.92-1.81 (m, 2H), 1.69-1.58 (m, 2H), 1.50 (s, 9H), 1.47 (s, 1H).

### Step 5: Preparation of 1-(tert-butyl) 2-methyl (2S)-6-allylpiperidine-1,2-dicarboxylate

To a solution of 1-(tert-butyl) 2-methyl (2S)-6-methoxypiperidine-1,2-dicarboxylate (136 g, 496 mmol, 1.00 eq) in CH₂Cl₂ (1.35 L) was added allyltrimethylsilane (113 g, 992 mmol, 158 mL, 2.00 eq). The reaction mixture was cooled to −78° C., followed by dropwise addition of BF₃·Et₂O (76.1 g, 536 mmol, 66.1 mL, 1.08 eq) and stirred for 1 h. Three batches of equal scale were performed. The reaction mixtures were combined and quenched by pouring into H₂O (10.0 L). The reaction mixture was extracted with CH₂Cl₂ (1.00 L×2). Combined organic layers were washed with brine (4.00 L×2), dried over Na₂SO₄, filtered, and concentrated to give 1-(tert-butyl) 2-methyl (2S)-6-allylpiperidine-1,2-dicarboxylate (334 g) as yellow oil.

### Step 6: Preparation of 1-(tert-butyl) 2-methyl (2S)-6-allylpiperidine-1,2-dicarboxylate

To a cooled (0° C.) solution of 1-(tert-butyl) 2-methyl (2S)-6-allylpiperidine-1,2-dicarboxylate (188 g, 662 mmol, 1.00 eq) in MeOH (938 mL) was added a solution of 4 M HCl in 1,4-dioxane (827 mL, 5.00 eq). The mixture was warmed to 25° C. and stirred for 2 h. Two batches of equal scale were performed, and the mixtures were combined and concentrated under vacuum. The resulting residue was suspended in EtOAc (5.00 L) and H₂O (2.50 L). The aqueous layer was removed, and the organic layer was washed with saturated aqueous NaHCO₃ solution (5.00 L×2). The organic layer was dried over Na₂SO₄, filtered, and concentrated under reduced pressure to give 1-(tert-butyl) 2-methyl (2S)-6-allylpiperidine-1,2-dicarboxylate (76.6 g) as yellow oil. ¹H NMR (400 MHz, CDCl₃) δ 5.81-5.72 (m, 1H), 5.15-5.07 (m, 2H), 3.71 (s, 3H), 3.35-3.32 (m, 1H), 2.58-2.53 (m, 1H), 2.24-2.14 (m, 3H), 2.12-2.00 (m, 1H), 1.91-1.84 (m, 1H), 1.64 (d, J=13.2 Hz, 1H), 1.45-1.34 (m, 2H), 1.14-1.07 (m, 1H).

### Step 7: Preparation of Methyl (2S)-6-allyl-1-((S)-2-((tert-butoxycarbonyl)amino)pent-4-enoyl)piperidine-2-carboxylate

To a solution of 1-(tert-butyl) 2-methyl (2S)-6-allylpiperidine-1,2-dicarboxylate (56.5 g, 308 mmol, crude purity, 1.00 eq) in THF (1.41 L) was added (S)-2-((tert-butoxycarbonyl)amino)pent-4-enoic acid (67.7 g, 314 mmol, 1.02 eq) and IIDQ (100 g, 330 mmol, 1.07 eq). The mixture was stirred for 12 h and subsequently diluted with EtOAc (2.80 L). The organic layer was washed with aqueous 1 N HCl (2.80 L), followed by saturated aqueous NaHCO₃(2.80 L). The combined organic layers were washed with brine (2.80 L), dried over Na₂SO₄, filtered and concentrated. The residue was purified by column chromatography (petroleum ether/EtOAc=100/0 to 5/1) to give methyl (2S)-6-allyl-1-((S)-2-((tert-butoxycarbonyl)amino)pent-4-enoyl)piperidine-2-carboxylate (22.4 g, 54.4 mmol, 4.7% yield over five steps, 92.6% purity) as light yellow oil. LCMS (ESI) m/z=381.3 [M+H]⁺.

The intermediate shown in Table 9 was synthesized using the procedure described above and starting from 1-(tert-butyl) 2-methyl (2S)-6-allylpiperidine-1,2-dicarboxylate, appropriate N-protected amino acid [(S)-2-((tert-butoxycarbonyl)amino)pent-4-enoic acid], IIDQ, and THF.

TABLE 9

| Name | Structure | LCMS |
|---|---|---|
| methyl (2S)-6- allyl-1-((S)-2-((tert- butoxycarbonyl) amino)but-3- enoyl)piperidine- 2-carboxylate | 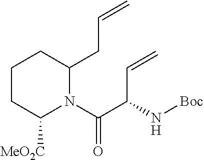 [SMILES: C=CCC1CCC[C@@H](C(=O)OC)N1C(=O)[C@H](C=C)NC(=O)OC(C)(C)C] | 367.3 [M + H]⁺ |

### Step 8: Preparation of Methyl (4S,7S,11aR,Z)-7-((tert-butoxycarbonyl)amino)-6-oxo-1,3,4,6,7,8,11,11a-octahydro-2H-pyrido[1,2-a]azocine-4-carboxylate

To a solution of methyl (2S)-6-allyl-1-((S)-2-((tert-butoxycarbonyl)amino)pent-4-enoyl)piperidine-2-carboxylate (21.8 g, 53.0 mmol, 92.6% purity, 1.00 eq) in CH₂Cl₂ (1.09 L) was added 1st generation Grubb's catalyst (8.72 g, 10.6 mmol, 0.20 eq). The mixture was heated to 50° C. After 5 h, LCMS analysis showed the reaction contained ˜14% of starting material. To the mixture was introduced additional 1st generation Grubb's catalyst (8.72 g, 10.6 mmol, 0.20 eq) and the mixture was allowed to age for another 2 h. The reaction mixture was cooled to room temperature and concentrated to give a residue. The residue was purified by column chromatography (petroleum ether/EtOAc=100/1 to 20/1) to give methyl (4S,7S,11aR,Z)-7-((tert-butoxycarbonyl)amino)-6-oxo-1,3,4,6,7,8,11,11a-octahydro-2H-pyrido[1,2-a]azocine-4-carboxylate (10.0 g) as brown oil. LCMS (ESI) m/z=353.3 [M+H]⁺.

The intermediate shown in Table 10 was synthesized via ring closing metathesis using the conditions described above for the preparation of methyl (4S,7S,11aR,Z)-7-((tert-butoxycarbonyl)amino)-6-oxo-1,3,4,6,7,8,11,11a-octahydro-2H-pyrido[1,2-a]azocine-4-carboxylate and starting from methyl (2S)-6-allyl-1-((S)-2-((tert-butoxycarbonyl)amino)but-3-enoyl)piperidine-2-carboxylate and using 1st generation Grubb's catalyst.

TABLE 10

| Name | Structure | LCMS |
|---|---|---|
| methyl (4S,7S,10aR)- 7-((tert- butoxycarbonyl) amino)-6-oxo- 1,2,3,4,6,7,10,10a- octahydropyrido[1,2- a]azepine-4-carboxylate | 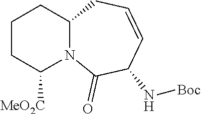 [SMILES: COC(=O)[C@@H]1CCC[C@@H]2CC=C[C@H](NC(=O)OC(C)(C)C)C(=O)N21] | 239.2 [(M − 100) + H]⁺ |

### Step 9: Preparation of Methyl (4S,7S,11aS)-7-((tert-butoxycarbonyl)amino)-6-oxodecahydro-2H-pyrido[1,2-a]azocine-4-carboxylate

To a solution of methyl (4S,7S,11aR,Z)-7-((tert-butoxycarbonyl)amino)-6-oxo-1,3,4,6,7,8,11,11a-octahydro-2H-pyrido[1,2-a]azocine-4-carboxylate (10.4 g, 29.5 mmol, 1.00 eq) in EtOAc (100 mL) was added 10% Pd/C (2.08 g) under N₂ (g). The suspension was subjected to three cycles of degassing under vacuum and purging with H₂ (g). The mixture was stirred at 25° C. under H₂ (g) (50 psi). After 12 h, the mixture was filtered and the filtrate was concentrated in vacuo. The residue was purified by prep-HPLC (Phenomenex luna C18 250*80 mm*10 um; mobile phase: [water (TFA)-acetonitrile]; B %: 50%-80%). The mixture was adjusted pH to 7-8 with aqueous saturated NaHCO₃ solution and carefully concentrated under reduced pressure to remove acetonitrile. The aqueous layer was extracted with EtOAc (250 mL×2). The combined organic layers were washed with brine (200 mL), dried over Na₂SO₄, filtered and concentrated under reduced pressure to give methyl (4S,7S,11aS)-7-((tert-butoxycarbonyl)amino)-6-oxodecahydro-2H-pyrido[1,2-a]azocine-4-carboxylate (6.46 g, 18.1 mmol, 31.2% yield over two steps) as dark brown gum. LCMS (ESI) m/z=355.2 [M+H]⁺; ¹H NMR (400 MHz, CDCl₃) δ 5.82 (d, J=7.2 Hz, 1H), 5.20 (t, J=2.4 Hz, 1H), 4.67-4.62 (m, 1H), 4.24-4.21 (m, 1H), 3.72 (s, 3H), 3.31-3.28 (m, 1H), 2.14-2.10 (m, 1H), 2.03-1.65 (m, 6H), 1.60-1.36 (m, 5H), 1.44 (s, 9H), 1.27-1.21 (m, 1H).

The intermediate shown in Table 11 was synthesized using the hydrogenation conditions described above for the preparation of methyl (4S,7S,11aS)-7-((tert-butoxycarbonyl)amino)-6-oxodecahydro-2H-pyrido[1,2-a]azocine-4-carboxylate and starting from methyl (4S,7S,10aR)-7-((tert-butoxycarbonyl)amino)-6-oxo-1,2,3,4,6,7,10,10a-octahydropyrido[1,2-a]azepine-4-carboxylate.

TABLE 11

| Name | Structure | LCMS |
|---|---|---|
| methyl (4S,7S,10aS)- 7-((tert-butoxy- carbonyl)amino)-6- oxodecahydropyrido [1,2-a]azepine-4- carboxylate | 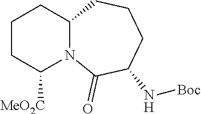 [SMILES: COC(=O)[C@@H]1CCC[C@@H]2CCC[C@H](NC(=O)OC(C)(C)C)C(=O)N21] | 241.2 [M + H]⁺ |

### Step 10: Preparation of (4S,7S,11aS)-7-((tert-butoxycarbonyl)amino)-6-oxodecahydro-2H-pyrido[1,2-a]azocine-4-carboxylic acid

(4S,7S,11aS)-7-((tert-butoxycarbonyl)amino)-6-oxodecahydro-2H-pyrido[1,2-a]azocine-4-carboxylic acid was prepared using the hydrolysis conditions described previously in Step 4 for the preparation (3S,6S,10aS)-6-((tert-butoxycarbonyl)amino)-5-oxodecahydropyrrolo[1,2-a]azocine-3-carboxylic acid.

The intermediate shown in Table 12 was synthesized using the conditions described in Step 4 for the preparation (3S,6S,10aS)-6-((tert-butoxycarbonyl)amino)-5-oxodecahydropyrrolo[1,2-a]azocine-3-carboxylic acid.

TABLE 12

| Name | Structure | LCMS |
|---|---|---|
| (4S,7S,10aS)-7-((tert- butoxycarbonyl)amino)-6- oxodecahydropyrido[1,2- a]azepine-4-carboxylic acid | 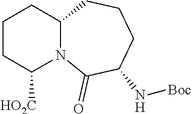 [SMILES: CC(C)(C)OC(=O)N[C@H]1CCC[C@H]2CCC[C@@H](C(=O)O)N2C1=O] | 327.3 [M + H]⁺ |

### Synthesis of (1R,3S,6S,11aS)-6-((tert-butoxycarbonyl)amino)-1-hydroxy-5-oxodecahydro-1H-pyrrolo[1,2-a]azonine-3-carboxylic acid and (3S,6S,11aS)-6-((tert-butoxycarbonyl)amino)-5-oxodecahydro-1H-pyrrolo[1,2-a]azonine-3-carboxylic acid

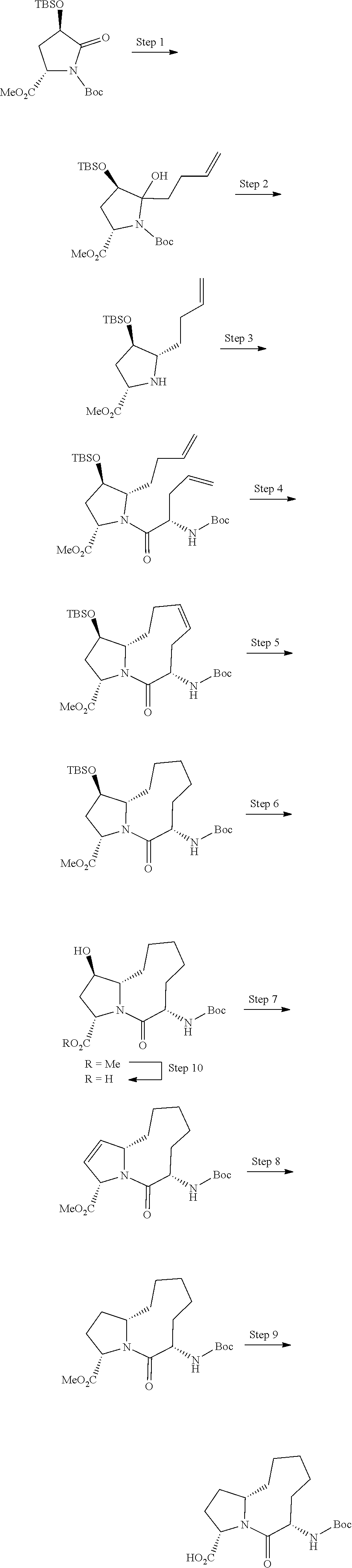

### Preparation of 1-(tert-butyl) 2-methyl (2S,4R)-4-((tert-butyldimethylsilyl)oxy)-5-oxopyrrolidine-1,2-dicarboxylate

1-(tert-butyl) 2-methyl (2S,4R)-4-((tert-butyldimethylsilyl)oxy)-5-oxopyrrolidine-1,2-dicarboxylate was prepared according the protocol reported in WO2015010626.

### Step 1: Preparation of 1-(tert-butyl) 2-methyl (2S,4R)-5-(but-3-en-1-yl)-4-((tert-butyldimethylsilyl)oxy)-5-hydroxypyrrolidine-1,2-dicarboxylate

To a cooled (−78° C.) solution of 1-(tert-butyl) 2-methyl (2S,4R)-4-((tert-butyldimethylsilyl)oxy)-5-oxopyrrolidine-1,2-dicarboxylate (165 g, 442 mmol, 1.00 eq) in THF (1.50 L) was added a 1 M solution of but-3-en-1-ylmagnesium bromide in THF (552 mL, 1.25 eq). The reaction mixtures were stirred for 1 h. Two batches of equal scale were performed in parallel and combined for work-up. The reaction mixtures were poured into saturated aqueous NH₄Cl (6.00 L) and extracted with EtOAc (5.00 L×3). The combined organic layers were washed with brine (5.00 L), dried over Na₂SO₄, filtered, and concentrated to give 1-(tert-butyl) 2-methyl (2S,4R)-5-(but-3-en-1-yl)-4-((tert-butyldimethylsilyl)oxy)-5-hydroxypyrrolidine-1,2-dicarboxylate (357 g) as yellow oil. ¹H NMR (400 MHz, CDCl₃) δ 5.95-5.72 (m, 1H), 5.13-4.92 (m, 2H), 4.53-4.39 (m, 1H), 4.18 (t, J=5.2 Hz, 1H), 3.79-3.71 (m, 3H), 2.84-2.70 (m, 1H), 2.63-2.54 (m, 1H), 2.38-2.24 (m, 2H), 2.22-2.08 (m, 2H), 1.47-1.39 (m, 9H), 0.99-0.90 (m, 9H), 0.18-0.12 (m, 3H), 0.11-0.06 (m, 3H).

### Step 2: Preparation of Methyl (2S,4R,5S)-5-(but-3-en-1-yl)-4-((tert-butyldimethylsilyl)oxy)pyrrolidine-2-carboxylate

To a cooled (0° C.) solution of 1-(tert-butyl) 2-methyl (2S,4R)-5-(but-3-en-1-yl)-4-((tert-butyldimethylsilyl)oxy)-5-hydroxypyrrolidine-1,2-dicarboxylate (178 g, 414 mmol, 1.00 eq) in CH₂Cl₂ (1.80 L) was sequentially added TFA (472 g, 4.14 mol, 307 mL, 10.0 eq), followed by Et₃SiH (241 g, 2.07 mol, 331 mL, 5.00 eq). The mixture was stirred 3 h while maintaining the reaction temperature between 0° C. to 25° C. Two batches of equal scale were performed in parallel and combined during work-up. The combined reaction mixtures were concentrated to give a residue. The resultant residue was dissolved with EtOAc (2.00 L) and saturated aqueous NaHCO₃ was added until the aqueous layer was basic (pH ˜8). The mixture was extracted with EtOAc (2.00 L×3). The combined organic layers were washed with brine (2.00 L), dried over Na₂SO₄, filtered, and concentrated. The residue was purified by column chromatography (petroleum ether/EtOAc=1/0-10/1) to give methyl (2S,4R,5S)-5-(but-3-en-1-yl)-4-((tert-butyldimethylsilyl)oxy)pyrrolidine-2-carboxylate (102 g, 325 mmol, 39.3% yield for four steps) as yellow oil. ¹H NMR (400 MHz, CDCl₃) δ 5.89-5.79 (m, 1H), 5.08-5.03 (m, 1H), 5.00-4.97 (m, 1H), 3.97 (t, J=8.0 Hz, 1H), 3.89-3.84 (m, 1H), 3.74 (s, 3H), 2.94-2.90 (m, 1H), 2.24-2.11 (m, 3H), 2.04-1.96 (m, 2H), 1.73-1.61 (m, 1H), 1.51-1.42 (m, 1H), 0.89 (s, 9H), 0.06 (d, J=1.0 Hz, 6H).

### Step 3: Preparation of Methyl (2S,4R,5S)-5-(but-3-en-1-yl)-1-((S)-2-((tert-butoxycarbonyl)amino)pent-4-enoyl)-4-((tert-butyldimethylsilyl)oxy)pyrrolidine-2-carboxylate

To a solution of methyl (2S,4R,5S)-5-(but-3-en-1-yl)-4-((tert-butyldimethylsilyl)oxy)pyrrolidine-2-carboxylate in DMF (1.00 L) was sequentially added N,N-diisopropylethylamine (283 mL, 1.63 mol, 5.00 eq), (S)-2-((tert-butoxycarbonyl)amino)pent-4-enoic acid (77.0 g, 358 mmol, 1.10 eq) and HATU (247 g, 651 mmol, 2.00 eq). The mixture was stirred at for 3 h at ambient temperatures and subsequently poured into water (2.00 L). The mixture was extracted with EtOAc (1.00 L×3). The combined organic layers were washed with saturated aqueous NaHCO₃(1.00 L×3), saturated aqueous NH₄Cl (1.00 L), brine (1.00 L), dried over Na₂SO₄, filtered, and concentrated. The residue was purified by column chromatography (petroleum ether/EtOAc=1/0-10/1) to give methyl (2S,4R,5S)-5-(but-3-en-1-yl)-1-((S)-2-((tert-butoxycarbonyl)amino)pent-4-enoyl)-4-((tert-butyldimethylsilyl)oxy)pyrrolidine-2-carboxylate (121 g, 220 mmol, 67.5% yield, 92.7% purity) as yellow oil. LCMS (ESI) m/z=511.2 [M+H]⁺; ¹H NMR (400 MHz, CDCl₃) δ 5.91-5.66 (m, 2H), 5.18-5.00 (m, 4H), 4.66-4.56 (m, 1H), 4.50-4.41 (m, 1H), 4.24 (d, J=3.2 Hz, 1H), 4.16-4.04 (m, 1H), 3.98-3.95 (m, 1H), 3.80-3.71 (m, 3H), 2.58-2.48 (m, 1H), 2.43-2.27 (m, 2H), 2.24-2.13 (m, 3H), 2.09-2.01 (m, 1H), 1.85-1.75 (m, 1H), 1.44-1.39 (m, 9H), 0.89-0.82 (m, 9H), 0.09-0.04 (m, 6H).

### Step 4: Preparation of Methyl (1R,3S,6S,11aS,Z)-6-((tert-butoxycarbonyl)amino)-1-((tert-butyldimethylsilyl)oxy)-5-oxo-2,3,5,6,7,10,11,11a-octahydro-1H-pyrrolo[1,2-a]azonine-3-carboxylate

To a solution of methyl (2S,4R,5S)-5-(but-3-en-1-yl)-1-((S)-2-((tert-butoxycarbonyl)amino)pent-4-enoyl)-4-((tert-butyldimethylsilyl)oxy)pyrrolidine-2-carboxylate (60.5 g, 118 mmol, 1.00 eq) in CH₂Cl₂ (1.20 L) was added 1^{st} generation Grubb's catalyst (9.75 g, 11.9 mmol, 0.10 eq) under N₂ (g). The mixture was heated to 55° C. and stirred for 12 h. Two batches of equal scale were performed in parallel and were combined for work-up. The combined reaction mixtures were cooled to ambient temperatures and concentrated to give a residue. The residue was purified by column chromatography (petroleum ether/EtOAc=1/0-10/1) to give methyl (1R,3S,6S,11aS,Z)-6-((tert-butoxycarbonyl)amino)-1-((tert-butyldimethylsilyl)oxy)-5-oxo-2,3,5,6,7,10,11,11a-octahydro-1H-pyrrolo[1,2-a]azonine-3-carboxylate (70.8 g, 130 mmol, 54.9% yield, 88.6% purity) as brown oil. LCMS (ESI) m/z=483.2 [M+H]⁺; ¹H NMR (400 MHz, CDCl₃) δ 6.02-5.90 (m, 1H), 5.83 (d, J=6.8 Hz, 1H), 5.71-5.49 (m, 1H), 4.78 (t, J=9.0 Hz, 1H), 4.34 (t, J=6.8 Hz, 1H), 4.10-4.07 (m, 1H), 3.88-3.82 (m, 1H), 3.75 (s, 3H), 2.66-2.58 (m, 1H), 2.51-2.38 (m, 1H), 2.35-2.30 (m, 1H), 2.27-2.18 (m, 1H), 2.16-2.09 (m, 2H), 1.54-1.46 (m, 2H), 1.42 (s, 9H), 0.80 (s, 9H), 0.09-0.03 (m, 6H).

### Step 5: Preparation of Methyl (1R,3S,6S,11aS)-6-((tert-butoxycarbonyl)amino)-1-((tert-butyldimethylsilyl)oxy)-5-oxodecahydro-1H-pyrrolo[1,2-a]azonine-3-carboxylate

To a solution of methyl (1R,3S,6S,11aS,Z)-6-((tert-butoxycarbonyl)amino)-1-((tert-butyldimethylsilyl)oxy)-5-oxo-2,3,5,6,7,10,11,11a-octahydro-1H-pyrrolo[1,2-a]azonine-3-carboxylate (70.8 g, 147 mmol, 1.00 eq) in EtOH (700 mL) was added Pd/C (7.00 g, 10% wt) under a constant stream of N₂ (g). The reaction mixture was degassed under vacuum and purged with H₂ (g) (3×). The reaction mixture was stirred under H₂ (g) (50 psi) for 12 h. The reaction mixture was filtered and the filter cake was washed with EtOH (200 mL×3). The filtrate was concentrated to give a methyl (1R,3S,6S,11aS)-6-((tert-butoxycarbonyl)amino)-1-((tert-butyldimethylsilyl)oxy)-5-oxodecahydro-1H-pyrrolo[1,2-a]azonine-3-carboxylate (65.0 g, 125 mmol, 85.5% yield, 93.5% purity) as an off-white solid. LCMS (ESI) m/z=485.1 [M+H]⁺; ¹H NMR (400 MHz, CDCl₃) δ 5.17 (d, J=8.8 Hz, 1H), 4.86-4.76 (m, 1H), 4.70-4.65 (m, 1H), 4.10 (d, J=2.0 Hz, 1H), 3.86 (d, J=10.0 Hz, 1H), 3.77 (s, 3H), 2.20-2.15 (m, 1H), 2.13-2.03 (m, 1H), 2.02-1.94 (m, 1H), 1.88-1.73 (m, 5H), 1.59 (s, 2H), 1.54-1.44 (m, 2H), 1.41 (s, 9H), 0.85 (s, 9H), 0.08 (d, J=5.0 Hz, 6H).

### Step 6: Preparation of Methyl (1R,3S,6S,11aS)-6-((tert-butoxycarbonyl)amino)-1-hydroxy-5-oxodecahydro-1H-pyrrolo[1,2-a]azonine-3-carboxylate

A cooled (−10° C.) solution of methyl (1R,3S,6S,11aS)-6-((tert-butoxycarbonyl)amino)-1-((tert-butyldimethylsilyl)oxy)-5-oxodecahydro-1H-pyrrolo[1,2-a]azonine-3-carboxylate (65.0 g, 134 mmol, 1.00 eq) in THF (650 mL) was added in a dropwise manner a 1 M solution of TBAF in THF (268 mL, 2.00 eq). The reaction mixture was stirred at 0° C. for 1 h and subsequently poured into water (1.50 L). The mixture was extracted with EtOAc (1.00 L×2). The combined organic layers were washed with brine (1.00 L), dried over Na₂SO₄, filtered, and concentrated to give a residue. The residue was purified by column chromatography (petroleum ether/EtOAc=1/0-3/2) to give methyl (1R,3S,6S,11aS)-6-((tert-butoxycarbonyl)amino)-1-hydroxy-5-oxodecahydro-1H-pyrrolo[1,2-a]azonine-3-carboxylate (40.8 g, 107 mmol, 79.9% yield, 97.3% purity) as an off-white solid. LCMS (ESI) m/z=371.4 [M+H]⁺; ¹H NMR (400 MHz, CDCl₃) δ 5.22 (d, J=8.0 Hz, 1H), 4.74-4.70 (m, 2H), 4.19 (d, J=3.2 Hz, 1H), 4.01 (d, J=10.0 Hz, 1H), 3.76 (s, 3H), 2.78 (s, 1H), 2.34-2.28 (m, 1H), 2.12-1.97 (m, 2H), 1.89-1.83 (m, 2H), 1.79-1.74 (m, 3H), 1.61-1.57 (m, 4H), 1.42 (s, 9H).

### Step 7: Preparation of Methyl (3S,6S,11aS)-6-((tert-butoxycarbonyl)amino)-5-oxo-5,6,7,8,9,10,11,11a-octahydro-3H-pyrrolo[1,2-a]azonine-3-carboxylate

To a cooled (−78° C.) solution of methyl (1R,3S,6S,11aS)-6-((tert-butoxycarbonyl)amino)-1-hydroxy-5-oxodecahydro-1H-pyrrolo[1,2-a]azonine-3-carboxylate (12.0 g, 32.4 mmol, 1.00 eq) in CH₂Cl₂ (120 mL) was slowly added DAST (25.7 mL, 194 mmol, 6.00 eq) under N₂ (g). After complete addition of DAST, the mixture was allowed to warm to 25° C. and stirred for 8 h. The reaction mixture was cooled to at 0° C. and subsequently quenched by addition of saturated aqueous NaHCO₃ (300 mL). The biphasic mixture was extracted with CH₂Cl₂ (200 mL×3). The combined organic layers were washed with brine (200 mL), dried over Na₂SO₄, filtered, and concentrated to give a residue. The residue was purified by column chromatography (petroleum ether/EtOAc=1/0-3/1) to give methyl (3S,6S,11aS)-6-((tert-butoxycarbonyl)amino)-5-oxo-5,6,7,8,9,10,11,11a-octahydro-3H-pyrrolo[1,2-a]azonine-3-carboxylate (3.00 g, 8.36 mmol, 25.8% yield, 98.2% purity) as a white solid. LCMS (ESI) m/z=353.1 [M+H]⁺; ¹H NMR (400 MHz, CDCl₃) δ 5.87-5.78 (m, 2H), 5.43 (s, 1H), 5.18 (d, J=8.0 Hz, 1H), 4.88-4.74 (m, 2H), 3.77 (s, 3H), 2.04-1.93 (m, 2H), 1.89-1.76 (m, 4H), 1.63-1.55 (m, 4H), 1.43 (s, 9H).

### Step 8: Preparation of Methyl (3S,6S,11aS)-6-((tert-butoxycarbonyl)amino)-5-oxodecahydro-1H-pyrrolo[1,2-a]azonine-3-carboxylate

To a solution of methyl (3S,6S,11aS)-6-((tert-butoxycarbonyl)amino)-5-oxo-5,6,7,8,9,10,11,11a-octahydro-3H-pyrrolo[1,2-a]azonine-3-carboxylate (3.00 g, 8.51 mmol, 1.00 eq) in MeOH (30.0 mL) was added Pd/C (0.60 g, 10% wt) under a constant stream of N₂ (g). The mixture was degassed under vacuum and purged with H₂ (g) (15 psi) (3×). The mixture was heated to 45° C. and stirred at under H₂ (g) (15 psi) for 22 h. The mixture was filtered, and the filter cake was washed with MeOH (50.0 mL×3). The filtrate was concentrated to give a residue. The residue was dissolved with acetonitrile (5.00 mL) and H₂O (30.0 mL) and subsequently lyophilized to give methyl (3S,6S,11aS)-6-((tert-butoxycarbonyl)amino)-5-oxodecahydro-1H-pyrrolo[1,2-a]azonine-3-carboxylate (5.72 g, 15.9 mmol, 94.2% yield, 98.2% purity) as an off-white solid. LCMS (ESI) m/z=355.1 [M+H]⁺; ¹H NMR (400 MHz, CDCl₃) δ 5.16 (d, J=8.8 Hz, 1H), 4.89-4.78 (m, 1H), 4.60-4.51 (m, 1H), 4.22-4.13 (m, 1H), 3.76 (s, 3H), 2.30-2.18 (m, 2H), 2.01 (s, 2H), 1.83-1.73 (m, 7H), 1.60-1.55 (m, 3H), 1.43 (s, 9H).

### Step 9: Preparation of (3S,6S,11aS)-6-((tert-butoxycarbonyl)amino)-5-oxodecahydro-1H-pyrrolo[1,2-a]azonine-3-carboxylic acid

To a cooled (0° C.) solution of methyl (3S,6S,11aS)-6-((tert-butoxycarbonyl)amino)-5-oxodecahydro-1H-pyrrolo[1,2-a]azonine-3-carboxylate (1.00 g, 2.82 mmol, 1.00 eq) in THF (10 mL) was added LiOH—H₂O (153 mg, 3.66 mmol, 1.3 eq) and water (10 mL). The mixture was stirred for 16 h, and subsequently acidified with NaHSO₄. The aqueous layer was extracted with EtOAc (3×). The combined organic layers were dried over Na₂SO₄ and concentrated to give (3S,6S,11aS)-6-((tert-butoxycarbonyl)amino)-5-oxodecahydro-1H-pyrrolo[1,2-a]azonine-3-carboxylic acid (855 mg, 2.51 mmol, 89.1% yield) as a solid. LCMS (ESI) m/z=341.4 [M+H]⁺.

### Step 10: Preparation of Methyl (1R,3S,6S,11aS)-6-((tert-butoxycarbonyl)amino)-1-hydroxy-5-oxodecahydro-1H-pyrrolo[1,2-a]azonine-3-carboxylate

Methyl (1R,3S,6S,11aS)-6-((tert-butoxycarbonyl)amino)-1-hydroxy-5-oxodecahydro-1H-pyrrolo[1,2-a]azonine-3-carboxylate was prepared using the hydrolysis procedure described above for the preparation of (3S,6S,11aS)-6-((tert-butoxycarbonyl)amino)-5-oxodecahydro-1H-pyrrolo[1,2-a]azonine-3-carboxylic acid and starting from methyl (1R,3S,6S,11aS)-6-((tert-butoxycarbonyl)amino)-1-hydroxy-5-oxodecahydro-1H-pyrrolo[1,2-a]azonine-3-carboxylate. Starting from methyl (1R,3S,6S,11aS)-6-((tert-butoxycarbonyl)amino)-1-hydroxy-5-oxodecahydro-1H-pyrrolo[1,2-a]azonine-3-carboxylate (500 mg, 1.34 mmol. 1.00 eq), LiOH—H₂O (107 mg, 2.68 mmol, 2.0 eq), THF (12 mL), and water (4 mL) afforded methyl (1R,3S,6S,11aS)-6-((tert-butoxycarbonyl)amino)-1-hydroxy-5-oxodecahydro-1H-pyrrolo[1,2-a]azonine-3-carboxylate (340 mg, 0.953 mmol, 71.2% yield) as solids. LCMS (ESI) m/z=357.3 [M+H]⁺.

### Synthesis of (3S,6S,9R,10aR)-6-{[(tert-butoxy)carbonyl]amino}-9-hydroxy-5-oxo-decahydropyrrolo[1,2-a]azocine-3-carboxylic acid, (3S,6S,9S,10aR)-6-((tert-butoxycarbonyl)amino)-9-hydroxy-5-oxodecahydropyrrolo[1,2-a]azocine-3-carboxylic acid, (3S,6S,8S,10aR)-6-((tert-butoxycarbonyl)amino)-8-hydroxy-5-oxodecahydropyrrolo[1,2-a]azocine-3-carboxylic acid, (3S,6S,8R,10aR)-6-((tert-butoxycarbonyl)amino)-8-hydroxy-5-oxodecahydropyrrolo[1,2-a]azocine-3-carboxylic acid

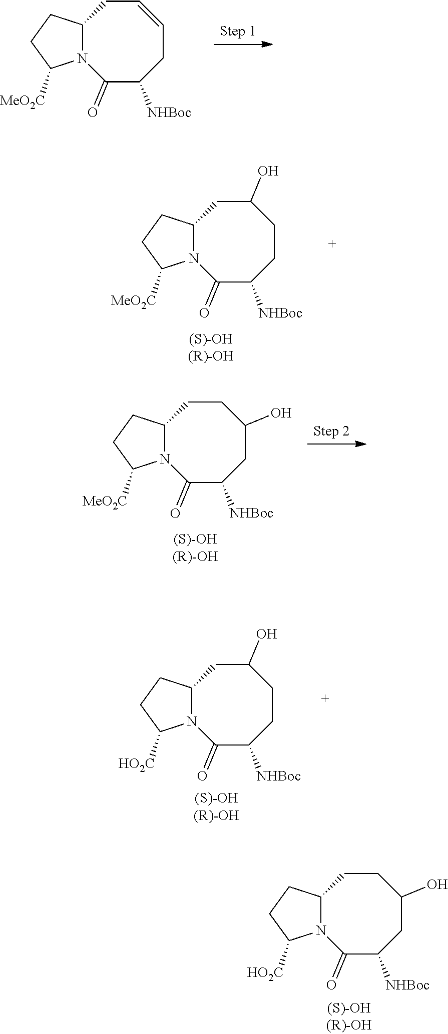

### Step 1: Preparation of Methyl (3S,6S,10aR)-6-((tert-butoxycarbonyl)amino)-9-hydroxy-5-oxodecahydropyrrolo[1,2-a]azocine-3-carboxylate and methyl (3S,6S,10aR)-6-((tert-butoxycarbonyl)amino)-8-hydroxy-5-oxodecahydropyrrolo[1,2-a]azocine-3-carboxylate

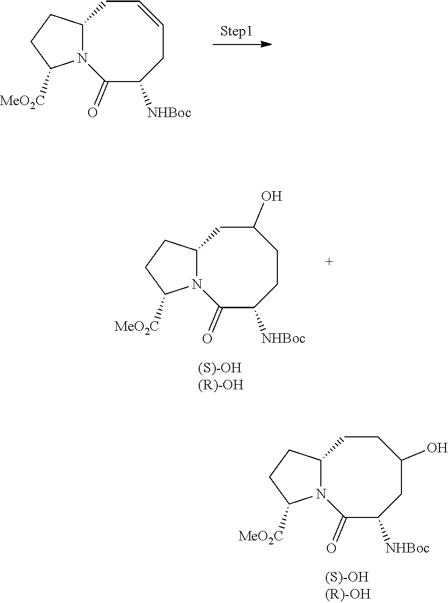

Methyl (3S,6S,10aR)-6-((tert-butoxycarbonyl)amino)-9-hydroxy-5-oxodecahydropyrrolo[1,2-a]azocine-3-carboxylate and methyl (3S,6S,10aR)-6-((tert-butoxycarbonyl)amino)-8-hydroxy-5-oxodecahydropyrrolo[1,2-a]azocine-3-carboxylate were prepared starting from methyl (3S,6S,10aR,Z)-6-((tert-butoxycarbonyl)amino)-5-oxo-1,2,3,5,6,7,10,10a-octahydropyrrolo[1,2-a]azocine-3-carboxylate and utilizing similar protocols established in Journal of Medicinal Chemistry (2010), 53(17), 6361-6367.

The mixture of alcohol diastereoisomers and regioisomers were separated by reverse phase chromatography [C18 cartridge eluting with a gradient of 5-40% MeOH in water (with 0.1% formic acid)] followed by chiral SFC separation [Lux i-Cellulose-5 21.2×250 mm 5 um column, column temp=40° C., flow rate 75 mL/min, 20% MeOH, cycle time: 5 min]. A representative reaction starting from methyl (3S,6S,10aR,Z)-6-((tert-butoxycarbonyl)amino)-5-oxo-1,2,3,5,6,7,10,10a-octahydropyrrolo[1,2-a]azocine-3-carboxylate (4.3 g, 12.7 mmol) afforded the following products:

Peak 1: Methyl (3S,6S,9R,10aR)-6-{[(tert-butoxy)carbonyl]amino}-9-hydroxy-5-oxo-decahydropyrrolo[1,2-a]azocine-3-carboxylate (760 mg, 2.13 mmol, 16.8% yield) as a white solid. LCMS (ESI) m/z=357.2 [M+H]⁺.

Peak 2: Methyl (3S,6S,9S,10aR)-6-{[(tert-butoxy)carbonyl]amino}-9-hydroxy-5-oxo-decahydropyrrolo[1,2-a]azocine-3-carboxylate (375 mg, 19% yield) as a clear thick oil. LCMS (ESI) m/z=357.2 [M+H]⁺.

Peak 3: Methyl (3S,6S,8R,10aR)-6-{[(tert-butoxy)carbonyl]amino}-8-hydroxy-5-oxo-decahydropyrrolo[1,2-a]azocine-3-carboxylate (795 mg, 40% yield) as a white foam. LCMS (ESI) m/z=357.2 [M+H]⁺.

Peak 4: Methyl (3S,6S,8S,10aR)-6-{[(tert-butoxy)carbonyl]amino}-8-hydroxy-5-oxo-decahydropyrrolo[1,2-a]azocine-3-carboxylate (600 mg, 30% yield) as a white solid. LCMS (ESI) m/z=357.2 [M+H]⁺.

### Step 2: Preparation of (3S,6S,9R,10aR)-6-{[(tert-Butoxy)carbonyl]amino}-9-hydroxy-5-oxo-decahydropyrrolo[1,2-a]azocine-3-carboxylic acid

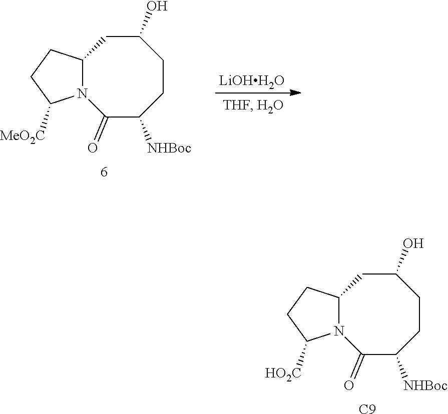

To a solution of methyl (3S,6S,9R,10aR)-6-{[(tert-butoxy)carbonyl]amino}-9-hydroxy-5-oxo-decahydropyrrolo[1,2-a]azocine-3-carboxylate (Peak 1) (1.08 g, 3.03 mmol, 1 eq) in a mixture of THF (24 mL) and water (8 mL) was added LiOH—H₂O (380 mg, 9.08 mmol, 3.0 eq). The reaction mixture was stirred at room temperature for 2 hours. The reaction was subsequently concentrated under reduced pressure to remove tetrahydrofuran. The crude residue was purified by reverse phase chromatography [C18 cartridge eluting with a gradient of 5-60% Acetonitrile in water (with 0.1% formic acid)] to give (3S,6S,9R,10aR)-6-{[(tert-butoxy)carbonyl]amino}-9-hydroxy-5-oxo-decahydropyrrolo[1,2-a]azocine-3-carboxylic acid (780 mg, 76% yield) as a white solid. LCMS (ESI) m/z=343.2 [M+H]⁺.

The following intermediates in Table 13 were prepared according to the general procedure described above starting from the appropriate starting materials.

TABLE 13

| Name | Structure | LCMS |
|---|---|---|
| (3S,6S,9S,10aR)-6-((tert- butoxycarbonyl)amino)-9-hydroxy- 5-oxodecahydropyrrolo[1,2- a]azocine-3-carboxylic acid | 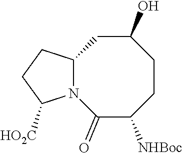 [SMILES: CC(C)(C)OC(=O)N[C@H]1CC[C@H](O)C[C@H]2CC[C@@H](C(=O)O)N2C1=O] | 343.2 [M + H]⁺ |
| | Peak 2 of starting material | |
| | building block used for synthesis | |
| (3S,6S,8S,10aR)-6-((tert- butoxycarbonyl)amino)-8-hydroxy- 5-oxodecahydropyrrolo[1,2- a]azocine-3-carboxylic acid | 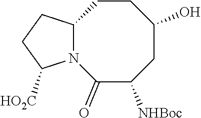 [SMILES: CC(C)(C)OC(=O)N[C@H]1C[C@@H](O)CC[C@H]2CC[C@@H](C(=O)O)N2C1=O] | 343.2 [M + H]⁺ |
| | Peak 4 of starting material | |
| | building block used for synthesis | |
| (3S,6S,8R,10aR)-6-((tert- butoxycarbonyl)amino)-8-hydroxy- 5-oxodecahydropyrrolo[1,2- a]azocine-3-carboxylic acid | 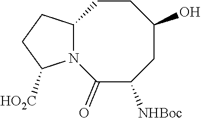 [SMILES: CC(C)(C)OC(=O)N[C@H]1C[C@H](O)CC[C@H]2CC[C@@H](C(=O)O)N2C1=O] | 343.2 [M + H]⁺ |
| | Peak 3 of starting material | |
| | building block used for synthesis | |

### Synthesis of (3S,6S,9S,10aR)-6-{[(tert-butoxy)carbonyl]amino}-9-hydroxy-9-methyl-5-oxo-decahydropyrrolo[1,2-a]azocine-3-carboxylic acid and (3S,6S,8R,10aR)-6-((tert-butoxycarbonyl)amino)-8-hydroxy-8-methyl-5-oxodecahydropyrrolo[1,2-a]azocine-3-carboxylic acid

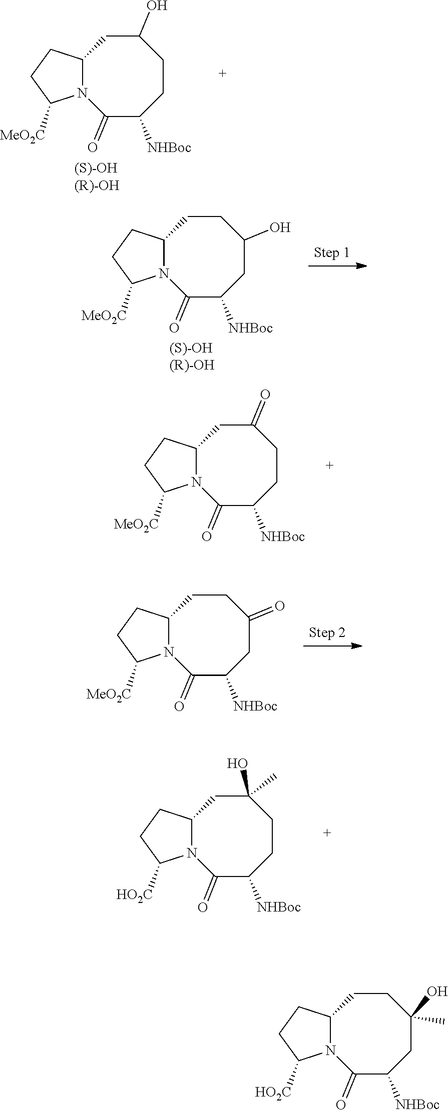

### Step 1: Preparation of Methyl (3S,6S,10aR)-6-{[(tert-butoxy)carbonyl]amino}-5,9-dioxo-decahydropyrrolo[1,2-a]azocine-3-carboxylate and methyl (3S,6S,10aR)-6-{[(tert-butoxy)carbonyl]amino}-5,8-dioxo-decahydropyrrolo[1,2-a]azocine-3-carboxylate

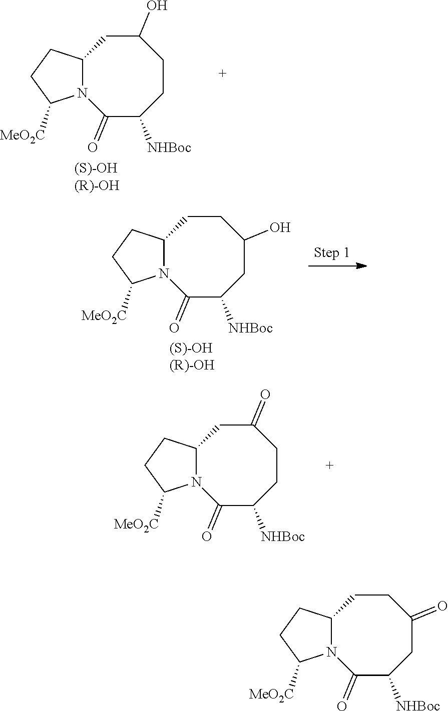

Methyl (3S,6S,10aR)-6-{[(tert-butoxy)carbonyl]amino}-5,9-dioxo-decahydropyrrolo[1,2-a]azocine-3-carboxylate and methyl (3S,6S,10aR)-6-{1[(tert-butoxy)carbonyl]amino}-5,8-dioxo-decahydropyrrolo[1,2-a]azocine-3-carboxylate were prepared using the protocol described in US 2009/0123480. The crude reaction mixture was purified by reverse phase chromatography using a [C18 cartridge eluting with a gradient of 5-100% acetonitrile in water (with 0.1% FA)] to give a mixture of ketone isomers (1.2 g) as a beige solid. The resultant ketone isomers were separated chiral SFC separation (Column Lux i-Cellulose-5 21.2×250 mm 5 um column, flow rate 75 mL/min, 15% MeOH) to give methyl (3S,6S,10aR)-6-{[(tert-butoxy)carbonyl]amino}-5,9-dioxo-decahydropyrrolo[1,2-a]azocine-3-carboxylate (9) (424 mg, 35.6% yield) as a white solid and methyl (3S,6S,10aR)-6-{[(tert-butoxy)carbonyl]amino}-5,8-dioxo-decahydropyrrolo[1,2-a]azocine-3-carboxylate (433 mg, 36.3% yield) as a white solid.

Methyl (3S,6S,10aR)-6-{[(tert-butoxy)carbonyl]amino}-5,9-dioxo-decahydropyrrolo[1,2-a]azocine-3-carboxylate: LCMS (ESI) m/z=299.1 [M+H]⁺; ¹H NMR: (400 MHz, CDCl₃) δ 5.27 (d, J=8.1 Hz, 1H), 4.59 (t, J=8.7 Hz, 1H), 4.45-4.31 (m, 2H), 3.80 (s, 3H), 3.20 (td, J=12.5, 4.9 Hz, 1H), 3.06 (t, J=12.0 Hz, 1H), 2.47-2.15 (m, 5H), 2.13-2.00 (m, 1H), 1.87 (dd, J=12.1, 7.0 Hz, 1H), 1.68-1.62 (m, 1H), 1.41 (s, 9H).

Methyl (3S,6S,10aR)-6-{[(tert-butoxy)carbonyl]amino}-5,8-dioxo-decahydropyrrolo[1,2-a]azocine-3-carboxylate: LCMS (ESI) m/z=299.1 [M+H]⁺; ¹H NMR: (400 MHz, CDCl₃) δ 5.56 (d, J=7.6 Hz, 1H), 5.13 (ddd, J=12.2, 7.6, 4.9 Hz, 1H), 4.51 (t, J=8.8 Hz, 1H), 4.33-4.23 (m, 1H), 3.73 (s, 3H), 3.07 (dd, J=13.8, 4.5 Hz, 1H), 2.96 (td, J=11.9, 2.9 Hz, 1H), 2.65-2.50 (m, 2H), 2.36-1.91 (m, 4H), 1.90-1.76 (m, 2H), 1.45 (s, 9H).

### Step 2: Preparation of (3S,6S,9S,10aR)-6-{[(tert-Butoxy)carbonyl]amino}-9-hydroxy-9-methyl-5-oxo-decahydropyrrolo[1,2-a]azocine-3-carboxylic acid

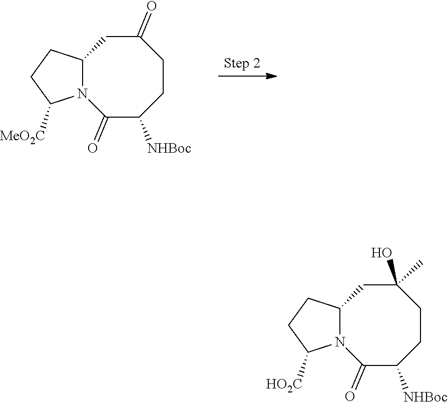 [SMILES: COC(=O)[C@@H]1CC[C@@H]2CC(=O)CC[C@H](NC(=O)OC(C)(C)C)C(=O)N21 ; CC(C)(C)OC(=O)N[C@H]1CC[C@](C)(O)C[C@H]2CC[C@@H](C(=O)O)N2C1=O]

To a cooled (−78° C.) solution of methyl (3S,6S,10aR)-6-{[(tert-butoxy)carbonyl]amino}-5,9-dioxo-decahydropyrrolo[1,2-a]azocine-3-carboxylate (250 mg, 705 μmol, 1 eq) in THF (15 mL) was added a 3 M solution of MeMgCl in THF (480 μL, 1.44 mmol, 2.05 eq). The reaction mixture was slowly warmed to 0° C. with stirring for 2 h and subsequently quenched with saturated aq. NH₄Cl. The product was extracted with EtOAc (2×) and the combined organic layers were dried with Na₂SO₄, filtered and concentrated in vacuo. The crude residue was purified by reverse phase chromatography [C18 cartridge eluting with a gradient of 5-60% Acetonitrile in water (with 0.1% formic acid)] to yield the resultant tertiary alcohol (240 mg, 647 μmol, 93.3%) as a white solid [as a 1:1 mixture of ketone and tertiary alcohol ratio based on ¹H NMR]. This mixture was dissolved in tetrahydrofuran (6 mL) and water (2 mL) and LiOH—H₂O (81.4 mg, 1.94 mmol, 3 eq) was then added. The reaction mixture was stirred for 4 h at room temperature. The reaction was concentrated under reduced pressure and purified by reverse phase chromatography [C₁₈ cartridge, elution gradient: 5-60% Acetonitrile in water (with 0.1% FA)]. The pure fractions were combined and evaporated to dryness to give (3S,6S,9S,10aR)-6-{[(tert-butoxy)carbonyl]amino}-9-hydroxy-9-methyl-5-oxo-decahydropyrrolo[1,2-a]azocine-3-carboxylic acid (82.0 mg, 230 μmol, 35.6% yield) as a white solid. LCMS (ESI) m/z=357.2 [M+H]⁺.

The following intermediate in Table 14 was prepared according to the protocol outlined above and starting from methyl (3S,6S,10aR)-6-{[(tert-butoxy)carbonyl]amino}-5,8-dioxo-decahydropyrrolo[1,2-a]azocine-3-carboxylate.

TABLE 14

| Name | Structure | LCMS |
|---|---|---|
| (3S,6S,8R,10aR)-6-((tert- butoxycarbonyl)amino)-8-hydroxy-8- methyl-5-oxodecahydropyrrolo[1,2- a]azocine-3-carboxylic acid | 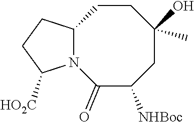 [SMILES: CC(C)(C)OC(=O)N[C@H]1C[C@](C)(O)CC[C@H]2CC[C@@H](C(=O)O)N2C1=O] | 357.2 [M + H]⁺ |

### Synthesis of (3S,6S,9S,10aR)-9-(azetidin-1-yl)-6-{[(tert-butoxy)carbonyl]amino}-5-oxo-decahydropyrrolo[1,2-a]azocine-3-carboxylic acid

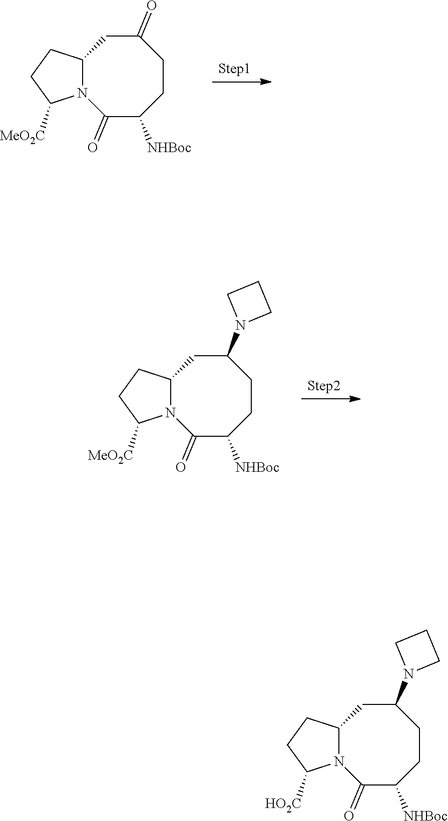 [SMILES: COC(=O)[C@@H]1CC[C@@H]2CC(=O)CC[C@H](NC(=O)OC(C)(C)C)C(=O)N21 ; COC(=O)[C@@H]1CC[C@@H]2C[C@@H](N3CCC3)CC[C@H](NC(=O)OC(C)(C)C)C(=O)N21 ; CC(C)(C)OC(=O)N[C@H]1CC[C@H](N2CCC2)C[C@H]2CC[C@@H](C(=O)O)N2C1=O]

### Step 1: Preparation of Methyl (3S,6S,9S,10aR)-9-(azetidin-1-yl)-6-((tert-butoxycarbonyl)amino)-5-oxodecahydropyrrolo[1,2-a]azocine-3-carboxylate

To a solution of methyl (3S,6S,10aR)-6-{[(tert-butoxy)carbonyl]amino}-5,9-dioxo-decahydropyrrolo[1,2-a]azocine-3-carboxylate (150 mg, 0.4232 mmol, 1 eq) and AcOH (26.5 μL, 465 μmol, 1.1 eq) in CH₂Cl₂ (10 mL) was added azetidine (55.7 μL, 846 μmol, 2 eq). The mixture was stirred for 2 h at room temperature. To the mixture was added NaBH(OAc)₃ (134 mg, 634 μmol, 1.5 eq) was added and the reaction was stirred for 1.5 h at room temperature. The reaction was quenched by the addition of a saturated aqueous solution of NaHCO₃(10 mL). The product was extracted with CH₂Cl₂ (3×). The combined organic layers were dried with Na₂SO₄, filtered and concentrated under reduced pressure to give crude methyl (3S,6S,9S,10aR)-9-(azetidin-1-yl)-6-((tert-butoxycarbonyl)amino)-5-oxodecahydropyrrolo[1,2-a]azocine-3-carboxylate as a white solid. LCMS (ESI) m/z=396.3 [M+H]⁺.

### Step 2: Preparation of (3S,6S,9S,10aR)-9-(azetidin-1-yl)-6-((tert-butoxycarbonyl)amino)-5-oxodecahydropyrrolo[1,2-a]azocine-3-carboxylic acid

To a solution of methyl (3S,6S,9S,10aR)-9-(azetidin-1-yl)-6-((tert-butoxycarbonyl)amino)-5-oxodecahydropyrrolo[1,2-a]azocine-3-carboxylate (180 mg, 0.4551 mmol) in THF (24 mL) and water (8 mL) was added LiOH—H₂O (57.0 mg, 1.36 mmol). The reaction was stirred for 4 h at room temperature. The reaction was then concentrated under reduced pressure. The crude residue was purified by reverse phase chromatography [C18 cartridge eluting with a gradient of 0-40% acetonitrile in water]. The pure fractions were concentrated to dryness and the product was subsequently lyophilized to give (3S,6S,9S,10aR)-9-(azetidin-1-yl)-6-((tert-butoxycarbonyl)amino)-5-oxodecahydropyrrolo[1,2-a]azocine-3-carboxylic acid (140 mg, 0.3669 mmol, 80.9% yield) as a white solid. LCMS (ESI) m/z=382.4 [M+H]⁺.

The following intermediate in Table 15 was prepared according to the procedure outlined above and starting from methyl (3S,6S,10aR)-6-{[(tert-butoxy)carbonyl]amino}-5,8-dioxo-decahydropyrrolo[1,2-a]azocine-3-carboxylate.

TABLE 15

| Name | Structure | LCMS |
|---|---|---|
| (3S,6S,8R,10aS)-8-(azetidin-1-yl)-6- ((tert-butoxycarbonyl)amino)-5- oxodecahydropyrrolo[1,2-a]azocine-3- carboxylic acid | 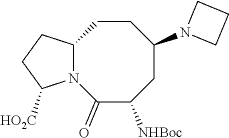 [SMILES: CC(C)(C)OC(=O)N[C@H]1C[C@H](N2CCC2)CC[C@H]2CC[C@@H](C(=O)O)N2C1=O] | 382.4 [M + H]⁺ |

### Synthesis of (3S,6S,10aR)-6-((tert-butoxycarbonyl)amino)-5-oxooctahydro-1H-spiro[cyclopropane-1,9-pyrrolo[1,2-a]azocine]-3-carboxylic acid

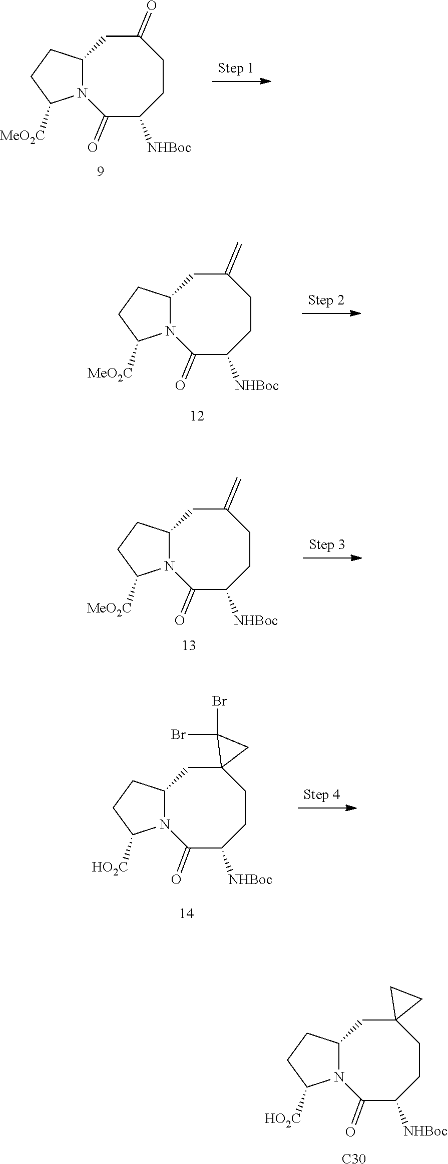

### Step 1: Preparation of Methyl (3S,6S,10aR)-6-{[(tert-butoxy)carbonyl]amino}-9-methylidene-5-oxo-decahydropyrrolo[1,2-a]azocine-3-carboxylate

To a cooled (0° C.) solution of methyltriphenylphosphonium bromide (628 mg, 1.76 mmol, 2.5 eq) in THF (10 mL) was added a solution of 1.0 M NaHMDS in THF (1.76 mL, 1.76 mmol, 2.5 eq). The resulting yellow suspension was warmed up to room temperature and stirred for 30 min. The yellow suspension was cooled to 0° C., followed by addition of a solution of methyl (3S,6S,10aR)-6-{[(tert-butoxy)carbonyl]amino}-5,9-dioxo-decahydropyrrolo[1,2-a]azocine-3-carboxylate (250 mg, 0.7054 mmol, 1 eq) in THF (10 mL). The resulting reaction mixture was stirred for 10 min and subsequently warmed to ambient temperatures over 1.5 h. The reaction mixture was quenched by the addition of brine (10 mL) and the product was extracted with EtOAc (30 mL×2). The combined organic layers were dried with Na₂SO₄, filtered, and concentrated in vacuo. The crude residue was purified by reverse phase chromatography [C₁₈ cartridge eluting with a gradient of 5-60% acetonitrile with 10 mM ammonium bicarbonate/ammonium hydroxide buffer pH=10] to give methyl (3S,6S,10aR)-6-{[(tert-butoxy)carbonyl]amino}-9-methylidene-5-oxo-decahydropyrrolo[1,2-a]azocine-3-carboxylate (210 mg, 0.5958 mmol, 84.6% yield) as a white solid. LCMS (ESI) m/z=353.2 [M+H]⁺.

### Step 2: Preparation of (3S,6S,10aR)-6-{[(tert-Butoxy)carbonyl]amino}-9-methylidene-5-oxo-decahydropyrrolo[1,2-a]azocine-3-carboxylic acid

To a solution of methyl (3S,6S,10aR)-6-{[(tert-butoxy)carbonyl]amino}-9-methylidene-5-oxo-decahydropyrrolo[1,2-a]azocine-3-carboxylate (210 mg, 0.5958 mmol, 1 eq) in THF (6 mL) and water (2 mL) was added LiOH—H₂O (74.6 mg, 1.78 mmol, 3 eq). The reaction was stirred for 20 h at room temperature. The reaction was then concentrated under reduced pressure and purified by reverse phase chromatography [C₁₈ cartridge eluting with a gradient of 5-60% acetonitrile in water (with 0.1% FA)] to give (3S,6S,10aR)-6-{[(tert-butoxy)carbonyl]amino}-9-methylidene-5-oxo-decahydropyrrolo[1,2-a]azocine-3-carboxylic acid (186 mg, 0.5496 mmol, 92.5% yield) as a white solid. LCMS (ESI) m/z=339.2 [M+H]⁺.

### Step 3: Preparation of (3S,6S,10aR)-2,2-Dibromo-6-((tert-butoxycarbonyl)amino)-5-oxooctahydro-1H-spiro[cyclopropane-1,9-pyrrolo[1,2-a]azocine]-3-carboxylic acid

To a solution of (3S,6S,10aR)-6-{[(tert-butoxy)carbonyl]amino}-9-methylidene-5-oxo-decahydropyrrolo[1,2-a]azocine-3-carboxylic acid (186 mg, 0.5496 mmol, 1 eq) in CH₂Cl₂ (2 mL) was added bromoform (384 μL, 4.35 mmol, 7.9 eq) and TEBAC (15.0 mg, 65.9 μmol, 0.12 eq), followed by addition of a 50% wt aqueous solution of NaOH (0.56 mL, 7.0 mmol, 12.7 eq). The reaction was heated at reflux for 21 h. The reaction mixture was then diluted with 1 N aqueous NaOH and the product was extracted with CH₂Cl₂. The organic layer was concentrated under reduced pressure and dried under high vacuum. The resulting residue was purified by reverse phase chromatography [C18 cartridge using a gradient of 5-80% acetonitrile in water (with 0.1% FA)] to give (3S,6S,10aR)-2,2-dibromo-6-((tert-butoxycarbonyl)amino)-5-oxooctahydro-1H-spiro[cyclopropane-1,9-pyrrolo[1,2-a]azocine]-3-carboxylic acid (175 mg, 0.3429 mmol, 62.5% yield) as a white solid. LCMS (ESI) m/z=509.0 [M+H]⁺.

### Step 4: Preparation of (3S,6S,10aR)-6-((tert-Butoxycarbonyl)amino)-5-oxooctahydro-1H-spiro[cyclopropane-1,9-pyrrolo[1,2-a]azocine]-3-carboxylic acid

To a solution of (3S,6S,10aR)-2,2-dibromo-6-((tert-butoxycarbonyl)amino)-5-oxooctahydro-1H-spiro[cyclopropane-1,9-pyrrolo[1,2-a]azocine]-3-carboxylic acid (175 mg, 0.3429 mmol, 1 eq) in 2-propanol (6 mL) under N₂ (g) atmosphere was added KOH (115 mg, 2.05 mmol, 6 eq) and 10% Pd/C (50% wet, 170 mg, 0.1597 mmol, 0.466 eq). The reaction mixture was heated to 70° C. under H₂ (g) (40 psi) for 21 h. The reaction mixture was then diluted with MeOH and filtered over a pad of Celite®. The filtrate was concentrated under reduced pressure and subsequently purified by reverse phase chromatography [C₁₈ cartridge eluting with a gradient of 5-70% acetonitrile in water (with 0.1% FA)] to give (3S,6S,10aR)-6-((tert-butoxycarbonyl)amino)-5-oxooctahydro-1H-spiro[cyclopropane-1,9-pyrrolo[1,2-a]azocine]-3-carboxylic acid (104 mg, 0.2950 mmol, 86.6% yield) as a white solid. LCMS (ESI) m/z=353.2 [M+H]⁺.

The following intermediate in Table 16 was prepared according to the steps (Steps 1-4) outlined above for synthesis of (3S,6S,10aR)-6-((tert-butoxycarbonyl)amino)-5-oxooctahydro-1H-spiro[cyclopropane-1,9-pyrrolo[1,2-a]azocine]-3-carboxylic acid and starting from methyl (3S,6S,10aR)-6-{[(tert-butoxy)carbonyl]amino}-5,8-dioxo-decahydropyrrolo[1,2-a]azocine-3-carboxylate.

TABLE 16

| Name | Structure | LCMS |
|---|---|---|
| (3′S,6′S,10a′S)-6′-((tert- butoxycarbonyl)amino)-5′-oxooctahydro- 5′H-spiro[cyclopropane-1,8′-pyrrolo[1,2- a]azocine]-3′-carboxylic acid | 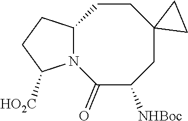 [SMILES: CC(C)(C)OC(=O)N[C@H]1CC2(CC[C@H]3CC[C@@H](C(=O)O)N3C1=O)CC2] | 382.4 [M + H]⁺ |

### Synthesis of (3S,6S,10S,10aR)-6-amino-10-methyl-5-oxodecahydropyrrolo[1,2-a]azocine-3-carboxylic acid

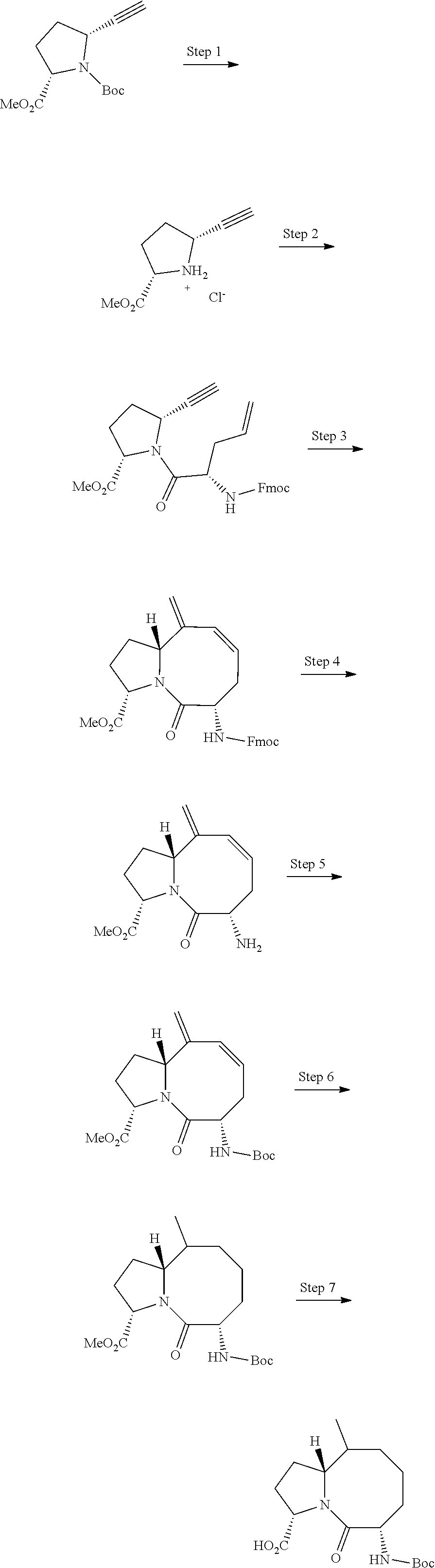

### Step 1: Preparation of Methyl (2S,5R)-5-ethynylpyrrolidine-2-carboxylate hydrochloride

To a solution of 1-(tert-butyl) 2-methyl (2S,5R)-5-ethynylpyrrolidine-1,2-dicarboxylate (52.0 g, 205 mmol, 1.00 eq) in EtOAc (260 mL) was added 4.0 M solution of HCl (154 mL, 3.00 eq) in EtOAc. After 3.5 h, the mixture was concentrated under reduced pressure to give methyl (2S,5R)-5-ethynylpyrrolidine-2-carboxylate hydrochloride (44.0 g, 232 mmol, HCl salt) as brown oil. ¹H NMR (400 MHz CDCl₃) δ 4.74-4.71 (m, 1H), 4.62 (t, J=7.2 Hz, 1H), 3.91 (s, 3H), 2.69 (d, J=1.6 Hz, 1H), 2.61-2.35 (m, 3H), 2.21-2.14 (m, 1H).

### Step 2: Preparation of Methyl (2S,5R)-1-((S)-2-((((9H-fluoren-9-yl)methoxy)carbonyl)amino)pent-4-enoyl)-5-ethynylpyrrolidine-2-carboxylate

To a mixture of methyl (2S,5R)-5-ethynylpyrrolidine-2-carboxylate hydrochloride (44.0 g, 232 mmol, 1.00 eq, HCl) and (S)-2-((((9H-fluoren-9-yl)methoxy)carbonyl)amino)pent-4-enoic acid (62.6 g, 186 mmol, 0.80 eq) in CH₂Cl₂ (880 mL) was added BOPCl (59.1 g, 232 mmol, 1.00 eq) and NaHCO₃(78.0 g, 928 mmol, 36.1 mL, 4.00 eq) under N₂ (g). The mixture was heated to 40° C. and stirred After 2 h, the mixture cooled to ambient temperatures and concentrated in vacuo. The residue was purified by column chromatography (petroleum ether/EtOAc=100/1 to 4/1) to give methyl (2S,5R)-1-((S)-2-((((9H-fluoren-9-yl)methoxy)carbonyl)amino)pent-4-enoyl)-5-ethynylpyrrolidine-2-carboxylate (85.0 g, 169 mmol, 93.9% purity) as yellow oil. LCMS (ESI) m/z=473.0 [M+H]⁺; ¹H NMR (400 MHz CDCl₃) δ 7.76 (d, J=7.6 Hz, 2H), 7.60-7.56 (m, 2H), 7.40 (t, J=7.6 Hz, 2H), 7.33-7.30 (m, 2H), 5.92-5.81 (m, 1H), 5.50 (d, J=8.4 Hz, 1H), 5.30-5.10 (m, 2H), 5.00-4.92 (m, 1H), 4.73-4.69 (m, 1H), 4.54-4.50 (m, 1H), 4.40-4.35 (m, 1H), 4.31-4.27 (m, 1H), 4.23-4.19 (m, 1H), 3.76-3.70 (m, 2H), 2.80-2.75 (m, 1H), 2.58-2.53 (m, 1H), 2.50-2.49 (m, 1H), 2.37-2.31 (m, 1H), 2.27-2.18 (m, 3H).

### Step 3: Preparation of Methyl (3S,6S,10aR,Z)-6-((((9H-fluoren-9-yl)methoxy)carbonyl)amino)-10-methylene-5-oxo-1,2,3,5,6,7,10,10a-octahydropyrrolo[1,2-a]azocine-3-carboxylate

A solution of methyl (2S,5R)-1-((S)-2-((((9H-fluoren-9-yl)methoxy)carbonyl)amino)pent-4-enoyl)-5-ethynylpyrrolidine-2-carboxylate in toluene (2.80 L) was sparged with ethylene for 10 min. To the solution was added Grubbs Generation I catalyst (3.65 g, 5.82 mmol, 0.05 eq) and the mixture was stirred under an atmosphere of ethylene. Three batches of equal scale were performed in parallel and combined for work-up. After 3 h, the reaction mixture was concentrated in vacuo. The resulting product was purified by reversed phase HPLC (0.10% formic acid condition) to afford methyl (3S,6S,10aR,Z)-6-((((9H-fluoren-9-yl)methoxy)carbonyl)amino)-10-methylene-5-oxo-1,2,3,5,6,7,10,10a-octahydropyrrolo[1,2-a]azocine-3-carboxylate (43.0 g, 87.1 mmol, 95.7% purity) as black brown oil. LCMS (ESI) m/z=473.0 [M+H]⁺; ¹H NMR (400 MHz CDCl₃) δ 7.77 (d, J=7.6 Hz, 2H), 7.62-7.58 (m, 2H), 7.41 (t, J=7.2 Hz, 2H), 7.33-7.29 (m, 2H), 6.24-6.15 (m, 1H), 5.94-5.81 (m, 1H), 5.58 (d, J=8.8 Hz, 1H), 5.27-5.16 (m, 1H), 5.04-5.01 (m, 1H), 4.81-4.69 (m, 1H), 0.4.54-4.50 (m, 1H), 4.40-4.20 (m, 3H), 3.77-3.70 (m, 3H), 2.79-2.66 (m, 1H), 2.57-2.49 (m, 1H), 2.38-2.16 (m, 3H), 2.11-2.07 (m, 1H).

### Step 4: Preparation of Methyl (3S,6S,10aR,Z)-6-amino-10-methylene-5-oxo-1,2,3,5,6,7,10,10a-octahydropyrrolo[1,2-a]azocine-3-carboxylate

To a solution of methyl (3S,6S,10aR,Z)-6-((((9H-fluoren-9-yl)methoxy)carbonyl)amino)-10-methylene-5-oxo-1,2,3,5,6,7,10,10a-octahydropyrrolo[1,2-a]azocine-3-carboxylate (13.0 g, 27.5 mmol, 1.00 eq) in THF (260 mL) was added piperidine (7.03 g, 82.5 mmol, 8.15 mL, 3.00 eq). After stirring for 2 h, the mixture was poured into a solution of aqueous 2 N HCl (200 mL) and the biphasic mixture was extracted with EtOAc (200 mL×2). The combined organic layers were discarded and the aqueous phase was basified with NaHCO₃ (adjusted to pH=8-9). The resulting basic aqueous layer was extracted with EtOAc (200 mL×2). The combined organic layers were discarded, the aqueous phase was collected to give a crude product. The crude product was purified by prep-HPLC (Phenomenex luna c18 250 mm*100 mm*10 um; mobile phase: [water (TFA)-acetonitrile]; B %: 10%-30%, 20 min) and lyophilizated to give methyl (3S,6S,10aR,Z)-6-amino-10-methylene-5-oxo-1,2,3,5,6,7,10,10a-octahydropyrrolo[1,2-a]azocine-3-carboxylate (2.65 g, 10.6 mmol, 38.5% yield) as a white solid. LCMS (ESI) m/z=250.9 [M+H]⁺; ¹H NMR (400 MHz CDCl₃) δ 8.30 (s, 3H), 6.30-6.23 (m, 1H), 5.89 (t, J=4.0 Hz, 1H), 5.29-5.25 (m, 1H), 5.04-5.01 (m, 1H), 4.88 (s, 1H), 4.63-4.61 (m, 1H), 4.46 (s, 1H), 3.61 (s, 3H), 0.2.49-2.43 (m, 1H), 2.39-2.03 (m, 1H), 1.93-1.92 (m, 1H), 1.91-1.82 (m, 1H).

### Step 5: Preparation of Methyl (3S,6S,10aR,Z)-6-((tert-butoxycarbonyl)amino)-10-methylene-5-oxo-1,2,3,5,6,7,10,10a-octahydropyrrolo[1,2-a]azocine-3-carboxylate

To a solution of methyl (3S,6S,10aR,Z)-6-amino-10-methylene-5-oxo-1,2,3,5,6,7,10,10a-octahydropyrrolo[1,2-a]azocine-3-carboxylate (2.55 g, 10.2 mmol, 1.00 eq) and Et₃N (2.06 g, 20.4 mmol, 2.84 mL, 2.00 eq) in CH₂Cl₂ (25.5 mL) was added Boc₂O (2.45 g, 11.2 mmol, 2.57 mL, 1.10 eq). The reaction was stirred for 2 h and subsequently poured into aqueous saturated NH₄Cl (50.0 mL). The biphasic mixture was extracted with EtOAc (30.0 mL×2). The combined organic layers were dried over Na₂SO₄ and concentrated to give a residue. The residue was purified by column chromatography (SiO₂, petroleum ether/EtOAc=100/1 to 10/1) to give methyl (3S,6S,10aR,Z)-6-((tert-butoxycarbonyl)amino)-10-methylene-5-oxo-1,2,3,5,6,7,10,10a-octahydropyrrolo[1,2-a]azocine-3-carboxylate (1.52 g, 4.26 mmol, 98.1% purity, 41.8% yield) as colorless oil. LCMS (ESI) m/z=251.2 [(M−100)+H]⁺; ¹H NMR (400 MHz CDCl₃) δ 6.21-6.14 (m, 1H), 5.90 (d, J=4.0 Hz, 1H), 5.76 (d, J=3.2 Hz, 1H), 5.20 (d, J=8.4 Hz, 1H), 4.99 (d, J=5.4 Hz, 1H), 4.79-4.72 (m, 3H), 4.65-4.61 (m, 1H), 3.71 (s, 3H), 0.2.61-2.59 (m, 1H), 2.53-2.38 (m, 2H), 2.07-2.04 (m, 2H), 1.43 (s, 9H).

### Step 6: Preparation of Methyl (3S,6S,10S,10aR)-6-amino-10-methyl-5-oxodecahydropyrrolo[1,2-a]azocine-3-carboxylate

To a solution (under N₂ (g)) of methyl (3S,6S,10aR,Z)-6-((tert-butoxycarbonyl)amino)-10-methylene-5-oxo-1,2,3,5,6,7,10,10a-octahydropyrrolo[1,2-a]azocine-3-carboxylate (1.52 g, 4.34 mmol, 1.00 eq) in EtOAc (15.2 mL) was carefully added 10% Pd/C (0.30 g, 868 umol, 0.20 eq) at 25° C. The suspension was subjected to three cycles of evacuation and purging with H₂ (g). The mixture was stirred under H₂ (g) (50 Psi) at 25° C. After stirring for 3 h, the reaction vessel was evacuated, and the reaction mixture was subjected to three cycles of evacuation and purging with N₂ (g). The reaction mixture was filtered over Celite® and the filter liquor was concentrated to give methyl (3S,6S,10S,10aR)-6-amino-10-methyl-5-oxodecahydropyrrolo[1,2-a]azocine-3-carboxylate (1.14 g, crude) as colorless oil. LCMS (ESI) m/z=355.0 [M+H]⁺.

### Step 7: Preparation of (3S,6S,10S,10aR)-6-amino-10-methyl-5-oxodecahydropyrrolo[1,2-a]azocine-3-carboxylic acid

To a solution of methyl (3S,6S,10S,10aR)-6-amino-10-methyl-5-oxodecahydropyrrolo[1,2-a]azocine-3-carboxylate (1.16 g, 3.26 mmol, 1.00 eq) in THF (11.0 mL) was added a solution of LiOH·H₂O (411 mg, 9.79 mmol, 3.00 eq) in H₂O (2.20 mL). After 12 h, H₂O (20.0 mL) was added to the reaction mixture and the solution was acidified with aqueous 1 N HCl to pH 2. The mixture was extracted with EtOAc (20.0 mL×3). The combined organic layers were dried over Na₂SO₄ and concentrated to give a residue. The residue was purified by prep-HPLC (column: Phenomenex luna C18 150*40 mm*15 um; mobile phase: [water (TFA)-ACN]; B %: 31%-61%, 10 min) and lyophilized to give (3S,6S,10S,10aR)-6-amino-10-methyl-5-oxodecahydropyrrolo[1,2-a]azocine-3-carboxylic acid (0.80 g, 2.35 mmol, 77.9% over two steps) as a white solid.

Two batches of equal scale were combined to yield (3S,6S,10S,10aR)-6-amino-10-methyl-5-oxodecahydropyrrolo[1,2-a]azocine-3-carboxylic acid (1.12 g, 4.66 mmol, 99.1% purity) as a white solid. LCMS (ESI) m/z=241.0 [(M−100)+H]⁺; ¹H NMR (400 MHz CDCl₃) δ 5.69 (d, J=3.0 Hz, 1H), 4.75-4.66 (m, 1H), 4.35-4.32 (m, 1H), 4.05 (t, J=8.8 Hz, 1H), 3.66-3.51 (m, 1H), 2.55-2.47 (m, 1H), 2.41-2.07 (m, 3H), 1.97-1.67 (m, 3H), 1.69-1.55 (m, 2H), 1.46 (s, 9H), 1.36-1.20 (m, 1H), 1.10-0.95 (m, 1H), 0.89-0.84 (m, 3H).

### Synthesis of (3S,6S,9R,10aR)-6-amino-9-(benzyloxy)-3-(3-(morpholine-4-carbonyl)azetidine-1-carbonyl)octahydropyrrolo[1,2-a]azocin-5(1H)-one

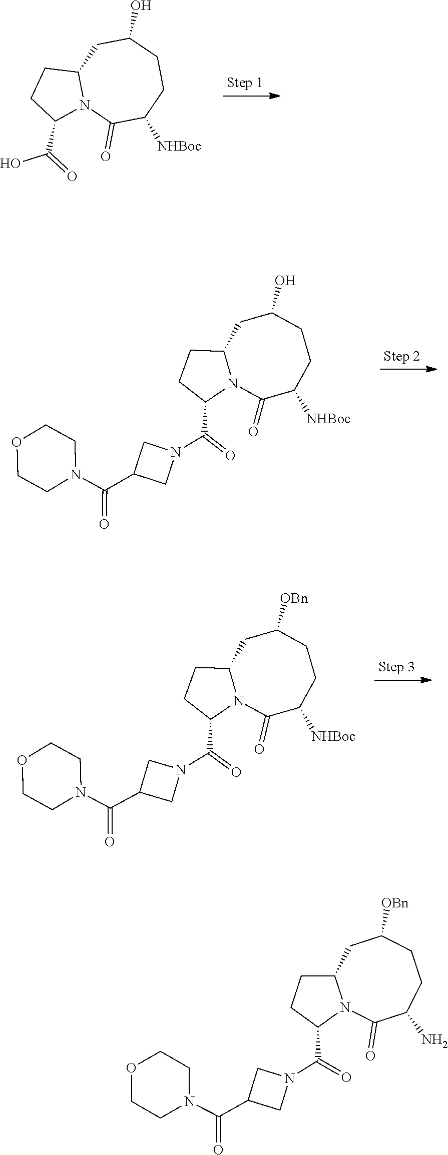 [SMILES: CC(C)(C)OC(=O)N[C@H]1CC[C@@H](O)C[C@H]2CC[C@@H](C(=O)O)N2C1=O ; CC(C)(C)OC(=O)N[C@H]1CC[C@@H](O)C[C@H]2CC[C@@H](C(=O)N3CC(C(=O)N4CCOCC4)C3)N2C1=O ; CC(C)(C)OC(=O)N[C@H]1CC[C@@H](OCc2ccccc2)C[C@H]2CC[C@@H](C(=O)N3CC(C(=O)N4CCOCC4)C3)N2C1=O ; N[C@H]1CC[C@@H](OCc2ccccc2)C[C@H]2CC[C@@H](C(=O)N3CC(C(=O)N4CCOCC4)C3)N2C1=O]

### Step 1: Preparation of tert-butyl N-[(3S,6S,9R,10aR)-9-hydroxy-3-[3-(morpholine-4-carbonyl)azetidine-1-carbonyl]-5-oxo-decahydropyrrolo[1,2-a]azocin-6-yl]carbamate

To a solution of (3S,6S,9R,10aR)-6-{[(tert-butoxy)carbonyl]amino}-9-hydroxy-5-oxodecahydropyrrolo[1,2-a]azocine-3-carboxylic acid (0.780 g, 2.27 mmol, 1 eq), 4-(azetidine-3-carbonyl)morpholine trifluoroacetic salt (645 mg, 2.27 mmol, 1 eq) and N,N-diisopropylethylamine (2.35 mL, 13.6 mmol, 6 eq) in DMF (10 mL) was added HATU (1.03 g, 2.72 mmol, 1.2 eq). The reaction was stirred at room temperature 2 h. The reaction mixture was then concentrated in vacuo and subsequently purified by reverse phase chromatography [C18 cartridge eluting with a gradient of 5-75% acetonitrile in water (with 0.1% FA)] to give tert-butyl N-[(3S,6S,9R,10aR)-9-hydroxy-3-[3-(morpholine-4-carbonyl)azetidine-1-carbonyl]-5-oxo-decahydropyrrolo[1,2-a]azocin-6-yl]carbamate (1.16 g, 2.34 mmol, 103% yield) as a beige foam. LCMS (ESI) m/z=495.3 [M+H]⁺.

### Step 2: Preparation of Tert-Butyl ((3S,6S,9R,10aR)-9-(benzyloxy)-3-(3-(morpholine-4-carbonyl)azetidine-1-carbonyl)-5-oxodecahydropyrrolo[1,2-a]azocin-6-yl)carbamate

To a solution of tert-butyl N-[(3S,6S,9R,10aR)-9-hydroxy-3-[3-(morpholine-4-carbonyl)azetidine-1-carbonyl]-5-oxo-decahydropyrrolo[1,2-a]azocin-6-yl]carbamate (75 mg, 0.1516 mmol, 1 eq) in CH₂Cl₂ (3 mL) was added Et₃N (63.2 μL, 454 μmol, 3 eq) and TMSCl (28.7 μL, 227 μmol, 1.5 eq) and the mixture was stirred at room temperature. After 30 min, brine (10 mL) was added, and the organic materials were extracted with CH₂Cl₂ (10 mL×2). The combined organic extracts were dried with sodium sulfate, filtered and concentrated in vacuo to give crude tert-butyl N-[(3S,6S,9R,10aR)-3-[3-(morpholine-4-carbonyl)azetidine-1-carbonyl]-5-oxo-9-[(trimethylsilyl)oxy]-decahydropyrrolo[1,2-a]azocin-6-yl]carbamate (85.9 mg, 0.1516 mmol, 100% yield) as a white solid.

A solution of tert-butyl N-[(3S,6S,9R,10aR)-3-[3-(morpholine-4-carbonyl)azetidine-1-carbonyl]-5-oxo-9-[(trimethylsilyl)oxy]-decahydropyrrolo[1,2-a]azocin-6-yl]carbamate (85.9 mg, 0.1516 mmol, 1 eq) in CH₂Cl₂ (5 mL) was cooled to −78° C., followed by addition of benzaldehyde (22.9 μL, 227 μmol, 1.5 eq), Et₃SiH (36.1 μL, 227 μmol, 1.5 eq) and trimethylsilyl triflate (20.4 μL, 113 μmol, 0.75 eq). The reaction was stirred for 10 min at −78° C. and then stirred for 90 min at 0° C. The reaction was quenched by the addition of a saturated aqueous solution of NaHCO₃ (10 mL) and the product was extracted with in CH₂Cl₂ (10 mL×3). The combined organic extracts were dried with sodium sulfate and filtered to give a solution of tert-butyl ((3S,6S,9R,10aR)-9-(benzyloxy)-3-(3-(morpholine-4-carbonyl)azetidine-1-carbonyl)-5-oxodecahydropyrrolo[1,2-a]azocin-6-yl)carbamate

### Step 3: Preparation of (3S,6S,9R,10aR)-6-amino-9-(benzyloxy)-3-[3-(morpholine-4-carbonyl)azetidine-1-carbonyl]-decahydropyrrolo[1,2-a]azocin-5-one

To the solution was added trifluoroacetic acid (0.5 mL) and the reaction was stirred for 30 min at room temperature then concentrated under reduced pressure. The reaction was purified by reverse phase chromatography [C18 cartridge eluting with a gradient of 5-40% Acetonitrile in water (with 0.1% FA)] to give (3S,6S,9R,10aR)-6-amino-9-(benzyloxy)-3-[3-(morpholine-4-carbonyl)azetidine-1-carbonyl]-decahydropyrrolo[1,2-a]azocin-5-one (41.0 mg, 0.08460 mmol, 56.0% yield) as a white solid. LCMS (ESI) m/z 485.4 [M+H]⁺.

The following intermediates in Table 17 were prepared according to the protocol outlined for synthesis of (3S,6S,9R,10aR)-6-amino-9-(benzyloxy)-3-[3-(morpholine-4-carbonyl)azetidine-1-carbonyl]-decahydropyrrolo[1,2-a]azocin-5-one and using the appropriate advanced intermediate(s) as starting materials. Where appropriate the final treatment with TFA (Step 3) was omitted and N-Boc protected amines were isolated.

TABLE 17

| Name | Structure | LCMS |
|---|---|---|
| tert-butyl ((3S,6S,8S,10aR)-8- (benzyloxy)-3-(3-(morpholine-4- carbonyl)azetidine-1-carbonyl)-5- oxodecahydropyrrolo[1,2-a]azocin-6- yl)carbamate | 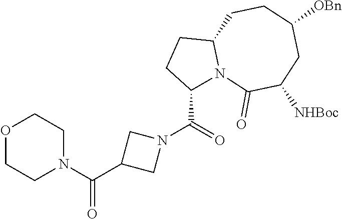 [SMILES: CC(C)(C)OC(=O)N[C@H]1C[C@@H](OCc2ccccc2)CC[C@H]2CC[C@@H](C(=O)N3CC(C(=O)N4CCOCC4)C3)N2C1=O] | 485.4 [M + H]⁺ |
| tert-butyl ((3S,6S,8R,10aR)-8- (benzyloxy)-3-(3-(morpholine-4- carbonyl)azetidine-1-carbonyl)-5- oxodecahydropyrrolo[1,2-a]azocin-6- yl)carbamate | 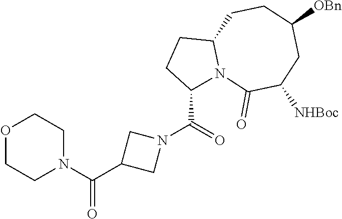 [SMILES: CC(C)(C)OC(=O)N[C@H]1C[C@H](OCc2ccccc2)CC[C@H]2CC[C@@H](C(=O)N3CC(C(=O)N4CCOCC4)C3)N2C1=O] | 485.4 [M + H]⁺ |
| tert-butyl ((3S,6S,9R,10aR)-9-ethoxy- 3-(3-(morpholine-4- carbonyl)azetidine-1-carbonyl)-5- oxodecahydropyrrolo[1,2-a]azocin-6- yl)carbamate | 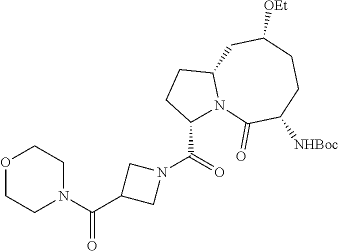 [SMILES: CCO[C@@H]1CC[C@H](NC(=O)OC(C)(C)C)C(=O)N2[C@H](CC[C@H]2C(=O)N2CC(C(=O)N3CCOCC3)C2)C1] | 523.3 [M + H]⁺ |
| tert-butyl ((3S,6S,8S,10aR)-8-ethoxy- 3-(3-(morpholine-4- carbonyl)azetidine-1-carbonyl)-5- oxodecahydropyrrolo[1,2-a]azocin-6- yl)carbamate | 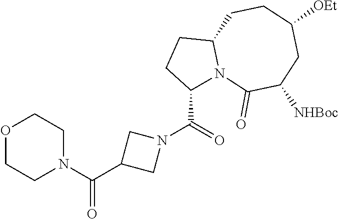 [SMILES: CCO[C@H]1CC[C@H]2CC[C@@H](C(=O)N3CC(C(=O)N4CCOCC4)C3)N2C(=O)[C@@H](NC(=O)OC(C)(C)C)C1] | 523.6 [M + H]⁺ |
| tert-butyl ((3S,6S,8R,10aR)-8-ethoxy- 3-(3-(morpholine-4- carbonyl)azetidine-1-carbonyl)-5- oxodecahydropyrrolo[1,2-a]azocin-6- yl)carbamate | 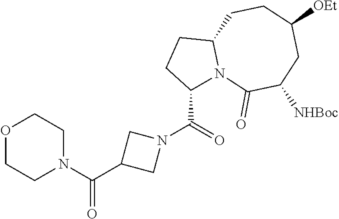 [SMILES: CCO[C@@H]1CC[C@H]2CC[C@@H](C(=O)N3CC(C(=O)N4CCOCC4)C3)N2C(=O)[C@@H](NC(=O)OC(C)(C)C)C1] | 523.6 [M + H]⁺ |

### Synthesis of tert-butyl ((3R,6R,8R,9R,10aS)-8,9-dihydroxy-5-oxo-3-(3-phenylpyrrolidine-1-carbonyl)decahydropyrrolo[1,2-a]azocin-6-yl)carbamate, tert-butyl ((3S,6S,8R,9R,10aR)-9-hydroxy-8-methyl-5-oxo-3-((S)-3-phenylpyrrolidine-1-carbonyl)decahydropyrrolo[1,2-a]azocin-6-yl)carbamate, and tert-butyl ((3S,6S,8S,9S,10aR)-8-hydroxy-9-methyl-5-oxo-3-((S)-3-phenylpyrrolidine-1-carbonyl)decahydropyrrolo[1,2-a]azocin-6-yl)carbamate

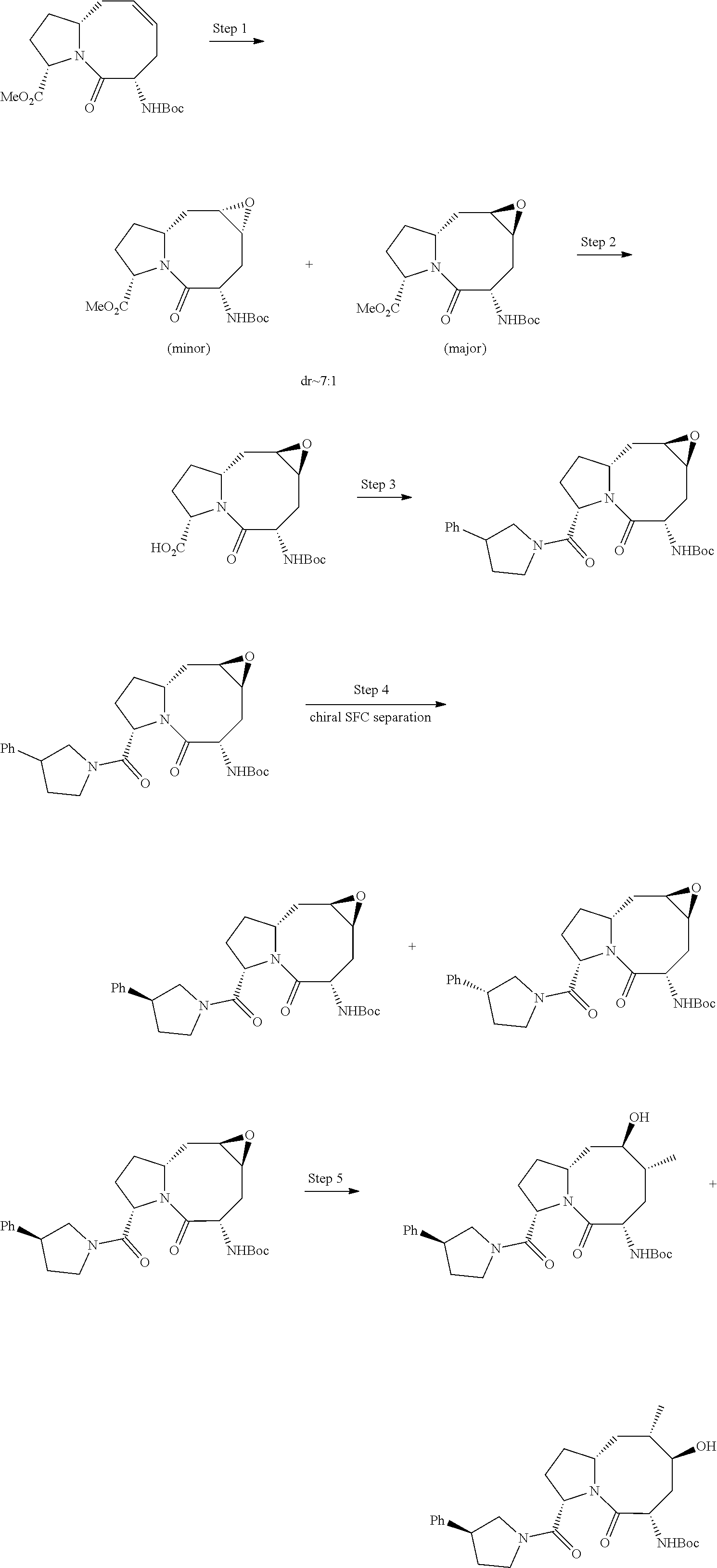

### Step 1: Preparation of Methyl (1aS,3S,6S,8aR,9aR)-3-((tert-butoxycarbonyl)amino)-4-oxodecahydrooxireno[2,3-d]pyrrolo[1,2-a]azocine-6-carboxylate and methyl (1aR,3S,6S,8aR,9aS)-3-((tert-butoxycarbonyl)amino)-4-oxodecahydrooxireno[2,3-d]pyrrolo[1,2-a]azocine-6-carboxylate

To a cooled (0° C.) solution of 1H-pyrazole (40.2 mg, 591 μmol, 0.1 eq), methyltrioxorhenium (14.7 mg, 59.1 μmol, 0.01 eq) and H₂O₂(6.66 g, 59.1 mmol, 10 eq) in CF₃CH₂OH (3 mL) was slowly added methyl (3S,6S,10aR,Z)-6-((tert-butoxycarbonyl)amino)-5-oxo-1,2,3,5,6,7,10,10a-octahydropyrrolo[1,2-a]azocine-3-carboxylate (2.00 g, 5.91 mmol, 1 eq). The reaction mixture was warmed to room temperature and stirred for 4 h. To the mixture was added a solution of saturated aqueous NaHSO₃ and the mixture was extracted with CH₂Cl₂ (20 mL×3). The combined organic layers were washed with brine (20 mL), dried over with anhydrous Na₂SO₄, and concentrated under reduced pressure. The resulting residue was purified by C18 column (gradient 15% to 60% acetonitrile in water) to give methyl (1aS,3S,6S,8aR,9aR)-3-((tert-butoxycarbonyl)amino)-4-oxodecahydrooxireno[2,3-d]pyrrolo[1,2-a]azocine-6-carboxylate (1.50 g, 4.23 mmol, 72% yield) as the major product major adduct and methyl (1aR,3S,6S,8aR,9aS)-3-((tert-butoxycarbonyl)amino)-4-oxodecahydrooxireno[2,3-d]pyrrolo[1,2-a]azocine-6-carboxylate (minor adduct) (190 mg, 0.54 mmol, 9% yield) as the minor adduct

Methyl (1aS,3S,6S,8aR,9aR)-3-((tert-butoxycarbonyl)amino)-4-oxodecahydrooxireno[2,3-d]pyrrolo[1,2-a]azocine-6-carboxylate (major diastereomer): LCMS (ESI) m/z=355 [M+H]⁺; ¹H NMR (400 MHz, CDCl₃) δ 5.51 (d, J=6.9 Hz, 1H), 4.68 (dd, J=14.9, 7.6 Hz, 1H), 4.60-4.48 (m, 1H), 4.27-4.13 (m, 1H), 3.74 (s, 3H), 3.29-3.20 (m, 1H), 3.18-3.08 (m, 1H), 2.55-2.36 (m, 2H), 2.34-2.13 (m, 3H), 2.09-1.91 (m, 3H), 1.42 (s, 9H).

Methyl (1aR,3S,6S,8aR,9aS)-3-((tert-butoxycarbonyl)amino)-4-oxodecahydrooxireno[2,3-d]pyrrolo[1,2-a]azocine-6-carboxylate (minor diastereomer): LCMS (ESI) m/z=355 [M+H]⁺; ¹H NMR (400 MHz, CDCl₃) δ 5.90 (d, J=6.4 Hz, 1H), 4.53-4.42 (m, 2H), 4.14 (t, J=6.3 Hz, 1H), 3.75 (s, 3H), 3.31-3.19 (m, 1H), 3.11-3.06 (m, 1H), 2.37-2.27 (m, 2H), 2.25-2.11 (m, 3H), 2.03-1.87 (m, 3H), 1.44 (s, 9H).

### Step 2: Preparation of (1aS,3S,6S,8aR,9aR)-3-((tert-butoxycarbonyl)amino)-4-oxodecahydrooxireno[2,3-d]pyrrolo[1,2-a]azocine-6-carboxylic acid

To a solution of methyl (1aS,3S,6S,8aR,9aR)-3-((tert-butoxycarbonyl)amino)-4-oxodecahydrooxireno[2,3-d]pyrrolo[1,2-a]azocine-6-carboxylate (390 mg, 1.10 mmol, 1.0 eq) in a mixture of THF (8 mL) and H₂O (2 mL) was added LiOH—H₂O (138 mg, 3.30 mmol, 3.0 eq). The reaction the mixture was stirred for 4 h at room temperature and subsequently acidified with an aqueous solution of 1 N HCl to pH=5-6. The resulting acidic mixture was extracted with EtOAc (20 mL×3). The combined organic layers were washed with brine (20 mL), dried over with anhydrous Na₂SO₄, and concentrated under reduced pressure. The crude product was purified on C18 column (gradient 15% to 60% acetonitrile in water) to give (1aS,3S,6S,8aR,9aR)-3-((tert-butoxycarbonyl)amino)-4-oxodecahydrooxireno[2,3-d]pyrrolo[1,2-a]azocine-6-carboxylic acid (300 mg, 0.88 mmol, 80% yield) as white solids. LCMS (ESI) m/z=341 [M+H]⁺.

### Step 3: Preparation of Tert-Butyl ((1aS,3S,6S,8aR,9aR)-4-oxo-6-(3-phenylpyrrolidine-1-carbonyl)decahydrooxireno[2,3-d]pyrrolo[1,2-a]azocin-3-yl)carbamate

To a solution of (1aS,3S,6S,8aR,9aR)-3-((tert-butoxycarbonyl)amino)-4-oxodecahydrooxireno[2,3-d]pyrrolo[1,2-a]azocine-6-carboxylic acid (300 mg, 0.88 mmol, 1 eq), Et₃N (445 mg, 4.40 mmol, 5 eq) and rac-3-phenylpyrrolidine (129 mg, 0.88 mmol, 1 eq) in DMF (10 mL) was added HATU (399 mg, 1.05 mmol, 1.2 eq) in one portion. The resulting mixture was stirred at room temperature for additional 12 h and subsequently diluted with water (5 mL). The biphasic mixture was extracted with CH₂Cl₂ (10 mL×3). The combined organic layers and washed with brine (20 mL), dried over with anhydrous Na₂SO₄, and concentrated under reduced pressure. The resulting residue was purified by flash column chromatography on silica gel to afford tert-butyl ((1aS,3S,6S,8aR,9aR)-4-oxo-6-(3-phenylpyrrolidine-1-carbonyl)decahydrooxireno[2,3-d]pyrrolo[1,2-a]azocin-3-yl)carbamate (200 mg, 0.43 mmol, 48% yield) as clear oil. LCMS (ESI) m/z=470.3 [M+H]⁺; ¹H NMR (400 MHz, CDCl₃) δ 7.37-7.30 (m, 2H), 7.27-7.19 (m, 3H), 5.87-5.37 (m, 1H), 4.74-4.41 (m, 2H), 4.36-4.08 (m, 2H), 4.02-3.65 (m, 2H), 3.61-3.31 (m, 4H), 2.50-2.26 (m, 3H), 2.25-1.92 (m, 6H), 1.90-1.82 (m, 1H), 1.43 (s, 9H).

### Step 4: Preparation of Tert-Butyl ((1aS,3S,6S,8aR,9aR)-4-oxo-6-((S)-3-phenylpyrrolidine-1-carbonyl)decahydrooxireno[2,3-d]pyrrolo[1,2-a]azocin-3-yl)carbamate and tert-butyl ((1aS,3S,6S,8aR,9aR)-4-oxo-6-((R)-3-phenylpyrrolidine-1-carbonyl)decahydrooxireno[2,3-d]pyrrolo[1,2-a]azocin-3-yl)carbamate

Rac-tert-butyl ((1aR,3R,6R,8aS,9aS)-4-oxo-6-(3-phenylpyrrolidine-1-carbonyl)decahydrooxireno[2,3-d]pyrrolo[1,2-a]azocin-3-yl)carbamate (600 mg, 1.27 mmol, 1 eq), triethylamine (976 mg, 9.65 mmol, 5 eq) and (3S)-3-phenylpyrrolidine (284 mg, 1.93 mmol, 1 eq) was separated by SFC (ChiralCel OX, 250×21.2 mm I.D., 5 μm, A for CO₂ and B for MeOH+0.1% NH₄OH, 40 mL/min) to yield Peak 1: tert-butyl ((1aS,3S,6S,8aR,9aR)-4-oxo-6-((R)- or (S)-3-phenylpyrrolidine-1-carbonyl)decahydrooxireno[2,3-d]pyrrolo[1,2-a]azocin-3-yl)carbamate (400 mg, 0.8518 mmol, 67.1% yield). LCMS (ESI) m/z=470.3 [M+H]⁺ and Peak 2: tert-butyl ((1aS,3S,6S,8aR,9aR)-4-oxo-6-((S)- or (R)-3-phenylpyrrolidine-1-carbonyl)decahydrooxireno[2,3-d]pyrrolo[1,2-a]azocin-3-yl)carbamate (130 mg, 0.2768 mmol, 21.8% yield) as clear oil. LCMS (ESI) m/z=470.3 [M+H]⁺.

### Step 5: Preparation of Tert-Butyl ((3S,6S,8R,9R,10aR)-9-hydroxy-8-methyl-5-oxo-3-((S)-3-phenylpyrrolidine-1-carbonyl)decahydropyrrolo[1,2-a]azocin-6-yl)carbamate and tert-butyl ((3S,6S,8S,9S,10aR)-8-hydroxy-9-methyl-5-oxo-3-((S)-3-phenylpyrrolidine-1-carbonyl)decahydropyrrolo[1,2-a]azocin-6-yl)carbamate

To a cooled (0° C.) solution of tert-butyl ((1aS,3S,6S,8aR,9aR)-4-oxo-6-((R)- or (S)-3-phenylpyrrolidine-1-carbonyl)decahydrooxireno[2,3-d]pyrrolo[1,2-a]azocin-3-yl)carbamate (Peak 1) (200 mg, 0.43 mmol, 1 eq) in toluene (5 mL) was slowly added AlMe₃ (613 mg, 8.51 mmol, 20 eq) in a dropwise manner. The reaction mixture was subsequently warmed to room temperature. After 30 min, the reaction mixture was cooled to 0° C. and MeOH (10 mL) was subsequently added to quench excess AlMe₃. The resulting mixture was concentrated under reduced pressure and purified on C18 column (gradient 20% to 70% acetonitrile in water) to give an inseparable regioisomeric mixtures of epoxide ring opened products (160 mg, 0.33 mmol, 77% yield). LCMS (ESI) m/z=486.3 [M+H]⁺; ¹H NMR (400 MHz, mixture of isomers, CDCl₃) δ 7.40-7.28 (m, 3H), 7.26-7.21 (m, 2H), 5.78-5.50 (m, 1H), 4.73-4.49 (m, 2H), 4.36-4.09 (m, 2H), 4.04-3.30 (m, 6H), 2.44-1.97 (m, 10H), 1.43 (s, 9H), 1.13-0.83 (m, 3H).

The mixture of regioisomeric ring opened products (60 mg, 0.124 mmol) was purified by SFC (ChiralPak R,R-WHELK, 250×21.2 mm I.D., 5 μm; Mobile phase: A for CO₂ and B for MeOH+0.1% NH₄OH Gradient: B 25%; flow rate: 40 mL/min) to yield:

Peak 1: tert-butyl ((3S,6S,8R,9R,10aR)-9-hydroxy-8-methyl-5-oxo-3-((R)- or (S)-3-phenylpyrrolidine-1-carbonyl)decahydropyrrolo[1,2-a]azocin-6-yl)carbamate (3.00 mg, 0.006177 mmol). LCMS (ESI) m/z=486.3 [M+H]⁺.

Peak 2: tert-butyl ((3S,6S,8S,9S,10aR)-8-hydroxy-9-methyl-5-oxo-3-((S)- or (R)-3-phenylpyrrolidine-1-carbonyl)decahydropyrrolo[1,2-a]azocin-6-yl)carbamate (5.00 mg, 0.01029 mmol) as white solids. LCMS (ESI) m/z=486.3 [M+H]⁺.

### Procedures for Syntheses of Linkers

The following intermediates in Table 18 were prepared according to the protocol described in WO 2020/205467.

TABLE 18

| Name | Structure |
|---|---|
| 5- ((diethoxyphosphoryl)difluoromethyl) benzo[b]thiophene-2-carboxylic acid | 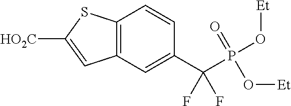 [SMILES: CCOP(=O)(OCC)C(F)(F)c1ccc2sc(C(=O)O)cc2c1] |
| 7-((diethoxyphosphoryl)difluoromethyl)-2- naphthoic acid | 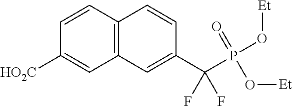 [SMILES: CCOP(=O)(OCC)C(F)(F)c1ccc2ccc(C(=O)O)cc2c1] |
| 5-((diethoxyphosphoryl)difluoromethyl)-1H- indole-2-carboxylic acid | 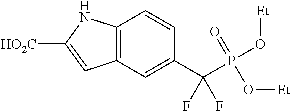 [SMILES: CCOP(=O)(OCC)C(F)(F)c1ccc2[nH]c(C(=O)O)cc2c1] |

### Synthesis of (E)-3-(4-((diethoxyphosphoryl)difluoromethyl)phenyl)acrylic acid

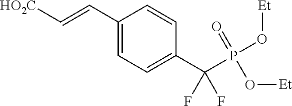 [SMILES: CCOP(=O)(OCC)C(F)(F)c1ccc(/C=C/C(=O)O)cc1]

(E)-3-(4-((diethoxyphosphoryl)difluoromethyl)phenyl)acrylic acid was prepared according to protocol described in US 2004/0225146.

### Representative Procedure for Preparation of Activated Ester Phosphonic Acids

### Synthesis of (difluoro(2-((4-nitrophenoxy)carbonyl)benzo[b]thiophen-5-yl)methyl)phosphonic acid

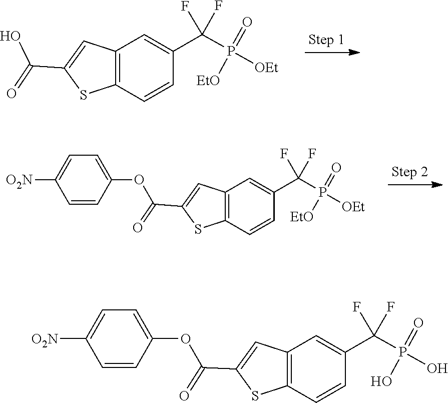 [SMILES: CCOP(=O)(OCC)C(F)(F)c1ccc2sc(C(=O)O)cc2c1 ; CCOP(=O)(OCC)C(F)(F)c1ccc2sc(C(=O)Oc3ccc([N+](=O)[O-])cc3)cc2c1 ; O=C(Oc1ccc([N+](=O)[O-])cc1)c1cc2cc(C(F)(F)P(=O)(O)O)ccc2s1]

### Step 1: Preparation of 4-nitrophenyl 5-((diethoxyphosphoryl)difluoromethyl)benzo[b]thiophene-2-carboxylate

To a mixture of 5-[(diethoxyphosphoryl)difluoromethyl]-1-benzothiophene-2-carboxylic acid (10.0 g, 27.4 mmol), EDCI (7.85 g, 41.0 mmol) and DMAP (836 mg, 6.85 mmol) in CH₂Cl₂ (80 mL) was stirred at room temperature. After 15 min, 4-nitrophenol (4.75 g, 34.2 mmol) was added and the resulting yellow mixture was stirred at room temperature for 18 h. The reaction was quenched with water (30 mL) and the product was extracted with CH₂Cl₂ (10 mL×2). The combined organic extracts were washed with brine, dried with sodium sulfate, filtered and concentrated in vacuo. The crude residue was purified by reverse phase chromatography [C18 cartridge eluting with a gradient of 5-100% acetonitrile in water] and the appropriate fractions were concentrated to give 4-nitrophenyl 5-((diethoxyphosphoryl)difluoromethyl)benzo[b]thiophene-2-carboxylate (7.80 g, 16.0 mmol, 59.0% yield) as a yellow solid. LCMS m/z=486.2 [M+H]⁺; ¹H NMR (400 MHz, DMSO-d₆) δ 8.32-8.37 (m, 3H), 8.22 (s, 1H), 8.01 (d, J=9.1 Hz, 1H), 7.77 (d, J=7.8 Hz, 1H), 7.51-7.45 (m, 2H), 4.14-4.32 (m, 4H), 1.34 (t, J=7.8 Hz, 6H).

### Step 2: Preparation of (difluoro(2-((4-nitrophenoxy)carbonyl)benzo[b]thiophen-5-yl)methyl)phosphonic acid

To a cooled (0° C.) solution of 4-nitrophenyl 5-((diethoxyphosphoryl)difluoromethyl)benzo[b]thiophene-2-carboxylate (4.47 g, 9.20 mmol) in CH₂Cl₂ (39 mL) was added N,O-bis(trimethylsilyl)trifluoroacetamide (12.1 mL, 46.0 mmol) and iodotrimethylsilane (5.23 mL, 36.8 mmol) as a solution in CH₂Cl₂ (10 mL). The reaction mixture was gradually allowed to warm to ambient temperatures. To the reaction mixture was added a mixture of 2:1 H₂O/acetonitrile (with 0.1% TFA) (50 mL) and precipitation of product was observed. The volatiles were removed in vacuo and the crude residue was suspended in a mixture of acetonitrile/water solution (1:1 v/v, 100 mL). The suspension was filtered, the solids were washed with a 2:1 mixture acetonitrile/water solution, and the solid were dried under reduced pressure to afford [difluoro({2-[(4-nitrophenoxy)carbonyl]-1-benzothiophen-5-yl})methyl]phosphonic acid (6.5 g, 94%) as a beige solid. The filtrate was concentrated to 50% of solvent volume and the resulting suspension was filtered and washed with 1:2 acetonitrile/water solution. The solid was dried under reduced pressure to afford additional [difluoro({2-[(4-nitrophenoxy)carbonyl]-1-benzothiophen-5-yl})methyl]phosphonic acid (0.4 g) as a beige solid. Both products were lyophilized to give (difluoro(2-((4-nitrophenoxy)carbonyl)benzo[b]thiophen-5-yl)methyl)phosphonic acid (6.90 g, 16.0 mmol, 98.0% yield). ¹H NMR (400 MHz, DMSO-d₆) δ 7.66-7.74 (m, 3H), 8.27 (d, J=8.3 Hz, 1H), 8.30 (s, 1H), 8.36-8.41 (m, 2H), 8.66 (s, 1H).

The following intermediates in Table 19 were prepared using a similar protocol outlined above for synthesis of [difluoro({2-[(4-nitrophenoxy)carbonyl]-1-benzothiophen-5-yl})methyl]phosphonic acid and utilizing the appropriate advanced intermediate(s) as starting material(s).

TABLE 19

| Name | Structure | LC-MS |
|---|---|---|
| (difluoro(7-((4- nitrophenoxy)carbonyl) naphthalen-2- yl)methyl)phosphonic acid | 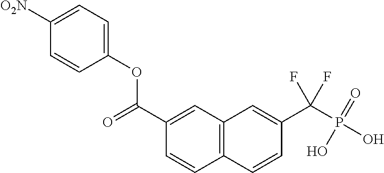 [SMILES: O=C(Oc1ccc([N+](=O)[O-])cc1)c1ccc2ccc(C(F)(F)P(=O)(O)O)cc2c1] | 424.1 [M + H]⁺ |
| (difluoro(2- ((perfluorophenoxy) carbonyl)benzo[b] thiophen-5-yl)methyl) phosphonic acid | 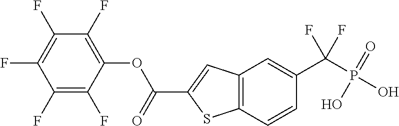 [SMILES: O=C(Oc1c(F)c(F)c(F)c(F)c1F)c1cc2cc(C(F)(F)P(=O)(O)O)ccc2s1] | 475.0 [M + H]⁺ |
| (difluoro(7- ((perfluorophenoxy) carbonyl)naphthalen- 2-yl)methyl)phosphonic acid | 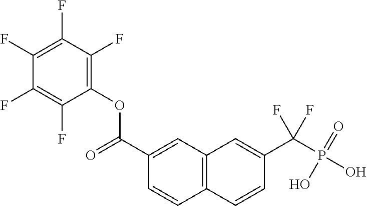 [SMILES: O=C(Oc1c(F)c(F)c(F)c(F)c1F)c1ccc2ccc(C(F)(F)P(=O)(O)O)cc2c1] | 467.0 [M − H]⁻ |

### Synthesis of ((2-((perfluorophenoxy)carbonyl)benzo[b]thiophen-5-yl)methyl)phosphonic acid

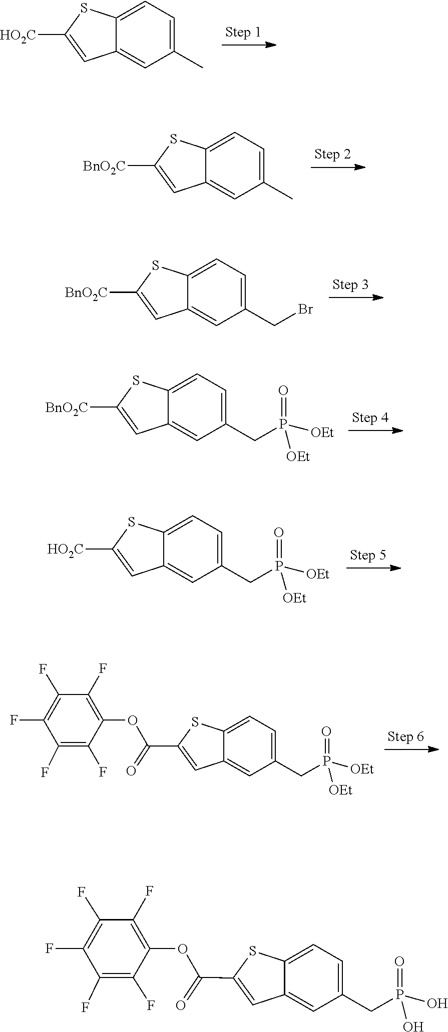

### Preparation of 5-methylbenzo[b]thiophene-2-carboxylic acid

5-Methylbenzo[b]thiophene-2-carboxylic acid was prepared according to the procedure described in WO 2016/100184.

### Step 1: Preparation of Benzyl 5-methylbenzo[b]thiophene-2-carboxylate

To a solution of 5-methylbenzo[b]thiophene-2-carboxylic acid (21.2 g, 110.0 mmol, 1.0 eq) and K₂CO₃ (30.4 g, 220.0 mmol, 2.0 eq) in DMF (200 mL) was added benzyl bromide (20.6 g, 121.0 mmol, 1.1 eq). The mixture was stirred at room temperature for 14 h. The reaction mixture was poured into ice water (400 mL) and stirred for 5 min. The resulting solids were filtered, and the filter cake was washed with water (50 mL), dried in vacuum to give benzyl 5-methylbenzo[b]thiophene-2-carboxylate (30.1 g, 106.0 mmol, 97% yield) as a yellow solid. ¹H NMR (400 MHz, CDCl₃) δ 8.01 (s, 1H), 7.71 (t, J=12.2 Hz, 1H), 7.65 (s, 1H), 7.46 (d, J=6.8 Hz, 2H), 7.42-7.35 (m, 3H), 7.29-7.26 (m, 1H), 5.38 (s, 2H), 2.47 (s, 3H).

### Step 2: Preparation of Benzyl 5-(bromomethyl)benzo[b]thiophene-2-carboxylate

To a solution of benzyl 5-methylbenzo[b]thiophene-2-carboxylate (15.0 g, 53.1 mmol, 1.0 eq) and NBS (10.3 g, 58.4 mmol, 1.1 eq) in CCl₄ (30 mL) was added benzoyl peroxide (1.3 g, 5.31 mmol, 0.1 eq). The reaction flask was subjected to three cycles of evacuation and backfilling with N₂ (g). The mixture was stirred at 80° C. for 16 h under constant atmosphere of N₂ (g). The reaction mixture was concentrated under reduced pressure. The residue was purified by flash column chromatography on silica gel to give benzyl 5-(bromomethyl)benzo[b]thiophene-2-carboxylate (6.80 g, 18.8 mmol, 36% yield) as a yellow solid.

### Step 3: Preparation of Benzyl 5-((diethoxyphosphoryl)methyl)benzo[b]thiophene-2-carboxylate

A solution of benzyl 5-(bromomethyl)benzo[b]thiophene-2-carboxylate (10.3 g, 28.5 mmol, 1.0 eq) dissolved in triethyl phosphite (30.0 g, 180.0 mmol, 6.3 eq) was stirred at 100° C. for 5 h. The reaction mixture was concentrated under reduced pressure directly, the residue was purified by flash column chromatography on silica gel to give benzyl 5-((diethoxyphosphoryl)methyl)benzo[b]thiophene-2-carboxylate (6.5 g, 15.5 mmol, 55% yield) as a colorless oil. LCMS (ESI) m/z=419 [M+H]⁺; ¹H NMR (400 MHz, CDCl₃) δ 8.04 (s, 1H), 7.80 (d, J=8.3 Hz, 2H), 7.49-7.33 (m, 6H), 5.39 (s, 2H), 4.08-3.93 (m, 4H), 3.26 (d, J=21.5 Hz, 2H), 1.24 (t, J=7.1 Hz, 6H).

### Step 4: Preparation of 5-((diethoxyphosphoryl)methyl)benzo[b]thiophene-2-carboxylic acid

To a solution of benzyl 5-((diethoxyphosphoryl)methyl)benzo[b]thiophene-2-carboxylate (5.6 g, 13.3 mmol, 1.0 eq) in dissolved in a mixture of THF (80 mL) and H₂O (10 mL) was added LiOH (1.10 g, 26.6 mmol, 2.0 eq). The mixture was stirred at room temperature for 3 h and subsequently acidified with aqueous solution of 1 N HCl (adjusted to pH ˜3-4). The product precipitated out of solution upon acidification. The resulting solids were filtered, the filter cake was washed with water (20 mL×2), and the solids were dried under vacuum to give 5-((diethoxyphosphoryl)methyl)benzo[b]thiophene-2-carboxylic acid (3.9 g, 11.8 mmol, 88.9% yield) as a white solid. LCMS (ESI) m/z=329 [M+H]⁺.

### Step 5: Preparation of Perfluorophenyl 5-((diethoxyphosphoryl)methyl)benzo[b]thiophene-2-carboxylate

To a cooled (0° C.) solution of 5-((diethoxyphosphoryl)methyl)benzo[b]thiophene-2-carboxylic acid (3.9 g, 11.8 mmol, 1.0 eq) in CH₂Cl₂ (50 mL) was added oxalyl chloride (2.2 g, 17.7 mmol, 1.5 eq) followed by addition of two drops of DMF. The mixture was stirred at 0° C. for 30 min, followed by evaporation of the reaction mixture to dryness. The resulting solids were dissolved in CH₂Cl₂ (50 mL), followed by addition of Et₃N (3.6 g, 35.4 mmol, 3.0 eq) and pentafluorophenol (2.6 g, 14.1 mmol, 1.2 eq). The resulting mixture was stirred at room temperature for additional 2 h and subsequently, poured over H₂O (30 mL). The bi-phasic solution was extracted with EtOAc (30 mL×3). The combined organic layers were dried over MgSO₄, filtered and concentrated under reduced pressure, the residue was purified by column chromatography on silica gel to give perfluorophenyl 5-((diethoxyphosphoryl)methyl)benzo[b]thiophene-2-carboxylate (4.7 g, 9.5 mmol, 81% yield) as a white solid. LCMS (ESI) m/z=419 [M+H]⁺; ¹H NMR (400 MHz, CDCl₃) δ 8.29 (s, 1H), 7.88 (d, J=9.1 Hz, 2H), 7.51 (d, J=8.4 Hz, 1H), 4.14-3.94 (m, 4H), 3.29 (d, J=21.5 Hz, 2H), 1.26 (t, J=7.0 Hz, 6H).

### Step 6: Preparation of ((2-((perfluorophenoxy)carbonyl)benzo[b]thiophen-5-yl)methyl)phosphonic acid

To a solution of perfluorophenyl 5-((diethoxyphosphoryl)methyl)benzo[b]thiophene-2-carboxylate (4.7 g, 9.5 mmol, 1.0 eq) in CH₂Cl₂ (60 mL) was added bromotrimethylsilane (12 mL). The mixture was stirred at room temperature for 14 h and subsequently concentrated under reduced pressure. The residue was purified by C18 column chromatography to give ((2-((perfluorophenoxy)carbonyl)benzo[b]thiophen-5-yl)methyl)phosphonic acid (3.7 g, 8.4 mmol, 89% yield) as a white solid. LCMS (ESI) m/z=439 [M+H]⁺.

### Synthesis of (R)- or (S)-5-((diethoxyphosphoryl)fluoromethyl)benzo[b]thiophene-2-carboxylic acid and (S)-5-((diethoxyphosphoryl)fluoromethyl)benzo[b]thiophene-2-carboxylic acid

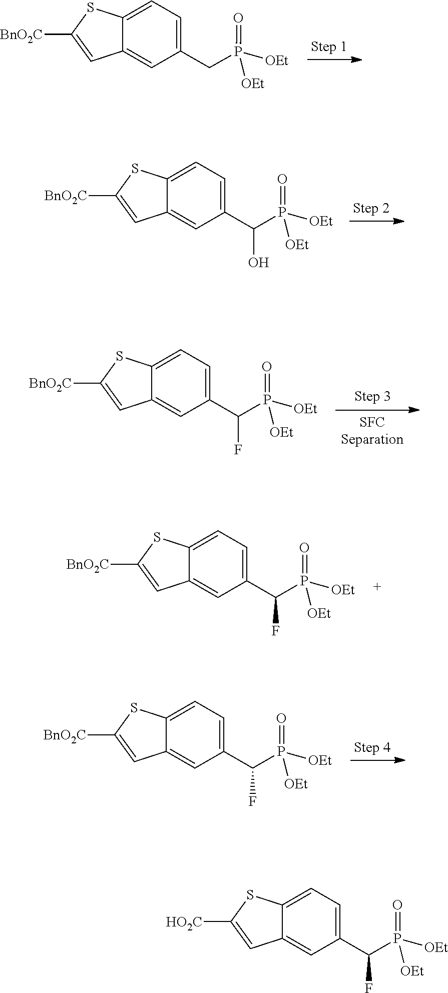

### Step 1: rac-benzyl 5-((diethoxyphosphoryl)(hydroxy)methyl)benzo[b]thiophene-2-carboxylate

To a cooled (−78° C.) solution of benzyl 5-((diethoxyphosphoryl)methyl)benzo[b]thiophene-2-carboxylate (2.4 g, 5.73 mmol, 1 eq) in THF (75 mL) and 2-(benzenesulfonyl)-3-phenyloxaziridine (2.97 g, 11.4 mmol, 2 eq) was added a 1 M solution of NaHMDS (11.4 mL, 11.4 mmol, 2 eq) in THF. A deep purple solution was observed upon addition of base that changed to orange after complete addition of the base. The mixture was stirred for an additional 10 min, followed by addition of aqueous saturated NH₄Cl (50 mL). The mixture was warmed to ambient temperatures and EtOAc (75 mL) and water (25 mL) was added. After stirring for an additional 30 min, the phases were separated. The aqueous layer was extracted with EtOAc (125 mL×2). The combined organic extracts were dried with anhydrous sodium sulfate, filtered, concentrated under reduced pressure. Another batch of equal scale was performed and combined for purification. The combined material (6.42 mmol, 12.15 mmol in total) was purified by flash chromatography (20%-100%=EtOAc: heptane) to give rac-benzyl 5-((diethoxyphosphoryl)(hydroxy)methyl)benzo[b]thiophene-2-carboxylate (3.69 g, 8.49 mmol, 70%) as a white sticky solid. LCMS (ESI) m/z=869.4 [2M+H]⁺; ¹H NMR (400 MHz, CDCl₃) δ 8.10 (s, 1H), 8.04-8.00 (m, 1H), 7.88 (d, J=8.6 Hz, 1H), 7.61 (d, J=8.6 Hz, 1H), 7.51-7.47 (m, 2H), 7.46-7.35 (m, 3H), 5.42 (s, 2H), 5.17 (dd, J=10.4, 4.5 Hz, 1H), 4.18-3.95 (m, 4H), 3.10-2.99 (m, 1H), 1.33-1.20 (m, 6H).

### Step 2: rac-benzyl 5-((diethoxyphosphoryl)fluoromethyl)benzo[b]thiophene-2-carboxylate

To a cooled (−78° C.) solution (under N₂ (g)) of rac-benzyl 5-((diethoxyphosphoryl)(hydroxy)methyl)benzo[b]thiophene-2-carboxylate (cc) (1.56 g, 3.59 mmol, 1 eq) in CH₂Cl₂ (30 mL) was added (diethylamino)sulfur trifluoride (568 μL, 4.30 mmol, 1.2 eq). The reaction was stirred for 15 min, followed by addition of aqueous saturated NaHCO₃(50 mL). After warming to room temperature, the product was extracted with CH₂Cl₂ (50 mL×3). The combined organic layers were dried over sodium sulfate, filtered and concentrated under reduced pressure. The crude residue was purified by reverse phase chromatography on C18 cartridge (eluting with 5-80% acetonitrile in water) to give rac-benzyl 5-((diethoxyphosphoryl)fluoromethyl)benzo[b]thiophene-2-carboxylate (650 mg, 1.48 mmol, 41.6%) as a thick clear oil. LCMS (ESI) m/z=873.2 [2M+H]⁺; ¹H NMR (400 MHz, CDCl₃) δ 8.12 (s, 1H), 8.02-7.99 (m, 1H), 7.92 (d, J=8.7 Hz, 1H), 7.61 (d, J=8.7 Hz, 1H), 7.51-7.51 (m, 2H), 7.46-7.36 (m, 3H), 5.82 (dd, J=44.4, 7.5 Hz, 1H), 5.42 (s, 2H), 4.21-4.02 (m, 4H), 1.34-1.26 (m, 6H).

### Step 3: Preparation of Benzyl (R)-5-((diethoxyphosphoryl)fluoromethyl)benzo[b]thiophene-2-carboxylate and benzyl (S)-5-((diethoxyphosphoryl)fluoromethyl)benzo[b]thiophene-2-carboxylate

rac-Benzyl 5-((diethoxyphosphoryl)fluoromethyl)benzo[b]thiophene-2-carboxylate (650 mg, 1.48 mmol) was submitted to chiral SFC separation (Column: Lux i-Amylose 3, 21.2×250 mm 5 um column, 75 mL/min, 40% MeOH) to give benzyl (R)- or (S)-5-((diethoxyphosphoryl)fluoromethyl)benzo[b]thiophene-2-carboxylate (304 mg, 0.70 mmol, 46.8% recovery, 99.9% ee) as a thick clear oil (Peak 1) and benzyl (R)- or (S)-5-((diethoxyphosphoryl)fluoromethyl)benzo[b]thiophene-2-carboxylate (317 mg, 0.73 mmol, 49% recovery, 99.9% ee) as a thick clear oil (Peak 2). Note: Fastest eluting enantiomer by SFC was arbitrarily assigned as (R)-5-(fluoro(phosphono)methyl)benzo[b]thiophene-2-carboxylic acid and slowest eluting enantiomer by SFC as (S)-5-(fluoro(phosphono)methyl)benzo[b]thiophene-2-carboxylic acid. HPLC method for analysis of enantiomeric excess: Lux Cellulose-3 150 mm 45% H₂O+0.05% TFA/55% MeCN 1 mL/min 8 min.

### Step 4: Preparation of (R)- or (S)-5-((diethoxyphosphoryl)fluoromethyl)benzo[b]thiophene-2-carboxylic acid

To a mixture of 10% Pd/C (60 mg, 50% wet) and benzyl (R)-5-((diethoxyphosphoryl)fluoromethyl)benzo[b]thiophene-2-carboxylate (Peak 1) (60 mg, 0.1374 mmol, 1 eq) in THF (5 mL) was degassed with N₂ (g) for 5 min. To the mixture was bubbled H₂ (g) for 5 min then the reaction was allowed to stir at room temperature under H₂ (g) (1 atm). The reaction mixture was stirred until consumption of starting material was detected by LCMS. The reaction mixture was subsequently sparged N₂ (g) for 15 min and filtered over a pad of Celite®. The filter cake was washed with 2-MeTHF and the filtrate was concentrated to give (R)- or (S)-5-((diethoxyphosphoryl)fluoromethyl)benzo[b]thiophene-2-carboxylic acid (47.4 mg, 0.0137 mmol, 99%) as a thick clear oil. LCMS (ESI) m/z=347.2 [M+H]⁺.

The following intermediate in Table 20 was prepared using the procedure outlined above (in Step 4) starting from benzyl (S)- or (R)-5-((diethoxyphosphoryl)fluoromethyl)benzo[b]thiophene-2-carboxylate (Peak 2) and using the appropriate reagents.

TABLE 20

| Name | Structure | LCMS |
|---|---|---|
| (S)-or (R)-5- ((diethoxyphosphoryl)fluoromethyl) benzo[b]thiophene-2-carboxylic acid | 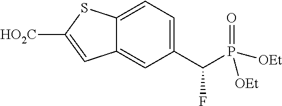 [SMILES: CCOP(=O)(OCC)[C@H](F)c1ccc2sc(C(=O)O)cc2c1] | 347.2 [M + H]⁺ |
| | or | |
| | 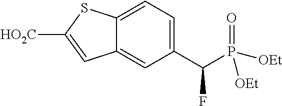 [SMILES: CCOP(=O)(OCC)[C@@H](F)c1ccc2sc(C(=O)O)cc2c1] | |

### Representative Methods for Synthesis of Activated Linkers

### Method 1: Stepwise Acid Chloride Method for Synthesis of Mixed Linkers

### Representative Procedure for the synthesis of 4-nitrophenyl 5-(((2-(butyrylthio)ethoxy)(pyridin-3-yloxy)phosphoryl)difluoromethyl)benzo[b]thiophene-2-carboxylate

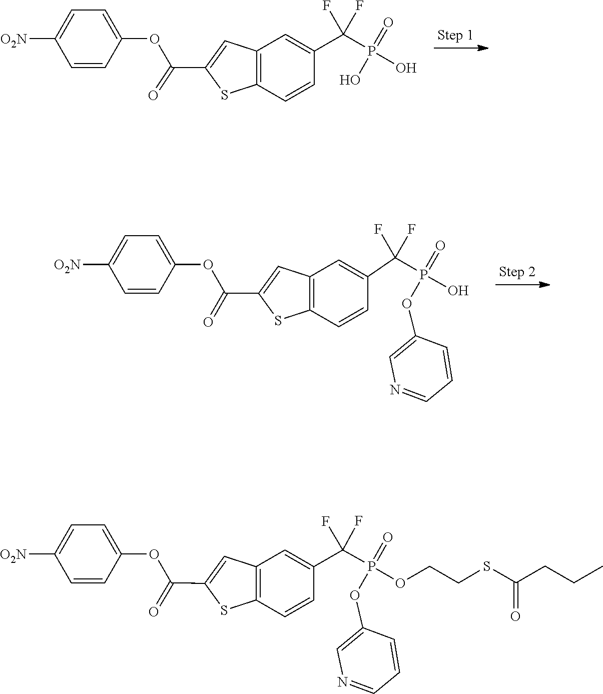

### Step 1: Preparation of 4-nitrophenyl 5-(difluoro(hydroxy(pyridin-3-yloxy)phosphoryl)methyl)benzo[b]thiophene-2-carboxylate

To a cooled (0° C.) heterogeneous solution of (difluoro(2-((4-nitrophenoxy)carbonyl)benzo[b]thiophen-5-yl)methyl)phosphonic acid (100 mg, 0.2329 mmol, 1 eq) in CH₂Cl₂ (4 mL) was added catalytic DMF (2 drops) followed by dropwise addition of oxalyl chloride (198 μL, 2.32 mmol, 10 eq). The homogenous reaction mixture was warmed up to room temperature and stirred for 2 h. The reaction was concentrated in vacuo and further dried under high vacuum for 30 min to give a yellow solid. The yellow solid was diluted in CH₂Cl₂ (4 mL) and cooled down to −78° C. A solution of pyridin-3-ol (22.0 mg, 232 μmol, 1 eq) and triethylamine (64.7 μL, 465 μmol, 2 eq) in CH₂Cl₂ (1 mL) [sonicated for 1 min to allow for solubilization] and was added slowly. The homogeneous reaction mixture was stirred at −78° C. for 2 min, then allowed to warm to ambient temperatures and stirred overnight. After 24 h, the reaction mixture turned heterogeneous and the reaction was concentrated under reduced pressure. The crude product 4-nitrophenyl 5-(difluoro(hydroxy(pyridin-3-yloxy)phosphoryl)methyl)benzo[b]thiophene-2-carboxylate was used directly in the next step without further purification or manipulation. LCMS m/z=507.2 [M+H]⁺.

### Step 2: Preparation of 4-nitrophenyl 5-(((2-(butyrylthio)ethoxy)(pyridin-3-yloxy)phosphoryl)difluoromethyl)benzo[b]thiophene-2-carboxylate

To a cooled (0° C.) solution of nitrophenyl 5-(difluoro(hydroxy(pyridin-3-yloxy)phosphoryl)methyl)benzo[b]thiophene-2-carboxylate (117 mg, 0.2329 mmol, 1 eq) in CH₂Cl₂ (5 mL) was added 2 drops of DMF followed by dropwise addition of oxalyl chloride (198 μL, 2.32 mmol, 10 eq). The reaction was warmed up to room temperature and stirred for 1.5 h. LCMS analysis showed partial conversion to desired activated intermediate. Additional oxalyl chloride (198 μL, 2.32 mmol, 10 eq) was introduced into the reaction mixture and the mixture was stirred for additional 1 h. The reaction was concentrated in vacuo and further dried under high vacuum for 30 min to give a yellow solid. The yellow solid was diluted in CH₂Cl₂ (5 mL) and cooled to −78° C. To the cooled solution was slowly added a solution of 1-[(2-hydroxyethyl)sulfanyl]butan-1-one (103 mg, 698 μmol, 3 eq) diluted in CH₂Cl₂ (1 mL) [previously dried by passing through anhydrous Na₂SO₄] followed by triethylamine (134 μL, 967 μmol, 2 eq). After stirring for 2 min, the resulting mixture was allowed to warm to ambient temperatures and stirred overnight. To the mixture was added Celite® and the mixture was carefully concentrated in vacuo. The crude residue was purified by flash-chromatography (gradient elution 0-60% EtOAc in heptanes) to give 4-nitrophenyl 5-(((2-(butyrylthio)ethoxy)(pyridin-3-yloxy)phosphoryl)difluoromethyl)benzo[b]thiophene-2-carboxylate (22.0 mg, 0.03455 mmol, 14.9% yield) as a clear oil. LCMS m/z=637.2 [M+H]⁺; ¹H NMR: (400 MHz, DMSO-d₆) δ 8.50-8.44 (m, 2H), 8.33-8.38 (m, 3H), 8.25 (s, 1H), 8.04 (d, J=8.0 Hz, 1H), 7.79 (d, J=8.6 Hz, 1H), 7.57-7.53 (m, 1H), 7.50-7.45 (m, 2H), 7.31-7.26 (m, 1H), 4.37-4.24 (m, 2H), 3.20-3.08 (m, 2H), 2.52 (t, J=7.6 Hz, 2H), 1.67 (sextet, J=7.3 Hz, 2H), 0.94 (t, J=7.6 Hz, 3H).

The following intermediates in Table 21 were prepared using a similar protocol described above for synthesis of 4-nitrophenyl 5-(((2-(butyrylthio)ethoxy)(pyridin-3-yloxy)phosphoryl)difluoromethyl)benzo[b]thiophene-2-carboxylate and utilizing the appropriate advanced intermediate(s) as starting material(s).

TABLE 21

| Name | Structure | LCMS | NMR |
|---|---|---|---|
| 4-nitrophenyl 5- (((2-(butyrylthio) ethoxy)(phenoxy) phosphoryl) difluoromethyl)benzo[b] thiophene-2-carboxylate | 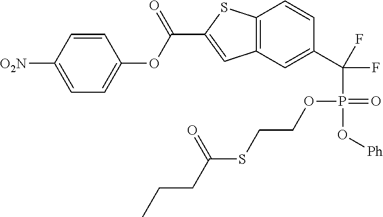 [SMILES: CCCC(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.44 (s, 1H), 8.40-8.35 (m, 3H), 7.79 (d, J = 8.8 Hz, 1H), 7.70 (d, J = 9.0 Hz, 2H), 7.44-7.37 (m, 2H), 7.29-7.23 (m, 1H), 7.18 (d, J = 8.6 Hz, 2H), 4.36-4.20 (m, 2H), 3.15 (t, J = 6.1 Hz, 2H), 2.58- 2.52 (m, 2H), 1.58-1.50 (m, 2H), 0.85 (q, J = 7.3 Hz, 3H) |
| 4-nitrophenyl 5- (((2- (butyrylthio) ethoxy) (naphthalen-1- yloxy)phosphoryl) difluoromethyl) benzo[b]thiophene- 2-carboxylate | 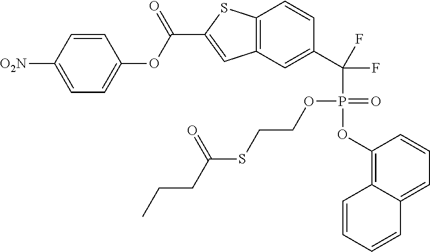 [SMILES: CCCC(=O)SCCOP(=O)(Oc1cccc2ccccc12)C(F)(F)c1ccc2sc(C(=O)Oc3ccc([N+](=O)[O-])cc3)cc2c1] | | ¹H NMR (400 MHz, DMSO-d₆) δ 8.65 (s, 1H), 8.49 (s, 1H), 8.39 (d, J = 9.3 Hz, 3H), 8.00 (d, J = 8.3 Hz, 1H), 7.87-7.80 (m, 3H), 7.70 (d, J = 9.0 Hz, 2H), 7.64-7.58 (m, 1H), 7.58-7.53 (m, 1H), 7.49 (t, J = 7.9 Hz, 1H), 7.38 (d, J = 7.6 Hz, 1H), 4.35-4.23 (m, 2H), 3.11 (t, J = 5.9 Hz, 2H), 2.45 (t, J = 7.3 Hz, 2H), 1.48 (sextet, J = 7.4 Hz, 2H), 0.81 (t, J = 7.5 Hz, 3H) |
| 4-nitrophenyl 5- (difluoro(phenoxy (2- (pivaloylthio) ethoxy)phosphoryl) methyl)benzo[b] thiophene-2- carboxylate | 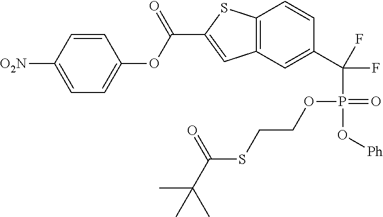 [SMILES: CC(C)(C)C(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.49-8.34 (m, 4H), 7.80 (d, J = 8.8 Hz, 1H), 7.70 (d, J = 9.0 Hz, 2H), 7.41 (t, J = 7.6 Hz, 2H), 7.30- 7.15 (m, 3H), 4.33-4.23 (m, 2H), 3.12 (t, J = 5.6 Hz, 2H), 1.12 (s, 9H) |
| 4-nitrophenyl 5- (((4- cyanophenoxy)(2- (pivaloylthio) ethoxy)phosphoryl) difluoromethyl) benzo[b]thiophene- 2-carboxylate | 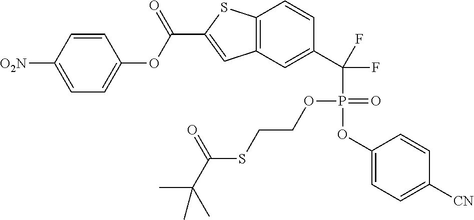 [SMILES: CC(C)(C)C(=O)SCCOP(=O)(Oc1ccc(C#N)cc1)C(F)(F)c1ccc2sc(C(=O)Oc3ccc([N+](=O)[O-])cc3)cc2c1] | | |
| 4-nitrophenyl 5- (((3- cyanophenoxy)(2- (pivaloylthio) ethoxy)phosphoryl) difluoromethyl) benzo[b]thiophene- 2-carboxylate | 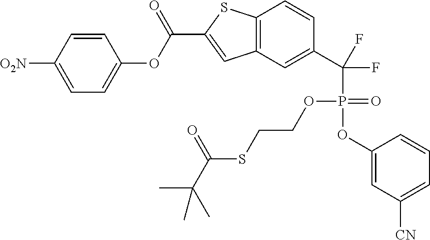 [SMILES: CC(C)(C)C(=O)SCCOP(=O)(Oc1cccc(C#N)c1)C(F)(F)c1ccc2sc(C(=O)Oc3ccc([N+](=O)[O-])cc3)cc2c1] | | ¹H NMR (400 MHz, DMSO-d₆) δ 8.66 (s, 1H), 8.46 (s, 1H), 8.42-8.34 (m, 3H), 7.83-7.67 (m, 5H), 7.66-7.59 (m, 1H), 7.58-7.53 (m, 1H), 4.40- 4.28 (m, 2H), 3.14 (t, J = 6.2 Hz, 2H), 1.12 (s, 9H) |
| 4-nitrophenyl 5- (((2-(butyrylthio) ethoxy)(3- cyanophenoxy) phosphoryl) difluoromethyl) benzo[b]thiophene- 2-carboxylate | 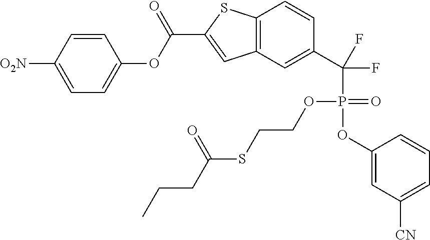 [SMILES: CCCC(=O)SCCOP(=O)(Oc1cccc(C#N)c1)C(F)(F)c1ccc2sc(C(=O)Oc3ccc([N+](=O)[O-])cc3)cc2c1] | 661.1 [M + H]⁺ | |
| 4-nitrophenyl 5- (difluoro((2-((3- methylbutanoyl) thio)ethoxy)(phenoxy) phosphoryl)methyl) benzo[b]thiophene- 2-carboxylate | 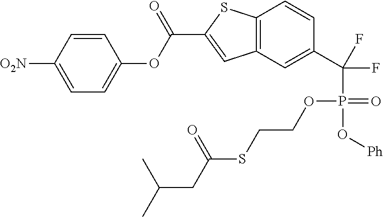 [SMILES: CC(C)CC(=O)SCCOP(=O)(Oc1ccccc1)C(F)(F)c1ccc2sc(C(=O)Oc3ccc([N+](=O)[O-])cc3)cc2c1] | | ¹H NMR (400 MHz, DMSO-d₆) δ 8.67 (s, 1H), 8.49-8.34 (m, 4H), 7.80 (d, J = 8.8 Hz, 1H), 7.70 (d, J = 9.0 Hz, 2H), 7.41 (t, J = 7.6 Hz, 2H), 7.30- 7.15 (m, 3H), 4.33-4.23 (m, 2H), 3.12 (t, J = 5.6 Hz, 2H), 1.12 (s, 9H) |
| perfluorophenyl 5-(((2-((3-((tert- butyldimethylsilyl) oxy)-2,2- dimethylpropanoyl) thio)ethoxy)(phenoxy) phosphoryl) difluoromethyl) benzo[b]thiophene- 2-carboxylate | 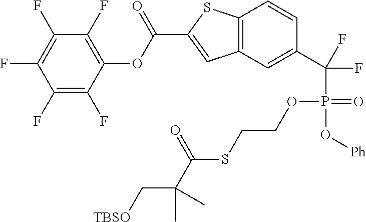 [SMILES: CC(C)(CO[Si](C)(C)C(C)(C)C)C(=O)SCCOP(=O)(Oc1ccccc1)C(F)(F)c1ccc2sc(C(=O)Oc3c(F)c(F)c(F)c(F)c3F)cc2c1] | 847.0 [M + Na]⁺ | |

### Method 2: One-Pot Acid Chloride Method for the Synthesis of Activated Linkers

### Representative Procedure for the synthesis of Perfluorophenyl 5-((bis(4-((3-methylbutanoyl)thio)butoxy)phosphoryl)difluoromethyl)benzo[b]thiophene-2-carboxylate

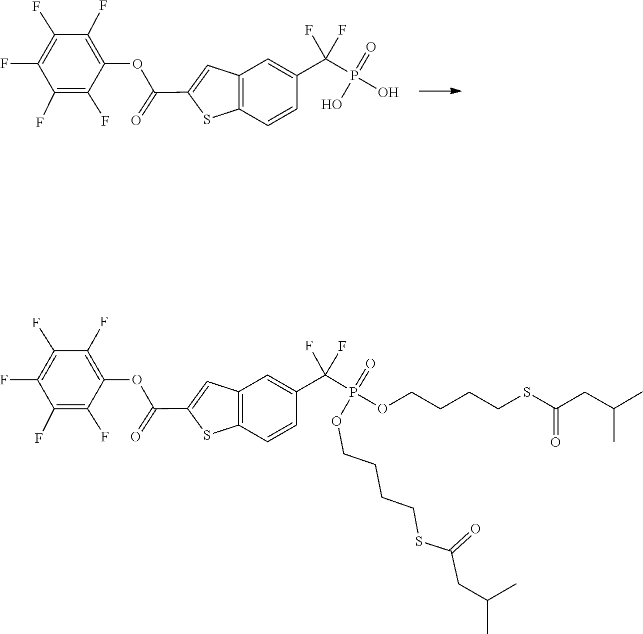 [SMILES: O=C(Oc1c(F)c(F)c(F)c(F)c1F)c1cc2cc(C(F)(F)P(=O)(O)O)ccc2s1 ; CC(C)CC(=O)SCCCCOP(=O)(OCCCCSC(=O)CC(C)C)C(F)(F)c1ccc2sc(C(=O)Oc3c(F)c(F)c(F)c(F)c3F)cc2c1]

To a cooled (0° C.) solution of 3 (200 mg, 0.42 mmol, 1.0 eq) in dry CH₂Cl₂ (15 mL) and catalytic DMF (3.2 μL, 42.1 μmol, 0.1 eq) was added in a dropwise manner oxalyl chloride (266 mg, 2.10 mmol, 5.0 eq). The reaction mixture was allowed to warm to 40° C. After stirring for 2 h, the reaction mixture was concentrated in vacuo and dried (to remove excess oxalyl chloride). The resulting solids were re-dissolved in anhydrous CH₂Cl₂ (5 mL) and cooled to 0° C. To the cooled solution was added S-(4-hydroxybutyl) 3-methylbutanethioate (239 mg, 1.26 mmol, 3.0 eq), DMAP (5.14 mg, 42.1 μmol, 0.1 eq) and a solution of N,N-diisopropylethylamine (217 mg, 1.68 mmol, 4.0 eq) in anhydrous CH₂Cl₂ (10 mL). The reaction mixture was allowed to warm to room temperature and stirred for additional 18 h. The reaction was quenched by adding H₂O (10 mL) and extracted with CH₂Cl₂ (3×10 mL). The organic layers were combined and washed with brine (20 mL), dried over anhydrous Na₂SO₄, and concentrated in vacuo. The residue was purified by flash column chromatography to afford perfluorophenyl 5-((bis(4-((3-methylbutanoyl)thio)butoxy)phosphoryl)difluoromethyl)benzo[b]thiophene-2-carboxylate (15.0 mg, 18.3 μmol, 4.4% yield). LCMS (ESI) m/z=819 [M+H]⁺.

The following intermediates in Table 22 were prepared using a similar protocol described above for synthesis of perfluorophenyl 5-((bis(4-((3-methylbutanoyl)thio)butoxy)phosphoryl)difluoromethyl)benzo[b]thiophene-2-carboxylate and utilizing the appropriate advanced intermediate(s) as starting material(s).

TABLE 22

| Name | Structure | LC-MS | NMR |
|---|---|---|---|
| 4-nitrophenyl 5- ((bis(2- (pivloylthio)ethoxy) phosphoryl)difluoro methyl)benzo[b] thiophene-2- carboxylate | 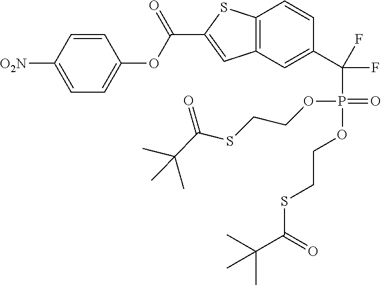 [SMILES: CC(C)(C)C(=O)SCCOP(=O)(OCCSC(=O)C(C)(C)C)C(F)(F)c1ccc2sc(C(=O)Oc3ccc([N+](=O)[O-])cc3)cc2c1] | | ¹H NMR (400 MHz, DMSO-d₆) δ 8.66 (s, 1H), 8.41-8.32 (m, 4H), 7.76- 7.65 (m, 3H), 4.22- 4.11 (m, 4H), 3.12 (t, J = 6.4 Hz, 4H), 1.16 (s, 18H) |
| perfluorophenyl 5- ((bis(2- (pivloylthio)ethoxy) phosphoryl)methyl) benzo[b]thiophene-2- carboxylate | 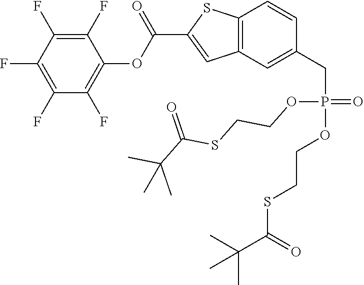 [SMILES: CC(C)(C)C(=O)SCCOP(=O)(Cc1ccc2sc(C(=O)Oc3c(F)c(F)c(F)c(F)c3F)cc2c1)OCCSC(=O)C(C)(C)C] | 749.0 [M + Na]⁺ | |

### Method 3: One-Pot Silver Salt Method for the Synthesis of Activated Linkers

### Representative procedure for the synthesis of Perfluorophenyl 5-((bis(((isopropoxycarbonyl)oxy)methoxy)phosphoryl)methyl)benzo[b]thiophene-2-carboxylate

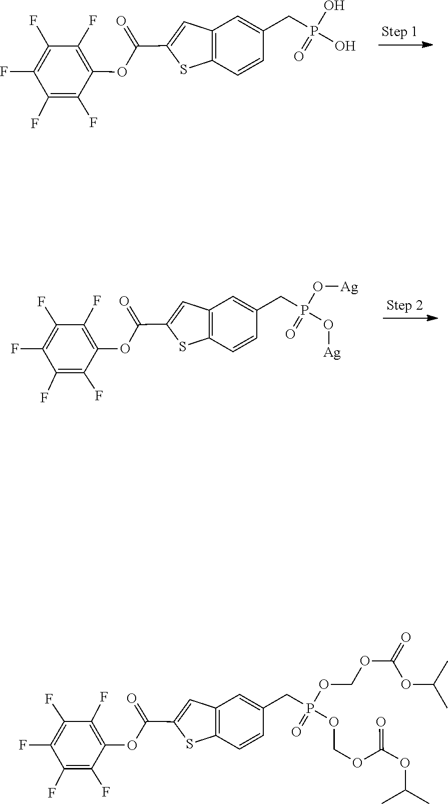

### Step 1: Preparation of Silver(I) ((2-((perfluorophenoxy)carbonyl)benzo[b]thiophen-5-yl)methyl)phosphonate

To a solution of ((2-((perfluorophenoxy)carbonyl)benzo[b]thiophen-5-yl)methyl)phosphonic acid (300 mg, 684 μmol, 1.0 eq) in a mixture of deionized H₂O (4 mL) and THF (2 mL) was added Amberlite IR120® resin (Na⁺ form) (1.5 g). The resulting mixture was stirred at room temperature for 1 h and the suspension was subsequently filtered. To the filtrate was added a solution of AgNO₃ (463 mg, 2.73 mmol, 4.0 eq) in deionized H₂O (2 mL) and the resulting mixture was stirred at room temperature for an additional 1 h. Formation of a white precipitate was observed and the solids were collected via filtration. The filter cake was then washed with cold H₂O (3×2 mL), and the solid was dried under reduced pressure to yield silver(I) ((2-((perfluorophenoxy)carbonyl)benzo[b]thiophen-5-yl)methyl)phosphonate as a dry powder. The silver salt was used without further purification.

### Step 2: Preparation of Perfluorophenyl 5-((bis(((isopropoxycarbonyl)oxy)methoxy)phosphoryl)methyl)benzo[b]thiophene-2-carboxylate

To a suspension of silver(I) ((2-((perfluorophenoxy)carbonyl)benzo[b]thiophen-5-yl)methyl)phosphonate was suspended in anhydrous toluene (10 mL) and iodomethyl 2-methylpropanoate (500 mg, 2.05 mmol, 3.0 eq) was added in a dropwise manner. After addition, the resulting mixture was stirred at room temperature for an additional 12 h. The reaction progress was monitored by LCMS, and after completion, the unreacted silver salt was recovered by filtration. The filtrate solution was concentrated in vacuo, and the resulting residue was purified by reverse phase chrormatography [C18 column gradient elution water/acetonitrile=90% to 1%] to give perfluorophenyl 5-((bis(((isopropoxycarbonyl)oxy)methoxy)phosphoryl)methyl)benzo[b]thiophene-2-carboxylate (165 mg, 246 μmol, 36% yield) as a white solid. LCMS (ESI) m/z=671 [M+H]⁺; ¹H NMR (400 MHz, CDCl₃) δ 8.28 (s, 1H), 7.96-7.80 (m, 2H), 7.48 (d, J=8.5 Hz, 1H), 5.70-5.53 (m, 4H), 4.90 (dt, J=12.6, 6.2 Hz, 2H), 3.42 (d, J=22.2 Hz, 2H), 1.31 (d, J=6.2 Hz, 12H).

The following intermediates in Table 23 were prepared using a similar protocol described above for synthesis of perfluorophenyl 5-((bis(4-((3-methylbutanoyl)thio)butoxy)phosphoryl)difluoromethyl)benzo[b]thiophene-2-carboxylate and utilizing the appropriate advanced intermediate(s) as starting material(s).

TABLE 23

| Name | Structure | LCMS | NMR |
|---|---|---|---|
| perfluorophenyl 5-((bis(2- (butyrylthio)ethoxy) phosphoryl)methyl) benzo[b]thiophene- 2-carboxylate | 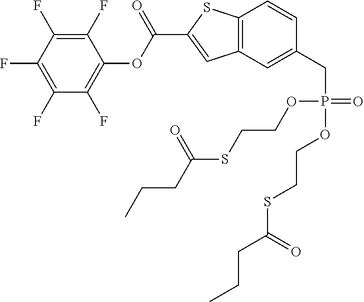 [SMILES: CCCC(=O)SCCOP(=O)(Cc1ccc2sc(C(=O)Oc3c(F)c(F)c(F)c(F)c3F)cc2c1)OCCSC(=O)CCC] | 699 [M + H]⁺ | ¹H NMR (400 MHz, CDCl₃) δ 8.31 (s, 1H), 7.96-7.80 (m, 2H), 7.50 (d, J = 8.3 Hz, 1H), 4.08- 4.03 (m, 4H), 3.33 (d, J_{HP} = 21.6 Hz, 2H), 3.08 (t, J = 6.5 Hz, 4H), 2.52 (t, J = 7.4 Hz, 4H), 1.72- 1.63 (m, 4H), 0.94 (t, J = 7.4 Hz, 6H) |
| 4-nitrophenyl 5-((bis(2- (butyrylthio)ethoxy) phosphoryl)difluoro- methyl)benzo[b]thiophene-2- carboxylate | 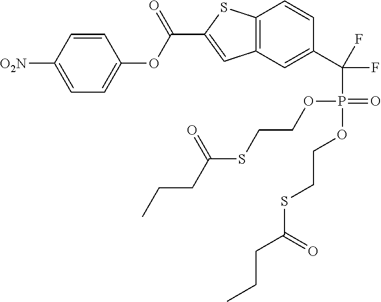 [SMILES: CCCC(=O)SCCOP(=O)(OCCSC(=O)CCC)C(F)(F)c1ccc2sc(C(=O)Oc3ccc([N+](=O)[O-])cc3)cc2c1] | 690 [M + H]⁺ | ¹H NMR (400 MHz, CDCl₃) δ 8.35-8.34 (m, 3H), 8.23 (s, 1H), 8.03 (d, J = 8.4 Hz, 1H), 7.76 (d, J = 8.7 Hz, 1H), 7.48 (d, J = 9.0 Hz, 2H), 4.29- 4.19 (m, 4H), 3.27- 3.03 (m, 4H), 2.54 (t, J = 7.4 Hz, 4H), 1.75-1.63 (m, 4H), 0.95 (t, J = 7.4 Hz, 6H) |
| perfluorophenyl 5-((bis(2-((3- methylbutanoyl) thio)ethoxy)phosphoryl) difluoromethyl) benzo[b]thiophene- 2-carboxylate | 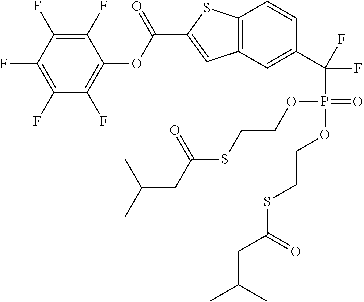 [SMILES: CC(C)CC(=O)SCCOP(=O)(OCCSC(=O)CC(C)C)C(F)(F)c1ccc2sc(C(=O)Oc3c(F)c(F)c(F)c(F)c3F)cc2c1] | | ¹H NMR (400 MHz, CDCl₃) δ 8.41 (s, 1H), 8.24 (s, 1H), 8.03 (d, J = 8.6 Hz, 1H), 7.78 (d, J = 8.7 Hz, 1H), 4.34-4.08 (m, 4H), 3.25-3.05 (m, 4H), 2.43 (d, J = 7.1 Hz, 4H), 2.14 (dt, J = 13.7, 6.8 Hz, 2H), 0.95 (s, 6H), 0.94 (s, 6H) |
| ((((2-((perfluorophenoxy) carbonyl)benzo[b]thiophen-5- yl)methyl)phosphoryl) bis(oxy))bis (methylene) bis(2,2- dimethylpropanoate) | 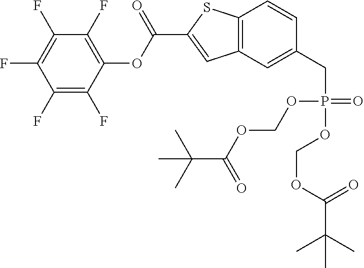 [SMILES: CC(C)(C)C(=O)OCOP(=O)(Cc1ccc2sc(C(=O)Oc3c(F)c(F)c(F)c(F)c3F)cc2c1)OCOC(=O)C(C)(C)C] | 689 [M + H]⁺ | ¹H NMR (400 MHz, CDCl₃) δ 8.28 (s, 1H), 7.90-7.86 (m, 2H), 7.50-7.45 (m, 1H), 5.68-5.56 (m, 4H), 3.38 (m, 2H), 1.19 (s, 18H) |
| (((difluoro(7-((4- nitrophenoxy) carbonyl)naphthalen-2- yl)methyl)phosphoryl) bis(oxy))bis(methylene) bis(2,2-dimethylpropanoate) | 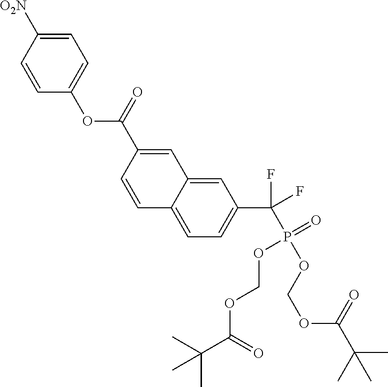 [SMILES: CC(C)(C)C(=O)OCOP(=O)(OCOC(=O)C(C)(C)C)C(F)(F)c1ccc2ccc(C(=O)Oc3ccc([N+](=O)[O-])cc3)cc2c1] | 674.1 [M + H]⁺ | ¹H NMR (400 MHz, CDCl₃) δ 8.90 (s, 1H), 8.40-8.34 (m, 2H), 8.34-8.26 (m, 2H), 8.04 (d, J = 8.4 Hz, 2H), 7.85 (d, J = 8.4 Hz, 1H), 7.52-7.47 (m, 2H), 5.81-5.66 (m, 4H), 1.21 (s, 18H) |
| perfluorophenyl 5- ((bis(2-((3-((tert- butyldimethylsilyl)oxy)-3- methylbutanoyl) thio)ethoxy)phosphoryl) difluoromethyl) benzo[b]thiophene-2- carboxylate | 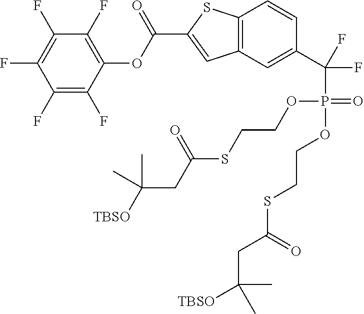 [SMILES: CC(C)(CC(=O)SCCOP(=O)(OCCSC(=O)CC(C)(C)O[Si](C)(C)C(C)(C)C)C(F)(F)c1ccc2sc(C(=O)Oc3c(F)c(F)c(F)c(F)c3F)cc2c1)O[Si](C)(C)C(C)(C)C] | | ¹H NMR (400 MHz, CDCl₃) δ 8.39-8.30 (m, 3H), 8.22 (s, 1H), 8.01 (d, J = 8.6 Hz, 1H), 7.75 (d, J = 8.5 Hz, 1H), 7.47 (d, J = 8.9 Hz, 2H), 4.25- 4.13 (m, 4H), 3.57 (s, 4H), 3.16-3.05 (m, 4H), 1.18 (s, 12H), 0.84 (s, 18H), 0.00 (s, 12H) |
| perfluorophenyl 5- ((bis(2-((3,3,3-trifluoro-2,2- dimethylpropanoyl) thio)ethoxy)phosphoryl) difluoromethyl) benzo[b]thiophene-2-carboxylate | 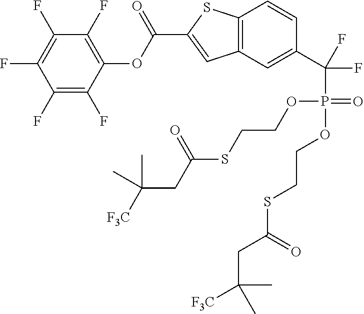 [SMILES: CC(C)(CC(=O)SCCOP(=O)(OCCSC(=O)CC(C)(C)C(F)(F)F)C(F)(F)c1ccc2sc(C(=O)Oc3c(F)c(F)c(F)c(F)c3F)cc2c1)C(F)(F)F] | 893.2 [M + Na]⁺ | |
| perfluorophenyl 7- ((bis(2-((3-methylbutanoyl) thio)ethoxy)phosphoryl) difluoromethyl)-2-naphthoate | 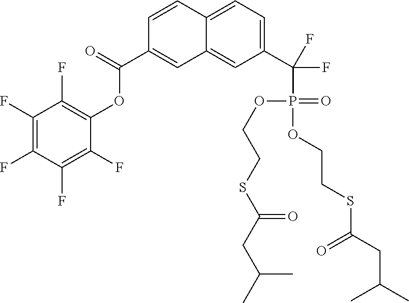 [SMILES: CC(C)CC(=O)SCCOP(=O)(OCCSC(=O)CC(C)C)C(F)(F)c1ccc2ccc(C(=O)Oc3c(F)c(F)c(F)c(F)c3F)cc2c1] | 799 [M + Na]⁺ | |
| perfluorophenyl 5- ((bis(((isopropoxy- carbonyl)oxy)methoxy) phosphoryl)methyl) benzo[b]thiophene-2- carboxylate | 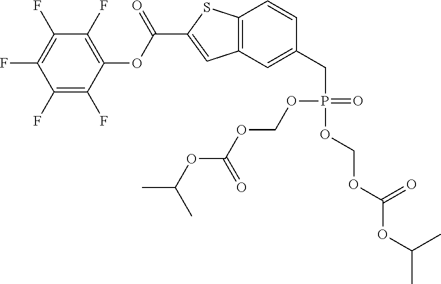 [SMILES: CC(C)OC(=O)OCOP(=O)(Cc1ccc2sc(C(=O)Oc3c(F)c(F)c(F)c(F)c3F)cc2c1)OCOC(=O)OC(C)C] | | ¹H NMR (400 MHz, CDCl₃) δ 8.28 (s, 1H), 7.96-7.80 (m, 2H), 7.48 (d, J = 8.5 Hz, 1H), 5.70-5.53 (m, 4H), 4.90 (dt, J = 12.6, 6.2 Hz, 2H), 3.42 (d, J = 22.2 Hz, 2H), 1.31 (d, J = 6.2 Hz, 12H) |
| perfluorophenyl 5- ((bis(2-((3-((tert- butyldimethylsilyl)oxy)-3- methylbutanoyl) thio)ethoxy)phosphoryl) methyl)benzo[b] thiophene-2-carboxylate | 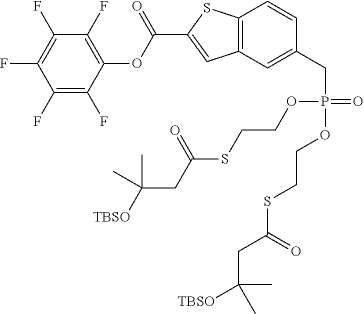 [SMILES: CC(C)(CC(=O)SCCOP(=O)(Cc1ccc2sc(C(=O)Oc3c(F)c(F)c(F)c(F)c3F)cc2c1)OCCSC(=O)CC(C)(C)O[Si](C)(C)C(C)(C)C)O[Si](C)(C)C(C)(C)C] | | ¹H NMR (400 MHz, CDCl₃) δ 8.30 (s, 1H), 7.95-7.80 (m, 2H), 7.50 (d, J = 8.4 Hz, 1H), 4.01 (dd, J = 14.7, 7.0 Hz, 4H), 3.58 (d, J = 10.6 Hz, 4H), 3.32 (d, J = 21.6 Hz, 2H), 3.04 (t, J = 6.7 Hz, 4H), 1.18 (d, J = 8.5 Hz, 12H), 0.84 (s, 18H), 0.00 (d, J = 3.5 Hz, 12H) |

### (R)- or (S)-5-((bis((pivaloyloxy)methoxy)phosphoryl)fluoromethyl)benzo[b]thiophene-2-carboxylic acid

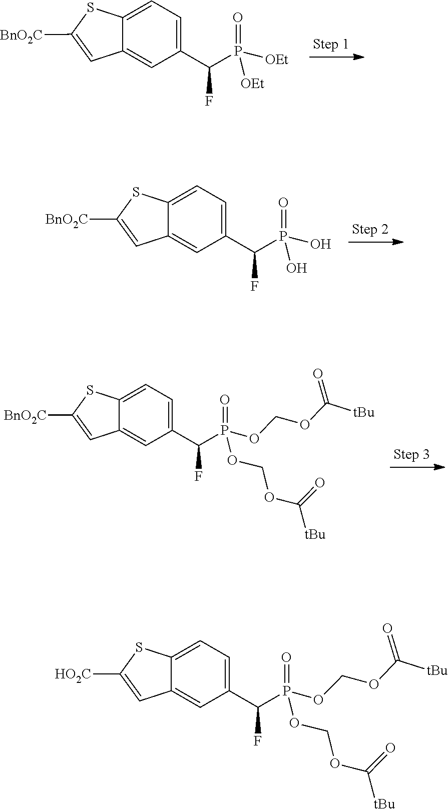

### Step 1: Preparation of (R)- or (S)-((2-((benzyloxy)carbonyl)benzo[b]thiophen-5-yl)fluoromethyl)phosphonic acid

To a cooled (0° C.) solution of benzyl (R)- or (S)-5-((diethoxyphosphoryl)fluoromethyl)benzo[b]thiophene-2-carboxylate (Peak 1) (205 mg, 0.4697 mmol) in CH₂Cl₂ (8.0 mL) was added BSTFA (746 μL, 2.81 mmol) followed by a 1.0 M solution of trimethylsilyl iodide (1.87 mL, 1.87 mmol) in CH₂Cl₂. After stirring for 1 h, a mixture of acetonitrile (0.66 mL), water (0.33 mL) and 0.1% TFA was added. The solvent was removed under reduced pressure at 0° C. The crude residue was purified by reverse phase chromatography (C18 cartridge eluting with 5-40% acetonitrile in water) to give (R)- or (S)-((2-((benzyloxy)carbonyl)benzo[b]thiophen-5-yl)fluoromethyl)phosphonic acid (163 mg, 0.4285 mmol) as a white solid. ¹H NMR (400 MHz, DMSO-d₆) δ 8.33 (s, 1H), 8.11-8.06 (m, 2H), 7.58 (d, J=8.6 Hz, 1H), 7.51-7.47 (m, 2H), 7.45-7.33 (m, 3H), 5.84 (dd, J=44.6, 8.3 Hz, 1H), 5.40 (s, 2H).

### Step 2: Preparation of (R)- or (S)-((2-((benzyloxy)carbonyl)benzo[b]thiophen-5-yl)fluoromethyl)phosphonic acid

[The following reaction was conducted in foiled covered vessel and in the absence of ambient light]: To a suspension of (R)- or (S)-((2-((benzyloxy)carbonyl)benzo[b]thiophen-5-yl)fluoromethyl)phosphonic acid (163 mg, 0.4285 mmol, 1 eq) in water (5 mL) was added a solution of aqueous sodium hydroxide (34.2 mg, 857 μmol, 2 eq) in water (2 mL). To the yellow solution was added silver(I) nitrate (181 mg, 1.07 mmol, 2.5 eq) and the resulting off-white suspension was stirred for 1.5 h at room temperature. The suspension was cooled to 0° C., filtered, and dried under high vacuum. The solids were re-suspended in acetonitrile, concentrated under reduced pressure, and further dried under high vacuum (3 h). The resulting dark yellow powder was suspended in toluene (10 mL) and iodomethyl 2,2-dimethylpropanoate (191 μL, 1.28 mmol, 3 eq) was added. After stirring for 20 h, the reaction mixture was stirred for 20 h at room temperature. The reaction mixture was filtered and rinsed with toluene. The filtrate was concentrated under reduced pressure. The crude residue was purified (C18 cartridge eluting with 5-100% acetonitrile in water) to give (R)- or (S)-((((2-((benzyloxy)carbonyl)benzo[b]thiophen-5-yl)fluoromethyl)phosphoryl)bis(oxy))bis(methylene)bis(2,2-dimethylpropanoate) (122 mg, 0.2004 mmol, 46.9%) as a clear oil. ¹H NMR (400 MHz, CDCl₃) δ 8.11 (s, 1H), 8.01-7.99 (m, 1H), 7.92 (d, J=8.3 Hz, 1H), 7.59 (d, J=8.3 Hz, 1H), 7.52-7.47 (m, 2H), 7.46-7.36 (m, 3H), 8.87 (dd, J=44.3, 7.5 Hz, 1H), 5.72-5.62 (m, 4H), 5.43 (s, 2H), 1.21 (s, 18H).

### Step 3: Preparation of (R)- or (S)-5-((bis((pivaloyloxy)methoxy)phosphoryl)fluoromethyl)benzo[b]thiophene-2-carboxylic acid

To a solution of (R)-((((2-((benzyloxy)carbonyl)benzo[b]thiophen-5-yl)fluoromethyl)phosphoryl)bis(oxy))bis(methylene)bis(2,2-dimethylpropanoate) (122 mg, 0.200 mmol, 1 eq) in THF (10 mL) under N₂ (g) was added 10% Pd/C (50% wet, 120 mg, 0.1127 mmol, 0.56 eq). To the suspension was bubbled H₂ (g) for 5 min. The reaction mixture was stirred under H₂ (g) (1 atm) at room temperature. After stirring for 22 h, the reaction mixture was purged with N₂ (g) and filtered over Celite®. The filter pad was washed with THF and concentrated under reduced pressure to give (R)-5-((bis((pivaloyloxy)methoxy)phosphoryl)fluoromethyl)benzo[b]thiophene-2-carboxylic acid (0.200 mmol, 99.9%) as a thick clear oil. LC-MS (ESI) m/z [M+H]⁺=519.1.

The following intermediates in Table 24 were prepared using a similar protocol described above for synthesis of (R)- or (S)-5-((bis((pivaloyloxy)methoxy)phosphoryl)fluoromethyl)benzo[b]thiophene-2-carboxylic acid and utilizing the appropriate advanced intermediate(s) as starting material(s). The absolute configuration of the starting material, (R)- or (S)-((2-((benzyloxy)carbonyl)benzo[b]thiophen-5-yl)fluoromethyl)phosphonic acid was not determined, but the elution peak (“Peak 1” or “Peak 2”) of the starting material utilized is indicated in the table

TABLE 24

| Name | Structure | LCMS | NMR |
|---|---|---|---|
| (R)- or (S)-5- ((bis((pivaloyloxy) methoxy)phosphoryl) fluoromethyl)benzo [b]thiophene-2- carboxylic acid | 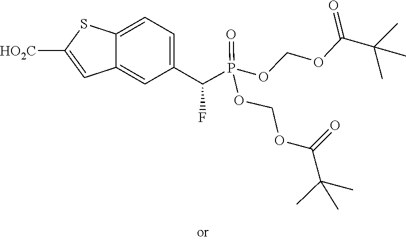 [SMILES: CC(C)(C)C(=O)OCOP(=O)(OCOC(=O)C(C)(C)C)[C@H](F)c1ccc2sc(C(=O)O)cc2c1] | 519.1 [M + H]⁺ | |
| | 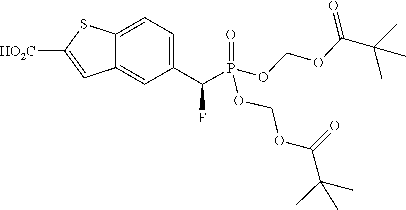 [SMILES: CC(C)(C)C(=O)OCOP(=O)(OCOC(=O)C(C)(C)C)[C@@H](F)c1ccc2sc(C(=O)O)cc2c1] | | |
| | (Peak 2 of phosphonate | | |
| | building block was utilized for | | |
| | synthesis) | | |
| (R)- or (S)-5- ((bis(((isopropoxy- carbonyl)oxy) methoxy) phosphoryl)fluoro- methyl)benzo[b] thiophene-2- carboxylic acid | 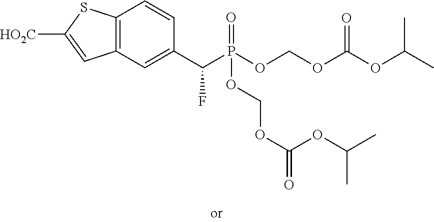 | 689 [M + H]⁺ | ¹H NMR (400 MHz, CDCl₃) δ 8.35 (s, 1H), 8.09 (s, 1H), 7.97 (d, J = 8.5 Hz, 1H), 7.65 (d, J = 8.6 Hz, 1H), 5.93 (dd, J = 44.2, 7.3 Hz, 1H), 5.73-5.54 (m, 4H), 4.97-4.84 (m, 2H), 1.33-1.29 (m, 12H) |
| | 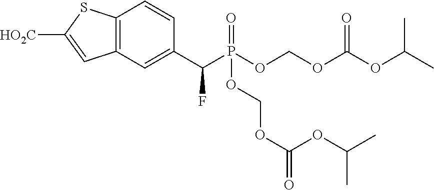 [SMILES: CC(C)OC(=O)OCOP(=O)(OCOC(=O)OC(C)C)[C@@H](F)c1ccc2sc(C(=O)O)cc2c1] | | |
| | (Peak 2 of phosphonate | | |
| | building block was utilized for | | |
| | synthesis) | | |

### Method 4: Stepwise Silver Salt Method for the Synthesis of Mixed Linkers

### Representative procedure for the Perfluorophenyl 5-(((2-(butyrylthio)ethoxy)(2-(pivaloylthio)ethoxy)phosphoryl)difluoromethyl)benzo[b]thiophene-2-carboxylate

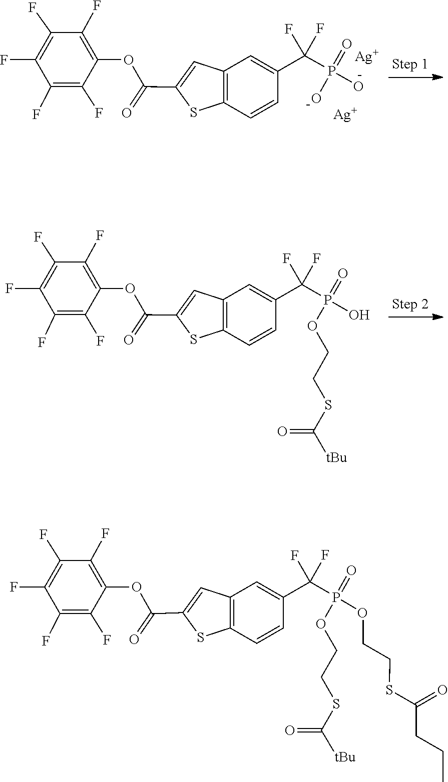

### Step 1: Preparation of Perfluorophenyl 5-(difluoro(hydroxy(2-(pivaloylthio)ethoxy)phosphoryl)methyl)benzo[b]thiophene-2-carboxylate

Silver(I) (difluoro(2-((perfluorophenoxy)carbonyl)benzo[b]thiophen-5-yl)methyl)phosphonate was synthesized starting from (difluoro(2-((perfluorophenoxy)carbonyl)benzo[b]thiophen-5-yl)methyl)phosphonic acid using the method described in Step 1, Method 3 for the synthesis of silver(I) ((2-((perfluorophenoxy)carbonyl)benzo[b]thiophen-5-yl)methyl)phosphonate.

To a suspension of silver(I) ((2-((perfluorophenoxy)carbonyl)benzo[b]thiophen-5-yl)methyl)phosphonate (647 mg, 940 μmol, 1.0 eq) in anhydrous toluene (10 mL) was added in a dropwise manner S-(2-iodoethyl) 2,2-dimethylpropanethioate (310 mg, 1.14 mmol, 1.2 eq). After complete addition of the alcohol, the resulting mixture was stirred at room temperature for an additional 12 h. The heterogeneous mixture was filtered, and the filtrate was concentrated in vacuo. The resulting residue was purified by reverse phase chromatography to give perfluorophenyl 5-(difluoro(hydroxy(2-(pivaloylthio)ethoxy)phosphoryl)methyl)benzo[b]thiophene-2-carboxylate (240 mg, 388 μmol, 41% yield). LCMS (ESI) m/z=617 [M−H]⁻.

### Step 2: Preparation of Perfluorophenyl 5-(((2-(butyrylthio)ethoxy)(2-(pivaloylthio)ethoxy)phosphoryl)difluoromethyl)benzo[b]thiophene-2-carboxylate

Perfluorophenyl 5-(((2-(butyrylthio)ethoxy)(2-(pivaloylthio)ethoxy)phosphoryl)difluoromethyl)benzo[b]thiophene-2-carboxylate was synthesized using similar protocol outlined above. Starting with perfluorophenyl 5-(difluoro(hydroxy(2-(pivaloylthio)ethoxy)phosphoryl)methyl)benzo[b]thiophene-2-carboxylate (190 mg, 307 μmol, 1.0 eq), AgNO₃ (207 mg, 1.22 mmol, 4.0 eq), and S-(2-iodoethyl) butanethioate (94.9 mg, 368 μmol, 1.2 eq) produced perfluorophenyl 5-(((2-(butyrylthio)ethoxy)(2-(pivaloylthio)ethoxy)phosphoryl)difluoromethyl)benzo[b]thiophene-2-carboxylate (55.0 mg, 73.4 μmol, 24%) as a white solid. LCMS (ESI) m/z=749 [M+H]⁺.

The following intermediates in Table 25 were prepared using a similar protocol described above for synthesis of perfluorophenyl 5-(((2-(butyrylthio)ethoxy)(2-(pivaloylthio)ethoxy)phosphoryl)difluoromethyl)benzo[b]thiophene-2-carboxylate and utilizing the appropriate advanced intermediate(s) as starting material(s).

TABLE 25

| Name | Structure | LCMS | NMR |
|---|---|---|---|
| perfluorophenyl 5- (difluoro (((isopropoxycarbonyl) oxy)methoxy)(2-((3- methylbutanoyl) thio)ethoxy)phosphoryl) methyl)benzo[b] thiophene-2- carboxylate | 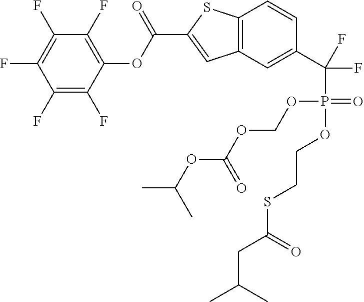 [SMILES: CC(C)CC(=O)SCCOP(=O)(OCOC(=O)OC(C)C)C(F)(F)c1ccc2sc(C(=O)Oc3c(F)c(F)c(F)c(F)c3F)cc2c1] | 757 [M + Na]⁺ | ¹H NMR (400 MHz, CDCl₃) δ 8.40 (s, 1H), 8.24 (s, 1H), 8.03 (d, J = 8.6 Hz, 1H), 7.77 (d, J = 8.7 Hz, 1H), 5.69 (ddd, J = 17.8, 12.2, 5.2 Hz, 2H), 4.93 (dt, J = 12.5, 6.3 Hz, 1H), 4.34- 4.14 (m, 2H), 3.14 (td, J = 6.6, 3.0 Hz, 2H), 2.42 (d, J = 7.1 Hz, 2H), 2.13 (dt, J = 13.7, 6.8 Hz, 1H), 1.33 (s, 3H), 1.31 (s, 3H), 0.95 (s, 3H), 0.95 (s, 3H) |

### Preparation of 5-(((((S)-1-isopropoxy-1-oxopropan-2-yl)amino)(phenoxy)phosphoryl)methyl)benzo[b]thiophene-2-carboxylic acid

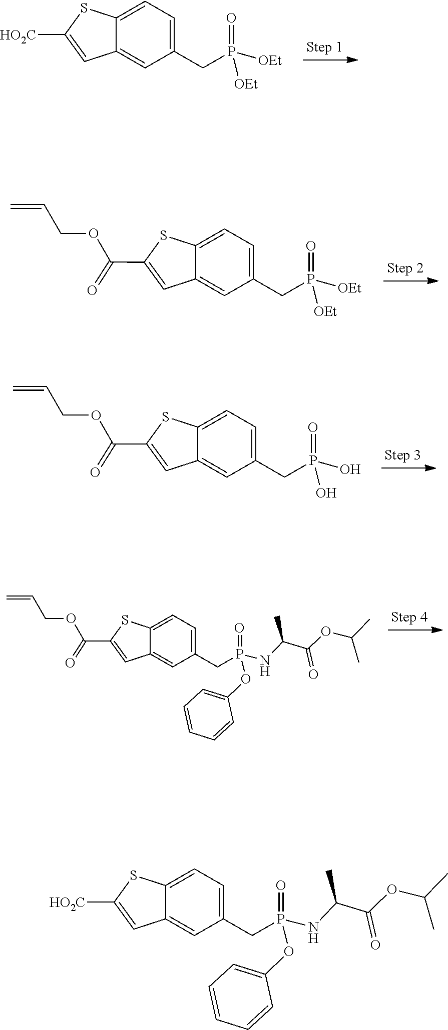 [SMILES: CCOP(=O)(Cc1ccc2sc(C(=O)O)cc2c1)OCC ; C=CCOC(=O)c1cc2cc(CP(=O)(OCC)OCC)ccc2s1 ; C=CCOC(=O)c1cc2cc(CP(=O)(O)O)ccc2s1 ; C=CCOC(=O)c1cc2cc(CP(=O)(N[C@@H](C)C(=O)OC(C)C)Oc3ccccc3)ccc2s1 ; CC(C)OC(=O)[C@H](C)NP(=O)(Cc1ccc2sc(C(=O)O)cc2c1)Oc1ccccc1]

### Step 1: Preparation of Allyl 5-((diethoxyphosphoryl)methyl)benzo[b]thiophene-2-carboxylate

To a suspension of 5-((diethoxyphosphoryl)methyl)benzo[b]thiophene-2-carboxylic acid (1.0 g, 3.0 mmol, 1.0 eq) and K₂CO₃ (839 mg, 6.1 mmol, 2.0 eq) in DMF (20 mL) was added 3-bromoprop-1-ene (440 mg, 3.6 mmol, 1.2 eq). The mixture was stirred at room temperature for 14 h and poured over water (30 mL). The mixture was extracted with EtOAc (25 mL×3). The combined organic layers were dried over Na₂SO₄, filtered, and concentrated under reduced pressure, the residue was purified by column chromatography to give allyl 5-((diethoxyphosphoryl)methyl)benzo[b]thiophene-2-carboxylate (0.980 g, 2.7 mmol, 88% yield) as a light-yellow solid. LCMS (ESI) m/z=369 [M+H]⁺; ¹H NMR (400 MHz, CDCl₃) δ 8.04 (s, 1H), 7.84-7.77 (m, 2H), 7.45-7.37 (m, 1H), 6.18-5.94 (m, 1H), 5.49-5.39 (m, 1H), 5.36-5.28 (m, 1H), 4.87-4.83 (m, 2H), 4.08-3.97 (m, 4H), 3.27 (d, J=21.4 Hz, 2H), 1.25 (t, J=7.1 Hz, 6H).

### Step 2: Preparation of ((2-((allyloxy)carbonyl)benzo[b]thiophen-5-yl)methyl)phosphonic acid

To a solution of allyl 5-((diethoxyphosphoryl)methyl)benzo[b]thiophene-2-carboxylate (980 mg, 2.7 mmol, 1.0 eq) in CH₂Cl₂ (15 mL) was added bromotrimethylsilane (3 mL). The mixture was stirred at room temperature for 14 h and subsequently concentrated under reduced pressure. The resulting residue was triturated with H₂O (5 mL) and the resulting precipitates were filtered. The filter cake was washed with H₂O (5 mL×2) and dried under reduced pressure to give ((2-((allyloxy)carbonyl)benzo[b]thiophen-5-yl)methyl)phosphonic acid (0.710 g, 2.3 mmol, 86% yield) as a white solid. LCMS (ESI) m/z=313 [M+H]⁺; ¹H NMR (400 MHz, DMSO-d₆) δ 8.20 (s, 1H), 7.96 (d, J=8.4 Hz, 1H), 7.92-7.83 (m, 1H), 7.50-7.38 (m, 1H), 6.12-5.98 (m, 1H), 5.46-5.38 (m, 1H), 5.32-5.27 (m, 1H), 4.85-4.80 (m, 2H), 3.08 (d, J=21.2 Hz, 2H).

### Step 3: Preparation of Allyl 5-(((((S)-1-isopropoxy-1-oxopropan-2-yl)amino)(phenoxy)phosphoryl)methyl)benzo[b]thiophene-2-carboxylate

To a cooled (0° C.) solution (under a constant stream of N₂ (g)) of ((2-((allyloxy)carbonyl)benzo[b]thiophen-5-yl)methyl)phosphonic acid (2.80 g, 8.96 mmol, 1 eq) and catalytic DMF (1 drop) in dry CH₂Cl₂ (50 mL) was added oxalyl chloride (3.40 g, 26.8 mmol, 3 eq). After effervescence of gas ceased, the mixture was warmed at 40° C. After 2 h, the mixture was cooled to room temperature and concentrated in vacuo to give yellow solids. The solids were subsequently diluted CH₂Cl₂ (50 mL) and cooled to 0° C. To the cooled solution was added phenol (0.843 g, 8.96 mmol, 1 eq) and Et₃N (4.53 g, 44.8 mmol, 5 eq). After complete addition, the mixture was warmed to room temperature and stirred for 1 h, followed by introduction of propan-2-yl (2S)-2-aminopropanoate (1.75 g, 13.4 mmol, 1.5 eq) to the mixture. After stirring for an additional 2 h, the mixture was concentrated to dryness. The residue was purified by C18 column (elution 50%-80% acetonitrile in water) to give allyl 5-(((((S)-1-isopropoxy-1-oxopropan-2-yl)amino)(phenoxy)phosphoryl)methyl)benzo[b]thiophene-2-carboxylate (2.23 g, 4.44 mmol, 49.6% yield) as white solids. LCMS (ESI) m/z=502.0 [M+H]⁺; ¹H NMR (400 MHz, CDCl₃) δ 8.05 (d, J=6.9 Hz, 1H), 7.91-7.80 (m, 2H), 7.53-7.43 (m, 1H), 7.29 (d, J=8.1 Hz, 2H), 7.18-7.09 (m, 3H), 6.05 (ddd, J=16.1, 10.9, 5.6 Hz, 1H), 5.48-5.40 (m, 1H), 5.32 (dd, J=10.4, 1.2 Hz, 1H), 4.98-4.87 (m, 1H), 4.85 (d, J=5.7 Hz, 2H), 4.04-3.85 (m, 1H), 3.44 (dd, J=20.7, 14.1 Hz, 2H), 3.12 (t, J=10.9 Hz, 1H), 1.21-1.10 (m, 9H).

### Step 4: Preparation of 5-(((((S)-1-isopropoxy-1-oxopropan-2-yl)amino)(phenoxy)phosphoryl)methyl)benzo[b]thiophene-2-carboxylic acid

A solution of allyl 5-(((((S)-1-isopropoxy-1-oxopropan-2-yl)amino)(phenoxy)phosphoryl)methyl)benzo[b]thiophene-2-carboxylate (90 mg, 0.1794 mmol, 1 eq), pyrrolidine (12.7 mg, 179 μmol, 1 eq), Pd(PPh₃)₄(10.3 mg, 8.97 μmol, 0.05 eq) in CH₂Cl₂ (5 mL) was stirred under N₂ (g). After 2 h, the reaction was concentrated in vacuo. The residue was purified by C18 column (elution 30%-70% acetonitrile in water) to yield 5-(((((S)-1-isopropoxy-1-oxopropan-2-yl)amino)(phenoxy)phosphoryl)methyl)benzo[b]thiophene-2-carboxylic acid (64.0 mg, 0.1386 mmol, 77.4% yield) as white solids. LCMS (ESI) m/z=462.1 [M+H]⁺.

The following intermediates in Table 26 were prepared using the described above for synthesis of 5-(((((S)-1-isopropoxy-1-oxopropan-2-yl)amino)(phenoxy)phosphoryl)methyl)benzo[b]thiophene-2-carboxylic acid and utilizing the appropriate utilizing the appropriate starting materials and modifications.

TABLE 26

| Name | Structure | LCMS | NMR |
|---|---|---|---|
| 5-(fluoro((((S)-1- isopropoxy-1- oxopropan-2- yl)amino)(phenoxy) phosphoryl)methyl) benzo[b]thiophene-2- carboxylic acid | 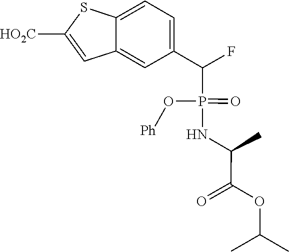 [SMILES: CC(C)OC(=O)[C@H](C)NP(=O)(Oc1ccccc1)C(F)c1ccc2sc(C(=O)O)cc2c1] | 480.1 [M + H]⁺ | |

### Syntheses of Phosphonic Acid Analogues

Method 1: Representative procedure for the Synthesis of Phosphonic Acid Analogues via Amino Acid Coupling of Carboxylic Acid Linkers with Amino Acid Cores.

### (Difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonic acid (1)

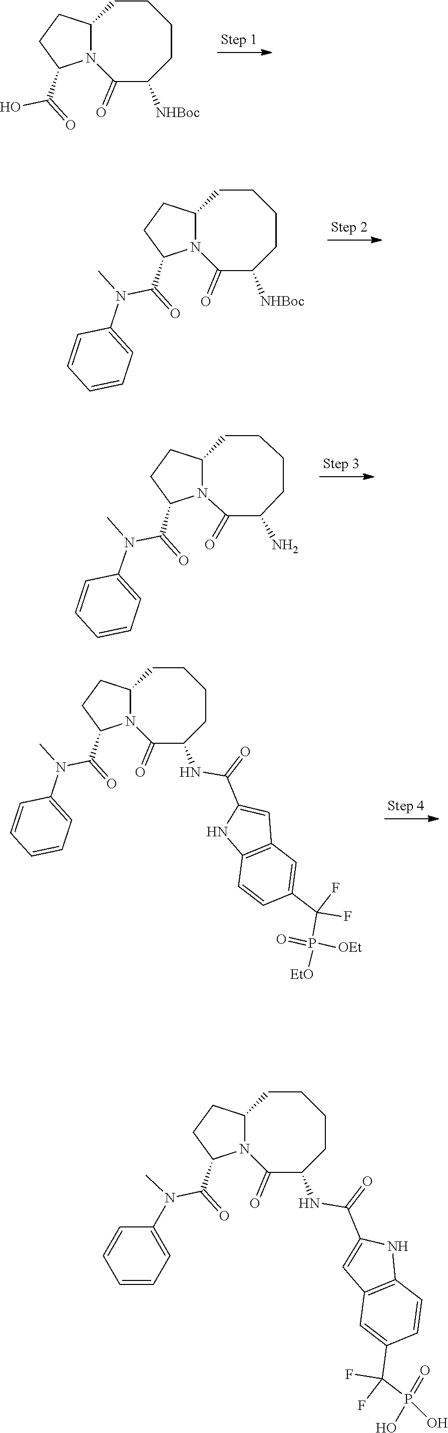

### Step 1: Preparation of Tert-Butyl ((3S,6S,10aS)-3-(methyl(phenyl)carbamoyl)-5-oxodecahydropyrrolo[1,2-a]azocin-6-yl)carbamate

To a solution of (3S,6S,10aS)-6-((tert-butoxycarbonyl)amino)-5-oxodecahydropyrrolo[1,2-a]azocine-3-carboxylic acid (400 mg, 1.22 mmol, 1.0 eq), N-methylaniline (261 mg, 2.44 mmol, 2.0 eq) and Et₃N (246 mg, 2.44 mmol, 2.0 eq) in CH₂Cl₂ (10 mL) was added T₃P (1.55 g, 2.44 mmol, 2 eq). The resulting mixture was stirred for 12 h at 40° C. The reaction mixture was diluted with water (5 mL) and extracted with CH₂Cl₂ (10 mL×3). The organic layers were combined and washed with brine (20 mL), dried over with anhydrous Na₂SO₄, and concentrated under reduced pressure. The resulting residue was purified by flash column chromatography to afford tert-butyl ((3S,6S,10aS)-3-(methyl(phenyl)carbamoyl)-5-oxodecahydropyrrolo[1,2-a]azocin-6-yl)carbamate (240 mg, 578 μmol, 47% yield) as a white solid. LCMS (ESI) m/z=416 [M+H]⁺.

### Step 2: Preparation of (3S,6S,10aS)-6-amino-N-methyl-5-oxo-N-phenyldecahydropyrrolo[1,2-a]azocine-3-carboxamide

To a solution of tert-butyl ((3S,6S,10aS)-3-(methyl(phenyl)carbamoyl)-5-oxodecahydropyrrolo[1,2-a]azocin-6-yl)carbamate (100 mg, 240 μmol, 1 eq) in CH₂Cl₂ (6 mL) was added trifluoroacetic acid (3 mL). The reaction mixture was stirred for 1 h at room temperature, subsequently cooled to 0° C., and neutralized carefully with aqueous NaHCO₃ until basic (adjusted to pH=8-9). The resulting mixture was then extracted with CH₂Cl₂ (10 mL×3 mL), and the combined organic layers were washed with brine (2×10 mL), dried over with anhydrous Na₂SO₄, and concentrated under reduced pressure to give crude (3S,6S,10aS)-6-amino-N-methyl-5-oxo-N-phenyldecahydropyrrolo[1,2-a]azocine-3-carboxamide (76 mg, 240 μmol) as a white solid, which was used in the next step directly without further purification. LCMS (ESI) m/z=316 [M+H]⁺.

### Step 3: Preparation of Diethyl (difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonate

To a solution of (3S,6S,10aS)-6-amino-N-methyl-5-oxo-N-phenyldecahydropyrrolo[1,2-a]azocine-3-carboxamide (76 mg, 240 μmol, 1.0 eq) and 5-[(diethoxyphosphoryl)difluoromethyl]-1H-indole-2-carboxylic acid (83.3 mg, 240 μmol, 1.0 eq) in DMF (3 mL) was added HATU (118 mg, 312 μmol, 1.3 eq) and Et₃N (72.8 mg, 720 μmol, 3.0 eq). The resulting mixture was stirred for an additional 2 h at room temperature. The reaction mixture was diluted with H₂O (10 mL) and extracted with CH₂Cl₂ (10 mL×3). The organic layers were combined and washed with brine (20 mL), dried over with anhydrous Na₂SO₄, and concentrated in vacuo. The resulting residue was purified by flash column chromatography to afford diethyl (difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonate (100 mg, 155 μmol, 65% yield) as a white solid. LCMS (ESI) m/z=645 [M+H]⁺.

### Step 4: Preparation of (Difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonic acid (1)

To a cooled (0° C.) solution of diethyl (difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonate (100 mg, 155 μmol, 1.0 eq) in CH₂Cl₂ (10 mL) was added in a dropwise manner bromotrimethylsilane (355 mg, 2.32 mmol, 15.0 eq). The reaction was allowed to warm to room temperature and stirred. After 12 h, the reaction mixture was quenched by addition of H₂ (5 mL). The biphasic mixture was extracted using with CH₂NM₂ (3×10 mL). The organic layers were combined and washed with brine (20 mL), dried over with anhydrous Na₂SO₄, and concentrated in vacuo. The resulting residue was purified by reverse phase HPLC to afford (difluoro(2-(((3S,6S,10as)-3-(methyl(phenyl)carbamoyl)-5-oxodecahydropyrrolo[1,2-a]azocin-6-yl)carbamoyl)-1H-indol-5-yl)methyl)phosphonic acid (1) (8.70 mg, 14.7 μmol, 9.5% yield) as a white solid. LCMS (ESI) m/z=589 [M+H]; ¹H NMR (400 MHz, DMSO-d₆) δ 11.74 (s, 1H), 8.51 (d, J 7.1 Hz, 1H), 7.82 (s, 1H), 7.41 (m, 8H), 4.96 (d, J 5.4 Hz, 1H), 4.17 (m, 4H), 3.16 (s, 3H), 1.93 (m, 8H), 1.57 (in, 4H)

The following compounds in Table 27 were prepared according to the representative procedure described above for the synthesis of (difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonic acid (1) and utilizing the appropriate starting materials and modifications.

TABLE 27

| Com- | | | | |
|---|---|---|---|---|
| pound | Name | Structure | LCMS | NMR |
| 2 | (difluoro(2- (((3S,6S,9R,10aR)-9- hydroxy-3-(3- (morpholine-4- carbonyl)azetidine-1- carbonyl)-5- oxodecahydropyrrolo [1,2-a]azocin-6- yl)carbamoyl)benzo[b] thiophen-5-yl)methyl) phosphonic acid | 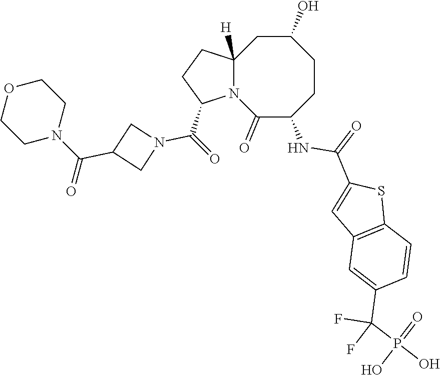 [SMILES: O=C(N[C@H]1CC[C@@H](O)C[C@H]2CC[C@@H](C(=O)N3CC(C(=O)N4CCOCC4)C3)N2C1=O)c1cc2cc(C(F)(F)P(=O)(O)O)ccc2s1] | 685.3 [M + H]⁺ | ¹H NMR (400 MHz, methanol-d₄) δ 8.17- 8.09 (m, 2H), 8.04-7.95 (m, 1H), 7.70-7.62 (m, 1H), 4.87-4.81 (m, 1H), 4.71-4.62 (m, 1H), 4.51- 4.36 (m, 3H), 4.32- 4.20 (m, 1H), 4.14-4.04 (m, 1H), 3.97-3.88 (m, 1H), 3.83-3.71 (m, 1H), 3.68-3.43 (m, 6H), 3.35 (t, J = 4.8 Hz, 1H), 3.30- 3.23 (m, 1H), 2.45-2.20 (m, 3H), 2.20-1.84 (m, 6H), 1.84-1.75 (m, 1H) |
| 3 | (difluoro(2- (((3S,6S,8R, 10aR)-8-hydroxy-3- (3-(morpholine- 4-carbonyl)azetidine-1- carbonyl)-5-oxodeca- hydropyrrolo[1,2- a]azocin-6-yl) carbamoyl) benzo[b]thiophen-5- yl)methyl)phosphonic acid | 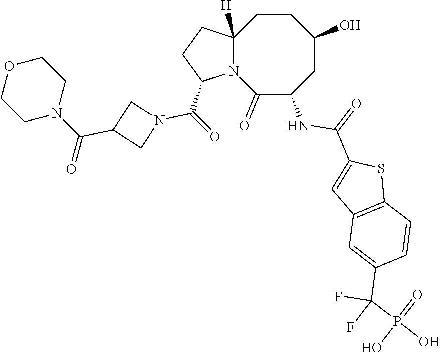 [SMILES: O=C(N[C@H]1C[C@H](O)CC[C@H]2CC[C@@H](C(=O)N3CC(C(=O)N4CCOCC4)C3)N2C1=O)c1cc2cc(C(F)(F)P(=O)(O)O)ccc2s1] | 685.2 [M + H]⁺ | ¹H NMR (400 MHz, methanol-d₄) δ 8.21- 8.11 (m, 2H), 8.06-7.97 (m, 1H), 7.72-7.63 (m, 1H), 5.08-4.97 (m, 1H), 4.71-4.61 (m, 1H), 4.49- 4.34 (m, 3H), 4.32- 4.21 (m, 2H), 4.15-4.02 (m, 1H), 3.86-3.74 (m, 1H), 3.69-3.46 (m, 6H), 3.41-3.35 (m, 1H), 3.35- 3.32 (m, 1H), 2.35- 2.18 (m, 3H), 2.17-1.80 (m, 6H), 1.79-1.70 (m, 1H) |
| 4 | (difluoro(2- (((3S,6S,8S,10aR)-8- hydroxy-3-(3- (morpholine- 4-carbonyl)azetidine- 1-carbonyl)-5- oxodecahydropyrrolo [1,2-a]azocin-6- yl)carbamoyl) benzo[b]thiophen-5- yl)methyl) phosphonic acid | 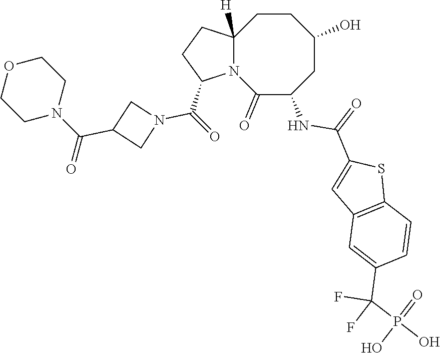 [SMILES: O=C(N[C@H]1C[C@@H](O)CC[C@H]2CC[C@@H](C(=O)N3CC(C(=O)N4CCOCC4)C3)N2C1=O)c1cc2cc(C(F)(F)P(=O)(O)O)ccc2s1] | 685.3 [M + H]⁺ | ¹H NMR (400 MHz, methanol-d₄) δ 8.15- 8.14 (m, 2H), 8.03-8.00 (m, 1H), 7.68 (d, J = 8.8 Hz, 1H), 4.97-4.89 (m, 1H), 4.66 (t, J = 8.3 Hz, 1H), 4.46-4.31 (m, 3H), 4.34-4.23 (m, 1H), 4.15- 4.05 (m, 1H), 4.04- 3.96 (m, 1H), 3.85-3.74 (m, 1H), 3.68-3.48 (m, 6H), 3.40-3.32 (m, 2H), 2.35-2.14 (m, 4H), 2.13- 1.83 (m, 5H), 1.82- 1.71 (m, 1H) |
| 5 | (difluoro(2- (((3S,6S,9S,10aR)-9- hydroxy-3-(3- (morpholine-4- carbonyl)azetidine-1- carbonyl)-5- oxodecahydropyrrolo [1,2-a]azocin-6- yl)carbamoyl) benzo[b]thiophen-5- yl)methyl)phosphonic acid | 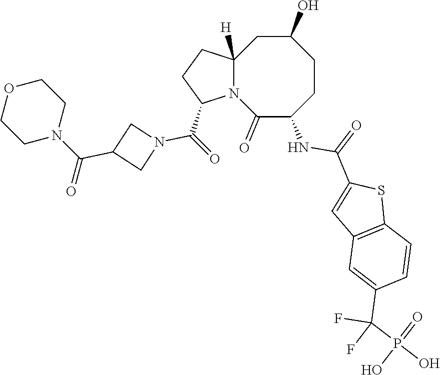 [SMILES: O=C(N[C@H]1CC[C@H](O)C[C@H]2CC[C@@H](C(=O)N3CC(C(=O)N4CCOCC4)C3)N2C1=O)c1cc2cc(C(F)(F)P(=O)(O)O)ccc2s1] | 685.1 [M + H]⁺ | ¹H NMR (400 MHz, methanol-d₄) δ 8.18- 8.11 (m, 2H), 8.05-7.97 (m, 1H), 7.71-7.63 (m, 1H), 5.21-5.08 (m, 1H), 4.74-4.57 (m, 2H), 4.51- 4.37 (m, 2H), 4.32- 4.20 (m, 2H), 4.14-4.04 (m, 1H), 3.84-3.73 (m, 1H), 3.69-3.47 (m, 6H), 3.41-3.32 (m, 2H), 2.34- 2.10 (m, 5H), 2.09- 1.92 (m, 2H), 1.89-1.70 (m, 3H) |
| 6 | ((2- (((3S,6S,9R,10aR)-9- (benzyloxy)-3-(3- (morpholine-4- carbonyl)azetidine-1- carbonyl)-5- oxodecahydropyrrolo [1,2-a]azocin-6- yl)carbamoyl)benzo[b] thiophen-5-yl) difluoromethyl) phosphonic acid | 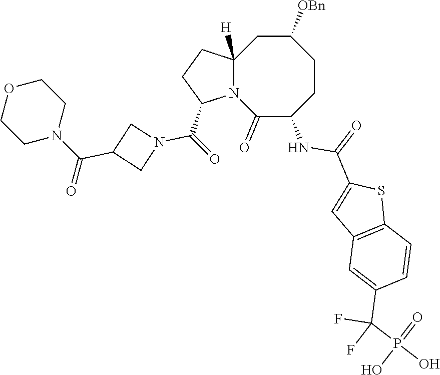 [SMILES: O=C(N[C@H]1CC[C@@H](OCc2ccccc2)C[C@H]2CC[C@@H](C(=O)N3CC(C(=O)N4CCOCC4)C3)N2C1=O)c1cc2cc(C(F)(F)P(=O)(O)O)ccc2s1] | 775.3 [M + H]⁺ | ¹H NMR (400 MHz, methanol-d₄) δ 8.17- 8.09 (m, 2H), 8.05-7.95 (m, 1H), 7.71-7.63 (m, 1H), 7.40-7.22 (m, 5H), 4.86-4.78 (m, 1H), 4.71- 4.63 (m, 1H), 4.63- 4.51 (m, 2H), 4.49-4.37 (m, 3H), 4.31-4.20 (m, 1H), 4.13-4.05 (m, 1H), 3.83-3.44 (m, 9H), 3.38- 3.34 (m, 1H), 2.43- 2.22 (m, 3H), 2.22-2.10 (m, 1H), 2.08-1.87 (m, 6H) |
| 7 | ((2- (((3S,6S,8R, 10aR)-8- (benzyloxy)-3-(3- (morpholine-4- carbonyl)azetidine-1- carbonyl)-5- oxodecahydropyrrolo [1,2-a]azocin-6- yl)carbamoyl) benzo[b]thiophen-5- yl)difluoromethyl) phosphonic acid | 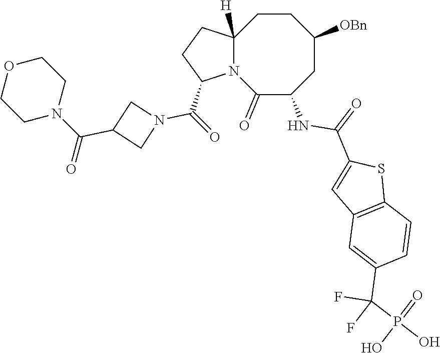 [SMILES: O=C(N[C@H]1C[C@H](OCc2ccccc2)CC[C@H]2CC[C@@H](C(=O)N3CC(C(=O)N4CCOCC4)C3)N2C1=O)c1cc2cc(C(F)(F)P(=O)(O)O)ccc2s1] | 775.3 [M + H]⁺ | ¹H NMR (400 MHz, methanol-d₄) δ 8.19- 8.11 (m, 2H), 8.06-7.99 (m, 1H), 7.72-7.64 (m, 1H), 7.40-7.20 (m, 5H), 5.13-5.02 (m, 1H), 4.73- 4.64 (m, 1H), 4.62- 4.51 (m, 2H), 4.49-4.36 (m, 3H), 4.34-4.23 (m, 1H), 4.22-4.06 (m, 2H), 3.85-3.74 (m, 1H), 3.69- 3.52 (m, 6H), 3.40- 3.33 (m, 2H), 2.44-2.32 (m, 1H), 2.31-1.95 (m, 7H), 1.92-1.72 (m, 2H) |
| 8 | ((2- (((3S,6S,8S,10aR)-8- (benzyloxy)-3-(3- (morpholine-4- carbonyl)azetidine-1- carbonyl)-5- oxodecahydropyrrolo [1,2-a]azocin-6- yl)carbamoyl)benzo [b]thiophen-5- yl)difluoromethyl) phosphonic acid | 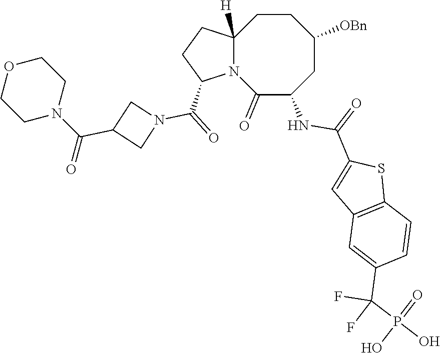 [SMILES: O=C(N[C@H]1C[C@@H](OCc2ccccc2)CC[C@H]2CC[C@@H](C(=O)N3CC(C(=O)N4CCOCC4)C3)N2C1=O)c1cc2cc(C(F)(F)P(=O)(O)O)ccc2s1] | 775.3 [M + H]⁺ | ¹H NMR (400 MHz, methanol-d₄) δ 8.15 (s, 2H), 8.02 (d, J = 8.8 Hz, 1H), 7.68 (d, J = 8.6 Hz, 1H), 7.39-7.22 (m, 5H), 4.85-4.81 (m, 1H), 4.70 (t, J = 8.3 Hz, 1H), 4.63- 4.53 (m, 2H), 4.46-4.36 (m, 2H), 4.34-4.19 (m, 2H), 4.13-4.03 (m, 1H), 3.82-3.73 (m, 1H), 3.72- 3.53 (m, 7H), 3.39- 3.32 (m, 2H), 2.46-2.43 (m, 1H), 2.32-1.93 (m, 6H), 1.90-1.67 (m, 3H) |
| 9 | ((2- (((3S,6S,9R,10aR)-9- ethoxy-3-(3- (morpholine-4- carbonyl)azetidine-1- carbonyl)-5- oxodecahydropyrrolo [1,2-a]azocin-6- yl)carbamoyl) benzo[b]thiophen-5- yl)difluoromethyl) phosphonic acid | 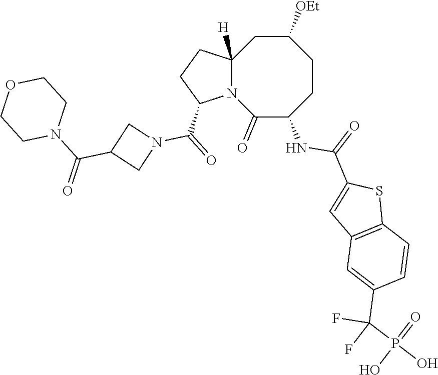 [SMILES: CCO[C@@H]1CC[C@H](NC(=O)c2cc3cc(C(F)(F)P(=O)(O)O)ccc3s2)C(=O)N2[C@H](CC[C@H]2C(=O)N2CC(C(=O)N3CCOCC3)C2)C1] | 713.2 [M + H]⁺ | ¹H NMR (400 MHz, methanol-d₄) δ 8.19- 8.09 (m, 2H), 8.05-7.97 (m, 1H), 7.72-7.64 (m, 1H), 4.73-4.62 (m, 1H), 4.53-4.36 (m, 3H), 4.31- 4.19 (m, 1H), 4.14- 4.03 (m, 1H), 3.86-3.74 (m, 1H), 3.69-3.46 (m, 10H), 3.41-3.33 (m, 2H), 2.38-2.21 (m, 3H), 2.15-1.82 (m, 7H), 1.18 (t, J = 7.0 Hz, 3H) |
| 10 | ((2- (((3S,6S,8S,10aR)-8- ethoxy-3-(3- (morpholine-4- carbonyl)azetidine-1- carbonyl)-5- oxodecahydropyrrolo [1,2-a]azocin-6- yl)carbamoyl) benzo[b]thiophen-5- yl)difluoromethyl) phosphonic acid | 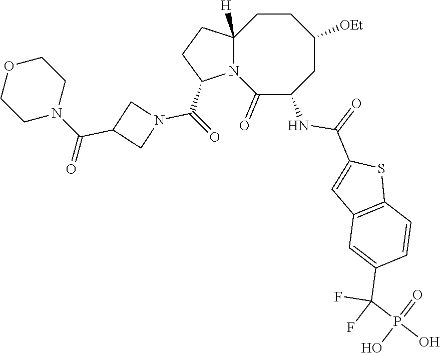 [SMILES: CCO[C@H]1CC[C@H]2CC[C@@H](C(=O)N3CC(C(=O)N4CCOCC4)C3)N2C(=O)[C@@H](NC(=O)c2cc3cc(C(F)(F)P(=O)(O)O)ccc3s2)C1] | 713.2 [M + H]⁺ | ¹H NMR (400 MHz, methanol-d₄) δ 8.16- 8.14 (m, 2H), 8.02 (d, J = 8.6 Hz, 1H), 7.68 (d, J = 8.3 Hz, 1H), 4.73-4.66 (m, 1H), 4.46-4.21 (m, 4H), 4.13-4.04 (m, 1H), 3.83-3.73 (m, 1H), 3.68- 3.46 (m, 10H), 3.40- 3.32 (m, 2H), 2.40-2.12 (m, 3H), 2.07-1.71 (m, 7H), 1.21-1.15 (m, 3H) |
| 11 | ((2- (((3S,6S,8R,10aR)-8- ethoxy-3-(3- (morpholine-4- carbonyl)azetidine-1- carbonyl)-5- oxodecahydropyrrolo [1,2-a]azocin-6-yl) carbamoyl)benzo[b] thiophen-5- yl)difluoromethyl) phosphonic acid | 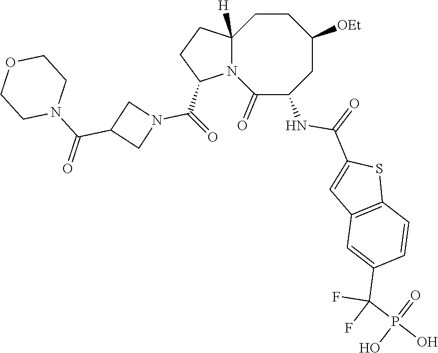 [SMILES: CCO[C@@H]1CC[C@H]2CC[C@@H](C(=O)N3CC(C(=O)N4CCOCC4)C3)N2C(=O)[C@@H](NC(=O)c2cc3cc(C(F)(F)P(=O)(O)O)ccc3s2)C1] | 713.3 [M + H]⁺ | ¹H NMR (400 MHz, methanol-d₄) δ 8.20- 8.10 (m, 2H), 8.02 (d, J = 8.6 Hz, 1H), 7.68 (d, J = 8.3 Hz, 1H), 5.09-4.98 (m, 1H), 4.72-4.62 (m, 1H), 4.49-4.35 (m, 3H), 4.32-4.22 (m, 1H), 4.16- 4.05 (m, 1H), 4.04- 3.94 (m, 1H), 3.86-3.72 (m, 1H), 3.69-3.45 (m, 8H), 3.41-3.33 (m, 2H), 2.39-2.15 (m, 3H), 2.12- 1.72 (m, 7H), 1.19 (t, J = 7.1 Hz, 3H) |
| 12 | ((2- (((3S,6S,8S,10aR)-3- ((3R,4S)-3-cyano-4- phenylpyrrolidine-1- carbonyl)-8- hydroxy-5- oxodecahydropyrrolo [1,2-a]azocin-6- yl)carbamoyl)benzo[b] thiophen-5-yl) difluoromethyl) phosphonic acid | 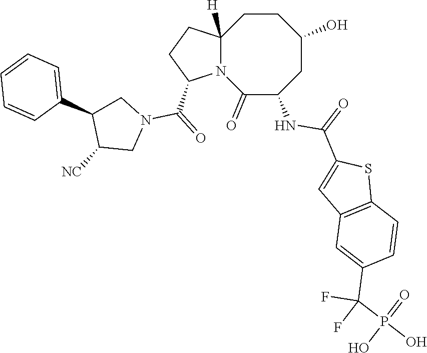 [SMILES: N#C[C@H]1CN(C(=O)[C@@H]2CC[C@@H]3CC[C@H](O)C[C@H](NC(=O)c4cc5cc(C(F)(F)P(=O)(O)O)ccc5s4)C(=O)N32)C[C@@H]1c1ccccc1] | 687.2 [M + H]⁺ | ¹H NMR (400 MHz, methanol-d₄) δ 8.22- 8.11 (m, 2H), 8.07-7.96 (m, 1H), 7.67 (d, J = 8.6 Hz, 1H), 7.50-7.25 (m, 5H), 5.02-4.92 (m, 1H), 4.67-4.56 (m, 1H), 4.47- 4.34 (m, 1H), 4.24- 4.15 (m, 1H), 4.15-3.86 (m, 3H), 3.83-3.56 (m, 2H), 3.55-3.39 (m, 1H), 2.51-2.18 (m, 4H), 2.17- 2.06 (m, 1H), 2.05- 1.84 (m, 4H), 1.83-1.69 (m, 1H) |
| 13 | (difluoro(2- (((3S,6S,9S,10aR)-9- hydroxy-9-methyl-3- (3-(morpholine-4- carbonyl)azetidine-1- carbonyl)-5- oxodecahydropyrrolo [1,2-a]azocin-6- yl)carbamoyl)benzo [b]thiophen-5-yl) methyl) phosphonic acid | 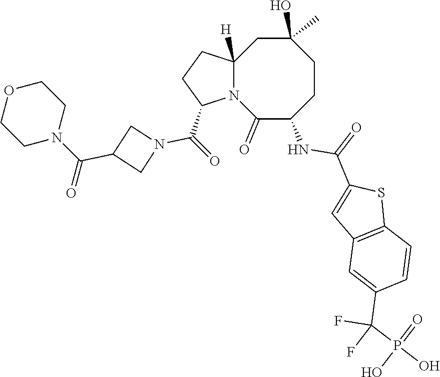 [SMILES: C[C@]1(O)CC[C@H](NC(=O)c2cc3cc(C(F)(F)P(=O)(O)O)ccc3s2)C(=O)N2[C@H](CC[C@H]2C(=O)N2CC(C(=O)N3CCOCC3)C2)C1] | 699.3 [M + H]⁺ | ¹H NMR (400 MHz, methanol-d₄) δ 8.15- 8.11 (m, 2H), 8.03-7.96 (m, 1H), 7.66 (d, J = 7.8 Hz, 1H), 5.32-5.23 (m, 1H), 4.72-4.57 (m, 2H), 4.50-4.36 (m, 2H), 4.30- 4.20 (m, 1H), 4.13- 4.05 (m, 1H), 3.83-3.73 (m, 1H), 3.67-3.46 (m, 6H), 3.38-3.32 (m, 2H), 2.38-2.10 (m, 5H), 2.04- 1.89 (m, 1H), 1.85- 1.72 (m, 2H), 1.71-1.55 (m, 2H), 1.28 (s, 3H) |
| 14 | ((2- (((3S,6S,8R,10aR)-3- ((3R,4S or 3S,4R)-3- cyano-4- phenylpyrrolidine-1- carbonyl)-8- hydroxy-8-methyl-5- oxodecahydropyrrolo [1,2-a]azocin-6- yl)carbamoyl)benzo[b] thiophen-5-yl) difluoromethyl) phosphonic acid | 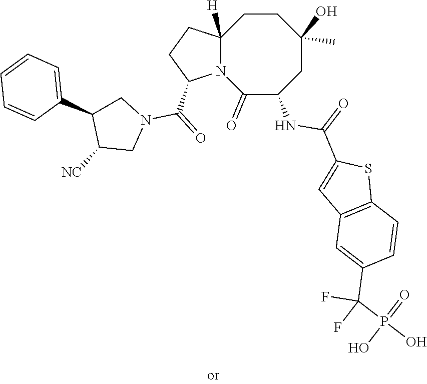 [SMILES: C[C@@]1(O)CC[C@H]2CC[C@@H](C(=O)N3C[C@H](c4ccccc4)[C@@H](C#N)C3)N2C(=O)[C@@H](NC(=O)c2cc3cc(C(F)(F)P(=O)(O)O)ccc3s2)C1] | 701.2 [M + H]⁺ | ¹H NMR (400 MHz, methanol-d₄) δ 8.18- 8.13 (m, 2H), 8.06-7.99 (m, 1H), 7.68 (d, J = 8.6 Hz, 1H), 7.50-7.26 (m, 5H), 5.18-5.09 (m, 1H), 4.66-4.54 (m, 2H), 4.23- 4.14 (m, 0.8H), 4.13- 4.05 (m, 1.2H), 3.92 (t, J = 10.1 Hz, 0.5H), 3.83- 3.75 (m, 0.5H), 3.75- 3.70 (m, 0.7H), 3.67- 3.57 (m, 1.3H), 3.52- 3.37 (m, 1H), 2.44-2.33 (m, 1H), 2.33-2.13 (m, 2H), 2.10-1.93 (m, 3.5H), 1.90-1.73 (m, 2.5H), 1.71-1.60 (m, 1H), 1.36-1.30 (m, 3H) |
| | | 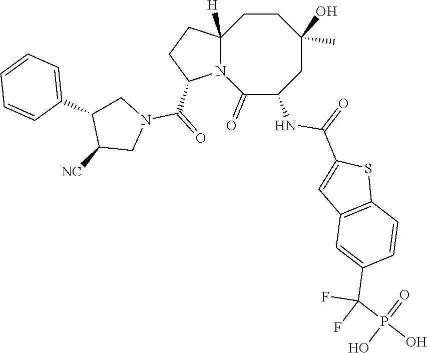 [SMILES: C[C@@]1(O)CC[C@H]2CC[C@@H](C(=O)N3C[C@@H](C#N)[C@H](c4ccccc4)C3)N2C(=O)[C@@H](NC(=O)c2cc3cc(C(F)(F)P(=O)(O)O)ccc3s2)C1] | | |
| | | SFC peak 1 was used for | | |
| | | biological testing | | |
| 15 | ((2- (((3S,6S,9S,10aR)- 3-(3- (1H-imidazol-1-yl) azetidine-1-carbonyl)- 9-hydroxy-5- oxodecahydropyrrolo [1,2-a]azocin-6- yl)carbamoyl)benzo[b] thiophen-5-yl) difluoromethyl) phosphonic acid | 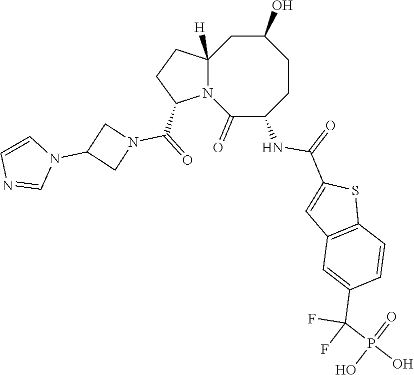 [SMILES: O=C(N[C@H]1CC[C@H](O)C[C@H]2CC[C@@H](C(=O)N3CC(n4ccnc4)C3)N2C1=O)c1cc2cc(C(F)(F)P(=O)(O)O)ccc2s1] | 638.2 [M + H]⁺ | ¹H NMR (400 MHz, methanol-d₄) δ 9.09- 9.03 (m, 1H), 8.22-8.16 (m, 1H), 8.16-8.11 (m, 1H), 8.05-7.99 (m, 1H), 7.95-7.87 (m, 1H), 7.76- 7.69 (m, 1H), 7.58- 7.53 (m, 1H), 5.43-5.32 (m, 1H), 5.20-5.08 (m, 1H), 4.81-4.70 (m, 1H), 4.69-4.60 (m, 1H), 4.56- 4.16 (m, 5H), 2.35- 1.69 (m, 10H) |
| 16 | (difluoro(2- (((3S,6S,9S,10aR)-9- hydroxy-5-oxo-3-(3- (pyridin-3-yl) azetidine-1-carbonyl) decahydropyrrolo[1,2- a]azocin-6-yl) carbamoyl)benzo[b] thiophen-5-yl)methyl) phosphonic acid | 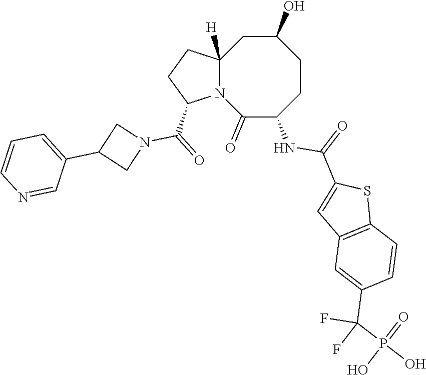 [SMILES: O=C(N[C@H]1CC[C@H](O)C[C@H]2CC[C@@H](C(=O)N3CC(c4cccnc4)C3)N2C1=O)c1cc2cc(C(F)(F)P(=O)(O)O)ccc2s1] | 649.2 [M + H]⁺ | ¹H NMR (400 MHz, methanol-d₄) δ 8.69- 8.40 (m, 2H), 8.25-7.99 (m, 3H), 7.97-7.87 (m, 1H), 7.78-7.69 (m, 1H), 7.64-7.47 (m, 1H), 5.22- 5.09 (m, 1H), 5.05- 4.91 (m, 1H), 4.75-4.59 (m, 2H), 4.58-4.35 (m, 2H), 4.34-4.22 (m, 1H), 4.11-3.94 (m, 2H), 2.38- 2.12 (m, 5H), 2.11- 1.96 (m, 2H), 1.95-1.71 (m, 3H) |
| 17 | (difluoro(2- (((3S,6S,8R,10aR)-8- hydroxy-8-methyl-3- (3-(morpholine-4- carbonyl)azetidine-1- carbonyl)-5- oxodecahydropyrrolo [1,2-a]azocin-6-yl) carbamoyl)benzo[b] thiophen-5-yl)methyl) phosphonic acid | 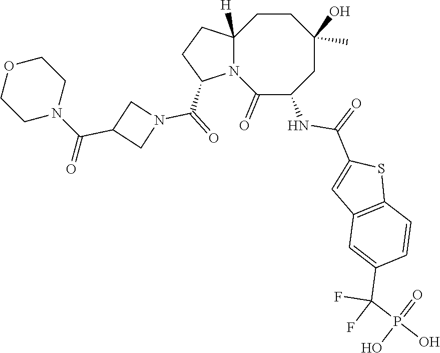 [SMILES: C[C@@]1(O)CC[C@H]2CC[C@@H](C(=O)N3CC(C(=O)N4CCOCC4)C3)N2C(=O)[C@@H](NC(=O)c2cc3cc(C(F)(F)P(=O)(O)O)ccc3s2)C1] | 699.3 [M + H]⁺ | ¹H NMR (400 MHz, methanol-d₄) δ 8.16- 8.10 (m, 2H), 8.03-7.96 (m, 1H), 7.69-7.64 (m, 1H), 5.15-5.09 (m, 1H), 4.71-4.64 (m, 1H), 4.59- 4.51 (m, 1H), 4.48- 4.35 (m, 2H), 4.32-4.20 (m, 1H), 4.12-4.05 (m, 1H), 3.82-3.72 (m, 1H), 3.67-3.43 (m, 6H), 3.37- 3.33 (m, 1H), 3.29- 3.20 (m, 1H), 2.30-2.10 (m, 3H), 2.08-1.91 (m, 4H), 1.85-1.58 (m, 3H), 1.31 (s, 3H) |
| 18 | ((2- (((3S,6S,10aS)-3- ((3S,4R or 3R,4S)-3- cyano-4- phenylpyrrolidine- 1- carbonyl)-5- oxodecahydropyrrolo [1,2-a]azocin-6- yl)carbamoyl) benzo[b]thiophen-5- yl)fluoromethyl) phosphonic acid | 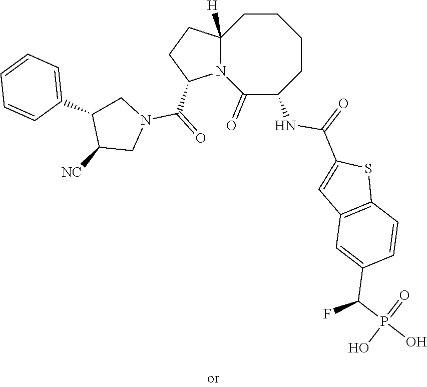 [SMILES: N#C[C@@H]1CN(C(=O)[C@@H]2CC[C@@H]3CCCC[C@H](NC(=O)c4cc5cc([C@H](F)P(=O)(O)O)ccc5s4)C(=O)N32)C[C@H]1c1ccccc1] | 653.1 [M + H]⁺ | ¹H NMR (400 MHz, methanol-d₄) δ 8.11 (s, 1H), 8.07-8.00 (m, 1H), 7.98-7.91 (m, 1H), 7.64- 7.55 (m, 1H), 7.45-7.27 (m, 5H), 5.82 (m, 1H), 5.09-4.97 (m, 1H), 4.74-4.63 (m, 1H), 4.58- 4.38 (m, 2H), 4.13- 3.91 (m, 1H), 3.90-3.64 (m, 2H), 3.63-3.46 (m, 2H), 2.43-2.16 (m, 2H), 2.13-1.93 (m, 6H), 1.91- 1.75 (m, 2H), 1.73- 1.55 (m, 2H) |
| | | 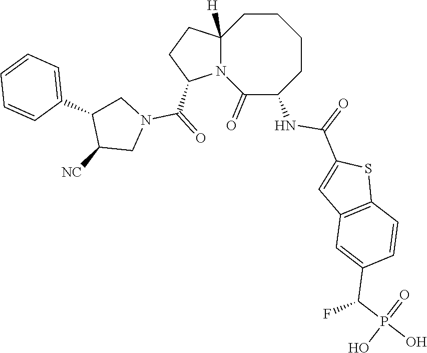 [SMILES: N#C[C@@H]1CN(C(=O)[C@@H]2CC[C@@H]3CCCC[C@H](NC(=O)c4cc5cc([C@@H](F)P(=O)(O)O)ccc5s4)C(=O)N32)C[C@H]1c1ccccc1] | | |
| | | SFC peak 1 was used for | | |
| | | biological testing | | |
| 19 | ((2- (((3S,6S,10aS)-3- ((3S,4R or 3R,4S))- 3-cyano-4- phenylpyrrolidine-1- carbonyl)-5- oxodecahydropyrrolo [1,2-a]azocin-6- yl)carbamoyl) benzo[b]thiophen-5- yl)fluoromethyl) phosphonic acid | 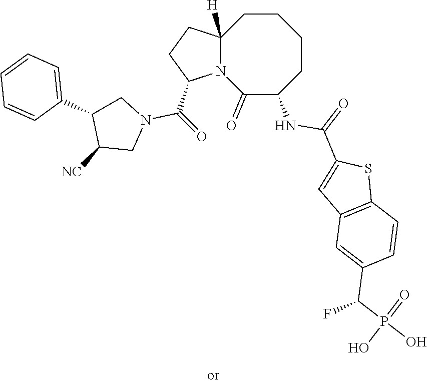 [SMILES: N#C[C@@H]1CN(C(=O)[C@@H]2CC[C@@H]3CCCC[C@H](NC(=O)c4cc5cc([C@@H](F)P(=O)(O)O)ccc5s4)C(=O)N32)C[C@H]1c1ccccc1] | 653.1 [M + H]⁺ | ¹H NMR (400 MHz, methanol-d₄) δ 8.12 (s, 1H), 8.06-8.00 (m, 1H), 7.99-7.91 (m, 1H), 7.63- 7.55 (m, 1H), 7.45- 7.26 (m, 5H), 5.83 (m, 1H), 5.09-4.99 (m, 1H), 4.75-4.64 (m, 1H), 4.58- 4.37 (m, 2H), 4.12- 3.92 (m, 1H), 3.89-3.65 (m, 2H), 3.64-3.50 (m, 2H), 2.40-2.18 (m, 2H), 2.11-1.93 (m, 6H), 1.91- 1.77 (m, 2H), 1.71- 1.58 (m, 2H) |
| | | 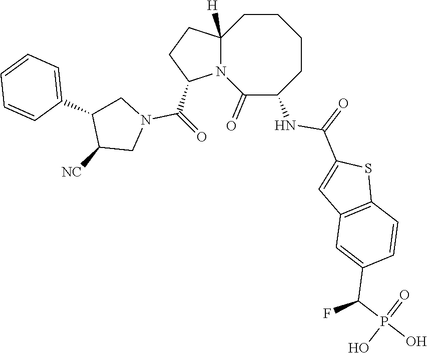 [SMILES: N#C[C@@H]1CN(C(=O)[C@@H]2CC[C@@H]3CCCC[C@H](NC(=O)c4cc5cc([C@H](F)P(=O)(O)O)ccc5s4)C(=O)N32)C[C@H]1c1ccccc1] | | |
| | | SFC peak 2 was used for | | |
| | | biological testing | | |
| 20 | ((2- (((3S,6S,9S,10aR)-9- (azetidin-1-yl)-3-(3- (morpholine-4- carbonyl)azetidine-1- carbonyl)-5- oxodecahydropyrrolo [1,2-a]azocin-6- yl)carbamoyl) benzo[b]thiophen-5- yl)difluoromethyl) phosphonic acid | 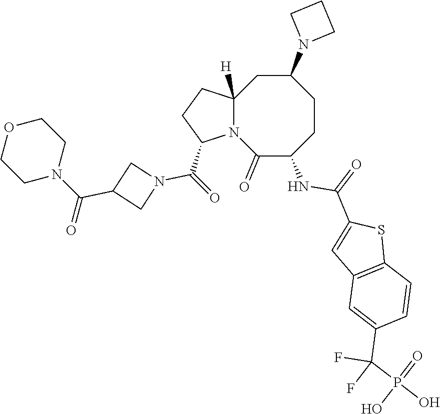 [SMILES: O=C(N[C@H]1CC[C@H](N2CCC2)C[C@H]2CC[C@@H](C(=O)N3CC(C(=O)N4CCOCC4)C3)N2C1=O)c1cc2cc(C(F)(F)P(=O)(O)O)ccc2s1] | 724.1 [M + H]⁺ | ¹H NMR (400 MHz, methanol-d₄) δ 8.22- 8.13 (m, 2H), 8.13-8.04 (m, 1H), 7.78-7.71 (m, 1H), 4.81-4.58 (m, 1H), 4.57-4.37 (m, 1H), 4.36- 4.21 (m, 2H), 4.19- 3.88 (m, 4H), 3.82-3.67 (m, 2H), 3.66-3.54 (m, 6H), 3.53-3.43 (m, 1H), 3.42-3.32 (m, 2H), 3.14- 3.01 (m, 1H), 2.54- 2.37 (m, 1H), 2.35-2.23 (m, 1H), 2.22-2.04 (m, 2H), 2.02-1.79 (m, 2H), 1.78-1.49 (m, 4H), 1.47- 1.36 (m, 1H), 0.66- 0.52 (m, 1H), 0.44-0.30 (m, 1H) |
| 21 | ((2- (((3S,6S,9S,10aR)-9- (azetidin-1-yl)-3- (rel-(trans)-3-cyano-4- phenylpyrrolidine-1- carbonyl)-5- oxodecahydropyrrolo [1,2-a]azocin-6- yl)carbamoyl) benzo[b]thiophen-5- yl)difluoromethyl) phosphonic acid | 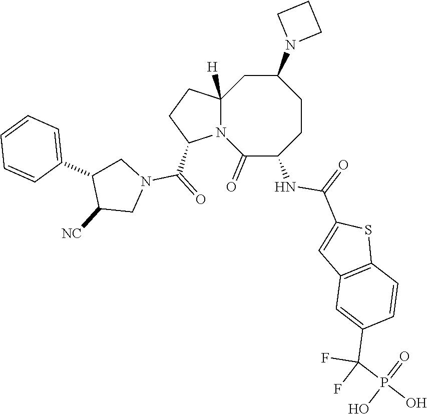 [SMILES: N#C[C@@H]1CN(C(=O)[C@@H]2CC[C@@H]3C[C@@H](N4CCC4)CC[C@H](NC(=O)c4cc5cc(C(F)(F)P(=O)(O)O)ccc5s4)C(=O)N32)C[C@H]1c1ccccc1] | 726.1 [M + H]⁺ | ¹H NMR (400 MHz, methanol-d₄) δ 8.27- 8.12 (m, 2H), 8.10-8.01 (m, 1H), 7.81-7.73 (m, 1H), 7.51-7.21 (m, 5H), 4.63-4.48 (m, 1H), 4.46- 4.30 (m, 1H), 4.16- 3.36 (m, 10H), 3.24-3.11 (m, 1H), 2.51-1.84 (m, 6H), 1.82-1.49 (m, 4H), 1.49-1.36 (m, 1H), 0.69- 0.54 (m, 1H), 0.44- 0.27 (m, 1H) |
| 22 | ((2- (((3S,6S,10aS)-3- ((3S,4R or 3R,4S))- 3-cyano-4- phenylpyrrolidine-1- carbonyl)-5- oxodecahydropyrrolo [1,2-a]azocin-6- yl)carbamoyl)benzo[b] thiophen-5-yl) difluoromethyl) phosphonic acid | 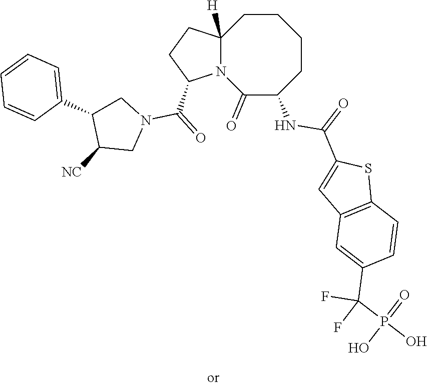 [SMILES: N#C[C@@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1c1ccccc1] | 671.0 [M + H]⁺ | ¹H NMR (400 MHz, methanol-d₄) δ 8.19- 8.13 (m, 2H), 8.02 (t, J = 7.5 Hz, 1H), 7.67 (d, J = 8.8 Hz, 1H), 7.43-7.29 (m, 5H), 5.08-4.99 (m, 1H), 4.75-4.65 (m, 1H), 4.58-4.50 (m, 0.5H), 4.49-4.39 (m, 1.5H), 4.07 (dd, J = 11.5, 8.1 Hz, 0.5H), 3.96 (dd, J = 11.7, 7.6 Hz, 0.5H), 3.85 (t, J = 9.7 Hz, 0.5H), 3.80-3.66 (m, 1.5H), 3.66-3.50 (m, 2H), 2.42-2.17 (m, 2H), 2.13-1.93 (m, 6H), 1.92- 1.75 (m, 2H), 1.75- 1.58 (m, 2H) |
| | | 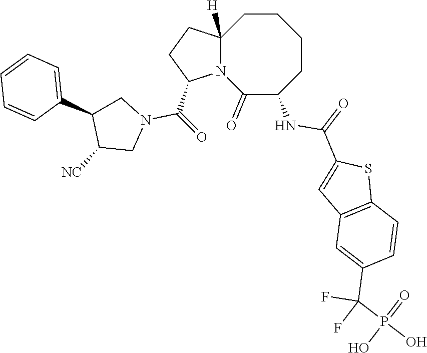 [SMILES: N#C[C@H]1CN(C(=O)[C@@H]2CC[C@@H]3CCCC[C@H](NC(=O)c4cc5cc(C(F)(F)P(=O)(O)O)ccc5s4)C(=O)N32)C[C@@H]1c1ccccc1] | | |
| | | SFC peak 2 was used for | | |
| | | biological testing | | |
| 23 | ((2- (((3S,6S,8R,10aS)-8- (azetidin-1-yl)-3-(rel- trans)-3-cyano-4- phenylpyrrolidine-1- carbonyl)-5- oxodecahydropyrrolo [1,2-a]azocin-6- yl)carbamoyl) benzo[b]thiophen-5- yl)difluoromethyl) phosphonic acid | 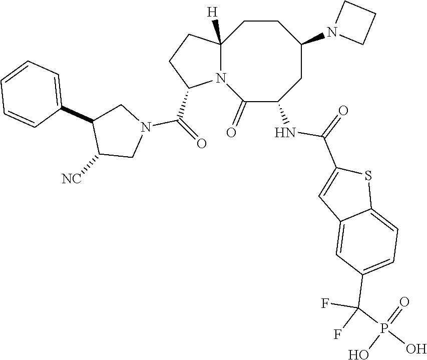 [SMILES: N#C[C@H]1CN(C(=O)[C@@H]2CC[C@@H]3CC[C@@H](N4CCC4)C[C@H](NC(=O)c4cc5cc(C(F)(F)P(=O)(O)O)ccc5s4)C(=O)N32)C[C@@H]1c1ccccc1] | 726.1 [M + H]⁺ | ¹H NMR (400 MHz, methanol-d₄) δ 8.19- 8.14 (m, 1H), 8.13-8.07 (m, 1H), 7.97-7.91 (m, 1H), 7.79-7.70 (m, 1H), 7.44-7.24 (m, 5H), 4.71- 4.56 (m, 1H), 4.45- 3.38 (m, 13H), 2.63-2.46 (m, 1H), 2.45-2.18 (m, 3H), 2.13-1.74 (m, 6H), 1.73-1.56 (m, 1H), 1.17- 1.02 (m, 1H) |
| 24 | ((2- (((3S,6S,10aS)-3- (cinnolin-6-yl (methyl)carbamoyl)-5- oxodecahydropyrrolo [1,2-a]azocin-6- yl)carbamoyl)benzo[b] thiophen-5-yl) difluoromethyl) phosphonic acid | 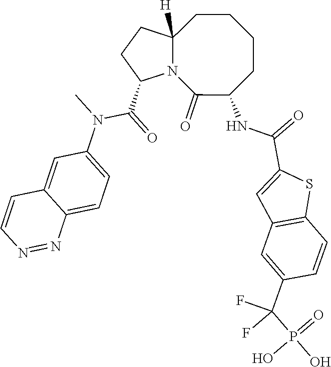 [SMILES: CN(C(=O)[C@@H]1CC[C@@H]2CCCC[C@H](NC(=O)c3cc4cc(C(F)(F)P(=O)(O)O)ccc4s3)C(=O)N21)c1ccc2nnccc2c1] | 658.1 [M + H]⁺ | ¹H NMR (400 MHz, methanol-d₄) δ 9.29 (d, J = 5.9 Hz, 1H), 8.52 (d, J = 9.3 Hz, 1H), 8.30 (d, J = 5.9 Hz, 1H), 8.16-8.13 (m, 1H), 8.13-8.06 (m, 3H), 7.96 (d, J = 8.6 Hz, 1H), 7.67 (d, J = 8.6 Hz, 1H), 5.00 (t, J = 8.7 Hz, 1H), 4.63-4.48 (m, 1H), 4.44-4.31 (m, 1H), 3.45 (s, 3H), 2.22-1.95 (m, 8H), 1.92-1.75 (m, 2H), 1.73-1.57 (m, 2H) |
| 25 | (difluoro(2- (((3′S,6′S,10a′R)-5′- oxo-3′-(3-(pyridin-3- yl)azetidine-1- carbonyl)octahydro- 1′H-spiro [cyclopropane-1,9′- pyrrolo[1,2-a]azocin]- 6′-yl)carbamoyl) benzo[b] thiophen-5-yl)methyl) phosphonic acid | 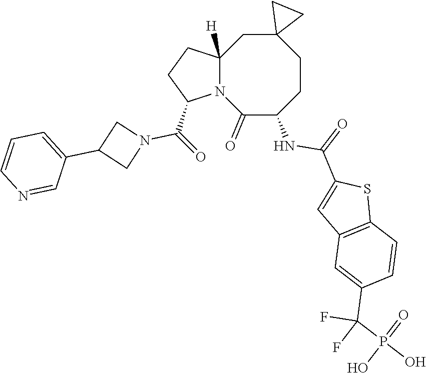 [SMILES: O=C(N[C@H]1CCC2(CC2)C[C@H]2CC[C@@H](C(=O)N3CC(c4cccnc4)C3)N2C1=O)c1cc2cc(C(F)(F)P(=O)(O)O)ccc2s1] | 659.2 [M + H]⁺ | ¹H NMR (400 MHz, methanol-d₄ + 1 drop NaOD) δ 8.59-8.53 (m, 1H), 8.50-8.42 (m, 1H), 8.33-8.26 (m, 1H), 8.19- 8.13 (m, 1H), 8.12- 8.06 (m, 0.4H), 7.98- 7.92 (m, 0.6H), 7.90- 7.82 (m, 2H), 7.53-7.44 (m, 1H), 5.23-5.13 (m, 1H), 5.05-4.97 (m, 1H), 4.75-4.61 (m, 1H), 4.60- 4.27 (m, 3H), 4.17- 3.94 (m, 2H), 2.72-2.51 (m, 2H), 2.35-2.17 (m, 2H), 2.10-1.99 (m, 1H), 1.98-1.85 (m, 2H), 1.84- 1.71 (m, 1H), 1.36- 1.19 (m, 1H), 0.81-0.68 (m, 2H), 0.61-0.47 (m, 2H), 0.43-0.34 (m, 1H) |
| 26 | (difluoro(2- (((3S,6S,10aR,Z)-5- oxo-3-(3- (pyridin-3-yl) azetidine-1-carbonyl)- 1,2,3,5,6,7,8,10a- octahydropyrrolo[1,2- a]azocin-6-yl) carbamoyl)benzo[b] thiophen-5-yl)methyl) phosphonic acid | 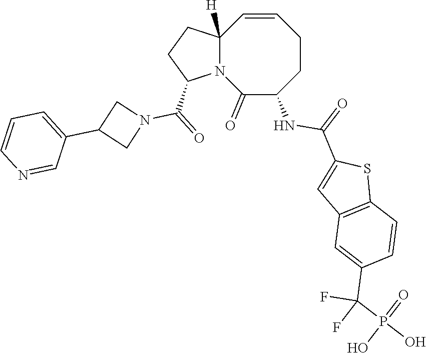 [SMILES: O=C(N[C@H]1CC/C=C\[C@H]2CC[C@@H](C(=O)N3CC(c4cccnc4)C3)N2C1=O)c1cc2cc(C(F)(F)P(=O)(O)O)ccc2s1] | 631.1 [M + H]⁺ | ¹H NMR (400 MHz, methanol-d₄) δ 8.57- 8.51 (m, 1H), 8.49-8.40 (m, 1H), 8.30-8.24 (m, 1H), 8.15 (s, 1H), 8.10- 7.92 (m, 1H), 7.91-7.80 (m, 2H), 7.53-7.41 (m, 1H), 5.88-5.75 (m, 1H), 5.68-5.57 (m, 1H), 5.03- 4.95 (m, 2H), 4.87- 4.79 (m, 1H), 4.74-4.58 (m, 2H), 4.57-4.37 (m, 1H), 4.33-3.96 (m, 2H), 3.24-3.11 (m, 1H), 2.44- 2.31 (m, 1H), 2.25- 1.87 (m, 6H) |
| 27 | ((2- (((3S,6S,10aR,Z)-3- (rel-trans)3- cyano-4- phenylpyrrolidine-1- carbonyl)-5-oxo- 1,2,3,5,6,7,8,10a- octahydropyrrolo[1,2- a]azocin-6- yl)carbamoyl)benzo[b] thiophen-5-yl) difluoromethyl) phosphonic acid | 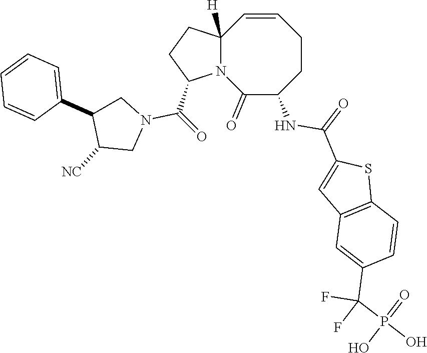 [SMILES: N#C[C@H]1CN(C(=O)[C@@H]2CC[C@@H]3/C=C\CC[C@H](NC(=O)c4cc5cc(C(F)(F)P(=O)(O)O)ccc5s4)C(=O)N32)C[C@@H]1c1ccccc1] | 669.1 [M + H]⁺ | ¹H NMR (400 MHz, methanol-d₄) δ 8.20- 8.12 (m, 2H), 8.06-7.99 (m, 1H), 7.68-7.66 (m, 1H), 7.46-7.28 (m, 5H), 5.85-5.75 (m, 1H), 5.65- 5.62 (m, 1H), 5.01- 4.96 (m, 1H), 4.85-4.77 (m, 2H), 4.52-3.98 (m, 2H), 3.97-3.39 (m, 4H), 3.27-3.17 (m, 1H), 2.43- 2.34 (m, 1H), 2.28- 2.15 (m, 1H), 2.13-1.89 (m, 5H), 1.40-1.09 (m, 1H) |
| 28 | ((7- (((3S,6S,10aS)-3-(rel- trans)-3-cyano-4- phenylpyrrolidine-1- carbonyl)-5- oxodecahydropyrrolo [1,2-a]azocin-6- yl)carbamoyl) naphthalen-2-yl) difluoromethyl) phosphonic acid | 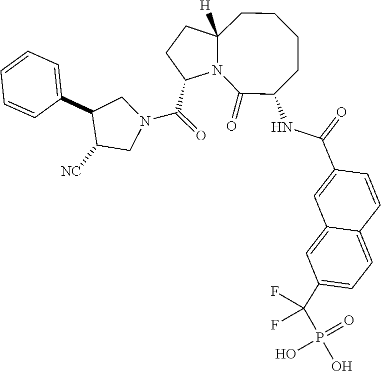 [SMILES: N#C[C@H]1CN(C(=O)[C@@H]2CC[C@@H]3CCCC[C@H](NC(=O)c4ccc5ccc(C(F)(F)P(=O)(O)O)cc5c4)C(=O)N32)C[C@@H]1c1ccccc1] | 665.4 [M + H]⁺ | ¹H NMR (400 MHz, methanol-d₄) δ 8.55- 8.50 (m, 1H), 8.27 (s, 1H), 8.01-7.92 (m, 3H), 7.90-7.83 (m, 1H), 7.49- 7.27 (m, 5H), 5.14- 5.05 (m, 1H), 4.75-4.62 (m, 1H), 4.60-4.41 (m, 2H), 4.17-4.03 (m, 1H), 4.02-3.84 (m, 1H), 3.84- 3.68 (m, 1H), 3.67- 3.45 (m, 2H), 2.42-2.20 (m, 2H), 2.14-1.93 (m, 6H), 1.92-1.77 (m, 2H), 1.76-1.60 (m, 2H) |
| 29 | ((7- (((3S,6S,9aS)-3- ([1,1′-biphenyl]-4- ylcarbamoyl)-5- oxooctahydro-1H- pyrrolo[1,2-a] azepin-6-yl) carbamoyl)naphthalen- 2-yl)difluoromethyl) phosphonic acid | 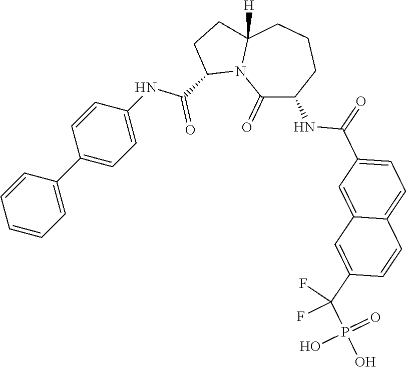 [SMILES: O=C(N[C@H]1CCC[C@H]2CC[C@@H](C(=O)Nc3ccc(-c4ccccc4)cc3)N2C1=O)c1ccc2ccc(C(F)(F)P(=O)(O)O)cc2c1] | 648.2 [M + H]⁺ | ¹H NMR (400 MHz, methanol-d₄) δ 8.53 (s, 1H), 8.24 (s, 1H), 8.07- 7.93 (m, 3H), 7.78 (d, J = 8.8 Hz, 1H), 7.70-7.62 (m, 2H), 7.61-7.52 (m, 4H), 7.41 (t, J = 7.6 Hz, 2H), 7.35-7.26 (m, 1H), 4.86 (s, 1H), 4.74 (dd, J = 8.0, 4.0 Hz, 1H), 4.12 (d, J = 5.6 Hz, 1H), 2.34 (dd, J = 12.4, 5.6 Hz, 1H), 2.26- 2.02 (m, 4H), 2.00- 1.82 (m, 5H) |

Method 2: Representative Procedure for the Synthesis of Phosphonic Acid Analogues by Direct Coupling of Perfluorophenyl or p-Nitrophenyl Activated Linker Esters with Amino Acid Cores

### Synthesis of difluoro(2-(((3S,6S,10aR,Z)-3-(methyl(phenyl)carbamoyl)-5-oxo-1,2,3,5,6,7,10,10a-octahydropyrrolo[1,2-a]azocin-6-yl)carbamoyl)-1H-indol-5-yl)methyl)phosphonic acid (30)

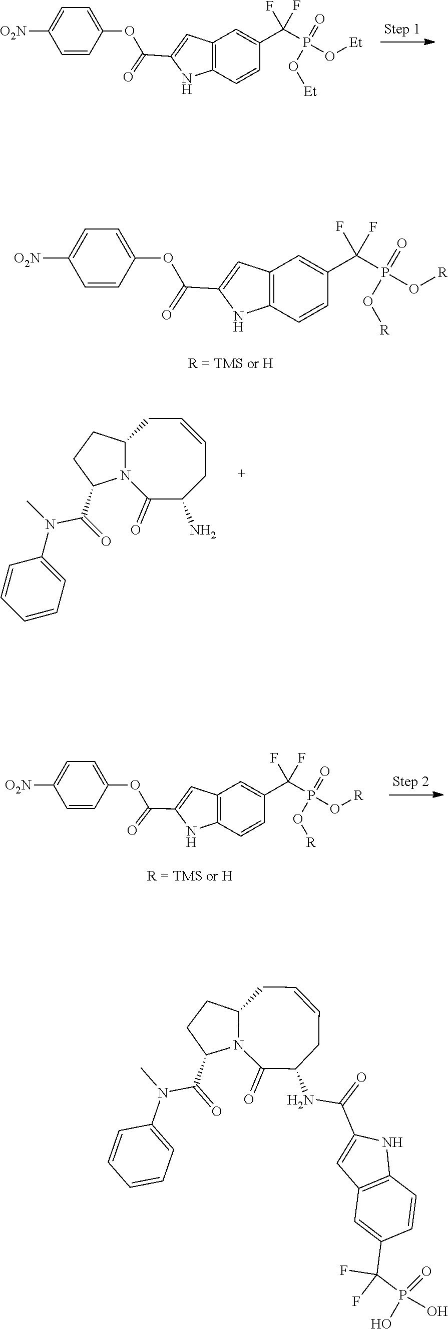

### Step 1: Preparation of 4-nitrophenyl 5-((bis((trimethylsilyl)oxy)phosphoryl)difluoromethyl)-1H-indole-2-carboxylate

To a solution of 4-nitrophenyl 5-((diethoxyphosphoryl)difluoromethyl)-1H-indole-2-carboxylate (70 mg, 149 μmol, 1.0 eq.) in CH₂Cl₂ (4 mL) was added TMSBr (228 mg, 1.49 mmol, 10.0 eq.) dropwise at room temperature. The mixture was stirred at room temperature for 5 hrs. After completion, the reaction was concentrated under reduced pressure to afford 4-nitrophenyl 5-((bis((trimethylsilyl)oxy)phosphoryl)difluoromethyl)-1H-indole-2-carboxylate (77.0 mg, 146 μmol, 99%) as a yellow solid, which was used in next step directly without further purification. LCMS (ESI) m/z=413 [(M−144)+H]⁺. (TMS ester was hydrolysis when LCMS, only detected parent acid mass).

### Step 2: Preparation of (3S,6S,10aR,Z)-6-amino-N-methyl-5-oxo-N-phenyl-1,2,3,5,6,7,10,10a-octahydropyrrolo[1,2-a]azocine-3-carboxamide

(3S,6S,10aR,Z)-6-amino-N-methyl-5-oxo-N-phenyl-1,2,3,5,6,7,10,10a-octahydropyrrolo[1,2-a]azocine-3-carboxamide was synthesized starting from (3S,6S,10aR,Z)-6-((tert-butoxycarbonyl)amino)-5-oxo-1,2,3,5,6,7,10,10a-octahydropyrrolo[1,2-a]azocine-3-carboxylic acid and using similar protocol described above for the preparation of (3S,6S,10aS)-6-amino-N-methyl-5-oxo-N-phenyldecahydropyrrolo[1,2-a]azocine-3-carboxamide.

### Step 1: Preparation of Difluoro (2-(((3S,6S,10aR,Z)-3-(methyl(phenyl)carbamoyl)-5-oxo-1,2,3,5,6,7,10,10a-octahydropyrrolo[1,2-a]azocin-6-yl)carbamoyl)-1H-indol-5-yl)methyl)phosphonic acid (30)

To a solution of (3S,6S,10aR,Z)-6-amino-N-methyl-5-oxo-N-phenyl-1,2,3,5,6,7,10,10a-octahydropyrrolo[1,2-a]azocine-3-carboxamide (137 mg, 440 μmol, 1.1 eq) [previously synthesized using similar protocol detailed in step 1 and step 2 of Method 1 for the preparation of difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonic acid (1)] and DMAP (146 mg, 1.20 mmol, 3 eq) in DMF (3 mL) was added 4-nitrophenyl 5-({bis[(trimethylsilyl)oxy]phosphoryl}difluoromethyl)-1H-indole-2-carboxylate (223 mg, 400 μmol, 1 eq). The resulting mixture was stirred 40° C. for 12 h. The reaction mixture was cooled to ambient temperatures and H₂O (10 mL) was subsequently introduced. The biphasic mixture was extracted with CH₂Cl₂ (10 mL×3). The combined organic layers were washed with brine (2×20 mL), dried over with anhydrous Na₂SO₄, and concentrated in vacuo. The residue was purified by reverse phase HPLC to afford (difluoro(2-(((3S,6S,10aR,Z)-3-(methyl(phenyl)carbamoyl)-5-oxo-1,2,3,5,6,7,10,10a-octahydropyrrolo[1,2-a]azocin-6-yl)carbamoyl)-1H-indol-5-yl)methyl)phosphonic acid (30) (77 mg, 132 μmol, 33% yield) as a white solid. LCMS (ESI) m/z=587 [M+H]⁺; ¹H NMR (400 MHz, DMSO-d₆) δ 11.80 (s, 1H), 8.55 (d, J=7.6 Hz, 1H), 7.83 (s, 1H), 7.52-7.34 (m, 8H), 5.88-5.73 (m, 2H), 5.03-4.93 (m, 1H), 4.26-4.11 (m, 1H), 3.18-3.11 (m, 4H), 2.67-2.56 (m, 2H), 2.54-2.40 (m, 2H), 1.97-1.73 (m, 4H).

The following compounds in Table 28 were prepared according to the representative procedure described above for the synthesis of (difluoro(2-(((3S,6S,10aR,Z)-3-(methyl(phenyl)carbamoyl)-5-oxo-1,2,3,5,6,7,10,10a-octahydropyrrolo[1,2-a]azocin-6-yl)carbamoyl)-1H-indol-5-yl)methyl)phosphonic acid and utilizing appropriate starting materials and modifications.

TABLE 28

| Com- | | | | |
|---|---|---|---|---|
| pound | Name | Structure | LCMS | NMR |
| 31 | ((2- (((3S,6S,9aS)- 3-(((1H- pyrrolo[2,3- b]pyridin-3- yl)methyl) carbamoyl)-5- oxooctahydro- 1H-pyrrolo[1,2-a] azepin-6-yl) carbamoyl)benzo [b]thiophen-5- yl)difluoromethyl) phosphonic acid | 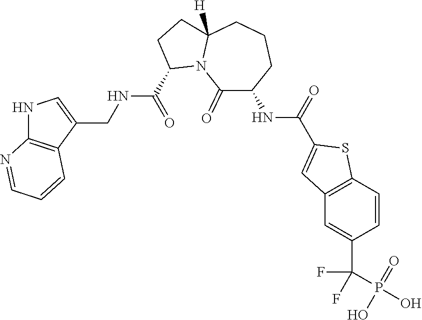 [SMILES: O=C(N[C@H]1CCC[C@H]2CC[C@@H](C(=O)NCc3c[nH]c4ncccc34)N2C1=O)c1cc2cc(C(F)(F)P(=O)(O)O)ccc2s1] | 632.1 [M + H]⁺ | ¹H NMR (400 MHz, methanol-d₄) δ 8.27- 8.14 (m, 2H), 8.10-8.05 (m, 2H), 7.91 (d, J = 8.5 Hz, 1H), 7.75 (d, J = 8.6 Hz, 1H), 7.38 (s, 1H), 7.12-7.08 (m, 1H), 4.71 (d, J = 11.2 Hz, 1H), 4.61- 4.50 (m, 3H), 4.06- 3.97 (m, 1H), 2.31-2.21 (m, 1H), 2.07-1.97 (m, 4H), 1.93-1.81 (m, 3H), 1.80-1.67 (m, 2H) |
| 32 | (difluoro(2- (((3S,6S,9aS)- 3-(3-((S- methyl- sulfonimidoyl) methyl) azetidine-1- carbonyl)-5- oxooctahydro- 1H-pyrrolo[1,2- a]azepin-6- yl)carbamoyl) benzo[b] thiophen-5- yl)methyl) phosphonic acid | 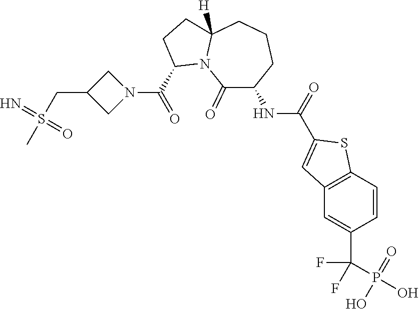 [SMILES: CS(=N)(=O)CC1CN(C(=O)[C@@H]2CC[C@@H]3CCC[C@H](NC(=O)c4cc5cc(C(F)(F)P(=O)(O)O)ccc5s4)C(=O)N32)C1] | 633.3 [M + H]⁺ | ¹H NMR (400 MHz, methanol-d₄) δ 8.21- 8.07 (m, 2H), 7.98 (d, J = 8.4 Hz, 1H), 7.74-7.66 (m, 1H), 4.49-4.36 (m, 2H), 4.45-4.35 (m, 2H), 4.24-4.11 (m, 3H), 4.09- 4.03 (m, 1H), 3.97- 3.85 (m, 1H), 3.64-3.54 (m, 3H), 2.29 (m, 1H), 2.12-1.99 (m, 3H), 1.98- 1.74 (m, 7H) |
| 33 | (difluoro(2- (((3S,6S,9S)- 5-oxo-3-(3- (pyridin-3- yl)azetidine- 1- carbonyl) octahydro-1H- pyrrolo[1,2-a] azepin-6- yl)carbamoyl) benzo[b] thiophen-5- yl)methyl) phosphonic acid | 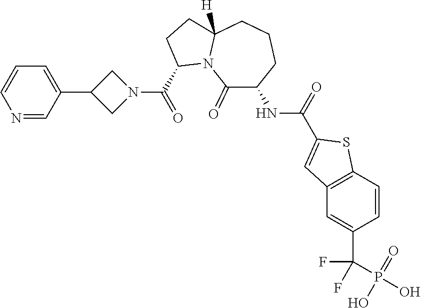 [SMILES: O=C(N[C@H]1CCC[C@H]2CC[C@@H](C(=O)N3CC(c4cccnc4)C3)N2C1=O)c1cc2cc(C(F)(F)P(=O)(O)O)ccc2s1] | 619.0 [M + H]⁺ | ¹H NMR (400 MHz, methanol-d₄) δ 8.54 (d, J = 1.6 Hz, 1H), 8.49-8.40 (m, 1H), 8.22 (s, 1H), 8.16-7.89 (m, 3H), 7.78 (d, J = 8.4 Hz, 1H), 7.55- 7.39 (m, 1H), 4.80-4.65 (m, 2H), 4.63-4.47 (m, 2H), 4.45-4.24 (m, 1H), 4.15-3.92 (m, 3H), 2.31 (d, J = 6.4 Hz, 1H), 2.22- 1.77 (m, 9H), 1.38-1.29 (m, 3H) |
| 34 | (difluoro(2- (((3S,6S,9aS)- 3-(3- morpholino- azetidine-1- carbonyl)-5- oxooctahydro- 1H- pyrrolo[1,2-a] azepin-6- yl)carbamoyl) benzo[b] thiophen-5- yl)methyl) phosphonic acid | 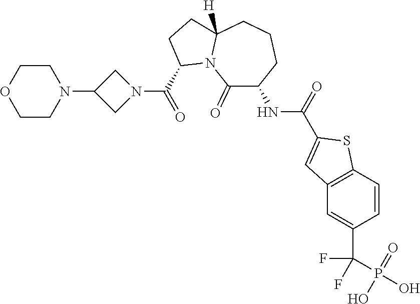 [SMILES: O=C(N[C@H]1CCC[C@H]2CC[C@@H](C(=O)N3CC(N4CCOCC4)C3)N2C1=O)c1cc2cc(C(F)(F)P(=O)(O)O)ccc2s1] | 627.3 [M + H]⁺ | ¹H NMR (400 MHz, DMSO-d₆) δ 8.74 (dd, J = 7.6, 14.4 Hz, 1H), 8.28 (d, J = 6.4 Hz, 1H), 8.12 (d, J = 8.4 Hz, 1H), 8.07 (s, 1H), 7.59 (d, J = 8.4 Hz, 1H), 4.63 (d, J = 5.2 Hz, 1H), 4.55-4.48 (m, 1H), 4.40 (dd, J = 8.4, 4.8 Hz, 1H), 4.31 (d, J = 3.2 Hz, 1H), 3.97 (dd, J = 4.8, 10.0 Hz, 4H), 3.75-3.68 (m, 4H), 2.92-2.81 (m, 2H), 2.68 (d, J = 11.2 Hz, 2H), 2.23-2.17 (m, 1H), 2.01-1.93 (m, 2H), 1.91- 1.82 (m, 2H), 1.78 (d, J = 4.0 Hz, 2H), 1.64 (s, 3H) |
| 35 | ((4-((E)-3- (((3S,6S,9S)- 3-([1,1′- biphenyl]-4- ylcarbamoyl)- 5- oxooctahydro- 1H-pyrrolo[1,2- a]azepin-6- yl)amino)-3- oxoprop-1- en-1- yl)phenyl) difluoromethyl) phosphonic acid | 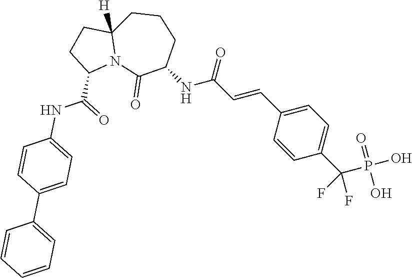 [SMILES: O=C(/C=C/c1ccc(C(F)(F)P(=O)(O)O)cc1)N[C@H]1CCC[C@H]2CC[C@@H](C(=O)Nc3ccc(-c4ccccc4)cc3)N2C1=O] | 624.5 [M + H]⁺ | ¹H NMR (400 MHz, methanol-d₄) δ 7.70- 7.51 (m, 11H), 7.41 (t, J = 7.6 Hz, 2H), 7.34-7.24 (m, 1H), 6.80 (d, J = 15.6 Hz, 1H), 4.75-4.65 (m, 2H), 4.17-3.95 (m, 1H), 2.40-2.26 (m, 1H), 2.23- 2.02 (m, 3H), 1.98- 1.73 (m, 6H) |
| 36 | ((2- (((3S,6S,10aS)- 3-(3- benzylazetidine- 1-carbonyl)-5- oxodecahydro- pyrrolo[1,2-a] azocin-6- yl)carbamoyl) benzo[b] thiophen-5-yl) difluoromethyl) phosphonic acid | 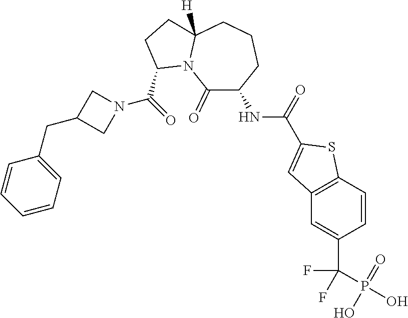 [SMILES: O=C(N[C@H]1CCC[C@H]2CC[C@@H](C(=O)N3CC(Cc4ccccc4)C3)N2C1=O)c1cc2cc(C(F)(F)P(=O)(O)O)ccc2s1] | 646.4 [M + H]⁺ | ¹H NMR (400 MHz, methanol-d₄) δ 8.21- 8.10 (m, 2H), 7.98-7.91 (m, 1H), 7.78-7.70 (m, 1H), 7.30-7.12 (m, 5H), 5.07-4.98 (m, 1H), 4.82 (s, 1H), 4.46-4.35 (m, 2H), 4.26 (d, J = 5.6 Hz, 1H), 4.02-3.90 (m, 1H), 3.82-3.62 (m, 1H), 3.03- 2.88 (m, 3H), 2.29- 2.13 (m, 2H), 2.06-1.91 (m, 6H), 1.88-1.73 (m, 2H), 1.72-1.54 (m, 2H) |
| 37 | ((2- (((3S,6S,10aS)- 3-(((1H- pyrazolo[3,4- b]pyridin-3- yl)methyl) carbamoyl)-5- oxodecahydro- pyrrolo[1,2- a]azocin-6- yl)carbamoyyl) benzo[b] thiophen-5- yl)difluoro- methyl) phosphonic acid | 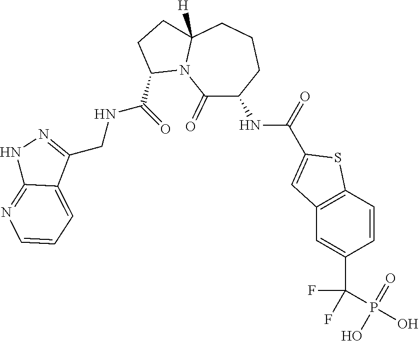 [SMILES: O=C(N[C@H]1CCC[C@H]2CC[C@@H](C(=O)NCc3n[nH]c4ncccc34)N2C1=O)c1cc2cc(C(F)(F)P(=O)(O)O)ccc2s1] | 647.1 [M + H]⁺ | ¹H NMR (400 MHz, methanol-d₄) δ 8.50- 8.43 (m, 1H), 8.40-8.34 (m, 1H), 8.16 (s, 1H), 8.10 (s, 1H), 7.91 (d, J = 8.8 Hz, 1H), 7.73 (d, J = 8.8 Hz, 1H), 7.23-7.15 (m, 1H), 5.08-5.01 (m, 1H), 4.74 (m, 2H), 4.49- 4.38 (m, 2H), 2.26-2.15 (m, 2H), 2.08-1.93 (m, 6H), 1.86-1.76 (m, 2H), 1.73-1.58 (m, 2H) |
| 38 | (difluoro(2- (((3S,6S,10aS)- 3-(3-(1- methyl-1H- benzo[d]imidazol-2- yl)azetidine-1- carbonyl)-5- oxodecahydropyrrolo [1,2-a]azocin-6- yl)carbamoyl) benzo[b]thiophen- 5-yl)methyl) phosphonic acid | 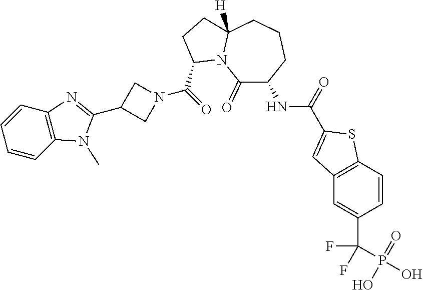 | 686.2 [M + H]⁺ | ¹H NMR (400 MHz, methanol-d₄) δ 8.21 (s, 1H), 8.16-8.07 (m, 1H), 7.94-7.83 (m, 1H), 7.79- 7.71 (m, 1H), 7.67- 7.56 (m, 1H), 7.50-7.37 (m, 1H), 7.32-7.18 (m, 2H), 5.05-4.98 (m, 1H), 4.79-4.70 (m, 1H), 4.65- 4.52 (m, 2H), 4.52- 4.39 (m, 2H), 4.38-4.27 (m, 2H), 3.77-3.65 (m, 3H), 2.35-2.17 (m, 2H), 2.11-1.89 (m, 6H), 1.88- 1.74 (m, 2H), 1.73- 1.57 (m, 2H) |
| 39 | (difluoro(2- (((3S,6S,10aS)- 5-oxo-3-(3- phenoxyazetidine-1- carbonyl) decahydropyrrolo [1,2-a]azocin-6- yl)carbamoyl) benzo[b]thiophen- 5-yl)methyl) phosphonic acid | 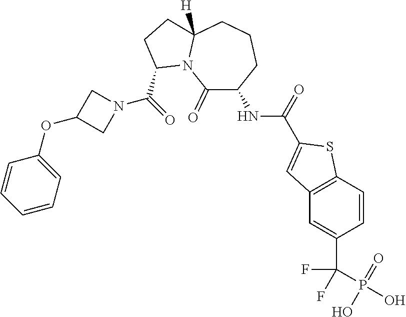 [SMILES: O=C(N[C@H]1CCC[C@H]2CC[C@@H](C(=O)N3CC(Oc4ccccc4)C3)N2C1=O)c1cc2cc(C(F)(F)P(=O)(O)O)ccc2s1] | 648.1 [M + H]⁺ | ¹H NMR (400 MHz, methanol-d₄) δ 8.21- 8.07 (m, 2H), 7.95-7.90 (m, 1H), 7.79-7.69 (m, 1H), 7.32-7.21 (m, 2H), 7.00-6.86 (m, 1H), 6.82- 7.79 (m, 2H), 5.10- 4.88 (m, 3H), 4.71-4.52 (m, 1H), 4.50-4.39 (m, 2H), 4.37-4.18 (m, 1H), 4.06-3.88 (m, 1H), 2.32- 2.16 (m, 2H), 2.08- 1.90 (m, 6H), 1.89-1.73 (m, 2H), 1.72-1.55 (m, 2H) |
| 40 | (difluoro(2- (((3S,6S,10aS)- 6-methyl-3-(3- (morpholine-4- carbonyl)azetidine- 1-carbonyl)-5- oxodecahydropyrrolo [1,2-a]azocin-6- yl)carbamoyl) benzo[b]thiophen- 5-yl)methyl) phosphonic acid | 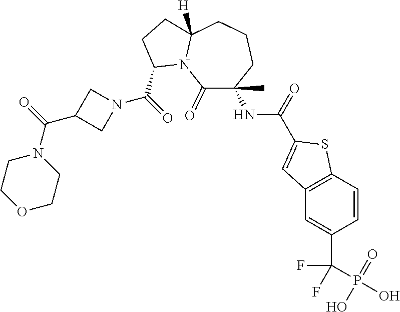 | 683.2 [M + H]⁺ | ¹H NMR (400 MHz, methanol-d₄) δ 8.34- 8.13 (m, 1H), 8.05-7.91 (m, 1H), 7.75 (d, J = 8.8 Hz, 1H), 7.63-7.34 (m, 1H), 4.74-4.65 (m, 1H), 4.54-4.28 (m, 2H), 4.23- 4.02 (m, 1H), 3.97- 3.57 (m, 6H), 3.46-3.37 (m, 1H), 3.24-3.15 (m, 1H), 3.09-2.94 (m, 1H), 2.48-2.29 (m, 1H), 2.25- 2.09 (m, 1H), 2.07- 1.67 (m, 6H), 1.65-1.48 (m, 2H), 1.46-1.29 (m, 10H) |
| 41 | ((7- (((3S,6S,10aS)-3- ((3S,4′S)- 4′-cyano- 2-oxospiro [indoline- 3,3′-pyrrolidine]- 1′-carbonyl)-5- oxodecahydro- pyrrolo[1,2-a] azocin-6-yl) carbamoyl) naphthalen-2-yl) difluoromethyl) phosphonic acid or ((7- (((3S,6S,10aS)- 3-((3R,4′R)-4′- cyano-2-oxospiro [indoline-3,3′- pyrrolidine]-1′- carbonyl)-5-oxo- decahydropyrrolo [1,2-a]azocin-6- yl)carbamoyl) naphthalen-2- yl)difluoromethyl) phosphonic acid or | 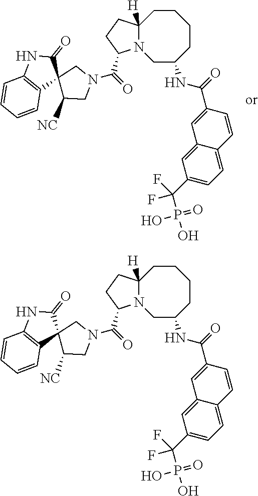 [SMILES: N#C[C@@H]1CN(C(=O)[C@@H]2CC[C@@H]3CCCC[C@H](NC(=O)c4ccc5ccc(C(F)(F)P(=O)(O)O)cc5c4)CN32)C[C@]12C(=O)Nc1ccccc12 ; N#C[C@H]1CN(C(=O)[C@@H]2CC[C@@H]3CCCC[C@H](NC(=O)c4ccc5ccc(C(F)(F)P(=O)(O)O)cc5c4)CN32)C[C@@]12C(=O)Nc1ccccc12] | 706.0 [M + H]⁺ | ¹H NMR (400 MHz, methanol-d₄) δ 8.55- 8.44 (m, 1H), 8.27 (d, J = 13.6 Hz, 1H), 8.01-7.84 (m, 4H), 7.49-7.35 (m, 1H), 7.35-7.28 (m, 1H), 7.17-7.07 (m, 1H), 7.04- 6.94 (m, 1H), 5.17- 5.06 (m, 1H), 4.79 (m, 1H), 4.71-4.61 (m, 1H), 4.56-4.41 (m, 1H), 4.32- 4.22 (m, 1H), 4.21- 3.98 (m, 1H), 3.90 (s, 1H), 3.84-3.76 (m, 1H), 2.57-2.20 (m, 2H), 2.18- 1.97 (m, 6H), 1.92- 1.78 (m, 2H), 1.77-1.61 (m, 2H) |
| | ((7- (((3S,6S,10aS)- 3-((3R,4′S)-4′- cyano-2-oxospiro [indoline-3,3′- pyrrolidine]-1′- carbonyl)-5- oxodecahydro- pyrrolo[1,2-a] azocin-6-yl) carbamoyl) naphthalen-2-yl) difluoromethyl) phosphonic acid or ((7- (((3S,6S,10aS)- 3-((3S,4′R)- 4′-cyano- 2-oxospiro[indoline- 3,3′-pyrrolidine]- 1′-carbonyl)-5- oxodecahydropyrrolo [1,2-a]azocin-6- yl)carbamoyl) naphthalen-2- yl)difluoromethyl) phosphonic acid | 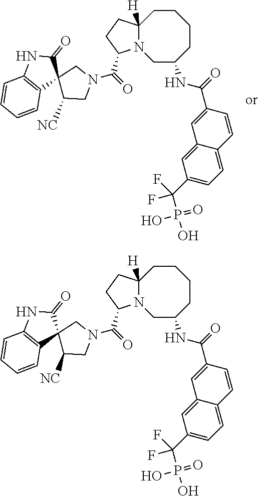 [SMILES: N#C[C@H]1CN(C(=O)[C@@H]2CC[C@@H]3CCCC[C@H](NC(=O)c4ccc5ccc(C(F)(F)P(=O)(O)O)cc5c4)CN32)C[C@]12C(=O)Nc1ccccc12 ; N#C[C@@H]1CN(C(=O)[C@@H]2CC[C@@H]3CCCC[C@H](NC(=O)c4ccc5ccc(C(F)(F)P(=O)(O)O)cc5c4)CN32)C[C@@]12C(=O)Nc1ccccc12] | | |
| | | SFC Peak 2 was used for | | |
| | | biological testing | | |
| 42 | (difluoro(2- (((3S,6S,10aS)-5- oxo-3- (6-(2- phenylacetyl)-2,6- diazaspiro[3.3] heptane-2- carbonyl) decahydropyrrolo [1,2-a]azocin-6- yl)carbamoyl) benzo[b]thiophen- 5-yl)methyl) phosphonic acid | 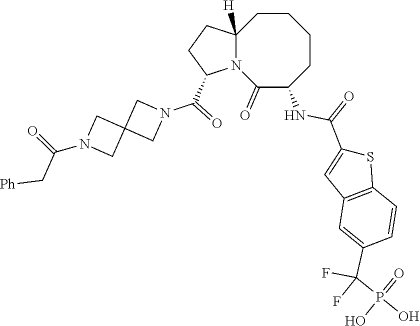 [SMILES: O=C(N[C@H]1CCCC[C@H]2CC[C@@H](C(=O)N3CC4(CN(C(=O)Cc5ccccc5)C4)C3)N2C1=O)c1cc2cc(C(F)(F)P(=O)(O)O)ccc2s1] | 715.2 [M + H]⁺ | ¹H NMR (400 MHz, methanol-d₄) δ 8.15 (d, J = 2.0 Hz, 2H), 8.02 (d, J = 8.4 Hz, 1H), 7.67 (d, J = 8.4 Hz, 1H), 7.35-7.13 (m, 5H), 5.04-4.93 (m, 1H), 4.69-4.59 (m, 1H), 4.38 (d, J = 4.8 Hz, 3H), 4.36-4.32 (m, 2H), 4.17 (s, 1H), 4.14 (d, J = 6.0 Hz, 2H), 4.07 (d, J = 10.4 Hz, 1H), 3.47 (d, J = 8.0 Hz, 2H), 2.25-2.16 (m, 2H), 2.04-1.90 (m, 6H), 1.87-1.76 (m, 2H), 1.72- 1.54 (m, 2H) |
| 43 | ((2- (((3S,6S,9aS)- 3-(3-(1H- benzo[d]imidazol- 2- yl)azetidine-1- carbonyl)-5- oxooctahydro-1H- pyrrolo[1,2-a] azepin-6-yl) carbamoyl) benzo[b]thiophen- 5-yl) difluoromethyl) phosphonic acid | 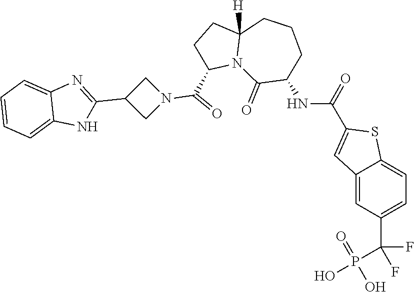 [SMILES: O=C(N[C@H]1CCC[C@H]2CC[C@@H](C(=O)N3CC(c4nc5ccccc5[nH]4)C3)N2C1=O)c1cc2cc(C(F)(F)P(=O)(O)O)ccc2s1] | 658.2 [M + H]⁺ | ¹H NMR (600 MHz, DMSO-d₆) δ 8.78 (d, J = 8.1 Hz, 1H), 8.72 (app. d, J = 21.1 Hz, 1H), 8.16- 7.99 (m, 2H), 7.70-7.55 (m, 2H), 7.50 (d, J = 7.3 Hz, 1H), 7.24 (dd, J = 6.4, 3.1 Hz, 1H), 7.13 (t, J = 4.3 Hz, 1H), 4.79 (s, 1H), 4.64 (m, 2H), 4.45 (m, 2H), 4.32-4.25 (m, 1H), 4.25-4.13 (m, 3H), 4.00 (s, 2H), 2.21 (s, 1H), 1.97 (m, 2H), 1.79 (m, 7H) |
| 44 | (difluoro(2- (((3S,6S,10aS)-3- ((6-methoxypyridin- 3- yl)(methyl) carbamoyl)-5- oxodecahydro- pyrrolo[1,2- a]azocin-6- yl)carbamoyl) benzo[b]thiophen- 5-yl)methyl) phosphonic acid | 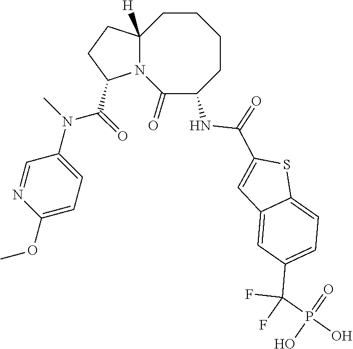 [SMILES: COc1ccc(N(C)C(=O)[C@@H]2CC[C@@H]3CCCC[C@H](NC(=O)c4cc5cc(C(F)(F)P(=O)(O)O)ccc5s4)C(=O)N32)cn1] | 637.2 [M + H]⁺ | ¹H NMR (600 MHz, DMSO-d₆) δ 8.84 (d, J = 7.0 Hz, 1H), 8.32 (s, 1H), 8.22 (d, J = 2.7 Hz, 1H), 8.11 (d, J = 8.5 Hz, 1H), 8.06 (s, 1H), 7.74 (dd, J = 8.7, 2.7 Hz, 1H), 7.57 (d, J = 8.7 Hz, 1H), 6.88 (d, J = 8.7 Hz, 1H), 4.96-4.87 (m, 1H), 4.26-4.08 (m, 3H), 3.83 (s, 4H), 2.04 (s, 1H), 1.85 (m, 7H), 1.65 (m, 2H), 1.53 (m, 2H) |
| 45 | (difluoro(2- (((3S,6S,9aS)-3- (3-hydroxy-3- (pyridin-2-yl) azetidine-1- carbonyl)-5- oxooctahydro-1H- pyrrolo[1,2-a] azepin-6-yl) carbamoyl) benzo[b]thiophen- 5-yl)methyl) phosphonic acid | 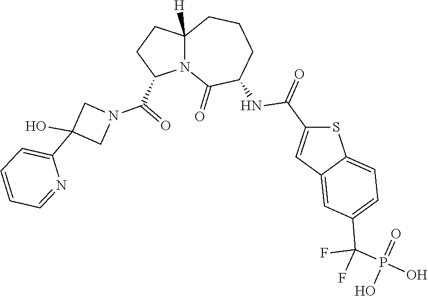 [SMILES: O=C(N[C@H]1CCC[C@H]2CC[C@@H](C(=O)N3CC(O)(c4ccccn4)C3)N2C1=O)c1cc2cc(C(F)(F)P(=O)(O)O)ccc2s1] | 635.2 [M + H]⁺ | ¹H NMR (600 MHz, DMSO-d₆) δ 8.76 (d, J = 7.6 Hz, 1H), 8.64-8.53 (m, 1H), 8.29 (dd, J = 5.8, 2.5 Hz, 1H), 8.12 (dd, J = 8.4, 3.0 Hz, 1H), 8.07 (d, J = 6.6 Hz, 1H), 7.82 (q, J = 7.5 Hz, 1H), 7.63 (d, J = 7.8 Hz, 1H), 7.57 (d, J = 8.6 Hz, 1H), 7.32 (dd, J = 9.7, 5.2 Hz, 1H), 4.79- 4.60 (m, 2H), 4.59-4.46 (m, 2H), 4.35 (m, 2H), 4.18 (m, 2H), 3.96 (s, 2H), 3.88 (d, J = 10.2 Hz, 1H), 2.24-2.15 (m, 1H), 2.07-1.93 (m, 2H), 1.88- 1.68 (m, 6H) |
| 46 | (difluoro(2- (((3S,6S,9aS)-5- oxo-3-(3- (pyrimidin-2- yl)azetidine-1- carbonyl)octahydro- 1H-pyrrolo[1,2- a]azepin-6- yl)carbamoyl) benzo[b] thiophen-5- yl)methyl) phosphonic acid | 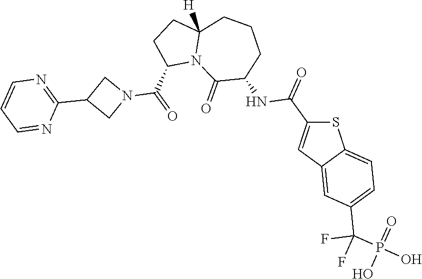 [SMILES: O=C(N[C@H]1CCC[C@H]2CC[C@@H](C(=O)N3CC(c4ncccn4)C3)N2C1=O)c1cc2cc(C(F)(F)P(=O)(O)O)ccc2s1] | 620.2 [M + H]⁺ | ¹H NMR (600 MHz, DMSO-d₆) δ 8.78 (m, 2H), 8.28 (app. d, J = 20.5 Hz, 1H), 8.22-8.07 (m, 1H), 8.05 (d, J = 13.5 Hz, 1H), 7.56 (dd, J = 9.0, 4.1 Hz, 1H), 7.39 (dt, J = 17.1, 4.9 Hz, 1H), 4.80- 4.36 (m, 4H), 4.29-3.94 (m, 4H), 3.04 (pd, J =7.2, 3.6 Hz, 1H), 2.18 (dt, J = 13.7, 6.6 Hz, 1H), 2.10- 1.89 (m, 2H), 1.85-1.64 (m, 5H), 1.13 (app. t, J = 7.3 Hz, 2H) |
| 47 | (difluoro(2- (((3S,6S,10aS)- 3-((2-(2- methoxypyridin- 4-yl)ethyl)(methyl) carbamoyl)-5- oxodecahydropyrrolo [1,2-a]azocin-6- yl)carbamoyl) benzo[b]thiophen- 5-yl)methyl) phosphonic acid | 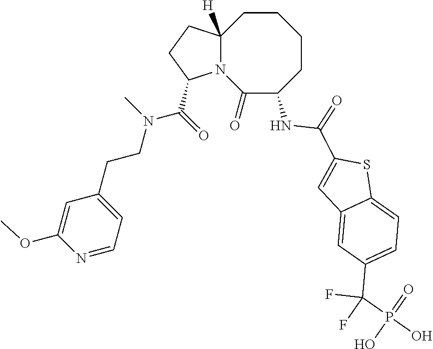 [SMILES: COc1cc(CCN(C)C(=O)[C@@H]2CC[C@@H]3CCCC[C@H](NC(=O)c4cc5cc(C(F)(F)P(=O)(O)O)ccc5s4)C(=O)N32)ccn1] | 665.2 [M + H]⁺ | ¹H NMR (600 MHz, DMSO-d₆) δ 8.85 (d, J = 6.6 Hz, 1H), 8.31 (s, 1H), 8.12 (d, J = 8.6 Hz, 1H), 8.04 (t, J = 4.5 Hz, 2H), 7.57 (d, J = 8.6 Hz, 1H), 6.90 (dd, J = 23.6, 5.3 Hz, 1H), 6.72 (d, J = 28.9 Hz, 1H), 4.96 (app. s, 1H), 4.71 (m, 1H), 4.26 (m, 1H), 3.64 (s, 2H), 3.41- 3.36 (m, 1H), 3.00 (s, 2H), 2.91 (s, 1H), 2.72 (m, 3H), 2.27-1.77 (m, 7H), 1.74-1.39 (m, 5H) |
| 48 | (difluoro(2- (((3S,6S,10aS)-5- oxo-3-(5-oxo- 1,2,3,4,5,6- hexahydro-2,6- naphthyridine-2- carbonyl) decahydropyrrolo [1,2-a]azocin-6- yl)carbamoyl) benzo[b]thiophen- 5-yl)methyl) phosphonic acid | 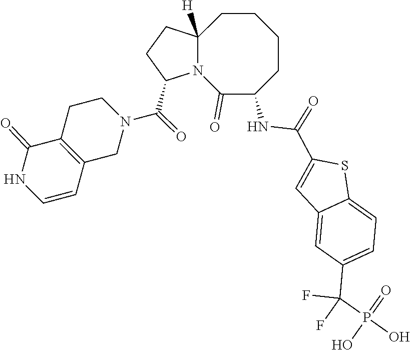 [SMILES: O=C(N[C@H]1CCCC[C@H]2CC[C@@H](C(=O)N3CCc4c(cc[nH]c4=O)C3)N2C1=O)c1cc2cc(C(F)(F)P(=O)(O)O)ccc2s1] | 649.0 [M + H]⁺ | ¹H NMR (600 MHz, DMSO-d₆) δ 11.41 (s, 1H), 8.83 (dd, J = 16.6, 7.0 Hz, 1H), 8.29 (s, 1H), 8.11 (d, J = 8.7 Hz, 1H), 8.04 (s, 1H), 7.56 (d, J = 8.7 Hz, 1H), 7.19 (d, J = 6.7 Hz, 1H), 6.01 (t, J = 8.4 Hz, 1H), 5.01-4.82 (m, 2H), 4.76-4.49 (m, 2H), 4.27 (m, 2H), 3.89 (m, 1H), 3.63 (s, 4H), 2.30 (m, 2H), 2.08 (m, 2H), 1.77 (m, 5H), 1.56 (m, 3H) |
| 49 | (difluoro(2- (((3S,6S,10aS)- 5-oxo-3- (((2-oxo-1,2- dihydropyridin-4- yl)methyl) carbamoyl)deca- hydropyrrolo[1,2- a]azocin-6- yl)carbamoyl) benzo[b]thiophen- 5-yl)methyl) phosphonic acid | 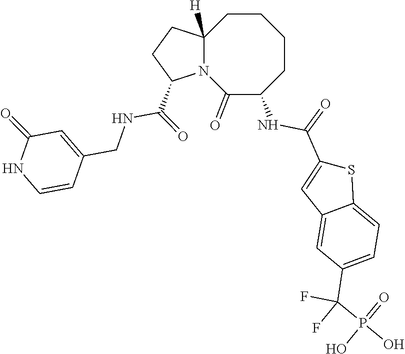 [SMILES: O=C(N[C@H]1CCCC[C@H]2CC[C@@H](C(=O)NCc3cc[nH]c(=O)c3)N2C1=O)c1cc2cc(C(F)(F)P(=O)(O)O)ccc2s1] | 623.2 [M + H]⁺ | ¹H NMR (600 MHz, DMSO-d₆) δ 11.38 (s, 1H), 8.93 (d, J = 6.8 Hz, 1H), 8.49 (t, J = 6.1 Hz, 1H), 8.32 (s, 1H), 8.13 (d, J = 8.4 Hz, 1H), 8.06 (s, 1H), 7.57 (d, J = 8.6 Hz, 1H), 7.27 (d, J = 6.7 Hz, 1H), 6.18 (s, 1H), 6.06 (d, J = 6.8 Hz, 1H), 4.94 (dt, J = 11.6, 5.9 Hz, 1H), 4.35 (t, J = 8.5 Hz, 1H), 4.28 (t, J = 9.8 Hz, 1H), 4.16 (dd, J = 16.8, 6.1 Hz, 2H), 4.05 (dd, J = 16.8, 5.2 Hz, 2H), 2.16 (m, 1H), 2.06 (m, 1H), 1.97-1.68 (m, 7H), 1.57 (m, 3H) |
| 50 | ((2- (((3S,6S,9aS)- 3-(3- (cyanomethyl) azetidine-1- carbonyl)-5- oxooctahydro- 1H- pyrrolo[1,2- a]azepin-6- yl)carbamoyl) benzo[b]thiophen- 5-yl) difluoromethyl) phosphonic acid | 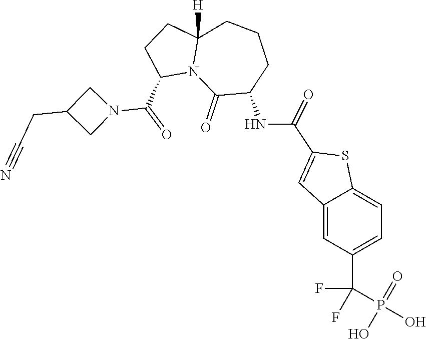 [SMILES: N#CCC1CN(C(=O)[C@@H]2CC[C@@H]3CCC[C@H](NC(=O)c4cc5cc(C(F)(F)P(=O)(O)O)ccc5s4)C(=O)N32)C1] | 581.0 [M + H]⁺ | ¹H NMR (600 MHz, DMSO-d₆) δ 8.75 (t, J = 6.2 Hz, 1H), 8.27 (d, J = 1.9 Hz, 1H), 8.10 (d, J = 8.6 Hz, 1H), 8.05 (s, 1H), 7.59-7.53 (m, 1H), 4.62 (t, J = 9.4 Hz, 1H), 4.46 (t, J = 8.4 Hz, 1H), 4.38 (dd, J = 8.1, 4.2 Hz, 1H), 4.29 (t, J = 8.5 Hz, 1H), 4.05- 3.92 (m, 3H), 3.88 (dd, J = 8.9, 4.8 Hz, 1H), 3.56 (ddd, J = 21.0, 10.0, 5.5 Hz, 2H), 2.99-2.83 (m, 3H), 2.79 (dd, J = 7.1, 2.8 Hz, 1H), 2.23-2.13 (m, 1H), 2.01-1.89 (m, 2H), 1.84 (m, 1H), 1.80-1.55 (m, 6H) |
| 51 | ((2- (((3S,6S,9aS)-3- (3-(3- cyanophenyl) azetidine-1- carbonyl)-5- oxooctahydro-1H- pyrrolo[1,2-a] azepin-6- yl)carbamoyl) benzo[b]thiophen- 5-yl) difluoromethyl) phosphonic acid | 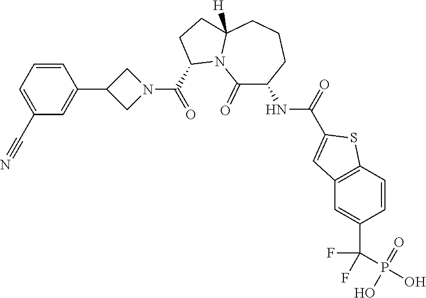 [SMILES: N#Cc1cccc(C2CN(C(=O)[C@@H]3CC[C@@H]4CCC[C@H](NC(=O)c5cc6cc(C(F)(F)P(=O)(O)O)ccc6s5)C(=O)N43)C2)c1] | 643.2 [M + H]⁺ | ¹H NMR (600 MHz, DMSO-d₆) δ 8.77 (t, J = 7.1 Hz, 1H), 8.29 (dd, J = 13.1, 2.3 Hz, 1H), 8.12 (t, J = 6.5 Hz, 1H), 8.07 (s, 1H), 7.92-7.82 (m, 1H), 7.77-7.67 (m, 2H), 7.61- 7.50 (m, 2H), 4.76 (t, J = 8.9 Hz, 1H), 4.71-4.62 (m, 1H), 4.59 (t, J = 8.8 Hz, 1H), 4.39 (dt, J = 21.7, 6.9 Hz, 2H), 4.31- 4.16 (m, 2H), 3.95 (d, J = 7.8 Hz, 2H), 3.87 (t, J = 8.6 Hz, 1H), 2.18 (d, J = 6.5 Hz, 1H), 2.04-1.92 (m, 2H), 1.89-1.63 (m, 7H) |
| 52 | ((2- (((3S,6S,10aS)- 3-((3- cyano-2H- indazol-5- yl)carbamoyl)-5- oxodecahydropyrrolo [1,2-a]azocin-6- yl)carbamoyl) benzo[b]thiophen- 5-yl) difluoromethyl) phosphonic acid | 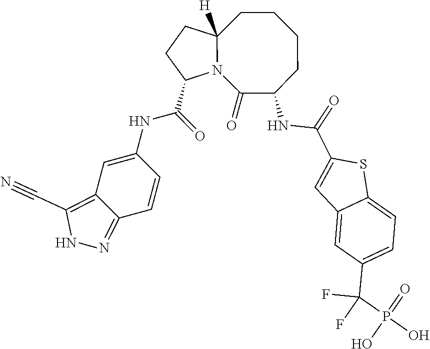 [SMILES: N#Cc1[nH]nc2ccc(NC(=O)[C@@H]3CC[C@@H]4CCCC[C@H](NC(=O)c5cc6cc(C(F)(F)P(=O)(O)O)ccc6s5)C(=O)N43)cc12] | 657.4 [M + H]⁺ | ¹H NMR (600 MHz, DMSO-d₆) δ 14.29 (s, 1H), 10.41 (s, 1H), 8.83 (d, J = 6.7 Hz, 1H), 8.35 (s, 1H), 8.27 (s, 1H), 8.09 (d, J = 8.5 Hz, 1H), 8.02 (s, 1H), 7.70 (d, J = 9.1 Hz, 1H), 7.56 (d, J = 8.7 Hz, 1H), 7.53-7.46 (m, 1H), 4.93 (s, 1H), 4.46 (t, J = 8.8 Hz, 1H), 4.33 (d, J = 10.5 Hz, 1H), 2.22 (d, J = 11.8 Hz, 1H), 2.11 (s, 1H), 2.04-1.27 (m, 11H) |
| 53 | (difluoro(2- (((3S,6S,10aS)-3- (3-fluoro-3- phenylazetidine-1- carbonyl)-5- oxodecahydropyrrolo [1,2-a]azocin-6- yl)carbamoyl) benzo[b]thiophen- 5-yl)methyl) phosphonic acid | 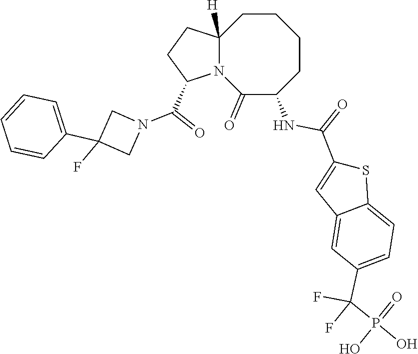 [SMILES: O=C(N[C@H]1CCCC[C@H]2CC[C@@H](C(=O)N3CC(F)(c4ccccc4)C3)N2C1=O)c1cc2cc(C(F)(F)P(=O)(O)O)ccc2s1] | 650.0 [M + H]⁺ | ¹H NMR (600 MHz, DMSO-d₆) δ 8.90 (dd, J = 20.5, 7.1 Hz, 1H), 8.30 (d, J = 26.9 Hz, 1H), 8.09 (t, J = 8.4 Hz, 1H), 8.04 (d, J = 10.5 Hz, 1H), 7.60- 7.54 (m, 1H), 7.45 (m, 4H), 5.00-4.86 (m, 2H), 4.66 (dt, J = 21.4, 11.1 Hz, 1H), 4.41-4.31 (m, 2H), 4.27 (m, 2H), 2.07-1.99 (m, 1H), 1.95-1.71 (m, 6H), 1.69-1.42 (m, 4H) |
| 54 | (difluoro(2- (((3S,6S,10aS)-3- (3-fluoro-3-(2- fluorophenyl) azetidine-1- carbonyl)-5- oxodecahydropyrrolo [1,2-a]azocin-6- yl)carbamoyl) benzo[b]thiophen- 5-yl)methyl) phosphonic acid | 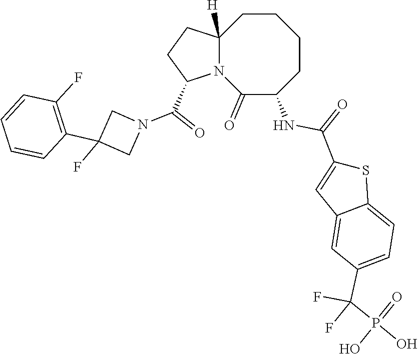 [SMILES: O=C(N[C@H]1CCCC[C@H]2CC[C@@H](C(=O)N3CC(F)(c4ccccc4F)C3)N2C1=O)c1cc2cc(C(F)(F)P(=O)(O)O)ccc2s1] | 666.2 [M − H]⁻ | ¹H NMR (600 MHz, DMSO-d₆) δ 8.88 (dd, J = 38.1, 7.2 Hz, 1H), 8.28 (d, J = 51.2 Hz, 1H), 8.10 (t J = 10.1 Hz, 1H), 8.03 (d, J = 22.5 Hz, 1H), 7.56 (tt, J = 28.9, 7.3 Hz, 3H), 7.30 (tt, J = 15.7, 9.1 Hz, 2H), 5.09-4.69 (m, 4H), 4.50 (ddd, J = 35.5, 22.6, 11.8 Hz, 2H), 4.39-4.21 (m, 3H), 2.25-2.11 (m, 1H), 2.08-1.99 (m, 1H), 1.82 (dd, J = 45.1, 31.7 Hz, 6H), 1.69-1.55 (m, 2H), 1.48 (s, 1H) |
| 55 | ((2- (((3S,6S,10aS)- 3-((4- chloroquinolin-6- yl)carbamoyl)-5- oxodecahydropyrrolo [1,2-a]azocin-6- yl)carbamoyl) benzo[b]thiophen- 5-yl) difluoromethyl) phosphonic acid | 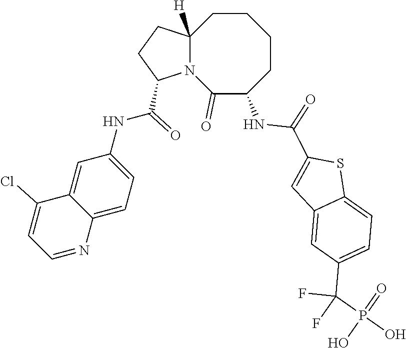 [SMILES: O=C(N[C@H]1CCCC[C@H]2CC[C@@H](C(=O)Nc3ccc4nccc(Cl)c4c3)N2C1=O)c1cc2cc(C(F)(F)P(=O)(O)O)ccc2s1] | 677.2 [M + H]⁺ | ¹H NMR (600 MHz, DMSO-d₆) δ 10.68 (s, 1H), 8.85 (d, J = 6.7 Hz, 1H), 8.77-8.62 (m, 2H), 8.29 (s, 1H), 8.13 (d, J = 8.4 Hz, 1H), 8.04 (d, J = 3.8 Hz, 2H), 7.93 (d, J = 9.5 Hz, 1H), 7.72 (d, J = 4.7 Hz, 1H), 7.57 (d, J = 8.8 Hz, 1H), 5.00-4.92 (m, 1H), 4.52 (t, J = 8.8 Hz, 1H), 4.34 (s, 1H), 2.27 (s, 1H), 2.14 (d, J = 10.5 Hz, 1H), 1.87 (s, 7H), 1.76-1.47 (m, 4H) |
| 56 | (difluoro(2- (((3S,6S,10aS)-3- (3-(4-(2- hydroxypropan-2- yl)-1H- 1,2,3-triazol-1- yl)azetidine- 1-carbonyl)-5- oxodecahydropyrrolo [1,2-a]azocin-6- yl)carbamoyl) benzo[b]thiophen- 5-yl)methyl) phosphonic acid | 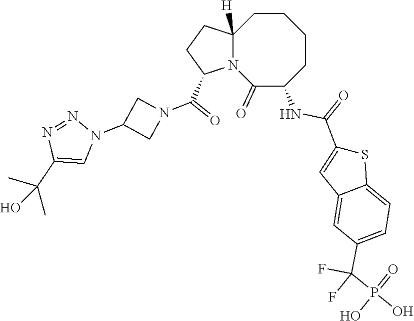 [SMILES: CC(C)(O)c1cn(C2CN(C(=O)[C@@H]3CC[C@@H]4CCCC[C@H](NC(=O)c5cc6cc(C(F)(F)P(=O)(O)O)ccc6s5)C(=O)N43)C2)nn1] | 679.2 [M − H]⁻ | ¹H NMR (600 MHz, DMSO-d₆) δ 8.86 (d, J = 7.7 Hz, 1H), 8.45 (d, J = 8.0 Hz, 1H), 8.29 (d, J = 12.6 Hz, 1H), 8.13-7.98 (m, 2H), 7.57 (d, J = 8.8 Hz, 1H), 5.64 (d, J = 15.4 Hz, 1H), 5.50 (d, J = 8.0 Hz, 1H), 5.04 (d, J = 19.7 Hz, 1H), 5.02-4.91 (m, 1H), 4.73 (d, J = 6.8 Hz, 1H), 4.54-4.43 (m, 1H), 4.34 (p, J = 9.8, 9.1 Hz, 1H), 4.26 (dd, J = 11.1, 6.4 Hz, 1H), 4.16 (dd, J = 10.9, 4.9 Hz, 1H), 2.52 (s, 7H), 2.17 (d, J = 11.1 Hz, 1H), 2.03 (d, J = 20.2 Hz, 2H), 1.86 (dd, J = 26.3, 13.9 Hz, 3H), 1.75 (d, J = |
| | | | | 11.4 Hz, 1H), 1.70-1.55 |
| | | | | (m, 2H), 1.51 (s, 1H), |
| | | | | 1.47-1.35 (m, 1H), 1.15 |
| | | | | (q, J = 7.4 Hz, 1H) |
| 57 | ((2- (((3S,6S,10aS)- 3-((1,1- dioxido-2,3- dihydrobenzo[b] thiophen-5-yl) carbamoyl)-5- oxodecahydropyrrolo [1,2-a]azocin-6- yl)carbamoyl) benzo[b]thiophen-5- yl)difluoromethyl) phosphonic acid | 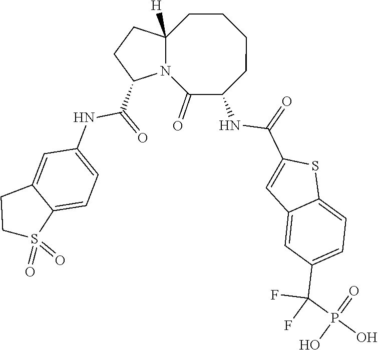 [SMILES: O=C(N[C@H]1CCCC[C@H]2CC[C@@H](C(=O)Nc3ccc4c(c3)CCS4(=O)=O)N2C1=O)c1cc2cc(C(F)(F)P(=O)(O)O)ccc2s1] | 682.2 [M + H]⁺ | ¹H NMR (600 MHz, DMSO-d₆) δ 10.57 (s, 1H), 8.86 (d, J = 6.7 Hz, 1H), 8.29 (s, 1H), 8.12 (d, J = 8.6 Hz, 1H), 8.05 (s, 1H), 7.89 (s, 1H), 7.66 (d, J = 8.5 Hz, 1H), 7.57 (d, J = 8.4 Hz, 2H), 4.99-4.90 (m, 1H), 4.46 (t, J = 8.6 Hz, 1H), 4.33 (t, J = 9.6 Hz, 1H), 3.54 (t, J = 6.9 Hz, 2H), 3.31 (t, J = 6.9 Hz, 2H), 2.24 (dt, J = 11.8, 7.3 Hz, 1H), 2.18- 2.06 (m, 1H), 2.00-1.49 (m, 10H) |
| 58 | ((2- (((3S,6S,10aS)-3- ((1,1-dioxidothio- chroman-6-yl) carbamoyl)-5- oxodecahydropyrrolo [1,2-a]azocin-6- yl)carbamoyl) benzo[b]thiophen- 5-yl)difluoro- methyl)phosphonic acid | 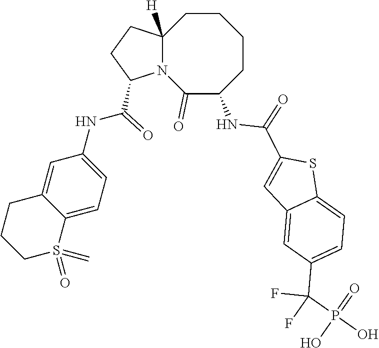 [SMILES: C=S1(=O)CCCc2cc(NC(=O)[C@@H]3CC[C@@H]4CCCC[C@H](NC(=O)c5cc6cc(C(F)(F)P(=O)(O)O)ccc6s5)C(=O)N43)ccc21] | 696.2 [M + H]⁺ | ¹H NMR (600 MHz, DMSO-d₆) δ 10.46 (s, 1H), 8.86 (d, J = 6.7 Hz, 1H), 8.29 (s, 1H), 8.12 (d, J = 8.5 Hz, 1H), 8.05 (s, 1H), 7.71 (d, J = 8.6 Hz, 1H), 7.65 (d, J = 2.0 Hz, 1H), 7.57 (d, J = 8.4 Hz, 2H), 4.93 (q, J = 8.4, 7.7 Hz, 1H), 4.44 (t, J = 8.6 Hz, 1H), 4.33 (d, J = 9.9 Hz, 1H), 3.48-3.40 (m, 2H), 2.95 (m, 2H), 2.45- 2.18 (m, 4H), 2.09 (m, 1H), 2.03-1.43 (m, 11H) |
| 59 | (difluoro(2- (((3S,6S,10aS)-3- ((3-methyl-2H- indazol-5- yl)carbamoyl)-5- oxodecahydro- pyrrolo[1,2-a] azocin-6- yl)carbamoyl) benzo[b]thiophen- 5-yl)methyl) phosphonic acid | 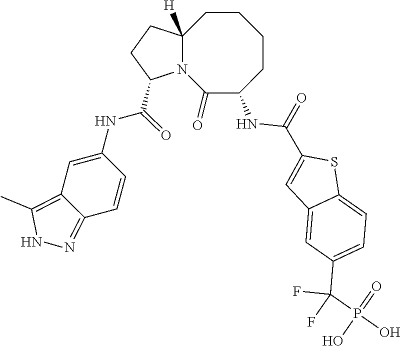 [SMILES: Cc1[nH]nc2ccc(NC(=O)[C@@H]3CC[C@@H]4CCCC[C@H](NC(=O)c5cc6cc(C(F)(F)P(=O)(O)O)ccc6s5)C(=O)N43)cc12] | 646.0 [M + H]⁺ | ¹H NMR (600 MHz, DMSO-d₆) δ 12.52 (s, 1H), 10.08 (s, 1H), 8.84 (d, J = 6.8 Hz, 1H), 8.30 (s, 1H), 8.12 (t, J = 8.2 Hz, 1H), 8.10-7.83 (m, 2H), 7.57 (d, J = 8.6 Hz, 1H), 7.48-7.24 (m, 2H), 5.02- 4.90 (m, 1H), 4.47 (m, 1H), 4.32 (t, J = 9.7 Hz, 1H), 2.44 (s, 3H), 2.23 (m, 1H), 2.10 (m, 1H), 2.08-1.27 (m, 10H) |
| 60 | ((2- (((4S,7S,10aS)-4- (3-(1H- imidazol-1- yl)azetidine-1- carbonyl)-6- oxodecahydropyrido [1,2-a]azepin-7- yl)carbamoyl) benzo[b]thiophen- 5-yl) difluoromethyl) phosphonic acid | 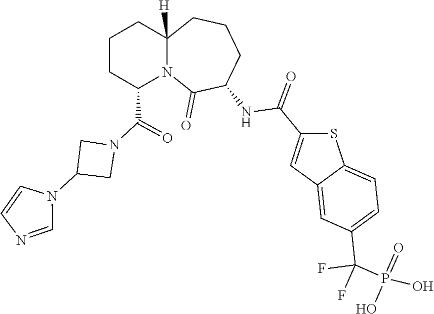 [SMILES: O=C(N[C@H]1CCC[C@H]2CCC[C@@H](C(=O)N3CC(n4ccnc4)C3)N2C1=O)c1cc2cc(C(F)(F)P(=O)(O)O)ccc2s1] | 622.2 [M + H]⁺ | ¹H NMR (600 MHz, DMSO-d₆) δ 8.75 (s, 1H), 8.53 (app. d, J = 35.4 Hz, 1H), 8.26 (m, 1H), 8.04 (m, 2H), 7.81-7.60 (m, 1H), 7.55 (s, 1H), 7.25 (d, J = 15.5 Hz, 1H), 5.24 (m, 1H), 5.02 (s, 1H), 4.85- 4.50 (m, 3H), 4.31 (m, 1H), 4.06 (m, 4H), 2.28 (s, 1H), 1.91 (s, 2H), 1.75 (m, 4H), 1.60 (m, 2H), 1.42 (m, 2H) |
| 61 | (difluoro(2- (((3S,6S,10aS)- 3-(3-(1-imino-1- oxidohexahydro-1l6- thiopyran-4- yl)azetidine- 1-carbonyl)-5- oxodecahydropyrrolo [1,2-a]azocin-6- yl)carbamoyl) benzo[b] thiophen-5- yl)methyl) phosphonic acid | 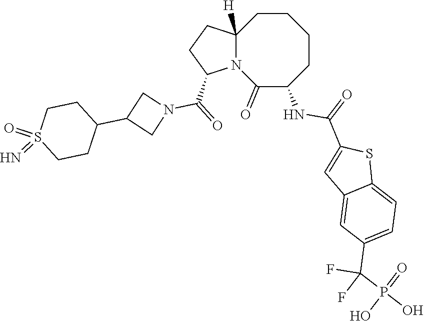 [SMILES: N=S1(=O)CCC(C2CN(C(=O)[C@@H]3CC[C@@H]4CCCC[C@H](NC(=O)c5cc6cc(C(F)(F)P(=O)(O)O)ccc6s5)C(=O)N43)C2)CC1] | 687.2 [M + H]⁺ | ¹H NMR (600 MHz, DMSO-d₆) δ 8.81 (app. m, 1H), 8.34-8.27 (m, 1H), 8.09 (d, J = 8.6 Hz, 1H), 8.05 (d, J = 7.0 Hz, 1H), 7.57 (d, J = 8.6 Hz, 1H), 4.90 (m, 1H), 4.49- 4.38 (m, 1H), 4.29-4.21 (m, 2H), 4.16 (m, 1H), 4.05 (dt, J = 16.7, 7.4 Hz, 1H), 3.89 (m, 1H), 3.85 (m, 1H), 3.79 (m, 2H), 3.58 (m, 2H), 3.51 (m, 1H), 3.22 (m, 2H), 3.10- 3.01 (m, 2H), 2.10 (m, 1H), 2.05-1.97 (m, 1H), 1.84 (m, 7H), 1.71 (m, 1H), 1.62 (m, 1H), 1.58- 1.52 (m, 1H), 1.48 (m, 3H) |
| 62 | ((2- (((4S,7S,11aS)- 4-(3-(1H- imidazol-1- yl)azetidine- 1-carbonyl)-6- oxodecahydro-2H- pyrido[1,2-a] azocin-7- yl)carbamoyl) benzo[b]thiophen- 5-yl) difluoromethyl) phosphonic acid | 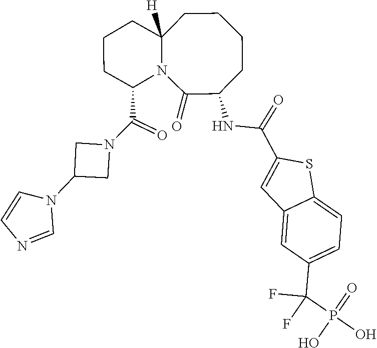 [SMILES: O=C(N[C@H]1CCCC[C@H]2CCC[C@@H](C(=O)N3CC(n4ccnc4)C3)N2C1=O)c1cc2cc(C(F)(F)P(=O)(O)O)ccc2s1] | 636.2 [M + H]⁺ | ¹H NMR (600 MHz, DMSO-d₆) δ 9.04 (app. m, 1H), 8.61 (s, 1H), 8.39- 7.61 (m, 4H), 7.59- 7.28 (m, 2H), 5.17 (m, 2H), 4.60 (s, 1H), 4.42 (s, 1H), 4.22 (s, 1H), 4.11- 3.85 (m, 2H), 3.78 (m, 1H), 2.13 (s, 2H), 1.87 (s, 3H), 1.64 (m, 6H), 1.44- 1.11 (m, 2H), 1.04 (s, 1H) |
| 63 | ((2- (((4S,7S,11aS)- 4-(rel-(trans)- 3-cyano-4- phenylpyrrolidine- 1-carbonyl)-6- oxodecahydro-2H- pyrido[1,2-a] azocin-7- yl)carbamoyl) benzo[b]thiophen- 5-yl) difluoromethyl) phosphonic acid | 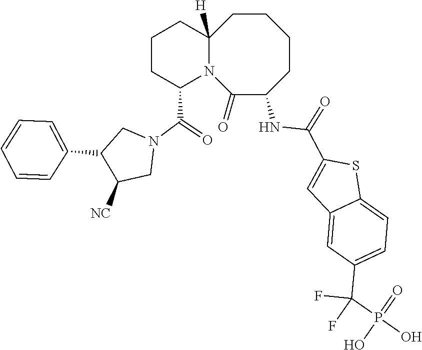 | 683.2 [M − H]⁻ | ¹H NMR (600 MHz, DMSO-d₆) δ 8.87-8.69 (m, 1H), 8.38-8.27 (m, 1H), 8.15-8.01 (m, 2H), 7.63-7.52 (m, 1H), 7.38 (m, 5H), 5.04 (m, 1H), 4.88 (m, 1H), 4.41-4.24 (m, 2H), 4.16-3.88 (m, 5H), 2.11-1.56 (m, 10H), 1.41 (m, 4H) |
| 64 | ((2- (((3S,6S,10aS)- 3-(3-(4- ethynyloxazol-5- yl)azetidine-1- carbonyl)-5- oxodecahydropyrrolo [1,2-a]azocin-6- yl)carbamoyl) benzo[b]thiophen- 5-yl) difluoromethyl) phosphonic acid | 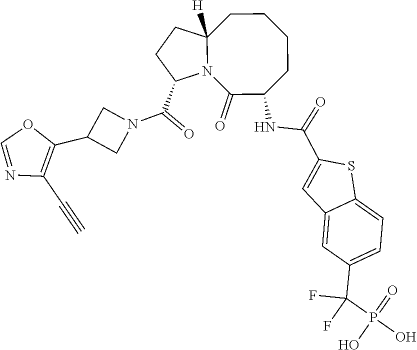 [SMILES: C#Cc1ncoc1C1CN(C(=O)[C@@H]2CC[C@@H]3CCCC[C@H](NC(=O)c4cc5cc(C(F)(F)P(=O)(O)O)ccc5s4)C(=O)N32)C1] | 647.0 [M + H]⁺ | ¹H NMR (600 MHz, DMSO-d₆) δ 8.86 (app. m, 1H), 8.44-8.01 (m, 4H), 7.56 (d, J = 8.5 Hz, 1H), 4.93 (s, 1H), 4.78 (s, 1H), 4.49 (d, J = 15.6 Hz, 2H), 4.28 (s, 3H), 4.14 (s, 1H), 4.04 (s, 1H), 3.97 (s, 1H), 2.14 (s, 1H), 2.02 (s, 1H), 1.83 (s, 6H), 1.72 (s, 1H), 1.63 (s, 1H), 1.57 (s, 1H), 1.49 (s, 1H) |
| 65 | (difluoro(2- (((3S,6S,10S)-3- (3-(1- methyl-1H- pyrazol-3- yl)azetidine- 1-carbonyl)-5- oxodecahydropyrrolo [1,2-a]azocin-6- yl)carbamoyl) benzo[b]thiophen-5- yl)methyl) phosphonic acid | 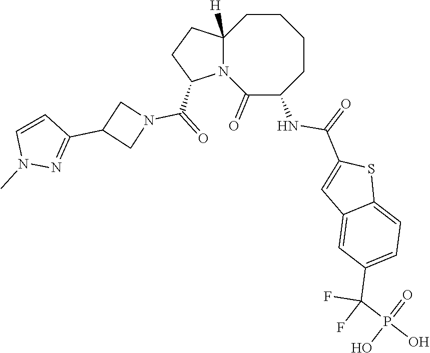 [SMILES: Cn1ccc(C2CN(C(=O)[C@@H]3CC[C@@H]4CCCC[C@H](NC(=O)c5cc6cc(C(F)(F)P(=O)(O)O)ccc6s5)C(=O)N43)C2)n1] | 636.2 [M + H]⁺ | ¹H NMR (600 MHz, DMSO-d₆) δ 8.85 (d, J = 6.9 Hz, 1H), 8.31 (d, J = 8.0 Hz, 1H), 8.11 (dd, J = 8.6, 3.6 Hz, 1H), 8.05 (d, J = 6.1 Hz, 1H), 7.65- 7.50 (m, 2H), 6.17 (dd, J = 12.1, 2.2 Hz, 1H), 4.92 (m, 1H), 4.60 (m, 1H), 4.41-4.19 (m, 3H), 4.18- 4.08 (m, 1H), 3.96- 3.78 (m, 2H), 3.75 (app. d, 3H), 2.14 (m, 1H), 2.02 (m, 1H), 1.99-1.73 (m, 6H), 1.71 (m, 1H), 1.63 (m, 1H), 1.56 (m, 1H), 1.49 (m, 1H) |
| 66 | (difluoro(2- (((3S,6S,10aS)- 3-(3-((S- methyl- sulfonimidoyl) methyl) azetidine-1- carbonyl)-5- oxodecahydropyrrolo [1,2-a]azocin-6- yl)carbamoyl) benzo[b]thiophen-5- yl)methyl) phosphonic acid | 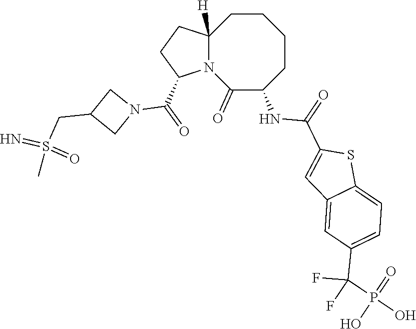 [SMILES: CS(=N)(=O)CC1CN(C(=O)[C@@H]2CC[C@@H]3CCCC[C@H](NC(=O)c4cc5cc(C(F)(F)P(=O)(O)O)ccc5s4)C(=O)N32)C1] | 647.2 [M + H]⁺ | ¹H NMR (600 MHz, DMSO-d₆) δ 8.83 (dd, J = 17.3, 7.0 Hz, 1H), 8.30 (s, 1H), 8.09 (d, J = 8.5 Hz, 1H), 8.04 (s, 1H), 7.56 (d, J = 8.6 Hz, 1H), 4.92 (m, 1H), 4.55-4.31 (m, 1H), 4.24 (m, 2H), 4.10-3.92 (m, 2H), 3.72 (m, 2H), 3.53 (app. d, J = 7.5 Hz, 1H), 3.42 (m, 1H), 3.09 (m, 1H), 2.96 (app. d, J = 5.8 Hz, 3H), 2.30-1.89 (m, 3H), 1.82 (m, 5H), 1.71 (m, 1H), 1.63 (dm, 1H), 1.55 (m, 1H), 1.49 (m, 1H) |
| 67 | ((2- (((3S,6S,10aS)-3- (3-(1H-imidazol-4- yl)azetidine-1- carbonyl)-5- oxodecahydro- pyrrolo[1,2-a] azocin-6- yl)carbamoyl) benzo[b]thiophen- 5-yl) difluoromethyl) phosphonic acid | 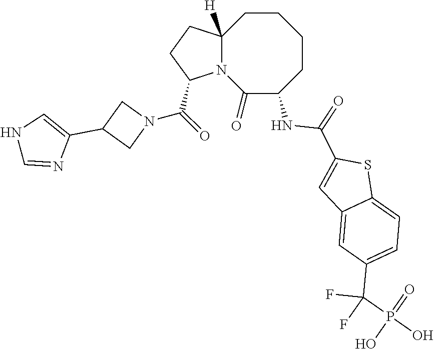 [SMILES: O=C(N[C@H]1CCCC[C@H]2CC[C@@H](C(=O)N3CC(c4c[nH]cn4)C3)N2C1=O)c1cc2cc(C(F)(F)P(=O)(O)O)ccc2s1] | 622.2 [M + H]⁺ | |
| 68 | (difluoro(2- (((3S,6S,10aS)-3- (3-(4-methyl-1H- pyrazol-1- yl)azetidine-1- carbonyl)-5- oxodecahydropyrrolo [1,2-a]azocin-6- yl)carbamoyl) benzo[b]thiophen- 5-yl)methyl) phosphonic acid | 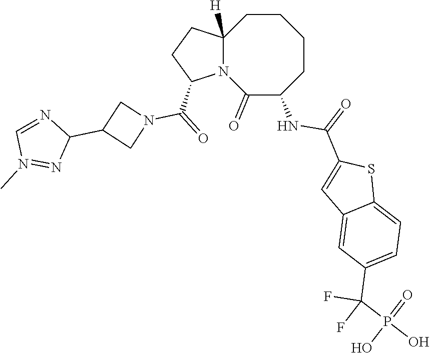 | 636.0 [M + H]⁺ | ¹H NMR (600 MHz, DMSO-d₆) δ 8.87 (dd, J = 17.7, 7.0 Hz, 1H), 8.31 (s, 1H), 8.12-8.03 (m, 2H), 7.57 (m, 2H), 7.33 (d, J = 16.8 Hz, 1H), 5.17 (m, 1H), 4.93 (s, 1H), 4.87- 4.50 (m, 2H), 4.43-3.96 (m, 6H), 2.15 (s, 1H), 2.07-2.00 (m, 1H), 1.98 (app. m, 4H), 1.83 (m, 6H), 1.73 (m, 1H), 1.56 (m, 3H) |
| 69 | (difluoro(2- (((3S,6S,10aS)- 3-(3-((1- methyl-1H- pyrazol-5- yl)methylene) azetidine-1- carbonyl)-5- oxodecahydropyrrolo [1,2-a]azocin-6- yl)carbamoyl) benzo[b] thiophen-5- yl)methyl) phosphonic acid | 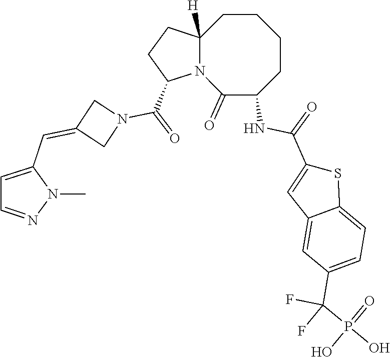 [SMILES: Cn1nccc1C=C1CN(C(=O)[C@@H]2CC[C@@H]3CCCC[C@H](NC(=O)c4cc5cc(C(F)(F)P(=O)(O)O)ccc5s4)C(=O)N32)C1] | 648.0 [M + H]⁺ | ¹H NMR (600 MHz, DMSO-d₆) δ 8.84 (d, J = 7.1 Hz, 1H), 8.29 (d, J = 6.5 Hz, 1H), 8.10 (app. dd, J = 8.5, 3.7 Hz, 1H), 8.03 (d, J = 4.9 Hz, 1H), 7.60-7.51 (m, 1H), 7.34 (d, J = 15.8 Hz, 1H), 6.55 (s, 1H), 6.08 (d, J = 36.3 Hz, 1H), 5.19 (s, 1H), 4.91 (m, 2H), 4.76-4.48 (m, 2H), 4.36 (m, 1H), 4.25 (s, 1H), 3.77 (d, J = 4.1 Hz, 3H), 2.17 (m, 1H), 2.09-1.99 (m, 1H), 1.93- 1.70 (m, 7H), 1.57 (m, 3H) |
| 70 | (difluoro(2- (((3S,6S,10aS)- 3-(2-imino-2- oxido-2l6- thia-6- azaspiro[3.3] heptane-6- carbonyl)-5- oxodecahydropyrrolo [1,2-a]azocin-6- yl)carbamoyl) benzo[b]thiophen- 5-yl)methyl) phosphonic acid | 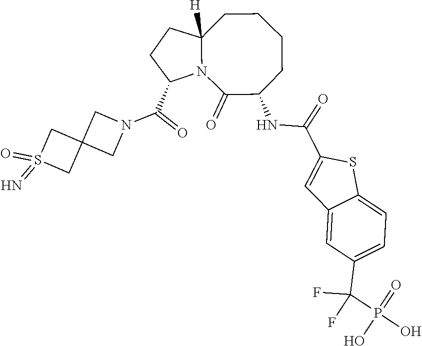 [SMILES: N=S1(=O)CC2(CN(C(=O)[C@@H]3CC[C@@H]4CCCC[C@H](NC(=O)c5cc6cc(C(F)(F)P(=O)(O)O)ccc6s5)C(=O)N43)C2)C1] | 645.2 [M + H]⁺ | ¹H NMR (600 MHz, DMSO-d₆) δ 8.91-8.80 (m, 1H), 8.30 (s, 1H), 8.11 (d, J = 8.6 Hz, 1H), 8.05 (s, 1H), 7.57 (d, J = 8.6 Hz, 1H), 4.92 (br. s, 1H), 4.56 (t, J = 8.5 Hz, 1H), 4.35 (dd, J = 12.2, 8.9 Hz, 1H), 4.29-4.19 (m, 4H), 4.15-3.96 (m, 4H), 2.16-2.09 (m, 1H), 2.02 (m, 1H), 1.81 (m, 6H), 1.71 (m, 1H), 1.62 (s, 1H), 1.55 (m, 1H), 1.48 (s, 1H) |
| 71 | ((2- (((3S,6S,10aS)- 3-(6,6- dioxido-6- thia-2,7- diazaspiro[3.4] octane-2- carbonyl)-5- oxodecahydropyrrolo [1,2-a]azocin-6- yl)carbamoyl) benzo[b]thiophen-5- yl)difluoromethyl) phosphonic acid | 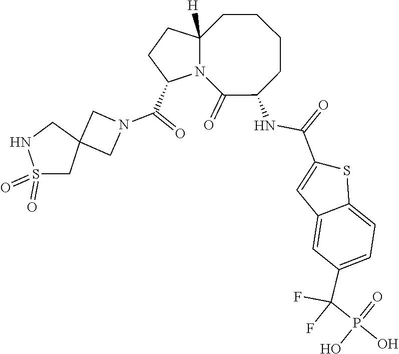 [SMILES: O=C(N[C@H]1CCCC[C@H]2CC[C@@H](C(=O)N3CC4(CNS(=O)(=O)C4)C3)N2C1=O)c1cc2cc(C(F)(F)P(=O)(O)O)ccc2s1] | 661.0 [M + H]⁺ | ¹H NMR (600 MHz, DMSO-d₆) δ 8.87 (app. t, J = 6.3 Hz, 1H), 8.30 (s, 1H), 8.10 (d, J = 8.6 Hz, 1H), 8.05 (s, 1H), 7.57 (d, J = 8.5 Hz, 1H), 7.12 (dt, J = 13.3, 6.3 Hz, 1H), 4.92 (dt, J = 11.8, 6.1 Hz, 1H), 4.40 (m, 2H), 4.24 (m, 3H), 4.15 (d, J = 8.9 Hz, 1H), 3.96 (d, J = 10.1 Hz, 1H), 3.89 (m, 2H), 3.41 (d, J = 5.7 Hz, 1H), 3.37 (d, J = 6.1 Hz, 1H), 3.33 (t, J = 6.0 Hz, 1H), 2.17- 2.09 (m, 1H), 2.08-1.99 (m, 1H), 1.81 (m, 6H), 1.71 (m, 1H), 1.62 (m, 1H), 1.56 (m, 1H), 1.48 (s, 1H) |
| 72 | ((2- (((3S,6S,10aS)-3- (7-cyclopropyl- 5-oxa-2,6- diazaspiro[3.4] oct-6-ene- 2-carbonyl)-5- oxodecahydropyrrolo [1,2-a]azocin-6- yl)carbamoyl) benzo[b]thiophen- 5-yl) difluoromethyl) phosphonic acid | 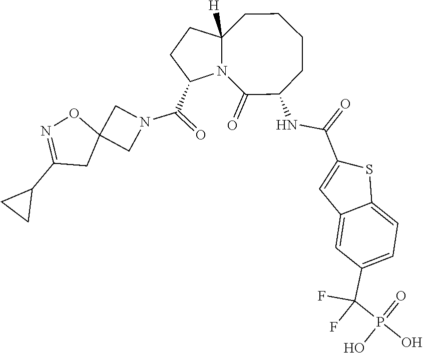 [SMILES: O=C(N[C@H]1CCCC[C@H]2CC[C@@H](C(=O)N3CC4(CC(C5CC5)=NO4)C3)N2C1=O)c1cc2cc(C(F)(F)P(=O)(O)O)ccc2s1] | 651.0 [M + H]⁺ | ¹H NMR (600 MHz, DMSO-d₆) δ 8.85 (m, 1H), 8.30 (d, J = 7.1 Hz, 1H), 8.08 (m, 2H), 7.58 (d, J = 8.6 Hz, 1H), 4.92 (s, 1H), 4.55 (app. t, J = 10.6 Hz, 1H), 4.25 (m, 3H), 4.09 (d, J = 10.9 Hz, 1H), 3.96 (s, 1H), 3.87 (d, J = 10.9 Hz, 1H), 3.12 (m, 2H), 2.08 (m, 2H), 1.97- 1.27 (m, 10H), 0.81 (m, 2H), 0.72 (m, 2H) |
| 73 | (difluoro(2- (((3S,6S,10aS)- 3-(3-(S- methylsulfonimidoyl) azetidine-1- carbonyl)-5- oxodecahydropyrrolo [1,2-a]azocin-6- yl)carbamoyl) benzo[b]thiophen- 5-yl)methyl) phosphonic acid | 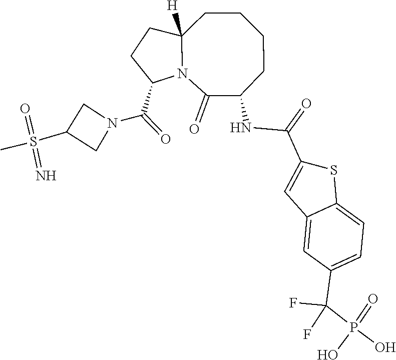 [SMILES: CS(=N)(=O)C1CN(C(=O)[C@@H]2CC[C@@H]3CCCC[C@H](NC(=O)c4cc5cc(C(F)(F)P(=O)(O)O)ccc5s4)C(=O)N32)C1] | 633.2 [M + H]⁺ | ¹H NMR (600 MHz, DMSO-d₆) δ 8.87 (m, 1H), 8.31 (d, J = 4.8 Hz, 1H), 8.11 (d, J = 8.5 Hz, 1H), 8.05 (s, 1H), 7.56 (d, J = 8.6 Hz, 1H), 4.91 (t, J = 9.0 Hz, 1H), 4.70-4.51 (m, 2H), 4.46-4.36 (m, 1H), 4.31 (dd, J = 9.4, 5.2 Hz, 1H), 4.25 (q, J = 7.8, 6.9 Hz, 2H), 4.20-4.15 (m, 1H), 4.12-4.07 (m, 1H), 4.03 (m, 1H), 3.98- 3.94 (m, 1H), 2.90 (m, 3H), 2.20-2.10 (m, 1H), 2.03 (m, 1H), 1.93-1.69 (m, 6H), 1.69-1.17 (m, 4H) |
| 74 | ((2- (((3S,6S,11aS)-3- ((rel- trans)-3- cyano-4- phenylpyrrolidine- 1-carbonyl)-5- oxodecahydro-1H- pyrrolo[1,2- a]azonin-6- yl)carbamoyl) benzo[b]thiophen- 5-yl) difluoromethyl) phosphonic acid | 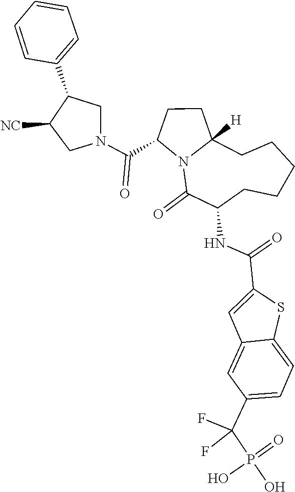 [SMILES: N#C[C@@H]1CN(C(=O)[C@@H]2CC[C@@H]3CCCCC[C@H](NC(=O)c4cc5cc(C(F)(F)P(=O)(O)O)ccc5s4)C(=O)N32)C[C@H]1c1ccccc1] | 685.0 [M + H]⁺ | ¹H NMR (600 MHz, DMSO-d₆) δ 8.88 (m, 1H), 8.31 (d, J = 16.6 Hz, 1H), 8.12-8.00 (m, 3H), 7.56 (t, J = 8.4 Hz, 1H), 7.45-7.27 (m, 4H), 6.91 (d, J = 9.1 Hz, 1H), 5.00 (m, 1H), 4.59 (m, 1H), 4.29-4.23 (m, 1H), 4.23- 4.16 (m, 1H), 4.11 (dd, J = 10.2, 7.6 Hz, 1H), 4.02 (m, 1H), 3.94 (app. t, J = 10.0 Hz, 1H), 3.83-3.75 (m, 1H), 3.67 (t, J = 10.2 Hz, 1H), 3.61-3.53 (m, 1H), 3.48 (dd, J = 11.5, 9.5 Hz, 1H), 3.25 (app, t, J = 11.0 Hz, 1H), 3.04 (m, 1H), 2.21 (m, 1H), 2.07 (m, 1H), 1.98 (m, 1H), 1.92 (m, 2H), 1.83-1.60 (m, 4H), 1.51 (s, 1H), 1.41 (s, 1H) |
| 75 | ((2- (((3S,6S,11aS)- 3-(rel- (trans)- 3-cyano-4- phenylpyrrolidine- 1-carbonyl)-5- oxodecahydro-1H- pyrrolo[1,2-a] azonin-6- yl)carbamoyl) benzo[b]thiophen- 5-yl) difluoromethyl) phosphonic acid | 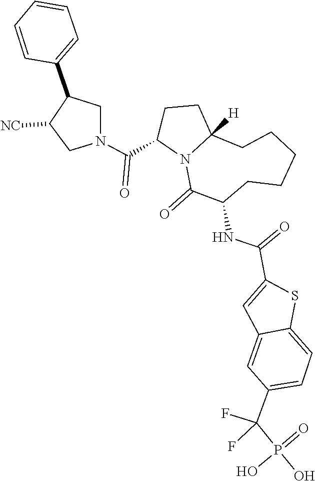 [SMILES: N#C[C@H]1CN(C(=O)[C@@H]2CC[C@@H]3CCCCC[C@H](NC(=O)c4cc5cc(C(F)(F)P(=O)(O)O)ccc5s4)C(=O)N32)C[C@@H]1c1ccccc1] | 685.0 [M + H]⁺ | ¹H NMR (600 MHz, DMSO-d₆) δ 8.89 (m, 1H), 8.32 (d, J = 2.9 Hz, 1H), 8.12 (app. t, J = 9.3 Hz, 1H), 8.05 (d, J = 5.4 Hz, 1H), 7.57 (app. t, J = 7.5 Hz, 1H), 7.42 (t, J = 8.6 Hz, 2H), 7.37 (t, J = 7.5 Hz, 2H), 7.31 (dd, J = 8.3, 5.9 Hz, 1H), 4.99 (m, 1H), 4.62 (m, 1H), 4.34 (m, 1H), 4.24 (m, 1H), 3.95 (dd, J = 11.6, 7.1 Hz, 1H), 3.89-3.81 (m, 1H), 3.74-3.62 (m, 3H), 3.56- 3.49 (m, 1H), 3.33 (dd, J = 11.8, 9.7 Hz, 1H), 2.20 (m, 1H), 2.12-2.03 (m, 1H), 2.00-1.86 (m, 4H), 1.83-1.71 (m, 3H), 1.71- 1.61 (m, 2H), 1.52- 1.48 (m, 1H), 1.44-1.38 (m, 1H) |
| 76 | ((2- (((1R,3S,6S, 11aS)-3-(rel- (trans)- 3-cyano-4- phenylpyrrolidine-1- carbonyl)-1- hydroxy-5- oxodecahydro-1H- pyrrolo[1,2- a]azonin-6- yl)carbamoyl) benzo[b]thiophen- 5-yl)difluoromethyl) phosphonic acid | 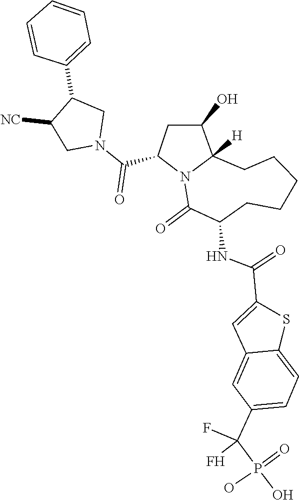 | 701.0 [M + H]⁺ | ¹H NMR (600 MHz, DMSO-d₆) δ 8.88-8.78 (m, 1H), 8.37-8.31 (m, 1H), 8.10 (app. t, J = 9.3 Hz, 1H), 8.04 (d, J = 9.0 Hz, 1H), 7.56 (app. t, J = 7.7 Hz, 1H), 7.45-7.36 (m, 4H), 7.35-7.29 (m, 1H), 5.16 (m, 1H), 5.09- 5.01 (m, 1H), 4.75-4.64 (m, 1H), 4.45-4.32 (m, 1H), 4.09-3.98 (m, 2H), 3.95-3.85 (m, 1H), 3.84- 3.74 (m, 1H), 3.75- 3.68 (m, 1H), 3.61-3.49 (m, 2H), 2.15-2.00 (m, 2H), 1.98-1.90 (m, 1H), 1.86 (m, 3H), 1.61 (m, 2H), 1.54-1.45 (m, 2H), 1.45-1.36 (m, 1H) |
| 77 | (difluoro(2- (((1R,3S,6S, 11aS)-1- hydroxy-3-(3- (morpholine-4- carbonyl)azetidine- 1-carbonyl)-5- oxodecahydro-1H- pyrrolo[1,2-a] azonin-6- yl)carbamoyl) benzo[b]thiophen- 5-yl)methyl) phosphonic acid | 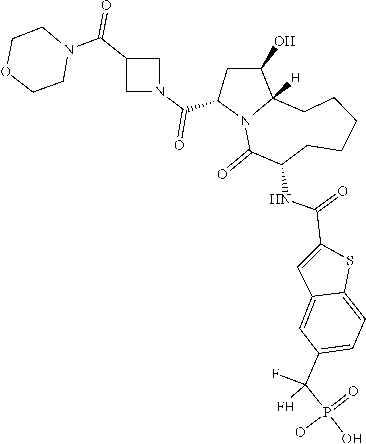 | 699.0 [M + H]⁺ | ¹H NMR (600 MHz, DMSO-d₆) δ 8.89-8.77 (m, 2H), 8.33 (d, J = 5.6 Hz, 1H), 8.09 (d, J = 8.6 Hz, 1H), 8.03 (s, 1H), 7.57 (d, J = 8.6 Hz, 1H), 7.16-6.98 (m, 1H), 5.17 (app. d, J = 13.6 Hz, 1H), 5.03 (m, 1H), 4.62 (t, J = 8.7 Hz, 1H), 4.52 (t, J = 7.5 Hz, 1H), 4.42 (m, 1H), 4.35-4.24 (m, 1H), 4.08 (t, J = 9.4 Hz, 1H), 4.00 (m, 1H), 3.95-3.83 (m, 2H), 3.74-3.64 (m, 1H), 3.52 (m, 3H), 3.45 (m, 2H), 2.04-1.91 (m, 3H), 1.87-1.75 (m, 3H), 1.66- 1.55 (m, 2H), 1.53- 1.43 (m, 3H), 1.39 (s, 1H) |
| 78 | ((2- (((3S,6S,9aS)-5- oxo-3-(3- (pyridin-3- yl)azetidine-1- carbonyl)octahydro- 1H-pyrrolo[1,2- a]azepin-6- yl)carbamoyl) benzo[b]thiophen- 5-yl)methyl) phosphonic acid | 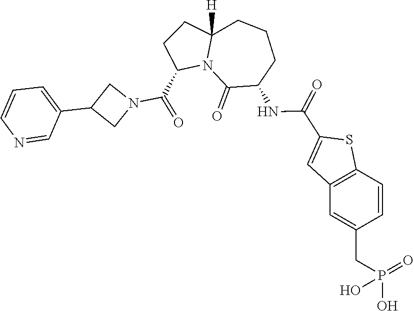 [SMILES: O=C(N[C@H]1CCC[C@H]2CC[C@@H](C(=O)N3CC(c4cccnc4)C3)N2C1=O)c1cc2cc(CP(=O)(O)O)ccc2s1] | 583.2 [M + H]⁺ | ¹H NMR (400 MHz, DMSO-d₆) δ 8.83-8.69 (m, 1H), 8.61-8.54 (m, 1H), 8.51-8.42 (m, 1H), 8.19 (s, 1H), 7.96-7.83 (m, 2H), 7.84-7.76 (m, 1H), 7.47-7.30 (m, 2H), 4.86-4.58 (m, 2H), 4.45- 4.38 (m, 1H), 4.35- 4.18 (m, 2H), 4.02-3.97 (m, 1H), 3.95-3.88 (m, 2H), 3.11 (d, J = 21.1 Hz, 2H), 2.24-2.15 (m, 1H), 2.09-1.93 (m, 2H), 1.88- 1.66 (m, 7H) |
| 79 | ((7- (((3S,6S,10aS)- 3-((3S,4R or 3R,4S)- 3-cyano-4-(2- oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphonic acid | 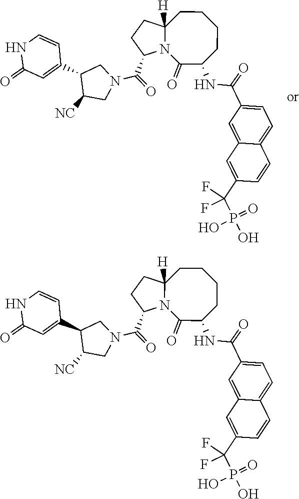 [SMILES: N#C[C@@H]1CN(C(=O)[C@@H]2CC[C@@H]3CCCC[C@H](NC(=O)c4ccc5ccc(C(F)(F)P(=O)(O)O)cc5c4)C(=O)N32)C[C@H]1c1cc[nH]c(=O)c1 ; N#C[C@H]1CN(C(=O)[C@@H]2CC[C@@H]3CCCC[C@H](NC(=O)c4ccc5ccc(C(F)(F)P(=O)(O)O)cc5c4)C(=O)N32)C[C@@H]1c1cc[nH]c(=O)c1] | 682.2 [M + H]⁺ | ¹H NMR (400 MHz, DMSO-d₆) δ 8.73-8.52 (m, 2H), 8.26-8.14 (m, 1H), 8.14-7.98 (m, 3H), 7.80-7.65 (m, 1H), 7.44- 7.29 (m, 1H), 6.42- 6.33 (m, 1H), 6.30-6.18 (m, 1H), 5.07-4.92 (m, 1H), 4.58-4.49 (m, 1H), 4.39-4.28 (m, 1H), 4.20- 4.07 (m, 1H), 4.02- 3.91 (m, 1H), 3.87-3.78 (m, 1H), 3.68-3.59 (m, 1H), 3.53-3.42 (m, 1H), 3.32-3.14 (m, 1H), 2.36- 2.18 (m, 1H), 2.16- 2.01 (m, 1H), 2.00-1.73 (m, 7H), 1.70-1.45 (m, 3H) |
| | | SFC peak 1 was used for | | |
| | | biological testing | | |
| 80 | ((7- (((3S,6S,10aS)- 3-((3R,4S or 3S,4R)-3- cyano-4-(2- oxo-1,2- dihydropyridin- 4- yl)pyrrolidine-1- carbonyl)-5- oxodecahydropyrrolo [1,2-a]azocin-6- yl)carbamoyl) naphthalen-2- yl)difluoromethyl) phosphonic acid | 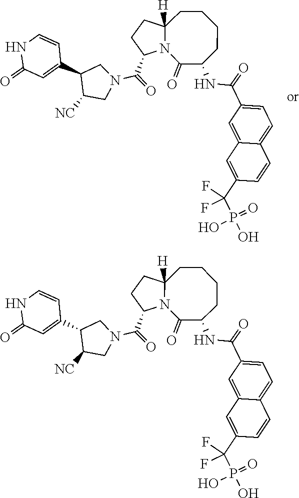 [SMILES: N#C[C@H]1CN(C(=O)[C@@H]2CC[C@@H]3CCCC[C@H](NC(=O)c4ccc5ccc(C(F)(F)P(=O)(O)O)cc5c4)C(=O)N32)C[C@@H]1c1cc[nH]c(=O)c1 ; N#C[C@@H]1CN(C(=O)[C@@H]2CC[C@@H]3CCCC[C@H](NC(=O)c4ccc5ccc(C(F)(F)P(=O)(O)O)cc5c4)C(=O)N32)C[C@H]1c1cc[nH]c(=O)c1] | 682.2 [M + H]⁺ | ¹H NMR (400 MHz, DMSO-d₆) δ 8.70-8.57 (m, 2H), 8.20 (s, 1H), 8.15-7.99 (m, 3H), 7.78- 7.66 (m, 1H), 7.39 (d, J = 6.6 Hz, 1H), 6.43-6.20 (m, 2H), 5.09-4.95 (m, 1H), 4.61-4.52 (m, 1H), 4.37-4.21 (m, 1H), 3.98- 3.94 (m, 1H), 3.91- 3.87 (m, 1H), 3.83-3.79 (m, 1H), 3.71-3.67 (m, 1H), 3.61-3.56 (m, 1H), 3.52-3.44 (m, 1H), 3.36- 3.17 (m, 1H), 2.35- 1.99 (m, 3H), 1.97-1.74 (m, 6H), 1.70-1.50 (m, 3H) |
| | | SFC peak 2 was used for | | |
| | | biological testing | | |
| 81 | ((2- (((3S,6S,9aS)- 3-((2R,3S)-3-(1H- imidazol-1-yl)-2- methylazetidine-1- carbonyl)-5- oxooctahydro-1H- pyrrolo[1,2-a] azepin-6- yl)carbamoyl) benzo[b]thiophen-5- yl)difluoromethyl) phosphonic acid | 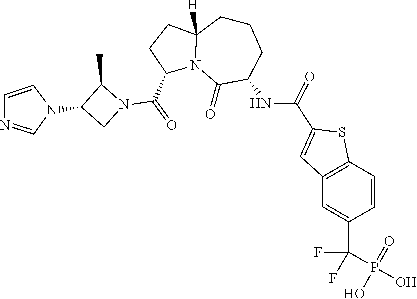 [SMILES: C[C@@H]1[C@@H](n2ccnc2)CN1C(=O)[C@@H]1CC[C@@H]2CCC[C@H](NC(=O)c3cc4cc(C(F)(F)P(=O)(O)O)ccc4s3)C(=O)N21] | 622.2 [M + H]⁺ | ¹H NMR (400 MHz, DMSO-d₆) δ 8.93-8.66 (m, 2H), 8.28 (s, 1H), 8.15-7.99 (m, 2H), 7.91- 7.74 (m, 1H), 7.58 (d, J = 8.4 Hz, 1H), 7.42-7.15 (m, 1H), 4.90-4.86 (m, 1H), 4.71-4.61 (m, 3H), 4.42-4.36 (m, 1H), 4.34- 4.28 (m, 1H), 4.08- 3.95 (m, 1H), 2.28-2.13 (m, 1H), 2.09-1.67 (m, 9H), 2.09-1.61 (m, 10H), 1.67-1.58 (m, 1H), 1.44 (d, J = 6.2 Hz, 2H) |
| 82 | (difluoro(2- (((3S,6S,10aS)-5- oxo-3-((S)-3- phenylpyrrolidine- 1-carbonyl) decahydropyrrolo [1,2-a]azocin-6- yl)carbamoyl) benzo[b]thiophen-5- yl)methyl) phosphonic acid | 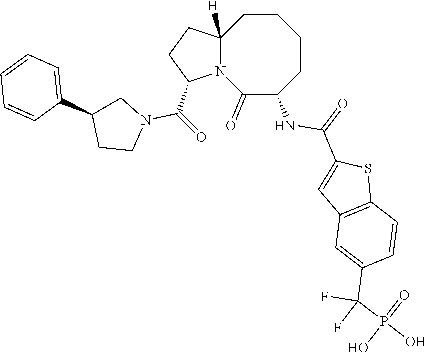 [SMILES: O=C(N[C@H]1CCCC[C@H]2CC[C@@H](C(=O)N3CC[C@@H](c4ccccc4)C3)N2C1=O)c1cc2cc(C(F)(F)P(=O)(O)O)ccc2s1] | 646.2 [M + H]⁺ | ¹H NMR (400 MHz, DMSO-d₆) δ 8.90-8.77 (m, 1H), 8.36-8.01 (m, 3H), 7.58 (d, J = 8.1 Hz, 1H), 7.38-6.90 (m, 5H), 5.02-4.90 (m, 1H), 4.63- 4.53 (m, 1H), 4.30- 4.23 (m, 1H), 3.80-3.72 (m, 2H), 3.60-3.53 (m, 1H), 3.43-3.21 (m, 2H), 2.36-2.16 (m, 2H), 2.15- 1.43 (m, 12H) |
| 83 | ((7- (((3S,6S,10aS)-3- (rel-(trans)-3- cyano-4- (2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphonic acid | 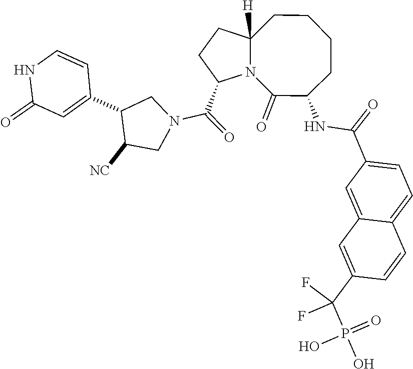 [SMILES: N#C[C@@H]1CN(C(=O)[C@@H]2CC[C@@H]3CCCC[C@H](NC(=O)c4ccc5ccc(C(F)(F)P(=O)(O)O)cc5c4)C(=O)N32)C[C@H]1c1cc[nH]c(=O)c1] | 682.3 [M + H]⁺ | ¹H NMR (400 MHz, DMSO-d₆) δ 8.77-8.57 (m, 2H), 8.20 (s, 1H), 8.13-7.95 (m, 3H), 7.72 (d, J = 8.3 Hz, 1H), 7.43- 7.32 (m, 1H), 6.45-6.15 (m, 2H), 5.08-4.96 (m, 1H), 4.60-4.49 (m, 1H), 4.39-4.29 (m, 1H), 4.19- 4.11 (m, 1H), 4.03- 3.98 (m, 1H), 3.92-3.88 (m, 1H), 3.85-3.78 (m, 1H), 3.67-3.59 (m, 1H), 3.53-3.42 (m, 1H), 2.35- 2.17 (m, 1H), 2.16- 2.02 (m, 1H), 2.01-1.75 (m, 7H), 1.71-1.49 (m, 3H) |
| 84 | ((2- (((3S,6S,10aS)- 3-(rel-(trans)- 3-cyano-4- phenylpyrrolidine- 1-carbonyl)-6- methyl-5- oxodecahydropyrrolo [1,2-a]azocin-6- yl)carbamoyl) benzo[b]thiophen-5- yl)difluoromethyl) phosphonic acid | 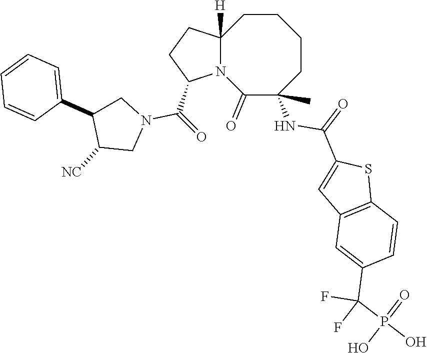 [SMILES: C[C@]1(NC(=O)c2cc3cc(C(F)(F)P(=O)(O)O)ccc3s2)CCCC[C@H]2CC[C@@H](C(=O)N3C[C@H](c4ccccc4)[C@@H](C#N)C3)N2C1=O] | 685.2 [M + H]⁺ | ¹H NMR (400 MHz, DMSO-d₆) δ 8.14 (m, 4H), 7.59 (m, 1H), 7.48 (m, 2H), 7.42 (m, 2H), 7.38-7.31 (m, 1H), 4.66- 4.60 (m, 1H), 4.59- 4.52 (m, 1H), 4.29 (m, 1H), 4.07 (m, 2H), 3.41- 3.33 (m, 2H), 3.35-3.27 (m, 1H), 2.85-2.77 (m, 1H), 2.30 (m, 2H), 2.06 (m, 3H), 1.76 (m, 2H), 1.73-1.68 (m, 3H), 1.50- 1.44 (m, 2H), 1.24 (s, 2H) |
| 85 | (difluoro(2- (((3S,6S,10aS)- 6-methyl-5-oxo-3- (3-(pyridin-2-yl) azetidine-1- carbonyl) decahydropyrrolo [1,2-a]azocin-6- yl)carbamoyl) benzo[b]thiophen- 5-yl)methyl) phosphonic acid | 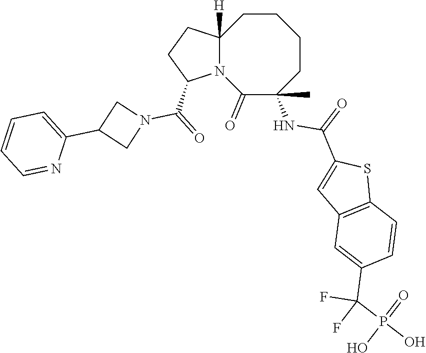 [SMILES: C[C@]1(NC(=O)c2cc3cc(C(F)(F)P(=O)(O)O)ccc3s2)CCCC[C@H]2CC[C@@H](C(=O)N3CC(c4ccccn4)C3)N2C1=O] | 647.0 [M + H]⁺ | ¹H NMR (400 MHz, DMSO-d₆) δ 8.68-8.58 (m, 1H), 8.27-8.05 (m, 4H), 7.93-7.80 (m, 1H), 7.64-7.31 (m, 3H), 4.79- 4.69 (m, 1H), 4.57- 4.53 (m, 1H), 4.36-4.31 (m, 2H), 4.26-4.12 (m, 3H), 2.88-2.78 (m, 1H), 2.35-2.27 (m, 1H), 2.22- 2.05 (m, 2H), 2.01- 1.92 (m, 1H), 1.81-1.71 (m, 2H), 1.68-1.60 (m, 5H), 1.52-1.41 (m, 2H), 1.30-1.19 (m, 1H) (TFA salt) |
| 86 | ((2- (((3S,6S,10aS)- 3-(rel-(trans)- 3-cyano-4- (2-oxo-1,2- dihydropyridin- 4-yl)pyrrolidine- 1-carbonyl)-5- oxodecahydropyrrolo [1,2-a]azocin-6- yl)carbamoyl) benzo[b]thiophen- 5-yl) difluoromethyl) phosphonic acid | 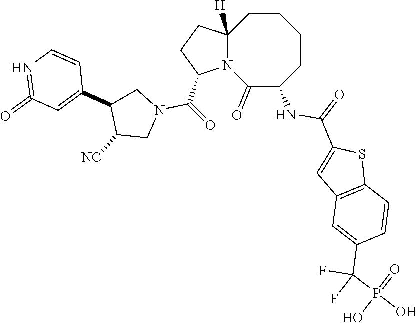 | 688.2 [M + H]⁺ | ¹H NMR (400 MHz, DMSO-d₆) δ 8.99-8.71 (m, 1H), 8.37-8.27 (m, 1H), 8.18-8.10 (m, 1H), 8.07 (s, 1H), 7.58 (d, J = 8.3 Hz, 1H), 7.44-7.31 (m, 1H), 6.43-6.09 (m, 2H), 5.02-4.87 (m, 1H), 4.64-4.52 (m, 1H), 4.33- 4.27 (m, 1H), 4.18- 4.12 (m, 1H), 4.04-3.96 (m, 1H), 3.94-3.87 (m, 1H), 3.85-3.77 (m, 1H), 3.65-3.54 (m, 1H), 3.44- 3.17 (m, 1H), 2.37- 2.15 (m, 1H), 2.13-1.44 (m, 11H) |
| 87 | ((2- (((3S,6S,10aS)-3- (4-(N,N- dimethylsulfamoyl) piperazine-1- carbonyl)-5- oxodecahydropyrrolo [1,2-a]azocin-6- yl)carbamoyl) benzo[b]thiophen- 5-yl) difluoromethyl) phosphonic acid | 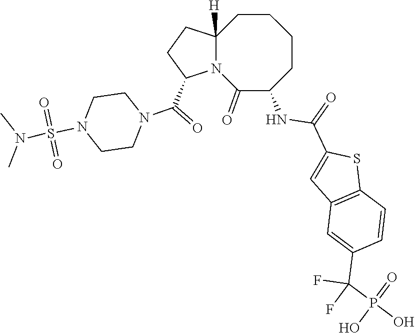 [SMILES: CN(C)S(=O)(=O)N1CCN(C(=O)[C@@H]2CC[C@@H]3CCCC[C@H](NC(=O)c4cc5cc(C(F)(F)P(=O)(O)O)ccc5s4)C(=O)N32)CC1] | 692.2 [M + H]⁺ | ¹H NMR (400 MHz, DMSO-d₆) δ 9.05-8.85 (m, 1H), 8.30 (s, 1H), 8.11-7.99 (m, 2H), 7.57 (d, J = 8.5 Hz, 1H), 4.97- 4.95 (m, 1H), 4.84-4.80 (m, 1H), 4.32-4.28 (m, 1H), 3.74-3.39 (m, 5H), 3.28-3.00 (m, 5H), 2.76 (s, 6H), 2.25-1.44 (m, 12H) |
| 88 | ((2- (((3S,6S,10aS)-3- (rel-(trans)- 3-cyano-4- ((tetrahydro- 2H-pyran-4- yl)methyl) pyrrolidine-1- carbonyl)-5- oxodecahydropyrrolo [1,2-a]azocin-6- yl)carbamoyl) benzo[b]thiophen- 5-yl)difluoromethyl) phosphonic acid | 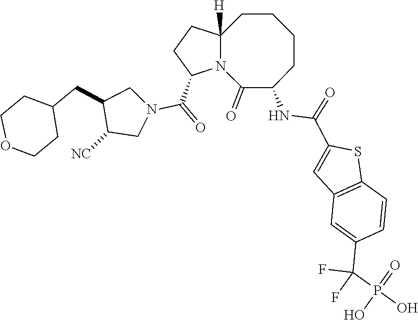 [SMILES: N#C[C@H]1CN(C(=O)[C@@H]2CC[C@@H]3CCCC[C@H](NC(=O)c4cc5cc(C(F)(F)P(=O)(O)O)ccc5s4)C(=O)N32)C[C@@H]1CC1CCOCC1] | 693.2 [M + H]⁺ | ¹H NMR (400 MHz, DMSO-d₆) δ 8.98-8.76 (m, 1H), 8.38-8.24 (m, 1H), 8.13 (d, J = 8.5 Hz, 1H), 8.06 (s, 1H), 7.58 (d, J = 8.6 Hz, 1H), 5.02- 4.82 (m, 1H), 4.62-2.62 (m, 14H), 2.37-0.98 (m, 17H) |
| 89 | (difluoro(2- (((3S,10aS)- 5-oxo-3-(6- phenyl-4- azaspiro[2.4] heptane-4- carbonyl) decahydropyrrolo [1,2-a]azocin-6- yl)carbamoyl) benzo[b]thiophen- 5-yl)methyl) phosphonic acid | 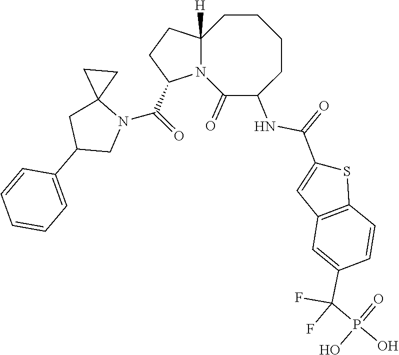 [SMILES: O=C(NC1CCCC[C@H]2CC[C@@H](C(=O)N3CC(c4ccccc4)CC34CC4)N2C1=O)c1cc2cc(C(F)(F)P(=O)(O)O)ccc2s1] | 672.3 [M + H]⁺ | ¹H NMR (400 MHz, DMSO-d₆) δ 8.86 (t, J = 7.4 Hz, 1H), 8.30 (d, J = 3.9 Hz, 1H), 8.15-7.94 (m, 2H), 7.58 (d, J = 8.2 Hz, 1H), 7.41-7.18 (m, 5H), 5.04-4.86 (m, 1H), 4.71-4.35 (m, 2H), 4.29- 4.17 (m, 1H), 4.16- 4.08 (m, 1H), 4.00-3.90 (m, 1H), 2.34-2.16 (m, 2H), 2.12-1.53 (m, 14H), 0.56-0.40 (m, 2H) |
| 90 | ((2- (((3S,6S,10aS)- 3-((3R,4S or 3S,4R)-3- cyano-4- phenylpyrrolidine- 1-carbonyl)-5- oxodecahydropyrrolo [1,2-a]azocin-6- yl)carbamoyl) benzo[b]thiophen- 5-yl)methyl) phosphonic acid | 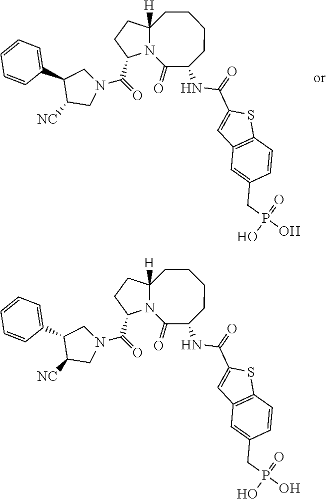 [SMILES: N#C[C@H]1CN(C(=O)[C@@H]2CC[C@@H]3CCCC[C@H](NC(=O)c4cc5cc(CP(=O)(O)O)ccc5s4)C(=O)N32)C[C@@H]1c1ccccc1 ; N#C[C@@H]1CN(C(=O)[C@@H]2CC[C@@H]3CCCC[C@H](NC(=O)c4cc5cc(CP(=O)(O)O)ccc5s4)C(=O)N32)C[C@H]1c1ccccc1] | 635.2 [M + H]⁺ | ¹H NMR (400 MHz, DMSO-d₆) δ 8.80-8.69 (m, 1H), 8.20 (d, J = 10.4 Hz, 1H), 7.95-7.86 (m, 1H), 7.79 (s, 1H), 7.56- 7.21 (m, 6H), 5.01-4.87 (m, 1H), 4.57-4.49 (m, 1H), 4.32-4.24 (m, 1H), 4.22-4.12 (m, 1H), 4.02- 3.95 (m, 1H), 3.83- 3.77 (m, 1H), 3.70-3.63 (m, 1H), 3.57-3.52 (m, 1H), 3.33-3.23 (m, 1H), 3.10 (d, J = 20.9 Hz, 2H), 2.32-2.19 (m, 1H), 2.14- 2.00 (m, 1H), 2.01- 1.68 (m, 7H), 1.69-1.43 (m, 3H) |
| | | SFC peak 2 was used for | | |
| | | biological testing | | |
| 91 | ((2- (((3S,6S,10aS)- 3-(rel-(trans)-3- cyano-4- cyclohexyl- pyrrolidine-1- carbonyl)-5- oxodecahydropyrrolo [1,2-a]azocin-6- yl)carbamoyl) benzo[b]thiophen- 5-yl) difluoromethyl) phosphonic acid | 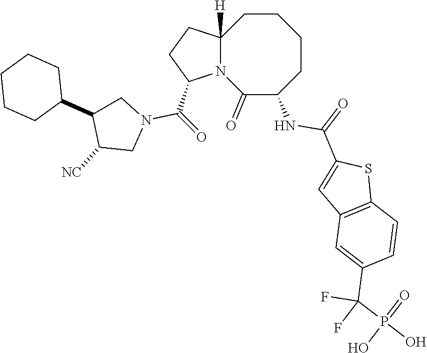 [SMILES: N#C[C@H]1CN(C(=O)[C@@H]2CC[C@@H]3CCCC[C@H](NC(=O)c4cc5cc(C(F)(F)P(=O)(O)O)ccc5s4)C(=O)N32)C[C@@H]1C1CCCCC1] | 677.2 [M + H]⁺ | ¹H NMR (400 MHz, DMSO-d₆) δ 8.96-8.78 (m, 1H), 8.37-8.27 (m, 1H), 8.13 (d, J = 8.5 Hz, 1H), 8.06 (s, 1H), 7.58 (d, J = 8.5 Hz, 1H), 5.02- 4.86 (m, 1H), 4.58-4.46 (m, 1H), 4.29-4.24 (m, 1H), 3.88-3.81 (m, 3H), 3.24-3.17 (m, 2H), 2.30- 2.16 (m, 2H), 1.97- 1.54 (m, 16H), 1.25-0.96 (m, 6H) |
| 92 | ((2- (((3S,6S,10aS)-3- (rel-(trans)- 3-cyano-4- phenylpyrrolidine- 1-carbonyl)-5- oxodecahydropyrrolo [1,2-a]azocin-6- yl)carbamoyl) benzo[b]thiophen- 5-yl) difluoromethyl) phosphonic acid | 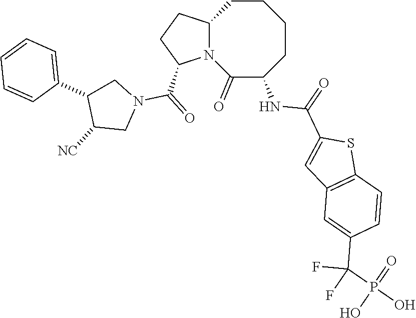 [SMILES: N#C[C@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1c1ccccc1] | 671.0 [M + H]⁺ | ¹H NMR (400 MHz, DMSO-d₆) δ 8.98-8.81 (m, 1H), 8.36-8.27 (m, 1H), 8.14-8.12 (m, 1H), 8.07 (s, 1H), 7.59-7.57 (m, 1H), 7.42-7.25 (m, 5H), 4.99-4.94 (m, 1H), 4.62-4.55 (m, 1H), 4.39- 4.28 (m, 2H), 4.15- 4.11 (m, 1H), 4.06-4.00 (m, 1H), 3.97-3.92 (m, 2H), 3.69-3.64 (m, 2H), 2.38-2.31 (m, 1H), 2.14- 2.07 (m, 2H), 1.91- 1.80 (m, 5H), 1.66-1.55 (m, 3H), 1.26-1.21 (m, 1H) |
| 93 | (difluoro(2- (((3S,6S,10aS)- 3-(3-(morpholine-4- carbonyl)azetidine-1- carbonyl)-5- oxodecahydropyrrolo [1,2-a]azocin-6- yl)carbamoyl) benzo[b]thiophen-5- yl)methyl) phosphonic acid | 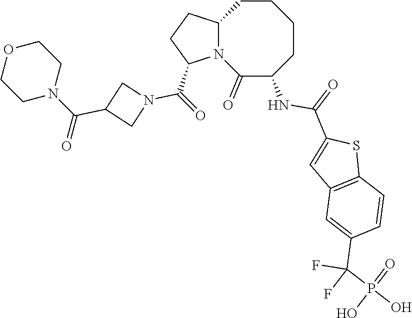 [SMILES: O=C(N[C@H]1CCCC[C@H]2CC[C@@H](C(=O)N3CC(C(=O)N4CCOCC4)C3)N2C1=O)c1cc2cc(C(F)(F)P(=O)(O)O)ccc2s1] | 669.3 [M + H]⁺ | ¹H NMR (400 MHz, DMSO-d₆) δ 8.87 (dd, J = 21.4, 6.9 Hz, 1H), 8.33 (s, 1H), 8.14 (d, J = 8.7 Hz, 1H), 8.07 (s, 1H), 7.59 (d, J = 8.5 Hz, 1H), 4.99- 4.89 (m, 2H), 4.64-4.40 (m, 4H), 4.29 (s, 1H), 4.10-3.85 (m, 6H), 3.78- 3.61 (m, 3H), 3.47- 3.39 (m, 2H), 2.14 (d, J = 7.6 Hz, 1H), 2.01 (d, J = 7.5 Hz, 1H), 1.94-1.75 (m, 6H), 1.75-1.44 (m, 4H) |
| 94 | (difluoro(2- (((3S,6S,9aS)-3- (3-(morpholine-4- carbonyl)azetidine- 1-carbonyl)-5- oxooctahydro-1H- pyrrolo[1,2-a] azepin-6- yl)carbamoyl) benzo[b]thiophen- 5-yl)methyl) phosphonic acid | 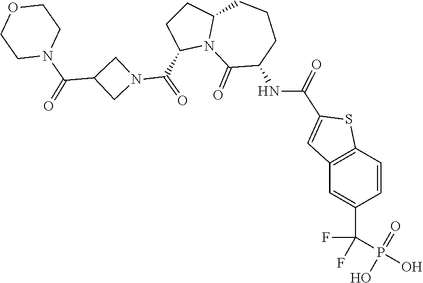 [SMILES: O=C(N[C@H]1CCC[C@H]2CC[C@@H](C(=O)N3CC(C(=O)N4CCOCC4)C3)N2C1=O)c1cc2cc(C(F)(F)P(=O)(O)O)ccc2s1] | 655.1 [M + H]⁺ | ¹H NMR (400 MHz, DMSO-d₆) δ 8.81-8.77 (m, 1H), 8.30 (s, 1H), 8.24-8.03 (m, 2H), 7.72- 7.45 (m, 1H), 4.72- 4.14 (m, 4H), 4.09-3.95 (m, 4H), 3.65-3.17 (m, 8H), 2.21-1.15 (m, 10H) |
| 95 | ((2- (((3S,6S,10aS)- 3-(rel-(trans)- 3-cyano-4- phenylpyrrolidine- 1-carbonyl)-5- oxodecahydropyrrolo [1,2-a]azocin-6- yl)carbamoyl) benzo[b]thiophen- 5-yl) difluoromethyl) phosphonic acid | 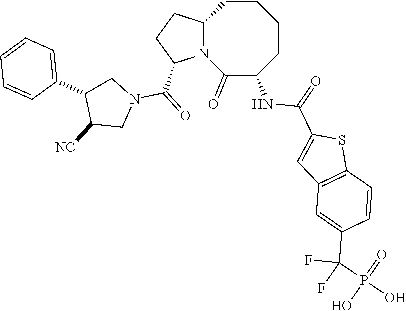 [SMILES: N#C[C@@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1c1ccccc1] | 671.3 [M + H]⁺ | ¹H NMR (400 MHz, DMSO-d₆) δ 8.89-8.79 (m, 1H), 8.36-8.25 (m, 1H), 8.13-8.03 (m, 2H), 7.60 (d, J = 9.1 Hz, 1H), 7.47-7.30 (m, 5H), 5.01- 4.89 (m, 1H), 4.63- 4.51 (m, 1H), 4.40-4.22 (m, 2H), 4.03-3.91 (m, 2H), 3.56-3.51 (m, 3H), 2.32-2.18 (m, 1H), 2.08- 1.75 (m, 8H), 1.68- 1.49 (m, 3H) |
| 96 | ((2- (((3S,6S,9aS)- 3-(rel-(trans)- 3-cyano-4- phenylpyrrolidine- 1-carbonyl)-5- oxooctahydro-1H- pyrrolo [1,2-a]azepin-6- yl)carbamoyl) benzo[b]thiophen- 5-yl)difluoromethyl) phosphonic acid | 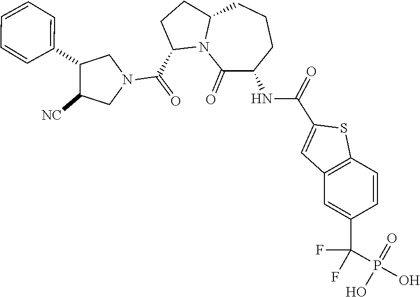 [SMILES: N#C[C@@H]1CN(C(=O)[C@@H]2CC[C@@H]3CCC[C@H](NC(=O)c4cc5cc(C(F)(F)P(=O)(O)O)ccc5s4)C(=O)N32)C[C@H]1c1ccccc1] | 657.3 [M + H]⁺ | ¹H NMR (400 MHz, DMSO-d₆) δ 8.81-8.70 (m, 1H), 8.31-8.25 (m, 1H), 8.12-8.03 (m, 2H), 7.59 (d, J = 8.4 Hz, 1H), 7.46-7.31 (m, 5H), 4.74- 4.58 (m, 2H), 4.03- 3.93 (m, 2H), 3.62-3.49 (m, 5H), 2.27-2.06 (m, 2H), 1.90-1.59 (m, 8H) |
| 97 | ((2- (((3S,6S,9aR)- 3-([1,1′- biphenyl]-4- ylcarbamoyl)-5- oxo-2,3,5,6,9,9a- hexahydro-1H- pyrrolo[1,2-a] azepin-6- yl)carbamoyl) benzo[b]thiophen- 5-yl) difluoromethyl) phosphonic acid | 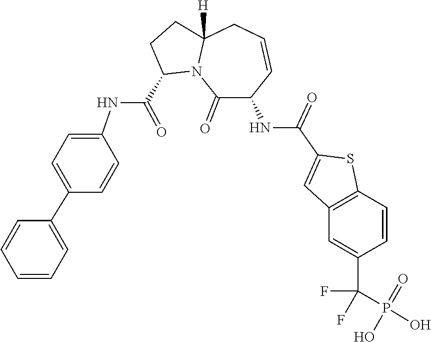 [SMILES: O=C(N[C@H]1C=CC[C@H]2CC[C@@H](C(=O)Nc3ccc(-c4ccccc4)cc3)N2C1=O)c1cc2cc(C(F)(F)P(=O)(O)O)ccc2s1] | 652.1 [M + H]⁺ | ¹H NMR (400 MHz, DMSO-d₆) δ 10.26 (s, 1H), 9.09 (d, J = 8.0 Hz, 1H), 8.32 (s, 1H), 8.19- 7.98 (m, 2H), 7.72-7.56 (m, 7H), 7.43 (t, J = 7.6 Hz, 2H), 7.32 (t, J = 7.3 Hz, 1H) 5.88-5.68 (m, 2H), 5.54-5.38 (m, 1H), 4.58-4.46 (m, 2H), 2.49- 1.71 (m, 6H) |
| 98 | (difluoro(2- (((3S,6S,10aS)- 3-((1-hydroxy-3- phenylpropan-2- yl)carbamoyl)-5- oxodecahydropyrrolo [1,2-a]azocin-6- yl)carbamoyl) benzo[b]thiophen- 5-yl)methyl) phosphonic acid | 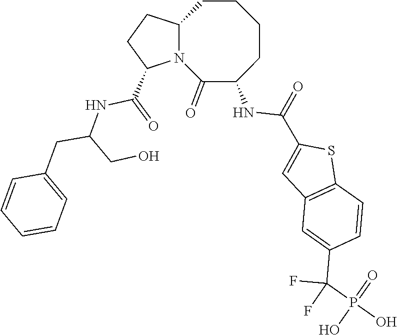 [SMILES: O=C(N[C@H]1CCCC[C@H]2CC[C@@H](C(=O)NC(CO)Cc3ccccc3)N2C1=O)c1cc2cc(C(F)(F)P(=O)(O)O)ccc2s1] | 650.2 [M + H]⁺ | ¹H NMR (400 MHz, DMSO-d₆) δ 9.25-8.88 (m, 1H), 8.54-8.50 (m, 1H), 8.35-8.30 (m, 1H), 8.11-8.04 (m, 1H), 7.95 (d, J = 7.1 Hz, 1H), 7.62 (d, J = 7.9 Hz, 1H), 7.45- 7.01 (m, 5H), 4.98-4.90 (m, 1H), 4.38-4.31 (m, 2H), 4.08-4.03 (m, 2H), 3.42-2.57 (m, 4H), 2.38- 1.37 (m, 12H) |
| 99 | ((2- (((3S,6S,10aS)-3- (rel-(trans)- 3-cyano-4- (1H-indazol-6- yl)pyrrolidine-1- carbonyl)-5- oxodecahydropyrrolo [1,2-a]azocin-6- yl)carbamoyl) benzo[b]thiophen-5- yl)difluoromethyl) phosphonic acid | 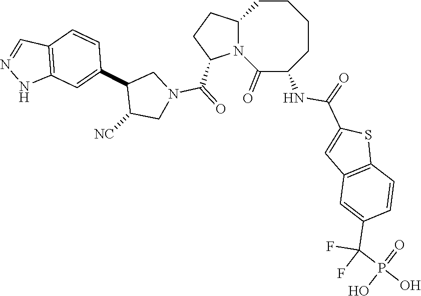 [SMILES: N#C[C@H]1CN(C(=O)[C@@H]2CC[C@@H]3CCCC[C@H](NC(=O)c4cc5cc(C(F)(F)P(=O)(O)O)ccc5s4)C(=O)N32)C[C@@H]1c1ccc2cn[nH]c2c1] | 711.3 [M + H]⁺ | ¹H NMR (400 MHz, DMSO-d₆) δ 13.04 (s, 1H), 9.31-8.76 (m, 1H), 8.39-8.27 (m, 1H), 8.19- 8.01 (m, 3H), 7.82- 7.70 (m, 1H), 7.62-7.41 (m, 2H), 7.25-7.05 (m, 1H), 5.05-4.91 (m, 1H), 4.80-4.47 (m, 2H), 4.42- 4.28 (m, 2H), 4.07- 3.95 (m, 4H), 2.36-2.06 (m, 2H), 2.03-1.74 (m, 7H), 1.70-1.47 (m, 3H) |
| 100 | (difluoro(2- (((3S,6S,8R,9R, 10aR)-9-hydroxy- 8-methyl-5- oxo-3-((S)-3- phenylpyrrolidine- 1-carbonyl) decahydropyrrolo [1,2-a]azocin-6- yl)carbamoyl) benzo[b]thiophen- 5-yl)methyl) phosphonic acid | 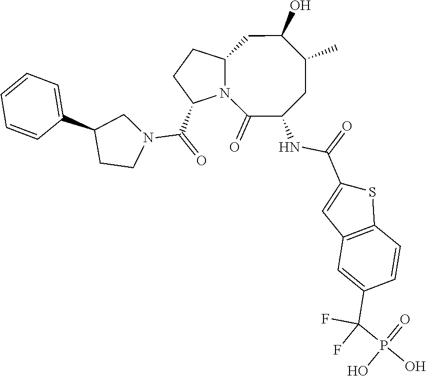 [SMILES: C[C@@H]1C[C@H](NC(=O)c2cc3cc(C(F)(F)P(=O)(O)O)ccc3s2)C(=O)N2[C@H](CC[C@H]2C(=O)N2CC[C@@H](c3ccccc3)C2)C[C@H]1O] | 676.0 [M + H]⁺ | ¹H NMR (400 MHz, DMSO-d₆) δ 8.92-8.78 (m, 1H), 8.34 (d, J = 1.9 Hz, 1H), 8.12 (d, J = 9.0 Hz, 1H), 8.07 (s, 1H), 7.59 (d, J = 8.6 Hz, 1H), 7.35-7.19 (m, 5H), 4.99- 4.88 (m, 1H), 4.59 (t, J = 8.2 Hz, 1H), 4.33-4.16 (m, 2H), 4.08-4.00 (m, 1H), 3.98-3.92 (m, 1H), 3.82-3.78 (m, 1H), 3.25- 3.17 (m, 2H), 2.25- 2.11 (m, 3H), 2.02-1.89 (m, 3H), 1.88-1.67 (m, 4H), 1.59-1.50 (m, 1H), 0.96 (d, J = 6.1 Hz, 3H) |
| 101 | (difluoro(2- (((3S,6S,8S,9S, 10aR)-8- hydroxy-9- methyl-5- oxo-3-((S)-3- phenylpyrrolidine- 1-carbonyl) decahydropyrrolo [1,2-a]azocin-6- yl)carbamoyl) benzo[b]thiophen- 5-yl)methyl) phosphonic acid | 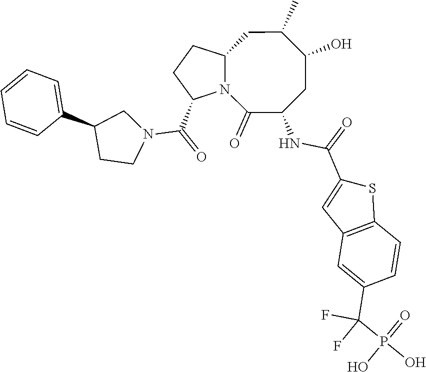 [SMILES: C[C@H]1C[C@H]2CC[C@@H](C(=O)N3CC[C@@H](c4ccccc4)C3)N2C(=O)[C@@H](NC(=O)c2cc3cc(C(F)(F)P(=O)(O)O)ccc3s2)C[C@H]1O] | 676.1 [M + H]⁺ | ¹H NMR (400 MHz, DMSO-d₆) δ 8.73-8.66 (m, 1H), 8.28 (s, 1H), 8.05 (s, 2H), 7.60 (d, J = 8.8 Hz, 1H), 7.36-7.22 (m, 5H), 5.00-4.92 (m, 1H), 4.62-4.58 (m, 1H), 4.45-4.35 (m, 1H), 4.26- 3.95 (m, 1H), 3.82- 3.77 (m, 1H), 3.59-3.53 (m, 2H), 3.18-3.17 (m, 2H), 2.23-2.14 (m, 3H), 1.95-1.83 (m, 3H), 1.76- 1.61 (m, 3H), 1.56- 1.50 (m, 1H), 1.47-1.44 (m, 1H), 0.98 (d, J = 4.7 Hz, 3H) |
| 102 | ((2- (((3S,6S,10aS)-3- (rel-(trans)- 3-cyano-4- cyclopropyl- pyrrolidine-1- carbonyl)-5- oxodecahydropyrrolo [1,2-a]azocin-6- yl)carbamoyl) benzo[b]thiophen- 5-yl) difluoromethyl) phosphonic acid | 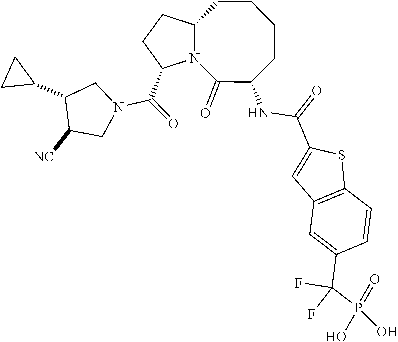 [SMILES: N#C[C@@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1C1CC1] | 635.1 [M + H]⁺ | ¹H NMR (400 MHz, DMSO-d₆) δ 8.92-8.80 (m, 1H), 8.33-8.30 (m, 1H), 8.14 (d, J = 8.5 Hz, 1H), 8.07 (s, 1H), 7.58 (d, J = 8.6 Hz, 1H), 5.00- 4.90 (m, 1H), 4.57-4.48 (m, 1H), 4.30-4.25 (m, 1H), 3.81-3.61 (m, 2H), 3.55-3.40 (m, 2H), 2.33- 2.21 (m, 1H), 2.06- 1.52 (m, 12H), 1.28-1.21 (m, 1H), 0.87-0.78 (m, 1H), 0.57-0.48 (m, 2H), 0.32-0.24 (m, 2H) |
| 103 | (difluoro(2- (((3S,6S,10aR)- 10-methyl-5- oxo-3-(3- (pyridin-3- yl)azetidine-1- carbonyl) decahydropyrrolo [1,2-a]azocin-6- yl)carbamoyl) benzo[b]thiophen-5- yl)methyl) phosphonic acid | 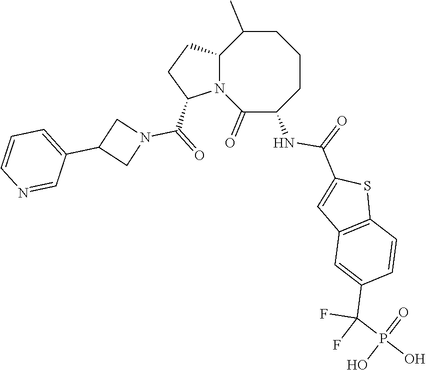 [SMILES: CC1CCC[C@H](NC(=O)c2cc3cc(C(F)(F)P(=O)(O)O)ccc3s2)C(=O)N2[C@@H]1CC[C@H]2C(=O)N1CC(c2cccnc2)C1] | 647.2 [M + H]⁺ | ¹H NMR (400 MHz, DMSO-d₆) δ 8.91-8.77 (m, 1H), 8.59 (s, 1H), 8.53-8.40 (m, 1H), 8.31 (s, 1H), 8.16-8.05 (m, 2H), 8.01-7.89 (m, 1H), 7.59 (d, J = 8.5 Hz, 1H), 7.51-7.30 (m, 1H), 4.86- 4.63 (m, 2H), 4.62- 4.55 (m, 1H), 4.45-4.39 (m, 1H), 4.30-4.27 (m, 1H), 4.21-4.17 (m, 1H), 3.95-3.83 (m, 2H), 2.27- 2.13 (m, 1H), 2.06- 1.93 (m, 3H), 1.92-1.39 (m, 7H), 0.93-0.83 (m, 3H) |
| 104 | ((2- (((3S,6S,10aR)-3- ((3R,4S or 3S,4R)-3- cyano-4-(2- oxo-1,2- dihydropyridin-4- yl)pyrrolidine-1- carbonyl)- 10-methyl-5- oxodecahydropyrrolo [1,2-a]azocin-6- yl)carbamoyl) benzo[b]thiophen-5- yl) difluoromethyl) phosphonic acid | 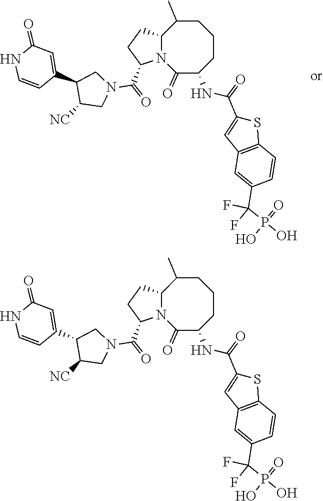 [SMILES: CC1CCC[C@H](NC(=O)c2cc3cc(C(F)(F)P(=O)(O)O)ccc3s2)C(=O)N2[C@@H]1CC[C@H]2C(=O)N1C[C@H](c2cc[nH]c(=O)c2)[C@@H](C#N)C1 ; CC1CCC[C@H](NC(=O)c2cc3cc(C(F)(F)P(=O)(O)O)ccc3s2)C(=O)N2[C@@H]1CC[C@H]2C(=O)N1C[C@@H](C#N)[C@H](c2cc[nH]c(=O)c2)C1] | 702.2 [M + H]⁺ | ¹H NMR (400 MHz, DMSO-d₆) δ 8.88-8.64 (m, 1H), 8.29 (d, J = 6.0 Hz, 1H), 8.17-8.01 (m, 2H), 7.66-7.49 (m, 1H), 7.45-7.29 (m, 1H), 6.47- 6.32 (m, 1H), 6.32- 6.18 (m, 1H), 4.75-4.58 (m, 1H), 4.53-4.38 (m, 1H), 4.23-4.12 (m, 1H), 4.09-3.99 (m, 1H), 3.95- 3.88 (m, 1H), 3.87- 3.82 (m, 1H), 3.80-3.76 (m, 1H), 3.17 (dd, J = 25.0, 10.2 Hz, 2H), 2.28- 1.91 (m, 5H), 1.88-1.47 (m, 7H), 1.36-1.18 (m, 1H), 0.99-0.75 (m, 3H) |
| | | SFC peak 2 was used for | | |
| | | biological testing | | |
| 105 | ((2- (((3S,6S,10aS)- 3-(rel-(trans) 3-cyano-4-(6- methoxypyridin-2- yl)pyrrolidine-1- carbonyl)-5- oxodecahydropyrrolo [1,2-a]azocin-6- yl)carbamoyl) benzo[b]thiophen-5- yl)difluoromethyl) phosphonic acid | 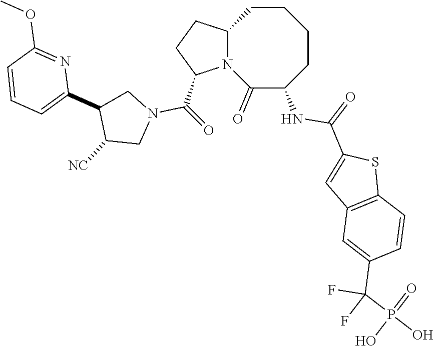 [SMILES: COc1cccc([C@H]2CN(C(=O)[C@@H]3CC[C@@H]4CCCC[C@H](NC(=O)c5cc6cc(C(F)(F)P(=O)(O)O)ccc6s5)C(=O)N43)C[C@@H]2C#N)n1] | 702.1 [M + H]⁺ | ¹H NMR (400 MHz, DMSO-d₆) δ 8.93-8.72 (m, 2H), 8.41-8.24 (m, 1H), 8.20-7.98 (m, 2H), 7.79-7.66 (m, 1H), 7.58 (d, J = 8.4 Hz, 1H), 7.11- 6.96 (m, 1H), 6.82-6.66 (m, 1H), 5.02-4.88 (m, 1H), 4.62-4.48 (m, 1H), 4.45-4.22 (m, 3H), 4.17- 3.91 (m, 3H), 3.88- 3.80 (m, 3H), 3.75-3.62 (m, 1H), 3.56-3.38 (m, 1H), 2.36-2.18 (m, 1H), 2.16-2.01 (m, 1H), 2.00- 1.70 (m, 7H), 1.70- 1.44 (m, 3H) |
| 106 | ((7- (((1R,3S,6S, 11aS)-3- ((3S,4R or 3R,4S)-3- cyano-4-(2- oxo-1,2- dihydropyridin-4- yl)pyrrolidine-1- carbonyl)-1- hydroxy-5- oxodecahydro-1H- pyrrolo[1,2-a] azonin-6-yl) carbamoyl) naphthalen-2- yl)difluoromethyl) phosphonic acid | 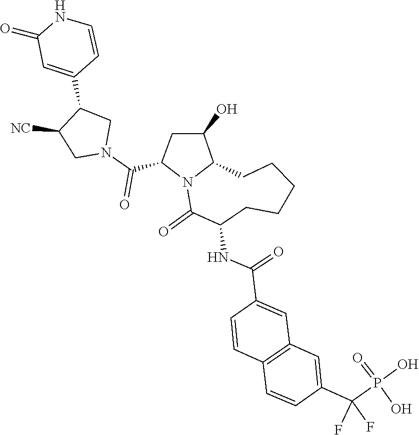 [SMILES: N#C[C@@H]1CN(C(=O)[C@@H]2C[C@@H](O)[C@@H]3CCCCC[C@H](NC(=O)c4ccc5ccc(C(F)(F)P(=O)(O)O)cc5c4)C(=O)N23)C[C@H]1c1cc[nH]c(=O)c1] | 712.1 [M + H]⁺ | ¹H NMR (400 MHz, DMSO-d₆) δ 8.68-8.58 (m, 2H), 8.20-8.19 (m, 1H), 8.09-8.04 (m, 4H), 7.72-7.70 (m,, 1H), 7.40- 7.37 (m, 1H), 6.38- 6.36 (m, 1H), 6.29-6.22 (m, 1H), 5.20-5.13 (m, 1H), 4.70-4.66 (m, 1H), 4.14-4.10 (m, 1H), 4.07- 4.04 (m, 1H), 3.99- 3.97 (m, 1H), 3.82-3.78 (m, 1H), 3.67-3.66 (m, 1H), 3.63-3.61 (m, 1H), 3.50-3.47 (m, 1H), 3.24- 3.19 (m, 1H), 2.16- 1.94 (m, 4H), 1.88-1.85 (m, 3H), 1.65 (m, 3H), 1.58-1.53 (m, 2H), 1.44- 1.40 (m, 1H) |
| | | or | | |
| | | 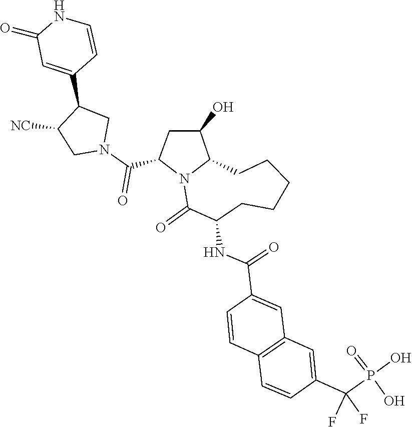 [SMILES: N#C[C@H]1CN(C(=O)[C@@H]2C[C@@H](O)[C@@H]3CCCCC[C@H](NC(=O)c4ccc5ccc(C(F)(F)P(=O)(O)O)cc5c4)C(=O)N23)C[C@@H]1c1cc[nH]c(=O)c1] | | |
| | | SFC peak 2 was used for | | |
| | | biological testing | | |
| 107 | ((2- (((4S,7S,10aS)- 4-((3S,4R or 3R,4S)-3- cyano-4- phenylpyrrolidine-1- carbonyl)-6- oxodecahydropyrido [1,2-a]azepin-7- yl)carbamoyl) benzo[b]thiophen- 5-yl) difluoromethyl) phosphonic acid | 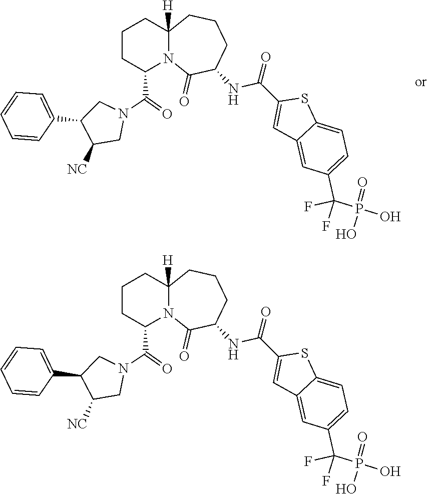 [SMILES: N#C[C@@H]1CN(C(=O)[C@@H]2CCC[C@@H]3CCC[C@H](NC(=O)c4cc5cc(C(F)(F)P(=O)(O)O)ccc5s4)C(=O)N32)C[C@H]1c1ccccc1 ; N#C[C@H]1CN(C(=O)[C@@H]2CCC[C@@H]3CCC[C@H](NC(=O)c4cc5cc(C(F)(F)P(=O)(O)O)ccc5s4)C(=O)N32)C[C@@H]1c1ccccc1] | 669.0 [M − H]⁻ | ¹H NMR (600 MHz, DMSO-d₆) δ 8.85-8.71 (m, 1H), 8.34-8.24 (m, 1H), 8.12 (dd, J = 8.7, 6.0 Hz, 1H), 8.06 (s, 1H), 7.63-7.53 (m, 1H), 7.46- 7.35 (m, 3H), 7.31 (t, J = 7.7 Hz, 1H), 5.02 (d, J = 8.9 Hz, 1H), 4.88 (ddt, J = 14.0, 11.0, 5.2 Hz, 1H), 4.14-4.04 (m, 2H), 3.95 (ddd, J = 18.2, 11.2, 7.3 Hz, 2H), 3.69-3.66 (m, 2H), 3.56-3.43 (m, 3H), 3.23 (dt, J = 30.9, 10.8 Hz, 1H), 2.39 (s, 1H), 1.99- 1.47 (m, 9H), 1.47-1.32 (m, 2H) |
| | | SFC peak 2 was used for | | |
| | | biological testing | | |
| 108 | (difluoro(2- (((3S,6S,9aS)-5- oxo-3-(3- (pyridin-2-yl) azetidine-1- carbonyl)octahydro- 1H-pyrrolo[1,2- a]azepin-6- yl)carbamoyl) benzo[b]thiophen- 5-yl)methyl) phosphonic acid | 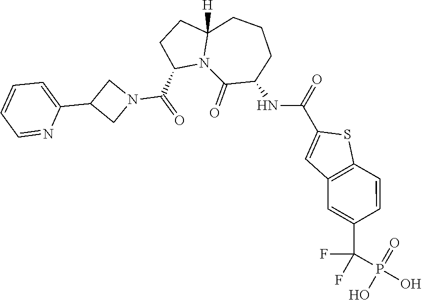 [SMILES: O=C(N[C@H]1CCC[C@H]2CC[C@@H](C(=O)N3CC(c4ccccn4)C3)N2C1=O)c1cc2cc(C(F)(F)P(=O)(O)O)ccc2s1] | 618.9 [M + H]⁺ | ¹H NMR (400 MHz, methanol-d₄) δ 8.75- 8.55 (m, 1H), 8.43-8.22 (m, 1H), 8.21-8.15 (m, 1H), 8.13 (d, J = 2.8 Hz, 1H), 8.05-7.96 (m, 1H), 7.90-7.85 (m, 1H), 7.80- 7.65 (m, 1H), 7.58- 7.37 (m, 1H), 4.79-4.69 (m, 2H), 4.61-4.39 (m, 2H), 4.30-4.00 (m, 3H), 3.53-3.45 (m, 1H), 2.25- 2.45 (m, 1 H), 2.21- 1.74 (m, 8H), 1.40-1.19 (m, 1H) |

### Synthesis of (difluoro(2-(((3S,6S,8R,9aR)-8-methyl-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphonic acid or (difluoro(2-(((3S,6S,8S,9aR)-8-methyl-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphonic acid (109/110)

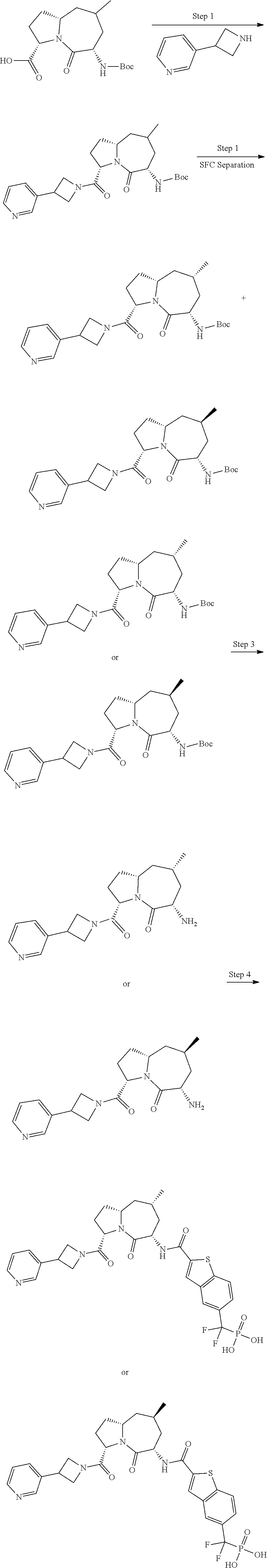

### Step 1: Preparation of Tert-Butyl ((3S,6S,9aR)-8-methyl-5-oxo-3-(3-(pyridin-3-yl)azetidine-1-carbonyl)octahydro-1H-pyrrolo[1,2-a]azepin-6-yl)carbamate

To a solution of 3-(azetidin-3-yl)pyridine (0.16 g, 1.2 mmol, 1 eq) and N,N-diisopropylethylamine (0.47 g, 3.7 mmol, 3.0 eq) in DMF (1.0 mL) was sequentially added a solution of (3S,6S,9aR)-6-{[(tert-butoxy)carbonyl]amino}-8-methyl-5-oxo-octahydro-1Hpyrrolo[1,2-a]azepine-3-carboxylic acid (0.40 g, 1.2 mmol, 1 eq) in DMF (1.0 mL). To this mixture was introduced HATU (0.7 g, 1.8 mmol, 1.5 eq), N,N-diisopropylethylamine (0.47 g, 3.7 mmol, 3.0 eq). The yellow solution was stirred for 1 h, and subsequently diluted with water (50 mL). The aqueous phase was extracted with EtOAc (50 mL×2). The combined organic layers were washed with saturated aqueous brine (50 mL×2), dried over Na₂SO₄, filtered and concentrated in vacuo. The residue was purified by column chromatography (elution with CH₂Cl₂/MeOH) to give tert-butyl ((3S,6S,9aR)-8-methyl-5-oxo-3-(3-(pyridin-3-yl)azetidine-1-carbonyl)octahydro-1H-pyrrolo[1,2-a]azepin-6-yl)carbamate (0.29 mg, 53% yield) as a yellow oil. LCMS (ESI) m/z=443.1 [M+H]⁺.

### Step 2: Preparation of Tert-Butyl ((3S,6S,8R,9aR)-8-methyl-5-oxo-3-(3-(pyridin-3-yl)azetidine-1-carbonyl)octahydro-1H-pyrrolo[1,2-a]azepin-6-yl)carbamate and tert-butyl ((3S,6S,8S,9aR)-8-methyl-5-oxo-3-(3-(pyridin-3-yl)azetidine-1-carbonyl)octahydro-1H-pyrrolo[1,2-a]azepin-6-yl)carbamate

tert-Butyl ((3S,6S,9aR)-8-methyl-5-oxo-3-(3-(pyridin-3-yl)azetidine-1-carbonyl)octahydro-1H-pyrrolo[1,2-a]azepin-6-yl)carbamate (0.29 mg) was purified by SFC (Column: DAICEL CHIRALPAK AD 250 mm×30 mm, 10 um, Mobile phase: Phase A for CO₂, and Phase B for Neu-IPA; Gradient elution: B in A 30%) to yield

Peak 1: tert-butyl ((3S,6S,8R,9aR)-8-methyl-5-oxo-3-(3-(pyridin-3-yl)azetidine-1-carbonyl)octahydro-1H-pyrrolo[1,2-a]azepin-6-yl)carbamate or tert-butyl ((3S,6S,8S,9aR)-8-methyl-5-oxo-3-(3-(pyridin-3-yl)azetidine-1-carbonyl)octahydro-1H-pyrrolo[1,2-a]azepin-6-yl)carbamate (0.14 g, 48% yield, tR=1.536 min) as a white solid. LCMS (ESI) m/z=443.1 [M+H]⁺.

Peak 2: tert-butyl ((3S,6S,8S,9aR)-8-methyl-5-oxo-3-(3-(pyridin-3-yl)azetidine-1-carbonyl)octahydro-1H-pyrrolo[1,2-a]azepin-6-yl)carbamate or tert-butyl ((3S,6S,8R,9aR)-8-methyl-5-oxo-3-(3-(pyridin-3-yl)azetidine-1-carbonyl)octahydro-1H-pyrrolo[1,2-a]azepin-6-yl)carbamate (80 mg, 28% yield, tR=1.662 min) as a white solid. LCMS (ESI) m/z=443.1 [M+H]⁺.

### Step 3: Preparation of (3S,6S,8R,9aR)-6-amino-8-methyl-3-(3-(pyridin-3-yl)azetidine-1-carbonyl)octahydro-5H-pyrrolo[1,2-a]azepin-5-one or (3S,6S,8S,9aR)-6-amino-8-methyl-3-(3-(pyridin-3-yl)azetidine-1-carbonyl)octahydro-5H-pyrrolo[1,2-a]azepin-5-one

To a solution of tert-butyl ((3S,6S,8R,9aR)-8-methyl-5-oxo-3-(3-(pyridin-3-yl)azetidine-1-carbonyl)octahydro-1H-pyrrolo[1,2-a]azepin-6-yl)carbamate or tert-butyl ((3S,6S,8S,9aR)-8-methyl-5-oxo-3-(3-(pyridin-3-yl)azetidine-1-carbonyl)octahydro-1H-pyrrolo[1,2-a]azepin-6-yl)carbamate [Peak 1] (1 eq) in CH₂Cl₂ (0.3 mL) was added trifluoroacetic acid (0.1 mL). The resulting yellow reaction mixture was stirred for 15 min and subsequently concentrated in vacuo and dried to give (3S,6S,8R,9aR)-6-amino-8-methyl-3-(3-(pyridin-3-yl)azetidine-1-carbonyl)octahydro-5H-pyrrolo[1,2-a]azepin-5-one or (3S,6S,8S,9aR)-6-amino-8-methyl-3-(3-(pyridin-3-yl)azetidine-1-carbonyl)octahydro-5H-pyrrolo[1,2-a]azepin-5-one (140 mg, TFA salt) as a yellow oil. LCMS (ESI) m/z=343.1 [M+H]⁺.

The intermediate in Table 29 was prepared according to the method described above starting from tert-butyl ((3S,6S,8S,9aR)-8-methyl-5-oxo-3-(3-(pyridin-3-yl)azetidine-1-carbonyl)octahydro-1H-pyrrolo[1,2-a]azepin-6-yl)carbamate or tert-butyl ((3S,6S,8R,9aR)-8-methyl-5-oxo-3-(3-(pyridin-3-yl)azetidine-1-carbonyl)octahydro-1H-pyrrolo[1,2-a]azepin-6-yl)carbamate [Peak 2] and using the appropriate conditions.

TABLE 29

| Name | Structure | LCMS |
|---|---|---|
| (3S,6S,8S,9aR)-6- amino-8-methyl-3- (3-(pyridin-3-yl) azetidine-1-carbonyl) octahydro-5H-pyrrolo [1,2-a]azepin-5-one or (3S,6S,8S,9aR)-6- amino-8-methyl-3- (3-(pyridin-3-yl) azetidine-1-carbonyl) octahydro-5H-pyrrolo [1,2-a]azepin-5-one | 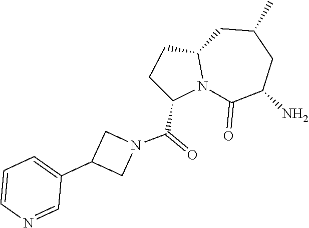 [SMILES: C[C@H]1C[C@H]2CC[C@@H](C(=O)N3CC(c4cccnc4)C3)N2C(=O)[C@@H](N)C1] | 343.1 [M + H]⁺ |
| or | or | |
| (3S,6S,8R,9aR)-6- amino-8-methyl-3- (3-(pyridin-3-yl) azetidine-1-carbonyl) octahydro-5H-pyrrolo [1,2-a]azepin-5-one or (3S,6S,8S,9aR)-6- amino-8-methyl-3- (3-(pyridin-3- yl)azetidine-1-carbonyl)octahydro- 5H-pyrrolo[1,2-a] azepin-5-one | 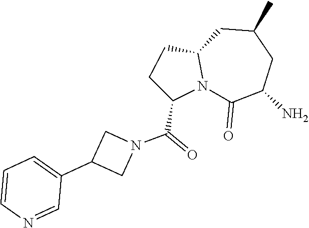 [SMILES: C[C@@H]1C[C@H]2CC[C@@H](C(=O)N3CC(c4cccnc4)C3)N2C(=O)[C@@H](N)C1] | |

### Step 4: Preparation of (difluoro(2-(((3S,6S,8R,9aR)-8-methyl-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphonic acid or (difluoro(2-(((3S,6S,8S,9aR)-8-methyl-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphonic acid (109/110)

To a solution of (difluoro(2-((4-nitrophenoxy)carbonyl)benzo[b]thiophen-5-yl)methyl)phosphonic acid (1 eq) in DMF (1 mL) was added HOBt (1.5 eq) and N,N-diisopropylethylamine (3 eq). After stirring for 30 min, the solution of (3S,6S,8R,9aR)-6-amino-8-methyl-3-(3-(pyridin-3-yl)azetidine-1-carbonyl)octahydro-5H-pyrrolo[1,2-a]azepin-5-one or (3S,6S,8S,9aR)-6-amino-8-methyl-3-(3-(pyridin-3-yl)azetidine-1-carbonyl)octahydro-5H-pyrrolo[1,2-a]azepin-5-one [from Step 3 and derived from Peak 1] (1.2 eq) in DMF (1 mL) and N,N-diisopropylethylamine (3 eq) was added to the reaction mixture. The resulting yellow reaction mixture was stirred for 30 min, and subsequently purified by prep-HPLC (column: Phenomenex Luna C18 150×25 mm×10 um, mobile phase: water (NH₄HCO₃)-acetonitrile; B %: 4%-34%, 8 min) to give (difluoro(2-(((3S,6S,8R,9aR)-8-methyl-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphonic acid or (difluoro(2-(((3S,6S,8S,9aR)-8-methyl-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphonic acid (109) (21 mg, 14% yield). LCMS (ESI) m/z=633.3 [M+H]⁺; ¹H NMR (400 MHz, methanol-d₄) δ 8.69-8.40 (m, 2H), 8.22-8.06 (m, 3H), 7.94 (d, J=8.4 Hz, 1H), 7.75 (d, J=8.0 Hz, 1H), 7.48 (s, 1H), 4.77 (d, J=10.0 Hz, 1H), 4.69 (s, 1H), 4.62-4.47 (m, 2H), 4.45-4.24 (m, 1H), 4.14-3.93 (m, 3H), 2.31 (s, 1H), 2.18-2.03 (m, 2H), 1.96 (d, J=9.6 Hz, 3H), 1.86-1.72 (m, 1H), 1.70-1.52 (m, 2H), 1.05 (s, 3H).

The compound in Table 30 was prepared according to the method described above starting from (3S,6S,8S,9aR)-6-amino-8-methyl-3-(3-(pyridin-3-yl)azetidine-1-carbonyl)octahydro-5H-pyrrolo[1,2-a]azepin-5-one or (3S,6S,8R,9aR)-6-amino-8-methyl-3-(3-(pyridin-3-yl)azetidine-1-carbonyl)octahydro-5H-pyrrolo[1,2-a]azepin-5-one [derived from Peak 2] and using the appropriate conditions.

TABLE 30

| Compound | Name | Structure | LCMS | ¹H NMR |
|---|---|---|---|---|
| 110 | (difluoro(2- (((3S,6S,8R,9aR)- 8-methyl-5-oxo- 3-(3-(pyridin-3- yl)azetidine-1- carbonyl) octahydro- 1H-pyrrolo[1,2- a]azepin-6- yl)carbamoyl) benzo[b]thiophen- 5-yl)methyl) phosphonic acid | 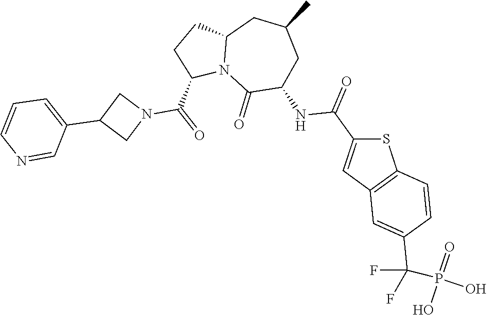 [SMILES: C[C@@H]1C[C@H]2CC[C@@H](C(=O)N3CC(c4cccnc4)C3)N2C(=O)[C@@H](NC(=O)c2cc3cc(C(F)(F)P(=O)(O)O)ccc3s2)C1] | 633.1 [M + H]⁺ | ¹H NMR (400 MHZ, methanol-d₄) δ 8.54 (d, J = 8.0 Hz, 1H), 8.47-8.42 (m, 1H), 8.28-8.10 (m, 3H), 7.95-7.90 (m, 1H), 7.85-7.78 (m, 1H), 7.50-7.42 (m, 1H), 5.02-4.96 (m, 2H), 4.73-4.66 (m, 1H), 4.64-4.58 (m, 1H), 4.56-4.51 (m, 1H), 4.32-4.26 (m, 1H), 4.08-3.96 (m, 2H), 2.38-2.27 (m, 2H), 2.21-2.00 (m, 6H), 1.98-1.81 (m, 2H), 1.65-1.55(m, 2H) |
| | or | or | | |
| | (difluoro(2- (((3S,6S,8S,9aR)- 8-methyl-5-oxo- 3-(3-(pyridin-3- yl)azetidine-1- carbonyl) octahydro- 1H-pyrrolo[1,2- a]azepin-6- yl)carbamoyl) benzo[b]thiophen- 5-yl)methyl) phosphonic acid | 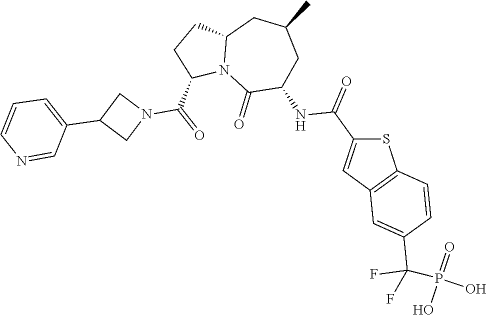 [SMILES: C[C@@H]1C[C@H]2CC[C@@H](C(=O)N3CC(c4cccnc4)C3)N2C(=O)[C@@H](NC(=O)c2cc3cc(C(F)(F)P(=O)(O)O)ccc3s2)C1] | | |

Method 3: Representative Procedure for the Synthesis of Phosphonic Acid Analogues via Amino Acid Coupling of Core Carboxylic Acids with Amines

### Synthesis of (Difluoro(2-(((3S,6S,9aS)-3-(methyl(phenyl)carbamoyl)-5-oxooctahydro-1H-pyrrolo[1,2-a]azepin-6-yl)carbamoyl)benzo[b]thiophen-5-yl)methyl)phosphonic acid (111)

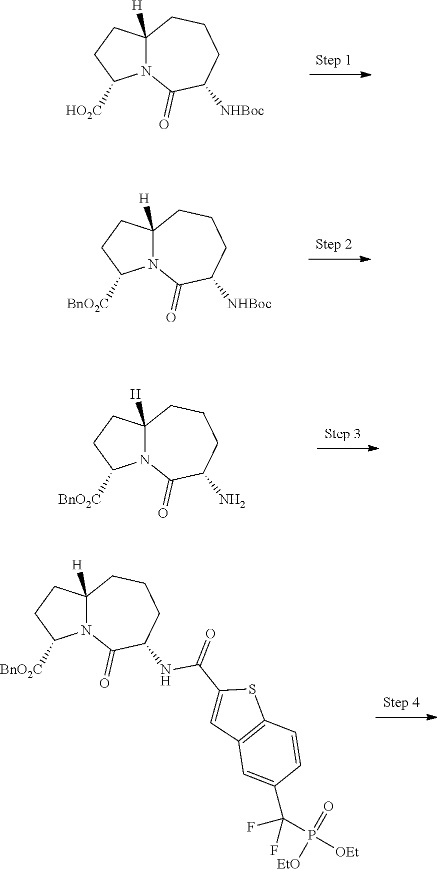

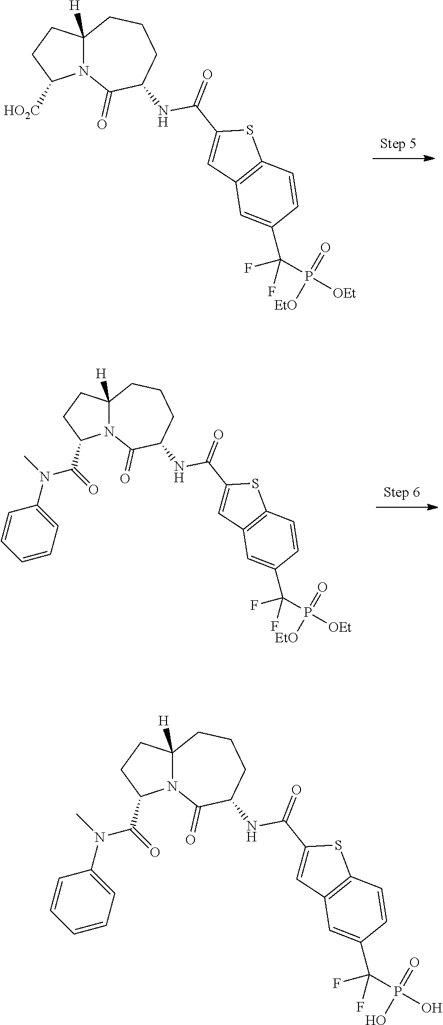 [SMILES: CCOP(=O)(OCC)C(F)(F)c1ccc2sc(C(=O)N[C@H]3CCC[C@H]4CC[C@@H](C(=O)O)N4C3=O)cc2c1 ; CCOP(=O)(OCC)C(F)(F)c1ccc2sc(C(=O)N[C@H]3CCC[C@H]4CC[C@@H](C(=O)N(C)c5ccccc5)N4C3=O)cc2c1 ; CN(C(=O)[C@@H]1CC[C@@H]2CCC[C@H](NC(=O)c3cc4cc(C(F)(F)P(=O)(O)O)ccc4s3)C(=O)N21)c1ccccc1]

### Step 1: Preparation of Benzyl (3S,6S,9aS)-6-((tert-butoxycarbonyl)amino)-5-oxooctahydro-1H-pyrrolo[1,2-a]azepine-3-carboxylate

To a solution of (3S,6S,9aS)-6-((tert-butoxycarbonyl)amino)-5-oxooctahydro-1H-pyrrolo[1,2-a]azepine-3-carboxylic acid (590 mg, 1.88 mmol, 1 eq) and Cs₂CO₃ (1.22 g, 3.76 mmol, 2 eq) in THF (15 mL) was added benzyl bromide (352 mg, 2.06 mmol, 1.1 eq). The resulting mixture was stirred for an additional 12 h at room temperature, followed by addition of H₂O (10 mL). The resulting suspension was extracted with CH₂Cl₂ (10 mL×3). The combined organic layers were washed with brine (20 mL), dried over anhydrous Na₂SO₄, and concentrated under reduced pressure. The residue was purified by flash column chromatography to yield benzyl (3S,6S,9aS)-6-((tert-butoxycarbonyl)amino)-5-oxooctahydro-1H-pyrrolo[1,2-a]azepine-3-carboxylate (633 mg, 1.57 mmol, 84% yield) as a white solid. LCMS (ESI) m/z=403 [M+H]⁺.

### Step 2: Preparation of Benzyl (3S,6S,9aS)-6-amino-5-oxooctahydro-1H-pyrrolo[1,2-a]azepine-3-carboxylate

To a solution of benzyl (3S,6S,9aS)-6-((tert-butoxycarbonyl)amino)-5-oxooctahydro-1H-pyrrolo[1,2-a]azepine-3-carboxylate (663 mg, 1.64 mmol, 1.0 eq) in CH₂Cl₂ (16 mL) was added trifluoroacetic acid (8 mL), and the resulting mixture was stirred for 2 h at room temperature. The reaction mixture was subsequently cooled (0° C.) and saturated aqueous NaHCO₃ was carefully added to basify the mixture (adjusted to pH=8-9). The resulting mixture was extracted with CH₂Cl₂ (10 mL×3), and the combined organic layers were washed with brine (10 mL×2), dried over with anhydrous Na₂SO₄, then concentrated under reduced pressure to give crude benzyl (3S,6S,9aS)-6-amino-5-oxooctahydro-1H-pyrrolo[1,2-a]azepine-3-carboxylate (498 mg, 1.64 mmol, quant) as a white solid, which was used in next step directly without further purification. LCMS (ESI): m/z=303 [M+H]⁺.

### Step 3: Preparation of Benzyl (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylate

A solution of 5-[(diethoxyphosphoryl)difluoromethyl]-1-benzothiophene-2-carboxylic acid (597 mg, 1.64 mmol, 1 eq), EDCI (412 mg, 2.13 mmol, 1.3 eq), HOBt (287 mg, 2.13 mmol, 1.3 eq) and N,N-diisopropylethylamine (635 mg, 4.92 mmol, 3 eq) in CH₂Cl₂ (15 mL) was stirred for 30 min, followed by addition of benzyl (3S,6S,9aS)-6-amino-5-oxooctahydro-1H-pyrrolo[1,2-a]azepine-3-carboxylate (498 mg, 1.64 mmol, 1 eq). The mixture was stirred over 12 h at room temperature, followed by addition of H₂O (10 mL). The biphasic mixture was extracted with CH₂Cl₂ (10 mL×3). The organic layers were combined and washed with brine (20 mL), dried over anhydrous Na₂SO₄, and concentrated under reduced pressure. The residue was purified by flash column chromatography to afford benzyl (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylate (530 mg, 817 μmol, 50%) as a white solid. LCMS (ESI): m/z=649 [M+H]⁺.

### Step 4: Preparation of (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylic acid

A mixture of benzyl (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylate (510 mg, 786 μmol, 1 eq) and 10% wt wet Pd/C (50 mg) in MeOH (15 mL) was stirred under H₂ (g) atmosphere. After complete consumption of starting material (as judged by LCMS), the reaction mixture filtered by passing through a pad of Celite®. The filtrate was concentrated under reduced pressure, and the residue was purified by flash column chromatography to afford (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylic acid (376 mg, 673 μmol, 86% yield) as an off-white solid. LCMS (ESI): m/z=559 [M+H]⁺.

### Step 5: Preparation of Diethyl (difluoro(2-(((3S,6S,9aS)-3-(methyl(phenyl)carbamoyl)-5-oxooctahydro-1H-pyrrolo[1,2-a]azepin-6-yl)carbamoyl)benzo[b]thiophen-5-yl)methyl)phosphonate

A solution of (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylic acid (120 mg, 214 μmol, 1 eq), 2-chloro-1-methylpyridinium iodide (164 mg, 642 μmol, 3.0 eq), and N,N-diisopropylethylamine (82.9 mg, 642 μmol, 3.0 eq) in CH₂Cl₂ (15 mL) was stirred for 30 min at room temperature, followed by addition of N-methylaniline (22.9 mg, 214 μmol, 1.0 eq). After stirring for 12 h, The reaction mixture was diluted with H₂O (10 mL) and the resulting biphasic mixture was extracted with CH₂Cl₂ (10 mL×3). The combined organic layers were washed with brine (10 mL), dried over anhydrous Na₂SO₄, and concentrated under reduced pressure. The residue was purified by flash column chromatography to afford diethyl (difluoro(2-(((3S,6S,9aS)-3-(methyl(phenyl)carbamoyl)-5-oxooctahydro-1H-pyrrolo[1,2-a]azepin-6-yl)carbamoyl)benzo[b]thiophen-5-yl)methyl)phosphonate (90 mg, 138 μmol, 65% yield) as a white solid. LCMS (ESI) m/z=648 [M+H]⁺.

### Step 6: Preparation of (difluoro(2-(((3S,6S,9aS)-3-(methyl(phenyl)carbamoyl)-5-oxooctahydro-1H-pyrrolo[1,2-a]azepin-6-yl)carbamoyl)benzo[b]thiophen-5-yl)methyl)phosphonic acid (111)

To a cooled (0° C.) solution of diethyl (difluoro(2-(((3S,6S,9aS)-3-(methyl(phenyl)carbamoyl)-5-oxooctahydro-1H-pyrrolo[1,2-a]azepin-6-yl)carbamoyl)benzo[b]thiophen-5-yl)methyl)phosphonate (50 mg, 77.1 μmol, 1.0 eq) in CH₂Cl₂ (15 mL) was added bromo trimethylsilane (235 mg, 1.54 mmol, 20.0 eq) in a dropwise manner. After the addition, the reaction mixture was allowed to warm to room temperature and stirred for an additional 12 h. After completion consumption of starting material (as judged by LCMS), the reaction mixture was quenched by adding H₂O (5 mL) and the resulting biphasic mixture was extracted with CH₂Cl₂ (10 mL×3). The organic layers were combined and washed with brine (20 mL), dried over with anhydrous Na₂SO₄, then concentrated under reduced pressure. The residue was purified by reverse phase HPLC to afford (difluoro(2-(((3S,6S,9aS)-3-(methyl(phenyl)carbamoyl)-5-oxooctahydro-1H-pyrrolo[1,2-a]azepin-6-yl)carbamoyl)benzo[b]thiophen-5-yl)methyl)phosphonic acid (111) (2.1 mg, 3.5 μmol, 4.5% yield) as a white solid. LCMS (ESI) m/z=592 [M+H]⁺; ¹H NMR (400 MHz, DMSO-d₆) δ 8.74 (d, J=6.7 Hz, 1H), 8.29 (s, 1H), 8.13-7.96 (m, 2H), 7.65 (d, J=7.3 Hz, 1H), 7.53-7.25 (m, 5H), 4.67-4.55 (m, 1H), 4.41-4.27 (m, 1H), 3.99-3.85 (m, 1H), 3.15 (s, 3H), 2.16-1.62 (m, 10H).

The following compounds in Table 31 were prepared according to the representative procedure described above for the synthesis of (difluoro(2-(((3S,6S,9aS)-3-(methyl(phenyl)carbamoyl)-5-oxooctahydro-1H-pyrrolo[1,2-a]azepin-6-yl)carbamoyl)benzo[b]thiophen-5-yl)methyl)phosphonic acid and utilizing the appropriate starting materials and modifications.

TABLE 31

| Compound | Name | Structure | LCMS | NMR |
|---|---|---|---|---|
| 112 | ((2- (((3S,6S,10aS)- 3-([1,1′- biphenyl]-4- ylcarbamoyl)-5- oxodecahydro- pyrrolo[1,2- a]azocin-6- yl)carbamoyl) benzo[b] thiophen-5-yl) difluoromethyl) phosphonic acid | 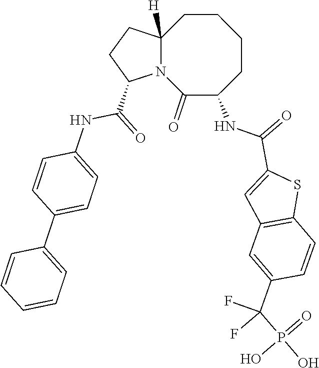 [SMILES: O=C(N[C@H]1CCCC[C@H]2CC[C@@H](C(=O)Nc3ccc(-c4ccccc4)cc3)N2C1=O)c1cc2cc(C(F)(F)P(=O)(O)O)ccc2s1] | 668.4 [M + H]⁺ | ¹H NMR (400 MHz, methanol-d₄) δ 8.15 (s, 2H), 8.00 (d, J = 8.4 Hz, 1H), 7.73 − 7.64 (m, 3H), 7.63 − 7.57 (m, 4H), 7.43 (t, J = 7.6 Hz, 2H), 7.35 − 7.29 (m, 1H), 5.09 (t, J = 8.8 Hz, 1H), 4.60 (t, J = 8.4 Hz, 1H), 4.48 (m, 1H), 2.42 − 2.27 (m, 2H), 2.24 − 1.95 (m, 6H), 1.93 − 1.81 (m, 2H), 1.80 − 1.61 (m, 2H) |
| 113 | (difluoro(2- (((3S,6S, 10aS)-3-(3- morpho- linoazetidine- 1-carbonyl)-5- oxodecahydr opyrrolo[1,2- a]azocin-6- yl)carbamoyl) benzo[b]thio phen-5- yl)methyl)ph osphonic acid | 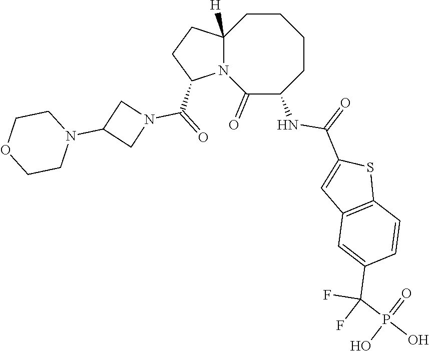 [SMILES: O=C(N[C@H]1CCCC[C@H]2CC[C@@H](C(=O)N3CC(N4CCOCC4)C3)N2C1=O)c1cc2cc(C(F)(F)P(=O)(O)O)ccc2s1] | 641.1 [M + H]⁺ | ¹H NMR (400 MHz, methanol-d₄) δ 8.20 (s, 1H), 8.13 (s, 1H), 7.91 (d, J = 8.4 Hz, 1H), 7.77 (d, J = 8.8 Hz, 1H), 5.07 − 4.97 (m, 1H), 4.54 (t, J = 8.0 Hz, 1H), 4.47 − 4.38 (m, 2H), 4.15 − 4.05 (m, 1H), 3.95 − 3.92 (m, 1H), 3.82 − 3.78 (m, 1H), 3.72 − 3.66 (m, 4H), 3.27 − 3.16 (m, 1H), 2.41 (s, 4H), 2.30 − 2.14 (m, 2H), 2.08 − 1.90 (m, 6H), 1.88 − 1.75 (m, 2H), 1.73 − 1.55 (m, 2H) |
| 114 | (difluoro(2- (((3′S,6′S, 10aR)-3′- (3-(morpholine- 4-carbonyl) azetidine- 1-carbonyl)-5′- oxooctahydro- 1′H-spiro [cyclopropane- 1,9′-pyrrolo[1,2- a]azocin]-6′- yl)carbamoyl) benzo[b]thiophen- 5-yl)methyl) phosphonic acid | 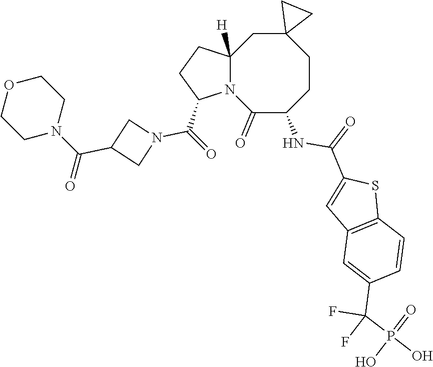 [SMILES: O=C(N[C@H]1CCC2(CC2)C[C@H]2CC[C@@H](C(=O)N3CC(C(=O)N4CCOCC4)C3)N2C1=O)c1cc2cc(C(F)(F)P(=O)(O)O)ccc2s1] | 695.3 [M + H]⁺ | ¹H NMR (400 MHz, methanol-d₄) δ 8.19 − 8.14 (m, 1H), 8.14 − 8.09 (m, 1H), 7.96 − 7.88 (m, 1H), 7.74 (d, J = 8.4 Hz, 1H), 5.19 − 5.07 (m, 1H), 4.70 − 4.63 (m, 1H), 4.50 − 4.43 (m, 2H), 4.42 − 4.36 (m, 1H), 4.33 − 4.20 (m, 1H), 4.15 − 4.04 (m, 1H), 3.86 − 3.71 (m, 1H), 3.70 − 3.54 (m, 6H), 3.40 − 3.34 (m, 1H), 2.66 − 2.46 (m, 2H), 2.31 − 2.16 (m, 2H), 2.09 − 1.66 (m, 5H), 1.24 (d, J = 11.6 Hz, 1H), 0.78 − 0.66 (m, 2H), 0.58 − 0.44 (m, 2H), 0.36 (d, J = 9.2 Hz, 1H) |
| 115 | (difluoro(2- (((3′S,6′S, 10a′S)-3′-(3- (morpholine-4- carbonyl) azetidine-1- carbonyl)-5′- oxooctahydro- 5′H-spiro [cyclopropane- 1,8′-pyrrolo[1,2- a]azocin]-6′- yl)carbamoyl) benzo[b]thiophen- 5-yl)methyl) phosphonic acid | 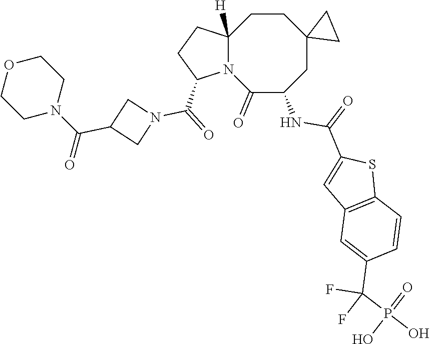 [SMILES: O=C(N[C@H]1CC2(CC[C@H]3CC[C@@H](C(=O)N4CC(C(=O)N5CCOCC5)C4)N3C1=O)CC2)c1cc2cc(C(F)(F)P(=O)(O)O)ccc2s1] | 695.3 [M + H]⁺ | ¹H NMR (400 MHz, methanol-d₄) δ 8.20 − 8.12 (m, 1H), 8.10 − 8.03 (m, 1H), 7.94 − 7.85 (m, 1H), 7.74 (d, J = 8.4 Hz, 1H), 5.04 − 4.94 (m, 1H), 4.78 − 4.69 (m, 1H), 4.51 − 4.42 (m, 2H), 4.42 − 4.35 (m, 1H), 4.34 − 4.20 (m, 1H), 4.13 − 4.05 (m, 1H), 3.84 − 3.71 (m, 1H), 3.68 − 3.48 (m, 6H), 3.36 (m, 1H), 2.28 − 2.15 (m, 2H), 2.11 − 1.96 (m, 1H), 1.95 − 1.80 (m, 4H), 1.78 − 1.54 (m, 3H), 1.36 − 1.27 (m, 1H), 0.62 − 0.42 (m, 4H) |
| 116 | ((2-(((3S, 6S,10aS)-3- (((3S,4S)- 1-acetyl-4- hydroxypyrrolidin- 3-yl)carbamoyl)-5- oxodecahydro- pyrrolo [1,2-a]azocin-6- yl)carbamoyl) benzo[b] thiophen-5- yl)difluoromethyl) phosphonic acid | 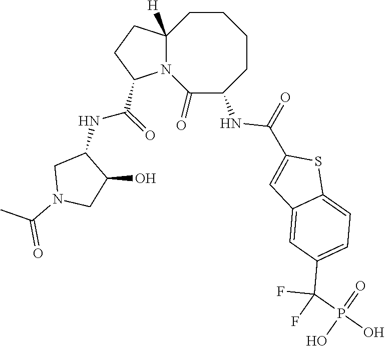 [SMILES: CC(=O)N1C[C@H](NC(=O)[C@@H]2CC[C@@H]3CCCC[C@H](NC(=O)c4cc5cc(C(F)(F)P(=O)(O)O)ccc5s4)C(=O)N32)[C@@H](O)C1] | 643.1 [M + H]⁺ | ¹H NMR (400 MHz, methanol-d₄) δ 8.19 (s, 1H), 8.12 (s, 1H), 7.91 (d, J = 8.4 Hz, 1H), 7.76 (d, J = 8.4 Hz, 1H), 5.06 − 4.98 (m, 1H), 4.45 − 4.33 (m, 2H), 4.20 (d, J = 2.4 Hz, 1H), 4.16 − 4.11 (m, 1H), 3.90 − 3.86 (m, 1H), 3.78 − 3.70 (m, 1H), 3.60 − 3.56 (m, 1H), 3.52 − 3.39 (m, 2H), 2.27 − 2.15 (m, 2H), 2.06 (d, J = 4.0 Hz, 3H), 2.02 − 1.94 (m, 5H), 1.87 − 1.76 (m, 2H), 1.73 − 1.58 (m, 2H) |
| 117 | (difluoro(2- (((3S,6S,10aS)- 5-oxo-3- (3-(pyridin-3- yl)azetidine-1- carbonyl) decahydropyrrolo [1,2-a]azocin-6- yl)carbamoyl) benzo[b]thiophen- 5-yl)methyl) phosphonic acid | 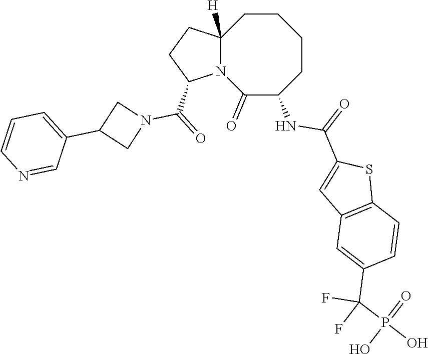 [SMILES: O=C(N[C@H]1CCCC[C@H]2CC[C@@H](C(=O)N3CC(c4cccnc4)C3)N2C1=O)c1cc2cc(C(F)(F)P(=O)(O)O)ccc2s1] | 633.1 [M + H]⁺ | ¹H NMR (400 MHz, methanol-d₄) δ 8.59 (d, J = 17.2 Hz, 1H), 8.47 (s, 1H), 8.26 − 8.09 (m, 3H), 7.97 − 7.85 (m, 1H), 7.80 − 7.67 (m, 1H), 7.61 − 7.38 (m, 1H), 5.11 − 4.96 (m, 1H), 4.73 − 4.59 (m, 2H), 4.57 − 4.38 (m, 3H), 3.82 (s, 2H), 2.33 − 2.18 (m, 2H), 2.15 − 1.93 (m, 6H), 1.92 − 1.56 (m, 4H) |
| 118A | (difluoro(2- (((3S,6S,10a S)-5-oxo-3-(3- (tetrahydro- 2H-pyran-4- yl)azetidine-1- carbonyl) decahydropyrrolo- [1,2a]azocin-6- yl)carbamoyl) benzo[b]thiophen- 5-yl)methyl) phosphonic acid | 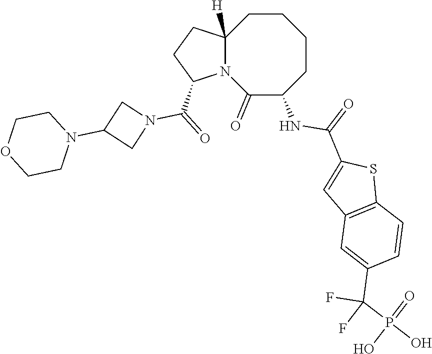 [SMILES: O=C(N[C@H]1CCCC[C@H]2CC[C@@H](C(=O)N3CC(N4CCOCC4)C3)N2C1=O)c1cc2cc(C(F)(F)P(=O)(O)O)ccc2s1] | 640.1 [M + H]⁺ | ¹H NMR (400 MHz, methanol-d₄) δ 8.23 − 8.12 (m, 2H), 7.95 (d, J = 8.4 Hz, 1H), 7.84 − 7.67 (m, 1H), 5.13 − 4.99 (m, 1H), 4.64 − 4.39 (m, 3H), 4.28 (d, J = 7.2 Hz, 1H), 4.01 − 3.87 (m, 2H), 3.85 3.62 (m, 2H), 3.52 − 3.35 (m, 1H), 2.53 − 2.16 (m, 3H), 2.11 − 1.94 (m, 6H), 1.91 − 1.75 (m, 4H), 1.73 − 1.57 (m, 4H), 1.41 − 1.07 (m, 2H) |
| 118B | ((2-(((3S,6S,10a S)-3-(4-(1H- pyrazole-4- carbonyl) piperazine-1- carbonyl)-5- oxodecahydro- pyrrolo [1,2-a]azocin-6- yl)carbamoyl) benzo[b] thiophen-5-yl) difluoromethyl) phosphonic acid | 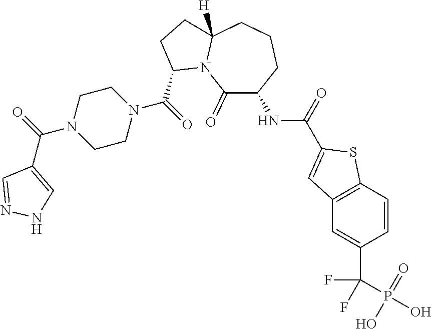 [SMILES: O=C(N[C@H]1CCC[C@H]2CC[C@@H](C(=O)N3CCN(C(=O)c4cn[nH]c4)CC3)N2C1=O)c1cc2cc(C(F)(F)P(=O)(O)O)ccc2s1] | 679.4 [M + H]⁺ | ¹H NMR (400 MHz, methanol-d₄) δ 8.18 (s, 1H), 8.13 (s, 1H), 7.94 − 7.89 (m, 3H), 7.74 (d, J = 8.8 Hz, 1H), 5.08 − 5.01 (m, 1H), 4.50 − 4.39 (m, 1H), 3.93 − 3.74 (m, 6H), 3.73 − 3.51 (m, 3H), 2.37 − 2.22 (m, 2H), 2.21 − 2.09 (m, 1H), 2.07 − 1.93 (m, 5H), 1.89 − 1.75 (m, 2H), 1.73 − 1.59 (m, 2H) |
| 119 | (difluoro(2- (((3S,6S,9aS)- 5-oxo-3-(3- (tetrahydro- 2H-pyran-4- yl)azetidine- 1-carbonyl) octahydro-1H- pyrrolo[1,2- a]azepin-6- yl)carbamoyl) benzo[b] thiophen-5- yl)methyl) phosphonic acid | 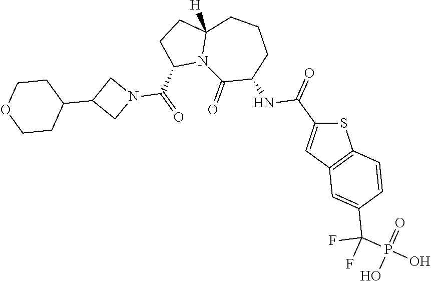 [SMILES: O=C(N[C@H]1CCC[C@H]2CC[C@@H](C(=O)N3CC(C4CCOCC4)C3)N2C1=O)c1cc2cc(C(F)(F)P(=O)(O)O)ccc2s1] | 626.5 [M + H]⁺ | ¹H NMR (400 MHz, methanol-d₄) δ 8.20 (s, 1H), 8.12 (d, J = 1.6 Hz, 1H), 7.94 (d, J = 8.4 Hz, 1H), 7.76 (d, J = 8.4 Hz, 1H) , 4.74 (d, J = 10.8 Hz, 1H), 4.58 − 4.50 (m, 1H), 4.32 − 4.18 (m, 1H), 4.15 − 4.00 (m, 2H), 3.99 − 3.87 (m, 3H), 3.80 − 3.66 (m, 1H), 3.47 − 3.37 (m, 2H) , 2.50 − 2.37 (m, 1H), 2.34 − 2.23 (m, 1H), 2.16 − 2.02 (m, 3H), 1.97 − 1.73 (m, 7H), 1.68 − 1.55 (m, 2H), 1.24 − 1.13 (m, 2H) |
| 120 | ((2-(((3S,6S,9aS)- 3-(((3S,4S)- 1-acetyl-4- hydroxypyrrolidin- 3-yl)carbamoyl)-5- oxooctahydro-1H- pyrrolo[1,2- a]azepin-6- yl)carbamoyl) benzo[b]thiophen- 5-yl) difluoromethyl) phosphonic acid | 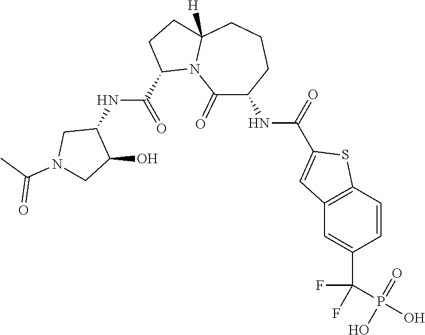 [SMILES: CC(=O)N1C[C@H](NC(=O)[C@@H]2CC[C@@H]3CCC[C@H](NC(=O)c4cc5cc(C(F)(F)P(=O)(O)O)ccc5s4)C(=O)N32)[C@@H](O)C1] | 629.2 [M + H]⁺ | ¹H NMR (400 MHz, methanol-d₄) δ 8.20 (s, 1H), 8.10 (d, J = 4.0 Hz, 1H), 7.92 (d, J = 8.8 Hz, 1H), 7.77 (d, J = 8.8 Hz, 1H), 4.72 (d, J = 10.8 Hz, 1H), 4.57 − 4.44 (m, 1H), 4.24 − 4.10 (m, 2H), 4.09 − 4.00 (m, 1H), 3.93 − 3.75 (m, 1H), 3.74 − 3.55 (m, 1H), 3.52 − 3.38 (m, 2H), 2.33 − 2.24 (m, 1H), 2.09 (d, J = 2.8 Hz, 1H), 2.06 (d, J = 1.6 Hz, 3H), 2.06 − 1.93 (m, 3H), 1.93 − 1.73 (m, 5H) |
| 121 | ((2- (((3S,6S,9aS)- 3-([1,1′- biphenyl]-4- ylcarbamoyl)-5- oxooctahydro-1H- pyrrolo[1,2- a]azepin-6- yl)carbamoyl) benzo[b] thiophen-5- yl)difluoromethyl) phosphonic acid | 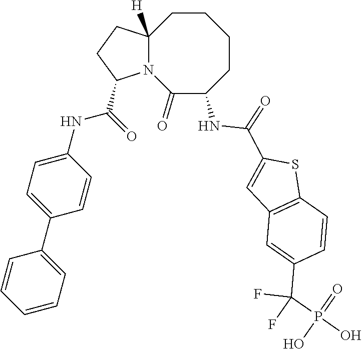 [SMILES: O=C(N[C@H]1CCCC[C@H]2CC[C@@H](C(=O)Nc3ccc(-c4ccccc4)cc3)N2C1=O)c1cc2cc(C(F)(F)P(=O)(O)O)ccc2s1] | 654.3 [M + H]⁺ | ¹H NMR (400 MHz, DMSO-d₆) δ 10.24 (s, 1H), 8.79 (d, J = 7.5 Hz, 1H), 8.28 (s, 1H), 8.16 − 7.98 (m, 2H), 7.75 − 7.55 (m, 7H), 7.49 − 7.39 (m, 2H), 7.37 − 7.24 (m, 1H), 4.71 − 4.54 (m, 2H), 4.08 − 4.01 (m, 1H), 2.30 − 2.07 (m, 2H), 2.05 − 1.71 (m, 8H) |

### Azetidine Building Block Syntheses

### Synthesis of 1-phenyl-2-(2,6-diazaspiro[3.3]heptan-2-yl)ethan-1-one

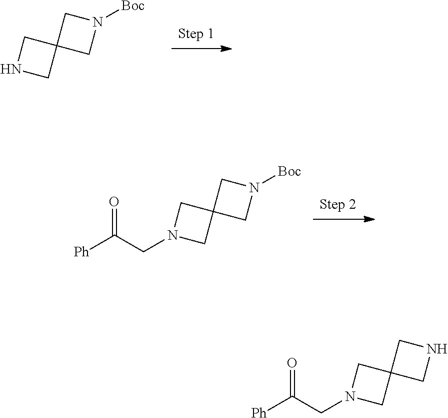 [SMILES: CC(C)(C)OC(=O)N1CC2(CNC2)C1 ; CC(C)(C)OC(=O)N1CC2(CN(CC(=O)c3ccccc3)C2)C1 ; O=C(CN1CC2(CNC2)C1)c1ccccc1]

### Step 1: Preparation of Tert-Butyl 6-(2-oxo-2-phenylethyl)-2,6-diazaspiro[3.3]heptane-2-carboxylate

To a solution of 2-phenylacetic acid (500 mg, 3.67 mmol, 1 eq) in DMF (4 mL) was added N,N-diisopropylethylamine (1.42 g, 11.0 mmol, 3 eq) and HATU (2.09 g, 5.50 mmol, 1.5 eq). After stirring for 10 min, a solution of tert-butyl 2,6-diazaspiro[3.3]heptane-2-carboxylate (945 mg, 4.77 mmol, 1.3 eq) in DMF (4 mL) and N,N-diisopropylethylamine (1.42 g, 11.0 mmol, 3 eq) was added. The mixture was stirred for an additional 1 h and subsequently diluted with water (20 mL). The mixture was extracted with EtOAc (10 mL×2). The combined organic layers were washed with saturated aqueous brine (10 mL×2), dried over sodium sulfate, filtered and concentrated under reduced pressure to give a residue. The residue was purified by flash silica gel chromatography (eluent of 0%-5%=CH₂Cl₂: MeOH) to give tert-butyl 6-(2-phenylacetyl)-2,6-diazaspiro[3.3]heptane-2-carboxylate (1.00 g, 3.16 mmol, 86.2% yield) was obtained as a white solid. LCMS (ESI) m/z=317.0 [M+H]⁺; ¹H NMR (400 MHz, CDCl₃) δ 7.36-7.29 (m, 2H), 7.28-7.22 (m, 3H), 4.18 (s, 2H), 4.11 (s, 2H), 3.47 (s, 2H), 2.81 (s, 4H), 1.72 (s, 4H), 1.43 (s, 9H).

### Step 2: Preparation of 1-phenyl-2-(2,6-diazaspiro[3.3]heptan-2-yl)ethan-1-one

To a solution of tert-butyl 6-(2-phenylacetyl)-2,6-diazaspiro[3.3]heptane-2-carboxylate (1.00 g, 3.16 mmol) in CH₂Cl₂ (6 mL) was added trifluoroacetic acid (2 mL, 0.0175 mmol, 0.006 eq) and the reaction mixture as stirred for 1 h. The resulting yellow reaction mixture was concentrated under reduced pressure to give a residue. The product 1-phenyl-2-(2,6-diazaspiro[3.3]heptan-2-yl)ethan-1-one (˜1 g, TFA salt) was obtained as a yellow oil and was used without further purification.

### Synthesis of 1-((2R,3S)-2-methylazetidin-3-yl)-1H-imidazole

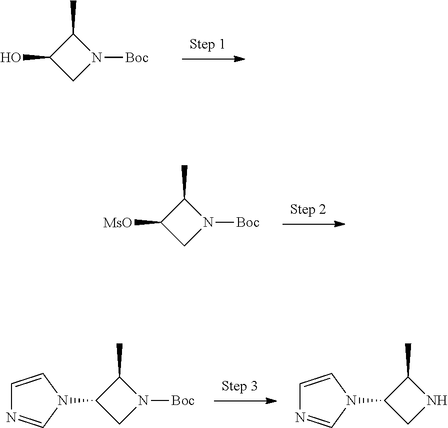

### Step 1: Preparation of Tert-Butyl (2R,3R)-2-methyl-3-((methylsulfonyl)oxy)azetidine-1-carboxylate

To a solution of tert-butyl (2R,3R)-3-hydroxy-2-methylazetidine-1-carboxylate (500 mg, 2.67 mmol, 1.0 eq) and Et₃N (810 mg, 8.01 mmol, 3.0 eq) in CH₂Cl₂ (20 mL) was added methanesulfonyl chloride (335 mg, 2.93 mmol, 1.1 eq). The reaction mixture was stirred at room temperature for 14 h, followed by addition of H₂O (10 mL). The resulting biphasic mixture was extracted with CH₂Cl₂ (20 mL×3). The organic layers were combined, washed with brine (20 mL), dried over anhydrous Na₂SO₄, and concentrated under reduced pressure. The residue was purified by Biotage® C18 column to afford tert-butyl (2R,3R)-2-methyl-3-((methylsulfonyl)oxy)azetidine-1-carboxylate (710 mg, 2.67 mmol, 100% yield) as a colorless oil. LCMS (ESI) m/z=210 [(M−56)+H]⁺.

### Step 2: Preparation of Tert-Butyl (2R,3S)-3-(1H-imidazol-1-yl)-2-methylazetidine-1-carboxylate

To a cooled (0° C.) solution of 1H-imidazole (599 mg, 8.81 mmol, 3.3 eq) in DMF (10 mL) under a constant stream of N₂ (g), was added NaH (352 mg, 8.81 mmol, 3.3 eq) in portions. The mixture was stirred until gas evolution ceased (˜30 min). To the mixture as added a solution of tert-butyl (2R,3R)-2-methyl-3-((methylsulfonyl)oxy)azetidine-1-carboxylate (710 mg, 2.67 mmol, 1.0 eq) in DMF (3 mL). The reaction mixture was subsequently heated to 80° C. After stirring for 72 h, the reaction mixture was cooled to 0° C. and saturated aqueous NH₄Cl (10 mL) was added. The mixture was extracted with EtOAc (20 mL×3). The organic layers were combined, washed with brine (10 mL), dried over anhydrous Na₂SO₄, and concentrated under reduced pressure. The residue was purified by Biotage® C18 column to afford tert-butyl (2R,3S)-3-(1H-imidazol-1-yl)-2-methylazetidine-1-carboxylate (73.0 mg, 0.31 mmol, 12% yield) as a colorless oil. LCMS (ESI) m/z=238 [M+H]⁺.

### Step 3: Preparation of 1-((2R,3S)-2-methylazetidin-3-yl)-1H-imidazole

To a solution of tert-butyl (2R,3S)-3-(1H-imidazol-1-yl)-2-methylazetidine-1-carboxylate (73.0 mg, 0.31 mmol, 1.0 eq) in CH₂Cl₂ (3 mL) was added TFA (1 mL), and the resulting mixture was stirred at room temperature. After stirring for 16 h, the reaction mixture was concentrated under reduced pressure to give 1-((2R,3S)-2-methylazetidin-3-yl)-1H-imidazole (40 mg, TFA salt) as a white solid, which was used without further purification. LCMS (ESI) m/z=138 [M+H]⁺.

### ((2-(((3S,6S,10aS)-3-(3-butyramido-3-(pyridin-2-yl)azetidine-1-carbonyl)-5-oxodecahydropyrrolo[1,2-a]azocin-6-yl)carbamoyl)benzo[b]thiophen-5-yl)difluoromethyl)phosphonic acid (122)

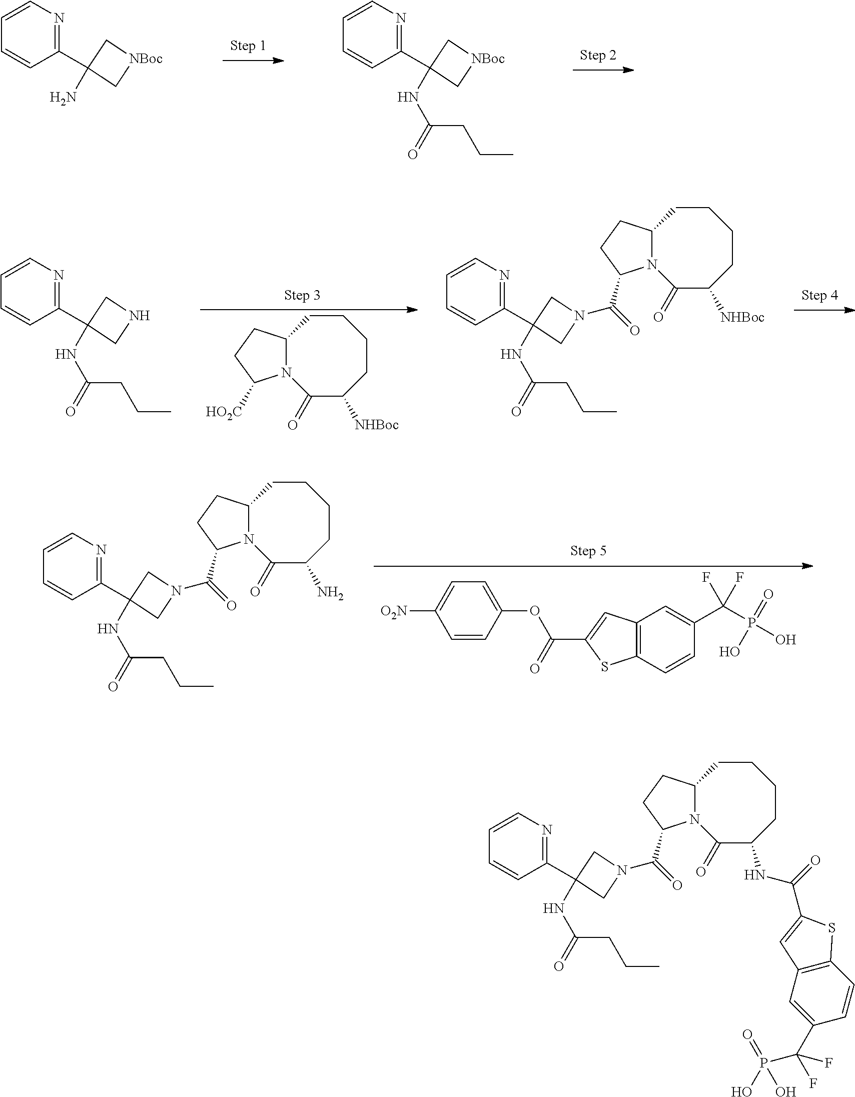

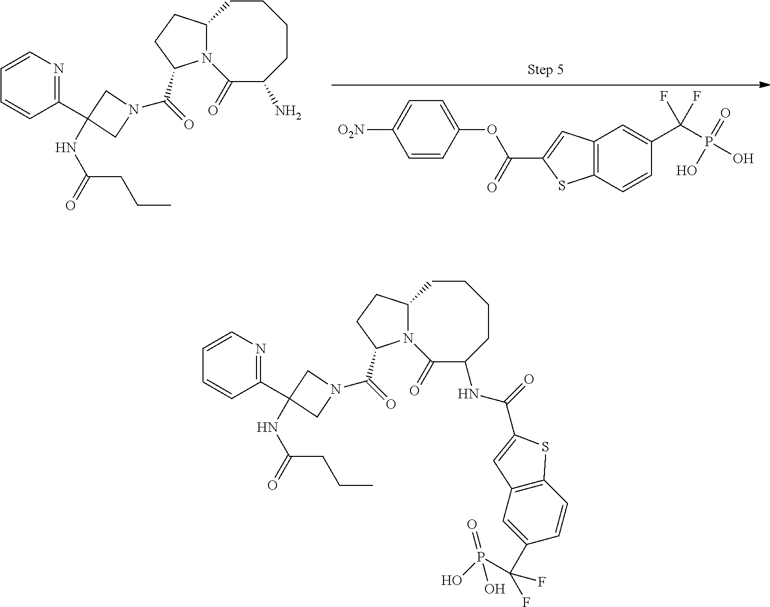

### Step 1: Preparation of Tert-Butyl 3-butyramido-3-(pyridin-2-yl)azetidine-1-carboxylate

To a solution of butyric acid (95 mg, 1.1 mmol, 0.9 eq) in DMF (3 mL) was added HATU (0.69 g, 1.8 mmol, 1.5 eq) and N,N-diisopropylethylamine (0.46 g, 3.6 mmol, 3 eq). The mixture was stirred for 10 minutes, followed by addition of tert-butyl 3-amino-3-(pyridin-2-yl)azetidine-1-carboxylate (0.30 g, 1.2 mmol, 1 eq). After stirring for an additional 1 h, the reaction mixture turned brown. The reaction mixture was diluted with water (30 mL) and extracted with EtOAc (30 mL×3). The combined organic layers were washed with saturated aqueous brine (30 mL×3), dried over Na₂SO₄, filtered and concentrated under reduced pressure to give a residue. The residue was purified by flash silica gel chromatography (SepaFlash Column, 0%-100% EtOAc/petroleum ether gradient) to give tert-butyl 3-butyramido-3-(pyridin-2-yl)azetidine-1-carboxylate (0.26 g, 68% yield) as white solids. LCMS (ESI) m/z=320.1 [M+H]⁺.

### Step 2: Preparation of N-(3-(pyridin-2-yl)azetidin-3-yl)butyramide

A solution of tert-butyl 3-butyramido-3-(pyridin-2-yl)azetidine-1-carboxylate (0.24 g, 0.75 mmol, 1 eq) in CH₂Cl₂ (2 mL) and TFA (1 mL) was stirred at room temperature. After 1 h, the resulting yellow solution concentrated under reduced pressure to give N-(3-(pyridin-2-yl)azetidin-3-yl)butyramide (0.24 g, TFA salt) which was used into the next step without further purification.

The following intermediates in Table 32 were prepared according to the representative procedures (Step1 and Step 2) described above starting from tert-butyl 3-butyramido-3-(pyridin-2-yl)azetidine-1-carboxylate and utilizing appropriate starting materials and modifications.

TABLE 32

| Name | Structure | LCMS |
|---|---|---|
| N-(3-(pyridin-2-yl)azetidin-3- yl)cyclopropanecarboxamide | 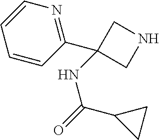 [SMILES: O=C(NC1(c2ccccn2)CNC1)C1CC1] | 218.1 [M + H]⁺ |
| 4,4,4-trifluoro-N-(3-(pyridin-2- yl)azetidin-3-yl)butanamide | 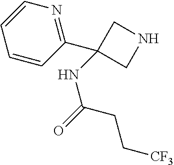 [SMILES: O=C(CCC(F)(F)F)NC1(c2ccccn2)CNC1] | 218.1 [M + H]⁺ |

### Step 3: Preparation of Tert-Butyl ((3S,6S,10aS)-3-(3-butyramido-3-(pyridin-2-yl)azetidine-1-carbonyl)-5-oxodecahydropyrrolo[1,2-a]azocin-6-yl)carbamate

To a solution of (3S,6S,10aS)-6-((tert-butoxycarbonyl)amino)-5-oxodecahydropyrrolo[1,2-a]azocine-3-carboxylic acid (0.36 g, 1.1 mmol, 1 eq) in DMF (3 mL) was added N,N-diisopropylethylamine (0.43 g, 3.3 mmol, 3 eq) and HATU (0.63 g, 1.6 mmol, 1.5 eq). After 10 min, N-(3-(pyridin-2-yl)azetidin-3-yl)butyramide (0.24 g, 1.1 mmol, 1 eq) was added. After 1 h, the brown solution was diluted with water (20 mL) and extracted with EtOAc (20 mL×3). The combined organic layers were washed with saturated aqueous brine 60 mL (20 mL×3), dried over Na₂SO₄, filtered and concentrated under reduced pressure to give a residue. The residue was purified by flash silica gel chromatography (SepaFlash Silica Flash Column, 0%-100% EtOAc/petroleum ether) to give tert-butyl ((3S,6S,10aS)-3-(3-butyramido-3-(pyridin-2-yl)azetidine-1-carbonyl)-5-oxodecahydropyrrolo[1,2-a]azocin-6-yl)carbamate (0.29 g, 51% yield) as a white solid. LCMS (ESI) m/z=528.2 [M+H]⁺; ¹H NMR (400 MHz, DMSO-d₆) δ 9.11-8.95 (m, 1H), 8.61 (d, J=4.4 Hz, 1H), 7.82-7.73 (m, 1H), 7.43-7.26 (m, 2H), 6.76-6.67 (m, 1H), 4.66-4.53 (m, 1H), 4.32 (td, J=8.8, 9.2 Hz, 2H), 4.19-4.08 (m, 2H), 4.07-3.99 (m, 1H), 2.25-2.13 (m, 3H), 1.99 (s, 1H), 1.87-1.65 (m, 6H), 1.54 (dt, J=7.8, 11.2 Hz, 6H), 1.38-1.30 (m, 9H), 1.17 (t, J=7.2 Hz, 1H), 0.88 (td, J=5.2, 5.2 Hz, 3H)

### Step 4: Preparation of N-(1-((3S,6S,10aS)-6-amino-5-oxodecahydropyrrolo[1,2-a]azocine-3-carbonyl)-3-(pyridin-2-yl)azetidin-3-yl)butyramide

A solution of tert-butyl ((3S,6S,10aS)-3-(3-butyramido-3-(pyridin-2-yl)azetidine-1-carbonyl)-5-oxodecahydropyrrolo[1,2-a]azocin-6-yl)carbamate (0.22 g, 0.42 mmol, 1 eq) in CH₂Cl₂ (1 mL) and TFA (0.5 mL) was stirred at room temperature. After 1 h, the resulting yellow solution was concentrated under reduced pressure to give N-(1-((3S,6S,10aS)-6-amino-5-oxodecahydropyrrolo[1,2-a]azocine-3-carbonyl)-3-(pyridin-2-yl)azetidin-3-yl)butyramide (0.22 g, TFA salt) which was used into the next step without further purification.

### Step 5: Preparation of ((2-(((3S,6S,10aS)-3-(3-butyramido-3-(pyridin-2-yl)azetidine-1-carbonyl)-5-oxodecahydropyrrolo[1,2-a]azocin-6-yl)carbamoyl)benzo[b]thiophen-5-yl)difluoromethyl)phosphonic acid (122)

To a solution of N-(1-((3S,6S,10aS)-6-amino-5-oxodecahydropyrrolo[1,2-a]azocine-3-carbonyl)-3-(pyridin-2-yl)azetidin-3-yl)butyramide (80 mg, 0.19 mmol, 1 eq) in DMF (1 mL) was added (difluoro(2-((4-nitrophenoxy)carbonyl)benzo[b]thiophen-5-yl)methyl)phosphonic acid (80 mg, 0.19 mmol, 1 eq), N,N-diisopropylethylamine (73 mg, 0.57 mmol, 3 eq) and HOBt (25 mg, 0.19 mmol, 1 eq). The mixture was stirred at 25° C. for 10 min and the yellow mixture was subsequently filtered. The filtrate was purified by prep-HPLC (Phenomenex Luna C18 150×25 mm×10 um; water (0.1% TFA)-ACN; B %: 32%-62% over 10 min) to yield ((2-(((3S,6S,10aS)-3-(3-butyramido-3-(pyridin-2-yl)azetidine-1-carbonyl)-5-oxodecahydropyrrolo[1,2-a]azocin-6-yl)carbamoyl)benzo[b]thiophen-5-yl)difluoromethyl)phosphonic acid (122) (40 mg, 5.6% yield) as a white solid. LCMS (ESI) m/z=718.4 [M+H]⁺; ¹H NMR (400 MHz, methanol-d₄) δ 8.67-8.33 (m, 1H), 8.21-8.12 (m, 1H), 8.03 (s, 1H), 8.02-7.93 (m, 1H), 7.88 (d, J=8.4 Hz, 1H), 7.73 (d, J=8.0 Hz, 1H), 7.67 (dd, J=8.4, 6.4 Hz, 1H), 7.58-7.51 (m, 1H), 5.06-4.93 (m, 2H), 4.84-4.77 (m, 1H), 4.68-4.60 (m, 1H), 4.55-4.47 (m, 1H), 4.55-4.47 (m, 1H), 4.45 (d, J=10.0 Hz, 1H), 4.36-4.26 (m, 1H), 2.34-2.21 (m, 4H), 2.14-1.92 (m, 6H), 1.91-1.79 (m, 2H), 1.75-1.68 (m, 1H), 1.68-1.51 (m, 3H), 0.90 (td, J=7.2, 12.0 Hz, 3H).

The following compounds in Table 33 were prepared according to the representative procedures described above starting from (3S,6S,10aS)-6-((tert-butoxycarbonyl)amino)-5-oxodecahydropyrrolo[1,2-a]azocine-3-carboxylic acid and utilizing appropriate starting materials and modifications.

TABLE 33

| Compound | Name | Structure | LCMS | NMR |
|---|---|---|---|---|
| 123 | ((2-(((3S,6S, 10aS)-3-(3- (cyclopropane- carboxamido)- 3-(pyridin-2-yl) azetidine- 1-carbonyl)-5- oxodeca- hydropyrrolo [1,2-a]azocin- 6-yl)carbamoyl) benzo[b] thiophen-5-yl) difluoromethyl) phosphonic acid | 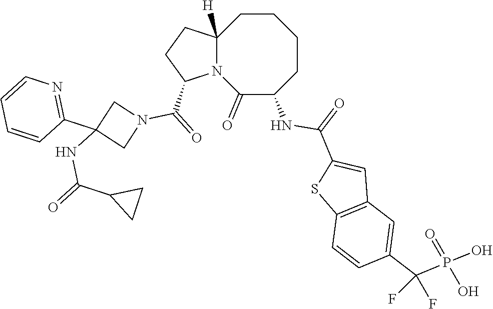 [SMILES: O=C(N[C@H]1CCCC[C@H]2CC[C@@H](C(=O)N3CC(NC(=O)C4CC4)(c4ccccn4)C3)N2C1=O)c1cc2cc(C(F)(F)P(=O)(O)O)ccc2s1] | 716.4 [M + H]⁺ | ¹H NMR (400 MHZ, methanol-d₄) δ 8.65- 8.47 (m, 1H), 8.19-8.08 (m, 2H), 7.98-7.87 (m, 1H), 7.85-7.61 (m, 2H), 7.41 (d, J = 8.0 Hz, 1H), 7.32-7.29 (m, 1H), 5.05-4.99 (m, 1H), 4.79-4.66 (m, 1H), 4.61-4.48 (m, 1H), 4.47-4.37 (m, 2H), 4.28 (d, J = 10.4 Hz, 1H), 2.38-2.20 (m, 2H), 2.14-1.93 (m, 6H), 1.90-1.77 (m, 2H), 1.74-1.71 (m, 1H), 1.69-1.60 (m, 1H), 1.37-1.33 (m, 2H), 0.90-0.71 (m, 4H) |
| 124 | (difluoro(2- (((3S,6S, 10aS)- 5-oxo-3-(3- (pyridin-2-yl)- 3-(4,4,4- trifluoro- butanamido) azetidine-1- carbonyl) decahydro- pyrrolo [1,2-a]azocin- 6-yl) carbamoyl) benzo[b] thiophen-5-yl) methyl) phosphonic acid | 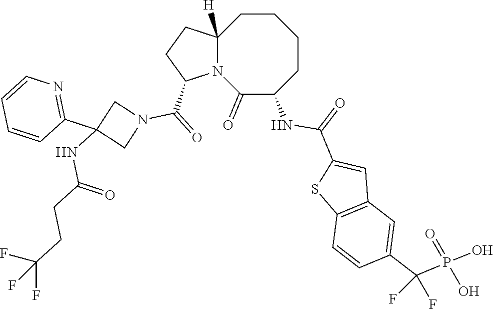 [SMILES: O=C(CCC(F)(F)F)NC1(c2ccccn2)CN(C(=O)[C@@H]2CC[C@@H]3CCCC[C@H](NC(=O)c4cc5cc(C(F)(F)P(=O)(O)O)ccc5s4)C(=O)N32)C1] | 772.4 [M + H]⁺ | ¹H NMR (400 MHZ, methanol-d₄) δ 8.64- 8.45 (m, 1H), 8.21- 8.06 (m, 2H), 7.98- 7.88 (m, 1H), 7.86- 7.76 (m, 1H), 7.75- 7.67 (m, 1H), 7.44 (d, J = 8.0 Hz, 1H), 7.36-7.27 (m, 1H), 5.07-4.98 (m, 2H), 4.78-4.71 (m, 1H), 4.62-4.50 (m, 1H), 4.48-4.40 (m, 2H), 4.29 (d, J = 10.4 Hz, 1H), 2.64-2.57 (m, 2H), 2.54-2.42 (m, 2H), 2.33-2.19 (m, 2H), 2.10-1.95 (m, 6H), 1.90-1.77 (m, 2H), 1.75-1.54 (m, 2H). |

### Pyrrolidine Building Block Synthesis

### Synthesis of rel-(trans)-4-cyclohexylpyrrolidone-3-carbonitrile

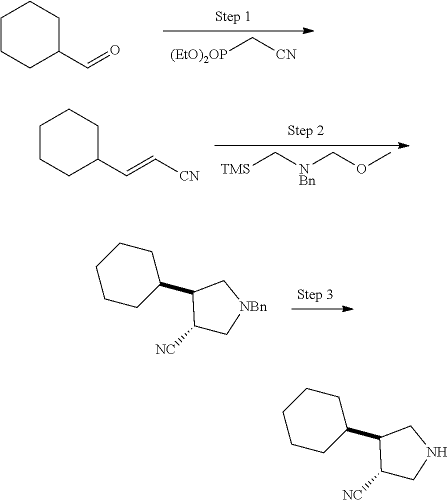

### Step 1: Preparation of (E)-3-cyclohexylacrylonitrile

To a solution of cyclohexanecarbaldehyde (500 mg, 4.45 mmol, 1.0 eq) in THF (15 mL) were added t-BuOK (998 mg, 8.90 mmol, 2.0 eq) and diethyl (cyanomethyl)phosphonate (788 mg, 4.45 mmol, 1.0 eq) at room temperature. The solution was stirred at room temperature for 1 h. After completion, the reaction mixture was quenched by adding H₂O (10 mL), then extracted with EtOAc (10 mL×3). The organic layers were combined and washed with brine (10 mL), dried over anhydrous Na₂SO₄, filtered and concentrated under reduced pressure. The residue was purified by flash column chromatography on silica gel to afford (E)-3-cyclohexylacrylonitrile (300 mg, 2.21 mmol, 50% yield) as a colorless oil. LC-MS (ESI) m/z=136 [M+H]⁺.

### Step 2: Preparation of rel-(trans)-1-benzyl-4-cyclohexylpyrrolidone-3-carbonitrile

To a solution of (E)-3-cyclohexylacrylonitrile (300 mg, 2.21 mmol, 1.0 eq) in CH₂Cl₂ (5 mL) were added N-benzyl-1-methoxy-N-((trimethylsilyl)methyl)methanamine (524 mg, 2.21 mmol, 1.0 eq) and TFA (25.1 mg, 221 μmol, 0.1 eq). The reaction mixture was stirred at room temperature for 12 h. After completion, the reaction mixture was dilute with CH₂Cl₂ (10 mL) and washed with aqueous saturated NaHCO₃(5 mL), the organic layer was separated and concentrated under reduced pressure to afford crude rel-(trans)-1-benzyl-4-cyclohexylpyrrolidine-3-carbonitrile (700 mg) as a colorless oil. LC-MS (ESI) m/z=269 [M+H]⁺.

### Step 3: Preparation of rel-(trans)-4-cyclohexylpyrrolidine-3-carbonitrile

To a solution of (trans)-1-benzyl-4-cyclohexylpyrrolidine-3-carbonitrile (700 mg) in dry 1,2-dichloroethane (15 mL) was added 1-chloroethyl carbonochloridate (3.71 g, 26.0 mmol, 10.0 eq). The resulting mixture was stirred at 70° C. for 12 h, then concentrated under reduced pressure, the crude product was dissolved in MeOH (5 mL) and stirred at 70° C. for 1 hr. After completion, the reaction solution was cooled to room temperature and concentrated under reduced pressure. The residue was purified by flash column chromatography on silica gel to afford rel-(trans)-4-cyclohexylpyrrolidine-3-carbonitrile (211 mg, 1.18 mmol, 46% yield) as a colorless oil. LC-MS (ESI) m/z=179 [M+H]⁺.

The following intermediates in Table 34 were prepared according to the representative procedures (Step1 through Step 3) described for rel-(trans)-4-cyclohexylpyrrolidine-3-carbonitrile utilizing appropriate starting materials and modifications. Compounds were prepared as racemates with trans stereochemical configuration relative to the C₃ and C₄ stereocenters within the pyrrolidine ring.

TABLE 34

| Name | Structure | LCMS |
|---|---|---|
| rel-(trans)-4-(2-oxo-1,2- dihydropyridin-4- yl)pyrrolidine-3-carbonitrile | 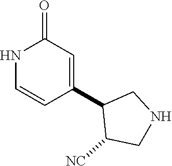 | 190 [M + H]⁺ |
| rel-(trans)-4-((tetrahydro- 2H-pyran-4-yl)methyl) pyrrolidine-3-carbonitrile | 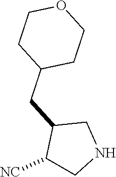 [SMILES: N#C[C@H]1CNC[C@@H]1CC1CCOCC1] | 195 [M + H]⁺ |
| rel-(trans)-4- cyclopropylpyrrolidine-3- carbonitrile | 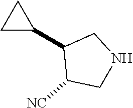 | 137 [M + H]⁺ |
| rel-(trans)-4-(6- methoxypyridin-2-yl) pyrrolidine-3-carbonitrile | 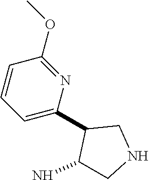 | 204 [M + H]⁺ |
| rel-(trans)-4-(1H-indazol- 6-yl)pyrrolidine-3- carbonitrile | 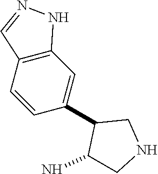 | 213 [M + H]⁺ |

tert-Butyl rel-(trans)-3-cyano-4-phenylpyrrolidine-1-carboxylate and tert-butyl (trans)-3-cyano-4-(2-oxo-1,2-dihydropyridin-4-yl)pyrrolidine-1-carboxylate were prepared according to the method describe above for the synthesis of rel-(trans)-4-cyclohexylpyrrolidine-3-carbonitrile. The racemic mixture of trans-isomers were purified under SFC conditions and the absolute stereochemistry was arbitrarily assigned as drawn.

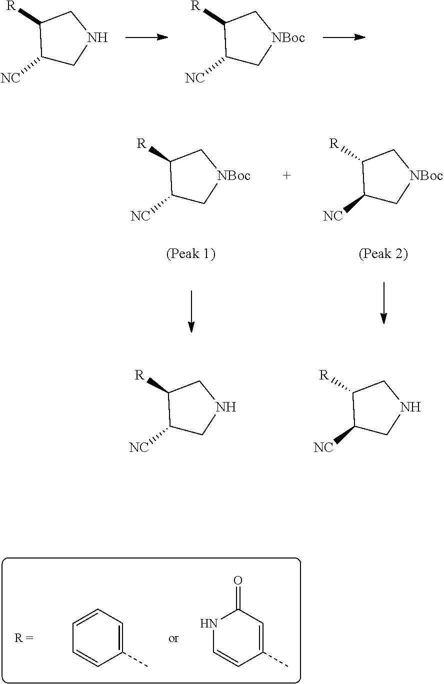

### Preparative Separation Method:

Instrument: Waters Thar 80 preparative SFC; Column: ChiralPak C-IG, 100×4.6 mm I.D., 5 m; Mobile phase: A for CO₂ and B for methanol (0.05% diethylamine); Gradient: 10% to 40% B in 8 min; Flow rate: 2.5 mL/min; Back pressure: 100 bar; Column temperature: 40° C.; Wavelength: 210 nm; Cycle-time: 2 min

TABLE 35

| Name | Structure | LCMS |
|---|---|---|
| tert-butyl (3R,4S)-3- cyano-4- phenylpyrrolidine-1- carboxylate (Peak 1) | 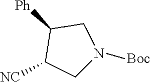 [SMILES: CC(C)(C)OC(=O)N1C[C@H](c2ccccc2)[C@@H](C#N)C1] | 217 [(M − 56) + 1]⁺ |
| tert-butyl (3S,4R)- 3-cyano-4- phenylpyrrolidine-1- carboxylate (Peak 2) | 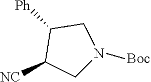 [SMILES: CC(C)(C)OC(=O)N1C[C@@H](C#N)[C@H](c2ccccc2)C1] | 217 [(M − 56) + 1]⁺ |
| tert-butyl (3R,4S)- 3-cyano-4-(2-oxo-1,2- dihydropyridin-4-yl) pyrrolidine-1-carboxylate (Peak 1) | 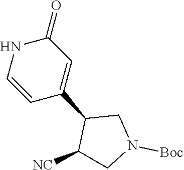 [SMILES: CC(C)(C)OC(=O)N1C[C@@H](C#N)[C@@H](c2cc[nH]c(=O)c2)C1] | 190 [M + H]⁺ |
| tert-butyl (3S,4R)- 3-cyano- 4-(2-oxo-1,2- dihydropyridin-4-yl) pyrrolidine-1-carboxylate (Peak 2) | 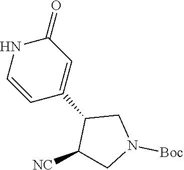 [SMILES: CC(C)(C)OC(=O)N1C[C@@H](C#N)[C@H](c2cc[nH]c(=O)c2)C1] | 190 [M + H]⁺ |

### Separation of 2-oxospiro[indoline-3,3′-pyrrolidine]-4′-carbonitrile

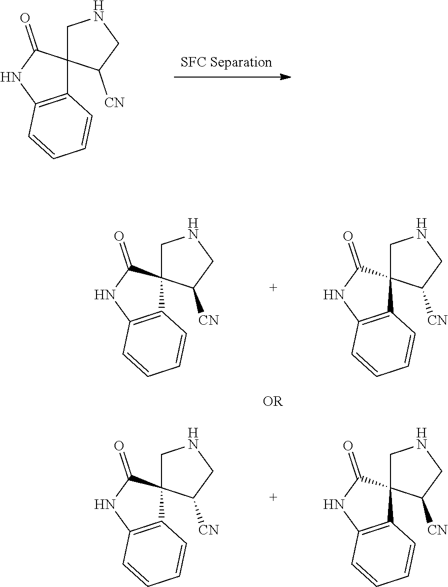 [SMILES: N#CC1CNCC12C(=O)Nc1ccccc12 ; N#C[C@@H]1CNC[C@]12C(=O)Nc1ccccc12 ; N#C[C@H]1CNC[C@@]12C(=O)Nc1ccccc12 ; N#C[C@H]1CNC[C@]12C(=O)Nc1ccccc12 ; N#C[C@@H]1CNC[C@@]12C(=O)Nc1ccccc12]

2-oxospiro[indoline-3,3′-pyrrolidine]-4′-carbonitrile (1.00 g, single diastereomer with relative stereochemistry unknown, CAS #1423027-48-8) was purchased and separated under SFC conditions (Column Lux-Cellulose-5 21.2×250 mm, 5 um column, 3.7 mg/inj, concentration 36.9 mg/mL, Column T=40° C., Flow rate 75 mL/min, 15% MeOH (with 0.1% diethylamine), cycle time: 4.9 min) to give Peak 1 or Peak 2.

Peak 1: (3S,4′S)-2-oxospiro[indoline-3,3′-pyrrolidine]-4′-carbonitrile and (3R,4′R)-2-oxospiro[indoline-3,3′-pyrrolidine]-4′-carbonitrile (230 mg each enantiomer) or (3S,4′R)-2-oxospiro[indoline-3,3′-pyrrolidine]-4′-carbonitrile or (3R,4′S)-2-oxospiro[indoline-3,3′-pyrrolidine]-4′-carbonitrile (220 mg each enantiomer)

Peak 2: (3S,4′S)-2-oxospiro[indoline-3,3′-pyrrolidine]-4′-carbonitrile and (3R,4′R)-2-oxospiro[indoline-3,3′-pyrrolidine]-4′-carbonitrile (220 mg each enantiomer) or (3S,4′R)-2-oxospiro[indoline-3,3′-pyrrolidine]-4′-carbonitrile or (3R,4′S)-2-oxospiro[indoline-3,3′-pyrrolidine]-4′-carbonitrile (220 mg each enantiomer)

### Synthesis of 6-phenyl-4-azaspiro[2.4]heptane

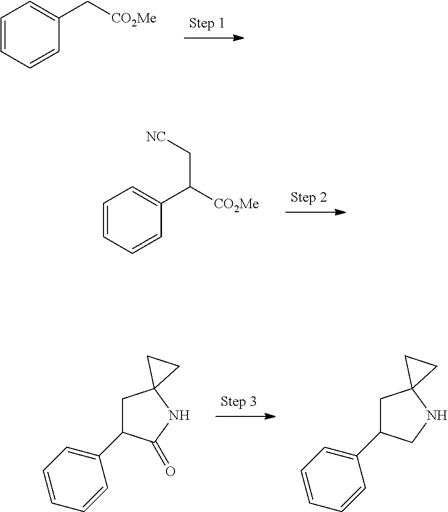 [SMILES: COC(=O)Cc1ccccc1 ; COC(=O)C(CC#N)c1ccccc1 ; O=C1NC2(CC2)CC1c1ccccc1 ; c1ccc(C2CNC3(CC3)C2)cc1]

### Step 1: Preparation of Methyl 3-cyano-2-phenylpropanoate

To a cooled (−78° C.) solution of methyl 2-phenylacetate (5.0 g, 33.3 mmol, 1.0 eq) in dry THF (50 mL) was slowly added a solution of 2 M LDA (20 mL, 40.0 mmol, 1.2 eq) in THF. After 1 h, 2-bromoacetonitrile (4.2 g, 35.0 mmol, 1.1 eq) slowly in a dropwise manner and the reaction was further aged for additional 1 h at −78° C. To the mixture was added saturated aqueous NH₄Cl (5 mL) and the mixture was warmed to room temperature. The mixture was with EtOAc (20 mL×3). The organic layers were combined, washed with brine (150 mL), dried over anhydrous Na₂SO₄, and concentrated under reduced pressure. The residue was purified by flash column chromatography on silica gel to methyl 3-cyano-2-phenylpropanoate (5.0 g, 26.5 mmol, 79%) as a white solid. LCMS (ESI) m/z=190.1 [M+H]⁺.

### Step 2: Preparation of 6-phenyl-4-azaspiro[2.4]heptan-5-one

To a cooled (0° C.) solution of methyl 3-cyano-2-phenylpropanoate (5.0 g, 26.5 mmol, 1.0 eq) and Ti(OiPr)₄ (9.0 g, 31.7 mmol, 1.2 eq) in dry THF (100 mL) was slowly added a 3 M solution of EtMgBr (20 mL, 59.6 mmol, 2.25 eq), while maintaining inner temperature between −5° C. to 0° C. After complete addition of EtMgBr, the mixture was stirred at 0° C. for additional 1 h. The reaction mixture was quenched by addition of aqueous 2 N HCl (60 mL). The resulting acidic solution was extracted with EtOAc (50 mL×3). The combined organic layers were washed with brine (20 mL×2), dried over anhydrous Na₂SO₄, and concentrated under reduced pressure. The residue was purified by flash column chromatography on to afford 6-phenyl-4-azaspiro[2.4]heptan-5-one (2.1 g, 11.2 mmol, 42% yield) as a white solid. LCMS (ESI) m/z=188 [M+H]⁺; ¹H NMR (400 MHz, CDCl₃) δ 7.39-7.24 (m, 6H), 3.86 (dd, J=9.4, 7.6 Hz, 1H), 2.51 (dd, J=12.9, 9.4 Hz, 1H), 2.29 (dd, J=12.9, 7.6 Hz, 1H), 0.85-0.88 (m, 1H), 0.87-0.81 (m, 1H), 0.76-0.64 (m, 2H).

### Step 3: Preparation of 6-phenyl-4-azaspiro[2.4]heptane

To a cooled (10° C.) solution of 6-phenyl-4-azaspiro[2.4]heptan-5-one (2.1 g, 11.2 mmol, 1.0 eq) in dry THF (50 mL) was added NaBH₄ (2.1 g, 56.0 mmol, 5.0 eq) in several portions. To the mixture was added in a dropwise manner BF₃·Et₂O (6.7 mL, 56.0 mmol, 5.0 eq). The reaction mixture was heated at 60° C. for 16 h. The mixture was cooled to ambient temperature and aqueous 2 N HCl (30 mL) was introduced slowly. The acidic mixture was extracted with EtOAc (30 mL×3). The organic layers were combined, washed with brine (20 mL), dried over anhydrous Na₂SO₄, and concentrated under reduced pressure. The residue was purified by Biotage® C18 column to afford 6-phenyl-4-azaspiro[2.4]heptane (1.5 g, 8.7 mmol, 78%) as a colorless oil. LCMS (ESI) m/z=174 [M+H]⁺; ¹H NMR (400 MHz, CDCl₃) 8.59 (s, 1H), δ 7.38-7.27 (m, 5H), 3.85-3.57 (m, 2H), 3.40-3.17 (m, 1H), 2.43-2.09 (m, 2H), 1.46-1.18 (m, 2H), 0.98-0.66 (m, 2H).

### Synthesis of 3-(2-oxo-1,2-dihydropyridin-4-yl)pyrrolidin-1-ium chloride

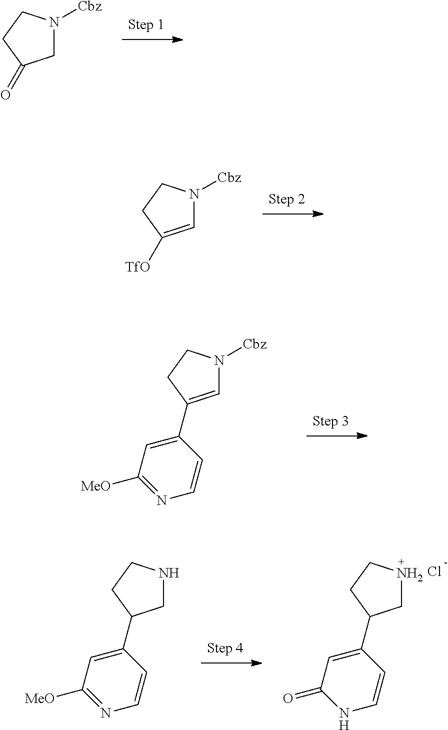 [SMILES: O=C1CCN(C(=O)OCc2ccccc2)C1 ; O=C(OCc1ccccc1)N1C=C(OS(=O)(=O)C(F)(F)F)CC1 ; COc1cc(C2=CN(C(=O)OCc3ccccc3)CC2)ccn1 ; COc1cc(C2CCNC2)ccn1 ; O=c1cc(C2CCNC2)cc[nH]1 ; Cl]

### Step 1: Preparation of Benzyl 4-(((trifluoromethyl)sulfonyl)oxy)-2,3-dihydro-1H-pyrrole-1-carboxylate

To a cooled (−78° C.) solution of benzyl 3-oxopyrrolidine-1-carboxylate (5.0 g, 22.8 mmol, 1 eq) in dry THF (80 mL) was slowly added a solution of LDA (25.0 mmol, 1.1 eq) in THF. The mixture was stirred for additional 30 min, followed by addition of a solution of 1,1,1-trifluoro-N-phenyl-N-((trifluoromethyl)sulfonyl)methanesulfonamide (8.93 g, 25.0 mmol, 1.1 eq) in THF (70 mL). After complete addition of 1,1,1-trifluoro-N-phenyl-N-((trifluoromethyl)sulfonyl)methanesulfonamide, the reaction mixture was allowed to warm to room temperature and stirred for 14 h. The reaction mixture was quenched by adding H₂O (20 mL) and extracted with EtOAc (150 mL×3). The organic layers were combined, washed with brine (150 mL), dried over anhydrous Na₂SO₄, and concentrated under reduced pressure. The residue was purified by flash column chromatography on silica gel to afford benzyl 4-(((trifluoromethyl)sulfonyl)oxy)-2,3-dihydro-1H-pyrrole-1-carboxylate (3.00 g, 8.53 mmol, 37.5% yield) as a yellow oil. LCMS (ESI) m/z=352 [M+H]⁺.

### Step 2: Preparation of Benzyl 4-(2-methoxypyridin-4-yl)-2,3-dihydro-1H-pyrrole-1-carboxylate

A solution of benzyl 4-(((trifluoromethyl)sulfonyl)oxy)-2,3-dihydro-1H-pyrrole-1-carboxylate (3.0 g, 8.53 mmol, 1.0 eq), (2-methoxypyridin-4-yl)boronic acid (1.55 g, 10.2 mmol, 1.2 eq), and K₂CO₃ (2.94 g, 21.3 mmol, 2.5 eq) in a mixture of toluene (60.0 mL), EtOH (15.0 mL) and H₂O (15.0 mL) was added Pd(PPh₃)₄(492 mg, 426 μmol, 0.05 eq). The reaction mixture was subsequently heated at 100° C. under N₂ (g) for 12 h. The reaction mixture was cooled to room temperature, diluted with H₂O (40 mL), and extracted with EtOAc (100 mL×3). The organic layers were combined, washed with brine (100 mL), dried over anhydrous Na₂SO₄, and concentrated under reduced pressure. The residue was purified by flash column chromatography on silica gel to afford benzyl 4-(2-methoxypyridin-4-yl)-2,3-dihydro-1H-pyrrole-1-carboxylate (750 mg, 2.41 mmol, 28.4% yield) as a white solid. LCMS (ESI) m/z=311 [M+H]⁺.

### Step 3: Preparation of 2-methoxy-4-(pyrrolidin-3-yl)pyridine

To a solution of benzyl 4-(2-methoxypyridin-4-yl)-2,3-dihydro-1H-pyrrole-1-carboxylate (750 mg, 2.41 mmol, 1 eq) in MeOH (50 mL) under N₂ (g) was added 10% Pd/C (100 mg). The suspension was subjected to three cycles to evacuation and purging with H₂ (g). The resulting mixture was stirred at room temperature for 14 h under H₂ (g) (1 atm). The reaction mixture was subsequently subjected to three cycles to evacuation and purging with N₂ (g) and the suspension was filtered through a pad of Celite®. The filter cake was carefully washed with MeOH (20 mL) and the combined filtrate was concentrated to dryness to give 2-methoxy-4-(pyrrolidin-3-yl)pyridine (375 mg, 2.10 mmol, 87.4% yield) as off white solid. LCMS (ESI) m/z=179 [M+H]⁺.

### Step 4: Preparation of 3-(2-oxo-1,2-dihydropyridin-4-yl)pyrrolidin-1-ium chloride

A solution of 2-methoxy-4-(pyrrolidin-3-yl)pyridine (375 mg, 2.10 mmol, 1 eq) in a solution of 1 N HCl (4 mL) 1,4-dioxane was heated and stirred at 100° C. in a sealed tube. After 24 h, the reaction mixture was concentrated under reduced pressure to afford 3-(2-oxo-1,2-dihydropyridin-4-yl)pyrrolidin-1-ium chloride (400 mg, HCl salt) as a white solid, which was used directly without further purification. LCMS (ESI) m/z=165 [M+H]⁺.

Representative Procedure for Direct Coupling of Perfluorophenyl or p-Nitrophenyl Activated Linker Esters with Amino Acid Cores for Construction of Analogues

### S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))dibutanethioate (125)

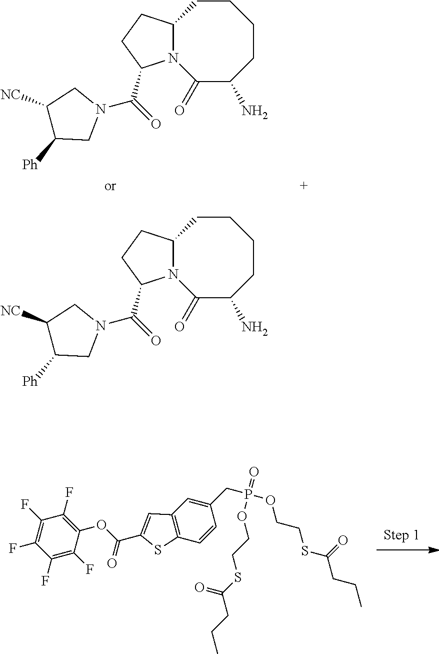 [SMILES: N#C[C@@H]1CN(C(=O)[C@@H]2CC[C@@H]3CCCC[C@H](N)C(=O)N32)C[C@H]1c1ccccc1 ; N#C[C@H]1CN(C(=O)[C@@H]2CC[C@@H]3CCCC[C@H](N)C(=O)N32)C[C@@H]1c1ccccc1 ; CCCC(=O)SCCOP(=O)(Cc1ccc2sc(C(=O)Oc3c(F)c(F)c(F)c(F)c3F)cc2c1)OCCSC(=O)CCC]

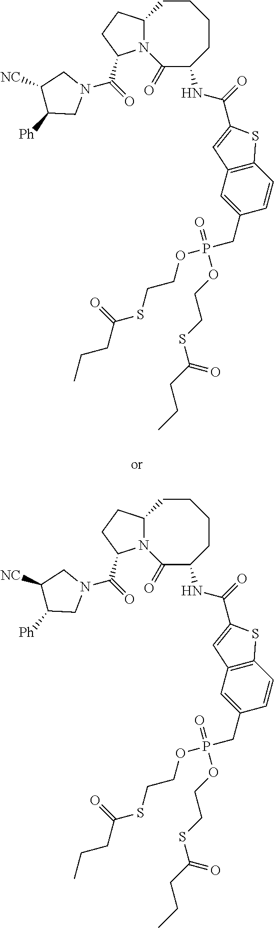 [SMILES: CCCC(=O)SCCOP(=O)(Cc1ccc2sc(C(=O)N[C@H]3CCCC[C@H]4CC[C@@H](C(=O)N5C[C@@H](C#N)[C@H](c6ccccc6)C5)N4C3=O)cc2c1)OCCSC(=O)CCC ; CCCC(=O)SCCOP(=O)(Cc1ccc2sc(C(=O)N[C@H]3CCCC[C@H]4CC[C@@H](C(=O)N5C[C@H](c6ccccc6)[C@@H](C#N)C5)N4C3=O)cc2c1)OCCSC(=O)CCC]

### Step 1: Preparation of S,S′-(((((2-(((3S,6S,10aS)-3-((3S,4R)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))dibutanethioate

To a solution of perfluorophenyl 5-((bis(2-(butyrylthio)ethoxy)phosphoryl)methyl)benzo[b]thiophene-2-carboxylate (30 mg, 42.9 μmol, 1.0 eq) and (3S,4R or 3R,4S)-1-((3S,6S,10aS)-6-amino-5-oxodecahydropyrrolo[1,2-a]azocine-3-carbonyl)-4-phenylpyrrolidine-3-carbonitrile (16 mg, 42.0 μmol, 1.0 eq) (pyrrolidine SFC peak 2 used) in CH₂Cl₂ (2 mL) was added N,N-diisopropylethylamine (10.8 mg, 84 μmol, 2.0 eq) at room temperature. After stirring for 48 h, the reaction mixture was concentrated under reduced pressure. The residue was purified by reverse phase HPLC to give S,S′-(((((2-(((3S,6S,10aS)-3-((3S,4R or 3R, 4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))dibutanethioate (35 mg, 39 μmol, 94% yield) as a white solid. LCMS (ESI) m/z=895 [M+H]+; 1H NMR (400 MHz, CDCl3) δ 7.86-7.71 (m, 3H), 7.45-7.29 (m, 6H), 7.22-7.13 (m, 1H), 5.19-5.08 (m, 1H), 4.60-4.49 (m, 1H), 4.39-4.23 (m, 2H), 4.11-3.93 (m, 6H), 3.81-3.60 (m, 2H), 3.28 (d, JHP=21.2 Hz, 2H), 3.22-3.12 (m, 1H), 3.06 (t, J=6.3 Hz, 4H), 2.52 (t, J=7.4 Hz, 4H), 2.29-1.58 (m, 16H), 0.94 (t, J=8.0 Hz, 6H).

The following compounds in Table 36 were prepared using the representative protocol described above for the synthesis S,S′-(((((2-(((3S,6S,10aS)-3-((3S,4R)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))dibutanethioate and utilizing the appropriate starting materials and modifications.

TABLE 36

| Com- | | | | |
|---|---|---|---|---|
| pound | Name | Structure | LCMS | NMR |
| 126 | S,S′-(((((2- (((3S,6S,10aS)- 3-(rel-trans)- 3-cyano-4- phenylpyrrolidine-1- carbonyl)-5- oxodecahydro- pyrrolo[1,2-a] azocin-6- yl)carbamoyl) benzo[b]thiophen- 5- yl)difluoromethyl) phosphoryl)bis(oxy)) bis(ethane- 2,1-diyl)) dibutanethioate | 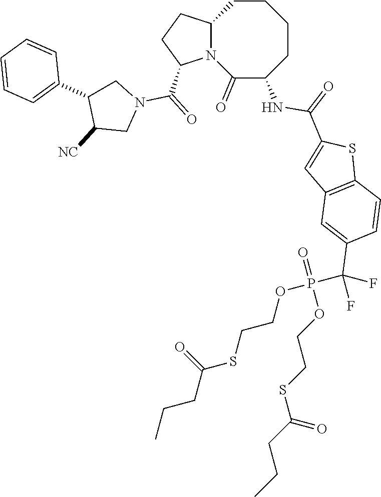 [SMILES: CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 931.7 [M + H]⁺ | ¹H NMR (400 MHz, CDCl₃) δ 8.10 (s, 1H), 7.98-7.90 (m, 1H), 7.89- 7.82 (m, 1H), 7.64 (d, J = 8.1 Hz, 1H), 7.44- 7.29 (m, 5H), 7.25-7.21 (m, 1H), 5.21-5.05 (m, 1H), 4.61-4.49 (m, 1H), 4.38-4.11 (m, 6H), 4.10- 3.96 (m, 1H), 3.92- 3.04 (m, 8H), 2.53 (t, J = 7.4 Hz, 4H), 2.31-1.76 (m, 10H), 1.74-1.65 (m, 4H), 1.44-1.23 (m, 2H), 0.94 (t, J = 7.4 Hz, 6H) |
| 127 | S,S′-(((((2- (((3S,6S,10aS)-3- ((3S,4R or 3R,4S)-3- cyano-4- phenylpyrrolidine- 1-carbonyl)-5- oxodecahydro- pyrrolo[1,2-a] azocin-6- yl)carbamoyl) benzo[b]thiophen- 5-yl) difluoromethyl) phosphoryl)bis(oxy)) bis(ethane-2,1- diyl))bis(2,2- dimethylpropane- thioate | 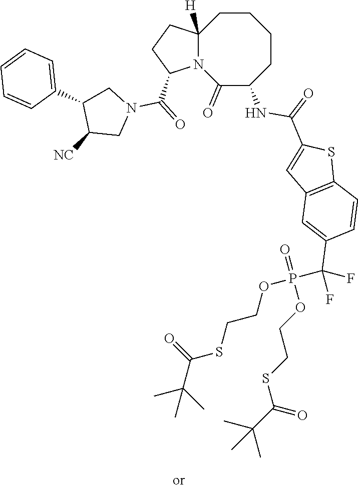 [SMILES: CC(C)(C)C(=O)SCCOP(=O)(OCCSC(=O)C(C)(C)C)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 959.1 [M + H]⁺ | ¹H NMR (400 MHz, methanol-d₄) δ 8.20 (d, J = 2.9 Hz, 1H), 8.18- 8.14 (m, 1H), 8.08 (t, J = 9.2 Hz, 1H), 7.68-7.62 (m, 1H), 7.44-7.28 (m, 5H), 5.09-5.00 (m, 1H), 4.74-4.65 (m, 1H), 4.60- 4.50 (m, 0.5H), 4.50- 4.38 (m, 1.5H), 4.30- 4.13 (m, 4H), 4.06 (dd, J = 11.5, 8.1 Hz, 0.5H), 3.96 (dd, J = 11.6, 7.5 Hz, 0.5H), 3.85 (t, J = 9.8 Hz, 0.5H), 3.79-3.64 (m, 1.5H), 3.64-3.50 (m, 2H), 3.20-3.08 (m, 4H), 2.41-2.18 (m, 2H), 2.11-1.93 (m, 6H), 1.92- 1.75 (m, 2H), 1.75- 1.57 (m, 2H), 1.20 (s, 18H) |
| | | 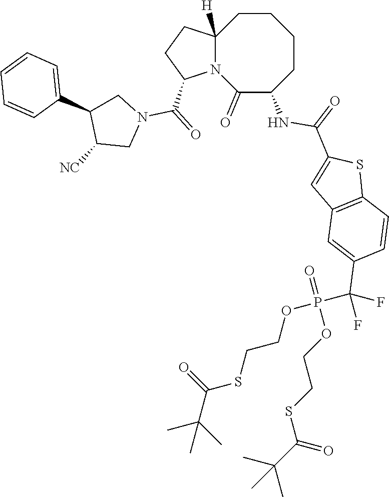 [SMILES: CC(C)(C)C(=O)SCCOP(=O)(OCCSC(=O)C(C)(C)C)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 128 | S-(2-((((2- (((3S,6S,10aS)-3- ((3R,4S or 3S,4R)- 3-cyano-4- phenylpyrrolidine- 1-carbonyl)-5- oxodecahydro- pyrrolo[1,2-a] azocin-6- yl)carbamoyl) benzo[b]thiophen- 5- yl)difluoromethyl) (phenoxy) phosphoryl)oxy) ethyl) butanethioate | 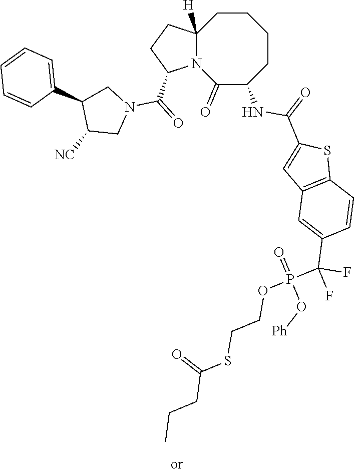 [SMILES: CCCC(=O)SCCOP(=O)(Oc1ccccc1)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | 877.1 [M + H]⁺ | ¹H NMR (400 MHz, methanol-d₄) δ 8.22- 8.17 (m, 2H), 8.14-8.07 (m, 1H), 7.72-7.67 (m, 1H), 7.47-7.27 (m, 7H), 7.27-7.20 (m, 1H), 7.15 (d, J = 7.6 Hz, 2H), 5.09- 5.00 (m, 1H), 4.69- 4.60 (m, 1H), 4.50-4.39 (m, 1H), 4.35-4.15 (m, 3H), 4.15-4.06 (m, 1H), 3.92 (t, J = 10.3 Hz, 0.5H), 3.84-3.75 (m, 0.5H), 3.68-3.59 (m, 1H), 3.52-3.38 (m, 1H), 3.17-3.10 (m, 2H), 2.52- 2.45 (m, 2H), 2.42- 2.18 (m, 2H), 2.13-1.93 (m, 6H), 1.92-1.76 (m, 2H), 1.75-1.55 (m, 4H), 1.46-1.40 (m, 1H), 0.94- 0.86 (m, 3H) |
| | | 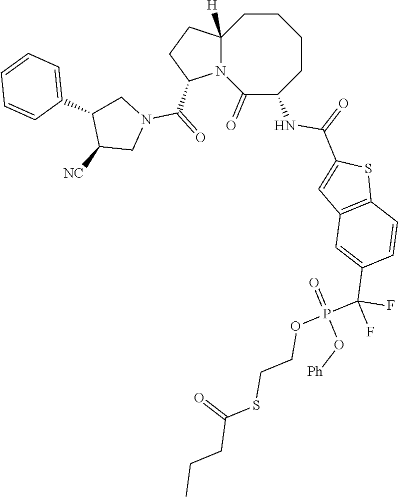 [SMILES: CCCC(=O)SCCOP(=O)(Oc1ccccc1)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | | |
| | | SFC peak 1 was used for | | |
| | | biological testing | | |
| 129 | S,S′-(((((2- (((3S,6S,8S,10aR)- 3-((3S,4R or 3R,4S)-3- cyano-4- phenylpyrrolidine- 1-carbonyl)-8- hydroxy-5- oxodecahydro- pyrrolo[1,2-a] azocin-6-yl) carbamoyl)benzo [b]thiophen-5- yl)difluoromethyl) phosphoryl)bis(oxy)) bis(ethane-2,1- diyl)) bis(2,2- dimethylpropane- thioate | 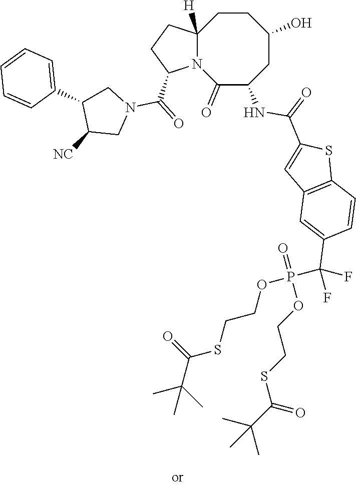 [SMILES: CC(C)(C)C(=O)SCCOP(=O)(OCCSC(=O)C(C)(C)C)C(F)(F)c1ccc2sc(C(=O)N[C@H]3C[C@@H](O)CC[C@H]4CC[C@@H](C(=O)N5C[C@@H](C#N)[C@H](c6ccccc6)C5)N4C3=O)cc2c1] | 975.2 [M + H]⁺ | ¹H NMR (400 MHz, methanol-d₄) δ 8.20- 8.18 (m, 2H), 8.12-8.06 (m, 1H), 7.67-7.65 (m, 1H), 7.42-7.28 (m, 5H), 4.99-4.96 (m, 1H), 4.73- 4.64 (m, 1H), 4.53- 4.33 (m, 2H), 4.29-4.13 (m, 4H), 4.10-3.93 (m, 2H), 3.88-3.51 (m, 4H), 3.20-3.07 (m, 4H), 2.45- 2.18 (m, 4H), 2.17- 1.73 (m, 6H), 1.20 (s, 18H) |
| | | 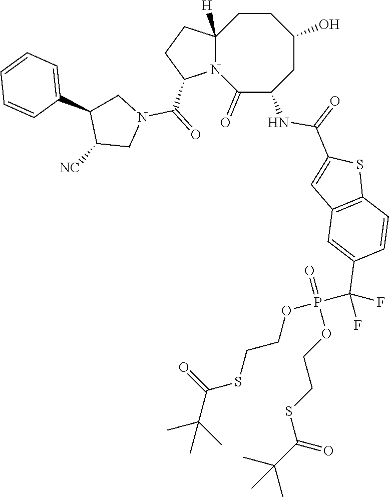 [SMILES: CC(C)(C)C(=O)SCCOP(=O)(OCCSC(=O)C(C)(C)C)C(F)(F)c1ccc2sc(C(=O)N[C@H]3C[C@@H](O)CC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 130 | S,S′- ((((difluoro(2- (((3′S,6′S,10a′S)-5′- oxo-3′-(3-(pyridin- 3-yl)azetidine-1- carbonyl)octahydro- 5′H-spiro [cyclopropane-1,8′- pyrrolo[1,2-a] azocin]-6′- yl)carbamoyl) benzo[b] thiophen-5- yl)methyl) phosphoryl)bis (oxy))bis (ethane-2,1-diyl)) bis(2,2- dimethylpropane- thioate) | 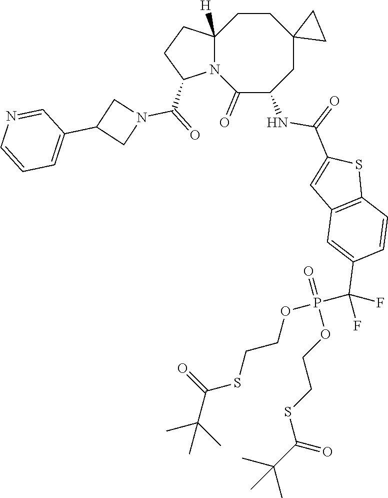 [SMILES: CC(C)(C)C(=O)SCCOP(=O)(OCCSC(=O)C(C)(C)C)C(F)(F)c1ccc2sc(C(=O)N[C@H]3CC4(CC[C@H]5CC[C@@H](C(=O)N6CC(c7cccnc7)C6)N5C3=O)CC4)cc2c1] | 947.2 [M + H]⁺ | ¹H NMR (400 MHz, methanol-d₄) δ 8.64- 8.59 (m, 0.6H), 8.56- 8.51 (m, 0.4H), 8.49- 8.42 (m, 1H), 8.17 (s, 2H), 8.12-8.02 (m, 1.6H), 7.98-7.92 (m, 0.4H), 7.69-7.62 (m, 1H), 7.51-7.41 (m, 1H), 5.08-4.97 (m, 1.6H), 4.70 (d, J = 8.1 Hz, 1H), 4.61-4.45 (m, 2.4H), 4.42 (t, J = 9.4 Hz, 0.6H), 4.34-4.12 (m, 4.4H), 4.11-3.94 (m, 2H), 3.20- 3.07 (m, 4H), 2.32- 2.16 (m, 2H), 2.15-2.03 (m, 1H), 2.02-1.83 (m, 4H), 1.78-1.57 (m, 3H), 1.19 (d, J = 5.9 Hz, 18H), 0.65-0.45 (m, 4H) |
| 131 | S,S′-(((((2- (((3S,6S,10aS)-3- (cinnolin-6- yl(methyl) carbamoyl)-5- oxodecahydro- pyrrolo[1,2-a] azocin-6- yl)carbamoyl) benzo[b]thiophen- 5-yl) difluoromethyl) phosphoryl)bis (oxy))bis(ethane- 2,1-diyl)) dibutanethioate | 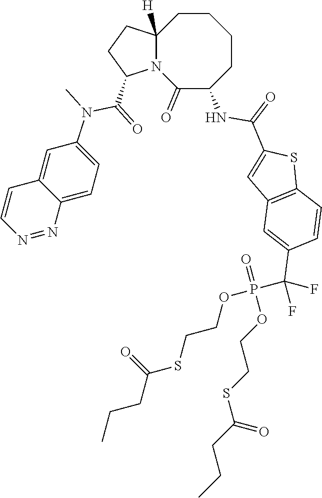 [SMILES: CCCC(=O)SCCOP(=O)(OCCSC(=O)CCC)C(F)(F)c1ccc2sc(C(=O)N[C@H]3CCCC[C@H]4CC[C@@H](C(=O)N(C)c5ccc6nnccc6c5)N4C3=O)cc2c1] | 918.1 [M + H]⁺ | ¹H NMR (400 MHz, methanol-d₄) δ 9.25 (d, J = 5.9 Hz, 1H), 8.49 (d, J = 9.0 Hz, 1H), 8.15- 8.00 (m, 6H), 7.62 (d, J = 8.6 Hz, 1H), 5.04-4.98 (m, 1H), 4.61-4.33 (m, 2H), 4.32-4.16 (m, 4H), 3.48-3.38 (m, 3H), 3.24- 3.13 (m, 4H), 2.56- 2.52 (m, 4H), 2.20-1.97 (m, 8H), 1.88-1.75 (m, 2H), 1.70-1.58 (m, 6H), 0.92-0.90 (m, 6H) |
| 132 | S,S′- ((((difluoro(2- (((3′S,6′S,10a′R)- 5′-oxo-3′- (3-(pyridin-3- yl)azetidine-1- carbonyl)octahydro- 1′H-spiro [cyclopropane-1,9′- pyrrolo[1,2-a] azocin]-6′- yl)carbamoyl) benzo[b]thiophen- 5- yl)methyl) phosphoryl)bis (oxy))bis(ethane- 2,1-diyl))bis(2,2- dimethylpropane- thioate) | 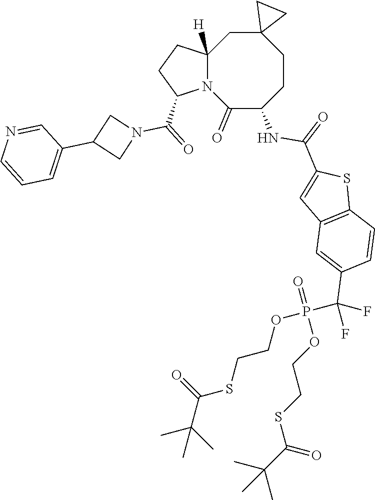 [SMILES: CC(C)(C)C(=O)SCCOP(=O)(OCCSC(=O)C(C)(C)C)C(F)(F)c1ccc2sc(C(=O)N[C@H]3CCC4(CC4)C[C@H]4CC[C@@H](C(=O)N5CC(c6cccnc6)C5)N4C3=O)cc2c1] | 947.2 [M + H]⁺ | ¹H NMR (400 MHz, methanol-d₄) δ 8.61- 8.52 (m, 1H), 8.50-8.40 (m, 1H), 8.25-8.20 (m, 1H), 8.19-8.15 (m, 1H), 8.14-7.91 (m, 2H), 7.70- 7.61 (m, 1H), 7.52- 7.39 (m, 1H), 5.23-5.14 (m, 1H), 5.02-4.97 and 4.73-4.39 (m, 5H), 4.33- 4.14 (m, 4H), 4.10- 3.95 (m, 2H), 3.23-3.03 (m, 4H), 2.72-2.49 (m, 2H), 2.38-2.15 (m, 2H), 2.13-1.98 (m, 1H), 1.97- 1.71 (m, 3H), 1.37- 1.25 (m, 1H), 1.23-1.14 (m, 18H), 0.82-0.74 (m, 1H), 0.73-0.65 (m, 1H), 0.60-0.47 (m, 2H), 0.44- 0.33 (m, 1H) |
| 133 | S-(2-((((2- (((3S,6S,10aS)-3- ((3S,4R or 3R,4S)- 3-cyano-4- phenylpyrrolidine-1- carbonyl)-5- oxodecahydro- pyrrolo[1,2-a] azocin-6- yl)carbamoyl) benzo[b]thiophen- 5- yl)difluoromethyl) (phenoxy) phosphoryl)oxy) ethyl)-2,2- dimethylpropane- thioate | 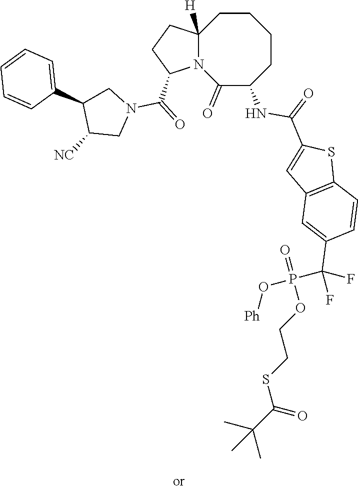 [SMILES: CC(C)(C)C(=O)SCCOP(=O)(Oc1ccccc1)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | 891.1 [M + H]⁺ | ¹H NMR (400 MHz, methanol-d₄) δ 8.22- 8.18 (m, 2H), 8.11-8.07 (m, 1H), 7.71-7.68 (m, 1H), 7.42-7.31 (m, 7H), 7.26-7.13 (m, 3H), 5.08- 5.00 (m, 1H), 4.73- 4.65 (m, 1H), 4.56-4.39 (m, 2H), 4.34-4.19 (m, 2H), 4.10-3.93 (m, 1H), 3.88-3.50 (m, 4H), 3.11- 3.08 (m, 2H), 2.41- 2.18 (m, 2H), 2.11-1.94 (m, 6H), 1.92-1.77 (m, 2H), 1.73-1.59 (m, 2H), 1.16 (s, 9H) |
| | | 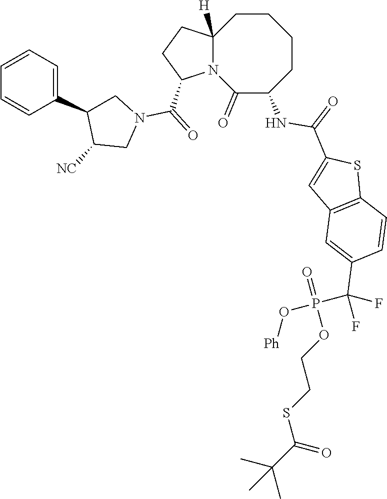 [SMILES: CC(C)(C)C(=O)SCCOP(=O)(Oc1ccccc1)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 134 | S-(2-((((2- (((3S,6S,10aS)-3- ((3S,4R or 3R,4S)-3- cyano-4- phenylpyrrolidine- 1- carbonyl)-5- oxodecahydro- pyrrolo[1,2- a]azocin-6- yl)carbamoyl) benzo[b]thiophen- 5- yl)difluoromethyl)(4- cyanophenoxy) phosphoryl)oxy) ethyl)-2,2- dimethylpropane- thioate | 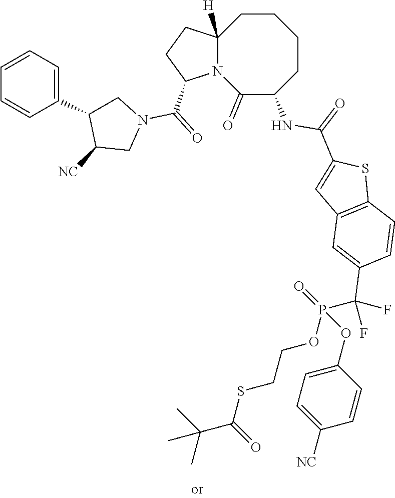 [SMILES: CC(C)(C)C(=O)SCCOP(=O)(Oc1ccc(C#N)cc1)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 916.2 [M + H]⁺ | ¹H NMR (400 MHz, methanol-d₄) δ 8.21- 8.17 (m, 2H), 8.13-8.08 (m, 1H), 7.76 (d, J = 8.6 Hz, 2H), 7.70-7.66 (m, 1H), 7.45-7.35 (m, 7H), 5.07-5.01 (m, 1H), 4.72- 4.66 (m, 1H), 4.56- 4.38 (m, 2H), 4.37-4.26 (m, 2H), 4.10-3.93 (m, 1H), 3.73-3.46 (m, 4H), 3.16-3.09 (m, 2H), 2.40- 2.19 (m, 2H), 2.08- 1.93 (m, 6H), 1.91-1.79 (m, 2H), 1.74-1.62 (m, 2H), 1.15 (s, 9H) |
| | | 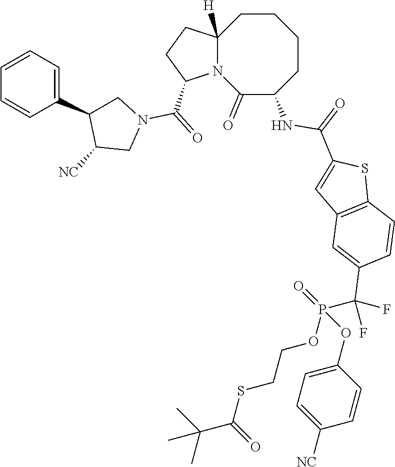 [SMILES: CC(C)(C)C(=O)SCCOP(=O)(Oc1ccc(C#N)cc1)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 135 | S,S′-(((((2- (((3S,6S,8S,10aR)- 3-((3S,4R or 3R,4S)-3- cyano-4- phenylpyrrolidine- 1- carbonyl)-8- hydroxy-5- oxodecahydro- pyrrolo[1,2- a]azocin-6- yl)carbamoyl) benzo[b]thiophen- 5- yl)difluoromethyl) phosphoryl) bis(oxy))bis (ethane-2,1-diyl)) bis(3- methylbutane- thioate) | 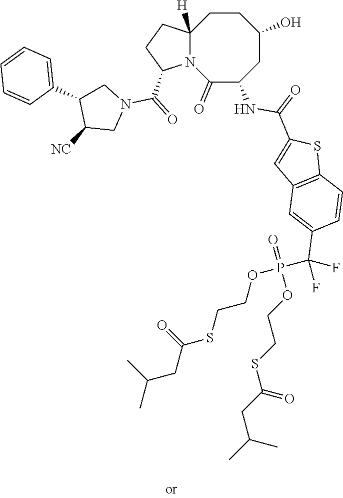 [SMILES: CC(C)CC(=O)SCCOP(=O)(OCCSC(=O)CC(C)C)C(F)(F)c1ccc2sc(C(=O)N[C@H]3C[C@@H](O)CC[C@H]4CC[C@@H](C(=O)N5C[C@@H](C#N)[C@H](c6ccccc6)C5)N4C3=O)cc2c1] | 975.0 [M + H]⁺ | ¹H NMR (400 MHz, methanol-d₄) δ 8.20- 8.17 (m, 2H), 8.11-8.08 (m, 1H), 7.66-7.64 (m, 1H), 7.44-7.29 (m, 5H), 4.99-4.96 (m, 1H), 4.73- 4.64 (m, 1H), 4.61- 4.35 (m, 2H), 4.30-4.15 (m, 4H), 4.11-3.94 (m, 2H), 3.88-3.50 (m, 4H), 3.20-3.13 (m, 4H), 2.45- 2.41 (m, 4H), 2.40- 2.15 (m, 4H), 2.14-1.84 (m, 7H), 1.83-1.74 (m, 1H), 0.94-0.91 (m, 12H) |
| | | 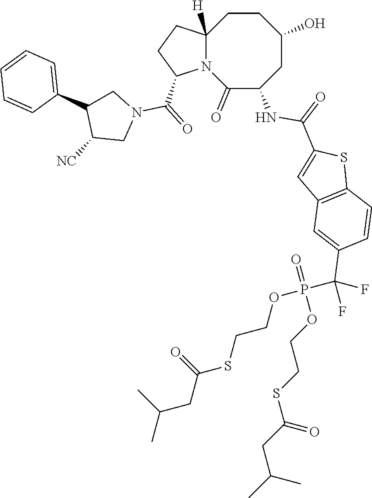 | | |
| | | SFC peak 2 was used for | | |
| | | biological testing | | |
| 136 | S-(2- (((difluoro(2- (((3S,6S,9aS)-5- oxo-3-(3- (pyridin-3- yl)azetidine-1- carbonyl) octahydro-1H- pyrrolo[1,2- a]azepin-6- yl)carbamoyl) benzo[b]thiophen- 5-yl)methyl) (phenoxy) phosphoryl)oxy) ethyl) butanethioate | 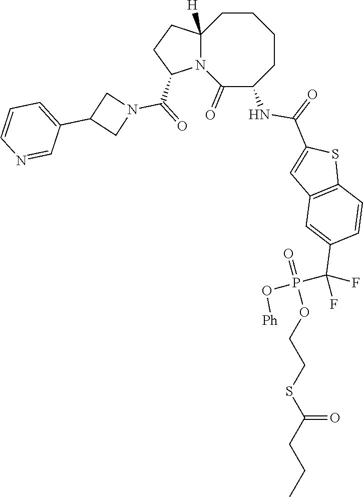 | 825.1 [M + H]⁺ | ¹H NMR (400 MHz, methanol-d₄) δ 8.59- 5.54 (m, 1H), 8.47-8.41 (m, 1H), 8.25-8.17 (m, 2H), 8.13-7.93 (m, 2H), 7.70 (d, J = 8.6 Hz, 1H), 7.50-7.32 (m, 3H), 7.27- 7.20 (m, 1H), 7.16- 7.14 (m, 2H), 4.79-4.75 (m, 1H), 4.73-4.67 (m, 1H), 4.62-4.50 (m, 2H), 4.49-4.36 (m, 1H), 4.34- 4.23 (m, 2H), 4.11- 3.97 (m, 3H), 3.14 (t, J = 6.2 Hz, 2H), 2.53-2.44 (m, 2H), 2.37-2.26 (m, 1H), 2.20-2.06 (m, 2H), 2.05-1.81 (m, 7H), 1.67- 1.54 (m, 2H), 0.93- 0.86 (m, 3H) |
| 137 | S-(2-((((2- (((3S,6S,10aS)-3- ((3S,4R or 3R,4S)- 3-cyano-4- phenylpyrrolidine-1- carbonyl)-5- oxodecahydro- pyrrolo[1,2-a] azocin-6- yl)carbamoyl) benzo[b]thiophen- 5-yl) difluoromethyl)(3- cyanophenoxy) phosphoryl)oxy) ethyl)-2,2- dimethylpropane- thioate | 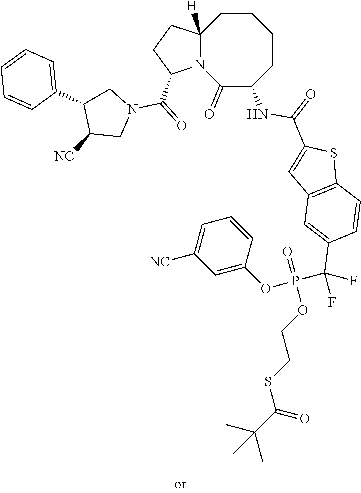 [SMILES: CC(C)(C)C(=O)SCCOP(=O)(Oc1cccc(C#N)c1)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 916.0 [M + H]⁺ | ¹H NMR (400 MHz, CD₃CN) δ 8.20-8.13 (m, 1H), 8.07-7.94 (m, 2H), 7.79-7.60 (m, 2H), 7.60-7.55 (m, 1H), 7.54- 7.43 (m, 3H), 7.42- 7.28 (m, 5H), 5.08-4.99 (m, 1H), 4.61-4.57 (m, 1H), 4.40-4.19 (m, 4H), 4.10-3.92 (m, 1H), 3.82- 3.32 (m, 4H), 3.16- 3.04 (m, 2H), 2.35-2.20 (m, 1H), 2.13-1.96 (m, 5H), 1.89-1.60 (m, 6H), 1.15 (s, 9H) |
| | | 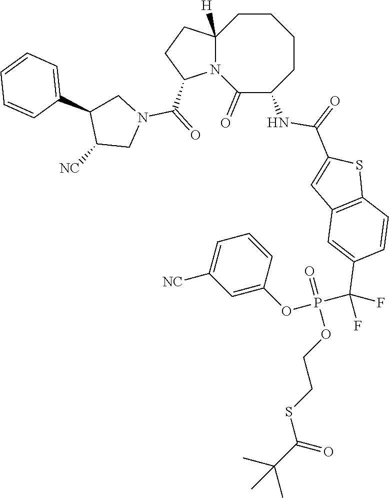 [SMILES: CC(C)(C)C(=O)SCCOP(=O)(Oc1cccc(C#N)c1)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 138 | S-(2-((((2- (((3S,6S,10aS)- 3-((3S,4R or 3R,4S)- 3-cyano-4- phenylpyrrolidine-1- carbonyl)-5- oxodecahydro- pyrrolo[1,2- a]azocin-6- yl)carbamoyl) benzo[b]thiophen- 5-yl)difluoromethyl) (3-cyanophenoxy) phosphoryl)oxy) ethyl)butane- thioate | 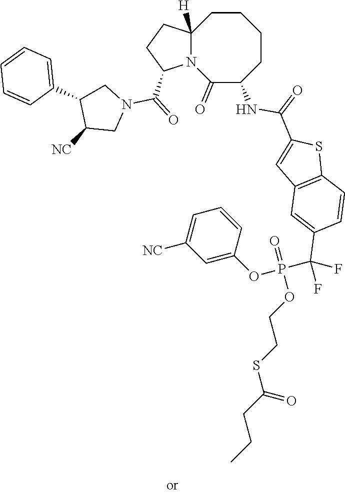 [SMILES: CCCC(=O)SCCOP(=O)(Oc1cccc(C#N)c1)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 902.1 [M + H]⁺ | ¹H NMR (400 MHz, methanol-d₄) δ 8.23- 8.18 (m, 2H), 8.12-8.07 (m, 1H), 7.72-7.65 (m, 1H), 7.63-7.46 (m, 4H), 7.42-7.27 (m, 5H), 5.05- 5.00 (m, 1H), 4.73- 4.65 (m, 1H), 4.63-4.24 (m, 5H), 4.10-3.83 (m, 1H), 3.77-3.66 (m, 1H), 3.63-3.48 (m, 2H), 3.22- 3.10 (m, 2H), 2.50 (t, J = 7.3 Hz, 2H), 2.39- 2.19 (m, 2H), 2.10-1.94 (m, 6H), 1.92-1.76 (m, 2H), 1.74-1.54 (m, 4H), 0.90 (t, J = 7.3 Hz, 3H) |
| | | 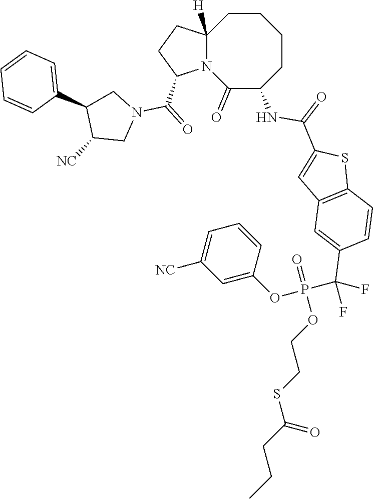 [SMILES: CCCC(=O)SCCOP(=O)(Oc1cccc(C#N)c1)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 139 | S-(2-((((2- (((3S,6S,10aS)-3- ((3S,4R or 3R,4S)- 3-cyano-4- phenylpyrrolidine-1- carbonyl)-5- oxodecahydro- pyrrolo[1,2- a]azocin-6- yl)carbamoyl) benzo[b]thiophen- 5-yl) difluoromethyl) (pyridin-3-yloxy) phosphoryl)oxy) ethyl) butanethioate | 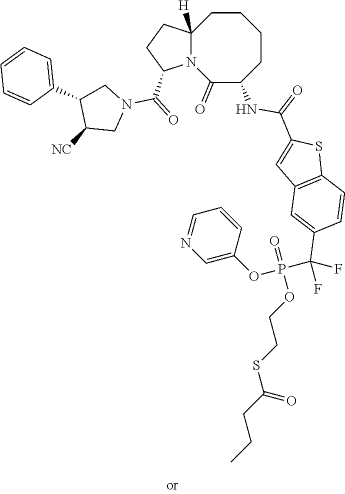 [SMILES: CCCC(=O)SCCOP(=O)(Oc1cccnc1)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 878.1 [M + H]⁺ | ¹H NMR (400 MHz, acetonitrile-d₃) δ 8.46- 8.41 (m, 2H), 8.22-8.15 (m, 1H), 8.10-8.02 (m, 1H), 8.00 (s, 1H), 7.75- 7.60 (m, 2H), 7.60-7.52 (m, 1H), 7.45-7.26 (m, 6H), 5.09-4.98 (m, 1H), 4.66-4.49 (m, 1H), 4.42- 4.18 (m, 4H), 4.12- 3.95 (m, 1H), 3.85-3.32 (m, 4H), 3.13 (t, J = 6.2 Hz, 2H), 2.49 (t, J = 7.3 Hz, 2H), 2.38-2.21 (m, 1H), 2.12-1.96 (m, 5H), 1.84-1.51 (m, 8H), 0.88 (t, J = 7.3 Hz, 3H) |
| | | 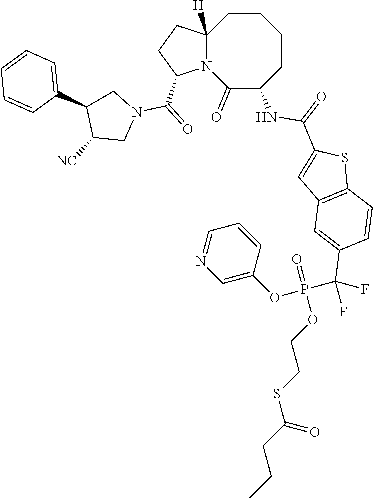 [SMILES: CCCC(=O)SCCOP(=O)(Oc1cccnc1)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 140 | S-(2- (((difluoro(2- (((3S,6S,9aS)-5- oxo-3-(3- (pyridin-3-yl) azetidine-1- carbonyl)octahydro- 1H-pyrrolo[1,2- a]azepin-6- yl)carbamoyl) benzo[b]thiophen- 5-yl)methyl) (naphthalen-1- yloxy) phosphoryl)oxy) ethyl) butanethioate | 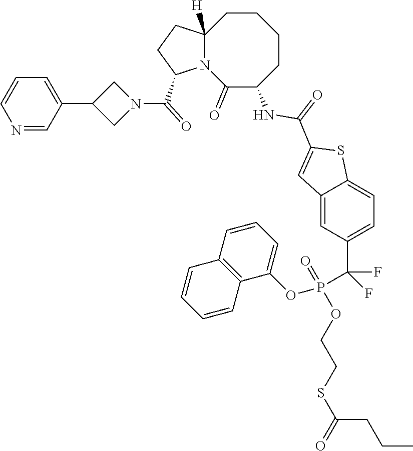 | 875.0 [M + H]⁺ | ¹H NMR (400 MHz, methanol-d₄) δ 8.57- 8.52 (m, 1H), 8.48-8.36 (m, 1H), 8.25-8.20 (m, 1H), 8.14-8.08 (m, 2H), 8.06-7.93 (m, 1H), 7.91- 7.88 (m, 1H), 7.83- 7.81 (m, 1H), 7.77-7.71 (m, 2H), 7.57-7.50 (m, 1H), 7.49-7.33 (m, 4H), 4.97-4.88 (m, 1H), 4.78- 4.67 (m, 1H), 4.62- 4.37 (m, 3H), 4.34-4.24 (m, 2H), 4.11-3.95 (m, 3H), 3.08 (t, J = 5.9 Hz, 2H), 2.43-2.37 (m, 2H), 2.35-2.26 (m, 1H), 2.17- 1.78 (m, 9H), 1.60- 1.48 (m, 2H), 0.90-0.82 (m, 3H) |
| 141 | (((difluoro(2- (((3S,6S,10aR,Z)- 3-(methyl (phenyl) carbamoyl)-5-oxo- 1,2,3,5,6,7,10,10a- octahydropyrrolo [1,2-a]azocin-6- yl)carbamoyl)-1H- indol-5-yl)methyl) phosphoryl)bis (oxy))bis (methylene) bis(2,2- dimethylpropanoate) | 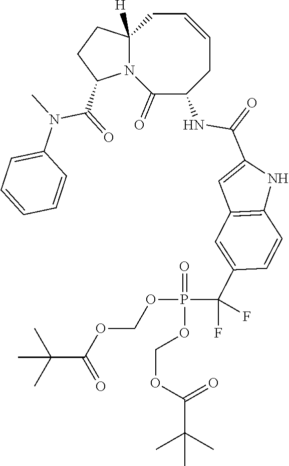 [SMILES: CN(C(=O)[C@@H]1CC[C@@H]2C/C=C\C[C@H](NC(=O)c3cc4cc(C(F)(F)P(=O)(OCOC(=O)C(C)(C)C)OCOC(=O)C(C)(C)C)ccc4[nH]3)C(=O)N21)c1ccccc1] | 815.0 [M + H]⁺ | ¹H NMR (400 MHz, CDCl₃) δ 9.77 (s, 1H), 8.68 (s, 1H), 7.75 (s, 1H), 7.40-7.22 (m, 6H), 7.16 (d, J = 8.6 Hz, 1H), 6.91 (s, 1H), 6.03-5.90 (m, 2H), 5.82-5.63 (m, 4H), 5.25-5.15 (m, 1H), 4.53- 4.46 (m, 1H), 4.30- 4.20 (m, 1H), 3.27 (s, 3H), 2.82-2.57 (m, 4H), 2.30-2.02 (m, 2H), 2.01- 1.89 (m, 2H), 1.22 (d, J = 2.7 Hz, 18H) |
| 142 | (((difluoro(2- (((3S,6S,10aS)-3- (methyl(phenyl) carbamoyl)-5- oxodecahydro- pyrrolo[1,2- a]azocin-6- yl)carbamoyl)-1H- indol-5-yl)methyl) phosphoryl)bis(oxy)) bis(methylene) bis(2,2- dimethylpropanoate) | 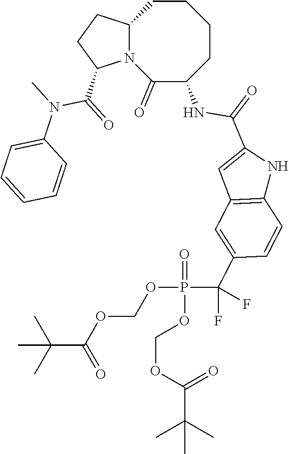 [SMILES: CN(C(=O)[C@@H]1CC[C@@H]2CCCC[C@H](NC(=O)c3cc4cc(C(F)(F)P(=O)(OCOC(=O)C(C)(C)C)OCOC(=O)C(C)(C)C)ccc4[nH]3)C(=O)N21)c1ccccc1] | 816.8 [M + H]⁺ | ¹H NMR (400 MHz, CDCl₃) δ 9.48-9.18 (m, 1H), 8.19-7.62 (m, 2H), 7.55-7.05 (m, 7H), 6.85- 6.95 (m, 1H), 5.80- 5.60 (m, 4H), 5.13-5.01 (m, 1H), 4.42-4.35 (m, 1H), 4.31-4.17 (m, 1H), 3.30 (d, J = 5.8 Hz, 3H), 2.22-1.71 (m, 12H), 1.22 (s, 18H) |
| 143 | ((((2- (((3S,6S,10aS)-3- (rel-(trans)- 3-cyano-4- cyclohexyl- pyrrolidine-1- carbonyl)-5- oxodecahydro- pyrrolo[1,2-a] azocin-6- yl)carbamoyl) benzo[b]thiophen- 5-yl) difluoromethyl) phosphoryl)bis (oxy))bis (methylene) bis (2,2-dimethyl- propanaote) | 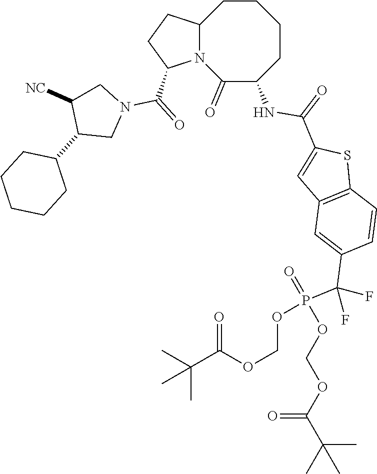 [SMILES: CC(C)(C)C(=O)OCOP(=O)(OCOC(=O)C(C)(C)C)C(F)(F)c1ccc2sc(C(=O)N[C@H]3CCCCC4CC[C@@H](C(=O)N5C[C@H](C6CCCCC6)[C@@H](C#N)C5)N4C3=O)cc2c1] | 905.3 [M + H]⁺ | ¹H NMR (400 MHz, CDCl₃) δ 9.48-9.18 (m, 1H), 8.19-7.62 (m, 2H), 7.55-7.05 (m, 7H), 6.85- 6.95 (m, 1H), 5.80- 5.60 (m, 4H), 5.13-5.01 (m, 1H), 4.42-4.35 (m, 1H), 4.31-4.17 (m, 1H), 3.30 (d, J = 5.8 Hz, 3H), 2.22-1.71 (m, 12H), 1.22 (s, 18H) |
| 144 | ((((2- (((3S,6S,10aS)-3- ((3S,4R or 3R,4S)- 3-cyano-4- phenylpyrrolidine-1- carbonyl)-5- oxodecahydro- pyrrolo[1,2- a]azocin-6- yl)carbamoyl) benzo[b]thiophen-5- yl)methyl) phosphoryl)bis (oxy))bis (methylene) bis(2,2- dimethylpropanaote) | 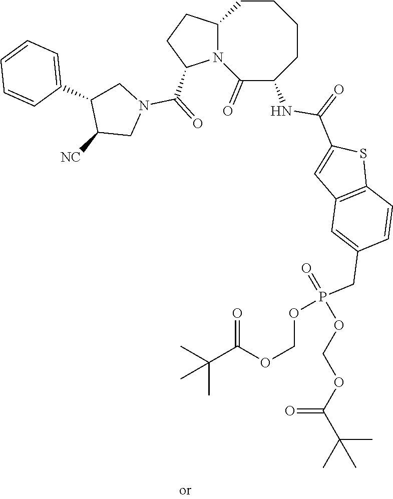 [SMILES: CC(C)(C)C(=O)OCOP(=O)(Cc1ccc2sc(C(=O)N[C@H]3CCCC[C@H]4CC[C@@H](C(=O)N5C[C@@H](C#N)[C@H](c6ccccc6)C5)N4C3=O)cc2c1)OCOC(=O)C(C)(C)C] | 863.3 [M + H]⁺ | ¹H NMR (400 MHz, DMSO-d₆) δ 7.77-7.61 (m, 3H), 7.33-7.27 (m, 8.0 Hz, 6H), 7.13-7.09 (m, 1H), 5.65-5.50 (m, 4H), 5.12-5.01 (m, 1H), 4.54-4.45 (m, 1H), 4.30- 4.22 (m, 2H), 4.06- 3.92 (m, 2H), 3.71-3.56 (m, 2H), 3.30-3.25 (m, 2H), 3.17-3.05 (m, 1H), 2.12 (d, J = 17.9 Hz, 4H), 1.83-1.54 (m, 6H), 1.49 (d, J = 6.5 Hz, 1H), 1.42 (d, J = 6.6 Hz, 1H), 1.11 (s, 18H) |
| | | 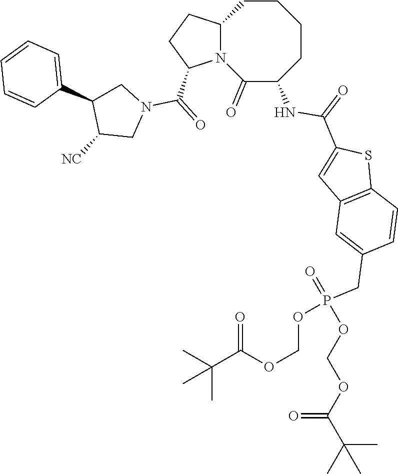 [SMILES: CC(C)(C)C(=O)OCOP(=O)(Cc1ccc2sc(C(=O)N[C@H]3CCCC[C@H]4CC[C@@H](C(=O)N5C[C@H](c6ccccc6)[C@@H](C#N)C5)N4C3=O)cc2c1)OCOC(=O)C(C)(C)C] | | |
| | | SFC peak 2 was used for | | |
| | | synthesis and biological | | |
| | | testing | | |
| 145 | ((((2- (((3S,6S,10aS)-3-(4- (N,N- dimethylsulfamoyl) piperazine-1- carbonyl)-5- oxodecahydro- pyrrolo[1,2- a]azocin-6- yl)carbamoyl) benzo[b]thiophen- 5- yl)difluoromethyl) phosphoryl)bis (oxy))bis (methylene) bis(2,2- dimethylpropanoate) | 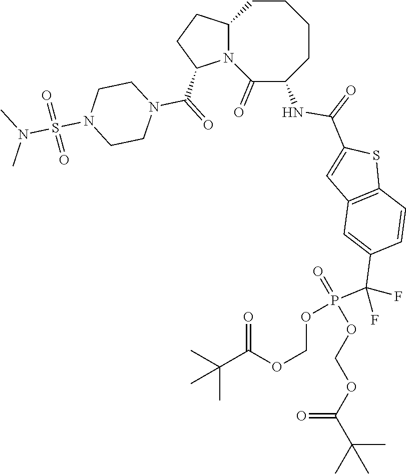 [SMILES: CN(C)S(=O)(=O)N1CCN(C(=O)[C@@H]2CC[C@@H]3CCCC[C@H](NC(=O)c4cc5cc(C(F)(F)P(=O)(OCOC(=O)C(C)(C)C)OCOC(=O)C(C)(C)C)ccc5s4)C(=O)N32)CC1] | 920.3 [M + H]⁺ | ¹H NMR (400 MHz, CDCl₃) δ 8.00 (s, 1H), 7.86 (d, J = 8.5 Hz, 1H), 7.77 (s, 1H), 7.56 (d, J = 8.4 Hz, 1H), 7.32 (d, J = 7.5 Hz, 1H), 5.68-5.55 (s, 4H), 5.13-5.01 (m, 1H), 4.79-4.68 (m, 1H), 4.33-4.20 (m, 1H), 3.86- 3.48 (m, 4H), 3.41- 3.13 (m, 4H), 2.78 (s, 6H), 2.16-1.89 (m, 8H), 1.80-1.71 (m, 2H), 1.63- 1.55 (m, 2H), 1.13 (s, 18H) |
| 146 | ((((2- (((3S,6S,11aS)-3- (rel-(trans)-3- cyano-4- phenylpyrrolidine- 1-carbonyl)-5- oxodecahydro-1H- pyrrolo[1,2-a] azonin-6- yl)carbamoyl) benzo[b]thiophen- 5-yl) difluoromethyl) phosphoryl)bis (oxy))bis(methylene) bis(2,2-dimethyl- propanoate) | 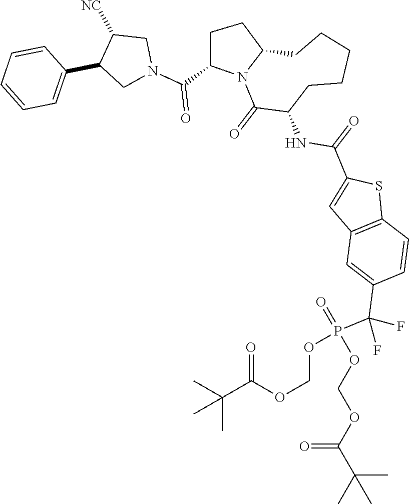 [SMILES: CC(C)(C)C(=O)OCOP(=O)(OCOC(=O)C(C)(C)C)C(F)(F)c1ccc2sc(C(=O)N[C@H]3CCCCC[C@H]4CC[C@@H](C(=O)N5C[C@@H](C#N)[C@H](c6ccccc6)C5)N4C3=O)cc2c1] | 913.3 [M + H]⁺ | ¹H NMR (400 MHz, CDCl₃) δ 8.12-8.03 (m, 1H), 7.98-7.88 (m, 1H), 7.87-7.77 (m, 1H), 7.67- 7.59 (m, 1H), 7.44- 7.28 (m, 5H), 7.02-6.80 (m, 1H), 5.77-5.69 (m, 2H), 5.69-5.61 (m, 2H), 5.38-5.25 (m, 1H), 4.66- 4.57 (m, 1H), 4.32- 4.22 (m, 1H), 4.09-3.96 (m, 1H), 3.85-3.51 (m, 2H), 3.36-3.08 (m, 1H), 2.28-2.08 (m, 5H), 2.03- 1.87 (m, 4H), 1.87- 1.82 (m, 2H), 1.79-1.73 (m, 2H), 1.70-1.55 (m, 3H), 1.20 (s, 18H) |
| 147 | ((((2- (((3S,6S,10aS)-3- (rel-trans)-3- cyano-4-(2-oxo- 1,2-dihydro- pyridin-4-yl) pyrrolidine-1- carbonyl)-5- oxodecahydro- pyrrolo[1,2-a] azocin-6- yl)carbamoyl) benzo[b]thiophen- 5-yl)difluoromethyl) phosphoryl)bis (oxy))bis (methylene) bis(2,2- dimethylpropanoate) | 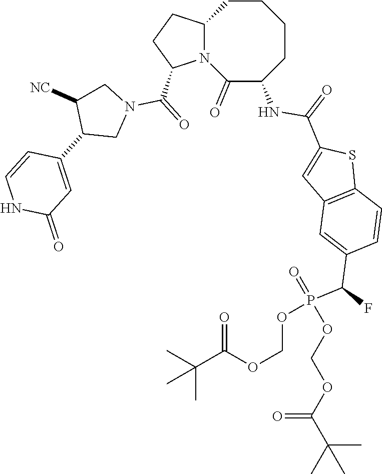 | 916.3 [M + H]⁺ | ¹H NMR (400 MHz, CDCl₃) δ 8.16-8.02 (m, 1H), 8.01-7.79 (m, 2H), 7.69-7.53 (m, 1H), 7.44- 7.29 (m, 1H), 6.74- 6.42 (m, 1H), 6.22 (s, 1H), 5.81-5.58 (m, 4H), 5.26-5.04 (m, 1H), 4.65- 4.47 (m, 1H), 4.42- 4.26 (m, 1H), 4.23-4.02 (m, 1H), 4.01-3.73 (m, 2H), 3.71-3.39 (m, 2H), 3.38-3.09 (m, 1H), 2.29- 2.06 (m, 5H), 2.03- 1.83 (m, 4H), 1.82-1.73 (m, 1H), 1.72-1.57 (m, 2H), 1.19 (s, 18H) |
| 148 | ((((2- (((3S,6S,10aS)-3- ((3S,4R or 3R,4S)-3- cyano-4- phenylpyrrolidine-1- carbonyl)-5- oxodecahydro- pyrrolo[1,2-a] azocin-6-yl)carbamoyl) benzo[b]thiophen-5- yl)methyl)phosphoryl) bis(oxy))bis(methylene) diisopropyl bis(carbonate) | 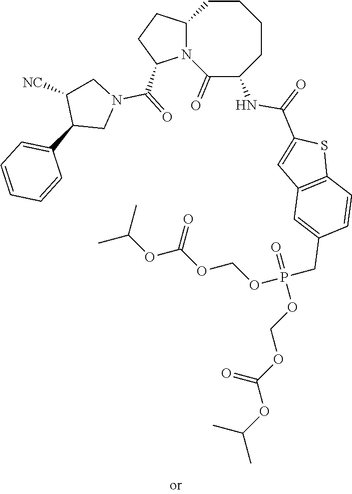 [SMILES: CC(C)OC(=O)OCOP(=O)(Cc1ccc2sc(C(=O)N[C@H]3CCCC[C@H]4CC[C@@H](C(=O)N5C[C@@H](C#N)[C@H](c6ccccc6)C5)N4C3=O)cc2c1)OCOC(=O)OC(C)C] | 867.3 [M + H]⁺ | ¹H NMR (400 MHz, CDCl₃) δ 7.79-7.75 (m, 3H), 7.41-7.30 (m, 7H), 5.68-5.54 (m, 4H), 5.21- 5.09 (m, 1H), 4.96- 4.83 (m, 2H), 4.61-4.50 (m, 1H), 4.40-4.31 (m, 2H), 4.09-3.92 (m, 2H), 3.78-3.63 (m, 2H), 3.39 (d, J = 22.4 Hz, 2H), 3.33- 3.23 (m, 1H), 2.30- 1.59 (m, 12H), 1.30 (d, J = 6.0 Hz, 12H) |
| | | 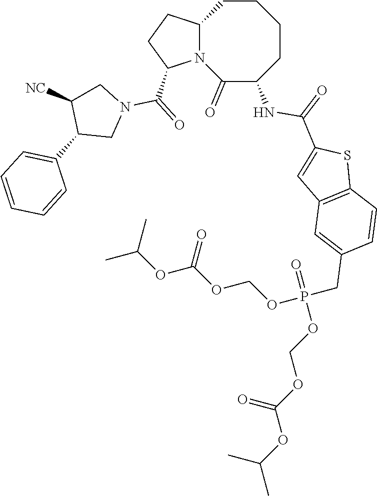 [SMILES: CC(C)OC(=O)OCOP(=O)(Cc1ccc2sc(C(=O)N[C@H]3CCCC[C@H]4CC[C@@H](C(=O)N5C[C@H](c6ccccc6)[C@@H](C#N)C5)N4C3=O)cc2c1)OCOC(=O)OC(C)C] | | |
| | | SFC peak 2 was used for | | |
| | | biological testing | | |
| 149 | S,S′-(((((2- (((3S,6S,10aS)-3- ((3S,4R or 3R,4S)-3-cyano-4- phenylpyrrolidine-1- carbonyl)-5- oxodecahydro- pyrrolo[1,2-a] azocin-6- yl)carbamoyl) benzo[b]thiophen- 5-yl)methyl) phosphoryl)bis (oxy))bis(ethane- 2,1-diyl))bis(2,2- dimethyl- propanethioate) | 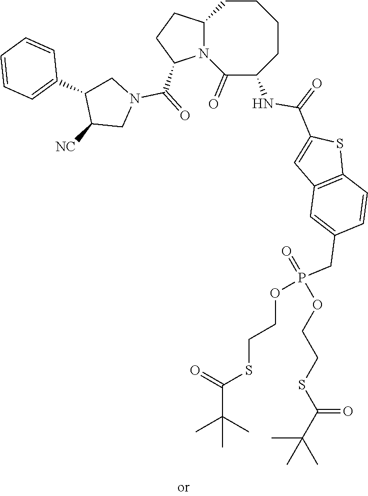 [SMILES: CC(C)(C)C(=O)SCCOP(=O)(Cc1ccc2sc(C(=O)N[C@H]3CCCC[C@H]4CC[C@@H](C(=O)N5C[C@@H](C#N)[C@H](c6ccccc6)C5)N4C3=O)cc2c1)OCCSC(=O)C(C)(C)C] | 923.4 [M + H]⁺ | ¹H NMR (400 MHz, CDCl₃) δ 7.86-7.74 (m, 3H), 7.45-7.17 (m, 8H), 5.19-5.08 (m, 1H), 4.61- 4.52 (m, 1H), 4.37- 4.26 (m, 2H), 4.11-3.95 (m, 6H), 3.76-3.67 (m, 2H), 3.36-3.20 (m, 3H), 3.09-3.00 (m, 4H), 2.25- 1.87 (m, 10H), 1.75- 1.66 (m, 2H), 1.22 (s, 18H) |
| | | 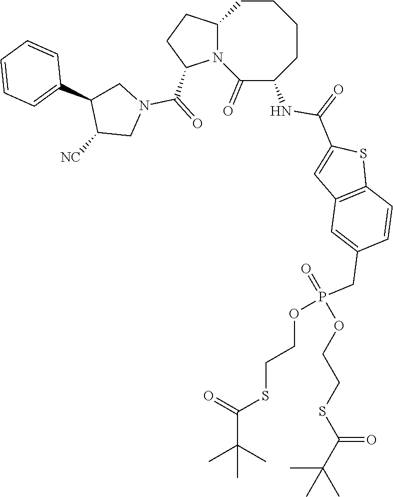 [SMILES: CC(C)(C)C(=O)SCCOP(=O)(Cc1ccc2sc(C(=O)N[C@H]3CCCC[C@H]4CC[C@@H](C(=O)N5C[C@H](c6ccccc6)[C@@H](C#N)C5)N4C3=O)cc2c1)OCCSC(=O)C(C)(C)C] | | |
| | | SFC peak 2 was used for | | |
| | | biological testing | | |
| 150 | (((difluoro(2- (((3S,6S,10aS)-6- methyl-5-oxo-3-(3- (pyridin-2-yl) azetidine-1- carbonyl) decahydropyrrolo [1,2-a]azocin-6- yl)carbamoyl) benzo[b]thiophen-5- yl)methyl) phosphoryl)bis (oxy))bis (methylene) bis(2,2-dimethyl- propanoate) | 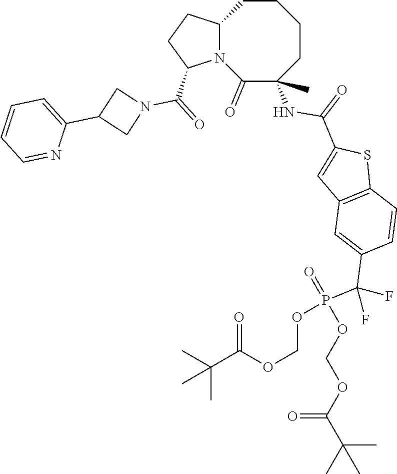 [SMILES: CC(C)(C)C(=O)OCOP(=O)(OCOC(=O)C(C)(C)C)C(F)(F)c1ccc2sc(C(=O)N[C@@]3(C)CCCC[C@H]4CC[C@@H](C(=O)N5CC(c6ccccn6)C5)N4C3=O)cc2c1] | 875.0 [M + H]⁺ | ¹H NMR (400 MHz, CDCl₃) δ 8.73-8.47 (m, 1H), 8.23-7.96 (m, 2H), 7.87 (d, J = 8.5 Hz, 1H), 7.80-7.51 (m, 3H), 7.24- 7.12 (m, 2H), 5.79- 5.44 (m, 4H), 4.97-4.68 (m, 1H), 4.62-4.46 (m, 2H), 4.45-3.96 (m, 4H), 3.19-3.02 (m, 1H), 2.20- 2.14 (m, 1H), 2.11- 1.87 (m, 7H), 1.86-1.76 (m, 3H), 1.65-1.50 (m, 3H), 1.12 (s, 18H) |
| 151 | ((((2- (((3S,6S,10aS)-3- (rel-trans)-3- cyano-4- phenylpyrrolidine-1- carbonyl)-6- methyl-5- oxodecahydro- pyrrolo[1,2-a] azocin-6-yl) carbamoyl) benzo[b]thiophen- 5-yl) difluoromethyl) phosphoryl)bis (oxy))bis (methylene) bis(2,2- dimethylpropanoate) | 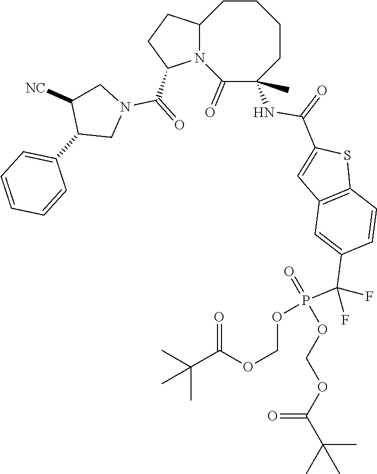 [SMILES: CC(C)(C)C(=O)OCOP(=O)(OCOC(=O)C(C)(C)C)C(F)(F)c1ccc2sc(C(=O)N[C@@]3(C)CCCCC4CC[C@@H](C(=O)N5C[C@H](c6ccccc6)[C@@H](C#N)C5)N4C3=O)cc2c1] | 913.3 [M + H]⁺ | ¹H NMR (400 MHz, DMSO-d₆) δ 8.35-7.99 (m, 4H), 7.68-7.25 (m, 6H), 5.90-5.32 (m, 4H), 4.69-4.50 (m, 2H), 4.33- 3.69 (m, 6H), 2.88- 2.72 (m, 1H), 2.34-1.61 (m, 12H), 1.54-1.41 (m, 2H), 1.20-1.06 m, 18H) |
| 152 | S,S′-(((((2- (((3S,6S,10aS)-3- ((3S,4R or 3R,4S)- 3-cyano-4- phenylpyrrolidine- 1-carbonyl)-5- oxodecahydro- pyrrolo[1,2-a] azocin-6- yl)carbamoyl) benzo[b]thiophen- 5-yl) difluoromethyl) phosphoryl)bis(oxy)) bis(ethane-2,1-diyl)) dibutanethioate | 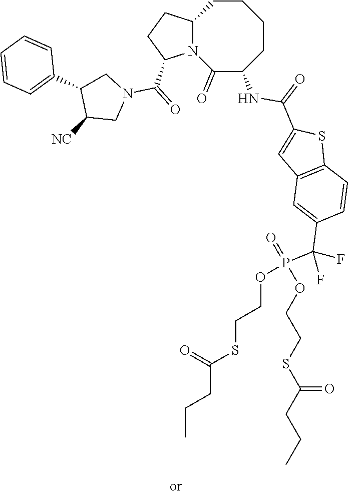 [SMILES: CCCC(=O)SCCOP(=O)(OCCSC(=O)CCC)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | | ¹H NMR (400 MHz, CDCl₃) δ 8.13-8.01 (m, 1H), 7.97-7.77 (m, 2H), 7.69-7.56 (m, 1H), 7.46- 7.27 (m, 5H), 5.18- 5.06 (m, 1H), 4.63-4.50 (m, 1H), 4.39-3.95 (m, 8H), 3.81-3.45 (m, 2H), 3.36-3.05 (m, 5H), 2.53 (t, J = 7.4 Hz, 4H), 2.31- 1.95 (m, 7H), 1.91- 1.80 (m, 2H), 1.78-1.60 (m, 7H), 0.94 (t, J = 7.4 Hz, 6H) |
| | | 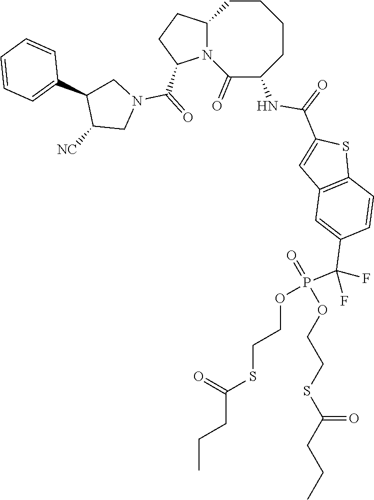 [SMILES: CCCC(=O)SCCOP(=O)(OCCSC(=O)CCC)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 153 | S,S′-(((((2- (((3S,6S,10aS)-3- ((3S,4R or 3R,4S)- 3-cyano-4- phenylpyrrolidine-1- carbonyl)-5- oxodecahydro- pyrrolo[1,2-a] azocin-6- yl)carbamoyl) benzo[b]thiophen- 5-yl) difluoromethyl) phosphoryl)bis (oxy))bis(butane- 4,1-diyl)) bis(3- methylbutane thioate) | 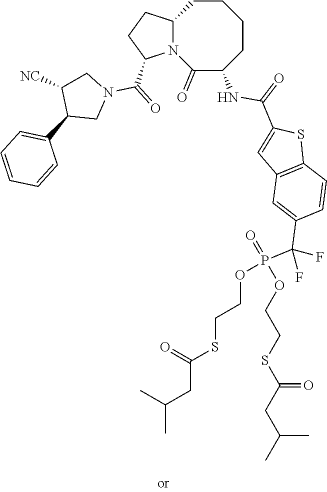 | 1015.2 [M + H]⁺ | ¹H NMR: (400 MHz, CDCl₃) δ 8.10-8.04 (m, 1H), 7.96-7.90 (m, 1H), 7.87 and 7.84 (s, 1H), 7.66-7.60 (m, 1H), 7.44- 7.21 (m, 6H), 5.18- 5.06 (m, 1H), 4.61-4.50 (m, 1H), 4.38-4.23 (m, 2H), 4.21-3.95 (m, 6H), 3.80-3.45 (m, 2H), 3.45- 3.12 (m, 1H), 2.85 (t, J = 7.1 Hz, 4H), 2.41 (d, J = 7.1 Hz, 4H), 2.28- 1.92 (m, 8H), 1.90- 1.56 (m, 14H), 0.94 (d, J = 6.7 Hz, 12H) |
| | | 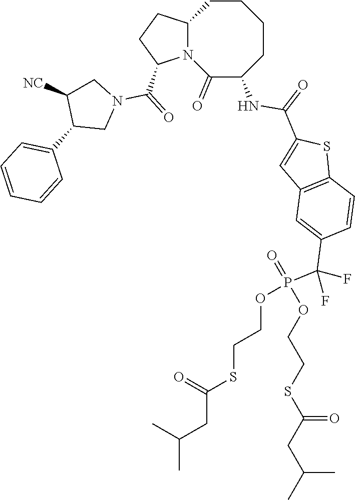 [SMILES: CC(C)CC(=O)SCCOP(=O)(OCCSC(=O)CC(C)C)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak was 2 used for | | |
| | | biological testing | | |
| 154 | S,S′-(((((2- (((3S,6S,10aS)-3- ((3S,4R or 3R,4S)- 3-cyano-4-(2- oxo-1,2- dihydropyridin-4- yl)pyrrolidine-1- carbonyl)-5- oxodecahydro- pyrrolo[1,2-a] azocin-6-yl) carbamoyl)benzo [b]thiophen-5- yl)difluoromethyl) phosphoryl)bis (oxy))bis(ethane- 2,1-diyl)) bis(3- methylbutane thioate) | 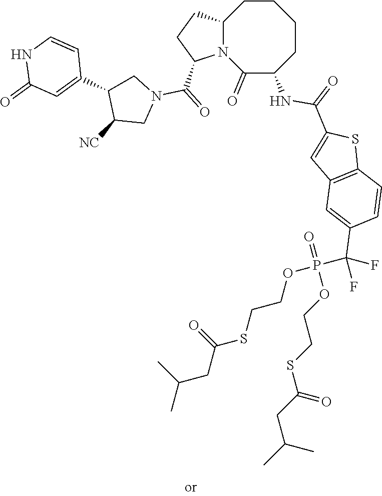 [SMILES: CC(C)CC(=O)SCCOP(=O)(OCCSC(=O)CC(C)C)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] | 976.3 [M + H]⁺ | ¹H NMR (400 MHz, CDCl₃) δ 8.17-7.83 (m, 1H), 7.69-7.54 (m, 1H), 7.44-7.30 (m, 1H), 6.80- 6.49 (m, 1H), 6.38- 6.17 (m, 1H), 5.18-5.05 (m, 1H), 4.63-4.46 (m, 1H), 4.44-4.31 (m, 1H), 4.31-4.06 (m, 6H), 4.04- 3.92 (m, 1H), 3.84- 3.60 (m, 2H), 3.56-3.04 (m, 6H), 2.48-2.37 (m, 4H), 2.30-2.03 (m, 9H), 2.02-1.87 (m, 3H), 1.85- 1.74 (m, 1H), 1.65 (dd, J = 12.0, 6.7 Hz, 1H), 1.03-0.87 (m, 12H) |
| | | 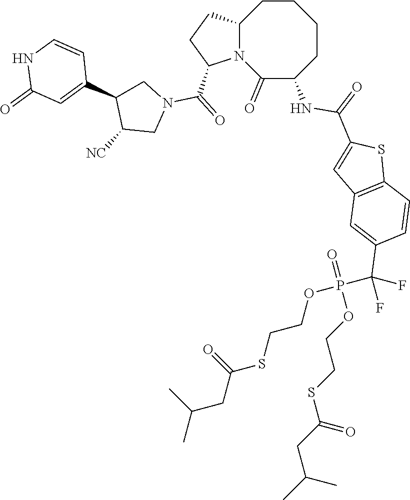 [SMILES: CC(C)CC(=O)SCCOP(=O)(OCCSC(=O)CC(C)C)C(F)(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] | | |
| | | SFC peak 2 was used for | | |
| | | biological testing | | |
| 155 | S,S′-(((((7- (((3S,6S,10aS)-3- ((3R,4S or 3S,4R)- 3-cyano-4-(2- oxo-1,2- dihydropyridin-4- yl)pyrrolidine-1- carbonyl)-5- oxodecahydro- pyrrolo[1,2-a] azocin-6- yl)carbamoyl) naphthalen-2- yl)difluoromethyl) phosphoryl)bis(oxy)) bis(ethane-2,1- diyl)) bis(3- methylbutane thioate) | 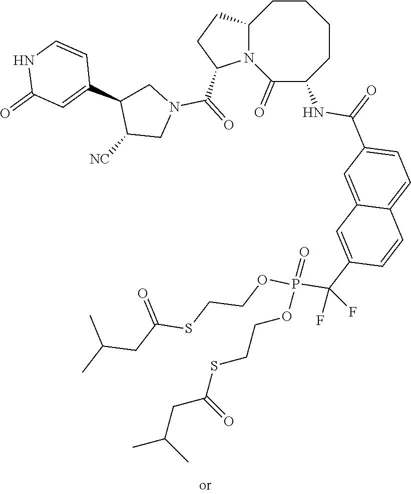 [SMILES: CC(C)CC(=O)SCCOP(=O)(OCCSC(=O)CC(C)C)C(F)(F)c1ccc2ccc(C(=O)N[C@H]3CCCC[C@H]4CC[C@@H](C(=O)N5C[C@H](c6cc[nH]c(=O)c6)[C@@H](C#N)C5)N4C3=O)cc2c1] | 970.3 [M + H]⁺ | ¹H NMR (400 MHz, CDCl₃) δ 8.68-8.39 (m, 5H), 8.38-8.16 (m, 1H), 8.09-7.68 (m, 5H), 7.44- 7.31 (m, 1H), 6.69- 6.46 (m, 1H), 6.28-6.10 (m, 1H), 5.27-5.04 (m, 1H), 4.64-4.44 (m, 2H), 4.40-4.05 (m, 6H), 3.99- 3.62 (m, 3H), 3.57- 3.24 (m, 2H), 3.21-3.05 (m, 5H), 2.46-2.37 (m, 4H), 2.28-2.00 (m, 9H), 2.00-1.85 (m, 3H), 1.83- 1.70 (m, 2H), 0.93 (d, J = 6.6 Hz, 12H) |
| | | 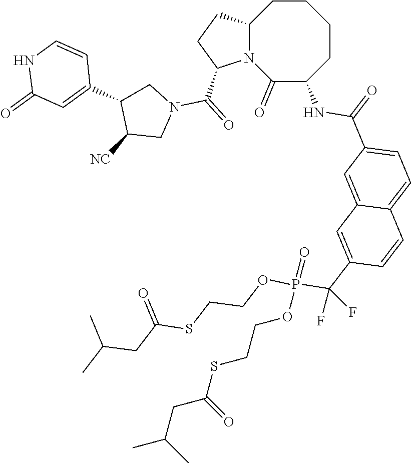 [SMILES: CC(C)CC(=O)SCCOP(=O)(OCCSC(=O)CC(C)C)C(F)(F)c1ccc2ccc(C(=O)N[C@H]3CCCC[C@H]4CC[C@@H](C(=O)N5C[C@@H](C#N)[C@H](c6cc[nH]c(=O)c6)C5)N4C3=O)cc2c1] | | |
| | | SFC peak 1 was used for | | |
| | | biological testing | | |
| 156 | S,S′-(((((2- (((3S,6S,10aS)-3- ((3S,4R or 3R,4S)- 3-cyano-4- phenylpyrrolidine-1- carbonyl)-5- oxodecahydro- pyrrolo[1,2-a] azocin-6- yl)carbamoyl) benzo[b]thiophen- 5-yl) difluoromethyl) phosphoryl)bis(oxy)) bis(ethane-2,1-diyl)) bis(2- methylpropane- thioate) | 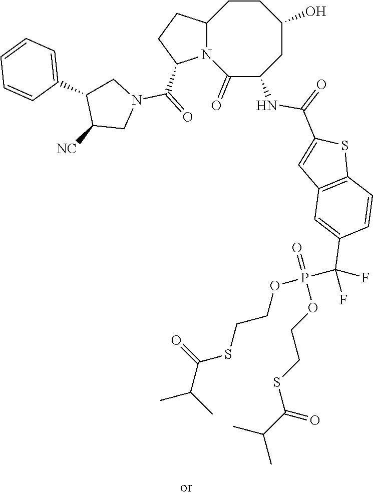 | 931.3 [M + H]⁺ | ¹H NMR (400 MHz, DMSO-d₆) δ 8.99-8.85 (m, 1H), 8.35 (d, J = 11.4 Hz, 1H), 8.25-8.07 (m, 2H), 7.67-7.26 (m, 6H), 5.07-4.83 (m, 1H), 4.57- 4.51 (m, 1H), 4.36- 3.53 (m, 11H), 3.14- 3.11 m, 4H), 2.77-2.67 (m, 2H), 2.28-1.51 (m, 12H), 1.18-0.95 (m, 12H) |
| | | 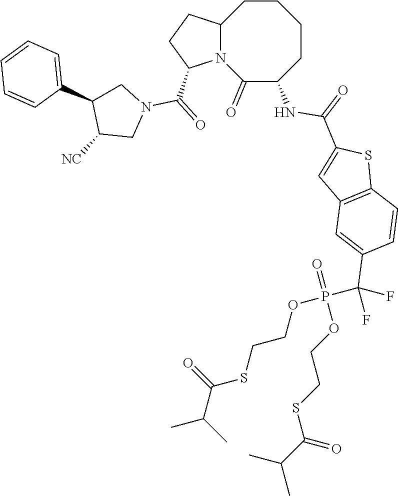 [SMILES: CC(C)C(=O)SCCOP(=O)(OCCSC(=O)C(C)C)C(F)(F)c1ccc2sc(C(=O)N[C@H]3CCCCC4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 157 | S,S′-(((((2- (((3S,6S,10aS)-3- (rel-(trans)-3- cyano-4-(2-oxo- 1,2-dihydropyridin- 4-yl) pyrrolidine-1- carbonyl)-5- oxodecahydro- pyrrolo[1,2-a] azocin-6-yl) carbamoyl)benzo [b]thiophen-5- yl)difluoromethyl) phosphoryl)bis(oxy)) bis(ethane-2,1- diyl)) bis(2,2- dimethylpropane- thioate) | 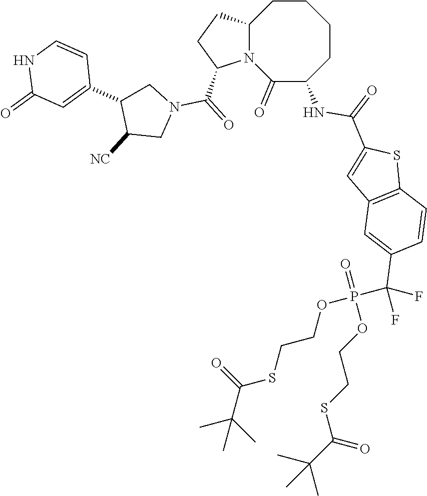 [SMILES: CC(C)(C)C(=O)SCCOP(=O)(OCCSC(=O)C(C)(C)C)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] | 976.3 [M + H]⁺ | ¹H NMR (400 MHz, CDCl₃) δ 8.18-7.81 (m, 4H), 7.79-7.55 (m, 1H), 7.49-7.29 (m, 1H), 6.76- 6.40 (m, 1H), 6.35- 6.14 (m, 1H), 5.22-5.03 (m, 1H), 4.67-4.28 (m, 3H), 4.28-3.84 (m, 6H), 3.84-3.25 (m, 3H), 3.23- 2.98 (m, 1H), 2.34- 1.84 (m, 9H), 1.83-1.61 (m, 3H), 1.22 (s, 18H) |
| 158 | diisopropyl (((((2- (((3S,6S,9aS)-5- oxo-3-(3-(pyridin- 3-yl)azetidine-1- carbonyl) octahydro-1H- pyrrolo[1,2-a] azepin-6-yl) carbamoyl)benzo [b]thiophen-5- yl)methyl) phosphoryl)bis (oxy))bis(methyl- ene))bis(carbonate) | 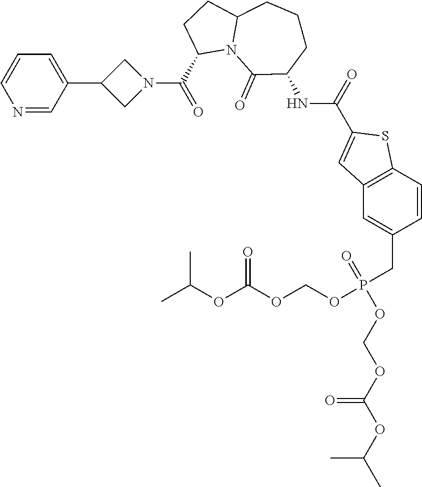 [SMILES: CC(C)OC(=O)OCOP(=O)(Cc1ccc2sc(C(=O)N[C@H]3CCCC4CC[C@@H](C(=O)N5CC(c6cccnc6)C5)N4C3=O)cc2c1)OCOC(=O)OC(C)C] | 814.9 [M + H]⁺ | ¹H NMR (400 MHz, CDCl₃) δ 8.83-8.45 (m, 2H), 7.99-7.34 (m, 7H), 5.64-5.59 (m, 4H), 5.05- 3.92 (m, 10H), 3.53- 3.30 (m, 2H), 2.41-2.07 (m, 6H), 1.98-1.69 (m, 4H), 1.30 (d, J = 5.3 Hz, 12H) |
| 159 | S,S′-(((((2- (((3S,6S,10aS)-3- ((3S,4R or 3R,4S)- 3-cyano-4- phenylpyrrolidine-1- carbonyl)-5- oxodecahydro- pyrrolo[1,2-a] azocin-6- yl)carbamoyl) benzo[b]thiophen-5- yl)difluoromethyl) phosphoryl)bis(oxy)) bis(ethane-2,1- diyl)) bis(3,3,3- trifluoro-2,2- dimethylpropane- thioate) | 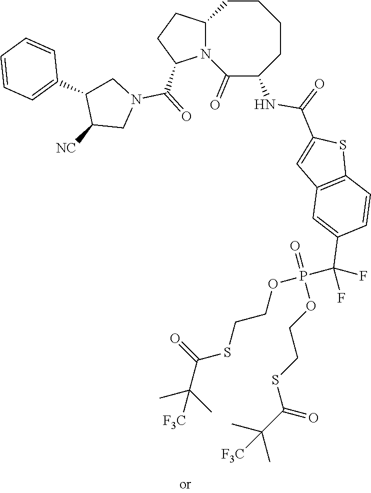 [SMILES: CC(C)(C(=O)SCCOP(=O)(OCCSC(=O)C(C)(C)C(F)(F)F)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1)C(F)(F)F] | 1067.3 [M + H]⁺ | ¹H NMR (400 MHz, CDCl₃) δ 8.07 (m, 1H), 7.97-7.81 (m, 2H), 7.64- 7.60 (m, 1H), 7.42- 7.30 (m, 6H), 5.20-5.10 (m, 1H), 4.58-4.55 (m, 1H), 4.31 4.25 (m, 2H), 4.26-4.18 (m, 3H), 4.16- 3.96 (m, 3H), 3.79- 3.47 (m, 2H), 3.12-3.14 (m, 4H), 2.31-2.06 (m, 5H), 1.92-1.62 (m, 8H), 1.46 (s, 12H), 1.26 (m, 2H) |
| | | 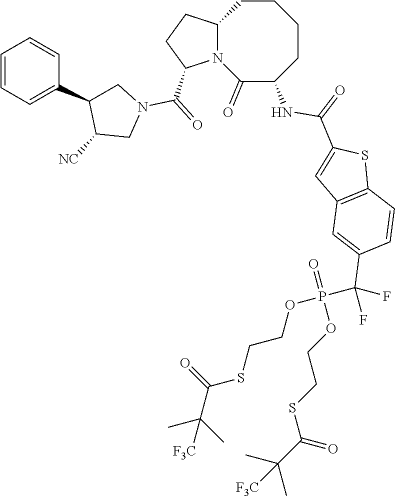 [SMILES: CC(C)(C(=O)SCCOP(=O)(OCCSC(=O)C(C)(C)C(F)(F)F)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1)C(F)(F)F] | | |
| | | SFC peak was 2 used for | | |
| | | biological testing | | |
| 160 | S,S′- ((((difluoro(7- (((3S,6S,10aS)-5- oxo-3-(3-(2-oxo-1,2- dihydropyridin-4- yl)pyrrolidine-1- carbonyl) decahydropyrrolo [1,2-a]azocin-6- yl)carbamoyl) naphthalen-2- yl)methyl) phosphoryl)bis(oxy)) bis(ethane-2,1-diyl)) bis(3-methylbutane thioate) | 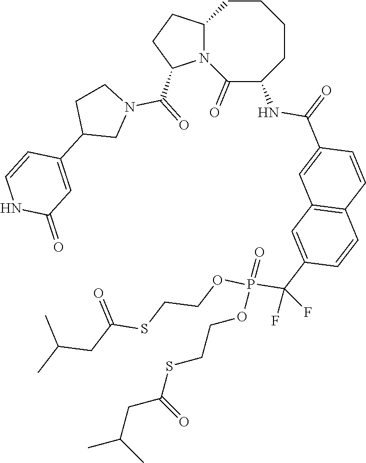 [SMILES: CC(C)CC(=O)SCCOP(=O)(OCCSC(=O)CC(C)C)C(F)(F)c1ccc2ccc(C(=O)N[C@H]3CCCC[C@H]4CC[C@@H](C(=O)N5CCC(c6cc[nH]c(=O)c6)C5)N4C3=O)cc2c1] | 945.4 [M + H]⁺ | ¹H NMR (400 MHz, CDCl₃) δ 8.48-8.42 (m, 1H), 8.22-8.19 (m, 1H), 8.01-7.91 (m, 3H), 7.76- 7.74 (m, 1H), 7.76- 7.41 (m, 2H), 6.89-6.49 (m, 2H), 5.20-5.17 (m, 1H), 4.61 (t, J = 7.9 Hz, 1H), 4.39-4.35 (m, 1H), 4.27-4.14 (m, 5H), 3.86- 3.67 (m, 2H), 3.58- 3.36 (m, 2H), 3.20-3.08 (m, 4H), 2.46-2.20 (m, 5H), 2.16-2.01 (m, 10H), 1.99-1.66 (m, 5H), 0.93 (d, J = 6.6, 1.3 Hz, 12H) |
| 161 | S,S′- ((((difluoro(2- (((3S,6S,10aR)-10- methyl-5-oxo-3-(3- (pyridin-3-yl) azetidine-1- carbonyl) decahydropyrrolo [1,2-a]azocin-6- yl)carbamoyl) benzo[b]thiophen- 5-yl)methyl) phosphoryl)bis(oxy)) bis(ethane-2,1-diyl)) bis(3- methylbutane thioate) | 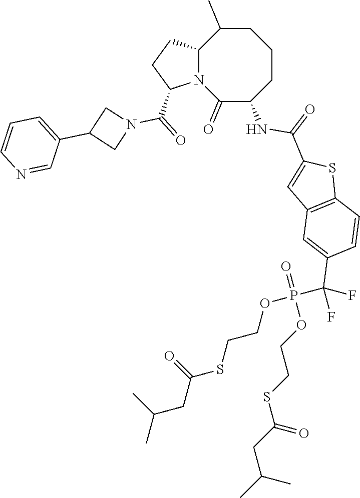 [SMILES: CC(C)CC(=O)SCCOP(=O)(OCCSC(=O)CC(C)C)C(F)(F)c1ccc2sc(C(=O)N[C@H]3CCCC(C)[C@H]4CC[C@@H](C(=O)N5CC(c6cccnc6)C5)N4C3=O)cc2c1] | 934.9 [M + H]⁺ | ¹H NMR (400 MHz, CDCl₃) δ 9.00-8.84 (m, 1H), 8.69-8.52 (m, 1H), 8.24-8.09 (m, 2H), 8.08- 7.84 (m, 2H), 7.83- 7.60 (m, 2H), 7.60-7.37 (m, 1H), 5.09-4.73 (m, 1H), 4.72-4.33 (m, 3H), 4.29-4.01 (m, 6H), 4.01- 3.79 (m, 1H), 3.24- 3.05 (m, 4H), 2.46-2.40 (m, 4H), 2.32-2.20 (m, 3H), 2.20-1.95 (m, 7H), 1.95-1.76 (m, 2H), 1.73- 1.66 (m, 2H), 0.97- 0.93 (m, 12H), 0.93- 0.89 (m, 3H) |
| 162 | S,S′-(((((2- (((3S,6S,10aR)-3- ((3R,4S or 3S,4R)-3- cyano-4-(2-oxo-1,2- dihydropyridin-4- yl)pyrrolidine-1- carbonyl)-10- methyl-5-oxodeca- hydropyrrolo[1,2-a] azocin-6-yl) carbamoyl)benzo [b]thiophen-5- yl)difluoromethyl) phosphoryl) bis(oxy))bis(ethane- 2,1-diyl)) bis(3- methylbutane thioate) | 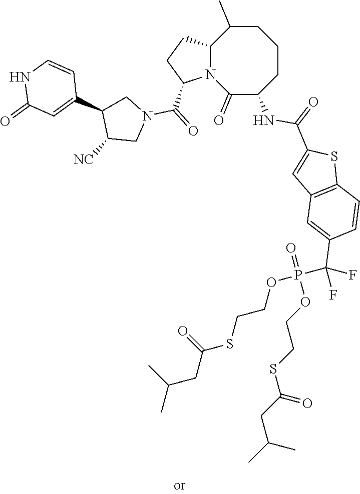 [SMILES: CC(C)CC(=O)SCCOP(=O)(OCCSC(=O)CC(C)C)C(F)(F)c1ccc2sc(C(=O)N[C@H]3CCCC(C)[C@H]4CC[C@@H](C(=O)N5C[C@H](c6cc[nH]c(=O)c6)[C@@H](C#N)C5)N4C3=O)cc2c1] | 990.3 [M + H]⁺ | ¹H NMR (400 MHz, CDCl₃) δ 8.30-8.03 (m, 2H), 7.97-7.76 (m, 2H), 7.68-7.56 (m, 1H), 7.42- 7.31 (m, 1H), 6.96- 6.52 (m, 1H), 6.32-6.19 (m, 1H), 4.75-4.57 (m, 1H), 4.56-4.42 (m, 1H), 4.41-4.03 (m, 8H), 4.01- 3.68 (m, 2H), 3.66- 3.25 (m, 2H), 3.22-3.05 (m, 5H), 2.46-2.38 (m, 4H), 2.34-2.24 (m, 1H), 2.23-2.04 (m, 8H), 1.97- 1.87 (m, 1H), 1.86- 1.78 (m, 1H), 1.77-1.71 (m, 1H), 1.69-1.61 (m, 1H), 0.97-0.88 (m, 15H) |
| | | 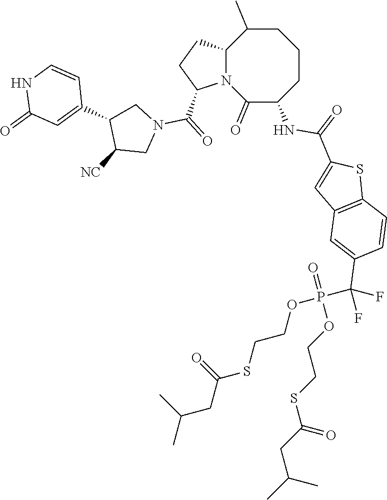 [SMILES: CC(C)CC(=O)SCCOP(=O)(OCCSC(=O)CC(C)C)C(F)(F)c1ccc2sc(C(=O)N[C@H]3CCCC(C)[C@H]4CC[C@@H](C(=O)N5C[C@@H](C#N)[C@H](c6cc[nH]c(=O)c6)C5)N4C3=O)cc2c1] | | |
| | | SFC peak 2 was used for | | |
| | | biological testing | | |
| 163 | S,S′-(((((2- (((3S,6S,10aS)-3- ((3R,4S or 3S,4R)-3- cyano-4- phenylpyrrolidine-1- carbonyl)-5- oxodecahydro- pyrrolo[1,2-a] azocin-6-yl) carbamoyl)benzo[b] thiophen-5-yl) difluoromethyl) phosphoryl)bis(oxy)) bis(ethane-2,1-diyl)) bis(3- methylbutane thioate) | 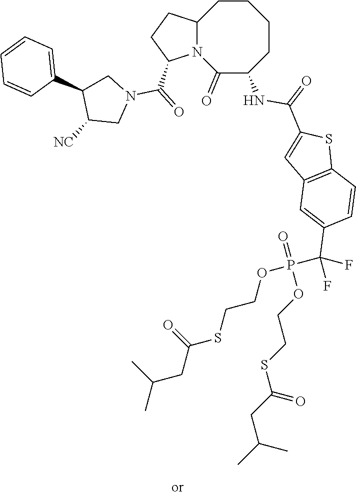 [SMILES: CC(C)CC(=O)SCCOP(=O)(OCCSC(=O)CC(C)C)C(F)(F)c1ccc2sc(C(=O)N[C@H]3CCCCC4CC[C@@H](C(=O)N5C[C@H](c6ccccc6)[C@@H](C#N)C5)N4C3=O)cc2c1] | 958.8 [M + H]⁺ | ¹H NMR (400 MHz, CDCl₃) δ 8.16-8.01 (m, 1H), 7.97-7.89 (m, 1H), 7.88-7.79 (m, 1H), 7.66- 7.59 (m, 1H), 7.44- 7.30 (m, 5H), 5.20-5.05 (m, 1H), 4.64-4.49 (m, 1H), 4.39-3.97 (m, 8H), 3.81-3.49 (m, 2H), 3.31- 3.05 (m, 5H), 2.43 (d, J = 7.1 Hz, 4H), 2.30- 2.10 (m, 6H), 2.09-1.76 (m, 6H), 1.72-1.61 (m, 2H), 0.94 (d, J = 6.7 Hz, 12H) |
| | | 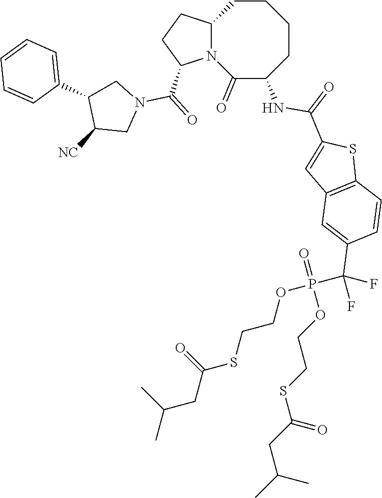 [SMILES: CC(C)CC(=O)SCCOP(=O)(OCCSC(=O)CC(C)C)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | | |
| | | SFC peak 2 was used for | | |
| | | biological testing | | |
| 164 | ((((S)-(2-(((3S,6S, 10aS)-3-((3R,4S or 3S,4R)-3-cyano-4- (2-oxo-1,2-dihydro- pyridin-4-yl)pyrrolidine- 1-carbonyl)-5-oxodeca- hydropyrrolo[1,2-a] azocin-6-yl) carbamoyl)benzo[b] thiophen-5-yl)fluoro- methyl)phosphoryl)bis (oxy))bis(methylene) diisopropyl bis(carbonate) or ((((R)-(2-(((3S,6S,10aS)- 3-((3R,4S or 3S,4R)-3- cyano-4-(2-oxo-1,2- dihydropyridin-4- yl)pyrrolidine-1- carbonyl)-5-oxodeca- hydropyrrolo[1,2-a] azocin-6-yl) carbamoyl)benzo[b] thiophen-5-yl) fluoromethyl) phosphoryl)bis(oxy)) bis(methylene) diisopropyl bis(carbonate) | 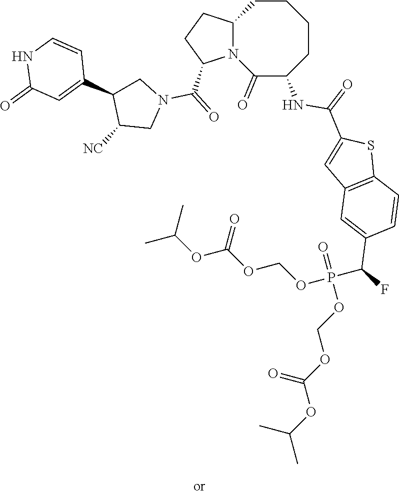 [SMILES: CC(C)OC(=O)OCOP(=O)(OCOC(=O)OC(C)C)[C@H](F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] | 902.3 [M + H]⁺ | ¹H NMR (400 MHz, CDCl₃) δ 7.99-7.92 (m, 1H), 7.91-7.79 (m, 2H), 7.64-7.29 (m, 3H), 7.03- 6.73 (m, 1H), 6.70- 6.53 (m, 1H), 5.90 (dd, J = 44.1, 6.9 Hz, 1H), 5.71- 5.59 (m, 4H), 5.21- 5.03 (m, 1H), 4.97-4.83 (m, 2H), 4.60-4.45 (m, 1H), 4.39-3.93 (m, 4H), 3.79-3.12 (m, 3H), 2.29- 1.94 (m, 6H), 1.93- 1.55 (m, 6H), 1.36-1.23 (m, 12H) |
| | | 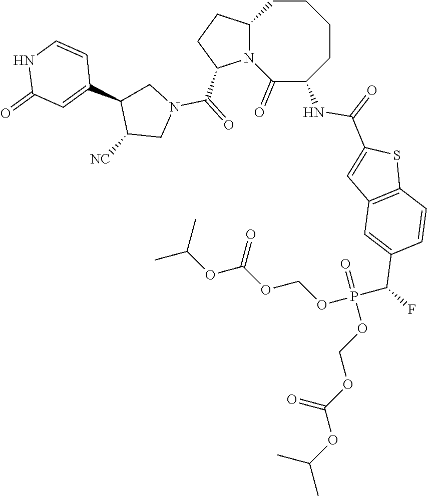 [SMILES: CC(C)OC(=O)OCOP(=O)(OCOC(=O)OC(C)C)[C@@H](F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] | | |
| | | 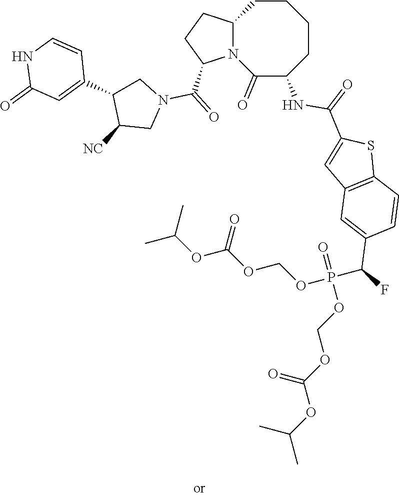 [SMILES: CC(C)OC(=O)OCOP(=O)(OCOC(=O)OC(C)C)[C@H](F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] | | |
| | | 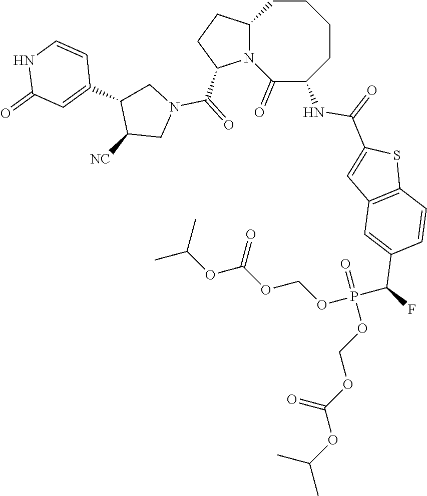 [SMILES: CC(C)OC(=O)OCOP(=O)(OCOC(=O)OC(C)C)[C@H](F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] | | |
| | | (SFC peak 1 was used for | | |
| | | biological testing, Peak 2 of | | |
| | | phosphonate building block | | |
| | | was used for synthesis) | | |
| 165 | S-(2-((((2- (((3S,6S,10aS)-3- ((3S,4R or 3R,4S)-3- cyano-4- phenylpyrrolidine-1- carbonyl)-5- oxodecahydro- pyrrolo[1,2-a] azocin-6- yl)carbamoyl) benzo[b]thiophen- 5-yl)difluoromethyl) (2-(pivaloylthio) ethoxy)phosphoryl) oxy)ethyl) butanethioate | 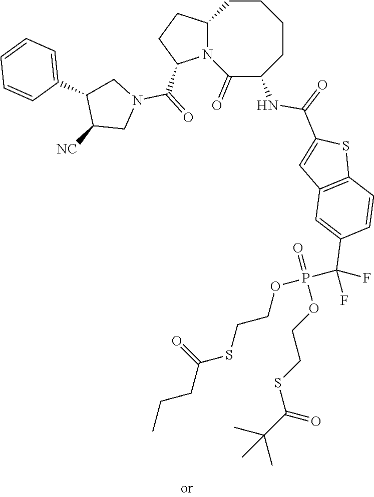 [SMILES: CCCC(=O)SCCOP(=O)(OCCSC(=O)C(C)(C)C)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 967.4 [M + H]⁺ | ¹H NMR (400 MHz, DMSO-d₆) δ 8.96-8.88 (m, 1H), 8.38-8.31 (m, 1H), 8.24-8.18 (m, 1H), 8.17-8.12 (m, 1H), 7.62- 7.55 (m, 1H), 7.47- 7.26 (m, 5H), 5.02-4.89 (m, 1H), 4.54 (q, J = 9.0 Hz, 1H), 4.36-3.90 (m, 7H), 3.86-3.77 (m, 1H), 3.70-3.47 (m, 2H), 3.34- 3.24 (m, 1H), 3.17- 3.06 (m, 4H), 2.57-2.50 (m, 2H), 2.33-2.22 (m, 1H), 2.13-1.76 (m, 8H), 1.69-1.47 (m, 5H), 1.16- 1.13 (m, 9H), 0.88- 0.81 (m, 3H) |
| | | 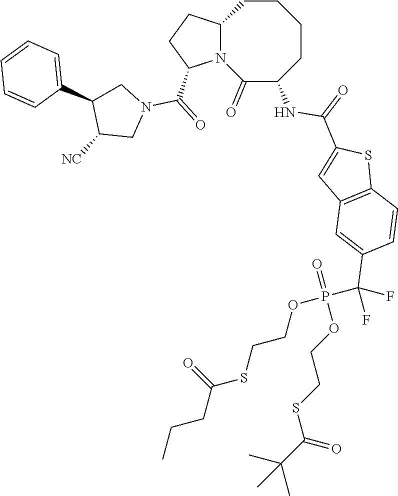 [SMILES: CCCC(=O)SCCOP(=O)(OCCSC(=O)C(C)(C)C)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 166 | S-(2-((((2- (((3S,6S,10aS)-3- ((3R,4S or 3S,4R)-3- cyano-4- phenylpyrrolidine-1- carbonyl)-5- oxodecahydropyrrolo [1,2-a]azocin-6- yl)carbamoyl)benzo[b] thiophen-5- yl)difluoromethyl) (((isopropoxycarbonyl) oxy)methoxy) phosphoryl)oxy)ethyl)- 3-methylbutane thioate | 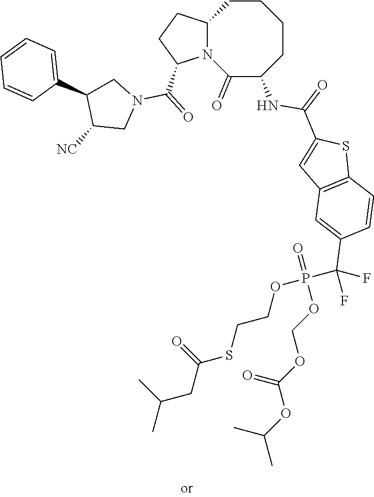 [SMILES: CC(C)CC(=O)SCCOP(=O)(OCOC(=O)OC(C)C)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | 931.3 [M + H]⁺ | ¹H NMR (400 MHz, CDCl₃) δ 8.13-8.06 (m, 1H), 7.97-7.90 (m, 1H), 7.89-7.81 (m, 1H), 7.67- 7.61 (m, 1H), 7.44- 7.27 (m, 5H), 7.26-7.20 (m, 1H), 5.73-5.59 (m, 2H), 5.19-5.06 (m, 1H), 4.98-4.86 (m, 1H), 4.61- 4.50 (m, 1H), 4.40- 3.03 (m, 12H), 2.44- 2.39 (m, 2H), 2.30-1.70 (m, 11H), 1.32 (d, J = 6.0 Hz, 6H), 0.94 (d, J = 6.0 Hz, 6H) |
| | | 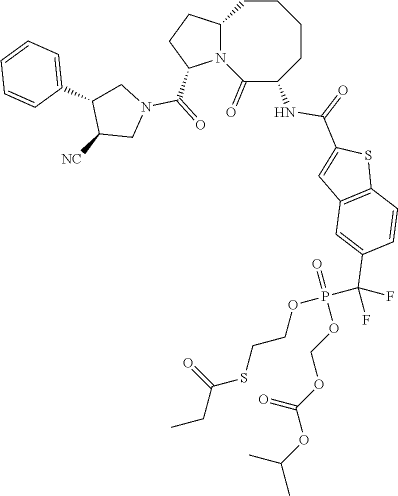 [SMILES: CCC(=O)SCCOP(=O)(OCOC(=O)OC(C)C)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | | |
| | | SFC peak 2 was used for | | |
| | | biological testing | | |
| 167 | S,S′-(((((7- (((1R,3S,6S,11aS)-3- ((3S,4R or 3R,4S)-3- cyano-4-(2-oxo-1,2- dihydropyridin-4- yl)pyrrolidine-1- carbonyl)-1- hydroxy-5- oxodecahydropyrrolo [1,2-a]azocin-6- yl)carbamoyl) naphthalen-2- yl)difluoromethyl) phosphoryl)bis(oxy)) bis(ethane-2,1-diyl)) bis(3-methylbutane thioate) | 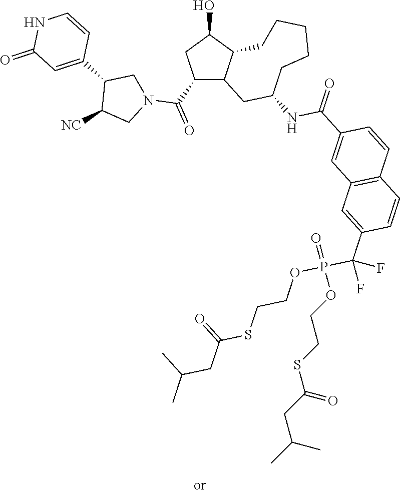 | 999.9 [M + H]⁺ | ¹H (400 MHz, CDCl₃) δ 8.44-8.40 (m, 1H), 8.28- 8.05 (m, 2H), 7.98- 7.61 (m, 4H), 7.16 (m, 1H), 6.50-6.42 (m, 1H), 6.12-5.95 (m, 1H), 5.36-5.10 (m, 1H), 4.97- 4.73 (m, 1H), 4.32- 3.97 (m, 8H), 4.02-3.90 (m, 1H), 3.60-3.19 (m, 2H), 3.17-3.11 (m, 4H), 3.09-3.01 (m, 1H), 2.57- 2.43 (m, 1H), 2.43- 2.40 (m, 4H), 2.26-2.20 (m, 1H), 2.16-2.11 (m, 5H), 1.92-1.62 (m, 8H), 0.94-0.91 (m, 12H) |
| | | 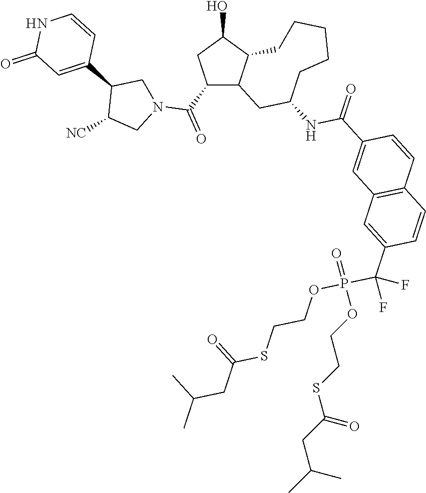 [SMILES: CC(C)CC(=O)SCCOP(=O)(OCCSC(=O)CC(C)C)C(F)(F)c1ccc2ccc(C(=O)N[C@H]3CCCCC[C@@H]4C(C3)[C@H](C(=O)N3C[C@H](c5cc[nH]c(=O)c5)[C@@H](C#N)C3)C[C@H]4O)cc2c1] | | |
| | | SFC peak 2 was used for | | |
| | | biological testing | | |
| 168 | S,S′-(((((7- (((3S,6S,10aS)-3- ((3S,4R or 3R,4S)-3- cyano-4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphoryl)bis(oxy)) bis(ethane-2,1-diyl)) bis(3-methylbutane thioate) | 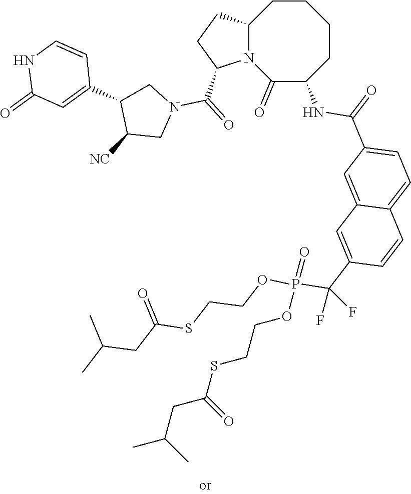 [SMILES: CC(C)CC(=O)SCCOP(=O)(OCCSC(=O)CC(C)C)C(F)(F)c1ccc2ccc(C(=O)N[C@H]3CCCC[C@H]4CC[C@@H](C(=O)N5C[C@@H](C#N)[C@H](c6cc[nH]c(=O)c6)C5)N4C3=O)cc2c1] | 970.4 [M + H]⁺ | ¹H NMR (400 MHz, CDCl₃) δ 8.55-8.41 (m, 2H), 8.35-8.15 (m, 1H), 8.09-7.68 (m, 5H), 7.47- 7.27 (m, 2H), 6.79- 6.43 (m, 1H), 6.37-6.11 (m, 1H), 5.27-5.04 (m, 1H), 4.61-4.47 (m, 1H), 4.44-4.12 (m, 7H), 4.11- 3.89 (m, 1H), 3.79- 3.42 (m, 2H), 3.30-3.07 (m, 5H), 2.49-2.35 (m, 4H), 2.30-2.05 (m, 7H), 2.05-1.65 (m, 9H), 1.04- 0.84 (m, 12H) |
| | | 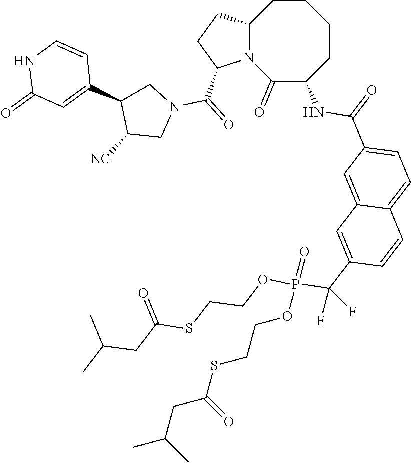 [SMILES: CC(C)CC(=O)SCCOP(=O)(OCCSC(=O)CC(C)C)C(F)(F)c1ccc2ccc(C(=O)N[C@H]3CCCC[C@H]4CC[C@@H](C(=O)N5C[C@H](c6cc[nH]c(=O)c6)[C@@H](C#N)C5)N4C3=O)cc2c1] | | |
| | | SFC peak 2 was used for | | |
| | | biological testing | | |
| 169 | ((((2- (((4S,7S,11aS)-4-((rel- trans)-3-cyano-4- phenylpyrrolidine-1- carbonyl)-6- oxodecahydro-2H- pyrido[1,2-a]azocin-7- yl)carbamoyl)benzo[b] thiophen-5-yl) difluoromethyl) phosphroyl)bis(oxy)) bis(methylene) bis(2,2- dimethylpropaneoate) | 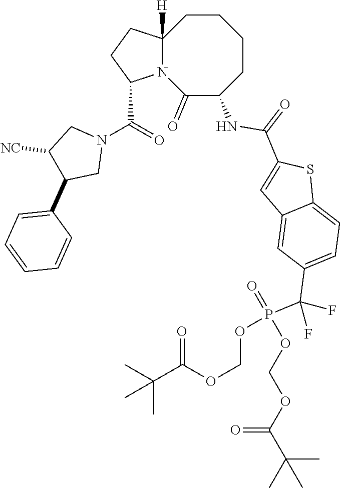 [SMILES: CC(C)(C)C(=O)OCOP(=O)(OCOC(=O)C(C)(C)C)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 911.0 [M − H]⁻ | ¹H NMR (600 MHz, DMSO-d₆) δ 8.09 (d, J = 15.9 Hz, 1H), 7.93 (t, J = 7.1 Hz, 1H), 7.88-7.78 (m, 1H), 7.62 (d, J = 8.2 Hz, 1H), 7.53-7.29 (m, 6H), 5.73 (dd, J = 12.6, 5.0 Hz, 2H), 5.69-5.60 (m, 2H), 5.10 (s, 1H), 4.91 (dd, J = 7.7, 4.2 Hz, 1H), 4.37-4.21 (m, 2H), 4.15-3.78 (m, 3H), 3.72- 3.61 (m, 1H), 3.27- 3.13 (m, 1H), 2.22 (s, 1H), 1.93 (s, 3H), 1.83 (s, 4H), 1.69 (d, J = 37.4 Hz, 2H), 1.51 (q, J = 16.3, 12.6 Hz, 3H), 1.41 (d, J = 6.9 Hz, 1H), 1.20 (s, 18H) |
| 170 | ((((2- (((4S,7S,10aS)-4-(rel- (trans)-3-cyano-4- phenylpyrrolidine-1- carbonyl)-6- oxodecahydropyrido [1,2-a]azepin-7- yl)carbamoyl)benzo[b] thiophen-5- yl)difluoromethyl) phosphoryl)bis(oxy)) bis(methylene) bis(2,2- dimethylpropanoate) | 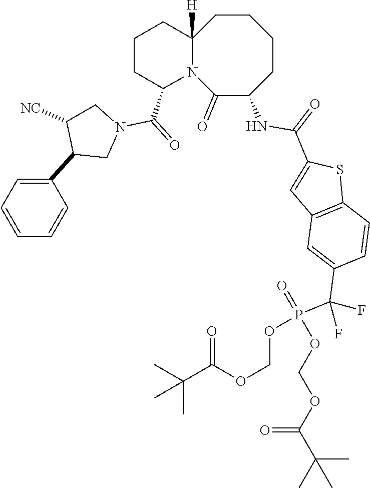 [SMILES: CC(C)(C)C(=O)OCOP(=O)(OCOC(=O)C(C)(C)C)C(F)(F)c1ccc2sc(C(=O)N[C@H]3CCCC[C@H]4CCC[C@@H](C(=O)N5C[C@@H](C#N)[C@H](c6ccccc6)C5)N4C3=O)cc2c1] | | ¹H NMR (600 MHz, DMSO-d₆) δ 8.09 (s, 1H), 7.94 (d, J = 8.5 Hz, 1H), 7.90-7.83 (m, 1H), 7.71 (app m, 1H), 7.63 (d, J = 8.6 Hz, 1H), 7.50- 7.30 (m, 5H), 5.78- 5.69 (m, 2H), 5.65 (m, 2H), 5.07-4.96 (m, 2H), 4.58-4.07 (m, 1H), 4.07- 3.99 (m, 2H), 3.90- 3.62 (m, 2H), 3.58-3.17 (m, 1H), 2.61-2.39 (m, 1H), 2.28-2.07 (m, 1H), 1.92 (dt, J = 34.5, 17.1 Hz, 5H), 1.78 (s, 3H), 1.70 (s, 1H), 1.20 (s, 18H) |
| 171 | ((((2- (((4S,7S,11aS)-4-(3- (1H-imidazol-1- yl)azetidine-1-carbonyl)- 6-oxodecahydro-2H- pyrido[1,2-a]azocin-7- yl)carbamoyl)benzo[b] thiophen-5- yl)difluoromethyl) phosphoryl)bis(oxy)) bis(methylene) bis(2,2- dimethylpropanoate) | 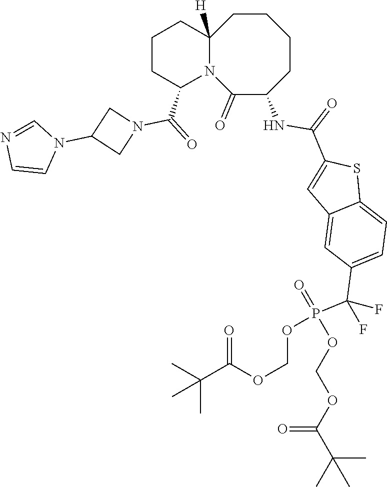 [SMILES: CC(C)(C)C(=O)OCOP(=O)(OCOC(=O)C(C)(C)C)C(F)(F)c1ccc2sc(C(=O)N[C@H]3CCCC[C@H]4CCC[C@@H](C(=O)N5CC(n6ccnc6)C5)N4C3=O)cc2c1] | 864.4 [M + H]⁺ | ¹H NMR (600 MHz, DMSO-d₆) δ 9.87 (s, 1H), 8.17-7.49 (m, 7H), 6.05 (s, 2H), 5.74-5.48 (m, 3H), 4.98 (s, 1H), 4.63 (s, 2H), 4.39 (s, 1H), 4.24 (s, 1H), 4.06 (s, 1H), 2.41 (s, 1H), 2.18 (s, 1H), 1.84 (s, 5H), 1.45 (s, 4H), 1.34-0.88 (m, 18H) |
| 172 | (((difluoro(2- (((4S,7S,11aS)-4-(3- (morpholine-4- carbonyl)azetidine-1- carbonyl)-6- oxodecahydro-2H- pyrido[1,2- a]azocin-7-yl) carbamoyl)benzo[b] thiophen-5- yl)methyl)phosphoryl) bis(oxy))bis(methylene) bis(2,2- dimethylpropanoate) | 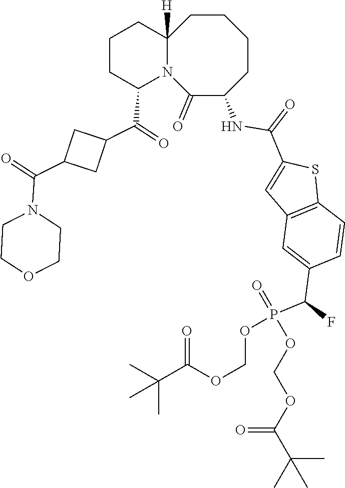 | 911.2 [M + H]⁺ | ¹H NMR (600 MHz, DMSO-d₆) δ 8.05 (app. m, 1H), 7.99-7.86 (m, 1H), 7.86-7.76 (m, 1H), 7.63 (d, J = 8.6 Hz, 1H), 7.54 (m, 1H), 5.72 (m, 2H), 5.69-5.59 (m, 2H), 5.18-5.06 (m, 1H), 4.78 (m, 1H), 4.68 (m, 1H), 4.60-4.41 (m, 1H), 4.27 (m, 2H), 4.16-4.04 (m, 1H), 3.75-3.60 (m, 7H), 3.57 (s, 1H), 1.99-1.77 (m, 6H), 1.77-1.59 (m, 3H), 1.59-1.28 (m, 5H), 1.23-1.18 (s, 18H) |
| 173 | ((((2- (((4S,7S,10aS)-4-(3- (1H-imidazol-1-yl) azetidine-1-carbonyl)- 6-oxodecahydropyrido [1,2-a]azepin-7-yl) carbamoyl)benzo[b] thiophen-5-yl) difluoromethyl) phosphoryl)bis(oxy)) bis(methylene) bis(2,2- dimethylpropanoate) | 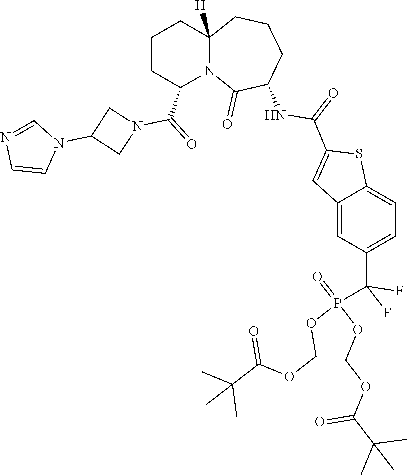 [SMILES: CC(C)(C)C(=O)OCOP(=O)(OCOC(=O)C(C)(C)C)C(F)(F)c1ccc2sc(C(=O)N[C@H]3CCC[C@H]4CCC[C@@H](C(=O)N5CC(n6ccnc6)C5)N4C3=O)cc2c1] | 850.2 [M + H]⁺ | |
| 174 | (((difluoro(2- (((3S,6S,10aS)-3-(3- fluoro-3-phenylazetidine- 1-carbonyl)-5- oxodecahydropyrrolo [1,2-a]azocin-6-yl) carbamoyl)benzo[b] thiophen-5-yl)methyl) phosphoryl)bis(oxy)) bis(methylene) bis(2,2- dimethylpropanoate) | 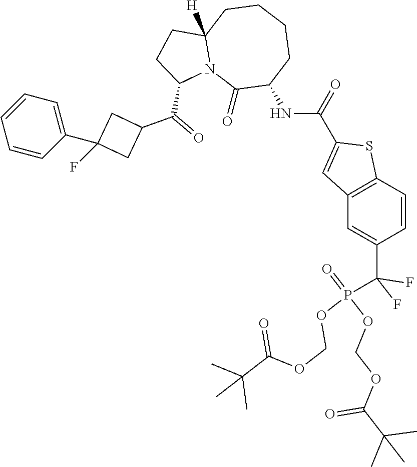 [SMILES: CC(C)(C)C(=O)OCOP(=O)(OCOC(=O)C(C)(C)C)C(F)(F)c1ccc2sc(C(=O)N[C@H]3CCCC[C@H]4CC[C@@H](C(=O)C5CC(F)(c6ccccc6)C5)N4C3=O)cc2c1] | 878.2 [M + H]⁺ | ¹H NMR (600 MHz, DMSO-d₆) δ 8.99 (dd, J = 17.9, 7.1 Hz, 1H), 8.32 (d, J = 28.3 Hz, 1H), 8.20 (t, J = 8.1 Hz, 1H), 8.09 (d, J = 11.2 Hz, 1H), 8.02 (d, J = 8.7 Hz, 1H), 7.46 (m, 6H), 5.81-5.64 (m, 4H), 4.98-4.86 (m, 2H), 4.67 (m, 1H), 4.41-4.31 (m, 2H), 4.27 (m, 2H), 2.20 (m, 1H), 2.09-2.00 (m, 1H), 1.90-1.74 (m, 6H), 1.64 (m, 1H), 1.59 (m, 1H), 1.50 (s, 1H), 1.10 (s, 18H) |
| 175 | S,S′- ((((difluoro(2- (((3S,6S,9aS)-5-oxo-3- (3-(pyridin-3- yl)azetidine-1- carbonyl)octahydro- 1H-pyrrolo[1,2-a] azepin-6-yl) carbamoyl)benzo[b] thiophen-5-yl)methyl) phosphoryl)bis(oxy)) bis(ethane-2,1-diyl)) bis(2,2- dimethylpropanethioate) | 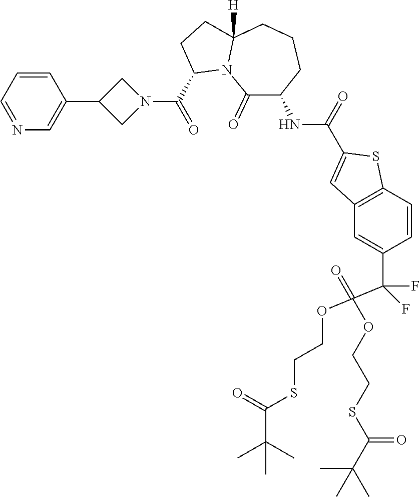 | | ¹H NMR (600 MHz, DMSO-d₆) δ 9.14 (s, 1H), 8.64 (d, J = 20.8 Hz, 1H), 8.40 (s, 1H), 8.29- 8.07 (m, 2H), 8.03-7.83 (m, 2H), 7.74-7.61 (m, 2H), 4.88 (m, 1H), 4.70 (m, 1H), 4.51 (m, 2H), 4.25-4.19 (m, 2H), 4.15 (m, 2H), 3.94 (m, 2H), 3.13 (m, 4H), 2.41-2.14 (m, 4H), 2.04 (m, 4H), 1.91 (m, 4H), 1.23 (s, 18H) |
| 176 | S-(2-((((2- (((3S,6S,9aS)-3-(3-(3- cyanophenyl) azetidine-1-carbonyl)-5- oxooctahydro-1H- pyrrolo[1,2-a]azepin-6- yl)carbamoyl)benzo[b] thiophen-5-yl) difluoromethyl)(((3- methylbutanoyl)thio) methoxy)phosphoryl) oxy)ethyl) 3- methylbutane thioate | 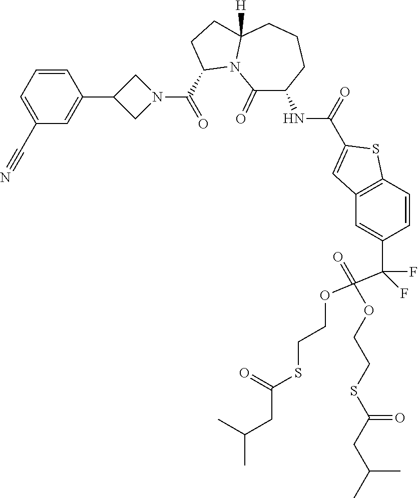 | 931.2 [M + H]⁺ | ¹H NMR (400 MHz, methanol-d₄) δ 8.28- 8.18 (m, 2H), 8.15-8.09 (m, 1H), 7.97 (s, 1H), 7.79-7.73 (m, 1H), 7.70 (d, J = 8.0 Hz, 1H), 7.68- 7.62 (m, 1H), 7.60- 7.49 (m, 1H), 4.93 (d, J = 1.6 Hz, 1H), 4.78 (d, J = 10.4 Hz, 1H), 4.70 (t, J = 8.0 Hz, 1H), 4.63-4.54 (m, 2H), 4.41 (t, J = 8.0 Hz, 1H), 4.26-4.16 (m, 4H), 4.11-4.07 (m, 1H), 4.05-3.94 (m, 1H), 3.17- 3.07 (m, 4H), 2.81 (s, 1H), 2.22-2.15 (m, 4H), 2.10-1.96 (m, 7H), 1.94- 1.80 (m, 4H), 1.24 (t, J = 7.2 Hz, 1H), 0.93 (d, J = 4.8 Hz, 12H) |
| 177 | S,S′- ((((difluoro(2- (((3S,6S,9aS)-5-oxo- 3-(3-(pyridin-3- yl)azetidine-1- carbonyl)octahydro-1H- pyrrolo[1,2-a]azepin-6- yl)carbamoyl)benzo[b] thiophen-5- yl)methyl)phosphoryl) bis(oxy))bis(ethane-2,1- diyl)) bis(3- methylbutane thioate) | 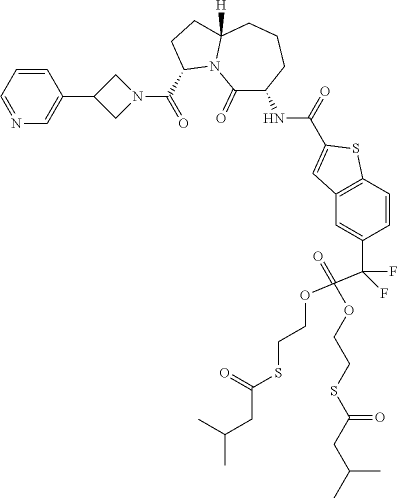 | 907.3 [M + H]⁺ | ¹H NMR (400 MHz, CDCl₃) δ 8.67-8.55 (m, 2H), 8.15 (d, J = 11.6 Hz, 1H), 7.98 (d, J = 8.8 Hz, 1H), 7.92-7.87 (m, 1H), 7.74-7.68 (m, 2H), 7.42- 7.32 (m, 1H), 5.10 (m, 1H), 4.74-4.58 (m, 3H), 4.57-4.39 (m, 1H), 4.29- 4.18 (m, 5H), 4.12- 4.05 (m, 1H), 4.01-3.88 (m, 2H), 3.19-2.96 (m, 4H), 2.35-2.21 (m, 2H), 2.21-2.17 (m, 4H), 2.13- 2.06 (m, 5H), 2.03 (s, 1H), 1.96 (t, J = 10.0 Hz, 2H), 1.86-1.70 (m, 2H), 0.95 (d, J = 6.4 Hz, 12H) |
| 178 | S-(2- (((difluoro(2- (((3S,6S,9aS)-3-(3-((S- methylsulfonimidoyl) methyl)azetidine-1- carbonyl)-5- oxooctahydro-1H- pyrrolo[1,2-a]azepin-6- yl)carbamoyl)benzo[b] thiophen-5-yl)methyl) (((3-methylbutanoyl) thio)methoxy) phosphoryl)oxy)ethyl) 3-methylbutane thioate | 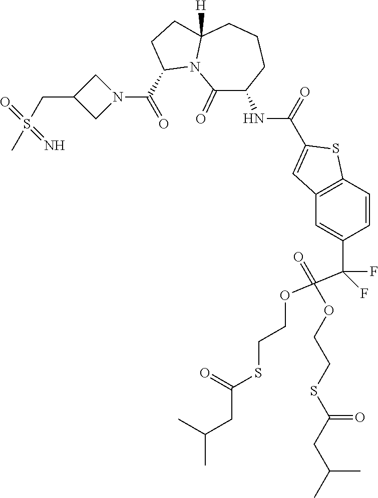 | 921 1015.3 [M + H]⁺ | ¹H NMR (400 MHz, CDCl₃) δ 8.14 (d, J = 6.8 Hz, 1H), 7.97 (d, J = 8.4 Hz, 1H), 7.89 (d, J = 10.8 Hz, 1H), 7.70 (d, J = 8.8 Hz, 1H), 4.71-4.62 (m, 1H), 4.52-4.44 (m, 2H), 4.27-4.16 (m, 5H), 3.97- 3.88 (m, 2H), 3.61- 3.44 (m, 2H), 3.39-3.27 (m, 1H), 3.07-3.02 (m, 4H), 2.29-2.21 (m, 2H), 2.21-2.18 (m, 4H), 2.13- 2.04 (m, 3H), 2.04- 1.98 (m, 3H), 1.97-1.89 (m, 2H), 1.84-1.77 (m, 2H), 1.26 (s, 3H), 0.95- (d, J = 6.8 Hz, 12H), 0.91- 0.82 (m, 2H) |
| 179 | S,S′- ((((difluoro(2- (((3S,6S,9aS)-5-oxo-3- (3-(pyridin-3-yl) azetidine-1- carbonyl)octahydro-1H- pyrrolo[1,2-a]azepin-6- yl)carbamoyl)benzo[b] thiophen-5- yl)methyl)phosphoryl) bis(oxy))bis(ethane- 2,1-diyl)) dibutanethioate | 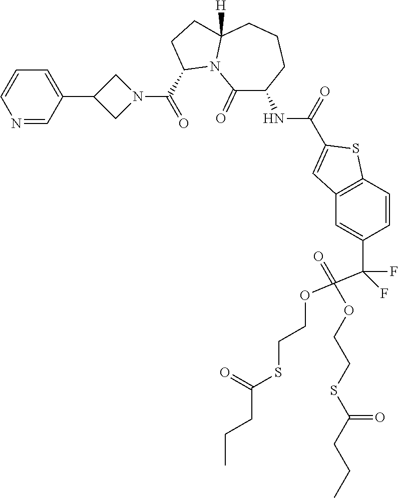 | 879.3 [M + H]⁺ | ¹H NMR (400 MHz, CDCl₃) δ 8.68-8.54 (m, 2H), 8.15 (d, J = 11.6 Hz, 1H), 7.98 (d, J = 8.4 Hz, 1H), 7.92-7.66 (m, 4H), 7.44-7.31 (m, 1H), 5.07- 4.38 (m, 4H), 4.30- 4.03 (m, 6H), 4.02-3.87 (m, 2H), 3.19-2.98 (m, 4H), 2.29 (t, J = 7.2 Hz, 4H), 2.25-2.19 (m, 1H), 2.06 (d, J = 3.2 Hz, 4H), 1.96 (t, J = 10.0 Hz, 2H), 1.86-1.80 (m, 1H), 1.74 (d, J = 11.6 Hz, 1H), 1.67- 1.63 (m, 3H), 1.32 (m, 2H), 0.95 (t, J = 7.2 Hz, 6H) |
| 180 | (((difluoro(2- (((3S,6S,9aS)-5-oxo-3- (3-(pyridin-3-yl) azetidine-1- carbonyl)octahydro-1H- pyrrolo[1,2-a]azepin-6- yl)carbamoyl)benzo[b] thiophen-5-yl)methyl) phosphoryl)bis(oxy)) bis(methylene) diisopropyl bis(carbonate) | 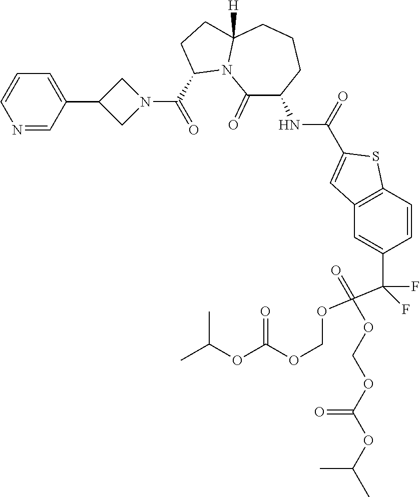 | 851.4 [M + H]⁺ | ¹H NMR (400 MHz, CDCl₃) δ 8.68-8.54 (m, 2H), 8.12 (d, J = 9.2 Hz, 1H), 7.95 (m, 1H), 7.92- 7.85 (m, 1H), 7.75-7.70 (m, 1H), 7.65 (d, J = 8.8 Hz, 1H), 7.47-7.33 (m, 1H), 5.78-5.62 (m, 4H), 4.98-4.84 (m, 2H), 4.75- 4.50 (m, 4H), 4.26- 4.18 (m, 1H), 4.12-4.04 (m, 1H), 4.03-3.87 (m, 2H), 2.34-2.18 (m, 2H), 1.78-1.55 (m, 8H), 1.32 (d, J = 6.0 Hz, 12H) |
| 181 | S,S′- ((((difluoro(2- (((3S,6S,10aS)-5-oxo- 3-(3-(pyridin-3-yl) azetidine-1- carbonyl)decahydro- pyrrolo[1,2-a]azocin-6- yl)carbamoyl)benzo[b] thiophen-5- yl)methyl)phosphoryl) bis(oxy))bis(ethane-2,1- diyl)) bis(2,2- dimethylpropanethioate) | 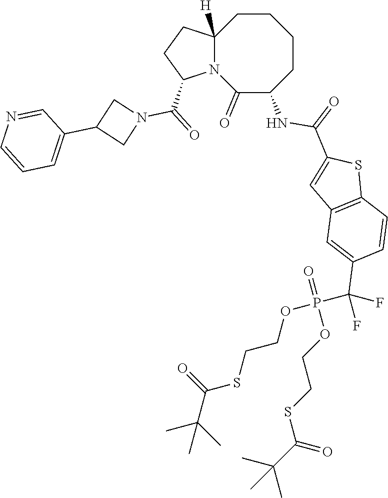 [SMILES: CC(C)(C)C(=O)SCCOP(=O)(OCCSC(=O)C(C)(C)C)C(F)(F)c1ccc2sc(C(=O)N[C@H]3CCCC[C@H]4CC[C@@H](C(=O)N5CC(c6cccnc6)C5)N4C3=O)cc2c1] | 921.1 [M + H]⁺ | ¹H NMR (400 MHz, acetonitrile-d₃) δ 8.30- 8.12 (m, 2H), 7.82 (d, J = 9.2 Hz, 1H), 7.76-7.62 (m, 3H), 7.55-7.48 (m, 1H), 7.32 (app. t, J = 7.6 Hz, 1H), 7.09-6.95 (m, 1H), 4.79-4.71 (m, 1H), 4.35-4.20 (m, 2H), 4.16- 4.07 (m, 2H), 4.01 (d, J = 8.8 Hz, 1H), 3.91- 3.82 (m, 4H), 3.66-3.58 (m, 2H), 2.81 (t, J = 6.4 Hz, 4H), 1.85-1.76 (m, 4H), 1.53-1.45 (m, 3H), 1.45-1.24 (m, 5H), 0.88 (s, 18H) |
| 182 | S,S′- (((3S,6S,10aS)-3-(3- (1H-imidazol-4-yl) azetidine-1-carbonyl)- 5-oxodecahydropyrrolo [1,2-a]azocin-6- yl)carbamoyl)benzo[b] thiophen-5-yl) difluoromethyl) phosphoryl)bis(oxy)) bis(ethane-2,1-diyl)) bis(2,2- dimethylpropanethioate) | 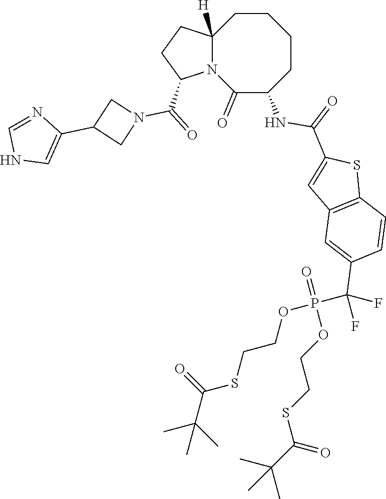 [SMILES: CC(C)(C)C(=O)SCCOP(=O)(OCCSC(=O)C(C)(C)C)C(F)(F)c1ccc2sc(C(=O)N[C@H]3CCCC[C@H]4CC[C@@H](C(=O)N5CC(c6c[nH]cn6)C5)N4C3=O)cc2c1] | 910.3 [M + H]⁺ | ¹H NMR (400 MHz, acetonitrile-d₃) δ 8.11 (s, 1H), 8.04-7.96 (m, 2H), 7.77 (d, J = 6.8 Hz, 1H), 7.61 (d, J = 8.4 Hz, 1H), 7.53 (s, 1H), 6.89 (d, J = 3.2 Hz, 1H), 5.07-4.97 (m, 1H), 4.46 (d, J = 7.6 Hz, 1H), 4.42-4.36 (m, 1H), 4.32-4.07 (m, 7H), 4.00-3.78 (m, 2H), 3.10 (t, J = 6.2 Hz, 4H), 2.23- 2.03 (m, 3H), 2.02- 1.96 (m, 2H), 1.92-1.86 (m, 1H), 1.80-1.72 (m, 2H), 1.71-1.64 (m, 1H), 1.64-1.54 (m, 2H), 1.18 (s, 18H) |

Representative Procedure for Amino Acid Coupling Conditions of Linker Acids Esters with Amino Acid Cores for Construction of Analogues

### Preparation of ((((R)- or (S)-(2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)fluoromethyl)phosphoryl)bis(oxy))bis(methylene)bis(2,2-dimethylpropanoate)) (183)

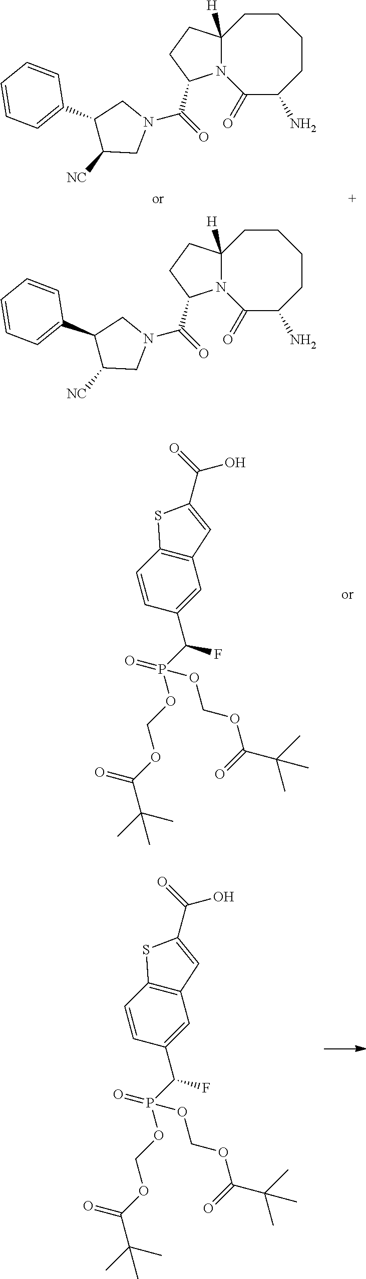

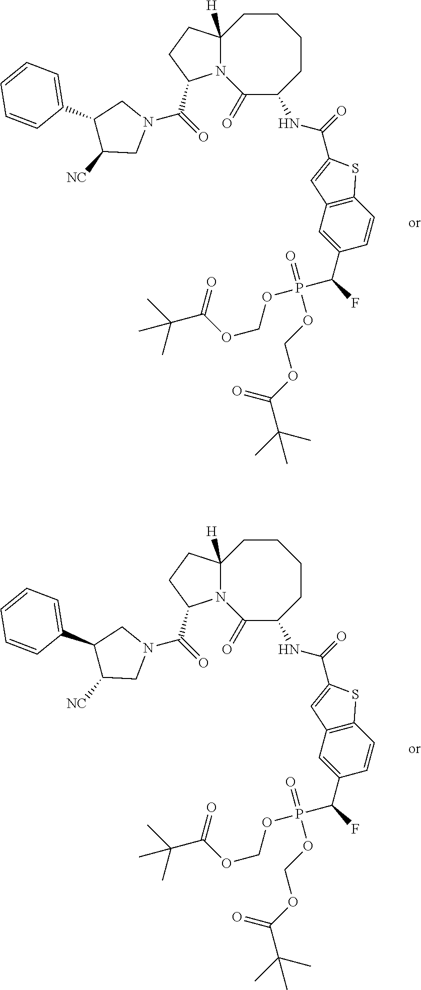 [SMILES: CC(C)(C)C(=O)OCOP(=O)(OCOC(=O)C(C)(C)C)[C@H](F)c1ccc2sc(C(=O)N[C@H]3CCCC[C@H]4CC[C@@H](C(=O)N5C[C@@H](C#N)[C@H](c6ccccc6)C5)N4C3=O)cc2c1 ; CC(C)(C)C(=O)OCOP(=O)(OCOC(=O)C(C)(C)C)[C@H](F)c1ccc2sc(C(=O)N[C@H]3CCCC[C@H]4CC[C@@H](C(=O)N5C[C@H](c6ccccc6)[C@@H](C#N)C5)N4C3=O)cc2c1]

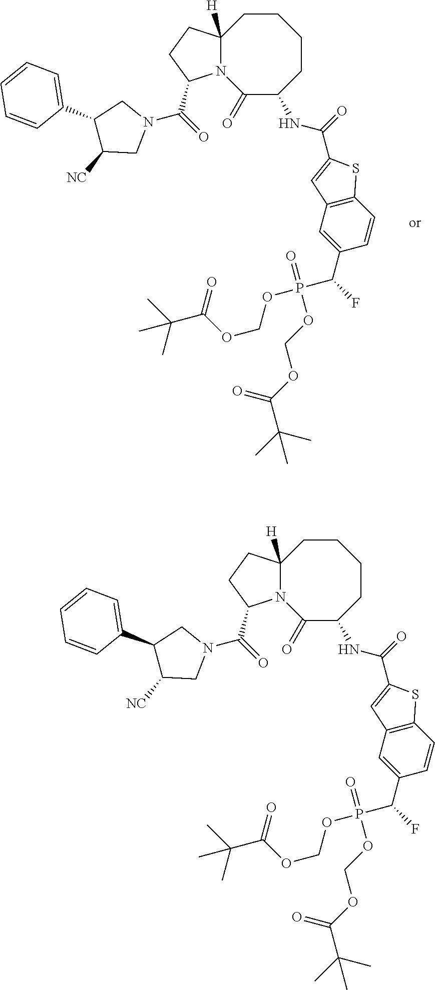 [SMILES: CC(C)(C)C(=O)OCOP(=O)(OCOC(=O)C(C)(C)C)[C@@H](F)c1ccc2sc(C(=O)N[C@H]3CCCC[C@H]4CC[C@@H](C(=O)N5C[C@@H](C#N)[C@H](c6ccccc6)C5)N4C3=O)cc2c1 ; CC(C)(C)C(=O)OCOP(=O)(OCOC(=O)C(C)(C)C)[C@@H](F)c1ccc2sc(C(=O)N[C@H]3CCCC[C@H]4CC[C@@H](C(=O)N5C[C@H](c6ccccc6)[C@@H](C#N)C5)N4C3=O)cc2c1]

To a solution of (3S,4R or 3R,4S)-1-[(3S,6S,10aS)-6-amino-5-oxo-decahydropyrrolo[1,2-a]azocine-3-carbonyl]-4-phenylpyrrolidine-3-carbonitrile (pyrrolidine peak 2) (45.6 mg, 120 μmol, 1.2 eq), 5-[(S)-[bis({[(2,2-dimethylpropanoyl)oxy]methoxy})phosphoryl](fluoro)methyl]-1-benzothiophene-2-carboxylic acid (phosphonate peak 2) (51.8 mg, 0.10 mmol, eq) and N,N-diisopropylethylamine (104 μL, 600 μmol, 6 eq) in DMF (3 mL) was added HATU (45.6 mg, 120 μmol, 1.2 eq). The reaction mixture was stirred at room temperature for 15 min and the solution was subsequently purified by reverse phase chromatography using a C18 column (gradient 5% to 100% acetonitrile in water) to yield ({[(R)- or (S)-(2-{[(3S,6S,10aS)-3-[(3S,4R or 3R,4S)-3-cyano-4-phenylpyrrolidine-1-carbonyl]-5-oxo-decahydropyrrolo[1,2-a]azocin-6-yl]carbamoyl}-1-benzothiophen-5-yl)(fluoro)methyl]({[(2,2-dimethylpropanoyl)oxy]methoxy})phosphoryl}oxy)methyl 2,2-dimethylpropanoate (182) (36.4 mg, 0.04136 mmol, 41.3%) as a white solid. LCMS (ESI) m/z=881.2 [M+H]⁺; ¹H NMR (400 MHz, acetonitrile-d₃) δ 8.03-7.91 (m, 3H), 7.56-7.46 (m, 2H), 7.46-7.29 (m, 5H), 5.99 (dd, J=43.0, 7.8 Hz, 1H), 5.67-5.52 (m, 4H), 5.10-4.96 (m, 1H), 4.63-4.53 (m, 1H), 4.42-4.23 (m, 2H), 4.13-3.95 (m, 1H), 3.85-3.68 (m, 1H), 3.65-3.31 (m, 3H), 2.35-2.21 (m, 1H), 2.12-1.96 (m, 5H), 1.91-1.66 (m, 4H), 1.65-1.54 (m, 2H), 1.12 (s, 18H).

The following compounds in Table 37 were prepared according to the representative procedure described above for the synthesis of ({[(R)- or (S)-(2-{[(3S,6S,10aS)-3-[(3S,4R or 3R,4S)-3-cyano-4-phenylpyrrolidine-1-carbonyl]-5-oxo-decahydropyrrolo[1,2-a]azocin-6-yl]carbamoyl}-1-benzothiophen-5-yl)(fluoro)methyl]({[(2,2-dimethylpropanoyl)oxy]methoxy})phosphoryl}oxy)methy, 2,2-dimethylpropanoate (182) and using the appropriate starting materials and modifications.

TABLE 37

| Com- | | | | |
|---|---|---|---|---|
| pound | Name | Structure | LCMS | NMR |
| 184 | ((((S)- or (R)-(2- (((3S,6S, 10aS)- 3-((3S,4R or 3R,4S)-3- cyano-4- phenyl- pyrrolidine- 1- carbonyl)-5- oxodeca- hydropyrrolo [1,2- a]azocin-6- yl)carbamoyl) benzo[b]thio phen-5-yl) fluoromethyl) phosphoryl) bis(oxy)) bis(methylene) bis(2,2- dimethyl- propanoate) | 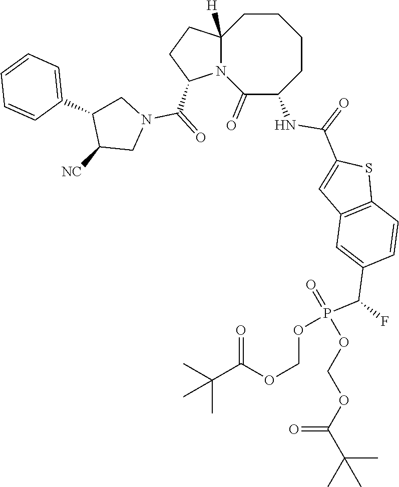 [SMILES: CC(C)(C)C(=O)OCOP(=O)(OCOC(=O)C(C)(C)C)[C@@H](F)c1ccc2sc(C(=O)N[C@H]3CCCC[C@H]4CC[C@@H](C(=O)N5C[C@@H](C#N)[C@H](c6ccccc6)C5)N4C3=O)cc2c1] or | 881.2 [M + H]⁺ | ¹H NMR (400 MHz, acetonitrile-d₃) δ 8.02- 7.95 (m, 2H), 7.94 (s, 1H), 7.59-7.47 (m, 2H), 7.45-7.29 (m, 5H), 5.99 (dd, J = 44.7, 8.8 Hz, 1H), 5.65-5.54 (m, 4H), 5.11- 4.93 (m, 1H), 4.63- 4.51 (m, 1H), 4.44-4.22 (m, 2H), 4.13-3.93 (m, 1H), 3.84-3.68 (m, 1H), 3.67-3.29 (m, 3H), 2.39- 2.20 (m, 1H), 2.13- 1.96 (m, 5H), 1.90-1.67 (m, 4H), 1.65-1.50 (m, 2H), 1.10 (s, 18H) |
| | | 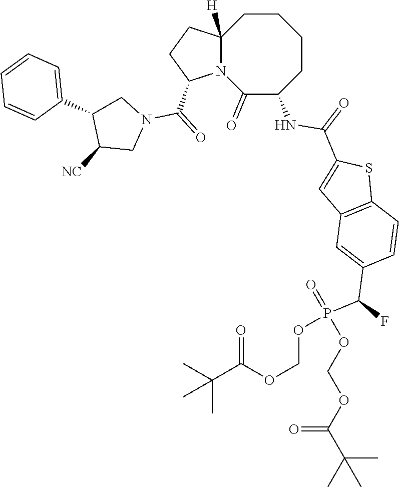 [SMILES: CC(C)(C)C(=O)OCOP(=O)(OCOC(=O)C(C)(C)C)[C@H](F)c1ccc2sc(C(=O)N[C@H]3CCCC[C@H]4CC[C@@H](C(=O)N5C[C@@H](C#N)[C@H](c6ccccc6)C5)N4C3=O)cc2c1] or | | |
| | | 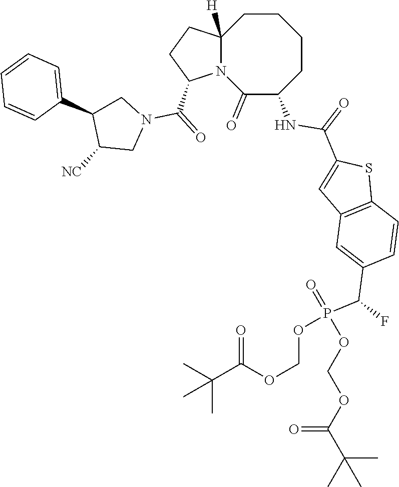 [SMILES: CC(C)(C)C(=O)OCOP(=O)(OCOC(=O)C(C)(C)C)[C@@H](F)c1ccc2sc(C(=O)N[C@H]3CCCC[C@H]4CC[C@@H](C(=O)N5C[C@H](c6ccccc6)[C@@H](C#N)C5)N4C3=O)cc2c1] or | | |
| | | 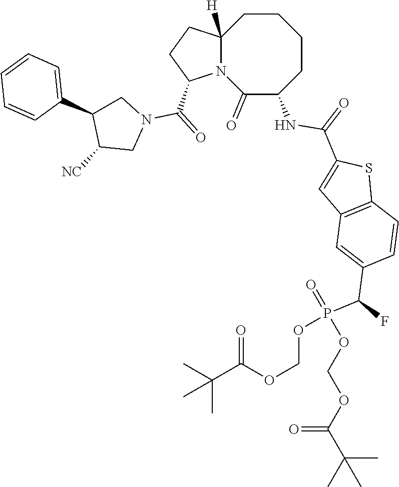 [SMILES: CC(C)(C)C(=O)OCOP(=O)(OCOC(=O)C(C)(C)C)[C@H](F)c1ccc2sc(C(=O)N[C@H]3CCCC[C@H]4CC[C@@H](C(=O)N5C[C@H](c6ccccc6)[C@@H](C#N)C5)N4C3=O)cc2c1] (SFC peak 2 was used for biological testing, Peak 1 of phosphonate building block was used for synthesis) | | |
| 185 | ((((2- (((3S,6S,8S, 10aR)-3- ((3R,4S or 3S,4R)-3- cyano-4- phenyl- pyrrolidine-1- carbonyl)-8- hydroxy-5- oxodeca- hydropyrrolo [1,2- a]azocin-6- yl)carbamoyl) benzo[b]thio phen-5- yl)difluoro- methyl)phos- phoryl)bis(oxy)) bis(methylene) bis(2,2- dimethyl- propanoate) | 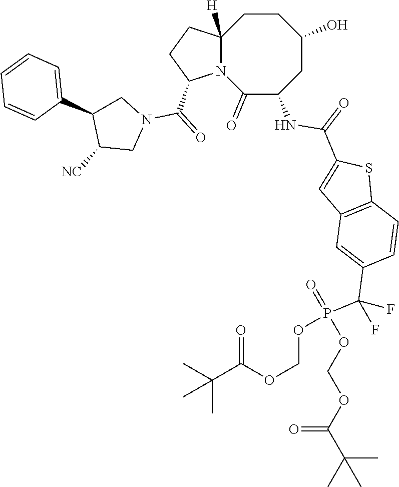 [SMILES: CC(C)(C)C(=O)OCOP(=O)(OCOC(=O)C(C)(C)C)C(F)(F)c1ccc2sc(C(=O)N[C@H]3C[C@@H](O)CC[C@H]4CC[C@@H](C(=O)N5C[C@H](c6ccccc6)[C@@H](C#N)C5)N4C3=O)cc2c1] or | 915.4 [M + H]⁺ | ¹H NMR (400 MHz, acetonitrile-d₃) δ 8.16- 7.94 (m, 3H), 7.65-7.55 (m, 2H), 7.48-7.28 (m, 5H), 5.72-5.58 (m, 4H), 5.09-4.97 (m, 1H), 4.58- 4.42 (m, 2H), 4.34- 4.22 (m, 1H), 4.15-3.87 (m, 3H), 3.81-3.33 (m, 4H), 2.40-2.29 (m, 2H), 2.27-2.15 (m, 2H), 2.11- 1.98 (m, 3H), 1.89- 1.75 (m, 2H), 1.74-1.63 (m, 1H), 1.16 (s, 18H) |
| | | 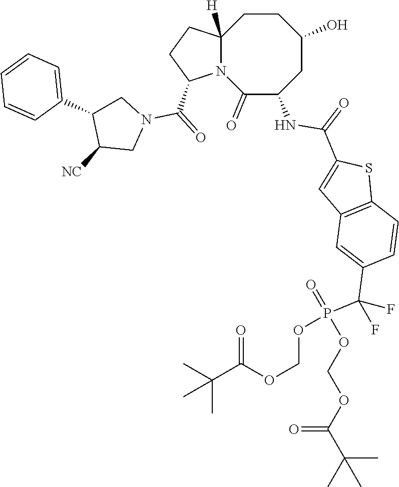 [SMILES: CC(C)(C)C(=O)OCOP(=O)(OCOC(=O)C(C)(C)C)C(F)(F)c1ccc2sc(C(=O)N[C@H]3C[C@@H](O)CC[C@H]4CC[C@@H](C(=O)N5C[C@@H](C#N)[C@H](c6ccccc6)C5)N4C3=O)cc2c1] SFC peak 1 was used for biological testing | | |
| 186 | (((difluoro(2- (((3S,6S, 10aS)-6- methyl- 3-(3- (morpholine- 4- carbonyl) azetidine-1- carbonyl)-5- oxodeca- hydropyrrolo [1,2-a] azocin-6- yl)carbamoyl) benzo[b] thiophen-5- yl)methyl) phosphoryl)bis (oxy))bis (methylene) bis(2,2- dimethyl- propanoate) | 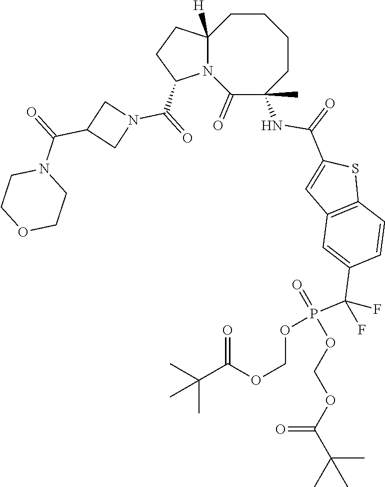 [SMILES: CC(C)(C)C(=O)OCOP(=O)(OCOC(=O)C(C)(C)C)C(F)(F)c1ccc2sc(C(=O)N[C@@]3(C)CCCC[C@H]4CC[C@@H](C(=O)N5CC(C(=O)N6CCOCC6)C5)N4C3=O)cc2c1] | 911.3 [M + H]⁺ | ¹H NMR (400 MHz, CDCl₃) δ 8.17-8.03 (m, 1H), 8.00-7.91 (m, 1H), 7.88-7.78 (m, 1H), 7.69- 7.59 (m, 1H), 5.77- 5.61 (m, 4H), 4.74-4.64 (m, 1H), 4.63-4.53 (m, 1H), 4.52-4.37 (m, 1H), 4.35-4.24 (m, 1H), 4.20- 4.03 (m, 1H), 3.92- 3.44 (m, 5H), 3.42-3.24 (m, 2H), 3.22-2.79 (m, 1H), 2.31-2.19 (m, 1H), 2.18-2.05 (m, 2H), 2.05- 1.94 (m, 2H), 1.88-1.85 (m, 1H), 1.72 (s, 3H), 1.70-1.58 (m, 5H), 1.55 (d, J = 10.8 Hz, 1H), 1.42- 1.29 (m, 2H), 1.20 (s, 18H) |
| 187 | (((difluoro(2- (((3S,6S,9aS)- 3-(3- (morpholine- 4- carbonyl) azetidine-1- carbonyl)-5- oxooctahydro- 1H- pyrrolo[1,2- a]azepin-6- yl)carbamoyl) benzo[b] thiophen-5- yl)methyl) phosphoryl)bis (oxy))bis (methylene) bis(2,2- dimethyl- propanoate) | 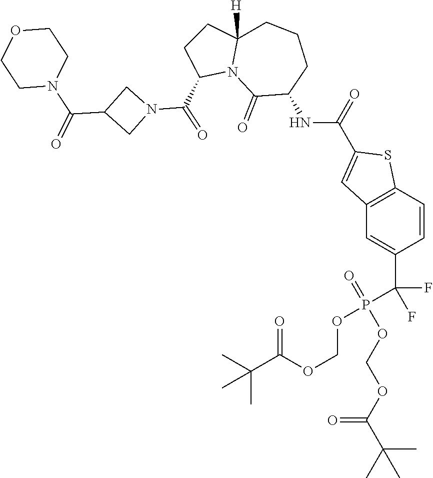 [SMILES: CC(C)(C)C(=O)OCOP(=O)(OCOC(=O)C(C)(C)C)C(F)(F)c1ccc2sc(C(=O)N[C@H]3CCC[C@H]4CC[C@@H](C(=O)N5CC(C(=O)N6CCOCC6)C5)N4C3=O)cc2c1] | 883.4 [M + H]⁺ | ¹H NMR (400 MHz, methanol-d₄) δ 8.19- 8.04 (m, 1H), 7.97-7.82 (m, 2H), 7.70-7.60 (m, 1H), 5.88-5.48 (m, 4H), 4.77-4.49 (m, 3H), 4.44- 4.25 (m, 1H), 4.24- 4.06 (m, 1H), 4.00-3.81 (m, 1H), 3.78-3.50 (m, 7H), 3.40-3.17 (m, 2H), 2.34 (s, 2H), 2.10-1.78 (m, 9H), 1.21 (s, 18H) |
| 188 | (((difluoro(2- (((3S,6S,9aS)- 5-oxo-3-(3- (pyridin-2- yl)azetidine- 1- carbonyl) octahydro-1H- pyrrolo[1,2- a]azepin-6- yl)carbamoyl) benzo[b] thiophen-5- yl)methyl) phosphoryl)bis (oxy))bis (methylene) bis(2,2- dimethyl- propanoate) | 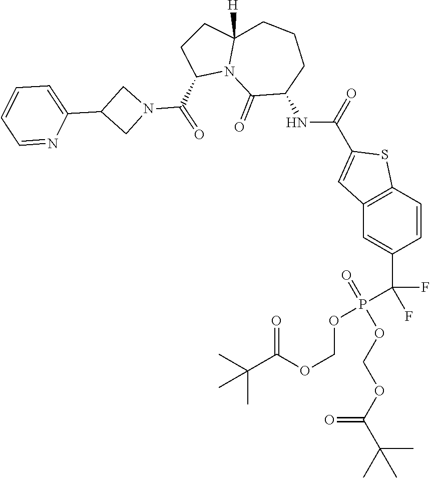 [SMILES: CC(C)(C)C(=O)OCOP(=O)(OCOC(=O)C(C)(C)C)C(F)(F)c1ccc2sc(C(=O)N[C@H]3CCC[C@H]4CC[C@@H](C(=O)N5CC(c6ccccn6)C5)N4C3=O)cc2c1] | 847.2 [M + H]⁺ | ¹H NMR (400 MHz, methanol-d₄) δ 8.69 8.60 (m, 1H), 8.09 (s, 1H), 7.95 (d, J = 8.4 Hz, 1H), 7.87-7.81 (m, 1H), 7.77-7.61 (m, 3H), 7.24- 7.18 (m, 1H), 5.88- 5.55 (m, 4H), 4.92-4.75 (m, 1H), 4.71-4.49 (m, 3H), 4.45-4.18 (m, 1H), 4.12-3.87 (m, 2H), 2.34- 1.71 (m, 11H), 1.21 (s, 18H) |
| 189 | (((difluoro(2- (((3S,6S,9aS)- 5-oxo-3-(3- (pyridin-3- yl)azetidine- 1- carbonyl) octahydro-1H- pyrrolo[1,2- a]azepin-6- yl)carbamoyl) benzo[b] thiophen-5- yl)methyl) phosphoryl)bis (oxy))bis (methylene) bis(2,2- dimethyl- propanoate) | 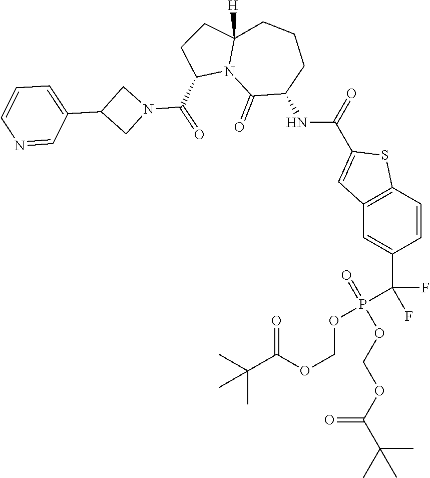 [SMILES: CC(C)(C)C(=O)OCOP(=O)(OCOC(=O)C(C)(C)C)C(F)(F)c1ccc2sc(C(=O)N[C@H]3CCC[C@H]4CC[C@@H](C(=O)N5CC(c6cccnc6)C5)N4C3=O)cc2c1] | 847.1 [M + H]⁺ | ¹H NMR (400 MHz, CDCl₃) δ 9.11 (d, J = 4.0 Hz, 1H), 8.75-8.53 (m, 1H), 8.42-8.28 (m, 1H), 8.23-8.05 (m, 2H), 7.97- 7.83 (m, 1H), 7.74- 7.55 (m, 2H), 5.79-5.58 (m, 4H), 4.94-4.79 (m, 1H), 4.76-4.62 (m, 1H), 4.51-4.39 (m, 5H), 4.26- 4.11 (m, 1H), 4.07- 3.89 (m, 2H), 2.17 (s, 2H), 2.04 (dd, J = 12.0, 5.6 Hz, 3H), 1.92 (d, J = 7.2 Hz, 3H), 1.21 (s, 18H) |
| 190 | ((((2- (((3S,6S,10aS)- 3-(rel- trans)- 3-cyano-4- phenyl- pyrrolidine- 1- carbonyl)-5- oxodecahydro- pyrrolo[1,2- a]azocin-6- yl)carbamoyl) benzo[b] thiophen-5- yl)difluoro- methyl)phos- phoryl)bis(oxy)) bis(methylene) bis(2,2- dimethyl- propanoate) | 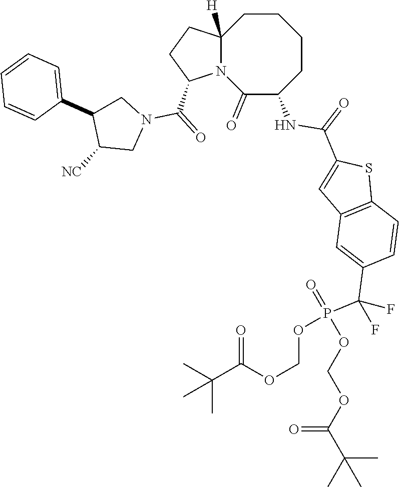 [SMILES: CC(C)(C)C(=O)OCOP(=O)(OCOC(=O)C(C)(C)C)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | 899.2 [M + H]⁺ | ¹H NMR (400 MHz, methanol-d₄) δ 8.23- 8.02 (m, 3H), 7.65-7.59 (m, 1H), 7.46-7.36 (m, 4H), 7.34 (s, 1H), 5.80- 5.66 (m, 4H), 5.05 (d, J = 10.4 Hz, 1H), 4.70 (d, J = 8.8 Hz, 1H), 4.58-4.39 (m, 2H), 4.20-4.07 (m, 1H), 4.01-3.81 (m, 1H), 3.78-3.69 (m, 1H), 3.65- 3.52 (m, 2H), 2.39- 2.19 (m, 2H), 2.10-1.96 (m, 6H), 1.92-1.78 (m, 2H), 1.75-1.59 (m, 2H), 1.18 (s, 18H) |
| 191 | (((difluoro(2- (((3S,6S,10aS)- 5-oxo-3- (6-(2- phenylacetyl)- 2,6- diazaspiro[3. 3]heptane-2- carbonyl) decahydro- pyrrolo [1,2- a]azocin-6- yl)carbamoyl) benzo[b] thiophen-5- yl)methyl) phosphoryl)bis (oxy))bis (methylene) bis(2,2- dimethyl- propanoate) | 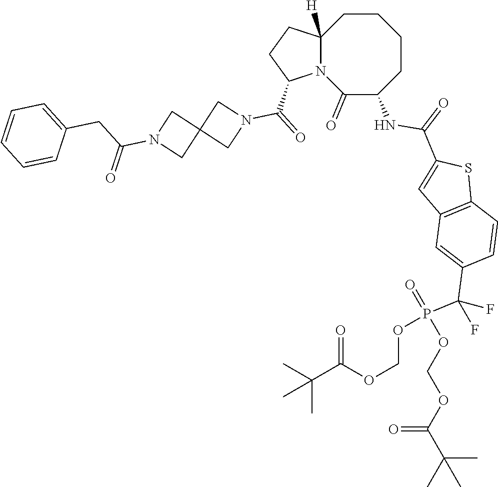 [SMILES: CC(C)(C)C(=O)OCOP(=O)(OCOC(=O)C(C)(C)C)C(F)(F)c1ccc2sc(C(=O)N[C@H]3CCCC[C@H]4CC[C@@H](C(=O)N5CC6(CN(C(=O)Cc7ccccc7)C6)C5)N4C3=O)cc2c1] | 943.3 [M + H]⁺ | ¹H NMR (400 MHz, CDCl₃) δ 8.08 (s, 1H), 7.93 (d, J = 8.4 Hz, 1H), 7.82 (s, 1H), 7.63 (d, J = 8.8 Hz, 1H), 7.36-7.29 (m, 2H), 7.25 ( s, 3H), 5.78-5.62 (m, 4H), 5.17- 5.05 (m, 1H), 4.70 (dd, J = 16.0, 8.8 Hz, 1H), 4.37- 4.12 (m, 8H), 4.10- 3.98 (m, 1H), 3.48 (d, J = 4.4 Hz, 2H), 2.18-2.04 (m, 4H), 2.04-1.87 (m, 3H), 1.87-1.76 (m, 2H), 1.71-1.68 (m, 1H), 1.67- 1.59 (m, 2H), 1.21 (s, 18H) |
| 192 | ((((2- (((3S,6S,10aS)- 3-(4-(1H- pyrazole-4- carbonyl) piperazine-1- carbonyl)-5- oxodecahydro- pyrrolo[1,2- a]azocin-6- yl)carbamoyl) benzo[b] thiophen-5- yl)difluoro- methyl)phos- phoryl)bis(oxy)) bis(methylene) bis(2,2- dimethyl- propanoate) | 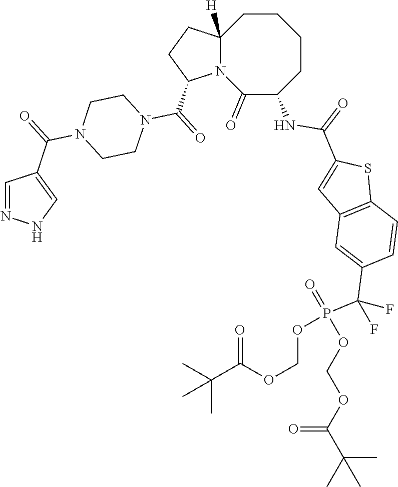 [SMILES: CC(C)(C)C(=O)OCOP(=O)(OCOC(=O)C(C)(C)C)C(F)(F)c1ccc2sc(C(=O)N[C@H]3CCCC[C@H]4CC[C@@H](C(=O)N5CCN(C(=O)c6cn[nH]c6)CC5)N4C3=O)cc2c1] | 907.1 [M + H]⁺ | ¹H NMR (400 MHz, acetonitrile-d₃) δ 8.08 (s, 1H), 8.01 (d, J = 8.4 Hz, 1H), 7.96 (s, 1H), 7.79- 7.74 (m, 2H), 7.57 (d, J = 8.4 Hz, 1H), 5.71-5.58 (m, 4H), 5.03 (m, 1H), 4.82 (t, J = 8.8 Hz, 1H), 4.37-4.24 (m, 1H), 3.78- 3.54 (m, 8H), 2.23 (s, 1H), 2.16-2.13 (m, 2H), 2.04-1.97 (m, 2H), 1.91- 1.87 (m, 1H), 1.77 (dd, J = 6.4, 11.6 Hz, 2H), 1.72- 1.66 (m, 1H), 1.60 (dd, J = 12.4, 6.8 Hz, 2H), 1.32- 1.25 (m, 1H), 1.15 (s, 18H) |
| 193 | ((((7- (((3S,6S,10aS)- 3- ((3S,4′S)-4′- cyano-2- oxospiro [indoline-3,3′- pyrrolidine]- 1′-carbonyl)- 5- oxodecahydro- pyrrolo[1,2- a]azocin-6- yl)carbamoyl) naphthalen- 2- yl)difluoro- methyl)phos- phoryl)bis(oxy)) bis(methylene) bis(2,2- dimethyl- propanoate) or ((((7- (((3S,6S,10aS)- 3- ((3R,4′R)-4′- | 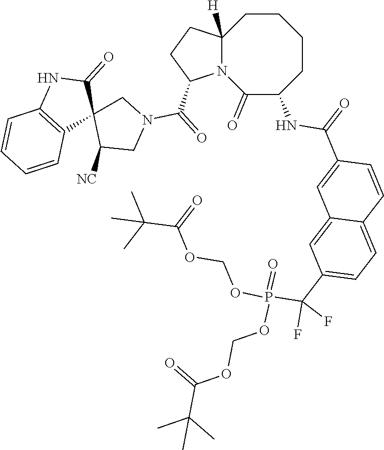 [SMILES: CC(C)(C)C(=O)OCOP(=O)(OCOC(=O)C(C)(C)C)C(F)(F)c1ccc2ccc(C(=O)N[C@H]3CCCC[C@H]4CC[C@@H](C(=O)N5C[C@@H](C#N)[C@]6(C5)C(=O)Nc5ccccc56)N4C3=O)cc2c1] or | 934.5 [M + H]⁺ | ¹H NMR (400 MHz, CDCl₃) δ 8.45-8.26 (m, 1H), 8.25-8.14 (m, 1H), 8.00-7.90 (m, 3H), 7.82- 7.65 (m, 2H), 7.43- 7.33 (m, 2H), 7.25-7.20 (m, 1H), 7.04-6.98 (m, 1H), 5.78-5.72 (m, 2H), 5.71-5.64 (m, 2H), 5.25- 5.09 (m, 1H), 4.55- 4.27 (m, 3H), 4.18-4.00 (m, 1H), 3.99-3.91 (m, 1H), 3.91-3.78 (m, 1H), 3.65 (t, J = 9.2 Hz, 1H), 2.28-2.12 (m, 4H), 2.11- 1.90 (m, 4H), 1.85- 1.68 (m, 4H), 1.21 (s, 18H) |
| | cyano-2- oxospiro [indoline-3,3′- pyrrolidine]- 1′-carbonyl)- 5- oxodecahydro- pyrrolo[1,2- a]azocin-6- yl)carbamoyl) naphthalen- 2- yl)difluoro- methyl)phos- phoryl)bis(oxy)) bis(methylene) bis(2,2- dimethyl- propanoate) or ((((7- (((3S,6S,10aS)- 3- ((3R,4′S)-4′- cyano-2- oxospiro [indoline-3,3′- pyrrolidine]- | 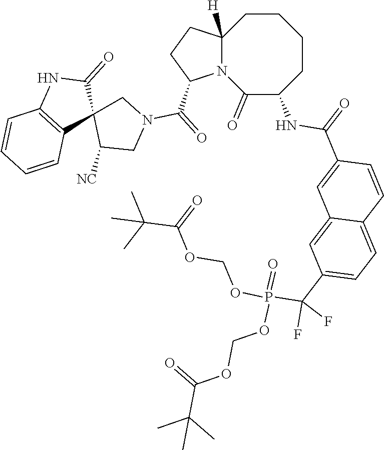 [SMILES: CC(C)(C)C(=O)OCOP(=O)(OCOC(=O)C(C)(C)C)C(F)(F)c1ccc2ccc(C(=O)N[C@H]3CCCC[C@H]4CC[C@@H](C(=O)N5C[C@H](C#N)[C@@]6(C5)C(=O)Nc5ccccc56)N4C3=O)cc2c1] or | | |
| | 1′-carbonyl)- 5- oxodecahydro- pyrrolo[1,2- a]azocin-6- yl)carbamoyl) naphthalen- 2- yl)difluoro- methyl)phos- phoryl)bis(oxy)) bis(methylene) bis(2,2- dimethyl- propanoate) or ((((7- (((3S,6S,10aS)- 3- ((3S,4′R)-4′- cyano-2- oxospiro [indoline-3,3′- pyrrolidine]- 1′-carbonyl)- 5- oxodecahydro- pyrrolo[1,2- | 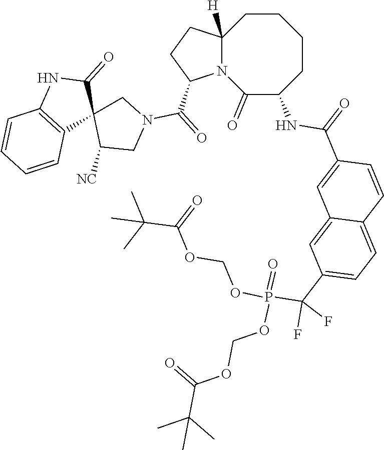 [SMILES: CC(C)(C)C(=O)OCOP(=O)(OCOC(=O)C(C)(C)C)C(F)(F)c1ccc2ccc(C(=O)N[C@H]3CCCC[C@H]4CC[C@@H](C(=O)N5C[C@H](C#N)[C@]6(C5)C(=O)Nc5ccccc56)N4C3=O)cc2c1] or | | |
| | a]azocin-6- yl)carbamoyl) naphthalen- 2- yl)difluoro- methyl)phos- phoryl)bis(oxy)) bis(methylene) bis(2,2- dimethyl- propanoate) | 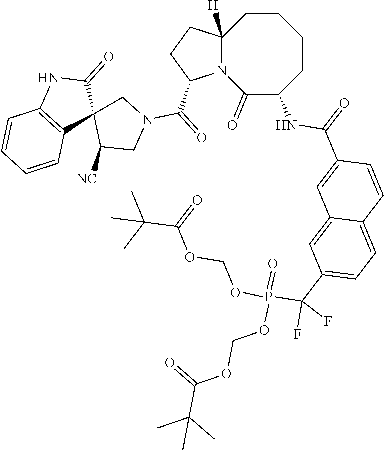 [SMILES: CC(C)(C)C(=O)OCOP(=O)(OCOC(=O)C(C)(C)C)C(F)(F)c1ccc2ccc(C(=O)N[C@H]3CCCC[C@H]4CC[C@@H](C(=O)N5C[C@@H](C#N)[C@@]6(C5)C(=O)Nc5ccccc56)N4C3=O)cc2c1] (SFC Peak 2 of pyrrolidine building block used for synthesis and biological testing) | | |
| 194 | ((((7- (((3S,6S,10aS)- 3-(rel- (trans)- 3-cyano-4- phenyl- pyrrolidine-1- carbonyl)-5- oxodecahydro- pyrrolo[1,2- a]azocin-6- yl)carbamoyl) naphthalen- 2- yl)difluoro- methyl)phos- phoryl)bis(oxy)) bis(methylene) bis(2,2- dimethyl- propanoate) | 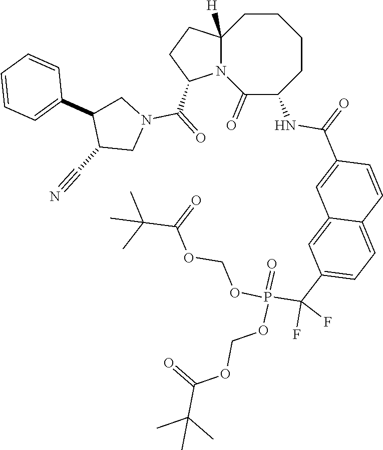 [SMILES: CC(C)(C)C(=O)OCOP(=O)(OCOC(=O)C(C)(C)C)C(F)(F)c1ccc2ccc(C(=O)N[C@H]3CCCC[C@H]4CC[C@@H](C(=O)N5C[C@H](c6ccccc6)[C@@H](C#N)C5)N4C3=O)cc2c1] | 893.3 [M + H]⁺ | ¹H NMR (400 MHz, methanol-d₄) δ 8.64- 8.52 (m, 1H), 8.26 (s, 1H), 8.13-8.02 (m, 3H), 7.74 (d, J = 8.4 Hz, 1H), 7.50-7.26 (m, 5H), 5.82- 5.67 (m, 4H), 5.15- 5.06 (m, 1H), 4.74-4.63 (m, 1H), 4.56 (s, 1H), 4.52-4.44 (m, 1H), 4.22- 4.06 (m, 1H), 4.02- 3.79 (m, 1H), 3.79-3.66 (m, 1H), 3.65-3.46 (m, 2H), 2.41-2.23 (m, 2H), 2.13-1.96 (m, 6H), 1.93- 1.79 (m, 2H), 1.76- 1.62 (m, 2H), 1.35-1.12 (m, 18H) |
| 195 | isopropyl (((2- (((3S,6S,9aS)- 5-oxo-3-(3- (pyridin-3- yl)azetidine- 1- carbonyl)octa- hydro-1H- pyrrolo[1,2- a]azepin-6- yl)carbamoyl) benzo[b] thiophen-5- yl)methyl) (phenoxy) phosphoryl)-L- alaninate | 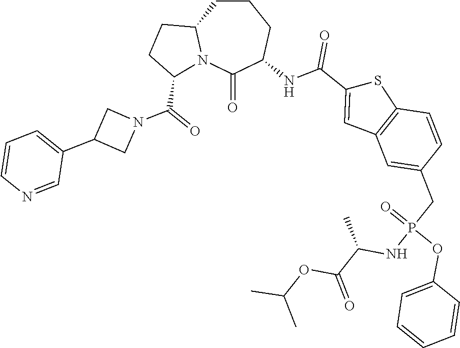 [SMILES: CC(C)OC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 772.4 [M + H]⁺ | ¹H NMR (400 MHz, DMSO-d₆) δ 8.84-8.70 (m, 1H), 8.61-8.52 (m, 1H), 8.51-8.39 (m, 1H), 8.21 (s, 1H), 7.98-7.82 (m, 3H), 7.48-7.27 (m, 4H), 7.19-7.08 (m, 3H), 5.74-5.48 (m, 1H), 4.88- 4.52 (m, 3H), 4.46- 4.16 (m, 3H), 4.04-3.70 (m, 4H), 3.53-3.39 (m, 2H), 2.27-2.12 (m, 1H), 2.08-1.92 (m, 2H), 1.90- 1.59 (m, 7H), 1.14- 0.95 (m, 9H) |
| 196 | isopropyl ((fluoro(2- (((3S,6S,9aS)- 5-oxo-3-(3- (pyridin-3- yl)azetidine- 1- carbonyl) octahydro-1H- pyrrolo[1,2- a]azepin-6- yl)carbamoyl) benzo[b] thiophen-5- yl)methyl) (phenoxy) phosphoryl)-L- alaninate | 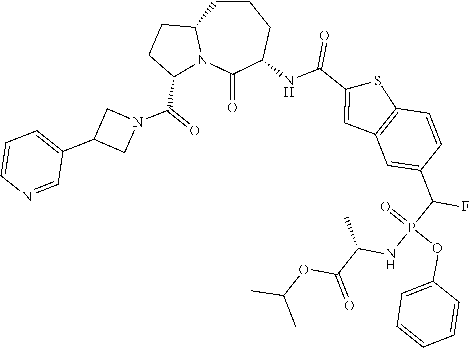 [SMILES: CC(C)OC(=O)[C@H](C)NP(=O)(Oc1ccccc1)C(F)c1ccc2sc(C(=O)N[C@H]3CCC[C@H]4CC[C@@H](C(=O)N5CC(c6cccnc6)C5)N4C3=O)cc2c1] | 790.8 [M + H]⁺ | ¹H NMR (400 MHz, DMSO-d₆) δ 8.72-8.45 (m, 2H), 8.04-7.64 (m, 5H), 7.63-7.51 (m, 1H), 7.45-7.29 (m, 2H), 7.27- 7.02 (m, 4H), 6.07- 5.85 (m, 1H), 5.05-4.91 (m, 1H), 4.75-4.34 (m, 4H), 4.24-3.05 (m, 6H), 2.34-2.15 (m, 2H), 2.12- 1.87 (m, 6H), 1.86- 1.68 (m, 2H), 1.42-1.08 (m, 9H) |
| 197 | isopropyl (((2- (((3S,6S,10aS)- 3-((3R,4S or 3S,4R)-3- cyano-4-(2- oxo-1,2- dihydro- pyridin-4- yl)pyrrolidine- 1- carbonyl)-5- oxodecahydro- pyrrolo[1,2- a]azocin-6- yl)carbamoyl) benzo[b] thiophen-5- yl)methyl) (phenoxy) phosphoryl)-L- alaninate | 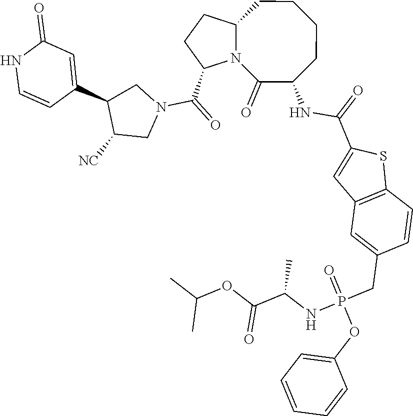 [SMILES: CC(C)OC(=O)[C@H](C)NP(=O)(Cc1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1)Oc1ccccc1] or | 841.3 [M + H]⁺ | ¹H NMR (400 MHz, DMSO-d₆) δ 11.60 (s, 1H), 8.87-8.67 (m, 1H), 8.25-8.15 (m, 1H), 8.00- 7.90 (m, 1H), 7.90- 7.83 (m, 1H), 7.48-7.36 (m, 2H), 7.36-7.26 (m, 2H), 7.18-7.09 (m, 3H), 6.41-6.30 (m, 1H), 6.29- 6.17 (m, 1H), 5.71- 5.50 (m, 1H), 4.99-4.88 (m, 1H), 4.85-4.73 (m, 1H), 4.59-4.50 (m, 1H), 4.36-4.19 (m, 2H), 3.95- 3.42 (m, 7H), 3.24- 3.15 (m, 1H), 2.34-2.18 (m, 1H), 2.10-2.02 (m, 1H), 1.92-1.74 (m, 6H), 1.64-1.51 (m, 2H), 1.24- 1.15 (m, 1H), 1.14- 0.94 (m, 10H) |
| | | 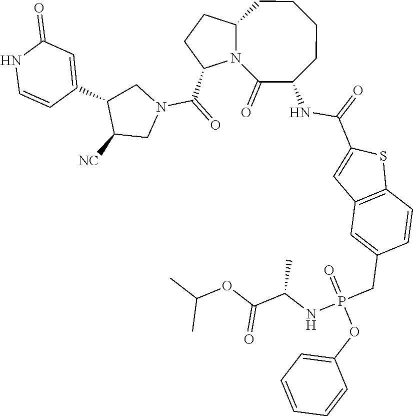 [SMILES: CC(C)OC(=O)[C@H](C)NP(=O)(Cc1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1)Oc1ccccc1] SFC peak 2 was used for biological testing | | |
| 198 | isopropyl (((2- (((3S,6S,10aS)- 3-((3R,4S or 3S,4R)-3- cyano-4-(2- oxo-1,2- dihydropyridin- 4- yl)pyrrolidine- 1- carbonyl)-5- oxodecahydro- pyrrolo[1,2- a]azocin-6- yl)carbamoyl) benzo[b] thiophen-5- yl)fluoro- methyl) (phenoxy) phosphoryl)- L-alaninate | 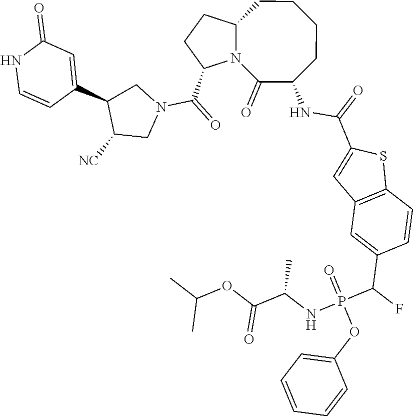 [SMILES: CC(C)OC(=O)[C@H](C)NP(=O)(Oc1ccccc1)C(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] or | 859.2 [M + H]⁺ | ¹H NMR (400 MHz, CDCl₃) δ 7.98-7.48 (m, 5H), 7.37-7.27 (m, 2H), 7.25-6.95 (m, 4H), 6.57- 6.39 (m, 1H), 6.17- 5.84 (m, 2H), 5.20-5.07 (m, 1H), 5.06-4.88 (m, 1H), 4.63-4.42 (m, 2H), 4.39-4.28 (m, 1H), 4.21- 3.51 (m, 5H), 3.49- 3.37 (m, 1H), 3.32-3.07 (m, 1H), 2.26-1.73 (m, 10H), 1.69-1.57 (m, 2H), 1.29-1.09 (m, 9H) |
| | | 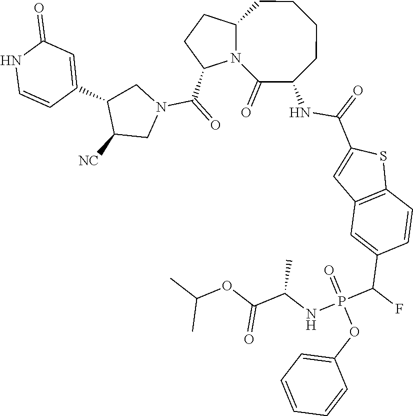 [SMILES: CC(C)OC(=O)[C@H](C)NP(=O)(Oc1ccccc1)C(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] SFC peak 1 was used for biological testing | | |

### S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-hydroxy-2,2-dimethylpropanethioate)trifluoroacetate (199)

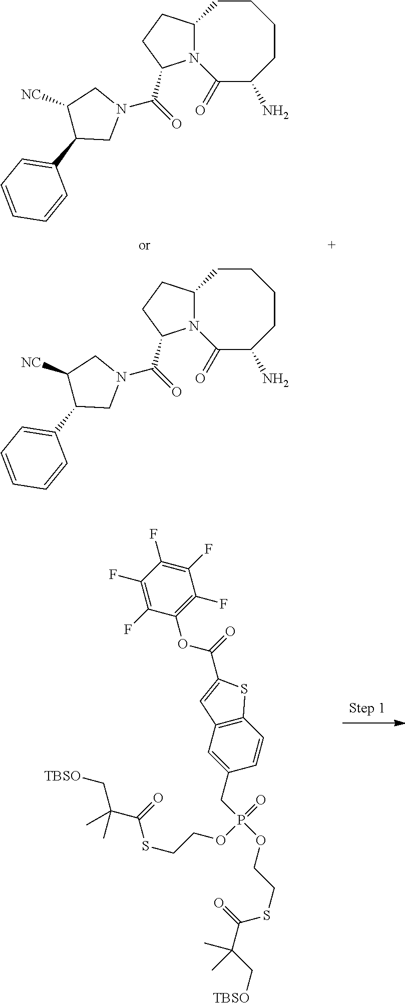 [SMILES: N#C[C@@H]1CN(C(=O)[C@@H]2CC[C@@H]3CCCC[C@H](N)C(=O)N32)C[C@H]1c1ccccc1 ; N#C[C@H]1CN(C(=O)[C@@H]2CC[C@@H]3CCCC[C@H](N)C(=O)N32)C[C@@H]1c1ccccc1 ; CC(C)(CO[Si](C)(C)C(C)(C)C)C(=O)SCCOP(=O)(Cc1ccc2sc(C(=O)Oc3c(F)c(F)c(F)c(F)c3F)cc2c1)OCCSC(=O)C(C)(C)CO[Si](C)(C)C(C)(C)C]

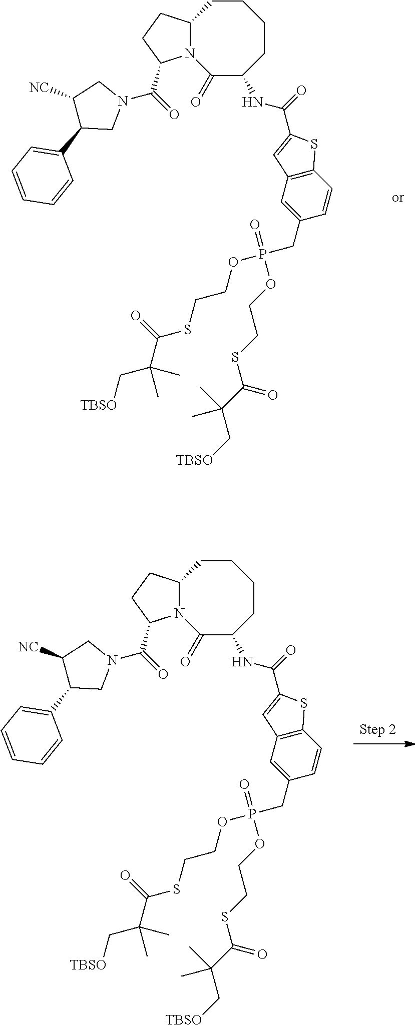 [SMILES: CC(C)(CO[Si](C)(C)C(C)(C)C)C(=O)SCCOP(=O)(Cc1ccc2sc(C(=O)N[C@H]3CCCC[C@H]4CC[C@@H](C(=O)N5C[C@@H](C#N)[C@H](c6ccccc6)C5)N4C3=O)cc2c1)OCCSC(=O)C(C)(C)CO[Si](C)(C)C(C)(C)C ; CC(C)(CO[Si](C)(C)C(C)(C)C)C(=O)SCCOP(=O)(Cc1ccc2sc(C(=O)N[C@H]3CCCC[C@H]4CC[C@@H](C(=O)N5C[C@H](c6ccccc6)[C@@H](C#N)C5)N4C3=O)cc2c1)OCCSC(=O)C(C)(C)CO[Si](C)(C)C(C)(C)C]

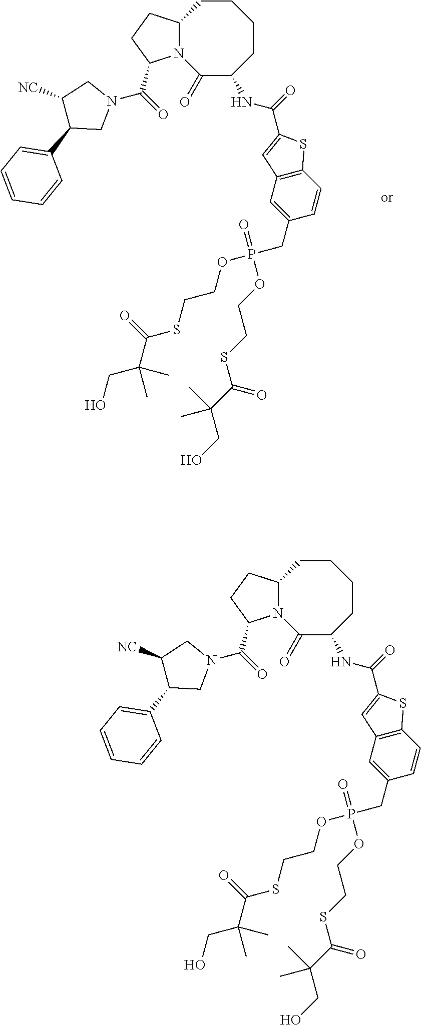 [SMILES: CC(C)(CO)C(=O)SCCOP(=O)(Cc1ccc2sc(C(=O)N[C@H]3CCCC[C@H]4CC[C@@H](C(=O)N5C[C@@H](C#N)[C@H](c6ccccc6)C5)N4C3=O)cc2c1)OCCSC(=O)C(C)(C)CO ; CC(C)(CO)C(=O)SCCOP(=O)(Cc1ccc2sc(C(=O)N[C@H]3CCCC[C@H]4CC[C@@H](C(=O)N5C[C@H](c6ccccc6)[C@@H](C#N)C5)N4C3=O)cc2c1)OCCSC(=O)C(C)(C)CO]

### Step 1: Preparation of S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-((tert-butyldimethylsilyl)oxy)-2,2-dimethylpropanethioate)

S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-((tert-butyldimethylsilyl)oxy)-2,2-dimethylpropanethioate) was prepared using the procedure described for the synthesis of S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))dibutanethioate and starting from perfluorophenyl 5-((bis(2-((3-((tert-butyldimethylsilyl)oxy)-2,2-dimethylpropanoyl)thio)ethoxy)phosphoryl)methyl)benzo[b]thiophene-2-carboxylate and (3S,4R or 3R,4S)-1-((3S,6S,10aS)-6-amino-5-oxodecahydropyrrolo[1,2-a]azocine-3-carbonyl)-4-phenylpyrrolidine-3-carbonitrile (SFC peak 2 was used). The product was purified using standard chromatographic techniques.

The reaction with perfluorophenyl 5-((bis(2-(butyrylthio)ethoxy)phosphoryl)methyl)benzo[b]thiophene-2-carboxylate (25.0 mg, 25.3 μmol, 1.0 eq) and (3S,4R or 3R,4S)-1-((3S,6S,10aS)-6-amino-5-oxodecahydropyrrolo[1,2-a]azocine-3-carbonyl)-4-phenylpyrrolidine-3-carbonitrile (9.62 mg, 25.3 μmol, 1.0 eq) afforded S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-((tert-butyldimethylsilyl)oxy)-2,2-dimethylpropanethioate) (14.0 mg, 11.8 μmol, 46.8% yield) as a colorless oil after prep-HPLC purification.

### Step 2: Preparation of S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-hydroxy-2,2-dimethylpropanethioate)trifluoroacetate (199)

To a solution of S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-((tert-butyldimethylsilyl)oxy)-2,2-dimethylpropanethioate) (14 mg, 11.8 μmol, 1.0 eq) in CH2Cl2 (0.5 mL) added TFA (13.4 mg, 118 μmol, 10.0 eq). The reaction mixture was stirred at 25° C. for 0.5 h and subsequently concentrated in vacuo. The crude product was pre-purified by column chromatography followed by prep-HPLC purification to afford S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-hydroxy-2,2-dimethylpropanethioate)trifluoroacetate (199) (4.90 mg, 5.13 μmol, 43.7% yield). LCMS ESI (m/z)=955.0 [M+H]⁺; ¹H NMR (400 MHz, DMSO-d₆) δ 8.77 (m, 1H), 8.21 (m, 1H), 7.98-7.91 (m, 1H), 7.84 (s, 1H), 7.41 (m, 5H), 7.34-7.26 (m, 1H), 5.08-4.91 (m, 1H), 4.54 (m, 1H), 4.29 (m, 1H), 3.98 (m, 4H), 3.84-3.62 (m, 3H), 3.47-3.38 (m, 8H), 3.04 (m, 4H), 2.30-2.22 (m, 1H), 1.84 (m, 6H), 1.59 (m, 3H), 1.24 (s, 2H), 1.10 (s, 12H)

The following compounds in Table 38 were prepared using the representative protocol described above for the synthesis S,S′-(((((2-(((3S,6S,10aS)-3-((3S,4R or 3R,4S)-3-cyano-4-phenylpyrrolidine-1-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-hydroxy-2,2-dimethylpropanethioate)trifluoroacetate (199) and utilizing the appropriate starting materials and modifications.

TABLE 38

| Compound | Name | Structure | LCMS | NMR |
|---|---|---|---|---|
| 200 | S,S′-(((((2- (((3S,6S,10a S)-3-((3S,4R or 3R,4S)-3- cyano-4- phenylpyrrolidine- 1- carbonyl)-5- oxodecahydro- pyrrolo [1,2-a]azocin-6- yl)carbamoyl) benzo[b] thiophen-5-yl) difluoromethyl) phosphoryl) bis(oxy)) bis(ethane- 2,1-diyl)) bis(3- hydroxy-2,2- dimethyl- propanethioate) | 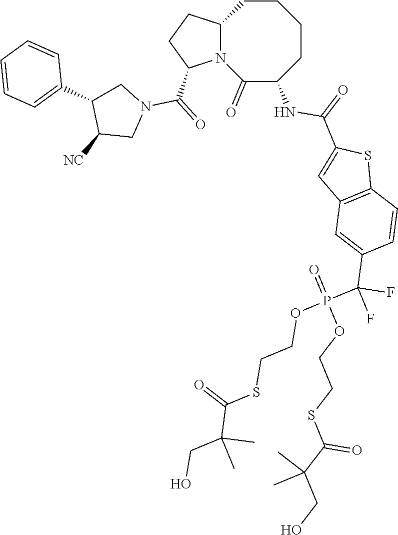 [SMILES: CC(C)(CO)C(=O)SCCOP(=O)(OCCSC(=O)C(C)(C)CO)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 990.8 [M + H]⁺ | ¹H NMR (400 MHz, CDCl₃) δ 8.16 − 8.02 (m, 1H), 8.00 − 7.85 (m, 2H), 7.61 (d, J = 8.5 Hz, 1H), 7.45 − 7.29 (m, 6H), 5.23 − 5.09 (m, 1H), 4.61 − 4.49 (m, 1H), 4.37 − 3.95 (m, 8H), 3.79 − 3.47 (m, 6H), 3.35 − 3.03 (m, 5H), 2.25 − 2.17 (m, 3H), 2.15 − 2.10 (m, 3H), 2.03 − 2.00 (m, 1H), 1.90 − 1.75 (m, 3H), 1.72 − 1.61 (m, 2H), 1.25 − 1.14 (m, 12H) |
| | | or | | |
| | | 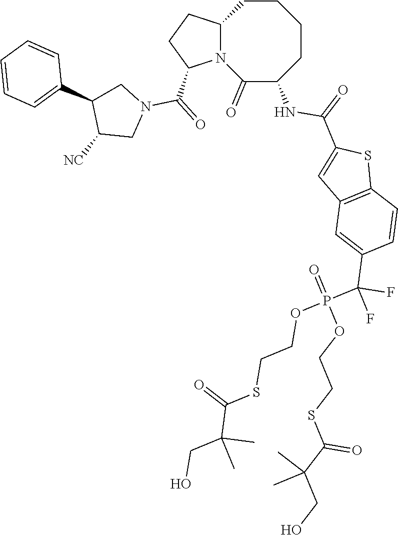 [SMILES: CC(C)(CO)C(=O)SCCOP(=O)(OCCSC(=O)C(C)(C)CO)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |
| 201 | S-(2- (((difluoro(2- (((3S,6S,9aS)- 5-oxo-3-(3- (pyridin-3- yl)azetidine-1- carbonyl) octahydro-1H- pyrrolo[1,2- a]azepin-6- yl)carbamoyl) benzo[b]thiophen- 5-yl)methyl) (phenoxy) phosphoryl)oxy) ethyl) 3- hydroxy-2,2- dimethyl- propanethioate | 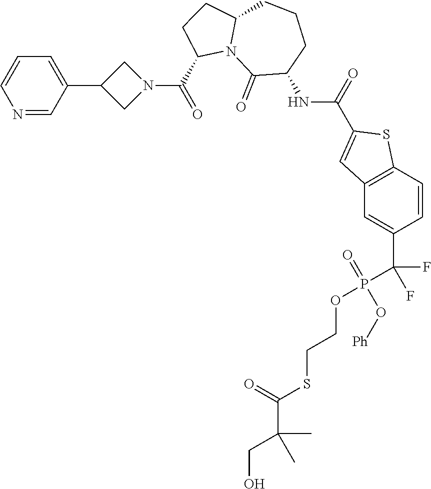 [SMILES: CC(C)(CO)C(=O)SCCOP(=O)(Oc1ccccc1)C(F)(F)c1ccc2sc(C(=O)N[C@H]3CCC[C@H]4CC[C@@H](C(=O)N5CC(c6cccnc6)C5)N4C3=O)cc2c1] | 854.9 [M + H]⁺ | ¹H NMR (400 MHz, CDCl₃) δ 8.87 − 8.51 (m, 2H), 8.15 (d, J = 16.3 Hz, 1H), 8.05 − 7.84 (m, 3H), 7.77 − 7.63 (m, 2H), 7.48 − 7.29 (m, 3H), 7.22 7.12 (m, 3H), 5.02 − 4.44 (m, 5H), 4.32 − 4.16 (m, 3H), 4.09 − 3.88 (m, 2H), 3.53 (s, 2H), 3.16 − 2.99 (m, 2H), 2.32 − 2.16 (m, 3H), 2.09 − 2.04 (m, 3H), 1.96 − 1.92 (m, 2H), 1.85 − 1.79 (m, 2H), 1.17 (dd, J = 5.8, 2.4 Hz, 6H) |
| 202 | S,S′-(((((2- (((3S,6S,10a S)-3-((3S,4R or 3R,4S)-3- cyano-4- phenylpyrrolidine- 1-carbonyl)-5- oxodecahydro- pyrrolo [1,2- a]azocin-6-yl) carbamoyl)benzo [b]thiophen-5- yl)difluoromethyl) phosphoryl) bis(oxy)) bis(ethane- 2,1-diyl)) bis(3- hydroxy-3- methylbutane thioate) | 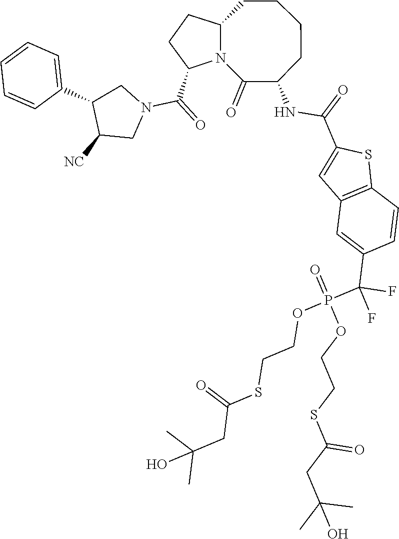 [SMILES: CC(C)(O)CC(=O)SCCOP(=O)(OCCSC(=O)CC(C)(C)O)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 991.3 [M + H]⁺ | ¹H NMR (400 MHz, DMSO-d₆) δ 9.01 − 8.79 (m, 1H), 8.35 (d, J = 10.1 Hz, 1H), 8.28 − 8.07 (m, 2H), 7.60 (s, 1H), 7.39 (m, 5H), 4.96 (s, 1H), 4.66 (s, 1H), 4.60 − 4.48 (m, 1H), 4.39 − 3.91 (m, 8H), 3.88 − 3.77 (m, 1H), 3.70 − 3.50 (m, 2H), 3.12 (s, 4H), 2.66 (s, 4H), 2.27 (s, 1H), 2.07 − 1.54 (m, 11H), 1.17 (s, 12H) |
| | | or | | |
| | | 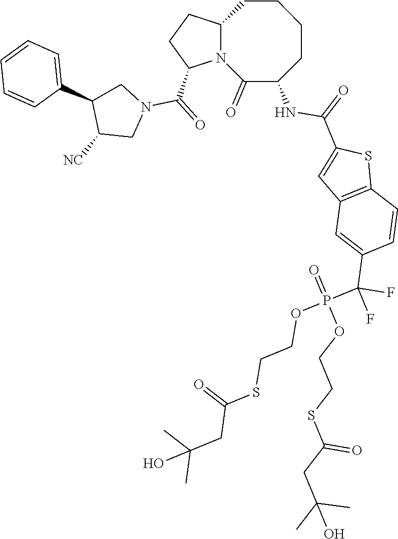 [SMILES: CC(C)(O)CC(=O)SCCOP(=O)(OCCSC(=O)CC(C)(C)O)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] | | |
| | | SFC peak 2 was used for | | |
| | | biological testing | | |

### List of SFC Methods

A listing of certain SFC conditions are shown in Table 39.

TABLE 39

| | Preparative SFC | |
|---|---|---|
| | conditions for | |
| Method | separation | Used for |
| 1 | Lux i-Cellulose-5 | methyl (3S,6S,10aR)-6-((tert-butoxycarbonyl)amino)- |
| | 21.2 × 250 mm 5 um | 9-hydroxy-5-oxodecahydropyrrolo[1,2-a]azocine-3- |
| | column, column | carboxylate and methyl (3S,6S,10aR)-6-((tert- |
| | temp = 40° C., flow | butoxycarbonyl)amino)-8-hydroxy-5- |
| | rate 75 mL/min, 20% | oxodecahydropyrrolo[1,2-a]azocine-3-carboxylate |
| | MeOH, cycle time: 5 | |
| | min | |
| 2 | ChiralCel OX, | tert-butyl ((1aS,3S,6S,8aR,9aR)-4-oxo-6-((S)-3- |
| | 250 × 21.2 mm I.D., | phenylpyrrolidine-1-carbonyl)decahydrooxireno[2,3- |
| | 5 μm, A for CO₂ and | d]pyrrolo[1,2-a]azocin-3-yl)carbamate and tert-butyl |
| | B for MeOH + 0.1% | ((1aS,3S,6S,8aR,9aR)-4-oxo-6-((R)-3- |
| | NH₄OH, 40 mL/min | phenylpyrrolidine-1-carbonyl)decahydrooxireno[2,3- |
| | | d]pyrrolo[1,2-a]azocin-3-yl)carbamate |
| 3 | ChiralPak R,R- | tert-butyl ((3S,6S,8R,9R,10aR)-9-hydroxy-8-methyl-5- |
| | WHELK, | oxo-3-((S)-3-phenylpyrrolidine-1- |
| | 250 × 21.2 mm I.D., | carbonyl)decahydropyrrolo[1,2-a]azocin-6- |
| | 5 μm; Mobile phase: | yl)carbamate and tert-butyl ((3S,6S,8S,9S,10aR)-8- |
| | A for CO₂ and B for | hydroxy-9-methyl-5-oxo-3-((S)-3-phenylpyrrolidine-1- |
| | MeOH + 0.1% | carbonyl)decahydropyrrolo[1,2-a]azocin-6- |
| | NH₄OH Gradient: B | yl)carbamate |
| | 25%; flow rate: 40 | |
| | mL/min | |
| 4 | Lux i-Amylose 3, | benzyl (R)-5- |
| | 21.2 × 250 mm 5 μm | ((diethoxyphosphoryl)fluoromethyl)benzo[b]thiophene- |
| | column, 75 mL/min, | 2-carboxylate and benzyl (S)-5- |
| | 40% MeOH | ((diethoxyphosphoryl)fluoromethyl)benzo[b]thiophene- |
| | | 2-carboxylate |
| 5 | DAICEL | tert-butyl ((3S,6S,8R,9aR)-8-methyl-5-oxo-3-(3- |
| | CHIRALPAK AD | (pyridin-3-yl)azetidine-1-carbonyl)octahydro-1H- |
| | 250 mm × 30 mm, | pyrrolo[1,2-a]azepin-6-yl)carbamate and tert-butyl |
| | 10 um, Mobile | ((3S,6S,8S,9aR)-8-methyl-5-oxo-3-(3-(pyridin-3- |
| | phase: Phase A for | yl)azetidine-1-carbonyl)octahydro-1H-pyrrolo[1,2- |
| | CO₂, and Phase B for | a]azepin-6-yl)carbamate |
| | Neu-IPA; Gradient | |
| | elution: B in A 30% | |
| 6 | ChiralPak C-IG, 100 × | Trans pyrrolidines: |
| | 4.6 mm I.D., 5 μm; | tert-butyl (3R,4S)-3-cyano-4-phenylpyrrolidine-1- |
| | Mobile phase: A for | carboxylate |
| | CO₂ and B for | tert-butyl (3S,4R)-3-cyano-4-phenylpyrrolidine-1- |
| | methanol (0.05% | carboxylate |
| | diethylamine); | tert-butyl (3R,4S)-3-cyano-4-(2-oxo-1,2- |
| | Gradient: 10% to | dihydropyridin-4-yl)pyrrolidine-1-carboxylate |
| | 40% B in 8 min; | tert-butyl (3S,4R)-3-cyano-4-(2-oxo-1,2- |
| | Flow rate: 2.5 | dihydropyridin-4-yl)pyrrolidine-1-carboxylate |
| | mL/min; Back | |
| | pressure: 100 bar; | |
| | Column temperature: | |
| | 40° C.; Wavelength: | |
| | 210 nm; Cycle-time: | |
| | 2 min | |
| 7 | Column Lux- | (3S,4′S)-2-oxospiro[indoline-3,3′-pyrrolidine]-4′- |
| | Cellulose-5 21.2 × | carbonitrile and (3R,4′R)-2-oxospiro[indoline-3,3′- |
| | 250 mm, 5 um | pyrrolidine]-4′-carbonitrile or (3S,4′R)-2- |
| | column, 3.7 mg/inj, | oxospiro[indoline-3,3′-pyrrolidine]-4′-carbonitrile or |
| | concentration 36.9 | (3R,4′S)-2-oxospiro[indoline-3,3′-pyrrolidine]-4′- |
| | mg/mL, Column T = | carbonitrile |
| | 40° C., Flow rate 75 | |
| | mL/min, 15% MeOH | |
| | (with 0.1% | |
| | diethylamine), cycle | |
| | time: 4.9 min | |

### Further Compounds and Methods

### Procedures for Syntheses of Cores:

### Synthesis of methyl (1R,3S,6S,10aS,Z)-6-((tert-butoxycarbonyl)amino)-1-hydroxy-5-oxo-1,2,3,5,6,7,10,10a-octahydropyrrolo[1,2-a]azocine-3-carboxylate and methyl (1R,3S,6S,10aR,Z)-6-((tert-butoxycarbonyl)amino)-1-hydroxy-5-oxo-1,2,3,5,6,7,10,10a-octahydropyrrolo[1,2-a]azocine-3-carboxylate

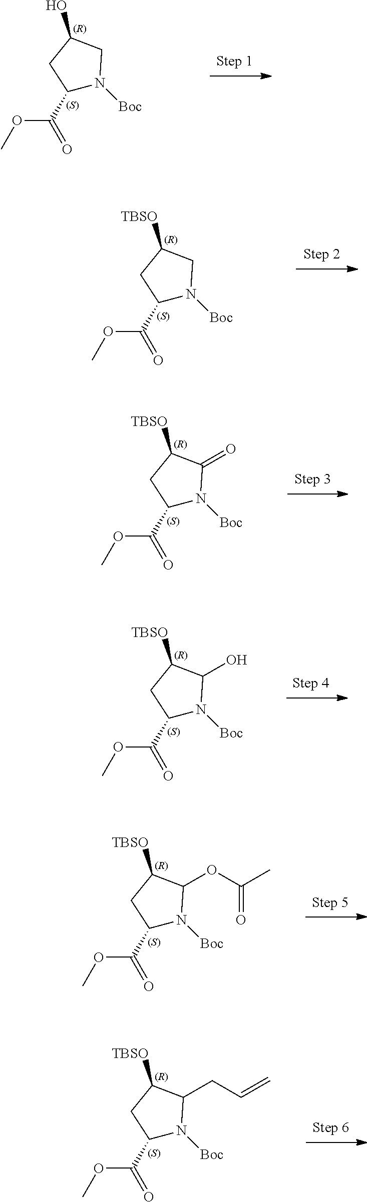

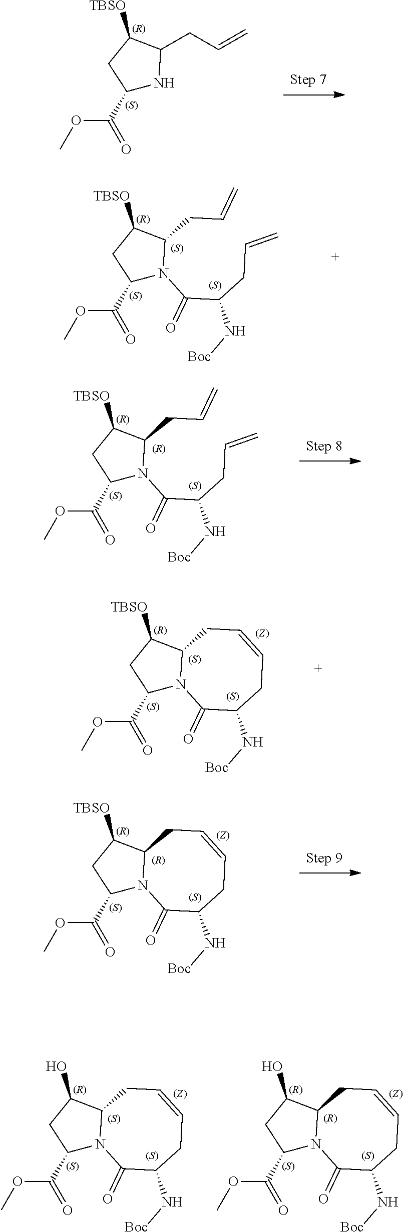

### Step 1: 1-(tert-butyl) 2-methyl (2S,4R)-4-((tert-butyldimethylsilyl)oxy)pyrrolidine-1,2-dicarboxylate

To a solution of 1-(tert-butyl) 2-methyl (2S,4R)-4-hydroxypyrrolidine-1,2-dicarboxylate (330 g, 1.35 mol, 1.00 eq) in DMF (2.50 L) was added imidazole (183 g, 2.69 mol, 2.00 eq) at 25° C. under N₂. The mixture was cooled to 0° C. and stirred for 0.50 hr. To the mixture was added TBSCl (243 g, 1.61 mol, 198 mL, 1.20 eq) at 0° C. and the mixture was stirred at 0° C. for 0.5 hr. The mixture was allowed to warm to 25° C. and stirred at 25° C. for 12 h. TLC (petroleum ether/ethyl acetate=1/3) showed that 1-(tert-butyl) 2-methyl (2S,4R)-4-hydroxypyrrolidine-1,2-dicarboxylate (R_{f}=0.50) was consumed completely and a major new spot (R_{f}=0.80) was formed. Three batches were combined. The mixture was poured into 15.0 L water and extracted with ethyl acetate (5.00 L*2). The organic layer was washed with saturated NaHCO₃(5.00 L*2), saturated NH₄Cl (5.00 L*2), brine (5.00 L), dried over Na₂SO₄, filtered and concentrated to give 1-(tert-butyl) 2-methyl (2S,4R)-4-((tert-butyldimethylsilyl)oxy)pyrrolidine-1,2-dicarboxylate (1.54 kg, crude) as colorless oil. ¹H NMR: (400 MHz, CDCl₃) δ 4.49-4.29 (m, 2H), 3.80-3.69 (m, 3H), 3.67-3.54 (m, 1H), 3.47-3.28 (m, 1H), 2.25-2.12 (m, 1H), 2.08-1.96 (m, 1H), 1.52-1.38 (m, 9H), 0.88 (s, 9H), 0.07 (s, 6H).

### Step 2: Synthesis of 1-(tert-butyl) 2-methyl (2S,4R)-4-((tert-butyldimethylsilyl)oxy)-5-oxopyrrolidine-1,2-dicarboxylate

To a solution of NaIO₄ (250 g, 1.17 mol, 64.7 mL, 3.00 eq) in H₂O (2.20 L) was added RuO₂·H₂O (11.8 g, 77.9 mmol, 0.20 eq) at 25° C. under N₂. The mixture was stirred at 25° C. for 0.50 hr. To the mixture was added a solution of 1-(tert-butyl) 2-methyl (2S,4R)-4-((tert-butyldimethylsilyl)oxy)pyrrolidine-1,2-dicarboxylate (140 g, 389 mmol, 1.00 eq) in ethyl acetate (1.20 L) at 25° C. The mixture was stirred at 25° C. for 12 h. TLC (petroleum ether/ethyl acetate=10/1) showed that 1-(tert-butyl) 2-methyl (2S,4R)-4-((tert-butyldimethylsilyl)oxy)pyrrolidine-1,2-dicarboxylate (R_{f}=0.40) was consumed and a major new spot (R_{f}=0.45) was formed. Six batches were combined. The mixture was filtered. The filtrate was extracted with ethyl acetate (10.0 L*3). The combined organic layer was washed with Na₂S₂O₃ (10.0 L*2), brine (10.0 L), dried over Na₂SO₄, filtered and concentrated to give 1-(tert-butyl) 2-methyl (2S,4R)-4-((tert-butyldimethylsilyl)oxy)-5-oxopyrrolidine-1,2-dicarboxylate (822 g, crude) as yellow oil. ¹H NMR: (400 MHz, CDCl₃) δ 4.60-4.57 (m, 1H), 4.45-4.40 (m, 1H), 3.79 (s, 3H), 2.39-2.34 (m, 1H), 2.25-2.17 (m, 1H), 1.51 (s, 9H), 0.90 (s, 9H), 0.18 (s, 3H), 0.13 (s, 3H).

### Step 3: Synthesis of 1-(tert-butyl) 2-methyl (2S,4R)-4-((tert-butyldimethylsilyl)oxy)-5-hydroxypyrrolidine-1,2-dicarboxylate

To a solution of 1-(tert-butyl) 2-methyl (2S,4R)-4-((tert-butyldimethylsilyl)oxy)-5-oxopyrrolidine-1,2-dicarboxylate (450 g, 1.20 mol, 1.00 eq) in THF (1.35 L) was added LiBHEt₃ (1 M, 1.33 L, 1.10 eq) at −78° C., then stirred at −78° C. for 3 h. TLC (petroleum ether/ethyl acetate=3/1) showed 1-(tert-butyl) 2-methyl (2S,4R)-4-((tert-butyldimethylsilyl)oxy)-5-oxopyrrolidine-1,2-dicarboxylate (R_{f}=0.60) remained and the desired spots (R_{f}=0.45, 0.50) were detected. The resulting mixture was quenched by saturated NaHCO₃(1000 mL), then extracted with ethyl acetate (1500 mL*2). The combined organic layers were dried over Na₂SO₄, filtered and concentrated under vacuum. 1-(tert-butyl) 2-methyl (2S,4R)-4-((tert-butyldimethylsilyl)oxy)-5-hydroxypyrrolidine-1,2-dicarboxylate (450 g, Crude) was obtained as a light-yellow oil. ¹H NMR: (400 MHz, CDCl₃) δ 5.50-5.15 (m, 1H), 4.56-4.18 (m, 2H), 3.76-3.68 (m, 3H), 2.28-2.09 (m, 2H), 1.46-1.38 (m, 9H), 0.87 (d, J=3.0 Hz, 9H), 0.10-0.04 (m, 6H).

### Step 4: Synthesis of 1-(tert-butyl) 2-methyl (2S,4R)-5-acetoxy-4-((tert-butyldimethylsilyl)oxy)pyrrolidine-1,2-dicarboxylate

To a solution of 1-(tert-butyl) 2-methyl (2S,4R)-4-((tert-butyldimethylsilyl)oxy)-5-hydroxypyrrolidine-1,2-dicarboxylate (384 g, 1.02 mol, 1.00 eq), Ac₂O (156 g, 1.53 mol, 143 mL, 1.50 eq) and TEA (156 g, 1.54 mol, 215 mL, 1.51 eq) in DCM (2.00 L) was added DMAP (25.0 g, 204 mmol, 0.20 eq) at 25° C., then stirred at 25° C. for 2 h. TLC (petroleum ether/ethyl acetate=4/1) showed 1-(tert-butyl) 2-methyl (2S,4R)-4-((tert-butyldimethylsilyl)oxy)-5-hydroxypyrrolidine-1,2-dicarboxylate (R_{f}=0.40, 0.45) was consumed and the desired spot (R_{f}=0.45, 0.55) was detected. The combined reaction mixture was poured into saturate NaHCO₃ (2000 mL) and extracted with DCM (1000 mL*2). The combined organic phase was concentrated in vacuum. The residue was purified by silica gel chromatography (Petroleum ether/Ethyl acetate=10/1, R_{f}=0.45). 1-(tert-butyl) 2-methyl (2S,4R)-5-acetoxy-4-((tert-butyldimethylsilyl)oxy)pyrrolidine-1,2-dicarboxylate (220 g, 527 mmol, 51.5% yield, 100% purity) was obtained as a light-yellow oil. ¹H NMR: EW28523-2-P1A1 (400 MHz, CDCl₃) δ 6.17 (s, 1H), 4.56-4.37 (m, 1H), 4.14 (d, J=7.2 Hz, 1H), 3.77 (s, 3H), 2.18-2.07 (m, 5H), 1.45 (d, J=6.8 Hz, 9H), 0.87 (s, 9H), 0.16-0.07 (m, 6H).

### Step 5: Synthesis of 1-(tert-butyl) 2-methyl (2S,4R)-5-allyl-4-((tert-butyldimethylsilyl)oxy)pyrrolidine-1,2-dicarboxylate

To a solution of 1-(tert-butyl) 2-methyl (2S,4R)-5-acetoxy-4-((tert-butyldimethylsilyl)oxy)pyrrolidine-1,2-dicarboxylate (100 g, 239 mmol, 1.00 eq) in DCM (1.00 L) was added BF₃·Et₂O (85.1 g, 599 mmol, 74.0 mL, 2.50 eq) and allyltrimethylsilane (123 g, 1.08 mol, 172 mL, 4.50 eq) at −70° C. under N₂. The reaction mixture was stirred at −70° C. for 3 h. LCMS (EW28523-3-P1A) showed 1-(tert-butyl) 2-methyl (2S,4R)-5-acetoxy-4-((tert-butyldimethylsilyl)oxy)pyrrolidine-1,2-dicarboxylate was consumed and the desired mass (RT=1.153 mins) was detected. The reaction was quenched by saturated NaHCO₃ (1000 mL), then extracted with DCM (1000 mL*2). The combined organic layers were dried over Na₂SO₄, filtered and concentrated under vacuum. 1-(tert-butyl) 2-methyl (2S,4R)-5-allyl-4-((tert-butyldimethylsilyl)oxy)pyrrolidine-1,2-dicarboxylate (200 g, crude) was obtained as a yellow oil. The crude product was used for the next step without purification. LCMS: product RT=1.153 mins, m/z=300.1 [M-Boc]⁺. ¹H NMR: (400 MHz, CDCl₃) δ 5.97-5.74 (m, 1H), 5.17-4.95 (m, 1H), 4.67-3.94 (m, 3H), 3.81-3.70 (m, 3H), 2.65-2.42 (m, 1H), 2.37-2.15 (m, 1H), 2.12-1.92 (m, 2H), 1.60-1.36 (m, 8H), 0.92-0.83 (m, 9H), 0.09-0.03 (m, 6H)

### Step 6: Synthesis of Methyl (2S,4R)-5-allyl-4-((tert-butyldimethylsilyl)oxy)pyrrolidine-2-carboxylate

To a solution of 1-(tert-butyl) 2-methyl (2S,4R)-5-allyl-4-((tert-butyldimethylsilyl)oxy)pyrrolidine-1,2-dicarboxylate (200 g, 500 mmol, Crude purity, 1.00 eq) in DCM (1.50 mL) was added TFA (571 g, 5.01 mol, 370 mL, 10.0 eq) at 0° C., then stirred at 25° C. for 16 h. TLC (petroleum ether/ethyl acetate=2/1) showed 1-(tert-butyl) 2-methyl (2S,4R)-5-allyl-4-((tert-butyldimethylsilyl)oxy)pyrrolidine-1,2-dicarboxylate (R_{f}=0.50, 0.55) was consumed and the desired spot (R_{f}=0.35) was detected. The resulting mixture was quenched by saturated NaHCO₃(3000 mL) then extracted with DCM (2000 mL*2). The combined organic layers were dried over Na₂SO₄, filtered and concentrated under vacuum. The residue was purified by fast silica gel chromatography (petroleum ether/ethyl acetate=10/1, R_{f}=0.35). methyl (2S,4R)-5-allyl-4-((tert-butyldimethylsilyl)oxy)pyrrolidine-2-carboxylate (135 g, crude) was obtained as a yellow oil. ¹H NMR: (400 MHz, CDCl₃) δ 5.89-5.76 (m, 1H), 5.19-5.03 (m, 3H), 4.30-4.21 (m, 1H), 4.07 (t, J=8.2 Hz, 1H), 3.73 (s, 3H), 3.18 (dt, J=3.4, 7.0 Hz, 1H), 2.35-2.27 (m, 2H), 2.21-2.11 (m, 2H), 2.08-1.99 (m, 2H), 0.91-0.89 (m, 9H), 0.08 (d, J=5.4 Hz, 6H).

### Step 7: Synthesis of Methyl (2S,4R,5S)-5-allyl-1-((S)-2-((tert-butoxycarbonyl)amino)pent-4-enoyl)-4-((tert-butyldimethylsilyl)oxy)pyrrolidine-2-carboxylate and methyl (2S,4R,5R)-5-allyl-1-((S)-2-((tert-butoxycarbonyl)amino)pent-4-enoyl)-4-((tert-butyldimethylsilyl)oxy)pyrrolidine-2-carboxylate

To a solution of methyl (2S,4R)-5-allyl-4-((tert-butyldimethylsilyl)oxy)pyrrolidine-2-carboxylate (135 g, 451 mmol, crude purity, 1.00 eq) and (S)-2-((tert-butoxycarbonyl)amino)pent-4-enoic acid (111 g, 518 mmol, 1.15 eq) in DMF (1.30 L) was added HATU (343 g, 902 mmol, 2.00 eq) and DIEA (291 g, 2.25 mol, 393 mL, 5.00 eq) at 0° C., then stirred at 25° C. for 16 h. TLC (petroleum ether/ethyl acetate=3/1) showed methyl (2S,4R)-5-allyl-4-((tert-butyldimethylsilyl)oxy)pyrrolidine-2-carboxylate (R_{f}=0.40, 0.45) was consumed and the desired spot (R_{f}=0.50) was detected. The reaction was poured into water (4.00 L), then extracted with ethyl acetate (2.00 L*2). The combined organic layers were dried over Na₂SO₄, filtered and concentrated under vacuum. The residue was purified by pre-HPLC (column: Phenomenex Luna C18 (250*80 mm*15 um); mobile phase: [water (0.1% TFA)-ACN]; B %: 80%-100%, 35 min]). The aqueous was concentrated under vacuum to give product. methyl (2S,4R,5S)-5-allyl-1-((S)-2-((tert-butoxycarbonyl)amino)pent-4-enoyl)-4-((tert-butyldimethylsilyl)oxy)pyrrolidine-2-carboxylate (84.0 g, 163 mmol, 32.2% yield over three steps, 96.5% purity) was obtained as a light-yellow oil. methyl (2S,4R,5R)-5-allyl-1-((S)-2-((tert-butoxycarbonyl)amino)pent-4-enoyl)-4-((tert-butyldimethylsilyl)oxy)pyrrolidine-2-carboxylate (24.0 g, 48.3 mmol, 10.7% yield over three steps) was obtained as a light-yellow oil. HPLC methyl (2S,4R,5S)-5-allyl-1-((S)-2-((tert-butoxycarbonyl)amino)pent-4-enoyl)-4-((tert-butyldimethylsilyl)oxy)pyrrolidine-2-carboxylate: product RT=4.394 mins, 96.5% purity under 220 nm. ¹H NMR methyl (2S,4R,5S)-5-allyl-1-((S)-2-((tert-butoxycarbonyl)amino)pent-4-enoyl)-4-((tert-butyldimethylsilyl)oxy)pyrrolidine-2-carboxylate: (400 MHz, CDCl₃) δ 5.98-5.76 (m, 2H), 5.28-5.05 (m, 5H), 4.63-4.48 (m, 2H), 4.43-4.31 (m, 1H), 4.14-4.01 (m, 1H), 3.74 (s, 3H), 2.71-2.59 (m, 1H), 2.48-2.35 (m, 2H), 2.31 (s, 1H), 2.24-2.14 (m, 1H), 2.08-2.00 (m, 1H), 1.48-1.40 (m, 9H), 0.94-0.88 (m, 9H), 0.09 (d, J=3.2 Hz, 6H).

### Step 8a: Synthesis of Methyl (1R,3S,6S,10aS,Z)-6-((tert-butoxycarbonyl)amino)-1-((tert-butyldimethylsilyl)oxy)-5-oxo-1,2,3,5,6,7,10,10a-octahydropyrrolo[1,2-a]azocine-3-carboxylate

To a solution of methyl (2S,4R,5S)-5-allyl-1-((S)-2-((tert-butoxycarbonyl)amino)pent-4-enoyl)-4-((tert-butyldimethylsilyl)oxy)pyrrolidine-2-carboxylate (30.0 g, 58.3 mmol, 96.5% purity, 1.00 eq) in DCM (1.50 L) was added Grubbs 1st (9.59 g, 11.7 mmol, 0.20 eq) at 25° C., then heated to reflux (55° C.) for 16 h. LCMS (EW28523-6-P1A) showed methyl (2S,4R,5S)-5-allyl-1-((S)-2-((tert-butoxycarbonyl)amino)pent-4-enoyl)-4-((tert-butyldimethylsilyl)oxy)pyrrolidine-2-carboxylate remained (RT=1.138 mins) and the desired mass (RT=1.065 mins) was detected. The mixture was concentrated in vacuo. The residue was purified by silica gel chromatography (petroleum ether/ethyl acetate=10/1, petroleum ether/ethyl acetate=3/1, R_{f}=0.40). methyl (1R,3S,6S,10aS,Z)-6-((tert-butoxycarbonyl)amino)-1-((tert-butyldimethylsilyl)oxy)-5-oxo-1,2,3,5,6,7,10,10a-octahydropyrrolo[1,2-a]azocine-3-carboxylate (23.0 g, crude) was obtained as a brown oil. LCMS: product RT=1.065 mins, m/z=469.1 (M+H)⁺

### Step 8b: Synthesis of Methyl (1R,3S,6S,10aR,Z)-6-((tert-butoxycarbonyl)amino)-1-((tert-butyldimethylsilyl)oxy)-5-oxo-1,2,3,5,6,7,10,10a-octahydropyrrolo[1,2-a]azocine-3-carboxylate

To a solution of methyl (2S,4R,5R)-5-allyl-1-((S)-2-((tert-butoxycarbonyl)amino)pent-4-enoyl)-4-((tert-butyldimethylsilyl)oxy)pyrrolidine-2-carboxylate (16.3 g, 32.8 mmol, 1.00 eq) in DCM (800 mL) was added Grubb's 1st (2.70 g, 3.28 mmol, 0.10 eq) at 25° C., then heated to reflux (60° C.) for 16 h. LCMS (EW21491-265-P1A) showed methyl (2S,4R,5R)-5-allyl-1-((S)-2-((tert-butoxycarbonyl)amino)pent-4-enoyl)-4-((tert-butyldimethylsilyl)oxy)pyrrolidine-2-carboxylate was consumed and the desired mass (RT=1.077 mins) was detected. The resulting mixture was concentrated under vacuum. The residue was purified by column chromatography (SiO₂, petroleum ether/ethyl acetate=10/1 to 5/1, R_{f}=0.50, Plate: petroleum ether/ethyl acetate=1/1). methyl (1R,3S,6S,10aR,Z)-6-((tert-butoxycarbonyl)amino)-1-((tert-butyldimethylsilyl)oxy)-5-oxo-1,2,3,5,6,7,10,10a-octahydropyrrolo[1,2-a]azocine-3-carboxylate (18.0 g, crude) was obtained as a brown oil. LCMS: product RT=1.077 mins, m/z=469.1 (M+H)⁺

### Step 9a: Synthesis of Methyl (1R,3S,6S,10aS,Z)-6-((tert-butoxycarbonyl)amino)-1-hydroxy-5-oxo-1,2,3,5,6,7,10,10a-octahydropyrrolo[1,2-a]azocine-3-carboxylate

To a solution of methyl (1R,3S,6S,10aS,Z)-6-((tert-butoxycarbonyl)amino)-1-((tert-butyldimethylsilyl)oxy)-5-oxo-1,2,3,5,6,7,10,10a-octahydropyrrolo[1,2-a]azocine-3-carboxylate (23.0 g, 49.1 mmol, CRUDE purity, 1.00 eq) in THF (220 mL) was added TBAF (1 M, 98.1 mL, 2.00 eq) at −10° C., then stirred at 0° C. for 1 hr. TLC (petroleum ether/ethyl acetate=3/1, Plate 1) showed methyl (1R,3S,6S,10aS,Z)-6-((tert-butoxycarbonyl)amino)-1-((tert-butyldimethylsilyl)oxy)-5-oxo-1,2,3,5,6,7,10,10a-octahydropyrrolo[1,2-a]azocine-3-carboxylate (R_{f}=0.30) was consumed and the desired spot (R_{f}=0.00) was detected. The reaction was poured into water (50.0 mL) then extracted with ethyl acetate (50.0 mL*2). The combined organic layers were dried over Na₂SO₄, filtered and concentrated under vacuum. The residue was triturated with petroleum ether/ethyl acetate=2/1 (80.0 mL) at 20° C. for 0.5 h, then filtered. The filter cake was dried under vacuum. methyl (1R,3S,6S,10aS,Z)-6-((tert-butoxycarbonyl)amino)-1-hydroxy-5-oxo-1,2,3,5,6,7,10,10a-octahydropyrrolo[1,2-a]azocine-3-carboxylate (5.20 g, 14.2 mmol, 12.1% yield over two step, 96.7% purity) was obtained as a light-yellow solid. LCMS: product RT=0.774 min, m/z=299.0 [M−55]⁺ HPLC product RT=1.910 mins, 96.7% purity under 220 nm SFC: RT=1.218 mins, ee %=100% (220 nm)¹H NMR: (400 MHz, CDCl₃) δ 6.05-5.71 (m, 3H), 4.77-4.62 (m, 2H), 4.41 (d, J=3.2 Hz, 1H), 4.30-4.22 (m, 1H), 3.75 (s, 3H), 2.89-2.78 (m, 1H), 2.71-2.55 (m, 2H), 2.46 (ddd, J=3.0, 8.2, 16.8 Hz, 1H), 2.26 (ddd, J=3.8, 9.2, 13.4 Hz, 1H), 2.16-1.95 (m, 2H), 1.44 (s, 9H)

### Step 9b: Synthesis of Methyl (1R,3S,6S,10aR,Z)-6-((tert-butoxycarbonyl)amino)-1-hydroxy-5-oxo-1,2,3,5,6,7,10,10a-octahydropyrrolo[1,2-a]azocine-3-carboxylate

To a solution of methyl (1R,3S,6S,10aR,Z)-6-((tert-butoxycarbonyl)amino)-1-((tert-butyldimethylsilyl)oxy)-5-oxo-1,2,3,5,6,7,10,10a-octahydropyrrolo[1,2-a]azocine-3-carboxylate (18.0 g, 38.4 mmol, CRUDE purity, 1.00 eq) in THF (100 mL) was added TBAF (1 M, 76.8 mL, 2.00 eq) at −10° C., then stirred at 0° C. for 1 hr. TLC (petroleum ether/ethyl acetate=3/1, Plate 1) showed methyl (1R,3S,6S,10aR,Z)-6-((tert-butoxycarbonyl)amino)-1-((tert-butyldimethylsilyl)oxy)-5-oxo-1,2,3,5,6,7,10,10a-octahydropyrrolo[1,2-a]azocine-3-carboxylate (R_{f}=0.30) was consumed and the desired spot (R_{f}=0.00) was detected. The reaction was poured into water (200 mL) then extracted with ethyl acetate (100 mL*2). The combined organic layers were dried over Na₂SO₄, filtered and concentrated under vacuum. The residue was purified by prep-HPLC (TFA condition), then adjusted pH about 7 with saturated NaHCO₃ aqueous (50.0 mL), extracted with ethyl acetate (500 mL*2). The combined organic layers were dried over Na₂SO₄, filtered and concentrated under vacuum. methyl (1R,3S,6S,10aR,Z)-6-((tert-butoxycarbonyl)amino)-1-hydroxy-5-oxo-1,2,3,5,6,7,10,10a-octahydropyrrolo[1,2-a]azocine-3-carboxylate (4.20 g, 11.4 mmol, 36.0% yield over two step, 96.2% purity) was obtained as a yellow solid LCMS: product RT=0.776 min, m/z=299.0 (M−55)⁺ HPLC: product RT=1.960 mins, 96.2% purity under 220 nm SFC: RT=1.483 mins, ee %=100% (220 nm)¹H NMR: (400 MHz, CDCl₃) δ 5.96-5.81 (m, 1H), 5.78-5.63 (m, 2H), 4.84-4.65 (m, 1H), 4.58 (t, J=7.2 Hz, 1H), 4.16 (d, J=4.6 Hz, 1H), 3.89 (d, J=4.6 Hz, 1H), 3.72 (s, 3H), 3.40-2.82 (m, 1H), 2.65 (t, J=16.8 Hz, 2H), 2.55-2.34 (m, 2H), 2.27-2.07 (m, 2H), 1.42 (s, 9H).

### Synthesis of (3S,6S,9R,10aR)-6-((tert-butoxycarbonyl)amino)-9-ethyl-5-oxodecahydropyrrolo[1,2-a]azocine-3-carboxylic acid and (3S,6S,9S,10aR)-6-((tert-butoxycarbonyl)amino)-9-ethyl-5-oxodecahydropyrrolo[1,2-a]azocine-3-carboxylic acid

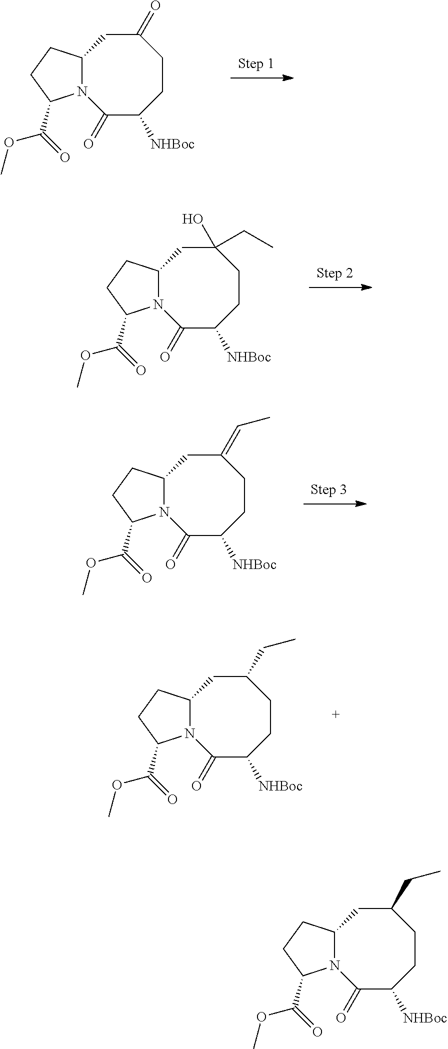

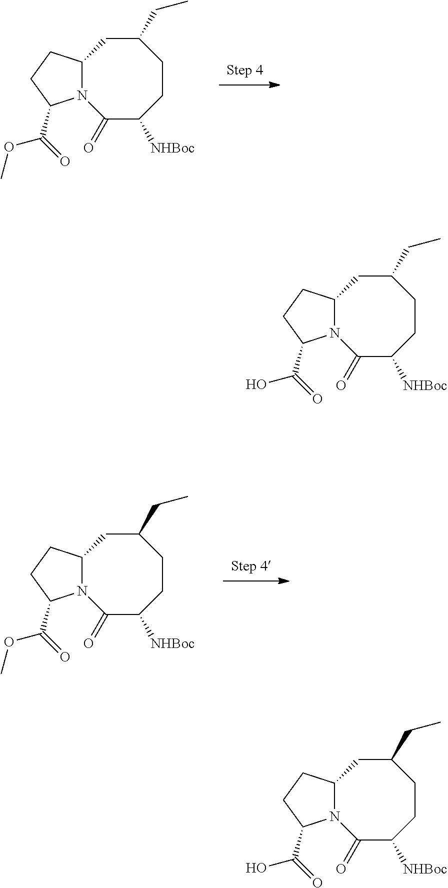 [SMILES: CC[C@@H]1CC[C@H](NC(=O)OC(C)(C)C)C(=O)N2[C@H](CC[C@H]2C(=O)OC)C1 ; CC[C@@H]1CC[C@H](NC(=O)OC(C)(C)C)C(=O)N2[C@H](CC[C@H]2C(=O)O)C1 ; CC[C@H]1CC[C@H](NC(=O)OC(C)(C)C)C(=O)N2[C@H](CC[C@H]2C(=O)OC)C1 ; CC[C@H]1CC[C@H](NC(=O)OC(C)(C)C)C(=O)N2[C@H](CC[C@H]2C(=O)O)C1]

### Step 1: Preparation of Methyl (3S,6S,10aR)-6-((tert-butoxycarbonyl)amino)-9-ethyl-9-hydroxy-5-oxodecahydropyrrolo[1,2-a]azocine-3-carboxylate

To a solution of methyl (3S,6S,10aR)-6-((tert-butoxycarbonyl)amino)-5,9-dioxodecahydropyrrolo[1,2-a]azocine-3-carboxylate (250 mg, 705 μmol, 1 eq) in anhydrous tetrahydrofuran (10 mL) was added ethyl magnesium chloride solution (2 M in THF) (720 μL, 1.44 mmol, 2.05 eq) at 0° C. The reaction mixture was stirred at 0° C. for 2 h, then quenched with saturated aq. NH₄Cl. The mixture was extracted with EtOAc (2×15 mL) and the combined organic layers were dried with Na₂SO₄, filtered, and concentrated under reduced pressure. The crude product was purified by reverse phase chromatography on a 30 g C₁₈ cartridge eluting with 5-60% MeCN in water (with 0.1% formic acid). The combined fractions were concentrated under reduced pressure and then freeze-dried to give methyl (3S,6S,10aR)-6-((tert-butoxycarbonyl)amino)-9-ethyl-9-hydroxy-5-oxodecahydropyrrolo[1,2-a]azocine-3-carboxylate (197 mg, 72.6%) as a white solid. LCMS (ESI): m/z=385.2 [M+H]⁺. ¹H NMR (400 MHz, CDCl₃) δ 5.38 (d, J=7.8 Hz, 1H), 5.08-4.96 (m, 1H), 4.58 (dd, J=11.7, 7.6 Hz, 1H), 4.53-4.43 (m, 1H), 3.78-3.71 (m, 3H), 2.41-2.29 (m, 1H), 2.26-2.12 (m, 3H), 2.06-1.91 (m, 2H), 1.73 (dd, J=12.1, 6.7 Hz, 1H), 1.68-1.57 (m, 2H), 1.54-1.46 (m, 4H), 1.44-1.38 (m, 9H), 0.91 (t, J=7.5 Hz, 3H).

The intermediates shown in Table 40 were prepared according to the protocol described above in Step 1 with appropriate modifications.

TABLE 40

| Name | Structure | LCMS | NMR |
|---|---|---|---|
| methyl (3S,6S,10aR)-6- ((tert-butoxy-carbonyl) amino)-9-hydroxy-9- isopropyl-5- oxodecahydropyrrolo [1,2-a]azocine-3- carboxylate | 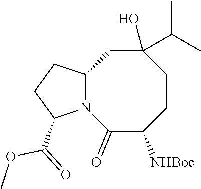 | 399.4 [M + H]⁺ | ¹H NMR (400 MHZ, CDCl₃) δ 5.40 (d, J = 7.8 Hz, 1H), 5.08- 4.95 (m, 1H), 4.59 (dd, J = 11.1, 8.2 Hz, 1H), 4.53-4.42 (m, 1H), 3.76 (s, 3H), 2.39-2.29 (m, 1H), 2.25-2.10 (m, 3H), 2.08-1.94 (m, 2H), 1.78-1.47 (m, 6H), 1.43 (s, 9H), 0.91 (dd, J = 6.7, 3.3 Hz, 6H) |

### Step 2: Preparation of Methyl (3S,6S,10aR,E)-6-((tert-butoxycarbonyl)amino)-9-ethylidene-5-oxodecahydropyrrolo[1,2-a]azocine-3-carboxylate

To a solution of methyl (3S,6S,10aR)-6-((tert-butoxycarbonyl)amino)-9-ethyl-9-hydroxy-5-oxodecahydropyrrolo[1,2-a]azocine-3-carboxylate (197 mg, 512 μmol, 1 eq) in anhydrous toluene (5 mL) was added Burgess' reagent (486 mg, 2.04 mmol, 4 eq). The reaction mixture was warmed up to 70° C. and stirred for 1 h. Solvent was evaporated. The crude product was purified by reverse phase chromatography on a 50 g C₁₈ cartridge eluting with 5-100% MeCN in water (with 0.1% formic acid). The combined fractions were concentrated under reduced pressure to give methyl (3S,6S,10aR,E)-6-((tert-butoxycarbonyl)amino)-9-ethylidene-5-oxodecahydropyrrolo[1,2-a]azocine-3-carboxylate (158 mg, 84.4%) as a yellow oil. LCMS (ESI): m/z=367.4 [M+H]⁺.

The intermediates shown in Table 41 were prepared according to the protocol described above in Step 2 with appropriate modifications.

TABLE 41

| Name | Structure | LCMS |
|---|---|---|
| methyl (3S,6S,10aR)-6- ((tert-butoxycarbonyl)amino)- 5-oxo-9-(propan-2-ylidene) decahydropyrrolo[1,2-a] azocine-3-carboxylate | 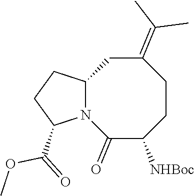 [SMILES: COC(=O)[C@@H]1CC[C@@H]2CC(=C(C)C)CC[C@H](NC(=O)OC(C)(C)C)C(=O)N21] | 381.2 [M + H]⁺ |

### Step 3: Preparation of Methyl (3S,6S,9R,10aR)-6-((tert-butoxycarbonyl)amino)-9-ethyl-5-oxodecahydropyrrolo[1,2-a]azocine-3-carboxylate and methyl (3S,6S,9S,10aR)-6-((tert-butoxycarbonyl)amino)-9-ethyl-5-oxodecahydropyrrolo[1,2-a]azocine-3-carboxylate

To a solution of methyl (3S,6S,10aR)-6-((tert-butoxycarbonyl)amino)-9-ethylidene-5-oxodecahydropyrrolo[1,2-a]azocine-3-carboxylate (158 mg, 431 μmol, 1 eq) in methanol (5 mL) under nitrogen bubbling was added 10% palladium on carbon (50% wet) (136 mg, 129 μmol, 0.3 eq). Hydrogen was bubbled for 5 min. into the suspension and then the reaction mixture was stirred at room temperature for 20 h under hydrogen atmosphere (1 atm). Nitrogen was bubbled into the suspension for 5 min. The reaction mixture was filtered over Celite (rinsing with MeOH) and the filtrate was concentrated under reduced pressure. The crude product was purified by reverse phase chromatography on a 100 g C₁₈ cartridge eluting with 5-100% MeCN in water (with 0.1% formic acid). The combined fractions were concentrated under reduced pressure and then freeze-dried to give methyl (3S,6S,9R,10aR)-6-((tert-butoxycarbonyl)amino)-9-ethyl-5-oxodecahydropyrrolo[1,2-a]azocine-3-carboxylate (86.0 mg, 54.4%, major isomer) as a white solid. LCMS (ESI): m/z=369.2 [M+H]⁺. ¹H NMR (400 MHz, C₆D₆) δ 5.69 (d, J=8.3 Hz, 1H), 4.91-4.84 (m, 1H), 4.45 (t, J d8.7 Hz, 1H), 3.66-3.62 (m, 1H), 3.37-3.29 (i, 3H), 2.18-2.15 (m, 1H), 1.96-1.68 (m, 1H), 1.72-1.65 (4, 1H), 1.65-1.50 (m, 3H), 1.44 (s, 9H), 1.33-1.30 (m, 1H), 1.25-1.14 (m, 2H), 1.11-1.02 (m, 2H), 1.00-0.90 (m, 2H), 0.74 (t, J=7.3 Hz, 3H). Methyl (3S,6S,9S,10aR)-6-((tert-butoxycarbonyl)amino)-9-ethyl-5-oxodecahydropyrrolo[1,2-a]azocine-3-carboxylate (10.0 mg, 6.32%, minor isomer) was also isolated as a white solid. LCMS (ESI): m/z 369.2 [M+H]⁺. ¹H NMR (400 MHz, C₆D₆) δ 6.20 (d, J=6.8 Hz, 1H), 4.57-4.45 (m, 1H), 4.36 (t, J=9.0 Hz, 1H), 3.62-3.50 (m, 1H), 3.31 (s, 3H), 2.27-2.22 (m, 1H), 1.77-1.65 (m, 2H), 1.63-1.47 (m, 3H), 1.45 (s, 9H), 1.37-1.29 (m, 1H), 1.25-1.12 (i, 3H), 1.11-1.01 (m, 1H), 0.95-10.88 (m, 1H), 0.83 (t, J 7.5 Hz, 3H), 0.75-0.64 (n, 1H). Note: Absolute stereochemistry on carbon bearing ethyl group is unknown for both isomers.

The intermediates shown in Table 42 were prepared according to the protocol described above in Step 3 with appropriate modifications. Note: Absolute stereochemistry on carbon bearing isopropyl group is unknown for both isomers.

TABLE 42

| Name | Structure | LCMS | NMR |
|---|---|---|---|
| methyl (3S,6S,9R,10aR)-6- ((tert- butoxycarbonyl)amino)-9- isopropyl-5- oxodecahydropyrrolo[1,2- a]azocine-3-carboxylate (Major Isomer) | 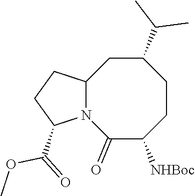 [SMILES: COC(=O)[C@@H]1CCC2C[C@H](C(C)C)CC[C@H](NC(=O)OC(C)(C)C)C(=O)N21] | 383.2 [M + H]⁺ | ¹H NMR (400 MHz, C₆D₆) δ 5.70 (d, J = 8.6 Hz, 1H), 4.95 − 4.82 (m, 1H), 4.46 (t, J = 8.8 Hz, 1H), 3.65 (dd, J = 11.6, 7.9 Hz, 1H), 3.35 −3.30 (m, 3H), 2.11 − 1.98 (m, 1H), 1.95 − 1.84 (m, 1H), 1.73 − 1.65 (m, 1H), 1.64 − 1.51 (m, 3H), 1.45 − 1.41 (m, 9H), 1.37 − 1.31 (m, 2H), 1.28 − 1.18 (m, 2H), 1.00 − 0.90 (m, 2H), 0.73 (t, J = 6.4 Hz, 6H) |
| methyl (3S,6S,9S,10aR)-6- ((tert- butoxycarbonyl)amino)-9- isopropyl-5- oxodecahydropyrrolo[1,2- a]azocine-3-carboxylate (Minor Isomer) | 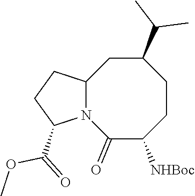 [SMILES: COC(=O)[C@@H]1CCC2C[C@@H](C(C)C)CC[C@H](NC(=O)OC(C)(C)C)C(=O)N21] | 383.2 [M + H]⁺ | ¹H NMR (400 MHz, C₆D₆) δ 6.25 (d, J = 6.8 Hz, 1H), 4.50 − 4.42 (m, 1H), 4.35 (t, J = 9.0 Hz, 1H), 3.54 − 3.49 (m, 1H), 2.30 − 2.21 (m, 1H), 1.76 − 1.66 (m, 2H), 1.61 − 1.47 (m, 3H), 1.46 − 1.42 (m, 10H), 1.42 − 1.27 (m, 4H), 1.14 − 1.02 (m, 2H), 0.95 − 0.89 (m, 1H), 0.81 − 0.77 (m, 7H) |

### Step 4: Preparation of (3S,6S,9R,10aR)-6-((tert-butoxycarbonyl)amino)-9-ethyl-5-oxodecahydropyrrolo[1,2-a]azocine-3-carboxylic acid

To a solution of methyl (3S,6S,9R,10aR)-6-((tert-butoxycarbonyl)amino)-9-ethyl-5-oxodecahydropyrrolo[1,2-a]azocine-3-carboxylate (40 mg, 108 μmol, 1 eq) in tetrahydrofuran (6 mL) and water (2 mL) was added lithium hydroxide monohydrate (13.5 mg, 324 μmol, 3 eq). The reaction mixture was stirred 4 h at room temperature. The reaction mixture was concentrated under reduced pressure (to remove THF). The crude product was purified by reverse phase chromatography on a 50 g C₁₈ cartridge eluting with 5-60% MeCN in water (with 0.1% formic acid). The combined fractions were concentrated under reduced pressure and then freeze-dried to give (3S,6S,9R,10aR)-6-((tert-butoxycarbonyl)amino)-9-ethyl-5-oxodecahydropyrrolo[1,2-a]azocine-3-carboxylic acid (32.0 mg, 83.7%) as a white solid. LCMS (ESI): m/z=355.2 [M+H]⁺.

The intermediates shown in Table 43 were prepared according to the protocol described above in Step 4 with appropriate modifications.

TABLE 43

| Name | Structure | LCMS | NMR |
|---|---|---|---|
| (3S,6S,9R,10aR)-6-((tert- butoxycarbonyl)amino)-9- isopropyl-5- oxodecahydropyrrolo[1,2- a]azocine-3-carboxylic acid | 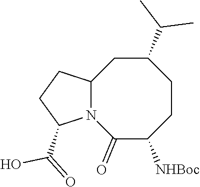 [SMILES: CC(C)[C@@H]1CC[C@H](NC(=O)OC(C)(C)C)C(=O)N2C(CC[C@H]2C(=O)O)C1] | 369.2 [M + H]⁺ | ¹H NMR (400 MHz, CDCl₃) δ 5.34 (d, J = 8.8 Hz, 1H), 4.81 − 4.70 (m, 1H), 4.62 (t, J = 8.1 Hz, 1H), 4.29 − 4.25 (m, 1H), 2.45 − 2.32 (m, 1H), 2.29 − 2.20 (m, 2H), 1.98 − 1.82 (m, 2H), 1.72 − 1.51 (m, 5H), 1.45 (s, 9H), 1.37 − 1.26 (m, 1H), 0.85 − 0.80 (m, 6H) |

### Step 4′: Preparation of (3S,6S,9S,10aR)-6-((tert-butoxycarbonyl)amino)-9-ethyl-5-oxodecahydropyrrolo[1,2-a]azocine-3-carboxylic acid

To a solution of methyl (3S,6S,9S,10aR)-6-((tert-butoxycarbonyl)amino)-9-ethyl-5-oxodecahydropyrrolo[1,2-a]azocine-3-carboxylate (10 mg, 27.1 μmol, 1 eq) in tetrahydrofuran (1 mL) and water (0.2 mL) was added lithium hydroxide monohydrate (3.41 mg, 81.3 μmol, 3 eq). The reaction mixture was stirred for 18 h at room temperature. 1 N HCl (1 mL) was added. The reaction mixture was concentrated under reduced pressure to give crude (3S,6S,9S,10aR)-6-((tert-butoxycarbonyl)amino)-9-ethyl-5-oxodecahydropyrrolo[1,2-a]azocine-3-carboxylic acid (9.6 mg, 100%) as a white solid. LCMS (ESI): m/z=355.2 [M+H]⁺.

The intermediates shown in Table 44 were prepared according to the protocol described above in Step 4 and Step 4′ with appropriate modifications.

TABLE 44

| Name | Structure | LCMS |
|---|---|---|
| (3S,6S,9S,10aR)-6-((tert- butoxycarbonyl)amino)- 9-isopropyl-5- oxodecahydropyrrolo [1,2-a]azocine-3- carboxylic acid | 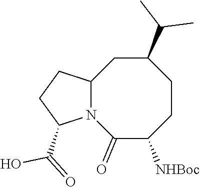 [SMILES: CC(C)[C@H]1CC[C@H](NC(=O)OC(C)(C)C)C(=O)N2C(CC[C@H]2C(=O)O)C1] | 369.2 [M + H]⁺ |

### Synthesis of (3S,6S,7S,10aS)-6-((tert-butoxycarbonyl)amino)-7-cyclopropyl-5-oxodecahydropyrrolo[1,2-a]azocine-3-carboxylic acid

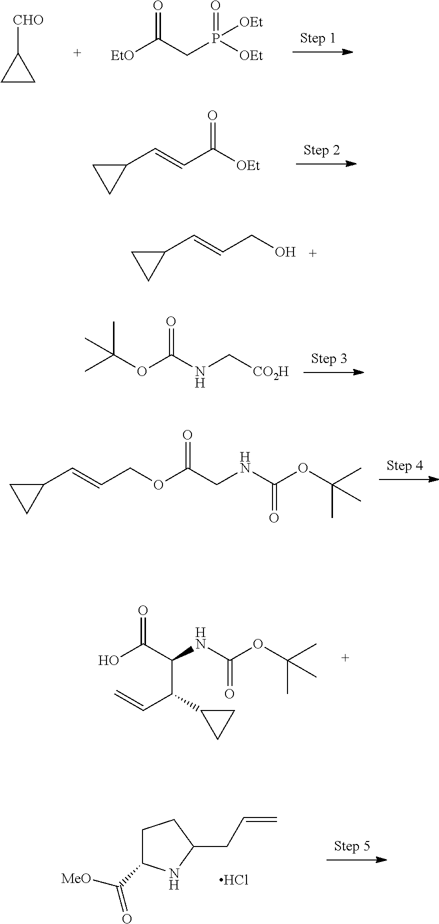

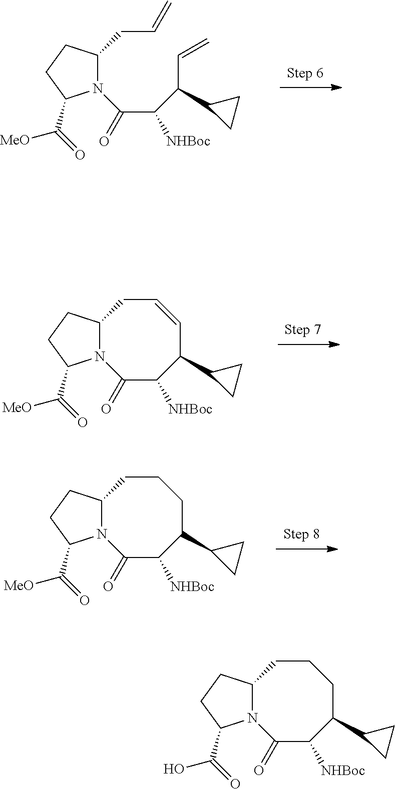

### Step 1: Preparation of Ethyl (E)-3-cyclopropylacrylate

To a suspension of 60% sodium hydride in mineral oil (658 mg, 16.5 mmol, 1.1 eq) in tetrahydrofuran (25 mL) at 0° C. was slowly added ethyl 2-(diethoxyphosphoryl)acetate (3.69 g, 16.5 mmol, 1.1 eq). The reaction was warmed up to room temperature and stirred for 30 min. A solution was observed. Cyclopropanecarbaldehyde (1.05 g, 15 mmol, 1 eq) was added dropwise at room temperature. An exotherm was observed and the formation of a thick paste. Stirring was difficult and the thick paste precipitated at the bottom of the flask. The reaction was stirred for 20 h at room temperature. The reaction was quenched with the addition of saturated NaHCO₃ aqueous solution (50 mL) and diluted with EtOAc (100 mL) and water (50 mL). The phases were separated and the aqueous layer was re-extracted with EtOAc (2×75 mL). The combined organic layers were dried with sodium sulfate, filtered, adsorbed on silica gel and carefully concentrated under reduced pressure (product is volatile and care should be taken during concentration in vacuo). The crude residue was purified by flash-chromatography on silica gel eluting with 1-25% DCM in pentane to give ethyl (E)-3-cyclopropylacrylate (1.25 g, 59.5%) as a clear liquid. ¹H NMR (400 MHz, CDCl₃) δ 6.44 (dd, J=15.3, 9.7 Hz, 1H), 5.91 (d, J=15.7 Hz, 1H), 4.15 (q, J=7.2 Hz, 2H), 1.64-1.53 (m, 1H), 1.29 (t, J=7.2 Hz, 3H), 0.99-0.93 (m, 2H), 0.69-0.62 (m, 2H).

### Step 2: Preparation of (E)-3-cyclopropylprop-2-en-1-ol

To a solution of ethyl (E)-3-cyclopropylacrylate (1.59 g, 11.3 mmol, 1 eq) in diethyl ether (50 mL) at −78° C. was slowly added 1 M DIBAL-H (24.0 mL, 24.0 mmol, 2.13 eq) in n-hexanes. The reaction was stirred for 1 h at −78° C. and 2.25 h at room temperature. The reaction was cooled down to 0° C. and quenched with the addition of MeOH (15 mL) and then 10% Rochelle salt aqueous solution (70 mL). The mixture was vigorously stirred for 1.5 h at room temperature and then the phases were separated. The organic layer was washed with brine, dried with sodium sulfate, filtered and concentrated under reduced pressure to give (E)-3-cyclopropylprop-2-en-1-ol (986 mg, 89.6%) as a clear liquid. ¹H NMR (400 MHz, CDCl₃) δ 5.75 (dt, J=15.2, 6.1 Hz, 1H), 5.29-5.21 (m, 1H), 4.09 (d, J=5.8 Hz, 2H), 1.48-1.38 (m, 1H), 0.78-0.72 (m, 2H), 0.43-0.38 (m, 2H).

### Step 3: Preparation of (E)-3-cyclopropylallyl (tert-butoxycarbonyl)glycinate

To solution of (E)-3-cyclopropylprop-2-en-1-ol (1.05 g, 10.6 mmol, 1 eq) and (tert-butoxycarbonyl)glycine (1.85 g, 10.6 mmol, 1 eq) in methylene chloride (50 mL) at 0° C. were added dicyclohexylcarbodiimide (2.18 g, 10.6 mmol, 1.0 eq) followed by DMAP (403 mg, 1.05 mmol, 0.1 eq). The reaction was warmed up to room temperature and stirred overnight.

The white precipitate was filtered off and the filtrate was washed with 1 N HCl (50 mL) and then saturated NaHCO₃ aqueous solution (50 mL). The organic layer was adsorbed on silica gel and concentrated under reduced pressure. The crude residue was purified by flash-chromatography on silica gel eluting with 0-50% EtOAc in heptane to give (E)-3-cyclopropylallyl (tert-butoxycarbonyl)glycinate (1.49 g, 55.1%) as a sticky off-white solid. ¹H NMR (400 MHz, CDCl₃) δ 5.66 (dt, J=15.4, 7.1 Hz, 1H), 5.34-5.29 (m, 1H), 5.05-4.97 (m, 1H), 4.61-4.57 (m, 2H), 3.98-3.82 (m, 2H), 1.47 (s, 9H), 1.44-1.39 (m, 1H), 0.80-0.74 (m, 2H), 0.46-0.40 (m, 2H).

### Step 4: Preparation of Racemic-(2S,3S)-2-((tert-butoxycarbonyl)amino)-3-cyclopropylpent-4-enoic acid

To a solution of (E)-3-cyclopropylallyl (tert-butoxycarbonyl)glycinate (1 g, 3.91 mmol, 1 eq) in tetrahydrofuran (40 mL) at −78° C. were added chlorotrimethylsilane (1.48 mL, 11.7 mmol, 3 eq) followed by the dropwise addition of a solution of 1 M LiHMDS (11.7 mL, 11.7 mmol, 3 eq) in THF. The reaction was kept in an ice-bath and stirred overnight while slowly warming up to room temperature. The reaction was quenched with 1 N HCl (3 mL) and concentrated under reduced pressure. The crude residue was purified by reverse phase chromatography on a C₁₈ cartridge eluting with 5-80% MeCN in water (with 0.1% formic acid) to give racemic-(2S,3S)-2-((tert-butoxycarbonyl)amino)-3-cyclopropylpent-4-enoic acid (730 mg, 73.1%) as a yellowish thick oil. ¹H NMR (400 MHz, CDCl₃) δ 5.83-5.68 (m, 1H), 5.28-5.10 (m, 3H), 4.51-4.43 (m, 1H), 1.93-1.82 (m, 1H), 1.45 (s, 9H), 0.94-0.82 (m, 1H), 0.62-0.49 (m, 2H), 0.34-0.24 (m, 1H), 0.19-0.08 (m, 1H).

### Step 5: Preparation of Methyl (2S,5R)-5-allyl-1-((2S,3S)-2-((tert-butoxycarbonyl)amino)-3-cyclopropylpent-4-enoyl)pyrrolidine-2-carboxylate

HATU (977 mg, 2.57 mmol, 1.2 eq) was added to a solution of crude methyl (2S)-5-allylpyrrolidine-2-carboxylate (434 mg, 2.57 mmol, eq) HCl salt, racemic-(2S,3S)-2-((tert-butoxycarbonyl)amino)-3-cyclopropylpent-4-enoic acid (550 mg, 2.15 mmol, eq) and DIPEA (2.23 mL, 12.9 mmol, 6 eq) in N,N-dimethylformamide (2 mL). The reaction was stirred at room temperature for 15 min. The product was purified directly by reverse phase chromatography on a C₁₈ cartridge eluting with 5-80% MeCN in water (with 0.1% formic acid) to give methyl (2S,5R)-5-allyl-1-((2S,3S)-2-((tert-butoxycarbonyl)amino)-3-cyclopropylpent-4-enoyl)pyrrolidine-2-carboxylate (525 mg, 60.0%) as a thick clear oil. LCMS (ESI) m/z=407.2 [M+H]⁺.

### Step 6: Preparation of Methyl (3S,6S,7R,10aR,Z)-6-((tert-butoxycarbonyl)amino)-7-cyclopropyl-5-oxo-1,2,3,5,6,7,10,10a-octahydropyrrolo[1,2-a]azocine-3-carboxylate

To a solution of methyl (2S,5R)-5-allyl-1-((2S,3S)-2-((tert-butoxycarbonyl)amino)-3-cyclopropylpent-4-enoyl)pyrrolidine-2-carboxylate (525 mg, 1.29 mmol, 1 eq) in methylene chloride (75 mL) under nitrogen was added Grubbs II catalyst (109 mg, 0.129 mmol, 0.1 eq). The reaction was heated to reflux for 48 h. The reaction was concentrated under reduced pressure. The crude residue was purified by reverse phase chromatography on a 100 g C₁₈ cartridge eluting with 5-80% MeCN in water (with 0.1% formic acid) to give methyl (3S,6S,7R,10aR,Z)-6-((tert-butoxycarbonyl)amino)-7-cyclopropyl-5-oxo-1,2,3,5,6,7,10,10a-octahydropyrrolo[1,2-a]azocine-3-carboxylate (218 mg, 44.6%) as a brownish oil (complex mixture of diastereomers). LCMS (ESI) m/z=379.2 [M+H]⁺.

### Step 7: Preparation of Methyl (3S,6S,7R,10aS)-6-((tert-butoxycarbonyl)amino)-7-cyclopropyl-5-oxodecahydropyrrolo[1,2-a]azocine-3-carboxylate

To a solution of methyl (3S,6S,7R,10aR,Z)-6-((tert-butoxycarbonyl)amino)-7-cyclopropyl-5-oxo-1,2,3,5,6,7,10,10a-octahydropyrrolo[1,2-a]azocine-3-carboxylate (218 mg, 0.5760 mmol, 1 eq) in ethanol (25 mL) under nitrogen was added 10% palladium on carbon (50% wet) (120 mg, 0.05638 mmol, 0.10 eq). Hydrogen was bubbled for 2 min. into the suspension and then the reaction was stirred at room temperature for 40 h under hydrogen atmosphere (1 atm). Nitrogen was bubbled into the suspension and the reaction was filtered over Celite (rinsed with MeOH). The filtrate was concentrated under reduced pressure. The crude residue was purified by reverse phase chromatography on a C₁₈ cartridge eluting with 5-80% MeCN in water (0.1% formic acid) to give methyl (3S,6S,7R,10aS)-6-((tert-butoxycarbonyl)amino)-7-cyclopropyl-5-oxodecahydropyrrolo[1,2-a]azocine-3-carboxylate (122 mg, 56%) as a white solid. LCMS (ESI) m/z=381.4 [M+H]⁺. ¹H NMR (400 MHz, C₆D₆) δ 5.56 (d, J=9.1 Hz, 1H), 4.95 (t, J=9.5 Hz, 1H), 4.39 (t, J=8.6 Hz, 1H), 3.74-3.65 (m, 1H), 3.36 (s, 3H), 2.39-2.29 (m, 1H), 1.80-1.70 (m, 1H), 1.68-1.50 (m, 5H), 1.46 (s, 9H), 1.20-1.06 (m, 1H), 1.05-0.78 (m, 4H), 0.74-0.61 (m, 1H), 0.27-0.16 (m, 2H), −0.23-−0.31 (m, 1H).

### Step 8: Preparation of (3S,6S,7S,10aS)-6-((tert-butoxycarbonyl)amino)-7-cyclopropyl-5-oxodecahydropyrrolo[1,2-a]azocine-3-carboxylic acid

To a solution of methyl (3S,6S,7R,10aS)-6-((tert-butoxycarbonyl)amino)-7-cyclopropyl-5-oxodecahydropyrrolo[1,2-a]azocine-3-carboxylate (122 mg, 0.3206 mmol, 1 eq) in tetrahydrofuran (3 mL) and water (1 mL) was added lithium hydroxide monohydrate (26.9 mg, 0.6412 mmol, 2 eq). The reaction was stirred for 20 h. The reaction was quenched with the addition of 1 N HCl (1 mL) and concentrated under reduced pressure to remove THF. The crude product was purified by reverse phase chromatography on a C₁₈ cartridge eluting with 5-80% MeCN in water (with 0.1% formic acid) to give (3S,6S,7S,10aS)-6-((tert-butoxycarbonyl)amino)-7-cyclopropyl-5-oxodecahydropyrrolo[1,2-a]azocine-3-carboxylic acid (100 mg, 85.4%) as a white solid. LCMS (ESI) m/z=367.1 [M+H]⁺. ¹H NMR (400 MHz, C₆D₆) δ 5.72-5.59 (m, 1H), 4.92-4.81 (m, 1H), 4.35-4.25 (m, 1H), 3.73-3.57 (m, 1H), 2.01-1.83 (m, 2H), 1.67-1.49 (m, 3H), 1.46 (s, 9H), 1.42-1.28 (m, 3H), 1.19-1.04 (m, 1H), 0.99-0.87 (m, 2H), 0.87-0.75 (m, 2H), 0.71-0.57 (m, 1H), 0.30-0.12 (m, 2H), −0.19-−0.31 (m, 1H).

### Synthesis of (3S,6S,9R,10aR)-6-((tert-butoxycarbonyl)amino)-5-oxo-9-propyldecahydropyrrolo[1,2-a]azocine-3-carboxylic acid and (3S,6S,9S,10aR)-6-((tert-butoxycarbonyl)amino)-5-oxo-9-propyldecahydropyrrolo[1,2-a]azocine-3-carboxylic acid

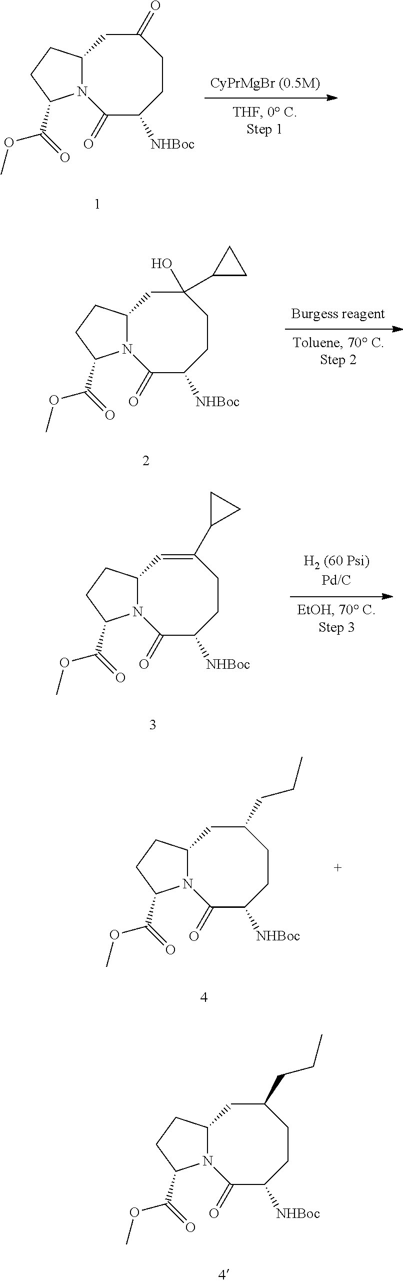

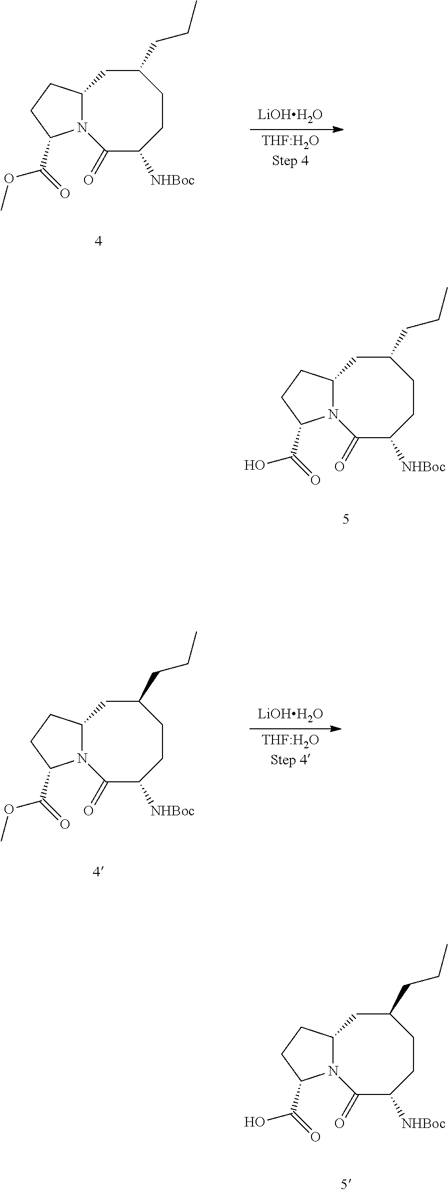

### Step 1: Preparation of Methyl (3S,6S,10aR)-6-((tert-butoxycarbonyl)amino)-9-cyclopropyl-9-hydroxy-5-oxodecahydropyrrolo[1,2-a]azocine-3-carboxylate

To a solution of methyl (3S,6S,10aR)-6-((tert-butoxycarbonyl)amino)-5,9-dioxodecahydropyrrolo[1,2-a]azocine-3-carboxylate (490 mg, 1.38 mmol, 1 eq) in anhydrous tetrahydrofuran (10 mL) was added cyclopropyl magnesium bromide solution (0.5 M in THF) (5.52 mL, 2.76 mmol, 2 eq) at 0° C. The reaction mixture was stirred at 0° C. for 2 h. Cyclopropyl magnesium bromide solution (0.5 M in THF) (5.52 mL, 2.76 mmol, 2 eq) was added. The reaction mixture was stirred at 0° C. for 2 h, then quenched with saturated aq. NH₄Cl. The product was extracted with EtOAc (2×15 mL) and the combined organic layers were dried with Na₂SO₄, filtered and concentrated under reduced pressure. The crude product was purified by reverse phase chromatography on a 50 g C₁₈ cartridge eluting with 5-60% MeCN in water (with 0.1% formic acid). The combined fractions were concentrated under reduced pressure and then freeze-dried to give methyl (3S,6S,10aR)-6-((tert-butoxycarbonyl)amino)-9-cyclopropyl-9-hydroxy-5-oxodecahydropyrrolo[1,2-a]azocine-3-carboxylate (420 mg, 76.7%) as a beige solid. LCMS (ESI): m/z=397.2 [M+H]⁺.

### Step 2: Preparation of Methyl (3S,6S,10aR)-6-((tert-butoxycarbonyl)amino)-9-cyclopropyl-5-oxo-1,2,3,5,6,7,8,10a-octahydropyrrolo[1,2-a]azocine-3-carboxylate

To a solution of methyl (3S,6S,10aR)-6-((tert-butoxycarbonyl)amino)-9-cyclopropyl-9-hydroxy-5-oxodecahydropyrrolo[1,2-a]azocine-3-carboxylate (420 mg, 1.05 mmol, 1 eq) in anhydrous toluene (15 mL) was added Burgess' reagent (1 g, 4.20 mmol, 4 eq). The reaction mixture was warmed up to 70° C. and stirred for 1 h. Solvent was evaporated. The crude product was purified by reverse phase chromatography on a 100 g Cis cartridge eluting with 5-100% MeCN in water (with 0.1% formic acid). The combined fractions were concentrated under reduced pressure to give methyl (3S,6S,10aR)-6-((tert-butoxycarbonyl)amino)-9-cyclopropyl-5-oxo-1,2,3,5,6,7,8,10a-octahydropyrrolo[1,2-a]azocine-3-carboxylate (285 mg, 71.7%) as a beige solid. LCMS (ESI): m/z=379.2 [M+H]⁺.

### Step 3: Preparation of Methyl (3S,6S,9R,10aR)-6-((tert-butoxycarbonyl)amino)-5-oxo-9-propyldecahydropyrrolo[1,2-a]azocine-3-carboxylate and methyl (3S,6S,9S,10aR)-6-((tert-butoxycarbonyl)amino)-5-oxo-9-propyldecahydropyrrolo[1,2-a]azocine-3-carboxylate

To a solution of methyl (3S,6S,10aR)-6-((tert-butoxycarbonyl)amino)-9-cyclopropyl-5-oxo-1,2,3,5,6,7,8,10a-octahydropyrrolo[1,2-a]azocine-3-carboxylate (190 mg, 502 μmol, 1 eq) in ethanol (10 mL) under nitrogen bubbling was added 10% palladium on carbon (50% wet) (159 mg, 150 μmol, eq). Hydrogen was bubbled into the suspension for 5 min. and then the reaction was stirred at 70° C. for 18 h under hydrogen pressure (60 psi). Nitrogen was bubbled for 5 min. into the suspension. The reaction mixture was filtered over Celite (rinsing with EtOH) and the filtrate was concentrated under reduced pressure. The crude product was purified by reverse phase chromatography on a 100 g C₁₈ cartridge eluting with 5-100% MeCN in water (with 0.1% formic acid). The combined fractions were concentrated under reduced pressure and then freeze-dried to give methyl (3S,6S,9R,10aR)-6-((tert-butoxycarbonyl)amino)-5-oxo-9-propyldecahydropyrrolo[1,2-a]azocine-3-carboxylate (107 mg, 55.7%, major isomer) as a white solid. LCMS (ESI): m/z=383.2 [M+H]⁺. ¹H NMR (400 MHz, C₆D₆) δ 5.71 (d, J=8.3 Hz, 1H), 4.94-4.82 (m, 1H), 4.45 (t, J=8.7 Hz, 1H), 3.69-3.60 (m, 1H), 3.34 (s, 3H), 2.22-2.11 (m, 1H), 2.02-1.91 (m, 1H), 1.71-1.52 (m, 3H), 1.44-1.42 (m, 9H), 1.43 (s, 9H), 1.35-1.25 (m, 2H), 1.22-1.16 (m, 1H), 1.15-1.05 (m, 2H), 1.04-0.89 (m, 5H), 0.83 (t, J=7.1 Hz, 3H). Methyl (3S,6S,9S,10aR)-6-((tert-butoxycarbonyl)amino)-5-oxo-9-propyldecahydropyrrolo[1,2-a]azocine-3-carboxylate (16.0 mg, 8.33%, minor isomer) was also isolated as a white solid. LCMS (ESI): m/z=383.2 [M+H]⁺. ¹H NMR (400 MHz, C₆D₆) δ 6.21 (d, J=7.1 Hz, 1H), 4.57-4.46 (m, 1H), 4.36 (t, J=9.0 Hz, 1H), 3.63-3.53 (m, 1H), 3.31 (s, 3H), 2.30-2.21 (m, 1H), 1.85-1.75 (m, 1H), 1.74-1.64 (m, 1H), 1.62-1.53 (m, 2H), 1.52-1.47 (m, 1H), 1.45 (s, 9H), 1.29-1.19 (m, 3H), 1.13-1.05 (m, 3H), 0.95-0.85 (m, 5H), 0.71-0.64 (m, 1H). Note: Absolute stereochemistry on carbon bearing n-propyl group is unknown for both isomers

### Step 4: Preparation of (3S,6S,9R,10aR)-6-((tert-butoxycarbonyl)amino)-5-oxo-9-propyldecahydropyrrolo[1,2-a]azocine-3-carboxylic acid

To a solution of methyl (3S,6S,9R,10aR)-6-((tert-butoxycarbonyl)amino)-5-oxo-9-propyldecahydropyrrolo[1,2-a]azocine-3-carboxylate (90 mg, 235 μmol, 1 eq) in tetrahydrofuran (6 mL) and water (2 mL) was added lithium hydroxide monohydrate (17 mg, 405 μmol, 1.7 eq). The reaction mixture was stirred overnight at room temperature. 1 N HCl (1 mL) was added. The reaction was concentrated under reduced pressure (to remove THF). The crude product was purified by reverse phase chromatography on a 30 g C₁₈ cartridge eluting with 5-100% MeCN in water (with 0.1% formic acid). The pure fractions were evaporated and lyophilized to give (3S,6S,9R,10aR)-6-((tert-butoxycarbonyl)amino)-5-oxo-9-propyldecahydropyrrolo[1,2-a]azocine-3-carboxylic acid (65.0 mg, 75.1%) as a white solid. LCMS (ESI): m/z=369.2 [M+H]⁺. ¹H NMR (400 MHz, DMSO-d₆) δ 6.79 (d, J=7.6 Hz, 1H), 4.55-4.28 (m, 1H), 4.25-4.15 (m, 2H), 2.25-1.99 (m, 2H), 1.94-1.78 (m, 2H), 2.28-1.78 (m, 4H), 1.76-1.44 (m, 6H), 1.40-1.28 (m, 11H), 1.22-1.14 (m, 2H), 0.86 (t, J=7.2 Hz, 3H).

### Step 4′: Preparation of (3S,6S,9S,10aR)-6-((tert-butoxycarbonyl)amino)-5-oxo-9-propyldecahydropyrrolo[1,2-a]azocine-3-carboxylic acid

To a solution of methyl (3S,6S,9S,10aR)-6-((tert-butoxycarbonyl)amino)-5-oxo-9-propyldecahydropyrrolo[1,2-a]azocine-3-carboxylate (15 mg, 39.2 μmol, 1 eq) in tetrahydrofuran (1 mL) and water (0.2 mL) was added lithium hydroxide monohydrate (4.90 mg, 117 μmol, 3 eq). The reaction mixture was stirred for 18 h at room temperature. 1 N HCl (1 mL) was added. The reaction was concentrated under reduced pressure (to remove THF). The crude product was purified by reverse phase chromatography on a 50 g C₁₈ cartridge eluting with 5-60% MeCN in water (with 0.1% formic acid). The pure fractions were evaporated and lyophilized to give (3S,6S,9S,10aR)-6-((tert-butoxycarbonyl)amino)-5-oxo-9-propyldecahydropyrrolo[1,2-a]azocine-3-carboxylic acid (13.0 mg, 90.2%) as a beige solid. LCMS (ESI): m/z=369.2 [M+H]⁺.

### Synthesis of methyl (3S,6S,8S,9aR)-6-((tert-butoxycarbonyl)amino)-8-methyl-5-oxooctahydro-1H-pyrrolo[1,2-a]azepine-3-carboxylate and methyl (3S,6S,8R,9aR)-6-((tert-butoxycarbonyl)amino)-8-methyl-5-oxooctahydro-1H-pyrrolo[1,2-a]azepine-3-carboxylate

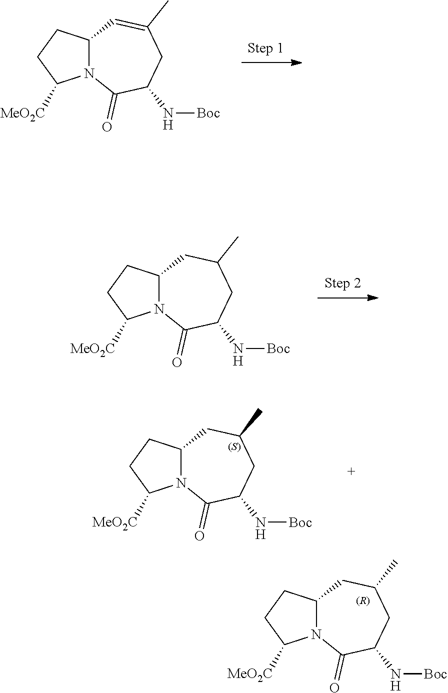

### Step 1: Preparation of Methyl (3S,6S,9aR)-6-((tert-butoxycarbonyl)amino)-8-methyl-5-oxooctahydro-1H-pyrrolo[1,2-a]azepine-3-carboxylate

To a solution of Pd/C (wet) (0.50 g, 4.85 mmol, 0.3 eq) in MeOH (50 mL) was added methyl (3S,6S,9aR)-6-((tert-butoxycarbonyl)amino)-8-methyl-5-oxo-2,3,5,6,7,9a-hexahydro-1H-pyrrolo[1,2-a]azepine-3-carboxylate (5.5 g, 16 mmol, 1 eq), the mixture was stirred at 25° C. for 2 hours under H₂ (15 Psi). The reaction mixture was filtered to give filtrate and concentrated under reduced pressure to give methyl (3S,6S,9aR)-6-((tert-butoxycarbonyl)amino)-8-methyl-5-oxooctahydro-1H-pyrrolo[1,2-a]azepine-3-carboxylate (5.70 g, crude) as a yellow oil. ¹H NMR (400 MHz, CD₃OD) δ 4.57-4.46 (m, 1H), 4.24-4.10 (m, 1H), 4.00-3.89 (m, 1H), 3.68 (d, J=1.2 Hz, 3H), 2.32-2.18 (m, 2H), 2.14-2.06 (m, 1H), 1.99-1.93 (m, 1H), 1.84-1.65 (m, 4H), 1.60-1.53 (m, 1H), 1.40 (s, 9H), 1.15 (d, J=7.2 Hz, 1H), 0.96 (d, J=6.4 Hz, 2H).

### Step 2: Preparation of Methyl (3S,6S,8S,9aR)-6-((tert-butoxycarbonyl)amino)-8-methyl-5-oxooctahydro-1H-pyrrolo[1,2-a]azepine-3-carboxylate and methyl (3S,6S,8R,9aR)-6-((tert-butoxycarbonyl)amino)-8-methyl-5-oxooctahydro-1H-pyrrolo[1,2-a]azepine-3-carboxylate

Methyl (3S,6S,9aR)-6-((tert-butoxycarbonyl)amino)-8-methyl-5-oxooctahydro-1H-pyrrolo[1,2-a]azepine-3-carboxylate (5.70 g, 16.70 mmol, 1 eq) was purified by SFC (Method\IC-3-MeOH (DEA)-5-40-3 mL-35 T·lcm) to give methyl (3S,6S,8S,9aR)-6-((tert-butoxycarbonyl)amino)-8-methyl-5-oxooctahydro-1H-pyrrolo[1,2-a]azepine-3-carboxylate (1.2 g, 3.5 mmol, 21% yield, Peak 2) as a light yellow oil. SFC (Rt=1.342 min). ¹H NMR (400 MHz, CD₃OD) δ 4.53 (d, J=4.0 Hz, 1H), 4.44 (d, J=3.2 Hz, 1H), 4.20-4.13 (m, 1H), 3.77-3.67 (m, 3H), 2.33-2.19 (m, 2H), 2.18-2.09 (m, 1H), 2.05-1.97 (m, 1H), 1.92-1.79 (m, 2H), 1.77-1.66 (m, 2H), 1.65-1.57 (m, 1H), 1.44 (s, 9H), 1.23-1.15 (m, 3H) and methyl (3S,6S,8R,9aR)-6-((tert-butoxycarbonyl)amino)-8-methyl-5-oxooctahydro-1H-pyrrolo[1,2-a]azepine-3-carboxylate (3.0 g, 8.8 mmol, 53% yield, Peak 1) as a light yellow oil. SFC (Rt=1.138 min). ¹H NMR (400 MHz, CD₃OD) δ 4.58 (d, J=3.6 Hz, 1H), 4.23 (d, J=11.2 Hz, 1H), 3.98 (d, J=10.4 Hz, 1H), 3.72 (s, 3H), 2.35-2.25 (m, 1H), 2.18-2.08 (m, 1H), 2.06-1.91 (m, 3H), 1.89-1.79 (m, 2H), 1.79-1.69 (m, 2H), 1.44 (s, 9H), 1.00 (d, J=6.4 Hz, 3H).

| Name | Structure | LCMS | NMR |
|---|---|---|---|
| methyl (3S,6S,8S,9aR)-6- ((tert- butoxycarbonyl)amino)-8- methyl-5-oxooctahydro-1H- pyrrolo[1,2-a]azepine-3- carboxylate* | 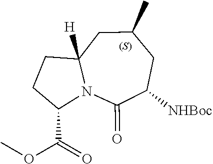 Peak 2 | | SFC (Rt = 1.342 min). ¹H NMR (400 MHz, CD₃OD) δ 4.53 (d, J = 4.0 Hz, 1H), 4.44 (d, J = 3.2 Hz, 1H), 4.20 − 4.13 (m, 1H), 3.77 − 3.67 (m, 3H), 2.33 − 2.19 (m, 2H), 2.18 − 2.09 (m, 1H), 2.05 − 1.97 (m, 1H), 1.92 − 1.79 (m, 2H), 1.77 − 1.66 (m, 2H), 1.65 − 1.57 (m, 1H), 1.44 (s, 9H), 1.23 − 1.15 (m, 3H) |
| | (stereochemistry confirmed via 2D NMR) | | |
| methyl (3S,6S,8R,9aR)-6- ((tert- butoxycarbonyl)amino)-8- methyl-5-oxooctahydro-1H- pyrrolo[1,2-a]azepine-3- carboxylate* | 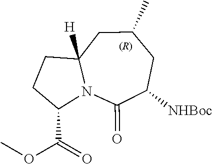 | | SFC (Rt = 1.138 min). ¹H NMR (400 MHz, CD₃OD) δ 4.58 (d, J = 3.6 Hz, 1H), 4.23 (d, J = 11.2 Hz, 1H), 3.98 (d, J = 10.4 Hz, 1H), 3.72 (s, 3H), 2.35 − 2.25 (m, 1H), 2.18 − 2.08 (m, 1H), 2.06 − 1.91 (m, 3H), 1.89 − 1.79 (m, 2H), 1.79 − 1.69 (m, 2H), 1.44 (s, 9H), 1.00 (d, J = 6.4 Hz, 3H) |
| | Peak 1 | | |
| | (stereochemistry confirmed via 2D NMR) | | |

*The individual isomers were separated by chiral SFC utilizing the following conditions:
Column: Chiralpak IC-3 50 × 4.6 mm, I.D. 3 um.
Mobile phase: CO2/MeOH/0.05% DEA).
Peak 1 r.t .= 1 minute,
Peak 2 r.t. = 1.135 minute.

### Procedures for Syntheses of Linkers

### Synthesis of 4-nitrophenyl 5-((bis(2-(benzoylthio)ethoxy)phosphoryl)difluoromethyl)benzo[b]thiophene-2-carboxylate

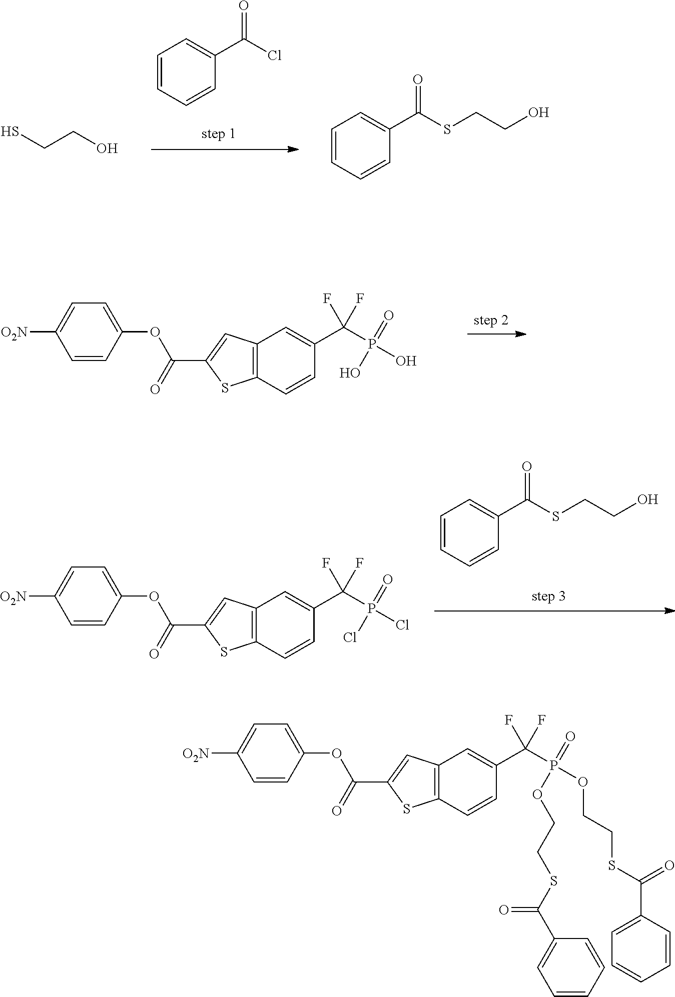

### Step 1: Preparation of S-(2-hydroxyethyl)benzothioate

To a solution of 2-mercaptoethanol (5.0 g, 64 mmol) and triethylamine (6.7 g, 64 mmol) in DCM (50 mL), then benzoyl chloride (8.1 g, 58 mmol) in DCM (10 mL) was added in it at −70° C. The mixture solution was stirred at 25° C. for 12 h to give a yellow solution. The reaction mixture was concentrated under reduced pressure to give a residue. The residue was purified by column chromatography to give S-(2-hydroxyethyl)benzothioate (5.60 g, 30.73 mmol, 48% yield) was obtained as a colorless oil. ¹H NMR (400 MHz, CDCl₃) δ 8.09-8.03 (m, 2H), 7.63-7.55 (m, 1H), 7.50-7.42 (m, 2H), 4.46 (t, J=6.4 Hz, 2H), 2.90 (td, J=6.8, 8.4 Hz, 2H)

The following intermediates in Table 45 were prepared using the described above in step 1 for the preparation of S-(2-hydroxyethyl)benzothioate and utilizing the appropriate starting materials and modifications.

TABLE 45

| Name | Structure | NMR |
|---|---|---|
| S-(2-hydroxyethyl) 2- phenylethanethioate | 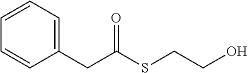 [SMILES: O=C(Cc1ccccc1)SCCO] | ¹H NMR (400 MHz, CDCl₃) δ 7.26 − 7.37 (m, 5H), 4.23 (t, J = 4.00 Hz, 2H), 3.66 (s, 2H), 2.73 (m, 2H). |

### Step 2: Preparation of 4-nitrophenyl 5-((dichlorophosphoryl)difluoromethyl)benzo[b]thiophene-2-carboxylate

To a solution of (difluoro(2-((4-nitrophenoxy)carbonyl)benzo[b]thiophen-5-yl)methyl)phosphonic acid (0.50 g, 1.2 mmol) and DMF (42 mg, 0.58 mmol) in DCM (5 mL) was added oxalic dichloride (0.44 g, 3.5 mmol) in DCM (5 mL) at 0° C., then the mixture solution was stirred at 40° C. for 1 h to give a yellow solution. The reaction mixture was concentrated under reduced pressure to give 4-nitrophenyl 5-((dichlorophosphoryl)difluoromethyl)benzo[b]thiophene-2-carboxylate (0.54 g, crude) as a yellow oil

### Step 3: Preparation of 4-nitrophenyl 5-((bis(2-(benzoylthio)ethoxy)phosphoryl)difluoromethyl)benzo[b]thiophene-2-carboxylate

To a solution of 4-nitrophenyl 5-((dichlorophosphoryl)difluoromethyl)benzo[b]thiophene-2-carboxylate (0.54 g, 1.1 mmol) in DCM (5 mL) was dropwise S-(2-hydroxyethyl)benzothioate (0.35 g, 1.9 mmol) at −78° C., then triethylamine (0.27 g, 2.7 mmol) in DCM (5 mL) was dropwise added in it at −78° C. and stirred at −78° C. for 5 min to give a yellow solution. The mixture was purified by column chromatography to give 4-nitrophenyl 5-((bis(2-(benzoylthio)ethoxy)phosphoryl)difluoromethyl)benzo[b]thiophene-2-carboxylate (0.20 g, 0.26 mmol, 25% yield) as a yellow oil. ¹H NMR (400 MHz, CDCl₃) δ 8.39-8.26 (m, 3H), 8.23-8.06 (m, 1H), 8.06-7.95 (m, 5H), 7.85-7.73 (m, 1H), 7.62-7.54 (m, 2H), 7.53-7.40 (m, 6H), 4.52-4.40 (m, 4H), 3.39-3.15 (m, 4H).

The following intermediates in Table 46 were prepared using the method described above in step 1 for the preparation of 4-nitrophenyl 5-((bis(2-(benzoylthio)ethoxy)phosphoryl)difluoromethyl)benzo[b]thiophene-2-carboxylate and utilizing the appropriate starting materials and modifications.

TABLE 46

| Name | Structure | LCMS |
|---|---|---|
| 4-nitrophenyl 5-((bis(2-((2- phenylacetyl)thio)ethoxy)phosphoryl) difluoromethyl)benzo[b]thiophene-2- carboxylate | 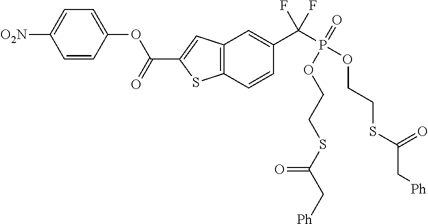 [SMILES: O=C(Cc1ccccc1)SCCOP(=O)(OCCSC(=O)Cc1ccccc1)C(F)(F)c1ccc2sc(C(=O)Oc3ccc([N+](=O)[O-])cc3)cc2c1] | 786.2 [M + H]⁺ |
| 4-nitrophenyl 5-((bis(2- (isobutyrylthio)ethoxy)phosphoryl) difluoromethyl)benzo[b]thiophene-2- carboxylate | 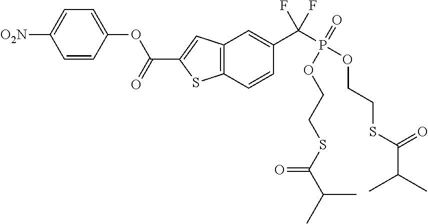 [SMILES: CC(C)C(=O)SCCOP(=O)(OCCSC(=O)C(C)C)C(F)(F)c1ccc2sc(C(=O)Oc3ccc([N+](=O)[O-])cc3)cc2c1] | 712.0 [M + Na]⁺ |

### Synthesis of 4-nitrophenyl 5-((bis(2-((2-methoxyacetyl)thio)ethoxy)phosphoryl)difluoromethyl)benzo[b]thiophene-2-carboxylate

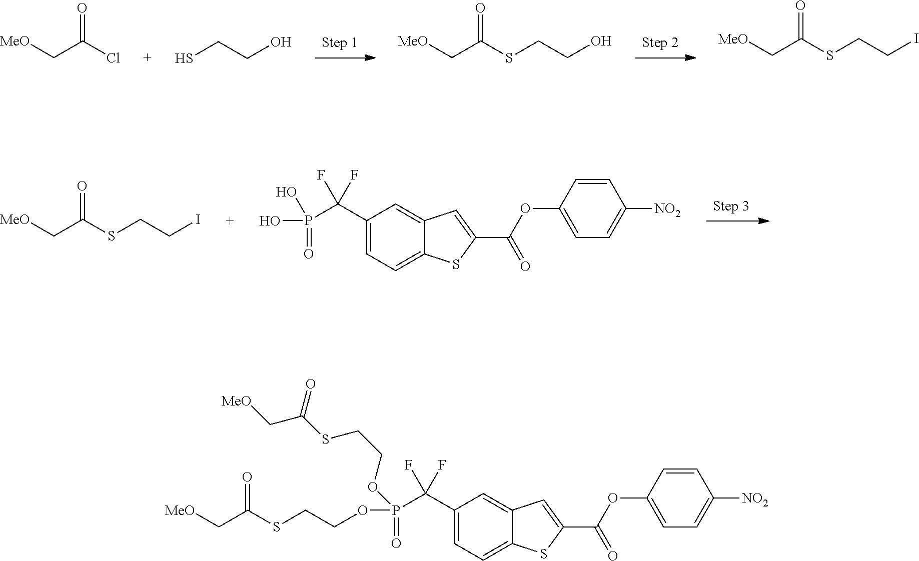

### Step 1: Preparation of S-(2-hydroxyethyl) 2-methoxyethanethioate

A solution of 2-methoxyacetyl chloride (1.5 g, 13.8 mmol, 1 eq) and triethylamine (1.91 mL, 13.8 mmol, 1.0 eq) in DCM (20 mL) was cooled down to −78° C. under nitrogen then 2-sulfanylethan-1-ol (1.07 g, 13.8 mmol, 1 eq) was added dropwise. The reaction was stirred for 1 h at −78° C. and then warmed up to room temperature and stirred for 1 h. The reaction was filtered and rinsed with DCM (20 mL) then the filtrate was washed with water (30 mL). The organic layer was washed with DCM (2×20 mL) and the combined organic extracts were adsorbed on silica gel and concentrated under reduced pressure. The residue was purified by flash-chromatography on silica gel eluting with 0-60% EtOAc in heptane to give S-(2-hydroxyethyl) 2-methoxyethanethioate (1.27 g, 61.3%) as a clear oil. ¹H NMR (400 MHz, CDCl₃) δ 4.11 (s, 2H), 3.78 (t, J=6.2 Hz, 2H), 3.49 (s, 3H), 3.12 (t, J=6.2 Hz, 2H).

### Step 2: Preparation of S-(2-iodoethyl) 2-methoxyethanethioate

To a solution of S-(2-hydroxyethyl) 2-methoxyethanethioate (500 mg, 3.32 mmol, 1 eq) in THF (10 mL) at 0° C. were added triphenylphosphine (870 mg, 3.32 mmol, 1.0 eq), imidazole (452 mg, 6.64 mmol, 2.0 eq) and iodine (842 mg, 3.32 mmol, 1.0 eq). The resulting brown solution was stirred for 1 h at 0° C. The solution was adsorbed on silica gel and concentrated under reduced pressure. The crude product was flash-chromatography on silica gel eluting with 0-30% EtOAc in heptane to give S-(2-iodoethyl) 2-methoxyethanethioate (757 mg, 87.7%) as clear oil. ¹H NMR (400 MHz, CDCl₃) δ 4.09 (s, 2H), 3.49 (s, 3H), 3.40-3.34 (m, 2H), 3.29-3.23 (m, 2H).

### Step 3: Preparation of 4-nitrophenyl 5-((bis(2-((2-methoxyacetyl)thio)ethoxy)phosphoryl)difluoromethyl)benzo[b]thiophene-2-carboxylate

Sodium hydroxide (74.3 mg, 1.86 mmol) in water (1 mL) was added dropwise to a stirred suspension of (difluoro(2-((4-nitrophenoxy)carbonyl)benzo[b]thiophen-5-yl)methyl)phosphonic acid (400 mg, 0.9317 mmol) in water (10 mL). When the mixture became pH˜8, a clear yellowish solution was obtained and silver nitrate (473 mg, 2.79 mmol) was added. A white precipitate was observed. After 2 h at room temperature, the white mixture was cooled down to 0° C. and the white precipitate was collected by filtration, washed with water and transferred with MeCN in a 100 mL flask. The solvent was removed under reduced pressure and the resulting beige solid was dried under vacuum. The powder was suspended in dry toluene (10 mL), and S-(2-iodoethyl) 2-methoxyethanethioate (787 mg, 3.02 mmol, 3.24 eq) was added. The mixture was stirred for 16 h at 50° C. The reaction was adsorbed on Celite and concentrated under reduced pressure. The crude residue was purified by flash-chromatography on silica gel eluting with a gradient of EtOAc in heptane (0 to 60%) to give 4-nitrophenyl 5-((bis(2-((2-methoxyacetyl)thio)ethoxy)phosphoryl)difluoromethyl)benzo[b]thiophene-2-carboxylate (178 mg, 27.5%) as a thick clear oil. ¹H NMR (400 MHz, CDCl₃) δ 8.39-8.34 (m, 3H), 8.23 (s, 1H), 8.02 (d, J=8.7 Hz, 1H), 7.76 (d, J=8.7 Hz, 1H), 7.48 (d, J=9.1 Hz, 2H), 4.35-4.17 (m, 4H), 4.09 (s, 4H), 3.49 (s, 6H), 3.26-3.11 (m, 4H).

### Synthesis of perfluorophenyl 5-((bis(2-((3-methoxypropanoyl)thio)ethoxy)phosphoryl)difluoromethyl)benzo[b]thiophene-2-carboxylate

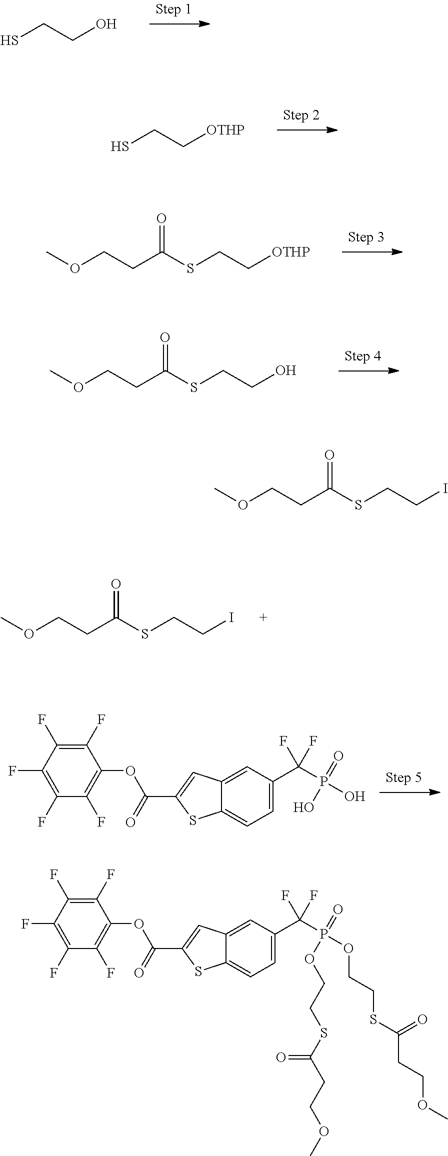

### Step 1: Preparation of 2-((tetrahydro-2H-pyran-2-yl)oxy)ethane-1-thiol

To a solution of 2-mercaptoethan-1-ol (9 g, 115 mmol, 1 eq.) and 3,4-dihydro-2H-pyran (14.5 g, 173 mmol, 1.5 eq.) in EtOH (200 mL) was added PPTS (2.9 g, 11.5 mmol, 0.1 eq). The reaction solution was stirred at 70° C. for 2 h under N₂. After completion, the reaction mixture was concentrated under reduced pressure. The residue was purified by flash column chromatography on silica gel to afford 2-((tetrahydro-2H-pyran-2-yl)oxy)ethane-1-thiol (12 g, 74.0 mmol, 64%) as a colorless oil. LCMS (ESI) m/z=163 [M+H]⁺.

### Step 2: Preparation of S-(2-((tetrahydro-2H-pyran-2-yl)oxy)ethyl) 3-methoxypropanethioate

To a solution of 3-methoxypropanoic acid (5.5 g, 52.8 mmol, 1 eq.) in DCM (30 mL) was added CDI (9.39 g, 58.0 mmol, 1.1 eq.) at 0° C. The solution was stirred at room temperature for 30 min, then 2-((tetrahydro-2H-pyran-2-yl)oxy)ethane-1-thiol (8.56 g, 52.8 mmol, 1 eq.) was added to the solution. The solution was stirred at room temperature for 2 h. After completion, the mixture was concentrated under reduced pressure. The residue was purified by flash column chromatography on silica gel to afford S-(2-((tetrahydro-2H-pyran-2-yl)oxy)ethyl) 3-methoxypropanethioate (8.00 g, 32.2 mmol, 61%) as a colorless oil. ¹H NMR (400 MHz, CDCl₃) δ 4.65-4.60 (m, 1H), 3.92-3.74 (m, 3H), 3.71-3.64 (m, 2H), 3.62-3.46 (m, 2H), 3.38-3.30 (m, 3H), 3.21-3.08 (m, 2H), 2.88-2.78 (m, 2H), 1.82 (ddd, J=15.1, 10.1, 3.4 Hz, 2H), 1.76-1.65 (m, 1H), 1.64-1.46 (m, 4H).

The following intermediates in Table 47 were prepared using the method described above in step 2 for the preparation of S-(2-((tetrahydro-2H-pyran-2-yl)oxy)ethyl) 3-methoxypropanethioate and utilizing the appropriate starting materials and modifications.

TABLE 47

| Name | Structure | LCMS |
|---|---|---|
| perfluorophenyl 5-((bis(2-((2-(2- methoxyethoxy)acetyl)thio)ethoxy) phosphoryl)difluoromethyl)benzo[b] thiophene-2-carboxylate | 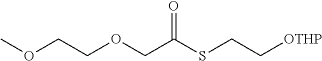 | 279 [M + H]⁺ |

### Step 3: Preparation of S-(2-hydroxyethyl) 3-methoxypropanethioate

To a solution of S-(2-((tetrahydro-2H-pyran-2-yl)oxy)ethyl) 3-methoxypropanethioate (8 g, 32.2 mmol, 1 eq.) in EtOH (40 mL) was added PPTS (809 mg, 3.22 mmol, 0.1 eq). The reaction solution was stirred at 50° C. for 2 h under N₂. After completion, the reaction mixture was concentrated under reduced pressure. The residue was purified by flash column chromatography on silica gel to afford S-(2-hydroxyethyl) 3-methoxypropanethioate (4.00 g, 24.3 mmol, 76%) as a colorless oil. LCMS (ESI) m/z=165 [M+H]⁺.

The following intermediates in Table 48 were prepared using the method described above in step 3 for the preparation of S-(2-hydroxyethyl) 3-methoxypropanethioate and utilizing the appropriate starting materials and modifications.

TABLE 48

| Name | Structure | LCMS |
|---|---|---|
| S-(2-hydroxyethyl) 2-(2- methoxyethoxy)ethanethioate | 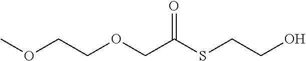 | 195 [M + H]⁺ |

### Step 4: Preparation of S-(2-iodoethyl) 3-methoxypropanethioate

To a solution of S-(2-hydroxyethyl) 3-methoxypropanethioate (1.4 g, 8.54 mmol, 1 eq.) and triphenylphosphine (2.2 g, 8.54 mmol, 1.0 eq.) in DCM (50 mL) was added 1-iodopyrrolidine-2,5-dione (1.92 g, 8.54 mmol, 1.0 eq.) at 0° C. The solution was stirred at room temperature for 1 hr. After completion, the reaction solution was concentrated under reduced pressure. The residue was purified by flash column chromatography on silica gel to afford S-(2-iodoethyl) 3-methoxypropanethioate (1.3 g, 4.74 mmol, 56%) as a yellow oil. LCMS (ESI) m/z=163 [M+H]⁺.

The following intermediates in Table 49 were prepared using the method described above in step 4 for the preparation of S-(2-iodoethyl) 3-methoxypropanethioate and utilizing the appropriate starting materials and modifications.

TABLE 49

| Name | Structure | LCMS |
|---|---|---|
| S-(2- iodoethyl) 2-(2- methoxy- | 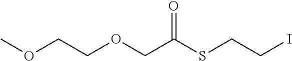 | 305 [M + H]⁺ |
| ethoxy) | | |
| ethanethioate | | |

### Step 5: Preparation of Perfluorophenyl 5-((bis(2-((3-methoxypropanoyl)thio)ethoxy)phosphoryl)difluoromethyl)benzo[b]thiophene-2-carboxylate

Amberlite IR120® (2.92 g, 127 mmol, 10 eq) in water (2 mL) was added dropwise to a stirred suspension of (difluoro(2-((perfluorophenoxy)carbonyl)benzo[b]thiophen-5-yl)methyl)phosphonic acid (6 g, 12.7 mmol, 1 eq.) in H₂O (60 mL). When the mixture became clear (pH˜9), silver(I) nitrate (4.75 g, 27.9 mmol, 2.2 eq.) was added. After stirring at 0° C. for 2 h, the gray precipitate was collected by filtration and dried under vacuum. To a solution of gray precipitate (2.2 g, 3.19 mmol, 1 eq.) in MeCN (10 mL) was added 1 S-(2-iodoethyl) 3-methoxypropanethioate (5, 2.00 g, 7.33 mmol, 2.3 eq). The solution was stirred at room temperature for 48 h. After completion, the mixture was filtered and the filtrate was purified by Prep-HPLC to afford perfluorophenyl 5-((bis(2-((3-methoxypropanoyl)thio)ethoxy)phosphoryl)difluoromethyl)benzo[b]thiophene-2-carboxylate (500 mg, 652 μmol, 20%) as a yellow oil. LCMS (ESI) m/z=767 [M+H]⁺.

The following intermediates in Table 50 were prepared using the method described above in step 5 for the preparation of perfluorophenyl 5-((bis(2-((3-methoxypropanoyl)thio)ethoxy)phosphoryl)difluoromethyl)benzo[b]thiophene-2-carboxylate and utilizing the appropriate starting materials and modifications.

TABLE 50

| Name | Structure | LCMS |
|---|---|---|
| perfluorophenyl 5-((bis(2-((2-(2- methoxyethoxy)acetyl)thio)ethoxy) phosphoryl)difluoromethyl) benzo[b]thiophene- 2-carboxylate | 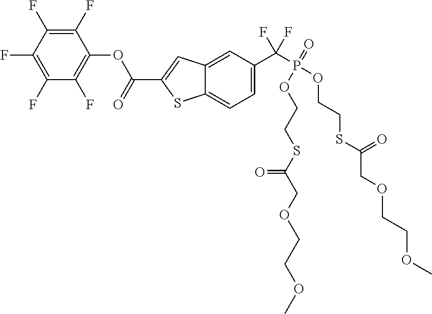 [SMILES: COCCOCC(=O)SCCOP(=O)(OCCSC(=O)COCCOC)C(F)(F)c1ccc2sc(C(=O)Oc3c(F)c(F)c(F)c(F)c3F)cc2c1] | |

### Synthesis of perfluorophenyl 7-((bis(2-((2-(oxetan-3-yl)acetyl)thio)ethoxy)phosphoryl)difluoromethyl)-2-naphthoate

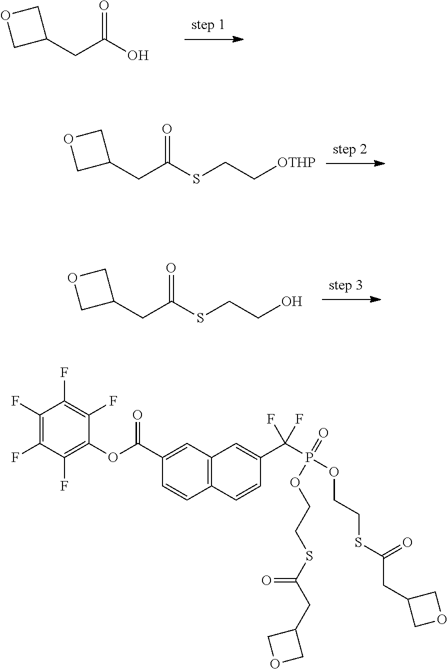

### Step 1: Preparation of S-(2-((tetrahydro-2H-pyran-2-yl)oxy)ethyl) 2-(oxetan-3-yl)ethanethioate

To a solution of 2-(oxetan-3-yl)acetic acid (1.0 g, 8.62 mmol, 1.0 eq.) and 2-((tetrahydro-2H-pyran-2-yl)oxy)ethane-1-thiol (1.5 g, 9.50 mmol, 1.1 eq.) in DCM (30 mL) was added CDI (2.79 g, 17.2 mmol, 2.0 eq.). The mixture was stirred at room temperature for 1 hr. After completion, the mixture was concentrated under reduced pressure. The residue was purified by flash column chromatography on silica gel to afford S-(2-((tetrahydro-2H-pyran-2-yl)oxy)ethyl) 2-(oxetan-3-yl)ethanethioate (1.1 g, 4.23 mmol, 49%) as a white solid. LCMS (ESI) m/z=261 [M+H]⁺.

### Step 2: Preparation of S-(2-hydroxyethyl) 2-(oxetan-3-yl)ethanethioate

To a solution of S-(2-((tetrahydro-2H-pyran-2-yl)oxy)ethyl) 2-(oxetan-3-yl)ethanethioate (1.1 g, 4.23 mmol, 1 eq.) in EtOH (15 mL) was added 4-methylbenzene-1-sulfonic acid; pyridine (106 mg) at room temperature. The reaction solution was stirred at 50° C. for 5 h under N₂. After completion, the reaction mixture was concentrated under reduced pressure. The residue was purified by flash column chromatography on silica gel to afford S-(2-hydroxyethyl) 2-(oxetan-3-yl)ethanethioate (600 mg, 3.40 mmol, 81%) as an oil. LCMS (ESI) m/z=177 [M+H]⁺.

### Step 3: Preparation of Perfluorophenyl 7-((bis(2-((2-(oxetan-3-yl)acetyl)thio)ethoxy)phosphoryl)difluoromethyl)-2-naphthoate

Oxalyl chloride (243 mg, 1.92 mmol, 3 eq.) was added dropwise to the solution of (difluoro(7-((perfluorophenoxy)carbonyl)naphthalen-2-yl)methyl)phosphonic acid (300 mg, 0.64 mmol, 1 eq.) in dry DCM (15 mL) and DMF (4.68 mg, 64 μmol, 0.1 eq.) at 20° C. The reaction mixture was stirred at 40° C. for an additional 1 hr. The reaction was monitored by pipetting out a small amount of crude sample and quenching it with MeOH to ensure bis-C₁ phosphoryl chloride had been formed completely (bis-methoxy phosphonate was observed by LCMS). After completion, the excess oxalyl chloride and solvent were removed under reduced pressure. The residue was re-dissolved in anhydrous DCM (2 mL), then added to a mixture of S-(2-hydroxyethyl) 2-(oxetan-3-yl)ethanethioate (3, 281 mg, 1.60 mmol, 2.5 eq.) and DIEA (330 mg, 2.56 mmol, 4 eq.) in anhydrous DCM (2 mL) at 0° C. The reaction was allowed to warm to 25° C. and stirred for an additional 2 h. After completion, the reaction mixture was quenched with HCl (0.3 mL, 2N) and concentrated under reduced pressure. The residue was purified by Biotage® C18 column chromatography to give to get perfluorophenyl 7-((bis(2-((2-(oxetan-3-yl)acetyl)thio)ethoxy)phosphoryl)difluoromethyl)-2-naphthoate (50.0 mg, 63 μmol, 10%). LCMS (ESI) m/z=785.0 [M+H]⁺.

### Synthesis of (E)-(((difluoro(4-(3-(4-nitrophenoxy)-3-oxoprop-1-en-1-yl)phenyl)methyl)phosphoryl)bis(oxy))bis(methylene)bis(2,2-dimethylpropanoate)

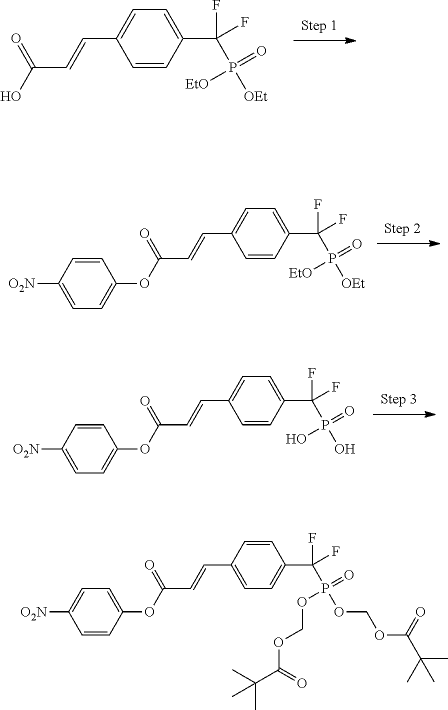 [SMILES: CCOP(=O)(OCC)C(F)(F)c1ccc(/C=C/C(=O)O)cc1 ; CCOP(=O)(OCC)C(F)(F)c1ccc(/C=C/C(=O)Oc2ccc([N+](=O)[O-])cc2)cc1 ; O=C(/C=C/c1ccc(C(F)(F)P(=O)(O)O)cc1)Oc1ccc([N+](=O)[O-])cc1 ; CC(C)(C)C(=O)OCOP(=O)(OCOC(=O)C(C)(C)C)C(F)(F)c1ccc(/C=C/C(=O)Oc2ccc([N+](=O)[O-])cc2)cc1]

### Step 1: Preparation of 4-nitrophenyl (E)-3-(4-((diethoxyphosphoryl)difluoromethyl)phenyl)acrylate

Oxalyl chloride (1.13 g, 8.98 mmol, 3 eq.) was added dropwise to the solution of (E)-3-(4-((diethoxyphosphoryl)difluoromethyl)phenyl)acrylic acid (1 g, 2.99 mmol, 1 eq.) in dry DCM (20 mL) and DMF (0.1 mL) at 0° C. The reaction mixture was stirred at 40° C. for an additional 1 hr. After completion, the excess oxalyl chloride and solvent were removed under reduced pressure. The residue was re-dissolved in anhydrous DCM (10 mL), then added to a mixture of 4-nitrophenol (457 mg, 3.29 mmol, 1.1 eq.) and TEA (1.5 g, 15.0 mmol, 5 eq.) in anhydrous DCM (10 mL) at 0° C. The reaction was allowed to warm to 25° C. and stirred for an additional 2 h. After completion, the reaction mixture was concentrated under reduced pressure. The residue was purified by flash column chromatography on silica gel to afford 4-nitrophenyl (E)-3-(4-((diethoxyphosphoryl)difluoromethyl)phenyl)acrylate (1 g, 2.20 mmol, 74%) as a white solid. LCMS (ESI): m/z=456 [M+H]⁺.

### Step 2: Preparation of (E)-(difluoro(4-(3-(4-nitrophenoxy)-3-oxoprop-1-en-1-yl)phenyl)methyl)phosphonic acid

To a solution of 4-nitrophenyl (E)-3-(4-((diethoxyphosphoryl)difluoromethyl)phenyl)acrylate (1 g, 2.20 mmol, 1.0 eq.) in DCM (15 mL) was added TMSBr (6.7 g, 44 mmol, 20.0 eq.) dropwise at 0° C. After the addition, the reaction mixture was allowed to warm to room temperature and stirred for an additional 12 h. The reaction progress was monitored by LCMS. After completion, the reaction was quenched by adding H₂O (5 mL) and extracted with DCM (10 mL×3). The organic layers were combined and washed with brine (20 mL), dried over with anhydrous Na₂SO₄, then concentrated under reduced pressure. The residue was purified by RP-prep-HPLC to afford (E)-(difluoro(4-(3-(4-nitrophenoxy)-3-oxoprop-1-en-1-yl)phenyl)methyl)phosphonic acid (443 mg, 1.11 mmol, 50%) as a white solid. LCMS (ESI): m/z=400 [M+H]⁺.

### Step31: Preparation of (E)-(((difluoro(4-(3-(4-nitrophenoxy)-3-oxoprop-1-en-1-yl)phenyl)methyl)phosphoryl)bis(oxy))bis(methylene)bis(2,2-dimethylpropanoate)

To a solution of (E)-(difluoro(4-(3-(4-nitrophenoxy)-3-oxoprop-1-en-1-yl)phenyl)methyl)phosphonic acid (443 mg, 1.11 mmol, 1.0 eq.) in a mixture of deionized H₂O (6 mL) and THF (3 mL) was added Amberlite IR120® resin (Na⁺ form) (2.5 g). The resulting mixture was stirred at room temperature for 1 h, then the excess of the resin was removed by filtration. AgNO₃ (755 mg, 4.44 mmol, 4.0 eq.) in deionized H₂O (2 mL) was then added to the resulting solution. After addition, the resulting mixture was stirred at room temperature for an additional 1 hr. During this period, the silver salt formed as a white precipitate, which was collected via filtration. The filter cake was washed with cool H₂O (2 mL×3), and the silver salt was further dried under reduced pressure to give a dry powder, which was used without further purification.

The isolated Ag salt was suspended in anhydrous toluene (10 mL), and iodomethyl pivalate (806 mg, 3.33 mmol, 3.0 eq.) was added dropwise. After addition, the resulting mixture was stirred at room temperature for an additional 12 h. The reaction progress was monitored by LCMS, and after completion, the unreacted silver salt was recovered by filtration, and the filtrate was concentrated under reduced pressure. The resulting residue was purified by flash column chromatography on silica gel to afford (E)-(((difluoro(4-(3-(4-nitrophenoxy)-3-oxoprop-1-en-1-yl)phenyl)methyl)phosphoryl)bis(oxy))bis(methylene)bis(2,2-dimethylpropanoate) (297 mg, 0.47 mmol, 43%) as a white solid. LCMS (ESI) m/z=628 [M+H]⁺.

### Synthesis of 4-nitrophenyl (E)-3-(4-((bis(2-(butyrylthio)ethoxy)phosphoryl)difluoromethyl)phenyl)acrylate

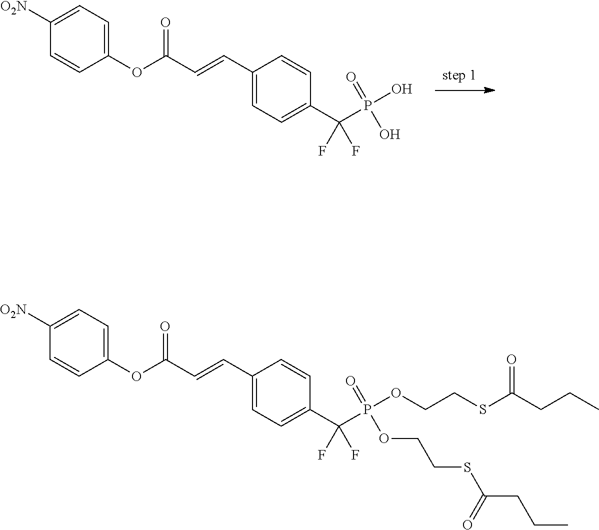 [SMILES: O=C(/C=C/c1ccc(C(F)(F)P(=O)(O)O)cc1)Oc1ccc([N+](=O)[O-])cc1 ; CCCC(=O)SCCOP(=O)(OCCSC(=O)CCC)C(F)(F)c1ccc(/C=C/C(=O)Oc2ccc([N+](=O)[O-])cc2)cc1]

### Step 1: Preparation of 4-nitrophenyl (E)-3-(4-((bis(2-(butyrylthio)ethoxy)phosphoryl)difluoromethyl)phenyl)acrylate

Oxalyl chloride (284 mg, 2.26 mmol, 3 eq.) was added dropwise to the solution of (E)-(difluoro(4-(3-(4-nitrophenoxy)-3-oxoprop-1-en-1-yl)phenyl)methyl)phosphonic acid (300 mg, 0.75 mmol, 1 eq.) (prepared according procedures adapted from JMC (2011) 54, 3549-3563) in dry DCM (5 mL) and DMF (0.1 mL) at 0° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (10 mL), then added to a mixture of S-(2-hydroxyethyl) butanethioate (334 mg, 2.26 mmol, 3 eq.) and TEA (379 mg, 3.75 mmol, 5 eq.) in anhydrous DCM (10 mL) at 0° C. The reaction was allowed to warm to 25° C. and stirred for an additional 2 h. After completion, the reaction mixture was concentrated under reduced pressure. The residue was purified by flash column chromatography on silica gel to afford 4-nitrophenyl (E)-3-(4-((bis(2-(butyrylthio)ethoxy)phosphoryl)difluoromethyl)phenyl)acrylate (208 mg, 0.32 mmol, 43%) as a colorless oil. LCMS (ESI): m/z=660 [M+H]⁺.

### Synthesis of 4-nitrophenyl 5-(((3-cyanophenoxy)(2-((3-methylbutanoyl)thio)ethoxy)phosphoryl)difluoromethyl)benzo[b]thiophene-2-carboxylate

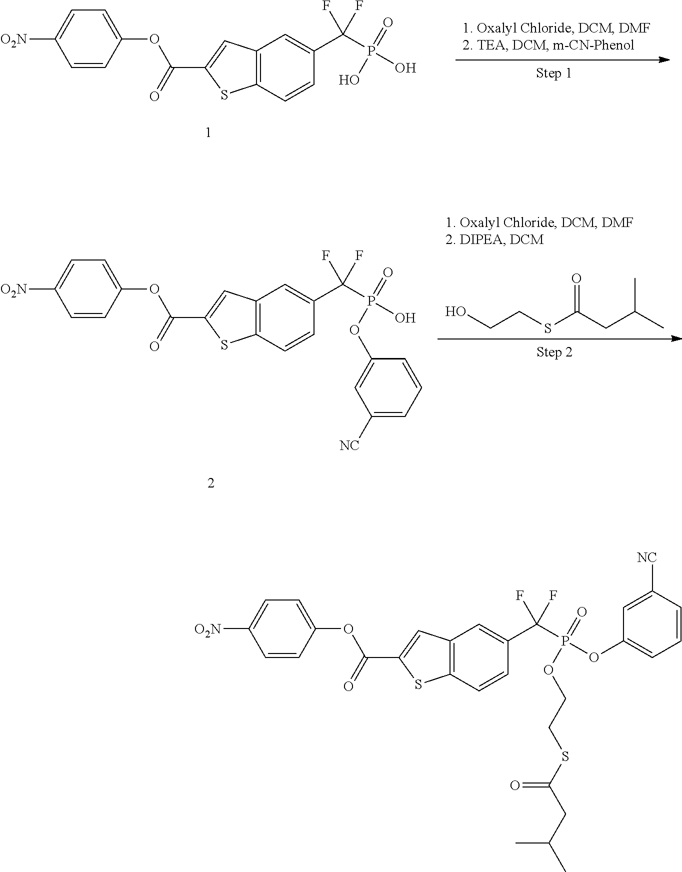

### Step 1: Preparation of 4-nitrophenyl 5-(((3-cyanophenoxy)(hydroxy)phosphoryl)difluoromethyl)benzo[b]thiophene-2-carboxylate

To a solution of (difluoro(2-((4-nitrophenoxy)carbonyl)benzo[b]thiophen-5-yl)methyl)phosphonic acid (600 mg, 1.39 mmol, 1 eq) in DCM (10 mL) at 0° C. were added 3 drops of DMF (cat) followed by dropwise addition of oxalyl chloride (1.77 mL, 20.8 mmol, 15 eq) The reaction was warmed up to room temperature and stirred for 2 h. The reaction was concentrated under reduced pressure and dried completely under high vacuum to give a beige solid which was diluted in DCM (10 mL) and cooled down to −78° C. A solution of 3-hydroxybenzonitrile (165 mg, 1.39 mmol, 1 eq) and triethylamine (387 μL, 2.78 mmol, 2 eq) in DCM (1 mL) was slowly added over 5 mi. The reaction mixture was stirred at −78° C. for 20 min. and then warmed up to room temperature and stirred for 18 h. More triethylamine (190 μL, 1 eq) was added as a solution in DCM (1.5 mL) and the reaction was stirred for 3 h. More triethylamine (190 μL, 1 eq) was added as a solution in DCM (1.5 mL) and the reaction was stirred for 18 h. Water (2-3 drops) was added and the reaction was concentrated under reduced pressure. The crude residue was purified by reverse phase chromatography on a 100 g C₁₈ cartridge eluting with a gradient of 5-100% acetonitrile in water to give 4-nitrophenyl 5-(((3-cyanophenoxy)(hydroxy)phosphoryl)difluoromethyl)benzo[b]thiophene-2-carboxylate (372 mg, 42.4%) as a yellow solid. LCMS (ESI) m/z=528.8 (M−H)⁻. ¹H NMR (400 MHz, CDCl₃) δ 11.66-11.40 (m, 1H), 8.39-8.32 (m, 2H), 8.31-8.22 (m, 2H), 7.96-7.82 (m, 2H), 7.53-7.46 (m, 3H), 7.45-7.39 (m, 1H), 7.32-7.28 (m, 2H), 3.09-2.96 (m, 6H), 1.27 (t, J=7.3 Hz, 9H).

### Step 2: Preparation of 4-nitrophenyl 5-(((3-cyanophenoxy)(2-((3-methylbutanoyl)thio)ethoxy)phosphoryl)difluoromethyl)benzo[b]thiophene-2-carboxylate

To a solution of 4-nitrophenyl 5-(((3-cyanophenoxy)(hydroxy)phosphoryl)difluoromethyl)benzo[b]thiophene-2-carboxylate (372 mg, 588 μmol, 1 eq) in DCM (6 mL) at 0° C. were added 1 drop of DMF (cat) followed by dropwise addition of oxalyl chloride (750 μL, 8.82 mmol, 15 eq). The reaction was warmed up to room temperature and stirred for 1.5 h. The reaction was concentrated under reduced pressure and dried completely under high vacuum for 30 min to give a yellow solid. The yellow solid was diluted in DCM (6 mL) and cooled down to 0° C. A solution of 1-[(2-hydroxyethyl)sulfanyl]-3-methylbutan-1-one (95.4 mg, 588 μmol, 1 eq) and N,N-diisopropylethylamine (0.3 mL, 1.76 mmol, 3 eq) in DCM (1 mL) was dried on sodium sulfate and slowly added on the yellow solution. The reaction mixture was stirred at 0° C. for 5 min. and warmed up to room temperature and stirred for 18 h. A solution of N,N-diisopropylethylamine (0.30 mL, 1.76 mmol, 3 eq) and 1-[(2-hydroxyethyl)sulfanyl]-3-methylbutan-1-one (300 mg, 1.76 mmol, 3 eq) in DCM (4 mL) was added dropwise at 0° C. and stirred for 1.5 h at room temperature. Water (2 mL) was added and the reaction was concentrated under reduced pressure. The crude residue was purified by reverse phase chromatography on a 50 g C₁₈ cartridge eluting with a gradient of 5-100% acetonitrile in water to give 4-nitrophenyl 5-(((3-cyanophenoxy)(2-((3-methylbutanoyl)thio)ethoxy)phosphoryl)difluoromethyl)benzo[b]thiophene-2-carboxylate (133 mg, 33.5%) as a yellow oil. ¹H NMR (400 MHz, CDCl₃) δ 8.40-8.29 (m, 3H), 8.28-8.23 (m, 1H), 8.11-8.00 (m, 1H), 7.86-7.72 (m, 1H), 7.55-7.40 (m, 6H), 4.44-4.19 (m, 2H), 3.25-3.02 (m, 2H), 2.54-2.33 (m, 2H), 2.22-2.06 (m, 1H), 0.95 (d, J=6.8 Hz, 6H).

### Synthesis of 4-nitrophenyl 5-(difluoro((((isopropoxycarbonyl)oxy)methoxy)(2-(pivaloylthio)ethoxy)phosphoryl)methyl)benzo[b]thiophene-2-carboxylate

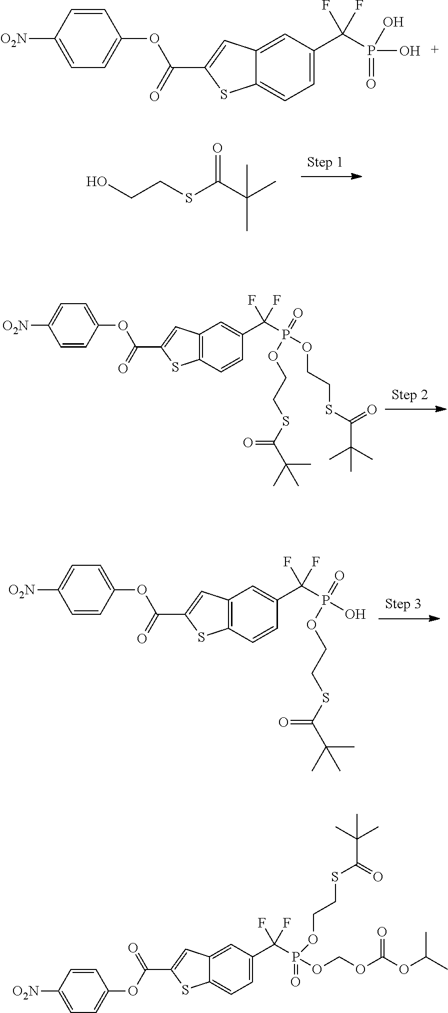

### Step 1: Preparation of 4-nitrophenyl 5-((bis(2-(pivaloylthio)ethoxy)phosphoryl)difluoromethyl)benzo[b]thiophene-2-carboxylate

To a solution of (difluoro(2-((4-nitrophenoxy)carbonyl)benzo[b]thiophen-5-yl)methyl)phosphonic acid (1 g, 2.32 mmol, 1 eq) in DCM (25 mL) at 0° C. were added 5 drops of DMF (cat.) followed by dropwise addition of oxalyl chloride (1.98 mL, 23.2 mmol, 10 eq). The reaction mixture was warmed up to room temperature and stirred for 2 h. The reaction mixture was not soluble at first (yellow solid floating on surface) but became a clear solution upon adding oxalyl chloride and warming up to room temperature. The reaction mixture was concentrated under reduced pressure and dried completely under high vacuum for 30 min. to give a yellow solid. The yellow solid was diluted in DCM (25 mL) and cooled down to 0° C. A solution of S-(2-hydroxyethyl) 2,2-dimethylpropanethioate (1.12 g, 6.96 mmol, 3 eq) in DCM (2 mL, dried on Na₂SO₄) was added dropwise and then triethylamine (969 μL, 6.96 mmol, 3 eq) in DCM (2 mL, dried on Na₂SO₄) was slowly added on the yellow solution over 1 min. The reaction mixture was stirred at 0° C. for 5 min., then warmed up to room temperature and stirred for 20 h. Water (1 mL) was added and the reaction mixture was concentrated under reduced pressure. The crude residue was purified by reverse phase chromatography on a 100 g C₁₈ cartridge eluting with 5-100% MeCN in water to give 4-nitrophenyl 5-((bis(2-(pivaloylthio)ethoxy)phosphoryl)difluoromethyl)benzo[b]thiophene-2-carboxylate (1.19 g, 71.6%) as a yellow semi-solid. ¹H NMR (400 MHz, DMSO-d₆) δ 8.67-8.63 (m, 1H), 8.42-8.34 (m, 4H), 7.75-7.64 (m, 3H), 4.23-4.09 (m, 4H), 3.17-3.07 (m, 4H), 1.18-1.13 (m, 18H).

### Step 2: Preparation of 4-nitrophenyl 5-(difluoro(hydroxy(2-(pivaloylthio)ethoxy)phosphoryl)methyl)benzo[b]thiophene-2-carboxylate

A solution of 4-nitrophenyl 5-((bis(2-(pivaloylthio)ethoxy)phosphoryl)difluoromethyl)benzo[b]thiophene-2-carboxylate (1.19 g, 1.65 mmol, 1 eq) in acetonitrile (3 mL) and water (3 mL) was heated to 75° C. for 48 h. The crude product was purified by reverse phase chromatography on a 100 g C₁₈ cartridge eluting with 5-100% MeCN in water. The pure fractions were concentrated under reduced pressure and freeze-dried to give 4-nitrophenyl 5-(difluoro(hydroxy(2-(pivaloylthio)ethoxy)phosphoryl)methyl)benzo[b]thiophene-2-carboxylate (512 mg, 54.1%) as a white solid. ¹H NMR (400 MHz, DMSO-d₆) δ 8.67-8.62 (m, 1H), 8.42-8.24 (m, 4H), 7.77-7.65 (m, 3H), 3.98-3.93 (m, 2H), 3.03 (t, J=6.5 Hz, 2H), 1.17 (s, 9H).

### Step 3: Preparation of 4-nitrophenyl 5-(difluoro((((isopropoxycarbonyl)oxy)methoxy)(2-(pivaloylthio)ethoxy)phosphoryl)methyl)benzo[b]thiophene-2-carboxylate

A mixture of 4-nitrophenyl 5-(difluoro(hydroxy(2-(pivaloylthio)ethoxy)phosphoryl)methyl)benzo[b]thiophene-2-carboxylate (350 mg, 0.6102 mmol, 1 eq) and Amberlite® IR120 Na⁺ form (1.75 g) in deionized water (10 mL) and THF (10 mL) was stirred at room temperature for 4 h. The mixture was filtered. Silver nitrate (414 mg, 2.44 mmol, 4 eq) was added into the filtrate. After addition, the reaction mixture was stirred at room temperature for 1 h. The mixture was filtered and the filter cake was transferred in a separate flask with acetonitrile (20 mL), concentrated under reduced pressure and dried under high vacuum for 2 h. The solid was suspended in acetonitrile (10 mL) and chloromethyl propan-2-yl carbonate (465 mg, 3.05 mmol, 5 eq) was added. After addition, the reaction mixture was stirred at room temperature overnight. Additional chloromethyl propan-2-yl carbonate (465 mg, 3.05 mmol, 5 eq) was added and the reaction was stirred at 45° C. for 5 h. Celite was added and the reaction was concentrated under reduced pressure. The crude residue was purified by flash-chromatography on silica gel eluting with 0-50% EtOAc in heptane to give 4-nitrophenyl 5-(difluoro((((isopropoxycarbonyl)oxy)methoxy)(2-(pivaloylthio)ethoxy)phosphoryl)methyl)benzo[b]thiophene-2-carboxylate (92.0 mg, 21.9%) as a yellowish oil. ¹H NMR (400 MHz, CDCl₃) δ 8.04-8.34 (m, 3H), 8.25 (s, 1H), 8.05 (d, J=8.5 Hz, 1H), 7.78 (d, J=8.5 Hz, 1H), 7.51 (d, J=9.3 Hz, 2H), 5.79-5.65 (m, 2H), 8.00-4.93 (m, 1H), 4.32-4.20 (m, 2H), 3.17-3.11 (m, 2H), 1.36 (d, J=6.2 Hz, 6H), 1.25 (s, 9H).

### Synthesis of perfluorophenyl 5-(difluoro((2-((3-methylbutanoyl)thio)ethoxy)(((((tetrahydro-2H-pyran-4-yl)oxy)carbonyl)oxy)methoxy)phosphoryl)methyl)benzo[b]thiophene-2-carboxylate

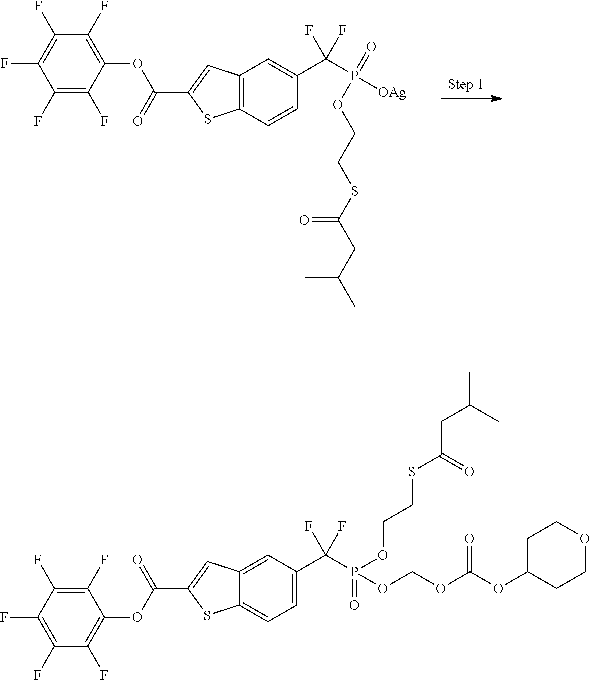

### Step 1: Preparation of Perfluorophenyl 5-(difluoro((2-((3-methylbutanoyl)thio)ethoxy)(((((tetrahydro-2H-pyran-4-yl)oxy)carbonyl)oxy)methoxy)phosphoryl)methyl)benzo[b]thiophene-2-carboxylate

To a solution of (((difluoro(2-((perfluorophenoxy)carbonyl)benzo[b]thiophen-5-yl)methyl)(2-((3-methylbutanoyl)thio)ethoxy)phosphoryl)oxy)silver [prepared using the protocols shown in step 1 and 2 from the synthesis of 4-nitrophenyl 5-(difluoro((((isopropoxycarbonyl)oxy)methoxy)(2-(pivaloylthio)ethoxy)phosphoryl)methyl)benzo[b]thiophene-2-carboxylate and utilizing the appropriate starting materials and modifications](150 mg, 0.21 mmol, 1.0 eq.) in MeCN (3 mL) was added chloromethyl (tetrahydro-2H-pyran-4-yl) carbonate (62 mg, 0.32 mmol, 1.5 eq.) was added. The mixture was stirred at room temperature for 24 h. After filtration, the solvent was removed in vacuo. The crude residue was directly purified by flash column chromatography on silica gel to give perfluorophenyl 5-(difluoro((2-((3-methylbutanoyl)thio)ethoxy)(((((tetrahydro-2H-pyran-4-yl)oxy)carbonyl)oxy)methoxy)phosphoryl)methyl)benzo[b]thiophene-2-carboxylate (15 mg, 19 μmol, 9%) as a white solid. LCMS (ESI): m/z=777 [M+H]⁺.

The following intermediates in Table 51 were prepared using the method described above for the synthesis of perfluorophenyl 5-(difluoro((2-((3-methylbutanoyl)thio)ethoxy)(((((tetrahydro-2H-pyran-4-yl)oxy)carbonyl)oxy)methoxy)phosphoryl)methyl)benzo[b]thiophene-2-carboxylate and utilizing the appropriate starting materials and modifications.

TABLE 51

| Name | Structure | LCMS |
|---|---|---|
| perfluorophenyl 5-(difluoro((2-((3- methylbutanoyl)thio)ethoxy)((((((S)- THF-3- yl)oxy)carbonyl)oxy)methoxy) phosphoryl)methyl) benzo[b]thiophene-2- carboxylate | 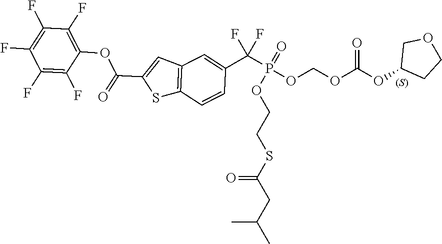 | 785 [M + Na]⁺ |
| perfluorophenyl 5-(difluoro((2-((3- methylbutanoyl)thio)ethoxy)((((((R)- THF-3- yl)oxy)carbonyl)oxy)methoxy) phosphoryl)methyl)benzo[b]thiophene-2- carboxylate | 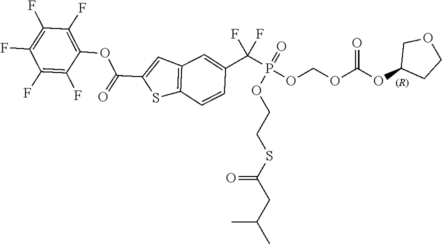 | 763 [M + H]⁺ |

### Synthesis of 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylic acid

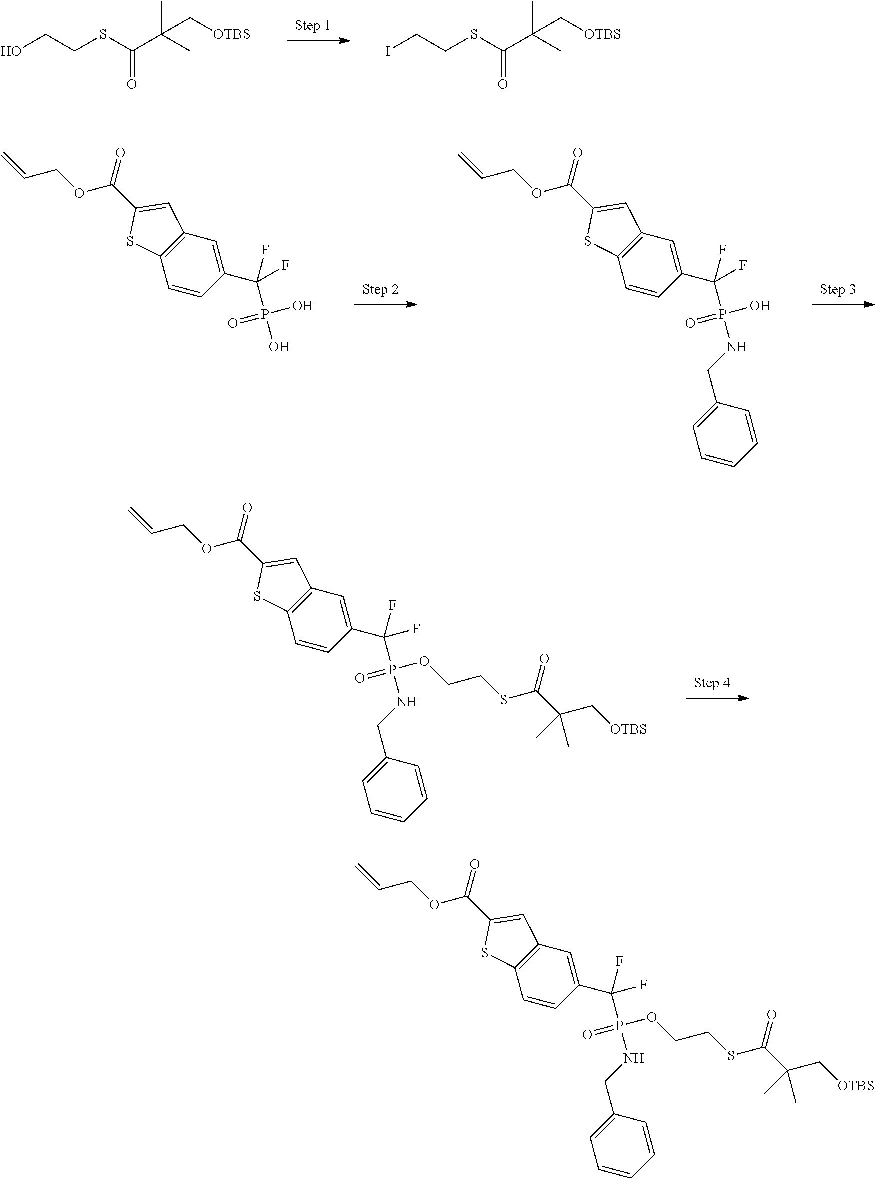 [SMILES: CC(C)(CO[Si](C)(C)C(C)(C)C)C(=O)SCCO ; CC(C)(CO[Si](C)(C)C(C)(C)C)C(=O)SCCI ; C=CCOC(=O)c1cc2cc(C(F)(F)P(=O)(O)O)ccc2s1 ; C=CCOC(=O)c1cc2cc(C(F)(F)P(=O)(O)NCc3ccccc3)ccc2s1 ; C=CCOC(=O)c1cc2cc(C(F)(F)P(=O)(NCc3ccccc3)OCCSC(=O)C(C)(C)CO[Si](C)(C)C(C)(C)C)ccc2s1 ; C=CCOC(=O)c1cc2cc(C(F)(F)P(=O)(NCc3ccccc3)OCCSC(=O)C(C)(C)CO[Si](C)(C)C(C)(C)C)ccc2s1]

### Step 1: Preparation of S-(2-iodoethyl) 3-((tert-butyldimethylsilyl)oxy)-2,2-dimethylpropanethioate

To a solution of S-(2-hydroxyethyl) 3-((tert-butyldimethylsilyl)oxy)-2,2-dimethylpropanethioate (700 mg, 2.39 mmol, 1 eq.) in DCM (20 mL) was added PPh₃ (626 mg, 2.39 mmol, 1.0 eq.) portionwise at 0° C. and the resulting mixture was stirred for 30 min. Then NIS (537 mg, 2.39 mmol, 1.0 eq) was added dropwise at 0° C. and the reaction mixture was stirred at 0° C. for 1 hr. After completion, the reaction mixture was concentrated under reduced pressure. The residue was purified by flash column chromatography on silica gel to give S-(2-iodoethyl) 3-((tert-butyldimethylsilyl)oxy)-2,2-dimethylpropanethioate (700 mg, 1.73 mmol, 73%) as a clear oil. ¹H NMR (400 MHz, CDCl₃) δ 3.57 (s, 2H), 3.31-3.24 (m, 2H), 3.23-3.15 (m, 2H), 1.17 (s, 6H), 0.84 (s, 9H), 0.00 (s, 6H).

### Step 2: Preparation of P-((2-((allyloxy)carbonyl)benzo[b]thiophen-5-yl)difluoromethyl)-N-benzylphosphonamidic acid

Oxalyl chloride (1.01 g, 8.00 mmol, 5 eq.) was added dropwise to the solution of ((2-((allyloxy)carbonyl)benzo[b]thiophen-5-yl)difluoromethyl)phosphonic acid (150 mg, 0.4307 mmol, 1 eq.) in dry DCM (10 mL) and DMF (1 drop) at 0° C. The reaction mixture was stirred at 40° C. for an additional 2 h. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (10 mL), then added to a mixture of phenylmethanamine (46.0 mg, 430 μmol, 1 eq.) and TEA (217 mg, 2.15 mmol, 5 eq.) in anhydrous DCM (10 mL) at 0° C. The reaction was allowed to warm to 25° C. and stirred for an additional 2 h. After completion, the reaction mixture was concentrated under reduced pressure. The residue was purified by Biotage® C18 column chromatography to give P-((2-((allyloxy)carbonyl)benzo[b]thiophen-5-yl)difluoromethyl)-N-benzylphosphonamidic acid (100 mg, 0.23 mmol, 53%) as a colorless oil. LCMS (ESI): m/z=438 [M+H]⁺.

### Step 3: Preparation of Allyl 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylate

Amberlite IR120® (18.2 mg, 457 μmol, 2 eq) in water (2 mL) was added dropwise to a stirred suspension of P-((2-((allyloxy)carbonyl)benzo[b]thiophen-5-yl)difluoromethyl)-N-benzylphosphonamidic acid (100 mg, 0.23 mmol, 1 eq.) in H₂O (6 mL). When the mixture became clear (pH˜9), silver(I) nitrate (85.2 mg, 502 μmol, 2.2 eq.) was added. After stirring at 0° C. for 2 h, the gray precipitate was collected by filtration and dried under vacuum. The powder was suspended in dry Toluene (1 mL) and S-(2-iodoethyl) 3-((tert-butyldimethylsilyl)oxy)-2,2-dimethylpropanethioate (1.68 g, 6.96 mmol, 30 eq.) was added. The mixture was stirred at 80° C. for 24 h. After filtration, the solvent was removed in vacuo. The crude residue was directly purified by flash column chromatography on silica gel to give allyl 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylate (40.0 mg, 56 μmol, 25%) as a white solid. LCMS (ESI): m/z=734.2 [M+Na]⁺.

### Step 4: Preparation of 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylic acid

To a solution of allyl 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylate (35 mg, 49 μmol, 1 eq.) in DCM (1 mL) were added Pd(PPh₃)₄(2.83 mg, 2.45 μmol, 0.05 eq.) and pyrrolidine (3 mg, 49.1 μmol, 1 eq.). The mixture was purged and degassed with N₂ (three times), then stirred at 25° C. for 2 h. After completion, the mixture was concentrated under reduced pressure. The residue was purified by Biotage® C18 column chromatography to give 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylic acid (20.0 mg, 30 μmol, 61%) as a colorless oil. LCMS (ESI): m/z=672.1 [M+H]⁺.

### Synthesis of 5-(((((S)-1-(benzyloxy)-1-oxopropan-2-yl)amino)(phenoxy)phosphoryl)difluoromethyl)benzo[b]thiophene-2-carboxylic acid

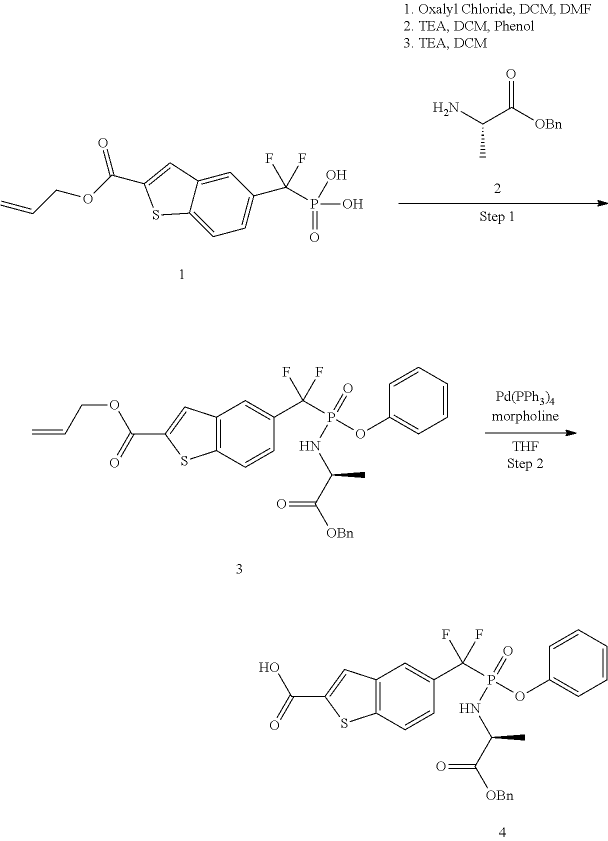 [SMILES: C=CCOC(=O)c1cc2cc(C(F)(F)P(=O)(O)O)ccc2s1 ; C[C@H](N)C(=O)OCc1ccccc1 ; C=CCOC(=O)c1cc2cc(C(F)(F)P(=O)(N[C@@H](C)C(=O)OCc3ccccc3)Oc3ccccc3)ccc2s1 ; C[C@H](NP(=O)(Oc1ccccc1)C(F)(F)c1ccc2sc(C(=O)O)cc2c1)C(=O)OCc1ccccc1]

### Step 1: Preparation of Allyl 5-(((((S)-1-(benzyloxy)-1-oxopropan-2-yl)amino)(phenoxy)phosphoryl)difluoromethyl)benzo[b]thiophene-2-carboxylate

To a solution of ((2-((allyloxy)carbonyl)benzo[b]thiophen-5-yl)difluoromethyl)phosphonic acid (300 mg, 861 μmol, 1 eq) in DCM (5 mL) at 0° C. were added 2 drops of DMF (cat.) followed by dropwise addition of oxalyl chloride (220 μL, 2.58 mmol, 3 eq). The reaction was warmed up to room temperature and stirred for 16 h. The reaction mixture was concentrated under reduced pressure and dried completely under high vacuum for 30 min. to give a yellow solid. The yellow solid was diluted in DCM (5 mL) and cooled to 0° C. A solution of phenol (89.1 mg, 947 μmol, 1.1 eq) and triethylamine (599 μL, 4.30 mmol, 5 eq) in DCM (1 mL, dried on Na₂SO₄) was slowly added to the yellow solution. The reaction mixture was stirred at 0° C. for 2 min. and then warmed up to room temperature and stirred for 2 h. A solution of L-alanine benzyl ester p-toluenesulfonate salt (453 mg, 1.29 mmol, 1.5 eq) in DCM (1 mL, dried on Na₂SO₄) was slowly added to the yellow solution. The reaction mixture was stirred at room temperature for 18 h. The solvent was removed in vacuo. The crude product was purified by reverse phase chromatography on a 50 g C₁₈ cartridge eluting with a gradient of 5-100% acetonitrile in water. The pure fractions were combined and concentrated under reduced pressure to give allyl 5-(((((S)-1-(benzyloxy)-1-oxopropan-2-yl)amino)(phenoxy)phosphoryl)difluoromethyl)benzo[b]thiophene-2-carboxylate (196 mg, 38.8%) as a yellow oil. LCMS (ESI) m/z=586.2.

The following intermediates in Table 52 were prepared using the method described above in step 1 for the preparation of allyl 5-(((((S)-1-(benzyloxy)-1-oxopropan-2-yl)amino)(phenoxy)phosphoryl)difluoromethyl)benzo[b]thiophene-2-carboxylate and utilizing the appropriate starting materials and modifications.

TABLE 52

| Name | Structure | LCMS | NMR |
|---|---|---|---|
| butyl (2S)-1-(((2- ((allyloxy)carbonyl) benzo[b]thiophen-5- yl)difluoromethyl) (phenoxy)phosphoryl)- 4,4- difluoropyrrolidine-2- carboxylate | 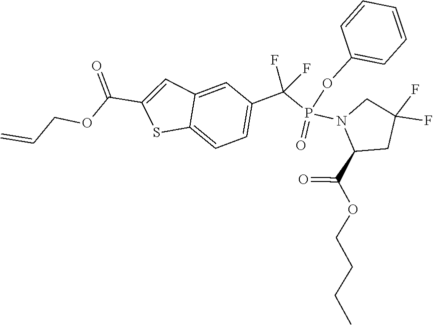 [SMILES: C=CCOC(=O)c1cc2cc(C(F)(F)P(=O)(Oc3ccccc3)N3CC(F)(F)C[C@H]3C(=O)OCCCC)ccc2s1] | 614.2 [M + H]⁺ | ¹H NMR (400 MHz, CDCl₃) δ 8.19 (s, 1H), 8.12 (s, 1H), 7.99 − 7.91 (m, 1H), 7.79 − 7.73 (m, 1H), 7.35 − 7.02 (m, 5H), 6.12 − 5.98 (m, 1H), 5.50 − 5.41 (m, 1H), 5.38 − 5.28 (m, 1H), 4.89 − 4.84 (m, 2H), 4.81 − 4.73 (m, 0.8H), 4.47 − 4.38 (m, 0.2H), 4.29 − 3.96 (m, 2H), 3.88 − 3.64 (m, 2H), 2.76 − 2.43 (m, 2H), 1.61 − 1.50 (m, 2H), 1.39 − 1.23 (m, 2H), 0.90 (t, J = 7.3 Hz, 3H) |
| butyl (((2- ((allyloxy)carbonyl) benzo[b]thiophen-5- yl)difluoromethyl) (phenoxy)phosphoryl)- L-prolinate | 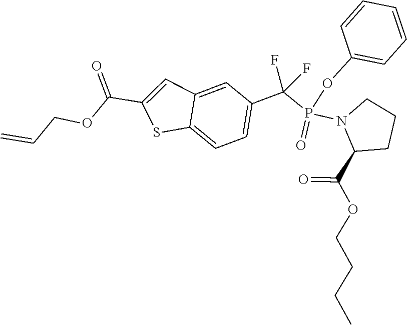 | 542.3 [M + H]⁺ | |

### Step 2: Preparation of 5-(((((S)-1-(benzyloxy)-1-oxopropan-2-yl)amino)(phenoxy)phosphoryl)difluoromethyl)benzo[b]thiophene-2-carboxylic acid

To a stirred solution of allyl 5-(((((S)-1-(benzyloxy)-1-oxopropan-2-yl)amino)(phenoxy)phosphoryl)difluoromethyl)benzo[b]thiophene-2-carboxylate (196 mg, 334 μmol, 1 eq) in THF (5 mL) were added morpholine (143 μL, 1.67 mmol, 5 eq) and Pd(PPh₃)₄(38.5 mg, 33.4 μmol, 0.10 eq) under nitrogen. The reaction mixture was stirred 1 h at room temperature. The reaction mixture was directly purified by reverse phase chromatography on a 50 g C₁₈ cartridge eluting with a gradient of 5-100% acetonitrile in water (with 0.1% formic acid). The pure fractions were concentrated under reduced pressure to give 5-(((((S)-1-(benzyloxy)-1-oxopropan-2-yl)amino) (phenoxy)phosphoryl)difluoromethyl)benzo[b]thiophene-2-carboxylic acid (164 mg, 90.1%) as a light brown solid. ¹H NMR (400 MHz, CDCl₃) δ 8.22-8.09 (m, 1H), 7.88-7.76 (m, 2H), 7.66-7.59 (m, 1H), 7.43-7.29 (m, 7.6H), 7.26-7.15 (m, 2.4H), 5.24 (s, 1H), 5.13 (s, 1H), 4.83-4.67 (m, 0.5H), 4.62-4.44 (m, 0.5H), 4.39-4.14 (m, 1H), 1.50-1.35 (m, 3H).

The following intermediates in Table 53 were prepared using the method described above in step 2 for the preparation of 5-(((((S)-1-(benzyloxy)-1-oxopropan-2-yl)amino)(phenoxy)phosphoryl)difluoromethyl)benzo[b]thiophene-2-carboxylic acid and utilizing the appropriate starting materials and modifications.

TABLE 53

| Name | Structure | LCMS |
|---|---|---|
| 5-((((S)-2-(butoxycarbonyl)-4,4- difluoropyrrolidin-1- yl)(phenoxy)phosphoryl)difluoromethyl)benzo [b]thiophene-2-carboxylic acid | 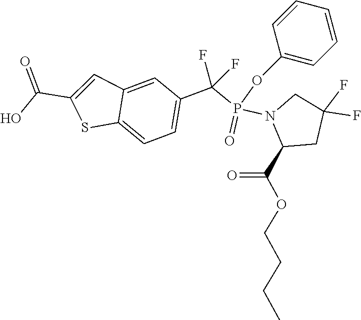 [SMILES: CCCCOC(=O)[C@@H]1CC(F)(F)CN1P(=O)(Oc1ccccc1)C(F)(F)c1ccc2sc(C(=O)O)cc2c1] | 574.2 [M + H]⁺ |
| 5-((((S)-2-(butoxycarbonyl)pyrrolidin-1- yl)(phenoxy)phosphoryl)difluoromethyl)benzo [b]thiophene-2-carboxylic acid | 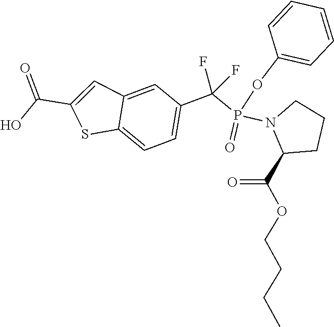 [SMILES: CCCCOC(=O)[C@@H]1CCCN1P(=O)(Oc1ccccc1)C(F)(F)c1ccc2sc(C(=O)O)cc2c1] | 537.8 [M + H]⁺ |

### Synthesis of 5-(((((S)-1-butoxy-1-oxopropan-2-yl)amino)(naphthalen-1-yloxy)phosphoryl)difluoromethyl)benzo[b]thiophene-2-carboxylic acid

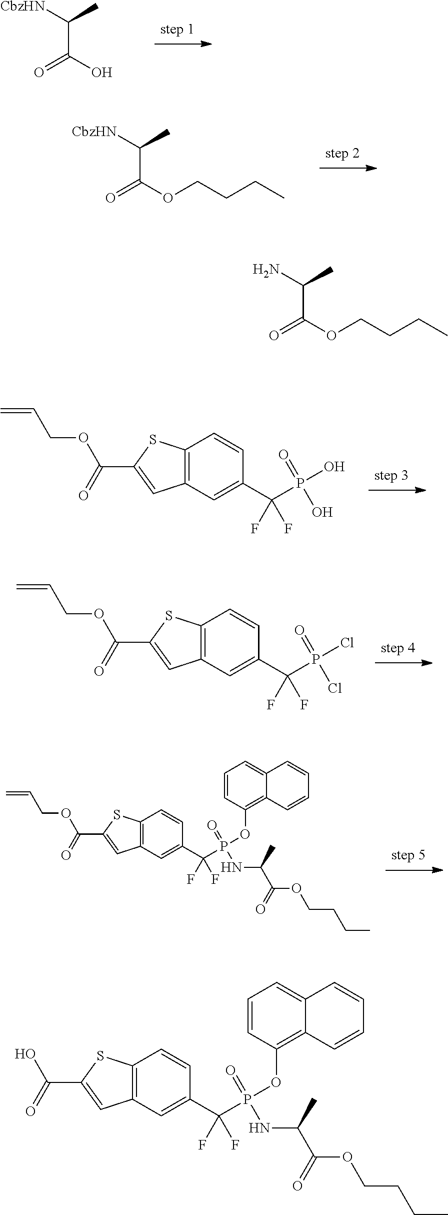

### Step 1: Preparation of Butyl ((benzyloxy)carbonyl)-L-alaninate

To a solution of ((benzyloxy)carbonyl)-L-alanine (5 g, 22 mmol, 1 eq) in DMF (50 mL) was added 1-chlorobutane (2.1 g, 22 mmol, 1 eq) and dipotassium carbonate (6.2 g, 45 mmol, 2 eq), the mixture was stirred at 60° C. for 1 h to give yellow solution. The mixture was diluted with water (200 mL) and extracted with EtOAc (200 mL×2), the combined organic layers were washed with saturated brine (200 mL×2), dried over Na₂SO₄, filtered and concentrated under reduced pressure to give a residue. The residue was purified by column chromatography to give butyl ((benzyloxy)carbonyl)-L-alaninate (3.6 g, 13 mmol, 57.8% yield) as a yellow oil. LCMS (ESI) m/z=280.0

### Step 2: Preparation of Butyl L-alaninate

To a solution of butyl ((benzyloxy)carbonyl)-L-alaninate (3 g, 11 mmol, 1 eq) in THF (30 mL) was added Pd/C (3 g, 10%) under N₂, the mixture was stirred at 25° C. for 2 h under H₂ (15 Psi). The reaction mixture was filtered and the filter was concentrated to give butyl L-alaninate (1.7 g, crude) as a yellow oil. LCMS (ESI) m/z=146.1 [M+H]⁺.

### Step 3: Preparation of Allyl 5-((dichlorophosphoryl)difluoromethyl)benzo[b]thiophene-2-carboxylate

To a solution of ((2-((allyloxy)carbonyl)benzo[b]thiophen-5-yl)difluoromethyl)phosphonic acid (0.2 g, 0.57 mmol, 1 eq) in DCM (5 mL) was added dimethylformamide (0.42 mg, 5.7 μmol, 0.01 eq), the mixture was stirred at 0° C. for 5 min, then a solution of oxalyl chloride (0.22 g, 1.7 mmol, 3 eq) in DCM (5 mL) was added to the mixture, the mixture was stirred at 40° C. for 15 min to give yellow solution. The reaction mixture was concentrated under reduced pressure to give allyl 5-((dichlorophosphoryl)difluoromethyl)benzo[b]thiophene-2-carboxylate (0.2 g, crude) as a yellow oil. LCMS (ESI) m/z=377.0 [M+H]⁺.

### Step 4: Preparation of Allyl 5-(((((S)-1-butoxy-1-oxopropan-2-yl)amino)(naphthalen-1-yloxy)phosphoryl)difluoromethyl)benzo[b]thiophene-2-carboxylate

To a solution of allyl 5-((dichlorophosphoryl)difluoromethyl)benzo[b]thiophene-2-carboxylate (0.2 g, 0.52 mmol, 1 eq) in DCM (10 mL) was added naphthalen-1-ol (60 mg, 0.42 mmol, 0.8 eq), the mixture was stirred at 0° C. for 5 min, then a solution of N,N-diisopropylethylamine (0.2 g, 1.6 mmol, 3.0 eq) in DCM (10 mL) was added to the mixture, then a solution of butyl L-alaninate (75 mg, 0.52 mmol, 1 eq) in DCM (10 mL) was added to the mixture, the mixture was stirred at 0° C. for 30 min to give yellow solution. The reaction mixture was concentrated under reduced pressure to give a residue. The residue was purified by column chromatography to give allyl 5-(((((S)-1-butoxy-1-oxopropan-2-yl)amino)(naphthalen-1-yloxy)phosphoryl)difluoromethyl)benzo[b]thiophene-2-carboxylate (0.15 g, 0.24 mmol, 46.4% yield) as a yellow oil. LCMS (ESI) m/z=602.1 [M+H]⁺.

### Step 5: Preparation of 5-(((((S)-1-butoxy-1-oxopropan-2-yl)amino)(naphthalen-1-yloxy)phosphoryl)difluoromethyl)benzo[b]thiophene-2-carboxylic acid

To a solution of allyl 5-(((((S)-1-butoxy-1-oxopropan-2-yl)amino)(naphthalen-1-yloxy)phosphoryl)difluoromethyl)benzo[b]thiophene-2-carboxylate (0.14 g, 0.23 mmol, 1 eq) in DCM (1.0 mL) was added Pd(PPh₃)₄(27 mg, 23 μmol, 0.1 eq), the mixture was stirred at 0° C. for 5 min, then pyrrolidine (17 mg, 0.23 mmol, 1 eq) was added to the mixture, the mixture was stirred at 25° C. for 5 min to give yellow solution. The reaction mixture was concentrated under reduced pressure to give a residue. The residue was purified by reversed phase (TFA) to give 5-(((((S)-1-butoxy-1-oxopropan-2-yl)amino)(naphthalen-1-yloxy)phosphoryl)difluoromethyl)benzo[b]thiophene-2-carboxylic acid (97 mg, 0.17 mmol, 74.6% yield) as a yellow oil. LCMS (ESI) m/z=562.1 [M+H]⁺.

### Synthesis of 7-(difluoro((((S)-1-oxo-1-propoxypropan-2-yl)amino)(phenoxy)phosphoryl)methyl)-2-naphthoic acid

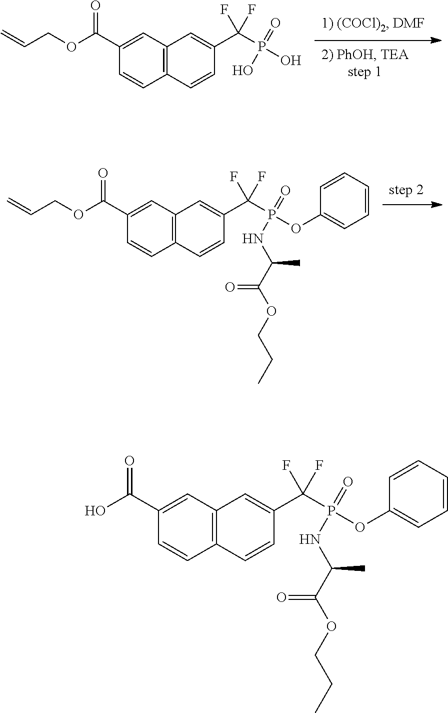 [SMILES: C=CCOC(=O)c1ccc2ccc(C(F)(F)P(=O)(O)O)cc2c1 ; C=CCOC(=O)c1ccc2ccc(C(F)(F)P(=O)(N[C@@H](C)C(=O)OCCC)Oc3ccccc3)cc2c1 ; CCCOC(=O)[C@H](C)NP(=O)(Oc1ccccc1)C(F)(F)c1ccc2ccc(C(=O)O)cc2c1]

### Step 1: Preparation of Allyl 7-(difluoro((((S)-1-oxo-1-propoxypropan-2-yl)amino)(phenoxy)phosphoryl)methyl)-2-naphthoate

Oxalyl chloride (10 mL) was added dropwise to the solution of ((7-((allyloxy)carbonyl)naphthalen-2-yl)difluoromethyl)phosphonic acid (1.5 g, 4.38 mmol, 1 eq.) in dry DCM (10 mL) and DMF (1 drop) at 0° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (5 mL), then added to a mixture of phenol (412 mg, 4.38 mmol, 1 eq.) and triethylamine (2.21 g, 21.9 mmol, 5 eq.) in anhydrous DCM (10 mL) at 0° C. The reaction was allowed to warm to 25° C. and stirred for an additional 1 hr. Then propyl L-alaninate (1.14 g, 8.76 mmol, 2 eq.) was added at 0° C. and the resulting mixture was stirred at 20° C. for 1 h under N₂. After completion, the reaction mixture was concentrated under reduced pressure. The residue was purified by Biotage® C18 column chromatography to give allyl 7-(difluoro((((S)-1-oxo-1-propoxypropan-2-yl)amino)(phenoxy)phosphoryl)methyl)-2-naphthoate (510 mg, 0.96 mmol, 22%) as a white solid. LCMS (ESI): m/z=532.2 [M+H]⁺.

The following intermediates in Table 54 were prepared using the method described above in step 1 for the preparation of 5-(((((S)-1-(benzyloxy)-1-oxopropan-2-yl)amino)(phenoxy)phosphoryl)difluoromethyl)benzo[b]thiophene-2-carboxylic acid and utilizing the appropriate starting materials and modifications.

TABLE 54

| Name | Structure | LCMS |
|---|---|---|
| allyl 7-(((((S)-1-butoxy-1-oxopropan-2- yl)amino)(naphthalen-1- yloxy)phosphoryl)difluoromethyl)-2- naphthoate | 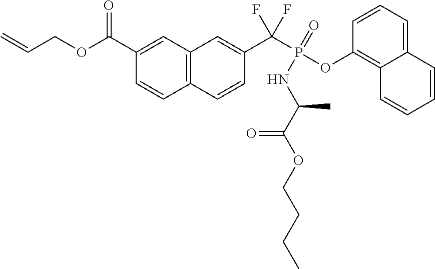 [SMILES: C=CCOC(=O)c1ccc2ccc(C(F)(F)P(=O)(N[C@@H](C)C(=O)OCCCC)Oc3cccc4ccccc34)cc2c1] | 596 [M + H]⁺ |

### Step 2: Preparation of 7-(difluoro((((S)-1-oxo-1-propoxypropan-2-yl)amino)(phenoxy)phosphoryl)methyl)-2-naphthoic acid

To a solution of allyl 7-(difluoro((((S)-1-oxo-1-propoxypropan-2-yl)amino)(phenoxy)phosphoryl)methyl)-2-naphthoate (2, 510 mg, 0.96 mmol, 1 eq) in DCM (2 mL) were added Pd(PPh₃)₄(22.3 mg, 0.01932 mmol, 0.05 eq.) and pyrrolidine (27.4 mg, 0.3864 mmol, 1.0 eq.). The mixture was purged and degassed with N₂ (three times), then stirred at 25° C. for 1 hr. After completion, the mixture was concentrated under reduced pressure. The residue was purified by Biotage® C18 column chromatography to give 7-(difluoro((((S)-1-oxo-1-propoxypropan-2-yl)amino)(phenoxy)phosphoryl)methyl)-2-naphthoic acid (1150 mg, 0.3142 mmol, 82%) as a white solid. LCMS (ESI): m/z=492 [M+H]⁺. ¹H NMR (400 MHz, DMSO) δ 8.75 (d, J=9.7 Hz, 1H), 8.44-8.38 (m, 1H), 8.21-8.14 (m, 1H), 8.13-8.06 (m, 2H), 7.83 (d, J=8.7 Hz, 1H), 7.40-7.31 (m, 2H), 7.26-7.10 (m, 3H), 6.88-6.78 (m, 1H), 4.00-3.90 (m, 1H), 3.90-3.76 (m, 2H), 1.55-1.38 (m, 2H), 1.25-1.17 (m, 3H), 0.86-0.74 (m, 3H).

The following intermediates in Table 55 were prepared using the method described above in step 2 for the preparation of 5-(((((S)-1-(benzyloxy)-1-oxopropan-2-yl)amino)(phenoxy)phosphoryl)difluoromethyl)benzo[b]thiophene-2-carboxylic acid and utilizing the appropriate starting materials and modifications.

TABLE 55

| Name | Structure | LCMS |
|---|---|---|
| 7-(((((S)-1-butoxy-1-oxopropan-2- yl)amino)(naphthalen-1- yloxy)phosphoryl)difluoromethyl)-2-naphthoic acid | 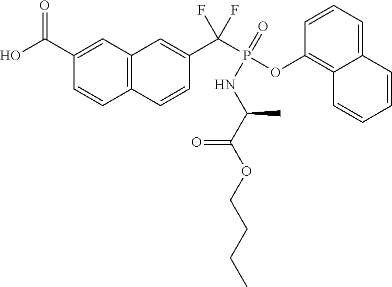 [SMILES: CCCCOC(=O)[C@H](C)NP(=O)(Oc1cccc2ccccc12)C(F)(F)c1ccc2ccc(C(=O)O)cc2c1] | 556 [M + H]⁺ |

### Synthesis of 7-((diethoxyphosphoryl)methyl)-2-naphthoic acid

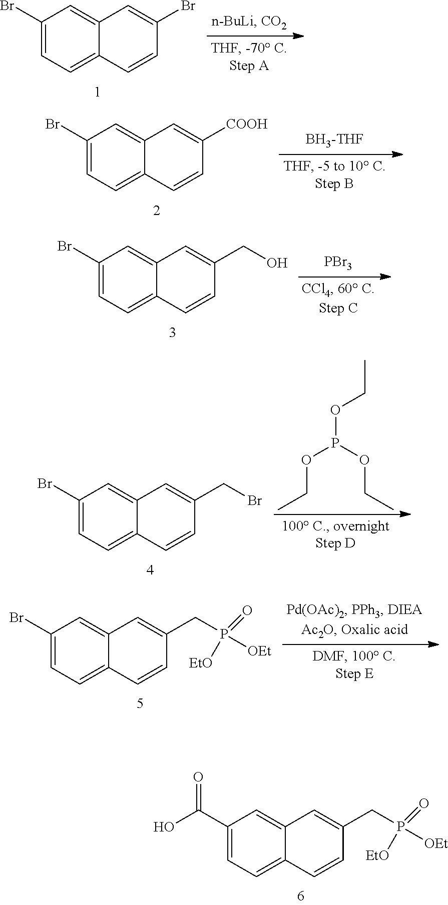

### Step A: 7-bromo-2-naphthoic acid

To a solution of 2,7-dibromonaphthalene (1, 400 g, 1.4 mol, 1.0 eq.) in THF (6000 mL) was added n-BuLi (560 mL, 1.4 mol, 2.5 M in hexane, 1.0 eq.) by dropping funnel over a period of 2.5 h at −70° C. under N₂. Then the reaction mixture was stirred at −70° C. for an additional 1 hr. The reaction was monitored by pipetting out a small amount of crude sample and quenching it with acetone to ensure 1 was consumed completely (observed by TLC). After completion, CO₂ (g) was bubbled into the reaction mixture at −70° C. to −60° C. for 30 minutes (3 L/min). The reaction progress was monitored by TLC (DCM:EA=1:1, R_{f}=0.2). After completion, the reaction mixture was quenched by adding H₂O (5000 mL), the suspension was filtered through a pad of celite and the filter cake was washed with H₂O (1000 mL). The combined filtrates were extracted with PE (3000 mL). The aqueous phase was acidified with 6 N HCl until the pH was adjusted to pH=3. The mixture was filtered and the filter cake was washed with H₂O (600 mL), then dried in vacuum to afford 7-bromo-2-naphthoic acid (2, 240 g, 0.96 mol, 68%) as a white solid. LCMS (ESI): m/z=251 [M+H]⁺.

### Step B: (7-bromonaphthalen-2-yl)methanol

To a solution of 7-bromo-2-naphthoic acid (2, 480 g, 1.92 mol, 1.0 eq.) in THF (1000 mL) was added BH₃/THF (4800 mL, 4.8 mol, 1 M in THF, 2.5 eq.) by dropping funnel over a period of 3 h at −20° C. to −5° C. under N₂. Then the reaction mixture was stirred at −5° C. to 10° C. overnight. The reaction progress was monitored by TLC (DCM:EA=5:1, R_{f}=0.3). After completion, the reaction mixture was quenched by adding HCl aqueous solution (1N, 400 mL) at 0° C. The mixture was diluted with H₂O (2000 mL) and extracted with EtOAc (2000 mL×2). The combined organic layers were washed with water (3000 mL), brine (2000 mL) and dried over anhydrous Na₂SO₄. The organic layers were concentrated under reduced pressure. The residue was purified by re-crystallization from DCE (2000 mL). The mixture was filtered and the filter cake was washed with PE (200 mL), then dried in vacuum to afford (7-bromonaphthalen-2-yl)methanol (3, 405 g, 1.70 mol, 89%) as a white solid.

### Step C: 2-bromo-7-(bromomethyl)naphthalene

To a solution of (7-bromonaphthalen-2-yl)methanol (3, 40.0 g, 0.17 mol, 1.0 eq.) in CCl₄ (400 mL) was added PBr₃ (16.1 g, 0.06 mol, 0.35 eq.) at 60° C. After addition, the reaction mixture was stirred at 60° C. for 3 h. After completion, the reaction mixture was concentrated under reduced pressure to give a solid. The solid was triturated with PE/EA=8/1 (400 mL) to afford 2-bromo-7-(bromomethyl)naphthalene (4, 48.5 g, 0.16 mol, 96%) as a white solid. ¹H NMR (400 MHz, DMSO-d₆) δ 8.26 (d, J=1.7 Hz, 1H), 8.08-7.90 (m, 3H), 7.75-7.56 (m, 2H), 4.93 (s, 2H).

### Step D: Diethyl ((7-bromonaphthalen-2-yl)methyl)phosphonate

A solution of 2-bromo-7-(bromomethyl)naphthalene (4, 48.5 g, 0.16 mol, 1.0 eq.) in triethyl phosphite (100 mL) was stirred at 100° C. overnight under nitrogen atmosphere. After completion, the reaction mixture was concentrated under reduced pressure to remove triethyl phosphite. The residue was triturated with IPA (100 mL) to afford diethyl ((7-bromonaphthalen-2-yl)methyl)phosphonate (5, 40.0 g, 0.11 mol, 69%) as a white solid. LCMS (ESI): m/z=357.1 [M+H]⁺. ¹H NMR (400 MHz, CDCl₃) δ 7.95 (s, 1H), 7.76 (d, J=8.4 Hz, 1H), 7.69-7.65 (m, 2H), 7.57-7.39 (m, 2H), 4.13-3.90 (m, 4H), 3.31 (d, J=21.8 Hz, 2H), 1.24 (t, J=7.1 Hz, 6H).

### Step E: 7-((diethoxyphosphoryl)methyl)-2-naphthoic acid

To a solution of diethyl ((7-bromonaphthalen-2-yl)methyl)phosphonate (5, 10.0 g, 27.9 mmol, 1.0 eq.), oxalic acid (10.9 g, 122.0 mmol, 4.4 eq.), Ac₂O (10.5 g, 103.0 mmol, 3.7 eq.) and DIEA (19.3 g, 150.0 mmol, 5.4 eq.) in DMF (60.0 mL) was added Pd(OAc)₂ (625 mg, 2.79 mmol, 0.1 eq.) and PPh₃ (2.2 g, 8.37 mmol, 0.3 eq.). The resulting mixture was stirred at 100° C. overnight under nitrogen atmosphere. After completion, the reaction mixture was cooled to room temperature and acidified with 1 N HCl aqueous solution until the pH was adjusted to pH=3. The mixture was filtered. The filtrate was diluted with H₂O (40 mL) and extracted with EtOAc (200 mL×3). The organic layers were combined, washed with brine (50 mL), dried over anhydrous Na₂SO₄, and concentrated under reduced pressure to afford 7-((diethoxyphosphoryl)methyl)-2-naphthoic acid (6, 8.0 g, 24.8 mmol, 89%) as a yellow solid. LCMS (ESI): m/z=323.2 [M+H]⁺.

### Synthesis of ((7-((allyloxy)carbonyl)naphthalen-2-yl)methyl)phosphonic acid and ((7-((allyloxy)carbonyl)naphthalen-2-yl)fluoromethyl)phosphonic acid

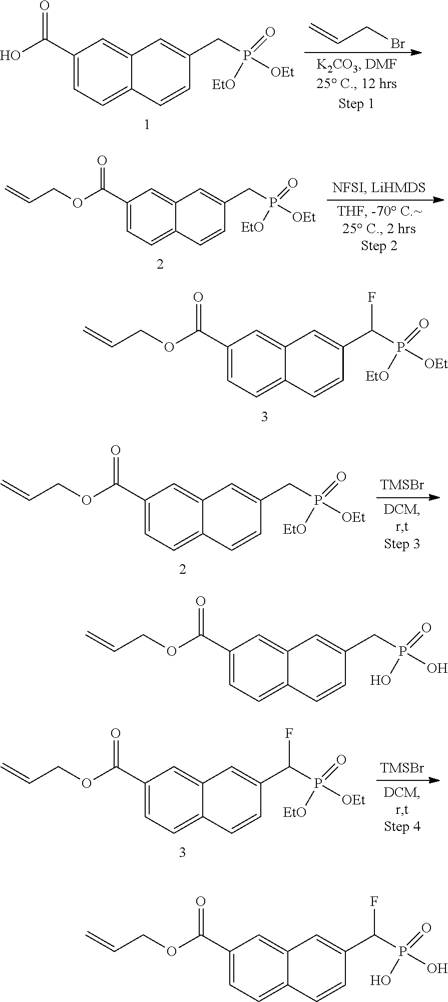

### Step 1: Allyl 7-((diethoxyphosphoryl)methyl)-2-naphthoate

Four batches of the reactions were carried out in parallel.

To a solution of 7-((diethoxyphosphoryl)methyl)-2-naphthoic acid (Compound 1, 150 g, 465 mmol, 1.00 eq) and K₂CO₃ (129 g, 931 mmol, 2.00 eq) in DMF (1.50 L) was added allyl bromide (67.6 g, 559 mmol, 1.20 eq) at 25° C. After addition, the reaction mixture was stirred at 25° C. for 12 h, at which time LCMS indicated reaction was complete. Four batches of reaction mixture were combined for work-up. The reaction mixture was poured into water (18.0 L) and extracted with ethyl acetate (6.00 L×2). The organic layers were combined, washed with water (6.00 L) and brine (6.00 L), dried over anhydrous Na₂SO₄, and concentrated under reduced pressure to give a crude product. The crude product was purified by silica gel column chromatography (Petroleum ether:Ethyl acetate=50:1 to 0:1, Petroleum ether:Ethyl acetate=0:1, R_{f}=0.50) to give allyl 7-((diethoxyphosphoryl)methyl)-2-naphthoate (Compound 2 568 g, 1.54 mol, 82.7% yield) as a light-yellow solid. LCMS (ESI): m/z=363.1 [M+H]⁺. ¹H NMR (400 MHz, CDCl₃) δ 8.59 (s, 1H), 8.07-8.06 (d, J=7.60 Hz, 1H), 7.88-7.84 (m, 3H), 7.58-7.56 (m, 1H), 6.13-6.06 (m, 1H), 5.49-5.44 (m, 1H), 5.35-5.32 (m, 1H), 4.91-4.89 (m, 2H), 4.06-4.02 (m, 4H), 3.36-3.31 (d, J=22.0 Hz 1H), 1.27-1.23 (m, 6H).

### Step 2: Allyl 7-((diethoxyphosphoryl)fluoromethyl)-2-naphthoate

Eight batches of the reactions were carried out in parallel.

To a solution of allyl 7-((diethoxyphosphoryl)methyl)-2-naphthoate (Compound 2, 100 g, 276 mmol, 1.00 eq) in THF (1.50 L) was added NFSI (87.0 g, 276 mmol, 1.00 eq) at 25° C. slowly. The mixture was cooled to −70° C. A solution of LiHMDS (1.00 M, 276 mL, 1.00 eq) was added to the above mixture drop-wise under N₂. After addition, the reaction mixture was stirred at −70° C. for 2 h under N₂. The reaction mixture was poured into saturated NH₄Cl aqueous solution (2.00 L) slowly at 0° C. The mixture was extracted with ethyl acetate (1.00 L*2). The organic layers were combined, washed with brine (4.00 L), dried over anhydrous Na₂SO₄ and concentrated under reduced pressure to give a residue. The residue was diluted with ethyl acetate (4.00 L) and filtered; the filtrate was concentrated under reduced pressure to give a crude yellow oil (779 g). The product was purified by silica gel column chromatography (Petroleum ether:Ethyl acetate=50:1 to 0:1) to give allyl 7-((diethoxyphosphoryl)fluoromethyl)-2-naphthoate (Compound 3, 418 g, 1.07 mol, 52.3% yield, 97.4% purity) as yellow oil. LCMS (ESI): m/z=381.1 [M+H]⁺. ¹H NMR (400 MHz, DMSO) δ 8.71 (s, 1H), 8.23 (s, 1H), 8.10-8.04 (m, 3H), 7.74-7.72 (d, J=8.80 Hz, 1H), 6.39-6.26 (m, 1H), 6.14-6.10 (m, 1H), 5.49-5.44 (m, 1H), 5.33-5.30 (m, 1H), 4.89-4.87 (m, 2H), 4.07-4.01 (m, 4H), 1.22-1.15 (m, 6H).

### Step 3: ((7-((allyloxy)carbonyl)naphthalen-2-yl)methyl)phosphonic acid

To a solution of allyl 7-((diethoxyphosphoryl)methyl)-2-naphthoate (Compound 2, 4 g, 11.0 mmol, 1.0 eq.) in DCM (40 mL) was added TMSBr (8 mL) at room temperature. After addition, the reaction mixture was stirred at room temperature for 12 h. After completion, the reaction mixture was poured into H₂O (30 mL) and the suspension was filtered. The filter cake was dried under vacuum to afford ((7-((allyloxy)carbonyl)naphthalen-2-yl)methyl)phosphonic acid (3.0 g, 9.79 mmol, 89%) as a yellow solid. LCMS (ESI): m/z=307 [M+H]⁺.

¹H NMR (400 MHz, DMSO-d₆) δ 8.56 (s, 1H), 8.04-7.87 (m, 4H), 7.64-7.56 (m, 1H), 6.21-5.96 (m, 1H), 5.53-5.23 (m, 2H), 4.93-4.82 (m, 2H), 3.17 (d, J=21.5 Hz, 2H).

### Step 4: ((7-((allyloxy)carbonyl)naphthalen-2-yl)fluoromethyl)phosphonic acid

To a solution of allyl 7-((diethoxyphosphoryl)fluoromethyl)-2-naphthoate (Compound 3, 27 g, 71.1 mmol, 1.0 eq.) in DCM (300 mL) was added TMSBr (50 mL) at room temperature. After addition, the reaction mixture was stirred at room temperature for 12 h. After completion, the reaction mixture was concentrated under reduced pressure. The residue was purified by Biotage® C18 column chromatography to afford ((7-((allyloxy)carbonyl)naphthalen-2-yl)fluoromethyl)phosphonic acid (17.6 g, 54.2 mmol, 76%) as a white solid.

¹H NMR (400 MHz, DMSO-d₆) δ 8.67 (s, 1H), 8.16 (s, 1H), 8.10-7.96 (m, 3H), 7.73 (d, J=8.5 Hz, 1H), 6.18-6.04 (m, 1H), 5.89 (dd, J=44.5, 8.6 Hz, 1H), 5.52-5.42 (m, 1H), 5.36-5.26 (m, 1H), 4.88 (d, J=5.4 Hz, 2H). LCMS (ESI): m/z=325.1 [M+H]⁺.

### Chiral separation of allyl 7-((diethoxyphosphoryl)fluoromethyl)-2-naphthoate

Rac-allyl 7-((diethoxyphosphoryl)fluoromethyl)-2-naphthoate (617 g, 1.62 mol, 1.00 eq) was purified by SFC to give allyl (S)-7-((diethoxyphosphoryl)fluoromethyl)-2-naphthoate (Peak 1) and allyl (R)-7-((diethoxyphosphoryl)fluoromethyl)-2-naphthoate (Peak 2).

Preparative SFC method: Instrument: Waters 350 Preparative SFC. Column: REGIS (S,S) WHELK-O1, 250×50 mm I.D., 10 μm. Mobile phase: A for CO₂ and B for MeOH (Neu). Gradient: B 15% Flow rate: 220 g/min. Back pressure: 100 bar. Column temperature: 35° C. Wavelength: 254 nm. Cycle-time: 7.3 min.

Analytical SFC method: Column: Kromasil (S,S) WHELK-O1, 50×4.6 mm I.D., 3.5 μm. Mobile phase: A for CO₂ and B for MEOH (0.05% DEA). Gradient: B 5 to 40% Flow rate: 3 mL/min. Back pressure: 100 bar. Column temperature: 35° C. Wavelength: 220 nm.

allyl (S)-7-((diethoxyphosphoryl)fluoromethyl)-2-naphthoate (Peak 1, 286 g, 745 mmol, 45.9% yield, >99% ee, RT=1.31 min) was obtained as yellow oil. LCMS (ESI): m/z=381.1 [M+H]⁺.

allyl (R)-7-((diethoxyphosphoryl)fluoromethyl)-2-naphthoate (Peak 2, 288 g, 748 mmol, 46.1% yield, >99% ee, RT=1.52 min) was obtained as yellow oil. LCMS (ESI): m/z=381.1 [M+H]⁺.

Assignment of absolute stereochemical configuration was made by comparison of experimental vibrational circular dichroism (VCD) spectra with theoretical VCD spectra obtained from DFT calculations.

The following intermediates in Table 56 were prepared using the method described above in step 4 for the preparation of ((7-((allyloxy)carbonyl)naphthalen-2-yl)fluoromethyl)phosphonic acid and utilizing the appropriate starting materials and modifications.

TABLE 56

| Name | Structure | LCMS |
|---|---|---|
| (S)-((7-((allyloxy)carbonyl)naphthalen-2- yl)fluoromethyl)phosphonic acid | 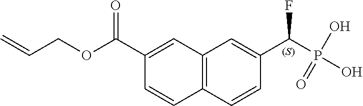 | 325.1 [M + H]⁺ |
| (R)-((7-((allyloxy)carbonyl)naphthalen-2- yl)fluoromethyl)phosphonic acid | 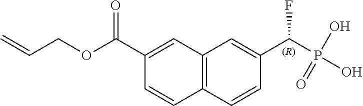 | 325.1 [M + H]⁺ |

### Synthesis of ((2-((allyloxy)carbonyl)benzo[b]thiophen-5-yl)methyl)phosphonic acid and ((2-((allyloxy)carbonyl)benzo[b]thiophen-5-yl)fluoromethyl)phosphonic acid

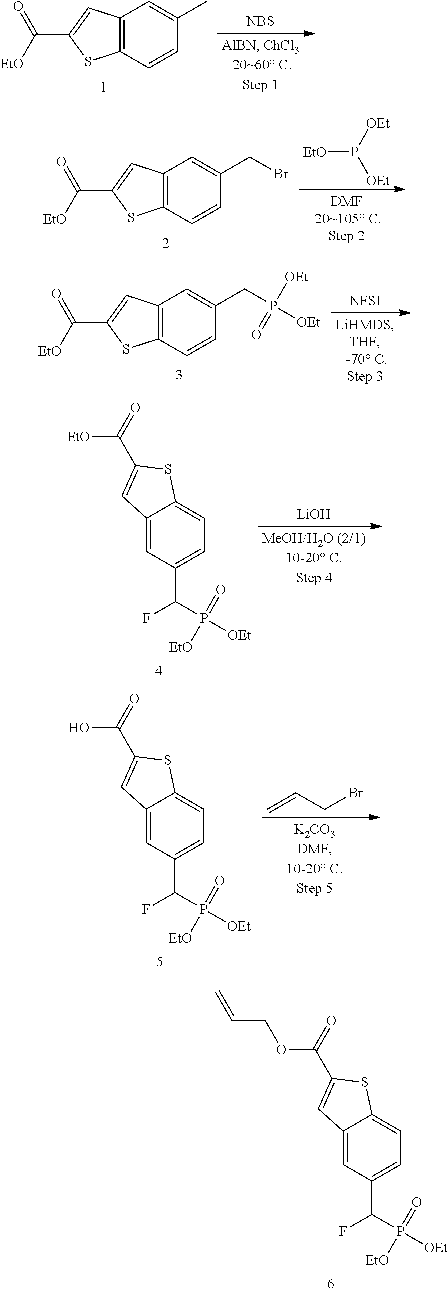

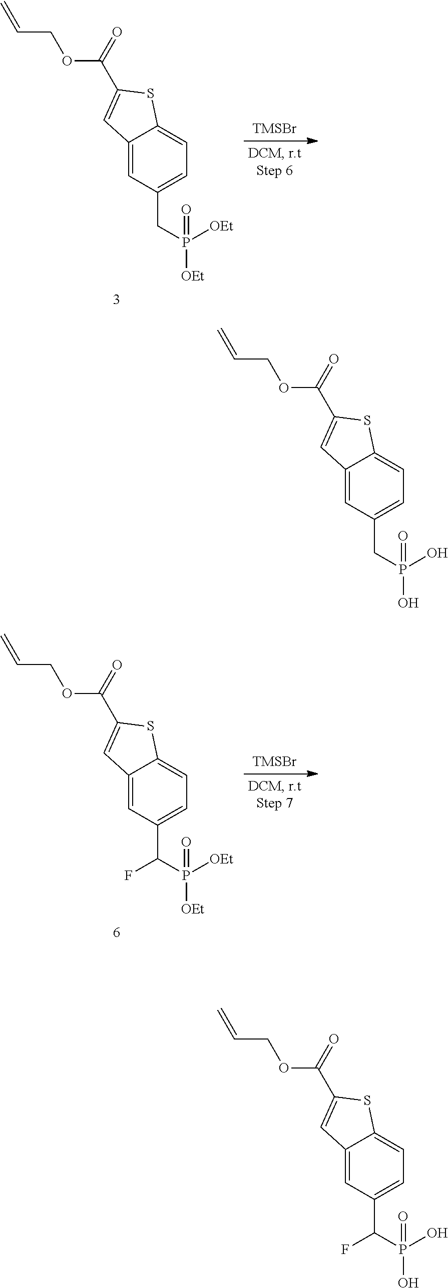 [SMILES: "TMSBr" ; C=CCOC(=O)c1cc2cc(CP(=O)(OCC)OCC)ccc2s1 ; "TMSBr" ; C=CCOC(=O)c1cc2cc(CP(=O)(O)O)ccc2s1 ; C=CCOC(=O)c1cc2cc(C(F)P(=O)(OCC)OCC)ccc2s1 ; C=CCOC(=O)c1cc2cc(C(F)P(=O)(O)O)ccc2s1]

Ethyl 5-methylbenzo[b]thiophene-2-carboxylate was prepared according to the procedure described in WO 2016/100184.

### Step 1: Ethyl 5-(bromomethyl)benzo[b]thiophene-2-carboxylate

To a solution of ethyl 5-methylbenzo[b]thiophene-2-carboxylate (1, 7.3 kg, 33.1 mol, 1.0 eq.) in CHCl₃ (58 L) stirred at 20° C. was added AIBN (544 g, 3.31 mol, 0.10 eq.) and NBS (6.19 kg, 34.8 mol, 1.05 eq.). The mixture was heated from 30° C. to 50° C. over 4 h and was then heated to 60° C. and stirred for 12 hours. After completion, the reaction mixture was cooled to 10° C. and 15% Na₂SO₃ (20 L) was added. The organic layers were washed with H₂O (20 L*2), dried over Na₂SO₄, and concentrated under reduced pressure at 45° C. to afford ethyl 5-(bromomethyl)benzo[b]thiophene-2-carboxylate (2, 9.50 kg, 23.5 mol, 70.9% yield, 74.0% purity) as a yellow solid. LCMS (ESI): m/z=298.9 [M+H]⁺.

¹H NMR (400 MHz, CDCl₃) δ 8.034 (s, 1H), 7.89-7.84 (m, 2H), 7.50 (d, J=9.6 Hz, 1H), 4.64 (s, 2H), 4.45-4.40 (m, 2H), 1.45-1.41 (m, 3H).

### Step 2: Ethyl 5-((diethoxyphosphoryl)methyl)benzo[b]thiophene-2-carboxylate

To a solution of ethyl 5-(bromomethyl)benzo[b]thiophene-2-carboxylate (2, 9.50 kg, 31.8 mol, 1.0 eq.) in DMF (28.5 L) stirred at 20° C. was added triethyl phosphite (5.8 kg, 34.9 mol, 1.1 equiv). The mixture was heated to 100° C. and stirred for 5 h. After completion, the reaction mixture was cooled to 15° C., poured into H₂O (50.0 L), and extracted with EtOAc (20 L*2). The combined organics were washed with H₂O (20 L*2) and brine (10 L), dried over Na₂SO₄, and concentrated under reduced pressure at 45° C. to give a residue. Crude residue was purified by silica gel chromatography (Petroleum ether/Ethyl acetate=100/1 to 1/1, Petroleum ether/Ethyl acetate=0/1) to afford ethyl 5-((diethoxyphosphoryl)methyl)benzo[b]thiophene-2-carboxylate (3, 5.24 kg, 14 mol, 44.4% yield) as yellow solid. LCMS (ESI): m/z=356.9 [M+H]⁺. ¹H NMR (400 MHz, CDCl₃) δ 8.02 (s, 1H), 7.82-7.80 (m, 2H), 7.41 (d, J=2.0 Hz, 1H), 4.44-4.41 (m, 2H), 4.05-4.01 (m, 4H), 3.27 (d, J=21.6 Hz, 2H), 1.46-1.42 (m, 3H), 1.27-1.23 (m, 6H).

### Step 3: Ethyl 5-((diethoxyphosphoryl)fluoromethyl)benzo[b]thiophene-2-carboxylate

Three batches were carried out in parallel.

To a solution of ethyl 5-((diethoxyphosphoryl)methyl)benzo[b]thiophene-2-carboxylate (3, 250 g, 702 mmol, 1.00 eq) and N-(benzenesulfonyl)-N-fluorobenzenesulfonamide (221 g, 702 mmol, 1.00 eq) in THF (2.50 L) was added dropwise LiHMDS (1 M, 702 mL, 1.00 eq) at −70° C. under N₂. The mixture was stirred at −70° C. for 3 h. Following completion, the reaction mixture was poured into saturated NH₄Cl aqueous solution (5.00 L) slowly at 0° C. and the mixture was stirred at 0° C. for 0.5 hr. Then three batches were combined to workup. The mixture was extracted with ethyl acetate (5.00 L*3). The organic layers were combined, washed with brine (5.00 L), dried over anhydrous Na₂SO₄ and concentrated under reduced pressure. The residue was purified by column chromatography (SiO₂, petroleum ether/ethyl acetate=1/0-3/1, R_{f}=0.30, petroleum ether/ethyl acetate=1/1) to give ethyl 5-((diethoxyphosphoryl)fluoromethyl)benzo[b]thiophene-2-carboxylate (4, 357 g, 928 mmol, 44.1% yield) as yellow oil. LCMS (ESI): m/z=375.0 [M+H]⁺. ¹H NMR (400 MHz, CDCl₃) δ 8.07 (s, 1H), 8.00 (s, 1H), 7.90 (d, J=8.4 Hz, 1H), 7.59 (d, J=8.8 Hz, 1H), 5.88-5.75 (m, 1H), 4.45-4.40 (m, 2H), 4.15-4.05 (m, 4H), 1.45-1.41 (m 3H), 1.31-1.28 (m, 6H).

### Step 4: 5-((diethoxyphosphoryl)fluoromethyl)benzo[b]thiophene-2-carboxylic acid

To a solution of ethyl 5-((diethoxyphosphoryl)fluoromethyl)benzo[b]thiophene-2-carboxylate (4, 252 g, 673 mmol, 1.00 eq) in MeOH (1.80 L) was added H₂O (760 mL) and LiOH·H₂O (56.5 g, 1.35 mol, 2.00 eq) at 10-20° C. under N₂. The mixture was stirred at 10-20° C. for 1 hr. TLC (petroleum ether/ethyl acetate=1/1) showed that compound 4 was consumed (R_{f}=0.30) and desired spot (R_{f}=0.10) was formed. The reaction mixture was quenched by H₂O (2.50 L) and then adjusted pH to 3-4 with HCl (aq.1M). The mixture was extracted with dichloromethane (2.50 L*3). The combined organic layers were dried over anhydrous Na₂SO₄ and concentrated under reduced pressure to give 5-((diethoxyphosphoryl)fluoromethyl)benzo[b]thiophene-2-carboxylic acid (5, 222 g, 626 mmol, 93.0% yield) as a white solid.

### Step 5: Allyl 5-((diethoxyphosphoryl)fluoromethyl)benzo[b]thiophene-2-carboxylate

Two batches were carried out in parallel.

To a solution of 5-((diethoxyphosphoryl)fluoromethyl)benzo[b]thiophene-2-carboxylic acid (5, 178 g, 514 mmol, 1.00 eq) in DMF (1.78 L) was added K₂CO₃ (142 g, 1.03 mol, 2.00 eq) and allyl bromide (68.4 g, 565 mmol, 1.10 eq) at 10-20° C. The mixture was stirred at 10-20° C. for 12 h. TLC (petroleum ether/ethyl acetate=0/1) showed that compound 5 was consumed (R_{f}=0.60) and a new spot (R_{f}=0.70) was formed. The reaction mixture was diluted with H₂O (6.00 L), extracted with ethyl acetate (2.00 L*3). The organic layers were combined. The mixture was washed with brine (2.00 L) and NH₄Cl (2.00 L), dried over anhydrous Na₂SO₄ and concentrated under reduced pressure to give allyl 5-((diethoxyphosphoryl)fluoromethyl)benzo[b]thiophene-2-carboxylate (6, 388 g, 985 mmol, 95.8% yield) as yellow oil.

### Step 6: ((2-((allyloxy)carbonyl)benzo[b]thiophen-5-yl)methyl)phosphonic acid

To a solution of allyl 5-((diethoxyphosphoryl)methyl)benzo[b]thiophene-2-carboxylate (3, 50 g, 136 mmol, 1.0 eq.) in DCM (5 L) was added TMSBr (411 g, 2.71 mol, 20.0 eq.) dropwise at 0° C. After addition, the reaction mixture was allowed to warm to room temperature and stirred for an additional 12 h. The reaction progress was monitored by LCMS. After completion, the reaction mixture was concentrated under reduced pressure and water was added. The resulting mixture was filtered and the filter cake was washed with water (2 L), then dried in vacuum to afford ((2-((allyloxy)carbonyl)benzo[b]thiophen-5-yl)methyl)phosphonic acid (40.3 g, 129 mmol, 95%) as a yellow solid. ¹H NMR (400 MHz, DMSO-d₆) δ 8.20 (s, 1H), 7.96 (d, J=8.4 Hz, 1H), 7.87 (s, 1H), 7.43 (d, J=8.4 Hz, 1H), 6.10-5.98 (m, 1H), 5.46-5.37 (m, 1H), 5.33-5.24 (m, 1H), 4.86-4.77 (m, 2H), 3.08 (d, J=21.2 Hz, 2H). LCMS (ESI) m/z=313.1 [M+H]⁺.

### Step 7: ((2-((allyloxy)carbonyl)benzo[b]thiophen-5-yl)fluoromethyl)phosphonic acid

To a solution of allyl 5-((diethoxyphosphoryl)fluoromethyl)benzo[b]thiophene-2-carboxylate (6, 5.2 g, 13.5 mmol, 1.0 eq.) in DCM (500 mL) was added TMSBr (41.1 g, 270 mmol, 20.0 eq.) dropwise at 0° C. After the addition, the reaction mixture was allowed to warm to room temperature and stirred for an additional 12 h. The reaction progress was monitored by LCMS. After completion, the reaction mixture was concentrated under reduced pressure and water was added. The resulting mixture was filtered and the filter cake was washed with water (200 mL), then dried in vacuum to afford ((2-((allyloxy)carbonyl)benzo[b]thiophen-5-yl)fluoromethyl)phosphonic acid (3.1 g, 9.37 mmol, 69%) as a yellow solid. ¹H NMR (400 MHz, DMSO-d₆) δ 8.32 (s, 1H), 8.14-8.05 (m, 2H), 7.59 (d, J=8.4 Hz, 1H), 6.12-5.99 (m, 1H), 5.84 (dd, J=44.3, 8.2 Hz, 1H), 5.49-5.40 (m, 1H), 5.35-5.27 (m, 1H), 4.88-4.81 (m, 2H). LCMS (ESI): m/z=329.1 [M−H]⁻.

### Chiral separation of allyl 5-((diethoxyphosphoryl)fluoromethyl)benzo[b]thiophene-2-carboxylate

Rac-allyl 5-((diethoxyphosphoryl)fluoromethyl)benzo[b]thiophene-2-carboxylate (617 g, 1.62 mol, 1.00 eq) was purified by SFC to give allyl (S)-5-((diethoxyphosphoryl)fluoromethyl)benzo[b]thiophene-2-carboxylate (Peak 1) and allyl (R)-5-((diethoxyphosphoryl)fluoromethyl)benzo[b]thiophene-2-carboxylate (Peak 2).

Preparative SFC method: Instrument: Waters 350 Preparative SFC. Column: REGIS (S,S) WHELK-O1, 250×50 mm I.D., 10 μm. Mobile phase: A for CO₂ and B for MEOH (Neu). Gradient: B 30%. Flow rate: 220 g/min. Back pressure: 100 bar. Column temperature: 35° C. Wavelength: 220 nm. Cycle-time: 3.3 min.
Analytical SFC method: Column: Kromasil (S,S) WHELK-O1, 50×4.6 mm I.D., 3.5 μm.
Mobile phase: A for CO₂ and B for MEOH (0.05% DEA). Gradient: B 5 to 40% Flow rate: 3 mL/min. Back pressure: 100 bar. Column temperature: 35° C. Wavelength: 220 nm.

allyl (S)-5-((diethoxyphosphoryl)fluoromethyl)benzo[b]thiophene-2-carboxylate (Peak 1, 267 g, 685 mmol, 39.7% yield, >99% ee, RT=1.36 min) was obtained as yellow oil. LCMS (ESI): m/z=387.1 [M+H]⁺.

allyl (R)-5-((diethoxyphosphoryl)fluoromethyl)benzo[b]thiophene-2-carboxylate (Peak 2, 270 g, 676 mmol, 39.2% yield, >99% ee, RT=1.55 min) was obtained as yellow oil. LCMS (ESI): m/z=387.1 [M+H]⁺.

Assignment of absolute stereochemical configuration was made by comparison of experimental vibrational circular dichroism (VCD) spectra with theoretical VCD spectra obtained from DFT calculations.

The following intermediates in Table 57 were prepared using the method described above in step 7 for the preparation of ((2-((allyloxy)carbonyl)benzo[b]thiophen-5-yl)fluoromethyl)phosphonic acid and utilizing the appropriate starting materials and modifications.

TABLE 57

| Name | Structure | LCMS |
|---|---|---|
| (S)-((2- ((allyloxy)carbonyl)benzo[b]thiophen-5- yl)fluoromethyl)phosphonic acid | 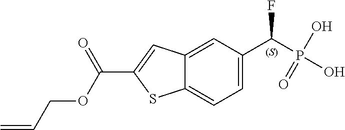 | 329 [M − H]⁻ |
| (R)-((2- ((allyloxy)carbonyl)benzo[b]thiophen-5- yl)fluoromethyl)phosphonic acid | 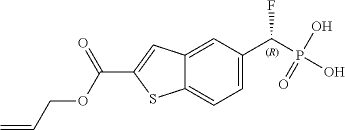 | 329 [M − H]⁻ |

### Synthesis of 5-(((2,6-dimethylphenoxy)(((S)-1-oxo-1-propoxypropan-2-yl)amino)phosphoryl)methyl)benzo[b]thiophene-2-carboxylic acid

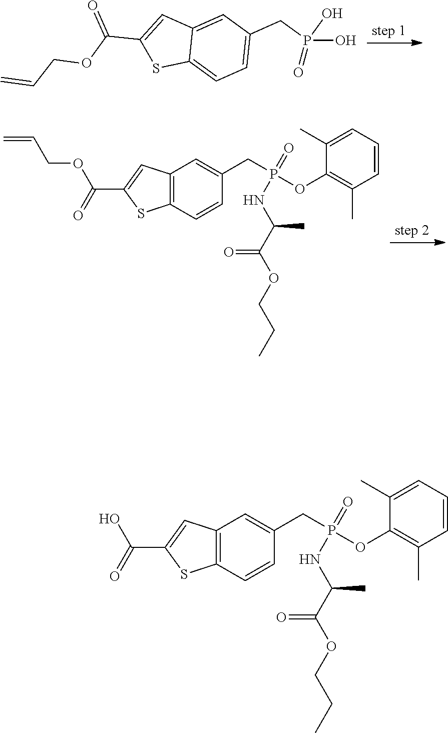 [SMILES: C=CCOC(=O)c1cc2cc(CP(=O)(O)O)ccc2s1 ; C=CCOC(=O)c1cc2cc(CP(=O)(N[C@@H](C)C(=O)OCCC)Oc3c(C)cccc3C)ccc2s1 ; CCCOC(=O)[C@H](C)NP(=O)(Cc1ccc2sc(C(=O)O)cc2c1)Oc1c(C)cccc1C]

### Step 1: Preparation of Allyl 5-(((2,6-dimethylphenoxy)(((S)-1-oxo-1-propoxypropan-2-yl)amino)phosphoryl)methyl)benzo[b]thiophene-2-carboxylate

To a suspension of ((2-((allyloxy)carbonyl)benzo[b]thiophen-5-yl)methyl)phosphonic acid (0.150 g, 480 μmol, 1 eq) and 1 drop of DMF (cat.) in DCM (4 mL) at 0° C. was added oxalyl chloride (122 μL, 1.44 mmol, 3 eq) and the mixture was stirred for 2 days at room temperature. The reaction was concentrated under reduced pressure and then dried under high vacuum for 20 min. The crude residue was diluted in toluene (5 mL). A solution of 2,6-dimethylphenol (58.6 mg, 480 μmol, 1 eq) and triethylamine (333 μL, 2.40 mmol, 5 eq) in toluene (2 mL, dried with sodium sulfate) was added to the mixture and stirred at 90° C. for 3 h. Propyl (2S)-2-aminopropanoate hydrochloride (80.4 mg, 480 μmol, 1 eq) was added at once at 90° C. and the reaction was stirred at 90° C. for 2 h. The reaction was cooled down to room temperature and quenched with 2 drops of water and toluene was removed under reduced pressure. The product was purified by reverse phase chromatography on a 100 g C₁₈ cartridge eluting with a gradient of 5-100% acetonitrile in water (with 0.1% formic acid) to afford allyl 5-(((2,6-dimethylphenoxy)(((S)-1-oxo-1-propoxypropan-2-yl)amino)phosphoryl)methyl)benzo[b]thiophene-2-carboxylate (50.0 mg, 19.6%) as a pale yellow oil. LCMS (ESI) m/z=530.4 [M+H]⁺.

The following intermediates in Table 58 were prepared using the method described above in step 1 for the preparation of allyl 5-(((2,6-dimethylphenoxy)(((S)-1-oxo-1-propoxypropan-2-yl)amino)phosphoryl)methyl)benzo[b]thiophene-2-carboxylate and utilizing the appropriate starting materials and modifications.

TABLE 58

| Name | Structure | LCMS | NMR |
|---|---|---|---|
| allyl 5-((((2-methyl-3- oxo-3- propoxypropyl)amino) (phenoxy)phosphoryl) methyl)benzo[b] thiophene-2- carboxylate | 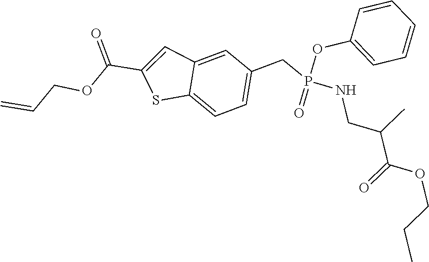 [SMILES: C=CCOC(=O)c1cc2cc(CP(=O)(NCC(C)C(=O)OCCC)Oc3ccccc3)ccc2s1] | 516.4 [M + H]⁺ | ¹H NMR (400 MHz, CDCl₃) δ 8.07 − 8.02 (m, 1H), 7.88 − 7.80 (m, 2H), 7.50 − 7.43 (m, 1H), 7.33 − 7.27 (m, 2H), 7.19 − 7.10 (m, 3H), 6.11 − 5.99 (m, 1H), 5.49 − 5.41 (m, 1H), 5.36 − 5.30 (m, 1H), 4.89 − 4.83 (m, 2H), 4.03 − 3.85 (m, 2H), 3.47 − 3.36 (m, 2H), 3.14 − 2.95 (m, 3H), 2.43 − 2.31 (m, 1H), 1.64 − 1.51 (m, 2H), 1.01 (d, J = 7.3 |
| | | | Hz, 3H), 0.92 − 0.83 |
| | | | (m, 3H) |
| butyl (2S)-1-(((2- ((allyloxy)carbonyl) benzo[b]thiophen-5- yl)methyl)(phenoxy) phosphoryl)-4,4- difluoropyrrolidine- 2-carboxylate | 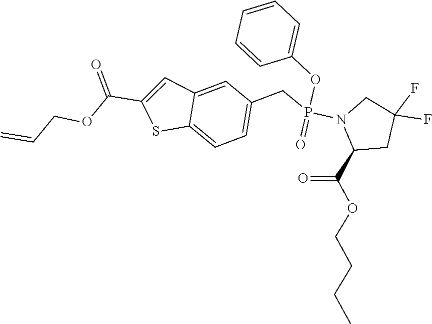 [SMILES: C=CCOC(=O)c1cc2cc(CP(=O)(Oc3ccccc3)N3CC(F)(F)C[C@H]3C(=O)OCCCC)ccc2s1] | 578.4 [M + H]⁺ | ¹H NMR (400 MHz, CDCl₃) δ 8.10 − 8.02 (m, 1H), 8.00 − 7.89 (m, 1H), 7.88 − 7.80 (m, 1H), 7.63 − 7.50 (m, 1H), 7.37 − 7.10 (m, 5H), 6.14 − 5.98 (m, 1H), 5.49 − 5.42 (m, 1H), 5.39 − 5.28 (m, 1H), 4.89 − 4.83 (m, 2H), 4.73 − 4.67 (m, 0.4H), 4.30 − 4.23 (m, 0.6H), 4.23 − 4.02 (m, 2H), 3.82 − 3.59 (m, 1H), 3.58 − 3.41 (m, 2H), 3.31 − 3.04 (m, 1H), 2.54 − 2.20 (m, 2H), 1.70 − 1.48 (m, 2H), 1.45 − 1.15 (m, 2H), 0.98 − 0.88 (m, 3H) |
| allyl 5-(((((S)-1-oxo-1- propoxybutan-2- yl)amino)(phenoxy) phosphoryl)methyl) benzo[b]thiophene-2- carboxylate | 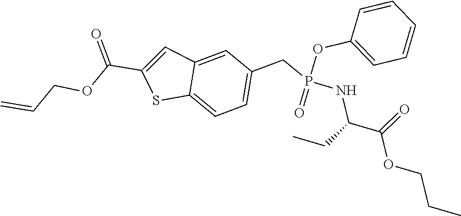 [SMILES: C=CCOC(=O)c1cc2cc(CP(=O)(N[C@@H](CC)C(=O)OCCC)Oc3ccccc3)ccc2s1] | 516.2 [M + H]⁺ | ¹H NMR (400 MHz, CDCl₃) δ 8.09 − 8.00 (m, 1H), 7.93 − 7.81 (m, 2H), 7.56 − 7.44 (m, 1H), 7.33 − 7.27 (m, 2H), 7.19 − 7.04 (m, 3H), 6.12 − 6.00 (m, 1H), 5.49 − 5.42 (m, 1H), 5.37 − 5.30 (m, 1H), 4.89 − 4.82 (m, 2H), 4.05 − 3.87 (m, 3H), 3.51 − 3.39 (m, 2H), 3.33 − 3.07 (m, 1H), 1.63 − 1.46 (m, |
| | | | 4H), 0.93 − 0.83 (m, |
| | | | 3H), 0.78 − 0.59 (m, 3H) |
| allyl 5-((((4-oxo-4- propoxybutan-2- yl)amino)(phenoxy) phosphoryl)methyl) benzo[b]thiophene-2- carboxylate | 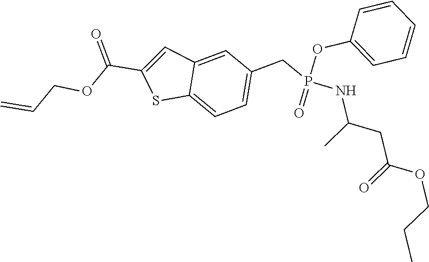 [SMILES: C=CCOC(=O)c1cc2cc(CP(=O)(NC(C)CC(=O)OCCC)Oc3ccccc3)ccc2s1] | 516.2 [M + H]⁺ | ¹H NMR (400 MHz, CDCl₃) δ 8.07 − 8.02 (m, 1H), 7.90 − 7.78 (m, 2H), 7.50 − 7.44 (m, 1H), 7.34 − 7.27 (m, 2H), 7.21 − 7.08 (m, 3H), 6.13 − 5.99 (m, 1H), 5.49 −5.41 (m, 1H), 5.36 − 5.30 (m, 1H), 4.89 (m, 2H) 4.03 3.86 (m, 2H), 3.80 − 3.64 (m, 1H), 3.48 − 3.36 (m, 2H), 3.33 − 3.12 (m, 1H), 2.34 − 2.14 (m, 2H), 1.67 − 1.50 (m, 2H), 1.10 − 0.97 (m, 3H), 0.95 − |
| | | | 0.85 (m, 3H) |
| butyl (((2- ((allyloxy)carbonyl) benzo[b]thiophen-5- yl)methyl)(phenoxy) phosphoryl)-L-prolinate | 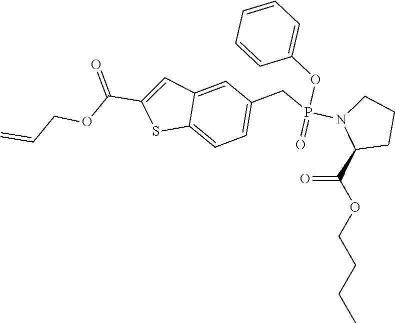 [SMILES: C=CCOC(=O)c1cc2cc(CP(=O)(Oc3ccccc3)N3CCC[C@H]3C(=O)OCCCC)ccc2s1] | 542.3 [M + H]⁺ | |
| allyl 5-(((((S)-1- butoxy-1-oxopropan- 2- yl)amino)(naphthalen- 1- yloxy)phosphoryl) methyl)benzo [b]thiophene- 2-carboxylate | 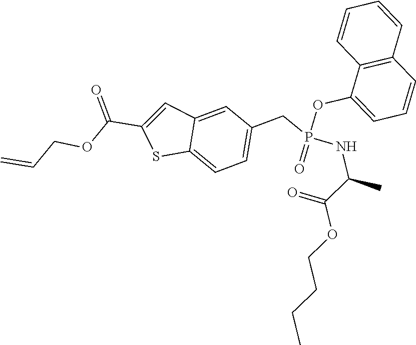 [SMILES: C=CCOC(=O)c1cc2cc(CP(=O)(N[C@@H](C)C(=O)OCCCC)Oc3cccc4ccccc34)ccc2s1] | 566.1 [M + H]⁺ | |
| allyl 5-(((((S)-3- methyl-1-oxo-1- propoxybutan-2- yl)amino)(phenoxy) phosphoryl)methyl) benzo[b]thiophene-2- carboxylate | 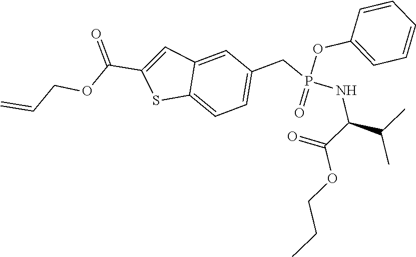 [SMILES: C=CCOC(=O)c1cc2cc(CP(=O)(N[C@H](C(=O)OCCC)C(C)C)Oc3ccccc3)ccc2s1] | | ¹H NMR (400 MHz, CDCl3) δ 8.05 (d, J = 6.0 Hz, 1H), 7.92 − 7.80 (m, 2H), 7.55 − 7.45 (m, 1H), 7.30 − 7.27 (m, 1H), 7.27 − 7.24 (m, 1H), 7.15 − 7.08 (m, 3H), 6.13 − 5.99 (m, 1H), 5.45 (td, J = 1.2, 17.2 Hz, 1H), 5.36 − 5.31 (m, 1H), 5.31 (s, 1H), 4.86 (d, J = 5.6 Hz, 2H), 4.03 − 3.90 (m, 1H), 3.86 − 3.75 (m, 1H), 3.50 − 3.39 (m, 2H), 3.21 − 3.03 (m, |
| | | | 1H), 1.94 − 1.83 (m, |
| | | | 1H), 1.59 − 1.50 (m, |
| | | | 2H), 0.91 − 0.86 (m, |
| | | | 3H), 0.85 − 0.78 (m, |
| | | | 3H), 0.67 (dd, J = 4.0, |
| | | | 6.8 Hz, 3H) |
| allyl 5-(((((S)-1-((R)- sec-butoxy)-1- oxopropan-2- yl)amino)(phenoxy) phosphoryl)methyl) benzo[b]thiophene-2- carboxylate | 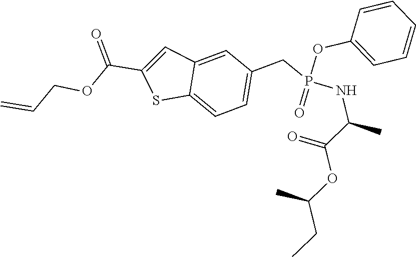 [SMILES: C=CCOC(=O)c1cc2cc(CP(=O)(N[C@@H](C)C(=O)O[C@H](C)CC)Oc3ccccc3)ccc2s1] | | ¹H NMR (400 MHz, CDCl3) δ 8.04 (d, J = 6.4 Hz, 1H), 7.90 − 7.79 (m, 2H), 7.48 (m, 1H), 7.30 − 7.27 (m, 1H), 7.25 (d, J = 2.0 Hz, 1H), 7.17 − 7.09 (m, 3H), 6.09 − 5.99 (m, 1H), 5.47 − 5.40 (m, 1H), 5.33 − 5.29 (m, 1H), 4.85 (d, J = 5.6 Hz, 2H), 4.81 − 4.71 (m, 1H), 4.01 − 3.90 (m, 1H), 3.48 − 3.38 (m, 2H), 1.55 − 1.43 (m, 2H), 1.18 − 1.14 (m, |
| | | | 3H), 1.14 − 1.09 (m |
| | | | 3H), 0.83 (s, 3H) |
| allyl 5-(((((S)-1-oxo-1- propoxypropan-2- yl)amino)(2,2,2- trifluoroethoxy) phosphoryl) methyl)benzo[b] thiophene-2- carboxylate | 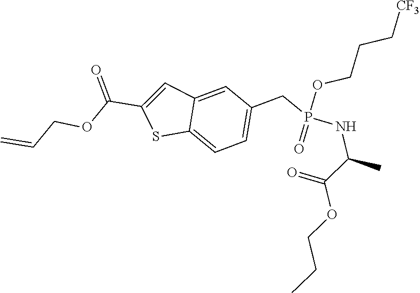 [SMILES: C=CCOC(=O)c1cc2cc(CP(=O)(N[C@@H](C)C(=O)OCCC)OCCCC(F)(F)F)ccc2s1] | | ¹H NMR (400 MHz, CDCl₃) δ 8.08 − 8.01 (m, 1H), 7.86 − 7.79 (m, 2H), 7.47 − 7.37 (m, 1H), 6.12 − 5.98 (m, 1H), 5.45 (dd, J = 1.6, 17.2 Hz, 1H), 5.33 (dd, J = 1.2, 10.4 Hz, 1H), 4.86 (d, J = 5.6 Hz, 2H), 4.33 − 4.16 (m, 2H), 4.10 − 3.93 (m, 2H), 3.43 − 3.31 (m, 2H), 3.21 − 2.96 (m, 1H), 1.66 − 1.54 (m, 3H), 1.35 (d, J = 7.2 Hz, 2H), 1.26 (d, J = 7.2 Hz, 1H), 0.95 − 0.88 (m, 3H) |
| allyl 5-(((((S)-1- ethoxy-1-oxo-3- phenylpropan-2- yl)amino)(phenoxy) phosphoryl)methyl) benzo[b]thiophene-2- carboxylate | 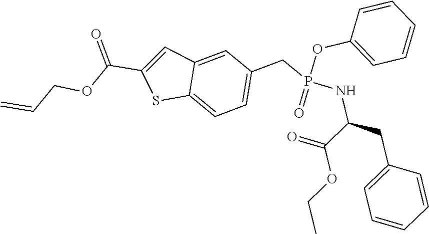 [SMILES: C=CCOC(=O)c1cc2cc(CP(=O)(N[C@@H](Cc3ccccc3)C(=O)OCC)Oc3ccccc3)ccc2s1] | 564 [M + H]⁺ | |
| allyl 5-(((((S)-1- (benzyloxy)-4-methyl- 1-oxopentan-2- yl)amino)(phenoxy) phosphoryl)methyl) benzo[b]thiophene-2- carboxylate | 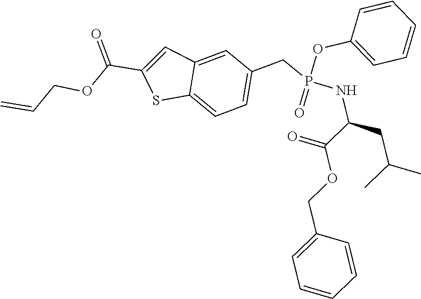 [SMILES: C=CCOC(=O)c1cc2cc(CP(=O)(N[C@@H](CC(C)C)C(=O)OCc3ccccc3)Oc3ccccc3)ccc2s1] | 592 [M + H]⁺ | |
| allyl 5-(((((S)-1- (benzyloxy)-1- oxopropan-2- yl)amino)(phenoxy) phosphoryl)methyl) benzo[b]thiophene-2- carboxylate | 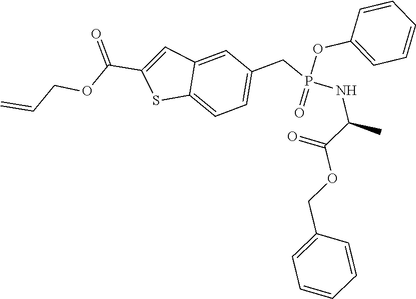 [SMILES: C=CCOC(=O)c1cc2cc(CP(=O)(N[C@@H](C)C(=O)OCc3ccccc3)Oc3ccccc3)ccc2s1] | 550 [M + H]⁺ | |
| isopropyl (((2- ((allyloxy)carbonyl) benzo[b]thiophen-5- yl)methyl)(phenoxy) phosphoryl)-L- prolinate | 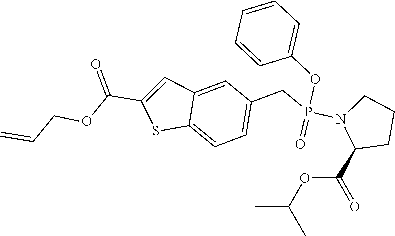 [SMILES: C=CCOC(=O)c1cc2cc(CP(=O)(Oc3ccccc3)N3CCC[C@H]3C(=O)OC(C)C)ccc2s1] | 528 [M + H]⁺ | |
| allyl 5-(((((S)-2- oxoTHF-3- yl)amino)(phenoxy) phosphoryl)methyl) benzo [b]thiophene-2- carboxylate | 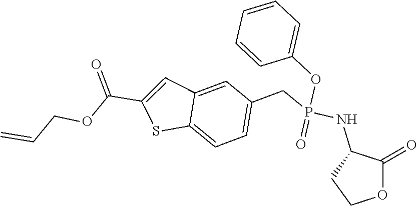 [SMILES: C=CCOC(=O)c1cc2cc(CP(=O)(N[C@H]3CCOC3=O)Oc3ccccc3)ccc2s1] | 472.1 [M + H]⁺ | |
| allyl 5-(((((S)-1- (benzyloxy)-1-oxo-4- phenylbutan-2- yl)amino)(phenoxy) phosphoryl)methyl) benzo[b]thiophene-2- carboxylate | 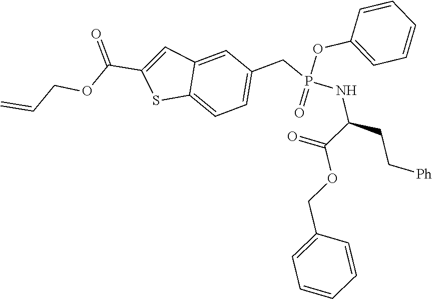 [SMILES: C=CCOC(=O)c1cc2cc(CP(=O)(N[C@@H](CCc3ccccc3)C(=O)OCc3ccccc3)Oc3ccccc3)ccc2s1] | 640 [M + H]⁺ | |
| allyl 5-(((((S)-1- ethoxy-1-oxopropan- 2- yl)amino)(phenoxy) phosphoryl)methyl) benzo[b]thiophene-2- carboxylate | 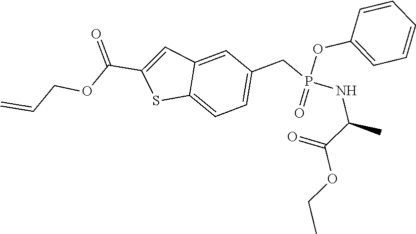 [SMILES: C=CCOC(=O)c1cc2cc(CP(=O)(N[C@@H](C)C(=O)OCC)Oc3ccccc3)ccc2s1] | 488 [M + H]⁺ | |
| allyl 5-(((((S)-3- methoxy-1-oxo-1- propoxypropan-2- yl)amino)(phenoxy) phosphoryl)methyl) benzo[b]thiophene-2- carboxylate | 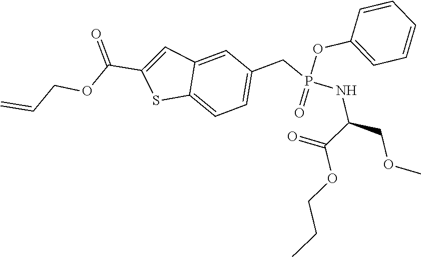 [SMILES: C=CCOC(=O)c1cc2cc(CP(=O)(N[C@@H](COC)C(=O)OCCC)Oc3ccccc3)ccc2s1] | 532 [M + H]⁺ | |
| allyl 5-(((((S)-4- methoxy-1-oxo-1- propoxybutan-2- yl)amino)(phenoxy) phosphoryl)methyl) benzo[b]thiophene-2- carboxylate | 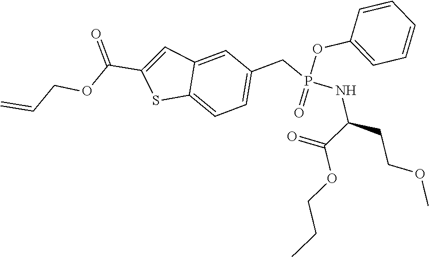 [SMILES: C=CCOC(=O)c1cc2cc(CP(=O)(N[C@@H](CCOC)C(=O)OCCC)Oc3ccccc3)ccc2s1] | 546 [M + H]⁺ | |
| allyl 5-(((((S)-1-oxo-1- propoxy-3-(pyridin-2- yl)propan-2- yl)amino)(phenoxy) phosphoryl)methyl) benzo[b]thiophene-2- carboxylate | 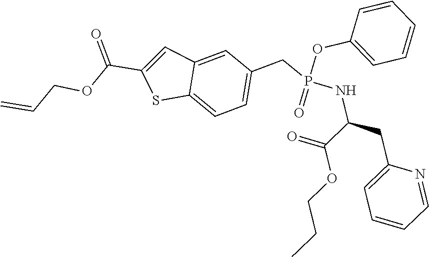 [SMILES: C=CCOC(=O)c1cc2cc(CP(=O)(N[C@@H](Cc3ccccn3)C(=O)OCCC)Oc3ccccc3)ccc2s1] | 579 [M + H]⁺ | |
| allyl 5-((phenoxy((1- (propoxycarbonyl) cyclopropyl)amino) phosphoryl) methyl)benzo[b] thiophene-2- carboxylate | 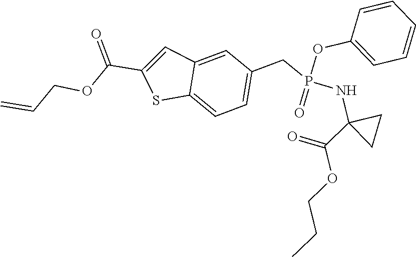 [SMILES: C=CCOC(=O)c1cc2cc(CP(=O)(NC3(C(=O)OCCC)CC3)Oc3ccccc3)ccc2s1] | 514.1 [M + H]⁺ | |

### Step 2: Preparation of 5-(((2,6-dimethylphenoxy)(((S)-1-oxo-1-propoxypropan-2-yl)amino)phosphoryl)methyl)benzo[b]thiophene-2-carboxylic acid

To a stirred solution of allyl 5-(((2,6-dimethylphenoxy)(((S)-1-oxo-1-propoxypropan-2-yl)amino)phosphoryl)methyl)benzo[b]thiophene-2-carboxylate (50 mg, 94.4 μmol, 1 eq) in THF (4 mL) were added morpholine (40.6 μL, 472 μmol, 5 eq) and Pd(PPh₃)₄(3.27 mg, 2.83 μmol, 0.03 eq) under nitrogen. The reaction mixture was stirred at room temperature 1 h. The solvent was removed under reduced pressure and the product was directly purified by reverse phase chromatography on a 100 g C₁₈ cartridge eluting with a gradient of 5-80% acetonitrile in water (with 0.1% formic acid) to afford 5-(((2,6-dimethylphenoxy)(((S)-1-oxo-1-propoxypropan-2-yl)amino)phosphoryl)methyl)benzo[b]thiophene-2-carboxylic acid (36.0 mg, 77.9%) as a pale yellow solid. ¹H NMR (400 MHz, CDCl₃) δ 7.93-7.84 (m, 1H), 7.83-7.71 (m, 2H), 7.48-7.36 (m, 1H), 7.08-6.93 (m, 3H), 4.17-3.98 (m, 3H), 3.59-3.42 (m, 2H), 2.37 (s, 3H), 2.30 (s, 3H), 1.65-1.57 (m, 2H), 1.27-1.22 (m, 3H), 1.15-1.11 (m, 1H), 0.94-0.88 (in, 3H).

The following intermediates in Table 59 were prepared using the method described above in step 2 for the preparation of 5-(((2,6-dimethylphenoxy)(((S)-1-oxo-1-propoxypropan-2-yl)amino)phosphoryl)methyl)benzo[b]thiophene-2-carboxylic acid and utilizing the appropriate starting materials and modifications.

TABLE 59

| Name | Structure | LCMS | NMR |
|---|---|---|---|
| 5-((((2-methyl-3-oxo- 3- propoxypropyl)amino) (phenoxy)phosphoryl) methyl)benzo[b]thioph ene-2-carboxylic acid | 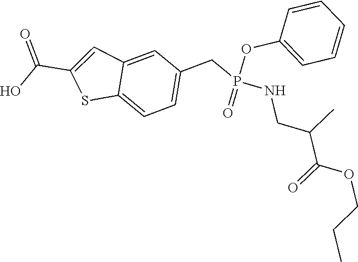 [SMILES: CCCOC(=O)C(C)CNP(=O)(Cc1ccc2sc(C(=O)O)cc2c1)Oc1ccccc1] | 476.2 [M + H]⁺ | ¹H NMR (400 MHz, DMSO-d₆) δ 13.39 (br. s., 1H), 8.10 − 8.05 (m, 1H), 8.01 − 7.88 (m, 2H), 7.51 − 7.44 (m, 1H), 7.39 − 7.28 (m, 2H), 7.22 − 7.05 (m, 3H), 5.22 − 5.10 (m, 1H), 3.96 − 3.82 (m, 2H), 3.47 − 3.36 (m, 2H), 3.10 − 2.94 (m, 1H), 2.86 − 2.70 (m, 1H), 2.36 − 2.22 (m, 1H), 1.56 − 1.45 (m, 2H), 0.90 (dd, J = 10.3, 7.1 Hz, 3H), 0.86 − 0.78 (m, 3H) |
| 5-((((S)-2- (butoxycarbonyl)- 4,4- difluoropyrrolidin-1- yl)(phenoxy) phosphoryl) methyl)benzo[b] thiophene-2- carboxylic acid | 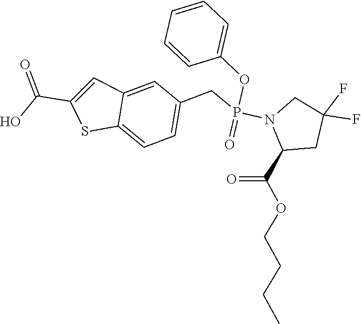 [SMILES: CCCCOC(=O)[C@@H]1CC(F)(F)CN1P(=O)(Cc1ccc2sc(C(=O)O)cc2c1)Oc1ccccc1] | 538.2 [M + H]⁺ | ¹H NMR (400 MHz, DMSO-d₆) δ 13.49 (br. s., 1H), 8.14 − 8.06 (m, 1H), 8.04 − 7.91 (m, 2H), 7.55 − 7.47 (m, 1H), 7.40 − 7.28 (m, 2H), 7.25 − 7.06 (m, 3H), 4.67 − 4.56 (m, 0.4H), 4.35 − 4.28 (m, 0.6H), 4.08 − 3.69 (m, 3H), 3.68 − 3.42 (m, 3H), 2.73 − 2.30 (m, 2H), 1.55 − 1.37 (m, 2H), 1.35 − 1.18 (m, 2H), 0.91 − 0.77 (m, 3H) |
| 5-(((((S)-1-oxo-1- propoxybutan-2- yl)amino)(phenoxy) phosphoryl)methyl) benzo[b]thiophene-2- carboxylic acid | 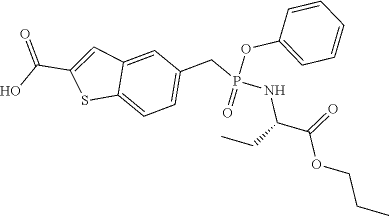 [SMILES: CCCOC(=O)[C@H](CC)NP(=O)(Cc1ccc2sc(C(=O)O)cc2c1)Oc1ccccc1] | 476.2 [M + H]⁺ | ¹H NMR (400 MHz, CD₃OD) δ 8.01 (s, 1H), 7.97 − 7.83 (m, 2H), 7.54 − 7.47 (m, 1H), 7.36 − 7.25 (m, 2H), 7.21 − 7.11 (m, 3H), 3.95 − 3.65 (m, 3H), 3.61 − 3.43 (m, 2H), 1.59 − 1.40 (m, 4H), 0.86 (q, J = 7.6 Hz, 3H), 0.73 − 0.63 (m, 3H) |
| 5-((((4-oxo-4- propoxybutan-2- yl)amino)(phenoxy) phosphoryl)methyl) benzo[b]thiophene-2- carboxylic acid | 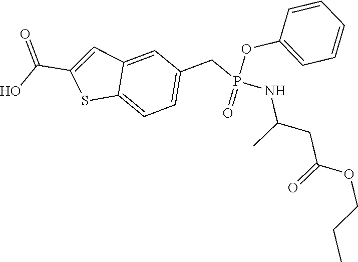 [SMILES: CCCOC(=O)CC(C)NP(=O)(Cc1ccc2sc(C(=O)O)cc2c1)Oc1ccccc1] | 476.2 [M + H]⁺ | ¹H NMR (400 MHz, DMSO-d₆) δ 13.47 (br. s., 1H), 8.08 (s, 1H), 8.02 − 7.89 (m, 2H), 7.53 − 7.45 (m, 1H), 7.39 − 7.27 (m, 2H), 7.17 − 7.09 (m, 3H), 5.22 − 5.04 (m, 1H), 3.92 − 3.80 (m, 2H), 3.61 − 3.45 (m, 1H), 3.45 − 3.25 (m, 3H), 2.30 − 2.13 (m, 2H), 1.55 − 1.44 (m, 2H), 0.93 − 0.86 (m, 3H), 0.85 − 0.79 (m, 3H) |
| 5-((((S)-2- (butoxycarbonyl) pyrrolidin-1- yl)(phenoxy) phosphoryl) methyl)benzo[b] thiophene- 2-carboxylic acid | 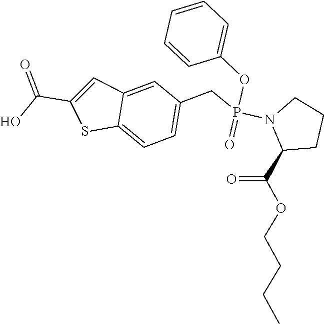 [SMILES: CCCCOC(=O)[C@@H]1CCCN1P(=O)(Cc1ccc2sc(C(=O)O)cc2c1)Oc1ccccc1] | 502.1 [M + H]⁺ | |
| 5-(((((S)-1-butoxy-1- oxopropan-2- yl)amino)(naphthalen- 1- yloxy)phosphoryl) methyl)benzo[b] thiophene-2- carboxylic acid | 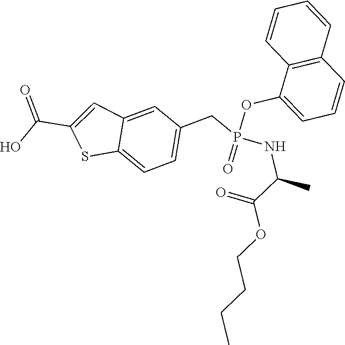 [SMILES: CCCCOC(=O)[C@H](C)NP(=O)(Cc1ccc2sc(C(=O)O)cc2c1)Oc1cccc2ccccc12] | 526.1 [M + H]⁺ | |
| 5-(((((S)-3-methyl-1- oxo-1-propoxybutan- 2- yl)amino)(phenoxy) phosphoryl) methyl)benzo [b]thiophene-2- carboxylic acid | 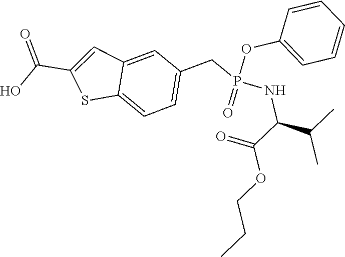 [SMILES: CCCOC(=O)[C@@H](NP(=O)(Cc1ccc2sc(C(=O)O)cc2c1)Oc1ccccc1)C(C)C] | 490.3 [M + H]⁺ | |
| 5-(((((S)-1-((R)-sec- butoxy)-1-oxopropan- 2- yl)amino)(phenoxy) phosphoryl)methyl) benzo[b]thiophene-2- carboxylic acid | 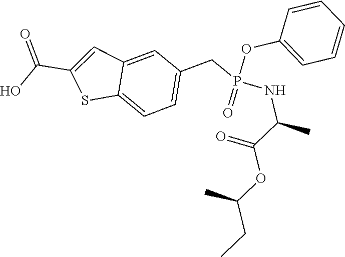 [SMILES: CC[C@@H](C)OC(=O)[C@H](C)NP(=O)(Cc1ccc2sc(C(=O)O)cc2c1)Oc1ccccc1] | 476.8 [M + H]⁺ | |
| 5-(((((S)-1-oxo-1- propoxypropan-2- yl)amino)(2,2,2- trifluoroethoxy) phosphoryl) methyl)benzo[b] thiophene-2- carboxylic acid | 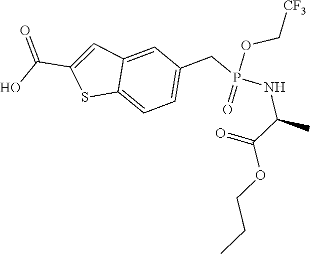 [SMILES: CCCOC(=O)[C@H](C)NP(=O)(Cc1ccc2sc(C(=O)O)cc2c1)OCC(F)(F)F] | | ¹H NMR (400 MHz, CDCl₃) δ 7.83 (s, 1H), 7.81 − 7.75 (m, 2H), 7.32 (dd, J = 2.0, 8.4 Hz, 1H), 4.50 − 4.29 (m, 2H), 4.18 − 3.98 (m, 2H), 3.87 − 3.62 (m, 1H), 3.46 − 3.32 (m, 2H), 1.72 − 1.64 (m, 2H), 1.42 (dd, J = 7.2, 17.2 Hz, 3H), 0.96 (q, J = 7.2 Hz, 3H) |
| 5-(((((S)-1-ethoxy-1- oxo-3-phenylpropan- 2- yl)amino)(phenoxy) phosphoryl)methyl) benzo[b]thiophene-2- carboxylic acid | 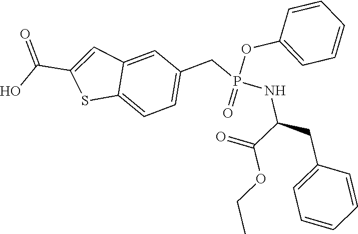 [SMILES: CCOC(=O)[C@H](Cc1ccccc1)NP(=O)(Cc1ccc2sc(C(=O)O)cc2c1)Oc1ccccc1] | 524 [M + H]⁺ | |
| 5-(((((S)-1- (benzyloxy)-4-methyl- 1-oxopentan-2- yl)amino)(phenoxy) phosphoryl)methyl) benzo[b]thiophene-2- carboxylic acid | 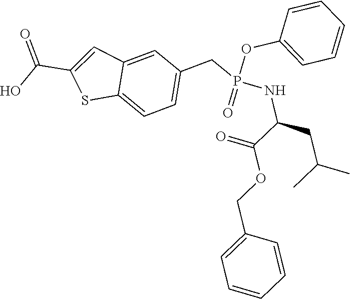 [SMILES: CC(C)C[C@H](NP(=O)(Cc1ccc2sc(C(=O)O)cc2c1)Oc1ccccc1)C(=O)OCc1ccccc1] | 552 [M + H]⁺ | |
| 5-(((((S)-1- (benzyloxy)-1- oxopropan-2- yl)amino)(phenoxy) phosphoryl)methyl) benzo[b]thiophene-2- carboxylic acid | 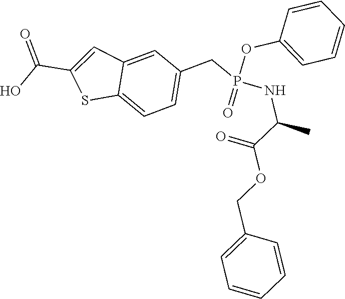 [SMILES: C[C@H](NP(=O)(Cc1ccc2sc(C(=O)O)cc2c1)Oc1ccccc1)C(=O)OCc1ccccc1] | 510 [M + H]⁺ | |
| 5-((((S)-2- (isopropoxycarbonyl) pyrrolidin-1- yl)(phenoxy) phosphoryl) methyl)benzo[b] thiophene- 2-carboxylic acid | 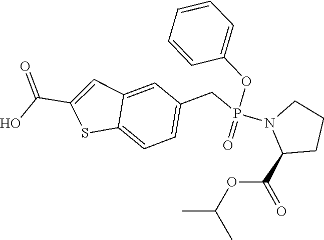 [SMILES: CC(C)OC(=O)[C@@H]1CCCN1P(=O)(Cc1ccc2sc(C(=O)O)cc2c1)Oc1ccccc1] | 488 [M + H]⁺ | |
| 5-(((((S)-2-oxoTHF-3- yl)amino)(phenoxy) phosphoryl)methyl) benzo[b]thiophene-2- carboxylic acid | 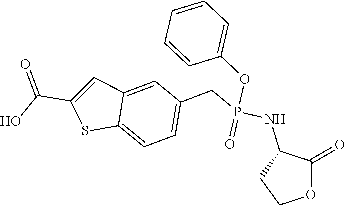 [SMILES: O=C(O)c1cc2cc(CP(=O)(N[C@H]3CCOC3=O)Oc3ccccc3)ccc2s1] | 432 [M + H]⁺ | |
| 5-(((((S)-1- (benzyloxy)-1-oxo-4- phenylbutan-2- yl)amino)(phenoxy) phosphoryl)methyl) benzo[b]thiophene-2- carboxylic acid | 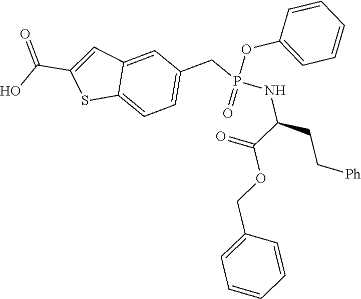 [SMILES: O=C(O)c1cc2cc(CP(=O)(N[C@@H](CCc3ccccc3)C(=O)OCc3ccccc3)Oc3ccccc3)ccc2s1] | 600 [M + H]⁺ | |
| 5-(((((S)-1-ethoxy-1- oxopropan-2- yl)amino)(phenoxy) phosphoryl)methyl) benzo[b]thiophene-2- carboxylic acid | 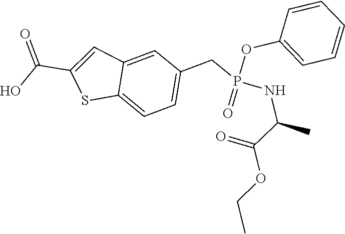 [SMILES: CCOC(=O)[C@H](C)NP(=O)(Cc1ccc2sc(C(=O)O)cc2c1)Oc1ccccc1] | 448 [M + H]⁺ | |
| 5-(((((S)-3-methoxy-1- oxo-1-propoxypropan- 2- yl)amino)(phenoxy) phosphoryl)methyl) benzo[b] thiophene-2- carboxylic acid | 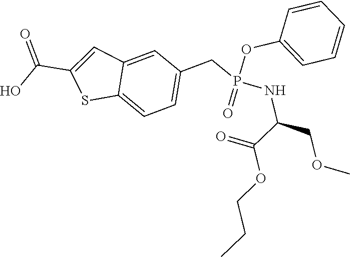 [SMILES: CCCOC(=O)[C@H](COC)NP(=O)(Cc1ccc2sc(C(=O)O)cc2c1)Oc1ccccc1] | 492 [M + H]⁺ | |
| 5-(((((S)-4-methoxy-1- oxo-1-propoxybutan- 2- yl)amino)(phenoxy) phosphoryl)methyl) benzo[b]thiophene-2- carboxylic acid | 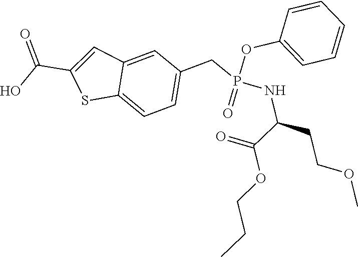 | 546 [M + H]⁺ | |
| 5-(((((S)-1-oxo-1- propoxy-3-(pyridin-2- yl)propan-2- yl)amino)(phenoxy) phosphoryl)methyl) benzo[b]thiophene-2- carboxylic acid | 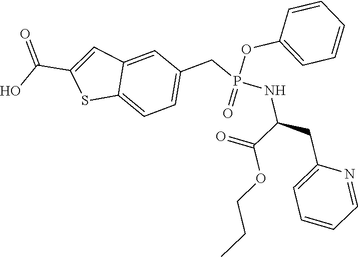 [SMILES: CCCOC(=O)[C@H](Cc1ccccn1)NP(=O)(Cc1ccc2sc(C(=O)O)cc2c1)Oc1ccccc1] | 539 [M + H]⁺ | |
| 5-((phenoxy((1- (propoxycarbonyl) cyclopropyl)amino) phosphoryl)methyl)benzo [b]thiophene-2-carboxylic acid | 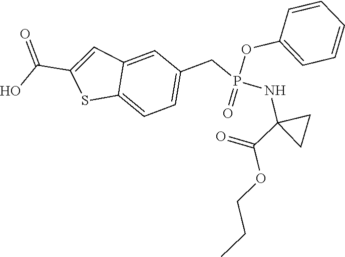 [SMILES: CCCOC(=O)C1(NP(=O)(Cc2ccc3sc(C(=O)O)cc3c2)Oc2ccccc2)CC1] | 474.0 [M + H]⁺ | |

### Synthesis of 5-((morpholino(((S)-1-oxo-1-propoxypropan-2-yl)amino)phosphoryl)methyl)benzo[b]thiophene-2-carboxylic acid

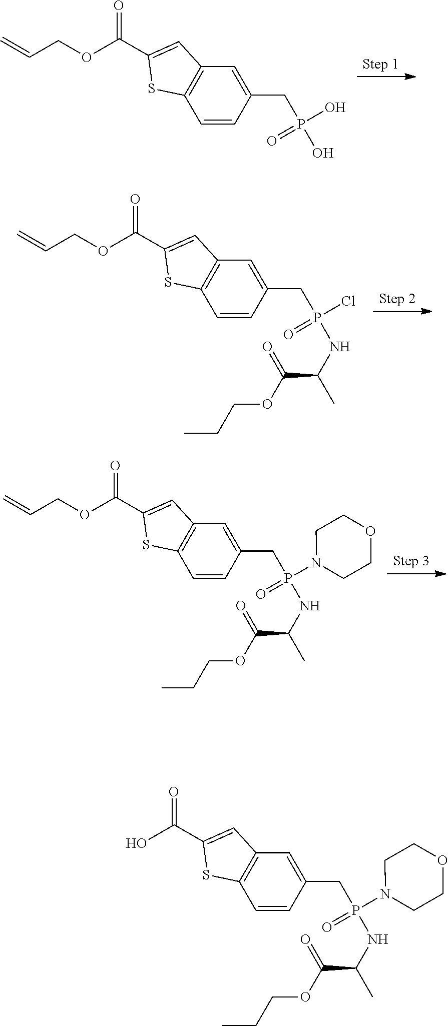 [SMILES: C=CCOC(=O)c1cc2cc(CP(=O)(O)O)ccc2s1 ; C=CCOC(=O)c1cc2cc(CP(=O)(Cl)N[C@@H](C)C(=O)OCCC)ccc2s1 ; C=CCOC(=O)c1cc2cc(CP(=O)(N[C@@H](C)C(=O)OCCC)N3CCOCC3)ccc2s1 ; CCCOC(=O)[C@H](C)NP(=O)(Cc1ccc2sc(C(=O)O)cc2c1)N1CCOCC1]

### Step 1: Synthesis of Allyl 5-((chloro(((S)-1-oxo-1-propoxypropan-2-yl)amino)phosphoryl)methyl)benzo[b]thiophene-2-carboxylate

Oxalyl chloride (10 mL) was added dropwise to the solution of ((2-((allyloxy)carbonyl)benzo[b]thiophen-5-yl)methyl)phosphonic acid (1 g, 3.20 mmol, 1.0 eq.) in dry DCM (20 mL) and DMF (1 drop) at 20° C. The reaction mixture was stirred at 25° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure to afford allyl 5-((dichlorophosphoryl)methyl)benzo[b]thiophene-2-carboxylate (960 mg, 2.74 mmol, 86%). allyl 5-((dichlorophosphoryl)methyl)benzo[b]thiophene-2-carboxylate (100 mg, 251 μmol, 1.0 eq.) was re-dissolved in anhydrous DCM (2 mL), then added to a mixture of propyl L-alaninate (36 mg, 276 μmol, 1.1 eq.) and triethylamine (101 mg, 1.00 mmol, 4.0 eq.) in anhydrous DCM (2 mL) at 0° C. The reaction was allowed to warm to 25° C., and stirred for an additional 15 min. The reaction progress was monitored by LCMS, and after completion, the reaction mixture was used in next step directly without further purification.

### Step 2: Synthesis of Allyl 5-((morpholino(((S)-1-oxo-1-propoxypropan-2-yl)amino)phosphoryl)methyl)benzo[b]thiophene-2-carboxylate

allyl 5-((chloro(((S)-1-oxo-1-propoxypropan-2-yl)amino)phosphoryl)methyl)benzo[b]thiophene-2-carboxylate (200 mg, 450 μmol, 1.0 eq.) as a solution in DCM (5 mL) was added to a solution of morpholine (196 mg, 2.25 mmol, 5 eq.) and TEA (273 mg, 2.7 mmol, 6 eq.) in DCM (1 mL). The mixture was stirred at 25° C. for 15 min, at which time LCMS showed formation of product. The mixture was concentrated under reduced pressure. The residue was purified by Biotage® C18 column chromatography to give allyl 5-((morpholino(((S)-1-oxo-1-propoxypropan-2-yl)amino)phosphoryl)methyl)benzo[b]thiophene-2-carboxylate (82 mg, 0.17 mmol, 38%) as a white solid. LCMS (ESI): m/z=495 [M+H]⁺.

The following intermediates in Table 60 were prepared using the method described above in step 2 for the preparation of allyl 5-((morpholino(((S)-1-oxo-1-propoxypropan-2-yl)amino)phosphoryl)methyl)benzo[b]thiophene-2-carboxylate and utilizing the appropriate starting materials and modifications.

TABLE 60

| Name | Structure | LCMS |
|---|---|---|
| allyl 5-(((2-methoxyethoxy)(((S)-1-oxo-1- propoxypropan-2- yl)amino)phosphoryl)methyl)benzo[b] thiophene-2-carboxylate | 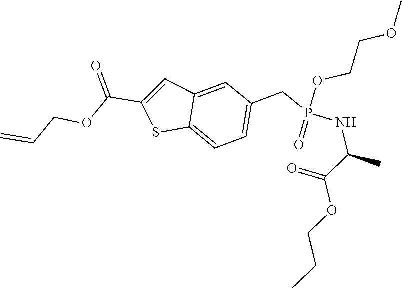 [SMILES: C=CCOC(=O)c1cc2cc(CP(=O)(N[C@@H](C)C(=O)OCCC)OCCOC)ccc2s1] | 484 [M + H]⁺ |
| allyl 5-((ethoxy(((S)-1-oxo-1-propoxypropan- 2- yl)amino)phosphoryl)methyl)benzo[b] thiophene-2-carboxylate | 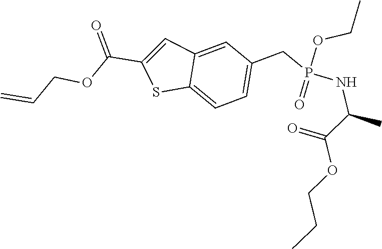 | 438 [M + H]⁺ |
| allyl 5-(((2,2-difluoropropoxy)(((S)-1-oxo-1- propoxypropan-2- yl)amino)phosphoryl)methyl)benzo[b] thiophene-2-carboxylate | 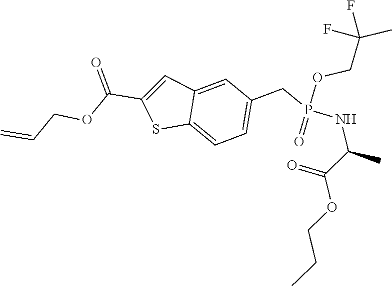 | 490 [M + H]⁺ |

### Step 3: Synthesis of 5-((morpholino(((S)-1-oxo-1-propoxypropan-2-yl)amino)phosphoryl)methyl)benzo[b]thiophene-2-carboxylic acid

To a solution of allyl 5-((morpholino(((S)-1-oxo-1-propoxypropan-2-yl)amino)phosphoryl)methyl)benzo[b]thiophene-2-carboxylate (82 mg, 0.17 mmol, 1.0 eq) in DCM (3 mL) were added Pd(PPh₃)₄(20 mg, 17 μmol, 0.1 eq.) and pyrrolidine (12 mg, 0.17 mmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 5-((morpholino(((S)-1-oxo-1-propoxypropan-2-yl)amino)phosphoryl)methyl)benzo[b]thiophene-2-carboxylic acid (50 mg, 0.11 mmol, 65%) as a white solid. LCMS (ESI): m/z=455 [M+H]⁺.

The following intermediates in Table 61 were prepared using the method described above in step 3 for the preparation of 5-((morpholino(((S)-1-oxo-1-propoxypropan-2-yl)amino)phosphoryl)methyl)benzo[b]thiophene-2-carboxylic acid and utilizing the appropriate starting materials and modifications.

TABLE 61

| Name | Structure | LCMS |
|---|---|---|
| 5-(((2-methoxyethoxy)(((S)-1-oxo-1- propoxypropan-2- yl)amino)phosphoryl)methyl)benzo[b] thiophene-2-carboxylic acid | 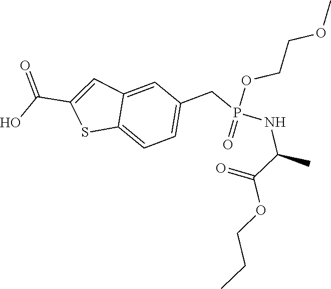 [SMILES: CCCOC(=O)[C@H](C)NP(=O)(Cc1ccc2sc(C(=O)O)cc2c1)OCCOC] | 444 [M + H]⁺ |
| 5-((ethoxy(((S)-1-oxo-1-propoxypropan-2- yl)amino)phosphoryl)methyl)benzo[b] thiophene-2-carboxylic acid | 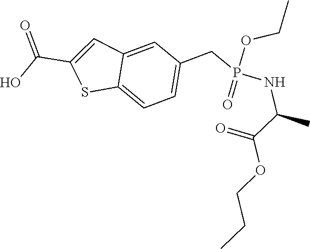 [SMILES: CCCOC(=O)[C@H](C)NP(=O)(Cc1ccc2sc(C(=O)O)cc2c1)OCC] | 414 [M + H]⁺ |
| 5-(((2,2-difluoropropoxy)(((S)-1-oxo-1- propoxypropan-2- yl)amino)phosphoryl)methyl)benzo[b] thiophene-2-carboxylic acid | 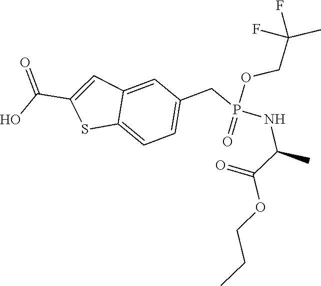 | 450 [M + H]⁺ |

### Synthesis of 5-(((((S)-4-methoxy-1-oxo-1-propoxybutan-2-yl)amino)(phenoxy)phosphoryl)methy)benzo[b]thiophene-2-carboxylic acid

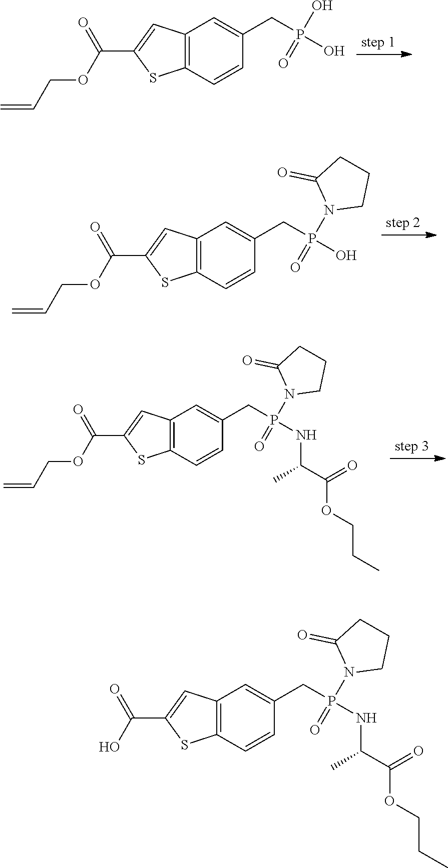 [SMILES: C=CCOC(=O)c1cc2cc(CP(=O)(O)O)ccc2s1 ; C=CCOC(=O)c1cc2cc(CP(=O)(O)N3CCCC3=O)ccc2s1 ; C=CCOC(=O)c1cc2cc(CP(=O)(N[C@@H](C)C(=O)OCCC)N3CCCC3=O)ccc2s1 ; CCCOC(=O)[C@H](C)NP(=O)(Cc1ccc2sc(C(=O)O)cc2c1)N1CCCC1=O]

### Step 1: Preparation of ((2-((allyloxy)carbonyl)benzo[b]thiophen-5-yl)methyl)(2-oxopyrrolidin-1-yl)phosphinic acid

Oxalyl chloride (10 mL) was added dropwise to the solution of ((2-((allyloxy)carbonyl)benzo[b]thiophen-5-yl)methyl)phosphonic acid (300 mg, 960 μmol, 1 eq.) in dry DCM (20 mL) and DMF (0.1 mL) at 0° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure to afford allyl 5-((dichlorophosphoryl)methyl)benzo[b]thiophene-2-carboxylate (300 mg). The residue (300 mg, 859 μmol, 1 eq.) was re-dissolved in anhydrous DCM (5 mL), then added to a mixture of pyrrolidin-2-one (218 mg, 2.57 mmol, 3 eq.) and TEA (259 mg, 2.57 mmol, 3 eq.) in anhydrous DCM (10 mL) at 0° C. The reaction was allowed to warm to 25° C. and stirred for an additional 2 h. After completion, the reaction mixture was concentrated under reduced pressure. The residue was purified by Biotage® C18 column chromatography to give ((2-((allyloxy)carbonyl)benzo[b]thiophen-5-yl)methyl)(2-oxopyrrolidin-1-yl)phosphinic acid (100 mg, 263 μmol, 31%) as a colorless oil. LCMS (ESI): m/z=380 [M+H]⁺.

### Step 2: Preparation of Allyl 5-(((((S)-1-oxo-1-propoxypropan-2-yl)amino)(2-oxopyrrolidin-1-yl)phosphoryl)methyl)benzo[b]thiophene-2-carboxylate

Oxalyl chloride (201 mg, 1.59 mmol, 5 eq.) was added dropwise to the solution of ((2-((allyloxy)carbonyl)benzo[b]thiophen-5-yl)methyl)(2-oxopyrrolidin-1-yl)phosphinic acid (100 mg, 320 μmol, 1 eq.) in dry DCM (5 mL) and DMF (0.1 mL) at 0° C. The reaction mixture was stirred at 40° C. for an additional 1 hr. The reaction was monitored by pipetting out a small amount of crude sample and quenching it with MeOH to ensure mono-C₁ phosphoryl chloride had been formed completely (mono-methoxy phosphonate was observed by LCMS). After completion, the excess oxalyl chloride and solvent were removed under reduced pressure to afford allyl 5-((chloro(2-oxopyrrolidin-1-yl)phosphoryl)methyl)benzo[b]thiophene-2-carboxylate (100 mg). The residue (100 mg, 251 μmol, 1 eq.) was re-dissolved in anhydrous DCM (10 mL), then added to a mixture of propyl L-alaninate (65.8 mg, 502 μmol, 2 eq.) and TEA (101 mg, 1.00 mmol, 4 eq.) in anhydrous DCM (10 mL) at 0° C. The reaction was allowed to warm to 25° C. and stirred for an additional 2 h. After completion, the reaction mixture was concentrated under reduced pressure. The residue was purified by Biotage® C18 column chromatography to give allyl 5-(((((S)-1-oxo-1-propoxypropan-2-yl)amino)(2-oxopyrrolidin-1-yl)phosphoryl)methyl)benzo[b]thiophene-2-carboxylate (23.0 mg, 46.6 μmol, 19%) as a colorless oil. LCMS (ESI): m/z=493 [M+H]⁺.

### Step 3: Preparation of 5-(((((S)-1-oxo-1-propoxypropan-2-yl)amino)(2-oxopyrrolidin-1-yl)phosphoryl)methyl)benzo[b]thiophene-2-carboxylic acid

To a solution of allyl 5-(((((S)-1-oxo-1-propoxypropan-2-yl)amino)(2-oxopyrrolidin-1-yl)phosphoryl)methyl)benzo[b]thiophene-2-carboxylate (22 mg, 44.6 μmol, 1 eq) in DCM (2 mL) were added Pd(PPh₃)₄(5 mg, 4.46 μmol, 0.1 eq.) and pyrrolidine (3 mg, 44.6 μmol, 1.0 eq.). The mixture was purged and degassed with N₂ (three times), then stirred at 25° C. for 0.5 h. After completion, the mixture was quenched with HCl aqueous solution (1 N) and extracted with DCM (10 mL×3). The organic layers were combined and washed with brine (15 mL), dried over anhydrous Na₂SO₄, and concentrated under reduced pressure. The residue was purified by Biotage® C18 column chromatography to give 5-(((((S)-4-methoxy-1-oxo-1-propoxybutan-2-yl)amino)(phenoxy)phosphoryl)methyl)benzo[b]thiophene-2-carboxylic acid (13.0 mg, 28.7 μmol, 65%) as a colorless oil. LCMS (ESI): m/z=453 [M+H]⁺.

### Synthesis of 5-(1-((((S)-1-oxo-1-propoxypropan-2-yl)amino)(phenoxy)phosphoryl)ethyl)benzo[b]thiophene-2-carboxylic acid

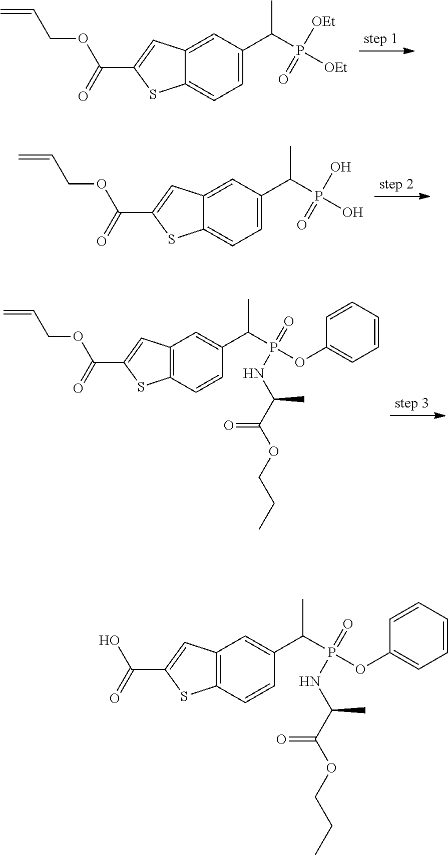 [SMILES: C=CCOC(=O)c1cc2cc(C(C)P(=O)(OCC)OCC)ccc2s1 ; C=CCOC(=O)c1cc2cc(C(C)P(=O)(O)O)ccc2s1 ; C=CCOC(=O)c1cc2cc(C(C)P(=O)(N[C@@H](C)C(=O)OCCC)Oc3ccccc3)ccc2s1 ; CCCOC(=O)[C@H](C)NP(=O)(Oc1ccccc1)C(C)c1ccc2sc(C(=O)O)cc2c1]

### Step 1: Preparation of Allyl 5-(1-(diethoxyphosphoryl)ethyl)benzo[b]thiophene-2-carboxylate

To a solution of (1-(2-((allyloxy)carbonyl)benzo[b]thiophen-5-yl)ethyl)phosphonic acid (400 mg, 1.05 mmol, 1.0 eq.) in DCM (10 mL) were added TMSBr (1.6 g, 10.5 mmol, 10.0 eq.). The mixture was stirred at room temperature for 16 h. After completion, the reaction mixture was concentrated under reduced pressure. The residue was purified by flash column chromatography on silica gel to afford allyl 5-(1-(diethoxyphosphoryl)ethyl)benzo[b]thiophene-2-carboxylate (310 mg, 0.95 mmol, 90%) as a colorless oil. LCMS (ESI): m/z=327 [M+H]⁺.

### Step 2: Preparation of Allyl 5-(1-((((S)-1-oxo-1-propoxypropan-2-yl)amino)(phenoxy)phosphoryl)ethyl)benzo[b]thiophene-2-carboxylate

Oxalyl chloride (599 mg, 4.75 mmol, 5 eq.) was added dropwise to the solution of allyl 5-(1-(diethoxyphosphoryl)ethyl)benzo[b]thiophene-2-carboxylate (310 mg, 0.95 mmol, 1 eq.) in dry DCM (20 mL) and DMF (0.1 mL) at 25° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (20 mL), then added to a mixture of phenol (89 mg, 0.95 mmol, 1.0 eq.) and TEA (960 mg, 9.5 mmol, 10 eq.) in anhydrous DCM (20 mL) at −20° C. The reaction was stirred at −20° C. for an additional 1 hr. Then, propyl L-alaninate (249 mg, 1.90 mmol, 2.0 eq.) was added into the above mixture at 0° C. The reaction was stirred at 25° C. for an additional 1 hr. After completion, the reaction was poured into ice-water (10 mL) and extracted with DCM (10 mL×2). The organic layers were combined and washed with brine (10 mL), dried over anhydrous Na₂SO₄, and concentrated under reduced pressure. The residue was purified by Biotage® C18 column chromatography to afford allyl 5-(1-((((S)-1-oxo-1-propoxypropan-2-yl)amino)(phenoxy)phosphoryl)ethyl)benzo[b]thiophene-2-carboxylate (110 mg, 0.21 mmol, 22%) as a brown solid. LCMS (ESI): m/z=516 [M+H]⁺.

### Step 3: Preparation of 5-(1-((((S)-1-oxo-1-propoxypropan-2-yl)amino)(phenoxy)phosphoryl)ethyl)benzo[b]thiophene-2-carboxylic acid

To a solution of allyl 5-(1-((((S)-1-oxo-1-propoxypropan-2-yl)amino)(phenoxy)phosphoryl)ethyl)benzo[b]thiophene-2-carboxylate (110 mg, 0.21 mmol, 1.0 eq) in DCM (10 mL) were added Pd(PPh₃)₄(24 mg, 0.02 mmol, 0.1 eq.) and pyrrolidine (15 mg, 0.21 mmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 5-(1-((((S)-1-oxo-1-propoxypropan-2-yl)amino)(phenoxy)phosphoryl)ethyl)benzo[b]thiophene-2-carboxylic acid (68 mg, 0.13 mmol, 62%) as a yellow solid. LCMS (ESI): m/z=516 [M+H]⁺.

### Synthesis of 7-(((((S)-1-(benzyloxy)-1-oxopropan-2-yl)amino)(phenoxy)phosphoryl)methyl)-2-naphthoic acid

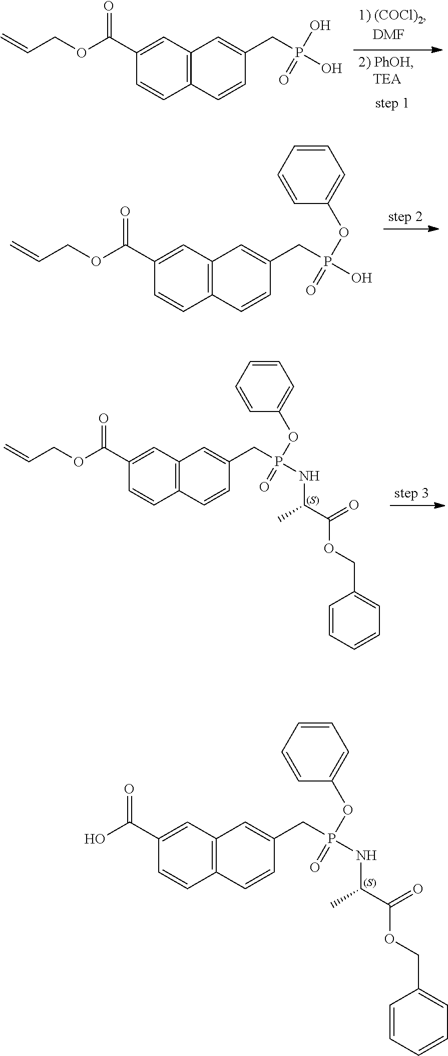

### Step 1: Preparation of Allyl 7-((hydroxy(phenoxy)phosphoryl)methyl)-2-naphthoate

Oxalyl chloride (208 mg, 1.65 mmol, 5 eq.) was added dropwise to the solution of ((7-((allyloxy)carbonyl)naphthalen-2-yl)methyl)phosphonic acid (100 mg, 0.33 mmol, 1 eq.) in dry DCM (20 mL) and DMF (0.1 mL) at 25° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (20 mL), then added to a mixture of phenol (31 mg, 0.33 mmol, 1.0 eq.) and TEA (333 mg, 3.3 mmol, 10 eq.) in anhydrous DCM (20 mL) at −20° C. The reaction was stirred at 25° C. for an additional 1 hr. After completion, the reaction was poured into ice-water (50 mL) and extracted with DCM (50 mL×2). The organic layers were combined and washed with brine (10 mL), dried over anhydrous Na₂SO₄, and concentrated under reduced pressure. The residue was purified by Biotage® C18 column chromatography to afford allyl 7-((hydroxy(phenoxy)phosphoryl)methyl)-2-naphthoate (100 mg, 0.26 mmol, 79%) as a yellow oil. LCMS (ESI): m/z=383 [M+H]⁺.

### Step 2: Preparation of Allyl 7-(((((S)-1-(benzyloxy)-1-oxopropan-2-yl)amino)(phenoxy)phosphoryl)methyl)-2-naphthoate

Oxalyl chloride (330 mg, 2.62 mmol, 5 eq.) was added dropwise to the solution of allyl 7-((hydroxy(phenoxy)phosphoryl)methyl)-2-naphthoate (200 mg, 0.52 mmol, 1 eq.) in dry DCM (10 mL) and DMF (0.1 mL) at 25° C. The reaction mixture was stirred at 40° C. for an additional 1 hr. After completion, the excess oxalyl chloride and solvent were removed under reduced pressure. The residue was re-dissolved in anhydrous DCM (10 mL), then added to a mixture of benzyl L-alaninate (186 mg, 1.04 mmol, 2.0 eq.) and triethylamine (525 mg, 5.2 mmol, 10 eq.) in anhydrous DCM (20 mL) at 0° C. The reaction was stirred at 25° C. for an additional 1 hr. After completion, the reaction was poured into ice-water (10 mL) and extracted with DCM (10 mL×2). The organic layers were combined and washed with brine (10 mL), dried over anhydrous Na₂SO₄, and concentrated under reduced pressure. The residue was purified by Biotage® C18 column chromatography to afford allyl 7-(((((S)-1-(benzyloxy)-1-oxopropan-2-yl)amino)(phenoxy)phosphoryl)methyl)-2-naphthoate (50 mg, 92 μmol, 18%) as a brown solid. LCMS (ESI): m/z=544 [M+H]⁺.

The following intermediates in Table 62 were prepared using the method described above in step 2 for the preparation of allyl 7-(((((S)-1-(benzyloxy)-1-oxopropan-2-yl)amino)(phenoxy)phosphoryl)methyl)-2-naphthoate and utilizing the appropriate starting materials and modifications.

TABLE 62

| Name | Structure | LCMS |
|---|---|---|
| allyl 7-(((((S)-1-ethoxy-1-oxopropan-2- yl)amino)(phenoxy)phosphoryl)methyl)-2- naphthoate | 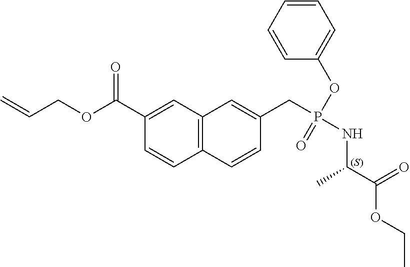 | 482 [M + H]⁺ |
| allyl 7-(((((S)-1-oxo-1-propoxypropan-2- yl)amino)(phenoxy)phosphoryl)methyl)-2- naphthoate | 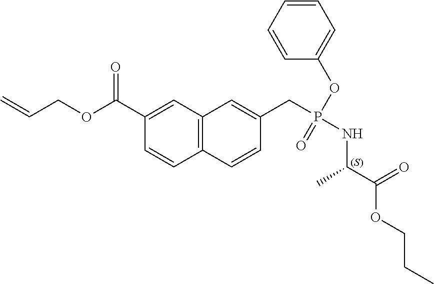 | 496 [M + H]⁺ |
| allyl 7-(((((S)-1-(2-ethylbutoxy)-1-oxopropan- 2-yl)amino)(phenoxy)phosphoryl)methyl)-2- naphthoate | 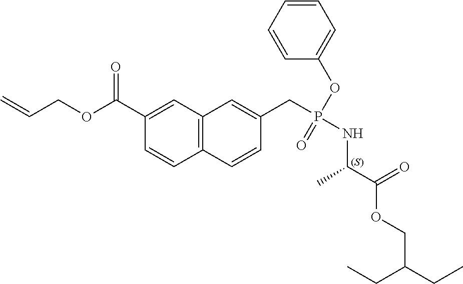 | 538.3 [M + H]⁺ |
| allyl 7-(((((S)-1-butoxy-1-oxopropan-2- yl)amino)(phenoxy)phosphoryl)methyl)-2- naphthoate | 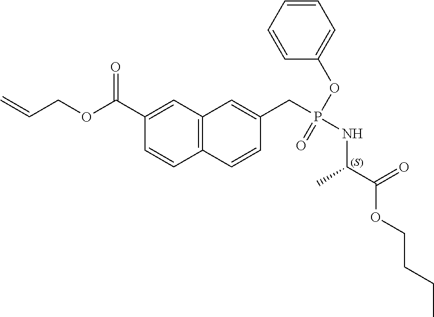 | 510 [M + H]⁺ |
| allyl 7-(((((S)-1-isobutoxy-1-oxopropan-2- yl)amino)(phenoxy)phosphoryl)methyl)-2- naphthoate | 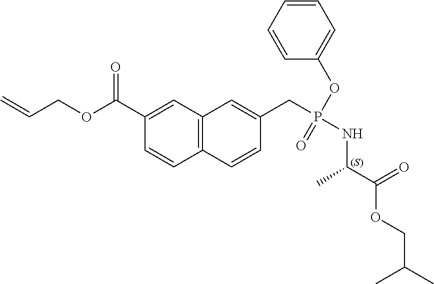 | 510.1 [M + H]⁺ |
| allyl 7-(((((S)-1-butoxy-1-oxopropan-2- yl)amino)(naphthalen-1- yloxy)phosphoryl)methyl)-2-naphthoate | 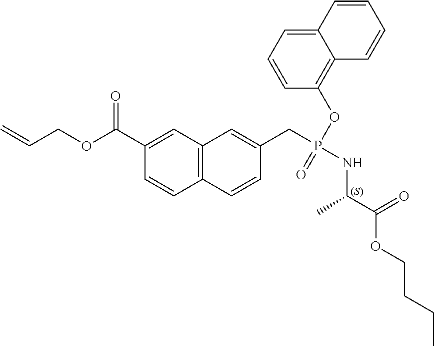 | 560 [M + H]⁺ |
| allyl 7-(((((S)-1-isopropoxy-1-oxopropan-2- yl)amino)(phenoxy)phosphoryl)methyl)-2- naphthoate | 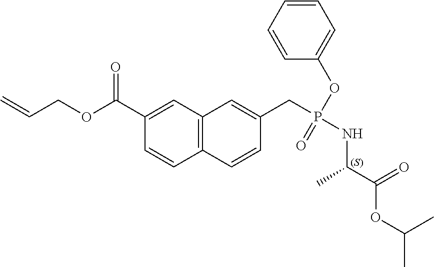 | 496 [M + H]⁺ |
| allyl 7-(((((S)-1-(neopentyloxy)-1-oxopropan- 2-yl)amino)(phenoxy)phosphoryl)methyl)-2- naphthoate | 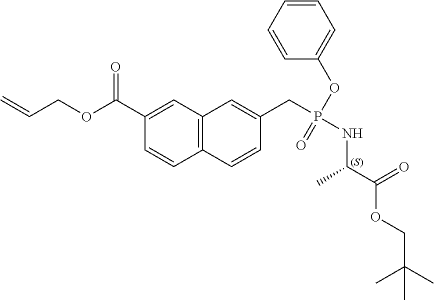 | 524.0 [M + H]⁺ |

### Step 3: Preparation of 7-(((((S)-1-(benzyloxy)-1-oxopropan-2-yl)amino)(phenoxy)phosphoryl)methyl)-2-naphthoic acid

To a solution of allyl 7-(((((S)-1-(benzyloxy)-1-oxopropan-2-yl)amino) (phenoxy)phosphoryl)methyl)-2-naphthoate (50 mg, 92 μmol, 1.0 eq) in DCM (10 mL) were added Pd(PPh₃)₄(11 mg, 9.2 μmol, 0.1 eq.) and pyrrolidine (7 mg, 92 μmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 7-(((((S)-1-(benzyloxy)-1-oxopropan-2-yl)amino) (phenoxy)phosphoryl)methyl)-2-naphthoic acid (40 mg, 80 μmol, 87%) as a yellow solid. LCMS (ESI): m/z=504 [M+H]⁺.

The following intermediates in Table 63 were prepared using the method described above in step 3 for the preparation of allyl 7-(((((S)-1-(benzyloxy)-1-oxopropan-2-yl)amino) (phenoxy)phosphoryl)methyl)-2-naphthoate and utilizing the appropriate starting materials and modifications.

TABLE 63

| Name | Structure | LCMS |
|---|---|---|
| 7-(((((S)-1-ethoxy-1-oxopropan-2- yl)amino)(phenoxy)phosphoryl)methyl)-2- naphthoic acid | 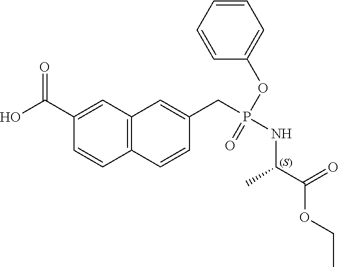 | 442 [M + H]⁺ |
| 7-(((((S)-1-oxo-1-propoxypropan-2- yl)amino)(phenoxy)phosphoryl)methyl)-2- naphthoic acid | 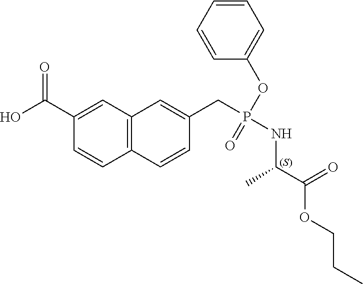 | 456 [M + H]⁺ |
| 7-(((((S)-1-(2-ethylbutoxy)-1-oxopropan-2- yl)amino)(phenoxy)phosphoryl)methyl)-2- naphthoic acid | 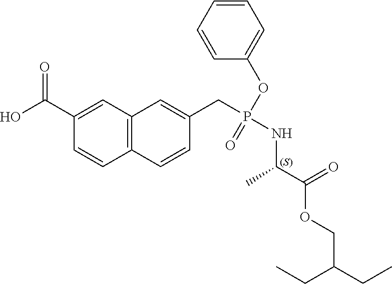 | 498.2 [M + H]⁺ |
| 7-(((((S)-1-butoxy-1-oxopropan-2- yl)amino)(phenoxy)phosphoryl)methyl)-2- naphthoic acid | 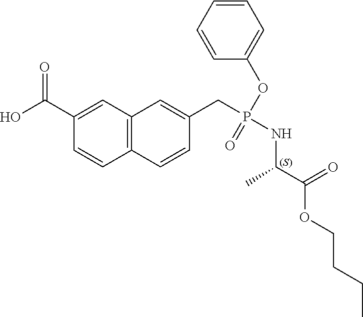 | 470 [M + H]⁺ |
| 7-(((((S)-1-isobutoxy-1-oxopropan-2- yl)amino)(phenoxy)phosphoryl)methyl)-2- naphthoic acid | 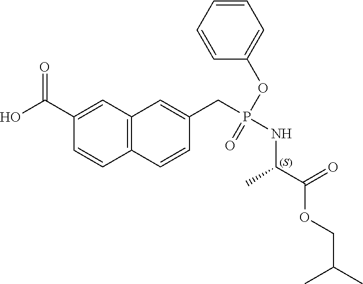 | 470.1 [M + H]⁺ |
| 7-(((((S)-1-butoxy-1-oxopropan-2- yl)amino)(naphthalen-1- yloxy)phosphoryl)methyl)-2-naphthoic acid | 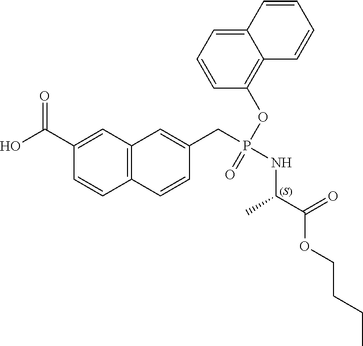 | 520 [M + H]⁺ |
| 7-(((((S)-1-isopropoxy-1-oxopropan-2- yl)amino)(phenoxy)phosphoryl)methyl)-2- naphthoic acid | 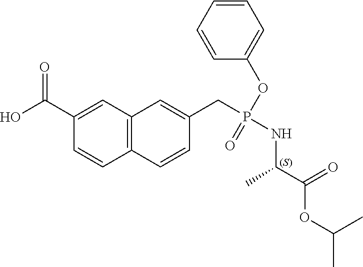 | 456 [M + H]⁺ |
| 7-(((((S)-1-(neopentyloxy)-1-oxopropan-2- yl)amino)(phenoxy)phosphoryl)methyl)-2- naphthoic acid | 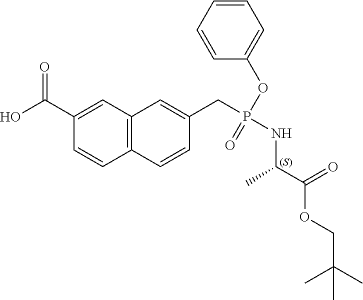 | 484.2 [M + H]⁺ |

### Synthesis of 7-(1-((((S)-1-oxo-1-propoxypropan-2-yl)amino)(phenoxy)phosphoryl)ethyl)-2-napthoic acid

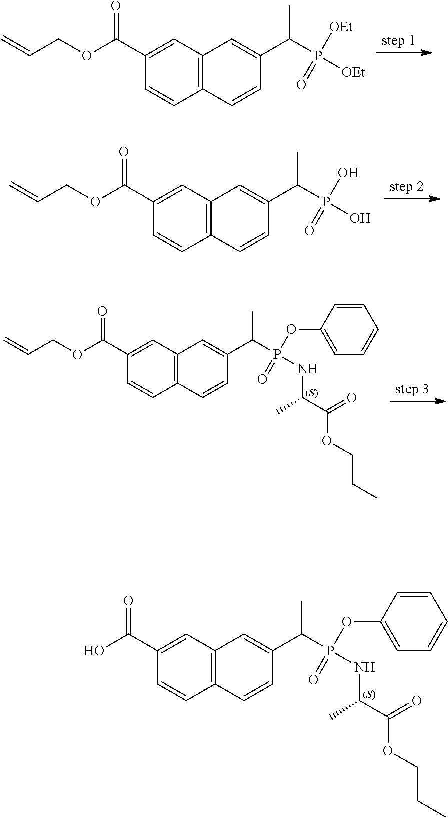

### Step 1: Preparation of (1-(7-((allyloxy)carbonyl)naphthalen-2-yl)ethyl)phosphonic acid

To a solution allyl 7-(1-(diethoxyphosphoryl)ethyl)-2-naphthoate (600 mg, 1.60 mmol, 1 eq.) in DCM (5 mL) was added TMSBr (2.5 g, 16.0 mmol, 10 eq.). The mixture was stirred at room temperature for 2 h. After completion, the reaction mixture was concentrated under reduced pressure. The residue was purified by flash column chromatography on silica gel to afford (1-(7-((allyloxy)carbonyl)naphthalen-2-yl)ethyl)phosphonic acid (400 mg, 1.25 mmol, 78%) as a white solid. LCMS (ESI): m/z=321 [M+H]⁺.

### Step 2: Preparation of Allyl 7-(1-((((S)-1-oxo-1-propoxypropan-2-yl)amino)(phenoxy)phosphoryl)ethyl)-2-naphthoate

Oxalyl chloride (616 mg, 4.89 mmol, 3 eq.) was added dropwise to the solution of a (1-(7-((allyloxy)carbonyl)naphthalen-2-yl)ethyl)phosphonic acid (520 mg, 1.63 mmol, 1 eq.) in dry DCM (5 mL) and DMF (0.1 mL) at 0° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (10 mL), then added to a mixture of phenol (460 mg, 4.89 mmol, 3 eq.) and TEA (1.4 g, 13.0 mmol, 8 eq.) in anhydrous DCM (10 mL) at 0° C. The reaction was allowed to warm to 25° C. and stirred for an additional 2 h. After completion, the reaction mixture was concentrated under reduced pressure. The residue was purified by Biotage® C18 column chromatography to give allyl 7-(1-((((S)-1-oxo-1-propoxypropan-2-yl)amino)(phenoxy)phosphoryl)ethyl)-2-naphthoate (120 mg, 0.24 mmol, 15%) as a colorless oil. LCMS (ESI): m/z=510 [M+H]⁺.

### Step 3: Preparation of 7-(1-((((S)-1-oxo-1-propoxypropan-2-yl)amino)(phenoxy)phosphoryl)ethyl)-2-naphthoic acid

To a solution of allyl 7-(1-((((S)-1-oxo-1-propoxypropan-2-yl)amino)(phenoxy)phosphoryl)ethyl)-2-naphthoate (120 mg, 0.24 mmol, 1.0 eq) in DCM (1 mL) were added Pd(PPh₃)₄(28 mg, 24 μmol, 0.1 eq.) and pyrrolidine (17 mg, 0.24 mmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 7-(1-((((S)-1-oxo-1-propoxypropan-2-yl)amino)(phenoxy)phosphoryl)ethyl)-2-naphthoic acid (82 mg, 0.17 mmol, 71%) as a white solid. LCMS (ESI): m/z=470 [M+H]⁺.

### Synthesis of 5-((bis((S)-2-(propoxycarbonyl)pyrrolidin-1-yl)phosphoryl)methyl)benzo[b]thiophene-2-carboxylic acid

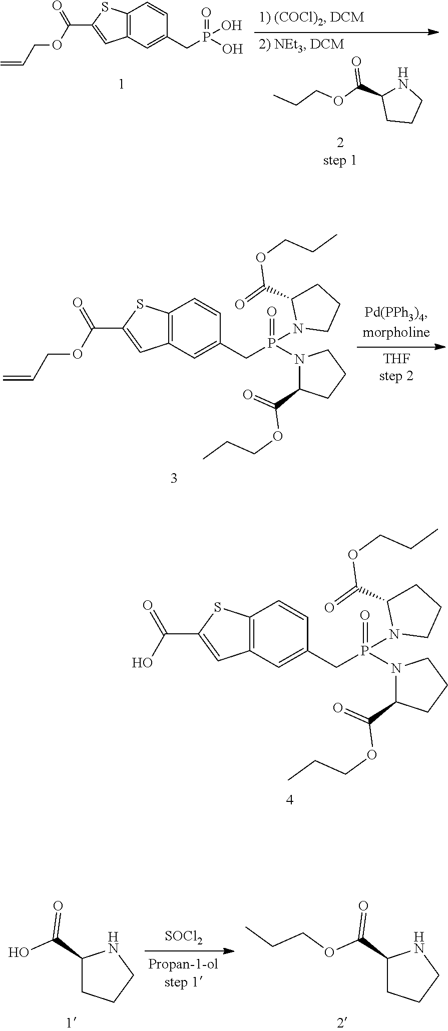

### Step 1: Preparation of Propyl L-Prolinate

To a solution of (2S)-pyrrolidine-2-carboxylic acid (2.00 g, 17.3 mmol, 1 eq) in propan-1-ol (13.0 mL, 266 mmol) at −78° C. was added dropwise thionyl chloride (3.76 mL, 51.9 mmol, 3 eq). The mixture was allowed to warm to room temperature and then heated at 80° C. overnight. The reaction mixture was concentrated under reduced pressure and the crude propyl L-prolinate (2.62 g, 16.6 mmol, 96.6%) was isolated as an orange sticky oil that was used directly in the next step. LCMS (ESI): m/z=158.1 [M+H]⁺.

### Step 2: Preparation of Dipropyl (((2-((allyloxy)carbonyl)benzo[b]thiophen-5-yl)methyl)phosphoryl)(S)-di-L-prolinate

To a solution of ((2-((allyloxy)carbonyl)benzo[b]thiophen-5-yl)methyl)phosphonic acid (300 mg, 0.9606 mmol, 1 eq) in DCM (10 mL) at 0° C. were added 2 drops of DMF (cat) and oxalyl chloride (246 μL, 2.88 mmol, 3 eq). The solution was stirred at 0° C. and was allowed to warm slowly to room temperature overnight. The reaction was concentrated under reduced pressure and then dried under high vacuum during 30 min. The crude residue was diluted in DCM (10 mL, dried with Na₂SO₄) and cooled down to 0° C. A solution of propyl L-prolinate (603 mg, 3.84 mmol, 4 eq) and triethylamine (668 μL, 4.80 mmol, 5 eq) in DCM (3 mL, dried with Na₂SO₄) was added and the mixture was stirred at room temperature for 3 d. The reaction was concentrated under reduced pressure and the crude residue was purified by reverse phase chromatography on a 150 g C₁₈ cartridge eluting with 5-100% MeCN in water (with 0.1% formic acid) to give propyl dipropyl (((2-((allyloxy)carbonyl)benzo[b]thiophen-5-yl)methyl)phosphoryl)(S)-di-L-prolinate (274 mg, 48.3%) as an orange oil. LCMS (ESI): m/z=591.2 [M+H]⁺.

The following intermediates in Table 64 were prepared using the method described above in step 2 for the preparation of dipropyl (((2-((allyloxy)carbonyl)benzo[b]thiophen-5-yl)methyl)phosphoryl)(S)-di-L-prolinate and utilizing the appropriate starting materials and modifications.

TABLE 64

| Name | Structure | LCMS |
|---|---|---|
| dibutyl 2,2′-((((2- ((allyloxy)carbonyl)benzo[b]thiophen- 5-yl)methyl)phosphoryl)bis(azanediyl)) (2S,2′S)-dipropionate | 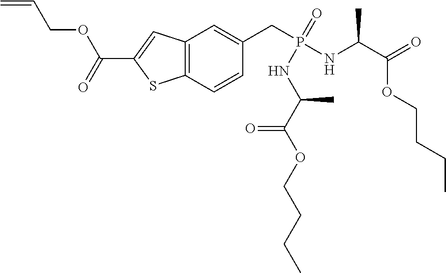 [SMILES: C=CCOC(=O)c1cc2cc(CP(=O)(N[C@@H](C)C(=O)OCCCC)N[C@@H](C)C(=O)OCCCC)ccc2s1] | 567 [M + H]⁺ |

### Step 3: Preparation of 5-((bis((S)-2-(propoxycarbonyl)pyrrolidin-1-yl)phosphoryl)methyl)benzo[b]thiophene-2-carboxylic acid

To a solution of propyl dipropyl (((2-((allyloxy)carbonyl)benzo[b]thiophen-5-yl)methyl)phosphoryl)(S)-di-L-prolinate (274 mg, 0.4638 mmol, 1 eq) in THF (30 mL) were added morpholine (199 μL, 2.31 mmol, 5 eq) and Pd(PPh₃)₄(53.5 mg, 0.04638 mmol, 0.10 eq). The reaction mixture was stirred at room temperature for 2 h and THF was removed under reduced pressure. The crude residue was purified by reverse phase chromatography on a 100 g C₁₈ cartridge eluting with 5-100% MeCN in water (with 0.1% formic acid) to give 5-((bis((S)-2-(propoxycarbonyl)pyrrolidin-1-yl)phosphoryl)methyl)benzo[b]thiophene-2-carboxylic acid (203 mg, 79.6%) as a white solid. LCMS (ESI): m/z=551.2 [M+H]⁺.

The following intermediates in Table 65 were prepared using the method described above in step 2 for the preparation of 5-((bis((S)-2-(propoxycarbonyl)pyrrolidin-1-yl)phosphoryl)methyl)benzo[b]thiophene-2-carboxylic acid and utilizing the appropriate starting materials and modifications.

TABLE 65

| Name | Structure | LCMS |
|---|---|---|
| 5-((bis(((S)-1-butoxy-1-oxopropan-2-yl) amino)phosphoryl)methyl)benzo[b] thiophene-2-carboxylic acid | 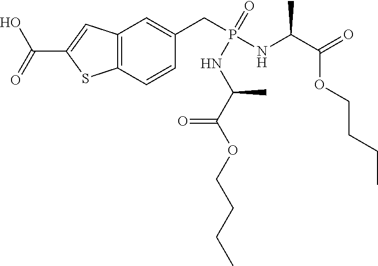 [SMILES: CCCCOC(=O)[C@H](C)NP(=O)(Cc1ccc2sc(C(=O)O)cc2c1)N[C@@H](C)C(=O)OCCCC] | 527 [M + H]⁺ |

### Synthesis of 5-(((((S)-1-isopropoxy-3-methoxy-1-oxopropan-2-yl)amino)(phenoxy)phosphoryl)methyl)benzo[b]thiophene-2-carboxylic acid

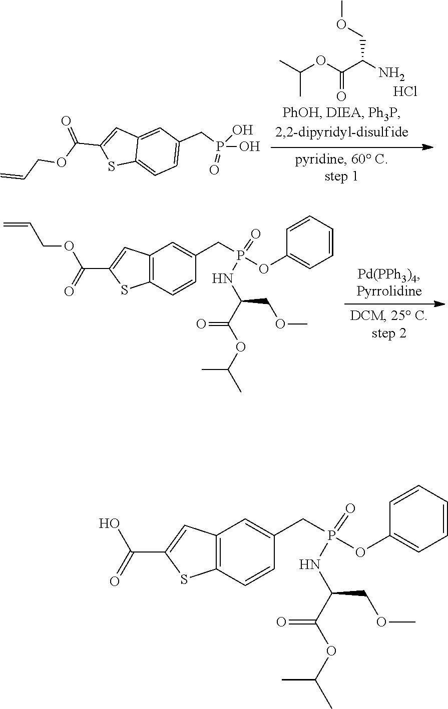 [SMILES: COC[C@H](N)C(=O)OC(C)C ; Cl ; C=CCOC(=O)c1cc2cc(CP(=O)(O)O)ccc2s1 ; C=CCOC(=O)c1cc2cc(CP(=O)(N[C@@H](COC)C(=O)OC(C)C)Oc3ccccc3)ccc2s1 ; COC[C@H](NP(=O)(Cc1ccc2sc(C(=O)O)cc2c1)Oc1ccccc1)C(=O)OC(C)C]

### Step 1: Preparation of Allyl 5-(((((S)-1-isopropoxy-3-methoxy-1-oxopropan-2-yl)amino)(phenoxy)phosphoryl)methyl)benzo[b]thiophene-2-carboxylate

To a solution of ((2-((allyloxy)carbonyl)benzo[b]thiophen-5-yl)methyl)phosphonic acid (49.3 mg, 158 μmol, 1 eq.), propan-2-yl (2S)-2-amino3-methoxypropanoate hydrochloride (31.2 mg, 158 μmol, 1 eq.) and phenol (19.3 mg, 206 μmol, 1.3 eq.) in pyridine (3 mL) were added N,N-diisopropylethylamine (162 mg, 1.26 mmol, 8 eq.), 2,2-dipyridyl-disulfide (140 mg, 635 μmol, 4 eq.) and triphenylphosphine (166 mg, 635 μmol, 4 eq.) at 25° C. The mixture was heated to 60° C. and stirred at 60° C. for 48 h under N₂ atmosphere. After completion, the reaction mixture was concentrated under reduced pressure. The residue was purified by Biotage® C18 column chromatography to afford allyl 5-(((((S)-1-isopropoxy-3-methoxy-1-oxopropan-2-yl)amino)(phenoxy)phosphoryl)methyl)benzo[b]thiophene-2-carboxylate (35.0 mg, 65.8 μmol, 42%) as a yellow solid. LCMS (ESI): m/z=532 [M+H]⁺.

The following intermediates in Table 66 were prepared using the method described above in step 1 for the preparation of allyl 5-(((((S)-1-isopropoxy-3-methoxy-1-oxopropan-2-yl)amino)(phenoxy)phosphoryl)methyl)benzo[b]thiophene-2-carboxylate and utilizing the appropriate starting materials and modifications.

TABLE 66

| Name | Structure | LCMS |
|---|---|---|
| allyl 5-((((1-isopropoxy-2-methyl-1- oxopropan-2-yl)amino)(phenoxy) phosphoryl)methyl)benzo[b]thiophene- 2-carboxylate | 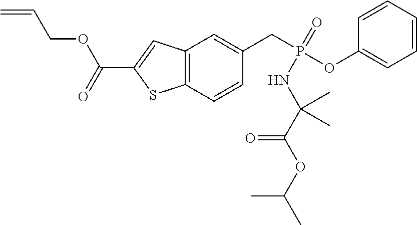 [SMILES: C=CCOC(=O)c1cc2cc(CP(=O)(NC(C)(C)C(=O)OC(C)C)Oc3ccccc3)ccc2s1] | 516.2 [M + H]⁺ |
| diisopropyl 2,2′-((((2-((allyloxy)carbonyl) benzo[b]thiophen-5- yl)methyl)phosphoryl)bis(azanediyl)) (2S,2′S)-dipropionate | 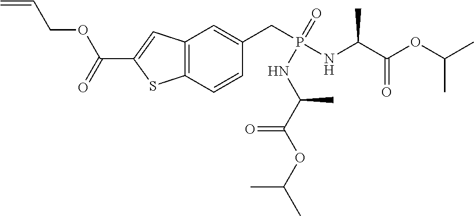 [SMILES: C=CCOC(=O)c1cc2cc(CP(=O)(N[C@@H](C)C(=O)OC(C)C)N[C@@H](C)C(=O)OC(C)C)ccc2s1] | 539 [M + H]⁺ |
| allyl 5-((((2-methyl-1-oxo-1- propoxypropan-2- yl)amino)(phenoxy)phosphoryl) methyl)benzo[b]thiophene- 2-carboxylate | 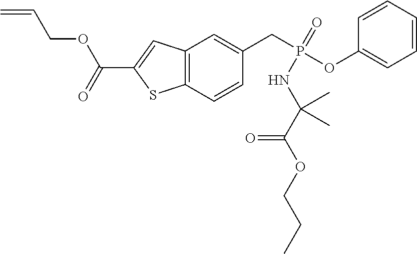 [SMILES: C=CCOC(=O)c1cc2cc(CP(=O)(NC(C)(C)C(=O)OCCC)Oc3ccccc3)ccc2s1] | 516.1 [M + H]⁺ |

### Step 2: Preparation of 5-(((((S)-1-isopropoxy-3-methoxy-1-oxopropan-2-yl)amino)(phenoxy)phosphoryl)methyl)benzo[b]thiophene-2-carboxylic acid

To a solution of allyl 5-(((((S)-1-isopropoxy-3-methoxy-1-oxopropan-2-yl)amino)(phenoxy)phosphoryl)methyl)benzo[b]thiophene-2-carboxylate (2, 150 mg, 282 μmol, 1 eq) in DCM (5 mL) were added Pd(PPh₃)₄(65.1 mg, 56.4 μmol, 0.2 eq.) and pyrrolidine (20.0 mg, 282 μmol, 1.0 eq.). The mixture was purged and degassed with N₂ (three times), then stirred at 25° C. for 1 hr. After completion, the mixture was concentrated under reduced pressure. The residue was purified by Biotage® C18 column chromatography to give 5-(((((S)-1-isopropoxy-3-methoxy-1-oxopropan-2-yl)amino)(phenoxy)phosphoryl)methyl)benzo[b]thiophene-2-carboxylic acid (120 mg, 244 μmol, 87%) as a yellow solid. LCMS (ESI): m/z=492.1 [M+H]⁺.

The following intermediates in Table 67 were prepared using the method described above in step 2 for the preparation of 5-(((((S)-1-isopropoxy-3-methoxy-1-oxopropan-2-yl)amino)(phenoxy)phosphoryl)methyl)benzo[b]thiophene-2-carboxylic acid and utilizing the appropriate starting materials and modifications.

TABLE 67

| Name | Structure | LCMS |
|---|---|---|
| 5-((((1-isopropoxy-2-methyl-1-oxopropan-2- yl)amino)(phenoxy)phosphoryl)methyl)benzo [b]thiophene-2-carboxylic acid | 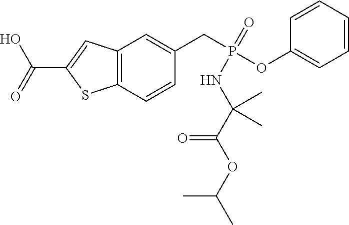 [SMILES: CC(C)OC(=O)C(C)(C)NP(=O)(Cc1ccc2sc(C(=O)O)cc2c1)Oc1ccccc1] | 476.5 [M + H]⁺ |
| 5-((bis(((S)-1-isopropoxy-1-oxopropan-2- yl)amino)phosphoryl)methyl)benzo[b] thiophene-2-carboxylic acid | 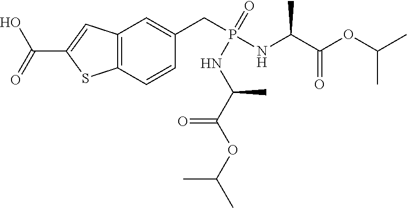 [SMILES: CC(C)OC(=O)[C@H](C)NP(=O)(Cc1ccc2sc(C(=O)O)cc2c1)N[C@@H](C)C(=O)OC(C)C] | 499 [M + H]⁺ |
| 5-((((2-methyl-1-oxo-1-propoxypropan-2- yl)amino)(phenoxy)phosphoryl)methyl)benzo [b]thiophene-2-carboxylic acid | 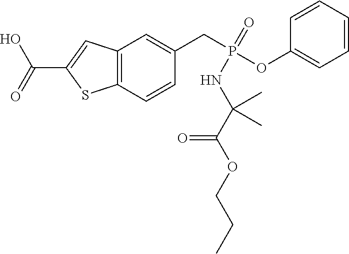 [SMILES: CCCOC(=O)C(C)(C)NP(=O)(Cc1ccc2sc(C(=O)O)cc2c1)Oc1ccccc1] | 476.2 [M + H]⁺ |

### Synthesis of 5-(1-fluoro-1-((((S)-1-oxo-1-propoxypropan-2-yl)amino)(phenoxy)phosphoryl)ethyl)benzo[b]thiophene-2-carboxylic acid

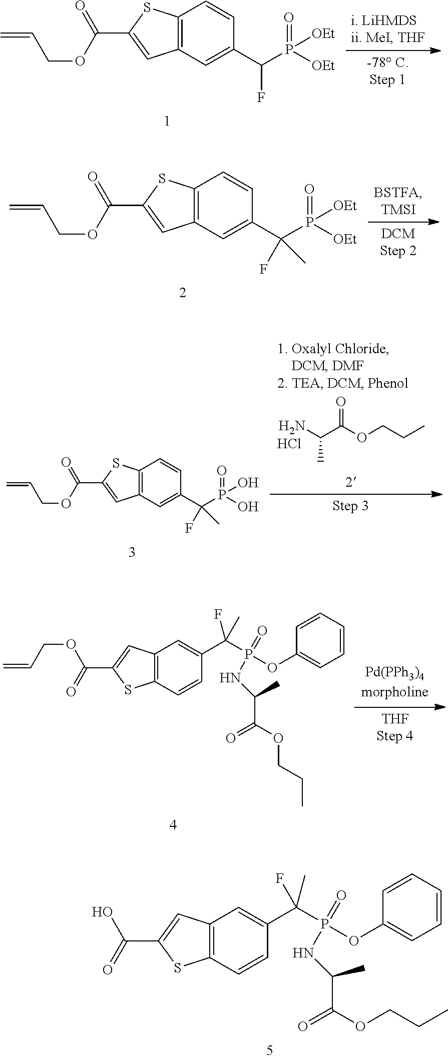

### Step 1: Preparation of Allyl 5-(1-(diethoxyphosphoryl)-1-fluoroethyl)benzo[b]thiophene-2-carboxylate

To a solution of allyl 5-((diethoxyphosphoryl)fluoromethyl)benzo[b]thiophene-2-carboxylate (2.66 g, 6.88 mmol, 1 eq) in THF (50 mL) at −78° C. were added 1 M LiHMDS in THF (6.88 mL, 6.88 mmol, 1 eq) and methyl iodide (850 μL, 13.7 mmol, 2 eq) dropwise. The mixture was stirred at −78° C. for 5 min. The reaction was quenched with a saturated aqueous solution of ammonium chloride (30 mL), warmed up to room temperature and the product was extracted with EtOAc (2×100 mL). The combined organic extracts were dried with sodium sulfate, filtered and concentrated under reduced pressure. The crude material was purified by reverse phase chromatography on a 150 g C₁₈ cartridge eluting with a gradient of 5-80% acetonitrile in water (with 0.1% formic acid). The fractions were combined and concentrated under reduced pressure to give allyl 5-(1-(diethoxyphosphoryl)-1-fluoroethyl)benzo[b]thiophene-2-carboxylate (1.44 g, 52.3%) as a brown oil. LCMS (ESI) m/z=401.2

### Step 2: Preparation of (1-(2-((allyloxy)carbonyl)benzo[b]thiophen-5-yl)-1-fluoroethyl)phosphonic acid

To a solution of allyl 5-(1-(diethoxyphosphoryl)-1-fluoroethyl)benzo[b]thiophene-2-carboxylate (1.44 g, 3.59 mmol, 1 eq) in DCM (30 mL) at 0° C. were added N,O-bis(trimethylsilyl)trifluoroacetamide (4.74 mL, 17.9 mmol) followed by slow addition of trimethylsilyl iodide (1.52 mL, 10.7 mmol, 3 eq) in DCM (2 mL). The solution was stirred at 0° C. for 1 h. The reaction was quenched with the addition of a 2:1 solution of water and acetonitrile (with 0.1% TFA) (2 mL). The solvent was removed under reduced pressure. The crude residue was purified by reverse phase chromatography on a 150 g C₁₈ cartridge eluting with a gradient of 5-60% acetonitrile in water (with 0.1% formic acid). The combined fractions were concentrated and freeze-dried to give (1-(2-((allyloxy)carbonyl)benzo[b]thiophen-5-yl)-1-fluoroethyl)phosphonic acid (887 mg, 72.1%) as a pale-yellow solid. ¹H NMR (400 MHz, DMSO-d₆) 11.84-11.05 (m, 2H), 8.34-8.28 (m, 1H), 8.12-8.02 (m, 2H), 7.66-7.56 (m, 1H), 6.12-5.98 (m, 1H), 5.49-5.39 (m, 1H), 5.35-5.27 (m, 1H), 4.89-4.81 (m, 2H), 1.96-1.78 (m, 3H).

### Step 3: Preparation of Allyl 5-(1-fluoro-1-((((S)-1-oxo-1-propoxypropan-2-yl)amino)(phenoxy)phosphoryl)ethyl)benzo[b]thiophene-2-carboxylate

To a suspension of (1-(2-((allyloxy)carbonyl)benzo[b]thiophen-5-yl)-1-fluoroethyl)phosphonic acid (160 mg, 464 μmol, 1 eq) and 1 drop of DMF (cat) in DCM (30 mL) at 0° C. was added oxalyl chloride (118 μL, 1.39 mmol, 3 eq) and the reaction was stirred for 20 h. The reaction was concentrated under reduced pressure and then dried under high vacuum during 10 min. The crude residue was diluted in 1,2-dichloroethane (3 mL) and a solution of phenol (43.6 mg, 464 μmol, 1 eq) and triethylamine (322 μL, 2.32 mmol, 5 eq) in 1,2-dichloroethane (3 mL, dried with Na₂SO₄) was added to the mixture which was stirred at 45° C. for 3 h. Propyl (2S)-2-aminopropanoate hydrochloride (77.7 mg, 464 μmol, 1 eq) in dichloromethane (10 mL, dried with Na₂SO₄) was added directly to the solution and stirred at room temperature overnight. The reaction mixture was quenched with water and the dichloromethane was removed in vacuo. The product was purified by reverse phase chromatography on a 50 g C₁₈ cartridge eluting with a gradient of 5-100% acetonitrile in water (with 0.1% formic acid) to afford allyl 5-(1-fluoro-1-((((S)-1-oxo-1-propoxypropan-2-yl)amino)(phenoxy)phosphoryl)ethyl)benzo[b]thiophene-2-carboxylate (30.0 mg, 12%). ¹H NMR (400 MHz, CDCl₃) δ 8.12-8.02 (m, 2H), 7.93-7.82 (m, 1H), 7.72-7.64 (m, 1H), 7.38-7.28 (m, 1H), 7.25-7.13 (m, 2H), 7.09-7.00 (m, 1H), 7.00-6.92 (m, 1H), 6.13-5.98 (m, 1H), 5.45 (br d, J=17.1 Hz, 1H), 5.33 (br d, J=10.5 Hz, 1H), 4.86 (br d, J=4.9 Hz, 2H), 4.25-4.11 (m, 0.5H), 4.10-4.00 (m, 1H), 3.95-3.76 (m, 1H), 3.68-3.48 (m, 1.25H), 3.45-3.36 (m, 0.25H), 2.44-2.74 (m, 1H), 2.17-2.01 (m, 3H), 1.70-1.59 (m, 1H), 1.56-1.46 (m, 0.5H), 1.45-1.33 (m, 0.5H), 1.32-1.23 (m, 1.5H), 1.13-1.05 (m, 0.5H), 0.97-0.89 (m, 1.5H), 0.87-0.80 (m, 0.5H), 0.80-0.72 (m, 1H).

### Step 4: Preparation of 5-(1-fluoro-1-((((S)-1-oxo-1-propoxypropan-2-yl)amino)(phenoxy)phosphoryl)ethyl)benzo[b]thiophene-2-carboxylic acid

To a stirred solution of allyl 5-(1-fluoro-1-((((S)-1-oxo-1-propoxypropan-2-yl)amino)(phenoxy)phosphoryl)ethyl)benzo[b]thiophene-2-carboxylate (30 mg, 56.2 μmol, 1 eq) in THF (3 mL) were added morpholine (24.1 μL, 281 μmol, 5 eq) and Pd(PPh₃)₄ (3.24 mg, 2.81 μmol, 0.05 eq) under nitrogen. The reaction mixture was stirred at room temperature for 1 h. The reaction was concentrated and the crude was directly purified by reverse phase chromatography on a 50 g C₁₈ cartridge eluting with a gradient of 5-100% acetonitrile in water (with 0.1% formic acid) to afford 5-(1-fluoro-1-((((S)-1-oxo-1-propoxypropan-2-yl)amino)(phenoxy)phosphoryl)ethyl)benzo[b]thiophene-2-carboxylic acid (30.0 mg, 108%) as a beige solid. LCMS (ESI) m/z=494.2 [M+H]⁺.

### Synthesis of perfluorophenyl 5-(((((S)-1-oxo-1-propoxypropan-2-yl)amino)(phenoxy)phosphoryl)methyl)benzo[b]thiophene-2-carboxylate

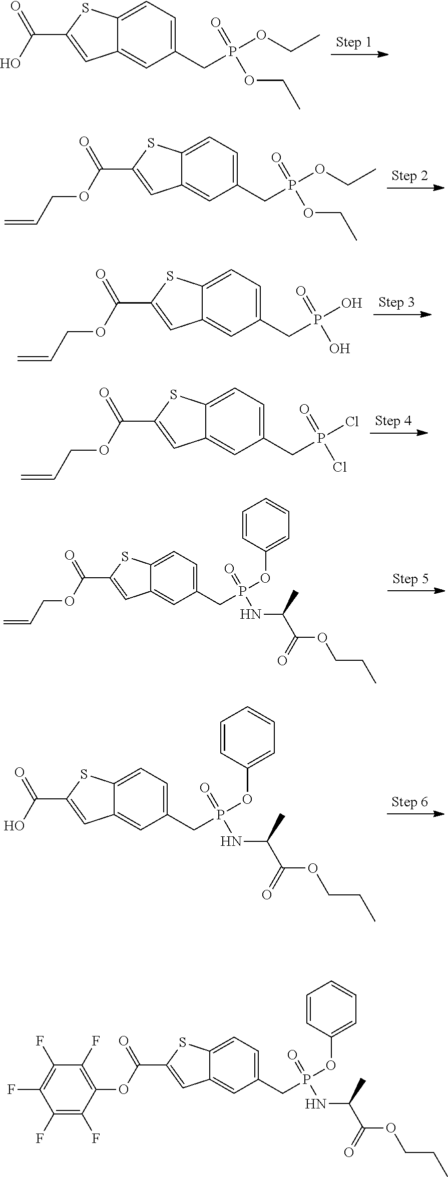

### Step 1: Preparation of Allyl 5-((diethoxyphosphoryl)methyl)benzo[b]thiophene-2-carboxylate

To a solution of 5-((diethoxyphosphoryl)methyl)benzo[b]thiophene-2-carboxylic acid (25 g, 76 mmol, 1 eq) in DMF (250 mL) was added Na₂CO₃ (20 g, 190 mmol, 2.5 eq) and 3-bromoprop-1-ene (23 g, 190 mmol, 2.5 eq), the mixture was stirred at 25° C. for 12 h. The mixture was diluted with water (500 mL) and extracted with EtOAc (500 mL×3). The combined organic layers were washed with brine (500 mL×3), dried over anhydrous Na₂SO₄, filtered and concentrated under reduced pressure to give a residue. The residue was purified by flash silica gel chromatography to give allyl 5-((diethoxyphosphoryl)methyl)benzo[b]thiophene-2-carboxylate (25 g, 68 mmol, 89% yield) as a white solid. ¹H NMR (400 MHz, CDCl₃) δ 7.97 (s, 1H), 7.79-7.67 (m, 2H), 7.39-7.28 (m, 1H), 6.05-5.88 (m, 1H), 5.43-5.33 (m, 1H), 5.29-5.20 (m, 1H), 4.83-4.73 (m, 2H), 4.00-3.88 (m, 4H), 3.29-3.11 (m, 2H), 1.18 (d, J=6.4 Hz, 6H)

### Step 2: Preparation of ((2-((allyloxy)carbonyl)benzo[b]thiophen-5-yl)methyl)phosphonic acid

To a solution of allyl 5-((diethoxyphosphoryl)methyl)benzo[b]thiophene-2-carboxylate (25 g, 68 mmol, 1 eq) in DCM (250 mL) was added TMSI (54 g, 272 mmol, 4 eq) in a dropwise manner at 0° C., the mixture was stirred at 0° C. for 1 h. The reaction mixture was concentrated under reduced pressure to give a yellow oil. The oil was purified by reversed phase (TFA) to lyophilized to give ((2-((allyloxy)carbonyl)benzo[b]thiophen-5-yl)methyl)phosphonic acid (13 g, 42 mmol, 61% yield) as a white solid. LCMS (ESI) m/z=313.2 [M+H]⁺.

### Step 3: Preparation of Allyl 5-((dichlorophosphoryl)methyl)benzo[b]thiophene-2-carboxylate

To a solution of ((2-((allyloxy)carbonyl)benzo[b]thiophen-5-yl)methyl)phosphonic acid (2.0 g, 6.4 mmol, 1 eq) in DCM (30 mL) was added DMF (0.09 mL) and was cooled to 0° C., then oxalic dichloride (2.4 g, 19.2 mmol, 3 eq) was dropwise, after that the reaction was warmed to 40° C. and was stirred 1 h to give a yellow clean solution. The reaction mixture was concentrated under reduced pressure to give allyl 5 ((dichlorophosphoryl)methyl)benzo[b]thiophene-2-carboxylate (2.0 g, crude) as a brassy yellow solid. LCMS (ESI) m/z=341.2 [M+H]⁺.

### Step 4: Preparation of Allyl 5-(((((S)-1-oxo-1-propoxypropan-2-yl)amino)(phenoxy)phosphoryl)methyl)benzo[b]thiophene-2-carboxylate

To a solution of allyl 5-((dichlorophosphoryl)methyl)benzo[b]thiophene-2-carboxylate (2.0 g, crude, 1 eq) in DCM (40 mL) was cooled to 0° C. and was added a solution of phenol (0.43 g, 4.58 mmol, 1 eq) in DCM (8 mL), then ethylbis(propan-2-yl)amine (2.2 g, 17 mmol, 3 eq) in DCM (50 mL) was dropwise over 0.5 h. Then propyl L-alaninate hydrochloride (1.44 g, 1.58 mmol, 1.5 eq) in DCM (8 mL) was added, the mixture was stirred at 0° C. for 0.5 h. The reaction mixture was concentrated under reduced pressure to give a residue. The residue was purified by column chromatography to give allyl 5-(((((S)-1-oxo-1-propoxypropan-2-yl)amino)(phenoxy)phosphoryl)methyl)benzo[b]thiophene-2-carboxylate (1.4 g, 2.8 mmol, 43.8% yield) as a yellow oil. LCMS (ESI) m/z=502.2 [M+H]⁺.

### Step 5: Preparation of 5-(((((S)-1-oxo-1-propoxypropan-2-yl)amino)(phenoxy)phosphoryl)methyl)benzo[b]thiophene-2-carboxylic acid

To a solution of allyl 5-(((((S)-1-oxo-1-propoxypropan-2-yl)amino)(phenoxy)phosphoryl)methyl)benzo[b]thiophene-2-carboxylate (1.4 g, 2.8 mmol) in DCM (20 mL) was added Pd(PPh₃)₄(0.3 g, 0.3 mmol, 0.1 eq) and pyrrolidine (0.4 g, 5.6 mmol, 2 eq). Then the reaction was stirred at 25° C. for 15 min to give a yellow clean solution. The reaction mixture was concentrated under reduced pressure to give a residue. The residue was purified by reversed phase (TFA) to freeze-drying to give 5-(((((S)-1-oxo-1-propoxypropan-2-yl)amino)(phenoxy)phosphoryl)methyl)benzo[b]thiophene-2-carboxylic acid (1.1 g, 2.4 mmol, 85.9% yield) as a yellow oil. LCMS (ESI) m/z=461.9 [M+H]⁺. ¹H NMR (400 MHz, CDCl₃) δ 7.91-7.86 (m, 1H), 7.84-7.76 (m, 2H), 7.45-7.38 (m, 1H), 7.38-7.31 (m, 2H), 7.27-7.15 (m, 3H), 4.28-3.95 (m, 3H), 3.56-3.42 (m, 2H), 1.64 (td, J=7.2, 14.0 Hz, 2H), 1.33-1.18 (m, 3H), 0.93 (dt, J=4.8, 7.2 Hz, 3H)

### Step 6: Preparation of Perfluorophenyl 5-(((((S)-1-oxo-1-propoxypropan-2-yl)amino)(phenoxy)phosphoryl)methyl)benzo[b]thiophene-2-carboxylate

To a solution of 5-(((((S)-1-oxo-1-propoxypropan-2-yl)amino)(phenoxy)phosphoryl)methyl)benzo[b]thiophene-2-carboxylic acid (19 g, 41 mmol, 1 eq) and pyridine (11 g, 0.14 mol, 3.5 eq) in DMF (100 mL) was cooled to 0° C., then the solution of perfluorophenyl 2,2,2-trifluoroacetate (57 g, 0.21 mol, 5 eq) was dropwise added to the mixture at 0° C. Then the mixture was stirred at 0° C. for 1 h to give a yellow solution. The mixture was diluted with water (500 mL) and extracted with EtOAc (500 mL×3), the combined organic layers were dried over Na₂SO₄, filtered and concentrated under reduced pressure to give a residue. The residue was purified by flash silica gel chromatography to give perfluorophenyl 5-(((((S)-1-oxo-1-propoxypropan-2-yl)amino)(phenoxy)phosphoryl)methyl)benzo[b]thiophene-2-carboxylate (17 g, 27 mmol, 65% yield) was obtained as a yellow oil. LCMS (ESI) m/z=628.1 [M+H]⁺. ¹H NMR (400 MHz, CDCl₃) δ 8.33-8.23 (m, 1H), 8.03-7.85 (m, 2H), 7.60-7.53 (m, 1H), 7.28 (s, 2H), 7.20-7.05 (m, 3H), 4.08-3.87 (m, 3H), 3.55-3.42 (m, 2H), 1.63-1.52 (m, 2H), 1.19-1.15 (m, 3H), 0.95-0.83 (m, 3H).

### Synthesis of perfluorophenyl 5-((S)-fluoro((R)-(((S)-1-oxo-1-propoxypropan-2-yl)amino)(phenoxy)phosphoryl)methyl)benzo[b]thiophene-2-carboxylate

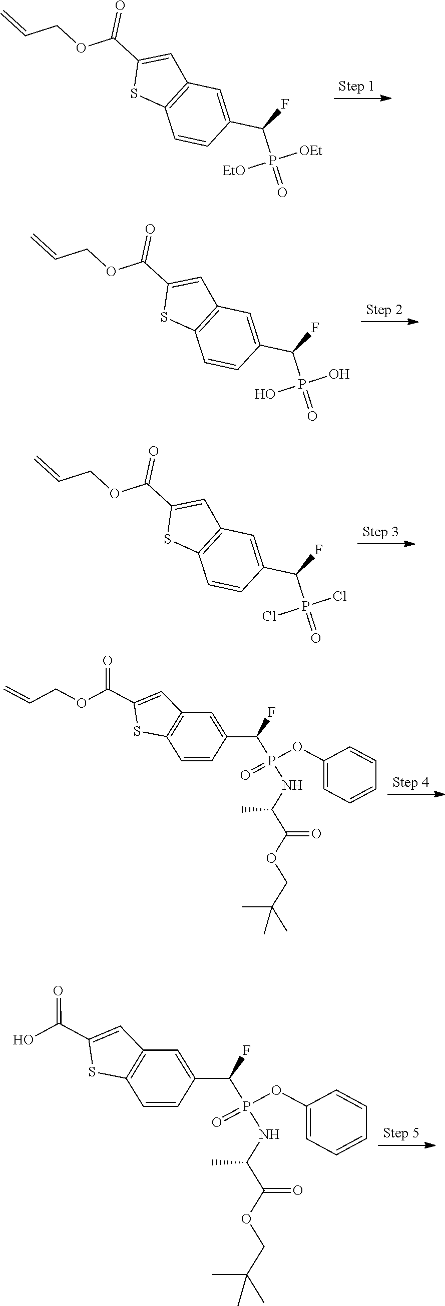 [SMILES: C=CCOC(=O)c1cc2cc([C@@H](F)P(=O)(OCC)OCC)ccc2s1 ; C=CCOC(=O)c1cc2cc([C@@H](F)P(=O)(O)O)ccc2s1 ; C=CCOC(=O)c1cc2cc([C@@H](F)P(=O)(Cl)Cl)ccc2s1 ; C=CCOC(=O)c1cc2cc([C@@H](F)P(=O)(N[C@@H](C)C(=O)OCC(C)(C)C)Oc3ccccc3)ccc2s1 ; C[C@H](NP(=O)(Oc1ccccc1)[C@H](F)c1ccc2sc(C(=O)O)cc2c1)C(=O)OCC(C)(C)C]

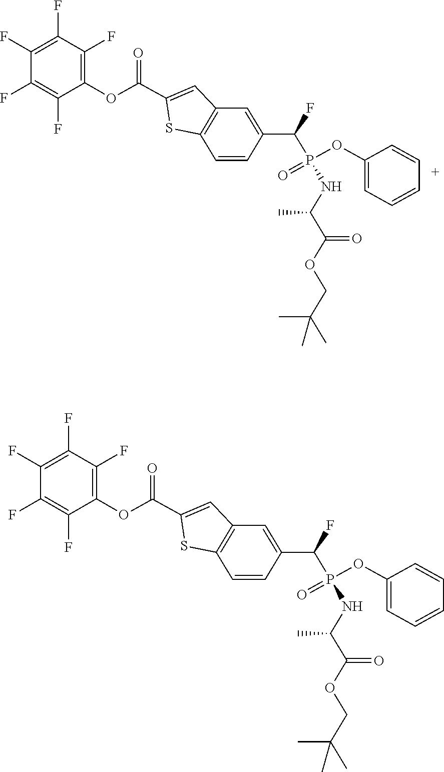 [SMILES: C[C@H](N[P@@](=O)(Oc1ccccc1)[C@H](F)c1ccc2sc(C(=O)Oc3c(F)c(F)c(F)c(F)c3F)cc2c1)C(=O)OCC(C)(C)C ; C[C@H](N[P@](=O)(Oc1ccccc1)[C@H](F)c1ccc2sc(C(=O)Oc3c(F)c(F)c(F)c(F)c3F)cc2c1)C(=O)OCC(C)(C)C]

### Step 1: Preparation of (S)-((2-((allyloxy)carbonyl)benzo[b]thiophen-5-yl)fluoromethyl)phosphonic acid

To a solution of allyl (S)-5-((diethoxyphosphoryl)fluoromethyl)benzo[b]thiophene-2-carboxylate (20 g, 52 mmol, 1 eq) in methylene chloride (500 mL) was added trimethylsilyl iodide (21 g, 0.10 mol, 2 eq). The mixture was stirred at 0° C. for 1 hour to give a brown solution. The reaction mixture was concentrated under reduced pressure to give a residue. The reaction residue was purified by prep-HPLC (TFA) to lyophilized to give (S)-((2-((allyloxy)carbonyl)benzo[b]thiophen-5-yl)fluoromethyl)phosphonic acid (12 g, 36 mmol, 70% yield) as a brown solid. LCMS (ESI) m/z=330.9

### Step 2: Preparation of allyl (S)-5-((dichlorophosphoryl)fluoromethyl)benzo[b]thiophene-2-carboxylate

To a solution of (S)-((2-((allyloxy)carbonyl)benzo[b]thiophen-5-yl)fluoromethyl)phosphonic acid (11 g, 33 mmol, 1 eq) in methylene chloride (200 mL) was added dimethylformamide (0.24 g, 3.3 mmol, 0.1 eq) at 0° C. under N₂ atmosphere, then oxalyl chloride (13 g, 0.10 mol, 3 eq) was added dropwise and stirred at 0° C. for 30 minutes and warmed to 40° C. for 1 hour to give a brown solution. The reaction mixture was concentrated under reduced pressure to give allyl (S)-5-((dichlorophosphoryl)fluoromethyl)benzo[b]thiophene-2-carboxylate (11 g crude) as a yellow solid. LCMS (ESI) m/z=358.9.

### Step 3: Preparation of Allyl 5-((1S)-fluoro((((S)-1-oxo-1-propoxypropan-2-yl)amino)(phenoxy)phosphoryl)methyl)benzo[b]thiophene-2-carboxylate

To a solution of (S)-5-((dichlorophosphoryl)fluoromethyl)benzo[b]thiophene-2-carboxylate (11 g, 30 mmol, 1 eq) in methylene chloride (150 mL) was added phenol (2.2 g, 24 mmol, 0.8 eq) in methylene chloride (20 mL) in 10 minutes at 0° C., then the solution of N,N-diisopropylethylamine (12 g, 90 mmol, 3 eq) in methylene chloride (200 mL) was dropwise over 1.5 hours and the mixture was stirred at 25° C. for 5 minutes to give a yellow clean solution then the solution of propyl (2S)-2-aminopropanoate (3.9 g, 30 mmol, 1 eq) in methylene chloride (20 mL) was added, the mixture was stirred at 25° C. for 1 hour to give a yellow clean solution. The reaction mixture was concentrated under reduced pressure to give a residue. The residue was purified by flash silica gel chromatography to give allyl 5-((1S)-fluoro((((S)-1-oxo-1-propoxypropan-2-yl)amino)(phenoxy)phosphoryl)methyl)benzo[b]thiophene-2-carboxylate (7.4 g, 14.2 mmol, 47% yield) as a yellow solid. LCMS (ESI) m/z=520.2

### Step 4: Preparation of 5-((1S)-fluoro((((S)-1-oxo-1-propoxypropan-2-yl)amino)(phenoxy)phosphoryl)methyl)benzo[b]thiophene-2-carboxylic acid

To a solution of allyl 5-((1S)-fluoro((((S)-1-oxo-1-propoxypropan-2-yl)amino)(phenoxy)phosphoryl)methyl)benzo[b]thiophene-2-carboxylate (3.5 g, 6.7 mmol, 1 eq) in methylene chloride (40 mL) at 0° C. under N₂ atmosphere, then pyrrolidine (0.38 g, 5.4 mmol, 0.8 eq) and Pd(PPh₃)₄(0.7 g, 0.67 mmol, 0.11 eq) was added dropwise and stirred at 0° C. for 10 minutes and warmed to 25° C. for 10 minutes to give a brown solution. The reaction mixture was concentrated under reduced pressure to give a residue. The reaction residue was purified by prep-HPLC (TFA) to lyophilized to give 5-((1S)-fluoro((((S)-1-oxo-1-propoxypropan-2-yl)amino)(phenoxy)phosphoryl)methyl)benzo[b]thiophene-2-carboxylic acid (2.8 g, 5.9 mmol, 87% yield) as a white solid. ¹H NMR (400 MHz, CDCl₃) δ 8.10 (d, J=11.2 Hz, 2H), 7.97 (d, J=8.4 Hz, 1H), 7.66 (d, J=8.4 Hz, 1H), 7.38-7.30 (m, 2H), 7.19 (d, J=7.6 Hz, 3H), 6.20-5.99 (m, 1H), 4.05-3.77 (m, 3H), 1.68-1.42 (m, 2H), 1.22 (d, J=7.2 Hz, 3H), 0.89-0.85 (m, 3H)

### Step 5: Preparation of Perfluorophenyl 5-((S)-fluoro((R)-(((S)-1-oxo-1-propoxypropan-2-yl)amino)(phenoxy)phosphoryl)methyl)benzo[b]thiophene-2-carboxylate and perfluorophenyl 5-((S)-fluoro((S)-(((S)-1-oxo-1-propoxypropan-2-yl)amino)(phenoxy)phosphoryl)methyl)benzo[b]thiophene-2-carboxylate

To a solution of 5-((1S)-fluoro((((S)-1-oxo-1-propoxypropan-2-yl)amino)(phenoxy)phosphoryl)methyl)benzo[b]thiophene-2-carboxylic acid (3.0 g, 6.3 mmol, 1 eq) in pyridine (15 mL) at 0° C. under N₂ atmosphere, then 2,3,4,5,6-pentafluorophenyl 2,2,2-trifluoroacetate (5.2 g, 19 mmol, 3 eq) was added dropwise and stirred at 0° C. for 10 minutes and warmed to 25° C. for 1 hour to give a brown solution. The reaction mixture was concentrated under reduced pressure to give a residue. The residue was purified by flash silica gel chromatography in 3:1 Petroleum ether/ethyl acetate to give the separated Phos isomers. The analogous (R)—F isomer was made in a similar manner. Analytical data for all 4 isomers are listed in the table below. SFC conditions used in the peak assignments are as follows: Chiralpak AS-3 50×4.6 mm I.D., 3 um. Mobile phase: Phase A for CO2, and Phase B for EtOH (0.05% DEA); Gradient elution: B in A from 5% to 40%. Flow rate: 3 mL/min; Detector: PDA; Column Temp: 35 C; Back Pressure: 100 Bar.

| Name | Structure | LCMS | NMR |
|---|---|---|---|
| perfluorophenyl 5- ((S)-fluoro((R)-(((S)- 1-oxo-1- propoxypropan-2- yl)amino)(phenoxy) phosphoryl)methyl)benzo[b] thiophene-2-carboxylate or perfluorophenyl 5- ((S)-fluoro((S)-(((S)- 1-oxo-1- propoxypropan-2- yl)amino)(phenoxy) phosphoryl)methyl)benzo[b] thiophene-2-carboxylate | 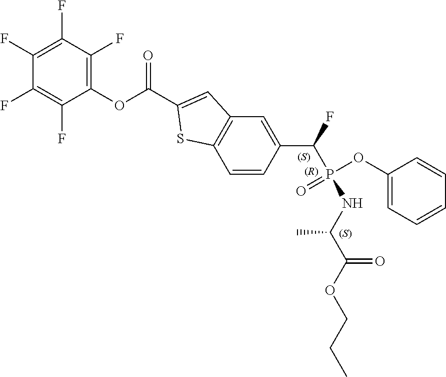 | 645.9 [M + H]⁺ | ¹H NMR (400 MHZ, CD₃OD) δ 8.52 (s, 1H), 8.25 (s, 1H), 8.14 (d, J = 8.4 Hz, 1H), 7.81 (d, J = 8.4 Hz, 1H), 7.39-7.32 (m, 2H), 7.27-7.16 (m, 3H), 6.25-6.01 (m, 1H), 4.00 (t, J = 6.8 Hz, 2H), 3.92-3.82 (m, 1H), 1.63-1.52 (m, 2H), 1.14 (d, J = 7.2 Hz, 3H), 0.90 (t, J = 7.2 Hz, 3H) |
| | 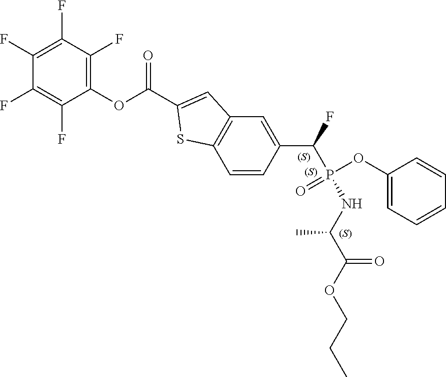 | | |
| | CFTE-27B, SFC Peak 2 Ret. Time = 1.46 Minutes | | |
| perfluorophenyl 5- ((S)-fluoro((R)-(((S)- 1-oxo-1- propoxypropan-2- yl)amino)(phenoxy) phosphoryl)methyl)benzo[b] thiophene-2- carboxylate or perfluorophenyl 5- ((S)-fluoro((S)-(((S)- 1-oxo-1- propoxypropan-2- yl)amino)(phenoxy) phosphoryl)methyl)benzo[b] thiophene-2-carboxylate | 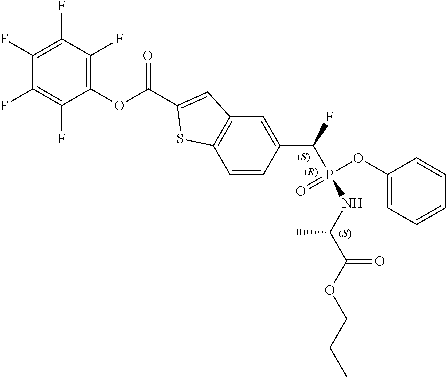 | 646.0 [M + H]⁺ | ¹H NMR (400 MHZ, CD₃OD) δ 8.51 (s, 1H), 8.27 (s, 1H), 8.11 (d, J = 8.4 Hz, 1H), 7.79 (d, J = 8.8 Hz, 1H), 7.40-7.33 (m, 2H), 7.25-7.18 (m, 3H), 6.27-6.08 (m, 1H), 4.03-3.85 (m, 3H), 1.63-1.51 (m, 2H), 1.25 (d, J = 7.2 Hz, 3H), 0.89 (t, J = 7.6 Hz, 3H) |
| | 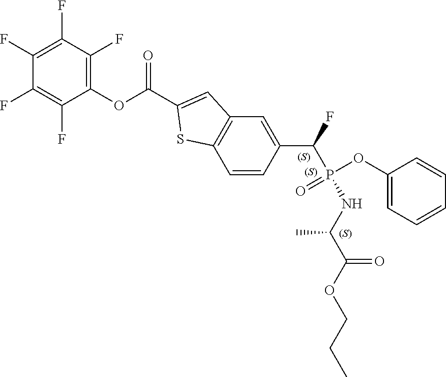 | | |
| | CFTE-27A, SFC Peak 1 Ret. Time = 0.89 Minutes | | |
| perfluorophenyl 5- ((R)-fluoro((R)-(((S)- 1-oxo-1- propoxypropan-2- yl)amino)(phenoxy) phosphoryl)methyl) benzo[b]thiophene-2- carboxylate or perfluorophenyl 5- ((R)-fluoro((S)-(((S)- 1-oxo-1- propoxypropan-2- yl)amino)(phenoxy) phosphoryl)methyl) benzo[b]thiophene-2- carboxylate | 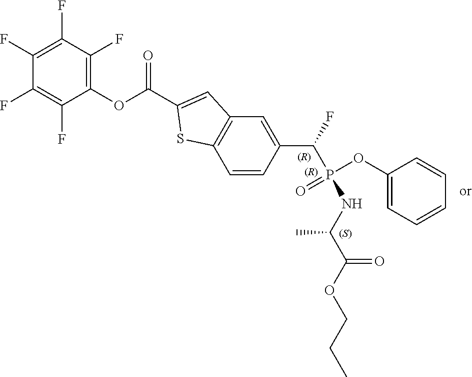 | 645.9 [M + H]⁺ | ¹H NMR (400 MHZ, CD₃OD) δ 8.50-8.44 (m, 1H), 8.26-8.18 (m, 1H), 8.13-8.03 (m, 1H), 7.81-7.72 (m, 1H), 7.41-7.28 (m, 2H), 7.27-7.11 (m, 3H), 6.22-6.00 (m, 1H), 4.01-3.94 (m, 2H), 3.93-3.83 (m, 1H), 1.63-1.51 (m, 2H), 1.23 (d, J = 7.2 Hz, 3H), 0.93- 0.83 (m, 3H) |
| | 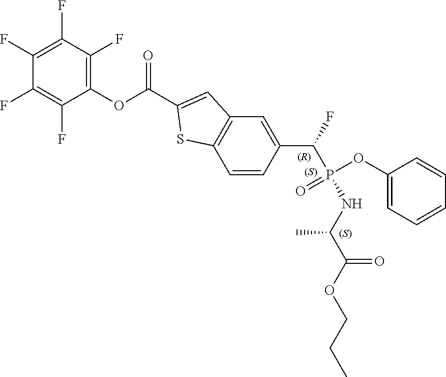 | | |
| | CFTE-28A, SFC Peak 2 Ret. Time = 1.95 Minutes | | |
| perfluorophenyl 5- ((R)-fluoro((R)-(((S)- 1-oxo-1- propoxypropan-2- yl)amino)(phenoxy) phosphoryl)methyl) benzo[b]thiophene-2- carboxylate or perfluorophenyl 5- ((R)-fluoro((S)-(((S)- 1-oxo-1- propoxypropan-2- yl)amino)(phenoxy) phosphoryl)methyl) benzo[b]thiophene-2-carboxylate | 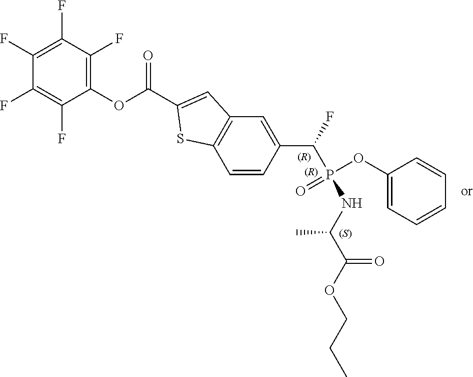 | 646.1 [M + H]⁺ | ¹H NMR (400 MHZ, METHANOL-d4) δ 8.40 (s, 1H), 8.17- 8.06 (m, 1H), 8.04- 7.95 (m, 1H), 7.75- 7.64 (m, 1H), 7.29- 7.18 (m, 2H), 7.14- 6.98 (m, 3H), 6.18- 5.93 (m, 1H), 3.87- 3.79 (m, 1H), 3.79- 3.67 (m, 2H), 1.48- 1.36 (m, 2H), 1.15- 1.09 (m, 3H), 0.76 (t, J = 7.6 Hz, 3H) |
| | 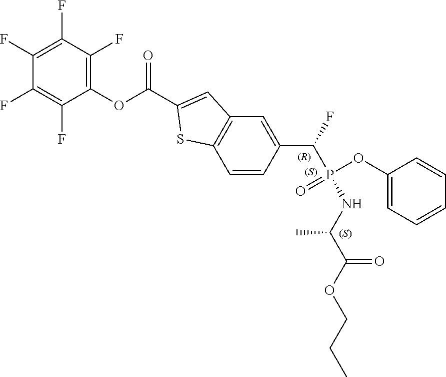 | | |
| | CFTE-28B, SFC Peak 1 Ret. Time = 1.51 Minutes | | |

The following intermediates in Table 68 were prepared using the synthetic procedure described above Synthesis of perfluorophenyl 5-((S) fluoro((R)-(((S)-1-oxo-1-propoxypropan-2-yl)amino)(phenoxy)phosphoryl)methyl)benzo[b]thiophene-2-carboxylate and utilizing the appropriate starting materials and modifications.

TABLE 68

| Name | Structure | LCMS | NMR |
|---|---|---|---|
| perfluorophenyl 5- ((S)-fluoro((R)-(((S)- 1-isopropoxy-1- oxopropan-2- yl)amino)(phenoxy) phosphoryl)methyl) benzo[b]thiophene-2- carboxylate or perfluorophenyl 5- ((S)-fluoro((S)-(((S)-1- isopropoxy-1- oxopropan-2- yl)amino)(phenoxy) phosphoryl)methyl) benzo[b]thiophene-2- carboxylate | 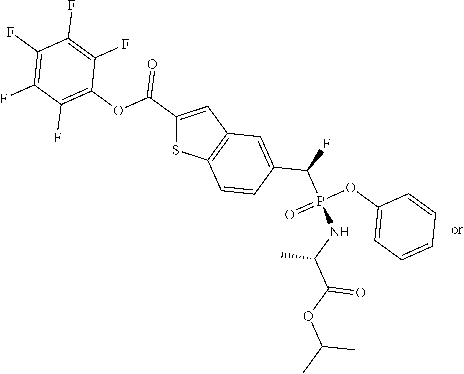 [SMILES: CC(C)OC(=O)[C@H](C)N[P@](=O)(Oc1ccccc1)[C@H](F)c1ccc2sc(C(=O)Oc3c(F)c(F)c(F)c(F)c3F)cc2c1] | 646.2 [M + H]⁺ | |
| | 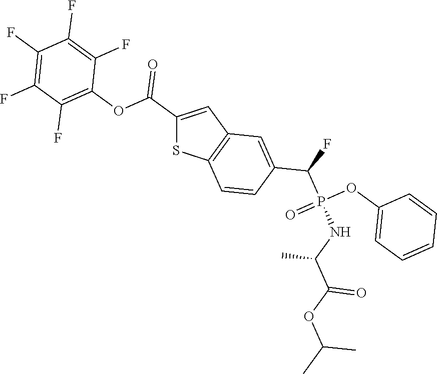 [SMILES: CC(C)OC(=O)[C@H](C)N[P@@](=O)(Oc1ccccc1)[C@H](F)c1ccc2sc(C(=O)Oc3c(F)c(F)c(F)c(F)c3F)cc2c1] | | |
| perfluorophenyl 5- ((S)-fluoro((R)-(((S)- 1-isopropoxy-1- oxopropan-2- yl)amino)(phenoxy) phosphoryl)methyl) benzo[b]thiophene-2- carboxylate or perfluorophenyl 5- ((S)-fluoro((S)-(((S)-1- isopropoxy-1- oxopropan-2- yl)amino)(phenoxy) phosphoryl)methyl) benzo[b]thiophene-2- carboxylate | 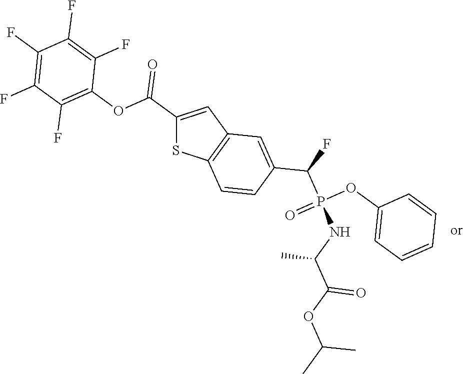 [SMILES: CC(C)OC(=O)[C@H](C)N[P@](=O)(Oc1ccccc1)[C@H](F)c1ccc2sc(C(=O)Oc3c(F)c(F)c(F)c(F)c3F)cc2c1] | 646.2 [M + H]⁺ | ¹H NMR (400 MHZ, CDCl₃) δ 8.36 (s, 1H), 8.14 (s, 1H), 8.02-7.95 (m, 1H), 7.76 (d, J = 8.4 Hz, 1H), 7.32-7.28 (m, 2H), 7.18-7.14 (m, 1H), 7.12 (d, J = 8.0 Hz, 2H), 6.10-5.94 (m, 1H), 5.05 (m, 1H), 4.20-4.09 (m, 1H), 3.68 (t, J = 10.4 Hz, 1H), 1.32 (d, J = 7.2 Hz, 3H), 1.30-1.24 (m, 6H) |
| | 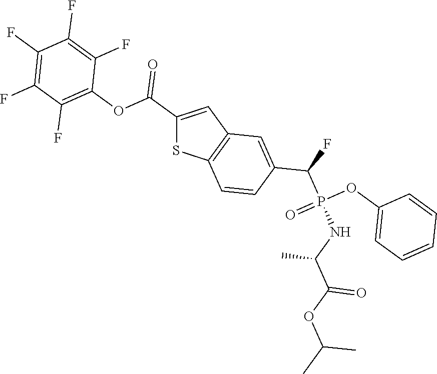 [SMILES: CC(C)OC(=O)[C@H](C)N[P@@](=O)(Oc1ccccc1)[C@H](F)c1ccc2sc(C(=O)Oc3c(F)c(F)c(F)c(F)c3F)cc2c1] | | |

The following intermediates in Table 69 were prepared using the synthetic procedure described above for allyl 5-((1S)-fluoro((((S)-1-oxo-1-propoxypropan-2-yl)amino)(phenoxy)phosphoryl)methyl)benzo[b]thiophene-2-carboxylate and utilizing the appropriate starting materials and modifications.

TABLE 69

| Name | Structure | LCMS |
|---|---|---|
| allyl 5-((1R)-fluoro((((S)-1- oxo-1-propoxypropan-2- yl)amino)(phenoxy)phosphoryl) methyl)benzo[b]thiophene-2- carboxylate | 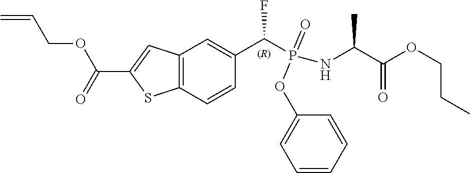 | 520.1 [M + H]⁺ |

The following phosphorus chiral intermediates in Table 70 were obtained by chiral SFC separation of the corresponding P-isomer mixtures, which were prepared using the synthetic procedure described above for allyl 5-((S) fluoro((((S)-2-oxo-1-propoxypropan-2-yl)amino)(phenoxy)phosphoryl)methyl)benzo[b]thiophene-2-carboxylate and utilizing the appropriate starting materials and modifications. Absolute stereochemical configuration at phosphorus is arbitrarily assigned as drawn.

TABLE 70

| | | | Preparative SFC |
|---|---|---|---|
| Name | Structure | LCMS | conditions for separation |
| allyl 5-((S)-fluoro((R)- (((S)-1-oxo-1- propoxypropan-2- yl)amino)(phenoxy) phosphoryl)methyl) benzo[b]thiophene-2- carboxylate | 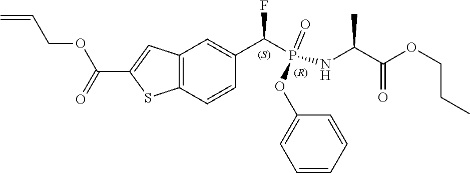 | 520.2 [M + H]⁺ | ChiralPak R,R-WHELK 250 × 21.2 mm 5 μm column; Mobile phase: A for CO₂ and B for MeOH + 0.1% NH₃H₂O; Gradient: B 50%; Flow rate: 40 mL/min; Back pressure: 100 bar; Column temperature: 35° C.; Wavelength: 220 nm; Cycle-time: 11 min |
| | (Peak 1, RT = 4.46 min) | | |
| allyl 5-((S)-fluoro((S)- (((S)-1-oxo-1- propoxypropan-2- yl)amino)(phenoxy) phosphoryl)methyl) benzo[b]thiophene-2- carboxylate | 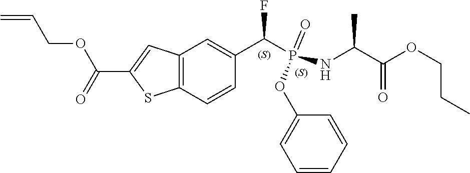 | 520.2 [M + H]⁺ | |
| | (Peak 2, RT = 6.54 min) | | |
| allyl 5-((R)- fluoro((R)-(((S)-1- oxo-1-propoxypropan- 2-yl)amino)(phenoxy) phosphoryl)methyl) benzo[b]thiophene-2- carboxylate | 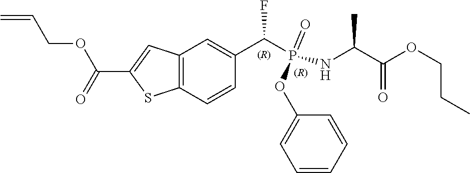 | 520.2 [M + H]⁺ | ChiralPak IH 250 × 21.2 mm 5 μm column; Mobile phase; A for CO₂ and B for MeOH + 0.1% NH₃H₂O; Gradient: B 35%; Flow rate: 40 mL/min; Back pressure: 100 bar; Column temperature: 35° C.; Wavelength: 220 nm; Cycle-time: 5 min |
| | (Peak 1, RT = 2.72 min) | | |
| allyl 5-((R)-fluoro((S)- (((S)-1-oxo-1- propoxypropan-2- yl)amino)(phenoxy) phosphoryl)methyl) benzo[b]thiophene-2- carboxylate | 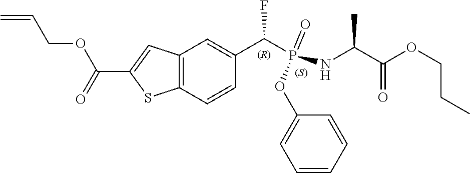 | 520.2 [M + H]⁺ | |
| | (Peak 2, RT = 4.11 min) | | |

The following intermediates in Table 71 were prepared using the synthetic procedure described above for 5-((1 S) fluoro((((S)-1-oxo-1-propoxypropan-2-yl)amino)(phenoxy)phosphoryl)methyl)benzo[b]thiophene-2-carboxylic acid and utilizing the appropriate starting materials and modifications.

TABLE 71

| Name | Structure | LCMS |
|---|---|---|
| 5-((S)-fluoro((R)-(((S)-1-oxo-1- propoxypropan-2- yl)amino)(phenoxy)phosphoryl) methyl)benzo[b]thiophene-2- carboxylic acid | 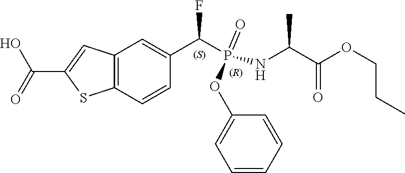 | 480.1 [M + H]⁺ |
| 5-((S)-fluoro((S)-(((S)-1-oxo-1- propoxypropan-2- yl)amino)(phenoxy)phosphoryl) methyl)benzo[b]thiophene-2- carboxylic acid | 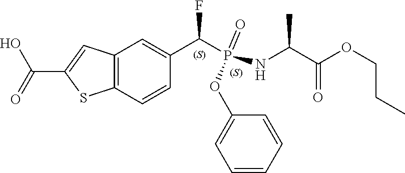 | 480.1 [M + H]⁺ |
| 5-((R)-fluoro((R)-(((S)-1-oxo- 1-propoxypropan-2- yl)amino)(phenoxy)phosphoryl) methyl)benzo[b]thiophene-2- carboxylic acid | 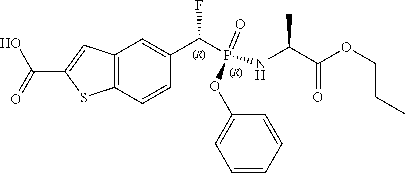 | 480.1 [M + H]⁺ |
| 5-((R)-fluoro((S)-(((S)-1-oxo-1- propoxypropan-2- yl)amino)(phenoxy)phosphoryl) methyl)benzo[b]thiophene-2- carboxylic acid | 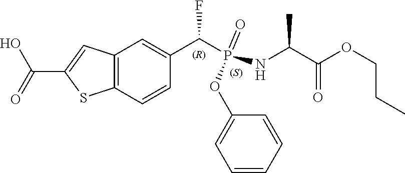 | 480.1 [M + H]⁺ |

### Synthesis of perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate and perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate

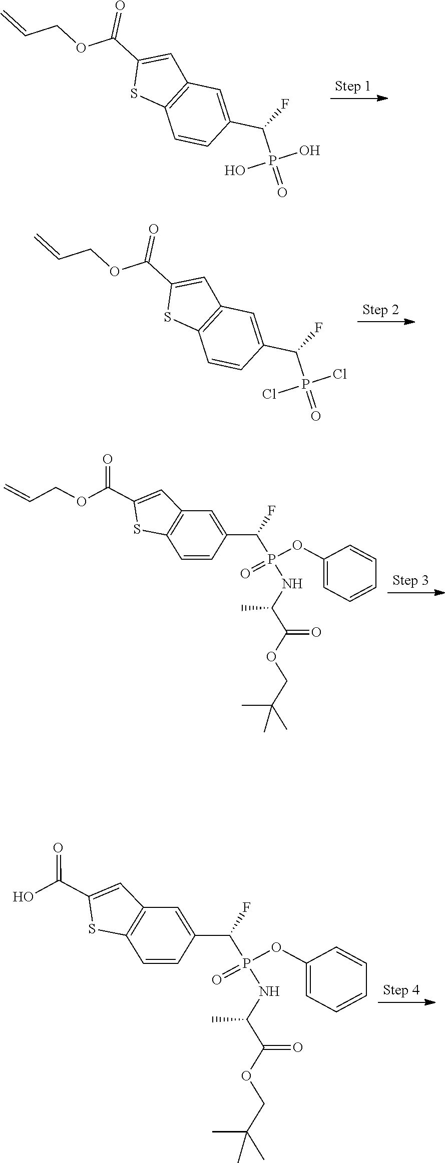 [SMILES: C=CCOC(=O)c1cc2cc([C@H](F)P(=O)(O)O)ccc2s1 ; C=CCOC(=O)c1cc2cc([C@H](F)P(=O)(Cl)Cl)ccc2s1 ; C=CCOC(=O)c1cc2cc([C@H](F)P(=O)(N[C@@H](C)C(=O)OCC(C)(C)C)Oc3ccccc3)ccc2s1 ; C[C@H](NP(=O)(Oc1ccccc1)[C@@H](F)c1ccc2sc(C(=O)O)cc2c1)C(=O)OCC(C)(C)C]

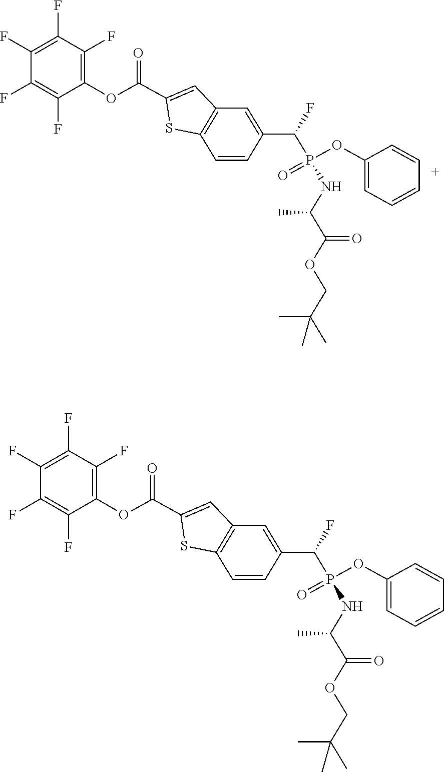 [SMILES: C[C@H](N[P@@](=O)(Oc1ccccc1)[C@@H](F)c1ccc2sc(C(=O)Oc3c(F)c(F)c(F)c(F)c3F)cc2c1)C(=O)OCC(C)(C)C ; C[C@H](N[P@](=O)(Oc1ccccc1)[C@@H](F)c1ccc2sc(C(=O)Oc3c(F)c(F)c(F)c(F)c3F)cc2c1)C(=O)OCC(C)(C)C]

### Step 1: Preparation of allyl (R)-5-((dichlorophosphoryl)fluoromethyl)benzo[b]thiophene-2-carboxylate

To a solution of (R)-((2-((allyloxy)carbonyl)benzo[b]thiophen-5-yl)fluoromethyl)phosphonic acid (2.0 g, 6.1 mmol, 1.0 eq) in DCM (30 mL) was added 3 drops of DMF (0.03 mL) cooled to 0° C., then the reaction was added oxalyl chloride (2.3 g, 18.1 mmol, 3 eq) slowly, after that the reaction was warmed to 45° C. and stirred 20 min to give a yellow clean solution. The reaction mixture was concentrated under reduced pressure to give allyl (R)-5-((dichlorophosphoryl)fluoromethyl)benzo[b]thiophene-2-carboxylate (2.0 g, crude) as a yellow oil and was used into the next step without further purification. LCMS (ESI) m/z=358.9 [M+H]⁺.

### Step 2: Preparation of Allyl 5-((1R)-fluoro((((S)-1-(neopentyloxy)-1-oxopropan-2-yl)amino)(phenoxy)phosphoryl)methyl)benzo[b]thiophene-2-carboxylate

To a solution of allyl (R)-5-((dichlorophosphoryl)fluoromethyl)benzo[b]thiophene-2-carboxylate (2.0 g, 5.4 mmol, 1.0 eq) in DCM (40 mL) was cooled to 0° C. and was added a solution of phenol (0.5 g, 5.4 mmol, 1.0 eq) in DCM (10 mL), then the mixture was added N,N-diisopropylethylamine (2.1 g, 16 mmol, 3.0 eq) in DCM (40 mL) dropwise. The reaction was stirred at 0° C. for 10 min and was added 2,2-dimethylpropyl (2S)-2-aminopropanoate (0.9 g, 5.4 mmol, 1.0 eq) in DCM (20 mL). Then the reaction was warmed to 25° C. and stirred for 1 h to give a light-yellow clean solution. The reaction mixture was concentrated under reduced pressure to give a residue. The residue was purified by flash column chromatography on silica gel to get allyl 5-((1R)-fluoro((((S)-1-(neopentyloxy)-1-oxopropan-2-yl)amino)(phenoxy)phosphoryl)methyl)benzo[b]thiophene-2-carboxylate (1.7 g, 3.1 mmol, 57% yield) was obtained as a yellow oil. LCMS (ESI) m/z=548.2. ¹H NMR (400 MHz, CDCl₃) δ 8.10 (s, 1H), 8.07-7.99 (m, 1H), 7.91 (d, J=8.4 Hz, 1H), 7.68-7.59 (m, 1H), 7.41-7.25 (m, 3H), 7.23-7.06 (m, 3H), 6.12-5.90 (m, 2H), 5.48-5.44 (m, 1H), 5.39-5.30 (m, 1H), 4.87 (d, J=5.6 Hz, 2H), 4.22-4.07 (m, 2H), 3.90-3.83 (m, 1H), 3.74-3.63 (m, 2H), 1.38-1.30 (m, 3H), 0.92 (s, 9H).

### Step 3: Preparation of 5-((1R)-fluoro((((S)-1-(neopentyloxy)-1-oxopropan-2-yl)amino)(phenoxy)phosphoryl)methyl)benzo[b]thiophene-2-carboxylic acid

To a solution of allyl 5-((1R)-fluoro((((S)-1-(neopentyloxy)-1-oxopropan-2-yl)amino)(phenoxy)phosphoryl)methyl)benzo[b]thiophene-2-carboxylate (1.7 g, 3.1 mmol, 1.0 eq) in DCM (20 mL) was added pyrrolidine (0.4 g, 6.2 mmol, 2.0 eq) and Pd(PPh₃)₄(0.4 g, 0.3 mmol, 0.1 eq). Then the reaction stirred for 15 min to give a yellow clean solution. The reaction mixture was concentrated under reduced pressure to give a residue. The residue was purified by reversed phase (TFA) to lyophilized to give 5-((1R)-fluoro((((S)-1-(neopentyloxy)-1-oxopropan-2-yl)amino)(phenoxy)phosphoryl)methyl)benzo[b]thiophene-2-carboxylic acid (1.3 g, 2.6 mmol, 83% yield) as a white solid. LCMS (ESI) m/z=508.2

### Step 4: Preparation of Perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate and perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate

To a solution of 5-((1R)-fluoro((((S)-1-(neopentyloxy)-1-oxopropan-2-yl)amino)(phenoxy)phosphoryl)methyl)benzo[b]thiophene-2-carboxylic acid (1.2 g, 2.4 mmol, 1.0 eq) in pyridine (100 mL) was added 2,3,4,5,6-pentafluorophenyl 2,2,2-trifluoroacetate (0.66 g, 2.4 mmol, 1.0 eq) at 0° C., the mixture was stirred at 25° C. for 10 min to give a yellow clean solution. The reaction mixture was concentrated under reduced pressure to give a residue. The residue was purified by flash column chromatography on silica gel to give perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate or perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate (0.4 g, 0.59 mmol, 25% yield, Rt=1.883 min, Peak 2) as a yellow oil and perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate or perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate (0.50 g, 0.74 mmol, 32% yield, Rt=1.442 min, Peak 1) as a yellow oil.

Peak 2: perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate: LCMS (ESI) m/z=674.0 [M+H]⁺. ¹H NMR (400 MHz, CDCl₃) δ 8.34 (s, 1H), 8.09 (s, 1H), 7.97 (d, J=8.4 Hz, 1H), 7.68 (d, J=8.4 Hz, 1H), 7.40-7.35 (m, 2H), 7.32-7.27 (m, 2H), 7.24-7.19 (m, 1H), 6.07-5.89 (m, 1H), 4.16-4.05 (m, 1H), 3.88-3.76 (m, 2H), 3.72-3.64 (m, 1H), 1.15 (d, J=7.2 Hz, 3H), 0.91 (s, 9H)

Peak 1: perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate or perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate: LCMS (ESI) m/z=674.0 [M+H]⁺. ¹H NMR (400 MHz, CDCl₃) δ 8.34 (s, 1H), 8.12 (s, 1H), 7.96 (d, J=8.4 Hz, 1H), 7.74 (d, J=8.4 Hz, 1H), 7.29 (s, 1H), 7.25 (s, 1H), 7.16-7.05 (m, 3H), 6.13-5.96 (m, 1H), 4.22-4.10 (m, 1H), 3.89-3.83 (m, 1H), 3.73-3.66 (m, 1H), 3.62-3.54 (m, 1H), 1.33 (d, J=7.2 Hz, 3H), 0.92 (s, 9H).

### Synthesis of 7-(fluoro((((S)-1-oxo-1-propoxypropan-2-yl)amino)(phenoxy)phosphoryl)methyl)-2-naphthoic acid

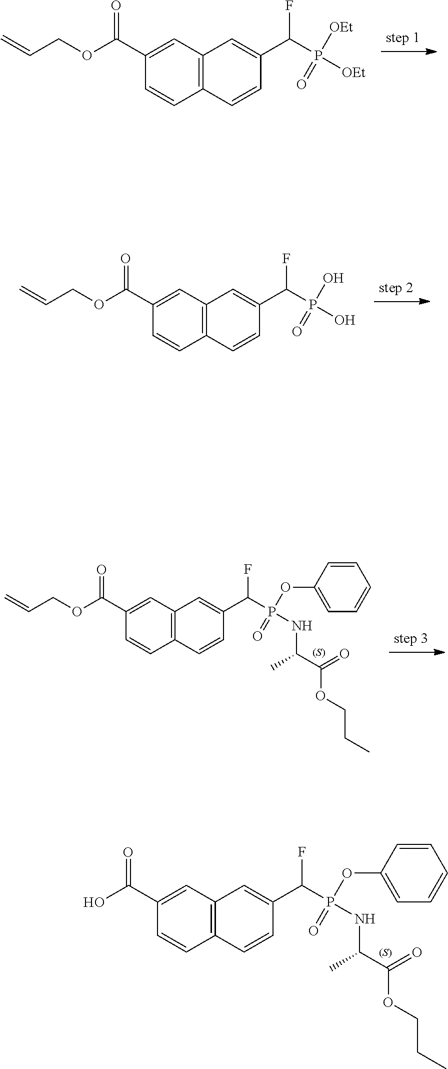

### Step 1: Preparation of ((7-((allyloxy)carbonyl)naphthalen-2-yl)fluoromethyl)phosphonic acid

To a solution of allyl 7-((diethoxyphosphoryl)fluoromethyl)-2-naphthoate (1 g, 2.63 mmol, 1.0 eq.) in DCM (10 mL) were added TMSBr (4.0 g, 26.3 mmol, 10.0 eq.). The mixture was stirred at room temperature for 16 h. After completion, the reaction mixture was concentrated under reduced pressure. The residue was purified by flash column chromatography on silica gel to afford ((7-((allyloxy)carbonyl)naphthalen-2-yl)fluoromethyl)phosphonic acid (680 mg, 2.10 mmol, 80%) as a colorless oil. LCMS (ESI): m/z=325 [M+H]⁺.

### Step 2: Preparation of Allyl 7-(fluoro((((S)-1-oxo-1-propoxypropan-2-yl)amino)(phenoxy)phosphoryl)methyl)-2-naphthoate

Oxalyl chloride (1.32 g, 10.5 mmol, 5 eq.) was added dropwise to the solution of afford ((7-((allyloxy)carbonyl)naphthalen-2-yl)fluoromethyl)phosphonic acid (680 mg, 2.10 mmol, 1 eq.) in dry DCM (20 mL) and DMF (0.1 mL) at 25° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (20 mL), then added to a mixture of phenol (197 mg, 2.10 mmol, 1.0 eq.) and TEA (2.12 g, 21.0 mmol, 10 eq.) in anhydrous DCM (20 mL) at −20° C. The reaction was stirred at −20° C. for an additional 1 hr. Then, propyl L-alaninate (550 mg, 4.2 mmol, 2.0 eq.) was added into the above mixture at 0° C. The reaction was stirred at 25° C. for an additional 1 hr. After completion, the reaction was poured into ice-water (10 mL) and extracted with DCM (10 mL×2). The organic layers were combined and washed with brine (10 mL), dried over anhydrous Na₂SO₄, and concentrated under reduced pressure. The residue was purified by Biotage® C18 column chromatography to afford allyl 7-(fluoro((((S)-1-oxo-1-propoxypropan-2-yl)amino)(phenoxy)phosphoryl)methyl)-2-naphthoate (323 mg, 0.63 mmol, 30%) as a brown solid. LCMS (ESI): m/z=514 [M+H]⁺.

### Step 3: Preparation of 7-(fluoro((((S)-1-oxo-1-propoxypropan-2-yl)amino)(phenoxy)phosphoryl)methyl)-2-naphthoic acid

To a solution of allyl 7-(fluoro((((S)-1-oxo-1-propoxypropan-2-yl)amino)(phenoxy)phosphoryl)methyl)-2-naphthoate (323 mg, 0.63 mmol, 1.0 eq) in DCM (10 mL) were added Pd(PPh₃)₄(69 mg, 0.06 mmol, 0.1 eq.) and pyrrolidine (45 mg, 0.63 mmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 7-(fluoro((((S)-1-oxo-1-propoxypropan-2-yl)amino)(phenoxy)phosphoryl)methyl)-2-naphthoic acid (228 mg, 0.48 mmol, 76%) as a yellow solid. LCMS (ESI): m/z=474 [M+H]⁺.

### Synthesis of ((2-((4-nitrophenoxy)carbonyl)-1H-indol-5-yl)methyl)phosphonic acid

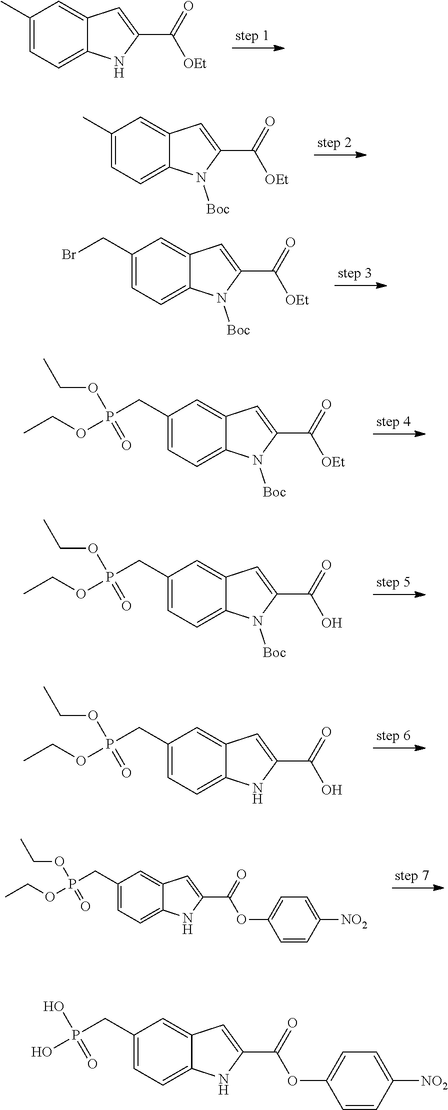

### Step 1: Preparation of 1-(tert-butyl) 2-ethyl 5-methyl-1H-indole-1,2-dicarboxylate

To a solution of 1-tert-butyl 2-ethyl 5-[(diethoxyphosphoryl)methyl]-1H-indole-1,2-dicarboxylate (10 g, 5.2 mmol) in THF (100 mL) was added NaH (0.12 g, 4.9 mmol) at 0° C. and stirred for 0.5 h, then di-tert-butyl dicarbonate (13.0 g, 59.0 mmol) was added in it and stirred at 25° C. for 12 h to give a white suspension. The reaction mixture was quenched by water (100 mL) at 0° C., then extracted with EtOAc (150 mL×2), the combined organic layers were washed with saturated brine (200 mL×2), dried over Na₂SO₄, filtered and concentrated under reduced pressure to give a residue. The residue was purified by column chromatography to give 1-(tert-butyl) 2-ethyl 5-methyl-1H-indole-1,2-dicarboxylate (15 g, 49 mmol, 90% yield) as a yellow oil. LCMS (ESI) m/z=304.5 [M+H]⁺.

### Step 2: Preparation of 1-(tert-butyl) 2-ethyl 5-(bromomethyl)-1H-indole-1,2-dicarboxylate

To a solution of 1-(tert-butyl) 2-ethyl 5-methyl-1H-indole-1,2-dicarboxylate (15 g, 49 mmol, 1.0 eq) in chloroform (120 mL) was added BPO (477 mg, 2.0 mmol, 0.04 eq) and N-bromosuccinimide (8.8 g, 49 mmol, 1.0 eq), the mixture was stirred at 120° C. for 12 h to give a yellow solution. The reaction mixture was concentrated under reduced pressure to give a residue. The residue was purified by column chromatography to give 1-(tert-butyl) 2-ethyl 5-(bromomethyl)-1H-indole-1,2-dicarboxylate (15 g, 39 mmol, 79% yield) as a yellow oil. LCMS (ESI) m/z=281.9 [M+H]⁺.

### Step 3: Preparation of 1-(tert-butyl) 2-ethyl 5-((diethoxyphosphoryl)methyl)-1H-indole-1,2-dicarboxylate

A solution of 1-(tert-butyl) 2-ethyl 5-(bromomethyl)-1H-indole-1,2-dicarboxylate (3.2 g, 8.4 mmol) in triethyl phosphite (14 g, 84 mmol) was stirred at 100° C. for 12 h to give a colorless solution. The reaction mixture was diluted with water (100 mL) and extracted with EtOAc (100 mL×2), the combined organic layers were washed with saturated brine (150 mL×2), dried over Na₂SO₄, filtered and concentrated under reduced pressure to give a residue. The residue was purified by column chromatography to give 1-(tert-butyl) 2-ethyl 5-((diethoxyphosphoryl)methyl)-1H-indole-1,2-dicarboxylate (2.3 g, 5.2 mmol, 62% yield) as a colorless oil. ¹H NMR (400 MHz, CDCl₃) δ 8.00 (d, J=8.8 Hz, 1H), 7.52 (s, 1H), 7.39-7.33 (m, 1H), 7.04 (s, 1H), 4.45-4.44 (m, 1H), 4.37 (q, J=7.2 Hz, 1H), 4.03-3.90 (m, 4H), 3.28-3.16 (m, 2H), 1.62 (s, 9H), 1.27-1.21 (m, 6H), 1.16-1.12 (m, 3H).

### Step 4: Preparation of 1-(tert-butoxycarbonyl)-5-((diethoxyphosphoryl)methyl)-1H-indole-2-carboxylic acid

To a solution of 1-tert-butyl 2-ethyl 5-[(diethoxyphosphoryl)methyl]-1H-indole-1,2-dicarboxylate (4.6 g, 5.2 mmol) in MeOH (15 mL), THF (15 mL) and H₂O (15 mL) was added LiOH—H₂O (1.8 g, 42 mmol) and stirred at 25° C. for 1 h to give a white suspension. The reaction mixture was used saturated 2 M HCl to adjust the pH=4-5, the reaction mixture was concentrated under reduced pressure to give 1-(tert-butoxycarbonyl)-5-((diethoxyphosphoryl)methyl)-1H-indole-2-carboxylic acid (7.0 g, crude) as a yellow oil. LCMS (ESI) m/z=412.2 [M+H]⁺.

### Step 5: Preparation of 5-((diethoxyphosphoryl)methyl)-1H-indole-2-carboxylic acid

To a solution of 1-(tert-butoxycarbonyl)-5-((diethoxyphosphoryl)methyl)-1H-indole-2-carboxylic acid (7.0 g, 17 mmol) in DCM (30 mL) was added TFA (30 mL) and stirred at 25° C. for 1 h to give a brown solution. The reaction mixture was concentrated under reduced pressure to give 5-((diethoxyphosphoryl)methyl)-1H-indole-2-carboxylic acid (7.0 g, crude) as a brown oil. LCMS (ESI) m/z=311.9 [M+H]⁺.

### Step 6: Preparation of 4-nitrophenyl 5-((diethoxyphosphoryl)methyl)-1H-indole-2-carboxylate

To a solution of 5-((diethoxyphosphoryl)methyl)-1H-indole-2-carboxylic acid (1.2 g, 3.9 mmol) in EtOAc (70 mL) was added 4-nitrophenol (0.64 g, 4.6 mmol), dicyclohexylcarbodiimide (0.95 g, 4.6 mmol) and 4-dimethylaminopyridine (0.70 g, 5.8 mmol), then the mixture was stirred at 25° C. for 3 h to give a white suspension. The reaction mixture was filtered and the filter was concentrated to give a residue. The residue was purified by column chromatography to give 4-nitrophenyl 5-((diethoxyphosphoryl)methyl)-1H-indole-2-carboxylate (0.90 g, 2.1 mmol, 54% yield) as a yellow solid. LCMS (ESI) m/z=433.0 [M+H]⁺. ¹H NMR (400 MHz, CD₃OD) δ 8.35 (d, J=9.2 Hz, 2H), 7.65 (d, J=2.4 Hz, 1H), 7.58-7.53 (m, 2H), 7.46 (d, J=8.8 Hz, 1H), 7.40 (s, 1H), 7.30 (m, 1H), 4.07-3.99 (m, 4H), 1.25 (t, J=7.2 Hz, 6H).

### Step 7: Preparation of ((2-((4-nitrophenoxy)carbonyl)-1H-indol-5-yl)methyl)phosphonic acid

To a solution of 4-nitrophenyl 5-((diethoxyphosphoryl)methyl)-1H-indole-2-carboxylate (25 mg, 58 μmol) and BSTFA (59 mg, 0.23 mmol) in DCM (0.5 mL) was dropwise added a solution of trimethylsilyl iodide (69 mg, 0.35 mmol) in DCM (0.5 mL) at 0° C., then the mixture solution was stirred at 0° C. for 1 h to give a brown solution. The reaction mixture was concentrated under reduced pressure to give ((2-((4-nitrophenoxy)carbonyl)-1H-indol-5-yl)methyl)phosphonic acid (25 mg, crude) as a brown solid. LCMS (ESI) m/z=377.0 [M+H]⁺.

### Synthesis of 5-(difluoro((((S)-1-isopropoxy-1-oxopropan-2-yl)amino)(phenoxy)phosphoryl)methyl)benzo[b]thiophene-2-carboxylic acid

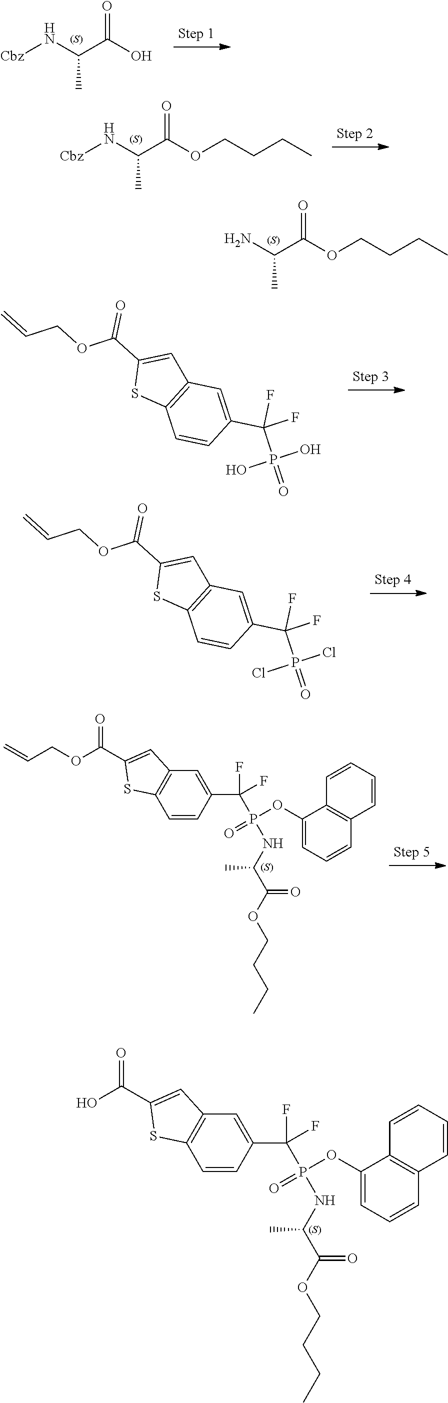

### Step 1: Preparation of Allyl 5-((diethoxyphosphoryl)difluoromethyl)benzo[b]thiophene-2-carboxylate

To a solution of 5-((diethoxyphosphoryl)difluoromethyl)benzo[b]thiophene-2-carboxylic acid (5 g, 14 mmol, 1 eq) in dimethyl sulfoxide (40 mL) was added disodium carbonate (4.3 g, 41 mmol, 3 eq) and 3-bromoprop-1-ene (1.7 g, 14 mmol, 1 eq), the mixture was stirred at 25° C. for 12 h to give a white suspension. The mixture was diluted with water (200 mL) and extracted with EtOAc (200 mL×2), the combined organic layers were washed with saturated brine (200 mL×2), dried over Na₂SO₄, filtered and concentrated under reduced pressure to give a residue. The residue was purified by column chromatography to give allyl 5-((diethoxyphosphoryl)difluoromethyl)benzo[b]thiophene-2-carboxylate (4.6 g, 11.3 mmol, 83% yield) as a white oil. ¹H NMR (400 MHz, CDCl₃) δ 8.17-8.13 (m, 2H), 7.96 (d, J=8.8 Hz, 1H), 7.70 (d, J=8.4 Hz, 1H), 6.13-5.99 (m, 1H), 5.50-5.42 (m, 1H), 5.37-5.32 (m, 1H), 4.87 (d, J=5.6 Hz, 2H), 4.27-4.12 (m, 4H), 1.33 (t, J=7.2 Hz, 6H).

### Step 2: Preparation of ((2-((allyloxy)carbonyl)benzo[b]thiophen-5-yl)difluoromethyl)phosphonic acid

To a solution of allyl 5-((diethoxyphosphoryl)difluoromethyl)benzo[b]thiophene-2-carboxylate (4.6 g, 11.3 mmol, 1 eq) in DCM (40 mL) was added iodotrimethylsilane (9.0 g, 45 mmol, 4 eq) at 0° C., the mixture was stirred at 0° C. for 2 h. The reaction mixture was concentrated under reduced pressure to give a residue. The residue was purified by reversed phase (TFA) to give ((2-((allyloxy) carbonyl)benzo[b]thiophen-5-yl)difluoromethyl)phosphonic acid (2.3 g, 6.6 mmol, 58% yield) as a white solid. LCMS (ESI) m/z=348.7

### Step 3: Preparation of Allyl 5-((dichlorophosphoryl)difluoromethyl)benzo[b]thiophene-2-carboxylate

To a solution of ((2-((allyloxy) carbonyl)benzo[b]thiophen-5-yl)difluoromethyl)phosphonic acid (1 g, 2.9 mmol, 1 eq) in DCM (3 mL) was added DMF (21 mg, 0.29 mmol, 0.1 eq), oxalic dichloride (1.2 g, 28 mmol, 3 eq) was dropwise at 0° C., the mixture was stirred at 40° C. for 1 h to give a yellow solution. The reaction mixture was concentrated under reduced pressure to give allyl 5-((dichlorophosphoryl)difluoromethyl)benzo[b]thiophene-2-carboxylate (1 g, crude) as a yellow solid. LCMS (ESI) m/z=376.6.

### Step 4: Preparation of Allyl 5-(((((S)-1-butoxy-1-oxopropan-2-yl)amino)(naphthalen-1-yloxy)phosphoryl)difluoromethyl)benzo[b]thiophene-2-carboxylate

To a solution of prop-2-en-1-yl 5-[(dichlorophosphoryl)difluoromethyl]-1-benzothiophene-2-carboxylate (0.2 g, 0.52 mmol, 1 eq) in methylene chloride (10 mL) was added naphthalen-1-ol (60 mg, 0.42 mmol, 0.8 eq), the mixture was stirred at 0° C. for 5 min, then a solution of N,N-diisopropylethylamine (0.2 g, 1.6 mmol, 3.0 eq) in methylene chloride (10 mL) was added to the mixture, then a solution of butyl (2S)-2-aminopropanoate (75 mg, 0.52 mmol, 1 eq) in methylene chloride (10 mL) was added to the mixture, the mixture was stirred at 0° C. for 30 min to give yellow solution. The reaction mixture was concentrated under reduced pressure to give a residue.

The residue was purified by column chromatography to give prop-2-en-1-yl 5-[({[(2S)-1-butoxy-1-oxopropan-2-yl]amino}(naphthalen-1-yloxy)phosphoryl)difluoromethyl]-1-benzothiophene-2-carboxylate (0.15 g, 0.24 mmol, 46.4% yield) as a yellow oil. LCMS (ESI) m/z=602.1

### Step 5: Preparation of 5-(((((S)-1-butoxy-1-oxopropan-2-yl)amino)(naphthalen-1-yloxy)phosphoryl)difluoromethyl)benzo[b]thiophene-2-carboxylic acid

To a solution of prop-2-en-1-yl 5-[({[(2S)-1-butoxy-1-oxopropan-2-yl]amino}(naphthalen-1-yloxy)phosphoryl)difluoromethyl]-1-benzothiophene-2-carboxylate (0.14 g, 0.23 mmol, 1 eq) in methylene chloride (1.0 mL) was added Pd(PPh₃)₄(27 mg, 23 μmol, 0.1 eq), the mixture was stirred at 0° C. for 5 min, then pyrrolidine (17 mg, 0.23 mmol, 1 eq) was added to the mixture, the mixture was stirred at 25° C. for 5 min to give yellow solution. The reaction mixture was concentrated under reduced pressure to give a residue. The residue was purified by reversed phase (TFA) to give 5-[({[(2S)-1-butoxy-1-oxopropan-2-yl]amino}(naphthalen-1-yloxy)phosphoryl)difluoromethyl]-1-benzothiophene-2-carboxylic acid (97 mg, 0.17 mmol, 74.6% yield) as a yellow oil. LCMS (ESI) m/z=562.1

### Synthesis of 5-(((((S)-1-butoxy-1-oxopropan-2-yl)(methyl)amino)(phenoxy)phosphoryl)methyl)benzo[b]thiophene-2-carboxylic acid

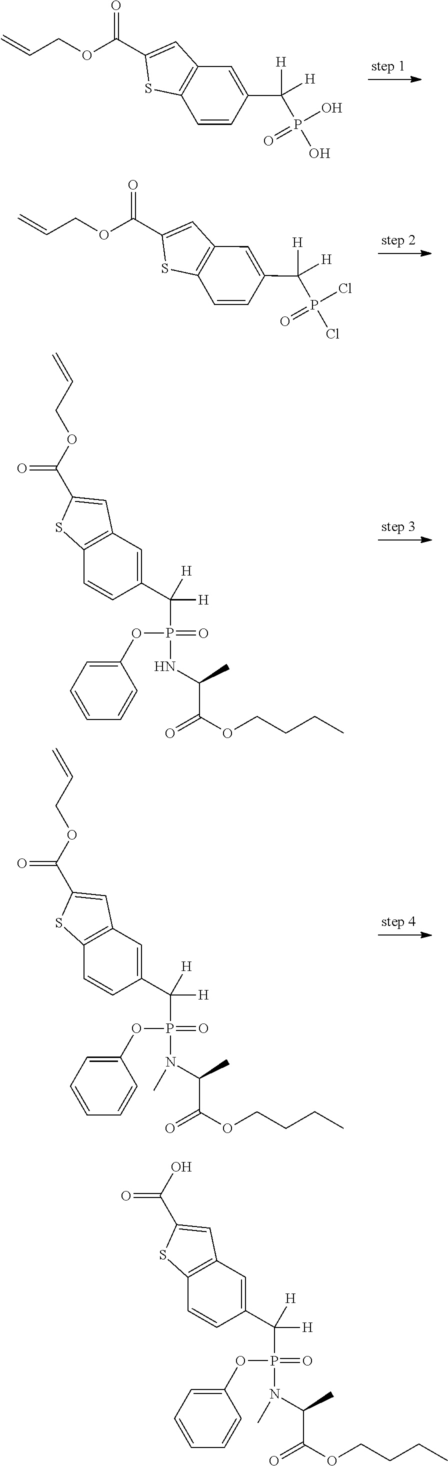

### Step 1: Preparation of Allyl 5-((dichlorophosphoryl)methyl)benzo[b]thiophene-2-carboxylate

To a solution of ((2-((allyloxy)carbonyl)benzo[b]thiophen-5-yl)methyl)phosphonic acid (0.1 g, 0.32 mmol, 1 eq) in DCM (2 mL) was added DMF (2.3 mg, 32 μmol, 0.1 eq). The mixture was stirred at 0° C. for 5 min, then a solution of oxalyl chloride (0.12 g, 0.96 mmol, 3 eq) in DCM (2 mL) was added to the mixture, the mixture was stirred at 40° C. for 1 h to give yellow solution. The reaction mixture was concentrated under reduced pressure to give allyl 5-((dichlorophosphoryl)methyl)benzo[b]thiophene-2-carboxylate (0.100 g, crude) as a yellow oil. LCMS (ESI) m/z=341.0 (MeOH quench)

### Step 2: Preparation of Allyl 5-(((((S)-1-butoxy-1-oxopropan-2-yl)amino)(phenoxy)phosphoryl)methyl)benzo[b]thiophene-2-carboxylate

To a solution of allyl 5-((dichlorophosphoryl)methyl)benzo[b]thiophene-2-carboxylate (0.10 g, 0.29 mmol, 1 eq) in DCM (2 mL) was added phenol (22 mg, 0.23 mmol, 0.8 eq) at 0° C. for 30 min, then a solution of DIEA (0.1 g, 0.86 mmol, 3.0 eq) in DCM (2 mL) was added to the mixture at 0° C. for 15 min, then a solution of butyl L-alaninate (42 mg, 0.29 mmol, 1 eq) in DCM (2 mL) was added to the mixture at 0° C. for 15 min to give yellow solution. The reaction mixture was concentrated under reduced pressure to give a residue. The residue was purified by column chromatography to give allyl 5-(((((S)-1-butoxy-1-oxopropan-2-yl)amino)(phenoxy)phosphoryl)methyl)benzo[b]thiophene-2-carboxylate (80 mg, 0.15 mmol, 80% yield,) as a yellow oil. LCMS (ESI) m/z=516.2 [M+H]⁺.

### Step 3: Preparation of Allyl 5-(((((S)-1-butoxy-1-oxopropan-2-yl)(methyl)amino)(phenoxy)phosphoryl)methyl)benzo[b]thiophene-2-carboxylate

To a solution of allyl 5-(((((S)-1-butoxy-1-oxopropan-2-yl)amino)(phenoxy)phosphoryl)methyl)benzo[b]thiophene-2-carboxylate (0.2 g, 0.39 mmol, 1 eq) in THF (5 mL) was added LDA (83 mg, 0.77 mmol, 2 eq) and methyl iodide (0.55 g, 3.9 mmol, 10 eq), the mixture was stirred at −70° C. for 15 min to give yellow solution. The reaction mixture was added water (0.5 mL) and concentrated under reduced pressure to give a residue. The residue was purified by column chromatography to give allyl 5-(((((S)-1-butoxy-1-oxopropan-2-yl)(methyl)amino)(phenoxy)phosphoryl)methyl)benzo[b]thiophene-2-carboxylate (60 mg, 0.11 mmol, 28% yield) as a yellow oil. LCMS (ESI) m/z=530.2 [M+H]⁺.

### Step 4: Preparation of 5-(((((S)-1-butoxy-1-oxopropan-2-yl)(methyl)amino)(phenoxy)phosphoryl)methyl)benzo[b]thiophene-2-carboxylic acid

To a solution of allyl 5-(((((S)-1-butoxy-1-oxopropan-2-yl)(methyl)amino)(phenoxy)phosphoryl)methyl)benzo[b]thiophene-2-carboxylate (60 mg, 0.11 mmol, 1 eq) in DCM (2 mL) was added pyrrolidine (6.4 mg, 90 μmol, 0.8 eq) and Pd(PPh₃)₄(1.3 mg, 1.1 μmol, 0.01 eq), the mixture was stirred at 25° C. for 5 min to give yellow solution. The reaction mixture was added water and concentrated under reduced pressure to give a residue. The residue was purified by reversed phase (TFA) then lyophilized to give 5-(((((S)-1-butoxy-1-oxopropan-2-yl)(methyl)amino)(phenoxy)phosphoryl)methyl)benzo[b]thiophene-2-carboxylic acid (49 mg, 99 μmol, 88% yield) as a yellow oil. LCMS (ESI) m/z=490.1 [M+H]⁺.

### Synthesis of perfluorophenyl 5-(fluoro((((S)-1-oxo-1-propoxypropan-2-yl)amino)(phenoxy)phosphoryl)methyl)benzo[b]thiophene-2-carboxylate

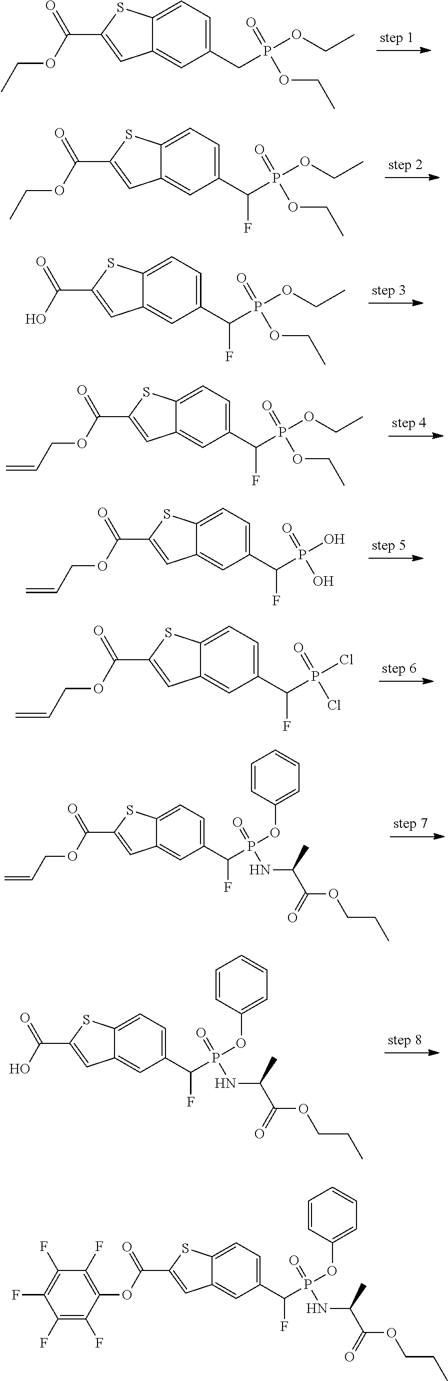

### Step 1: Preparation of Ethyl 5-((diethoxyphosphoryl)fluoromethyl)benzo[b]thiophene-2-carboxylate

To a solution of ethyl 5-((diethoxyphosphoryl)methyl)benzo[b]thiophene-2-carboxylate (58 g, 0.16 mol, 1 eq) in THF (600 mL) was added lithium bis(trimethylsilyl)azanide (170 mL, 0.17 mol, 1.1 eq, 1.0 M in THF) at −70° C., then N-(benzenesulfonyl)-N-fluorobenzenesulfonamide (54 g, 0.17 mol, 1.1 eq) was added at −70° C., then stirred at −70° C. for 0.5 h. The reaction mixture was quenched by water (500 mL) at −70° C., then extracted with EtOAc (500 mL×2), the combined organic layers were washed with saturated brine (400 mL×2), dried over Na₂SO₄, filtered and concentrated under reduced pressure to give a residue. The residue was purified by flash silica gel chromatography to give ethyl 5-((diethoxyphosphoryl)fluoromethyl)benzo[b]thiophene-2-carboxylate (26 g, 67 mmol, 43% yield) as a yellow oil. ¹H NMR (400 MHz, CDCl3) δ 8.00 (s, 1H), 7.92 (s, 1H), 7.83 (d, J=8.4 Hz, 1H), 7.51 (d, J=8.4 Hz, 1H), 5.84-5.63 (m, 1H), 4.39-4.29 (m, 2H), 4.09-3.96 (m, 4H), 1.35 (t, J=7.1 Hz, 3H), 1.23-1.19 (m, 6H).

### Step 2: Preparation of 5-((diethoxyphosphoryl)fluoromethyl)benzo[b]thiophene-2-carboxylic acid

To a solution of ethyl 5-((diethoxyphosphoryl)fluoromethyl)benzo[b]thiophene-2-carboxylate (15 g, 40 mmol, 1 eq) in THF (40 mL) was dropwise added LiOH—H₂O (1.8 g, 44 mmol, 1.1 eq) in H₂O (40 mL) at 0° C. and stirred at 25° C. for 1 h to give a yellow solution. The reaction mixture was extracted with EtOAc (80 mL), then the aqueous phase was used 1M HCl to adjust the pH=4˜3 extracted with EtOAc (100 mL×2), the combined organic layers were dried over Na₂SO₄, filtered and concentrated under reduced pressure to give 5-((diethoxyphosphoryl)fluoromethyl)benzo[b]thiophene-2-carboxylic acid (11 g, crude) as a white solid. LCMS (ESI) m/z=346.8 [M+H]⁺.

### Step 3: Preparation of Allyl 5-((diethoxyphosphoryl)fluoromethyl)benzo[b]thiophene-2-carboxylate

To a solution of 5-((diethoxyphosphoryl)fluoromethyl)benzo[b]thiophene-2-carboxylic acid (11 g, 32 mmol, 1 eq) in DMF (120 mL) was added sodium carbonate (6.7 g, 63 mmol, 2 eq) and stirred at 25° C. for 30 min, then 3-bromoprop-1-ene (7.7 g, 63 mmol, 2 eq) was added in it and stirred at 25° C. for 5 h to give a white suspension. The reaction mixture was diluted with water (300 mL) and extracted with EtOAc (400 mL×3), the combined organic layers were washed with saturated brine (500 mL×3), dried over Na₂SO₄, filtered and concentrated under reduced pressure to give a residue. The residue was purified by column chromatography to give allyl 5-((diethoxyphosphoryl)fluoromethyl)benzo[b]thiophene-2-carboxylate (10 g, 26 mmol, 82% yield) as a white solid. ¹H NMR (400 MHz, CDCl₃) δ 8.10 (s, 1H), 8.05-7.98 (m, 1H), 7.91 (d, J=8.4 Hz, 1H), 7.59 (d, J=8.4 Hz, 1H), 6.13-5.97 (m, 1H), 5.91-5.71 (m, 1H), 5.52-5.40 (m, 1H), 5.33 (dd, J=1.2, 10.4 Hz, 1H), 4.86 (dd, J=1.2, 5.6 Hz, 2H), 4.19-4.03 (m, 4H), 1.33-1.24 (m, 6H).

### Step 4: Preparation of ((2-((allyloxy)carbonyl)benzo[b]thiophen-5-yl)fluoromethyl)phosphonic acid

To a solution of allyl 5-((diethoxyphosphoryl)fluoromethyl)benzo[b]thiophene-2-carboxylate (9 g, 23 mmol, 1 eq) and (E)-(trimethylsilyl 2,2,2-trifluoro-N-(trimethylsilyl)ethanimidate) (18 g, 70 mmol, 3 eq) in DCM (100 mL) was added iodotrimethylsilane (16 g, 93 mmol, 4 eq) at 0° C. and stirred at 0° C. for 1 h to give a brown solution. The reaction mixture was concentrated under reduced pressure to give a residue. The crude was purified by reverse phase chromatography (TFA) then lyophilization to give ((2-((allyloxy)carbonyl)benzo[b]thiophen-5-yl)fluoromethyl)phosphonic acid (6.0 g, 18 mmol, 78% yield) as a yellow solid. ¹H NMR (400 MHz, DMSO-d₆) δ 8.31 (s, 1H), 8.14-8.05 (m, 2H), 7.59 (d, J=8.4 Hz, 1H), 6.13-5.98 (m, 1H), 5.90-5.76 (m, 1H), 5.44 (dd, J=1.6, 17.2 Hz, 1H), 5.31 (dd, J=1.6, 10.4 Hz, 1H), 4.86-4.83 (m, 2H).

### Step 5: Preparation of Allyl 5-((dichlorophosphoryl)fluoromethyl)benzo[b]thiophene-2-carboxylate

To a solution of ((2-((allyloxy)carbonyl)benzo[b]thiophen-5-yl)fluoromethyl)phosphonic acid (2 g, 6.0 mmol, 1 eq) and DMF (88 mg, 1.2 mmol, 0.2 eq) in DCM (20 mL) was added oxalic dichloride (2.7 g, 21 mmol, 3.5 eq) at 0° C. and stirred at 40° C. for 0.5 h to give a yellow solution. The reaction mixture was concentrated under reduced pressure to give allyl 5-((dichlorophosphoryl)fluoromethyl)benzo[b]thiophene-2-carboxylate (2.2 g, crude) as a yellow solid. LCMS (ESI) m/z=717.1 (MeOH quench)

### Step 6: Preparation of Allyl 5-(fluoro((((S)-1-oxo-1-propoxypropan-2-yl)amino)(phenoxy)phosphoryl)methyl)benzo[b]thiophene-2-carboxylate

To a solution of allyl 5-((dichlorophosphoryl)fluoromethyl)benzo[b]thiophene-2-carboxylate (2.2 g, 6.0 mmol, 1 eq) in DCM (20 mL), the solution was under N₂ and cooled to 0° C., then phenol (0.56 g, 6.0 mmol, 1 eq) in DCM (10 mL) was added in it, ethylbis(propan-2-yl)amine (3.1 g, 24 mmol, 4 eq) in DCM (60 mL) was dropwise added in it and stirred at 0° C. for 5 min, 3,3,3-trifluoropropyl (2S)-2-aminopropanoate (0.91 g, 6.0 mmol, 1 eq) in DCM (20 mL) was added in it and stirred at 0° C. for 10 min to give a yellow solution. The reaction mixture was concentrated under reduced pressure to give a residue. The residue was purified by column chromatography to give allyl 5-(fluoro((((S)-1-oxo-1-propoxypropan-2-yl)amino)(phenoxy)phosphoryl)methyl)benzo[b]thiophene-2-carboxylate (1.1 g, 1.9 mmol, 33% yield) as a white solid. ¹H NMR (400 MHz, CDCl₃) δ 8.11-8.07 (m, 1H), 8.05-7.99 (m, 1H), 7.93-7.86 (m, 1H), 7.67-7.58 (m, 1H), 7.40-7.32 (m, 1H), 7.27-7.17 (m, 2H), 7.15-7.05 (m, 2H), 6.10-5.89 (m, 2H), 5.50-5.42 (m, 1H), 5.34 (dd, J=1.2, 10.4 Hz, 1H), 4.87 (d, J=5.6 Hz, 2H), 4.22-4.08 (m, 1H), 4.00-3.76 (m, 1H), 3.69-3.51 (m, 1H), 1.67-1.59 (m, 2H), 1.33-1.26 (m, 3H), 0.96-0.89 (m, 3H).

### Step 7: Preparation of 5-(fluoro((((S)-1-oxo-1-propoxypropan-2-yl)amino)(phenoxy)phosphoryl)methyl)benzo[b]thiophene-2-carboxylic acid

To a solution of allyl 5-(fluoro((((S)-1-oxo-1-propoxypropan-2-yl)amino)(phenoxy)phosphoryl)methyl)benzo[b]thiophene-2-carboxylate (1 g, 1.9 mmol, 1 eq) and pyrrolidine (82 mg, 1.2 mmol, 0.6 eq) in DCM (10 mL) was added Pd(PPh₃)₄(0.22 g, 0.19 mmol, 0.1 eq) and stirred at 25° C. for 1 h to give a yellow solution. The reaction mixture was concentrated under reduced pressure to give a residue. The crude was purified by reverse phase chromatography (TFA) then lyophilized to give 5-(fluoro((((S)-1-oxo-1-propoxypropan-2-yl)amino)(phenoxy)phosphoryl)methyl)benzo[b]thiophene-2-carboxylic acid (0.70 g, 1.5 mmol, 76% yield) as a white solid. ¹H NMR (400 MHz, CDCl₃) δ 8.15-7.91 (m, 1H), 7.90-7.79 (m, 2H), 7.70-7.51 (m, 1H), 7.46-7.29 (m, 3H), 7.25-7.15 (m, 2H), 6.15-5.98 (m, 1H), 4.24-4.05 (m, 3H), 1.79-1.58 (m, 2H), 1.41-1.22 (m, 3H), 0.96 (dd, J=12.4, 19.6 Hz, 3H).

### Step 8: Preparation of Perfluorophenyl 5-(fluoro((((S)-1-oxo-1-propoxypropan-2-yl)amino)(phenoxy)phosphoryl)methyl)benzo[b]thiophene-2-carboxylate

To a solution of 5-(fluoro((((S)-1-oxo-1-propoxypropan-2-yl)amino)(phenoxy)phosphoryl)methyl)benzo[b]thiophene-2-carboxylic acid (0.70 g, 1.5 mmol, 1 eq) and pyridine (0.57 g, 7.2 mmol, 5 eq) in DMF (10 mL) was added 2,3,4,5,6-pentafluorophenyl 2,2,2-trifluoroacetate (2.0 g, 7.2 mmol, 5 eq) and stirred at 25° C. for 0.5 h to give a yellow solution. The reaction mixture was diluted with water (50 mL) and extracted with EtOAc (50 mL×2), the combined organic layers were washed with saturated brine (40 mL×2), dried over Na₂SO₄, filtered and concentrated under reduced pressure to give a residue. The residue was purified by column chromatography to give perfluorophenyl 5-(fluoro((((S)-1-oxo-1-propoxypropan-2-yl)amino)(phenoxy)phosphoryl)methyl)benzo[b]thiophene-2-carboxylate (0.80 g, 1.2 mmol, 86% yield) as a white solid. LCMS (ESI) m/z=646.1 [M+H]⁺. ¹H NMR (400 MHz, CDCl₃) δ 8.38-8.30 (m, 1H), 8.15-8.07 (m, 1H), 8.02-7.91 (m, 1H), 7.78-7.63 (m, 1H), 7.43-7.34 (m, 1H), 7.31-7.28 (m, 1H), 7.25-7.17 (m, 1H), 7.16-7.06 (m, 2H), 6.13-5.89 (m, 1H), 4.11-4.01 (m, 2H), 3.70-3.51 (m, 1H), 1.72-1.59 (m, 3H), 1.32 (d, J=7.2 Hz, 2H), 0.99-0.87 (m, 3H).

### Synthesis of ((2-((perfluorophenoxy)carbonyl)benzo[b]thiophen-5-yl)methyl)phosphonic

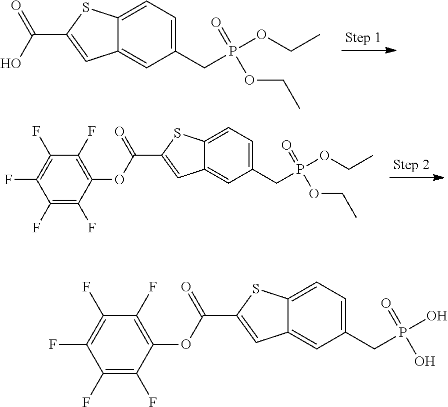

### Step 1: Preparation of ((2-((perfluorophenoxy)carbonyl)benzo[b]thiophen-5-yl)methyl)phosphonic acid

To a solution of 5-((diethoxyphosphoryl)methyl)benzo[b]thiophene-2-carboxylic acid (2 g, 6.1 mmol, 1 eq) in dimethylformamide (100 mL) was added pyridine (1.9 g, 24 mmol, 4 eq), the mixture was stirred at 0° C. for 5 min, then 2,3,4,5,6-pentafluorophenyl 2,2,2-trifluoroacetate (6.8 g, 24 mmol, 4 eq) was added to the mixture, the mixture was stirred at 25° C. for 15 min to give yellow solution. The mixture was diluted with water (50 mL) and extracted with EtOAc (50 mL×2), the combined organic layers were washed with saturated brine (40 mL×2), dried over Na₂SO₄, filtered and concentrated under reduced pressure to give a residue. The residue was purified by column chromatography to give perfluorophenyl 5-((diethoxyphosphoryl)methyl)benzo[b]thiophene-2-carboxylate (1.4 g, 2.9 mmol, 47% yield) as a yellow oil. LCMS (ESI) m/z=495.0

### Step 2: Preparation of ((2-((perfluorophenoxy)carbonyl)benzo[b]thiophen-5-yl)methyl)phosphonic acid

To a solution of perfluorophenyl 5-((diethoxyphosphoryl)methyl)benzo[b]thiophene-2-carboxylate (1.4 g, 2.8 mmol, 1 eq) in DCM (10 mL) was added trimethylsilyl iodide (2.3 g, 11 mmol, 4 eq), the mixture was stirred at 0° C. for 15 min to give yellow solution. The reaction mixture was concentrated under reduced pressure to give a residue. The residue was purified by reversed phase (TFA) to give ((2-((perfluorophenoxy)carbonyl)benzo[b]thiophen-5-yl)methyl)phosphonic acid (0.9 g, 2.1 mmol, 74% yield) as a white solid. LCMS (ESI) m/z=438.9 [M+H]⁺. ¹H NMR (400 MHz, CD₃OD) δ 8.41 (s, 1H), 8.01-7.94 (m, 2H), 7.60-7.55 (m, 1H), 3.32-3.32 (m, 1H), 3.26 (s, 1H).

### Synthesis of (fluoro(2-((perfluorophenoxy)carbonyl)benzo[b]thiophen-5-yl)methyl)phosphonic acid

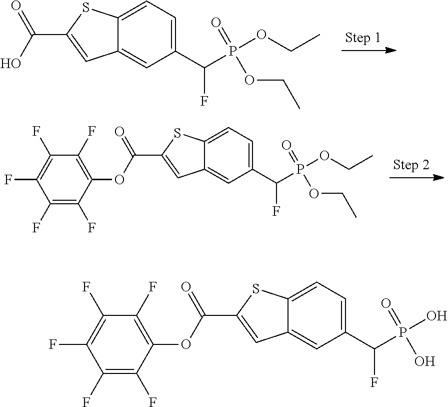

### Step 1: Preparation of Perfluorophenyl 5-((diethoxyphosphoryl)fluoromethyl)benzo[b]thiophene-2-carboxylate

To a solution of 5-[(diethoxyphosphoryl)(fluoro)methyl]-1-benzothiophene-2-carboxylic acid (5 g, 14.40 mmol, 1 eq) in piperidine (6.13 g, 72 mmol, 5 eq) was added 2,3,4,5,6-pentafluorophenyl 2,2,2-trifluoroacetate (12 g, 43.20 mmol, 3 eq), the mixture was stirred at 0° C. for 30 min to give a brown clean solution. The reaction mixture was concentrated under reduced pressure to give a residue. The residue was diluted with water (200 mL) and extracted with EtOAc (500 mL×2), the combined organic layers were washed with saturated brine (500 mL×2), dried over Na₂SO₄, filtered and concentrated under reduced pressure to give a residue. The residue was purified by flash silica gel chromatography to give perfluorophenyl 5-((diethoxyphosphoryl)fluoromethyl)benzo[b]thiophene-2-carboxylate (6.50 g, 11.70 mmol, 81.40%) as a yellow oil. LCMS (ESI) m/z=513.1 [M+H]⁺. ¹H NMR (400 MHz, CDCl₃) δ 8.35 (s, 1H), 8.08 (s, 1H), 7.98 (d, J=8.4 Hz, 1H), 7.67 (d, J=8.4 Hz, 1H), 5.93-5.77 (m, 1H), 4.22-4.17 (m, 1H), 4.16-4.13 (m, 1H), 4.13-4.11 (m, 1H), 4.10-4.06 (m, 1H), 1.34-1.31 (m, 3H), 1.30-1.27 (m, 3H).

### Step 2: Preparation of (fluoro(2-((perfluorophenoxy)carbonyl)benzo[b]thiophen-5-yl)methyl)phosphonic acid

To a solution of perfluorophenyl 5-((diethoxyphosphoryl)fluoromethyl)benzo[b]thiophene-2-carboxylate (6 g, 12 mmol, 1 eq) in DCM (60 mL) was dropwise TMSI (9.3 g, 47 mmol, 4 eq) at 0 C, the mixture was stirred at 0° C. for 1 h. The reaction mixture was concentrated under reduced pressure to give a yellow oil. The oil was purified by reversed phase (TFA) to lyophilized to give (fluoro(2-((perfluorophenoxy)carbonyl)benzo[b]thiophen-5-yl)methyl)phosphonic acid (3.0 g, 6.6 mmol, 56% yield) as a white solid. LCMS (ESI) m/z=457.2 [M+H]⁺. ¹H NMR (400 MHz, CD₃OD) δ 8.48 (s, 1H), 8.17 (s, 1H), 8.07 (d, J=8.4 Hz, 1H), 7.73 (d, J=8.4 Hz, 1H), 5.97-5.78 (m, 1H).

### Synthesis of (1-(2-((perfluorophenoxy)carbonyl)benzo[b]thiophen-5-yl)ethyl)phosphonic acid

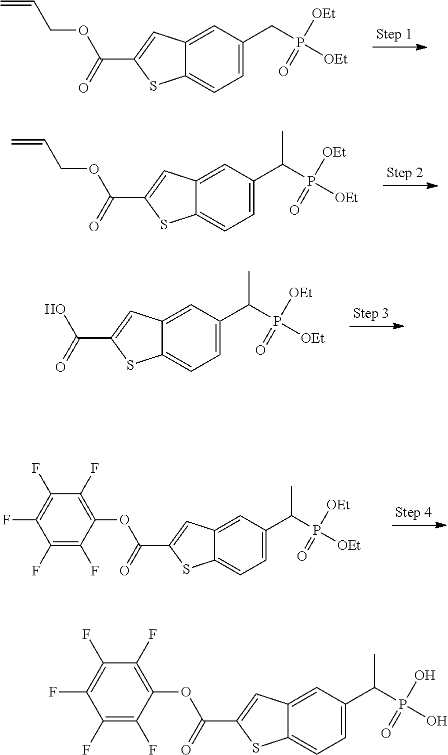 [SMILES: C=CCOC(=O)c1cc2cc(CP(=O)(OCC)OCC)ccc2s1 ; C=CCOC(=O)c1cc2cc(C(C)P(=O)(OCC)OCC)ccc2s1 ; CCOP(=O)(OCC)C(C)c1ccc2sc(C(=O)O)cc2c1 ; CCOP(=O)(OCC)C(C)c1ccc2sc(C(=O)Oc3c(F)c(F)c(F)c(F)c3F)cc2c1 ; CC(c1ccc2sc(C(=O)Oc3c(F)c(F)c(F)c(F)c3F)cc2c1)P(=O)(O)O]

### Step 1: Preparation of Allyl 5-(1-(diethoxyphosphoryl)ethyl)benzo[b]thiophene-2-carboxylate

To a solution of allyl 5-((diethoxyphosphoryl)methyl)benzo[b]thiophene-2-carboxylate (1.4 g, 3.80 mmol, 1 eq.) and CH₃I (809 mg, 5.7 mmol, 1.5 eq.) in THF (3 mL) was added LiHMDS (3.99 mmol, 1.05 eq.) dropwise at −78° C. for 10 min. The reaction mixture was stirred at room temperature for 2 h. After completion, the reaction mixture was quenched with NH₄Cl (aq, sat.) and extracted with EtOAc (20 mL×2). The organic layers were combined and washed with brine (10 mL), dried over anhydrous Na₂SO₄, and concentrated under reduced pressure. The residue was purified by Biotage® C18 column chromatography to afford allyl 5-(1-(diethoxyphosphoryl)ethyl)benzo[b]thiophene-2-carboxylate (520 mg, 1.36 mmol, 36%) as a colorless oil. LCMS (ESI) m/z=383 [M+H]⁺.

### Step 2: Preparation of 5-(1-(diethoxyphosphoryl)ethyl)benzo[b]thiophene-2-carboxylic acid

To a solution of allyl 5-(1-(diethoxyphosphoryl)ethyl)benzo[b]thiophene-2-carboxylate (120 mg, 0.31 mmol, 1.0 eq) in DCM (5 mL) were added Pd(PPh₃)₄(36 mg, 31 μmol, 0.1 eq.) and pyrrolidine (22 mg, 0.31 mmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 5-(1-(diethoxyphosphoryl)ethyl)benzo[b]thiophene-2-carboxylic acid (95 mg, 0.28 mmol, 90%) as a colorless oil. LCMS (ESI): m/z=343 [M+H]⁺.

### Step 3: Preparation of Perfluorophenyl 5-(1-(diethoxyphosphoryl)ethyl)benzo[b]thiophene-2-carboxylate

Oxalyl chloride (127 mg, 1.05 mmol, 1.5 eq.) was added dropwise to the solution of 5-(1-(diethoxyphosphoryl)ethyl)benzo[b]thiophene-2-carboxylic acid (240 mg, 0.70 mmol, 1.0 eq.) in dry DCM (10 mL) and DMF (1 drop) at 0° C. The reaction mixture was stirred at room temperature for an additional 2 h. After completion, the excess oxalyl chloride and solvent were removed under reduced pressure. The residue was re-dissolved in anhydrous DCM (10 mL), then added to a mixture of PFPOH (193 mg, 1.05 mmol, 1.5 eq.) and TEA (212 mg, 2.1 mmol, 3 eq.) in anhydrous DCM (10 mL). The reaction was stirred for an additional 0.5 h. After completion, the reaction mixture was concentrated under reduced pressure. The residue was purified by flash column chromatography on silica gel to afford perfluorophenyl 5-(1-(diethoxyphosphoryl)ethyl)benzo[b]thiophene-2-carboxylate (320 mg, 0.63 mmol, 90%) as a yellow solid. LCMS (ESI): m/z=509 [M+H]⁺.

### Step 1: Preparation of (1-(2-((perfluorophenoxy)carbonyl)benzo[b]thiophen-5-yl)ethyl)phosphonic acid

To a solution perfluorophenyl 5-(1-(diethoxyphosphoryl)ethyl)benzo[b]thiophene-2-carboxylate (320 mg, 0.63 mmol, 1 eq.) in DCM (5 mL) was added TMSBr (964 mg, 6.3 mmol, 10 eq.). The mixture was stirred at room temperature for 2 h. After completion, the reaction mixture was concentrated under reduced pressure. The residue was purified by flash column chromatography on silica gel to afford (1-(2-((perfluorophenoxy)carbonyl)benzo[b]thiophen-5-yl)ethyl)phosphonic acid (230 mg, 0.51 mmol, 81%) as a white solid. LCMS (ESI): m/z=453 [M+H]⁺.

### Synthesis of (1-(7-((perfluorophenoxy)carbonyl)naphthalen-2-yl)ethyl)phosphonic acid

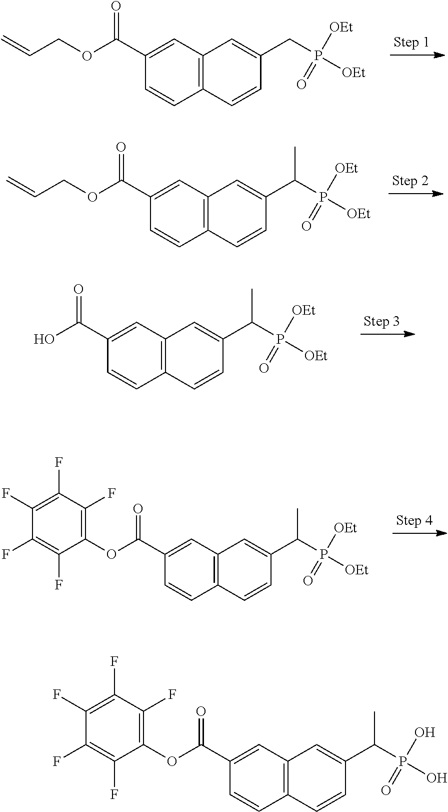

### Step 1: Preparation of Allyl 7-(1-(diethoxyphosphoryl)ethyl)-2-naphthoate

To a solution of allyl 7-((diethoxyphosphoryl)methyl)-2-naphthoate (2.0 g, 5.52 mmol, 1 eq.) and CH₃I (1.18 g, 8.28 mmol, 1.5 eq.) in THF (3 mL) was added LiHMDS (5.80 mmol, 1.05 eq.) dropwise at −78° C. for 10 min. The reaction mixture was stirred at room temperature for 2 h. After completion, the reaction mixture was quenched with NH₄Cl (aq, sat.) and extracted with EtOAc (20 mL×2). The organic layers were combined and washed with brine (10 mL), dried over anhydrous Na₂SO₄, and concentrated under reduced pressure. The residue was purified by Biotage® C18 column chromatography to afford allyl 7-(1-(diethoxyphosphoryl)ethyl)-2-naphthoate (1.05 g, 2.79 mmol, 51%) as a colorless oil. LCMS (ESI) m/z=377 [M+H]⁺.

### Step 2: Preparation of 7-(1-(diethoxyphosphoryl)ethyl)-2-naphthoic acid

To a solution of allyl 7-(1-(diethoxyphosphoryl)ethyl)-2-naphthoate (300 mg, 0.80 mmol, 1.0 eq) in DCM (5 mL) were added Pd(PPh₃)₄(92 mg, 80 μmol, 0.1 eq.) and pyrrolidine (57 mg, 0.80 mmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 7-(1-(diethoxyphosphoryl)ethyl)-2-naphthoic acid (240 mg, 0.71 mmol, 89%) as a colorless oil. LCMS (ESI): m/z=337 [M+H]⁺.

### Step 3: Preparation of Perfluorophenyl 7-(1-(diethoxyphosphoryl)ethyl)-2-naphthoate

Oxalyl chloride (134 mg, 1.07 mmol, 1.5 eq.) was added dropwise to the solution of 7-(1-(diethoxyphosphoryl)ethyl)-2-naphthoic acid (240 mg, 0.71 mmol, 1.0 eq.) in dry DCM (10 mL) and DMF (1 drop) at 0° C. The reaction mixture was stirred at room temperature for an additional 2 h. After completion, the excess oxalyl chloride and solvent were removed under reduced pressure. The residue was re-dissolved in anhydrous DCM (10 mL), then added to a mixture of PFPOH (197 mg, 1.07 mmol, 1.5 eq.) and TEA (212 mg, 2.1 mmol, 3 eq.) in anhydrous DCM (10 mL). The reaction was stirred for an additional 0.5 h. After completion, the reaction mixture was concentrated under reduced pressure. The residue was purified by flash column chromatography on silica gel to afford perfluorophenyl 7-(1-(diethoxyphosphoryl)ethyl)-2-naphthoate (320 mg, 0.64 mmol, 90%) as a yellow solid. LCMS (ESI): m/z=503 [M+H]⁺.

### Step 4: Preparation of (1-(7-((perfluorophenoxy)carbonyl)naphthalen-2-yl)ethyl)phosphonic acid

To a solution perfluorophenyl 7-(1-(diethoxyphosphoryl)ethyl)-2-naphthoate (320 mg, 0.64 mmol, 1 eq.) in DCM (5 mL) was added TMSBr (979 mg, 6.4 mmol, 10 eq.). The mixture was stirred at room temperature for 2 h. After completion, the reaction mixture was concentrated under reduced pressure. The residue was purified by flash column chromatography on silica gel to afford (1-(7-((perfluorophenoxy)carbonyl)naphthalen-2-yl)ethyl)phosphonic acid (220 mg, 0.49 mmol, 77%) as a white solid. LCMS (ESI): m/z=447 [M+H]⁺.

### Synthesis of (methoxy(2-((perfluorophenoxy)carbonyl)benzo[b]thiophen-5-yl)methyl)phosphonic acid

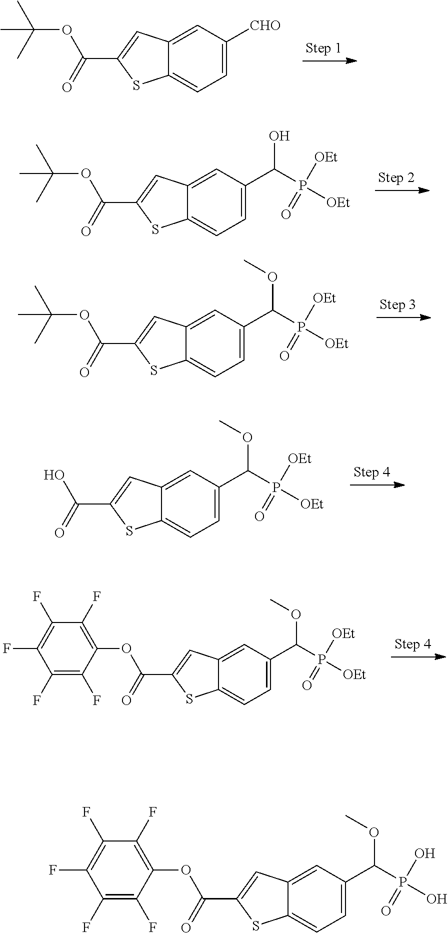

### Step 1: Preparation of Tert-Butyl 5-((diethoxyphosphoryl)(hydroxy)methyl)benzo[b]thiophene-2-carboxylate

To a solution of tert-butyl 5-formylbenzo[b]thiophene-2-carboxylate (50 mg, 190 μmol, 1 eq.) and diethyl phosphonate (28.8 mg, 209 μmol, 1.1 eq.) in DCM (15 mL) was added DBU (2.89 mg, 19.0 μmol, 0.1 eq.). The reaction solution was stirred at room temperature for 18 h. After completion, the reaction mixture was concentrated under reduced pressure. The residue was purified by flash column chromatography on silica gel to afford tert-butyl 5-((diethoxyphosphoryl)(hydroxy)methyl)benzo[b]thiophene-2-carboxylate (60.0 mg, 149 μmol, 79%) as a colorless oil. LCMS (ESI) m/z=401.2 [M+H]⁺.

### Step 2: Preparation of tert-butyl 5-((diethoxyphosphoryl)(methoxy)methyl)benzo[b]thiophene-2-carboxylate

To a solution of tert-butyl 5-((diethoxyphosphoryl)(hydroxy)methyl)benzo[b]thiophene-2-carboxylate (150 mg, 374 μmol, 1 eq.) and CH₃I (79.6 mg, 561 μmol, 1.5 eq.) in DMF (3 mL) was added LiHMDS (65.5 mg, 392 μmol, 1.05 eq.) dropwise at −18° C. for 10 min. The reaction mixture was stirred at room temperature for 2 h. After completion, the reaction mixture was quenched with NH₄Cl (aq, sat.) and extracted with EtOAc (20 mL×2). The organic layers were combined and washed with brine (100 mL), dried over anhydrous Na₂SO₄, and concentrated under reduced pressure. The residue was purified by Biotage® C18 column chromatography to afford tert-butyl 5-((diethoxyphosphoryl)(methoxy)methyl)benzo[b]thiophene-2-carboxylate (40.0 mg, 96.5 μmol, 26%) as a colorless oil. LCMS (ESI) m/z=415.1 [M+H]⁺. ¹H NMR (400 MHz, CDCl₃) δ 7.96 (d, J=16.2 Hz, 2H), 7.87 (d, J=8.4 Hz, 1H), 7.55 (d, J=8.3 Hz, 1H), 4.64 (d, J=15.4 Hz, 1H), 4.18-3.88 (m, 4H), 3.43 (s, 3H), 1.63 (s, 9H), 1.32-1.19 (m, 6H).

### Step 3: Preparation of 5-((diethoxyphosphoryl)(methoxy)methyl)benzo[b]thiophene-2-carboxylic acid

To a solution of tert-butyl 5-((diethoxyphosphoryl)(methoxy)methyl)benzo[b]thiophene-2-carboxylate (40 mg, 96.5 μmol, 1 eq.) in DCM (3 mL) was added TFA (1 mL). The mixture was stirred at room temperature for 2 h. After completion, the reaction mixture was concentrated under reduced pressure to afford 5-((diethoxyphosphoryl)(methoxy)methyl)benzo[b]thiophene-2-carboxylic acid (30 mg, quant.) as a colorless oil, which was used in next step directly without further purification. LCMS (ESI) m/z=359 [M+H]⁺.

### Step 4: Preparation of Perfluorophenyl 5-((diethoxyphosphoryl)(methoxy)methyl)benzo[b]thiophene-2-carboxylate

Oxalyl chloride (15.8 mg, 125 μmol, 1.5 eq.) was added dropwise to the solution of 5-((diethoxyphosphoryl)(methoxy)methyl)benzo[b]thiophene-2-carboxylic acid (30 mg, 83.7 μmol, 1.0 eq.) in dry DCM (10 mL) and DMF (2 drops) at 0° C. The reaction mixture was stirred at room temperature for an additional 2 h. After completion, the excess oxalyl chloride and solvent were removed under reduced pressure. The residue was re-dissolved in anhydrous DCM (10 mL), then added to a mixture of PFPOH (23.0 mg, 125 μmol, 1.5 eq.) and TEA (25.3 mg, 251 μmol, 3 eq.) in anhydrous DCM (10 mL). The reaction was stirred for an additional 0.5 h. After completion, the reaction mixture was concentrated under reduced pressure. The residue was purified by flash column chromatography on silica gel to afford perfluorophenyl 5-((diethoxyphosphoryl)(methoxy)methyl)benzo[b]thiophene-2-carboxylate (38.0 mg, 72.4 μmol, 87%) as a yellow solid. LCMS (ESI): m/z=525.0 [M+H]⁺.

### Step 5: Preparation of (methoxy(2-((perfluorophenoxy)carbonyl)benzo[b]thiophen-5-yl)methyl)phosphonic acid

To a solution perfluorophenyl 5-((diethoxyphosphoryl)(methoxy)methyl)benzo[b]thiophene-2-carboxylate (38.0 mg, 72.4 μmol, 1 eq.) in DCM (1 mL) was added TMSBr (110 mg, 0.72 mmol, 10 eq.). The mixture was stirred at room temperature for 2 h. After completion, the reaction mixture was concentrated under reduced pressure. The residue was purified by flash column chromatography on silica gel to afford (methoxy(2-((perfluorophenoxy)carbonyl)benzo[b]thiophen-5-yl)methyl)phosphonic acid (17 mg, 36 μmol, 50%) as a white solid. LCMS (ESI): m/z=469 [M+H]⁺.

### Synthesis of 5-((1S)-fluoro(phenoxy((S)-2-(propoxycarbonyl)pyrrolidin-1-yl)phosphoryl)methyl)benzo[b]thiophene-2-carboxylic acid

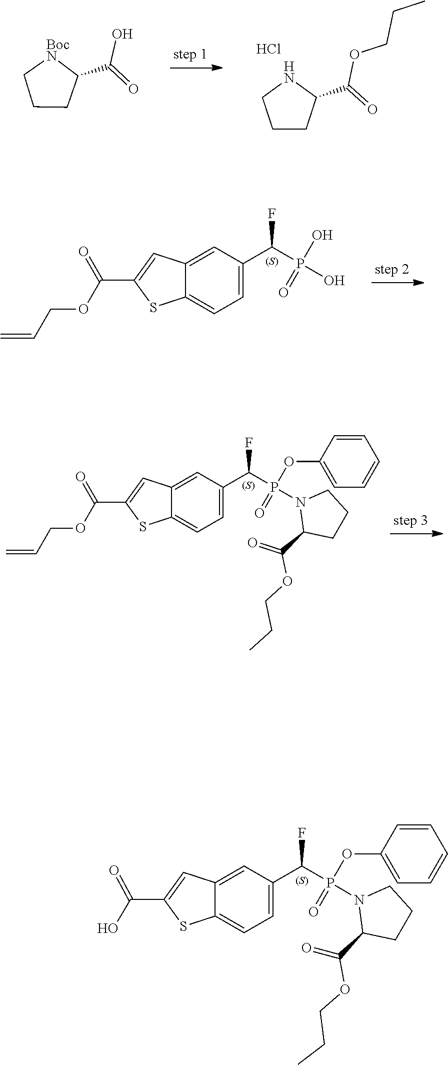

### Step 1: Preparation of propyl L-prolinate hydrochloride

Thionyl chloride (2.01 mL, 27.8 mmol, 3 eq) was added dropwise to a solution of (tert-butoxycarbonyl)-L-proline (2.00 g, 9.29 mmol, 1 eq) in propan-1-ol (20 mL, 332 μmol, eq) at 0° C. The mixture was allowed to warm to ambient temperature and then heated at 80° C. overnight. The reaction mixture was concentrated under reduced pressure to give crude propyl L-prolinate hydrochloride (1.85 g, 103%) as a brown oil. ¹H NMR (400 MHz, DMSO-d₆) δ 4.36-4.33 (m, 1H), 4.18-4.07 (m, 2H), 3.26-3.14 (m, 2H), 2.32-2.22 (m, 1H), 2.01-1.85 (m, 3H), 1.66-1.59 (m, 2H), 0.92-0.89 (m, 3H).

The following intermediates in Table 72 were prepared using the method described above in step 1 for the preparation of propyl L-prolinate hydrochloride and utilizing the appropriate starting materials and modifications.

TABLE 72

| Name | Structure | LCMS |
|---|---|---|
| propyl (S)-azetidine-2- carboxylate (HCl salt) | 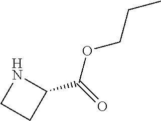 | 144.2 [M + H]⁺ |

### Step 2: Preparation of propyl (((S)-(2-((allyloxy)carbonyl)benzo[b]thiophen-5-yl)fluoromethyl)(phenoxy)phosphoryl)-L-prolinate

To a solution of (S)-((2-((allyloxy)carbonyl)benzo[b]thiophen-5-yl)fluoromethyl)phosphonic acid (500 mg, 1.51 mmol, 1 eq) and DMF (2 drops) in DCM (15 mL) was added dropwise oxalyl chloride (1.02 mL, 12.0 mmol, 8 eq). The reaction mixture was stirred 2 h at room temperature under N₂. The reaction mixture was concentrated under reduced pressure and dried completely under high vacuum for 30 min. to give a yellow solid. The yellow solid was diluted in DCM (15 mL, dried on Na₂SO₄) and cooled down to 0° C. A solution of phenol (134 mg, 1.43 mmol, 0.95 eq) and triethylamine (1.26 mL, 9.06 mmol, 6.0 eq) in DCM (5 mL, dried on Na₂SO₄) was slowly added onto the solution. The reaction mixture was stirred at 0° C. for 2 min. and then warmed up to room temperature and stirred for 18 h. A solution of propyl L-prolinate hydrochloride (584 mg, 3.02 mmol, 2 eq) in DCM (5 mL, dried on Na₂SO₄) was slowly added onto the orange solution. The reaction mixture was stirred at room temperature for 4 h. Water (1 mL) was added. The solvent was removed under reduced pressure. The crude residue was purified by reverse phase chromatography on a 150 g C₁₈ cartridge eluting with 5-100% MeCN in water. The pure fractions were then concentrated under reduced pressure to give propyl (((S)-(2-((allyloxy)carbonyl)benzo[b]thiophen-5-yl)fluoromethyl)(phenoxy)phosphoryl)-L-prolinate (320 mg, 38.8%) as a yellow oil. LCMS (ESI): m/z=546.2 [M+H]⁺. ¹H NMR (400 MHz, CDCl₃) δ 8.17-8.04 (m, 2H), 7.94-7.86 (m, 1H), 7.79-7.62 (m, 1H), 7.34-7.28 (m, 2H), 7.24-7.20 (m, 1H), 7.19-7.03 (m, 2H), 6.10-6.00 (m, 1H), 5.49-5.31 (m, 2H), 4.89-4.84 (m, 2H), 4.57-4.22 (m, 1H), 4.15-3.96 (m, 2H), 3.66-3.21 (m, 1H), 3.13-2.67 (m, 1H), 2.18-2.04 (m, 1H), 1.97-1.86 (m, 1H), 1.83-1.46 (m, 5H), 1.00-0.86 (m, 3H).

The following intermediates in Table 73 were prepared using the method described above in step 2 for the preparation of propyl (((S)-(2-((allyloxy)carbonyl)benzo[b]thiophen-5-yl)fluoromethyl)(phenoxy)phosphoryl)-L-prolinate and utilizing the appropriate starting materials and modifications.

TABLE 73

| Name | Structure | LCMS |
|---|---|---|
| propyl (2S)-1-(((S)-(2- ((allyloxy)carbonyl) benzo[b]thiophen-5-yl) fluoromethyl)(phenoxy)phosphoryl) azetidine-2-carboxylate | 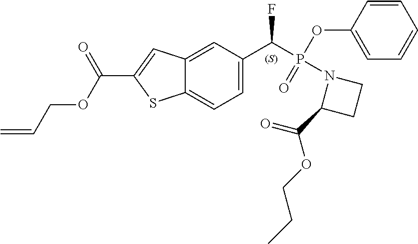 | 532.2 [M + H]⁺ |

### Step 3: Preparation of 5-((1S)-fluoro(phenoxy((S)-2-(propoxycarbonyl)pyrrolidin-1-yl)phosphoryl)methyl)benzo[b]thiophene-2-carboxylic acid

To a solution of propyl (((S)-(2-((allyloxy)carbonyl)benzo[b]thiophen-5-yl)fluoromethyl)(phenoxy)phosphoryl)-L-prolinate (320 mg, 586 μmol, 1 eq) in THF (6 mL) were added morpholine (251 μL, 2.92 mmol, 5 eq) and Pd(PPh₃)₄(13.5 mg, 11.7 μmol, 0.02 eq). The reaction mixture was stirred at room temperature for 1 h. The reaction mixture was concentrated to a volume of 2 mL under N₂. The crude product was purified by reverse phase chromatography on a 100 g C₁₈ cartridge eluting with 5-80% MeCN in water (with 0.1% formic acid). The combined fractions were concentrated under reduced pressure and then freeze dried to give 5-((1S)-fluoro(phenoxy((S)-2-(propoxycarbonyl)pyrrolidin-1-yl)phosphoryl)methyl)benzo[b]thiophene-2-carboxylic acid (172 mg) as a white solid which was re-purified by reverse phase chromatography on a 275 g C₁₈ cartridge eluting with 5-80% MeCN in water (with 0.1% formic acid). The combined fractions were concentrated under reduced pressure and then freeze dried to give 5-((1S)-fluoro(phenoxy((S)-2-(propoxycarbonyl)pyrrolidin-1-yl)phosphoryl)methyl)benzo[b]thiophene-2-carboxylic acid (75.0 mg, 25.3%) as a white solid. LCMS (ESI): m/z=506.0 [M+H]⁺. ¹H NMR (400 MHz, DMSO-d₆) δ 13.58 (br. s., 1H), 8.21-8.10 (m, 2H), 7.73-7.63 (m, 1H), 7.41-7.31 (m, 3H), 7.30-7.16 (m, 2H), 7.12-7.08 (m, 1H), 6.86-6.28 (m, 1H), 4.43-3.41 (m, 4H), 3.19-2.82 (m, 1H), 2.10-1.61 (m, 4H), 1.57-1.45 (m, 2H), 0.92-0.72 (m, 3H).

The following intermediates in Table 74 were prepared using the method described above in step 3 for the preparation of 5-((1S)-fluoro(phenoxy((S)-2-(propoxycarbonyl)pyrrolidin-1-yl)phosphoryl)methyl)benzo[b]thiophene-2-carboxylic acid and utilizing the appropriate starting materials and modifications.

TABLE 74

| Name | Structure | LCMS |
|---|---|---|
| 5-((1S)-fluoro(phenoxy((S)-2- (propoxycarbonyl)azetidin-1- yl)phosphoryl)methyl)benzo[b] thiophene-2-carboxylic acid | 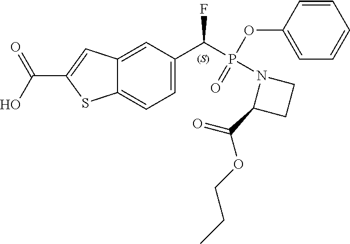 | 492.2 [M + H]⁺ |

### Synthesis of 5-((1R)-fluoro((((S)-1-(2-methoxy-2-methylpropoxy)-1-oxopropan-2-yl)amino)(phenoxy)phosphoryl)methyl)benzo[b]thiophene-2-carboxylic acid

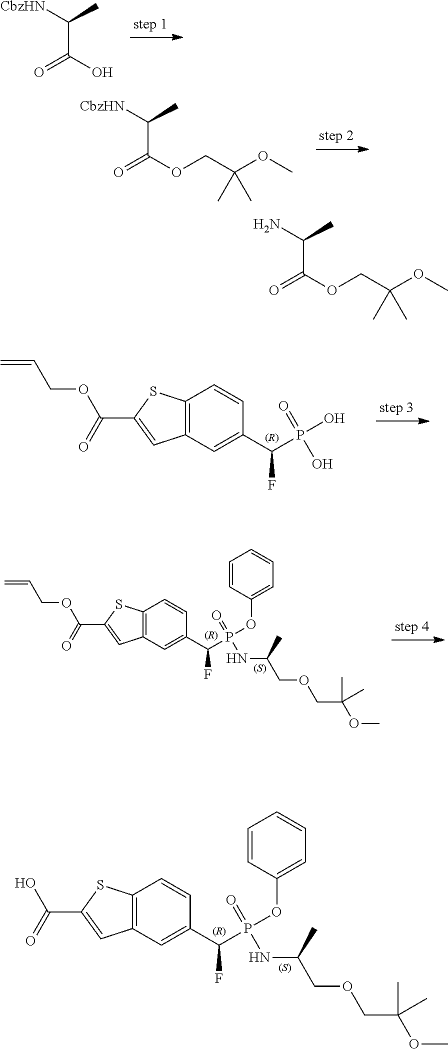

### Step 1: Preparation of 2-methoxy-2-methylpropyl ((benzyloxy)carbonyl)-L-alaninate

To a solution of ((benzyloxy)carbonyl)-L-alanine (1.64 g, 7.34 mmol, 1 eq.) and 2-methoxy-2-methylpropan-1-ol (916 mg, 8.80 mmol, 1.2 eq.) in ACN (20 ml) were added EDCI (1.68 g, 8.80 mmol, 1.2 eq.) and DMAP (1.07 g, 8.80 mmol, 1.2 eq.). The mixture was stirred at 25° C. for 16 h. After completion, the reaction mixture was diluted with H₂O (10 mL) and extracted with DCM (10 mL×3). The organic layers were combined and washed with brine (10 mL), dried over anhydrous Na₂SO₄, and concentrated under reduced pressure. The residue was purified by flash column chromatography on silica gel to afford 2-methoxy-2-methylpropyl ((benzyloxy)carbonyl)-L-alaninate (2, 1.63 g, 5.27 mmol, 72%) as a colorless oil. LCMS (ESI): m/z=310 [M+H]⁺.

The following intermediates in Table 75 were prepared using the method described above in step 1 for the preparation of 2-methoxy-2-methylpropyl ((benzyloxy)carbonyl)-L-alaninate and utilizing the appropriate starting materials and modifications.

TABLE 75

| Name | Structure | LCMS |
|---|---|---|
| (tetrahydro-2H-pyran- 4-yl)methyl ((benzyloxy)carbonyl)- L-alaninate | 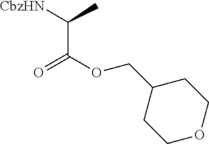 [SMILES: C[C@H](NC(=O)OCc1ccccc1)C(=O)OCC1CCOCC1] | 322 [M + H]⁺ |
| pyridazin-3-ylmethyl ((benzyloxy)carbonyl)- L-alaninate | 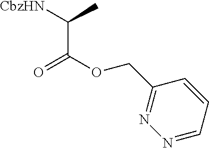 | 282 [M + H]⁺ |
| oxetan-3-ylmethyl ((benzyloxy)carbonyl)-L- alaninate | 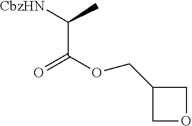 [SMILES: C[C@H](NC(=O)OCc1ccccc1)C(=O)OCC1COC1] | 294 [M + H]⁺ |
| (3-methyloxetan- 3-yl)methyl ((benzyloxy)carbonyl)- L-alaninate | 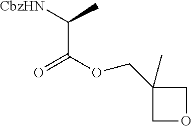 [SMILES: C[C@H](NC(=O)OCc1ccccc1)C(=O)OCC1(C)COC1] | 308 [M + H]⁺ |

### Step 2 N Preparation of 2-methoxy-2-methylpropyl L-alaninate

To a solution of 2-methoxy-2-methylpropyl ((benzyloxy)carbonyl)-L-alaninate (1.5 g, 4.84 mmol, 1.0 eq.) in EtOAc (15 ml) was added Pd/C (150 mg) under nitrogen. The suspension was degassed under vacuum and purged with H₂ several times. The resulting mixture was stirred at room temperature for 14 h. After completion, the suspension was filtered through a pad of Celite®, the filter cake was washed with MeOH (20 mL). The combined filtrates were concentrated to dryness to give 2-methoxy-2-methylpropyl L-alaninate (640 mg, 3.65 mmol, 75%) as a colorless oil. LCMS (ESI): m/z=176 [M+H]⁺.

The following intermediates in Table 76 were prepared using the method described above in step 2 for the preparation of 2-methoxy-2-methylpropyl ((benzyloxy)carbonyl)-L-alaninate and utilizing the appropriate starting materials and

TABLE 76

| Name | Structure | LCMS |
|---|---|---|
| (tetrahydro-2H-pyran- 4-yl)methy L-alaninate | 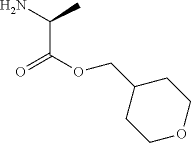 | 188 [M + H]⁺ |
| pyridazin-3-ylmethyl L-alaninate | 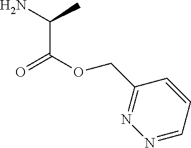 | 182 [M + H]⁺ |
| oxetan-3-ylmethyl L-alaninate | 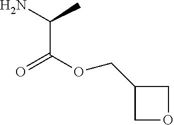 | 160 [M + H]⁺ |
| (3-methyloxetan-3-yl)methyl L-alaninate | 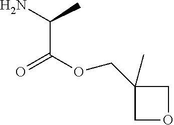 | 147.1 [M + H]⁺ |

### Step 3: Preparation of Allyl 5-((1R)-fluoro((((S)-1-(2-methoxy-2-methylpropoxy)-1-oxopropan-2-yl)amino)(phenoxy)phosphoryl)methyl)benzo[b]thiophene-2-carboxylate

Oxalyl chloride (576 mg, 4.54 mmol, 5 eq.) was added dropwise to the solution of (R)-((2-((allyloxy)carbonyl)benzo[b]thiophen-5-yl)fluoromethyl)phosphonic acid (300 mg, 0.91 mmol, 1 eq.) in dry DCM (10 mL) and DMF (1 drop) at 25° C. The reaction mixture was stirred at 40° C. for an additional 0.5 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (10 mL), then added to a mixture of phenol (88.8 mg, 0.94 mmol, 1.05 eq.) and TEA (908 mg, 8.98 mmol, 10.0 eq.) in anhydrous DCM (5 mL) at 0° C. The reaction was stirred for 0.5 h. Then 2-methoxy-2-methylpropyl L-alaninate (313 mg, 1.79 mmol, 2 eq.) was added at 0° C. and the resulting mixture was stirred at 20° C. for 1 h under N₂. After completion, the reaction mixture was concentrated under reduced pressure. The residue was purified by Biotage® C18 column chromatography to give allyl 5-((1R)-fluoro((((S)-1-(2-methoxy-2-methylpropoxy)-1-oxopropan-2-yl)amino)(phenoxy)phosphoryl)methyl)benzo[b]thiophene-2-carboxylate (45.0 mg, 80 μmol, 9%) as a yellow solid. LCMS (ESI): m/z=564.0 [M+H]⁺.

The following intermediates in Table 77 were prepared using the method described above in step 3 for the preparation of allyl 5-((1R)-fluoro((((S)-1-(2-methoxy-2-methylpropoxy)-1-oxopropan-2-yl)amino)(phenoxy)phosphoryl)methyl)benzo[b]thiophene-2-carboxylate and utilizing the appropriate starting materials and modifications.

TABLE 77

| Name | Structure | LCMS |
|---|---|---|
| allyl 5-((1R)-fluoro((((S)-1-oxo-1- ((tetrahydro-2H-pyran-4-yl)methoxy)propan- 2-yl)amino)(phenoxy)phosphoryl)methyl) benzo[b]thiophene-2-carboxylate | 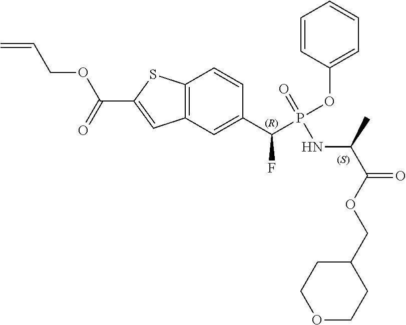 | 576 [M + H]⁺ |
| allyl 5-((1R)-fluoro((((S)-1-oxo-1-(pyridazin- 3-ylmethoxy)propan-2- yl)amino)(phenoxy)phosphoryl)methyl) benzo[b]thiophene-2-carboxylate | 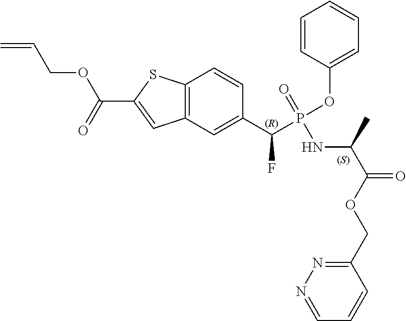 | 570 [M + H]⁺ |
| allyl 5-((1R)-fluoro((((S)-1-(oxetan-3- ylmethoxy)-1-oxopropan-2- yl)amino)(phenoxy)phosphoryl)methyl) benzo[b]thiophene-2-carboxylate | 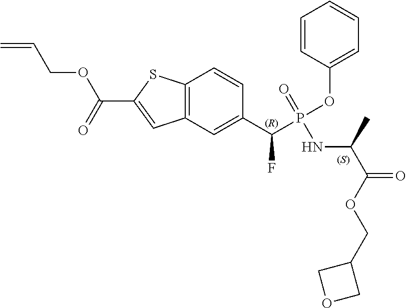 | 548 [M + H]⁺ |
| allyl 5-((1R)-fluoro((((S)-1-((3-methyloxetan- 3-yl)methoxy)-1-oxopropan-2- yl)amino)(phenoxy)phosphoryl)methyl) benzo[b]thiophene-2-carboxylate | 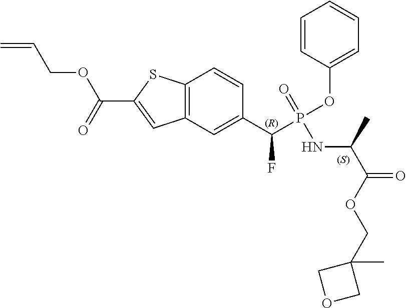 | 562 [M + H]⁺ |

### Step 4: Preparation of 5-((1R)-fluoro((((S)-1-(2-methoxy-2-methylpropoxy)-1-oxopropan-2-yl)amino)(phenoxy)phosphoryl)methyl)benzo[b]thiophene-2-carboxylic acid

To a solution of allyl 5-((1R)-fluoro((((S)-1-(2-methoxy-2-methylpropoxy)-1-oxopropan-2-yl)amino)(phenoxy)phosphoryl)methyl)benzo[b]thiophene-2-carboxylate (45.0 mg, 80 μmol, 1.0 eq) in DCM (6 mL) were added Pd(PPh₃)₄(5 mg, 8 μmol, 0.1 eq.) and pyrrolidine (6 mg, 80 μmol, 1.0 eq.). The mixture was purged and degassed with N₂ (three times), then stirred at 25° C. for 1 hr. After completion, the mixture was concentrated under reduced pressure. The residue was purified by Biotage® C18 column chromatography to give 5-((1R)-fluoro((((S)-1-(2-methoxy-2-methylpropoxy)-1-oxopropan-2-yl)amino) (phenoxy)phosphoryl)methyl)benzo[b]thiophene-2-carboxylic acid (40 mg, 76 μmol, 95%) as a colorless oil. LCMS (ESI): m/z=524 [M+H]⁺.

The following intermediates in Table 78 were prepared using the method described above in step 4 for the preparation of 5-((1R)-fluoro((((S)-1-(2-methoxy-2-methylpropoxy)-1-oxopropan-2-yl)amino)(phenoxy)phosphoryl)methyl)benzo[b]thiophene-2-carboxylic acid and

TABLE 78

| Name | Structure | LCMS |
|---|---|---|
| 5-((1R)-fluoro((((S)-1-oxo-1-((tetrahydro-2H- pyran-4-yl)methoxy)propan-2- yl)amino)(phenoxy)phosphoryl)methyl)benzo [b]thiophene-2-carboxylic acid | 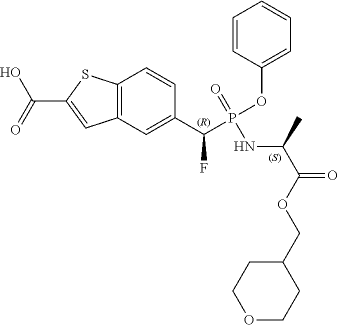 | 536 [M + H]⁺ |
| 5-((1R)-fluoro((((S)-1-oxo-1-(pyridazin-3- ylmethoxy)propan-2- yl)amino)(phenoxy)phosphoryl)methyl)benzo [b]thiophene-2-carboxylic acid | 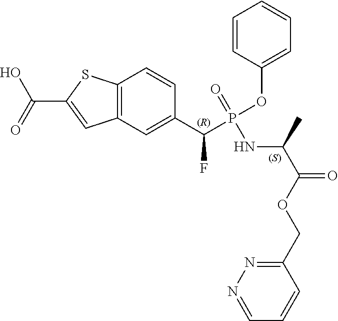 | 530 [M + H]⁺ |
| 5-((1R)-fluoro((((S)-1-(oxetan-3-ylmethoxy)- 1-oxopropan-2- yl)amino)(phenoxy)phosphoryl)methyl)benzo [b]thiophene-2-carboxylic acid | 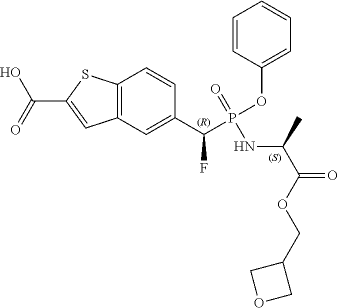 | 508 [M + H]⁺ |
| 5-((1R)-fluoro((((S)-1-((3-methyloxetan-3- yl)methoxy)-1-oxopropan-2- yl)amino)(phenoxy)phosphoryl)methyl) benzo[b]thiophene-2-carboxylic acid | 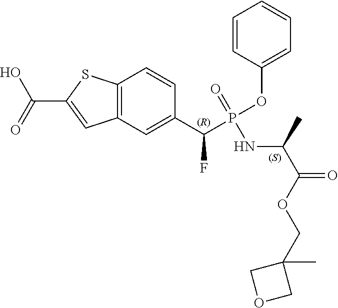 | 522 [M + H]⁺ |

### Synthesis of 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylic acid

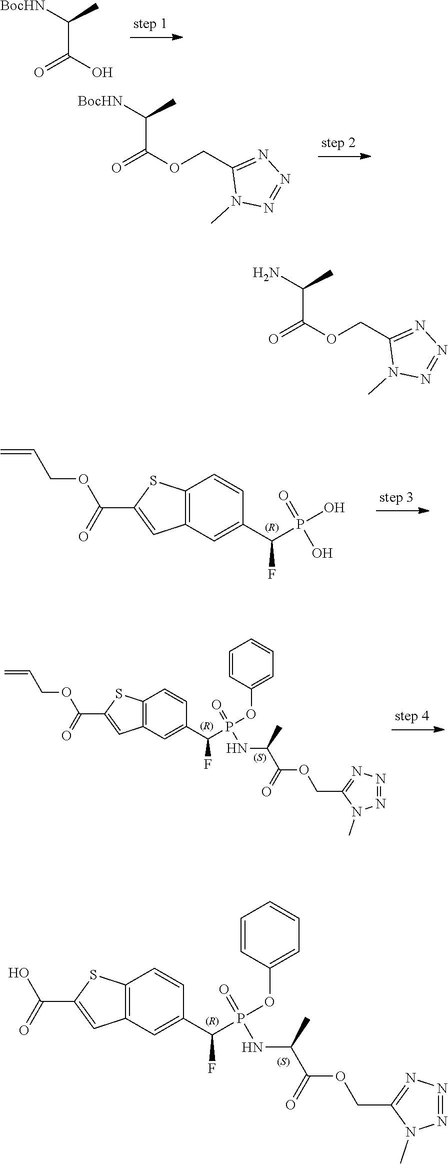

### Step 1: Preparation of (1-methyl-1H-tetrazol-5-yl)methyl (tert-butoxycarbonyl)-L-alaninate

To a solution of (tert-butoxycarbonyl)-L-alanine (1 g, 5.28 mmol, 1 eq.) and (1-methyl-1H-1,2,3,4-tetrazol-5-yl)methanol (903 mg, 7.92 mmol, 1.5 eq.) in DCM (15 ml) were added T₃P (50%) (5.02 g, 15.8 mmol, 3 eq.), DMAP (32.2 mg, 264 μmol, 0.05 eq.) and 4-methylmorpholine (1.59 g, 15.8 mmol, 3 eq.). The mixture was stirred at room temperature for 2 h. After completion, the reaction mixture was diluted with H₂O (10 mL) and extracted with DCM (10 mL×3). The organic layers were combined and washed with brine (10 mL), dried over anhydrous Na₂SO₄, and concentrated under reduced pressure. The residue was purified by flash column chromatography on silica gel to afford (1-methyl-1H-tetrazol-5-yl)methyl (tert-butoxycarbonyl)-L-alaninate (900 mg, 3.15 mmol, 60%) as a colorless oil. LCMS (ESI): m/z=286 [M+H]⁺.

The following intermediates in Table 79 were prepared using the method described above in step 1 for the preparation of (1-methyl-1H-tetrazol-5-yl)methyl (tert-butoxycarbonyl)-L-alaninate and utilizing the appropriate starting materials and

TABLE 79

| Name | Structure | LCMS | NMR |
|---|---|---|---|
| (4-methyl-4H-1,2,4-triazol-3- yl)methyl (tert-butoxycarbonyl)- L-alaninate | 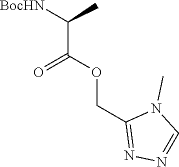 [SMILES: C[C@H](NC(=O)OC(C)(C)C)C(=O)OCc1nncn1C] | 285 [M + H]⁺ | |
| 2-methoxyethyl N-(tert- butoxycarbonyl)-O-methyl-L- serinate | 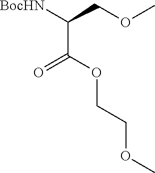 [SMILES: COCCOC(=O)[C@H](COC)NC(=O)OC(C)(C)C] | 278 [M + H]⁺ | ¹H NMR (400 MHz, DMSO-d₆) δ 7.19-7.07 (m, 1H), 4.28-4.10 (m, 3H), 3.61-3.46 (m, 4H), 3.28-3.19 (m, 6H), 1.38 (s, 9H). |
| 2-methoxyethyl (tert- butoxycarbonyl)-L-alaninate | 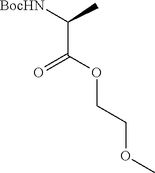 [SMILES: COCCOC(=O)[C@H](C)NC(=O)OC(C)(C)C] | 248 [M + H]⁺ | |
| propyl (tert-butoxycarbonyl)-L- alaninate | 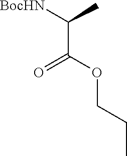 [SMILES: CCCOC(=O)[C@H](C)NC(=O)OC(C)(C)C] | | |
| propyl N-(tert-butoxycarbonyl)- O-methyl-L-serinate | 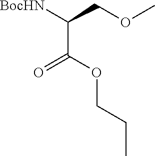 [SMILES: CCCOC(=O)[C@H](COC)NC(=O)OC(C)(C)C] | 262 [M + H]⁺ | |
| pyridin-2-ylmethyl (tert- butoxycarbonyl)-L-alaninate | 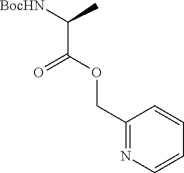 [SMILES: C[C@H](NC(=O)OC(C)(C)C)C(=O)OCc1ccccn1] | 281 [M + H]⁺ | |
| isobutyl (tert-butoxycarbonyl)- L-alaninate | 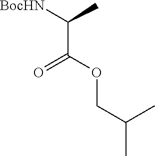 [SMILES: CC(C)COC(=O)[C@H](C)NC(=O)OC(C)(C)C] | 246 [M + H]⁺ | |
| cyclopropyl (tert- butoxycarbonyl)-L-alaninate | 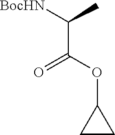 [SMILES: C[C@H](NC(=O)OC(C)(C)C)C(=O)OC1CC1] | 230 [M + H]⁺. | |
| neopentyl (tert-butoxycarbonyl)- L-alaninate | 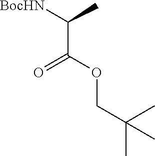 [SMILES: C[C@H](NC(=O)OC(C)(C)C)C(=O)OCC(C)(C)C] | 260 [M + H]⁺ | |

### Step 2: Preparation of (1-methyl-1H-tetrazol-5-yl)methyl L-alaninate

A solution of (1-methyl-H-tetrazol-5-yl)methyl (tert-butoxycarbonyl)-L-alaninate (600 mg, 2.10 mmol, 1 eq) in HCl/EA (10 mL) was stirred at room temperature for 1 hr. After completion, the reaction mixture was concentrated under reduced pressure to give crude (1-methyl-1H-tetrazol-5-yl)methyl L-alaninate (390 mg, quant.)(HCl salt) as a white solid, which was used in next step directly without further purification. LCMS (ESI): m/z 186 [M+H]⁺.

The following intermediates in Table 80 were prepared using the method described above in step 2 for the preparation of (1-methyl-1H-tetrazol-5-yl)methyl L-alaninate and utilizing the appropriate starting materials and modifications.

TABLE 80

| Name | Structure | LCMS | NMR |
|---|---|---|---|
| (4-methyl-4H-1,2,4-triazol-3- yl)methyl L-alaninate (HCl Salt) | 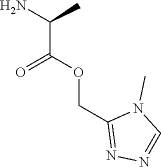 | 185 [M + H]⁺ | |
| 2-methoxyethyl O-methyl-L- serinate (HCl salt) | 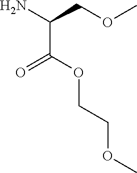 | | ¹H NMR (400 MHz, DMSO-d₆) δ 8.84 (s, 2H), 4.29-4.22 (m, 2H), 3.87-3.75 (m, 2H), 3.58-3.56 (m, 3H), 3.31-3.25 (m, 6H) |
| 2-methoxyethyl L-alaninate (HCl salt) | 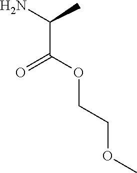 | 148 [M + H]⁺ | |
| propyl L-alaninate (HCl salt) | 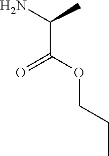 | | |
| propyl O-methyl-L-serinate (HCl salt) | 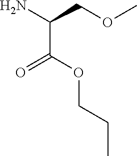 | 162 [M + H]⁺ | |
| pyridin-2-ylmethyl L-alaninate (HCl salt) | 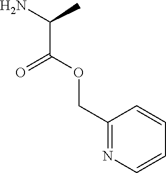 | 181 [M + H]⁺ | |
| isobutyl L-alaninate (HCl salt) | 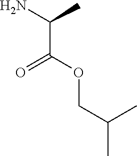 | 146 [M + H]⁺ | |
| cyclopropyl L-alaninate (HCl salt) | 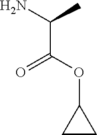 | 130 [M + H]⁺ | |
| benzyl L-alaninate (HCl salt) | 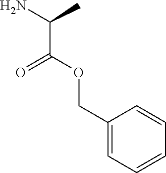 | 180 [M + H]⁺ | |
| neopentyl L-alaninate (HCl salt) | 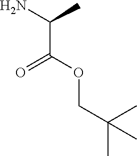 | 160 [M + H]⁺ | |

### Step 3: Preparation of Allyl 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylate

Oxalyl chloride (1.05 g, 8.35 mmol, 5 eq.) was added dropwise to a solution of (R)-((2-((allyloxy)carbonyl)benzo[b]thiophen-5-yl)fluoromethyl)phosphonic acid (550 mg, 1.67 mmol, 1 eq.) in dry DCM (10 mL) and DMF (1 drop) at 25° C. The reaction mixture was stirred at 40° C. for an additional 0.5 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (10 mL), then added to a mixture of phenol (146 mg, 1.56 mmol, 1.05 eq.) and TEA (752 mg, 7.45 mmol, 5 eq.) in anhydrous DCM (5 mL) at 0° C. The reaction was stirred for 0.5 h. Then (1-methyl-1H-tetrazol-5-yl)methyl L-alaninate (3, 551 mg, 2.98 mmol, 2 eq.) was added at 0° C. and the resulting mixture was stirred at 20° C. for 1 h under N₂. After completion, the reaction mixture was concentrated under reduced pressure. The residue was purified by Biotage® C18 column chromatography to give allyl 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylate (90.0 mg, 156 μmol, 11%) as a yellow oil. LCMS (ESI): m/z=574 [M+H]⁺.

The following intermediates in Table 81 were prepared using the method described above in step 3 for the preparation of allyl 5-((1R)-fluoro((((S)-1-((1-methyl-H-tetrazol-5-yl)methoxy)-1-oxopropan-2-yl)amino)(phenoxy)phosphoryl)methyl)benzo[b]thiophene-2-carboxylate and utilizing the appropriate starting materials and modifications.

TABLE 81

| Name | Structure | LCMS |
|---|---|---|
| allyl 5-((1R)-fluoro((((S)-1-((4-methyl-4H- 1,2,4-triazol-3-yl)methoxy)-1-oxopropan-2- yl)amino)(phenoxy)phosphoryl)methyl)benzo [b]thiophene-2-carboxylate | 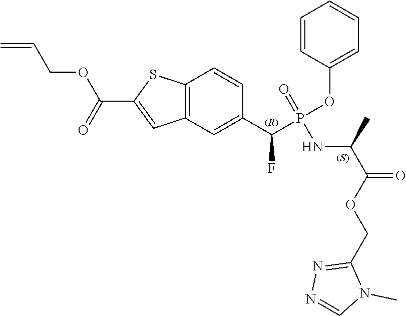 | 573 [M + H]⁺ |
| allyl 5-((1R)-fluoro((((S)-3-methoxy-1-(2- methoxyethoxy)-1-oxopropan-2- yl)amino)(phenoxy)phosphoryl)methyl) benzo[b]thiophene-2-carboxylate | 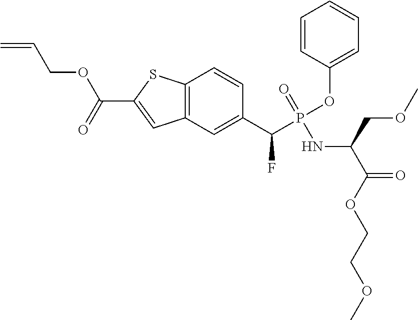 [SMILES: C=CCOC(=O)c1cc2cc([C@H](F)P(=O)(N[C@@H](COC)C(=O)OCCOC)Oc3ccccc3)ccc2s1] | 566 [M + H]⁺ |
| allyl 5-((1R)-fluoro((((S)-1-(2- methoxyethoxy)-1-oxopropan-2- yl)amino)(phenoxy)phosphoryl)methyl) benzo[b]thiophene-2-carboxylate | 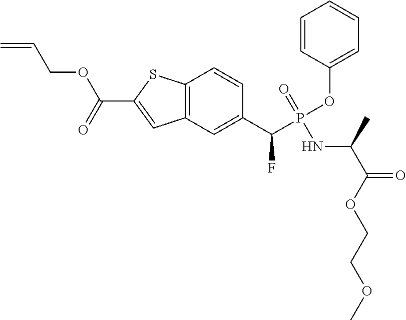 [SMILES: C=CCOC(=O)c1cc2cc([C@H](F)P(=O)(N[C@@H](C)C(=O)OCCOC)Oc3ccccc3)ccc2s1] | 536 [M + H]⁺ |
| allyl 5-((1R)-fluoro((((S)-1-(2- methoxyethoxy)-1-oxopropan-2- yl)amino)(2,2,2- trifluoroethoxy)phosphoryl)methyl) benzo[b]thiophene-2-carboxylate | 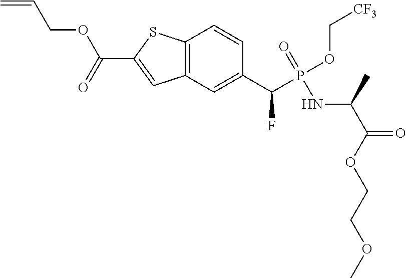 [SMILES: C=CCOC(=O)c1cc2cc([C@H](F)P(=O)(N[C@@H](C)C(=O)OCCOC)OCC(F)(F)F)ccc2s1] | 542 [M + H]⁺ |
| allyl 5-((1R)-fluoro((((S)-1-oxo-1- propoxypropan-2- yl)amino)(phenoxy)phosphoryl)methyl) benzo[b]thiophene-2-carboxylate | 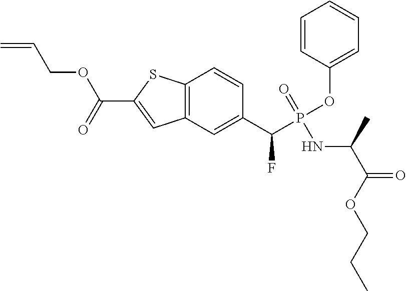 [SMILES: C=CCOC(=O)c1cc2cc([C@H](F)P(=O)(N[C@@H](C)C(=O)OCCC)Oc3ccccc3)ccc2s1] | 520 [M + H]⁺ |
| allyl 5-((1R)-fluoro((((S)-3-methoxy-1-oxo-1- propoxypropan-2- yl)amino)(phenoxy)phosphoryl)methyl) benzo[b]thiophene-2-carboxylate | 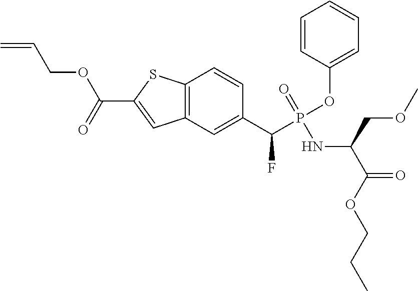 [SMILES: C=CCOC(=O)c1cc2cc([C@H](F)P(=O)(N[C@@H](COC)C(=O)OCCC)Oc3ccccc3)ccc2s1] | 550 [M + H]⁺ |
| allyl 5-((1R)-fluoro((((S)-1-oxo-1-(pyridin-2- ylmethoxy)propan-2- yl)amino)(phenoxy)phosphoryl)methyl) benzo[b]thiophene-2-carboxylate | 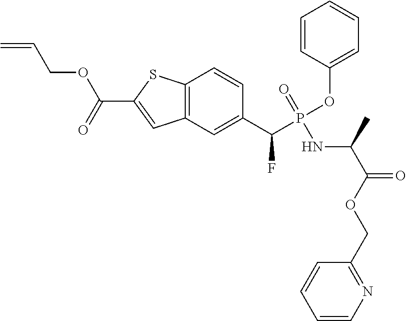 [SMILES: C=CCOC(=O)c1cc2cc([C@H](F)P(=O)(N[C@@H](C)C(=O)OCc3ccccn3)Oc3ccccc3)ccc2s1] | 569 [M + H]⁺ |
| allyl 5-((1R)-fluoro((((S)-1-isobutoxy-1- oxopropan-2- yl)amino)(phenoxy)phosphoryl)methyl) benzo[b]thiophene-2-carboxylate | 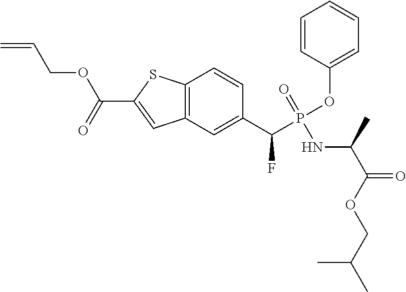 [SMILES: C=CCOC(=O)c1cc2cc([C@H](F)P(=O)(N[C@@H](C)C(=O)OCC(C)C)Oc3ccccc3)ccc2s1] | 534 [M + H]⁺ |
| allyl 5-((1R)-((((S)-1-cyclopropoxy-1- oxopropan-2- yl)amino)(phenoxy)phosphoryl)fluoromethyl) benzo[b]thiophene-2-carboxylate | 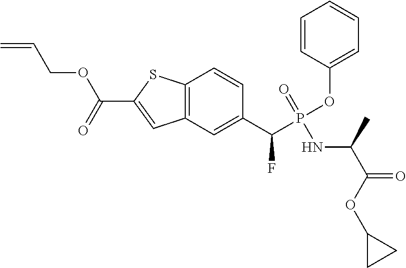 [SMILES: C=CCOC(=O)c1cc2cc([C@H](F)P(=O)(N[C@@H](C)C(=O)OC3CC3)Oc3ccccc3)ccc2s1] | 518 [M + H]⁺ |
| allyl 5-((1R)-((((S)-1-(2-ethylbutoxy)-1- oxopropan-2- yl)amino)(phenoxy)phosphoryl)fluoromethyl) benzo[b]thiophene-2-carboxylate | 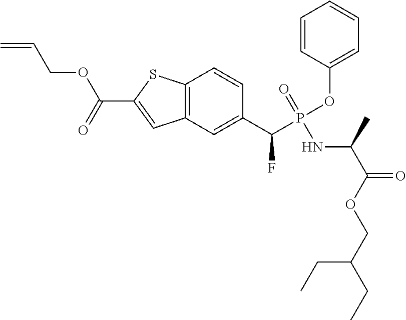 [SMILES: C=CCOC(=O)c1cc2cc([C@H](F)P(=O)(N[C@@H](C)C(=O)OCC(CC)CC)Oc3ccccc3)ccc2s1] | 562 [M + H]⁺ |
| allyl 5-((1R)-((((S)-1-(benzyloxy)-1- oxopropan-2- yl)amino)(phenoxy)phosphoryl)fluoromethyl) benzo[b]thiophene-2-carboxylate | 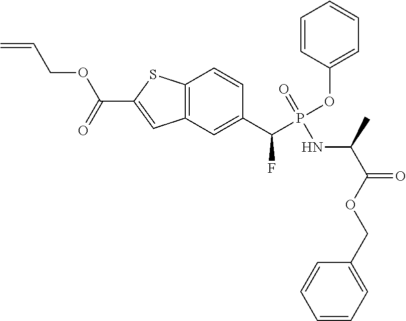 [SMILES: C=CCOC(=O)c1cc2cc([C@H](F)P(=O)(N[C@@H](C)C(=O)OCc3ccccc3)Oc3ccccc3)ccc2s1] | 568 [M + H]⁺ |
| allyl 5-((1R)-fluoro((((S)-1-(neopentyloxy)-1- oxopropan-2- yl)amino)(phenoxy)phosphoryl)methyl) benzo[b]thiophene-2-carboxylate | 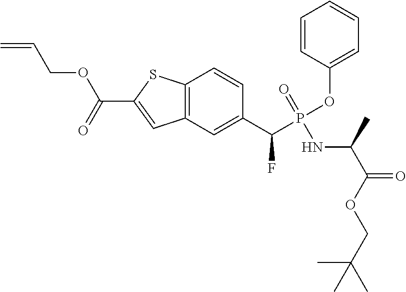 [SMILES: C=CCOC(=O)c1cc2cc([C@H](F)P(=O)(N[C@@H](C)C(=O)OCC(C)(C)C)Oc3ccccc3)ccc2s1] | 548 [M + H]⁺ |
| allyl 5-((1R)-((2,6-dimethylphenoxy)(((S)-1- oxo-1-propoxypropan-2- yl)amino)phosphoryl)fluoromethyl) benzo[b]thiophene-2-carboxylate | 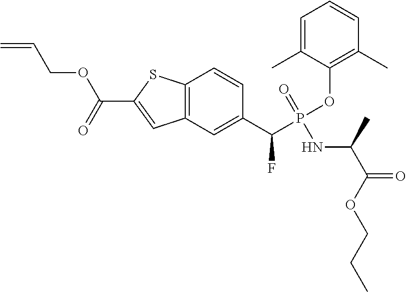 [SMILES: C=CCOC(=O)c1cc2cc([C@H](F)P(=O)(N[C@@H](C)C(=O)OCCC)Oc3c(C)cccc3C)ccc2s1] | 548 [M + H]⁺ |

### Step 4: Preparation of 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylic acid

To a solution of allyl 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylate (90 mg, 156 μmol, 1 eq.) in DCM (5 mL) were added Pd(PPh₃)₄(9.0 mg, 7.8 μmol, 0.05 eq.) and pyrrolidine (11.0 mg, 156 μmol, 1.0 eq.). The mixture was purged and degassed with N₂ (three times), then stirred at 25° C. for 1 hr. After completion, the mixture was concentrated under reduced pressure. The residue was purified by Biotage® C18 column chromatography to give 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylic acid (50.0 mg, 93.7 μmol, 60%) as a yellow oil. LCMS (ESI): m/z=534 [M+H]⁺.

The following intermediates in Table 82 were prepared using the method described above in step 4 for the preparation of 5-((1R)-fluoro((((S)-1-((1-methyl-1H-tetrazol-5-yl)methoxy)-1-oxopropan-2-yl)amino) (phenoxy)phosphoryl)methyl)benzo[b]thiophene-2-carboxylic acid and utilizing the appropriate starting materials and modifications.

TABLE 82

| Name | Structure | LCMS |
|---|---|---|
| 5-((1R)-fluoro((((S)-1-((4-methyl-4H-1,2,4- triazol-3-yl)methoxy)-1-oxopropan-2- yl)amino)(phenoxy)phosphoryl)methyl) benzo[b]thiophene-2-carboxylic acid | 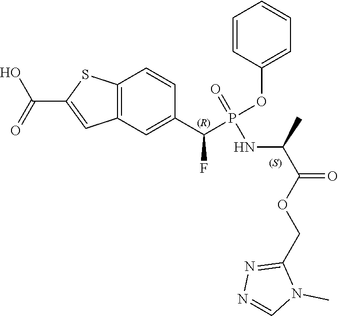 | 533 [M + H]⁺ |
| 5-((1R)-fluoro((((S)-3-methoxy-1-(2- methoxyethoxy)-1-oxopropan-2- yl)amino)(phenoxy)phosphoryl)methyl) benzo[b]thiophene-2-carboxylic acid | 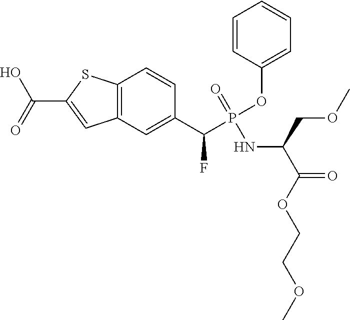 [SMILES: COCCOC(=O)[C@H](COC)NP(=O)(Oc1ccccc1)[C@@H](F)c1ccc2sc(C(=O)O)cc2c1] | 526 [M + H]⁺ |
| 5-((1R)-fluoro((((S)-1-(2-methoxyethoxy)-1- oxopropan-2- yl)amino)(phenoxy)phosphoryl)methyl) benzo[b]thiophene-2-carboxylic acid | 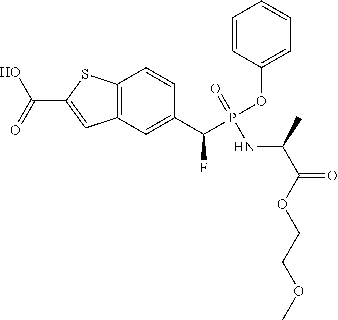 [SMILES: COCCOC(=O)[C@H](C)NP(=O)(Oc1ccccc1)[C@@H](F)c1ccc2sc(C(=O)O)cc2c1] | 496 [M + H]⁺ |
| 5-((1R)-fluoro((((S)-1-(2-methoxyethoxy)-1- oxopropan-2-yl)amino)(2,2,2- trifluoroethoxy)phosphoryl)methyl) benzo[b]thiophene-2-carboxylic acid | 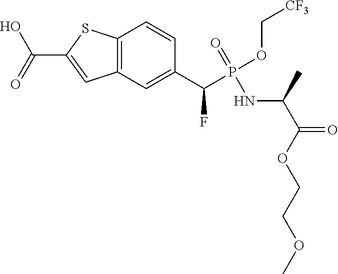 [SMILES: COCCOC(=O)[C@H](C)NP(=O)(OCC(F)(F)F)[C@@H](F)c1ccc2sc(C(=O)O)cc2c1] | 502 [M + H]⁺ |
| 5-((1R)-fluoro((((S)-1-oxo-1-propoxypropan- 2- yl)amino)(phenoxy)phosphoryl)methyl)benzo [b]thiophene-2-carboxylic acid | 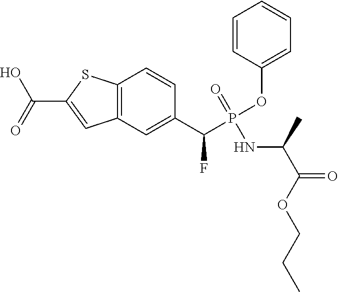 [SMILES: CCCOC(=O)[C@H](C)NP(=O)(Oc1ccccc1)[C@@H](F)c1ccc2sc(C(=O)O)cc2c1] | 480 [M + H]⁺ |
| 5-((1R)-fluoro((((S)-3-methoxy-1-oxo-1- propoxypropan-2- yl)amino)(phenoxy)phosphoryl)methyl) benzo[b]thiophene-2-carboxylic acid | 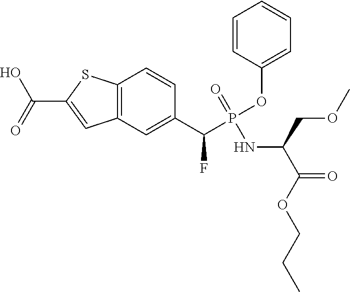 [SMILES: CCCOC(=O)[C@H](COC)NP(=O)(Oc1ccccc1)[C@@H](F)c1ccc2sc(C(=O)O)cc2c1] | 510 [M + H]⁺ |
| 5-((1R)-fluoro((((S)-1-oxo-1-(pyridin-2- ylmethoxy)propan-2- yl)amino)(phenoxy)phosphoryl)methyl) benzo[b]thiophene-2-carboxylic acid | 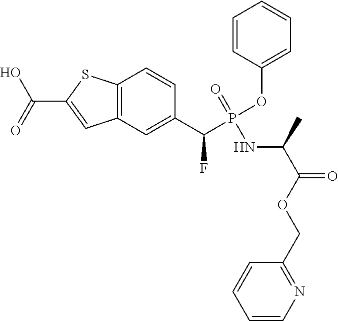 [SMILES: C[C@H](NP(=O)(Oc1ccccc1)[C@@H](F)c1ccc2sc(C(=O)O)cc2c1)C(=O)OCc1ccccn1] | 529 [M + H]⁺ |
| 5-((1R)-fluoro((((S)-1-isobutoxy-1-oxopropan- 2- yl)amino)(phenoxy)phosphoryl)methyl) benzo[b]thiophene-2-carboxylic acid | 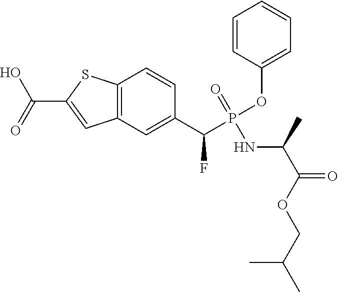 [SMILES: CC(C)COC(=O)[C@H](C)NP(=O)(Oc1ccccc1)[C@@H](F)c1ccc2sc(C(=O)O)cc2c1] | 494 [M + H]⁺ |
| 5-((1R)-((((S)-1-cyclopropoxy-1-oxopropan-2- yl)amino)(phenoxy)phosphoryl)fluoromethyl) benzo[b]thiophene-2-carboxylic acid | 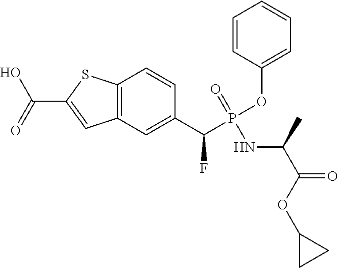 [SMILES: C[C@H](NP(=O)(Oc1ccccc1)[C@@H](F)c1ccc2sc(C(=O)O)cc2c1)C(=O)OC1CC1] | 478 [M + H]⁺ |
| 5-((1R)-((((S)-1-(2-ethylbutoxy)-1-oxopropan- 2- yl)amino)(phenoxy)phosphoryl)fluoromethyl) benzo[b]thiophene-2-carboxylic acid | 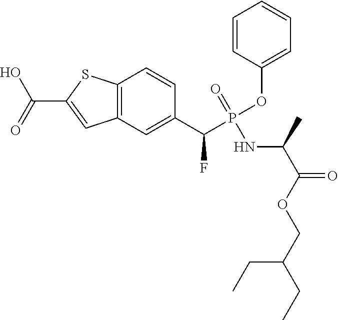 [SMILES: CCC(CC)COC(=O)[C@H](C)NP(=O)(Oc1ccccc1)[C@@H](F)c1ccc2sc(C(=O)O)cc2c1] | 522 [M + H]⁺ |
| 5-((1R)-((((S)-1-(benzyloxy)-1-oxopropan-2- yl)amino)(phenoxy)phosphoryl)fluoromethyl) benzo[b]thiophene-2-carboxylic acid | 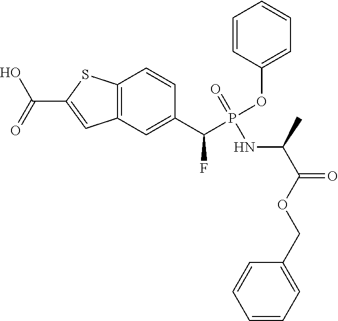 [SMILES: C[C@H](NP(=O)(Oc1ccccc1)[C@@H](F)c1ccc2sc(C(=O)O)cc2c1)C(=O)OCc1ccccc1] | 528 [M + H]⁺. |
| 5-((1R)-((2,6-dimethylphenoxy)(((S)-1-oxo-1- propoxypropan-2- yl)amino)phosphoryl)fluoromethyl) benzo[b]thiophene-2-carboxylic acid | 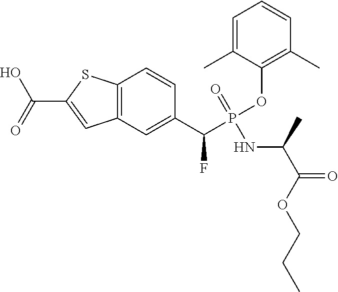 [SMILES: CCCOC(=O)[C@H](C)NP(=O)(Oc1c(C)cccc1C)[C@@H](F)c1ccc2sc(C(=O)O)cc2c1] | 508 [M + H]⁺ |

### Synthesis of 5-((1R)-fluoro((3-(2-methoxyethoxy)phenoxy)(((S)-1-oxo-1-propoxypropan-2-yl)amino)phosphoryl)methyl)benzo[b]thiophene-2-carboxylic acid

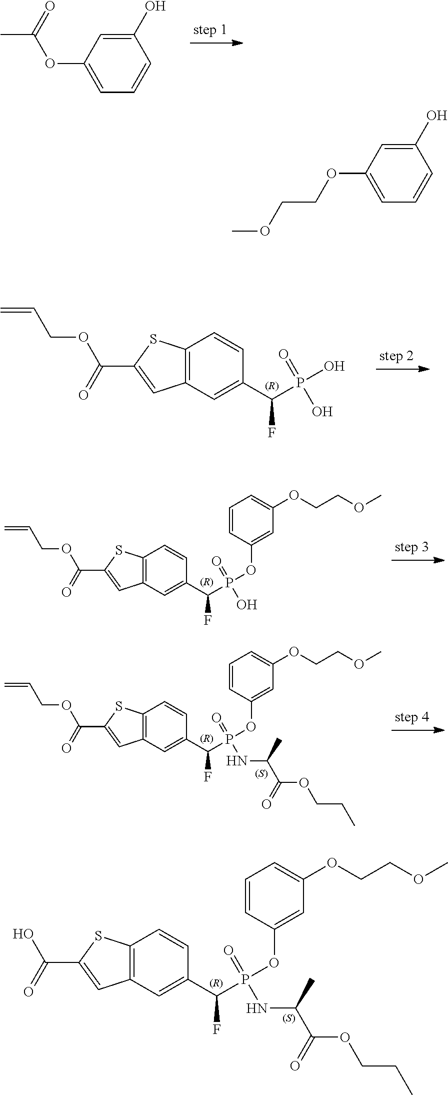

### Step 1: Preparation of 3-(2-methoxyethoxy)phenol

To a solution of 3-hydroxyphenyl acetate (5 g, 32.9 mmol, 1.0 eq.) and K₂CO₃ (13.6 g, 98.7 mmol, 3.0 eq.) in DMF (100 ml) was added 2-methoxyethyl 4-methylbenzenesulfonate (11.4 g, 49.4 mmol, 1.5 eq.). The mixture was stirred at 80° C. for 4 h. After completion, the reaction was cooled to room temperature and 20% NaOH (aq.) was added into the reaction mixture. The resulting mixture was stirred for 0.5 h and then quenched with HCl (1 N, aq.), diluted with H₂O (100 mL) and extracted with DCM (100 mL×3). The organic layers were combined and washed with brine (100 mL), dried over anhydrous Na₂SO₄, and concentrated under reduced pressure. The residue was purified by flash column chromatography on silica gel to afford 3-(2-methoxyethoxy)phenol (4 g, 23.8 mmol, 72%) as a colorless oil. LCMS (ESI): m/z=169 [M+H]⁺.

### Step 2: Preparation of Allyl 5-((IR)-fluoro(hydroxy(3-(2-methoxyethoxy)phenoxy)phosphoryl)methyl)benzo[b]thiophene-2-carboxylate

Oxalyl chloride (3.82 g, 30.3 mmol, 5 eq.) was added dropwise to a solution of (R)-((2-((allyloxy)carbonyl)benzo[b]thiophen-5-yl)fluoromethyl)phosphonic acid (3, 2 g, 6.06 mmol, 1 eq.) in dry DCM (30 mL) and DMF (1 drop) at 25° C. The reaction mixture was stirred at 40° C. for an additional 0.5 h. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (20 mL), then added to a mixture of 3-(2-methoxyethoxy)phenol (1.07 g, 6.36 mmol, 1.05 eq.) and TEA (6.12 g, 60.6 mmol, 10.0 eq.) in anhydrous DCM (10 mL) at 0° C. The reaction was stirred for 0.5 h. After completion, the reaction mixture was concentrated under reduced pressure. The residue was purified by Biotage® C18 column chromatography to give allyl 5-((1R)-fluoro(hydroxy(3-(2-methoxyethoxy)phenoxy)phosphoryl)methyl)benzo[b]thiophene-2-carboxylate (2.00 g, 4.17 mmol, 69%) as a yellow solid. LCMS (ESI): m/z=481 [M+H]⁺.

### Step 3: Preparation of Allyl 5-((1R)-fluoro((3-(2-methoxyethoxy)phenoxy)(((S)-1-oxo-1-propoxypropan-2-yl)amino)phosphoryl)methyl)benzo[b]thiophene-2-carboxylate

Oxalyl chloride (2.63 g, 20.85 mmol, 5 eq.) was added dropwise to the solution of allyl 5-((1R)-fluoro(hydroxy(3-(2-methoxyethoxy)phenoxy)phosphoryl)methyl)benzo[b]thiophene-2-carboxylate (2 g, 4.17 mmol, 1 eq.) in dry DCM (10 mL) and DMF (1 drop) at 25° C. The reaction mixture was stirred at 40° C. for an additional 0.5 h. The reaction was monitored by pipetting out a small amount of crude sample and quenching it with MeOH to ensure mono-Cl phosphoryl chloride had been formed completely (mono-methoxy phosphonate was observed by LCMS). After completion, the excess oxalyl chloride and solvent were removed under reduced pressure. The residue was re-dissolved in anhydrous DCM (20 mL), then added to a mixture of propyl L-alaninate (1.09 g, 8.34 mmol, 2.0 eq.) and TEA (4.21 g, 41.7 mmol, 10.0 eq.) in anhydrous DCM (20 mL) at 0° C. The reaction was stirred for 0.5 h. After completion, the reaction mixture was concentrated under reduced pressure. The residue was purified by Biotage® C₁₈ column chromatography to give allyl 5-((1R)-fluoro((3-(2-methoxyethoxy)phenoxy)(((S)-1-oxo-1-propoxypropan-2-yl)amino)phosphoryl)methyl)benzo[b]thiophene-2-carboxylate (1.8 g, 3.04 mmol, 73%) as a yellow solid. LCMS (ESI): m/z=594 [M+H]⁺.

### Step 4: 5-((1R)-fluoro((3-(2-methoxyethoxy)phenoxy)(((S)-1-oxo-1-propoxypropan-2-yl)amino)phosphoryl)methyl)benzo[b]thiophene-2-carboxylic acid

To a solution of allyl 5-((1R)-fluoro((3-(2-methoxyethoxy)phenoxy)(((S)-1-oxo-1-propoxypropan-2-yl)amino)phosphoryl)methyl)benzo[b]thiophene-2-carboxylate (900 mg, 1.52 mmol, 1.0 eq) in DCM (6 mL) were added Pd(PPh₃)₄(175 mg, 152 μmol, 0.1 eq.) and pyrrolidine (109 mg, 1.52 mmol, 1.0 eq.). The mixture was purged and degassed with N₂ three times, then stirred at 25° C. for 1 hr. After completion, the mixture was concentrated under reduced pressure. The residue was purified by Biotage® C18 column chromatography to give 5-((1R)-fluoro((3-(2-methoxyethoxy)phenoxy)(((S)-1-oxo-1-propoxypropan-2-yl)amino)phosphoryl)methyl)benzo[b]thiophene-2-carboxylic acid (370 mg, 0.67 mmol, 44%) as a white solid. LCMS (ESI): m/z=554 [M+H]⁺.

### Synthesis of perfluorophenyl 5-(fluoro((((S)-1-oxo-1-(3,3,3-trifluoropropoxy)propan-2-yl)amino)(phenoxy)phosphoryl)methyl)benzo[b]thiophene-2-carboxylate

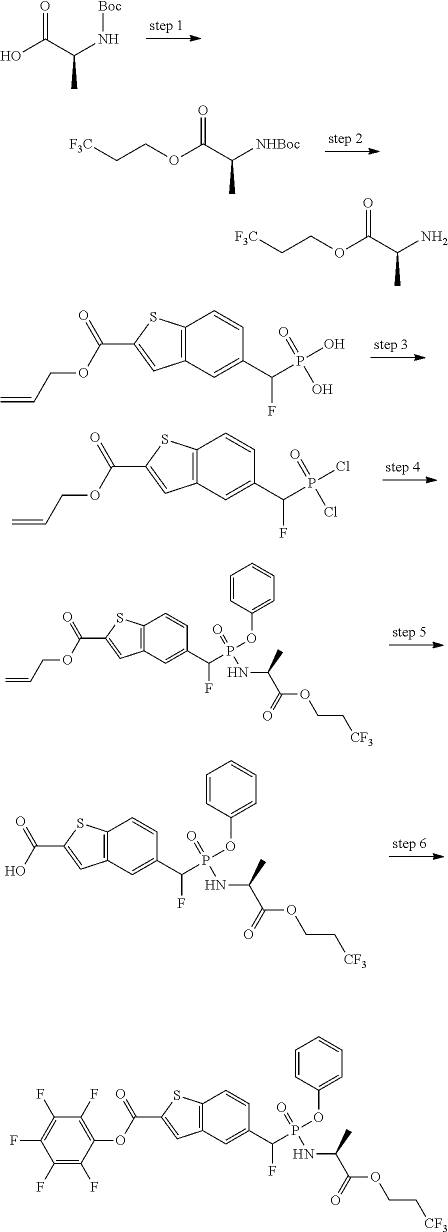

### Step 1: Preparation of (S)-3,3,3-trifluoropropyl 2-((tert-butoxycarbonyl)amino)propanoate

To a solution of (S)-2-((tert-butoxycarbonyl)amino)propanoic acid (5 g, 26.4 mmol, 1 eq) in dimethylformamide (50 mL) was added 3-chloro-1,1,1-trifluoropropane (10 g, 79 mmol, 3 eq) and K₂CO₃ (7.3 g, 53 mmol, 2 eq). The mixture was stirred at 60° C. for 16 h to give a white suspension. The mixture was diluted with water (250 mL) and extracted with EtOAc (1 L×3), the combined organic layers were washed with saturated brine (1 L×3), dried over Na₂SO₄, filtered and concentrated under reduced pressure to give a residue. The residue was purified by flash silica gel chromatography to give a (S)-3,3,3-trifluoropropyl 2-((tert-butoxycarbonyl)amino)propanoate (4.5 g, 15 mmol, 20% yield) as an off-white solid. LCMS (ESI) m/z=286.1 [M+H]⁺.

### Step 2: Preparation of (S)-3,3,3-trifluoropropyl 2-aminopropanoate

The (S)-3,3,3-trifluoropropyl 2-((tert-butoxycarbonyl)amino)propanoate (2 g, 7.0 mmol, 1 eq) was dissolved in DCM (20 mL) and HCL/dioxane (20 mL), the reaction solution was stirred at 25° C. for 1 h. The reaction mixture was concentrated under reduced pressure to give a (S)-3,3,3-trifluoropropyl 2-aminopropanoate (1.1 g, crude) as an off-white solid. ¹H NMR (400 MHz, CD₃OD) δ 4.59-4.40 (m, 2H), 4.15 (q, J=7.2 Hz, 1H), 2.75-2.61 (m, 2H), 1.56 (d, J=7.2 Hz, 3H).

### Step 3: Preparation of Allyl 5-((dichlorophosphoryl)fluoromethyl)benzo[b]thiophene-2-carboxylate

To a solution of ((2-((allyloxy)carbonyl)benzo[b]thiophen-5-yl)fluoromethyl)phosphonic acid (0.90 g, 2.7 mmol, 1 eq) in DCM (9 mL) was added N,N-dimethylformamide (20 mg, 0.27 mmol, 0.1 eq) at 25° C., then cooled to 0° C., then oxalic dichloride (1.2 g, 9.5 mmol, 3.5 eq) was added at 0° C., then stirred at 40° C. for 0.5 h. The reaction mixture was concentrated under reduced pressure to give 5-((dichlorophosphoryl)fluoromethyl)benzo[b]thiophene-2-carboxylate (1.0 g, crude) as a yellow oil. LCMS (ESI) m/z=368.0 [M+H]⁺.

### Step 4: Preparation of Allyl 5-(fluoro((((S)-1-oxo-1-(3,3,3-trifluoropropoxy)propan-2-yl)amino)(phenoxy)phosphoryl)methyl)benzo[b]thiophene-2-carboxylate

To a solution of allyl 5-((dichlorophosphoryl)fluoromethyl)benzo[b]thiophene-2-carboxylate (1.0 g, 2.7 mmol, 1 eq) in DCM (4 mL), the solution was under N₂ and cooled to 0° C., then phenol (0.20 g, 2.2 mmol, 0.8 eq) in DCM (4 mL) was added in it, ethylbis(propan-2-yl)amine (1.4 g, 11 mmol, 4 eq) in DCM (20 mL) was dropwise added in it and stirred at 0° C. for 5 min, (S)-3,3,3-trifluoropropyl 2-aminopropanoate (0.60 g, 3.3 mmol, 1.2 eq) in DCM (4 mL) was added in it and stirred at 0° C. for 10 min to give a yellow solution. The reaction mixture was concentrated under reduced pressure to give a residue. The residue was purified by flash silica gel chromatography to give a product 5-(fluoro((((S)-1-oxo-1-(3,3,3-trifluoropropoxy)propan-2-yl)amino)(phenoxy)phosphoryl)methyl)benzo[b]thiophene-2-carboxylate (0.70 g, 1.2 mmol, 44% yield) as a white solid. LCMS (ESI) m/z=574.1

### Step 5: Preparation of 5-(fluoro((((S)-1-oxo-1-(3,3,3-trifluoropropoxy)propan-2-yl)amino)(phenoxy)phosphoryl)methyl)benzo[b]thiophene-2-carboxylic acid

To a solution of 5-(fluoro((((S)-1-oxo-1-(3,3,3-trifluoropropoxy)propan-2-yl)amino)(phenoxy)phosphoryl)methyl)benzo[b]thiophene-2-carboxylate (0.58 g, 1.0 mmol, 1 eq) and Pd(PPh₃)₄(0.17 g, 0.15 mmol, 1.5 eq) in DCM (5 mL) was added pyrrolidine (50 mg, 0.71 mmol, 0.7 eq) at 0° C. The mixture was stirred at 25° C. for 0.5 h to give a yellow suspension. The reaction mixture was filtrated. The filtered and concentrated under reduced pressure to give a residue. The residue was purified by reverse phase (TFA) to lyophilized to give 5-(fluoro((((S)-1-oxo-1-(3,3,3-trifluoropropoxy)propan-2-yl)amino)(phenoxy)phosphoryl)methyl)benzo[b]thiophene-2-carboxylic acid (0.38 g, 0.71 mmol, 70% yield) as a yellow solid. LCMS (ESI) m/z=534.1 [M+H]⁺.

### Step 6: Preparation of Perfluorophenyl 5-(fluoro((((S)-1-oxo-1-(3,3,3-trifluoropropoxy)propan-2-yl)amino)(phenoxy)phosphoryl)methyl)benzo[b]thiophene-2-carboxylate

To a solution of 5-(fluoro((((S)-1-oxo-1-(3,3,3-trifluoropropoxy)propan-2-yl)amino)(phenoxy)phosphoryl)methyl)benzo[b]thiophene-2-carboxylic acid (0.40 g, 0.75 mmol, 1 eq) in dimethylformamide (4 mL) was added pyridine (0.18 g, 2.2 mmol, 3 eq), then perfluorophenyl 2,2,2-trifluoroacetate (1.0 g, 3.7 mmol, 5 eq) was added in the mixture at 0° C. to give a yellow solution. The mixture was diluted with water (20 mL) and extracted with EtOAc (20 mL×2), the combined organic layers were washed with saturated brine (20 mL×2), dried over Na₂SO₄, filtered and concentrated under reduced pressure to give a residue. The residue was purified by column chromatography to give a product perfluorophenyl 5-(fluoro((((S)-1-oxo-1-(3,3,3-trifluoropropoxy)propan-2-yl)amino)(phenoxy)phosphoryl)methyl)benzo[b]thiophene-2-carboxylate (0.45 mg, 0.64 mmol, 86% yield) as a white solid. LCMS (ESI) m/z=700.0 [M+H]⁺.

### Synthesis of ((6-((perfluorophenoxy)carbonyl)isoquinolin-3-yl)methyl)phosphonic acid

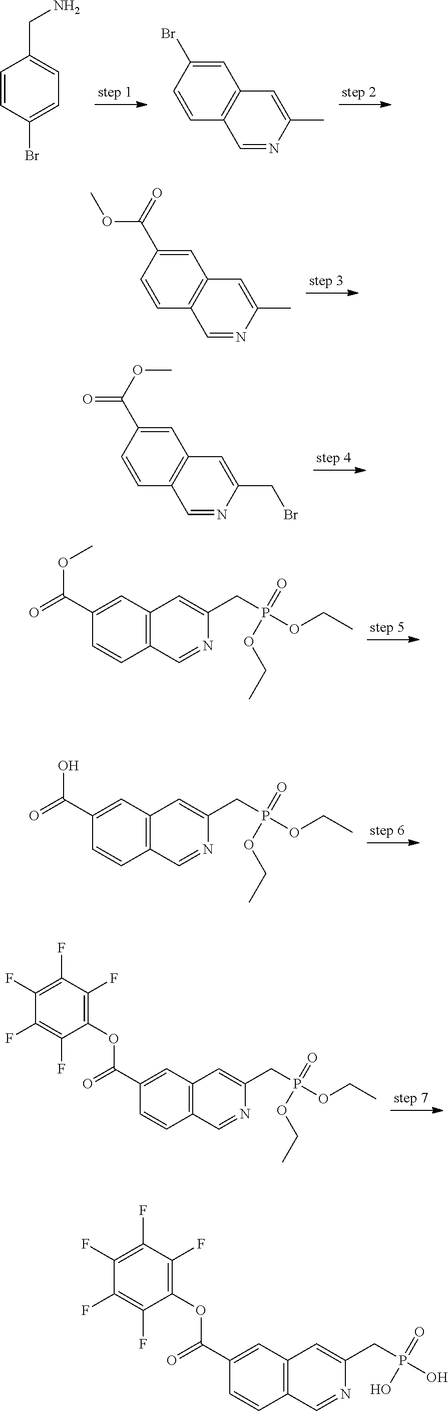

### Step 1: Preparation of 6-bromo-3-methylisoquinoline

To a solution of (4-bromophenyl)methanamine (10 g, 54 mmol, 1 eq) in DCM (5 mL) was added 1,1-dimethoxypropan-2-one (7.0 g, 59 mmol, 1.1 eq) and magnesium (2+) sulfate (10 g, 83 mmol, 1.6 eq), the mixture was stirred at 40° C. for 12 h. Then sodium (boranylidenemethylidene)azanide (3.9 g, 64 mmol, 1.2 eq) was added, the mixture was stirred at 25° C. for 5 h. The mixture was filtered. The filtrate was concentrated to give a yellow oil. Then chlorosulfuric acid (6 mL) was slowly added to the crude product at 0° C. slowly, the reaction mixture was heated to 100° C. for 1 h, then the reaction mixture was cooled to room temperature and poured into ice water. The mixture was neutralized with 2M NaOH and extracted with EtOAc (100 mL×2), the combined organic layers were washed with saturated brine (100 mL×2), dried over Na₂SO₄, filtered and concentrated under reduced pressure to give a residue. The residue was purified by column chromatography to give 6-bromo-3-methylisoquinoline (1.8 g, 8.1 mmol, 15% yield) as a white solid. ¹H NMR (400 MHz, CDCl₃) δ 9.15 (s, 1H), 7.91 (s, 1H), 7.80 (d, J=8.4 Hz, 1H), 7.61 (d, J=8.8 Hz, 1H), 7.39 (s, 1H), 2.71 (s, 3H)

### Step 2: Preparation of Methyl 3-methylisoquinoline-6-carboxylate

To a solution of 6-bromo-3-methylisoquinoline (1.5 g, 6.8 mmol, 1 eq) in methanol (10 mL) was added triethylamine (1.4 g, 14 mmol, 2 eq) and Pd(dppf)Cl₂ (0.5 g, 0.68 mmol, 0.1 eq) under N₂, the mixture was stirred at 70° C. for 16 h under CO (50 psi). The reaction mixture was concentrated under reduced pressure to give a residue. The residue was purified by column chromatography to give methyl 3-methylisoquinoline-6-carboxylate (1.40 g, 7.0 mmol, 95% yield) as a white solid. ¹H NMR (400 MHz, CDCl₃) δ 9.26 (s, 1H), 8.49 (s, 1H), 8.14-8.07 (m, 1H), 8.00 (d, J=8.4 Hz, 1H), 7.59 (s, 1H), 4.01 (s, 3H), 2.74 (s, 3H)

### Step 3: Preparation of Methyl 3-(bromomethyl)isoquinoline-6-carboxylate

To a solution of methyl 3-methylisoquinoline-6-carboxylate (0.40 g, 2.0 mmol, 1 eq) in CCl₄ (6 mL) was added 1-bromopyrrolidine-2,5-dione (35 mg, 0.19 mmol, 0.1 eq) and 2-[(1E)-2-(1-cyano-1-methylethyl)diazen-1-yl]-2-methylpropanenitrile (0.33 g, 2.0 mmol, 1.0 eq), the mixture was stirred at 65° C. for 12 h. The reaction mixture was concentrated under reduced pressure to give a residue. The residue was purified by column chromatography (SiO₂, Petroleum ether/EtOAc=100:1 to 10:1) to give methyl 3-(bromomethyl)isoquinoline-6-carboxylate (0.33 g, 1.2 mmol, 60% yield) as a white solid. ¹H NMR (400 MHz, CDCl₃) δ 9.35 (s, 1H), 8.59 (s, 1H), 8.27-8.20 (m, 1H), 8.12-8.02 (m, 1H), 7.89 (s, 1H), 4.78 (s, 2H), 4.04 (s, 3H)

### Step 4: Preparation of Methyl 3-((diethoxyphosphoryl)methyl)isoquinoline-6-carboxylate

A solution of methyl 3-(bromomethyl)isoquinoline-6-carboxylate (0.3 g, 1.07 mmol, 1 eq) in triethyl phosphite (3 mL) was stirred at 120° C. for 12 h. The reaction mixture was purified by reversed phase (TFA) then lyophilized to give methyl 3-((diethoxyphosphoryl)methyl)isoquinoline-6-carboxylate (0.35 g, 1.04 mmol, 97% yield) as a yellow oil. LCMS (ESI) m/z=338.3.

### Step 5: Preparation of 3-((diethoxyphosphoryl)methyl)isoquinoline-6-carboxylic acid

To a solution of methyl 3-((diethoxyphosphoryl)methyl)isoquinoline-6-carboxylate (0.13 g, 0.39 mmol, 1 eq) in THF (0.5 mL) was added sodium hydroxide (30.70 mg, 770 μmol, 2 eq) in water (0.5 mL), the mixture was stirred at 25° C. for 2 h. The reaction mixture was concentrated under reduced pressure to give 3-((diethoxyphosphoryl)methyl)isoquinoline-6-carboxylic acid (0.13 g, crude) as a yellow solid. LCMS (ESI) m/z=324.0

### Step 6: Preparation of Perfluorophenyl 3-((diethoxyphosphoryl)methyl)isoquinoline-6-carboxylate

To a solution of 3-((diethoxyphosphoryl)methyl)isoquinoline-6-carboxylic acid (0.13 g, 0.40 mol, 1 eq) in DMF (1 mL) was added pyridine (0.13 g, 1.60 mmol, 4 eq), then 2,3,4,5,6-pentafluorophenyl 2,2,2-trifluoroacetate (2.25 g, 8.04 mmol, 20 eq) was added at 0° C., the mixture was stirred at 25° C. for 1 h to give a yellow solution. The mixture was diluted with water (50 mL) and extracted with EtOAc (50 mL×2), the combined organic layers were washed with saturated brine (40 mL×2), dried over Na₂SO₄, filtered and concentrated under reduced pressure to give a residue. The residue was purified by column chromatography to give perfluorophenyl 3-((diethoxyphosphoryl)methyl)isoquinoline-6-carboxylate (80 mg, 0.16 mmol, 40.80% yield) as a yellow solid. LCMS (ESI) m/z=490.2

### Step 7: Preparation of ((6-((perfluorophenoxy)carbonyl)isoquinolin-3-yl)methyl)phosphonic acid

To a solution of perfluorophenyl 3-((diethoxyphosphoryl)methyl)isoquinoline-6-carboxylate (80 mg, 0.16 mmol, 1 eq) in DCM (1 mL) was added iodotrimethylsilane (0.13 g, 0.65 mmol, 4 eq) at 0° C., the mixture was stirred at 0° C. for 10 min. The reaction mixture was concentrated under reduced pressure to remove DCM, then was immediately purified by reversed phase (TFA) to lyophilized to give ((6-((perfluorophenoxy)carbonyl)isoquinolin-3-yl)methyl)phosphonic acid (30.00 mg, 0.07 mmol, 42.40% yield) as a red solid. ¹H NMR (400 MHz, CD₃OD) δ 9.69-9.61 (m, 1H), 9.03 (s, 1H), 8.52-8.47 (m, 1H), 8.46-8.42 (m, 1H), 8.35-8.30 (m, 1H), 3.72-3.59 (m, 2H)

### Synthesis of ((7-((perfluorophenoxy)carbonyl)quinolin-2-yl)methyl)phosphonic acid

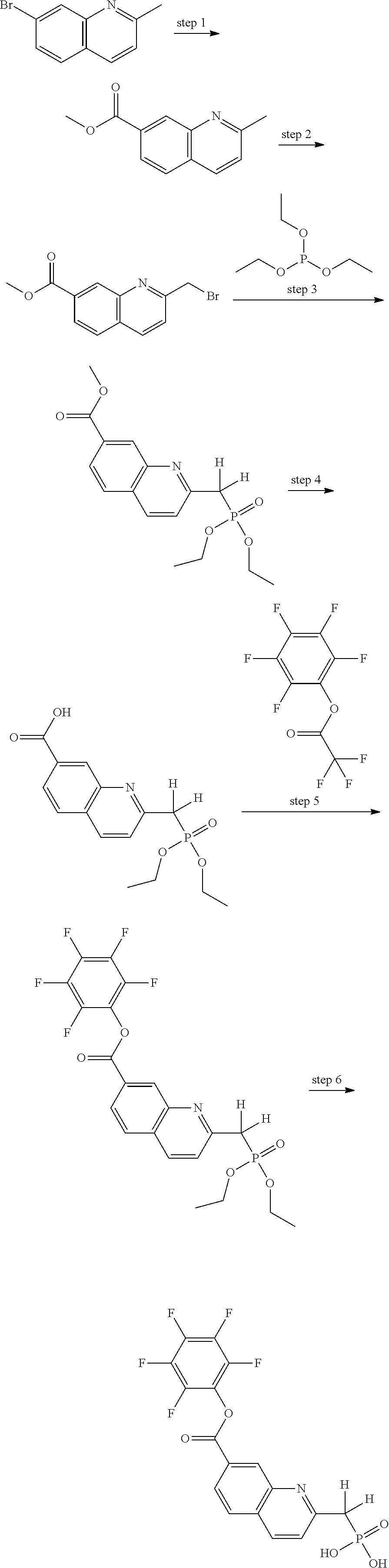

### Step 1: Preparation of Methyl 2-methylquinoline-7-carboxylate

To a solution of 7-bromo-2-methylquinoline (1 g, 4.50 mmol, 1 eq) in MeOH (100 mL) was added TEA (910 mg, 9.00 mmol, 2 eq) and Pd(dppf)Cl₂ (330 mg, 450 μmol, 0.1 eq), and the reaction was stirred for 16 h at 70° C. under CO (50 Psi). The reaction solution was concentrated directly to give a residue. The residue was purified by column chromatography to give methyl 2-methylquinoline-7-carboxylate (850 mg, 4.22 mmol, 93.9% yield) as a white solid. LCMS (ESI) m/z=202.0 [M+H]⁺. ¹H NMR (400 MHz, CD₃OD) δ 8.57 (s, 1H), 8.25 (d, J=8.4 Hz, 1H), 8.04 (d, J=8.4 Hz, 1H), 7.92 (d, J=8.4 Hz, 1H), 7.50 (d, J=8.4 Hz, 1H), 3.98 (s, 3H), 2.73 (s, 3H)

### Step 2: Preparation of Methyl 2-(bromomethyl)quinoline-7-carboxylate

To a solution of methyl 2-methylquinoline-7-carboxylate (850 mg, 4.22 mmol, 1 eq) in chloroform (10 mL) was added NBS (825 mg, 4.64 mmol, 1.1 eq) and AIBN (825 mg, 4.64 mmol, 0.1 eq), the mixture was stirred at 70° C. for 12 h. The mixture was diluted with water (10 mL) and extracted with DCM (20 mL×3). The combined organic layers were washed with brine (20 mL×3), dried over anhydrous Na₂SO₄, filtered and concentrated under reduced pressure to give a residue. The residue was purified by column chromatography to give methyl 2-(bromomethyl)quinoline-7-carboxylate (320 mg, 1.14 mmol, 27.1% yield) as a white solid. LCMS (ESI) m/z=279.8 [M+H]⁺. ¹H NMR (400 MHz, CD₃OD) δ 8.66 (s, 1H), 8.40 (d, J=8.4 Hz, 1H), 8.14 (d, J=8.4 Hz, 1H), 8.01 (d, J=8.4 Hz, 1H), 7.77 (d, J=8.4 Hz, 1H), 4.77 (s, 2H), 4.00 (s, 3H).

### Step 3: Preparation of Methyl 2-((diethoxyphosphoryl)methyl)quinoline-7-carboxylate

A mixture of methyl 2-(bromomethyl)quinoline-7-carboxylate (320 mg, 1.14 mmol, 1 eq) in triethyl phosphite (0.5 mL), the mixture was stirred at 120° C. for 2 h. The mixture was purified by prep-HPLC (column: Welch Xtimate C18 150×25 mm×5 um; mobile phase: [water (TFA)-ACN]; B %: 20%-50%, 10 min) to lyophilized to give methyl 2-((diethoxyphosphoryl)methyl)quinoline-7-carboxylate (350 mg, 1.03 mmol, 91.1% yield) as a white solid. LCMS (ESI) m/z=338.1 [M+H]⁺. ¹H NMR (400 MHz, DMSO-d₆) δ 8.52 (s, 1H), 8.44 (d, J=8.8 Hz, 1H), 8.14-8.02 (m, 2H), 7.65 (d, J=8.4 Hz, 1H), 4.05-3.99 (m, 4H), 3.94 (s, 3H), 3.78-3.63 (m, 2H), 1.19 (t, J=7.2 Hz, 6H)

### Step 4: Preparation of 2-((diethoxyphosphoryl)methyl)quinoline-7-carboxylic acid

To a solution of methyl 2-((diethoxyphosphoryl)methyl)quinoline-7-carboxylate (350 mg, 1.03 mmol, 1 eq) in THF (4 mL) was added and lithium (1+) hydrate hydroxide (86.4 mg, 2.06 mmol, 2 eq, 1 M), the mixture was stirred at 25° C. for 1 h. The mixture was diluted with water (10 mL) and extracted with EtOAc (20 mL×3). The combined aqueous phase was used 1 N HCl to adjust PH=3-4, then extracted with EtOAc (20 mL×3), the combined organic layers were washed with brine (20 mL×3), dried over anhydrous Na₂SO₄, filtered and concentrated under reduced pressure to give 2-((diethoxyphosphoryl)methyl)quinoline-7-carboxylic acid (300 mg, crude) as a white solid. LCMS (ESI) m/z=324.2 [M+H]⁺. ¹H NMR (400 MHz, DMSO-d₆) δ 8.50 (s, 1H), 8.43 (d, J=8.4 Hz, 1H), 8.10-8.02 (m, 2H), 7.68-7.58 (m, 1H), 4.03-3.96 (m, 4H), 3.73-3.66 (m, 2H), 1.19 (t, J=7.2 Hz, 6H)

### Step 5: Preparation of Perfluorophenyl 2-((diethoxyphosphoryl)methyl)quinoline-7-carboxylate

To a solution of 2-((diethoxyphosphoryl)methyl)quinoline-7-carboxylic acid (300 mg, 927 μmol, 1 eq) in DMA (3 mL) was added pyridine (366 mg, 4.63 mmol, 5 eq), then perfluorophenyl 2,2,2-trifluoroacetate (1.29 g, 4.63 mmol, 5 eq) was dropwise at 0° C., the mixture was stirred at 25° C. for 2 h to give a yellow solution. The mixture solution was purified by prep-HPLC (column: Phenomenex Luna C18 200×40 mm×10 um; mobile phase: [water (TFA)-ACN]; B %: 46%-76%, 10 min) to lyophilized to give perfluorophenyl 2-((diethoxyphosphoryl)methyl)quinoline-7-carboxylate (400 mg, 817 μmol, 88.3%) yield as a white solid. ¹H NMR (400 MHz, DMSO-d₆) δ 8.77 (s, 1H), 8.52 (d, J=8.8 Hz, 1H), 8.30-8.10 (m, 2H), 7.73 (d, J=8.4 Hz, 1H), 4.08-4.01 (m, 4H), 3.83-3.63 (m, 2H), 1.36-1.07 (m, 6H)

### Step 6: Preparation of ((7-((perfluorophenoxy)carbonyl)quinolin-2-yl)methyl)phosphonic acid

To a solution of perfluorophenyl 2-((diethoxyphosphoryl)methyl)quinoline-7-carboxylate (200 mg, 408 μmol, 1 eq) in DCM (5 mL) was added iodotrimethylsilane (326 mg, 1.63 mmol, 4 eq), the mixture was stirred at 0° C. for 0.5 h. The mixture was concentrated directly to give the residue. The residue was purified by prep-HPLC (column: Welch Xtimate C18 150×25 mm×5 um; mobile phase: [water (TFA)-ACN]; B %: 20%-50%, 8 min) to lyophilized to give ((7-((perfluorophenoxy)carbonyl) quinolin-2-yl)methyl)phosphonic acid (70 mg, 161 μmol, 39.7% yield) as a yellow solid. LCMS (ESI) m/z=433.7 [M+H]⁺. ¹H NMR (400 MHz, CD₃OD) δ 8.96-8.80 (m, 1H), 8.72-8.58 (m, 1H), 8.38-8.19 (m, 2H), 8.06-7.87 (m, 1H), 3.33 (d, J=1.2 Hz, 2H)

### Synthesis of 6-((diethoxyphosphoryl)methyl)imidazo[1,2-a]pyridine-2-carboxylic acid

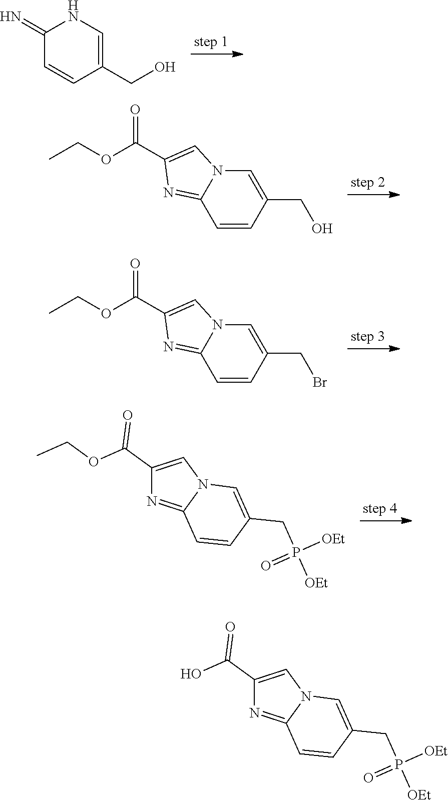

### Step 1: Preparation of Ethyl 6-(hydroxymethyl)imidazo[1,2-a]pyridine-2-carboxylate

To a solution of (6-imino-1,6-dihydropyridin-3-yl)methanol (2 g, 16.1 mmol, 1 eq) in THF (20 mL) was added ethyl 3-bromo-2-oxopropanoate (4.4 g, 22.5 mmol, 1.4 eq) at 25° C., then the mixture was stirred at 80° C. for 12 h to give a yellow suspension. The reaction mixture was concentrated under reduced pressure to give a residue. The residue was purified by flash silica gel chromatography to give ethyl 6-(hydroxymethyl)imidazo[1,2-a]pyridine-2-carboxylate (0.7 g, 2.2 mmol, 18% yield) as a yellow oil. LCMS (ESI) m/z=221.0 [M+H]⁺. ¹H NMR (400 MHz, CD₃OD) δ 8.40 (d, J=16.0 Hz, 2H), 7.56 (d, J=8.0 Hz, 1H), 7.44-7.34 (m, 1H), 4.49-4.32 (m, 2H), 3.35 (s, 2H), 1.40 (t, J=7.2 Hz, 3H)

### Step 2: Preparation of Ethyl 6-(bromomethyl)imidazo[1,2-a]pyridine-2-carboxylate

To a solution of ethyl 6-(hydroxymethyl)imidazo[1,2-a]pyridine-2-carboxylate (0.7 g, 2.2 mmol, 1 eq) in DCM (5 mL) was added triphenylphosphine (0.8 g, 2.9 mmol, 1.3 eq) at 25° C., then the mixture was cooled to 0° C., then a solution of CBr₄ (0.7 g, 2.2 mmol, 1.0 eq) in DCM (1 mL) was slowly dropwise added to the mixture at 0° C., then the mixture was stirred at 25° C. for 1 h to give a yellow solution. The reaction mixture was concentrated under reduced pressure to give a residue. The residue was purified by flash silica gel chromatography to give ethyl 6-(bromomethyl)imidazo[1,2-a]pyridine-2-carboxylate (0.5 g, 1.8 mmol, 80% yield) as a white oil. LCMS (ESI) m/z=284.8 [M+H]⁺.

### Step 3: Preparation of Ethyl 6-((diethoxyphosphoryl)methyl)imidazo[1,2-a]pyridine-2-carboxylate

A solution of ethyl 6-(bromomethyl)imidazo[1,2-a]pyridine-2-carboxylate (0.5 g, 1.8 mmol, 1 eq) in triethyl phosphite (4 mL) was stirred at 100° C. for 12 h to give a white solution. The reaction mixture was purified by reverse phase (TFA) to lyophilized to give ethyl 6-((diethoxyphosphoryl)methyl)imidazo[1,2-a]pyridine-2-carboxylate (0.4 g, 1.0 mmol, 58% yield) as a white oil. LCMS (ESI) m/z=341.3 [M+H]⁺. ¹H NMR (400 MHz, CDCl₃) δ 8.25 (d, J=4.0 Hz, 1H), 8.16 (s, 1H), 8.06 (d, J=12.0 Hz, 1H), 7.45 (d, J=12.0 Hz, 1H), 4.52-4.41 (m, 2H), 4.14-4.10 (m, 4H), 3.25-3.10 (m, 2H), 1.43 (t, J=8.0 Hz, 3H), 1.30 (t, J=8.0 Hz, 6H).

### Step 4: Preparation of 6-((diethoxyphosphoryl)methyl)imidazo[1,2-a]pyridine-2-carboxylic acid

To a solution of ethyl 6-((diethoxyphosphoryl)methyl)imidazo[1,2-a]pyridine-2-carboxylate (0.4 g, 1.0 mmol, 1 eq) in EtOH (0.5 mL) and THF (0.5 mL) was added lithium (1+) hydroxide (5 mL, 2 M), then the mixture was stirred at 25° C. for 2 h to give a white solution. The reaction mixture was concentrated under reduced pressure to give a residue. The residue was purified by reverse phase (TFA) to lyophilized to give 6-((diethoxyphosphoryl)methyl)imidazo[1,2-a]pyridine-2-carboxylic acid (0.2 g, 0.6 mmol, 62.8% yield) as a white oil. LCMS (ESI) m/z=312.9 [M+H]⁺.

### Synthesis of (((2-((perfluorophenoxy)carbonyl)pyrazolo[1,5-a]pyridin-5-yl)methyl)phosphonic acid

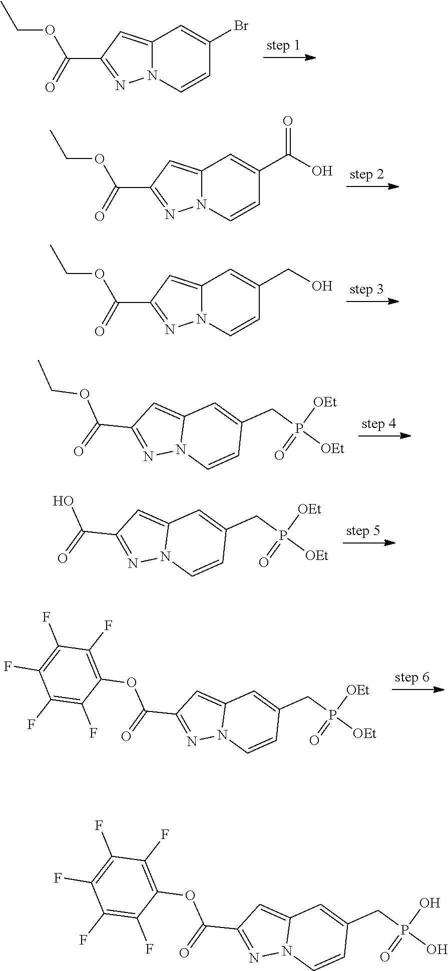

### Step 1: Preparation of 2-(ethoxycarbonyl)pyrazolo[1,5-a]pyridine-5-carboxylic acid

To a solution of ethyl 5-bromopyrazolo[1,5-a]pyridine-2-carboxylate (4 g, 15 mmol, 1 eq) in THF (35 mL) and H₂O (7 mL) was added Pd(DPPF)Cl₂ (2.2 g, 3 mmol, 0.20 eq) and TEA (15 g, 0.15 mol. 10 eq) under CO (45 Psi), the mixture was stirred at 65° C. for 16 h to give black solution. The reaction mixture was filtered and the filter was concentrated to give a residue. The residue was diluted with water (40 mL) and extracted with EtOAc (40 mL×1), the combined organic layers were washed with saturated brine (40 mL×2), dried over Na₂SO₄, filtered and concentrated under reduced pressure to give a residue. The residue was purified by prep-HPLC (TFA condition; (column: Welch Ultimate C18 150×25 mm×5 um, mobile phase: water (TFA)-ACN; B %: B %: 30%-40%, 30 min) to lyophilized to give 2-(ethoxycarbonyl)pyrazolo[1,5-a]pyridine-5-carboxylic acid (1.3 g, 37% yield) as white solid

### Step 2: Preparation of Ethyl 5-(hydroxymethyl)pyrazolo[1,5-a]pyridine-2-carboxylate

To a solution of 2-(ethoxycarbonyl)pyrazolo[1,5-a]pyridine-5-carboxylic acid (1.2 g, 5.1 mmol, 1 eq) in THF (12 mL) was added BH₃.THF (13 mL, 13 mmol, 2.5 eq) at 0° C., the mixture was stirred at 60° C. for 12 h to give a yellow clean solution, the mixture reaction was diluted with water (13 mL×2) at 0° C., and then was extracted with EtOAc (13 mL×2), the combined organic layers were washed with saturated brine (26 mL×2), dried over Na₂SO₄, filtered and concentrated under reduced pressure to give a residue. The residue was purified by flash silica gel chromatography (12 g SepaFlash® Silica Flash Column, Eluent of 0-30% DCM/MeOH ethergradient @12 mL/min) and then was collected and concentrated in vacuo to give ethyl 5-(hydroxymethyl)pyrazolo[1,5-a]pyridine-2-carboxylate (0.40 g, 35% yield) as white solid

### Step 3: Preparation of Ethyl 5-((diethoxyphosphoryl)methyl)pyrazolo[1,5-a]pyridine-2-carboxylate

To a solution of ethyl 5-(hydroxymethyl)pyrazolo[1,5-a]pyridine-2-carboxylate (0.35 g, 1.6 mmol, 1 eq) in triethyl phosphite (2.6 g, 16 mmol, 10 eq) was added tetrabutylammonium ion iodide (0.17 g, 0.47 mmol, 0.30 eq), the mixture was stirred at 120° C. for 16 h to give a yellow clean solution. The reaction was diluted with NaCl (2 mL), and then the reaction was purified by prep-HPLC (TFA condition; (column: Welch Ultimate C18 150×25 mm×5 um, mobile phase: water (TFA)-ACN; B %:B %:30%-50%, 10 min) to lyophilized to give ethyl 5-((diethoxyphosphoryl)methyl)pyrazolo[1,5-a]pyridine-2-carboxylate (0.35 g, 65% yield) as yellow solid. ¹H NMR (400 MHz, CDCl₃) δ 8.46 (d, J=7.2 Hz, 1H), 7.49 (d, J=3.2 Hz, 1H), 7.02 (s, 1H), 6.87 (td, J=1.6, 7.2 Hz, 1H), 4.47 (q, J=7.2 Hz, 2H), 4.13-4.02 (m, 4H), 3.21-3.12 (m, 2H), 1.44 (t, J=7.2 Hz, 3H), 1.27 (t, J=7.2 Hz, 6H)

### Step 4: Preparation of 5-((diethoxyphosphoryl)methyl)pyrazolo[1,5-a]pyridine-2-carboxylic acid

To a solution of 5-((diethoxyphosphoryl)methyl)pyrazolo[1,5-a]pyridine-2-carboxylic acid (0.35 g, 1.0 mmol, 1 eq) in THF (1 mL), MeOH (1 mL), H₂O (1 mL) was added lithium (1+) hydrate hydroxide (43 mg, 1.0 mmol, 1 eq), the mixture was stirred at 25° C. for 0.5 h to give a yellow clean solution, the reaction mixture was concentrated under reduced pressure to give a residue. the residue was diluted with water (5 mL) and extracted with 1 M HCl to pH=5-6, the combined organic layers were washed with EtOAc (5 mL×4), dried over Na₂SO₄, filtered and concentrated under reduced pressure to give 5-((diethoxyphosphoryl)methyl)pyrazolo[1,5-a]pyridine-2-carboxylic acid (0.30 g, 94% yield) as white solid. LCMS (ESI) m/z=313.1

### Step 5: Preparation of Perfluorophenyl 5-((diethoxyphosphoryl)methyl)pyrazolo[1,5-a]pyridine-2-carboxylate

To a solution of 5-((diethoxyphosphoryl)methyl)pyrazolo[1,5-a]pyridine-2-carboxylic acid (0.10 g, 0.32 mmol, 1 eq) in DMF (1 mL) was added pyridine (25 mg, 0.32 mmol, 1 eq), 2,3,4,5,6-pentafluorophenyl 2,2,2-trifluoroacetate (90 mg, 0.32 mmol, 1 eq) at 0° C., the mixture was stirred at 0° C. for 0.5 h to give a yellow clean solution. The reaction was diluted with water (2 mL) and extracted with EtOAc (2 mL×2), the combined organic layers were washed with saturated brine (4 mL×2), dried over Na₂SO₄, filtered and concentrated under reduced pressure to give a residue, the residue was purified by flash silica gel chromatography (4 g SepaFlash® Silica Flash Column, Eluent of 0-30% PE:EA @22 mL/min) and then was collected and concentrated in vacuo to give perfluorophenyl 5-((diethoxyphosphoryl)methyl)pyrazolo[1,5-a]pyridine-2-carboxylate (0.10 g, 65% yield) as a white solid. LCMS (ESI) m/z=479.0

### Step 6: Preparation of ((2-((perfluorophenoxy)carbonyl)pyrazolo[1,5-a]pyridin-5-yl)methyl)phosphonic acid

To a solution of perfluorophenyl 5-((diethoxyphosphoryl)methyl)pyrazolo[1,5-a]pyridine-2-carboxylate (0.10 g, 0.21 mmol, 1 eq) in DCM (1 mL) was added BSTFA (0.16 g, 0.63 mmol, 3 eq), trimethylsilyl iodide (42 mg, 0.21 mmol, 1 eq) at 0° C., the mixture was stirred at 0° C. for 10 min to give a yellow clean solution, the reaction mixture was concentrated under reduced pressure to give a residue, the residue was purified by prep-HPLC (TFA condition; (column: Welch Ultimate C18 150×25 mm×5 um, mobile phase: water (TFA)-ACN; B %:B %: 20%-40%, 10 min) to lyophilized to give ((2-((perfluorophenoxy)carbonyl)pyrazolo[1,5-a]pyridin-5-yl)methyl)phosphonic acid (60 mg, 68% yield) as a white solid. LCMS (ESI) m/z=422.9

### Synthesis of ({6-fluoro-2-[(2,3,4,5,6-pentafluorophenoxy)carbonyl]-1H-indol-5-yl}methyl)phosphonic acid

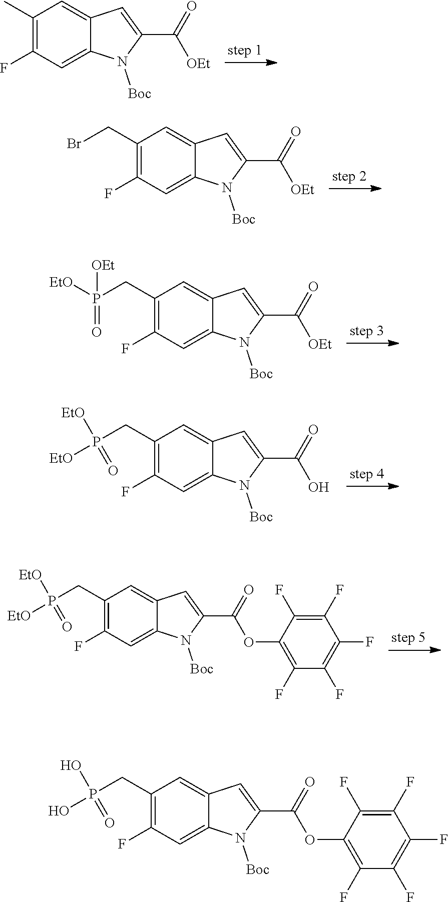

### Step 1: Preparation of 1-(tert-butyl) 2-ethyl 5-(bromomethyl)-6-fluoro-1H-indole-1,2-dicarboxylate

To a solution of 1-(tert-butyl) 2-ethyl 6-fluoro-5-methyl-1H-indole-1,2-dicarboxylate (600 mg, 1.86 mmol, 1 eq.) in CCl₄ (10 mL) were added NBS (331 mg, 1.86 mmol, 1.0 eq.) and BPO (45.0 mg, 186 μmol, 0.1 eq.). The mixture was stirred at 80° C. for 6 h under N₂. After completion, the reaction mixture was concentrated under reduced pressure. The residue was purified by flash column chromatography on silica gel to afford 1-(tert-butyl) 2-ethyl 5-(bromomethyl)-6-fluoro-1H-indole-1,2-dicarboxylate (260 mg, 649 μmol, 35%) as a yellow oil. LCMS (ESI) m/z=400 [M+H]⁺.

### Step 2: Preparation of 1-(tert-butyl) 2-ethyl 5-((diethoxyphosphoryl)methyl)-6-fluoro-1H-indole-1,2-dicarboxylate

1-(tert-butyl) 2-ethyl 5-(bromomethyl)-6-fluoro-1H-indole-1,2-dicarboxylate (260 mg, 649 μmol, 1 eq.) was dissolved in triethyl phosphite (322 mg, 1.94 mmol, 3 eq.). The mixture was stirred at 120° C. for 4 h. After completion, the reaction mixture was concentrated under reduced pressure. The residue was purified by flash column chromatography on silica gel and prep-HPLC to afford 1-(tert-butyl) 2-ethyl 5-((diethoxyphosphoryl)methyl)-6-fluoro-1H-indole-1,2-dicarboxylate (230 mg, 502 μmol, 78%) as a colorless oil. LCMS (ESI) m/z=458 [M+H]⁺.

### Step 3: Preparation of 5-((diethoxyphosphoryl)methyl)-6-fluoro-1H-indole-2-carboxylic acid

To a solution of 1-(tert-butyl) 2-ethyl 5-((diethoxyphosphoryl)methyl)-6-fluoro-1H-indole-1,2-dicarboxylate (230 mg, 502 μmol, 1.0 eq.) in MeOH (3 mL) and H₂O (1 mL) was added LiOH·H₂O (97.8 mg, 2.51 mmol, 5 eq.). The mixture was stirred at 25° C. for 16 h. After completion, the reaction mixture was diluted with water (10 mL) and extracted with EtOAc (10 mL×2). The organic layers were combined and washed with brine (10 mL), dried over anhydrous Na₂SO₄, and concentrated under reduced pressure to afford 1-(tert-butoxycarbonyl)-5-((diethoxyphosphoryl)methyl)-6-fluoro-1H-indole-2-carboxylic acid (120 mg, quant.) as a white solid, which was used in next step directly without further purification.

LCMS (ESI) m/z=430 [M+H]⁺.

### Step 4: Preparation of Perfluorophenyl 5-((diethoxyphosphoryl)methyl)-6-fluoro-1H-indole-2-carboxylate

To a solution of 1-(tert-butoxycarbonyl)-5-((diethoxyphosphoryl)methyl)-6-fluoro-1H-indole-2-carboxylic acid (40 mg, 121 μmol, 1 eq.) in DCM (2 mL) were added DCC (49.8 mg, 242 μmol, 2 eq.), DMAP (11.8 mg, 96.8 μmol, 0.8 eq.) and pentafluorophenol (55.5 mg, 302 μmol, 2.5 eq.). The mixture was stirred at 25° C. for 16 h. After completion, the reaction mixture was concentrated under reduced pressure. The residue was pre-purified by flash column chromatography on silica gel and prep-HPLC to afford perfluorophenyl 5-((diethoxyphosphoryl)methyl)-6-fluoro-1H-indole-2-carboxylate (28.0 mg, 56.5 μmol, 47%) as a white solid. LCMS (ESI): m/z=496 [M+H]⁺.

### Step 5: Preparation of ((6-fluoro-2-((perfluorophenoxy)carbonyl)-1H-indol-5-yl)methyl)phosphonic acid

To a solution perfluorophenyl 5-((diethoxyphosphoryl)methyl)-6-fluoro-1H-indole-2-carboxylate (28 mg, 56.5 μmol, 1 eq.) in DCM (1 mL) was added TMSBr (86.4 mg, 565 μmol, 10 eq.). The mixture was stirred at 25° C. for 2 h. After completion, the reaction mixture was concentrated under reduced pressure. The residue was pre-purified by flash column chromatography on silica gel and prep-HPLC to afford ({6-fluoro-2-[(2,3,4,5,6-pentafluorophenoxy)carbonyl]-1H-indol-5-yl}methyl)phosphonic acid (13.0 mg, 29.5 μmol, 52%) as a white solid. LCMS (ESI): m/z=440 [M+H]⁺.

### Synthesis of ((3-((perfluorophenoxy)carbonyl)quinolin-6-yl)methyl)phosphonic acid

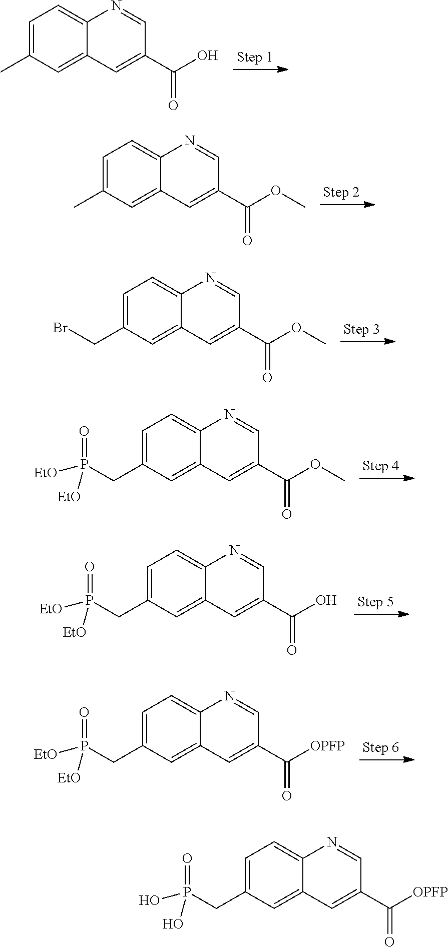

### Step 1: Preparation of Methyl 6-methylquinoline-3-carboxylate

To a solution of 6-methylquinoline-3-carboxylic acid (1, 220 mg, 1.17 mmol, 1.0 eq.) and K₂CO₃ (322 mg, 2.34 mmol, 2.0 eq.) in DMF (10 mL) was added CH₃I (332 mg, 2.34 mmol, 2.0 eq.), and the resulting mixture was stirred at room temperature overnight. After completion, the reaction was diluted with H₂O (10 mL) and extracted with EtOAc (20 mL×3). The organic layers were combined and washed with brine (10 mL), dried over anhydrous Na₂SO₄, and concentrated under reduced pressure. The residue was purified by flash column chromatography on silica gel to afford methyl 6-methylquinoline-3-carboxylate (2, 210 mg, 1.04 mmol, 89%) as a yellow solid. LCMS (ESI): m/z=202.1 [M+H]⁺.

### Step 2: Preparation of Methyl 6-(bromomethyl)quinoline-3-carboxylate

To a solution of methyl 6-methylquinoline-3-carboxylate (2, 210 mg, 1.04 mmol, 1.0 eq.) and NBS (220 mg, 1.24 mmol, 1.2 eq.) in CCl₄ (10 mL) was added BPO (25.1 mg, 0.10 mmol, 0.1 eq.) under N₂, and the resulting mixture was stirred at 70° C. overnight under N₂. After completion, the reaction was cooled to room temperature, diluted with H₂O (10 mL) and extracted with DCM (20 mL×2). The organic layers were combined and washed with brine (10 mL), dried over anhydrous Na₂SO₄, and concentrated under reduced pressure. The residue was purified by flash column chromatography on silica gel to afford methyl 6-(bromomethyl)quinoline-3-carboxylate (3, 225 mg, 803 μmol, 77%) as a white solid. LCMS (ESI): m/z=280.0 [M+H]⁺.

### Step 3: Preparation of Methyl 6-((diethoxyphosphoryl)methyl)quinoline-3-carboxylate

A solution of methyl 6-(bromomethyl)quinoline-3-carboxylate (3, 225 mg, 803 mol, 1.0 eq.) in Triethyl phosphite (5 mL) was stirred at 100° C. overnight. After completion, the reaction was concentrated under reduced pressure. The residue was purified by Biotage® C18 column chromatography to give methyl 6-[(diethoxyphosphoryl)methyl]quinoline-3-carboxylate (4, 220 mg, 652 μmol, 81%) as a colorless oil. LCMS (ESI): m/z=338.1 [M+H]⁺.

¹H NMR (400 MHz, CDCl₃) δ 9.45 (d, J=1.1 Hz, 1H), 8.84 (s, 1H), 8.15 (s, 1H), 7.89 (s, 1H), 7.82 (s, 1H), 4.11-4.05 (m, 4H), 4.04 (s, 3H), 3.38 (d, J=21.9 Hz, 2H), 1.28 (t, J=7.1 Hz, 6H).

### Step 4: Preparation of 6-((diethoxyphosphoryl)methyl)quinoline-3-carboxylic acid

To a solution of methyl 6-[(diethoxyphosphoryl)methyl]quinoline-3-carboxylate (4, 220 mg, 652 μmol, 1 eq.) in THF/H₂O (6 mL, v/v=5:1) was added LiOH—H₂O (84.9 mg, 1.95 mmol, 3.0 eq.), and the resulting mixture was stirred at room temperature for 2 h. After completion, the reaction mixture was cooled down in an ice bath, then neutralized carefully with HCl (2 N, aq.) until the pH was adjusted to pH=3. The resulting mixture was extracted with DCM (10 mL×3), and the combined organic layers were washed with brine (10 mL×2), dried over anhydrous Na₂SO₄, then concentrated under reduced pressure to afford 6-((diethoxyphosphoryl)methyl)quinoline-3-carboxylic acid (5, 180 mg, 556 μmol, 86%) as a yellow solid, which was used in next step directly without further purification. LCMS (ESI): m/z=324.1 [M+H]⁺.

### Step 5: Preparation of Perfluorophenyl 6-((diethoxyphosphoryl)methyl)quinoline-3-carboxylate

Oxalyl chloride (82.3 mg, 649 μmol, 1.5 eq.) was added dropwise to the solution of 6-((diethoxyphosphoryl)methyl)quinoline-3-carboxylic acid (5, 140 mg, 433 μmol, 1.0 eq.) in dry DCM (5 mL) and DMF (2 drops) at 0° C. The reaction mixture was warmed to room temperature, then stirred for 2 h. The reaction was monitored by pipetting out a small amount of crude sample and quenching it with MeOH to ensure acyl chloride had been formed completely. After completion, the excess oxalyl chloride and solvent were removed under reduced pressure. The residue was re-dissolved in anhydrous DCM (5 mL), then added to a mixture of 2,3,4,5,6-pentafluorophenol (143 mg, 779 μmol, 1.8 eq.) and triethylamine (175 mg, 1.73 mmol, 4 eq.) in anhydrous DCM (2 mL) at 0° C. The reaction mixture was allowed to warm to room temperature, and stirred for an additional 30 min. The reaction progress was monitored by LCMS, and after completion, the reaction was quenched by adding H₂O (10 mL) and extracted with DCM (10 mL×3). The organic layers were combined and washed with brine (20 mL), dried over anhydrous Na₂SO₄, and concentrated under reduced pressure. The residue was purified by flash column chromatography on silica gel to afford perfluorophenyl 6-((diethoxyphosphoryl)methyl)quinoline-3-carboxylate (6, 140 mg, 286 μmol, 66%) as a yellow solid. LCMS (ESI): m/z=490 [M+H]⁺.

### Step 6: Preparation of ((3-((perfluorophenoxy)carbonyl)quinolin-6-yl)methyl)phosphonic acid

To a solution of perfluorophenyl 6-((diethoxyphosphoryl)methyl)quinoline-3-carboxylate (6, 140 mg, 286 μmol, 1.0 eq.) in DCM (5 mL) was added TMSBr (1 mL), and the resulting mixture was stirred at room temperature for 16 h. After completion, the reaction mixture was concentrated under reduced pressure to give crude ((3-((perfluorophenoxy)carbonyl)quinolin-6-yl)methyl)phosphonic acid (7, 98.0 mg, 226 μmol, 80%) as a yellow solid, which was used in next step directly without further purification.

LCMS (ESI): m/z=434.1 [M+H]⁺.

### Syntheses of Building Blocks

### Synthesis of 3-(5-fluoro-2-methoxyphenyl)azetidine

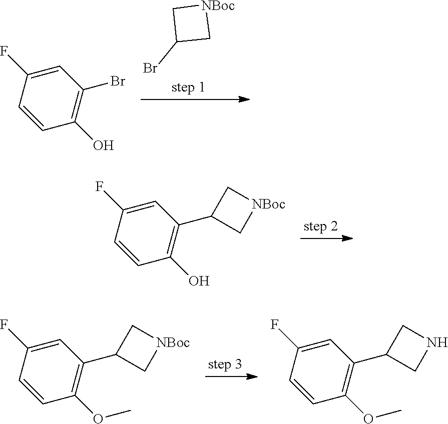

### Step 1: Preparation of Tert-Butyl 3-(5-fluoro-2-hydroxyphenyl)azetidine-1-carboxylate

A reaction vial containing a solution of tert-butyl 3-(5-fluoro-2-hydroxyphenyl)azetidine-1-carboxylate (1 g, 0.2 mmol, 1 eq), tert-butyl 3-bromoazetidine-1-carboxylate (1.24 g, 0.26 mmol, 1.3 eq) Ir[dF(CF₃)ppy]₂(dtbpy)(PF₆) (1.12 g, 0.002 mmol, 0.01 eq), NiCl₂.dtbbpy (595 mg, 0.003 mmol, 0.015 eq), TTMSS (2.48 g, 0.2 mmol, 1 eq), Na₂CO₃ (2.12 g, 0.4 mmol, 2 eq) in DME (10 mL, 1 M) was sealed and placed under an atmosphere of nitrogen. The reaction was stirred and irradiated with a 30 W blue LED lamp (3 cm away), with cooling water to keep the reaction temperature at 25° C. for 14 h. The reaction mixture was diluted with water (100 mL) and extracted with EtOAc (100 mL×2), the combined organic layers were washed with saturated brine (100 mL×2), dried over Na₂SO₄, filtered, and concentrated under reduced pressure to give tert-butyl 3-(5-fluoro-2-hydroxyphenyl)azetidine-1-carboxylate (0.7 g, 67% yield) as a yellow oil.

### Step 2: Preparation of tert-butyl 3-(5-fluoro-2-methoxyphenyl)azetidine-1-carboxylate

To a solution of tert-butyl 3-(5-fluoro-2-hydroxyphenyl)azetidine-1-carboxylate (0.7 g, 2.61 mmol, 1 eq) in acetone (10 mL) was added iodomethane (0.41 g, 2.87 mmol, 1.1 eq) and potassium carbonate (0.54 g, 3.9 mmol, 1.5 eq). The mixture was stirred at 25° C. for 12 h. The reaction was diluted with water (100 mL) and extracted with EtOAc (100 mL×2), the combined organic layers were washed with saturated brine (100 mL×2), dried over Na₂SO₄, filtered and concentrated under reduced pressure to give tert-butyl 3-(5-fluoro-2-methoxyphenyl)azetidine-1-carboxylate (0.3 g, 1.1 mmol, 41% yield) as a yellow oil. LCMS (ESI) m/z=226.3 [M+H]⁺.

### Step 3: Preparation of 3-(5-fluoro-2-methoxyphenyl)azetidine

To a solution of tert-butyl 3-(5-fluoro-2-methoxyphenyl)azetidine-1-carboxylate (0.3 g, 1.06 mmol, 1 eq) in DCM/TFA=3:1 (2 mL). The mixture was stirred at 25° C. for 1 h. The reaction mixture was concentrated under reduced pressure to give 3-(5-fluoro-2-methoxyphenyl)azetidine (0.2 g, crude) as a white solid. LCMS (ESI) m/z=182.4 [M+H]⁺.

### Synthesis of 3-(azetidin-3-yl)-4-fluoro-1-methyl-1H-pyrazole

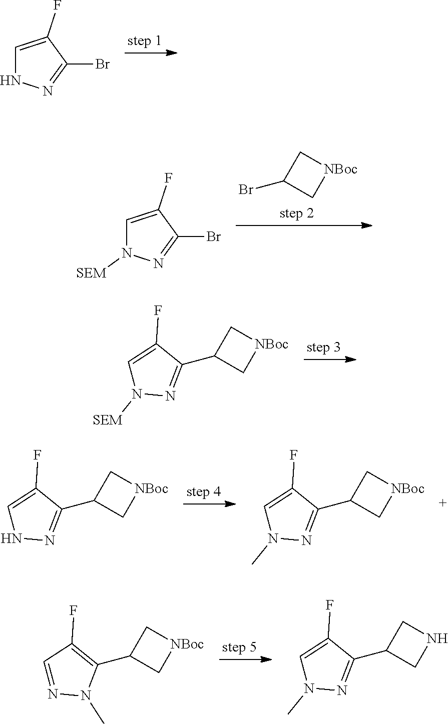

### Step 1: Preparation of 3-bromo-4-fluoro-1-((2-(trimethylsilyl)ethoxy)methyl)-1H-pyrazole

To a solution of 3-bromo-4-fluoro-1H-pyrazole (1.5 g, 9.1 mmol, 1 eq) in THF (15 mL) was added NaH (0.5 g, 13.6 mmol, 1.5 eq) at 0° C. The reaction mixture was stirred at 0° C. for 0.5 h, followed by addition of SEMCl (2.7 g, 9.9 mmol, 1.1 eq) at 0° C., the mixture was stirred at 25° C. for 0.5 h to give a heterogenous solution with white precipitates. The reaction mixture was quenched by water (10 mL) at 0° C. and concentrated under reduced pressure to remove the THF. The mixture was purified by reverse phase (TFA) to lyophilized to give 3-bromo-4-fluoro-1-((2-(trimethylsilyl)ethoxy)methyl)-1H-pyrazole (2.2 g, 7.5 mmol, 82% yield) as a yellow oil. LCMS (ESI) m/z=295.2

### Step 2: Preparation of tert-butyl 3-(4-fluoro-1-((2-(trimethylsilyl)ethoxy)methyl)-1H-pyrazol-3-yl)azetidine-1-carboxylate

To a 15 mL vial equipped with a stir bar was added 3-bromo-4-fluoro-1-((2-(trimethylsilyl)ethoxy)methyl)-1H-pyrazole (2.0 g, 6.8 mmol, 1 eq), tert-butyl 3-bromoazetidine-1-carboxylate (1.9 g, 8.1 mmol, 1.2 eq), Ir[dF(CF₃)ppy]₂(dtbpy)(PF₆) (0.8 g, 0.7 mmol, 0.1 eq), NiCl₂·dtbbpy (0.3 g, 0.7 mmol, 0.1 eq), TTMSS (1.7 g, 6.8 mmol, 1 eq), Na₂CO₃ (1.4 g, 13.5 mmol, 2 eq) and DME (20 mL). The vial was sealed and placed under nitrogen was added. The reaction was stirred and irradiated with a 10 W blue LED lamp (3 cm away), with cooling water to keep the reaction temperature at 25° C. for 14 h to give a brown suspension solution. The reaction mixture was filtered and concentrated under reduced pressure. The residue was purified by reverse phase (TFA) to lyophilized to give tert-butyl 3-(4-fluoro-1-((2-(trimethylsilyl)ethoxy)methyl)-1H-pyrazol-3-yl)azetidine-1-carboxylate (0.8 g, 2.2 mmol, 31.8% yield) as a yellow oil. LCMS (ESI) m/z=315.7 [M+H]⁺. ¹H NMR (400 MHz, CDCl3) δ 7.43-7.33 (m, 1H), 5.34-5.27 (m, 2H), 4.36-4.14 (m, 5H), 3.57-3.43 (m, 2H), 1.47-1.44 (m, 9H), 0.91-0.79 (m, 2H), 0.00-−0.05 (m, 9H).

### Step 3: Preparation of tert-butyl 3-(4-fluoro-1H-pyrazol-3-yl)azetidine-1-carboxylate

To a solution of tert-butyl 3-(4-fluoro-1-((2-(trimethylsilyl)ethoxy)methyl)-1H-pyrazol-3-yl)azetidine-1-carboxylate (0.5 g, 1.5 mmol, 1 eq) in THF (1 mL) was added a solution of TBAF (7.4 mL, 7.5 mmol, 5 eq) in THF at 25° C., then then mixture was stirred at 50° C. for 12 h to give a brown solution. The reaction mixture was concentrated under reduced pressure to give a residue. The residue was purified by reverse phase (TFA) to lyophilized to give tert-butyl 3-(4-fluoro-1H-pyrazol-3-yl)azetidine-1-carboxylate (0.3 g, 1.2 mmol, 84% yield) as a yellow oil. LCMS (ESI) m/z=242.2 [M+H]⁺. ¹H NMR (400 MHz, CDCl₃) δ 7.41 (d, J=4.6 Hz, 1H), 4.34-4.24 (m, 2H), 4.20-4.12 (m, 2H), 3.92-3.77 (m, 1H), 1.45 (s, 9H)

### Step 4: Preparation of tert-butyl 3-(4-fluoro-1-methyl-1H-pyrazol-3-yl)azetidine-1-carboxylate and tert-butyl 3-(4-fluoro-1-methyl-1H-pyrazol-5-yl)azetidine-1-carboxylate

To a solution of tert-butyl 3-(4-fluoro-1H-pyrazol-3-yl)azetidine-1-carboxylate (0.3 g, 1.0 mmol, 1 eq) in THF (3 mL) was added Cs₂CO₃ (0.5 g, 1.5 mmol, 1.5 eq), then cooled to 0° C., then iodomethane (0.3 g, 2.1 mmol, 2 eq) was added to the mixture at 0° C. Then the mixture was stirred at 25° C. for 2 h to give a white suspension. The reaction mixture was filtered and the filtrate was concentrated under reduced pressure to give a residue. The residue was purified by prep-HPLC (column: Welch Xtimate C18 150×25 mm×5 um; mobile phase: water (TFA)-ACN; B %: 32%-52%, 10 min) then lyophilized to give tert-butyl 3-(4-fluoro-1-methyl-1H-pyrazol-3-yl)azetidine-1-carboxylate (80 mg, 0.3 mmol, 30% yield) as a yellow oil. LCMS (ESI) m/z=200.0 [M+H]⁺. ¹H NMR (400 MHz, CDCl3) δ 7.26 (s, 1H), 4.30 (m, 2H), 4.22 (m, 2H), 3.91-3.87 (m, 1H), 3.85 (s, 3H), 1.51 (s, 9H). Additionally, tert-butyl 3-(4-fluoro-1-methyl-1H-pyrazol-5-yl)azetidine-1-carboxylate (40 mg, 0.1 mmol, 15% yield) was isolated as a yellow oil. LCMS (ESI) m/z=256.0 [M+H]⁺. ¹H NMR (400 MHz, CDCl3) δ 7.26 (s, 1H), 4.33-4.25 (m, 2H), 4.24-4.16 (m, 2H), 3.87-3.77 (m, 1H), 3.71 (s, 3H), 1.45 (s, 9H)

### Step 5: Preparation of 3-(azetidin-3-yl)-4-fluoro-1-methyl-1H-pyrazole

To a solution of tert-butyl 3-(4-fluoro-1-methyl-1H-pyrazol-3-yl)azetidine-1-carboxylate (60 mg, 0.2 mmol, 1 eq) in DCM (1 mL) was added TFA (0.3 mL). Then the mixture was stirred at 25° C. for 1 h to give a yellow solution. The reaction mixture was concentrated under reduced pressure to give 3-(azetidin-3-yl)-4-fluoro-1-methyl-1H-pyrazole (60.0 mg) as a yellow oil. LCMS (ESI) m/z=156.1 [M+H]⁺.

### Synthesis of 1-(3-phenylazetidin-3-yl)ethan-1-ol

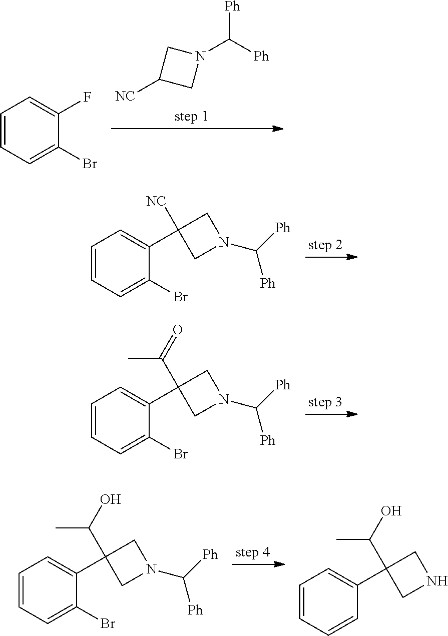

### Step 1: Preparation of 3-(2-bromophenyl)-1-(diphenylmethyl)azetidine-3-carbonitrile

To a cooled (0° C.) solution of 1-bromo-2-fluorobenzene (10 g, 57 mmol, 1.0 eq) and 1-(diphenylmethyl)azetidine-3-carbonitrile (14 g, 57 mmol, 1.0 eq) in THF (120 mL) was added a solution of KHMDS (85 mL, 85 mmol, 1.5 eq) in THF. The mixture was stirred and warmed to 25° C. for 12 h to give a brown solution. The reaction mixture was quenched by H₂O (50 mL) at 0° C. and extracted with EtOAc 150 mL (50 mL×3), dried over Na₂SO₄, filtered and concentrated under reduced pressure to give a residue. The residue was purified by column chromatography to give 3-(2-bromophenyl)-1-(diphenylmethyl)azetidine-3-carbonitrile (5.0 g, 12.4 mmol, 22% yield) as a white solid. ¹H NMR (400 MHz, DMSO-d₆) δ 3.45 (d, J=8.0 Hz, 2H), 3.98 (d, J=8.0 Hz, 2H), 4.55 (s, 1H), 7.23 (d, J=8.0 Hz, 2H), 7.32 (t, J=8.0 Hz, 5H), 7.40-7.49 (m, 6H), 7.69 (d, J=8.0 Hz, 1H).

### Step 2: Preparation of 1-[3-(2-bromophenyl)-1-(diphenylmethyl)azetidin-3-yl]ethan-1-one

To a solution of 3-(2-bromophenyl)-1-(diphenylmethyl)azetidine-3-carbonitrile (4.5 g, 11 mmol, 1.0 eq) in THF (30 mL) was added a solution of methylmagnesium bromide (2.7 g, 22 mol, 2.0 eq) at 0° C. Then mixture was stirred warmed to 40° C. over 12 h. The reaction mixture was quenched by water (100 mL) at 0° C. and extracted with EtOAc (100 mL×3). The combined organic layer was washed with brine (200 mL), dried over Na₂SO₄, filtered and concentrated under reduced pressure to give a residue. The residue was purified by column chromatography to give 1-[3-(2-bromophenyl)-1-(diphenylmethyl)azetidin-3-yl]ethan-1-one (1.8 g, 3.7 mmol, 38% yield) as a white solid. ¹H NMR (400 MHz, CDCl3) δ 7.57 (m, 1H), 7.49 (d, J=8.0 Hz, 4H), 7.31-7.38 (m, 5H), 7.26 (s, 4H), 4.44 (s, 1H), 4.07 (d, J=8.0 Hz, 2H), 3.48 (d, J=8.0 Hz, 2H), 2.38 (s, 3H).

### Step 3: Preparation of 1-[3-(2-bromophenyl)-1-(diphenylmethyl)azetidin-3-yl]ethan-1-ol

To a solution of 1-[3-(2-bromophenyl)-1-(diphenylmethyl)azetidin-3-yl]ethan-1-one (1.8 g, 3.7 mmol, 1.0 eq) in EtOH (20 mL) was added NaBH₄ (828 mg, 22 mmol, 6.0 eq) at 0° C. The mixture was stirred at 25° C. for 4 h to give a suspension. The reaction mixture was quenched by MeOH (50 mL) at 0° C., after stirring 2 h, the mixture was concentrated to give residue. The residue was purified by column chromatography to give 1-[3-(2-bromophenyl)-1-(diphenylmethyl)azetidin-3-yl]ethan-1-ol (1.4 g, 3.3 mmol, 92% yield) as a white solid. LCMS (ESI) m/z=423.9 [M+H]⁺.

### Step 4: Preparation of 1-(3-phenylazetidin-3-yl)ethan-1-ol

To a solution of 1-[3-(2-bromophenyl)-1-(diphenylmethyl)azetidin-3-yl]ethan-1-ol (0.75 g, 1.8 mmol, 1.0 eq) in MeOH (10 mL) was added Pd/C (0.4 g) under N₂, then mixture was stirred at 25° C. for 24 h under H₂ (15 Psi) to give black suspension. The reaction mixture was filtered and concentrated under reduced pressure to give 1-(3-phenylazetidin-3-yl)ethan-1-ol (0.35 g, crude) as a green solid. ¹H NMR (400 MHz, DMSO-d₆) δ 7.37 (d, J=8.0 Hz, 2H) 7.30 (d, J=8.0 Hz, 1H) 7.15-7.20 (m, 2H) 4.29-4.35 (m, 1H) 4.24 (s, 1H) 4.17-4.22 (m, 1H) 3.99 (d, J=12.0 Hz, 1H) 3.86-3.94 (m, 1H) 0.75 (d, J=8.0 Hz, 3H).

### Synthesis of 4-(3-hydroxypyrrolidin-3-yl)pyridin-2(1H)-one

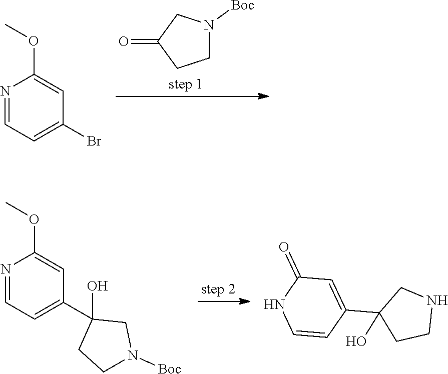

### Step 1: Preparation of Tert-Butyl 3-hydroxy-3-(2-methoxypyridin-4-yl)pyrrolidine-1-carboxylate

To a cooled (−40° C.) solution (under N₂) of 4-bromo-2-methoxypyridine (1.5 g, 8.0 mmol) in THF (10 mL) was added n-BuLi (7.2 mL, 18 mmol). The mixture was stirred at −40° C. for 2 h, followed by addition of tert-butyl 3-oxopyrrolidine-1-carboxylate (1.5 g, 8.0 mmol). The mixture was stirred at −40° C. for 30 min, allowed to warm to 20° C. over 12 h. The resulting orange mixture was quenched by H₂O (50 mL), then was extracted with EtOAc (80 mL×3), the combined organic layers were washed with saturated brine (80 mL×3), dried over Na₂SO₄, filtered and concentrated under reduced pressure to give a residue. The residue was purified by column chromatography to give tert-butyl 3-hydroxy-3-(2-methoxypyridin-4-yl)pyrrolidine-1-carboxylate (0.40 g, 1.36 mmol, 17% yield) as a yellow oil. ¹H NMR (400 MHz, CD₃OD) δ 8.09 (d, J=5.6 Hz, 1H), 7.07 (d, J=5.6 Hz, 1H), 6.96 (s, 1H), 3.91 (s, 3H), 3.64-3.50 (m, 4H), 2.38-2.27 (m, 1H), 2.13-2.05 (m, 1H), 1.48 (d, J=7.6 Hz, 9H).

### Step 2: Preparation of 4-(3-hydroxypyrrolidin-3-yl)pyridin-2(1H)-one

To a solution of tert-butyl 3-hydroxy-3-(2-methoxypyridin-4-yl)pyrrolidine-1-carboxylate (0.1 g, 0.40 mmol) was added HBr/AcOH (3 mL). The mixture was stirred at 80° C. for 12 h to give a yellow solution. The reaction mixture was concentrated under reduced pressure to give 4-(3-hydroxypyrrolidin-3-yl)pyridin-2(1H)-one (0.10 g, crude) as a black oil. LCMS (ESI) m/z=181.0 [M+H]⁺.

### Synthesis of 2-(azetidin-3-yl)-1-methyl-1H-benzo[d]imidazole

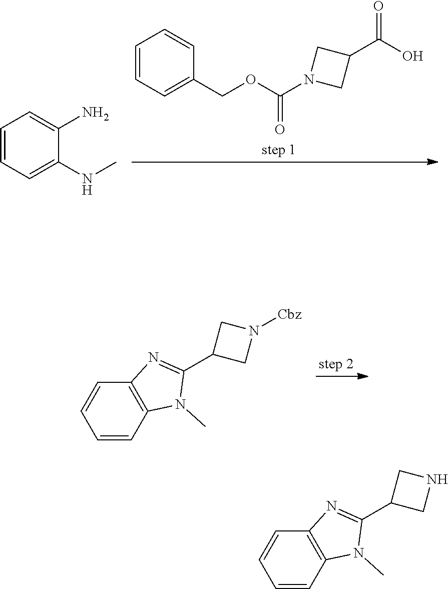

### Step 1: Preparation of Benzyl 3-(1-methyl-1H-benzo[d]imidazol-2-yl)azetidine-1-carboxylate

To a solution of N₁-methylbenzene-1,2-diamine (0.73 g, 6.0 mmol, 1.4 eq) and 1-[(benzyloxy)carbonyl]azetidine-3-carboxylic acid (1 g, 4.3 mmol, 1 eq) in THF (20 mL) was sequentially added EDCI (0.98 g, 5.1 mmol, 1.2 eq) and N,N-diisopropylethylamine (1.6 g, 13 mmol, 3 eq) and 1-hydroxy-7-azabenzotriazole (0.69 g, 5.1 mmol, 1.2 eq). The mixture was stirred at 25° C. for 18 h. the reaction solution was concentrated. The residue was dissolved in acetic acid (10 mL). After stirring at ambient temperature for 2 h. The reaction mixture was concentrated under reduced pressure to give a residue. The residue was purified by flash silica gel chromatography to give benzyl 3-(1-methyl-1H-benzo[d]imidazol-2-yl)azetidine-1-carboxylate (1.3 g, 4.0 mmol, 70% yield) as a yellow solid. LCMS (ESI) m/z=322.1 [M+H]⁺.

### Step 2: Preparation of 2-(azetidin-3-yl)-1-methyl-1H-benzo[d]imidazole

To a solution of benzyl 3-(1-methyl-1H-1,3-benzodiazol-2-yl)azetidine-1-carboxylate (1.30 g, 4.0 mmol, 1 eq) in MeOH (20 mL) was added Pd/C (0.40 g) under N₂, the mixture was stirred at 25° C. under H₂ (15 psi) for 16 h to give a black mixture. The reaction mixture filtered and the filtrate was concentrated under reduced pressure to give 2-(azetidin-3-yl)-1-methyl-1H-benzo[d]imidazole (0.9 g crude) as a black solid. LCMS (ESI) m/z=188.2 [M+H]⁺.

### Synthesis of (R)-3-amino-N,N-dimethylpyrrolidine-1-carboxamide

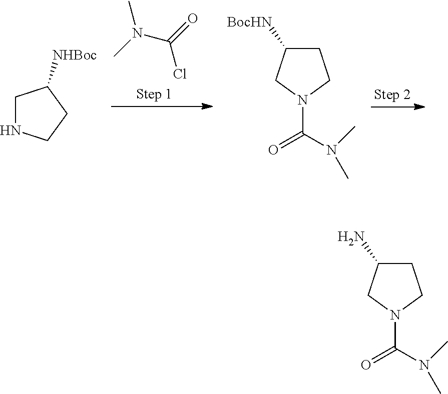

### Step 1: Preparation of (R)-tert-butyl (1-(dimethylcarbamoyl)pyrrolidin-3-yl)carbamate

To a solution of tert-butyl N-[(3R)-pyrrolidin-3-yl]carbamate (300 mg, 1.61 mmol, 1 eq) in methylene chloride (3 mL) was added triethylamine (488 mg, 4.83 mmol, 3 eq) was stirred at 25° C. for 10 min. The mixture was subsequently cooled to 0° C. and dimethylcarbamic chloride (207 mg, 1.93 mmol, 1.2 eq) was dropwise at 0° C. The reaction mixture was stirred at 25° C. for 1 h to give a white suspension. The mixture was diluted with water (10 mL) and extracted with EtOAc (10 mL×2), the combined organic layers were washed with saturated brine (10 mL×2), dried over Na₂SO₄, filtered and concentrated under reduced pressure to give a residue. The residue was purified by flash silica gel chromatography to give (R)-tert-butyl (1-(dimethylcarbamoyl)pyrrolidin-3-yl)carbamate (0.3 g, 0.99 mmol, 62% yield) as a yellow oil. LCMS (ESI) m/z=257.9 [M+H]⁺. ¹H NMR (400 MHz, CDCl3) δ 4.24-4.06 (m, 1H), 3.58 (dd, J=6.0, 10.9 Hz, 1H), 3.46-3.46 (m, 1H), 3.53-3.46 (m, 1H), 3.46-3.37 (m, 1H), 3.24 (d, J=4.0 Hz, 1H), 2.84 (s, 6H), 2.08 (dd, J=5.6, 12.9 Hz, 1H), 1.87-1.73 (m, 1H), 1.45 (s, 9H).

### Step 2: Preparation of (R)-3-amino-N,N-dimethylpyrrolidine-1-carboxamide (2)

To a solution of (R)-tert-butyl (1-(dimethylcarbamoyl)pyrrolidin-3-yl)carbamate (150 mg, 0.58 mmol, 1 eq) in methylene chloride (3 mL) was added TFA (1 mL) and the mixture was stirred at 25° C. for 1 h. The yellow suspension was concentrated under reduced pressure to give (R)-3-amino-N,N-dimethylpyrrolidine-1-carboxamide (150 mg, crude) as a yellow oil. LCMS (ESI) m/z=158.0 [M+H]⁺.

### Synthesis of 3-(azetidin-3-yl)-4-(1,3-dioxolan-2-yl)pyridine

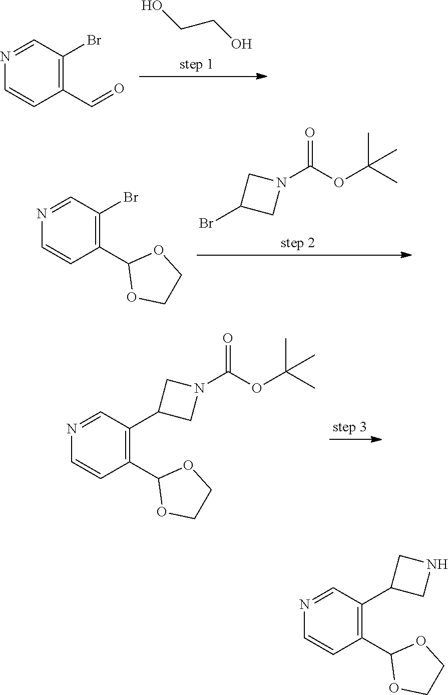

### Step 1: Preparation of 3-bromo-4-(1,3-dioxolan-2-yl)pyridine

To a solution of 3-bromoisonicotinaldehyde (1 g, 5.4 mmol, 1 eq) in toluene (15 mL) was added 4-methylbenzene-1-sulfonic acid (0.28 g, 1.62 mmol, 0.30 eq) and ethane-1,2-diol (0.66 g, 11 mmol, 2 eq). The mixture was stirred at 110° C. for 12 h to give a yellow clean solution. The mixture was purified by flash silica gel chromatography to give 3-bromo-4-(1,3-dioxolan-2-yl)pyridine (0.80 g, 3.48 mmol, 65% yield) as a white solid. ¹H NMR (400 MHz, CDCl3) δ 8.71-8.68 (m, 1H), 8.51 (d, J=4.8 Hz, 1H), 7.45 (d, J=5.2 Hz, 1H), 5.99 (s, 1H), 4.13-4.08 (m, 2H), 4.07-4.03 (m, 2H).

### Step 2: Preparation of tert-butyl 3-(4-(1,3-dioxolan-2-yl)pyridin-3-yl)azetidine-1-carboxylate

To a solution of 3-bromo-4-(1,3-dioxolan-2-yl)pyridine (0.20 g, 0.87 mmol, 1 eq) in DME (5 mL) was added tert-butyl 3-bromoazetidine-1-carboxylate (0.26 g, 1.1 mmol, 1.3 eq), Ir[dF(CF₃)ppy]₂(dtbpy)(PF₆) (11 mg, 8.7 μmol, 0.010 eq), NiCl₂·dtbbpy (5.2 mg, 13 μmol, 0.015 eq), TTMSS (0.22 g, 0.87 mmol, 1 eq) and Na₂CO₃ (0.18 g, 1.7 mmol, 2 eq). The mixture was stirred at 25° C. for 14 h. The resulting homogeneous red solution was concentrated under reduced pressure to give a residue. The residue was diluted with water (20 mL) and extracted with EtOAc (20 mL×2), the combined organic layers were washed with saturated brine (20 mL×2), dried over Na₂SO₄, filtered and concentrated under reduced pressure to give a residue, the residue was purified by flash silica gel chromatography to give tert-butyl 3-(4-(1,3-dioxolan-2-yl)pyridin-3-yl)azetidine-1-carboxylate (100 mg, 0.33 mmol, 37% yield) as yellow oil. LCMS (ESI) m/z=307.1 [M+H]+.

### Step 3: Preparation of 3-(azetidin-3-yl)-4-(1,3-dioxolan-2-yl)pyridine

To a solution of tert-butyl 3-(4-(1,3-dioxolan-2-yl)pyridin-3-yl)azetidine-1-carboxylate (0.10 g, 0.33 mmol, 1 eq) in DCM (1 mL) was added TFA (0.30 mL). The mixture was stirred at 25° C. for 30 min to give a yellow clean solution and the reaction mixture was subsequently concentrated under reduced pressure to give 3-(azetidin-3-yl)-4-(1,3-dioxolan-2-yl)pyridine (0.10 g, crude) as yellow solid.

### Synthesis of 2-(3-methylazetidin-3-yl)pyridine

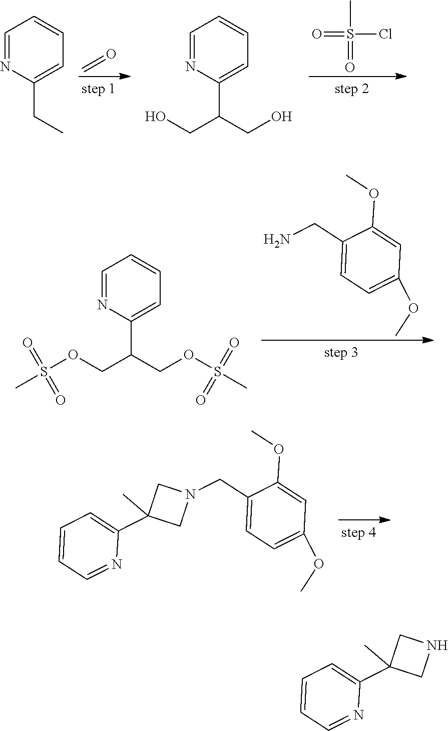

### Step 1: Preparation of 2-methyl-2-(pyridin-2-yl)propane-1,3-diol

A solution of 2-ethylpyridine (25 g, 0.23 mol, 1.0 eq) in formaldehyde (188 mL, 2.3 mol, 10 eq, 37% aqueous solution) was stirred at 140° C. for 68 h under N₂. The mixture was filter and the filtrate was purified by reversed phase (basic system) to lyophilized to give desired 2-methyl-2-(pyridin-2-yl)propane-1,3-diol (0.38 g, 2.3 mmol, 0.98% yield) as a colorless oil. 1H NMR (400 MHz, CD3OD) δ 8.59-8.45 (m, 1H), 7.78 (dt, J=2.0, 8.0 Hz, 1H), 7.55-7.45 (m, 1H), 7.33-7.21 (m, 1H), 3.87-3.83 (m, 4H), 1.33 (s, 3H).

### Step 2: Preparation of 3-(methanesulfonyloxy)-2-methyl-2-(pyridin-2-yl)propylmethanesulfonate

To a cooled (0° C.) solution of 2-methyl-2-(pyridin-2-yl)propane-1,3-diol (0.5 g, 3.0 mmol, 1.0 eq)triethylamine (1.5 g, 15 mmol, 5.0 eq) in DCM (10 mL) was added MsCl (1.4 g, 12 mmol, 4.0 eq) in a dropwise manner. After stirring 1 h, the resulting yellow suspension was quenched by ice water (20 mL) and then was extracted with DCM (30 mL×2). The combined organic layers were washed with saturated NaHCO₃ (30 mL), saturated brine (40 mL×2), dried over Na₂SO₄, filtered and concentrated under reduced pressure to give a residue. The residue was purified by column chromatography to give 3-(methanesulfonyloxy)-2-methyl-2-(pyridin-2-yl)propyl methanesulfonate (0.6 g, crude) as a yellow oil. 1H NMR (400 MHz, CDCl3) δ 8.67-8.46 (m, 1H), 7.73 (dt, J=1.6, 7.6 Hz, 1H), 7.35 (d, J=8.0 Hz, 1H), 7.26-7.20 (m, 1H), 4.67-4.51 (m, 4H), 2.96 (s, 6H), 1.51 (s, 3H).

### Step 3: Preparation of 2-(1-(2,4-dimethoxybenzyl)-3-methylazetidin-3-yl)pyridine

To a solution of 3-(methanesulfonyloxy)-2-methyl-2-(pyridin-2-yl)propyl methanesulfonate (0.42 g, 1.3 mmol, 1 eq) in MeCN (6 mL) was added 1-(2,4-dimethoxyphenyl)methanamine (0.26 g, 1.5 mmol, 1.2 eq) and ethylbis(propan-2-yl)amine (0.5 g, 3.9 mmol, 3.0 eq). The mixture was stirred at 80° C. for 12 h to get a yellow solution. The reaction was concentrated in vacuum to get a residue. The residue was purified by silica gel column to get product 2-(1-(2,4-dimethoxybenzyl)-3-methylazetidin-3-yl)pyridine (0.33 g, 1.1 mmol, 86.1% yield) as a yellow oil. 1H NMR (400 MHz, CDCl3) δ 8.62 (d, J=4.0 Hz, 1H), 7.69 (dt, J=2.0, 7.6 Hz, 1H), 7.36 (d, J=8.4 Hz, 1H), 7.24-7.16 (m, 2H), 6.50 (dd, J=2.4, 8.4 Hz, 1H), 6.45 (d, J=2.4 Hz, 1H), 4.27-4.06 (m, 4H), 3.85-3.79 (m, 6H), 2.84 (s, 2H), 1.80 (s, 3H).

### Step 4: Preparation of 2-(3-methylazetidin-3-yl)pyridine

To a suspension of 2-(1-(2,4-dimethoxybenzyl)-3-methylazetidin-3-yl)pyridine (0.25 g, 0.84 mol, 1.0 eq) in MeCN/H2O (20 mL) was added λ⁴-cerium(4+)bi s(nitric acid) diamine tetranitrate (2.8 g, 5.0 mmol, 6 eq). The mixture was stirred at 60° C. for 4 h to give a yellow solution. The mixture was filter and the filtrate was purified by reversed phase (TFA system) to lyophilized to give 2-(3-methylazetidin-3-yl)pyridine (0.13 g, 0.84 mmol, 90% yield) as a yellow solid. 1H NMR (400 MHz, CD3OD) δ 8.75-8.66 (m, 1H), 8.11 (dt, J=2.0, 8.0 Hz, 1H), 7.66 (d, J=8.0 Hz, 1H), 7.62-7.50 (m, 1H), 4.64 (d, J=11.2 Hz, 2H), 4.21 (d, J=11.2 Hz, 2H), 1.82 (s, 3H).

### Synthesis of 3-(4-fluorothiophen-3-yl)azetidine

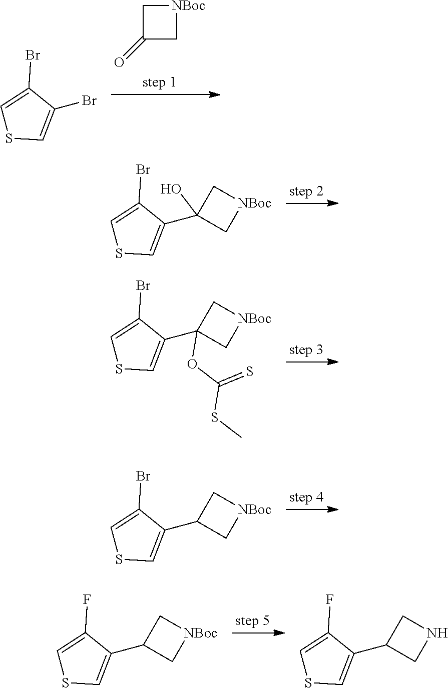

### Step 1: Preparation of Tert-Butyl 3-(4-bromothiophen-3-yl)-3-hydroxyazetidine-1-carboxylate

A solution of 3,4-dibromothiophene (10 g, 41.3 mmol, 1 eq) in THF (100 mL) was cooled it to −78° C. under N₂. To the mixture was slowly added butyllithium (18 mL, 45.4 mmol, 1 eq). The mixture was stirred at −78° C. for 10 min. To the mixture was added tert-butyl 3-oxoazetidine-1-carboxylate (7.77 g, 45.4 mmol, 1.2 eq) solution in THF (100 mL) dropwise over 30 min. The mixture was stirred at −78° C. for 2 h and subsequently warmed to 0° C. The reaction mixture was quenched by saturated NH4Cl (50 mL), then was diluted with water (200 mL) and extracted with EtOAc (200 mL×2). The combined organic layers were washed with saturated brine (200 mL×2), dried over Na₂SO₄, filtered and concentrated under reduced pressure to give a residue. The residue was purified by flash silica gel chromatography to give tert-butyl 3-(4-bromothiophen-3-yl)-3-hydroxyazetidine-1-carboxylate tert-butyl 3-(4-bromothiophen-3-yl)-3-hydroxyazetidine-1-carboxylate (4.40 g, 13.1 mmol, 31.8% yield) as yellow. LCMS (ESI) m/z=357.8 [M+H]+.

### Step 2: Preparation of tert-butyl 3-(4-bromothiophen-3-yl)-3-(((methylthio)carbonothioyl)oxy)azetidine-1-carboxylate

To a cooled (0° C.) solution of tert-butyl 3-(4-bromothiophen-3-yl)-3-hydroxyazetidine-1-carboxylate tert-butyl 3-(4-bromothiophen-3-yl)-3-hydroxyazetidine-1-carboxylate (5 g, 14.9 mmol, 1 eq) in THF (50 mL) under N₂ was added NaH (1.78 g, 44.7 mmol, 3 eq). The mixture stirred at 25° C. for 1.5 h, subsequently cooled to 0° C. and CS2 (2.04 g, 26.8 mmol, 1.8 eq) was slowly added. The mixture was stirred at 0° C. for 1 h, followed was added iodomethane (4.64 g, 32.7 mmol, 2.2 eq) and stirred at 25° C. After stirring for 1.5 h, the reaction mixture was quenched with saturated NH₄Cl (50 mL). The reaction was diluted with water (100 mL) and extracted with DCM (100 mL×2). The combined organic layers were washed with saturated brine (100 mL×2), dried over Na₂SO₄, filtered and concentrated under reduced pressure to give a residue. The residue was purified by flash silica gel chromatography to give tert-butyl 3-(4-bromothiophen-3-yl)-3-(((methylthio)carbonothioyl)oxy)azetidine-1-carboxylate (4.50 g, 10.6 mmol, 71.2% yield) as a white solid.

### Step 3: Preparation of tert-butyl 3-(4-bromothiophen-3-yl)azetidine-1-carboxylate

To a solution of tert-butyl 3-(4-bromothiophen-3-yl)-3-(((methylthio)carbonothioyl)oxy)azetidine-1-carboxylate (1 g, 2.35 mmol, 1 eq) in toluene (10 mL) was added 2-[(1E)-2-(1-cyano-1-methylethyl)diazen-1-yl]-2-methylpropanenitrile (38.5 mg, 235 μmol, 0.1 eq) and tributyltin hydride (1.03 g, 3.52 mmol, 1.5 eq). The mixture was stirred at 25° C. for 12 h. The reaction was diluted with water (100 mL) and extracted with EtOAc (100 mL×2). The combined organic layers were washed with saturated brine (100 mL×2), dried over Na₂SO₄, filtered and concentrated under reduced pressure to give a residue. The residue was purified by column chromatography (SiO2, Petroleum ether/EtOAc=10:1 to 3:1) to give tert-butyl 3-(4-bromothiophen-3-yl)azetidine-1-carboxylate (0.5 g, 1.57 mmol, 66.9% yield) as a white solid. LCMS (ESI) m/z=263.9 [M+H]+.

### Step 4: Preparation of tert-butyl 3-(4-fluorothiophen-3-yl)azetidine-1-carboxylate

To a solution of tert-butyl 3-(4-bromothiophen-3-yl)azetidine-1-carboxylate (0.2 g, 628 μmol, 1 eq) in THF (4 mL) was added N-(benzenesulfonyl)-N-fluorobenzenesulfonamide (472 mg, 1.50 mmol, 2.4 eq) and butyllithium (0.5 mL, 1.38 mmol, 2.2 eq). The mixture was stirred at −78° C. for 3 h. The reaction mixture was quenched by saturated NH₄Cl (50 mL). The reaction was diluted with water (50 mL) and extracted with EtOAc (50 mL×2), the combined organic layers were washed with saturated brine (50 mL×2), dried over Na₂SO₄, filtered, and concentrated under reduced pressure to give a residue. The residue was purified by column chromatography to give tert-butyl 3-(4-fluorothiophen-3-yl)azetidine-1-carboxylate (100 mg, 388 μmol, 62.1% yield) as a white solid. LCMS (ESI) m/z=257.8 [M+H]+.

### Step 5: Preparation of 3-(4-fluorothiophen-3-yl)azetidine

To a solution of tert-butyl 3-(4-fluorothiophen-3-yl)azetidine-1-carboxylate (100 mg, 388 μmol, 1 eq) in DCM/TFA=3:1 (2 mL). The mixture was stirred at 25° C. for 1 h. The reaction mixture was concentrated under reduced pressure to give 3-(4-fluorothiophen-3-yl)azetidine (80 mg, crude) as a yellow oil. LCMS (ESI) m/z=158.1 [M+H]⁺.

### Synthesis of 3-(azetidin-3-yl)-4-fluoro-2-methoxypyridine

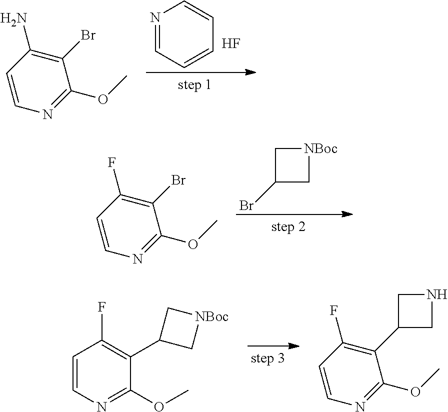

### Step 1: Preparation of 3-bromo-4-fluoro-2-methoxypyridine

A mixture of 3-bromo-2-methoxypyridin-4-amine (500 mg, 2.5 mmol, 1 eq) in pyridine hydrofluoride salt (4 mL) was stirred at −10° C. to the mixture as added sodium nitrite (253 mg, 3.7 mmol, 1.5 eq) in portions (10 batch) to the solution at 0° C. Then the mixture was stirred at 60° C. for 1 h. The reaction mixture was adjusted to pH=7-8 by aqueous solution of 1N Na₂CO₃ at 20° C., then extracted with EtOAc (40 mL×2). Combined organic layers were washed with brine (40 mL×2), dried over anhydrous Na₂SO₄, filtered and concentrated under reduced pressure to give a residue. The residue was purified by column chromatography to give compound 3-bromo-4-fluoro-2-methoxypyridine (300 mg, 1.5 mmol, 59% yield) as a white solid. 1H NMR (400 MHz, CDCl3) δ 8.03 (dd, J=5.6, 7.6 Hz, 1H), 6.72 (dd, J=5.6, 7.2 Hz, 1H), 4.03 (s, 3H).

### Step 2: Preparation of tert-butyl 3-(4-fluoro-2-methoxypyridin-3-yl)azetidine-1-carboxylate

To an 15 mL vial equipped with a stir bar was added 3-bromo-4-fluoro-2-methoxypyridine (41 mg, 0.19 mmol, 1 eq), tert-butyl 3-bromoazetidine-1-carboxylate (61 mg, 0.26 mmol, 1.3 eq), sodium carbonate (42 mg, 0.39 mmol, 2 eq), 1,1,1,3,3,3-hexamethyl-2-(trimethylsilyl)trisilane (49 mg, 0.19 mmol, 1.0 eq), Ir[dF(CF₃)ppy]₂(dtbpy)(PF₆) (2.2 mg, 2.0 μmol, 0.01 eq), NiCl₂·dtbbpy (1.2 mg, 3.0 μmol, 0.02 eq) in DME (2 mL). The vial was sealed and placed under nitrogen was added. The reaction was stirred and irradiated with a 10 W blue LED lamp (3 cm away), with cooling water to keep the reaction temperature at 25° C. for 14 h. The mixture was concentrated under reduced pressure to give a residue. The residue was purified by prep-TLC to give compound tert-butyl 3-(4-fluoro-2-methoxypyridin-3-yl)azetidine-1-carboxylate (20 mg, 71 μmol, 36% yield) as a colorless oil. 1H NMR (400 MHz, CDCl3) δ 7.95-7.91 (m, 1H), 6.60-6.57 (m, 1H), 4.14 (d, J=8.0 Hz, 4H), 3.98-3.96 (m, 1H), 3.92-3.89 (m, 3H), 1.40 (s, 9H)).

### Step 3: Preparation of 3-(azetidin-3-yl)-4-fluoro-2-methoxypyridine

A mixture of tert-butyl 3-(4-fluoro-2-methoxypyridin-3-yl)azetidine-1-carboxylate (20 mg, 71 μmol, 1 eq) in methylene chloride (0.6 mL) and TFA (0.2 mL) was stirred at 20° C. for 0.5 h. The mixture was concentrated under reduced pressure to give compound 3-(azetidin-3-yl)-4-fluoro-2-methoxypyridine (20 mg, crude) as a colorless oil. LCMS (ESI) m/z=183.2 [M+H]⁺.

### Synthesis of 2-methoxy-N-(3-(pyridin-2-yl)azetidin-3-yl)acetamide

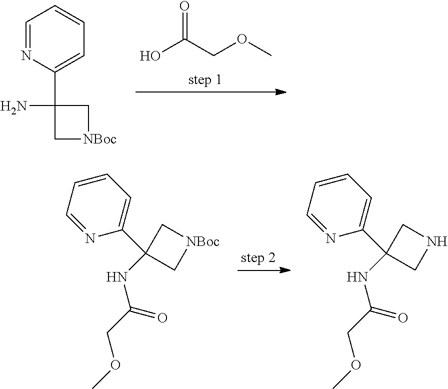

### Step 1: Preparation of Tert-Butyl 3-(2-methoxyacetamido)-3-(pyridin-2-yl)azetidine-1-carboxylate

To a solution of tert-butyl 3-amino-3-(2-pyridyl)azetidine-1-carboxylate (150 mg, 601 μmol, 1 eq), 2-methoxyacetic acid (108 mg, 1.20 mmol, 92 μL, 2 eq) in DMF (2 mL) was added HATU (251 mg, 661 μmol, 1.1 eq) and DIEA (233 mg, 1.80 mmol, 314 μL, 3 eq). The mixture was stirred at 25° C. for 1 h to give a colorless solution. The mixture was diluted with water (10 mL) and extracted with EtOAc (20 mL×3). The combined organic layers were washed with brine (20 mL×3), dried over anhydrous Na₂SO₄, filtered and concentrated under reduced pressure to give a residue. The residue was purified by column chromatography to give tert-butyl 3-[(2-methoxyacetyl)amino]-3-(2-pyridyl)azetidine-1-carboxylate (150 mg, 457 μmol, 76% yield) as an off-white solid. LCMS: (ES+) m/z=322.3 [M+H]+.

### Step 2: Preparation of 2-methoxy-N-(3-(pyridin-2-yl)azetidin-3-yl)acetamide

To a solution of tert-butyl 3-[(2-methoxyacetyl)amino]-3-(2-pyridyl)azetidine-1-carboxylate (150 mg, 466 μmol, 1 eq) in DCM (3 mL) was added TFA (1 mL). The mixture was stirred at 25° C. for 1 h to give a colorless solution. The reaction mixture was concentrated under reduced pressure to give 2-methoxy-N-[3-(2-pyridyl)azetidin-3-yl]acetamide (180 mg, crude) as a colorless oil.

### Synthesis of N-(2-methoxyethyl)-3-(pyridin-2-yl)azetidin-3-amine

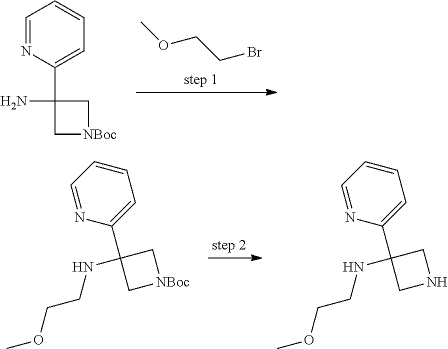

### Step 1: Preparation of Tert-Butyl 3-((2-methoxyethyl)amino)-3-(pyridin-2-yl)azetidine-1-carboxylate

To a solution of tert-butyl 3-amino-3-(2-pyridyl)azetidine-1-carboxylate (150 mg, 601 μmol, 1 eq), 1-bromo-2-methoxy-ethane (167 mg, 1.20 mmol, 113 μL, 2 eq) in CH3CN (2 mL) was added K2CO3 (249 mg, 1.80 mmol, 3 eq) and KI (199 mg, 1.20 mmol, 2 eq). The mixture was stirred at 80° C. for 12 h to give a colorless solution. The mixture was diluted with water (10 mL) and extracted with EtOAc (20 mL×3). The combined organic layers were washed with brine (20 mL×3), dried over anhydrous Na2SO4, filtered and concentrated under reduced pressure to give a residue. The residue was purified by column chromatography to give tert-butyl 3-(2-methoxyethylamino)-3-(2-pyridyl)azetidine-1-carboxylate (100 mg, 286 μmol, 48% yield) as an off-white solid. LCMS (ESI) m/z=308.2 [M+H]+.

### Step 2: Preparation of N-(2-methoxyethyl)-3-(pyridin-2-yl)azetidin-3-amine

To a solution of tert-butyl 3-(2-methoxyethylamino)-3-(2-pyridyl)azetidine-1-carboxylate (100 mg, 286 μmol, 1 eq) in DCM (3 mL) was added TFA (1 mL). The mixture was stirred at 25° C. for 2 h to give a colorless solution. The reaction mixture was concentrated under reduced pressure to give N-(2-methoxyethyl)-3-(pyridin-2-yl)azetidin-3-amine (100 mg, crude) as a colorless oil.

### Synthesis of N-(2,2-difluoroethyl)-3-(pyridin-2-yl)azetidin-3-amine

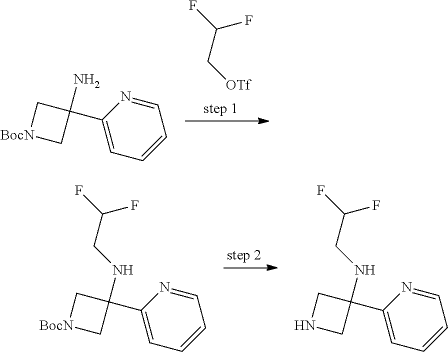

### Step 1: Preparation of Tert-Butyl 3-((2,2-difluoroethyl)amino)-3-(pyridin-2-yl)azetidine-1-carboxylate

To a solution of tert-butyl 3-amino-3-(pyridin-2-yl)azetidine-1-carboxylate (120 mg, 481 μmol, 1 eq), 2,2-difluoroethyl trifluoromethanesulfonate (103 mg, 481 μmol, 1 eq) in CH₃CN (2 mL) was added DIEA (124 mg, 962 μmol, 2 eq). The mixture was stirred at 80° C. for 3 h to give a colorless solution. The mixture was concentrated under reduced pressure to give a residue. The residue was purified by column chromatography to give tert-butyl 3-((2,2-difluoroethyl)amino)-3-(pyridin-2-yl)azetidine-1-carboxylate (140 mg, 447 mol, 92% yield) as an off-white oil. LCMS (ESI) m/z=314.2 [M+H]+.

### Step 2: Preparation of N-(2,2-difluoroethyl)-3-(pyridin-2-yl)azetidin-3-amine

To a solution of tert-butyl 3-((2,2-difluoroethyl)amino)-3-(pyridin-2-yl)azetidine-1-carboxylate (100 mg, 319 μmol, 1 eq) in DCM (3 mL) was added TFA (1 mL). The mixture was stirred at 25° C. for 2 h to give a colorless solution. The reaction mixture was concentrated under reduced pressure to give N-(2,2-difluoroethyl)-3-(pyridin-2-yl)azetidin-3-amine (100 mg, crude) as a colorless oil.

### Synthesis of 3-(3-(2,2-difluoroethoxy)azetidin-3-yl)pyridine

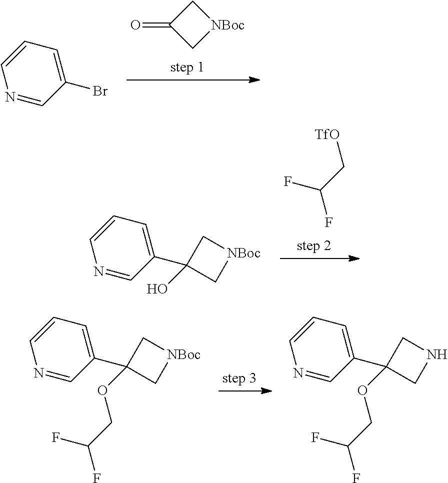 [SMILES: Brc1cccnc1 ; CC(C)(C)OC(=O)N1CC(=O)C1 ; CC(C)(C)OC(=O)N1CC(O)(c2cccnc2)C1 ; O=S(=O)(OCC(F)F)C(F)(F)F ; CC(C)(C)OC(=O)N1CC(OCC(F)F)(c2cccnc2)C1 ; FC(F)COC1(c2cccnc2)CNC1]

### Step 1: Preparation of Tert-Butyl 3-hydroxy-3-(pyridin-3-yl)azetidine-1-carboxylate

To a cooled (−25° C.) solution of 3-bromopyridine (0.5 g, 3.16 mmol, 1 eq) in THF (15 mL) was added iPrMgCl·LiCl (0.5 g, 3.47 mmol, 1.1 eq) slowly under N₂. The reaction mixture was stirred at −25° C. for 0.5 h. To the mixture was added tert-butyl 3-oxoazetidine-1-carboxylate (0.54 g, 3.16 mmol, 1 eq) slowly under N₂. The reaction was stirred for 40 min to give a yellow clean solution. The reaction mixture was quenched with saturated NH4Cl (30 mL) and extracted with EtOAc (30 mL×3), the combined organic layers were washed with saturated brine (40 mL×2), dried over Na2SO4, filtered and concentrated under reduced pressure to give a residue. The residue was purified by column chromatography to give tert-butyl 3-hydroxy-3-(pyridin-3-yl)azetidine-1-carboxylate (0.35 g, 1.39 mmol, 44.3% yield) as a yellow oil. LCMS (ESI) m/z=251.21 [M+H]+.

### Step 2: Preparation of tert-butyl 3-(2,2-difluoroethoxy)-3-(pyridin-3-yl)azetidine-1-carboxylate

To a solution of tert-butyl 3-hydroxy-3-(pyridin-3-yl)azetidine-1-carboxylate (0.35 g, 1.39 mmol, 1 eq) and 2,2-difluoroethyl trifluoromethanesulfonate (0.36 g, 1.66 mmol, 1.2 eq) in THF (8 mL), and the mixture was cooled to 0° C., then was added NaH (0.2 g, 8.34 mmol, 6 eq). The reaction mixture stirred at 25° C. for 2 h. The reaction mixture quenched with saturated NH4Cl (30 mL) and extracted with EtOAc (30 mL×3). The combined organic layers were washed with saturated brine (40 mL×2), dried over Na2SO4, filtered and concentrated under reduced pressure to give a residue. The residue was purified by column chromatography to give tert-butyl 3-(2,2-difluoroethoxy)-3-(pyridin-3-yl)azetidine-1-carboxylate (0.31 g, 990 μmol, 71.3% yield) as a yellow solid. LCMS (ESI) m/z=315.2 [M+H]+.

### Step 3: Preparation of 3-(3-(2,2-difluoroethoxy)azetidin-3-yl)pyridine

To a solution of tert-butyl 3-(2,2-difluoroethoxy)-3-(pyridin-3-yl)azetidine-1-carboxylate (0.2 g, 636 μmol, 1 eq) in DCM (3 mL) and TFA (1 mL). The mixture stirred at 25° C. for 2 h. The reaction mixture was concentrated under reduced pressure to give 3-(3-(2,2-difluoroethoxy)azetidin-3-yl)pyridine (0.13 g, crude) as a yellow oil. LCMS (ESI) m/z=215.0 [M+H]⁺.

### Synthesis of 4-(3-(azetidin-3-yl)pyridin-4-yl)morpholine

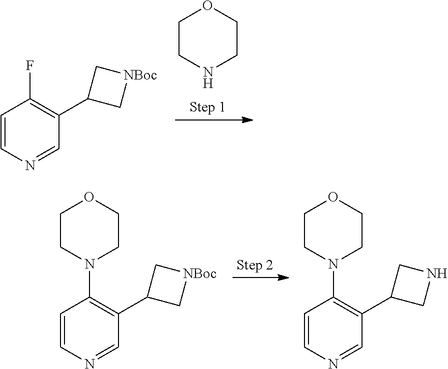 [SMILES: CC(C)(C)OC(=O)N1CC(c2cnccc2F)C1 ; C1COCCN1 ; CC(C)(C)OC(=O)N1CC(c2cnccc2N2CCOCC2)C1 ; c1cc(N2CCOCC2)c(C2CNC2)cn1]

### Step 1: Preparation of Tert-Butyl 3-(4-morpholinopyridin-3-yl)azetidine-1-carboxylate

To a solution of tert-butyl 3-(4-fluoropyridin-3-yl) azetidine-1-carboxylate (0.1 g, 0.4 mmol, 1 eq) in morpholine (1 mL). The mixture was stirred at 160° C. for 5 h under microwave to give a yellow solution. The mixture was purified by flash silica gel chromatography to give tert-butyl 3-(4-morpholinopyridin-3-yl) azetidine-1-carboxylate (0.1 g, 0.28 mmol, 71% yield) as a yellow oil. LCMS (ESI) m/z=320.2 [M+H]+. 1H NMR (400 MHz, CDCl3) δ 8.57-8.53 (m, 1H), 8.48-8.39 (m, 1H), 6.95-6.85 (m, 1H), 4.45-4.30 (m, 2H), 4.11-4.05 (m, 2H), 3.88-3.82 (m, 4H), 3.50 (s, 1H), 2.96-2.86 (m, 4H), 1.48 (s, 9H).

### Step 2: Preparation of 4-(3-(azetidin-3-yl)pyridin-4-yl)morpholine

To a solution of tert-butyl 3-[4-(morpholin-4-yl)pyridin-3-yl]azetidine-1-carboxylate (50 mg, 156 μmol, 1 eq) in DCM (1 mL) was added TFA (0.5 mL). The mixture was stirred at 25° C. for 0.5 h to give a yellow solution. The reaction mixture was concentrated under reduced pressure to give 4-(3-(azetidin-3-yl) pyridin-4-yl) morpholine (50 mg, crude) as a yellow oil. LCMS (ESI) m/z=220.2 [M+H]+.

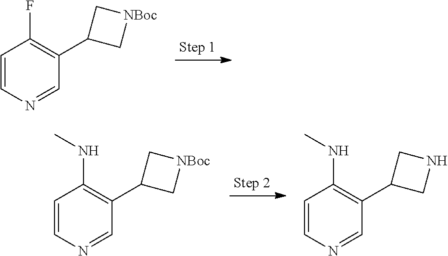

### Synthesis of 3-(azetidin-3-yl)-N-methylpyridin-4-amine carboxylate

To a solution of tert-butyl 3-(4-fluoropyridin-3-yl)azetidine-1-carboxylate (0.19 g, 0.75 mmol, 1 eq), methanamine hydrochloride (0.15 g, 2.3 mmol, 3 eq), DIEA (0.29 g, 2.3 mmol, 3 eq) in DMA (2 mL), the mixture was stirred at 100° C. for 16 h. The mixture solution was diluted with water (10 mL) and extracted with EtOAc (10 mL×3). The combined organic layers were washed with brine (10 mL×3), dried over anhydrous Na2SO4, filtered and concentrated under reduced pressure to give a residue. The residue was purified by flash silica gel chromatography to give tert-butyl 3-[4-(methylamino)p-yridin-3-yl]azetidine-1-carboxylate (0.11 g, 0.42 mmol, 56% yield) as a yellow oil. 1H NMR (400 MHz, DMSO-d6) δ 8.16-8.09 (m, 2H), 6.55 (d, J=6.0 Hz, 1H), 5.75 (s, 2H), 3.87 (t, J=7.4 Hz, 2H), 3.76 (d, J=7.2 Hz, 1H), 2.77 (d, J=4.6 Hz, 3H), 1.38 (s, 9H).

### Step 2: Preparation of 3-(azetidin-3-yl)-N-methylpyridin-4-amine

To a solution of tert-butyl 3-[4-(methylamino)pyridin-3-yl]azetidine-1-carboxylate (80 mg, 0.30 mmol, 1 eq) in DCM (1.5 mL) was added TFA (0.5 mL). After stirring at 25° C. for 1 h, the mixture was concentrated directly to give 3-(azetidin-3-yl)-N-methylpyri-din-4-amine (50 mg, crude) as a yellow oil.

### Synthesis of 3-(azetidin-3-yl)-4-isopropoxypyridine

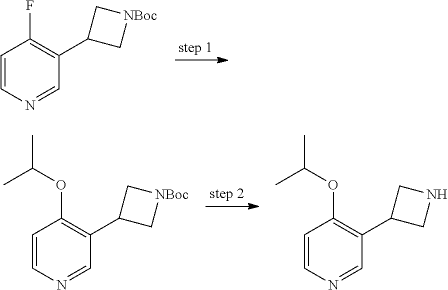

### Step 1: Preparation of Tert-Butyl 3-(4-isopropoxypyridin-3-yl)azetidine-1-carboxylate

To a solution of tert-butyl 3-(4-fluoropyridin-3-yl)azetidine-1-carboxylate (100 mg, 396 μmol, 1 eq) in isopropyl alcohol (1 mL) was added tBuOK (80 mg, 712 μmol, 1.8 eq). The mixture was stirred at 60° C. for 3 h. The mixture was diluted with water (10 mL) and extracted with EtOAc (20 mL×3). The combined organic layers were washed with brine (20 mL×3), dried over anhydrous Na2SO4, filtered and concentrated under reduced pressure to give tert-butyl 3-[4-(propan-2-yloxy)pyridin-3-yl]azetidine-1-carboxylate (90 mg, crude) as a yellow oil. LCMS (ESI) m/z=293.1 [M+H]+.

### Step 2: Preparation of 3-(azetidin-3-yl)-4-isopropoxypyridine

To a solution of tert-butyl 3-[4-(propan-2-yloxy)pyridin-3-yl]azetidine-1-carboxylate (90 mg, 307 μmol, 1 eq) in methylene chloride (1 mL) was added TFA (0.3 mL). The mixture was stirred at 25° C. for 0.5 h. The reaction mixture was concentrated under reduced pressure to give 3-(azetidin-3-yl)-4-(propan-2-yloxy)pyridine (60.0 mg, crude) as a yellow oil. LCMS (ESI) m/z=193.1 [M+H]+.

### Synthesis of (3-(azetidin-3-yl)-4-(oxetan-3-yloxy)pyridine

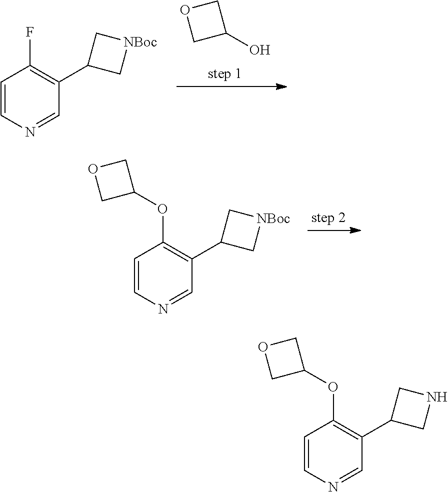 [SMILES: CC(C)(C)OC(=O)N1CC(c2cnccc2F)C1 ; OC1COC1 ; CC(C)(C)OC(=O)N1CC(c2cnccc2OC2COC2)C1 ; c1cc(OC2COC2)c(C2CNC2)cn1]

### Step 1: Preparation of Tert-Butyl 3-(4-(oxetan-3-yloxy)pyridin-3-yl)azetidine-1-carboxylate (1)

To a solution of tert-butyl 3-(4-fluoropyridin-3-yl)azetidine-1-carboxylate (0.12 g, 0.48 mmol, 1 eq) in THF (2 mL) was added NaH (36 mg, 1.4 mmol, 3.0 eq) in portions at 0° C., the reaction mixture was stirred for 30 min, then oxetan-3-ol (35 mg, 0.48 mmol, 1 eq) was added to the mixture. The reaction mixture was slowly heated to 80° C. and stirred for 1 h. The reaction was quenched with water (30 mL) and extracted (30 mL×3), dried over Na2SO4, filtered and concentrated under reduced pressure to give a residue. The organic layer was dried over anhydrous Na2SO4 and concentrated in vacuum to give crude product. The residue was purified by flash silica gel chromatography to give tert-butyl 3-(4-(oxetan-3-yloxy)pyridin-3-yl)azetidine-1-carboxylate (0.13 g, 0.42 mmol, 90% yield) was obtained as a yellow oil. 1H NMR (400 MHz, CDCl3) δ 8.40 (s, 1H), 8.39-8.35 (m, 1H), 6.33 (d, J=5.6 Hz, 1H), 5.30-5.26 (m, 1H), 5.04-4.98 (m, 2H), 4.75-4.71 (m, 2H), 4.36-4.26 (m, 2H), 4.17-4.10 (m, 2H), 4.01-3.90 (m, 1H), 1.49-1.41 (m, 9H)

### Step 2: Preparation of 3-(azetidin-3-yl)-4-(oxetan-3-yloxy)pyridine

To a solution of tert-butyl 3-(4-(oxetan-3-yloxy)pyridin-3-yl)azetidine-1-carboxylate (0.10 g, 0.33 mmol, 1 eq) in DCM (1 mL) was added TFA (0.30 mL). The mixture was stirred at 25° C. for 1 h to give a yellow solution. The reaction mixture was concentrated under reduced pressure to give 3-(azetidin-3-yl)-4-(oxetan-3-yloxy)pyridine (100 mg, crude) as a yellow oil.

### Synthesis of 5-(azetidin-3-yl)-N,N-dimethylpyrimidin-4-amine

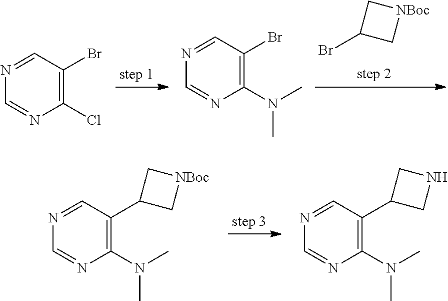

### Step 1: Preparation of 5-bromo-N,N-dimethylpyrimidin-4-amine

To a solution of 5-bromo-4-chloropyrimidine (0.85 g, 4.39 mmol, 1 eq) in THF (4 mL) was added dimethylamine (4 mL, 4.39 mmol, 1.0 eq). The mixture was stirred at 65° C. for 4 h to give a yellow mixture. The mixture was diluted with water (50 mL) and extracted with EtOAc (50 mL×2), the combined organic layers were washed with saturated brine (40 mL×2), dried over Na2SO4, filtered, and concentrated under reduced pressure to give 5-bromo-N,N-dimethylpyrimidin-4-amine (735 mg, 3.64 mmol, 83% yield) as a yellow oil. LCMS (ESI) m/z=204.1 [M+H]+. 1H NMR (400 MHz, CD3OD) 8.38 (s, 1H), 8.29 (s, 1H), 3.24 (s, 6H).

### Step 2: Preparation of tert-butyl 3-(4-(dimethylamino)pyrimidin-5-yl)azetidine-1-carboxylate

To a 15 mL vial equipped with a stir bar was added tert-butyl 3-bromoazetidine-1-carboxylate (0.11 g, 0.49 mmol, 1 eq), 5-bromo-N,N-dimethylpyrimidin-4-amine (0.10 g, 0.49 mmol, 1 eq), Ir[dF(CF₃)ppy]₂(dtbpy)(PF₆) (2.24 mg, 0.01 eq), NiCl₂·dtbbpy (1.19 mg, 0.015 eq), TTMSS (49.73 mg, 1 eq), Na₂CO₃ (42.40 mg, 2 eq) in DME (2 mL, 0.1 M). The vial was sealed and placed under nitrogen was added. The reaction was stirred and irradiated with a 10 W blue LED lamp (3 cm away), with cooling water to keep the reaction temperature at 25° C. for 14 h. The mixture was diluted with water (50 mL) and extracted with EtOAc (50 mL×2). The combined organic layers were washed with saturated brine (40 mL×2), dried over Na₂SO₄, filtered and concentrated under reduced pressure to give a residue. The residue was purified by prep—TLC and concentrated under reduced pressure to give tert-butyl 3-(4-(dimethylamino)pyrimidin-5-yl)azetidine-1-carboxylate (40 mg, 0.14 mmol, 29% yield) as a yellow oil. LCMS (ESI) m/z=279.1 [M+H]+. 1H NMR (400 MHz, \hat{ }c) 8.57 (s, 1H), 8.31 (s, 1H), 4.35-4.26 (m, 2H), 3.97-3.93 (m, 2H), 3.37-3.34 (m, 1H), 3.25 (s, 6H), 1.42 (s, 9H).

### Step 3: Preparation of 5-(azetidin-3-yl)-N,N-dimethylpyrimidin-4-amine

To a solution of tert-butyl 3-(4-(dimethylamino)pyrimidin-5-yl)azetidine-1-carboxylate (40 mg, 0.14 mmol, 1 eq) in methylene chloride (0.60 mL) was added TFA (0.20 mL). The mixture was stirred at 25° C. for 1 h to give a yellow solution. The reaction mixture was concentrated under reduced pressure to give 5-(azetidin-3-yl)-N,N-dimethylpyrimidin-4-amine (26 mg, crude) as a yellow oil.

### Synthesis of 5-(azetidin-3-yl)-4-methoxypyrimidine

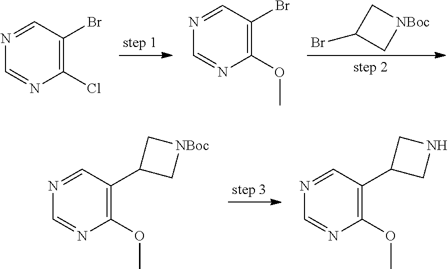

### Step 1: Preparation of 5-bromo-4-methoxypyrimidine

To a solution of 5-bromo-4-chloropyrimidine (1 g, 5.16 mmol, 1 eq) in MeOH (10 mL) was added tBuOK (694 mg, 6.19 mmol, 1.2 eq), the mixture was stirred at 25° C. for 2 h. The mixture was diluted with water (20 mL) and extracted with EtOAc (20 mL×3), the combined organic layers were washed with saturated brine (20 mL×2), dried over Na2SO4, filtered and concentrated under reduced pressure to give a white solids. The residue was purified by prep-TLC to give 5-bromo-4-methoxypyrimidine (864 mg, 4.57 mmol, 88.6% yield) as a white solid. LCMS (ESI) m/z=190.1 [M+H]+.

### Step 2: Preparation of tert-butyl 3-(4-methoxypyrimidin-5-yl)azetidine-1-carboxylate

To an 60 mL vial equipped with a stir bar was added 5-bromo-4-methoxypyrimidine (450 mg, 2.38 mmol, 1 eq), tert-butyl 3-bromoazetidine-1-carboxylate (561 mg, 2.38 mmol, eq), TTMSS (49.8 mg, 200 μmol, 1.0 eq), Na2CO3 (504 mg, 4.76 mmol, 2 eq), Ir[dF(CF₃)ppy]₂(dtbpy)(PF₆) (29.5 mg, 23.8 μmol, 0.01 eq), TTMSS (592 mg, 2.38 mmol, 1 eq) in DME (20 mL). The vial was sealed and placed under nitrogen was added. The reaction was stirred and irradiated with a 10 W blue LED lamp (3 cm away), with cooling water to keep the reaction temperature at 25° C. for 14 h. The reaction mixture was concentrated under reduced pressure to give a residue. The residue was purified by column chromatography to give tert-butyl 3-(4-methoxypyrimidin-5-yl)azetidine-1-carboxylate (350 mg, 1.31 mmol, 55.4% yield) as a yellow oil. LCMS (ESI) m/z=266.2 [M+H]⁺.

### Step 3: Preparation of 5-(azetidin-3-yl)-4-methoxypyrimidine

To a solution of tert-butyl 3-(4-methoxypyrimidin-5-yl)azetidine-1-carboxylate (350 mg, 1.31 mmol, 1 eq) in DCM (0.3 mL) and TFA (0.1 mL), the mixture was stirred at 25° C. for 1 h. The reaction mixture was concentrated under reduced pressure to give 5-(azetidin-3-yl)-4-methoxypyrimidine (216 mg, crude) as a yellow oil. LCMS (ESI) m/z=166.2 [M+H]+.

### Synthesis of 5-(azetidin-3-yl)pyrimidin-4-ol

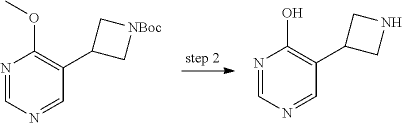 [SMILES: COc1ncncc1C1CN(C(=O)OC(C)(C)C)C1 ; Oc1ncncc1C1CNC1]

### Step 1: Preparation of 5-(azetidin-3-yl)pyrimidin-4-ol

To a solution of tert-butyl 3-(4-methoxypyrimidin-5-yl)azetidine-1-carboxylate (0.14 g, 0.53 mmol, 1 eq) in ACN (3 mL) was added 40% HBr/AcOH (1 mL, 0.53 mmol, 1.0 eq) to the mixture at 25° C., the mixture was stirred at 70° C. for 12 h to give a yellow suspension. The mixture was filtrated and the filtrate was concentrated under reduced pressure to give crude product as white solids. The solid was trituration by DCM (10 mL×3), then the mixture was filtered to give a white solid. The solid was concentrated under reduced pressure to give 5-(azetidin-3-yl)pyrimidin-4-ol (30 mg, crude) as a white solid. 1HNMR (400 MHz, DMSO-d6) δ 8.95-8.58 (m, 1H), 8.05-7.98 (m, 1H), 8.54-7.96 (m, 1H), 4.27-4.07 (m, 4H), 3.98-3.81 (m, 1H), 2.50 (s, 1H).

### Synthesis of 4-(4-fluoro-1H-pyrazol-3-yl)piperidine

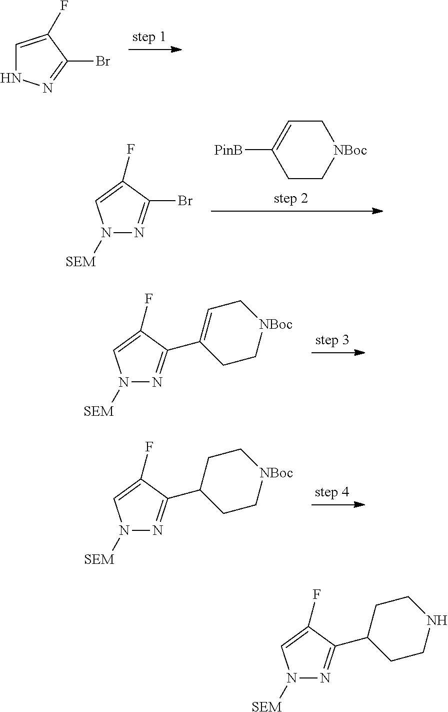

### Step 1: Preparation of 3-bromo-4-fluoro-1-((2-(trimethylsilyl)ethoxy)methyl)-1H-pyrazole

To a solution of 3-bromo-4-fluoro-1H-pyrazole (1.80 g, 11 mmol, 1 eq) in DMF (18 mL) was added NaH (0.34 g, 14 mmol, 1.3 eq) and SEMCl (2.4 g, 14 mmol, 1.3 eq) at 0° C. The mixture was stirred at 25° C. for 12 h. The reaction mixture was quenched by ice water. The mixture was diluted with water (0 mL) and extracted with EtOAc (50 mL×3). The combined organic layers were washed with brine (50 mL×3), dried over anhydrous Na2SO4, filtered and concentrated under reduced pressure to give a residue. The residue was purified by flash silica gel chromatography to give 3-bromo-4-fluoro-1-((2-(trimethylsilyl)ethoxy)methyl)-1H-pyrazole (406 mg, 1.4 mmol, 13% yield) as a colorless oil. 1H NMR (400 MHz, CDCl3) δ 7.45 (d, J=4.8 Hz, 1H), 5.41 (d, J=1.2 Hz, 2H), 3.57 (dd, J=7.6, 8.8 Hz, 2H), 0.92-0.88 (m, 2H), −0.03 (s, 9H).

### Step 2: Preparation of tert-butyl 4-(4-fluoro-1-((2-(trimethylsilyl)ethoxy)methyl)-1H-pyrazol-3-yl)-5,6-dihydropyridine-1(2H)-carboxylate

A mixture of 3-bromo-4-fluoro-1-{[2-(trimethylsilyl)ethoxy]methyl}-1H-pyrazole (350 mg, 1.18 mmol, 1 eq), tert-butyl 4-(4,4,5,5-tetramethyl-1,3,2-dioxaborolan-2-yl)-3,6-dihydropyridine-1(2H)-carboxylate (435 mg, 1.41 mmol, 1.2 eq), palladium(2+) bis((cyclopenta-1,3-dien-1-yl)diphenylphosphane) dichloromethane iron dichloride (193 mg, 236 μmol, 0.2 eq) and K2CO3 (489 mg, 3.54 mmol, 3.0 eq) in dioxane (3.5 mL) and water (0.9 mL) was degassed and purged with N2 (3 times). The mixture was stirred at 60° C. for 2 h under N2 atmosphere. The mixture was diluted with water (10 mL) and extracted with EtOAc (20 mL×3). The combined organic layers were washed with brine (20 mL×3), dried over anhydrous Na2SO4, filtered and concentrated under reduced pressure to give a residue. The residue was purified by flash silica gel chromatography to give tert-butyl 4-(4-fluoro-1-((2-(trimethylsilyl)ethoxy)methyl)-1H-pyrazol-3-yl)-5,6-dihydropyridine-1(2H)-carboxylate (305 mg, 767 μmol, 65% yield) as a yellow oil. 1H NMR (400 MHz, CDCl3) δ 7.35 (d, J=4.4 Hz, 1H), 6.18 (s, 1H), 5.34 (s, 2H), 4.12-4.10 (m, 2H), 3.66-3.61 (m, 4H), 2.50 (s, 2H), 1.49 (s, 9H), 0.91-0.87 (m, 2H), −0.02 (s, 9H).

### Step 3: Preparation of tert-butyl 4-(4-fluoro-1-((2-(trimethylsilyl)ethoxy)methyl)-1H-pyrazol-3-yl)piperidine-1-carboxylate

To a solution of tert-butyl 4-(4-fluoro-1-((2-(trimethylsilyl)ethoxy)methyl)-1H-pyrazol-3-yl)-5,6-dihydropyridine-1(2H)-carboxylate (300 mg, 754 μmol, 1.0 eq) in methanol (3 mL) was added wet Pd/C (10%, 30 mg) under N₂ atmosphere. The suspension was degassed and purged with H2 (3 times). The mixture was stirred under H2 (15 Psi) at 25° C. for 2 h. The reaction solution was filtered and filtrate was concentrated under pressure to give tert-butyl 4-(4-fluoro-1-((2-(trimethylsilyl)ethoxy)methyl)-1H-pyrazol-3-yl)piperidine-1-carboxylate (230 mg, crude) as a white solid. ¹H NMR (400 MHz, CD₃OD) δ 7.36 (d, J=4.0 Hz, 1H), 5.42 (s, 2H), 4.85 (s, 2H), 4.19 (d, J=13.2 Hz, 2H), 3.55-3.50 (m, 1H), 3.34-3.28 (m, 2H), 3.13-3.05 (m, 1H), 2.91-2.79 (m, 2H), 1.77 (d, J=4.0 Hz, 1H), 1.48 (s, 9H), 0.89-0.85 (m, 2H), −0.02 (s, 9H).

### Step 4: Preparation of 4-(4-fluoro-1H-pyrazol-3-yl)piperidine

To a solution of tert-butyl 4-(4-fluoro-1-((2-(trimethylsilyl)ethoxy)methyl)-1H-pyrazol-3-yl)piperidine-1-carboxylate (100 mg, 250 μmol, 1.0 eq) in DCM (1 mL) was added TFA (0.3 mL). The mixture was stirred at 25° C. for 0.5 h. The reaction mixture was concentrated under reduced pressure to give 4-(4-fluoro-1H-pyrazol-3-yl)piperidine (60 mg, crude) as a yellow oil.

### Synthesis of 4-(1H-pyrazol-3-yl)piperidine

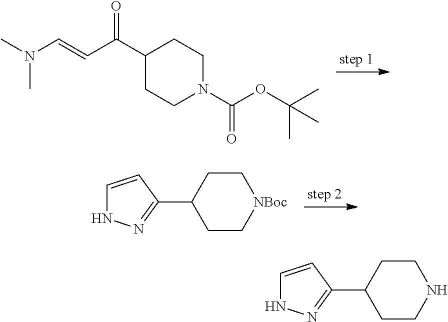

### Step 1: Preparation of Tert-Butyl 4-(1H-pyrazol-3-yl)piperidine-1-carboxylate

To a solution of tert-butyl 4-[(2E)-3-(dimethylamino)prop-2-enoyl]piperidine-1-carboxylate (0.5 g, 1.8 mmol, 1 eq) in EtOH (10 mL) was added hydrazine hydrate (0.13 g, 2.7 mmol, 1.5 eq) at 0° C., the mixture was stirred at 80° C. for 16 h. The reaction mixture was concentrated under reduced pressure to give a residue. The residue was diluted with water (50 mL) and extracted with EtOAc (50 mL×2), the combined organic layers were washed with saturated brine (50 mL×2), dried over Na2SO4, filtered and concentrated under reduced pressure to give a residue. The residue was purified by flash silica gel chromatography to give tert-butyl 4-(1H-pyrazol-3-yl)piperidine-1-carboxylate (0.4 g, 1.6 mmol, 90% yield) as a yellow oil. LCMS (ESI) m/z=196.3 [M+H]⁺. ¹H NMR (400 MHz, CDCl₃) δ 7.52 (d, J=2.0 Hz, 1H), 6.13 (d, J=2.0 Hz, 1H), 2.86 (ddd, J=3.7, 8.0, 15.4 Hz, 4H), 2.05 (s, 1H), 2.00-1.92 (m, 2H), 1.70-1.58 (m, 2H), 1.48 (s, 9H)

### Step 2: Preparation of 4-(1H-pyrazol-3-yl)piperidine

To a solution of tert-butyl 4-(1H-pyrazol-3-yl)piperidine-1-carboxylate (50 mg, 0.20 mmol, 1 eq) in DCM (0.6 mL) was added TFA (0.2 mL), the mixture was stirred at 25° C. for 10 min to give a colorless clean solution. The reaction mixture was concentrated under reduced pressure to give 4-(1H-pyrazol-3-yl)piperidine (30 mg, crude) as a white oil. LCMS (ESI) m/z=152.0 [M+H]+.

### Synthesis of 4-(azetidin-3-yl)-1-methyl-1H-pyrazol-5-ol

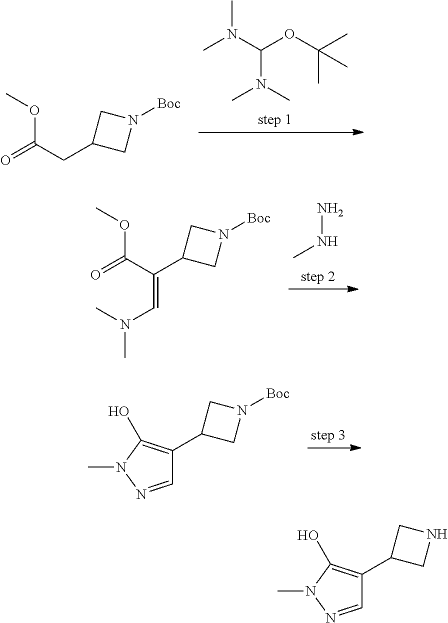

### Step 1: Preparation of Tert-Butyl (Z)-3-(1-(dimethylamino)-3-methoxy-3-oxoprop-1-en-2-yl)azetidine-1-carboxylate

To a solution of tert-butyl 3-(2-methoxy-2-oxoethyl)azetidine-1-carboxylate (2.5 g, 11 mmol, 1 eq), 1-tert-butoxy-N,N,N′,N′-tetramethylmethanediamine (1.9 g, 11 mmol, 1 eq) in DMF (20 ml). The mixture was stirred at 100° C. for 12 h. The mixture was diluted with water (100 mL) and extracted with EtOAc (100 mL×3). The combined organic layers were washed with brine (100 mL×3), dried over anhydrous Na2SO4, filtered and concentrated under reduced pressure to give a residue. The residue was purified by column chromatography to give tert-butyl 3-[(1Z)-1-(dimethylamino)-3-methoxy-3-oxoprop-1-en-2-yl]azetidine-1-carboxylate (1.30 g, 4.6 mmol, 42% yield) as a yellow solid. 1H NMR (400 MHz, CDCl3) δ 7.64 (s, 1H), 3.92 (s, 4H), 3.62-3.57 (m, 3H), 3.33 (s, 7H), 31-1.17 (m, 9H)

### Step 2: Preparation of tert-butyl 3-(5-hydroxy-1-methyl-1H-pyrazol-4-yl)azetidine-1-carboxylate

To a solution of tert-butyl 3-[(1Z)-1-(dimethylamino)-3-methoxy-3-oxoprop-1-en-2-yl]azetidine-1-carboxylate (1.5 g, 5.28 mmol, 1 eq), methylhydrazine (530 mg, 11.6 mmol, 2.2 eq) in EtOH (12 mL). The mixture was stirred at 80° C. for 3 h. The reaction mixture was concentrated under reduced pressure to give a residue. The residue was purified by reversed phase (TFA) then lyophilized to give tert-butyl 3-(5-hydroxy-1-methyl-1H-pyrazol-4-yl)azetidine-1-carboxylate (0.75 g, 3.0 mmol, 56% yield) as a yellow solid. LCMS (ESI) m/z=254.2 [M+H]+.

### Step 3: Preparation of 4-(azetidin-3-yl)-1-methyl-1H-pyrazol-5-ol

To a solution of tert-butyl 3-(5-hydroxy-1-methyl-1H-pyrazol-4-yl)azetidine-1-carboxylate (750 mg, 2.96 mmol, 1 eq) in methylene chloride (1 mL) was added TFA (0.3 mL). The mixture was stirred at 25° C. for 1 h. The reaction mixture was concentrated under reduced pressure to give 4-(azetidin-3-yl)-1-methyl-1H-pyrazol-5-ol (450 mg, crude) as a yellow oil. LCMS (ESI) m/z=154.2 [M+H]⁺.

### Synthesis of 4-(azetidin-3-yl)-5-methoxy-1-methyl-1H-pyrazole

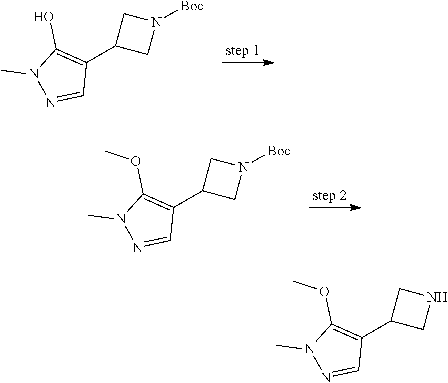 [SMILES: Cn1ncc(C2CN(C(=O)OC(C)(C)C)C2)c1O ; COc1c(C2CN(C(=O)OC(C)(C)C)C2)cnn1C ; COc1c(C2CNC2)cnn1C]

### Step 1: Preparation of Tert-Butyl 3-(5-methoxy-1-methyl-1H-pyrazol-4-yl)azetidine-1-carboxylate

To a solution of tert-butyl 3-(5-hydroxy-1-methyl-1H-pyrazol-4-yl)azetidine-1-carboxylate (1.1 g, 4.2 mmol, 1 eq) in ACN (10 mL) was added K2CO₃ (1.2 g, 8. mmol, 2.0 eq) and iodomethane (0.60 g, 4.2 mmol, 1 eq). The mixture was stirred at 25° C. for 3 h. The reaction mixture was filter and the filtrate was concentrated to give a residue. The residue was purified by reversed phase (TFA) then lyophilized to give tert-butyl 3-(5-methoxy-1-methyl-1H-pyrazol-4-yl)azetidine-1-carboxylate (60 mg, 0.22 mmol, 5.4% yield) as a yellow oil. 1H NMR (400 MHz, CDCl3) δ 7.38 (s, 1H), 4.25 (t, J=8.6 Hz, 2H), 3.94 (dd, J=6.5, 8.4 Hz, 3H), 3.86 (s, 3H), 3.68 (s, 3H), 1.46 (s, 9H).

### Step 2: Preparation of 4-(azetidin-3-yl)-5-methoxy-1-methyl-1H-pyrazole

To a solution of tert-butyl 3-(5-methoxy-1-methyl-1H-pyrazol-4-yl) azetidine-1-carboxylate (60 mg, 224 μmol, 1 eq) in methylene chloride (1 mL) was added TFA (0.3 mL). The mixture was stirred at 25° C. for 1 h and subsequently concentrated under reduced pressure to give 4-(azetidin-3-yl)-5-methoxy-1-methyl-1H-pyrazole (30 mg, crude) as a yellow oil.

### Synthesis of (3S,4S)-4-fluoro-1-(1-((2-(trimethylsilyl)ethoxy)methyl)-1H-imidazol-2-yl)pyrrolidin-3-amine

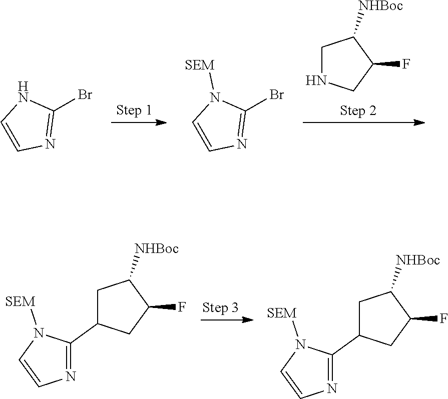 [SMILES: Brc1ncc[nH]1 ; C[Si](C)(C)CCOCn1ccnc1Br ; CC(C)(C)OC(=O)N[C@H]1CNC[C@@H]1F ; CC(C)(C)OC(=O)N[C@H]1CC(c2nccn2COCC[Si](C)(C)C)C[C@@H]1F ; CC(C)(C)OC(=O)N[C@H]1CC(c2nccn2COCC[Si](C)(C)C)C[C@@H]1F]

### Step 1: Preparation of 2-bromo-1-((2-(trimethylsilyl)ethoxy)methyl)-1H-imidazole

A solution of 2-bromo-1H-imidazole (4 g, 27.2 mmol, 1 eq) in THF (50 mL) was added NaH (1.3 g, 33 mmol, 1.20 eq) at 0° C. To the mixture was added SEMCl (5.9 g, 35 mmol, 1.3 eq) and stirred at 25° C. for 1 h. The reaction mixture was quenched with H2O (20 mL) at 0° C. and extracted with EtOAc (20 mL×3). The combined organic layers were washed with brine (20 mL×3), dried over anhydrous Na2SO4, filtered, and concentrated under reduced pressure to give a residue. The residue was purified by flash silica gel chromatography to give 2-bromo-1-((2-(trimethylsilyl)ethoxy)methyl)-1H-imidazole (1.3 g, 4.7 mmol, 17% yield) as a yellow oil. 1H NMR (400 MHz, CDCl3) δ 7.27-7.21 (m, 2H), 5.43 (s, 2H), 3.72-3.67 (m, 2H), 1.09-1.06 (m, 2H), 0.15 (s, 9H).

### Step 2: Preparation of Tert-Butyl ((3S,4S)-4-fluoro-1-(1-((2-(trimethylsilyl)ethoxy)methyl)-1H-imidazol-2-yl)pyrrolidin-3-yl)carbamate

A solution of 2-bromo-1-((2-(trimethylsilyl)ethoxy)methyl)-1H-imidazole (800 mg, 2.88 mmol, 1 eq), tert-butyl N-[(3S,4S)-4-fluoropyrrolidin-3-yl]carbamate (1.17 g, 5.76 mmol, 2 eq), tBuONa (690 mg, 7.19 mmol, 2.5 eq) in toluene (10 mL) was added {1,3-bis[2,6-bis(pentan-3-yl)phenyl]-4,5-dichloro-2,3-dihydro-1H-imidazol-2-ylidene}dichloro(3-chloropyridin-1-ium-1-yl)palladium (123 mg, 144 μmol, 0.05 eq). The mixture was stirred at 85° C. for 2 h. The reaction mixture was concentrated under reduced pressure to give a residue. The residue was purified by flash silica gel chromatography to give tert-butyl N-[(3S,4S)-4-fluoro-1-(1-{[2-(trimethylsilyl)ethoxy]methyl}-1H-imidazol-2-yl)pyrrolidin-3-yl]carbamate (918 mg, 2.29 mmol, 79.8% yield) as a yellow oil. LCMS (ESI) m/z=401.1 [M+H]+.

### Step 3: Preparation of (3S,4S)-4-fluoro-1-(1-((2-(trimethylsilyl)ethoxy)methyl)-1H-imidazol-2-yl)pyrrolidin-3-amine

A solution of tert-butyl N-[(3S,4S)-4-fluoro-1-(1-{[2-(trimethylsilyl)ethoxy]methyl}-1H-imidazol-2-yl)pyrrolidin-3-yl]carbamate (900 mg, 2.24 mmol, 1 eq) in DCM (9 mL) was added TFA (3 mL). The mixture was stirred at 25° C. for 0.5 h. The reaction mixture was concentrated under reduced pressure to give (3S,4S)-4-fluoro-1-(1-((2-(trimethylsilyl)ethoxy)methyl)-1H-imidazol-2-yl)pyrrolidin-3-amine (918 mg, crude) as a yellow oil. LCMS (ESI) m/z=301.1 [M+H]⁺.

### Synthesis of 5-(azetidin-3-yl)-4-methylpyridin-2-ol

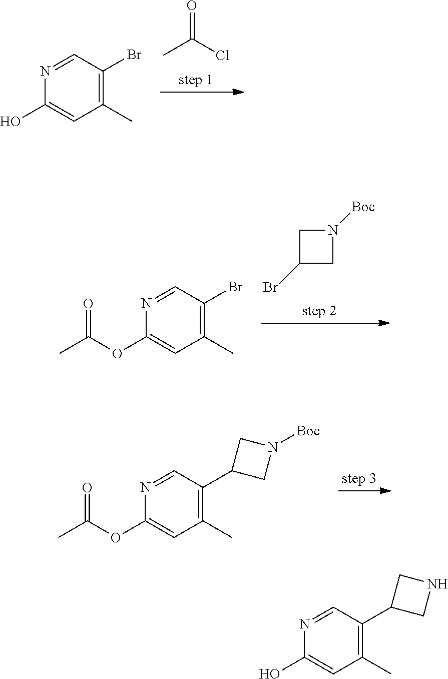

### Step 1: Preparation of 5-bromo-4-methylpyridin-2-yl acetate

To a solution of 5-bromo-4-methylpyridin-2-ol (1.0 g, 5.3 mmol, 1.0 eq) in DCM (10 mL) was added triethylamine (1.3 g, 13 mmol, 2.5 eq), then a solution of acetyl chloride (2.1 g, 26 mmol, 5.0 eq) in DCM (10 mL) was added at 0° C., the mixture was stirred at 25° C. for 12 h to give a brown suspension. The reaction mixture was concentrated under reduced pressure to give a residue. The residue was purified by flash silica gel chromatography to give 5-bromo-4-methylpyridin-2-yl acetate (0.69 g, 3 mmol, 57% yield) as a white oil. 1H NMR (400 MHz, CDCl3) δ 8.42 (s, 1H) 6.98 (s, 1H) 2.42 (s, 3H) 2.33 (s, 3H).

### Step 2: Preparation of tert-butyl 3-(6-acetoxy-4-methylpyridin-3-yl)azetidine-1-carboxylate

To an 15 mL vial equipped with a stir bar was added 5-bromo-4-methylpyridin-2-yl acetate (0.50 g, 2.2 mmol, 1.0 eq), tert-butyl 3-bromoazetidine-1-carboxylate (0.67 g, 2.8 mmol, 1.3 eq), Ir[dF(CF3)ppy]2(dtbpy)(PF6) (0.27 g, 0.22 mmol, 0.10 eq), NiCl2·dtbbpy (13 mg, 33 μmol, 0.015 eq), TTMSS (0.54 g, 2.2 mmol, 1.0 eq), Na2CO3 (0.46 g, 4.3 mmol, 2.0 eq) in DME (22 mL). The vial was sealed and placed under nitrogen. The reaction was stirred and irradiated with a 10 W blue LED lamp (3 cm away), with cooling water to keep the reaction temperature at 25° C. for 14 h to give a brown suspension solution. The reaction mixture was filtered and concentrated under reduced pressure to give a residue. The residue was purified by flash silica gel chromatography to give tert-butyl 3-(6-acetoxy-4-methylpyridin-3-yl)azetidine-1-carboxylate (0.40 g, 1.3 mmol, 60% yield) as a yellow oil. 1H NMR (400 MHz, CDCl3) δ 7.22-7.26 (m, 1H) 6.40 (s, 1H) 4.23 (t, J=8.00 Hz, 2H) 3.84-3.88 (m, 2H) 3.68 (m, 1H) 2.18-2.32 (m, 3H) 2.10 (s, 3H) 1.44 (s, 9H).

### Step 3: Preparation of 5-(azetidin-3-yl)-4-methylpyridin-2-ol

To a solution of tert-butyl 3-[6-(acetyloxy)-4-methylpyridin-3-yl]azetidine-1-carboxylate (0.20 g, 0.65 mmol, 1.0 eq) in ACN (5.0 mL) was added 1 M HCl (5.0 mL). Then the mixture was stirred at 80° C. for 12 h to give a yellow solution. The reaction mixture was concentrated under reduced pressure to give 5-(azetidin-3-yl)-4-methylpyridin-2-ol (0.20 g, crude) as a yellow oil. LCMS (ESI) m/z=165.1 [M+H]+.

### Synthesis of tert-butyl 3-(4-fluoro-2-methylpyridin-3-yl)azetidine-1-carboxylate

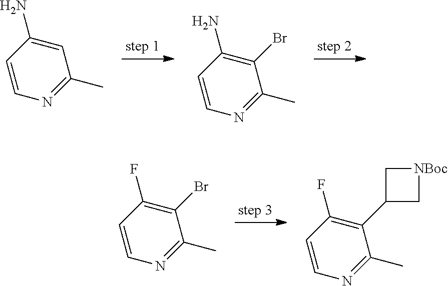

### Step 1: Preparation of 3-bromo-2-methylpyridin-4-amine

To a solution of 2-methylpyridin-4-amine (10 g, 92 mmol, 1 eq) in hydrogen bromide (100 mL, 40% in water) was dropwise added peroxol (21 g, 0.18 mol, 2 eq, 30% wt) at 40° C., then the mixture solution was stirred at 60° C. for 1 h to give a red suspension. To the mixture was added sufficient saturated NaHCO₃(300 mL) to adjust pH˜8. The resulting mixture was extracted with EtOAc (400 mL×2), the combined organic layers were dried over Na₂SO₄, filtered and concentrated under reduced pressure to give a residue. The residue was purified by column chromatography to give a 3-bromo-2-methylpyridin-4-amine (10 g, 53 mmol, 58% yield) as a white solid. 1H NMR (400 MHz, CDCl3) δ 7.98 (d, J=5.6 Hz, 1H), 6.45 (d, J=5.6 Hz, 1H), 4.71 (s, 2H), 2.59 (s, 3H)

### Step 2: Preparation of 3-bromo-4-fluoro-2-methylpyridine

To cooled (0° C.) pyridine·HF (2 mL) was added 3-bromo-2-methylpyridin-4-amine (0.22 g, 1.2 mmol, 1 eq) slowly, followed by slow portion wise addition of sodium nitrite (0.11 g, 1.6 mmol, 1.4 eq). The mixture solution was stirred at 60° C. for 2 h to give a yellow suspension. The reaction mixture was diluted with NaHCO₃(20 mL) and extracted with EtOAc (20 mL×2). The combined organic layers were dried over Na₂SO₄, filtered, and concentrated under reduced pressure to give a residue. The residue was purified by column chromatography to give 3-bromo-4-fluoro-2-methylpyridine (0.11 g, 0.63 mmol, 54% yield) as a yellow oil. LCMS (ESI) m/z=191.9 [M+H]+.

### Step 3: Preparation of tert-butyl 3-(4-fluoro-2-methylpyridin-3-yl)azetidine-1-carboxylate

To a solution of 5-bromo-4-fluoro-2-methylpyridine (0.10 g, 0.53 mmol, 1 eq), tert-butyl 3-bromoazetidine-1-carboxylate (0.16 g, 0.68 mmol, 1.3 eq) in DME (10 mL) was added Ir[dF(CF3)ppy]2(dtbpy)(PF₆) (27 mg, 24 μmol, 0.05 eq), iridium(1+) bis(2-phenylpyridine) 4,4 (33 mg, 36 μmol, 0.068 eq), K2CO3 (86 mg, 1.06 mmol, 2 eq) and TTMSS (0.60 g, 0.53 mmol, 1.0 eq). The resulting solution was stirred and irradiated with a 10 W blue LED lamp ((3 cm away), with cooling water to keep the reaction temperature at 25° C. After stirring for 14 h, the reaction mixture produced brown suspension. The reaction mixture was diluted with water (10 mL) and extracted with EtOAc (10 mL×2), the combined organic layers were washed with saturated brine (10 mL×2), dried over Na₂SO₄, filtered, and concentrated under reduced pressure to give a residue. The residue was purified by column chromatography to give tert-butyl 3-(4-fluoro-2-methylpyridin-3-yl)azetidine-1-carboxylate (50 mg, 0.19 mmol, 36% yield) as a yellow oil. LCMS (ESI) m/z=267.2 [M+H]+.

### Synthesis of tert-butyl 3-(4-cyclopropylpyridin-3-yl)azetidine-1-carboxylate

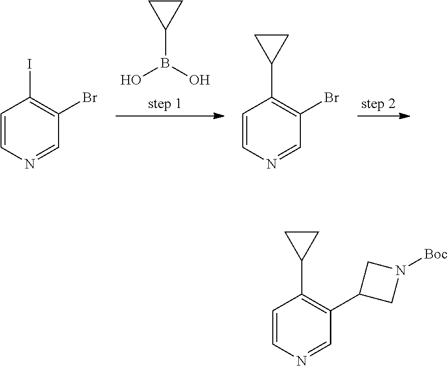 [SMILES: Brc1cnccc1I ; OB(O)C1CC1 ; Brc1cnccc1C1CC1 ; CC(C)(C)OC(=O)N1CC(c2cnccc2C2CC2)C1]

### Step 1: Preparation of 3-bromo-4-cyclopropylpyridine

To a solution of 3-bromo-4-iodopyridine (2.0 g, 7.0 mmol, 1 eq) and cyclopropylboronic acid (0.6 g, 7.0 mmol, 1 eq) in mixture of 1,4-dioxane (40 mL) and water (10 mL) was added Na2CO3 (2.22 g, 21 mmol, 3 eq) and palladium(4+) bis((cyclopenta-2,4-dien-1-ylidene)diphenylphosphanuide) iron dichloride (0.5 g, 0.7 mmol, 0.1 eq) under N₂ atmosphere. The mixture solution was stirred at 100° C. for 12 h to give a green suspension. The reaction mixture was concentrated under reduced pressure to give a residue. The residue was diluted with water (30 mL) and extracted with EtOAc (50 mL×2), the combined organic layers were washed with saturated brine (60 mL×2), dried over Na2SO4, filtered, and concentrated under reduced pressure to give a residue. The residue was purified by column chromatography to give 3-bromo-4-cyclopropylpyridine (480 mg, 2.4 mmol, 34% yield) as a yellow oil. 1H NMR (400 MHz, CDCl3) δ 8.63 (s, 1H), 8.40-8.28 (m, 1H), 6.73 (d, J=5.2 Hz, 1H), 2.23 (dd, J=5.2, 8.4 Hz, 1H), 1.19-1.12 (m, 2H), 0.82-0.76 (m, 2H).

### Step 2: Preparation of tert-butyl 3-(4-cyclopropylpyridin-3-yl)azetidine-1-carboxylate

To a solution of 3-bromo-4-cyclopropylpyridine (0.48 g, 2.4 mmol, 1 eq), tert-butyl 3-bromoazetidine-1-carboxylate (0.73 g, 3.1 mmol, 1.3 eq) in DME (20 mL) was added Ir[dF(CF3)ppy]2(dtbpy)(PF6) (27 mg, 24 μmol, 0.01 eq), iridium(1+) bis(2-phenylpyridine) 4,4 (33 mg, 36 μmol, 0.015 eq), Na2CO3 (0.51 g, 4.8 mmol, 2 eq) and TTMSS (0.60 g, 2.4 mmol, 1.0 eq). The resulting solution was stirred and irradiated with a 10 W blue LED lamp (3 cm away), with cooling water to keep the reaction temperature at 25° C. After stirring for 14 h, the formation of brown suspension was observed. The reaction mixture was concentrated under reduced pressure to give a residue. The residue was diluted with water (60 mL) and extracted with EtOAc (60 mL×2). The combined organic layers were washed with saturated brine (80 mL×2), dried over Na2SO4, filtered, and concentrated under reduced pressure to give a residue. The residue was purified by column chromatography to give tert-butyl 3-(4-cyclopropylpyridin-3-yl)azetidine-1-carboxylate (0.32 g, 1.2 mmol, 49% yield) as a yellow oil. LCMS (ESI) m/z=275.1 [M+H]+.

### Synthesis of 3-(azetidin-3-yl)pyrimidin-4(3H)-one

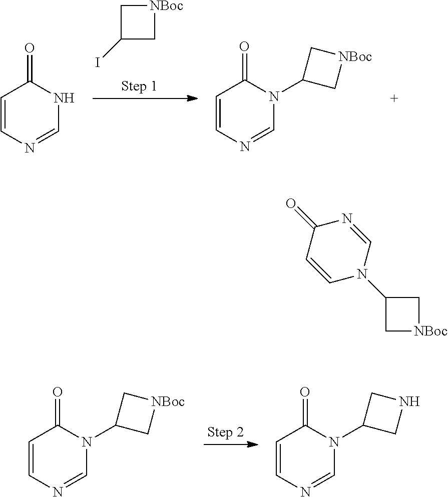 [SMILES: O=c1ccnc[nH]1 ; CC(C)(C)OC(=O)N1CC(I)C1 ; CC(C)(C)OC(=O)N1CC(n2cnccc2=O)C1 ; CC(C)(C)OC(=O)N1CC(n2ccc(=O)nc2)C1 ; CC(C)(C)OC(=O)N1CC(n2cnccc2=O)C1 ; O=c1ccncn1C1CNC1]

### Step 1: Preparation of Tert-Butyl 3-(6-oxopyrimidin-1(6H)-yl)azetidine-1-carboxylate

To a solution of pyrimidin-4(3H)-one (0.5 g, 5.2 mmol, 1 eq) in DMF (10 mL) was added tert-butyl 3-iodoazetidine-1-carboxylate (1.5 g, 5.2 mmol, 1 eq) and K2CO3 (1.1 g, 7.8 mmol, 1.5 eq). The mixture was stirred at 80° C. for 12 h to give a yellow solution under N2. The mixture was diluted with water (30 mL) and extracted with EtOAc (30 mL×2). The combined organic layers were washed with saturated brine (30 mL×2), dried over Na2SO4, filtered and concentrated under reduced pressure to give a residue. The residue was purified by flash silica gel chromatography to give tert-butyl 3-(6-oxopyrimidin-1(6H)-yl)azetidine-1-carboxylate (0.25 g, 0.85 mmol, 16% yield) as yellow solid and tert-butyl 3-(4-oxopyrimidin-1(4H)-yl)azetidine-1-carboxylate (0.7 g, 2.6 mmol, 49% yield) as yellow solid.

tert-butyl 3-(6-oxopyrimidin-1(6H)-yl)azetidine-1-carboxylate: LCMS (ESI) m/z=252.1 [M+H]+. 1H NMR (400 MHz, CD3OD) δ 8.41 (s, 1H), 7.94 (d, J=6.4 Hz, 1H), 6.45-6.48 (m, 1H), 5.09-5.12 (m, 1H), 4.49-4.21 (m, 4H), 1.47 (s, 9H).

tert-butyl 3-(4-oxopyrimidin-1(4H)-yl)azetidine-1-carboxylate: LCMS (ESI) m/z=252.1 [M+H]+. 1H NMR (400 MHz, CDCl3) δ 8.77 (s, 1H), 8.50 (d, J=5.6 Hz, 1H), 6.83 (d, J=5.6 Hz, 1H), 5.42-5.39 (m, 1H), 4.38-4.33 (m, 2H), 4.06-3.95 (m, 2H), 1.46 (s, 9H).

The following intermediates in Table 83 were prepared using the method described above in step 1 for the preparation of tert-butyl 3-(6-oxopyrimidin-1(6H)-yl)azetidine-1-carboxylate and utilizing the appropriate starting materials and modifications.

TABLE 83

| Name | Structure | LCMS |
|---|---|---|
| 1-(azetidin-3-yl)- 1H-pyrazole | 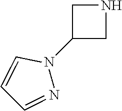 | 124.2 [M + H]⁺ |
| 1-(azetidin-3-yl)-4- fluoro-1H-pyrazole | 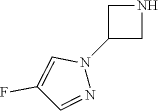 | 142.3 [M + H]⁺ |

### Step 2: Preparation of 3-(azetidin-3-yl)pyrimidin-4(3H)-one

To a solution of tert-butyl 3-(6-oxopyrimidin-1(6H)-yl)azetidine-1-carboxylate (100 mg, 0.40 mmol, 1 eq) in DCM (1.5 mL) was added TFA (0.5 mL). The mixture was stirred at 25° C. for 1 h to give a yellow solution. The reaction mixture was concentrated under reduced pressure to give 3-(azetidin-3-yl)pyrimidin-4(3H)-one (100 mg, crude) as a yellow oil. LCMS (ESI) m/z=152.1 [M+H]⁺.

### Synthesis of 3-(azetidin-3-yl)-5-fluoro-4-methoxypyridine

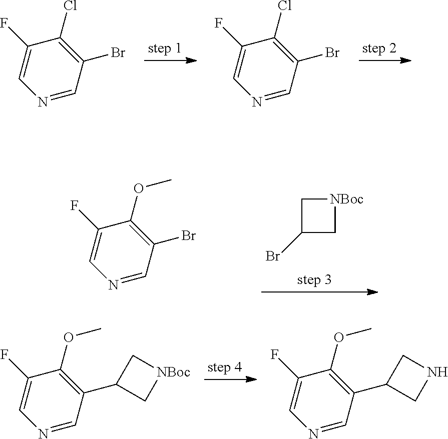

Step 1: Preparation of 3-bromo-4-chloro-5-fluoropyridine

To a cooled (−78° C.) solution of 3-bromo-5-fluoropyridine (10 g, 57 mmol, 1 eq) in THF (100 mL) was slowly added LiHMDS (25 mL, 62 mmol, 1.1 eq) over 0.5 h. The mixture was stirred at −78° C. for another 0.5 h, followed by slow addition of a solution of hexachloroethane (15 g, 62 mmol, 1.1 eq) in THF (100 mL). The mixture was stirred at −78° C. for 1 h and allowed to warm to 25° C. The mixture was stirred at 25° C. for 0.5 h and quenched by sat NH4Cl (50 mL) and extracted with EtOAc 90 mL (30 mL×3). The combined organic layers were washed with saturated brine (100 mL×2), dried over Na₂SO₄, filtered and concentrated under reduced pressure to give a residue. The residue was purified by flash silica gel chromatography to give 3-bromo-4-chloro-5-fluoropyridine (5.4 g, 26 mmol, 45% yield) as colorless oil was obtained. LCMS (ESI) m/z=210.1 [M+H]+.

### Step 2: Preparation of 3-bromo-5-fluoro-4-methoxypyridine

To a solution of 3-bromo-4-chloro-5-fluoropyridine (2.0 g, 9.5 mmol, 1.0 eq) in THF (10 mL) and MeOH (10 mL) was added NaOMe (0.5 g, 9.5 mmol, 1.0 eq). The mixture was stirred at 25° C. for 12 h. The reaction was filter and the filtrate was concentrated under reduced pressure to give a residue. The residue was purified by column chromatography to give 3-bromo-5-fluoro-4-methoxypyridine (900 mg, 4.4 mmol, 46% yield) was obtained as a yellow solid. LCMS (ESI) m/z=207.8 [M+H]+.

### Step 3: Preparation of tert-butyl 3-(5-fluoro-4-methoxypyridin-3-yl)azetidine-1-carboxylate

To a solution of 3-bromo-5-fluoro-4-methoxypyridine (0.7 g, 3.4 mmol, 1.0 eq) in DME (10 mL) was added tert-butyl 3-bromoazetidine-1-carboxylate (1.0 g, 4.4 mmol, 1.3 eq), Ir[dF(CF3)ppy]2(dtbpy)(PF6) (38 mg, 0.034 mmol, 0.01 eq), NiCl2·dtbbpy (11.1 mg, 0.051 mmol, 0.015 eq), TTMSS (842 mg, 3.4 mmol, 1.00 eq), Na2CO3 (718 mg, 6.8 mmol, 2 eq). The vial was sealed and placed under nitrogen was added. The reaction was stirred and irradiated with a 10 W blue LED lamp (3 cm away), with cooling water to keep the reaction temperature at 25° C. for 14 h. The reaction was diluted with water (30 mL) and extracted with EtOAc (30 mL×2). The combined organic layers were washed with saturated brine (50 mL×2), dried over Na2SO4, filtered and concentrated under reduced pressure to give a residue. The residue was purified by column chromatography to give tert-butyl 3-(5-fluoro-4-methoxypyridin-3-yl)azetidine-1-carboxylate (0.6 g, 2.1 mmol, 63% yield) as a white solid. LCMS (ESI) m/z=183.0 [M+H]+.

### Step 4: Preparation of 3-(azetidin-3-yl)-5-fluoro-4-methoxypyridine

To a solution of tert-butyl 3-(5-fluoro-4-methoxypyridin-3-yl)azetidine-1-carboxylate (50 mg, 177 μmol, 1 eq) in DCM (0.3 mL) and TFA (0.1 mL). The mixture was stirred at 25° C. for 1 h. The reaction mixture was concentrated under reduced pressure to give a residue 3-(azetidin-3-yl)-5-fluoro-4-methoxypyridine (32 mg, crude) as a yellow oil. LCMS (ESI) m/z=183.1 [M+H]+.

### Synthesis of 5-(azetidin-3-yl)-3-methylpyrimidin-4(3H)-one

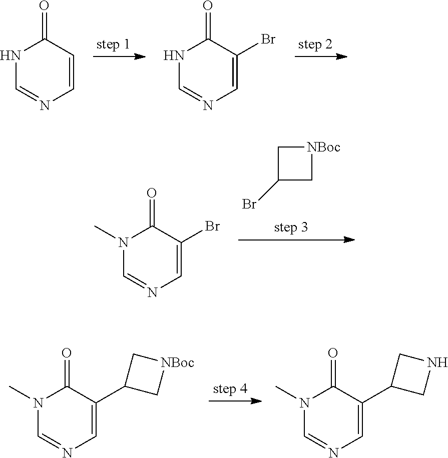

### Step 1: Preparation of 5-bromopyrimidin-4(3H)-one

To a solution of pyrimidin-4(3H)-one (3 g, 31 mmol, 1.0 eq) in chloroform (15 mL) was added bromine (1.6 mL, 31 mmol, 1 eq). The mixture was stirred at 20° C. for 12 h. The reaction was diluted with water (60 mL) and extracted with EtOAc (60 mL×2). The combined organic layers were washed with saturated brine (60 mL×2), dried over Na2SO4, filtered and concentrated under reduced pressure to give 5-bromopyrimidin-4(3H)-one (4.2 g, crude) as a yellow solid.

### Step 2: Preparation of 5-bromo-3-methylpyrimidin-4(3H)-one

To a solution of 5-bromopyrimidin-4(3H)-one5-bromopyrimidin-4(3H)-one (0.5 g, 2.9 mmol, 1.0 eq) in acetone (10 mL) was added K₂CO₃ (0.59 g, 4.3 mmol, 1.5 eq) and iodomethane (0.45 g, 3.1 mmol, 1.1 eq). The mixture was stirred at 25° C. for 12 h. The reaction was diluted with water (50 mL) and extracted with EtOAc (50 mL×2), the combined organic layers were washed with saturated brine (50 mL×2), dried over Na2SO4, filtered, and concentrated under reduced pressure to give a residue. The residue was purified by column chromatography to give 5-bromo-3-methylpyrimidin-4(3H)-one (100 mg, 529 μmol, 19% yield) as a white solid. LCMS (ESI) m/z=189.1 [M+H]+.

### Step 3: Preparation of tert-butyl 3-(1-methyl-6-oxo-1,6-dihydropyrimidin-5-yl)azetidine-1-carboxylate

To a solution of 5-bromo-3-methylpyrimidin-4(3H)-one (100 mg, 264 μmol, 1 eq) in DME (2 mL) was added tert-butyl 3-bromoazetidine-1-carboxylate (162 mg, 343 μmol, 1.3 eq), Ir[dF(CF₃)ppy]₂(dtbpy)(PF₆) (5.9 mg, 2.6 μmol, 0.01 eq), NiCl₂·dtbbpy (1.7 mg, 3.7 μmol, 0.015 eq), TTMSS (131 mg, 264 μmol, 1.00 eq), Na₂CO₃ (112 mg, 264 μmol, 2 eq). The vial was sealed and placed under nitrogen was added. The reaction was stirred and irradiated with a 10 W blue LED lamp (3 cm away), with cooling water to keep the reaction temperature at 25° C. for 14 h. The reaction was diluted with water (10 mL) and extracted with EtOAc (10 mL×2), the combined organic layers were washed with saturated brine (10 mL×2), dried over Na₂SO₄, filtered and concentrated under reduced pressure to give a residue. The residue was purified by column chromatography to give tert-butyl 3-(1-methyl-6-oxo-1,6-dihydropyrimidin-5-yl)azetidine-1-carboxylate (106 mg, 199 μmol, 76% yield) as a white solid. LCMS (ESI) m/z=266.3 [M+H]⁺.

### Step 4: Preparation of 5-(azetidin-3-yl)-3-methylpyrimidin-4(3H)-one

To a solution of tert-butyl 3-(1-methyl-6-oxo-1,6-dihydropyrimidin-5-yl)azetidine-1-carboxylate (106 mg, 199 μmol, 1 eq) in DCM (0.3 mL) and TFA (0.1 mL). The mixture was stirred at 25° C. for 2 h. The reaction mixture was concentrated under reduced pressure to give 5-(azetidin-3-yl)-3-methylpyrimidin-4(3H)-one (100 mg, crude) was obtained as a yellow oil.

### Synthesis of 1-(azetidin-3-yl)-3-methoxy-1H-pyrazole

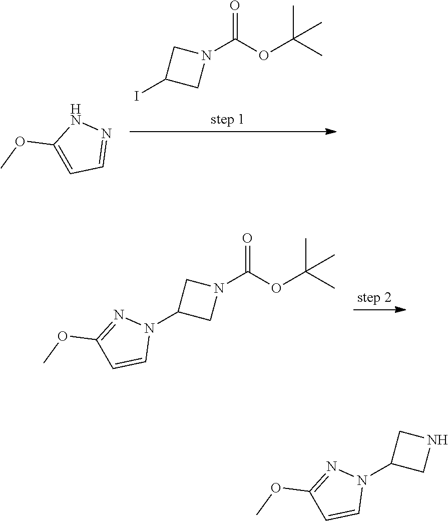

### Step 1: Preparation of Tert-Butyl 3-(3-methoxy-1H-pyrazol-1-yl)azetidine-1-carboxylate

To a solution of 5-methoxy-1H-pyrazole (0.10 mg, 1.0 mmol, 1 eq) in dimethylformamide (1 mL) was added Cs2CO3 (0.49 g, 1.5 mmol, 1.50 eq), the mixture was stirred at 25° C. for 10 min, then tert-butyl 3-iodoazetidine-1-carboxylate (0.28 g, 1.01 mmol, 1 eq) was added. After stirring for 1 h, the yellow reaction mixture was diluted with water (10 mL) and extracted with EtOAc (10 mL×2). The combined organic layers were washed with saturated brine (10 mL×2), dried over Na2SO4, filtered and concentrated under reduced pressure to give a residue. The residue was purified by prep-TLC to give tert-butyl 3-(3-methoxy-1H-pyrazol-1-yl)azetidine-1-carboxylate (20 mg, 79 μmol, 25% yield). LCMS (ESI) m/z=254.2 [M+H]+. 1H NMR (400 MHz, CDCl3) 7.28 (d, J=2.4 Hz, 1H), 5.69 (d, J=2.4 Hz, 1H), 4.90-4.79 (m, 1H), 4.31 (s, 2H), 4.29 (d, J=1.6 Hz, 2H), 3.89 (s, 3H), 1.46 (s, 9H).

### Step 2: Preparation of 1-(azetidin-3-yl)-3-methoxy-1H-pyrazole

To a solution of tert-butyl 3-(3-methoxy-1H-pyrazol-1-yl)azetidine-1-carboxylate (20 mg, 79 μmol, 1.0 eq) in DCM (0.3 mL) and TFA (0.1 mL). The mixture was stirred at 25° C. for 1 h. The resulting yellow solution was concentrated under reduced pressure to give 1-(azetidin-3-yl)-3-methoxy-1H-pyrazole (12 mg, crude) as a yellow oil.

### Synthesis of tert-butyl 3-(4-methylpyridin-3-yl)azetidine-1-carboxylate

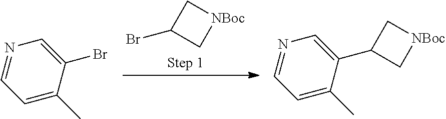

### Step 1: Preparation of Tert-Butyl 3-(4-methylpyridin-3-yl)azetidine-1-carboxylate

To a 15 mL vial equipped with a stir bar was added 3-bromo-4-methylpyridine (3.0 g, 1.0 eq), tert-butyl 3-bromoazetidine-1-carboxylate (8.2 g, 2.0 eq), Ir[dF(CF₃)ppy]₂(dtbpy)(PF₆) (0.22 g, 0.010 eq), NiCl₂.dtbbpy (0.10 g, 0.015 eq), TTMSS (4.3 g, 1.0 eq), Na₂CO₃ (3.7 g, 2.0 eq) was added in DME (0.17 L). The vial was sealed and placed under nitrogen. The reaction was stirred and irradiated with a 10 W blue LED lamp, with cooling water to keep the reaction temperature at 25° C. for 14 h to give a yellow solution. The reaction mixture was concentrated under reduced pressure to give a residue. The residue was purified by flash silica gel chromatography to give tert-butyl 3-(4-methylpyridin-3-yl)azetidine-1-carboxylate (2.9 g, 69% yield) as a yellow oil.

The following intermediates in Table 84 were prepared using the method described above in step 1 for the preparation of tert-butyl 3-(4-methylpyridin-3-yl)azetidine-1-carboxylate and utilizing the appropriate starting materials and modifications.

TABLE 84

| Name | Structure | LCMS | H-NMR data |
|---|---|---|---|
| tert-butyl 3-(4- methoxypyridin-3- yl)azetidine-1- carboxylate | 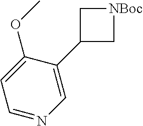 [SMILES: COc1ccncc1C1CN(C(=O)OC(C)(C)C)C1] | 265.0 [M + H]⁺ | 1H NMR (400 MHz, CD₃OD) δ 8.40-8.30 (m, 2 H), 7.08 (d, J = 5.6 Hz, 1 H), 4.32-4.23 (m, 2 H), 4.09 (t, J = 7.6 Hz, 2 H), 3.94 (s, 3 H), 3.89 (s, 1 H), 1.46 (s, 9 H) |
| tert-butyl 3-(5- cyano-1-methyl- 1H-pyrazol-4- yl)azetidine-1- carboxylate | 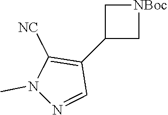 [SMILES: Cn1ncc(C2CN(C(=O)OC(C)(C)C)C2)c1C#N] | 285.1 [M + H]⁺ | ¹H NMR (400 MHz, CD₃OD) 7.66 (s, 1H), 4.34 (t, J = 8.8 Hz, 2H), 4.01 (s, 3H), 4.00-3.94 (m, 2H), 3.90- 3.82 (m, 1H), 1.46 (s, 9H) |
| tert-butyl 3-(3- cyano-1-methyl- 1H-pyrazol-4- yl)azetidine-1- carboxylate | 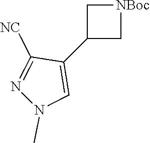 [SMILES: Cn1cc(C2CN(C(=O)OC(C)(C)C)C2)c(C#N)n1] | 263.1 [M + H]⁺ | |
| tert-butyl 3-(4- cyano-2- methylphenyl) azetidine-1- carboxylate | 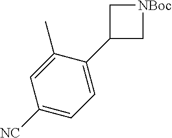 [SMILES: Cc1cc(C#N)ccc1C1CN(C(=O)OC(C)(C)C)C1] | 273.1 [M + H]⁺ | ¹H NMR (400 MHz, CDCl₃) δ 7.61- 7.51 (m, 1H), 7.49-7.36 (m, 2H), 4.39-4.28 (m, 2H), 4.03-3.91 (m, 3H), 2.25 (s, 3H), 1.45 (s, 9H) |
| tert-butyl 3-(5- cyano-3- methylpyridin-2- yl)azetidine-1- carboxylate | 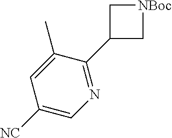 [SMILES: Cc1cc(C#N)cnc1C1CN(C(=O)OC(C)(C)C)C1] | 218.0 [M + H]⁺ | ¹H NMR (400 MHz, CD₃OD) δ 8.76 (d, J = 1.8 Hz, 1H), 7.93 (d, J = 0.9 Hz, 1H), 4.23-4.18 (m, 2H), 4.13-3.99 (m, 2H), 3.63-3.54 (m, 1H), 2.31 (s, 3H), 1.45 (s, 9H) |
| tert-butyl 3-(4- ethylpyridin-3- yl)azetidine-1- carboxylate | 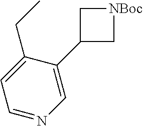 [SMILES: CCc1ccncc1C1CN(C(=O)OC(C)(C)C)C1] | 263.2 [M + H]⁺ | ¹H NMR (400 MHz, CDCl₃) δ 8.43 (s, 1H), 8.25 (d, J = 5.2 Hz, 1H), 6.94 (d, J = 4.8 Hz, 1H), 4.20-4.13 (m, 2H), 3.92-3.87 (m, 2H), 3.85-3.76 (m, 1H), 2.41-2.34 (m, 2H), 1.28 (s, 9H), 1.03 (t, J = 7.6 Hz, 3H) |
| tert-butyl 3-(5- fluoro-4- methylpyridin-3- yl)azetidine-1- carboxylate | 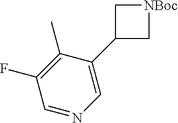 [SMILES: Cc1c(F)cncc1C1CN(C(=O)OC(C)(C)C)C1] | 267.2 [M + H]⁺ | |
| tert-butyl 3-(2- fluoro-4- methylpyridin-3- yl)azetidine-1- carboxylate | 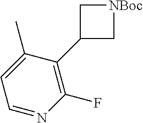 [SMILES: Cc1ccnc(F)c1C1CN(C(=O)OC(C)(C)C)C1] | 267.1 [M + H]⁺ | ¹H NMR (400 MHz, CDCl₃) δ 7.93- 7.99 (m, 1 H) 6.94-6.99 (m, 1 H) 4.18-4.32 (m, 5 H) 2.34 (s, 3 H) 1.44 (d, J = 4.0 Hz, 9 H) |
| tert-butyl 3-(5- methoxy-4- methylpyridin-3- yl)azetidine-1- carboxylate | 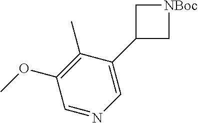 [SMILES: COc1cncc(C2CN(C(=O)OC(C)(C)C)C2)c1C] | 279.0 [M + H]⁺ | 1H NMR (400 MHz, CD₃OD) δ 8.21-8.12 (m, 2H), 4.38 (t, J = 8.0 Hz, 2H), 4.13-4.08 (m, 2H), 3.95 (s, 3H), 3.64-3.55 (m, 1H), 2.15 (s, 3H), 1.47-1.43 (m, 9H) |
| tert-butyl 3-(6- isopropyl-4- methylpyridin-3- yl)azetidine-1- carboxylate | 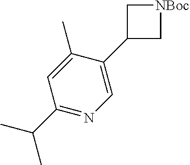 [SMILES: Cc1cc(C(C)C)ncc1C1CN(C(=O)OC(C)(C)C)C1] | 292.1 [M + H]⁺ | ¹H NMR (400 MHz, CDCl₃) δ 8.18 (s, 1H), 8.15-8.07 (m, 1H), 5.51- 5.30 (m, 2H), 5.08-4.95 (m, 1H), 4.87 (d, J = 5.6 Hz, 2H), 4.14 (s, 3H), 3.91-3.76 (m, 1H), 1.29-1.23 (m, 15H) |
| tert-butyl 3-(5- cyano-4- methylpyridin-3- yl)azetidine-1- carboxylate | 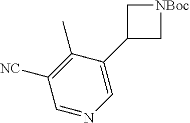 [SMILES: Cc1c(C#N)cncc1C1CN(C(=O)OC(C)(C)C)C1] | 273.3 [M + H]⁺ | |
| tert-butyl 3-(2- cyanopyrimidin-4- yl)azetidine-1- carboxylate | 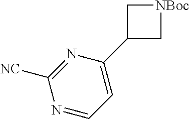 [SMILES: CC(C)(C)OC(=O)N1CC(c2ccnc(C#N)n2)C1] | 282.9 [M + H]⁺ | ¹H NMR (400 MHz, CDCl₃) δ 8.72 (d J = 5.2 Hz, 1H), 7.34 (d, J = 5.2 Hz, 1H), 4.94-4.65 (m, 1H), 4.59- 4.26 (m, 2H), 4.23-4.14 (m, 1H), 4.00-3.88 (m, 1H), 1.42 (s, 9H). |
| tert-butyl 3-(4- methylpyrimidin-5- yl)azetidine-1- carboxylate | 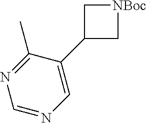 [SMILES: Cc1ncncc1C1CN(C(=O)OC(C)(C)C)C1] | 250.1 [M + H]⁺ | ¹H NMR (400 MHz, CD₃OD) δ 8.90 (s, 1H), 8.70 (s, 1H), 4.42- 4.34 (m, 2H), 4.12-4.05 (m, 3H), 2.47 (s, 3H), 1.46 (s, 9H) |
| tert-butyl 3-(5- phenylpyridin-3- yl)azetidine-1- carboxylate | 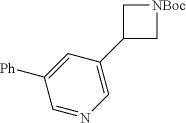 [SMILES: CC(C)(C)OC(=O)N1CC(c2cncc(-c3ccccc3)c2)C1] | 311.2 [M + H]⁺ | |
| tert-butyl 3-(4- cyanopyridin-3- yl)azetidine-1- carboxylate | 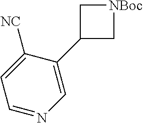 [SMILES: CC(C)(C)OC(=O)N1CC(c2cnccc2C#N)C1] | 260.1 [M + H]⁺ | ¹H NMR (400 MHz, CDCl₃) δ 8.88- 8.98 (m, 1H) 8.69-8.76 (m, 1H) 7.51 (d, J = 4.88 Hz, 1H) 4.44-4.53 (m, 2H), 4.09-4.18 (m, 2H) 3.40 (s, 1H) 1.40-1.57 (m, 9H) |
| tert-butyl 3-(6- cyanopyridin-2- yl)azetidine-1- carboxylate | 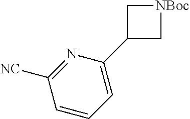 [SMILES: CC(C)(C)OC(=O)N1CC(c2cccc(C#N)n2)C1] | 282.2 [M + H]⁺ | ¹H NMR (400 MHz, CD₃OD) δ 7.97-7.91 (m, 1H), 7.77-7.74 (m, 1H), 7.60-7.58 (m, 1H), 4.36- 4.25 (m, 2H), 4.13 (t, J = 6.4 Hz, 2H), 4.05-3.99 (m, 1H), 1.49-1.46 (m, 9H) |
| tert-butyl 3-(3- fluoropyrazin-2- yl)azetidine-1- carboxylate | 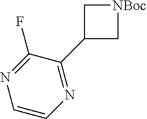 [SMILES: CC(C)(C)OC(=O)N1CC(c2nccnc2F)C1] | 254.3 [M + H]⁺ | ¹H NMR (400 MHz, CDCl₃) δ 8.53- 8.49 (m, 1H), 8.12 (t, J = 2.0 Hz, 1H), 4.35-4.30 (m, 2H), 4.29-4.25 (m, 2H), 4.16-4.08 (m, 1H), 1.47 (s, 9H) |
| tert-butyl 3-(5- fluoroquinolin-6- yl)azetidine-1- carboxylate | 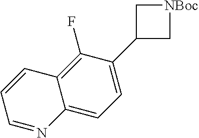 [SMILES: CC(C)(C)OC(=O)N1CC(c2ccc3ncccc3c2F)C1] | 303.3 [M + H]⁺ | ¹H NMR (400 MHz, CD₃OD) δ 8.94-8.87 (m, 1H), 8.61-8.54 (m, 1H), 7.95-7.85 (m, 2H), 7.59 (m, 1H), 4.48-4.41 (m, 2H), 4.34-4.25 (m, 1H), 4.17-4.10 (m, 2H), 1.49 (s, 9H) |
| tert-butyl 3-(4- fluoro-6- methoxypyridin-3- yl)azetidine-1- carboxylate | 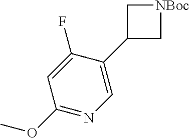 [SMILES: COc1cc(F)c(C2CN(C(=O)OC(C)(C)C)C2)cn1] | 283.2 [M + H]⁺ | ¹H NMR (400 MHz, CD₃OD) δ 8.18-8.05 (m, 1H), 6.65-6.51 (m, 1H), 4.35-4.28 (m, 2H), 4.05 (t, J = 7.6 Hz, 2H), 3.97-3.92 (m, 1H), 3.91 (s, 3H), 1.46 (s, 9H). |
| tert-butyl 3-(7- fluoroquinolin-6- yl)azetidine-1- carboxylate | 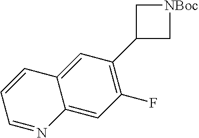 [SMILES: CC(C)(C)OC(=O)N1CC(c2cc3cccnc3cc2F)C1] | 303.2 [M + H]⁺ | |
| tert-butyl 3-(2- (difluoromethyl) pyridin-3-yl)azetidine- 1-carboxylate | 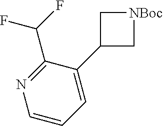 [SMILES: CC(C)(C)OC(=O)N1CC(c2cccnc2C(F)F)C1] | 285.1 [M + H]⁺ | ¹H NMR (400 MHz, CDCl₃) δ 8.53 (d, J = 4.4 Hz, 1H), 8.07 (d, J = 7.6 Hz, 1H), 7.50 (d, J = 4.8, 7.8 Hz, 1H), 6.68 (t, J = 54.4 Hz, 1H), 4.43-4.29 (m, 3H), 3.96 (s, 2H), 1.47 (s, 9H) |
| tert-butyl 3-(2- fluorophenyl)azetidine- 1-carboxylate | 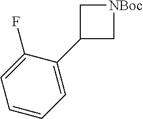 [SMILES: CC(C)(C)OC(=O)N1CC(c2ccccc2F)C1] | 196.0 [M + H]⁺ | ¹H NMR (400 MHz, CD₃OD) δ 7.40 (dt, J = 1.8, 7.8 Hz, 1H), 7.32- 7.25 (m, 1H), 7.22-7.16 (m, 1H), 7.09 (ddd, J = 1.2, 8.2, 10.8 Hz, 1H), 4.38-4.26 (m, 2H), 4.08-3.98 (m, 3H), 1.46 (s, 9H) |
| tert-butyl 3-(6- fluoro-4- methoxypyridin-3- yl)azetidine-1- carboxylate | 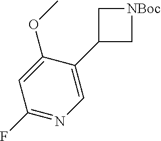 [SMILES: COc1cc(F)ncc1C1CN(C(=O)OC(C)(C)C)C1] | 283.1 [M + H]⁺ | ¹H NMR (400 MHz, CDCl₃) δ 7.92 (s, 1H), 6.40 (d, J = 3.6 Hz, 1H), 4.09- 4.01 (m, 4H), 3.90 (s, 3H), 3.62- 3.59 (m, 1H), 1.47 (s, 9H). |
| tert-butyl 3-(2- fluoro-4- methoxypyridin-3- yl)azetidine-1- carboxylate | 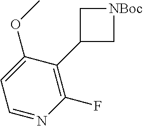 [SMILES: COc1ccnc(F)c1C1CN(C(=O)OC(C)(C)C)C1] | 283.1 [M + H]⁺ | |
| tert-butyl 3-(5- fluoropyrimidin-4- yl)azetidine-1- carboxylate | 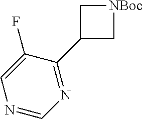 [SMILES: CC(C)(C)OC(=O)N1CC(c2ncncc2F)C1] | 254.3 [M + H]⁺ | ¹H NMR (400 MHz, CD₃OD) δ 9.02 (d, J = 2.8 Hz, 1H), 8.74-8.61 (m, 1H), 4.31-4.23 (m, 5H), 1.46 (s, 9H). |
| tert-butyl 3-(1- methyl-4-oxo-1,4- dihydropyridin-3- yl)azetidine-1- carboxylate | 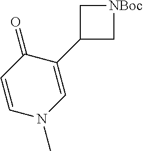 [SMILES: Cn1ccc(=O)c(C2CN(C(=O)OC(C)(C)C)C2)c1] | 265.2 [M + H]⁺ | |
| tert-butyl 3-(5- fluoropyridin-3- yl)azetidine-1- carboxylate | 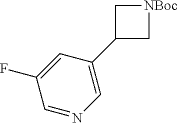 [SMILES: CC(C)(C)OC(=O)N1CC(c2cncc(F)c2)C1] | 253.1 [M + H]⁺ | ¹H NMR (400 MHz, CDCl₃) δ 8.61- 8.43 (m, 2H), 7.82 (d, J = 8.0 Hz, 1H), 4.45 (t, J = 8.4 Hz, 2H), 3.96 (dd, J = 5.6, 8.8 Hz, 2H), 3.92-3.81 (m, 1H), 1.49 (s, 9H) |
| tert-butyl 3-(3- fluoropyridin-4- yl)azetidine-1- carboxylate | 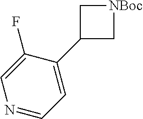 [SMILES: CC(C)(C)OC(=O)N1CC(c2ccncc2F)C1] | 253.4 [M + H]⁺ | ¹H NMR (400 MHz, CDCl₃) δ 8.43- 8.42 (m, 2H), 7.35 (t, J = 6.0 Hz, 1H), 4.38-4.32 (m, 2H), 4.04-3.98 (m, 3H), 1.46 (s, 9H) |
| tert-butyl 3-(2- hydroxypyridin-4- yl)azetidine-1- carboxylate | 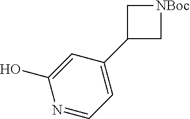 [SMILES: CC(C)(C)OC(=O)N1CC(c2ccnc(O)c2)C1] | 251.1 [M + H]⁺ | ¹H NMR (400 MHz, CDCl₃) δ 7.39 (d, J = 6.4 Hz, 1H), 6.47 (s, 1H), 6.33 (d, J = 6.4 Hz, 1H), 4.29 (t, J = 8.8 Hz, 2H), 4.12 (s, 1H), 3.76 (s, 1H), 3.58 (s, 1H), 1.47 (s, 9H) |
| tert-butyl 3-(4- (difluoromethoxy) pyridin-3- yl)azetidine-1- carboxylate | 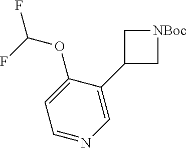 [SMILES: CC(C)(C)OC(=O)N1CC(c2cnccc2OC(F)F)C1] | 301.4 [M + H]⁺ | ¹H NMR (400 MHz, CDCl₃) 8.77 (s, 1H), 8.71 (d, J = 6.4 Hz, 1H), 7.41 (d, J = 6.4 Hz, 1H), 6.84-6.63 (m, 1H), 5.31 (s, 1H), 4.47-4.35 (m, 2H), 4.11-4.06 (m, 2H), 1.47 (s, 9H) |
| tert-butyl 3-(5- fluoro-1-methyl- 1H-pyrazol-4- yl)azetidine-1- carboxylate | 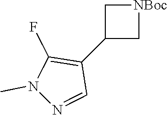 [SMILES: Cn1ncc(C2CN(C(=O)OC(C)(C)C)C2)c1F] | 256.3 [M + H]⁺ | ¹H NMR (400 MHz, CDCl₃) δ 7.35 (s, 1H), 4.25 (t, J = 8.8 Hz, 2H), 3.92 (dd, J = 6.5, 8.1 Hz, 2H), 3.72 (s, 3H), 3.64-3.54 (m, 1H), 1.46 (s, 9H) |
| tert-butyl 3-(3- methylpyrazin-2- yl)azetidine-1- carboxylate | 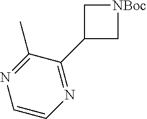 [SMILES: Cc1nccnc1C1CN(C(=O)OC(C)(C)C)C1] | 250.2 [M + H]⁺ | ¹H NMR (400 MHz, CD₃OD) δ 8.29 (d, J = 2.4 Hz, 1H), 8.15 (d, J = 2.4 Hz, 1H), 4.11-4.01 (m, 5H), 2.31 (s, 3H), 1.27 (s, 9H) |
| tert-butyl 3-(5- methylpyrimidin-4- yl)azetidine-1- carboxylate | 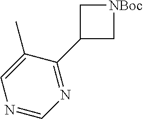 [SMILES: Cc1cncnc1C1CN(C(=O)OC(C)(C)C)C1] | 250.1 [M + H]⁺ | ¹H NMR (400 MHz, DMSO) δ 9.02 (s, 1H), 8.54 (s, 1H), 4.10 (d, J = 4.8 Hz, 5H), 2.17 (s, 3H), 1.39 (s, 9H) |
| tert-butyl 3-(5- cyanopyridin-3- yl)azetidine-1- carboxylate | 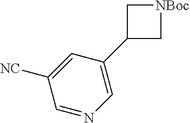 [SMILES: CC(C)(C)OC(=O)N1CC(c2cncc(C#N)c2)C1] | 259.8 [M + H]⁺ | ¹H NMR (400 MHz, CDCl₃) δ 9.06 (s, 1H), 9.03 (s, 1H), 8.87-8.83 (m, 1H), 3.74-3.69 (m, 1H), 3.65-3.59 (m, 2H), 3.55-3.46 (m, 2H), 1.26 (s, 9H) |
| tert-butyl 3-(4- ethoxypyridin-3- yl)azetidine-1- carboxylate | 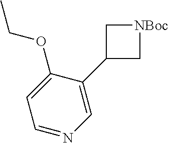 [SMILES: CCOc1ccncc1C1CN(C(=O)OC(C)(C)C)C1] | | ¹H NMR (400 MHz, CDCl₃) δ 8.52- 8.30 (m, 2H), 6.97-6.80 (m, 1H), 4.35-4.24 (m, 2H), 4.22-4.06 (m, 4H), 4.02-3.85 (m, 1H), 1.55-1.40 (m, 12H). |
| tert-butyl 4- (pyrimidin-4- yl)piperidine-1- carboxylate | 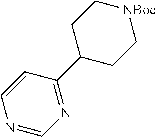 [SMILES: CC(C)(C)OC(=O)N1CCC(c2ccncn2)CC1] | 264.1 [M + H]⁺ | ¹H NMR (400 MHz, CDCl₃) δ 9.20- 9.13 (m, 1H), 8.66 (d, J = 5.2 Hz, 1H), 7.19 (dd, J = 1.2, 5.2 Hz, 1H), 3.80-3.61 (m, 1H), 3.50-3.34 (m, 2H), 2.88-2.80 (m, 2H), 1.96-1.90 (m, 2H), 1.71 (dq, J = 4.4, 12.4 Hz, 2H), 1.48 (s, 9H) |
| tert-butyl 4-(5- fluoropyrimidin-4- yl)piperidine-1- carboxylate | 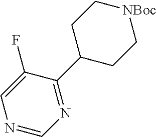 [SMILES: CC(C)(C)OC(=O)N1CCC(c2ncncc2F)CC1] | 282.3 [M + H]⁺ | ¹H NMR (400 MHz, CDCl₃) 8.96 (d, J = 2.8 Hz, 1H), 8.49 (d, J = 2.0 Hz, 1H), 3.41 (d, J = 11.2 Hz, 1H), 3.26-3.14 (m, 1H), 2.87 (t, J = 11.6 Hz, 2H), 2.69-2.60 (m, 1H), 1.94- 1.85 (m, 2H), 1.83 (s, 2H), 1.49 (s, 9H). |

### Synthesis of 2-(3-(methoxymethyl)azetidin-3-yl)pyrimidine

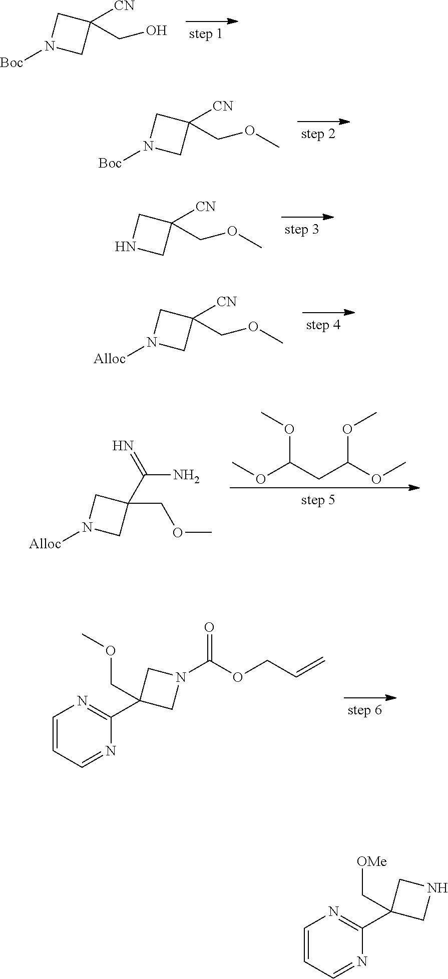

### Step 1: Preparation of Tert-Butyl 3-cyano-3-(methoxymethyl)azetidine-1-carboxylate

To a cooled (0° C.) solution of tert-butyl 3-cyano-3-(hydroxymethyl)azetidine-1-carboxylate (2.5 g, 12 mmol, 1 eq) in THF (200 mL) was added NaH (0.36 g, 15 mmol, 1.3 eq). The mixture was stirred at 25° C. for 30 min, followed by addition of iodomethane (3.3 g, 24 mmol, 2 eq). The mixture was stirred at 25° C. for 1 h to give a gray suspension. The reaction mixture was quenched by H2O (10 mL) at 0° C., subsequently diluted with water (20 mL) and extracted with EtOAc (100 mL×2). The combined organic layers were washed with saturated brine (100 mL×2), dried over Na₂SO₄, filtered, and concentrated under reduced pressure to give a residue. The residue was purified by column chromatography to give tert-butyl 3-cyano-3-(methoxymethyl)azetidine-1-carboxylate (1 g, 4.4 mmol, 38% yield) as a white oil. 1H NMR (400 MHz, CDCl3) δ 4.14 (d, J=8.8 Hz, 2H), 3.88 (d, J=8.8 Hz, 2H), 3.60 (s, 2H), 3.40 (s, 3H), 1.38 (s, 9H).

### Step 2: Preparation of 3-(methoxymethyl)azetidine-3-carbonitrile

A solution of tert-butyl 3-cyano-3-(methoxymethyl)azetidine-1-carboxylate (1 g, 1.7 mmol, 1.0 eq) in methylene chloride (6 mL) and TFA (3 mL) was stirred at 25° C. for 30 min to give a yellow solution. The reaction mixture was concentrated under reduced pressure to give 3-(methoxymethyl)azetidine-3-carbonitrile (1 g, crude) as a clear oil.

### Step 3: Preparation of Allyl 3-cyano-3-(methoxymethyl)azetidine-1-carboxylate

To a solution of 3-(methoxymethyl)azetidine-3-carbonitrile (1 g, 7.9 mmol, 1.0 eq) in methylene chloride (1 mL) was added triethylamine (3.2 g, 32 mmol, 4 eq), followed by dropwise addition of AllocCl (0.95 g, 7.9 mmol, 1 eq) at 0° C. The mixture was stirred at 25° C. for 3 h to give a yellow solution. The reaction mixture was concentrated under reduced pressure to give a residue. The residue was purified by column chromatography to afford allyl 3-cyano-3-(methoxymethyl)azetidine-1-carboxylate (0.85 g, 4.0 mmol, 51% yield) as a yellow oil. 1H NMR (400 MHz, CDCl3) δ 5.97-5.85 (m, 1H), 5.35-5.28 (m, 1H), 5.28-5.24 (m, 1H), 4.60 (d, J=6.0 Hz, 2H), 4.31 (d, J=9.2 Hz, 2H), 4.08 (d, J=8.8 Hz, 2H), 3.68 (s, 2H), 3.48 (s, 3H).

### Step 4: Preparation of Allyl 3-carbamimidoyl-3-(methoxymethyl)azetidine-1-carboxylate

Acetyl chloride (9.0 g, 0.14 mol, 30 eq) was slowly added to a cooled (0° C.) mixture of EtOH (6 mL) and CH3Cl (8 mL). The mixture was stirred for 2 h, followed by slow addition of a solution of allyl 3-cyano-3-(methoxymethyl)azetidine-1-carboxylate (0.80 g, 3.8 mmol, 1 eq) in CH₃C₁ (8 mL). The resulting mixture was stirred at 25° C. for 12 h and subsequently concentrated in vacuo to give a residue. The residue was redissolved by EtOH (6 mL), followed by addition of NH₃/MeOH (1.9 g, 0.14 mol, 30 eq, 7 M). The mixture was stirred at 25° C. for 12 h to give a white suspension. The reaction mixture was concentrated under reduced pressure to give allyl 3-carbamimidoyl-3-(methoxymethyl)azetidine-1-carboxylate (0.8 g, crude) as a white oil. LCMS (ESI) m/z=228.1 [M+H]+.

### Step 5: Preparation of Allyl 3-(methoxymethyl)-3-(pyrimidin-2-yl)azetidine-1-carboxylate

To a solution of allyl 3-carbamimidoyl-3-(methoxymethyl)azetidine-1-carboxylate (0.8 g, 3.5 mmol, 1 eq) in pyridine (10 mL) was added 1,1,3,3-tetramethoxypropane (1.2 g, 7.0 mmol, 2 eq). The mixture was heated to 170° C. under MW for 1 h to give a black solution. The reaction mixture was concentrated under reduced pressure to give allyl 3-(methoxymethyl)-3-(pyrimidin-2-yl)azetidine-1-carboxylate (0.4 g, 1.52 mmol, 43% yield) as a black oil. 1H NMR (400 MHz, CDCl3) δ 8.76 (d, J=4.8 Hz, 2H), 7.22 (t, J=4.8 Hz, 1H), 6.00-5.89 (m, 1H), 5.37-5.29 (m, 1H), 5.25-5.19 (m, 1H), 4.59-4.57 (m, 2H), 4.20 (d, J=8.8 Hz, 2H), 3.90 (s, 2H), 3.82 (s, 2H), 3.37 (s, 3H).

### Step 6: Preparation of 2-(3-(methoxymethyl)azetidin-3-yl)pyrimidine

To a solution of allyl 3-(methoxymethyl)-3-(pyrimidin-2-yl)azetidine-1-carboxylate (0.4 g, 1.5 mmol, 1 eq) in methylene chloride (5 mL) was added pyrrolidine (0.21 g, 3.0 mmol, 2 eq) and Pd(PPh₃)₄(0.17 g, 0.15 mmol, 0.1 eq) under N2, the mixture was stirred at 25° C. for 1 h to give a yellow solution. The reaction mixture was concentrated under reduced pressure to give a residue. The residue was dissolved in H2O (3 mL) and purified by reversed phase (TFA) to lyophilized to give a residue. The residue was re-purification by prep-HPLC (column: Phenomenex Luna C18 150×25 mm×10 um, mobile phase: water (TFA)-ACN; B %: 13%-43%, 11.5 min) to lyophilized to give 2-(3-(methoxymethyl)azetidin-3-yl)pyrimidine (70 mg, 0.39 mmol, 26% yield) as a yellow oil. 1H NMR (400 MHz, CDCl3) δ 8.78-8.71 (m, 2H), 7.26 (s, 1H), 4.56 (s, 2H), 4.33 (s, 2H), 3.86 (s, 2H), 3.47 (s, 3H).

### Synthesis of 3-(azetidin-3-yl)pyridin-4-ol

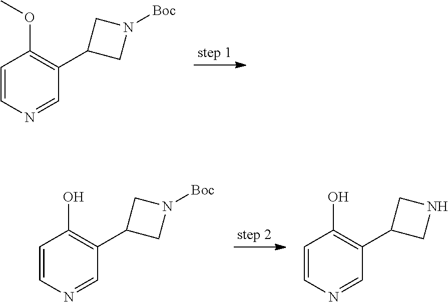 [SMILES: COc1ccncc1C1CN(C(=O)OC(C)(C)C)C1 ; CC(C)(C)OC(=O)N1CC(c2cnccc2O)C1 ; Oc1ccncc1C1CNC1]

### Step 1: Preparation of Tert-Butyl 3-(4-hydroxypyridin-3-yl)azetidine-1-carboxylate

To a solution of tert-butyl 3-(4-methoxypyridin-3-yl)azetidine-1-carboxylate (0.3 g, 1.1 mmol, 1 eq) in dimethylformamide (3.0 mL) was added NaOEt (0.19 g, 2.3 mmol, 2 eq). The mixture was stirred at 100° C. for 2 h. The resulting yellow solution was cooled to room temperature and diluted with water (50 mL) and extracted with EtOAc (50 mL×2). To the aqueous phase was added saturated NaHCO₃ (to adjust the pH=7-8) and extracted with EtOAc (50 mL×2). The combined organic layers were washed with saturated brine (40 mL×2), dried over Na₂SO₄, filtered and concentrated under reduced pressure to give tert-butyl 3-(4-hydroxypyridin-3-yl)azetidine-1-carboxylate (0.1 g, crude) as a white solid. LCMS (ESI) m/z=251.1 [M+H]+.

### Step 2: Preparation of 3-(azetidin-3-yl)pyridin-4-ol

To a solution of tert-butyl 3-(4-hydroxypyridin-3-yl)azetidine-1-carboxylate (90 mg, 0.36 mmol, 1 eq) in methylene chloride (1.0 mL) was added TFA (0.3 mL). The mixture was stirred at 25° C. for 15 min to give yellow solution. The reaction mixture was concentrated under reduced pressure to give 3-(azetidin-3-yl)pyridin-4-ol (90 mg, crude) as a yellow oil which was used to next step without any purification. LCMS (ESI) m/z=151.2 [M+H]+.

### Synthesis of (R)-1-(1-methyl-1H-pyrazol-3-yl)pyrrolidin-3-amine

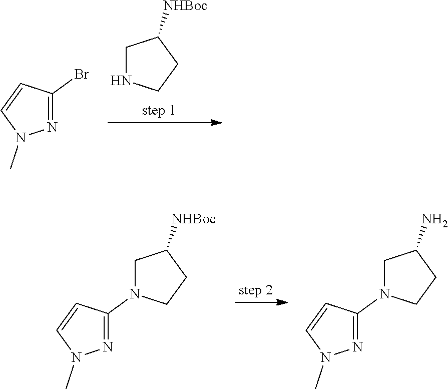

### Step 1: Preparation of Tert-Butyl (R)-(1-(1-methyl-1H-pyrazol-3-yl)pyrrolidin-3-yl)carbamate

A mixture of (R)-tert-butyl pyrrolidin-3-ylcarbamate tert-butyl N-[(3R)-pyrrolidin-3-yl]carbamate (0.80 mg, 4.3 mmol, 2 eq), 3-bromo-1-methyl-1H-pyrazole (0.35 g, 2.16 mmol, 1 eq), CuI (0.13 g, 0.65 mmol, 0.3 eq), L-Proline (74.6 mg, 648 μmol, 0.3 eq) and K2CO3 (0.89 g, 6.5 mmol, 3 eq) in DMSO (4 mL) was degassed and purged with N2 (3 times). The reaction mixture was stirred at 100° C. for 12 h under N2 atmosphere. The mixture was diluted with water (10 mL) and extracted with EtOAc (20 mL×3). The combined organic layers were washed with brine (20 mL×3), dried over anhydrous Na2SO4, filtered, and concentrated in vacuo to give a residue. The residue was purified by flash silica gel chromatography to give tert-butyl (R)-(1-(1-methyl-1H-pyrazol-3-yl)pyrrolidin-3-yl)carbamate (0.67 g, 2.5 mmol, 50% yield) as a yellow oil. 1H NMR (400 MHz, CD3OD) δ 7.28 (d, J=2.0 Hz, 1H), 5.49 (d, J=2.0 Hz, 1H), 4.21-4.12 (m, 1H), 3.69 (s, 3H), 3.52-3.45 (m, 1H), 3.40-3.33 (m, 2H), 3.29-3.20 (m, 1H), 3.10-3.03 (m, 1H), 2.25-2.17 (m, 1H), 1.45 (s, 9H).

The following intermediates in Table 85 were prepared using the method described above in step 1 for the preparation of tert-butyl (R)-(1-(1-methyl-1H-pyrazol-3-yl)pyrrolidin-3-yl)carbamate and utilizing the appropriate starting materials and modifications.

TABLE 85

| Name | Structure | LCMS |
|---|---|---|
| tert-butyl (R)-(1-(1-methyl- 1H-pyrazol-5-yl)pyrrolidin-3- yl)carbamate | 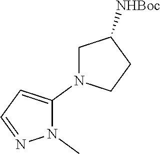 [SMILES: Cn1nccc1N1CC[C@@H](NC(=O)OC(C)(C)C)C1] | 267.1 [M + H]⁺ |
| tert-butyl (R)-(1-(3- methylpyridin-2-yl) pyrrolidin-3- yl)carbamate | 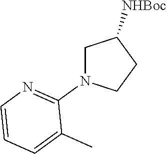 [SMILES: Cc1cccnc1N1CC[C@@H](NC(=O)OC(C)(C)C)C1] | 278.0 [M + H]⁺ |
| tert-butyl ((3S,4S)-4-fluoro- 1-(3-methylpyridin-2-yl) pyrrolidin- 3-yl)carbamate | 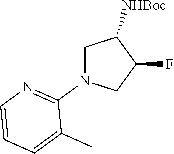 [SMILES: Cc1cccnc1N1C[C@H](NC(=O)OC(C)(C)C)[C@@H](F)C1] | 296.0 [M + H]⁺ |

### Step 2: Preparation of (R)-1-(1-methyl-1H-pyrazol-3-yl)pyrrolidin-3-amine

To a solution of tert-butyl (R)-(1-(1-methyl-1H-pyrazol-3-yl)pyrrolidin-3-yl)carbamate (300 mg, 1.12 mmol, 1 eq) in DCM (3 mL) was added TFA (1 mL). The mixture was stirred at 25° C. for 0.5 h. The reaction mixture was filtered and concentrated under reduced pressure. The residue was purified by flash silica gel chromatography to give (R)-1-(1-methyl-1H-pyrazol-3-yl)pyrrolidin-3-amine (0.13 g, 0.79 mmol, 70% yield) as a yellow oil. 1H NMR (400 MHz, CD3OD) δ 7.35 (d, J=2.4 Hz, 1H), 5.60 (d, J=2.4 Hz, 1H), 3.99-3.91 (m, 1H), 3.73 (s, 3H), 3.57-3.45 (m, 2H), 3.42-3.36 (m, 1H), 3.28-3.21 (m, 1H), 2.48-2.38 (m, 1H), 2.11-2.02 (m, 1H).

### Synthesis of tert-butyl 3-(2-methoxyethoxy)-3-(pyridin-2-yl)azetidine-1-carboxylate

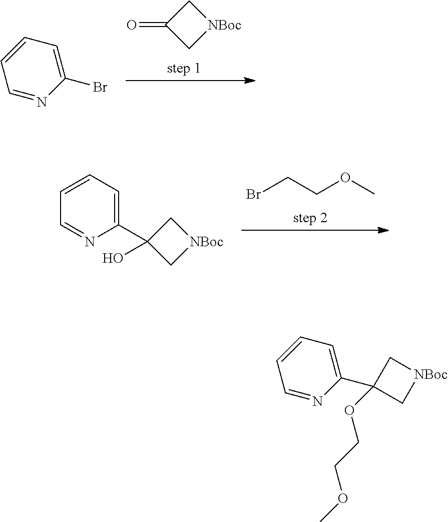

### Step 1: Preparation of Tert-Butyl 3-hydroxy-3-(pyridin-2-yl)azetidine-1-carboxylate

To a cooled (−78° C.) solution of 2-bromopyridine (5 g, 31.6 mmol, 1 eq) and tert-butyl 3-oxoazetidine-1-carboxylate (5.40 g, 31.6 mmol, 1 eq) in THF (50 mL) was added n-BuLi (14 mL, 34.81 mmol, 1.1 eq). After stirring for 2 h, the reaction mixture quenched by H2O (50 mL), then extracted with EtOAc (50 mL×3). The combined organic layers were washed with brine (50 mL×3), dried over anhydrous Na₂SO₄, filtered and concentrated under reduced pressure to give a residue. The residue was purified by column chromatography to give tert-butyl 3-hydroxy-3-(pyridin-2-yl)azetidine-1-carboxylate (2.90 g, 11.5 mmol, 36.7% yield) as a yellow solid. ¹H NMR (400 MHz, CDCl₃) δ 8.73-8.51 (m, 1H), 7.81 (dt, J=1.8, 7.7 Hz, 1H), 7.60 (d, J=7.9 Hz, 1H), 7.31 (ddd, J=1.1, 4.8, 7.5 Hz, 1H), 6.56 (s, 1H), 4.35-4.10 (m, 2H), 3.92 (s, 2H), 1.41 (s, 9H).

### Step 2: Preparation of tert-butyl 3-(2-methoxyethoxy)-3-(pyridin-2-yl)azetidine-1-carboxylate

To a solution of tert-butyl 3-hydroxy-3-(pyridin-2-yl) azetidine-1-carboxylate (500 mg, 1.99 mmol, 1 eq), 1-bromo-2-methoxyethane (414 mg, 2.98 mmol, 1.5 eq), TBAI (735 mg, 1.99 mmol, 1 eq) in DMF (5 mL) added NaH (119 mg, 4.97 mmol, 2.5 eq) at 0° C. The mixture was stirred at 25° C. for 12 h. The reaction mixture was quenched with NH4Cl aq (50 mL) and extracted with EtOAc (50 mL×2), the combined organic layers were washed with saturated brine (40 mL×2), dried over Na2SO4, filtered, and concentrated under reduced pressure to give a residue. The residue was purified by column chromatography to give tert-butyl 3-(2-methoxyethoxy)-3-(pyridin-2-yl)azetidine-1-carboxylate (426 mg, 1.38 mmol, 69.4% yield) as a yellow solid. LCMS (ESI) m/z=308.8 [M+H]+.

The following intermediates in Table 86 were prepared using the method described above in step 2 for the preparation of tert-butyl 3-(2-methoxyethoxy)-3-(pyridin-2-yl)azetidine-1-carboxylate and utilizing the appropriate starting materials and modifications.

TABLE 86

| Name | Structure | LCMS |
|---|---|---|
| tert-butyl 3-(2- methoxyethoxy)- 3-(pyridin-3-yl) azetidine-1- carboxylate | 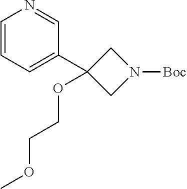 [SMILES: COCCOC1(c2cccnc2)CN(C(=O)OC(C)(C)C)C1] | 309.5 [M + H]⁺ |
| tert-butyl 3-methoxy- 3-(pyridin- 3-yl)azetidine-1- carboxylate | 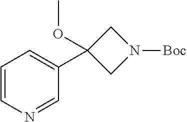 [SMILES: COC1(c2cccnc2)CN(C(=O)OC(C)(C)C)C1] | 265.1 [M + H]⁺ |
| tert-butyl 3- methoxy-3-(4- methylpyridin-3-yl) azetidine-1- carboxylate | 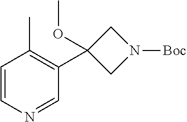 [SMILES: COC1(c2cnccc2C)CN(C(=O)OC(C)(C)C)C1] | 279.1 [M + H]⁺ |
| tert-butyl 3- methoxy-3- (pyrimidin-5-yl) azetidine-1- carboxylate | 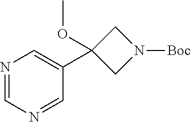 [SMILES: COC1(c2cncnc2)CN(C(=O)OC(C)(C)C)C1] | 266.2 [M + H]⁺ |
| tert-butyl 3- methoxy-3-(4- methylpyrimidin-5- yl)azetidine-1- carboxylate | 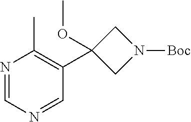 [SMILES: COC1(c2cncnc2C)CN(C(=O)OC(C)(C)C)C1] | 279.8 [M + H]⁺ |

### Synthesis of cyclopropyl (R)-3-aminopyrrolidine-1-carboxylate

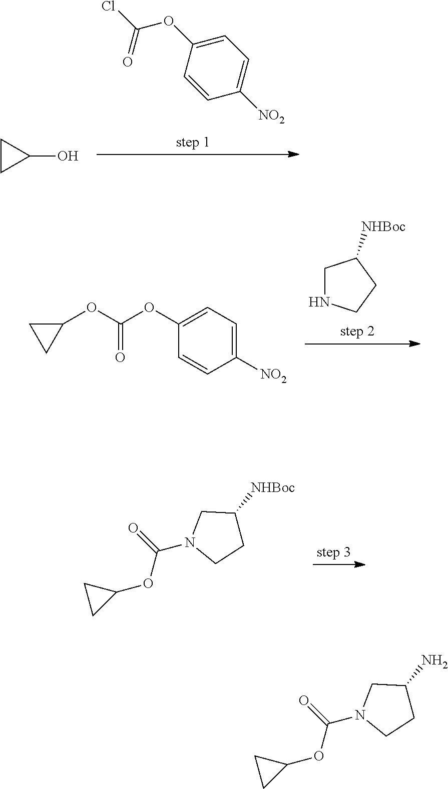

### Step 1: Preparation of cyclopropyl (4-nitrophenyl) carbonate

To a solution of cyclopropanol (0.33 g, 5.8 mmol, 1.2 eq) in DCM (10 mL) was added 4-nitrophenyl carbonochloridate (1 g, 4.9 mmol, 1 eq). After stirring for 2 h, the yellow solution was concentrated under reduced pressure. The residue was purified by flash silica gel chromatography to give cyclopropyl (4-nitrophenyl) carbonate (1 g, 4.48 mmol, 91% yield) as white solid. 1H NMR (400 MHz, CDCl3) δ 8.31-8.24 (m, 2H), 7.42-7.36 (m, 2H), 4.28 (tt, J=3.2, 6.4 Hz, 1H), 0.93-0.87 (m, 2H), 0.86-0.80 (m, 2H).

### Step 2: Preparation of cyclopropyl (R)-3-((tert-butoxycarbonyl)amino)pyrrolidine-1-carboxylate

To a solution of cyclopropyl (4-nitrophenyl) carbonate (0.10 g, 0.45 mmol, 1.3 eq) in DCM (2 mL) was added tert-butyl N-[(3R)-pyrrolidin-3-yl]carbamate (64 mg, 0.34 mmol, 1 eq) and N,N-diisopropylethylamine (44 mg, 0.34 mmol, 1 eq), the mixture was stirred at 25° C. for 30 min to give a yellow clean solution. The reaction mixture was concentrated in vacuo to give cyclopropyl (R)-3-((tert-butoxycarbonyl)amino)pyrrolidine-1-carboxylate (0.10 g, crude) as a yellow solid. LCMS (ESI) m/z=271.5 [M+H]+.

### Step 3: Preparation of cyclopropyl (R)-3-aminopyrrolidine-1-carboxylate

To a solution of cyclopropyl (R)-3-((tert-butoxycarbonyl)amino)pyrrolidine-1-carboxylate (80 mg, 0.29 mmol, 1 eq) in DCM (1 mL) was added TFA (0.10 g, 0.88 mmol, 3 eq), the mixture was stirred at 25° C. for 30 min to give a yellow clean solution, the reaction mixture was concentrated under reduced pressure to give cyclopropyl (R)-3-aminopyrrolidine-1-carboxylate (50 mg, crude) as a white solid. LCMS (ESI) m/z=171.4 [M+H]+.

The following intermediates in Table 87 were prepared using the method described above for the synthesis of cyclopropyl (R)-3-aminopyrrolidine-1-carboxylate and utilizing the appropriate starting materials and modifications.

TABLE 87

| Name | Structure | LCMS |
|---|---|---|
| cyclopropyl 3- aminoazetidine- 1-carboxylate | 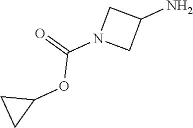 [SMILES: NC1CN(C(=O)OC2CC2)C1] | 157.2 [M + H]⁺ |
| cyclopropyl (3S,4S)- 3-amino-4- fluoropyrrolidine-1- carboxylate | 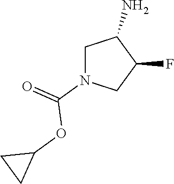 [SMILES: N[C@H]1CN(C(=O)OC2CC2)C[C@@H]1F] | 189.2 [M + H]⁺ |
| (R)-3-amino-N- methylpyrrolidine- 1-carboxamide | 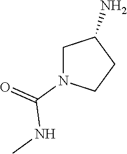 | 144.1 [M + H]⁺ |

### Synthesis of 1′H-spiro[azetidine-3,3′-furo[3,4-c]pyridine]

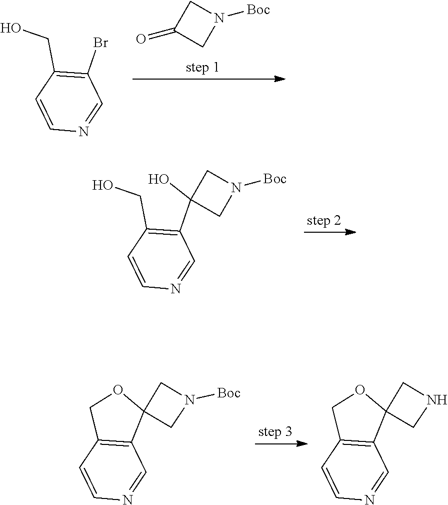

### Step 1: Preparation of Tert-Butyl 3-hydroxy-3-(4-(hydroxymethyl)pyridin-3-yl)azetidine-1-carboxylate

To a cooled (−40° C.) solution of (3-bromopyridin-4-yl)methanol (1 g, 5.3 mmol, 1 eq) in THF (10 mL) was slowly added i-PrMgCl·LiCl (13 mL, 13 mmol, 2.4 eq, 1.0 M in THF). The mixture was stirred at −40° C. for 0.5 h, followed by addition of a solution of tert-butyl 3-oxoazetidine-1-carboxylate (1.1 g, 6.4 mmol, 1.2 eq) in THF (10 mL). The mixture was allowed to warm to ambient temperatures. After 16 h, the reaction mixture was quenched by water (20 mL) and extracted with EtOAc 90 mL (30 mL×3). The combined organic layer was washed with brine 90 mL (30 mL×3), dried over Na₂SO₄, filtered, and concentrated under reduced pressure to give a residue. The residue was purified by column chromatography to give tert-butyl 3-hydroxy-3-(4-(hydroxymethyl)pyridin-3-yl)azetidine-1-carboxylate (0.12 g, 0.43 mmol, 8.1% yield) as a yellow solid. 1H NMR (400 MHz, DMSO-d6) δ 8.56-8.44 (m, 2H), 7.55 (d, J=4.8 Hz, 1H), 5.38 (t, J=5.6 Hz, 1H), 4.53 (d, J=5.6 Hz, 2H), 4.40 (d, J=9.2 Hz, 2H), 4.05-3.92 (m, 2H), 3.31-3.28 (m, 1H), 1.38 (s, 9H).

### Step 2: Preparation of tert-butyl 1′H-spiro[azetidine-3,3′-furo[3,4-c]pyridine]-1-carboxylate

To a cooled (0° C.) solution of tert-butyl 3-hydroxy-3-(4-(hydroxymethyl)pyridin-3-yl)azetidine-1-carboxylate (70 mg, 0.25 mmol, 1 eq) in THF (1 mL) was added sequentially added DIAD (60 mg, 0.30 mmol, 1.2 eq) and PPh3 (78 mg, 0.30 mmol, 1.2 eq). the mixture was stirred at 40° C. for 16 h and subsequently quenched by water (30 mL). The mixture was extracted with EtOAc 90 mL (30 mL×3). The combined organic layer was washed with brine 30 mL (30 mL×3), dried over Na2SO4, filtered and concentrated under reduced pressure to give a residue. The residue was purified by prep-TLC to give tert-butyl 1′H-spiro[azetidine-3,3′-furo[3,4-c]pyridine]-1-carboxylate (50 mg, 0.19 mmol, 77% yield) as a yellow oil. 1H NMR (400 MHz, DMSO-d6) δ 8.80 (d, J=0.4 Hz, 1H), 8.55 (d, J=4.8 Hz, 1H), 7.39 (d, J=4.8 Hz, 1H), 5.08 (s, 2H), 4.26-4.09 (m, 4H), 1.42 (s, 9H).

### Step 3: Preparation of 1′H-spiro[azetidine-3,3′-furo[3,4-c]pyridine]

To a solution of tert-butyl 1′H-spiro[azetidine-3,3′-furo[3,4-c]pyridine]-1-carboxylate (50 mg, 0.19 mmol, 1 eq) in DCM (0.6 mL) was added TFA (0.2 mL). The mixture was stirred at 25° C. for 1 h. The mixture was concentrated directly to give 1′H-spiro[azetidine-3,3′-furo[3,4-c]pyridine](30 mg, crude) as a yellow oil.

### Synthesis of tert-butyl (2R,3S)-2-methyl-3-(pyridin-3-yl)azetidine-1-carboxylate and tert-butyl (2S,3R)-2-methyl-3-(pyridin-3-yl)azetidine-1-carboxylate

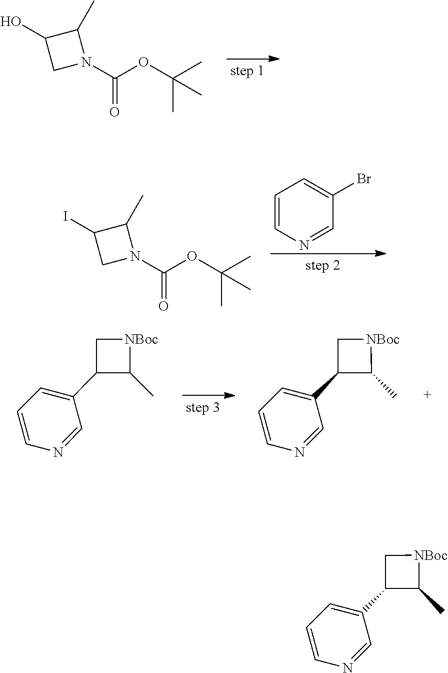

### Step 1: Preparation of Tert-Butyl 3-iodo-2-methylazetidine-1-carboxylate

To a solution of tert-butyl 3-hydroxy-2-methylazetidine-1-carboxylate (5.0 g, 26.7 mmol, 1.0 eq) in DCE (80 mL) was added imidazole (3.6 g, 53.4 mmol, 2.0 eq), PPh3 (10.5 g, 40.0 mmol, 1.5 eq) and iodine (10.2 g, 40.0 mmol, 1.5 eq). The mixture was stirred at 80° C. for 3 h to get black solution. The mixture was added water (100 mL), extracted with EtOAc (150 mL×2), and organic layer was washed with brine (100 mL). The organic layer was combined and dried with anhydrous sodium sulfate. The solid was filtered, solvent was dried under vacuum. The reaction was concentrated in vacuum to get crude product. The residue was purified by silica gel column to give tert-butyl 3-iodo-2-methylazetidine-1-carboxylate (3.5 g, 26.7 mmol, 44% yield) as a colorless oil. 1H NMR (400 MHz, CDCl3) δ 4.56 (t, J=6.0 Hz, 1H), 4.50-4.45 (m, 1H), 4.24-4.14 (m, 1H), 3.99-3.95 (m, 1H), 1.45 (s, 9H), 1.38 (d, J=6.4 Hz, 3H).

### Step 2: Preparation of tert-butyl 2-methyl-3-(pyridin-3-yl)azetidine-1-carboxylate

To a 100 mL vial was added 3-bromopyridine (1.0 g, 6.3 mmol, 1 eq), tert-butyl 3-iodo-2-methylazetidine-1-carboxylate (3.8 g, 13 mmol, 2 eq), Ir[dF(CF3)ppy]2(dtbpy)(PF6) (70 mg, 0.063 mmol, 0.01 eq), NiCl₂·dtbbpy (56 mg, 0.095 mmol, 0.015 eq), TTMSS (1.5 g, 6.3 mmol, 1.0 eq), Na2CO3 (1.3 g, 13 mmol, 2 eq) in DME (60 mL). The vial was sealed under nitrogen. The reaction was stirred and irradiated with a 10 W blue LED lamp (3 cm away), with cooling water to keep the reaction temperature at 25° C. for 14 h. The mixture was concentrated in vacuo. The residue was purified by silica gel column to give tert-butyl 2-methyl-3-(pyridin-3-yl)azetidine-1-carboxylate (550 mg, 2.2 mmol, 35% yield) as a white solid. LCMS (ESI) m/z=249.2 [M+H]+. 1H NMR (400 MHz, CDCl3) δ 8.78 (s, 1H), 8.75 (d, J=5.2 Hz, 1H), 8.28 (d, J=8.0 Hz, 1H), 7.85 (dd, J=5.6, 8.0 Hz, 1H), 4.40-4.25 (m, 2H), 3.92 (dd, J=6.8, 8.8 Hz, 1H), 3.50-3.40 (m, 1H), 1.57 (d, J=6.4 Hz, 3H), 1.48 (s, 9H).

### Step 3: Preparation of Tert-Butyl (2R,3S)-2-methyl-3-(pyridin-3-yl)azetidine-1-carboxylate and tert-butyl (2S,3R)-2-methyl-3-(pyridin-3-yl)azetidine-1-carboxylate

tert-butyl 2-methyl-3-(pyridin-3-yl)azetidine-1-carboxylate (550 mg, 2.2 mmol, 1.0 eq) was separated by SFC (Column: Chiralpak IC-3 50×4.6 mm I.D., 3 um, Mobile phase: Phase A for CO2, and Phase B for MeOH (0.05% DEA); Gradient elution: B in A from 5% to 40%, Flow rate: 3 mL/min; Detector: PDA; Column Temp: 35 C; Back Pressure: 100 Bar).

Peak 1 (RT=1.178 min): tert-butyl (2R,3S)-2-methyl-3-(pyridin-3-yl)azetidine-1-carboxylate or tert-butyl (2S,3R)-2-methyl-3-(pyridin-3-yl)azetidine-1-carboxylate (170 mg, 0.68 mmol, 31% yield) as a colorless oil. LCMS (ESI) m/z=249.2 [M+H]⁺. ¹H NMR (400 MHz, DMSO-d₆) δ 8.64 (s, 1H), 8.58 (d, J=4.4 Hz, 1H), 8.03 (d, J=8.0 Hz, 1H), 7.58 (dd, J=5.2, 8.0 Hz, 1H), 4.21 (t, J=6.2 Hz, 1H), 4.12 (s, 1H), 3.84 (d, J=7.6 Hz, 1H), 3.51-3.41 (m, 1H), 1.44 (d, J=6.4 Hz, 3H), 1.41 (s, 9H).

Peak 2 (RT=1.407 min) as a colorless oil: tert-butyl (2S,3R)-2-methyl-3-(pyridin-3-yl)azetidine-1-carboxylate or tert-butyl (2R,3S)-2-methyl-3-(pyridin-3-yl)azetidine-1-carboxylate (120 mg, 0.48 mmol, 22% yield) as a colorless oil. LCMS (ESI) m/z=249.2 [M+H]⁺. ¹H NMR (400 MHz, DMSO-d₆) δ 8.64 (s, 1H), 8.58 (d, J=4.4 Hz, 1H), 8.03 (d, J=8.0 Hz, 1H), 7.58 (dd, J=5.2, 8.0 Hz, 1H), 4.21 (t, J=6.0 Hz, 1H), 4.12 (s, 1H), 3.84 (d, J=7.6 Hz, 1H), 3.51-3.41 (m, 1H), 1.44 (d, J=6.4 Hz, 3H), 1.41 (s, 9H).

### Synthesis of 5-(azetidin-3-yl)-2-(difluoromethyl)-4-methoxypyridine

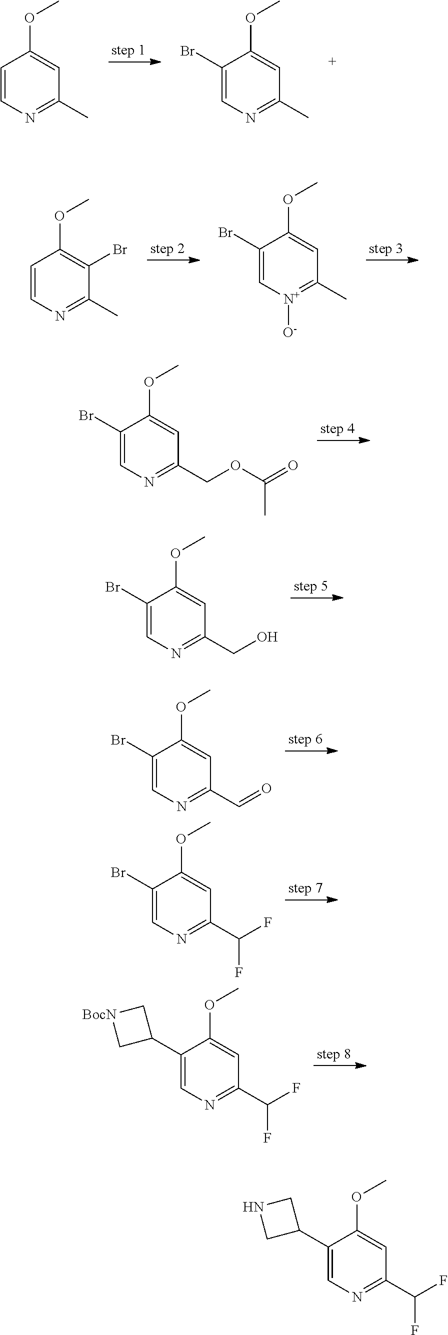

### Step 1: Preparation of 5-bromo-4-methoxy-2-methylpyridine

To a cooled (0° C.) solution of 4-methoxy-2-methylpyridine (10 g, 81 mmol, 1 eq) in AcOH (100 mL) was added bromine (32 g, 0.20 mmol, 2.5 eq). The mixture was subsequently heated to 70° C. for 3 h. The mixture was added cold water (300 mL), the reaction mixture was diluted with water (200 mL) and extracted with EtOAc (500 mL×3). The combined organic layers were dried over anhydrous Na2SO4, filtered and concentrated in vacuo. The residue was purified by column chromatography to give 5-bromo-4-methoxy-2-methylpyridine (4 g, 20 mmol, 25% yield) as a yellow solid and 3-bromo-4-methoxy-2-methylpyridine (2 g, 9.9 mmol, 12% yield) as a yellow solid. 5-bromo-4-methoxy-2-methylpyridine: ¹H NMR (400 MHz, CDCl₃) δ 8.44 (s, 1H), 6.69 (s, 1H), 3.94 (s, 3H), 2.52 (s, 3H). 3-bromo-4-methoxy-2-methylpyridine: ¹H NMR (400 MHz, CDCl₃) δ 8.29 (d, J=5.6 Hz, 1H), 6.69 (d, J=5.6 Hz, 1H), 3.94 (s, 3H), 2.66 (s, 3H)

### Step 2: Preparation of 5-bromo-4-methoxy-2-methylpyridine 1-oxide

To a cooled (0° C.) solution of 5-bromo-4-methoxy-2-methylpyridine (4.0 g, 20 mmol, 1.0 eq) in DCM (40 mL) was added mCPBA (5.1 g, 30 mmol, 1.5 eq) and the mixture was subsequently stirred at 20° C. After 3 h, the reaction mixture was quenched by addition sat. sodium sulfite (100 mL), diluted with water (100 mL), and extracted with DCM/MeOH=10/1 (200 mL×3). The combined organic layers were washed with sat. brine (100 mL×3), dried over anhydrous Na2SO4, filtered and concentrated under reduced pressure to give compound 5-bromo-4-methoxy-2-methylpyridine 1-oxide (4.0 g, crude) as a white solid. 1H NMR (400 MHz, CD3OD) δ 8.48 (s, 1H), 7.26 (s, 1H), 4.00 (s, 3H), 2.50 (s, 3H).

### Step 3: Preparation of (5-bromo-4-methoxypyridin-2-yl)methyl acetate

A mixture of 5-bromo-4-methoxy-2-methylpyridine 1-oxide (2 g, 9.17 mmol, 1 eq), acetyl acetate (5.4 g, 53 mmol, 2.0 eq) in AcOH (15 mL) was stirred at 120° C. for 3 h. The mixture was concentrated under reduced pressure to give compound (5-bromo-4-methoxypyridin-2-yl)methyl acetate (1.50 g, crude) as a yellow oil. LCMS (ESI) m/z=261.8 [M+H]+.

### Step 4: Preparation of (5-bromo-4-methoxypyridin-2-yl)methanol

A mixture of (5-bromo-4-methoxypyridin-2-yl)methyl acetate (2 g, 7.68 mmol, 1 eq), NaOH (10 mL, 2N in water) in MeOH (20 mL) was stirred for 1 h. The mixture was concentrated under reduced pressure to remove MeOH, diluted with water (30 mL), and extracted with EtOAc/MeOH (50 mL×4). The combined organic layer was dried over anhydrous Na2SO4, filtered and concentrated under reduced pressure. The residue was purified by column chromatography to give compound (5-bromo-4-methoxypyridin-2-yl)methanol (1 g, 4.58 mmol, 59.8% yield) as a white solid. 1H NMR (400 MHz, CDCl3) δ 8.47 (s, 1H), 6.85 (s, 1H), 4.69 (s, 2H), 3.95 (s, 3H).

### Step 5: Preparation of 5-bromo-4-methoxypicolinaldehyde

A mixture of (5-bromo-4-methoxypyridin-2-yl)methanol (0.5 g, 2.3 mmol, 1.0 eq), manganese(IV) oxide (4.2 g, 46 mmol, 20 eq) in DCM (5 mL) was stirred for 1 h. The mixture was concentrated under reduced pressure. The residue was purified by column chromatography to give compound 5-bromo-4-methoxypicolinaldehyde (0.25 g, 1.2 mmol, 51% yield) as a white solid. 1H NMR (400 MHz, CDCl3) δ 9.93 (s, 1H), 8.74 (s, 1H), 7.47 (s, 1H), 4.03 (s, 3H).

### Step 6: Preparation of 5-bromo-2-(difluoromethyl)-4-methoxypyridine

To a cooled (0° C.) solution of 5-bromo-4-methoxypicolinaldehyde (0.22 g, 1.0 mmol, 1.0 eq) in DCM (0.5 mL) was added a solution of DAST (0.33 g, 2.0 mmol, 2.0 eq) in DCM (5 mL), After 0.5 h, the reaction mixture was poured into sat. NaHCO₃(5 mL), diluted with water (5 mL) and extracted with DCM (5 mL×2). The combined organic layer was dried over anhydrous Na2SO4, filtered and concentrated under reduced pressure. The residue was purified by column chromatography to give compound 5-bromo-2-(difluoromethyl)-4-methoxypyridine (150 mg, 0.63 mmol, 63% yield) as a colorless oil. 1H NMR (400 MHz, CDCl3) δ 8.60 (s, 1H), 7.14 (s, 1H), 6.71-6.43 (m, 1H), 4.02 (s, 3H).

### Step 7: Preparation of tert-butyl 3-(6-(difluoromethyl)-4-methoxypyridin-3-yl)azetidine-1-carboxylate

To an 15 mL vial equipped with a stir bar was added, 5-bromo-2-(difluoromethyl)-4-methoxypyridine (40 mg, 0.17 mmol, 1 eq), tert-butyl 3-bromoazetidine-1-carboxylate (51 mg, 0.22 mmol, 1.3 eq), Ir[dF(CF3)ppy]2(dtbpy)(PF6) (1.9 mg, 1.7 μmol, 0.01 eq), Na2CO3 (36 mg, 0.34 mmol, 2 eq), NiCl₂·dtbbpy (1 mg, 2.5 μmol, 0.02 eq), 1,1,1,3,3,3-hexamethyl-2-(trimethylsilyl)trisilane (42 mg, 0.17 mmol, 1.0 eq) in DME (2 mL). The vial was sealed and placed under nitrogen was added. The reaction was stirred and irradiated with a 10 W blue LED lamp (3 cm away), with cooling water to keep the reaction temperature at 25° C. for 14 h. The mixture was concentrated under reduced pressure. The residue was purified by prep-TLC to give tert-butyl 3-(6-(difluoromethyl)-4-methoxypyridin-3-yl)azetidine-1-carboxylate (30 mg, 95 μmol, 57% yield) as a yellow oil. 1H NMR (400 MHz, CDCl3) δ 8.36 (s, 1H), 7.12 (s, 1H), 6.73-6.45 (m, 1H), 4.32-4.26 (m, 2H), 4.02-4.00 (m, 1H), 3.95-3.90 (m, 4H), 3.61-3.57 (m, 1H), 1.46 (s, 9H).

### Step 8: Preparation of 5-(azetidin-3-yl)-2-(difluoromethyl)-4-methoxypyridine

A mixture of tert-butyl 3-(6-(difluoromethyl)-4-methoxypyridin-3-yl)azetidine-1-carboxylate (30 mg, 95 μmol, 1 eq) in DCM (0.6 mL) and TFA (0.2 mL) was stirred at 20° C. for 0.5 h. The mixture was concentrated under reduced pressure to give 5-(azetidin-3-yl)-2-(difluoromethyl)-4-methoxypyridine (25 mg, crude) as yellow oil. LCMS (ESI) m/z=215.1 [M+H]+.

### Synthesis of tert-butyl 3-(4-fluoropyridin-3-yl)azetidine-1-carboxylate

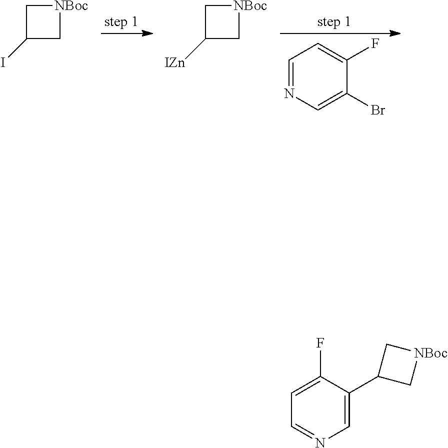

### Step 1: Preparation of (1-(tert-butoxycarbonyl)azetidin-3-yl)zinc(II) iodide

A suspension of Zn (50 g, 764 mmol, 1.3 eq) in aqueous 1M HCl (40 mL) was stirred at 25° C. for 12 h. The suspension was filtered and the filtrate cake was sequentially washed with H2O (40 mL×3), EtOH (40 mL×3), and acetone (40 mL×3). The solid was dried to give a freshly pre-activated Zinc.

A heated (65° C.) suspension Zn (40 g, 611 mmol, 5 eq) in DMF (50 mL) under N₂ was stirred at for 0.5 h, followed by dropwise addition of TMSCl (22 g, 196 mmol, 0.32 eq) and 1,2-dibromoethane (17 g, 92 mmol, 0.15 eq). The resulting mixture was stirred at 60° C. for 0.5 h. A solution of tert-butyl 3-iodoazetidine-1-carboxylate (173 g, 612 mmol, 1 eq) in DMF (300 mL) was added to the mixture and stirred at 60° C. for 0.5 h. The resulting clear yellow solution was cooled to room temperature, filtered and used for the next step without further manipulation.

### Step 2: Preparation of tert-butyl 3-(4-fluoropyridin-3-yl)azetidine-1-carboxylate

To a cooled (0° C.) solution of 3-bromo-4-fluoropyridine (20 g, 114 mmol, 1 eq) and Pd(PPh3)Cl2 (8.0 g, 11 mmol, 0.1 eq) in DMF (200 mL) was added a solution of (1-(tert-butoxycarbonyl)azetidin-3-yl)zinc(II) iodide (350 mL, 570 mmol, 5 eq) in a dropwise manner. The mixture was warmed and stirred at 50° C. for 12 h. The reaction mixture was quenched with water (2 L) and extracted with EtOAc (2 L×3). The combined organic layers were washed with saturated brine (1 L×2), dried over Na2SO4, filtered, and concentrated under reduced pressure. The residue was purified by flash silica gel chromatography and was collected and concentrated in vacuo to give tert-butyl 3-(4-fluoropyridin-3-yl)azetidine-1-carboxylate (20 g, 79 mmol, 70% yield) as yellow oil. LCMS (ESI) m/z=252.9 [M+H]+. 1H NMR (400 MHz, CDCl3) δ 8.60 (d, J=10.0 Hz, 1H), 8.52 (dd, J=5.6, 8.0 Hz, 1H), 7.04 (dd, J=5.6, 10.0 Hz, 1H), 4.41-4.33 (m, 2H), 4.17-4.08 (m, 3H), 1.47 (s, 9H).

The following intermediates in Table 88 were prepared using the method described above for the synthesis of tert-butyl 3-(4-fluoropyridin-3-yl)azetidine-1-carboxylate utilizing the appropriate starting materials and modifications.

TABLE 88

| Name | Structure | LCMS | NMR data |
|---|---|---|---|
| tert-butyl 3-(4- fluoropyridin-2- yl)azetidine-1- carboxylate | 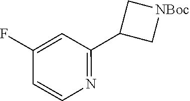 [SMILES: CC(C)(C)OC(=O)N1CC(c2cc(F)ccn2)C1] | 197 [M + H]⁺ | ¹H NMR (400 MHz, CDCl₃) δ 8.54 (s, 1 H) 8.38-8.40 (m, 1 H) 7.08-7.11 (m, 1 H) 4.32-4.37 (m, 2 H) 4.09 (dd, J = 8.0, 6.8 Hz, 2 H) 3.56-3.63 (m, 1 H), 1.46 (s, 9 H) |
| tert-butyl 3-(3- fluoropyridin-2- yl)azetidine-1- carboxylate | 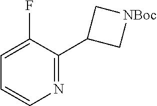 [SMILES: CC(C)(C)OC(=O)N1CC(c2ncccc2F)C1] | 252.2 [M + H]⁺ | ¹H NMR (400 MHz, CD₃OD) δ 8.46-8.38 (m, 1H), 7.60-7.50 (m, 1H), 7.36 (d, J = 4.4 Hz, 1H), 4.30-4.19 (m, 4H), 3.31 (s, 1H), 1.46 (s, 9H) |
| tert-butyl 3-(1- methyl-2-oxo- 1,2- dihydropyridin- 3-yl)azetidine- 1-carboxylate | 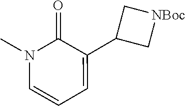 [SMILES: Cn1cccc(C2CN(C(=O)OC(C)(C)C)C2)c1=O] | 209.1 [M + H]⁺ | ¹H NMR (400 MHz, CDCl₃) δ 7.32 (d, J = 7.2 Hz, 1H), 7.22-7.25 (m, 1H), 6.20 (t, J = 6.8 Hz, 1H), 4.64-4.54 (m, 1H), 4.33- 4.25 (m, 2H), 4.20-4.12 (m, 2H), 3.56 (s, 3H), 1.45 (s, 9H). |
| tert-butyl 3-(4- cyanopyrimidin- 2-yl)azetidine- 1-carboxylate | 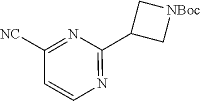 [SMILES: CC(C)(C)OC(=O)N1CC(c2nccc(C#N)n2)C1] | 205.1 [M + H]⁺ | ¹H NMR (400 MHz, CDCl₃) δ 8.97 (d, J = 4.0 Hz, 1H) 7.54 (d, J = 4.0 Hz, 1H) 4.33 (m, 2H) 4.22-4.29 (m, 2H) 4.04-4.14 (m, 1H) 1.47 (s, 9H) |

### Synthesis of 5-(azetidin-3-yl)-2-methoxy-4-methylpyridine

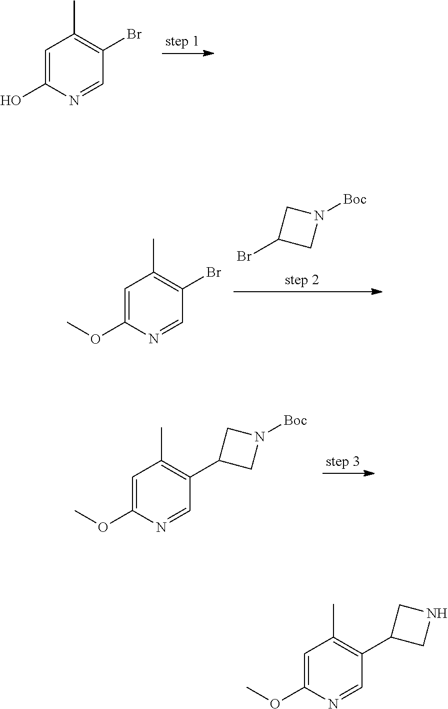

### Step 1: Preparation of 5-bromo-4-methylpyridin-2-yl acetate

To a solution of 5-bromo-4-methylpyridin-2-ol (1.0 g, 5.3 mmol, 1.0 eq) in THF (3.0 mL) was added NaH (0.66 g, 27 mmol, 5.0 eq) at 0° C., the mixture was stirred at 0° C. for 0.5 h, then iodomethane (7.7 g, 53 mmol, 10 eq) was added. The mixture was stirred at 25° C. for 4 h to give a white suspension. The reaction mixture was quenched by water 4 mL at 0° C., then the reaction mixture was concentrated under reduced pressure to give a residue. The residue was purified by reversed phase to lyophilized to give 5-bromo-2-methoxy-4-methylpyridine (0.70 g, 3.46 mmol, 65% yield) as a white solid. 1H NMR (400 MHz, CD3OD) δ 7.90 (s, 1H) 6.50 (s, 1H) 3.52 (s, 3H) 2.27 (s, 3H).

### Step 2: Preparation of tert-butyl 3-(6-methoxy-4-methylpyridin-3-yl)azetidine-1-carboxylate

To an 15 mL vial equipped with a stir bar was added 5-bromo-2-methoxy-4-methylpyridine (0.50 g, 2.5 mmol, 1.0 eq), tert-butyl 3-bromoazetidine-1-carboxylate (0.76 g, 3.2 mmol, 1.3 eq), Ir[dF(CF₃)ppy]₂(dtbpy)(PF₆) (5.8 mg, 24 μmol, 0.010 eq), NiCl₂·dtbbpy (15 mg, 37 μmol, 0.015 eq), TTMSS (0.62 g, 2.5 mmol, 1.0 eq), Na₂CO₃ (0.52 g, 4.9 mmol, 2.0 eq) in DME (25 mL). The vial was sealed and placed under nitrogen was added. The reaction was stirred and irradiated with a 10 W blue LED lamp (3 cm away), with cooling water to keep the reaction temperature at 25° C. for 14 h to give a brown suspension. The reaction mixture was concentrated under reduced pressure to give a residue. The residue was purified by flash silica gel chromatography to tert-butyl 3-(6-methoxy-4-methylpyridin-3-yl)azetidine-1-carboxylate (0.17 g, 0.61 mmol, 25% yield) as a yellow oil. 1H NMR (400 MHz, CD3OD) δ 7.58 (s, 1H) 6.41 (s, 1H) 4.26 (s, 2H) 3.90-4.02 (m, 2H) 3.80-3.89 (m, 1H) 3.56 (s, 3H) 2.12 (s, 3H) 1.45 (s, 9H).

### Step 3: Preparation of 5-(azetidin-3-yl)-4-methylpyridin-2-ol

The tert-butyl 3-(6-methoxy-4-methylpyridin-3-yl)azetidine-1-carboxylate (0.10 g, 0.36 mmol, 1.0 eq) was dissolved in DCM (1 mL) and TFA (0.5 mL) and stirred at 25° C. for 1 h to give a yellow solution. The reaction mixture was concentrated under reduced pressure to give 5-(azetidin-3-yl)-2-methoxy-4-methylpyridine (0.10 g, crude) as a yellow oil. LCMS (ESI) m/z=179.1 [M+H]⁺.

### Synthesis of 1-(3-(azetidin-3-yl)pyridin-4-yl)-3-methylimidazolidin-2-one

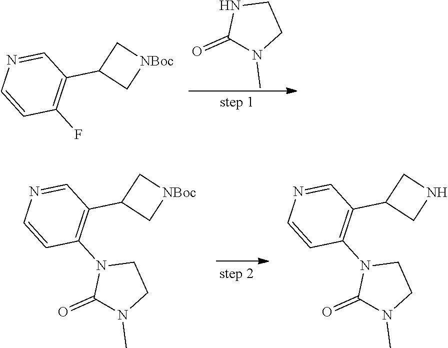

### Step 1: Preparation of Tert-Butyl 3-(4-(3-methyl-2-oxoimidazolidin-1-yl)pyridin-3-yl)azetidine-1-carboxylate

To a solution of 1-methylimidazolidin-2-one (0.36 g, 3.6 mmol, 3.0 eq) in DMF (5.0 mL) at 0° C. was added NaH (0.14 mg, 3.6 mmol, 60% purity, 3.0 eq) under N2 atmosphere, the mixture stirred for 1 h, and then tert-butyl 3-(4-fluoro-3-pyridyl)azetidine-1-carboxylate (0.30 mg, 1.2 mmol, 1.0 eq) was added. The mixture was stirred at 50° C. for 6 h.

The reaction mixture was quenched by addition H2O (10 mL) and extracted with EtOAc (10 mL×3). The combined organic layers were washed with aqueous NaCl (10 mL×3), dried over Na2SO4, filtered, and concentrated under reduced pressure to give a residue. The crude product was purified by reversed-phase HPLC (0.1% FA), then lyophilized to give tert-butyl 3-[4-(3-methyl-2-oxoimidazolidin-1-yl)pyridin-3-yl]azetidine-1-carboxylate (0.18 g, 0.54 mmol, 68% yield) as a yellow solid. LCMS (ESI) m/z=333.3 [M+H]+. 1H NMR (400 MHz, CDCl3) δ 8.52 (s, 1H) 8.35 (d, J=5.6 Hz, 1H) 6.87 (d, J=5.6 Hz, 1H) 4.35-4.38 (m, 2H) 4.04-4.06 (m, 2H) 3.53 (m, 1H) 3.34 (m, 2H) 3.14 (t, J=5.6 Hz, 2H) 2.81 (s, 3H) 1.48 (s, 9H).

The following intermediates in Table 89 were prepared using the method described above in Step 1 for the preparation of tert-butyl 3-(4-(3-methyl-2-oxoimidazolidin-1-yl)pyridin-3-yl)azetidine-1-carboxylate utilizing the appropriate starting materials and modifications.

TABLE 89

| Name | Structure | LCMS | NMR data |
|---|---|---|---|
| tert-butyl 3-(4- (2- oxopyrrolidin- 1-yl)pyridin-3- yl)azetidine-1- carboxylate | 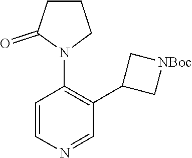 [SMILES: CC(C)(C)OC(=O)N1CC(c2cnccc2N2CCCC2=O)C1] | 318.1 [M + H]⁺ | ¹H NMR (400 MHz, CDCl₃) δ 8.64 (s, 1H), 8.44 (s, 1H), 6.98 (d, J = 4.8 Hz, 1H), 4.24 (t, J = 8.0 Hz, 2H), 3.95-3.89 (m, 2H), 3.88- 3.83 (m, 1H), 3.80-3.65 (m, 2H), 2.54- 2.45 (m, 2H) 2.23-2.13 (m, 2H), 1.36 (s, 9H) |

### Step 2: Preparation of 1-(3-(azetidin-3-yl)pyridin-4-yl)-3-methylimidazolidin-2-one

To a solution of tert-butyl 3-[4-(3-methyl-2-oxoimidazolidin-1-yl)pyridin-3-yl]azetidine-1-carboxylate (0.18 g, 0.54 mmol, 1.0 eq) in methylene chloride (3.0 mL) was added TFA (1.0 mL). The mixture was stirred at 25° C. for 1 h. The reaction mixture was concentrated under reduced pressure to give 1-[3-(azetidin-3-yl)pyridin-4-yl]-3-methylimidazolidin-2-one (0.12 g, crude, TFA salt) as a yellow liquid. LCMS (ESI) m/z=233.2 [M+H]+.

The following intermediates in Table 90 were prepared using the method described above in Step 2 for the preparation of 1-(3-(azetidin-3-yl)pyridin-4-yl)-3-methylimidazolidin-2-one utilizing the appropriate starting materials and modifications.

TABLE 90

| Name | Structure | LCMS |
|---|---|---|
| 1-(3-(azetidin-3-yl) pyridin-4-yl)pyrrolidin- 2-one (TFA salt) | 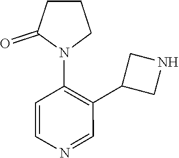 [SMILES: O=C1CCCN1c1ccncc1C1CNC1] | 218.0 [M + H]⁺ |

### Synthesis of (3S,6S,9aS)-6-amino-3-(3-(4-fluoropyridin-3-yl)azetidine-1-carbonyl)hexahydro-1H-pyrrolo[1,2-a]azepin-5(6H)-one

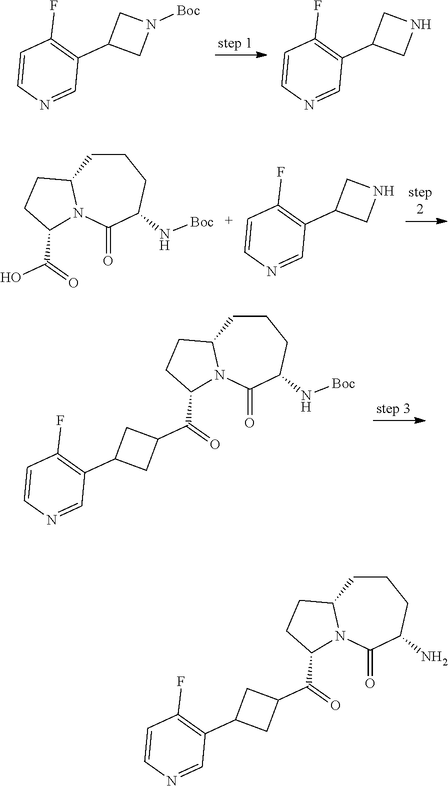

### Step 1: Preparation of 3-(azetidin-3-yl)-4-fluoropyridine

To a stirred solution of tert-butyl 3-(4-fluoropyridin-3-yl)azetidine-1-carboxylate (1.6 g, 6.34 mmol, 1 eq) in CH₂Cl2 (20 mL) was added TFA (3 mL). The resulting yellow solution was stirred for 1 h at room temperature. More TFA (0.5 mL) was added and reaction mixture was stirred for 0.5 h at room temperature. The reaction was concentrated to dryness to give the TFA salt of 3-(azetidin-3-yl)-4-fluoropyridine (964 mg, 100%) that was directly in the next step.

### Step 2: Preparation of Tert-Butyl ((3S,6S,9aS)-3-(3-(4-fluoropyridin-3-yl)azetidine-1-carbonyl)-5-oxooctahydro-1H-pyrrolo[1,2-a]azepin-6-yl)carbamate

To a solution of 3-(azetidin-3-yl)-4-fluoropyridine TFA salt (964 mg, 6.34 mmol, 1.15 eq) in N,N-dimethylformamide (15 mL) were respectively added DIPEA (9 mL, 51.6 mmol, 9.3 eq), (3S,6S,9aS)-6-((tert-butoxycarbonyl)amino)-5-oxooctahydro-1H-pyrrolo[1,2-a]azepine-3-carboxylic acid (1.73 g, 5.53 mmol, 1 eq) and HATU (2.31 g, 6.08 mmol, 1.1 eq). The reaction was stirred at room temperature 16 h. The reaction mixture was concentrated and purified directly by reverse phase chromatography on a 100 g C18 cartridge eluting with 5-60% MeCN in water to afford tert-butyl ((3S,6S,9aS)-3-(3-(4-fluoropyridin-3-yl)azetidine-1-carbonyl)-5-oxooctahydro-1H-pyrrolo[1,2-a]azepin-6-yl)carbamate (1.75 g, 71.1% yield). LCMS (ESI) m/z=447.4 [M+H]+.

### Step 3: Preparation of (3S,6S,9aS)-6-amino-3-(3-(4-fluoropyridin-3-yl)azetidine-1-carbonyl)hexahydro-1H-pyrrolo[1,2-a]azepin-5(6H)-one

To a stirred solution of tert-butyl ((3S,6S,9aS)-3-(3-(4-fluoropyridin-3-yl)azetidine-1-carbonyl)-5-oxooctahydro-1H-pyrrolo[1,2-a]azepin-6-yl)carbamate (1.3 g, 2.91 mmol, 1 eq) in CH₂Cl₂ (20 mL) was added TFA (5 mL). The resulting yellow solution was stirred for 1.5 h at room temperature. The reaction was concentrated to dryness to give the TFA salt of (3S,6S,9aS)-6-amino-3-(3-(4-fluoropyridin-3-yl)azetidine-1-carbonyl)hexahydro-1H-pyrrolo[1,2-a]azepin-5(6H)-one (1.00 g, 100% yield) that was used directly in the next step.

### Synthesis of (3S,6S,9aS)-6-amino-3-(3-(4-(3-methoxyazetidin-1-yl)pyridin-3-yl)azetidine-1-carbonyl)octahydro-5H-pyrrolo[1,2-a]azepin-5-one

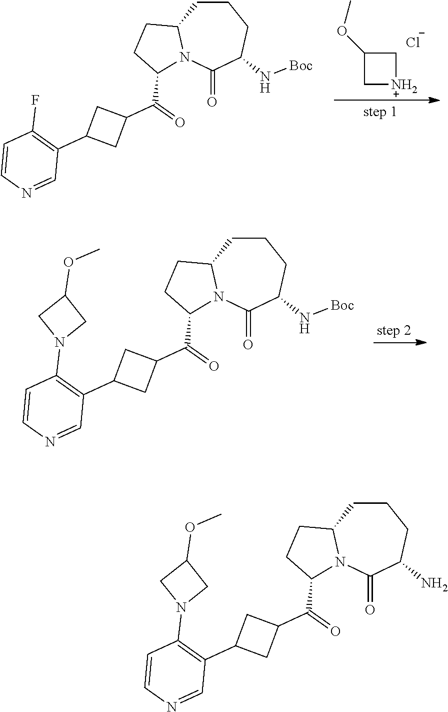

### Step 1: Preparation of Tert-Butyl ((3S,6S,9aS)-3-(3-(4-(3-methoxyazetidin-1-yl)pyridin-3-yl)azetidine-1-carbonyl)-5-oxooctahydro-1H-pyrrolo[1,2-a]azepin-6-yl)carbamate

To a solution of tert-butyl N-[(3S,6S,9aS)-3-[3-(4-fluoropyridin-3-yl)azetidine-1-carbonyl]-5-oxo-octahydro-1H-pyrrolo[1,2-a]azepin-6-yl]carbamate (20 mg, 44.7 μmol, 1 eq) and 3-methoxyazetidine hydrochloride (11.0 mg, 89.4 μmol, 2 eq) in dimethyl sulfoxide (2 mL) was added DIPEA (100 μL, 573 μmol, 13 eq). The resulting solution was heated at 95° C. under nitrogen during 20 h. The crude mixture was purified by reverse phase chromatography on a 50 g C18 cartridge eluting with 5-60% MeCN in water. The pure fractions were concentrated under reduced pressure to give tert-butyl ((3S,6S,9aS)-3-(3-(4-(3-methoxyazetidin-1-yl)pyridin-3-yl)azetidine-1-carbonyl)-5-oxooctahydro-1H-pyrrolo[1,2-a]azepin-6-yl)carbamate (19 mg, 82%) was obtained as a yellow solid. LCMS (ESI) m/z=514.4 [M+H]+.

The following intermediates in Table 91 were prepared using the method described above in Step 1 for the preparation of tert-butyl ((3S,6S,9aS)-3-(3-(4-(3-methoxyazetidin-1-yl)pyridin-3-yl)azetidine-1-carbonyl)-5-oxooctahydro-1H-pyrrolo[1,2-a]azepin-6-yl)carbamate utilizing the appropriate starting materials and modifications.

TABLE 91

| Name | Structure | LCMS |
|---|---|---|
| tert-butyl ((3S,6S,9aS)-3-(3-(4-((S)-2- (methoxymethyl)azetidin-1-yl)pyridin-3- yl)azetidine-1-carbonyl)-5-oxooctahydro-1H- pyrrolo[1,2-a]azepin-6-yl)carbamate | 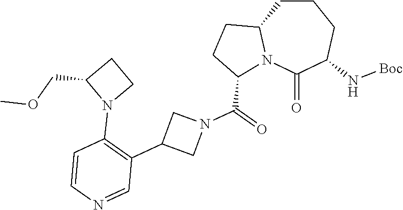 [SMILES: COC[C@@H]1CCN1c1ccncc1C1CN(C(=O)[C@@H]2CC[C@@H]3CCC[C@H](NC(=O)OC(C)(C)C)C(=O)N32)C1] | 528.4 [M + H]⁺ |

### Step 2: Preparation of (3S,6S,9aS)-6-amino-3-(3-(4-(3-methoxyazetidin-1-yl)pyridin-3-yl)azetidine-1-carbonyl)octahydro-5H-pyrrolo[1,2-a]azepin-5-one

To a stirred solution of tert-butyl ((3S,6S,9aS)-3-(3-(4-(3-methoxyazetidin-1-yl)pyridin-3-yl)azetidine-1-carbonyl)-5-oxooctahydro-1H-pyrrolo[1,2-a]azepin-6-yl)carbamate (50 mg, 97.3 μmol, 1 eq) in CH2Cl2 (2.5 mL) was added TFA (800 μL). The resulting yellow solution was stirred for 1 h at room temperature. The reaction was concentrated to dryness to give the TFA salt of (3S,6S,9aS)-6-amino-3-(3-(4-(3-methoxyazetidin-1-yl)pyridin-3-yl)azetidine-1-carbonyl)octahydro-5H-pyrrolo[1,2-a]azepin-5-one. LCMS (ESI) m/z=207.8 (M+2H)2+.

The following intermediates in Table 92 were prepared using the method described above in Step 2 for the preparation of (3S,6S,9aS)-6-amino-3-(3-(4-(3-methoxyazetidin-1-yl)pyridin-3-yl)azetidine-1-carbonyl)octahydro-5H-pyrrolo[1,2-a]azepin-5-one utilizing the appropriate starting materials and modifications.

TABLE 92

| Name | Structure | LCMS |
|---|---|---|
| (3S,6S,9aS)-6-amino-3-(3-(4-((S)-2- (methoxymethyl)azetidin-1-yl)pyridin-3- yl)azetidine-1-carbonyl)octahydro-5H- pyrrolo[1,2-a]azepin-5-one (TFA salt) | 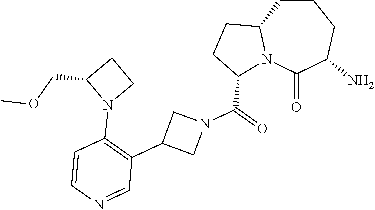 [SMILES: COC[C@@H]1CCN1c1ccncc1C1CN(C(=O)[C@@H]2CC[C@@H]3CCC[C@H](N)C(=O)N32)C1] | 218.4 (M + 2H)²⁺ |

### Synthesis of tert-butyl ((3′S,6′S,10a′S)-3′-(3-methoxy-3-(1-methyl-1H-imidazol-2-yl)azetidine-1-carbonyl)-5′-oxooctahydro-5′H-spiro[cyclopropane-1,8′-pyrrolo[1,2-a]azocin]-6′-yl)carbamate

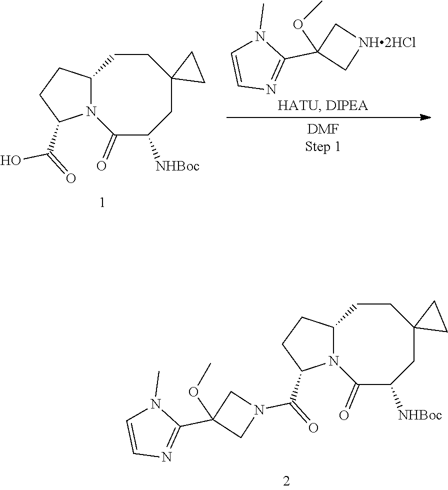

### Step 1: tert-butyl ((3′S,6′S,10a′S)-3′-(3-methoxy-3-(1-methyl-1H-imidazol-2-yl)azetidine-1-carbonyl)-5′-oxooctahydro-5′H-spiro[cyclopropane-1,8′-pyrrolo[1,2-a]azocin]-6′-yl)carbamate

To a solution of (3′S,6′S,10a′S)-6′-((tert-butoxycarbonyl)amino)-5′-oxooctahydro-5′H-spiro[cyclopropane-1,8′-pyrrolo[1,2-a]azocine]-3′-carboxylic acid (20 mg, 56.7 μmol, 1 eq), 2-(3-methoxyazetidin-3-yl)-1-methyl-1H-imidazole dihydrochloride (14.2 mg, 59.5 μmol, 1.05 eq) and HATU (25.8 mg, 68.0 μmol, 1.2 eq) in DMF (3 mL) was added DIPEA (68.8 μL, 396 μmol, 7 eq). The reaction was stirred at room temperature 1 h. The product was purified directly by reverse phase chromatography on a 50 g C18 cartridge eluting with a gradient of 5-60% acetonitrile in water to afford after freeze drying tert-butyl ((3′S,6′S,10a′S)-3′-(3-methoxy-3-(1-methyl-1H-imidazol-2-yl)azetidine-1-carbonyl)-5′-oxooctahydro-5′H-spiro[cyclopropane-1,8′-pyrrolo[1,2-a]azocin]-6′-yl)carbamate (26.0 mg, 91.5%) as a white solid. LCMS (ESI) m/z=502.4 [M+H]⁺.

### Synthesis of racemic-(3S,4R)-4-(1-methyl-2-oxo-1,2-dihydropyridin-3-yl)pyrrolidine-3-carbonitrile

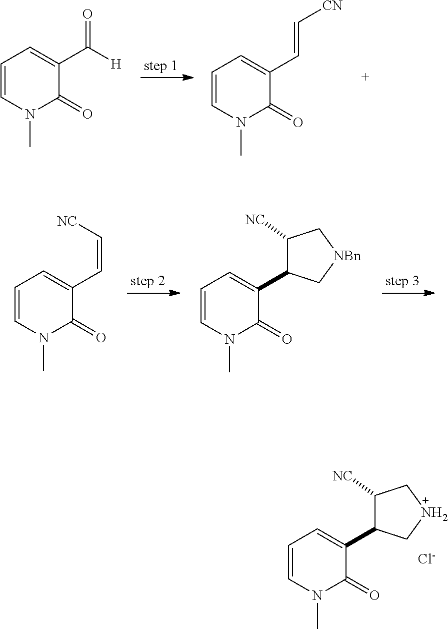

### Step 1: Preparation of Cis- and Trans-3-(2-methoxypyridin-3-yl)prop-2-enenitrile

To a solution of diethyl (cyanomethyl)phosphonate (676 mg, 3.82 mmol, 1.05 eq) in THF (20 mL) was added KOtBu (448 mg, 4.0 mmol, 1.1 eq) at 0° C. The solution was stirred for 10 min at 0° C., followed by addition of 2-methoxypyridine-3-carbaldehyde (500 mg, 3.64 mmol, 1 eq). The reaction was stirred 1 h. Water (100 mL) was added and the product was extracted with EtOAc (2×70 mL). The organic layers were dried with sodium sulfate, filtered and concentrated under reduced pressure to afford a mixture of cis- and trans-3-(2-methoxypyridin-3-yl)prop-2-enenitrile (560 mg, 96.0% yield) as an oil. LCMS (ESI) m/z=161.2 [M+H]+.

### Step 2: Preparation of Racemic-(3S,4R)-1-benzyl-4-(1-methyl-2-oxo-1,2-dihydropyridin-3-yl)pyrrolidine-3-carbonitrile

To a solution of cis- and trans-3-(1-methyl-2-oxo-1,2-dihydropyridin-3-yl)prop-2-enenitrile (0.56 g, 3.49 mmol, 1 eq) and benzyl(methoxymethyl)[(trimethylsilyl)methyl]amine (1.33 mL, 5.23 mmol, 1.5 eq) in DCM (15 mL) was added TFA (4 mL, 52.2 mmol, 10 eq) dropwise at room temperature. After the addition, the solution was stirred 15 min at room temperature. The reaction was quenched with saturated aqueous solution of NaHCO₃ with vigorous stirring and the product was extracted with DCM (2×50 mL). The combined organic layers were concentrated to dryness and the product was purified twice by reverse phase chromatography on a 150 g Cis cartridge eluting with 5-70% acetonitrile in water (10 mM NH₄HCO₃ (pH=10) buffer) to afford racemic-(3S,4R)-1-benzyl-4-(1-methyl-2-oxo-1,2-dihydropyridin-3-yl)pyrrolidine-3-carbonitrile (238 mg, 23.3%) as an off-white solid. 1H NMR (400 MHz, acetone-d6) δ 7.57 (dd, J=6.8, 2.0 Hz, 1H), 7.46 (dd, J=7.0, 1.8 Hz, 1H), 7.40-7.35 (m, 2H), 7.35-7.29 (m, 2H), 7.28-7.20 (m, 1H), 6.18 (t, J=6.8 Hz, 1H), 3.70 (s, 2H), 3.66-3.57 (m, 1H), 3.50 (s, 3H), 3.49-3.44 (m, 1H), 3.09-3.03 (m, 1H), 3.02-2.96 (m, 2H), 2.70-2.62 (m, 1H). LCMS (ESI) m/z=294.2 [M+H]+.

racemic-(3S,4S)-1-Benzyl-4-(1-methyl-2-oxo-1,2-dihydropyridin-3-yl)pyrrolidine-3-carbonitrile (88 mg, 8.6%) was also isolated as an oil. LCMS (ESI) m/z=294.2 [M+H]+.

### Step 3: Preparation of (3S,4R)-4-(1-methyl-2-oxo-1,2-dihydropyridin-3-yl)pyrrolidine-3-carbonitrile

To a solution of (3S,4R)-1-benzyl-4-(1-methyl-2-oxo-1,2-dihydropyridin-3-yl)pyrrolidine-3-carbonitrile (75 mg, 0.2556 mmol, 1 eq) in 1,1-dichloroethane (3 mL) at 0° C. was slowly added 1-chloroethyl chloroformate (35.7 μL, 0.3322 mmol, 1.3 eq). The reaction mixture was stirred for 20 min. at 0° C. and subsequently warmed to room temperature and stirred for 30 min. Additional 1-chloroethyl chloroformate (35.7 μL, 0.3322 mmol, 1.3 eq) was added and the reaction was stirred for 2 h at room temperature. The reaction was heated to reflux for 30 min. and then cooled to room temperature and stirred for 20 h. The reaction was concentrated under reduced pressure. The crude residue was dissolved in methanol (3 mL) and heated to reflux for 1.5 h. The reaction was concentrated under reduced pressure to give crude (3S,4R)-4-(1-methyl-2-oxo-1,2-dihydropyridin-3-yl)pyrrolidine-3-carbonitrile (61 mg, 99%, HCl) as a thick oil. LCMS (ESI) m/z=204.2 [M+H]+.

### Synthesis of tert-butyl N-[(3S,6S,10aS)-3-[(rel-trans)-3-cyano-4-(2-oxo-1,2-dihydropyridin-4-yl)pyrrolidine-1-carbonyl]-5-oxo-decahydropyrrolo[1,2-a]azocin-6-yl]carbamate

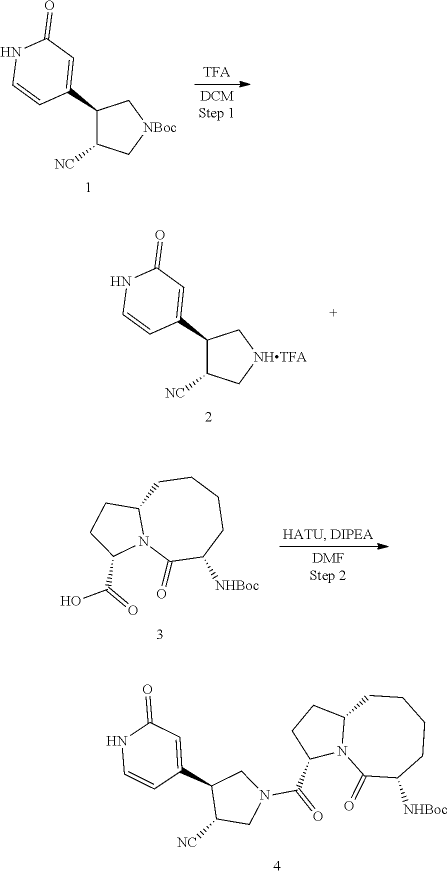

### Step 1: Preparation of (rel-trans)-4-(2-oxo-1,2-dihydropyridin-4-yl)pyrrolidine-3-carbonitrile

To a solution of tert-butyl (rel-trans)-3-cyano-4-(2-oxo-1,2-dihydropyridin-3-yl)pyrrolidine-1-carboxylate (635 mg, 2.19 mmol, 1 eq) in DCM (7 mL) was added TFA (3.34 mL, 43.8 mmol, 20 eq). The solution was stirred at room temperature for 2 h. The reaction was concentrated to dryness to give the crude TFA salt of (rel-trans)-4-(2-oxo-1,2-dihydropyridin-4-yl)pyrrolidine-3-carbonitrile (665 mg, 100%). LCMS (ESI): m/z=190.2 [M+H]+.

### Step 2: Preparation of tert-butyl N-[(3S,6S,10aS)-3-[(3R,4S)-3-cyano-4-(2-oxo-1,2-dihydropyridin-4-yl)pyrrolidine-1-carbonyl]-5-oxo-decahydropyrrolo[1,2-a]azocin-6-yl]carbamate

To a solution of (rel-trans)-4-(2-oxo-1,2-dihydropyridin-4-yl)pyrrolidine-3-carbonitrile TFA salt (665 mg, 3.36 mmol, 1.1 eq) in DMF (30 mL) were added (3S,6S,10aS)-6-((tert-butoxycarbonyl)amino)-5-oxodecahydropyrrolo[1,2-a]azocine-3-carboxylic acid (1 g, 3.06 mmol, 1 eq), DIPEA (3.15 g, 24.4 mmol, 8 eq) and HATU (1.16 g, 3.06 mmol, 1 eq). The reaction was stirred at room temperature for 0.5 h. The crude mixture was concentrated and purified directly by reverse phase chromatography on a 100 g Cis cartridge using a gradient of 5-80% MeCN in water (with 0.1% formic acid) to give tert-butyl N-[(3S,6S,10aS)-3-[(rel-trans)-3-cyano-4-(2-oxo-1,2-dihydropyridin-4-yl)pyrrolidine-1-carbonyl]-5-oxo-decahydropyrrolo[1,2-a]azocin-6-yl]carbamate (500 mg, 32.8%) as a beige solid. LCMS (ESI): m/z=498.4 [M+H]⁺.

### Synthesis of tert-butyl N-[(3S,6S,10aS)-3-[(rel-trans)-3-cyano-4-(1-methyl-2-oxo-1,2-dihydropyridin-4-yl)pyrrolidine-1-carbonyl]-5-oxo-decahydropyrrolo[1,2-a]azocin-6-yl]carbamate

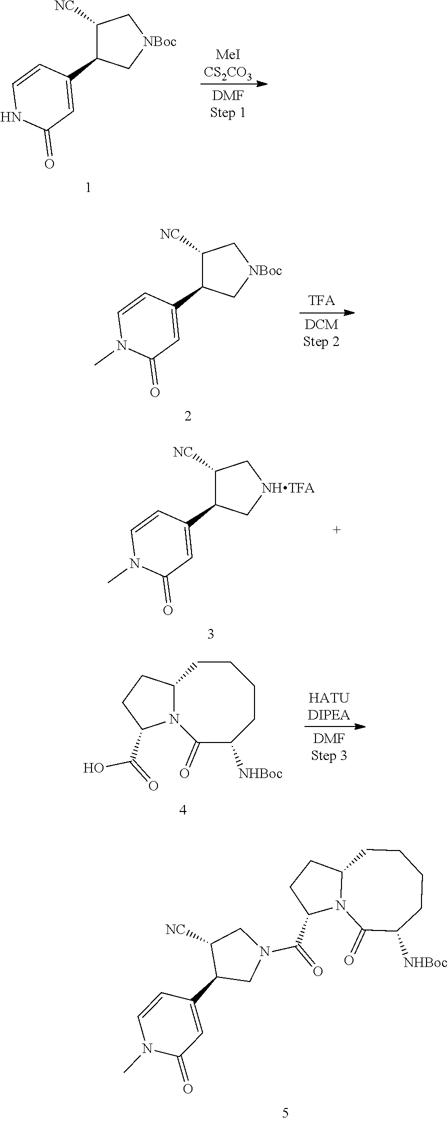

### Step 1: tert-Butyl (rel-trans)-3-cyano-4-(1-methyl-2-oxo-1,2-dihydropyridin-4-yl)pyrrolidine-1-carboxylate

To a solution of tert-butyl (rel-trans)-3-cyano-4-(2-oxo-1,2-dihydropyridin-4-yl)pyrrolidine-1-carboxylate (500 mg, 1.72 mmol, 1 eq) and cesium carbonate (781 mg, 2.40 mmol, 1.4 eq) in N,N-dimethylformamide (8 mL) at 0° C. was added dropwise methyl iodide (159 μL, 2.57 mmol, 1.5 eq). The reaction mixture was stirred at room temperature for 6 h. The crude material was filtered and purified by reverse phase chromatography on a 100 g C18 cartridge eluting with 5-100% MeCN in water (with 0.1% formic acid). The combined fractions were concentrated under reduced pressure to give tert-butyl (rel-trans)-3-cyano-4-(1-methyl-2-oxo-1,2-dihydropyridin-4-yl)pyrrolidine-1-carboxylate (405 mg, 77.7%) as a white solid. LCMS (ESI): m/z=304.3 [M+H]⁺. 1H NMR (400 MHz, DMSO-d6) δ 7.71 (d, J=6.8 Hz, 1H), 6.40 (s, 1H), 6.29 (d, J=6.6 Hz, 1H), 3.83 (br. s., 1H), 3.69 (d, J=8.3 Hz, 2H), 3.57-3.45 (m, 1H), 3.40 (s, 3H), 3.18 (d, J=7.8 Hz, 1H), 2.50 (s., 1H), 1.40 (s, 9H).

### Step 2: (rel-trans)-4-(1-Methyl-2-oxo-1,2-dihydropyridin-4-yl)pyrrolidine-3-carbonitrile

To a solution of tert-butyl (rel-trans)-3-cyano-4-(1-methyl-2-oxo-1,2-dihydropyridin-4-yl)pyrrolidine-1-carboxylate (400 mg, 1.31 mmol, 1 eq) in methylene chloride (3 mL) was added TFA (1 mL, 13.1 mmol, 10 eq). The resulting yellow solution was stirred for 30 min. at room temperature. The reaction mixture was concentrated under reduced pressure. Crude TFA salt (418 mg, 100%) was used directly in the next step. LCMS (ESI): m/z=204.2 [M+H]+.

### Step 3: tert-Butyl N-[(3S,6S,10aS)-3-[(rel-trans)-3-cyano-4-(1-methyl-2-oxo-1,2-dihydropyridin-4-yl)pyrrolidine-1-carbonyl]-5-oxo-decahydropyrrolo[1,2-a]azocin-6-yl]carbamate

HATU (577 mg, 1.52 mmol, 1.2 eq) was added to a solution of (rel-trans)-4-(1-methyl-2-oxo-1,2-dihydropyridin-4-yl)pyrrolidine-3-carbonitrile TFA salt (260 mg, 1.27 mmol, 1 eq), (3S,6S,10aS)-6-((tert-butoxycarbonyl)amino)-5-oxodecahydropyrrolo[1,2-a]azocine-3-carboxylic acid (453 mg, 1.39 mmol, 1.1 eq) and N,N-diisopropylethylamine (1.32 mL, 7.62 mmol, 6 eq) in N,N-dimethylformamide (10 mL). The reaction mixture was stirred at room temperature for 2 h. The crude product was purified directly by reverse phase chromatography with a 100 g C18 cartridge using a gradient of MeCN in water. The combined fractions were concentrated under reduced pressure and freeze-dried to afford tert-butyl N-[(3S,6S,10aS)-3-[(rel-trans)-3-cyano-4-(1-methyl-2-oxo-1,2-dihydropyridin-4-yl)pyrrolidine-1-carbonyl]-5-oxo-decahydropyrrolo[1,2-a]azocin-6-yl]carbamate (595 mg, 91.6%) as a white solid. LCMS (ESI): m/z=512.4 [M+H]+.

### Synthesis of (rel-trans)-4-(2-methoxypyridin-3-yl)pyrrolidine-3-carbonitrile hydrochloride

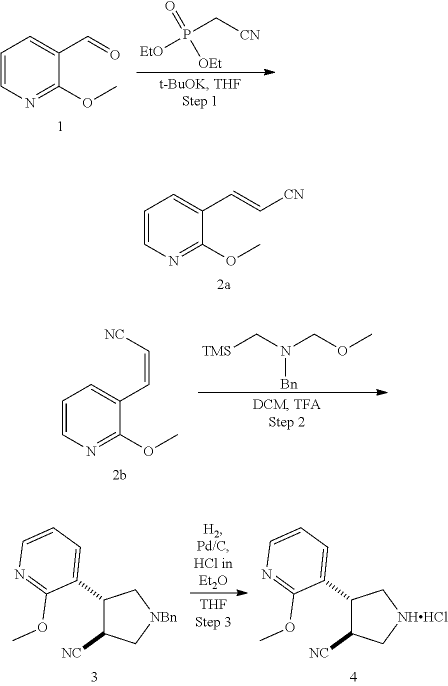

### Step 1: 3-(2-methoxypyridin-3-yl)acrylonitrile

To a solution of diethyl (cyanomethyl)phosphonate (3.38 g, 19.1 mmol, 1.05 eq) in THF (100 mL) was added potassium t-butoxide (2.24 g, 20.0 mmol, 1.1 eq) at 0° C. The solution was stirred for 10 min. at 0° C. and then 2-methoxynicotinaldehyde (2.5 g, 18.2 mmol, 1 eq) was added to the solution. The reaction was stirred 3.5 h. Water (250 mL) was added and the product was extracted with EtOAc (2×150 mL). The organic layers were dried with sodium sulfate, filtered and concentrated under reduced pressure to afford a mixture of cis- and trans-3-(2-methoxypyridin-3-yl)acrylonitrile (3.20 g, 109%) as an oil. LCMS (ESI) m/z=161.2

### Step 2: trans-1-benzyl-4-(2-methoxypyridin-3-yl)pyrrolidine-3-carbonitrile

To a solution of cis- and trans-3-(2-methoxypyridin-3-yl)acrylonitrile (4.84 g, 30.2 mmol, 1 eq) and benzyl(methoxymethyl)[(trimethylsilyl)methyl]amine (9.3 g, 39.2 mmol, 1.3 eq) in DCM (50 mL) was added TFA (4 mL, 52.2 mmol, eq) dropwise at room temperature. After the addition, the solution was stirred 15 min. at room temperature. The reaction was quenched with a saturated aqueous solution of sodium bicarbonate with vigorous stirring and the product was extracted with DCM (3×75 mL). The combined organic layers were concentrated to dryness and the product was purified twice by reverse phase chromatography on a 275 g C18 cartridge eluting with a gradient of 5-70% acetonitrile in basic water (10 mM NH₄HCO₃ (pH=10) buffer) to afford trans-1-benzyl-4-(1-methyl-2-oxo-1,2-dihydropyridin-3-yl)pyrrolidine-3-carbonitrile (2.37 g, 26.7%) as an off-white solid (racemic trans product). 1H NMR (400 MHz, (CD₃)₂CO) δ ppm 8.07 (dd, J=5.0, 1.6 Hz, 1H), 7.71 (dd, J=7.3, 1.2 Hz, 1H), 7.43-7.30 (m, 4H), 7.29-7.23 (m, 1H), 6.94 (dd, J=7.2, 5.0 Hz, 1H), 3.94 (s, 3H), 3.82-3.69 (m, 3H), 3.34-3.26 (m, 1H), 3.08 (dt, J=12.5, 8.7 Hz, 2H), 2.95 (dd, J=9.3, 5.4 Hz, 1H), 2.72 (dd, J=9.2, 7.2 Hz, 1H).

### Step 3: trans-4-(2-methoxypyridin-3-yl)pyrrolidine-3-carbonitrile hydrochloride

To a solution of trans-1-benzyl-4-(1-methyl-2-oxo-1,2-dihydropyridin-3-yl)pyrrolidine-3-carbonitrile (100 mg, 340 μmol, 1 eq) in THF (5 mL) under nitrogen atmosphere were added 2 M HCl in diethyl ether (170 μL, 340 μmol, 1.0 eq) and 10% palladium on carbon (50% wet) (75 mg, 35 μmol, 0.1 eq). The suspension was stirred 16 h under hydrogen atmosphere (1 atm). The reaction mixture was filtered through Celite. The filter cake was washed with THF and the filtrate was concentrated to dryness to provide crude trans-4-(2-methoxypyridin-3-yl)pyrrolidine-3-carbonitrile hydrochloride that was used directly in the next step.

### Synthesis of (rel-trans)-6-phenyl-4-azaspiro[2.4]heptane-7-carbonitrile TFA salt

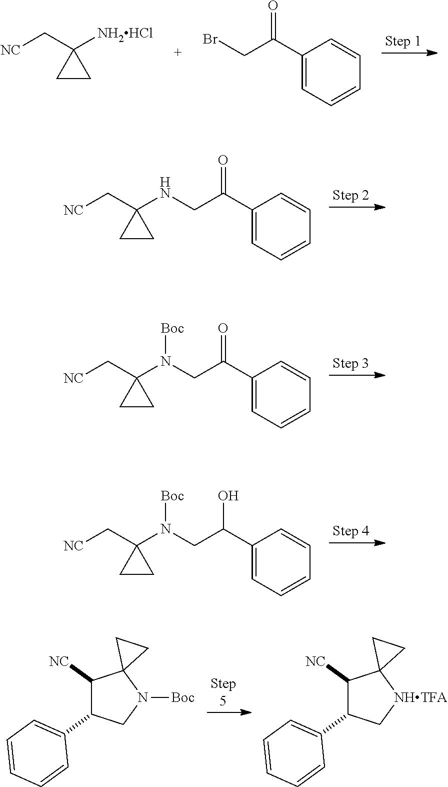

### Step 1: 2-{1-[(2-oxo-2-phenylethyl)amino]cyclopropyl}acetonitrile

To a solution of 2-(1-aminocyclopropyl)acetonitrile hydrochloride (10.7 g, 81.0 mmol, 1 eq) in N,N-dimethylformamide (250 mL) were added 2-bromo-1-phenylethan-1-one (16.1 g, 81.0 mmol, 1 eq) followed by potassium phosphate tribasic (42.8 g, 202 mmol, 2.5 eq). The mixture was stirred at room temperature for 4 h. A yellowish mixture was observed over time. The reaction was quenched with the addition of 1 N HCl (600 mL, pH=1) at room temperature. The aqueous solution was washed with EtOAc (2×500 mL) then basified with 1 N NaOH aqueous solution (until pH=9, 600 mL) then extracted with EtOAc (2×600 mL). The combined organic extracts were washed with brine (2×300 mL), dried over sodium sulfate, filtered and concentrated under reduced pressure to give crude 2-{1-[(2-oxo-2-phenylethyl)amino]cyclopropyl}acetonitrile (12.5 g, 72%) as a clear pale orange liquid. LCMS (ESI) m/z=215.2 [M+H]+. 1H NMR (400 MHz, DMSO-d6) δ 7.97 (d, J=7.6 Hz, 2H), 7.66 (t, J=7.1 Hz, 1H), 7.54 (t, J=7.1 Hz, 2H), 4.25 (s, 2H), 2.79 (s, 2H), 0.76-0.70 (m, 2H), 0.61-0.56 (m, 1H).

### Step 2: tert-butyl N-[1-(cyanomethyl)cyclopropyl]-N-(2-oxo-2-phenylethyl)carbamate

To a solution of 2-{1-[(2-oxo-2-phenylethyl)amino]cyclopropyl}acetonitrile (12.5 g, 58.3 mmol, 1 eq) in THF (150 mL) were added water (150 mL) and sodium bicarbonate (4.89 g, 58.3 mmol, 1.0 eq) followed by di-tert-butyl dicarbonate (25.3 g, 116 mmol, 2 eq). The reaction was stirred for 48 h at room temperature. The reaction was concentrated under reduced pressure (to remove THF) then diluted with EtOAc (250 mL) and water (50 mL). The phases were separated and the aqueous phase was back-extracted with EtOAc (2×250 mL). The combined organic layers were dried with sodium sulfate, filtered and concentrated under reduced pressure. The crude residue was purified flash-chromatography on silica gel eluting with 1-25% EtOAc in heptane to give tert-butyl N-[1-(cyanomethyl)cyclopropyl]-N-(2-oxo-2-phenylethyl)carbamate (14.3 g, 78%) as a white solid. LCMS (ESI) m/z=215.2 (M-Boc+2H), 259.2 (M−t−Bu+2H). ¹H NMR (400 MHz, DMSO-d₆ at 80° C.) δ 7.97 (d, J=7.2 Hz, 2H), 7.67 (t, J=7.1 Hz, 1H), 7.55 (t, J=7.1 Hz, 2H), 4.73 (s, 2H), 2.87 (s, 2H), 1.54-1.24 (m, 9H), 1.04-0.99 (m, 2H), 0.94-0.90 (m, 2H).

### Step 3: tert-butyl N-[1-(cyanomethyl)cyclopropyl]-N-(2-hydroxy-2-phenylethyl)carbamate

To a solution of tert-butyl N-[1-(cyanomethyl)cyclopropyl]-N-(2-oxo-2-phenylethyl)carbamate (14.33 g, 45.4 mmol, 1 eq) in methanol (260 mL) at 0° C. was added sodium borohydride (1.71 g, 45.4 mmol, 1.0 eq) portion wise over 10 min. The reaction was stirred for 20 min at room temperature. The reaction was quenched with 1 N HCl (100 mL) and concentrated under reduced pressure (to remove MeOH). The crude residue was extracted with EtOAc (3×200 mL). The combined organic layers were over sodium sulfate, filtered and concentrated under reduced pressure to give tert-butyl N-[1-(cyanomethyl)cyclopropyl]-N-(2-hydroxy-2-phenylethyl)carbamate (14.7 g, 100%) as a clear pale pink oil. 1H NMR (400 MHz, DMSO-d6 at 80° C.) δ 7.38-7.23 (m, 5H), 5.51 (br. s, 1H), 4.89-4.72 (m, 1H), 3.39-3.27 (m, 1H), 3.21-3.13 (m, 1H), 3.04-2.90 (m, 1H), 2.68-2.60 (m, 1H), 1.48 (s, 9H), 0.83-0.65 (m, 4H).

### Step 4: tert-butyl (rel-trans)-7-cyano-6-phenyl-4-azaspiro[2.4]heptane-4-carboxylate

To a solution of tert-butyl N-[1-(cyanomethyl)cyclopropyl]-N-(2-hydroxy-2-phenylethyl)carbamate (12 g, 37.9 mmol, 1 eq) in THF (650 mL) at −15° C. were added diethyl phosphorochloridate (5.74 mL, 39.7 mmol, 1.05 eq) followed by dropwise addition of 1 M LiHMDS in THF (94.7 mL, 94.7 mmol, 2.5 eq) over 30 min. The reaction was stirred at −15° C. for 30 min. The reaction was quenched with 1N HCl (200 mL) and concentrated under reduced pressure (to remove THF). The crude residue was purified flash-chromatography on silica gel eluting with 1-8% EtOAc in heptane to give rac-tert-butyl (rel-trans)-7-cyano-6-phenyl-4-azaspiro[2.4]heptane-4-carboxylate (3.7 g, 33%) as a clear oil. LCMS (ESI) m/z=243.2 (M−t−Bu+2H)+. ¹H NMR (400 MHz, DMSO-d₆) δ 7.59-7.21 (m, 5H), 4.01 (d, J=10.5 Hz, 1H), 3.90 (dd, J=10.5, 7.8 Hz, 1H), 3.69 (q, J=10.2 Hz, 1H), 3.45 (t, J=10.0 Hz, 1H), 1.96-1.83 (m, 1H), 1.48-1.41 (m, 1H), 1.40-1.32 (m, 9H), 0.92-0.77 (m, 1H), 0.74-0.61 (m, 1H).

rac-tert-butyl (6R,7S)-7-cyano-6-phenyl-4-azaspiro[2.4]heptane-4-carboxylate was submitted to chiral SFC separation (SFC conditions: Column Lux Amylose 1, 30×250 mm 5 um column, 9.25 mg/inj, concentration 18.5 mg/mL, Column T=40° C., Flow rate 100 mL/min, 10% MeOH, cycle time: 2 min) to give tert-butyl (6R,7S)-7-cyano-6-phenyl-4-azaspiro[2.4]heptane-4-carboxylate (1.57 g, 5.26 mmol, 42% recovery, 99.9% ee) as a white solid and tert-butyl (6S,7R)-7-cyano-6-phenyl-4-azaspiro[2.4]heptane-4-carboxylate (1.57 g, 0.70 mmol, 42% recovery, 99.9% ee) as a white solid.

The assignment of absolute stereochemical configuration was made by comparing experimental vibrational circular dichroism (VCD) spectra with theoretical VCD spectra obtained from DFT calculations.

### Step 5: (rel-trans)-6-phenyl-4-azaspiro[2.4]heptane-7-carbonitrile TFA salt

To a solution of a tert-butyl (rel-trans)-7-cyano-6-phenyl-4-azaspiro[2.4]heptane-4-carboxylate (32 mg, 0.1072 mmol, 1 eq) in methylene chloride (4 mL) was added TFA (1 mL). The reaction was stirred at room temperature for 2 h. The reaction was concentrated under reduced pressure to give crude (rel-trans)-6-phenyl-4-azaspiro[2.4]heptane-7-carbonitrile TFA salt (21.2 mg, 99%) as a thick oil. LCMS (ESI) m/z=229.2 [M+H]+.

The compounds in Table 93 were prepared with the procedures described for tert-butyl (rel-trans)-7-cyano-6-phenyl-4-azaspiro[2.4]heptane-4-carboxylate utilizing the appropriate starting materials.

TABLE 93

| Name | Structure | LCMS |
|---|---|---|
| tert-butyl (6S,7R)-7-cyano- 6-(p-tolyl)-4- azaspiro[2.4]heptane-4- carboxylate | 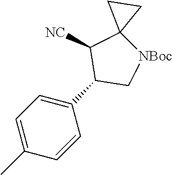 [SMILES: Cc1ccc([C@H]2CN(C(=O)OC(C)(C)C)C3(CC3)[C@@H]2C#N)cc1] | 313 [M + H]⁺ |
| | SFC Peak 1 | |
| | (absolute configuration | |
| | artibrarily assigned) | |
| tert-butyl (6R,7S)-7-cyano- 6-(p-tolyl)-4- azaspiro[2.4]heptane-4- carboxylate | 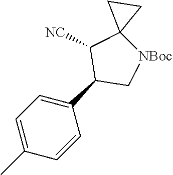 [SMILES: Cc1ccc([C@@H]2CN(C(=O)OC(C)(C)C)C3(CC3)[C@H]2C#N)cc1] | 313 [M + H]⁺ |
| | SFC Peak 2 | |
| | (absolute configuration | |
| | arbitrarily assigned) | |

### Synthesis of 6-(5-fluoro-1-methyl-2-oxo-1,2-dihydropyridin-4-yl)-4,6-diazaspiro[2.4]heptane-5,7-dione

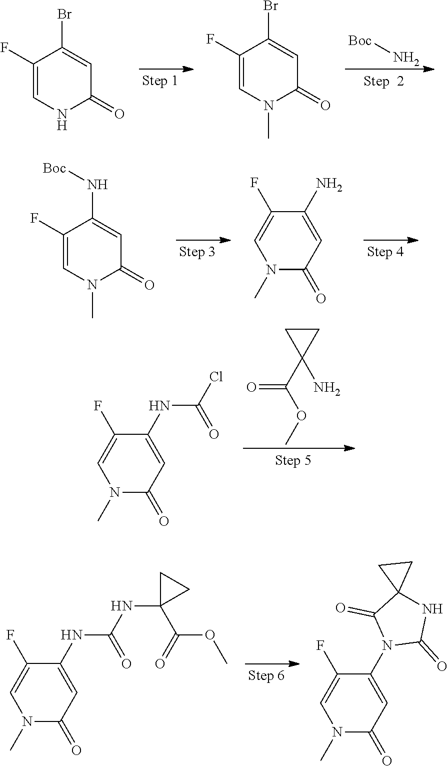

### Step 1: Preparation of 4-bromo-5-fluoro-1-methylpyridin-2(1H)-one

To a solution of 4-bromo-5-fluoropyridin-2(1H)-one (1.5 g, 7.8 mmol, 1 eq) in DMF (20 mL) was added dipotassium carbonate (1.4 g, 10 mmol, 1.3 eq) and iodomethane (1.1 g, 7.8 mmol, 1.0 eq), the mixture was stirred at 25° C. for 16 h. The reaction was diluted with water (100 mL) and extracted with EtOAc (100 mL×2), the combined organic layers were washed with saturated brine (50 mL×2), dried over Na2SO4, filtered and concentrated under reduced pressure to give a residue. The residue was purified by flash column chromatography to give 4-bromo-5-fluoro-1-methylpyridin-2(1H)-one (1.4 g, 6.8 mmol, 87% yield) as a white solid. LCMS (ESI) m/z=206.1 [M+H]+. 1H NMR (400 MHz, CD₃OD) δ 7.87 (d, J=4.4 Hz, 1H), 6.88 (d, J=6.4 Hz, 1H), 3.51 (s, 3H).

### Step 2: Preparation of Tert-Butyl (5-fluoro-1-methyl-2-oxo-1,2-dihydropyridin-4-yl)carbamate

To a solution of 4-bromo-5-fluoro-1-methylpyridin-2(1H)-one (1.4 g, 6.8 mmol, 1 eq) in dioxane (20 mL) was added [5-(diphenylphosphanyl)-9,9-dimethyl-9H-xanthen-4-yl]diphenylphosphane (0.8 g, 1.4 mmol, 0.2 eq), tert-butyl carbamate (1.6 g, 13.5 mmol, 2 eq), Cs₂CO₃ (6.6 g, 20.3 mmol, 3 eq) and Pd₂(dba)₃ (621 mg, 679 μmol, 0.1 eq). The mixture was degassed and purged with N₂ (3 times), and then the mixture was stirred at 100° C. for 12 h. The reaction mixture was filtered and the filter was concentrated to give a residue. The residue was purified by flash column chromatography to give a residue. The residue was purified by reversed phase (TFA) then lyophilization to give tert-butyl (5-fluoro-1-methyl-2-oxo-1,2-dihydropyridin-4-yl)carbamate (1.4 g, 5.8 mmol, 85% yield) as a white solid. LCMS (ESI) m/z=242.9 [M+H]⁺.

### Step 3: Preparation of 4-amino-5-fluoro-1-methylpyridin-2(1H)-one

To a solution of tert-butyl (5-fluoro-1-methyl-2-oxo-1,2-dihydropyridin-4-yl)carbamate (1.4 g, 5.8 mmol, 1 eq) in DCM (12 mL) was added TFA (3 mL), the mixture was stirred at 25° C. for 1 h to give a clear brown solution. The reaction mixture was concentrated under reduced pressure to give 4-amino-5-fluoro-1-methylpyridin-2(1H)-one (1.4 g, crude) as brownness oil. LCMS (ESI) m/z 143.3 [M+H]+.

### Step 4: Preparation of (5-fluoro-1-methyl-2-oxo-1,2-dihydropyridin-4-yl)carbamic chloride

To a cooled (0° C.) solution of ditrichloromethyl carbonate (0.6 g, 1.9 mmol, 0.3 eq) in DCM (10 mL) was added a solution of 4-amino-5-fluoro-1-methylpyridin-2(1H)-one (0.8 g, crude, 1 eq) in DCM and triethylamine (1.2 g, 11.5 mmol, 2.0 eq). The mixture was stirred at 0° C. for 30 min. The mixture was used next step without work up.

### Step 5: Preparation of Methyl 1-(3-(5-fluoro-1-methyl-2-oxo-1,2-dihydropyridin-4-yl)ureido)cyclopropane-1-carboxylate

To a solution of (5-fluoro-1-methyl-2-oxo-1,2-dihydropyridin-4-yl)carbamic chloride (1.2 g, crude) in DCM (10 mL) was added a solution of methyl 1-aminocyclopropane-1-carboxylate (1.0 g, 8.6 mmol, 1.5 eq) in DCM (2 mL) and triethylamine (1.7 g, 17 mmol, 3 eq). The mixture was stirred at 0° C. for 1 h to give a clear brown solution. The reaction mixture was concentrated under reduced pressure to give a residue. The residue was purified by reversed phase (TFA) then lyophilization to give a methyl 1-(3-(5-fluoro-1-methyl-2-oxo-1,2-dihydropyridin-4-yl)ureido)cyclopropane-1-carboxylate (1.0 g, 3.5 mmol, 62% yield) as a white solid. LCMS (ESI) m/z=283.7 [M+H]⁺.

### Step 6: 6-(5-fluoro-1-methyl-2-oxo-1,2-dihydropyridin-4-yl)-4,6-diazaspiro[2.4]heptane-5,7-dione

To a solution of methyl 1-(3-(5-fluoro-1-methyl-2-oxo-1,2-dihydropyridin-4-yl)ureido)cyclopropane-1-carboxylate (1.0 g, 3.5 mmol, 1 eq) in MeCN (20 mL) was added 1H,2H,3H,4H,6H,7H,8H-[1,3]diazino[1,2-a]pyrimidine (2.0 g, 14 mmol, 4 eq), the mixture was stirred at 25° C. for 1 h to give a yellow clean solution. The mixture was purified by reversed phase (TFA) then lyophilization to give 6-(5-fluoro-1-methyl-2-oxo-1,2-dihydropyridin-4-yl)-4,6-diazaspiro[2.4]heptane-5,7-dione (0.4 g, 1.4 mmol, 41% yield) as a white solid. LCMS (ESI) m/z=252.1 [M+H]⁺.

The following intermediates in Table 94 were prepared using the method described above for the synthesis of 6-(1-methyl-2-oxo-1,2-dihydropyridin-4-yl)-4,6-diazaspiro[2.4]heptane-5,7-dione, utilizing the appropriate starting materials and modifications.

TABLE 94

| Name | Structure | LCMS |
|---|---|---|
| 6-(1-methyl-2-oxo-1,2-dihydropyridin-4-yl)-4,6- diazaspiro[2.4]heptane-5,7-dione | 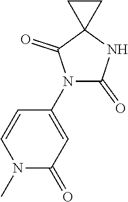 [SMILES: Cn1ccc(N2C(=O)NC3(CC3)C2=O)cc1=O] | 234.1 [M + H]⁺ |
| 6-(1-isopropyl-2-oxo-1,2-dihydropyridin-4-yl)-4,6- diazaspiro[2.4]heptane-5,7-dione | 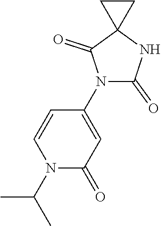 [SMILES: CC(C)n1ccc(N2C(=O)NC3(CC3)C2=O)cc1=O] | 262.1 [M + H]⁺ |
| 6-(2,2-difluorobenzo[d][1,3]dioxol-4-yl)-4,6- diazaspiro[2.4]heptane-5,7-dione | 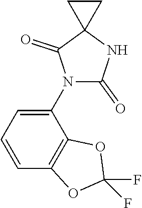 [SMILES: O=C1NC2(CC2)C(=O)N1c1cccc2c1OC(F)(F)O2] | 283.1 [M + H]⁺ |
| 6-(pyridin-3-yl)-4,6-diazaspiro[2.4]heptane-5,7-dione | 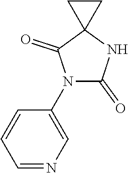 [SMILES: O=C1NC2(CC2)C(=O)N1c1cccnc1] | 204 [M + H]⁺ |
| 6-(1-(difluoromethyl)-2-oxo-1,2-dihydropyridin-4-yl)-4,6- diazaspiro[2.4]heptane-5,7-dione | 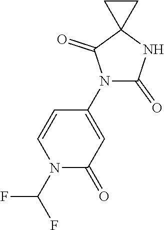 [SMILES: O=C1NC2(CC2)C(=O)N1c1ccn(C(F)F)c(=O)c1] | 270.2 [M + H]⁺ |

### Synthesis of 4-amino-1-(difluoromethyl)pyridin-2(1H)-one

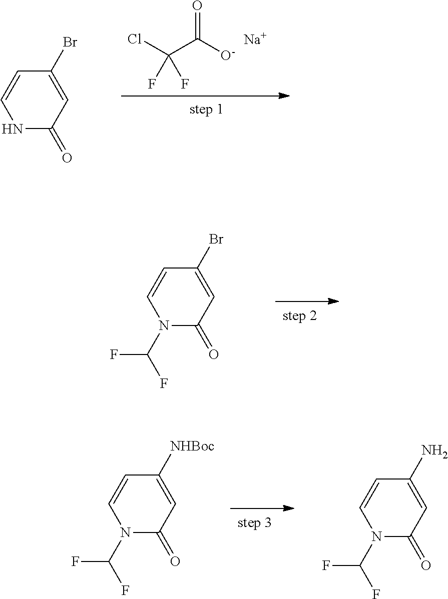 [SMILES: O=c1cc(Br)cc[nH]1 ; O=C([O-])C(F)(F)Cl ; [Na+] ; O=c1cc(Br)ccn1C(F)F ; CC(C)(C)OC(=O)Nc1ccn(C(F)F)c(=O)c1 ; Nc1ccn(C(F)F)c(=O)c1]

### Step 1: Preparation of 4-bromo-1-(difluoromethyl)pyridin-2(1H)-one

To a solution of 4-bromopyridin-2(1H)-one (5.0 g, 29 mmol, 1.0 eq) in DMF (50 mL) was added Cs₂CO₃ (14 g, 43 mmol, 1.5 eq) and sodium 2-chloro-2,2-difluoroacetate (4.4 g, 29 mmol, 1.0 eq). The mixture was stirred at 80° C. for 6 h. The reaction mixture was added to H₂O (100 mL) and extracted with EtOAc (100 mL×2). The combined organic layer was washed with brine (100 mL×2), dried over Na₂SO₄, filtered, and concentrated. The residue was purified by flash silica gel chromatography to give 4-bromo-1-(difluoromethyl)pyridin-2(1H)-one (1.0 g, 4.5 mmol, 15% yield) as a colorless oil. ¹H NMR (400 MHz, CDCl₃) δ 7.70-7.37 (m, 1H), 7.25 (d, J=7.6 Hz, 1H), 6.77 (s, 1H), 6.45-6.34 (m, 1H).

Step 2: Preparation of Tert-Butyl (1-(difluoromethyl)-2-oxo-1,2-dihydropyridin-4-yl)carbamate

A mixture of 4-bromo-1-(difluoromethyl)pyridin-2(1H)-one (0.9 g, 4.0 mmol, 1.0 eq) and tert-butyl carbamate (0.94 g, 8.0 mmol, 2.0 eq), [5-(diphenylphosphanyl)-9,9-dimethyl-9H-xanthen-4-yl]diphenylphosphane (0.46 g, 0.80 mmol, 0.2 eq), bis((1Z,4Z)-1,5-diphenylpenta-1,4-dien-3-one) (4Z)-1,5-diphenylpenta-1,4-dien-3-one dipalladium (0.37 g, 0.40 mmol, 0.1 eq) and Cs₂CO₃ (3.9 g, 12 mmol, 3 eq) in dioxane (30 mL) was degassed and purged with N₂ (3 times). The mixture was stirred at 100° C. for 12 h under N₂ atmosphere. The reaction mixture was filtered and concentrated under reduced pressure. The yellow residue was purified by column chromatography to give tert-butyl (1-(difluoromethyl)-2-oxo-1,2-dihydropyridin-4-yl)carbamate (0.6 g, 2.2 mmol, 57% yield) as a yellow solid. LCMS (ESI) m/z=261.0

### Step 3: Preparation of 4-amino-1-(difluoromethyl)pyridin-2(1H)-one

To a solution of tert-butyl (1-(difluoromethyl)-2-oxo-1,2-dihydropyridin-4-yl)carbamate (0.4 g, 1.5 mmol, 1.0 eq) in DCM (4 mL) was added HCl/dioxane (3.82 mL, 4 M), the mixture was stirred at 25° C. for 1 h. The mixture was concentrated in vacuo to give 4-amino-1-(difluoromethyl)pyridin-2(1H)-one (240 mg, crude) as a yellow solid. LCMS (ESI) m/z=161.0 [M+H]⁺.

### Synthesis of 5,5-dimethyl-3-(pyridin-3-yl)imidazolidine-2,4-dione

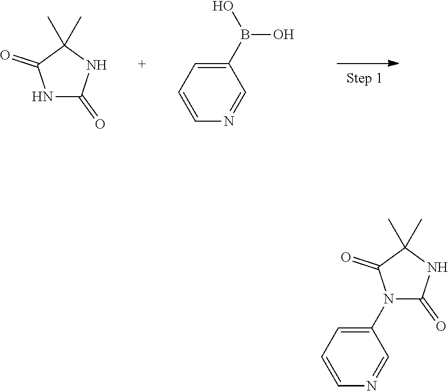

### Step 1: Preparation of 5,5-dimethyl-3-(pyridin-3-yl) imidazolidine-2,4-dione

To a solution of 5,5-dimethylimidazolidine-2,4-dione (1.4 g, 11 mmol, 1.4 eq) and copper diacetate (0.15 g, 0.81 mmol, 0.1 eq) in MeOH (40 mL) was added pyridin-3-ylboronic acid (1 g, 8.1 mmol, 1 eq) under an atmosphere of 02. The mixture was heated at 70° C. for 16 h. The solvent was then filtered through silica gel to remove copper, and the resulting cake was washed with methanol (200 mL). The reaction mixture was concentrated under reduced pressure to give a residue. The filtrate was purified by reversed phase (NH₃·H₂O) then lyophilized to give 5,5-dimethyl-3-(pyridin-3-yl) imidazolidine-2,4-dione (0.6 g, 2.9 mmol, 35% yield) as a yellow oil. LCMS (ESI) m/z=206.0 [M+H]⁺. ¹H NMR (400 MHz, CD₃OD) δ 8.67 (d, J=2.0 Hz, 1H), 8.57-8.51 (m, 1H), 8.00-7.92 (m, 1H), 7.53-7.57 (m, 1H), 1.52 (s, 6H).

The following intermediates in Table 95 were prepared using the method described above for the synthesis of 5,5-dimethyl-3-(pyridin-3-yl) imidazolidine-2,4-dione, utilizing the appropriate starting materials and modifications.

TABLE 95

| Name | Structure | LCMS | NMR |
|---|---|---|---|
| 7-(pyridin-3-yl)-5,7- diazaspiro[3.4]octane-6,8- dione | 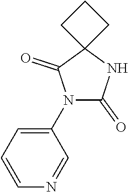 [SMILES: O=C1NC2(CCC2)C(=O)N1c1cccnc1] | 218.2 [M + H]⁺ | ¹H NMR (400 MHz, CD₃OD) δ 8.67 (d, J = 1.8 Hz, 1H), 8.53 (d, J = 4.4 Hz, 1H), 8.00-7.91 (m, 1H), 7.53-7.57 (m, 1H), 2.69-2.61 (m, 2H), 2.53-2.44 (m, 2H), 2.11-2.14 (m, 1H), 1.99-1.84 (m, 1H) |

### Synthesis of 4-((3S,6S,10aS)-6-amino-5-oxodecahydropyrrolo[1,2-a]azocine-3-carbonyl)-6-(6-(trifluoromethyl)pyridin-3-yl)-4,6-diazaspiro[2.4]heptane-5,7-dione

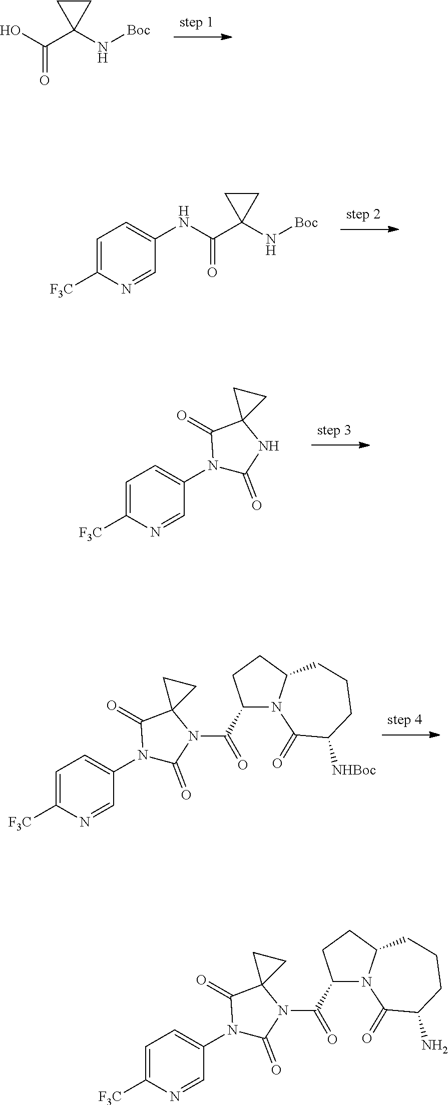 [SMILES: CC(C)(C)OC(=O)NC1(C(=O)O)CC1 ; CC(C)(C)OC(=O)NC1(C(=O)Nc2ccc(C(F)(F)F)nc2)CC1 ; O=C1NC2(CC2)C(=O)N1c1ccc(C(F)(F)F)nc1 ; CC(C)(C)OC(=O)N[C@H]1CCC[C@H]2CC[C@@H](C(=O)N3C(=O)N(c4ccc(C(F)(F)F)nc4)C(=O)C34CC4)N2C1=O ; N[C@H]1CCC[C@H]2CC[C@@H](C(=O)N3C(=O)N(c4ccc(C(F)(F)F)nc4)C(=O)C34CC4)N2C1=O]

### Step 1: Preparation of Tert-Butyl (1-((6-(trifluoromethyl)pyridin-3-yl)carbamoyl)cyclopropyl)carbamate

To a solution of 1-((tert-butoxycarbonyl)amino)cyclopropane-1-carboxylic acid (250 mg, 1.24 mmol, 1 eq) and EDC·HCl (283 mg, 1.48 mmol, 1.2 eq) in CH₂Cl₂ (60 mL) were respectively added 6-(trifluoromethyl)pyridin-3-amine (220 mg, 1.36 mmol, 1.1 eq) and DMAP (151 mg, 1.24 mmol, 1 eq). The resulting mixture was stirred at room temperature for 18 h. The solvent was removed under reduced pressure and the crude mixture was dissolved in DMSO and purified by reverse phase chromatography on a 100 g Cis cartridge eluting with 5-100% MeCN in water (with 0.1% formic acid). The pure fractions were concentrated were lyophilized to give tert-butyl (1-((6-(trifluoromethyl)pyridin-3-yl)carbamoyl)cyclopropyl)carbamate (320 mg, 74.7% yield) was obtained as a white solid LCMS (ESI) m/z=346.2 [M+H]⁺. ¹H NMR (400 MHz, CDCl₃) δ 8.96 (br. s., 1H), 8.64-8.52 (m, 1H), 8.37 (dd, J=8.5, 2.3 Hz, 1H), 7.66 (d, J=8.5 Hz, 1H), 5.18 (br. s., 1H), 1.73-1.66 (m, 2H), 1.50 (s, 9H), 1.20-1.12 (m, 2H).

The following intermediates in Table 96 were prepared using the method described above in Step 1 for the preparation of tert-butyl (1-((6-(trifluoromethyl)pyridin-3-yl)carbamoyl)cyclopropyl)carbamate utilizing the appropriate starting materials and modifications.

TABLE 96

| Name | Structure | LCMS |
|---|---|---|
| tert-butyl (1-((5-methylpyridin-3- yl)carbamoyl)cyclopropyl)carbamate | 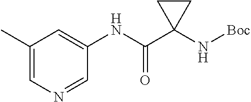 [SMILES: Cc1cncc(NC(=O)C2(NC(=O)OC(C)(C)C)CC2)c1] | 292.2 [M + H]⁺ |
| tert-butyl (1-((6-methylpyridin-3- yl)carbamoyl)cyclopropyl)carbamate | 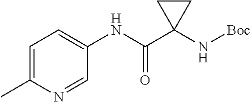 [SMILES: Cc1ccc(NC(=O)C2(NC(=O)OC(C)(C)C)CC2)cn1] | 292.2 [M + H]⁺ |
| tert-butyl (1-((4-methylpyridin-3- yl)carbamoyl)cyclopropyl)carbamate | 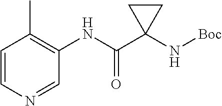 [SMILES: Cc1ccncc1NC(=O)C1(NC(=O)OC(C)(C)C)CC1] | 292.2 [M + H]⁺ |
| tert-butyl (1-((6-methoxypyridin-3- yl)carbamoyl)cyclopropyl)carbamate | 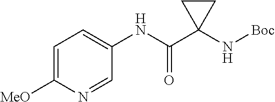 [SMILES: COc1ccc(NC(=O)C2(NC(=O)OC(C)(C)C)CC2)cn1] | 308.2 [M + H]⁺ |
| tert-butyl (1-((5-(trifluoromethyl)pyridin-3- yl)carbamoyl)cyclopropyl)carbamate | 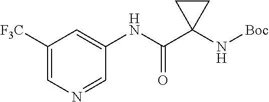 [SMILES: CC(C)(C)OC(=O)NC1(C(=O)Nc2cncc(C(F)(F)F)c2)CC1] | 346.2 [M + H]⁺ |
| tert-butyl (1-((5-methoxypyridin-3- yl)carbamoyl)cyclopropyl)carbamate | 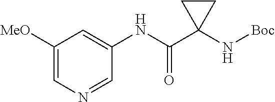 [SMILES: COc1cncc(NC(=O)C2(NC(=O)OC(C)(C)C)CC2)c1] | 308.4 [M + H]⁺ |
| tert-butyl (1-((6-(difluoromethyl)pyridin-3- yl)carbamoyl)cyclopropyl)carbamate | 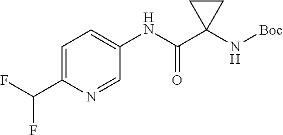 [SMILES: CC(C)(C)OC(=O)NC1(C(=O)Nc2ccc(C(F)F)nc2)CC1] | 328.2 [M + H]⁺ |
| tert-butyl (1-((5-(difluoromethyl)pyridin-3- yl)carbamoyl)cyclopropyl)carbamate | 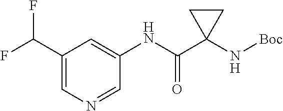 [SMILES: CC(C)(C)OC(=O)NC1(C(=O)Nc2cncc(C(F)F)c2)CC1] | 328.2 [M + H]⁺ |

### Step 2: Preparation of 6-(6-(trifluoromethyl)pyridin-3-yl)-4,6-diazaspiro[2.4]heptane-5,7-dione

To a stirred solution of tert-butyl (1-((6-(trifluoromethyl)pyridin-3-yl)carbamoyl)cyclopropyl)carbamate (115 mg, 333 μmol, 1 eq) and DTBMP (143 mg, 699 μmol, 2.1 eq) in CH₂Cl₂ (5 mL) at 0° C. was added Tf₂O (197 mg, 699 μmol, 2.1 eq) under nitrogen. The reaction mixture was stirred for 1 h. CH₂Cl₂ was removed with a nitrogen stream and the resulting mixture was purified by reverse phase chromatography on a 50 g C₁₈ cartridge eluting with 5-100% MeCN in water (with 0.1% formic acid). The pure fractions lyophilized to give 6-(6-(trifluoromethyl)pyridin-3-yl)-4,6-diazaspiro[2.4]heptane-5,7-dione (50.0 mg, 55.3% yield) a white solid LCMS (ESI) m/z=272.2 [M+H]⁺.

The following intermediates in Table 97 were prepared using the method described above in Step 2 for the preparation of 6-(6-(trifluoromethyl)pyridin-3-yl)-4,6-diazaspiro[2.4]heptane-5,7-dione utilizing the appropriate starting materials and modifications.

TABLE 97

| Name | Structure | LCMS |
|---|---|---|
| 6-(5-methylpyridin-3-yl)-4,6- diazaspiro[2.4]heptane-5,7-dione | 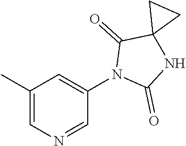 [SMILES: Cc1cncc(N2C(=O)NC3(CC3)C2=O)c1] | 218.1 [M + H]⁺ |
| 6-(6-methylpyridin-3-yl)-4,6- diazaspiro[2.4]heptane-5,7-dione | 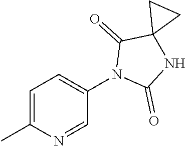 [SMILES: Cc1ccc(N2C(=O)NC3(CC3)C2=O)cn1] | 218.1 [M + H]⁺ |
| 6-(4-methylpyridin-3-yl)-4,6- diazaspiro[2.4]heptane-5,7-dione | 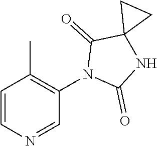 [SMILES: Cc1ccncc1N1C(=O)NC2(CC2)C1=O] | 218.1 [M + H]⁺ |
| 6-(6-methoxypyridin-3-yl)-4,6- diazaspiro[2.4]heptane-5,7-dione | 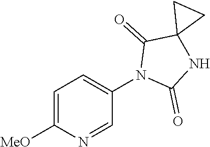 [SMILES: COc1ccc(N2C(=O)NC3(CC3)C2=O)cn1] | 234.2 [M + H]⁺ |
| 6-(5-(trifluoromethyl)pyridin-3-yl)-4,6- diazaspiro[2.4]heptane-5,7-dione | 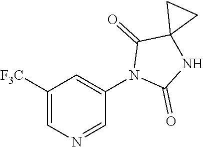 [SMILES: O=C1NC2(CC2)C(=O)N1c1cncc(C(F)(F)F)c1] | 272.2 [M + H]⁺ |
| 6-(5-methoxypyridin-3-yl)-4,6- diazaspiro[2.4]heptane-5,7-dione | 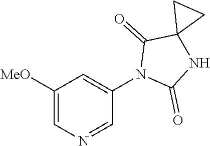 [SMILES: COc1cncc(N2C(=O)NC3(CC3)C2=O)c1] | 234.2 [M + H]⁺ |
| 6-(6-(difluoromethyl)pyridin-3-yl)-4,6- diazaspiro[2.4]heptane-5,7-dione | 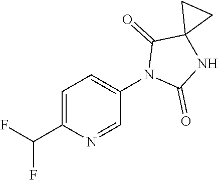 [SMILES: O=C1NC2(CC2)C(=O)N1c1ccc(C(F)F)nc1] | 254.2 [M + H]⁺ |
| 6-(5-(difluoromethyl)pyridin-3-yl)-4,6- diazaspiro[2.4]heptane-5,7-dione | 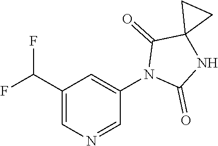 [SMILES: O=C1NC2(CC2)C(=O)N1c1cncc(C(F)F)c1] | 254.2 [M + H]⁺ |

### Step 3: Preparation of Tert-Butyl ((3S,6S,10aS)-3-(5,7-dioxo-6-(6-(trifluoromethyl)pyridin-3-yl)-4,6-diazaspiro[2.4]heptane-4-carbonyl)-5-oxodecahydropyrrolo[1,2-a]azocin-6-yl)carbamate

To a stirred solution of 6-(6-(trifluoromethyl)pyridin-3-yl)-4,6-diazaspiro[2.4]heptane-5,7-dione (30 mg, 110 μmol, 1 eq) and DIPEA (71.0 mg, 550 μmol, 5.0 eq) in N,N-dimethylformamide (6 mL) were respectively added (3S,6S,10aS)-6-((tert-butoxycarbonyl)amino)-5-oxodecahydropyrrolo[1,2-a]azocine-3-carboxylic acid (35.9 mg, 110 μmol, 1 eq) and PyBOP (68.6 mg, 132 μmol, 1.2 eq) under nitrogen. The reaction mixture was stirred at room temperature for 24 h and 3 h at 50° C. Then, the crude mixture was purified by reverse phase chromatography on a 50 g C₁₈ cartridge eluting with 5-100% MeCN in basic water (10 mM NH₄HCO₃ (pH=10) buffer). The pure fractions were lyophilized to yield tert-butyl ((3S,6S,10aS)-3-(5,7-dioxo-6-(6-(trifluoromethyl)pyridin-3-yl)-4,6-diazaspiro[2.4]heptane-4-carbonyl)-5-oxodecahydropyrrolo[1,2-a]azocin-6-yl)carbamate (34.6 mg, 54%) as a white solid. LCMS (ESI) m/z=480.4 [(M-Boc)+2H]⁺.

The following intermediates in Table 98 were prepared using the method described above in Step 3 for the preparation of tert-butyl ((3S,6S,10aS)-3-(5,7-dioxo-6-(6-(trifluoromethyl)pyridin-3-yl)-4,6-diazaspiro[2.4]heptane-4-carbonyl)-5-oxodecahydropyrrolo[1,2-a]azocin-6-yl)carbamate utilizing the appropriate starting materials and modifications.

TABLE 98

| Name | Structure | LCMS |
|---|---|---|
| tert-butyl ((3S,6S,10aS)-3-(6-(5- methylpyridin-3-yl)-5,7-dioxo-4,6- diazaspiro[2.4]heptane-4-carbonyl)-5- oxodecahydropyrrolo[1,2-a]azocin-6- yl)carbamate | 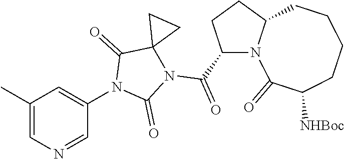 [SMILES: Cc1cncc(N2C(=O)N(C(=O)[C@@H]3CC[C@@H]4CCCC[C@H](NC(=O)OC(C)(C)C)C(=O)N43)C3(CC3)C2=O)c1] | 526.3 [M + H]⁺ |
| tert-butyl ((3S,6S,10aS)-3-(6-(6- methylpyridin-3-yl)-5,7-dioxo-4,6- diazaspiro[2.4]heptane-4-carbonyl)-5- oxodecahydropyrrolo[1,2-a]azocin-6- yl)carbamate | 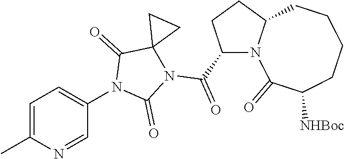 [SMILES: Cc1ccc(N2C(=O)N(C(=O)[C@@H]3CC[C@@H]4CCCC[C@H](NC(=O)OC(C)(C)C)C(=O)N43)C3(CC3)C2=O)cn1] | 526.3 [M + H]⁺ |
| tert-butyl ((3S,6S,10aS)-3-(6-(4- methylpyridin-3-yl)-5,7-dioxo-4,6- diazaspiro[2.4]heptane-4-carbonyl)-5- oxodecahydropyrrolo[1,2-a]azocin-6- yl)carbamate | 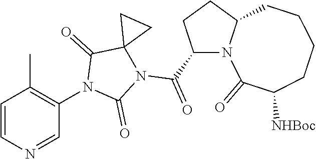 [SMILES: Cc1ccncc1N1C(=O)N(C(=O)[C@@H]2CC[C@@H]3CCCC[C@H](NC(=O)OC(C)(C)C)C(=O)N32)C2(CC2)C1=O] | 526.3 [M + H]⁺ |
| tert-butyl ((3S,6S,10aS)-3-(6-(6- methoxypyridin-3-yl)-5,7-dioxo-4,6- diazaspiro[2.4]heptane-4-carbonyl)-5- oxodecahydropyrrolo[1,2-a]azocin-6- yl)carbamate | 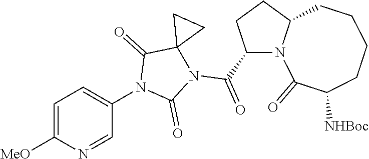 [SMILES: COc1ccc(N2C(=O)N(C(=O)[C@@H]3CC[C@@H]4CCCC[C@H](NC(=O)OC(C)(C)C)C(=O)N43)C3(CC3)C2=O)cn1] | 542.4 [M + H]⁺ |
| tert-butyl ((3S,6S,10aS)-3-(5,7-dioxo-6-(5- (trifluoromethyl)pyridin-3-yl)-4,6- diazaspiro[2.4]heptane-4-carbonyl)-5- oxodecahydropyrrolo[1,2-a]azocin-6- yl)carbamate | 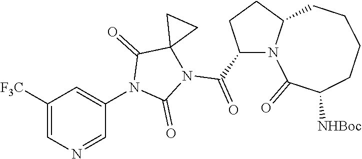 [SMILES: CC(C)(C)OC(=O)N[C@H]1CCCC[C@H]2CC[C@@H](C(=O)N3C(=O)N(c4cncc(C(F)(F)F)c4)C(=O)C34CC4)N2C1=O] | 580.4 [M + H]⁺ |
| tert-butyl ((3S,6S,10aS)-3-(6-(5- methoxypyridin-3-yl)-5,7-dioxo-4,6- diazaspiro[2.4]heptane-4-carbonyl)-5- oxodecahydropyrrolo[1,2-a]azocin-6- yl)carbamate | 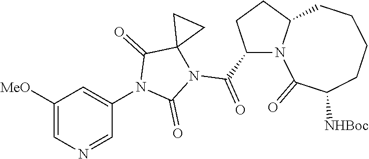 [SMILES: COc1cncc(N2C(=O)N(C(=O)[C@@H]3CC[C@@H]4CCCC[C@H](NC(=O)OC(C)(C)C)C(=O)N43)C3(CC3)C2=O)c1] | 542.4 [M + H]⁺ |
| tert-butyl ((3S,6S,10aS)-3-(6-(6- (difluoromethyl)pyridin-3-yl)-5,7-dioxo-4,6- diazaspiro[2.4]heptane-4-carbonyl)-5- oxodecahydropyrrolo[1,2-a]azocin-6- yl)carbamate | 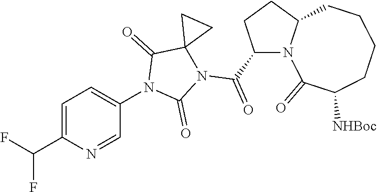 [SMILES: CC(C)(C)OC(=O)N[C@H]1CCCC[C@H]2CC[C@@H](C(=O)N3C(=O)N(c4ccc(C(F)F)nc4)C(=O)C34CC4)N2C1=O] | 562.4 [M + H]⁺ |
| tert-butyl ((3S,6S,10aS)-3-(6-(5- (difluoromethyl)pyridin-3-yl)-5,7-dioxo-4,6- diazaspiro[2.4]heptane-4-carbonyl)-5- oxodecahydropyrrolo[1,2-a]azocin-6- yl)carbamate | 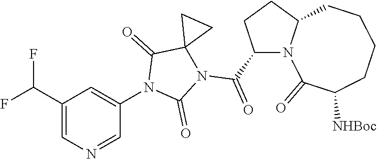 [SMILES: CC(C)(C)OC(=O)N[C@H]1CCCC[C@H]2CC[C@@H](C(=O)N3C(=O)N(c4cncc(C(F)F)c4)C(=O)C34CC4)N2C1=O] | 562.4 [M + H]⁺ |

### Step 4: Preparation of 4-((3S,6S,10aS)-6-amino-5-oxodecahydropyrrolo[1,2-a]azocine-3-carbonyl)-6-(6-(trifluoromethyl)pyridin-3-yl)-4,6-diazaspiro[2.4]heptane-5,7-dione

To a stirred solution of tert-butyl ((3S,6S,10aS)-3-(5,7-dioxo-6-(6-(trifluoromethyl)pyridin-3-yl)-4,6-diazaspiro[2.4]heptane-4-carbonyl)-5-oxodecahydropyrrolo[1,2-a]azocin-6-yl)carbamate (21.6 mg, 37.4 μmol, 1 eq) in CH₂+₂(1.5 mL) was added TFA (500 μL). The resulting yellow solution was stirred for 1 h at room temperature. The reaction was concentrated under reduced pressure to dryness to give the TFA salt of 4-((3S,6S,10aS)-6-amino-5-oxodecahydropyrrolo[1,2-a]azocine-3-carbonyl)-6-(6-(trifluoromethyl)pyridin-3-yl)-4,6-diazaspiro[2.4]heptane-5,7-dione (22.1 mg, 100%) that was used directly in the next step. LCMS (ESI) m/z 480.2 [M+H].

The following intermediates in Table 99 were prepared using the method described above in Step 4 for the preparation of 4-((3S,6S,10aS)-6-amino-5-oxodecahydropyrrolo[1,2-a]azocine-3-carbonyl)-6-(6-(trifluoromethyl)pyridin-3-yl)-4,6-diazaspiro[2.4]heptane-5,7-dione utilizing the appropriate starting materials and

TABLE 99

| Name | Structure | LCMS |
|---|---|---|
| 4-((3S,6S,10aS)-6-amino-5- oxodecahydropyrrolo[1,2-a]azocine-3-carbonyl)- 6-(5-methylpyridin-3-yl)-4,6- diazaspiro[2.4]heptane-5,7-dione (TFA salt) | 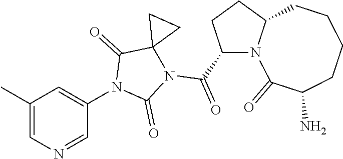 [SMILES: Cc1cncc(N2C(=O)N(C(=O)[C@@H]3CC[C@@H]4CCCC[C@H](N)C(=O)N43)C3(CC3)C2=O)c1] | 426.2 [M + H]⁺ |
| 4-((3S,6S,10aS)-6-amino-5- oxodecahydropyrrolo[1,2-a]azocine-3-carbonyl)- 6-(6-methylpyridin-3-yl)-4,6- diazaspiro[2.4]heptane-5,7-dione (TFA salt) | 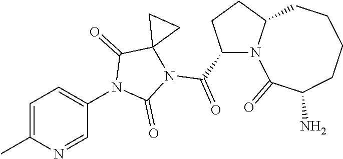 [SMILES: Cc1ccc(N2C(=O)N(C(=O)[C@@H]3CC[C@@H]4CCCC[C@H](N)C(=O)N43)C3(CC3)C2=O)cn1] | 426.2 [M + H]⁺ |
| 4-((3S,6S,10aS)-6-amino-5- oxodecahydropyrrolo[1,2-a]azocine-3-carbonyl)- 6-(4-methylpyridin-3-yl)-4,6- diazaspiro[2.4]heptane-5,7-dione (TFA salt) | 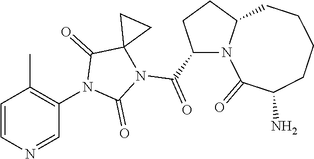 [SMILES: Cc1ccncc1N1C(=O)N(C(=O)[C@@H]2CC[C@@H]3CCCC[C@H](N)C(=O)N32)C2(CC2)C1=O] | 426.2 [M + H]⁺ |
| 4-((3S,6S,10aS)-6-amino-5- oxodecahydropyrrolo[1,2-a]azocine-3-carbonyl)- 6-(6-methoxypyridin-3-yl)-4,6- diazaspiro[2.4]heptane-5,7-dione (TFA salt) | 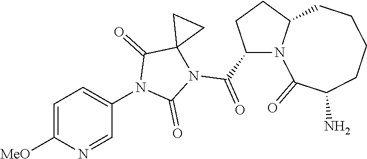 [SMILES: COc1ccc(N2C(=O)N(C(=O)[C@@H]3CC[C@@H]4CCCC[C@H](N)C(=O)N43)C3(CC3)C2=O)cn1] | 442.2 [M + H]⁺ |
| 4-((3S,6S,10aS)-6-amino-5- oxodecahydropyrrolo[1,2-a]azocine-3-carbonyl)- 6-(5-(trifluoromethyl)pyridin-3-yl)-4,6- diazaspiro[2.4]heptane-5,7-dione (TFA salt) | 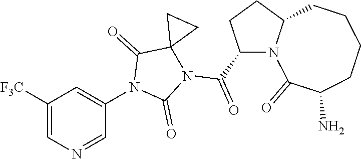 [SMILES: N[C@H]1CCCC[C@H]2CC[C@@H](C(=O)N3C(=O)N(c4cncc(C(F)(F)F)c4)C(=O)C34CC4)N2C1=O] | 480.2 [M + H]⁺ |
| 4-((3S,6S,10aS)-6-amino-5- oxodecahydropyrrolo[1,2-a]azocine-3-carbonyl)- 6-(5-methoxypyridin-3-yl)-4,6- diazaspiro[2.4]heptane-5,7-dione (TFA salt) | 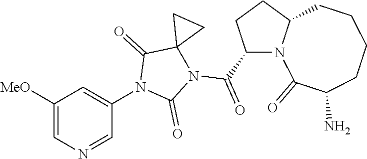 [SMILES: COc1cncc(N2C(=O)N(C(=O)[C@@H]3CC[C@@H]4CCCC[C@H](N)C(=O)N43)C3(CC3)C2=O)c1] | 221.6 [M + 2H]²⁺ |
| 4-((3S,6S,10aS)-6-amino-5- oxodecahydropyrrolo[1,2-a]azocine-3-carbonyl)- 6-(6-(difluoromethyl)pyridin-3-yl)-4,6- diazaspiro[2.4]heptane-5,7-dione (TFA salt) | 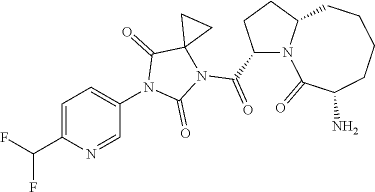 [SMILES: N[C@H]1CCCC[C@H]2CC[C@@H](C(=O)N3C(=O)N(c4ccc(C(F)F)nc4)C(=O)C34CC4)N2C1=O] | 462.2 [M + H]⁺ |
| 4-((3S,6S,10aS)-6-amino-5- oxodecahydropyrrolo[1,2-a]azocine-3-carbonyl)- 6-(5-(difluoromethyl)pyridin-3-yl)-4,6- diazaspiro[2.4]heptane-5,7-dione (TFA salt) | 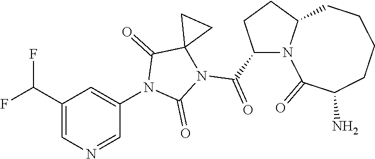 [SMILES: N[C@H]1CCCC[C@H]2CC[C@@H](C(=O)N3C(=O)N(c4cncc(C(F)F)c4)C(=O)C34CC4)N2C1=O] | 462.2 [M + H]⁺ |

### Synthesis of tert-butyl ((3S,6S,10aS)-3-((6R,7S)-7-cyano-6-(3-methoxyphenyl)-4-azaspiro[2.4]heptane-4-carbonyl)-5-oxodecahydropyrrolo[1,2-a]azocin-6-yl)carbamate and tert-butyl ((3S,6S,10aS)-3-((6S,7R)-7-cyano-6-(3-methoxyphenyl)-4-azaspiro[2.4]heptane-4-carbonyl)-5-oxodecahydropyrrolo[1,2-a]azocin-6-yl)carbamate

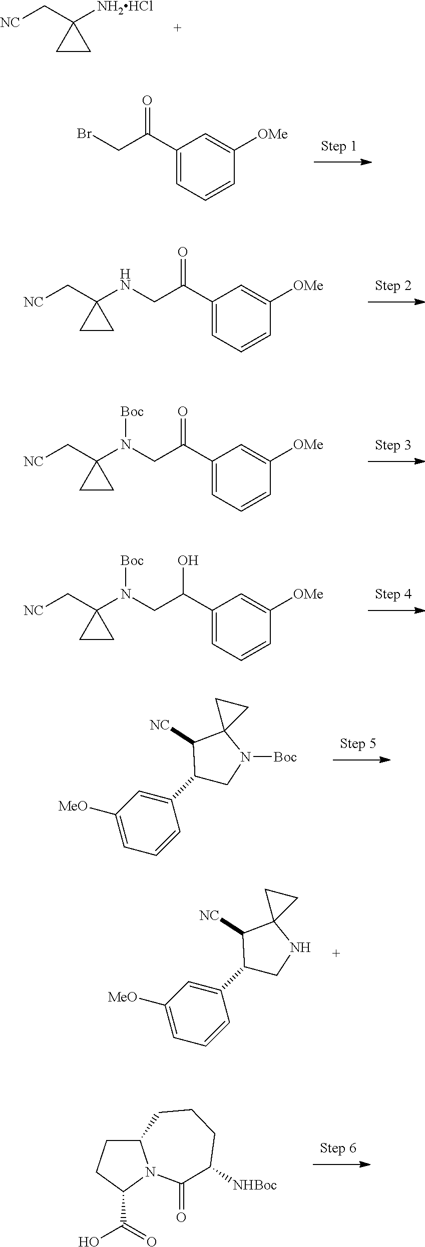

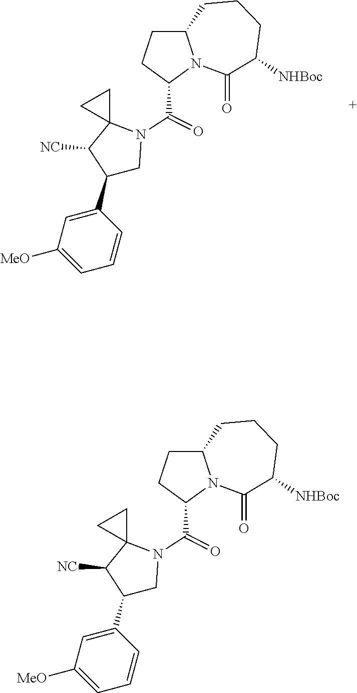 [SMILES: COc1cccc([C@@H]2CN(C(=O)[C@@H]3CC[C@@H]4CCC[C@H](NC(=O)OC(C)(C)C)C(=O)N43)C3(CC3)[C@H]2C#N)c1 ; COc1cccc([C@H]2CN(C(=O)[C@@H]3CC[C@@H]4CCC[C@H](NC(=O)OC(C)(C)C)C(=O)N43)C3(CC3)[C@@H]2C#N)c1]

### Step 1: Preparation of 2-(1-((2-(3-methoxyphenyl)-2-oxoethyl)amino)cyclopropyl)acetonitrile

To a solution of 2-(1-aminocyclopropyl)acetonitrile hydrochloride (367 mg, 3.82 mmol, 1.0 eq) in DMF (10 mL) were added 2-bromo-1-(3-methoxyphenyl)ethan-1-one (918 mg, 4.01 mmol, 1.05 eq) and NaHCO₃(801 mg, 9.54 mmol, 2.5 eq). The mixture was stirred at room temperature for 20 h. The reaction was diluted with MeCN and filtered. The filtrate was concentrated under reduced pressure. The crude solution was purified by reverse phase chromatography on a C₁₈ cartridge eluting with 5-60% MeCN in basic water (10 mM NH₄HCO₃ (pH=10) buffer). The pure fractions were concentrated under reduced pressure to give 2-(1-((2-(3-methoxyphenyl)-2-oxoethyl)amino)cyclopropyl)acetonitrile (522 mg, 55.9%) as an orange oil. LCMS (ESI) m/z=245.1 [M+H]⁺. ¹H NMR (400 MHz, CDCl₃) δ 7.53-7.51 (m, 1H), 7.49-7.46 (m, 1H), 7.38 (t, J=7.9 Hz, 1H), 7.16-7.10 (m, 1H), 4.25 (s, 2H), 3.86 (s, 3H), 2.58 (s, 2H), 0.89-0.84 (m, 2H), 0.74-0.70 (m, 2H).

The following intermediates in Table 100 were prepared using the method described above in Step 1 for the preparation of 2-(1-((2-(3-methoxyphenyl)-2-oxoethyl)amino)cyclopropyl)acetonitrile utilizing the appropriate starting materials and modifications.

TABLE 100

| Name | Structure | LCMS |
|---|---|---|
| 2-(1-((2-(3-fluorophenyl)-2- oxoethyl)amino)cyclopropyl)acetonitrile | 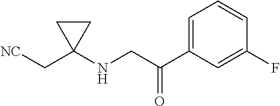 [SMILES: N#CCC1(NCC(=O)c2cccc(F)c2)CC1] | 233.2 [M + H]⁺ |
| 2-(1-((2-(3-(difluoromethoxy)phenyl)-2- oxoethyl)amino)cyclopropyl)acetonitrile | 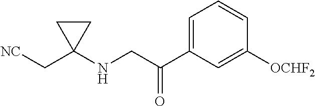 [SMILES: N#CCC1(NCC(=O)c2cccc(OC(F)F)c2)CC1] | 281.2 [M + H]⁺ |
| 3-((1- (cyanomethyl)cyclopropyl)glycyl)benzonitrile | 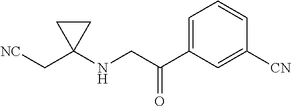 [SMILES: N#CCC1(NCC(=O)c2cccc(C#N)c2)CC1] | 240.2 [M + H]⁺ |
| 2-(1-((2-oxo-2-(m- tolyl)ethyl)amino)cyclopropyl)acetonitrile | 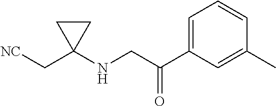 [SMILES: Cc1cccc(C(=O)CNC2(CC#N)CC2)c1] | 229.2 [M + H]⁺ |
| 2-(1-((2-(3-(difluoromethyl)phenyl)-2- oxoethyl)amino)cyclopropyl)acetonitrile | 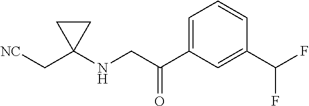 [SMILES: N#CCC1(NCC(=O)c2cccc(C(F)F)c2)CC1] | 265.2 [M + H]⁺ |

### Step 2: Preparation of Tert-Butyl (1-(cyanomethyl)cyclopropyl)(2-(3-methoxyphenyl)-2-oxoethyl)carbamate

To a solution of 2-(1-((2-(3-methoxyphenyl)-2-oxoethyl)amino)cyclopropyl)acetonitrile (522 mg, 2.13 mmol, 1 eq) in THF (1 mL) were added water (1 mL) and NaHCO₃(178 mg, 2.13 mmol, 1.0 eq) followed by di-tert-butyl dicarbonate (929 mg, 4.26 mmol, 2 eq) at 0° C. The reaction was warmed up to room temperature and stirred for 48 h at room temperature. The reaction was diluted with MeCN and filtered. The filtrate was concentrated under reduced pressure. The crude residue was purified by reverse phase chromatography on a C₁₈ cartridge eluting with 5-80% MeCN in water (with 0.1% formic acid) to give tert-butyl (1-(cyanomethyl)cyclopropyl)(2-(3-methoxyphenyl)-2-oxoethyl)carbamate (400 mg, 54.5%) as a sticky oil. LCMS (ESI) m/z=245.2 [(M-Boc)+2H]⁺.

The following intermediates in Table 101 were prepared using the method described above in Step 2 for the preparation of tert-butyl (1-(cyanomethyl)cyclopropyl)(2-(3-methoxyphenyl)-2-oxoethyl)carbamate utilizing the appropriate starting materials and modifications.

TABLE 101

| Name | Structure | LCMS |
|---|---|---|
| tert-butyl (1-(cyanomethyl)cyclopropyl)(2- (3-fluorophenyl)-2-oxoethyl)carbamate | 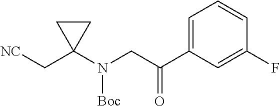 [SMILES: CC(C)(C)OC(=O)N(CC(=O)c1cccc(F)c1)C1(CC#N)CC1] | 277.2 [(M—tBu) + 2H]⁺ |
| tert-butyl (1-(cyanomethyl)cyclopropyl)(2- (3-(difluoromethoxy)phenyl)-2- oxoethyl)carbamate | 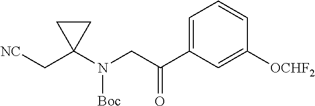 [SMILES: CC(C)(C)OC(=O)N(CC(=O)c1cccc(OC(F)F)c1)C1(CC#N)CC1] | 281.2 [(M—Boc) + 2H)⁺ |
| tert-butyl (1-(cyanomethyl)cyclopropyl)(2- (3-cyanophenyl)-2-oxoethyl)carbamate | 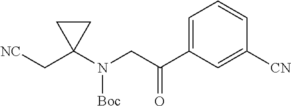 [SMILES: CC(C)(C)OC(=O)N(CC(=O)c1cccc(C#N)c1)C1(CC#N)CC1] | 240.2 [(M—Boc) + 2H]⁺ |
| tert-butyl (1-(cyanomethyl)cyclopropyl)(2- oxo-2-(m-tolyl)ethyl)carbamate | 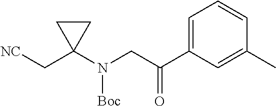 [SMILES: Cc1cccc(C(=O)CN(C(=O)OC(C)(C)C)C2(CC#N)CC2)c1] | 273.2 [(M—tBu) + 2H]⁺ |
| tert-butyl (1-(cyanomethyl)cyclopropyl)(2- (3-(difluoromethyl)phenyl)-2- oxoethyl)carbamate | 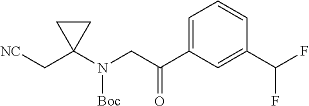 [SMILES: CC(C)(C)OC(=O)N(CC(=O)c1cccc(C(F)F)c1)C1(CC#N)CC1] | 309 [(M—tBu) + 2H]⁺ |

### Step 3: Preparation of Tert-Butyl (1-(cyanomethyl)cyclopropyl)(2-hydroxy-2-(3-methoxyphenyl)ethyl)carbamate

To a solution of tert-butyl (1-(cyanomethyl)cyclopropyl)(2-(3-methoxyphenyl)-2-oxoethyl)carbamate (193 mg, 0.5603 mmol, 1 eq) in methanol (10 mL) at 0° C. was added NaBH₄ (21.1 mg, 0.5603 mmol, 1.0 eq). The reaction was stirred for 2 h at room temperature. The reaction was quenched with 1 N HCl (1 mL) and concentrated under reduced pressure. The crude residue was purified by reverse phase chromatography on a Cis cartridge eluting with 5-60% MeCN in water (with 0.1% formic acid) to give tert-butyl (1-(cyanomethyl)cyclopropyl)(2-hydroxy-2-(3-methoxyphenyl)ethyl)carbamate (144 mg, 74.2%) as a thick clear oil. ¹H NMR (400 MHz, DMSO-d₆) δ 7.26 (t, J=7.8 Hz, 1H), 6.91-6.80 (m, 3H), 5.56-5.48 (m, 1H), 4.88-4.71 (m, 1H), 3.75 (s, 3H), 3.40-3.30 (m, 1H), 3.19-3.10 (m, 1H), 3.04-2.92 (m, 1H), 2.73-2.61 (m, 1H), 1.46 (s, 9H), 1.32-1.20 (m, 1H), 0.84-0.62 (m, 3H).

The following intermediates in Table 102 were prepared using the method described above in Step 3 for the preparation of tert-butyl (1-(cyanomethyl)cyclopropyl)(2-hydroxy-2-(3-methoxyphenyl)ethyl)carbamate utilizing the appropriate starting materials and

TABLE 102

| Name | Structure | LCMS | NMR |
|---|---|---|---|
| tert-butyl (1- (cyanomethyl)cyclopropyl)(2- (3-fluorophenyl)-2- hydroxyethyl)carbamate | 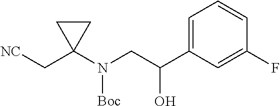 [SMILES: CC(C)(C)OC(=O)N(CC(O)c1cccc(F)c1)C1(CC#N)CC1] | 261.2 [(M—tBu − OH) + 2H]⁺ | |
| tert-butyl (1- (cyanomethyl)cyclopropyl)(2- (3-(difluoromethoxy)phenyl)- 2-hydroxyethyl)carbamate | 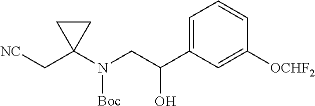 [SMILES: CC(C)(C)OC(=O)N(CC(O)c1cccc(OC(F)F)c1)C1(CC#N)CC1] | | ¹H NMR (400 MHz, CDCl₃) δ 7.35 (t, J = 7.8 Hz, 1H), 7.22- 7.18 (m, 1H), 7.17- 7.13 (m, 1H), 7.07- 7.02 (m, 1H), 6.53 (t, J = 73.8 Hz, 1H), 5.00- |
| | | | 4.91 (m, 1H), 3.63- |
| | | | 3.42 (m, 2H), 2.83- |
| | | | 2.45 (m, 1H), 1.65- |
| | | | 1.56 (m, 2H), 1.54 (s, |
| | | | 9H), 0.90-0.78 (m, |
| | | | 4H). |
| tert-butyl (1- (cyanomethyl)cyclopropyl)(2- (3-cyanophenyl)-2- hydroxyethyl)carbamate | 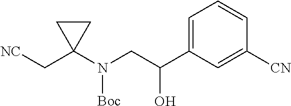 [SMILES: CC(C)(C)OC(=O)N(CC(O)c1cccc(C#N)c1)C1(CC#N)CC1] | | ¹H NMR (400 MHz, CDCl₃) δ 7.72-7.68 (m, 1H), 7.64-7.61 (m, 1H), 7.60-7.56 (m, 1H), 7.47 (t, J = 7.7 Hz, 1H), 5.03- 4.93 (m, 1H), 4.68- |
| | | | 4.25 (m, 1H), 3.61- |
| | | | 3.40 (m, 2H), 2.89- |
| | | | 2.37 (m, 2H), 1.54 (s, |
| | | | 9H), 1.02-0.89 (m, |
| | | | 4H). |
| tert-butyl (1- (cyanomethyl)cyclopropyl)(2- hydroxy-2-(m- tolyl)ethyl)carbamate | 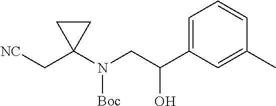 [SMILES: Cc1cccc(C(O)CN(C(=O)OC(C)(C)C)C2(CC#N)CC2)c1] | | ¹H NMR (400 MHz, CDCl₃) δ 7.26-7.22 (m, 1H), 7.19-7.07 (m, 3H), 4.95-4.87 (m, 1H), 4.31-3.97 (m, 1H), 3.62-3.41 (m, 2H), 2.88-2.49 |
| | | | (m, 2H), 2.36 (s, 3H), |
| | | | 1.54 (s, 9H), 1.03- |
| | | | 0.87 (m, 4H) |
| tert-butyl (1- (cyanomethyl)cyclopropyl)(2- (3-(difluoromethyl)phenyl)- 2-hydroxyethyl)carbamate | 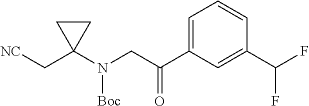 [SMILES: CC(C)(C)OC(=O)N(CC(=O)c1cccc(C(F)F)c1)C1(CC#N)CC1] | 293.2 [(M—tBu − OH) + 2H]⁺ | |

### Step 4: Preparation of Racemic-tert-butyl (6S,7R)-7-cyano-6-(3-methoxyphenyl)-4-azaspiro[2.4]heptane-4-carboxylate

To a solution of tert-butyl (1-(cyanomethyl)cyclopropyl)(2-hydroxy-2-(3-methoxyphenyl)ethyl)carbamate (115 mg, 0.3319 mmol, 1 eq) in THF (10 mL) at −15° C. were added diethyl phosphorochloridate (50.3 μL, 0.3484 mmol, 1.05 eq) followed by dropwise addition of 1 M LiHMDS in THF (829 μL, 0.8297 mmol, 2.5 eq). The reaction was stirred at −15° C. for 2 h. The reaction was quenched with 1 N HCl (1 mL) and concentrated under reduced pressure. The crude residue was purified by reverse phase chromatography on a C₁₈ cartridge eluting with 5-70% MeCN in water (with 0.1% formic acid) to give racemic-tert-butyl (6S,7R)-7-cyano-6-(3-methoxyphenyl)-4-azaspiro[2.4]heptane-4-carboxylate (50.0 mg, 46.2%) as a white solid. LCMS (ESI) m/z=273.2 [(M-t-Bu)+2H]⁺. ¹H NMR (400 MHz, C₆D₆) δ 7.03-6.98 (m, 1H), 6.66-6.61 (m, 2H), 6.53-6.49 (m, 1H), 3.72-3.62 (m, 1H), 3.29 (s, 3H), 3.25-3.18 (m, 1H), 3.04-2.96 (m, 1H), 2.71 (d, J=10.5 Hz, 1H), 2.38-1.93 (m, 1H), 1.75-1.47 (m, 1H), 1.37 (s, 9H), 1.09-1.02 (m, 1H), 0.45-0.39 (m, 1H).

The following intermediates in Table 103 were prepared using the method described above in Step 4 for the preparation of racemic-tert-butyl (6S,7R)-7-cyano-6-(3-methoxyphenyl)-4-azaspiro[2.4]heptane-4-carboxylate utilizing the appropriate starting materials and modifications.

TABLE 103

| Name | Structure | LCMS | NMR |
|---|---|---|---|
| Racemic-tert-butyl (6R,7S)- 7-cyano-6-(3-fluorophenyl)- 4-azaspiro[2.4]heptane-4- carboxylate | 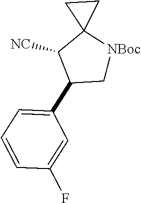 [SMILES: CC(C)(C)OC(=O)N1C[C@@H](c2cccc(F)c2)[C@H](C#N)C12CC2] | | |
| Racemic-tert-butyl (6R,7S)- 7-cyano-6-(3- (difluoromethoxy)phenyl)-4- azaspiro[2.4]heptane-4- carboxylate | 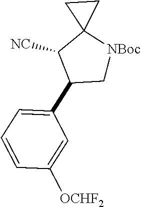 [SMILES: CC(C)(C)OC(=O)N1C[C@@H](c2cccc(OC(F)F)c2)[C@H](C#N)C12CC2] | | ¹H NMR (400 MHz, C₆D₆) δ 6.85- 6.77 (m, 1H), 6.73 - 6.65 (m, 2H), 6.59-6.53 (m, 1H), 5.82 (t, J = 73.6 Hz, 1H), 3.64-3.50 (m, 1H), 3.12-3.03 (m, 1H), 2.92-2.80 (m, 1H), 2.55 (d, J = 10.3 Hz, 1H), 2.32-2.00 (m, 1H), 1.80-1.52 (m, 1H), 1.38 (s, 9H), 1.07-0.98 (m, 1H), 0.41-0.36 (m, 1H). |
| Racemic-tert-butyl (6R,7S)- 7-cyano-6-(3-cyanophenyl)- 4-azaspiro[2.4]heptane-4- carboxylate | 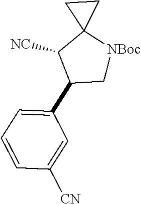 [SMILES: CC(C)(C)OC(=O)N1C[C@@H](c2cccc(C#N)c2)[C@H](C#N)C12CC2] | 268.1 [(M—t- Bu) + 2H]⁺ | ¹H NMR (400 MHz, C₆D₆) δ 6.87 (d, J = 7.6 Hz, 1H), 6.77 (s, 1H), 6.67 (d, J = 8.1 Hz, 1H), 6.58 (t, J = 7.6 Hz, 1H), 3.60-3.38 (m, 1H), 2.96-2.80 (m, 1H), 2.72 (q, J = 9.0 Hz, 1H), 2.40 (d, J = 10.3 Hz, 1H), 2.29-2.03 (m, 1H), 1.76- 1.50 (m, 1H), 1.38 (s, 9H), 1.07- 0.96 (m, 1H), 0.41-0.31 (m, 1H) |
| Racemic-tert-butyl (6R,7S)- 7-cyano-6-(m-tolyl)-4- azaspiro[2.4]heptane-4- carboxylate | 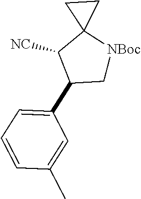 [SMILES: Cc1cccc([C@@H]2CN(C(=O)OC(C)(C)C)C3(CC3)[C@H]2C#N)c1] | 257.2 [(M—t- Bu) + 2H]⁺ | ¹H NMR (400 MHz, C₆D₆) δ 7.06- 6.98 (m, 1H), 6.92-6.86 (m, 1H), 6.76-6.68 (m, 2H), 3.76-3.60 (m, 1H), 3.28-3.15 (m, 1H), 3.11- 2.99 (m, 1H), 2.74 (d, J = 10.8 Hz, 1H), 2.43-2.15 (m, 1H), 2.07 (s, 3H), 1.77-1.57 (m, 1H), 1.38 (s, 9H), 1.14-1.05 (m, 1H), 0.51- 0.41 (m, 1H) |
| Racemic-tert-butyl (6R,7S)- 7-cyano-6-(3- (difluoromethyl)phenyl)-4- azaspiro[2.4]heptane-4- carboxylate | 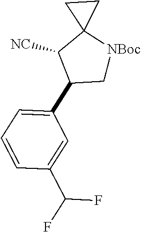 [SMILES: CC(C)(C)OC(=O)N1C[C@@H](c2cccc(C(F)F)c2)[C@H](C#N)C12CC2] | | ¹H NMR (400 MHz, C₆D₆) δ 7.07 (d, J = 7.6 Hz, 1H), 7.02 (s, 1H), 6.90 (t, J = 7.6 Hz, 1H), 6.79 (d, J = 7.6 Hz, 1H), 6.10 (t, J = 56.2 Hz, 1H), 3.65-3.48 (m, 1H), 3.14 - 3.02 (m, 1H), 2.90 (td, J = 10.3, 8.1 Hz, 1H), 2.53 (d, J = 10.3 Hz, 1H), 1.38 (s, 9H), 1.08-0.98 (m, 1H), 0.49 - 0.36 (m, 3H) |

### Step 5: Preparation of Racemic-(6S,7R)-6-(3-methoxyphenyl)-4-azaspiro[2.4]heptane-7-carbonitrile

To a solution of a tert-butyl (6R,7S)-7-cyano-6-(3-methoxyphenyl)-4-azaspiro[2.4]heptane-4-carboxylate (44 mg, 0.1339 mmol, 1 eq) in methylene chloride (2 mL) was added TFA (0.5 mL). The reaction was stirred at room temperature for 2 h. The reaction was concentrated under reduced pressure to give crude racemic-(6S,7R)-6-(3-methoxyphenyl)-4-azaspiro[2.4]heptane-7-carbonitrile (30.5 mg, 99%, TFA salt) as a thick oil. LCMS (ESI) m/z=229.2 [M+H]⁺.

The following intermediates in Table 104 were prepared using the method described above in Step 5 for the preparation of racemic-(6S,7R)-6-(3-methoxyphenyl)-4-azaspiro[2.4]heptane-7-carbonitrile utilizing the appropriate starting materials and modifications.

TABLE 104

| Name | Structure | LCMS |
|---|---|---|
| Racemic-(6R,7S)-6-(3-fluorophenyl)-4-azaspiro[2.4]heptane-7- carbonitrile (TFA salt) | 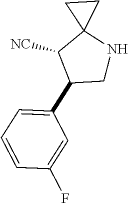 [SMILES: N#C[C@H]1[C@H](c2cccc(F)c2)CNC12CC2] | 217.2 [M + H]⁺ |
| (6R,7S)-6-(3-(difluoromethoxy)phenyl)-4-azaspiro[2.4]heptane- 7-carbonitrile (TFA salt) | 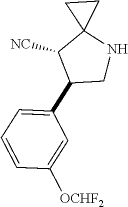 [SMILES: N#C[C@H]1[C@H](c2cccc(OC(F)F)c2)CNC12CC2] | 265.2 [M + H]⁺ |
| (6R,7S)-6-(3-cyanophenyl)-4-azaspiro[2.4]heptane-7- carbonitrile (TFA salt) | 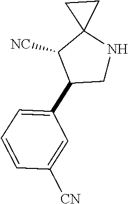 [SMILES: N#Cc1cccc([C@@H]2CNC3(CC3)[C@H]2C#N)c1] | 224.2 [M + H]⁺ |
| (6R,7S)-6-(m-tolyl)-4-azaspiro[2.4]heptane-7-carbonitrile (TFA salt) | 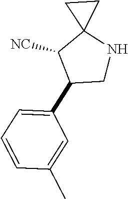 [SMILES: Cc1cccc([C@@H]2CNC3(CC3)[C@H]2C#N)c1] | 213.2 [M + H]⁺ |
| (6R,7S)-6-(3-(difluoromethyl)phenyl)-414-azaspiro[2.4]heptane- 7-carbonitrile (TFA salt) | 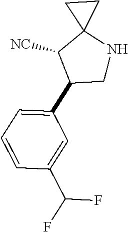 [SMILES: N#C[C@H]1[C@H](c2cccc(C(F)F)c2)CNC12CC2] | 249.2 [M + H]⁺ |

### Step 6: Preparation of Tert-Butyl ((3S,6S,10aS)-3-((6R,7S)-7-cyano-6-(3-methoxyphenyl)-4-azaspiro[2.4]heptane-4-carbonyl)-5-oxodecahydropyrrolo[1,2-a]azocin-6-yl)carbamate and tert-butyl ((3S,6S,10aS)-3-((6S,7R)-7-cyano-6-(3-methoxyphenyl)-4-azaspiro[2.4]heptane-4-carbonyl)-5-oxodecahydropyrrolo[1,2-a]azocin-6-yl)carbamate

To a solution of (6R,7S)-6-(3-methoxyphenyl)-4-azaspiro[2.4]heptane-7-carbonitrile TFA salt (30.5 mg, 0.134 mmol, 1 eq) in DMF (3 mL) were respectively added DIPEA (159 μL, 0.913 mmol, 6.8 eq), (3S,6S,10aS)-6-((tert-butoxycarbonyl)amino)-5-oxodecahydropyrrolo[1,2-a]azocine-3-carboxylic acid (52.1 mg, 0.160 mmol, 1.20 eq) and HATU (63.6 mg, 0.167 mmol, 1.25 eq). The reaction was stirred at room temperature 3 h. The reaction mixture was purified directly by reverse phase chromatography on a 50 g C₁₈ cartridge eluting with 5-60% MeCN in water (with 0.1% formic acid) to afford a 1:1 mixture of two diastereomers (62 mg, 80.4%) as a white solid. LCMS (ESI) m/z=537.2 [M+H]⁺. The mixture of diastereomers was submitted to chiral SFC separation (SFC conditions: Column Lux Cellulose 1, 21.2×150 mm 5 um column, 3.78 mg/inj, concentration 12.3 mg/mL, Column T=40° C., Flow rate 55 mL/min, 10% MeOH, cycle time: 12 min) to give the fastest eluting (Peak 1) diastereomer arbitrarily assigned to tert-butyl ((3S,6S,10aS)-3-((6R,7S)-7-cyano-6-(3-methoxyphenyl)-4-azaspiro[2.4]heptane-4-carbonyl)-5-oxodecahydropyrrolo[1,2-a]azocin-6-yl)carbamate (27 mg, 0.051 mmol, 44% recovery). The slowest eluting (Peak 2) diastereomer was arbitrarily assigned to tert-butyl ((3S,6S,10aS)-3-((6S,7R)-7-cyano-6-(3-methoxyphenyl)-4-azaspiro[2.4]heptane-4-carbonyl)-5-oxodecahydropyrrolo[1,2-a]azocin-6-yl)carbamate (29 mg, 0.054 mmol, 47% recovery).

The following intermediates in Table 105 were prepared using the method described above in Step 6 for the preparation of tert-butyl ((3S,6S,10aS)-3-((6R,7S)-7-cyano-6-(3-methoxyphenyl)-4-azaspiro[2.4]heptane-4-carbonyl)-5-oxodecahydropyrrolo[1,2-a]azocin-6-yl)carbamate and tert-butyl ((3S,6S,10aS)-3-((6S,7R)-7-cyano-6-(3-methoxyphenyl)-4-azaspiro[2.4]heptane-4-carbonyl)-5-oxodecahydropyrrolo[1,2-a]azocin-6-yl)carbamate utilizing the appropriate starting materials and modifications. Compounds were purified by chiral SFC separation.

TABLE 105

| Name | Structure | LCMS |
|---|---|---|
| tert-butyl ((3S,6S,10aS)-3-((6R,7S)-7-cyano- 6-(3-cyanophenyl)-4-azaspiro[2.4]heptane- 4-carbonyl)-5-oxodecahydropyrrolo[1,2- a]azocin-6-yl)carbamate or tert-butyl ((35,6S,10aS)-3-((6S,7R)-7-cyano- 6-(3-cyanophenyl)-4-azaspiro[2.4]heptane- 4-carbonyl)-5-oxodecahydropyrrolo[1,2- a]azocin-6-yl)carbamate Peak 1 (SFC conditions: Column Lux Cellulose 1, 30 × 250 mm 5 um column, 11 mg/inj, concentration 4.4 mg/mL, Column T = 40° C., Flow rate 100 mL/min, 10% EtOH, cycle time: 30 min) | 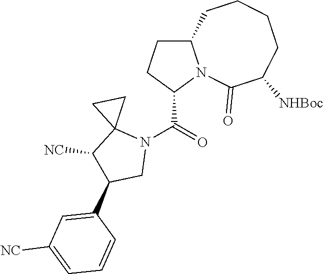 [SMILES: CC(C)(C)OC(=O)N[C@H]1CCCC[C@H]2CC[C@@H](C(=O)N3C[C@@H](c4cccc(C#N)c4)[C@H](C#N)C34CC4)N2C1=O] | 432.1 [(M—Boc) + 2H]⁺ |
| | Or | |
| | 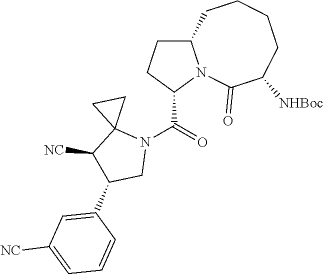 [SMILES: CC(C)(C)OC(=O)N[C@H]1CCCC[C@H]2CC[C@@H](C(=O)N3C[C@H](c4cccc(C#N)c4)[C@@H](C#N)C34CC4)N2C1=O] | |
| tert-butyl ((35,6S,10aS)-3-((65,7R)-7-cyano- 6-(3-cyanophenyl)-4-azaspiro[2.4]heptane- 4-carbonyl)-5-oxodecahydropyrrolo[1,2- a]azocin-6-yl)carbamate or tert-butyl ((35,65,10aS)-3-((6R,7S)-7-cyano- 6-(3-cyanophenyl)-4-azaspiro[2.4]heptane- 4-carbonyl)-5-oxodecahydropyrrolo[1,2- a]azocin-6-yl)carbamate Peak 2 (SFC conditions: Column Lux Cellulose 1, 30 × 250 mm 5 um column, 11 mg/inj, concentration 4.4 mg/mL, Column T = 40° C., Flow rate 100 ml/min, 10% EtOH, cycle time: 30 min) | 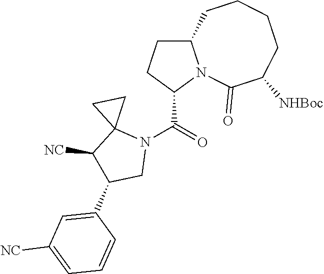 [SMILES: CC(C)(C)OC(=O)N[C@H]1CCCC[C@H]2CC[C@@H](C(=O)N3C[C@H](c4cccc(C#N)c4)[C@@H](C#N)C34CC4)N2C1=O] | 432.2 [(M—Boc) + 2H]⁺ |
| | Or | |
| | 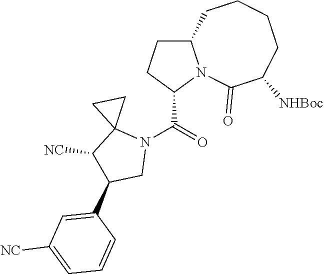 [SMILES: CC(C)(C)OC(=O)N[C@H]1CCCC[C@H]2CC[C@@H](C(=O)N3C[C@@H](c4cccc(C#N)c4)[C@H](C#N)C34CC4)N2C1=O] | |
| tert-butyl ((3S,6S,10aS)-3-((6R,7S)-7-cyano- 6-(m-tolyl)-4-azaspiro[2.4]heptane-4- carbonyl)-5-oxodecahydropyrrolo[1,2- a]azocin-6-yl)carbamate or tert-butyl ((3S,6S,10aS)-3-((6S,7R)-7-cyano- 6-(m-tolyl)-4-azaspiro [2.4]heptane-4- carbonyl)-5-oxodecahydropyrrolo[1,2- a]azocin-6-yl)carbamate Peak 1 (SFC conditions: Column Lux Cellulose 1, 30 × 250 mm 5 um column, 11.3 mg/inj, concentration 22.5 mg/ml, Column T = 40° C., Flow rate 100 mL/min, 10% MeOH, cycle time: 8 min) | 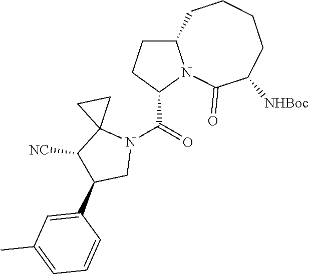 [SMILES: Cc1cccc([C@@H]2CN(C(=O)[C@@H]3CC[C@@H]4CCCC[C@H](NC(=O)OC(C)(C)C)C(=O)N43)C3(CC3)[C@H]2C#N)c1] | 407.4 [(M—Boc) + 2H]⁺ |
| | Or | |
| | 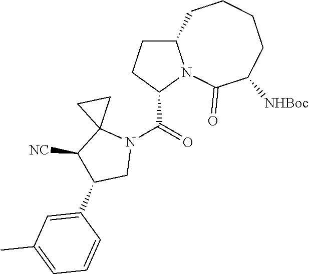 [SMILES: Cc1cccc([C@H]2CN(C(=O)[C@@H]3CC[C@@H]4CCCC[C@H](NC(=O)OC(C)(C)C)C(=O)N43)C3(CC3)[C@@H]2C#N)c1] | |
| tert-butyl ((3S,6S,10aS)-3-((6S,7R)-7-cyano- 6-(m-tolyl)-4-azaspiro[2.4]heptane-4- carbonyl)-5-oxodecahydropyrrolo[1,2- a]azocin-6-yl)carbamate or tert-butyl ((3S,65,10aS)-3-((6R,7S)-7-cyano- 6-(m-tolyl)-4-azaspiro[2.4]heptane-4- carbonyl)-5-oxodecahydropyrrolo[1,2- a]azocin-6-yl)carbamate Peak 2 (SFC conditions: Column Lux Cellulose 1, 30 × 250 mm 5 um column, 11.3 mg/inj, concentration 22.5 mg/ml, Column T = 40° C., Flow rate 100 ml/min, 10% MeOH, cycle time: 8 min) | 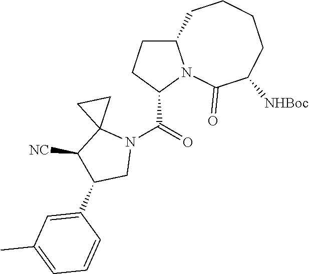 [SMILES: Cc1cccc([C@H]2CN(C(=O)[C@@H]3CC[C@@H]4CCCC[C@H](NC(=O)OC(C)(C)C)C(=O)N43)C3(CC3)[C@@H]2C#N)c1] | 407.4 [(M—Boc) + 2H]⁺ |
| | Or | |
| | 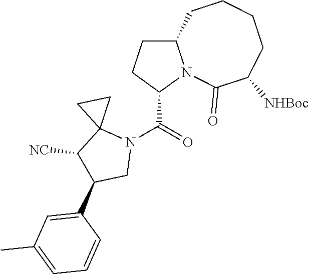 [SMILES: Cc1cccc([C@@H]2CN(C(=O)[C@@H]3CC[C@@H]4CCCC[C@H](NC(=O)OC(C)(C)C)C(=O)N43)C3(CC3)[C@H]2C#N)c1] | |
| tert-butyl ((35,65,10aS)-3-((6R,7S)-7-cyano- 6-(3-(difluoromethyl)phenyl)-4- azaspiro[2.4]heptane-4-carbonyl)-5- oxodecahydropyrrolo[1,2-a]azocin-6- yl)carbamate or tert-butyl ((35,65,10aS)-3-((6S,7R)-7-cyano- 6-(3-(difluoromethyl)phenyl)-4- azaspiro[2.4]heptane-4-carbonyl)-5- oxodecahydropyrrolo[1,2-a]azocin-6- yl)carbamate Peak 1 (SFC conditions: Column Lux Cellulose 1, 30 × 250 mm 5 um column, 4.1 mg/inj, concentration 13.7 mg/ml, Column T = 40° C., Flow rate 75 mL/min, 10% 2-propanol, cycle time: 2.5 min) | 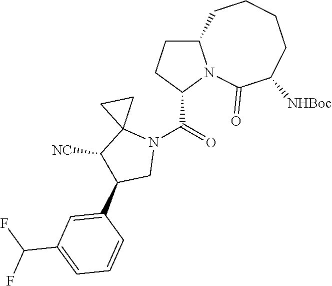 [SMILES: CC(C)(C)OC(=O)N[C@H]1CCCC[C@H]2CC[C@@H](C(=O)N3C[C@@H](c4cccc(C(F)F)c4)[C@H](C#N)C34CC4)N2C1=O] | 557.4 [M + H]⁺ |
| | Or | |
| | 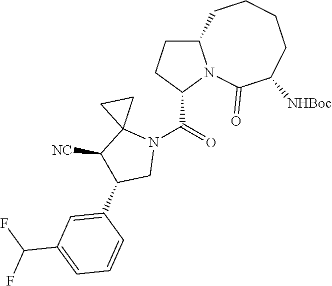 [SMILES: CC(C)(C)OC(=O)N[C@H]1CCCC[C@H]2CC[C@@H](C(=O)N3C[C@H](c4cccc(C(F)F)c4)[C@@H](C#N)C34CC4)N2C1=O] | |
| tert-butyl ((35,65,10aS)-3-((6S,7R)-7-cyano- 6-(3-(difluoromethyl)phenyl)-4- azaspiro[2.4]heptane-4-carbonyl)-5- oxodecahydropyrrolo[1,2-a]azocin-6- yl)carbamate or tert-butyl ((35,6S,10aS)-3-((6R,7S)-7-cyano- 6-(3-(difluoromethyl)phenyl)-4- azaspiro[2.4]heptane-4-carbonyl)-5- oxodecahydropyrrolo[1,2-a]azocin-6- yl)carbamate Peak 2 (SFC conditions: Column Lux Cellulose 1, 30 × 250 mm 5 um column, 4.1 mg/inj, concentration 13.7 mg/mL, Column T = 40° C., Flow rate 75 mL/min, 10% 2-propanol, cycle time: 2.5 min) | 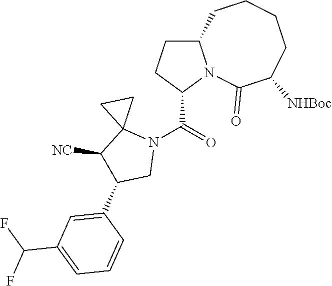 [SMILES: CC(C)(C)OC(=O)N[C@H]1CCCC[C@H]2CC[C@@H](C(=O)N3C[C@H](c4cccc(C(F)F)c4)[C@@H](C#N)C34CC4)N2C1=O] | 557.4 [M + H]⁺ |
| | Or | |
| | 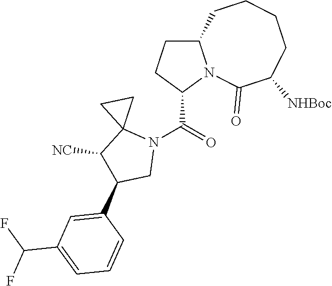 [SMILES: CC(C)(C)OC(=O)N[C@H]1CCCC[C@H]2CC[C@@H](C(=O)N3C[C@@H](c4cccc(C(F)F)c4)[C@H](C#N)C34CC4)N2C1=O] | |

### Synthesis of tert-butyl ((3S,6S,9aS)-3-((6R,7S)-6-(5-chloropyridin-3-yl)-7-cyano-4-azaspiro[2.4]heptane-4-carbonyl)-5-oxooctahydro-1H-pyrrolo[1,2-a]azepin-6-yl)carbamate and tert-butyl ((3S,6S,9aS)-3-((6S,7R)-6-(5-chloropyridin-3-yl)-7-cyano-4-azaspiro[2.4]heptane-4-carbonyl)-5-oxooctahydro-1H-pyrrolo[1,2-a]azepin-6-yl)carbamate

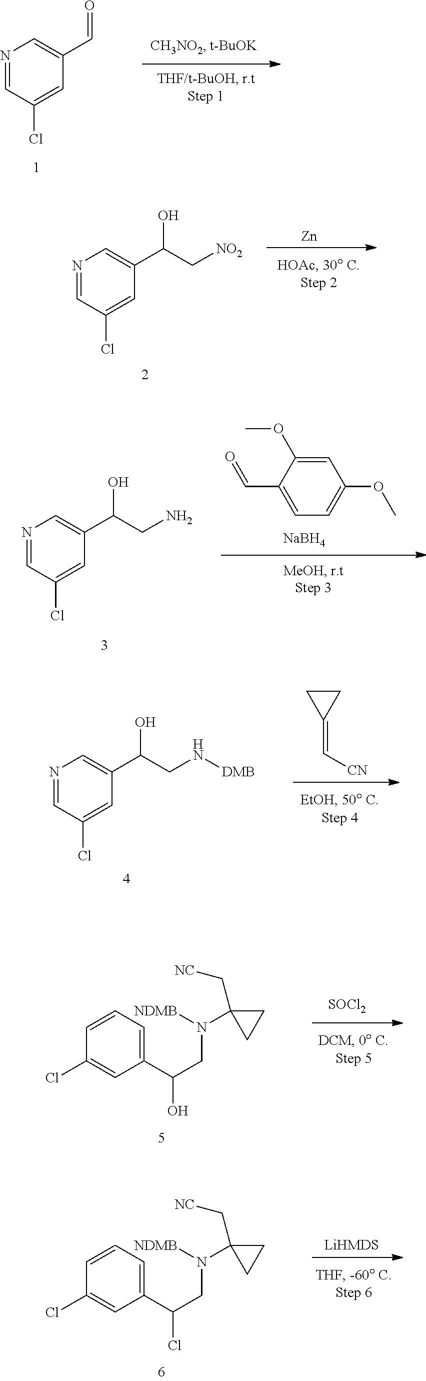

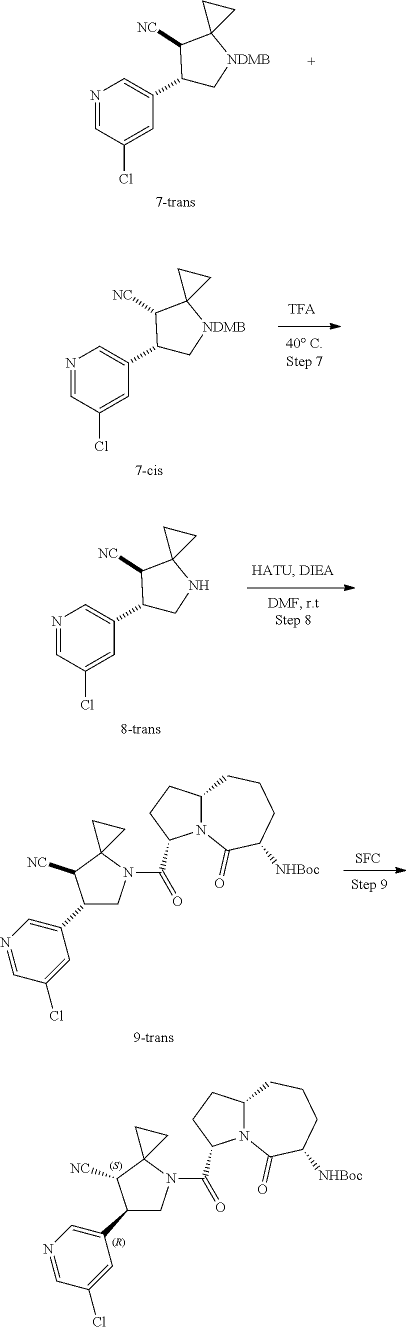

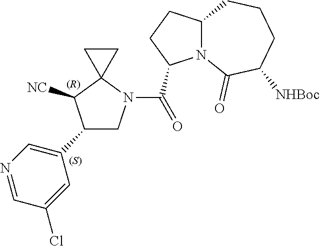

### Step 1: Preparation of 1-(5-chloropyridin-3-yl)-2-nitroethan-1-ol

To a solution of 5-chloropyridine-3-carbaldehyde (1, 1.0 g, 7.06 mmol, 1 eq.) in THF (10 mL) and t-BuOH (10 mL) were added nitromethane (683 mg, 11.2 mmol, 1.6 eq.) and t-BuOK (79 mg, 706 μmol, 0.1 eq.). The resulting mixture was stirred at room temperature for 2 h. After completion, the reaction mixture was quenched by adding NH₄Cl (10 mL, aq., sat.), then extracted with EtOAc (10 mL×3). The organic layers were combined and washed with brine (10 mL), dried over anhydrous Na₂SO₄, and concentrated under reduced pressure. The residue was purified by flash column chromatography on silica gel to afford 1-(5-chloropyridin-3-yl)-2-nitroethan-1-ol (2, 1.1 g, 5.42 mmol, 77%) as a yellow solid. LCMS (ESI) m/z=203 [M+H]⁺. ¹H NMR (400 MHz, DMSO-d₆) δ 8.61 (d, J=14.3 Hz, 2H), 8.01 (s, 1H), 6.38 (s, 1H), 5.39 (s, 1H), 4.97 (d, J=12.8 Hz, 1H), 4.75 (t, J=11.1 Hz, 1H).

### Step 2: Preparation of 2-amino-1-(5-chloropyridin-3-yl)ethan-1-ol

A mixture of 1-(5-chloropyridin-3-yl)-2-nitroethan-1-ol (2, 4.2 g, 20.7 mmol, 1.0 eq.) and Zn (6.69 g, 103 mmol, 5.0 eq.) in AcOH (40 mL) was stirred at 30° C. for 10 h. After completion, the suspension was filtered through a pad of Celite®, the filter cake was washed with MeOH (10 mL). The combined filtrates were concentrated to afford 2-amino-1-(5-chloropyridin-3-yl)ethan-1-ol (3, 3.20 g, quant.) as a yellow oil, which was used directly in next step without further purification. LCMS (ESI) m/z=173 [M+H]⁺.

### Step 3: Preparation of 1-(5-chloropyridin-3-yl)-2-((3,4-dimethylbenzyl)amino)ethan-1-ol

To a solution of 2-amino-1-(5-chloropyridin-3-yl)ethan-1-ol (3, 5.0 g, 28.9 mmol, 1.0 eq.) in MeOH (100 mL) was added 2,4-dimethoxybenzaldehyde (4.8 g, 28.9 mmol, 1.0 eq.). The resulting mixture was stirred at room temperature for 2 h. NaBH₄ (3.63 g, 57.8 mmol, 2.0 eq.) was added and the resulting mixture was stirred at room temperature for 2 h. After completion, the reaction mixture was quenched by adding NH₄Cl (100 mL, aq., sat.), then extracted with EtOAc (100 mL×3). The organic layers were combined and washed with brine (10 mL), dried over anhydrous Na₂SO₄, and concentrated under reduced pressure. The residue was purified by flash column chromatography on silica gel to afford 1-(5-chloropyridin-3-yl)-2-((3,4-dimethylbenzyl)amino)ethan-1-ol (4, 4.20 g, 13.0 mmol, 45%) as a yellow oil. LCMS (ESI) m/z=323 [M+H]⁺.

### Step 4: Preparation of 2-(1-((2-(5-chloropyridin-3-yl)-2-hydroxyethyl)(3,4-dimethylbenzyl)amino)cyclopropyl)acetonitrile

To a solution of 1-(5-chloropyridin-3-yl)-2-((3,4-dimethylbenzyl)amino)ethan-1-ol (4, 4.2 g, 13.0 mmol, 1 eq.) in EtOH (50 mL) was added 2-cyclopropylideneacetonitrile (1.02 g, 13.0 mmol, 1.0 eq.). The resulting mixture was stirred at 50° C. for 10 h. After completion, the reaction mixture was concentrated under reduced pressure. The residue was purified by flash column chromatography on silica gel to afford 2-(1-((2-(5-chloropyridin-3-yl)-2-hydroxyethyl)(3,4-dimethylbenzyl)amino)cyclopropyl)acetonitrile (5, 3.8 g, 9.45 mmol, 73%) as a yellow oil. LCMS (ESI) m/z=402 [M+H]⁺.

### Step 5: Preparation of 2-(1-((2-chloro-2-(5-chloropyridin-3-yl)ethyl)(3,4-dimethylbenzyl)amino)cyclopropyl)acetonitrile

To a solution of 2-(1-((2-(5-chloropyridin-3-yl)-2-hydroxyethyl)(3,4-dimethylbenzyl)amino)cyclopropyl)acetonitrile (5, 3.8 g, 9.45 mmol, 1.0 eq.) in DCM (50 mL) was added SOCl₂ (19 mL, 18.9 mmol, 2.0 eq.) at 0° C. The resulting mixture was stirred at 0° C. for 1 hr. After completion, the reaction mixture was quenched with NaHCO₃(40 mL, aq., sat.), then extracted with DCM (40 mL×3). The organic layers were combined and washed with brine (40 mL), dried over anhydrous Na₂SO₄, and concentrated under reduced pressure. The residue was purified by flash column chromatography on silica gel to afford 2-(1-((2-chloro-2-(5-chloropyridin-3-yl)ethyl)(3,4-dimethylbenzyl)amino)cyclopropyl)acetonitrile (6, 3.20 g, 7.61 mmol, 81%) as a yellow oil. LCMS (ESI) m/z=420 [M+H]⁺.

### Step 6: Preparation of Racemic-trans-6-(5-chloropyridin-3-yl)-4-(3,4-dimethylbenzyl)-4-azaspiro[2.4]heptane-7-carbonitrile

To a solution of 2-(1-((2-chloro-2-(5-chloropyridin-3-yl)ethyl)(3,4-dimethylbenzyl)amino)cyclopropyl)acetonitrile (6, 2.6 g, 6.18 mmol, 1.0 eq.) in THF (40 mL) was added LiHMDS (12 mL, 12.3 mmol, 2.0 eq., 1 M in THF) slowly −60° C. The resulting mixture was stirred at −60° C. for 3 min. After completion, the reaction mixture was quenched with NH₄Cl (20 mL, aq., sat.), then extracted with EtOAc (30 mL×3). The organic layers were combined and washed with brine (20 mL), dried over anhydrous Na₂SO₄, and concentrated under reduced pressure. The residue was purified by flash column chromatography on silica gel to afford racemic-trans-6-(5-chloropyridin-3-yl)-4-(3,4-dimethylbenzyl)-4-azaspiro[2.4]heptane-7-carbonitrile (7-trans, 1.35 g, 3.51 mmol, 57%) as a yellow oil. LCMS (ESI) m/z=384 [M+H]⁺.

### Step 7: Preparation of Racemic-trans-6-(5-chloropyridin-3-yl)-4-azaspiro[2.4]heptane-7-carbonitrile

A solution of racemic-trans-6-(5-chloropyridin-3-yl)-4-(3,4-dimethylbenzyl)-4-azaspiro[2.4]heptane-7-carbonitrile (7-trans, 1.35 g, 3.51 mmol, 1.0 eq.) in TFA (8 mL) was stirred at 40° C. for 5 h. After completion, the reaction mixture was quenched with NaHCO₃(10 mL, aq., sat.) until the pH was adjusted pH=8, then extracted with EtOAc (10 mL×3). The organic layers were combined and washed with brine (10 mL), dried over anhydrous Na₂SO₄, and concentrated under reduced pressure to afford racemic-trans-6-(5-chloropyridin-3-yl)-4-azaspiro[2.4]heptane-7-carbonitrile (8-trans, 700 mg, quant.) as a yellow oil, which was used directly in next step without further purification. LCMS (ESI) m/z=234 [M+H]⁺.

### Step 8: Preparation of trans-tert-butyl ((3S,6S,9aS)-3-(6-(5-chloropyridin-3-yl)-7-cyano-4-azaspiro[2.4]heptane-4-carbonyl)-5-oxooctahydro-1H-pyrrolo[1,2-a]azepin-6-yl)carbamate

To a solution of trans-6-(5-chloropyridin-3-yl)-4-azaspiro[2.4]heptane-7-carbonitrile (8-trans, 200 mg, 855 μmol, 1.0 eq.) in DMF (5 mL) were added (3S,6S,9aS)-6-((tert-butoxycarbonyl)amino)-5-oxooctahydro-1H-pyrrolo[1,2-a]azepine-3-carboxylic acid (267 mg, 855 μmol, 1.0 eq.), HATU (486 mg, 1.28 mmol, 1.5 eq.) and DIEA (330 mg, 2.56 mmol, 3.0 eq.). The resulting mixture was stirred at room temperature for 1 hr. After completion, the mixture was purified by C18 column chromatography to give diastereomeric mixture of trans-tert-butyl ((3S,6S,9aS)-3-(6-(5-chloropyridin-3-yl)-7-cyano-4-azaspiro[2.4]heptane-4-carbonyl)-5-oxooctahydro-1H-pyrrolo[1,2-a]azepin-6-yl)carbamate (9-trans, 250 mg, 473 μmol, 55%) as a yellow oil. LCMS (ESI) m/z=528 [M+H]⁺.

### Step 9: Preparation of Tert-Butyl ((3S,6S,9aS)-3-((6R,7S)-6-(5-chloropyridin-3-yl)-7-cyano-4-azaspiro[2.4]heptane-4-carbonyl)-5-oxooctahydro-1H-pyrrolo[1,2-a]azepin-6-yl)carbamate and tert-butyl ((3S,6S,9aS)-3-((6S,7R)-6-(5-chloropyridin-3-yl)-7-cyano-4-azaspiro[2.4]heptane-4-carbonyl)-5-oxooctahydro-1H-pyrrolo[1,2-a]azepin-6-yl)carbamate

trans-tert-butyl ((3S,6S,9aS)-3-(6-(5-chloropyridin-3-yl)-7-cyano-4-azaspiro[2.4]heptane-4-carbonyl)-5-oxooctahydro-1H-pyrrolo[1,2-a]azepin-6-yl)carbamate (9-trans, 280 mg, 530 μmol, 1 eq.) was purified by Chiral separation. Absolute trans stereochemistry was arbitrarily assigned.

Peak 1: tert-butyl ((3S,6S,9aS)-3-((6R,7S)-6-(5-chloropyridin-3-yl)-7-cyano-4-azaspiro[2.4]heptane-4-carbonyl)-5-oxooctahydro-1H-pyrrolo[1,2-a]azepin-6-yl)carbamate or tert-butyl ((3S,6S,9aS)-3-((6S,7R)-6-(5-chloropyridin-3-yl)-7-cyano-4-azaspiro[2.4]heptane-4-carbonyl)-5-oxooctahydro-1H-pyrrolo[1,2-a]azepin-6-yl)carbamate (110 mg, 208 μmol, 39%, >99% ee, Retention time: 1.683 min) as a yellow oil. LCMS (ESI): m/z=528 [M+H]⁺.

Peak 2: tert-butyl ((3S,6S,9aS)-3-((6S,7R)-6-(5-chloropyridin-3-yl)-7-cyano-4-azaspiro[2.4]heptane-4-carbonyl)-5-oxooctahydro-1H-pyrrolo[1,2-a]azepin-6-yl)carbamate or tert-butyl ((3S,6S,9aS)-3-((6R,7S)-6-(5-chloropyridin-3-yl)-7-cyano-4-azaspiro[2.4]heptane-4-carbonyl)-5-oxooctahydro-1H-pyrrolo[1,2-a]azepin-6-yl)carbamate (110 mg, 208 μmol, 39%, >99% ee, Retention time: 2.797 min) as a yellow oil. LCMS (ESI): m/z=528 [M+H]⁺.

Analytical method: Column: ChiralCel OZ, 100×4.6 mm I.D., 5 m; Mobile phase: A for CO₂ and B for methanol (0.05% DEA); Gradient: 8 min @40% B; Flow rate: 2.5 mL/min; Column temperature: 40° C. SFC method: Instrument: SHIMADZU PREP SOLUTION SFC, Column: ChiralCel OZ, 250×21.2 mm I.D., 5 μm, Mobile phase: A for CO₂ and B for MEOH+0.1% MEA, Gradient:B 40% Flow rate: 40 mL/min Back pressure: 100 bar Column temperature: 35° C., Wavelength: 220 nm, Cycle-time: 5.5 min, Eluted time: 2 h.

### Synthesis of tert-butyl ((3S,6S,9aS)-3-((6R,7S)-7-cyano-6-(5-methoxypyridin-3-yl)-4-azaspiro[2.4]heptane-4-carbonyl)-5-oxooctahydro-1H-pyrrolo[1,2-a]azepin-6-yl)carbamate and tert-butyl ((3S,6S,9aS)-3-((6S,7R)-7-cyano-6-(5-methoxypyridin-3-yl)-4-azaspiro[2.4]heptane-4-carbonyl)-5-oxooctahydro-1H-pyrrolo[1,2-a]azepin-6-yl)carbamate

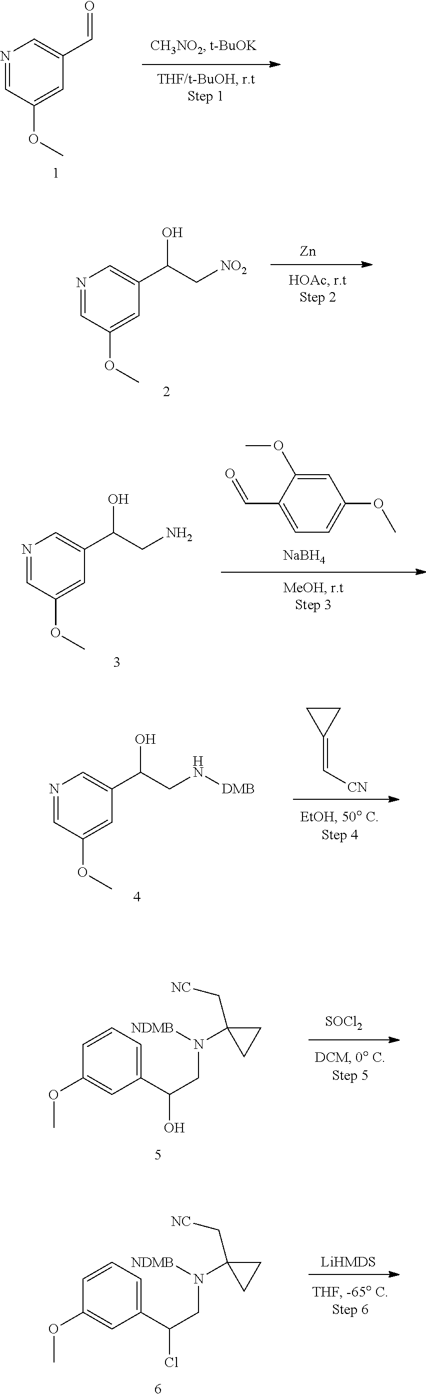

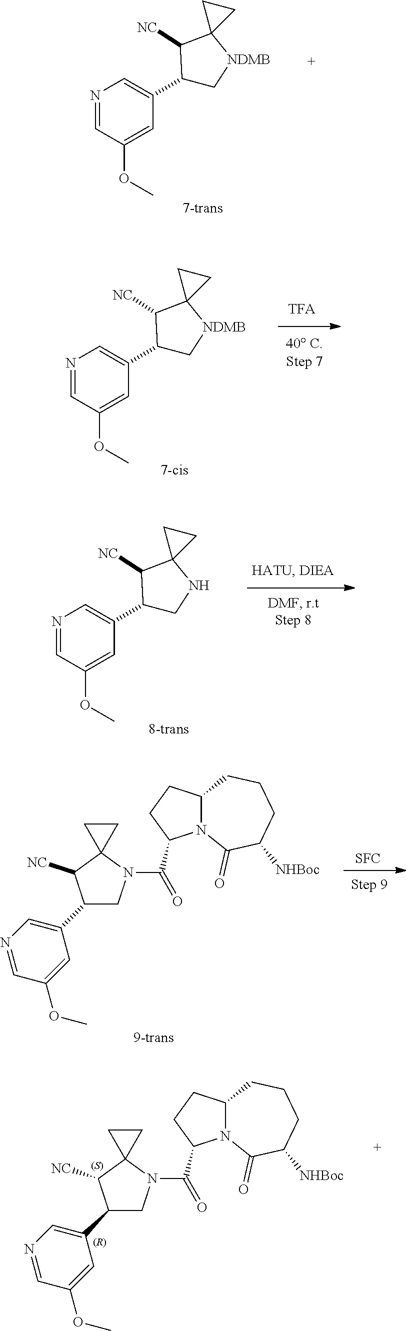

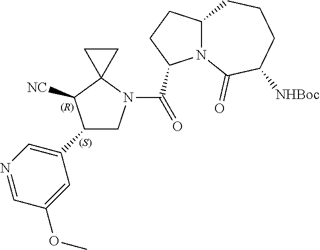

### Step 1: Preparation of 1-(5-methoxypyridin-3-yl)-2-nitroethan-1-ol

To a solution of 5-methoxynicotinaldehyde (1, 3.0 g, 21.8 mmol, 1 eq.) in THF (10 mL) and t-BuOH (10 mL) were added nitromethane (2.12 g, 34.8 mmol, 1.6 eq.) and t-BuOK (244 mg, 2.18 mmol, 0.1 eq.). The resulting mixture was stirred at room temperature for 3 h. After completion, the reaction mixture was quenched by adding NH₄Cl (20 mL, aq., sat.), then extracted with EtOAc (30 mL×3). The organic layers were combined and washed with brine (15 mL), dried over anhydrous Na₂SO₄, and concentrated under reduced pressure. The residue was purified by flash column chromatography on silica gel to afford 1-(5-methoxypyridin-3-yl)-2-nitroethan-1-ol (2, 3.40 g, 17.1 mmol, 79%) as a yellow solid. LCMS (ESI) m/z=199 [M+H]⁺.

### Step 2: Preparation of 2-amino-1-(5-methoxypyridin-3-yl)ethan-1-ol

A mixture of 1-(5-methoxypyridin-3-yl)-2-nitroethan-1-ol (2, 1.0 g, 5.04 mmol, 1.0 eq.) and Zn (1.64 g, 25.2 mmol, 5.0 eq.) in AcOH (10 mL) was stirred at room temperature for 16 h. After completion, the suspension was filtered through a pad of Celite®, the filter cake was washed with MeOH (10 mL). The combined filtrates were concentrated to dryness. The residue was purified by flash column chromatography on silica gel to afford 2-amino-1-(5-methoxypyridin-3-yl)ethan-1-ol (3, 500 mg, 2.97 mmol, 59%) as a yellow oil. LCMS (ESI) m/z=169 [M+H]⁺.

### Step 3: Preparation of 2-((3,4-dimethylbenzyl)amino)-1-(5-methoxypyridin-3-yl)ethan-1-ol

To a solution of 2-amino-1-(5-methoxypyridin-3-yl)ethan-1-ol (3, 800 mg, 4.75 mmol, 1.0 eq.) in MeOH (15 mL) was added 2,4-dimethoxybenzaldehyde (789 mg, 4.75 mmol, 1.0 eq.). The resulting mixture was stirred at room temperature for 2 h. NaBH₄ (596 mg, 9.50 mmol, 2.0 eq.) was added and the resulting mixture was stirred at room temperature for 1 hr. After completion, the reaction mixture was quenched by adding NH₄Cl (10 mL, aq., sat.), then extracted with EtOAc (10 mL×3). The organic layers were combined and washed with brine (10 mL), dried over anhydrous Na₂SO₄, and concentrated under reduced pressure. The residue was purified by flash column chromatography on silica gel to afford 2-((3,4-dimethylbenzyl)amino)-1-(5-methoxypyridin-3-yl)ethan-1-ol (4, 780 mg, 2.44 mmol, 52%) as a yellow oil. LCMS (ESI) m/z=319 [M+H]⁺.

### Step 4: Preparation of 2-(1-((3,4-dimethylbenzyl)(2-hydroxy-2-(5-methoxypyridin-3-yl)ethyl)amino)cyclopropyl)acetonitrile

To a solution of 2-((3,4-dimethylbenzyl)amino)-1-(5-methoxypyridin-3-yl)ethan-1-ol (4, 780 mg, 4.66 mmol, 1 eq.) in EtOH (8 mL) was added 2-cyclopropylideneacetonitrile (368 mg, 4.66 mmol, 1.0 eq.). The resulting mixture was stirred at 50° C. overnight. After completion, the reaction mixture was concentrated under reduced pressure. The residue was purified by flash column chromatography on silica gel to afford 2-(1-((3,4-dimethylbenzyl)(2-hydroxy-2-(5-methoxypyridin-3-yl)ethyl)amino)cyclopropyl)acetonitrile (5, 1 g, 4.04 mmol, 87%) as a yellow oil. LCMS (ESI) m/z=398 [M+H]⁺.

### Step 5: Preparation of 2-(1-((2-chloro-2-(5-methoxypyridin-3-yl)ethyl)(3,4-dimethylbenzyl)amino)cyclopropyl)acetonitrile

To a solution of 2-(1-((3,4-dimethylbenzyl)(2-hydroxy-2-(5-methoxypyridin-3-yl)ethyl)amino)cyclopropyl)acetonitrile (5, 1.46 g, 3.67 mmol, 1.0 eq.) in DCM (10 mL) was added SOCl₂ (873 mg, 7.34 mmol, 2.0 eq.) at 0° C. The resulting mixture was stirred at 0° C. for 1 hr. After completion, the reaction mixture was quenched with NaHCO₃(10 mL, aq., sat.), then extracted with DCM (10 mL×3). The organic layers were combined and washed with brine (10 mL), dried over anhydrous Na₂SO₄, and concentrated under reduced pressure. The residue was purified by flash column chromatography on silica gel to afford 2-(1-((2-chloro-2-(5-methoxypyridin-3-yl)ethyl)(3,4-dimethylbenzyl)amino)cyclopropyl)acetonitrile (6, 1 g, 2.40 mmol, 66%) as a yellow oil. LCMS (ESI) m/z=416 [M+H]⁺.

### Step 6: Preparation of Racemic-trans-4-(3,4-dimethylbenzyl)-6-(5-methoxypyridin-3-yl)-4-azaspiro[2.4]heptane-7-carbonitrile and racemic-cis-4-(3,4-dimethylbenzyl)-6-(5-methoxypyridin-3-yl)-4-azaspiro[2.4]heptane-7-carbonitrile

To a solution of 2-(1-((2-chloro-2-(5-methoxypyridin-3-yl)ethyl)(3,4-dimethylbenzyl)amino)cyclopropyl)acetonitrile (6, 1 g, 2.40 mmol, 1.0 eq.) in THF (30 mL) was added LiHMDS (2.9 mL, 2.88 mmol, 1.2 eq., 1 M in THF) slowly −65° C. The resulting mixture was stirred at −65° C. for 15 min. After completion, the reaction mixture was quenched with NH₄Cl (10 mL, aq., sat.), then extracted with EtOAc (10 mL×3). The organic layers were combined and washed with brine (10 mL), dried over anhydrous Na₂SO₄, and concentrated under reduced pressure. The residue was purified by flash column chromatography on silica gel to afford racemic-trans-4-(3,4-dimethylbenzyl)-6-(5-methoxypyridin-3-yl)-4-azaspiro[2.4]heptane-7-carbonitrile (7-trans, 800 mg, 2.10 mmol, 88%). LCMS (ESI) m/z=380 [M+H]⁺.

### Step 7: Preparation of Racemic-trans-6-(5-methoxypyridin-3-yl)-4-azaspiro[2.4]heptane-7-carbonitrile

A solution of trans-4-(3,4-dimethylbenzyl)-6-(5-methoxypyridin-3-yl)-4-azaspiro[2.4]heptane-7-carbonitrile (7-trans, 880 mg, 2.31 mmol, 1.0 eq.) in TFA (4 mL) was stirred at 40° C. for 4 h. After completion, the reaction mixture was quenched with NaHCO₃ (10 mL, aq., sat.), then extracted with EtOAc (10 mL×3). The organic layers were combined and washed with brine (10 mL), dried over anhydrous Na₂SO₄, and concentrated under reduced pressure to afford racemic-trans-6-(5-methoxypyridin-3-yl)-4-azaspiro[2.4]heptane-7-carbonitrile (8-trans, 570 mg, quant.), which was used directly in next step without further purification. LCMS (ESI) m/z=230 [M+H]⁺.

### Step 8: Preparation of trans-tert-butyl ((3S,6S,9aS)-3-(7-cyano-6-(5-methoxypyridin-3-yl)-4-azaspiro[2.4]heptane-4-carbonyl)-5-oxooctahydro-1H-pyrrolo[1,2-a]azepin-6-yl)carbamate

To a solution of trans-6-(5-methoxypyridin-3-yl)-4-azaspiro[2.4]heptane-7-carbonitrile (8-trans, 130 mg, 566 μmol, 1.0 eq.) in DMF (3 mL) were added (3S,6S,9aS)-6-((tert-butoxycarbonyl)amino)-5-oxooctahydro-1H-pyrrolo[1,2-a]azepine-3-carboxylic acid (176 mg, 566 μmol, 1.0 eq.), HATU (322 mg, 849 μmol, 1.5 eq.) and DIEA (292 mg, 2.26 mmol, 4.0 eq.). The resulting mixture was stirred at room temperature for 1 hr. After completion, the mixture was purified by C18 column chromatography to give trans-tert-butyl ((3S,6S,9aS)-3-(7-cyano-6-(5-methoxypyridin-3-yl)-4-azaspiro[2.4]heptane-4-carbonyl)-5-oxooctahydro-1H-pyrrolo[1,2-a]azepin-6-yl)carbamate (9-trans, 170 mg, 324 μmol, 57%) as a yellow oil. LCMS (ESI) m/z=524 [M+H]⁺.

### Step 9: Preparation of Tert-Butyl ((3S,6S,9aS)-3-((6R,7S)-7-cyano-6-(5-methoxypyridin-3-yl)-4-azaspiro[2.4]heptane-4-carbonyl)-5-oxooctahydro-1H-pyrrolo[1,2-a]azepin-6-yl)carbamate and tert-butyl ((3S,6S,9aS)-3-((6S,7R)-7-cyano-6-(5-methoxypyridin-3-yl)-4-azaspiro[2.4]heptane-4-carbonyl)-5-oxooctahydro-1H-pyrrolo[1,2-a]azepin-6-yl)carbamate

trans-tert-butyl ((3S,6S,9aS)-3-(7-cyano-6-(5-methoxypyridin-3-yl)-4-azaspiro[2.4]heptane-4-carbonyl)-5-oxooctahydro-1H-pyrrolo[1,2-a]azepin-6-yl)carbamate (9-trans, 190 mg, 362 μmol, 1.0 eq.) was purified by Chiral separation. Absolute trans stereochemistry arbitrarily assigned.

Peak 1: tert-butyl ((3S,6S,9aS)-3-((6R,7S)-7-cyano-6-(5-methoxypyridin-3-yl)-4-azaspiro[2.4]heptane-4-carbonyl)-5-oxooctahydro-1H-pyrrolo[1,2-a]azepin-6-yl)carbamate or tert-butyl ((3S,6S,9aS)-3-((6S,7R)-7-cyano-6-(5-methoxypyridin-3-yl)-4-azaspiro[2.4]heptane-4-carbonyl)-5-oxooctahydro-1H-pyrrolo[1,2-a]azepin-6-yl)carbamate (74 mg, 141 μmol, 39%, >99% ee, Retention time: 1.144 min) as a yellow oil. LCMS (ESI): m/z 524.3 [M+H]⁺.

Peak 2: tert-butyl ((3S,6S,9aS)-3-((6S,7R)-7-cyano-6-(5-methoxypyridin-3-yl)-4-azaspiro[2.4]heptane-4-carbonyl)-5-oxooctahydro-1H-pyrrolo[1,2-a]azepin-6-yl)carbamate or tert-butyl ((3S,6S,9aS)-3-((6R,7S)-7-cyano-6-(5-methoxypyridin-3-yl)-4-azaspiro[2.4]heptane-4-carbonyl)-5-oxooctahydro-1H-pyrrolo[1,2-a]azepin-6-yl)carbamate (69 mg, 131 μmol, 37%, >99% ee, Retention time: 1.654 min) as a yellow oil. LCMS (ESI): m/z 524.3 [M+H]⁺.

Analytical method: Instrument: Waters UPC2 analytical SFC (SFC-H) Column: ChiralCel OX, 100×4.6 mm I.D., 3 μm, Mobile phase: A for CO₂ and B for Ethanol (0.05% DEA), Gradient:B 40% Flow rate: 2.5 mL/min, Back pressure: 100 bar, Column temperature: 35° C., Wavelength: 220 nm. SFC method: Instrument: MG II preparative SFC(SFC-13), Column: Cellulose-4, 250×30 mm I.D., 10 μm, Mobile phase: A for CO₂ and B for Ethanol (0.1% NH3H2O), Gradient: B 40% Flow rate: 80 mL/min, Back pressure: 100 bar, Column temperature: 38° C., Wavelength: 220 nm, Cycle time: ˜6 min.

### Synthesis of tert-butyl ((3S,6S,10aS)-3-((6R,7S)-7-cyano-6-(3-chlorophenyl)-4-azaspiro[2.4]heptane-4-carbonyl)-5-oxodecahydropyrrolo[1,2-a]azocin-6-yl)carbamate and tert-butyl ((3S,6S,10aS)-3-((6S,7R)-7-cyano-6-(3-chlorophenyl)-4-azaspiro[2.4]heptane-4-carbonyl)-5-oxodecahydropyrrolo[1,2-a]azocin-6-yl)carbamate

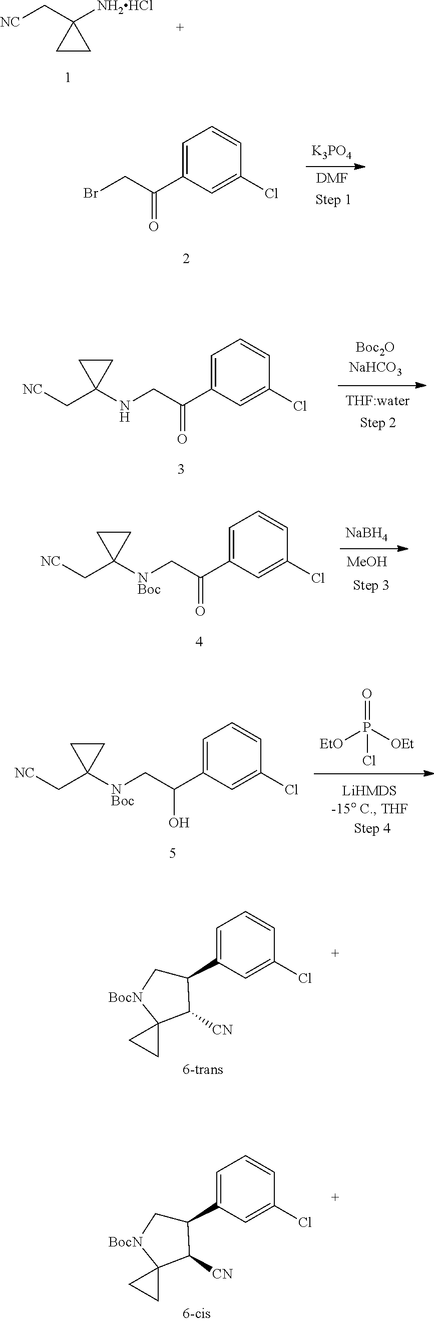

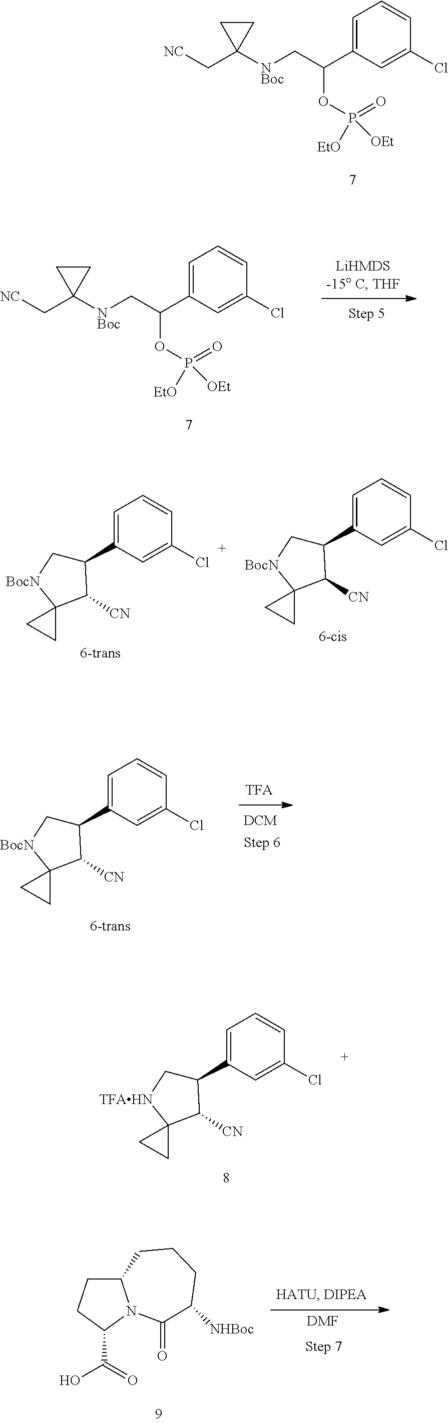

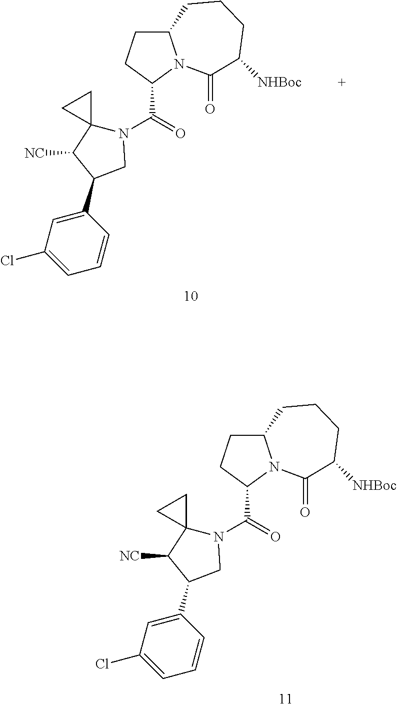 [SMILES: CC(C)(C)OC(=O)N[C@H]1CCC[C@H]2CC[C@@H](C(=O)N3C[C@@H](c4cccc(Cl)c4)[C@H](C#N)C34CC4)N2C1=O ; CC(C)(C)OC(=O)N[C@H]1CCC[C@H]2CC[C@@H](C(=O)N3C[C@H](c4cccc(Cl)c4)[C@@H](C#N)C34CC4)N2C1=O]

### Step 1: Preparation of 2-(1-{[2-(3-Chlorophenyl)-2-oxoethyl]amino}cyclopropyl)acetonitrile

To a solution of 2-(1-aminocyclopropyl)acetonitrile hydrochloride (500 mg, 3.77 mmol, 1 eq) in N,N-dimethylformamide (8 mL) were added 2-bromo-1-(3-chlorophenyl)ethan-1-one (880 mg, 3.77 mmol, 1 eq) followed by potassium phosphate tribasic (1.99 g, 9.42 mmol, 2.5 eq). The mixture was stirred at room temperature for 2 h. An orange mixture was observed over time. The reaction mixture was quenched with the addition of 1 N HCl (25 mL, pH=1) at room temperature. The aqueous solution was washed with EtOAc (2×30 mL) then basified with 1 N NaOH aqueous solution (until pH=9, 25 mL). The product was then extracted with EtOAc (2×60 mL). The combined organic extracts were washed with brine (30 mL), dried with sodium sulfate, filtered and concentrated under reduced pressure to give crude 2-(1-{[2-(3-chlorophenyl)-2-oxoethyl]amino}cyclopropyl)acetonitrile (550 mg, 58.6%) as an orange oil. LCMS (ESI): m/z=249.2 [M+H]⁺.

### Step 2: Preparation of tert-Butyl-N-[2-(3-chlorophenyl)-2-oxoethyl]-N-[1-(cyanomethyl)cyclopropyl]carbamate

To a solution of 2-(1-{[2-(3-chlorophenyl)-2-oxoethyl]amino}cyclopropyl)acetonitrile (550 mg, 2.21 mmol, 1 eq) in TH (4 mL) were added water (4 mL) and sodium bicarbonate (185 mg, 2.21 mmol, 1.0 eq) followed by di-tert-butyl dicarbonate (964 mg, 4.42 mmol, 2 eq). The reaction mixture was stirred for 3 days (over weekend) at room temperature. The reaction mixture was concentrated under reduced pressure (to remove THF). The crude residue was purified by reverse phase chromatography on a 100 g C₁₈ cartridge eluting with 5-80% MeCN in water (with 0.1% formic acid). The combined fractions were concentrated under reduced pressure to give tert-butyl-N-[2-(3-chlorophenyl)-2-oxoethyl]-N-[1-(cyanomethyl)cyclopropyl]carbamate (400 mg, 51.9%) as a yellow oil. LCMS (ESI): m/z=249.2 (M-Boc+2H)+, 293.2 (M−t−Bu+2H)+. ¹H NMR (400 MHz, CDCl₃) δ 7.94-7.90 (m, 1H), 7.82 (t, J=7.8 Hz, 1H), 7.61-7.54 (m, 1H), 7.44 (dt, J=11.7, 7.9 Hz, 1H), 4.70 (d, J=2.0 Hz, 2H), 1.62-1.57 (m, 1H), 1.49-1.45 (m, 1H), 1.38-1.23 (m, 9H), 1.10 (br. s., 2H), 0.97 (br. s., 2H).

### Step 3: Preparation of tert-Butyl N-[2-(3-chlorophenyl)-2-hydroxyethyl]-N-[1-(cyanomethyl)cyclopropyl]carbamate

To a solution of tert-butyl-N-[2-(3-chlorophenyl)-2-oxoethyl]-N-[1-(cyanomethyl)cyclopropyl]carbamate (440 mg, 1.26 mmol, 1 eq) in methanol (10 mL) at 0° C. was added sodium borohydride (47.6 mg, 1.26 mmol, 1.0 eq). The reaction mixture was stirred for 2 h at room temperature. The reaction mixture was quenched with 1 N HCl (1 mL) and concentrated under reduced pressure. The crude residue was purified by reverse phase chromatography on a 50 g C₁₈ cartridge eluting with 5-60% MeCN in water (with 0.1% formic acid). The combined fractions were concentrated under reduced pressure to give tert-butyl-N-[2-(3-chlorophenyl)-2-hydroxyethyl]-N-[1-(cyanomethyl)cyclopropyl]carbamate (115 mg, 26.0%) as a thick yellow oil. LCMS (ESI): m/z=277.2 (M−t−Bu−OH+H)+.

### Step 4: Preparation of tert-Butyl (6R,7S)-6-(3-chlorophenyl)-7-cyano-4-azaspiro[2.4]heptane-4-carboxylate, tert-butyl (6R,7R)-6-(3-chlorophenyl)-7-cyano-4-azaspiro[2.4]heptane-4-carboxylate and tert-butyl-N-[2-(3-chlorophenyl)-2-[(diethoxyphosphoryl)oxy]ethyl]-N-[1-(cyanomethyl)cyclopropyl]carbamate

To a solution of tert-butyl-N-[2-(3-chlorophenyl)-2-hydroxyethyl]-N-[1-(cyanomethyl)cyclopropyl]carbamate (115 mg, 327 μmol, 1 eq) in anhydrous THF (10 mL) at −15° C. were added diethyl phosphorochloridate (49.4 μL, 343 μmol, 1.05 eq) followed by dropwise addition of LiHMDS 1 M in THF (817 μL, 817 μmol, 2.5 eq). The reaction mixture was stirred at −15° C. for 2 h. The reaction mixture was quenched with 1 N HCl (1 mL) and concentrated under reduced pressure. The crude residue was purified by reverse phase chromatography on a 100 g C₁₈ cartridge eluting with 5-70% MeCN in water (with 0.1% formic acid). The combined fractions were concentrated under reduced pressure to give tert-butyl (6R,7S)-6-(3-chlorophenyl)-7-cyano-4-azaspiro[2.4]heptane-4-carboxylate (19.0 mg, 17.5%) as a white solid. LCMS (ESI): m/z=277.2 (M−t−Bu+2H)+. ¹H NMR (400 MHz, CDCl₃) δ 7.36-7.30 (m, 3H), 7.24-7.20 (m, 1H), 4.10-3.98 (m, 1H), 3.66-3.55 (m, 2H), 3.36-3.34 (m, 1H), 2.10-2.00 (m, 1H), 1.50-1.41 (m, 9H), 1.28-1.20 (m, 1H), 1.07-1.01 (m, 1H), 0.73-0.67 (m, 1H). tert-Butyl (6R,7R)-6-(3-chlorophenyl)-7-cyano-4-azaspiro[2.4]heptane-4-carboxylate (6.00 mg, 5.55%) was also isolated as a thick clear oil. LCMS (ESI): m/z=277.2 (M−t−Bu+2H)⁺. tert-Butyl-N-[2-(3-chlorophenyl)-2-[(diethoxyphosphoryl)oxy]ethyl]-N-[1-(cyanomethyl)cyclopropyl]carbamate (75.0 mg, 47.1%) was also isolated as a beige solid. LCMS (ESI): m/z=431.2 (M−t−Bu+2H)+.

### Step 5: Preparation of tert-Butyl (6R,7S)-6-(3-chlorophenyl)-7-cyano-4-azaspiro[2.4]heptane-4-carboxylate

To a solution of tert-butyl-N-[2-(3-chlorophenyl)-2-[(diethoxyphosphoryl)oxy]ethyl]-N-[1-(cyanomethyl)cyclopropyl]carbamate (75 mg, 154 μmol, 1 eq) in THF (5 mL) at −15° C. was added LiHMDS 1 M in THF (231 μL, 231 μmol, 1.5 eq). The reaction mixture was stirred at −15° C. for 2 h. The reaction mixture was quenched with 1 N HCl (1 mL) and concentrated under reduced pressure. The crude residue was purified by reverse phase chromatography on a 50 g C₁₈ cartridge eluting with 5-70% MeCN in water (with 0.1% formic acid). The combined fractions were concentrated under reduced pressure to give tert-butyl (6R,7S)-6-(3-chlorophenyl)-7-cyano-4-azaspiro[2.4]heptane-4-carboxylate (24.0 mg, 46.8%) as a clear oil. LCMS (ESI): m/z=277.2 (M−t−Bu+2H)+, ¹H NMR (400 MHz, CDCl₃) δ 7.36-7.30 (m, 3H), 7.24-7.20 (m, 1H), 4.10-3.98 (m, 1H), 3.66-3.55 (m, 2H), 3.36-3.34 (m, 1H), 2.10-2.00 (m, 1H), 1.50-1.41 (m, 9H), 1.28-1.20 (m, 1H), 1.07-1.01 (m, 1H), 0.73-0.67 (m, 1H).

### Step 6: Preparation of (6R,7S)-6-(3-Chlorophenyl)-4-azaspiro[2.4]heptane-7-carbonitrile TFA salt

To a solution of a tert-butyl (6R,7S)-6-(3-chlorophenyl)-7-cyano-4-azaspiro[2.4]heptane-4-carboxylate (40 mg, 120 μmol, 1 eq) in methylene chloride (2 mL) was added TFA (0.5 mL). The reaction mixture was stirred at room temperature for 2 h. The reaction mixture was concentrated under reduced pressure. The crude TFA salt (41.6 mg, 100%) was used for the next step without further purifications. LCMS (ESI): m/z=233.2 [M+H]⁺.

### Step 7: Preparation of Tert-Butyl ((3S,6S,10aS)-3-((6R,7S)-7-cyano-6-(3-chlorophenyl)-4-azaspiro[2.4]heptane-4-carbonyl)-5-oxodecahydropyrrolo[1,2-a]azocin-6-yl)carbamate and tert-butyl ((3S,6S,10aS)-3-((6S,7R)-7-cyano-6-(3-chlorophenyl)-4-azaspiro[2.4]heptane-4-carbonyl)-5-oxodecahydropyrrolo[1,2-a]azocin-6-yl)carbamate

To a solution of (6R,7S)-6-(3-chlorophenyl)-4-azaspiro[2.4]heptane-7-carbonitrile TFA salt (27 mg, 0.116 mmol, 1 eq) in N,N-dimethylformamide (2 mL) were respectively added DIPEA (121 μL, 0.696 mmol, 6.0 eq), (3S,6S,10aS)-6-((tert-butoxycarbonyl)amino)-5-oxodecahydropyrrolo[1,2-a]azocine-3-carboxylic acid (37.8 mg, 0.116 mmol, 1 eq) and HATU (48.2 mg, 0.127 mmol, 1.1 eq). The reaction was stirred at room temperature 3 h. The reaction mixture was purified directly by reverse phase chromatography on a 50 g C₁₈ cartridge eluting with 5-60% MeCN in water (with 0.1% formic acid) to afford a 1:1 mixture of two diastereomers (40 mg, 63.7%) as a white solid. LCMS (ESI) m/z=541.4 [M+H]⁺. The mixture of diastereomers was submitted to chiral SFC separation (SFC conditions: Column Lux Amylose 1, 21.2×250 mm 5 um column, 8 mg/inj, concentration 10 mg/mL, Column T=40° C., Flow rate 75 mL/min, 10% MeOH, cycle time: 22 min) to give the fastest eluting diastereomer arbitrarily assigned to tert-butyl ((3S,6S,10aS)-3-((6R,7S)-7-cyano-6-(3-chlorophenyl)-4-azaspiro[2.4]heptane-4-carbonyl)-5-oxodecahydropyrrolo[1,2-a]azocin-6-yl)carbamate or tert-butyl ((3S,6S,10aS)-3-((6S,7R)-7-cyano-6-(3-chlorophenyl)-4-azaspiro[2.4]heptane-4-carbonyl)-5-oxodecahydropyrrolo[1,2-a]azocin-6-yl)carbamate (11 mg, 0.020 mmol, 27.5% recovery, Peak 1). LCMS (ESI) m/z=541.4 [M+H]⁺. The slowest eluting diastereomer was arbitrarily assigned to tert-butyl ((3S,6S,10aS)-3-((6S,7R)-7-cyano-6-(3-chlorophenyl)-4-azaspiro[2.4]heptane-4-carbonyl)-5-oxodecahydropyrrolo[1,2-a]azocin-6-yl)carbamate or tert-butyl ((3S,6S,10aS)-3-((6R,7S)-7-cyano-6-(3-chlorophenyl)-4-azaspiro[2.4]heptane-4-carbonyl)-5-oxodecahydropyrrolo[1,2-a]azocin-6-yl)carbamate (17 mg, 0.031 mmol, 42.5% recovery, Peak 2). LCMS (ESI) m/z=541.4 [M+H]⁺.

### Synthesis of tert-butyl ((3S,6S,9aS)-3-((6R,7S)-7-cyano-6-(pyridin-3-yl)-4-azaspiro[2.4]heptane-4-carbonyl)-5-oxooctahydro-1H-pyrrolo[1,2-a]azepin-6-yl)carbamate and tert-butyl ((3S,6S,9aS)-3-((6S,7R)-7-cyano-6-(pyridin-3-yl)-4-azaspiro[2.4]heptane-4-carbonyl)-5-oxooctahydro-1H-pyrrolo[1,2-a]azepin-6-yl)carbamate

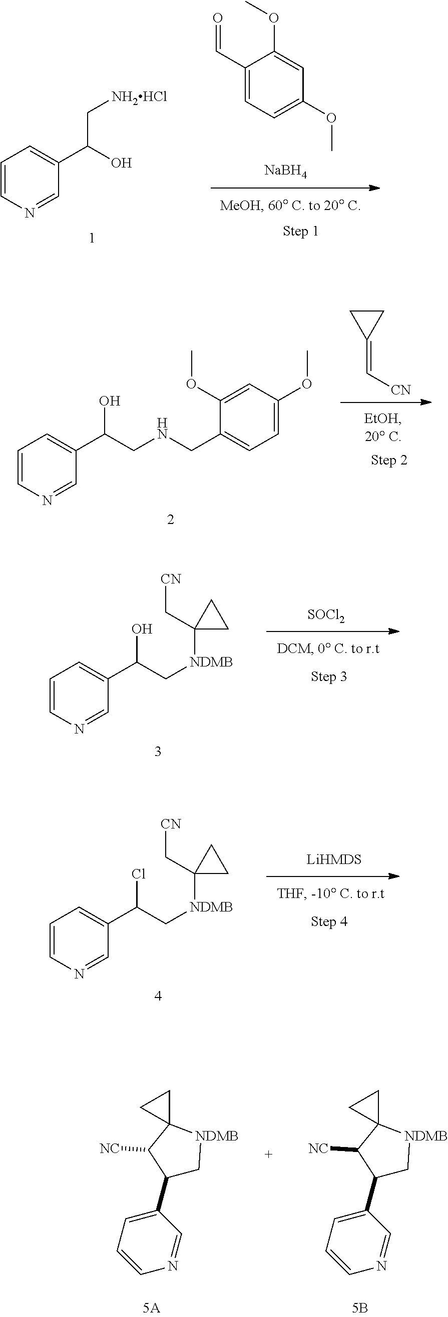

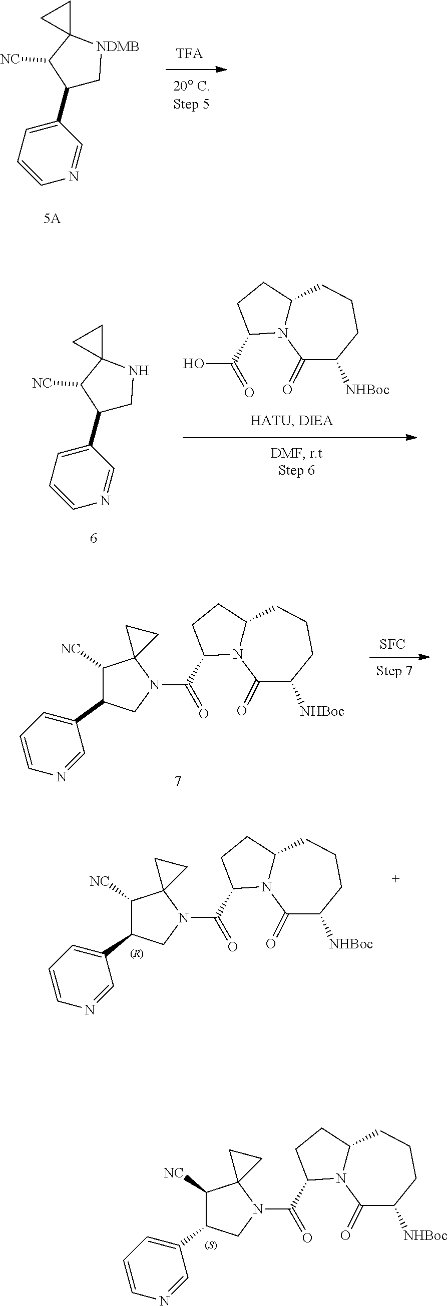

### Step 1: Preparation of 2-((2,4-dimethoxybenzyl)amino)-1-(pyridin-3-yl)ethan-1-ol

A solution of 2-amino-1-(pyridin-3-yl)ethan-1-ol dihydrochloride (1, 2.8 g, 13.2 mmol, 1 eq.) and 2,4-dimethoxybenzaldehyde (2.19 g, 13.2 mmol, 1 eq.) in MeOH (10 mL) was stirred at 60° C. for 0.5 h, then NaBH₄ (1.50 g, 39.5 mmol, 3 eq.) was added at 20° C. and the reaction mixture was stirred at 20° C. for 16 h under N₂ atmosphere. After completion, the reaction mixture was poured into ice-NH₄Cl (100 mL), then extracted with EtOAc (50 mL×3). The organic layers were combined and washed with brine (100 mL), dried over anhydrous Na₂SO₄, and concentrated under reduced pressure to afford 2-((2,4-dimethoxybenzyl)amino)-1-(pyridin-3-yl)ethan-1-ol (2, 2.50 g, 8.67 mmol, 66%) as a yellow oil. LCMS (ESI): m/z=289.2 [M+H]⁺. ¹H NMR (400 MHz, DMSO) δ 8.52 (d, J=2.0 Hz, 1H), 8.44 (dd, J=4.8, 1.6 Hz, 1H), 7.71 (dt, J=7.8, 1.8 Hz, 1H), 7.33 (dd, J=7.7, 4.9 Hz, 1H), 7.13 (d, J=8.2 Hz, 1H), 6.52 (d, J=2.3 Hz, 1H), 6.45 (dd, J=8.2, 2.4 Hz, 1H), 5.46 (s, 1H), 4.70 (t, J=6.0 Hz, 1H), 3.74 (d, J=3.2 Hz, 6H), 3.64 (d, J=4.7 Hz, 2H), 3.17 (d, J=5.1 Hz, 1H), 2.70-2.62 (m, 2H).

### Step 2: Preparation of 2-(1-((3,4-dimethylbenzyl)(2-hydroxy-2-(pyridin-3-yl)ethyl)amino)cyclopropyl)acetonitrile

To a mixture of 2-((2,4-dimethoxybenzyl)amino)-1-(pyridin-3-yl)ethan-1-ol (2, 2.5 g, 8.67 mmol, 1 eq.) in EtOH (5 mL) was added 2-cyclopropylideneacetonitrile in PE solution (1.36 g, 17.3 mmol, 2 eq.) and the resulting mixture was stirred at 20° C. for 1 h under N₂ atmosphere. After completion, the reaction mixture was concentrated under reduced pressure to give a residue. The residue was purified by flash column chromatography on silica gel to get 2-(1-((3,4-dimethylbenzyl)(2-hydroxy-2-(pyridin-3-yl)ethyl)amino)cyclopropyl)acetonitrile (3, 2.40 g, 6.53 mmol, 75%) as a pale-yellow oil. LCMS (ESI): m/z=368.2 [M+H]⁺. ¹H NMR (400 MHz, DMSO) δ 8.40 (dd, J=4.7, 1.6 Hz, 1H), 8.30 (d, J=1.8 Hz, 1H), 7.54 (dt, J=7.8, 1.8 Hz, 1H), 7.28 (dd, J=7.8, 4.8 Hz, 1H), 7.18 (d, J=8.3 Hz, 1H), 6.54 (d, J=2.3 Hz, 1H), 6.47 (dd, J=8.3, 2.4 Hz, 1H), 4.99 (d, J=3.1 Hz, 1H), 4.28 (dd, J=9.8, 6.6 Hz, 1H), 3.85-3.71 (m, 8H), 2.94-2.71 (m, 4H), 0.60-0.31 (m, 3H), 0.06 (s, 1H).

### Step 3: Preparation of 2-(1-((2-chloro-2-(pyridin-3-yl)ethyl)(3,4-dimethylbenzyl)amino)cyclopropyl)acetonitrile

SOCl₂ in DCM solution (3, 281 mg, 2.37 mmol, 1.2 eq.) was added to a solution 2-(1-((3,4-dimethylbenzyl)(2-hydroxy-2-(pyridin-3-yl)ethyl)amino)cyclopropyl)acetonitrile (3, 730 mg, 1.98 mmol, 1 eq.) in DCM (10 mL) at 0° C. The solution was stirred at room temperature for 0.5 h. After completion, the reaction mixture was quenched with NaHCO₃ (aq., sat.) (20 mL), then extracted with EtOAc (50 mL×2). The organic layers were combined and washed with brine (100 mL), dried over anhydrous Na₂SO₄, and concentrated under reduced pressure. The residue was purified by flash column chromatography on silica gel to afford 2-(1-((2-chloro-2-(pyridin-3-yl)ethyl)(3,4-dimethylbenzyl)amino)cyclopropyl)acetonitrile (4, 310 mg, 0.80 mmol, 41%) as an oil. LCMS (ESI): m/z=386.2 [M+H]⁺. ¹H NMR (400 MHz, DMSO) δ 8.50 (dd, J=4.8, 1.5 Hz, 1H), 8.31 (d, J=1.9 Hz, 1H), 7.64-7.57 (m, 1H), 7.37 (dd, J=7.9, 4.8 Hz, 1H), 7.13 (d, J=8.3 Hz, 1H), 6.55 (d, J=2.3 Hz, 1H), 6.49 (dd, J=8.3, 2.4 Hz, 1H), 4.62 (t, J=7.2 Hz, 1H), 3.81-3.72 (m, 8H), 3.36-3.23 (m, 2H), 2.91-2.75 (m, 2H), 0.61-0.38 (m, 3H), 0.06-0.04 (m, 1H).

### Step 4: Preparation of Racemic-trans-4-(3,4-dimethylbenzyl)-6-(pyridin-3-yl)-4-azaspiro[2.4]heptane-7-carbonitrile & racemic-cis-4-(3,4-dimethylbenzyl)-6-(pyridin-3-yl)-4-azaspiro[2.4]heptane-7-carbonitrile

To a solution of 2-(1-((2-chloro-2-(pyridin-3-yl)ethyl)(3,4-dimethylbenzyl)amino)cyclopropyl)acetonitrile (4, 310 mg, 0.80 mmol, 1 eq.) in THF (10 mL) was added LiHMDS (202 mg, 2.00 mmol, 2.5 eq.) dropwise at −10° C. and the solution was stirred at room temperature for 1 hr. After completion, the reaction mixture was quenched with NH₄Cl(aq., sat.) (15 mL), then extracted with EtOAc (15 mL×2). The organic layers were combined and washed with brine (10 mL), dried over anhydrous Na₂SO₄, and concentrated under reduced pressure. The residue was purified by flash column chromatography on silica gel to afford racemic-trans-4-(3,4-dimethylbenzyl)-6-(pyridin-3-yl)-4-azaspiro[2.4]heptane-7-carbonitrile (5A, 75 mg, 0.21 mmol, 27%) and cis-4-(3,4-dimethylbenzyl)-6-(pyridin-3-yl)-4-azaspiro[2.4]heptane-7-carbonitrile (5B, 55.0 mg, 0.16 mmol, 20%) as a yellow oil. LCMS (ESI): m/z=350.2 [M+H]⁺.

### Step 5: Preparation of Racemic-trans-6-(pyridin-3-yl)-4-azaspiro[2.4]heptane-7-carbonitrile

A solution of racemic-trans-4-(3,4-dimethylbenzyl)-6-(pyridin-3-yl)-4-azaspiro[2.4]heptane-7-carbonitrile (5A, 100 mg, 0.29 mmol, 1 eq.) in TFA (5 mL) was stirred at 20° C. overnight under N₂. After completion, the reaction mixture was quenched with NaHCO₃ (aq., sat.) (20 mL), then extracted with EtOAc (20 mL×2). The organic layers were combined and washed with brine (100 mL), dried over anhydrous Na₂SO₄, and concentrated under reduced pressure to afford racemic-trans-6-(pyridin-3-yl)-4-azaspiro[2.4]heptane-7-carbonitrile (6, 55.0 mg, quant.) as an oil for next step without further purification. LCMS (ESI): m/z=199.9 [M+H]⁺.

### Step 6: Preparation of trans-tert-butyl ((3S,6S,9aS)-3-(7-cyano-6-(pyridin-3-yl)-4-azaspiro[2.4]heptane-4-carbonyl)-5-oxooctahydro-1H-pyrrolo[1,2-a]azepin-6-yl)carbamate

To a solution of racemic-trans-6-(pyridin-3-yl)-4-azaspiro[2.4]heptane-7-carbonitrile (6, 900 mg, 4.52 mmol, 1.0 eq.) and DIEA (2.92 g, 22.6 mmol, 5.0 eq.) in DMF (40 mL) were added HATU (1.72 g, 4.52 mol, 1.0 eq.) and (3S,6S,9aS)-6-((tert-butoxycarbonyl)amino)-5-oxooctahydro-1H-pyrrolo[1,2-a]azepine-3-carboxylic acid (1.55 g, 4.97 mol, 1.1 eq.). The solution was stirred at room temperature for 1 hr. After completion, the reaction mixture was diluted with H₂O (50 mL), then extracted with EtOAc (50 mL×2). The organic layers were combined and washed with brine (50 mL), dried over anhydrous Na₂SO₄, and concentrated under reduced pressure. The residue was purified by flash column chromatography on silica gel to afford trans-tert-butyl ((3S,6S,9aS)-3-(7-cyano-6-(pyridin-3-yl)-4-azaspiro[2.4]heptane-4-carbonyl)-5-oxooctahydro-1H-pyrrolo[1,2-a]azepin-6-yl)carbamate (7, 450 mg, 0.91 mmol, 20%) as a white solid. LCMS (ESI): m/z=494 [M+H]⁺.

### Step 7: Preparation of Tert-Butyl ((3S,6S,9aS)-3-((6R,7S)-7-cyano-6-(pyridin-3-yl)-4-azaspiro[2.4]heptane-4-carbonyl)-5-oxooctahydro-1H-pyrrolo[1,2-a]azepin-6-yl)carbamate and tert-butyl ((3S,6S,9aS)-3-((6S,7R)-7-cyano-6-(pyridin-3-yl)-4-azaspiro[2.4]heptane-4-carbonyl)-5-oxooctahydro-1H-pyrrolo[1,2-a]azepin-6-yl)carbamate

trans-tert-butyl ((3S,6S,9aS)-3-(7-cyano-6-(pyridin-3-yl)-4-azaspiro[2.4]heptane-4-carbonyl)-5-oxooctahydro-1H-pyrrolo[1,2-a]azepin-6-yl)carbamate (7, 450 mg, 0.91 mmol) was separated by SFC to give tert-butyl ((3S,6S,9aS)-3-((6R,7S)-7-cyano-6-(pyridin-3-yl)-4-azaspiro[2.4]heptane-4-carbonyl)-5-oxooctahydro-1H-pyrrolo[1,2-a]azepin-6-yl)carbamate or tert-butyl ((3S,6S,9aS)-3-((6S,7R)-7-cyano-6-(pyridin-3-yl)-4-azaspiro[2.4]heptane-4-carbonyl)-5-oxooctahydro-1H-pyrrolo[1,2-a]azepin-6-yl)carbamate (Peak 1, 120 mg, 27%) and tert-butyl ((3S,6S,9aS)-3-((6S,7R)-7-cyano-6-(pyridin-3-yl)-4-azaspiro[2.4]heptane-4-carbonyl)-5-oxooctahydro-1H-pyrrolo[1,2-a]azepin-6-yl)carbamate or tert-butyl ((3S,6S,9aS)-3-((6R,7S)-7-cyano-6-(pyridin-3-yl)-4-azaspiro[2.4]heptane-4-carbonyl)-5-oxooctahydro-1H-pyrrolo[1,2-a]azepin-6-yl)carbamate (Peak 2, 220 mg, 49%) as a white solid. LCMS (ESI): m/z=494 [M+H]⁺. SFC method: Instrument: Waters Thar 80 preparative SFC Column: ChiralCel OX, 250×21.2 mm I.D., 5 μm Mobile phase: A for CO₂ and B for 0.1% 7 mol/L NH₃ in MeOH, Gradient: B 40%, Flow rate: 40 mL/min, Back pressure: 100 bar, Column temperature: 35° C., Wavelength: 254 nm, Cycle-time: 4.2 min, Eluted time: 2H.

### Synthesis of tert-butyl (R)-6-(pyridin-3-yl)-4-azaspiro[2.4]heptane-4-carboxylate and tert-butyl (S)-6-(pyridin-3-yl)-4-azaspiro[2.4]heptane-4-carboxylate

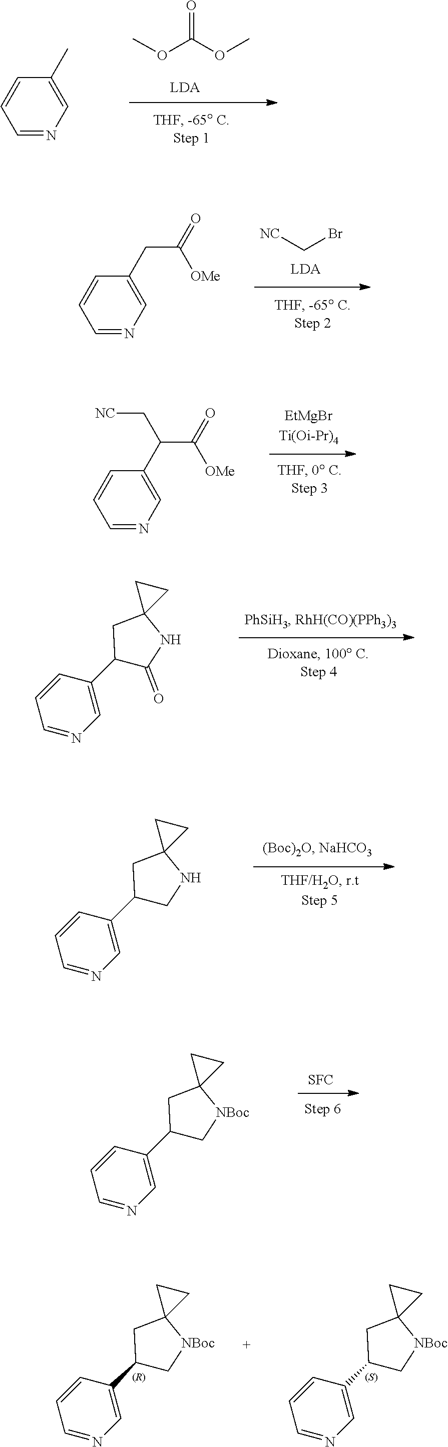

### Step 1: Preparation of Methyl 2-(pyridin-3-yl)acetate

To a solution of 3-methylpyridine (1, 30 g, 322 mmol, 1.0 eq.) in THF (600 mL) was added dropwise LDA (193 mL, 386 mmol, 2M, 1.2 eq.) at −65° C. under N₂ atmosphere. The reaction solution was stirred at −65° C. for 1 h and dimethyl carbonate (57 g, 483 mmol, 1.5 eq.) was added into the reaction mixture. After addition, the mixture was stirred at −65° C. for an additional 1 hr. After completion, the reaction mixture was quenched with saturated NH₄Cl (aq.) solution (500 mL) at 0° C., then extracted with EtOAc (500 mL×2). The organic layers were combined and washed with brine (150 mL), dried over anhydrous Na₂SO₄ and concentrated under reduced pressure. The residue was purified by flash column chromatography on silica gel to afford methyl 2-(pyridin-3-yl)acetate (2, 25.0 g, 165 mmol, 51%) as a yellow oil. LCMS (ESI): m/z=152 [M+H]⁺. ¹H NMR (400 MHz, CDCl₃) δ 8.60-8.48 (m, 2H), 7.68-7.60 (m, 1H), 7.29-7.23 (m, 1H), 3.71 (s, 3H), 3.64 (s, 2H).

### Step 2: Preparation of Methyl 3-cyano-2-(pyridin-3-yl)propanoate

To a solution of LDA (99.0 mL, 198 mmol, 2M, 1.5 eq.) in THF (400 mL) was added dropwise a solution of methyl 2-(pyridin-3-yl)acetate (2, 20 g, 132 mmol, 1.0 eq.) at −65° C. under N₂ atmosphere. The reaction solution was stirred at −65° C. for 1 h and 2-bromoacetonitrile (18.9 g, 158 mmol, 1.2 eq.) was added into the reaction mixture. After addition, the mixture was stirred at −65° C. for an additional 1 h. After completion, the reaction mixture was quenched with saturated ice-NH₄Cl (aq.) solution (500 mL), then extracted with EtOAc (500 mL×3). The organic layers were combined and washed with brine (500 mL), dried over anhydrous Na₂SO₄ and concentrated under reduced pressure. The residue was purified by flash column chromatography on silica gel to afford methyl 3-cyano-2-(2-methoxypyridin-4-yl)propanoate (3, 20.5 g, 107 mmol, 82%) as a yellow oil. LCMS (ESI): m/z=191 [M+H]⁺. ¹H NMR (400 MHz, CDCl₃) δ 8.62 (dd, J=4.8, 1.5 Hz, 1H), 8.57 (d, J=2.2 Hz, 1H), 7.69-7.61 (m, 1H), 7.34 (dd, J=7.9, 4.8 Hz, 1H), 4.00 (t, J=7.5 Hz, 1H), 3.75 (s, 3H), 3.07 (dd, J=16.9, 7.1 Hz, 1H), 2.88 (dd, J=16.9, 7.9 Hz, 1H).

### Step 3: Preparation of 6-(pyridin-3-yl)-4-azaspiro[2.4]heptan-5-one

To a solution of methyl 3-cyano-2-(pyridin-3-yl)propanoate (3, 20.5 g, 107 mmol, 1.0 eq.) and Titanium tetraisopropanolate (36.8 g, 128 mmol, 1.2 eq.) in THF (400 mL) was added dropwise EtMgBr (80 mL, 240 mmol, 3M, 2.25 eq.) at 0° C. After addition, the reaction mixture was stirred at 0° C. for 1 hr. After completion, the reaction mixture was quenched by addition of HCl aqueous solution (140 mL, 2N) at 0° C. The suspension was warmed to room temperature and filtered. The filtrate was poured into ice-water (500 mL) and extracted with EtOAc (200 mL×2). The organic layers were combined, washed with brine (500 mL), dried over anhydrous Na₂SO₄, and concentrated under reduced pressure. The residue was purified by flash column chromatography on silica gel to afford 6-(pyridin-3-yl)-4-azaspiro[2.4]heptan-5-one (4, 9.2 g, 48.8 mmol, 46%) as a yellow solid. LCMS (ESI): m/z=189 [M+H]⁺. ¹H NMR (400 MHz, CDCl₃) δ 8.59 (d, J=2.1 Hz, 1H), 8.53 (dd, J=4.8, 1.5 Hz, 1H), 7.74-7.65 (m, 1H), 7.29 (dd, J=8.0, 4.9 Hz, 1H), 7.00 (s, 1H), 3.91 (t, J=8.7 Hz, 1H), 2.53 (dd, J=12.9, 9.3 Hz, 1H), 2.33 (dd, J=12.9, 8.2 Hz, 1H), 1.00-0.68 (m, 4H).

### Step 4: Preparation of 6-(pyridin-3-yl)-4-azaspiro[2.4]heptane

To a solution of 6-(pyridin-3-yl)-4-azaspiro[2.4]heptan-5-one (4, 3.0 g, 15.9 mmol, 1.0 eq.) and methanidylidyneoxidanium tris(triphenylphosphane) hydrogen rhodium (729 mg, 795 mmol, 0.05 eq.) in dioxane (60 mL) was added phenylsilane (10.32 g, 95.4 mmol, 6.0 eq.). The reaction solution was stirred at 100° C. for 12 h. After completion, the reaction mixture was concentrated under reduced pressure to give a residue. The residue was triturated with 1N HCl (200 mL) at 25° C. for 0.5 h and then filtered. The filtrate was extracted with EtOAc (100 mL×2). The organic layers were combined, dried over anhydrous Na₂SO₄ and concentrated under reduced pressure. The residue was purified by Biotage® C18 column chromatography to afford 6-(pyridin-3-yl)-4-azaspiro[2.4]heptane (5, 2.2 g, 12.62 mmol, 79%) as a brown solid. LCMS (ESI): m/z=175[M+H]⁺. ¹H NMR (400 MHz, CDCl₃) δ 8.55-8.46 (m, 2H), 7.72-7.62 (m, 1H), 7.31-7.25 (m, 1H), 3.84-3.71 (m, 2H), 3.40-3.28 (m, 1H), 2.32-2.17 (m, 2H), 1.52-1.37 (m, 2H), 0.91-0.76 (m, 2H).

### Step 5: Preparation of tert-butyl 6-(pyridin-3-yl)-4-azaspiro[2.4]heptane-4-carboxylate

To a solution of 6-(pyridin-3-yl)-4-azaspiro[2.4]heptane (5, 8 g, 45.9 mmol, 1 eq.) in THF/H₂O (80 mL/10 mL) were added NaHCO₃(7.7 g, 91.7 mmol, 2 eq.) and Boc₂O (12 g, 55.1 mmol, 1.2 eq.). The solution was stirred at room temperature for 4 h. After completion, the reaction mixture was diluted with H₂O (100 mL) and extracted with EtOAc (100 mL×3). The organic layers were combined and washed with brine (100 mL), dried over anhydrous Na₂SO₄, and concentrated under reduced pressure. The residue was purified by flash column chromatography on silica gel to afford tert-butyl 6-(pyridin-3-yl)-4-azaspiro[2.4]heptane-4-carboxylate (6, 6.7 g, 24.5 mmol, 53%) as a white solid. LCMS (ESI): m/z=275 [M+H]⁺.

### Step 6: Preparation of Tert-Butyl (R)-6-(pyridin-3-yl)-4-azaspiro[2.4]heptane-4-carboxylate and tert-butyl (S)-6-(pyridin-3-yl)-4-azaspiro[2.4]heptane-4-carboxylate

tert-butyl 6-(pyridin-3-yl)-4-azaspiro[2.4]heptane-4-carboxylate (6, 6.7 g) was separated by chiral SFC to afford tert-butyl (R)-6-(pyridin-3-yl)-4-azaspiro[2.4]heptane-4-carboxylate (Peak 1, 3 g, 45%) and tert-butyl (S)-6-(pyridin-3-yl)-4-azaspiro[2.4]heptane-4-carboxylate (Peak 2, 3.1 g, 46%). LCMS (ESI): m/z=275 [M+H]⁺. SFC method: Instrument: Waters Thar 80 preparative SFC Column: ChiralPak C-IG, 250×21.2 mm I.D., 5 μm Mobile phase: A for CO₂ and B for MEOH+0.1% NH₃H₂O, Gradient: B 45% Flow rate: 40 mL/min, Back pressure: 100 bar, Column temperature: 35° C., Wavelength: 220 nm Cycle-time: 25 min Eluted time: 7H. The assignment of absolute stereochemical configuration was made by comparison of experimental vibrational circular dichroism (VCD) spectra with theoretical VCD spectra obtained from DFT calculations.

### Synthesis of racemic-(3S,4S)-4-(6-oxo-1,6-dihydropyridin-2-yl)pyrrolidine-3-carbonitrile and racemic-(3S,4S)-4-(6-methoxypyridin-2-yl)pyrrolidine-3-carbonitrile

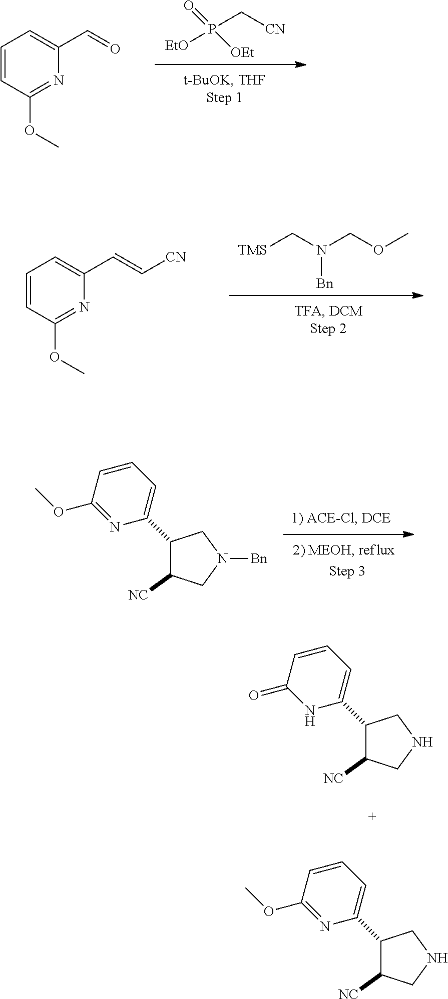

### Step 1: Preparation of (E)-3-(6-methoxypyridin-2-yl)acrylonitrile

To a solution of diethyl (cyanomethyl)phosphonate (1.35 g, 7.65 mmol, 1.05 eq.) in THF (10 mL) was added t-BuOK (897 mg, 8.01 mmol, 1.1 eq.) at 0° C., the solution was stirred for 30 min, then 6-methoxypicolinaldehyde (1 g, 7.29 mmol, 1 eq.) was added to the solution. The solution was stirred at room temperature for 30 min. After completion, H₂O (20 mL) was added to the solution, the solution was extracted with EtOAc (20 mL×3). The organic layers were combined and washed with brine (10 mL), dried over anhydrous Na₂SO₄, and concentrated under reduced pressure. The residue was purified by flash column chromatography on silica gel to afford (E)-3-(6-methoxypyridin-2-yl)acrylonitrile (810 mg, 5.05 mmol, 70%) as a white solid. LCMS (ESI) m/z=161[M+H]⁺. ¹H NMR (400 MHz, DMSO) δ 7.75 (dd, J=8.3, 7.2 Hz, 1H), 7.62 (d, J=16.1 Hz, 1H), 7.15 (d, J=7.1 Hz, 1H), 6.87 (d, J=8.3 Hz, 1H), 6.64 (d, J=16.1 Hz, 1H), 3.86 (s, 3H).

### Step 2: Preparation of Racemic (3S,4S)-1-benzyl-4-(6-methoxypyridin-2-yl)pyrrolidine-3-carbonitrile

To a solution of N-benzyl-1-methoxy-N-((trimethylsilyl)methyl)methanamine (1.19 g, 5.05 mmol, 1 eq.) in DCM (10 mL) were added (E)-3-(6-methoxypyridin-2-yl)acrylonitrile (810 mg, 5.05 mmol, 1 eq.) and TFA (115 mg, 1.01 mmol, 0.2 eq.). The solution was stirred at room temperature overnight. After completion, the reaction mixture was concentrated under reduced pressure. The residue was purified by flash column chromatography on silica gel to afford racemic-(3S,4S)-1-benzyl-4-(6-methoxypyridin-2-yl)pyrrolidine-3-carbonitrile (1.10 g, 3.74 mmol, 74%) as a yellow oil. LCMS (ESI) m/z=294 [M+H]⁺.

### Step 3: Preparation of Racemic-(3S,4S)-4-(6-oxo-1,6-dihydropyridin-2-yl)pyrrolidine-3-carbonitrile and racemic-(3S,4S)-4-(6-methoxypyridin-2-yl)pyrrolidine-3-carbonitrile

To a solution of racemic-(3S,4S)-1-benzyl-4-(6-methoxypyridin-2-yl)pyrrolidine-3-carbonitrile (3, 1.4 g, 4.77 mmol, 1 eq.) in DCE (14 mL) was added 1-chloroethyl carbonochloridate (4.43 g, 31.0 mmol, 6.5 eq.). The solution was stirred at 110° C. for 12 h. After completion, the solution was concentrated under reduced pressure. The residue was dissolved in MeOH (10 mL). The solution was stirred at 60° C. for 30 min. After completion, the reaction mixture was concentrated under reduced pressure to give mixture of racemic-(3S,4S)-4-(6-oxo-1,6-dihydropyridin-2-yl)pyrrolidine-3-carbonitrile and racemic-(3S,4S)-4-(6-methoxypyridin-2-yl)pyrrolidine-3-carbonitrile (900 mg) as a yellow solid. LCMS (ESI) m/z=190 & 204 [M+H]⁺.

### Synthesis of racemic-(3S,4R)-4-(2-oxo-1,2-dihydropyridin-3-yl)pyrrolidine-3-carbonitrile and racemic-(3S,4R)-4-(2-methoxypyridin-3-yl)pyrrolidine-3-carbonitrile

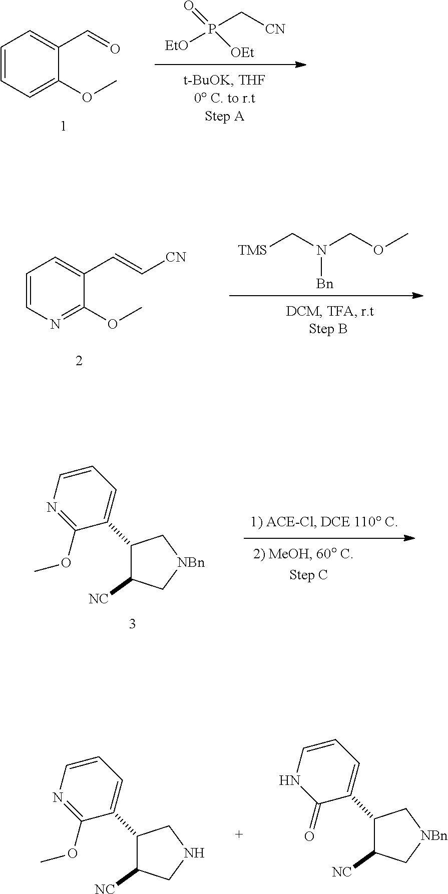

### Step 1: Preparation of (E)-3-(2-methoxypyridin-3-yl)acrylonitrile

To a solution of diethyl (cyanomethyl)phosphonate (2.69 g, 15.2 mmol, 1.05 eq.) in THF (40 mL) was added t-BuOK (1.78 g, 15.9 mmol, 1.1 eq.) at 0° C., the solution was stirred for 30 min, then 2-methoxynicotinaldehyde (1, 2 g, 14.5 mmol, 1 eq.) was added to the solution. The solution was stirred at room temperature for 30 min. After completion, H₂O (40 mL) was added to the solution, the solution was extracted with EtOAc (40 mL×3). The organic layers were combined and washed with brine (10 mL), dried over anhydrous Na₂SO₄, and concentrated under reduced pressure to afford (E)-3-(2-methoxypyridin-3-yl)acrylonitrile (2, 2.10 g, quant.) as a yellow solid, which was used in next step without further purification. LCMS (ESI) m/z=161 [M+H]⁺.

### Step 2: Preparation of Racemic-(3S,4R)-1-benzyl-4-(2-methoxypyridin-3-yl)pyrrolidine-3-carbonitrile

To a solution of (E)-3-(2-methoxypyridin-3-yl)acrylonitrile (2, 2 g, 12.4 mmol, 1 eq.) in DCM (20 mL) were added N-benzyl-1-methoxy-N-((trimethylsilyl)methyl)methanamine (3.22 g, 13.6 mmol, 1.1 eq.) and TFA (282 mg, 2.48 mmol, 0.2 eq.). The reaction solution was stirred at room temperature for 2 h. After completion, the reaction mixture was concentrated under reduced pressure. The residue was purified by Biotage® C18 column chromatography to give racemic-(3S,4R)-1-benzyl-4-(2-methoxypyridin-3-yl)pyrrolidine-3-carbonitrile (3, 2.50 g, 8.52 mmol, 69%) as a yellow oil. LCMS (ESI) m/z=294 [M+H]⁺.

### Step 3: Preparation of Racemic-(3S,4R)-4-(2-oxo-1,2-dihydropyridin-3-yl)pyrrolidine-3-carbonitrile and racemic-(3S,4R)-4-(2-methoxypyridin-3-yl)pyrrolidine-3-carbonitrile

To a solution of racemic-(3S,4R)-1-benzyl-4-(2-methoxypyridin-3-yl)pyrrolidine-3-carbonitrile (3, 2.5 g, 8.52 mmol, 1 eq.) in DCE (25 mL) was added 1-chloroethyl carbonochloridate (7.90 g, 55.3 mmol, 6.5 eq.). The solution was stirred at 110° C. for 12 h. After completion, the solution was concentrated under reduced pressure. The residue was dissolved in MeOH (10 mL). The solution was stirred at 60° C. for 30 min. After completion, the reaction mixture was concentrated under reduced pressure to give a mixture of racemic-(3S,4R)-4-(2-oxo-1,2-dihydropyridin-3-yl)pyrrolidine-3-carbonitrile and racemic-(3S,4R)-4-(2-methoxypyridin-3-yl)pyrrolidine-3-carbonitrile (1.6 g, mixture, quant.) as a yellow solid. LCMS (ESI) m/z=190 & 204 [M+H]⁺.

### Synthesis of (3S,6S,10aS)-6-amino-3-((rel-3R,4R)-3-hydroxy-4-(1H-1,2,3-triazol-1-yl)pyrrolidine-1-carbonyl)octahydropyrrolo[1,2-a]azocin-5(1H)-one

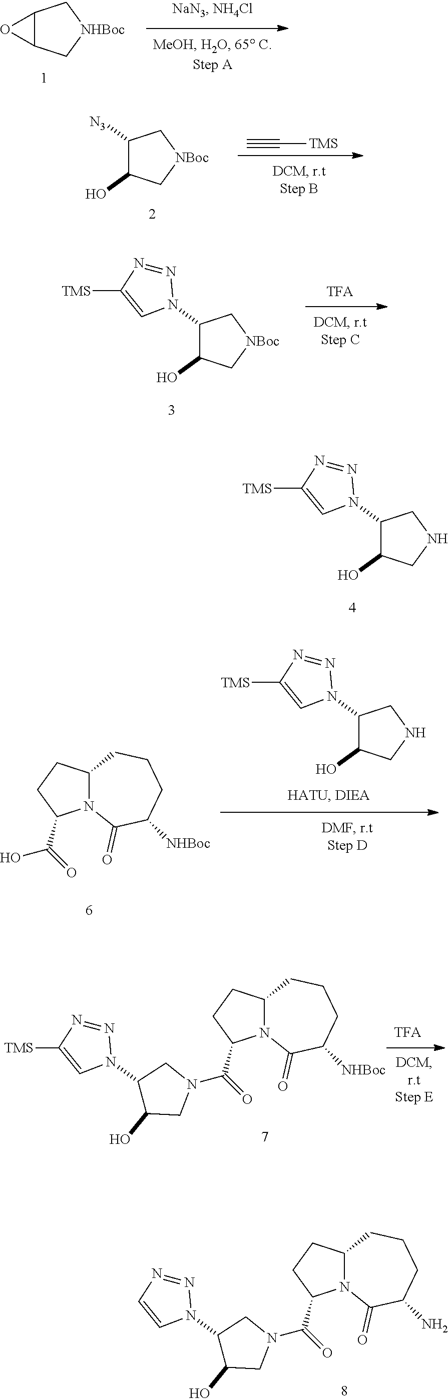

### Step 1: Preparation of Tert-Butyl (rel-3R,4R)-3-azido-4-hydroxypyrrolidine-1-carboxylate

To a solution of tert-butyl 6-oxa-3-azabicyclo[3.1.0]hexane-3-carboxylate (1, 3 g, 16.1 mmol, 1 eq.) and ammonium chloride (3.87 g, 72.4 mmol, 4.5 eq.) in MeOH (96 mL) and H₂O (12 mL) were added NaN₃ (5.23 g, 80.5 mmol, 5 eq.) at room temperature. The resulting mixture was stirred at 60° C. for an additional 18 h. After completion, the reaction mixture was diluted with water and extracted with EtOAc (100 mL×2). The combined organic layers were washed with NaHCO₃ solution, followed by water and then brine, dried over anhydrous Na₂SO₄ and concentrated under reduced pressure to afford tert-butyl (rel-3R,4R)-3-azido-4-hydroxypyrrolidine-1-carboxylate (2, 3.60 g, quant.) as a colorless oil, which was used in next step directly without further purification. LCMS (ESI) m/z=173.2 [M−56+H]⁺.

### Step 2: Preparation of Tert-Butyl (rel-3R,4R)-3-hydroxy-4-(4-(trimethylsilyl)-1H-1,2,3-triazol-1-yl)pyrrolidine-1-carboxylate

To a solution of tert-butyl (rel-3R,4R)-3-azido-4-hydroxypyrrolidine-1-carboxylate (2, 3.6 g, 15.7 mmol, 1 eq.) and ethynyltrimethylsilane (3.85 g, 39.2 mmol, 2.5 eq.) in DCM (40 mL) was added bis(1,5-cyclooctadiene, (Z,Z)—) diiridium dichloride (105 mg, 157 μmol, 0.01 eq.) in portions under N₂. The reaction mixture was stirred at room temperature for 24 h. After completion, the reaction mixture was concentrated under reduced pressure. The residue was purified by flash column chromatography on silica gel to afford tert-butyl (rel-3R,4R)-3-hydroxy-4-(4-(trimethylsilyl)-1H-1,2,3-triazol-1-yl)pyrrolidine-1-carboxylate (3, 2.5 g, 7.67 mmol, 49%) as a brown oil, LCMS (ESI) m/z=327.2 [M+H]⁺.

### Step 3: Preparation of (rel-3R,4R)-4-(4-(trimethylsilyl)-1H-1,2,3-triazol-1-yl)pyrrolidin-3-ol

To a solution of tert-butyl (rel-3R,4R)-3-hydroxy-4-(4-(trimethylsilyl)-1H-1,2,3-triazol-1-yl)pyrrolidine-1-carboxylate (3, 500 mg, 1.53 mmol, 1 eq.) in DCM (20 mL) was added TFA (1.74 g, 15.3 mmol, 10 eq.). The reaction mixture was stirred at room temperature for 1 hr. After completion, the reaction mixture was concentrated under reduced pressure to give (rel-3R,4R)-4-(4-(trimethylsilyl)-1H-1,2,3-triazol-1-yl)pyrrolidin-3-ol (4, 346 mg, quant.) as a brown solid, which was used in next step without further purification.

LCMS (ESI) m/z=227.1 [M+H]⁺.

### Step 4: Preparation of Tert-Butyl ((3S,6S,10aS)-3-((rel-3R,4R)-3-hydroxy-4-(4-(trimethylsilyl)-1H-1,2,3-triazol-1-yl)pyrrolidine-1-carbonyl)-5-oxodecahydropyrrolo[1,2-a]azocin-6-yl)carbamate

To a solution of (3S,6S,10aS)-6-((tert-butoxycarbonyl)amino)-5-oxodecahydropyrrolo[1,2-a]azocine-3-carboxylic acid (6, 100 mg, 0.31 mmol, 1 eq.), DIEA (200 mg, 1.55 mmol, 5 eq.) and (rel-3R,4R)-4-(4-(trimethylsilyl)-1H-1,2,3-triazol-1-yl)pyrrolidin-3-ol (4, 70 mg, 0.31 mmol, 1 eq.) in DMF (1 mL) was added HATU (118 mg, 0.31 mmol, 1.0 eq.) and the reaction mixture was stirred at room temperature overnight under N₂. After completion, the reaction mixture was purified by C18 column chromatography to afford tert-butyl ((3S,6S,10aS)-3-((rel-3R,4R)-3-hydroxy-4-(4-(trimethylsilyl)-1H-1,2,3-triazol-1-yl)pyrrolidine-1-carbonyl)-5-oxodecahydropyrrolo[1,2-a]azocin-6-yl)carbamate (7, 80 mg, 0.16 mmol, 50%). LCMS (ESI): m/z=535 [M+H]⁺.

### Step 5: Preparation of (3S,6S,10aS)-6-amino-3-((rel-3R,4R)-3-hydroxy-4-(1H-1,2,3-triazol-1-yl)pyrrolidine-1-carbonyl)octahydropyrrolo[1,2-a]azocin-5(1H)-one

A solution of tert-butyl ((3S,6S,10aS)-3-((rel-3R,4R)-3-hydroxy-4-(4-(trimethylsilyl)-1H-1,2,3-triazol-1-yl)pyrrolidine-1-carbonyl)-5-oxodecahydropyrrolo[1,2-a]azocin-6-yl)carbamate (7, 80 mg, 0.16 mmol, 1 eq.) in TFA (1 mL) and DCM (3 mL) was stirred at room temperature for 30 min under N₂. After completion, the reaction mixture was concentrated under reduced pressure to afford (3S,6S,10aS)-6-amino-3-((rel-3R,4R)-3-hydroxy-4-(1H-1,2,3-triazol-1-yl)pyrrolidine-1-carbonyl)octahydropyrrolo[1,2-a]azocin-5(1H)-one (8, 80 mg, quant.) as a colorless oil. LCMS (ESI): m/z=363 [M+H]⁺.

### Synthesis of (R)-6-phenyl-4-azaspiro[2.4]heptane and (S)-6-phenyl-4-azaspiro[2.4]heptane

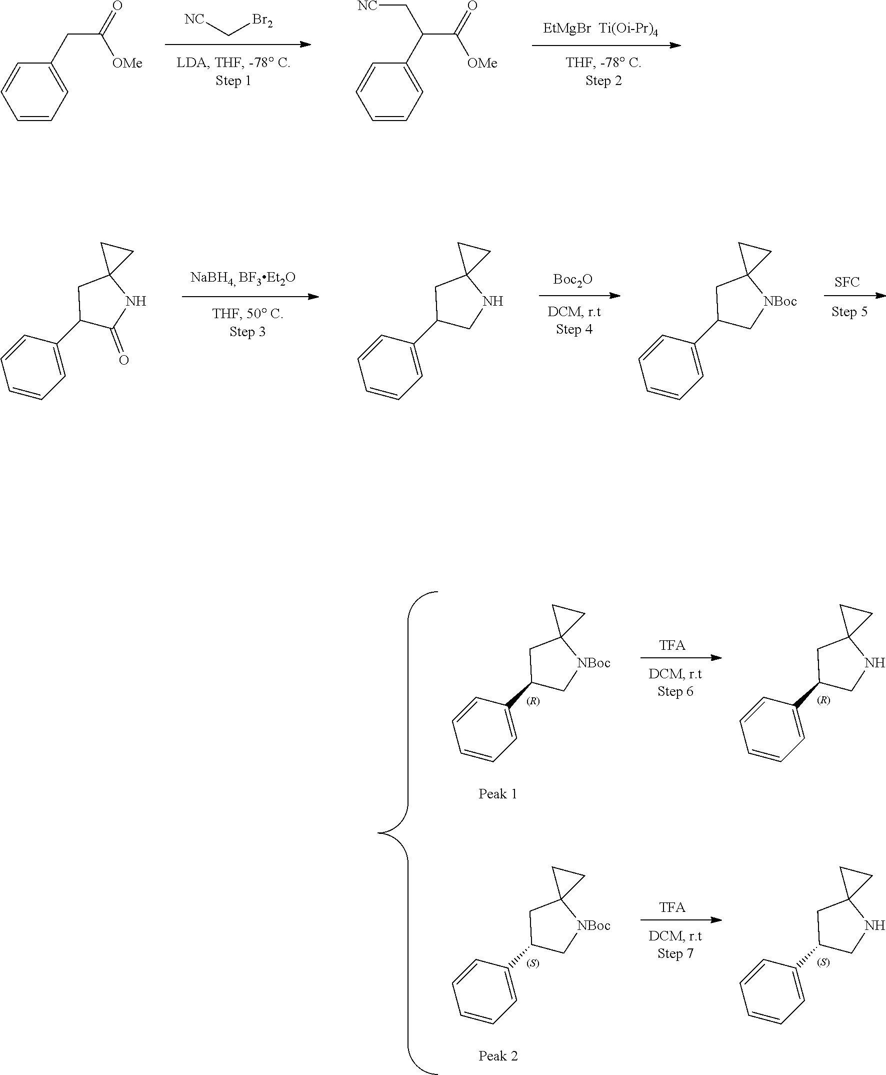

### Step 1: Preparation of Methyl 3-cyano-2-phenylpropanoate

To a solution of LDA (5.95 mL, 47.8 mmol, 1.2 eq) in THF (80 mL) was added dropwise methyl 2-phenylacetate (6 g, 39.9 mmol, 1.0 eq) at −78° C. under N₂ atmosphere. The reaction solution was stirred at −78° C. for 0.5 h and 2-bromoacetonitrile (5.25 g, 43.8 mmol, 1.1 eq) was introduced into the reaction. After addition, the mixture was stirred at −78° C. for 0.5 h. After completion, the reaction mixture was quenched with saturated ice-NH₄Cl (aq.) solution (500 mL), then extracted with EtOAc (500 mL×3). The organic layers were combined and washed with brine (500 mL), dried over anhydrous Na₂SO₄ and concentrated under reduced pressure. The residue was purified by flash column chromatography on silica gel to afford methyl 3-cyano-2-phenylpropanoate (6.20 g, 32.7 mmol, 82%) as a yellow oil. LCMS (ESI): m/z=190 [M+H]⁺.

### Step 2: Preparation of 6-phenyl-4-azaspiro[2.4]heptan-5-one

To a solution of methyl 3-cyano-2-phenylpropanoate (6.2 g, 32.7 mmol, 1 eq) in THF (50 mL) was added dropwise Ti(Oi-Pr)₄ (10.2 g, 35.9 mmol, 1.1 eq) at −78° C. The reaction solution was stirred at −78° C. for 10 min and EtMgBr (72 mL, 71.9 mmol, 2.2 eq) was added dropwise into the reaction mixture. The reaction was stirred at −78° C. for 10 min then warmed up to room temperature for 0.5 h. After completion, the reaction mixture was quenched with H₂O (5 mL), followed by 10% aqueous HCl (50 mL) and EtOAc (500 mL). And 10% aq. NaOH solution was added to the resulting clear mixture until the pH was adjusted pH=8-9. The filtrate was extracted with EtOAc (200 mL×2). The organic layers were combined, washed with brine (500 mL), dried over anhydrous Na₂SO₄ and concentrated under reduced pressure. The residue was purified by flash column chromatography on silica gel to afford 6-phenyl-4-azaspiro[2.4]heptan-5-one (9.2 g, 48.8 mmol, 46%) as a yellow solid. LCMS (ESI): m/z=188.2 [M+H]⁺. ¹H NMR (400 MHz, CDCl₃) δ 7.39-7.24 (m, 6H), 3.86 (dd, J=9.4, 7.6 Hz, 1H), 2.51 (dd, J=12.9, 9.4 Hz, 1H), 2.29 (dd, J=12.9, 7.6 Hz, 1H), 0.85-0.88 (m, 1H), 0.87-0.81 (m, 1H), 0.76-0.64 (m, 2H).

### Step 3: Preparation of 6-phenyl-4-azaspiro[2.4]heptane

To a solution of 6-phenyl-4-azaspiro[2.4]heptan-5-one (2.2 g, 11.7 mmol, 1 eq) in THF (100 mL) was added NaBH₄ (2.21 g, 58.5 mmol, 5 eq) at 0° C., then Boron trifluoride etherate (8.30 g, 58.5 mmol, 5 eq) was added dropwise to the mixture. The mixture was stirred at room temperature for 5 h and then stirred at 50° C. overnight. After completion, the reaction mixture was quenched with NaHCO₃ aqueous solution (20 mL) and extracted with EtOAc(20 mL×2). The organic layers were combined, washed with brine (500 mL), dried over anhydrous Na₂SO₄ and concentrated under reduced pressure. The residue was purified by C18 column chromatography to afford 6-phenyl-4-azaspiro[2.4]heptane (1.50 g, 8.65 mmol, 74%) as an oil. LCMS (ESI): m/z=174.3 [M+H]⁺.

### Step 4: Preparation of tert-butyl 6-phenyl-4-azaspiro[2.4]heptane-4-carboxylate

To a solution of 6-phenyl-4-azaspiro[2.4]heptane (1.2 g, 6.92 mmol, 1 eq), DIEA (1.78 g, 13.8 mmol, 2 eq) and DMAP (84.5 mg, 692 μmol, 0.1 eq) in DCM (20 mL) was added di-tert-butyl dicarbonate (301 mg, 1.38 mmol, 1.2 eq) at room temperature. The reaction solution was stirred at room temperature for 5 h. After completion, the reaction mixture was concentrated under reduced pressure. The residue was purified by flash chromatography on silica gel to get tert-butyl 6-phenyl-4-azaspiro[2.4]heptane-4-carboxylate (1.50 g, 5.48 mmol, 79%) as a white solid. LCMS (ESI): m/z=218.3 [(M-tBu)+H]⁺. ¹H NMR (400 MHz, CDCl₃) δ 7.38-7.19 (m, 5H), 3.98 (s, 1H), 3.54-3.39 (m, 2H), 2.48-2.27 (m, 1H), 1.95 (dd, J=12.2, 5.5 Hz, 1H), 1.52 (s, 2H), 1.43 (s, 9H), 0.55-0.42 (m, 2H)

### Step 5: Preparation of Tert-Butyl (R)-6-phenyl-4-azaspiro[2.4]heptane-4-carboxylate and tert-butyl (S)-6-phenyl-4-azaspiro[2.4]heptane-4-carboxylate

tert-butyl 6-phenyl-4-azaspiro[2.4]heptane-4-carboxylate (1.50 g, 5.48 mmol) was further separated by Chiral SFC to give: SFC Method: Instrument: SHIMADZU PREP SOLUTION SFC, Column: ChiralPak IH, 250×21.2 mm I.D., 5 μm, Mobile phase: A for CO₂ and B for MeOH+0.1% NH₃H₂O, Gradient: B 10%, Flow rate: 10 mL/min, Back pressure: 100 bar, Column temperature: 35° C., Wavelength: 220 nm, Cycle-time: 4 min, Eluted time: 4 h. Absolute stereochemistry was arbitrarily assigned.

Peak 1: tert-butyl (R)-6-phenyl-4-azaspiro[2.4]heptane-4-carboxylate or tert-butyl (S)-6-phenyl-4-azaspiro[2.4]heptane-4-carboxylate (600 mg, 40%); Retention time: 3.287 min, 99% ee. LCMS (ESI): m/z=218.3 [(M-tBu)+H]⁺. ¹H NMR (400 MHz, CDCl₃) δ 7.35-7.21 (m, 5H), 3.99 (s, 1H), 3.54-3.40 (m, 2H), 2.37 (dd, J=13.9, 8.8 Hz, 1H), 1.96 (dd, J=12.1, 5.5 Hz, 1H), 1.43 (s, 9H), 0.57-0.41 (m, 2H).

Peak 2: tert-butyl (S)-6-phenyl-4-azaspiro[2.4]heptane-4-carboxylate or tert-butyl (R)-6-phenyl-4-azaspiro[2.4]heptane-4-carboxylate (600 mg, 40%); Retention time: 3.582 min, 97% ee. LCMS (ESI): m/z=218.3 [(M-tBu)+H]⁺. ¹H NMR (400 MHz, CDCl₃) δ 7.37-7.20 (m, 5H), 3.99 (s, 1H), 3.56-3.38 (m, 2H), 2.37 (dd, J=13.8, 8.8 Hz, 1H), 1.96 (dd, J=12.1, 5.5 Hz, 1H), 1.43 (s, 9H), 0.58-0.42 (m, 2H).

Analytical method: Column: (R,R)-Whelk-01, 250×4.6 mm I.D., 5 um, Mobile phase: A for CO₂ and B for IPA (0.05% DEA), Gradient: 10 min @B 20%, Flow rate: 2.0 mL/min, Back pressure: 100 bar, Column temperature: 35° C.

### Step 6: Preparation of (R)-6-phenyl-4-azaspiro[2.4]heptane or (S)-6-phenyl-4-azaspiro[2.4]heptane

To the solution of tert-butyl (R)-6-phenyl-4-azaspiro[2.4]heptane-4-carboxylate or tert-butyl (S)-6-phenyl-4-azaspiro[2.4]heptane-4-carboxylate (60 mg, 219 μmol, 1 eq, Peak 1) in DCM (2 mL) was added TFA (1 mL) at room temperature. The solution was stirred at room temperature. for 2 h. After completion, the reaction mixture was concentrated under reduced pressure to give (R)-6-phenyl-4-azaspiro[2.4]heptane or (S)-6-phenyl-4-azaspiro[2.4]heptane (70.0 mg, quant.) as an oil, which was used in next step directly without further purification. LCMS (ESI): m/z=174.2 [M+H]⁺.

### Step 7: Preparation of (S)-6-phenyl-4-azaspiro[2.4]heptane or (R)-6-phenyl-4-azaspiro[2.4]heptane

To the solution of tert-butyl (6S)-6-phenyl-4-azaspiro[2.4]heptane-4-carboxylate or tert-butyl (R)-6-phenyl-4-azaspiro[2.4]heptane-4-carboxylate (60 mg, 219 μmol, 1 eq, Peak 2) in DCM (2 mL) was added TFA (1 mL) at room temperature. The solution was stirred at room temperature. for 2 h. After completion, the reaction mixture was concentrated under reduced pressure to give (S)-6-phenyl-4-azaspiro[2.4]heptane or (R)-6-phenyl-4-azaspiro[2.4]heptane (70.0 mg, quant.) as an oil, which was used in next step directly without further purification. LCMS (ESI): m/z=174.2 [M+H]⁺.

### Synthesis of 4-(3-fluoropyrrolidin-3-yl)-1-methylpyridin-2(1H)-one & 4-(3-hydroxypyrrolidin-3-yl)-1-methylpyridin-2(1H)-one

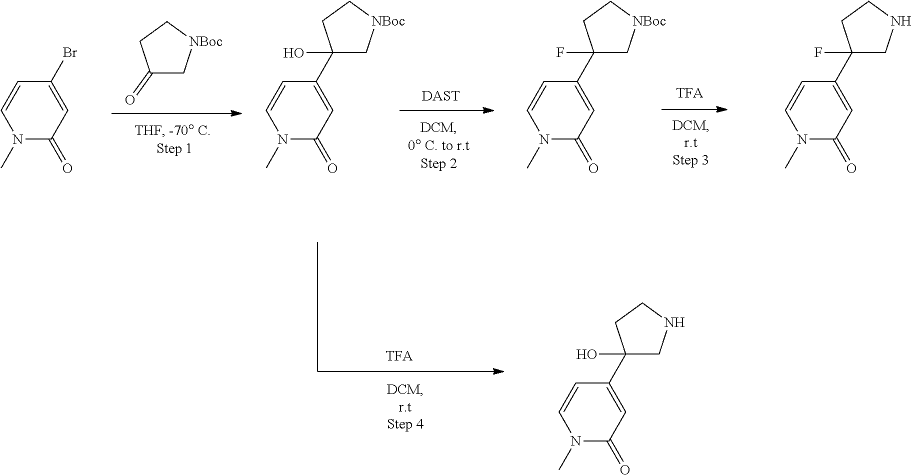

### Step 1: Preparation of Tert-Butyl 3-hydroxy-3-(1-methyl-2-oxo-1,2-dihydropyridin-4-yl)pyrrolidine-1-carboxylate

To a solution of 4-bromo-1-methylpyridin-2(1H)-one (400 mg, 2.12 mmol, 1 eq.) and tert-butyl 3-oxopyrrolidine-1-carboxylate (392 mg, 2.12 mmol, 1 eq.) in THF (6 mL) was added n-BuLi (1.6 M in THF solution) (222 mg, 2.22 mmol, 1.05 eq.) at −70° C. dropwise slowly and stirred at −70° C. for 30 min under N₂. After completion, the reaction mixture was poured into saturate NH₄Cl aqueous solution (20 mL). The mixture was extracted with EtOAc (20 mL×3). The organic phase was washed with brine (50 mL), dried over anhydrous Na₂SO₄ and concentrated under reduced pressure. The residue was purified by flash column chromatography on silica gel to afford tert-butyl 3-hydroxy-3-(1-methyl-2-oxo-1,2-dihydropyridin-4-yl)pyrrolidine-1-carboxylate (460 mg, 1.56 mmol, 74%) as a yellow solid. LCMS (ESI): m/z=295.2 [M+H]⁺. ¹H NMR (400 MHz, DMSO) δ 7.62 (d, J=7.0 Hz, 1H), 6.47 (s, 1H), 6.32 (d, J=7.1 Hz, 1H), 5.52 (s, 1H), 3.51-3.41 (m, 4H), 3.38 (s, 3H), 2.22-2.11 (m, 1H), 1.94-1.86 (m, 1H), 1.42-1.39 (m, 9H).

### Step 2: Preparation of tert-butyl 3-fluoro-3-(1-methyl-2-oxo-1,2-dihydropyridin-4-yl)pyrrolidine-1-carboxylate

To a solution of tert-butyl 3-hydroxy-3-(1-methyl-2-oxo-1,2-dihydropyridin-4-yl)pyrrolidine-1-carboxylate (300 mg, 1.02 mmol, 1 eq.) in DCM (5 mL) was added DAST (165 mg, 1.02 mmol, 1 eq.) at 0° C. The solution was stirred at room temperature for 2 h. After completion, the reaction mixture was quenched with NaHCO₃ (aq.) and extracted with DCM (10 mL×3). The organic phase was washed with brine (10 mL), dried over anhydrous Na₂SO₄ and concentrated under reduced pressure to afford tert-butyl 3-fluoro-3-(1-methyl-2-oxo-1,2-dihydropyridin-4-yl)pyrrolidine-1-carboxylate (300 mg, quant.), which was used in next step directly without further purification. LCMS (ESI): m/z=297 [M+H]⁺.

### Step 3: Preparation of 4-(3-fluoropyrrolidin-3-yl)-1-methylpyridin-2(1H)-one

To a solution of tert-butyl 3-fluoro-3-(1-methyl-2-oxo-1,2-dihydropyridin-4-yl)pyrrolidine-1-carboxylate (300 mg, 1.01 mmol, 1 eq.) in DCM (3 mL) was added TFA (0.6 mL). The solution was stirred at room temperature for 1 h. After completion, the reaction mixture was concentrated under reduced pressure to afford 4-(3-fluoropyrrolidin-3-yl)-1-methylpyridin-2(1H)-one (250 mg, quant., TFA salt) as a white solid. LCMS (ESI) m/z=197 [M+H]⁺.

### Step 4: Preparation of 4-(3-hydroxypyrrolidin-3-yl)-1-methylpyridin-2(1H)-one

To a solution of tert-butyl 3-hydroxy-3-(1-methyl-2-oxo-1,2-dihydropyridin-4-yl)pyrrolidine-1-carboxylate (130 mg, 0.33 mmol, 1 eq.) in DCM (3 mL) was added TFA (0.6 mL). The solution was stirred at room temperature for 1 h. After completion, the reaction mixture was concentrated under reduced pressure to afford 4-(3-hydroxypyrrolidin-3-yl)-1-methylpyridin-2(1H)-one (120 mg, quant., TFA salt) as a white solid. LCMS (ESI) m/z=295 [M+H]⁺.

### Synthesis of 2-methyl-5-(pyrrolidin-3-yl)pyridazin-3(2H)-one

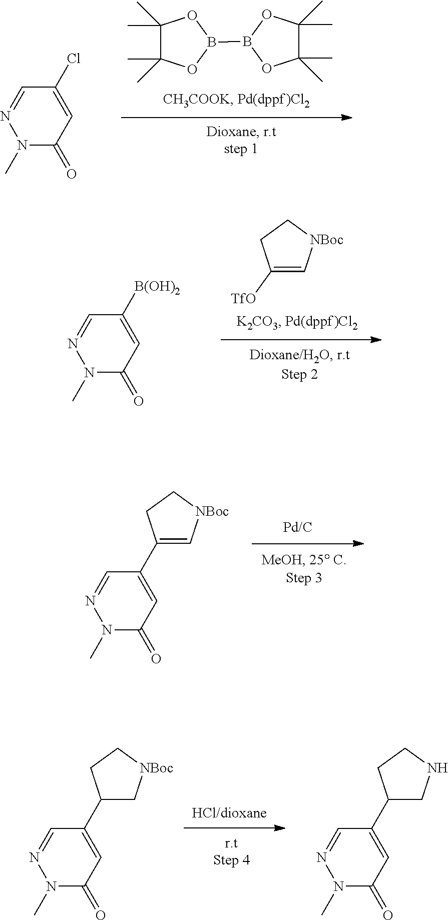

### Step 1: Preparation of (1-methyl-6-oxo-1,6-dihydropyridazin-4-yl)boronic acid

Add 5-chloro-2-methyl-2,3-dihydropyridazin-3-one (500 mg, 3.45 mmol, 1 eq.) to a vial containing 4,4,5,5-tetramethyl-2-(4,4,5,5-tetramethyl-1,3,2-dioxaborolan-2-yl)-1,3,2-dioxaborolane (876 mg, 3.45 mmol, 1.0 eq.), Pd(dppf)Cl₂ (2.52 g, 3.45 mmol, 1.0 eq.), AcOK (1.04 g, 10.3 mmol, 3 eq) in dioxane (3 mL) with an N₂ atmosphere. Allow the mixture to react at room temperature for 24-48 h. Quench the reaction with water. Extract the reaction mixture with EtOAc. Wash the organic layer with brine. Evaporate the organic layer under vacuum. Purify the product by flash chromatography on silica gel to get (1-methyl-6-oxo-1,6-dihydropyridazin-4-yl)boronic acid (400 mg, 2.59 mmol, 75%) as yellow solid. ¹H NMR (400 MHz, DMSO-d6) δ 7.83 (d, J=1.4 Hz, 1H), 7.01 (d, J=1.3 Hz, 1H), 3.64 (s, 3H), 1.30 (s, 12H).

### Step 2: Preparation of tert-butyl 4-(1-methyl-6-oxo-1,6-dihydropyridazin-4-yl)-2,3-dihydro-1H-pyrrole-1-carboxylate

Add (1-methyl-6-oxo-1,6-dihydropyridazin-4-yl)boronic acid (359 mg, 2.33 mmol, 1 eq.) to a vial containing tert-butyl 4-(trifluoromethanesulfonyloxy)-2,3-dihydro-1H-pyrrole-1-carboxylate (885 mg, 2.79 mmol, 1.2 eq.), Pd(dppf)Cl₂ (84.4 mg, 116 μmol, 0.05 eq.), K₂CO₃ (971 mg, 6.98 mmol, 3 eq.) in dioxane (10 mL) and H₂O (2 mL) with N₂ atmosphere. Allow the mixture to react at room temperature for 24-48 h. Quench the reaction with water. Extract the reaction mixture with EtOAc. Wash the organic layer with brine. Evaporate the organic layer under vacuum. Purify the product by flash chromatography on silica gel to get tert-butyl 4-(1-methyl-6-oxo-1,6-dihydropyridazin-4-yl)-2,3-dihydro-1H-pyrrole-1-carboxylate (279 mg, 1.00 mmol, 43%) as a yellow solid. ¹H NMR (400 MHz, DMSO-d6) δ 8.27 (d, J=2.0 Hz, 1H), 6.86 (d, J=11.1 Hz, 1H), 6.70 (d, J=2.0 Hz, 1H), 4.42-4.31 (m, 2H), 4.29-4.21 (m, 2H), 3.63 (s, 3H), 1.44 (d, J=3.8 Hz, 9H).

### Step 3: Preparation of tert-butyl 3-(1-methyl-6-oxo-1,6-dihydropyridazin-4-yl)pyrrolidine-1-carboxylate

To a solution of tert-butyl 4-(1-methyl-6-oxo-1,6-dihydropyridazin-4-yl)-2,3-dihydro-1H-pyrrole-1-carboxylate (285 mg, 1.02 mmol, 1 eq.) in MeOH (3 mL) was added Pd/C (5.42 mg, 51 μmol, 0.05 eq.). The mixture was stirred at 25° C. for 12 h under H₂. After completion, the suspension was filtered through a pad of Celite®, the filter cake was washed with MeOH (20 mL). The combined filtrates were concentrated to dryness to give tert-butyl 3-(1-methyl-6-oxo-1,6-dihydropyridazin-4-yl)pyrrolidine-1-carboxylate (280 mg, quant.) as a white solid, which was used in next step without further purification. LCMS (ESI): m/z=559.2 [M+H]⁺.

### Step 4: Preparation of 2-methyl-5-(pyrrolidin-3-yl)pyridazin-3(2H)-one

A solution of tert-butyl 3-(1-methyl-6-oxo-1,6-dihydropyridazin-4-yl)pyrrolidine-1-carboxylate (285 mg, 1.02 mmol, 1 eq.) in HCl/dioxane (6 mL, 4 M) was stirred at room temperature overnight under N₂. The reaction was concentrated under reduced pressure to afford 2-methyl-5-(pyrrolidin-3-yl)pyridazin-3(2H)-one (180 mg, quant., HCl salt) as a yellow oil. LCMS (ESI): m/z=180.2 [M+H]⁺.

### Synthesis of 1-cyclopropyl-4-(pyrrolidin-3-yl)pyridin-2(1H)-one

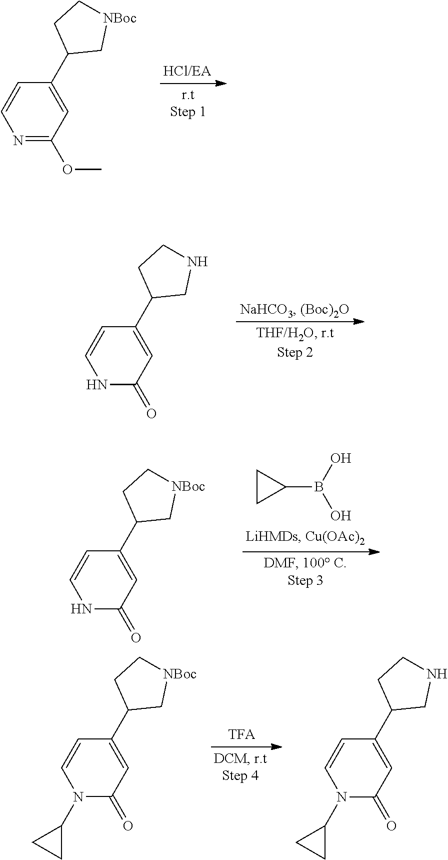

### Step 1: Preparation of 4-(pyrrolidin-3-yl)pyridin-2(1H)-one

A solution of tert-butyl 3-(2-methoxypyridin-4-yl)pyrrolidine-1-carboxylate (400 mg, 1.43 mmol, 1 eq.) in HCl/EA (5 mL) was stirred at room temperature for 2 h. After completion, the reaction mixture was concentrated under reduced pressure to afford 4-(pyrrolidin-3-yl)pyridin-2(1H)-one (230 mg, quant., HCl salt) as a yellow oil. LCMS (ESI): m/z=165 [M+H]⁺.

### Step 2: Preparation of tert-butyl 3-(2-oxo-1,2-dihydropyridin-4-yl)pyrrolidine-1-carboxylate

To a solution of 4-(pyrrolidin-3-yl)pyridin-2(1H)-one (230 mg, 1.40 mmol, 1 eq., HCl salt) in THF/H₂O (5 mL/1 mL) were added NaHCO₃(235 mg, 2.80 mmol, 2 eq.) and Boc₂O (732 mg, 3.36 mmol, 1.2 eq.). The solution was stirred at room temperature for 4 h. After completion, the reaction mixture was diluted with H₂O (10 mL) and extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl 3-(2-oxo-1,2-dihydropyridin-4-yl)pyrrolidine-1-carboxylate (330 mg, 1.38 mmol, 98%) as a white solid. LCMS (ESI): m/z=265 [M+H]⁺.

### Step 3: Preparation of tert-butyl 3-(1-cyclopropyl-2-oxo-1,2-dihydropyridin-4-yl)pyrrolidine-1-carboxylate

A solution of tert-butyl 3-(2-oxo-1,2-dihydropyridin-4-yl)pyrrolidine-1-carboxylate (250 mg, 0.94 mmol, 1.0 eq.), cyclopropylboronic acid (202 mg, 2.35 mmol, 2.5 eq.), Cu(OAc)₂ (257 mg, 1.41 mmol, 1.5 eq.) and LiHMDS (4.23 mmol, 3.0 eq.) in DMF (5 mL), was stirred at 100° C. for additional 2 h by microwave. After completion, the reaction mixture was diluted with H₂O (10 mL), extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl 3-(1-cyclopropyl-2-oxo-1,2-dihydropyridin-4-yl)pyrrolidine-1-carboxylate (33 mg, 108 μmol, 11%) as a white solid. LCMS (ESI): m/z=305 [M+H]⁺.

### Step 4: Preparation of 1-cyclopropyl-4-(pyrrolidin-3-yl)pyridin-2(1H)-one

To a solution of tert-butyl 3-(1-cyclopropyl-2-oxo-1,2-dihydropyridin-4-yl)pyrrolidine-1-carboxylate (360 mg, 1.18 mmol, 1 eq.) in DCM (1 mL) was added TFA (1 mL). The solution was stirred at room temperature for 2 h. After completion, the reaction mixture was concentrated under reduced pressure to afford 1-cyclopropyl-4-(pyrrolidin-3-yl)pyridin-2(1H)-one (217 mg, quant.) as a yellow oil. LCMS (ESI): m/z=205 [M+H]⁺.

### Synthesis of tert-butyl ((3S,6S,10aS)-5-oxo-3-(5-oxo-6-(pyridin-3-yl)-4,6-diazaspiro[2.4]heptane-4-carbonyl)decahydropyrrolo[1,2-a]azocin-6-yl)carbamate

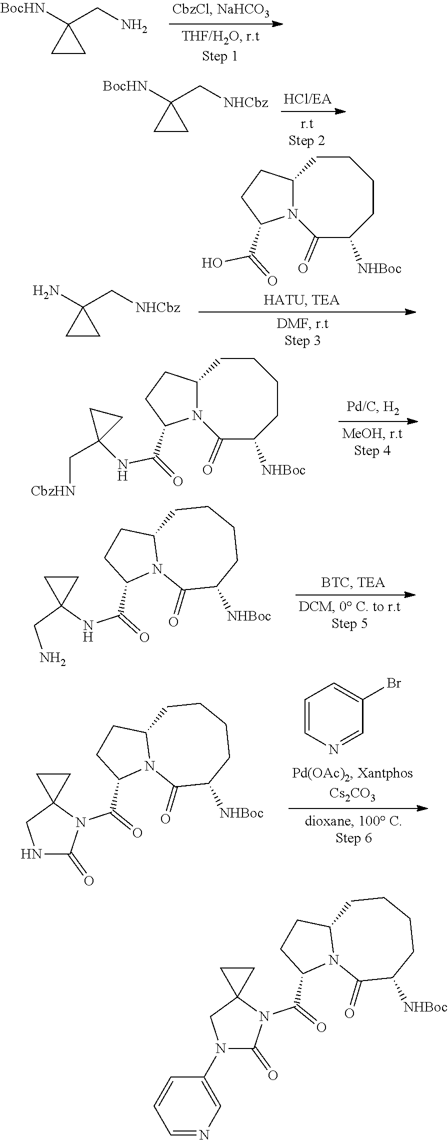

### Step 1: Preparation of Tert-Butyl (1-((((benzyloxy)carbonyl)amino)methyl)cyclopropyl)carbamate

To a solution of tert-butyl (1-(aminomethyl)cyclopropyl)carbamate (400 mg, 2.14 mmol, 1 eq.) in THF/H₂O (5 mL/5 mL) were added NaHCO₃(359 mg, 4.28 mmol, 2 eq.) and benzyl carbonochloridate (436 mg, 2.56 mmol, 1.2 eq.). The solution was stirred at room temperature for 4 h. After completion, the reaction mixture was diluted with H₂O (10 mL), extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl (1-((((benzyloxy)carbonyl)amino)methyl)cyclopropyl)carbamate (600 mg, 1.87 mmol, 88%) as a white solid. LCMS (ESI): m/z=321 [M+H]⁺.

### Step 2: Preparation of Benzyl (1-aminocyclopropyl)methyl)carbamate

To a solution of tert-butyl (1-((((benzyloxy)carbonyl)amino)methyl)cyclopropyl)carbamate (600 mg, 1.87 mmol, 1 eq.) in DCM (5 mL) was added TFA (5 mL). The solution was stirred at room temperature for 2 h. After completion, the reaction mixture was concentrated under reduced pressure to afford benzyl (1-aminocyclopropyl)methyl)carbamate (380 mg, quant.) as a yellow oil. LCMS (ESI): m/z=221 [M+H]⁺.

### Step 3: Preparation of Tert-Butyl ((3S,6S,10aS)-3-((1-((((benzyloxy)carbonyl)amino)methyl)cyclopropyl)carbamoyl)-5-oxodecahydropyrrolo[1,2-a]azocin-6-yl)carbamate

To a solution of (3S,6S,10aS)-6-((tert-butoxycarbonyl)amino)-5-oxodecahydropyrrolo[1,2-a]azocine-3-carboxylic acid (700 mg, 2.14 mmol, 1.0 eq.) in DMF (10 mL) were added HATU (824 mg, 2.17 mmol, 1.02 eq.), TEA (430 mg, 4.26 mmol, 2 eq.) and benzyl (1-aminocyclopropyl)methyl)carbamate (3, 470 mg, 2.13 mmol, 1 eq.). The solution was stirred at room temperature for 1 hr. After completion, the reaction mixture was concentrated under reduced pressure. The residue was purified by Biotage® C18 column chromatography to afford tert-butyl ((3S,6S,10aS)-3-((1-((((benzyloxy)carbonyl)amino)methyl)cyclopropyl)carbamoyl)-5-oxodecahydropyrrolo[1,2-a]azocin-6-yl)carbamate (1.10 g, 2.08 mmol, 98%) as a brown solid. LCMS (ESI): m/z=529 [M+H]⁺.

### Step 4: Preparation of Tert-Butyl ((3S,6S,10aS)-3-((1-(aminomethyl)cyclopropyl)carbamoyl)-5-oxodecahydropyrrolo[1,2-a]azocin-6-yl)carbamate

To a solution of tert-butyl ((3S,6S,10aS)-3-((1-((((benzyloxy)carbonyl)amino)methyl)cyclopropyl)carbamoyl)-5-oxodecahydropyrrolo[1,2-a]azocin-6-yl)carbamate (0.2 g, 2.26 mmol, 1 eq.) in MeOH (10 mL) was added Pd/C (300 mg) under nitrogen. The suspension was degassed under vacuum and purged with H₂ several times. The resulting mixture was stirred at room temperature for 12 h. After completion, the suspension was filtered through a pad of Celite®, the filter cake was washed with MeOH (20 mL). The combined filtrates were concentrated to dryness to give tert-butyl ((3S,6S,10aS)-3-((1-(aminomethyl)cyclopropyl)carbamoyl)-5-oxodecahydropyrrolo[1,2-a]azocin-6-yl)carbamate (850 mg, quant.) as a brown solid. LCMS (ESI): m/z=395 [M+H]⁺.

### Step 5: Preparation of Tert-Butyl ((3S,6S,10aS)-5-oxo-3-(5-oxo-4,6-diazaspiro[2.4]heptane-4-carbonyl)decahydropyrrolo[1,2-a]azocin-6-yl)carbamate

To a solution of tert-butyl ((3S,6S,10aS)-3-((1-(aminomethyl)cyclopropyl)carbamoyl)-5-oxodecahydropyrrolo[1,2-a]azocin-6-yl)carbamate (800 mg, 2.03 mmol, 1 eq.) and TEA (410 mg, 4.06 mmol, 2 eq.) in DCM (10 mL) was added BTC (238 mg, 0.81 mmol, 0.4 eq.). The solution was stirred at room temperature for 1 h. After completion, the reaction mixture was concentrated under reduced pressure. The residue was purified by flash column chromatography on silica gel to afford tert-butyl ((3S,6S,10aS)-5-oxo-3-(5-oxo-4,6-diazaspiro[2.4]heptane-4-carbonyl)decahydropyrrolo[1,2-a]azocin-6-yl)carbamate (120 mg, 0.29 mmol, 14%) as a white solid. LCMS (ESI) m/z=421 [M+H]⁺.

### Step 6: Preparation of Tert-Butyl ((3S,6S,10aS)-5-oxo-3-(5-oxo-6-(pyridin-3-yl)-4,6-diazaspiro[2.4]heptane-4-carbonyl)decahydropyrrolo[1,2-a]azocin-6-yl)carbamate

A solution of tert-butyl ((3S,6S,10aS)-5-oxo-3-(5-oxo-4,6-diazaspiro[2.4]heptane-4-carbonyl)decahydropyrrolo[1,2-a]azocin-6-yl)carbamate (120 mg, 0.29 mmol, 1.0 eq.), 3-bromopyridine (45 mg, 0.29 mmol, 1.0 eq.), and Cs₂CO₃ (284 mg, 0.87 mmol, 2.5 eq.) in dioxane (5 mL), was stirred under N₂, then Pd(OAc)₂ (6 mg, 29 μmol, 0.1 eq.) and XantPhos (33 mg, 58 μmol, 0.2 eq.) were added, and the resulting mixture was stirred at 100° C. for additional 12 h. After completion, the reaction mixture was diluted with H₂O (10 mL), extracted with EtOAc (10 mL×3). The organic layers were combined and washed with brine (10 mL), dried over anhydrous Na₂SO₄, and concentrated under reduced pressure. The residue was purified by flash column chromatography on silica gel to afford tert-butyl ((3S,6S,10aS)-5-oxo-3-(5-oxo-6-(pyridin-3-yl)-4,6-diazaspiro[2.4]heptane-4-carbonyl)decahydropyrrolo[1,2-a]azocin-6-yl)carbamate (40 mg, 80 μmol, 28%) as a white solid. LCMS (ESI): m/z=498 [M+H]⁺.

### Synthesis of (3S,6S,9aS)-3-{8-acetyl-5-oxa-2,8-diazaspiro[3.5]nonane-2-carbonyl}-6-amino-octahydro-1H-pyrrolo[1,2-a]azepin-5-one trifluoroacetic salt

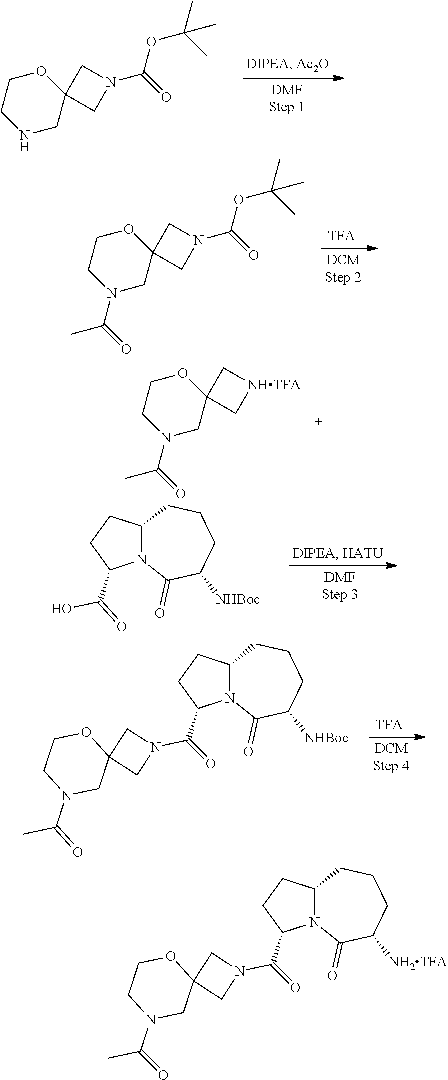

### Step 1: Preparation of Tert-Butyl 8-acetyl-5-oxa-2,8-diazaspiro[3.5]nonane-2-carboxylate

To an ice-bath cooled solution tert-butyl 5-oxa-2,8-diazaspiro[3.5]nonane-2-carboxylate (40 mg, 175 μmol, 1 eq) and N,N-diisopropylethylamine (152 μL, 875 μmol, 5 eq) in methylene chloride (2.5 mL) at 0° C. was slowly added acetic anhydride (26.7 mg, 262 μmol, 1.5 eq) in methylene chloride (2.5 mL). The mixture was stirred at 0° C. for 5 min. then 2 h at room temperature. The reaction mixture was concentrated under reduced pressure. The crude residue was purified by reverse phase chromatography on a C₁₈ cartridge eluting with 5-60% MeCN in water to give tert-butyl 8-acetyl-5-oxa-2,8-diazaspiro[3.5]nonane-2-carboxylate (44.5 mg, 94.0%) as a colorless oil. LCMS (ESI) m/z=271.1 [M+H]⁺.

### Step 2: Preparation of 1-(5-oxa-2,8-diazaspiro[3.5]nonan-8-yl)ethan-1-one

To a solution of a tert-butyl 8-acetyl-5-oxa-2,8-diazaspiro[3.5]nonane-2-carboxylate (44.5 mg, 164 μmol, 1 eq) in methylene chloride (2.5 mL) was added TFA (0.5 mL). The reaction was stirred at room temperature for 1 h. The reaction was concentrated under reduced pressure to give 1-(5-oxa-2,8-diazaspiro[3.5]nonan-8-yl)ethan-1-one (57 mg, 99%, TFA salt) as a thick clear oil. LCMS (ESI) m/z=171.2 [M+H]⁺.

### Step 3: Preparation of Tert-Butyl ((3S,6S,9aS)-3-(8-acetyl-5-oxa-2,8-diazaspiro[3.5]nonane-2-carbonyl)-5-oxooctahydro-1H-pyrrolo[1,2-a]azepin-6-yl)carbamate

To a solution of 1-(5-oxa-2,8-diazaspiro[3.5]nonan-8-yl)ethan-1-one (57 mg, 164 μmol, 1.1 eq, TFA salt) in N,N-dimethylformamide (3 mL) were respectively added N,N-diisopropylethylamine (258 μL, 1.49 mmol, 10 eq), (3S,6S,9aS)-6-((tert-butoxycarbonyl)amino)-5-oxooctahydro-1H-pyrrolo[1,2-a]azepine-3-carboxylic acid (46.5 mg, 149 μmol, 1 eq) and HATU (62.3 mg, 164 μmol, 1.1 eq). The reaction was stirred 2 h at room temperature. The reaction mixture was then directly purified by reverse phase chromatography on a C₁₈ cartridge eluting with 5-60% MeCN in water to give tert-butyl ((3S,6S,9aS)-3-(8-acetyl-5-oxa-2,8-diazaspiro[3.5]nonane-2-carbonyl)-5-oxooctahydro-1H-pyrrolo[1,2-a]azepin-6-yl)carbamate (63.0 mg, 91.0%) as a white solid. LCMS (ESI) m/z=365.2 (M-Boc+2H)⁺.

### Step 4: (3S,6S,9aS)-3-{8-acetyl-5-oxa-2,8-diazaspiro[3.5]nonane-2-carbonyl}-6-amino-octahydro-1H-pyrrolo[1,2-a]azepin-5-one trifluoroacetic salt

To a solution of tert-butyl ((3S,6S,9aS)-3-(8-acetyl-5-oxa-2,8-diazaspiro[3.5]nonane-2-carbonyl)-5-oxooctahydro-1H-pyrrolo[1,2-a]azepin-6-yl)carbamate (33 mg, 71.0 μmol, 1 eq) in methylene chloride (2.5 mL) was added TFA (0.5 mL). The reaction was stirred at room temperature for 1 h. The reaction was concentrated under reduced pressure to give (3S,6S,9aS)-3-(8-acetyl-5-oxa-2,8-diazaspiro[3.5]nonane-2-carbonyl)-6-aminooctahydro-5H-pyrrolo[1,2-a]azepin-5-one (33 mg, 99%, TFA salt) as a thick clear oil. LCMS (ESI) m/z=365.4 [M+H]⁺.

### Synthesis of 4-(azetidin-3-yl)pyridine-2-carbonitrile TFA salt

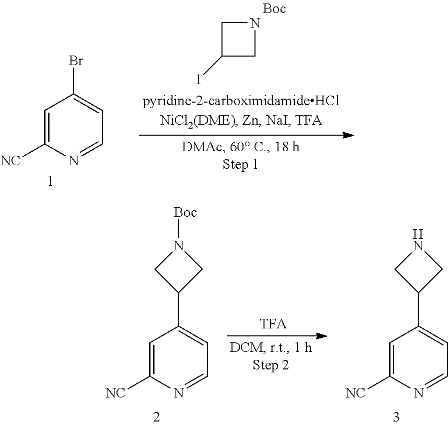

### Step 1: Preparation of Tert-Butyl 3-(2-cyanopyridin-4-yl)azetidine-1-carboxylate

A flame dried tube was charged with a mixture of 4-bromopyridine-2-carbonitrile (100 mg, 0.54 mmol, 1 eq), tert-butyl 3-iodoazetidine-1-carboxylate (185 mg, 0.66 mmol, 1.2 eq), nickel(II) chloride ethylene glycol dimethyl ether complex (11.9 mg, 0.055 mmol, 0.1 eq), pyridine-2-carboximidamide hydrochloride (8.60 mg, 0.055 mmol, 0.1 eq), sodium iodide (40.9 mg, 0.27 mmol, 0.5 eq), pre-activated zinc (71.2 mg, 1.09 mmol, 2.0 eq) and TFA (5 μL, 0.055 mmol, 0.1 eq) in N,N-dimethylacetamide (2.72 mL). The reaction mixture was degassed with nitrogen for 20 min. and the tube was sealed and placed in a pre-equilibrated bath at 60° C. The reaction mixture was stirred for 18 h. The mixture was then filtered through a pad of Celite and washed with EtOAc. The filtrate was diluted with water and the product was extracted with EtOAc (3 ×). The combined organic layers were washed with brine (2 ×), dried with anhydrous Na₂SO₄, filtered and concentrated under vacuum. The crude product was purified by reverse phase chromatography on a 50 g C₁₈ cartridge using a gradient of 5-95% MeCN in water (with 0.1% formic acid) to give tert-butyl 3-(2-cyanopyridin-4-yl)azetidine-1-carboxylate (78 mg, 55%) as a yellow oil. LCMS (ESI): m/z=260.2 [M+H]⁺. ¹H NMR (400 MHz, CDCl₃) δ 8.70 (d, J=4.9 Hz, 1H), 7.69-7.65 (m, 1H), 7.49 (dd, J=5.0, 1.6 Hz, 1H), 4.40 (t, J=8.7 Hz, 2H), 3.94 (dd, J=8.8, 5.6 Hz, 2H), 3.82-3.68 (m, 1H), 1.48 (s, 9H).

### Step 2: Preparation of 4-(azetidin-3-yl)pyridine-2-carbonitrile

TFA (1 mL, 13 mmol, 45 eq) was added to a solution of tert-butyl 3-(2-cyanopyridin-4-yl)azetidine-1-carboxylate (78 mg, 0.30 mmol, 1 eq) in methylene chloride (3 mL). The reaction mixture was stirred at room temperature for 1 h. The reaction mixture was concentrated under vacuum to give 4-(azetidin-3-yl)pyridine-2-carbonitrile (48.2 mg, quant., TFA salt) as a yellow oil. The crude product was used in the next step without further purification. LCMS (ESI): m/z=160.2 [M+H]⁺.

### Synthesis of 4-(azetidin-3-yl)-2-fluoro-3-methoxypyridine

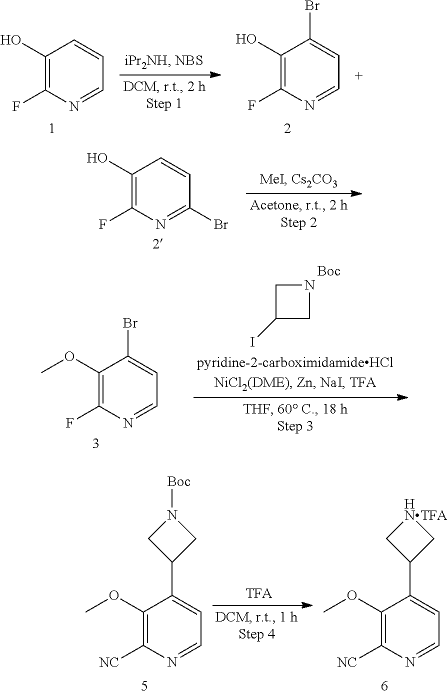

### Step 1: Preparation of 4-bromo-2-fluoropyridin-3-ol

In a dried flask under nitrogen was dissolved 2-fluoropyridin-3-ol (500 mg, 4.42 mmol, 1 eq) in methylene chloride (22.1 mL). Diisopropylamine (0.9 mL, 7.07 mmol, 1.6 eq) was slowly added at room temperature followed by N-bromosuccinimide (786 mg, 4.42 mmol, 1.0 eq). The reaction mixture was stirred at room temperature for 2 h. The LCMS showed a 1:1 mixture of 4-bromo and 6-bromo regioisomers. The solvent was removed under vacuum and the crude product was purified by reverse phase chromatography on a 50 g C18 cartridge using a gradient of 5-95% MeCN in water (with 0.1% formic acid) to give 4-bromo-2-fluoropyridin-3-ol (224 mg, 26.4%) as a yellow solid. LCMS (ESI): m/z=192.0 [M+H]⁺.

¹H NMR (400 MHz, CDCl₃) δ 7.62 (dd, J=5.3, 1.3 Hz, 1H), 7.34 (d, J=5.1 Hz, 1H). 6-Bromo-2-fluoropyridin-3-ol (112 mg, 13.2%) was also isolated.

### Step 2: Preparation of 4-bromo-2-fluoro-3-methoxypyridine

Cesium carbonate (755 mg, 2.32 mmol, 2.0 eq) was added to a solution of 4-bromo-2-fluoropyridin-3-ol (224 mg, 1.16 mmol, 1 eq) in acetone (5.8 mL). Methyl iodide (79 μL, 1.27 mmol, 1.1 eq) was then added and the reaction mixture was stirred at room temperature for 2 h. Acetone was removed under vacuum and the residue was diluted in DCM and water. The aqueous layer was extracted three times with DCM. The combined organic layers were dried with Na2SO4, filtered and concentrated under vacuum to give 4-bromo-2-fluoro-3-methoxypyridine (193 mg, 81.0%) as a yellow liquid (volatile!). The product was used in the next step without further purification. ¹H NMR (400 MHz, CDCl₃) δ 7.73 (dd, J=5.1, 1.5 Hz, 1H), 7.37 (d, J=5.4 Hz, 1H), 4.01 (d, J=2.0 Hz, 3H).

### Step 3: Preparation of tert-butyl 3-(2-fluoro-3-methoxypyridin-4-yl)azetidine-1-carboxylate

A flame dried tube was charged with a mixture of 4-bromo-2-fluoro-3-methoxypyridine (194 mg, 0.94 mmol, 1 eq), tert-butyl 3-iodoazetidine-1-carboxylate (266 mg, 0.94 mmol, 1.2 eq), nickel(II) chloride ethylene glycol dimethyl ether complex (20.6 mg, 0.094 mmol, 0.1 eq), pyridine-2-carboximidamide hydrochloride (14.8 mg, 0.094 mmol, 0.1 eq), sodium iodide (70.4 mg, 0.47 mmol, 0.5 eq), pre-activated zinc (122 mg, 1.88 mmol, 2.0 eq) and TFA (7 μL, 0.094 mmol, 0.1 eq) In THF (4.70 mL). The reaction mixture was degassed with nitrogen for 20 min. and the tube was sealed and placed in a pre-equilibrated bath at 80° C. The reaction mixture was stirred for 18 h. The mixture was filtered through a pad of Celite and washed with EtOAc. The filtrate was diluted with water and the product was extracted with EtOAc (3×). The combined organic layers were washed two times with brine, dried with anhydrous Na₂SO₄ and concentrated under vacuum. The crude product was purified by normal phase chromatography on a 40 g silica gel cartridge using a gradient of 0-100% EtOAc in heptane to give tert-butyl 3-(2-fluoro-3-methoxypyridin-4-yl)azetidine-1-carboxylate (27.9 mg, 10.5%) as a yellow oil. LCMS (ESI): m/z=283.2 [M+H]⁺.

### Step 4: Preparation of 4-(azetidine-3-yl)-2-fluoro-3-methoxypyridine

TFA (1 mL, 13 mmol, 130 eq) was added to a solution of tert-butyl 3-(2-fluoro-3-methoxypyridin-4-yl)azetidine-1-carboxylate (28 mg, 0.99 mmol, 1 eq) in methylene chloride (1 mL). The reaction mixture was stirred at room temperature for 1 h. The reaction mixture was concentrated under vacuum to give the TFA salt of 4-(azetidine-3-yl)-2-fluoro-3-methoxypyridine (18 mg, quant., TFA salt) as a yellow oil. The crude product was used in the next step without further purification. LCMS (ESI): m/z=183.2 [M+H]⁺.

### Synthesis of 3-(azetidin-3-yl)-4-[(2S)-2-(methoxymethyl)azetidin-1-yl]pyridine

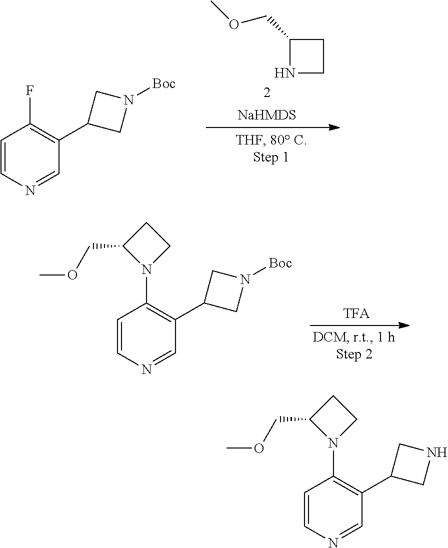

### Step 1: Preparation of Tert-Butyl (S)-3-(4-(2-(methoxymethyl)azetidin-1-yl)pyridin-3-yl)azetidine-1-carboxylate

In a flame dried tube, a solution of NaHMDS (1 M in THF) (1.18 mL, 1.18 mmol, 3.0 eq) was added to a solution of (2S)-2-(methoxymethyl)azetidine (48.0 mg, 0.47 mol, 1.2 eq) in THF (1.97 mL) at room temperature. The reaction was stirred for 5 min. and tert-butyl 3-(4-fluoropyridin-3-yl)azetidine-1-carboxylate (100 mg, 0.40 mmol, 1 eq) was added. The tube was sealed and placed in a pre-equilibrated bath at 80° C. The reaction mixture was stirred at this temperature for 3 h. The reaction was quenched with H₂O and the aqueous layer was extracted three times with EtOAc. The combined organic layers were dried with Na₂SO₄, filtered and concentrated under vacuum. The crude product was purified by reverse phase chromatography on a 50 g C18 cartridge using a gradient of 5-95% MeCN in water (with 0.1% formic acid) to give tert-butyl (S)-3-(4-(2-(methoxymethyl)azetidin-1-yl)pyridin-3-yl)azetidine-1-carboxylate (27.3 mg, 20.6%). LCMS (ESI): m/z=334.2 [M+H]⁺. ¹H NMR (400 MHz, CDCl₃) δ 8.36 (s, 1H), 8.22 (d, J=6.4 Hz, 1H), 6.80 (d, J=6.1 Hz, 1H), 4.62-4.56 (m, 1H), 4.41-4.35 (m, 2H), 4.32-4.22 (m, 2H), 4.13-4.07 (m, 2H), 4.00-3.94 (m, 2H), 3.85-3.79 (m, 1H), 3.65-3.61 (m, 2H), 3.37 (s, 3H), 1.47 (s, 9H).

### Step 2: Preparation of (S)-3-(azetidin-3-yl)-4-(2-(methoxymethyl)azetidin-1-yl)pyridine

TFA (1 mL, 13 mmol, 150 eq) was added to a solution of tert-butyl (S)-3-(4-(2-(methoxymethyl)azetidin-1-yl)pyridin-3-yl)azetidine-1-carboxylate (27 mg, 0.86 mmol, 1 eq) in methylene chloride (1 mL). The reaction mixture was stirred at room temperature for 1 h. The reaction mixture was concentrated under vacuum to give (S)-3-(azetidin-3-yl)-4-(2-(methoxymethyl)azetidin-1-yl)pyridine (20 mg, quant., TFA salt) as a yellow oil. The crude product was used in the next step without further purification. LCMS (ESI): m/z=234.2 [M+H]⁺.

### Synthesis of tert-butyl N-[(3S,6S,9aS)-3-[3-(2-hydroxypyrimidin-4-yl)azetidine-1-carbonyl]-5-oxo-octahydro-1H-pyrrolo[1,2-a]azepin-6-yl]carbamate

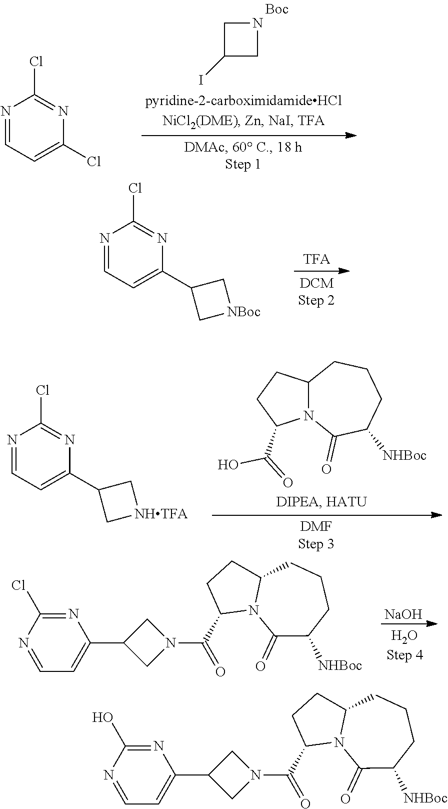

### Step 1: Preparation of Tert-Butyl 3-(2-chloropyrimidin-4-yl)azetidine-1-carboxylate

To a solution of 2,4-dichloropyrimidine (150 mg, 1.00 mmol, 1 eq) and tert-butyl 3-iodoazetidine-1-carboxylate (185 mg, 0.6556 mmol, 1.2 eq) in N,N-dimethylacetamide (5 mL) in a tube were added respectively NaI (74.9 mg, 0.5 mmol, 0.5 eq), pyridine-2-carboximidamide hydrochloride (15.6 mg, 0.1 mmol, 0.1 eq), nickel(II) chloride ethylene glycol dimethyl ether complex (21.9 mg, 0.1 mmol, 0.1 eq), Zn dust (130 mg, 2.00 mmol, 2.0 eq) and TFA (11.4 mg, 0.1 mmol, 0.1 eq). The resulting mixture was degassed with nitrogen for 5 min. The tube was sealed and placed in a pre-equilibrated bath at 60° C. and the reaction mixture was stirred overnight. The mixture was then filtered. The filtrate was diluted with water and the product was extracted with EtOAc (3 ×). The combined organic layers were washed with water (2 ×) and brine (1 ×), dried with sodium sulfate, filtered and concentrated under reduced pressure. The crude product was purified by normal phase chromatography on a 24 g silica gel cartridge eluting with 0-80% EtOAc in heptane to give tert-butyl 3-(2-chloropyrimidin-4-yl)azetidine-1-carboxylate (56.0 mg, 20.8%). 2D NOESY confirmed the desired regiochemistry. LCMS (ESI): m/z=270.1 [M+H]⁺.

### Step 2: Preparation of 4-(azetidin-3-yl)-2-chloropyrimidine

To a solution of tert-butyl 3-(2-chloropyrimidin-4-yl)azetidine-1-carboxylate (56 mg, 0.2076 mmol, 1 eq) in DCM (5 mL) was added TFA (785 μL, 10.3 mmol, 50 eq). The solution was stirred at room temperature for 1 h. The reaction mixture was then concentrated under reduced pressure. Crude TFA salt of 4-(azetidin-3-yl)-2-chloropyrimidine was used as is in the next step. LCMS (ESI): m/z=170.0 [M+H]⁺.

### Step 3: Preparation of tert-butyl N-[(3S,6S,9aS)-3-[3-(2-chloropyrimidin-4-yl)azetidine-1-carbonyl]-5-oxo-octahydro-1H-pyrrolo[1,2-a]azepin-6-yl]carbamate

To a solution of 4-(azetidin-3-yl)-2-chloropyrimidine TFA salt (59 mg, 0.2076 mmol, 1 eq), (3S,6S,9aS)-6-((tert-butoxycarbonyl)amino)-5-oxooctahydro-1H-pyrrolo[1,2-a]azepine-3-carboxylic acid (103 mg, 0.3301 mmol, 1.6 eq) and DIPEA (287 μL, 1.65 mmol, 7.9 eq) in DMF (5 mL) was added HATU (163 mg, 0.4291 mmol, 2.1 eq). The reaction was stirred at room temperature for 1 h. The crude reaction mixture was purified directly by reverse phase chromatography with a 50 g C₁₈ cartridge using a gradient of 5-100% MeCN in water (with 0.1% formic acid) to give tert-butyl N-[(3S,6S,9aS)-3-[3-(2-chloropyrimidin-4-yl)azetidine-1-carbonyl]-5-oxo-octahydro-1H-pyrrolo[1,2-a]azepin-6-yl]carbamate (87.0 mg, 90.3%). LCMS (ESI): m/z=464.2 [M+H]⁺.

### Step 4: Preparation of tert-butyl N-[(3S,6S,9aS)-3-[3-(2-hydroxypyrimidin-4-yl)azetidine-1-carbonyl]-5-oxo-octahydro-1H-pyrrolo[1,2-a]azepin-6-yl]carbamate

To a solution of sodium hydroxide (7.4 mg, 0.1853 mmol, 2 eq) in water (3 mL) was added tert-butyl N-[(3S,6S,9aS)-3-[3-(2-chloropyrimidin-4-yl)azetidine-1-carbonyl]-5-oxo-octahydro-1H-pyrrolo[1,2-a]azepin-6-yl]carbamate (43 mg, 0.09268 mmol, 1 eq). The solution was stirred at room temperature for 5 days. The reaction mixture was then directly purified by reverse phase chromatography with a 50 g C₁₈ cartridge eluting with 5-50% MeCN in water (with 0.1% formic acid) to give tert-butyl N-[(3S,6S,9aS)-3-[3-(2-hydroxypyrimidin-4-yl)azetidine-1-carbonyl]-5-oxo-octahydro-1H-pyrrolo[1,2-a]azepin-6-yl]carbamate (16.0 mg, 50.0%). LCMS (ESI): m/z=446.2 [M+H]⁺.

### Synthesis of 3-(azetidin-3-yl)-4-ethoxy-5-fluoropyridine

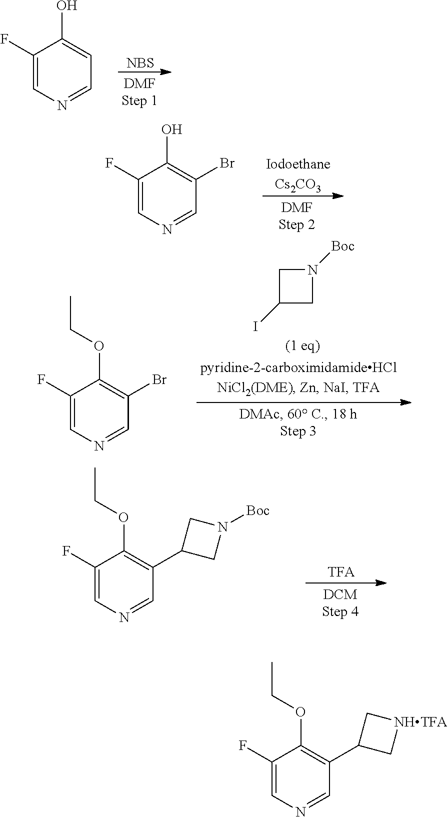

### Step 1: Preparation of 3-bromo-5-fluoropyridin-4-ol

In a solution of 3-fluoropyridin-4-ol (1 g, 8.84 mmol, 1 eq) in N,N-dimethylformamide (8 mL) was added N-bromosuccinimide (1.57 g, 8.84 mmol, 1.0 eq). The reaction mixture was stirred at room temperature for 2 h. The crude product 3-bromo-5-fluoropyridin-4-ol in DMF was used for the next step without further purification. LCMS (ESI): m/z=192.0 [M+H]⁺.

### Step 2: Preparation of 3-bromo-4-ethoxy-5-fluoropyridine

Cesium carbonate (5.40 g, 16.6 mmol, 2.0 eq) was added to a solution of 3-bromo-5-fluoropyridin-4-ol (1.6 g, 8.33 mmol, 1 eq) and iodoethane (2.58 g, 16.6 mmol, 2 eq) in N,N-dimethylformamide (6 mL). The reaction mixture was stirred at room temperature for 18 h. The crude product was purified directly by reverse phase chromatography with a 100 g C₁₈ cartridge using a gradient of 0-10% MeCN in water to give 3-bromo-4-ethoxy-5-fluoropyridine (858 mg, 46.8%) as a brown oil. LCMS (ESI): m/z=220.0 [M+H]⁺. ¹H NMR (400 MHz, CDCl₃) δ 7.73 (d, J=2.2 Hz, 1H), 7.48 (dd, J=6.0, 2.3 Hz, 1H), 3.94 (q, J=7.3 Hz, 2H), 1.52 (t, J=7.3 Hz, 3H).

### Step 3: Preparation of tert-butyl 3-(4-ethoxy-5-fluoropyridin-3-yl)azetidine-1-carboxylate

In a sealed tube, a mixture of 3-bromo-4-ethoxy-5-fluoropyridine (160 mg, 636 μmol, 1 eq), tert-butyl 3-iodoazetidine-1-carboxylate (180 mg, 636 μmol, 1 eq), nickel(II) chloride ethylene glycol dimethyl ether complex (13.9 mg, 63.6 μmol, 0.1 eq), pyridine-2-carboximidamide hydrochloride (10.0 mg, 63.6 μmol, 0.1 eq), sodium iodide (47.6 mg, 318 μmol, 0.5 eq), zinc (83.0 mg, 1.27 mmol, 2.0 eq) and TFA (7.25 mg, 63.6 μmol, 0.1 eq) in N,N-dimethylacetamide (3.5 mL) was degassed with N₂ for 20 min. The tube was placed in a pre-equilibrated bath at 60° C. and the reaction mixture was stirred for 18 h. The mixture was filtered. The crude product was purified by reverse phase chromatography on a 50 g C₁₈ cartridge eluting with 5-100% MeCN in water (with 0.1% formic acid). The combined fractions were concentrated under reduced pressure to give tert-butyl 3-(4-ethoxy-5-fluoropyridin-3-yl)azetidine-1-carboxylate (152 mg, 48.4%, 60% purity) as a white solid. LCMS (ESI): m/z=297.2 [M+H]+.

### Step 4: Preparation of 3-(azetidin-3-yl)-4-ethoxy-5-fluoropyridine

To a solution of tert-butyl 3-(4-ethoxy-5-fluoropyridin-3-yl)azetidine-1-carboxylate (150 mg, 303 μmol) in dichloromethane (1 mL) was added TFA (0.5 mL). The reaction mixture was stirred at room temperature for 2 h. The reaction mixture was concentrated under reduced pressure to give 3-(azetidin-3-yl)-4-ethoxy-5-fluoropyridine (TFA salt). The crude TFA salt was used directly for the next step without further purification. LCMS (ESI): m/z=197.2 [M+H]+.

### Synthesis of 4-(azetidine-3-yl)-3-methoxypyridine-2-carbonitrile

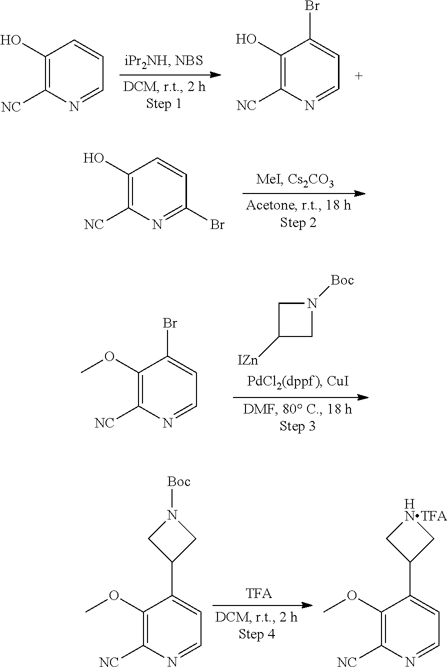

### Step 1: Preparation of 4-bromo-3-hydroxypyridine-2-carbonitrile

In a dried flask under nitrogen was dissolved 3-hydroxypyridine-2-carbonitrile (1.14 g, 9.49 mmol, 1 eq) in methylene chloride (47.1 mL). Diisopropylamine (2.1 mL, 15.1 mmol, 1.6 eq) was slowly added at room temperature followed by N-bromosuccinimide (1.68 g, 9.49 mmol, 1.0 eq). The reaction mixture was stirred at room temperature for 2 h. The LCMS showed a 1:3 mixture of 4-bromo and 6-bromo regioisomers. The solvent was removed under vacuum and the crude product was purified by reverse phase chromatography on a 150 g C₁₈ cartridge using a gradient of 5-95% MeCN in water (with 0.1% formic acid) to give 4-bromo-3-hydroxypyridine-2-carbonitrile (241 mg, 29%) as a yellow solid. ¹H NMR (400 MHz, CDCl₃) δ 8.13 (d, J=5.1 Hz, 1H), 7.69 (d, J=4.9 Hz, 1H). LCMS (ESI): m/z=199.0 [M+H]⁺. 6-Bromo-3-hydroxypyridine-2-carbonitrile (610 mg, 32%) was also isolated.

### Step 2: Preparation of 4-bromo-3-methoxypyridine-2-carbonitrile

Cesium carbonate (788 mg, 2.42 mmol, 2.0 eq) was added to a solution of 4-bromo-3-hydroxypyridine-2-carbonitrile (241 mg, 1.21 mmol, 1 eq) in acetone (6.1 mL). Methyl iodide (165 μL, 2.66 mmol, 1.1 eq) was then added and the reaction mixture was stirred at room temperature for 2 h. Acetone was removed under vacuum and the residue was absorbed on silica gel. The crude product was purified by normal phase chromatography on a 40 g silica gel cartridge using a gradient of 0-100% EtOAc in heptane to give 4-bromo-3-methoxypyridine-2-carbonitrile (70.9 mg, 27.5%) as a white solid. ¹H NMR (400 MHz, CDCl₃) δ 8.25 (d, J=5.1 Hz, 1H), 7.75 (d, J=5.1 Hz, 1H), 4.16 (s, 3H). LCMS (ESI): m/z=213.0 [M+H]⁺.

### Step 3: Preparation of tert-butyl 3-(2-cyano-3-methoxypyridin-4-yl)azetidine-1-carboxylate

A flame-dried round bottom flask was charged with zinc (980 mg, 15 mmol, 5.0 eq.) and anhydrous DMF (1.5 mL). 1,2-Dibromoethane (33.5 μL, 0.40 mmol, 0.15 eq.) was added slowly, followed by TMSCl (50 μL, 0.40 mmol, 0.15 eq.) and the reaction was stirred for 15 min. at room temperature. A solution of N-Boc-3-iodoazetidine (849 mg, 3.0 mmol, 1 eq.) in anhydrous DMF (1.5 mL) was added dropwise. The suspension was stirred at room temperature for 1 h. A flame-dried tube was charged with 4-bromo-3-methoxypyridine-2-carbonitrile (71 mg, 0.33 mmol, 1 eq) in anhydrous DMF (3.32 mL) under nitrogen atmosphere followed by the addition of CuI (6.3 mg, 0.033 mmol, 0.1 eq) and PdCl₂(dppf) (24.4 mg, 0.033 mmol, 0.1 eq). The reaction was degassed with nitrogen for 10 min. and the previously prepared 1 M solution of zincate (0.6 mL, 0.66 mmol, 2.0 eq) was transferred by cannula. The tube was sealed and placed in a pre-equilibrated bath at 80° C. and the reaction was stirred for 18 h. The reaction mixture was cooled down to room temperature, filtered through a pad of Celite and the pad was washed with EtOAc. The filtrate was concentrated under vacuum. The crude product was purified by reverse phase chromatography on a 50 g Cis cartridge using a gradient of 5-95% MeCN in water (with 0.1% formic acid) to give tert-butyl 3-(2-cyano-3-methoxypyridin-4-yl)azetidine-1-carboxylate (65.6 mg, 68.1%). ¹H NMR (400 MHz, CDCl₃) δ 8.45 (d, J=4.9 Hz, 1H), 7.51 (d, J=4.6 Hz, 1H), 4.36 (t, J=8.2 Hz, 2H), 4.14 (s, 2H), 4.07-3.92 (m, 3H), 1.47 (s, 9H). LCMS (ESI): m/z=290.2 [M+H]⁺.

### Step 4: Preparation of 4-(azetidine-3-yl)-3-methoxypyridine-2-carbonitrile

TFA (1 mL, 13 mmol, 52 eq) was added to a solution of tert-butyl 3-(2-fluoro-3-methoxypyridin-4-yl)azetidine-1-carboxylate (65 mg, 0.25 mmol, 1 eq) in methylene chloride (1 mL). The reaction mixture was stirred at room temperature for 1 h. The reaction mixture was concentrated under vacuum to give 4-(azetidine-3-yl)-3-methoxypyridine-2-carbonitrile (47 mg, quant., TFA salt) as a yellow oil. The crude product was used in the next step without further purification. LCMS (ESI): m/z=190.2 [M+H]⁺.

### Synthesis of 3-(azetidin-3-yl)-4-isopropylpyridine

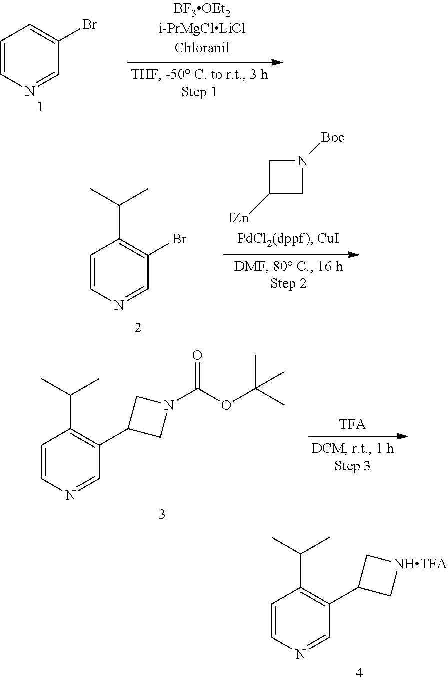

### Step 1: Preparation of 3-bromo-4-(propan-2-yl)pyridine

In a dried flask, boron trifluoride diethyl etherate (492 mg, 3.47 mmol, 1.1 eq) was added to a solution of 3-bromopyridine (500 mg, 3.16 mmol, 1 eq) in THF (6.32 mL) at 0° C. The reaction mixture was stirred 15 min. at 0° C. The reaction mixture was then cooled down to −50° C. and isopropylmagnesium chloride lithium chloride complex solution (1.3 M in THF) (2.9 mL, 3.79 mmol, 1.2 eq) was added dropwise. The reaction mixture was stirred at −50° C. for 30 min. Chloranil (1.55 g, 6.32 mmol, 2 eq) was then added and the mixture was warmed up to room temperature. The mixture was stirred for 2 h at room temperature. A 28% aqueous solution of ammonium hydroxide (5 mL) was then added. The product was extracted with Et₂O (3×). The combined organic layers were dried with magnesium sulfate and filtered through a pad of silica gel. The filtrate was concentrated under reduced pressure. The crude residue was purified by normal phase chromatography on a silica gel cartridge eluting with a gradient of 0-100% EtOAc in heptane to afford 3-bromo-4-(propan-2-yl)pyridine (210 mg, 33.2%) as a red brown oil. ¹H NMR (400 MHz, CDCl₃) δ 8.66 (s, 1H), 8.45 (d, J=4.9 Hz, 1H), 7.22 (d, J=5.1 Hz, 1H), 3.33 (m, 1H), 1.27 (d, J=6.8 Hz, 6H). LCMS (ESI): m/z=200.0 [M+H]+.

### Step 2: Preparation of tert-butyl 3-[4-(propan-2-yl)pyridin-3-yl]azetidine-1-carboxylate

A flame-dried round bottom flask was charged with zinc (84.3 mg, 1.29 mmol, 2.0 eq.) and anhydrous N,N-dimethylacetamide (1 mL). 1,2-Dibromoethane (24.2 mg, 129 μmol, 0.2 eq.) was added slowly, followed by TMSCl (14 mg, 129 μmol, 0.2 eq.) and the reaction was stirred for 15 min. at room temperature. A solution of N-Boc-3-iodoazetidine (365 mg, 1.29 mmol, 2 eq.) in anhydrous N,N-dimethylacetamide (1 mL) was added dropwise. The suspension was stirred at room temperature for 1 h. A flame-dried tube was charged with 3-bromo-4-(propan-2-yl)pyridine (130 mg, 0.65 mmol, 1 eq) in anhydrous N,N-dimethylacetamide (2 mL) under nitrogen atmosphere followed by the addition of CuI (12.3 mg, 0.065 mmol, 0.1 eq) and PdCl₂(dppf) (47.5 mg, 0.065 mmol, 0.1 eq). The reaction was degassed with nitrogen for 15 min. and the previously prepared solution of zincate (2 mL, 1.29 mmol, 2.0 eq) was transferred by cannula. The tube was sealed and placed in a pre-equilibrated bath at 80° C. and the reaction was stirred for 16 h. The reaction mixture was cooled down to room temperature, filtered through a pad of Celite and the pad was washed with EtOAc. The filtrate was concentrated under vacuum. The crude product was purified by reverse phase chromatography on a 50 g C18 cartridge using a gradient of 5-95% MeCN in water (with 0.1% formic acid) to give tert-butyl 3-[4-(propan-2-yl)pyridin-3-yl]azetidine-1-carboxylate (64.9 mg, 24.2%). ¹H NMR (400 MHz, CDCl₃) δ 8.62 (s, 1H), 8.49 (d, J=5.1 Hz, 1H), 8.21 (br. s., 1H), 4.42-4.33 (m, 2H), 4.12-4.05 (m, 2H), 3.78-3.66 (m, 1H), 3.01-2.92 (m, 1H), 1.48 (s, 9H), 1.23 (d, J=6.6 Hz, 6H). LCMS (ESI): m/z=277.2 [M+H]+.

### Step 3: Preparation of 3-(azetidin-3-yl)-4-isopropylpyridine

TFA (0.73 mL, 9.63 mmol, 41 eq) was added to a solution of tert-butyl 3-[4-(propan-2-yl)pyridin-3-yl]azetidine-1-carboxylate (65 mg, 0.235 mmol, 1 eq) in methylene chloride (2.3 mL). The reaction mixture was stirred at room temperature for 1 h. The reaction mixture was concentrated under vacuum to give 3-(azetidin-3-yl)-4-isopropylpyridine (68 mg, quant., TFA salt) as a yellow oil. The crude product was used in the next step without further purification. LCMS (ESI): m/z=177.2 [M+H]⁺.

### Synthesis of tert-butyl ((3S,6S,10aS)-3-((3S,4R)-3-cyano-4-(2-methoxy-6-methylpyridin-4-yl)pyrrolidine-1-carbonyl)-5-oxodecahydropyrrolo[1,2-a]azocin-6-yl)carbamate, tert-butyl ((3S,6S,10aS)-3-((3R,4S)-3-cyano-4-(2-methoxy-6-methylpyridin-4-yl)pyrrolidine-1-carbonyl)-5-oxodecahydropyrrolo[1,2-a]azocin-6-yl)carbamate, tert-butyl ((3S,6S,10aS)-3-((3S,4R)-3-cyano-4-(6-methyl-2-oxo-1,2-dihydropyridin-4-yl)pyrrolidine-1-carbonyl)-5-oxodecahydropyrrolo[1,2-a]azocin-6-yl)carbamate, and tert-butyl ((3S,6S,10aS)-3-((3R,4S)-3-cyano-4-(6-methyl-2-oxo-1,2-dihydropyridin-4-yl)pyrrolidine-1-carbonyl)-5-oxodecahydropyrrolo[1,2-a]azocin-6-yl)carbamate

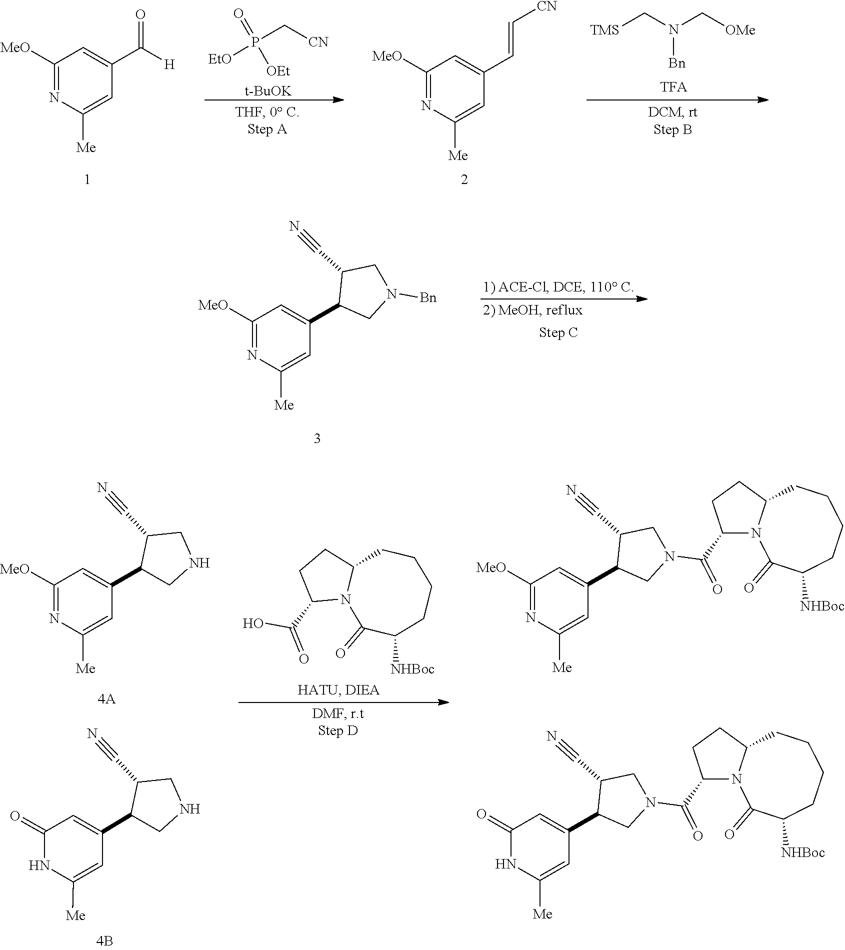

### Step A: Preparation of (E)-3-(2-methoxy-6-methylpyridin-4-yl)acrylonitrile

To a solution of diethyl (cyanomethyl)phosphonate (102 mg, 581 μmol, 1.1 eq.) in THF (16 mL) was added t-BuOK (59 mg, 529 μmol, 1.0 eq.) at 0° C. The solution was stirred at 0° C. for 10 min, then 2-methoxy-6-methylisonicotinaldehyde (80 mg, 529 μmol, 1.0 eq.) was added to the solution at 0° C. the solution was stirred at 0° C. for 10 min. After completion, the reaction mixture was diluted with H₂O (10 mL), extracted with EtOAc (10 mL×3). The organic layers were combined and washed with brine (10 mL), dried over anhydrous Na₂SO₄, and concentrated under reduced pressure to afford (E)-3-(2-methoxy-6-methylpyridin-4-yl)acrylonitrile (135 mg, quant.) as a white solid which was used directly in next step without further purification. LCMS (ESI): m/z=175 [M+H]⁺.

### Step B: Preparation of racemic-(3S,4R)-1-benzyl-4-(2-methoxy-6-methylpyridin-4-yl)pyrrolidine-3-carbonitrile

To a solution of (E)-3-(2-methoxy-6-methylpyridin-4-yl)acrylonitrile (183 mg, 774 μmol, 1 eq.) in DCM (10 mL) benzyl(methoxymethyl)[(trimethylsilyl)methyl]amine (183 mg, 774 μmol, 1 eq.) was added TFA (0.02 mL) at room temperature. The solution was stirred for at 25° C. for 40 min. After completion, the reaction mixture was diluted with H₂O (10 mL), extracted with EtOAc (10 mL×3). The organic layers were combined and washed with brine (10 mL), dried over anhydrous Na₂SO₄, and concentrated under reduced pressure to afford racemic-(3S,4R)-1-benzyl-4-(2-methoxy-6-methylpyridin-4-yl)pyrrolidine-3-carbonitrile (230 mg, quant.) as a white solid, which was used directly in next step without further purification. LCMS (ESI): m/z=308 [M+H]+.

### Step C: Preparation of racemic-(3S,4R)-4-(2-methoxy-6-methylpyridin-4-yl)pyrrolidine-3-carbonitrile and racemic-(3S,4R)-4-(6-methyl-2-oxo-1,2-dihydropyridin-4-yl)pyrrolidine-3-carbonitrile

To a solution of racemic-(3S,4R)-1-benzyl-4-(2-methoxy-6-methylpyridin-4-yl)pyrrolidine-3-carbonitrile (230 mg, 0.75 mmol, 1 eq.) in DCE (5 mL) was added 1-chloroethyl carbonochloridate (536 mg, 3.8 mmol, 5 eq.). The solution was stirred at 110° C. for 24 h, then concentrated under vacuum, the crude product was dissolved in MeOH (5 mL), the solution was stirred at 60° C. for 1 h. LCMS show that the reaction was completed and DP found. the solution was cooled to room temperature, then under reduced pressure to afford a mixture of racemic-(3S,4R)-4-(2-methoxy-6-methylpyridin-4-yl)pyrrolidine-3-carbonitrile and racemic-(3S,4R)-4-(6-methyl-2-oxo-1,2-dihydropyridin-4-yl)pyrrolidine-3-carbonitrile (mixture of 4A, 4B, 230 mg, quant.) as a yellow oil which was used directly in next step without further purification. LCMS (ESI): m/z=218 & 204 [M+H]⁺

### Step D: Preparation tert-butyl ((3S,6S,10aS)-3-((3S,4R)-3-cyano-4-(2-methoxy-6-methylpyridin-4-yl)pyrrolidine-1-carbonyl)-5-oxodecahydropyrrolo[1,2-a]azocin-6-yl)carbamate, tert-butyl ((3S,6S,10aS)-3-((3R,4S)-3-cyano-4-(2-methoxy-6-methylpyridin-4-yl)pyrrolidine-1-carbonyl)-5-oxodecahydropyrrolo[1,2-a]azocin-6-yl)carbamate, tert-butyl ((3S,6S,10aS)-3-((3S,4R)-3-cyano-4-(6-methyl-2-oxo-1,2-dihydropyridin-4-yl)pyrrolidine-1-carbonyl)-5-oxodecahydropyrrolo[1,2-a]azocin-6-yl)carbamate, and tert-butyl ((3S,6S,10aS)-3-((3R,4S)-3-cyano-4-(6-methyl-2-oxo-1,2-dihydropyridin-4-yl)pyrrolidine-1-carbonyl)-5-oxodecahydropyrrolo[1,2-a]azocin-6-yl)carbamate

To a solution of (3S,6S,10aS)-6-((tert-butoxycarbonyl)amino)-5-oxodecahydropyrrolo[1,2-a]azocine-3-carboxylic acid (342 mg, 1.05 mmol, 1 eq.) in DMF (5 mL) were added DIEA (271 mg, 2.1 mmol, 2.0 eq), HATU (441 mg, 1.16 mmol, 1.1 eq.) and afford a mixture of racemic-(3S,4R)-4-(2-methoxy-6-methylpyridin-4-yl)pyrrolidine-3-carbonitrile and racemic-(3S,4R)-4-(6-methyl-2-oxo-1,2-dihydropyridin-4-yl)pyrrolidine-3-carbonitrile (230 mg, 1.05 mmol, 1 eq.). The solution was stirred at 25° C. for 1 hr. After completion, the reaction mixture was purified by prep-HPLC to afford a mixture of tert-butyl ((3S,6S,10aS)-3-((3S,4R)-3-cyano-4-(2-methoxy-6-methylpyridin-4-yl)pyrrolidine-1-carbonyl)-5-oxodecahydropyrrolo[1,2-a]azocin-6-yl)carbamate and tert-butyl ((3S,6S,10aS)-3-((3R,4S)-3-cyano-4-(2-methoxy-6-methylpyridin-4-yl)pyrrolidine-1-carbonyl)-5-oxodecahydropyrrolo[1,2-a]azocin-6-yl)carbamate (94 mg, 178 μmol, 17%) as a yellow oil. LCMS (ESI): m/z=526 [M+H]⁺.

Additionally, tert-butyl ((3S,6S,10aS)-3-((3S,4R)-3-cyano-4-(6-methyl-2-oxo-1,2-dihydropyridin-4-yl)pyrrolidine-1-carbonyl)-5-oxodecahydropyrrolo[1,2-a]azocin-6-yl)carbamate and tert-butyl ((3S,6S,10aS)-3-((3R,4S)-3-cyano-4-(6-methyl-2-oxo-1,2-dihydropyridin-4-yl)pyrrolidine-1-carbonyl)-5-oxodecahydropyrrolo[1,2-a]azocin-6-yl)carbamate (230 mg, 449 μmol, 43%) as a yellow oil. LCMS (ESI): m/z=512 [M+H]+.

### Synthesis of 4-(4-azaspiro[2.4]heptan-6-yl)pyridin-2(1H)-one and 1-methyl-4-(4-azaspiro[2.4]heptan-6-yl)pyridin-2(1H)-one

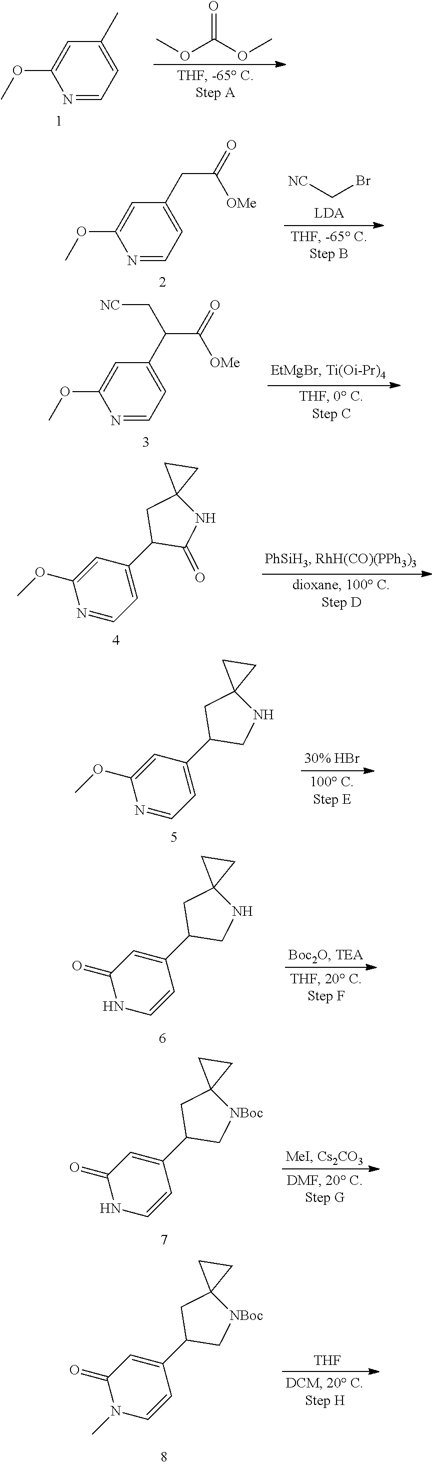

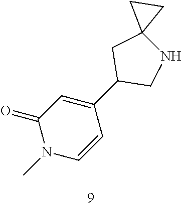

### Step A: methyl 2-(2-methoxypyridin-4-yl)acetate

To a solution of 2-methoxy-4-methylpyridine (1, 50 g, 406 mmol, 1.0 eq.) in THF (500 mL) was added dropwise LDA (243 mL, 485 mmol, 2M, 1.2 eq.) at −65° C. The reaction solution was stirred at −65° C. for 1 h and dimethyl carbonate (40 g, 445 mmol, 1.1 eq.) was added into the reaction mixture. After addition, the mixture was stirred at −65° C. for an additional 1 hr. After completion, the reaction mixture was quenched with saturated NH₄Cl (aq.) solution (500 mL) and extracted with EtOAc (500 mL×3). The organic layers were combined and washed with brine (500 mL), dried over anhydrous Na₂SO₄ and concentrated under reduced pressure. The residue was purified by flash column chromatography on silica gel to afford methyl 2-(2-methoxypyridin-4-yl)acetate (2, 35 g, 192 mmol, 47%) as a yellow oil. LCMS (ESI): m/z=182.2 [M+H]⁺.

¹H NMR (400 MHz, CDCl₃) δ 8.14-8.04 (m, 1H), 6.84-6.77 (m, 1H), 6.67 (s, 1H), 3.96-3.90 (m, 3H), 3.73-3.68 (m, 3H), 3.58 (s, 2H).

### Step B: methyl 3-cyano-2-(2-methoxypyridin-4-yl)propanoate

To a solution of methyl 2-(2-methoxypyridin-4-yl)acetate (2, 35 g, 192 mmol, 1.0 eq.) in THF (500 mL) was added dropwise LDA (148 mL, 296 mmol, 2M, 1.5 eq.) at −65° C. under N₂. The reaction solution was stirred at −65° C. for 1 h and 2-bromoacetonitrile (28.4 g, 239 mmol, 1.2 eq.) was added into the reaction mixture. After addition, the mixture was stirred at −65° C. for an additional 1 hr. After completion, the reaction mixture was quenched with saturated NH₄Cl (aq.) solution (500 mL) and extracted with EtOAc (300 mL×3). The organic layers were combined and washed with brine (500 mL), dried over anhydrous Na₂SO₄ and concentrated under reduced pressure. The residue was purified by flash column chromatography on silica gel to afford methyl 3-cyano-2-(2-methoxypyridin-4-yl)propanoate (3, 39 g, 177 mmol, 92%) as a yellow oil. LCMS (ESI): m/z=221.1 [M+H]⁺.

¹H NMR (400 MHz, CDCl₃) δ 8.18 (d, J=5.3 Hz, 1H), 6.79 (dd, J=5.3, 1.5 Hz, 1H), 6.66 (d, J=1.4 Hz, 1H), 3.94 (s, 3H), 3.88 (t, J=7.6 Hz, 1H), 3.75 (s, 3H), 3.06-2.97 (m, 1H), 2.87-2.77 (m, 1H).

### Step C: 6-(2-methoxypyridin-4-yl)-4-azaspiro[2.4]heptan-5-one

To a solution of methyl 3-cyano-2-(2-methoxypyridin-4-yl)propanoate (3, 39 g, 177 mmol, 1.0 eq.) and Titanium tetraisopropanolate (23.3 g, 82 mmol, 0.5 eq.) in THF (800 mL) was added dropwise EtMgBr (134 mL, 402 mmol, 3M, 2.3 eq.) at 0° C. After addition, the reaction mixture was stirred at 0° C. for 1 hr. After completion, the reaction mixture was quenched with HCl aqueous solution (250 mL, 2N) and filtered, The filtrate was extracted with EtOAc (500 mL×3). The organic layers were combined, dried over anhydrous Na₂SO₄, and concentrated under reduced pressure. The residue was purified by flash column chromatography on silica gel to afford 6-(2-methoxypyridin-4-yl)-4-azaspiro[2.4]heptan-5-one (4, 12 g, 54.8 mmol, 31%) as a white solid. LCMS (ESI): m/z=219.2 [M+H]⁺.

¹H NMR (400 MHz, CDCl₃) δ 8.14 (d, J=5.3 Hz, 1H), 6.88 (dd, J=5.3, 1.4 Hz, 1H), 6.81-6.65 (m, 2H), 3.93 (s, 3H), 3.87-3.78 (m, 1H), 2.58-2.48 (m, 1H), 2.38-2.27 (m, 1H), 0.98-0.92 (m, 1H), 0.91-0.85 (m, 1H), 0.81-0.72 (m, 2H).

### Step D: 6-(2-methoxypyridin-4-yl)-4-azaspiro[2.4]heptane

To a solution of 6-(2-methoxypyridin-4-yl)-4-azaspiro[2.4]heptan-5-one (4, 11 g, 50.2 mmol, 1.0 eq.) and methanidylidyneoxidanium tris(triphenylphosphane) hydrogen rhodium (2.31 g, 2.52 mmol, 0.05 eq.) in dioxane (200 mL) was added phenylsilane (33 g, 306 mmol, 6.0 eq.). The reaction solution was stirred at 100° C. for 12 h. After completion, the reaction mixture was cooled to 20° C. and poured into HCl aqueous solution (100 mL, 2N). The resulting solution was stirred at 20° C. for 1 h and filtered. The filtrate was concentrated under reduced pressure to afford 6-(2-methoxypyridin-4-yl)-4-azaspiro[2.4]heptane (5, 11 g, 53.7 mmol, quant.) as a brown solid, which was used in next step directly without further purification. LCMS (ESI): m/z=205.2[M+H]+. ¹H NMR (400 MHz, CDCl₃) δ 8.08 (d, J=5.3 Hz, 1H), 6.79 (dd, J=5.3, 1.4 Hz, 1H), 6.64 (s, 1H), 3.93 (s, 3H), 3.49-3.36 (m, 2H), 3.03-2.91 (m, 1H), 2.19-2.15 (m, 1H), 1.97-1.87 (m, 1H), 0.85-0.80 (m, 2H), 0.66-0.58 (m, 2H).

### Step E: 4-(4-azaspiro[2.4]heptan-6-yl)pyridin-2(1H)-one (HBr salt)

A solution of 6-(2-methoxypyridin-4-yl)-4-azaspiro[2.4]heptane (5, 11 g, 53.7 mmol, 1.0 eq.) in a mixture of 30% HBr/AcOH (50 mL) and 30% HBr/H₂O (100 mL) was stirred at 100° C. for 12 h. After completion, the reaction mixture was concentrated under reduced pressure. The residue was neutralized carefully with NaHCO₃ (aq.) until the pH was adjusted to pH=7. The mixture was concentrated under reduced pressure to afford crude 4-(4-azaspiro[2.4]heptan-6-yl)pyridin-2(1H)-one (6, 11 g, 57.6 mmol, quant.) as a yellow solid, which was used in next step directly without further purification. LCMS (ESI): m/z=191.2[M+H]+. ¹H NMR (400 MHz, DMSO-d₆) δ 11.48 (s, 1H), 7.37 (d, J=6.8 Hz, 1H), 6.29 (d, J=0.6 Hz, 1H), 6.22 (dd, J=6.8, 1.7 Hz, 1H), 3.71-3.62 (m, 1H), 3.56-3.41 (m, 1H), 3.26-3.19 (m, 1H), 2.24-2.03 (m, 2H), 1.21-1.02 (m, 2H), 0.94-0.78 (m, 2H).

### Step F: tert-butyl 6-(2-oxo-1,2-dihydropyridin-4-yl)-4-azaspiro[2.4]heptane-4-carboxylate

To a mixture of 4-(4-azaspiro[2.4]heptan-6-yl)pyridin-2(1H)-one (6, 11 g, 57.9 mmol, 1.0 eq.) and TEA (15 mL, 108 mmol, 1.9 eq.) in THF (200 mL) was added Boc₂O (20 g, 91.7 mmol, 1.6 eq.). The resulting mixture was stirred at 20° C. for 12 h. After completion, the reaction mixture was concentrated under reduced pressure. The residue was purified by flash column chromatography on silica gel to afford tert-butyl 6-(2-oxo-1,2-dihydropyridin-4-yl)-4-azaspiro[2.4]heptane-4-carboxylate (7, 4 g, 13.7 mmol, 24%) as a yellow solid. LCMS (ESI): m/z=291 [M+H]⁺.

### Step G: tert-butyl 6-(1-methyl-2-oxo-1,2-dihydropyridin-4-yl)-4-azaspiro[2.4]heptane-4-carboxylate

To a solution of tert-butyl 6-(2-oxo-1,2-dihydropyridin-4-yl)-4-azaspiro[2.4]heptane-4-carboxylate (7, 4 g, 13.7 mmol, 1.0 eq.) and Cs₂CO₃ (14 g, 42.9 mmol, 3.1 eq.) in DMF (100 mL) was added Mel (2 mL, 32.1 mmol, 2.3 eq.). The resulting mixture was stirred at 20° C. for 12 h. After completion, the mixture was diluted with water (100 mL) and extracted with EtOAc (150 mL×3). The organic layers were combined and washed with brine (100 mL×2), dried over anhydrous Na₂SO₄, and concentrated under reduced pressure. The residue was purified by flash column chromatography on silica gel to afford tert-butyl 6-(1-methyl-2-oxo-1,2-dihydropyridin-4-yl)-4-azaspiro[2.4]heptane-4-carboxylate (8, 3.0 g, 9.84 mmol, 72%) as a yellow oil. LCMS (ESI) m/z=305 [M+H]⁺.

### Step H: 1-methyl-4-(4-azaspiro[2.4]heptan-6-yl)pyridin-2(1H)-one

To a solution of tert-butyl 6-(1-methyl-2-oxo-1,2-dihydropyridin-4-yl)-4-azaspiro[2.4]heptane-4-carboxylate (8, 3.0 g, 9.84 mmol, 1.0 eq.) in DCM (30 mL) was added TFA (6 mL) and the resulting mixture was stirred at 20° C. for 1 hr. After completion, the mixture was concentrated under reduced pressure to give crude 1-methyl-4-(4-azaspiro[2.4]heptan-6-yl)pyridin-2(1H)-one (9, 2 g, 9.76 mmol, quant.) as a red oil, which was used in next step directly without further purification. LCMS (ESI): m/z=205 [M+H]⁺.

tert-butyl 6-(1-methyl-2-oxo-1,2-dihydropyridin-4-yl)-4-azaspiro[2.4]heptane-4-carboxylate was prepared as described above to afford a racemic mixture. The individual stereoisomers were purified by chiral SFC conditions and absolute configuration was assigned as drawn below, based on the comparison of experimental vibrational circular dichroism (VCD) spectra with theoretical VCD spectra obtained from DFT calculations. Preparative separation method for tert-butyl 6-(1-methyl-2-oxo-1,2-dihydropyridin-4-yl)-4-azaspiro[2.4]heptane-4-carboxylate:

Instrument: Waters Thar 80 preparative SFC; Column: ChiralPak AD, 250×21.2 mm I.D., 5 μm; Mobile phase: A for CO₂ and B for methanol (0.1% NH₃H₂O); Gradient: B 40%; Flow rate: 40 mL/min; Back pressure: 100 bar; Column temperature: 35° C.; Wavelength: 220 nm; Cycle-time: 10 min.

(S)-1-methyl-4-(4-azaspiro[2.4]heptan-6-yl)pyridin-2(1H)-one and (R)-1-methyl-4-(4-azaspiro[2.4]heptan-6-yl)pyridin-2(1H)-one were each prepared with the procedure described for racemic 1-methyl-4-(4-azaspiro[2.4]heptan-6-yl)pyridin-2(1H)-one utilizing the appropriate starting materials.

tert-butyl 6-(2-oxo-1,2-dihydropyridin-4-yl)-4-azaspiro[2.4]heptane-4-carboxylate was prepared as described above to afford a racemic mixture. The individual stereoisomers were purified by chiral SFC conditions and absolute configuration was assigned as drawn below. Assignment of absolute configuration was verified by independent synthesis of enantiopure tert-butyl (S)-6-(1-methyl-2-oxo-1,2-dihydropyridin-4-yl)-4-azaspiro[2.4]heptane-4-carboxylate via alkylation of tert-butyl (S)-6-(2-oxo-1,2-dihydropyridin-4-yl)-4-azaspiro[2.4]heptane-4-carboxylate, and of enantiopure tert-butyl (R)-6-(1-methyl-2-oxo-1,2-dihydropyridin-4-yl)-4-azaspiro[2.4]heptane-4-carboxylate via alkylation of tert-butyl (R)-6-(2-oxo-1,2-dihydropyridin-4-yl)-4-azaspiro[2.4]heptane-4-carboxylate, with the procedures described for racemic tert-butyl 6-(1-methyl-2-oxo-1,2-dihydropyridin-4-yl)-4-azaspiro[2.4]heptane-4-carboxylate.

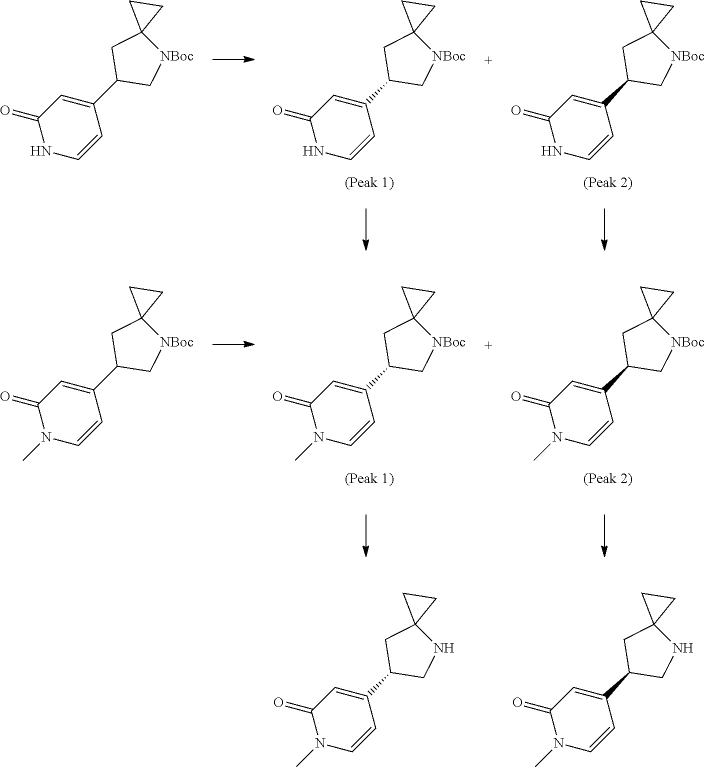

### Preparative Separation Method for tert-butyl 6-(2-oxo-1,2-dihydropyridin-4-yl)-4-azaspiro[2.4]heptane-4-carboxylate

Instrument: SHIMADZU PREP SOLUTION SFC; Column: ChiralPak AD, 250×30 mm I.D., 5 μm; Mobile phase: A for CO₂ and B for methanol (0.1% NH₃H₂O); Gradient: B 40%; Flow rate: 60 mL/min; Back pressure: 100 bar; Column temperature: 35° C.; Wavelength: 220 nm; Cycle-time: 7 min.

TABLE 106

| Name | Structure | LCMS |
|---|---|---|
| tert-butyl (S)-6-(1-methyl-2-oxo-1,2-dihydropyridin- 4-yl)-4-azaspiro[2.4]heptane-4-carboxylate (Peak 1) | 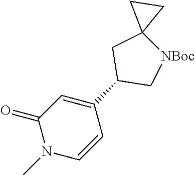 [SMILES: Cn1ccc([C@H]2CN(C(=O)OC(C)(C)C)C3(CC3)C2)cc1=O] | 305 [M + H]⁺ |
| tert-butyl (R)-6-(1-methyl-2-oxo-1,2-dihydropyridin- 4-yl)-4-azaspiro[2.4]heptane-4-carboxylate (Peak 2) | 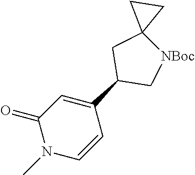 [SMILES: Cn1ccc([C@@H]2CN(C(=O)OC(C)(C)C)C3(CC3)C2)cc1=O] | 305 [M + H]⁺ |
| tert-butyl (S)-6-(2-oxo-1,2-dihydropyridin-4-yl)-4- azaspiro[2.4]heptane-4-carboxylate (Peak 1) | 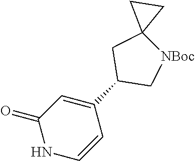 [SMILES: CC(C)(C)OC(=O)N1C[C@H](c2cc[nH]c(=O)c2)CC12CC2] | 291 [M + H]⁺ |
| tert-butyl (R)-6-(2-oxo-1,2-dihydropyridin-4-yl)-4- azaspiro[2.4]heptane-4-carboxylate (Peak 2) | 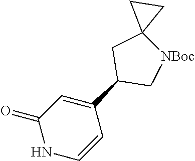 [SMILES: CC(C)(C)OC(=O)N1C[C@@H](c2cc[nH]c(=O)c2)CC12CC2] | 291 [M + H]⁺ |

(S)-4-(4-azaspiro[2.4]heptan-6-yl)pyridin-2(1H)-one and (R)-4-(4-azaspiro[2.4]heptan-6-yl)pyridin-2(1H)-one were each prepared with the procedure described for 1-methyl-4-(4-azaspiro[2.4]heptan-6-yl)pyridin-2(1H)-one utilizing the appropriate starting materials.

### Syntheses of Phosphonic Acid Analogues: General Scheme 3

### Synthesis of (difluoro(2-(((3S,6S,9aS)-5-oxo-3-(5,6,7,8-tetrahydroimidazo[1,5-a]pyrazine-7-carbonyl)octahydro-1H-pyrrolo[1,2-a]azepin-6-yl)carbamoyl)benzo[b]thiophen-5-yl)methyl)phosphon2 ic acid (203)

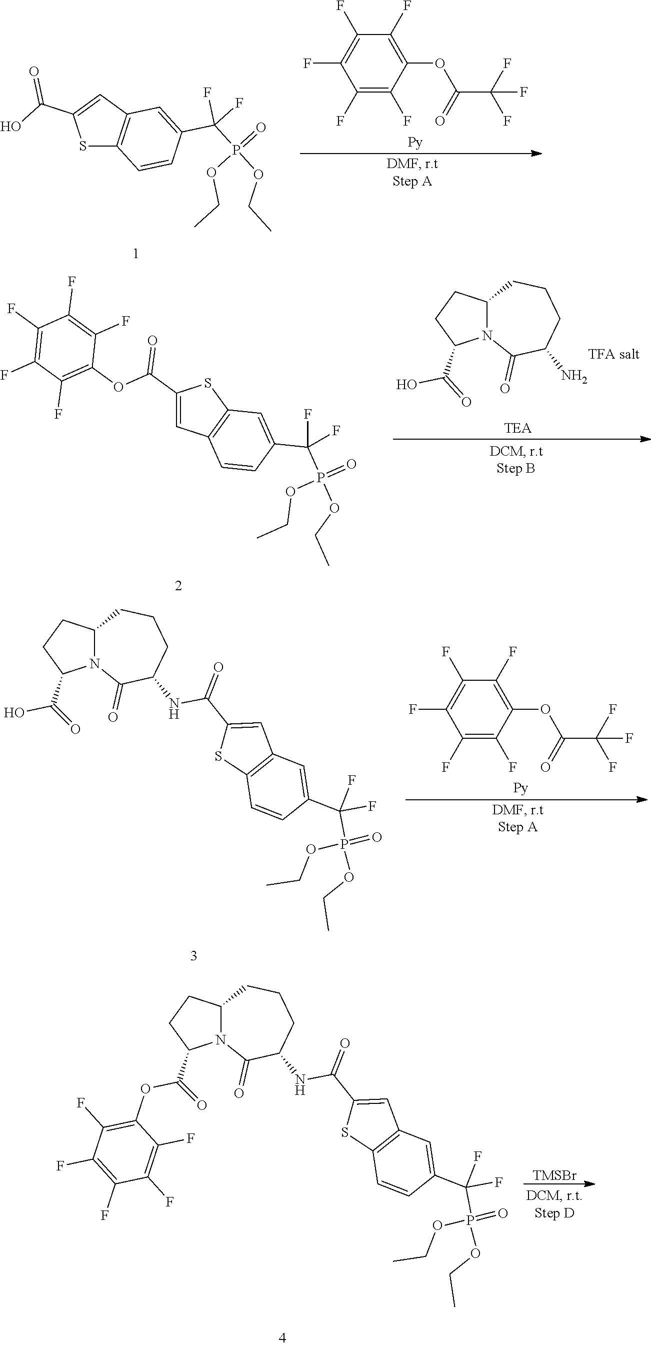

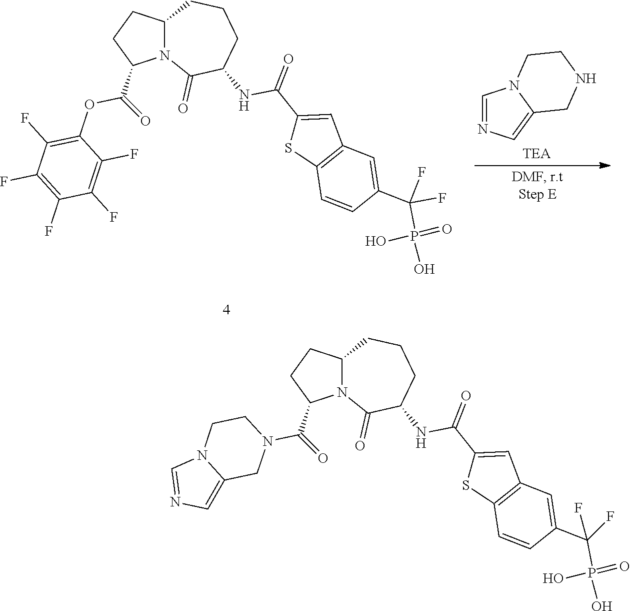 [SMILES: O=C(N[C@H]1CCC[C@H]2CC[C@@H](C(=O)Oc3c(F)c(F)c(F)c(F)c3F)N2C1=O)c1cc2cc(C(F)(F)P(=O)(O)O)ccc2s1 ; c1ncn2c1CNCC2 ; O=C(N[C@H]1CCC[C@H]2CC[C@@H](C(=O)N3CCn4cncc4C3)N2C1=O)c1cc2cc(C(F)(F)P(=O)(O)O)ccc2s1]

### Step A: perfluorophenyl 5-((diethoxyphosphoryl)difluoromethyl)benzo[b]thiophene-2-carboxylate

To a solution of 5-((diethoxyphosphoryl)difluoromethyl)benzo[b]thiophene-2-carboxylic acid (1, 3.64 g, 9.99 mmol, 1 eq.) in DMF (30 mL) were added pyridine (1.60 mL, 19.9 mmol, 2 eq.) and 2,3,4,5,6-pentafluorophenyl 2,2,2-trifluoroacetate (2.55 mL, 14.9 mmol, 1.5 eq.) under N₂ with stirring. The resulting mixture was stirred at room temperature for 1 hr. After completion, the reaction mixture was poured into H₂O (20 mL), then extracted with EtOAc (50 mL×3). The organic layers were combined and washed with brine (50 mL), dried over anhydrous Na₂SO₄, and concentrated under reduced pressure to afford perfluorophenyl 5-((diethoxyphosphoryl)difluoromethyl)benzo[b]thiophene-2-carboxylate (2, 5.20 g, quant.) as a white solid. LCMS (ESI) m/z=531.0 [M+H]⁺.

### Step B: (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylic acid

To a solution of perfluorophenyl 5-((diethoxyphosphoryl)difluoromethyl)benzo[b]thiophene-2-carboxylate (2, 5.3 g, 9.99 mmol, 1 eq.) in DCM (50 mL) were added TEA (4.05 g, 40.1 mmol, 4.0 eq.) and (3S,6S,9aS)-6-amino-5-oxooctahydro-1H-pyrrolo[1,2-a]azepine-3-carboxylic acid (2.3 g, 10.8 mmol, 1.1 eq.) under N₂ with stirring. The resulting mixture was stirred at room temperature for 2 h. After completion, the reaction mixture was poured into H₂O (20 mL), then extracted with EtOAc (50 mL×3). The organic layers were combined and washed with brine (50 mL), dried over anhydrous Na₂SO₄, and concentrated under reduced pressure to afford (3S,6S,9aS)-6-{5-[(diethoxyphosphoryl)difluoromethyl]-1-benzothiophene-2-amido}-5-oxo-octahydro-1H-pyrrolo[1,2-a]azepine-3-carboxylic acid (3, 5.0 g, quant.) as a white solid, which was used in next step directly without further purification. LCMS (ESI) m/z=559.1[M+H]⁺.

### Step C: perfluorophenyl (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylate

To a solution of (3S,6S,9aS)-6-{5-[(diethoxyphosphoryl)difluoromethyl]-1-benzothiophene-2-amido}-5-oxo-octahydro-1H-pyrrolo[1,2-a]azepine-3-carboxylic acid (3, 5.58 g, 9.99 mmol, 1 eq.) in DMF (35 mL) were added pyridine (1.60 mL, 19.9 mmol, 2 eq.) and 2,3,4,5,6-pentafluorophenyl 2,2,2-trifluoroacetate (2.55 mL, 14.9 mmol, 1.5 eq.) under N₂ with stirring. The resulting mixture was stirred at room temperature for 0.5 h. After completion, the reaction mixture was poured into H₂O (20 mL), then extracted with EtOAc (50 mL×3). The organic layers were combined and washed with brine (50 mL), dried over anhydrous Na₂SO₄, and concentrated under reduced pressure. The residue was purified by flash column chromatography on silica gel to afford perfluorophenyl (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylate (4, 5.50 g, 7.59 mmol, 76%) as a white solid. LCMS (ESI) m/z=725.3 [M+H]⁺.

### Step D: (difluoro(2-(((3S,6S,9aS)-5-oxo-3-((perfluorophenoxy)carbonyl)octahydro-1H-pyrrolo[1,2-a]azepin-6-yl)carbamoyl)benzo[b]thiophen-5-yl)methyl)phosphonic acid

To a solution of perfluorophenyl (3S,6S,9aS)-6-(5-((diethoxyphosphoryl)difluoromethyl)benzo[b]thiophene-2-carboxamido)-5-oxooctahydro-1H-pyrrolo[1,2-a]azepine-3-carboxylate (4, 2.5 g, 3.45 mmol, 1 eq.) in DCM (10 mL) was added TMSBr (455 μL, 3.45 mmol, 1.0 eq.) under N₂ with stirring. The resulting mixture was stirred at room temperature overnight. After completion, the reaction mixture was poured into H₂O (20 mL), then extracted with DCM (50 mL×3). The organic layers were combined and washed with brine (50 mL), dried over anhydrous Na₂SO₄, and concentrated under reduced pressure to afford (difluoro(2-(((3S,6S,9aS)-5-oxo-3-((perfluorophenoxy)carbonyl)octahydro-1H-pyrrolo[1,2-a]azepin-6-yl)carbamoyl)benzo[b]thiophen-5-yl)methyl)phosphonic acid (5, 2.10 g, quant.) as a yellow solid, which was used in next step directly without further purification. LCMS (ESI) m/z=669.2 [M+H]⁺.

### Step E: (difluoro(2-(((3S,6S,9aS)-5-oxo-3-(5,6,7,8-tetrahydroimidazo[1,5-a]pyrazine-7-carbonyl)octahydro-1H-pyrrolo[1,2-a]azepin-6-yl)carbamoyl)benzo[b]thiophen-5-yl)methyl)phosphonic acid

To a solution of 5,6,7,8-tetrahydroimidazo[1,5-a]pyrazine (50 mg, 405 μmol, 1 eq.) in DMF (2 mL) were added (difluoro(2-(((3S,6S,9aS)-5-oxo-3-((perfluorophenoxy)carbonyl)octahydro-1H-pyrrolo[1,2-a]azepin-6-yl)carbamoyl)benzo[b]thiophen-5-yl)methyl)phosphonic acid (5, 324 mg, 485 μmol, 1.2 eq.) and TEA (168 μL, 1.21 mmol, 3.0 eq.) under N₂ with stirring. The resulting mixture was stirred at room temperature overnight. After completion, the reaction mixture was poured into H₂O (20 mL), then extracted with EtOAc (50 mL×3). The organic layers were combined and washed with brine (50 mL), dried over anhydrous Na₂SO₄, and concentrated under reduced pressure. The residue was purified by prep-HPLC to afford (difluoro(2-(((3S,6S,9aS)-5-oxo-3-(5,6,7,8-tetrahydroimidazo[1,5-a]pyrazine-7-carbonyl)octahydro-1H-pyrrolo[1,2-a]azepin-6-yl)carbamoyl)benzo[b]thiophen-5-yl)methyl)phosphonic acid (45.2 mg, 74.3 μmol, 18%) as a white solid. ¹H NMR (400 MHz, DMSO) δ 8.80-8.69 (m, 1H), 8.48-8.33 (m, 1H), 8.23-8.19 (m, 1H), 8.04-7.98 (m, 2H), 7.56 (d, J=8.3 Hz, 1H), 7.05 (s, 1H), 5.04-4.92 (m, 2H), 4.65 (t, J=8.7 Hz, 1H), 4.45-4.28 (m, 1H), 4.26-4.00 (m, 4H), 3.86-3.81 (m, 1H), 2.25-1.72 (m, 10H). LCMS (ESI): m/z=608.0 [M+H]⁺.

### Synthesis of S-(2-(((benzylamino)(difluoro(2-(((3S,6S,9aS)-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphoryl)oxy)ethyl) 3-hydroxy-2,2-dimethylpropanethioate (204)

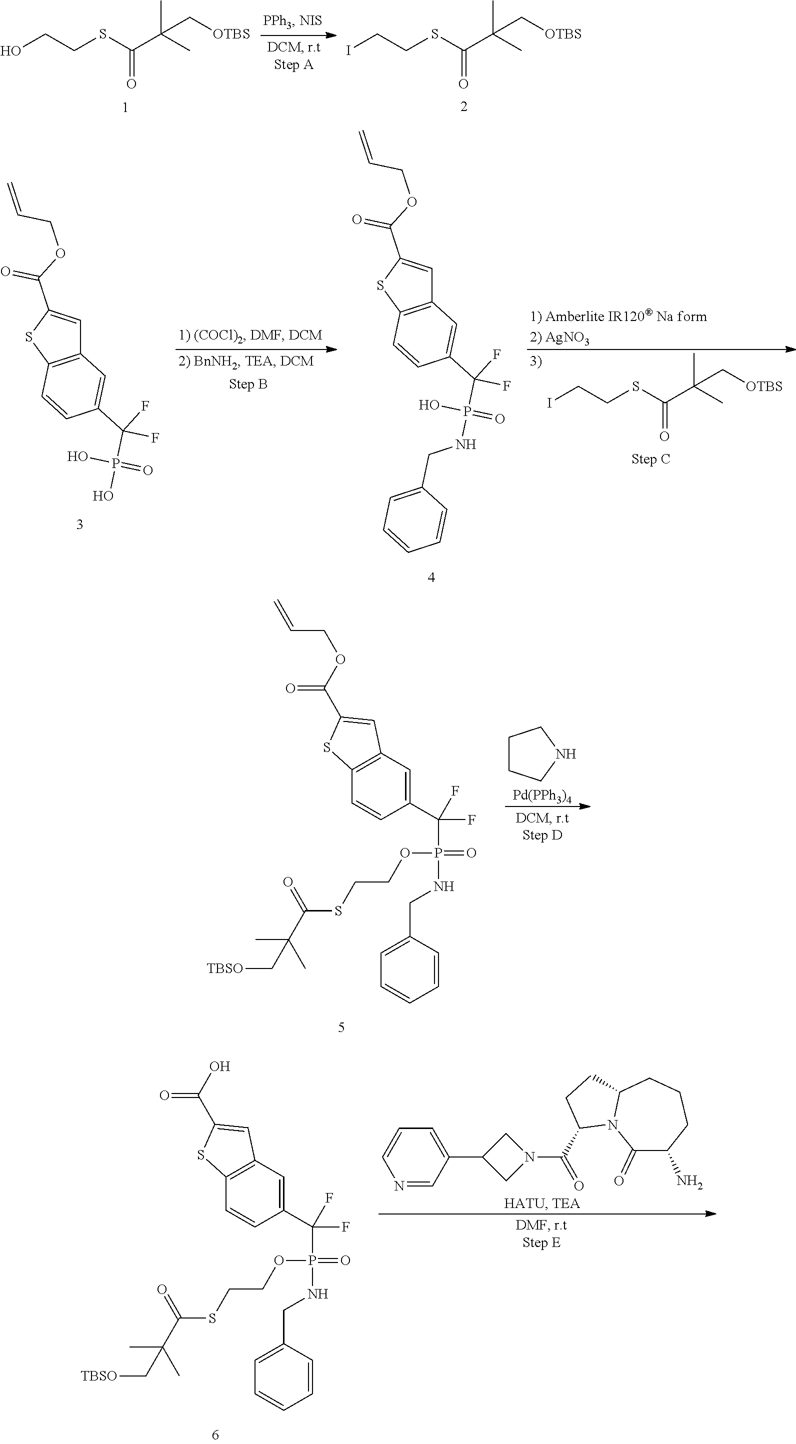

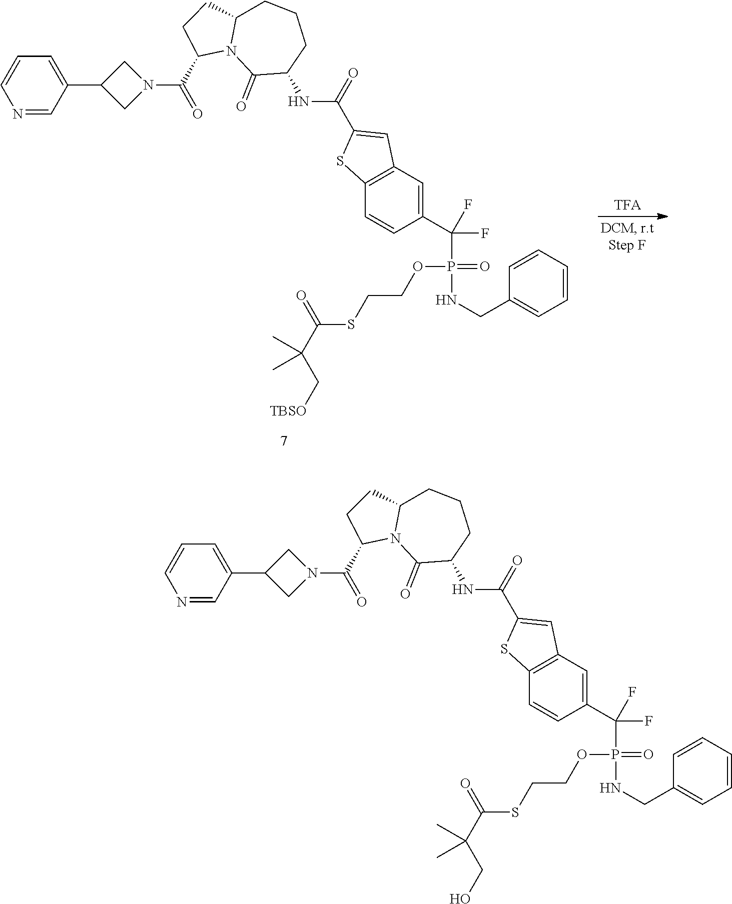 [SMILES: CC(C)(CO[Si](C)(C)C(C)(C)C)C(=O)SCCOP(=O)(NCc1ccccc1)C(F)(F)c1ccc2sc(C(=O)N[C@H]3CCC[C@H]4CC[C@@H](C(=O)N5CC(c6cccnc6)C5)N4C3=O)cc2c1 ; CC(C)(CO)C(=O)SCCOP(=O)(NCc1ccccc1)C(F)(F)c1ccc2sc(C(=O)N[C@H]3CCC[C@H]4CC[C@@H](C(=O)N5CC(c6cccnc6)C5)N4C3=O)cc2c1]

### Step A: S-(2-iodoethyl) 3-((tert-butyldimethylsilyl)oxy)-2,2-dimethylpropanethioate

To a solution of S-(2-hydroxyethyl) 3-((tert-butyldimethylsilyl)oxy)-2,2-dimethylpropanethioate (1, 700 mg, 2.39 mmol, 1 eq.) in DCM (20 mL) was added PPh₃ (626 mg, 2.39 mmol, 1.0 eq.) in portion wise at 0° C. and the resulting mixture was stirred for 30 min. Then NIS (537 mg, 2.39 mmol, 1.0 eq) was added dropwise at 0° C. and the reaction mixture was stirred at 0° C. for 1 h. After completion, the reaction mixture was concentrated under reduced pressure. The residue was purified by flash column chromatography on silica gel to give S-(2-iodoethyl) 3-((tert-butyldimethylsilyl)oxy)-2,2-dimethylpropanethioate (2, 700 mg, 1.73 mmol, 73%) as a clear oil. ¹H NMR (400 MHz, CDCl₃) δ 3.57 (s, 2H), 3.31-3.24 (m, 2H), 3.23-3.15 (m, 2H), 1.17 (s, 6H), 0.84 (s, 9H), 0.00 (s, 6H).

### Step B: P-((2-((allyloxy)carbonyl)benzo[b]thiophen-5-yl)difluoromethyl)-N-benzylphosphonamidic acid

Oxalyl chloride (1.01 g, 8.00 mmol, 5 eq.) was added dropwise to the solution of ((2-((allyloxy)carbonyl)benzo[b]thiophen-5-yl)difluoromethyl)phosphonic acid (3, 150 mg, 0.4307 mmol, 1 eq.) in dry DCM (10 mL) and DMF (1 drop) at 0° C. The reaction mixture was stirred at 40° C. for an additional 2 h. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (10 mL), then added to a mixture of phenylmethanamine (46.0 mg, 430 μmol, 1 eq.) and TEA (217 mg, 2.15 mmol, 5 eq.) in anhydrous DCM (10 mL) at 0° C. The reaction was allowed to warm to 25° C. and stirred for an additional 2 h. After completion, the reaction mixture was concentrated under reduced pressure. The residue was purified by Biotage® C18 column chromatography to give P-((2-((allyloxy)carbonyl)benzo[b]thiophen-5-yl)difluoromethyl)-N-benzylphosphonamidic acid (4, 100 mg, 0.23 mmol, 53%) as a colorless oil. LCMS (ESI): m/z=438 [M+H]⁺.

### Step C: allyl 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylate

NaOH (18.2 mg, 457 μmol, 2 eq) in water (2 mL) was added dropwise to a stirred suspension of P-((2-((allyloxy)carbonyl)benzo[b]thiophen-5-yl)difluoromethyl)-N-benzylphosphonamidic acid (4, 100 mg, 0.23 mmol, 1 eq.) in H₂O (6 mL). When the mixture became clear (pH˜9), silver nitrate (85.2 mg, 502 μmol, 2.2 eq.) was added. After stirring at 0° C. for 2 h, the gray precipitate was collected by filtration and dried under vacuum. The powder was suspended in dry Toluene (1 mL) and S-(2-iodoethyl) 3-((tert-butyldimethylsilyl)oxy)-2,2-dimethylpropanethioate (2, 1.68 g, 6.96 mmol, 30 eq.) was added. The mixture was stirred at 80° C. for 24 h. After filtration, the solvent was removed in vacuo. The crude residue was directly purified by flash column chromatography on silica gel to give allyl 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylate (5, 40.0 mg, 56 μmol, 25%) as a white solid. LCMS (ESI): m/z=734.2 [M+Na]⁺.

### Step D: 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylic acid

To a solution of allyl 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylate (5, 35 mg, 49 μmol, 1 eq.) in DCM (1 mL) were added Pd(PPh₃)₄ (2.83 mg, 2.45 μmol, 0.05 eq.) and pyrrolidine (3 mg, 49.1 μmol, 1 eq.). The mixture was purged and degassed with N₂ (three times), then stirred at 25° C. for 2 h. After completion, the mixture was concentrated under reduced pressure. The residue was purified by Biotage® C18 column chromatography to give 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylic acid (6, 20.0 mg, 30 μmol, 61%) as a colorless oil. LCMS (ESI): m/z=672.1 [M+H]⁺.

### Step E: S-(2-(((benzylamino)(difluoro(2-(((3S,6S,9aS)-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphoryl)oxy)ethyl) 3-((tert-butyldimethylsilyl)oxy)-2,2-dimethylpropanethioate

To a solution of (3S,6S,9aS)-6-amino-3-[3-(pyridin-3-yl)azetidine-1-carbonyl]-octahydro-1H-pyrrolo[1,2-a]azepin-5-one (10 mg, 30 μmol, 1 eq.), TEA (15 mg, 150 μmol, 5 eq.) and 5-(((benzylamino)(2-((3-((tert-butyldimethylsilyl)oxy)-2,2-dimethylpropanoyl)thio)ethoxy)phosphoryl)difluoromethyl)benzo[b]thiophene-2-carboxylic acid (6, 20 mg, 30 μmol, 1 eq.) in DMF (1 mL) was added HATU (12 mg, 30 μmol, 1.0 eq.) and the reaction mixture was stirred at room temperature overnight under N₂. After completion, the reaction mixture was purified by prep-HPLC to afford S-(2-(((benzylamino)(difluoro(2-(((3S,6S,9aS)-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphoryl)oxy)ethyl) 3-((tert-butyldimethylsilyl)oxy)-2,2-dimethylpropanethioate (7, 5 mg, 6 μmol, 20%). LCMS (ESI): m/z=982 [M+H]⁺.

### Step F: S-(2-(((benzylamino)(difluoro(2-(((3S,6S,9aS)-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphoryl)oxy)ethyl) 3-hydroxy-2,2-dimethylpropanethioate

A solution of S-(2-(((benzylamino)(difluoro(2-(((3S,6S,9aS)-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphoryl)oxy)ethyl) 3-((tert-butyldimethylsilyl)oxy)-2,2-dimethylpropanethioate (7, 8 mg, 8 μmol, 1 eq.) in TFA (1 mL) and DCM (3 mL) was stirred at room temperature for 30 min under N₂. After completion, the reaction mixture was concentrated under reduced pressure to afford S-(2-(((benzylamino)(difluoro(2-(((3S,6S,9aS)-5-oxo-3-(3-(pyridin-3-yl)azetidine-1-carbonyl)octahydro-1H-pyrrolo[1,2-a]azepin-6-yl)carbamoyl)benzo[b]thiophen-5-yl)methyl)phosphoryl)oxy)ethyl) 3-hydroxy-2,2-dimethylpropanethioate (6 mg, 7 mmol, 85%) as a white solid. ¹H NMR (400 MHz, CDCl₃) δ 9.16-8.38 (m, 2H), 8.29-7.98 (m, 3H), 7.97-7.76 (m, 2H), 7.74-7.53 (m, 2H), 7.50-7.27 (m, 6H), 5.10-4.39 (m, 5H), 4.31-4.08 (m, 5H), 4.04-3.88 (m, 2H), 3.60-3.33 (m, 3H), 3.07-2.99 (m, 2H), 2.32-2.25 (m, 1H), 2.21-2.15 (m, 1H), 2.11-1.99 (m, 4H), 1.97-1.80 (m, 4H), 1.19-1.08 (m, 6H). LCMS (ESI): m/z=868 [M+H]⁺.

Compounds prepared according to General Scheme 1: Phosphonic acids

The following compounds in Table 107 were prepared according to the representative procedure described above for the synthesis of (difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonic acid (1) and utilizing the appropriate starting materials and

TABLE 107

| | | | LCMS | |
|---|---|---|---|---|
| Compound | Name | Structure | [M + H]⁺ | NMR |
| 205 | ((2-(((3S,6S,9aS)- 3-(3-(4- ethylpyridin-3- yl)azetidine-1- carbonyl)-5- oxooctahydro-1H- pyrrolo[1,2- a]azepin-6- yl)carbamoyl) benzo | 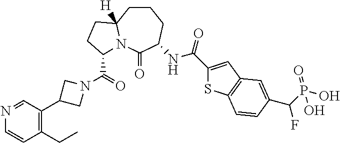 [SMILES: CCc1ccncc1C1CN(C(=O)[C@@H]2CC[C@@H]3CCC[C@H](NC(=O)c4cc5cc(C(F)P(=O)(O)O)ccc5s4)C(=O)N32)C1] | 629.2 | ¹H NMR (400 MHz, CD₃OD) δ 8.78 − 8.67 (m, 1H), 8.42 (d, J = 11.6 Hz, 1H), 8.02 − 7.91 (m, 2H), 7.89 − 7.83 (m, 1H), 7.79 − 7.73 (m, 1H), 7.64 − 7.52 (m, |
| | [b]thiophen-5- | | | 1H), 5.86 − 5.68 |
| | yl)fluoromethyl) | | | (m, 1H), 4.78 − |
| | phosphonic acid | | | 4.71 (m, 2H), |
| | | | | 4.67 − 4.51 (m, |
| | | | | 2H), 4.48 − 4.40 |
| | | | | (m, 1H), 4.35 − |
| | | | | 4.28 (m, 1H), |
| | | | | 4.17 − 4.06 (m, |
| | | | | 2H), 2.85 − 2.75 |
| | | | | (m, 2H), 2.34 − |
| | | | | 1.83 (m, 10H), |
| | | | | 1.30 − 1.26 (m, 3H) |
| 206 | (fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo | 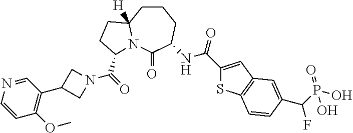 [SMILES: COc1ccncc1C1CN(C(=O)[C@@H]2CC[C@@H]3CCC[C@H](NC(=O)c4cc5cc(C(F)P(=O)(O)O)ccc5s4)C(=O)N32)C1] | 631.0 | ¹H NMR (400 MHz, DMSO- d₆) δ 8.78 − 8.69 (m, 1H), 8.55 − 8.38 (m, 2H), 8.27 − 8.14 (m, 1H), 8.08 − 7.91 (m, 2H), 7.54 − 7.50 (m, 1H), |
| | [b]thiophen-5- | | | 7.29 − 7.16 (m, |
| | yl)methyl) | | | 1H), 5.92 − 5.69 |
| | phosphonic acid | | | (m, 1H), 4.74 − |
| | | | | 4.51 (m, 2H), |
| | | | | 4.45 − 4.40 (m, |
| | | | | 1H), 4.33 − 4.26 |
| | | | | (m, 1H), 4.25 − |
| | | | | 4.14 (m, 2H), |
| | | | | 3.99 (d, J = 1.2 |
| | | | | Hz, 4H), 3.94 |
| | | | | (d, J = 4.8 Hz, |
| | | | | 3H), 2.23 − 2.15 |
| | | | | (m, 1H), 2.08 − |
| | | | | 1.92 (m, 2H), |
| | | | | 1.90 − 1.63 (m, 7H) |
| 207 | (fluoro(2- (((3S,6S,8R,9aR)- 3-(3-(4- fluoropyridin-3- yl)azetidine-1- carbonyl)-8- methyl-5- oxooctahydro-1H- pyrrolo[1,2- a]azepin-6- | 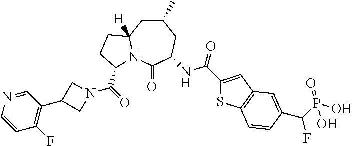 [SMILES: C[C@H]1C[C@H]2CC[C@@H](C(=O)N3CC(c4cnccc4F)C3)N2C(=O)[C@@H](NC(=O)c2cc3cc(C(F)P(=O)(O)O)ccc3s2)C1] | 633.0 | ¹H NMR (400 MHz, CD₃OD) δ 8.86 − 8.64 (m, 1H), 8.56 (s, 1H), 8.08 − 7.93 (m, 2H), 7.92 − 7.85 (m, 1H), 7.63 − 7.47 (m, 2H), 5.90 − 5.70 (m, 1H), 4.82 − |
| | yl)carbamoyl)benzo | | | 4.64 (m, 2H), |
| | [b]thiophen-5- | | | 4.60 − 4.51 (m, |
| | yl)methyl) | | | 1H), 4.47 − 3.98 |
| | phosphonic acid | | | (m, 5H), 2.37 − |
| | | | | 2.26 (m, 1H), |
| | | | | 2.20 − 1.91 (m, |
| | | | | 5H), 1.79 (m, |
| | | | | 1H), 1.70 − 1.52 |
| | | | | (m, 2H), 1.05 (d, |
| | | | | J = 6.4 Hz, 3H) |
| 208 | ((2-(((3S,6S,9aS)- 5-oxo-3-(3- (pyridin-3- yl)azetidine-1- carbonyl)octahydro- 1H-pyrrolo[1,2- a]azepin-6- yl)carbamoyl)thieno | 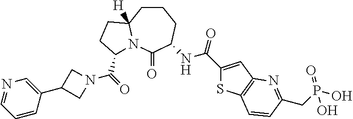 [SMILES: O=C(N[C@H]1CCC[C@H]2CC[C@@H](C(=O)N3CC(c4cccnc4)C3)N2C1=O)c1cc2nc(CP(=O)(O)O)ccc2s1] | 584.1 | ¹H NMR (400 MHz, CD₃OD) δ 8.59 − 8.50 (m, 1H), 8.46 − 8.42 (m, 1H), 8.28 (d, J = 8.4 Hz, 1H), 8.17 − 7.91 (m, 2H), 7.64 − 7.61 |
| | [3,2-b]pyridin-5- | | | (m, 1H), 7.49 − |
| | yl)methyl) | | | 7.43 (m, 1H), |
| | phosphonic acid | | | 4.78 − 4.74 (m, |
| | | | | 1H), 4.70 (t, J = |
| | | | | 8.8 Hz, 1H), |
| | | | | 4.63 − 4.55 (m, |
| | | | | 2H), 4.44 − 4.28 |
| | | | | (m, 1H), 4.14 − |
| | | | | 3.93 (m, 3H), |
| | | | | 3.40 − 3.33 (m, |
| | | | | 2H), 2.38 − 2.27 |
| | | | | (m, 1H), 2.19 − |
| | | | | 2.07 (m, 2H), |
| | | | | 2.06 − 1.95 (m, |
| | | | | 3H), 1.94 − 1.79 |
| | | | | (m, 4H) |
| 209 | (fluoro(2- (((3S,6S,8S,9aR)-3- (3-(4-fluoropyridin- 3-yl)azetidine-1- carbonyl)-8- methyl-5- oxooctahydro-1H- pyrrolo[1,2- a]azepin-6- yl)carbamoyl)benzo | 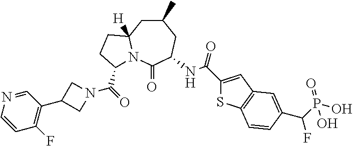 [SMILES: C[C@@H]1C[C@H]2CC[C@@H](C(=O)N3CC(c4cnccc4F)C3)N2C(=O)[C@@H](NC(=O)c2cc3cc(C(F)P(=O)(O)O)ccc3s2)C1] | 633.1 | ¹H NMR (400 MHz, CD₃OD) δ 8.85 − 8.64 (m, 1H), 8.61 − 8.51 (m, 1H), 8.10 − 7.83 (m, 3H), 7.63 − 7.53 (m, 1H), 7.48 (m, 1H), 5.88 − 5.70 (m, 1H), 5.04 − |
| | [b]thiophen-5- | | | 4.95 (m, 1H), |
| | yl)methyl) | | | 4.92 (s, 1H), |
| | phosphonic acid | | | 4.75 − 4.66 (m, |
| | | | | 1H), 4.54 − 4.40 |
| | | | | (m, 2H), 4.27 |
| | | | | (m, 1H), 4.23 − |
| | | | | 4.08 (m, 2H), |
| | | | | 2.39 − 2.24 (m, |
| | | | | 2H), 2.21 − 2.10 |
| | | | | (m, 1H), 2.09 − |
| | | | | 1.91 (m, 4H), |
| | | | | 1.87 − 1.78 (m, |
| | | | | 1H), 1.61 (m, |
| | | | | 1H), 1.28 − 1.16 |
| | | | | (m, 3H) |
| 210 | ((2- (((3S,6S,9R,10aR)- 9-ethyl-3-(3-(4- methoxypyridin-3- yl)azetidine-1- carbonyl)-5- oxodecahydropyrrolo [1,2-a]azocin-6- yl)carbamoyl)benzo [b]thiophen-5- yl)fluoromethyl) phosphonic acid | 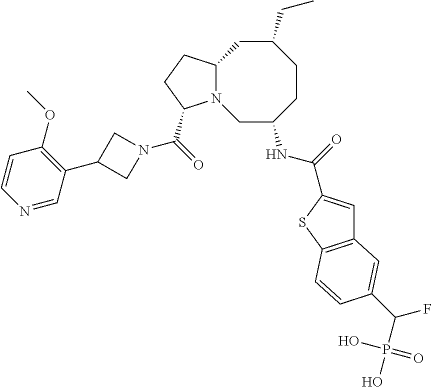 [SMILES: CC[C@@H]1CC[C@H](NC(=O)c2cc3cc(C(F)P(=O)(O)O)ccc3s2)CN2[C@H](CC[C@H]2C(=O)N2CC(c3cnccc3OC)C2)C1] | 673.1 | ¹H NMR (400 MHz, CD₃OD) δ 8.69 − 8.21 (m, 2H), 8.10 − 7.73 (m, 3H), 7.63 − 7.52 (m, 1H), 7.33 − 7.17 (m, 1H), 5.78 − 5.54 (m, 1H), 5.09 − 4.93 (m, 1H), 4.65 − 4.62 (m, 1H), 4.48 − 4.38 (m, 2H), 4.35 − 4.28 (m, 1H), 4.20 − 3.92 (m, 6H), 2.31 − 2.21 (m, 2H), 2.07 − 1.85 (m, 6H), 1.67 − 1.50 (m, 3H), 1.43 − 1.33 (m, 2H), 0.99 − 0.94 (m, 3H) |
| 211 | (fluoro(2- (((3S,6S,9R,10aR)- 9-isopropyl-3-(3- (4-methoxypyridin- 3-yl)azetidine-1- carbonyl)-5- oxodecahydropyrrolo [1,2-a]azocin-6- yl)carbamoyl)benzo [b]thiophen-5- yl)methyl) phosphonic acid | 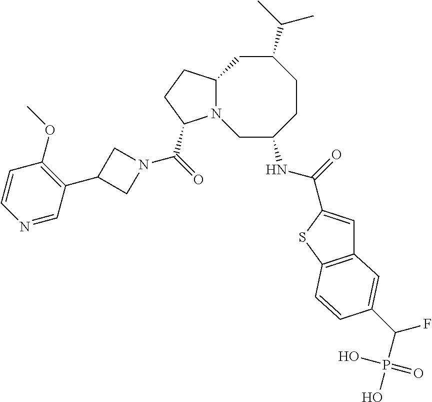 [SMILES: COc1ccncc1C1CN(C(=O)[C@@H]2CC[C@@H]3C[C@H](C(C)C)CC[C@H](NC(=O)c4cc5cc(C(F)P(=O)(O)O)ccc5s4)CN32)C1] | 687.1 | ¹H NMR (400 MHz, CD₃OD) δ 8.40 − 8.27 (m, 2H), 8.13 − 7.97 (m, 2H), 7.88 − 7.83 (m, 1H), 7.68 − 7.58 (m, 1H), 7.11 − 6.98 (m, 1H), 5.73 − 5.55 (m, 1H), 5.08 − 5.01 (m, 1H), 4.63 − 4.59 (m, 1H), 4.53 − 4.37 (m, 4H), 4.34 − 4.19 (m, 1H), 4.13 − 4.04 (m, 1H), 3.94 − 3.91 (m, 3H), 2.34 − 2.20 (m, 2H), 2.08 − 1.83 (m, 6H), 1.71 − 1.51 (m, 4H), 0.95 − 0.87 (m, 6H) |
| 212 | ((2-(((3S,6S,9R,10aR)- 3-((3R,4S)-3-cyano-4- phenylpyrrolidine- 1-carbonyl)-9- isopropyl-5- oxodecahydropyrrolo [1,2-a]azocin-6- yl)carbamoyl)benzo [b]thiophen-5- yl)fluoromethyl) phosphonic acid Or ((2-(((3S,6S,9R,10aR)- 3-((3S,4R)-3- cyano-4- phenylpyrrolidine- 1-carbonyl)-9- isopropyl-5- oxodecahydropyrrolo [1,2-a]azocin-6- yl)carbamoyl)benzo [b]thiophen-5- yl)fluoromethyl) phosphonic acid | 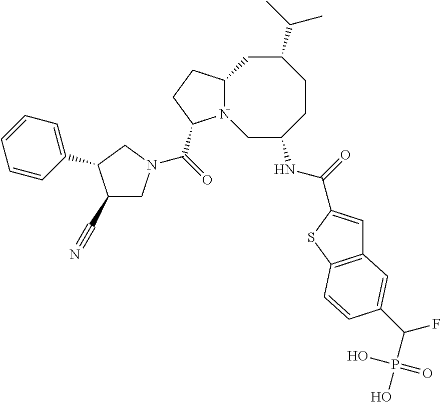 [SMILES: CC(C)[C@@H]1CC[C@H](NC(=O)c2cc3cc(C(F)P(=O)(O)O)ccc3s2)CN2[C@H](CC[C@H]2C(=O)N2C[C@@H](C#N)[C@H](c3ccccc3)C2)C1] | 695.1 | ¹H NMR (400 MHz, CD₃OD) δ 8.11 (d, J = 2.7 Hz, 1H), 8.02 (br. s., 1H), 7.93 (t, J = 7.9 Hz, 1H), 7.59 − 7.57 (m, 1H), 7.43 − 7.29 (m, 5H), 5.92 − 5.71 (m, 1H), 5.10 − 5.00 (m, 1H), 4.75 − 4.64 (m, 1H), 4.57 − 4.39 (m, 2H), 4.11 − 3.92 (m, 1H), 3.88 − 3.47 (m, 4H), 2.41 − 2.21 (m, 2H), 2.10 − 1.84 (m, 6H), 1.75 − 1.52 (m, 4H), 0.95 − 0.89 (m, 6H) |
| | | Or | | |
| | | 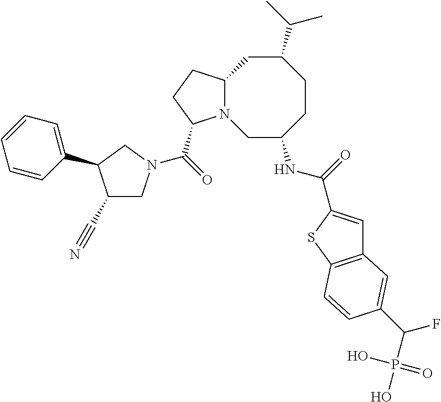 [SMILES: CC(C)[C@@H]1CC[C@H](NC(=O)c2cc3cc(C(F)P(=O)(O)O)ccc3s2)CN2[C@H](CC[C@H]2C(=O)N2C[C@H](c3ccccc3)[C@@H](C#N)C2)C1] | | |
| | | Pyrrolidine building block SFC | | |
| | | peak 2 was used for synthesis | | |
| | | and biological testing | | |
| 213 | ((2-(((3S,6S,9aS)- 3-(3-(4-((S)-2- (methoxymethyl) azetidin-1-yl)pyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 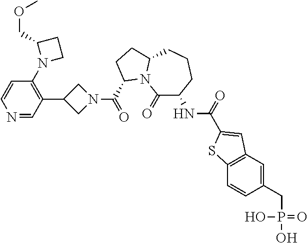 [SMILES: COC[C@@H]1CCN1c1ccncc1C1CN(C(=O)[C@@H]2CC[C@@H]3CCC[C@H](NC(=O)c4cc5cc(CP(=O)(O)O)ccc5s4)C(=O)N32)C1] | 682.1 | ¹H NMR (400 MHz, CD₃OD) δ 8.35 − 7.32 (m, 6H), 6.85 − 6.50 (m, 1H), 4.81 − 4.40 (m, 6H), 4.40 − 4.25 (m, 1H), 4.22 − 4.10 (m, 1H), 4.10 − 3.85 (m, 3H), 3.76 − 3.56 (m, 2H), 3.39 − 3.32 (m, 3H), 3.18 − 3.02 (m, 2H), 2.62 − 2.50 (m, |
| | | | | 1H), 2.40 − 1.72 |
| | | | | (m, 11H). |
| 214 | ((2-(((3S,6S,9aS)- 3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 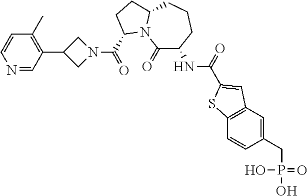 [SMILES: Cc1ccncc1C1CN(C(=O)[C@@H]2CC[C@@H]3CCC[C@H](NC(=O)c4cc5cc(CP(=O)(O)O)ccc5s4)C(=O)N32)C1] | 597.1 | ¹H NMR (400 MHz, CD₃OD) δ 8.57 (d, J = 17.1 Hz, 1H), 8.36 − 8.31 (m, 1H), 8.00 (d, J = 11.2 Hz, 1H), 7.93 (d, J = 11.2 Hz, 1H), 7.76 − 7.71 (m, 1H), 7.61 − 7.56 (m, 1H), 7.32 − 7.26 (m, 1H), 5.02 − |
| | | | | 4.95 (m, 1H), |
| | | | | 4.79 − 4.69 (m, |
| | | | | 2H), 4.66 − 4.52 |
| | | | | (m, 1H), 4.48 − |
| | | | | 4.39 (m, 1H), |
| | | | | 4.32 − 4.05 (m, |
| | | | | 3H), 3.02 (dd, |
| | | | | J = 19.6, 3.2 Hz, |
| | | | | 2H), 2.34 (s, |
| | | | | 4H), 2.24 − 1.74 |
| | | | | (m, 9H) |
| 215 | (difluoro(7- (((3′S,6′S,10a′S)-3′- (3-methoxy-3-(1- methyl-1H- imidazol-2- yl)azetidine-1- carbonyl)-5′- oxooctahydro-5′H- spiro[cyclopropane- 1,8′-pyrrolo[1,2- a]azocin]-6′- | 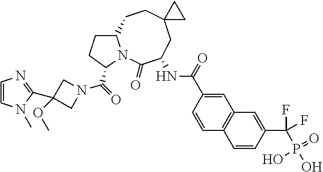 | 686.1 | ¹H NMR (400 MHz, DMSO- d₆) δ 8.64 − 8.58 (m, 1H), 8.57 − 8.50 (m, 1H), 8.14 (d, J = 13.7 Hz, 1H), 8.05 − 7.90 (m, 3H), 7.77 − 7.69 (m, 1H), 7.22 (d, J = 13.7 Hz, 1H), |
| | yl)carbamoyl) | | | 6.85 (d, J = 5.4 |
| | naphthalen-2- | | | Hz, 1H), 5.04 − |
| | yl)methyl) | | | 4.89 (m, 1.5H), |
| | phosphonic acid | | | 4.72 (q, J = 9.5 |
| | | | | Hz, 1H), 4.50 − |
| | | | | 4.30 (m, 3.5H), |
| | | | | 4.11 − 3.98 (m, |
| | | | | 1H), 3.55 (d, J = |
| | | | | 7.6 Hz, 3H), |
| | | | | 2.90 (d, J = 11.0 |
| | | | | Hz, 3H), 2.26 − |
| | | | | 2.16 (m, 0.5H), |
| | | | | 2.15 − 2.02 (m, |
| | | | | 1.5H), 1.98 − |
| | | | | 1.80 (m, 3H), |
| | | | | 1.80 − 1.67 (m, |
| | | | | 2H), 1.64 − 1.46 |
| | | | | (m, 3H), 0.53 − |
| | | | | 0.28 (m, 4H) |
| 216 | ((2- (((3′S,6′S,10a′S)-5′- oxo-3′-(3-(pyridin- 3-yl)azetidine-1- carbonyl)octahydro- 5′H- spiro[cyclopropane- 1,8′-pyrrolo[1,2- a]azocin]-6′- yl)carbamoyl)benzo [b]thiophen-5- yl)methyl) phosphonic acid | 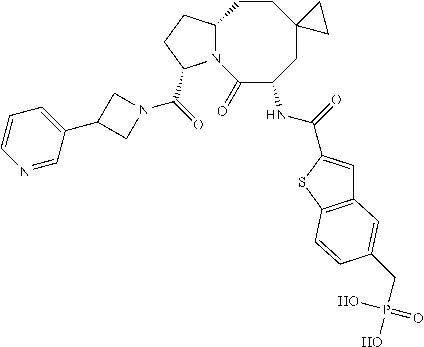 [SMILES: O=C(N[C@H]1CC2(CC[C@H]3CC[C@@H](C(=O)N4CC(c5cccnc5)C4)N3C1=O)CC2)c1cc2cc(CP(=O)(O)O)ccc2s1] | 623.1 | ¹H NMR (400 MHz, DMSO- d₆) δ 8.70 − 8.59 (m, 1H), 8.58 − 8.53 (m, 1H), 8.50 − 8.44 (m, 1H), 8.31 (s, 1H), 8.12 (d, J = 6.8 Hz, 1H), 7.88 − 7.75 (m, 2H), 7.72 − 7.66 (m, 1H), 7.40 (dt, J = 7.9, 4.2 Hz, 1H), 7.33 − 7.26 (m, 1H), 4.98 − 4.84 (m, 1.5H), 4.66 − 4.57 (m, 0.5H), 4.56 − 4.48 (m, 0.5H), 4.42 − 4.18 (m, 4H), 3.99 − 3.83 (m, |
| | | | | 2.5H), 2.86 − |
| | | | | 2.75 (m, 2H), |
| | | | | 2.22 − 2.00 (m, |
| | | | | 2H), 1.99 − 1.66 |
| | | | | (m, 5H), 1.60 − |
| | | | | 1.44 (m, 3H), |
| | | | | 0.54 − 0.32 (m, 4H) |
| 217 | ((7-(((3S,6S,10aS)- 5-oxo-3-(3- (pyridin-3- yl)pyrrolidine-1- carbonyl)decahydro pyrrolo[1,2- a]azocin-6- yl)carbamoyl) naphthalen-2- yl)methyl) phosphonic acid | 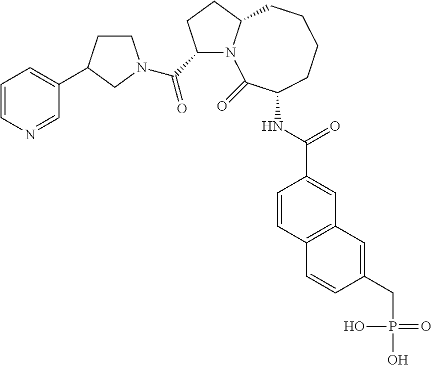 [SMILES: O=C(N[C@H]1CCCC[C@H]2CC[C@@H](C(=O)N3CCC(c4cccnc4)C3)N2C1=O)c1ccc2ccc(CP(=O)(O)O)cc2c1] | 605.2 | ¹H NMR (400 MHz, DMSO- d₆) δ 8.66 − 8.50 (m, 2H), 8.48 − 8.36 (m, 2H), 7.98 − 7.79 (m, 4H), 7.77 − 7.69 (m, 1H), 7.57 − 7.48 (m, 1H), 7.40 − 7.25 (m, 1H), 5.09 − 4.92 (m, 1H), 4.64 − 4.49 (m, 1H), 4.39 − 4.20 (m, 1H), 4.06 − 3.89 (m, 1H), 3.87 − 3.71 (m, 1H), 3.65 − 3.47 (m, 2H), 3.15 − 3.06 (m, 2H), 2.32 − 2.17 (m, 2H), 2.14 − 1.69 (m, 10H), 1.66 − |
| | | | | 1.47 (m, 3H) |
| 218 | (difluoro(7- (((3S,6S,10aS)-5- oxo-3-(3-(pyridin- 3-yl)pyrrolidine-1- carbonyl)decahydro pyrrolo[1,2- a]azocin-6- yl)carbamoyl) naphthalen-2- yl)methyl) phosphonic acid | 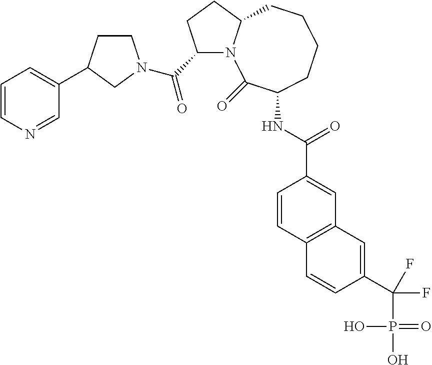 [SMILES: O=C(N[C@H]1CCCC[C@H]2CC[C@@H](C(=O)N3CCC(c4cccnc4)C3)N2C1=O)c1ccc2ccc(C(F)(F)P(=O)(O)O)cc2c1] | 641.1 | ¹H NMR (400 MHz, CD₃OD) δ 8.70 − 8.15 (m, 5H), 7.99 − 7.74 (m, 5H), 5.19 − 5.06 (m, 1H), 4.76 − 4.66 (m, 1H), 4.55 − 4.43 (m, 1H), 4.13 − 4.00 (m, 1H), 3.98 − 3.65 (m, 3H), 2.40 − 2.23 (m, 3H), 2.13 − 1.96 (m, 7H), 1.94 − 1.78 (m, 3H), 1.71 (br. s., 2H) |
| 219 | ((2-(((3S,6S,9aS)- 3-(((3S,4S)-1- acetyl-4- fluoropyrrolidin-3- yl)carbamoyl)-5- oxooctahydro-1H- pyrrolo[1,2- a]azepin-6- yl)carbamoyl)benzo [b]thiophen-5- yl)methyl) phosphonic acid | 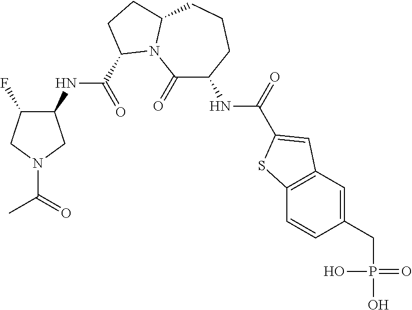 [SMILES: CC(=O)N1C[C@H](NC(=O)[C@@H]2CC[C@@H]3CCC[C@H](NC(=O)c4cc5cc(CP(=O)(O)O)ccc5s4)C(=O)N32)[C@@H](F)C1] | 595.1 | ¹H NMR (400 MHz, CD₃OD) δ 8.49 (d, J = 6.6 Hz, 1H), 8.40 (d, J = 5.9 Hz, 1H), 8.00 (d, J = 4.4 Hz, 1H), 7.89 − 7.78 (m, 2H), 7.43 (d, J = 8.6 Hz, 1H), 5.18 − 4.95 (m, 1H), 4.72 (d, J = 10.8 Hz, 1H), 4.54 − 4.35 (m, 2H), 4.11 − 3.99 (m, 1H), 3.95 − 3.80 (m, 1H), 3.80 − 3.60 (m, 3H), 3.28 − 3.20 |
| | | | | (m, 2H), 2.35 − |
| | | | | 2.22 (m, 1H), 2.18 − |
| | | | | 1.75 (m, 12H) |
| 220 | ((2-(((3S,6S,9aS)- 3-(((3S,4S)-1- acetyl-4- fluoropyrrolidin-3- yl)carbamoyl)-5- oxooctahydro-1H- pyrrolo[1,2- a]azepin-6- yl)carbamoyl)benzo [b]thiophen-5- yl)methyl) phosphonic acid | 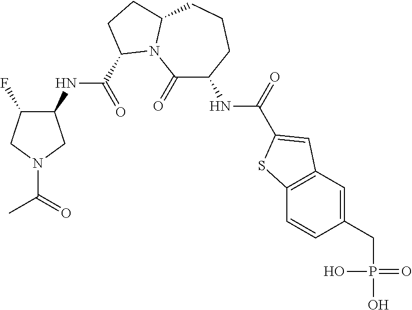 [SMILES: CC(=O)N1C[C@H](NC(=O)[C@@H]2CC[C@@H]3CCC[C@H](NC(=O)c4cc5cc(CP(=O)(O)O)ccc5s4)C(=O)N32)[C@@H](F)C1] | 595.1 | ¹H NMR (400 MHz, CD₃OD) δ 8.51 − 8.44 (m, 1H), 8.02 − 7.97 (m, 1H), 7.88 − 7.80 (m, 2H), 7.46 − 7.38 (m, 1H), 5.28 − 5.03 (m, 1H), 4.73 (d, J = 11.0 Hz, 1H), 4.67 − 4.43 (m, 2H), 4.10 − 4.00 (m, 1H), 4.00 − 3.92 (m, 1H), 3.92 − 3.80 (m, 1H), 3.79 − 3.53 (m, 1H), 3.41 (t, J = 10.3 Hz, 0.6H), 3.29 − |
| | | | | 3.17 (m, 2.4H), |
| | | | | 2.36 − 2.24 (m, |
| | | | | 1H), 2.20 − 1.72 |
| | | | | (m, 12H) |
| 221 | (1-fluoro-1-(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)ethyl)phosphonic acid | 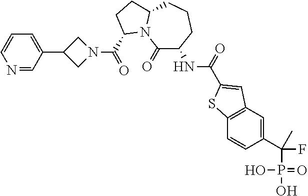 [SMILES: CC(F)(c1ccc2sc(C(=O)N[C@H]3CCC[C@H]4CC[C@@H](C(=O)N5CC(c6cccnc6)C5)N4C3=O)cc2c1)P(=O)(O)O] | 615.1 | ¹H NMR (400 MHz, CD₃OD) δ 8.58 − 8.41 (m, 2H), 8.14 − 7.93 (m, 3H), 7.85 − 7.83 (m, 1H), 7.72 − 7.70 (m, 1H), 7.50 − 7.44 (m, 1H), 4.99 − 4.94 (m, 1H), 4.72 − 4.68 (m, 1H), 4.62 − 4.53 (m, 2H), 4.46 − |
| | | | | 4.28 (m, 1H), |
| | | | | 4.11 − 3.97 (m, |
| | | | | 3H), 2.37 − 2.25 |
| | | | | (m, 1H), 2.20 − |
| | | | | 2.07 (m, 2H), |
| | | | | 2.05 − 1.79 |
| | | | | (m, 10H) |
| 222 | (1-fluoro-1-(2- (((3S,6S,10aS)-5- oxo-3-(3-(pyridin- 3-yl)pyrrolidine-1- carbonyl)decahydro pyrrolo[1,2- a]azocin-6- yl)carbamoyl)benzo [b]thiophen-5- yl)ethyl)phosphonic acid | 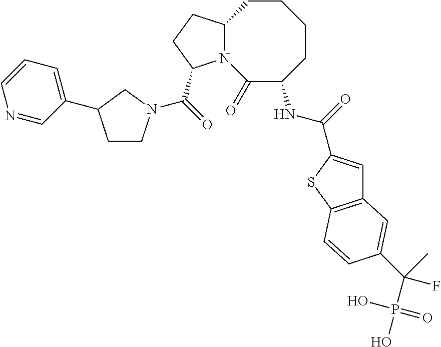 [SMILES: CC(F)(c1ccc2sc(C(=O)N[C@H]3CCCC[C@H]4CC[C@@H](C(=O)N5CCC(c6cccnc6)C5)N4C3=O)cc2c1)P(=O)(O)O] | 643.1 | ¹H NMR (400 MHz, CD₃OD) δ 8.54 − 8.48 (m, 1H), 8.45 − 8.37 (m, 1H), 8.14 − 8.05 (m, 2H), 7.91 − 7.73 (m, 3H), 7.47 − 7.37 (m, 1H), 5.09 − 5.00 (m, 1H), 4.76 − 4.66 (m, 1H), 4.48 − 4.36 (m, 1H), 4.18 − 3.75 (m, 2H), 3.74 − 3.34 (m, 3H), 2.51 − 2.20 (m, 3H), 2.20 − 1.76 (m, 12H), 1.75 − 1.59 (m, 2H) |
| 223 | (fluoro(2- (((3S,6S,10aS)-5- oxo-3-(3-(pyridin- 3-yl)pyrrolidine-1- carbonyl)decahydro pyrrolo[1,2- a]azocin-6- yl)carbamoyl)benzo [b]thiophen-5- yl)methyl)phospho nic acid | 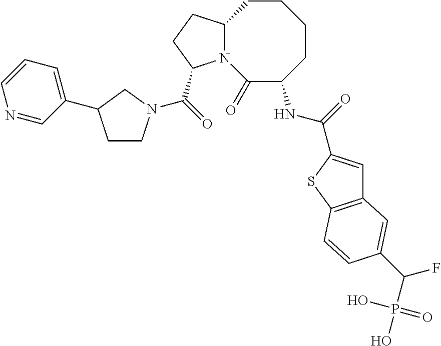 [SMILES: O=C(N[C@H]1CCCC[C@H]2CC[C@@H](C(=O)N3CCC(c4cccnc4)C3)N2C1=O)c1cc2cc(C(F)P(=O)(O)O)ccc2s1] | 629.1 | ¹H NMR (400 MHz, CD₃OD) δ 8.56 − 8.47 (m, 1H), 8.44 − 8.36 (m, 1H), 8.12 − 8.02 (m, 2H), 7.90 − 7.78 (m, 2H), 7.68 − 7.66 (m, 1H), 7.47 − 7.37 (m, 1H), 5.72 − 5.52 (m, 1H), 5.09 − 5.03 (m, 1H), 4.75 − 4.72 (m, 1H), 4.47 − 4.38 (m, 1H), 4.18 − 3.77 (m, 2H), 3.74 − 3.34 (m, 3H), 2.49 − 2.21 (m, 3H), 2.20 − 1.92 (m, 7H), 1.91 − 1.58 (m, 4H) |
| 224 | ((2-(((3S,6S,10aS)- 3-((3S,4R)-3- cyano-4- phenylpyrrolidine- 1-carbonyl)-5- oxodecahydropyrrolo [1,2-a]azocin-6- yl)carbamoyl)benzo [b]thiophen-5- yl)fluoromethyl) phosphonic acid Or ((2-(((3S,6S,10aS)- 3-((3R,4S)-3- cyano-4- phenylpyrrolidine- 1-carbonyl)-5- oxodecahydropyrrolo [1,2-a]azocin-6- yl)carbamoyl)benzo [b]thiophen-5- yl)fluoromethyl) phosphonic acid | 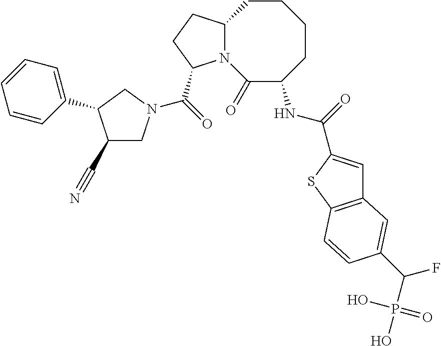 [SMILES: N#C[C@@H]1CN(C(=O)[C@@H]2CC[C@@H]3CCCC[C@H](NC(=O)c4cc5cc(C(F)P(=O)(O)O)ccc5s4)C(=O)N32)C[C@H]1c1ccccc1] Or | 653.1 | ¹H NMR (400 MHz, CD₃OD) δ 8.11 (s, 1H), 8.03 (br. s., 1H), 7.96 − 7.88 (m, 1H), 7.61 − 7.59 (m, 1H), 7.43 − 7.29 (m, 5H), 5.87 − 5.68 (m, 1H), 5.07 − 4.98 (m, 1H), 4.74 − 4.64 (m, 1H), 4.57 − 4.38 (m, 2H), 4.10 − 3.92 (m, 1H), 3.88 − 3.67 (m, 2H), 3.66 − 3.48 (m, 2H), 2.40 − 2.17 (m, 2H), 2.11 − 1.93 (m, 6H), 1.91 − 1.76 (m, 2H), 1.74 − 1.57 (m, 2H) |
| | | 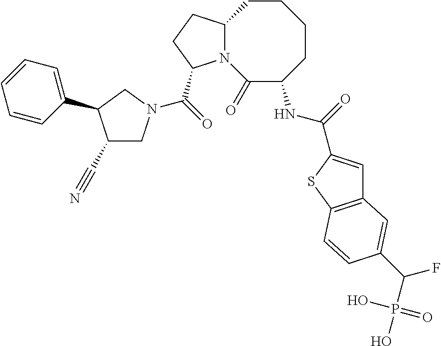 [SMILES: N#C[C@H]1CN(C(=O)[C@@H]2CC[C@@H]3CCCC[C@H](NC(=O)c4cc5cc(C(F)P(=O)(O)O)ccc5s4)C(=O)N32)C[C@@H]1c1ccccc1] | | |
| | | Pyrrolidine building block SFC | | |
| | | peak 2 was used for synthesis | | |
| | | and biological testing | | |
| 225 | ((2-(((3S,6S,10aS)- 3-((3S,4R)-3-cyano- 4-phenylpyrrolidine- 1-carbonyl)-5- oxodecahydropyrrolo [1,2-a]azocin-6- yl)carbamoyl)benzo [b]thiophen-5- yl)fluoromethyl) phosphonic acid Or ((2-(((3S,6S,10aS)- 3-((3R,4S)-3-cyano-4- phenylpyrrolidine- 1-carbonyl)-5- oxodecahydropyrrolo [1,2-a]azocin-6-yl) carbamoyl)benzo[b] thiophen-5- yl)fluoromethyl) phosphonic acid | 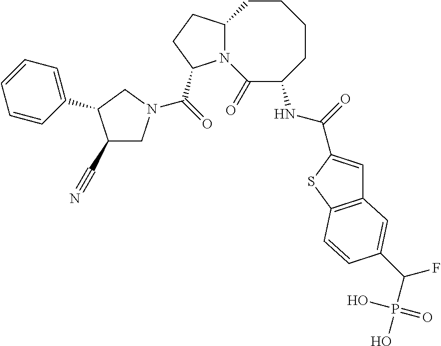 [SMILES: N#C[C@@H]1CN(C(=O)[C@@H]2CC[C@@H]3CCCC[C@H](NC(=O)c4cc5cc(C(F)P(=O)(O)O)ccc5s4)C(=O)N32)C[C@H]1c1ccccc1] | 653.1 | ¹H NMR (400 MHz, CD₃OD) δ 8.15 − 8.07 (m, 1H), 8.03 (br. s., 1H), 7.98 − 7.90 (m, 1H), 7.60 − 7.58 (m, 1H), 7.46 − 7.28 (m, 5H), 5.90 − 5.73 (m, 1H), 5.07 − 4.99 (m, 1H), 4.68 − 4.59 (m, 1H), 4.48 − 4.39 (m, 1H), 4.22 − 4.17 (m, 1H), 4.15 − 4.06 (m, 1H), 3.96 − 3.73 (m, 1H), 3.69 − 3.37 (m, 3H), 2.41 − 2.19 (m, 2H), 2.12 − 1.93 (m, 6H), 1.92 − 1.76 (m, 2H), 1.74 − 1.57 (m, 2H) |
| | | Or | | |
| | | 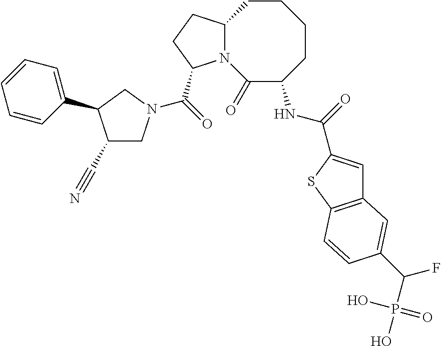 [SMILES: N#C[C@H]1CN(C(=O)[C@@H]2CC[C@@H]3CCCC[C@H](NC(=O)c4cc5cc(C(F)P(=O)(O)O)ccc5s4)C(=O)N32)C[C@@H]1c1ccccc1] | | |
| | | Pyrrolidine building block SFC | | |
| | | peak 1 was used for synthesis | | |
| | | and biological testing | | |
| 226 | ((7-(((3S,6S,10aS)- 3-((3R,4S)-3- cyano-4- phenylpyrrolidine- 1-carbonyl)-5- oxodecahydropyrrolo [1,2-a]azocin-6- yl)carbamoyl) naphthalen-2- yl)fluoromethyl) phosphonic acid Or ((7-(((3S,6S,10aS)- 3-((3S,4R)-3-cyano- 4-phenylpyrrolidine- 1-carbonyl)-5- oxodecahydropyrrolo [1,2-a]azocin-6- yl)carbamoyl) naphthalen-2- yl)fluoromethyl) phosphonic acid | 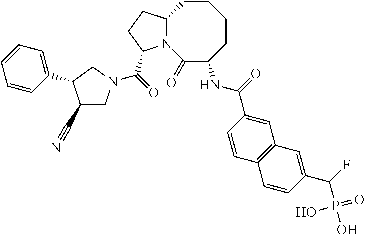 [SMILES: N#C[C@@H]1CN(C(=O)[C@@H]2CC[C@@H]3CCCC[C@H](NC(=O)c4ccc5ccc(C(F)P(=O)(O)O)cc5c4)C(=O)N32)C[C@H]1c1ccccc1] Or 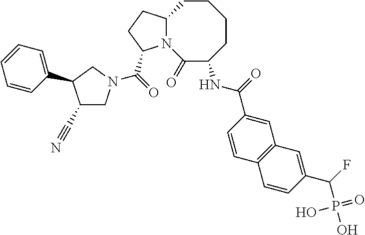 [SMILES: N#C[C@H]1CN(C(=O)[C@@H]2CC[C@@H]3CCCC[C@H](NC(=O)c4ccc5ccc(C(F)P(=O)(O)O)cc5c4)C(=O)N32)C[C@@H]1c1ccccc1] | 647.1 | ¹H NMR (400 MHz, CD₃OD) δ 8.48 (d, J = 4.4 Hz, 1H), 8.12 − 8.05 (m, 1H), 8.00 − 7.91 (m, 3H), 7.77 − 7.69 (m, 1H), 7.44 − 7.29 (m, 5H), 5.99 − 5.80 (m, 1H), 5.15 − 5.04 (m, 1H), 4.76 − 4.64 (m, 1H), 4.59 − 4.41 (m, 2H), 4.07 (dd, J = 11.5, 8.1 Hz, 0.5H), 3.96 (dd, J = 11.7, 7.8 Hz, 0.5H), 3.85 (t, J = 9.5 Hz, 0.5H), 3.80 − 3.50 (m, 3.5H), 2.42 − 2.18 (m, 2H), 2.13 − 1.94 (m, 6H), 1.93 − 1.76 (m, 2H), 1.76 − 1.59 (m, 2H) |
| | | Pyrrolidine building block SFC | | |
| | | peak 2 was used for synthesis | | |
| | | and biological testing | | |
| 227 | ((2-(((3S,6S,10aS)- 3-((3S,4R)-3- cyano-4-(1-methyl- 2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)fluoromethyl) phosphonic acid Or ((2-(((3S,6S,10aS)- 3-((3R.4S)-3- cyano-4-(1-methyl- 2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)fluoromethyl) phosphonic acid | 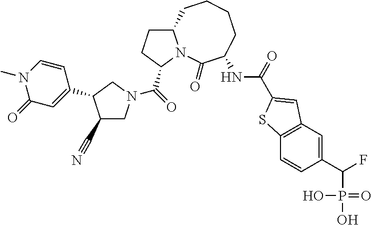 [SMILES: Cn1ccc([C@@H]2CN(C(=O)[C@@H]3CC[C@@H]4CCCC[C@H](NC(=O)c5cc6cc(C(F)P(=O)(O)O)ccc6s5)C(=O)N43)C[C@H]2C#N)cc1=O] Or 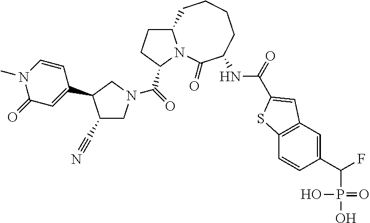 [SMILES: Cn1ccc([C@H]2CN(C(=O)[C@@H]3CC[C@@H]4CCCC[C@H](NC(=O)c5cc6cc(C(F)P(=O)(O)O)ccc6s5)C(=O)N43)C[C@@H]2C#N)cc1=O] | 684.1 | ¹H NMR (400 MHz, CD₃OD) δ 8.11 − 8.09 (m, 1H), 8.04 − 8.00 (m, 1H), 7.96 − 7.89 (m, 1H), 7.70 − 7.54 (m, 2H), 6.64 − 6.56 (m, 1H), 6.48 − 6.43 (m, 1H), 5.89 − 5.71 (m, 1H), 5.07 − 4.97 (m, 1H), 4.66 − 4.57 (m, 1H), 4.49 − 4.39 (m, 1H), 4.22 − 4.06 (m, 2H), 3.92 − 3.35 (m, 7H), 2.40 − 2.18 (m, 2H), 2.10 − 1.92 (m, 6H), 1.90 − 1.75 (m, 2H), 1.74 − 1.57 (m, 2H) |
| | | Pyrrolidine building block SFC | | |
| | | peak 2 was used for synthesis | | |
| | | and biological testing | | |
| 228 | ((2-(((3S,6S,10aS)- 3-((3S,4R)-3- cyano-4-(1-methyl- 2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)fluoromethyl) phosphonic acid Or ((2-(((3S,6S,10aS)- 3-((3R.4S)-3- cyano-4-(1-methyl- 2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)fluoromethyl) phosphonic acid | 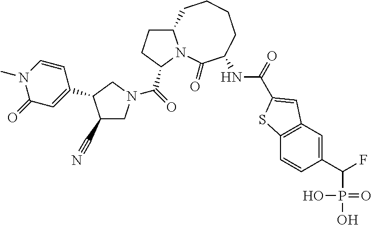 [SMILES: Cn1ccc([C@@H]2CN(C(=O)[C@@H]3CC[C@@H]4CCCC[C@H](NC(=O)c5cc6cc(C(F)P(=O)(O)O)ccc6s5)C(=O)N43)C[C@H]2C#N)cc1=O] Or 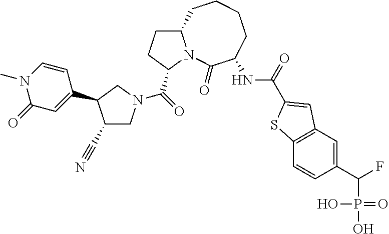 [SMILES: Cn1ccc([C@H]2CN(C(=O)[C@@H]3CC[C@@H]4CCCC[C@H](NC(=O)c5cc6cc(C(F)P(=O)(O)O)ccc6s5)C(=O)N43)C[C@@H]2C#N)cc1=O] | 684.1 | ¹H NMR (400 MHz, CD₃OD) δ 8.11 (s, 1H), 8.02 (br. s., 1H), 7.96 − 7.89 (m, 1H), 7.67 (t, J = 7.7 Hz, 1H), 7.60 − 7.58 (m, 1H), 6.58 (d, J = 11.7 Hz, 1H), 6.47 − 6.39 (m, 1H), 5.89 − 5.68 (m, 1H), 5.04 − 4.97 (m, 1H), 4.69 − 4.65 (m, 1H), 4.56 − 4.39 (m, 2H), 4.07 − 3.56 (m, 5H), 3.56- 3.54 (m, 3H), 2.39 − 2.19 (m, 2H), 2.12 − 1.94 (m, 6H), 1.91 − 1.76 (m, 2H), 1.74 − 1.57 (m, 2H) |
| | | Pyrrolidine building block SFC | | |
| | | peak 1 was used for synthesis | | |
| | | and biological testing | | |
| 229 | ((2-(((3S,6S,10aS)- 3-((rel-trans)-7- cyano-6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) phosphonic acid | 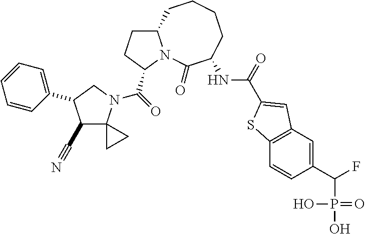 [SMILES: N#C[C@H]1[C@H](c2ccccc2)CN(C(=O)[C@@H]2CC[C@@H]3CCCC[C@H](NC(=O)c4cc5cc(C(F)P(=O)(O)O)ccc5s4)C(=O)N32)C12CC2] | 679.1 | ¹H NMR (400 MHz, CD₃OD) δ 8.10 (s, 1H), 8.04 − 7.97 (m, 1H), 7.92 (d, J = 8.8 Hz, 1H), 7.59 (d, J = 7.8 Hz, 1H), 7.49 − 7.24 (m, 5H), 5.90 − 5.69 (m, 1H), 5.08 − 4.96 (m, 1H), 4.66 − 4.55 (m, 1H), 4.46 − 4.31 (m, 1H), 4.22 − 4.05 |
| | | | | (m, 1H), 3.85 − |
| | | | | 3.66 (m, 2H), |
| | | | | 2.42 − 2.12 (m, |
| | | | | 3H), 2.12 − 1.48 |
| | | | | (m, 12H), 1.04 − |
| | | | | 0.92 (m, 1H), |
| | | | | 0.78 − 0.62 (m, 1H) |
| 230 | ((2-(((3S,6S,10aS)- 3-((rel-trans)-3- cyano-4-(2- methoxypyridin-3- yl)pyrrolidine-1- carbonyl)-5- oxodecahydropyrrolo [1,2-a]azocin-6- yl)carbamoyl)benzo [b]thiophen-5- yl)fluoromethyl) phosphonic acid | 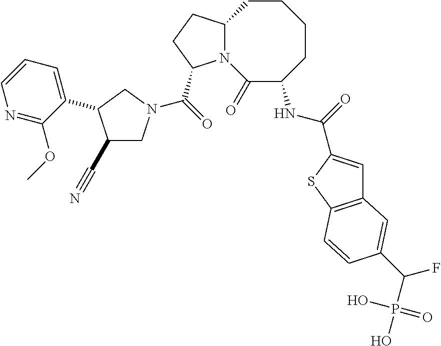 [SMILES: COc1ncccc1[C@@H]1CN(C(=O)[C@@H]2CC[C@@H]3CCCC[C@H](NC(=O)c4cc5cc(C(F)P(=O)(O)O)ccc5s4)C(=O)N32)C[C@H]1C#N] | 684.1 | ¹H NMR (400 MHz, CD₃OD) δ 8.16 − 8.06 (m, 2H), 8.05 − 7.99 (m, 1H), 7.97 − 7.88 (m, 1H), 7.82 − 7.75 (m, 0.3H), 7.73 − 7.65 (m, 0.7H), 7.63 − 7.55 (m, 1H), 7.03 − 6.94 (m, 1H), 5.87 − 5.69 (m, 1H), 5.09 − 4.98 (m, 1H), 4.72 − 4.61 (m, 1H), 4.49 − 4.36 (m, 1.5H), 4.13 − 3.58 (m, 8.5H), 2.41 − 2.18 (m, 2H), 2.12 − 1.93 (m, |
| | | | | 6H), 1.92 − 1.75 |
| | | | | (m, 2H), 1.74 − |
| | | | | 1.58 (m, 2H) |
| 231 | ((2-(((3S,6S,10aS)- 3-((rel-trans)-7- cyano-6-(3- methoxyphenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) phosphonic acid | 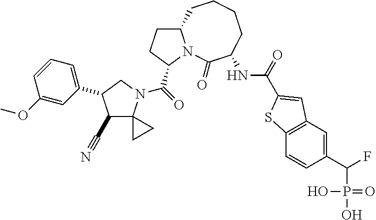 [SMILES: COc1cccc([C@@H]2CN(C(=O)[C@@H]3CC[C@@H]4CCCC[C@H](NC(=O)c5cc6cc(C(F)P(=O)(O)O)ccc6s5)C(=O)N43)C3(CC3)[C@H]2C#N)c1] | 709.1 | ¹H NMR (400 MHz, CD₃OD) δ 8.14 − 8.07 (m, 1H), 8.05 − 7.98 (m, 1H), 7.93 (d, J = 7.1 Hz, 1H), 7.59 (d, J = 8.6 Hz, 1H), 7.33 − 7.22 (m, 1H), 7.07 − 7.00 (m, 1H), 6.99 − 6.94 (m, 1H), 6.90 − 6.82 (m, 1H), 5.91 − 5.70 (m, |
| | | | | 1H), 5.05 − 4.96 |
| | | | | (m, 1H), 4.67 − |
| | | | | 4.55 (m, 1H), |
| | | | | 4.45 − 4.33 (m, |
| | | | | 1H), 4.20 − 4.06 |
| | | | | (m, 1H), 3.83 − |
| | | | | 3.68 (m, 5H), |
| | | | | 2.43 − 2.12 (m, |
| | | | | 3H), 2.08 − 1.73 |
| | | | | (m, 9H), 1.71 − |
| | | | | 1.53 (m, 2H), |
| | | | | 1.38 − 1.24 (m, |
| | | | | 1H), 1.02 − 0.93 |
| | | | | (m, 1H), 0.79 − |
| | | | | 0.65 (m, 1H) |
| 232 | ((2-(((3S,6S,9aS)- 3-((3S,4R)-3- cyano-4- phenylpyrrolidine- 1-carbonyl)-5- oxooctahydro-1H- pyrrolo[1,2- a]azepin-6- yl)carbamoyl)benzo [b]thiophen-5- yl)fluoromethyl) phosphonic acid Or ((2-(((3S,6S,9aS)- 3-((3R,4S)-3- cyano-4- phenylpyrrolidine- 1-carbonyl)-5- oxooctahydro-1H- pyrrolo[1,2- a]azepin-6- yl)carbamoyl)benzo [b]thiophen-5- yl)fluoromethyl) phosphonic acid | 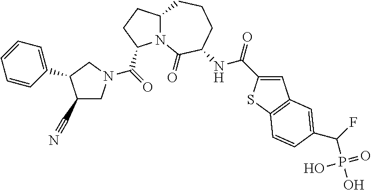 [SMILES: N#C[C@@H]1CN(C(=O)[C@@H]2CC[C@@H]3CCC[C@H](NC(=O)c4cc5cc(C(F)P(=O)(O)O)ccc5s4)C(=O)N32)C[C@H]1c1ccccc1] Or 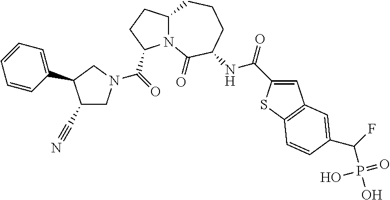 [SMILES: N#C[C@H]1CN(C(=O)[C@@H]2CC[C@@H]3CCC[C@H](NC(=O)c4cc5cc(C(F)P(=O)(O)O)ccc5s4)C(=O)N32)C[C@@H]1c1ccccc1] | 639.1 | ¹H NMR (400 MHz, CD₃OD) δ 8.08 (s, 1H), 8.04 (s, 1H), 7.98 − 7.92 (m , 1H), 7.63 − 7.57 (m, 1H), 7.46 − 7.30 (m, 5H), 5.90 − 5.73 (m, 1H), 4.83 − 4.72 (m, 2H), 4.53 − 4.33 (m, 1H), 4.14 − 3.95 (m, 2H), 3.89 − 3.49 (m, 4H), 2.39 − 2.25 (m, 1H), 2.25 − 2.06 (m, 2H), 2.06 − 1.97 (m, 2H), 1.97 − 1.78 (m, 5H) |
| | | Pyrrolidine building block SFC | | |
| | | peak 2 was used for synthesis | | |
| | | and biological testing | | |
| 233 | ((2- (((3S,6S,7S,10aS)- 7-cyclopropyl-5- oxo-3-(3-(pyridin- 3-yl)azetidine-1- carbonyl)decahydro pyrrolo[1,2- a]azocin-6- yl)carbamoyl)benzo [b]thiophen-5- yl)difluoromethyl) phosphonic acid | 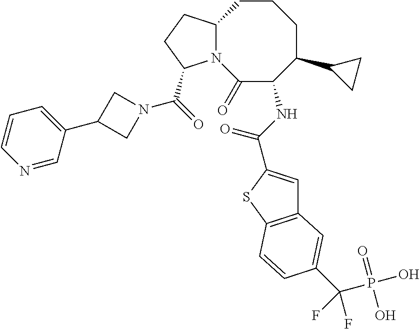 [SMILES: O=C(N[C@@H]1C(=O)N2[C@@H](CCC[C@H]1C1CC1)CC[C@H]2C(=O)N1CC(c2cccnc2)C1)c1cc2cc(C(F)(F)P(=O)(O)O)ccc2s1] | 673.1 | ¹H NMR (400 MHz, CD₃OD) δ 8.63 − 8.50 (m, 1H), 8.48 − 8.41 (m, 1H), 8.29 (s, 1H), 8.13 − 8.10 (m, 1H), 8.09 − 7.92 (m, 1H), 7.90 − 7.83 (m, 2H), 7.56 − 7.41 (m, 1H), 5.29 − 5.15 (m, 1H), 4.73 − 4.57 (m, 1H), 4.50 − 4.22 (m, 3H), 4.12 − 3.91 (m, 2H), 2.33 − 2.00 (m, 5H), 1.94 − 1.82 (m, 4H), 1.75 − 1.64 (m, 1H), |
| | | | | 1.28 − 1.17 (m, |
| | | | | 1H), 1.09 − 1.01 |
| | | | | (m, 1H), 0.98 − |
| | | | | 0.84 (m, 1H), |
| | | | | 0.67 − 0.51 (m, |
| | | | | 2H), 0.46 − 0.34 |
| | | | | (m, 1H), 0.09 − |
| | | | | −0.03 (m, 1H) |
| 234 | ((2-(((3S,6S,7S,10aS)- 3-((3R,4S)-3-cyano-4- phenylpyrrolidine- 1-carbonyl)-7- cyclopropyl-5- oxodecahydropyrrolo [1,2-a]azocin-6- yl)carbamoyl)benzo [b]thiophen-5- yl)difluoromethyl) phosphonic acid Or ((2-(((3S,6S,7S,10aS)- 3-((3S,4R)-3-cyano-4- phenylpyrrolidine- 1-carbonyl)-7- cyclopropyl-5- oxodecahydropyrrolo [1,2-a]azocin-6- yl)carbamoyl)benzo [b]thiophen-5- yl)difluoromethyl) phosphonic acid | 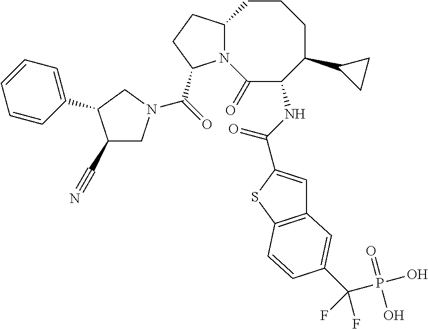 [SMILES: N#C[C@@H]1CN(C(=O)[C@@H]2CC[C@@H]3CCC[C@@H](C4CC4)[C@H](NC(=O)c4cc5cc(C(F)(F)P(=O)(O)O)ccc5s4)C(=O)N32)C[C@H]1c1ccccc1] Or 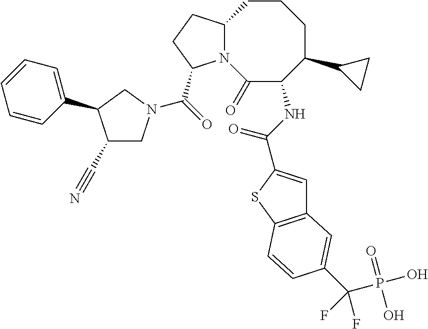 [SMILES: N#C[C@H]1CN(C(=O)[C@@H]2CC[C@@H]3CCC[C@@H](C4CC4)[C@H](NC(=O)c4cc5cc(C(F)(F)P(=O)(O)O)ccc5s4)C(=O)N32)C[C@@H]1c1ccccc1] | 711.1 | ¹H NMR (400 MHz, CD₃OD) δ 8.21 − 8.15 (m, 1H), 8.12 (s, 1H), 8.06 − 7.95 (m, 1H), 7.74 − 7.63 (m, 1H), 7.45 − 7.28 (m, 5H), 5.29 − 5.19 (m, 1H), 4.72 − 4.62 (m, 1H), 4.54 − 4.38 (m, 2H), 4.13 − 3.92 (m, 1H), 3.89 − 3.49 (m, 4H), 2.43 − 2.15 (m, 3H), 2.14 − 1.79 (m, 6H), 1.75 − 1.63 (m, 1H), 1.29 − 1.13 (m, 1H), 0.98 − 0.83 (m, 1H), 0.65 − 0.50 (m, 2H), 0.46 − 0.34 (m, 1H), 0.08 − −0.04 (m, 1H) |
| | | Pyrrolidine building block SFC | | |
| | | peak 2 was used for synthesis and biological testing | | |
| 235 | ((2-(((3S,6S,9R,10aR)- 3-((3S,4R)-3- cyano-4- phenylpyrrolidine- 1-carbonyl)-9-ethyl-5- oxodecahydropyrrolo [1,2-a]azocin-6- yl)carbamoyl)benzo [b]thiophen-5- yl)fluoromethyl) phosphonic acid Or ((2(((3S,6S,9R,10aR)- 3-((3R,4S)-3-cyano-4- phenylpyrrolidine- 1-carbonyl)-9-ethyl-5- oxodecahydropyrro lo[1,2-a]azocin-6- yl)carbamoyl)benzo [b]thiophen-5- yl)fluoromethyl) phosphonic acid | 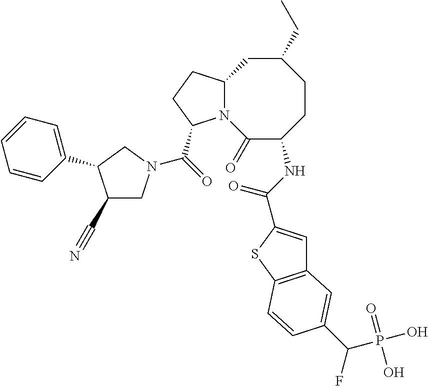 [SMILES: CC[C@@H]1CC[C@H](NC(=O)c2cc3cc(C(F)P(=O)(O)O)ccc3s2)C(=O)N2[C@H](CC[C@H]2C(=O)N2C[C@@H](C#N)[C@H](c3ccccc3)C2)C1] | 681.1 | ¹H NMR (400 MHz, CD₃OD) δ 8.14 − 7.87 (m, 3H), 7.60 − 7.58 ( m, 1H), 7.43 − 7.29 (m, 5H), 5.90 − 5.67 (m, 1H), 5.09 − 4.98 (m, 1H), 4.75 − 4.65 (m, 1H), 4.58 − 4.40 (m, 2H), 4.09 − 3.90 (m, 1H), 3.88 − 3.47 (m, 4H), 2.39 − 2.22 (m, 2H), 2.16 − 1.83 (m, 6H), 1.70 − 1.48 (m, 3H), 1.39 − 1.33 (m, 2H), 1.02 − 0.92 (m, 3H) |
| | | Or | | |
| | | 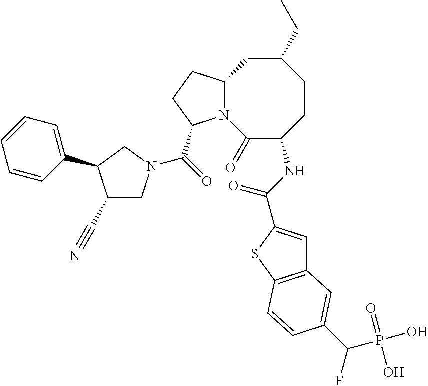 [SMILES: CC[C@@H]1CC[C@H](NC(=O)c2cc3cc(C(F)P(=O)(O)O)ccc3s2)C(=O)N2[C@H](CC[C@H]2C(=O)N2C[C@H](c3ccccc3)[C@@H](C#N)C2)C1] | | |
| | | Pyrrolidine building block SFC | | |
| | | peak 2 was used for synthesis | | |
| | | and biological testing | | |
| 236 | (fluoro(2- (((3S,6S,10aS)- 3-(3-(4- methoxypyridin-3- yl)azetidine-1- carbonyl)-5- oxodecahydropyrrolo [1,2-a]azocin-6- yl)carbamoyl)benzo [b]thiophen-5- yl)methyl) phosphonic acid | 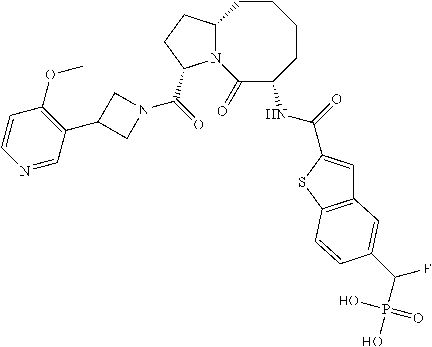 [SMILES: COc1ccncc1C1CN(C(=O)[C@@H]2CC[C@@H]3CCCC[C@H](NC(=O)c4cc5cc(C(F)P(=O)(O)O)ccc5s4)C(=O)N32)C1] | 645.1 | ¹H NMR (400 MHz, CD₃OD) δ 8.50 − 8.22 (m, 2H), 8.13 − 7.73 (m, 3H), 7.66 − 7.49 (m, 1H), 7.35 − 7.17 (m, 1H), 5.81 − 5.56 (m, 1H), 5.05 − 4.93 (m, 1H), 4.70 − 4.59 (m, 1H), 4.50 − 4.37 (m, 2H), 4.36 − 4.28 (m, 1H), 4.21 − 4.13 (m, 1H), 4.10 − 4.02 (m, 2H), 3.99 (s, 2H), 2.35 − 2.14 (m, 2H), 2.13 − 1.56 (m, 11H) |
| 237 | ((2-(((3S,6S,9S,10aR)- 3-((3R,4S)-3-cyano-4- phenylpyrrolidine- 1-carbonyl)-9-ethyl-5- oxodecahydropyrrolo [1,2-a]azocin-6- yl)carbamoyl)benzo [b]thiophen-5- yl)fluoromethyl) phosphonic acid Or ((2-(((3S,6S, 9S,10aR)- 3-((3S,4R)-3-cyano- 4-phenylpyrrolidine- 1-carbonyl)-9-ethyl-5- oxodecahydropyrrolo [1,2-a]azocin-6- yl)carbamoyl)benzo [b]thiophen-5- yl)fluoromethyl) phosphonic acid | 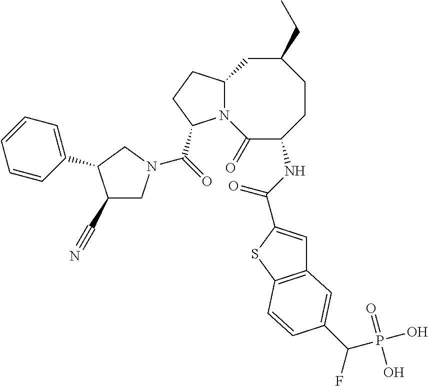 [SMILES: CC[C@H]1CC[C@H](NC(=O)c2cc3cc(C(F)P(=O)(O)O)ccc3s2)C(=O)N2[C@H](CC[C@H]2C(=O)N2C[C@@H](C#N)[C@H](c3ccccc3)C2)C1] | 681.1 | ¹H NMR (400 MHz, CD₃OD) δ 8.11 − 8.01 (m, 2H), 7.93 − 7.90 (m, 1H), 7.64 − 7.56 (m, 1H), 7.45 − 7.30 (m, 5H), 5.83 − 5.67 (m, 1H), 4.84 − 4.78 (m, 1H), 4.71 − 4.65 (m, 1H), 4.59 − 4.42 (m, 2H), 4.11 − 3.93 (m, 1H), 3.86 − 3.50 (m, 4H), 2.46 − 2.29 (m, 1H), 2.21- 2.16 (m, 1H), 2.13 − 2.02 (m, 1H), 2.00 − 1.91 (m, 1H), 1.88 − 1.62 (m, 5H), 1.46 − 1.32 (m, 4H), 0.99 − 0.93 (m, 3H) |
| | | Or | | |
| | | 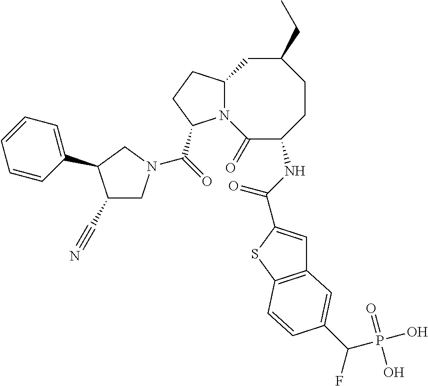 [SMILES: CC[C@H]1CC[C@H](NC(=O)c2cc3cc(C(F)P(=O)(O)O)ccc3s2)C(=O)N2[C@H](CC[C@H]2C(=O)N2C[C@H](c3ccccc3)[C@@H](C#N)C2)C1] | | |
| | | Pyrrolidine building block SFC | | |
| | | peak 2 was used for synthesis | | |
| | | and biological testing | | |
| 238 | ((2-(((3S,6S,9S,10aR)- 3-((3R,4S)-3-cyano-4- phenylpyrrolidine- 1-carbonyl)-9- isopropyl-5- oxodecahydropyrrolo [1,2-a]azocin-6- yl)carbamoyl)benzo [b]thiophen-5- yl)fluoromethyl) phosphonic acid Or ((2-(((3S,6S,9S,10aR)- 3-((3S,4R)-3-cyano-4- phenylpyrrolidine- 1-carbonyl)-9- isopropyl-5- oxodecahydropyrrolo [1,2-a]azocin-6- yl)carbamoyl)benzo [b]thiophen-5- yl)fluoromethyl) phosphonic acid | 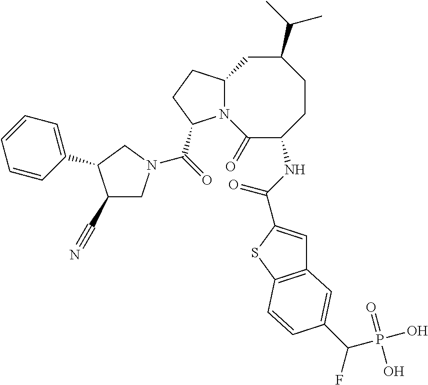 [SMILES: CC(C)[C@H]1CC[C@H](NC(=O)c2cc3cc(C(F)P(=O)(O)O)ccc3s2)C(=O)N2[C@H](CC[C@H]2C(=O)N2C[C@@H](C#N)[C@H](c3ccccc3)C2)C1] | 695.1 | ¹H NMR (400 MHz, CD₃OD) δ 8.12 − 7.87 (m, 3H), 7.64 − 7.53 (m, 1H), 7.44 − 7.27 (m, 5H), 5.89 − 5.68 (m, 1H), 4.82 − 4.75 (m, 1H), 4.71 − 4.61 (m, 1H), 4.56 − 4.38 (m, 2H), 4.11 − 3.92 (m, 1H), 3.86 − 3.51 (m, 4H), 2.45 − 2.27 (m, 1H), 2.26 − 1.99 (m, 3H), 1.93 − 1.63 (m, 6H), 1.58 − 1.42 (m, 2H), 0.97 − 0.88 (m, 6H) |
| | | Or | | |
| | | 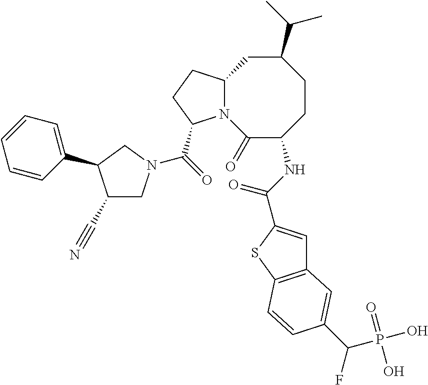 [SMILES: CC(C)[C@H]1CC[C@H](NC(=O)c2cc3cc(C(F)P(=O)(O)O)ccc3s2)C(=O)N2[C@H](CC[C@H]2C(=O)N2C[C@H](c3ccccc3)[C@@H](C#N)C2)C1] | | |
| | | Pyrrolidine building block SFC | | |
| | | peak 2 was used for synthesis | | |
| | | and biological testing | | |
| 239 | ((2-(((3S,6S,9R,10aR)- 3-((3S,4R)-3-cyano-4- phenylpyrrolidine- 1-carbonyl)-5-oxo-9- propyldeca- hydropyrrolo [1,2-a]azocin-6- yl)carbamoyl)benzo [b]thiophen-5- yl)fluoromethyl) phosphonic acid Or ((2-(((3S,6S,9R,10aR)- 3-((3R,4S)-3-cyano-4- phenylpyrrolidine- 1-carbonyl)-5-oxo-9- propyldeca- hydropyrrolo [1,2-a]azocin-6- yl)carbamoyl)benzo [b]thiophen-5- yl)fluoromethyl) phosphonic acid | 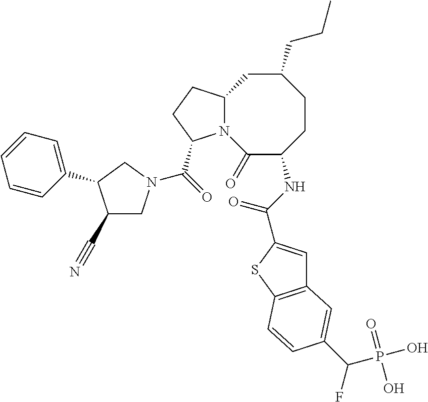 [SMILES: CCC[C@@H]1CC[C@H](NC(=O)c2cc3cc(C(F)P(=O)(O)O)ccc3s2)C(=O)N2[C@H](CC[C@H]2C(=O)N2C[C@@H](C#N)[C@H](c3ccccc3)C2)C1] | 695.1 | ¹H NMR (400 MHz, CD₃OD) δ 8.15 − 7.98 (m, 2H), 7.93 (t, J = 7.2 Hz, 1H), 7.60 − 7.58 (m, 1H), 7.44 − 7.28 (m, 5H), 5.91 − 5.65 (m, 1H), 5.04 (t, J = 9.0 Hz, 1H), 4.75 − 4.66 (m, 1H), 4.57 − 4.37 (m, 2H), 4.10 − 3.93 (m, 1H), 3.86 − 3.48 (m, 4H), 2.38 − 2.20 (m, 2H), 2.14 − 1.82 (m, 6H), 1.73 − 1.61 (m, 2H), 1.57 − 1.50 (m, 1H), 1.45 − 1.30 (m, 4H), 0.96 − 0.92 (m, 3H) |
| | | Or | | |
| | | 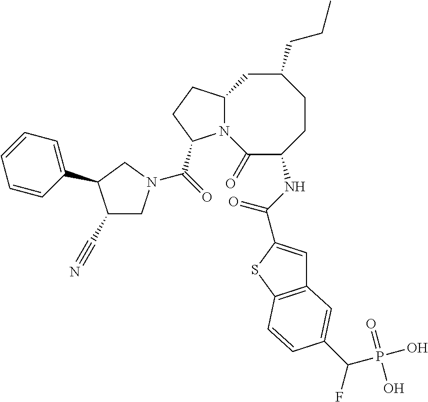 [SMILES: CCC[C@@H]1CC[C@H](NC(=O)c2cc3cc(C(F)P(=O)(O)O)ccc3s2)C(=O)N2[C@H](CC[C@H]2C(=O)N2C[C@H](c3ccccc3)[C@@H](C#N)C2)C1] | | |
| | | Pyrrolidine building block SFC | | |
| | | peak 2 was used for synthesis | | |
| | | and biological testing | | |
| 240 | ((2-(((3S,6S,9S,10aR)- 3-((3S,4R)-3-cyano-4- phenylpyrrolidine- 1-carbonyl)-5-oxo-9- propyldeca- hydropyrrolo [1,2-a]azocin-6- yl)carbamoyl)benzo [b]thiophen-5- yl)fluoromethyl) phosphonic acid Or ((2-(((3S,6S,9S,10aR)- 3-((3R,4S)-3-cyano-4- phenylpyrrolidine- 1-carbonyl)-5-oxo-9- propyldeca- hydropyrrolo [1,2-a]azocin-6- yl)carbamoyl)benzo [b]thiophen-5- yl)fluoromethyl) phosphonic acid | 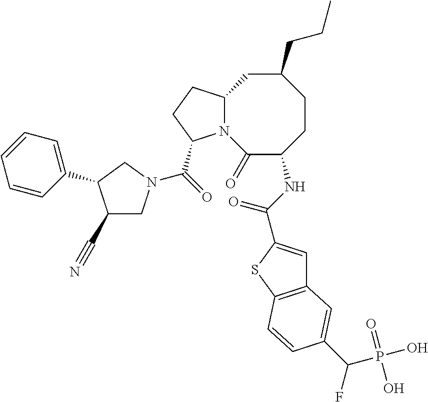 [SMILES: CCC[C@H]1CC[C@H](NC(=O)c2cc3cc(C(F)P(=O)(O)O)ccc3s2)C(=O)N2[C@H](CC[C@H]2C(=O)N2C[C@@H](C#N)[C@H](c3ccccc3)C2)C1] | 695.1 | ¹H NMR (400 MHz, CD₃OD) δ 8.12 − 8.01 (m, 2H), 7.95 − 7.92 (m 1H), 7.60 − 7.58 (m, 1H), 7.45 − 7.28 (m, 5H), 5.90 − 5.71 (m, 1H), 4.84 − 4.81 (m, 1H), 4.71 − 4.63 (m, 1H), 4.58 − 4.42 (m, 2H), 4.12 − 3.93 (m, 1H), 3.87 − 3.47 (m, 4H), 2.44 − 2.24 (m, 1H), 2.24 − 1.91 (m, 4H), 1.87 − 1.77 (m, 3H), 1.72 − 1.61 (m, 1H), 1.47 − 1.26 (m, 6H), 0.97 − 0.89 (m, 3H) |
| | | Or | | |
| | | 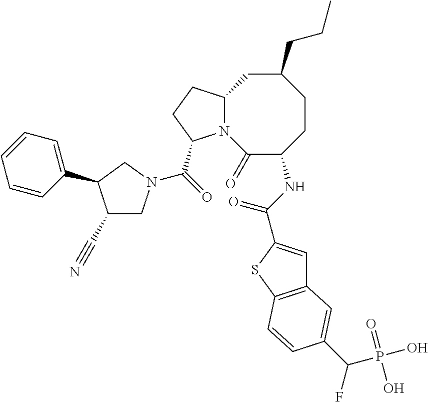 [SMILES: CCC[C@H]1CC[C@H](NC(=O)c2cc3cc(C(F)P(=O)(O)O)ccc3s2)C(=O)N2[C@H](CC[C@H]2C(=O)N2C[C@H](c3ccccc3)[C@@H](C#N)C2)C1] | | |
| | | Pyrrolidine building block SFC | | |
| | | peak 2 was used for synthesis | | |
| | | and biological testing | | |
| 241 | (fluoro(2- (((3S,6S,9R,10aR)- 3-(3-(4- methoxypyridin-3- yl)azetidine-1- carbonyl)-5-oxo-9- propyldeca- hydropyrrolo [1,2-a]azocin-6- yl)carbamoyl)benzo [b]thiophen-5- yl)methyl)phosphonic acid | 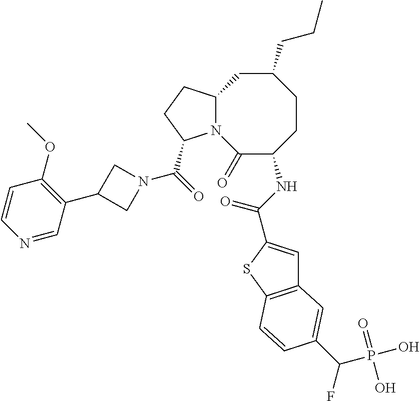 [SMILES: CCC[C@@H]1CC[C@H](NC(=O)c2cc3cc(C(F)P(=O)(O)O)ccc3s2)C(=O)N2[C@H](CC[C@H]2C(=O)N2CC(c3cnccc3OC)C2)C1] | 687.1 | ¹H NMR (400 MHz, CD₃OD) δ 8.40 − 8.26 (m, 2H), 8.13 − 7.96 (m, 2H), 7.88 − 7.83 (m, 1H), 7.67 − 7.58 (m, 1H), 7.11 − 6.97 (m, 1H), 5.74 − 5.55 (m, 1H), 5.09 − 5.01 (m, 1H), 4.63 − 4.59 (m, 1H), 4.52 − 4.37 (m, 4H), 4.34 − 4.19 (m, 1H), 4.14 − 4.05 (m, 1H), 3.94 − 3.91 (m, 3H), 2.34 − 2.19 (m, 2H), 2.13 − 1.76 (m, 6H), 1.71 − 1.59 (m, 2H), 1.56 − 1.28 (m, 5H), 0.94 (t, J = 7.0 Hz, 3H) |
| 242 | ((R)-(2- (((3S,6S,10aS)-3- ((3R,4S)-3-cyano-4- phenylpyrrolidine- 1-carbonyl)-5- oxodecahydropyrrolo [1,2-a]azocin-6- yl)carbamoyl)benzo [b]thiophen-5- yl)fluoromethyl) phosphonic acid Or ((R)-(2-(((3S, 6S,10aS)- 3-((3S,4R)-3-cyano- 4-phenylpyrrolidine- 1-carbonyl)-5- oxodecahydropyrrolo [1,2-a]azocin-6- yl)carbamoyl)benzo [b]thiophen-5- yl)fluoromethyl) phosphonic acid | 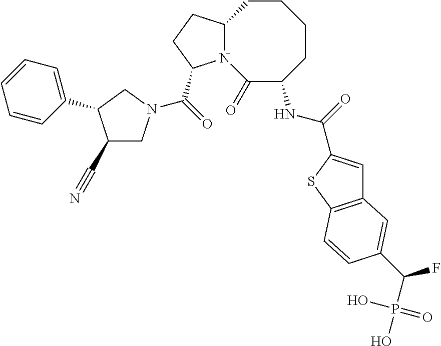 [SMILES: N#C[C@@H]1CN(C(=O)[C@@H]2CC[C@@H]3CCCC[C@H](NC(=O)c4cc5cc([C@@H](F)P(=O)(O)O)ccc5s4)C(=O)N32)C[C@H]1c1ccccc1] Or | 653.2 | ¹H NMR (400 MHz, CD₃OD) δ 8.14 − 8.09 (m, 1H), 8.06 − 8.00 (m, 1H), 7.99 − 7.91 (m, 1H), 7.63 − 7.55 (m, 1H), 7.46 − 7.29 (m, 5H), 5.93 − 5.73 (m, 1H), 5.09 − 4.99 (m, 1H), 4.75 − 4.63 (m, 1H), 4.58 − 4.37 (m, 2H), 4.12 − 3.91 (m, 1H), 3.89 − 3.65 (m, 2H), 3.63 − 3.49 (m, 2H), 2.43 − 2.15 (m, 2H), 2.12 − 1.90 (m, 6H), 1.90 − 1.78 (m, 2H), 1.73 − 1.59 (m, 2H) |
| | | 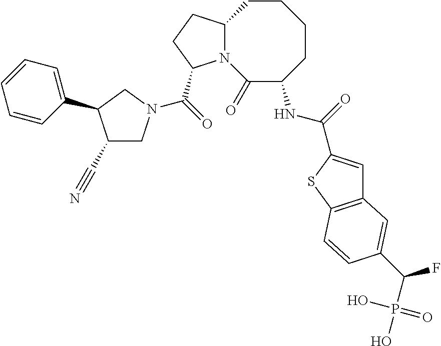 [SMILES: N#C[C@H]1CN(C(=O)[C@@H]2CC[C@@H]3CCCC[C@H](NC(=O)c4cc5cc([C@@H](F)P(=O)(O)O)ccc5s4)C(=O)N32)C[C@@H]1c1ccccc1] | | |
| | | Pyrrolidine building block SFC | | |
| | | peak 2 was used for synthesis | | |
| | | and biological testing | | |
| 243 | ((R)-(2-(((3S,6S,10aS)- 3-((3R,4S)-3-cyano-4- phenylpyrrolidine- 1-carbonyl)-5- oxodecahydropyrro lo[1,2-a]azocin-6- yl)carbamoyl)benzo [b]thiophen-5- yl)fluoromethyl) phosphonic acid Or ((R)-(2- (((3S,6S,10aS)-3- ((3S,4R)-3-cyano-4- phenylpyrrolidine- 1-carbonyl)-5- oxodecahydropyrrolo [1,2-a]azocin-6- yl)carbamoyl)benzo [b]thiophen-5- yl)fluoromethyl) phosphonic acid | 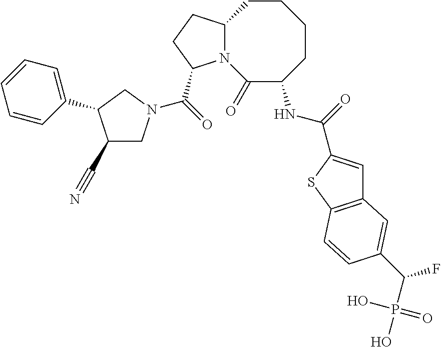 [SMILES: N#C[C@@H]1CN(C(=O)[C@@H]2CC[C@@H]3CCCC[C@H](NC(=O)c4cc5cc([C@H](F)P(=O)(O)O)ccc5s4)C(=O)N32)C[C@H]1c1ccccc1] Or | 653.2 | ¹H NMR (400 MHz, CD₃OD) δ 8.14 − 8.09 (m, 1H), 8.06 − 7.99 (m, 1H), 7.98 − 7.91 (m, 1H), 7.63 − 7.53 (m, 1H), 7.43 − 7.30 (m, 5H), 5.93 − 5.73 (m, 1H), 5.09 − 4.96 (m, 1H), 4.75 − 4.62 (m, 1H), 4.59 − 4.38 (m, 2H), 4.11 − 3.93 (m, 1H), 3.88 − 3.67 (m, 2H), 3.64 − 3.50 (m, 2H), 2.42 − 2.17 (m, 2H), 2.11 − 1.95 (m, 6H), 1.91 − 1.76 (m, 2H), 1.75 − 1.59 (m, 2H) |
| | | 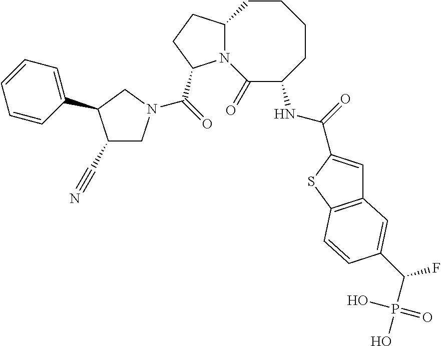 [SMILES: N#C[C@H]1CN(C(=O)[C@@H]2CC[C@@H]3CCCC[C@H](NC(=O)c4cc5cc([C@H](F)P(=O)(O)O)ccc5s4)C(=O)N32)C[C@@H]1c1ccccc1] | | |
| | | Pyrrolidine building block SFC | | |
| | | peak 2 was used for synthesis | | |
| | | and biological testing | | |
| 244 | ((2-(((3S,6S,10aS)- 5-ox0-3-((R)-6- (pyridin-3-yl)-4- azaspiro[2.4]heptane- 4-carbonyl) decahydropyrrolo[1,2- a]azocin-6- yl)carbamoyl)benzo [b]thiophen-5- yl)methyl) phosphonic acid | 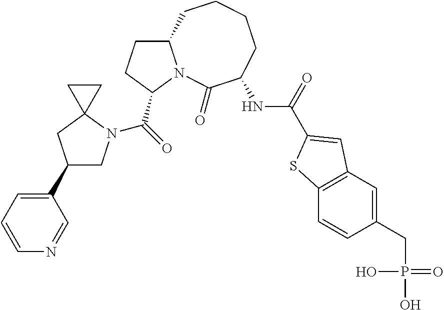 [SMILES: O=C(N[C@H]1CCCC[C@H]2CC[C@@H](C(=O)N3C[C@@H](c4cccnc4)CC34CC4)N2C1=O)c1cc2cc(CP(=O)(O)O)ccc2s1] | 637.3 | ¹H NMR (400 MHz, DMSO) δ 8.78 (d, J = 7.3 Hz, 1H), 8.60 − 8.53 (m, 1H), 8.46 − 8.39 (m, 1H), 8.19 (s, 1H), 7.89 (d, J = 8.4 Hz, 1H), 7.82 − 7.74 (m, 2H), 7.37 − 7.29 (m, 2H), 4.99 − 4.87 (m, 1H), 4.55 − 4.44 (m, 1H), 4.26 − 4.19 (m, 1H), 4.18 − 4.09 (m, 1H), 3.84 − 3.77 (m, 1H), 3.61 − 3.55 |
| | | | | (m, 1H), 3.09 (d, |
| | | | | J = 21.2 Hz, |
| | | | | 2H), 2.34 − 2.21 |
| | | | | (m, 1H), 2.19 − |
| | | | | 2.07 (m, 2H), |
| | | | | 2.06 − 1.93 (m, |
| | | | | 3H), 1.91 − 1.68 |
| | | | | (m, 6H), 1.66 − |
| | | | | 1.44 (m, 4H), |
| | | | | 0.52 − 0.38 (m, 2H) |
| 245 | ((2-(((3S,6S,9S,10aR)- 9-ethyl-3-(3-(4- methoxypyridin-3- yl)azetidine-1- carbonyl)-5- oxodecahydropyrrolo [1,2-a]azocin-6- yl)carbamoyl)benzo [b]thiophen-5- yl)fluoromethyl) phosphonic acid | 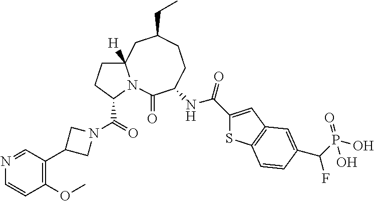 [SMILES: CC[C@H]1CC[C@H](NC(=O)c2cc3cc(C(F)P(=O)(O)O)ccc3s2)C(=O)N2[C@H](CC[C@H]2C(=O)N2CC(c3cnccc3OC)C2)C1] | 673.1 | ¹H NMR (400 MHz, CD₃OD) δ 8.40 − 8.23 (m, 2H), 8.09 − 7.91 (m, 2H), 7.87 − 7.76 (m, 1H), 7.66 − 7.54 (m, 1H), 7.14 − 6.99 (m, 1H), 5.72 − 5.56 (m, 1H), 4.71 − 4.58 (m, 2H), 4.50 − 4.36 (m, 3H), 4.35 − |
| | | | | 4.19 (m, 1H), |
| | | | | 4.14 − 4.00 (m, |
| | | | | 2H), 3.95 − 3.90 |
| | | | | (m, 3H), 2.36 − |
| | | | | 2.26 (m, 1H), |
| | | | | 2.24 − 1.92 (m, |
| | | | | 4H), 1.88 − 1.60 |
| | | | | (m, 5H), 1.44 − |
| | | | | 1.32 (m, 3H), |
| | | | | 1.00 − 0.89 (m, 3H) |
| 246 | (fluoro(2- (((3S,6S,9S,10aR)- 9-isopropyl-3-(3- (4-methoxypyridin- 3-yl)azetidine-1- carbonyl)-5- oxodecahydropyrrolo [1,2-a]azocin-6- yl)carbamoyl)benzo [b]thiophen-5- yl)methyl) phosphonic acid | 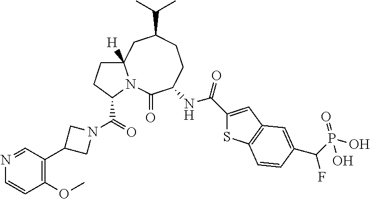 [SMILES: COc1ccncc1C1CN(C(=O)[C@@H]2CC[C@@H]3C[C@@H](C(C)C)CC[C@H](NC(=O)c4cc5cc(C(F)P(=O)(O)O)ccc5s4)C(=O)N32)C1] | 687.1 | ¹H NMR (400 MHz, CD₃OD) δ 8.38 − 8.29 (m, 2H), 8.11 − 8.02 (m, 2H), 7.83 − 7.69 (m, 2H), 7.10 − 7.04 (m, 1H), 5.68 − 5.54 (m, 1H), 4.77 − 4.57 (m, 3H), 4.49 − 4.36 (m, 3H), 4.34 − 4.21 (m, 1H), 4.15 − |
| | | | | 4.06 (m, 1H), |
| | | | | 3.95 (s, 3H), |
| | | | | 2.38 − 2.28 (m, |
| | | | | 1H), 2.25 − 2.12 |
| | | | | (m, 2H), 2.10 − |
| | | | | 1.99 (m, 1H), |
| | | | | 1.89 − 1.73 (m, |
| | | | | 4H), 1.73 − 1.62 |
| | | | | (m, 2H), 1.58 − |
| | | | | 1.42 (m, 2H), |
| | | | | 0.93 − 0.88 (m, 6H) |
| 247 | (fluoro(2- (((3S,6S,9S,10aR)- 3-(3-(4- methoxypyridin-3- yl)azetidine-1- carbonyl)-5-oxo-9- propyldeca- hydropyrrolo [1,2-a]azocin-6- yl)carbamoyl)benzo [b]thiophen-5- yl)methyl)phosphonic acid | 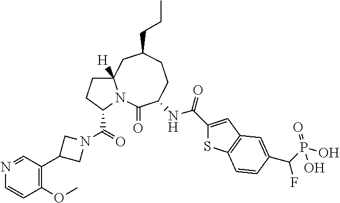 [SMILES: CCC[C@H]1CC[C@H](NC(=O)c2cc3cc(C(F)P(=O)(O)O)ccc3s2)C(=O)N2[C@H](CC[C@H]2C(=O)N2CC(c3cnccc3OC)C2)C1] | 687.1 | ¹H NMR (400 MHz, CD₃OD) δ 8.39 − 8.25 (m, 2H), 8.12 − 8.01 (m, 2H), 7.83 − 7.67 (m, 2H), 7.10 − 7.05 (m, 1H), 5.69 − 5.51 (m, 1H), 4.95 − 4.88 (m, 2H), 4.73 − 4.57 (m, 1H), 4.49 − 4.20 (m, 4H), 4.17 − |
| | | | | 4.06 (m, 1H), |
| | | | | 3.97 − 3.93 (m, |
| | | | | 3H), 2.38 − 2.26 |
| | | | | (m, 1H), 2.24 − |
| | | | | 2.08 (m, 2H), |
| | | | | 2.07 − 1.88 (m, |
| | | | | 2H), 1.86 − 1.72 |
| | | | | (m, 4H), 1.75 − |
| | | | | 1.61 (m, 1H), |
| | | | | 1.46 − 1.26 (m, |
| | | | | 5H), 0.95 − 0.91 |
| | | | | (m, 3H) |

### Compounds Prepared According to General Scheme 1: Phosphonate Esters

The following compounds in Table 108 were prepared according to the representative procedure described above for the synthesis of (difluoro(2-(((3S,6S,10aS)-3-(methyl(phenyl)carbamoyl)-5-oxodecahydropyrrolo[1,2-a]azocin-6-yl)carbamoyl)-1H-indol-5-yl)methyl)phosphonic acid and utilizing the appropriate starting materials and modifications.

TABLE 108

| | | | LCMS | |
|---|---|---|---|---|
| Compound | Name | Structure | [M + H]⁺ | NMR |
| 248 | isopropyl ((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 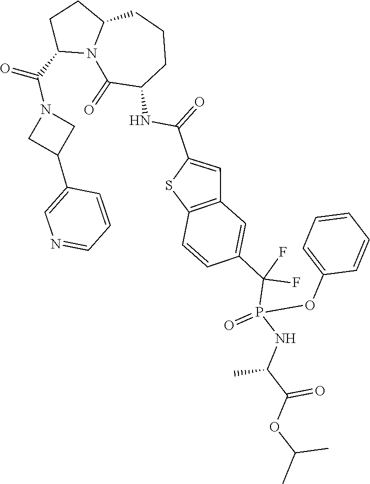 [SMILES: CC(C)OC(=O)[C@H](C)NP(=O)(Oc1ccccc1)C(F)(F)c1ccc2sc(C(=O)N[C@H]3CCC[C@H]4CC[C@@H](C(=O)N5CC(c6cccnc6)C5)N4C3=O)cc2c1] | 808.2 | ¹H NMR (400 MHz, CDCl₃) δ 9.10-8.83 (m, 1H), 8.78-8.69 (m, 1H), 8.45 (m, 1H), 8.19-7.99 (m, 2H), 7.97-7.83 (m, 2H), 7.82-7.63 (m, 2H), 7.31-7.28 (m, 1H), 7.18-7.08 (m, 3H), 5.07-4.94 (m, 1H), 4.89-4.78 (m, 1H), 4.76-4.69 (m, 1H), 4.57-4.45 (m, 2H), 4.29-4.08 (m, 2H), 4.06-3.86 (m, 3H), 2.35-2.25 (m, 1H), 2.20-2.13 (m, 1H), 2.12-1.99 (m, 4H), 1.98-1.76 (m, 4H), 1.39-1.30 (m, 3H), 1.27-1.16 (m, 6H) |
| 249 | (((difluoro(2- (((3S,6S)-3-(3- morpholinoazetidine- 1-carbonyl)-5- oxooctahydro-1H- pyrrolo[1,2- a]azepin-6- yl)carbamoyl)benzo [b]thiophen-5- yl)methyl)phosphoryl) bis(oxy))bis (methylene) bis(2,2- dimethylpropanoate) | 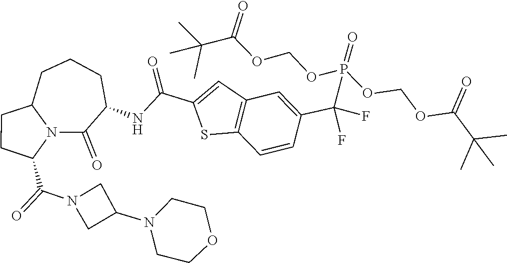 [SMILES: CC(C)(C)C(=O)OCOP(=O)(OCOC(=O)C(C)(C)C)C(F)(F)c1ccc2sc(C(=O)N[C@H]3CCCC4CC[C@@H](C(=O)N5CC(N6CCOCC6)C5)N4C3=O)cc2c1] | 855.3 | ¹H NMR (400 MHz, CD₃OD) δ 8.17 (d, J = 5.6 Hz, 2H), 8.13- 8.05 (m, 1H), 7.64 (d, J = 8.8 Hz, 1H), 5.82-5.69 (m, 4H), 4.77 (d, J = 10.8 Hz, 1H), 4.63-4.48 (m, 2H), 4.43-4.27 (m, 1H), 4.17-4.05 (m, 2H), 3.96-3.82 (m, 1H), 3.72 (td, J = 4.4, 13.6 Hz, 4H), 3.30- 3.22 (m, 1H), 2.46 (s, 4H), 2.37-2.27 (m, 1H), 2.11 (d, J = 18.0 Hz, 3H), 1.99-1.82 (m, 6H), 1.22-1.17 (m, 18H) |
| 250 | propyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 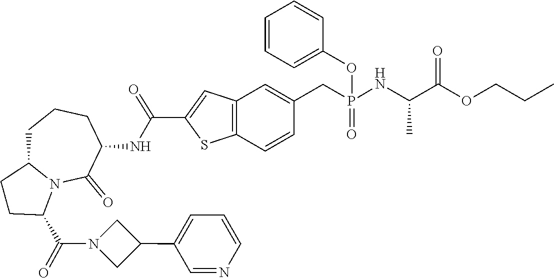 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 772.6 | ¹H NMR (400 MHz, CDCl₃) δ 8.70-8.54 (m, 2H), 8.08-7.85 (m, 1H), 7.84-7.75 (m, 3H), 7.74-7.63 (m, 1H), 7.53-7.44 (m, 1H), 7.44-7.37 (m, 1H), 7.33-7.27 (m, 2H), 7.19-7.10 (m, 3H), 4.73-4.63 (m, 2H), 4.61-4.52 (m, 1H), 4.27-4.15 (m, 1H), 4.07-3.88 (m, 5H), 3.52-3.31 (m, 3H), 3.27-3.08 (m, 1H), 2.98-2.84 (m, 2H), 2.34-2.14 (m, 2H), 2.10-2.00 (m, 3H), 1.97-1.92 (m, 1H), 1.86-1.68 (m, 2H), 1.63-1.50 (m, 2H), 1.47-1.40 (m, 1H), 1.16 (dd, J = 7.2, 10.4 Hz, 3H), 0.92-0.82 (m, 3H) |
| 251 | propyl (((2- (((3S,6S,9aR)-8- methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 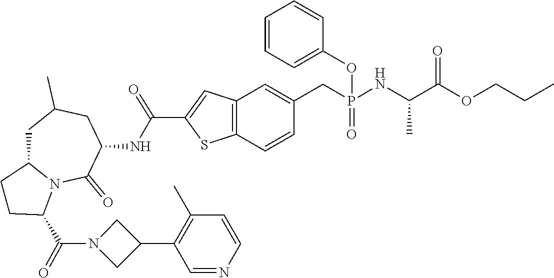 | 800.4 | ¹H NMR (400 MHz, CD₃OD) δ 8.88- 8.81 (m, 1H), 8.66- 8.54 (m, 1H), 8.09- 7.99 (m, 1H), 7.95- 7.83 (m, 3H), 7.54- 7.44 (m, 1H), 7.37- 7.27 (m, 2H), 7.16 (t, J = 8.4 Hz, 3H), 5.04- 4.92 (m, 1H), 4.77- 4.72 (m, 1H), 4.66 (t, J = 7.2 Hz, 1H), 4.59- 4.52 (m, 1H), 4.49- 4.41 (m, 1H), 4.41- 4.33 (m, 1H), 4.22- 4.05 (m, 2H), 3.98- 3.81 (m, 3H), 3.58- 3.45 (m, 2H), 2.57 (d, J = 4.0 Hz, 3H), 2.32 (m, 1H), 2.14- 1.89 (m, 5H), 1.86- 1.76 (m, 1H), 1.69- 1.49 (m, 4H), 1.26- 1.21 (m, 1H), 1.14 (d, J = 6.8 Hz, 1H), 1.07 (m, 4H), 0.87 (t, J = 7.2 Hz, 3H) |
| 252 | propyl (((2- (((3S,6S,9aS)-5- oxo-3-(3-(pyridin- 3-yl)pyrrolidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 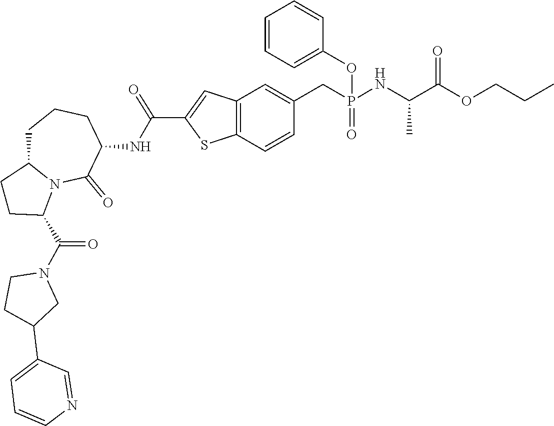 [SMILES: CCCOC(=O)[C@H](C)NP(=O)(Cc1ccc2sc(C(=O)N[C@H]3CCC[C@H]4CC[C@@H](C(=O)N5CCC(c6cccnc6)C5)N4C3=O)cc2c1)Oc1ccccc1] | 786.4 | ¹H NMR (400 MHz, CDCl₃) δ 8.81-8.50 (m, 2H), 8.19-7.96 (m, 1H), 7.92-7.76 (m, 3H), 7.67 (d, J = 4.0 Hz, 1H), 7.54- 7.30 (m, 4H), 7.21- 7.11 (m, 3H), 4.83- 4.68 (m, 2H), 4.39- 4.13 (m, 1H), 4.01- 3.86 (m, 4H), 3.75- 3.60 (m, 1H), 3.54- 3.40 (m, 3H), 3.33- 3.10 (m, 1H), 2.56- 2.40 (m, 1H), 2.33- 2.25 (m, 1H), 2.22- 2.18 (m, 1H), 2.12- 2.03 (m, 4H), 1.96- 1.83 (m, 3H), 1.79- 1.68 (m, 2H), 1.66- 1.48 (m, 1H), 1.23- 1.12 (m, 3H), 0.89 (d, J = 7.4 Hz, 3H) |
| 253 | propyl (((2- (((3S,6S,10aS)-5- oxo-3-(3-(pyridin- 3-yl)pyrrolidine-1- carbonyl)decahydro pyrrolo[1,2- a]azocin-6- yl)carbamoyl)benzo [b]thiophen-5- yl)methyl)(phenoxy) phosphoryl)-L- alaninate | 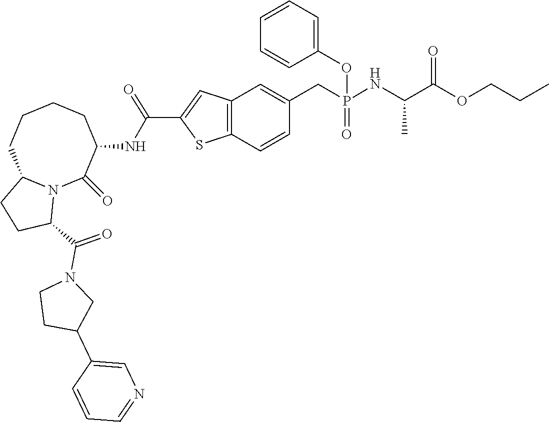 [SMILES: CCCOC(=O)[C@H](C)NP(=O)(Cc1ccc2sc(C(=O)N[C@H]3CCCC[C@H]4CC[C@@H](C(=O)N5CCC(c6cccnc6)C5)N4C3=O)cc2c1)Oc1ccccc1] | 800.5 | ¹H NMR (400 MHz, CDCl₃) δ 8.54-8.36 (m, 2H), 7.79-7.66 (m, 3H), 7.60-7.30 (m, 3H), 7.25-7.20 (m, 3H), 7.16-7.02 (m, 3H), 5.13-5.02 (m, 1H), 4.65-4.49 (m, 1H), 4.34-4.06 (m, 2H), 3.96-3.73 (m, 4H), 3.51-3.31 (m, 4H), 3.25-3.01 (m, 1H), 2.42-2.18 (m, 2H), 2.14-1.94 (m, 7H), 1.79-1.58 (m, 3H), 1.47 (s, 2H), 1.12-1.03 (m, 3H), 0.88-0.76 (m, 3H) |
| 254 | butyl ((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 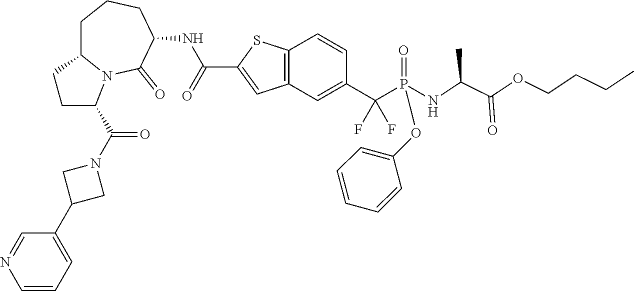 [SMILES: CCCCOC(=O)[C@H](C)NP(=O)(Oc1ccccc1)C(F)(F)c1ccc2sc(C(=O)N[C@H]3CCC[C@H]4CC[C@@H](C(=O)N5CC(c6cccnc6)C5)N4C3=O)cc2c1] | 822.0 | ¹H NMR (400 MHz, CDCl₃) δ 8.84-9.12 (m, 1H), 8.69-8.82 (m, 1H), 8.44-8.54 (m, 1H), 8.15 (s, 1H), 7.74-7.99 (m, 4H), 7.65-7.74 (m, 1H), 7.28 (s, 1H), 7.05- 7.19 (m, 3H), 4.64- 4.91 (m, 3H), 4.50 (m, 2H), 4.11 (m, 3H), 3.96 (m, 3H), 2.24-2.35 (m, 1H), 2.02-2.21 (m, 5H), 1.82-1.97 (m, 3H), 1.59 (s, 2H), 1.26- 1.41 (m, 6H), 0.91 (m, 3H) |
| 255 | propyl (((2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 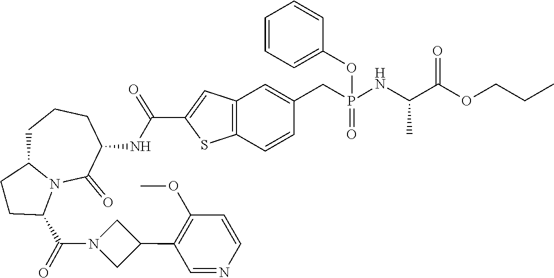 [SMILES: CCCOC(=O)[C@H](C)NP(=O)(Cc1ccc2sc(C(=O)N[C@H]3CCC[C@H]4CC[C@@H](C(=O)N5CC(c6cnccc6OC)C5)N4C3=O)cc2c1)Oc1ccccc1] | 802.2 | ¹H NMR (400 MHz, CD₃OD) δ 8.71- 8.52 (m, 2H), 8.05- 7.94 (m, 1H), 7.92- 7.79 (m, 2H), 7.65- 7.43 (m, 2H), 7.36- 7.26 (m, 2H), 7.21- 7.09 (m, 3H), 4.72 (d, J = 9.2 Hz, 1H), 4.64 (d, J = 7.6 Hz, 1H), 4.58-4.51 (m, 1H), 4.43 (s, 1H), 4.35 (s, 1H), 4.20- 4.12 (m, 5H), 4.07 (d, J = 5.2 Hz, 1H), 4.00-3.78 (m, 3H), 3.64-3.39 (m, 2H), 2.40-2.23 (m, 1H), 2.20-1.73 (m, 10H), 1.61-1.45 (m, 2H), 1.17-1.07 (m, 3H), 0.92-0.79 (m, 3H) |
| 256 | neopentyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 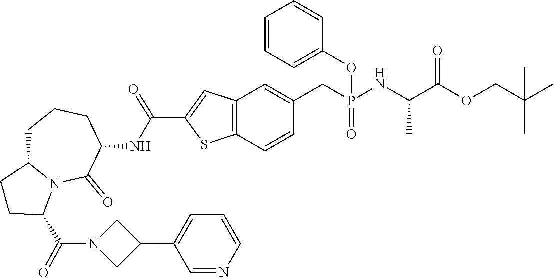 [SMILES: C[C@H](NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1)C(=O)OCC(C)(C)C] | 800.2 | ¹H NMR (400 MHz, CDCl₃) δ 9.02-8.82 (m, 1H), 8.79-8.69 (m, 1H), 8.50 (d, J = 7.6 Hz, 1H), 7.89- 7.73 (m, 5H), 7.47- 7.38 (m, 1H), 7.33- 7.29 (m, 1H), 7.15 (d, J = 8.0 Hz, 3H), 4.84-4.68 (m, 3H), 4.56-4.46 (m, 2H), 4.27-4.15 (m, 1H), 4.10-4.02 (m, 1H), 3.99 (d, J = 7.2 Hz, 2H), 3.81-3.75 (m, 1H), 3.67-3.62 (m, 1H), 3.52-3.48 (m, 1H), 3.44 (d, J = 13.2 Hz, 1H), 2.34-2.26 (m, 1H), 2.20-2.03 (m, 5H), 1.97-1.70 (m, 4H), 1.22-1.15 (m, 3H), 0.89-0.86 (m, 9H) |
| 257 | propyl (((2- (((3S,6S,9aS)-3-(3- (4-ethylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 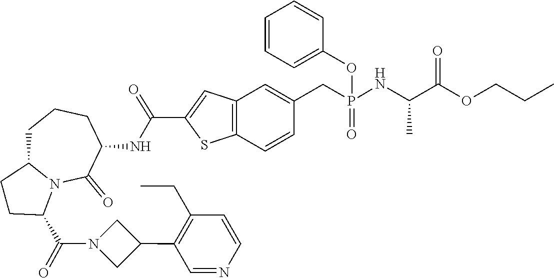 [SMILES: CCCOC(=O)[C@H](C)NP(=O)(Cc1ccc2sc(C(=O)N[C@H]3CCC[C@H]4CC[C@@H](C(=O)N5CC(c6cnccc6CC)C5)N4C3=O)cc2c1)Oc1ccccc1] | 800.3 | ¹H NMR (400 MHz, CDCl₃) δ 9.13-8.82 (m, 1H), 8.63-8.52 (m, 1H), 8.19-7.89 (m, 1H), 7.60-7.51 (m, 1H), 7.87-7.50 (m, 3H), 7.48-7.35 (m, 1H), 7.32-7.28 (m, 1H), 7.20-7.08 (m, 3H), 5.08-4.90 (m, 1H), 4.86-4.61 (m, 2H), 4.54 (t, J = 5.1 Hz, 1H), 4.45- 4.35 (m, 1H), 4.28- 4.17 (m, 1H), 4.09- 3.89 (m, 7H), 3.74- 3.63 (m, 1H), 3.51- 3.40 (m, 2H), 2.80- 2.67 (m, 2H), 2.33- 2.12 (m, 2H), 2.11- 1.97 (m, 4H), 1.93- 1.79 (m, 3H), 1.64- 1.51 (m, 2H), 1.31- 1.21 (m, 3H), 1.31- 1.12 (m, 3H), 0.93- 0.83 (m, 3H) |
| 258 | 2-ethylbutyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 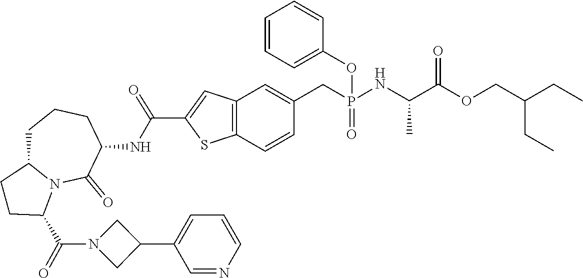 [SMILES: CCC(CC)COC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 814.3 | ¹H NMR (400 MHz, CDCl₃) δ 8.60 (d, J = 15.2 Hz, 1H), 7.98- 7.76 (m, 4H), 7.73- 7.64 (m, 1H), 7.50- 7.42 (m, 1H), 7.41- 7.34 (m, 1H), 7.32- 7.27 (m, 2H), 7.20- 7.10 (m, 3H), 4.72- 4.40 (m, 4H), 4.21 (m, 1H), 4.10-3.98 (m, 2H), 3.97-3.93 (m, 2H), 3.92-3.86 (m, 1H), 3.51-3.40 (m, 2H), 3.36-3.10 (m, 1H), 2.33-2.26 (m, 1H), 2.25-2.19 (m, 1H), 2.10-2.03 (m, 4H), 1.96 (t, J = 10.0 Hz, 2H), 1.85- 1.70 (m, 2H), 1.48- 1.41 (m, 1H), 1.34- 1.28 (m, 4H), 1.26 (s, 3H), 0.88-0.82 (m, 6H) |
| 259 | isobutyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 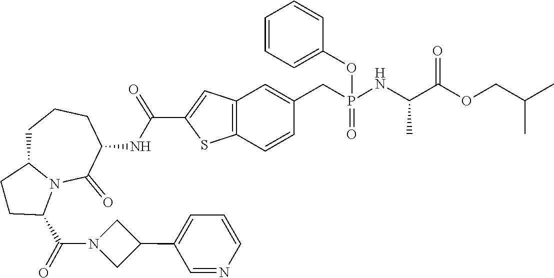 [SMILES: CC(C)COC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 786.4 | ¹H NMR (400 MHz, CDCl₃) δ 8.66-8.56 (m, 1H), 8.09-7.96 (m, 1H), 7.91-7.76 (m, 3H), 7.72-7.59 (m, 1H), 7.56-7.45 (m, 1H), 7.44-7.37 (m, 1H), 7.33-7.28 (m, 2H), 7.21-7.10 (m, 3H), 4.79-4.71 (m, 1H), 4.70-4.61 (m, 1H), 4.60-4.51 (m, 1H), 4.50-4.40 (m, 1H), 4.25-4.16 (m, 1H), 4.04-3.88 (m, 3H), 3.85-3.71 (m, 2H), 3.52-3.39 (m, 2H), 3.37-3.25 (m, 1H), 2.31-2.19 (m, 2H), 2.10-2.01 (m, 4H), 1.98-1.91 (m, 3H), 1.88-1.82 (m, 2H), 1.21-1.13 (m, 3H), 0.90-0.83 (m, 6H) |
| 260 | propyl (((2- (((3S,6S,9aS)-3-(3- (4- methylpyrimidin-5- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 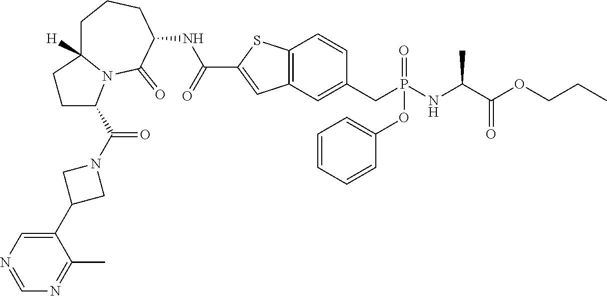 [SMILES: CCCOC(=O)[C@H](C)NP(=O)(Cc1ccc2sc(C(=O)N[C@H]3CCC[C@H]4CC[C@@H](C(=O)N5CC(c6cncnc6C)C5)N4C3=O)cc2c1)Oc1ccccc1] | 787.1 | ¹H NMR (400 MHz, CDCl₃) δ 9.08 ( d, J = 12.0 Hz, 1H), 8.92- 8.71 (m, 1H), 7.88- 7.77 (m, 3H), 7.67- 7.61 (m, 1H), 7.49- 7.38 (m, 1H), 7.32- 7.28 (m, 1H), 7.18- 7.09 (m, 3H), 5.10- 4.77 (m, 1H), 4.75- 4.61 (m, 2H), 4.59- 4.40 (m, 1H), 4.34- 4.25 (m, 1H), 4.17- 4.08 (m, 1H), 4.03- 3.88 (m, 4H), 3.55- 3.18 (m, 3H), 2.53 (d, J = 5.6 Hz, 3H), 2.34-2.13 (m, 2H), 2.12-1.99 (m, 4H), 1.931.91 (m, 2H), 1.83 ( s, 1H), 1.76- 1.66 (m, 1H), 1.62- 1.50 (m, 2H), 1.20- 1.12 (m, 3H), 0.92- 0.82 (m, 3H) |
| 261 | propyl (((2- (((3S,6S,9aS)-3-(3- (4-ethylpyridin-3- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 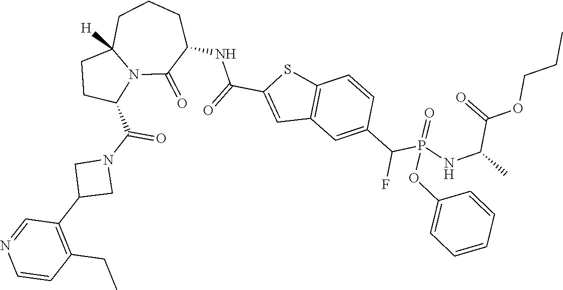 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cnccc6CC)C5)N4C3=O)cc2c1] | 818.3 | ¹H NMR (400 MHz, CD₃OD) δ 8.76- 8.61 (m, 1H), 8.42- 8.33 (m, 1H), 8.15- 7.94 (m, 3H), 7.70- 7.58 (m, 1H), 7.38- 7.29 (m, 3H), 7.24- 7.14 (m, 3H), 6.21- 5.99 (m, 1H), 4.99- 4.93 (m, 1H), 4.78- 4.70 (m, 2H), 4.63- 4.49 (m, 2H), 4.45- 4.37 (m, 1H), 4.32- 4.24 (m, 1H), 4.16- 4.06 (m, 2H), 4.00- 3.93 (m, 1H), 3.88- 3.79 (m, 1H), 2.71- 2.65 (m, 2H), 2.32- 1.98 (m, 6H), 1.93- 1.80 (m, 4H), 1.61- 1.48 (m, 2H), 1.30- 1.20 (m, 6H), 0.93- 0.83 (m, 3H) |
| 262 | propyl ((fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 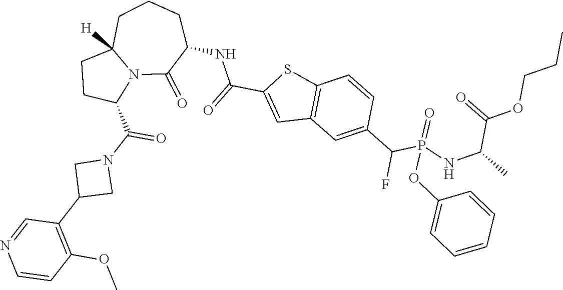 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cnccc6OC)C5)N4C3=O)cc2c1] | 820.7 | ¹H NMR (400 MHz, ACETONITRILE- d₃) δ 8.62-8.49 (m, 2H), 8.07-7.91 (m, 3H), 7.90-7.73 (m, 1H), 7.65-7.56 (m, 1H), 7.37-7.14 (m, 5H), 6.14-5.93 (m, 1H), 4.67-4.60 (m, 1H), 4.59-4.49 (m, 2H), 4.39-4.22 (m, 2H), 4.06 (s, 5H), 4.00-3.78 (m, 4H), 2.24 (d, J = 6.8 Hz, 1H), 2.05-1.98 (m, 3H), 1.87 (d, J = 15.6 Hz, 3H), 1.79 (d, J = 6.0 Hz, 2H), 1.65- 1.49 (m, 3H), 1.23- 1.17 (m, 2H), 1.08 (d, J = 7.2 Hz, 1H), 0.88-0.82 (m, 3H) |
| 263 | propyl ((fluoro(2- (((3S,6S,8S,9aR)-3- (3-(4-fluoropyridin- 3-yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 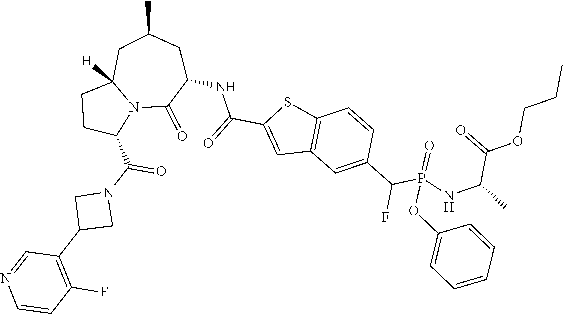 [SMILES: CCCOC(=O)[C@H](C)NP(=O)(Oc1ccccc1)C(F)c1ccc2sc(C(=O)N[C@H]3C[C@H](C)C[C@H]4CC[C@@H](C(=O)N5CC(c6cnccc6F)C5)N4C3=O)cc2c1] | 822.2 | ¹H NMR (400 MHz, CDCl₃) δ 9.14-8.98 (m, 1H), 8.78-8.61 (m, 1H), 8.03-7.95 (m, 1H), 7.92-7.85 (m, 1H), 7.83-7.74 (m, 1H), 7.62-7.47 (m, 2H), 7.41-7.31 (m, 2H), 7.30-7.28 (m, 1H), 7.25-7.18 (m, 2H), 7.13-7.05 (m, 1H), 6.09-5.87 (m, 1H), 5.07-4.85 (m, 2H), 4.73-4.61 (m, 1H), 4.55-4.45 (m, 1H), 4.40-4.26 (m, 1H), 4.22-4.12 (m, 3H), 4.10-3.97 (m, 2H), 3.76-3.56 (m, 1H), 2.37-2.22 (m, 2H), 2.16-2.03 (m, 4H), 2.02-1.91 (m, 2H), 1.68-1.51 (m, 3H), 1.34-1.26 (m, 5H), 1.13 (m, 1H), 0.95-0.83 (m, 3H |
| 264 | propyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)glycinate | 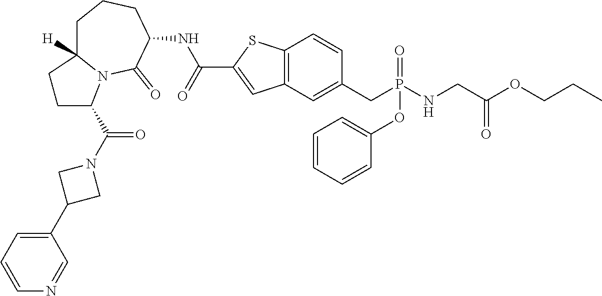 [SMILES: CCCOC(=O)CNP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 758.3 | ¹H NMR (400 MHz, CDCl₃) δ 8.49-8.67 (m, 2H) 7.85-7.95 (m, 1H) 7.75-7.84 (m, 2H) 7.61-7.75 (m, 2H) 7.47 (m, 1H) 7.29-7.41 (m, 2H) 7.14 (m, 3H) 4.41- 4.71 (m, 4H) 4.19 (m, 1H) 3.99-4.07 (m, 2H) 3.88-3.98 (m, 2H) 3.69-3.78 (m, 1H) 3.44-3.60 (m, 3H) 3.04-3.16 (m, 1H) 2.14-2.32 (m, 2H) 2.05 (s, 4H) 1.90-1.98 (m, 2H) 1.79-1.84 (m, 1H) 1.55-1.63 (m, 3H) 0.88 (m, 3H) |
| 265 | propyl (((2- (((3S,6S,10aS)-3- (((3H-imidazo[4,5- b]pyridin-6- yl)methyl) carbamoyl)-5- oxodecahydropyrrolo [1,2-a]azocin-6- yl)carbamoyl)benzo [b]thiophen-5- yl)fluoromethyl) (phenoxy)phosphoryl)- L-alaninate | 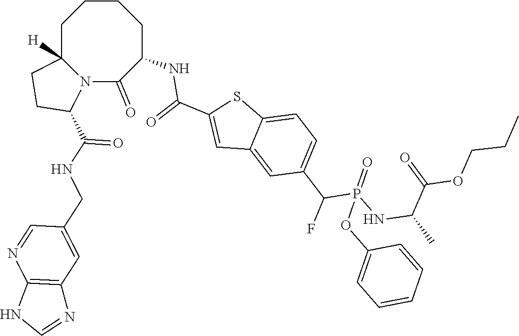 [SMILES: CCCOC(=O)[C@H](C)NP(=O)(Oc1ccccc1)C(F)c1ccc2sc(C(=O)N[C@H]3CCCC[C@H]4CC[C@@H](C(=O)NCc5cnc6[nH]cnc6c5)N4C3=O)cc2c1] | 818.0 | ¹H NMR (400 MHz, CD₃OD) δ 8.99 (s, 1H), 8.61 (d, J = 1.6 Hz, 1H), 8.33 (s, 1H), 8.15-8.04 (m, 2H), 8.02-7.94 (m, 1H), 7.64 (t, J = 7.2 Hz, 1H), 7.39-7.29 (m, 2H), 7.27-7.13 (m, 3H), 6.19-6.01 (m, 1H), 5.04-4.99 (m, 1H), 4.82-4.76 (m, 1H), 4.52-4.40 (m, 3H), 4.00-3.95 (m, 1H), 3.95-3.86 (m, 1H), 3.85-3.78 (m, 1H), 2.33-2.19 (m, 2H), 2.13-2.05 (m, 1H), 2.02-1.90 (m, 5H), 1.88-1.74 (m, 2H), 1.71-1.65 (m, 1H), 1.64-1.46 (m, 3H), 1.22 (t, J = 7.6 Hz, 2H), 1.08 (d, J = 7.2 Hz, 1H), 0.92- 0.83 (m, 3H) |
| 266 | propyl (((2- (((3S,6S,9aS)-3-(3- (6-cyanopyridin-2- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 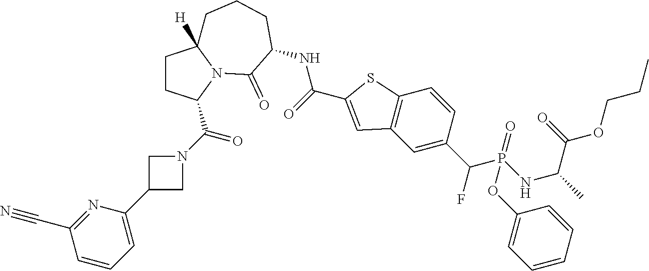 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cccc(C#N)n6)C5)N4C3=O)cc2c1] | 815.3 | ¹H NMR (400 MHz, DMSO-d₆) δ 8.80 (s, 1H), 8.28 (d, J = 11.4 Hz, 1H), 8.12-7.89 (m, 4H), 7.77-7.56 (m, 2H), 7.39-7.31 (m, 2H), 7.23-7.10 (m, 3H), 6.30-6.08 (m, 2H), 4.78-4.52 (m, 2H), 4.48-4.43 (m, 1H), 4.37-4.27 (m, 1H), 4.26-4.16 (m, 1H), 4.15-4.03 (m, 2H), 3.97 (d, J = 9.6 Hz, 3H), 3.76 (d, J = 7.6 Hz, 1H), 2.25- 2.15 (m, 1H), 2.08- 1.93 (m, 2H), 1.89- 1.65 (m, 7H), 1.53- 1.38 (m, 2H), 1.19- 0.90 (m, 3H), 0.86- 0.75 (m, 3H) |
| 267 | propyl (((2- (((3S,6S,9aS)-3-(3- (4- cyclopropylpyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 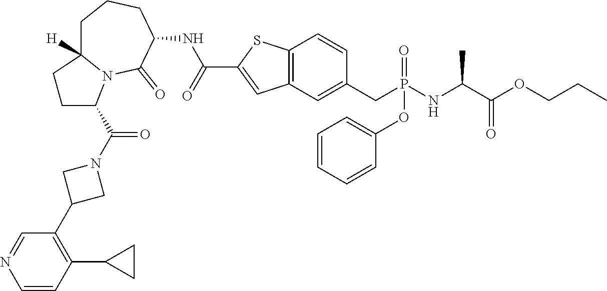 [SMILES: CCCOC(=O)[C@H](C)NP(=O)(Cc1ccc2sc(C(=O)N[C@H]3CCC[C@H]4CC[C@@H](C(=O)N5CC(c6cnccc6C6CC6)C5)N4C3=O)cc2c1)Oc1ccccc1] | 812.1 | ¹H NMR (400 MHz, CDCl₃) δ 8.65-8.56 (m, 1H), 8.45 (d, J = 4.0 Hz, 1H), 7.91- 7.77 (m, 3H), 7.67 (d, J = 4.0 Hz, 1H), 7.52-7.41 (m, 1H), 7.33-7.30 (m, 1H), 7.22-7.10 (m, 3H), 6.88-6.81 (m, 1H), 5.07-4.82 (m, 1H), 4.75-4.56 (m, 3H), 4.48-4.22 (m, 2H), 3.99-3.94 (m, 4H), 3.53-3.40 (m, 2H), 3.34-3.06 (m, 1H), 2.35-2.16 (m, 2H), 2.13-2.02 (m, 4H), 2.01-1.93 (m, 2H), 1.87-1.72 (m, 3H), 1.60-1.53 (m, 2H), 1.21-1.07 (m, 5H), 0.94-0.85 (m, 3H), 0.78 (d, J = 4.0 Hz, 2H) |
| 268 | propyl (((2- (((3S,6S,9aS)-3-(3- (4-chloropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 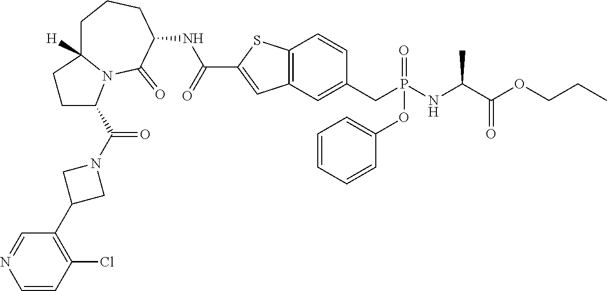 [SMILES: CCCOC(=O)[C@H](C)NP(=O)(Cc1ccc2sc(C(=O)N[C@H]3CCC[C@H]4CC[C@@H](C(=O)N5CC(c6cnccc6Cl)C5)N4C3=O)cc2c1)Oc1ccccc1] | 806.2 | ¹H NMR (400 MHz, CD₃OD) δ 8.78- 8.67 (m, 1H), 8.48- 8.42 (m, 1H), 8.06- 8.01 (m, 1H), 7.94- 7.84 (m, 2H), 7.61- 7.57 (m, 1H), 7.51- 7.46 (m, 1H), 7.35- 7.29 (m, 2H), 7.21- 7.12 (m, 3H), 4.74 (d, J = 8.4 Hz, 2H), 4.62-4.54 (m, 1H), 4.53-4.42 (m, 1H), 4.38-4.29 (m, 1H), 4.25-4.18 (m, 1H), 4.11-4.03 (m, 1H), 3.95-3.79 (m, 3H), 3.58-3.43 (m, 3H), 2.35-2.27 (m, 1H), 2.21-2.07 (m, 2H), 2.06-1.96 (m, 3H), 1.95-1.88 (m, 2H), 1.86-1.78 (m, 2H), 1.57-1.48 (m, 2H), 1.33-1.28 (m, 2H), 1.14 (d, J = 7.2 Hz, 1H), 0.89-0.83 (m, 3H) |
| 269 | propyl (((2- (((4S,7S,11aS)-4- ((rel-trans)-3- cyano-4- phenylpyrrolidine- 1-carbonyl)-6- oxodecahydro-2H- pyrido[1,2- a]azocin-7- yl)carbamoyl)benzo [b]thiophen-5- yl)fluoromethyl) (phenoxy)phosphoryl)- L-alaninate | 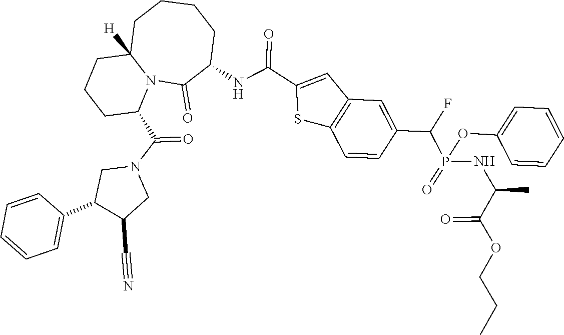 | 856.2 | ¹H NMR (400 MHz, CDCl₃) δ 7.98 (d, J = 9.6 Hz, 1H), 7.91- 7.78 (m, 2H), 7.62- 7.52 (m, 1H), 7.47 (d, J = 7.6 Hz, 1H), 7.44-7.30 (m, 6H), 7.28 (s, 1H), 7.25- 7.18 (m, 1H), 7.14- 7.05 (m, 1H), 6.08- 5.86 (m, 1H), 5.20- 5.06 (m, 1H), 4.91 (s, 1H), 4.41-4.20 (m, 2H), 4.12-3.99 (m, 3H), 3.88-3.49 (m, 4H), 3.42-3.14 (m, 1H), 2.65-2.54 (m, 1H), 2.26-2.20 (m, 1H), 2.03-1.91 (m, 4H), 1.90-1.80 (m, 2H), 1.77-1.71 (m, 1H), 1.62 ( J = 7.2, 14.6 Hz, 3H), 1.56-1.49 (m, 2H), 1.46-1.40 (m, 1H), 1.32-1.28 (m, 2H), 1.26 (s, 1H), 1.17- 1.10 (m, 1H), 0.95- 0.84 (m, 3H) |
| 270 | propyl (((2- (((1R,3S,6S,11aS)- 3-((rel-trans)-3- cyano-4- phenylpyrrolidine- 1-carbonyl)-1- hydroxy-5- oxodecahydro-1H- pyrrolo[1,2- a]azonin-6- yl)carbamoyl)benzo [b]thiophen-5- yl)fluoromethyl) (phenoxy)phosphoryl)- L-alaninate | 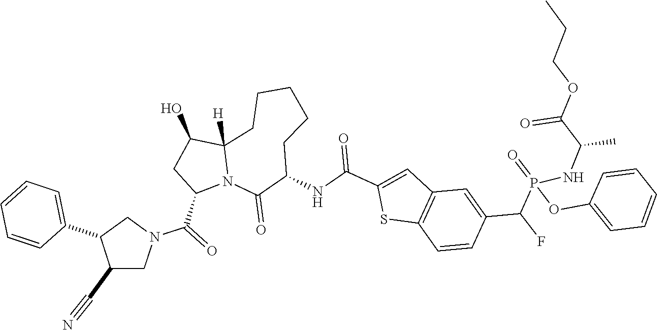 | 872.4 | ¹H NMR (400 MHz, CD₃OD) δ 8.14- 8.05 (m, 2H), 8.01- 7.91 (m, 1H), 7.68- 7.58 (m, 1H), 7.44- 7.27 (m, 7H), 7.24- 7.11 (m, 3H), 6.18- 6.00 (m, 1H), 5.15 (,m, 1H), 4.95-4.89 (m, 1H), 4.22-4.08 (m, 3H), 4.07-3.70 (m, 5H), 3.68-3.37 (m, 3H), 2.30-2.20 (m, 1H), 2.16-2.02 (m, 3H), 2.00-1.89 (m, 2H), 1.85-1.72 (m, 3H), 1.68-1.47 (m, 5H), 1.24-1.19 (m, 2H), 1.05 (d, J = 7.2 Hz, 1H), 0.91- 0.82 (m, 3H) |
| 271 | propyl (((2- (((3S,6S,9aS)-3-(3- (4-fluoro-6- methoxypyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 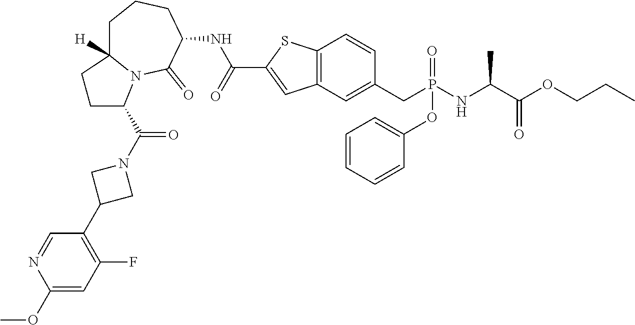 [SMILES: CCCOC(=O)[C@H](C)NP(=O)(Cc1ccc2sc(C(=O)N[C@H]3CCC[C@H]4CC[C@@H](C(=O)N5CC(c6cnc(OC)cc6F)C5)N4C3=O)cc2c1)Oc1ccccc1] | 820.4 | ¹H NMR (400 MHz, CD₃OD) δ 8.23- 7.90 (m, 2H), 7.88- 7.70 (m, 2H), 7.59- 6.95 (m, 6H), 6.64- 6.40 (m, 1H), 4.67- 4.45 (m, 3H), 4.37- 4.24 (m, 1H), 4.12- 3.69 (m, 9H), 3.54- 3.33 (m, 3H), 2.27- 2.15 (m, 1H), 2.10- 1.65 (m, 9H), 1.55- 1.35 (m, 2H), 1.16- 0.92 (m, 3H), 0.89- 0.68 (m, 3H) |
| 272 | propyl (((2- (((3S,6S,9aS)-3-(3- (2- (difluoromethyl) pyridin-3-yl)azetidine- 1-carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 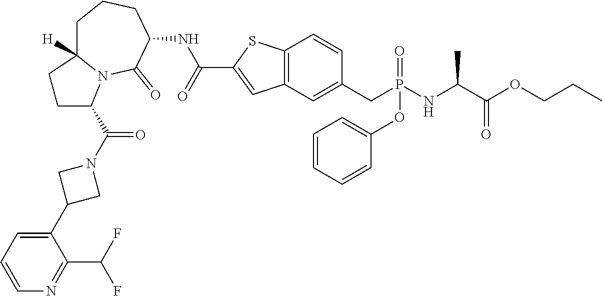 [SMILES: CCCOC(=O)[C@H](C)NP(=O)(Cc1ccc2sc(C(=O)N[C@H]3CCC[C@H]4CC[C@@H](C(=O)N5CC(c6cccnc6C(F)F)C5)N4C3=O)cc2c1)Oc1ccccc1] | 822.6 | ¹H NMR (400 MHz, CDCl₃) δ 8.56 (s, 1H), 8.31-7.99 (m, 1H), 7.91-7.74 (m, 3H), 7.71-7.62 (m, 1H), 7.58-7.51 (m, 1H), 7.49-7.39 (m, 1H), 7.31-7.29 (m, 1H), 7.21-7.08 (m, 3H), 6.87-6.53 (m, 1H), 4.71-4.67 (m, 1H), 4.64-4.53 (m, 3H), 4.45-4.10 (m, 2H), 4.07-3.86 (m, 4H), 3.53-3.39 (m, 2H), 3.38-3.09 (m, 1H), 2.27-2.18 (m, 2H), 2.11-2.00 (m, 4H), 1.99-1.90 (m, 2H), 1.87-1.67 (m, 2H), 1.64-1.49 (m, 2H), 1.17-1.13 (m, 3H), 0.95-0.80 (m, 3H) |
| 273 | propyl (((2- (((3S,6S,9aS)-3-(3- (4-cyanopyridin-3- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 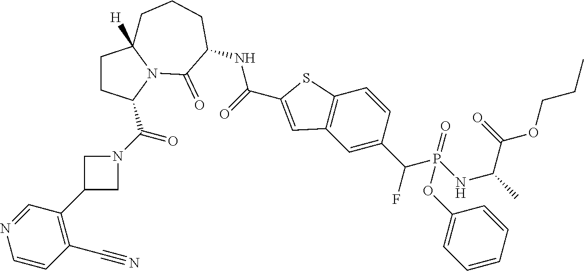 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cnccc6C#N)C5)N4C3=O)cc2c1] | 815.1 | ¹H NMR (400 MHz, CDCl₃) δ 8.97-8.63 (m, 1H), 8.52-8.42 (m, 1H), 7.75-7.67 (m, 1H), 7.63-7.51 (m, 2H), 7.43-7.20 (m, 3H), 7.09-7.03 (m, 1H), 6.94-6.88 (m, 1H), 6.86-6.69 (m, 2H), 5.81-5.55 (m, 1H), 4.55-4.45 (m, 1H), 4.41 (t, J = 10.0 Hz, 1H), 4.28- 4.20 (m, 1H), 4.12- 3.99 (m, 2H), 3.99- 3.82 (m, 1H), 3.81- 3.71 (m, 2H), 3.70- 3.54 (m, 2H), 3.42- 3.24 (m, 1H), 2.02- 1.87 (m, 2H), 1.77 (s, 4H), 1.63 (d, J = 11.6 Hz, 2H), 1.37-1.21 (m, 3H), 1.05-0.91 (m, 3H), 0.82 (t, J = 6.4 Hz, 1H), 0.65- 0.54 (m, 3H) |
| 274 | propyl (((2- (((3S,6S,10aS)-3- (3-(4-fluoropyridin- 3-yl)azetidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 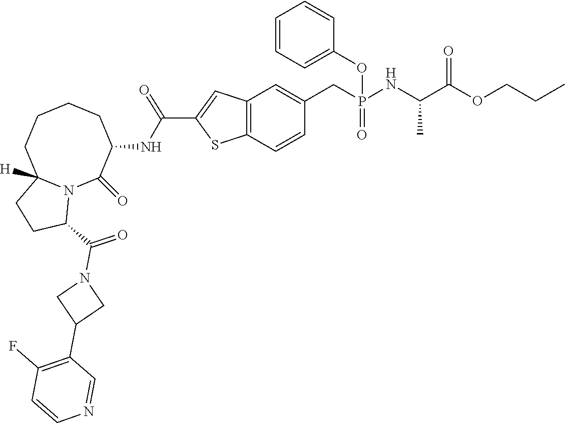 [SMILES: CCCOC(=O)[C@H](C)NP(=O)(Cc1ccc2sc(C(=O)N[C@H]3CCCC[C@H]4CC[C@@H](C(=O)N5CC(c6cnccc6F)C5)N4C3=O)cc2c1)Oc1ccccc1] | 804.4 | ¹H NMR (400 MHz, CD₃OD) δ 9.00- 8.58 (m, 2H), 8.10- 7.84 (m, 3H), 7.57- 7.45 (m, 2H), 7.37- 7.28 (m, 2H), 7.26- 7.11 (m, 3H), 5.04- 4.99 (m, 2H), 4.77- 4.70 (m, 1H), 4.58- 4.38 (m, 4H), 4.32- 4.12 (m, 2H), 3.94- 3.86 (m, 2H), 3.58- 3.49 (m, 2H), 2.31- 2.20 (m, 2H), 2.08- 1.94 (m, 6H), 1.90- 1.77 (m, 2H), 1.75- 1.61 (m, 2H), 1.59- 1.48 (m, 2H), 1.16- 1.06 (m, 3H), 0.92- 0.83 (m, 3H) |
| 275 | propyl (((2- (((3S,6S,9aS)-3-(3- (4-fluoro-2- methoxypyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 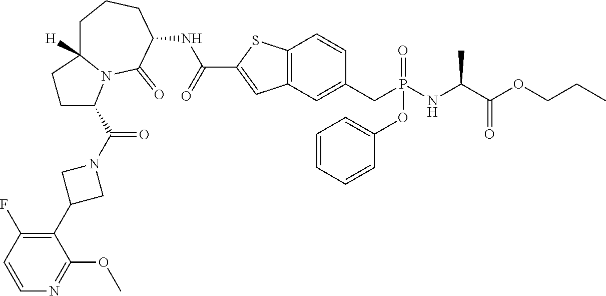 [SMILES: CCCOC(=O)[C@H](C)NP(=O)(Cc1ccc2sc(C(=O)N[C@H]3CCC[C@H]4CC[C@@H](C(=O)N5CC(c6c(F)ccnc6OC)C5)N4C3=O)cc2c1)Oc1ccccc1] | 820.4 | ¹H NMR (400 MHz, CD₃OD) δ 8.07- 8.00 (m, 2H), 7.88- 7.86 (m, 2H), 7.50- 7.45 (m, 1H), 7.31- 7.29 (m, 2H), 7.19- 7.13 (m, 3H), 6.80- 6.79 (m, 1H), 4.76- 4.72 (m, 2H), 4.60- 4.58 (m, 2H), 4.28- 4.27 (m, 3H), 4.09- 4.04 (m, 1H), 4.00 (d, J = 6.0 Hz, 3H), 3.93-3.90 (m, 3H), 3.56-3.50 (m, 2H), 2.33-2.28 (m, 1H), 2.13-1.80 (m, 9H), 1.58-1.51 (m, 2H), 1.14-1.06 (m, 3H), 0.88-0.84 (m, 3H) |
| 276 | (S)-sec-butyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 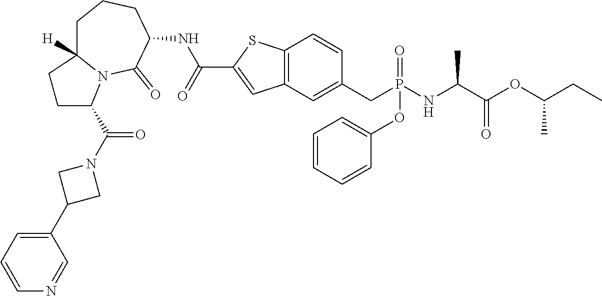 [SMILES: CC[C@H](C)OC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 786.3 | ¹H NMR (400 MHz, CDCl₃) δ 9.04-8.81 (m, 1H), 8.78-8.67 (m, 1H), 8.52-8.10 (m, 1H), 7.86 (s, 1H), 7.85-7.77 (m, 3H), 7.77-7.60 (m, 1H), 7.48-7.37 (m, 1H), 7.32-7.28 (m, 1H), 7.20-7.07 (m, 3H), 4.80-4.66 (m, 4H), 4.58-4.42 (m, 2H), 4.28-4.13 (m, 1H), 4.10-3.92 (m, 3H), 3.53-3.40 (m, 2H), 2.35-2.24 (m, 1H), 2.24-2.13 (m, 1H), 2.12-2.00 (m, 4H), 1.98-1.68 (m, 4H), 1.59-1.42 (m, 2H), 1.24-1.09 (m, 6H), 0.88-0.78 (m, 3H) |
| 277 | isobutyl ((fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 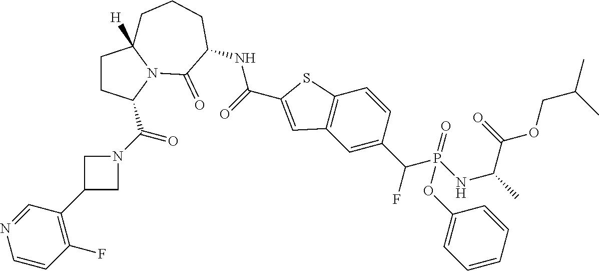 [SMILES: CC(C)COC(=O)[C@H](C)NP(=O)(Oc1ccccc1)C(F)c1ccc2sc(C(=O)N[C@H]3CCC[C@H]4CC[C@@H](C(=O)N5CC(c6cnccc6F)C5)N4C3=O)cc2c1] | 822.2 | ¹H NMR (400 MHz, CDCl₃) δ 8.80-8.51 (m, 2H), 7.98 (d, J = 8.8 Hz, 1H), 7.92- 7.78 (m, 2H), 7.73- 7.65 (m, 1H), 7.62- 7.52 (m, 1H), 7.40- 7.32 (m, 1H), 7.27- 7.14 (m, 2H), 7.11- 7.04 (m, 2H), 6.08- 5.85 (m, 1H), 5.02- 4.74 (m, 1H), 4.71- 4.55 (m, 3H), 4.39- 4.32 (m, 1H), 4.24- 4.09 (m, 2H), 3.97- 3.86 (m, 2H), 3.86- 3.77 (m, 1H), 3.72- 3.51 (m, 1H), 2.32- 2.26 (m, 1H), 2.25- 2.18 (m, 1H), 2.10- 2.01 (m, 4H), 1.99- 1.88 (m, 3H), 1.85- 1.80 (m, 1H), 1.38- 1.22 (m, 3H), 1.17- 1.07 (m, 1H), 0.95- 0.80 (m, 6H) |
| 278 | propyl (((2- (((3S,6S,9aS)-3-(3- (6-fluoro-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 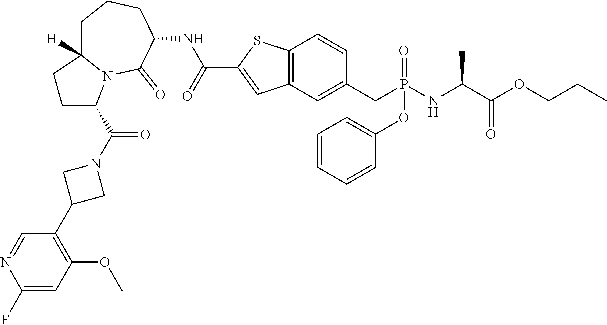 [SMILES: CCCOC(=O)[C@H](C)NP(=O)(Cc1ccc2sc(C(=O)N[C@H]3CCC[C@H]4CC[C@@H](C(=O)N5CC(c6cnc(F)cc6OC)C5)N4C3=O)cc2c1)Oc1ccccc1] | 820.4 | ¹H NMR (400 MHz, CD₃OD) δ 8.05- 7.97 (m, 2H), 8.06- 7.84 (m, 2H), 7.53- 7.45 (m, 1H), 7.35- 7.28 (m, 2H), 7.21- 7.10 (m, 3H), 6.72 (d, J = 16.6 Hz, 1H), 4.75-4.71 (m, 1H), 4.59-4.56 (m, 2H), 4.42-4.36 (m, 1H), 4.36-4.26 (m, 1H), 4.19-4.09 (m, 1H), 4.08-4.00 (m, 2H), 3.99-3.95 (m, 3H), 3.95-3.79 (m, 3H), 3.57-3.46 (m, 2H), 2.36-2.25 (m, 1H), 2.13-1.82 (m, 9H), 1.57-1.48 (m, 2H), 1.16-1.04 (m, 3H), 0.90-0.83 (m, 3H) |
| 279 | butyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 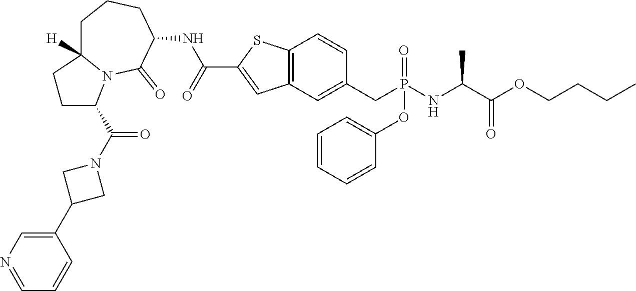 [SMILES: CCCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 786.4 | ¹H NMR (400 MHz, CDCl₃) δ 8.95-8.62 (m, 2H), 8.51 (d, J = 6.8 Hz, 1H), 7.88- 7.74 (m, 4H), 7.49- 7.35 (m, 1H), 7.29 (s, 2H), 7.18-7.02 (m, 3H), 4.81-4.37 (m, 5H), 4.28-3.89 (m, 6H), 3.71-3.36 (m, 3H), 2.33-2.10 (m, 3H), 2.06 (d, J = 4.0 Hz, 3H), 1.98-1.69 (m, 4H), 1.57-1.47 (m, 2H), 1.37-1.26 (m, 2H), 1.20-1.14 (m, 2H), 0.97-0.80 (m, 3H) |
| 280 | propyl (((2- (((3S,6S,10aS)-3- (((3H-imidazo[4,5- b]pyridin-6- yl)methyl) carbamoyl)-5- oxodecahydropyrrolo [1,2-a]azocin-6- yl)carbamoyl)benzo [b]thiophen-5- yl)methyl)(phenoxy) phosphoryl)-L- alaninate | 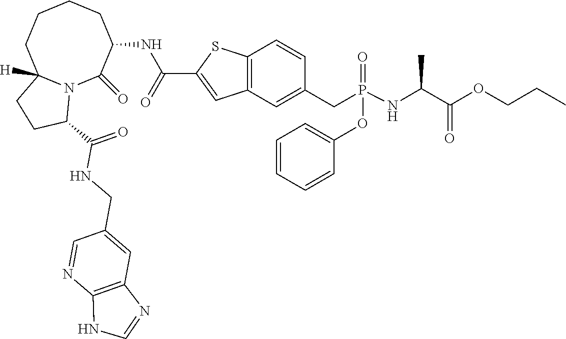 [SMILES: CCCOC(=O)[C@H](C)NP(=O)(Cc1ccc2sc(C(=O)N[C@H]3CCCC[C@H]4CC[C@@H](C(=O)NCc5cnc6[nH]cnc6c5)N4C3=O)cc2c1)Oc1ccccc1] | 800.4 | ¹H NMR (400 MHz, CDCl₃) δ 8.46-8.35 (m, 1H), 8.30-8.11 (m, 2H), 8.02 (d, J = 15.2 Hz, 1H), 7.83 (d, J = 2.4 Hz, 1H), 7.71 (d, J = 8.8 Hz, 2H), 7.31-7.28 (m, 1H), 7.26-7.23 (m, 1H), 7.18-7.06 (m, 3H), 5.28-5.07 (m, 1H), 4.96-4.73 (m, 1H), 4.51 (s, 1H), 4.40-4.30 (m, 1H), 4.25-4.11 (m, 1H), 4.01-3.87 (m, 3H), 3.50-3.30 (m, 2H), 2.29-1.96 (m, 7H), 1.81-1.70 (m, 2H), 1.68-1.44 (m, 5H), 1.17-1.12 (m, 3H), 0.89-0.78 (m, 3H) |
| 281 | isobutyl (((2- (((3S,6S,10aS)-3- (3-methoxy-3- (pyridin-3- yl)azetidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 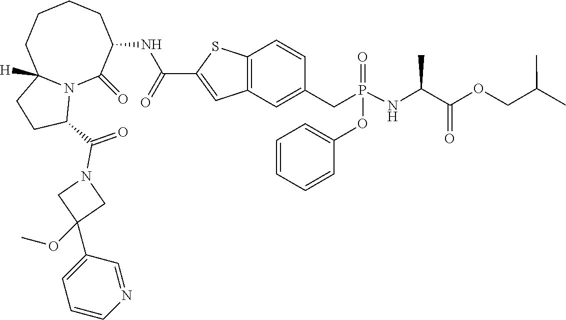 [SMILES: COC1(c2cccnc2)CN(C(=O)[C@@H]2CC[C@@H]3CCCC[C@H](NC(=O)c4cc5cc(CP(=O)(N[C@@H](C)C(=O)OCC(C)C)Oc6ccccc6)ccc5s4)C(=O)N32)C1] | 830.1 | ¹H NMR (400 MHz, CDCl₃) δ 9.65 (s, 1H), 8.87-8.63 (m, 1H), 8.48 (d, J = 8.4 Hz, 1H), 8.00 (s, 1H), 7.95-7.75 (m, 4H), 7.45-7.37 (m, 1H), 7.34-7.28 (m, 2H), 7.18-7.09 (m, 3H), 5.21-5.00 (m, 2H), 4.69-4.50 (m, 1H), 4.43-4.30 (m, 3H), 4.27-4.20 (m, 1H), 4.04-3.92 (m, 1H), 3.87-3.72 (m, 2H), 3.53-3.44 (m, 2H), 3.38 (s, 3H), 2.29-2.14 (m, 2H), 2.11-1.93 (m, 4H), 1.90-1.73 (m, 4H), 1.71-1.59 (m, 2H), 1.23-1.12 (m, 3H), 0.93-0.82 (m, 6H) |
| 282 | isobutyl (((2- (((3S,6S,10aS)-3- (3-(4- methylpyridin-3- yl)azetidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 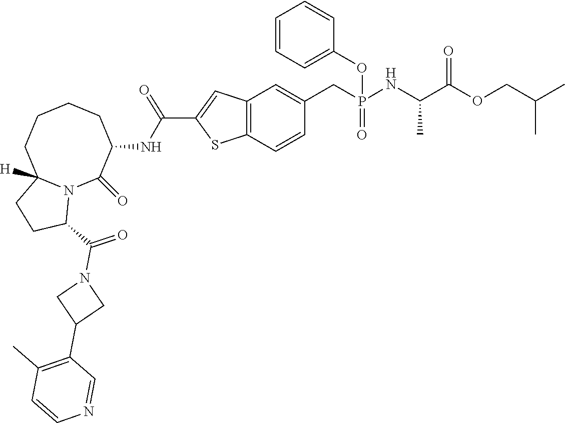 [SMILES: Cc1ccncc1C1CN(C(=O)[C@@H]2CC[C@@H]3CCCC[C@H](NC(=O)c4cc5cc(CP(=O)(N[C@@H](C)C(=O)OCC(C)C)Oc6ccccc6)ccc5s4)C(=O)N32)C1] | 814.2 | ¹H NMR (400 MHz, CDCl₃) δ 9.12-8.44 (m, 1H), 8.35-8.15 (m, 1H), 7.88-7.65 (m, 4H), 7.49-7.30 (m, 3H), 7.14 ( d, J = 7.6 Hz, 3H), 5.13- 4.92 (m, 1H), 4.52 (s, 2H), 4.45-4.27 (m, 3H), 4.13-3.94 (m, 3H), 3.87-3.72 (m, 2H), 3.66-3.56 (m, 1H), 3.54-3.38 (m, 2H), 2.37-2.25 (m, 3H), 2.18 ( d, J = 5.6 Hz, 2H), 2.13- 2.02 (m, 2H), 1.97 (d, J = 3.6 Hz, 2H), 2.01-1.71 (m, 6H), 1.69-1.50 (m, 2H), 1.29-1.16 (m, 3H), 0.95-0.82 (m, 6H) |
| 283 | propyl (((2- (((3S,6S,8S,9aR)-8- methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 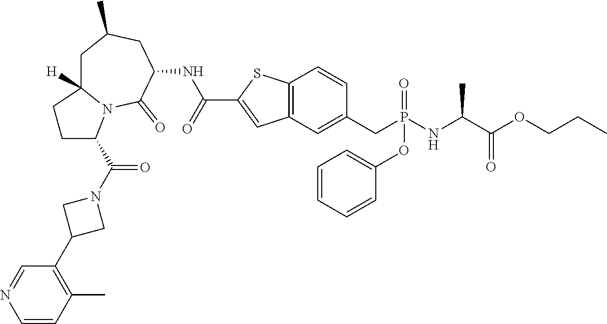 [SMILES: CCCOC(=O)[C@H](C)NP(=O)(Cc1ccc2sc(C(=O)N[C@H]3C[C@H](C)C[C@H]4CC[C@@H](C(=O)N5CC(c6cnccc6C)C5)N4C3=O)cc2c1)Oc1ccccc1] | 800.4 | ¹H NMR (400 MHz, CDCl₃) δ 8.67-8.55 (m, 1H), 8.15-7.35 (m, 6H), 7.32-7.27 (m, 2H), 7.20-7.08 (m, 3H), 5.17-4.90 (m, 2H), 4.77-4.63 (m, 1H), 4.55-4.38 (m, 2H), 4.27-4.10 (m, 2H), 4.08-3.89 (m, 4H), 3.51-3.38 (m, 2H), 2.47 (s, 3H), 2.36-2.19 (m, 2H), 2.18-1.88 (m, 6H), 1.67-1.51 (m, 3H), 1.35-1.14 (m, 6H), 0.87 (q, J = 7.2 Hz, 3H) |
| 284 | propyl (((2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 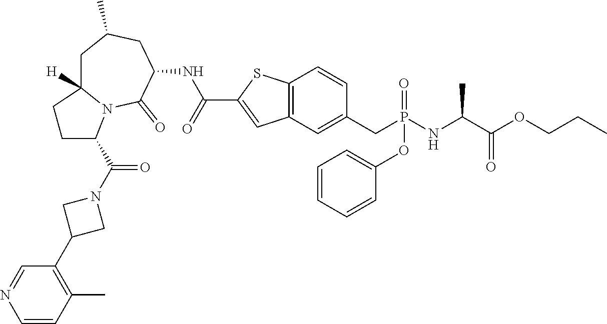 [SMILES: CCCOC(=O)[C@H](C)NP(=O)(Cc1ccc2sc(C(=O)N[C@H]3C[C@@H](C)C[C@H]4CC[C@@H](C(=O)N5CC(c6cnccc6C)C5)N4C3=O)cc2c1)Oc1ccccc1] | 800.3 | ¹H NMR (400 MHz, CDCl₃) δ 9.24-8.87 (m, 1H), 8.71-8.57 (m, 1H), 7.98-7.75 (m, 3H), 7.62 (m, 1H), 7.57-7.36 (m, 2H), 7.32-7.27 (m, 2H), 7.17-7.10 (m, 3H), 5.13-5.02 (m, 1H), 4.86-4.64 (m, 2H), 4.58-4.40 (m, 2H), 4.15-3.83 (m, 6H), 3.51-3.39 (m, 2H), 2.49 (s, 3H), 2.34-1.98 (m, 6H), 1.84-1.46 (m, 5H), 1.22-1.12 (m, 3H), 1.06-0.98 (m, 3H), 0.91-0.83 (m, 3H) |
| 285 | butyl N- ((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-N- methyl-L-alaninate | 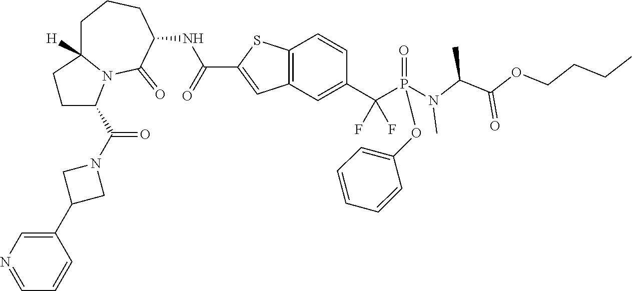 [SMILES: CCCCOC(=O)[C@H](C)N(C)P(=O)(Oc1ccccc1)C(F)(F)c1ccc2sc(C(=O)N[C@H]3CCC[C@H]4CC[C@@H](C(=O)N5CC(c6cccnc6)C5)N4C3=O)cc2c1] | 836.4 | ¹H NMR (400 MHz, CDCl₃) δ 9.13-8.83 (m, 1H), 8.80-8.68 (m, 1H), 8.58-8.11 (m, 2H), 8.03-7.89 (m, 2H), 7.88-7.75 (m, 2H), 7.74 (s, 1H), 7.32-7.29 (m, 1H), 7.22-7.09 (m, 3H), 4.89-4.66 (m, 3H), 4.64-4.48 (m, 3H), 4.20-4.15 (m, 1H), 4.07 (t, J = 6.4 Hz, 2H), 3.99 (t, J = 6.4 Hz, 2H), 2.88-2.76 (m, 3H), 2.17 (d, J = 14.0 Hz, 2H), 2.13- 1.99 (m, 4H), 1.98- 1.71 (m, 4H), 1.60- 1.48 (m, 2H), 1.40 (d, J = 7.2 Hz, 1H), 1.33 (t, J = 7.2 Hz, 4H), 0.96-0.82 (m, 3H) |
| 286 | propyl (((2- (((3S,6S,9S,10aR)- 9-methyl-3-(3-(4- methylpyridin-3- yl)azetidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 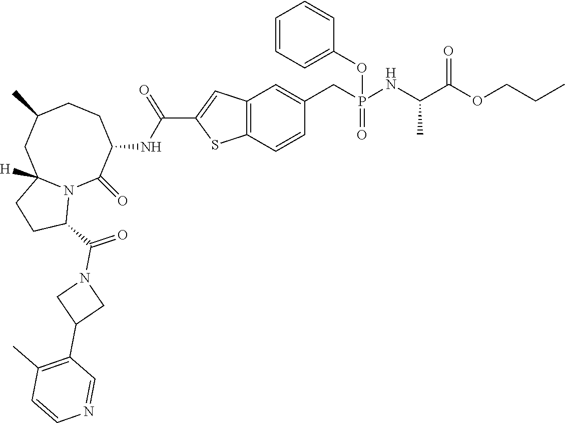 [SMILES: CCCOC(=O)[C@H](C)NP(=O)(Cc1ccc2sc(C(=O)N[C@H]3CC[C@H](C)C[C@H]4CC[C@@H](C(=O)N5CC(c6cnccc6C)C5)N4C3=O)cc2c1)Oc1ccccc1] | 814.6 | ¹H NMR (400 MHz, CDCl₃) δ 9.53 (s, 1H), 8.69-8.53 (m, 1H), 8.23-8.07 (m, 1H), 7.99 (d, J = 6.0 Hz, 1H), 7.87-7.79 (m, 1H), 7.78-7.74 (m, 1H), 7.56 (dd, J = 5.2, 18.0 Hz, 1H), 7.46-7.34 (m, 1H), 7.32-7.28 (m, 2H), 7.16-7.11 (m, 3H), 5.03 (d, J = 4.0 Hz, 1H), 4.97 (d, J = 9.2 Hz, 1H), 4.74-4.67 (m, 1H), 4.49-4.43 (m, 1H), 4.37 (t, J = 8.0 Hz, 2H), 4.24- 4.08 (m, 1H), 4.07- 3.98 (m, 2H), 3.97- 3.93 (m, 2H), 3.51- 3.41 (m, 2H), 2.48 (d, J = 12.8 Hz, 3H), 2.32-2.18 (m, 2H), 2.16-2.08 (m, 2H), 2.06-1.90 (m, 2H), 1.88-1.80 (m, 2H), 1.75-1.65 (m, 2H), 1.61-1.53 (m, 2H), 1.37-1.29 (m, 1H), 1.18 (dd, J = 7.2, 16.4 Hz, 3H), 1.04 (d, J = 6.8 Hz, 3H), 0.91- 0.85 (m, 3H) |
| 287 | propyl (((2- (((3S,6S,9R,10aR)- 9-methyl-3-(3-(4- methylpyridin-3- yl)azetidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 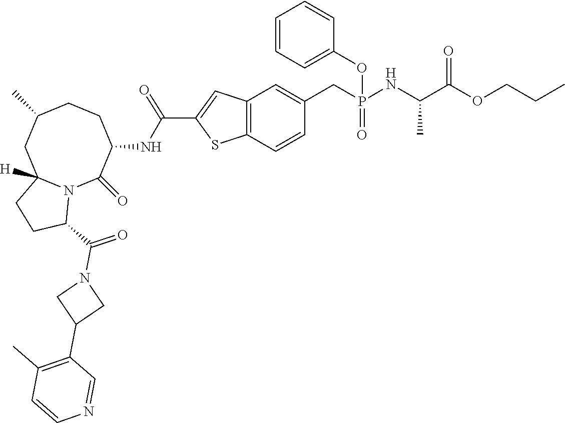 [SMILES: CCCOC(=O)[C@H](C)NP(=O)(Cc1ccc2sc(C(=O)N[C@H]3CC[C@@H](C)C[C@H]4CC[C@@H](C(=O)N5CC(c6cnccc6C)C5)N4C3=O)cc2c1)Oc1ccccc1] | 814.1 | ¹H NMR (400 MHz, CDCl₃) δ 9.66 (s, 1H), 8.67-8.50 (m, 1H), 8.23-8.14 (m, 1H), 8.05 (d, J = 6.4 Hz, 1H), 7.85-7.74 (m, 2H), 7.52 (s, 1H), 7.47-7.36 (m, 1H), 7.35-7.27 (m, 2H), 7.17-7.10 (m, 3H), 5.27-5.15 (m, 1H), 5.13-4.98 (m, 1H), 4.74-4.61 (m, 1H), 4.47-4.34 (m, 3H), 4.21-4.06 (m, 1H), 4.06-3.98 (m, 2H), 3.98-3.92 (m, 2H), 3.50-3.39 (m, 2H), 2.51-2.38 (m, 3H), 2.25-2.17 (m, 2H), 2.15-2.10 (m, 1H), 2.06-1.98 (m, 2H), 1.93 (d, J = 11.6 Hz, 1H), 1.89-1.80 (m, 3H), 1.62-1.53 (m, 3H), 1.45 (d, J = 14.0 Hz, 1H), 1.18 (dd, J = 7.2, 19.2 Hz, 3H), 1.03 (d, J = 6.0 Hz, 3H), 0.92-0.84 (m, 3H) |
| 288 | butyl (((2- (((3S,6S,9aS)-3-(3- (3-methylpyridin-2- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 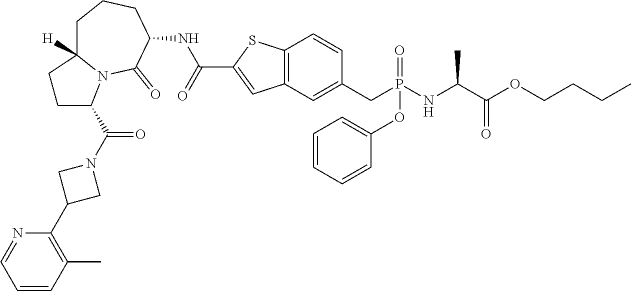 [SMILES: CCCCOC(=O)[C@H](C)NP(=O)(Cc1ccc2sc(C(=O)N[C@H]3CCC[C@H]4CC[C@@H](C(=O)N5CC(c6ncccc6C)C5)N4C3=O)cc2c1)Oc1ccccc1] | 800.0 | ¹H NMR (400 MHz, CD₃OD) δ 8.55- 8.40 (m, 1H), 8.17- 7.97 (m, 2H), 7.93- 7.84 (m, 2H), 7.59- 7.46 (m, 2H), 7.35- 7.28 (m, 2H), 7.21- 7.15 (m, 2H), 7.15- 7.01 (m, 1H), 4.77- 4.71 (m, 2H), 4.61- 4.41 (m, 4H), 4.35- 4.24 (m, 1H), 4.13- 4.04 (m, 1H), 4.03- 3.90 (m, 2H), 3.89- 3.78 (m, 1H), 3.59- 3.44 (m, 2H), 2.40 (d, J = 16.0 Hz, 3H), 2.35-2.26 (m, 1H), 2.21-2.06 (m, 2H), 2.02 (dd, J = 5.6, 12.0 Hz, 2H), 1.97 (d, J = 4.8 Hz, 2H), 1.89- 1.79 (m, 3H), 1.55- 1.45 (m, 2H), 1.34- 1.25 (m, 2H), 1.16- 1.06 (m, 3H), 0.94- 0.86 (m, 3H) |
| 289 | propyl (((2- (((3S,6S,9aS)-3-(3- (6-cyanopyridin-2- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 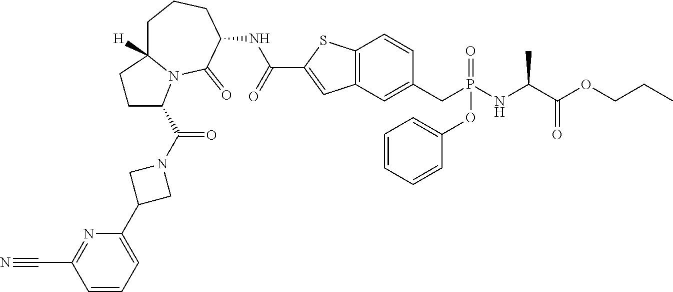 [SMILES: CCCOC(=O)[C@H](C)NP(=O)(Cc1ccc2sc(C(=O)N[C@H]3CCC[C@H]4CC[C@@H](C(=O)N5CC(c6cccc(C#N)n6)C5)N4C3=O)cc2c1)Oc1ccccc1] | 797.2 | ¹H NMR (400 MHz, CD₃OD) δ 8.05- 8.01 (m, 1H), 7.98- 7.83 (m, 3H), 7.79- 7.58 (m, 2H), 7.49 (s, 1H), 7.35-7.28 (m, 2H), 7.22-7.12 (m, 3H), 4.79-4.55 (m, 3H), 4.51-4.25 (m, 2H), 4.21-4.02 (m, 3H), 3.99-3.76 (m, 3H), 3.61-3.40 (m, 2H), 2.41-2.25 (m, 1H), 2.21-2.10 (m, 1H), 2.07-1.99 (m, 3H), 1.98 (s, 5H), 1.61-1.45 (m, 2H), 1.18-1.03 (m, 3H), 0.90-0.82 (m, 3H) |
| 290 | propyl (((2- (((3S,6S,9aS)-3-(3- (4-cyanopyrimidin- 2-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 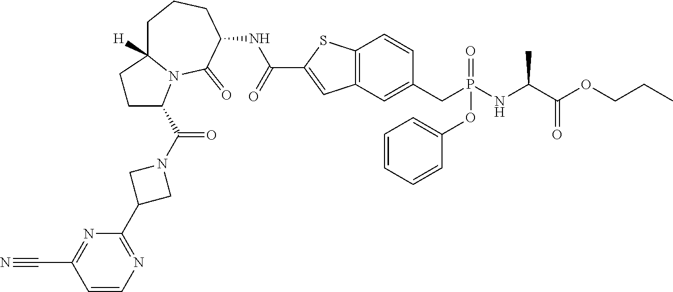 [SMILES: CCCOC(=O)[C@H](C)NP(=O)(Cc1ccc2sc(C(=O)N[C@H]3CCC[C@H]4CC[C@@H](C(=O)N5CC(c6nccc(C#N)n6)C5)N4C3=O)cc2c1)Oc1ccccc1] | 798.3 | ¹H NMR (400 MHz, CDCl₃) δ 8.92-9.03 (m, 1H) 7.64-7.90 (m, 4H) 7.51-7.60 (m, 1H) 7.35-7.48 (m, 1H) 7.27 (s, 2H) 7.09-7.17 (m, 2H) 4.82-5.01 (m, 1H) 4.69 (m, 1H) 4.59 (m, 2H) 4.35-4.52 (m, 2H) 4.22-4.27 (m, 1H) 3.87-4.07 (m, 4H) 3.40-3.53 (m, 2H) 2.15-2.33 (m, 2H) 1.87-2.12 (m, 6H) 1.66-1.85 (m, 2H) 1.48-1.64 (m, 2H) 1.08-1.24 (m, 3H) 0.78-0.97 (m, 3H) |
| 291 | 3,3,3- trifluoropropyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 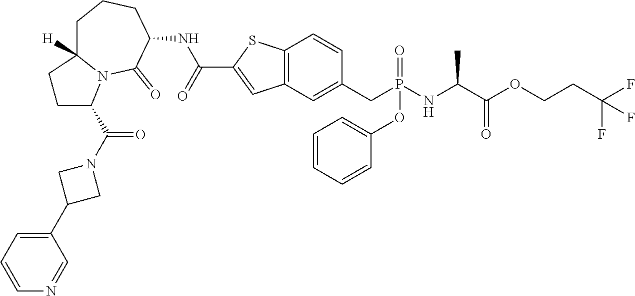 [SMILES: C[C@H](NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1)C(=O)OCCC(F)(F)F] | 826.5 | ¹H NMR (400 MHz, CDCl₃) δ 9.01-8.82 (m, 1H), 8.78-8.67 (m, 1H), 8.52 (s, 1H), 8.28-8.02 (m, 2H), 7.87-7.78 (m, 4H), 7.45-7.39 (m, 1H), 7.18-7.09 (m, 3H), 4.81-4.45 (m, 4H), 4.29-4.11 (m, 3H), 4.09-3.93 (m, 3H), 3.66-3.35 (m, 3H), 2.43-2.25 (m, 3H), 2.19-2.01 (m, 5H), 1.97-1.67 (m, 4H), 1.22-1.10 (m, 3H) |
| 292 | propyl ((fluoro(2- (((3S,6S,8R,9aR)- 3-(3-(4- fluoropyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 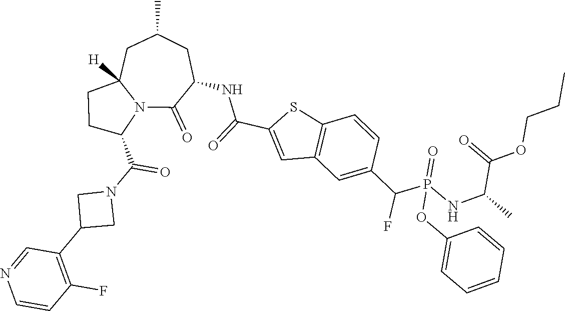 [SMILES: CCCOC(=O)[C@H](C)NP(=O)(Oc1ccccc1)C(F)c1ccc2sc(C(=O)N[C@H]3C[C@@H](C)C[C@H]4CC[C@@H](C(=O)N5CC(c6cnccc6F)C5)N4C3=O)cc2c1] | 822.2 | ¹H NMR (400 MHz, CDCl₃) δ 8.81-8.51 (m, 2H), 8.02-7.93 (m, 1H), 7.90-7.80 (m, 2H), 7.69 (s, 1H), 7.63-7.54 (m, 1H), 7.40-7.32 (m, 1H), 7.30-7.27 (m, 1H), 7.24 (d, J = 2.0 Hz, 1H), 7.15-7.06 (m, 3H), 6.08-5.84 (m, 1H), 4.70 (m, 1H), 4.66-4.54 (m, 2H), 4.42-4.30 (m, 1H), 4.23-4.09 (m, 2H), 4.06 (m, 2H), 4.01- 3.91 (m, 2H), 3.75- 3.57 (m, 1H), 2.29 (m, 1H), 2.16-2.01 (m, 5H), 1.80-1.68 (m, 2H), 1.67-1.58 (m, 2H), 1.56-1.49 (m, 1H), 1.35-1.28 (m, 3H), 1.02 (d, J = 6.0 Hz, 3H), 0.95- 0.89 (m, 3H) |
| 293 | propyl ((2,6- dimethylphenoxy) ((2-(((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl) phosphoryl)- L-alaninate | 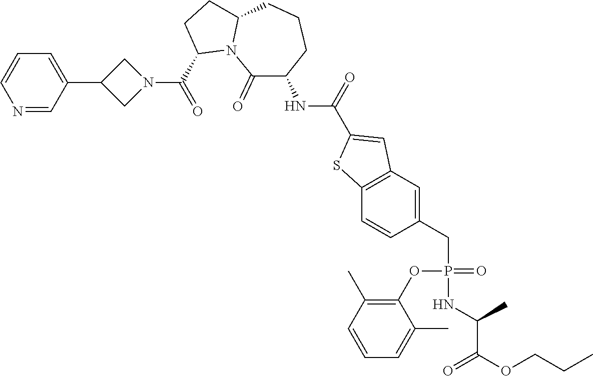 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1c(C)cccc1C] | 800.3 | ¹H NMR (400 MHz, CD₃OD) δ 8.60- 8.52 (m, 1H), 8.49- 8.38 (m, 1H), 8.12- 8.03 (m, 1.6H), 7.99- 7.92 (m, 1.4H), 7.91-7.85 (m, 1H), 7.55-7.45 (m, 1.4H), 7.44-7.38 (m, 0.6H), 7.03- 6.90 (m, 3H), 4.94 (t, J = 8.4 Hz, 0.4H), 4.80-4.73 (m, 1H), 4.72-4.66 (m, 0.6H), 4.63-4.47 (m, 2H), 4.45-4.37 (m, 0.6H), 4.34- 4.26 (m, 0.4H), 4.13- 3.76 (m, 6H), 3.69- 3.48 (m, 2H), 2.36- 2.25 (m, 5H), 2.22 (s, 2H), 2.20-1.78 (m, 9H), 1.59-1.46 (m, 2H), 1.15 (d, J = 7.3 Hz, 1H), 1.03 (d, J = 7.1 Hz, 2H), 0.91- 0.80 (m, 3H) |
| 294 | propyl 2-methyl-3- ((((2-(((3S,6S,9aS)- 5-oxo-3-(3- (pyridin-3- yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino) propanoate | 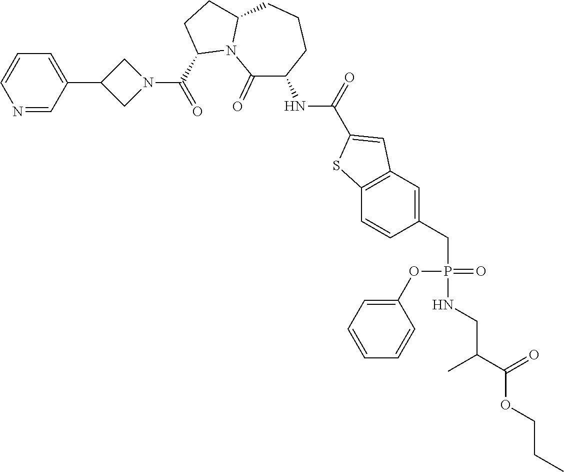 [SMILES: CCCOC(=O)C(C)CNP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 786.1 | ¹H NMR (400 MHz, CD₃OD) δ 8.60-8.51 (m, 1H), 8.49-8.39 (m, 1H), 8.14-7.83 (m, 4H), 7.53-7.38 (m, 2H), 7.38-7.27 (m, 2H), 7.23-7.08 (m, 3H), 4.98-4.91 (m, 0.4H), 4.79- 4.66 (m, 1.6H), 4.62- 4.48 (m, 2H), 4.46- 4.38 (m, 0.6H), 4.34- 4.27 (m, 0.4H), 4.13-3.84 (m, 5H), 3.57-3.42 (m, 2H), 3.15-3.00 (m, 1H), 2.98-2.80 (m, 1H), 2.39-2.26 (m, 2H), 2.20-1.78 (m, 9H), 1.61-1.50 (m, 2H), 1.01-0.92 (m, 3H), 0.92-0.83 (m, 3H) |
| 295 | butyl (2S)-4,4- difluoro-1-(((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl) pyrrolidine- 2-carboxylate | 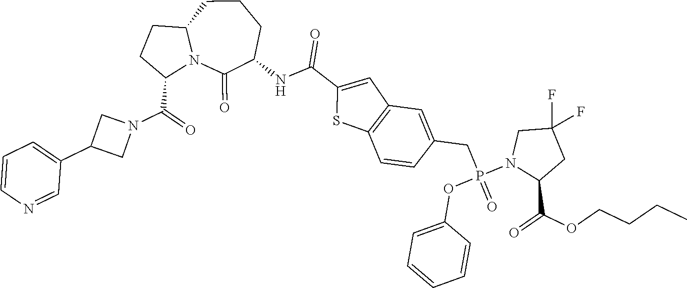 | 848.3 | ¹H NMR (400 MHz, CD₃OD) δ 8.63- 8.35 (m, 2H), 8.15- 7.85 (m, 4H), 7.62- 7.08 (m, 7H), 4.99- 4.90 (m , 0.4H), 4.80- 4.38 (m, 4.6H), 4.34-4.24 (m, 1H), 4.19-3.34 (m, 9H), 2.64-2.25 (m, 3H), 2.19-1.77 (m, 9H), 1.66-1.46 (m, 2H), 1.44-1.26 (m, 2H), 1.00-0.83 (m, 3H). |
| 296 | propyl (2S)-2-((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino) butanoate | 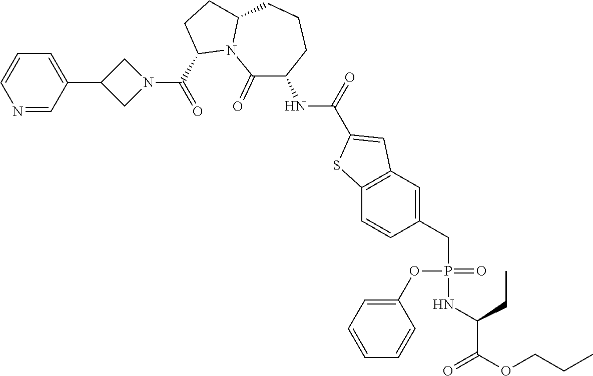 [SMILES: CCCOC(=O)[C@H](CC)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 786.2 | ¹H NMR (400 MHz, CD₃OD) δ 8.60- 8.52 (m, 1H), 8.50- 8.39 (m, 1H), 8.11- 7.85 (m, 4H), 7.54- 7.40 (m, 2H), 7.35- 7.27 (m, 2H), 7.22- 7.02 (m, 3H), 4.98- 4.91 (m, 0.4H), 4.79- 4.66 (m, 1.6H), 4.62-4.47 (m, 2H), 4.46-4.38 (m, 0.6H), 4.33-4.27 (m, 0.4H), 4.12- 3.97 (m, 3H), 3.96- 3.80 (m, 2H), 3.79- 3.68 (m, 1H), 3.62- 3.44 (m, 2H), 2.37- 2.26 (m, 1H), 2.19- 1.77 (m, 9H), 1.61- 1.39 (m, 4H), 0.90- 0.81 (m, 3H), 0.72- 0.63 (m, 3H) |
| 297 | benzyl ((difluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 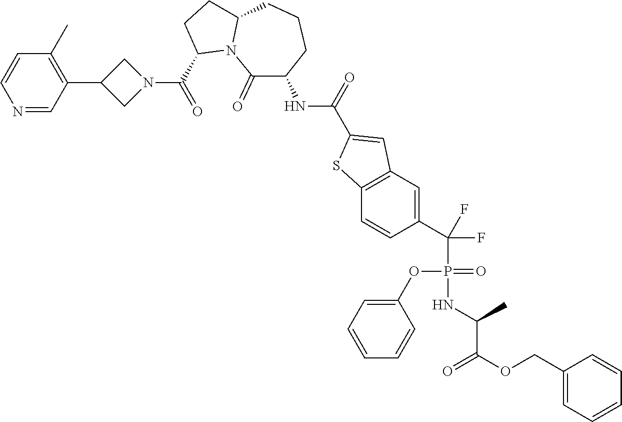 [SMILES: Cc1ccncc1C1CN(C(=O)[C@@H]2CC[C@@H]3CCC[C@H](NC(=O)c4cc5cc(C(F)(F)P(=O)(N[C@@H](C)C(=O)OCc6ccccc6)Oc6ccccc6)ccc5s4)C(=O)N32)C1] | 870.0 | ¹H NMR (400 MHz, CD₃OD) δ 8.64- 8.52 (m, 1H), 8.34- 8.25 (m, 1H), 8.23- 8.17 (m, 1H), 8.17- 8.10 (m, 1H), 8.09- 8.02 (m, 0.5H), 8.01- 7.95 (m, 0.5H), 7.75-7.66 (m, 1H), 7.40-7.10 (m, 11H), 5.10-5.06 (m, 1H), 5.06-4.98 (m, 1H), 4.98-4.91 (m, 0.5H), 4.79-4.68 (m, 2H), 4.64-4.56 (m, 1H), 4.54-4.47 (m, 0.5H), 4.45- 4.36 (m, 1H), 4.28- 4.02 (m, 3H), 4.01- 3.90 (m, 1H), 2.36- 2.25 (m, 4H), 2.21- 1.75 (m, 9H), 1.31- 1.24 (m, 3H) |
| 298 | isopropyl (((2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 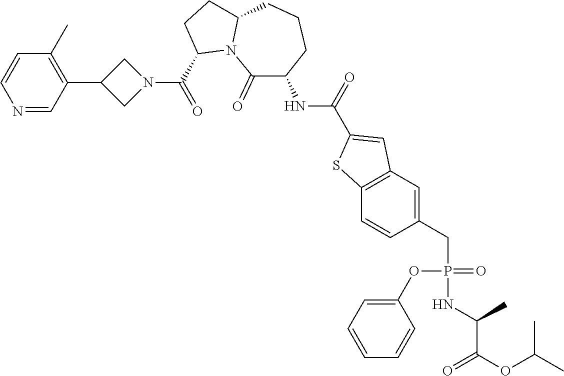 [SMILES: Cc1ccncc1C1CN(C(=O)[C@@H]2CC[C@@H]3CCC[C@H](NC(=O)c4cc5cc(CP(=O)(N[C@@H](C)C(=O)OC(C)C)Oc6ccccc6)ccc5s4)C(=O)N32)C1] | 786.3 | ¹H NMR (400 MHz, DMSO-d₆) δ 8.76 (t, J = 8.6 Hz, 1H), 8.59- 8.50 (m, 1H), 8.36- 8.29 (m, 1H), 8.22- 8.16 (m, 1H), 7.93 (q, J = 7.1 Hz, 1H), 7.90-7.86 (m, 1H), 7.47-7.41 (m, 1H), 7.36-7.29 (m, 2H), 7.22-7.08 (m, 4H), 5.70-5.50 (m, 1H), 4.85-4.75 (m, 1.5H), 4.70-4.59 (m, 1.5H), 4.50- 4.42 (m, 1.5H), 4.38- 4.21 (m, 1.5H), 4.17-3.96 (m, 3H), 3.86-3.65 (m, 1H), 3.50-3.38 (m, 2H), 2.20 (s, 4H), 2.09- 1.92 (m, 2H), 1.88- 1.63 (m, 7H), 1.13- 1.05 (m, 7H), 0.97 (t, J = 6.6 Hz, 2H) |
| 299 | propyl (((2- (((3S,6S,10aS)-3- (rel-(trans)-3- cyano-4-(1-methyl- 2-oxo-1,2- dihydropyridin-3- yl)pyrrolidine-1- carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 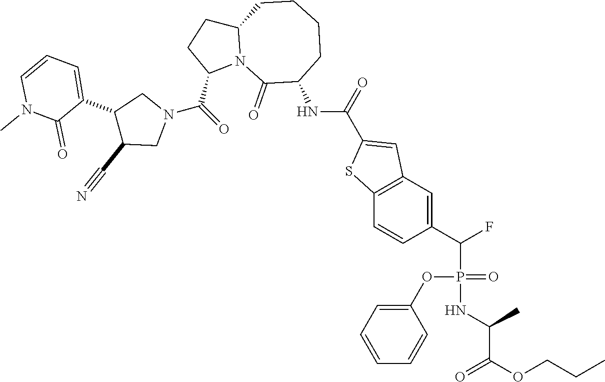 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@@H](C#N)[C@H](c6cccn(C)c6=O)C5)N4C3=O)cc2c1] | 873.1 | ¹H NMR (400 MHz, CD₃OD) δ 8.18- 8.09 (m, 1H), 8.09- 8.04 (m, 1H), 8.03- 7.90 (m, 1H), 7.68- 7.49 (m, 3H), 7.38- 7.27 (m, 2H), 7.24- 7.11 (m, 3H), 6.42- 6.28 (m, 1H), 6.20- 5.80 (m, 1H), 5.10- 4.98 (m, 1H), 4.72- 4.57 (m, 1H), 4.49- 4.36 (m, 1H), 4.29- 3.61 (m, 8H), 3.59- 3.49 (m, 3H), 2.42- 2.17 (m, 2H), 2.10- 1.47 (m, 11H), 1.40- 1.01 (m, 5H), 0.90- 0.81 (m, 3H) |
| 300 | propyl (((2- (((3S,6S,9aS)-3-(3- (4-(3- methoxyazetidin-1- yl)pyridin-3- yl)azetidine-1- carbonyl)-5- oxooctahydro-1H- pyrrolo[1,2- aJazepin-6- yl)carbamoyl)benzo [b]thiophen-5- yl)methyl)(phenoxy) phosphoryl)-L- alaninate | 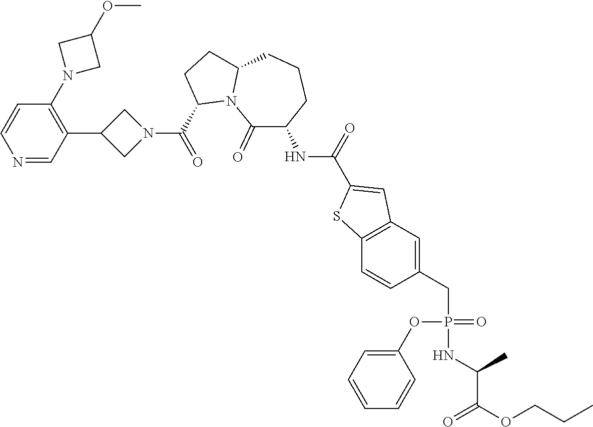 [SMILES: CCCOC(=O)[C@H](C)NP(=O)(Cc1ccc2sc(C(=O)N[C@H]3CCC[C@H]4CC[C@@H](C(=O)N5CC(c6cnccc6N6CC(OC)C6)C5)N4C3=O)cc2c1)Oc1ccccc1] | 857.2 | ¹H NMR (400 MHz, CD₃OD) δ 8.40- 8.23 (m, 1H), 8.13- 7.99 (m, 2H), 7.95- 7.82 (m, 2H), 7.54- 7.45 (m, 1H), 7.38- 7.26 (m, 2H), 7.24- 7.09 (m, 3H), 6.55- 6.35 (m, 1H), 4.78- 4.74 (m, 1H), 4.66- 4.54 (m, 2H), 4.50- 4.35 (m, 1H), 4.35- 4.25 (m, 4H), 4.16- 4.01 (m, 3H), 4.01- 3.76 (m, 5H), 3.60- 3.42 (m, 2H), 3.36- 3.29 (m, 3H), 2.37- 2.25 (m, 1H), 2.19- 1.73 (m, 9H), 1.59- 1.48 (m, 2H), 1.18- 1.05 (m, 3H), 0.90- 0.83 (m, 3H) |
| 301 | propyl (((2- (((3S,6S,9aS)-3-(3- (4-((S)-2- (methoxymethyl) azetidin-1-yl)pyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 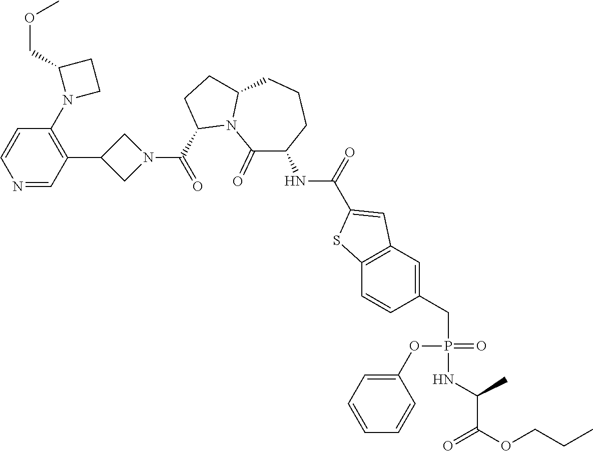 [SMILES: CCCOC(=O)[C@H](C)NP(=O)(Cc1ccc2sc(C(=O)N[C@H]3CCC[C@H]4CC[C@@H](C(=O)N5CC(c6cnccc6N6CC[C@H]6COC)C5)N4C3=O)cc2c1)Oc1ccccc1] | 871.2 | ¹H NMR (400 MHz, CD₃OD) δ 8.37- 8.24 (m, 1H), 8.12- 8.00 (m, 2H), 7.96- 7.83 (m, 2H), 7.53- 7.44 (m, 1H), 7.36- 7.27 (m, 2H), 7.21 (m, 3H), 6.81-6.72 (m, 1H), 4.78-4.54 (m, 4H), 4.52-4.43 (m, 1H), 4.38-3.99 (m, 5H), 3.98-3.77 (m, 3H), 3.75-3.64 (m, 1H), 3.64-3.41 (m, 4H), 3.37-3.32 (m, 3H), 2.46-1.74 (m, 12H), 1.62-1.47 (m, 2H), 1.18-1.05 (m, 3H), 0.90-0.82 (m, 3H) |
| 302 | propyl (((2- (((3′S,6′S,10a′S)-5′- oxo-3′-(3-(pyridin- 3-yl)azetidine-1- carbonyl)octahydro- 5′H- spiro[cyclopropane- 1,8′-pyrrolo[1,2- a]azocin]-6′- yl)carbamoyl)benzo [b]thiophen-5- yl)methyl)(phenoxy) phosphoryl)-L- alaninate | 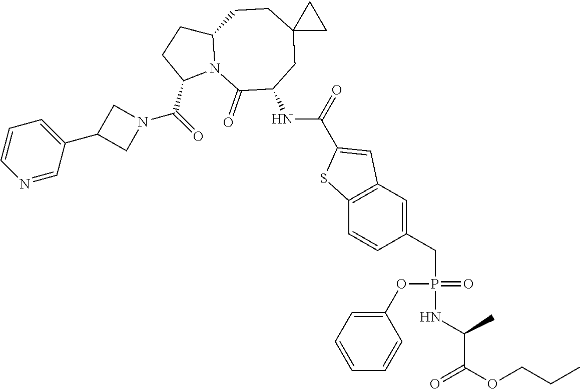 [SMILES: CCCOC(=O)[C@H](C)NP(=O)(Cc1ccc2sc(C(=O)N[C@H]3CC4(CC[C@H]5CC[C@@H](C(=O)N6CC(c7cccnc7)C6)N5C3=O)CC4)cc2c1)Oc1ccccc1] | 812.2 | ¹H NMR (400 MHz, CD₃OD) δ 8.60 (br. s., 0.5H), 8.54 (s, 0.5H), 8.49-8.40 (m, 1H), 8.08-8.01 (m, 1.5H), 7.97- 7.82 (m, 2.5H), 7.53- 7.41 (m, 2H), 7.36- 7.27 (m, 2H), 7.22- 7.11 (m, 3H), 5.07- 4.96 (m, 1.5H), 4.70 (d, J = 7.3 Hz, 1H), 4.58-4.37 (m, 3H), 4.34-4.26 (m, 0.5H), 4.10-3.76 (m, 5H), 3.60-3.45 (m, 2H), 2.33-2.16 (m, 2H), 2.15-2.01 (m, 1H), 2.01-1.82 (m, 4H), 1.77-1.46 (m, 5H), 1.14 (d, J = 7.1 Hz, 1H), 1.10- 1.04 (m, 2H), 0.91- 0.81 (m, 3H), 0.64- 0.43 (m, 4H) |
| 303 | propyl (((R)- fluoro(2- (((3S,6S,10aS)-3- (6-(5- methylpyridin-3- yl)-5,7-dioxo-4,6- diazaspiro [2.4]heptane- 4-carbonyl)-5- oxodecahydropyrrolo [1,2-a]azocin-6- yl)carbamoyl)benzo [b]thiophen-5- yl)methyl)(phenoxy) phosphoryl)-L- alaninate | 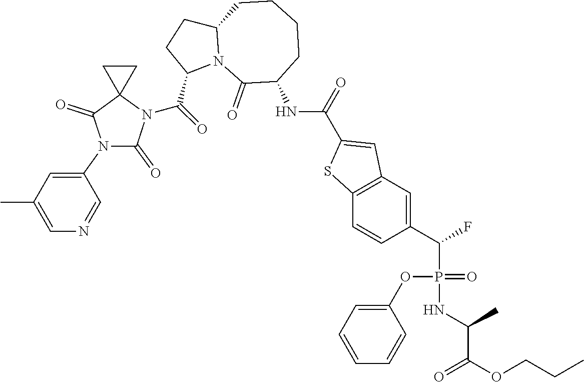 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6cncc(C)c6)C(=O)C56CC6)N4C3=O)cc2c1] | 887.1 | ¹H NMR (400 MHz, CD₃OD) δ 8.75- 8.37 (m, 1H), 8.14- 8.05 (m, 2H), 8.01- 7.95 (m, 1H), 7.87- 7.80 (m, 1H), 7.67- 7.59 (m, 1H), 7.37- 7.29 (m, 2H), 7.23- 7.12 (m, 3H), 6.18- 5.99 (m, 1H), 5.61- 5.53 (m, 1H), 5.09- 5.00 (m, 1H), 4.53- 4.43 (m, 1H), 4.00- 3.67 (m, 3H), 3.26- 3.11 (m, 1H), 2.63- 2.52 (m, 1H), 2.46- 2.39 (m, 4H), 2.34- 2.28 (m, 1H), 2.07- 1.81 (m, 5H), 1.76- 1.62 (m, 1H), 1.62- 1.45 (m, 2H), 1.40- 1.34 (m, 3H), 1.33- 1.25 (m, 3H), 1.23- 1.18 (m, 2H), 0.98- 0.82 (m, 4H) |
| 304 | propyl (((2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 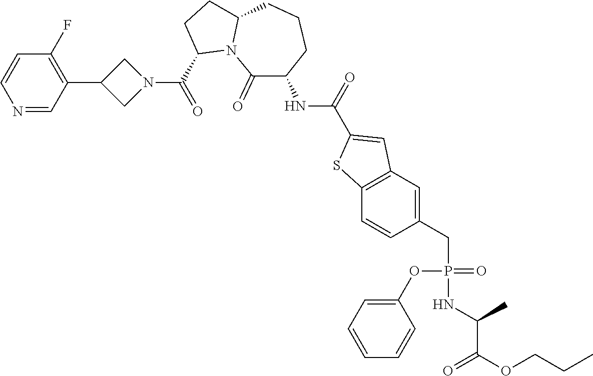 [SMILES: CCCOC(=O)[C@H](C)NP(=O)(Cc1ccc2sc(C(=O)N[C@H]3CCC[C@H]4CC[C@@H](C(=O)N5CC(c6cnccc6F)C5)N4C3=O)cc2c1)Oc1ccccc1] | 790.3 | ¹H NMR (400 MHz, CD₃OD) δ 8.77- 8.57 (m, 1H), 8.54- 8.43 (m, 1H), 8.04 (d, J = 3.9 Hz, 1H), 7.95-7.82 (m, 2H), 7.53-7.43 (m, 1H), 7.36-7.09 (m, 6H), 4.92 (t, J = 8.7 Hz, 0.5H), 4.78-4.67 (m, 2H), 4.61-4.54 (m, 1H), 4.53-4.36 (m, 1.5H), 4.26- 4.11 (m, 2H), 4.10- 4.01 (m, 1H), 3.98- 3.76 (m, 3H), 3.60- 3.41 (m, 2H), 2.37- 2.24 (m, 1H), 2.20- 1.74 (m, 9H), 1.60- 1.47 (m, 2H), 1.14 (d, J = 7.1 Hz, 1.25H), 1.07 (dd, J = 7.1, 2.2 Hz, 1.75H), 0.91-0.81 (m, 3H) |
| 305 | propyl (((7- (((3S,6S,10aS)-5- oxo-3-(3-(pyridin- 3-yl)pyrrolidine-1- carbonyl)decahydro pyrrolo[1,2- a]azocin-6- yl)carbamoyl) naphthalen-2- yl)methyl)(phenoxy) phosphoryl)-L- alaninate | 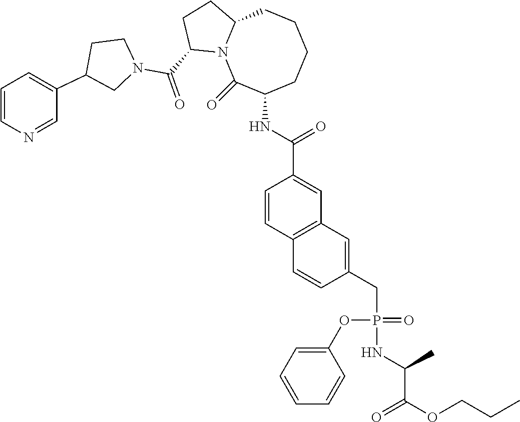 [SMILES: CCCOC(=O)[C@H](C)NP(=O)(Cc1ccc2ccc(C(=O)N[C@H]3CCCC[C@H]4CC[C@@H](C(=O)N5CCC(c6cccnc6)C5)N4C3=O)cc2c1)Oc1ccccc1] | 794.2 | ¹H NMR (400 MHz, CD₃OD) δ 8.56- 8.35 (m, 3H), 8.01- 7.75 (m, 5H), 7.67- 761 (m, 1H), 7.45- 7.27 (m, 3H), 7.24- 7.10 (m, 3H), 5.18- 5.07 (m, 1H), 4.78- 4.65 (m, 1H), 4.53- 4.35 (m, 1H), 4.16- 3.76 (m, 5H), 3.75- 3.33 (m, 5H), 2.47- 1.93 (m, 10H), 1.92- 1.75 (m, 2H), 1.74- 1.58 (m, 2H), 1.58- 1.43 (m, 2H), 1.19- 1.00 (m, 3H), 0.92- 0.75 (m, 3H) |
| 306 | propyl ((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 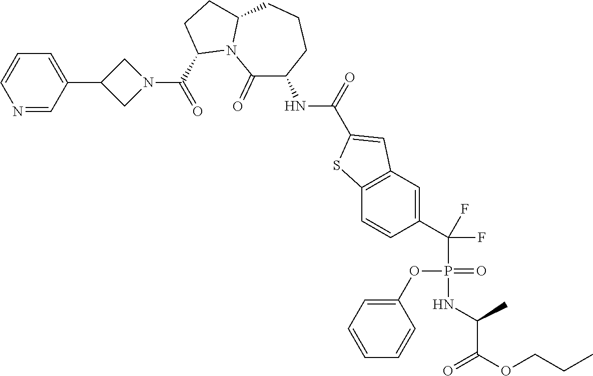 [SMILES: CCCOC(=O)[C@H](C)NP(=O)(Oc1ccccc1)C(F)(F)c1ccc2sc(C(=O)N[C@H]3CCC[C@H]4CC[C@@H](C(=O)N5CC(c6cccnc6)C5)N4C3=O)cc2c1] | 809.1 | ¹H NMR (400 MHz, CD₃OD) δ 8.60- 8.52 (m, 1H), 8.49- 8.38 (m, 1H), 8.29- 7.93 (m, 4H), 7.78- 7.67 (m, 1H), 7.51- 7.31 (m, 3H), 7.27- 7.02 (m, 3H), 5.09- 4.91 (m, 1.4H), 4.80- 4.74 (m, 1H), 4.73- 4.66 (m, 0.6H), 4.63- 4.47 (m, 2H), 4.46- 4.27 (m, 1H), 4.15- 3.95 (m, 5H), 2.37- 2.26 (m, 1H), 2.20- 1.79 (m, 9H), 1.63- 1.53 (m, 2H), 1.52- 1.44 (m, 3H), 0.92- 0.83 (m, 3H) |
| 307 | propyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 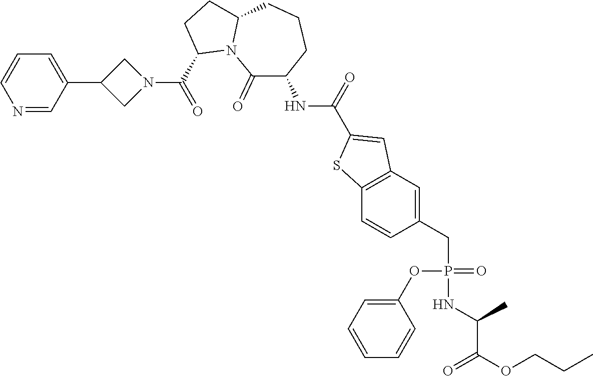 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 773.1 | ¹H NMR (400 MHz, CD₃OD) δ 8.59- 8.55 (m, 1H), 8.50- 8.37 (m, 1H), 8.11- 7.84 (m, 4H), 7.56- 7.38 (m, 2H), 7.37- 7.26 (m, 2H), 7.24- 7.11 (m, 2H), 7.10- 7.03 (m, 1H), 4.98- 4.86 (m, 1.4H), 4.80- 4.65 (m, 1.6H), 4.62-4.37 (m, 2.6H), 4.30 (dd, J = 9.4, 5.3 Hz, 0.4H), 4.16-3.95 (m, 5H), 3.80-3.58 (m, 2H), 2.37-2.26 (m, 1H), 2.19-1.77 (m, 9H), 1.69-1.51 (m, 2H), 1.36 (dd, J = 6.8, 1.5 Hz, 3H), 0.98-0.80 (m, 3H) |
| 308 | propyl (((2- (((3S,6S,9aS)-3- (((3S,4S)-1-acetyl- 4-fluoropyrrolidin- 3-yl)carbamoyl)-5- oxooctahydro-1H- pyrrolo[1,2- a]azepin-6- yl)carbamoyl)benzo [b]thiophen-5- yl)methyl)(phenoxy) phosphoryl)-L- alaninate | 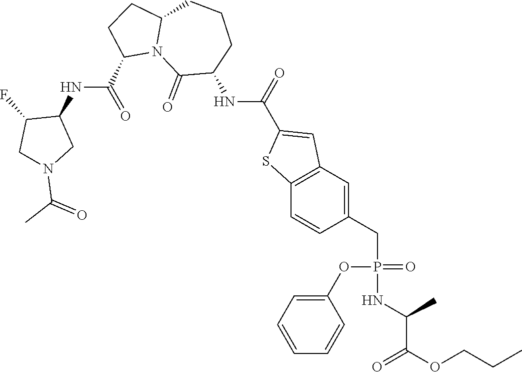 [SMILES: CCCOC(=O)[C@H](C)NP(=O)(Cc1ccc2sc(C(=O)N[C@H]3CCC[C@H]4CC[C@@H](C(=O)N[C@H]5CN(C(C)=O)C[C@@H]5F)N4C3=O)cc2c1)Oc1ccccc1] | 784.3 | ¹H NMR (400 MHz, CD₃OD) δ 8.04- 8.00 (m, 1H), 7.93- 7.84 (m, 2H), 7.52- 7.44 (m, 1H), 7.36- 7.27 (m, 2H), 7.21- 7.11 (m, 3H), 5.19- 4.94 (m, 1H), 4.73 (d, J = 10.8 Hz, 1H), 4.54-4.36 (m, 2H), 4.11-3.99 (m, 1H), 3.98-3.42 (m, 9H), 2.34-2.22 (m, 1H), 2.17-1.73 (m, 12H), 1.61-1.46 (m, 2H), 1.14 (d, J = 7.1 Hz, 1.15H), 1.07 (d, J = 7.1 Hz, 1.85H), 0.86 (t, J = 7.5 Hz, 3H) |
| 309 | propyl (((2- (((3S,6S,9aS)-3- (((3S,4R)-1-acetyl- 4-fluoropyrrolidin- 3-yl)carbamoyl)-5- oxooctahydro-1H- pyrrolo[1,2- a]azepin-6- yl)carbamoyl)benzo [b]thiophen-5- yl)methyl)(phenoxy) phosphoryl)-L- alaninate | 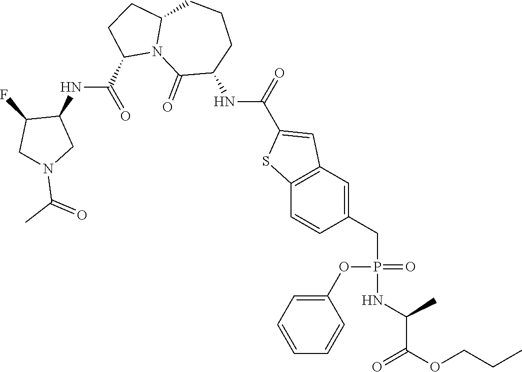 [SMILES: CCCOC(=O)[C@H](C)NP(=O)(Cc1ccc2sc(C(=O)N[C@H]3CCC[C@H]4CC[C@@H](C(=O)N[C@H]5CN(C(C)=O)C[C@H]5F)N4C3=O)cc2c1)Oc1ccccc1] | 784.3 | ¹H NMR (400 MHz, CD₃OD) δ 8.05- 8.00 (m, 1H), 7.94- 7.84 (m, 2H), 7.53- 7.45 (m, 1H), 7.36- 7.27 (m, 2H), 7.21- 7.11 (m, 3H), 5.29- 5.04 (m, 1H), 4.74 (d, J = 10.5 Hz, 1H), 4.67-4.42 (m, 2H), 4.11-4.02 (m, 1H), 4.01-3.64 (m, 6H), 3.61-3.37 (m, 3H), 2.36-2.24 (m, 1H), 2.19-1.72 (m, 12H), 1.59-1.47 (m, 2H), 1.14 (d, J = 7.1 Hz, 1.25H), 1.07 (d, J = 7.1 Hz, 1.75H), 0.86 (t, J = 7.3 Hz, 3H) |
| 310 | dipropyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) (S)-di-L- prolinate | 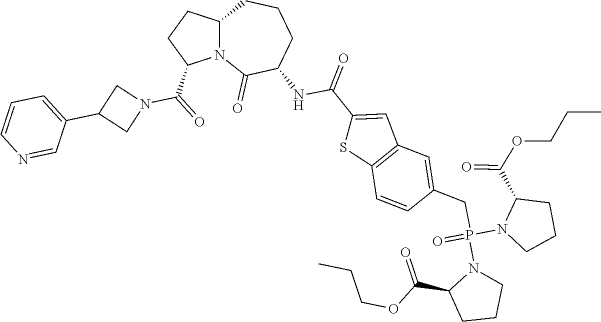 [SMILES: CCCOC(=O)[C@@H]1CCCN1P(=O)(Cc1ccc2sc(C(=O)N[C@H]3CCC[C@H]4CC[C@@H](C(=O)N5CC(c6cccnc6)C5)N4C3=O)cc2c1)N1CCC[C@H]1C(=O)OCCC] | 861.2 | ¹H NMR (400 MHz, CD₃OD) δ 8.59- 8.53 (m, 1H), 8.49- 8.41 (m, 1H), 8.13- 8.08 (m, 0.5H), 8.07- 8.03 (m, 1H), 7.99- 7.94 (m, 0.5H), 7.93- 7.86 (m, 2H), 7.51- 7.40 (m, 2H), 4.98- 4.93 (m, 0.5H), 4.79- 4.67 (m, 1H), 4.63- 4.55 (m, 1.5H), 4.46- 4.37 (m, 1.5H), 4.35-4.26 (m, 0.5H), 4.18-3.90 (m, 7H), 3.71-3.64 (m, 1H), 3.62-3.47 (m, 2H), 3.46-3.35 (m, 3H), 2.36-1.75 (m, 19H), 1.74-1.65 (m, 2H), 1.63-1.56 (m, 2H), 1.01-0.94 (m, 3H), 0.92-0.86 (m, 3H) |
| 311 | propyl 3-((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino) butanoate | 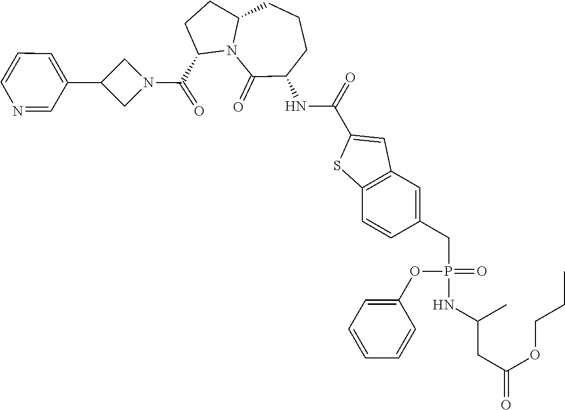 [SMILES: CCCOC(=O)CC(C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 786.1 | ¹H NMR (400 MHz, CD₃OD) δ 8.62- 8.55 (m, 1H), 8.52- 5.40 (m, 1H), 8.16- 7.84 (m, 4H), 7.54- 7.41 (m, 2H), 7.38- 7.26 (m, 2H), 7.22- 7.08 (m, 3H), 4.99- 4.91 (m, 0.4H), 4.80- 4.66 (m, 1.6H), 4.63-4.48 (m, 2H), 4.46-4.36 (m, 0.6H), 4.33-4.27 (m, 0.4H), 4.13- 3.84 (m, 5H), 3.74- 3.38 (m, 3H), 2.39- 1.74 (m, 12H), 1.62- 1.49 (m, 2H), 1.00- 0.92 (m, 3H), 0.91- 0.83 (m, 3H) |
| 312 | propyl ((difluoro(7- (((3S,6S,10aS)-5- oxo-3-(3-(pyridin- 3-yl)pyrrolidine-1- carbonyl)decahydro pyrrolo[1,2- a]azocin-6- yl)carbamoyl) naphthalen-2- yl)methyl)(phenoxy) phosphoryl)-L- alaninate | 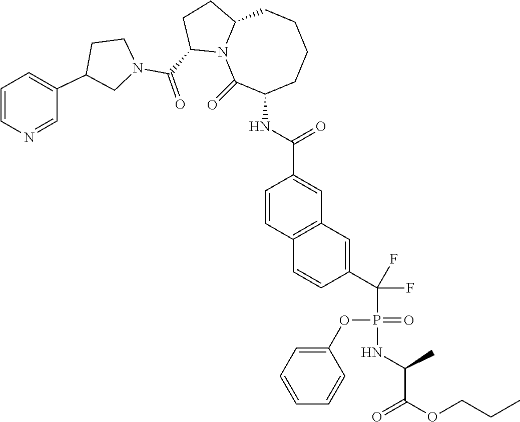 [SMILES: CCCOC(=O)[C@H](C)NP(=O)(Oc1ccccc1)C(F)(F)c1ccc2ccc(C(=O)N[C@H]3CCCC[C@H]4CC[C@@H](C(=O)N5CCC(c6cccnc6)C5)N4C3=O)cc2c1] | 830.3 | ¹H NMR (400 MHz, CD₃OD) δ 8.62- 8.46 (m, 2H), 8.46- 8.35 (m, 1H), 8.34- 8.28 (m , 1H), 8.13- 7.97 (m, 3H), 7.91- 7.76 (m, 2H), 7.47- 7.27 (m, 3H), 7.24- 7.08 (m, 3H), 5.18- 5.07 (m, 1H), 4.79- 4.67 (m, 1H), 4.53- 4.36 (m, 1H), 4.17- 3.77 (m, 5H), 3.75- 3.33 (m, 3H), 2.49- 1.94 (m, 10H), 1.93- 1.76 (m, 2H), 1.75- 1.46 (m, 4H), 1.32- 1.23 (m, 3H), 0.94- 0.79 (m, 3H) |
| 313 | propyl ((1-fluoro-1- (2-(((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)ethyl)(phenoxy) phosphoryl)-L- alaninate | 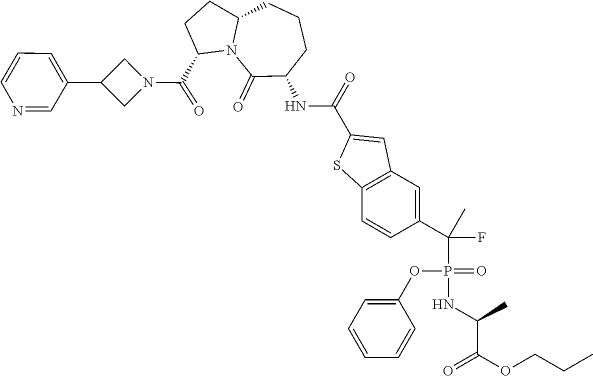 [SMILES: CCCOC(=O)[C@H](C)NP(=O)(Oc1ccccc1)C(C)(F)c1ccc2sc(C(=O)N[C@H]3CCC[C@H]4CC[C@@H](C(=O)N5CC(c6cccnc6)C5)N4C3=O)cc2c1] | 804.3 | ¹H NMR (400 MHz, CD₃OD) δ 8.60- 8.52 (m, 1H), 8.50- 8.39 (m, 1H), 8.16- 8.05 (m, 2.5H), 8.03- 7.93 (m, 1.5H), 7.72-7.64 (m, 1H), 7.52-7.32 (m, 2H), 7.31-7.16 (m, 2.5H), 7.15-7.00 (m, 1.5H), 4.99- 4.91 (m, 0.5H), 4.81- 4.73 (m, 1H), 4.73- 4.66 (m, 0.5H), 4.64- 4.50 (m, 2H), 4.46- 4.37 (m, 0.5H), 4.35- 4.26 (m, 0.5H), 4.14-3.85 (m, 5H), 2.38-2.25 (m, 1H), 2.18-1.77 (m, 12H), 1.64-1.53 (m, 1H), 1.52-1.38 (m, 1H), 1.33-1.26 (m, 1H), 1.24 (d, J = 7.3 Hz, 1H), 1.20 (d, J = 7.1 Hz, 1H), 1.10 (d, J = 7.1 Hz, 1H), 0.94- 0.87 (m, 2H), 0.85- 0.77 (m, 1H) |
| 314 | propyl ((1-fluoro-1- (2-(((3S,6S,10aS)- 5-oxo-3-(3- (pyridin-3- yl)pyrrolidine-1- carbonyl)decahydro pyrrolo[1,2- a]azocin-6- yl)carbamoyl)benzo [b]thiophen-5- yl)ethyl)(phenoxy) phosphoryl)-L- alaninate | 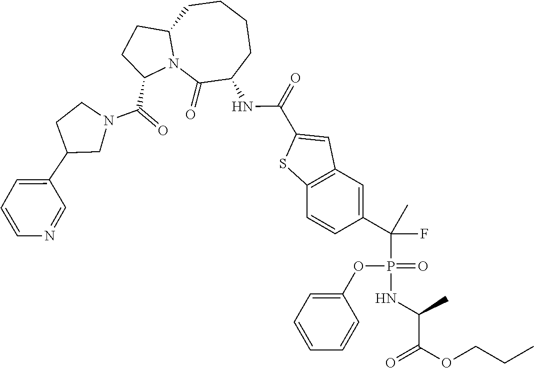 [SMILES: CCCOC(=O)[C@H](C)NP(=O)(Oc1ccccc1)C(C)(F)c1ccc2sc(C(=O)N[C@H]3CCCC[C@H]4CC[C@@H](C(=O)N5CCC(c6cccnc6)C5)N4C3=O)cc2c1] | 832.3 | ¹H NMR (400 MHz, CD₃OD) δ 8.57- 8.47 (m, 1H), 8.46- 8.35 (m, 1H), 8.19- 8.04 (m, 2H), 8.02- 7.91 (m, 1H), 7.90- 7.78 (m, 1H), 7.71- 7.61 (m, 1H), 7.46- 7.32 (m, 2H), 7.31- 7.16 (m, 2.4H), 7.16- 6.98 (m, 1.6H), 5.12-5.00 (m, 1H), 4.79-4.64 (m, 1H), 4.50-4.35 (m, 1H), 4.16-3.76 (m, 4H), 3.75-3.41 (m, 3H), 2.50-2.18 (m, 3H), 2.17-1.93 (m, 10H), 1.92-1.52 (m, 6H), 1.51-1.36 (m, 1H), 1.27-1.17 (m, 2H), 1.13-1.07 (m, 1H), 0.95-0.86 (m, 2H), 0.85-0.77 (m, 1H) |
| 315 | propyl (((2- (((3S,6S,10aS)-3- ((3S,4R)-3-cyano- 4- phenylpyrrolidine- 1-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((2- (((3S,6S,10aS)-3- ((3R,4S)-3-cyano-4- phenylpyrrolidine- 1-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 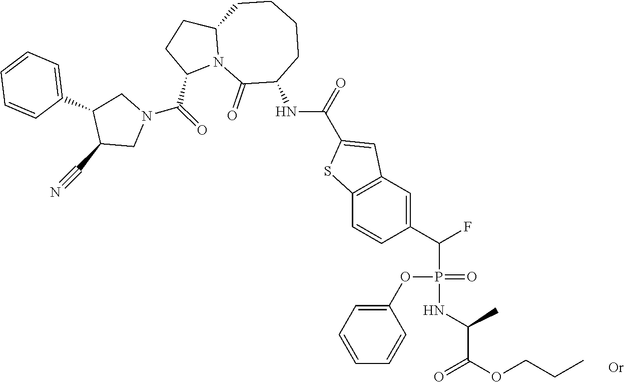 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@@H](C#N)[C@H](c6ccccc6)C5)N4C3=O)cc2c1] 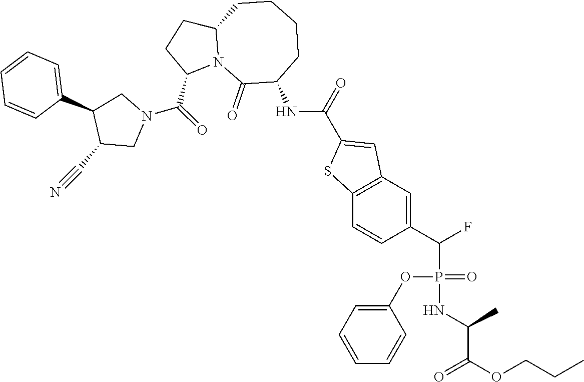 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@H](c6ccccc6)[C@@H](C#N)C5)N4C3=O)cc2c1] Pyrrolidine building block SFC peak 2 was used for synthesis and biological testing | 842.1 | ¹H NMR (400 MHz, CD₃OD) δ 8.14 (s, 1H), 8.08 (br. s., 1H), 8.04-7.92 (m, 1H), 7.70-7.59 (m, 1H), 7.46-7.27 (m, 7H), 7.25-7.13 (m, 3H), 6.20-5.98 (m, 1H), 5.09-4.99 (m, 1H), 4.73-4.65 (m, 1H), 4.57-4.50 (m, 0.5H), 4.49-4.39 (m, 1.5H), 4.10- 4.02 (m, 0.5H), 4.00- 3.64 (m, 5.5H), 3.63-3.49 (m, 2H), 2.42-2.15 (m, 2H), 2.10-1.92 (m, 6H), 1.91-1.76 (m, 2H), 1.73-1.48 (m, 4H), 1.25-1.18 (m, 2H), 1.09-1.02 (m, 1H), 0.93-0.80 (m, 3H) |
| 316 | propyl (((7- (((3S,6S,10aS)-3- ((3S,4R)-3-cyano- 4- phenylpyrrolidine- 1-carbonyl)-5- oxodecahydropyrrolo [1,2-a]azocin-6- yl)carbamoyl) naphthalen-2- yl)fluoromethyl) (phenoxy)phosphoryl)- L-alaninate Or propyl (((7- (((3S,6S,10aS)-3- ((3R,4S)-3-cyano-4- phenylpyrrolidine- 1-carbonyl)-5- oxodecahydropyrrolo [1,2-a]azocin-6- yl)carbamoyl) naphthalen-2- yl)fluoromethyl) (phenoxy)phosphoryl)- L-alaninate | 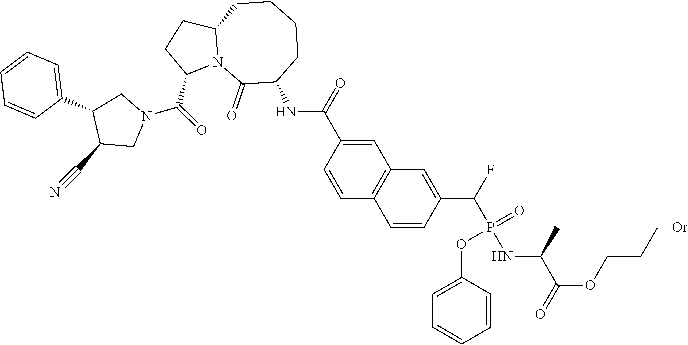 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5C[C@@H](C#N)[C@H](c6ccccc6)C5)N4C3=O)cc2c1] 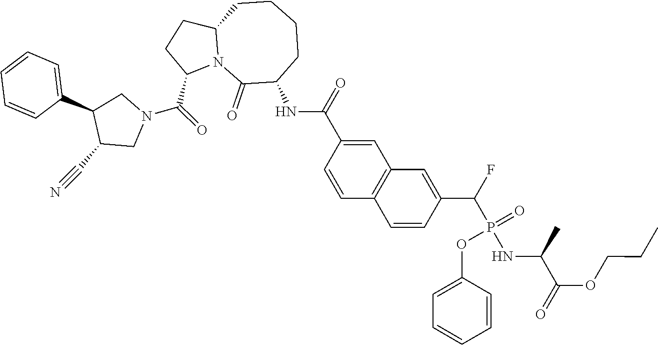 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5C[C@H](c6ccccc6)[C@@H](C#N)C5)N4C3=O)cc2c1] Pyrrolidine building block SFC peak 2 was used for synthesis and biological testing | 836.2 | ¹H NMR (400 MHz, CD₃OD) δ 8.53- 8.47 (m, 1H), 8.20- 8.10 (m, 1H), 8.05- 7.94 (m, 3H), 7.84- 7.73 (m, 1H), 7.68- 7.12 (m, 10H), 6.27- 6.06 (m, 1H), 5.16- 5.06 (m, 1H), 4.75- 4.64 (m, 1H), 4.60- 4.42 (m, 2H), 4.11- 3.47 (m, 8H), 2.42- 2.18 (m, 2H), 2.14- 1.92 (m, 6H), 1.92- 1.76 (m, 2H), 1.75- 1.63 (m, 2H), 1.60- 1.43 (m, 2H), 1.26- 1.15 (m, 2H), 1.09- 1.03 (m, 1H), 0.92- 0.78 (m, 3H) |
| 317 | propyl (((2- (((3S,6S,10aS)-3- ((3S,4R)-3-cyano- 4- phenylpyrrolidine- 1-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((2- (((3S,6S,10aS)-3- ((3R,4S)-3-cyano-4- phenylpyrrolidine- 1-carbonyl)-5- oxodecahydropyrrolo [1,2-a]azocin-6- yl)carbamoyl)benzo [b]thiophen-5- yl)fluoromethyl) | 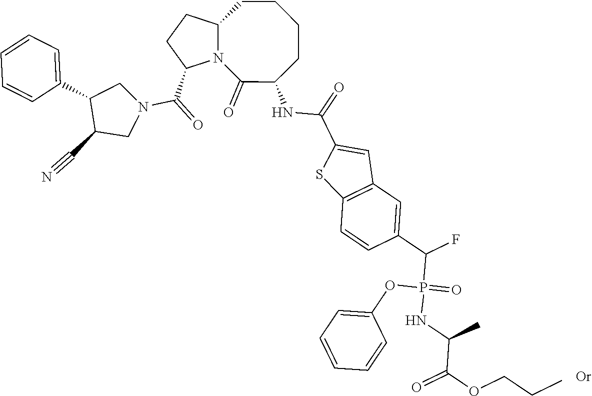 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 842.1 | ¹H NMR (400 MHz, CD₃OD) δ 8.17- 8.11 (m, 1H), 8.08 (br. s., 1H), 8.03- 7.92 (m, 1H), 7.69- 7.58 (m, 1H), 7.48- 7.26 (m, 7H), 7.24- 7.12 (m, 3H), 6.19- 5.98 (m, 1H), 5.09- 4.98 (m, 1H), 4.69- 4.57 (m, 1H), 4.49- 4.38 (m, 1H), 4.24- 4.15 (m, 1H), 4.14- 4.04 (m, 1.5H), 4.01- 3.73 (m, 4H), 3.70- 3.54 (m, 1.5H), 3.51- 3.37 (m, 1H), 2.40- 2.18 (m, 2H), 2.09- 1.94 (m, 6H), 1.90- 1.75 (m, 2H), 1.74- 1.44 (m, 4H), 1.27- 1.19 (m, 2H), 1.06 (d, J = 7.1 Hz, 1H), 0.92-0.81 (m, 3H) |
| | (phenoxy)phosphoryl)- L-alaninate | 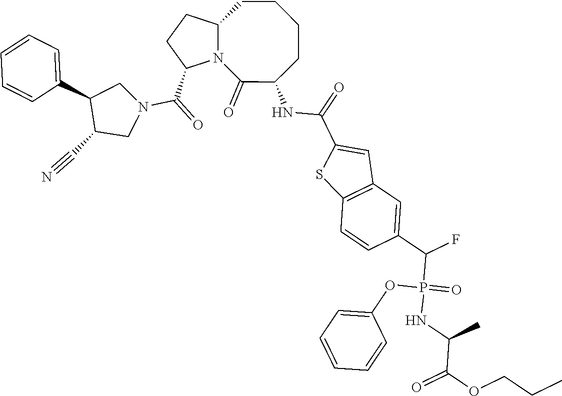 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@H](c6ccccc6)[C@@H](C#N)C5)N4C3=O)cc2c1] Pyrrolidine building block SFC peak 1 was used for synthesis and biological testing | | |
| 318 | propyl (((2- (((3S,6S,10aS)-3- ((3S,4R)-3-cyano- 4-(1-methyl-2-oxo- 1,2-dihydropyridin- 4-yl)pyrrolidine-1- carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((2- (((35,6S,10aS)-3- ((3R,4S)-3-cyano-4- (1-methyl-2-oxo- 1,2-dihydropyridin- 4-yl)pyrrolidine-1- carbonyl)-5- oxodecahydropyrrolo [1,2-a]azocin-6- | 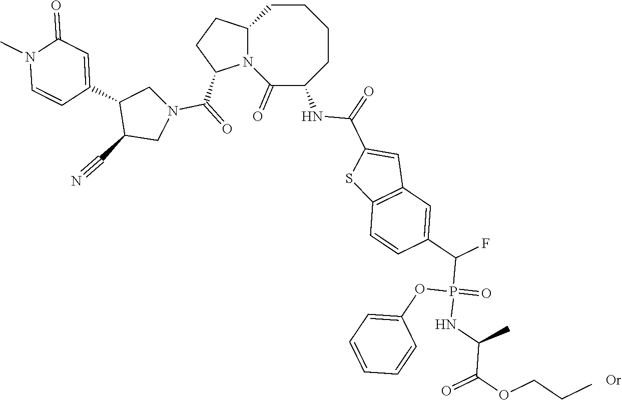 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@@H](C#N)[C@H](c6ccn(C)c(=O)c6)C5)N4C3=O)cc2c1] | 873.1 | ¹H NMR (400 MHz, CD₃OD) δ 8.16- 8.11 (m, 1H), 8.08 (br. s., 1H), 8.04- 7.93 (m, 1H), 7.71- 7.58 (m, 2H), 7.38- 7.28 (m, 2H), 7.25- 7.13 (m, 3H), 6.67- 6.55 (m, 1H), 6.50- 6.39 (m, 1H), 6.20- 5.98 (m, 1H), 5.09- 4.97 (m, 1H), 4.67- 4.56 (m, 1H), 4.50- 4.38 (m, 1H), 4.24- 4.14 (m, 1H), 4.13- 4.03 (m, 1.5H), 4.01- 3.77 (m, 3.5H), 3.76-3.65 (m, 1H), 3.64-3.44 (m, 4.5H), 3.41-3.33 (m, 0.5H), 2.41- 2.18 (m, 2H), 2.11- 1.91 (m, 6H), 1.91- 1.75 (m, 2H), 1.74- |
| | yl)carbamoyl)benzo [b]thiophen-5- yl)fluoromethyl) (phenoxy)phosphoryl)- L-alaninate | 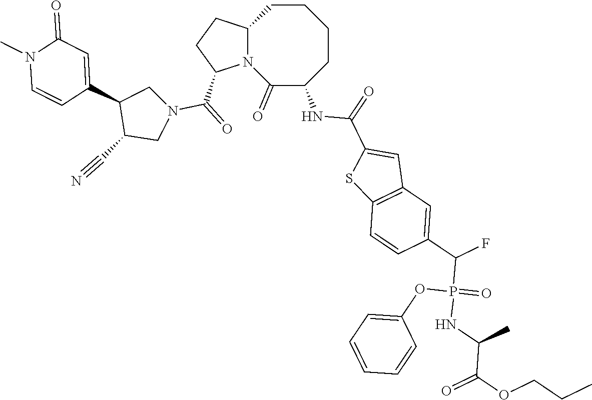 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@H](c6ccn(C)c(=O)c6)[C@@H](C#N)C5)N4C3=O)cc2c1] Pyrrolidine building block SFC peak 2 was used for synthesis and biological testing | | 1.47 (m, 4H), 1.22 (t, J = 6.5 Hz, 2H), 1.09- 1.03 (m, 1H), 0.93- 0.82 (m, 3H) |
| 319 | propyl (((2- (((3S,6S,10aS)-3- ((3S,4R)-3-cyano- 4-(1-methyl-2-oxo- 1,2-dihydropyridin- 4-yl)pyrrolidine-1- carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((2- (((3S,6S,10aS)-3- ((3R,4S)-3-cyano-4- (1-methyl-2-oxo- 1,2-dihydropyridin- 4-yl)pyrrolidine-1- carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 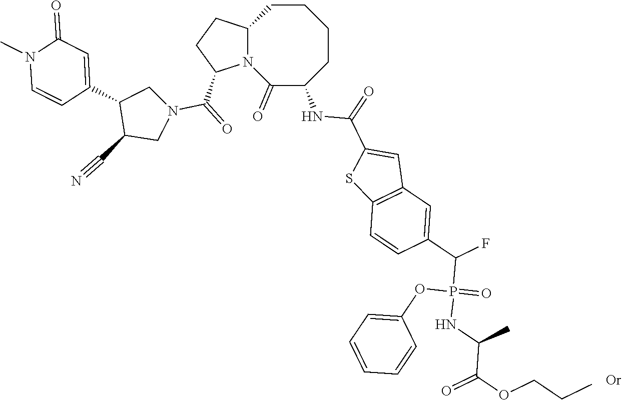 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@@H](C#N)[C@H](c6ccn(C)c(=O)c6)C5)N4C3=O)cc2c1] 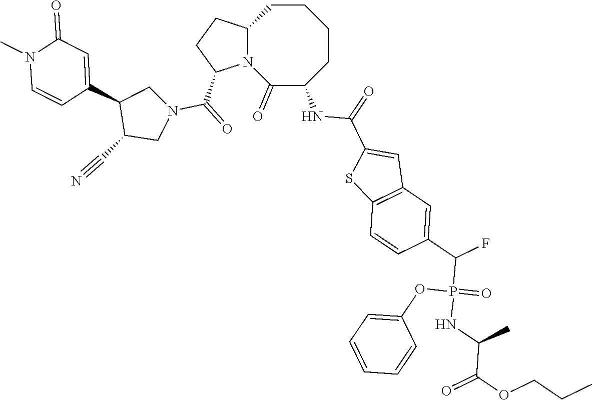 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@H](c6ccn(C)c(=O)c6)[C@@H](C#N)C5)N4C3=O)cc2c1] Pyrrolidine building block SFC peak 1 was used for synthesis and biological testing | 873.1 | ¹H NMR (400 MHz, CD₃OD) δ 8.14 (s, 1H), 8.08 (br. s., 1H), 8.03-7.93 (m, 1H), 7.70-7.60 (m, 2H), 7.38-7.29 (m, 2H), 7.25-7.12 (m, 3H), 6.62-6.54 (m, 1H), 6.46-6.40 (m, 1H), 6.20-5.98 (m, 1H), 5.07-4.97 (m, 1H), 4.72-4.61 (m, 1H), 4.57-4.37 (m, 2H), 4.10-3.77 (m, 4.5H), 3.76-3.45 (m, 6.5H), 2.42- 2.16 (m, 2H), 2.12- 1.92 (m, 6H), 1.91- 1.74 (m, 2H), 1.73- 1.46 (m, 4H), 1.26- 1.18 (m, 2H), 1.06 (d, J = 6.8 Hz, 1H), 0.93-0.82 (m, 3H) |
| 320 | propyl ((fluoro(2- (((3S,6S,10aS)-5- oxo-3-(3-(pyridin- 3-yl)pyrrolidine-1- carbonyl)decahydro pyrrolo[1,2- a]azocin-6- yl)carbamoyl)benzo [b]thiophen-5- yl)methyl)(phenoxy) phosphoryl)-L- alaninate | 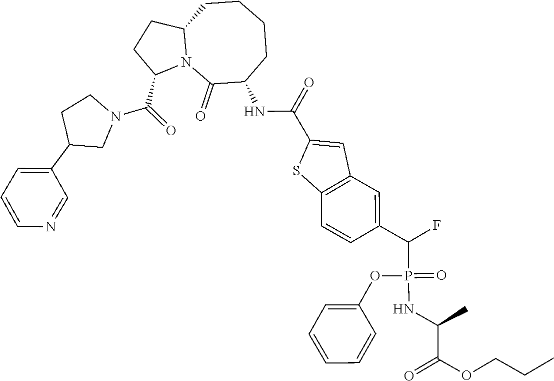 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CCC(c6cccnc6)C5)N4C3=O)cc2c1] | 818.1 | ¹H NMR (400 MHz, CD₃OD) δ 8.57- 8.46 (m, 1H), 8.45- 8.35 (m, 1H), 8.17- 8.10 (m, 1H), 8.10- 8.04 (m, 1H), 8.02- 7.91 (m, 1H), 7.87- 7.75 (m, 1H), 7.68- 7.59 (m, 1H), 7.45- 7.28 (m, 3H), 7.24- 7.12 (m, 3H), 6.20- 5.97 (m, 1H), 5.12- 5.00 (m, 1H), 4.78- 4.64 (m, 1H), 4.50- 4.34 (m, 1H), 4.18- 3.34 (m, 8H), 2.51- 1.93 (m, 10H), 1.91- 1.74 (m, 2H), 1.74- 1.44 (m, 4H), 1.26- 1.17 (m, 2H), 1.05 (d, J = 7.3 Hz,1H), 0.92-0.81 (m, 3H) |
| 321 | propyl (((S)-(2- (((3S,6S,10aS)-3- ((3S,4R)-3-cyano- 4. phenylpyrrolidine- 1-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((S)-(2- (((3S,6S,10aS)-3- ((3R,4S)-3-cyano-4- phenylpyrrolidine- 1-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 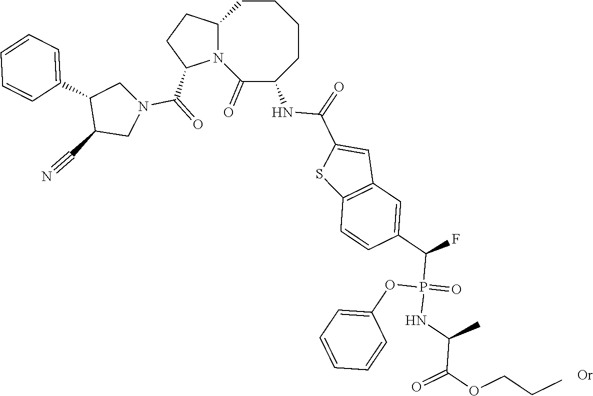 [SMILES: CCCOC(=O)[C@H](C)NP(=O)(Oc1ccccc1)[C@H](F)c1ccc2sc(C(=O)N[C@H]3CCCC[C@H]4CC[C@@H](C(=O)N5C[C@@H](C#N)[C@H](c6ccccc6)C5)N4C3=O)cc2c1] 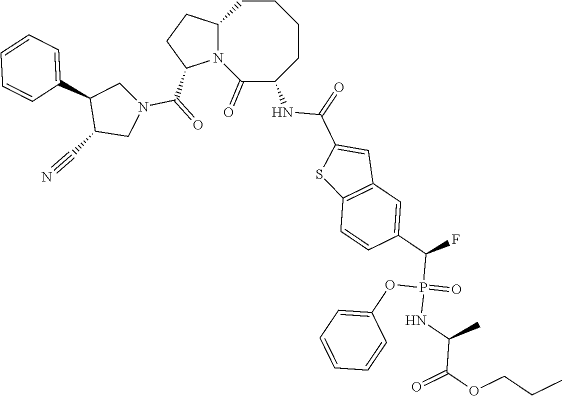 [SMILES: CCCOC(=O)[C@H](C)NP(=O)(Oc1ccccc1)[C@H](F)c1ccc2sc(C(=O)N[C@H]3CCCC[C@H]4CC[C@@H](C(=O)N5C[C@H](c6ccccc6)[C@@H](C#N)C5)N4C3=O)cc2c1] Pyrrolidine building block SFC peak 2 was used for synthesis and biological testing | 842.1 | ¹H NMR (400 MHz, CD₃OD) δ 8.16- 8.12 (m, 1H), 8.11- 8.06 (m, 1H), 8.03- 7.92 (m, 1H), 7.69- 7.60 (m, 1H), 7.43- 7.28 (m, 7H), 7.25- 7.14 (m, 3H), 6.19- 5.98 (m, 1H), 5.08- 4.99 (m, 1H), 4.73- 4.64 (m, 1H), 4.57- 4.38 (m, 2H), 4.11- 3.90 (m, 3H), 3.88- 3.64 (m, 3H), 3.63- 3.50 (m, 2H), 2.42- 2.16 (m, 2H), 2.12- 1.93 (m, 6H), 1.92- 1.74 (m, 2H), 1.73- 1.60 (m, 2H), 1.59- 1.48 (m, 2H), 1.23 and 1.06 (d, J = 7.1 Hz, 3H), 0.87 (t, J = 7.5 Hz, 3H) |
| 322 | propyl (((2- (((3S,6S,10aS)-3- ((rel-trans)-3- cyano-4-(2- methoxypyridin-3- yl)pyrrolidine-1- carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 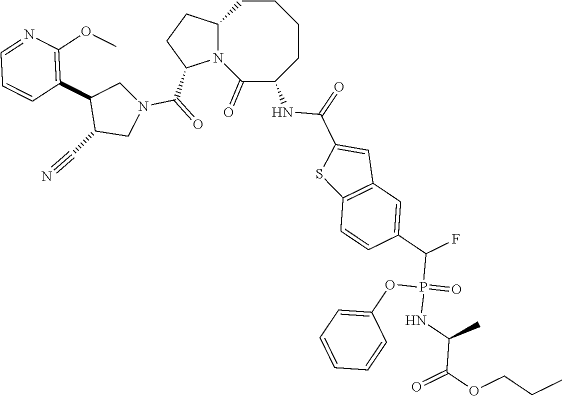 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@H](c6cccnc6OC)[C@@H](C#N)C5)N4C3=O)cc2c1] | 873.1 | ¹H NMR (400 MHz, CD₃OD) δ 8.17- 8.04 (m, 3H), 8.03- 7.93 (m, 1H), 7.72- 7.58 (m, 2H), 7.38- 7.28 (m, 2H), 7.24- 7.12 (m, 3H), 7.04- 6.93 (m, 1H), 6.21- 5.97 (m, 1H), 5.10- 4.98 (m, 1H), 4.73- 4.60 (m, 1H), 4.50- 4.35 (m, 1.5H), 4.25- 3.55 (m, 11.5H), 2.44-2.17 (m, 2H), 2.11-1.92 (m, 6H), 1.91-1.75 (m, 2H), 1.74-1.45 (m, 4H), 1.27-1.17 (m, 2H), 1.09-1.03 (m, 1H), 0.92-0.82 (m, 3H) |
| 323 | butyl (2S)-1- ((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-4,4- difluoropyrrolidine- 2-carboxylate | 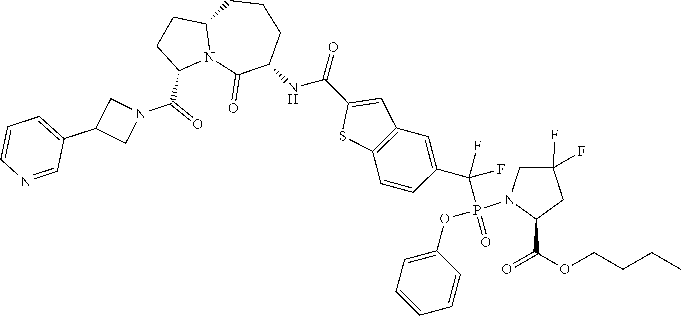 [SMILES: CCCCOC(=O)[C@@H]1CC(F)(F)CN1P(=O)(Oc1ccccc1)C(F)(F)c1ccc2sc(C(=O)N[C@H]3CCC[C@H]4CC[C@@H](C(=O)N5CC(c6cccnc6)C5)N4C3=O)cc2c1] | 884.1 | ¹H NMR (400 MHz, CD₃OD) δ 8.64- 8.37 (m, 2H), 8.28- 7.93 (m, 4H), 7.76- 7.71 (m, 1H), 7.51- 7.39 (m, 1H), 7.37- 7.28 (m, 2H), 7.23- 7.14 (m, 1H), 7.14- 7.08 (m, 2H), 4.98- 4.92 (m, 0.5H), 4.80- 7.74 (m, 1.5H), 4.75-4.68 (m, 1H), 4.64-4.28 (m, 3H), 4.13-3.95 (m, 5H), 3.75-3.65 (m, 2H), 2.86-2.48 (m, 3H), 2.38-2.27 (m, 1H), 2.19-2.05 (m, 2H), 2.05-1.93 (m, 3H), 1.93-1.81 (m, 3H), 1.57-1.49 (m, 2H), 1.38-1.28 (m, 2H), 0.93-0.85 (m, 3H) |
| 324 | S,S′-((((difluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)phosphoryl) bis(oxy))bis(ethane- 2,1-diyl)) bis(2,2- dimethyl- propanethioate) | 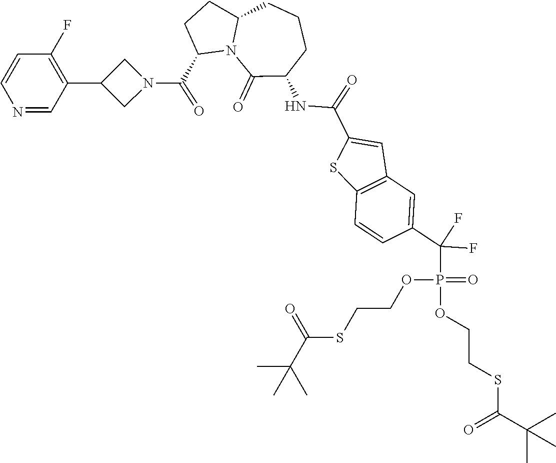 [SMILES: CC(C)(C)C(=O)SCCOP(=O)(OCCSC(=O)C(C)(C)C)C(F)(F)c1ccc2sc(C(=O)N[C@H]3CCC[C@H]4CC[C@@H](C(=O)N5CC(c6cnccc6F)C5)N4C3=O)cc2c1] | 925.1 | ¹H NMR (400 MHz, CD₃OD) δ 8.81- 8.58 (m, 1H), 8.56- 8.45 (m, 1H), 8.23- 8.15 (m, 2H), 8.14- 8.05 (m, 1H), 7.71- 7.59 (m, 1H), 7.32- 7.18 (m, 1H), 4.99- 4.89 (m, 1H), 4.80- 4.69 (m, 2H), 4.65- 4.38 (m, 2H), 4.30- 4.02 (m, 7H), 3.22- 3.05 (m, 4H), 2.38- 2.24 (m, 1H), 2.20- 1.75 (m, 9H), 1.25- 1.13 (m, 18H) |
| 325 | propyl (((2- (((3S,6S,10aS)-3- ((rel-trans)-7- cyano-6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 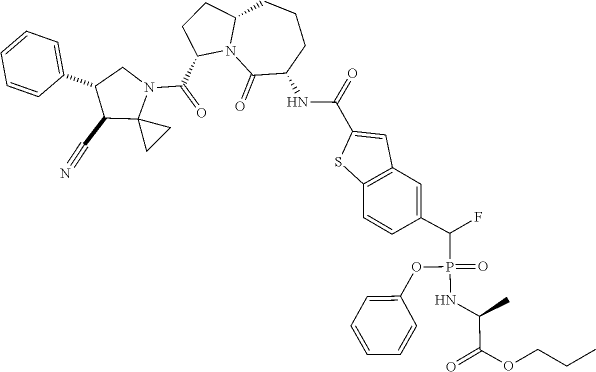 [SMILES: CCCOC(=O)[C@H](C)NP(=O)(Oc1ccccc1)C(F)c1ccc2sc(C(=O)N[C@H]3CCC[C@H]4CC[C@@H](C(=O)N5C[C@@H](c6ccccc6)[C@H](C#N)C56CC6)N4C3=O)cc2c1] | 868.1 | ¹H NMR (400 MHz, CD₃OD) δ 8.16- 8.11 (m, 1H), 8.10- 8.04 (m, 1H), 8.02- 7.92 (m, 1H), 7.69- 7.60 (m, 1H), 7.51- 7.44 (m, 1H), 7.43- 7.25 (m, 6H), 7.24- 7.12 (m, 3H), 6.19- 5.98 (m, 1H), 5.06- 4.96 (m, 1H), 4.67- 4.54 (m, 1H), 4.45- 4.33 (m, 1H), 4.21- 4.07 (m, 1H), 4.02- 3.63 (m, 6H), 2.42- 2.13 (m, 3H), 2.08- 1.72 (m, 9H), 1.70- 1.44 (m, 4H), 1.27- 1.15 (m, 2H), 1.11- 1.02 (m, 1H), 1.02- 0.93 (m, 1H), 0.92- 0.81 (m, 3H), 0.77- 0.62 (m, 1H). |
| 326 | propyl (((2- (((3S,6S,10aS)-3- ((6R,7S)-7-cyano- 6-(3- methoxyphenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((2- (((3S,6S,10aS)-3- ((6S,7R)-7-cyano-6- (3-methoxyphenyl)- 4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 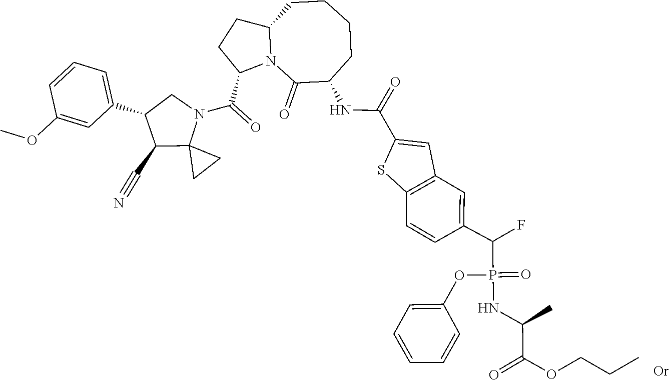 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@@H](c6cccc(OC)c6)[C@H](C#N)C56CC6)N4C3=O)cc2c1] 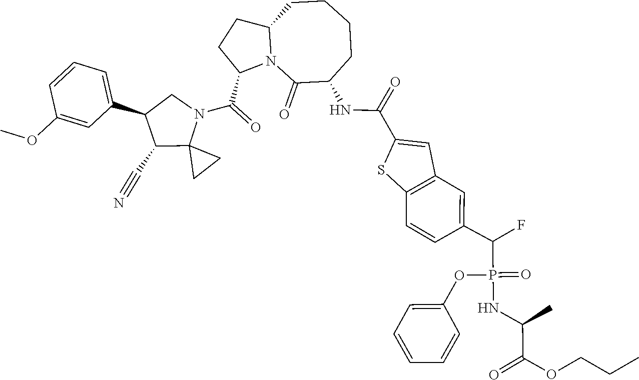 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@H](c6cccc(OC)c6)[C@@H](C#N)C56CC6)N4C3=O)cc2c1] | 898.1 | ¹H NMR (400 MHz, CD₃OD) δ 8.15- 8.10 (m, 1H), 8.10- 8.05 (m, 1H), 8.02- 7.91 (m, 1H), 7.69- 7.58 (m, 1H), 7.40- 7.12 (m, 6H), 7.06- 7.00 (m, 1H), 7.00- 6.94 (m, 1H), 6.91- 6.81 (m, 1H), 6.21- 5.96 (m, 1H), 5.06- 4.97 (m, 1H), 4.67- 4.56 (m, 1H), 4.45- 4.33 (m, 1H), 4.20- 4.07 (m, 1H), 4.02- 3.64 (m, 9H), 2.42- 2.14 (m, 3H), 2.07- 1.72 (m, 9H), 1.70- 1.46 (m, 4H), 1.27- 1.19 (m, 2H), 1.09- 1.03 (m, 1H), 1.02- 0.93 (m, 1H), 0.92- 0.82 (m, 3H), 0.78- 0.65 (m, 1H) |
| 327 | propyl (((R)-(2- (((3S,6S,10aS)-3- (5,7-dioxo-6-(6- (trifluoromethyl) pyridin-3-yl)-4,6- diazaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 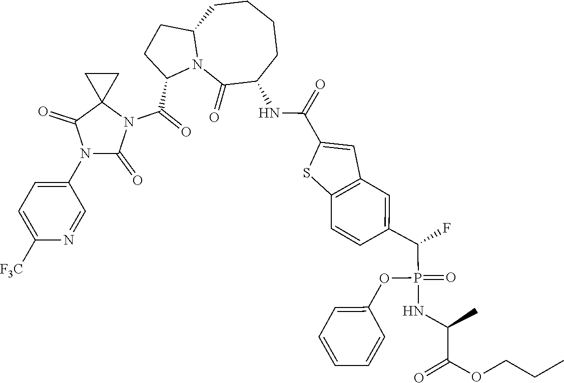 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6ccc(C(F)(F)F)nc6)C(=O)C56CC6)N4C3=O)cc2c1] | 941.0 | ¹H NMR (400 MHz, CD₃OD) δ 8.91- 8.87 (m, 1H), 8.24- 8.18 (m, 1H), 8.12 (s, 1H), 8.06 (s, 1H), 8.00-7.93 (m, 2H), 7.66-7.58 (m, 1H), 7.37-7.28 (m, 2H), 7.24-7.12 (m, 3H), 6.16-5.98 (m, 1H), 5.61-5.55 (m, 1H), 5.09-5.01 (m, 1H), 4.53-4.43 (m, 1H), 3.99-3.78 (m, 3H), 2.63-2.54 (m, 1H), 2.47-2.27 (m, 3H), 2.10-1.42 (m, 14H), 1.24-1.15 (m, 3H), 0.86 (dt, J = 10.0, 7.5 Hz, 3H) |
| 328 | propyl (((R)- fluoro(2- (((3S,6S,10aS)-3- (6-(6- methylpyridin-3- yl)-5,7-dioxo-4,6- diazaspiro[2.4]heptane- 4-carbonyl)-5- oxodecahydropyrrolo [1,2-a]azocin-6- yl)carbamoyl)benzo [b]thiophen-5- yl)methyl)(phenoxy) phosphoryl)-L- alaninate | 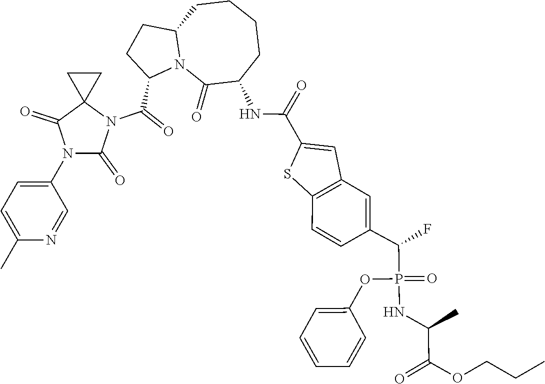 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6ccc(C)nc6)C(=O)C56CC6)N4C3=O)cc2c1] | 887.1 | ¹H NMR (400 MHz, CD₃OD) δ 8.61- 8.49 (m, 1H), 8.16- 8.05 (m, 2H), 8.01- 7.94 (m, 1H), 7.88- 7.82 (m, 1H), 7.68- 7.60 (m, 1H), 7.48- 7.42 (m, 1H), 7.37- 7.29 (m, 2H), 7.24- 7.13 (m, 3H), 6.18- 5.99 (m, 1H), 5.61- 5.53 (m, 1H), 5.09- 5.00 (m, 1H), 4.53- 4.44 (m, 1H), 4.01- 3.77 (m, 3H), 2.58- 2.52 (m, 1H), 2.45- 2.26 (m, 3H), 2.10- 1.78 (m, 9H), 1.77- 1.63 (m, 2H), 1.62- 1.39 (m, 5H), 1.31- 1.10 (m, 4H), 0.91- 0.82 (m, 3H) |
| 329 | propyl (((R)- fluoro(2- (((3S,6S,10aS)-3- (6-(6- methoxypyridin-3- yl)-5,7-dioxo-4,6- diazaspiro[2.4]heptane- 4-carbonyl)-5- oxodecahydropyrrolo [1,2-a]azocin-6- yl)carbamoyl)benzo [b]thiophen-5- yl)methyl)(phenoxy) phosphoryl)-L- alaninate | 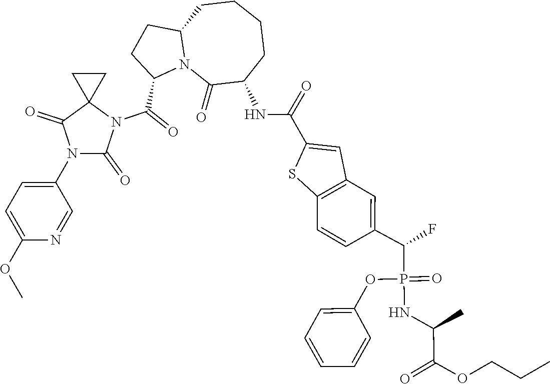 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6ccc(OC)nc6)C(=O)C56CC6)N4C3=O)cc2c1] | 903.1 | ¹H NMR (400 MHz, CD₃OD) δ 8.23- 8.18 (m, 1H), 8.11 (s, 1H), 8.07 (s, 1H), 8.00-7.94 (m, 1H), 7.76-7.70 (m, 1H), 7.67-7.59 (m, 1H), 7.38-7.28 (m, 2H), 7.24-7.13 (m, 3H), 6.92-6.86 (m, 1H), 6.17-5.98 (m, 1H), 5.60-5.54 (m, 1H), 5.08-5.00 (m, 1H), 4.52-4.41 (m, 1H), 4.01-3.78 (m, 6H), 2.61-2.49 (m, 1H), 2.45-2.23 (m, 3H), 2.12-1.37 (m, 14H), 1.25-1.17 (m, 3H), 0.86 (dt, J = 10.4, 7.4 Hz, 3H) |
| 330 | propyl (((R)-(2- (((3S,6S,10aS)-3- (5,7-dioxo-6-(5- (trifluoromethyl) pyridin-3-yl)-4,6- diazaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 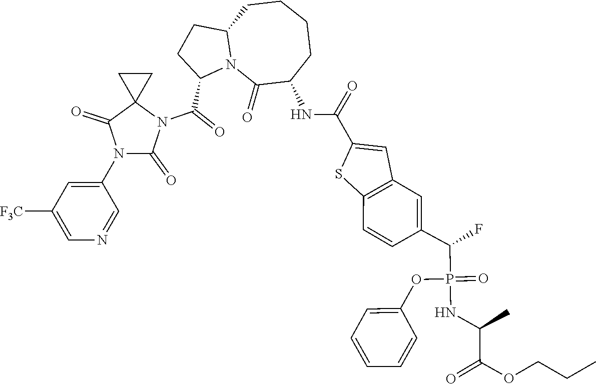 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6cncc(C(F)(F)F)c6)C(=O)C56CC6)N4C3=O)cc2c1] | 941.0 | ¹H NMR (400 MHz, CD₃OD) δ 9.00- 8.96 (m, 1H), 8.95- 8.93 (m, 1H), 8.34 (s, 1H), 8.12 (s, 1H), 8.06 (s, 1H), 7.99- 7.93 (m, 1H), 7.66- 7.56 (m, 1H), 7.38- 7.28 (m, 2H), 7.23- 7.12 (m, 3H), 6.17- 5.97 (m, 1H), 5.61- 5.55 (m, 1H), 5.09- 5.01 (m, 1H), 4.53- 4.43 (m, 1H), 4.01- 3.77 (m, 3H), 2.64- 2.54 (m, 1H), 2.47- 2.39 (m, 1H), 2.39- 2.27 (m, 2H), 2.10- 1.78 (m, 8H), 1.76- 1.42 (m, 6H), 1.23- 1.17 (m, 3H), 0.86 (dt, J = 10.1, 7.5 Hz, 3H) |
| 331 | propyl (((R)- fluoro(2- (((3S,6S,10aS)-3- (6-(5- methoxypyridin-3- yl)-5,7-dioxo-4,6- diazaspiro[2.4]heptane- 4-carbonyl)-5- oxodecahydropyrrolo [1,2-a]azocin-6- yl)carbamoyl)benzo [b]thiophen-5- yl)methyl)(phenoxy) phosphoryl)-L- alaninate | 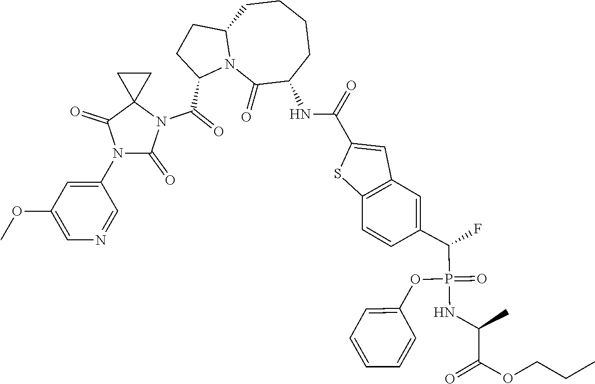 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6cncc(OC)c6)C(=O)C56CC6)N4C3=O)cc2c1] | 903.2 | ¹H NMR (400 MHz, CD₃OD) δ 8.33- 8.23 (m, 2H), 8.11 (s, 1H), 8.06 (s, 1H), 7.99-7.93 (m, 1H), 7.67-7.55 (m, 2H), 7.37-7.28 (m, 2H), 7.24-7.11 (m, 3H), 6.16-5.98 (m, 1H), 5.63-5.53 (m, 1H), 5.08-5.00 (m, 1H), 4.52-4.42 (m, 1H), 4.01-3.76 (m, 6H), 2.62-2.52 (m, 1H), 2.46-2.26 (m, 3H), 2.10-1.79 (m, 8H), 1.75-1.39 (m, 6H), 1.24-1.16 (m, 3H), 0.86 (dt, J = 10.0, 7.4 Hz, 3H) |
| 332 | propyl (((R)-(2- (((3S,6S,10aS)-3- (6-(6- (difluoromethyl) pyridin-3-yl)-5,7- dioxo-4,6- diazaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 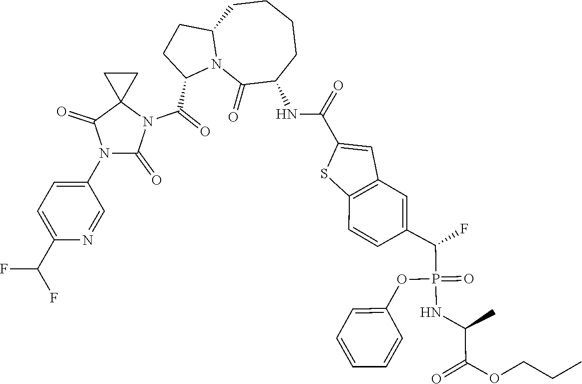 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6ccc(C(F)F)nc6)C(=O)C56CC6)N4C3=O)cc2c1] | 923.3 | ¹H NMR (400 MHz, CD₃OD) δ 8.82- 8.78 (m, 1H), 8.18- 8.11 (m, 2H), 8.07 (s, 1H), 8.00-7.94 (m , 1H), 7.87-7.81 (m, 1H), 7.67-7.59 (m, 1H), 7.37-7.28 (m, 2H), 7.24-7.12 (m, 3H), 6.93-6.62 (m, 1H), 6.17-5.98 (m, 1H), 5.63-5.53 (m, 1H), 5.09-5.01 (m, 1H), 4.53-4.43 (m, 1H), 4.01-3.77 (m, 3H), 2.63-2.53 (m, 1H), 2.47-2.27 (m, 3H), 2.11-1.77 (m, 8H), 1.77-1.41 (m, 6H), 1.23-1.17 (m, 3H), 0.86 (dt, J = 10.3, 7.5 Hz, 3H) |
| 333 | propyl (((R)-(2- (((3S,6S,10aS)-3- (6-(5- (difluoromethyl) pyridin-3-yl)-5,7- dioxo-4,6- diazaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 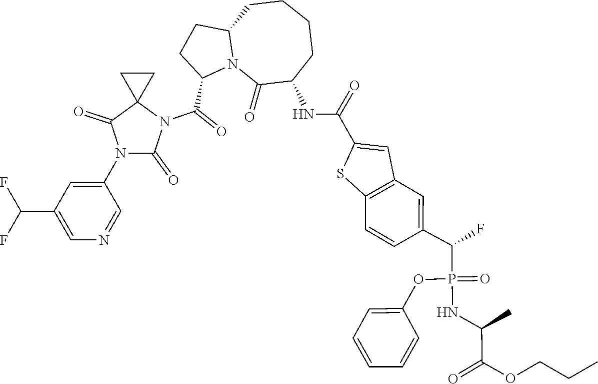 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6cncc(C(F)F)c6)C(=O)C56CC6)N4C3=O)cc2c1] | 923.2 | ¹H NMR (400 MHz, CD₃OD) δ 8.86 (s, 1H), 8.77 (s, 1H), 8.19 (s, 1H), 8.12 (s, 1H), 8.07 (s, 1H), 8.00-7.93 (m, 1H), 7.66-7.59 (m, 1H), 7.39-7.27 (m, 2H), 7.24-7.13 (m, 3H), 7.13-6.83 (m, 1H), 6.16-5.98 (m, 1H), 5.62-5.54 (m, 1H), 5.09-5.00 (m, 1H), 4.54-4.42 (m, 1H), 4.01-3.76 (m, 3H), 2.63-2.53 (m, 1H), 2.47-2.39 (m, 1H), 2.39-2.27 (m, 2H), 2.11-1.79 (m, 8H), 1.77-1.42 (m, 6H), 1.24-1.16 (m, 3H), 0.86 (dt, J = 10.4, 7.4 Hz, 3H) |
| 334 | propyl (((R)- fluoro(2- (((3S,6S,10aS)-3- (6-(4- methylpyridin-3- yl)-5,7-dioxo-4,6- diazaspiro[2.4]heptane- 4-carbonyl)-5- oxodecahydropyrrolo [1,2-a]azocin-6- yl)carbamoyl)benzo [b]thiophen-5- yl)methyl)(phenoxy) phosphoryl)-L- alaninate | 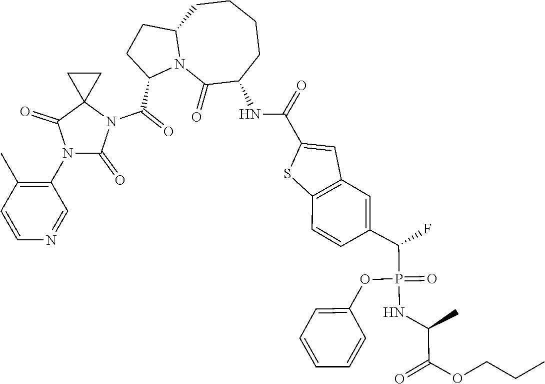 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6cnccc6C)C(=O)C56CC6)N4C3=O)cc2c1] | 887.1 | ¹H NMR (400 MHz, CD₃OD) δ 8.53- 8.46 (m, 1H), 8.12 (s, 1H), 8.08 (br. s., 1H), 8.02-7.95 (m, 1H), 7.67-7.60 (m, 1H), 7.52-7.45 (m, 1H), 7.37-7.31 (m, 1H), 7.24-7.02 (m, 3H), 6.86-6.70 (m, 1H), 6.15-5.99 (m, 1H), 5.59-5.49 (m, 1H), 5.07-5.00 (m, 1H), 4.61-4.55 (m, 1H), 4.52-4.43 (m, 1H), 4.00-3.95 (m, 1H), 3.87-3.80 (m, 1H), 3.77-3.69 (m, 1H), 3.26-3.20 (m, 1H), 2.31-2.29 (m, 2H), 2.06-1.99 (m, 3H), 1.88-1.82 (m, 1H), 1.62-1.59 (m, 1H), 1.58-1.54 (m, 1H), 1.50-1.43 (m, 2H), 1.39-1.35 (m, 7H), 1.31-1.28 (m, 3H), 1.23-1.20 (m, 2H), 0.92-0.83 (m, 4H) |
| 335 | propyl (((R)-(2- (((3S,6S,10aS)-3- ((6S,7R)-7-cyano- 6-(3- methoxyphenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S, 10aS)-3- ((6R,7S)-7-cyano-6- (3-methoxyphenyl)- 4- azaspiro[2.4]heptane- 4-carbonyl)-5- oxodecahydropyrrolo | 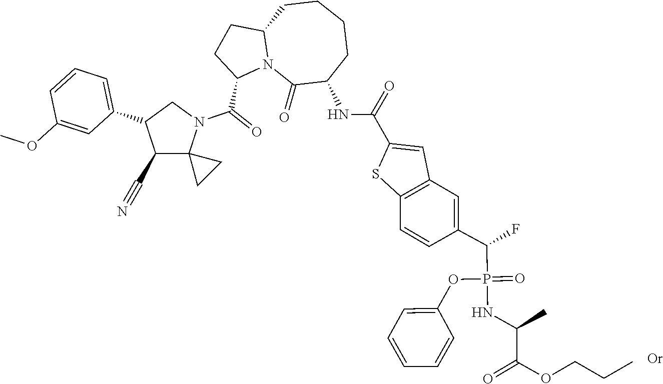 | 898.1 | ¹H NMR (400 MHz, CD₃OD) δ 8.15- 8.09 (m, 1H), 8.09- 8.04 (m, 1H), 7.99- 7.93 (m, 1H), 7.67- 7.59 (m, 1H), 7.39- 7.28 (m, 2H), 7.28- 7.12 (m, 4H), 7.07- 6.99 (m, 2H), 6.88- 6.79 (m, 1H), 6.20- 5.96 (m, 1H), 5.05- 4.96 (m, 1H), 4.63- 4.51 (m, 1H), 4.45- 4.33 (m, 1H), 4.20- 4.07 (m, 2H), 3.97 (t, J = 6.6 Hz, 1H), 3.95- 3.75 (m, 3H), 3.75 (s, 3H), 3.73-3.63 (m, 2H), 2.44-2.27 (m, 2H), 2.26-2.13 (m, 1H), 2.10-1.87 (m, 6H), 1.86-1.71 (m, 3H), 1.70-1.44 |
| | [1,2-a]azocin-6- yl)carbamoyl)benzo [b]thiophen-5- yl)fluoromethyl) (phenoxy)phosphoryl)- L-alaninate | 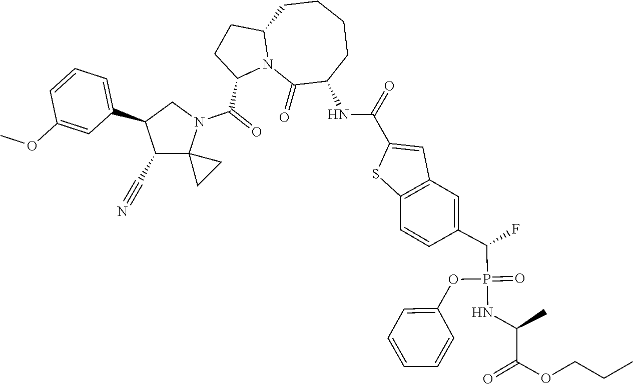 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccc(OC)c6)[C@@H](C#N)C56CC6)N4C3=O)cc2c1] Pyrrolidine + Core SFC peak 1 was used for synthesis and biological testing | | (m, 4H), 1.21 (d, J = 7.1 Hz, 3H), 1.02- 0.92 (m, 1H), 0.92- 0.81 (m, 3H), 0.77- 0.62 (m, 1H) |
| 336 | propyl (((R)-(2- (((3S,6S,10aS)-3- ((6S,7R)-7-cyano- 6-(3- methoxyphenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6R,7S)-7-cyano-6- (3-methoxyphenyl)- 4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 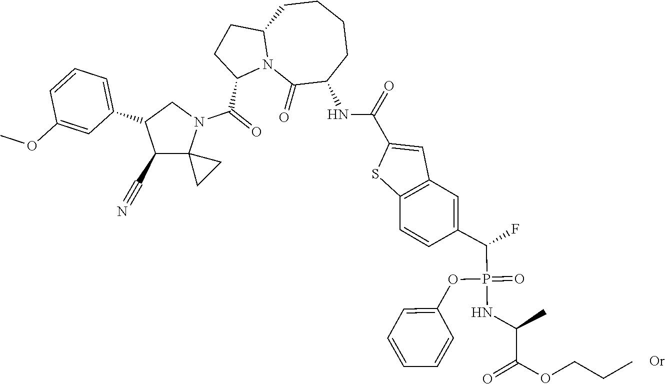 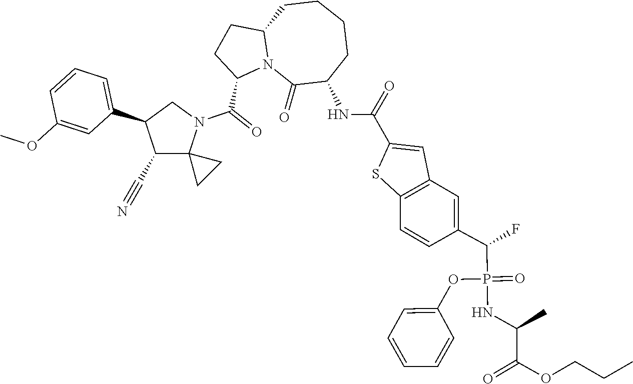 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccc(OC)c6)[C@@H](C#N)C56CC6)N4C3=O)cc2c1] Pyrrolidine + Core SFC peak 2 was used for synthesis and biological testing | 898.1 | ¹H NMR (400 MHz, CD₃OD) δ 8.13- 8.10 (m, 1H), 8.08- 8.05 (m, 1H), 8.00- 7.90 (m, 1H), 7.68- 7.58 (m, 1H), 7.41- 7.10 (m, 6H), 7.03- 6.93 (m, 2H), 6.91- 6.83 (m, 1H), 6.19- 5.97 (m, 1H), 5.07- 4.97 (m, 1H), 4.67- 4.51 (m, 2H), 4.45- 4.32 (m, 1H), 3.98 (t, J = 6.6 Hz, 1H), 3.94- 3.79 (m, 3H), 3.77 (s, 3H), 3.74-3.65 (m, 2H), 2.37-2.12 (m, 3H), 2.09-1.87 (m, 7H), 1.87-1.73 (m, 2H), 1.70-1.42 (m, 4H), 1.21 (d, J = 7.1 Hz, 3H), 1.03- 0.93 (m, 1H), 0.92- 0.82 (m, 3H), 0.79- 0.65 (m, 1H) |
| 337 | propyl (((R)-(2- (((3S,6S,10aS)-3- ((rel-trans)-7- cyano-6-(3- fluorophenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 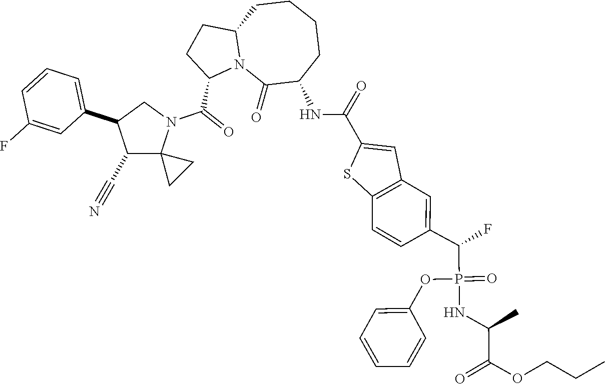 | 886.1 | ¹H NMR (400 MHz, CD₃OD) δ 8.15- 8.12 (m, 1H), 8.10- 8.06 (m, 1H), 8.00- 7.94 (m, 1H), 7.67- 7.60 (m, 1H), 7.43- 7.13 (m, 8H), 7.10- 7.00 (m, 1H), 6.17- 6.01 (m, 1H), 5.06- 4.98 (m, 1H), 4.63- 4.55 (m, 1H), 4.44- 4.35 (m, 1H), 4.20- 4.08 (m, 1H), 4.00- 3.96 (m, 1H), 3.95- 3.67 (m, 5H), 2.41- 2.14 (m, 3H), 2.07- 1.89 (m, 6H), 1.87- 1.74 (m, 2H), 1.70- 1.45 (m, 4H), 1.37- 1.27 (m, 1H), 1.24- 1.19 (m, 3H), 1.02- 0.93 (m, 1H), 0.92- 0.82 (m, 3H), 0.77- 0.65 (m, 1H) |
| 338 | propyl (((R)-(2- (((3S,6S,10aS)-3- ((rel-trans)-7- cyano-6-(3- (difluoromethoxy) phenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 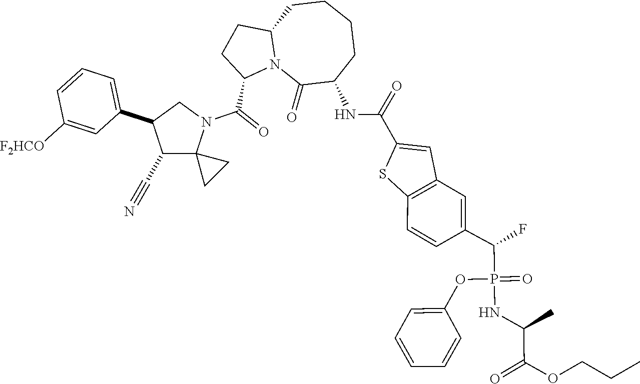 | 934.1 | ¹H NMR (400 MHz, CD₃OD) δ 8.18- 8.04 (m, 2H), 8.01- 7.91 (m, 1H), 7.69- 7.57 (m, 1H), 7.45- 7.27 (m, 5H), 7.26- 7.08 (m, 4H), 7.06- 6.62 (m, 1H), 6.20- 5.97 (m, 1H), 5.06- 4.96 (m, 1H), 4.63- 4.56 (m, 1H), 4.46- 4.33 (m, 1H), 4.23- 4.11 (m, 1H), 3.98 (t, J = 6.6 Hz, 1H), 3.89-3.63 (m, 4H), 2.41-2.14 (m, 3H), 2.08-1.88 (m, 7H), 1.86-1.42 (m, 7H), 1.21 (d, J = 7.3 Hz, 3H), 1.02-0.92 (m, 1H), 0.91-0.81 (m, 3H), 0.77-0.63 (m, 1H) |
| 339 | propyl (((R)-(2- (((3S,6S,10aS)-3- ((6S,7R)-7-cyano- 6-(3-cyanophenyl)- 4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6R,7S)-7-cyano-6- (3-cyanophenyl)-4- azaspiro[2.4]heptane- 4-carbonyl)-5- oxodecahydropyrrolo [1,2-a]azocin-6- | 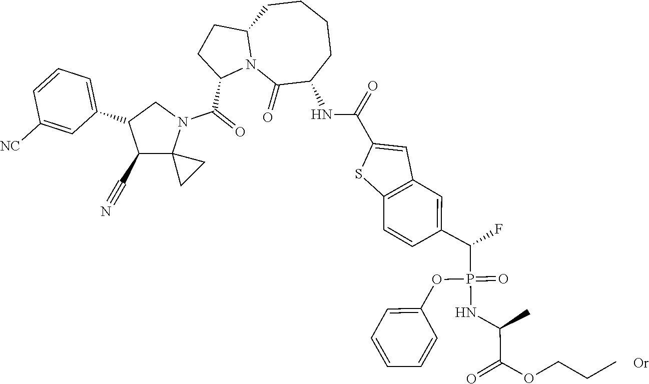 | 893.1 | ¹H NMR (400 MHz, CD₃OD) δ 8.13 (s, 1H), 8.09-8.05 (m, 1H), 8.01-7.93 (m, 1H), 7.88-7.80 (m, 1H), 7.78-7.67 (m, 2H), 7.66-7.59 (m, 1H), 7.59-7.53 (m, 1H), 7.39-7.29 (m, 2H), 7.25-7.12 (m, 3H), 6.19-5.99 (m, 1H), 5.07-4.97 (m, 1H), 4.67-4.54 (m, 2H), 4.45-4.33 (m, 1H), 3.98 (t, J = 6.6 Hz, 1H), 3.94-3.73 (m, 5H), 2.37-2.12 (m, 3H), 2.07-1.88 (m, 7H), 1.86-1.72 (m, 2H), 1.71-1.47 (m, 4H), 1.21 (d, J = 7.1 Hz, 3H), 1.04- 0.94 (m, 1H), 0.94- |
| | yl)carbamoyl)benzo [b]thiophen-5- yl)fluoromethyl) (phenoxy)phosphoryl)- L-alaninate | 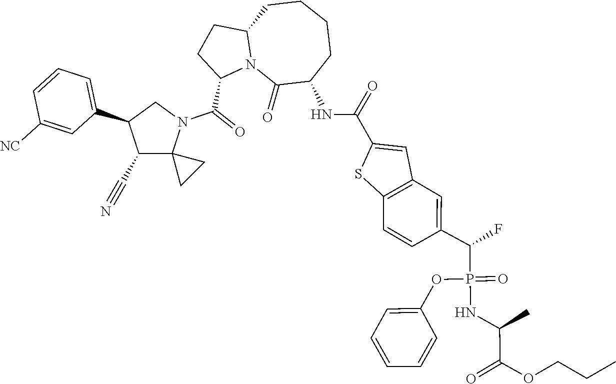 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccc(C#N)c6)[C@@H](C#N)C56CC6)N4C3=O)cc2c1] Pyrrolidine + Core SFC peak 1 was used for synthesis and biological testing | | 0.82 (m, 3H), 0.79- 0.68 (m, 1H) |
| 340 | propyl (((R)-(2- (((3S,6S,10aS)-3- ((6S,7R)-7-cyano- 6-(3-cyanophenyl)- 4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6R,7S)-7-cyano-6- (3-cyanophenyl)-4- azaspiro[2.4]heptane- 4-carbonyl)-5- oxodecahydropyrrolo [1,2-a]azocin-6- | 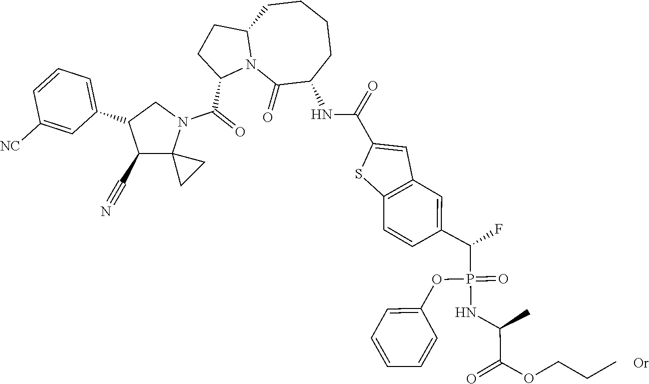 | 893.1 | ¹H NMR (400 MHz, CD₃OD) δ 8.14 (s, 1H), 8.10-8.06 (m, 1H), 8.01-7.94 (m, 2H), 7.87-7.79 (m, 1H), 7.69-7.60 (m, 2H), 7.58-7.52 (m, 1H), 7.39-7.29 (m, 2H), 7.25-7.13 (m, 3H), 6.18-5.99 (m, 1H), 5.07-4.99 (m, 1H), 4.65-4.57 (m, 1H), 4.45-4.35 (m, 1H), 4.28-4.16 (m, 2H), 3.98 (t, J = 6.6 Hz, 1H), 3.94-3.77 (m, 3H), 3.65 (d, J = 8.8 Hz, 1H), 2.39- 2.13 (m, 3H), 2.09- 1.90 (m, 6H), 1.89- 1.72 (m, 3H), 1.70- 1.44 (m, 4H), 1.21 (d, J = 7.1 Hz, 3H), |
| | yl)carbamoyl)benzo [b]thiophen-5- yl)fluoromethyl) (phenoxy)phosphoryl)- L-alaninate | 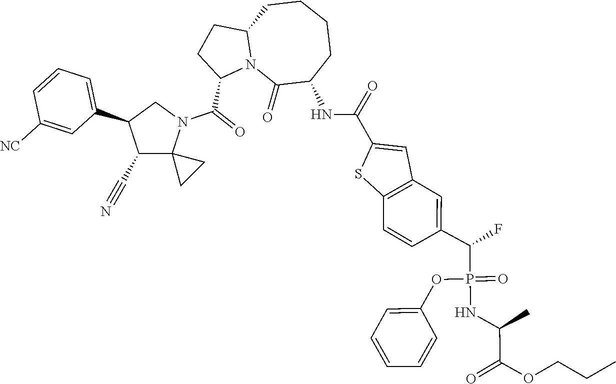 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccc(C#N)c6)[C@@H](C#N)C56CC6)N4C3=O)cc2c1] Pyrrolidine + Core SFC peak 2 was used for synthesis and biological testing | | 0.98-0.91 (m, 1H), 0.90-0.82 (m, 3H), 0.70-0.60 (m, 1H) |
| 341 | propyl (((R)-(2- (((3S,6S,9aS)-3- ((6S,7R)-7-cyano- 6-(m-tolyl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-7-cyano- 6-(m-tolyl)-4- azaspiro[2.4]heptane- 4-carbonyl)-5- oxooctahydro-1H- pyrrolo[1,2- | 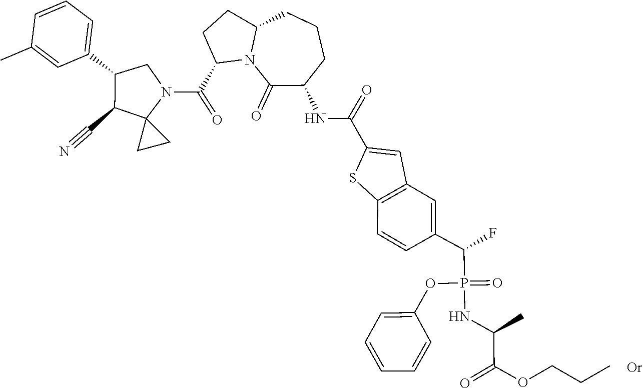 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cccc(C)c6)[C@H](C#N)C56CC6)N4C3=O)cc2c1] | 868.1 | ¹H NMR (400 MHz, CD₃OD) δ 8.13- 8.06 (m, 2H), 8.03- 7.93 (m, 1H), 7.70- 7.60 (m, 1H), 7.39- 7.30 (m, 3H), 7.27- 7.07 (m, 6H), 6.19- 5.99 (m, 1H), 4.77- 4.70 (m, 1H), 4.70- 4.64 (m, 1H), 4.18- 4.09 (m, 1H), 4.08- 4.01 (m, 2H), 3.98 (t, J = 6.6 Hz, 1H), 3.94-3.76 (m, 2H), 3.76-3.65 (m, 2H), 2.34 (s, 3H), 2.33- 2.24 (m, 2H), 2.23- 2.13 (m, 1H), 2.12- 1.74 (m, 9H), 1.63- 1.45 (m, 2H), 1.25- 1.19 (m, 3H), 1.03- 0.95 (m, 1H), 0.92- 0.83 (m, 3H), 0.74- |
| | a]azepin-6- yl)carbamoyl)benzo [b]thiophen-5- yl)fluoromethyl) (phenoxy)phosphoryl)- L-alaninate | 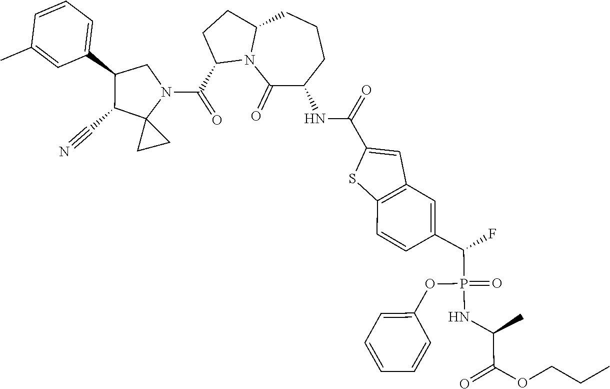 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6cccc(C)c6)[C@@H](C#N)C56CC6)N4C3=O)cc2c1] Pyrrolidine + Core SFC peak 1 was used for synthesis and biological testing | | 0.65 (m, 1H) |
| 342 | propyl (((R)-(2- (((3S,6S,9aS)-3- ((6S,7R)-7-cyano- 6-(m-tolyl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-7-cyano- 6-(m-tolyl)-4- azaspiro[2.4]heptane- 4-carbonyl)-5- oxooctahydro-1H- pyrrolo[1,2- | 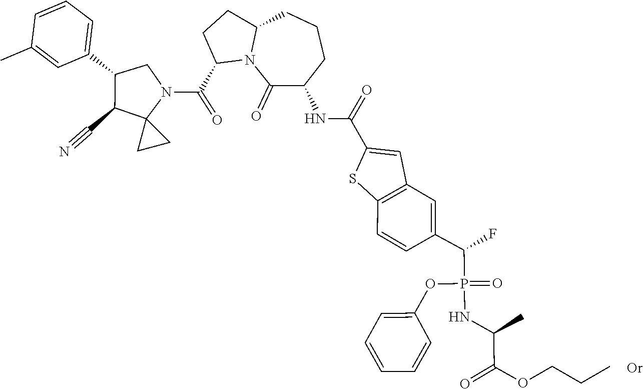 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cccc(C)c6)[C@H](C#N)C56CC6)N4C3=O)cc2c1] | 868.1 | ¹H NMR (400 MHz, CD₃OD) δ 8.14- 8.07 (m, 2H), 8.02- 7.96 (m, 1H), 7.70- 7.61 (m, 1H), 7.39- 7.11 (m, 9H), 6.20- 5.99 (m, 1H), 4.78- 4.66 (m, 2H), 4.50- 4.38 (m, 1H), 4.08- 3.79 (m, 5H), 3.78- 3.66 (m, 2H), 2.36 (s, 3H), 2.34-2.21 (m, 2H), 2.17-1.92 (m, 4H), 1.91-1.74 (m, 6H), 1.63-1.45 (m, 2H), 1.25-1.17 (m, 3H), 1.04-0.97 (m, 1H), 0.94-0.81 (m, 3H), 0.79-0.68 (m, 1H) |
| | a]azepin-6- yl)carbamoyl)benzo [b]thiophen-5- yl)fluoromethyl) (phenoxy)phosphoryl)- L-alaninate | 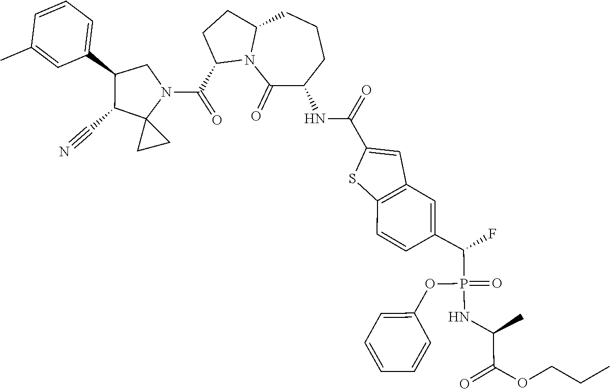 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6cccc(C)c6)[C@@H](C#N)C56CC6)N4C3=O)cc2c1] Pyrrolidine + Core SFC peak 2 was used for synthesis and biological testing | | |
| 343 | propyl (((R)-(2- (((3S,6S,10aS)-3- ((3R,4S)-3-cyano- 4- phenylpyrrolidine- 1-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((3S,4R)-3-cyano-4- phenylpyrrolidine- 1-carbonyl)-5- oxodecahydropyrrolo [1,2-a]azocin-6- yl)carbamoyl)benzo [b]thiophen-5- | 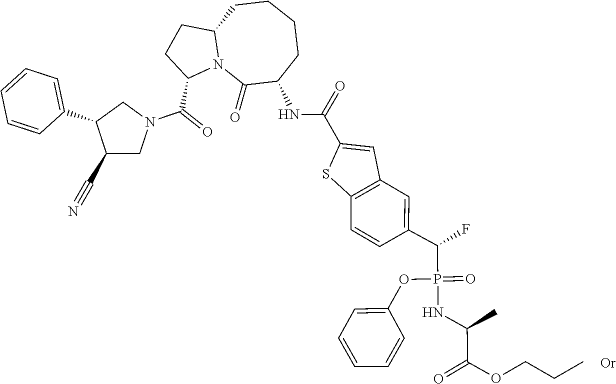 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](C#N)[C@H](c6ccccc6)C5)N4C3=O)cc2c1] | 842.1 | ¹H NMR (400 MHz, CD₃OD) δ 8.16- 8.10 (m, 1H), 8.10- 8.03 (m, 1H), 8.01- 7.92 (m, 1H), 7.69- 7.57 (m, 1H), 7.45- 7.28 (m, 7H), 7.24- 7.08 (m, 3H), 6.18- 5.98 (m, 1H), 5.07- 4.97 (m, 1H), 4.72- 4.63 (m, 1H), 4.57- 4.36 (m, 2H), 4.10- 3.66 (m, 6H), 3.64- 3.46 (m, 2H), 2.39- 2.14 (m, 2H), 2.11- 1.77 (m, 8H), 1.72- 1.46 (m, 4H), 1.20 (d, J = 7.3 Hz, 3H), 0.93-0.79 (m, 3H) |
| | yl)fluoromethyl) (phenoxy)phosphoryl)- L-alaninate | 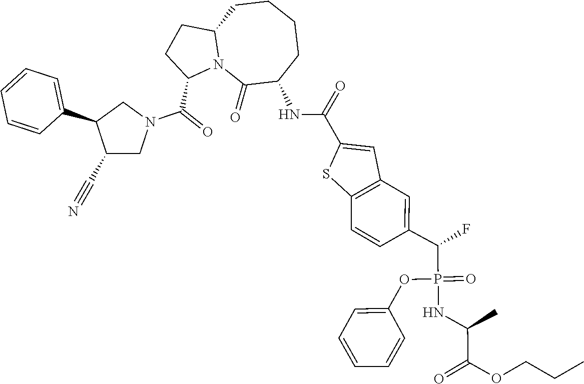 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6ccccc6)[C@@H](C#N)C5)N4C3=O)cc2c1] Pyrrolidine building block SFC peak 2 was used for synthesis and biological testing | | |
| 344 | propyl (((R)-(2- (((3S,6S,10aS)-3- ((6S,7R)-6-(3- chlorophenyl)-7- cyano-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6R,7S)-6-(3- chlorophenyl)-7- cyano-4- azaspiro[2.4]heptane- 4-carbonyl)-5- oxodecahydropyrrolo | 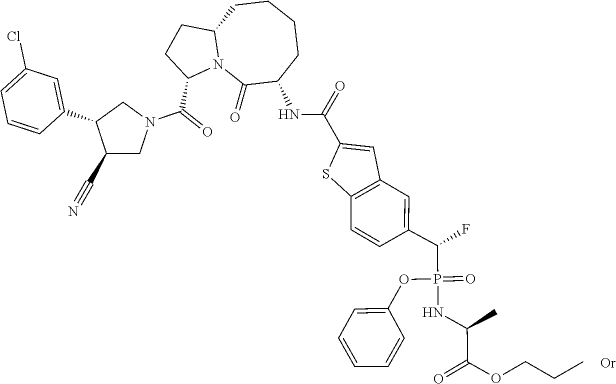 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](C#N)[C@H](c6cccc(Cl)c6)C5)N4C3=O)cc2c1] | 902.1 | ¹H NMR (400 MHz, CD₃OD) δ 8.14- 8.06 (m, 2H), 7.97- 7.94 (m, 1H), 7.63 (t, J = 8.9 Hz, 1H), 7.47 (s, 1H), 7.39-7.29 (m, 5H), 7.24-7.12 (m, 3H), 6.19-5.97 (m, 1H), 5.06-4.98 (m, 1H), 4.67-4.54 (m, 2H), 4.43-4.34 (m, 1H), 3.98 (t, J = 6.6 Hz, 1H), 3.93- 3.79 (m, 3H), 3.77- 3.73 (m, 1H), 2.36- 2.14 (m, 3H), 2.06- 1.88 (m, 7H), 1.85- 1.75 (m, 2H), 1.69- 1.46 (m, 4H), 1.29- 1.27 (m, 1H), 1.21 (d, J = 7.1 Hz, 3H), 1.03-0.94 (m, 1H), 0.90-0.83 (m, 3H), |
| | [1,2-a]azocin-6- yl)carbamoyl)benzo [b]thiophen-5- yl)fluoromethyl) (phenoxy)phosphoryl)- L-alaninate | 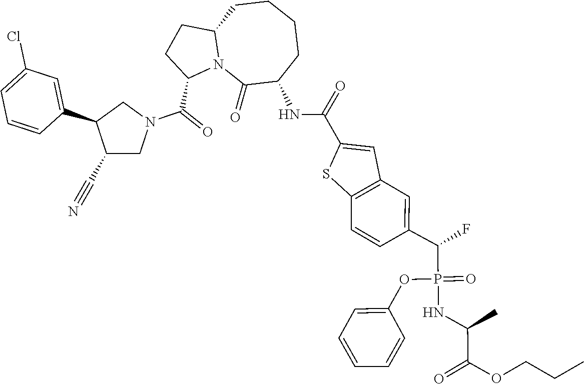 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccc(Cl)c6)[C@@H](C#N)C5)N4C3=O)cc2c1] Pyrrolidine + Core SFC peak 1 was used for synthesis and biological testing | | 0.78-0.69 (m, 1H) |
| 345 | propyl (((R)-(2- (((3S,6S,10aS)-3- ((6S,7R)-6-(3- chlorophenyl)-7- cyano-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6R,7S)-6-(3- chlorophenyl)-7- cyano-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 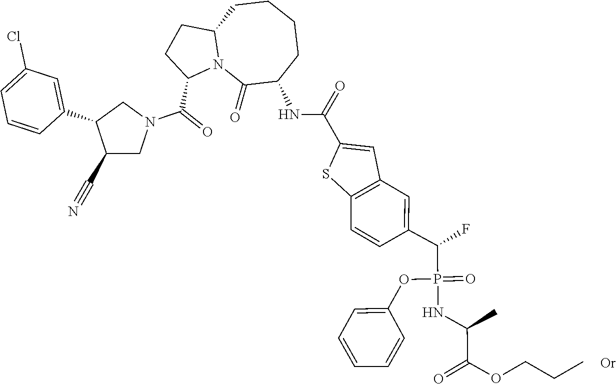 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](C#N)[C@H](c6cccc(Cl)c6)C5)N4C3=O)cc2c1] 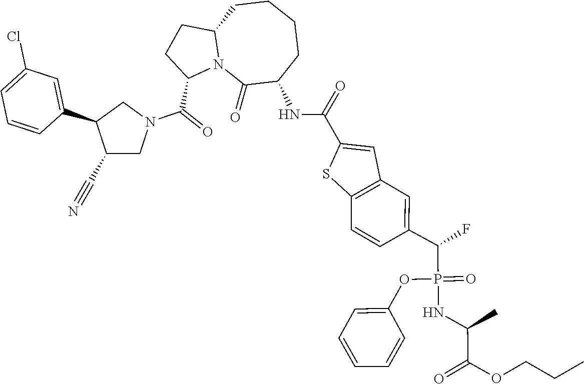 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccc(Cl)c6)[C@@H](C#N)C5)N4C3=O)cc2c1] Pyrrolidine + Core SFC peak 2 was used for synthesis and biological testing | 902.1 | ¹H NMR (400 MHz, CD₃OD) δ 8.14- 8.06 (m, 2H), 7.98- 7.95 (m, 1H), 7.63 (t, J = 8.7 Hz, 1H), 7.54 (s, 1H), 7.45-7.39 (m, 1H), 7.37-7.27 (m, 4H), 7.24-7.11 (m, 3H), 6.19-5.98 (m, 1H), 5.06-4.96 (m, 1H), 4.62-4.53 (m, 1H), 4.44-4.34 (m, 1H), 4.20-4.07 (m, 2H), 3.98 (t, J = 6.7 Hz, 1H), 3.94- 3.76 (m, 3H), 3.73- 3.68 (m, 1H), 2.42- 2.15 (m, 3H), 2.07- 1.89 (m, 6H), 1.86- 1.73 (m, 3H), 1.69- 1.46 (m, 4H), 1.35- 1.27 (m, 1H), 1.21 (d, J = 7.1 Hz, 3H), 1.02-0.94 (m, 1H), 0.92-0.81 (m, 3H), 0.74-0.65 (m, 1H) |
| 346 | propyl (((R)- fluoro(2- (((3S,6S,9aS)-3- ((R)-6-(1-methyl-2- oxo-1,2- dihydropyridin-4- yl)-4- azaspiro[2.4]heptane- 4-carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 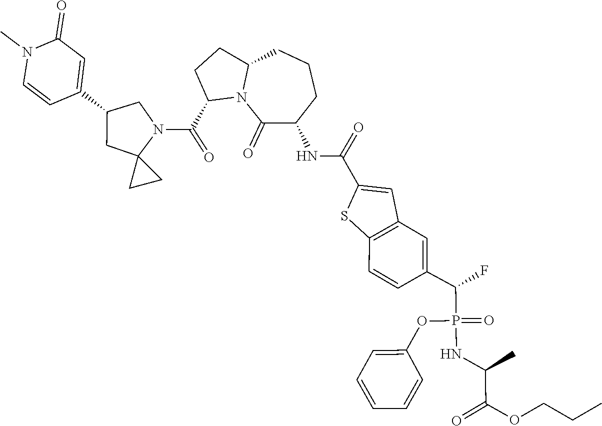 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6ccn(C)c(=O)c6)CC56CC6)N4C3=O)cc2c1] | 860.3 | ¹H NMR (400 MHz, DMSO) δ 8.84-8.75 (m, 1H), 8.27 (s, 1H), 8.13-8.02 (m, 2H), 7.67-7.56 (m, 2H), 7.40-7.30 (m, 2H), 7.24-7.08 (m, 3H), 6.33 (s, 1H), 6.29- 6.09 (m, 3H), 4.74- 4.53 (m, 2H), 4.20 (s, 1H), 3.91 (s, 3H), 3.77-3.63 (m, 1H), 3.61-3.48 (m, 1H), 3.37 (s, 3H), 2.23- 2.13 (m, 2H), 2.10- 1.91 (m, 4H), 1.89- 1.42 (m, 11H), 1.17 (d, J = 6.6 Hz, 1H), 0.94 (d, J = 6.5 Hz, 2H), 0.81 (t, J = 7.3 Hz, 3H), 0.54-0.41 (m, 2H) |
| 347 | propyl (((S)-(7- (((3S,6S,10aS)-3- ((6S,7R)-7-cyano- 6-phenyl-4- azaspiro[2.4]heptane- 4-carbonyl)-5- oxodecahydropyrrolo [1,2-a]azocin-6- yl)carbamoyl) naphthalen-2- yl)fluoromethyl) (phenoxy)phosphoryl)- L-alaninate | 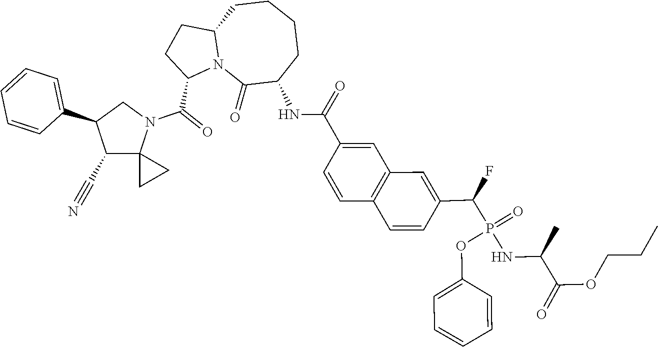 [SMILES: CCCOC(=O)[C@H](C)NP(=O)(Oc1ccccc1)[C@H](F)c1ccc2ccc(C(=O)N[C@H]3CCCC[C@H]4CC[C@@H](C(=O)N5C[C@H](c6ccccc6)[C@@H](C#N)C56CC6)N4C3=O)cc2c1] | 862.4 | ¹H NMR (400 MHz, CDCl₃) δ 8.34 (s, 1H), 8.02 (s, 1H), 7.93-7.82 (m, 3H), 7.74-7.65 (m, 1H), 7.51-7.42 (m, 1H), 7.41-7.34 (m, 3H), 7.34-7.30 (m, 3H), 7.25-7.16 (m, 2H), 7.12-7.04 (m, 1H), 6.16-5.91 (m, 1H), 5.26-5.09 (m, 1H), 4.77-4.64 (m, 1H), 4.55-4.45 (m, 1H), 4.38-4.26 (m, 1H), 4.08-3.99 (m, 2H), 3.98-3.90 (m, 1H), 3.85-3.78 (m, 2H), 3.69-3.58 (m, 1H), 3.57-3.47 (m, 1H), 2.50-2.37 (m, 1H), 2.26-2.03 (m, 5H), 2.02-1.88 (m, 3H), 1.87-1.74 (m, 3H), 1.74-1.55 (m, 3H), 1.49-1.43 (m, 1H), 1.31-1.24 (m, 3H), 1.18-1.04 (m, 1H), 0.96-0.87 (m, 1H), 0.85-0.77 (m, 2H), 0.77-0.66 (m, 1H) |
| 348 | 2-methoxyethyl N- (((S)-(2- (((3S,6S,9aS)-3- ((6S,7R)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- O-methyl-L- serinate | 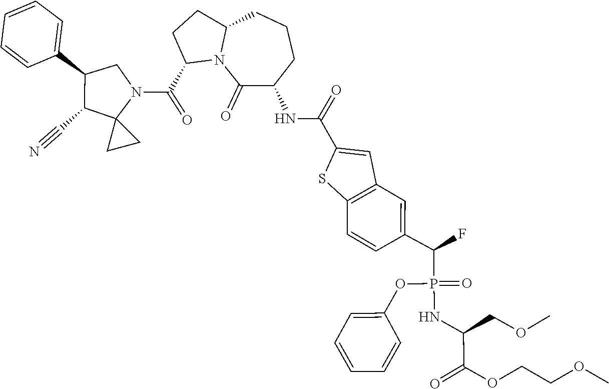 [SMILES: COCCOC(=O)[C@H](COC)NP(=O)(Oc1ccccc1)[C@H](F)c1ccc2sc(C(=O)N[C@H]3CCC[C@H]4CC[C@@H](C(=O)N5C[C@H](c6ccccc6)[C@@H](C#N)C56CC6)N4C3=O)cc2c1] | 900.2 | ¹H NMR (400 MHz, DMSO) δ 8.88-8.78 (m, 1H), 8.28 (s, 1H), 8.13-8.03 (m, 2H), 7.64-7.57 (m, 1H), 7.53-7.46 (m, 2H), 7.42-7.29 (m, 5H), 7.23-7.09 (m, 3H), 6.30-6.08 (m, 2H), 4.70-4.58 (m, 2H), 4.38-4.26 (m, 1H), 4.12-3.66 (m, 7H), 3.49-3.34 (m, 3H), 3.29-2.98 (m, 7H), 2.27-2.11 (m, 2H), 2.08-1.91 (m, 2H), 1.89-1.63 (m, 8H), 0.88-0.63 (m, 2H) |
| 349 | propyl (((S)-(2- (((3S,6S,8R,9aR)- 3-((6S,7R)-7- cyano-6-phenyl-4- azaspiro[2.4]heptane- 4-carbonyl)-8- methyl-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 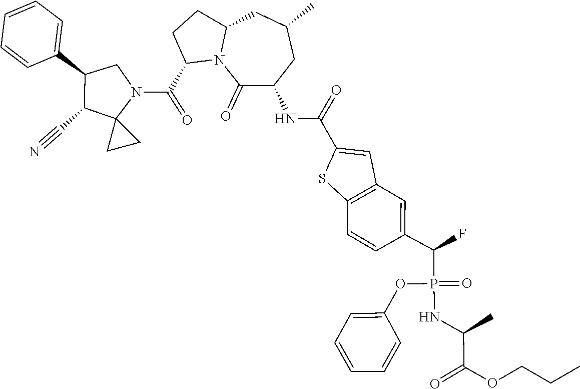 [SMILES: CCCOC(=O)[C@H](C)NP(=O)(Oc1ccccc1)[C@H](F)c1ccc2sc(C(=O)N[C@H]3C[C@@H](C)C[C@H]4CC[C@@H](C(=O)N5C[C@H](c6ccccc6)[C@@H](C#N)C56CC6)N4C3=O)cc2c1] | 868.5 | ¹H NMR (400 MHz, DMSO) δ 9.04-8.73 (m, 1H), 8.28 (s, 1H), 8.17-7.98 (m, 2H), 7.68-7.57 (m, 1H), 7.55-7.45 (m, 2H), 7.44-7.29 (m, 5H), 7.26-7.09 (m, 3H), 6.44-5.91 (m, 2H), 4.81-4.53 (m, 2H), 4.33 (s, 1H), 4.12- 3.62 (m, 7H), 2.31- 2.11 (m, 2H), 2.09- 1.87 (m, 2H), 1.87- 1.75 (m, 2H), 1.74- 1.61 (m, 3H), 1.58- 1.35 (m, 4H), 1.24- 1.10 (m, 2H), 0.99- 0.92 (m, 4H), 0.90- 0.75 (m, 4H), 0.75- 0.60 (m, 1H) |
| 350 | cyclopropyl (((S)- (2-(((3S,6S,9aS)-3- ((6S,7R)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 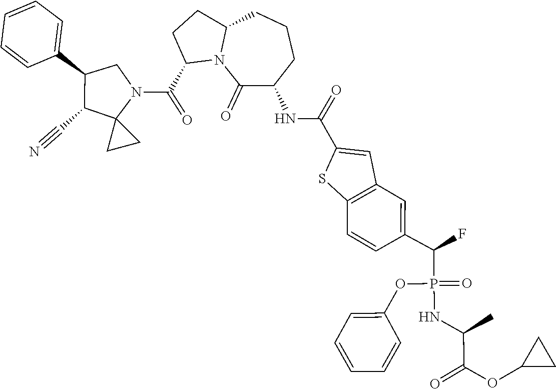 [SMILES: C[C@H](NP(=O)(Oc1ccccc1)[C@H](F)c1ccc2sc(C(=O)N[C@H]3CCC[C@H]4CC[C@@H](C(=O)N5C[C@H](c6ccccc6)[C@@H](C#N)C56CC6)N4C3=O)cc2c1)C(=O)OC1CC1] | 852.2 | ¹H NMR (400 MHz, CDCl₃) δ 7.96 (s, 1H), 7.90-7.83 (m, 1H), 7.83-7.78 (m, 1H), 7.66 (s, 1H), 7.62-7.52 (m, 1H), 7.47-7.30 (m, 6H), 7.26-7.15 (m, 2H), 7.14-7.01 (m, 2H), 6.04-5.87 (m, 1H), 5.30 (s, 1H), 4.75- 4.43 (m, 3H), 4.12- 4.05 (m, 1H), 3.91- 3.77 (m, 2H), 3.70- 3.48 (m, 3H), 2.47- 1.64 (m, 13H), 1.30- 1.01 (m, 4H), 0.78- 0.56 (m, 4H) |
| 351 | 2-methoxyethyl (((S)-(2- (((3S,6S,9aS)-3- ((6S,7R)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 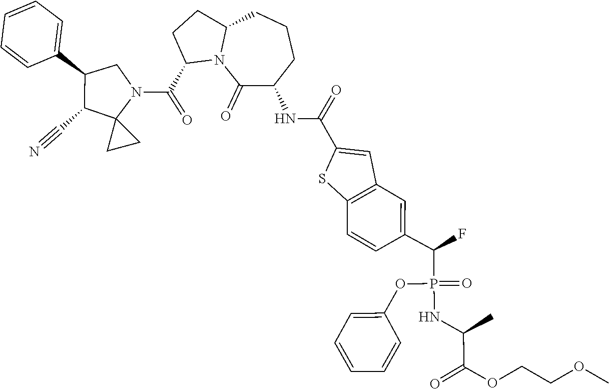 [SMILES: COCCOC(=O)[C@H](C)NP(=O)(Oc1ccccc1)[C@H](F)c1ccc2sc(C(=O)N[C@H]3CCC[C@H]4CC[C@@H](C(=O)N5C[C@H](c6ccccc6)[C@@H](C#N)C56CC6)N4C3=O)cc2c1] | 870.3 | ¹H NMR (400 MHz, CDCl₃) δ 7.98-7.90 (m, 1H), 7.89-7.83 (m, 1H), 7.82-7.78 (m, 1H), 7.73-7.51 (m, 2H), 7.46-7.29 (m, 7H), 7.25-7.23 (m, 1H), 7.21-7.07 (m, 2H), 6.05-5.87 (m, 1H), 4.68-4.53 (m, 2H), 4.37-4.02 (m, 3H), 3.95-3.42 (m, 8H), 3.37-3.29 (m, 3H), 2.45-2.35 (m, 1H), 2.29-2.12 (m, 2H), 2.01-1.64 (m, 9H), 1.33-1.25 (m, 3H), 1.18-1.09 (m, 1H), 0.83-0.69 (m, 1H) |
| 352 | propyl (((S)-(2- (((3S,6S,8S,9aR)-3- ((6S,7R)-7-cyano- 6-phenyl-4- azaspiro[2.4]heptane- 4-carbonyl)-8- methyl-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 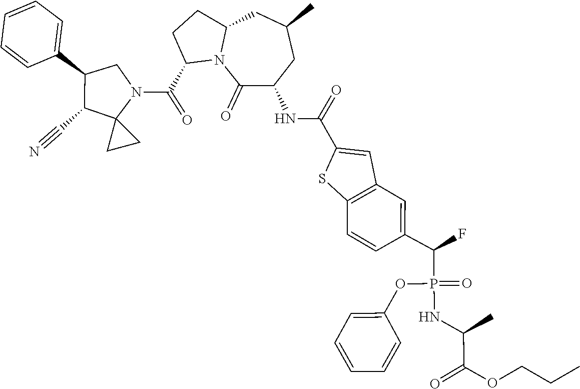 [SMILES: CCCOC(=O)[C@H](C)NP(=O)(Oc1ccccc1)[C@H](F)c1ccc2sc(C(=O)N[C@H]3C[C@H](C)C[C@H]4CC[C@@H](C(=O)N5C[C@H](c6ccccc6)[C@@H](C#N)C56CC6)N4C3=O)cc2c1] | 868.3 | ¹H NMR (400 MHz, DMSO) δ 8.90-8.73 (m, 1H), 8.34-8.19 (m, 1H), 8.14-8.00 (m, 2H), 7.69-7.54 (m, 1H), 7.50 (d, J = 7.5 Hz, 2H), 7.43- 7.29 (m, 5H), 7.25- 7.09 (m, 3H), 6.31- 6.03 (m, 2H), 4.92- 4.79 (m, 1H), 4.66- 4.55 (m, 1H), 4.39- 4.28 (m, 1H), 4.13 (d, J = 6.7 Hz, 1H), 4.02 (d, J = 9.9 Hz, 1H), 3.94-3.80 (m, 3H), 3.80-3.60 (m, 3H), 2.30-1.79 (m, 8H), 1.74-1.61 (m, 3H), 1.54-1.41 (m, 3H), 1.21-1.08 (m, 4H), 0.95 (d, J = 7.1 Hz, 2H), 0.88-0.76 (m, 4H), 0.74-0.64 (m, 1H) |
| 353 | propyl (((R)- fluoro(2- (((3S,6S,8R,9aR)- 8-methyl-5-oxo-3- ((R)-6-(pyridin-3- yl)-4- azaspiro[2.4]heptane- 4- carbonyl)octahydro- 1H-pyrrolo[1,2- a]azepin-6- yl)carbamoyl)benzo [b]thiophen-5- yl)methyl)(phenoxy) phosphoryl)-L- alaninate | 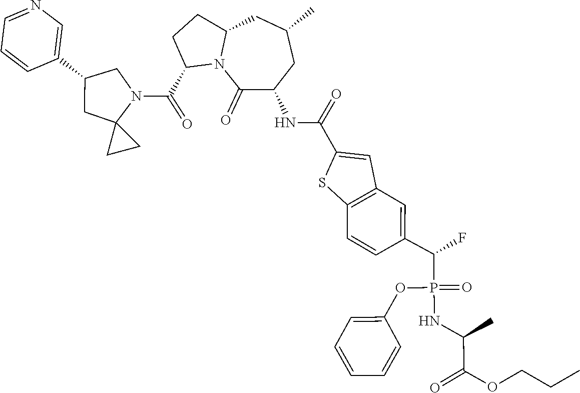 [SMILES: CCCOC(=O)[C@H](C)NP(=O)(Oc1ccccc1)[C@@H](F)c1ccc2sc(C(=O)N[C@H]3C[C@@H](C)C[C@H]4CC[C@@H](C(=O)N5C[C@@H](c6cccnc6)CC56CC6)N4C3=O)cc2c1] | 844.3 | ¹H NMR (400 MHz, CDCl₃) δ 8.86-8.77 (m, 1H), 8.58 (s, 1H), 8.43 (d, J = 4.5 Hz, 1H), 8.27 (s, 1H), 8.07 (d, J = 5.3 Hz, 2H), 7.79 (d, J = 7.9 Hz, 1H), 7.61 (d, J = 8.2 Hz, 1H), 7.37- 7.30 (m, 3H), 7.19- 7.13 (m, 3H), 6.28- 6.19 (m, 1H), 6.16- 6.08 (m, 1H), 4.67- 4.58 (m, 2H), 4.09 (t, J = 8.5 Hz, 1H), 4.01- 3.89 (m, 2H), 3.85- 3.71 (m, 3H), 3.62- 3.53 (m, 1H), 2.23- 1.94 (m, 6H), 1.85- 1.66 (m, 4H), 1.62- 1.49 (m, 3H), 1.47- 1.37 (m, 2H), 1.13 (d, J = 6.8 Hz, 3H), 0.96 (d, J = 6.4 Hz, 3H), 0.84-0.76 (m, 3H), 0.52-0.41 (m, 2H) |
| 354 | propyl (((S)-(2- (((3S,6S,9aS)-3- ((6S,7R)-7-cyano- 6-(pyridin-3-yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((S)-(2- (((3S,6S,9aS)-3- ((6R,7S)-7-cyano- 6-(pyridin-3-yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 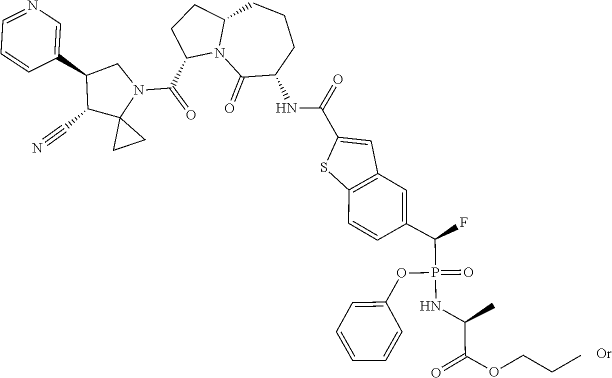 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5C[C@H](c6cccnc6)[C@@H](C#N)C56CC6)N4C3=O)cc2c1] 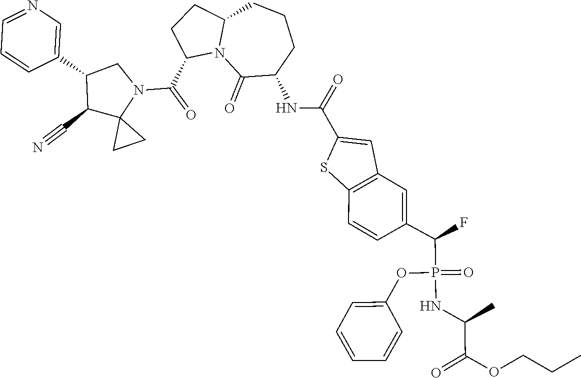 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5C[C@@H](c6cccnc6)[C@H](C#N)C56CC6)N4C3=O)cc2c1] Pyrrolidine + Core SFC peak 2 was used for synthesis and biological testing | 855.5 | ¹H NMR (400 MHz, DMSO) δ 8.92-8.77 (m, 1H), 8.71 (s, 1H), 8.60-8.45 (m, 1H), 8.27 (d, J = 3.7 Hz, 1H), 8.17-8.01 (m, 2H), 7.99-7.88 (m, 1H), 7.64-7.51 (m, 1H), 7.46-7.41 (m, 1H), 7.40-7.31 (m, 2H), 7.24-7.10 (m, 3H), 6.34-6.03 (m, 2H), 4.72-4.55 (m, 2H), 4.35 (t, J = 8.4 Hz, 1H), 4.07 (d, J = 9.8 Hz, 1H), 3.99- 3.65 (m, 6H), 2.25- 2.12 (m, 2H), 2.09- 1.91 (m, 2H), 1.88- 1.63 (m, 8H), 1.56- 1.41 (m, 2H), 1.19- 0.92 (m, 3H), 0.87- 0.76 (m, 4H), 0.75- 0.64 (m, 1H) |
| 355 | propyl (((S)-(2- (((3S,6S,9aS)-3- ((6S,7R)-7-cyano- 6-(pyridin-3-yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((S)-(2- (((3S,6S,9aS)-3- ((6R,7S)-7-cyano- 6-(pyridin-3-yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 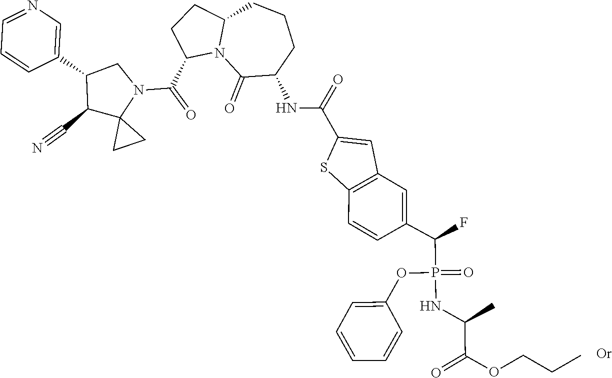 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5C[C@@H](c6cccnc6)[C@H](C#N)C56CC6)N4C3=O)cc2c1] 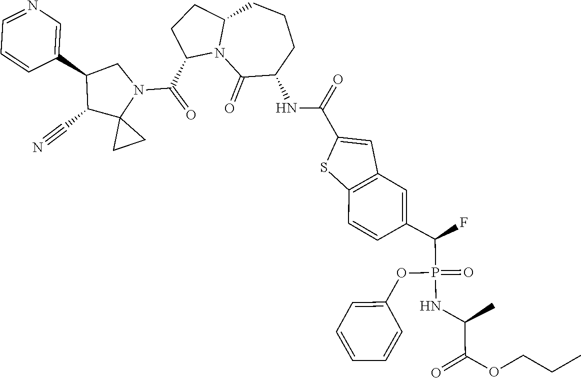 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5C[C@H](c6cccnc6)[C@@H](C#N)C56CC6)N4C3=O)cc2c1] Pyrrolidine + Core SFC peak 1 was used for synthesis and biological testing | 855.3 | ¹H NMR (400 MHz, CDCl₃) δ 8.70 (s, 1H), 8.67-8.61 (m, 1H), 7.97 (s, 1H), 7.91-7.79 (m, 3H), 7.67-7.54 (m, 2H), 7.50-7.32 (m, 2H), 7.26-7.17 (m, 2H), 7.14-7.06 (m, 2H), 5.95 (dd, J = 44.0, 5.3 Hz, 1H), 4.74-4.60 (m, 2H), 4.26-3.74 (m, 7H), 3.68-3.58 (m, 1H), 3.35-3.27 (m, 1H), 2.50-2.39 (m, 1H), 2.32-2.22 (m, 1H), 2.08-1.70 (m, 12H), 1.34-1.25 (m, 3H), 1.08-0.98 (m, 1H), 0.95-0.82 (m, 3H), 0.78-0.66 (m, 1H) |
| 356 | (1-methyl-1H- tetrazol-5-yl)methyl (((R)-fluoro(2- (((3S,6S,9aS)-5- oxo-3-((R)-6- (pyridin-3-yl)-4- azaspiro[2.4]heptane- 4- carbonyl)octahydro- 1H-pyrrolo[1,2- a]azepin-6- yl)carbamoyl)benzo [b]thiophen-5- yl)methyl)(phenoxy) phosphoryl)-L- alaninate | 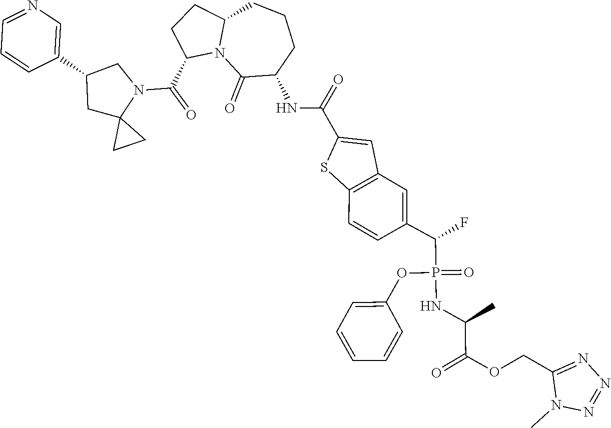 [SMILES: C[C@H](NP(=O)(Oc1ccccc1)[C@@H](F)c1ccc2sc(C(=O)N[C@H]3CCC[C@H]4CC[C@@H](C(=O)N5C[C@@H](c6cccnc6)CC56CC6)N4C3=O)cc2c1)C(=O)OCc1nnnn1C] | 884.1 | ¹H NMR (400 MHz, DMSO) δ 8.80 (d, J = 7.5 Hz, 1H), 8.59 (s, 1H), 8.44 (d, J = 4.6 Hz, 1H), 8.38-8.25 (m, 2H), 8.11-8.04 (m, 2H), 7.80 (d, J = 7.9 Hz, 1H), 7.59 (d, J = 8.2 Hz, 1H), 7.37- 7.30 (m, 3H), 7.16- 7.11 (m, 2H), 6.29- 6.13 (m, 2H), 5.41- 5.34 (m, 2H), 4.67- 4.58 (m, 2H), 4.18- 4.06 (m, 2H), 4.04 (s, 3H), 4.00-3.93 (m, 1H), 3.73 (t, J = 9.5 Hz, 1H), 3.60-3.56 (m, 1H), 2.24-1.98 (m, 6H), 1.82-1.68 (m, 6H), 1.23-1.18 (m, 3H), 1.14-1.08 (m, 2H), 0.54-0.42 (m, 2H) |
| 357 | propyl (((R)- fluoro(2- (((3S,6S,9aS)-5- oxo-3-((R)-6- (pyridin-3-yl)-4- azaspiro[2.4]heptane- 4- carbonyl)octahydro- 1H-pyrrolo[1,2- a]azepin-6- yl)carbamoyl)benzo [b]thiophen-5- yl)methyl)(3-(2- methoxyethoxy) phenoxy)phosphoryl)- L-alaninate | 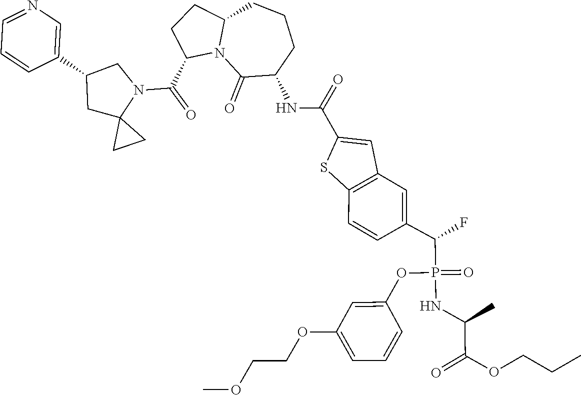 [SMILES: CCCOC(=O)[C@H](C)NP(=O)(Oc1cccc(OCCOC)c1)[C@@H](F)c1ccc2sc(C(=O)N[C@H]3CCC[C@H]4CC[C@@H](C(=O)N5C[C@@H](c6cccnc6)CC56CC6)N4C3=O)cc2c1] | 904.3 | ¹H NMR (400 MHz, CDCl₃) δ 8.78-8.43 (m, 2H), 8.01-7.92 (m, 1H), 7.91-7.84 (m, 2H), 7.84-7.75 (m, 2H), 7.62-7.50 (m, 1H), 7.43-7.33 (m, 1H), 7.25-7.09 (m, 1H), 6.90-6.75 (m, 1H), 6.71-6.59 (m, 2H), 6.04-5.85 (m, 1H), 4.75-4.61 (m, 2H), 4.14-3.85 (m, 8H), 3.77-3.67 (m, 2H), 3.66-3.50 (m, 2H), 3.46-3.39 (m, 3H), 2.34-2.13 (m, 5H), 2.11-1.88 (m, 6H), 1.86-1.71 (m, 3H), 1.67-1.55 (m, 2H), 1.30 (d, J = 7.1 Hz, 2H), 1.12 (d, J = 7.1 Hz, 1H), 0.94- 0.87 (m, 3H), 0.57- 0.46 (m, 2H) |
| 358 | 2-methoxy-2- methylpropyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 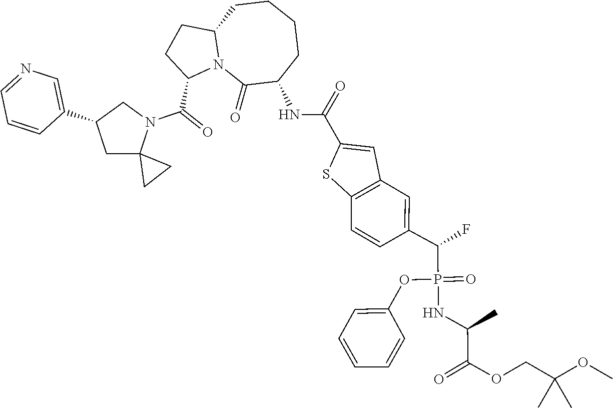 [SMILES: COC(C)(C)COC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | 888.4 | ¹H NMR (400 MHz, CDCl₃) δ 9.56 (s, 1H), 8.78-8.65 (m, 1H), 8.34-8.12 (m, 2H), 8.10-7.89 (m, 2H), 7.85-7.70 (m, 2H), 7.60-7.45 (m, 1H), 7.38-7.27 (m, 1H), 7.26-7.14 (m, 2H), 7.14-7.03 (m, 2H), 6.08-5.84 (m, 1H), 5.24-5.14 (m, 1H), 4.81-4.71 (m, 1H), 4.56-4.49 (m, 1H), 4.35-4.29 (m, 1H), 4.08-3.99 (m, 3H), 3.68-3.63 (m, 1H), 3.25-3.12 (m, 3H), 2.77-2.66 (m, 1H), 2.18-1.95 (m, 9H), 1.86-1.68 (m, 4H), 1.67-1.56 (m, 2H), 1.33-1.25 (m, 3H), 1.22-1.08 (m, 7H), 0.48-0.39 (m, 1H), 0.21-0.10 (m, 1H) |
| 359 | propyl ((2,6- dimethylphenoxy) ((S)-fluoro(2- (((3S,6S,9aS)-5- oxo-3-((R)-6- (pyridin-3-yl)-4- azaspiro[2.4]heptane- 4- carbonyl)octahydro- 1H-pyrrolo[1,2- a]azepin-6- yl)carbamoyl)benzo [b]thiophen-5- yl)methyl)phosphoryl)- L-alaninate | 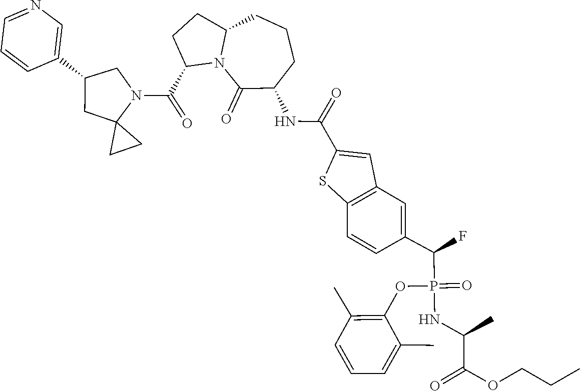 [SMILES: CCCOC(=O)[C@H](C)NP(=O)(Oc1c(C)cccc1C)[C@H](F)c1ccc2sc(C(=O)N[C@H]3CCC[C@H]4CC[C@@H](C(=O)N5C[C@@H](c6cccnc6)CC56CC6)N4C3=O)cc2c1] | 858.9 | ¹H NMR (400 MHz, CDCl₃) δ 8.69-8.51 (m, 2H), 8.03-7.98 (m, 1H), 7.91-7.74 (m, 4H), 7.66-7.60 (m, 1H), 7.41-7.34 (m, 1H), 7.03-6.91 (m, 3H), 6.05-5.87 (m, 1H), 4.76-4.59 (m, 2H), 4.17-3.86 (m, 6H), 3.65-3.55 (m, 2H), 2.38-2.23 (m, 4H), 2.22-2.18 (m, 6H), 2.17-2.10 (m, 2H), 2.03-1.94 (m, 5H), 1.83-1.72 (m, 3H), 1.63-1.52 (m, 2H), 1.35-1.23 (m, 3H), 0.90-0.81 (m, 3H), 0.58-0.46 (m, 2H). |
| 360 | (tetrahydro-2H- pyran-4-yl)methyl (((R)-fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 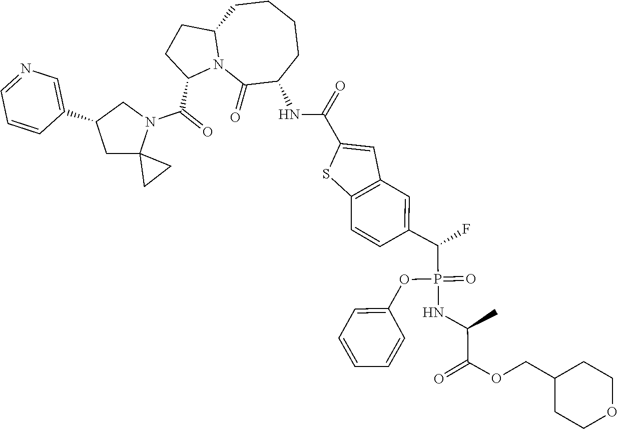 [SMILES: C[C@H](NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1)C(=O)OCC1CCOCC1] | 900.4 | ¹H NMR (400 MHz, CDCl₃) δ 9.54-9.36 (m, 1H), 8.79-8.67 (m, 1H), 8.28-8.22 (m, 1H), 8.05-7.96 (m, 2H), 7.94-7.87 (m, 1H), 7.85-7.78 (m, 1H), 7.76-7.67 (m, 1H), 7.61-7.47 (m, 1H), 7.37-7.27 (m, 1H), 7.26-7.16 (m, 2H), 7.13-7.05 (m, 2H), 6.04-5.85 (m, 1H), 5.23-5.10 (m, 1H), 4.63 (d, J = 10.5 Hz, 1H), 4.57- 4.48 (m, 1H), 4.38- 4.28 (m, 1H), 4.14- 3.99 (m, 2H), 3.98- 3.90 (m, 3H), 3.91- 3.58 (m, 3H), 3.42- 3.26 (m, 2H), 2.69- 2.62 (m, 1H), 2.25- 1.86 (m, 10H), 1.86- 1.74 (m, 4H), 1.67- 1.46 (m, 4H), 1.36- 1.24 (m, 4H), 1.15 (d, J = 7.1 Hz, 1H), 0.51-0.40 (m, 1H), 0.26-0.16 (m, 1H) |
| 361 | 2-methoxyethyl (((R)-(2- (((3S,6S,9aS)-3- ((6R,7S)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 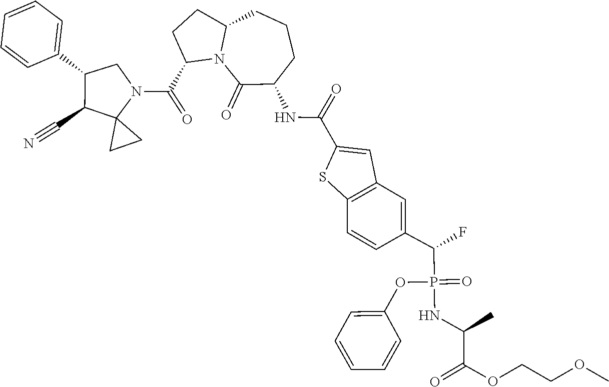 [SMILES: COCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6ccccc6)[C@H](C#N)C56CC6)N4C3=O)cc2c1] | 870.3 | ¹H NMR (400 MHz, DMSO) δ 8.84-8.76 (m, 1H), 8.27 (s, 1H), 8.10-8.04 (m, 2H), 7.64-7.58 (m, 1H), 7.52-7.46 (m, 2H), 7.40-7.29 (m, 5H), 7.20-7.11 (m, 3H), 6.30-6.12 (m, 2H), 4.66-4.57 (m, 2H), 4.19-3.87 (m, 6H), 3.83-3.76 (m, 2H), 3.48-3.39 (m, 2H), 3.23-3.19 (m, 3H), 2.24-2.05 (m, 3H), 1.99-1.92 (m, 1H), 1.86-1.66 (m, 8H), 1.18-1.12 (m, 3H), 0.86-0.77 (m, 1H), 0.70-0.61 (m, 1H) |
| 362 | propyl (((R)-(2- (((3S,6S,9aS)-3- ((6S,7R)-7-cyano- 6-(pyridin-3-yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-7-cyano- 6-(pyridin-3-yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 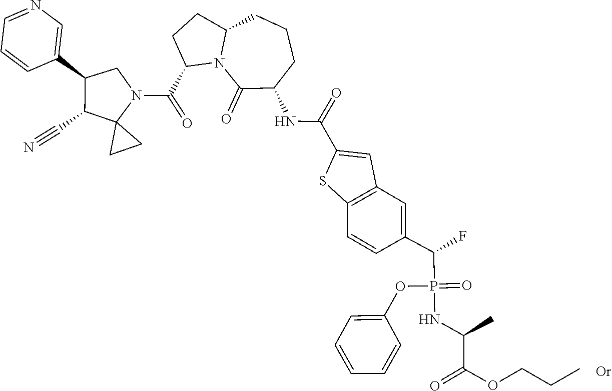 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6cccnc6)[C@@H](C#N)C56CC6)N4C3=O)cc2c1] 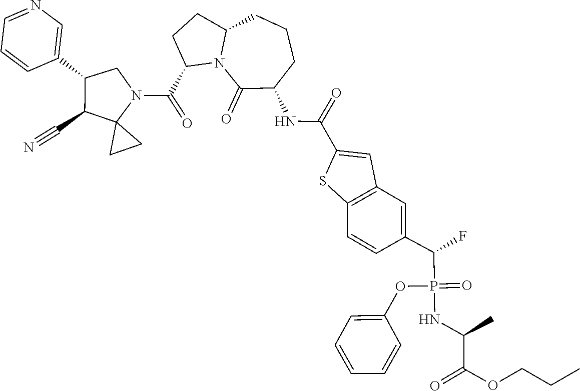 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cccnc6)[C@H](C#N)C56CC6)N4C3=O)cc2c1] Pyrrolidine + Core SFC peak 2 was used for synthesis and biological testing | 855.5 | ¹H NMR (400 MHz, DMSO) δ 8.95-8.77 (m, 2H), 8.69 (s, 1H), 8.28 (s, 1H), 8.23 (d, J = 7.7 Hz, 1H), 8.08 (d, J = 4.6 Hz, 2H), 7.75-7.57 (m, 2H), 7.36 (t, J = 7.9 Hz, 2H), 7.18 (t, J = 9.4 Hz, 3H), 6.31-6.04 (m, 2H), 4.72-4.53 (m, 2H), 4.38 (t, J = 8.7 Hz, 1H), 4.10 (d, J = 9.9 Hz, 1H), 4.03- 3.89 (m, 3H), 3.88- 3.64 (m, 3H), 2.27- 2.12 (m, 2H), 2.09- 1.91 (m, 2H), 1.89- 1.61 (m, 8H), 1.58- 1.34 (m, 2H), 1.21- 1.03 (m, 3H), 0.92- 0.75 (m, 4H), 0.74- 0.64 (m, 1H) |
| 363 | propyl (((R)-(2- (((3S,6S,9aS)-3- ((6S,7R)-7-cyano- 6-(pyridin-3-yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-7-cyano- 6-(pyridin-3-yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 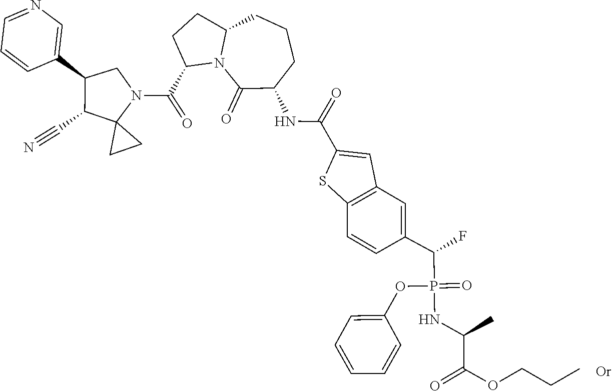 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6cccnc6)[C@@H](C#N)C56CC6)N4C3=O)cc2c1] 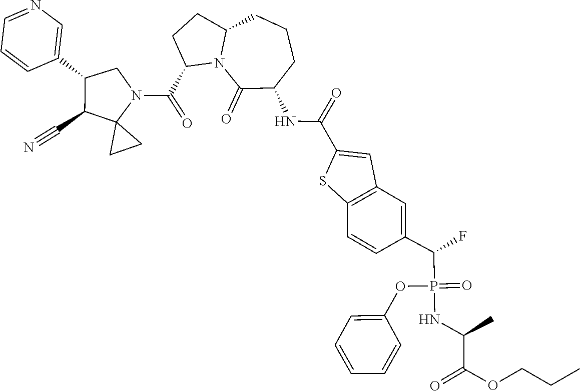 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cccnc6)[C@H](C#N)C56CC6)N4C3=O)cc2c1] Pyrrolidine + Core SFC peak 1 was used for synthesis and biological testing | 855.5 | ¹H NMR (400 MHz, DMSO) δ 8.88-8.77 (m, 1H), 8.74 (s, 1H), 8.55 (d, J = 4.3 Hz, 1H), 8.27 (s, 1H), 8.19-7.97 (m, 3H), 7.61 (d, J = 8.0 Hz, 1H), 7.47 (dd, J = 7.6, 5.0 Hz, 1H), 7.36 (t, J = 7.8 Hz, 2H), 7.25-7.11 (m, 3H), 6.31-6.02 (m, 2H), 4.71-4.50 (m, 2H), 4.27-4.09 (m, 1H), 4.04-3.89 (m, 5H), 3.85-3.73 (m, 2H), 2.26-1.92 (m, 4H), 1.90-1.65 (m, 8H), 1.58-1.38 (m, 2H), 1.19-1.07 (m, 3H), 0.90-0.72 (m, 4H), 0.70-0.53 (m, 1H) |
| 364 | propyl (((R)-(2- (((3S,6S,10aS)-3- ((6S,7R)-7-cyano- 6-(pyridin-3-yl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6R,7S)-7-cyano- 6-(pyridin-3-yl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 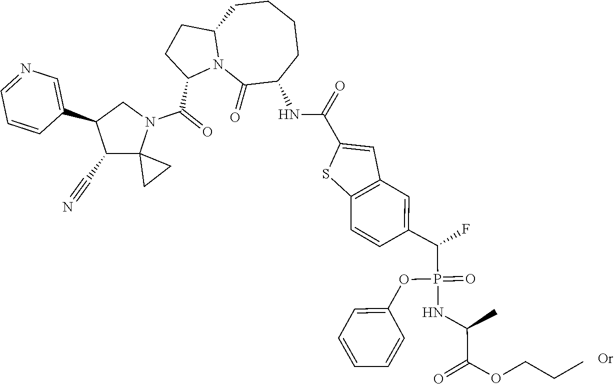 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccnc6)[C@@H](C#N)C56CC6)N4C3=O)cc2c1] 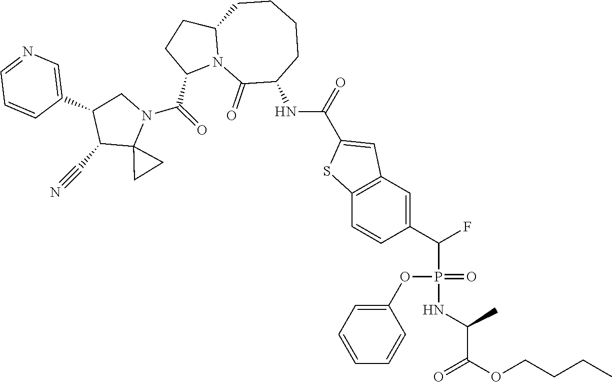 [SMILES: CCCCOC(=O)[C@H](C)NP(=O)(Oc1ccccc1)C(F)c1ccc2sc(C(=O)N[C@H]3CCCC[C@H]4CC[C@@H](C(=O)N5C[C@@H](c6cccnc6)[C@@H](C#N)C56CC6)N4C3=O)cc2c1] Pyrrolidine + Core SFC peak 2 was used for synthesis and biological testing | 869.3 | ¹H NMR (400 MHz, DMSO) δ 8.96-8.82 (m, 1H), 8.70 (s, 1H), 8.59-8.48 (m, 1H), 8.30 (s, 1H), 8.11- 8.03 (m, 2H), 7.96- 7.87 (m, 1H), 7.65- 7.56 (m, 1H), 7.46- 7.32 (m, 3H), 7.21- 7.13 (m, 3H), 6.27- 6.07 (m, 2H), 4.95 (s, 1H), 4.59-4.40 (m, 2H), 4.28-4.18 (m, 1H), 4.11-4.02 (m, 1H), 3.98-3.89 (m, 1H), 3.84-3.72 (m, 4H), 2.28-2.16 (m, 2H), 2.00-1.46 (m, 14H), 1.13 (d, J = 6.4 Hz, 3H), 0.84-0.67 (m, 5H) |
| 365 | propyl (((R)-(2- (((3S,6S,10aS)-3- ((6S,7R)-7-cyano- 6-(pyridin-3-yl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6R,7S)-7-cyano- 6-(pyridin-3-yl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 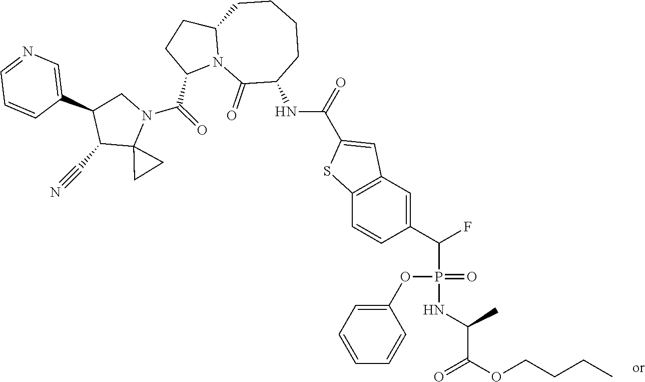 [SMILES: CCCCOC(=O)[C@H](C)NP(=O)(Oc1ccccc1)C(F)c1ccc2sc(C(=O)N[C@H]3CCCC[C@H]4CC[C@@H](C(=O)N5C[C@H](c6cccnc6)[C@@H](C#N)C56CC6)N4C3=O)cc2c1] 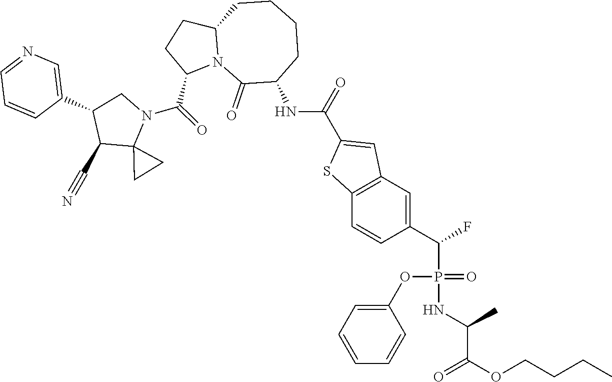 [SMILES: CCCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cccnc6)[C@H](C#N)C56CC6)N4C3=O)cc2c1] Pyrrolidine + Core SFC peak 1 was used for synthesis and biological testing | 869 | ¹H NMR (400 MHz, DMSO) δ 8.95-8.73 (m, 2H), 8.68-8.59 (m, 1H), 8.33-8.25 (m, 1H), 8.23-8.14 (m, 1H), 8.10-8.03 (m, 2H), 7.68-7.56 (m, 2H), 7.39-7.31 (m, 2H), 7.21-7.10 (m, 3H), 6.29-6.19 (m, 1H), 6.17-6.09 (m, 1H), 4.98-4.90 (m, 1H), 4.55-4.47 (m, 1H), 4.32-4.18 (m, 2H), 4.10-3.86 (m, 4H), 3.85-3.75 (m, 2H), 2.35-2.24 (m, 1H), 2.07-1.37 (m, 15H), 1.13 (d, J = 6.1 Hz, 3H), 0.87- 0.75 (m, 4H), 0.69- 0.55 (m, 1H) |
| 366 | pyridazin-3- ylmethyl (((R)- fluoro(2- (((3S,6S,9aS)-5- oxo-3-((R)-6- (pyridin-3-yl)-4- azaspiro[2.4]heptane- 4- carbonyl)octahydro- 1H-pyrrolo[1,2- a]azepin-6- yl)carbamoyl)benzo [b]thiophen-5- yl)methyl)(phenoxy) phosphoryl)-L- alaninate | 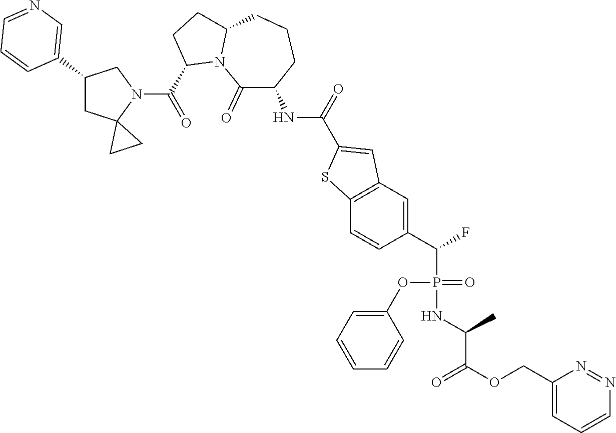 [SMILES: C[C@H](NP(=O)(Oc1ccccc1)[C@@H](F)c1ccc2sc(C(=O)N[C@H]3CCC[C@H]4CC[C@@H](C(=O)N5C[C@@H](c6cccnc6)CC56CC6)N4C3=O)cc2c1)C(=O)OCc1cccnn1] | 880.4 | ¹H NMR (400 MHz, DMSO) δ 9.20-9.14 (m, 1H), 8.83-8.74 (m, 1H), 8.61-8.55 (m, 1H), 8.46-8.41 (m, 1H), 8.28-8.23 (m, 1H), 8.09-8.02 (m, 2H), 7.84-7.78 (m, 1H), 7.71-7.55 (m, 3H), 7.37-7.25 (m, 3H), 7.19-7.10 (m, 3H), 6.41-6.14 (m, 2H), 5.36-5.20 (m, 2H), 4.67-4.58 (m, 2H), 4.14-3.84 (m, 3H), 3.77-3.69 (m, 1H), 3.63-3.52 (m, 1H), 2.24-1.94 (m, 6H), 1.88-1.67 (m, 7H), 1.63-1.55 (m, 1H), 1.25-1.16 (m, 3H), 0.54-0.40 (m, 2H) |
| 367 | propyl (((R)-(2- (((3S,6S,9aS)-3- ((6S,7R)-6-(5- chloropyridin-3-yl)- 7-cyano-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-6-(5- chloropyridin-3-yl)- 7-cyano-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 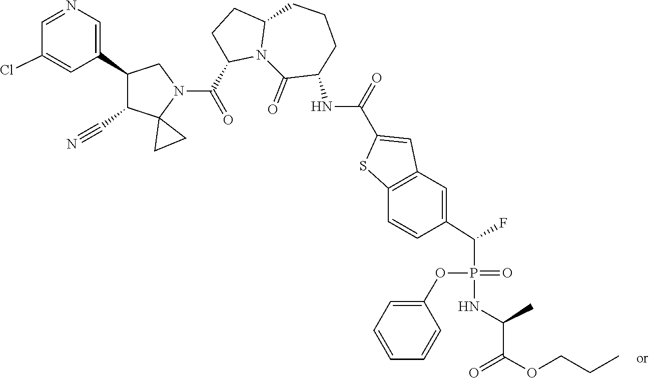 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6cncc(Cl)c6)[C@@H](C#N)C56CC6)N4C3=O)cc2c1] 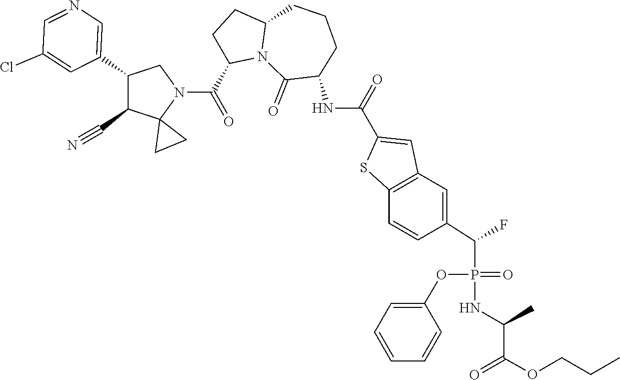 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cncc(Cl)c6)[C@H](C#N)C56CC6)N4C3=O)cc2c1] Pyrrolidine + Core SFC peak 2 was used for synthesis and biological testing | 889 | ¹H NMR (400 MHz, CDCl₃) δ 8.59 (s, 1H), 8.54 (s, 1H), 7.97 (d, J = 16.0 Hz, 1H), 7.88-7.85 (m, 1H), 7.82 (s, 1H), 7.69-7.50 (m, 3H), 7.35 (t, J = 7.6 Hz, 1H), 7.27-7.05 (m, 4H), 6.05-5.87 (m, 1H), 4.65 (s, 2H), 4.22-3.55 (m, 9H), 2.46-2.35 (m, 1H), 2.24-2.15 (m, 2H), 2.06-1.89 (m, 7H), 1.84-1.76 (m, 2H), 1.64-1.57 (m, 2H), 1.31-1.28 (m, 2H), 1.19-1.08 (m, 2H), 0.93-0.74 (m, 4H) |
| 368 | propyl (((R)-(2- (((3S,6S,9aS)-3- ((6S,7R)-6-(5- chloropyridin-3-yl)- 7-cyano-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-6-(5- chloropyridin-3-yl)- 7-cyano-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 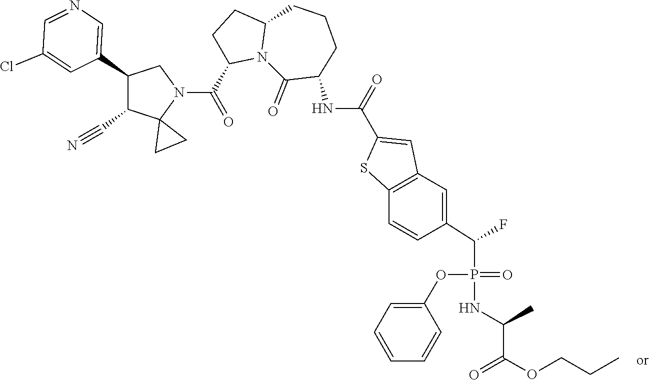 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6cncc(Cl)c6)[C@@H](C#N)C56CC6)N4C3=O)cc2c1] 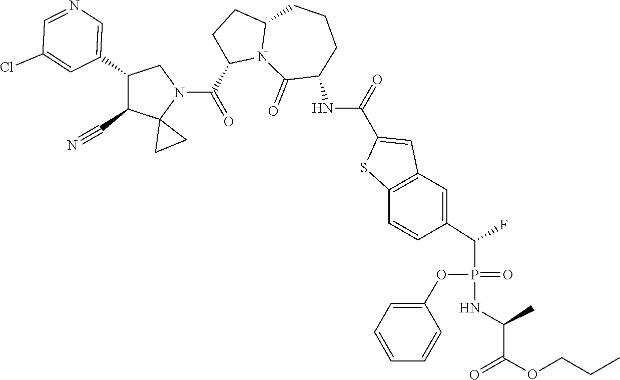 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cncc(Cl)c6)[C@H](C#N)C56CC6)N4C3=O)cc2c1] Pyrrolidine + Core SFC peak 1 was used for synthesis and biological testing | 889.2 | ¹H NMR (400 MHz, DMSO) δ 8.87-8.78 (m, 1H), 8.67 (s, 1H), 8.56 (s, 1H), 8.26 (s, 1H), 8.19 (s, 1H), 8.14-8.00 (m, 2H), 7.67-7.57 (m, 1H), 7.42-7.28 (m, 2H), 7.27-7.07 (m, 3H), 6.32-6.05 (m, 2H), 4.71-4.56 (m, 2H), 4.21-4.12 (m, 1H), 4.06-3.89 (m, 5H), 3.86-3.74 (m, 2H), 2.26-1.89 (m, 5H), 1.87-1.67 (m, 7H), 1.54-1.37 (m, 2H), 1.14 (d, J = 5.6 Hz, 3H), 0.91-0.52 (m, 5H) |
| 369 | propyl (((R)-(2- (((3S,6S,9aS)-3- ((6S,7R)-7-cyano- 6-(5- methoxypyridin-3- yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-7-cyano- 6-(5- methoxypyridin-3- yl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 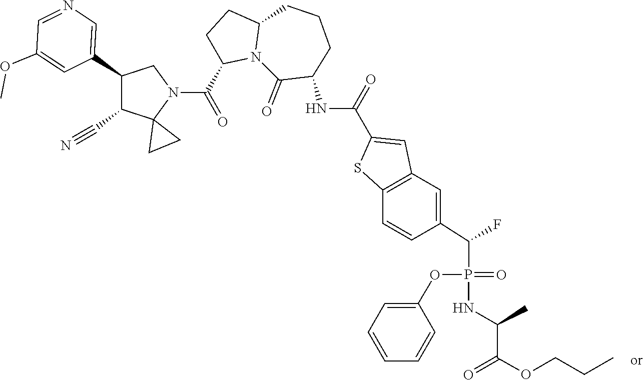 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6cncc(OC)c6)[C@@H](C#N)C56CC6)N4C3=O)cc2c1] 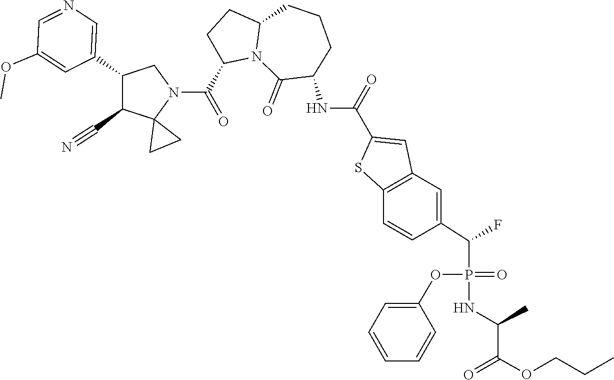 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cncc(OC)c6)[C@H](C#N)C56CC6)N4C3=O)cc2c1] Pyrrolidine + Core SFC peak 2 was used for synthesis and biological testing | | ¹H NMR (400 MHz, DMSO) δ 8.84 (d, J = 7.6 Hz, 1H), 8.34 (d, J = 10.2 Hz, 2H), 8.09-8.05 (m, 2H), 7.73-7.68 (m, 1H), 7.62 (d, J = 8.6 Hz, 1H), 7.38-7.33 (m, 2H), 7.19-7.14 (m, 3H), 6.26-6.08 (m, 2H), 4.67-4.61 (m, 2H), 4.39-4.32 (m, 1H), 4.14 (d, J = 10.1 Hz, 1H), 3.98-3.94 (m, 1H), 3.88-3.78 (m, 8H), 2.23-2.14 (m, 2H), 2.08-1.94 (m, 2H), 1.89-1.64 (m, 9H), 1.46-1.37 (m, 2H), 1.13 (d, J = 7.1 Hz, 3H), 0.85- 0.82 (m, 1H), 0.81- 0.76 (m, 3H), 0.74- 0.68 (m, 1H) |
| 370 | propyl (((R)-(2- (((3S,6S,9aS)-3- ((6S,7R)-6-(5- chloropyridin-3-yl)- 7-cyano-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-6-(5- chloropyridin-3-yl)- 7-cyano-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 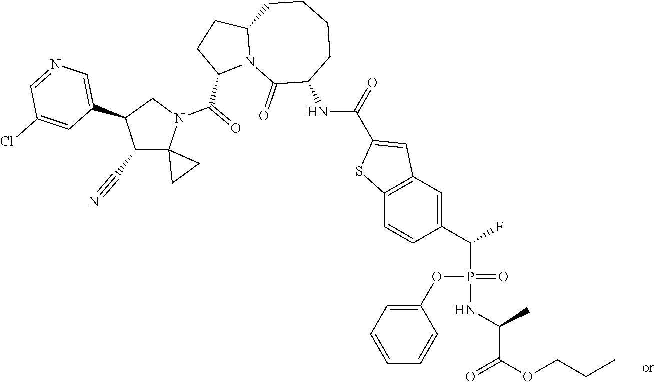 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cncc(Cl)c6)[C@@H](C#N)C56CC6)N4C3=O)cc2c1] 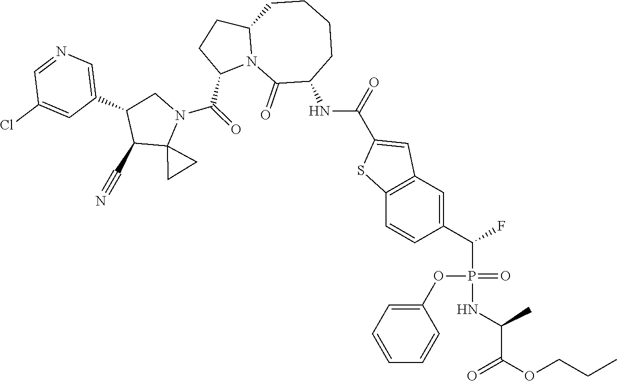 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cncc(Cl)c6)[C@H](C#N)C56CC6)N4C3=O)cc2c1] Pyrrolidine + Core SFC peak 2 was used for synthesis and biological testing | 903.2 | ¹H NMR (400 MHz, DMSO) δ 8.96-8.84 (m, 1H), 8.67 (d, J = 1.3 Hz, 1H), 8.60 (d, J = 2.2 Hz, 1H), 8.30 (s, 1H), 8.19-8.12 (m, 1H), 8.11-8.02 (m, 2H), 7.66-7.56 (m, 1H), 7.39-7.33 (m, 2H), 7.21-7.13 (m, 3H), 6.29-6.19 (m, 1H), 6.16-6.05 (m, 1H), 5.02-4.87 (m, 1H), 4.56-4.41 (m, 2H), 4.30-4.18 (m, 1H), 4.17-4.09 (m, 1H), 3.96-3.74 (m, 5H), 2.28-2.17 (m, 2H), 2.07-1.97 (m, 1H), 1.92-1.63 (m, 9H), 1.57-1.38 (m, 4H), 1.13 (d, J = 7.1 Hz, 3H), 0.85- 0.76 (m, 4H), 0.73- 0.63 (m, 1H) |
| 371 | propyl (((R)-(2- (((3S,6S,9aS)-3- ((6S,7R)-6-(5- chloropyridin-3-yl)- 7-cyano-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-6-(5- chloropyridin-3-yl)- 7-cyano-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 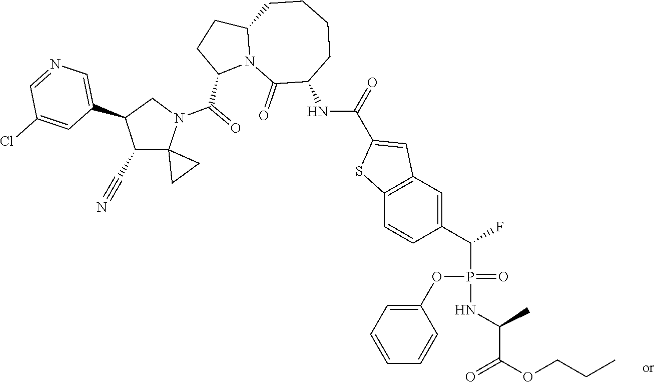 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cncc(Cl)c6)[C@@H](C#N)C56CC6)N4C3=O)cc2c1] 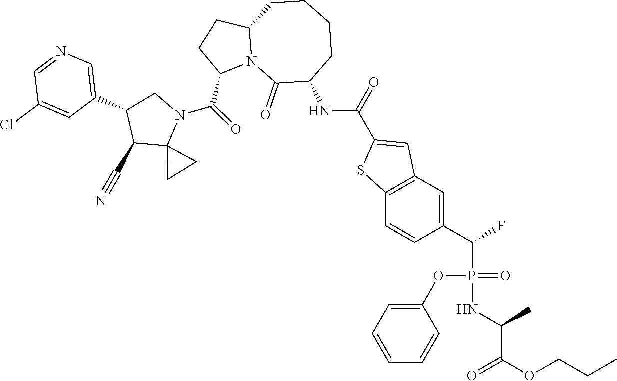 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cncc(Cl)c6)[C@H](C#N)C56CC6)N4C3=O)cc2c1] Pyrrolidine + Core SFC peak 1 was used for synthesis and biological testing | 903.2 | ¹H NMR (400 MHz, CDCl₃) δ 8.79 (s, 1H), 8.68-8.51 (m, 1H), 8.13 (s, 1H), 8.00-7.91 (m, 1H), 7.90-7.77 (m, 2H), 7.60-7.51 (m, 1H), 7.42-7.32 (m, 3H), 7.26-7.10 (m, 2H), 7.08-7.02 (m, 1H), 6.14-5.87 (m, 1H), 5.23-5.07 (m, 1H), 4.67-4.40 (m, 2H), 4.39-4.29 (m, 1H), 4.25-3.62 (m, 6H), 3.16 (s, 1H), 2.37- 1.76 (m, 12H), 1.72- 1.50 (m, 4H), 1.30 (d, J = 7.0 Hz, 2H), 1.15 (d, J = 6.9 Hz, 1H), 1.06-0.83 (m, 4H), 0.70-0.54 (m, 1H) |
| 372 | propyl (((R)-(2- (((3S,6S,10aS)-3- ((6R,7S)-7-cyano- 6-(5- methoxypyridin-3- yl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6S,7R)-7-cyano- 6-(5- methoxypyridin-3- yl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 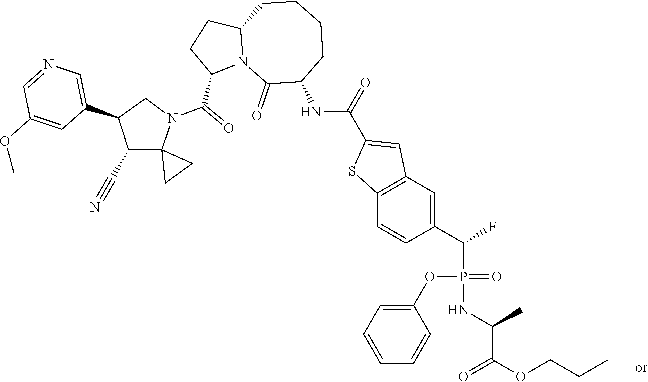 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cncc(OC)c6)[C@@H](C#N)C56CC6)N4C3=O)cc2c1] 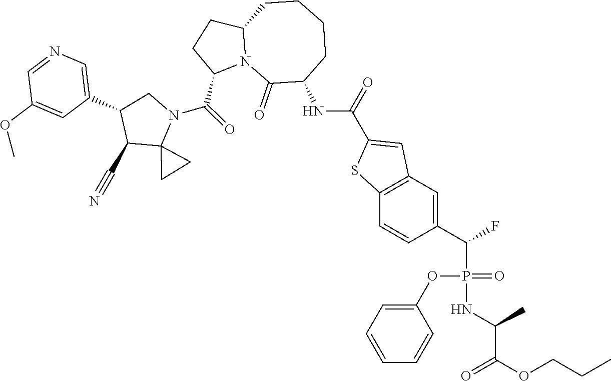 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cncc(OC)c6)[C@H](C#N)C56CC6)N4C3=O)cc2c1] Pyrrolidine + Core SFC peak 1 was used for synthesis and biological testing | 899 | ¹H NMR (400 MHz, DMSO) δ 8.89-8.81 (m, 1H), 8.30-8.25 (m, 2H), 8.21-8.18 (m, 1H), 8.10-8.04 (m, 2H), 7.63-7.54 (m, 2H), 7.39-7.33 (m, 2H), 7.17 (dd, J = 13.8, 7.7 Hz, 3H), 6.27-6.20 (m, 1H), 6.16-6.05 (m, 1H), 4.97-4.87 (m, 1H), 4.55-4.46 (m, 1H), 4.30-4.16 (m, 2H), 4.09-4.01 (m, 1H), 3.97-3.76 (m, 8H), 2.36-2.24 (m, 1H), 2.08-1.98 (m, 2H), 1.90-1.38 (m, 13H), 1.13 (d, J = 7.1 Hz, 3H), 0.85-0.74 (m, 4H), 0.65-0.55 (m, 1H) |
| 373 | (4-methyl- 112,212,4- triazolidin-3- yl)methyl (((1R)- fluoro(2- (((3S,6S,9aS)-5- oxo-3-((R)-6- (pyridin-3-yl)-4- azaspiro[2.4]heptane- 4- carbonyl)octahydro- 1H-pyrrolo[1,2- a]azepin-6- yl)carbamoyl)-2,3- dihydrobenzo[b] thiophen-5- yl)methyl)(phenoxy) phosphoryl)-L- alaninate | 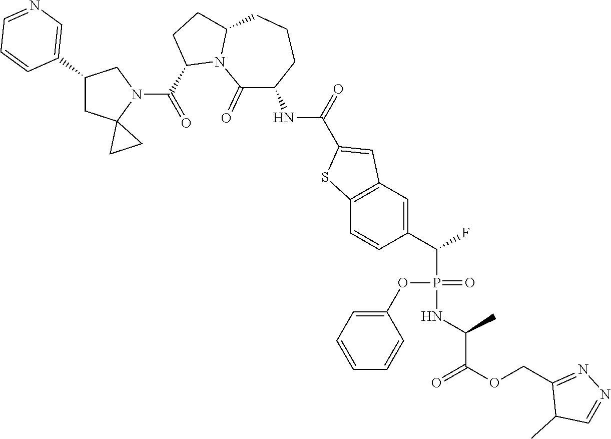 [SMILES: CC1C=NN=C1COC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cccnc6)CC56CC6)N4C3=O)cc2c1] | 883.2 | ¹H NMR (400 MHz, DMSO) δ 8.80 (d, J = 6.7 Hz, 1H), 8.60 (s, 1H), 8.53-8.40 (m, 2H), 8.28 (s, 1H), 8.14-8.00 (m, 2H), 7.85 (d, J = 7.1 Hz, 1H), 7.58 (d, J = 8.2 Hz, 1H), 7.42-7.25 (m, 3H), 7.20-7.05 (m, 3H), 6.40-6.05 (m, 2H), 5.24-5.17 (m, 1H), 4.71-4.55 (m, 2H), 4.14-4.04 (m, 1H), 4.02-3.94 (m, 1H), 3.78-3.70 (m, 1H), 3.62-3.58 (m, 3H), 3.31-3.22 (m, 1H), 3.11-3.02 (m, 2H), 2.25-1.94 (m, 6H), 1.83-1.65 (m, 6H), 1.29-1.24 (m, 2H), 1.18 (d, J = 6.0 Hz, 3H), 0.55- 0.40 (m, 2H) |
| 374 | oxetan-3-ylmethyl (((R)-fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 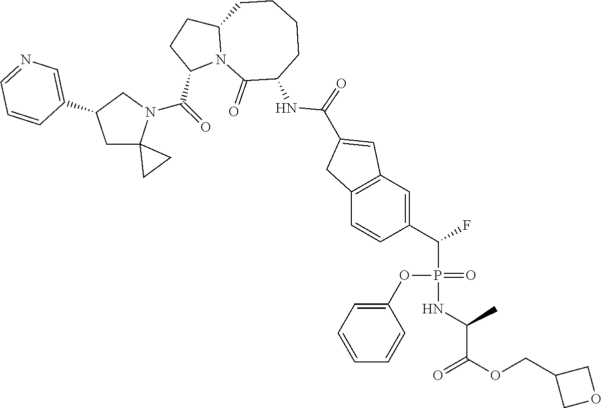 [SMILES: C[C@H](NP(=O)(Oc1ccccc1)[C@@H](F)c1ccc2c(c1)C=C(C(=O)N[C@H]1CCCC[C@H]3CC[C@@H](C(=O)N4C[C@@H](c5cccnc5)CC45CC5)N3C1=O)C2)C(=O)OCC1COC1] | 872.6 | ¹H NMR (400 MHz, DMSO) δ 8.92-8.79 (m, 1H), 8.64-8.54 (m, 1H), 8.48-8.38 (m, 1H), 8.33-8.24 (m, 1H), 8.12-8.03 (m, 2H), 7.85-7.74 (m, 1H), 7.67-7.53 (m, 1H), 7.41-7.28 (m, 3H), 7.24-7.10 (m, 3H), 6.33-6.09 (m, 2H), 5.03-4.87 (m, 1H), 4.62-4.44 (m, 3H), 4.32-3.96 (m, 6H), 3.91-3.70 (m, 2H), 3.65-3.48 (m, 1H), 3.20-2.99 (m, 1H), 2.37-1.40 (m, 16H), 1.21-1.05 (m, 3H), 0.57-0.34 (m, 2H) |
| 375 | 2-methoxyethyl (((R)-(2- (((3S,6S,10aS)-3- ((6S,7R)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 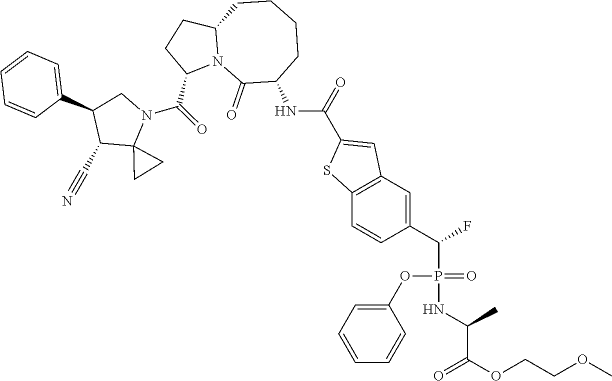 [SMILES: COCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6ccccc6)[C@@H](C#N)C56CC6)N4C3=O)cc2c1] | 884.3 | ¹H NMR (400 MHz, DMSO) δ 8.94-8.85 (m, 1H), 8.31 (s, 1H), 8.10-8.03 (m, 2H), 7.64-7.57 (m, 1H), 7.51-7.44 (m, 2H), 7.41-7.30 (m, 5H), 7.22-7.10 (m, 3H), 6.31-6.11 (m, 2H), 5.01-4.90 (m, 1H), 4.57-4.42 (m, 2H), 4.28-4.17 (m, 1H), 4.17-3.64 (m, 6H), 3.49-3.36 (m, 2H), 3.23-3.18 (m, 3H), 2.26-2.16 (m, 2H), 2.05-1.50 (m, 12H), 1.20-1.10 (m, 3H), 0.84-0.62 (m, 2H) |
| 376 | 2-methoxyethyl N- (((R)-(2- (((3S,6S,10aS)-3- ((6S,7R)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- O-methyl-L- serinate | 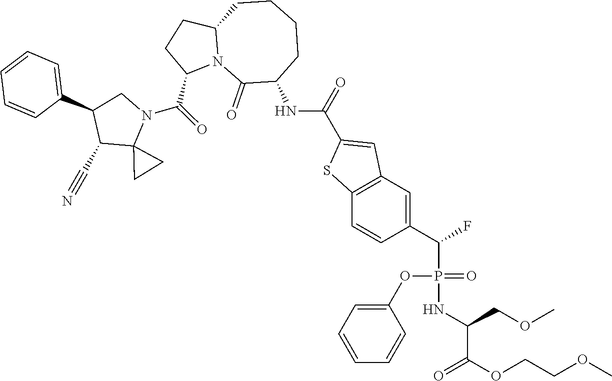 [SMILES: COCCOC(=O)[C@H](COC)NP(=O)(Oc1ccccc1)[C@@H](F)c1ccc2sc(C(=O)N[C@H]3CCCC[C@H]4CC[C@@H](C(=O)N5C[C@H](c6ccccc6)[C@@H](C#N)C56CC6)N4C3=O)cc2c1] | 914.2 | ¹H NMR (400 MHz, DMSO) δ 8.93-8.86 (m, 1H), 8.33-8.28 (m, 1H), 8.10-8.04 (m, 2H), 7.63-7.58 (m, 1H), 7.51-7.45 (m, 2H), 7.41-7.31 (m, 5H), 7.22-7.11 (m, 3H), 6.29-6.08 (m, 2H), 5.01-4.90 (m, 1H), 4.56-4.42 (m, 2H), 4.27-4.18 (m, 1H), 4.14-4.09 (m, 1H), 4.06-3.87 (m, 3H), 3.76-3.66 (m, 2H), 3.48-3.37 (m, 3H), 3.32-3.25 (m, 1H), 3.23-3.11 (m, 6H), 2.27-2.16 (m, 2H), 1.98-1.43 (m, 12H), 0.85-0.62 (m, 2H). |
| 377 | 2-methoxyethyl (((R)-(2- (((3S,6S,10aS)-3- ((6S,7R)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl)(2, 2,2- trifluoroethoxy) phosphoryl)-L- alaninate | 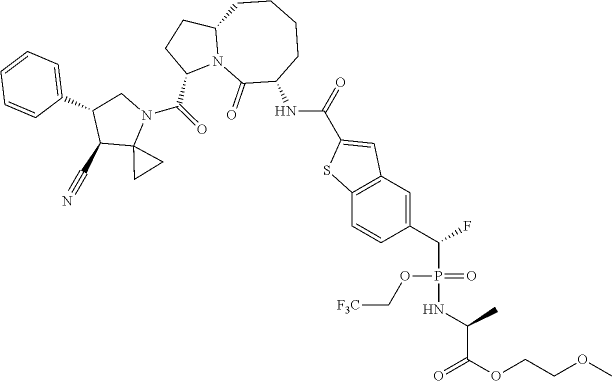 [SMILES: COCCOC(=O)[C@H](C)NP(=O)(OCC(F)(F)F)[C@@H](F)c1ccc2sc(C(=O)N[C@H]3CCCC[C@H]4CC[C@@H](C(=O)N5C[C@@H](c6ccccc6)[C@H](C#N)C56CC6)N4C3=O)cc2c1] | 890.2 | ¹H NMR (400 MHz, DMSO) δ 8.95-8.87 (m, 1H), 8.32-8.26 (m, 1H), 8.09-8.03 (m, 1H), 8.02-7.96 (m, 1H), 7.55-7.45 (m, 3H), 7.41-7.29 (m, 3H), 6.23-6.00 (m, 2H), 5.01-4.91 (m, 1H), 4.67-4.44 (m, 4H), 4.27-4.00 (m, 4H), 3.94-3.67 (m, 3H), 3.53-3.44 (m, 2H), 3.25-3.19 (m, 3H), 2.26-2.16 (m, 2H), 1.97-1.44 (m, 12H), 1.29-1.20 (m, 3H), 0.85-0.77 (m, 1H), 0.72-0.62 (m, 1H) |
| 378 | 2-methoxyethyl (((R)-fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 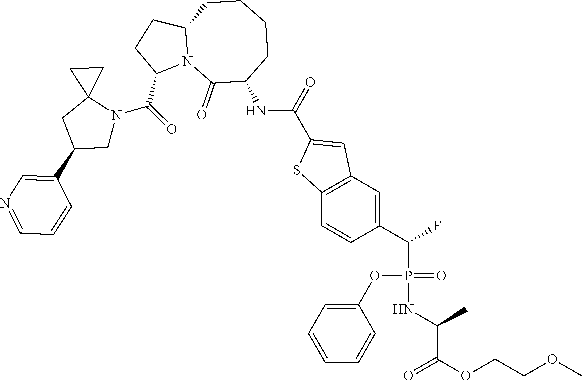 [SMILES: COCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | 860.3 | ¹H NMR (400 MHz, CDCl₃) δ 8.61-8.54 (m, 1H), 8.43-8.33 (m, 1H), 7.93-7.79 (m, 3H), 7.77-7.69 (m, 1H), 7.67-7.60 (m, 1H), 7.58-7.51 (m, 1H), 7.51-7.42 (m, 1H), 7.39-7.32 (m, 1H), 7.24-7.17 (m, 2H), 7.14-7.03 (m, 2H), 6.09-5.81 (m, 1H), 5.26-5.04 (m, 1H), 4.59-4.50 (m, 1H), 4.33-4.15 (m, 4H), 4.14-3.96 (m, 3H), 3.62-3.49 (m, 3H), 3.36-3.32 (m, 3H), 2.36-2.16 (m, 3H), 2.15-1.95 (m, 7H), 1.91-1.69 (m, 4H), 1.67-1.53 (m, 2H), 1.37-1.15 (m, 3H), 0.56-0.45 (m, 2H) |
| 379 | propyl (((R)- fluoro(2- (((3S,6S,9aS)-5- oxo-3-((R)-6- (pyridin-3-yl)-4- azaspiro[2.4]heptane- 4- carbonyl)octahydro- 1H-pyrrolo[1,2- a]azepin-6- yl)carbamoyl)benzo [b]thiophen-5- yl)methyl)(phenoxy) phosphoryl)-L- alaninate | 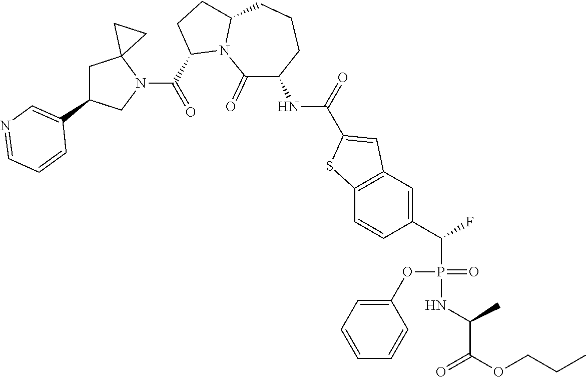 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cccnc6)CC56CC6)N4C3=O)cc2c1] | 830.4 | ¹H NMR (400 MHz, DMSO) δ 8.83-8.65 (m, 2H), 8.54 (d, J = 4.7 Hz, 1H), 8.27 (s, 1H), 8.10-8.00 (m, 3H), 7.64-7.49 (m, 2H), 7.41-7.33 (m, 2H), 7.21-7.11 (m, 3H), 6.31-6.02 (m, 2H), 4.68-4.56 (m, 2H), 4.16-3.69 (m, 7H), 2.25-1.43 (m, 16H), 1.19-1.08 (m, 3H), 0.88-0.74 (m, 3H), 0.56-0.41 (m, 2H) |
| 380 | propyl (((R)-(2- (((3S,6S,10aS)-3- ((6R,7S)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 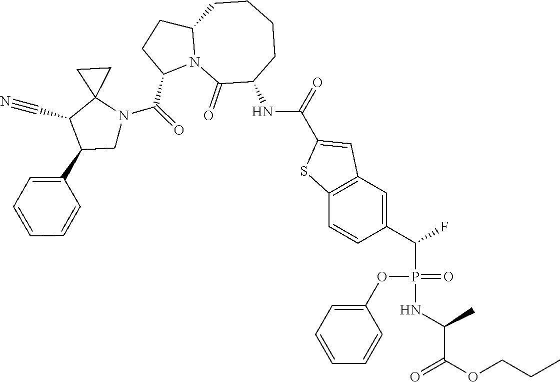 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6ccccc6)[C@H](C#N)C56CC6)N4C3=O)cc2c1] | 868.4 | ¹H NMR (400 MHz, CDCl₃) δ 8.00-7.90 (m, 1H), 7.88-7.80 (m, 1H), 7.78 (d, J = 2.2 Hz, 1H), 7.61- 7.49 (m, 1H), 7.42- 7.27 (m, 8H), 7.25- 7.02 (m, 3H), 6.10- 5.81 (m, 1H), 5.19- 5.03 (m, 1H), 4.80- 4.65 (m, 1H), 4.58- 4.42 (m, 1H), 4.35- 4.21 (m, 1H), 4.14- 3.94 (m, 3H), 3.86- 3.46 (m, 4H), 2.49- 2.34 (m, 1H), 2.21- 1.89 (m, 8H), 1.84- 1.74 (m, 3H), 1.62- 1.55 (m, 2H), 1.36- 1.06 (m, 6H), 0.95- 0.82 (m, 3H), 0.79- 0.67 (m, 1H) |
| 381 | propyl (((R)-(2- (((3S,6S,10aS)-3- ((6S,7R)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 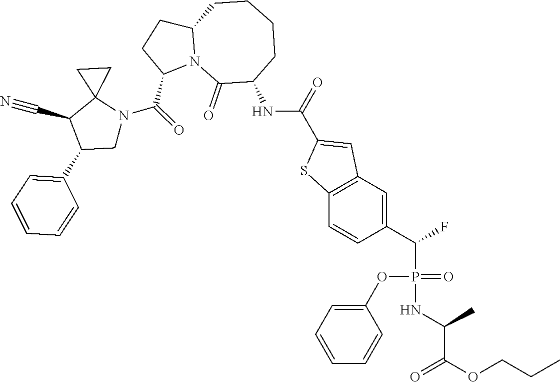 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6ccccc6)[C@@H](C#N)C56CC6)N4C3=O)cc2c1] | 868.4 | ¹H NMR (400 MHz, CDCl₃) δ 7.96 (d, J = 15.1 Hz, 1H), 7.86 (d, J = 8.4 Hz, 1H), 7.79 (s, 1H), 7.56 (dd, J = 24.2, 8.8 Hz, 1H), 7.43-7.27 (m, 8H), 7.25-7.02 (m, 3H), 6.11-5.83 (m, 1H), 5.13 (s, 1H), 4.57-4.43 (m, 1H), 4.35-4.24 (m, 1H), 4.22-3.95 (m, 5H), 3.78-3.53 (m, 2H), 3.48-3.40 (m, 1H), 2.59-2.47 (m, 1H), 2.26-2.04 (m, 5H), 1.85-1.72 (m, 4H), 1.67-1.56 (m, 4H), 1.40-1.03 (m, 6H), 0.94-0.69 (m, 4H) |
| 382 | propyl (((R)-(2- (((3S,6S,9aS)-3- ((6R,7S)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 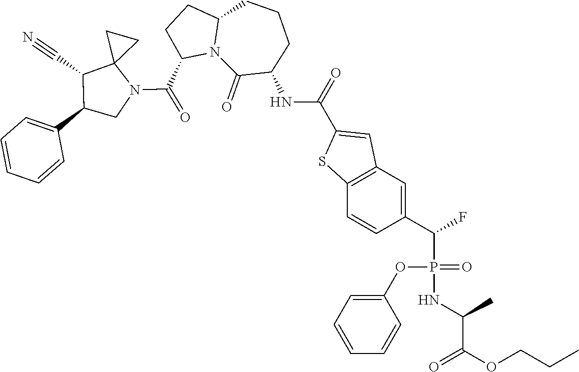 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6ccccc6)[C@H](C#N)C56CC6)N4C3=O)cc2c1] | 854.5 | ¹H NMR (400 MHz, DMSO) δ 8.83-8.72 (m, 1H), 8.30-8.22 (m, 1H), 8.12-8.02 (m, 2H), 7.67-7.57 (m, 1H), 7.53-7.45 (m, 2H), 7.42-7.26 (m, 5H), 7.24-7.10 (m, 3H), 6.34-5.99 (m, 2H), 4.69-4.55 (m, 2H), 4.19-4.06 (m, 1H), 4.03-3.71 (m, 7H), 2.25-2.02 (m, 3H), 2.00-1.60 (m, 9H), 1.57-1.36 (m, 2H), 1.20-1.10 (m, 3H), 0.88-0.58 (m, 5H) |
| 383 | propyl (((R)-(2- (((3S,6S,9aS)-3- ((6S,7R)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 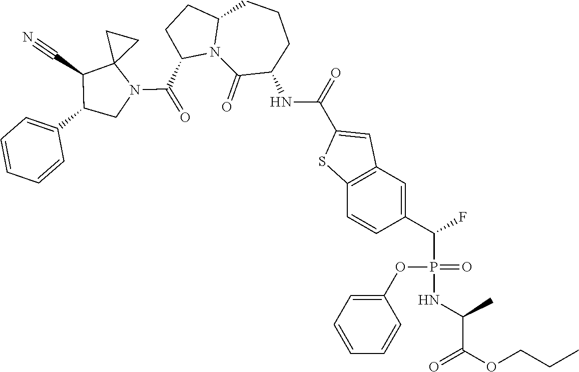 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6ccccc6)[C@@H](C#N)C56CC6)N4C3=O)cc2c1] | 854.5 | ¹H NMR (400 MHz, DMSO) δ 8.86-8.75 (m, 1H), 8.30-8.24 (m, 1H), 8.12-8.02 (m, 2H), 7.65-7.57 (m, 1H), 7.56-7.45 (m, 2H), 7.44-7.28 (m, 5H), 7.25-7.09 (m, 3H), 6.28-6.02 (m, 2H), 4.73-4.57 (m, 2H), 4.40-4.27 (m, 1H), 4.07-3.61 (m, 7H), 2.25-2.10 (m, 2H), 2.10-1.61 (m, 10H), 1.58-1.33 (m, 2H), 1.21-1.06 (m, 3H), 0.90-0.60 (m, 5H) |
| 384 | propyl N-(((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-O- methyl-L-serinate | 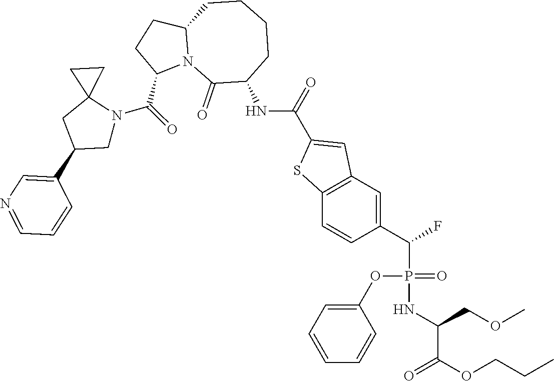 [SMILES: CCCOC(=O)[C@H](COC)NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | 874.2 | ¹H NMR (400 MHz, DMSO) δ 8.87-8.79 (m, 1H), 8.61 (mi, 1H), 8.46 (d, J = 4.7 Hz, 1H), 8.29 (s, 1H), 8.10-8.01 (m, 2H), 7.86 (d, J = 7.9 Hz, 1H), 7.60 (d, J = 8.4 Hz, 1H), 7.42- 7.30 (m, 3H), 7.23- 7.10 (m, 3H), 6.26- 6.00 (m, 2H), 4.99- 4.89 (m, 1H), 4.56- 4.47 (m, 1H), 4.28- 4.11 (m, 2H), 4.02- 3.77 (m, 4H), 3.74- 3.55 (m, 2H), 3.25- 3.20 (m, 1H), 3.17- 3.10 (m, 3H), 2.34- 2.22 (m, 1H), 2.18- 2.11 (m, 2H), 2.06- 1.95 (m, 3H), 1.92- 1.71 (m, 6H), 1.66- 1.46 (m, 5H), 1.41- 1.30 (m, 1H), 0.86- 0.71 (m, 3H), 0.53- 0.40 (m, 2H) |
| 385 | pyridin-2-ylmethyl (((R)-fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 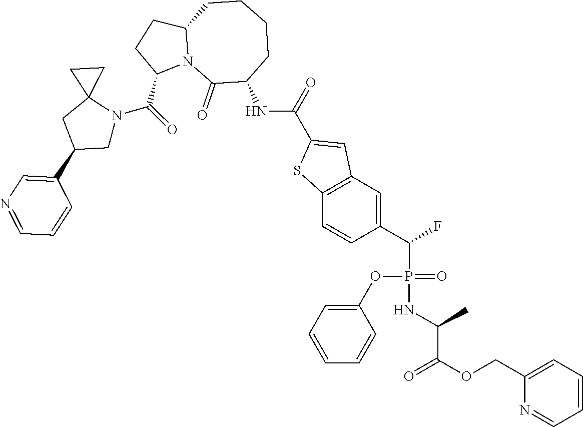 [SMILES: C[C@H](NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1)C(=O)OCc1ccccn1] | 893.9 | ¹H NMR (400 MHz, DMSO) δ 8.87-8.79 (m, 1H), 8.60-8.55 (m, 1H), 8.54-8.49 (m, 1H), 8.44-8.40 (m, 1H), 8.30-8.26 (m, 1H), 8.08-8.01 (m, 2H), 7.79-7.72 (m, 2H), 7.62-7.56 (m, 1H), 7.36-7.26 (m, 5H), 7.19-7.10 (m, 3H), 6.36-6.12 (m, 2H), 5.15-4.90 (m, 3H), 4.56-4.48 (m, 1H), 4.28-4.10 (m, 2H), 4.06-3.78 (m, 2H), 3.61-3.52 (m, 1H), 2.32-2.23 (m, 1H), 2.19-1.98 (m, 5H), 1.92-1.69 (m, 6H), 1.66-1.44 (m, 4H), 1.24-1.17 (m, 3H), 0.52-0.38 (m, 2H) |
| 386 | (3-methyloxetan-3- yl)methyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 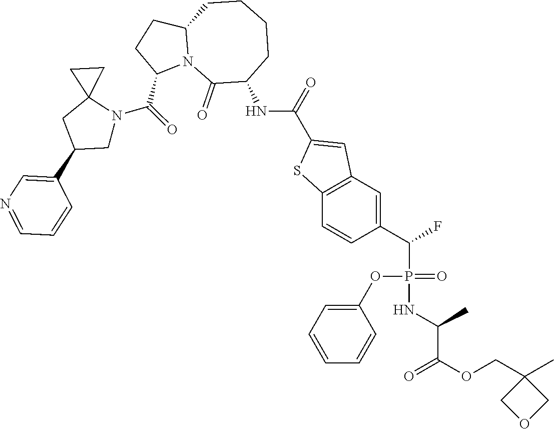 [SMILES: C[C@H](NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1)C(=O)OCC1(C)COC1] | 886.4 | ¹H NMR (400 MHz, DMSO) δ 8.96-8.79 (m, 1H), 8.66-8.50 (m, 1H), 8.48-8.37 (m, 1H), 8.34-8.24 (m, 1H), 8.23-7.93 (m, 2H), 7.85-7.72 (m, 1H), 7.66-7.50 (m, 1H), 7.46-7.07 (m, 5H), 6.39-5.91 (m, 2H), 5.10-4.84 (m, 1H), 4.64-4.43 (m, 1H), 4.37-3.97 (m, 7H), 3.94-3.69 (m, 3H), 3.63-3.56 (m, 1H), 2.34-1.46 (m, 16H), 1.29-0.95 (m, 6H), 0.68-0.14 (m, 2H) |
| 387 | propyl (((S)-(2- (((3S,6S,9aS)-3- ((6R,7S)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 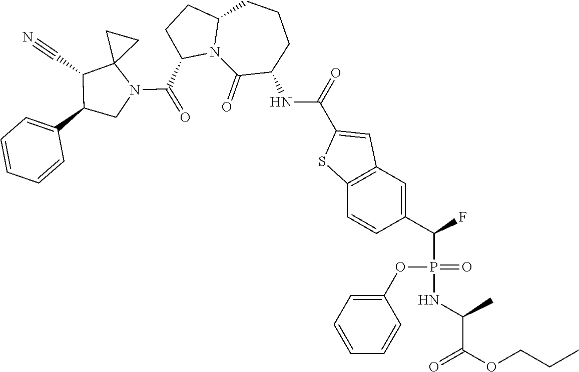 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5C[C@@H](c6ccccc6)[C@H](C#N)C56CC6)N4C3=O)cc2c1] | 854.3 | ¹H NMR (400 MHz, DMSO) δ 8.88-8.73 (m, 1H), 8.31-8.24 (m, 1H), 8.13-8.03 (m, 2H), 7.66-7.58 (m, 1H), 7.53-7.44 (m, 2H), 7.41-7.27 (m, 5H), 7.24-7.09 (m, 3H), 6.33-6.06 (m, 2H), 4.69-4.55 (m, 2H), 4.19-4.07 (m, 1H), 4.01-3.63 (m, 7H), 2.26-2.02 (m, 3H), 2.01-1.59 (m, 9H), 1.57-1.40 (m, 2H), 1.22-0.92 (m, 3H), 0.87-0.76 (m, 4H), 0.72-0.61 (m, 1H) |
| 388 | propyl (((S)-(2- (((3S,6S,9aS)-3- ((6S,7R)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 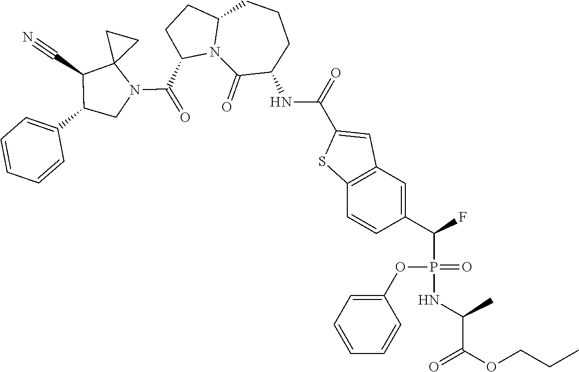 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5C[C@H](c6ccccc6)[C@@H](C#N)C56CC6)N4C3=O)cc2c1] | 854.3 | ¹H NMR (400 MHz, DMSO) δ 8.89-8.79 (m, 1H), 8.33-8.23 (m, 1H), 8.15-8.03 (m, 2H), 7.66-7.57 (m, 1H), 7.55-7.47 (m, 2H), 7.44-7.29 (m, 5H), 7.26-7.09 (m, 3H), 6.32-6.06 (m, 2H), 4.72-4.58 (m, 2H), 4.40-4.27 (m, 1H), 4.07-3.66 (m, 7H), 2.26-2.13 (m, 2H), 2.10-1.90 (m, 2H), 1.89-1.62 (m, 8H), 1.57-1.40 (m, 2H), 1.21-0.92 (m, 3H), 0.88-0.62 (m, 5H) |
| 389 | ethyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- phenylalaninate | 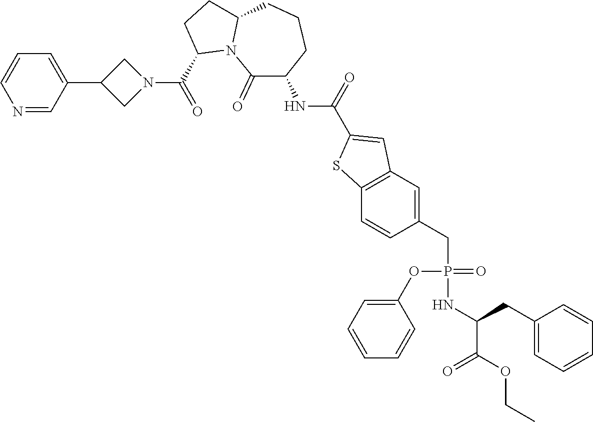 [SMILES: CCOC(=O)[C@H](Cc1ccccc1)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 834.1 | ¹H NMR (400 MHz, CDCl₃) δ 9.35-8.98 (m, 1H), 8.76-8.12 (m, 4H), 7.92-7.51 (m, 4H), 7.37-7.20 (m, 4H), 7.08-6.67 (m, 6H), 5.03-3.76 (m, 13H), 3.31-3.10 (m, 2H), 2.90-2.68 (m, 2H), 2.26-2.04 (m, 4H), 1.96-1.83 (m, 4H), 1.07-0.99 (m, 3H) |
| 390 | benzyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- leucinate | 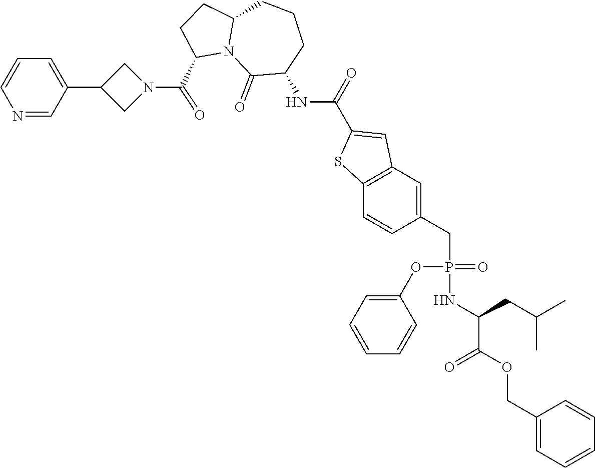 [SMILES: CC(C)C[C@H](NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1)C(=O)OCc1ccccc1] | 862.2 | ¹H NMR (400 MHz, DMSO) δ 8.85-8.70 (m, 1H), 8.59-8.38 (m, 2H), 8.20 (s, 1H), 7.95-7.82 (m, 3H), 7.45-7.25 (m, 9H), 7.16-7.09 (m, 3H), 5.74-5.51 (m, 1H), 5.05-4.89 (m, 2H), 4.85-4.58 (m, 2H), 4.48-4.19 (m, 3H), 4.05-3.83 (m, 3H), 3.82-3.69 (m, 1H), 3.52-3.41 (m, 1H), 2.26-2.13 (m, 1H), 2.08-1.92 (m, 2H), 1.89-1.65 (m, 7H), 1.35-1.03 (m, 3H), 0.69-0.40 (m, 6H) |
| 391 | isopropyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- prolinate | 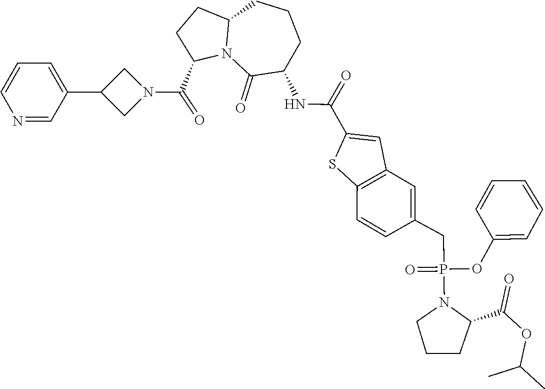 [SMILES: CC(C)OC(=O)[C@@H]1CCCN1P(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 798.2 | ¹H NMR (400 MHz, DMSO) δ 8.85-8.70 (m, 1H), 8.60-8.40 (m, 2H), 8.22 (s, 1H), 8.01-7.84 (m, 3H), 7.51-7.05 (m, 7H), 4.93-4.58 (m, 3H), 4.47-3.80 (m, 7H), 3.70-3.49 (m, 2H), 3.44-3.37 (m, 1H), 3.15-2.96 (m, 1H), 2.25-2.14 (m, 1H), 2.08-1.64 (m, 13H), 1.20-1.04 (m, 6H) |
| 392 | isopropyl O- methyl-N-(((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- serinate | 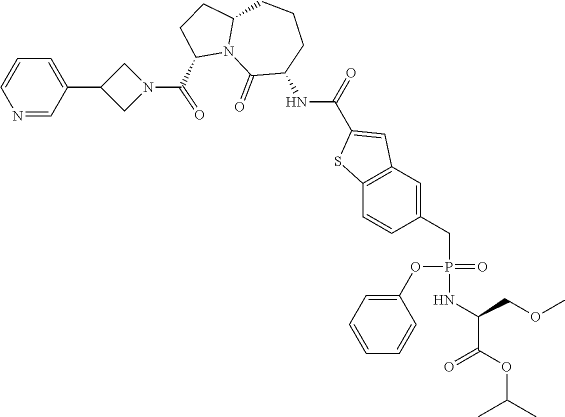 [SMILES: COC[C@H](NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1)C(=O)OC(C)C] | 802.3 | ¹H NMR (400 MHz, CDCl₃) δ 8.89-8.39 (m, 2H), 8.09-7.69 (m, 4H), 7.59-7.32 (m, 2H), 7.26-7.20 (m, 2H), 7.14-6.99 (m, 3H), 5.00-4.85 (m, 1H), 4.76-4.29 (m, 4H), 4.20-4.06 (m, 1H), 4.03-3.97 (m, 1H), 3.95-3.79 (m, 2H), 3.56-3.33 (m, 4H), 3.15-3.00 (m, 4H), 2.32-1.37 (m, 10H), 1.17-1.06 (m, 6H) |
| 393 | 2-methoxyethyl (((2-(((3S,6S,9aS)- 5-oxo-3-(3- (pyridin-3- yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 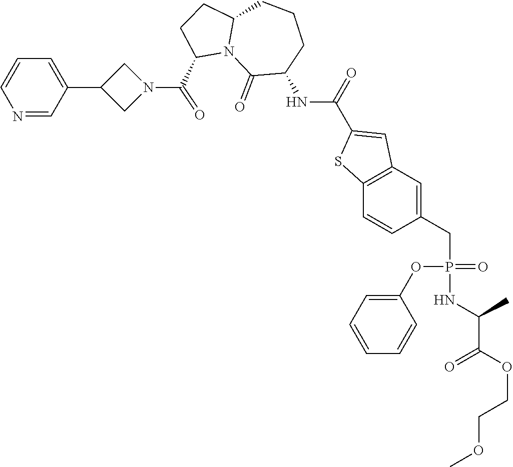 [SMILES: COCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 788.2 | ¹H NMR (400 MHz, CDCl₃) δ 8.93-8.47 (m, 2H), 8.19-7.30 (m, 7H), 7.27-7.20 (m, 2H), 7.12-7.03 (m, 3H), 5.05-4.32 (m, 5H), 4.21-4.06 (m, 3H), 4.00-3.85 (m, 3H), 3.47-3.33 (m, 4H), 3.28-3.23 (m, 3H), 2.23-1.60 (m, 10H), 1.21-1.05 (m, 3H) |
| 394 | benzyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 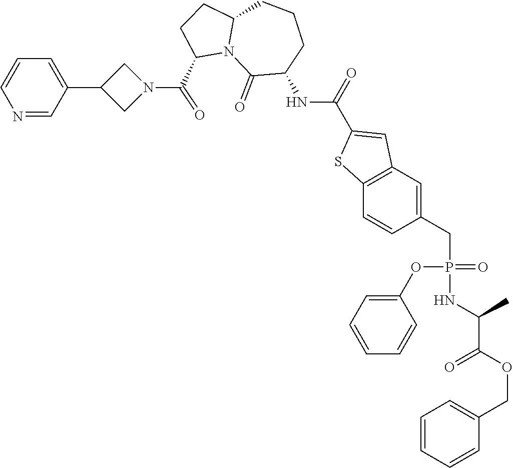 [SMILES: C[C@H](NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1)C(=O)OCc1ccccc1] | 820.2 | ¹H NMR (400 MHz, DMSO) δ 8.86-8.37 (m, 3H), 8.25-8.12 (m, 1H), 7.96-7.80 (m, 3H), 7.49-7.24 (m, 9H), 7.22-7.05 (m, 3H), 5.82-5.61 (m, 1H), 5.10-4.96 (m, 2H), 4.85-4.57 (m, 2H), 4.48-4.19 (m, 3H), 4.05-3.81 (m, 4H), 3.48-3.44 (m, 1H), 2.25-2.14 (m, 1H), 2.09-1.94 (m, 2H), 1.93-1.62 (m, 7H), 1.15-0.98 (m, 3H) |
| 395 | isopropyl 2-methyl- 2-((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino) propanoate | 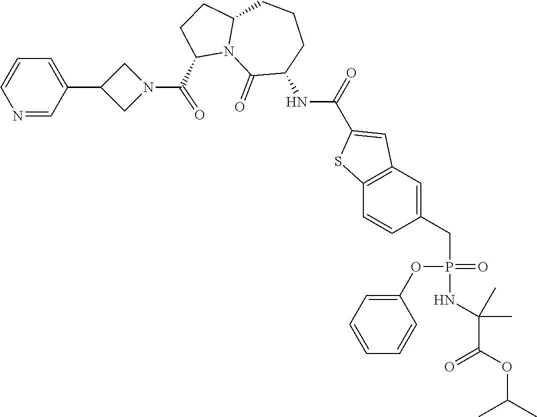 [SMILES: CC(C)OC(=O)C(C)(C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 786.4 | ¹H NMR (400 MHz, CDCl₃) δ 9.06-8.22 (m, 2H), 8.19-7.64 (m, 4H), 7.61-7.31 (m, 2H), 7.25-7.19 (m, 3H), 7.18-7.00 (m, 3H), 5.06-4.87 (m, 1H), 4.84-4.33 (m, 4H), 4.20-3.81 (m, 3H), 3.63-3.33 (m, 3H), 2.25-1.72 (m, 10H), 1.40-1.25 (m, 6H), 1.20-1.10 (m, 6H) |
| 396 | ethyl (((7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 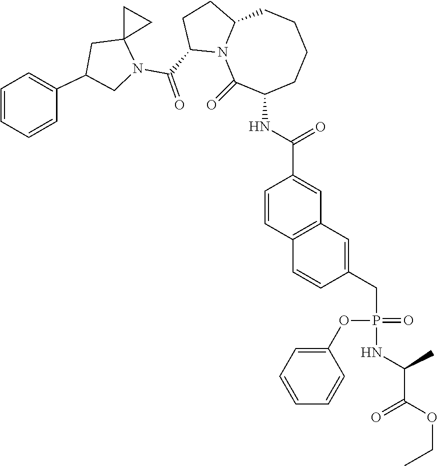 [SMILES: CCOC(=O)[C@H](C)NP(=O)(Cc1ccc2ccc(C(=O)N[C@H]3CCCC[C@H]4CC[C@@H](C(=O)N5CC(c6ccccc6)CC56CC6)N4C3=O)cc2c1)Oc1ccccc1] | 805.3 | ¹H NMR (400 MHz, DMSO) δ 8.36-8.28 (m, 1H), 7.95-7.80 (m, 4H), 7.67-7.56 (m, 1H), 7.51-7.41 (m, 1H), 7.37-7.29 (m, 5H), 7.28-7.08 (m, 5H), 5.28-5.17 (m, 1H), 4.62-4.56 (m, 1H), 4.38-4.26 (m, 1H), 4.15-3.46 (m, 7H), 3.39-3.06 (m, 1H), 2.60 (d, J = 9.1 Hz, 1H), 2.49- 2.38 (m, 1H), 2.29- 1.95 (m, 9H), 1.92- 1.60 (m, 6H), 1.23 1.10 (m, 5H), 0.65 0.48 (m, 2H) |
| 397 | benzyl (((7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 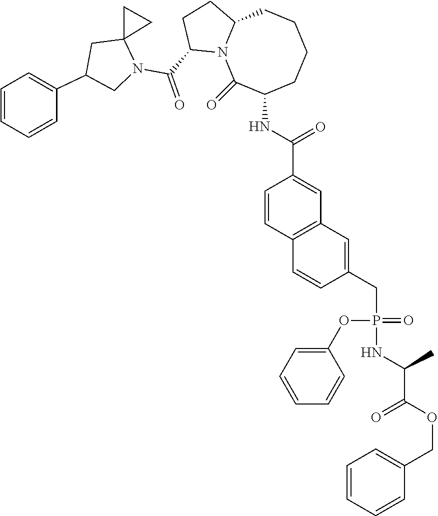 [SMILES: C[C@H](NP(=O)(Cc1ccc2ccc(C(=O)N[C@H]3CCCC[C@H]4CC[C@@H](C(=O)N5CC(c6ccccc6)CC56CC6)N4C3=O)cc2c1)Oc1ccccc1)C(=O)OCc1ccccc1] | 867.4 | ¹H NMR (400 MHz, CDCl₃) δ 8.24 (s, 1H), 7.84-7.69 (m, 4H), 7.53-7.45 (m, 1H), 7.29-7.21 (m, 6H), 7.20-7.12 (m, 7H), 7.10-6.98 (m, 3H), 5.20-5.09 (m, 1H), 5.04-4.84 (m, 2H), 4.55-4.45 (m, 1H), 4.31-4.17 (m, 1H), 4.06-3.83 (m, 2H), 3.61-3.39 (m, 3H), 3.33-3.00 (m, 1H), 2.45-2.29 (m, 2H), 2.19-2.10 (m, 2H), 2.03-1.87 (m, 5H), 1.76-1.53 (m, 5H), 1.12-1.04 (m, 3H), 0.86-0.72 (m, 2H), 0.56-0.38 (m, 2H) |
| 398 | phenyl P-((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)-N-((S)- 2- oxotetrahydrofuran- 3- yl)phosphonamidate | 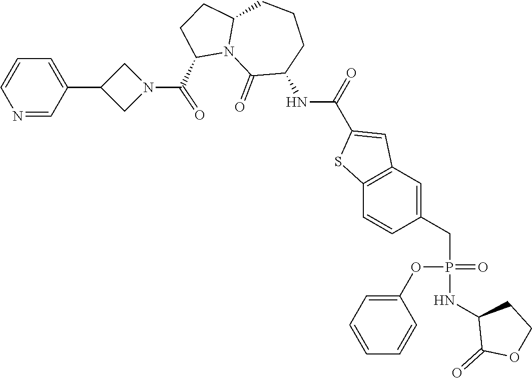 [SMILES: O=C(N[C@H]1CCC[C@H]2CC[C@@H](C(=O)N3CC(c4cccnc4)C3)N2C1=O)c1cc2cc(CP(=O)(N[C@H]3CCOC3=O)Oc3ccccc3)ccc2s1] | 742.2 | ¹H NMR (400 MHz, CDCl₃) δ 8.93-8.27 (m, 2H), 8.27-7.48 (m, 5H), 7.44-7.20 (m, 4H), 7.19-7.00 (m, 3H), 5.01-4.44 (m, 3H), 4.42-3.72 (m, 7H), 3.66-3.34 (m, 3H), 2.40-1.59 (m, 12H) |
| 399 | diisopropyl 2,2′- ((((2-(((3S,6S,9aS)- 5-oxo-3-(3- (pyridin-3- yl)azetidine-1- carbonyl)octahydro- 1H-pyrrolo[1,2- a]azepin-6- yl)carbamoyl)benzo [b]thiophen-5- yl)methyl)phosphoryl) bis(azanediyl)) (2S,2′S)-dipropionate | 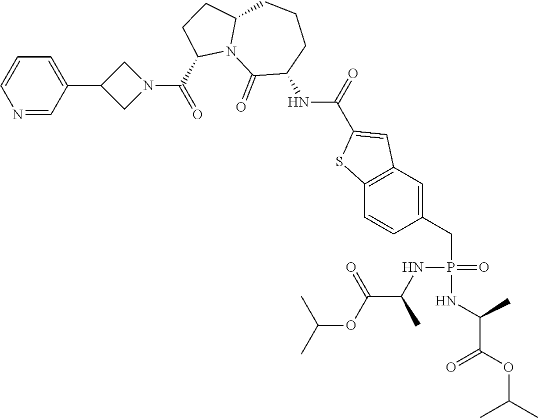 [SMILES: CC(C)OC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)N[C@@H](C)C(=O)OC(C)C] | 809.4 | ¹H NMR (400 MHz, DMSO) δ 8.87-8.63 (m, 3H), 8.45-8.30 (m, 1H), 8.21-8.13 (m, 1H), 7.93-7.68 (m, 3H), 7.43-7.35 (m, 1H), 4.94-4.76 (m, 2H), 4.73-4.23 (m, 6H), 4.16-3.88 (m, 3H), 3.85-3.54 (m, 2H), 3.29-3.04 (m, 2H), 2.27-1.62 (m, 10H), 1.28-1.04 (m, 18H) (TFA salt) |
| 400 | propyl ((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 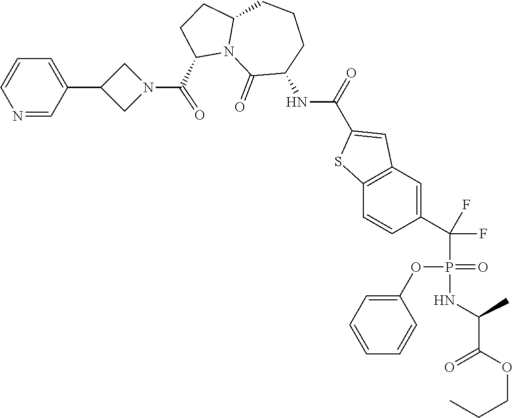 [SMILES: CCCOC(=O)[C@H](C)NP(=O)(Oc1ccccc1)C(F)(F)c1ccc2sc(C(=O)N[C@H]3CCC[C@H]4CC[C@@H](C(=O)N5CC(c6cccnc6)C5)N4C3=O)cc2c1] | 808.3 | ¹H NMR (400 MHz, DMSO) δ 8.99-8.78 (m, 1H), 8.57 (s, 1H), 8.52-8.40 (m, 1H), 8.38-8.30 (m, 1H), 8.26-8.12 (m, 2H), 7.91-7.80 (m, 1H), 7.67 (d, J = 8.7 Hz, 1H), 7.47-7.28 (m, 3H), 7.25-7.05 (m, 3H), 6.87-6.69 (m, 1H), 4.85-4.59 (m, 2H), 4.48-4.20 (m, 3H), 4.09-3.70 (m, 6H), 2.26-2.14 (m, 1H), 2.11-1.94 (m, 2H), 1.89-1.66 (m, 7H), 1.55-1.42 (m, 2H), 1.26-1.13 (m, 3H), 0.87-0.77 (m, 3H) |
| 401 | benzyl (2S)-2-((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino)- 4-phenylbutanoate | 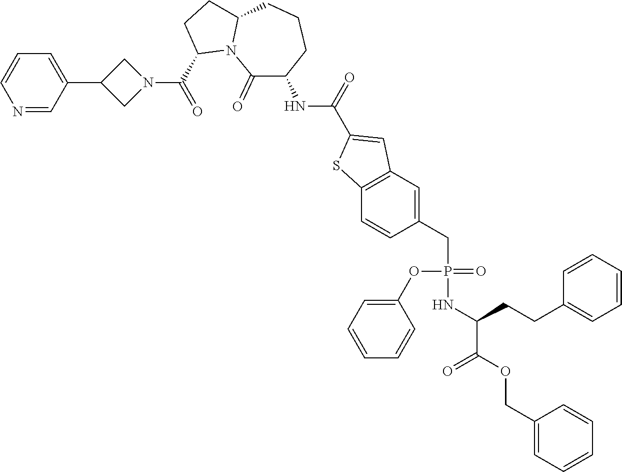 [SMILES: O=C(N[C@H]1CCC[C@H]2CC[C@@H](C(=O)N3CC(c4cccnc4)C3)N2C1=O)c1cc2cc(CP(=O)(N[C@@H](CCc3ccccc3)C(=O)OCc3ccccc3)Oc3ccccc3)ccc2s1] | 910.4 | ¹H NMR (400 MHz, DMSO) δ 8.83-8.69 (m, 1H), 8.64-8.38 (m, 2H), 8.24-8.16 (m, 1H), 7.95-7.79 (m, 3H), 7.50-7.24 (m, 9H), 7.23-7.02 (m, 6H), 6.93-6.87 (m, 1H), 6.79-6.71 (m, 1H), 5.89-5.65 (m, 1H), 5.08-4.89 (m, 2H), 4.83-4.56 (m, 2H), 4.49-4.18 (m, 3H), 4.06-3.75 (m, 4H), 3.51-3.42 (m, 2H), 2.38-2.09 (m, 3H), 2.06-1.51 (m, 11H) |
| 402 | propyl (((7- (((3S,6S,10aS)-5- oxo-3-((S)-6-(2- oxo-1,2- dihydropyridin-4- yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 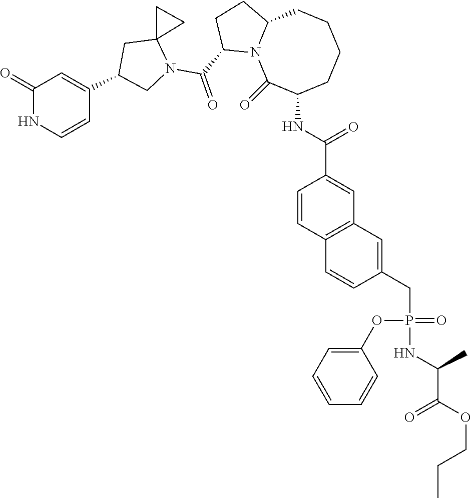 [SMILES: CCCOC(=O)[C@H](C)NP(=O)(Cc1ccc2ccc(C(=O)N[C@H]3CCCC[C@H]4CC[C@@H](C(=O)N5C[C@H](c6cc[nH]c(=O)c6)CC56CC6)N4C3=O)cc2c1)Oc1ccccc1] | 836.4 | ¹H NMR (400 MHz, DMSO) δ 11.43 (s, 1H), 8.71-8.56 (m, 1H), 8.45 (s, 1H), 8.03-7.83 (m, 4H), 7.60 (d, J = 8.4 Hz, 1H), 7.40-7.23 (m, 3H), 7.18-7.06 (m, 3H), 6.24 (s, 1H), 6.18 (d, J = 6.7 Hz, 1H), 5.76-5.59 (m, 1H), 5.05-4.96 (m, 1H), 4.60-4.47 (m, 1H), 4.39-4.20 (m, 2H), 3.91-3.84 (m, 2H), 3.83-3.76 (m, 1H), 3.59-3.54 (m, 2H), 3.51-3.49 (m, 1H), 2.27-2.12 (m, 2H), 2.11-2.00 (m, 2H), 1.99-1.86 (m, 4H), 1.86-1.66 (m, 4H), 1.66-1.36 (m, 6H), 1.11-1.05 (m, 3H), 0.84-0.75 (m, 3H), 0.55-0.37 (m, 2H) |
| 403 | propyl ((difluoro(7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 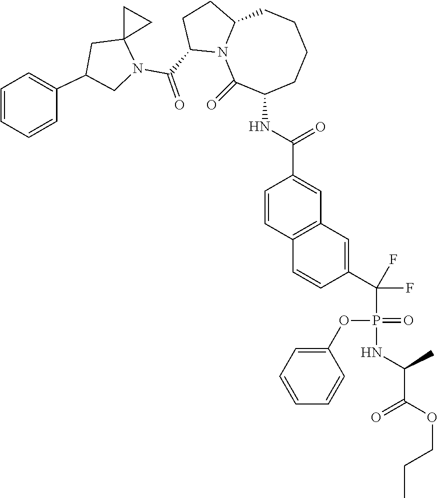 [SMILES: CCCOC(=O)[C@H](C)NP(=O)(Oc1ccccc1)C(F)(F)c1ccc2ccc(C(=O)N[C@H]3CCCC[C@H]4CC[C@@H](C(=O)N5CC(c6ccccc6)CC56CC6)N4C3=O)cc2c1] | 855.5 | ¹H NMR (400 MHz, DMSO) δ 8.81-8.70 (m, 2H), 8.44-8.35 (m, 1H), 8.22 (t, J = 7.5 Hz, 1H), 8.17 (s, 2H), 7.88 (d, J = 8.6 Hz, 1H), 7.49-7.35 (m, 6H), 7.33-7.20 (m, 4H), 6.99-6.86 (m, 1H), 5.19-5.01 (m, 1H), 4.68-4.17 (m, 3H), 4.10-3.54 (m, 5H), 2.44-2.25 (m, 2H), 2.22-1.77 (m, 10H), 1.75-1.48 (m, 6H), 1.34-1.22 (m, 3H), 0.93-0.84 (m, 3H), 0.67-0.46 (m, 2H) |
| 404 | 2-ethylbutyl (((7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 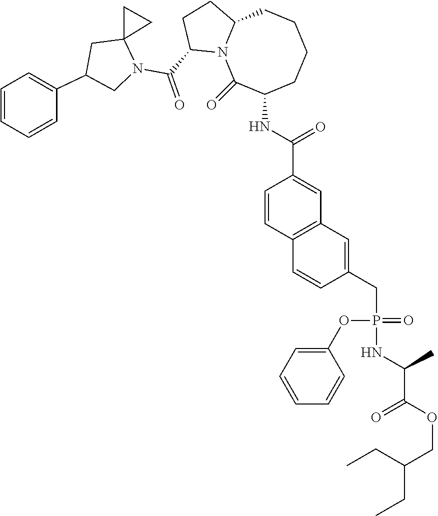 [SMILES: CCC(CC)COC(=O)[C@H](C)NP(=O)(Cc1ccc2ccc(C(=O)N[C@H]3CCCC[C@H]4CC[C@@H](C(=O)N5CC(c6ccccc6)CC56CC6)N4C3=O)cc2c1)Oc1ccccc1] | 861.6 | ¹H NMR (400 MHz, DMSO) δ 8.74-8.55 (m, 1H), 8.46 (d, J = 5.0 Hz, 1H), 8.00- 7.81 (m, 4H), 7.61 (d, J = 8.3 Hz, 1H), 7.36-7.28 (m, 5H), 7.28-7.18 (m, 2H), 7.18-7.08 (m, 3H), 5.77-5.52 (m, 1H), 5.11-4.91 (m, 1H), 4.59-4.09 (m, 3H), 3.91-3.82 (m, 2H), 3.80-3.72 (m, 1H), 3.62-3.55 (m, 1H), 3.55-3.50 (m, 1H), 3.49-3.43 (m, 1H), 2.35-2.15 (m, 2H), 2.14-1.97 (m, 3H), 1.97-1.81 (m, 5H), 1.81-1.71 (m, 2H), 1.70-1.51 (m, 4H), 1.51-1.30 (m, 2H), 1.28-1.20 (m, 3H), 1.05 (2d, J = 7.0 Hz, 3H), 0.83-0.69 (m, 6H), 0.54-0.39 (m, 2H) |
| 405 | butyl (((7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 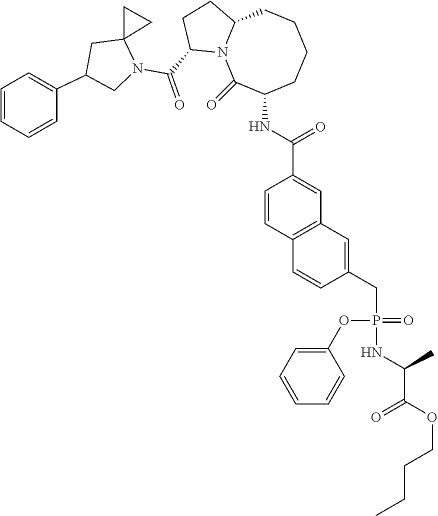 [SMILES: CCCCOC(=O)[C@H](C)NP(=O)(Cc1ccc2ccc(C(=O)N[C@H]3CCCC[C@H]4CC[C@@H](C(=O)N5CC(c6ccccc6)CC56CC6)N4C3=O)cc2c1)Oc1ccccc1] | 833.5 | ¹H NMR (400 MHz, CDCl₃) δ 8.33-8.26 (m, 1H), 7.93-7.80 (m, 4H), 7.64-7.55 (m, 1H), 7.47-7.36 (m, 1H), 7.35-7.26 (m, 6H), 7.24-7.02 (m, 4H), 5.26-5.13 (m, 1H), 4.60-3.45 (m, 10H), 3.37-3.04 (m, 1H), 2.55-2.37 (m, 1H), 2.25-2.18 (m, 1H), 2.13-1.42 (m, 16H), 1.36-1.10 (m, 5H), 0.95-0.76 (m, 3H), 0.63-0.47 (m, 2H) |
| 406 | butyl ((difluoro(7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl) (naphthalen-1- yloxy)phosphoryl)- L-alaninate | 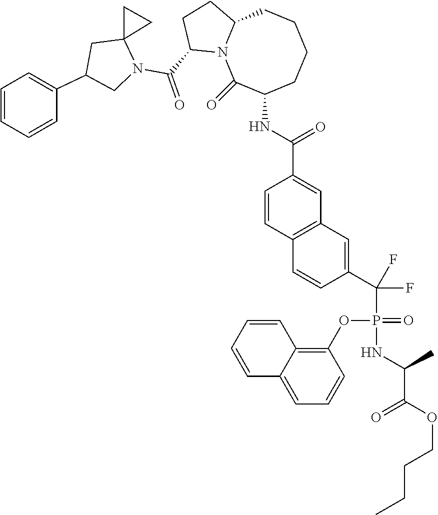 [SMILES: CCCCOC(=O)[C@H](C)NP(=O)(Oc1cccc2ccccc12)C(F)(F)c1ccc2ccc(C(=O)N[C@H]3CCCC[C@H]4CC[C@@H](C(=O)N5CC(c6ccccc6)CC56CC6)N4C3=O)cc2c1] | 919.0 | ¹H NMR (400 MHz, CDCl₃) δ 8.37-8.27 (m, 1H), 8.26-8.19 (m, 1H), 8.00-7.83 (m, 5H), 7.83-7.75 (m, 1H), 7.66-7.54 (m, 1H), 7.54-7.37 (m, 4H), 7.36-7.27 (m, 4H), 7.25-7.21 (m, 1H), 5.27-5.11 (m, 1H), 4.64-4.15 (m, 3H), 4.14-3.85 (m, 4H), 3.69-3.08 (m, 2H), 2.54-2.36 (m, 1H), 2.27-1.86 (m, 9H), 1.80-1.61 (m, 5H), 1.53-1.42 (m, 3H), 1.32-1.24 (m, 5H), 0.91-0.84 (m, 3H), 0.63-0.45 (m, 2H) |
| 407 | butyl ((difluoro(7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 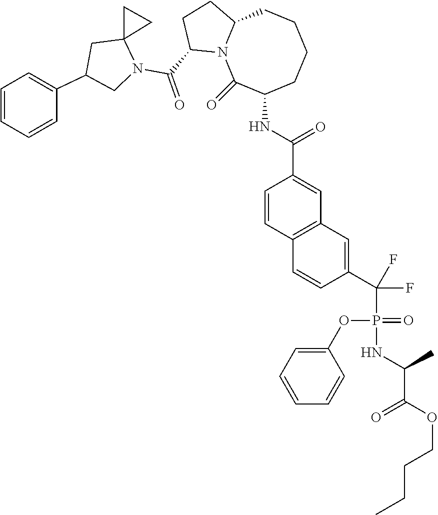 [SMILES: CCCCOC(=O)[C@H](C)NP(=O)(Oc1ccccc1)C(F)(F)c1ccc2ccc(C(=O)N[C@H]3CCCC[C@H]4CC[C@@H](C(=O)N5CC(c6ccccc6)CC56CC6)N4C3=O)cc2c1] | 869.3 | ¹H NMR (400 MHz, DMSO) δ 8.71-8.60 (m, 2H), 8.32-8.17 (m, 1H), 8.15-8.00 (m, 3H), 7.82-7.68 (m, 1H), 7.40-7.11 (m, 9H), 6.89-6.75 (m, 1H), 5.06-4.95 (m, 1H), 4.59-3.43 (m, 9H), 2.36-2.15 (m, 2H), 2.13-1.71 (m, 10H), 1.60-1.37 (m, 6H), 1.23-1.14 (m, 4H), 0.89-0.74 (m, 4H), 0.55-0.38 (m, 2H) |
| 408 | S,S′-(((((7- (((3S,6S,10aS)-3- ((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphoryl)bis(oxy)) bis(ethane-2,1- diyl)) bis(2- (oxetan-3- yl)ethanethioate) Or S,S′-(((((7- (((3S,6S,10aS)-3- (3S,4R)-3-cyano-4- (2-oxo-1,2- dihydropyridin-4- yl)pyrrolidine-1- carbonyl)-5- oxodecahydropyrrolo [1,2-a]azocin-6- yl)carbamoyl) naphthalen-2- | 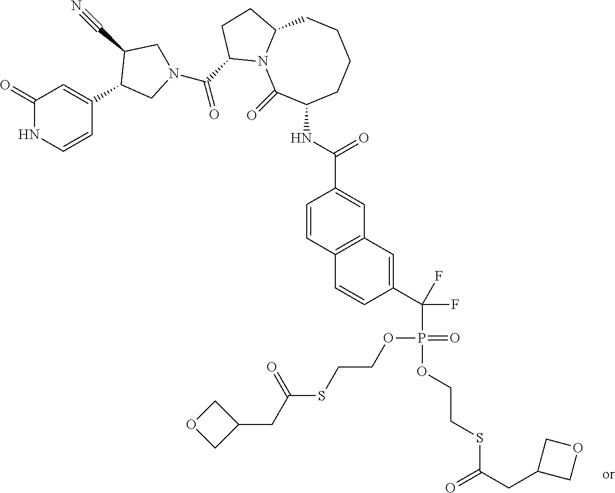 [SMILES: N#C[C@H]1CN(C(=O)[C@@H]2CC[C@@H]3CCCC[C@H](NC(=O)c4ccc5ccc(C(F)(F)P(=O)(OCCSC(=O)CC6COC6)OCCSC(=O)CC6COC6)cc5c4)C(=O)N32)C[C@@H]1c1cc[nH]c(=O)c1] | 998.2 | ¹H NMR (400 MHz, DMSO) δ 11.61 (s, 1H), 8.75-8.57 (m, 2H), 8.39-8.25 (m, 1H), 8.24-8.02 (m, 3H), 7.83-7.62 (m, 1H), 7.38 (d, J = 6.7 Hz, 1H), 6.42-6.32 (m, 1H), 6.30-6.18 (m, 1H), 5.09-4.91 (m, 1H), 4.72-4.07 (m, 14H), 3.99-3.46 (m, 6H), 3.23-3.06 (m, 6H), 3.02-2.64 (m, 4H), 2.34-2.19 (m, 1H), 2.06-1.39 (m, 11H) (free base) |
| | yl)difluoromethyl) phosphoryl)bis(oxy)) bis(ethane-2,1- diyl)) bis(2- (oxetan-3- yl)ethanethioate) | 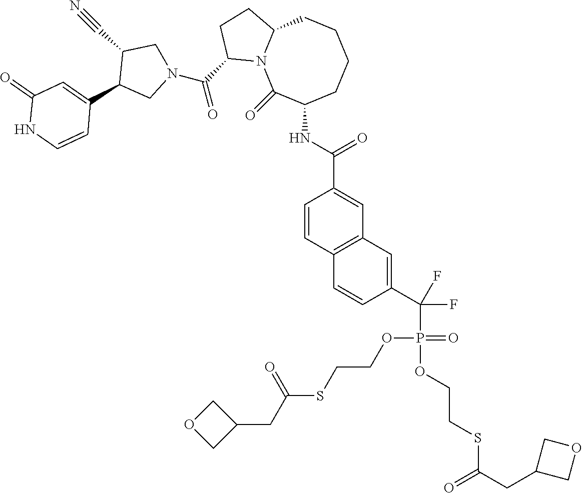 [SMILES: N#C[C@@H]1CN(C(=O)[C@@H]2CC[C@@H]3CCCC[C@H](NC(=O)c4ccc5ccc(C(F)(F)P(=O)(OCCSC(=O)CC6COC6)OCCSC(=O)CC6COC6)cc5c4)C(=O)N32)C[C@H]1c1cc[nH]c(=O)c1] Pyrrolidine building block SFC peak 1 was used for synthesis and biological testing | | |
| 409 | propyl (((7- (((3S,6S,10aS)-5- oxo-3-((R)-6-(2- oxo-1,2- dihydropyridin-4- yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 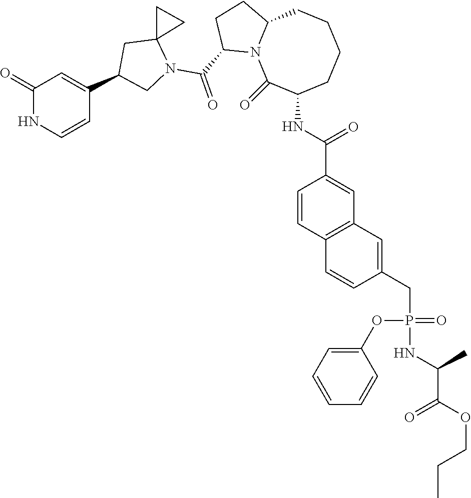 [SMILES: CCCOC(=O)[C@H](C)NP(=O)(Cc1ccc2ccc(C(=O)N[C@H]3CCCC[C@H]4CC[C@@H](C(=O)N5C[C@@H](c6cc[nH]c(=O)c6)CC56CC6)N4C3=O)cc2c1)Oc1ccccc1] | 836.4 | ¹H NMR (400 MHz, DMSO) δ 11.43 (s, 1H), 8.68-8.58 (m, 1H), 8.44 (s, 1H), 8.01-7.85 (m, 4H), 7.60 (d, J = 8.4 Hz, 1H), 7.36-7.20 (m, 3H), 7.18-7.07 (m, 3H), 6.26 (s, 1H), 6.18 (d, J = 6.8 Hz, 1H), 5.76-5.57 (m, 1H), 5.07-4.93 (m, 1H), 4.56-4.43 (m, 1H), 4.34-4.18 (m, 1H), 4.12-4.00 (m, 1H), 3.93-3.74 (m, 3H), 3.74-3.65 (m, 1H), 3.59-3.44 (m, 3H), 2.34-2.21 (m, 1H), 2.18-1.65 (m, 11H), 1.65-1.34 (m, 6H), 1.07 (d, J = 7.1 Hz, 1H), 0.99 (d, J = 7.1 Hz, 2H), 0.83- 0.75 (m, 3H), 0.54 0.39 (m, 2H) |
| 410 | isobutyl (((7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 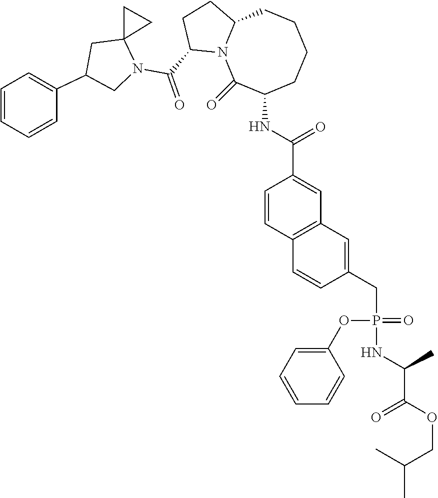 [SMILES: CC(C)COC(=O)[C@H](C)NP(=O)(Cc1ccc2ccc(C(=O)N[C@H]3CCCC[C@H]4CC[C@@H](C(=O)N5CC(c6ccccc6)CC56CC6)N4C3=O)cc2c1)Oc1ccccc1] | 833.6 | ¹H NMR (400 MHz, DMSO) δ 8.69-8.54 (m, 1H), 8.52-8.39 (m, 1H), 8.02-7.83 (m, 4H), 7.66-7.55 (m, 1H), 7.39-7.04 (m, 10H), 5.80-5.51 (m, 1H), 5.11-4.90 (m, 1H), 4.65-4.02 (m, 3H), 3.93-3.68 (m, 3H), 3.58-3.46 (m, 3H), 2.35-2.17 (m, 2H), 2.13-1.49 (m, 15H), 1.04 (2d, J = 7.0 Hz, 3H), 0.82- 0.79 (m, 6H), 0.55- 0.38 (m, 2H) |
| 411 | propyl ((fluoro(7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 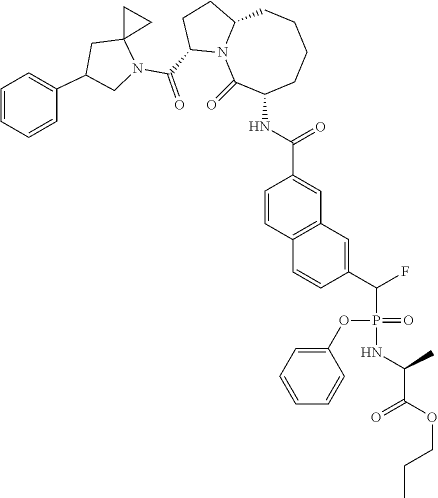 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5CC(c6ccccc6)CC56CC6)N4C3=O)cc2c1] | 837.5 | ¹H NMR (400 MHz, DMSO) δ 8.73-8.61 (m, 1H), 8.60-8.43 (m, 1H), 8.13 (s, 1H), 8.07-7.81 (m, 3H), 7.79-7.58 (m, 1H), 7.42-7.07 (m, 9H), 6.67-6.09 (m, 2H), 5.36-4.98 (m, 1H), 4.58-4.07 (m, 3H), 4.02-3.64 (m, 4H), 3.61-3.49 (m, 2H), 2.37-2.16 (m, 2H), 2.11-1.69 (m, 10H), 1.66-1.33 (m, 6H), 1.18-1.06 (m, 2H), 1.02-0.68 (m, 4H), 0.60-0.33 (m, 2H) |
| 412 | propyl ((fluoro(7- (((3S,6S,10aS)-5- oxo-3-((S)-6-(2- oxo-1,2- dihydropyridin-4- yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 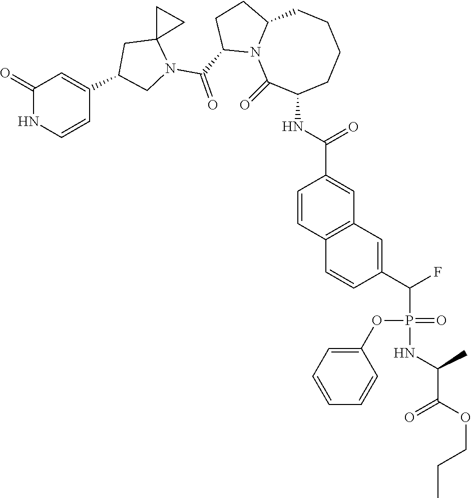 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5C[C@H](c6cc[nH]c(=O)c6)CC56CC6)N4C3=O)cc2c1] | 854.5 | ¹H NMR (400 MHz, CDCl₃) δ 9.80-7.27 (m, 12H), 7.24-6.56 (m, 3H), 6.24-5.73 (m, 2H), 5.39-5.16 (m, 1H), 4.82-4.46 (m, 2H), 4.43-4.27 (m, 2H), 4.19-3.80 (m, 3H), 3.77-3.59 (m, 1H), 3.51-3.28 (m, 1H), 2.45-2.35 (m, 1H), 2.28-1.46 (m, 15H), 1.45-1.02 (m, 6H), 1.01-0.78 (m, 3H), 0.60-0.40 (m, 1H) |
| 413 | propyl ((fluoro(7- (((3S,6S,10aS)-5- oxo-3-((R)-6-(2- oxo-1,2- dihydropyridin-4- yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 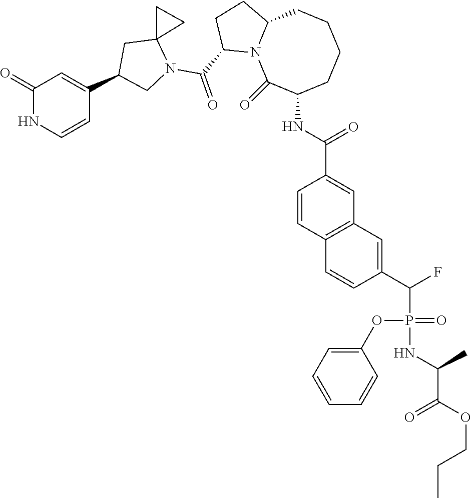 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5C[C@@H](c6cc[nH]c(=O)c6)CC56CC6)N4C3=O)cc2c1] | 854.4 | ¹H NMR (400 MHz, CDCl₃) δ 8.94-7.29 (m, 12H), 7.25-6.99 (m, 3H), 6.19-5.87 (m, 1H), 5.38-5.02 (m, 1H), 4.72-4.13 (m, 5H), 4.11-3.99 (m, 2H), 3.95-3.81 (m, 1H), 3.72-3.54 (m, 1H), 2.42-1.92 (m, 9H), 1.89-1.45 (m, 7H), 1.42-1.09 (m, 6H), 0.95-0.77 (m, 3H), 0.56-0.41 (m, 1H) (TFA salt) |
| 414 | propyl ((difluoro(7- (((3S,6S,10aS)-5- oxo-3-((R)-6-(2- oxo-1,2- dihydropyridin-4- yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 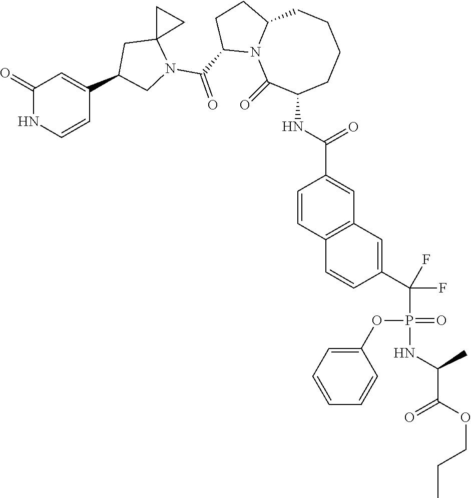 [SMILES: CCCOC(=O)[C@H](C)NP(=O)(Oc1ccccc1)C(F)(F)c1ccc2ccc(C(=O)N[C@H]3CCCC[C@H]4CC[C@@H](C(=O)N5C[C@@H](c6cc[nH]c(=O)c6)CC56CC6)N4C3=O)cc2c1] | 872.3 | ¹H NMR (400 MHz, DMSO) δ 11.42 (s, 1H), 8.73-8.49 (m, 2H), 8.48-8.25 (m, 1H), 8.23-8.03 (m, 3H), 7.84-7.71 (m, 1H), 7.44-7.31 (m, 2H), 7.30-7.24 (m, 1H), 7.23-7.10 (m, 3H), 6.90-6.77 (m, 1H), 6.40-6.22 (m, 1H), 6.21-6.07 (m, 1H), 5.34-4.95 (m, 1H), 4.57-4.43 (m, 1H), 4.36-3.74 (m, 6H), 3.73-3.63 (m, 1H), 2.36-2.23 (m, 1H), 2.13-1.95 (m, 5H), 1.91-1.80 (m, 3H), 1.79-1.68 (m, 2H), 1.66-1.39 (m, 6H), 1.26-1.16 (m, 4H), 0.87-0.76 (m, 3H), 0.59-0.39 (m, 2H) |
| 415 | propyl ((fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 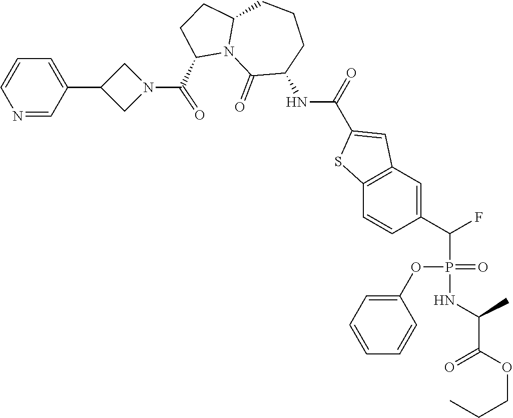 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cccnc6)C5)N4C3=O)cc2c1] | 790.3 | ¹H NMR (400 MHz, CDCl₃) δ 9.10-8.71 (m, 2H), 8.60-8.16 (m, 1H), 8.04-7.28 (m, 7H), 7.26-7.17 (m, 2H), 7.16-7.02 (m, 2H), 6.08-5.99 (m, 1H), 5.22-4.40 (m, 5H), 4.31-3.59 (m, 7H), 2.37-1.51 (m, 12H), 1.38-1.20 (m, 3H), 0.97-0.78 (m, 3H) (TFA salt) |
| 416 | butyl ((naphthalen- 1-yloxy)((7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)phosphoryl)- L-alaninate | 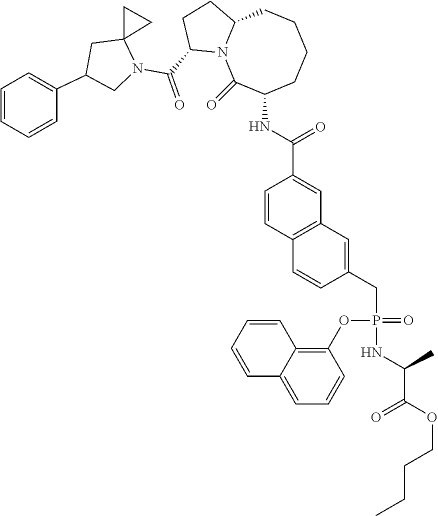 [SMILES: CCCCOC(=O)[C@H](C)NP(=O)(Cc1ccc2ccc(C(=O)N[C@H]3CCCC[C@H]4CC[C@@H](C(=O)N5CC(c6ccccc6)CC56CC6)N4C3=O)cc2c1)Oc1cccc2ccccc12] | 883.4 | ¹H NMR (400 MHz, DMSO) δ 8.69-8.58 (m, 1H), 8.49-8.42 (m, 1H), 8.05-7.86 (m, 6H), 7.71-7.63 (m, 2H), 7.55-7.18 (m, 9H), 5.92-5.69 (m, 1H), 5.11-4.93 (m, 1H), 4.59-4.08 (m, 3H), 4.01-3.44 (m, 7H), 2.36-2.16 (m, 2H), 2.14-1.67 (m, 10H), 1.66-1.45 (m, 4H), 1.42-1.30 (m, 2H), 1.26-1.12 (m, 2H), 1.11-0.95 (m, 3H), 0.83-0.73 (m, 3H), 0.55-0.40 (m, 2H) |
| 417 | isobutyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 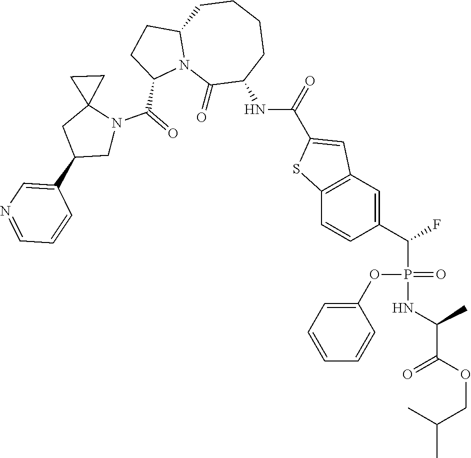 [SMILES: CC(C)COC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | 858.3 | ¹H NMR (400 MHz, CDCl₃) δ 8.63-8.42 (m, 2H), 8.01-7.73 (m, 3H), 7.73-7.47 (m, 3H), 7.46-7.28 (m, 2H), 7.26-7.16 (m, 2H), 7.15-7.02 (m, 2H), 6.11-5.80 (m, 1H), 5.26-5.10 (m, 1H), 4.64-4.46 (m, 1H), 4.39-4.23 (m, 1H), 4.20-3.69 (m, 6H), 3.65-3.49 (m, 1H), 2.45-1.94 (m, 12H), 1.90-1.63 (m, 5H), 1.38-1.09 (m, 3H), 1.02-0.82 (m, 6H), 0.63-0.44 (m, 2H) |
| 418 | cyclopropyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 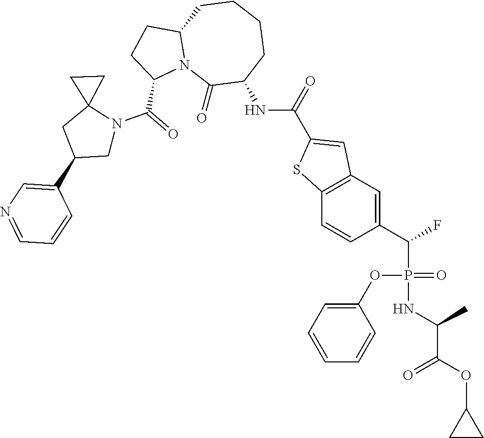 [SMILES: C[C@H](NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1)C(=O)OC1CC1] | 842.5 | ¹H NMR (400 MHz, DMSO) δ 8.90-8.81 (m, 1H), 8.66 (s, 1H), 8.50 (d, J = 4.4 Hz, 1H), 8.30 (s, 1H), 8.14-8.03 (m, 2H), 7.94 (d, J = 7.9 Hz, 1H), 7.61 (d, J = 8.3 Hz, 1H), 7.50-7.42 (m, 1H), 7.41-7.32 (m, 2H), 7.24-7.11 (m, 3H), 6.28-6.08 (m, 2H), 5.00-4.89 (m, 1H), 4.56-4.47 (m, 1H), 4.30-4.20 (m, 1H), 4.19-4.11 (m, 1H), 4.06-3.91 (m, 1H), 3.88-3.64 (m, 3H), 2.27-1.52 (m, 16H), 1.16-0.88 (m, 3H), 0.73-0.37 (m, 6H) |
| 419 | propyl ((difluoro(7- (((3S,6S,10aS)-5- oxo-3-((S)-6-(2- oxo-1,2- dihydropyridin-4- yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 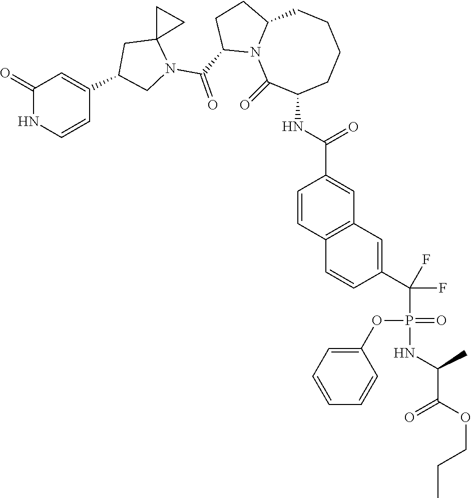 [SMILES: CCCOC(=O)[C@H](C)NP(=O)(Oc1ccccc1)C(F)(F)c1ccc2ccc(C(=O)N[C@H]3CCCC[C@H]4CC[C@@H](C(=O)N5C[C@H](c6cc[nH]c(=O)c6)CC56CC6)N4C3=O)cc2c1] | 872.4 | ¹H NMR (400 MHz, DMSO) δ 11.49- 11.31 (m, 1H), 8.70- 8.62 (m, 2H), 8.32- 8.26 (m, 1H), 8.17- 8.10 (m, 1H), 8.10- 8.04 (m, 2H), 7.83- 7.74 (m, 1H), 7.41- 7.32 (m, 2H), 7.31- 7.24 (m, 1H), 7.23- 7.16 (m, 2H), 7.15- 7.10 (m, 1H), 6.89- 6.78 (m, 1H), 6.27- 6.21 (m, 1H), 6.20- 6.13 (m, 1H), 5.06- 4.95 (m, 1H), 4.58- 4.49 (m, 1H), 4.35- 4.15 (m, 2H), 3.95- 3.79 (m, 3H), 3.65- 3.51 (m, 1H), 3.25- 3.20 (m, 1H), 2.20- 2.17 (m, 2H), 2.07- 2.00 (m, 2H), 1.96- 1.71 (m, 8H), 1.58- 1.40 (m, 5H), 1.25- 1.15 (m, 4H), 0.85- 0.76 (m, 3H), 0.53- 0.41 (m, 2H) |
| 420 | dibutyl 2,2′-((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) bis(azanediyl)) (2S,2′S)-dipropionate | 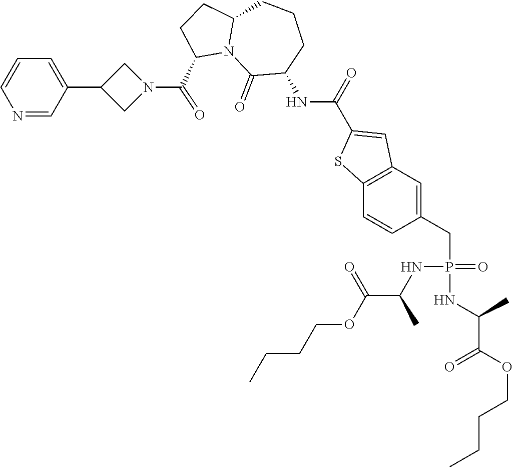 [SMILES: CCCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)N[C@@H](C)C(=O)OCCCC] | 837.4 | ¹H NMR (400 MHz, CDCl₃) δ 8.84-8.55 (m, 2H), 8.12-7.73 (m, 5H), 7.69-7.37 (m, 3H), 5.10-4.58 (m, 4H), 4.56-4.42 (m, 1H), 4.24-4.18 (m, 1H), 4.12-3.91 (m, 8H), 3.35-3.22 (m, 2H), 3.17-3.06 (m, 1H), 3.05-2.90 (m, 1H), 2.34-2.20 (m, 2H), 2.12-2.02 (m, 4H), 1.99-1.91 (m, 2H), 1.87-1.73 (m, 2H), 1.63-1.53 (m, 4H), 1.43-1.29 (m, 10H), 0.93 (t, J = 7.3 Hz, 6H) |
| 421 | propyl (((7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 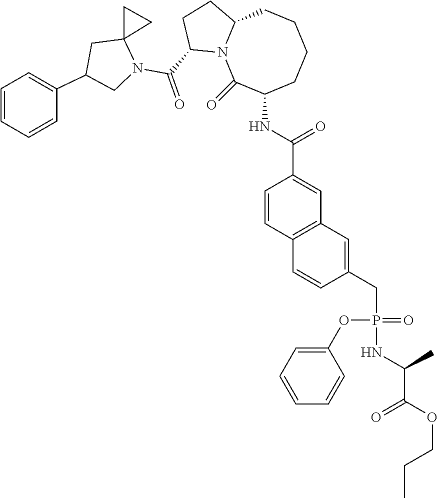 [SMILES: CCCOC(=O)[C@H](C)NP(=O)(Cc1ccc2ccc(C(=O)N[C@H]3CCCC[C@H]4CC[C@@H](C(=O)N5CC(c6ccccc6)CC56CC6)N4C3=O)cc2c1)Oc1ccccc1] | 819.5 | ¹H NMR (400 MHz, DMSO) δ 8.71-8.56 (m, 1H), 8.47-8.40 (m, 1H), 7.99-7.86 (m, 4H), 7.63-7.56 (m, 1H), 7.37-7.26 (m, 6H), 7.25-7.17 (m, 1H), 7.17-7.08 (m, 3H), 5.76-5.55 (m, 1H), 5.07-4.91 (m, 1H), 4.59-4.07 (m, 3H), 3.94-3.41 (m, 7H), 2.37-2.15 (m, 2H), 2.12-1.68 (m, 10H), 1.66-1.37 (m, 6H), 1.03 (2d, J = 7.1 Hz, 3H), 0.84- 0.72 (m, 3H), 0.54- 0.40 (m, 2H) |
| 422 | ethyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 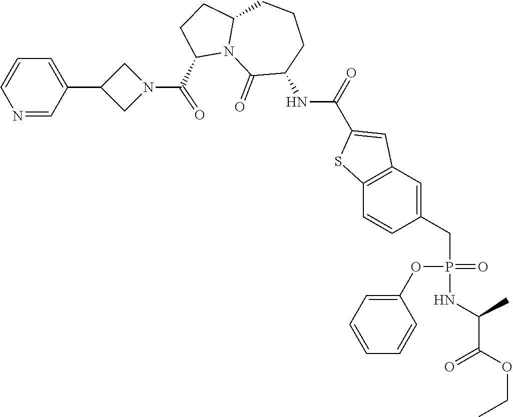 [SMILES: CCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 758.1 | ¹H NMR (400 MHz, CDCl₃) δ 9.20-8.11 (m, 4H), 8.00-7.29 (m, 5H), 7.14-6.99 (m, 2H), 5.08-3.78 (m, 10H), 3.53-2.98 (m, 3H), 2.26-2.01 (m, 4H), 1.96-1.68 (m, 6H), 1.22-1.01 (m, 6H) |
| 423 | isopropyl (((7- (((3S,6S,10aS)-5- oxo-3-((S)-6-(2- oxo-1,2- dihydropyridin-4- yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 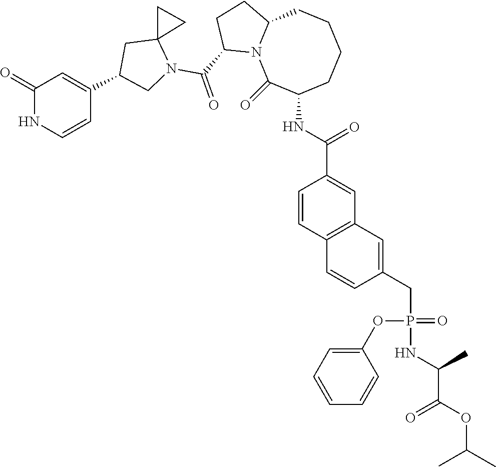 [SMILES: CC(C)OC(=O)[C@H](C)NP(=O)(Cc1ccc2ccc(C(=O)N[C@H]3CCCC[C@H]4CC[C@@H](C(=O)N5C[C@H](c6cc[nH]c(=O)c6)CC56CC6)N4C3=O)cc2c1)Oc1ccccc1] | 836.5 | ¹H NMR (400 MHz, CDCl₃) δ 8.36-8.26 (m, 1H), 7.98-7.71 (m, 5H), 7.57-7.49 (m, 1H), 7.42-7.35 (m, 1H), 7.32-7.27 (m, 2H), 7.18-7.10 (m, 3H), 6.66-6.45 (m, 2H), 5.36-5.22 (m, 1H), 4.98-4.86 (m, 1H), 4.64-4.31 (m, 3H), 4.00-3.86 (m, 1H), 3.59-3.42 (m, 4H), 2.25-1.64 (m, 16H), 1.27-1.15 (m, 9H), 0.59-0.44 (m, 2H) |
| 424 | isopropyl (((7- (((3S,6S,10aS)-5- oxo-3-((R)-6-(2- oxo-1,2- dihydropyridin-4- yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 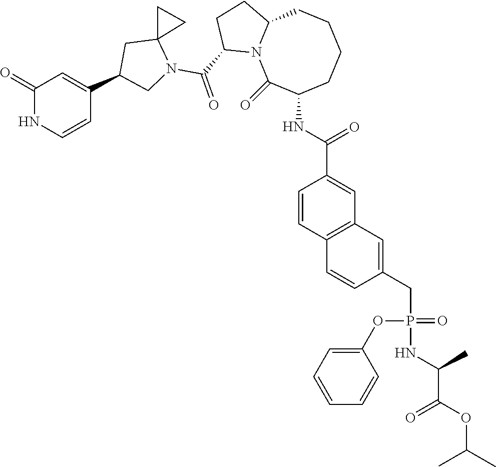 [SMILES: CC(C)OC(=O)[C@H](C)NP(=O)(Cc1ccc2ccc(C(=O)N[C@H]3CCCC[C@H]4CC[C@@H](C(=O)N5C[C@@H](c6cc[nH]c(=O)c6)CC56CC6)N4C3=O)cc2c1)Oc1ccccc1] | 836.5 | ¹H NMR (400 MHz, CDCl₃) δ 8.61-7.27 (m, 8H), 7.23-6.84 (m, 4H), 6.46-5.90 (m, 2H), 5.32-5.12 (m, 1H), 5.03-4.81 (m, 1H), 4.55-4.47 (m, 1H), 4.39-4.26 (m, 1H), 4.11-3.78 (m, 3H), 3.65-3.46 (m, 2H), 3.35-3.21 (m, 1H), 2.43-2.28 (m, 1H), 2.04-1.79 (m, 6H), 1.76-1.57 (m, 4H), 1.45-1.30 (m, 6H), 1.22-1.16 (m, 6H), 1.14-1.08 (m, 2H), 0.60-0.42 (m, 2H) |
| 425 | propyl ((1-(7- (((3S,6S,10aS)-5- oxo-3-((R)-6- phenyl-4- azaspiro[2.4]heptane- 4- carbonyl)decahydro pyrrolo[1,2- a]azocin-6- yl)carbamoyl) naphthalen-2- yl)ethyl)(phenoxy) phosphoryl)-L- alaninate Or propyl ((1-(7- (((3S,6S,10aS)-5- oxo-3-((S)-6- phenyl-4- azaspiro[2.4]heptane- 4- carbonyl)decahydro pyrrolo[1,2- a]azocin-6- yl)carbamoyl) naphthalen-2- yl)ethyl)(phenoxy) phosphoryl)-L- alaninate | 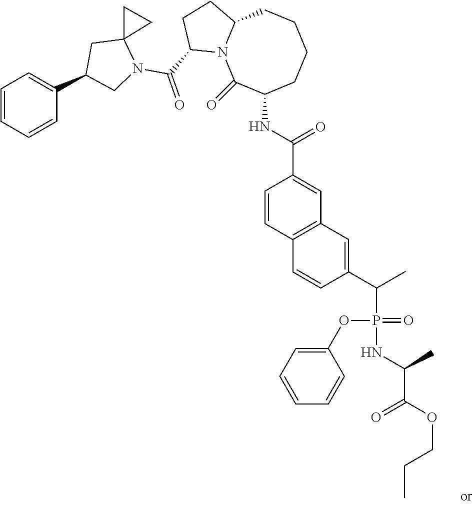 [SMILES: CCCOC(=O)[C@H](C)NP(=O)(Oc1ccccc1)C(C)c1ccc2ccc(C(=O)N[C@H]3CCCC[C@H]4CC[C@@H](C(=O)N5C[C@@H](c6ccccc6)CC56CC6)N4C3=O)cc2c1] | 833.7 | ¹H NMR (400 MHz, DMSO) δ 8.58 (d, J = 7.3 Hz, 1H), 8.47 (s, 1H), 7.94-7.82 (m, 4H), 7.58 (d, J = 8.6 Hz, 1H), 7.37-7.26 (m, 4H), 7.24-7.18 (m, 1H), 5.08-4.90 (m, 1H), 4.49 (t, J = 8.2 Hz, 1H), 4.33- 4.21 (m, 1H), 4.12 (t, J = 8.7 Hz, 1H), 3.73 (t, J = 9.9 Hz, 1H), 3.58-3.48 (m, 1H), 3.23-3.15 (m, 1H), 2.36-2.17 (m, 2H), 2.15-2.08 (m, 1H), 2.06-1.94 (m, 3H), 1.94-1.69 (m, 6H), 1.67-1.59 (m, 1H), 1.58-1.45 (m, 6H), 0.56-0.39 (m, 2H) |
| | | 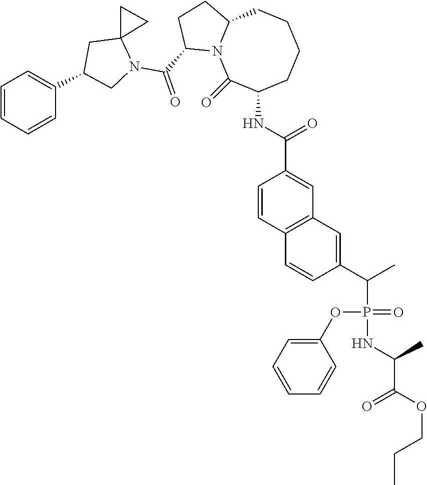 [SMILES: CCCOC(=O)[C@H](C)NP(=O)(Oc1ccccc1)C(C)c1ccc2ccc(C(=O)N[C@H]3CCCC[C@H]4CC[C@@H](C(=O)N5C[C@H](c6ccccc6)CC56CC6)N4C3=O)cc2c1] Pyrrolidine building block SFC peak 1 was used for synthesis and biological testing | | |
| 426 | propyl (morpholino((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl)- L-alaninate | 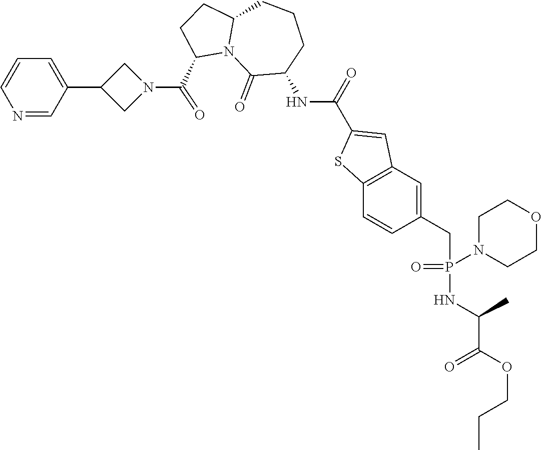 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)N1CCOCC1] | 765.4 | ¹H NMR (400 MHz, DMSO) δ 8.83-8.71 (m, 1H), 8.59-8.52 (m, 1H), 8.50-8.41 (m, 1H), 8.22-8.12 (m, 1H), 7.95-7.82 (m, 3H), 7.45-7.30 (m, 2H), 5.28-5.05 (m, 1H), 4.84-4.56 (m, 2H), 4.49-4.18 (m, 3H), 4.07-3.72 (m, 6H), 3.45-3.35 (m, 4H), 3.26-3.15 (m, 2H), 2.98-2.74 (m, 4H), 2.26-2.13 (m, 1H), 2.08-1.92 (m, 2H), 1.90-1.66 (m, 7H), 1.64-1.51 (m, 2H), 1.32-1.21 (m, 3H), 0.95-0.81 (m, 3H) |
| 427 | propyl ((fluoro(7- (((3S,6S,10aS)-5- oxo-3-(3-(pyridin- 3-yl)pyrrolidine-1- carbonyl)decahydro pyrrolo[1,2- a]azocin-6- yl)carbamoyl) naphthalen-2- yl)methyl)(phenoxy) phosphoryl)-L- alaninate | 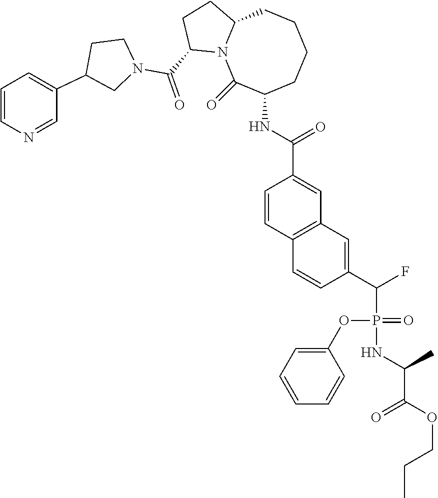 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5CCC(c6cccnc6)C5)N4C3=O)cc2c1] | 812.4 | ¹H NMR (400 MHz, DMSO) δ 8.61-8.44 (m, 2H), 8.38-8.27 (m, 1H), 7.95-7.78 (m, 4H), 7.68-7.37 (m, 3H), 7.32-7.28 (m, 1H), 7.27-7.24 (m, 2H), 7.20-7.07 (m, 3H), 5.26-4.93 (m, 1H), 4.69-4.51 (m, 1H), 4.45-4.14 (m, 2H), 4.08-3.63 (m, 5H), 3.60-3.15 (m, 5H), 2.49-2.29 (m, 2H), 2.18-2.00 (m, 6H), 1.88-1.63 (m, 6H), 1.60-1.46 (m, 2H), 1.20-1.08 (m, 3H), 0.92-0.78 (m, 3H) |
| 428 | propyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(2,2,2- trifluoroethoxy) phosphoryl)-L- alaninate | 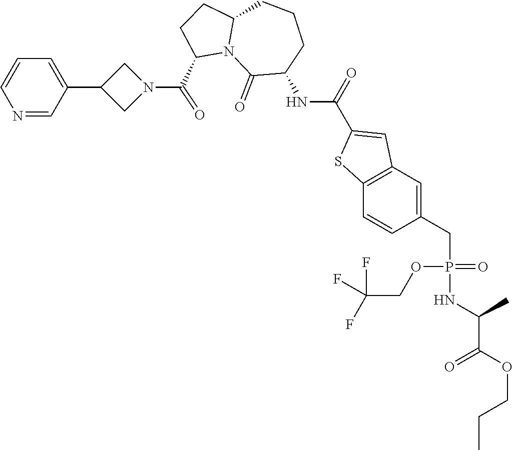 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)OCC(F)(F)F] | 776.2 [M − H]⁻ | ¹H NMR (400 MHz, DMSO) δ 8.83-8.53 (m, 3H), 8.22-8.09 (m, 2H), 7.97-7.90 (m, 1H), 7.87-7.81 (m, 1H), 7.69-7.50 (m, 1H), 7.42-7.34 (m, 1H), 5.62-5.48 (m, 1H), 4.84-4.61 (m, 2H), 4.48-4.26 (m, 5H), 4.06-3.83 (m, 5H), 3.80-3.65 (m, 1H), 3.43-3.29 (m, 2H), 2.26-2.16 (m, 1H), 2.08-1.92 (m, 2H), 1.91-1.64 (m, 7H), 1.62-1.48 (m, 2H), 1.30-1.08 (m, 3H), 0.91-0.80 (m, 3H) |
| 429 | propyl ((fluoro(7- (((3S,6S,10aS)-5- oxo-3-(6-(pyridin- 3-yl)-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 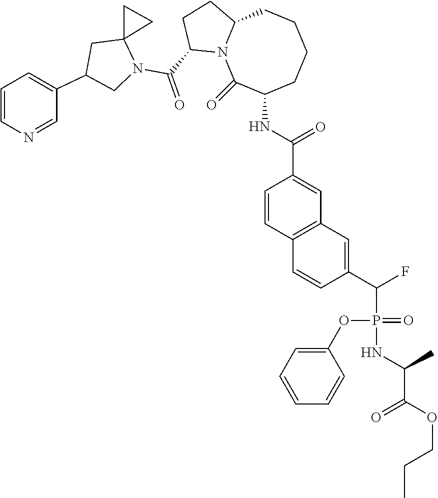 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5CC(c6cccnc6)CC56CC6)N4C3=O)cc2c1] | 838.5 | ¹H NMR (400 MHz, DMSO) δ 8.72-8.64 (m, 1H), 8.64-8.50 (m, 2H), 8.49-8.38 (m, 1H), 8.23-7.98 (m, 4H), 7.83-7.69 (m, 2H), 7.38-7.28 (m, 3H), 7.23-7.09 (m, 3H), 6.38-6.11 (m, 2H), 5.09-4.95 (m, 1H), 4.75-4.06 (m, 3H), 4.00-3.46 (m, 5H), 2.42-1.36 (m, 18H), 1.21-0.95 (m, 3H), 0.83-0.72 (m, 3H), 0.58-0.34 (m, 2H) |
| 430 | propyl O-methyl-N- (((2-(((3S,6S,9aS)- 5-oxo-3-(3- (pyridin-3- yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- serinate | 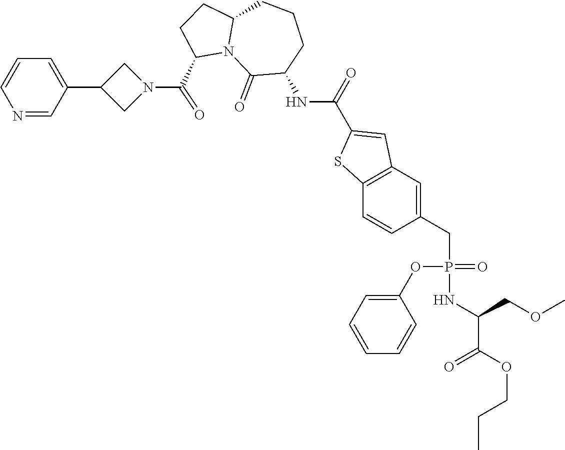 [SMILES: CCCOC(=O)[C@H](COC)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 802.5 | ¹H NMR (400 MHz, CDCl₃) δ 8.64-8.53 (m, 2H), 7.96-7.61 (m, 5H), 7.54-7.31 (m, 2H), 7.29 (d, J = 7.6 Hz, 1H), 7.26 (s, 1H), 7.20-7.07 (m, 3H), 5.06-4.37 (m, 5H), 4.24-4.15 (m, 1H), 4.13-3.92 (m, 5H), 3.65-3.41 (m, 4H), 3.22-3.05 (m, 4H), 2.31-2.17 (m, 2H), 2.11-1.99 (m, 4H), 1.95 (t, J = 10.1 Hz, 2H), 1.85-1.69 (m, 2H), 1.66-1.50 (m, 2H), 0.95-0.82 (m, 3H) |
| 431 | propyl O-methyl-N- (((2-(((3S,6S,9aS)- 5-oxo-3-(3- (pyridin-3- yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- homoserinate | 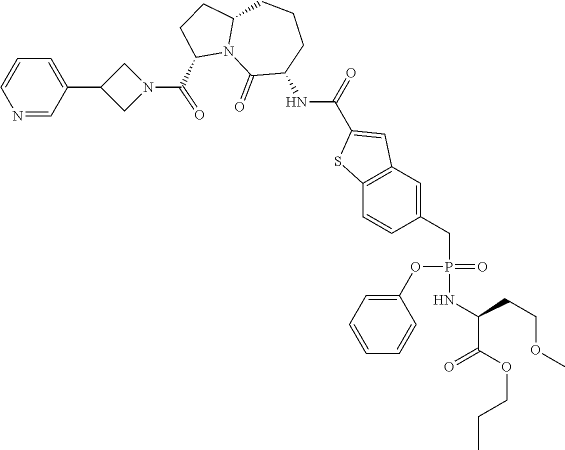 [SMILES: CCCOC(=O)[C@H](CCOC)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 816.4 | ¹H NMR (400 MHz, DMSO) δ 8.82-8.72 (m, 1H), 8.59-8.52 (m, 1H), 8.50-8.40 (m, 1H), 8.22-8.19 (m, 1H), 7.97-7.83 (m, 3H), 7.48-7.28 (m, 4H), 7.17-7.07 (m, 3H), 5.73-5.47 (m, 1H), 4.84-4.57 (m, 2H), 4.48-4.19 (m, 3H), 4.05-3.77 (m, 6H), 3.53-3.35 (m, 2H), 3.14-2.89 (m, 5H), 2.28-2.15 (m, 1H), 2.08-1.93 (m, 2H), 1.90-1.45 (m, 11H), 0.81 (t, J = 14.0, 6.7 Hz, 3H) |
| 432 | propyl (2S)-2-((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino)- 3-(pyridin-2- yl)propanoate | 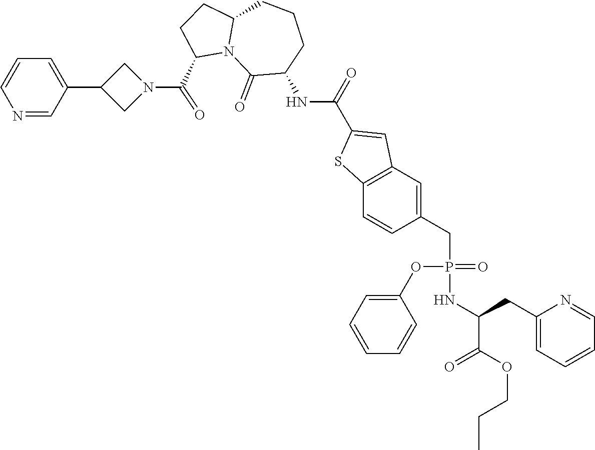 [SMILES: CCCOC(=O)[C@H](Cc1ccccn1)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 849.3 | ¹H NMR (400 MHz, CDCl₃) δ 8.71-8.49 (m, 2H), 8.23-8.10 (m, 1H), 8.04-7.57 (m, 6H), 7.55-7.34 (m, 3H), 7.21-7.13 (m, 2H), 7.12-7.03 (m, 2H), 7.02-6.94 (m, 2H), 4.76-4.36 (m, 7H), 4.23-4.17 (m, 1H), 4.09-3.89 (m, 4H), 3.52-3.28 (m, 2H), 3.23-3.15 (m, 1H), 3.00-2.91 (m, 1H), 2.29-2.18 (m, 2H), 2.11-2.00 (m, 4H), 1.98-1.88 (m, 2H), 1.85-1.69 (m, 2H), 1.57-1.44 (m, 2H), 0.90-0.74 (m, 3H) |
| 433 | propyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(2- oxopyrrolidin-1- yl)phosphoryl)-L- alaninate | 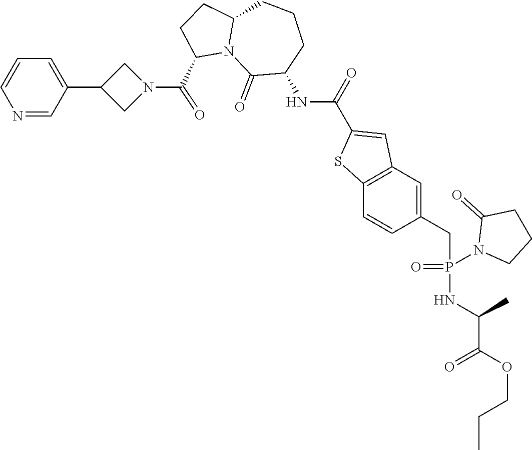 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)N1CCCC1=O] | 763.8 | ¹H NMR (400 MHz, CDCl₃) δ 8.68-8.49 (m, 2H), 7.96-7.61 (m, 5H), 7.45-7.31 (m, 2H), 4.75-4.37 (m, 4H), 4.33-3.81 (m, 8H), 3.66-3.42 (m, 3H), 3.00-2.82 (m, 1H), 2.51-2.14 (m, 5H), 2.11-1.98 (m, 5H), 1.96-1.91 (m, 2H), 1.72-1.61 (m, 4H), 1.42-1.34 (m, 3H), 0.98-0.87 (m, 3H) |
| 434 | propyl 1-((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino) cyclopropane-1- carboxylate | 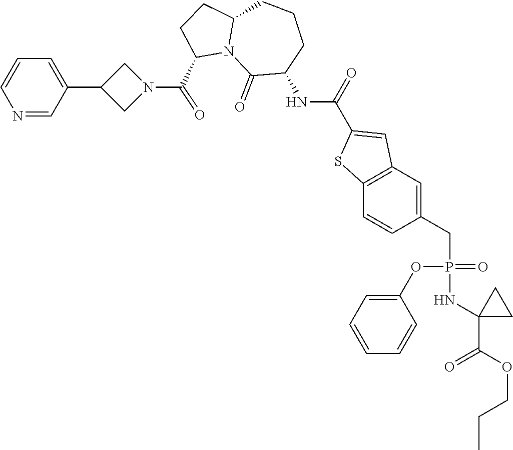 [SMILES: CCCOC(=O)C1(NP(=O)(Cc2ccc3sc(C(=O)N[C@H]4CCC[C@H]5CC[C@@H](C(=O)N6CC(c7cccnc7)C6)N5C4=O)cc3c2)Oc2ccccc2)CC1] | 784 | ¹H NMR (400 MHz, DMSO) δ 8.83-8.69 (m, 1H), 8.60-8.51 (m, 1H), 8.50-8.40 (m, 1H), 8.24-8.09 (m, 1H), 8.04-7.92 (m, 1H), 7.91-7.72 (m, 2H), 7.66-7.27 (m, 4H), 7.23-7.04 (m, 3H), 6.06 (d, J = 15.8 Hz, 1H), 4.86- 4.56 (m, 2H), 4.50- 4.17 (m, 3H), 4.09- 3.76 (m, 5H), 3.66- 3.40 (m, 2H), 2.27- 2.13 (m, 1H), 2.09- 1.93 (m, 2H), 1.92- 1.60 (m, 7H), 1.57- 1.41 (m, 2H), 1.27- 1.15 (m, 2H), 1.01- 0.74 (m, 5H) |
| 435 | propyl ((1-(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)ethyl)(phenoxy) phosphoryl)-L- alaninate | 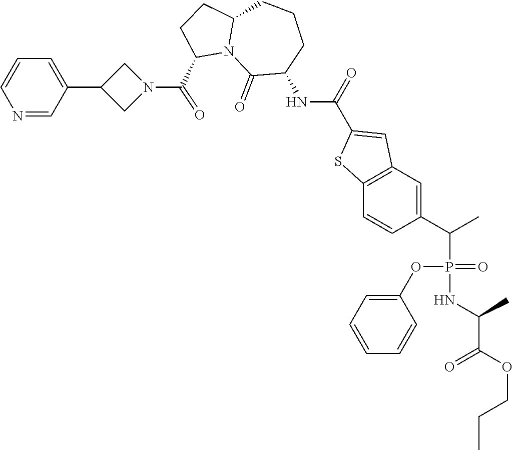 [SMILES: CCCOC(=O)[C@H](C)NP(=O)(Oc1ccccc1)C(C)c1ccc2sc(C(=O)N[C@H]3CCC[C@H]4CC[C@@H](C(=O)N5CC(c6cccnc6)C5)N4C3=O)cc2c1] | 786.4 | ¹H NMR (400 MHz, DMSO) δ 8.84-8.71 (m, 1H), 8.61-8.53 (m, 1H), 8.51-8.40 (m, 1H), 8.25-8.17 (m, 1H), 8.00-7.91 (m, 2H), 7.91-7.83 (m, 1H), 7.54-7.47 (m, 1H), 7.46-7.30 (m, 2H), 7.30-7.04 (m, 3H), 7.01 (t, J = 7.2 Hz, 1H), 5.71- 5.38 (m, 1H), 4.88- 4.56 (m, 2H), 4.51- 4.17 (m, 3H), 4.09- 3.66 (m, 6H), 3.61- 3.48 (m, 1H), 2.26- 2.13 (m, 1H), 2.09- 1.92 (m, 2H), 1.86- 1.37 (m, 12H), 1.17- 0.95 (m, 2H), 0.89- 0.66 (m, 4H) |
| 436 | neopentyl (((7- (((3S,6S,10aS)-5- oxo-3-(6-phenyl-4- azaspiro[2.4]heptane- 4- carbonyl)decahydro pyrrolo[1,2- a]azocin-6- yl)carbamoyl) naphthalen-2- yl)methyl)(phenoxy) phosphoryl)-L- alaninate | 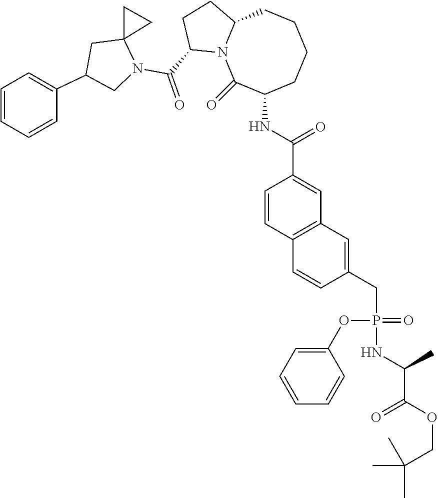 [SMILES: C[C@H](NP(=O)(Cc1ccc2ccc(C(=O)N[C@H]3CCCC[C@H]4CC[C@@H](C(=O)N5CC(c6ccccc6)CC56CC6)N4C3=O)cc2c1)Oc1ccccc1)C(=O)OCC(C)(C)C] | 847.5 | ¹H NMR (400 MHz, DMSO) δ 8.68-8.61 (m, 1H), 8.49-8.43 (m, 1H), 8.04-7.89 (m, 4H), 7.67-7.58 (m, 1H), 7.38-7.21 (m, 8H), 7.17-7.08 (m, 3H), 5.79-5.58 (m, 1H), 5.07-4.95 (m, 1H), 4.59-4.38 (m, 2H), 4.32-4.21 (m, 1H), 4.16-3.86 (m, 1H), 3.76-3.49 (m, 5H), 2.37-1.56 (m, 16H), 1.06 (2d, J = 7.1 Hz, 3H), 0.89- 0.72 (m, 9H), 0.53- 0.39 (m, 2H) |
| 437 | propyl ((2,2- difluoropropoxy)((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl)- L-alaninate | 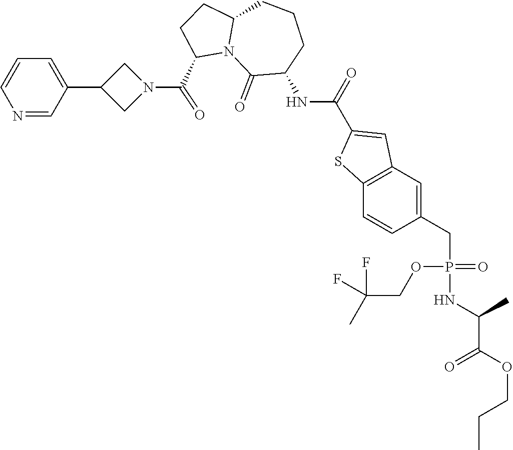 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)OCC(C)(F)F] | 774.8 | ¹H NMR (400 MHz, DMSO) δ 8.85-8.73 (m, 1H), 8.61-8.52 (m, 1H), 8.51-8.41 (m, 1H), 8.21-8.17 (m, 1H), 7.96-7.81 (m, 3H), 7.44-7.30 (m, 2H), 5.49-5.26 (m, 1H), 4.85-4.57 (m, 2H), 4.50-4.19 (m, 3H), 4.08-3.84 (m, 7H), 3.33-3.30 (m, 2H), 2.90-2.69 (m, 1H), 2.26-2.14 (m, 1H), 2.08-1.93 (m, 2H), 1.89-1.65 (m, 7H), 1.62-1.59 (m, 1H), 1.57-1.56 (m, 1H), 1.29-1.12 (m, 3H), 1.10-1.09 (m, 3H), 0.91-0.81 (m, 3H) |
| 438 | propyl ((2- methoxyethoxy)((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl)- L-alaninate | 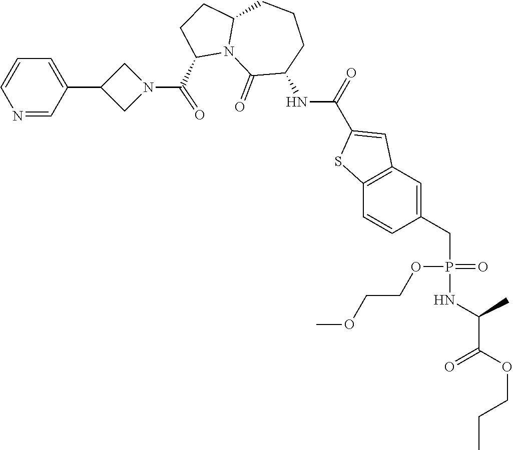 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)OCCOC] | 754.3 | ¹H NMR (400 MHz, DMSO) δ 8.84-8.72 (m, 1H), 8.59-8.53 (m, 1H), 8.50-8.42 (m, 1H), 8.21-8.14 (m, 1H), 7.96-7.79 (m, 3H), 7.46-7.29 (m, 2H), 5.21-5.07 (m, 1H), 4.83-4.60 (m, 2H), 4.48-4.21 (m, 3H), 4.04-3.83 (m, 7H), 3.53-3.41 (m, 2H), 3.33-3.32 (m, 3H), 3.26-3.22 (m, 3H), 2.26-2.12 (m, 1H), 2.07-1.91 (m, 2H), 1.90-1.65 (m, 7H), 1.60-1.48 (m, 2H), 1.27-1.03 (m, 3H), 0.90-0.80 (m, 3H) |
| 439 | propyl (ethoxy((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl)- L-alaninate | 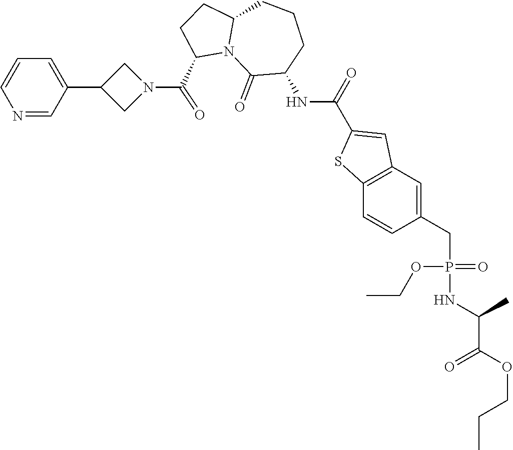 [SMILES: CCCOC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)OCC] | 724.4 | ¹H NMR (400 MHz, DMSO) δ 8.82-8.70 (m, 1H), 8.59-8.53 (m, 1H), 8.50-8.41 (m, 1H), 8.21-8.16 (m, 1H), 7.96-7.79 (m, 3H), 7.44-7.30 (m, 2H), 5.17-5.02 (m, 1H), 4.84-4.56 (m, 2H), 4.48-4.18 (m, 3H), 4.05-3.63 (m, 8H), 3.27-3.15 (m, 2H), 2.28-2.11 (m, 1H), 2.12-1.90 (m, 2H), 1.91-1.45 (m, 9H), 1.31-1.02 (m, 6H), 0.94-0.78 (m, 3H) |
| 440 | propyl ((fluoro(7- (((3S,6S,10aS)-3- (6-(1-methyl-2- oxo-1,2- dihydropyridin-4- yl)-4- azaspiro[2.4]heptane- 4-carbonyl)-5- oxodecahydropyrrolo [1,2-a]azocin-6- yl)carbamoyl) naphthalen-2- yl)methyl)(phenoxy) phosphoryl)-L- alaninate | 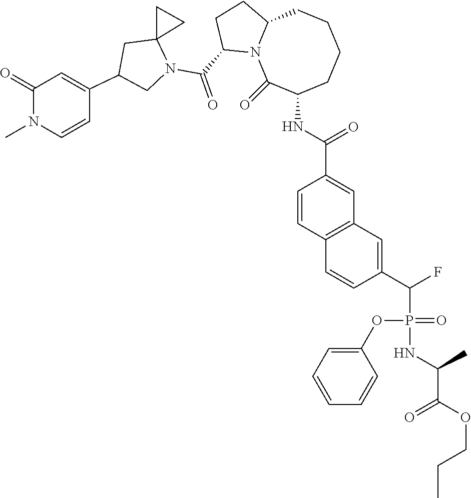 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5CC(c6ccn(C)c(=O)c6)CC56CC6)N4C3=O)cc2c1] | 868.5 | ¹H NMR (400 MHz, DMSO) δ 8.75-8.61 (m, 1H), 8.55 (s, 1H), 8.13 (s, 1H), 8.08- 7.97 (m, 3H), 7.81- 7.69 (m, 1H), 7.68- 7.50 (m, 1H), 7.42- 7.25 (m, 2H), 7.24- 7.08 (m, 3H), 6.49 6.05 (m, 4H), 5.13- 4.87 (m, 1H), 4.83- 3.52 (m, 8H), 3.40- 3.32 (m, 3H), 2.37- 2.12 (m, 1H), 2.11- 1.58 (m, 12H), 1.60- 1.33 (m, 6H), 1.22- 1.06 (m, 2H), 0.86- 0.68 (m, 3H), 0.55- 0.37 (m, 2H) |
| 441 | propyl ((fluoro(7- (((3S,6S,10aS)-3- ((R)-6-(1-methyl-2- oxo-1,2- dihydropyridin-4- yl)-4- azaspiro[2.4]heptane- 4-carbonyl)-5- oxodecahydropyrrolo [1,2-a]azocin-6- yl)carbamoyl) naphthalen-2- yl)methyl)(phenoxy) phosphoryl)-L- alaninate | 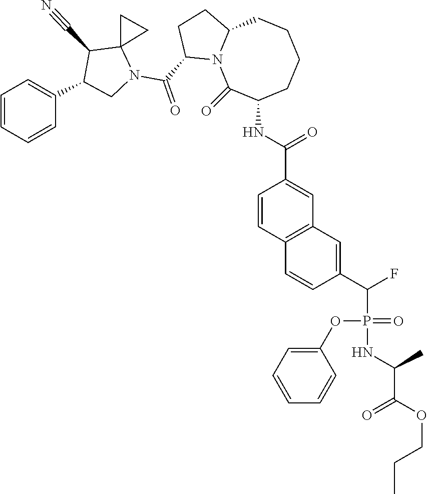 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5C[C@H](c6ccccc6)[C@@H](C#N)C56CC6)N4C3=O)cc2c1] | 868.4 | ¹H NMR (400 MHz, DMSO) δ 8.72-8.63 (m, 1H), 8.56 (s, 1H), 8.13 (s, 1H), 8.07- 7.98 (m, 3H), 7.78- 7.71 (m, 1H), 7.58 (d, J = 7.0 Hz, 1H), 7.39-7.30 (m, 2H), 7.22-7.11 (m, 3H), 6.39-6.11 (m, 4H), 5.08-4.96 (m, 1H), 4.49 (t, J = 8.0 Hz, 1H), 4.29 (t, J = 19.1, 9.0 Hz, 1H), 4.06 (t, J = 8.7 Hz, 1H), 3.95- 3.66 (m, 4H), 3.33 (d, J = 3.9 Hz, 4H), 2.35-2.21 (m, 1H), 2.13-1.95 (m, 5H), 1.93-1.70 (m, 6H), 1.69-1.60 (m, 1H), 1.60-1.46 (m, 4H), 1.45-1.36 (m, 1H), 1.19-0.94 (m, 3H), 0.86-0.73 (m, 3H), 0.55-0.38 (m, 2H) |
| 442 | propyl (((7- (((3S,6S,10aS)-3- ((6S,7R)-7-cyano- 6-phenyl-4- azaspiro[2.4]heptane- 4-carbonyl)-5- oxodecahydropyrrolo [1,2-a]azocin-6- yl)carbamoyl) naphthalen-2- yl)fluoromethyl) (phenoxy)phosphoryl)- L-alaninate | 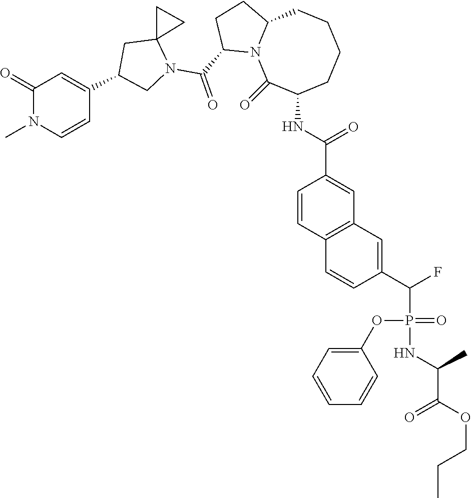 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5C[C@H](c6ccn(C)c(=O)c6)CC56CC6)N4C3=O)cc2c1] | 862.3 | ¹H NMR (400 MHz, DMSO) δ 8.79-8.65 (m, 1H), 8.57 (s, 1H), 8.13 (s, 1H), 8.08- 7.95 (m, 3H), 7.76 (d, J = 7.6 Hz, 1H), 7.54-7.44 (m, 2H), 7.42-7.26 (m, 5H), 7.25-7.08 (m, 3H), 6.39-6.10 (m, 2H), 5.15-4.92 (m, 1H), 4.61-4.16 (m, 3H), 4.09-3.64 (m, 6H), 2.33-1.34 (m, 16H), 1.20-1.08 (m, 2H), 0.96 (d, J = 7.1 Hz, 1H), 0.88-0.59 (m, 5H) |
| 443 | propyl ((fluoro(7- (((3S,6S,10aS)-3- ((S)-6-(1-methyl-2- oxo-1,2- dihydropyridin-4- yl)-4- azaspiro[2.4]heptane- 4-carbonyl)-5- oxodecahydropyrrolo [1,2-a]azocin-6- yl)carbamoyl) naphthalen-2- yl)methyl)(phenoxy) phosphoryl)-L- alaninate | 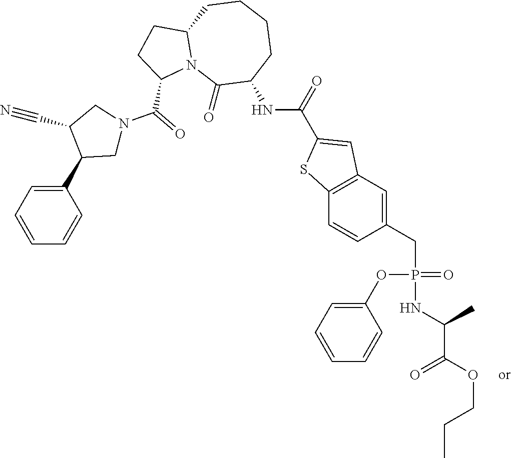 [SMILES: CCCOC(=O)[C@H](C)NP(=O)(Cc1ccc2sc(C(=O)N[C@H]3CCCC[C@H]4CC[C@@H](C(=O)N5C[C@@H](C#N)[C@H](c6ccccc6)C5)N4C3=O)cc2c1)Oc1ccccc1] 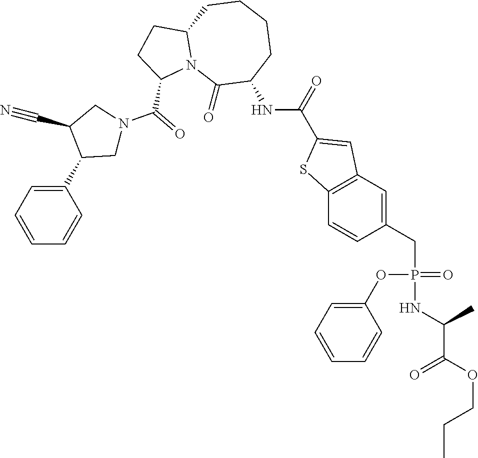 [SMILES: CCCOC(=O)[C@H](C)NP(=O)(Cc1ccc2sc(C(=O)N[C@H]3CCCC[C@H]4CC[C@@H](C(=O)N5C[C@H](c6ccccc6)[C@@H](C#N)C5)N4C3=O)cc2c1)Oc1ccccc1] | 868.6 | ¹H NMR (400 MHz, DMSO) δ 8.72-8.63 (m, 1H), 8.56 (s, 1H), 8.13 (s, 1H), 8.07- 7.98 (m, 3H), 7.78- 7.72 (m, 1H), 7.61 (d, J = 7.0 Hz, 1H), 7.38-7.30 (m, 2H), 7.22-7.11 (m, 3H), 6.39-6.12 (m, 4H), 5.07-4.97 (m, 1H), 4.53 (t, J = 8.3 Hz, 1H), 4.37-4.22 (m, 2H), 3.98-3.68 (m, 3H), 3.58 (t, J = 9.4 Hz, 1H), 3.36 (d, J = 2.5 Hz, 4H), 2.26- 2.11 (m, 2H), 2.09- 1.99 (m, 2H), 1.97- 1.60 (m, 9H), 1.59- 1.37 (m, 5H), 1.19- 0.94 (m, 3H), 0.84- 0.73 (m, 3H), 0.51- 0.42 (m, 2H) |
| 444 | propyl (((2- (((3S,6S,10aS)-3- ((3S,4R)-3-cyano- 4- phenylpyrrolidine- 1-carbonyl)-5- oxodecahydropyrrolo [1,2-a]azocin-6- yl)carbamoyl)benzo [b]thiophen-5- yl)methyl)(phenoxy) phosphoryl)-L- alaninate Or propyl (((2- (((3S,6S,10aS)-3- ((3R,4S)-3-cyano- 4- phenylpyrrolidine- 1-carbonyl)-5- oxodecahydropyrrolo [1,2-a]azocin-6- yl)carbamoyl)benzo [b]thiophen-5- yl)methyl)(phenoxy) phosphoryl)-L- alaninate | 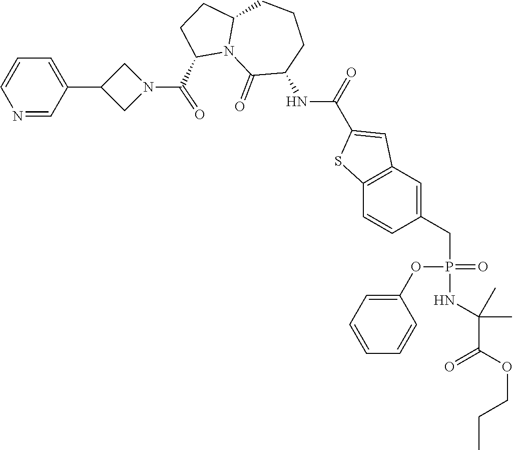 [SMILES: CCCOC(=O)C(C)(C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] Pyrrolidine building block SFC peak 2 was used for synthesis and biological testing | 824.5 | ¹H NMR (400 MHz, DMSO) δ 8.85-8.72 (m, 1H), 8.22 (d, J = 10.5 Hz, 1H), 7.94 (dd, J = 13.9, 6.0 Hz, 1H), 7.90-7.85 (m, 1H), 7.50-7.25 (m, 8H), 7.19-7.07 (m, 3H), 5.73-5.55 (m, 1H), 5.01-4.89 (m, 1H), 4.59-4.49 (m, 1H), 4.34-4.24 (m, 1H), 4.23-4.06 (m, 1H), 4.06-3.94 (m, 1H), 3.93-3.75 (m, 4H), 3.70-3.46 (m, 3H), 3.46-3.39 (m, 1H), 3.32-3.24 (m, 1H), 2.36-2.21 (m, 1H), 2.13-1.38 (m, 13H), 1.16-0.96 (m, 3H), 0.86-0.76 (m, 3H) |
| 445 | propyl 2-methyl-2- ((((2-(((3S,6S,9aS)- 5-oxo-3-(3- (pyridin-3- yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino) propanoate | 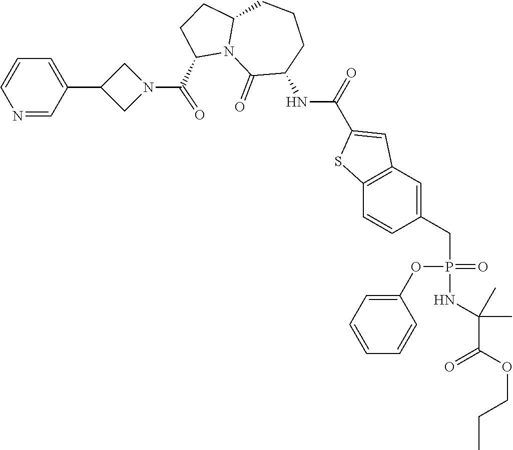 [SMILES: CCCOC(=O)C(C)(C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] | 786.4 | ¹H NMR (400 MHz, DMSO) δ 8.83-8.67 (m, 1H), 8.59-8.52 (m, 1H), 8.51-8.39 (m, 1H), 8.24-8.11 (m, 1H), 7.98-7.92 (m, 1H), 7.90-7.80 (m, 2H), 7.49-7.26 (m, 4H), 7.23-7.06 (m, 3H), 5.48-5.35 (m, 1H), 4.86-4.55 (m, 2H), 4.48-4.17 (m, 3H), 4.09-3.96 (m, 1H), 3.93-3.85 (m, 3H), 3.60-3.35 (m, 2H), 2.24-2.16 (m, 1H), 2.07-1.92 (m, 2H), 1.90-1.60 (m, 7H), 1.54-1.41 (m, 2H), 1.25 (s, 6H), 0.89-0.77 (m, 3H) |
| 446 | propyl ((S)-((R)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate Or propyl ((R)-((R)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 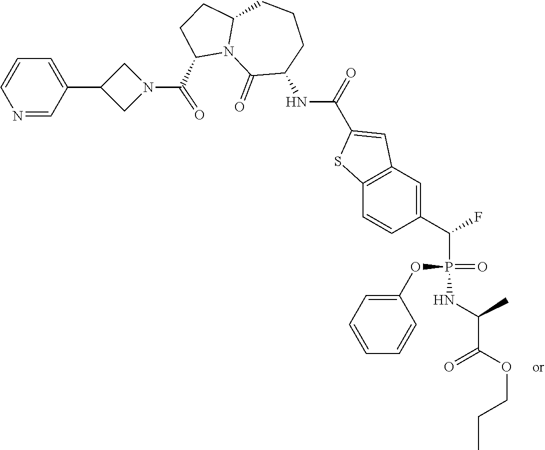 [SMILES: CCCOC(=O)[C@H](C)N[P@](=O)(Oc1ccccc1)[C@@H](F)c1ccc2sc(C(=O)N[C@H]3CCC[C@H]4CC[C@@H](C(=O)N5CC(c6cccnc6)C5)N4C3=O)cc2c1] | 790.3 | ¹H NMR (400 MHz, CDCl₃) δ 8.72-8.47 (m, 2H), 8.06-7.84 (m, 3H), 7.83-7.63 (m, 2H), 7.63-7.39 (m, 2H), 7.38-7.27 (m, 2H), 7.25 (d, J = 8.2 Hz, 1H), 7.22- 7.02 (m, 2H), 6.08- 5.86 (m, 1H), 5.08- 4.49 (m, 4H), 4.48- 3.83 (m, 7H), 3.72- 3.50 (m, 1H), 2.34- 2.15 (m, 3H), 2.08- 2.02 (m, 3H), 1.98- 1.67 (m, 4H), 1.67- 1.54 (m, 2H), 1.28 (d, J = 9.3 Hz, 2H), 1.12 (d, J = 7.1 Hz, 1H), 0.99-0.79 (m, 3H) |
| | | 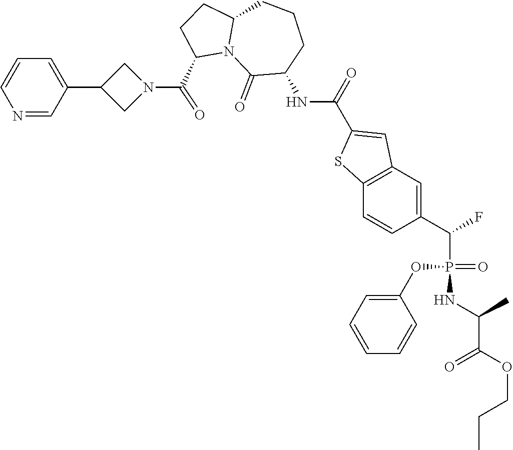 [SMILES: CCCOC(=O)[C@H](C)N[P@@](=O)(Oc1ccccc1)[C@@H](F)c1ccc2sc(C(=O)N[C@H]3CCC[C@H]4CC[C@@H](C(=O)N5CC(c6cccnc6)C5)N4C3=O)cc2c1] Linker phosphonamidate building block SFC peak 1 was used for synthesis and biological testing | | |
| 447 | propyl ((S)-((R)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate Or propyl ((R)-((R)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 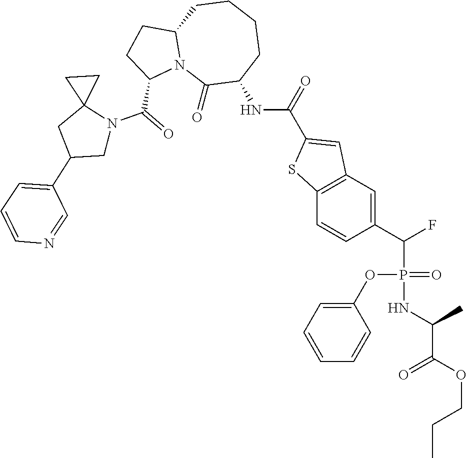 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CC(c6cccnc6)CC56CC6)N4C3=O)cc2c1] Linker phosphonamidate building block SFC peak 2 was used for synthesis and biological testing | 790.4 | ¹H NMR (400 MHz, CDCl₃) δ 9.08-8.80 (m, 1H), 8.76-8.63 (m, 1H), 8.51-8.10 (m, 1H), 8.02-7.61 (m, 5H), 7.54 (d, J = 8.4 Hz, 1H), 7.40- 7.30 (m, 2H), 7.29- 7.27 (m, 1H), 7.26- 7.12 (m, 2H), 5.95 (dd, J = 44.6, 6.7 Hz, 1H), 4.79-4.66 (m, 2H), 4.51-4.45 (m, 2H), 4.24-3.92 (m, 7H), 3.83-3.64 (m, 1H), 2.37-2.12 (m, 2H), 2.09-1.99 (m, 3H), 1.98-1.66 (m, 4H), 1.65-1.28 (m, 3H), 1.14 (t, J = 7.5 Hz, 3H), 0.90 (t, J = 7.3 Hz, 3H) |
| 448 | propyl ((fluoro(2- (((3S,6S,10aS)-5- oxo-3-(6-(pyridin- 3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 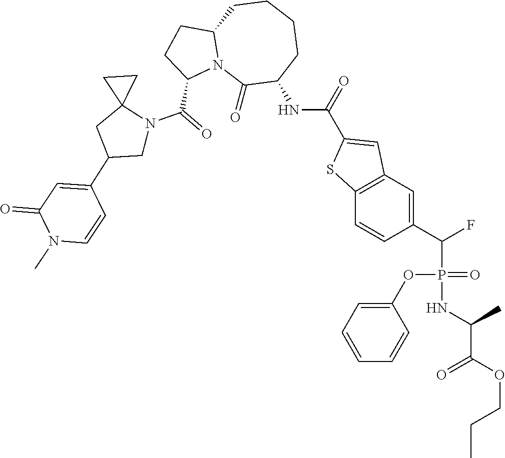 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CC(c6ccn(C)c(=O)c6)CC56CC6)N4C3=O)cc2c1] | 844.4 | ¹H NMR (400 MHz, DMSO) δ 8.96-8.80 (m, 1H), 8.79-8.57 (m, 2H), 8.38-8.26 (m, 1H), 8.22-8.02 (m, 3H), 7.75-7.56 (m, 2H), 7.43-7.30 (m, 2H), 7.26-7.10 (m, 3H), 6.33-6.06 (m, 2H), 5.04-4.88 (m, 1H), 4.58-4.43 (m, 2H), 4.32-4.12 (m, 2H), 4.09-3.90 (m, 4H), 2.39-1.43 (m, 17H), 1.28-1.11 (m, 3H), 1.00-0.92 (m, 1H), 0.88-0.74 (m, 3H), 0.56-0.38 (m, 2H). (TFA salt) |
| 449 | propyl ((fluoro(2- (((3S,6S,10aS)-3- (6-(1-methyl-2- oxo-1,2- dihydropyridin-4- yl)-4- azaspiro[2.4]heptane- 4-carbonyl)-5- oxodecahydropyrrolo [1,2-a]azocin-6- yl)carbamoyl)benzo [b]thiophen-5- yl)methyl)(phenoxy) phosphoryl)-L- alaninate | 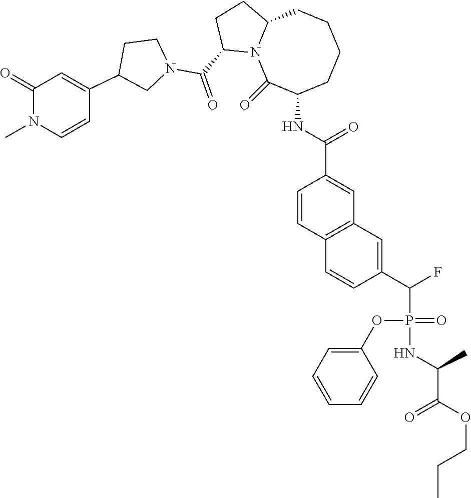 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5CCC(c6ccn(C)c(=O)c6)C5)N4C3=O)cc2c1] | 874.3 | ¹H NMR (400 MHz, DMSO) δ 8.93-8.77 (m, 1H), 8.33-8.25 (m, 1H), 8.15-8.00 (m, 2H), 7.67-7.55 (m, 2H), 7.41-7.27 (m, 2H), 7.25-7.08 (m, 3H), 6.35-6.06 (m, 4H), 5.01-4.87 (m, 1H), 4.59-3.99 (m, 3H), 3.97-3.51 (m, 4H), 3.40-3.34 (m, 4H), 2.31-1.71 (m, 13H), 1.58-1.40 (m, 5H), 1.18-1.10 (m, 2H), 0.99-0.75 (m, 4H), 0.53-0.41 (m, 2H) |
| 450 | propyl ((fluoro(7- (((3S,6S,10aS)-3- (3-(1-methyl-2- oxo-1,2- dihydropyridin-4- yl)pyrrolidine-1- carbonyl)-5- oxodecahydropyrrolo [1,2-a]azocin-6- yl)carbamoyl) naphthalen-2- yl)methyl)(phenoxy) phosphoryl)-L- alaninate | 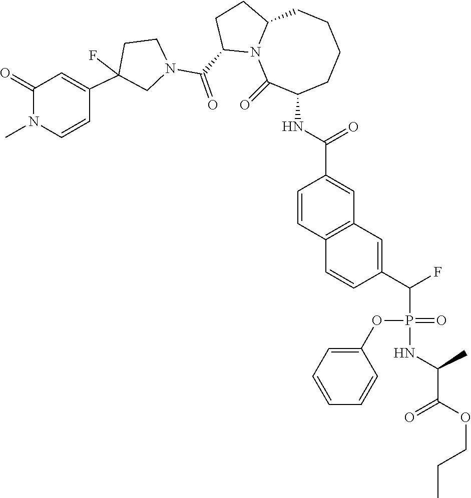 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5CCC(F)(c6ccn(C)c(=O)c6)C5)N4C3=O)cc2c1] | 842.3 | ¹H NMR (400 MHz, DMSO) δ 8.67-8.53 (m, 2H), 8.16-8.04 (m, 2H), 7.81-7.71 (m, 1H), 7.66-7.52 (m, 3H), 7.39-7.31 (m, 2H), 7.22-7.11 (m, 3H), 6.35-6.11 (m, 4H), 5.08-4.93 (m, 1H), 4.63-4.48 (m, 1H), 4.39-4.26 (m, 1H), 4.19-3.39 (m, 7H), 3.21-3.04 (m, 1H), 2.35-2.16 (m, 2H), 2.14-1.71 (m, 10H), 1.69-1.52 (m, 3H), 1.52-1.40 (m, 2H), 1.19-1.11 (m, 2H), 1.02-0.89 (m, 1H), 0.85-0.72 (m, 3H) |
| 451 | propyl ((fluoro(7- (((3S,6S,10aS)-3- (3-fluoro-3-(1- methyl-2-oxo-1,2- dihydropyridin-4- yl)pyrrolidine-1- carbonyl)-5- oxodecahydropyrrolo [1,2-a]azocin-6- yl)carbamoyl) naphthalen-2- yl)methyl)(phenoxy) phosphoryl)-L- alaninate | 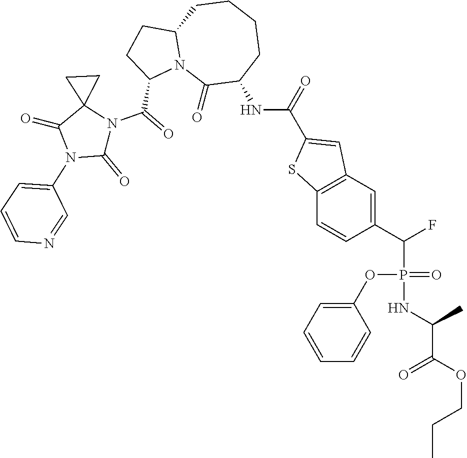 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6cccnc6)C(=O)C56CC6)N4C3=O)cc2c1] | 860.3 | ¹H NMR (400 MHz, DMSO) δ 8.75-8.48 (m, 2H), 8.16-7.95 (m, 4H), 7.80-7.59 (m, 2H), 7.35 (s, 2H), 7.26-7.07 (m, 3H), 6.69-6.11 (m, 4H), 5.09-4.92 (m, 1H), 4.81-3.94 (m, 4H), 3.93-3.50 (m, 5H), 3.45-3.36 (m, 3H), 2.41-2.20 (m, 2H), 2.12-1.77 (m, 8H), 1.71-1.36 (m, 6H), 1.20-0.93 (m, 3H), 0.88-0.71 (m, 3H) |
| 452 | propyl (((2- (((3S,6S,10aS)-3- (5,7-dioxo-6- (pyridin-3-yl)-4,6- diazaspiro[2.4] heptane-4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 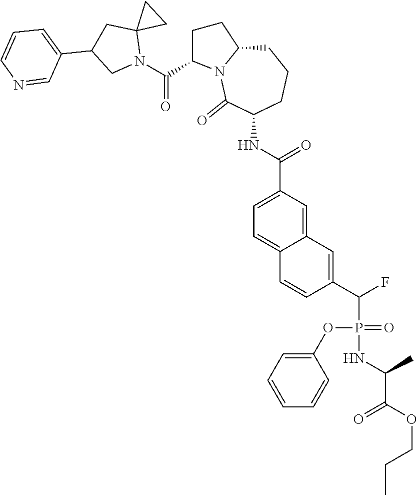 [SMILES: CCCOC(=O)[C@H](C)NP(=O)(Oc1ccccc1)C(F)c1ccc2ccc(C(=O)N[C@H]3CCC[C@H]4CC[C@@H](C(=O)N5CC(c6cccnc6)CC56CC6)N4C3=O)cc2c1] | 873.2 | ¹H NMR (400 MHz, DMSO) δ 9.05-8.59 (m, 3H), 8.32-7.91 (m, 4H), 7.69-7.51 (m, 2H), 7.41-7.09 (m, 5H), 6.32-6.05 (m, 2H), 5.44-5.31 (m, 1H), 5.00-4.87 (m, 1H), 4.41-4.29 (m, 1H), 4.01-3.62 (m, 3H), 2.47-2.06 (m, 4H), 1.95-1.37 (m, 14H), 1.21-0.91 (m, 3H), 0.89-0.75 (m, 3H) |
| 453 | propyl ((fluoro(7- (((3S,6S,9aS)-5- oxo-3-(6-(pyridin- 3-yl)-4- azaspiro[2.4]heptane- 4- carbonyl)octahydro- 1H-pyrrolo[1,2- a]azepin-6- yl)carbamoyl) naphthalen-2- yl)methyl)(phenoxy) phosphoryl)-L- alaninate | 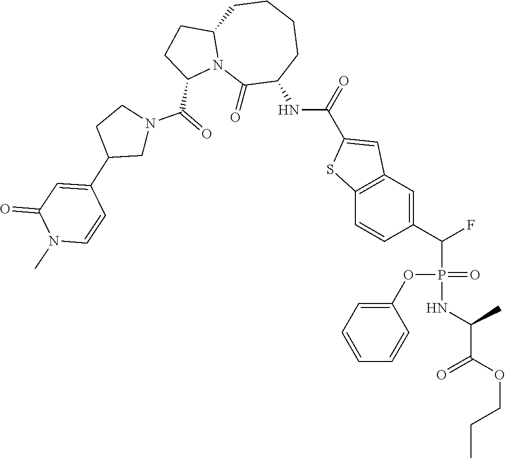 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CCC(c6ccn(C)c(=O)c6)C5)N4C3=O)cc2c1] | 824.4 | ¹H NMR (400 MHz, CDCl₃) δ 8.70-8.47 (m, 2H), 8.45-8.33 (m, 1H), 8.09-7.60 (m, 7H), 7.40-7.27 (m, 3H), 7.26-7.14 (m, 2H), 7.11-7.06 (m, 1H), 6.19-5.85 (m, 1H), 4.77-4.46 (m, 2H), 4.12-3.53 (m, 7H), 2.39-1.92 (m, 14H), 1.84-1.74 (m, 2H), 1.64-1.41 (m, 2H), 1.35-1.21 (m, 2H), 1.11-1.03 (m, 1H), 0.94-0.76 (m, 3H), 0.58-0.53 (m, 1H) |
| 454 | propyl ((fluoro(2- (((3S,6S,10aS)-3- (3-(1-methyl-2- oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 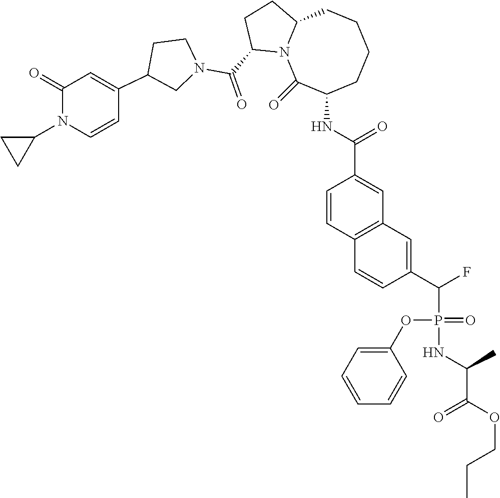 [SMILES: CCCOC(=O)[C@H](C)NP(=O)(Oc1ccccc1)C(F)c1ccc2ccc(C(=O)N[C@H]3CCCC[C@H]4CC[C@@H](C(=O)N5CCC(c6ccn(C7CC7)c(=O)c6)C5)N4C3=O)cc2c1] | 842.3 | ¹H NMR (400 MHz, DMSO) δ 8.67-8.52 (m, 2H), 8.16-8.12 (m, 1H), 8.08-7.97 (m, 3H), 7.78-7.72 (m, 1H), 7.64-7.60 (m, 1H), 7.39-7.31 (m, 2H), 7.22-7.12 (m, 3H), 6.38-6.12 (m, 4H), 5.08-4.93 (m, 1H), 4.63-4.48 (m, 1H), 4.38-4.10 (m, 1H), 3.97-3.47 (m, 6H), 3.44-3.35 (m, 3H), 3.21-3.04 (m, 1H), 2.35-2.16 (m, 2H), 2.14-1.71 (m, 10H), 1.69-1.52 (m, 3H), 1.52-1.40 (m, 2H), 1.19-1.11 (m, 2H), 1.02-0.89 (m, 1H), 0.85-0.72 (m, 3H) |
| 455 | propyl (((7- (((3S,6S,10aS)-3- (3-(1-cyclopropyl- 2-oxo-1,2- dihydropyridin-4- yl)pyrrolidine-1- carbonyl)-5- oxodecahydropyrrolo [1,2-a]azocin-6- yl)carbamoyl) naphthalen-2- yl)fluoromethyl) (phenoxy)phosphoryl)- L-alaninate | 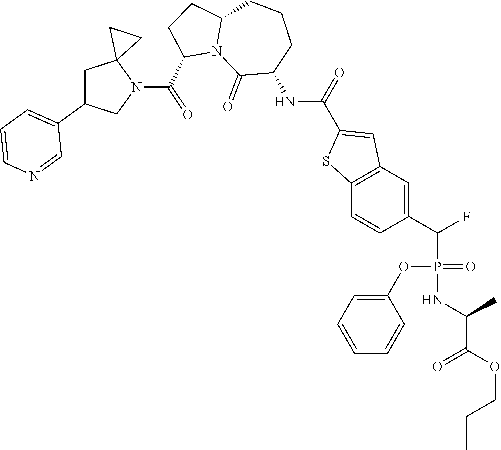 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cccnc6)CC56CC6)N4C3=O)cc2c1] | 868.3 | ¹H NMR (400 MHz, DMSO) δ 8.69-8.60 (m, 1H), 8.54 (s, 1H), 8.13 (s, 1H), 8.07- 7.99 (m, 3H), 7.75 (d, J = 7.9 Hz, 1H), 7.51-7.40 (m, 1H), 7.38-7.30 (m, 2H), 7.23-7.10 (m, 3H), 6.37-6.10 (m, 4H), 5.07-4.93 (m, 1H), 4.81-4.65 (m, 1H), 4.63-4.47 (m, 1H), 4.45-4.25 (m, 2H), 4.23-4.10 (m, 1H), 3.95-3.89 (m, 1H), 3.86-3.79 (m, 1H), 3.74-3.71 (m, 1H), 3.30-3.25 (m, 1H), 3.20-3.12 (m, 1H), 2.34-2.13 (m, 2H), 2.11-1.99 (m, 3H), 1.88-1.79 (m, 5H), 1.77-1.74 (m, 1H), 1.70-1.66 (m, 1H), 1.57-1.48 (m, 4H), 1.44-1.38 (m, 1H), 1.24-1.08 (m, 2H), 1.01-0.89 (m, 3H), 0.86-0.71 (m, 5H) |
| 456 | propyl ((fluoro(2- (((3S,6S,9aS)-5- oxo-3-(6-(pyridin- 3-yl)-4- azaspiro[2.4]heptane- 4- carbonyl)octahydro- 1H-pyrrolo[1,2- a]azepin-6- yl)carbamoyl)benzo [b]thiophen-5- yl)methyl)(phenoxy) phosphoryl)-L- alaninate | 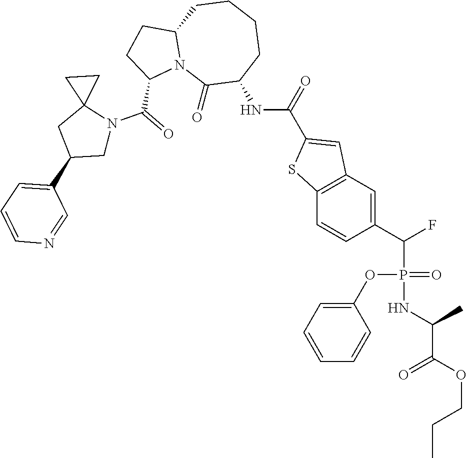 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C[C@@H](c6cccnc6)CC56CC6)N4C3=O)cc2c1] | 830.4 | ¹H NMR (400 MHz, CDCl₃) δ 8.77-8.46 (m, 2H), 8.03-7.51 (m, 6H), 7.51-7.27 (m, 3H), 7.23-7.16 (m, 1H), 7.12-7.05 (m, 1H), 6.07-5.84 (m, 1H), 4.76-4.45 (m, 2H), 4.16-3.51 (m, 7H), 2.40-2.31 (m, 1H), 2.29-2.13 (m, 4H), 2.09-1.94 (m, 7H), 1.84-1.74 (m, 2H), 1.65-1.49 (m, 2H), 1.32-1.24 (m, 2H), 1.17-1.06 (m, 1H), 0.95-0.81 (m, 3H), 0.62-0.46 (m, 2H) |
| 457 | propyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 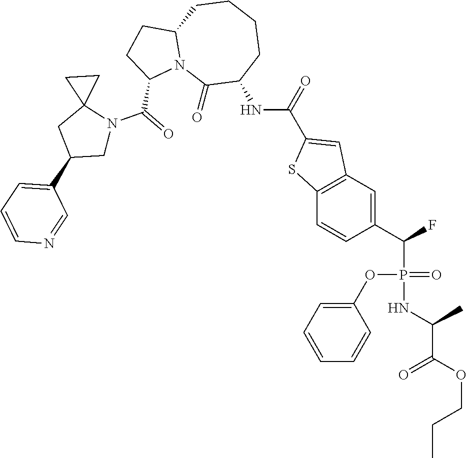 [SMILES: CCCOC(=O)[C@H](C)NP(=O)(Oc1ccccc1)[C@H](F)c1ccc2sc(C(=O)N[C@H]3CCCC[C@H]4CC[C@@H](C(=O)N5C[C@@H](c6cccnc6)CC56CC6)N4C3=O)cc2c1] | 844.3 | ¹H NMR (400 MHz, DMSO) δ 8.92-8.82 (m, 1H), 8.66-8.59 (m, 1H), 8.52-8.42 (m, 1H), 8.29 (s, 1H), 8.13-8.01 (m, 2H), 7.92-7.83 (m, 1H), 7.64-7.56 (m, 1H), 7.44-7.31 (m, 3H), 7.22-7.10 (m, 3H), 6.29-6.06 (m, 2H), 5.01-4.88 (m, 1H), 4.56-4.46 (m, 1H), 4.28-4.10 (m, 2H), 3.97-3.89 (m, 1H), 3.86-3.75 (m, 2H), 3.64-3.55 (m, 1H), 2.35-1.35 (m, 19H), 1.13 (d, J = 7.1 Hz, 3H), 0.88-0.73 (m, 3H), 0.54-0.36 (m, 2H) |
| 458 | propyl (((S)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 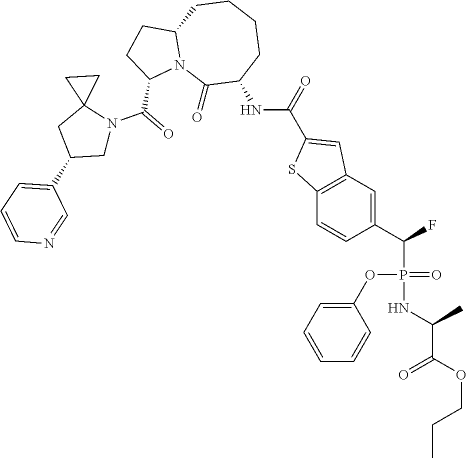 [SMILES: CCCOC(=O)[C@H](C)NP(=O)(Oc1ccccc1)[C@H](F)c1ccc2sc(C(=O)N[C@H]3CCCC[C@H]4CC[C@@H](C(=O)N5C[C@H](c6cccnc6)CC56CC6)N4C3=O)cc2c1] | 844.5 | ¹H NMR (400 MHz, DMSO) δ 8.92- 8.80 (m, 1H), 8.62 8.53 (m, 1H), 8.48 8.38 (m, 1H), 8.32 8.24 (m, 1H), 8.14 7.99 (m, 2H), 7.84- 7.75 (m, 1H), 7.65- 7.54 (m, 1H), 7.39- 7.30 (m, 3H), 7.24- 7.09 (m, 3H), 6.31- 6.05 (m, 2H), 5.05- 4.84 (m, 1H), 4.59- 4.40 (m, 1H), 4.29- 4.07 (m, 2H), 3.96- 3.79 (m, 3H), 3.71- 3.55 (m, 1H), 2.29- 1.45 (m, 19H), 1.17 (d, J = 7.1 Hz, 1H), 0.94 (d, J = 7.1 Hz, 2H), 0.83-0.75 (m, 3H), 0.62-0.28 (m, 2H) |
| 459 | propyl (((S)- fluoro(2- (((3S,6S,10aS)-5- oxo-3-((S)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 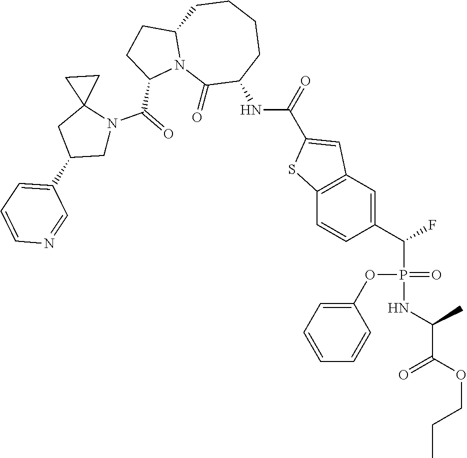 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccnc6)CC56CC6)N4C3=O)cc2c1] | 844.2 | ¹H NMR (400 MHz, DMSO) δ 8.87 (t, J = 7.0 Hz, 1H), 8.57 (d, J = 1.9 Hz, 1H), 8.45 (dd, J = 4.7, 1.5 Hz, 1H), 8.30 (s, 1H), 8.16-8.01 (m, 2H), 7.82-7.75 (m, 1H), 7.60 (t, J = 7.6 Hz, 1H), 7.41-7.30 (m, 3H), 7.23-7.08 (m, 3H), 6.32-6.19 (m, 1H), 6.19-6.07 (m, 1H), 5.00-4.90 (m, 1H), 4.54 (t, J = 8.4 Hz, 1H), 4.44 (t, J = 8.6 Hz, 1H), 4.27- 4.16 (m, 1H), 3.96- 3.48 (m, 5H), 2.37- 2.28 (m, 1H), 2.25- 2.15 (m, 1H), 2.12- 1.75 (m, 9H), 1.73- 1.44 (m, 7H), 1.17 (d, J = 7.1 Hz, 1H), 0.94 (d, J = 7.1 Hz, 2H), 0.86-0.75 (m, 3H), 0.55-0.42 (m, 2H) |
| 460 | propyl (((R)- fluoro(2- (((3S,6S,10aS)-5- oxo-3-((S)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 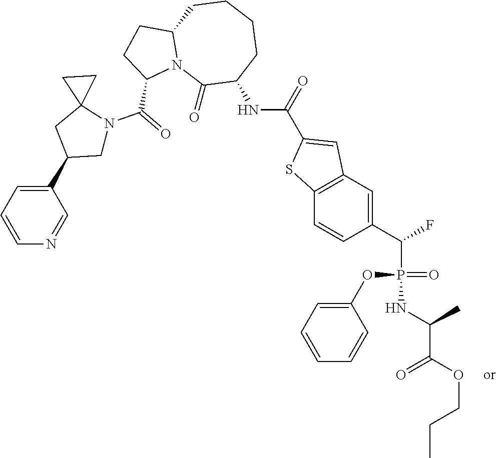 [SMILES: CCCOC(=O)[C@H](C)N[P@](=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | 844.3 | ¹H NMR (400 MHz, DMSO) δ 8.93-8.80 (m, 1H), 8.57 (d, J = 1.9 Hz, 1H), 8.45 (dd, J = 4.7, 1.5 Hz, 1H), 8.31 (s, 1H), 8.14-8.02 (m, 2H), 7.84-7.73 (m, 1H), 7.67-7.54 (m, 1H), 7.42-7.30 (m, 3H), 7.23-7.11 (m, 3H), 6.27-6.17 (m, 1H), 6.15-6.05 (m, 1H), 5.02-4.89 (m, 1H), 4.54 (t, J = 8.4 Hz, 1H), 4.45 (t, J = 8.6 Hz, 1H), 4.28-4.14 (m, 1H), 4.00-3.89 (m, 1H), 3.89-3.70 (m, 2H), 3.69-3.46 (m, 2H), 2.32 (t, J = 11.3 Hz, 1H), 2.25- 2.16 (m, 1H), 2.11- 1.75 (m, 9H), 1.73- 1.40 (m, 7H), 1.14 (d, J = 6.2 Hz, 3H), 0.88-0.73 (m, 3H), 0.56-0.42 (m, 2H) |
| | | 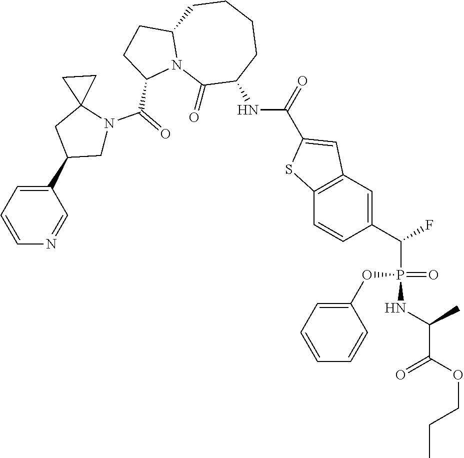 [SMILES: CCCOC(=O)[C@H](C)N[P@@](=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | | |
| 461 | propyl ((S)-((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate Or propyl ((R)-((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo | 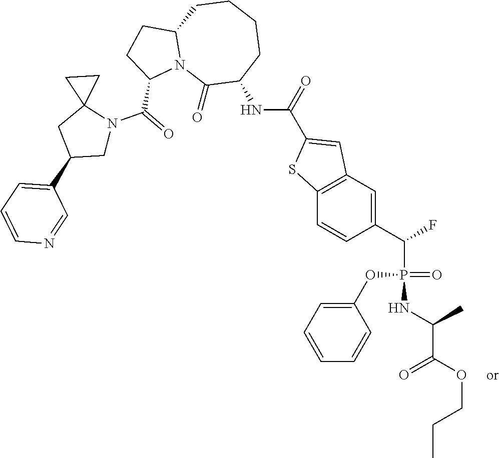 [SMILES: CCCOC(=O)[C@H](C)N[P@@](=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | | ¹H NMR (400 MHz, DMSO) δ 8.90-8.29 (m, 3H), 8.13-7.87 (m, 3H), 7.64-7.58 (m, 1H), 7.46-7.31 (m, 3H), 7.22-7.11 (m, 3H), 6.29-6.04 (m, 2H), 5.01-4.88 (m, 1H), 4.57-4.45 (m, 1H), 4.28-4.09 (m, 2H), 3.87-3.73 (m, 4H), 3.66-3.55 (m, 1H), 2.29-1.37 (m, 18H), 1.18-1.06 (m, 3H), 0.81-0.73 (m, 3H), 0.52-0.39 (m, 2H) |
| | [b]thiophen-5- yl)methyl)(phenoxy) phosphoryl)-L- alaninate | 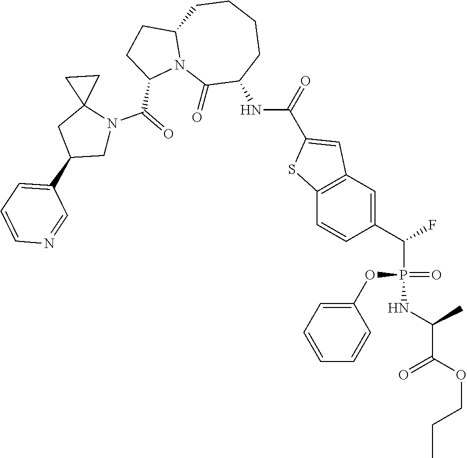 [SMILES: CCCOC(=O)[C@H](C)N[P@](=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] Linker phosphonamidate building block SFC peak 1 was used for synthesis and biological testing | | |
| 462 | propyl ((S)-((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate Or propyl ((R)-((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo | 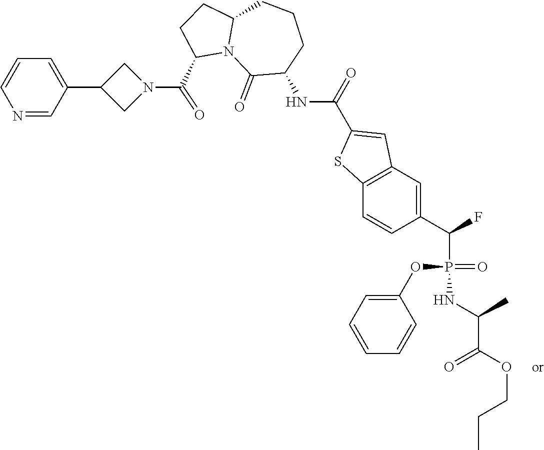 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cccnc6)C5)N4C3=O)cc2c1] | 844.3 | ¹H NMR (400 MHz, DMSO) δ 8.95-7.52 (m, 8H), 7.42-7.31 (m, 3H), 7.23-7.12 (m, 3H), 6.28-6.09 (m, 2H), 5.00-4.90 (m, 1H), 4.57-4.47 (m, 1H), 4.29-4.08 (m, 2H), 3.98-3.72 (m, 4H), 3.64-3.53 (m, 1H), 2.27-1.46 (m, 18H), 1.14 (d, J = 7.1 Hz, 3H), 0.83 (t, J = 7.4 Hz, 3H), 0.52- 0.34 (m, 2H) |
| | [b]thiophen-5- yl)methyl)(phenoxy) phosphoryl)-L- alaninate | 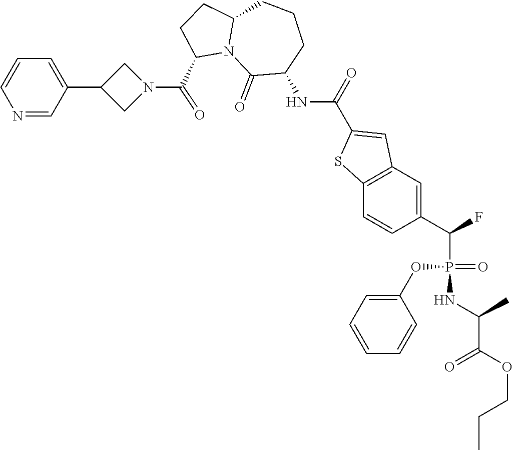 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cccnc6)C5)N4C3=O)cc2c1] Linker phosphonamidate building block SFC peak 2 was used for synthesis and biological testing | | |
| 463 | propyl ((S)-((S)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate Or propyl ((R)-((S)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 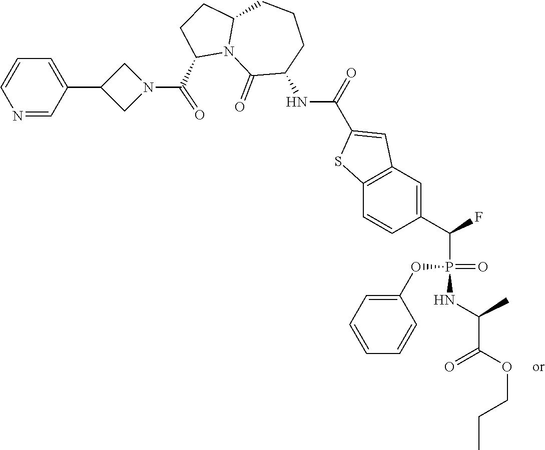 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cccnc6)C5)N4C3=O)cc2c1] | 790.2 | ¹H NMR (400 MHz, DMSO) δ 8.90-8.78 (m, 1H), 8.59-8.52 (m, 1H), 8.50-8.40 (m, 1H), 8.32-8.26 (m, 1H), 8.13-8.04 (m, 2H), 7.90-7.81 (m, 1H), 7.65-7.58 (m, 1H), 7.44-7.30 (m, 3H), 7.24-7.10 (m, 3H), 6.32-6.08 (m, 1H), 4.83-4.57 (m, 2H), 4.47-4.19 (m, 3H), 4.09-3.81 (m, 6H), 3.63-3.57 (m, 1H), 2.09-1.92 (m, 2H), 1.90-1.66 (m, 8H), 1.54-1.42 (m, 2H), 1.20- 0.93 (m, 3H), 0.85-0.77 (m, 3H) |
| | | 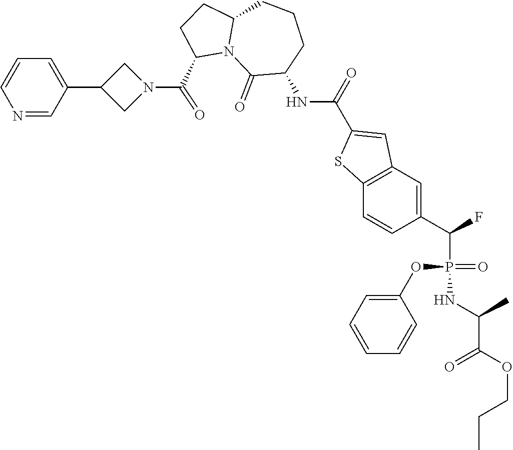 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cccnc6)C5)N4C3=O)cc2c1] Linker phosphonamidate building block SFC peak 1 was used for synthesis and biological testing | | |
| 464 | propyl ((S)-((S)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate Or propyl ((R)-((S)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 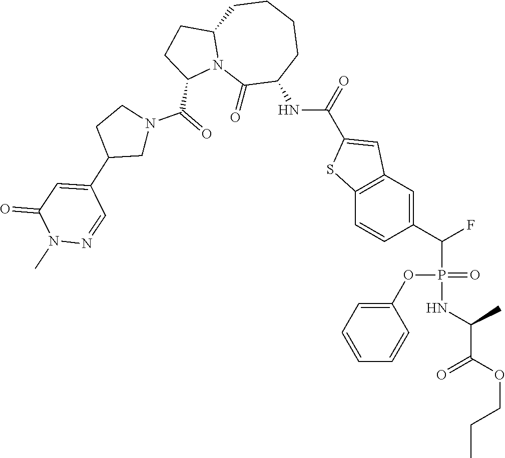 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CCC(c6cnn(C)c(=O)c6)C5)N4C3=O)cc2c1] Linker phosphonamidate building block SFC peak 2 was used for synthesis and biological testing | 790.2 | ¹H NMR (400 MHz, DMSO) δ 8.90-8.78 (m, 1H), 8.59-8.53 (m, 1H), 8.51-8.40 (m, 1H), 8.31-8.26 (m, 1H), 8.10-8.07 (m, 1H), 7.90-7.83 (m, 1H), 7.70-7.57 (m, 2H), 7.42-7.33 (m, 3H), 7.20-7.10 (m, 3H), 6.39-6.28 (m, 1H), 6.20-6.18 (m, 1H), 4.84-4.57 (m, 2H), 4.47-4.18 (m, 3H), 4.05-3.81 (m, 6H), 2.06-1.91 (m, 2H), 1.89-1.58 (m, 8H), 1.52-1.42 (m, 2H), 1.18 (d, J = 7.1 Hz, 3H), 0.85- 0.78 (m, 3H) |
| 465 | propyl ((fluoro(2- (((3S,6S,10aS)-3- (3-(1-methyl-6- oxo-1,6- dihydropyridazin-4- yl)pyrrolidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 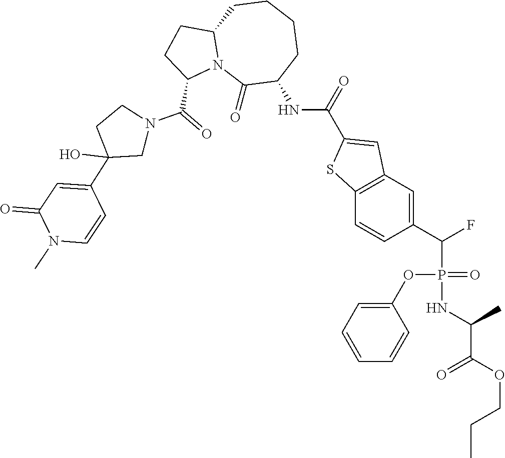 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CCC(O)(c6ccn(C)c(=O)c6)C5)N4C3=O)cc2c1] | 849.2 | ¹H NMR (400 MHz, DMSO) δ 8.88-8.77 (m, 1H), 8.33-8.27 (m, 1H), 8.14-8.03 (m, 2H), 7.96-7.87 (m, 1H), 7.65-7.57 (m, 1H), 7.40-7.31 (m, 2H), 7.23-7.09 (m, 3H), 6.83-6.75 (m, 1H), 6.28-6.08 (m, 1H), 5.02-4.88 (m, 1H), 4.61-4.49 (m, 1H), 4.33-4.14 (m, 1H), 4.00-3.39 (m, 9H), 3.25-3.06 (m, 1H), 2.53-2.52 (m, 1H), 2.29-2.21 (m, 1H), 2.12-1.39 (m, 15H), 1.19-0.92 (m, 3H), 0.87-0.74 (m, 3H) |
| 466 | propyl ((fluoro(2- (((3S,6S,10aS)-3- (3-hydroxy-3-(1- methyl-2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 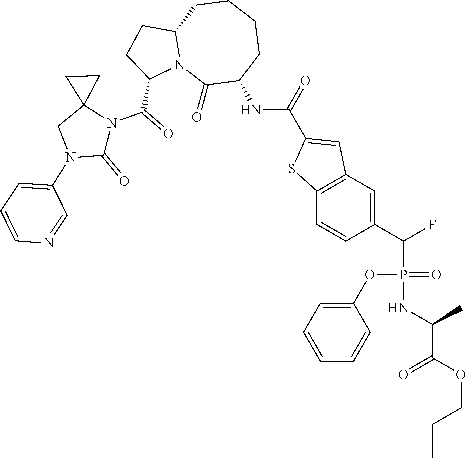 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6cccnc6)CC56CC6)N4C3=O)cc2c1] | 864.2 | ¹H NMR (400 MHz, DMSO) δ 8.81 (s, 1H), 8.30 (s, 1H), 8.14-8.03 (m, 2H), 7.67-7.56 (m, 2H), 7.40-7.30 (m, 2H), 7.24-7.08 (m, 3H), 6.54-6.43 (m, 1H), 6.38-6.05 (m, 3H), 5.66-5.44 (m, 1H), 5.02-4.89 (m, 1H), 4.66-4.44 (m, 1H), 4.35-4.19 (m, 1H), 4.05-3.53 (m, 6H), 3.51-3.35 (m, 4H), 2.30-1.69 (m, 11H), 1.68-1.38 (m, 5H), 1.21-0.91 (m, 3H), 0.87-0.75 (m, 3H) |
| 467 | propyl ((fluoro(2- (((3S,6S,10aS)-5- oxo-3-(5-oxo-6- (pyridin-3-yl)-4,6- diazaspiro[2.4] heptane-4- carbonyl)decahydro pyrrolo[1,2- a]azocin-6- yl)carbamoyl)benzo [b]thiophen-5- yl)methyl)(phenoxy) phosphoryl)-L- alaninate | 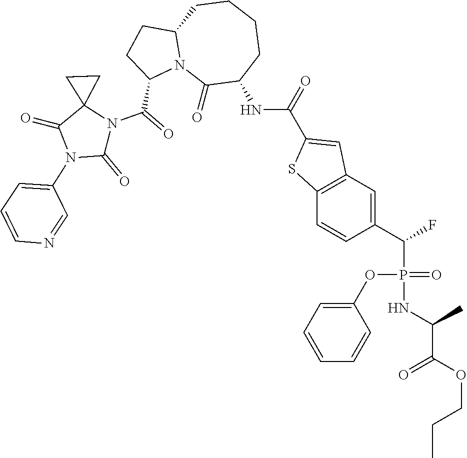 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C(=O)N(c6cccnc6)C(=O)C56CC6)N4C3=O)cc2c1] | 859.3 | ¹H NMR (400 MHz, DMSO) δ 8.95- 7.52 (m, 8H), 7.42- 7.31 (m, 3H), 7.23- 7.12 (m, 3H), 6.28- 6.09 (m, 2H), 5.00- 4.90 (m, 1H), 4.57- 4.47 (m, 1H), 4.29- 4.08 (m, 2H), 3.98- 3.72 (m, 4H), 3.64- 3.53 (m, 1H), 2.27- 1.46 (m, 18H), 1.14 (d, J = 7.1 Hz, 3H), 0.83 (t, J = 7.4 Hz, 3H), 0.52-0.34 (m, 2H) |
| 468 | propyl (((R)-(2- (((3S,6S,10aS)-3- (5,7-dioxo-6- (pyridin-3-yl)-4,6- diazaspiro[2.4] heptane-4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 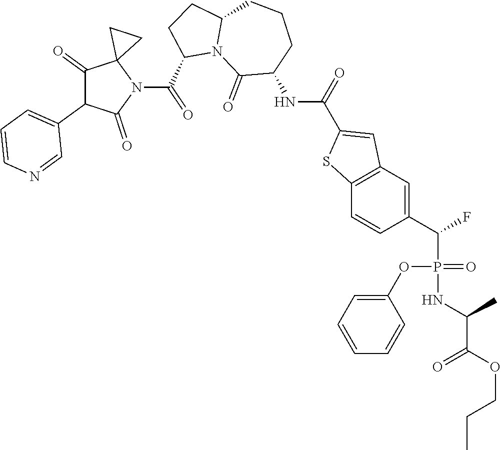 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C(=O)C(c6cccnc6)C(=O)C56CC6)N4C3=O)cc2c1] | 873.2 | ¹H NMR (400 MHz, DMSO) δ 8.95-8.62 (m, 3H), 8.32-8.22 (m, 1H), 8.13-8.01 (m, 1H), 7.99-7.90 (m, 1H), 7.68-7.53 (m, 3H), 7.42-7.30 (m, 2H), 7.21-7.09 (m, 3H), 6.27-6.04 (m, 2H), 5.41-5.32 (m, 1H), 5.01-4.82 (m, 1H), 4.40-4.31 (m, 1H), 3.97-3.70 (m, 3H), 2.21-1.37 (m, 18H), 1.18-1.10 (m, 3H), 0.87-0.72 (m, 3H) |
| 469 | propyl (((R)-(2- (((3S,6S,9aS)-3- (5,7-dioxo-6- (pyridin-3-yl)-4,6- diazaspiro[2.4] heptane-4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 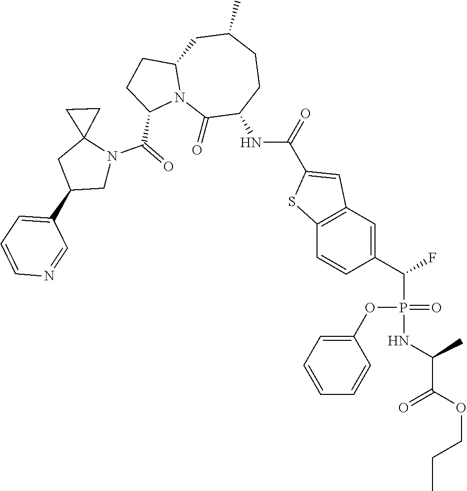 [SMILES: CCCOC(=O)[C@H](C)NP(=O)(Oc1ccccc1)[C@@H](F)c1ccc2sc(C(=O)N[C@H]3CC[C@@H](C)C[C@H]4CC[C@@H](C(=O)N5C[C@@H](c6cccnc6)CC56CC6)N4C3=O)cc2c1] | 859.3 | ¹H NMR (400 MHz, DMSO) δ 8.88-8.80 (m, 1H), 8.72-8.63 (m, 2H), 8.29-8.21 (m, 1H), 8.12-8.01 (m, 2H), 7.98-7.89 (m, 1H), 7.65-7.54 (m, 2H), 7.39-7.30 (m, 2H), 7.22-7.12 (m, 3H), 6.29-6.17 (m, 1H), 6.16-6.04 (m, 1H), 5.56-5.45 (m, 1H), 4.71-4.57 (m, 1H), 4.07-3.72 (m, 4H), 2.37-2.06 (m, 5H), 2.04-1.49 (m, 7H), 1.48-1.37 (m, 3H), 1.27-1.05 (m, 4H), 0.87-0.72 (m, 3H) |
| 470 | propyl (((R)- fluoro(2- (((3S,6S,9R,10aR)- 9-methyl-5-oxo-3- ((R)-6-(pyridin-3- yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 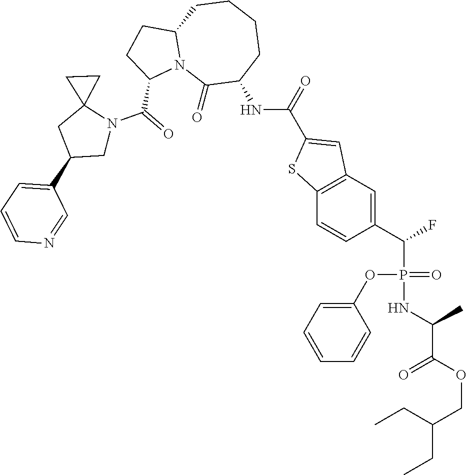 [SMILES: CCC(CC)COC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | 858.3 | ¹H NMR (400 MHz, DMSO) δ 8.80-8.67 (m, 1H), 8.61-8.54 (m, 1H), 8.45-8.38 (m, 1H), 8.30-8.18 (m, 1H), 8.09-7.84 (m, 2H), 7.81-7.75 (m, 1H), 7.63-7.28 (m, 5H), 7.21-7.10 (m, 3H), 6.27-6.10 (m, 1H), 4.83-4.72 (m, 1H), 4.48 (t, J = 8.7 Hz, 1H), 4.34- 4.26 (m, 1H), 4.14 (t, J = 8.4 Hz, 1H), 3.96- 3.74 (m, 4H), 3.63- 3.51 (m, 1H), 2.22- 2.06 (m, 2H), 2.05- 1.95 (m, 3H), 1.85- 1.39 (m, 10H), 1.27- 1.09 (m, 5H), 0.98- 0.92 (m, 3H), 0.85- 0.74 (m, 3H), 0.55- 0.40 (m, 2H) |
| 471 | 2-ethylbutyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 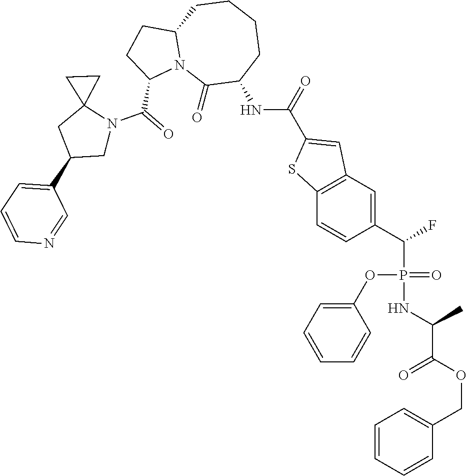 [SMILES: C[C@H](NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1)C(=O)OCc1ccccc1] | 886.3 | ¹H NMR (400 MHz, CDCl₃) δ 8.68-8.59 (m, 1H), 8.50-8.40 (m, 1H), 7.95-7.84 (m, 2H), 7.82-7.62 (m, 3H), 7.59-7.51 (m, 1H), 7.27-7.20 (m, 3H), 7.14-7.03 (m, 3H), 6.05-5.86 (m, 1H), 5.22-5.05 (m, 1H), 4.60-4.47 (m, 1H), 4.35-4.23 (m, 1H), 4.19-3.90 (m, 5H), 3.85-3.69 (m, 1H), 3.65-3.48 (m, 1H), 2.39-2.31 (m, 1H), 2.28-1.94 (m, 9H), 1.91-1.70 (m, 4H), 1.69-1.55 (m, 2H), 1.53-1.43 (m, 1H), 1.36-1.26 (m, 7H), 0.93-0.82 (m, 6H), 0.56-0.44 (m, 2H) |
| 472 | benzyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 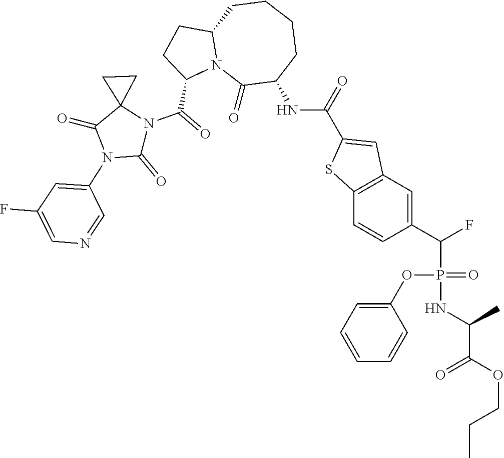 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6cncc(F)c6)C(=O)C56CC6)N4C3=O)cc2c1] | 892.3 | ¹H NMR (400 MHz, CDCl₃) δ 8.64-8.55 (m, 1H), 8.45-8.38 (m, 1H), 7.97-7.74 (m, 3H), 7.73-7.58 (m, 2H), 7.54-7.40 (m, 1H), 7.39-7.28 (m, 5H), 7.26-7.13 (m, 3H), 7.13-7.02 (m, 2H), 6.03-5.77 (m, 1H), 5.22-5.01 (m, 3H), 4.60-4.49 (m, 1H), 4.35-4.24 (m, 1H), 4.23-3.89 (m, 3H), 3.88-3.69 (m, 1H), 3.60-3.46 (m, 1H), 2.39-2.28 (m, 1H), 2.26-1.94 (m, 8H), 1.92-1.68 (m, 4H), 1.67-1.52 (m, 2H), 1.37-1.22 (m, 3H), 1.19-1.12 (m, 1H), 0.55-0.41 (m, 2H) |
| 473 | propyl ((fluoro(2- (((3S,6S,10aS)-3- (6-(5-fluoropyridin- 3-yl)-5,7-dioxo-4,6- diazaspiro[2.4] heptane-4-carbonyl)-5- oxodecahydropyrrolo [1,2-a]azocin-6- yl)carbamoyl)benzo [b]thiophen-5- yl)methyl)(phenoxy) phosphoryl)-L- alaninate | 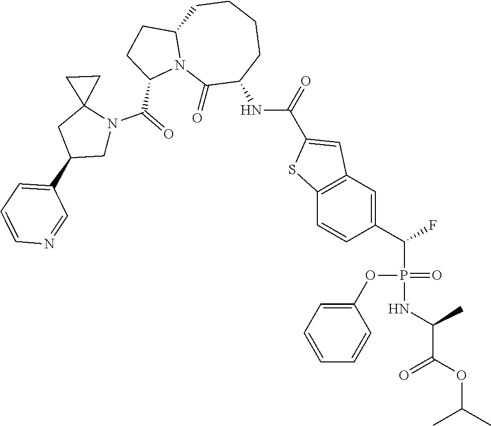 [SMILES: CC(C)OC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1] | 891.4 | ¹H NMR (400 MHz, DMSO) δ 8.94-7.93 (m, 7H), 7.72-7.54 (m, 1H), 7.42-7.10 (m, 5H), 6.30-6.00 (m, 2H), 5.45-5.28 (m, 1H), 4.99-4.84 (m, 1H), 4.51-4.26 (m, 1H), 3.99-3.65 (m, 3H), 2.47-2.04 (m, 4H), 1.95-1.38 (m, 14H), 1.18-1.08 (m, 3H), 0.87-0.73 (m, 3H) |
| 474 | isopropyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 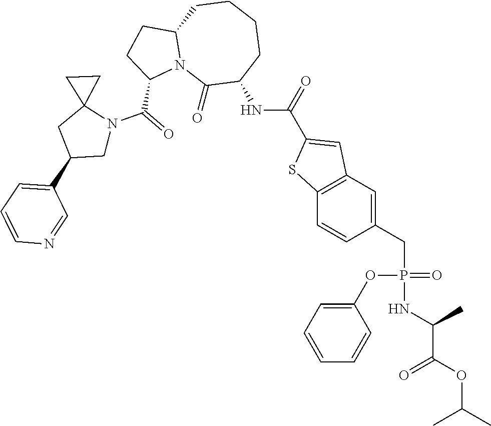 [SMILES: CC(C)OC(=O)[C@H](C)NP(=O)(Cc1ccc2sc(C(=O)N[C@H]3CCCC[C@H]4CC[C@@H](C(=O)N5C[C@@H](c6cccnc6)CC56CC6)N4C3=O)cc2c1)Oc1ccccc1] | 844.2 | ¹H NMR (400 MHz, DMSO) δ 8.91-8.81 (m, 1H), 8.58 (s, 1H), 8.46-8.39 (m, 1H), 8.29 (s, 1H), 8.11- 8.02 (m, 2H), 7.78 (d, J = 7.9 Hz, 1H), 7.61 (d, J = 8.4 Hz, 1H), 7.40-7.28 (m, 3H), 7.21-7.10 (m, 3H), 6.27-6.03 (m, 2H), 4.99-4.90 (m, 1H), 4.88-4.69 (m, 1H), 4.51 (t, J = 8.3 Hz, 1H), 4.29-4.09 (m, 2H), 3.85-3.68 (m, 2H), 3.62-3.52 (m, 1H), 2.34-1.64 (m, 14H), 1.57-1.45 (m, 2H), 1.15-1.00 (m, 9H), 0.53-0.39 (m, 2H) |
| 475 | isopropyl (((2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 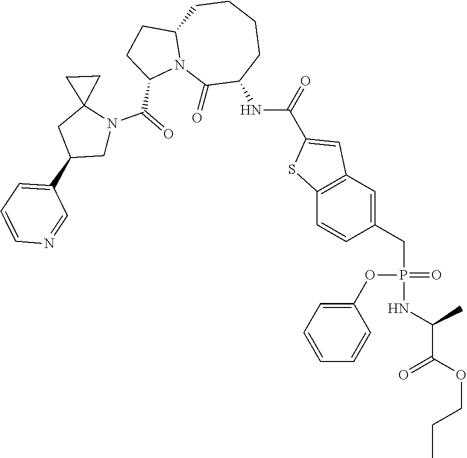 [SMILES: CCCOC(=O)[C@H](C)NP(=O)(Cc1ccc2sc(C(=O)N[C@H]3CCCC[C@H]4CC[C@@H](C(=O)N5C[C@@H](c6cccnc6)CC56CC6)N4C3=O)cc2c1)Oc1ccccc1] | 826.2 | ¹H NMR (400 MHz, DMSO) δ 8.84-8.79 (m, 1H), 8.58 (s, 1H), 8.44-8.40 (m, 1H), 8.21 (s, 1H), 7.95- 7.86 (m, 2H), 7.79- 7.75 (m, 1H), 7.66- 7.54 (m, 2H), 7.45- 7.40 (m, 1H), 7.35- 7.28 (m, 3H), 7.17- 7.09 (m, 3H), 5.69- 5.50 (m, 1H), 4.98- 4.88 (m, 1H), 4.84- 4.73 (m, 1H), 4.56- 4.45 (m, 1H), 4.28- 4.08 (m, 2H), 3.85- 3.68 (m, 2H), 3.62- 3.40 (m, 3H), 2.34- 1.48 (m, 16H), 1.12- 0.96 (m, 9H), 0.53- 0.37 (m, 2H) |
| 476 | propyl (((2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 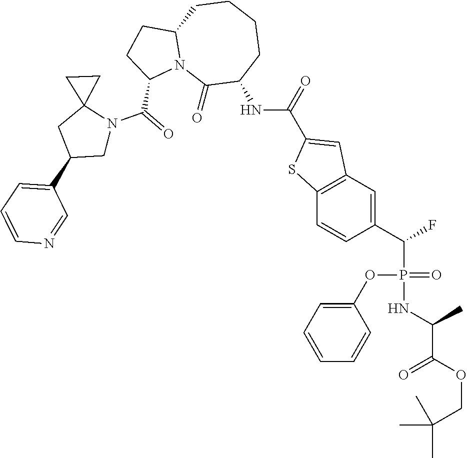 [SMILES: C[C@H](NP(=O)(Oc1ccccc1)[C@@H](F)c1ccc2sc(C(=O)N[C@H]3CCCC[C@H]4CC[C@@H](C(=O)N5C[C@@H](c6cccnc6)CC56CC6)N4C3=O)cc2c1)C(=O)OCC(C)(C)C] | 826.2 | ¹H NMR (400 MHz, DMSO) δ 8.85-8.63 (m, 3H), 8.37-8.15 (m, 2H), 7.97-7.70 (m, 3H), 7.43 (d, J = 8.3 Hz, 1H), 7.36- 7.28 (m, 2H), 7.17- 7.08 (m, 3H), 5.73- 5.57 (m, 1H), 4.98- 4.86 (m, 1H), 4.51 (t, J = 8.2 Hz, 1H), 4.28- 4.11 (m, 2H), 3.94- 3.64 (m, 5H), 3.52- 3.33 (m, 2H), 2.34- 2.14 (m, 3H), 2.05- 1.71 (m, 9H), 1.68- 1.41 (m, 6H), 1.10- 0.97 (m, 3H), 0.84- 0.77 (m, 3H), 0.55- 0.38 (m, 2H) |
| 477 | neopentyl (((R)- fluoro(2- (((3S,6S,10aS)-5- oxo-3-((R)-6- (pyridin-3-yl)-4- azaspiro[2.4]heptane- 4- carbonyl)decahydro pyrrolo[1,2- a]azocin-6- yl)carbamoyl)benzo [b]thiophen-5- yl)methyl)(phenoxy) phosphoryl)-L- alaninate | 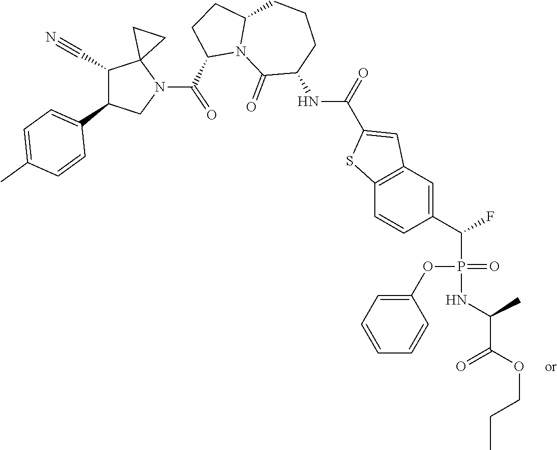 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6ccc(C)cc6)[C@H](C#N)C56CC6)N4C3=O)cc2c1] | 873.2 | ¹H NMR (400 MHz, DMSO) δ 8.99-7.72 (m, 8H), 7.68-7.07 (m, 6H), 6.32-6.05 (m, 2H), 4.99-4.90 (m, 1H), 4.55-4.49 (m, 1H), 4.25-4.18 (m, 1H), 3.94-3.80 (m, 2H), 3.77-3.44 (m, 3H), 3.20-3.01 (m, 1H), 2.31-1.51 (m, 15H), 1.30-1.12 (m, 4H), 1.01-0.64 (m, 9H), 0.58-0.32 (m, 2H) |
| | | 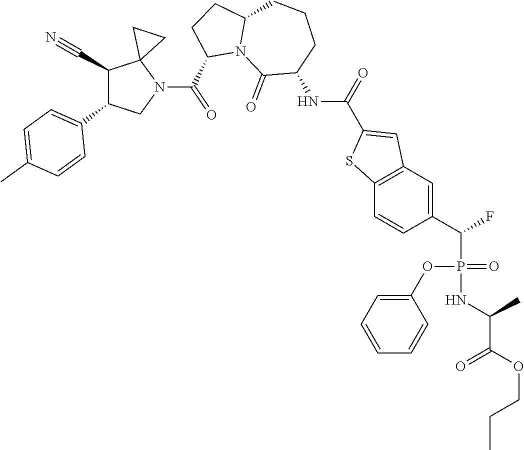 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6ccc(C)cc6)[C@@H](C#N)C56CC6)N4C3=O)cc2c1] | | |
| 478 | propyl (((R)-(2- (((3S,6S,9aS)-3- ((6R,7S)-7-cyano- 6-(p-tolyl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6S,7R)-7-cyano- 6-(p-tolyl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) | 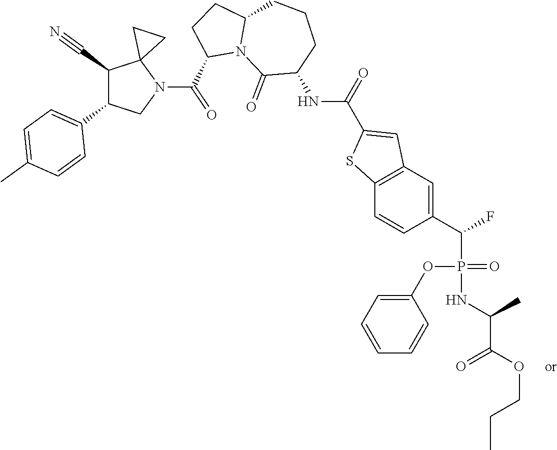 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@H](c6ccc(C)cc6)[C@@H](C#N)C56CC6)N4C3=O)cc2c1] | 868.6 | ¹H NMR (400 MHz, DMSO) δ 8.90-8.75 (m, 1H), 8.34-8.22 (m, 1H), 8.13-8.02 (m, 2H), 7.61 (t, J = 8.0 Hz, 1H), 7.42 7.29 (m, 4H), 7.24- 7.10 (m, 5H), 6.36- 6.02 (m, 2H), 4.71- 4.54 (m, 2H), 4.39- 4.19 (m, 1H), 4.05- 3.59 (m, 7H), 2.34- 2.26 (m, 3H), 2.23- 2.10 (m, 2H), 2.09- 1.89 (m, 2H), 1.88- 1.62 (m, 8H), 1.56- 1.39 (m, 2H), 1.19- 0.92 (m, 3H), 0.85- 0.75 (m, 4H), 0.73- 0.62 (m, 1H) |
| | (phenoxy)phosphoryl)- L-alaninate | 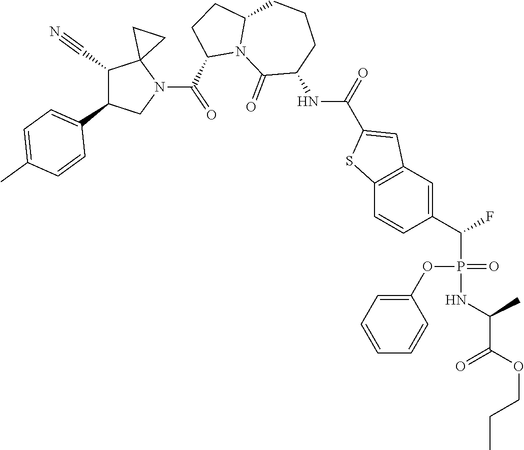 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6ccc(C)cc6)[C@H](C#N)C56CC6)N4C3=O)cc2c1] Pyrrolidine building block SFC peak 2 was used for synthesis and biological testing | | |
| 479 | propyl (((R)-(2- (((3S,6S,9aS)-3- ((6S,7R)-7-cyano- 6-(p-tolyl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate Or propyl (((R)-(2- (((3S,6S,9aS)-3- ((6R,7S)-7-cyano- 6-(p-tolyl)-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 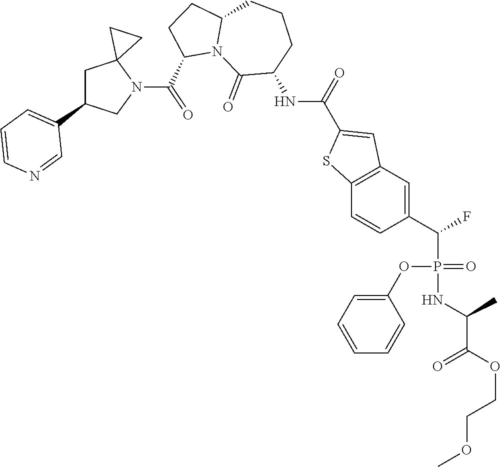 [SMILES: COCCOC(=O)[C@H](C)NP(=O)(Oc1ccccc1)[C@@H](F)c1ccc2sc(C(=O)N[C@H]3CCC[C@H]4CC[C@@H](C(=O)N5C[C@@H](c6cccnc6)CC56CC6)N4C3=O)cc2c1] Pyrrolidine building block SFC peak 1 was used for synthesis and biological testing | 868.2 | ¹H NMR (400 MHz, CDCl₃) δ 8.00-7.92 (m, 1H), 7.91-7.84 (m, 1H), 7.83-7.79 (m, 1H), 7.69-7.63 (m, 1H), 7.62-7.52 (m, 1H), 7.39-7.32 (m, 1H), 7.31-7.27 (m, 2H), 7.26-7.25 (m, 1H), 7.25-7.17 (m, 3H), 7.15-7.04 (m, 2H), 6.07-5.83 (m, 1H), 4.72-4.56 (m, 2H), 4.14-3.96 (m, 5H), 3.94-3.87 (m, 1H), 3.75-3.51 (m, 2H), 3.40-3.35 (m, 1H), 2.54-2.44 (m, 1H), 2.36 (s, 3H), 2.29-2.16 (m, 2H), 2.13-1.88 (m, 6H), 1.87-1.76 (m, 2H), 1.65-1.55 (m, 3H), 1.34-1.23 (m, 2H), 1.14-1.05 (m, 2H), 0.94-0.87 (m, 3H), 0.77-0.67 (m, 1H) |
| 480 | 2-methoxyethyl (((R)-fluoro(2- (((3S,6S,9aS)-5- oxo-3-((R)-6- (pyridin-3-yl)-4- azaspiro[2.4]heptane- 4- carbonyl)octahydro- 1H-pyrrolo[1,2- a]azepin-6- yl)carbamoyl)benzo [b]thiophen-5- yl)methyl)(phenoxy) phosphoryl)-L- alaninate | 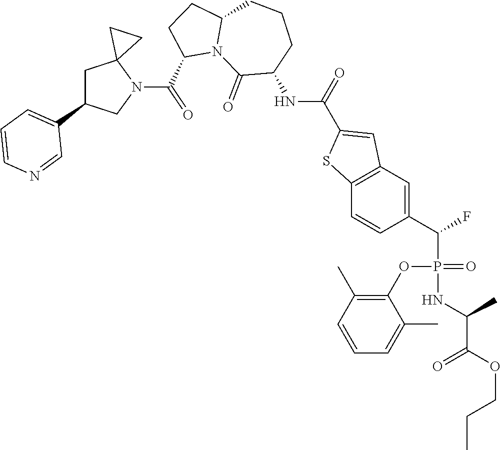 [SMILES: CCCOC(=O)[C@H](C)NP(=O)(Oc1c(C)cccc1C)[C@@H](F)c1ccc2sc(C(=O)N[C@H]3CCC[C@H]4CC[C@@H](C(=O)N5C[C@@H](c6cccnc6)CC56CC6)N4C3=O)cc2c1] | 846.2 | ¹H NMR (400 MHz, DMSO) δ 8.81-8.70 (m, 2H), 8.67-8.56 (m, 1H), 8.27 (s, 1H), 8.10-8.03 (m, 2H), 7.73-7.58 (m, 2H), 7.39-7.31 (m, 2H), 7.20-7.10 (m, 3H), 6.30-6.11 (m, 2H), 4.65-4.59 (m, 2H), 4.14-4.06 (m, 2H), 4.01-3.95 (m, 2H), 3.91-3.86 (m, 1H), 3.82-3.78 (m, 1H), 3.72-3.68 (m, 1H), 3.47-3.40 (m, 2H), 3.23-3.18 (m, 3H), 2.26-2.05 (m, 4H), 2.04-1.93 (m, 2H), 1.88-1.67 (m, 7H), 1.64-1.57 (m, 1H), 1.15 (d, J = 7.1 Hz, 3H), 0.56-0.37 (m, 2H) |
| 481 | propyl ((2,6- dimethylphenoxy) ((R)-fluoro(2- (((3S,6S,9aS)-5- oxo-3-((R)-6- (pyridin-3-yl)-4- azaspiro[2.4]heptane- 4- carbonyl)octahydro- 1H-pyrrolo[1,2- a]azepin-6- yl)carbamoyl)benzo [b]thiophen-5- yl)methyl)phosphoryl)- L-alaninate | 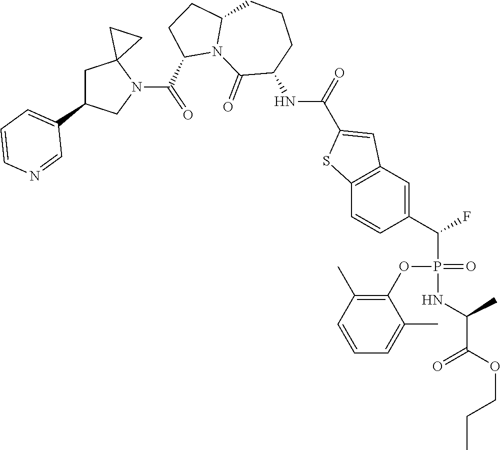 [SMILES: CCCOC(=O)[C@H](C)NP(=O)(Oc1c(C)cccc1C)[C@@H](F)c1ccc2sc(C(=O)N[C@H]3CCC[C@H]4CC[C@@H](C(=O)N5C[C@@H](c6cccnc6)CC56CC6)N4C3=O)cc2c1] | 858.2 | ¹H NMR (400 MHz, DMSO) δ 8.85-8.76 (m, 1H), 8.58 (s, 1H), 8.43 (d, J = 4.6 Hz, 1H), 8.27 (s, 1H), 8.12-8.03 (m, 2H), 7.79 (d, J = 7.9 Hz, 1H), 7.63 (d, J = 8.5 Hz, 1H), 7.36-7.29 (m, 1H), 7.06-6.94 (m, 3H), 6.31-6.14 (m, 1H), 5.97-5.72 (m, 1H), 4.67-4.58 (m, 2H), 4.10 (t, J = 8.5 Hz, 1H), 4.00- 3.66 (m, 5H), 3.62- 3.53 (m, 1H), 2.27- 2.15 (m, 8H), 2.13- 1.93 (m, 4H), 1.87- 1.67 (m, 7H), 1.62- 1.54 (m, 1H), 1.50- 1.34 (m, 2H), 1.19- 1.07 (m, 3H), 0.82- 0.72 (m, 3H), 0.53- 0.41 (m, 2H) |
| 482 | propyl (((R)-(2- (((3S,6S,10aS)-3- ((6R,7S)-7-cyano- 6-(3- (difluoromethyl) phenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6S,7R)-7-cyano- 6-(3- (difluoromethyl) phenyl)-4- azaspiro[2.4]heptane- 4-carbonyl)-5- oxodecahydropyrrolo | 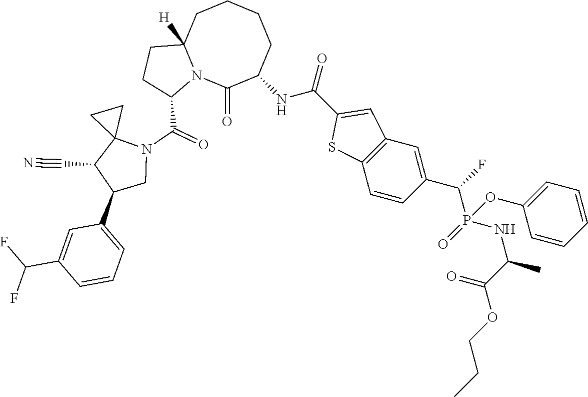 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cccc(C(F)F)c6)[C@H](C#N)C56CC6)N4C3=O)cc2c1] Or | 918.1 | ¹H NMR (400 MHz, CD₃OD) δ 8.13 (s, 1H), 8.08 (s, 1H), 8.00-7.92 (m, 1H), 7.67-7.48 (m, 5H), 7.38-7.30 (m, 2H), 7.26-7.11 (m, 3H), 6.76 (t, J = 56.0 Hz, 1H), 6.20-5.98 (m, 1H), 5.06-4.97 (m, 1H), 4.69-4.54 (m, 2H), 4.45-4.32 (m, 1H), 4.01-3.73 (m, 6H), 2.35-2.14 (m, 3H), 2.07-1.90 (m, 7H), 1.86-1.78 (m, 2H), 1.70-1.46 (m, 4H), 1.26-1.16 (m, 3H), 1.03-0.94 (m, 1H), 0.92-0.82 (m, 3H), 0.78-0.68 (m, 1H) |
| | [1,2-a]azocin-6- yl)carbamoyl)benzo [b]thiophen-5- yl)fluoromethyl) (phenoxy)phosphoryl)- L-alaninate | 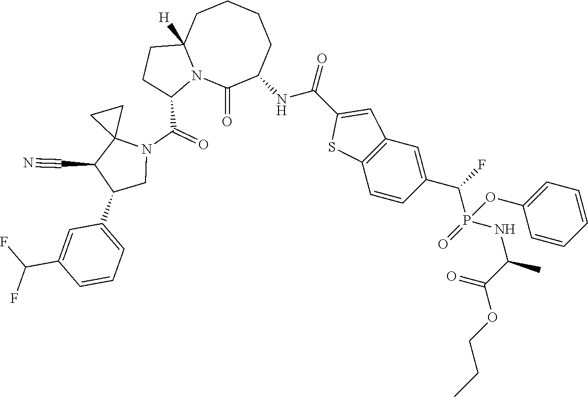 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccc(C(F)F)c6)[C@@H](C#N)C56CC6)N4C3=O)cc2c1] Pyrrolidine + Core SFC peak 1 was used for synthesis and biological testing | | |
| 483 | propyl (((R)-(2- (((3S,6S,10aS)-3- ((6R,7S)-7-cyano- 6-(3- (difluoromethyl) phenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate Or propyl (((R)-(2- (((3S,6S,10aS)-3- ((6S,7R)-7-cyano- 6-(3- (difluoromethyl) phenyl)-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 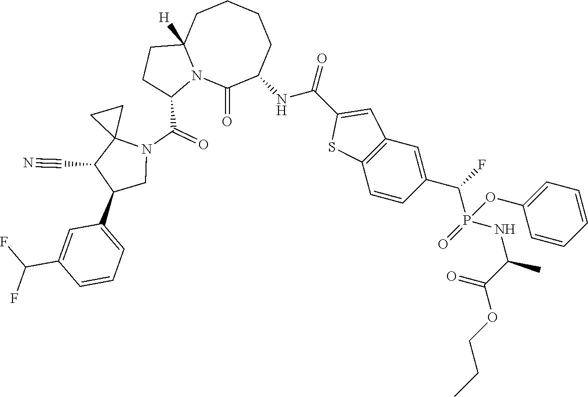 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@@H](c6cccc(C(F)F)c6)[C@H](C#N)C56CC6)N4C3=O)cc2c1] Or 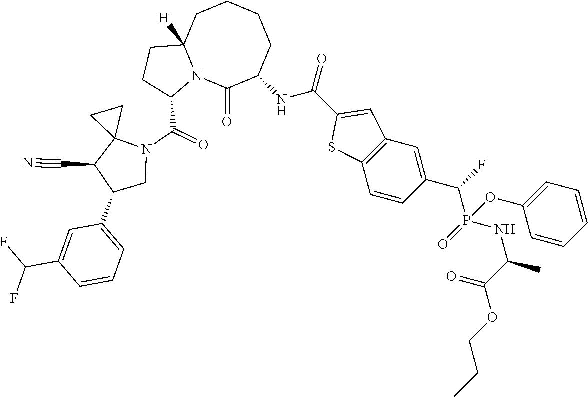 [SMILES: CCCOC(=O)[C@H](C)NP(=O)(Oc1ccccc1)[C@@H](F)c1ccc2sc(C(=O)N[C@H]3CCCC[C@H]4CC[C@@H](C(=O)N5C[C@H](c6cccc(C(F)F)c6)[C@@H](C#N)C56CC6)N4C3=O)cc2c1] Pyrrolidine + Core SFC peak 2 was used for synthesis and biological testing | 918.1 | ¹H NMR (400 MHz, CD₃OD) δ 8.13 (s, 1H), 8.08 (s, 1H), 8.01-7.97 (m, 1H), 7.70 (s, 1H), 7.67- 7.59 (m, 2H), 7.51- 7.47 (m, 2H), 7.37- 7.31 (m, 2H), 7.25- 7.13 (m, 3H), 6.76 (t, J = 56.2 Hz, 1H), 6.19-6.00 (m, 1H), 5.06-4.97 (m, 1H), 4.63-4.56 (m, 1H), 4.44-4.35 (m, 1H), 4.22-4.16 (m, 2H), 4.00-3.78 (m, 4H), 3.72-3.66 (m, 1H), 2.39-2.28 (m, 2H), 2.27-2.15 (m, 1H), 2.09-1.89 (m, 6H), 1.87-1.74 (m, 3H), 1.70-1.46 (m, 4H), 1.24-1.19 (m, 3H), 1.01-0.93 (m, 1H), 0.92-0.82 (m, 3H), 0.73-0.62 (m, 1H) |
| 484 | propyl (((S)-(2- (((3S,6S,9aS)-3-(3- (4-ethoxypyridin-3- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 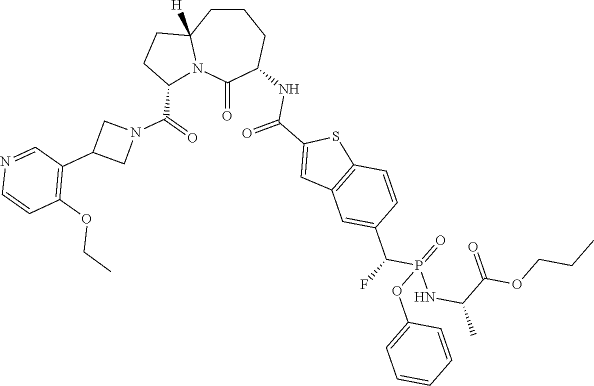 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cnccc6OCC)C5)N4C3=O)cc2c1] | 834.1 | ¹H NMR (400 MHz, CD₃OD) δ 8.40- 8.32 (m, 1H), 8.14- 8.05 (m, 2H), 8.04- 7.93 (m, 1H), 7.70- 7.59 (m, 1H), 7.39- 7.28 (m, 2H), 7.26- 7.14 (m, 3H), 7.05 (dd, J = 19.0, 5.5 Hz, 1H), 6.19-5.99 (m, 1H), 4.80-4.71 (m, 1H), 4.70-4.62 (m, 1H), 4.62-4.57 (m, 1H), 4.49-4.31 (m, 2H), 4.25-4.16 (m, 3H), 4.15-4.03 (m, 2H), 3.99-3.95 (m, 1H), 3.94-3.85 (m, 1H), 3.85-3.75 (m, 1H), 2.37-2.26 (m, 1H), 2.17-2.04 (m, 2H), 2.03-1.81 (m, 7H), 1.60-1.51 (m, 2H), 1.45 (td, J = 7.0, 1.5 Hz, 3H), 1.23 (dd, J = 7.0, 1.8 Hz, 2H), 1.07 (dd, J = 7.0, 3.8 Hz, 2H), 0.91- 0.84 (m, 3H) |
| 485 | propyl (((S)-(2- (((3S,6S,10aS)-3- ((6S,7R)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 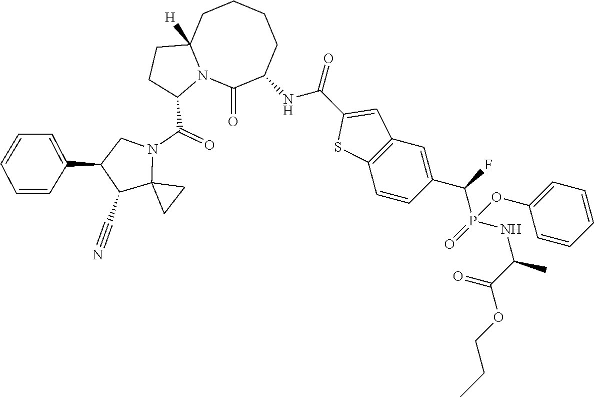 [SMILES: CCCOC(=O)[C@H](C)NP(=O)(Oc1ccccc1)[C@H](F)c1ccc2sc(C(=O)N[C@H]3CCCC[C@H]4CC[C@@H](C(=O)N5C[C@H](c6ccccc6)[C@@H](C#N)C56CC6)N4C3=O)cc2c1] | 868.1 | ¹H NMR (400 MHz, CD₃OD) δ 8.12 (s, 1H), 8.09-8.05 (m, 1H), 7.97 and 7.94 (d, J = 8.6 Hz, 1H), 7.67-7.59 (m, 1H), 7.44-7.28 (m, 7H), 7.24-7.14 (m, 3H), 6.13 and 6.02 (dd, J = 17.6, 6.4 Hz, 1H), 5.07-4.96 (m, 1H), 4.68-4.51 (m, 2H), 4.44-4.32 (m, 1H), 4.02-3.68 (m, 6H), 2.36-2.13 (m, 3H), 2.08-1.86 (m, 7H), 1.85-1.74 (m, 2H), 1.70-1.47 (m, 4H), 1.23 and 1.06 (d, J = 7.1 Hz, 3H), 1.02-0.93 (m, 1H), 0.91-0.82 (m, 3H), 0.78-0.65 (m, 1H) |
| 486 | propyl (((2- (((3S,6S,9aS)-3- ((6R,7S)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 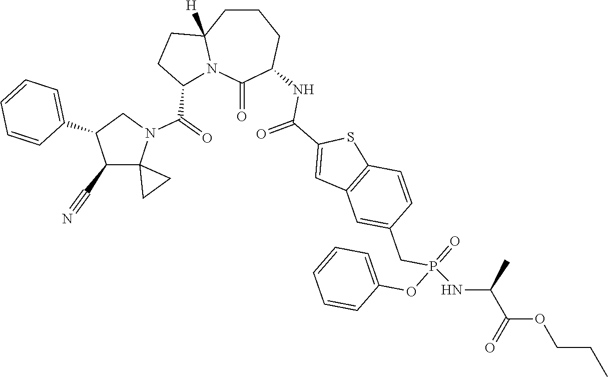 [SMILES: CCCOC(=O)[C@H](C)NP(=O)(Cc1ccc2sc(C(=O)N[C@H]3CCC[C@H]4CC[C@@H](C(=O)N5C[C@@H](c6ccccc6)[C@H](C#N)C56CC6)N4C3=O)cc2c1)Oc1ccccc1] | 836.1 | ¹H NMR (400 MHz, CD₃OD) δ 8.04 (s, 1H), 7.96-7.84 (m, 2H), 7.55-7.44 (m, 3H), 7.43-7.25 (m, 5H), 7.24-7.10 (m, 3H), 4.78-4.63 (m, 2H), 4.20-4.10 (m, 1H), 4.09-3.99 (m, 2H), 3.98-3.68 (m, 5H), 3.61-3.43 (m, 2H), 2.38-1.75 (m, 12H), 1.60-1.48 (m, 2H), 1.14 (d, J = 7.3 Hz, 1.5H), 1.08 (d, J = 7.1 Hz, 1.5H), 1.04-0.95 (m, 1H), 0.87 (t, J = 7.5 Hz, 1.5H), 0.86 (t, J = 7.5 Hz, 1.5H), 0.76-0.64 (m, 1H). |
| 487 | propyl (((S)-(2- (((3S,6S,9aS)-3- ((6S,7R)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-prolinate | 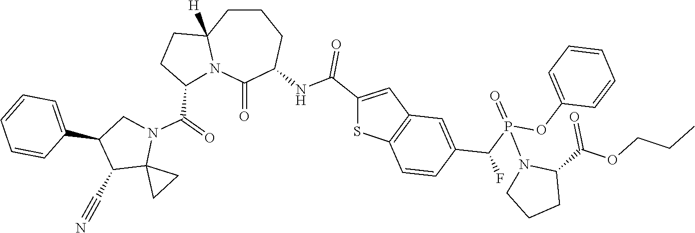 [SMILES: CCCOC(=O)[C@@H]1CCCN1P(=O)(Oc1ccccc1)[C@H](F)c1ccc2sc(C(=O)N[C@H]3CCC[C@H]4CC[C@@H](C(=O)N5C[C@H](c6ccccc6)[C@@H](C#N)C56CC6)N4C3=O)cc2c1] | 880.1 | ¹H NMR (400 MHz, CD₃OD) δ 8.17- 7.97 (m, 3H), 7.73- 7.59 (m, 1H), 7.48- 7.17 (m, 10H), 6.36- 6.12 (m, 1H), 4.77- 4.62 (m, 2H), 4.48- 4.46 (m, 1H), 4.10- 3.92 (m, 4H), 3.89- 3.71 (m, 3H), 3.67- 3.39 (m, 2H), 3.22- 3.08 (m, 1H), 2.37- 2.22 (m, 2H), 2.17- 2.06 (m, 2H), 2.02- 1.76 (m, 11H), 1.66- 1.54 (m, 2H), 1.06- 0.98 (m, 1H), 0.94- 0.87 (m, 3H), 0.80- 0.70 (m, 1H) |
| 488 | propyl (2S)-1-(((S)- (2-(((3S,6S,9aS)-3- ((6S,7R)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)fluoromethyl) (phenoxy)phosphoryl) azetidine-2- carboxylate | 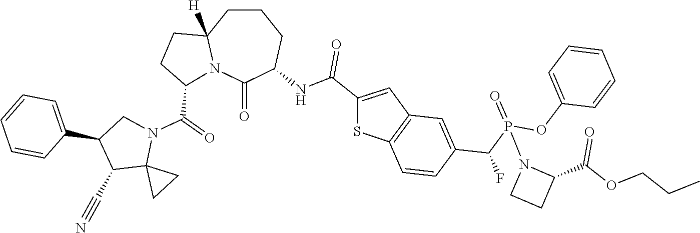 [SMILES: CCCOC(=O)[C@@H]1CCN1P(=O)(Oc1ccccc1)[C@H](F)c1ccc2sc(C(=O)N[C@H]3CCC[C@H]4CC[C@@H](C(=O)N5C[C@H](c6ccccc6)[C@@H](C#N)C56CC6)N4C3=O)cc2c1] | 866.1 | ¹H NMR (400 MHz, CD₃OD) δ 8.21- 8.00 (m, 3H), 7.76- 7.64 (m, 1H), 7.47- 7.20 (m, 10H), 6.43- 6.25 (m, 1H), 4.74- 4.71 (m, 2H), 4.58- 4.54 (m, 1H), 4.47- 4.44 (m, 1H), 4.18- 4.10 (m, 2H), 4.07- 3.88 (m, 2H), 3.87- 3.71 (m, 3H), 3.51- 3.42 (m, 1H), 2.66- 2.56 (m, 1H), 2.38- 2.23 (m, 3H), 2.17- 2.05 (m, 2H), 2.02- 1.79 (m, 8H), 1.71- 1.61 (m, 2H), 1.05- 0.98 (m, 1H), 0.95 (t, J = 7.5 Hz, 3H), 0.78- 0.68 (m, 1H) |
| 489 | propyl (((R)-(2- (((3S,6S,9S,10aR)- 9-(azetidin-1-yl)-3- ((6R,7S)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 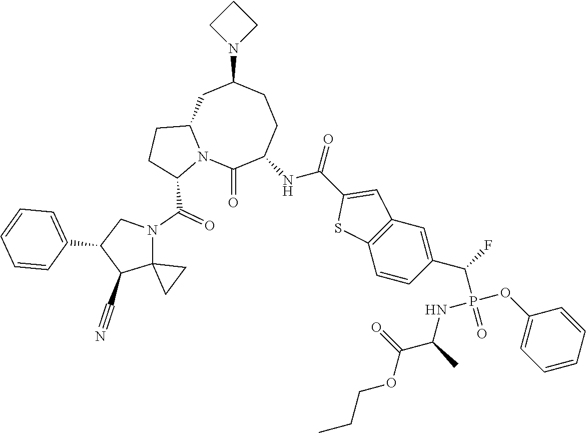 [SMILES: CCCOC(=O)[C@H](C)NP(=O)(Oc1ccccc1)[C@@H](F)c1ccc2sc(C(=O)N[C@H]3CC[C@H](N4CCC4)C[C@H]4CC[C@@H](C(=O)N5C[C@@H](c6ccccc6)[C@H](C#N)C56CC6)N4C3=O)cc2c1] | 923.2 | ¹H NMR (400 MHz, CD₃OD) δ 8.14- 8.06 (m, 2H), 8.00- 7.95 (m, 1H), 7.66- 7.62 (m, 1H), 7.49- 7.15 (m, 10H), 6.23- 5.95 (m, 1H), 4.97- 4.93 (m, 1H), 4.66- 4.44 (m, 3H), 4.02- 3.62 (m, 6H), 3.55- 3.45 (m, 2H), 2.85- 2.75 (m, 1H), 2.43- 2.31 (m, 2H), 2.20- 1.78 (m, 11H), 1.60- 1.49 (m, 2H), 1.38- 1.28 (m, 5H), 1.24- 1.19 (m, 2H), 0.90- 0.84 (m, 3H), 0.75- 0.65 (m, 1H) |
| 490 | propyl (((R)-(2- (((3S,6S,9S,10aR)- 9-(azetidin-1-yl)-3- ((6S,7R)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 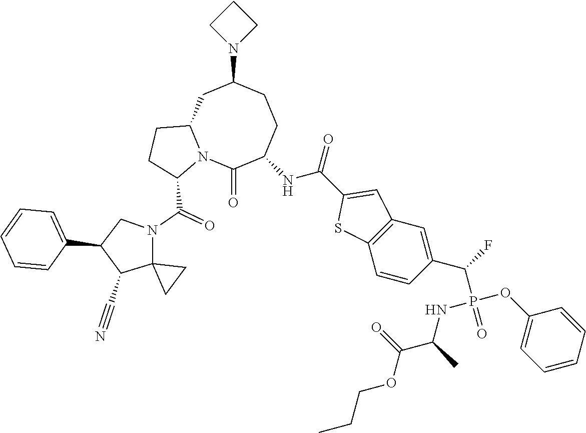 [SMILES: CCCOC(=O)[C@H](C)NP(=O)(Oc1ccccc1)[C@@H](F)c1ccc2sc(C(=O)N[C@H]3CC[C@H](N4CCC4)C[C@H]4CC[C@@H](C(=O)N5C[C@H](c6ccccc6)[C@@H](C#N)C56CC6)N4C3=O)cc2c1] | 923.2 | ¹H NMR (400 MHz, CD₃OD) δ 8.14- 8.06 (m, 2H), 8.03- 7.92 (m, 1H), 7.69- 7.58 (m, 1H), 7.46- 7.28 (m, 7H), 7.25- 7.11 (m, 3H), 6.19- 5.95 (m, 1H), 4.99- 4.89 (m, 1H), 4.70- 4.60 (m, 1H), 4.58- 4.43 (m, 2H), 4.02- 3.68 (m, 6H), 3.54- 3.42 (m, 2H), 2.91- 2.74 (m, 1H), 2.42- 2.28 (m, 2H), 2.22- 1.77 (m, 11H), 1.61- 1.48 (m, 2H), 1.37- 1.30 (m, 2H), 1.25- 1.19 (m, 3H), 1.06- 0.96 (m, 1H), 0.94- 0.83 (m, 4H), 0.78- 0.69 (m, 1H) |
| 491 | propyl (((S)-(2- (((3S,6S,9S,10aR)- 9-(azetidin-1-yl)-3- ((6S,7R)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 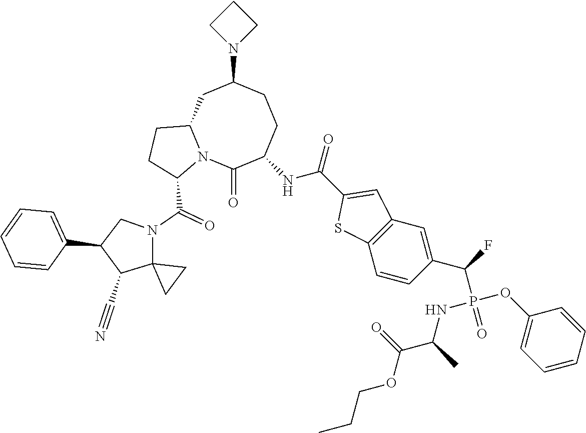 [SMILES: CCCOC(=O)[C@H](C)NP(=O)(Oc1ccccc1)[C@H](F)c1ccc2sc(C(=O)N[C@H]3CC[C@H](N4CCC4)C[C@H]4CC[C@@H](C(=O)N5C[C@H](c6ccccc6)[C@@H](C#N)C56CC6)N4C3=O)cc2c1] | 923.2 | ¹H NMR (400 MHz, CD₃OD) δ 8.14- 8.05 (m, 2H), 8.02- 7.91 (m, 1H), 7.68- 7.60 (m, 1H), 7.44- 7.30 (m, 7H), 7.26- 7.11 (m, 3H), 6.19- 5.98 (m, 1H), 4.97- 4.90 (m, 1H), 4.69- 4.61 (m, 1H), 4.58- 4.44 (m, 2H), 4.03- 3.69 (m, 6H), 3.52- 3.41 (m, 2H), 2.87- 2.76 (m, 1H), 2.43- 2.30 (m, 2H), 2.22- 1.75 (m, 11H), 1.63- 1.48 (m, 2H), 1.43- 1.16 (m, 5H), 1.09- 0.96 (m, 2H), 0.91- 0.83 (m, 3H), 0.78- 0.69 (m, 1H) |
| 492 | propyl (((S)-(2- (((3S,6S,9S,10aR)- 9-(azetidin-1-yl)-3- ((6R,7S)-7-cyano- 6-phenyl-4- azaspiro[2.4]heptane- 4-carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 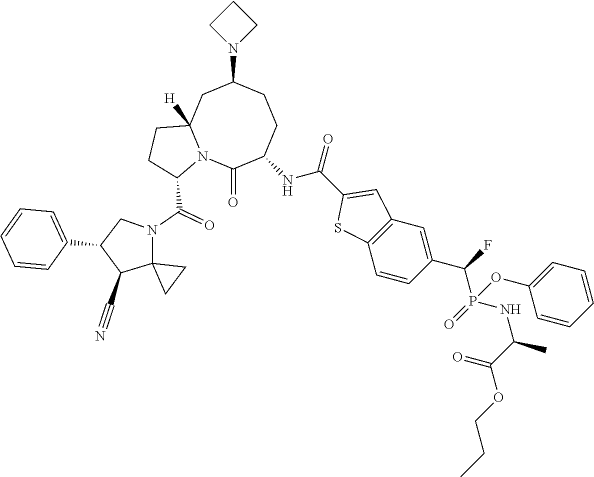 [SMILES: CCCOC(=O)[C@H](C)NP(=O)(Oc1ccccc1)[C@H](F)c1ccc2sc(C(=O)N[C@H]3CC[C@H](N4CCC4)C[C@H]4CC[C@@H](C(=O)N5C[C@@H](c6ccccc6)[C@H](C#N)C56CC6)N4C3=O)cc2c1] | 923.2 | ¹H NMR (400 MHz, CD₃OD) δ 8.11- 8.05 (m, 2H), 7.99- 7.94 (m, 1H), 7.68- 7.61 (m, 1H), 7.48- 7.46 (m, 2H), 7.37- 7.25 (m, 5H), 7.24- 7.14 (m, 3H), 6.18- 5.98 (m, 1H), 4.96- 4.92 (m, 1H), 4.61- 4.44 (m, 2H), 4.19- 4.08 (m, 2H), 4.01- 3.87 (m, 2H), 3.85- 3.65 (m, 3H), 3.50- 3.37 (m, 2H), 3.28- 3.20 (m, 2H), 2.80- 2.70 (m, 1H), 2.45- 2.33 (m, 2H), 2.21- 2.10 (m, 2H), 2.10- 1.91 (m, 4H), 1.90- 1.72 (m, 5H), 1.60- 1.48 (m, 2H), 1.37- 1.26 (m, 2H), 1.25- 1.21 (m, 1H), 1.07- 1.05 (m, 1H), 1.02- 0.94 (m, 1H), 0.92- 0.82 (m, 3H), 0.76- 0.67 (m, 1H) |
| 493 | propyl (((R)-(2- (((3S,6S,9R,10aR)- 3-(3-(4-(azetidin-1- yl)pyridin-3- yl)azetidine-1- carbonyl)-9-ethyl- 5- oxodecahydropyrrolo [1,2-a]azocin-6- yl)carbamoyl)benzo [b]thiophen-5- yl)fluoromethyl) (phenoxy)phosphoryl)- L-alaninate | 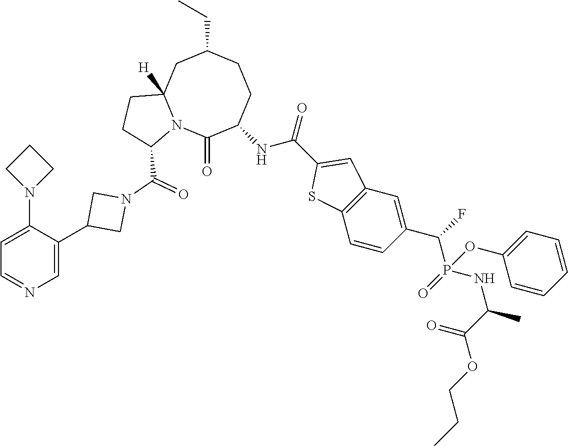 [SMILES: CCCOC(=O)[C@H](C)NP(=O)(Oc1ccccc1)[C@@H](F)c1ccc2sc(C(=O)N[C@H]3CC[C@@H](CC)C[C@H]4CC[C@@H](C(=O)N5CC(c6cnccc6N6CCC6)C5)N4C3=O)cc2c1] | 887.2 | ¹H NMR (400 MHz, CD₃OD) δ 8.28- 8.21 (m, 1H), 8.13- 7.84 (m, 4H), 7.67- 7.57 (m, 1H), 7.39- 7.29 (m, 2H), 7.27- 7.10 (m, 3H), 6.42- 5.97 (m, 2H), 5.05- 4.94 (m, 1H), 4.74- 4.59 (m, 1H), 4.54- 4.14 (m, 4H), 4.07- 3.80 (m, 8H), 2.33- 2.18 (m, 4H), 2.12- 1.78 (m, 6H), 1.69- 1.47 (m, 5H), 1.44- 1.28 (m, 3H), 1.26- 1.18 (m, 3H), 0.98- 0.95 (m, 3H), 0.92- 0.81 (m, 3H) |
| 494 | propyl (((R)-(2- (((3S,6S,9aS)-3-(3- (4-ethoxypyridin-3- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy)phosphoryl)- L-alaninate | 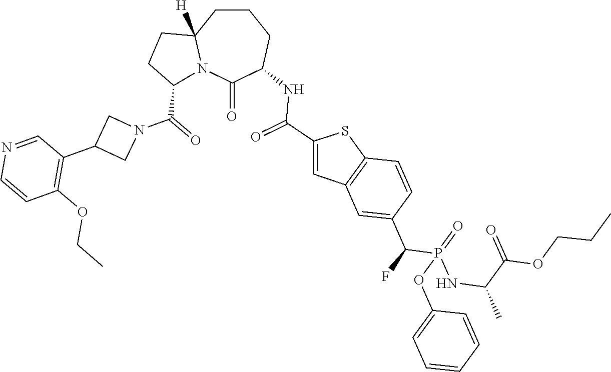 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cnccc6OCC)C5)N4C3=O)cc2c1] | 834.4 | ¹H NMR (400 MHz, CD₃OD) δ 8.38- 8.27 (m, 2H), 8.14- 8.05 (m, 2H), 8.02- 7.95 (m, 1H), 7.69- 7.61 (m, 1H), 7.38- 7.30 (m, 2H), 7.25- 7.14 (m, 3H), 7.09- 6.99 (m, 1H), 6.20- 5.97 (m, 1H), 4.84- 4.80 (m, 1H), 4.79- 4.71 (m, 1H), 4.69- 4.55 (m, 2H), 4.49- 4.30 (m, 1H), 4.27- 4.15 (m, 3H), 4.15- 4.03 (m, 2H), 4.01- 3.95 (m, 1H), 3.94- 3.78 (m, 2H), 2.37- 2.26 (m, 1H), 2.19- 1.76 (m, 9H), 1.61- 1.47 (m, 2H), 1.44 (td, J = 7.0, 2.4 Hz, 3H), 1.24-1.19 (m, 3H), 0.92-0.82 (m, 3H) |

### Compounds Prepared According to General Scheme 2: Phosphonic Acids

The following compounds in Table 109 were prepared according to the representative procedure described above for the synthesis of (difluoro(2-(((3S,6S,10aR,Z)-3-(methyl(phenyl)carbamoyl)-5-oxo-1,2,3,5,6,7,10,10a-octahydropyrrolo[1,2-a]azocin-6-yl)carbamoyl)-1H-indol-5-yl)methyl)phosphonic acid (30) utilizing appropriate starting materials and modifications.

TABLE 109

| | | | LCMS | |
|---|---|---|---|---|
| Compound | Name | Structure | [M + H]⁺ | ¹H NMR |
| 495 | (difluoro(2- (((3S,6S,10aS)-3-(3- (1-hydroxyethyl)-3- phenylazetidine-1- carbonyl)-5- oxodecahydro- pyrrolo[1,2-a] azocin-6- yl)carbamoyl)benzo | 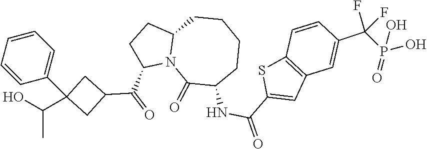 [SMILES: CC(O)C1(c2ccccc2)CC(C(=O)[C@@H]2CC[C@@H]3CCCC[C@H](NC(=O)c4cc5cc(C(F)(F)P(=O)(O)O)ccc5s4)C(=O)N32)C1] | 676.3 | ¹H NMR (400 MHz, CD₃OD) δ 8.14-8.19 (m, 1H) 7.92-8.11 (m, 3H) 7.68 (s, 1H) 7.35 (d, J=8.0 Hz, 1H) 7.27-7.30 (m, 1H) 7.19-7.22 (m, 1H) 7.13-7.16 (m, 1H) 4.94-5.06 (m, |
| | [b]thiophen-5- | | | 1H) 4.33-4.60 (m, |
| | yl)methyl) | | | 4H) 4.22-4.30 (m, |
| | phosphonic acid | | | 1H) 4.14 (s, 1H) .94- |
| | | | | 4.04 (m, 1H) 2.18- |
| | | | | 2.32 (m, 2H) 1.91- |
| | | | | 2.07 (m, 6H) 1.83 (m, |
| | | | | 2H) 1.60-1.71 (m, |
| | | | | 2H) 0.91-0.95 (m, |
| | | | | 3H) |
| 496 | ((2-(((3S,6S,10aS)- 3-(3-(5-cyano-3- methylpyridin-2- yl)azetidine-1- carbonyl)-5- oxodecahydro- pyrrolo[1,2- a]azocin-6- yl)carbamoyl)benzo [b]thiophen-5- yl)difluoromethyl) | 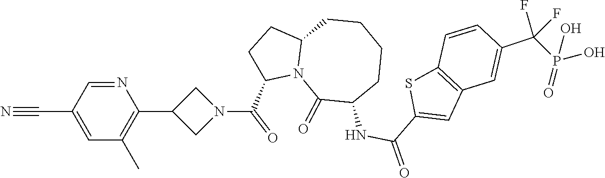 [SMILES: Cc1cc(C#N)cnc1C1CN(C(=O)[C@@H]2CC[C@@H]3CCCC[C@H](NC(=O)c4cc5cc(C(F)(F)P(=O)(O)O)ccc5s4)C(=O)N32)C1] | 672.1 | ¹H NMR (400 MHz, CD₃OD) δ 8.79-8.62 (m, 1H), 8.25-8.19 (m, 1H), 8.06-7.92 (m, 2H), 7.90 (d, J = 7.6 Hz, 1H), 7.71- 7.60 (m, 1H), 5.06- 4.91 (m, 1H), 4.81- 4.74 (m, 1H), 4.67- 4.53 (m, 1H), 4.52- 4.37 (m, 3H), 4.34- |
| | phosphonic acid | | | 4.21 (m, 2H), 2.34- |
| | | | | 2.15 (m, 5H), 2.09- |
| | | | | 1.89 (m, 6H), 1.88- |
| | | | | 1.75 (m, 2H), 1.73- |
| | | | | 1.58 (m, 2H) |
| 497 | ((2-(((3S,6S,10aS)- 3-(3- (benzo[d]oxazol-2- yl)azetidine-1- carbonyl)-5- oxodecahydro- pyrrolo[1,2- a]azocin-6- yl)carbamoyl)benzo [b]thiophen-5- yl)difluoromethyl) | 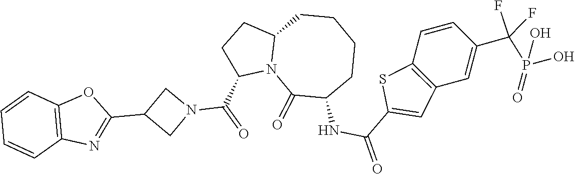 [SMILES: O=C(N[C@H]1CCCC[C@H]2CC[C@@H](C(=O)N3CC(c4nc5ccccc5o4)C3)N2C1=O)c1cc2cc(C(F)(F)P(=O)(O)O)ccc2s1] | 673.2 | ¹H NMR (400 MHz, CD₃OD) δ 8.21-8.04 (m, 2H), 7.96-7.88 (m, 1H), 7.77-7.71 (m, 1H), 7.70-7.56 (m, 2H), 7.42-7.32 (m, 2H), 5.01 (t, J = 8.4 Hz, 1H), 4.75- 4.69 (m, 1H), 4.62- 4.55 (m, 1H), 4.50- 4.38 (m, 3H), 4.32- |
| | phosphonic acid | | | 4.19 (m, 2H), 2.32- |
| | | | | 2.20 (m, 2H), 2.07- |
| | | | | 1.92 (m, 6H), 1.89- |
| | | | | 1.78 (m, 2H), 1.73- |
| | | | | 1.58 (m, 2H) |
| 498 | (difluoro(2- (((3S,6S,9aS)-3-(3- methoxy-3-(pyridin- 3-yl)azetidine-1- carbonyl)-5- oxooctahydro-1H- pyrrolo[1,2- a]azepin-6- yl)carbamoyl)benzo [b]thiophen-5- | 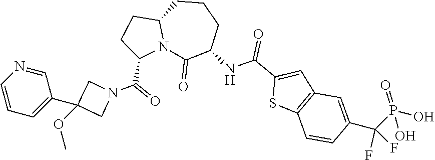 | 649.2 | ¹H NMR (400 MHz, CD₃OD) δ 8.72-8.67 (m, 1H), 8.56-8.52 (m, 1H), 8.20 (d, J = 6.0 Hz, 1H), 8.14- 8.07 (m, 1H), 8.04- 7.97 (m, 1H), 7.96- 7.90 (m, 1H), 7.75 (d, J = 8.4 Hz, 1H), 7.56- 7.45 (m, 1H), 4.82- |
| | yl)methyl) | | | 4.70 (m, 2H), 4.65- |
| | phosphonic acid | | | 4.52 (m, 2H), 4.38- |
| | | | | 4.20 (m, 2H), 4.07 (d, |
| | | | | J = 4.4 Hz, 1H), 3.20- |
| | | | | 3.13 (m, 3H), 2.39- |
| | | | | 2.25 (m, 1H), 2.20 (s, |
| | | | | 1H), 2.11-2.02 (m, |
| | | | | 2H), 1.98-1.91 (m, |
| | | | | 3H), 1.88-1.77 (s, |
| | | | | 3H |
| 499 | (difluoro(2- (((3S,6S,10aS)-3-(3- (4-methylpyridin-3- yl)azetidine-1- carbonyl)-5- oxodecahydropyrrol o[1,2-a]azocin-6- yl)carbamoyl)benzo [b]thiophen-5- | 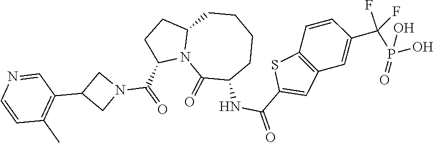 [SMILES: Cc1ccncc1C1CN(C(=O)[C@@H]2CC[C@@H]3CCCC[C@H](NC(=O)c4cc5cc(C(F)(F)P(=O)(O)O)ccc5s4)C(=O)N32)C1] | 647.0 | ¹H NMR (400 MHz, DMSO-d₆) δ 8.66- 8.51 (m, 1H), 8.44- 8.34 (m, 1H), 8.30 (m, 1H), 8.23-8.01 (m, 1H), 7.67-7.55 (m, 1H), 7.40-7.30 (m, 2H), 5.04-4.86 (m, 2H), 4.74-4.72 |
| | yl)methyl) | | | (m, 1H), 4.71-4.47 |
| | phosphonic acid | | | (m, 3H), 4.45-4.44 |
| | | | | (m, 1H), 4.42-4.17 |
| | | | | (m, 1H), 2.47-2.27 |
| | | | | (m, 3H), 2.25-2.23 |
| | | | | (m, 2H), 2.05-2.03 |
| | | | | (m, 6H), 2.02-1.99 |
| | | | | (m, 2H), 1.98-1.68 |
| | | | | (m, 2H) |
| 500 | ((2-(((3S,6S,9aR)-8- methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) | 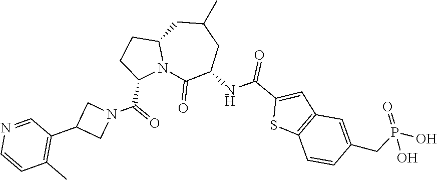 | 611.1 | ¹H NMR (400 MHz, CD₃OD) δ 8.79 (s, 1H), 8.62-8.44 (m, 1H), 8.05-7.91 (m, 1H), 7.89-7.74 (m, 3H), 7.47-7.37 (m, 1H), 4.80-4.71 (m, 2H), 4.69-4.58 (m, 1H), 4.58-4.51 (m, 1H), 4.49-4.39 (m, 1H), 4.38-4.26 (m, |
| | phosphonic acid | | | 1H), 4.20-4.04 (m, |
| | | | | 2H), 3.28-3.18 (m, |
| | | | | 2H), 2.54 (s, 3H), |
| | | | | 2.38-2.28 (m, 1H), |
| | | | | 2.19-1.91 (m, 5H), |
| | | | | 1.80 (d, J = 11.2 Hz, |
| | | | | 1H), 1.69-1.50 (m, |
| | | | | 2H), 1.05 (d, J = 6.4 |
| | | | | Hz, 3H) |
| 501 | (difluoro(2- (((3S,6S,10aS)-3-(3- methoxy-3-(4- methylpyridin-3- yl)azetidine-1- carbonyl)-5- oxodecahydro- pyrrolo[1,2-a] azocin-6- yl)carbamoyl)benzo | 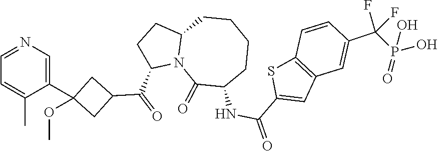 [SMILES: COC1(c2cnccc2C)CC(C(=O)[C@@H]2CC[C@@H]3CCCC[C@H](NC(=O)c4cc5cc(C(F)(F)P(=O)(O)O)ccc5s4)C(=O)N32)C1] | 677.2 | 1H NMR (400 MHz, DMSO-d₆) δ 8.97- 8.81 (m, 1H), 8.76- 8.53 (m, 2H), 8.38- 8.21 (m, 1H), 8.16- 8.01 (m, 2H), 7.66- 7.45 (m, 2H), 5.02- 4.81 (m, 2H), 4.72 (d, J = 5.2 Hz, 1H), 4.58- 4.38 (m, 2H), 4.37- |
| | [b]thiophen-5- | | | 4.06 (m, 4H), 2.93 (d, |
| | yl)methyl) | | | J = 10.4 Hz, 3H), 2.36 |
| | phosphonic acid | | | (d, J = 13.2 Hz, 3H), |
| | | | | 2.26-2.20 (m, 1H), |
| | | | | 1.89-1.70 (m, 8H), |
| | | | | 1.65-1.54 (m, 2H), |
| | | | | 1.52-1.40 (m, 1H) |
| 502 | ((2-(((3S,6S,9aS)-3- (3-(4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 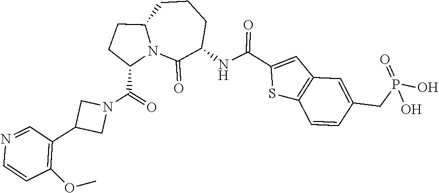 | 613.0 | ¹H NMR (400 MHz, CD₃OD) δ 8.52-8.37 (m, 2H), 7.99-7.87 (m, 1H), 7.81-7.70 (m, 2H), 7.51-7.36 (m, 2H), 5.03 (d, J = 8.4 Hz, 1H), 4.85- 4.84 (m, 1H), 4.87- 4.70 (m, 1H), 4.65- 4.50 (m, 2H), 4.37- 4.30 (m, 1H), 4.15- 4.01 (m, 5H), 3.25- |
| | | | | 3.13 (m, 2H), 2.37- |
| | | | | 2.26 (m, 1H), 2.20- |
| | | | | 2.06 (m, 2H), 2.04- |
| | | | | 1.77 (m, 7H) |
| 503 | ((2-(((3S,6S,9aS)-3- (3-(4-ethylpyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 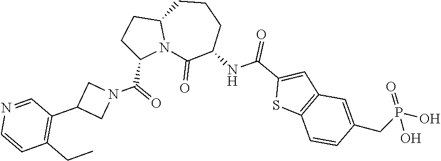 | 611.3 | ¹H NMR (400 MHz, CD₃OD) δ 8.61-8.44 (m, 1H), 8.29-8.10 (m, 1H), 7.95-7.62 (m, 3H), 7.47-7.23 (m, 2H), 4.96-4.91 (m, 1H), 4.73 (d, J = 10.6 Hz, 1H), 4.70- 4.63 (m, 1H), 4.61- 4.53 (m, 1H), 4.42- 4.28 (m, 1H), 4.26- |
| | | | | 4.13 (m, 1H), 4.13- |
| | | | | 4.03 (m, 2H), 3.20- |
| | | | | 3.05 (m, 2H), 2.69- |
| | | | | 2.56 (m, 2H), 2.37- |
| | | | | 2.27 (m, 1H), 2.20- |
| | | | | 2.07 (m, 2H), 2.05- |
| | | | | 1.94 (m, 3H), 1.93- |
| | | | | 1.76 (m, 4H), 1.22- |
| | | | | 1.14 (m, 3H) |
| 504 | ((2-(((3S,6S,9aS)-3- (3-(5-fluoro-4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) | 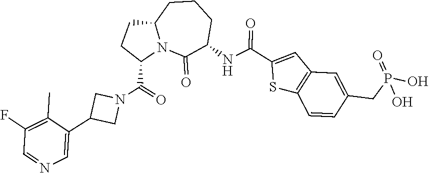 | 615.1 | ¹H NMR (400 MHz, CD₃OD) δ 8.51-8.40 (m, 1H), 8.32-8.23 (m, 1H), 7.98 (d, J = 11.2 Hz, 1H), 7.86- 7.73 (m, 2H), 7.44 (t, J = 7.6 Hz, 1H), 4.99- 4.92 (m, 1H), 4.77- 4.71 (m, 1H), 4.71- 4.66 (m, 1H), 4.61- 4.55 (m, 1H), 4.54- |
| | phosphonic acid | | | 4.38 (m, 1H), 4.27- |
| | | | | 4.19 (m, 1H), 4.18- |
| | | | | 4.03 (m, 2H), 3.17- |
| | | | | 3.07 (m, 2H), 2.34- |
| | | | | 2.27 (m, 1H), 2.23 (s, |
| | | | | 3H), 2.18-2.06 (m, |
| | | | | 2H), 2.06-1.95 (m, |
| | | | | 3H), 1.93-1.74 (m, |
| | | | | 4H |
| 505 | ((2-(((3S,6S,9aS)-3- (3-(2-fluoro-4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) | 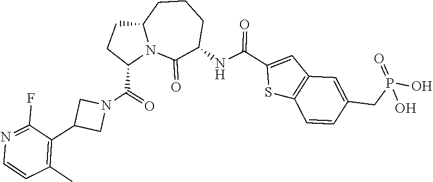 | 615.0 | ¹H NMR (400 MHz, CD₃OD) δ 8.02 (s, 1H) 7.91-7.97 (m, 1H) 7.84 (s, 2H) 7.40- 7.46 (m, 1H) 7.11- 7.17 (m, 1H) 4.66 (m, 2H) 4.55-4.63 (m, 1H) 4.40-4.48 (m, 1H) 4.26-4.38 (m, 2H) 4.13-4.22 (m, 1H) 4.01-4.12 (m, |
| | phosphonic acid | | | 1H) 3.28 (s, 1H) 3.21- |
| | | | | 3.24 (m, 1H) 2.40 |
| | | | | (d, J = 4.0 Hz, 3H) |
| | | | | 2.31 (s, 1H) 2.14- |
| | | | | 2.20 (m, 1H) 1.82- |
| | | | | 2.08 (m, 8H) |
| 506 | ((2-(((3S,6S,9aS)-3- (3-(5-methoxy-4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 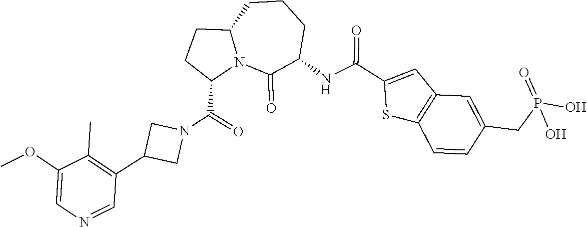 | 627.3 | ¹H NMR (400 MHz, CD₃OD) δ 8.45 (s, 1H), 8.34-8.24 (m, 1H), 8.01-7.91 (m, 1H), 7.85-7.76 (m, 2H), 7.46-7.37 (m, 1H), 4.78-4.71 (m, 2H), 4.67-4.57 (m, 1H), 4.56-4.50 (m, 1H), 4.48-4.38 (m, 1H), 4.37-4.28 (m, 1H), 4.18-4.12 (m, 1H), 4.11-4.06 (m, 1H), 4.04 (s, 1H), |
| | | | | 4.00 (s, 2H), 3.26 (d, |
| | | | | J = 5.2 Hz, 1H), 3.20 |
| | | | | (d, J = 4.8 Hz, 1H), |
| | | | | 2.33 (s, 3H), 2.21- |
| | | | | 2.13 (m, 1H), 2.12- |
| | | | | 2.06 (m, 1H), 2.05- |
| | | | | 1.97 (m, 2H), 1.97- |
| | | | | 1.91 (m, 2H), 1.90- |
| | | | | 1.73 (m, 4H) |
| 507 | ((2-(((3S,6S,9aS)-3- (3-(6- (dimethylamino)-4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 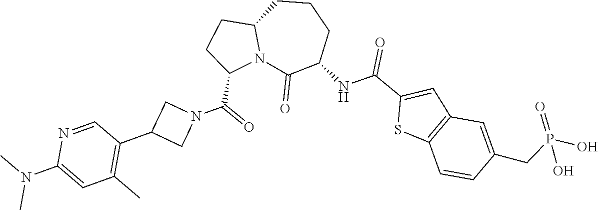 | 640.1 | ¹H NMR (400 MHz, CD₃OD) δ 7.69 (s, 1H), 7.55 (d, J = 8.0 Hz, 1H), 7.50 (s, 1H), 7.32 (d, J = 8.4 Hz, 1H), 7.10 (s, 1H), 6.70-6.62 (m, 1H), 4.68 (d, J = 11.2 Hz, 1H), 4.60 (t, J = 8.0 Hz, 1H), 4.42 (d, J = 6.0 Hz, 1H), 4.29 (t, J = 7.6 Hz, 1H), 4.22- 4.15 (m, 1H), 4.05 (s, 1H), 4.01-3.95 (m, |
| | | | | 1H), 3.18-3.12 (m, |
| | | | | 1H), 3.10 (s, 6H), |
| | | | | 3.03 (d, J = 2.8 Hz, |
| | | | | 1H), 2.34-2.26 (m, |
| | | | | 1H), 2.16-2.05 (m, |
| | | | | 3H), 2.05-1.95 (m, |
| | | | | 3H), 1.89 (s, 3H), |
| | | | | 1.83 (d, J = 3.6 Hz, |
| | | | | 3H) |
| 508 | ((2-(((3S,6S,9aS)-5- oxo-3-(3-(pyridin-3- yl)pyrrolidine-1- carbonyl)octahydro- 1H-pyrrolo[1,2- a]azepin-6- yl)carbamoyl)benzo [b]thiophen-5- yl)methyl) phosphonic acid | 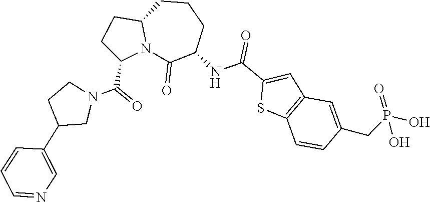 | 597.2 | ¹H NMR (400 MHz, CD₃OD) δ 8.47-8.30 (m, 1H), 8.23-8.09 (m, 1H), 8.04-7.75 (m, 3H), 7.74-7.55 (m, 1H), 7.53-7.32 (m, 2H), 4.85-4.73 (m, 2H), 4.23-3.95 (m, 2H), 3.92-3.57 (m, 2H), 3.55-3.36 (m, 2H), 3.22-3.08 (m, 2H), 2.35-1.85 (m, 12H) |
| 509 | ((2-(((3S,6S,10aS)- 5-oxo-3-(3-(pyridin- 3-yl)pyrrolidine-1- carbonyl)decahydro pyrrolo[1,2- a]azocin-6- yl)carbamoyl)benzo [b]thiophen-5- | 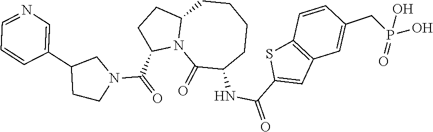 [SMILES: O=C(N[C@H]1CCCC[C@H]2CC[C@@H](C(=O)N3CCC(c4cccnc4)C3)N2C1=O)c1cc2cc(CP(=O)(O)O)ccc2s1] | 611.2 | ¹H NMR (400 MHz, CD₃OD) δ 8.56-8.39 (m, 1H), 8.37-8.12 (m, 1H), 8.08-7.93 (m, 1H), 7.88-7.69 (m, 2H), 7.67-7.50 (m, 1H), 7.49-7.29 (m, 2H), 5.19-5.03 |
| | yl)methyl) | | | (m, 1H), 4.80-4.68 |
| | phosphonic acid | | | (m, 1H), 4.50-4.35 |
| | | | | (m, 1H), 4.18-3.99 |
| | | | | (m, 1H), 3.92-3.77 |
| | | | | (m, 1H), 3.75-3.59 |
| | | | | (m, 1H), 3.56-3.38 |
| | | | | (m, 2H), 3.18-2.99 |
| | | | | (m, 2H), 2.39-1.68 |
| | | | | (m, 14H) |
| 510 | ((2-(((3S,6S,9aS)-3- (3-(5-cyano-4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 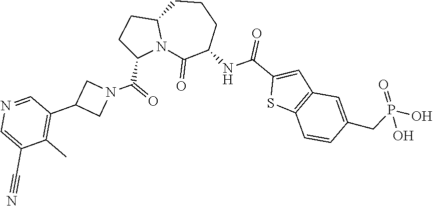 | 622.3 | ¹H NMR (400 MHz, CD₃OD) δ 8.93-8.81 (m, 1H), 8.75 (s, 1H), 8.00 (m, 1H), 7.88 (m, 1H), 7.78 (m, 1H), 7.49 (m, 1H), 4.73 (s, 1H), 4.63- 4.52 (m, 2H), 4.48- 4.42 (m, 1H), 4.33- 4.26 (m, 1H), 4.23- 4.06 (m, 2H), 3.72 (m, 1H), 3.22 (m, 1H), 3.14-3.08 (m, |
| | | | | 1H), 2.51 (d, J = 2.4 |
| | | | | Hz, 3H), 2.36-2.29 |
| | | | | (m, 1H), 2.14-2.04 |
| | | | | (m, 2H), 2.03-1.96 |
| | | | | (m, 2H), 1.92-1.85 |
| | | | | (m, 2H), 1.38 (m, 3H) |
| 511 | ((2-(((3S,6S,9aS)-3- (3-(6-methoxy-4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 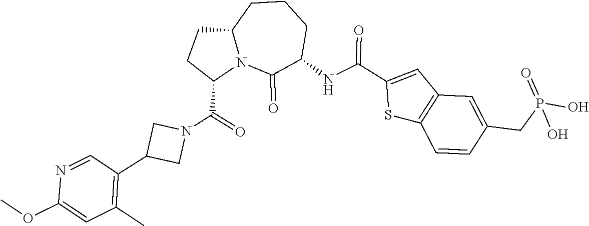 | 627.0 | ¹H NMR (400 MHz, CD₃OD) δ 7.99-8.03 (m, 1H) 7.85 (d, J = 8.00 Hz, 3H) 7.41-7.45 (m, 1H) 6.39-6.47 (m, 1H) 4.75 (d, J=12.00 Hz, 1H) 4.22-4.64 (m, 4H) 3.87-4.14 (m, 3H) 3.46-3.60 (m, 3H) 3.28 (s, 1H) 3.23 (s, 1H) 2.26-2.35 (m, 1H) 2.13-2.16 (m, 3H) 2.13 (s, 2H) |
| | | | | 1.79-2.04 (m, 7H) |
| 512 | ((2-(((3S,6S,9aS)-3- (3-(4- (dimethylamino) pyridin-3- yl)azetidine- 1-carbonyl)-5- oxooctahydro-1H- pyrrolo[1,2- a]azepin-6- yl)carbamoyl)benzo [b]thiophen-5- yl)methyl) phosphonic acid | 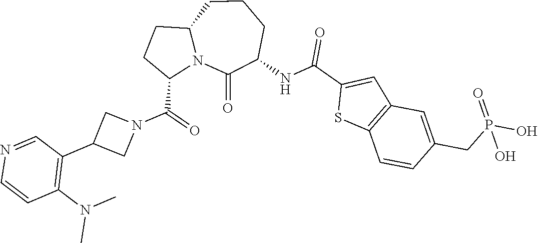 [SMILES: CN(C)c1ccncc1C1CN(C(=O)[C@@H]2CC[C@@H]3CCC[C@H](NC(=O)c4cc5cc(CP(=O)(O)O)ccc5s4)C(=O)N32)C1] | 626.2 | ¹H NMR (400 MHz, CD₃OD) δ 8.33 (d, J = 4.0 Hz, 1H), 8.14- 7.93 (m, 2H), 7.87- 7.78 (m, 2H), 7.46- 7.39 (m, 1H), 7.13- 7.01 (m, 1H), 4.78- 4.66 (m, 2H), 4.60- 4.47 (m, 2H), 4.46- 4.34 (m, 1H), 4.34- 4.25 (m, 1H), 4.15- 4.00 (m, 2H), 3.27- 3.23 (m, 1H), 3.20 (d, J = 4.4 Hz, 1H), 3.15 (d, J = 4.4 Hz, 6H), |
| | | | | 2.39-2.25 (m, 1H), |
| | | | | 2.21-2.06 (m, 2H), |
| | | | | 2.05-1.98 (m, 2H), |
| | | | | 1.93 (dd, J = 6.6, 12.4 |
| | | | | Hz, 2H), 1.89-1.74 |
| | | | | (m, 3H) |
| 513 | (difluoro(2- (((3S,6S,10aS)-3-(3- hydroxy-3-(2-oxo- 1,2-dihydropyridin- 4-yl)pyrrolidine-1- carbonyl)-5- oxodecahydro- pyrrolo[1,2- a]azocin-6- yl)carbamoyl)benzo [b]thiophen-5- yl)methyl) | 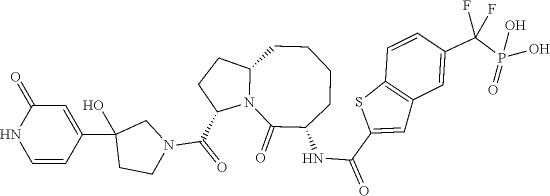 [SMILES: O=C(N[C@H]1CCCC[C@H]2CC[C@@H](C(=O)N3CCC(O)(c4cc[nH]c(=O)c4)C3)N2C1=O)c1cc2cc(C(F)(F)P(=O)(O)O)ccc2s1] | 679.3 | ¹H NMR (400 MHz, CD₃OD) δ 8.15 (s, 2H), 8.04-7.98 (m, 1H), 7.71-7.65 (m, 1H), 7.53-7.38 (m, 1H), 6.89-6.74 (m, 1H), 6.64 (t, J = 5.6 Hz, 1H), 5.08-4.98 (m, 1H), 4.46-4.40 (m, 1H), 4.21-3.99 (m, 1H), 3.95 (s, 2H), 3.69-3.46 (m, 2H), |
| | phosphonic acid | | | 2.34-2.23 (m, 2H), |
| | | | | 2.14-1.93 (m, 8H), |
| | | | | 1.89-1.78 (m, 2H), |
| | | | | 1.73-1.62 (m, 2H) |
| 514 | ((2-(((3S,6S,9aS)-3- (3-(4-fluoropyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 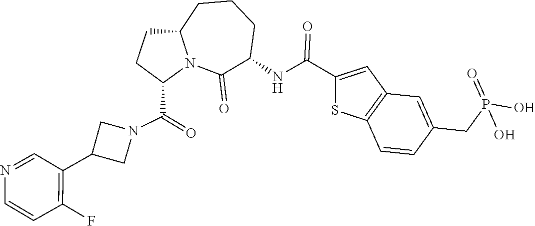 [SMILES: O=C(N[C@H]1CCC[C@H]2CC[C@@H](C(=O)N3CC(c4cnccc4F)C3)N2C1=O)c1cc2cc(CP(=O)(O)O)ccc2s1] | 601.0 | ¹H NMR (400 MHz, CD₃OD) 8.90-8.71 (m, 1H), 8.65-8.58 (m, 1H), 7.99 (d, J = 12.4Hz, 1H), 7.87- 7.75 (m, 2H), 7.55- 7.47 (m, 1H), 7.44- 7.38 (m, 1H), 4.97- 4.90 (m, 1H), 4.76- 4.67 (m, 2H), 4.56 (m, 1H), 4.48-4.39 (m, 1H), 4.30-4.12 (m, 2H), 4.07 (d, J = 5.2Hz, 1H), 3.27 (d, |
| | | | | J = 2.4Hz, 1H), 3.22 (d, |
| | | | | J = 2.4Hz, 1H), 2.36- |
| | | | | 2.25 (m, 1H), 2.19- |
| | | | | 2.11 (m, 1H), 2.10- |
| | | | | 2.00 (m, 2H), 1.96 (s, |
| | | | | 2H), 1.92-1.85 (m, |
| | | | | 2H), 1.83 (s, 2H) |
| 515 | ((2-(((3S,6S,9aS)-3- (3-(2- cyanopyrimidin-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 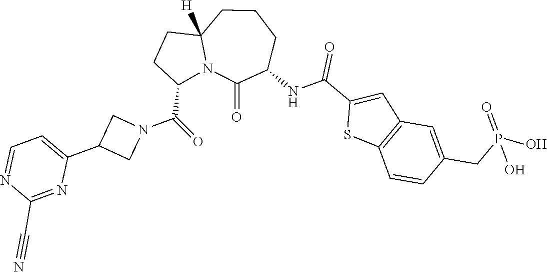 [SMILES: N#Cc1nccc(C2CN(C(=O)[C@@H]3CC[C@@H]4CCC[C@H](NC(=O)c5cc6cc(CP(=O)(O)O)ccc6s5)C(=O)N43)C2)n1] | 608.9 | ¹H NMR (400 MHz, DMSO-d₆) δ 8.94 (dd, J = 5.2, 10.0 Hz, 1H), 8.69 (t, J = 6.4 Hz, 1H), 8.16 (d, J = 11.6 Hz, 1H), 7.97- 7.84 (m, 2H), 7.78 (s, 1H), 7.36 (d, J = 8.4 Hz, 1H), 4.82-4.33 (m, 5H), 4.31-3.93 (m, 5H), 3.17-2.91 (m, 2H), 2.26-2.15 (m, 1H), 2.11-1.91 (m, 2H), 1.90-1.62 (m, 7H) |
| 516 | (difluoro(2- (((3S,6S,10aS)-5- oxo-3-(3-(4- phenylpyridin-3- yl)azetidine-1- carbonyl)decahydro pyrrolo[1,2- a]azocin-6- yl)carbamoyl)benzo [b]thiophen-5- yl)methyl) phosphonic acid | 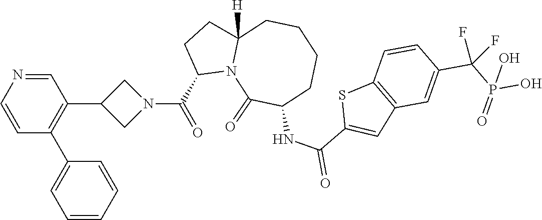 [SMILES: O=C(N[C@H]1CCCC[C@H]2CC[C@@H](C(=O)N3CC(c4cnccc4-c4ccccc4)C3)N2C1=O)c1cc2cc(C(F)(F)P(=O)(O)O)ccc2s1] | 709.2 | ¹H NMR (400 MHz, CD₃OD) δ 8.80-8.67 (m, 1H), 8.37-8.15 (m, 1H), 8.08-7.95 (m, 1H), 7.92 (d, J = 8.8 Hz, 1H), 7.89- 7.75 (m, 1H), 7.74- 7.58 (m, 2H), 7.58- 7.52 (m, 2H), 7.47- 7.42 (m, 1H), 7.35- 7.30 (m, 1H), 7.26- 7.16 (m, 1H), 4.98- 4.93 (m, 1H), 4.53- |
| | | | | 4.45 (m, 1H), 4.44- |
| | | | | 4.31 (m, 3H), 4.11- |
| | | | | 3.94 (m, 2H), 3.94- |
| | | | | 3.64 (m, 1H), 2.30- |
| | | | | 2.13 (m, 2H), 2.07- |
| | | | | 1.89 (m, 4H), 1.88- |
| | | | | 1.73 (m, 4H), 1.71- |
| | | | | 1.65 (m, 1H), 1.58 (d, |
| | | | | J = 2.4 Hz, 1H) |
| 517 | ((2-(((3S,6S,9aS)-3- (3-(4- cyclopropylpyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 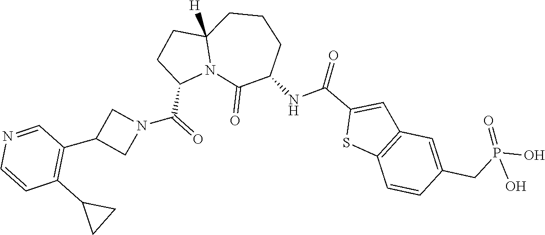 [SMILES: O=C(N[C@H]1CCC[C@H]2CC[C@@H](C(=O)N3CC(c4cnccc4C4CC4)C3)N2C1=O)c1cc2cc(CP(=O)(O)O)ccc2s1] | 623.1 | ¹H NMR (400 MHz, CD₃OD) δ 8.72-8.61 (m, 1H), 8.55-8.38 (m, 1H), 8.03-7.92 (m, 1H), 7.89-7.76 (m, 2H), 7.50-7.32 (m, 2H), 4.86-4.66 (m, 3H), 4.61-4.46 (m, 3H), 4.28-4.18 (m, 1H), 4.09 (s, 1H), 3.28-3.16 (m, 2H), 2.38-2.26 (m, 1H), 2.21-1.95 (m, 6H), 1.95-1.80 (m, 4H), |
| | | | | 1.50-1.37 (m, 2H), |
| | | | | 1.19-1.05 (m, 2H) |
| 518 | (difluoro(2- (((3S,6S,10aS)-5- oxo-3-(3-(5- phenylpyridin-3- yl)azetidine-1- carbonyl)decahydro pyrrolo[1,2- a]azocin-6- yl)carbamoyl)benzo [b]thiophen-5- yl)methyl) phosphonic acid | 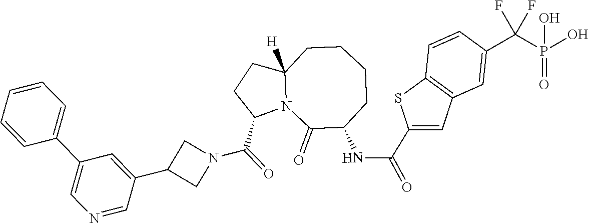 [SMILES: O=C(N[C@H]1CCCC[C@H]2CC[C@@H](C(=O)N3CC(c4cncc(-c5ccccc5)c4)C3)N2C1=O)c1cc2cc(C(F)(F)P(=O)(O)O)ccc2s1] | 709.3 | ¹H NMR (400 MHz, CD₃OD) δ 9.01-8.92 (m, 1H), 8.91-8.82 (m, 1H), 8.79-8.67 (m, 1H), 8.15-8.03 (m, 1H), 7.96-7.86 (m, 1H), 7.84-7.74 (m, 3H), 7.72-7.65 (m, 1H), 7.63-7.54 (m, 1H), 7.50-7.36 (m, 2H), 5.09-4.92 (m, 2H), 4.79-4.73 (m, 1H), 4.51-4.37 (m, 3H), 4.24-4.11 |
| | | | | (m, 2H), 2.31-2.18 |
| | | | | (m, 2H), 2.13-1.96 |
| | | | | (m, 5H), 1.94-1.77 |
| | | | | (m, 3H), 1.75-1.60 |
| | | | | (m, 2H) |
| 519 | ((2-(((3S,6S,9aS)-3- (3-(4-chloropyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 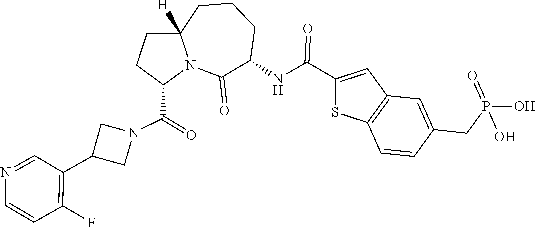 [SMILES: O=C(N[C@H]1CCC[C@H]2CC[C@@H](C(=O)N3CC(c4cnccc4F)C3)N2C1=O)c1cc2cc(CP(=O)(O)O)ccc2s1] | 616.9 | ¹H NMR (400 MHz, CD₃OD) δ 8.83-8.70 (m, 1H), 8.51-8.46 (m, 1H), 8.05-7.95 (m, 1H), 7.89-7.78 (m, 2H), 7.71-7.69 (m, 1H), 7.47-7.38 (m, 1H), 5.02-4.90 (m, 1H), 4.78-4.68 (m, 2H), 4.61-4.53 (m, 1H), 4.52-4.42 (m, 1H), 4.34 (t, J = 8.0 Hz, 1H), 4.24- 4.17 (m, 1H), 4.07 (d, |
| | | | | J = 4.0 Hz, 1H), 3.28- |
| | | | | 3.19 (m, 2H), 2.36- |
| | | | | 2.25 (m, 1H), 2.21- |
| | | | | 2.05 (m, 2H), 2.04- |
| | | | | 1.93 (m, 3H), 1.92- |
| | | | | 1.73 (m, 4H) |
| 520 | ((2-(((3S,6S,9aS)-3- (3-(4-cyanopyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 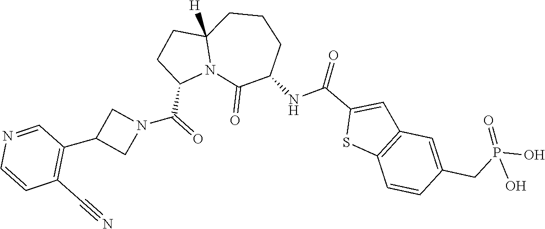 [SMILES: N#Cc1ccncc1C1CN(C(=O)[C@@H]2CC[C@@H]3CCC[C@H](NC(=O)c4cc5cc(CP(=O)(O)O)ccc5s4)C(=O)N32)C1] | 608.0 | ¹H NMR (400 MHz, CD₃OD) δ 8.89-9.15 (m, 1H) 8.69 (m, 1H) 8.02 (d, J = 4.00 Hz, 1H) 7.86 (m, 2H) 7.74 (m, 1H) 7.38- 7.49 (m, 1H) 5.00 (s, 1H) 4.70-4.83 (m, 2H) 4.51-4.63 (m, 2H) 4.30-4.41 (m, 1H) 4.19-4.27 (m, 1H) 4.03-4.12 (m, 1H) 3.28 (s, 1H) 3.23 (s, 1H) 2.25-2.38 (m, |
| | | | | 1H) 2.11-2.17 (m, |
| | | | | 1H) 1.98-2.08 (m, |
| | | | | 3H) 1.75-1.96 (m, |
| | | | | 5H) |
| 521 | ((2-(((3S,6S,10aS)- 3-(((3H- imidazo[4,5- b]pyridin-6- yl)methyl) carbamoyl)-5- oxodecahydro- pyrrolo[1,2- a]azocin-6- yl)carbamoyl)benzo [b]thiophen-5- yl)fluoromethyl) | 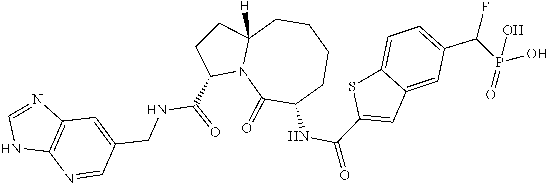 [SMILES: O=C(N[C@H]1CCCC[C@H]2CC[C@@H](C(=O)NCc3cnc4[nH]cnc4c3)N2C1=O)c1cc2cc(C(F)P(=O)(O)O)ccc2s1] | 629.1 | ¹H NMR (400 MHz, DMSO-d6) 8 8.88 (d, J = 7.2 Hz, 1H), 8.59 (t, J = 5.6 Hz, 1H), 8.45 (s, 1H), 8.37- 8.21 (m, 2H), 8.13- 8.03 (m, 1H), 8.03- 7.89 (m, 2H), 7.61- 7.50 (m, 1H), 5.91- 5.73 (m, 1H), 4.99- 4.90 (m, 1H), 4.55- 4.49 (m, 1H), 4.40- |
| | phosphonic acid | | | 4.32 (m, 2H), 4.31- |
| | | | | 4.26 (m, 1H), 3.90 (s, |
| | | | | 1H), 2.20-2.10 (m, |
| | | | | 1H), 2.09-2.02 (m, |
| | | | | 1H), 2.02-1.95 (m, |
| | | | | 1H), 1.94-1.76 (m, |
| | | | | 5H), 1.74-1.69 (m, |
| | | | | 1H), 1.68-1.43 (m, |
| | | | | 3H) |
| 522 | (fluoro(2- (((3S,6S,10aS)-3- (((R)-1- (methoxycarbonyl) pyrrolidin-3- yl)carbamoyl)-5- oxodecahydro- pyrrolo[1,2- a]azocin-6- yl)carbamoyl)benzo [b]thiophen-5- yl)methyl) phosphonic acid | 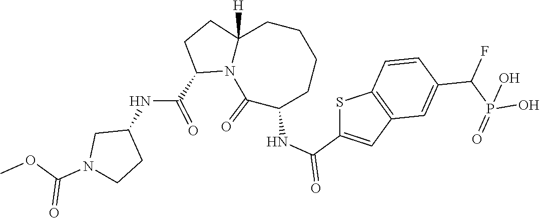 [SMILES: COC(=O)N1CC[C@@H](NC(=O)[C@@H]2CC[C@@H]3CCCC[C@H](NC(=O)c4cc5cc(C(F)P(=O)(O)O)ccc5s4)C(=O)N32)C1] | 625.1 | ¹H NMR (400 MHz, CD₃OD) δ 8.08 (s, 1H), 7.99 (s, 1H), 7.92 (d, J = 8.4 Hz, 1H), 7.57 (d, J = 8.4 Hz, 1H), 5.91-5.75 (m, 1H), 5.06-5.00 (m, 1H), 4.43-4.35 (m, 2H), 4.34-4.28 (m, 1H), 3.65 (d, J = 2.0 Hz, 3H), 3.63- 3.59 (m, 1H), 3.51- 3.40 (m, 2H), 3.27 (d, |
| | | | | J = 4.0 Hz, 1H), 2.25- |
| | | | | 2.17 (m, 2H), 2.16- |
| | | | | 2.09 (m, 1H), 2.08- |
| | | | | 2.03 (m, 1H), 2.02- |
| | | | | 1.92 (m, 5H), 1.90- |
| | | | | 1.78 (m, 3H), 1.72- |
| | | | | 1.59 (m, 2H) |
| 523 | (fluoro(2- (((3S,6S,10aS)-3-(1- (methoxycarbonyl) azetidin-3- yl)carbamoyl)-5- oxodecahydro- pyrrolo[1,2- a]azocin-6- yl)carbamoyl)benzo [b]thiophen-5- yl)methyl) phosphonic acid | 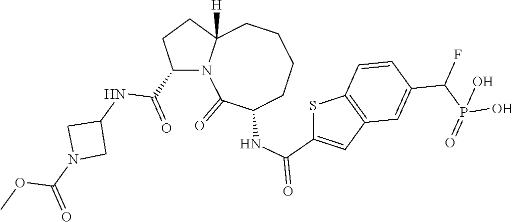 [SMILES: COC(=O)N1CC(NC(=O)[C@@H]2CC[C@@H]3CCCC[C@H](NC(=O)c4cc5cc(C(F)P(=O)(O)O)ccc5s4)C(=O)N32)C1] | 611.0 | ¹H NMR (400 MHz, CD₃OD) δ 8.10 (s, 1H), 8.02 (s, 1H), 7.95 (d, J = 8.8 Hz, 1H), 7.58 (m, 1H), 5.94-5.74 (m, 1H), 5.02 (s, 1H), 4.57- 4.49 (m, 1H), 4.37 (m, 2H), 4.26 (m, 2H), 3.92-3.82 (m, 2H), 3.65 (s, 3H), 2.24 (s, 2H), 2.04- 1.94 (m, 6H), 1.84- 1.76 (m, 2H), 1.72- |
| | | | | 1.62 (m, 2H) |
| 524 | ((2-(((3S,6S,10aS)- 3-((1- ((benzyloxy) carbonyl)azetidin- 3-yl)carbamoyl)-5- oxodecahydro- pyrrolo[1,2- a]azocin-6- yl)carbamoyl)benzo [b]thiophen-5- yl)fluoromethyl) phosphonic acid | 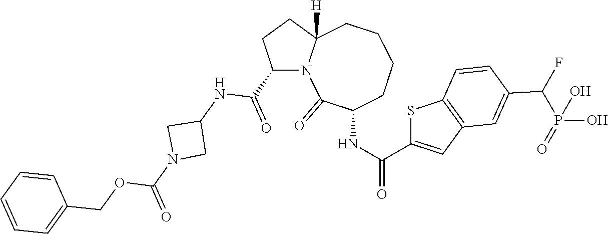 [SMILES: O=C(N[C@H]1CCCC[C@H]2CC[C@@H](C(=O)NC3CN(C(=O)OCc4ccccc4)C3)N2C1=O)c1cc2cc(C(F)P(=O)(O)O)ccc2s1] | 687.2 | ¹H NMR (400 MHz, CD₃OD) δ 8.14-8.05 (m, 1H), 8.02 (s, 1H), 7.97-7.91 (m, 1H), 7.62-7.56 (m, 1H), 7.38-7.26 (m, 5H), 5.92-5.69 (m, 1H), 5.08 (s, 2H), 5.05- 4.99 (m, 1H), 4.60- 4.48 (m, 1H), 4.43- 4.33 (m, 2H), 4.32- 4.24 (m, 2H), 3.98- 3.83 (m, 2H), 2.27- 2.17 (m, 2H), 2.08- |
| | | | | 1.88 (m, 7H), 1.86- |
| | | | | 1.75 (m, 2H), 1.71- |
| | | | | 1.64 (m, 1H) |
| 525 | (fluoro(2- (((3S,6S,10aS)-3- (((3S,4S)-4-fluoro-1- (methoxycarbonyl) pyrrolidin-3- yl)carbamoyl)-5- oxodecahydro- pyrrolo[1,2-a]azocin- 6-yl)carbamoyl) benzo[b]thiophen-5- yl)methyl) phosphonic acid | 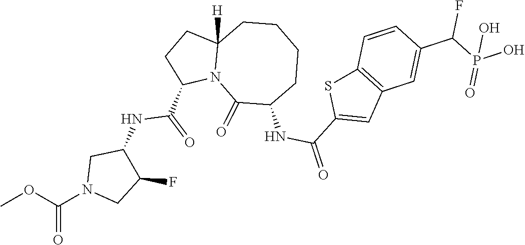 [SMILES: COC(=O)N1C[C@H](NC(=O)[C@@H]2CC[C@@H]3CCCC[C@H](NC(=O)c4cc5cc(C(F)P(=O)(O)O)ccc5s4)C(=O)N32)[C@@H](F)C1] | 643.0 | ¹H NMR (400 MHz, CD₃OD) δ 8.10 (s, 1H), 8.01 (s, 1H), 7.94 (d, J = 8.00 Hz, 1H), 7.58 (d, J = 8.00 Hz, 1H), 5.74-5.92 (m, 1H), 5.00-5.05 (m, 1H), 4.28-4.54 (m, 4H), 3.68-3.72 (m, 5H), 3.58-3.61 (m, 1H), 3.50-3.56 (m, 1H), 2.17-2.24 (m, 2H), 2.00 (m, 6H), 1.82 (m, 2H), 1.61-1.73 (m, 2H) |
| 526 | ((2-(((3S,6S,9aS)-3- (3-(6-cyanopyridin- 2-yl)azetidine-1- carbonyl)-5- oxooctahydro-1H- pyrrolo[1,2- a[azepin-6- yl)carbamoyl)benzo [b]thiophen-5- yl)fluoromethyl) phosphonic acid | 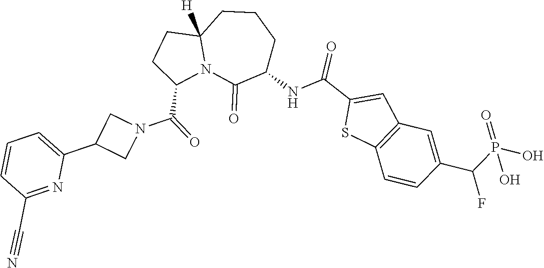 [SMILES: N#Cc1cccc(C2CN(C(=O)[C@@H]3CC[C@@H]4CCC[C@H](NC(=O)c5cc6cc(C(F)P(=O)(O)O)ccc6s5)C(=O)N43)C2)n1] | 626.1 | 1H NMR (400 MHz, CD₃OD) δ 8.08-8.01 (m, 2H), 7.97-7.85 (m, 2H), 7.79-7.73 (m, 1H), 7.64-7.60 (m, 2H), 5.76-5.60 (m, 1H), 4.77-4.64 (m, 2H), 4.61-4.58 (m, 1H), 4.50-4.26 (m, 2H), 4.19-3.99 (m, 3H), 2.37-2.26 (m, 1H), 2.14 (d, J = 7.6 Hz, 1H), 2.04- 1.99 (m, 3H), 1.90 (s, 3H), 1.84 (s, 2H) |
| 527 | ((2-(((3S,6S,10aS)- 3-(((R)-1- (cyclopropoxy- carbonyl)pyrrolidin- 3-yl)carbamoyl)-5- oxodecahydro- pyrrolo[1,2- a]azocin-6- yl)carbamoyl)benzo [b]thiophen-5- yl)fluoromethyl) phosphonic acid | 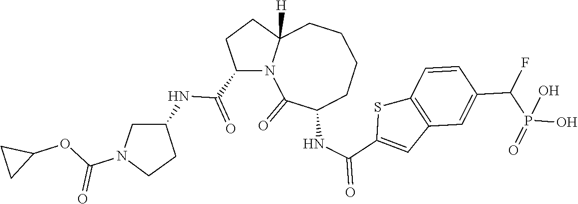 [SMILES: O=C(N[C@H]1CCCC[C@H]2CC[C@@H](C(=O)N[C@@H]3CCN(C(=O)OC4CC4)C3)N2C1=O)c1cc2cc(C(F)P(=O)(O)O)ccc2s1] | 651.1 | ¹H NMR (400 MHz, CD₃OD) δ 8.09 (s, 1H), 8.01 (s, 1H), 7.93 (d, J = 8.4 Hz, 1H), 7.58 (d, J = 8.4 Hz, 1H), 5.90-5.75 (m, 1H), 5.08-4.98 (m, 1H), 4.44-4.28 (m, 3H), 4.02-3.94 (m, 1H), 3.66-3.52 (m, 1H), 3.51-3.43 (m, 1H), 3.42-3.36 |
| | | | | (m, 1H), 3.29-3.17 |
| | | | | (m, 1H), 2.27-2.17 |
| | | | | (m, 2H), 2.12 (dd, J = |
| | | | | 6.0, 12.8 Hz, 1H), |
| | | | | 2.08-1.92 (m, 6H), |
| | | | | 1.92-1.85 (m, 1H), |
| | | | | 1.81 (dd, J = 6.8, 10.4 |
| | | | | Hz, 2H), 1.72-1.56 |
| | | | | (m, 2H), 0.65 (d, J = |
| | | | | 3.2 Hz, 4H) |
| 528 | ((2-(((3S,6S,10aS)- 3-((1- (cyclopropoxy- carbonyl)azetidin- 3-yl)carbamoyl)-5- oxodecahydro- pyrrolo[1,2- a]azocin-6- yl)carbamoyl)benzo [b]thiophen-5- yl)fluoromethyl) phosphonic acid | 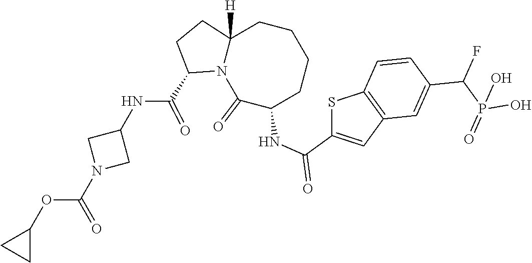 [SMILES: O=C(N[C@H]1CCCC[C@H]2CC[C@@H](C(=O)NC3CN(C(=O)OC4CC4)C3)N2C1=O)c1cc2cc(C(F)P(=O)(O)O)ccc2s1] | 637.2 | ¹H NMR (400 MHz, DMSO-d₆) δ 8.82 (d, J = 6.8 Hz, 1H), 8.69 (d, J = 6.8 Hz, 1H), 8.25 (s, 1H), 8.08- 7.94 (m, 2H), 7.52 (d, J = 8.4 Hz, 1H), 5.91- 5.74 (m, 1H), 4.95- 4.86 (m, 1H), 4.42- 4.32 (m, 1H), 4.29- 4.18 (m, 2H), 4.10 (t, J = 8.0 Hz, 2H), 3.93 (s, 1H), 3.69 (dt, J = 5.2, 9.2 Hz, 2H), 2.14- 1.98 (m, 2H), 1.92- 1.74 (m, 6H), 1.73- |
| | | | | 1.61 (m, 2H), 1.60- |
| | | | | 1.44 (m, 2H), 0.65- |
| | | | | 0.52 (m, 4H) |
| 529 | ((2-(((3S,6S,10aS)- 3-(((R)-1- (dimethylcarbamoyl) pyrrolidin-3- yl)carbamoyl)-5- oxodecahydro- pyrrolo[1,2- a]azocin-6- yl)carbamoyl)benzo [b]thiophen-5- yl)fluoromethyl) phosphonic acid | 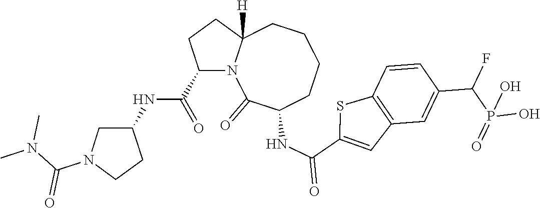 [SMILES: CN(C)C(=O)N1CC[C@@H](NC(=O)[C@@H]2CC[C@@H]3CCCC[C@H](NC(=O)c4cc5cc(C(F)P(=O)(O)O)ccc5s4)C(=O)N32)C1] | 638.0 | ¹H NMR (400 MHz, CD₃OD) δ 8.11-8.04 (m, 1H), 8.00 (s, 1H), 7.92 (d, J = 8.4 Hz, 1H), 7.58 (d, J = 8.8 Hz, 1H), 5.69 (s, 1H), 5.07-4.98 (m, 1H), 4.48-4.34 (m, 2H), 4.34-4.24 (m, 1H), 3.72-3.62 (m, 1H), 3.55-3.38 (m, 2H), 3.27-3.21 (m, 1H), 2.83 (s, 6H), 2.28- |
| | | | | 1.90 (m, 7H), 1.88- |
| | | | | 1.75 (m, 3H), 1.73- |
| | | | | 1.55 (m, 2H) |
| 530 | (fluoro(2- (((3S,6S,10aS)-3- (((R)-1- (methylcarbamoyl) pyrrolidin-3- yl)carbamoyl)-5- oxodecahydro- pyrrolo[1,2- a]azocin-6- yl)carbamoyl)benzo [b]thiophen-5- yl)methyl) phosphonic acid | 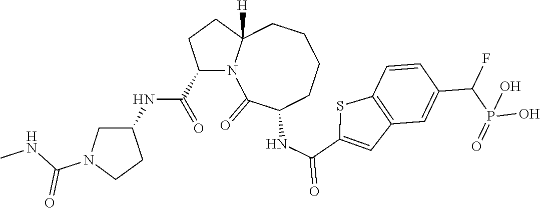 [SMILES: CNC(=O)N1CC[C@@H](NC(=O)[C@@H]2CC[C@@H]3CCCC[C@H](NC(=O)c4cc5cc(C(F)P(=O)(O)O)ccc5s4)C(=O)N32)C1] | 623.9 | ¹H NMR (400 MHz, CD₃OD) δ 8.08 (s, 1H), 8.05-7.97 (m, 1H), 7.92 (d, J = 8.4 Hz, 1H), 7.64-7.55 (m, 1H), 5.88-5.68 (m, 1H), 5.09-4.95 (m, 1H), 4.46-4.29 (m, 3H), 3.64-3.53 (m, 1H), 3.50-3.35 (m, 3H), 3.24-3.21 (m, 1H), 2.70 (s, 3H), 2.25-2.09 (m, 3H), |
| | | | | 2.09-1.86 (m, 7H), |
| | | | | 1.86-1.73 (m, 2H), |
| | | | | 1.73-1.55 (m, 2H) |
| 531 | (fluoro(2- (((3S,6S,9aS)-3- (((1R,3S)-3- hydroxycyclopentyl) carbamoyl)-5- oxooctahydro-1H- pyrrolo[1,2- a]azepin-6- yl)carbamoyl)benzo [b]thiophen-5- yl)methyl) phosphonic acid | 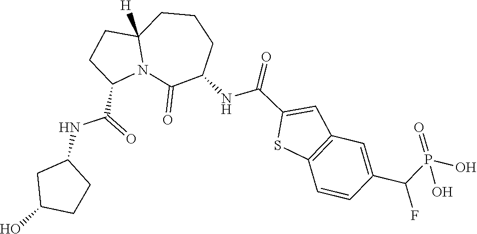 [SMILES: O=C(N[C@H]1CCC[C@H]2CC[C@@H](C(=O)N[C@@H]3CC[C@H](O)C3)N2C1=O)c1cc2cc(C(F)P(=O)(O)O)ccc2s1] | 568.1 | ¹H NMR (400 MHz, CD₃OD) δ 8.07 (s, 1H), 8.03 (s, 1H), 7.97-7.93 (m, 1H), 7.61-7.57 (m, 1H), 5.87 (d, J = 8.0 Hz, 1H), 4.76-4.70 (m, 1H), 4.55-4.50 (m, 1H), 4.31-4.26 (m, 1H), 4.23-4.16 (m, 1H), 4.05-3.98 (m, 1H), 2.31-2.23 (m, 1H), 2.16-1.98 (m, 7H), 1.89 (d, J = 10.4 |
| | | | | Hz, 2H), 1.84-1.78 |
| | | | | (m, 2H), 1.76-1.65 |
| | | | | (m, 3H), 1.61-1.53 |
| | | | | (m, 1H) |
| 532 | ((2-(((3S,6S,10aS)- 3-(((R)-1- carbamoylpyrrolidin- 3-yl)carbamoyl)-5- oxodecahydro-pyrrolo [1,2-a]azocin-6- yl)carbamoyl)benzo [b]thiophen-5- yl)fluoromethyl) phosphonic acid | 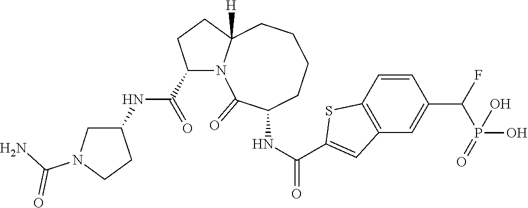 [SMILES: NC(=O)N1CC[C@@H](NC(=O)[C@@H]2CC[C@@H]3CCCC[C@H](NC(=O)c4cc5cc(C(F)P(=O)(O)O)ccc5s4)C(=O)N32)C1] | 610.0 | ¹H NMR (400 MHz, CD₃OD) δ 8.11 (s, 1H), 8.02 (s, 1H), 7.95 (d, J = 8.4 Hz, 1H), 7.59 (d, J = 8.4 Hz, 1H), 6.02-5.71 (m, 1H), 5.10-5.02 (m, 1H), 4.51-4.25 (m, 3H), 3.63 (dd, J = 6.4, 10.8 Hz, 1H), 3.53-3.42 (m, 2H), 3.27 (dd, J = 3.6, 10.4 Hz, 1H), 2.33-2.14 |
| | | | | (m, 3H), 2.09-1.76 |
| | | | | (m, 9H), 1.74-1.55 |
| | | | | (m, 2H) |
| 533 | (fluoro(2- (((3S,6S,10aS)-3- (((R)-1-(1-methyl- 1H-pyrazol-3- yl)pyrrolidin-3- yl)carbamoyl)-5- oxodecahydro- pyrrolo[1,2- a]azocin-6- yl)carbamoyl)benzo [b]thiophen-5- yl)methyl) phosphonic acid | 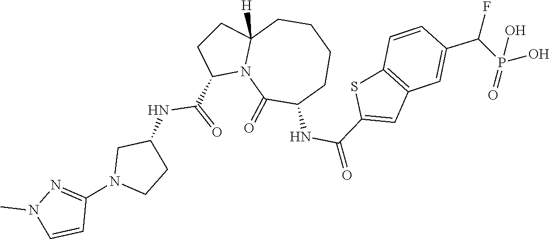 [SMILES: Cn1ccc(N2CC[C@@H](NC(=O)[C@@H]3CC[C@@H]4CCCC[C@H](NC(=O)c5cc6cc(C(F)P(=O)(O)O)ccc6s5)C(=O)N43)C2)n1] | 647.5 | ¹H NMR (400 MHz, CD₃OD) δ 8.06 (s, 1H), 8.01 (d, J = 6.2 Hz, 2H), 7.85 (d, J = 8.4 Hz, 1H), 7.64- 7.57 (m, 1H), 7.30 (s, 1H), 5.73-5.58 (m, 1H), 5.05-5.00 (m, 1H), 4.45-4.38 (m, 3H), 3.68 (s, 3H), 3.55-3.48 (m, 1H), 3.45-3.39 (m, 1H), 3.22-3.18 (m, 1H), 3. 17-3.15 (m, 1H), 2.30-2.15 (m, 3H), |
| | | | | 2.10-2.04 (m, 1H), |
| | | | | 1.99-1.91 (m, 6H), |
| | | | | 1.84-1.77 (m, 2H), |
| | | | | 1.68 (d, J = 15.6 Hz, |
| | | | | 2H) |
| 534 | (fluoro(2- (((3S,6S,10aS)-3- (((R)-1-(3- methylpyridin-2- yl)pyrrolidin-3- yl)carbamoyl)-5- oxodecahydro- pyrrolo [1,2-a]azocin-6- yl)carbamoyl)benzo [b]thiophen-5- yl)methyl) phosphonic acid | 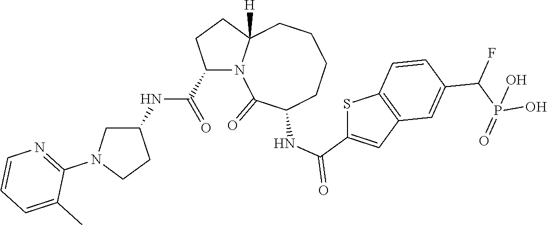 [SMILES: Cc1cccnc1N1CC[C@@H](NC(=O)[C@@H]2CC[C@@H]3CCCC[C@H](NC(=O)c4cc5cc(C(F)P(=O)(O)O)ccc5s4)C(=O)N32)C1] | 658.0 | ¹H NMR (400 MHz, CD₃OD) δ 8.02-7.9 (m, 2H), 7.7 (d, J = 8.0 Hz, 1H), 7.7-7.5 (m, 2H), 7.4 (s, 1H), 6.6-6.4 (m, 1H), 5.7- 5.5 (m, 1H), 5.0- 4.9 (m, 1H), 4.5 (d, J = 4.0 Hz, 1H), 4.5- 4.4 (m, 2H), 4.0 (td, J = 4.0, 10.6 Hz, 1H), 4.0-3.9 (m, 1H), 3.7- 3.6 (m, 2H), 2.4 (d, J = 4.0 Hz, 3H), 2.3- |
| | | | | 2.2 (m, 3H), 2.1- |
| | | | | 2.02(m, 3H), 2.0-1.9 |
| | | | | (m, 4H), 1.9-1.8 (m, |
| | | | | 2H), 1.8-1.6 (m, 2H) |
| 535 | (fluoro(2- (((3S,6S,9aS)-3- (((1R,3S)-3- methoxycyclopentyl) carbamoyl)-5- oxooctahydro-1H- pyrrolo[1,2- a]azepin-6- yl)carbamoyl)benzo [b]thiophen-5- yl)methyl) phosphonic acid | 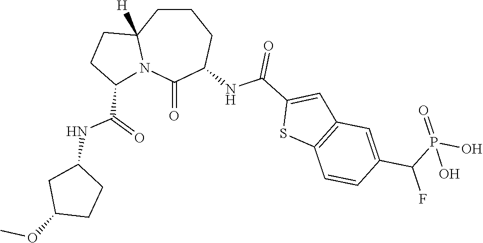 [SMILES: CO[C@H]1CC[C@@H](NC(=O)[C@@H]2CC[C@@H]3CCC[C@H](NC(=O)c4cc5cc(C(F)P(=O)(O)O)ccc5s4)C(=O)N32)C1] | 582.0 | ¹H NMR (400 MHz, CD₃OD) δ 8.09 (s, 1H), 8.06-8.03 (m, 1H), 7.99-7.95 (m, 1H), 7.63-7.58 (m, 1H), 4.77-4.72 (m, 1H), 4.58-4.52 (m, 1H), 4.24-4.16 (m, 1H), 4.08-4.00 (m, 1H), 3.91-3.84 (m, 1H), 2.28 (m, 1H), 2.22-2.15 (m, 1H), 2.11-2.02 (m, 4H), 2.02-1.94 (m, 2H), 1.93-1.87 (m, 2H), |
| | | | | 1.86-1.77 (m, 3H), |
| | | | | 1.77-1.71 (m, 1H), |
| | | | | 1.68-1.59 (m, 2H) |
| 536 | (fluoro(2- (((3S,6S,9aS)-3- (((1R,3R)-3- methoxycyclopentyl) carbamoyl)-5- oxooctahydro-1H- pyrrolo[1,2- a]azepin-6- yl)carbamoyl)benzo [b]thiophen-5- yl)methyl) phosphonic acid | 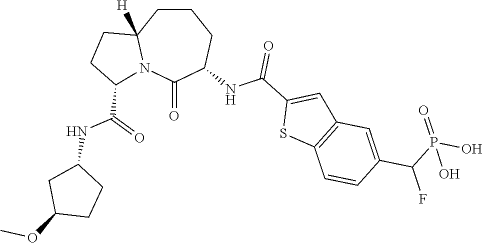 [SMILES: CO[C@@H]1CC[C@@H](NC(=O)[C@@H]2CC[C@@H]3CCC[C@H](NC(=O)c4cc5cc(C(F)P(=O)(O)O)ccc5s4)C(=O)N32)C1] | 582.0 | ¹H NMR (400 MHz, CD₃OD) δ 8.08 (s, 1H), 8.02 (s, 1H), 7.95 (d, J = 8.4 Hz, 1H), 7.59 (d, J = 8.8 Hz, 1H), 5.92-5.73 (m, 1H), 4.74 (d, J = 11.2 Hz, 1H), 4.48 (m, 1H), 4.23 (m, 1H), 4.07-4.00 (m, 1H), 3.91 (m, 1H), 3.26 (s, 3H), 2.27 (m, 1H), 2.10-2.04 (m, 4H), 2.02-1.92 (m, 4H), 1.91-1.81 (m, |
| | | | | 5H), 1.68-1.64 (m, |
| | | | | 1H), 1.55-1.48 (m, |
| | | | | 1H) |
| 537 | (fluoro(2- (((3S,6S,9aS)-3- (((1R,3R)-3- hydroxycyclopentyl )carbamoyl)-5- oxooctahydro-1H- pyrrolo[1,2- a]azepin-6- yl)carbamoyl)benzo [b]thiophen-5- yl)methyl) phosphonic acid | 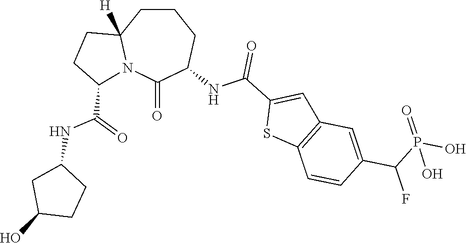 [SMILES: O=C(N[C@H]1CCC[C@H]2CC[C@@H](C(=O)N[C@@H]3CC[C@@H](O)C3)N2C1=O)c1cc2cc(C(F)P(=O)(O)O)ccc2s1] | 568.0 | ¹H NMR (400 MHz, CD₃OD) δ 8.07 (s, 1H), 8.02 (s, 1H), 7.95 (d, J = 8.0 Hz, 1H), 7.59 (d, J = 8.4 Hz, 1H), 5.90-5.73 (m, 1H), 4.74 (d, J = 10.8 Hz, 1H), 4.48 (m, 1H), 4.36-4.29 (m, 1H), 4.36-4.28 (m, 1H), 4.09-3.97 (m, 1H), 2.33-2.21 (m, 1H), 2.20-2.11 (m, 1H), 2.09-1.94 (m, 6H), 1.94-1.80 |
| | | | | (m, 5H), 1.69 (m, |
| | | | | 1H), 1.62-1.46 (m, |
| | | | | 2H) |
| 538 | ((2-(((3S,6S,9aS)-3- (3-(3-fluoropyrazin- 2-yl)azetidine-1- carbonyl)-5- oxooctahydro-1H- pyrrolo[1,2- a]azepin-6- yl)carbamoyl)benzo [b]thiophen-5- yl)methyl) phosphonic acid | 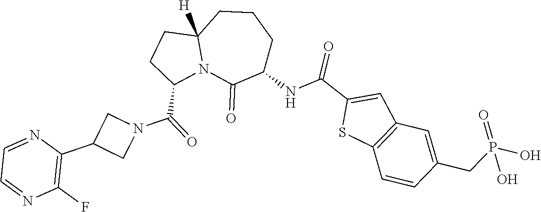 [SMILES: O=C(N[C@H]1CCC[C@H]2CC[C@@H](C(=O)N3CC(c4nccnc4F)C3)N2C1=O)c1cc2cc(CP(=O)(O)O)ccc2s1] | 601.9 | ¹H NMR (400 MHz, CD₃OD) δ 8.59-8.48 (m, 1H), 8.20-8.11 (m, 1H), 8.03-7.94 (m, 1H), 7.88-7.79 (m, 2H), 7.47-7.38 (m, 1H), 4.88 (s, 1H), 4.80-4.63 (m, 2H), 4.61-4.56 (m, 1H), 4.46-4.40 (m, 1H), 4.37-4.22 (m, 2H), 4.06 (s, 1H), 3.29- 3.20 (m, 2H), 2.35- |
| | | | | 2.25 (m, 1H), 2.16- |
| | | | | 2.04 (m, 2H), 2.03- |
| | | | | 1.78 (m, 7H) |
| 539 | ((2-(((3S,6S,9aS)-3- (3-(4- hydroxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 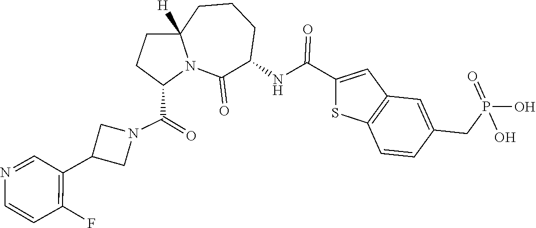 [SMILES: O=C(N[C@H]1CCC[C@H]2CC[C@@H](C(=O)N3CC(c4cnccc4F)C3)N2C1=O)c1cc2cc(CP(=O)(O)O)ccc2s1] | 599.1 | ¹H NMR (400 MHz, CD₃OD) δ 8.33-8.23 (m, 1H), 8.03-7.84 (m, 2H), 7.84-7.63 (m, 2H), 7.45-7.34 (m, 1H), 7.09-6.65 (m, 1H), 4.79-4.65 (m, 2H), 4.60-4.48 (m, 2H), 4.43-4.31 (m, 1H), 4.20-3.99 (m, 3H), 3.28-3.16 (m, 2H), 2.39-2.28 (m, 1H), 2.20-2.06 (m, 2H), 2.06-1.90 |
| | | | | (m, 4H), 1.89-1.73 |
| | | | | (m, 3H) |
| 540 | (fluoro(2- (((3S,6S,9aS)-3-(3- (4-hydroxypyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 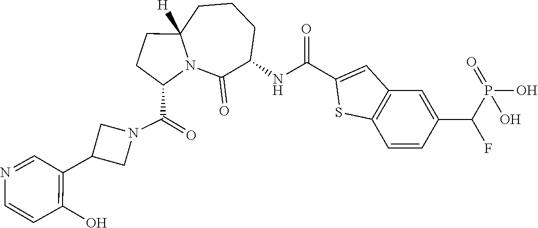 [SMILES: O=C(N[C@H]1CCC[C@H]2CC[C@@H](C(=O)N3CC(c4cnccc4O)C3)N2C1=O)c1cc2cc(C(F)P(=O)(O)O)ccc2s1] | 617.2 | ¹H NMR (400 MHz, CD₃OD) δ 8.34-8.17 (m, 1H), 8.06-7.89 (m, 1H), 7.89 (s, 1H), 7.72-7.57 (m, 2H), 7.10 (d, J = 6.8 Hz, 1H), 6.49 (t, J = 7.6 Hz, 1H), 5.88-5.63 (m, 1H), 4.80-4.68 (m, 2H), 4.61-4.52 (m, 1H), 4.49 (t, J = 7.2 Hz, 1H), 4.41- 4.29 (m, 1H), 4.22- 4.09 (m, 2H), 4.07- |
| | | | | 3.96 (m, 1H), 2.38- |
| | | | | 2.29 (m, 1H), 2.20- |
| | | | | 2.08 (m, 2H), 2.07- |
| | | | | 1.90 (m, 4H), 1.87- |
| | | | | 1.64 (m, 3H) |
| 541 | ((2-(((3S,6S,9aS)-3- (3-(4-fluoropyridin- 2-yl)azetidine-1- carbonyl)-5- oxooctahydro-1H- pyrrolo[1,2- a]azepin-6- yl)carbamoyl)benzo [b]thiophen-5- yl)methyl) phosphonic acid | 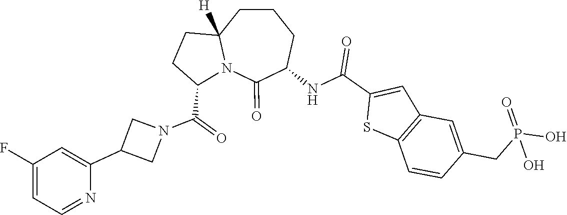 [SMILES: O=C(N[C@H]1CCC[C@H]2CC[C@@H](C(=O)N3CC(c4cc(F)ccn4)C3)N2C1=O)c1cc2cc(CP(=O)(O)O)ccc2s1] | 601.0 | ¹H NMR (400 MHz, DMSO-d6) 8 8.70 (t, J = 6.8 Hz, 1H), 8.62 (m, 1H), 8.17 (d, J = 14.4 Hz, 1H), 7.91- 7.89 (m, 1H), 7.78 (d, J = 5.2 Hz, 1H), 7.40- 7.30 (m, 2H), 7.27- 7.20 (m, 1H), 4.69- 4.60 (m, 1H), 4.58- 4.41 (m, 2H), 4.39- 4.12 (m, 2H), 4.11- 3.94 (m, 3H), 3.11- |
| | | | | 3.03 (m, 2H), 2.26- |
| | | | | 2.13 (m, 1H), 2.11- |
| | | | | 1.93 (m, 2H), 1.92- |
| | | | | 1.76 (m, 7H) |
| 542 | (fluoro(2- (((3S,6S,10aS)-3- (((R)-1-(1-methyl- 1H-pyrazol-5- yl)pyrrolidin-3- yl)carbamoyl)-5- oxodecahydropyrrolo [1,2-a]azocin-6- yl)carbamoyl)benzo [b]thiophen-5- yl)methyl) phosphonic acid | 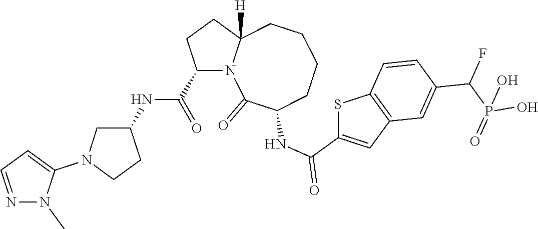 [SMILES: Cn1nccc1N1CC[C@@H](NC(=O)[C@@H]2CC[C@@H]3CCCC[C@H](NC(=O)c4cc5cc(C(F)P(=O)(O)O)ccc5s4)C(=O)N32)C1] | 647.2 | ¹H NMR (400 MHz, DMSO-d₆) δ 8.79 (d, J = 6.8 Hz, 1H), 8.27 (d, J = 7.2 Hz, 1H), 8.25 (s, 1H), 8.03 (d, J = 8.4 Hz, 1H), 7.96 (s, 1H), 7.52 (d, J = 8.8 Hz, 1H), 7.24 (d, J = 2.0 Hz, 1H), 5.93- 5.74 (m, 1H), 5.67 (d, J = 2.0 Hz, 1H), 4.97-4.86 (m, 1H), 4.35-4.24 (m, 3H), 3.66 (s, 3H), 3.31 (m, |
| | | | | 1H), 3.09 (m, 1H), |
| | | | | 2.97 (m, 1H), 2.23- |
| | | | | 2.16 (m, 1H), 2.14- |
| | | | | 2.01 (m, 2H), 1.78 (s, |
| | | | | 7H), 1.71 (s, 4H) |
| 543 | ((2-(((3S,6S,9aS)-3- (3-(4- hydroxypyridin-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 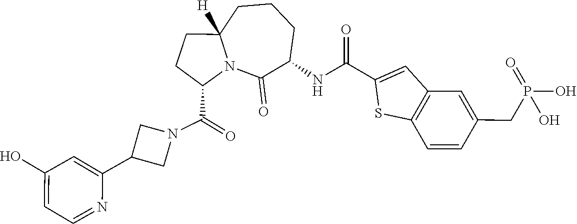 [SMILES: O=C(N[C@H]1CCC[C@H]2CC[C@@H](C(=O)N3CC(c4cc(O)ccn4)C3)N2C1=O)c1cc2cc(CP(=O)(O)O)ccc2s1] | 599.2 | ¹H NMR (400 MHz, CD₃OD) δ 8.04-7.99 (m, 1H), 7.91-7.84 (m, 1H), 7.82-7.67 (m, 2H), 7.48-7.42 (m, 1H), 6.71-6.55 (m, 1H), 6.48-6.42 (m, 1H), 4.79-4.70 (m, 1H), 4.60-4.50 (m, 1H), 4.41 (s, 2H), 4.32-4.23 (m, 1H), 4.10-4.05 (m, 1H), 4.04-3.96 (m, 1H), 3.86-3.76 (m, 1H), |
| | | | | 3.19-3.07 (m, 2H), |
| | | | | 2.36-2.25 (m, 1H), |
| | | | | 2.17-2.03 (m, 3H), |
| | | | | 2.02-1.92 (m, 3H), |
| | | | | 1.91-1.79 (m, 3H) |
| 544 | (fluoro(2- (((3S,6S,9aS)-3-(3- (5-fluoroquinolin-6- yl)azetidine-1- carbonyl)-5- oxooctahydro-1H- pyrrolo[1,2- a]azepin-6- yl)carbamoyl)benzo [b]thiophen-5- yl)methyl) phosphonic acid | 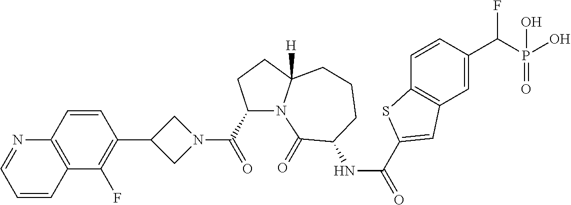 [SMILES: O=C(N[C@H]1CCC[C@H]2CC[C@@H](C(=O)N3CC(c4ccc5ncccc5c4F)C3)N2C1=O)c1cc2cc(C(F)P(=O)(O)O)ccc2s1] | 668.7 | ¹H NMR (400 MHz, DMSO-d₆) δ 9.00- 8.94 (m, 1H), 8.79 (m, 1H), 8.50 (m, 1H), 8.31-8.23 (m, 1H), 8.07-8.02 (m, 1H), 8.00 (s, 1H), 7.96-7.90 (m, 2H), 7.69-7.61 (m, 1H), 7.54 (d, J = 8.8 Hz, 1H), 5.92-5.77 (m, 1H), 4.74-4.64 (m, 2H), 4.49 (m , 2H), |
| | | | | 4.41-4.35 (m, 2H), |
| | | | | 4.06-4.02 (m, 2H), |
| | | | | 2.26-2.19 (m, 1H), |
| | | | | 2.06-1.96 (m, 2H), |
| | | | | 1.89-1.81 (m, 3H), |
| | | | | 1.79-1.67 (m, 4H) |
| 545 | ((2-(((3S,6S,9aS)-3- (3-(4-formylpyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 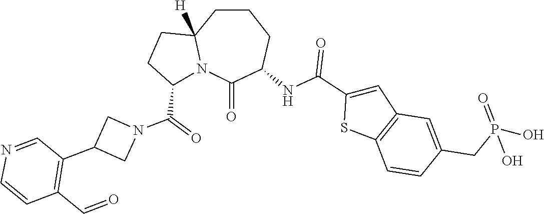 [SMILES: O=Cc1ccncc1C1CN(C(=O)[C@@H]2CC[C@@H]3CCC[C@H](NC(=O)c4cc5cc(CP(=O)(O)O)ccc5s4)C(=O)N32)C1] | 611.1 | ¹H NMR (400 MHz, DMSO-d₆) δ 10.17 (s, 1H), 8.95 (d, J = 13.2 Hz, 1H), 8.81 (dd, J = 4.8, 8.8 Hz, 1H), 8.75- 8.67 (m, 1H), 8.22- 8.12 (m, 1H), 7.95- 7.86 (m, 1H), 7.83- 7.75 (m, 2H), 7.42- 7.30 (m, 1H), 4.71- 4.26 (m, 6H), 4.09- 4.00 (m, 2H), 3.14- 3.06 (m, 2H), 2.24- |
| | | | | 2.14 (m, 1H), 2.09- |
| | | | | 1.91 (m, 2H), 1.90- |
| | | | | 1.75 (m, 4H), 1.75- |
| | | | | 1.59 (m, 3H) |
| 546 | ((2-(((3S,6S,9aS)-3- (3-(4-(1,3-dioxolan- 2-yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 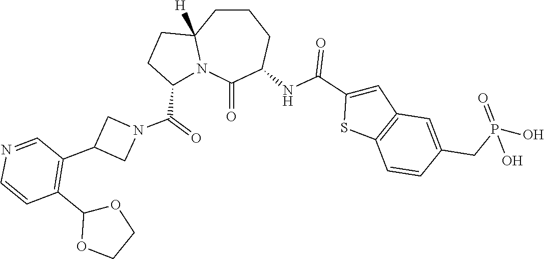 [SMILES: O=C(N[C@H]1CCC[C@H]2CC[C@@H](C(=O)N3CC(c4cnccc4C4OCCO4)C3)N2C1=O)c1cc2cc(CP(=O)(O)O)ccc2s1] | 655.2 | ¹H NMR (400 MHz, DMSO-d₆) δ 8.86- 8.65 (m, 2H), 8.53 (dd, J = 4.8, 8.8 Hz, 1H), 8.22-8.10 (m, 1H), 7.90 (dd, J = 4.8, 8.4 Hz, 1H), 7.81- 7.76 (m, 1H), 7.43 (t, J = 5.2 Hz, 1H), 7.36 (d, J = 8.4 Hz, 1H), 5.89 (d, J = 2.4 Hz, 1H), 4.79-4.54 (m, 2H), 4.50-4.43 (m, 1H), 4.34-4.22 (m, 2H), 4.22-4.10 (m, 1H), 4.06-3.93 (m, |
| | | | | 6H), 3.12 (s, 1H), |
| | | | | 3.06 (s, 1H), 2.25- |
| | | | | 2.15 (m, 1H), 2.09- |
| | | | | 1.93 (m, 2H), 1.89- |
| | | | | 1.82 (m, 2H), 1.81- |
| | | | | 1.74 (m, 2H), 1.74- |
| | | | | 1.65 (m, 2H), 1.29- |
| | | | | 1.23 (m, 1H) |
| 547 | (difluoro(2- (((3S,6S,9aS)-3-(3- methyl-3-(pyridin- 2-yl)azetidine-1- carbonyl)-5- oxooctahydro-1H- pyrrolo[1,2- a]azepin-6- yl)carbamoyl)benzo [b]thiophen-5- yl)methyl) phosphonic acid | 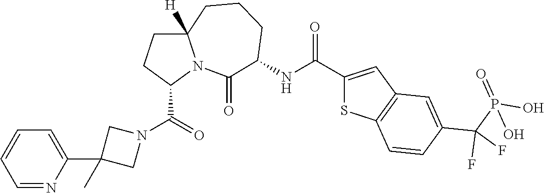 [SMILES: CC1(c2ccccn2)CN(C(=O)[C@@H]2CC[C@@H]3CCC[C@H](NC(=O)c4cc5cc(C(F)(F)P(=O)(O)O)ccc5s4)C(=O)N32)C1] | 633.2 | ¹H NMR (400 MHz, CD₃OD) δ 8.71-8.36 (m, 1H), 8.28-8.13 (m, 2H), 8.12-8.02 (m, 1H), 8.01-7.89 (m, 1H), 7.80 (dd, J = 8.0, 14.8 Hz, 1H), 7.73-7.50 (m, 2H), 4.85-4.64 (m, 2H), 4.60-4.31 (m, 3H), 4.18-3.98 (m, 2H), 2.41-2.25 (m, 1H), |
| | | | | 2.23-2.00 (m, 3H), |
| | | | | 2.00-1.78 (m, 6H), |
| | | | | 1.78-1.70 (m, 3H) |
| 548 | ((2-(((3S,6S,9aS)-3- (3-methyl-3- (pyridin-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 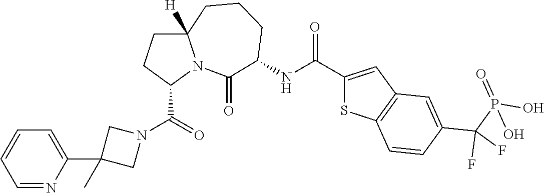 [SMILES: CC1(c2ccccn2)CN(C(=O)[C@@H]2CC[C@@H]3CCC[C@H](NC(=O)c4cc5cc(C(F)(F)P(=O)(O)O)ccc5s4)C(=O)N32)C1] | 597.1 | ¹H NMR (400 MHz, CD₃OD) δ 8.69-8.56 (m, 1H), 8.19-8.08 (m, 1H), 8.07-7.94 (m, 1H), 7.91-7.81 (m, 2H), 7.78-7.65 (m, 1H), 7.60-7.51 (m, 1H), 7.49-7.39 (m, 1H), 4.88-4.66 (m, 2H), 4.64-4.31 (m, 3H), 4.17-3.98 (m, 2H), 3.31-3.18 |
| | | | | (m,2H), 2.42-2.26 |
| | | | | (m, 1H), 2.24-2.01 |
| | | | | (m, 3H), 2.01-1.80 |
| | | | | (m, 6H), 1.77 (d, J = |
| | | | | 2.4 Hz, 3H) |
| 549 | ((2-(((3S,6S,10aS)- 3-((1- carbamoylazetidin- 3-yl)carbamoyl)-5- oxodecahydropyrrolo [1,2-a]azocin-6- yl)carbamoyl)benzo [b]thiophen-5- yl)fluoromethyl) phosphonic acid | 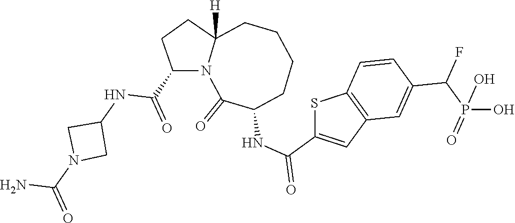 [SMILES: NC(=O)N1CC(NC(=O)[C@@H]2CC[C@@H]3CCCC[C@H](NC(=O)c4cc5cc(C(F)P(=O)(O)O)ccc5s4)C(=O)N32)C1] | 596.2 | ¹H NMR (400 MHz, CD₃OD) δ 8.10- 8.01 (m, 2H), 7.92 (d, J = 8.4 Hz, 1H), 7.59 (d, J = 8.8 Hz, 1H), 5.84-5.71 (m, 1H), 5.04-5.00 (m, 1H), 4.54-4.49 (m, 1H), 4.40-4.36 (m, 2H), 4.24-4.19 (m, 2H), 3.87-3.77 (m, 2H), 2.27-2.21 (m, 2H), 2.04-1.67 (m, 10H) |
| 550 | ((6-(((3S,6S,9aS)-3- (3-(4-ethylpyridin- 3-yl)azetidine-1- carbonyl)-5- oxooctahydro-1H- pyrrolo[1,2- a]azepin-6- yl)carbamoyl)benzo [b]thiophen-2- yl)methyl) phosphonic acid | 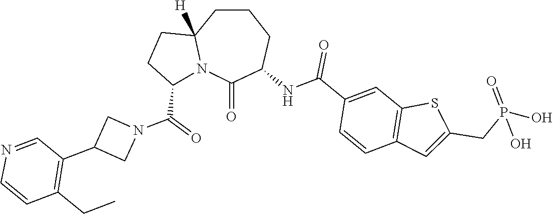 [SMILES: CCc1ccncc1C1CN(C(=O)[C@@H]2CC[C@@H]3CCC[C@H](NC(=O)c4ccc5cc(CP(=O)(O)O)sc5c4)C(=O)N32)C1] | 611.3 | ¹H NMR (400 MHz, CD₃OD) δ 8.79 (d, J = 10.8 Hz, 1H), 8.57 (dd, J = 6.0, 15.6 Hz, 1H), 8.40-8.24 (m, 1H), 7.88-7.68 (m, 3H), 7.27 (dd, J = 3.6, 6.8 Hz, 1H), 4.86- 4.69 (m, 2H), 4.67- 4.49 (m, 2H), 4.48- 4.26 (m, 2H), 4.18- 4.03 (m, 2H), 3.50- 3.37 (m, 2H), 2.91- |
| | | | | 2.72 (m, 2H), 2.42- |
| | | | | 2.27 (m, 1H), 2.23- |
| | | | | 1.95 (m, 5H), 1.95- |
| | | | | 1.76 (m, 4H), 1.37- |
| | | | | 1.22 (m, 3H) |
| 551 | ((2-(((4S,7S,10aS)- 4-((rel-trans)-3- cyano-4- phenylpyrrolidine- 1-carbonyl)-6- oxodecahydropyrido [1,2-a]azepin-7- yl)carbamoyl)benzo [b]thiophen-5- yl)fluoromethyl) phosphonic acid | 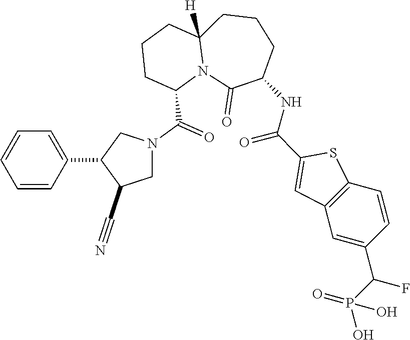 [SMILES: N#C[C@@H]1CN(C(=O)[C@@H]2CCC[C@@H]3CCC[C@H](NC(=O)c4cc5cc(C(F)P(=O)(O)O)ccc5s4)C(=O)N32)C[C@H]1c1ccccc1] | 653.3 | ¹H NMR (400 MHz, CD₃OD) δ 8.13-8.02 (m, 2H), 7.94 (dd, J = 3.2, 8.4 Hz, 1H), 7.60 (d, J = 8.8 Hz, 1H), 7.50-7.28 (m, 5H), 5.91-5.68 (m, 1H), 5.09 (dd, J = 4.4, 9.2 Hz, 1H), 5.01-4.97 (m, 1H), 4.18 (d, J = 10.0 Hz, 1H), 4.12- 3.99 (m, 1H), 3.96- 3.67 (m, 2H), 3.66- 3.52 (m, 2H), 3.51- 3.39 (m, 1H), 2.56- 2.40 (m, 1H), 2.11- 2.01 (m, 2H), 2.00- 1.86 (m, 4H), 1.82- 1.68 (m, 3H), 1.64- 1.48 (m, 2H) |
| 552 | ((2-(((4S,7S,11aS)- 4-((rel-trans)-3- cyano-4- phenylpyrrolidine- 1-carbonyl)-6- oxodecahydro-2H- pyrido[1,2- a]azocin-7- yl)carbamoyl)benzo [b]thiophen-5- yl)fluoromethyl) phosphonic acid | 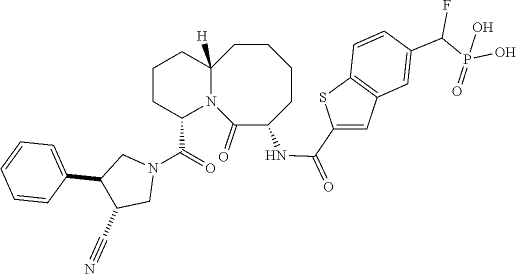 [SMILES: N#C[C@H]1CN(C(=O)[C@@H]2CCC[C@@H]3CCCC[C@H](NC(=O)c4cc5cc(C(F)P(=O)(O)O)ccc5s4)C(=O)N32)C[C@@H]1c1ccccc1] | 667.0 | ¹H NMR (400 MHz, DMSO-d₆) δ 8.86- 8.69 (m, 1H), 8.33- 8.23 (m, 1H), 8.09- 7.91 (m, 2H), 7.56- 7.48 (m, 1H), 7.48- 7.36 (m, 4H), 7.35- 7.28 (m, 1H), 5.95- 5.71 (m, 1H), 5.09- 4.98 (m, 1H), 4.93- 4.81 (m, 1H), 4.41- 4.22 (m, 2H), 4.20- 4.06 (m, 1H), 4.04- 3.84 (m, 2H), 3.83- 3.77 (m, 1H), 3.24 (d, J = 10.8 Hz, 1H), 1.95- 1.73 (m, 6H), 1.73- |
| | | | | 1.55 (m, 4H), 1.55- |
| | | | | 1.40 (m, 3H), 1.39- |
| | | | | 1.27 (m, 1H) |
| 553 | ((2- (((1R,3S,6S,11aS)- 3-((rel-trans)-3- cyano-4- phenylpyrrolidine- 1-carbonyl)-1- hydroxy-5- oxodecahydro-1H- pyrrolo[1,2- a]azonin-6- yl)carbamoyl)benzo [b]thiophen-5- yl)fluoromethyl) phosphonic acid | 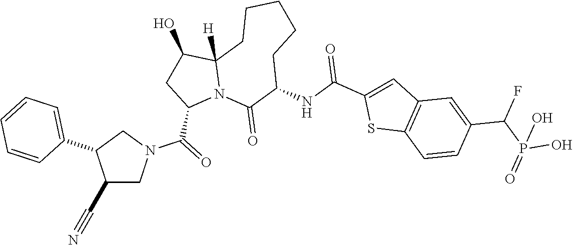 [SMILES: N#C[C@@H]1CN(C(=O)[C@@H]2C[C@@H](O)[C@@H]3CCCCC[C@H](NC(=O)c4cc5cc(C(F)P(=O)(O)O)ccc5s4)C(=O)N23)C[C@H]1c1ccccc1] | 683.2 | ¹H NMR (400 MHz, CD₃OD) δ 8.12-8.07 (m, 1H), 8.02 (s, 1H), 7.97-7.88 (m, 1H), 7.62-7.56 (m, 1H), 7.46-7.27 (m, 5H), 5.91-5.71 (m, 1H), 5.19-5.10 (m, 1H), 4.92 (s, 1H), 4.24- 4.12 (m, 3H), 4.11- 3.74 (m, 2H), 3.73- 3.39 (m, 3H), 2.30- 2.18 (m, 1H), 2.16- 2.02 (m, 3H), 2.02- 1.89 (m, 2H), 1.88- |
| | | | | 1.72 (m, 3H), 1.70- |
| | | | | 1.50 (m, 3H) |
| 554 | ((2-(((3S,6S,10aS)- 3-(((3S,4S)-1- (cyclopropoxy- carbonyl)-4- fluoropyrrolidin-3- yl)carbamoyl)-5- oxodecahydropyrrolo [1,2-a]azocin-6- yl)carbamoyl)benzo [b]thiophen-5- yl)fluoromethyl) phosphonic acid | 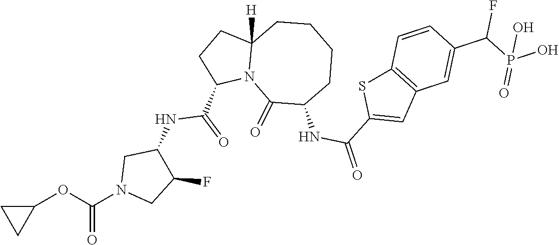 [SMILES: O=C(N[C@H]1CCCC[C@H]2CC[C@@H](C(=O)N[C@H]3CN(C(=O)OC4CC4)C[C@@H]3F)N2C1=O)c1cc2cc(C(F)P(=O)(O)O)ccc2s1] | 669.0 | ¹H NMR (400 MHz, CD₃OD) δ 8.10 (s, 1H), 8.01 (s, 1H), 7.94 (d, J = 8.4 Hz, 1H), 7.58 (d, J = 8.4 Hz, 1H), 5.91-5.73 (m, 1H), 5.05-5.00 (m, 1H), 4.92 (s, 1H), 4.45-4.31 (m, 3H), 4.05-4.00 (m, 1H), 3.68 (s, 1H), 3.61 (d, J = 6.0 Hz, 1H), 3.57- 3.52 (m, 1H), 3.50- 3.39 (m, 1H), 2.26- 2.16 (m, 2H), 2.07- |
| | | | | 1.92 (m, 6H), 1.84- |
| | | | | 1.80 (m, 2H), 1.73- |
| | | | | 1.58 (m, 2H), 0.67 (s, |
| | | | | 4H |
| 555 | (fluoro(2- (((3S,6S,10aS)-3- (((3S,4S)-4-fluoro- 1-(3-methylpyridin- 2-yl)pyrrolidin-3- yl)carbamoyl)-5- oxodecahydropyrrolo [1,2-a]azocin-6- yl)carbamoyl)benzo [b]thiophen-5- yl)methyl) phosphonic acid | 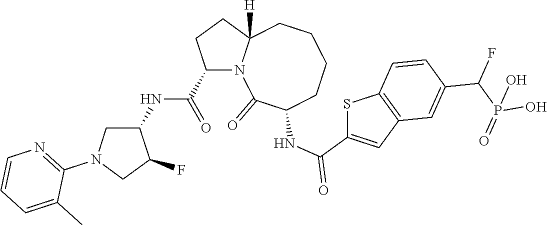 [SMILES: Cc1cccnc1N1C[C@H](NC(=O)[C@@H]2CC[C@@H]3CCCC[C@H](NC(=O)c4cc5cc(C(F)P(=O)(O)O)ccc5s4)C(=O)N32)[C@@H](F)C1] | 676.0 | ¹H NMR (400 MHz, CD₃OD) δ 7.96-7.83 (m, 2H), 7.73 (d, J = 4.0 Hz, 1H), 7.66- 7.46 (m, 3H), 6.70 (q, J = 7.2 Hz, 1H), 5.83- 5.65 (m, 1H), 5.32- 5.14 (m, 1H), 4.97 ( d, J = 6.0 Hz, 1H), 4.65- 4.61 (m, 1H), 4.46- 4.17 (m, 4H), 4.07- 3.84 (m, 2H), 2.48- 2.39 (m, 3H), 2.36- 2.17 (m, 2H), 2.14- |
| | | | | 1.90 (m, 6H), 1.87- |
| | | | | 1.83 (m, 2H), 1.76- |
| | | | | 1.57 (m, 2H) |
| 556 | ((2-(((3S,6S,9aS)-3- (3-(4-fluoro-6- methoxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 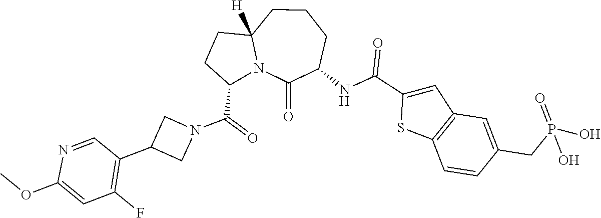 [SMILES: COc1cc(F)c(C2CN(C(=O)[C@@H]3CC[C@@H]4CCC[C@H](NC(=O)c5cc6cc(CP(=O)(O)O)ccc6s5)C(=O)N43)C2)cn1] | 631.5 | ¹H NMR (400 MHz, CD₃OD) δ 8.30-8.10 (m, 1H), 8.02 (d, J = 3.2 Hz, 1H), 7.91- 7.75 (m, 2H), 7.43 (d, J = 6.8 Hz, 1H), 6.61 (d, J = 11.6 Hz, 1H), 4.75 (d, J = 11.2 Hz, 1H), 4.65 (d, J = 8.0 Hz, 1H), 4.57 (d, J = 3.6 Hz, 1H), 4.48- 4.34 (m, 2H), 4.17- 4.03 (m, 3H), 3.96- |
| | | | | 3.83 (m, 3H), 3.28 (d, |
| | | | | J = 2.4 Hz, 1H), 3.23 |
| | | | | (s, 1H), 2.40-2.21 |
| | | | | (m, 1H), 2.18-1.62 |
| | | | | (m, 9H) |
| 557 | ((2-(((3S,6S,9aS)-3- (3-(4-cyanopyridin- 3-yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 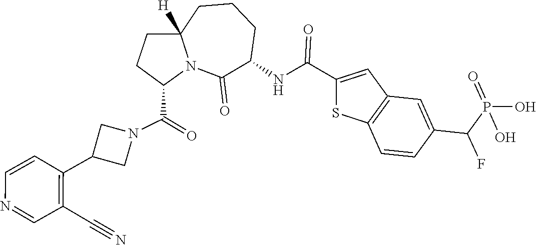 [SMILES: N#Cc1cnccc1C1CN(C(=O)[C@@H]2CC[C@@H]3CCC[C@H](NC(=O)c4cc5cc(C(F)P(=O)(O)O)ccc5s4)C(=O)N32)C1] | 626.1 | ¹H NMR (400 MHz, CD₃OD) δ 9.18-8.87 (m, 1H), 8.69 (d, J = 4.4 Hz, 1H), 8.14- 8.03 (m, 2H), 7.99- 7.92 (m, 1H), 7.74 (dd, J = 4.8, 18.8 Hz, 1H), 7.59 (dd, J = 1.6, 8.4 Hz, 1H), 5.94- 5.72 (m, 1H), 4.80- 4.74 (m, 2H), 4.68- 4.44 (m, 3H), 4.41- 4.31 (m, 1H), 4.23 (dd, J = 6.4, 9.2 Hz, 1H), 4.13-4.03 (m, |
| | | | | 1H), 2.38-2.25 (m, |
| | | | | 1H), 2.20-1.99 (m, |
| | | | | 5H), 1.95-1.78 (m, |
| | | | | 4H) |
| 558 | ((2-(((3S,6S,10aS)- 3-(((3S,4S)-1- carbamoyl-4- fluoropyrrolidin-3- yl)carbamoyl)-5- oxodecahydropyrrolo [1,2-a]azocin-6- yl)carbamoyl)benzo [b]thiophen-5- yl)fluoromethyl) phosphonic acid | 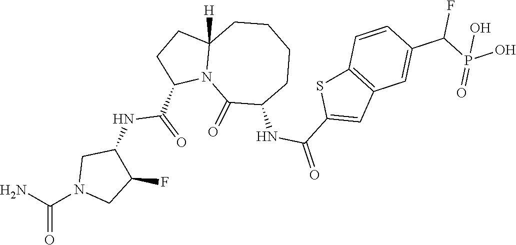 [SMILES: NC(=O)N1C[C@H](NC(=O)[C@@H]2CC[C@@H]3CCCC[C@H](NC(=O)c4cc5cc(C(F)P(=O)(O)O)ccc5s4)C(=O)N32)[C@@H](F)C1] | 628.0 | ¹H NMR (400 MHz, DMSO-d6) δ 8.86- 8.79 (m, 1H), 8.38 (d, J = 7.2 Hz, 1H), 8.26 (s, 1H), 8.03 (d, J = 8.4 Hz, 1H), 7.96 (s, 1H), 7.52 (d, J = 8.4 Hz, 1H), 5.91 (s, 1H), 5.91-5.89 (m, 1H), 5.89-5.74 (m, 1H), 5.03-4.88 (m, 2H), 4.31-4.22 (m, 3H), 3.53 (s, 2H), 3.45 (m, 1H), 3.28-3.23 (m, 1H), 2.12-2.00 (m, |
| | | | | 2H), 1.99-1.77 (m, |
| | | | | 6H), 1.75-1.63 (m, |
| | | | | 2H), 1.62-1.48 (m, |
| | | | | 2H) |
| 559 | ((2-(((3S,6S,10aS)- 3-(((R)-1-(1H- pyrazol-5- yl)pyrrolidin-3- yl)carbamoyl)-5- oxodecahydropyrrolo [1,2-a]azocin-6- yl)carbamoyl)benzo [b]thiophen-5- yl)fluoromethyl) phosphonic acid | 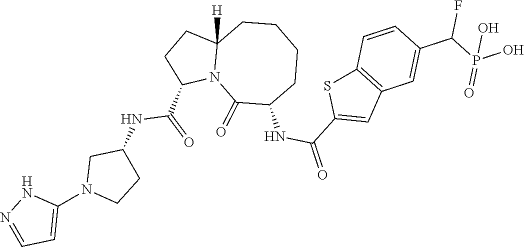 [SMILES: O=C(N[C@H]1CCCC[C@H]2CC[C@@H](C(=O)N[C@@H]3CCN(c4ccn[nH]4)C3)N2C1=O)c1cc2cc(C(F)P(=O)(O)O)ccc2s1] | 633.6 | ¹H NMR (400 MHz, DMSO-d6) δ 8.84 (d, J = 7.2 Hz, 1H), 8.29- 8.24 (m, 2H), 8.03 (d, J = 8.4 Hz, 1H), 7.96 (s, 1H), 7.54- 7.46 (m, 2H), 5.90- 5.74 (m, 1H), 5.52 (d, J = 2.0 Hz, 1H), 4.96- 4.89 (m, 1H), 4.29- 4.23 (m, 2H), 3.42- 3.39 (m, 1H), 3.33- 3.28 (m, 1H), 3.17 (d, J = 6.0 Hz, 1H), 3.01 (dd, J = 4.4, 10.0 Hz, |
| | | | | 1H), 2.17-2.03 (m, |
| | | | | 3H), 1.89-1.76 (m, |
| | | | | 6H), 1.72-1.45 (m, |
| | | | | 5H) |
| 560 | (fluoro(2- (((3S,6S,9aS)-3-(3- (7-fluoroquinolin-6- yl)azetidine-1- carbonyl)-5- oxooctahydro-1H- pyrrolo[1,2- a]azepin-6- yl)carbamoyl)benzo [b]thiophen-5- yl)methyl) phosphonic acid | 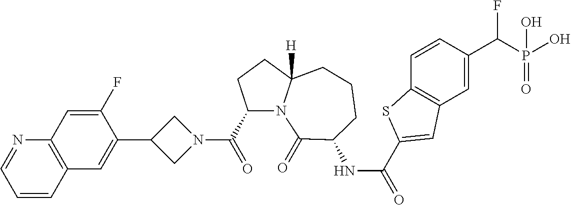 [SMILES: O=C(N[C@H]1CCC[C@H]2CC[C@@H](C(=O)N3CC(c4cc5cccnc5cc4F)C3)N2C1=O)c1cc2cc(C(F)P(=O)(O)O)ccc2s1] | 668.9 | 1H NMR (400 MHz, CD₃OD) δ 8.60-8.37 (m, 2H), 8.17 (s, 1H), 7.91-7.52 (m, 5H), 7.30-7.20 (m, 1H), 5.90-5.72 (m, 1H), 5.02-4.90 (m, 1H), 4.79-4.68 (m, 2H), 4.61-4.47 (m, 2H), 4.40-4.24 (m, 1H), 4.20-4.03 (m, 2H), 2.39-2.26 (m, 1H), 2.20-2.02 (m, 3H), |
| | | | | 2.01-1.87 (m, 4H), |
| | | | | 1.86-1.74 (m, 2H) |
| 561 | ((2-(((3S,6S,9aS)-3- (3-(4-fluoro-2- methoxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 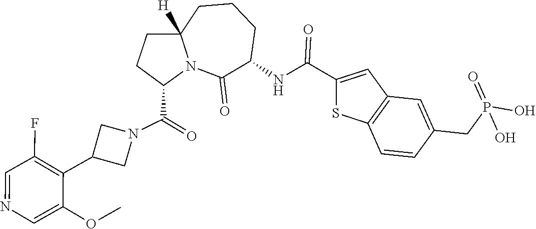 [SMILES: COc1cncc(F)c1C1CN(C(=O)[C@@H]2CC[C@@H]3CCC[C@H](NC(=O)c4cc5cc(CP(=O)(O)O)ccc5s4)C(=O)N32)C1] | 631.5 | ¹H NMR (400 MHz, CD₃OD) δ 8.07-7.98 (m, 2H), 7.85-7.82 (m, 2H), 7.43-7.41 (m, 1H), 6.83-6.75 (m, 1H), 4.77-4.71 (m, 2H), 4.60-4.55 (m, 2H), 4.29-4.23 (m, 3H), 4.00-3.98 (m, 4H), 3.28-3.22 (m, 2H), 2.31-2.13 (m, 1H), 2.00-1.83 (m, 9H) |
| 562 | ((2-(((3S,6S,9aS)-3- (3-(2- (difluoromethyl) pyridin-3-yl)azetidine- 1-carbonyl)-5- oxooctahydro-1H- pyrrolo[1,2- a]azepin-6- yl)carbamoyl)benzo [b]thiophen-5- yl)methyl) phosphonic acid | 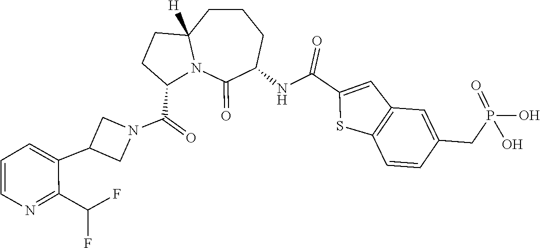 [SMILES: O=C(N[C@H]1CCC[C@H]2CC[C@@H](C(=O)N3CC(c4cccnc4C(F)F)C3)N2C1=O)c1cc2cc(CP(=O)(O)O)ccc2s1] | 632.9 | 1H NMR (400 MHz, CD₃OD) δ 8.57-8.45 (m, 1H), 8.41 (d, J = 4.0 Hz, 1H), 8.02 (s, 1H), 7.89-7.81 (m, 2H), 7.67-7.52 (m, 1H), 7.47-7.40 (m, 1H), 6.95-6.81 (m, 1H), 4.96-4.90 (m, 1H), 4.75 (d, J = 24.0 Hz, 1H), 4.71-4.53 (m, 2H), 4.50-4.44 (m, 1H), 4.42-4.29 (m, 1H), 4.12-4.01 (m, 2H), 3.29 (s, 1H), |
| | | | | 3.23 (s, 1H), 2.40- |
| | | | | 2.24 (m, 1H), 2.20- |
| | | | | 2.06 (m, 2H), 2.05- |
| | | | | 1.93 (m, 3H), 1.93- |
| | | | | 1.76 (m, 4H) |
| 563 | (fluoro(2- (((3S,6S,10aS)-3-(3- (2-fluorophenyl) pyrrolidine-1- carbonyl)-5- oxodecahydropyrrolo [1,2-a]azocin-6- yl)carbamoyl)benzo [b]thiophen-5- yl)methyl) phosphonic acid | 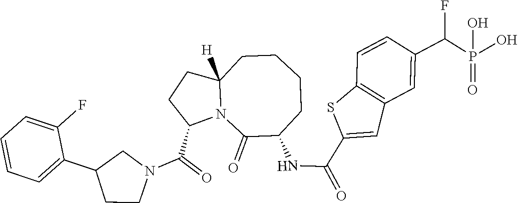 [SMILES: O=C(N[C@H]1CCCC[C@H]2CC[C@@H](C(=O)N3CCC(c4ccccc4F)C3)N2C1=O)c1cc2cc(C(F)P(=O)(O)O)ccc2s1] | 646.2 | 1H NMR (400 MHz, CD₃OD) δ 8.14-8.06 (m, 1H), 8.00 (s, 1H), 7.95-7.88 (m, 1H), 7.57 (d, J = 8.4 Hz, 1H), 7.42-7.19 (m, 2H), 7.18-6.99 (m, 2H), 5.94-5.70 (m, 1H), 5.05 (t, J = 8.8 Hz, 1H), 4.77-4.67 (m, 1H), 4.44 (m, 1H), 4.12-3.94 (m, |
| | | | | 1H), 3.93-3.81 (m, |
| | | | | 1H), 3.80-3.71 (m, |
| | | | | 1H), 3.64 (m, 1H), |
| | | | | 3.57 (s, 1H), 2.40- |
| | | | | 2.14 (m, 4H), 2.11- |
| | | | | 1.93 (m, 6H), 1.90- |
| | | | | 1.74 (m, 2H), 1.72- |
| | | | | 1.54 (m, 2H) |
| 564 | (fluoro(2- (((3S,6S,10aS)-5- oxo-3-(4-(pyridin-3- yl)piperidine-1- carbonyl)decahydro pyrrolo[1,2- a]azocin-6- yl)carbamoyl)benzo [b]thiophen-5- yl)methyl) phosphonic acid | 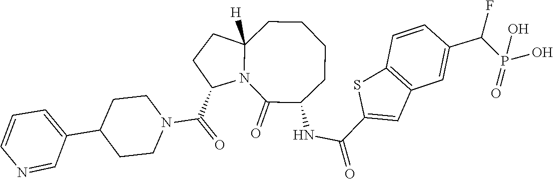 [SMILES: O=C(N[C@H]1CCCC[C@H]2CC[C@@H](C(=O)N3CCC(c4cccnc4)CC3)N2C1=O)c1cc2cc(C(F)P(=O)(O)O)ccc2s1] | 643.3 | ¹H NMR (400 MHz, CD₃OD) δ 8.62-8.44 (m, 2H), 8.41 ( d, J = 8.0 Hz, 1H), 8.06- 7.99 (m, 1H), 7.98- 7.91 (m, 1H), 7.90- 7.74 (m, 2H), 7.66- 7.51 (m, 1H), 5.90- 5.65 (m, 1H), 5.10- 4.90 (m, 3H), 4.70- 4.62 (m, 1H), 4.52- |
| | | | | 4.41 (m, 1H), 4.36- |
| | | | | 4.23 (m, 1H), 3.17- |
| | | | | 3.01 (m, 1H), 2.87- |
| | | | | 2.80 ( m, 1H), 2.43- |
| | | | | 2.15 (m, 3H), 2.13- |
| | | | | 1.93 (m, 6H), 1.92- |
| | | | | 1.80 (m, 4H), 1.76- |
| | | | | 1.56 (m, 3H) |
| 565 | (fluoro(2- (((3S,6S,9aS)-3-(3-(2- fluorophenyl) azetidine-1- carbonyl)-5- oxooctahydro-1H- pyrrolo[1,2- a]azepin-6- yl)carbamoyl)benzo [b]thiophen-5- yl)methyl) phosphonic acid | 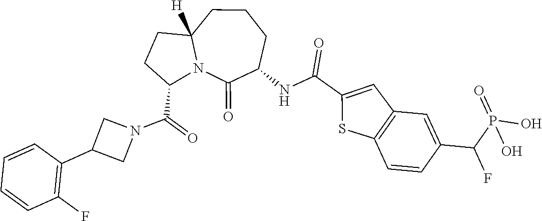 [SMILES: O=C(N[C@H]1CCC[C@H]2CC[C@@H](C(=O)N3CC(c4ccccc4F)C3)N2C1=O)c1cc2cc(C(F)P(=O)(O)O)ccc2s1] | 618.3 | ¹H NMR (400 MHz, CD₃OD) δ 8.09-7.99 (m, 2H), 7.94 (t, J = 8.0 Hz, 1H), 7.61- 7.39 (m, 2H), 7.34- 7.23 (m, 1H), 7.23- 7.14 (m, 1H), 7.14- 7.02 (m, 1H), 5.90- 5.74 (m, 1H), 4.94- 4.90 (m, 1H), 4.77- 4.71 (m, 1H), 4.69- 4.55 (m, 2H), 4.41- 4.32 (m, 1H), 4.26- 4.13 (m, 1H), 4.12- |
| | | | | 3.99 (m, 2H), 2.35- |
| | | | | 2.26 (m, 1H), 2.18- |
| | | | | 2.05 (m, 2H), 2.03- |
| | | | | 1.77 (m, 7H) |
| 566 | ((2-(((3S,6S,10aS)- 3-(3-(4- fluoropyridin-3- yl)azetidine-1- carbonyl)-5- oxodecahydropyrrolo [1,2-a]azocin-6- yl)carbamoyl)benzo [b]thiophen-5- yl)methyl) phosphonic acid | 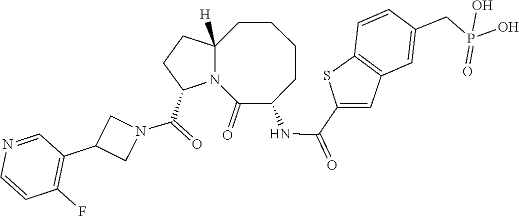 [SMILES: O=C(N[C@H]1CCCC[C@H]2CC[C@@H](C(=O)N3CC(c4cnccc4F)C3)N2C1=O)c1cc2cc(CP(=O)(O)O)ccc2s1] | 615.3 | ¹H NMR (400 MHz, CD₃OD) δ 8.78-8.58 (m, 1H), 8.54-8.42 (m, 1H), 8.08-7.99 (m, 1H), 7.85-7.77 (m, 2H), 7.44-7.38 (m, 1H), 7.31-7.19 (m, 1H), 5.06-4.99 (m, 1H), 4.99-4.89 (m, 1H), 4.76-4.66 (m, 1H), 4.52-4.46 (m, 1H), 4.45-4.36 (m, 2H), 4.20 (q, J = |
| | | | | 6.4 Hz, 1H), 4.17- |
| | | | | 4.09 (m, 1H), 3.26- |
| | | | | 3.18 (m, 2H), 2.32- |
| | | | | 2.19 (m, 2H), 2.08- |
| | | | | 1.92 (m, 6H), 1.89- |
| | | | | 1.77 (m, 2H), 1.73- |
| | | | | 1.60 (m, 2H) |
| 567 | (fluoro(2-(((3S, 6S,10aS)-3-(3-(2- methoxyacetamido)- 3-(pyridin-2- yl)azetidine-1- carbonyl)-5- oxodecahydropyrrolo [1,2-a]azocin-6- yl)carbamoyl)benzo [b]thiophen-5- yl)methyl) phosphonic acid | 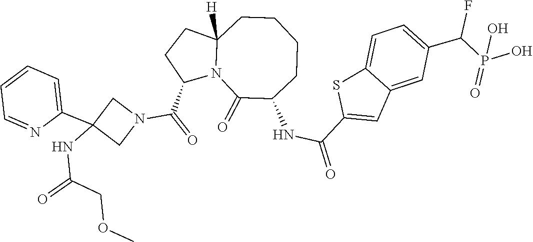 [SMILES: COCC(=O)NC1(c2ccccn2)CN(C(=O)[C@@H]2CC[C@@H]3CCCC[C@H](NC(=O)c4cc5cc(C(F)P(=O)(O)O)ccc5s4)C(=O)N32)C1] | 702.4 | ¹H NMR (400 MHz, CD₃OD) δ 8.65-8.46 (m, 1H), 8.10-8.03 (m, 1H), 7.99 (d, J = 5.6 Hz, 1H), 7.92 (s, 1H), 7.83-7.73 (m, 1H), 7.59 (d, J = 7.2 Hz, 1H), 7.44 (d, J = 8.0 Hz, 1H), 7.32 (dd, J = 4.4, 7.1 Hz, 1H), 5.90-5.71 (m, 1H), 5.02 (dd, J = 5.6, 10.6 Hz, 1H), 4.95 ( d, J = 9.2 Hz, 1H), 4.80- 4.76 (m, 1H), 4.54- |
| | | | | 4.45 (m, 2H), 4.44- |
| | | | | 4.35 (m, 2H), 3.95 ( d, |
| | | | | J = 12.8 Hz, 2H), 3.47 |
| | | | | (s, 3H), 2.35-2.21 |
| | | | | (m, 2H), 2.10-1.94 |
| | | | | (m, 6H), 1.90-1.78 |
| | | | | (m, 2H), 1.73-1.59 |
| | | | | (m, 2H) |
| 568 | (fluoro(2- (((3S,6S,10aS)-3-(3- ((2- methoxyethyl) amino)-3- (pyridin-2- yl)azetidine-1- carbonyl)-5- oxodecahydropyrrolo [1,2-a]azocin-6- yl)carbamoyl)benzo [b]thiophen-5- yl)methyl) phosphonic acid | 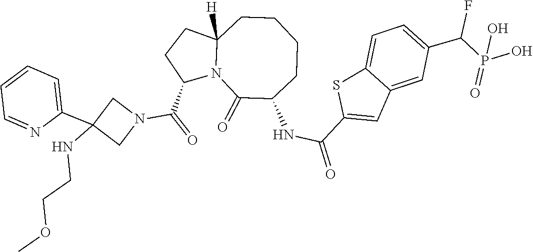 [SMILES: COCCNC1(c2ccccn2)CN(C(=O)[C@@H]2CC[C@@H]3CCCC[C@H](NC(=O)c4cc5cc(C(F)P(=O)(O)O)ccc5s4)C(=O)N32)C1] | 688.2 | ¹H NMR (400 MHz, DMSO-d₆) δ 8.87- 8.76 (m, 1H), 8.59 (dd, J = 4.8, 11.2 Hz, 1H), 8.31-8.18 (m, 1H), 8.00 (t, J = 8.0 Hz, 1H), 7.94 (d, J = 14.4 Hz, 1H), 7.90- 7.81 (m, 1H), 7.63 (dd, J = 7.6, 16 Hz, 1H), 7.49 (d, J = 6.0 Hz, 1H), 7.40-7.31 (m, 1H), 5.77-5.61 (m, 1H), 4.93 ( dd, J = 4.8, 11.0 Hz, 1H), |
| | | | | 4.75-4.56 (m, 1H), |
| | | | | 4.48 (d, J = 7.6 Hz, |
| | | | | 1H), 4.35-4.18 (m, |
| | | | | 4H), 4.04-3.95 (m, |
| | | | | 2H), 3.35 (d, J = 4.8 |
| | | | | Hz, 2H), 3.19 (d, J = |
| | | | | 7.6 Hz, 3H), 2.48- |
| | | | | 2.44 (m, 1H), 2.23- |
| | | | | 2.01 (m, 2H), 1.94- |
| | | | | 1.77 (m, 6H), 1.76- |
| | | | | 1.65 (m, 2H), 1.63- |
| | | | | 1.46 (m, 2H) |
| 569 | (fluoro(2- (((3S,6S,10aS)-3-(6- (1-methyl-2-oxo- 1,2-dihydropyridin- 4-y1)-5,7-dioxo-4,6- diazaspiro[2.4] heptane-4- carbonyl)-5- oxodecahydropyrrolo [1,2-a]azocin-6- yl)carbamoyl)benzo [b]thiophen-5- | 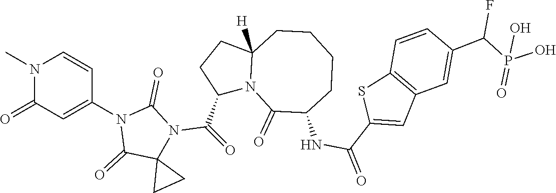 [SMILES: Cn1ccc(N2C(=O)N(C(=O)[C@@H]3CC[C@@H]4CCCC[C@H](NC(=O)c5cc6cc(C(F)P(=O)(O)O)ccc6s5)C(=O)N43)C3(CC3)C2=O)cc1=O] | 714.1 | ¹H NMR (400 MHz, DMSO-d₆) δ 8.88 (d, J = 7.2 Hz, 1H), 8.26 (s, 1H), 8.04 (d, J = 8.4 Hz, 1H), 7.97 (s, 1H), 7.83 (d, J = 7.2 Hz, 1H), 7.53 (d, J = 8.4 Hz, 1H), 6.56 (d, J = 2.0 Hz, 1H), 6.42 (dd, J = 2.0, 7.2 Hz, 1H), 5.92-5.71 (m, 1H), 5.35 (t, J = 8.4 |
| | yl)methyl) | | | Hz, 1H), 4.94 (d, J = |
| | phosphonic acid | | | 6.4 Hz, 1H), 4.41- |
| | | | | 4.30 (m, 1H), 3.44 (s, |
| | | | | 3H), 2.42 (d, J = 5.6 |
| | | | | Hz, 1H), 2.24-2.16 |
| | | | | (m, 1H), 2.11-2.04 |
| | | | | (m, 1H), 1.95-1.82 |
| | | | | (m, 5H), 1.80-1.65 |
| | | | | (m, 4H), 1.60-1.49 |
| | | | | (m, 1H), 1.36 (d, J = |
| | | | | 4.4 Hz, 2H), 1.29- |
| | | | | 1.21 (m, 1H) |
| 570 | ((6-(((3S,6S,10aS)- 5-oxo-3-(6-phenyl- 4-azaspiro[2.4] heptane-4- carbonyl)decahydro pyrrolo[1,2- a]azocin-6- yl)carbamoyl) isoquinolin-3- yl)methyl) phosphonic acid | 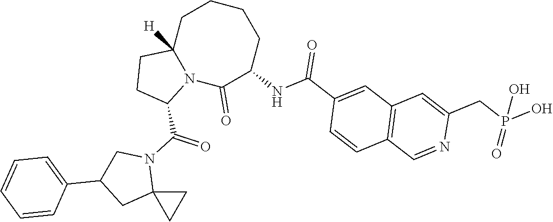 [SMILES: O=C(N[C@H]1CCCC[C@H]2CC[C@@H](C(=O)N3CC(c4ccccc4)CC34CC4)N2C1=O)c1ccc2cnc(CP(=O)(O)O)cc2c1] | 631.2 | ¹H NMR (400 MHz, CD₃OD) δ 9.49 (d, J = 8.0 Hz, 1H), 8.59- 8.47 (m, 1H), 8.29- 8.19 (m, 2H), 8.18- 8.09 (m, 1H), 7.28 (d, J = 7.6 Hz, 1H), 7.25 (d, J = 4.4 Hz, 2H), 7.22-7.06 (m, 2H), 5.16-5.04 (m, 1H), 4.76-4.64 (m, 1H), 4.56-4.38 (m, 2H), 4.19-3.90 (m, 1H), |
| | | | | 3.65-3.52 (m, 3H), |
| | | | | 2.42-2.16 (m, 4H), |
| | | | | 2.08-1.93 (m, 7H), |
| | | | | 1.90-1.76 (m, 3H), |
| | | | | 1.69-1.60 (m, 2H), |
| | | | | 0.60-0.50 (m, 2H) |
| 571 | ((7-(((3S,6S,10aS)- 5-oxo-3-(6-phenyl- 4-azaspiro[2.4] heptane-4- carbonyl)decahydro pyrrolo[1,2- a]azocin-6- yl)carbamoyl) quinolin-2- yl)methyl) phosphonic acid | 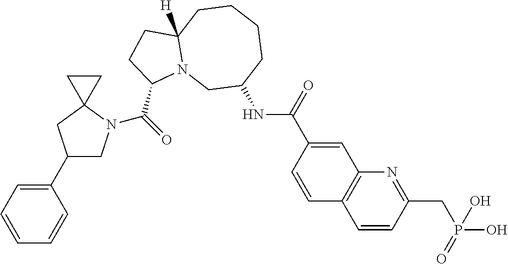 | 631.2 | ¹H NMR (400 MHz, CD₃OD) δ 8.75 (d, J = 4.8 Hz, 1H), 8.41 (d, J = 14.8 Hz, 1H), 8.20-8.05 (m, 2H), 7.97 (d, J = 8.4 Hz, 1H), 7.42-7.03 (m, 5H), 5.10-5.05 (m, 1H), 4.75-4.68 (m, 2H), 4.56-4.39 (m, 2H), 4.16-3.93 (m, 1H), 3.75-3.51 (m, 2H), 2.43-2.18 (m, 4H), 2.17-1.96 (m, 7H), 1.90-1.75 (m, 3H), 1.71-1.59 (m, 2H), 0.66-0.44 (m, 2H) |
| 572 | ((2-(((3S,6S,10aS)- 3-(6-(1- (difluoromethyl)-2- oxo-1,2- dihydropyridin-4- yl)-5,7-dioxo-4,6- diazaspiro[2.4] heptane-4- carbonyl)-5- oxodecahydropyrrolo [1,2-a]azocin-6- yl)carbamoyl)benzo [b]thiophen-5- yl)fluoromethyl) | 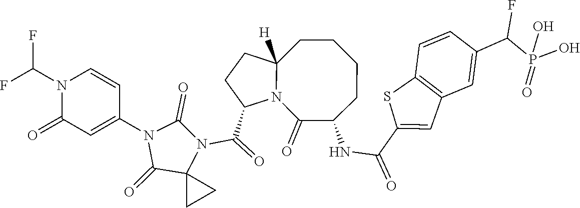 [SMILES: O=C(N[C@H]1CCCC[C@H]2CC[C@@H](C(=O)N3C(=O)N(c4ccn(C(F)F)c(=O)c4)C(=O)C34CC4)N2C1=O)c1cc2cc(C(F)P(=O)(O)O)ccc2s1] | 750.1 | ¹H NMR (400 MHz, DMSO-d₆) δ 8.88 (d, J = 6.8 Hz, 1H), 8.25 (s, 1H), 8.04-7.94 (m, 3H), 7.92-7.65 (m, 1H), 7.51 (d, J = 8.0 Hz, 1H), 6.71 (s, 1H), 6.69-6.64 (m, 1H), 5.86-5.68 (m, 1H), 5.34 (t, J = 8.8 Hz, 1H), 4.97-4.89 (m, 1H), 4.36 (t, J = 8.8 Hz, 1H), 2.47- 2.41 (m, 1H), 2.36- |
| | phosphonic acid | | | 2.32 (m, 1H), 2.23- |
| | | | | 2.15 (m, 1H), 2.10- |
| | | | | 2.05 (m, 1H), 1.94- |
| | | | | 1.83 (m, 5H), 1.80- |
| | | | | 1.66 (m, 4H), 1.60- |
| | | | | 1.51 (m, 1H), 1.42- |
| | | | | 1.34 (m, 2H) |
| 573 | ((2-(((3S,6S,10aS)- 3-(3-(1,1- dioxidothio- morpholino) azetidine-1- carbonyl)-5- oxodecahydropyrrolo [1,2-a]azocin-6- yl)carbamoyl)benzo [b]thiophen-5- yl)fluoromethyl) | 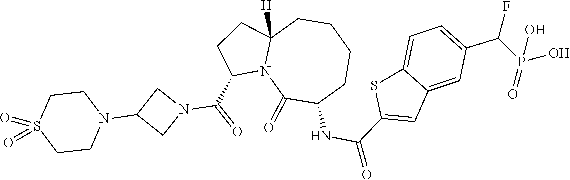 [SMILES: O=C(N[C@H]1CCCC[C@H]2CC[C@@H](C(=O)N3CC(N4CCS(=O)(=O)CC4)C3)N2C1=O)c1cc2cc(C(F)P(=O)(O)O)ccc2s1] | 671.1 | ¹H NMR (400 MHz, DMSO-d₆) δ 8.86 8.81 (m, 1H), 8.31- 8.11 (m, 1H), 8.02- 7.77 (m, 2H), 7.52- 7.49 (m, 1H), 7.39- 7.20 (m, 1H), 5.56- 5.36 (m, 1H), 4.91 (d, J = 7.2 Hz, 1H), 4.30- 4.17 (m, 3H), 4.02- 3.90 (m, 2H), 3.82- |
| | phosphonic acid | | | 3.78 (m, 1H), 3.70 (s, |
| | | | | 1H), 3.65-3.62 (m, |
| | | | | 1H), 3.40 (d, J = 6.4 |
| | | | | Hz, 1H), 3.13-3.07 |
| | | | | (m, 3H), 2.81-2.72 |
| | | | | (m, 3H), 2.19-2.09 |
| | | | | (m, 1H), 2.09-1.97 |
| | | | | (m, 1H), 1.97-1.68 |
| | | | | (m, 7H), 1.68-1.41 |
| | | | | (m, 3H) |
| 574 | ((2-(((3S,6S,9aS)-5- oxo-3-(3-(pyridin-3- yl)azetidine-1- carbonyl)octahydro- 1H-pyrrolo[1,2- a]azepin-6- yl)carbamoyl)thieno [2,3-c]pyridin-5- yl)methyl) phosphonic acid | 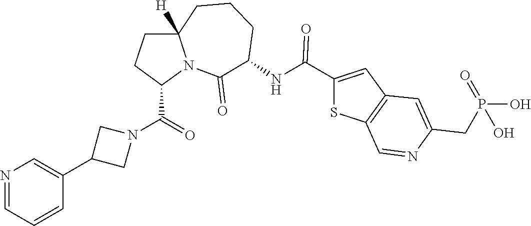 [SMILES: O=C(N[C@H]1CCC[C@H]2CC[C@@H](C(=O)N3CC(c4cccnc4)C3)N2C1=O)c1cc2cc(CP(=O)(O)O)ncc2s1] | 584.1 | ¹H NMR (400 MHz, CD₃OD) δ 9.40 (s, 1H), 8.83 (s, 1H), 8.74-8.50 (m, 2H), 8.26 (d, J = 6.0 Hz, 2H), 7.99-7.82 (m, 1H), 4.83-4.72 (m, 2H), 4.65-4.52 (m, 2H), 4.51-4.34 (m, 1H), 4.21-4.05 (m, 3H), 3.58-3.46 (m, 2H), 2.39-2.28 (m, 1H), 2.22-2.09 (m, 2H), 2.07-2.00 (m, |
| | | -N | | 2H), 2.00-1.95 (m, |
| | | N | | 1H), 1.95-1.80 (m, |
| | | | | 4H) |
| 575 | ((2-(((3S,6S,9aS)-3- (3-(4-ethylpyridin- 3-yl)azetidine-1- carbonyl)-5- oxooctahydro-1H- pyrrolo[1,2- a]azepin-6- yl)carbamoyl) imidazo[1,2-a] pyridin-6- yl)methyl) phosphonic acid | 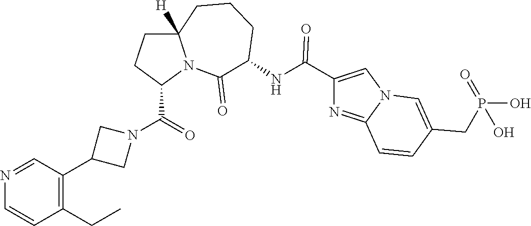 [SMILES: CCc1ccncc1C1CN(C(=O)[C@@H]2CC[C@@H]3CCC[C@H](NC(=O)c4cn5cc(CP(=O)(O)O)ccc5n4)C(=O)N32)C1] | 595.3 | ¹H NMR (400 MHz, CD₃OD) δ 8.88-9.13 (m, 1H), 8.64-8.72 (m, 1H), 8.40-8.60 (m, 2H), 7.87-7.99 (m, 1H), 7.60-7.78 (m, 2H), 4.66-4.85 (m, 3H), 4.58 (m, 1H), 4.45-4.54 (m, 1H), 4.35-4.44 (m, 1H), 4.04-4.21 (m, 2H), 3.13-3.25 (m, 2H), 2.90 (m, 2H), 2.28-2.38 (m, 1H), |
| | | | | 2.12-2.21 (m, 1H), |
| | | | | 1.91-2.10 (m, 5H), |
| | | | | 1.84 (s, 3H), 1.32 (m, |
| | | | | 3H |
| 576 | ((5-(((3S,6S,9aS)-3- (3-(4-ethylpyridin- 3-yl)azetidine-1- carbonyl)-5- oxooctahydro-1H- pyrrolo[1,2- a]azepin-6- yl)carbamoyl)benzo [b]thiophen-2- yl)methyl) phosphonic acid | 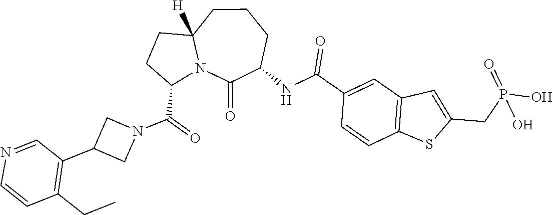 [SMILES: CCc1ccncc1C1CN(C(=O)[C@@H]2CC[C@@H]3CCC[C@H](NC(=O)c4ccc5sc(CP(=O)(O)O)cc5c4)C(=O)N32)C1] | 611.2 | 1H NMR (400 MHz, CD₃OD) δ 8.82-8.67 (m, 1H), 8.50 (t, J = 5.6 Hz, 1H), 8.23- 8.14 (m, 1H), 7.87- 7.79 (m, 1H), 7.78- 7.64 (m, 2H), 7.29 (s, 1H), 4.69-4.62 (m, 1H), 4.60-4.50 (m, 2H), 4.48-4.40 (m, 1H), 4.38-4.25 (m, 2H), 4.16-4.08 (m, 2H), 3.41-3.35 (m, |
| | | | | 2H), 2.83-2.72 (m, |
| | | | | 2H), 2.38-2.28 (m, |
| | | | | 1H), 2.22-2.12 (m, |
| | | | | 1H), 2.04-1.90 (m, |
| | | | | 5H), 1.84 (t, J = 13.2 |
| | | | | Hz, 3H), 1.41-1.34 |
| | | | | (m, 3H) |
| 577 | ((2-(((3S,6S,9aS)-3- (3-(6-fluoro-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 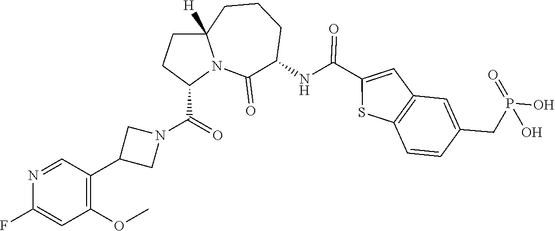 [SMILES: COc1cc(F)ncc1C1CN(C(=O)[C@@H]2CC[C@@H]3CCC[C@H](NC(=O)c4cc5cc(CP(=O)(O)O)ccc5s4)C(=O)N32)C1] | 630.9 | ¹H NMR (400 MHz, CD₃OD) δ 8.03-7.93 (m, 2H), 7.85 (s, 2H), 7.43 (d, J = 8.2 Hz, 1H), 6.73 (d, J = 15.6 Hz, 1H), 4.83-4.78 (m, 1H), 4.77-4.70 (m, 1H), 4.68-4.55 (m, 2H), 4.39 (t, J = 8.2 Hz, 1H), 4.31 (t, J = 9.2 Hz, 1H), 4.18- 4.10 (m, 2H), 3.97 (d, J = 1.2 Hz, 3H), 3.29- 3.21 (m, 2H), 2.34- |
| | | | | 2.27 (m, 1H), 1.98- |
| | | | | 1.83 (m, 9H) |
| 578 | ((2-(((3S,6S,9aS)-3- (3-(2-fluoro-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 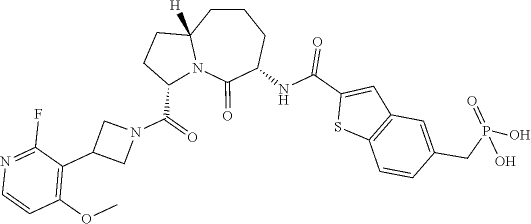 [SMILES: COc1ccnc(F)c1C1CN(C(=O)[C@@H]2CC[C@@H]3CCC[C@H](NC(=O)c4cc5cc(CP(=O)(O)O)ccc5s4)C(=O)N32)C1] | 630.9 | ¹H NMR (400 MHz, CDCl₃) δ 8.00-7.80 (m, 1H), 7.71-7.43 (m, 3H), 7.23-7.07 (m, 1H), 6.73-6.40 (m, 1H), 4.62 (d, J = 3.2 Hz, 2H), 4.49 (s, 2H), 4.42-4.25 (m, 4H), 4.16-4.09 (m, 1H), 3.93 (d, J = 14.4 Hz, 1H), 3.81 (s, 1H), 3.73 (s, 1H), 3.15- 3.01 (m, 1H), 2.22 (d, J = 7.6 Hz, 1H), 2.10- |
| | | | | 1.79 (m, 6H), 1.78- |
| | | | | 1.62 (m, 2H), 1.36- |
| | | | | 1.21 (m, 1H) |
| 579 | (fluoro(2- (((3S,6S,10aS)-5- oxo-3-(4-(pyridin-2- yl)piperidine-1- carbonyl)decahydro pyrrolo[1,2- a]azocin-6- yl)carbamoyl)benzo [b]thiophen-5- yl)methyl) phosphonic acid | 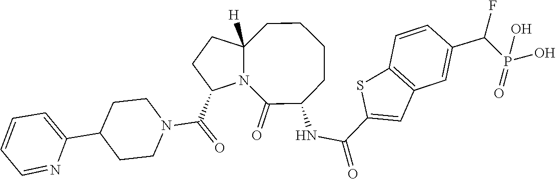 [SMILES: O=C(N[C@H]1CCCC[C@H]2CC[C@@H](C(=O)N3CCC(c4ccccn4)CC3)N2C1=O)c1cc2cc(C(F)P(=O)(O)O)ccc2s1] | 643.5 | ¹H NMR (400 MHz, CD₃OD) δ 8.72-8.27 (m, 2H), 8.08-8.02 (m, 1H), 8.01-7.66 (m, 4H), 7.60-7.51 (m, 1H), 5.92-5.65 (m, 1H), 5.09-4.92 (m, 2H), 4.67 (m, 1H), 4.50-4.26 (m, 2H), 3.30-3.23 (m, 2H), 2.81 (m, 1H), |
| | | | | 2.39-2.14 (m, 3H), |
| | | | | 2.12-1.91 (m, 8H), |
| | | | | 1.90-1.74 (m, 3H), |
| | | | | 1.73-1.61 (m, 2H) |
| 580 | ((2-(((3S,6S,9aS)-3- (3-(4-fluoro-2- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 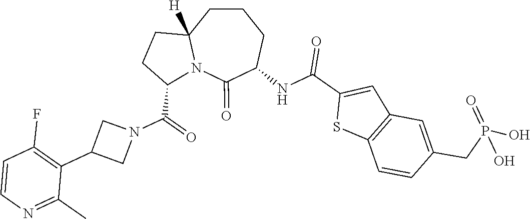 [SMILES: Cc1nccc(F)c1C1CN(C(=O)[C@@H]2CC[C@@H]3CCC[C@H](NC(=O)c4cc5cc(CP(=O)(O)O)ccc5s4)C(=O)N32)C1] | 615.1 | ¹H NMR (400 MHz, DMSO-d₆) δ 8.70 (t, J = 6.6 Hz, 1H), 8.37- 8.31 (m, 1H), 8.16 (d, J = 7.2 Hz, 1H), 7.90 (dd, J = 3.2, 8.4 Hz, 1H), 7.78 (s, 1H), 7.35 (d, J = 6.8 Hz, 1H), 7.21-7.13 (m, 1H), 4.77-4.62 (m, 2H), 4.57-4.50 (m, 1H), 4.48-4.45 (m, 1H), 4.39-4.25 (m, 2H), 4.23-4.15 (m, |
| | | | | 2H), 3.11 (s, 1H), |
| | | | | 3.06 (s, 2H), 2.47 (s, |
| | | | | 3H), 2.20 (d, J = 6.6 |
| | | | | Hz, 1H), 2.03-1.94 |
| | | | | (m, 2H), 1.84-1.66 |
| | | | | (m, 7H) |
| 581 | (fluoro(2- (((3S,6S,10aS)-3-(6- (5-fluoro-1-methyl- 2-oxo-1,2- dihydropyridin-4- yl)-5,7-dioxo-4,6- diazaspiro[2.4] heptane-4- carbonyl)-5- oxodecahydropyrrolo [1,2-a]azocin-6- yl)carbamoyl)benzo [b]thiophen-5- | 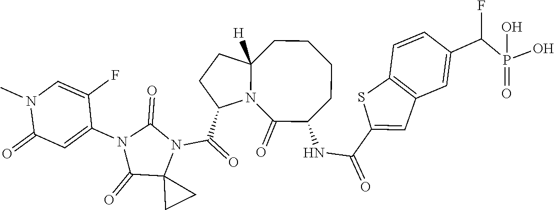 [SMILES: Cn1cc(F)c(N2C(=O)N(C(=O)[C@@H]3CC[C@@H]4CCCC[C@H](NC(=O)c5cc6cc(C(F)P(=O)(O)O)ccc6s5)C(=O)N43)C3(CC3)C2=O)cc1=O] | 731.9 | ¹H NMR (400 MHz, DMSO-d₆) δ 8.87 (d, J = 6.8 Hz, 1H), 8.28- 8.20 (m, 2H), 8.03 (d, J = 8.4 Hz, 1H), 7.96 (s, 1H), 7.53 (m, 1H), 6.68 (d, J = 6.8 Hz, 1H), 5.92-5.74 (m, 1H), 5.31 (m, 1H), 4.93 (m, 1H), 4.37-4.32 (m, 1H), 3.43 (s, 3H), 2.36 (m, 2H), 2.22-2.14 (m, |
| | yl)methyl) | | | 1H), 2.09 (m, 1H), |
| | phosphonic acid | | | 1.95-1.82 (m, 5H), |
| | | | | 1.80-1.69 (m, 3H), |
| | | | | 1.66 (s, 1H), 1.61- |
| | | | | 1.52 (m, 1H), 1.45- |
| | | | | 1.36 (m, 2H) |
| 582 | (fluoro(2- (((3S,6S,9aS)-3-(4- (5-fluoropyrimidin- 4-yl)piperidine-1- carbonyl)-5- oxooctahydro-1H- pyrrolo[1,2- a]azepin-6- yl)carbamoyl)benzo [b]thiophen-5- yl)methyl) phosphonic acid | 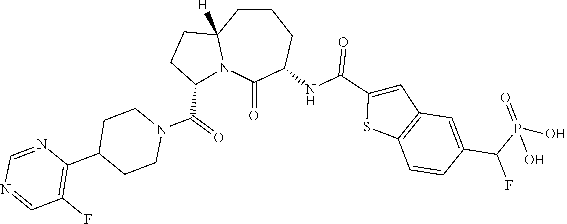 [SMILES: O=C(N[C@H]1CCC[C@H]2CC[C@@H](C(=O)N3CCC(c4ncncc4F)CC3)N2C1=O)c1cc2cc(C(F)P(=O)(O)O)ccc2s1] | 647.9 | ¹H NMR (400 MHz, CD₃OD) δ 8.93 (s, 1H), 8.76-8.53 (m, 1H), 8.05 (d, J = 6.0 Hz, 2H), 7.87 (d, J = 8.4 Hz, 1H), 7.64 (d, J = 8.0 Hz, 1H), 5.77- 5.54 (m, 1H), 5.18- 5.02 (m, 1H), 4.73 (d, J = 9.6 Hz, 1H), 4.61 (t, J = 11.2 Hz, 1H), 4.21 (t, J = 8.0 Hz, 1H), 4.12-4.03 (m, 1H), 3.49-3.35 (m, |
| | | | | 2H), 2.96-2.82 (m, |
| | | | | 1H), 2.38-2.27 (m, |
| | | | | 1H), 2.24-2.14 (m, |
| | | | | 1H), 2.06-2.01 (m, |
| | | | | 3H), 1.91 (d, J = 3.2 |
| | | | | Hz, 7H), 1.86-1.76 |
| | | | | (m, 2H) |
| 583 | (fluoro(2- (((3S,6S,9aS)-3-(3- (5-fluoropyrimidin- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 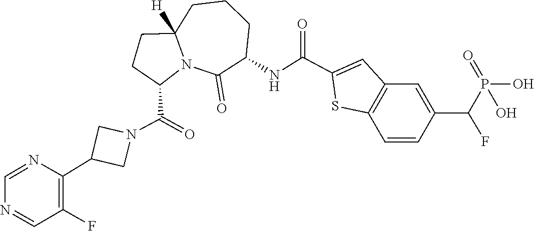 [SMILES: O=C(N[C@H]1CCC[C@H]2CC[C@@H](C(=O)N3CC(c4ncncc4F)C3)N2C1=O)c1cc2cc(C(F)P(=O)(O)O)ccc2s1] | 620.3 | ¹H NMR (400 MHz, DMSO-d₆) δ 9.09- 9.06 (m, 1H), 8.82- 8.75 (m, 2H), 8.24 (d, J = 12.4 Hz, 1H), 8.05- 7.96 (m, 2H), 7.53 (d, J = 8.4 Hz, 1H), 5.89-5.76 (m, 1H), 4.66-4.55 (m, 2H), 4.45 (d, J = 4.0 Hz, 1H), 4.30-4.14 (m, 3H), 4.07-3.98 (m, 2H), 2.22-2.16 (m, 1H), 2.02-1.93 (m, |
| | | | | 2H), 1.85-1.72 (m, |
| | | | | 7H) |
| 584 | ((2-(((3S,6S,9aS)-3- (3-(1-methyl-4-oxo- 1,4-dihydropyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 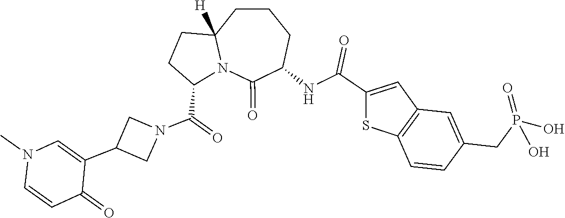 [SMILES: Cn1ccc(=O)c(C2CN(C(=O)[C@@H]3CC[C@@H]4CCC[C@H](NC(=O)c5cc6cc(CP(=O)(O)O)ccc6s5)C(=O)N43)C2)c1] | 613.2 | ¹H NMR (400 MHz, CD₃OD) δ 8.47-8.26 (m, 1H), 8.06-7.50 (m, 4H), 7.47-7.32 (m, 1H), 6.70-6.29 (m, 1H), 4.84-4.68 (m, 2H), 4.61-4.54 (m, 1H), 4.52-4.30 (m, 2H), 4.20 (s, 1H), 4.14-3.98 (m, 3H), 3.84 (d, J = 8.0 Hz, 2H), 3.28-3.18 (m, 2H), 2.39-2.25 (m, |
| | | | | 1H), 2.20-2.08 (m, |
| | | | | 2H), 2.08-1.90 (m, |
| | | | | 4H), 1.89-1.70 (m, |
| | | | | 3H) |
| 585 | ((2- (((3S,6S,9S,10aR)- 3-(3-(4- fluoropyridin-3- yl)azetidine-1- carbonyl)-9- methyl-5- oxodecahydropyrrolo [1,2-a]azocin-6- yl)carbamoyl)benzo [b]thiophen-5- yl)methyl) phosphonic acid | 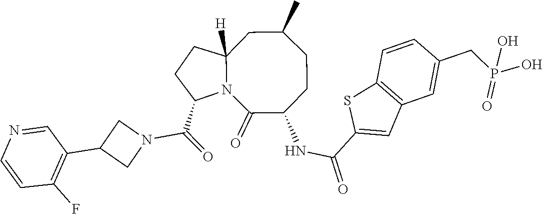 [SMILES: C[C@H]1CC[C@H](NC(=O)c2cc3cc(CP(=O)(O)O)ccc3s2)C(=O)N2[C@H](CC[C@H]2C(=O)N2CC(c3cnccc3F)C2)C1] | 629.1 | ¹H NMR (400 MHz, DMSO-d₆) δ 8.76- 8.53 (m, 3H), 8.18 (d, J = 10.4 Hz, 1H), 7.90 (m, 1H), 7.81-7.73 (m, 1H), 7.45-7.30 (m, 2H), 4.89-4.53 (m, 3H), 4.33 (d, J = 10.4 Hz, 2H), 4.24 (s, 1H), 4.12-4.06 (m, 2H), 3.13-3.05 (m, 2H), 2.27-2.18 (m, 1H), 2.03-1.81 (m, |
| | | | | 5H), 1.76-1.59 (m, |
| | | | | 4H), 1.31-1.20 (m, |
| | | | | 1H), 0.95 (d, J = 6.8 |
| | | | | Hz, 3H) |
| 586 | (fluoro(2- (((3S,6S,10aS)-3-(6- (1-isopropyl-2-oxo- 1,2-dihydropyridin- 4-yl)-5,7-dioxo-4,6- diazaspiro[2.4] heptane-4- carbonyl)-5- oxodecahydropyrrolo [1,2-a]azocin-6- yl)carbamoyl)benzo [b]thiophen-5- yl)methyl) | 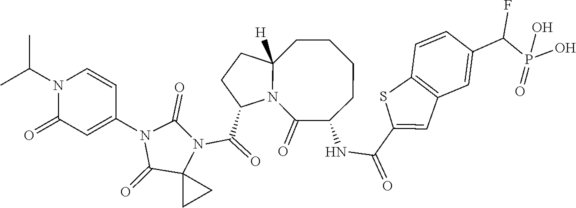 [SMILES: CC(C)n1ccc(N2C(=O)N(C(=O)[C@@H]3CC[C@@H]4CCCC[C@H](NC(=O)c5cc6cc(C(F)P(=O)(O)O)ccc6s5)C(=O)N43)C3(CC3)C2=O)cc1=O] | 742.1 | ¹H NMR (400 MHz, CD₃OD) δ 8.15-8.08 (m, 1H), 8.03-7.87 (m, 2H), 7.82 (d, J = 7.6 Hz, 1H), 7.60- 7.52 (m, 1H), 6.84- 6.62 (m, 2H), 5.90- 5.70 (m, 1H), 5.61- 5.50 (m, 1H), 5.20- 5.05 (m, 2H), 4.53- 4.42 (m, 1H), 2.63- 2.53 (m, 1H), 2.43- 2.23 (m, 3H), 2.10- |
| | phosphonic acid | | | 1.93 (m, 5H), 1.92- |
| | | | | 1.76 (m, 3H), 1.76- |
| | | | | 1.58 (m, 2H), 1.58- |
| | | | | 1.33 (m, 8H) |
| 587 | (fluoro(2- (((3S,6S,10aS)-3-(4- (4-fluoro-1H- pyrazol-3- yl)piperidine-1- carbonyl)-5- oxodecahydropyrrolo [1,2-a]azocin-6- yl)carbamoyl)benzo [b]thiophen-5- yl)methyl) phosphonic acid | 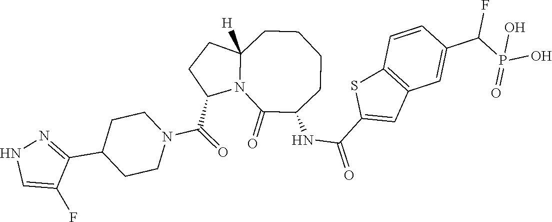 [SMILES: O=C(N[C@H]1CCCC[C@H]2CC[C@@H](C(=O)N3CCC(c4n[nH]cc4F)CC3)N2C1=O)c1cc2cc(C(F)P(=O)(O)O)ccc2s1] | 650.4 | ¹H NMR (400 MHz, CD₃OD) δ 8.10 (s, 1H), 8.01 (s, 1H), 7.93 (d, J = 8.8 Hz, 1H), 7.58 (d, J = 8.4 Hz, 1H), 7.48-7.39 (m, 1H), 5.91-5.75 (m, 1H), 5.08-5.02 (m, 1H), 4.99-4.91 (m, 3H), 4.53 (d, J = 13.2 Hz, 1H), 4.46- 4.42 (m, 1H), 4.20 (t, J = 12.8 Hz, 1H), 3.11- 3.02 (m, 1H), 2.84 |
| | | | | (t, J = 12.8 Hz, 1H), |
| | | | | 2.35-2.17 (m, 3H), |
| | | | | 2.06-1.94 (m, 7H), |
| | | | | 1.87-1.74 (m, 3H), |
| | | | | 1.67 (d, J = 12.0 Hz, |
| | | | | 2H) |
| 588 | (fluoro(2- (((3S,6S,10aS)-3-(4- (5-fluoropyrimidin- 4-yl)piperidine-1- carbonyl)-5- oxodecahydropyrrolo [1,2-a]azocin-6- yl)carbamoyl)benzo [b]thiophen-5- yl)methyl) phosphonic acid | 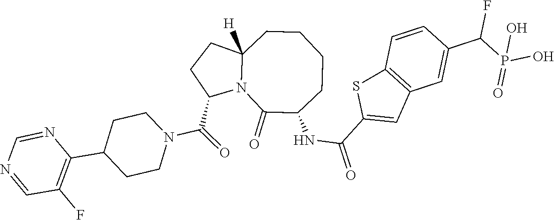 [SMILES: O=C(N[C@H]1CCCC[C@H]2CC[C@@H](C(=O)N3CCC(c4ncncc4F)CC3)N2C1=O)c1cc2cc(C(F)P(=O)(O)O)ccc2s1] | 662.0 | ¹H NMR (400 MHz, CD₃OD) 8.94-8.83 (m, 1H), 8.59 (dd, J = 1.8, 17.6 Hz, 1H), 8.10 (s, 1H), 8.01 (s, 1H), 7.93 (t, J = 7.6 Hz, 1H), 7.62-7.52 (m, 1H), 5.95-5.69 (m, 1H), 5.06 (d, J = 10.0 Hz, 1H), 5.00 (s, 1H), 4.67-4.54 (m, 1H), 4.51-4.38 (m, 1H), 4.28 (t, J = 13.2 Hz, 1H), 3.51-3.36 |
| | | | | (m, 2H), 2.94-2.83 |
| | | | | (m, 1H), 2.41-2.10 |
| | | | | (m, 4H), 2.09-1.95 |
| | | | | (m, 5H), 1.90 (s, 2H), |
| | | | | 1.86 (d, J = 4.8 Hz, |
| | | | | 3H), 1.68 (d, J = 10.8 |
| | | | | Hz, 2H) |
| 589 | ((2-(((3S,6S,10aS)- 3-(4-(1H-pyrazol-3- yl)piperidine-1- carbonyl)-5- oxodecahydropyrrolo [1,2-a]azocin-6- yl)carbamoyl)benzo [b]thiophen-5- yl)fluoromethyl) phosphonic acid | 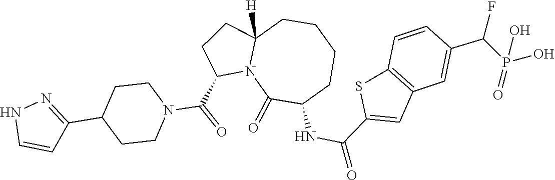 [SMILES: O=C(N[C@H]1CCCC[C@H]2CC[C@@H](C(=O)N3CCC(c4cc[nH]n4)CC3)N2C1=O)c1cc2cc(C(F)P(=O)(O)O)ccc2s1] | 632.3 | ¹H NMR (400 MHz, CD₃OD) δ 8.10 (s, 1H), 8.00 (s, 1H), 7.96-7.90 (m, 1H), 7.68-7.63 (m, 1H), 7.58 (d, J = 8.6 Hz, 1H), 6.34-6.25 (m, 1H), 5.90-5.72 (m, 1H), 5.09-4.99 (m, 1H), 4.99-4.90 (m, 3H), 4.59-4.50 (m, |
| | | | | 1H), 4.48-4.42 (m, |
| | | | | 1H), 4.27-4.14 (m, |
| | | | | 1H), 3.14-2.98 (m, |
| | | | | 1H), 2.90-2.77 (m, |
| | | | | 1H), 2.42-2.16 (m, |
| | | | | 3H), 2.12-1.91 (m, |
| | | | | 7H), 1.89-1.77 (m, |
| | | | | 2H), 1.73-1.56 (m, |
| | | | | 3H) |
| 590 | (fluoro(2- (((3S,6S,10aS)-5- oxo-3-(4- (pyrimidin-4- yl)piperidine-1- carbonyl)decahydro pyrrolo[ 1,2- a]azocin-6- yl)carbamoyl)benzo [b]thiophen-5- yl)methyl) phosphonic acid | 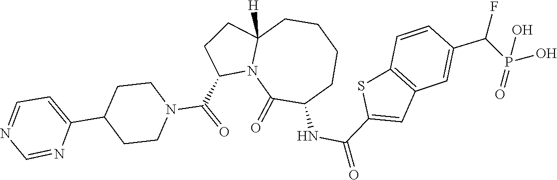 [SMILES: O=C(N[C@H]1CCCC[C@H]2CC[C@@H](C(=O)N3CCC(c4ccncn4)CC3)N2C1=O)c1cc2cc(C(F)P(=O)(O)O)ccc2s1] | 644.2 | ¹H NMR (400 MHz, CD₃OD) δ 9.04 (d, J = 19.2 Hz, 1H), 8.73- 8.56 (m, 1H), 8.10 (s, 1H), 8.01 (d, J = 7.2 Hz, 1H), 7.93 (t, J = 8.8 Hz, 1H), 7.63- 7.56 (m, 1H), 7.47 (d, J = 5.2 Hz, 1H), 5.92 -5.71 (m, 1H), 5.10- 5.03 (m, 2H), 4.70- 4.57 (m, 1H), 4.54- |
| | | | | 4.40 (m, 1H), 4.36- |
| | | | | 4.21 (m, 1H), 3.52- |
| | | | | 3.34 (m, 2H), 3.05 |
| | | | | (tdd, J = 4.0, 7.6, 11.6 |
| | | | | Hz, 1H), 2.92-2.76 |
| | | | | (m, 1H), 2.38-2.16 |
| | | | | (m, 3H), 2.11-1.92 |
| | | | | (m, 7H), 1.90-1.74 |
| | | | | (m, 3H), 1.73-1.61 |
| | | | | (m, 2H) |
| 591 | (fluoro(2- (((3S,6S,9aS)-3-(4- (4-fluoro-1H- pyrazol-3- yl)piperidine-1- carbonyl)-5- oxooctahydro-1H- pyrrolo[1,2- a]azepin-6- yl)carbamoyl)benzo [b]thiophen-5- yl)methyl) phosphonic acid | 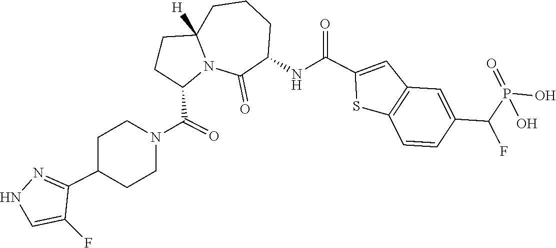 [SMILES: O=C(N[C@H]1CCC[C@H]2CC[C@@H](C(=O)N3CCC(c4n[nH]cc4F)CC3)N2C1=O)c1cc2cc(C(F)P(=O)(O)O)ccc2s1] | 636.4 | ¹H NMR (400 MHz, CD₃OD) δ 8.07 (s, 1H), 8.01 (s, 1H), 7.94 (d, J = 8.4 Hz, 1H), 7.58 (d, J = 8.4 Hz, 1H), 7.47-7.43 (m, 1H), 5.91-5.75 (m, 1H), 5.10-5.03 (m, 1H), 4.98-4.90 (m, 2H), 4.74 (d, J = 9.6 Hz, 1H), 4.55- 4.51 (m, 1H), 4.16- 4.02 (m, 2H), 3.10- 3.02 (m, 1H), 2.89- 2.80 (m, 1H), 2.34- |
| | | | | 2.29 (m, 1H), 2.24- |
| | | | | 2.14 (m, 1H), 2.09- |
| | | | | 1.90 (m, 8H), 1.84- |
| | | | | 1.73 (m, 3H) |
| 592 | ((2-(((3S,6S,9aS)-3- (3-(2,5- difluorophenyl) azetidine-1- carbonyl)-5- oxooctahydro-1H- pyrrolo[1,2- a]azepin-6- yl)carbamoyl)benzo [b]thiophen-5- yl)fluoromethyl) phosphonic acid | 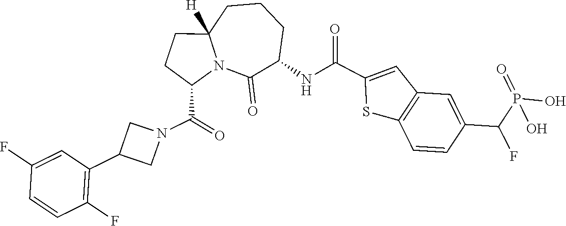 [SMILES: O=C(N[C@H]1CCC[C@H]2CC[C@@H](C(=O)N3CC(c4cc(F)ccc4F)C3)N2C1=O)c1cc2cc(C(F)P(=O)(O)O)ccc2s1] | 636.1 | ¹H NMR (400 MHz, CD₃OD) δ 8.13-8.01 (m, 2H), 7.97-7.94 (m, 1H), 7.59 (d, J = 8.1 Hz, 1H), 7.50- 7.20 (m, 1H), 7.19- 6.98 (m, 2H), 5.89 (d, J = 8.0 Hz, 1H), 4.93- 4.91 (m, 1H), 4.75 (d, J = 11.2 Hz, 1H), 4.70-4.54 (m, 2H), 4.42-4.32 (m, 1H), 4.25-4.12 (m, 1H), 4.10-4.05 (m, 2H), |
| | | | | 2.39-2.25 (m, 1H), |
| | | | | 2.20-2.11 (m, 1H), |
| | | | | 2.11-2.03 (m, 2H), |
| | | | | 2.03-1.98 (m, 1H), |
| | | | | 1.98-1.88 (m, 3H), |
| | | | | 1.88-1.78 (m, 2H) |
| 593 | ((2-(((3S,6S,9aS)-3- (3-(6- (difluoromethyl)-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 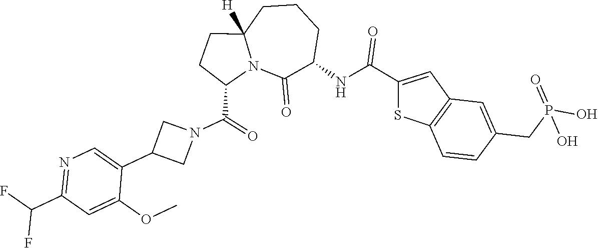 [SMILES: COc1cc(C(F)F)ncc1C1CN(C(=O)[C@@H]2CC[C@@H]3CCC[C@H](NC(=O)c4cc5cc(CP(=O)(O)O)ccc5s4)C(=O)N32)C1] | 663.1 | ¹H NMR (400 MHz, CD₃OD) δ 8.46-8.42 (m, 1H), 8.01-7.95 (m, 1H), 7.85-7.81 (m, 2H), 7.43-7.38 (m, 2H),6.89-6.60 (m, 1H), 4.73-4.65 (m, 3H), 4.59-4.33 (m, 2H), 4.14-4.12 (m, 2H), 4.04-4.03 (m, 3H), 3.76-3.56 (m, 1H), 3.31-3.27 (m, 2H), 2.30-2.29 (m, 1H), 2.09-2.06 (m, 2H), 1.98-1.82 (m, 7H) |
| 594 | ((2-(((3S,6S,9aS)-3- (3-(2- (difluoromethyl)-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 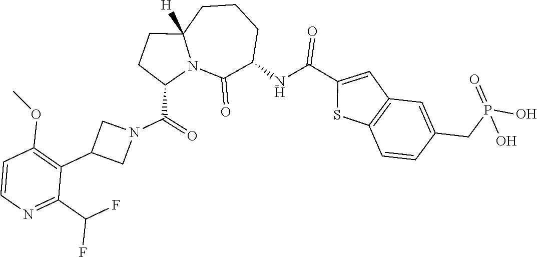 [SMILES: COc1ccnc(C(F)F)c1C1CN(C(=O)[C@@H]2CC[C@@H]3CCC[C@H](NC(=O)c4cc5cc(CP(=O)(O)O)ccc5s4)C(=O)N32)C1] | 663.3 | ¹H NMR (400 MHz, CD₃OD) δ 8.47-8.45 (m, 1H), 8.01-7.99 (m, 1H), 7.86-7.84 (m, 2H), 7.44-7.36 (m, 2H), 7.37-6.85 (m, 1 H), 4.75-4.73 (m, 2H), 4.58-4.52 (m, 2H), 4.51-4.22 (m, 3H), 4.17-4.08 (m, 4H), 3.28-3.23 (m, 2H), 2.34-2.33 (m, 1H), 1.98-1.85 (m, 9H) |
| 595 | (fluoro(2- (((3S,6S,9aS)-3-(3- (4-fluoro-1H- pyrazol-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 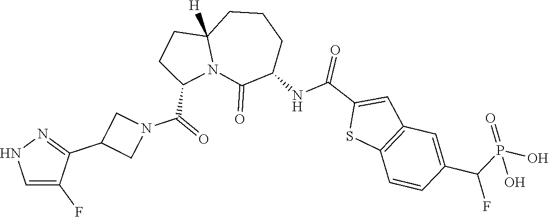 [SMILES: O=C(N[C@H]1CCC[C@H]2CC[C@@H](C(=O)N3CC(c4n[nH]cc4F)C3)N2C1=O)c1cc2cc(C(F)P(=O)(O)O)ccc2s1] | 608.5 | ¹H NMR (400 MHz, CD₃OD) δ 8.08 (d, J = 3.6 Hz, 1H), 8.03 (s, 1H), 7.97-7.94 (m, 1H), 7.60-7.50 (m, 2H), 5.90-5.76 (m, 1H), 4.75 (d, J = 10.8 Hz, 2H), 4.64 (d, J = 8.0 Hz, 1H), 4.59- 4.56 (m, 1H), 4.56- 4.33 (m, 2H), 4.14- 4.04 (m, 2H), 2.34- 2.27 (m, 1H), 2.17- |
| | | | | 2.06 (m, 2H), 2.00- |
| | | | | 1.83 (m, 7H) |
| 596 | (fluoro(2- (((3S,6S,9aS)-3-(3- (5-fluoropyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 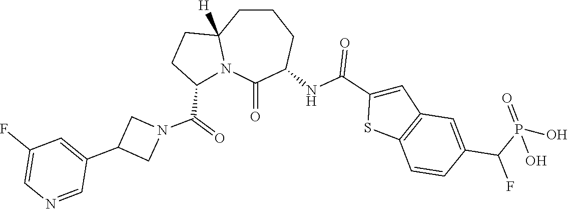 [SMILES: O=C(N[C@H]1CCC[C@H]2CC[C@@H](C(=O)N3CC(c4cncc(F)c4)C3)N2C1=O)c1cc2cc(C(F)P(=O)(O)O)ccc2s1] | 619.0 | ¹H NMR (400 MHz, DMSO-d₆) δ 8.82- 8.76 (m, 1H), 8.50- 8.42 (m, 2H), 8.25 (d, J = 11.6 Hz, 1H), 8.07- 7.96 (m, 2H), 7.88 (d, J = 9.8 Hz, 1H), 7.53 (d, J = 8.6 Hz, 1H), 5.92-5.75 (m, 1H), 4.80-4.60 (m, 2H), 4.47-4.38 (m, 2H), 4.31-4.22 (m, 2H), 4.00 (s, 1H), |
| | | | | 3.90 (s, 1H), 2.22- |
| | | | | 2.16 (m, 1H), 2.08- |
| | | | | 1.93 (m, 2H), 1.90- |
| | | | | 1.80 (m, 3H), 1.79- |
| | | | | 1.59 (m, 4H) |
| 597 | ((2-(((3S,6S,9aS)-3- (3-(4-ethoxypyridin- 3-yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 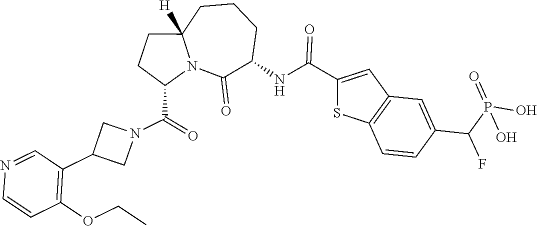 [SMILES: CCOc1ccncc1C1CN(C(=O)[C@@H]2CC[C@@H]3CCC[C@H](NC(=O)c4cc5cc(C(F)P(=O)(O)O)ccc5s4)C(=O)N32)C1] | 645.3 | ¹H NMR (400 MHz, CD₃OD) δ 8.36-8.21 (m, 2H), 8.00-7.88 (m, 1H), 7.84 (t, J = 9.2 Hz, 2H), 7.69- 7.49 (m, 1H), 7.38- 7.24 (m, 1H), 5.87- 5.61 (m, 1H), 4.81 (d, J = 2.8 Hz, 1H), 4.74 (d, J = 11.6 Hz, 1H), 4.68-4.57 (m, 1H), 4.54-4.47 (m, 1H), 4.45-4.36 (m, 1H), 4.35-4.29 (m, 2H), |
| | | | | 4.21-3.97 (m, 3H), |
| | | | | 2.39-2.25 (m, 1H), |
| | | | | 2.20-2.06 (m, 2H), |
| | | | | 2.03-1.78 (m, 7H), |
| | | | | 1.55-1.45 (m, 3H) |
| 598 | (fluoro(2- (((3S,6S,9aS)-3-(3- (4-(oxetan-3- yloxy)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 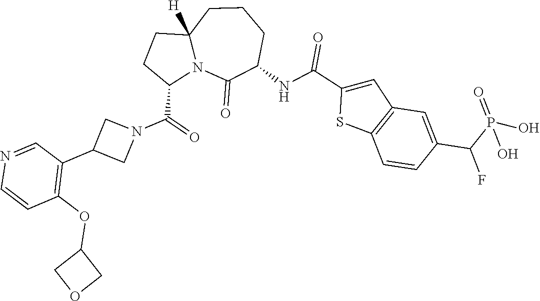 [SMILES: O=C(N[C@H]1CCC[C@H]2CC[C@@H](C(=O)N3CC(c4cnccc4OC4COC4)C3)N2C1=O)c1cc2cc(C(F)P(=O)(O)O)ccc2s1] | 673.4 | ¹H NMR (400 MHz, DMSO-d₆) δ 8.81- 8.58 (m, 3H), 8.29- 8.14 (m, 1H), 8.07- 7.90 (m, 2H), 7.52 (d, J = 7.6 Hz, 1H), 7.21- 7.07 (m, 1H), 5.91- 5.74 (m, 1H), 5.66- 5.55 (m, 1H), 5.04- 4.92 (m, 2H), 4.77 (t, J = 9.6 Hz, 1H), 4.68- 4.59 (m, 3H), 4.49- 4.36 (m, 2H), 4.31- 4.09 (m, 3H), 3.99 (s, 1H), 2.27-2.11 (m, 1H), 2.08-1.93 (m, 2H), 1.90-1.75 (m, 4H), 1.74-1.51 (m, 3H) |
| 599 | ((S)-fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 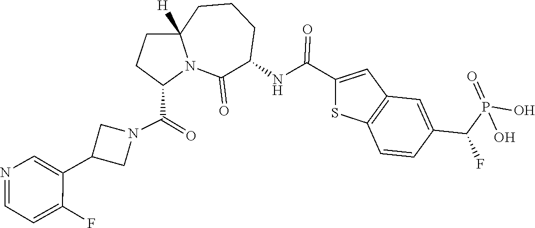 [SMILES: O=C(N[C@H]1CCC[C@H]2CC[C@@H](C(=O)N3CC(c4cnccc4F)C3)N2C1=O)c1cc2cc([C@@H](F)P(=O)(O)O)ccc2s1] | 619.1 | ¹H NMR (400 MHz, DMSO-d₆) δ 8.74 (t, J = 7.2 Hz, 1H), 8.68 (dd, J = 2.0, 10.4 Hz, 1H), 8.57-8.49 (m, 1H), 8.20 (d, J = 10.0 Hz, 1H), 7.93-7.85 (m, 2H), 7.51 (d, J = 8.4 Hz, 1H), 7.36- 7.28 (m, 1H), 5.54- 5.35 (m, 1H), 4.82- 4.57 (m, 2H), 4.54- 4.35 (m, 2H), 4.34- 4.22 (m, 1H), 4.16- |
| | | | | 4.02 (m, 2H), 4.00 (d, |
| | | | | J = 6.0 Hz, 1H), 2.24- |
| | | | | 2.15 (m, 1H), 2.09- |
| | | | | 1.92 (m, 2H), 1.90- |
| | | | | 1.75 (m, 4H), 1.75- |
| | | | | 1.60 (m, 3H) |
| 600 | ((R)-fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 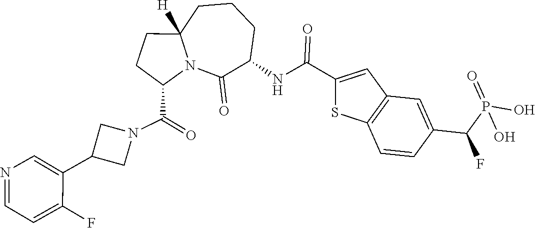 [SMILES: O=C(N[C@H]1CCC[C@H]2CC[C@@H](C(=O)N3CC(c4cnccc4F)C3)N2C1=O)c1cc2cc([C@H](F)P(=O)(O)O)ccc2s1] | 619.1 | ¹H NMR (400 MHz, D₂O) δ 8.85-8.65 (m, 1H), 8.59 (t, J = 5.6 Hz, 1H), 7.95- 7.83 (m, 3H), 7.74- 7.63 (m, 1H), 7.56- 7.49 (m, 1H), 5.85- 5.67 (m, 1H), 4.72- 4.68 (m, 1H), 4.52 (d, J = 16.0 Hz, 1H), 4.49- 4.39 (m, 2H), 4.36- 4.20 (m, 2H), 4.20- 4.10 (m, 1H), 4.09- 4.02 (m, 1H), 2.32- |
| | | | | 2.22 (m, 1H), 2.17- |
| | | | | 2.09 (m, 1H), 2.07- |
| | | | | 2.02 (m, 1H), 1.98- |
| | | | | 1.89 (m, 2H), 1.88- |
| | | | | 1.74 (m, 4H), 1.72- |
| | | | | 1.65 (m, 1H) |
| 601 | ((2-(((3S,6S,10aS)- 3-(6-(2,2- difluorobenzo[d][1, 3]dioxol-4-yl)-5,7- dioxo-4,6- diazaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 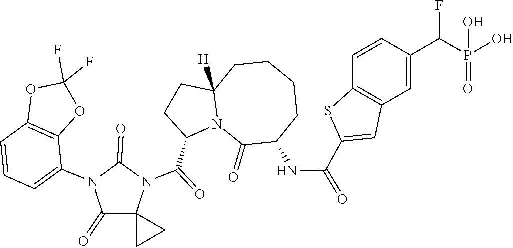 [SMILES: O=C(N[C@H]1CCCC[C@H]2CC[C@@H](C(=O)N3C(=O)N(c4cccc5c4OC(F)(F)O5)C(=O)C34CC4)N2C1=O)c1cc2cc(C(F)P(=O)(O)O)ccc2s1] | 763.1 | ¹H NMR (400 MHz, CD₃OD) δ 8.11 (s, 1H), 8.03 (s, 1H), 7.95 (d, J = 8.4 Hz, 1H), 7.60 (d, J = 8.0 Hz, 1H), 7.40-7.23 (m, 3H), 5.92-5.70 (m, 1H), 5.57 (dd, J = 8.0, 9.2 Hz, 1H), 5.05 (dd, J = 4.8, 11.2 Hz, 1H), 4.49 (dd, J = 8.8, 10.2 Hz, 1H), 2.66- 2.53 (m, 1H), 2.52- 2.41 (m, 1H), 2.40- 2.26 (m, 2H), 2.15- |
| | | | | 1.93 (m, 6H), 1.93- |
| | | | | 1.78 (m, 3H), 1.77- |
| | | | | 1.60 (m, 2H), 1.55- |
| | | | | 1.49 (m, 1H) |
| 602 | (fluoro(2- (((3S,6S,9aS)-3-(2- methyl-3-(pyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 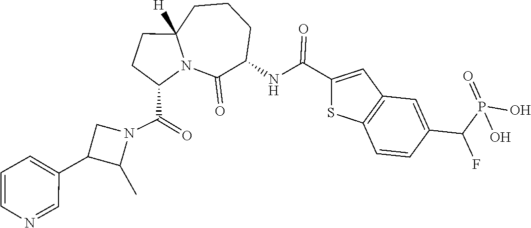 [SMILES: CC1C(c2cccnc2)CN1C(=O)[C@@H]1CC[C@@H]2CCC[C@H](NC(=O)c3cc4cc(C(F)P(=O)(O)O)ccc4s3)C(=O)N21] | 615.1 | ¹H NMR (400 MHz, CD₃OD) δ 8.77-8.61 (m, 2H), 8.60-8.44 (m, 1H), 8.12-7.97 (m, 2H), 7.91 (d, J = 8.0 Hz, 1H), 7.80- 7.68 (m, 1H), 7.66- 7.55 (m, 1H), 5.91- 5.64 (m, 1H), 4.79- 4.72 (m, 1H), 4.66 (t, J = 8.8 Hz, 1H), 4.60- 4.35 (m, 3H), 4.17- 4.05 (m, 1H), 3.64- 3.59 (m, 1H), 2.42- |
| | | | | 2.25 (m, 1H), 2.23- |
| | | | | 2.04 (m, 3H), 2.01- |
| | | | | 1.76 (m, 6H), 1.69- |
| | | | | 1.55 (m, 3H) |
| 603 | ((2- (((3S,6S,9R,10aR)- 3-(3-(4- fluoropyridin-3- yl)azetidine-1- carbonyl)-9- methyl-5- oxodecahydropyrrolo [1,2-a]azocin-6- yl)carbamoyl)benzo [b]thiophen-5- yl)methyl) phosphonic acid | 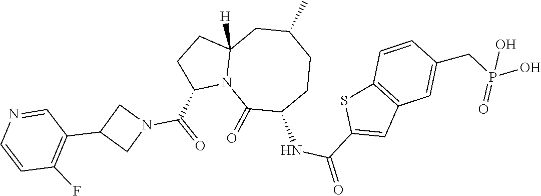 [SMILES: C[C@@H]1CC[C@H](NC(=O)c2cc3cc(CP(=O)(O)O)ccc3s2)C(=O)N2[C@H](CC[C@H]2C(=O)N2CC(c3cnccc3F)C2)C1] | 629.4 | ¹H NMR (400 MHz, DMSO-d₆) δ 8.83- 8.67 (m, 2H), 8.61- 8.54 (m, 1H), 8.20 (d, J = 8.8 Hz, 1H), 7.92- 7.89 (m, 1H), 7.78 (s, 1H), 7.45-7.32 (m, 2H), 4.97-4.80 (m, 2H), 4.63-4.56 (m, 1H), 4.36-4.30 (m, 3H), 4.12 (d, J = 7.6 Hz, 2H), 3.13- 3.04 (m, 2H), 2.22- |
| | | | | 2.13 (m, 1H), 2.10- |
| | | | | 1.97 (m, 2H), 1.88- |
| | | | | 1.64 (m, 6H), 1.51 (d, |
| | | | | J = 12.0 Hz, 1H), 1.32- |
| | | | | 1.24 (m, 1H), 0.95 |
| | | | | (d, J = 6.0 Hz, 3H) |
| 604 | (fluoro(2- (((3S,6S,9aS)-5- oxo-3-(4- (pyrimidin-4- yl)piperidine-1- carbonyl)octahydro- 1H-pyrrolo[1,2- a]azepin-6- yl)carbamoyl)benzo [b]thiophen-5- yl)methyl) phosphonic acid | 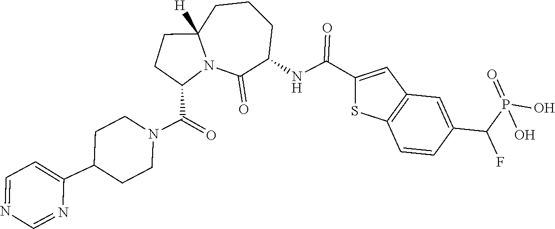 [SMILES: O=C(N[C@H]1CCC[C@H]2CC[C@@H](C(=O)N3CCC(c4ccncn4)CC3)N2C1=O)c1cc2cc(C(F)P(=O)(O)O)ccc2s1] | 630.2 | ¹H NMR (400 MHz, CD₃OD) δ 9.07 (d, J = 8.4 Hz, 1H), 8.67 (dd, J = 5.6, 17.2 Hz, 1H), 8.11-7.90 (m, 3H), 7.64-7.47 (m, 2H), 5.94-5.73 (m, 1H), 5.08 (m, 1H), 4.74 ( d, J = 9.6 Hz, 1H), 4.67-4.56 (m, 1H), 4.20 ( t, J = 13.6 Hz, 1H), 4.08 ( d, J = 5.6 Hz, 1H), 3.39- 3.33 (m, 1H), 3.16- |
| | | | | 3.00 (m, 1H), 2.85 |
| | | | | (m, 1H), 2.37-2.16 |
| | | | | (m, 2H), 2.09 ( d, J = |
| | | | | 5.2 Hz, 1H), 2.06- |
| | | | | 1.97 (m, 4H), 1.96- |
| | | | | 1.90 (m, 3H), 1.89- |
| | | | | 1.80 (m, 3H), 1.80- |
| | | | | 1.69 (m, 1H) |
| 605 | ((2-(((3S,6S,9aS)-3- (3-(4- hydroxypyrimidin- 5-yl)azetidine-1- carbonyl)-5- oxooctahydro-1H- pyrrolo[1,2- a]azepin-6- yl)carbamoyl)benzo [b]thiophen-5- yl)methyl) phosphonic acid | 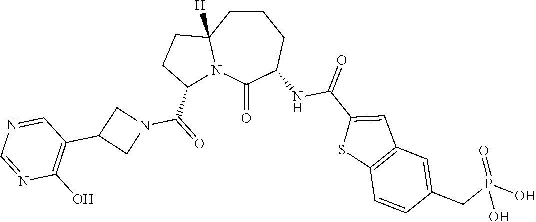 [SMILES: O=C(N[C@H]1CCC[C@H]2CC[C@@H](C(=O)N3CC(c4cncnc4O)C3)N2C1=O)c1cc2cc(CP(=O)(O)O)ccc2s1] | 600.1 | ¹H NMR (400 MHz, DMSO-d₆) δ 8.70 (d, J = 7.6 Hz, 1H), 8.16 (m, 2H), 7.95 (s, 1H), 7.91 (m, 1H), 7.79 (s, 1H), 7.40-7.32 (m, 1H), 4.69-4.54 (m, 2H), 4.43 (m, 1H), 4.35-4.28 (m, 1H), 4.10 (d, J = 6.8 Hz, 1H), 3.98-3.92 (m, 2H), 3.79-3.75 (m, 1H), 3.15-3.06 (m, 2H), 2.26-2.13 (m, |
| | | | | 1H), 2.09-1.92 (m, |
| | | | | 2H), 1.91-1.58 (m, |
| | | | | 7H |
| 606 | ((2-(((3S,6S,9aS)-3- (3-(4- (methylamino) pyridin-3-yl) azetidine-1- carbonyl)-5- oxooctahydro-1H- pyrrolo[1,2- a]azepin-6- yl)carbamoyl)benzo [b]thiophen-5- yl)methyl) phosphonic acid | 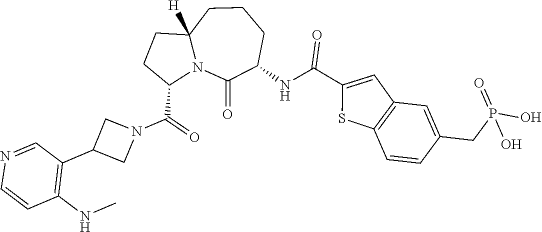 [SMILES: CNc1ccncc1C1CN(C(=O)[C@@H]2CC[C@@H]3CCC[C@H](NC(=O)c4cc5cc(CP(=O)(O)O)ccc5s4)C(=O)N32)C1] | 612.4 | ¹H NMR (400 MHz, CD₃OD) δ 8.22-8.09 (m, 2H), 8.03-7.94 (m, 1H), 7.88-7.82 (m, 2H), 7.45-7.41 (m, 1H), 6.95-6.89 (m, 1H), 4.75-4.67 (m, 2H), 4.63-4.56 (m, 1H), 4.55-4.51 (m, 1H), 4.45-4.38 (m, 1H), 4.11-4.06 (m, 1H), 4.05-3.99 (m, 1H), 3.97-3.90 (m, 1H), 3.28 (d, J = |
| | | | | 3.2 Hz, 1H), 3.22 (d, |
| | | | | J = 3.2 Hz, 1H), 3.03 |
| | | | | (s, 3H), 2.36-2.27 |
| | | | | (m, 1H), 2.18-2.07 |
| | | | | (m, 2H), 2.02-1.79 |
| | | | | (m, 7H) |
| 607 | (fluoro(2- (((3S,6S,9aS)-3-(3- (5-hydroxy-1- methyl-1H-pyrazol- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 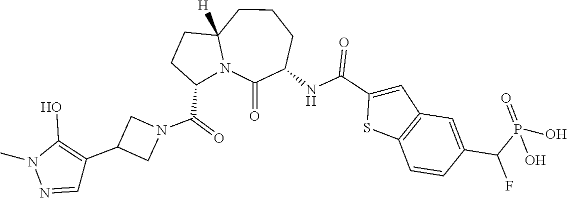 [SMILES: Cn1ncc(C2CN(C(=O)[C@@H]3CC[C@@H]4CCC[C@H](NC(=O)c5cc6cc(C(F)P(=O)(O)O)ccc6s5)C(=O)N43)C2)c1O] | 620.0 | ¹H NMR (400 MHz, CD₃OD) δ 8.09 (d, J = 4.4 Hz, 1H), 8.04 (s, 1H), 7.96 (d, J = 3.6, 8.8 Hz, 1H), 7.81- 7.64 (m, 1H), 7.61 (d, J = 8.4 Hz, 1H), 5.94-5.74 (m, 1H), 4.78-4.74 (m, 1H), 4.62-4.50 (m, 2H), 4.47-4.35 (m, 1H), 4.34-4.25 (m, 1H), 4.08 (s, 1H), 4.03- |
| | | | | 3.92 (m, 1H), 3.79- |
| | | | | 3.70 (m, 1H), 3.53 (d, |
| | | | | J = 10.0 Hz, 3H), 2.36- |
| | | | | 2.27 (m, 1H), 2.18- |
| | | | | 2.07 (m, 2H), 2.07- |
| | | | | 1.98 (m, 2H), 1.98- |
| | | | | 1.92 (m, 2H), 1.92- |
| | | | | 1.81 (m, 3H) |
| 608 | (fluoro(2- (((3S,6S,9aS)-3-(3- (2-hydroxyphenyl) azetidine-1- carbonyl)-5- oxooctahydro-1H- pyrrolo[1,2- a]azepin-6- yl)carbamoyl)benzo [b]thiophen-5- yl)methyl) phosphonic acid | 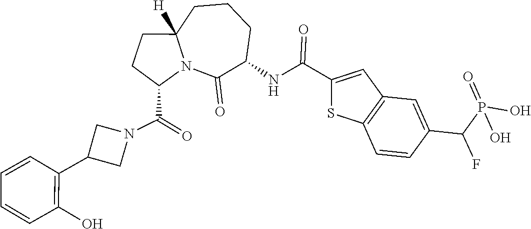 [SMILES: O=C(N[C@H]1CCC[C@H]2CC[C@@H](C(=O)N3CC(c4ccccc4O)C3)N2C1=O)c1cc2cc(C(F)P(=O)(O)O)ccc2s1] | 616.1 | ¹H NMR (400 MHz, DMSO-d₆) δ 8.73- 8.70 (m, 1H), 8.18 (d, J = 17.6 Hz, 1H), 7.93- 7.83 (m, 2H), 7.55- 7.45 (m, 1H), 7.23- 7.29 (m, 1H), 7.07- 7.00 (m, 1H), 6.88- 6.70 (m, 2H), 5.54- 5.34 (m, 1H), 4.74- 4.42 (m, 3H), 4.40- 4.11 (m, 2H), 4.01- 3.91 (m, 3H), 2.24- 2.13 (m, 1H), 2.07- |
| | | | | 1.91 (m, 2H), 1.89- |
| | | | | 1.77 (m, 3H), 1.75- |
| | | | | 1.63 (m, 4H) |
| 609 | (fluoro(2- (((3S,6S,9aS)-5- oxo-3-(3-(6- oxopyrimidin- 1(6H)-yl)azetidine-1- carbonyl)octahydro- 1H-pyrrolo[1,2- a]azepin-6- yl)carbamoyl)benzo [b]thiophen-5- yl)methyl) phosphonic acid | 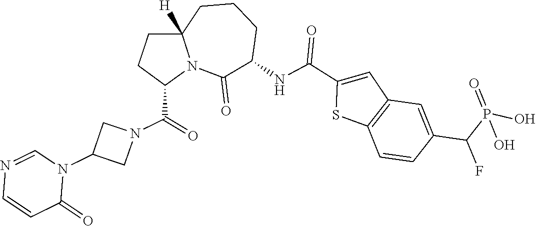 [SMILES: O=C(N[C@H]1CCC[C@H]2CC[C@@H](C(=O)N3CC(n4cnccc4=O)C3)N2C1=O)c1cc2cc(C(F)P(=O)(O)O)ccc2s1] | 617.8 | ¹H NMR (400 MHz, CD₃OD) δ 8.74-8.40 (m, 1H), 8.10 (d, J = 6.4 Hz, 1H), 8.03 (s, 1H), 7.97-7.94 (m, 2H), 7.59 (d, J = 8.4 Hz, 1H), 6.54-6.44 (m, 1H), 5.93-5.73 (m, 1H), 5.44-5.12 (m, 1H), 4.82 (d, J = 3.6 Hz, 1H), 4.77- 4.70 (m, 2H), 4.58- 4.33 (m, 3H), 4.07 (d, J = 6.8 Hz, 1H), 2.37- |
| | | | | 2.25 (m, 1H), 2.20- |
| | | | | 1.99 (m, 4H), 1.98- |
| | | | | 1.75 (m, 5H) |
| 610 | (fluoro(2- (((3S,6S,9aS)-3-(3- (3-fluoropyridin-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 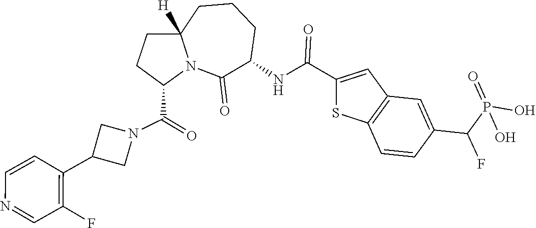 [SMILES: O=C(N[C@H]1CCC[C@H]2CC[C@@H](C(=O)N3CC(c4ccncc4F)C3)N2C1=O)c1cc2cc(C(F)P(=O)(O)O)ccc2s1] | 618.9 | ¹H NMR (400 MHz, DMSO-d₆) δ 8.75 (d, J = 8.0 Hz, 1H), 8.52 (d, J = 10.8 Hz, 1H), 8.43-8.38 (m, 1H), 8.25-8.23 (m, 1H), 8.04-7.97 (m, 2H), 7.59-7.48 (m, 2H), 5.89-5.76 (m, 1H), 4.65-4.62 (m, 1H), 4.43 (d, J = 7.2 Hz, 1H), 4.32-4.25 (m, 2H), 4.12 (d, J = 7.6 Hz, 1H), 3.98 (d, J = |
| | | | | 6.4 Hz, 3H), 2.19 (d, |
| | | | | J = 3.6 Hz, 1H), 1.97- |
| | | | | 1.94 (m, 2H), 1.85- |
| | | | | 1.80 (m, 3H), 1.77- |
| | | | | 1.72 (m, 4H) |
| 611 | (fluoro(2- (((3S,6S,9aS)-5- oxo-3-(3-(pyridin-4- yl)azetidine-1- carbonyl)octahydro- 1H-pyrrolo[1,2- a]azepin-6- yl)carbamoyl)benzo [b]thiophen-5- yl)methyl) phosphonic acid | 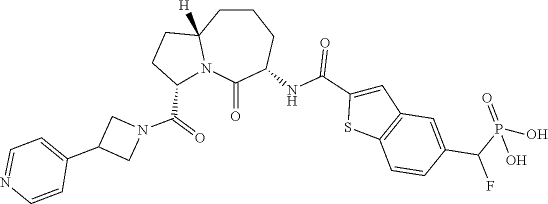 [SMILES: O=C(N[C@H]1CCC[C@H]2CC[C@@H](C(=O)N3CC(c4ccncc4)C3)N2C1=O)c1cc2cc(C(F)P(=O)(O)O)ccc2s1] | 601.4 | ¹H NMR (400 MHz, DMSO-d₆) δ 8.80- 8.67 (m, 1H), 8.56- 8.53 (m, 1H), 8.51- 8.46 (m, 1H), 8.20 (d, J = 3.2 Hz, 1H), 7.90 (d, J = 8.0 Hz, 2H), 7.52 (t, J = 7.2 Hz, 1H), 7.39 (t, J = 13.2 Hz, 2H), 5.55-5.34 (m, 1H), 4.82-4.59 (m, 2H), 4.44-4.38 (m, 1H), 4.30-4.20 |
| | | | | (m, 2H), 3.99 (s, 1H), |
| | | | | 3.91-3.85 (m, 2H), |
| | | | | 2.22-2.12 (m, 1H), |
| | | | | 2.03-1.93 (m, 2H), |
| | | | | 1.85-1.81 (m, 4H), |
| | | | | 1.74-1.69 (m, 2H), |
| | | | | 1.64 (d, J = 9.6 Hz, |
| | | | | 1H) |
| 612 | (fluoro(2- (((3S,6S,9aS)-3-(3- (4- isopropoxypyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 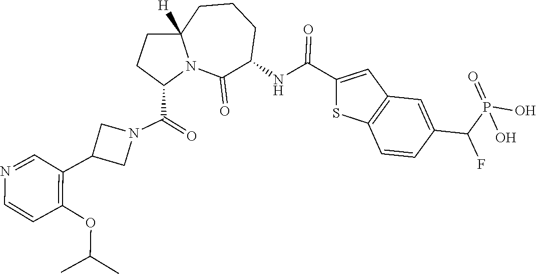 [SMILES: CC(C)Oc1ccncc1C1CN(C(=O)[C@@H]2CC[C@@H]3CCC[C@H](NC(=O)c4cc5cc(C(F)P(=O)(O)O)ccc5s4)C(=O)N32)C1] | 659.3 | ¹H NMR (400 MHz, CD₃OD) δ 8.35-8.17 (m, 2H), 8.07-7.76 (m, 3H), 7.66-7.50 (m, 1H), 7.42-7.25 (m, 1H), 5.90-5.65 (m, 1H), 5.08-5.00 (m, 1H), 4.84-4.72 (m, 2H), 4.64-4.48 (m, 2H), 4.35-4.27 (m, 1H), 4.16-3.88 (m, 3H), 2.38-2.25 (m, 1H), 2.20-2.06 (m, 2H), 2.05-1.78 (m, 7H), 1.53-1.37 (m, 6H) |
| 613 | ((2-(((3S,6S,9aS)-3- (3-(1H-pyrazol-1- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 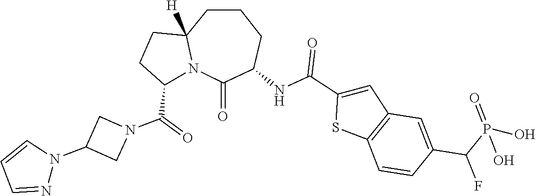 [SMILES: O=C(N[C@H]1CCC[C@H]2CC[C@@H](C(=O)N3CC(n4cccn4)C3)N2C1=O)c1cc2cc(C(F)P(=O)(O)O)ccc2s1] | 590.3 | ¹H NMR (400 MHz, CD³OD) 8.15-7.98 (m, 2H), 7.93 (d, J = 2.4 Hz, 2H), 7.69- 7.50 (m, 2H), 6.37- 6.30 (m, 1H), 5.73- 5.56 (m, 1H), 5.38- 5.25 (m, 1H), 4.97- 4.93 (m, 1H), 4.74 (d, J = 11.2 Hz, 1H), 4.63- 4.53 (m, 2H), 4.53- 4.40 (m, 1H), 4.38- |
| | | | | 4.24 (m, 1H), 4.07 (d, |
| | | | | J = 6.1 Hz, 1H), 2.19- |
| | | | | 2.07 (m, 2H), 2.07- |
| | | | | 2.00 (m, 2H), 1.99- |
| | | | | 1.90 (m, 2H), 1.90- |
| | | | | 1.76 (m, 4H) |
| 614 | (fluoro(2- (((3S,6S,9aS)-3-(3- (4-fluoro-1H- pyrazol-1- yl)azetidine-1- carbonyl)-5- oxooctahydro-1H- pyrrolo[1,2- a]azepin-6- yl)carbamoyl)benzo [b]thiophen-5- yl)methyl) phosphonic acid | 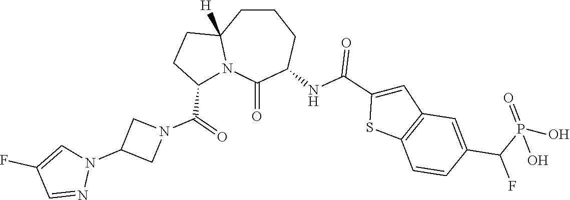 [SMILES: O=C(N[C@H]1CCC[C@H]2CC[C@@H](C(=O)N3CC(n4cc(F)cn4)C3)N2C1=O)c1cc2cc(C(F)P(=O)(O)O)ccc2s1] | 608.1 | ¹H NMR (400 MHz, CD₃OD) δ 8.08 (s, 1H), 8.03 (s, 1H), 7.98-7.93 (m, 1H), 7.85-7.67 (m, 1H), 7.62-7.54 (m, 1H), 7.54-7.42 (m, 1H), 5.91-5.75 (m, 1H), 5.22-5.14 (m, 1H), 4.94-4.90 (m, 1H), 4.83-4.67 (m, 2H), 4.55 (m, 1H), 4.51- 4.38 (m, 1H), 4.35- |
| | | | | 4.23 (m, 1H), 4.11- |
| | | | | 4.02 (m, 1H), 2.36- |
| | | | | 2.24 (m, 1H), 2.08 |
| | | | | (m, 2H), 2.05-1.99 |
| | | | | (m, 2H), 1.98-1.90 |
| | | | | (m, 2H), 1.90-1.79 |
| | | | | (m, 3H) |
| 615 | (fluoro(2- (((3S,6S,10aS)-3-(3- (2-methoxyethoxy)- 3-(pyridin-2- yl)azetidine-1- carbonyl)-5- oxodecahydropyrrol o[1,2-a]azocin-6- yl)carbamoyl)benzo [b]thiophen-5- yl)methyl) phosphonic acid | 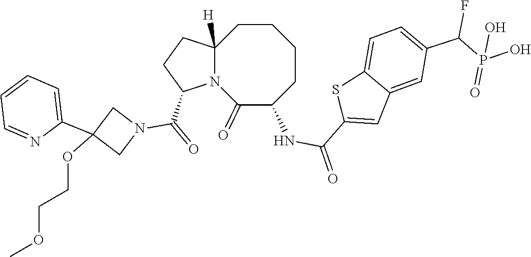 [SMILES: COCCOC1(c2ccccn2)CN(C(=O)[C@@H]2CC[C@@H]3CCCC[C@H](NC(=O)c4cc5cc(C(F)P(=O)(O)O)ccc5s4)C(=O)N32)C1] | 689.1 | ¹H NMR (400 MHz, D₂O) δ 8.73-8.55 (m, 1H), 8.34-8.21 (m, 1H), 8.20-8.03 (m, 1H), 8.02-7.81 (m, 1H), 7.78 (d, J = 8.4 Hz, 1H), 7.69- 7.60 (m, 2H), 7.51- 7.38 (m, 1H), 5.81- 5.62 (m, 1H), 4.97- 4.93 (m, 1H), 4.80- 4.79 (m, 2H), 4.54- 4.45 (m, 2H), 4.44- 4.28 (m, 2H), 3.64- 3.35 (m, 4H), 3.31- 3.09 (m, 3H), 2.36- |
| | | | | 2.12 (m, 2H), 2.02- |
| | | | | 1.73 (m, 7H), 1.71- |
| | | | | 1.58 (m, 2H), 1.58- |
| | | | | 1.45 (m, 1H) |
| 616 | ((2-(((3S,6S,10aS)- 3-(3-(2,2- difluoroethoxy)-3- (pyridin-3- yl)azetidine-1- carbonyl)-5- oxodecahydropyrrolo [1,2-a]azocin-6- yl)carbamoyl)benzo [b]thiophen-5- yl)fluoromethyl) phosphonic acid | 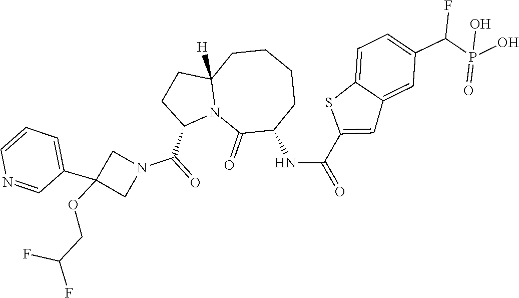 [SMILES: O=C(N[C@H]1CCCC[C@H]2CC[C@@H](C(=O)N3CC(OCC(F)F)(c4cccnc4)C3)N2C1=O)c1cc2cc(C(F)P(=O)(O)O)ccc2s1] | 695.2 | ¹H NMR (400 MHz, CD₃OD) δ 8.90-8.80 (m, 1H), 8.73-8.61 (m, 1H), 8.49-8.36 (m, 1H), 8.07-7.95 (m, 2H), 7.94-7.77 (m, 2H), 7.61-7.53 (m, 1H), 6.17-5.95 (m, 1H), 5.90-5.70 (m, 1H), 5.05-4.94 (m, 2H), 4.70-4.59 (m, 1H), 4.52-4.23 (m, 4H), 3.70-3.56 (m, 2H), 2.36-2.16 (m, 2H), 2.13-1.76 (m, 8H), 1.74-1.56 (m, 2H) |
| 617 | (fluoro(2- (((3S,6S,9aS)-3-(3- (1-methyl-4-oxo- 1,4-dihydropyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 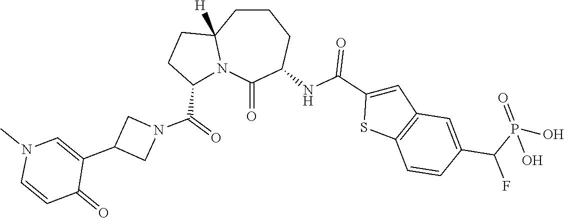 [SMILES: Cn1ccc(=O)c(C2CN(C(=O)[C@@H]3CC[C@@H]4CCC[C@H](NC(=O)c5cc6cc(C(F)P(=O)(O)O)ccc6s5)C(=O)N43)C2)c1] | 631.2 | ¹H NMR (400 MHz, CD₃OD) δ 8.19 (s, 1H), 7.84-7.80 (m, 1H), 7.61-7.46 (m, 2H), 7.45-7.36 (m, 1H), 6.02 (s, 1H), 5.86-5.74 (m, 1H), 5.73-5.59 (m, 1H), 4.78-4.70 (m, 1H), 4.61-4.42 (m, 2H), 4.25 (m, 2H), 4.22- 4.08 (m, 2H), 4.01- 3.90 (m, 1H), 3.74 (s, |
| | | | | 3H), 2.40-2.30 (m, |
| | | | | 1H), 2.22-2.13 (m, |
| | | | | 2H), 2.05-2.00 (m, |
| | | | | 2H), 2.00-1.91 (m, |
| | | | | 2H), 1.89-1.79 (m, |
| | | | | 2H), 1.75-1.60 (m, |
| | | | | 1H) |
| 618 | ((2-(((3S,6S,9aS)-3- (3-(4-ethylpyridin- 3-yl)azetidine-1- carbonyl)-5- oxooctahydro-1H- pyrrolo[1,2- a]azepin-6- yl)carbamoyl)pyraz olo[1,5-a]pyridin-5- yl)methyl) phosphonic acid | 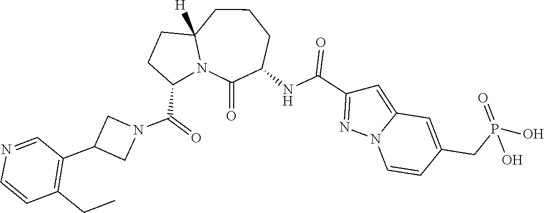 [SMILES: CCc1ccncc1C1CN(C(=O)[C@@H]2CC[C@@H]3CCC[C@H](NC(=O)c4cc5cc(CP(=O)(O)O)ccn5n4)C(=O)N32)C1] | 595.2 | ¹H NMR (400 MHz, CD₃OD) δ 8.97-8.82 (m, 1H), 8.65 (d, J = 6.0 Hz, 1H), 8.51- 8.41 (m, 1H), 7.91 (d, J = 6.0 Hz, 1H), 7.56 (d, J = 2.0 Hz, 1H), 7.01-6.89 (m, 2H), 4.83-4.71 (m, 2H), 4.69-4.55 (m, 2H), 4.51-4.34 (m, 2H), 4.22-4.04 (m, 2H), 3.23-3.12 (m, 2H), |
| | | | | 2.93-2.82 (m, 2H), |
| | | | | 2.37-2.26 (m, 1H), |
| | | | | 2.22-2.08 (m, 2H), |
| | | | | 2.08-2.00 (m, 2H), |
| | | | | 2.00-1.92 (m, 2H), |
| | | | | 1.90-1.79 (m, 2H), |
| | | | | 1.77-1.67 (m, 1H), |
| | | | | 1.39-1.30 (m, 3H) |
| 619 | (fluoro(2- (((3S,6S,9aS)-3-(3- (2-hydroxypyridin- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 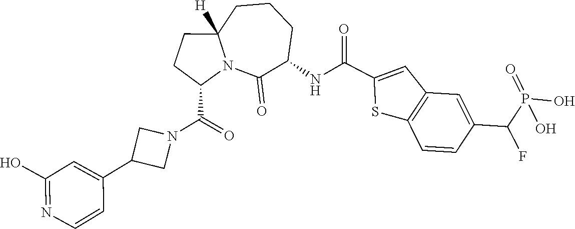 [SMILES: O=C(N[C@H]1CCC[C@H]2CC[C@@H](C(=O)N3CC(c4ccnc(O)c4)C3)N2C1=O)c1cc2cc(C(F)P(=O)(O)O)ccc2s1] | 617.2 | ¹H NMR (400 MHz, DMSO-d₆) δ 8.78- 8.60 (m, 1H), 8.31- 8.15 (m, 1H), 8.06- 7.90 (m, 1H), 7.53- 7.41 (m, 1H), 7.35- 7.11 (m, 3H), 6.40- 6.16 (m, 1H), 5.70- 5.38 (m, 1H), 4.74- 4.61 (m, 1H), 4.50- 4.30 (m, 2H), 4.22- 4.09 (m, 1H), 4.02- 3.95 (m, 1H), 3.79- 3.65 (m, 3H), 2.23- |
| | | | | 2.12 (m, 1H), 2.03- |
| | | | | 1.89 (m, 3H), 1.87- |
| | | | | 1.64 (m, 6H) |
| 620 | (fluoro(2- (((3S,6S,9aS)-5- oxo-3-(3-(4- oxopyrimidin- 1(4H)-yl)azetidine- 1-carbonyl) octahydro- 1H-pyrrolo[1,2- a]azepin-6- yl)carbamoyl)benzo [b]thiophen-5- yl)methyl) phosphonic acid | 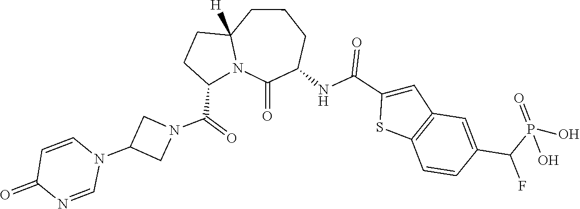 [SMILES: O=C(N[C@H]1CCC[C@H]2CC[C@@H](C(=O)N3CC(n4ccc(=O)nc4)C3)N2C1=O)c1cc2cc(C(F)P(=O)(O)O)ccc2s1] | 618.2 | ¹H NMR (400 MHz, CD₃OD) δ 8.81 (d, J = 10.0 Hz, 1H), 8.60- 8.48 (m, 1H), 8.11- 8.05 (m, 1H), 8.05- 7.99 (m, 1H), 7.98- 7.91 (m, 1H), 7.60 (t, J = 8.4 Hz, 1H), 7.13- 7.01 (m, 1H), 5.94- 5.74 (m, 1H), 5.64- 5.49 (m, 1H), 4.80- 4.72 (m, 2H), 4.62- 4.48 (m, 2H), 4.46- |
| | | | | 4.32 (m, 1H), 4.16- |
| | | | | 4.01 (m, 2H), 2.39- |
| | | | | 2.26 (m, 1H), 2.19- |
| | | | | 2.08 (m, 2H), 2.05- |
| | | | | 1.91 (m, 4H), 1.90- |
| | | | | 1.79 (m, 3H) |
| 621 | ((2-(((3S,6S,10aS)- 3-(3-(2,2- difluoroethoxy)-3- (pyridin-2- yl)azetidine-1- carbonyl)-5- oxodecahydropyrrolo [1,2-a]azocin-6- yl)carbamoyl)benzo [b]thiophen-5- yl)fluoromethyl) phosphonic acid | 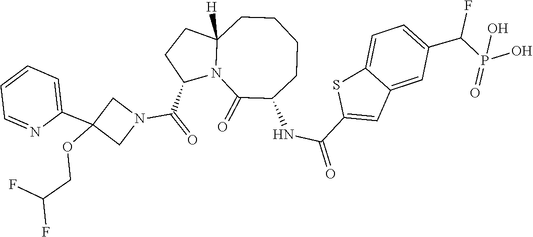 [SMILES: O=C(N[C@H]1CCCC[C@H]2CC[C@@H](C(=O)N3CC(OCC(F)F)(c4ccccn4)C3)N2C1=O)c1cc2cc(C(F)P(=O)(O)O)ccc2s1] | 695.1 | ¹H NMR (400 MHz, CD₃OD) δ 8.70-8.50 (m, 1H), 8.11-7.82 (m, 4H), 7.69 (d, J = 8.4, 1H), 7.61-7.52 (m, 1H), 7.46 (d, J = 5.2, 1H), 6.14-5.91 (m, 1H), 5.90-5.73 (m, 1H), 5.08-4.92 (m, 2H), 4.86-4.72 (m, 1H), 4.64-4.55 (m, 1H), 4.54-4.47 (m, 1H), 4.41 (d, J = 13.2 Hz, 1H), 4.37- 4.20 (m, 1H), 3.68- |
| | | | | 3.45 (m, 2H), 2.38- |
| | | | | 2.17 (m, 2H), 2.14- |
| | | | | 1.91 (m, 5H), 1.91- |
| | | | | 1.75 (m, 3H), 1.74- |
| | | | | 1.54 (m, 2H) |
| 622 | (fluoro(2- (((3S,6S,10aS)-3-(3- (2-methoxyethoxy)- 3-(pyridin-3- yl)azetidine-1- carbonyl)-5- oxodecahydropyrrolo [1,2-a]azocin-6- yl)carbamoyl)benzo [b]thiophen-5- yl)methyl) phosphonic acid | 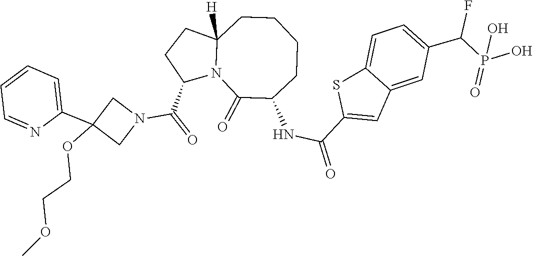 [SMILES: COCCOC1(c2ccccn2)CN(C(=O)[C@@H]2CC[C@@H]3CCCC[C@H](NC(=O)c4cc5cc(C(F)P(=O)(O)O)ccc5s4)C(=O)N32)C1] | 689.2 | ¹H NMR (400 MHz, CD₃OD) δ 8.96-8.82 (m, 1H), 8.69-8.35 (m, 2H), 8.10-7.96 (m, 2H), 7.93-7.75 (m, 2H), 7.63-7.53 (m, 1H), 5.87-5.66 (m, 1H), 5.06-4.95 (m, 1H), 4.70-4.60 (m, 1H), 4.59-4.38 (m, 3H), 4.38-4.15 (m, 2H), 3.62-3.45 (m, 4H), 3.37 (s, 3H), 2.37-2.17 (m, 2H), 2.11-1.78 (m, 8H), 1.75-1.56 (m, 2H) |
| 623 | (fluoro(2- (((3S,6S,9aS)-3-(3- (3-fluoropyridin-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 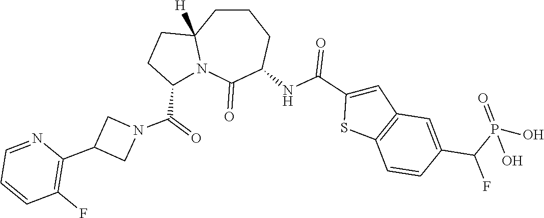 [SMILES: O=C(N[C@H]1CCC[C@H]2CC[C@@H](C(=O)N3CC(c4ncccc4F)C3)N2C1=O)c1cc2cc(C(F)P(=O)(O)O)ccc2s1] | 619.2 | ¹H NMR (400 MHz, CD₃OD) δ 8.51-8.35 (m, 1H), 8.16-7.86 (m, 3H), 7.65-7.48 (m, 2H), 7.45-7.29 (m, 1H), 5.95-5.74 (m, 1H), 4.80-4.70 (m, 2H), 4.67-4.51 (m, 2H), 4.46-4.19 (m, 3H), 4.13-4.00 (m, 1H), 2.38-2.25 (m, 1H), 2.20-1.73 (m, 9H) |
| 624 | (fluoro(2- (((3S,6S,9aS)-3-(3- (1-methyl-6-oxo-1,6- dihydropyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 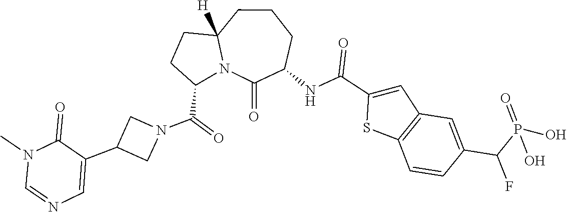 [SMILES: Cn1cncc(C2CN(C(=O)[C@@H]3CC[C@@H]4CCC[C@H](NC(=O)c5cc6cc(C(F)P(=O)(O)O)ccc6s5)C(=O)N43)C2)c1=O] | 632.1 | ¹H NMR (400 MHz, CD₃OD) δ 8.36 (s, 1H), 8.08-8.01 (m, 2H), 7.94-7.80 (m, 2H), 7.68-7.58 (m, 1H), 5.75-5.59 (m, 1H), 4.75-4.70 (m, 2H), 4.58 (d, J = 7.4 Hz, 1H), 4.44-4.43 (m, 1H), 4.48-4.41 (m, 1H), 4.18-4.04 (m, 2H), 3.96-3.84 (m, 1H), 3.52 (d, J = 13.2 Hz, 3H), 2.33- |
| | | | | 2.24 (m, 1H), 2.10- |
| | | | | 1.99 (m, 3H), 1.98- |
| | | | | 1.86 (m, 4H), 1.83 |
| | | | | (dd, J = 0.8, 10.2 Hz, |
| | | | | 2H) |
| 625 | (fluoro(2- (((3S,6S,9aS)-3-(3- (5-fluoro-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 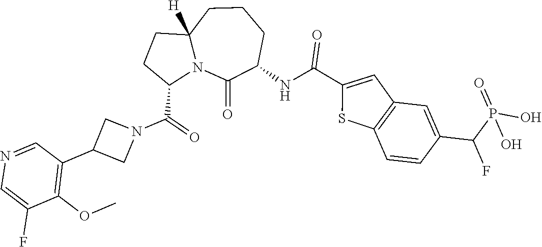 [SMILES: COc1c(F)cncc1C1CN(C(=O)[C@@H]2CC[C@@H]3CCC[C@H](NC(=O)c4cc5cc(C(F)P(=O)(O)O)ccc5s4)C(=O)N32)C1] | 649.2 | ¹H NMR (400 MHz, CD₃OD) δ 8.53-8.44 (m, 1H), 8.36-8.26 (m, 1H), 8.07-8.00 (m, 1H), 8.00-7.94 (m, 1H), 7.94-7.89 (m, 1H), 7.63-7.53 (m, 1H), 5.89-5.71 (m, 1H), 4.74 (d, J = 8.8 Hz, 2H), 4.64 (d, J = 7.6 Hz, 2H), 4.56- 4.52 (m, 1H), 4.35- 4.30 (m, 3H), 4.20- 4.00 (m, 3H), 2.35- 2.27 (m, 1H), 2.14- |
| | | | | 2.06 (m, 2H), 2.04- |
| | | | | 1.99 (m, 1H), 1.97- |
| | | | | 1.88 (m, 3H), 1.87- |
| | | | | 1.78 (m, 3H) |
| 626 | (fluoro(2- (((3S,6S,9aS)-3-(3-(4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 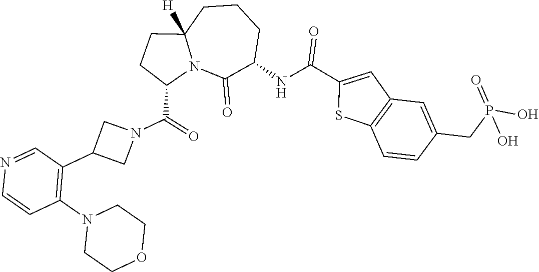 [SMILES: O=C(N[C@H]1CCC[C@H]2CC[C@@H](C(=O)N3CC(c4cnccc4N4CCOCC4)C3)N2C1=O)c1cc2cc(CP(=O)(O)O)ccc2s1] | 686.2 | ¹H NMR (400 MHz, CD₃OD) δ 8.36-8.14 (m, 1H), 8.14-7.94 (m, 1H), 7.92-7.83 (m, 1H), 7.82-7.69 (m, 2H), 7.61-7.43 (m, 1H), 7.26-6.88 (m, 1H), 5.86-5.63 (m, 1H), 4.81-4.67 (m, 2H), 4.59-4.47 (m, 2H), 4.43-4.25 (m, 1H), 4.18-4.00 (m, 3H), 3.78 (d, J = 2.8 Hz, 4H), 3.35 (s, 1H), 3.22 (s, 3H), 2.36-2.30 (m, 1H), 2.21-2.08 (m, 2H), |
| | | | | 2.06-1.79 (m, 7H) |
| 627 | (fluoro(2- (((3S,6S,9aS)-3-(3- ((methylsulfonyl) methyl)azetidine-1- carbonyl)-5- oxooctahydro-1H- pyrrolo[1,2- a]azepin-6- yl)carbamoyl)benzo [b]thiophen-5- yl)methyl) phosphonic acid | 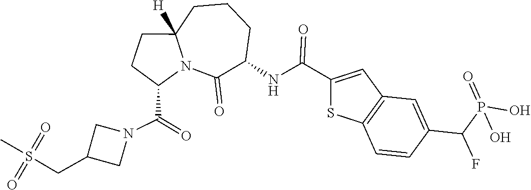 [SMILES: CS(=O)(=O)CC1CN(C(=O)[C@@H]2CC[C@@H]3CCC[C@H](NC(=O)c4cc5cc(C(F)P(=O)(O)O)ccc5s4)C(=O)N32)C1] | 616.3 | ¹H NMR (400 MHz, CD₃OD) δ 8.08 (s, 1H), 8.03 (s, 1H), 7.95 (d, J = 8.8 Hz, 1H), 7.59 (d, J = 8.4 Hz, 1H), 5.94-5.60 (m, 1H), 4.76-4.66 (m, 1H), 4.56-4.13 (m, 4H), 4.13-3.99 (m, 1H), 3.98-3.81 (m, 1H), 3.61-3.49 (m, 2H), 3.29-3.19 |
| | | | | (m, 1H), 3.31-2.97 |
| | | | | (m, 3H), 2.35-2.22 |
| | | | | (m, 1H), 2.12-1.77 |
| | | | | (m, 9H) |
| 628 | (fluoro(2- (((3S,6S,9aS)-3-(3- (1-methyl-2-oxo- 1,2-dihydropyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 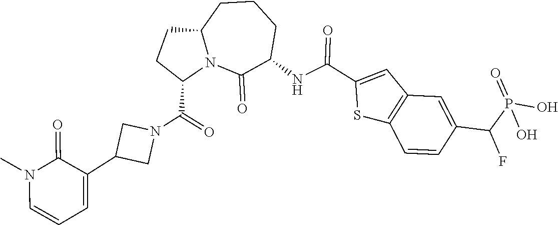 | 631.1 | ¹H NMR (400 MHz, CD₃OD) δ 8.10-7.97 (m, 2H), 7.90-7.83 (m, 1H), 7.68-7.45 (m, 3H), 6.42-6.33 (m, 1H), 5.78-5.50 (m, 1H), 4.80-4.69 (m, 2H), 4.61-4.50 (m, 2H), 4.40-4.30 (m, 1H), 4.14-3.92 (m, 3H), 3.58-3.53 (m, 3H), 2.34-2.23 (m, 1H), 2.13-1.78 (m, 9H) |
| 629 | (fluoro(2- (((3S,6S,9aS)-3-(3- (5-methoxy-1- methyl-1H-pyrazol- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 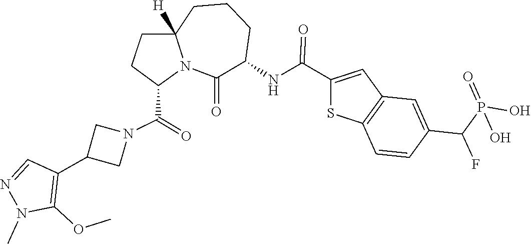 [SMILES: COc1c(C2CN(C(=O)[C@@H]3CC[C@@H]4CCC[C@H](NC(=O)c5cc6cc(C(F)P(=O)(O)O)ccc6s5)C(=O)N43)C2)cnn1C] | 634.3 | ¹H NMR (400 MHz, CD₃OD) δ 8.08-8.07 (m, 1H), 8.03 (s, 1H), 7.96-7.93 (m, 1H), 7.64-7.49 (m, 2H), 5.91-5.75 (m, 1H), 4.88-4.80 (m, 1H), 4.73-4.75 (m, 1H), 4.65-4.52 (m, 2H), 4.52-4.37 (m, 1H), 4.35-4.20 (m, 1H), 4.11-4.03 (m, 1H), 3.98-3.95 (m, 1H), 3.94-3.91 (m, 3H), 3.65-3.63 (m, 3H), |
| | | | | 2.36-2.25 (m, 1H), |
| | | | | 2.17-2.05 (m, 2H), |
| | | | | 2.04-1.81 (m, 7H) |
| 630 | (fluoro(2- (((3S,6S,9aS)-5- oxo-3-(1′H- spiro[azetidine-3,3′- furo [3,4- c]pyridine]-1- carbonyl)octahydro- 1H-pyrrolo[1,2- a]azepin-6- yl)carbamoyl)benzo [b]thiophen-5- yl)methyl) phosphonic acid | 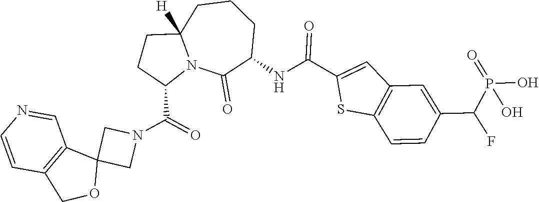 [SMILES: O=C(N[C@H]1CCC[C@H]2CC[C@@H](C(=O)N3CC4(C3)OCc3ccncc34)N2C1=O)c1cc2cc(C(F)P(=O)(O)O)ccc2s1] | 629.3 | ¹H NMR (400 MHz, DMSO-d₆) δ 9.07- 8.75 (m, 2H), 8.73- 8.53 (m, 1H), 8.37- 8.19 (m, 1H), 8.12- 7.91 (m, 2H), 7.70- 7.44 (m, 2H), 5.94- 5.74 (m, 1H), 5.22- 5.08 (m, 2H), 4.77- 4.64 (m, 2H), 4.56- 4.44 (m, 2H), 4.30- 4.15 (m, 3H), 4.06- 3.95 (m, 2H), 2.36- |
| | | | | 2.14 (m, 1H), 2.12- |
| | | | | 1.61 (m, 9H) |
| 631 | ((S)-(2- (((3S,6S,9aS)-3-(3-(4- (dimethylamino) pyridin-3-yl)azetidine- 1-carbonyl)-5- oxooctahydro-1H- pyrrolo[1,2- a]azepin-6- yl)carbamoyl)benzo [b]thiophen-5- yl)fluoromethyl) phosphonic acid | 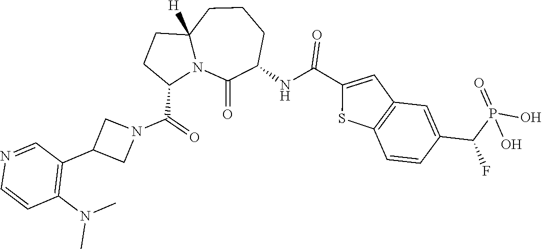 [SMILES: CN(C)c1ccncc1C1CN(C(=O)[C@@H]2CC[C@@H]3CCC[C@H](NC(=O)c4cc5cc([C@@H](F)P(=O)(O)O)ccc5s4)C(=O)N32)C1] | 644.2 | ¹H NMR (400 MHz, CD₃OD) δ 8.27-8.14 (m, 1H), 8.08-7.80 (m, 4H), 7.64-7.51 (m, 1H), 7.07-6.82 (m, 1H), 5.85-5.67 (m, 1H), 4.79-4.66 (m, 2H), 4.59-4.47 (m, 2H), 4.44-4.21 (m, 2H), 4.15-3.98 (m, 2H), 3.14-3.05 (m, 6H), 2.38-2.25 (m, 1H), 2.18-2.06 (m, 2H), 2.04-1.92 (m, 3H), 1.92-1.75 (m, 4H) |
| 632 | ((2-(((3S,6S,9aS)-3- (3-(4- (difluoromethoxy) pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 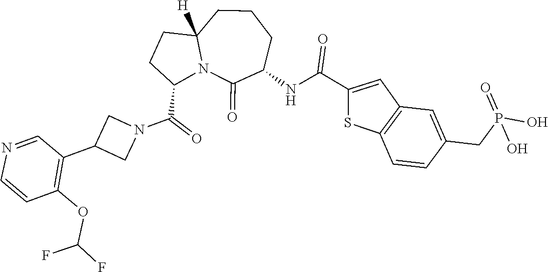 [SMILES: O=C(N[C@H]1CCC[C@H]2CC[C@@H](C(=O)N3CC(c4cnccc4OC(F)F)C3)N2C1=O)c1cc2cc(CP(=O)(O)O)ccc2s1] | 648.6 | ¹H NMR (400 MHz, CD₃OD) δ 8.80-8.67 (m, 1H), 8.66-8.54 (m, 1H), 7.97 (d, J = 10.4 Hz, 1H), 7.89- 7.73 (m, 2H), 7.63- 7.48 (m, 1H), 7.41 (t, J = 7.6 Hz, 1H), 7.37- 7.11 (m, 1H), 4.92 (s, 1H), 4.78-4.64 (m, 2H), 4.58-4.51 (m, 1H), 4.48-4.37 (m, 1H), 4.27-4.13 (m, 2H), 4.07 (d, J = 4.4 Hz, 1H), 3.29- 3.17 (m, 2H), 2.39- 2.24 (m, 1H), 2.20- |
| | | | | 2.05 (m, 2H), 2.04- |
| | | | | 1.96 (m, 2H), 1.95- |
| | | | | 1.90 (m, 2H), 1.89- |
| | | | | 1.77 (m, 3H) |
| 633 | (fluoro(2- (((3S,6S,9aS)-3-(3- (3-methoxy-1H- pyrazol-1- yl)azetidine-1- carbonyl)-5- oxooctahydro-1H- pyrrolo[1,2- a]azepin-6- yl)carbamoyl)benzo [b]thiophen-5- yl)methyl) phosphonic acid | 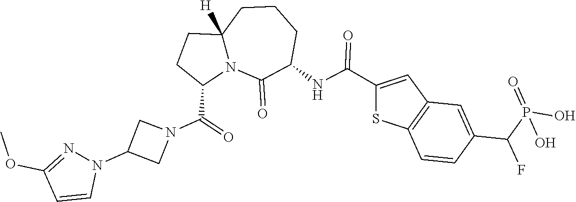 [SMILES: COc1ccn(C2CN(C(=O)[C@@H]3CC[C@@H]4CCC[C@H](NC(=O)c5cc6cc(C(F)P(=O)(O)O)ccc6s5)C(=O)N43)C2)n1] | 620.1 | ¹H NMR (400 MHz, CD₃OD) δ 8.11-8.01 (m, 2H), 7.88 (d, J = 8.4 Hz, 1H), 7.63 (d, J = 6.8 Hz, 1H), 7.55- 7.35 (m, 1H), 5.81- 5.57 (m, 1H), 5.17- 5.05 (m, 1H), 4.74 (d, J = 10.8 Hz, 1H), 4.66- 4.52 (m, 2H), 4.49- 4.40 (m, 1H), 4.40- 4.30 (m, 1H), 4.30- 4.20 (m, 1H), 4.07 (d, |
| | | | | J = 5.2 Hz, 1H), 3.92 |
| | | | | (s, 1H), 3.87 (s, 1H), |
| | | | | 3.83 (s, 1H), 2.36- |
| | | | | 2.26 (m, 1H), 2.16- |
| | | | | 2.10 (m, 1H), 2.09- |
| | | | | 2.03 (m, 2H), 2.02 (s, |
| | | | | 1H), 1.97-1.90 (m, |
| | | | | 2H), 1.89-1.80 (m, |
| | | | | 3H) |
| 634 | ((R)-(2- (((3S,6S,9aS)-3-(3- (4-(dimethylamino) pyridin-3-yl)azetidine- 1-carbonyl)-5- oxooctahydro-1H- pyrrolo[1,2- a]azepin-6- yl)carbamoyl)benzo [b]thiophen-5- yl)fluoromethyl) phosphonic acid | 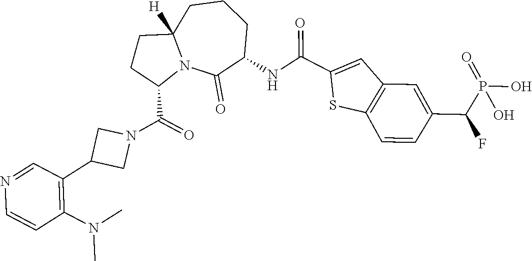 [SMILES: CN(C)c1ccncc1C1CN(C(=O)[C@@H]2CC[C@@H]3CCC[C@H](NC(=O)c4cc5cc([C@H](F)P(=O)(O)O)ccc5s4)C(=O)N32)C1] | 644.5 | ¹H NMR (400 MHz, CD₃OD) δ 8.22-8.09 (m, 1H), 8.06-7.97 (m, 1H), 7.94 (s, 1H), 7.89-7.85 (m, 1H), 7.79 (m, 1H), 7.60- 7.49 (m, 1H), 6.81 (m, 1H), 5.86-5.65 (m, 1H), 4.73 (s, 1H), 4.66 (s, 1H), 4.57- 4.50 (m, 1H), 4.46 (s, 1H), 4.39 (s, 1H), 4.26-4.18 (m, 1H), 4.08 (s, 1H), 4.02- 3.96 (m, 1H), 3.09- 3.02 (m, 6H), 2.36- |
| | | | | 2.26 (m, 1H), 2.15 |
| | | | | (m, 1H), 2.09 (m, |
| | | | | 1H), 2.02-1.96 (m, |
| | | | | 2H), 1.95-1.90 (m, |
| | | | | 2H), 1.88-1.81 (m, |
| | | | | 3H) |
| 635 | (fluoro(2- (((3S,6S,9aS)-3-(3- (5-fluoro-1-methyl- 1H-pyrazol-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 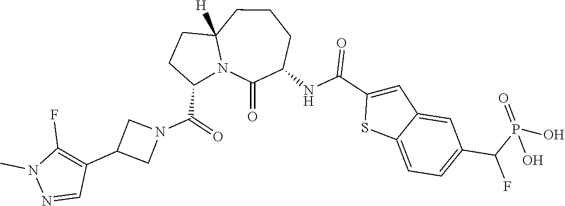 [SMILES: Cn1ncc(C2CN(C(=O)[C@@H]3CC[C@@H]4CCC[C@H](NC(=O)c5cc6cc(C(F)P(=O)(O)O)ccc6s5)C(=O)N43)C2)c1F] | 622.2 | ¹H NMR (400 MHz, CD₃OD) δ 8.11-7.99 (m, 2H), 7.95 (d, J = 8.4 Hz, 1H), 7.64- 7.43 (m, 2H), 5.92- 5.75 (m, 1H), 4.82- 4.72 (m, 2H), 4.62- 4.45 (m, 2H), 4.43- 4.27 (m, 1H), 4.10- 3.92 (m, 2H), 3.85- 3.75 (m, 1H), 3.70 (d, J = 11.1 Hz, 3H), 2.36- 2.23 (m, 1H), 2.15- |
| | | | | 1.79 (m, 9H) |
| 636 | ((R)-fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 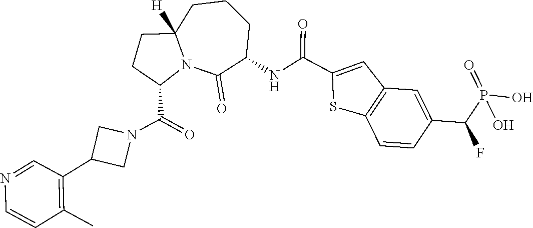 [SMILES: Cc1ccncc1C1CN(C(=O)[C@@H]2CC[C@@H]3CCC[C@H](NC(=O)c4cc5cc([C@H](F)P(=O)(O)O)ccc5s4)C(=O)N32)C1] | 615.2 | ¹H NMR (400 MHz, CD₃OD) δ 8.71-8.47 (m, 1H), 8.41-8.16 (m, 1H), 8.03-7.60 (m, 4H), 7.55 (d, J = 8.0 Hz, 1H), 5.88- 5.67 (m, 1H), 4.74 (d, J = 11.2 Hz, 2H), 4.63- 4.48 (m, 2H), 4.46- 4.29 (m, 1H), 4.27- 3.98 (m, 3H), 2.50- 2.37 (m, 3H), 2.36- 2.26 (m, 1H), 2.21- 2.06 (m, 2H), 2.05- |
| | | | | 1.93 (m, 3H), 1.92- |
| | | | | 1.76 (m, 4H) |
| 637 | ((R)-fluoro(2- (((3S,6S,9aS)-3- (((3S,4S)-4-fluoro- 1-(1H-imidazol-2- yl)pyrrolidin-3- yl)carbamoyl)-5- oxooctahydro-1H- pyrrolo[1,2- a]azepin-6- yl)carbamoyl)benzo [b]thiophen-5- yl)methyl) phosphonic acid | 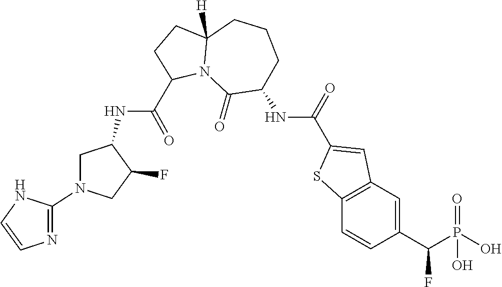 [SMILES: O=C(N[C@H]1CCC[C@H]2CCC(C(=O)N[C@H]3CN(c4ncc[nH]4)C[C@@H]3F)N2C1=O)c1cc2cc([C@H](F)P(=O)(O)O)ccc2s1] | 637.2 | ¹H NMR (400 MHz, CD₃OD) δ 8.00 (s, 2H), 7.89 (d, J = 8.4 Hz, 1H), 7.59 (d, J = 8.8 Hz, 1H), 6.88 (s, 2H), 5.82-5.68 (m, 1H), 5.29-5.16 (m, 1H), 4.70 (d, J = 11.6 Hz, 1H), 4.62-4.58 (m, 1H), 4.43-4.40 (m, 1H), 4.07-4.05 (m, 1H), 3.94-3.72 (m, 3H), 3.61 (d, J = 10.4 Hz, 1H), 2.31- 2.27 (m, 1H), 2.13- 2.07 (m, 3H), 2.04- 1.79 (m, 6H) |
| 638 | ((2- (((3S,6S,8R,9aR)-3- (3-(4- (dimethylamino) pyridin-3- yl)azetidine- 1-carbonyl)-8- methyl-5- oxooctahydro-1H- pyrrolo[1,2- a]azepin-6- yl)carbamoyl)benzo [b]thiophen-5- yl)methyl) phosphonic acid | 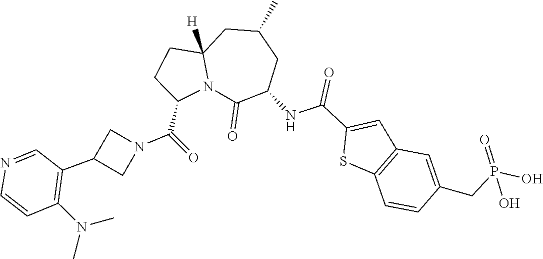 [SMILES: C[C@H]1C[C@H]2CC[C@@H](C(=O)N3CC(c4cnccc4N(C)C)C3)N2C(=O)[C@@H](NC(=O)c2cc3cc(CP(=O)(O)O)ccc3s2)C1] | 640.3 | ¹H NMR (400 MHz, DMSO-d₆) δ 8.73- 8.60 (m, 1H), 8.26- 8.21 (m, 1H), 8.14 (d, J = 17.2 Hz, 1H), 7.90 (d, J = 3.6 Hz, 1H), 7.77 (d, J = 8.8 Hz, 1H), 7.39-7.32 (m, 1H), 7.07 (t, J = 7.2 Hz, 1H), 4.77 (t, J = 8.0 Hz, 1H), 4.69- 4.58 (m, 2H), 4.46- 4.34 (m, 2H), 4.30- 4.20 (m, 3H), 4.01- 3.94 (m, 2H), 3.12 (s, 2H), 3.07 (d, J = 4.0 |
| | | | | Hz, 6H), 2.27-2.15 |
| | | | | (m, 1H), 2.07-1.90 |
| | | | | (m, 2H), 1.87-1.75 |
| | | | | (m, 3H), 1.69 (d, J = |
| | | | | 13.2 Hz, 1H), 1.55- |
| | | | | 1.34 (m, 2H), 0.95 (d, |
| | | | | J = 4.8 Hz, 3H) |
| 639 | ((2- (((3S,6S,8S,9aR)-3- (3-(4- (dimethylamino) pyridin-3-yl)azetidine- 1-carbonyl)-8- methyl-5- oxooctahydro-1H- pyrrolo[1,2- a]azepin-6- yl)carbamoyl)benzo [b]thiophen-5- yl)methyl) phosphonic acid | 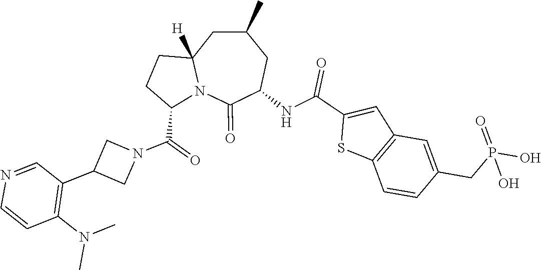 [SMILES: C[C@@H]1C[C@H]2CC[C@@H](C(=O)N3CC(c4cnccc4N(C)C)C3)N2C(=O)[C@@H](NC(=O)c2cc3cc(CP(=O)(O)O)ccc3s2)C1] | 640.1 | ¹H NMR (400 MHz, CD₃OD) δ 8.23-8.12 (m, 1H), 8.00-7.93 (m, 1H), 7.81-7.67 (m, 3H), 7.35-7.33 (m, 1H), 7.03-6.83 (m, 1H), 5.00-4.97 (m, 1H), 4.67 (t, J = 8.8 Hz, 1H), 4.50- 4.47 (m, 2H), 4.38 (t, J = 8.8 Hz, 1H), 4.29- 4.25 (m, 2H), 4.02- 3.98 (m, 1H), 3.31- 3.13 (m, 2H), 3.08- 3.04 (m, 6H), 2.32- 2.29 (m, 2H), 2.21- |
| | | | | 2.10 (m, 1H), 2.05- |
| | | | | 1.99 (m, 3H), 1.93- |
| | | | | 1.87 (m, 1H), 1.82- |
| | | | | 1.78 (m, 1H), 1.62- |
| | | | | 1.58 (m, 1H), 1.23- |
| | | | | 1.21 (m, 3H) |
| 640 | ((2-(((3S,6S,10aS)- 3-(((3H- imidazo[4,5- b]pyridin-6- yl)methyl) carbamoyl)-5- oxodecahydropyrrolo [1,2-a]azocin-6- yl)carbamoyl)benzo [b]thiophen-5- yl)methyl) phosphonic acid | 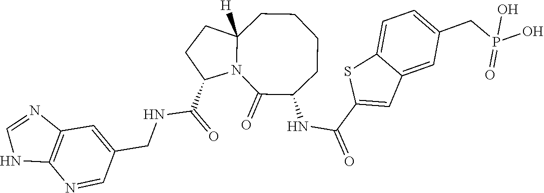 [SMILES: O=C(N[C@H]1CCCC[C@H]2CC[C@@H](C(=O)NCc3cnc4[nH]cnc4c3)N2C1=O)c1cc2cc(CP(=O)(O)O)ccc2s1] | 611.0 | ¹H NMR (400 MHz, CD₃OD) δ 8.84 (s, 1H), 8.58 (s, 1H), 8.28 (s, 1H), 7.99 (s, 1H), 7.92-7.77 (m, 2H), 7.44 (d, J = 8.4 Hz, 1H), 5.05-4.99 (m, 1H), 4.75 (s, 1H), 4.57-4.38 (m, 3H), 3.29 (s, 1H), 3.24 (s, 1H), 2.35-2.20 (m, 2H), 2.15-2.07 (m, |
| | | | | 1H), 2.05-1.90 (m, |
| | | | | 5H), 1.87-1.83 (m, |
| | | | | 1H), 1.80-1.57 (m, |
| | | | | 3H) |
| 641 | (difluoro(2- (((3S,6S,10aR,Z)-9- methyl-3-(3-(4- methylpyridin-3- yl)azetidine-1- carbonyl)-5-oxo- 1,2,3,5,6,7,10,10a- octahydropyrrolo[1, 2-a]azocin-6- yl)carbamoyl)benzo [b]thiophen-5- yl)methyl) phosphonic acid | 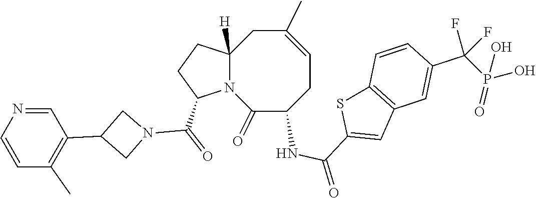 [SMILES: C/C1=C/C[C@H](NC(=O)c2cc3cc(C(F)(F)P(=O)(O)O)ccc3s2)C(=O)N2[C@H](CC[C@H]2C(=O)N2CC(c3cnccc3C)C2)C1] | 659.1 | ¹H NMR (400 MHz, DMSO-d₆) δ 8.94- 8.85 (m, 1H), 8.36- 8.27 (m, 1H), 8.16- 8.00 (m, 2H), 7.98- 7.91 (m, 1H), 7.62- 7.50 (m, 1H), 7.33- 7.16 (m, 1H), 5.58- 5.50 (m, 1H), 5.03- 4.95 (m, 1H), 4.62 (t, J = 8.0 Hz, 1H), 4.47- 4.39 (m, 1H), 4.37- 4.32 (m, 1H), 4.30- |
| | | | | 4.24 (m, 1H), 4.18 (t, |
| | | | | J = 8.4 Hz, 1H), 4.09- |
| | | | | 3.95 (m, 2H), 2.81- |
| | | | | 2.65 (m, 1H), 2.60- |
| | | | | 2.50 (m, 3H), 2.40- |
| | | | | 2.24 (m, 2H), 2.23 (s, |
| | | | | 1H), 2.09 (s, 3H), |
| | | | | 1.98-1.89 (m, 1H), |
| | | | | 1.82 (s, 3H) |
| 642 | ((2- (((3S,6S,10aR,Z)-9- methyl-3-(3-(4- methylpyridin-3- yl)azetidine-1- carbonyl)-5-oxo- 1,2,3,5,6,7,10,10a- octahydropyrrolo[1, 2-a]azocin-6- yl)carbamoyl)benzo [b]thiophen-5- yl)methyl) phosphonic acid | 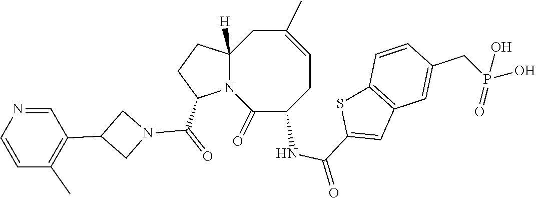 [SMILES: C/C1=C/C[C@H](NC(=O)c2cc3cc(CP(=O)(O)O)ccc3s2)C(=O)N2[C@H](CC[C@H]2C(=O)N2CC(c3cnccc3C)C2)C1] | 623.2 | ¹H NMR (400 MHz, CD₃OD) δ 8.39 (s, 1H), 8.09-8.02 (m, 1H), 7.87-7.78 (m, 1H), 7.73-7.60 (m, 2H), 7.44-7.27 (m, 2H), 5.62 (t, J = 6.4 Hz, 1H), 5.12-5.01 (m, 1H), 4.75-4.51 (m, 2H), 4.48-4.42 (m, 1H), 4.42-4.37 (m, 1H), 4.36-4.20 (m, 1H), 4.19-4.01 |
| | | | | (m, 2H), 3.19-3.08 |
| | | | | (m, 2H), 2.83-2.64 |
| | | | | (m, 2H), 2.56-2.38 |
| | | | | (m, 2H), 2.27 (d, J = |
| | | | | 9.6 Hz, 3H), 2.22- |
| | | | | 2.08 (m, 3H), 2.03- |
| | | | | 1.95 (m, 1H), 1.91 ( d, |
| | | | | J = 12.0 Hz, 3H) |
| 643 | (difluoro(2- (((3S,6S,10aR,Z)-9- methyl-3-(3-(4- methylpyridin-3- yl)azetidine-1- carbonyl)-5-oxo- 1,2,3,5,6,7,8,10a- octahydropyrrolo[1, 2-a]azocin-6- yl)carbamoyl)benzo [b]thiophen-5- yl)methyl) phosphonic acid | 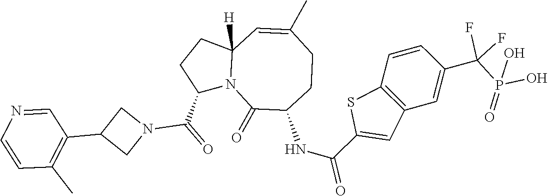 [SMILES: C/C1=C/[C@H]2CC[C@@H](C(=O)N3CC(c4cnccc4C)C3)N2C(=O)[C@@H](NC(=O)c2cc3cc(C(F)(F)P(=O)(O)O)ccc3s2)CC1] | 658.9 | ¹H NMR (400 MHz, DMSO-d₆) δ 8.99 (dd, J = 7.2, 16.0 Hz, 1H), 8.61-8.46 (m, 1H), 8.37-8.24 (m, 2H), 8.14-8.01 (m, 2H), 7.62-7.55 (m, 1H), 7.27-7.20 (m, 1H), 5.33 (s, 1H), 4.86-4.79 (m, 1H), 4.58 (s, 1H), 4.56- 4.43 (m, 2H), 4.38- 4.18 (m, 2H), 4.14- |
| | | | | 4.07 (m, 1H), 4.05- |
| | | | | 3.97 (m, 1H), 2.47- |
| | | | | 2.40 (m, 1H), 2.35- |
| | | | | 2.24 (m, 1H), 2.21 (d, |
| | | | | J = 1.6 Hz, 3H), 2.07- |
| | | | | 1.88 (m, 3H), 1.84 |
| | | | | (td, J = 5.6, 11.6 Hz, |
| | | | | 2H), 1.77 (s, 3H), |
| | | | | 1.68-1.60 (m, 1H) |
| 644 | ((2- (((3S,6S,10aR,Z)-9- methyl-3-(3-(4- methylpyridin-3- yl)azetidine-1- carbonyl)-5-oxo- 1,2,3,5,6,7,8,10a- octahydropyrrolo[1, 2-a]azocin-6- yl)carbamoyl)benzo [b]thiophen-5- yl)methyl) phosphonic acid | 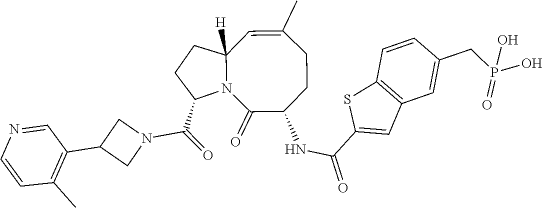 [SMILES: C/C1=C/[C@H]2CC[C@@H](C(=O)N3CC(c4cnccc4C)C3)N2C(=O)[C@@H](NC(=O)c2cc3cc(CP(=O)(O)O)ccc3s2)CC1] | 623.2 | ¹H NMR (400 MHz, CD₃OD) δ 8.53-8.38 (m, 1H), 8.12-7.97 (m, 1H), 7.82 (s, 1H), 7.77-7.64 (m, 2H), 7.42 (d, J = 8.4 Hz, 1H), 7.33 (d, J = 5.6 Hz, 1H), 5.41 (s, 1H), 5.01-4.88 (m, 2H), 4.77-4.52 (m, 4H), 4.24-4.17 (m, 1H), 4.13-4.06 (m, 1H), 3.22-3.10 (m, 2H), |
| | | | | 2.32 (s, 1H), 2.29 (s, |
| | | | | 3H), 2.19-2.10 (m, |
| | | | | 2H), 2.10-2.01 (m, |
| | | | | 2H), 2.01-1.89 (m, |
| | | | | 2H), 1.85 (s, 3H), |
| | | | | 1.82 (s, 1H) |
| 645 | (difluoro(2- (((3S,6S,10aS)-3- ((2-hydroxy-1-(1H- pyrrolo[2,3- b]pyridin-3- yl)ethyl) carbamoyl)-5- oxodecahydropyrrolo [1,2-a]azocin-6- yl)carbamoyl)benzo [b]thiophen-5- yl)methyl) phosphonic acid | 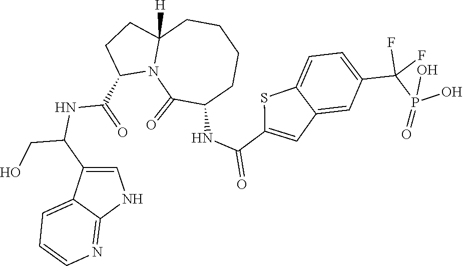 [SMILES: O=C(N[C@H]1CCCC[C@H]2CC[C@@H](C(=O)NC(CO)c3c[nH]c4ncccc34)N2C1=O)c1cc2cc(C(F)(F)P(=O)(O)O)ccc2s1] | 676.0 | ¹H NMR (400 MHz, CD₃OD) δ 8.74-8.50 (m, 1H), 8.20 (d, J = 5.6 Hz, 1H), 8.13- 8.03 (m, 1H), 7.96 (s, 1H), 7.84 (d, J = 8.8 Hz, 1H), 7.71-7.63 (m, 2H), 7.42-7.29 (m, 1H), 5.35-5.25 (m, 1H), 5.02-4.94 (m, 1H), 4.54-4.45 (m, 1H), 4.44-4.36 (m, 1H), 3.92 (d, J = 6.0 Hz, 2H), 2.33- 2.09 (m, 3H), 1.99- 1.81 (m, 6H), 1.76- 1.56 (m, 3H) |
| 646 | ((2-(((3S,6S,9aS)-3- (3-(4-methyl-6-oxo- 1,6-dihydropyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 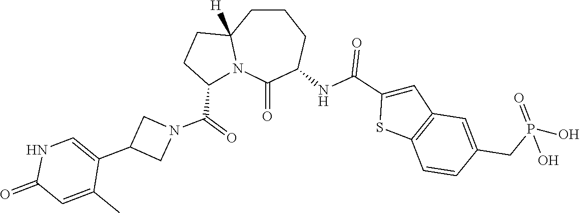 [SMILES: Cc1cc(=O)[nH]cc1C1CN(C(=O)[C@@H]2CC[C@@H]3CCC[C@H](NC(=O)c4cc5cc(CP(=O)(O)O)ccc5s4)C(=O)N32)C1] | 613.1 | ¹H NMR (400 MHz, DMSO-d₆) δ 8.71 (d, J = 8.00 Hz, 1H) 8.17 (d, J = 8.00 Hz, 1H) 7.82-7.97 (m, 1H) 7.78 (s, 1H) 7.19 (s, 2H) 6.21 (d, J = 4.00 Hz, 1H) 4.64 (s, 2H) 4.40-4.49 (m, 2H) 4.25-4.34 (m, 2H) 4.16 (s, 1H) 3.83 (s, 1H) 3.06-3.13 (m, 2H) 2.15-2.23 (m, |
| | | | | 1H) 1.93-2.04 (m, |
| | | | | 5H) 1.66-1.87 (m, |
| | | | | 7H |
| 647 | ((2-(((3S,6S,10aS)- 3-(3-(4- methylpyridin-3- yl)azetidine-1- carbonyl)-5- oxodecahydropyrrolo [1,2-a]azocin-6- yl)carbamoyl)benzo [b]thiophen-5- yl)methyl) phosphonic acid | 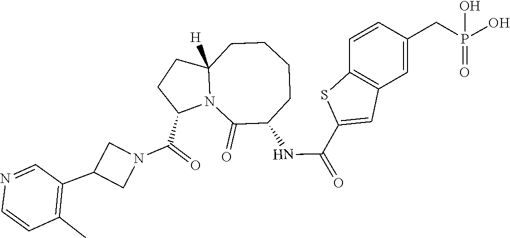 [SMILES: Cc1ccncc1C1CN(C(=O)[C@@H]2CC[C@@H]3CCCC[C@H](NC(=O)c4cc5cc(CP(=O)(O)O)ccc5s4)C(=O)N32)C1] | 611.0 | ¹H NMR (400 MHz, CD₃OD) δ 8.76-8.66 (m, 1H), 8.49-8.37 (m, 1H), 7.96 (s, 1H), 7.82-7.66 (m, 3H), 7.43-7.32 (m, 1H), 5.03-4.93 (m, 1H), 4.79-4.53 (m, 2H), 4.52-4.39 (m, 3H), 4.30-4.22 (m, 1H), 4.21-4.08 (m, 1H), 3.26-3.14 (m, 2H), 2.46 (s, 3H), 2.35- 2.16 (m, 2H), 2.12- 1.83 (m, 8H), 1.75- |
| | | | | 1.54 (m, 2H) |
| 648 | (difluoro(2- (((3S,6S,9aS)-3-(3- (3-methylpyrazin-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 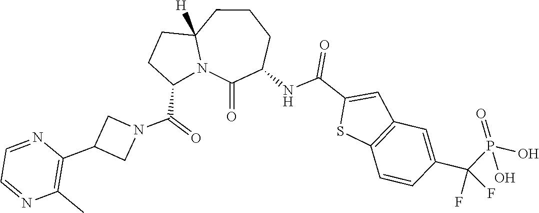 [SMILES: Cc1nccnc1C1CN(C(=O)[C@@H]2CC[C@@H]3CCC[C@H](NC(=O)c4cc5cc(C(F)(F)P(=O)(O)O)ccc5s4)C(=O)N32)C1] | 634.1 | ¹H NMR (400 MHz, CD₃OD) δ 8.47 (d, J = 2.4 Hz, 1H), 8.36- 8.27 (m, 1H), 8.23- 8.05 (m, 2H), 7.93 (d, J = 9.6 Hz, 1H), 7.78- 7.70 (m, 1H), 4.80- 4.69 (m, 2H), 4.67- 4.50 (m, 2H), 4.47- 4.39 (m, 1H), 4.33- 4.22 (m, 2H), 4.09- 4.02 (m, 1H), 2.50 (d, J = 8.8 Hz, 3H), 2.35- |
| | | | | 2.25 (m, 1H), 2.16- |
| | | | | 2.05 (m, 2H), 2.03- |
| | | | | 1.79 (m, 7H) |
| 649 | ((2-(((3S,6S,9aS)-3- (3-(3- methylpyrazin-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 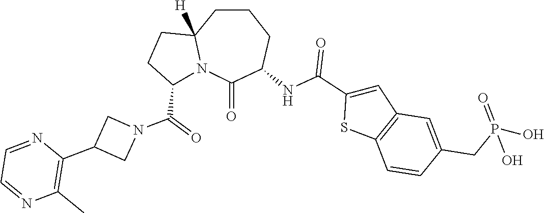 [SMILES: Cc1nccnc1C1CN(C(=O)[C@@H]2CC[C@@H]3CCC[C@H](NC(=O)c4cc5cc(CP(=O)(O)O)ccc5s4)C(=O)N32)C1] | 598.1 | ¹H NMR (400 MHz, CD₃OD) δ 8.50-8.43 (m, 1H), 8.37-8.31 (m, 1H), 7.97 (d, J = 16.8 Hz, 1H), 7.86 (d, J= 12.0 Hz, 1H), 7.76 (d, J = 8.4 Hz, 1H), 7.50-7.44 (m, 1H), 4.89-4.83 (m, 1H), 4.74 (d, J = 10.8 Hz, 1H), 4.60 (d, J = 4.4 Hz, 1H), 4.57-4.53 (m, 1H), 4.46-4.41 |
| | | | | (m, 1H), 4.32 (d, J = |
| | | | | 7.6 Hz, 1H), 4.23 (d, |
| | | | | J = 8.8 Hz, 1H), 4.09- |
| | | | | 3.80 (m, 2H), 3.11 |
| | | | | (d, J = 4.0 Hz, 1H), |
| | | | | 3.06 (d, J = 4.0 Hz, |
| | | | | 1H), 2.50 (d, J = 7.6 |
| | | | | Hz, 3H), 2.29 (d, J = |
| | | | | 6.4 Hz, 1H), 2.11- |
| | | | | 2.02 (m, 2H), 1.97- |
| | | | | 1.81 (m, 6H) |
| 650 | ((2-(((3S,6S,9aS)-3- (3-(5- methylpyrimidin-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 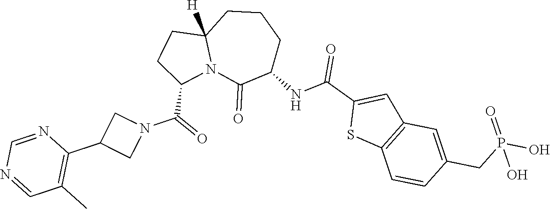 [SMILES: Cc1cncnc1C1CN(C(=O)[C@@H]2CC[C@@H]3CCC[C@H](NC(=O)c4cc5cc(CP(=O)(O)O)ccc5s4)C(=O)N32)C1] | 598.3 | ¹H NMR (400 MHz, CD₃OD) δ 9.02 (d, J = 12.0 Hz, 1H), 8.57- 8.50 (m, 1H), 8.00 (d, J= 19.2 Hz, 1H), 7.87- 7.81 (m, 2H), 7.46- 7.39 (m, 1H), 4.84- 4.81 (m, 1H), 4.76- 4.71 (m, 1H), 4.67- 4.54 (m, 2H), 4.48- 4.39 (m, 1H), 4.34- 4.21 (m, 2H), 4.10- 4.03 (m, 1H), 3.28 (d, |
| | | | | J = 4.4 Hz, 1H), 3.23 |
| | | | | (d, J = 4.4 Hz, 1H), |
| | | | | 2.36-2.29 (m, 1H), |
| | | | | 2.27 (d, J = 6.0 Hz, |
| | | | | 3H), 2.16-2.05 (m, |
| | | | | 2H), 2.02-1.81 (m, |
| | | | | 7H) |
| 651 | ((2-(((3S,6S,10aS)- 3-(3-methoxy-3- (pyridin-3- yl)azetidine-1- carbonyl)-5- oxodecahydropyrrolo [1,2-a]azocin-6- yl)carbamoyl)benzo [b]thiophen-5- yl)methyl) phosphonic acid | 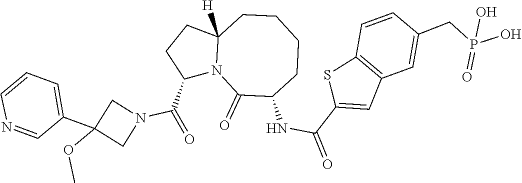 [SMILES: COC1(c2cccnc2)CN(C(=O)[C@@H]2CC[C@@H]3CCCC[C@H](NC(=O)c4cc5cc(CP(=O)(O)O)ccc5s4)C(=O)N32)C1] | 627.0 | ¹H NMR (400 MHz, CD₃OD) δ 8.92-8.84 (m, 1H), 8.76-8.63 (m, 1H), 8.57-8.42 (m, 1H), 8.07-7.96 (m, 1H), 7.92-7.74 (m, 3H), 7.45-7.36 (m, 1H), 4.99 (d, J = 7.6 Hz, 2H), 4.68- 4.53 (m, 1H), 4.52- 4.39 (m, 2H), 4.34- |
| | | | | 4.17 (m, 2H), 3.28- |
| | | | | 3.18 (m, 5H), 2.37- |
| | | | | 2.19 (m, 2H), 2.07- |
| | | | | 1.91 (m, 6H), 1.89- |
| | | | | 1.75 (m, 2H), 1.74- |
| | | | | 1.58 (m,2H) |
| 652 | (difluoro(2- (((3S,6S,9aS)-3-(3- (5-methylpyrimidin- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 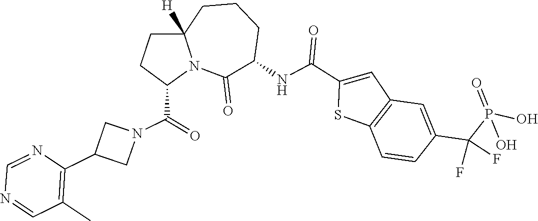 [SMILES: Cc1cncnc1C1CN(C(=O)[C@@H]2CC[C@@H]3CCC[C@H](NC(=O)c4cc5cc(C(F)(F)P(=O)(O)O)ccc5s4)C(=O)N32)C1] | 634.0 | ¹H NMR (400 MHz, CD₃OD) δ 9.04-8.94 (m, 1H), 8.57-8.45 (m, 1H), 8.24-8.15 (m, 1H), 8.13-8.05 (m, 1H), 7.97-7.87 (m, 1H), 7.80-7.71 (m, 1H), 4.89-4.83 (m, 1H), 4.76-4.73 (m, 1H), 4.64-4.54 (m, 2H), 4.49-4.40 (m, 1H), 4.33-4.20 (m, 2H), 4.10-4.01 (m, 1H), 2.31 (d, J = |
| | | | | 4.8 Hz, 1H), 2.29- |
| | | | | 2.24 (m, 3H), 2.16- |
| | | | | 2.05 (m, 2H), 2.03- |
| | | | | 1.80 (m, 7H) |
| 653 | ((2-(((3S,6S,9aS)-3- (3-(6-cyanopyridin- 2-yl)azetidine-1- carbonyl)-5- oxooctahydro-1H- pyrrolo[1,2- a]azepin-6- yl)carbamoyl)benzo [b]thiophen-5- yl)methyl) phosphonic acid | 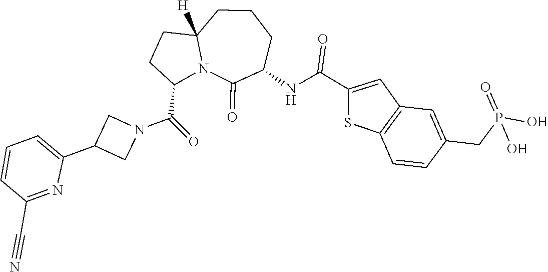 [SMILES: N#Cc1cccc(C2CN(C(=O)[C@@H]3CC[C@@H]4CCC[C@H](NC(=O)c5cc6cc(CP(=O)(O)O)ccc6s5)C(=O)N43)C2)n1] | 608.0 | ¹H NMR (400 MHz, CD₃OD) δ 8.03-7.90 (m, 2H), 7.86 (d, J = 7.2 Hz, 1H), 7.82- 7.57 (m, 3H), 7.51- 7.42 (m, 1H), 4.72- 4.69 (m, 1H), 4.61- 4.58 (m, 1H), 4.44 (s, 3H), 4.14 (d, J = 6.0 Hz, 3H), 3.18-3.09 (m, 2H), 2.35-2.27 (m, 1H), 2.18-2.06 (m, 2H), 2.04 (s, 2H), 2.01-1.94 (m, 2H), 1.93-1.86 (m, 2H), 1.84 (s, 1H) |
| 654 | (difluoro(2- (((3S,6S,10aS)-3-(3- (methoxymethyl)-3- (pyrimidin-2- yl)azetidine-1- carbonyl)-5- oxodecahydropyrrolo [1,2-a]azocin-6- yl)carbamoyl)benzo [b]thiophen-5- yl)methyl) phosphonic acid | 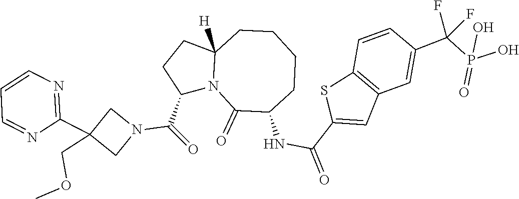 [SMILES: COCC1(c2ncccn2)CN(C(=O)[C@@H]2CC[C@@H]3CCCC[C@H](NC(=O)c4cc5cc(C(F)(F)P(=O)(O)O)ccc5s4)C(=O)N32)C1] | 678.4 | ¹H NMR (400 MHz, ACETONITRILE- d₃) δ 8.07 (s, 2H), 8.00-7.92 (m, 2H), 7.75 (s, 1H), 7.57 (d, J = 8.4 Hz, 1H), 5.71- 5.60 (m, 4H), 5.01 (q, J = 7.6 Hz, 1H), 4.93-4.84 (m, 1H), 4.23 (d, J = 4.8 Hz, 1H), 3.79-3.43 (m, 8H), 2.39-2.30 (m, |
| | | | | 2H), 2.10 (d, J = 7.6 |
| | | | | Hz, 2H), 1.92-1.82 |
| | | | | (m, 2H), 1.67 (m, |
| | | | | 1H), 1.52-1.40 (m, |
| | | | | 1H), 1.31 (s, 1H), |
| | | | | 1.15 (s, 18H), 0.93- |
| | | | | 0.84 (m, 1H), 0.80 |
| | | | | (m, 1H), 0.01 (m, 1H) |
| 655 | ((2- (((3S,6S,9S,10aR)- 9-methyl-3-(3-(4- methylpyridin-3- yl)azetidine-1- carbonyl)-5- oxodecahydropyrrolo [1,2-a]azocin-6- yl)carbamoyl)benzo [b]thiophen-5- yl)methyl) phosphonic acid | 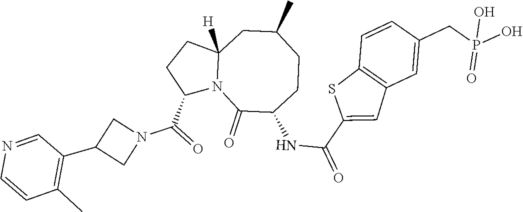 [SMILES: Cc1ccncc1C1CN(C(=O)[C@@H]2CC[C@@H]3C[C@@H](C)CC[C@H](NC(=O)c4cc5cc(CP(=O)(O)O)ccc5s4)C(=O)N32)C1] | 625.0 | ¹H NMR (400 MHz, CD₃OD) δ 8.75 (d, J = 12.0 Hz, 1H), 8.56- 8.36 (m, 1H), 8.02- 7.92 (m, 1H), 7.85- 7.66 (m, 3H), 7.39 (dd, J = 7.6, 17.6 Hz, 1H), 4.84-4.70 (m, 2H), 4.67-4.52 (m, 1H), 4.49-4.37 (m, 3H), 4.32-4.21 (m, 1H), 4.19-4.10 (m, 1H), 3.25 (s, 1H), |
| | | | | 3.19 (s, 1H), 2.52- |
| | | | | 2.40 (m, 3H), 2.37- |
| | | | | 2.27 (m, 1H), 2.23- |
| | | | | 2.06 (m, 3H), 2.00- |
| | | | | 1.91 (m, 2H), 1.90- |
| | | | | 1.81 (m, 2H), 1.80- |
| | | | | 1.68 (m, 2H), 1.42- |
| | | | | 1.32 (m, 1H), 1.05 (d, |
| | | | | J = 6.4 Hz, 3H) |
| 656 | ((2- (((3S,6S,9R,10aR)- 9-methyl-3-(3-(4- methylpyridin-3- yl)azetidine-1- carbonyl)-5- oxodecahydropyrrolo [1,2-a]azocin-6- yl)carbamoyl)benzo [b]thiophen-5- yl)methyl) phosphonic acid | 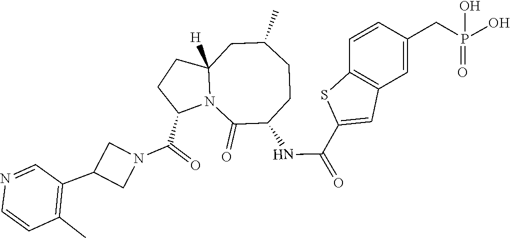 [SMILES: Cc1ccncc1C1CN(C(=O)[C@@H]2CC[C@@H]3C[C@H](C)CC[C@H](NC(=O)c4cc5cc(CP(=O)(O)O)ccc5s4)C(=O)N32)C1] | 625.1 | ¹H NMR (400 MHz, CD₃OD) δ 8.83-8.69 (m, 1H), 8.50-8.36 (m, 1H), 7.99 (s, 1H), 7.85-7.67 (m, 3H), 7.47-7.31 (m, 1H), 4.72 (s, 1H), 4.65- 4.55 (m, 1H), 4.54- 4.53 (m, 1H), 4.51- 4.43 (m, 3H), 4.42- 4.34 (m, 1H), 4.28- 4.25 (m, 1H), 4.13 (dd, J = 6.4, 10.0 Hz, 1H), 3.27-3.15 (m, 2H), 2.47 (s, 3H), |
| | | | | 2.29-2.21 (m, 2H), |
| | | | | 2.09-1.99 (m, 3H), |
| | | | | 1.93-1.79 (m, 3H), |
| | | | | 1.63 (m, 1H), 1.52- |
| | | | | 1.41 (m, 1H), 1.06 (s, |
| | | | | 3H) |
| 657 | ((2-(((3S,6S,9aS)-3- (3-(3- methylpyridin-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 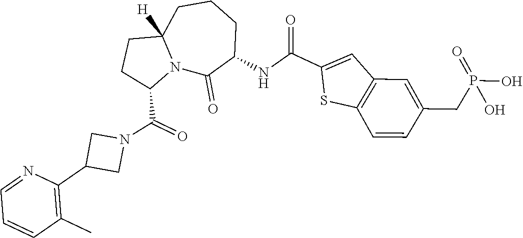 [SMILES: Cc1cccnc1C1CN(C(=O)[C@@H]2CC[C@@H]3CCC[C@H](NC(=O)c4cc5cc(CP(=O)(O)O)ccc5s4)C(=O)N32)C1] | 597.2 | ¹H NMR (400 MHz, CD₃OD) δ 8.46-8.29 (m, 1H), 8.05-7.90 (m, 1H), 7.88-7.74 (m, 2H), 7.68-7.58 (m, 1H), 7.48-7.37 (m, 1H), 7.28-7.17 (m, 1H), 4.85-4.69 (m, 2H), 4.68-4.50 (m, 2H), 4.46-4.16 (m, 3H), 4.06 (d, J = 5.2 Hz, 1H), 3.28- 3.17 (m, 2H), 2.37- 2.22 (m, 4H), 2.20- 1.73 (m, 10H) |
| 658 | (difluoro(2- (((3S,6S,9aS)-3-(3- (3-methylpyridin-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 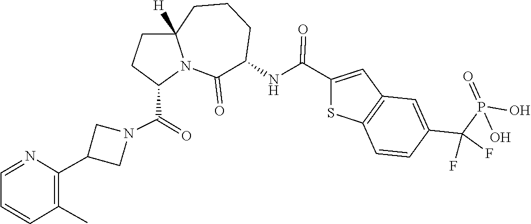 [SMILES: Cc1cccnc1C1CN(C(=O)[C@@H]2CC[C@@H]3CCC[C@H](NC(=O)c4cc5cc(C(F)(F)P(=O)(O)O)ccc5s4)C(=O)N32)C1] | 633.0 | ¹H NMR (400 MHz, DMSO-d₆) δ 8.78 (dd, J = 7.2, 12.8 Hz, 1H), 8.42 (dd, J = 4.0, 14.0 Hz, 1H), 8.30 (d, J = 19.6 Hz, 1H), 8.19- 7.99 (m, 2H), 7.70- 7.53 (m, 2H), 7.29- 7.13 (m, 1H), 4.73- 4.41 (m, 4H), 4.30- 4.06 (m, 4H), 2.21 (d, J = 3.0 Hz, 3H), 2.09- 1.60 (m, 10H) |
| 659 | ((2-(((3S,6S,9aS)-3- (3-(5-cyanopyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 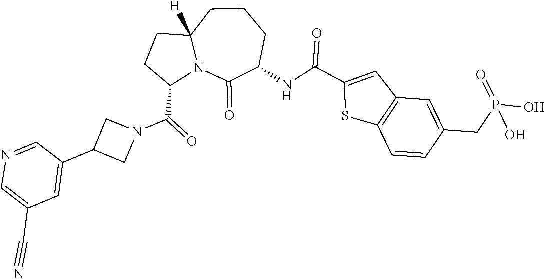 [SMILES: N#Cc1cncc(C2CN(C(=O)[C@@H]3CC[C@@H]4CCC[C@H](NC(=O)c5cc6cc(CP(=O)(O)O)ccc6s5)C(=O)N43)C2)c1] | 607.9 | ¹H NMR (400 MHz, DMSO-d₆) δ 8.93 (s, 1H), 8.86 (dd, J = 2.0, 10.4 Hz, 1H), 8.70 (dd, J = 7.6, 10.4 Hz, 1H), 8.50-8.34 (m, 1H), 8.17 (d, J = 12.8 Hz, 1H), 7.90 (dd, J = 5.6, 8.0 Hz, 1H), 7.79 (s, 1H), 7.36 (dd, J = 1.2, 8.4 Hz, 1H), 4.82- 4.59 (m, 2H), 4.48- 4.24 (m, 3H), 4.06- 3.90 (m, 3H), 3.15- 3.04 (m, 2H), 2.25- 2.15 (m, 1H), 2.09- 1.93 (m, 2H), 1.92- |
| | | | | 1.77 (m, 4H), 1.73 (s, |
| | | | | 2H), 1.70-1.60 (m, |
| | | | | 1H) |
| 660 | ((2-(((3S,6S,9aS)-3- (3-(4- cyanopyrimidin-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 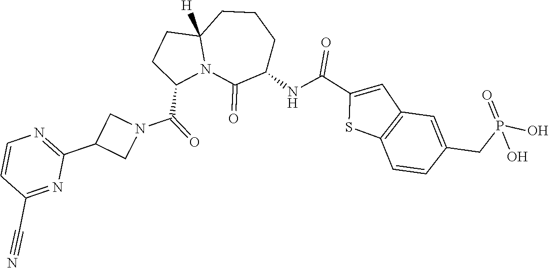 [SMILES: N#Cc1ccnc(C2CN(C(=O)[C@@H]3CC[C@@H]4CCC[C@H](NC(=O)c5cc6cc(CP(=O)(O)O)ccc6s5)C(=O)N43)C2)n1] | 609.0 | ¹H NMR (400 MHz, DMSO-d₆) δ 9.16 (m, 1H) 8.69 (m, 1H) 8.16 (d, J=16.0 Hz, 1H) 8.04 (m, 1H) 7.90 (m, 1H) 7.78 (m, 1H) 7.36 (m, 1H) 4.77 (m, 1H) 4.56- 4.65 (m, 2H) 4.40- 4.49 (m, 2H) 4.21- 4.30 (m, 2H) 4.11- 4.17 (m, 1H) 3.98- 4.05 (m, 2H) 3.06- 3.14 (m, 2H) 2.16- 2.24 (m, 1H) 1.95- 2.06 (m, 2H) 1.69- 1.87 (m, 7H) |
| 661 | ((2-(((3S,6S,9aS)-3- (3-(4- methylpyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 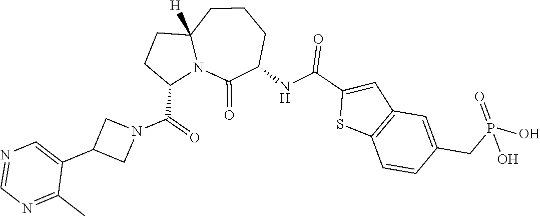 [SMILES: Cc1ncncc1C1CN(C(=O)[C@@H]2CC[C@@H]3CCC[C@H](NC(=O)c4cc5cc(CP(=O)(O)O)ccc5s4)C(=O)N32)C1] | 597.9 | ¹H NMR (400 MHz, CD₃OD) δ 8.96-8.90 (m, 1H), 8.89-8.73 (m, 1H), 8.03-7.95 (m, 1H), 7.87-7.79 (m, 2H), 7.47-7.38 (m, 1H), 4.95-4.90 (m, 1H), 4.76-4.64 (m, 2H), 4.60-4.53 (m, 1H), 4.51-4.37 (m, 1H), 4.24-4.11 (m, 2H), 4.10-4.02 (m, 1H), 3.29-3.20 |
| | | | | (m, 2H), 2.52-2.45 |
| | | | | (m, 3H), 2.35-2.25 |
| | | | | (m, 1H), 2.19-2.04 |
| | | | | (m, 2H), 2.03-1.72 |
| | | | | (m, 7H) |
| 662 | ((2-(((3S,6S,10aS)- 3-(3-(1-methyl-1H- benzo[d]imidazol-2- yl)azetidine-1- carbonyl)-5- oxodecahydropyrrolo [1,2-a]azocin-6- yl)carbamoyl)benzo [b]thiophen-5- yl)methyl) phosphonic acid | 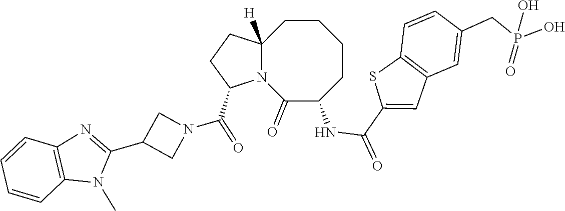 [SMILES: Cn1c(C2CN(C(=O)[C@@H]3CC[C@@H]4CCCC[C@H](NC(=O)c5cc6cc(CP(=O)(O)O)ccc6s5)C(=O)N43)C2)nc2ccccc21] | 650.2 | ¹H NMR (400 MHz, DMSO-d₆) δ 8.85- 8.70 (m, 1H), 8.26- 8.07 (m, 1H), 7.84- 7.64 (m, 2H), 7.63- 7.58 (m, 1H), 7.51- 7.46 (m, 1H), 7.30 (d, J = 6.0 Hz, 1H), 7.25- 7.11 (m, 2H), 4.92 (d, J = 7.6 Hz, 1H), 4.79-4.47 (m, 2H), 4.39-4.13 (m, 5H), 3.66 (d, J = 1.6 Hz, |
| | | | | 3H), 2.92-2.79 (m, |
| | | | | 2H), 2.35-1.41 (m, |
| | | | | 12H) |
| 663 | ((2-(((3S,6S,10aS)- 5-oxo-3-(3-(pyridin- 3-yl)azetidine-1- carbonyl)decahydro pyrrolo[1,2- a]azocin-6- yl)carbamoyl)benzo [b]thiophen-5- yl)methyl) phosphonic acid | 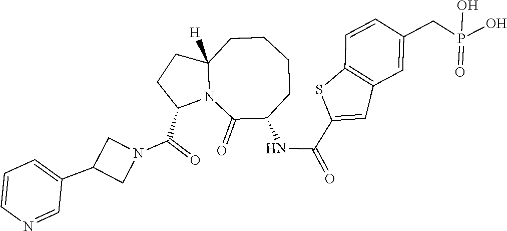 [SMILES: O=C(N[C@H]1CCCC[C@H]2CC[C@@H](C(=O)N3CC(c4cccnc4)C3)N2C1=O)c1cc2cc(CP(=O)(O)O)ccc2s1] | 597.2 | ¹H NMR (400 MHz, CD₃OD) δ 8.83-8.68 (m, 1H), 8.66-8.58 (m, 1H), 8.55-8.37 (m, 1H), 8.04 (s, 1H), 7.91-7.73 (m, 3H), 7.42 (d, J = 8.4 Hz, 1H), 5.04-5.00 (m, 1H), 4.73 (s, 1H), 4.65 (d, J = 5.6 Hz, 1H), 4.56-4.43 (m, 3H), 4.15-4.01 (m, 2H), 3.28-3.20 (m, 2H), 2.30-2.26 (m, 1H), 2.07-2.01 (m, |
| | | | | 3H), 1.88-1.80 (m, |
| | | | | 2H), 1.72-1.61 (m, |
| | | | | 2H), 1.42-1.24 (m, |
| | | | | 4H) |
| 664 | (fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 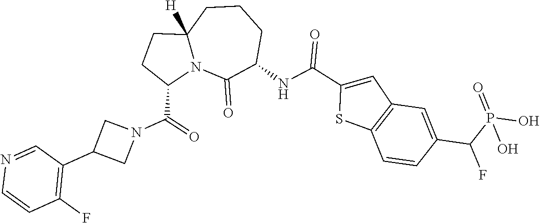 [SMILES: O=C(N[C@H]1CCC[C@H]2CC[C@@H](C(=O)N3CC(c4cnccc4F)C3)N2C1=O)c1cc2cc(C(F)P(=O)(O)O)ccc2s1] | 619.0 | ¹H NMR (400 MHz, DMSO-d₆) δ 8.80- 8.68 (m, 1H), 8.60- 8.31 (m, 1H), 8.29- 8.14 (m, 1H), 8.09- 7.94 (m, 2H), 7.59- 7.46 (m, 1H), 7.41- 7.01 (m, 1H), 5.92- 5.75 (m, 1H), 4.69- 4.63 (m, 1H), 4.59- 4.47 (m, 1H), 4.45- 4.42 (m, 1H), 4.32 4.25 (m, 1H), 4.22- 4.11 (m, 1H), 4.09- |
| | | | | 3.96 (m, 3H), 2.26- |
| | | | | 2.12 (m, 1H), 2.11- |
| | | | | 1.92 (m, 2H), 1.92- |
| | | | | 1.75 (m, 4H), 1.73 (s, |
| | | | | 3H) |
| 665 | ((R)-(2- (((3S,6S,9aS)-3-(3- (4-(dimethylamino) pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 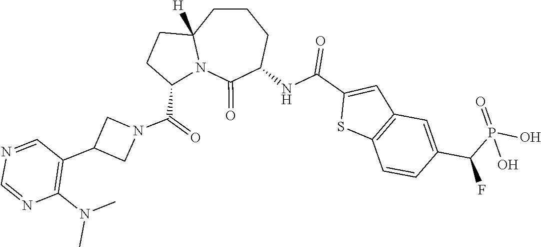 [SMILES: CN(C)c1ncncc1C1CN(C(=O)[C@@H]2CC[C@@H]3CCC[C@H](NC(=O)c4cc5cc([C@H](F)P(=O)(O)O)ccc5s4)C(=O)N32)C1] | 645.2 | ¹H NMR (400 MHz, CD₃OD) δ 8.35-8.24 (m, 1H), 8.20-8.12 (m, 1H), 8.07-7.97 (m, 1H), 7.93 (d, J = 9.6 Hz, 1H), 7.86 (d, J = 8.4 Hz, 1H), 7.64- 7.54 (m, 1H), 5.84- 5.66 (m, 1H), 4.77- 4.61 (m, 2H), 4.56- 4.44 (m, 2H), 4.36 (s, 1H), 4.25-4.14 (m, 1H), 4.12-3.94 (m, 2H), 3.30 (s, 2H), 3.24 (s, 4H), 2.34- |
| | | | | 2.26 (m, 1H), 2.20- |
| | | | | 2.06 (m, 2H), 2.06- |
| | | | | 1.96 (m, 2H), 1.96- |
| | | | | 1.89 (m, 2H), 1.89- |
| | | | | 1.74 (m, 3H) |
| 666 | ((2-(((3S,6S,9aS)-3- (3-(4-((S)-3- fluoropyrrolidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 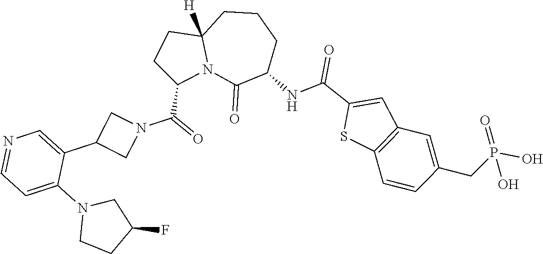 [SMILES: O=C(N[C@H]1CCC[C@H]2CC[C@@H](C(=O)N3CC(c4cnccc4N4CC[C@H](F)C4)C3)N2C1=O)c1cc2cc(CP(=O)(O)O)ccc2s1] | 670.3 | ¹H NMR (400 MHz, CD₃OD) δ 8.36 (s, 1H), 8.08-7.93 (m, 2H), 7.87-7.79 (m, 2H), 7.47-7.40 (m, 1H), 6.92 (s, 1H), 5.50-5.33 (m, 1H), 4.81-4.61 (m, 2H), 4.61-4.49 (m, 2H), 4.48-4.45 (m, 1H), 4.45-4.39 (m, 1H), 4.21-4.06 (m, 2H), 4.0 (s, 2H), 3.80 (s, 2H), 3.30-3.20 (m, 2H), 2.46-2.37 (m, 1H), 2.36-2.30 (m, |
| | | | | 1H), 2.28-2.08 (m, |
| | | | | 3H), 2.08-1.93 (m, |
| | | | | 4H), 1.92 (s, 3H) |
| 667 | ((S)-(2- (((3S,6S,9aS)-3-(3- (4- (dimethylamino) pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 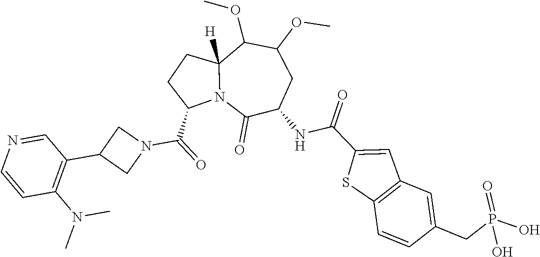 [SMILES: COC1C[C@H](NC(=O)c2cc3cc(CP(=O)(O)O)ccc3s2)C(=O)N2[C@H](CC[C@H]2C(=O)N2CC(c3cnccc3N(C)C)C2)C1OC] | 645.2 | ¹H NMR (400 MHz, CD₃OD) δ 8.43 (d, J = 8.4 Hz, 1H), 8.32- 8.19 (m, 1H), 8.07- 7.96 (m, 2H), 7.93- 7.86 (m, 1H), 7.66- 7.55 (m, 1H), 5.88- 5.65 (m, 1H), 4.73 (d, J = 11.2 Hz, 1H), 4.66 (t, J = 8.8 Hz, 1H), 4.57-4.48 (m, 2H), 4.42-4.33 (m, 1H), 4.29-4.22 (m, 1H), 4.12-3.99 (m, 2H), 3.28 (d, J = 3.6 Hz, 6H), 2.36-2.25 (m, |
| | | | | 1H), 2.16-2.06 (m, |
| | | | | 2H), 2.05-1.94 (m, |
| | | | | 3H), 1.94-1.86 (m, |
| | | | | 2H), 1.85 (s, 2H) |
| 668 | ((2-(((3S,6S,9aS)-3- (3-(4-((R)-2- (difluoromethyl) azetidin-1-yl) pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 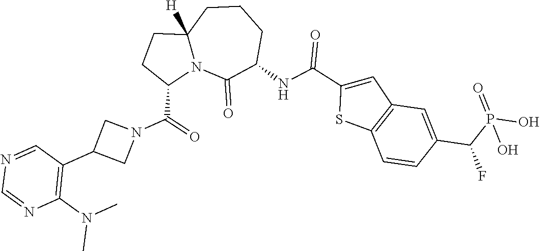 [SMILES: CN(C)c1ncncc1C1CN(C(=O)[C@@H]2CC[C@@H]3CCC[C@H](NC(=O)c4cc5cc([C@@H](F)P(=O)(O)O)ccc5s4)C(=O)N32)C1] | 688.2 | ¹H NMR (400 MHz, CD₃OD) δ 8.50-8.34 (m, 1H), 8.20-7.94 (m, 2H), 7.89-7.75 (m, 2H), 7.52-7.33 (m, 1H), 7.02-6.82 (m, 1H), 6.43-6.03 (m, 1H), 4.69-4.64 (m, 2H), 4.56-4.49 (m, 2H), 4.42-4.33 (m, 2H), 4.28-4.26 (m, 1H), 4.16-4.06 (m, 3H), 4.01-3.97 (m, 1H), 3.29-3.26 (m, 2H), 2.71-2.64 |
| | | | | (m, 1H), 2.47-2.42 |
| | | | | (m, 1H), 2.33-2.28 |
| | | | | (m, 1H), 2.12-2.06 |
| | | | | (m, 2H), 1.99-1.93 |
| | | | | (m, 3H), 1.89-1.78 |
| | | | | (m, 4H) |
| 669 | ((S)-fluoro(2- (((3S,6S,9aS)-3-(3- (4-((S)-2- (methoxymethyl) azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 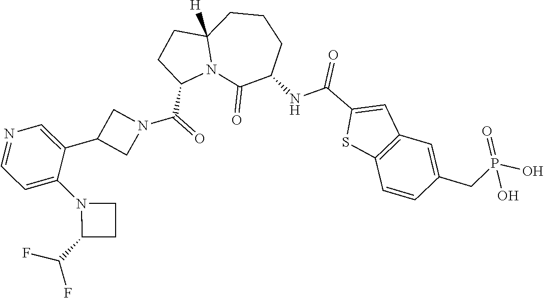 [SMILES: O=C(N[C@H]1CCC[C@H]2CC[C@@H](C(=O)N3CC(c4cnccc4N4CC[C@@H]4C(F)F)C3)N2C1=O)c1cc2cc(CP(=O)(O)O)ccc2s1] | 701.5 | ¹H NMR (400 MHz, DMSO-d₆) δ 8.72 (dd, J = 7.6, 16.4 Hz, 1H), 8.62 (d, J = 18.2 Hz, 1H), 8.43-8.27 (m, 1H), 8.23 (d, J = 12.0 Hz, 1H), 8.07- 8.01 (m, 1H), 7.98 (s, 1H), 7.53 (d, J = 8.8 Hz, 1H), 5.96-5.71 (m, 1H), 4.81-4.72 (m, 1H), 4.65 (t, J = 8.8 Hz, 2H), 4.53- 4.40 (m, 3H), 4.26- 4.13 (m, 3H), 4.00- 3.93 (m, 3H), 3.76 (d, J = 5.2 Hz, 1H), 3.59 (d, J = 7.6 Hz, 1H), |
| | | | | 3.29 (d, J = 3.6 Hz, |
| | | | | 3H), 2.45-2.37 (m, |
| | | | | 1H), 2.33-2.16 (m, |
| | | | | 2H), 2.07-1.92 (m, |
| | | | | 2H), 1.90-1.62 (m, |
| | | | | 7H) |
| 670 | (fluoro(2- (((3S,6S,9aS)-3-(3- (2-morpholinophenyl) azetidine-1- carbonyl)-5- oxooctahydro-1H- pyrrolo[1,2- a]azepin-6- yl)carbamoyl)benzo [b]thiophen-5- yl)methyl) phosphonic acid | 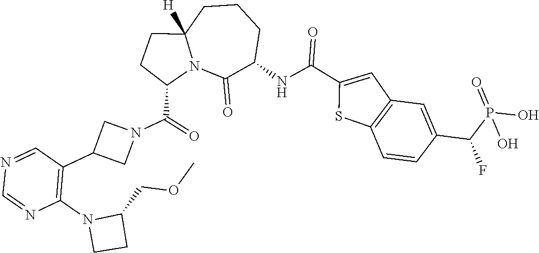 [SMILES: COC[C@@H]1CCN1c1ncncc1C1CN(C(=O)[C@@H]2CC[C@@H]3CCC[C@H](NC(=O)c4cc5cc([C@@H](F)P(=O)(O)O)ccc5s4)C(=O)N32)C1] | 685.1 | ¹H NMR (400 MHz, DMSO-d₆) δ 8.65- 8.41 (m, 1H), 8.21 (d, J = 7.2 Hz, 1H), 8.10- 7.91 (m, 3H), 7.71- 7.51 (m, 3H), 5.92- 5.71 (m, 1H), 4.73- 4.51 (m, 2H), 4.42- 4.25 (m, 1H), 4.24- 4.11 (m, 2H), 4.11- 4.03 (m, 2H), 4.03- 3.92 (m, 2H), 3.91- 3.83 (m, 1H), 3.75- 3.65 (m, 2H), 3.63- 3.56 (m, 2H), 3.53- |
| | | | | 3.47 (m, 2H), 2.23- |
| | | | | 2.10 (m, 1H), 2.08- |
| | | | | 1.87 (m, 3H), 1.87- |
| | | | | 1.53 (m, 6H) |
| 671 | ((2-(((3S,6S,9aS)-3- (3-(4-(3-oxa-6- azabicyclo[3.1.1] heptan-6-yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 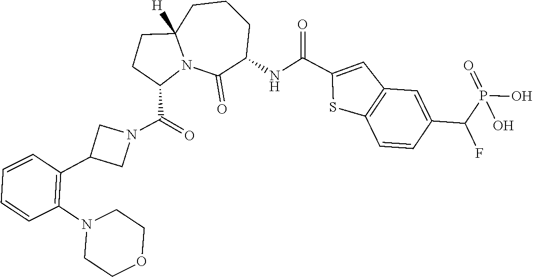 [SMILES: O=C(N[C@H]1CCC[C@H]2CC[C@@H](C(=O)N3CC(c4ccccc4N4CCOCC4)C3)N2C1=O)c1cc2cc(C(F)P(=O)(O)O)ccc2s1] | 698.2 | ¹H NMR (400 MHz, DMSO-d₆) δ 8.82- 8.54 (m, 1H), 8.23- 8.07 (m, 2H), 7.95- 7.84 (m, 2H), 7.54- 7.42 (m, 1H), 6.71- 6.51 (m, 1H), 5.56- 5.44 (m, 1H), 4.68- 4.61 (m, 1H), 4.53- 4.38 (m, 4H), 4.28- 4.20 (m, 1H), 4.18- 4.10 (m, 1H), 4.03- 3.90 (m, 5H), 3.70 (d, J = 6.2 Hz, 2H), 2.23- 2.17 (m, 1H), 2.02- 1.94 (m, 2H), 1.88- 1.63 (m, 9H) |
| 672 | ((2-(((3S,6S,9aS)-3- (3-(5,6-dihydro-2H- pyran-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 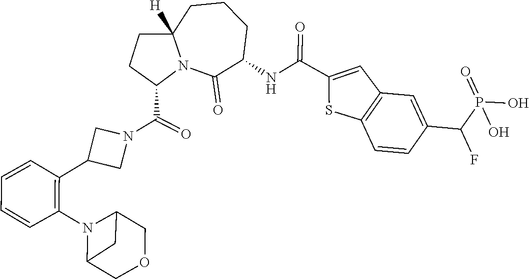 [SMILES: O=C(N[C@H]1CCC[C@H]2CC[C@@H](C(=O)N3CC(c4ccccc4N4C5COCC4C5)C3)N2C1=O)c1cc2cc(C(F)P(=O)(O)O)ccc2s1] | 588.2 | ¹H NMR (400 MHz, CD₃OD) δ 8.01 (s, 1H), 7.85 (d, J = 4.8 Hz, 2H), 7.43 (d, J = 8.4 Hz, 1H), 5.83 (s, 1H), 4.73 (d, J = 10.8 Hz, 1H), 4.64-4.49 (m, 2H), 4.44-4.32 (m, 1H), 4.20-4.13 (m, 1H), 4.07 (t, J = 9.6 Hz, 4H), 3.96- 3.84 (m, 1H), 3.74 (td, J = 5.6, 8.0 Hz, 2H), 3.28 (s, 1H), 3.24-3.21 (m, 1H), 2.34-2.25 (m, 1H), 2.17 (td, J = 1.6, 2.8 |
| | | | | Hz, 2H), 2.12-2.04 |
| | | | | (m, 2H), 2.01 (d, J = |
| | | | | 12.0 Hz, 1H), 1.97- |
| | | | | 1.86 (m, 4H), 1.83 (d, |
| | | | | J = 11.2 Hz, 2H) |
| 673 | (fluoro(2- (((3S,6S,9aS)-3- ((2R,3R)-2-(5- methyl-1,3,4- oxadiazol-2-yl)-3- phenylazetidine-1- carbonyl)-5- oxooctahydro-1H- pyrrolo[1,2- a]azepin-6- yl)carbamoyl)benzo [b]thiophen-5- yl)methyl) phosphonic acid | 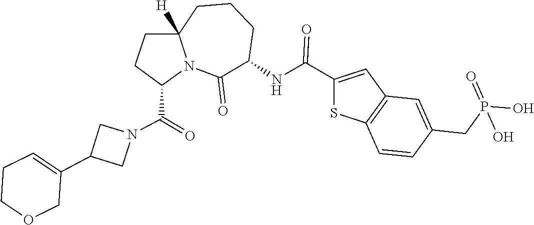 [SMILES: O=C(N[C@H]1CCC[C@H]2CC[C@@H](C(=O)N3CC(C4=CCCOC4)C3)N2C1=O)c1cc2cc(CP(=O)(O)O)ccc2s1] | 682.4 | ¹H NMR (400 MHz, CD₃OD) δ 8.12-8.05 (m, 2H), 7.95 (d, J = 8.4 Hz, 1H), 7.61 (d, J = 8.0 Hz, 1H), 7.53 (d, J = 7.6 Hz, 1H), 7.41 (d, J = 4.0 Hz, 1H), 7.39-7.34 (m, 3H), 5.92-5.76 (m, 1H), 5.46-5.45 (m, 1H), 4.81-4.61 (m, 3H), 4.48-4.40 (m, 1H), 4.17-4.04 (m, 2H), 2.58-2.48 (m, |
| | Or | | | 3H), 2.35-2.14 (m, |
| | (fluoro(2- | HPLC Peak 1 of LHS BB used | | 2H), 2.12-2.06 (m, |
| | (((3S,6S,9aS)-3- | for synthesis and biological | | 1H), 2.01-2.00 (m, |
| | ((2S,3S)-2-(5- | testing | | 1H), 1.95-1.79 (m, |
| | methyl-1,3,4- | | | 6H) |
| | oxadiazol-2-yl)-3- | | | |
| | phenylazetidine-1- | | | |
| | carbonyl)-5- | | | |
| | oxooctahydro-1H- | | | |
| | pyrrolo[1,2- | | | |
| | a]azepin-6- | | | |
| | yl)carbamoyl)benzo | | | |
| | [b]thiophen-5- | | | |
| | yl)methyl) | | | |
| | phosphonic acid | | | |
| 674 | (fluoro(2- (((3S,6S,9aS)-3- ((2R,3R)-2-(5- methyl-1,3,4- oxadiazol-2-yl)-3- phenylazetidine-1- carbonyl)-5- oxooctahydro-1H- pyrrolo[1,2- a]azepin-6- yl)carbamoyl)benzo [b]thiophen-5- yl)methyl) phosphonic acid Or (fluoro(2- | 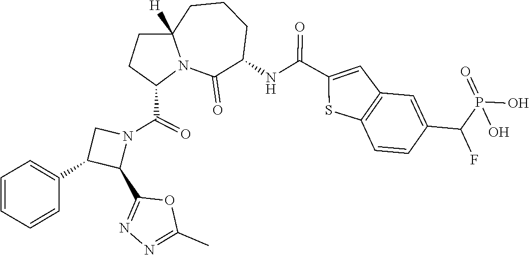 [SMILES: Cc1nnc([C@H]2[C@H](c3ccccc3)CN2C(=O)[C@@H]2CC[C@@H]3CCC[C@H](NC(=O)c4cc5cc(C(F)P(=O)(O)O)ccc5s4)C(=O)N32)o1] | 682.5 | ¹H NMR (400 MHz, CD₃OD) δ 8.10-8.04 (m, 2H), 8.00-7.94 (m, 1H), 7.61-7.59 (m, 1H), 7.49 (d, J = 7.6 Hz, 2H), 7.37- 7.29 (m, 3H), 5.90- 5.76 (m, 1H), 5.40 (d, J = 6.4 Hz, 1H), 4.85- 4.72 (m, 3H), 4.66- 4.64 (m, 1H), 4.17- 4.01 (m, 2H), 2.57- 2.56 (m, 3H), 2.33- 2.28 (m, 1H), 2.22- 2.19 (m, 1H), 2.17- 2.05 (m, 1H), 2.02- |
| | (((3S,6S,9aS)-3- | | | 1.99 (m, 1H), 1.87- |
| | ((2S,3S)-2-(5- | or | | 1.86 (m, 6H) |
| | methyl-1,3,4- | | | |
| | oxadiazol-2-yl)-3- phenylazetidine-1- carbonyl)-5- oxooctahydro-1H- pyrrolo[1,2- a]azepin-6- yl)carbamoyl)benzo [b]thiophen-5- yl)methyl) phosphonic acid | 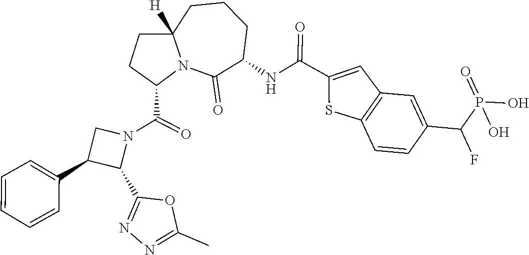 [SMILES: Cc1nnc([C@@H]2[C@@H](c3ccccc3)CN2C(=O)[C@@H]2CC[C@@H]3CCC[C@H](NC(=O)c4cc5cc(C(F)P(=O)(O)O)ccc5s4)C(=O)N32)o1] | | |
| | | HPLC Peak 2 of LHS BB used | | |
| | | for synthesis and biological | | |
| | | testing | | |
| 675 | ((2-(((3S,6S,9aS)-3- (3-(4-((S)-2- (methoxymethyl) azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 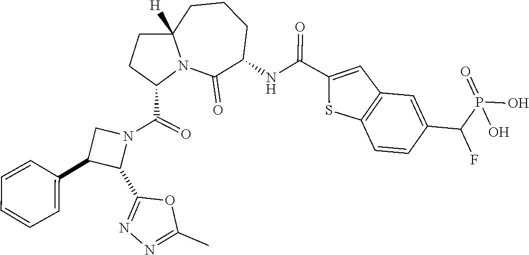 [SMILES: Cc1nnc([C@@H]2[C@@H](c3ccccc3)CN2C(=O)[C@@H]2CC[C@@H]3CCC[C@H](NC(=O)c4cc5cc(C(F)P(=O)(O)O)ccc5s4)C(=O)N32)o1] | 683.2 | ¹H NMR (400 MHz, CD₃OD) δ 8.30-8.17 (m, 1H), 8.04 (d, J = 14.4 Hz, 1H), 7.94 (d, J = 4.0 Hz, 1H), 7.78 (d, J = 5.2 Hz, 1H), 7.72 (d, J = 8.4 Hz, 1H), 7.47-7.39 (m, 1H), 4.72 (t, J = 8.8 Hz, 2H), 4.65-4.57 (m, 1H), 4.57-4.48 (m, 2H), 4.42-4.26 (m, 1H), 4.25-4.16 (m, 1H), 4.09-4.03 (m, 2H), 4.02-3.88 (m, 2H), 3.86-3.83 |
| | | | | (m, 1H), 3.60-3.54 |
| | | | | (m, 1H), 3.36 (d, J = |
| | | | | 12.0 Hz, 3H), 3.17- |
| | | | | 3.03 (m, 2H), 2.56- |
| | | | | 2.42 (m, 1H), 2.42- |
| | | | | 2.24 (m, 2H), 2.16- |
| | | | | 2.04 (m, 2H), 2.01- |
| | | | | 1.78 (m, 7H) |
| 676 | ((R)-(2- (((3S,6S,8R,9aR)-3- (3-(4-(azetidin-1- yl)pyrimidin-5- yl)azetidine-1- carbonyl)-8-methyl- 5-oxooctahydro-1H- pyrrolo[1,2- a]azepin-6- yl)carbamoyl)benzo [b]thiophen-5- yl)fluoromethyl) phosphonic acid | 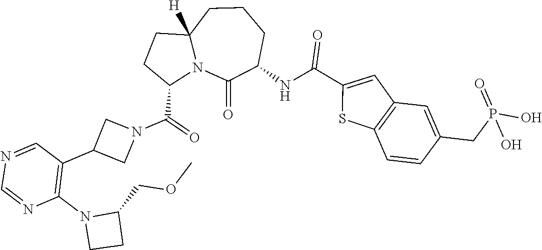 [SMILES: COC[C@@H]1CCN1c1ncncc1C1CN(C(=O)[C@@H]2CC[C@@H]3CCC[C@H](NC(=O)c4cc5cc(CP(=O)(O)O)ccc5s4)C(=O)N32)C1] | 671.4 | ¹H NMR (400 MHz, CD₃OD) δ 8.36-8.02 (m, 2H), 7.99 (d, J = 6.8 Hz, 2H), 7.87 (t, J = 10.0 Hz, 1H), 7.56 (t, J = 8.4 Hz, 1H), 5.83-5.69 (m, 1H), 4.75 (d, J = 11.2 Hz, 2H), 4.55-4.43 (m, 6H), 4.29-4.14 (m, 1H), 4.08-4.04 (m, 3H), 2.47-2.43 (m, 2H), 2.35-2.25 (m, 1H), 2.15-2.03 (m, 2H), 1.97-1.91 (m, |
| | | | | 3H), 1.80 (d, J = 13.6 |
| | | | | Hz, 1H), 1.63-1.60 |
| | | | | (m, 2H), 1.05 (d, J = |
| | | | | 4.0 Hz, 3H) |
| 677 | ((S)-(2- (((3S,6S,8R,9aR)-3- (3-(4-(azetidin-1- yl)pyrimidin-5- yl)azetidine-1- carbonyl)-8-methyl- 5-oxooctahydro-1H- pyrrolo[1,2- a]azepin-6- yl)carbamoyl)benzo [b]thiophen-5- yl)fluoromethyl) phosphonic acid | 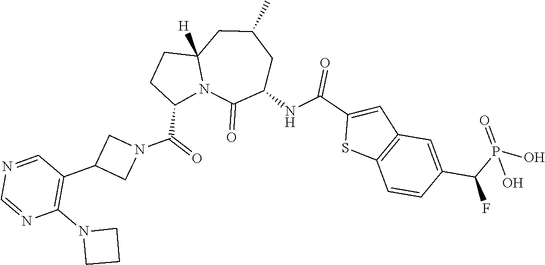 [SMILES: C[C@H]1C[C@H]2CC[C@@H](C(=O)N3CC(c4cncnc4N4CCC4)C3)N2C(=O)[C@@H](NC(=O)c2cc3cc([C@H](F)P(=O)(O)O)ccc3s2)C1] | 671.4 | ¹H NMR (400 MHz, CD₃OD) δ 8.40-8.18 (m, 1H), 8.08-7.94 (m, 3H), 7.89 (d, J = 8.4 Hz, 1H), 7.61 (t, J = 9.2 Hz, 1H), 5.85- 5.68 (m, 1H), 4.79- 4.71 (m, 2H), 4.61- 4.42 (m, 6H), 4.31- 4.19 (m, 1H), 4.14- 3.97 (m, 3H), 2.53- 2.41 (m, 2H), 2.35- 2.27 (m, 1H), 2.14- 2.02 (m, 2H), 1.99- 1.94 (m, 2H), 1.83- 1.76 (m, 1H), 1.68- |
| | | | | 1.53 (m, 2H), 1.34- |
| | | | | 1.27 (m, 1H), 1.08- |
| | | | | 1.03 (m, 3H) |
| 678 | ((2- (((3S,6S,8R,9aR)-3- (3-(4-(azetidin-1- yl)pyrimidin-5- yl)azetidine-1- carbonyl)-8-methyl- 5-oxooctahydro-1H- pyrrolo[1,2- a]azepin-6- yl)carbamoyl)benzo [b]thiophen-5- yl)methyl) phosphonic acid | 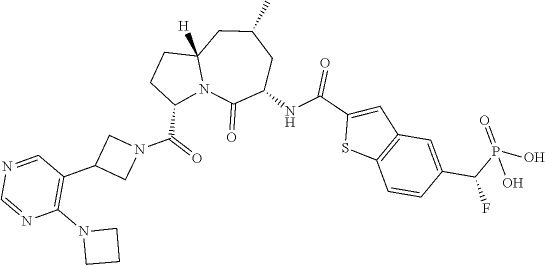 [SMILES: C[C@H]1C[C@H]2CC[C@@H](C(=O)N3CC(c4cncnc4N4CCC4)C3)N2C(=O)[C@@H](NC(=O)c2cc3cc([C@@H](F)P(=O)(O)O)ccc3s2)C1] | 653.1 | ¹H NMR (400 MHz, CD₃OD) δ 8.47 (d, J = 2.0 Hz, 1H), 8.22 (d, J = 17.6 Hz, 1H), 7.99 (d, J = 6.4 Hz, 1H), 7.86-7.81 (m, 2H), 7.45-7.40 (m, 1H), 4.78-4.72 (m, 1H), 4.62-4.49 (m, 6H), 4.45-4.34 (m, 1H), 4.30 (t, J = 8.4 Hz, 1H), 4.14-4.02 (m, 3H), 3.27-3.19 (m, 2H), 2.55-2.43 (m, 2H), 2.32 (td, J = 6.8, 13.2 Hz, 1H), |
| | | | | 2.16-2.03 (m, 2H), |
| | | | | 1.99-1.93 (m, 2H), |
| | | | | 1.80 (dd, J = 2.8, 14.8 |
| | | | | Hz, 1H), 1.67-1.52 |
| | | | | (m, 2H), 1.37-1.27 |
| | | | | (m, 1H), 1.05 (d, J = |
| | | | | 6.8 Hz, 3H) |
| 679 | ((2-(((3S,6S,9aS)-3- (3-(4-((S)-3- methylmorpholino) pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 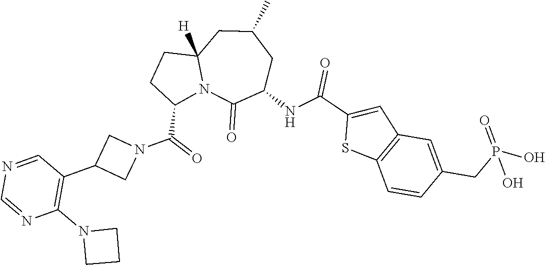 [SMILES: C[C@H]1C[C@H]2CC[C@@H](C(=O)N3CC(c4cncnc4N4CCC4)C3)N2C(=O)[C@@H](NC(=O)c2cc3cc(CP(=O)(O)O)ccc3s2)C1] | 682.2 | ¹H NMR (400 MHz, CD₃OD) δ 8.38-8.10 (m, 1H), 8.08-7.84 (m, 1H), 7.83-7.51 (m, 3H), 7.50-7.25 (m, 1H), 7.15-6.70 (m, 1H), 4.79-4.66 (m, 3H), 4.57-4.28 (m, 3H), 4.25-3.98 (m, 3H), 3.96-3.46 (m, 4H), 3.23-2.94 (m, 3H), 2.92-2.52 (m, 1H), 2.40-2.23 (m, 1H), 2.20-2.06 (m, 2H), 2.05-1.73 (m, 7H), 1.24-0.94 (m, 3H) |
| 680 | ((2-(((3S,6S,9aS)-3- (3-(4-(3- cyanoazetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 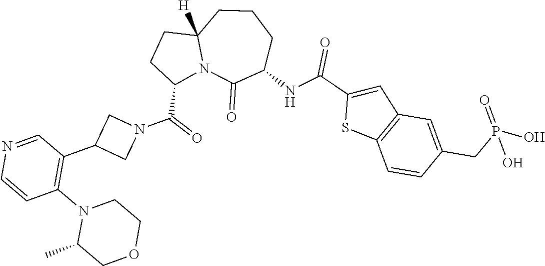 [SMILES: C[C@H]1COCCN1c1ccncc1C1CN(C(=O)[C@@H]2CC[C@@H]3CCC[C@H](NC(=O)c4cc5cc(CP(=O)(O)O)ccc5s4)C(=O)N32)C1] | 663.3 | ¹H NMR (400 MHz, CD₃OD) 8.23 (d, J = 6.8 Hz, 1H), 8.02- 7.88 (m, 2H), 7.83- 7.72 (m, 2H), 7.40 (t, J = 7.2 Hz, 1H), 6.48- 6.42 (m, 1H), 4.85- 4.68 (m, 2H), 4.67- 4.56 (m, 3H), 4.56- 4.40 (m, 3H), 4.39- 4.24 (m, 1H), 4.18- 3.96 (m, 3H), 3.94- 3.80 (m, 1H), 3.29- 3.15 (m, 2H), 2.39- 2.22 (m, 1H), 2.20- 2.05 (m, 2H), 2.05- |
| | | | | 2.01 (m, 1H), 2.01- |
| | | | | 1.89 (m, 3H), 1.89- |
| | | | | 1.72 (m, 3H) |
| 681 | ((2-(((3S,6S,9aS)-3- (3-(4-(azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 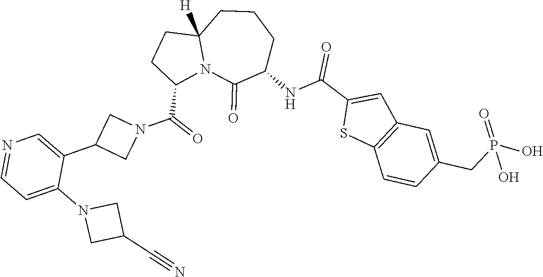 [SMILES: N#CC1CN(c2ccncc2C2CN(C(=O)[C@@H]3CC[C@@H]4CCC[C@H](NC(=O)c5cc6cc(CP(=O)(O)O)ccc6s5)C(=O)N43)C2)C1] | 639.3 | ¹H NMR (400 MHz, CD₃OD) δ 8.46-8.42 (m, 1H), 8.20 (d, J = 12.8 Hz, 1H), 7.98 (d, J = 4.8 Hz, 1H), 7.86- 7.79 (m, 2H), 7.45- 7.39 (m, 1H), 4.73 (d, J = 10.8 Hz, 1H), 4.63- 4.46 (m, 7H), 4.33- 4.26 (m, 1H), 4.12- 4.02 (m, 3H), 3.26- 3.18 (m, 2H), 2.54- 2.43 (m, 2H), 2.35- 2.27 (m, 1H), 2.15- 2.06 (m, 2H), 2.04- 1.90 (m, 4H), 1.88- 1.78 (m, 3H) |
| 682 | ((2-(((3S,6S,9aS)-3- (3-(5-chloro-4- methoxypyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 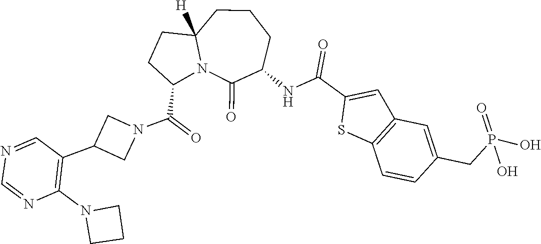 [SMILES: O=C(N[C@H]1CCC[C@H]2CC[C@@H](C(=O)N3CC(c4cncnc4N4CCC4)C3)N2C1=O)c1cc2cc(CP(=O)(O)O)ccc2s1] | 665.2 | ¹H NMR (400 MHz, DMSO-d₆) δ 8.76- 8.67 (m, 1H), 8.54 (t, J = 6.4 Hz, 2H), 8.18 (d, J = 13.6 Hz, 1H), 7.94-7.85 (m, 2H), 7.58-7.47 (m, 1H), 7.34-7.02 (m, 2H), 5.54-5.29 (m, 1H), 4.83-4.58 (m, 2H), 4.48-4.44 (m, 1H), 4.36-4.19 (m, 2H), 4.16-4.08 (m, 1H), 4.04-3.96 (m, 2H), 3.91 (d, J = 3.6 Hz, |
| | | | | 3H), 2.25-2.15 (m, |
| | | | | 1H), 2.06-1.93 (m, |
| | | | | 2H), 1.89-1.76 (m, |
| | | | | 4H), 1.76-1.66 (m, |
| | | | | 3H) |
| 683 | ((2-(((3S,6S,9aS)-3- (3-(4-((1S,4S)-2- oxa-5- azabicyclo[2.2.1] heptan-5-yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 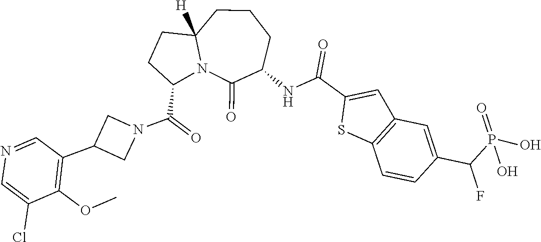 [SMILES: COc1c(Cl)cncc1C1CN(C(=O)[C@@H]2CC[C@@H]3CCC[C@H](NC(=O)c4cc5cc(C(F)P(=O)(O)O)ccc5s4)C(=O)N32)C1] | 698.1 | ¹H NMR (400 MHz, CD₃OD) δ 8.33 (s, 1H), 8.09-7.98 (m, 3H), 7.97 (d, J = 1.6 Hz, 1H), 7.64-7.56 (m, 1H), 7.02-6.95 (m, 1H), 5.91-5.70 (m, 1H), 4.78-4.68 (m, 3H), 4.62-4.33 (m, 4H), 4.29-3.99 (m, 3H), 3.87 ( s, 3H), 3.42 ( d, J = 9.6 Hz, 1H), 2.36-2.26 (m, 1H), 2.17-2.06 (m, 4H), 2.06-1.91 (m, |
| | | | | 4H), 1.90-1.77 (m, |
| | | | | 3H |
| 684 | ((2-(((3S,6S,9aS)-3- (3-(4-(6-oxa-3- azabicyclo[3.1.1] heptan-3-yl)pyridin- 3-yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 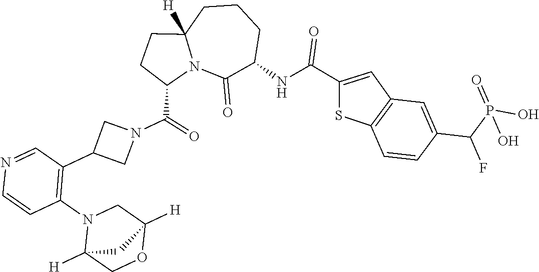 [SMILES: O=C(N[C@H]1CCC[C@H]2CC[C@@H](C(=O)N3CC(c4cnccc4N4C[C@@H]5C[C@H]4CO5)C3)N2C1=O)c1cc2cc(C(F)P(=O)(O)O)ccc2s1] | 698.1 | ¹H NMR (400 MHz, CD₃OD) δ 8.38-8.35 (m, 1H), 8.17-7.97 (m, 3H), 7.95-7.88 (m, 1H), 7.64-7.52 (m, 1H), 7.29-7.14 (m, 1H), 5.89-5.69 (m, 1H), 4.76-4.63 (m, 4H), 4.62-4.44 (m, 3H), 4.42-4.33 (m, 1H), 4.15-4.02 (m, 2H), 3.93-3.76 (m, 4H), 2.36-2.26 (m, 1H), 2.17-2.06 (m, 2H), 2.06-1.95 (m, 4H), 1.92 (d, J = 14.4 Hz, 2H), 1.83 (s, 3H |
| 685 | ((R)-(2- (((3S,6S,9aS)-3-(3- (4-(dimethylamino)- 5-fluoropyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 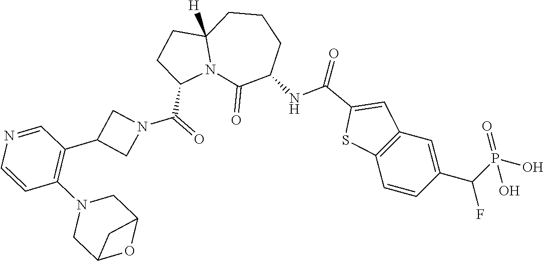 [SMILES: O=C(N[C@H]1CCC[C@H]2CC[C@@H](C(=O)N3CC(c4cnccc4N4CC5CC(C4)O5)C3)N2C1=O)c1cc2cc(C(F)P(=O)(O)O)ccc2s1] | 662.3 | ¹H NMR (400 MHz, CD₃OD) δ 8.32-8.21 (m, 1H), 8.11-8.05 (m, 1H), 8.05-7.99 (m, 1H), 7.91-7.82 (m, 2H), 7.62-7.53 (m, 1H), 5.85-5.64 (m, 1H), 4.76-4.68 (m, 2H), 4.59-4.48 (m, 2H), 4.46-4.36 (m, 1H), 4.32-4.20 (m, 1H), 4.11-4.00 (m, 2H), 3.14-3.01 (m, 6H), 2.37-2.24 (m, 1H), 2.18-2.07 (m, 2H), 2.06-1.98 |
| | | | | (m, 2H), 1.97-1.90 |
| | | | | (m, 2H), 1.87 (d, J = |
| | | | | 10.4 Hz, 2H), 1.83- |
| | | | | 1.78 (m, 1H) |
| 686 | ((2-(((3S,6S,9aS)-3- (3-(4-((S)-3- methoxypyrrolidin- 1-yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 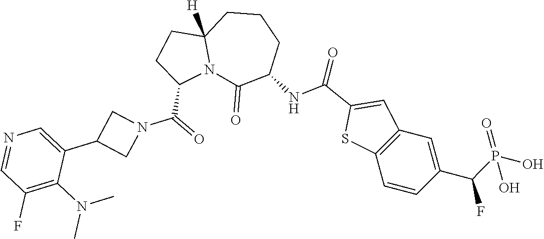 [SMILES: CN(C)c1c(F)cncc1C1CN(C(=O)[C@@H]2CC[C@@H]3CCC[C@H](NC(=O)c4cc5cc([C@H](F)P(=O)(O)O)ccc5s4)C(=O)N32)C1] | 682.4 | ¹H NMR (400 MHz, CD₃OD) δ = 8.32 (d, J = 2.4 Hz, 1H), 8.01- 7.89 (m, 2H), 7.86- 7.79 (m, 2H), 7.44 (t, J = 8.4 Hz, 1H), 6.91- 6.77 (m, 1H), 4.79- 4.72 (m, 1H), 4.69- 4.57 (m, 1H), 4.56- 4.33 (m, 4H), 4.17- 4.05 (m, 3H), 3.88- 3.75 (m, 2H), 3.64- 3.58 (m, 1H), 3.56- 3.49 (m, 1H), 3.36 (d, J = 11.2 Hz, 3H), 3.28- |
| | | | | 3.20 (m, 2H), 2.36- |
| | | | | 2.22 (m, 2H), 2.17- |
| | | | | 2.09 (m, 2H), 2.07- |
| | | | | 1.96 (m, 4H), 1.94- |
| | | | | 1.79 (m, 4H) |
| 687 | ((2-(((3S,6S,9aS)-3- (3-(4-((R)-3- fluoropyrrolidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 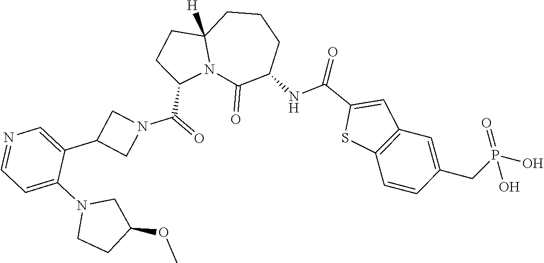 [SMILES: CO[C@H]1CCN(c2ccncc2C2CN(C(=O)[C@@H]3CC[C@@H]4CCC[C@H](NC(=O)c5cc6cc(CP(=O)(O)O)ccc6s5)C(=O)N43)C2)C1] | 670.2 | ¹H NMR (400 MHz, CD₃OD) δ = 8.38 (s, 1H), 8.15-7.93 (m, 2H), 7.90-7.78 (m, 2H), 7.49-7.40 (m, 1H), 7.03-6.90 (m, 1H), 5.56-5.33 (m, 1H), 4.81-4.69 (m, 2H), 4.67-4.53 (m, 2H), 4.52-4.45 (m, 1H), 4.38-4.28 (m, 1H), 4.19-4.06 (m, 2H), 4.05-3.87 (m, 2H), 3.87-3.73 (m, 2H), 3.29-3.21 (m, 2H), 2.50-2.38 (m, |
| | | | | 1H), 2.36-2.29 (m, |
| | | | | 1H), 2.24-2.06 (m, |
| | | | | 3H), 2.06-1.90 (m, |
| | | | | 4H), 1.90-1.76 (m, |
| | | | | 3H |
| 688 | ((2-(((3S,6S,9aS)-3- (3-(4-((R)-3- methoxypyrrolidin- 1-yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 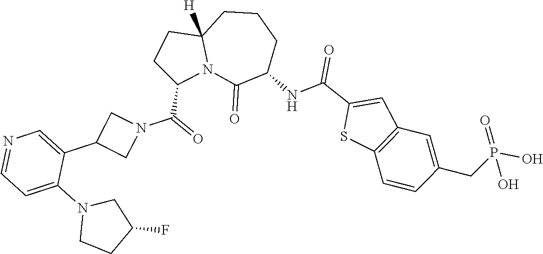 [SMILES: O=C(N[C@H]1CCC[C@H]2CC[C@@H](C(=O)N3CC(c4cnccc4N4CC[C@@H](F)C4)C3)N2C1=O)c1cc2cc(CP(=O)(O)O)ccc2s1] | 682.2 | ¹H NMR (400 MHz, CD₃OD) δ 7.91-7.89 (m, 1H), 7.84-7.78 (m, 1H), 7.67-7.62 (m, 2H), 7.43-7.38 (m, 2H), 6.44-6.43 (m, 1H), 4.74-4.28 (m, 2H), 4.07-4.06 (m, 2H), 4.05-4.04 (m, 1H), 3.50-3.36 (m, 4H), 3.33-3.32 (m, 1H), 3.31-3.30 (m, 3H), 3.29 3.27(m, 3H), 3.26- 3.15 (m, 2H), 2.31- 2.10 (m, 1H), 2.04- |
| | | | | 2.01 (m, 4H), 1.97- |
| | | | | 1.91 (m, 4H), 1.89- |
| | | | | 1.88 (m, 3H) |
| 689 | ((R)-(2- (((3S,6S,9aS)-3-(3- (4-(azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 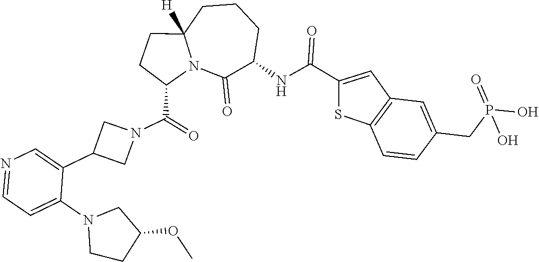 [SMILES: CO[C@@H]1CCN(c2ccncc2C2CN(C(=O)[C@@H]3CC[C@@H]4CCC[C@H](NC(=O)c5cc6cc(CP(=O)(O)O)ccc6s5)C(=O)N43)C2)C1] | 657.2 | ¹H NMR (400 MHz, CD₃OD) δ 8.43-8.35 (m, 1H), 8.22-8.11 (m, 1H), 8.03 (d, J = 7.6 Hz, 1H), 8.00 (s, 1H), 7.91 (dd, J = 3.6, 8.4 Hz, 1H), 7.58 (d, J = 8.4 Hz, 1H), 5.87- 5.71 (m, 1H), 4.82- 4.70 (m, 2H), 4.62- 4.47 (m, 5H), 4.45- 4.37 (m, 1H), 4.32- 4.23 (m, 1H), 4.12- 3.99 (m, 3H), 2.54- 2.42 (m, 2H), 2.35- 2.26 (m, 1H), 2.15- |
| | | | | 2.00 (m, 3H), 1.98- |
| | | | | 1.78 (m, 6H) |
| 690 | ((S)-(2- (((3S,6S,9aS)-3-(3- (4-(azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 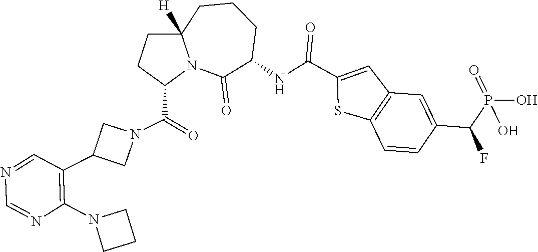 [SMILES: O=C(N[C@H]1CCC[C@H]2CC[C@@H](C(=O)N3CC(c4cncnc4N4CCC4)C3)N2C1=O)c1cc2cc([C@H](F)P(=O)(O)O)ccc2s1] | 657.2 | ¹H NMR (400 MHz, CD₃OD) δ 8.45-8.35 (m, 1H), 8.23-8.11 (m, 1H), 8.03 (d, J = 4.8 Hz, 1H), 7.99 (d, J = 6.8 Hz, 1H), 7.92 (d, J = 8.4 Hz, 1H), 7.60 (t, J = 8.8 Hz, 1H), 5.86-5.71 (m, 1H), 4.83-4.70 (m, 2H), 4.62-4.46 (m, 5H), 4.40 (t, J = 9.2 Hz, 1H), 4.32-4.23 (m, 1H), 4.11-4.00 (m, 3H), 2.54-2.41 |
| | | | | (m, 2H), 2.35-2.26 |
| | | | | (m, 1H), 2.14-2.00 |
| | | | | (m, 3H), 1.99-1.78 |
| | | | | (m, 6H) |
| 691 | ((2-(((3S,6S,9aS)-3- (3-(4-(3- (difluoromethyl) azetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 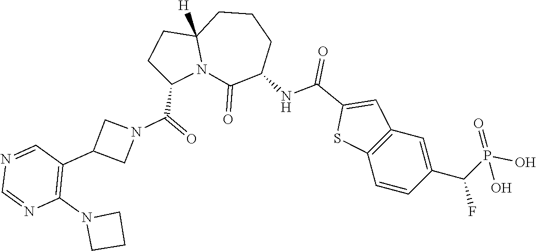 [SMILES: O=C(N[C@H]1CCC[C@H]2CC[C@@H](C(=O)N3CC(c4cncnc4N4CCC4)C3)N2C1=O)c1cc2cc([C@@H](F)P(=O)(O)O)ccc2s1] | 688.4 | ¹H NMR (400 MHz, CD₃OD) δ 8.36-8.24 (m, 1H), 8.03-7.93 (m, 2H), 7.83 (d, J = 8.4 Hz, 2H), 7.42 (d, J = 8.4 Hz, 1H), 6.57 (t, J = 7.2 Hz, 1H), 6.39-6.03 (m, 1H), 4.76-4.72 (m, 1H), 4.66-4.56 (m, 2H), 4.55-4.52 (m, 3H), 4.48-4.25 (m, 4H), 4.23-4.14 (m, 1H), 4.12-4.04 (m, 2H), 3.26 (s, 1H), 3.21 (s, |
| | | | | 1H), 2.38-2.25 (m, |
| | | | | 1H), 2.21-2.06 (m, |
| | | | | 2H), 2.06-2.00 (m, |
| | | | | 1H), 2.00-1.96 (m, |
| | | | | 1H), 1.94 (d, J = 5.4 |
| | | | | Hz, 1H), 1.90 (d, J = |
| | | | | 10.4 Hz, 1H), 1.87- |
| | | | | 1.76 (m, 3H) |
| 692 | ((2-(((3S,6S,9aS)-5- oxo-3-(3-(4-(3- (trifluoromethyl) azetidin-1- yl)pyridin-3- yl)azetidine-1- carbonyl)octahydro- 1H-pyrrolo[1,2- a]azepin-6- yl)carbamoyl)benzo [b]thiophen-5- yl)methyl) phosphonic acid | 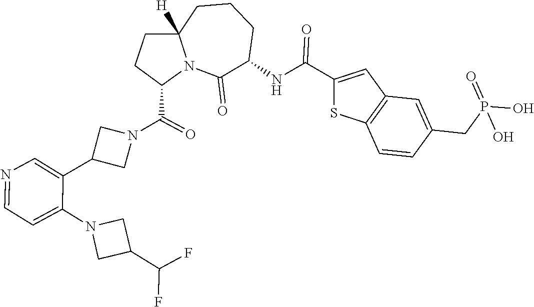 [SMILES: O=C(N[C@H]1CCC[C@H]2CC[C@@H](C(=O)N3CC(c4cnccc4N4CC(C(F)F)C4)C3)N2C1=O)c1cc2cc(CP(=O)(O)O)ccc2s1] | 706.2 | ¹H NMR (400 MHz, CD₃OD) δ 8.35 (d, J = 15.6 Hz, 1H), 8.05 (dd, J = 7.6, 10.4 Hz, 1H), 8.02-7.97 (m, 1H), 7.88-7.81 (m, 2H), 7.49-7.38 (m, 1H), 6.67-6.59 (m, 1H), 4.77-4.70 (m, 1H), 4.69-4.59 (m, 3H), 4.59-4.49 (m, 3H), 4.42 (d, J = 9.6 Hz, 1H), 4.33 (q, J = 8.8 Hz, 1H), 4.20- 4.13 (m, 1H), 4.10 (td, J = 3.6, 9.6 Hz, 2H), 3.78-3.66 (m, 1H), 3.26 (s, 1H), 3.21 (s, 1H), 2.39- |
| | | | | 2.25 (m, 1H), 2.20- |
| | | | | 2.12 (m, 1H), 2.11- |
| | | | | 1.99 (m, 3H), 1.99- |
| | | | | 1.91 (m, 2H), 1.89 (s, |
| | | | | 1H), 1.85 (d, J = 6.8 |
| | | | | Hz, 2H) |
| 693 | ((2-(((3S,6S,9aS)-3- (3-(3- (dimethylamino) pyridin-2-yl)azetidine- 1-carbonyl)-5- oxooctahydro-1H- pyrrolo[1,2- a]azepin-6- yl)carbamoyl)benzo [b]thiophen-5- yl)fluoromethyl) phosphonic acid | 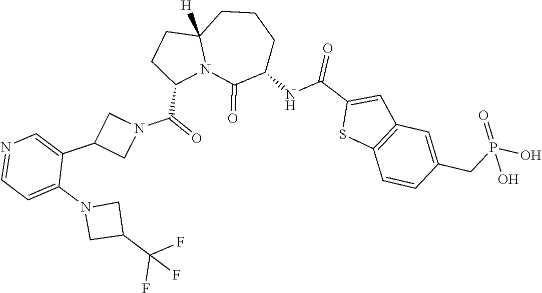 [SMILES: O=C(N[C@H]1CCC[C@H]2CC[C@@H](C(=O)N3CC(c4cnccc4N4CC(C(F)(F)F)C4)C3)N2C1=O)c1cc2cc(CP(=O)(O)O)ccc2s1] | 644.6 | ¹H NMR (400 MHz, DMSO-d₆) δ 8.68 (s, 1H), 8.56-8.45 (m, 1H), 8.29-8.20 (m, 1H), 8.10 (d, J = 12.4 Hz, 1H), 7.95-7.85 (m, 2H), 7.58-7.49 (m, 1H), 7.42 (d, J = 8.0 Hz, 1H), 5.89- 5.46 (m, 1H), 4.56- 4.47 (m, 1H), 4.32 (s, 1H), 3.89 (d, J = 3.6 Hz, 1H), 3.76-3.70 (m, 1H), 3.56 (s, 2H), 3.40 (s, 1H), 3.37 (s, 1H), 2.41 (s, 6H), 2.13-2.03 (m, 1H), 1.95-1.75 (m, 4H), |
| | | | | 1.71-1.67 (m, 1H), |
| | | | | 1.67-1.56 (m, 4H) |
| 694 | ((2-(((3S,6S,9aS)-3- (3-(4-((1R,4R)-2- oxa-5- azabicyclo[2.2.1] heptan-5-yl)pyridin- 3-yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 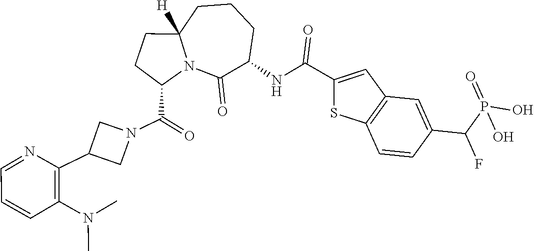 [SMILES: CN(C)c1cccnc1C1CN(C(=O)[C@@H]2CC[C@@H]3CCC[C@H](NC(=O)c4cc5cc(C(F)P(=O)(O)O)ccc5s4)C(=O)N32)C1] | 698.2 | ¹H NMR (400 MHz, DMSO-d₆) 8.71 8.64 (m, 1H), 8.42 (s, 1H), 8.23-8.08 (m, 2H), 8.01-7.95 (m, 2H), 7.59-7.46 (m, 1H), 7.14-6.92 (m, 1H), 5.89-5.60 (m, 1H), 4.82 (s, 1H), 4.69-4.58 (m, 3H), 4.52-4.39 (m, 2H), 4.32-4.26 (m, 1H), 4.21 (d, J = 7.6 Hz, 1H), 4.14 (d, J = 7.2 Hz, 1H), 3.99 (d, J = |
| | | | | 4.8 Hz, 2H), 3.92 (s, |
| | | | | 1H), 3.80 (d, J = 8.8 |
| | | | | Hz, 3H), 3.33 (d, J = |
| | | | | 9.6 Hz, 1H), 2.32- |
| | | | | 2.14 (m, 1H), 2.10- |
| | | | | 1.92 (m, 4H), 1.92- |
| | | | | 1.52 (m, 7H) |
| 695 | ((2-(((3S,6S,9aS)-3- (3-(4- (cyclopentyloxy) pyridin-3- yl)azetidine- 1-carbonyl)-5- oxooctahydro-1H- pyrrolo[1,2- a]azepin-6- yl)carbamoyl)benzo [b]thiophen-5- yl)fluoromethyl) phosphonic acid | 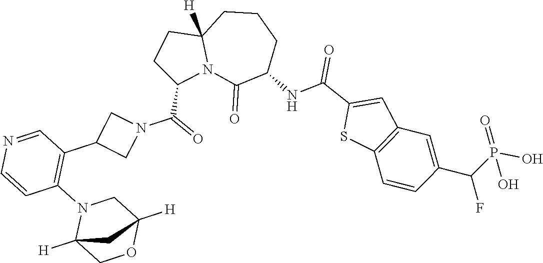 [SMILES: O=C(N[C@H]1CCC[C@H]2CC[C@@H](C(=O)N3CC(c4cnccc4N4C[C@H]5C[C@@H]4CO5)C3)N2C1=O)c1cc2cc(C(F)P(=O)(O)O)ccc2s1] | 685.2 | ¹H NMR (400 MHz, DMSO-d₆) δ 8.79- 8.62 (m, 1H), 8.60- 8.42 (m, 2H), 8.27- 8.12 (m, 1H), 8.08- 7.92 (m, 2H), 7.58- 7.48 (m, 1H), 7.41- 7.28 (m, 1H), 5.92- 5.71 (m, 1H), 5.13 (d, J = 16.8 Hz, 1H), 4.72- 4.53 (m, 2H), 4.46- 4.30 (m, 2H), 4.21- 4.13 (m, 1H), 4.08- 3.96 (m, 3H), 2.27- 2.10 (m, 1H), 2.05- 1.89 (m, 4H), 1.88- 1.54 (m, 13H) |
| 696 | ((S)-(2- (((3S,6S,9aS)-3-(3-(4- (dimethylcarbamoyl) pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 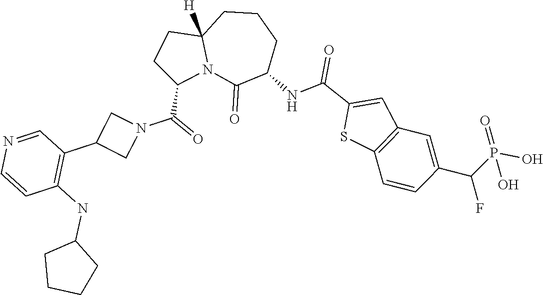 [SMILES: O=C(N[C@H]1CCC[C@H]2CC[C@@H](C(=O)N3CC(c4cnccc4NC4CCCC4)C3)N2C1=O)c1cc2cc(C(F)P(=O)(O)O)ccc2s1] | 672.2 | ¹H NMR (400 MHz, CD₃OD) δ 9.11-8.84 (m, 1H), 8.74-8.53 (m, 1H), 8.07 (d, J = 7.6 Hz, 1H), 8.02 (s, 1H), 7.96-7.90 (m, 1H), 7.67-7.52 (m, 2H), 6.02-5.52 (m, 1H), 4.77-4.71 (m, 1H), 4.70-4.61 (m, 1H), 4.57-4.52 (m, 1H), 4.52-4.25 (m, 2H), 4.19-4.12 (m, 1H), 4.08-4.06 (m, 2H), 3.17-3.08 (m, 3H), 2.86 (s, 3H), 2.37-2.25 (m, 1H), 2.19-2.06 (m, 2H), |
| | | | | 2.05 (s, 2H), 1.98- |
| | | | | 1.90 (m, 2H), 1.90- |
| | | | | 1.77 (m, 3H) |
| 697 | ((2-(((3S,6S,9aS)-3- (3-(3,6-dihydro-2H- pyran-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 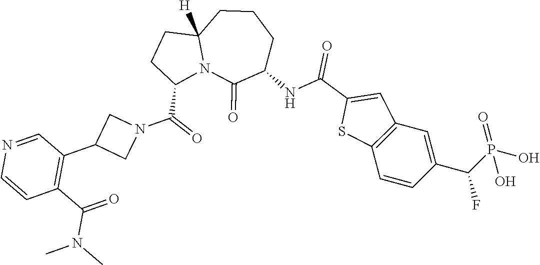 [SMILES: CN(C)C(=O)c1ccncc1C1CN(C(=O)[C@@H]2CC[C@@H]3CCC[C@H](NC(=O)c4cc5cc([C@@H](F)P(=O)(O)O)ccc5s4)C(=O)N32)C1] | 588.3 | ¹H NMR (400 MHz, CD₃OD) δ 8.01 (d, J = 2.4 Hz, 1H), 7.85 (d, J = 5.2 Hz, 2H), 7.43 (d, J = 8.4 Hz, 1H), 5.66 (s, 1H), 4.77-4.70 (m, 1H), 4.62-4.52 (m, 1H), 4.46-4.28 (m, 1H), 4.22-4.12 (m, 3H), 4.11-4.01 (m, 2H), 3.97-3.85 (m, 1H), 3.85-3.75 (m, 2H), 3.34 (d, J = 8.8 Hz, 1H), 3.28 (s, 1H), 3.23 (s, 1H), 2.33- |
| | | | | 2.25 (m, 1H), 2.19- |
| | | | | 1.98 (m, 5H), 1.96- |
| | | | | 1.75 (m, 6H) |
| 698 | ((S)-(2- (((3S,6S,9aS)-3-(3- (2-chloro-3- methoxypyridin-4- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 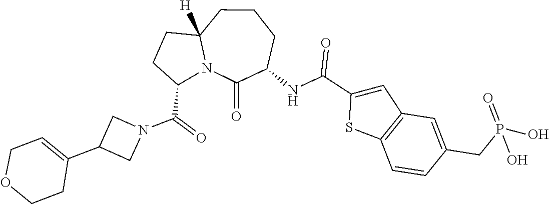 [SMILES: O=C(N[C@H]1CCC[C@H]2CC[C@@H](C(=O)N3CC(C4=CCOCC4)C3)N2C1=O)c1cc2cc(CP(=O)(O)O)ccc2s1] | 665.1 | ¹H NMR (400 MHz, CD₃OD) δ 8.16-8.10 (m, 1H), 8.11-8.06 (m, 1H), 8.03 (s, 1H), 7.98-7.92 (m, 1H), 7.63-7.59 (m, 1H), 7.58 (s, 1H), 5.95- 5.70 (m, 1H), 4.78- 4.66 (m, 2H), 4.62- 4.46 (m, 2H), 4.44- 4.24 (m, 2H), 4.11- 4.01 (m, 2H), 3.86 (s, 3H), 2.40-2.22 (m, |
| | | | | 1H), 2.20-2.05 (m, |
| | | | | 2H), 2.03-1.96 (m, |
| | | | | 2H), 1.96-1.90 (m, |
| | | | | 2H), 1.89 (s, 3H) |
| 699 | (fluoro(2- (((3S,6S,9aS)-5- oxo-3-(3-(4- phenoxypyridin-3- yl)azetidine-1- carbonyl)octahydro- 1H-pyrrolo[1,2- a]azepin-6- yl)carbamoyl)benzo [b]thiophen-5- yl)methyl) phosphonic acid | 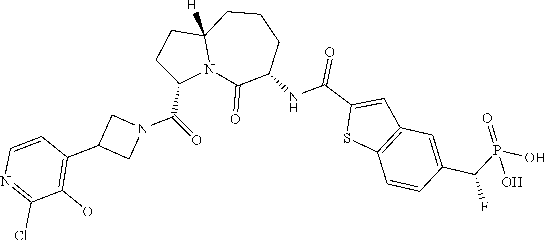 [SMILES: O=C(N[C@H]1CCC[C@H]2CC[C@@H](C(=O)N3CC(c4ccnc(Cl)c4O)C3)N2C1=O)c1cc2cc([C@@H](F)P(=O)(O)O)ccc2s1] | 693.2 | ¹H NMR (400 MHz, DMSO-d₆) δ 8.76- 8.66 (m, 2H), 8.51- 8.41 (m, 1H), 8.23 (d, J = 13.6 Hz, 1H), 8.03 (d, J = 8.4 Hz, 1H), 7.97 (s, 1H), 7.55- 7.48 (m, 3H), 7.38- 7.30 (m, 1H), 7.24 (t, J = 8.8 Hz, 2H), 6.80 (dd, J = 6.0, 16.8 Hz, 1H), 5.90-5.75 (m, 1H), 4.78 (t, J = 8.0 Hz, 1H), 4.67-4.59 (m, 2H), 4.54-4.47 |
| | | | | (m, 1H), 4.45 (dd, J = |
| | | | | 4.0, 7.6 Hz, 1H), 4.30- |
| | | | | 4.25 (m, 1H), 4.23- |
| | | | | 4.17 (m, 2H), 4.01- |
| | | | | 3.95 (m, 2H), 2.23- |
| | | | | 2.11 (m, 1H), 2.08- |
| | | | | 2.00 (m, 1H), 1.96- |
| | | | | 1.93 (m, 1H), 1.88- |
| | | | | 1.80 (m, 2H), 1.78- |
| | | | | 1.63 (m, 5H) |
| 700 | ((S)-fluoro(2- (((3S,6S,9aS)-3-(3- (3-fluoro-2- methoxypyridin-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 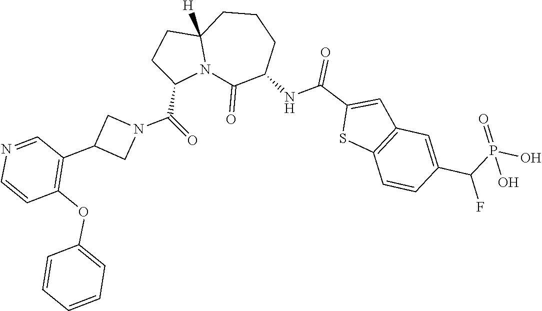 [SMILES: O=C(N[C@H]1CCC[C@H]2CC[C@@H](C(=O)N3CC(c4cnccc4Oc4ccccc4)C3)N2C1=O)c1cc2cc(C(F)P(=O)(O)O)ccc2s1] | 649.2 | ¹H NMR (400 MHz, CD₃OD) δ 8.09 (d, J = 8.0 Hz, 1H), 8.06- 8.02 (m, 1H), 7.99- 7.94 (m, 1H), 7.93- 7.89 (m, 1H), 7.63- 7.52 (m, 1H), 7.25- 6.99 (m, 1H), 5.96- 5.71 (m, 1H), 4.98- 4.88 (m, 1H), 4.75 (d, J = 8.0 Hz, 1H), 4.72- 4.64 (m, 1H), 4.60- 4.54 (m, 1H), 4.43- 4.32 (m, 1H), 4.28- 4.14 (m, 1H), 4.11- 4.05 (m, 2H), 3.98 (d, J = 8.8 Hz, 3H), 2.37- 2.26 (m, 1H), 2.20- 2.06 (m, 2H), 2.05- |
| | | | | 1.94 (m, 3H), 1.93- |
| | | | | 1.86 (m, 2H), 1.85- |
| | | | | 1.74 (m, 2H) |
| 701 | ((S)-(2- (((3S,6S,9aS)-3-(3- (2-(dimethylamino) pyridin-4-yl)azetidine- 1-carbonyl)-5- oxooctahydro-1H- pyrrolo[1,2- a]azepin-6- yl)carbamoyl)benzo [b]thiophen-5- yl)fluoromethyl) phosphonic acid | 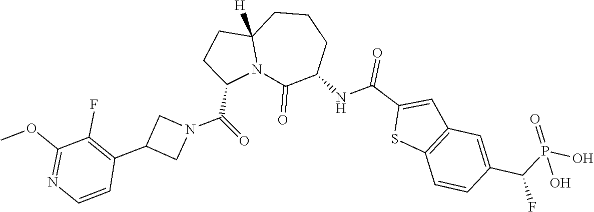 [SMILES: COc1nccc(C2CN(C(=O)[C@@H]3CC[C@@H]4CCC[C@H](NC(=O)c5cc6cc([C@@H](F)P(=O)(O)O)ccc6s5)C(=O)N43)C2)c1F] | 644.3 | ¹H NMR (400 MHz, DMSO-d₆) δ 8.81- 8.66 (m, 1H), 8.30- 8.22 (m, 1H), 8.04 (dd, J = 5.6, 8.4 Hz, 1H), 8.00-7.84 (m, 2H), 7.58-7.47 (m, 1H), 7.10-6.88 (m, 1H), 5.95-5.72 (m, 1H), 4.73-4.54 (m, 2H), 4.49-4.35 (m, 2H), 4.29-4.21 (m, 1H), 4.04-3.92 (m, 3H), 3.90-3.83 (m, |
| | | | | 2H), 3.22-3.04 (m, |
| | | | | 6H), 2.29-2.14 (m, |
| | | | | 1H), 2.10-1.94 (m, |
| | | | | 2H), 1.92-1.78 (m, |
| | | | | 4H), 1.75-1.62 (m, |
| | | | | 3H) |
| 702 | ((S)-fluoro(2- (((3S,6S,9aS)-3-(3- (2-methoxypyridin- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 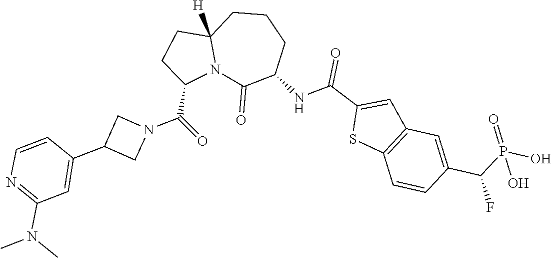 [SMILES: CN(C)c1cc(C2CN(C(=O)[C@@H]3CC[C@@H]4CCC[C@H](NC(=O)c5cc6cc([C@@H](F)P(=O)(O)O)ccc6s5)C(=O)N43)C2)ccn1] | 631.1 | ¹H NMR (400 MHz, DMSO-d₆) δ 8.83- 8.76 (m, 1H), 8.26 (d, J = 4.8 Hz, 1H), 8.16- 8.10 (m, 1H), 8.05 (d, J = 8.4 Hz, 1H), 7.99 (s, 1H), 7.54 (d, J = 8.4 Hz, 1H), 7.01 (t, J = 5.6 Hz, 1H), 6.82 (d, J = 2.0 Hz, 1H), 5.92-5.78 (m, 1H), 4.81-4.63 (m, 2H), 4.57 (t, J = 8.4 Hz, 1H), 4.47-4.33 (m, 2H), 4.28-4.19 (m, 1H), 4.05-3.96 |
| | | | | (m, 1H), 3.92-3.71 |
| | | | | (m, 5H), 2.26-2.13 |
| | | | | (m, 1H), 2.08-1.92 |
| | | | | (m, 2H), 1.90-1.64 |
| | | | | (m, 7H) |
| 703 | ((R)-fluoro(2- (((3S,6S,9aS)-3-(3- (5-fluoro-4- (methylamino) pyridin-3-yl) azetidine-1- carbonyl)-5- oxooctahydro-1H- pyrrolo[1,2- a]azepin-6- yl)carbamoyl)benzo [b]thiophen-5- yl)methyl) phosphonic acid | 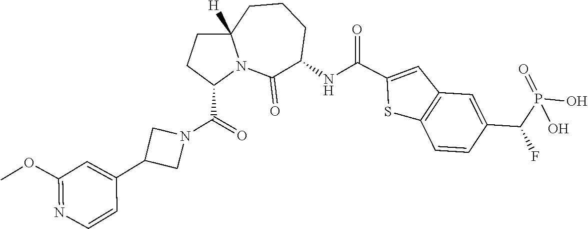 [SMILES: COc1cc(C2CN(C(=O)[C@@H]3CC[C@@H]4CCC[C@H](NC(=O)c5cc6cc([C@@H](F)P(=O)(O)O)ccc6s5)C(=O)N43)C2)ccn1] | 648.2 | ¹H NMR (400 MHz, CD₃OD) δ 8.28-8.20 (m, 1H), 8.15-8.08 (m, 1H), 8.06-7.96 (m, 2H), 7.95-7.89 (m, 1H), 7.63-7.54 (m, 1H), 5.88-5.67 (m, 1H), 4.79-4.66 (m, 2H), 4.61-4.48 (m, 2H), 4.41 (t, J = 9.2 Hz, 1H), 4.14- 4.00 (m, 2H), 3.97- 3.83 (m, 1H), 3.26 (dd, J = 2.4, 7.2 Hz, |
| | | | | 3H), 2.37-2.26 (m, |
| | | | | 1H), 2.19-2.06 (m, |
| | | | | 2H), 2.05-1.90 (m, |
| | | | | 4H), 1.89-1.76 (m, |
| | | | | 3H) |
| 704 | ((2-(((3S,6S,9aS)-3- (3-(4-(2-oxa-6- azaspiro[3.3]heptan- 6-yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 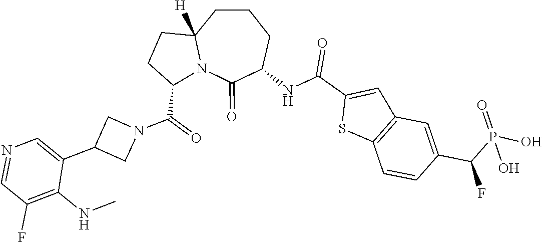 [SMILES: CNc1c(F)cncc1C1CN(C(=O)[C@@H]2CC[C@@H]3CCC[C@H](NC(=O)c4cc5cc([C@H](F)P(=O)(O)O)ccc5s4)C(=O)N32)C1] | 680.1 | ¹H NMR (400 MHz, DMSO-d₆) δ 8.77- 8.58 (m, 1H), 8.12- 8.00 (m, 2H), 7.82- 7.66 (m, 2H), 7.32- 7.21 (m, 1H), 6.36 (d, J = 6.0 Hz, 1H), 4.72- 4.65 (m, 4H), 4.64- 4.60 (m, 1H), 4.59- 4.52 (m, 1H), 4.49- 4.42 (m, 1H), 4.41- 4.34 (m, 1H), 4.25 (d, J = 3.6 Hz, 1H), 4.19 (s, 2H), 4.15 (d, J = 5.4 Hz, 1H), 4.03- |
| | | | | 3.95 (m, 2H), 3.94- |
| | | | | 3.85 (m, 2H), 2.95- |
| | | | | 2.87 (m, 2H), 2.22- |
| | | | | 2.16 (m, 1H), 2.04- |
| | | | | 1.93 (m, 2H), 1.89- |
| | | | | 1.78 (m, 4H), 1.70 (d, |
| | | | | J = 10.4 Hz, 3H) |
| 705 | (fluoro(2- (((3S,6S,9aS)-5- oxo-3-(3-(4- (pyridin-4- ylmethoxy)pyridin- 3-yl)azetidine-1- carbonyl)octahydro- 1H-pyrrolo[1,2- a]azepin-6- yl)carbamoyl)benzo [b]thiophen-5- yl)methyl) phosphonic acid | 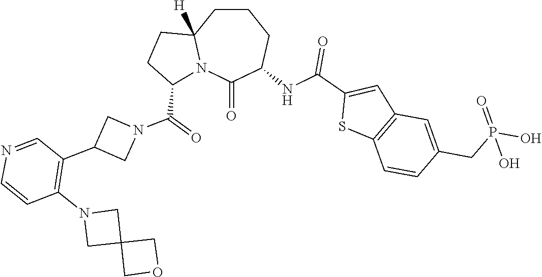 [SMILES: O=C(N[C@H]1CCC[C@H]2CC[C@@H](C(=O)N3CC(c4cnccc4N4CC5(COC5)C4)C3)N2C1=O)c1cc2cc(CP(=O)(O)O)ccc2s1] | 708.2 | ¹H NMR (400 MHz, DMSO-d₆) δ 8.71 ( dd, J = 7.6, 12.4 Hz, 1H), 8.57 (dd, J = 5.2, 9.6 Hz, 2H), 8.35 ( d, J = 6.0 Hz, 2H), 8.42 (d, J = 9.6 Hz, 1H), 8.03-7.92 (m, 2H), 7.55-7.39 (m, 3H), 7.20-7.02 (m, 1H), 5.88-5.66 (m, 1H), 5.31 (s, 2H), 4.72 ( t, J = 8.4 Hz, 1H), 4.67- 4.60 (m, 1H), 4.59- 4.50 (m, 1H), 4.46- 4.34 (m, 2H), 4.28- 4.21 (m, 1H), 4.10 ( s, |
| | | | | 1H), 3.98-3.95 (m, |
| | | | | 1H), 2.24-2.09 (m, |
| | | | | 1H), 2.04-2.01 (m, |
| | | | | 1H), 1.90-1.79 (m, |
| | | | | 3H), 1.78-1.66 (m, |
| | | | | 4H), 1.65-1.56 (m, |
| | | | | 1H) |
| 706 | (fluoro(2- (((3S,6S,9aS)-3- ((S)-3-(4- methoxypyridin-3- yl)pyrrolidine-1- carbonyl)-5- oxooctahydro-1H- pyrrolo[1,2- a]azepin-6- yl)carbamoyl)benzo [b]thiophen-5- yl)methyl) phosphonic acid | 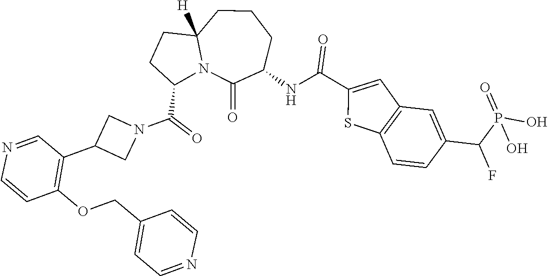 [SMILES: O=C(N[C@H]1CCC[C@H]2CC[C@@H](C(=O)N3CC(c4cnccc4OCc4ccncc4)C3)N2C1=O)c1cc2cc(C(F)P(=O)(O)O)ccc2s1] | 645.3 | ¹H NMR (400 MHz, DMSO-d₆) δ 8.70 (t, J = 7.6 Hz, 1H), 8.61 (dd, J = 6.0, 10.0 Hz, 1H), 8.45 (d, J = 11.2 Hz, 1H), 8.27-8.19 (m, 1H), 8.02 (dd, J = 5.2, 8.4 Hz, 1H), 7.96 (s, 1H), 7.55-7.48 (m, 1H), 7.40 (dd, J = 6.4, 10.0 Hz, 1H), 5.91-5.71 (m, 1H), 4.66 (d, J = 8.8 Hz, 2H), 4.03 (d, J = 5.2 Hz, 3H), 4.01-3.97 (m, 1H), 3.91-3.87 (m, 1H), 3.71 (s, 1H), |
| | | | | 3.68 (s, 1H), 3.55 (dd, |
| | | | | J = 4.0, 7.6 Hz, 1H), |
| | | | | 3.46 (d, J = 14.4 Hz, |
| | | | | 1H), 3.33 (dd, J = 4.0, |
| | | | | 8.0 Hz, 1H), 3.22 (dd, |
| | | | | J = 9.6, 10.8 Hz, 1H), |
| | | | | 2.36-2.25 (m, 1H), |
| | | | | 2.25-2.15 (m, 2H), |
| | | | | 2.13-2.04 (m, 1H), |
| | | | | 2.02-1.88 (m, 2H), |
| | | | | 1.86-1.76 (m, 4H), |
| | | | | 1.72 (d, J = 13.6 Hz, |
| | | | | 2H) |
| 707 | (fluoro(2- (((3S,6S,9aS)-3- ((R)-3-(4- methoxypyridin-3- yl)pyrrolidine-1- carbonyl)-5- oxooctahydro-1H- pyrrolo[1,2- a]azepin-6- yl)carbamoyl)benzo [b]thiophen-5- yl)methyl) phosphonic acid | 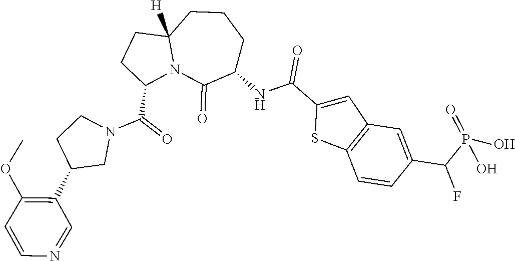 [SMILES: COc1ccncc1[C@@H]1CCN(C(=O)[C@@H]2CC[C@@H]3CCC[C@H](NC(=O)c4cc5cc(C(F)P(=O)(O)O)ccc5s4)C(=O)N32)C1] | 645.2 | ¹H NMR (400 MHz, DMSO-d₆) δ 8.76- 8.66 (m, 1H), 8.65- 8.61 (m, 1H), 8.51- 8.40 (m, 1H), 8.22 (d, J = 8.8 Hz, 1H), 8.03 (d, J = 8.4 Hz, 1H), 7.97 (s, 1H), 7.52 (d, J = 8.4 Hz, 1H), 7.47- 7.40 (m, 1H), 5.91- 5.74 (m, 1H), 4.73- 4.59 (m, 2H), 4.17- 4.08 (m, 1H), 4.04 (d, J = 12.0 Hz, 3H), 4.00 (d, J = 7.2 Hz, 1H), 3.89-3.81 (m, 1H), |
| | | | | 3.81-3.74 (m, 1H), |
| | | | | 3.59 (s, 1H), 3.47 (d, |
| | | | | J = 9.6 Hz, 1H), 3.34 |
| | | | | (d, J = 9.6 Hz, 1H), |
| | | | | 2.33-2.12 (m, 3H), |
| | | | | 2.08-1.94 (m, 2H), |
| | | | | 1.91-1.67 (m, 7H) |
| 708 | ((S)-fluoro(2- (((3S,6S,9aS)-3-(3- (1-methyl-2-oxo- 1,2-dihydropyridin- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 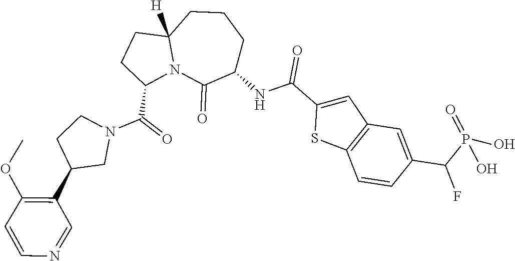 [SMILES: COc1ccncc1[C@H]1CCN(C(=O)[C@@H]2CC[C@@H]3CCC[C@H](NC(=O)c4cc5cc(C(F)P(=O)(O)O)ccc5s4)C(=O)N32)C1] | 631.2 | ¹H NMR (400 MHz, CD₃OD) δ 8.13-8.02 (m, 2H), 7.88 (d, J = 8.00 Hz, 1H), 7.69- 7.51 (m, 2H), 6.67- 6.41 (m, 2H), 5.76- 5.57 (m, 1H), 4.74 (m, 1H), 4.64-4.49 (m, 2H), 4.48-4.15 (m, 2H), 4.11-3.90 (m, 2H), 3.86-3.74 (m, 1H), 3.58-3.46 (m, 3H), 2.35-2.24 (m, 1H), 2.17-2.06 (m, 2H), 1.98 (s, 2H), 1.96-1.90 (m, 2H), |
| | | | | 1.90-1.77 (m, 3H) |
| 709 | ((S)-(2- (((3S,6S,9aS)-3-(3- (2-ethoxypyridin-4- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 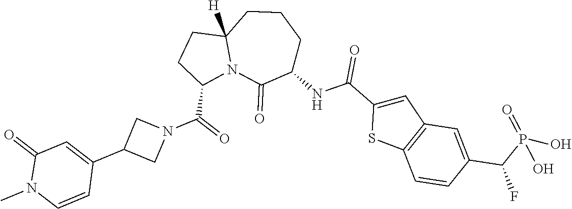 [SMILES: Cn1ccc(C2CN(C(=O)[C@@H]3CC[C@@H]4CCC[C@H](NC(=O)c5cc6cc([C@@H](F)P(=O)(O)O)ccc6s5)C(=O)N43)C2)cc1=O] | 645.2 | ¹H NMR (400 MHz, CD₃OD) δ 8.16-8.01 (m, 3H), 7.96 (d, J = 2.8, 8.6 Hz, 1H), 7.65- 7.55 (m, 1H), 7.38- 7.27 (m, 1H), 7.18- 7.08 (m, 1H), 5.92- 5.74 (m, 1H), 4.78- 4.73 (m, 1H), 4.72- 4.55 (m, 2H), 4.55- 4.44 (m, 1H), 4.43- 4.28 (m, 3H), 4.13- 3.94 (m, 3H), 2.37- |
| | | | | 2.26 (m, 1H), 2.19- |
| | | | | 2.06 (m, 2H), 2.05- |
| | | | | 1.94 (m, 3H), 1.94- |
| | | | | 1.76 (m, 4H), 1.46- |
| | | | | 1.29 (m, 3H) |
| 710 | ((S)-(2- (((3S,6S,9aS)-3-(3- (2-(dimethylamino) pyrimidin-4- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 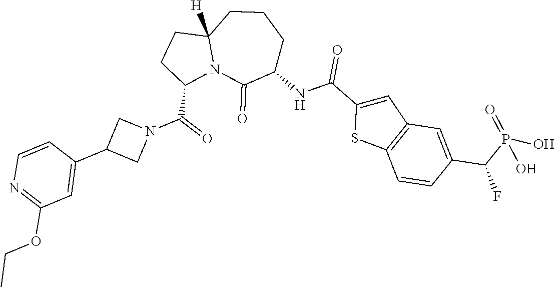 [SMILES: CCOc1cc(C2CN(C(=O)[C@@H]3CC[C@@H]4CCC[C@H](NC(=O)c5cc6cc([C@@H](F)P(=O)(O)O)ccc6s5)C(=O)N43)C2)ccn1] | 645.5 | ¹H NMR (400 MHz, CD₃OD) δ 8.13-7.98 (m, 3H), 7.94-7.89 (m, 1H), 7.65-7.56 (m, 1H), 6.88-6.59 (m, 1H), 5.89-5.70 (m, 1H), 4.82-4.76 (m, 1H), 4.72 (d, J = 12.0 Hz, 1H), 4.60- 4.53 (m, 1H), 4.52- 4.38 (m, 1H), 4.38- 4.27 (m, 1H), 4.23- 4.11 (m, 1H), 4.10- 4.03 (m, 1H), 4.01- 3.87 (m, 1H), 3.27- 3.11 (m, 6H), 2.34- 2.25 (m, 1H), 2.17- |
| | | | | 2.06 (m, 2H), 2.04- |
| | | | | 1.96 (m, 2H), 1.95- |
| | | | | 1.87 (m, 3H), 1.86- |
| | | | | 1.76 (m, 2H) |
| 711 | ((S)-(2- (((3S,6S,9aS)-3-(3- (6-(dimethylamino) pyrimidin-4- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 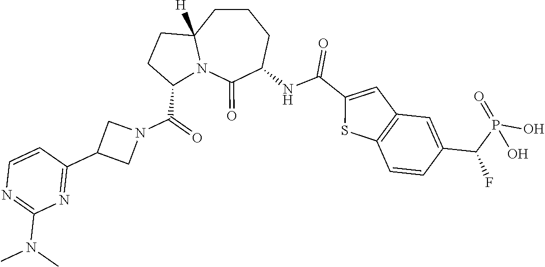 [SMILES: CN(C)c1nccc(C2CN(C(=O)[C@@H]3CC[C@@H]4CCC[C@H](NC(=O)c5cc6cc([C@@H](F)P(=O)(O)O)ccc6s5)C(=O)N43)C2)n1] | 645 | ¹H NMR (400 MHz, DMSO-d₆) δ 8.74 (d, J = 4.2 Hz, 1H), 8.52- 8.43 (m, 1H), 8.24 (d, J = 6.8 Hz, 1H), 8.02 (d, J = 8.8 Hz, 1H), 7.97 (s, 1H), 7.51 (d, J = 7.8 Hz, 1H), 6.65-6.57 (m, 1H), 5.86-5.66 (m, 1H), 4.64 (t, J = 8.4 Hz, 1H), 4.53-4.47 (m, 1H), 4.44 (d, J = 7.3 Hz, 1H), 4.34- 4.26 (m, 1H), 4.19- 4.09 (m, 2H), 4.01- |
| | | | | 3.96 (m, 2H), 3.83- |
| | | | | 3.77 (m, 2H), 3.05 (s, |
| | | | | 3H), 2.98 (s, 3H), |
| | | | | 2.23-2.14 (m, 1H), |
| | | | | 2.04-1.92 (m, 2H), |
| | | | | 1.89-1.76 (m, 4H), |
| | | | | 1.75-1.64 (m, 3H) |
| 712 | ((S)-fluoro(2- (((3S,6S,9aS)-3-(3-(2- morpholinopyridin- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 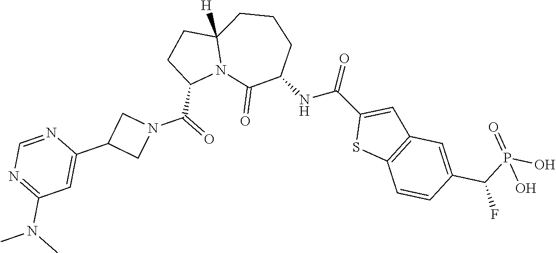 [SMILES: CN(C)c1cc(C2CN(C(=O)[C@@H]3CC[C@@H]4CCC[C@H](NC(=O)c5cc6cc([C@@H](F)P(=O)(O)O)ccc6s5)C(=O)N43)C2)ncn1] | 686.2 | ¹H NMR (400 MHz, CD₃OD) δ 8.12 (s, 1H), 8.06-8.01 (m, 1H), 7.93 (d, J = 8.4 Hz, 1H), 7.70-7.58 (m, 2H), 7.32 (d, J = 6.4 Hz, 1H), 7.18- 7.11 (m, 1H), 5.92- 5.70 (m, 1H), 4.82- 4.55 (m, 3H), 4.54- 4.30 (m, 2H), 4.12- 3.91 (m, 3H), 3.87- 3.71 (m, 4H), 3.68- 3.55 (m, 4H), 2.37- 2.24 (m, 1H), 2.17- |
| | | | | 2.07 (m, 2H), 2.06- |
| | | | | 1.88 (m, 5H), 1.87- |
| | | | | 1.76 (m, 2H) |
| 713 | ((R)-(2- (((3S,6S,9aS)-3-(3- (4-chloro-5- (dimethylamino) pyridin-3-yl)azetidine- 1-carbonyl)-5- oxooctahydro-1H- pyrrolo[1,2- a]azepin-6- yl)carbamoyl)benzo [b]thiophen-5- yl)fluoromethyl) phosphonic acid | 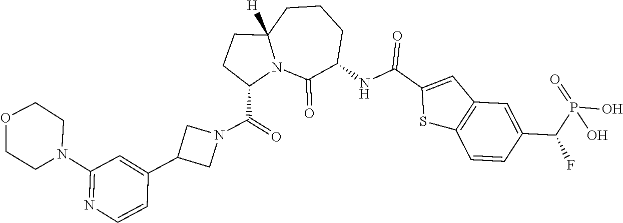 [SMILES: O=C(N[C@H]1CCC[C@H]2CC[C@@H](C(=O)N3CC(c4ccnc(N5CCOCC5)c4)C3)N2C1=O)c1cc2cc([C@@H](F)P(=O)(O)O)ccc2s1] | 678.2 | ¹H NMR (400 MHz, DMSO-d₆) δ 8.78 (t, J = 6.8 Hz, 1H), 8.42- 8.31 (m, 2H), 8.25 (d, J = 20.0 Hz, 1H), 8.07- 7.94 (m, 2H), 7.53 (dd, J = 4.0, 8.4 Hz, 1H), 5.91-5.76 (m, 1H), 4.81 (t, J = 8.8 Hz, 1H), 4.66 (t, J = 8.4 Hz, 1H), 4.48- 4.41 (m, 2H), 4.31 (s, 1H), 4.24-4.12 (m, 2H), 4.10-4.05 (m, |
| | | | | 1H), 2.80 (s, 3H), |
| | | | | 2.78 (s, 3H), 2.24- |
| | | | | 2.13 (m, 1H), 2.09- |
| | | | | 1.91 (m, 2H), 1.83 (d, |
| | | | | J = 11.2 Hz, 2H), 1.80 |
| | | | | (d, J = 6.0 Hz, 2H), |
| | | | | 1.75-1.62 (m, 3H) |
| 714 | (fluoro(2- (((3S,6S,9aS)-3-(3- (3-methoxy-1- methyl-1H-pyrazol- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 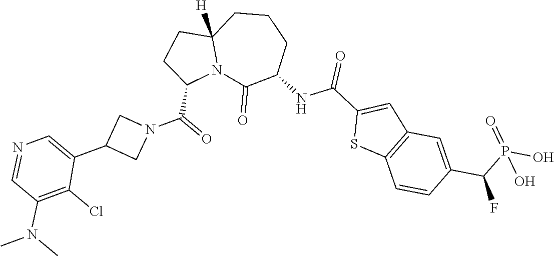 [SMILES: CN(C)c1cncc(C2CN(C(=O)[C@@H]3CC[C@@H]4CCC[C@H](NC(=O)c5cc6cc([C@H](F)P(=O)(O)O)ccc6s5)C(=O)N43)C2)c1Cl] | 634.3 | ¹H NMR (400 MHz, CD₃OD) δ 8.09 (d, J = 2.4 Hz, 1H), 8.05- 8.00 (m, 1H), 7.96 (d, J = 8.4 Hz, 1H), 7.59 (d, J = 8.4 Hz, 1H), 7.49-7.33 (m, 1H), 5.92-5.74 (m, 1H), 4.73 (s, 1H), 4.60- 4.47 (m, 2H), 4.46- 4.30 (m, 1H), 4.29- 4.19 (m, 1H), 4.10- 4.01 (m, 1H), 3.99- 3.91 (m, 1H), 3.86 (d, J = 11.6 Hz, 3H), 3.79- 3.71 (m, 1H), 3.70- |
| | | | | 3.62 (m, 3H), 2.36- |
| | | | | 2.25 (m, 1H), 2.16- |
| | | | | 2.05 (m, 2H), 2.04- |
| | | | | 1.98 (m, 1H), 1.98- |
| | | | | 1.86 (m, 4H), 1.85- |
| | | | | 1.76 (m, 2H) |
| 715 | (fluoro(2- (((3S,6S,9aS)-3-(3- (3-fluoro-1-methyl- 1H-pyrazol-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 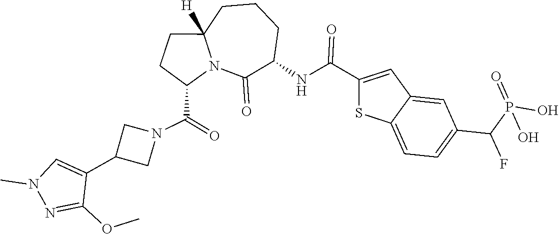 [SMILES: COc1nn(C)cc1C1CN(C(=O)[C@@H]2CC[C@@H]3CCC[C@H](NC(=O)c4cc5cc(C(F)P(=O)(O)O)ccc5s4)C(=O)N32)C1] | 622.1 | ¹H NMR (400 MHz, DMSO-d₆) δ 8.86- 8.67 (m, 1H), 8.26 (d, J = 2.8 Hz, 1H), 8.03 (d, J = 8.4 Hz, 1H), 7.97 (d, J = 3.6 Hz, 1H), 7.76-7.66 (m, 1H), 7.53 (d, J = 8.4 Hz, 1H), 5.97-5.69 (m, 1H), 4.73-4.60 (m, 1H), 4.53-4.37 (m, 1H), 4.25-4.15 (m, 1H), 4.10-4.05 (m, 1H), 4.03-3.94 |
| | | | | (m, 2H), 3.68 (s, 3H), |
| | | | | 3.61 (d, J = 2.0 Hz, |
| | | | | 2H), 2.26-2.11 (m, |
| | | | | 1H), 2.06-1.92 (m, |
| | | | | 2H), 1.92-1.74 (m, |
| | | | | 4H), 1.71-1.69 (m, |
| | | | | 2H), 1.65 (s, 1H) |
| 716 | (fluoro(2- (((3S,6S,9aS)-3-(3- (1-methyl-1H- pyrazol-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 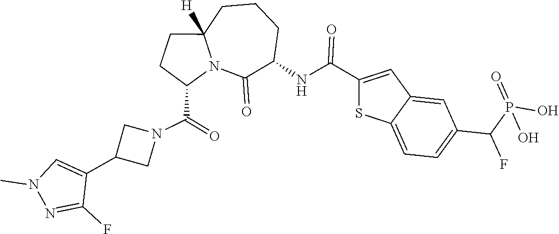 [SMILES: Cn1cc(C2CN(C(=O)[C@@H]3CC[C@@H]4CCC[C@H](NC(=O)c5cc6cc(C(F)P(=O)(O)O)ccc6s5)C(=O)N43)C2)c(F)n1] | 604.2 | ¹H NMR (400 MHz, DMSO-d₆) δ 8.70 (d, J = 7.6 Hz, 1H), 8.24 (s, 1H), 8.15 (t, J = 5.6 Hz, 1H), 8.03 (d, J = 8.4 Hz, 1H), 7.97 (s, 1H), 7.76 (s, 1H), 7.53 (d, J = 8.4 Hz, 1H), 7.45 (s, 1H), 6.25 (d, J = 11.6 Hz, 1H), 5.91-5.75 (m, 1H), 5.40-5.31 (m, 1H), 4.63 (t, J = 8.4 Hz, 1H), 4.43 (dd, J = 3.6, 8.0 Hz, 1H), 4.03- 3.91 (m, 2H), 3.88 |
| | | | | (t, J = 5.6 Hz, 2H), |
| | | | | 3.82 (s, 3H), 2.23- |
| | | | | 2.12 (m, 1H), 2.01- |
| | | | | 1.90 (m, 2H), 1.89- |
| | | | | 1.80 (m, 2H), 1.78- |
| | | | | 1.65 (m, 5H) |
| 717 | ((2-(((3S,6S,9aS)-3- (3-(5-chloro-4- fluoropyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 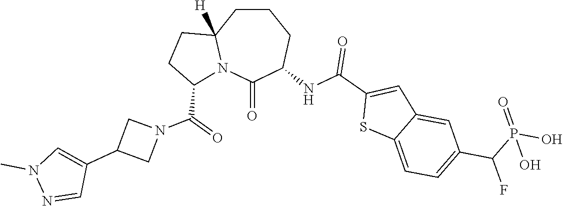 [SMILES: Cn1cc(C2CN(C(=O)[C@@H]3CC[C@@H]4CCC[C@H](NC(=O)c5cc6cc(C(F)P(=O)(O)O)ccc6s5)C(=O)N43)C2)cn1] | 653.2 | ¹H NMR (400 MHz, DMSO-d₆) δ 8.77 (s, 1H), 8.71-8.63 (m, 2H), 8.19 (d, J = 10.8 Hz, 1H), 7.92-7.85 (m, 2H), 7.58-7.48 (m, 1H), 5.57-5.27 (m, 2H), 4.79 (t, J = 8.8 Hz, 1H), 4.62 (d, J = 4.8 Hz, 1H), 4.60- 4.50 (m, 1H), 4.44 (dd, J = 4.4, 7.6 Hz, 1H), 4.42-4.36 (m, |
| | | | | 1H), 4.31-4.24 (m, |
| | | | | 1H), 4.16-4.11 (m, |
| | | | | 1H), 4.07 (d, J = 8.0 |
| | | | | Hz, 1H), 4.02-3.96 |
| | | | | (m, 1H), 2.24-2.15 |
| | | | | (m, 1H), 2.08-1.92 |
| | | | | (m, 2H), 1.90-1.76 |
| | | | | (m, 4H), 1.72 (s, 3H) |
| 718 | (difluoro(2- (((3S,6S,10aS)-5- oxo-3-(3-(pyridin-2- ylmethyl)azetidine-1- carbonyl)decahydro pyrrolo[1,2- a]azocin-6- yl)carbamoyl)benzo [b]thiophen-5- yl)methyl) phosphonic acid | 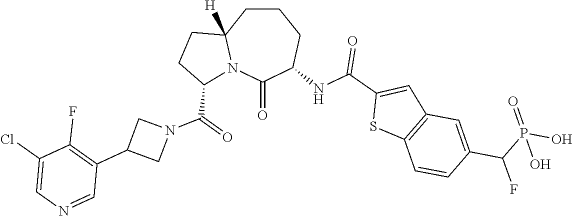 [SMILES: O=C(N[C@H]1CCC[C@H]2CC[C@@H](C(=O)N3CC(c4cncc(Cl)c4F)C3)N2C1=O)c1cc2cc(C(F)P(=O)(O)O)ccc2s1] | 646.9 | ¹H NMR (400 MHz, CD₃OD) δ 8.66-8.55 (m, 1H), 8.47-8.32 (m, 1H), 8.16-8.06 (m, 2H), 7.97-7.63 (m, 4H), 5.00-4.96 (m, 1H), 4.47-4.24 (m, 3H), 4.24-3.97 (m, 1H), 3.88-3.70 (m, 1H), 3.38-3.31 (m, 3H), 3.17-3.08 (m, 1H), 2.28-2.14 (m, 2H), 2.07-1.88 |
| | | | | (m, 6H), 1.88-1.75 |
| | | | | (m, 2H), 1.73-1.55 |
| | | | | (m, 2H) |
| 719 | (difluoro(2- (((3S,6S,10aS)-3-(3- (5-(S- methylsulfonimidoyl) pyrimidin-2- yl)azetidine-1- carbonyl)-5- oxodecahydropyrrolo [1,2-a]azocin-6- yl)carbamoyl)benzo [b]thiophen-5- yl)methyl) phosphonic acid | 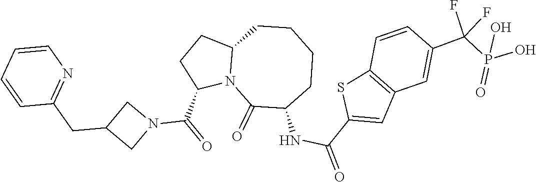 [SMILES: O=C(N[C@H]1CCCC[C@H]2CC[C@@H](C(=O)N3CC(Cc4ccccn4)C3)N2C1=O)c1cc2cc(C(F)(F)P(=O)(O)O)ccc2s1] | 711.2 | 1H NMR (400 MHz, CD₃OD) δ 9.25 (s, 1H), 9.18 (d, J = 4.4 Hz, 1H), 8.20-8.14 (m, 1H), 8.14-8.01 (m, 1H), 7.96-7.84 (m, 1H), 7.77-7.70 (m, 1H), 5.07-4.92 (m, 2H), 4.73-4.54 (m, 1H), 4.53-4.44 (m, 2H), 4.44-4.37 (m, 1H), 4.36-4.22 (m, 2H), 3.28-3.20 |
| | | | | (m, 3H), 2.33-2.17 |
| | | | | (m, 2H), 2.09-1.90 |
| | | | | (m, 6H), 1.89-1.76 |
| | | | | (m, 2H), 1.73-1.57 |
| | | | | (m, 2H) |
| 720 | ((2-(((3S,6S,9aS)-5- oxo-3-(3-(pyridin-3- yl)azetidine-1- carbonyl)octahydro- 1H-pyrrolo[1,2- a]azepin-6- yl)carbamoyl)-1H- indol-5- yl)methyl) phosphonic acid | 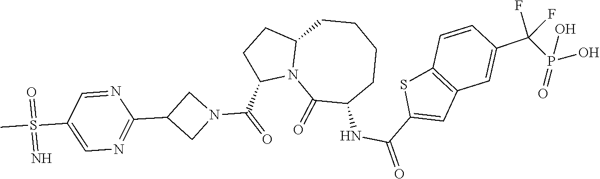 [SMILES: CS(=N)(=O)c1cnc(C2CN(C(=O)[C@@H]3CC[C@@H]4CCCC[C@H](NC(=O)c5cc6cc(C(F)(F)P(=O)(O)O)ccc6s5)C(=O)N43)C2)nc1] | 566.1 | ¹H NMR (400 MHz, CD₃OD) δ 8.71- 8.60 (m, 2H), 8.59- 8.46 (m, 1H), 7.92- 7.72 (m, 1H), 7.56 (s, 1H), 7.40-7.33 (m, 1H), 7.25-7.18 (m, 1H), 7.10 (s, 1H), 4.79 (d, J = 10.4 Hz, 1H), 4.70 (t, J = 8.4 Hz, 1H), 4.61-4.47 |
| | | | | (m, 2H), 4.46-4.25 |
| | | | | (m, 1H), 4.15-3.99 |
| | | | | (m, 3H), 3.23-3.15 |
| | | | | (m, 2H), 2.37-2.27 |
| | | | | (m, 1H), 2.21-2.07 |
| | | | | (m, 2H), 2.06-1.92 |
| | | | | (m, 4H), 1.90-1.77 |
| | | | | (m, 3H) |
| 721 | (difluoro(2- (((3S,6S,9aS)-3-(3- methoxy-3-(4- methylpyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 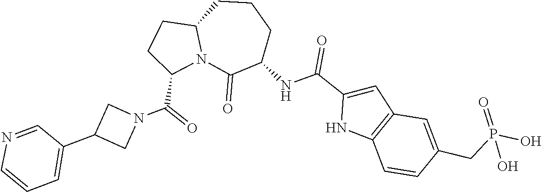 | 664.1 | ¹H NMR (400 MHz, CD₃OD) δ 9.03-8.94 (m, 1H), 8.76 (m, 1H), 8.17-8.13 (m, 1H), 8.13-8.03 (m, 1H), 8.02-7.95 (m, 1H), 7.68 (m, 1H), 5.04-4.98 (m, 1H), 4.81 (m, 1H), 4.79- 4.69 (m, 1H), 4.65- 4.56 (m, 1H), 4.55- 4.44 (m, 1H), 4.36- 4.23 (m, 1H), 4.07 (m, 1H), 3.08-3.02 |
| | | | | (m, 3H), 2.52 (m, |
| | | | | 3H), 2.38-2.26 (m, |
| | | | | 1H), 2.23-2.13 (m, |
| | | | | 1H), 2.12-2.02 (m, |
| | | | | 2H), 2.02-1.81 (m, |
| | | | | 6H) |
| 722 | (difluoro(2- (((3S,6S,9aS)-3-(3- methoxy-3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 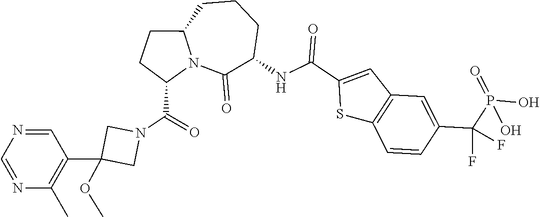 [SMILES: COC1(c2cncnc2C)CN(C(=O)[C@@H]2CC[C@@H]3CCC[C@H](NC(=O)c4cc5cc(C(F)(F)P(=O)(O)O)ccc5s4)C(=O)N32)C1] | 663.0 | ¹H NMR (400 MHz, CD₃OD) δ 8.64-8.57 (m, 1H), 8.40-8.34 (m, 1H), 8.22 (d, J = 5.6 Hz, 1H), 8.06 (s, 1H), 7.98 (s, 1H), 7.90-7.86 (m, 1H), 7.86-7.75 (m, 1H), 5.00 (d, J = 9.6 Hz, 1H), 4.72 (d, J = 10.4 Hz, 1H), 4.62 (d, J = 10.0 Hz, 1H), 4.56- 4.44 (m, 2H), 4.35- |
| | | | | 4.24 (m, 1H), 4.09 (s, |
| | | | | 1H), 3.04-2.94 (m, |
| | | | | 3H), 2.61-2.48 (m, |
| | | | | 3H), 2.38-2.27 (m, |
| | | | | 1H), 2.22-2.14 (m, |
| | | | | 1H), 2.12-2.06 (m, |
| | | | | 1H), 2.05-1.95 (m, |
| | | | | 3H), 1.93-1.75 (m, |
| | | | | 4H) |
| 723 | (difluoro(2- (((3S,6S,9aS)-3-(3- methoxy-3- (pyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 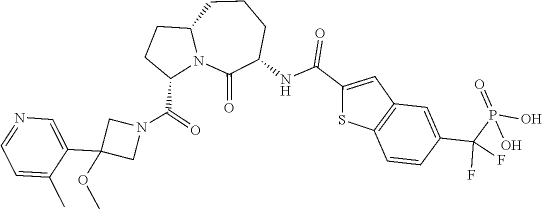 [SMILES: COC1(c2cnccc2C)CN(C(=O)[C@@H]2CC[C@@H]3CCC[C@H](NC(=O)c4cc5cc(C(F)(F)P(=O)(O)O)ccc5s4)C(=O)N32)C1] | 650.1 | ¹H NMR (400 MHz, CD₃OD) δ 9.17 (s, 1H), 9.00 (s, 1H), 8.95 (s, 1H), 8.22 (s, 1H), 8.14-8.09 (m, 1H), 7.93 (d, J = 8.4 Hz, 1H), 7.76 (d, J = 8.4 Hz, 1H), 4.93- 4.89 (m, 1H), 4.78- 4.71 (m, 1H), 4.63- 4.55 (m, 2H), 4.39- 4.24 (m, 2H), 4.07 (d, J = 5.2 Hz, 1H), 3.26 |
| | | | | -3.21 (m, 3H), 2.38- |
| | | | | 2.25 (m, 1H), 2.16- |
| | | | | 1.93 (m, 6H), 1.87- |
| | | | | 1.76 (m, 3H) |
| 724 | (difluoro(2- (((3S,6S,10aS)-5- oxo-3-(3-(pyridin-3- yloxy)azetidine-1- carbonyl)decahydro pyrrolo[1,2- a]azocin-6- yl)carbamoyl)benzo [b]thiophen-5- yl)methyl) phosphonic acid | 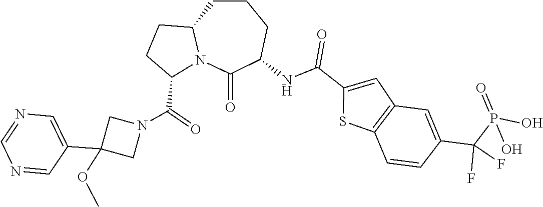 [SMILES: COC1(c2cncnc2)CN(C(=O)[C@@H]2CC[C@@H]3CCC[C@H](NC(=O)c4cc5cc(C(F)(F)P(=O)(O)O)ccc5s4)C(=O)N32)C1] | 649.2 | ¹H NMR (400 MHz, CD₃OD) δ 8.25-8.05 (m, 4H), 7.98-7.86 (m, 1H), 7.80-7.68 (m, 1H), 7.43-7.26 (m, 2H), 5.15 (d, J = 4.0 Hz, 1H), 5.00 (m, 2H), 4.76-4.56 (m, 1H), 4.55-4.35 (m, 3H), 4.12-3.91 (m, 1H), 2.35-2.15 (m, 2H), 1.94 (s, 6H), 1.89-1.75 (m, 2H), 1.74-1.57 (m, 2H) |
| 725 | (difluoro(2- (((3S,6S,10aS)-3- (((S)-1-hydroxy-3- (pyridin-3- yl)propan-2- yl)carbamoyl)-5- oxodecahydropyrrolo [1,2-a]azocin-6- yl)carbamoyl)benzo [b]thiophen-5- yl)methyl) phosphonic acid | 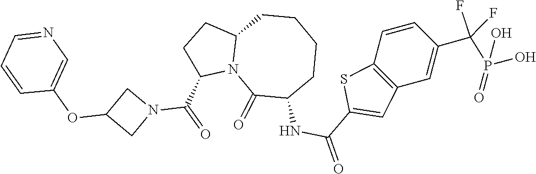 [SMILES: O=C(N[C@H]1CCCC[C@H]2CC[C@@H](C(=O)N3CC(Oc4cccnc4)C3)N2C1=O)c1cc2cc(C(F)(F)P(=O)(O)O)ccc2s1] | 651.1 | ¹H NMR (400 MHz, CD₃OD) δ 8.63 (d, J = 1.6 Hz, 1H), 8.51 (d, J = 4.4 Hz, 1H), 8.19-8.07 (m, 3H), 7.98 (s, 1H), 7.79- 7.68 (m, 2H), 5.11 (t, J = 8.8 Hz, 1H), 4.41- 4.27 (m, 2H), 4.15- 4.05 (m, 1H), 3.57 (d, J = 5.6 Hz, 2H), 3.09 (m, 1H), 2.84 (m, |
| | | | | 1H), 2.24-2.09 (m, |
| | | | | 2H), 2.04-1.83 (m, |
| | | | | 5H), 1.81-1.74 (m, |
| | | | | 1H), 1.73-1.51 (m, |
| | | | | 4H) |
| 726 | ((2-(((3S,6S,10aS)- 3-(((212-pyrazol-3- yl)methyl) carbamoyl)-5- oxodecahydropyrrolo [1,2-a]azocin-6- yl)carbamoyl)benzo [b]thiophen-5- yl)difluoromethyl) phosphonic acid | 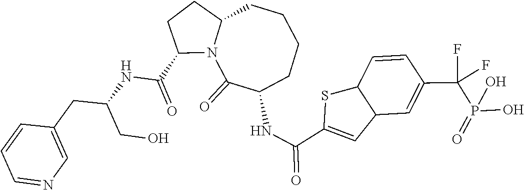 [SMILES: O=C(N[C@H]1CCCC[C@H]2CC[C@@H](C(=O)N[C@H](CO)Cc3cccnc3)N2C1=O)C1=CC2C=C(C(F)(F)P(=O)(O)O)C=CC2S1] | 596.1 | ¹H NMR (400 MHz, DMSO-d₆) δ 8.89 (d, J = 6.8 Hz, 1H), 8.41- 8.29 (m, 2H), 8.16- 8.06 (m, 2H), 7.62- 7.48 (m, 2H), 7.01- 6.89 (m, 1H), 6.17 (d, J = 2.0 Hz, 1H), 4.99- 4.89 (m, 1H), 4.38- 4.32 (m, 1H), 4.31- 4.21 (m, 3H), 2.16- 2.01 (m, 2H), 2.01- |
| | | | | 1.95 (m, 1H), 1.95- |
| | | | | 1.75 (m, 5H), 1.75- |
| | | | | 1.63 (m, 2H), 1.62- |
| | | | | 1.47 (m, 2H) |
| 727 | (difluoro(2- (((3S,6S,10aS)-3- (((R)-1-hydroxy-3- (pyridin-3- yl)propan-2- yl)carbamoyl)-5- oxodecahydropyrrolo [1,2-a]azocin-6- yl)carbamoyl)benzo [b]thiophen-5- yl)methyl) phosphonic acid | 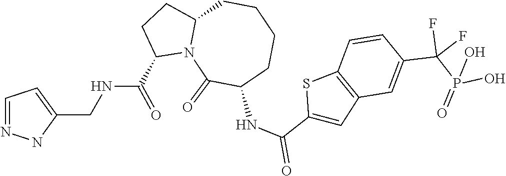 [SMILES: O=C(N[C@H]1CCCC[C@H]2CC[C@@H](C(=O)NCc3ccn[nH]3)N2C1=O)c1cc2cc(C(F)(F)P(=O)(O)O)ccc2s1] | 651.1 | ¹H NMR (400 MHz, DMSO-d₆) δ 8.85 (d, J = 8.0 Hz, 1 H) 8.47 (s, 2H) 8.31 (s, 1H) 8.13 (d, J = 8.0 Hz, 1H) 8.06 (s, 1H) 7.89 (d, J = 8.0 Hz, 1H) 7.73-7.79 (m, 1H) 7.58 (d, J = 8.0Hz, 1H) 7.41-7.46 (m, 1H) 4.86-4.92 (m, 1H) 4.20 (m, 2H) 3.90- 3.99 (m, 2H) 3.48 (s, |
| | | | | 1H) 2.98 (m, 2H) |
| | | | | 1.76-1.94 (m, 6H) |
| | | | | 1.55-1.65 (m, 3H) |
| | | | | 1.41-1.51 (m, 3H) |
| | | | | 1.24 (m, 1H) |
| 728 | (difluoro(2- (((3S,6S,8aS)-5- oxo-3-(3-(pyridin-2- yl)azetidine-1- carbonyl)octahydroi ndolizin-6- yl)carbamoyl)benzo [b]thiophen-5- yl)methyl) phosphonic acid | 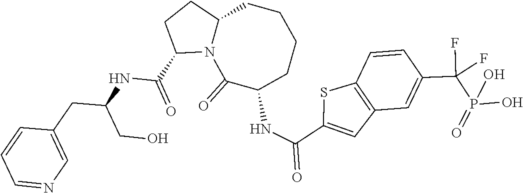 [SMILES: O=C(N[C@H]1CCCC[C@H]2CC[C@@H](C(=O)N[C@@H](CO)Cc3cccnc3)N2C1=O)c1cc2cc(C(F)(F)P(=O)(O)O)ccc2s1] | 605.2 | ¹H NMR (400 MHz, CD₃OD) δ 8.62-8.47 (m, 1H), 8.21 ( s, 1H), 8.08 (d, J = 5.6 Hz, 1H), 7.93 (d, J = 8.0 Hz, 1H), 7.79 (d, J = 7.2 Hz, 2H), 7.55- 7.35 (m, 1H), 7.34- 7.24 (m, 1H), 4.85- 4.72 (m, 2H), 4.67 (d, J = 7.6 Hz, 1H), 4.48 (d, J = 8.4 Hz, 1H), |
| | | | | 4.44-4.36 (m, 1H), |
| | | | | 4.25-4.03 (m, 2H), |
| | | | | 3.76 (s, 1H), 2.26- |
| | | | | 2.09 (m, 4H), 2.04- |
| | | | | 1.97 (m, 1H), 1.93- |
| | | | | 1.78 (m, 2H), 1.32 (d, |
| | | | | J = 6.4 Hz, 1H) |
| 729 | ((2- (((1R,3S,6S,10aS,Z)- 3-((rel-trans)-3- cyano-4- phenylpyrrolidine- 1-carbonyl)-1- hydroxy-5-oxo- 1,2,3,5,6,7,10,10a- octahydropyrrolo[1, 2-a]azocin-6- yl)carbamoyl)benzo [b]thiophen-5- | 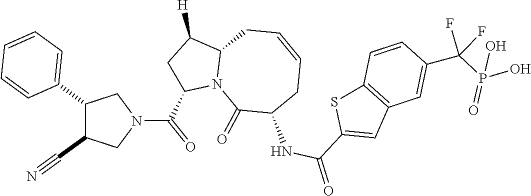 | 685.2 | ¹H NMR (400 MHz, CD₃OD) δ 8.26-8.09 (m, 2H), 8.06-7.95 (m, 1H), 7.75-7.64 (m, 1H), 7.49-7.26 (m, 5H), 5.87 (d, J = 4.4 Hz, 2H), 5.22- 5.14 (m, 1H), 4.85- 4.77 (m, 1H), 4.33- 4.26 (m, 1H), 4.24- 3.93 (m, 3H), 3.91- 3.74 (m, 1H), 3.70- |
| | yl)difluoromethyl) | | | 3.56 (m, 1H), 3.52 (s, |
| | phosphonic acid | | | 1H), 2.86-2.55 (m, |
| | | | | 4H), 2.28-2.08 (m, |
| | | | | 2H) |
| 730 | (difluoro(2- (((1R,3S,6S,10aR,Z)- 1-hydroxy-3-(3- (morpholine-4- carbonyl)azetidine- 1-carbonyl)-5-oxo- 1,2,3,5,6,7,10,10a- octahydropyrrolo[1, 2-a]azocin-6- yl)carbamoyl)benzo [b]thiophen-5- yl)methyl) phosphonic acid | 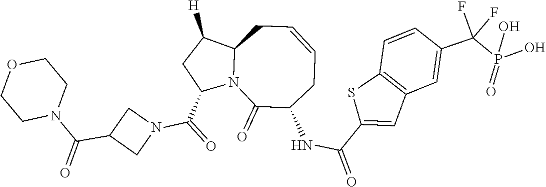 | 683.1 | ¹H NMR (400 MHz, CD₃OD) δ 8.25 (s, 1H), 8.07 (d, J = 7.6 Hz, 1H), 7.90-7.83 (m, 2H), 5.90-5.62 (m, 2H), 5.10-4.94 (m, 2H), 4.78-4.57 (m, 3H), 4.45-4.32 (m, 2H), 4.25-4.00 (m, 2H), 3.85-3.75 (m, 1H), 3.68-3.57 (m, 6H), 3.39-3.36 (m, 1H), 3.06-2.94 |
| | | | | (m, 1H), 2.67-2.57 |
| | | | | (m, 1H), 2.51-2.31 |
| | | | | (m, 2H), 2.22-2.12 |
| | | | | (m, 1H), 2.05-1.94 |
| | | | | (m, 1H) |
| 731 | (difluoro(2- (((3S,6S,8aS)-3-(3- (morpholine-4- carbonyl)azetidine- 1-carbonyl)-5- oxooctahydro- indolizin-6- yl)carbamoyl)benzo [b]thiophen-5- yl)methyl) phosphonic acid | 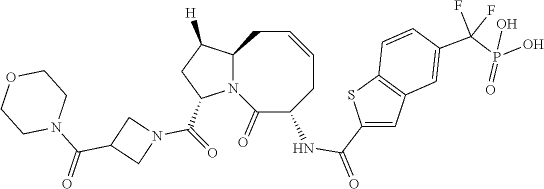 | 641.1 | ¹H NMR (400 MHz, CD₃OD) δ 8.17-8.07 (m, 2H), 8.03 (d, J = 8.4 Hz, 1H), 7.68 (d, J = 8.4 Hz, 1H), 4.78- 4.59 (m, 2H), 4.48- 4.40 (m, 2H), 4.28- 4.04 (m, 2H), 3.85- 3.71 (m, 2H), 3.69- 3.54 (m, 6H), 3.42- 3.32 (m, 2H), 2.31- 2.09 (m, 4H), 2.03- |
| | | | | 1.93 (m, 2H), 1.91- |
| | | | | 1.74 (m, 2H) |
| 732 | ((2-(((3S,6S,8aS)-3- (3-(1H-imidazol-1- yl)azetidine-1- carbonyl)-5- oxooctahydro- indolizin-6- yl)carbamoyl)benzo [b]thiophen-5- yl)difluoromethyl) phosphonic acid | 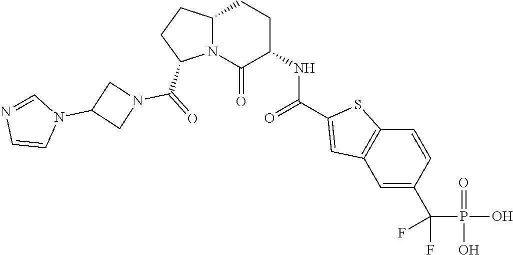 [SMILES: O=C(N[C@H]1CC[C@H]2CC[C@@H](C(=O)N3CC(n4ccnc4)C3)N2C1=O)c1cc2cc(C(F)(F)P(=O)(O)O)ccc2s1] | 594.3 | ¹H NMR (400 MHz, CD₃OD) δ 8.48 (s, 1H), 8.22-8.17 (m, 1H), 8.12-8.06 (m, 1H), 7.97 (d, J = 8.0 Hz, 1H), 7.84-7.61 (m, 2H), 7.32-7.21 (m, 1H), 5.38-5.28 (m, 1H), 4.86-4.77 (m, 3H), 4.65-4.51 (m, 1H), 4.50-4.45 (m, 1H), 4.28-4.17 (m, 1H), 3.85-3.75 (m, 1H), 2.36-2.13 (m, 4H), 2.12-1.98 |
| | | | | (m, 2H), 1.97-1.78 |
| | | | | (m, 2H) |
| 733 | ((2-(((3S,6S,8aS)-3- ((rel-trans)-3- cyano-4- phenylpyrrolidine- 1-carbonyl)-5- oxooctahydro- indolizin-6- yl)carbamoyl)benzo [b]thiophen-5- yl)difluoromethyl) phosphonic acid | 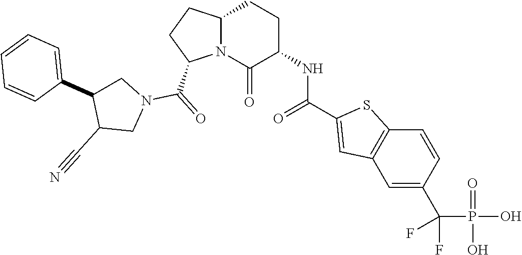 [SMILES: N#CC1CN(C(=O)[C@@H]2CC[C@@H]3CC[C@H](NC(=O)c4cc5cc(C(F)(F)P(=O)(O)O)ccc5s4)C(=O)N32)C[C@@H]1c1ccccc1] | 643.1 | ¹H NMR (400 MHz, CD₃OD) δ 8.14 (d, J = 7.2 Hz, 1H), 8.12- 8.08 (m, 1H), 8.03 (d, J = 8.4 Hz, 1H), 7.68 (d, J = 8.8 Hz, 1H), 7.49-7.37 (m, 4H), 7.36-7.28 (m, 1H), 4.82-4.75 (m, 1H), 4.74-4.62 (m, 1H), 4.49-4.12 (m, 1H), 4.13-3.95 (m, 1H), 3.90-3.75 (m, 2H), 3.74-3.39 (m, 3H), 2.36-2.11 (m, 4H), |
| | | | | 2.09-1.96 (m, 2H), |
| | | | | 1.92-1.78 (m, 2H) |
| 734 | ((2-(((3S,6S,9aS)-3- (3-(3-cyano-1- methyl-1H-pyrazol- 4-yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 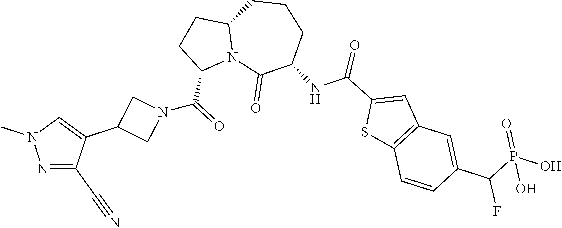 [SMILES: Cn1cc(C2CN(C(=O)[C@@H]3CC[C@@H]4CCC[C@H](NC(=O)c5cc6cc(C(F)P(=O)(O)O)ccc6s5)C(=O)N43)C2)c(C#N)n1] | 629.2 | ¹H NMR (400 MHz, CD₃OD) δ 8.10 (d, J = 6.0 Hz, 1H), 8.07- 7.81 (m, 3H), 7.62- 7.57 (m, 1H), 5.90- 5.75 (m, 1H), 4.78- 4.73 (m, 1H), 4.69 (t, J = 8.4 Hz, 1H), 4.60- 4.46 (m, 2H), 4.42- 4.25 (m, 1H), 4.13- 4.04 (m, 1H), 4.03- 3.92 (m, 4H), 3.85 (d, J = 2.4 Hz, 1H), 2.37- 2.25 (m, 1H), 2.13- |
| | | | | 2.06 (m, 2H), 2.06- |
| | | | | 1.99 (m, 2H), 1.98- |
| | | | | 1.88 (m, 3H), 1.88- |
| | | | | 1.81 (m, 2H) |
| 735 | ((R)-(2- (((3S,6S,8S,9aR)-3- (3-(4- (dimethylamino) pyridin-3- yl)azetidine- 1-carbonyl)-8- methyl-5- oxooctahydro-1H- pyrrolo[1,2- a]azepin-6- yl)carbamoyl)benzo [b]thiophen-5- yl)fluoromethyl) | 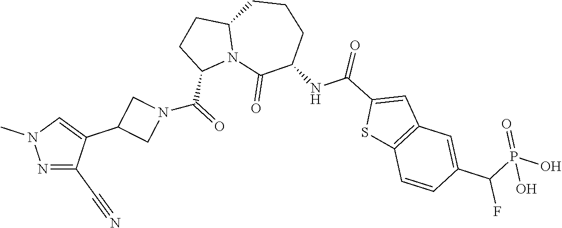 [SMILES: Cn1cc(C2CN(C(=O)[C@@H]3CC[C@@H]4CCC[C@H](NC(=O)c5cc6cc(C(F)P(=O)(O)O)ccc6s5)C(=O)N43)C2)c(C#N)n1] | 658.3 | ¹H NMR (400 MHz, CD₃OD) δ 8.16-7.92 (m, 3H), 7.83-7.51 (m, 3H), 6.94-6.70 (m, 1H), 5.82-5.77 (m, 1H), 5.01-4.98 (m, 2H), 4.64-4.40 (m, 3H), 4.29-4.19 (m, 2H), 3.96-3.95 (m, 1H), 3.05-2.98 (m, 6H), 2.33-2.27 (m, 3H), 2.04-1.82 (m, 5H), 1.62-1.60 (m, 1H), 1.24-1.21 |
| | phosphonic acid | | | (m, 3H). |
| 736 | ((R)-(2- (((3S,6S,8R,9aR)-3- (3-(4- (dimethylamino) pyridin-3- yl)azetidine- 1-carbonyl)-8- methyl-5- oxooctahydro-1H- pyrrolo[1,2- a]azepin-6- yl)carbamoyl)benzo [b]thiophen-5- yl)fluoromethyl) phosphonic acid | 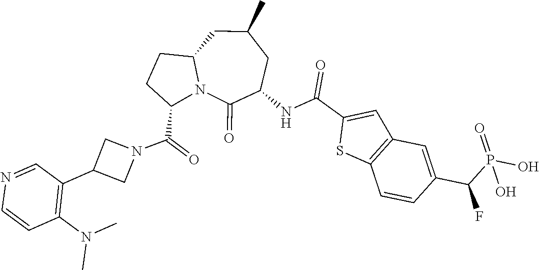 [SMILES: C[C@@H]1C[C@H]2CC[C@@H](C(=O)N3CC(c4cnccc4N(C)C)C3)N2C(=O)[C@@H](NC(=O)c2cc3cc([C@H](F)P(=O)(O)O)ccc3s2)C1] | 658.3 | ¹H NMR (400 MHz, CD₃OD) δ 8.22-8.15 (m, 1H), 8.02-7.95 (m, 2H), 7.86-7.78 (m, 1H), 7.58-7.57 (m, 1H), 7.47-7.35 (m, 1H), 7.00-6.86 (m, 1H), 5.77-5.63 (m, 1H), 4.78-4.73 (m, 1H), 4.70-4.67 (m, 1H), 4.53-4.49 (m, 2H), 4.43-4.37 (m, 1H), 4.27-4.22 (m, 1H), 4.09-4.08 (m, 2H), 3.13-3.05 (m, 6H), 2.37-2.28 |
| | | | | (m, 1H), 2.09-2.05 |
| | | | | (m, 2H), 1.97-1.94 |
| | | | | (m, 3H), 1.82-1.77 |
| | | | | (m, 1H), 1.70-1.67 |
| | | | | (m, 2H), 1.05 (d, J = |
| | | | | 6.0 Hz, 3H) |
| 737 | (fluoro(2- (((3S,6S,9aS)-3- ((2R,3S)-2-methyl- 3-(pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid Or (fluoro(2- (((3S,6S,9aS)-3- ((2S,3R)-2-methyl- | 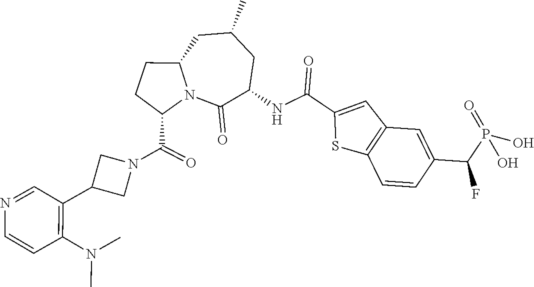 [SMILES: C[C@H]1C[C@H]2CC[C@@H](C(=O)N3CC(c4cnccc4N(C)C)C3)N2C(=O)[C@@H](NC(=O)c2cc3cc([C@H](F)P(=O)(O)O)ccc3s2)C1] | 615.2 | ¹H NMR (400 MHz, CD₃OD) δ 8.76 (d, J = 8.0 Hz, 1H), 8.71 (s, 1H), 8.48 (d, J = 6.0 Hz, 1H), 8.06- 7.98 (m, 2H), 7.93 (d, J = 8.8 Hz, 1H), 7.88 (t, J = 7.6 Hz, 1H), 7.69-7.53 (m, 1H), 5.96-5.70 (m, 1H), 4.87-4.78 (m, 1H), 4.71 (d, J = 11.2 Hz, 1H), 4.58-4.47 (m, 1H), 4.45-4.33 (m, 1H), 4.11 (d, J = 2.0 Hz, 1H), 4.01-3.92 (m, 1H), 3.66-3.64 |
| | 3-(pyridin-3- | | | (m, 1H), 2.46-2.29 |
| | yl)azetidine-1- | Azetidine BB SFC peak 2 was | | (m, 1H), 2.25-1.99 |
| | carbonyl)-5- | used for synthesis and biological | | (m, 5H), 1.98-1.78 |
| | oxooctahydro-1H- | testing | | (m, 4H), 1.70 (d, J = |
| | pyrrolo[1,2- | | | 6.1 Hz, 3H) |
| | a]azepin-6- | | | |
| | yl)carbamoyl)benzo | | | |
| | [b]thiophen-5- | | | |
| | yl)methyl) | | | |
| | phosphonic acid | | | |
| 738 | (fluoro(2- (((3S,6S,9aS)-3- ((2R,3S)-2-methyl- 3-(pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid Or | 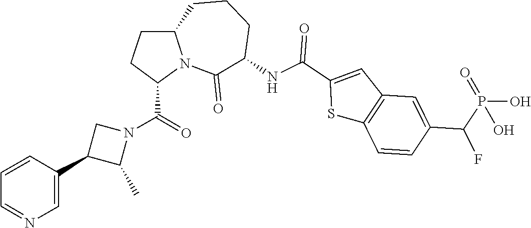 [SMILES: C[C@@H]1[C@@H](c2cccnc2)CN1C(=O)[C@@H]1CC[C@@H]2CCC[C@H](NC(=O)c3cc4cc(C(F)P(=O)(O)O)ccc4s3)C(=O)N21] | 615.3 | ¹H NMR (400 MHz, CD₃OD) δ 8.69 (d, J = 8.0 Hz, 1H), 8.60 (d, J = 2.8 Hz, 1H), 8.50 (d, J = 5.2 Hz, 1H), 8.01 (s, 2H), 7.90 (d, J = 8.4 Hz, 1H), 7.77 (dd, J = 5.6, 8.0 Hz, 1H), 7.60 (d, J = 8.4 Hz, 1H), 5.92- 5.65 (m, 1H), 4.75 (d, J = 11.2 Hz, 1H), 4.71-4.63 (m, 1H), 4.53 (dd, J = 6.4, 8.8 |
| | (fluoro(2- | | | Hz, 1H), 4.47 (t, J = |
| | (((3S,6S,9aS)-3- | or | | 6.8 Hz, 1H), 4.41 (t, |
| | ((2S,3R)-2-methyl- | | | J = 6.0 Hz, 1H), 4.10 |
| | 3-(pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 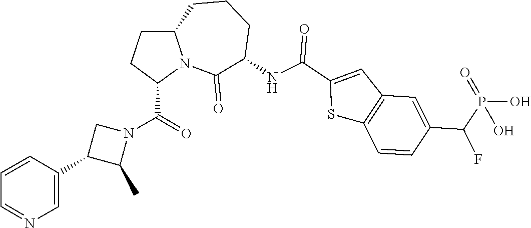 [SMILES: C[C@H]1[C@H](c2cccnc2)CN1C(=O)[C@@H]1CC[C@@H]2CCC[C@H](NC(=O)c3cc4cc(C(F)P(=O)(O)O)ccc4s3)C(=O)N21] | | (d, J = 6.0 Hz, 1H), 3.58 (q, J = 6.4 Hz, 1H), 2.42-2.29 (m, 1H), 2.25-2.09 (m, 2H), 2.09-1.97 (m, 3H), 1.94-1.75 (m, 4H), 1.58 (d, J = 6.3 Hz, 3H) |
| | | Azetidine BB SFC peak 1 was | | |
| | | used for synthesis and biological | | |
| | | testing | | |
| 739 | ((2-(((3S,6S,9aS)-3- (3-(4- (dimethylamino) pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 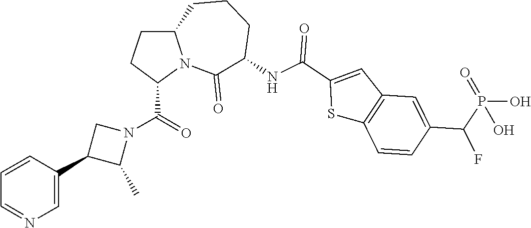 [SMILES: C[C@@H]1[C@@H](c2cccnc2)CN1C(=O)[C@@H]1CC[C@@H]2CCC[C@H](NC(=O)c3cc4cc(C(F)P(=O)(O)O)ccc4s3)C(=O)N21] | 645.2 | ¹H NMR (400 MHz, CD₃OD) δ 8.41-8.27 (m, 1H), 8.18 (d, J = 11.6 Hz, 1H), 8.05- 7.93 (m, 2H), 7.91- 7.84 (m, 1H), 7.66- 7.50 (m, 1H), 5.88- 5.64 (m, 1H), 4.73 (d, J= 10.4 Hz, 1H), 4.65 (t, J = 8.8 Hz, 1H), 4.57-4.46 (m, 2H), 4.42-4.31 (m, 1H), 4.26-4.19 (m, 1H), 4.07 (d, J = 9.6 Hz, |
| | | | | 1H), 4.05-3.96 (m, |
| | | or | | 1H), 3.25 (s, 6H), |
| | | | | 2.37-2.24 (m, 1H), |
| | | 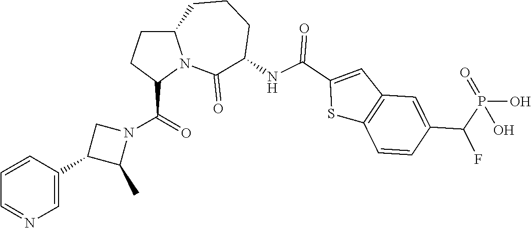 | | 2.18-2.07 (m, 2H), 2.05-1.94 (m, 3H), 1.92-1.85 (m, 2H), 1.84-1.74 (m, 2H) |
| 740 | ((R)-fluoro(2- (((3S,6S,8R,9aR)-8- methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 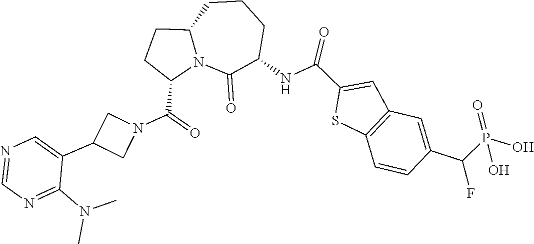 [SMILES: CN(C)c1ncncc1C1CN(C(=O)[C@@H]2CC[C@@H]3CCC[C@H](NC(=O)c4cc5cc(C(F)P(=O)(O)O)ccc5s4)C(=O)N32)C1] | 629.3 | ¹H NMR (400 MHz, DMSO-d₆) δ 8.82- 8.62 (m, 2H), 8.61- 8.49 (m, 1H), 8.23 (d, J = 19.6 Hz, 1H), 8.07- 7.92 (m, 2H), 7.69- 7.47 (m, 2H), 5.92- 5.74 (m, 1H), 4.88- 4.58 (m, 3H), 4.53- 4.42 (m, 2H), 4.38- 4.24 (m, 2H), 4.21- 4.16 (m, 1H), 4.07- 4.01 (m, 2H), 2.35 (d, J = 8.8 Hz, 3H), 2.27- 2.16 (m, 1H), 2.09- |
| | | | | 1.92 (m, 2H), 1.89- |
| | | | | 1.76 (m, 3H), 1.70 (d, |
| | | | | J = 12.8 Hz, 1H), 1.58- |
| | | | | 1.35 (m, 2H), 1.01- |
| | | | | 0.90 (m, 3H) |
| 741 | ((S)-(2- (((3S,6S,9aS)-3-(3- (4-(azetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 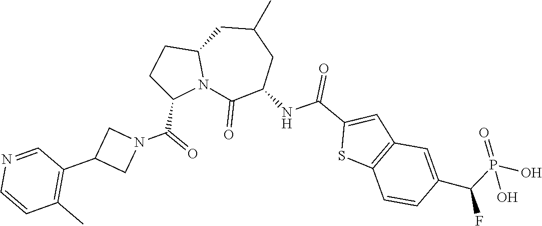 | 656.2 | ¹H NMR (400 MHz, CD₃OD) δ 8.22-8.08 (m, 1H), 8.06-8.00 (m, 1H), 7.98 (d, J = 12.4 Hz, 1H), 7.89 (d, J = 7.6 Hz, 1H), 7.87- 7.78 (m, 1H), 7.60 (t, J = 8.4 Hz, 1H), 6.45-6.31 (m, 1H), 5.86-5.63 (m, 1H), 4.73 (d, J = 4.0 Hz, 2H), 4.62-4.56 (m, 3H), 4.54-4.51 (m, 2H), 4.46-4.40 (m, |
| | | | | 3H), 4.32 (d, J = 8.4 |
| | | | | Hz, 1H), 4.24 (d, J = |
| | | | | 6.0 Hz, 1H), 4.12- |
| | | | | 4.03 (m, 2H), 2.52- |
| | | | | 2.43 (m, 1H), 2.36- |
| | | | | 2.25 (m, 1H), 2.18- |
| | | | | 2.06 (m, 2H), 2.05- |
| | | | | 1.89 (m, 4H), 1.85- |
| | | | | 1.79 (m, 2H) |
| 742 | ((R)-(2- (((3S,6S,9aS)-3-(3- (4-(azetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 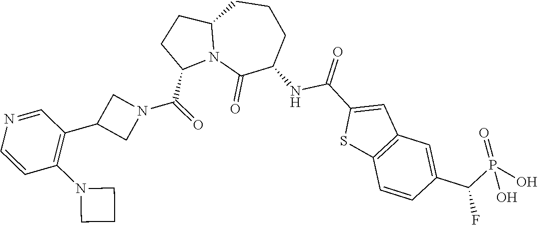 [SMILES: O=C(N[C@H]1CCC[C@H]2CC[C@@H](C(=O)N3CC(c4cnccc4N4CCC4)C3)N2C1=O)c1cc2cc([C@@H](F)P(=O)(O)O)ccc2s1] | 656.2 | ¹H NMR (400 MHz, DMSO-d₆) δ 8.78- 8.63 (m, 1H), 8.35- 8.20 (m, 1H), 8.19- 8.08 (m, 1H), 8.08- 7.97 (m, 1H), 7.96- 7.87 (m, 1H), 7.57- 7.40 (m, 1H), 6.53- 6.27 (m, 1H), 5.88- 5.61 (m, 1H), 4.73- 4.59 (m, 2H), 4.53 (t, J = 8.4 Hz, 1H), 4.46- 4.41 (m, 1H), 4.41- 4.24 (m, 5H), 4.23- |
| | | | | 4.08 (m, 2H), 4.07- |
| | | | | 3.93 (m, 3H), 2.41- |
| | | | | 2.28 (m, 2H), 2.25- |
| | | | | 2.14 (m, 1H), 2.08- |
| | | | | 1.92 (m, 2H), 1.90- |
| | | | | 1.77 (m, 3H), 1.75- |
| | | | | 1.66 (m, 2H) |
| 743 | ((2-(((3S,6S,9aS)-3- (3-(5-cyano-1- methyl-1H-pyrazol- 4-yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 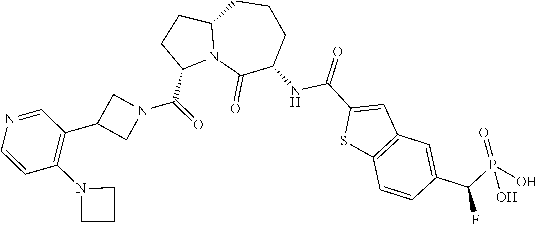 [SMILES: O=C(N[C@H]1CCC[C@H]2CC[C@@H](C(=O)N3CC(c4cnccc4N4CCC4)C3)N2C1=O)c1cc2cc([C@H](F)P(=O)(O)O)ccc2s1] | 629.3 | ¹H NMR (400 MHz, CDCl₃) δ 8.16-7.65 (m, 2H), 7.64-7.32 (m, 3H), 6.07-5.53 (m, 1H), 4.94-4.61 (m, 1H), 4.59-4.23 (m, 3H), 4.20-4.06 (m, 2H), 4.00 (s, 3H), 3.92 (d, J = 4.0 Hz, 2H), 2.36-2.18 (m, 1H), 2.13-1.91 (m, 4H), 1.90-1.51 (m, 5H |
| 744 | ((1R)-fluoro(2- (((3S,6S,8R,9aR)-3- (3-(4- isopropoxypyridin- 3-yl)azetidine-1- carbonyl)-8-methyl- 5-oxooctahydro-1H- pyrrolo[1,2- a]azepin-6- yl)carbamoyl)-2,3- dihydrobenzo[b] thiophen-5- yl)methyl) phosphonic acid | 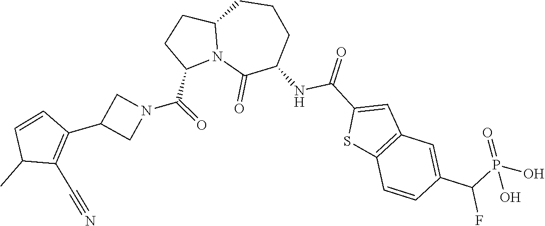 [SMILES: CC1C=CC(C2CN(C(=O)[C@@H]3CC[C@@H]4CCC[C@H](NC(=O)c5cc6cc(C(F)P(=O)(O)O)ccc6s5)C(=O)N43)C2)=C1C#N] | 673.3 | ¹H NMR (400 MHz, CD₃OD) δ 8.23-8.18 (m, 2H), 7.98-7.97 (m, 1H), 7.86-7.82 (m, 2H), 7.56-7.54 (m, 1H), 7.36-7.33 (m, 1H), 5.90-5.77 (m, 1H), 4.78-4.76 (m, 1H), 4.61-4.50 (m, 3H), 4.30-4.24 (m, 2H), 4.10-3.98 (m, 3H), 2.33-2.31 (m, 1H), 2.15-2.00 (m, 3H), 1.98-1.92 |
| | | | | (m, 2H), 1.82-1.75 |
| | | | | (m, 1H), 1.64-1.61 |
| | | | | (m, 2H), 1.51-1.42 |
| | | | | (m, 6H), 1.06-1.04 |
| | | | | (m, 3H) |
| 745 | ((S)-fluoro(2- (((3S,6S,9aS)-3-(3-(4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 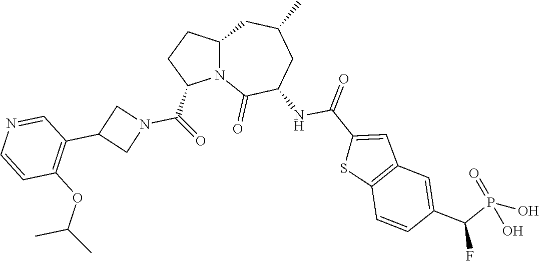 [SMILES: CC(C)Oc1ccncc1C1CN(C(=O)[C@@H]2CC[C@@H]3C[C@H](C)C[C@H](NC(=O)c4cc5cc([C@H](F)P(=O)(O)O)ccc5s4)C(=O)N32)C1] | 686.1 | ¹H NMR (400 MHz, CD₃OD) δ 8.36-8.16 (m, 1H), 8.16-8.00 (m, 1H), 7.98-7.89 (m, 1H), 7.88-7.81 (m, 1H), 7.80-7.72 (m, 1H), 7.64-7.41 (m, 1H), 7.26-6.85 (m, 1H), 5.89-5.58 (m, 1H), 4.81-4.76 (m, 1H), 4.75-4.68 (m, 1H), 4.61-4.45 (m, 2H), 4.43-4.26 (m, 1H), 4.17-4.01 (m, 3H), 3.88-3.66 (m, 4H), 3.31-3.11 |
| | | | | (m, 4H), 2.39-2.25 |
| | | | | (m, 1H), 2.20-2.06 |
| | | | | (m, 2H), 2.03-1.76 |
| | | | | (m, 7H) |
| 746 | ((R)-fluoro(2- (((3S,6S,9aS)-3-(3-(4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 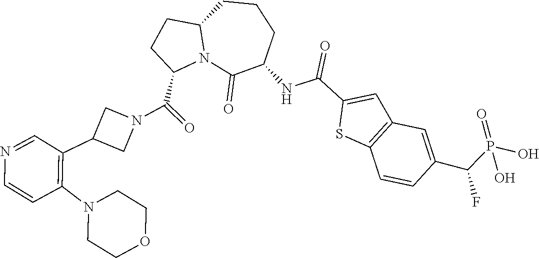 [SMILES: O=C(N[C@H]1CCC[C@H]2CC[C@@H](C(=O)N3CC(c4cnccc4N4CCOCC4)C3)N2C1=O)c1cc2cc([C@@H](F)P(=O)(O)O)ccc2s1] | 686.1 | ¹H NMR (400 MHz, CD₃OD) δ 8.37-8.12 (m, 1H), 8.12-7.98 (m, 1H), 7.97-7.84 (m, 1H), 7.84-7.75 (m, 1H), 7.72-7.52 (m, 1H), 7.58-7.47 (m, 1H), 7.22-6.85 (m, 1H), 5.91-5.55 (m, 1H), 4.84-4.78 (m, 1H), 4.73-4.65 (m, 1H), 4.59-4.44 (m, 2H), 4.42-4.25 (m, 1H), 4.16-3.98 (m, 3H), 3.76-3.75 (m, 4H), 3.30-3.13 |
| | | | | (m, 4H), 2.37-2.26 |
| | | | | (m, 1H), 2.22-2.07 |
| | | | | (m, 2H), 2.06-1.75 |
| | | | | (m, 7H) |
| 747 | ((R)-fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 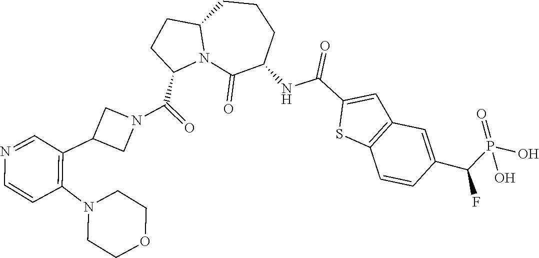 [SMILES: O=C(N[C@H]1CCC[C@H]2CC[C@@H](C(=O)N3CC(c4cnccc4N4CCOCC4)C3)N2C1=O)c1cc2cc([C@H](F)P(=O)(O)O)ccc2s1] | 631.3 | ¹H NMR (400 MHz, CD₃OD) δ 8.36-8.23 (m, 2H), 8.01-7.92 (m, 1H), 7.91-7.77 (m, 2H), 7.55 (d, J = 7.6 Hz, 1H), 7.41- 7.22 (m, 1H), 5.86- 5.65 (m, 1H), 4.84- 4.70 (m, 2H), 4.65- 4.55 (m, 1H), 4.53- 4.46 (m, 1H), 4.34- 4.24 (m, 1H), 4.18- 3.97 (m, 6H), 2.37- 2.25 (m, 1H), 2.17- 2.05 (m, 2H), 2.03- 1.80 (m, 7H) |
| 748 | ((R)-fluoro(2- (((3S,6S,8S,9aR)-3- (3-(4- isopropoxypyridin- 3-yl)azetidine-1- carbonyl)-8-methyl- 5-oxooctahydro-1H- pyrrolo[1,2- a]azepin-6- yl)carbamoyl)benzo [b]thiophen-5- yl)methyl) phosphonic acid | 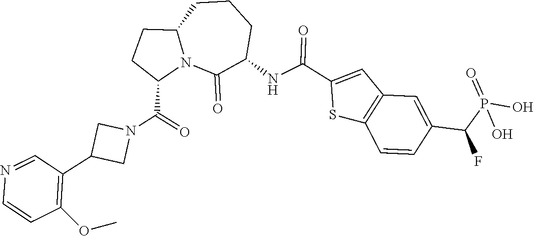 | 673.1 | ¹H NMR (400 MHz, DMSO-d₆) δ 8.79- 8.65 (m, 1H), 8.54- 8.41 (m, 2H), 8.29- 8.17 (m, 1H), 8.07- 7.93 (m, 2H), 7.57- 7.49 (m, 1H), 7.36- 7.22 (m, 1H), 5.92- 5.72 (m, 1H), 4.97- 4.82 (m, 2H), 4.73- 4.53 (m, 1H), 4.44- 4.29 (m, 2H), 4.23- 4.13 (m, 2H), 4.10- 3.92 (m, 3H), 2.27- 1.97 (m, 4H), 1.92- |
| | | | | 1.74 (m, 4H), 1.71- |
| | | | | 1.61 (m, 1H), 1.53- |
| | | | | 1.43 (m, 1H), 1.35- |
| | | | | 1.29 (m, 6H), 1.12 (t, |
| | | | | J = 6.4 Hz, 3H) |
| 749 | ((R)-fluoro(2- (((3S,6S,8S,9aR)-3- (3-(4- methoxypyridin-3- yl)azetidine-1- carbonyl)-8-methyl- 5-oxooctahydro-1H- pyrrolo[1,2- a]azepin-6- yl)carbamoyl)benzo [b]thiophen-5- yl)methyl) phosphonic acid | 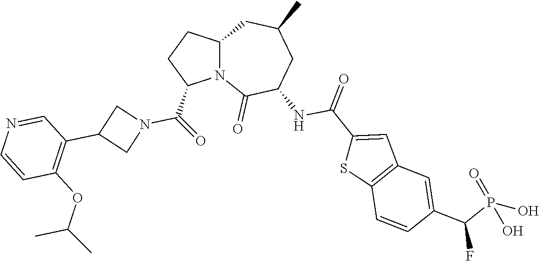 [SMILES: CC(C)Oc1ccncc1C1CN(C(=O)[C@@H]2CC[C@@H]3C[C@@H](C)C[C@H](NC(=O)c4cc5cc([C@H](F)P(=O)(O)O)ccc5s4)C(=O)N32)C1] | 645.1 | ¹H NMR (400 MHz, DMSO-d₆) δ 8.74- 8.72 (m, 1H), 8.43- 8.35 (m, 2H), 8.24- 8.18 (m, 1H), 8.03- 7.94 (m, 2H), 7.52 (t, J = 9.6 Hz, 1H), 7.22- 7.09 (m, 1H), 5.86- 5.72 (m, 1H), 4.87- 4.74 (m, 1H), 4.71- 4.53 (m, 1H), 4.43- 4.37 (m, 2H), 4.21- 4.15 (m, 2H), 4.02- 3.96 (m, 2H), 3.91- 3.89 (m, 3H), 2.19- 2.09 (m, 2H), 2.07- |
| | | | | 2.03 (m, 1H), 1.91- |
| | | | | 1.88 (m, 2H), 1.81- |
| | | | | 1.79 (m, 1H), 1.70- |
| | | | | 1.62 (m, 1H), 1.49- |
| | | | | 1.45 (m, 1H), 1.27- |
| | | | | 1.23 (m, 1H), 1.13- |
| | | | | 1.09 (m, 3H) |
| 750 | ((2-(((3S,6S,9aS)-3- (3-(4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 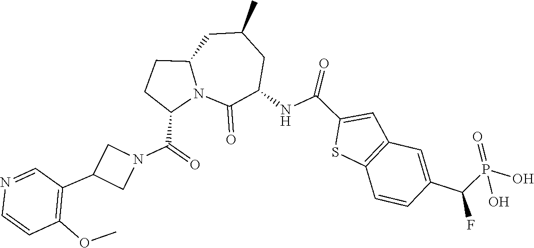 [SMILES: COc1ccncc1C1CN(C(=O)[C@@H]2CC[C@@H]3C[C@@H](C)C[C@H](NC(=O)c4cc5cc([C@H](F)P(=O)(O)O)ccc5s4)C(=O)N32)C1] | 668.2 | ¹H NMR (400 MHz, CD₃OD) δ 8.49-8.30 (m, 1H), 8.29-8.02 (m, 1H), 8.01-7.88 (m, 1H), 7.87-7.77 (m, 1H), 7.76-7.70 (m, 1H), 7.46-7.34 (m, 1H), 7.33-7.10 (m, 1H), 5.00-4.93 (m, 1H), 4.92-4.87 (m, 2H), 4.79-4.68 (m, 2H), 4.54 (t, J = 6.8 Hz, 2H), 4.47- 4.31 (m, 1H), 4.24- 4.14 (m, 1H), 4.14- |
| | | | | 4.02 (m, 2H), 3.82 (d, |
| | | | | J = 4.0 Hz, 4H), 3.38 |
| | | | | (d, J = 4.0 Hz, 1H), |
| | | | | 3.27-3.14 (m, 2H), |
| | | | | 2.39-2.26 (m, 1H), |
| | | | | 2.23-1.75 (m, 9H) |
| 751 | (fluoro(2- (((3S,6S,9aS)-3-(3- (4-fluoro-1-methyl- 1H-pyrazol-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 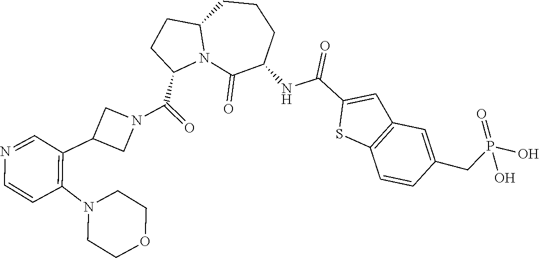 [SMILES: O=C(N[C@H]1CCC[C@H]2CC[C@@H](C(=O)N3CC(c4cnccc4N4CCOCC4)C3)N2C1=O)c1cc2cc(CP(=O)(O)O)ccc2s1] | 622.3 | ¹H NMR (400 MHz, CD₃OD) δ 8.06 (m, 2H), 7.94 (s, 1H), 7.63-7.55 (m, 1H), 7.35 (m, 1H), 5.91- 5.73 (m, 1H), 4.95- 4.92 (m, 1H), 4.77- 4.66 (m, 2H), 4.61- 4.53 (m, 1H), 4.46 (s, 1H), 4.31 (s, 1H), 4.19 (m, 1H), 4.12- 4.02 (m, 1H), 3.73 (d, J = 7.8 Hz, 3H), 2.35- 2.26 (m, 1H), 2.17- 2.11 (m, 1H), 2.10- 2.01 (m, 2H), 2.00- |
| | | | | 1.93 (m, 2H), 1.92- |
| | | | | 1.87 (m, 2H), 1.86- |
| | | | | 1.78 (m, 2H) |
| 752 | (fluoro(2- (((3S,6S,9aS)-3-(3- (5-fluoro-2- methoxyphenyl) azetidine-1- carbonyl)-5- oxooctahydro-1H- pyrrolo[1,2- a]azepin-6- yl)carbamoyl)-2,3- dihydrobenzo[b] thiophen-5- yl)methyl) phosphonic acid | 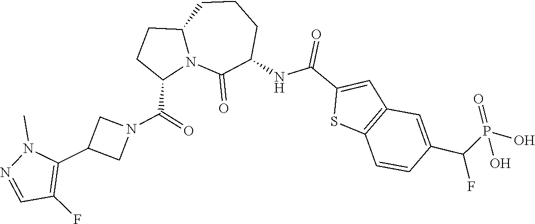 [SMILES: Cn1ncc(F)c1C1CN(C(=O)[C@@H]2CC[C@@H]3CCC[C@H](NC(=O)c4cc5cc(C(F)P(=O)(O)O)ccc5s4)C(=O)N32)C1] | 648.0 | ¹H NMR (400 MHz, CD₃OD) δ 8.13-8.01 (m, 2H), 7.97-7.94 (m, 1H), 7.60-7.57 (m, 1H), 7.21-7.05 (m, 1H), 7.01-6.90 (m, 2H), 5.93-5.76 (m, 1H), 4.77-4.74 (m, 2H), 4.64-4.53 (m, 2H), 4.40-4.39 (m, 1H), 4.18-4.02 (m, 3H), 3.82 (s, 3H), 2.36-2.26 (m, 1H), 2.18-2.05 (m, 2H), |
| | | | | 2.03-2.01(m, 1H), |
| | | | | 1.99-1.78 (m, 6H) |
| 753 | ((S)-fluoro(2- (((3S,6S,8R,9aR)-8- methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 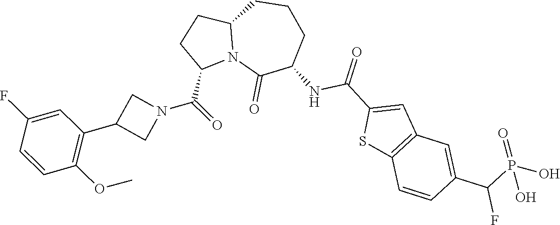 [SMILES: COc1ccc(F)cc1C1CN(C(=O)[C@@H]2CC[C@@H]3CCC[C@H](NC(=O)c4cc5cc(C(F)P(=O)(O)O)ccc5s4)C(=O)N32)C1] | 629.4 | ¹H NMR (400 MHz, DMSO-d₆) δ 8.88- 8.60 (m, 2H), 8.57- 8.46 (m, 1H), 8.23 (d, J = 19.6 Hz, 1H), 8.07- 7.91 (m, 2H), 7.62- 7.48 (m, 2H), 5.94- 5.69 (m, 1H), 4.87- 4.75 (m, 1H), 4.71- 4.59 (m, 2H), 4.52- 4.42 (m, 2H), 4.39- 4.24 (m, 2H), 4.17 (d, J = 7.6 Hz, 1H), 4.09- 4.00 (m, 2H), 2.33 |
| | | | | (d, J = 8.0 Hz, 3H), |
| | | | | 2.26-2.15 (m, 1H), |
| | | | | 2.08-1.92 (m, 2H), |
| | | | | 1.89-1.75 (m, 3H), |
| | | | | 1.70 (d, J = 11.2 Hz, |
| | | | | 1H), 1.56-1.35 (m, |
| | | | | 2H), 1.03-0.88 (m, |
| | | | | 3H) |
| 754 | (fluoro(2- (((3S,6S,9aS)-3-(3- (4-fluoro-1-methyl- 1H-pyrazol-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 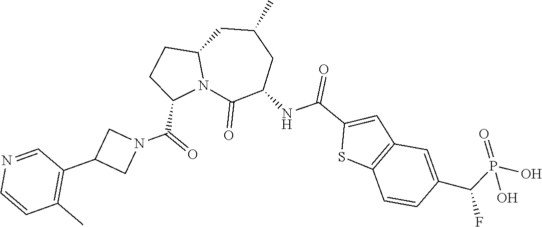 | 622.1 | ¹H NMR (400 MHz, CD₃OD) δ 8.07 (d, J = 8.0 Hz, 1H), 8.04- 7.98 (m, 1H), 7.98- 7.90 (m, 1H), 7.55 (m, 2H), 5.91-5.76 (m, 1H), 4.78-4.71 (m, 1H), 4.65-4.55 (m, 2H), 4.43-4.35 (m, 1H), 4.34-4.23 (m, 1H), 4.12-4.02 (m, 2H), 3.97 (m, 1H), 3.82-3.74 (m, 3H), 2.36-2.22 (m, |
| | | | | 1H), 2.12 (m, 1H), |
| | | | | 2.09-2.03 (m, 1H), |
| | | | | 2.02-1.93 (m, 3H), |
| | | | | 1.92-1.87 (m, 2H), |
| | | | | 1.86-1.78 (m, 2H) |
| 755 | ((S)-fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 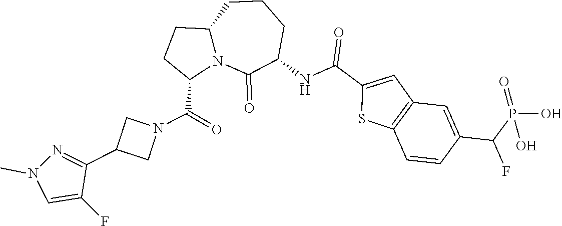 [SMILES: Cn1cc(F)c(C2CN(C(=O)[C@@H]3CC[C@@H]4CCC[C@H](NC(=O)c5cc6cc(C(F)P(=O)(O)O)ccc6s5)C(=O)N43)C2)n1] | 615.2 | ¹H NMR (400 MHz, CD₃OD) δ 8.64 (d, J = 5.6 Hz, 1H), 8.42- 8.27 (m, 1H), 8.03- 7.49 (m, 5H), 5.89- 5.68 (m, 1H), 4.74 (d, J = 10.4 Hz, 2H), 4.66- 4.50 (m, 2H), 4.46- 4.22 (m, 2H), 4.16- 4.02 (m, 2H), 2.53- 2.40 (m, 3H), 2.36- 2.27 (m, 1H), 2.18- 1.81 (m, 9H) |
| 756 | ((S)-fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 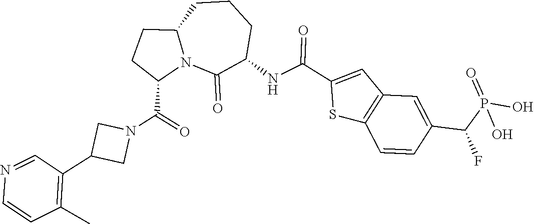 | 631.1 | ¹H NMR (400 MHz, CD₃OD) δ 8.36-8.28 (m, 2H), 8.06-7.98 (m, 2H), 7.85 (t, J = 9.6 Hz, 1H), 7.63 (t, J = 8.0 Hz, 1H), 7.13- 7.05 (m, 1H), 5.73- 5.57 (m, 1H), 4.77- 4.65 (m, 2H), 4.64- 4.59 (m, 1H), 4.59- 4.53 (m, 1H), 4.44- 4.35 (m, 1H), 4.22- 4.11 (m, 1H), 4.10- 4.01 (m, 2H), 3.97 (d, |
| | | | | J = 6.0 Hz, 3H), 2.37- |
| | | | | 2.25 (m, 1H), 2.19- |
| | | | | 2.05 (m, 2H), 2.04- |
| | | | | 1.99 (m, 1H), 1.99- |
| | | | | 1.90 (m, 3H), 1.90- |
| | | | | 1.78 (m, 3H) |
| 757 | (fluoro(2- (((3S,6S,9aS)-3-(3- (4-fluorothiophen-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 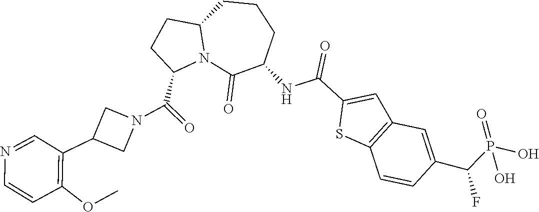 [SMILES: COc1ccncc1C1CN(C(=O)[C@@H]2CC[C@@H]3CCC[C@H](NC(=O)c4cc5cc([C@@H](F)P(=O)(O)O)ccc5s4)C(=O)N32)C1] | 624.1 | ¹H NMR (400 MHz, CD₃OD) δ 8.09-8.08 (m, 1H), 8.04 (s, 1H), 7.97-7.94(m, 1H), 7.60-7.58 (m, 1H), 7.13-6.62 (m, 2H), 5.93-5.73 (m, 1H), 4.76-4.74 (m, 1H), 4.62-4.48 (m, 2H), 4.45-4.17 (m, 2H), 4.10-3.82 (m, 3H), 2.37-2.24 (m, 1H), 2.16-2.06 (m, 2H), |
| | | | | 2.05-1.99 (m, 1H), |
| | | | | 1.99-1.88 (m, 4H), |
| | | | | 1.87-1.81 (m, 2H) |
| 758 | ((2- (((1R,3S,6S,10aR,Z)- 3-((rel-trans)-3- cyano-4- phenylpyrrolidine- 1-carbonyl)-1- hydroxy-5-oxo- 1,2,3,5,6,7,10,10a- octahydropyrrolo[1, 2-a]azocin-6- yl)carbamoyl)benzo [b]thiophen-5- yl)difluoromethyl) phosphonic acid | 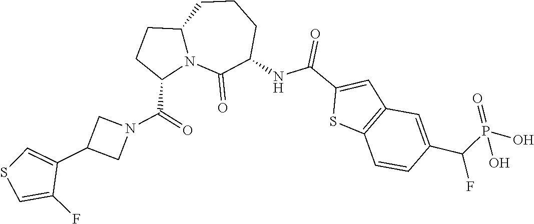 [SMILES: O=C(N[C@H]1CCC[C@H]2CC[C@@H](C(=O)N3CC(c4cscc4F)C3)N2C1=O)c1cc2cc(C(F)P(=O)(O)O)ccc2s1] | 685.1 | ¹H NMR (400 MHz, CD₃OD) δ 8.19 (d, J = 10.0 Hz, 1H), 8.11- 8.06 (m, 1H), 7.95 (m, 1H), 7.78-7.70 (m, 1H), 7.48-7.31 (m, 5H), 5.89-5.68 (m, 2H), 5.12-4.96 (m, 2H), 4.76-4.63 (m, 2H), 4.49-4.31 (m, 1H), 4.24-3.94 (m, 2H), 3.89-3.76 (m, 1H), 3.71-3.51 (m, 2H), 3.08-2.93 (m, 1H), 2.68-2.58 |
| | | | | (m, 1H), 2.54-2.35 |
| | | | | (m, 2H), 2.29-2.16 |
| | | | | (m, 1H), 2.11-2.01 |
| | | | | (m, 1H) |
| 759 | (difluoro(2- (((3S,6S,9aR)-8- methyl-5-oxo-3-(3- (pyridin-3- yl)azetidine-1- carbonyl)octahydro- 1H-pyrrolo[1,2- a]azepin-6- yl)carbamoyl)benzo [b]thiophen-5- yl)methyl) phosphonic acid | 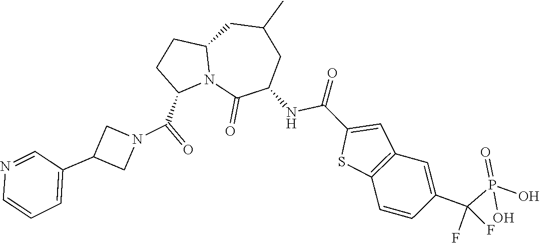 | 633.1 | ¹H NMR (400 MHz, CD₃OD) δ 8.61-8.54 (m, 2H), 8.50-8.44 (m, 1H), 8.37-8.31 (m, 1H), 8.19 (d, J = 3.8 Hz, 1H), 8.13- 8.07 (m, 2H), 7.94 (d, J = 8.5 Hz, 1H), 7.74 (d, J = 8.6 Hz, 1H), 7.61-7.54 (m, 1H), 4.78-4.66 (m, 3H), 4.65-4.38 (m, 4H), 4.33-4.24 (m, 1H), 4.09-4.00 (m, 3H), 2.38-2.25 (m, 2H), |
| | | | | 2.00-1.90 (m, 3H), |
| | | | | 1.80 (d, J = 14.4 Hz, |
| | | | | 1H), 1.68-1.57 (m, |
| | | | | 2H), 1.25-1.21 (m, |
| | | | | 1H), 1.10-1.00 (m, |
| | | | | 3H) |
| 760 | ((2-(((3S,6S,9aS)-3- (4-(1H-pyrazol-3- yl)piperidine-1- carbonyl)-5- oxooctahydro-1H- pyrrolo[1,2- a]azepin-6- yl)carbamoyl)benzo [b]thiophen-5- yl)fluoromethyl) phosphonic acid | 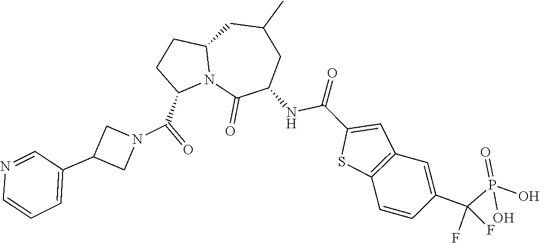 | 618.1 | ¹H NMR (400 MHz, DMSO-d₆) δ 8.70 (m, 1H), 8.23 (m, 1H), 8.03 (d, J = 8.4 Hz, 1H), 7.97 (m, 1H), 7.53 (d, J = 8.6 Hz, 1H), 7.49 (d, J = 4.6 Hz, 1H), 6.07 (d, J = 17.4 Hz, 1H), 5.77 (d, J = 8.0 Hz, 1H), 5.00-4.91 (m, 1H), 4.66-4.60 (m, 1H), 4.36 (d, J = 11.8 Hz, 1H), 3.62 (m, 1H), 3.23-3.12 (m, 1H), |
| | | | | 2.95-2.83 (m, 1H), |
| | | | | 2.71 (t, J = 12.0 Hz, |
| | | | | 1H), 2.27-2.16 (m, |
| | | | | 1H), 2.13-2.02 (m, |
| | | | | 1H), 2.01-1.82 (m, |
| | | | | 4H), 1.81-1.66 (m, |
| | | | | 4H), 1.65-1.36 (m, |
| | | | | 2H), 1.28-1.22 (m, |
| | | | | 2H) |
| 761 | ((2-(((3S,6S,10aS)- 3-(3-((2,2- difluoroethyl)amino)- 3-(pyridin-2- yl)azetidine-1- carbonyl)-5- oxodecahydropyrrol o[1,2-a]azocin-6- yl)carbamoyl)benzo [b]thiophen-5- yl)fluoromethyl) phosphonic acid | 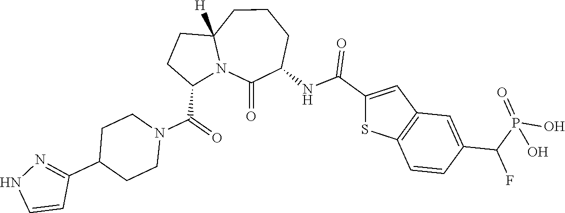 [SMILES: O=C(N[C@H]1CCC[C@H]2CC[C@@H](C(=O)N3CCC(c4cc[nH]n4)CC3)N2C1=O)c1cc2cc(C(F)P(=O)(O)O)ccc2s1] | 694.4 | ¹H NMR (400 MHz, DMSO-d₆) δ 8.85- 8.74 (m, 1H), 8.57 (dd, J = 4.0, 15.6 Hz, 1H), 8.29-8.18 (m, 1H), 8.02-7.80 (m, 3H), 7.64-7.53 (m, 1H), 7.52-7.43 (m, 1H), 7.35-7.30 (m, 1H), 6.08-5.64 (m, 2H), 4.98-4.88 (m, 1H), 4.71 (d, J = 8.8 Hz, 1H), 4.53 (d, J = 8.4 Hz, 1H), 4.42 (d, |
| | | | | J = 8.8 Hz, 1H), 4.39- |
| | | | | 4.31 (m, 1H), 4.30- |
| | | | | 4.17 (m, 3H), 4.06- |
| | | | | 3.90 (m, 1H), 3.89- |
| | | | | 3.80 (m, 1H), 2.22- |
| | | | | 2.02 (m, 2H), 1.91- |
| | | | | 1.72 (m, 7H), 1.65- |
| | | | | 1.48 (m, 3H) |
| 762 | (difluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 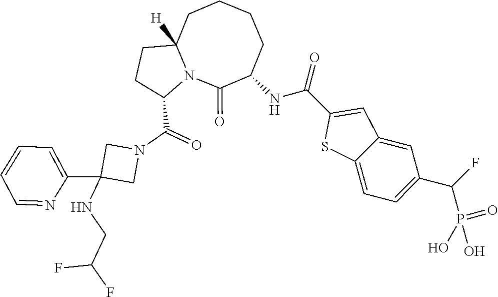 [SMILES: O=C(N[C@H]1CCCC[C@H]2CC[C@@H](C(=O)N3CC(NCC(F)F)(c4ccccn4)C3)N2C1=O)c1cc2cc(C(F)P(=O)(O)O)ccc2s1] | 637.1 | ¹H NMR (400 MHz, DMSO-d₆) δ 8.79 (t, J = 7.2 Hz, 1H), 8.68 (dd, J = 3.6, 10.4 Hz, 1H), 8.57-8.48 (m, 1H), 8.25 (d, J = 9.6 Hz, 1H), 8.05-8.01 (m, 1H), 7.97 (d, J = 8.4 Hz, 1H), 7.65- 7.60 (m, 1H), 7.33 (dt, J = 5.6, 11.2 Hz, 1H), 4.70-4.57 (m, 2H), 4.55-4.40 (m, 2H), 4.37-4.30 (m, 1H), 4.29-4.22 (m, 1H), 4.16-4.04 (m, 2H), 4.02-3.94 (m, 2H), 2.22-2.15 (m, |
| | | | | 1H), 2.08-2.00 (m, |
| | | | | 1H), 2.00-1.91 (m, |
| | | | | 2H), 1.83-1.79 (m, |
| | | | | 2H), 1.75-1.66 (m, |
| | | | | 4H) |
| 763 | (fluoro(2- (((3S,6S,9aS)-3-(4- methyl-1,2,3,4- tetrahydro- quinoxaline-1- carbonyl)-5- oxooctahydro-1H- pyrrolo[1,2- a]azepin-6- yl)carbamoyl)benzo [b]thiophen-5- yl)methyl) phosphonic acid | 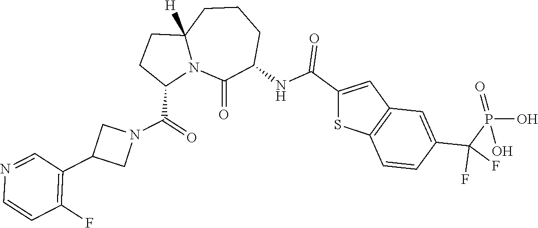 [SMILES: O=C(N[C@H]1CCC[C@H]2CC[C@@H](C(=O)N3CC(c4cnccc4F)C3)N2C1=O)c1cc2cc(C(F)(F)P(=O)(O)O)ccc2s1] | 615.1 | ¹H NMR (400 MHz, CD₃OD) δ 8.09-8.00 (m, 2H), 7.94 (d, J = 8.4 Hz, 1H), 7.58 (d, J = 8.6 Hz, 1H), 7.34- 7.24 (m, 1H), 7.14- 7.04 (m, 1H), 6.76 (d, J = 8.4 Hz, 1H), 6.63 (t, J = 7.4 Hz, 1H), 5.91-5.72 (m, 1H), 5.27-5.16 (m, 1H), 4.75-4.67 (m, 1H), 4.05 (d, J = 1.8, 4.0 Hz, 1H), 3.92-3.72 |
| | | | | (m, 2H), 3.48-3.37 |
| | | | | (m, 2H), 2.97 (s, 3H), |
| | | | | 2.31-2.18 (m, 1H), |
| | | | | 2.08-1.79 (m, 9H) |
| 764 | (fluoro(2- (((3S,6S,9aS)-3-(3-(4- methoxypyrimidin- 5-yl)azetidine-1- carbonyl)-5- oxooctahydro-1H- pyrrolo[1,2- a]azepin-6- yl)carbamoyl)benzo [b]thiophen-5- yl)methyl) phosphonic acid | 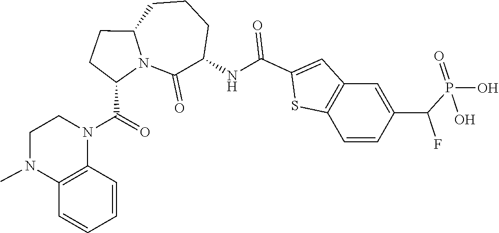 [SMILES: CN1CCN(C(=O)[C@@H]2CC[C@@H]3CCC[C@H](NC(=O)c4cc5cc(C(F)P(=O)(O)O)ccc5s4)C(=O)N32)c2ccccc21] | 632.2 | ¹H NMR (400 MHz, DMSO-d₆) δ 8.82- 8.73 (m, 1H), 8.72- 8.52 (m, 1H), 8.28- 8.16 (m, 1H), 8.09- 8.01 (m, 1H), 8.01- 7.93 (m, 1H), 7.61- 7.48 (m, 1H), 5.91- 5.74 (m, 1H), 4.72- 4.61 (m, 2H), 4.55- 4.49 (m, 1H), 4.46- 4.43 (m, 1H), 4.35- 4.29 (m, 1H), 4.20- 4.14 (m, 2H), 4.05- 4.01 (m, 1H), 3.95 (s, |
| | | | | 3H), 2.27-1.92 (m, |
| | | | | 4H), 1.89-1.68 (m, |
| | | | | 6H |
| 765 | ((2-(((3S,6S,9aS)-3- (3-(4-(azetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 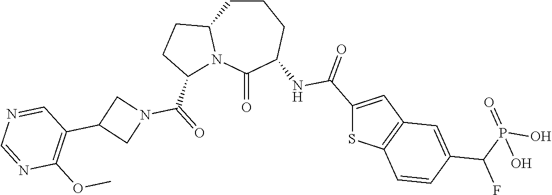 [SMILES: COc1ncncc1C1CN(C(=O)[C@@H]2CC[C@@H]3CCC[C@H](NC(=O)c4cc5cc(C(F)P(=O)(O)O)ccc5s4)C(=O)N32)C1] | 638.3 | ¹H NMR (400 MHz, DMSO-d₆) δ 8.79- 8.58 (m, 1H), 8.37- 8.09 (m, 2H), 8.08- 7.87 (m, 1H), 7.86- 7.74 (m, 1H), 7.45- 7.28 (m, 1H), 6.56- 6.31 (m, 1H), 4.73- 4.58 (m, 2H), 4.57- 4.49 (m, 1H), 4.47- 4.27 (m, 6H), 4.26- 4.10 (m, 2H), 4.07- |
| | | | | 3.96 (m, 3H), 3.12- |
| | | | | 3.02 (m, 2H), 2.43- |
| | | | | 2.29 (m, 2H), 2.26- |
| | | | | 2.15 (m, 1H), 2.06- |
| | | | | 1.91 (m, 2H), 1.89- |
| | | | | 1.77 (m, 3H), 1.75- |
| | | | | 1.61 (m, 2H) |
| 766 | ((R)-fluoro(2- (((3S,6S,8R,9aR)-3- (3-(4- methoxypyridin-3- yl)azetidine-1- carbonyl)-8-methyl- 5-oxooctahydro-1H- pyrrolo[1,2- a]azepin-6- yl)carbamoyl)benzo [b]thiophen-5- yl)methyl) phosphonic acid | 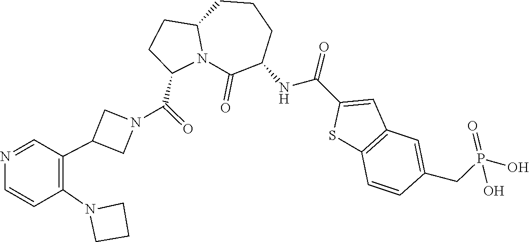 [SMILES: O=C(N[C@H]1CCC[C@H]2CC[C@@H](C(=O)N3CC(c4cnccc4N4CCC4)C3)N2C1=O)c1cc2cc(CP(=O)(O)O)ccc2s1] | 645.1 | ¹H NMR (400 MHz, DMSO-d₆) δ 8.80- 8.50 (m, 3H), 8.28- 8.18 (m, 1H), 8.06- 7.92 (m, 2H), 7.58- 7.36 (m, 2H), 5.92- 5.72 (m, 1H), 4.76- 4.61 (m, 2H), 4.55 (t, J = 8.4 Hz, 1H), 4.49- 4.40 (m, 2H), 4.36- 4.29 (m, 1H), 4.26- 4.14 (m, 2H), 4.02 (s, 5H), 2.27-2.15 (m, 1H), 2.08-1.93 (m, 2H), 1.81 (t, J = 9.2 |
| | | | | Hz, 3H), 1.70 (d, J = |
| | | | | 12.8 Hz, 1H), 1.56- |
| | | | | 1.36 (m, 2H), 0.96 (d, |
| | | | | J = 6.0 Hz, 3H) |
| 767 | ((2-(((3S,6S,9aS)-3- (3-(4-(3- methoxyazetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 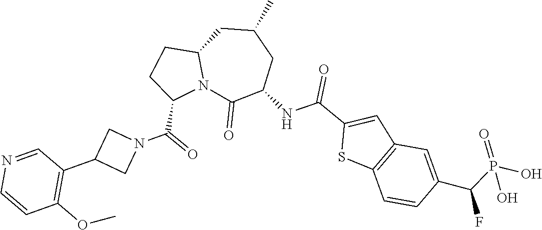 | 688.1 | ¹H NMR (400 MHz, CD₃OD) δ 8.08-7.33 (m, 6H), 6.28-6.07 (m, 1H), 4.80-4.67 (m, 2H), 4.67-4.17 (m, 6H), 4.16-3.95 (m, 5H), 3.36 (s, 1.5H), 3.28 (s, 1.5H), 3.18-2.96 (m, 2H), 2.36-2.22 (m, 1H), 2.18-1.75 (m, 9H) |
| 768 | (difluoro(2- (((3′S,6′S,10a′S)-3′- (3-methoxy-3-(1- methyl-1H- imidazol-2- yl)azetidine-1- carbonyl)-5′- oxooctahydro-5′H- spiro[cyclopropane- 1,8′-pyrrolo[1,2- a]azocin]-6′- yl)carbamoyl)benzo [b]thiophen-5- yl)methyl) phosphonic acid | 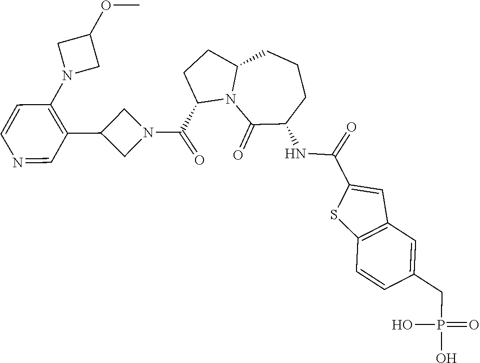 [SMILES: COC1CN(c2ccncc2C2CN(C(=O)[C@@H]3CC[C@@H]4CCC[C@H](NC(=O)c5cc6cc(CP(=O)(O)O)ccc6s5)C(=O)N43)C2)C1] | 692.1 | ¹H NMR (400 MHz, DMSO-d₆) δ 8.78- 8.66 (m, 1H), 8.29- 8.17 (m, 1H), 8.05- 7.93 (m, 2H), 7.65- 7.58 (m, 1H), 7.22 (dd, J = 9.8, 1.0 Hz, 1H), 6.86 (s, 1H), 4.97-4.84 (m, 1.5H), 4.78-4.68 (m, 1H), 4.51-4.26 (m, 3.5H), 4.11-3.98 (m, 1H), 3.56 (d, J = 5.9 Hz, 3H), 2.91 (d, J = 7.6 Hz, 3H), 2.25-2.15 (m, 0.5H), 2.14-1.99 (m, 1.5H), 1.98-1.65 (m, 5H), 1.62-1.43 (m, 3H), 0.53-0.29 (m, 4H) |
| 769 | (fluoro(2- (((3S,6S,9aS)-3-(3- (2-methylpyridin-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 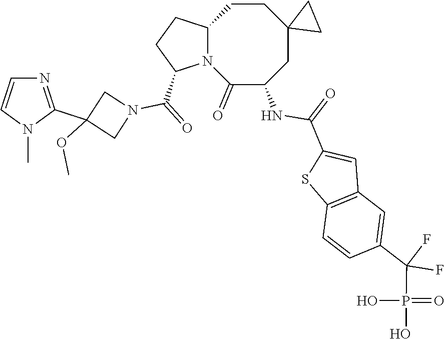 [SMILES: COC1(c2nccn2C)CN(C(=O)[C@@H]2CC[C@@H]3CCC4(CC4)C[C@H](NC(=O)c4cc5cc(C(F)(F)P(=O)(O)O)ccc5s4)C(=O)N32)C1] | 615.1 | ¹H NMR (400 MHz, CD₃OD) δ 8.21-7.95 (m, 3H), 7.85-7.24 (m, 4H), 5.86-5.60 (m, 1H), 5.00-4.92 (m, 1H), 4.78-4.66 (m, 2H), 4.56-4.34 (m, 2H), 4.25-3.92 (m, 3H), 2.60-2.46 (m, 3H), 2.38-2.25 (m, 1H), 2.23-1.75 (m, 9H) |
| 770 | ((2-(((3S,6S,9aS)-3- (8-acetyl-5-oxa-2,8- diazaspiro[3.5]nona ne-2-carbonyl)-5- oxooctahydro-1H- pyrrolo[1,2- a]azepin-6- yl)carbamoyl)benzo [b]thiophen-5- yl)fluoromethyl) phosphonic acid | 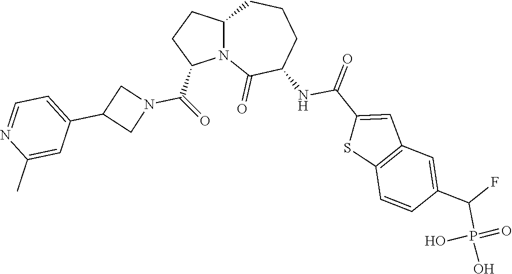 [SMILES: Cc1cc(C2CN(C(=O)[C@@H]3CC[C@@H]4CCC[C@H](NC(=O)c5cc6cc(C(F)P(=O)(O)O)ccc6s5)C(=O)N43)C2)ccn1] | 637.1 | ¹H NMR (400 MHz, CD₃OD) δ 8.13-8.00 (m, 2H), 7.97-7.91 (m, 1H), 7.64-7.56 (m, 1H), 5.89-5.68 (m, 1H), 4.77-4.71 (m, 1H), 4.61-4.31 (m, 2H), 4.19-4.01 (m, 2H), 4.00-3.76 (m, 3H), 3.75-3.65 (m, 2H), 3.65-3.47 (m, 2H), 2.36-2.24 (m, 1H), 2.18-2.02 (m, 5H), 2.02-1.90 (m, 3H), 1.89-1.75 (m, 3H), 1.39-1.35 (m, 2H) |
| 771 | ((S)-(2- (((3S,6S,9aS)-3-(3- (4-ethoxypyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 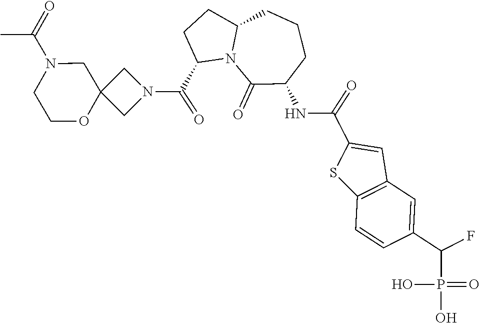 [SMILES: CC(=O)N1CCOC2(C1)CN(C(=O)[C@@H]1CC[C@@H]3CCC[C@H](NC(=O)c4cc5cc(C(F)P(=O)(O)O)ccc5s4)C(=O)N31)C2] | 645.1 | ¹H NMR (400 MHz, CD₃OD) δ 8.18-8.08 (m, 1H), 8.06-7.99 (m, 1H), 7.96-7.91 (m, 0.5H), 7.90-7.83 (m, 1H), 7.83-7.78 (m,1H), 7.78-7.73 (m, 0.5H), 7.71-7.65 (m, 0.5H), 7.58-7.52 (m, 0.5H), 7.14-7.02 (m, 1H), 5.79-5.59 (m, 1H), 4.63-4.44 (m, 3H), 4.38-4.02 (m, 6H), 4.02-3.83 (m, 1H), 2.39-2.23 (m, 1H), 2.20-1.72 (m, 9H), 1.52-1.40 (m, 3H). |
| 772 | ((R)-(2- (((3S,6S,9aS)-3-(3- (4-ethoxypyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 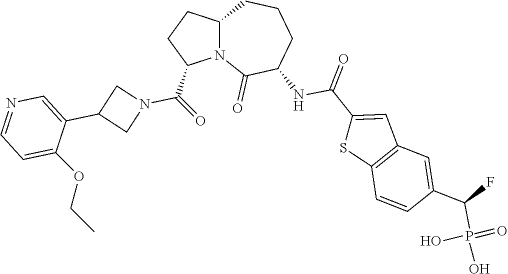 [SMILES: CCOc1ccncc1C1CN(C(=O)[C@@H]2CC[C@@H]3CCC[C@H](NC(=O)c4cc5cc([C@@H](F)P(=O)(O)O)ccc5s4)C(=O)N32)C1] | 645.1 | ¹H NMR (400 MHz, CD₃OD) δ 8.26-8.13 (m, 2H), 8.05-7.97 (m, 1H), 7.93-7.89 (m, 0.5H), 7.89-7.76 (m, 1.5H), 7.63-7.54 (m, 1H), 7.14-6.99 (m, 1H), 5.76-5.56 (m, 1H), 4.65-4.59 (m, 1H), 4.56-4.49 (m, 1H), 4.40-4.12 (m, 6H), 4.11-3.96 (m, 2H), 2.39-2.22 (m, 1H), 2.20-1.75 (m, 9H), 1.50-1.40 (m, 3H) |
| 773 | (difluoro(2- (((3S,6S,11aS)-3-(3- (morpholine-4- carbonyl)azetidine- 1-carbonyl)-5- oxodecahydro-1H- pyrrolo[1,2- a]azonin-6- yl)carbamoyl)benzo [b]thiophen-5- yl)methyl) phosphonic acid | 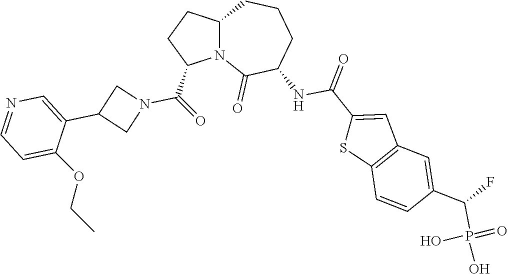 [SMILES: CCOc1ccncc1C1CN(C(=O)[C@@H]2CC[C@@H]3CCC[C@H](NC(=O)c4cc5cc([C@H](F)P(=O)(O)O)ccc5s4)C(=O)N32)C1] | 682.68 | ¹H NMR (400 MHz, DMSO-d₆) δ 8.90 (dd, J = 17.9, 6.4 Hz, 1H), 8.32 (s, 1H), 8.11 (d, J = 8.2 Hz, 1H), 8.04 (s, 1H), 7.57 (d, J = 8.5 Hz, 1H), 5.05-4.94 (m, 1H), 4.53 (dt, J = 35.3, 8.1 Hz, 2H), 4.30 (q, J = 6.9, 5.8 Hz, 2H), 4.21 (s, 2H), 4.08 (t, J = 9.5 Hz, 1H), 3.45 (s, 4H), 3.26 (t, J = 5.0 Hz, 3H), 3.06 (dd, J = 7.6, 5.1 Hz, 2H), 2.15- |
| | | | | 2.02 (m, 2H), 2.02- |
| | | | | 1.92 (m, 2H), 1.75 (q, |
| | | | | J = 14.3, 12.6 Hz, |
| | | | | 5H), 1.69-1.58 (m, |
| | | | | 2H), 1.49 (s, 2H), |
| | | | | 1.40 (d, J = 14.9 Hz, |
| | | | | 1H) |
| 774 | (difluoro(2- (((3S,6S,10aS)-5- oxo-3-(7′-oxo-6′,7′- dihydrospiro[azetidi ne-3,5′-pyrrolo[1,2- a]imidazole]-1- carbonyl)decahydro pyrrolo[1,2- a]azocin-6- yl)carbamoyl)benzo [b]thiophen-5- yl)methyl) phosphonic acid | 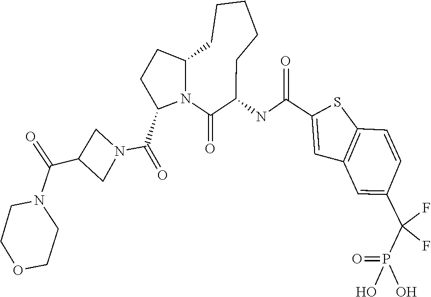 | 661.62 | ¹H NMR (600 MHz, DMSO-d₆) δ 8.98- 8.87 (m, 1H), 8.33 8.27 (m, 1H), 8.16- 7.95 (m, 3H), 7.56 (dd, J = 9.0, 5.2 Hz, 2H), 4.97-4.89 (m, 1H), 4.82 (dd, J = 40.6, 9.9 Hz, 1H), 4.58 (dd, J = 51.4, 10.0 Hz, 1H), 4.40- 4.17 (m, 4H), 3.03 (d, J = 7.4 Hz, 1H), 2.26- 2.01 (m, 3H), 1.85 (q, J = 12.4 Hz, 5H), 1.78-1.64 (m, 3H), 1.61 (s, 1H), 1.52 (d, J = 10.0 Hz, 1H) |
| 775 | (difluoro(2- (((3S,6S,9aS)-3-(3- (2-methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 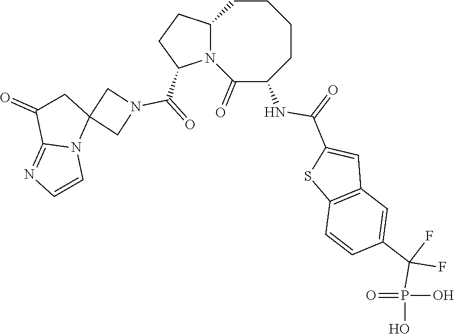 | 632.62 | ¹H NMR (500 MHz, DMSO-d₆) δ 8.74 (dd, J = 15.0, 7.7 Hz, 1H), 8.29 (s, 1H), 8.24 (d, J = 15.1 Hz, 1H), 8.07 (dd, J = 8.7, 3.6 Hz, 1H), 8.03 (s, 1H), 7.98-7.84 (m, 1H), 7.61-7.50 (m, 1H), 7.30 (d, J = 30.7 Hz, 1H), 4.81-4.50 (m, 3H), 4.47-4.38 (m, 2H), 4.28-4.21 (m, 1H), 4.09-4.04 (m, 1H), 2.38 (d, J = 6.4 Hz, 3H), 2.18 (d, J = 9.6 Hz, 1H), 1.98 (dd, J = 39.2, 10.0 Hz, 2H), 1.89-1.63 (m, 8H) |
| 776 | ((2-(((3S,6S,9aS)-3- (3-cyano-3-(pyridin- 2-yl)azetidine-1- carbonyl)-5- oxooctahydro-1H- pyrrolo[1,2- a]azepin-6- yl)carbamoyl)benzo [b]thiophen-5- yl)difluoromethyl) phosphonic acid | 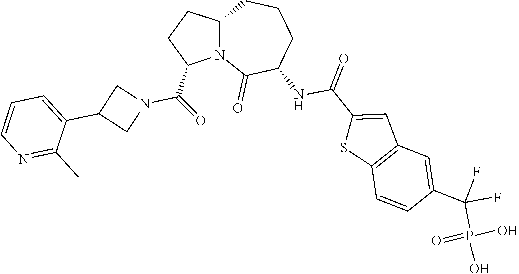 [SMILES: Cc1ncccc1C1CN(C(=O)[C@@H]2CC[C@@H]3CCC[C@H](NC(=O)c4cc5cc(C(F)(F)P(=O)(O)O)ccc5s4)C(=O)N32)C1] | 643.6 | ¹H NMR (600 MHz, DMSO-d₆) δ 8.83 (dd, J = 64.2, 7.7 Hz, 1H), 8.65 (dd, J = 31.9, 4.8 Hz, 1H), 8.32-8.24 (m, 1H), 8.11 (dd, J = 8.5, 5.1 Hz, 1H), 8.06 (d, J = 15.1 Hz, 1H), 7.98- 7.86 (m, 1H), 7.71 (t, J = 7.3 Hz, 1H), 7.57 (d, J = 8.7 Hz, 1H), 7.44 (ddd, J = 30.3, 7.6, 4.8 Hz, 1H), 5.04- 4.73 (m, 2H), 4.66 (dt, J = 21.8, 9.8 Hz, 1H), 4.48 (dd, J = |
| | | | | 16.5, 10.0 Hz, 1H), |
| | | | | 4.45-4.35 (m, 2H), |
| | | | | 4.34 (s, 1H), 4.00 (s, |
| | | | | 1H), 2.19 (dt, J = |
| | | | | 13.6, 7.2 Hz, 1H), |
| | | | | 2.09-1.99 (m, 1H), |
| | | | | 1.94 (s, 1H), 1.87- |
| | | | | 1.75 (m, 4H), 1.75- |
| | | | | 1.50 (m, 4H) |
| 777 | (difluoro(2- (((3S,6S,9aS)-3-(3- (methoxymethyl)-3- phenylazetidine-1- carbonyl)-5- oxooctahydro-1H- pyrrolo[1,2- a]azepin-6- yl)carbamoyl)benzo [b]thiophen-5- yl)methyl) phosphonic acid | 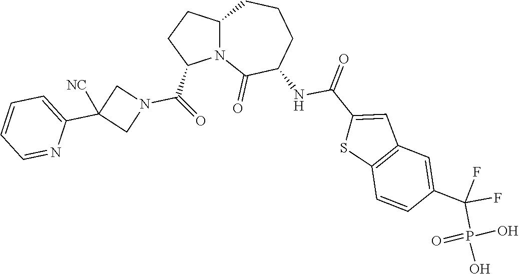 [SMILES: N#CC1(c2ccccn2)CN(C(=O)[C@@H]2CC[C@@H]3CCC[C@H](NC(=O)c4cc5cc(C(F)(F)P(=O)(O)O)ccc5s4)C(=O)N32)C1] | 661.66 | ¹H NMR (400 MHz, DMSO-d₆) δ 8.27 (d, J = 33.4 Hz, 1H), 8.17- 7.99 (m, 2H), 7.57 (t, J = 7.7 Hz, 1H), 7.34 (q, J = 8.4 Hz, 2H), 7.29-7.19 (m, 3H), 4.62 (d, J = 13.2 Hz, 1H), 4.53-4.41 (m, 2H), 4.34 (dd, J = 11.8, 8.5 Hz, 1H), 4.15-3.85 (m, 4H), 3.19 (s, 3H), 2.27- 2.11 (m, 1H), 1.98 (d, J = 31.6 Hz, 2H), 1.88- 1.50 (m, 7H) |
| 778 | (difluoro(2- (((3S,6S,9aS)-5- oxo-3-(3-(o- tolyl)azetidine-1- carbonyl)octahydro- 1H-pyrrolo[1,2- a]azepin-6- yl)carbamoyl)benzo [b]thiophen-5- yl)methyl) phosphonic acid | 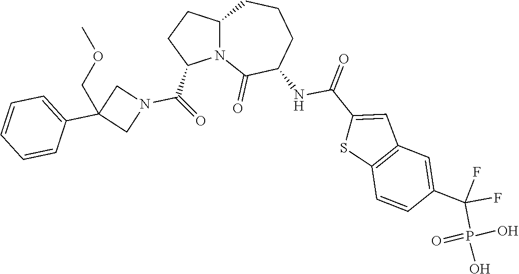 [SMILES: COCC1(c2ccccc2)CN(C(=O)[C@@H]2CC[C@@H]3CCC[C@H](NC(=O)c4cc5cc(C(F)(F)P(=O)(O)O)ccc5s4)C(=O)N32)C1] | 631.63 | ¹H NMR (500 MHz, DMSO-d₆) δ 8.77 (d, J = 7.6 Hz, 1H), 8.28 (d, J = 20.9 Hz, 1H), 8.12-8.03 (m, 2H), 7.60-7.19 (m, 2H), 7.17-6.87 (m, 3H), 4.79-4.55 (m, 2H), 4.44 (dd, J = 7.9, 4.8 Hz, 1H), 4.38-4.22 (m, 2H), 4.19 (d, J = 8.9 Hz, 1H), 4.05 (q, J = 8.3 Hz, 2H), 2.23 (s, 1H), 2.16 (s, 3H), 1.98 (d, J = 40.8 Hz, 2H), 1.88-1.59 (m, 7H) |
| 779 | (difluoro(2- (((3S,6S,9aS)-3-(3- (4-methyl-4H-1,2,4- triazol-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 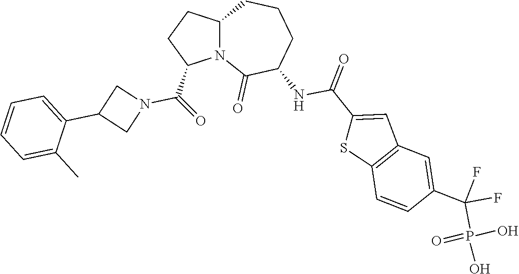 [SMILES: Cc1ccccc1C1CN(C(=O)[C@@H]2CC[C@@H]3CCC[C@H](NC(=O)c4cc5cc(C(F)(F)P(=O)(O)O)ccc5s4)C(=O)N32)C1] | 622.58 | ¹H NMR (600 MHz, DMSO-d₆) δ 8.78 (dd, J = 15.8, 7.7 Hz, 1H), 8.45-8.37 (m, 1H), 8.28 (d, J = 13.8 Hz, 1H), 8.12-8.02 (m, 2H), 7.61-7.51 (m, 1H), 4.80-4.51 (m, 3H), 4.42 (td, J = 8.3, 3.8 Hz, 2H), 4.30- 4.14 (m, 2H), 4.06 (dp, J = 13.2, 6.4, 5.2 Hz, 2H), 3.97 (s, 1H), 2.23-2.13 (m, 1H), 2.02-1.90 (m, 2H), 1.85-1.63 (m, 7H), 1.23-1.10 (m, 1H) |
| 780 | ((2-(((3S,6S,9aS)-3- (3-cyanoazetidine- 1-carbonyl)-5- oxooctahydro-1H- pyrrolo[1,2- a]azepin-6- yl)carbamoyl)benzo [b]thiophen-5- yl)difluoromethyl) phosphonic acid | 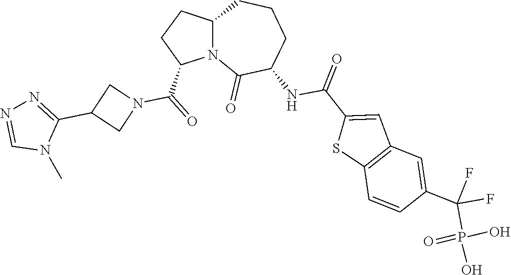 [SMILES: Cn1cnnc1C1CN(C(=O)[C@@H]2CC[C@@H]3CCC[C@H](NC(=O)c4cc5cc(C(F)(F)P(=O)(O)O)ccc5s4)C(=O)N32)C1] | 566.52 | |
| 781 | ((2- (((3S,6S,8R,9aR)-3- ((3S,4R)-3-cyano-4- phenylpyrrolidine- 1-carbonyl)-8- methyl-5- oxooctahydro-1H- pyrrolo[1,2- a]azepin-6- yl)carbamoyl)benzo [b]thiophen-5- yl)difluoromethyl) phosphonic acid Or ((2- (((3S,6S,8S,9aR)-3- ((3S,4R)-3-cyano-4- | 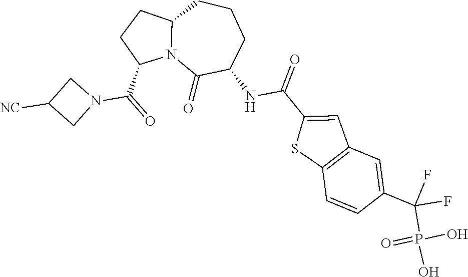 [SMILES: N#CC1CN(C(=O)[C@@H]2CC[C@@H]3CCC[C@H](NC(=O)c4cc5cc(C(F)(F)P(=O)(O)O)ccc5s4)C(=O)N32)C1] | 670.67 | ¹H NMR (500 MHz, DMSO-d₆) δ 8.75 (dd, J = 13.6, 7.6 Hz, 1H), 8.28 (d, J = 11.6 Hz, 1H), 8.11 (dd, J = 8.6, 4.5 Hz, 1H), 8.06 (s, 1H), 7.57 (d, J = 8.6 Hz, 1H), 7.42 (d, J = 8.2 Hz, 2H), 7.38 (td, J = 7.6, 5.7 Hz, 2H), 7.31 (q, J = 7.0 Hz, 1H), 4.65 (ddt, J = 13.3, 8.3, 4.6 Hz, 2H), 4.19-3.87 (m, 6H), 3.27 (t, J = 11.0 Hz, 1H), 2.21 (td, J = 12.0, 11.4, 4.5 Hz, |
| | phenylpyrrolidine- | | | 1H), 2.07 (qd, J = 8.4, |
| | 1-carbonyl)-8- | Or | | 3.6 Hz, 1H), 1.96 (s, |
| | methyl-5- | | | 1H), 1.80 (qt, J = |
| | oxooctahydro-1H- pyrrolo[1,2- a]azepin-6- yl)carbamoyl)benzo [b]thiophen-5- yl)difluoromethyl) phosphonic acid | 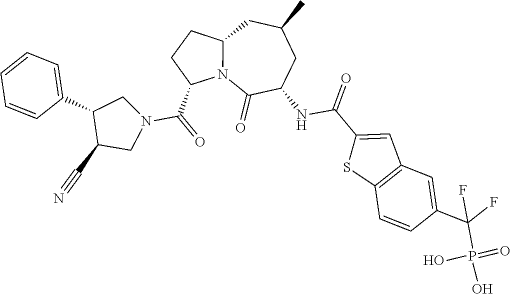 [SMILES: C[C@@H]1C[C@H]2CC[C@@H](C(=O)N3C[C@@H](C#N)[C@H](c4ccccc4)C3)N2C(=O)[C@@H](NC(=O)c2cc3cc(C(F)(F)P(=O)(O)O)ccc3s2)C1] | | 18.3, 6.0 Hz, 3H), 1.69 (d, J = 12.8 Hz, 1H), 1.56 (qd, J = 11.5, 6.3 Hz, 1H), 1.44 (qd, J = 12.0, 4.5 Hz, 1H), 0.95 (dd, J = 6.6, 4.3 Hz, 3H) |
| 782 | ((2- (((3S,6S,8S,9aR)-3- ((3S,4R)-3-cyano-4- phenylpyrrolidine- 1-carbonyl)-8- methyl-5- oxooctahydro-1H- pyrrolo[1,2- a]azepin-6- yl)carbamoyl)benzo [b]thiophen-5- yl)difluoromethyl) phosphonic acid Or ((2- (((3S,6S,8R,9aR)-3- ((3R,4S)-3-cyano-4- phenylpyrrolidine- | 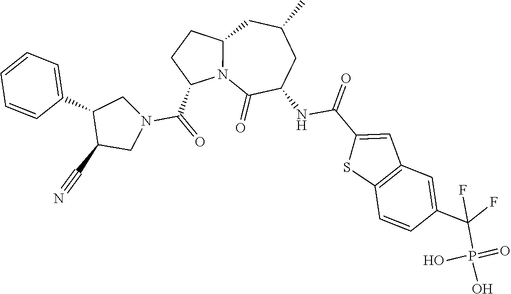 [SMILES: C[C@H]1C[C@H]2CC[C@@H](C(=O)N3C[C@@H](C#N)[C@H](c4ccccc4)C3)N2C(=O)[C@@H](NC(=O)c2cc3cc(C(F)(F)P(=O)(O)O)ccc3s2)C1] | 670.67 | 1H NMR (600 MHz, DMSO-d₆) δ 8.73 (dd, J = 14.9, 7.7 Hz, 1H), 8.27 (d, J = 12.9 Hz, 1H), 8.10 (dd, J = 8.5, 5.9 Hz, 1H), 8.05 (s, 1H), 7.57 (d, J = 8.6 Hz, 1H), 7.42 (d, J = 7.6 Hz, 2H), 7.37 (q, J = 7.5 Hz, 2H), 7.31 (q, J = 7.7 Hz, 1H), 4.89-4.81 (m, 1H), 4.59 (ddd, J = 12.2, 8.1, 4.7 Hz, 1H), 4.17 (q, J = 8.8 Hz, 2H), 4.06 (t, J = 8.9 Hz, 1H), 3.97 (dd, J = 11.4, 8.0 Hz, 1H), |
| | 1-carbonyl)-8- | | | 3.91 (dd, J = 11.8, 8.2 |
| | methyl-5- | | | Hz, 1H), 3.85 (t, J = |
| | oxooctahydro-1H- | | | 10.0 Hz, 1H), 3.08- |
| | pyrrolo[1,2- | | | 3.05 (m, 1H), 2.25 (s, |
| | a]azepin-6- | | | 1H), 2.21-2.14 (m, |
| | yl)carbamoyl)benzo | | | 1H), 2.09 (t, J = 10.3 |
| | [b]thiophen-5- | | | Hz, 1H), 1.97-1.88 |
| | yl)difluoromethyl) | | | (m, 2H), 1.84 (dt, J = |
| | phosphonic acid | | | 12.4, 6.1 Hz, 1H), |
| | | | | 1.78-1.72 (m, 1H), |
| | | | | 1.67 (d, J = 14.9 Hz, |
| | | | | 1H), 1.52-1.45 (m, |
| | | | | 1H), 1.11 (d, J = 7.0 |
| | | | | Hz, 3H) |
| 783 | (difluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 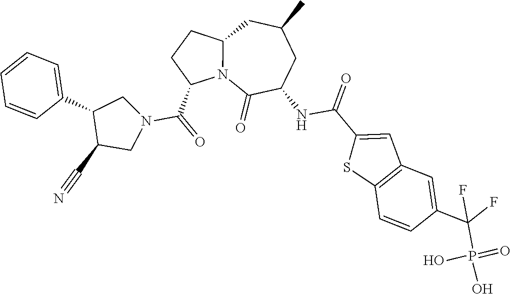 [SMILES: C[C@@H]1C[C@H]2CC[C@@H](C(=O)N3C[C@@H](C#N)[C@H](c4ccccc4)C3)N2C(=O)[C@@H](NC(=O)c2cc3cc(C(F)(F)P(=O)(O)O)ccc3s2)C1] | 632.62 | ¹H NMR (400 MHz, DMSO-d₆) δ 8.79- 8.59 (m, 1H), 8.43- 8.11 (m, 3H), 8.11- 7.95 (m, 2H), 7.55 (dd, J = 17.2, 8.6 Hz, 1H), 7.28 (dd, J = 37.3, 5.1 Hz, 1H), 4.84-4.60 (m, 2H), 4.48-4.24 (m, 3H), 4.10-3.96 (m, 4H), 3.89 (d, J = 2.7 Hz, 1H), 2.20 (d, J = 36.4 Hz, 3H), 1.98 (q, J = 10.1, 5.8 Hz, 2H), 1.88-1.65 (m, 6H) |
| 784 | (difluoro(2- (((3S,6S,9aS)-3-(3- isopropyl-3- phenylazetidine-1- carbonyl)-5- oxooctahydro-1H- pyrrolo[1,2- a]azepin-6- yl)carbamoyl)benzo [b]thiophen-5- yl)methyl) phosphonic acid | 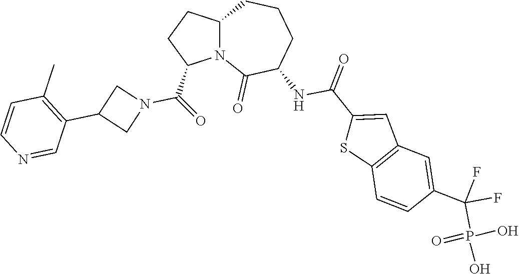 [SMILES: Cc1ccncc1C1CN(C(=O)[C@@H]2CC[C@@H]3CCC[C@H](NC(=O)c4cc5cc(C(F)(F)P(=O)(O)O)ccc5s4)C(=O)N32)C1] | 659.69 | ¹H NMR (500 MHz, DMSO-d₆) δ 8.86- 8.16 (m, 1H), 8.16- 7.97 (m, 3H), 7.61- 7.08 (m, 5H), 6.92 (d, J = 8.9 Hz, 1H), 4.61 (s, 1H), 4.54-4.29 (m, 3H), 4.05-3.92 (m, 3H), 3.15 (s, 1H), 1.92 (s, 4H), 1.74 (d, J = 49.7 Hz, 6H), 0.71 (dd, J = 19.1, 7.1 Hz, 6H) |
| 785 | (difluoro(2- (((3S,6S,9aS)-3-(3- (oxetan-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 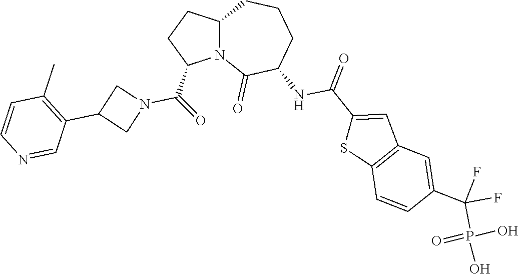 [SMILES: Cc1ccncc1C1CN(C(=O)[C@@H]2CC[C@@H]3CCC[C@H](NC(=O)c4cc5cc(C(F)(F)P(=O)(O)O)ccc5s4)C(=O)N32)C1] | 597.57 | |
| 786 | (difluoro(2- (((3S,6S,10aS)-5- oxo-3-(4- azaspiro[2.4] heptane-4- carbonyl)decahydro pyrrolo[1,2- a]azocin-6- yl)carbamoyl)benzo [b]thiophen-5- yl)methyl) phosphonic acid | 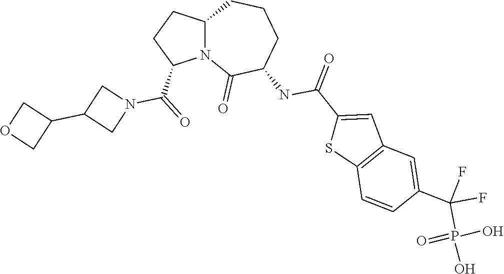 | 595.6 | |
| 787 | (difluoro(2- (((3S,6S,10aS)-3-(3- (methylsulfonamido) azetidine-1- carbonyl)-5- oxodecahydropyrrolo [1,2-a]azocin-6- yl)carbamoyl)benzo [b]thiophen-5- yl)methyl) phosphonic acid | 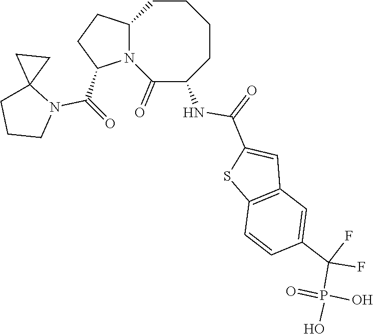 [SMILES: O=C(N[C@H]1CCCC[C@H]2CC[C@@H](C(=O)N3CCCC34CC4)N2C1=O)c1cc2cc(C(F)(F)P(=O)(O)O)ccc2s1] | 648.63 | ¹H NMR (500 MHz, DMSO-d₆) δ 8.83 (dd, J = 10.4, 7.0 Hz, 1H), 8.30 (d, J = 1.8 Hz, 1H), 8.12 (d, J = 8.5 Hz, 1H), 8.06 (s, 1H), 7.86 (dd, J = 25.6, 7.0 Hz, 1H), 7.57 (d, J = 9.0 Hz, 1H), 4.95-4.88 (m, 1H), 4.53 (dt, J = 128.7, 7.8 Hz, 1H), 4.22 (dd, J = 12.3, 5.6 Hz, 3H), 4.07-4.00 (m, 2H), 2.89 (d, J = 8.0 Hz, 3H), 2.12 (dd, J = 12.0, 7.2 Hz, 1H), 2.07-1.97 (m, 1H), 1.90-1.77 (m, 7H), 1.72 (dd, J = 12.0, 5.5 |
| | | | | Hz, 1H), 1.62 (s, 1H), |
| | | | | 1.55 (d, J = 12.9 Hz, |
| | | | | 1H), 1.49 (s, 1H). |
| 788 | (difluoro(2- (((3S,6S,10aS)-3- (methyl(2-(2-oxo- 1,2-dihydropyridin- 4-yl)ethyl) carbamoyl)-5- oxodecahydropyrrolo [1,2-a]azocin-6- yl)carbamoyl)benzo [b]thiophen-5- yl)methyl) phosphonic acid | 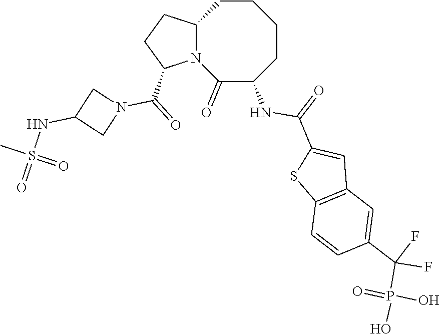 [SMILES: CS(=O)(=O)NC1CN(C(=O)[C@@H]2CC[C@@H]3CCCC[C@H](NC(=O)c4cc5cc(C(F)(F)P(=O)(O)O)ccc5s4)C(=O)N32)C1] | 650.63 | ¹H NMR (600 MHz, DMSO-d₆) δ 11.48 (s, 1H), 8.83 (dd, J = 11.7, 7.2 Hz, 1H), 8.30 (t, J = 3.6 Hz, 1H), 8.25-8.10 (m, 3H), 8.05 (s, 1H), 7.56 (d, J = 8.6 Hz, 1H), 7.26 (d, J = 6.7 Hz, 1H), 6.23-6.10 (m, 2H), 4.99-4.91 (m, 1H), 4.72-4.66 (m, 1H), 3.02 (d, J = 4.3 Hz, 3H), 2.75 (d, J = 2.5 Hz, 2H), 2.73 (d, J = 8.0 Hz, 1H), 2.12-1.98 (m, 4H), 1.87 (d, J = 14.7 Hz, 2H), 1.82 (d, J = 14.5 Hz, 2H), 1.70 (d, J = |
| | | | | 13.5 Hz, 1H), 1.62 (d, |
| | | | | J = 14.3 Hz, 1H), 1.53 |
| | | | | (d, J = 14.4 Hz, 2H) |
| 789 | ((2-(((3S,6S,10aS)- 3-(2,3-dihydro-1H- pyrrolo[3,4- c]pyridine-2- carbonyl)-5- oxodecahydropyrrolo [1,2-a]azocin-6- yl)carbamoyl)benzo [b]thiophen-5- yl)difluoromethyl) phosphonic acid | 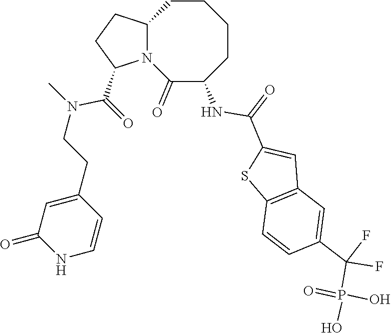 [SMILES: CN(CCc1cc[nH]c(=O)c1)C(=O)[C@@H]1CC[C@@H]2CCCC[C@H](NC(=O)c3cc4cc(C(F)(F)P(=O)(O)O)ccc4s3)C(=O)N21] | 618.59 | ¹H NMR (400 MHz, DMSO-d₆) δ 8.61 8.15 (m, 2H), 8.11 (d, J = 8.8 Hz, 2H), 7.98 (d, J = 38.7 Hz, 1H), 7.52 (dd, J = 18.9, 7.5 Hz, 1H), 6.93 (d, J = 8.7 Hz, 2H), 5.26- 4.76 (m, 3H), 4.72 (d, J = 18.0 Hz, 1H), 4.66- 4.29 (m, 3H), 2.93 (d, J = 105.2 Hz, 2H), 2.41 (s, 2H), 2.09 (s, 1H), 1.98-1.79 (m, 5H), 1.62 (t, J = 26.5 Hz, 2H) |
| 790 | (difluoro(2- (((3S,6S,9aS)-3-(3- ((methylsulfonyl) methyl)azetidine-1- carbonyl)-5- oxooctahydro-1H- pyrrolo[1,2- a]azepin-6- yl)carbamoyl)benzo [b]thiophen-5- yl)methyl) phosphonic acid | 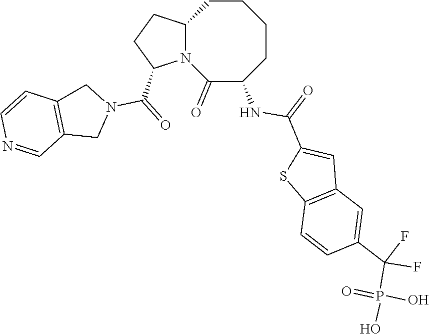 [SMILES: O=C(N[C@H]1CCCC[C@H]2CC[C@@H](C(=O)N3Cc4ccncc4C3)N2C1=O)c1cc2cc(C(F)(F)P(=O)(O)O)ccc2s1] | 633.62 | ¹H NMR (500 MHz, DMSO-d₆) δ 8.75 (dd, J = 7.6, 4.0 Hz, 1H), 8.28 (s, 1H), 8.12 (d, J = 8.5 Hz, 1H), 8.06 (s, 1H), 7.57 (d, J = 8.6 Hz, 1H), 4.63 (s, 1H), 4.52-4.14 (m, 4H), 4.04-3.96 (m, 3H), 3.17-3.04 (m, 2H), 2.95 (s, 3H), 2.16 (s, 1H), 1.96 (d, J = 11.6 Hz, 2H), 1.84 (d, J = 13.2 Hz, 1H), 1.80- 1.60 (m, 6H) |
| 791 | (difluoro(2- (((3S,6S,10aS)-3- (methyl(1-(2-oxo- 1,2-dihydropyridin- 4-yl)ethyl) carbamoyl)-5- oxodecahydropyrrolo [1,2-a]azocin-6- yl)carbamoyl)benzo [b]thiophen-5- yl)methyl) phosphonic acid | 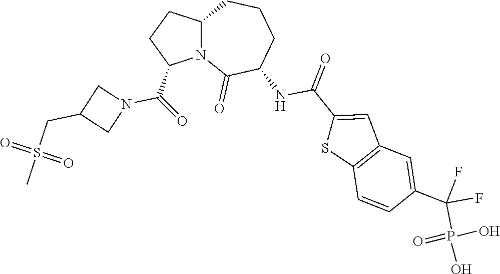 [SMILES: CS(=O)(=O)CC1CN(C(=O)[C@@H]2CC[C@@H]3CCC[C@H](NC(=O)c4cc5cc(C(F)(F)P(=O)(O)O)ccc5s4)C(=O)N32)C1] | 650.63 | |
| 792 | (difluoro(2- (((3S,6S,10aS)-5- oxo-3-((1-(2-oxo- 1,2-dihydropyridin- 4-yl)ethyl)carbamoyl) decahydropyrrolo[1, 2-a]azocin-6- yl)carbamoyl)benzo [b]thiophen-5- yl)methyl) phosphonic acid | 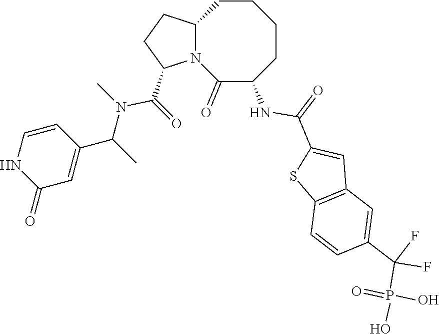 [SMILES: CC(c1cc[nH]c(=O)c1)N(C)C(=O)[C@@H]1CC[C@@H]2CCCC[C@H](NC(=O)c3cc4cc(C(F)(F)P(=O)(O)O)ccc4s3)C(=O)N21] | 636.61 | ¹H NMR (600 MHz, DMSO-d₆) δ 8.87 (dd, J = 56.1, 7.0 Hz, 1H), 8.39-8.35 (m, 1H), 8.31 (d, J = 6.5 Hz, 1H), 8.22-8.04 (m, 3H), 7.57 (d, J = 8.7 Hz, 1H), 7.27 (dd, J = 21.7, 6.8 Hz, 1H), 6.28-6.16 (m, 1H), 6.15-6.08 (m, 1H), 4.91 (td, J = 12.1, 6.3 Hz, 1H), 4.67-4.62 (m, 1H), 4.35 (dd, J = 16.1, 7.9 Hz, 1H), 4.25 (d, J = 7.7 Hz, 1H), 2.12 (t, J = 9.6 Hz, 1H), 2.04 (dd, J = 14.0, 6.8 Hz, 1H), 1.86-1.80 (m, 3H), |
| | | | | 1.79-1.74 (m, 2H), |
| | | | | 1.70 (dd, J = 12.2, 7.5 |
| | | | | Hz, 2H), 1.63-1.58 |
| | | | | (m, 1H), 1.57-1.53 |
| | | | | (m, 1H), 1.48 (d, J = |
| | | | | 11.6 Hz, 1H), 1.26 |
| | | | | (dd, J = 7.1, 2.5 Hz, |
| | | | | 3H) |
| 793 | (difluoro(2- (((3S,6S,10aS)-3- ((1-isopropyl-1H- pyrazol-4- yl)(methyl) carbamoyl)-5- oxodecahydropyrrol o[1,2-a]azocin-6- yl)carbamoyl)benzo [b]thiophen-5- yl)methyl) phosphonic acid | 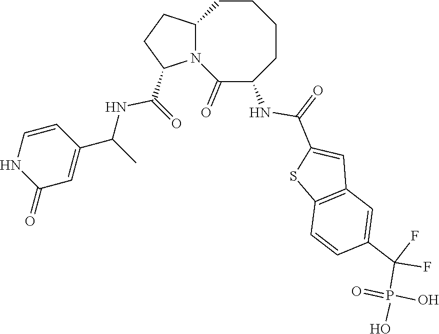 [SMILES: CC(NC(=O)[C@@H]1CC[C@@H]2CCCC[C@H](NC(=O)c3cc4cc(C(F)(F)P(=O)(O)O)ccc4s3)C(=O)N21)c1cc[nH]c(=O)c1] | 637.64 | |
| 794 | ((2-(((3S,6S,10aS)- 3-(2-cyclopropyl- 2,4,5,6- tetrahydropyrrolo[3, 4-c]pyrazole-5- carbonyl)-5- oxodecahydropyrrolo [1,2-a]azocin-6- yl)carbamoyl)benzo [b]thiophen-5- yl)difluoromethyl) phosphonic acid | 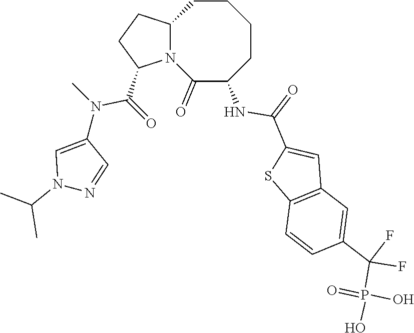 [SMILES: CC(C)n1cc(N(C)C(=O)[C@@H]2CC[C@@H]3CCCC[C@H](NC(=O)c4cc5cc(C(F)(F)P(=O)(O)O)ccc5s4)C(=O)N32)cn1] | 647.63 | ¹H NMR (400 MHz, DMSO-d₆) δ 8.70 (s, 1H), 8.28 (s, 1H), 8.10 (d, J = 8.6 Hz, 1H), 8.05 (s, 1H), 7.58 (d, J = 8.2 Hz, 2H), 4.96 (dd, J = 15.6, 7.1 Hz, 1H), 4.87-4.78 (m, 1H), 4.60 (t, J = 9.5 Hz, 2H), 4.35 (dq, J = 28.2, 12.6 Hz, 4H), 2.29 (d, J = 10.4 Hz, 1H), 2.09 (s, 1H), 1.98-1.73 (m, 7H), 1.68-1.47 (m, 3H), 1.01 (s, 2H), 0.93 (d, J = 7.1 Hz, 2H) |
| 795 | (difluoro(2- (((3S,6S,10aS)-3- (methyl((2-oxo-1,2- dihydroquinolin-4- yl)methyl) carbamoyl)-5- oxodecahydropyrrolo [1,2-a]azocin-6- yl)carbamoyl)benzo [b]thiophen-5- yl)methyl) phosphonic acid | 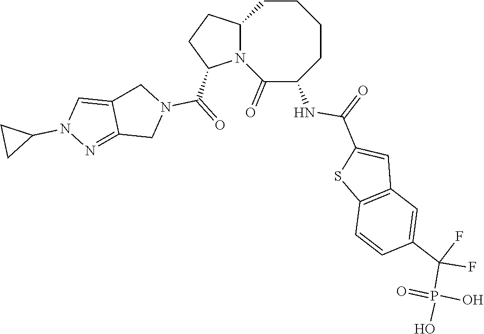 [SMILES: O=C(N[C@H]1CCCC[C@H]2CC[C@@H](C(=O)N3Cc4cn(C5CC5)nc4C3)N2C1=O)c1cc2cc(C(F)(F)P(=O)(O)O)ccc2s1] | 686.67 | ¹H NMR (500 MHz, DMSO-d₆) δ 8.95- 8.90 (m, 1H), 8.33 (s, 1H), 8.11 (d, J = 9.0 Hz, 1H), 8.05 (s, 2H), 7.71 (d, J = 8.0 Hz, 1H), 7.57 (d, J = 8.2 Hz, 1H), 7.50 (t, J = 7.5 Hz, 1H), 7.31 (d, J = 7.8 Hz, 1H), 7.16 (t, J = 7.7 Hz, 1H), 6.25 (d, J = 6.1 Hz, 1H), 4.96 (s, 1H), 4.93-4.87 (m, 1H), 4.75 (s, 2H), 4.32 (s, 1H), 3.13 (s, 3H), 2.10 (d, J = 8.1 Hz, 2H), 1.87 (s, 4H), 1.79 (q, J = 9.2, 8.5 Hz, 4H), 1.58 (s, 2H) |
| 796 | (difluoro(2- (((3S,6S,9aS)-3-(3- methoxy-3-(4- methyl-4H-1,2,4- triazol-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 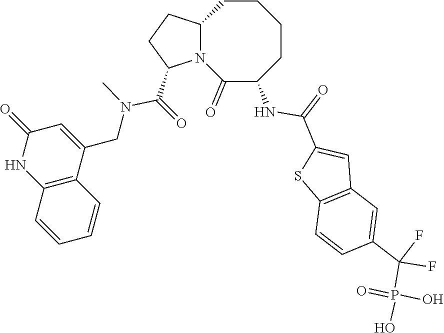 [SMILES: CN(Cc1cc(=O)[nH]c2ccccc12)C(=O)[C@@H]1CC[C@@H]2CCCC[C@H](NC(=O)c3cc4cc(C(F)(F)P(=O)(O)O)ccc4s3)C(=O)N21] | 652.61 | ¹H NMR (400 MHz, DMSO-d₆) δ 8.82 (dd, J = 21.1, 7.6 Hz, 1H), 8.62 (s, 1H), 8.30 (d, J = 23.3 Hz, 1H), 8.13 (t, J = 8.3 Hz, 1H), 8.08 (d, J = 7.2 Hz, 1H), 7.58 (t, J = 7.2 Hz, 1H), 4.90- 4.80 (m, 1H), 4.66 (q, J = 9.9 Hz, 2H), 4.51 (d, J = 8.6 Hz, 1H), 4.45 (d, J = 5.6 Hz, 1H), 4.12 (t, J = 9.2 Hz, 1H), 3.99 (s, 1H), 3.59 (s, 3H), 2.98 (d, J = 11.3 Hz, 3H), 2.25- 2.14 (m, 1H), 2.08- 1.89 (m, 2H), 1.86- 1.64 (m, 6H), 1.29- 1.07 (m, 1H) |
| 797 | (difluoro(2- (((3S,6S,9aS)-5- oxo-3-(3-(pyrazin- 2-yl)azetidine-1- carbonyl)octahydro- 1H-pyrrolo[1,2- a]azepin-6- yl)carbamoyl)benzo [b]thiophen-5- yl)methyl) phosphonic acid | 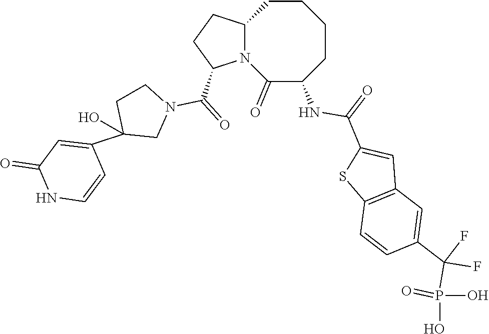 [SMILES: O=C(N[C@H]1CCCC[C@H]2CC[C@@H](C(=O)N3CCC(O)(c4cc[nH]c(=O)c4)C3)N2C1=O)c1cc2cc(C(F)(F)P(=O)(O)O)ccc2s1] | 619.58 | ¹H NMR (600 MHz, DMSO-d₆) δ 8.77 (d, J = 7.6 Hz, 1H), 8.69- 8.60 (m, 2H), 8.53 (dd, J = 16.1, 2.5 Hz, 1H), 8.29 (d, J = 14.1 Hz, 1H), 8.10 (dd, J = 8.5, 4.7 Hz, 1H), 8.06 (d, J = 6.3 Hz, 1H), 7.57 (d, J = 8.3 Hz, 1H), 4.69 (dt, J = 52.3, 9.2 Hz, 1H), 4.58-4.41 (m, 2H), 4.33-4.21 (m, 1H), 4.18-4.03 (m, 2H), 3.96 (s, 1H), 3.06 (p, J = 6.9 Hz, 1H), 2.19 (dd, J = 12.5, 6.0 Hz, 1H), 2.07-1.97 (m, 1H), 1.94 (s, 1H), |
| | | | | 1.85-1.65 (m, 7H) |
| 798 | (difluoro(2- (((3S,6S,10aS)-5- oxo-3-(4,5,6,7- tetrahydro-1H- pyrazolo[3,4- c]pyridine-6- carbonyl)decahydro pyrrolo[1,2- a]azocin-6- yl)carbamoyl)benzo [b]thiophen-5- yl)methyl) phosphonic acid | 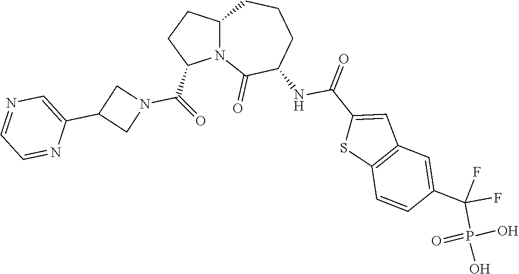 [SMILES: O=C(N[C@H]1CCC[C@H]2CC[C@@H](C(=O)N3CC(c4cnccn4)C3)N2C1=O)c1cc2cc(C(F)(F)P(=O)(O)O)ccc2s1] | 621.6 | ¹H NMR (600 MHz, DMSO-d₆) δ 8.76 (d, J = 7.4 Hz, 1H), 8.24 (s, 1H), 8.06-7.94 (m, 2H), 7.57 (d, J = 8.6 Hz, 1H), 7.41 (d, J= 14.1 Hz, 1H), 7.17 (s, 1H), 4.91 (tt, J = 25.5, 7.0 Hz, 2H), 4.74 (dd, J = 15.8, 7.0 Hz, 1H), 4.41-4.23 (m, 2H), 3.91-3.67 (m, 2H), 3.63 (p, J = 4.8 Hz, 1H), 2.79 (dd, J = 14.9, 8.2 Hz, 1H), 2.29-2.18 (m, 1H), 2.13-2.04 (m, 2H), 1.94-1.66 (m, 6H), |
| | | | | 1.61 (s, 1H), 1.54 (d, |
| | | | | J = 16.7 Hz, 1H), 1.52- |
| | | | | 1.42 (m, 1H) |
| 799 | (difluoro(2- (((3S,6S,9aS)-3-(3- (1-methyl-1H- imidazol-2- yl)azetidine-1- carbonyl)-5- oxooctahydro-1H- pyrrolo[1,2- a]azepin-6- yl)carbamoyl)benzo [b]thiophen-5- yl)methyl) phosphonic acid | 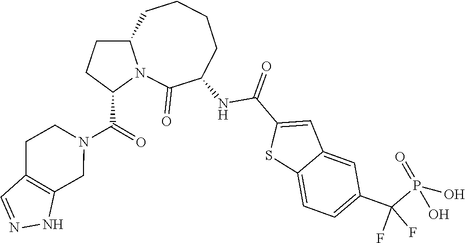 [SMILES: O=C(N[C@H]1CCCC[C@H]2CC[C@@H](C(=O)N3CCc4cn[nH]c4C3)N2C1=O)c1cc2cc(C(F)(F)P(=O)(O)O)ccc2s1] | 621.6 | ¹H NMR (400 MHz, DMSO-d₆) δ 8.75 (d, J = 7.6 Hz, 1H), 8.23 (d, J = 26.3 Hz, 1H), 8.03 (t, J = 11.4 Hz, 2H), 7.57 (t, J = 7.7 Hz, 1H), 7.28 (d, J = 36.5 Hz, 1H), 7.09 (d, J= 55.5 Hz, 1H), 4.77- 4.53 (m, 2H), 4.51- 4.37 (m, 2H), 4.24- 4.12 (m, 2H), 4.08 (dd, J = 16.9, 8.1 Hz, 1H), 3.98 (s, 1H), 3.59 (s, 3H), 2.18 (s, |
| | | | | 1H), 2.07-1.92 (m, |
| | | | | 2H), 1.89-1.64 (m, |
| | | | | 7H |
| 800 | (difluoro(2- (((3S,6S,10aS)-3- (methyl((2-oxo-1,2- dihydropyridin-4- yl)methyl) carbamoyl)-5- oxodecahydropyrrolo [1,2-a]azocin-6- yl)carbamoyl)benzo [b]thiophen-5- yl)methyl) phosphonic acid | 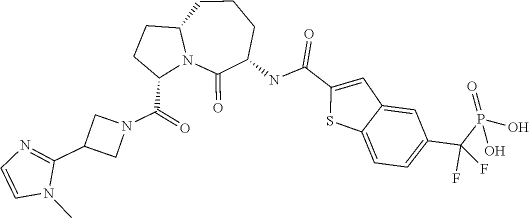 | 636.61 | ¹H NMR (600 MHz, DMSO-d₆) δ 11.41 (s, 1H), 8.89 (dd, J = 24.7, 7.1 Hz, 1H), 8.31 (d, J = 8.3 Hz, 1H), 8.09 (t, J = 7.0 Hz, 1H), 8.04 (s, 1H), 7.57 (d, J = 8.8 Hz, 1H), 7.32-7.23 (m, 1H), 6.18-5.98 (m, 2H), 4.96 (dt, J = 12.1, 5.6 Hz, 1H), 4.80 (t, J = 8.4 Hz, |
| | | | | 1H), 4.47 (d, J = 15.7 |
| | | | | Hz, 1H), 4.17 (d, J = |
| | | | | 16.0 Hz, 1H), 3.02 (s, |
| | | | | 3H), 2.76 (s, 1H), |
| | | | | 2.08 (d, J = 18.3 Hz, |
| | | | | 2H), 1.93-1.68 (m, |
| | | | | 6H), 1.67-1.42 (m, |
| | | | | 4H) |
| 801 | ((2-(((3S,6S,10aS)- 3-(6,7-dihydro-5H- pyrrolo[3,4- b]pyridine-6- carbonyl)-5- oxodecahydropyrrolo [1,2-a]azocin-6- yl)carbamoyl)benzo [b]thiophen-5- yl)difluoromethyl) phosphonic acid | 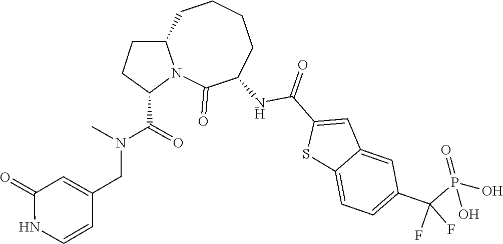 [SMILES: CN(Cc1cc[nH]c(=O)c1)C(=O)[C@@H]1CC[C@@H]2CCCC[C@H](NC(=O)c3cc4cc(C(F)(F)P(=O)(O)O)ccc4s3)C(=O)N21] | 618.59 | ¹H NMR (500 MHz, DMSO-d₆) δ 8.80 (t, J = 7.5 Hz, 1H), 8.44 (dd, J = 9.4, 4.9 Hz, 1H), 8.27 (s, 1H), 8.10 (d, J = 8.5 Hz, 1H), 8.02 (s, 1H), 7.76 (dd, J = 24.7, 7.8 Hz, 1H), 7.55 (d, J = 8.6 Hz, 1H), 7.30 (t, J = 6.7 Hz, 1H), 4.99- 4.89 (m, 2H), 4.74- 4.55 (m, 4H), 4.29 (t, J = 9.9 Hz, 1H), 2.31 (s, 1H), 2.09 (s, 1H), 1.94-1.87 (m, 4H), |
| | | | | 1.79 (d, J = 12.9 Hz, |
| | | | | 3H), 1.64 (s, 1H), |
| | | | | 1.59 (d, J = 15.1 Hz, |
| | | | | 1H), 1.52 (d, J = 11.2 |
| | | | | Hz, 1H) |
| 802 | (difluoro(2- (((3S,6S,10aS)-5- oxo-3-(4,5,6,7- tetrahydro-1H- pyrazolo[4,3- c]pyridine-5- carbonyl)decahydro pyrrolo[1,2- a]azocin-6- yl)carbamoyl)benzo [b]thiophen-5- yl)methyl) phosphonic acid | 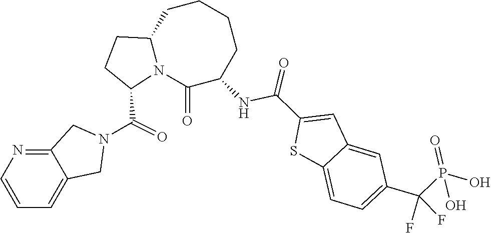 [SMILES: O=C(N[C@H]1CCCC[C@H]2CC[C@@H](C(=O)N3Cc4cccnc4C3)N2C1=O)c1cc2cc(C(F)(F)P(=O)(O)O)ccc2s1] | 621.6 | ¹H NMR (500 MHz, DMSO-d₆) δ 8.79 (s, 1H), 8.27 (s, 1H), 8.12-8.02 (m, 3H), 7.59 (dd, J = 39.0, 8.6 Hz, 2H), 7.37 (d, J = 12.2 Hz, 1H), 4.98- 4.89 (m, 2H), 4.70 (t, J = 14.4 Hz, 1H), 4.26 (s, 2H), 3.94 (d, J = 14.8 Hz, 1H), 2.25 (s, 1H), 2.07 (s, 2H), 1.88 (d, J = 14.9 Hz, 1H), 1.75 (d, J = 20.6 |
| | | | | Hz, 4H), 1.56 (t, J = |
| | | | | 16.6 Hz, 4H), 1.23 (t, |
| | | | | J = 6.8 Hz, 2H) |
| 803 | (difluoro(2- (((3S,6S,10aS)-5- oxo-3-(3-(2-oxo- 1,2-dihydropyridin- 3-yl)azetidine-1- carbonyl)decahydro pyrrolo[1,2- a]azocin-6- yl)carbamoyl)benzo [b]thiophen-5- yl)methyl) phosphonic acid | 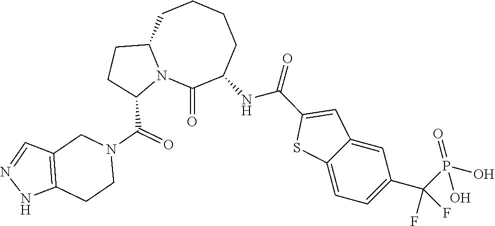 [SMILES: O=C(N[C@H]1CCCC[C@H]2CC[C@@H](C(=O)N3CCc4[nH]ncc4C3)N2C1=O)c1cc2cc(C(F)(F)P(=O)(O)O)ccc2s1] | 648.62 | ¹H NMR (500 MHz, DMSO-d₆) δ 8.83 (d, J = 7.8 Hz, 1H), 8.30 (d, J = 10.8 Hz, 1H), 8.14 (d, J = 17.3 Hz, 1H), 8.09 (d, J = 9.8 Hz, 2H), 8.04 (s, 1H), 7.56 (d, J = 8.9 Hz, 1H), 7.41 (dd, J = 28.8, 6.7 Hz, 1H), 7.26 (dd, J = 14.8, 6.3 Hz, 1H), 4.92 (s, 1H), 4.28 (d, J = 9.2 Hz, 2H), 4.24 (s, 2H), |
| | | | | 4.15 (d, J = 7.2 Hz, |
| | | | | 1H), 2.13 (s, 1H), |
| | | | | 2.01 (s, 1H), 1.82 (s, |
| | | | | 4H), 1.69 (d, J = 11.4 |
| | | | | Hz, 2H), 1.56 (s, 1H), |
| | | | | 1.48 (s, 1H), 1.13 (t, |
| | | | | J = 7.3 Hz, 4H) |
| 804 | (difluoro(2- (((3S,6S,10aS)-3- ((1R,5S,6S)-6-(5- methyl-1,3,4- oxadiazol-2-yl)-3- azabicyclo[3.1.0] hexane-3- carbonyl)-5- oxodecahydropyrrolo [1,2-a]azocin-6- yl)carbamoyl)benzo [b]thiophen-5- yl)methyl)phosphon ic acid | 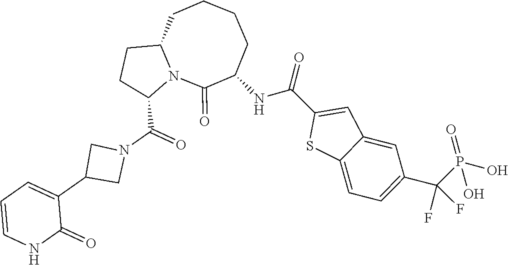 [SMILES: O=C(N[C@H]1CCCC[C@H]2CC[C@@H](C(=O)N3CC(c4ccc[nH]c4=O)C3)N2C1=O)c1cc2cc(C(F)(F)P(=O)(O)O)ccc2s1] | 663.63 | |
| 805 | (difluoro(2- (((3S,6S,10aS)-5- oxo-3-((1R,5S,6R)- 6-phenyl-3- azabicyclo[3.1.0] hexane-3- carbonyl)decahydro pyrrolo[1,2- a]azocin-6- yl)carbamoyl)benzo [b]thiophen-5- yl)methyl) phosphonic acid | 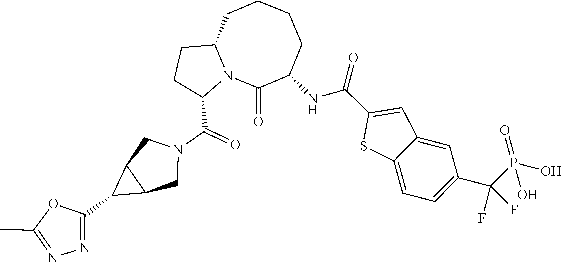 [SMILES: Cc1nnc([C@H]2[C@@H]3CN(C(=O)[C@@H]4CC[C@@H]5CCCC[C@H](NC(=O)c6cc7cc(C(F)(F)P(=O)(O)O)ccc7s6)C(=O)N54)C[C@@H]32)o1] | 657.67 | ¹H NMR (400 MHz, DMSO-d₆) δ 8.84 (t, J = 9.3 Hz, 1H), 8.32 (d, J = 3.5 Hz, 1H), 8.12 (d, J = 8.3 Hz, 1H), 8.06 (s, 1H), 7.58 (d, J = 8.4 Hz, 1H), 7.29-7.19 (m, 2H), 7.14 (d, J = 5.8 Hz, 1H), 7.07 (dd, J = 13.4, 7.7 Hz, 2H), 4.95 (s, 1H), 4.52 (dd, J = 17.9, 9.2 Hz, 1H), 4.27 (s, 1H), 4.04 (d, J = 10.3 Hz, 1H), 3.90- 3.78 (m, 2H), 3.67 |
| | | | | (dd, J = 20.5, 11.6 |
| | | | | Hz, 2H), 3.40 (td, J = |
| | | | | 12.9, 12.0, 7.0 Hz, |
| | | | | 2H), 2.29-2.16 (m, |
| | | | | 1H), 2.08 (d, J = 5.5 |
| | | | | Hz, 1H), 1.99 (d, J = |
| | | | | 5.9 Hz, 2H), 1.92- |
| | | | | 1.74 (m, 6H), 1.66 |
| | | | | 1.53 (m, 2H) |
| 806 | ((2-(((3S,6S,10aS)- 3-(3-(5- cyanopyrimidin-2- yl)azetidine-1- carbonyl)-5- oxodecahydropyrrolo [1,2-a]azocin-6- yl)carbamoyl)benzo [b]thiophen-5- yl)difluoromethyl) phosphonic acid | 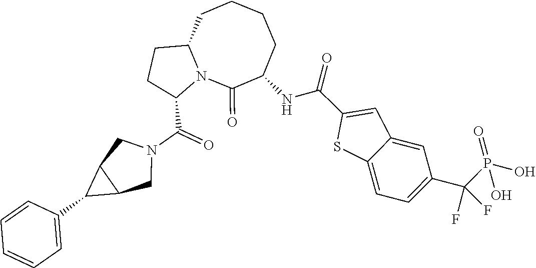 [SMILES: O=C(N[C@H]1CCCC[C@H]2CC[C@@H](C(=O)N3C[C@@H]4[C@H](C3)[C@H]4c3ccccc3)N2C1=O)c1cc2cc(C(F)(F)P(=O)(O)O)ccc2s1] | 658.62 | ¹H NMR (400 MHz, DMSO-d₆) δ 9.30 (d, J = 11.8 Hz, 1H), 8.87 (s, 1H), 8.32 (d, J = 11.6 Hz, 1H), 8.13 (d, J = 9.0 Hz, 1H), 8.06 (s, 1H), 7.58 (d, J = 8.9 Hz, 1H), 5.15- 4.38 (m, 3H), 4.37- 3.95 (m, 5H), 2.03 (s, 2H), 1.84 (s, 7H), 1.57 (d, J = 16.0 Hz, 3H) |
| 807 | (difluoro(2- (((3S,6S,9aS)-3-(3- hydroxy-3-(4- methyl-4H-1,2,4- triazol-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 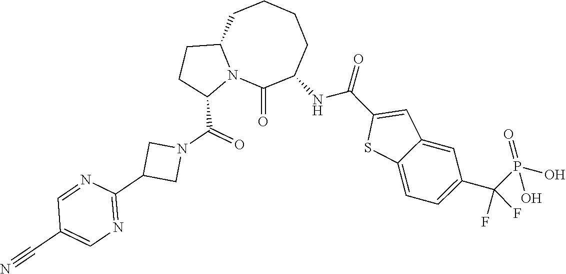 [SMILES: N#Cc1cnc(C2CN(C(=O)[C@@H]3CC[C@@H]4CCCC[C@H](NC(=O)c5cc6cc(C(F)(F)P(=O)(O)O)ccc6s5)C(=O)N43)C2)nc1] | 638.58 | ¹H NMR (500 MHz, DMSO-d₆) δ 8.77 (d, J = 7.6 Hz, 1H), 8.46 (d, J = 13.6 Hz, 1H), 8.27 (d, J = 21.9 Hz, 1H), 8.11 (t, J = 9.1 Hz, 1H), 8.06 (d, J = 9.9 Hz, 1H), 7.56 (t, J = 7.8 Hz, 1H), 6.88 (d, J = 14.7 Hz, 1H), 4.89 (dd, J = 27.9, 9.1 Hz, 1H), 4.63 (q, J = 9.6 Hz, 1H), 4.53 (dd, J = 23.5, 9.6 Hz, 1H), 4.48-4.31 (m, 2H), 4.00 (dd, J = 25.7, 9.9 Hz, 2H), 3.61 (d, J = |
| | | | | 3.0 Hz, 3H), 2.23- |
| | | | | 2.09 (m, 1H), 2.06- |
| | | | | 1.86 (m, 2H), 1.87- |
| | | | | 1.53 (m, 7H) |
| 808 | ((2-(((3S,6S,9aS)-3- (3-ethoxy-3-(4- methyl-4H-1,2,4- triazol-3- yl)azetidine-1- carbonyl)-5- oxooctahydro-1H- pyrrolo[1,2- a]azepin-6- yl)carbamoyl)benzo [b]thiophen-5- yl)difluoromethyl) phosphonic acid | 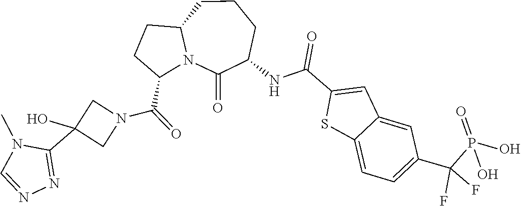 [SMILES: Cn1cnnc1C1(O)CN(C(=O)[C@@H]2CC[C@@H]3CCC[C@H](NC(=O)c4cc5cc(C(F)(F)P(=O)(O)O)ccc5s4)C(=O)N32)C1] | 666.64 | ¹H NMR (400 MHz, DMSO-d₆) δ 8.82 (dd, J = 19.0, 7.2 Hz, 1H), 8.54 (d, J = 12.9 Hz, 1H), 8.28 (dd, J = 22.0, 3.2 Hz, 1H), 8.16-8.09 (m, 1H), 8.07 (s, 1H), 7.57 (s, 1H), 4.85 (dd, J = 28.4, 9.2 Hz, 1H), 4.63 (d, J = 10.9 Hz, 1H), 4.50 (d, J = 9.3 Hz, 1H), 4.46-4.41 |
| | | | | (m, 1H), 4.12 (d, J = |
| | | | | 9.6 Hz, 1H), 3.98 (s, |
| | | | | 1H), 3.58 (t, J = 2.3 |
| | | | | Hz, 3H), 3.19-3.03 |
| | | | | (m, 2H), 2.18 (s, 1H), |
| | | | | 2.06-1.91 (m, 2H), |
| | | | | 1.71 (s, 8H), 1.13- |
| | | | | 1.01 (m, 3H) |
| 809 | (difluoro(2- (((3S,6S,9aS)-3-(3-(4- hydroxypyrimidin- 2-yl)azetidine-1- carbonyl)-5- oxooctahydro-1H- pyrrolo[1,2- a]azepin-6- yl)carbamoyl)benzo [b]thiophen-5- yl)methyl) phosphonic acid | 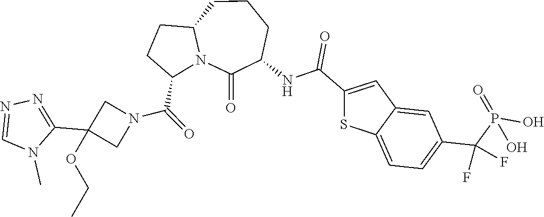 [SMILES: CCOC1(c2nncn2C)CN(C(=O)[C@@H]2CC[C@@H]3CCC[C@H](NC(=O)c4cc5cc(C(F)(F)P(=O)(O)O)ccc5s4)C(=O)N32)C1] | 635.58 | ¹H NMR (600 MHz, DMSO-d₆) δ 8.76 (d, J = 7.8 Hz, 1H), 8.29 (d, J = 13.2 Hz, 1H), 8.11 (dt, J = 10.9, 5.4 Hz, 1H), 8.06 (d, J = 8.2 Hz, 1H), 7.91 (dd, J = 13.1, 6.9 Hz, 1H), 7.60-7.55 (m, 1H), 6.20 (dd, J = 14.9, 6.5 Hz, 1H), 4.62 (dt, J = 22.3, 9.6 Hz, 2H), 4.42-4.34 (m, 2H), |
| | | | | 4.10-3.99 (m, 2H), |
| | | | | 3.10-3.02 (m, 2H), |
| | | | | 2.18 (dd, J = 13.3, 6.1 |
| | | | | Hz, 1H), 2.01-1.91 |
| | | | | (m, 2H), 1.85-1.62 |
| | | | | (m, 7H), 1.15 (t, J = |
| | | | | 7.3 Hz, 2H) |
| 810 | (difluoro(2- (((3S,6S,9aS)-3-(3- fluoro-3-(4-methyl- 4H-1,2,4-triazol-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 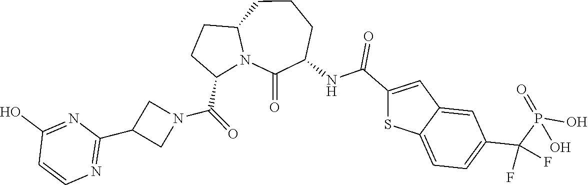 [SMILES: O=C(N[C@H]1CCC[C@H]2CC[C@@H](C(=O)N3CC(c4nccc(O)n4)C3)N2C1=O)c1cc2cc(C(F)(F)P(=O)(O)O)ccc2s1] | 640.57 | |
| 811 | ((2-(((3S,6S,9aS)-3- (3-(6-cyanopyridin- 2-yl)azetidine-1- carbonyl)-5- oxooctahydro-1H- pyrrolo[1,2- a]azepin-6- yl)carbamoyl)benzo [b]thiophen-5- yl)difluoromethyl) phosphonic acid | 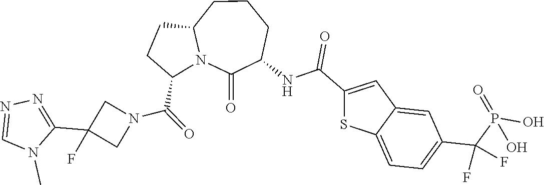 [SMILES: Cn1cnnc1C1(F)CN(C(=O)[C@@H]2CC[C@@H]3CCC[C@H](NC(=O)c4cc5cc(C(F)(F)P(=O)(O)O)ccc5s4)C(=O)N32)C1] | 643.6 | ¹H NMR (600 MHz, DMSO-d₆) δ 8.77 (t, J = 7.9 Hz, 1H), 8.28 (d, J = 19.8 Hz, 1H), 8.14-8.04 (m, 2H), 8.01 (dt, J = 12.7, 7.8 Hz, 1H), 7.92 (dd, J = 15.2, 7.6 Hz, 1H), 7.72 (dd, J = 11.0, 8.0 Hz, 1H), 7.57 (dd, J = 8.9, 4.2 Hz, 1H), 4.73 |
| | | | | (t, J = 8.7 Hz, 1H), |
| | | | | 4.64 (d, J = 10.9 Hz, |
| | | | | 1H), 4.55 (t, J = 8.5 |
| | | | | Hz, 1H), 4.44 (dt, J = |
| | | | | 9.0, 4.9 Hz, 1H), 4.32 |
| | | | | (dd, J = 8.5, 5.7 Hz, |
| | | | | 1H), 4.22 (dt, J = |
| | | | | 35.0, 9.1 Hz, 1H), |
| | | | | 4.16-4.01 (m, 2H), |
| | | | | 3.98-3.93 (m, 1H), |
| | | | | 3.11-3.06 (m, 1H), |
| | | | | 2.21-2.16 (m, 1H), |
| | | | | 2.06-1.92 (m, 2H), |
| | | | | 1.85-1.65 (m, 5H) |
| 812 | (difluoro(2- (((3S,6S,9aS)-5- oxo-3-(3-(4- (trifluoromethyl) pyrimidin-2- yl)azetidine-1- carbonyl)octahydro- 1H-pyrrolo[1,2- a]azepin-6- yl)carbamoyl)benzo [b]thiophen-5- yl)methyl) phosphonic acid | 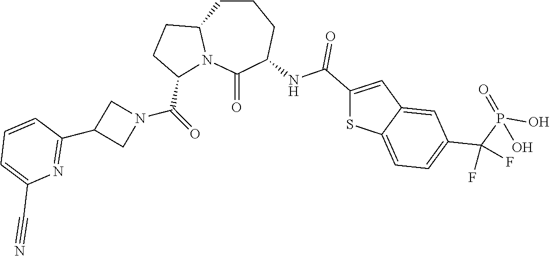 [SMILES: N#Cc1cccc(C2CN(C(=O)[C@@H]3CC[C@@H]4CCC[C@H](NC(=O)c5cc6cc(C(F)(F)P(=O)(O)O)ccc6s5)C(=O)N43)C2)n1] | 687.58 | ¹H NMR (500 MHz, DMSO-d₆) δ 9.20 (dd, J = 8.3, 5.0 Hz, 1H), 8.76 (dd, J = 26.3, 7.5 Hz, 1H), 8.28 (d, J = 24.8 Hz, 1H), 8.11 (dd, J = 8.5, 5.5 Hz, 1H), 8.05 (d, J= 14.0 Hz, 1H), 7.92 (dd, J = 16.0, 5.1 Hz, 1H), 7.61-7.54 (m, 1H), 4.80-4.57 (m, 3H), 4.46-4.43 (m, 1H), 4.30 (t, J = 9.5 Hz, 1H), 4.25 (t, J = 7.7 Hz, 1H), 2.23- |
| | | | | 2.16 (m, 1H), 2.00 |
| | | | | (ddd, J = 36.9, 19.0, |
| | | | | 10.1 Hz, 3H), 1.85- |
| | | | | 1.68 (m, 8H) |
| 813 | ((2-(((3S,6S,10aS)- 3-(2,2-dimethyl-4- phenylpyrrolidine- 1-carbonyl)-5- oxodecahydropyrrolo [1,2-a]azocin-6- yl)carbamoyl)benzo [b]thiophen-5- yl)difluoromethyl) phosphonic acid | 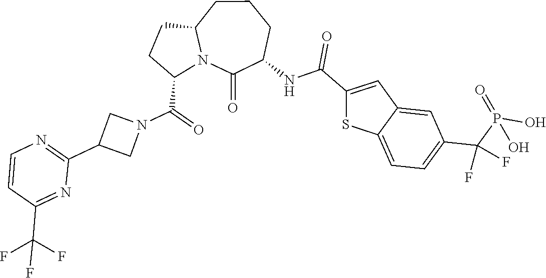 [SMILES: O=C(N[C@H]1CCC[C@H]2CC[C@@H](C(=O)N3CC(c4nccc(C(F)(F)F)n4)C3)N2C1=O)c1cc2cc(C(F)(F)P(=O)(O)O)ccc2s1] | 673.71 | ¹H NMR (400 MHz, DMSO-d₆) δ 8.90 (d, J = 7.3 Hz, 1H), 8.32 (d, J = 2.7 Hz, 1H), 8.14-8.09 (m, 1H), 8.05 (s, 1H), 7.57 (d, J = 8.6 Hz, 1H), 7.36- 7.26 (m, 4H), 7.25- 7.13 (m, 1H), 5.02- 4.91 (m, 1H), 4.60- 3.96 (m, 2H), 3.61- 3.36 (m, 2H), 2.23 (d, J = 34.6 Hz, 2H), 2.14- 2.01 (m, 3H), 1.88 (d, J = 12.0 Hz, 4H), 1.84-1.69 (m, 4H), 1.63 (s, 2H), 1.49 (d, J = 3.6 Hz, 3H), 1.40 (s, 3H) |
| 814 | ((2-(((3S,6S,10aS)- 3-((S)-2,2-dimethyl- 4-phenylpyrrolidine- 1-carbonyl)-5- oxodecahydropyrrolo [1,2-a]azocin-6- yl)carbamoyl)benzo [b]thiophen-5- yl)difluoromethyl) phosphonic acid | 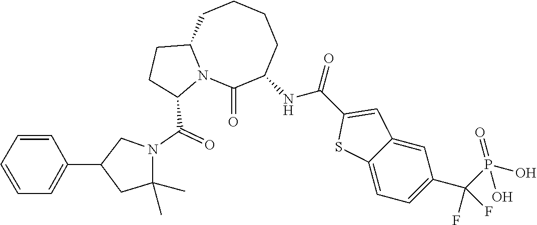 [SMILES: CC1(C)CC(c2ccccc2)CN1C(=O)[C@@H]1CC[C@@H]2CCCC[C@H](NC(=O)c3cc4cc(C(F)(F)P(=O)(O)O)ccc4s3)C(=O)N21] | 673.71 | ¹H NMR (400 MHz, DMSO-d₆) δ 8.91 (d, J = 7.3 Hz, 1H), 8.32 (s, 1H), 8.14-8.10 (m, 1H), 8.05 (s, 1H), 7.57 (d, J = 8.2 Hz, 1H), 7.34-7.27 (m, 4H), 7.21 (dd, J = 8.1, 4.7 Hz, 1H), 5.06- 4.88 (m, 1H), 4.44 (t, J = 8.3 Hz, 1H), 4.24 (t, J = 9.7 Hz, 1H), 4.00 (t, J = 8.4 Hz, 1H), 3.55 (d, J = 10.1 Hz, 1H), 3.46 (dt, J = |
| | | | | 13.0, 7.0 Hz, 1H), |
| | | | | 2.28 (s, 1H), 2.13- |
| | | | | 2.02 (m, 2H), 2.01- |
| | | | | 1.68 (m, 8H), 1.69- |
| | | | | 1.51 (m, 3H), 1.48 (s, |
| | | | | 3H), 1.40 (s, 3H) |
| 815 | ((2-(((3S,6S,10aS)- 3-((R)-2,2-dimethyl- 4-phenylpyrrolidine- 1-carbonyl)-5- oxodecahydropyrrolo [1,2-a]azocin-6- yl)carbamoyl)benzo [b]thiophen-5- yl)difluoromethyl) phosphonic acid | 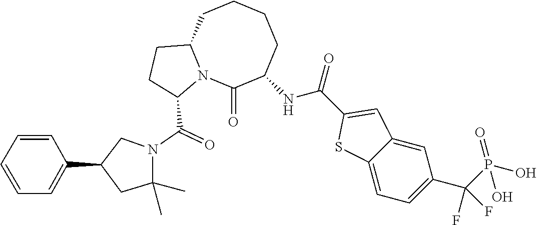 [SMILES: CC1(C)C[C@@H](c2ccccc2)CN1C(=O)[C@@H]1CC[C@@H]2CCCC[C@H](NC(=O)c3cc4cc(C(F)(F)P(=O)(O)O)ccc4s3)C(=O)N21] | 673.71 | ¹H NMR (400 MHz, DMSO-d₆) δ 8.92 (d, J = 7.3 Hz, 1H), 8.33 (s, 1H), 8.12 (d, J = 8.4 Hz, 1H), 8.05 (s, 1H), 7.57 (d, J = 8.5 Hz, 1H), 7.36-7.26 (m, 4H), 7.22 (d, J = 7.0 Hz, 1H), 5.03- 4.93 (m, 1H), 4.56 (t, J = 8.5 Hz, 1H), 4.34 (s, 1H), 4.23 (s, 1H), 3.41 (d, J = 6.8 Hz, 2H), 2.20 (d, J = 10.2 |
| | | | | Hz, 1H), 2.11-1.98 |
| | | | | (m, 3H), 1.92 (q, J = |
| | | | | 11.5 Hz, 4H), 1.75 |
| | | | | (dq, J = 26.8, 9.1, 7.2 |
| | | | | Hz, 4H), 1.64 (s, 2H), |
| | | | | 1.49 (s, 3H), 1.39 (s, |
| | | | | 3H) |
| 816 | (difluoro(2- (((3S,6S,10aS)-5- oxo-3-(3-(6-oxo- 1,6-dihydropyridin- 3-yl)azetidine-1- carbonyl)decahydro pyrrolo[1,2- a]azocin-6- yl)carbamoyl)benzo [b]thiophen-5- yl)methyl) phosphonic acid | 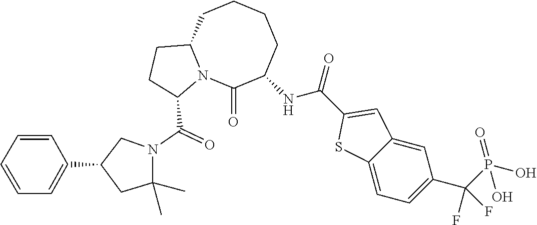 [SMILES: CC1(C)C[C@H](c2ccccc2)CN1C(=O)[C@@H]1CC[C@@H]2CCCC[C@H](NC(=O)c3cc4cc(C(F)(F)P(=O)(O)O)ccc4s3)C(=O)N21] | 648.62 | |
| 817 | ((2-(((3S,6S,9aS)-3- (3-(5- cyclopropyl- pyrimidin-2- yl)azetidine-1- carbonyl)-5- oxooctahydro-1H- pyrrolo[1,2- a]azepin-6- yl)carbamoyl)benzo [b]thiophen-5- yl)difluoromethyl) phosphonic acid | 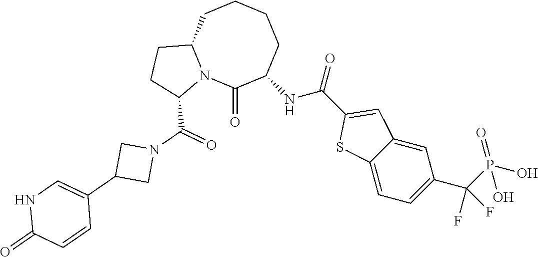 [SMILES: O=C(N[C@H]1CCCC[C@H]2CC[C@@H](C(=O)N3CC(c4ccc(=O)[nH]c4)C3)N2C1=O)c1cc2cc(C(F)(F)P(=O)(O)O)ccc2s1] | 659.65 | ¹H NMR (500 MHz, DMSO-d₆) δ 8.78 (dd, J = 13.8, 7.6 Hz, 1H), 8.55 (d, J = 13.5 Hz, 2H), 8.29 (d, J = 16.2 Hz, 1H), 8.14- 8.02 (m, 2H), 7.57 (d, J = 8.7 Hz, 1H), 4.71 (t, J = 8.4 Hz, 1H), 4.63 (q, J = 11.3, 10.7 Hz, 1H), 4.54-4.47 (m, 1H), 4.43 (q, J = 4.3 Hz, 1H), 4.36 (dd, J = 8.5, 5.0 Hz, 1H), 4.24 (d, J = 5.9 Hz, 1H), 4.15 (d, J = 11.7 |
| | | | | Hz, 1H), 4.07 (q, J = |
| | | | | 5.0, 3.8 Hz, 1H), 3.98 |
| | | | | (p, J = 7.3, 6.5 Hz, |
| | | | | 2H), 3.07 (dd, J = 7.3, |
| | | | | 4.8 Hz, 1H), 2.17 (dt, |
| | | | | J = 13.1, 6.6 Hz, 1H), |
| | | | | 2.01-1.89 (m, 2H), |
| | | | | 1.84-1.66 (m, 5H), |
| | | | | 0.99 (ddt, J = 14.9, |
| | | | | 8.5, 3.2 Hz, 2H), 0.81 |
| | | | | (ddt, J = 9.6, 6.7, 4.5 |
| | | | | Hz, 2H) |
| 818 | (difluoro(2- (((3S,6S,9aS)-3-(3- (4-methylpyridazin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 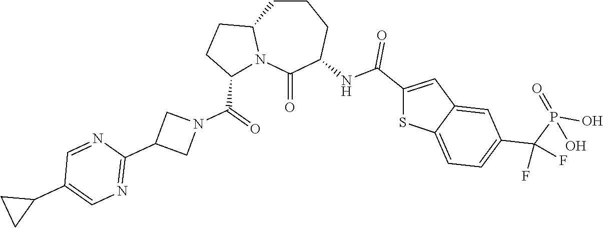 [SMILES: O=C(N[C@H]1CCC[C@H]2CC[C@@H](C(=O)N3CC(c4ncc(C5CC5)cn4)C3)N2C1=O)c1cc2cc(C(F)(F)P(=O)(O)O)ccc2s1] | 633.61 | ¹H NMR (500 MHz, DMSO-d₆) δ 9.01 (dd, J = 11.2, 5.1 Hz, 1H), 8.78 (t, J = 7.1 Hz, 1H), 8.28 (d, J = 27.0 Hz, 1H), 8.12 (dd, J = 11.8, 8.9 Hz, 1H), 8.06 (d, J = 13.9 Hz, 1H), 7.54 (dt, J = 18.8, 6.9 Hz, 2H), 4.73 (dt, J = 28.9, 7.3 Hz, 1H), 4.62 (dd, J = 16.6, 10.1 Hz, 2H), 4.46 (dd, J = 8.6, 4.1 |
| | | | | Hz, 1H), 4.35-4.32 |
| | | | | (m, 1H), 3.07 (dd, J = |
| | | | | 7.4, 4.7 Hz, 1H), 2.22 |
| | | | | (s, 3H), 2.21-2.03 |
| | | | | (m, 2H), 1.98 (d, J = |
| | | | | 44.7 Hz, 2H), 1.76 (t, |
| | | | | J = 26.5 Hz, 8H) |
| 819 | ((2-(((3S,6S,9aS)-3- (3-(4- (difluoromethyl) pyridin-3- yl)azetidine- 1-carbonyl)-5- oxooctahydro-1H- pyrrolo[1,2- a]azepin-6- yl)carbamoyl)benzo [b]thiophen-5- yl)difluoromethyl) phosphonic acid | 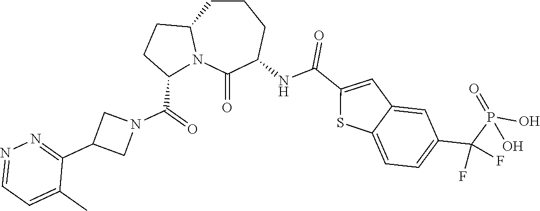 [SMILES: Cc1ccnnc1C1CN(C(=O)[C@@H]2CC[C@@H]3CCC[C@H](NC(=O)c4cc5cc(C(F)(F)P(=O)(O)O)ccc5s4)C(=O)N32)C1] | 668.6 | ¹H NMR (500 MHz, DMSO-d₆) δ 8.96 (d, J = 18.6 Hz, 1H), 8.82 (dd, J = 28.9, 7.6 Hz, 1H), 8.66 (dd, J = 14.2, 5.1 Hz, 1H), 8.29 (d, J = 8.9 Hz, 1H), 8.12 (dd, J = 9.0, 4.7 Hz, 1H), 8.08 (d, J = 5.3 Hz, 1H), 7.59- 7.50 (m, 2H), 7.20 (td, J = 54.1, 4.0 Hz, 1H), 4.80-4.46 (m, |
| | | | | 4H), 4.43 (dd, J = 8.1, |
| | | | | 4.7 Hz, 1H), 4.40- |
| | | | | 4.26 (m, 2H), 2.24- |
| | | | | 2.08 (m, 1H), 2.08- |
| | | | | 1.98 (m, 1H), 1.94 (s, |
| | | | | 1H), 1.92-1.55 (m, |
| | | | | 8H) |
| 820 | ((2-(((3S,6S,9aS)-3- (3-(6-cyano-3- methylpyridin-2- yl)azetidine-1- carbonyl)-5- oxooctahydro-1H- pyrrolo[1,2- a]azepin-6- yl)carbamoyl)benzo [b]thiophen-5- yl)difluoromethyl) phosphonic acid | 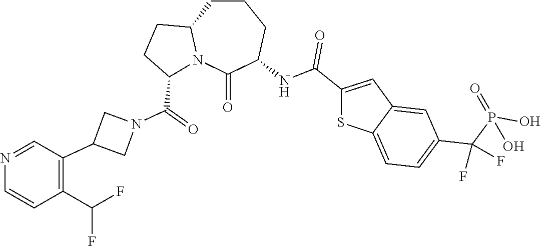 [SMILES: O=C(N[C@H]1CCC[C@H]2CC[C@@H](C(=O)N3CC(c4cnccc4C(F)F)C3)N2C1=O)c1cc2cc(C(F)(F)P(=O)(O)O)ccc2s1] | 657.63 | ¹H NMR (500 MHz, DMSO-d₆) δ 8.76 (dd, J = 14.7, 7.5 Hz, 1H), 8.27 (d, J = 28.6 Hz, 1H), 8.10 (t, J = 9.4 Hz, 1H), 8.05 (d, J = 12.7 Hz, 1H), 7.90- 7.76 (m, 2H), 7.56- (t, J = 7.7 Hz, 1H), 4.71 (t, J = 8.3 Hz, 1H), 4.64 (q, J = 9.9 Hz, 1H), 4.58-4.40 (m, 3H), 4.22 (dp, J = 19.1, 7.1, 6.5 Hz, 2H), 2.27 (d, J = 2.1 |
| | | | | Hz, 3H), 2.22-2.13 |
| | | | | (m, 1H), 2.01 (ddd, |
| | | | | J = 14.6, 7.6, 3.8 Hz, |
| | | | | 1H), 1.93 (s, 1H), |
| | | | | 1.91-1.50 (m, 7H) |
| 821 | ((2-(((3S,6S,9aS)-3- (3-(1H-indazol-1- yl)azetidine-1- carbonyl)-5- oxooctahydro-1H- pyrrolo[1,2- a]azepin-6- yl)carbamoyl)benzo [b]thiophen-5- yl)difluoromethyl) phosphonic acid | 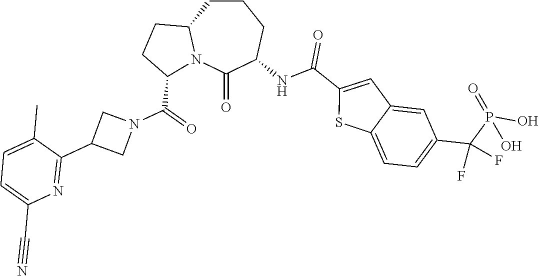 [SMILES: Cc1ccc(C#N)nc1C1CN(C(=O)[C@@H]2CC[C@@H]3CCC[C@H](NC(=O)c4cc5cc(C(F)(F)P(=O)(O)O)ccc5s4)C(=O)N32)C1] | 657.63 | ¹H NMR (500 MHz, DMSO-d₆) δ 8.82 (dd, J = 20.7, 7.6 Hz, 1H), 8.31 (d, J = 3.9 Hz, 1H), 8.20 (d, J = 22.3 Hz, 1H), 8.12 (d, J = 8.9 Hz, 1H), 8.07 (s, 1H), 7.81-7.67 (m, 2H), 7.57 (d, J = 8.6 Hz, 1H), 7.38 (q, J = 8.2 Hz, 1H), 7.14 (dt, J = 16.0, 7.5 Hz, 1H), 5.74 (hept, J = 6.1, 4.9 Hz, 1H), 4.84 (dt, J = 76.4, 7.7 Hz, 1H), 4.73-4.60 (m, 2H), 4.59-4.40 (m, |
| | | | | 3H), 2.19 (dd, J = |
| | | | | 13.1, 7.1 Hz, 1H), |
| | | | | 2.04 (tt, J = 8.1, 4.5 |
| | | | | Hz, 1H), 1.95 (s, 1H), |
| | | | | 1.90-1.77 (m, 4H), |
| | | | | 1.72 (s, 4H) |
| 822 | (difluoro(2- (((3S,6S,9aS)-3-(3- methoxy-3-((S- methylsulfonimidoy 1)methyl)azetidine- 1-carbonyl)-5- oxooctahydro-1H- pyrrolo[1,2- a]azepin-6- yl)carbamoyl)benzo [b]thiophen-5- yl)methyl) phosphonic acid | 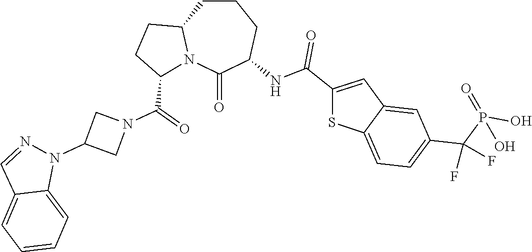 [SMILES: O=C(N[C@H]1CCC[C@H]2CC[C@@H](C(=O)N3CC(n4ncc5ccccc54)C3)N2C1=O)c1cc2cc(C(F)(F)P(=O)(O)O)ccc2s1] | 662.66 | |
| 823 | (difluoro(2- (((3S,6S,9aS)-3-(3- isopropoxy-3-(4- methyl-4H-1,2,4- triazol-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 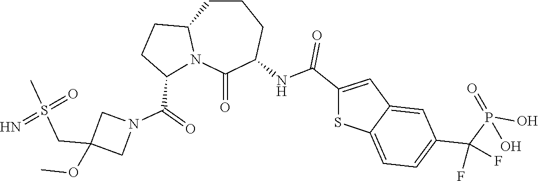 [SMILES: COC1(CS(C)(=N)=O)CN(C(=O)[C@@H]2CC[C@@H]3CCC[C@H](NC(=O)c4cc5cc(C(F)(F)P(=O)(O)O)ccc5s4)C(=O)N32)C1] | 680.66 | ¹H NMR (500 MHz, DMSO-d₆) δ 8.79 (dd, J = 32.7, 7.7 Hz, 1H), 8.53 (d, J = 16.2 Hz, 1H), 8.26 (d, J = 27.6 Hz, 1H), 8.14- 8.02 (m, 2H), 7.57 (t, J = 7.5 Hz, 1H), 4.89 (dd, J = 27.3, 9.3 Hz, 1H), 4.64 (d, J = 13.2 Hz, 1H), 4.56 (dd, J = 28.5, 10.0 Hz, 1H), 4.49-4.40 (m, 2H), 4.07 (dd, J = 10.3, 5.3 |
| | | | | Hz, 1H), 3.97 (s, 1H), |
| | | | | 3.57 (d, J = 3.6 Hz, |
| | | | | 3H), 3.40 (d, J = 5.6 |
| | | | | Hz, 1H), 3.11-3.02 |
| | | | | (m, 1H), 2.16 (dt, J = |
| | | | | 13.5, 7.2 Hz, 1H), |
| | | | | 2.07-1.92 (m, 2H), |
| | | | | 1.86-1.66 (m, 6H), |
| | | | | 0.97-0.80 (m, 6H) |
| 824 | ((2-(((3S,6S,9aS)-3- (3-(4- cyanopyrimidin-2- yl)azetidine-1- carbonyl)-5- oxooctahydro-1H- pyrrolo[1,2- a]azepin-6- yl)carbamoyl)benzo [b]thiophen-5- yl)difluoromethyl) phosphonic acid | 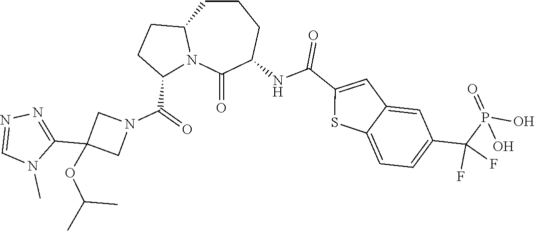 [SMILES: CC(C)OC1(c2nncn2C)CN(C(=O)[C@@H]2CC[C@@H]3CCC[C@H](NC(=O)c4cc5cc(C(F)(F)P(=O)(O)O)ccc5s4)C(=O)N32)C1] | 644.59 | ¹H NMR (500 MHz, DMSO-d₆) δ 9.14 (dd, J = 11.9, 5.0 Hz, 1H), 8.76 (dd, J = 17.9, 7.8 Hz, 1H), 8.28 (d, J = 20.3 Hz, 1H), 8.13-7.99 (m, 3H), 7.59-7.53 (m, 1H), 4.63 (h, J = 8.7, 7.7 Hz, 1H), 4.55 (q, J = 8.6, 8.1 Hz, 1H), 4.43 (ddd, J = 14.5, 8.3, 4.7 Hz, 1H), 4.21 (t, J = 3.9 Hz, 1H), |
| | | | | 4.13 (ddd, J = 13.8, |
| | | | | 9.4, 3.5 Hz, 1H), 4.04- |
| | | | | 3.91 (m, 2H), 3.09- |
| | | | | 3.00 (m, 1H), 2.18 |
| | | | | (dt, J = 14.0, 7.0 Hz, |
| | | | | 1H), 2.06-1.91 (m, |
| | | | | 2H), 1.87-1.66 (m, |
| | | | | 7H |
| 825 | (difluoro(2- (((3S,6S,10aS)-5- oxo-3- ((quinoxalin-6- ylmethyl)carbamoyl) decahydropyrrolo [1,2-a]azocin-6- yl)carbamoyl)benzo [b]thiophen-5- yl)methyl) phosphonic acid | 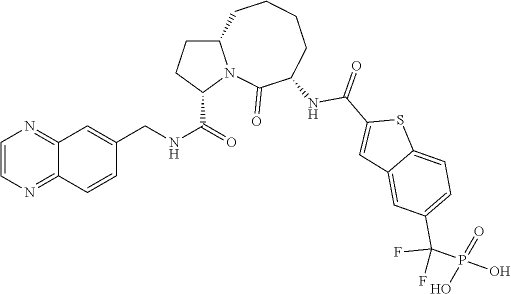 [SMILES: O=C(N[C@H]1CCCC[C@H]2CC[C@@H](C(=O)NCc3ccc4nccnc4c3)N2C1=O)c1cc2cc(C(F)(F)P(=O)(O)O)ccc2s1] | 648.62 | ¹H NMR (500 MHz, DMSO-d₆) δ 8.93- 8.87 (m, 3H), 8.71 (t, J = 6.0 Hz, 1H), 8.31 (s, 1H), 8.11 (d, J = 8.6 Hz, 1H), 8.07- 8.01 (m, 2H), 7.95 (s, 1H), 7.77 (dd, J = 8.7, 1.9 Hz, 1H), 7.56 (d, J = 8.8 Hz, 1H), 4.93 (dt, J = 12.4, 6.2 Hz, 1H), 4.59 (dd, J = 16.0, 6.0 Hz, 1H), 4.52 (dd, J = 16.0, 5.4 Hz, 1H), 4.41 (t, J = 8.6 Hz, 1H), 4.29 (dd, J = 11.9, 7.5 Hz, 1H), 3.09-3.00 (m, 1H), |
| | | | | 2.19 (dt, J = 12.4, 7.6 |
| | | | | Hz, 1H), 2.13-2.01 |
| | | | | (m, 1H), 1.94-1.78 |
| | | | | (m, 5H), 1.72 (dd, J = |
| | | | | 11.9, 6.7 Hz, 1H), |
| | | | | 1.59 (q, J = 15.7, 13.0 |
| | | | | Hz, 2H), 1.53-1.44 |
| | | | | (m, 1H) |
| 826 | (difluoro(2- (((3S,6S,9aS)-3-(3- (3-methoxypyridin- 2-yl)azetidine-1- carbonyl)-5- oxooctahydro-1H- pyrrolo[1,2- a]azepin-6- yl)carbamoyl)benzo [b]thiophen-5- yl)methyl) phosphonic acid | 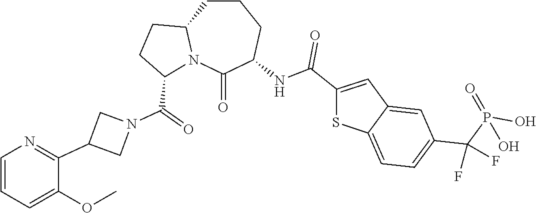 [SMILES: COc1cccnc1C1CN(C(=O)[C@@H]2CC[C@@H]3CCC[C@H](NC(=O)c4cc5cc(C(F)(F)P(=O)(O)O)ccc5s4)C(=O)N32)C1] | 676.69 | ¹H NMR (600 MHz, DMSO-d₆) δ 8.33- 8.02 (m, 5H), 7.58 (d, J = 8.6 Hz, 1H), 7.41 (dd, J = 16.0, 8.4 Hz, 1H), 7.30 (ddd, J = 18.3, 8.3, 4.7 Hz, 1H), 4.67-4.59 (m, 1H), 4.53-4.40 (m, 2H), 4.36 (dt, J = 8.0, 4.1 Hz, 1H), 4.20- 4.03 (m, 3H), 3.96 (t, J = 7.5 Hz, 1H), 3.80 |
| | | | | (s, 3H), 2.16 (dt, J = |
| | | | | 13.0, 6.5 Hz, 1H), |
| | | | | 2.06-1.91 (m, 2H), |
| | | | | 1.87-1.62 (m, 7H) |
| 827 | ((2-(((3S,6S,9aS)-3- (3-((N,S- dimethyl- sulfonimid oyl)methyl)-3- methoxyazetidine-1- carbonyl)-5- oxooctahydro-1H- pyrrolo[1,2- a]azepin-6- yl)carbamoyl)benzo [b]thiophen-5- yl)difluoromethyl) phosphonic acid | 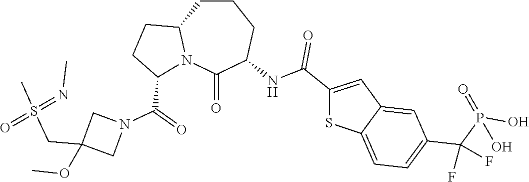 [SMILES: CN=S(C)(=O)CC1(OC)CN(C(=O)[C@@H]2CC[C@@H]3CCC[C@H](NC(=O)c4cc5cc(C(F)(F)P(=O)(O)O)ccc5s4)C(=O)N32)C1] | 645.62 | ¹H NMR (500 MHz, DMSO-d₆) δ 8.77 (dt, J = 12.6, 6.4 Hz, 1H), 8.26 (d, J = 15.3 Hz, 1H), 8.14-8.02 (m, 2H), 7.56 (d, J = 8.6 Hz, 1H), 4.68-4.56 (m, 1H), 4.44-4.30 (m, 2H), 3.95 (dt, J = 32.0, 14.2 Hz, 4H), 3.78-3.72 (m, 2H), 3.34-3.28 (m, 3H), 2.98 (d, J = 13.7 Hz, 3H), 2.60 (d, J = 31.2 |
| | | | | Hz, 3H), 2.17 (d, J = |
| | | | | 11.7 Hz, 1H), 2.02- |
| | | | | 1.90 (m, 2H), 1.85- |
| | | | | 1.64 (m, 6H) |
| 828 | (difluoro(2- (((3S,6S,10aS)-3- ((imidazo[1,2- alpyridin-7- ylmethyl) carbamoyl)-5- oxodecahydropyrrolo [1,2-a]azocin-6- yl)carbamoyl)benzo [b]thiophen-5- yl)methyl) phosphonic acid | 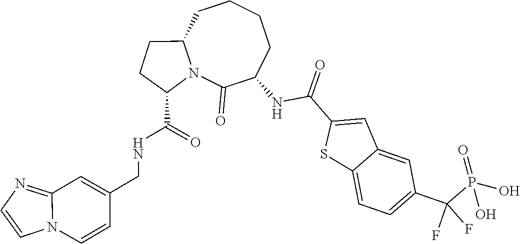 [SMILES: O=C(N[C@H]1CCCC[C@H]2CC[C@@H](C(=O)NCc3ccn4ccnc4c3)N2C1=O)c1cc2cc(C(F)(F)P(=O)(O)O)ccc2s1] | 653.62 | ¹H NMR (500 MHz, DMSO-d₆) δ 8.88- 8.77 (m, 2H), 8.30 (d, J = 9.8 Hz, 1H), 8.25 (s, 1H), 8.08 (dd, J = 16.1, 9.8 Hz, 3H), 7.73 (d, J = 14.8 Hz, 1H), 7.58 (t, J = 11.8 Hz, 2H), 7.37 (d, J = 7.1 Hz, 1H), 4.92 (s, 1H), 4.60-4.52 (m, 1H), 4.46 (s, 1H), 4.40 (t, J = 8.8 Hz, 1H), 4.30 (s, 1H), 2.21 (s, 1H), 2.09 (s, |
| | | | | 1H), 1.80 (t, J = 31.4 |
| | | | | Hz, 6H), 1.55 (d, J = |
| | | | | 40.3 Hz, 4H) |
| 829 | (difluoro(4-((E)-3- oxo-3- (((3S,6S,10aS)-5- oxo-3-(1-phenyl- 1,4,5,6- tetrahydropyrrolo[3, 4-c]pyrazole-5- carbonyl)decahydro pyrrolo[1,2- a]azocin-6- yl)amino)prop- 1-en-1- yl)phenyl)methyl) phosphonic acid | 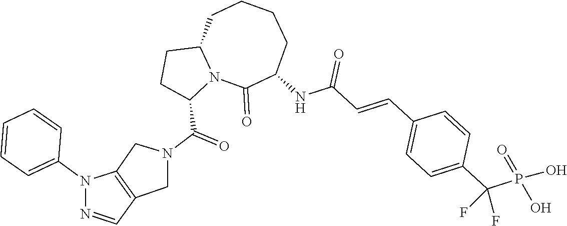 [SMILES: O=C(/C=C/c1ccc(C(F)(F)P(=O)(O)O)cc1)N[C@H]1CCCC[C@H]2CC[C@@H](C(=O)N3Cc4cnn(-c5ccccc5)c4C3)N2C1=O] | 700.65 | |
| 830 | (difluoro(2- (((3S,6S,10aS)-5- oxo-3-(1-(2-oxo- 1,2-dihydropyridin- 4-yl)-1,4,5,6- tetrahydropyrrolo[3, 4-c]pyrazole-5- carbonyl)decahydro pyrrolo[1,2- a]azocin-6- yl)carbamoyl)benzo [b]thiophen-5- yl)methyl) phosphonic acid | 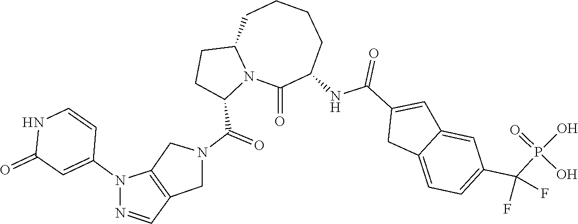 [SMILES: O=C(N[C@H]1CCCC[C@H]2CC[C@@H](C(=O)N3Cc4cnn(-c5cc[nH]c(=O)c5)c4C3)N2C1=O)C1=Cc2cc(C(F)(F)P(=O)(O)O)ccc2C1] | 648.62 | |
| 831 | ((S)-fluoro(2- (((3S,6S,9aS)-3- ((R)-6-(1-methyl-2- oxo-1,2- dihydropyridin-4- yl)-4- azaspiro[2.4] heptane-4- carbonyl)-5- oxooctahydro-1H- pyrrolo[1,2- a]azepin-6- yl)carbamoyl)benzo [b]thiophen-5- yl)methyl) phosphonic acid | 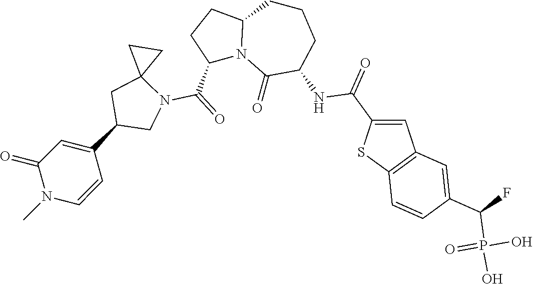 [SMILES: Cn1ccc([C@@H]2CN(C(=O)[C@@H]3CC[C@@H]4CCC[C@H](NC(=O)c5cc6cc([C@@H](F)P(=O)(O)O)ccc6s5)C(=O)N43)C3(CC3)C2)cc1=O] | 671.2 | ¹H NMR (400 MHz, DMSO-d₆) δ 8.81- 8.68 (m, 1H), 8.23 (s, 1H), 8.09-7.93 (m, 2H), 7.67-7.59 (m, 1H), 7.56-7.46 (m, 1H), 6.33 (s, 1H), 6.29-6.23 (m, 1H), 5.91-5.65 (m, 1H), 4.69-4.57 (m, 2H), 4.24-4.17 (m, 1H), 3.96-3.91 (m, 1H), 3.56-3.53 (m, 1H), 3.37 (s, 3H), 2.24- 2.13 (m, 2H), 2.07- 1.91 (m, 4H), 1.87- 1.50 (m, 9H), 0.58- 0.36 (m, 2H) |
| 832 | ((S)-(7- (((3S,6S,10aS)-3- ((6S,7R)-7-cyano-6- phenyl-4- azaspiro[2.4] heptane-4- carbonyl)-5- oxodecahydropyrrolo [1,2-a]azocin-6- yl)carbamoyl) naphthalen-2- yl)fluoromethyl) phosphonic acid | 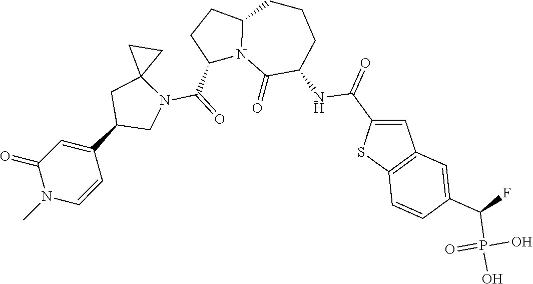 [SMILES: Cn1ccc([C@@H]2CN(C(=O)[C@@H]3CC[C@@H]4CCC[C@H](NC(=O)c5cc6cc([C@@H](F)P(=O)(O)O)ccc6s5)C(=O)N43)C3(CC3)C2)cc1=O] | 673.2 | ¹H NMR (400 MHz, DMSO-d₆) δ 8.77- 8.63 (m, 1H), 8.56 (s, 1H), 8.08-7.93 (m, 4H), 7.70-7.63 (m, 1H), 7.54-7.28 (m, 5H), 6.01-5.85 (m, 1H), 5.05-4.98 (m, 1H), 4.55-4.44 (m, 2H), 4.31-4.24 (m, 1H), 4.07-3.98 (m, 1H), 3.77-3.67 (m, 2H), 2.25-1.49 (m, 14H), 0.88-0.61 (m, 2H) |
| 833 | ((R)-(2- (((3S,6S,9aS)-3- ((6S,7R)-7-cyano-6- (pyridin-3-yl)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid | 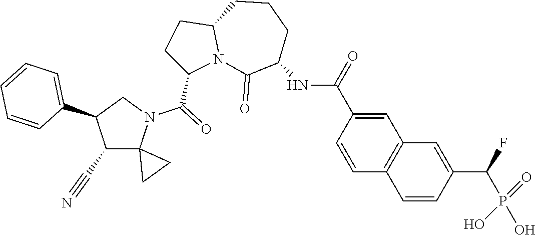 [SMILES: N#C[C@@H]1[C@@H](c2ccccc2)CN(C(=O)[C@@H]2CC[C@@H]3CCC[C@H](NC(=O)c4ccc5ccc([C@@H](F)P(=O)(O)O)cc5c4)C(=O)N32)C12CC2] | 666.3 | ¹H NMR (400 MHz, DMSO-d₆) δ 8.78 (d, J = 7.6 Hz, 1H), 8.70 (s, 1H), 8.52 (d, J = 4.4 Hz, 1H), 8.24 (s, 1H), 8.03 (d, J = 8.4 Hz, 1H), 7.99-7.86 (m, 2H), 7.53 (d, J = 8.5 Hz, 1H), 7.46- 7.33 (m, 1H), 5.82 (dd, J = 44.4, 8.0 Hz, 1H), 4.68-4.56 (m, 2H), 4.20-4.11 (m, 1H), 4.00-3.86 (m, |
| | Or | | | 4H), 2.26-1.91 (m, |
| | ((R)-(2- | SFC Pyrrolidine + core peak 1 | | 4H), 1.89-1.64 (m, |
| | (((3S,6S,9aS)-3-(( | was used for synthesis and | | 8H), 0.84-0.58 (m, |
| | 6R,7S)-7-cyano-6- | biological testing | | 2H) |
| | (pyridin-3-yl)-4- | | | |
| | azaspiro[2.4]heptane- | | | |
| | 4-carbonyl)-5- | | | |
| | oxooctahydro-1H- | | | |
| | pyrrolo[1,2- | | | |
| | a]azepin-6- | | | |
| | yl)carbamoyl)benzo | | | |
| | [b]thiophen-5- | | | |
| | yl)fluoromethyl) | | | |
| | phosphonic acid | | | |
| 834 | ((R)-fluoro(2- (((3S,6S,8R,9aR)-8- methyl-5-oxo-3- ((R)-6-(pyridin-3- yl)-4- azaspiro[2.4] heptane-4- carbonyl)octahydro- 1H-pyrrolo[1,2- a]azepin-6- yl)carbamoyl)benzo [b]thiophen-5- yl)methyl) phosphonic acid | 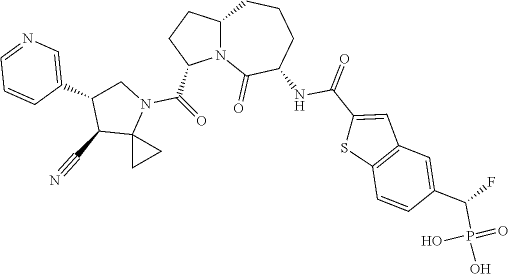 [SMILES: N#C[C@H]1[C@H](c2cccnc2)CN(C(=O)[C@@H]2CC[C@@H]3CCC[C@H](NC(=O)c4cc5cc([C@H](F)P(=O)(O)O)ccc5s4)C(=O)N32)C12CC2] | 655.0 | ¹H NMR (400 MHz, DMSO-d₆) δ 8.79 (d, J = 7.4 Hz, 1H), 8.59 (s, 1H), 8.45 (d, J = 4.3 Hz, 1H), 8.25 (s, 1H), 8.07-7.93 (m, 2H), 7.87-7.78 (m, 1H), 7.53 (d, J = 8.5 Hz, 1H), 7.41-7.33 (m, 1H), 5.81 (dd, J = 44.6, 7.4 Hz, 1H), 4.67-4.61 (m, 2H), 4.13-4.07 (m, 1H), 4.01-3.94 (m, 1H), 3.77-3.70 (m, 1H), 3.63-3.55 (m, 1H), 2.22-1.90 (m, 6H), |
| | | | | 1.83-1.40 (m, 7H), |
| | | Or | | 0.97 (d, J = 6.3 Hz, |
| | | | | 3H), 0.61-0.35 (m, |
| | | 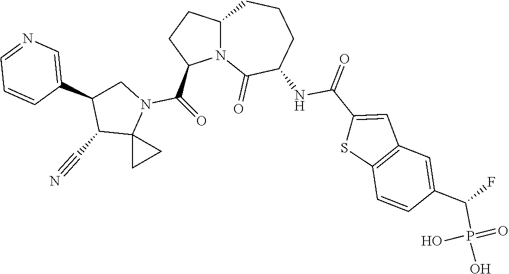 [SMILES: N#C[C@@H]1[C@@H](c2cccnc2)CN(C(=O)[C@H]2CC[C@@H]3CCC[C@H](NC(=O)c4cc5cc([C@H](F)P(=O)(O)O)ccc5s4)C(=O)N32)C12CC2] | | 2H) |
| 835 | ((R)-(2- (((3S,6S,10aS)-3- ((6S,7R)-7-cyano-6- phenyl-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 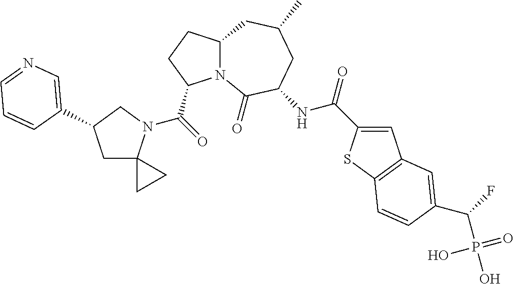 [SMILES: C[C@H]1C[C@H]2CC[C@@H](C(=O)N3C[C@@H](c4cccnc4)CC34CC4)N2C(=O)[C@@H](NC(=O)c2cc3cc([C@H](F)P(=O)(O)O)ccc3s2)C1] | 679.4 | ¹H NMR (400 MHz, DMSO-d₆) δ 8.86 (d, J = 6.9 Hz, 1H), 8.28 (s, 1H), 8.06-7.90 (m, 2H), 7.56-7.44 (m, 3H), 7.42-7.29 (m, 3H), 5.91-5.70 (m, 1H), 5.01-4.88 (m, 1H), 4.57-4.39 (m, 2H), 4.29-4.14 (m, 1H), 4.03 (d, J = 9.1 Hz, 1H), 3.79-3.63 (m, 2H), 2.30-2.14 (m, 2H), 2.08-1.95 (m, 1H), 1.94-1.63 (m, 9H), 1.60-1.45 (m, 2H), 0.87-0.74 |
| | | | | (m, 1H), 0.73-0.59 |
| | | | | (m, 1H) |
| 836 | ((S)-(2- (((3S,6S,10aS)-3- ((6R,7S)-7-cyano-6- phenyl-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 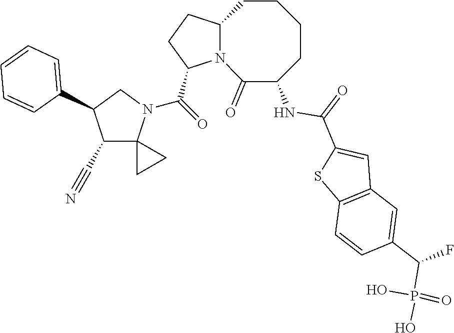 [SMILES: N#C[C@@H]1[C@@H](c2ccccc2)CN(C(=O)[C@@H]2CC[C@@H]3CCCC[C@H](NC(=O)c4cc5cc([C@H](F)P(=O)(O)O)ccc5s4)C(=O)N32)C12CC2] | 679.4 | ¹H NMR (400 MHz, DMSO-d₆) δ 8.83 (d, J = 6.7 Hz, 1H), 8.27 (s, 1H), 8.06-7.90 (m, 2H), 7.58-7.42 (m, 3H), 7.40-7.27 (m, 3H), 5.81 (dd, J = 44.4, 7.8 Hz, 1H), 5.00-4.89 (m, 1H), 4.54-4.42 (m, 1H), 4.31-4.12 (m, 2H), 3.94-3.75 (m, 3H), 2.35-2.21 (m, 1H), 2.20-2.08 (m, 1H), 2.06-1.97 (m, 1H), 1.96-1.71 (m, 8H), 1.68-1.60 (m, 1H), 1.59-1.42 (m, 2H), 0.87-0.76 (m, 1H), 0.71-0.58 (m, 1H) |
| 837 | ((S)-(2- (((3S,6S,9aS)-3- ((6S,7R)-7-cyano-6- (pyridin-3-yl)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid Or ((S)-(2- (((3S,6S,9aS)-3-(( 6R,7S)-7-cyano-6- (pyridin-3-yl)-4- azaspiro[2.4] heptane-4- | 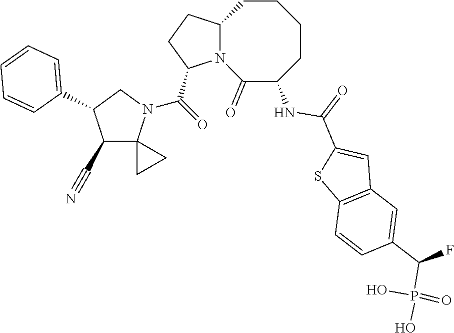 [SMILES: N#C[C@H]1[C@H](c2ccccc2)CN(C(=O)[C@@H]2CC[C@@H]3CCCC[C@H](NC(=O)c4cc5cc([C@@H](F)P(=O)(O)O)ccc5s4)C(=O)N32)C12CC2] | 666.3 | ¹H NMR (400 MHz, DMSO-d₆) δ 8.79 (d, J = 7.7 Hz, 1H), 8.72 (d, J = 1.9 Hz, 1H), 8.54 (dd, J = 4.8, 1.5 Hz, 1H), 8.23 (s, 1H), 8.02 (d, J = 8.4 Hz, 1H), 7.99-7.90 (m, 2H), 7.56-7.33 (m, 2H), 5.79 (dd, J = 44.4, 8.0 Hz, 1H), 4.70-4.56 (m, 2H), 4.36 (t, J = 8.5 Hz, 1H), 4.11-4.02 (m, 1H), 4.00-3.92 (m, 1H), 3.89-3.73 (m, 2H), 2.25-2.10 (m, 2H), 2.09-1.90 (m, 2H), 1.88-1.55 (m, 8H), 0.90-0.58 (m, 2H) |
| | carbonyl)-5- | | | |
| | oxooctahydro-1H- | SFC Pyrrolidine + core peak 2 | | |
| | pyrrolo[1,2- | was used for synthesis and | | |
| | a]azepin-6- | biological testing | | |
| | yl)carbamoyl)benzo | | | |
| | [b]thiophen-5- | | | |
| | yl)fluoromethyl) | | | |
| | phosphonic acid | | | |
| 838 | ((S)-(2- (((3S,6S,9aS)-3- ((6S,7R)-7-cyano-6- (pyridin-3-yl)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid Or ((S)-(2- (((3S,6S,9aS)-3- ((6R,7S)-7-cyano-6- (pyridin-3-yl)-4- azaspiro[2.4] | 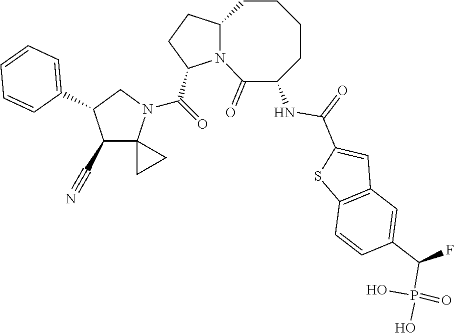 [SMILES: N#C[C@H]1[C@H](c2ccccc2)CN(C(=O)[C@@H]2CC[C@@H]3CCCC[C@H](NC(=O)c4cc5cc([C@@H](F)P(=O)(O)O)ccc5s4)C(=O)N32)C12CC2] | 666.2 | ¹H NMR (400 MHz, DMSO-d₆) δ 8.85 (s, 1H), 8.77 (d, J = 7.6 Hz, 1H), 8.67 (d, J = 4.9 Hz, 1H), 8.29- 8.22 (m, 2H), 8.04 (d, J = 8.4 Hz, 1H), 7.99 (s, 1H), 7.71-7.64 (m, 1H), 7.54 (d, J = 8.5 Hz, 1H), 5.84 (dd, J = 44.3, 8.0 Hz, 1H), 4.67-4.63 (m, 2H), 4.21-4.17 (m, 1H), 4.05-3.96 (m, 4H), 2.26-1.93 (m, 4H), 1.90-1.66 (m, 8H), 0.83-0.61 (m, 2H) |
| | heptane-4- | | | |
| | carbonyl)-5- | Or | | |
| | oxooctahydro-1H- | | | |
| | pyrrolo[1,2- a]azepin-6- yl)carbamoyl)benzo [b]thiophen-5- yl)fluoromethyl) phosphonic acid | 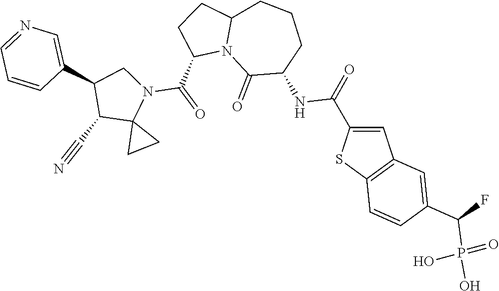 [SMILES: N#C[C@@H]1[C@@H](c2cccnc2)CN(C(=O)[C@@H]2CCC3CCC[C@H](NC(=O)c4cc5cc([C@@H](F)P(=O)(O)O)ccc5s4)C(=O)N32)C12CC2] | | |
| | | SFC Pyrrolidine + core peak 1 | | |
| | | was used for synthesis and | | |
| | | biological testing | | |
| 839 | ((R)-(2- (((3S,6S,10aS)-3- ((6S,7R)-7-cyano-6- (pyridin-3-yl)-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid Or ((R)-(2- (((3S,6S,10aS)-3- ((6R,7S)-7-cyano-6- | 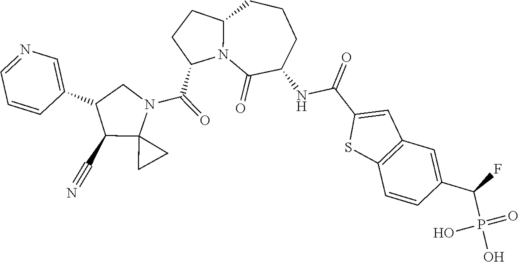 | 680.5 | ¹H NMR (400 MHz, DMSO-d₆) δ 8.92- 8.76 (m, 2H), 8.72- 8.60 (m, 1H), 8.30- 8.11 (m, 2H), 8.03 (d, J = 8.2 Hz, 1H), 7.97 (s, 1H), 7.74-7.59 (m, 1H), 7.53 (d, J = 8.5 Hz, 1H), 5.84 (dd, J = 44.4, 7.7 Hz, 1H), 4.99-4.89 (m, 1H), 4.63-4.44 (m, 2H), 4.30-4.18 (m, 1H), 4.14-4.03 (m, 1H), 3.98-3.74 (m, 2H), 2.30-2.14 (m, 2H), 2.09-1.97 (m, 1H), |
| | (pyridin-3-yl)-4- | | | 1.93-1.49 (m, 11H), |
| | azaspiro[2.4]heptane- | Or | | 0.86-0.77 (m, 1H), |
| | 4-carbonyl)-5- | | | 0.75-0.61 (m, 1H) |
| | oxodecahydropyrrolo [1,2-a]azocin-6- yl)carbamoyl)benzo [b]thiophen-5- yl)fluoromethyl) phosphonic acid | 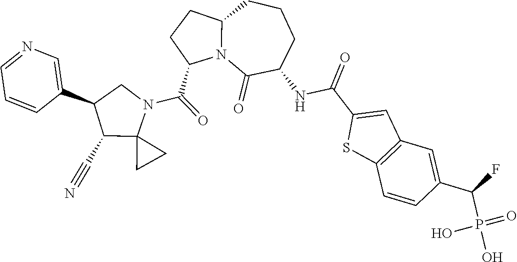 [SMILES: N#C[C@@H]1[C@@H](c2cccnc2)CN(C(=O)[C@@H]2CC[C@@H]3CCC[C@H](NC(=O)c4cc5cc([C@@H](F)P(=O)(O)O)ccc5s4)C(=O)N32)C12CC2] | | |
| | | SFC Pyrrolidine + core peak 1 | | |
| | | was used for synthesis and | | |
| | | biological testing | | |
| 840 | ((R)-(2- (((3S,6S,10aS)-3- ((6S,7R)-7-cyano-6- (pyridin-3-yl)-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid Or ((R)-(2- (((3S,6S,10aS)-3- ((6R,7S)-7-cyano-6- (pyridin-3-yl)-4- azaspiro[2.4]heptane- 4-carbonyl)-5- | 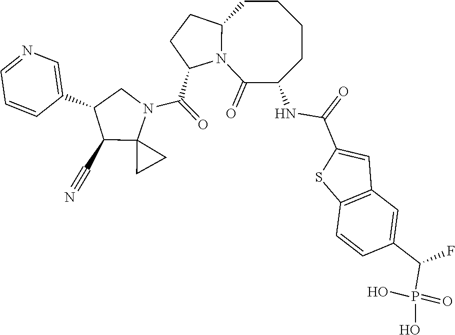 [SMILES: N#C[C@H]1[C@H](c2cccnc2)CN(C(=O)[C@@H]2CC[C@@H]3CCCC[C@H](NC(=O)c4cc5cc([C@H](F)P(=O)(O)O)ccc5s4)C(=O)N32)C12CC2] | 680.5 | ¹H NMR (400 MHz, DMSO-d₆) δ 8.92- 8.76 (m, 2H), 8.72- 8.60 (m, 1H), 8.30- 8.11 (m, 2H), 8.03 (d, J = 8.2 Hz, 1H), 7.97 (s, 1H), 7.74-7.59 (m, 1H), 7.53 (d, J = 8.5 Hz, 1H), 5.84 (dd, J = 44.4, 7.7 Hz, 1H), 4.99-4.89 (m, 1H), 4.63-4.44 (m, 2H), 4.30-4.18 (m, 1H), 4.14-4.03 (m, 1H), 3.98-3.74 (m, 2H), 2.30-2.14 (m, 2H), 2.09-1.97 (m, 1H), 1.93-1.49 (m, 11H), 0.86-0.77 (m, 1H), 0.75-0.61 (m, 1H) |
| | oxodecahydropyrrolo | | | |
| | [1,2-a]azocin-6- | Or | | |
| | yl)carbamoyl)benzo | | | |
| | [b]thiophen-5- yl)fluoromethyl) phosphonic acid | 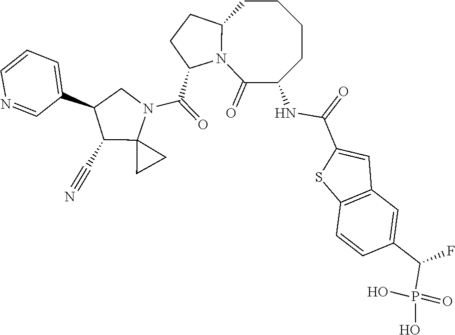 [SMILES: N#C[C@@H]1[C@@H](c2cccnc2)CN(C(=O)[C@@H]2CC[C@@H]3CCCC[C@H](NC(=O)c4cc5cc([C@H](F)P(=O)(O)O)ccc5s4)C(=O)N32)C12CC2] | | |
| | | SFC Pyrrolidine + core peak 2 | | |
| | | was used for synthesis and | | |
| | | biological testing | | |
| 841 | ((R)-(2- (((3S,6S,9aS)-3- ((6S,7R)-6-(5- chloropyridin-3-yl)- 7-cyano-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl)pho sphonic acid Or ((R)-(2- (((3S,6S,9aS)-3- ((6R,7S)-6-(5- chloropyridin-3-yl)- 7-cyano-4- | 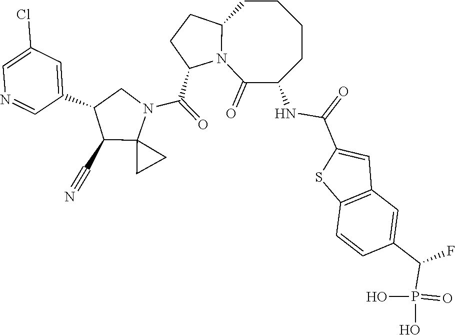 [SMILES: N#C[C@H]1[C@H](c2cncc(Cl)c2)CN(C(=O)[C@@H]2CC[C@@H]3CCCC[C@H](NC(=O)c4cc5cc([C@H](F)P(=O)(O)O)ccc5s4)C(=O)N32)C12CC2] | 700 | ¹H NMR (400 MHz, DMSO-d₆) δ 8.79 (d, J = 7.7 Hz, 1H), 8.69 (d, J = 1.4 Hz, 1H), 8.62 (d, J = 2.2 Hz, 1H), 8.25 (s, 1H), 8.20-8.14 (m, 1H), 8.06-7.96 (m, 2H), 7.53 (d, J = 8.5 Hz, 1H), 5.84 (dd, J = 44.3, 8.0 Hz, 1H), 4.66-4.59 (m, 2H), 4.39-4.31 (m, 1H), 4.13 (d, J = 10.3 Hz, 1H), 3.98-3.87 (m, 2H), 3.83-3.76 (m, 1H), 2.26-2.13 (m, 2H), 2.11-1.93 (m, 2H), 1.86-1.66 (m, 8H), 0.89-0.78 (m, 1H), 0.76-0.63 (m, |
| | azaspiro[2.4] | | | 1H) |
| | heptane-4- | Or | | |
| | carbonyl)-5- | | | |
| | oxooctahydro-1H- pyrrolo[1,2- a]azepin-6- yl)carbamoyl)benzo [b]thiophen-5- yl)fluoromethyl) phosphonic acid | 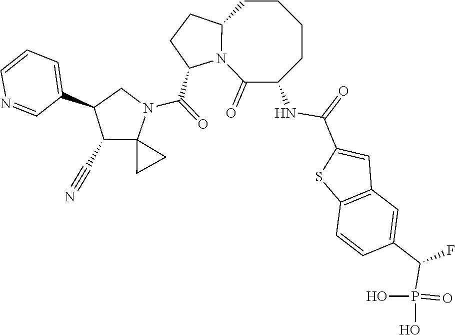 [SMILES: N#C[C@@H]1[C@@H](c2cccnc2)CN(C(=O)[C@@H]2CC[C@@H]3CCCC[C@H](NC(=O)c4cc5cc([C@H](F)P(=O)(O)O)ccc5s4)C(=O)N32)C12CC2] | | |
| | | SFC Pyrrolidine + core peak 2 | | |
| | | was used for synthesis and | | |
| | | biological testing | | |
| 842 | ((R)-(2- (((3S,6S,9aS)-3- ((6S,7R)-6-(5- chloropyridin-3-yl)- 7-cyano-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid Or ((R)-(2- (((3S,6S,9aS)-3- | 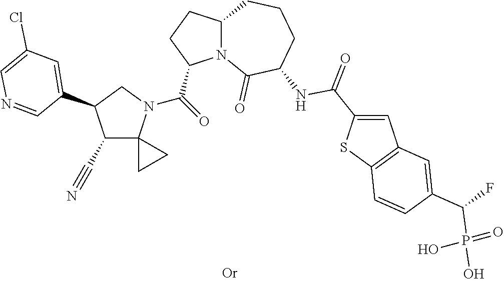 [SMILES: N#C[C@@H]1[C@@H](c2cncc(Cl)c2)CN(C(=O)[C@@H]2CC[C@@H]3CCC[C@H](NC(=O)c4cc5cc([C@H](F)P(=O)(O)O)ccc5s4)C(=O)N32)C12CC2] | 700.3 | ¹H NMR (400 MHz, DMSO) δ 8.77 (d, J = 7.0 Hz, 1H), 8.71- 8.65 (m, 1H), 8.62- 8.55 (m, 1H), 8.27- 8.16 (m, 2H), 8.04 (d, J = 8.4 Hz, 1H), 7.99 (s, 1H), 7.54 (d, J = 8.2 Hz, 1H), 5.84 (dd, J = 44.3, 7.7 Hz, 1H), 4.67-4.57 (m, 2H), 4.02-3.93 (m, 5H), 2.23-2.07 (m, 2H), 2.05-1.93 (m, 2H), 1.90-1.67 (m, 8H), 0.82-0.57 (m, 2H) |
| | ((6R,7S)-6-(5- | | | |
| | chloropyridin-3-yl)- 7-cyano-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid | 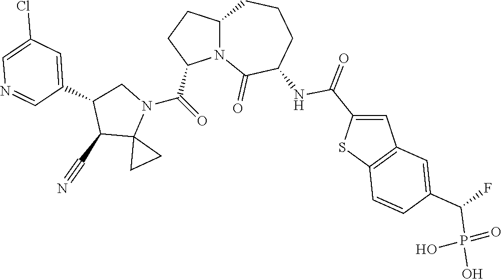 [SMILES: N#C[C@H]1[C@H](c2cncc(Cl)c2)CN(C(=O)[C@@H]2CC[C@@H]3CCC[C@H](NC(=O)c4cc5cc([C@H](F)P(=O)(O)O)ccc5s4)C(=O)N32)C12CC2] | | |
| | | SFC Pyrrolidine + core peak 1 | | |
| | | was used for synthesis and | | |
| | | biological testing | | |
| 843 | ((R)-(2- (((3S,6S,9aS)-3- ((6R,7S)-7-cyano-6- (5-methoxypyridin- 3-yl)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl)pho sphonic acid Or ((R)-(2- | 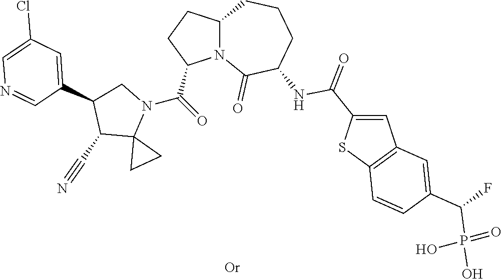 [SMILES: N#C[C@@H]1[C@@H](c2cncc(Cl)c2)CN(C(=O)[C@@H]2CC[C@@H]3CCC[C@H](NC(=O)c4cc5cc([C@H](F)P(=O)(O)O)ccc5s4)C(=O)N32)C12CC2] | 696.1 | ¹H NMR (400 MHz, DMSO-d₆) δ 8.80 (d, J = 7.7 Hz, 1H), 8.40- 8.32 (m, 2H), 8.25 (s, 1H), 8.03 (d, J = 8.5 Hz, 1H), 7.98 (s, 1H), 7.75 (s, 1H), 7.53 (d, J = 8.5 Hz, 1H), 5.84 (dd, J = 44.3, 8.0 Hz, 1H), 4.66-4.60 (m, 2H), 4.38-4.33 (m, 1H), 4.14 (d, J = 10.2 Hz, 1H), 3.99-3.94 (m, 1H), 3.91-3.83 (m, 4H), 3.78 (t, J = 9.6 Hz, 1H), 2.24- 2.15 (m, 2H), 2.08- |
| | (((3S,6S,9aS)-3- | | | 1.90 (m, 2H), 1.89- |
| | ((6S,7R)-7-cyano-6- (5-methoxypyridin- 3-y1)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid | 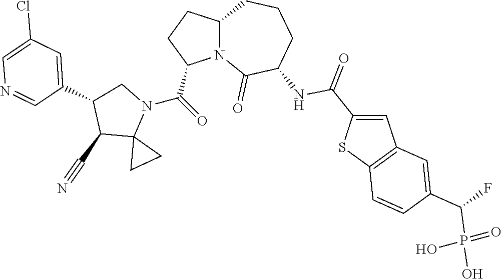 [SMILES: N#C[C@H]1[C@H](c2cncc(Cl)c2)CN(C(=O)[C@@H]2CC[C@@H]3CCC[C@H](NC(=O)c4cc5cc([C@H](F)P(=O)(O)O)ccc5s4)C(=O)N32)C12CC2] | | 1.65 (m, 8H), 0.87- 0.81 (m, 1H), 0.75- 0.68 (m, 1H) |
| | | SFC Pyrrolidine + core peak 2 | | |
| | | was used for synthesis and | | |
| | | biological testing | | |
| 844 | ((R)-(2- (((3S,6S,9aS)-3- ((6R,7S)-7-cyano-6- (5-methoxypyridin- 3-y1)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl)pho sphonic acid Or ((R)-(2- | 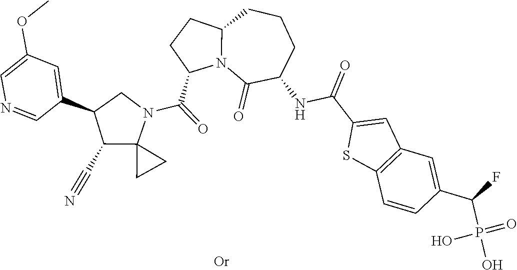 [SMILES: COc1cncc([C@H]2CN(C(=O)[C@@H]3CC[C@@H]4CCC[C@H](NC(=O)c5cc6cc([C@@H](F)P(=O)(O)O)ccc6s5)C(=O)N43)C3(CC3)[C@@H]2C#N)c1] | 696 | ¹H NMR (400 MHz, DMSO-d₆) δ 8.74 (d, J = 7.5 Hz, 1H), 8.40- 8.28 (m, 2H), 8.23 (s, 1H), 8.04 (d, J = 8.5 Hz, 1H), 7.98 (s, 1H), 7.83-7.70 (m, 1H), 7.54 (d, J = 8.5 Hz, 1H), 5.91-5.77 (m, 1H), 4.64-4.59 (m, 2H), 4.17-4.13 (m, 1H), 4.01-3.94 (m, 4H), 3.85-3.82 (m, 3H), 2.23-1.95 (m, 4H), 1.88-1.68 (m, 8H), 0.82-0.73 (m, 1H), 0.70-0.62 (m, |
| | (((3S,6S,9aS)-3-(( | | | 1H) |
| | 6S,7R)-7-cyano-6- (5-methoxypyridin- 3-yl)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid | 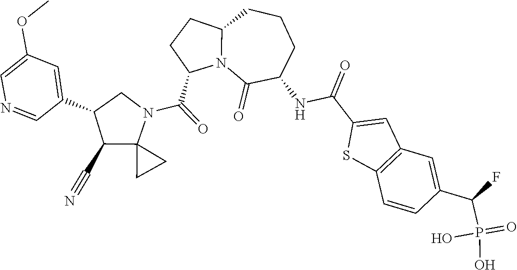 [SMILES: COc1cncc([C@@H]2CN(C(=O)[C@@H]3CC[C@@H]4CCC[C@H](NC(=O)c5cc6cc([C@@H](F)P(=O)(O)O)ccc6s5)C(=O)N43)C3(CC3)[C@H]2C#N)c1] | | |
| | | SFC Pyrrolidine + core peak 1 | | |
| | | was used for synthesis and | | |
| | | biological testing | | |
| 845 | ((R)-(2- (((3S,6S,10aS)-3- ((6S,7R)-6-(5- chloropyridin-3-yl)- 7-cyano-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid Or ((R)-(2- (((3S,6S,10aS)-3- | 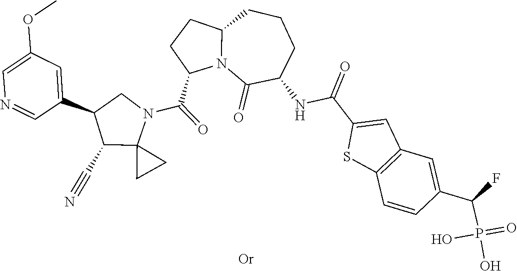 [SMILES: COc1cncc([C@H]2CN(C(=O)[C@@H]3CC[C@@H]4CCC[C@H](NC(=O)c5cc6cc([C@@H](F)P(=O)(O)O)ccc6s5)C(=O)N43)C3(CC3)[C@@H]2C#N)c1] | 714.2 | ¹H NMR (400 MHz, DMSO-d₆) δ 8.85 (d, J = 7.2 Hz, 1H), 8.67 (s, 1H), 8.61 (d, J = 2.2 Hz, 1H), 8.27 (s, 1H), 8.16 (s, 1H), 8.03 (d, J = 8.4 Hz, 1H), 7.97 (s, 1H), 7.53 (d, J = 8.5 Hz, 1H), 5.84 (dd, J = 44.3, 8.0 Hz, 1H), 4.98-4.91 (m, 1H), 4.53-4.46 (m, 2H), 4.27-4.20 (m, 1H), 4.15-4.10 (m, 1H), 3.90-3.81 (m, 2H), 2.29-2.18 (m, 2H), |
| | ((6R,7S)-6-(5- | | | 2.07-1.97 (m, 1H), |
| | chloropyridin-3-yl)- 7-cyano-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 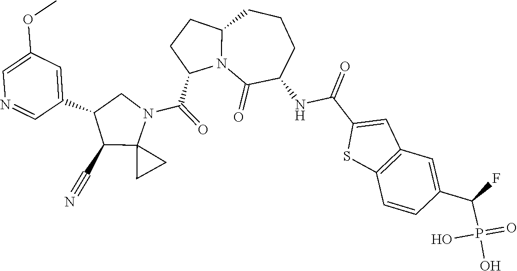 [SMILES: COc1cncc([C@@H]2CN(C(=O)[C@@H]3CC[C@@H]4CCC[C@H](NC(=O)c5cc6cc([C@@H](F)P(=O)(O)O)ccc6s5)C(=O)N43)C3(CC3)[C@H]2C#N)c1] | | 1.92-1.49 (m, 11H), 0.85-0.78 (m, 1H), 0.73-0.63 (m, 1H) |
| | | SFC Pyrrolidine + core peak 2 | | |
| | | was used for synthesis and | | |
| | | biological testing | | |
| 846 | ((R)-(2- (((3S,6S,10aS)-3- ((6S,7R)-6-(5- chloropyridin-3-yl)- 7-cyano-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid Or ((R)-(2- (((3S,6S,10aS)-3- ((6R,7S)-6-(5- chloropyridin-3-yl)- 7-cyano-4- | 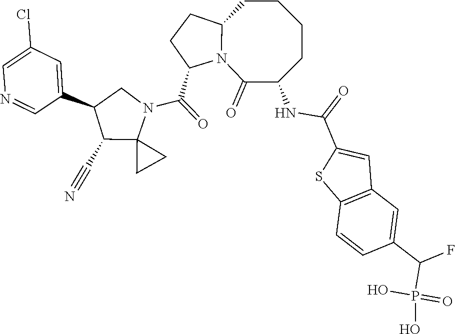 [SMILES: N#C[C@@H]1[C@@H](c2cncc(Cl)c2)CN(C(=O)[C@@H]2CC[C@@H]3CCCC[C@H](NC(=O)c4cc5cc(C(F)P(=O)(O)O)ccc5s4)C(=O)N32)C12CC2] | 714.4 | ¹H NMR (400 MHz, DMSO-d₆) δ 8.84 (d, J = 6.2 Hz, 1H), 8.72- 8.65 (m, 1H), 8.61- 8.55 (m, 1H), 8.30- 8.23 (m, 1H), 8.20- 8.12 (m, 1H), 8.03 (d, J = 8.3 Hz, 1H), 7.98 (s, 1H), 7.54 (d, J = 8.2 Hz, 1H), 5.84 (dd, J = 44.4, 7.6 Hz, 1H), 4.97-4.91 (m, 1H), 4.49-4.45 (m, 1H), 4.28-4.19 (m, 2H), 4.11-4.05 (m, 1H), 3.99-3.89 (m, 2H), 2.34-2.25 (m, 1H), 2.08-1.72 (m, 11H), 1.67-1.52 (m, 2H), 0.81-0.58 (m, 2H) |
| | azaspiro[2.4] | | | |
| | heptane-4- | Or | | |
| | carbonyl)-5- | | | |
| | oxodecahydropyrrolo [1,2-a]azocin-6- yl)carbamoyl)benzo [b]thiophen-5- yl)fluoromethyl) phosphonic acid | 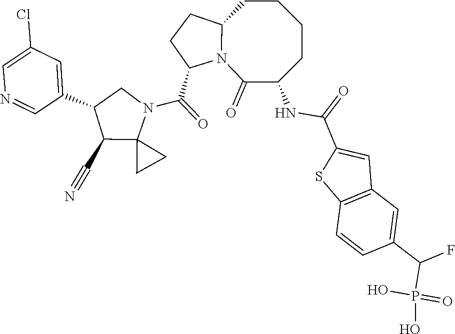 [SMILES: N#C[C@H]1[C@H](c2cncc(Cl)c2)CN(C(=O)[C@@H]2CC[C@@H]3CCCC[C@H](NC(=O)c4cc5cc(C(F)P(=O)(O)O)ccc5s4)C(=O)N32)C12CC2] | | |
| | | SFC Pyrrolidine + core peak 1 | | |
| | | was used for synthesis and | | |
| | | biological testing | | |
| 847 | ((R)-(2- (((3S,6S,9aS)-3- ((6R,7S)-7-cyano-6- (5-methoxypyridin- 3-y1)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid Or ((R)-(2- (((3S,6S,9aS)-3- ((6S,7R)-7-cyano-6- (5-methoxypyridin- 3-yl)-4- azaspiro[2.4] | 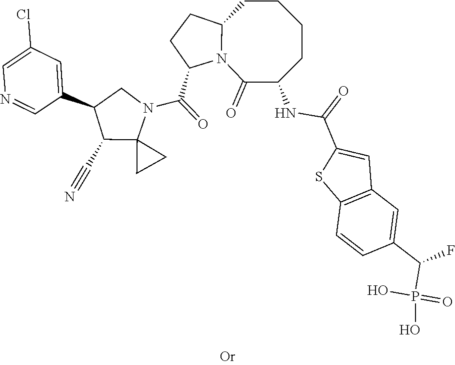 [SMILES: N#C[C@@H]1[C@@H](c2cncc(Cl)c2)CN(C(=O)[C@@H]2CC[C@@H]3CCCC[C@H](NC(=O)c4cc5cc([C@H](F)P(=O)(O)O)ccc5s4)C(=O)N32)C12CC2] | 710 | ¹H NMR (400 MHz, DMSO-d₆) δ 8.91- 8.79 (m, 1H), 8.33- 8.17 (m, 3H), 8.05- 7.91 (m, 2H), 7.60- 7.47 (m, 2H), 5.87- 5.66 (m, 1H), 5.00- 4.89 (m, 1H), 4.55- 4.44 (m, 2H), 4.26- 4.19 (m, 1H), 4.15- 4.08 (m, 1H), 3.83 (s, 3H), 3.81-3.77 (m, 2H), 2.29-2.17 (m, 2H), 2.13-1.91 (m, 2H), 1.90-1.44 (m, 10H), 0.88-0.61 (m, 2H) |
| | heptane-4- | | | |
| | carbonyl)-5- oxooctahydro-1H- pyrrolo[1,2- a]azepin-6- yl)carbamoyl)benzo [b]thiophen-5- yl)fluoromethyl) phosphonic acid | 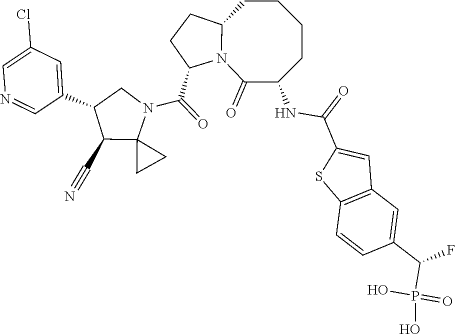 [SMILES: N#C[C@H]1[C@H](c2cncc(Cl)c2)CN(C(=O)[C@@H]2CC[C@@H]3CCCC[C@H](NC(=O)c4cc5cc([C@H](F)P(=O)(O)O)ccc5s4)C(=O)N32)C12CC2] | | |
| | | SFC Pyrrolidine + core peak 2 | | |
| | | was used for synthesis and | | |
| | | biological testing | | |
| 848 | ((R)-(2- (((3S,6S,9aS)-3- ((6R,7S)-7-cyano-6- (5-methoxypyridin- 3-yl)-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid Or ((R)-(2- (((3S,6S,9aS)-3- ((6S,7R)-7-cyano-6- (5-methoxypyridin- 3-yl)-4- azaspiro[2.4] | 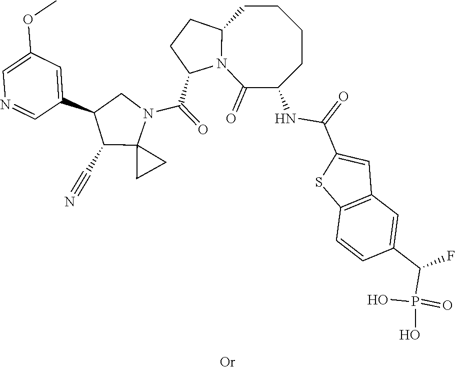 [SMILES: COc1cncc([C@H]2CN(C(=O)[C@@H]3CC[C@@H]4CCCC[C@H](NC(=O)c5cc6cc([C@H](F)P(=O)(O)O)ccc6s5)C(=O)N43)C3(CC3)[C@@H]2C#N)c1] | 710 | ¹H NMR (400 MHz, DMSO-d₆) δ 8.81 (d, J = 7.0 Hz, 1H), 8.31- 8.23 (m, 2H), 8.21 (d, J = 2.7 Hz, 1H), 8.05- 8.00 (m, 1H), 7.99- 7.96 (m, 1H), 7.58- 7.50 (m, 2H), 5.90- 5.73 (m, 1H), 4.96- 4.88 (m, 1H), 4.54- 4.48 (m, 1H), 4.28- 4.23 (m, 1H), 4.21- 4.16 (m, 1H), 4.07- 4.02 (m, 1H), 3.90- 3.86 (m, 2H), 3.81 (s, 3H), 2.36-2.23 (m, 1H), 2.07-1.97 (m, 2H), 1.96-1.50 (m, 11H), 0.79-0.71 (m, 1H), 0.67-0.58 (m, 1H |
| | heptane-4- | | | |
| | carbonyl)-5- oxooctahydro-1H- pyrrolo[1,2- a]azepin-6- yl)carbamoyl)benzo [b]thiophen-5- yl)fluoromethyl) phosphonic acid | 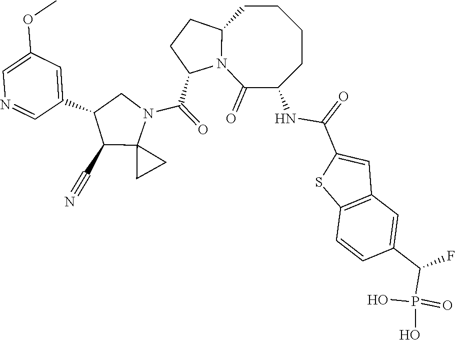 [SMILES: COc1cncc([C@@H]2CN(C(=O)[C@@H]3CC[C@@H]4CCCC[C@H](NC(=O)c5cc6cc([C@H](F)P(=O)(O)O)ccc6s5)C(=O)N43)C3(CC3)[C@H]2C#N)c1] | | |
| | | SFC Pyrrolidine + core peak 1 | | |
| | | was used for synthesis and | | |
| | | biological testing | | |
| 849 | ((R)-(2- (((3S,6S,8R,9aR)-3- ((6S,7R)-7-cyano-6- phenyl-4- azaspiro[2.4] heptane-4- carbonyl)-8- methyl-5- oxooctahydro-1H- pyrrolo[1,2- a]azepin-6- yl)carbamoyl)benzo [b]thiophen-5- yl)fluoromethyl) phosphonic acid | 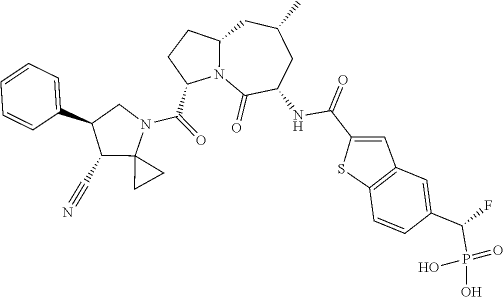 [SMILES: C[C@H]1C[C@H]2CC[C@@H](C(=O)N3C[C@H](c4ccccc4)[C@@H](C#N)C34CC4)N2C(=O)[C@@H](NC(=O)c2cc3cc([C@H](F)P(=O)(O)O)ccc3s2)C1] | 679.3 | ¹H NMR (400 MHz, DMSO-d₆) δ 8.78 (d, J = 7.1 Hz, 1H), 8.21 (s, 1H), 7.98-7.86 (m, 2H), 7.51-7.29 (m, 6H), 5.49 (dd, J = 45.4, 8.0 Hz, 1H), 4.73-4.58 (m, 2H), 4.39-4.28 (m, 1H), (m, 2H), 4.05-3.91 (m, 2H), 3.79-3.65 (m, 2H), 2.29-2.12 (m, 2H), 2.11-1.89 (m, 2H), 1.80 (d, J = 12.5 Hz, 2H), 1.68 (d, J = 10.4 Hz, 3H), 1.58-1.35 (m, 2H), 0.96 (d, J = |
| | | | | 5.2 Hz, 3H), 0.88- |
| | | | | 0.52 (m, 2H) |
| 850 | ((2-(((3S,6S,9aS)-5- oxo-3-(3-(4-(2- oxopyrrolidin-1- yl)pyridin-3- yl)azetidine-1- carbonyl)octahydro- 1H-pyrrolo[1,2- a]azepin-6- yl)carbamoyl)benzo [b]thiophen-5- yl)methyl) phosphonic acid | 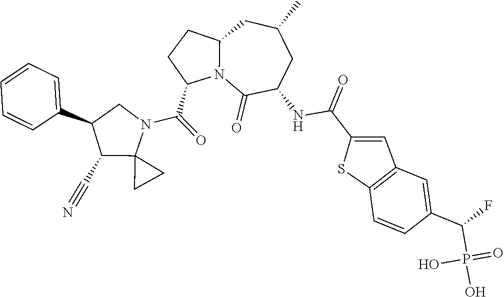 [SMILES: C[C@H]1C[C@H]2CC[C@@H](C(=O)N3C[C@H](c4ccccc4)[C@@H](C#N)C34CC4)N2C(=O)[C@@H](NC(=O)c2cc3cc([C@H](F)P(=O)(O)O)ccc3s2)C1] | 666.3 | ¹H NMR (400 MHz, CD₃OD) δ 8.94-8.88 (m, 1H), 8.63-8.41 (m, 1H), 8.04-7.94 (m, 1H), 7.85-7.76 (m, 2H), 7.69-7.60 (m, 1H), 7.42-7.39 (m, 1H), 4.89-4.74 (m, 1H), 4.74-4.59 (m, 1H), 4.59-4.45 (m, 2H), 4.41-4.29 (m, 1H), 4.19-4.08 (m, 2H), 4.06-4.01 (m, 1H), 4.01-3.91 (m, 2H), 3.30-3.19 (m, 2H), 2.68-2.57 (m, 2H), 2.38-2.24 (m, 3H), 2.22-2.08 |
| | | | | (m, 2H), 2.07-1.99 |
| | | | | (m, 2H), 1.99-1.93 |
| | | | | (m, 2H), 1.92-1.90 |
| | | | | (m, 1H), 1.87-1.84 |
| | | | | (m, 2H) |
| 851 | ((S)-fluoro(2- (((3S,6S,9aS)-3-(3- (4-(3-methyl-2- oxoimidazolidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 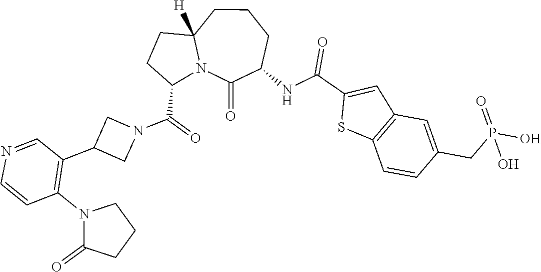 [SMILES: O=C(N[C@H]1CCC[C@H]2CC[C@@H](C(=O)N3CC(c4cnccc4N4CCCC4=O)C3)N2C1=O)c1cc2cc(CP(=O)(O)O)ccc2s1] | 699.3 | ¹H NMR (400 MHz, CD₃OD) δ 8.80-8.68 (m, 1 H) 8.33-8.15 (m, 1 H) 7.96 ( s, 1 H) 7.93 ( d, J = 11.2 Hz, 1 H) 7.84 ( d, J = 7.2 Hz, 1 H) 7.68-7.53 (m, 1 H) 7.52-7.51 (m, 1 H) 5.87-5.73 (m, 1 H) 4.78 ( d, J = 10.8 Hz, 1 H) 4.67 ( t, J = 8.4 Hz, 1 H) 4.60- 4.45 (m, 2 H) 4.39- 4.22 (m, 2 H) 4.10 ( d, J = 7.6 Hz, 1 H) 4.02- 4.01 (m, 1 H) 4.00- 3.86 (m, 2 H) 3.70- |
| | | | | 3.60 (m, 2 H) 2.94- |
| | | | | 2.89 (m, 3 H) 2.36- |
| | | | | 2.16 ( s, 1 H) 2.11- |
| | | | | 2.09 (m, 2 H) 2.08- |
| | | | | 2.00 (m, 2 H) 1.93- |
| | | | | 1.91 ( s, 2 H) 1.90- |
| | | | | 1.85 (m, 3 H) |
| 852 | ((S)-(2- (((3S,6S,9aS)-3-(3- (4-(3- (difluoromethoxy) azetidin-1-yl)pyridin- 3-yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 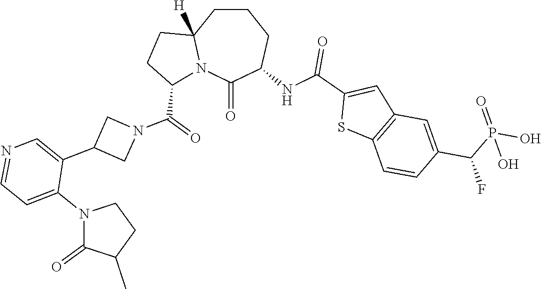 [SMILES: CC1CCN(c2ccncc2C2CN(C(=O)[C@@H]3CC[C@@H]4CCC[C@H](NC(=O)c5cc6cc([C@@H](F)P(=O)(O)O)ccc6s5)C(=O)N43)C2)C1=O] | 722.6 | ¹H NMR (400 MHz, CD₃OD) δ 8.32-8.13 (m, 1 H) 8.03 (s, 1 H) 7.98 (s, 1 H) 7.97- 7.89 (m, 1 H) 7.88- 7.71 (m, 1 H) 7.61- 7.54 (m, 1 H) 6.76- 6.33 (m, 2 H) 5.87- 5.71 (m, 1 H) 5.22- 5.11 (m, 1 H) 4.81- 4.72 (m, 3 H) 4.67- 4.52 (m, 3 H) 4.51- 4.29 (m, 3 H) 4.19- 4.08 (m, 3 H) 2.36- 2.12 (m, 2 H) 2.10- 1.95 (m, 4 H) 1.94- 1.93 (m, 4 H) |
| 853 | ((S)-(2- (((3S,6S,9aS)-3-(3- (4-(1H-imidazol-1- yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 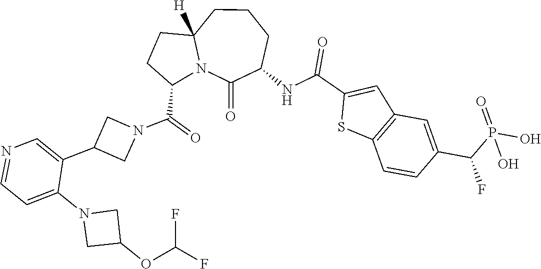 [SMILES: O=C(N[C@H]1CCC[C@H]2CC[C@@H](C(=O)N3CC(c4cnccc4N4CC(OC(F)F)C4)C3)N2C1=O)c1cc2cc([C@@H](F)P(=O)(O)O)ccc2s1] | 667.3 | ¹H NMR (400 MHz, CD₃OD, 300 K) 9.16- 9.10 (m, 1H), 9.00 (s, 1H), 8.77 (d, J = 5.2 Hz, 1H), 8.71 (s, 1H), 8.04-7.95 (m, 2H), 7.93 ( d, J = 3.2 Hz, 1H), 7.92-7.83 (m, 1H), 7.75-7.73 (m, 1H), 7.61-7.59 (m, 1H), 7.53 ( d, J = 5.2 Hz, 1H), 5.87-5.79 (m, 1H), 4.79-4.76 (m, 1H), 4.64 (d, J = 6.4 Hz, 1H), 4.55 (s, 1H), 4.50 (d, J = 2.8 Hz, 1H), 4.38-4.33 |
| | | | | (m, 1H), 4.26-4.24 |
| | | | | (m, 1H), 4.11-4.09 |
| | | | | (m, 1H), 4.04-3.99 |
| | | | | (m, 1H), 2.32-2.31 |
| | | | | (m, 1H), 2.12-2.09 |
| | | | | (m, 2H), 2.01-2.00 |
| | | | | (m, 2H), 1.99-1.98 |
| | | | | (m, 2H), 1.93-1.92 |
| | | | | (m, 1H), 1.84-1.83 |
| | | | | (m, 2H) |
| 854 | ((S)-(2- (((3S,6S,9aS)-3-(3- (4-(1H-pyrazol-1- yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 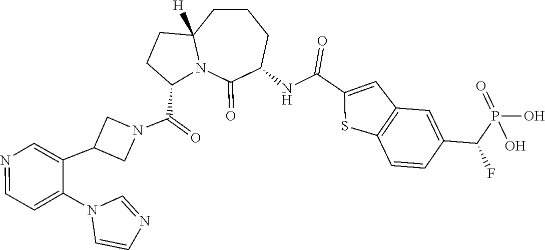 [SMILES: O=C(N[C@H]1CCC[C@H]2CC[C@@H](C(=O)N3CC(c4cnccc4-n4ccnc4)C3)N2C1=O)c1cc2cc([C@@H](F)P(=O)(O)O)ccc2s1] | 667.2 | ¹H NMR (400 MHz, CD₃OD, 300 K) δ 9.29 (d, J = 2.0 Hz, 1H), 8.87 (d, J = 6.8 Hz, 1H), 8.81 (d, J = 5.6 Hz, 1H), 8.74- 8.58 (m, 1H), 8.10 (d, J = 6.0 Hz, 1H), 8.05 ( s, 1H), 8.00-7.94 (m, 2H), 7.62-7.60 (m, 1H), 7.24-7.20 (m, 1H), 5.88-5.74 (m, 1H), 5.32-5.01 ( m, 1H), 4.79-4.76 (m, 1H), 4.73-4.65 |
| | | | | (m, 1H), 4.49-4.43 |
| | | | | (m, 1H), 4.13-4.04 |
| | | | | (m, 1H), 3.80-3.68 |
| | | | | (m, 1H), 3.56-3.52 |
| | | | | (m, 1H), 3.30-3.15 |
| | | | | (m, 1H), 2.32-2.28 |
| | | | | (m, 1H), 2.17-2.10 |
| | | | | (m, 2H), 2.08-2.05 ( |
| | | | | m, 1H), 1.99-1.94 |
| | | | | (m, 2H), 1.92-1.86 |
| | | | | (m, 2H), 1.85-1.81 |
| | | | | (m, 2H) |
| 855 | ((S)-fluoro(2- (((3S,6S,9aS)-3-(3- (4-((2- methoxyethyl) (methyl)amino) pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 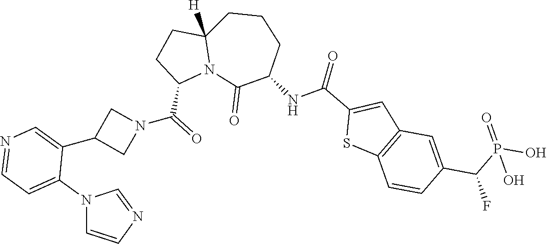 [SMILES: O=C(N[C@H]1CCC[C@H]2CC[C@@H](C(=O)N3CC(c4cnccc4-n4ccnc4)C3)N2C1=O)c1cc2cc([C@@H](F)P(=O)(O)O)ccc2s1] | 688.3 | ¹H NMR (400 MHz, CD₃OD) δ 8.24-8.19 (m, 1H), 8.12-8.01 (m, 1H), 8.01-7.94 (m, 1H), 7.94-7.86 (m, 1H), 7.86-7.80 (m, 1H), 7.64-7.52 (m, 1H), 7.19-7.01 (m, 1H), 5.86-5.70 (m, 1H), 4.77 ( d, J = 10.4 Hz, 1H), 4.69 ( t, J = 8.8 Hz, 1H), 4.60- 4.47 (m, 2H), 4.43- 4.21 (m, 2H), 4.20- 4.11 (m, 1H), 4.06- |
| | | | | 3.98 (m, 1H), 3.66- |
| | | | | 3.57 (m, 4H), 3.29- |
| | | | | 3.24 (m, 3H), 3.15- |
| | | | | 3.08 (m, 3H), 2.39- |
| | | | | 2.27 (m, 1H), 2.24- |
| | | | | 2.09 (m, 2H), 2.09- |
| | | | | 1.99 (m, 2H), 1.99- |
| | | | | 1.96 (m, 2H), 1.94- |
| | | | | 1.92 ( s, 3H) |
| 856 | ((S)-fluoro(2- (((3S,6S,9aS)-3-(3- (4-(3- (methoxymethyl) azetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 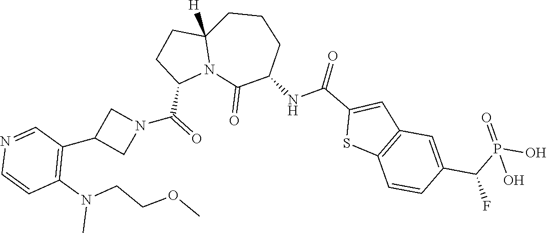 [SMILES: COCCN(C)c1ccncc1C1CN(C(=O)[C@@H]2CC[C@@H]3CCC[C@H](NC(=O)c4cc5cc([C@@H](F)P(=O)(O)O)ccc5s4)C(=O)N32)C1] | 700.3 | ¹H NMR (400 MHz, CD₃OD) δ 8.20-8.08 (m, 1 H) 8.04-7.98 (m, 2 H) 7.91-7.68 (m, 2 H) 7.61-7.57 (m, 1 H) 6.41-6.31 (m, 1 H) 5.86-5.74 (m, 1 H) 4.89-4.76 (m, 2 H) 4.56-4.54 (m, 3 H) 4.32 (t, J = 8.4 Hz, 1 H) 4.12- 4.07 (m, 5 H) 3.62- 3.59 (m, 2 H) 3.42- 3.35 (m, 3 H) 3.11- 2.99 (m, 1 H) 2.36- |
| | | | | 2.21(m, 1 H) 2.20- |
| | | | | 2.09 (m, 2 H) 2.02- |
| | | | | 1.96 (m, 4 H) 1.94- |
| | | | | 1.85 (m, 4 H) |
| 857 | ((R)-fluoro(2- (((3S,6S,9aS)-3-(3-(4- morpholino- pyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 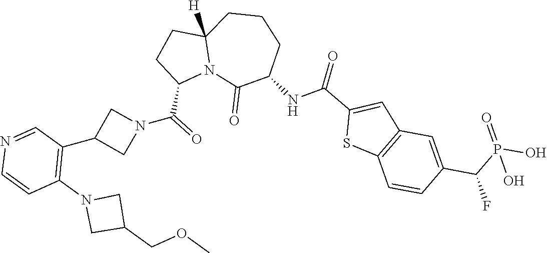 [SMILES: COCC1CN(c2ccncc2C2CN(C(=O)[C@@H]3CC[C@@H]4CCC[C@H](NC(=O)c5cc6cc([C@@H](F)P(=O)(O)O)ccc6s5)C(=O)N43)C2)C1] | 687.0 | ¹H NMR (400 MHz, CD₃OD) δ 8.53-8.22 (m, 1H), δ.17 (d, J = 19.2 Hz, 1H), 8.07- 7.96 (m, 1H), 7.85 ( s, 1H), 7.82 (d, J = 12.0 Hz, 1H), 7.59-7.52 (m, 1H), 5.86-5.65 (m, 1H), 4.74 ( d, J = 10.8 Hz, 1H), 4.67- 4.59 (m, 1H), 4.55- 4.43 (m, 2H), 4.39- 4.23 (m, 1H), 4.15- 3.97 (m, 3H), 3.83- 3.67 (m, 8H), 2.37- |
| | | | | 2.24 (m, 1H), 2.19- |
| | | | | 2.06 (m, 2H), 2.04- |
| | | | | 1.95 (m, 2H), 1.94- |
| | | | | 1.86 (m, 2H), 1.86- |
| | | | | 1.71 (m, 3H) |
| 858 | ((R)-fluoro(2- (((3S,6S,9aS)-3-(3- (4-(3- methoxyazetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 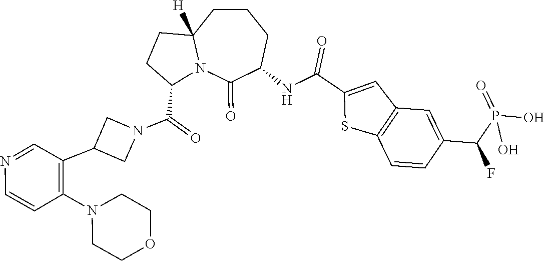 [SMILES: O=C(N[C@H]1CCC[C@H]2CC[C@@H](C(=O)N3CC(c4cnccc4N4CCOCC4)C3)N2C1=O)c1cc2cc([C@H](F)P(=O)(O)O)ccc2s1] | 686.3 | ¹H NMR (400 MHz, CD₃OD) δ 8.22-8.16 (m, 1H), 8.04-7.99 (m, 2H), 7.92-7.84 (m, 2H), 7.58 (d, J = 8.4 Hz, 1H), 6.45 (d, J = 7.2 Hz, 1H), 5.88- 5.72 (m, 1H), 4.87- 4.80 (m, 1H), 4.79- 4.74 (m, 1H), 4.65- 4.60 (m, 1H), 4.60- 4.51 (m, 3H), 4.48- 4.38 (m, 1H), 4.37- 4.32 (m, 1H), 4.31- 4.21 (m, 2H), 4.10 (t, J = 7.6 Hz, 3H), 3.36 |
| | | | | (d, J = 18.4 Hz, 3H), |
| | | | | 2.37-2.29 (m, 1H), |
| | | | | 2.19-2.09 (m, 2H), |
| | | | | 2.07-1.85 (m, 7H) |
| 859 | ((S)-fluoro(2- (((3S,6S,9aS)-3-(3- (4-(3- methoxyazetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 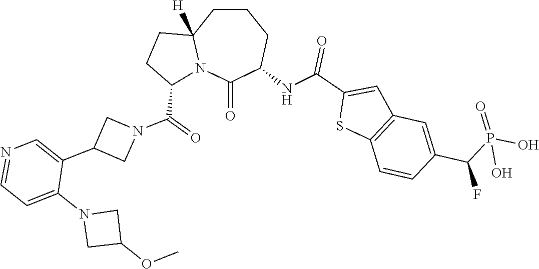 [SMILES: COC1CN(c2ccncc2C2CN(C(=O)[C@@H]3CC[C@@H]4CCC[C@H](NC(=O)c5cc6cc([C@H](F)P(=O)(O)O)ccc6s5)C(=O)N43)C2)C1] | 686.2 | ¹H NMR (400 MHz, CD₃OD) δ 8.21-8.14 (m, 1H), 8.00-7.92 (m, 2H), 7.91-7.88 (m, 2H), 7.61-7.59 (m, 1H), 6.44 (d, J = 6.8 Hz, 1H), 5.89- 5.72 (m, 1H), 4.87- 4.83 (m, 1H), 4.75 (d, J = 9.6 Hz, 1H), 4.66- 4.60 (m, 1H), 4.59- 4.54 (m, 2H), 4.52 (s, 1H), 4.42-4.35 (m, 1H), 4.35-4.17 (m, 3H), 4.16-4.04 (m, 3H), 3.36 (d, J = 19.2 Hz, 3H), 2.37-2.28 |
| | | | | (m, 1H), 2.18-2.08 |
| | | | | (m, 2H), 2.07-1.83 |
| | | | | (m, 7H) |
| 860 | ((2-(((3S,6S,9aS)-3- (3-(4-((R)-3- methylmorpholino) pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 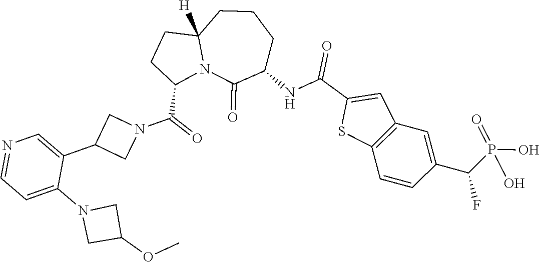 [SMILES: COC1CN(c2ccncc2C2CN(C(=O)[C@@H]3CC[C@@H]4CCC[C@H](NC(=O)c5cc6cc([C@@H](F)P(=O)(O)O)ccc6s5)C(=O)N43)C2)C1] | 682.2 | ¹H NMR (400 MHz, CD₃OD) δ 8.31 (s, 1H), 8.01 (s, 1H), 7.93 (s, 1H), 7.86- 7.77 (m, 1H), 7.74- 7.68 (m, 1H), 7.44- 7.34 (m, 1H), 7.16 (d, J = 7.2 Hz, 1H), 4.80- 4.67 (m, 2H), 4.60- 4.48 (m, 2H), 4.44- 4.29 (m, 1H), 4.22- 4.01 (m, 3H), 4.00- 3.92 (m, 1H), 3.85- 3.75 (m, 1H), 3.75- 3.63 (m, 3H), 3.62- 3.53 (m, 1H), 3.28- |
| | | | | 3.11 (m, 3H), 2.40- |
| | | | | 2.24 (m, 1H), 2.10 (s, |
| | | | | 2H), 2.06-1.96 (m, |
| | | | | 2H), 1.96-1.88 (m, |
| | | | | 2H), 1.83 (s, 3H), |
| | | | | 1.27-1.20 (m, 3H) |
| 861 | ((S)-fluoro(2- (((3S,6S,9aS)- 3-(3-(4- morpholino- pyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 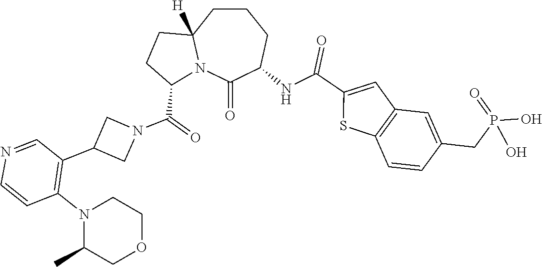 [SMILES: C[C@@H]1COCCN1c1ccncc1C1CN(C(=O)[C@@H]2CC[C@@H]3CCC[C@H](NC(=O)c4cc5cc(CP(=O)(O)O)ccc5s4)C(=O)N32)C1] | 687.2 | ¹H NMR (400 MHz, CD₃OD) δ 8.58-8.49 (m, 1H), 8.47-8.37 (m, 1H), 8.08-7.98 (m, 2H), 7.86 (d, J = 8.4, 12.5 Hz, 1H), 7.62 ( t, J = 8.0 Hz, 1H), 5.73-5.56 (m, 1H), 4.77-4.65 (m, 2H), 4.65-4.46 (m, 2H), 4.44-4.32 (m, 1H), 4.20-4.02 (m, 3H), 3.76 (t, J = 4.4 Hz, 4H), 3.45-3.35 (m, 4H), 2.37-2.24 (m, 1H), 2.19-2.04 |
| | | | | (m, 2H), 2.04-1.89 |
| | | | | (m, 4H), 1.89-1.73 |
| | | | | (m, 3H) |
| 862 | ((R)-fluoro(2- (((3S,6S,9aS)-3-(3- (4-((S)-2- (methoxymethyl) azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 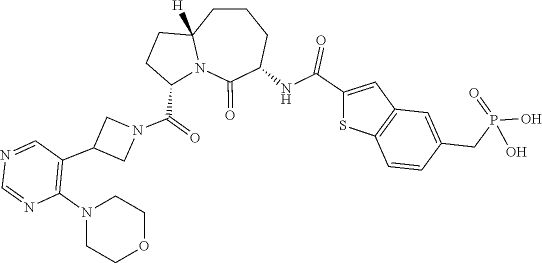 [SMILES: O=C(N[C@H]1CCC[C@H]2CC[C@@H](C(=O)N3CC(c4cncnc4N4CCOCC4)C3)N2C1=O)c1cc2cc(CP(=O)(O)O)ccc2s1] | 701.5 | ¹H NMR (400 MHz, CD₃OD) δ 8.34-8.10 (m, 1H), 8.09-7.92 (m, 3H), 7.83 (t, J = 8.4 Hz, 1H), 7.63- 7.46 (m, 1H), 5.84- 5.60 (m, 1H), 4.81- 4.65 (m, 3H), 4.63- 4.45 (m, 3H), 4.43- 4.20 (m, 2H), 4.13- 3.91 (m, 4H), 3.57- 3.51 (m, 1H), 3.36 (d, J = 14.0 Hz, 3H), 2.60- 2.51 (m, 1H), 2.45- 2.26 (m, 2H), 2.17- 2.01 (m, 3H), 1.98- |
| | | | | 1.74 (m, 6H) |
| 863 | ((2-(((3S,6S,9aS)-3- (3-(4- (dimethylamino) pyrimidin-5- yl)azetidine-1- carbonyl)-5- oxooctahydro-1H- pyrrolo[1,2- a]azepin-6- yl)carbamoyl)benzo [b]thiophen-5- yl)methyl) phosphonic acid | 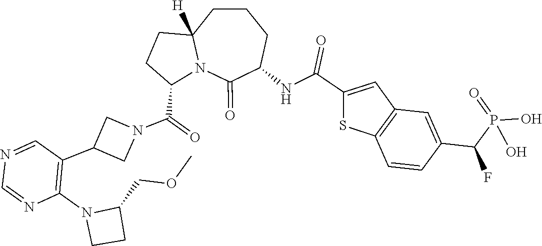 [SMILES: COC[C@@H]1CCN1c1ncncc1C1CN(C(=O)[C@@H]2CC[C@@H]3CCC[C@H](NC(=O)c4cc5cc([C@H](F)P(=O)(O)O)ccc5s4)C(=O)N32)C1] | 627.3 | 1H NMR (400 MHz, CD₃OD) δ 8.43-8.28 (m, 1H), 8.19 (s, 1H), 7.96 (d, J = 14.0 Hz, 1H), 7.83-7.71 (m, 2H), 7.46-7.33 (m, 1H), 4.77-4.60 (m, 2H), 4.57-4.42 (m, 2H), 4.40-4.16 (m, 2H), 4.13-3.95 (m, 2H), 3.24 (s, 6H), 3.20-3.11 (m, 2H), 2.39-2.24 (m, 1H), 2.21-2.06 (m, 2H), 2.05-1.79 (m, 7H) |
| 864 | ((6-fluoro-2- (((3S,6S,9aS)-5- oxo-3-(3-(pyridin-3- yl)azetidine-1- carbonyl)octahydro- 1H-pyrrolo[1,2- a]azepin-6- yl)carbamoyl)-1H- indol-5- yl)methyl) phosphonic acid | 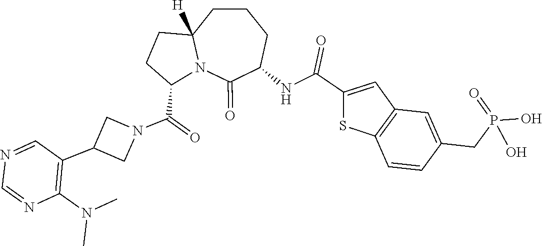 [SMILES: CN(C)c1ncncc1C1CN(C(=O)[C@@H]2CC[C@@H]3CCC[C@H](NC(=O)c4cc5cc(CP(=O)(O)O)ccc5s4)C(=O)N32)C1] | 584 | ¹H NMR (400 MHz, DMSO-d₆) δ 11.48 (s, 1H), 8.56 (d, J = 8.9 Hz, 1H), 8.51-8.36 (m, 2H), 7.91-7.81 (m, 1H), 7.64-7.52 (m, 1H), 7.45-7.31 (m, 1H), 7.22-7.14 (m, 1H), 7.11 (d, J = 10.4 Hz, 1H), 4.85- 4.61 (m, 2H), 4.45- 4.38 (m, 1H), 4.35- 4.18 (m, 2H), 4.07- 4.00 (m, 1H), 3.96- 3.89 (m, 2H), 3.04- |
| | | | | 2.98 (m, 2H), 2.23- |
| | | | | 2.13 (m, 1H), 2.07- |
| | | | | 1.94 (m, 2H), 1.87- |
| | | | | 1.66 (m, 7H) |
| 865 | ((2-(((3S,6S,9aS)-3- ((6S,7R)-7-cyano-6- phenyl-4- azaspiro[2.4] heptane-4- carbonyl)-5- oxooctahydro-1H- pyrrolo[1,2- a]azepin-6- yl)carbamoyl)benzo [b]thiophen-5- yl)difluoromethyl) phosphonic acid | 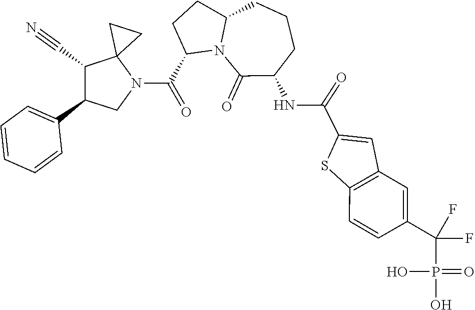 [SMILES: N#C[C@H]1[C@H](c2ccccc2)CN(C(=O)[C@@H]2CC[C@@H]3CCC[C@H](NC(=O)c4cc5cc(C(F)(F)P(=O)(O)O)ccc5s4)C(=O)N32)C12CC2] | 683.2 | ¹H NMR (400 MHz, DMSO-d⁶) δ 8.89- 8.79 (m, 1H), 8.34- 8.26 (m, 1H), 8.17- 8.04 (m, 2H), 7.64- 7.56 (m, 1H), 7.54- 7.47 (m, 2H), 7.45- 7.37 (m, 2H), 7.37- 7.30 (m, 1H), 4.69- 4.59 (m, 2H), 4.36- 4.33 (m, 1H), 4.02- 3.96 (m, 2H), 3.79- 3.67 (m, 2H), 2.27- 2.14 (m, 2H), 2.07- 1.92 (m, 2H), 1.89- 1.66 (m, 8H), 0.90- 0.78 (m, 1H), 0.76- 0.63 (m, 1H) |
| 866 | (difluoro(7- (((3S,6S,10aS)-5- oxo-3-(3-(2-oxo- 1,2-dihydropyridin- 4-yl)pyrrolidine-1- carbonyl)decahydro pyrrolo[1,2- a]azocin-6- yl)carbamoyl) naphthalen-2- yl)methyl) phosphonic acid | 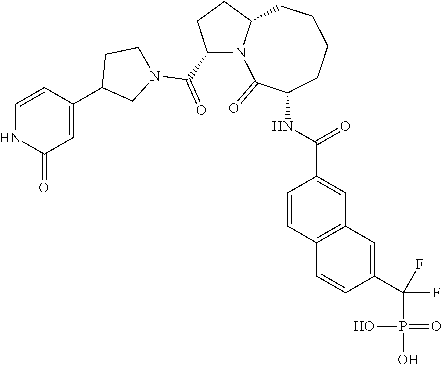 [SMILES: O=C(N[C@H]1CCCC[C@H]2CC[C@@H](C(=O)N3CCC(c4cc[nH]c(=O)c4)C3)N2C1=O)c1ccc2ccc(C(F)(F)P(=O)(O)O)cc2c1] | 657.4 | ¹H NMR (400 MHz, DMSO-d₆) δ 8.72- 8.58 (m, 2H), 8.20 (s, 1H), 8.13-7.98 (m, 3H), 7.72 (d, J = 8.6 Hz, 1H), 7.38-7.26 (m, 1H), 6.25-6.15 (m, 2H), 5.04-5.00 (m, 1H), 4.57 (m, 1H), 4.32 (m, 1H), 3.83 (m, 2H), 3.52- 3.07 (m, 3H), 2.28- 1.48 (m, 14H) |
| 867 | ((2-(((3S,6S,10aS)- 3-((rel-trans)-3- cyano-4-(6-oxo-1,6- dihydropyridin-2- yl)pyrrolidine-1- carbonyl)-5- oxodecahydropyrrolo [1,2-a]azocin-6- yl)carbamoyl)benzo [b]thiophen-5- yl)difluoromethyl) phosphonic acid | 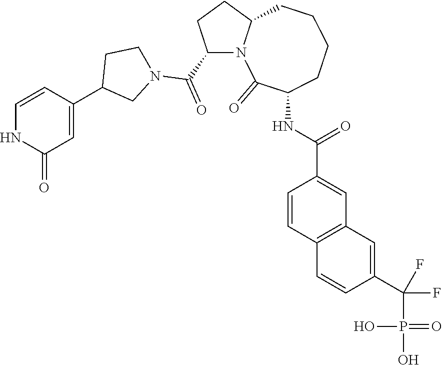 [SMILES: O=C(N[C@H]1CCCC[C@H]2CC[C@@H](C(=O)N3CCC(c4cc[nH]c(=O)c4)C3)N2C1=O)c1ccc2ccc(C(F)(F)P(=O)(O)O)cc2c1] | 688 | ¹H NMR (400 MHz, DMSO-d₆) δ 8.89- 8.79 (m, 1H), 8.35- 8.28 (m, 1H), 8.17- 8.10 (m, 1H), 8.07 (s, 1H), 7.59 (d, J = 8.5 Hz, 1H), 7.52-7.42 (m, 1H), 6.46-6.30 (m, 2H), 5.00-4.89 (m, 1H), 4.59-4.50 (m, 1H), 4.36-4.26 (m, 2H), 3.92-3.83 (m, 2H), 3.64-3.50 (m, 3H), 2.32-2.17 (m, 1H), 2.13-1.72 (m, 9H), 1.66-1.54 (m, 2H) |
| 868 | ((2-(((3S,6S,10aS)- 3-((rel-trans)-3- cyano-4-(2-oxo-1,2- dihydropyridin-3- yl)pyrrolidine-1- carbonyl)-5- oxodecahydropyrrol o[1,2-a]azocin-6- yl)carbamoyl)benzo [b]thiophen-5- yl)difluoromethyl) phosphonic acid | 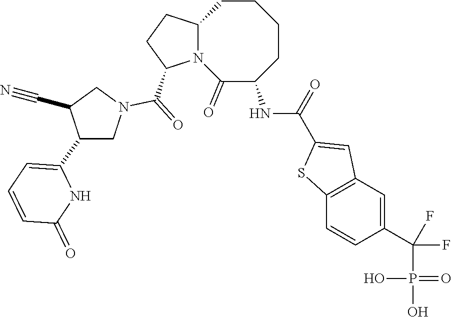 [SMILES: N#C[C@H]1CN(C(=O)[C@@H]2CC[C@@H]3CCCC[C@H](NC(=O)c4cc5cc(C(F)(F)P(=O)(O)O)ccc5s4)C(=O)N32)C[C@@H]1c1cccc(=O)[nH]1] | 688.3 | ¹H NMR (400 MHz, DMSO-d₆) δ 11.84 (s, 1H), 8.96-8.77 (m, 1H), 8.38-8.27 (m, 1H), 8.18-8.01 (m, 2H), 7.62-7.32 (m, 3H), 6.29-6.13 (m, 1H), 5.01-4.92 (m, 1H), 4.58-4.51 (m, 1H), 4.35-4.24 (m, 2H), 4.04-3.73 (m, 5H), 2.27-1.52 (m, 12H) |
| 869 | ((S)-(2- (((3S,6S,10aS)-3- ((3R,4S)-3-cyano-4- (2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)fluoromethyl) phosphonic acid Or ((S)-(2- (((3S,6S,10aS)-3- ((3S,4R)-3-cyano-4- (2-oxo-1,2- dihydropyridin-4- | 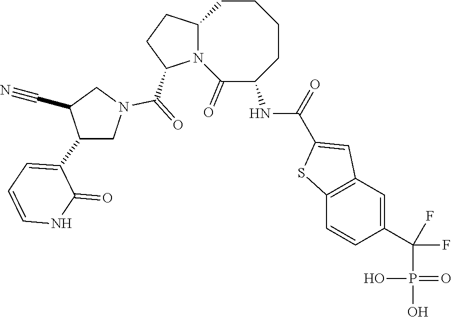 [SMILES: N#C[C@H]1CN(C(=O)[C@@H]2CC[C@@H]3CCCC[C@H](NC(=O)c4cc5cc(C(F)(F)P(=O)(O)O)ccc5s4)C(=O)N32)C[C@@H]1c1ccc[nH]c1=O] | 670 | ¹H NMR (400 MHz, DMSO-d₆) δ 8.80 (m, 1H), 8.27 (s, 1H), 8.03 (dd, J = 8.4, 3.0 Hz, 1H), 7.97 (s, 1H), 7.52 (d, J = 8.5 Hz, 1H), 7.39 (d, J = 6.8 Hz, 1H), 6.37 (d, J = 10.7 Hz, 1H), 6.29- 6.22 (m, 1H), 5.89- 5.72 (m, 1H), 5.00- 4.90 (m, 1H), 4.56 (m, 1H), 4.33-4.23 (m, 2H), 3.96-3.91 (m, 1H), 3.82-3.78 (m, 1H), 3.68-3.65 (m, 1H), 3.51-3.46 (m, 1H), 3.33-3.18 (m, 1H), 2.35-2.18 |
| | yl)pyrrolidine-1- | Pyrrolidine building block SFC | | (m, 1H), 2.05 (m, |
| | carbonyl)-5- | peak 1 was used for synthesis | | 1H), 2.01-1.70 (m, |
| | oxodecahydropyrrolo | and biological testing | | 7H), 1.70-1.44 (m, |
| | [1,2-a]azocin-6- | | | 3H) |
| | yl)carbamoyl)benzo | | | |
| | [b]thiophen-5- | | | |
| | yl)fluoromethyl) | | | |
| | phosphonic acid | | | |
| 870 | ((R)-fluoro(2- (((3S,6S,9aS)-5- oxo-3-(3-(pyridin-3- yl)azetidine-1- carbonyl)octahydro- 1H-pyrrolo[1,2- a]azepin-6- yl)carbamoyl)benzo [b]thiophen-5- yl)methyl) phosphonic acid | 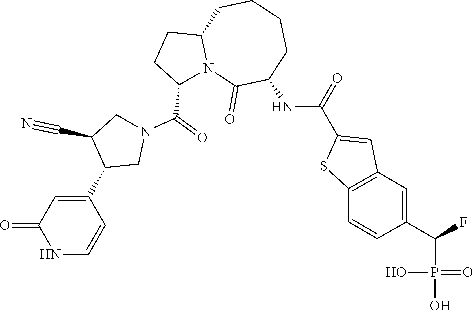 [SMILES: N#C[C@H]1CN(C(=O)[C@@H]2CC[C@@H]3CCCC[C@H](NC(=O)c4cc5cc([C@@H](F)P(=O)(O)O)ccc5s4)C(=O)N32)C[C@@H]1c1cc[nH]c(=O)c1] | 601.1 | ¹H NMR (400 MHz, DMSO-d₆) δ 8.88- 8.75 (m, 1H), 8.63- 8.44 (m, 2H), 8.27 (s, 1H), 8.17 (s, 1H), 8.00-7.96 (m, 1H), 7.93-7.84 (m, 1H), 7.58-7.53 (m, 1H), 7.46-7.34 (m, 1H), 5.89-5.76 (m, 1H), 4.73-4.57 (m, 2H), 4.48-4.36 (m, 2H), 4.35-4.19 (m, 2H), 4.04-3.94 (m, 2H), 2.24-2.15 (m, 1H), 2.03-1.72 (m, 9H) |
| | | or | | |
| | | 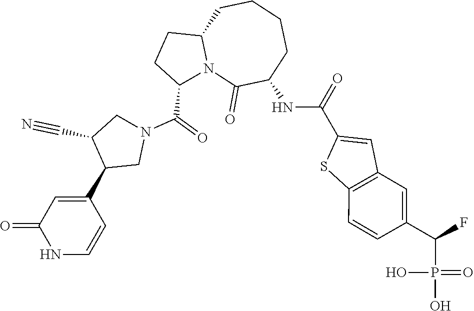 [SMILES: N#C[C@@H]1CN(C(=O)[C@@H]2CC[C@@H]3CCCC[C@H](NC(=O)c4cc5cc([C@@H](F)P(=O)(O)O)ccc5s4)C(=O)N32)C[C@H]1c1cc[nH]c(=O)c1] | | |
| 871 | (difluoro(7- (((3S,6S,10aS)-3- ((rel-trans)-3- hydroxy-4-(1H- 1,2,3-triazol-1- yl)pyrrolidine-1- carbonyl)-5- oxodecahydropyrrol o[1,2-a]azocin-6- yl)carbamoyl) naphthalen-2- yl)methyl) phosphonic acid | 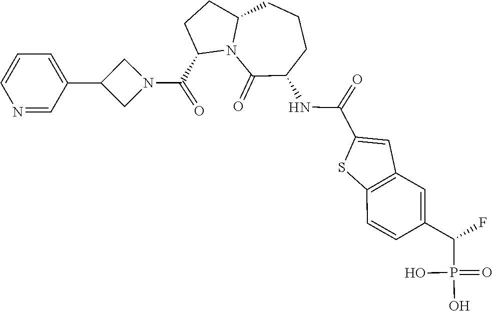 [SMILES: O=C(N[C@H]1CCC[C@H]2CC[C@@H](C(=O)N3CC(c4cccnc4)C3)N2C1=O)c1cc2cc([C@H](F)P(=O)(O)O)ccc2s1] | 647.0 | ¹H NMR (400 MHz, DMSO-d6) 8 8.76- 8.61 (m, 2H), 8.25- 7.99 (m, 6H), 7.80- 7.68 (m, 2H), 5.15- 4.94 (m, 2H), 4.63- 4.54 (m, 1H), 4.41- 4.29 (m, 2H), 3.96- 3.56 (m, 4H), 2.19- 1.63 (m, 12H) |
| 872 | ((R)-(2- (((3S,6S,10aS)-3- ((3S,4R)-3-cyano-4- (2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)fluoromethyl) phosphonic acid Or ((R)-(2- (((3S,6S,10aS)-3- ((3R,4S)-3-cyano-4- (2-oxo-1,2- dihydropyridin-4- yl)pyrrolidine-1- carbonyl)-5- oxodecahydropyrrolo [1,2-a]azocin-6- | 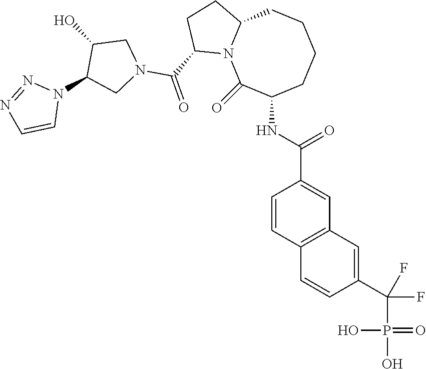 [SMILES: O=C(N[C@H]1CCCC[C@H]2CC[C@@H](C(=O)N3C[C@@H](O)[C@H](n4ccnn4)C3)N2C1=O)c1ccc2ccc(C(F)(F)P(=O)(O)O)cc2c1] | 670.2 | ¹H NMR (400 MHz, DMSO-d₆) δ 8.83- 8.75 (m, 1H), 8.26 (s, 1H), 8.02 (d, J = 8.4 Hz, 1H), 7.96 (s, 1H), 7.55-7.47 (m, 1H), 7.39 (d, J = 6.7 Hz, 1H), 6.40-6.32 (m, 1H), 6.28-6.20 (m, 1H), 5.77 (dd, J = 44.6, 7.7 Hz, 1H), 4.99-4.90 (m, 1H), 4.56 (t, J = 8.1 Hz, 1H), 4.35-4.25 (m, 2H), 3.83-3.58 (m, 4H), 3.24-3.08 (m, 1H), 2.32-2.17 (m, 1H), 2.06-1.73 (m, 8H), 1.69-1.49 (m, 3H |
| | yl)carbamoyl)benzo | | | |
| | [b]thiophen-5- | Pyrrolidine building block SFC | | |
| | yl)fluoromethyl) | peak 2 was used for synthesis | | |
| | phosphonic acid | and biological testing | | |
| 873 | ((S)-fluoro(2- (((3S,6S,9aS)-5- oxo-3-(3- (802yridine-3- yl)azetidine-1- carbonyl)octahydro- 1H-pyrrolo[1,2- a]azepin-6- yl)carbamoyl)benzo [b]thiophen-5- yl)methyl) phosphonic acid | 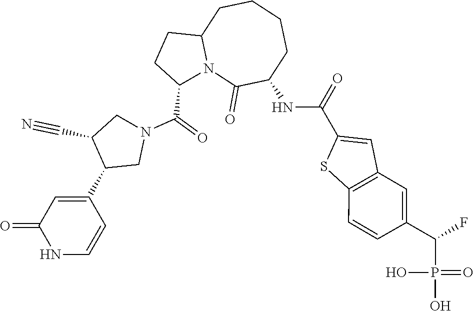 [SMILES: N#C[C@@H]1CN(C(=O)[C@@H]2CCC3CCCC[C@H](NC(=O)c4cc5cc([C@H](F)P(=O)(O)O)ccc5s4)C(=O)N32)C[C@@H]1c1cc[nH]c(=O)c1] | 601.2 | ¹H NMR (400 MHz, DMSO-d₆) δ 8.85- 8.74 (m, 1H), 8.62- 8.50 (m, 1H), 8.50- 8.42 (m, 1H), 8.26 (s, 1H), 8.04 (d, J = 8.5 Hz, 1H), 8.01-7.96 (m, 1H), 7.90-7.83 (m, 1H), 7.57-7.47 (m, 1H), 7.44-7.33 (m, 1H), 5.92-5.71 (m, 1H), 4.83-4.59 (m, 2H), 4.46-4.40 (m, 1H), 4.35-4.17 (m, 2H), 4.04-3.98 (m, 1H), 3.94-3.86 (m, 2H), 2.20 (d, J = 6.6 Hz, 1H), 2.09- 1.93 (m, 2H), 1.89- 1.66 (m, 7H) |
| | | or | | |
| | | 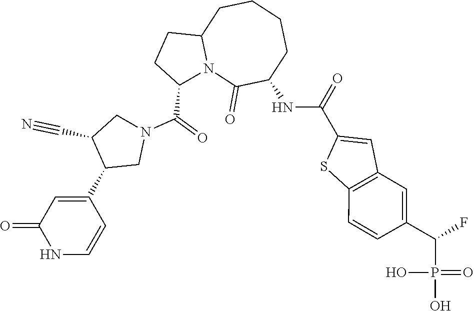 [SMILES: N#C[C@@H]1CN(C(=O)[C@@H]2CCC3CCCC[C@H](NC(=O)c4cc5cc([C@H](F)P(=O)(O)O)ccc5s4)C(=O)N32)C[C@@H]1c1cc[nH]c(=O)c1] | | |
| 874 | ((2-(((3S,6S,10aS)- 3-((rel-trans)-3- cyano-4-(2- methoxy-6- methylpyridin-4- yl)pyrrolidine-1- carbonyl)-5- oxodecahydropyrrol o[1,2-a]azocin-6- yl)carbamoyl)benzo [b]thiophen-5- yl)difluoromethyl) phosphonic acid | 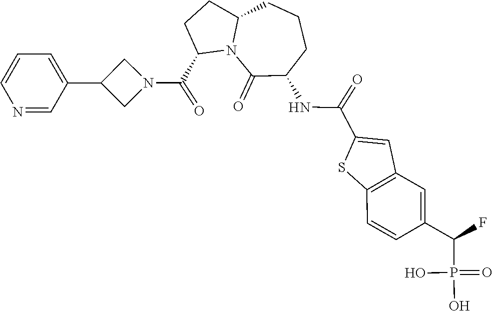 [SMILES: O=C(N[C@H]1CCC[C@H]2CC[C@@H](C(=O)N3CC(c4cccnc4)C3)N2C1=O)c1cc2cc([C@@H](F)P(=O)(O)O)ccc2s1] | 716.2 | ¹H NMR (400 MHz, DMSO-d₆) δ 9.07 8.51 (m, 1H), 8.41 8.25 (m, 1H), 8.21 8.10 (m, 1H), 8.08 (s, 1H), 7.59 (d, J = 8.5 Hz, 1H), 6.99-6.79 (m, 1H), 6.75-6.49 (m, 1H), 5.02-4.88 (m, 1H), 4.63-4.47 (m, 1H), 4.38-4.24 (m, 1H), 4.23-4.08 (m, 1H), 4.06-3.91 (m, 1H), 3.87-3.77 (m, 3H), 3.75-3.62 (m, 1H), 3.62 · 3.55 (m, 1H), 3.55-3.46 (m, 1H), 3.45-3.11 (m, 1H), 2.45-2.31 |
| | | | | (m, 3H), 2.32-2.16 |
| | | | | (m, 1H), 2.16-2.00 |
| | | | | (m, 1H), 2.00-1.71 |
| | | | | (m, 7H), 1.71-1.40 |
| | | | | (m, 3H) (TFA salt) |
| 875 | ((2-(((3S,6S,10aS)- 3-((3R,4S)-3-cyano- 4-(2-oxo-1,2- dihydropyridin-4- yl)pyrrolidine-1- carbonyl)-5- oxodecahydropyrrol o[1,2-a]azocin-6- yl)carbamoyl)benzo [b]thiophen-5- yl)difluoromethyl) phosphonic acid Or ((2-(((3S,6S,10aS)- 3-((3S,4R)-3-cyano- 4-(2-oxo-1,2- dihydropyridin-4- yl)pyrrolidine-1- carbonyl)-5- | 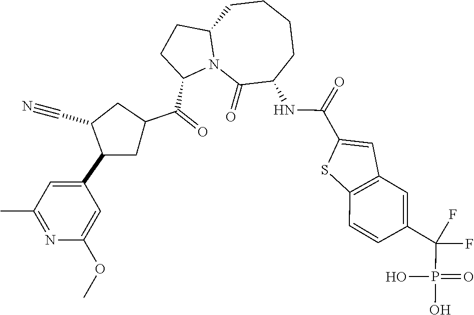 [SMILES: COc1cc([C@@H]2CC(C(=O)[C@@H]3CC[C@@H]4CCCC[C@H](NC(=O)c5cc6cc(C(F)(F)P(=O)(O)O)ccc6s5)C(=O)N43)C[C@H]2C#N)cc(C)n1] | 688.0 | H NMR (400 MHz, DMSO-d₆) δ 8.92- 8.81 (m, 1H), 8.32 (s, 1H), 8.18-8.10 (m, 1H), 8.07 (s, 1H), 7.58 (d, J = 8.5 Hz, 1H), 7.39 (d, J = 6.7 Hz, 1H), 6.42-6.19 (m, 2H), 5.01-4.88 (m, 1H), 4.62-4.50 (m, 1H), 4.37-4.20 (m, 2H), 4.01-3.76 (m, 2H), 3.72-3.59 (m, 1H), 3.55-3.44 (m, 1H), 3.25-3.02 (m, 1H), 2.35-2.16 (m, 1H), 2.14-1.99 (m, 1H), 1.97-1.69 (m, 7H), 1.69-1.41 |
| | oxodecahydropyrrol | | | (m, 3H) (free base) |
| | o[1,2-a]azocin-6- | Pyrrolidine building block SFC | | |
| | yl)carbamoyl)benzo | peak 1 was used for synthesis | | |
| | [b]thiophen-5- | and biological testing | | |
| | yl)difluoromethyl)p | | | |
| | hosphonic acid | | | |
| 876 | ((2-(((3S,6S,10aS)- 3-((rel-trans)-3- cyano-4-(6-methyl- 2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)difluoromethyl) phosphonic acid | 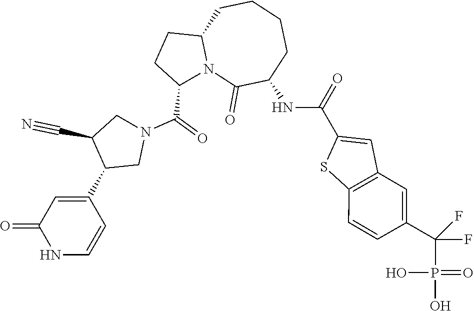 [SMILES: N#C[C@H]1CN(C(=O)[C@@H]2CC[C@@H]3CCCC[C@H](NC(=O)c4cc5cc(C(F)(F)P(=O)(O)O)ccc5s4)C(=O)N32)C[C@@H]1c1cc[nH]c(=O)c1] | 702.2 | ¹H NMR (400 MHz, DMSO-d₆) δ 9.00- 8.70 (m, 1H), 8.36- 8.22 (m, 1H), 8.08- 7.94 (m, 2H), 7.59 (d, J = 8.5 Hz, 1H), 6.23- 6.13 (m, 1H), 6.11- 5.91 (m, 1H), 5.07- 4.84 (m, 1H), 4.63- 4.43 (m, 1H), 4.37- 4.27 (m, 1H), 4.24- 4.11 (m, 1H), 4.09 3.98 (m, 1H), 3.96 3.85 (m, 1H), 3.85 3.76 (m, 1H), 3.31 3.14 (m, 2H), 2.34 2.22 (m, 1H), 2.18 2.11 (m, 3H), 2.10- 2.01 (m, 1H), 1.95- |
| | | | | 1.71 (m, 7H), 1.71- |
| | | or | | 1.48 (m, 3H). (free |
| | | | | base) |
| | | 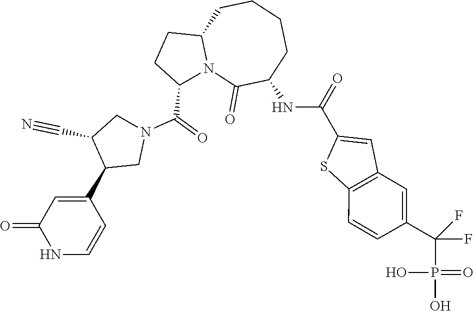 [SMILES: N#C[C@@H]1CN(C(=O)[C@@H]2CC[C@@H]3CCCC[C@H](NC(=O)c4cc5cc(C(F)(F)P(=O)(O)O)ccc5s4)C(=O)N32)C[C@H]1c1cc[nH]c(=O)c1] | | |
| 877 | (difluoro(7- (((3S,6S,10aS)-5- oxo-3-(6-(2-oxo- 1,2-dihydropyridin- 4-yl)-4- azaspiro[2.4] heptane-4- carbonyl)decahydro pyrrolo[1,2- a]azocin-6- yl)carbamoyl) naphthalen-2- yl)methyl) phosphonic acid | 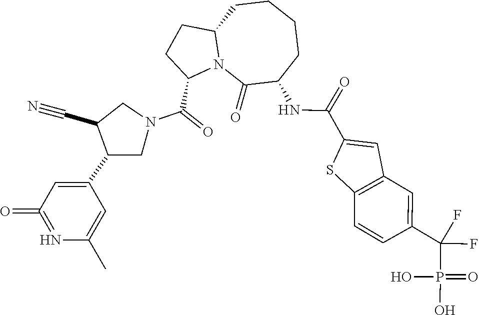 | 683.3 | ¹H NMR (400 MHz, DMSO-d₆) δ 8.77- 8.51 (m, 2H), 8.26- 8.11 (m, 1H), 8.07- 7.89 (m, 3H), 7.73 (s, 1H), 7.31-7.16 (m, 1H), 6.30-6.05 (m, 2H), 5.13-4.91 (m, 1H), 4.60-4.17 (m, 3H), 4.09--3.98 (m, 1H), 3.75-3.70 (m, 1H), 3.15-3.09 (m, 1H), 2.29-2.19 (m, 1H), 2.10-1.99 (m, 3H), 1.97-1.68 (m, 8H), 1.65-1.48 (m, 4H), 0.58-0.34 (m, 2H) |
| 878 | ((2-(((3S,6S,10aS)- 3-((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)methyl) phosphonic acid Or ((2-(((3S,6S,10aS)- 3-((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- | 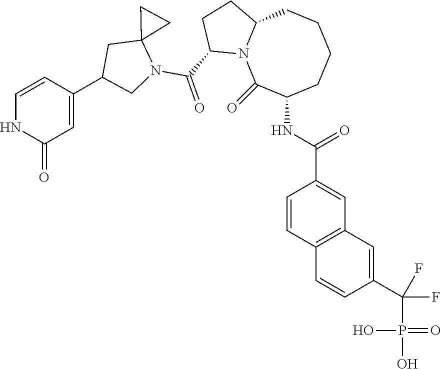 [SMILES: O=C(N[C@H]1CCCC[C@H]2CC[C@@H](C(=O)N3CC(c4cc[nH]c(=O)c4)CC34CC4)N2C1=O)c1ccc2ccc(C(F)(F)P(=O)(O)O)cc2c1] | 652.2 | ¹H NMR (400 MHz, DMSO-d₆) δ 8.80- 8.71 (m, 1H), 8.23- 8.15 (m, 1H), 7.94- 7.87 (m, 1H), 7.81- 7.76 (m, 1H), 7.41 7.33 (m, 2H), 6.40 6.32 (m, 1H), 6.28 6.22 (m, 1H), 4.97 4.90 (m, 1H), 4.59 4.52 (m, 1H), 4.33 4.24 (m, 2H), 3.91 3.75 (m, 2H), 3.63- 3.50 (m, 2H), 3.27- 3.14 (m, 1H), 3.13- 3.05 (m, 2H), 2.32 2.17 (m, 1H), 2.11 1.99 (m, 1H), 1.97- 1.70 (m, 7H), 1.66- 1.48 (m, 3H) |
| | yl)methyl) | | | |
| | phosphonic acid | Pyrrolidine building block SFC | | |
| | | peak 1 was used for synthesis | | |
| | | and biological testing | | |
| 879 | (difluoro(7- (((3S,6S,10aS)-3-(6- (1-methyl-2-oxo- 1,2-dihydropyridin- 4-yl)-4- azaspiro[2.4] heptane-4- carbonyl)-5- oxodecahydropyrrolo [1,2-a]azocin-6- yl)carbamoyl) naphthalen-2- yl)methyl) phosphonic acid | 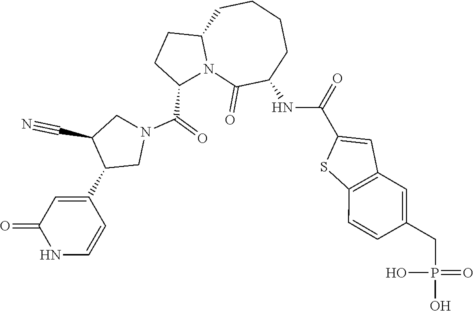 [SMILES: N#C[C@H]1CN(C(=O)[C@@H]2CC[C@@H]3CCCC[C@H](NC(=O)c4cc5cc(CP(=O)(O)O)ccc5s4)C(=O)N32)C[C@@H]1c1cc[nH]c(=O)c1] | 695.3 | ¹H NMR (400 MHz, DMSO-d₆) δ 8.81- 8.53 (m, 2H), 8.27- 8.11 (m, 1H), 8.10- 7.93 (m, 3H), 7.80- 7.65 (m, 1H), 7.66- 7.49 (m, 1H), 6.31 (s, 1H), 6.27-6.10 (m, 1H), 5.09-4.93 (m, 1H), 4.58-4.44 (m, 1H), 4.32-4.03 (m, 2H), 3.73-3.53 (m, 1H), 3.41-3.24 (m, 4H), 2.40-2.10 (m, 2H), 2.10-1.67 (m, 11H), 1.66-1.37 (m, 4H), 0.46 (s, 2H) |
| | | or | | |
| | | 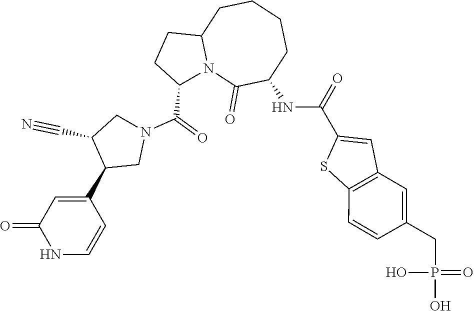 [SMILES: N#C[C@@H]1CN(C(=O)[C@@H]2CCC3CCCC[C@H](NC(=O)c4cc5cc(CP(=O)(O)O)ccc5s4)C(=O)N32)C[C@H]1c1cc[nH]c(=O)c1] | | |
| 880 | (difluoro(7- (((3S,6S,10aS)-5- oxo-3-(6-phenyl-4- azaspiro[2.4] heptane-4- carbonyl)decahydro pyrrolo[1,2- a]azocin-6- yl)carbamoyl) naphthalen-2- yl)methyl) phosphonic acid | 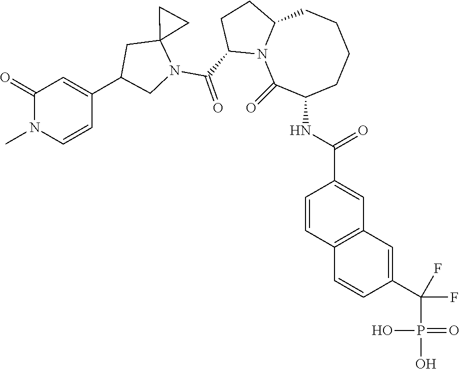 [SMILES: Cn1ccc(C2CN(C(=O)[C@@H]3CC[C@@H]4CCCC[C@H](NC(=O)c5ccc6ccc(C(F)(F)P(=O)(O)O)cc6c5)C(=O)N43)C3(CC3)C2)cc1=O] | 666.3 | ¹H NMR (400 MHz, DMSO-d₆) δ 8.78- 8.42 (m, 2H), 8.26- 7.97 (m, 4H), 7.89- 7.68 (m, 1H), 7.40- 7.11 (m, 5H), 5.07- 4.96 (m, 1H), 4.58 4.38 (m, 2H), 4.33 4.22 (m, 1H), 4.16 3.69 (m, 1H), 3.61 3.42 (m, 2H), 2.37- 1.43 (m, 16H), 0.91- 0.41 (m, 2H) |
| 881 | ((3-((E)-3- (((3S,6S,10aS)-3- ([1,1′-biphenyl]-4- ylcarbamoyl)-5- oxodecahydropyrrolo [1,2-a]azocin-6- yl)amino)-3- oxoprop-1-en-1- yl)phenyl) difluoromethyl) phosphonic acid | 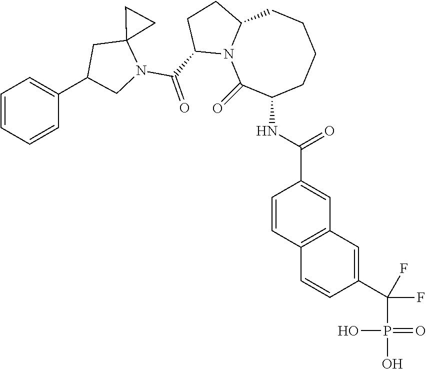 [SMILES: O=C(N[C@H]1CCCC[C@H]2CC[C@@H](C(=O)N3CC(c4ccccc4)CC34CC4)N2C1=O)c1ccc2ccc(C(F)(F)P(=O)(O)O)cc2c1] | 636.2 [M − H]⁻ | ¹H NMR (400 MHz, DMSO-d₆) δ 10.27- 9.96 (m, 1H), 8.39 8.31 (m, 1H), 7.75- 7.68 (m, 2H), 7.68 7.59 (m, 6H), 7.59 7.48 (m, 2H), 7.48 7.40 (m, 3H), 7.36- 7.29 (m, 1H), 6.86 (d, J = 15.8 Hz, 1H), 5.00- 4.81 (m, 1H), 4.55- 4.40 (m, 1H), 4.37- 4.21 (m, 1H), 3.68- 3.53 (m, 1H), 3.20 3.05 (m, 1H), 2.29 2.18 (m, 1H), 2.17 2.04 (m, 1H), 2.02 1.83 (m, 4H), 1.82- 1.71 (m, 2H), 1.71 1.58 (m, 3H), 1.57- 1.39 (m, 1H) |
| 882 | ((7-(((3S,6S,10aS)- 5-oxo-3-((S)-6-(2- oxo-1,2- dihydropyridin-4- yl)-4- azaspiro[2.4] heptane-4- carbonyl)decahydro pyrrolo[1,2- a]azocin-6- yl)carbamoyl) naphthalen-2- yl)methyl) phosphonic acid | 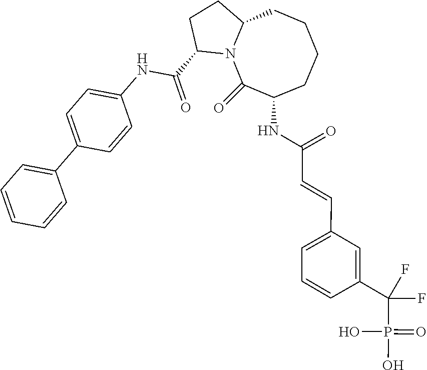 [SMILES: O=C(/C=C/c1cccc(C(F)(F)P(=O)(O)O)c1)N[C@H]1CCCC[C@H]2CC[C@@H](C(=O)Nc3ccc(-c4ccccc4)cc3)N2C1=O] | 647.4 | ¹H NMR (400 MHz, DMSO-d₆) δ 8.61 (d, J = 7.3 Hz, 1H), 8.45 (s, 1H), 7.90 (M, 4H), 7.52 (d, J = 8.4 Hz, 1H), 7.30 (d, J = 6.8 Hz, 1H), 6.25 (s, 1H), 6.19 (d, J = 6.6 Hz, 1H), 5.06-4.95 (m, 1H), 4.56-4.49 (m, 1H), 4.40-4.17 (m, 3H), 3.60-3.59 (m, 1H), 3.19-3.15 (m, 2H), 2.25-2.13 (m, 2H), 2.07-2.00 (m, 2H), 1.97-1.85 (m, 4H), 1.84-1.78 (m, 2H), 1.76-1.66 (m, 2H), 1.64-1.47 (m, 4H), 0.52-0.41 (m, 2H) |
| 883 | ((7-(((3S,6S,10aS)- 5-oxo-3-((R)-6-(2- oxo-1,2- dihydropyridin-4- yl)-4- azaspiro[2.4] heptane-4- carbonyl)decahydro pyrrolo[1,2- a]azocin-6- yl)carbamoyl) naphthalen-2- yl)methyl) phosphonic acid | 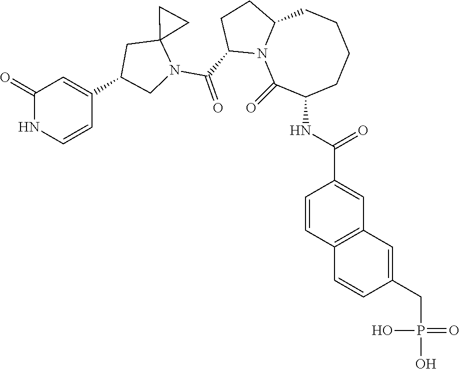 [SMILES: O=C(N[C@H]1CCCC[C@H]2CC[C@@H](C(=O)N3C[C@H](c4cc[nH]c(=O)c4)CC34CC4)N2C1=O)c1ccc2ccc(CP(=O)(O)O)cc2c1] | 647.4 | ¹H NMR (400 MHz, DMSO-d₆) δ 8.61 (d, J = 7.3 Hz, 1H), 8.44 (s, 1H), 8.00-7.79 (m, 4H), 7.52 (d, J = 8.4 Hz, 1H), 7.28 (d, J = 6.7 Hz, 1H), 6.26 (s, 1H), 6.21-6.16 (m, 1H), 5.06-4.94 (m, 1H), 4.53-4.44 (m, 1H), 4.31-4.23 (m, 1H), 4.09-4.02 (m, 1H), 3.74-3.68 (m, 2H), 3.18 3.14 (m, 2H), 2.36--2.18 (m, 2H), 2.12 1.96 (m, 5H), 1.92--1.81 (m, 3H), 1.78 1.69 (m, 2H), 1.66-1.60 (m, 1H), 1.58-1.48 (m, 3H), 0.53-0.40 (m, 2H) |
| 884 | ((7-(((3S,6S,10aS)- 5-oxo-3-(6-phenyl- 4-azaspiro[2.4] heptane-4- carbonyl)decahydro pyrrolo[1,2- a]azocin-6- yl)carbamoyl) naphthalen-2- yl)methyl) phosphonic acid | 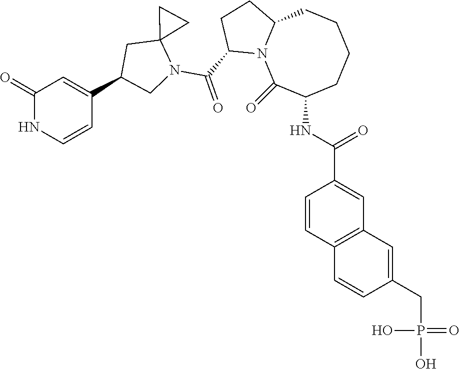 [SMILES: O=C(N[C@H]1CCCC[C@H]2CC[C@@H](C(=O)N3C[C@@H](c4cc[nH]c(=O)c4)CC34CC4)N2C1=O)c1ccc2ccc(CP(=O)(O)O)cc2c1] | 630.3 | ¹H NMR (400 MHz, DMSO-d₆) δ 8.65- 8.41 (m, 2H), 7.96- 7.80 (m, 4H), 7.55- 7.48 (m, 1H), 7.37- 7.17 (m, 5H), 5.06- 4.94 (m, 1H), 4.58 4.39 (m, 1H), 4.31- 4.23 (m, 1H), 4.16- 3.69 (m, 1H), 3.59- 3.47 (m, 2H), 3.16 (d, J = 21.4 Hz, 2H), 2.36- 2.16 (m, 2H), 2.13- 1.70 (m, 10H), 1.68- 1.46 (m, 4H), 0.55- 0.39 (m, 2H) |
| 885 | ((7- (((3S,6S,10aR,Z)-9- methyl-5-oxo-3-(6- phenyl-4- azaspiro[2.4] heptane-4- carbonyl)- 1,2,3,5,6,7,8,10a- octahydropyrrolo[1, 2-a]azocin-6- yl)carbamoyl) naphthalen-2- yl)methyl) phosphonic acid | 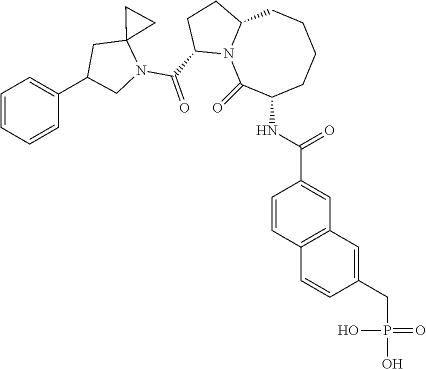 [SMILES: O=C(N[C@H]1CCCC[C@H]2CC[C@@H](C(=O)N3CC(c4ccccc4)CC34CC4)N2C1=O)c1ccc2ccc(CP(=O)(O)O)cc2c1] | 642.4 | ¹H NMR (400 MHz, DMSO-d₆) δ 8.82- 8.60 (m, 1H), 8.44 (s, 1H), 8.01-7.78 (m, 4H), 7.56-7.46 (m, 1H), 7.39-7.28 (m, 4H), 7.26-7.13 (m, 1H), 5.34 (s, 1H), 4.98-4.82 (m, 1H), 4.77-4.65 (m, 1H), 4.60-4.49 (m, 1H), 4.40-4.31 (m, 0.6 H), 3.72-3.67 (m, 0.4 H), 3.58-3.45 (m, 2H), 3.15 (d, J = 21.5 Hz, 2H), 2.38 2.18 (m, 2H), 2.13- 1.82 (m, 8H), 1.81- 1.73 (m, 3H), 1.66 1.45 (m, 2H), 0.59- 0.35 (m, 2H) |
| 886 | ((7- (((3S,6S,10aR,Z)-9- methyl-5-oxo-3-(6- phenyl-4- azaspiro[2.4] heptane-4- carbonyl)- 1,2,3,5,6,7,10,10a- octahydropyrrolo[1, 2-a]azocin-6- yl)carbamoyl) naphthalen-2- yl)methyl) phosphonic acid | 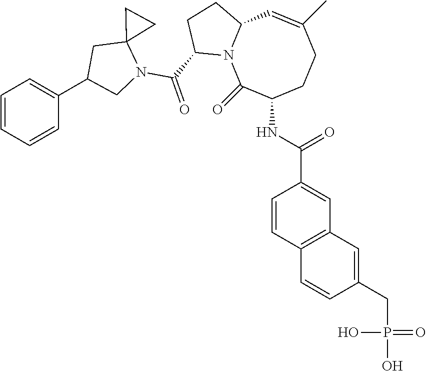 [SMILES: C/C1=C/[C@H]2CC[C@@H](C(=O)N3CC(c4ccccc4)CC34CC4)N2C(=O)[C@@H](NC(=O)c2ccc3ccc(CP(=O)(O)O)cc3c2)CC1] | 642.4 | ¹H NMR (400 MHz, DMSO-d₆) δ 8.66 (d, J = 7.5 Hz, 1H), 8.45 (s, 1H), 8.00-7.77 (m, 4H), 7.52 (d, J = 8.5 Hz, 1H), 7.42- 7.15 (m, 5H), 5.64 (t, J = 7.1 Hz, 1H), 5.03- 4.81 (m, 1H), 4.60- 4.49 (m, 1H), 4.44- 4.35 (m, 1H), 4.32 4.19 (m, 1H), 3.79- 3.64 (m, 2H), 3.15 (d, J = 21.4 Hz, 2H) , 2.67-2.54 (m, 2H), 2.46-2.38 (m, 1H), 2.36-2.26 (m, 2H), 2.22-2.11 (m, 3H), 2.09-1.90 (m, 2H), 1.81 (s, 3H), 1.78- 1.68 (m, 1H), 1.62- 1.52 (m, 1H), 0.64- 0.37 (m, 2H) |
| 887 | ((7- (((3S,6S,9R,10aR)- 9-methyl-5-oxo-3- (6-phenyl-4- azaspiro[2.4] heptane-4- carbonyl)decahydro pyrrolo[1,2- a]azocin-6- yl)carbamoyl) naphthalen-2- yl)methyl) phosphonic acid | 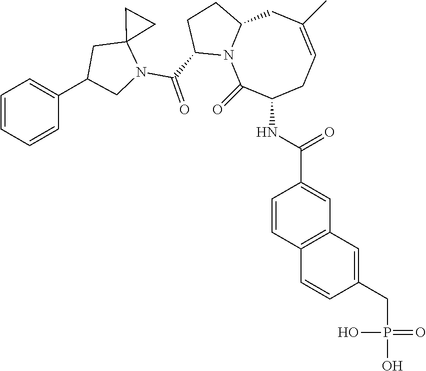 [SMILES: C/C1=C/C[C@H](NC(=O)c2ccc3ccc(CP(=O)(O)O)cc3c2)C(=O)N2[C@H](CC[C@H]2C(=O)N2CC(c3ccccc3)CC23CC3)C1] | 644.4 | ¹H NMR (400 MHz, DMSO-d₆) δ 8.77- 8.35 (m, 1H), 8.50- 8.36 (m, 1H), 8.06 7.74 (m, 4H), 7.55- 7.46 (m, 1H), 7.42- 7.10 (m, 5H), 5.76 (s, 1H), 5.21-4.93 (m, 1H), 4.66-4.45 (m, 1H), 4.38-4.22 (m, 1H), 4.14-4.10 (m, 0.5H), 3.73-3.71 (m, 0.5H), 3.62-3.48 (m, 1H), 3.11 (d, J = 21.4 Hz, 2H), 2.37-1.67 (m, 12H), 1.61-1.43 (m, 2H), 1.36-1.17 (m, 1H), 0.97 (d, J = 6.1 Hz, 3H), 0.58- 0.39 (m, 2H) |
| 888 | ((7- (((3S,6S,9S,10aR)- 9-methyl-5-oxo-3- (6-phenyl-4- azaspiro[2.4] heptane-4- carbonyl)decahydro pyrrolo[1,2- a]azocin-6- yl)carbamoyl) naphthalen-2- yl)methyl) phosphonic acid | 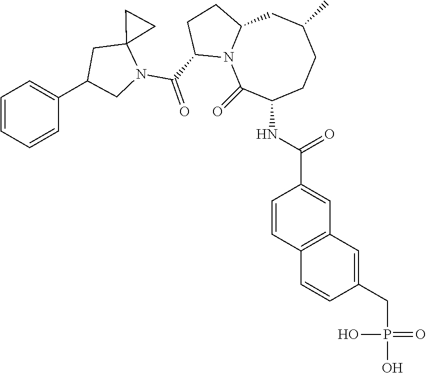 [SMILES: C[C@@H]1CC[C@H](NC(=O)c2ccc3ccc(CP(=O)(O)O)cc3c2)C(=O)N2[C@H](CC[C@H]2C(=O)N2CC(c3ccccc3)CC23CC3)C1] | 644.4 | ¹H NMR (400 MHz, DMSO-d₆) δ 8.60- 8.45 (m, 1H), 8.44- 8.36 (m, 1H), 7.94- 7.73 (m, 4H), 7.51 (d, J = 8.4 Hz, 1H), 7.36- 7.16 (m, 5H), 5.76 (s, 1H), 4.89-4.75 (m, 1H), 4.57-4.42 (m, 1H), 4.36-4.27 (m, 0.5 H), 4.15- 4.08 (m, 0.5 H), 3.76- 3.70 (m, 1H), 3.59- 3.53 (m, 1H), 3.11 (d, J = 21.4 Hz, 2H), 2.36- 2.21 (m, 2H), 2.12- 1.93 (m, 5H), 1.84- 1.49 (m, 7H), 1.25- 1.17 (m, 1H), 0.96 (d, J = 6.8 Hz, 3H), 0.55- 0.38 (m, 2H) |
| 889 | (difluoro(2- (((3S,6S,10aS)-5- oxo-3-((R)-6- phenyl-4- azaspiro[2.4] heptane-4- carbonyl)decahydro pyrrolo[1,2- a]azocin-6- yl)carbamoyl)benzo [b]thiophen-5- yl)methyl) phosphonic acid Or (difluoro(2- (((3S,6S,10aS)-5- oxo-3-((S)-6- phenyl-4- azaspiro[2.4] heptane-4- carbonyl)decahydro pyrrolo[1,2- a]azocin-6- | 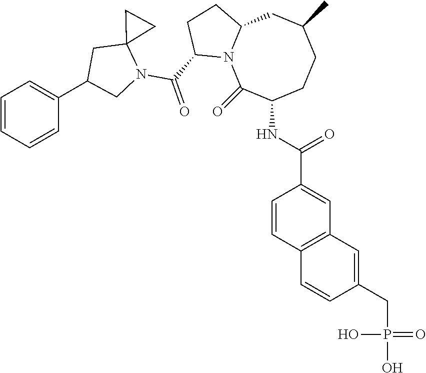 [SMILES: C[C@H]1CC[C@H](NC(=O)c2ccc3ccc(CP(=O)(O)O)cc3c2)C(=O)N2[C@H](CC[C@H]2C(=O)N2CC(c3ccccc3)CC23CC3)C1] | 672.1 | 1H NMR (400 MHz, DMSO-d₆) δ 8.88 (d, J = 7.3 Hz, 1H), 8.32 (s, 1H), 8.12 (d, J = 8.6 Hz, 1H), 8.06 (s, 1H), 7.58 (d, J = 8.5 Hz, 1H), 7.37-7.27 (m, 4H), 7.26-7.20 (m, 1H), 5.02-4.91 (m, 1H), 4.58-4.49 (m, 1H), 4.47-4.39 (m, 1H), 4.26-4.19 (m, 1H), 3.89-3.79 (m, 2H), 2.28-1.55 (m, 16H), 0.52-0.43 (m, 2H) |
| | yl)carbamoyl)benzo | | | |
| | [b]thiophen-5- | Pyrrolidine building block SFC | | |
| | yl)methyl) | peak 2 was used for synthesis | | |
| | phosphonic acid | and biological testing | | |
| 890 | (difluoro(2- (((3S,6S,10aS)-5- oxo-3-((R)-6- phenyl-4- azaspiro[2.4] heptane-4- carbonyl)decahydro pyrrolo[1,2- a]azocin-6- yl)carbamoyl)benzo [b]thiophen-5- yl)methyl) phosphonic acid Or (difluoro(2- (((3S,6S,10aS)-5- oxo-3-((S)-6- phenyl-4- azaspiro[2.4] heptane-4- carbonyl)decahydro | 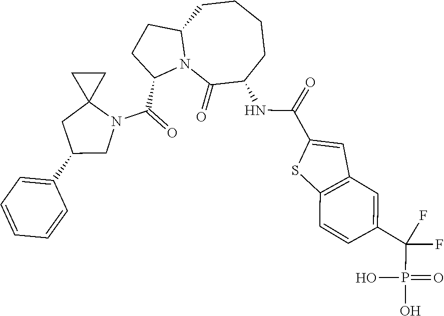 [SMILES: O=C(N[C@H]1CCCC[C@H]2CC[C@@H](C(=O)N3C[C@H](c4ccccc4)CC34CC4)N2C1=O)c1cc2cc(C(F)(F)P(=O)(O)O)ccc2s1] | 672.1 | ¹H NMR (400 MHz, DMSO-d₆) δ 8.86 (d, J = 7.1 Hz, 1H), 8.31 (s, 1H), 8.11 (d, J = 8.5 Hz, 1H), 8.05 (s, 1H), 7.57 (d, J = 8.3 Hz, 1H), 7.38-7.27 (m, 4H), 7.25-7.18 (m, 1H), 5.02-4.90 (m, 1H), 4.56-4.45 (m, 1H), 4.30-4.19 (m, 1H), 4.16-4.08 (m, 1H), 3.80-3.69 (m, 1H), 3.56-3.50 (m, 1H), 2.27-1.55 (m, 16H), 0.53-0.40 (m, 2H) |
| | pyrrolo[1,2- | | | |
| | a]azocin-6- | or | | |
| | yl)carbamoyl)benzo | | | |
| | [b]thiophen-5- yl)methyl) phosphonic acid | 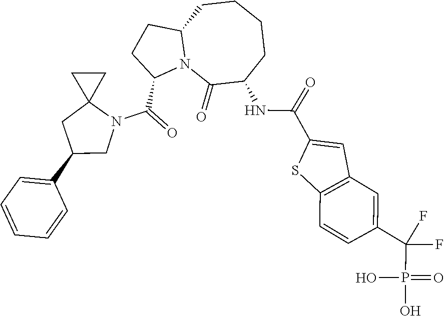 [SMILES: O=C(N[C@H]1CCCC[C@H]2CC[C@@H](C(=O)N3C[C@@H](c4ccccc4)CC34CC4)N2C1=O)c1cc2cc(C(F)(F)P(=O)(O)O)ccc2s1] | | |
| | | Pyrrolidine building block SFC | | |
| | | peak 1 was used for synthesis | | |
| | | and biological testing | | |
| 891 | (fluoro(7- (((3S,6S,10aS)-5- oxo-3-(6-(pyridin-3- yl)-4- azaspiro[2.4] heptane-4- carbonyl)decahydro pyrrolo[1,2- a]azocin-6- yl)carbamoyl) naphthalen-2- yl)methyl) phosphonic acid | 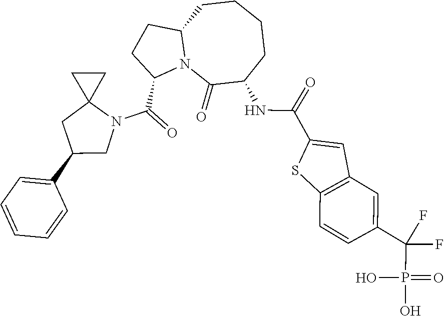 [SMILES: O=C(N[C@H]1CCCC[C@H]2CC[C@@H](C(=O)N3C[C@@H](c4ccccc4)CC34CC4)N2C1=O)c1cc2cc(C(F)(F)P(=O)(O)O)ccc2s1] | 649.2 | ¹H NMR (400 MHz, DMSO-d₆) δ 8.73- 8.37 (m, 4H), 8.21- 7.92 (m, 4H), 7.86 7.75 (m, 1H), 7.72 7.58 (m, 1H), 7.45 7.25 (m, 1H), 6.04 5.73 (m, 1H), 5.09 4.91 (m, 1H), 4.61- 4.04 (m, 3H), 3.88 3.41 (m, 2H), 2.36- 1.73 (m, 12H), 1.69 1.46 (m, 4H), 0.56- 0.37 (m, 2H) |
| | | or | | |
| | | 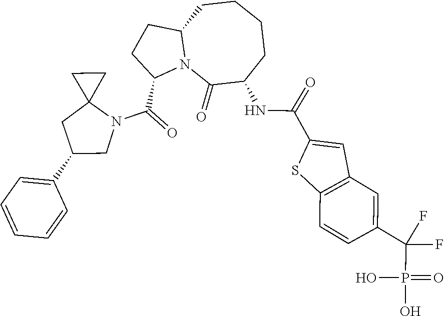 [SMILES: O=C(N[C@H]1CCCC[C@H]2CC[C@@H](C(=O)N3C[C@H](c4ccccc4)CC34CC4)N2C1=O)c1cc2cc(C(F)(F)P(=O)(O)O)ccc2s1] | | |
| 892 | (fluoro(7- (((3S,6S,10aS)-5- oxo-3-(3-(pyridin-3- yl)pyrrolidine-1- carbonyl)decahydro pyrrolo[1,2- a]azocin-6- yl)carbamoyl) naphthalen-2- yl)methyl) phosphonic acid | 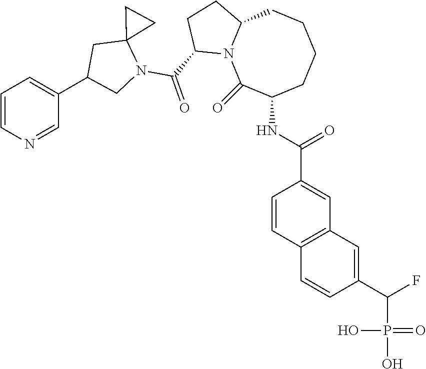 [SMILES: O=C(N[C@H]1CCCC[C@H]2CC[C@@H](C(=O)N3CC(c4cccnc4)CC34CC4)N2C1=O)c1ccc2ccc(C(F)P(=O)(O)O)cc2c1] | 623.4 | ¹H NMR (400 MHz, DMSO-d₆) δ 8.67- 8.57 (m, 1H), 8.56- 8.47 (m, 2H), 8.46- 8.38 (m, 1H), 8.01- 7.87 (m, 4H), 7.76 7.68 (m, 1H), 7.66 7.56 (m, 1H), 7.38 7.27 (m, 1H), 5.79- 5.58 (m, 1H), 5.07- 4.95 (m, 1H), 4.64- 4.51 (m, 1H), 4.37 4.29 (m, 1H), 4.01- 3.88 (m, 1H), 3.61- 3.45 (m, 3H), 3.13 3.03 (m, 1H), 2.39 2.19 (m, 2H), 2.10- 1.72 (m, 9H), 1.69 1.46 (m, 3H) |
| 893 | ((7-(((3S,6S,10aS)- 3-(5,7-dioxo-6- (pyridin-3-yl)-4,6- diazaspiro[2.4] heptane-4- carbonyl)-5- oxodecahydropyrrol o[1,2-a]azocin-6- yl)carbamoyl) naphthalen-2- yl)methyl) phosphonic acid | 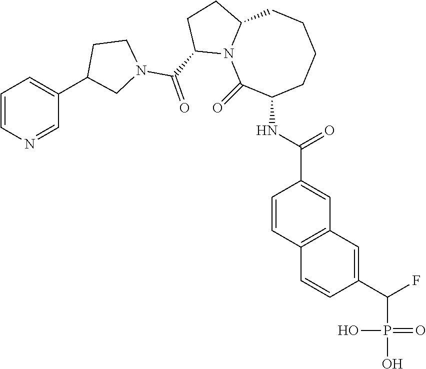 [SMILES: O=C(N[C@H]1CCCC[C@H]2CC[C@@H](C(=O)N3CCC(c4cccnc4)C3)N2C1=O)c1ccc2ccc(C(F)P(=O)(O)O)cc2c1] | 660.1 | ¹H NMR (400 MHz, DMSO-d6) 8 8.72- 8.39 (m, 4H), 7.98- 7.79 (m, 5H), 7.62- 7.47 (m, 2H), 5.38 (t, J = 8.6 Hz, 1H), 5.03- 4.92 (m, 1H), 4.45- 4.34 (m, 1H), 3.20- 3.07 (m, 2H), 2.46- 2.31 (m, 2H), 2.26- 2.04 (m, 2H), 1.98- 1.52 (m, 10H), 1.45- 1.34 (m, 2H) |
| 894 | ((7-(((3S,6S,10aS)- 3-((6S,7R)-7-cyano- 6-phenyl-4- azaspiro[2.4] heptane-4- carbonyl)-5- oxodecahydropyrrolo [1,2-a]azocin-6- yl)carbamoyl) naphthalen-2- yl)fluoromethyl) phosphonic acid | 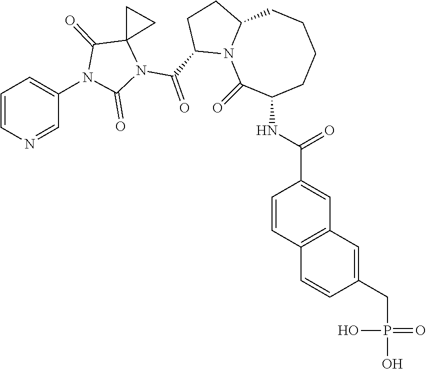 [SMILES: O=C(N[C@H]1CCCC[C@H]2CC[C@@H](C(=O)N3C(=O)N(c4cccnc4)C(=O)C34CC4)N2C1=O)c1ccc2ccc(CP(=O)(O)O)cc2c1] | 673.2 | ¹H NMR (400 MHz, DMSO-d₆) δ 8.75- 8.63 (m, 1H), 8.59- 8.49 (m, 1H), 8.10- 7.91 (m, 4H), 7.73- 7.62 (m, 1H), 7.62 7.42 (m, 2H), 7.40 7.30 (m, 2H), 5.92 (dd, J = 44.4, 8.5 Hz, 1H), 5.10-4.90 (m, 1H), 4.56-4.47 (m, 1H), 4.32-4.19 (m, 1H), 4.08-3.97 (m, 1H), 3.90-3.69 (m, 2H), 3.27-3.00 (m, 1H), 2.30-1.48 (m, 12H), 1.31-1.21 (m, 2H), 0.90-0.76 (m, 1H), 0.75-0.55 (m, 1H) (TFA salt) |
| 895 | ((3-(((3S,6S,10aS)- 5-oxo-3-(6-phenyl- 4-azaspiro[2.4] heptane-4- carbonyl)decahydro pyrrolo[1,2- a]azocin-6- yl)carbamoyl) quinolin-6- yl)methyl) phosphonic acid | 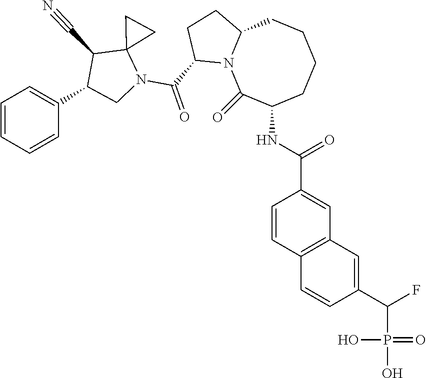 [SMILES: N#C[C@@H]1[C@@H](c2ccccc2)CN(C(=O)[C@@H]2CC[C@@H]3CCCC[C@H](NC(=O)c4ccc5ccc(C(F)P(=O)(O)O)cc5c4)C(=O)N32)C12CC2] | 631.3 | ¹H NMR (400 MHz, DMSO-d₆) δ 9.26- 9.16 (m, 1H), 8.87 (t, J = 7.3 Hz, 1H), 8.83- 8.77 (m, 1H), 7.98 (d, J = 8.6 Hz, 1H), 7.90-7.83 (m, 1H), 7.76 (d, J = 8.7 Hz, 1H), 7.37-7.26 (m, 4H), 7.25-7.18 (m, 1H), 5.10-4.96 (m, 1H), 4.60-4.47 (m, 1H), 4.46-4.09 (m, 2H), 3.74-3.57 (m, 1H), 3.53-3.44 (m, 1H), 3.18 (d, J = 21.5 Hz, 2H), 2.36-2.16 (m, 2H), 2.14-1.42 (m, 14H), 0.54-0.40 (m, 2H) |
| 896 | (1-(2-(((3S,6S,9aS)- 5-oxo-3-(3-(pyridin- 3-yl)azetidine-1- carbonyl)octahydro- 1H-pyrrolo[1,2- a]azepin-6- yl)carbamoyl)benzo [b]thiophen-5- yl)ethyl) phosphonic acid | 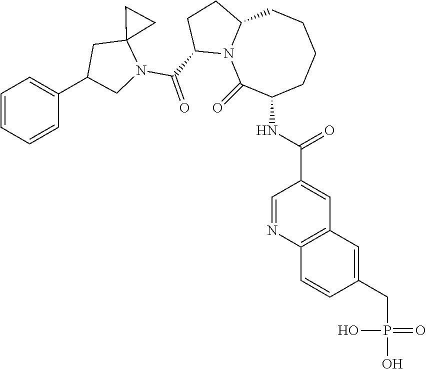 [SMILES: O=C(N[C@H]1CCCC[C@H]2CC[C@@H](C(=O)N3CC(c4ccccc4)CC34CC4)N2C1=O)c1cnc2ccc(CP(=O)(O)O)cc2c1] | 597.2 | ¹H NMR (400 MHz, DMSO-d₆) δ 8.78 8.68 (m, 1H), 8.59- 8.53 (m, 1H), 8.50- 8.41 (m, 1H), 8.18 (s, 1H), 7.93-7.78 (m, 3H), 7.45-7.32 (m, 2H), 4.81-4.60 (m, 2H), 4.49-4.36 (m, 2H), 4.34-4.20 (m, 2H), 4.02-3.98 (m, 1H), 3.94-3.90 (m, 1H), 3.19-3.09 (m, 1H), 2.25 2.14 (m, 1H), 2.08 1.94 (m, 2H), 1.88 1.64 (m, 7H), 1.53-1.44 (m, 3H) |
| 897 | (1-(7- (((3S,6S,10aS)-5- oxo-3-((R)-6- phenyl-4- azaspiro[2.4] heptane-4- carbonyl)decahydro pyrrolo[1,2- a]azocin-6- yl)carbamoyl) naphthalen-2- yl)ethyl)phosphonic acid Or (1-(7- (((3S,6S,10aS)-5- oxo-3-((S)-6- phenyl-4- azaspiro[2.4] heptane-4- | 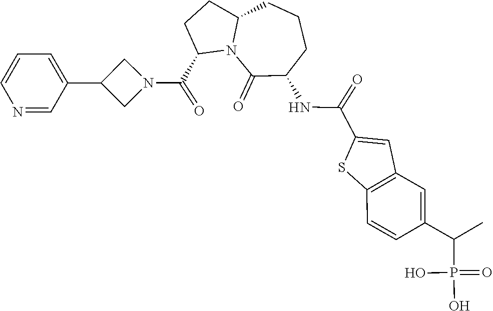 [SMILES: CC(c1ccc2sc(C(=O)N[C@H]3CCC[C@H]4CC[C@@H](C(=O)N5CC(c6cccnc6)C5)N4C3=O)cc2c1)P(=O)(O)O] | 644.4 | ¹H NMR (400 MHz, DMSO-d₆) δ 8.58 (d, J = 7.3 Hz, 1H), 8.47 (s, 1H), 7.94-7.82 (m, 4H), 7.58 (d, J = 8.6 Hz, 1H), 7.37- 7.26 (m, 4H), 7.24- 7.18 (m, 1H), 5.08 4.90 (m, 1H), 4.49 (t, J = 8.2 Hz, 1H), 4.33- 4.21 (m, 1H), 4.12 (t, J = 8.7 Hz, 1H), 3.73 (t, J = 9.9 Hz, 1H), 3.58-3.48 (m, 1H), 3.23-3.15 (m, 1H), 2.36--2.17 (m, 2H), 2.15-2.08 (m, 1H), 2.06-1.94 (m, 3H), 1.94 1.69 (m, 6H), 1.67--1.59 (m, |
| | carbonyl)decahydro | | | 1H), 1.58-1.45 (m, |
| | pyrrolo[1,2- | Pyrrolidine building block SFC | | 6H), 0.56-0.39 (m, |
| | a]azocin-6- | peak 1 was used for synthesis | | 2H) |
| | yl)carbamoyl) | and biological testing | | |
| | naphthalen-2- | | | |
| | yl)ethyl)phosphonic | | | |
| | acid | | | |
| 898 | (fluoro(2- (((3S,6S,10aS)-3-(6- (1-methyl-2-oxo- 1,2-dihydropyridin- 4-yl)-4- azaspiro[2.4] heptane-4- carbonyl)-5- oxodecahydropyrrolo [1,2-a]azocin-6- yl)carbamoyl)benzo [b]thiophen-5- yl)methyl) phosphonic acid | 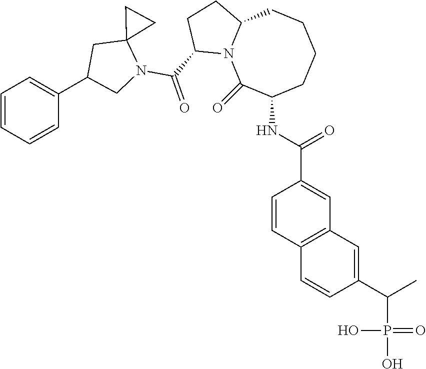 [SMILES: CC(c1ccc2ccc(C(=O)N[C@H]3CCCC[C@H]4CC[C@@H](C(=O)N5CC(c6ccccc6)CC56CC6)N4C3=O)cc2c1)P(=O)(O)O] | 685.3 | ¹H NMR (400 MHz, DMSO-d₆) δ 8.83 (d, J = 7.3 Hz, 1H), 8.27 (s, 1H), 8.03 (d, J = 8.5 Hz, 1H), 7.97 (s, 1H), 7.61 (t, J = 7.6 Hz, 1H), 7.53 (d, J = 8.6 Hz, 1H), 6.32 (s, 1H), 6.26-6.16 (m, 1H), 5.83 (dd, J = 44.3, 8.0 Hz, 1H), 5.00-4.85 (m, 1H), 4.59-4.44 (m, 1H), 4.38-4.14 (m, 2H), 4.10-4.00 (m, 1H), 3.74-3.68 (m, 0.5H), 3.59-3.55 (m, 0.5H), 3.37-3.34 (m, 3H), 2.31-2.10 (m, 2H), 2.10-1.97 (m, 3H), 1.96-1.67 (m, 7H), 1.66-1.41 (m, 4H), 0.57-0.37 (m, 2H). |
| | | or | | |
| | | 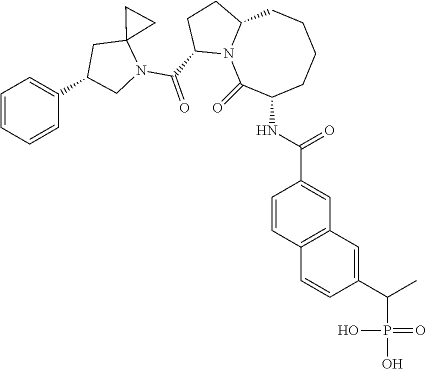 [SMILES: CC(c1ccc2ccc(C(=O)N[C@H]3CCCC[C@H]4CC[C@@H](C(=O)N5C[C@H](c6ccccc6)CC56CC6)N4C3=O)cc2c1)P(=O)(O)O] | | |
| 899 | (fluoro(7- (((3S,6S,10aS)-3-(3- (1-methyl-2-oxo- 1,2-dihydropyridin- 4-yl)pyrrolidine-1- carbonyl)-5- oxodecahydropyrrolo [1,2-a]azocin-6- yl)carbamoyl) naphthalen-2- yl)methyl) phosphonic acid | 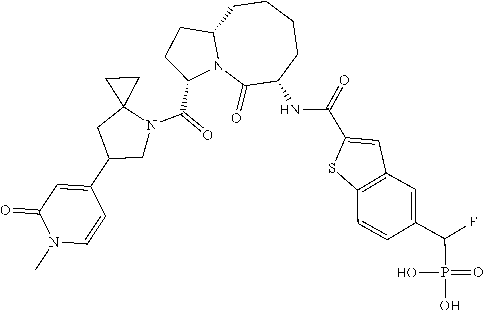 [SMILES: Cn1ccc(C2CN(C(=O)[C@@H]3CC[C@@H]4CCCC[C@H](NC(=O)c5cc6cc(C(F)P(=O)(O)O)ccc6s5)C(=O)N43)C3(CC3)C2)cc1=O] | 651.2 [M − H]⁻ | ¹H NMR (400 MHz, DMSO-d₆) δ 8.75 8.41 (m, 2H), 8.17- 7.85 (m, 4H), 7.71 7.47 (m, 2H), 6.33 6.10 (m, 2H), 5.95- 5.75 (m, 1H), 5.07 4.94 (m, 1H), 4.64 4.49 (m, 1H), 4.39 4.27 (m, 1H), 4.20 3.87 (m, 1H), 3.85 3.39 (m, 3H), 3.36 3.06 (m, 4H), 2.35 1.47 (m, 14H) |
| 900 | ((2-(((3S,6S,10aS)- 3-(5,7-dioxo-6- (pyridin-3-yl)-4,6- diazaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 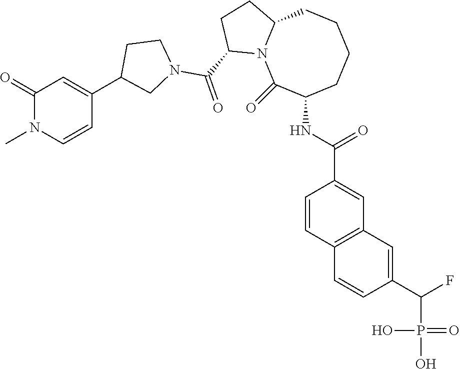 [SMILES: Cn1ccc(C2CCN(C(=O)[C@@H]3CC[C@@H]4CCCC[C@H](NC(=O)c5ccc6ccc(C(F)P(=O)(O)O)cc6c5)C(=O)N43)C2)cc1=O] | 684.1 | ¹H NMR (400 MHz, DMSO-d₆) δ 8.91- 8.60 (m, 3H), 8.30 8.19 (m, 1H), 8.07 7.88 (m, 3H), 7.63 7.46 (m, 2H), 5.88 5.69 (m, 1H), 5.44 5.31 (m, 1H), 4.98 4.86 (m, 1H), 4.40 4.30 (m, 1H), 2.45- 2.32 (m, 2H), 2.24 2.06 (m, 2H), 1.96 1.53 (m, 10H), 1.44 1.32 (m, 2H) |
| 901 | (methoxy(2- (((3S,6S,9aS)-5- oxo-3-(3-(pyridin-3- yl)azetidine-1- carbonyl)octahydro- 1H-pyrrolo[1,2- a]azepin-6- yl)carbamoyl)benzo [b]thiophen-5- yl)methyl) phosphonic acid | 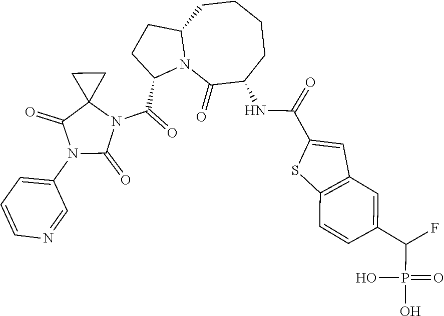 [SMILES: O=C(N[C@H]1CCCC[C@H]2CC[C@@H](C(=O)N3C(=O)N(c4cccnc4)C(=O)C34CC4)N2C1=O)c1cc2cc(C(F)P(=O)(O)O)ccc2s1] | 680.2 | ¹H NMR (400 MHz, DMSO-d₆) δ 8.81- 8.64 (m, 1H), 8.56 (s, 1H), 8.47 (d, J = 13.3 Hz, 1H), 8.23 (s, 1H), 7.97 (d, J = 8.1 Hz, 1H), 7.92-7.81 (m, 2H), 7.46 (d, J = 8.0 Hz, 1H), 7.43-7.30 (m, 1H), 4.87-4.54 (m, 2H), 4.52-4.19 (m, 4H), 4.04-3.84 (m, 3H), 3.28 (s, 3H), 2.25-2.11 (m, 1H), 2.10-1.91 (m, 2H), 1.91-1.61 (m, 7H). |
| 902 | (fluoro(7- (((3S,6S,10aS)-3-(3- (1-methyl-6-oxo- 1,6- dihydropyridazin-4- yl)pyrrolidine-1- carbonyl)-5- oxodecahydropyrrolo [1,2-a]azocin-6- yl)carbamoyl) naphthalen-2- yl)methyl) phosphonic acid | 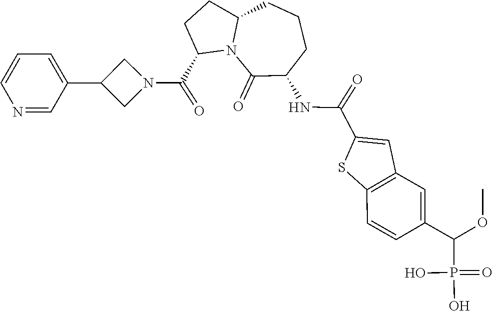 [SMILES: COC(c1ccc2sc(C(=O)N[C@H]3CCC[C@H]4CC[C@@H](C(=O)N5CC(c6cccnc6)C5)N4C3=O)cc2c1)P(=O)(O)O] | 654.2 | ¹H NMR (400 MHz, DMSO-d₆) δ 8.75- 8.42 (m, 2H), 8.21 7.76 (m, 5H), 7.71 7.51 (m, 1H), 6.86 6.69 (m, 1H), 5.93 5.62 (m, 1H), 5.09 4.91 (m, 1H), 4.63 4.50 (m, 1H), 4.39 4.30 (m, 1H), 3.98 3.90 (m, 1H), 3.77- 3.71 (m, 1H), 3.61 (s, 3H), 3.54-3.51 (m, 1H), 3.37-3.32 (m, 1H), 2.33-2.16 (m, 2H), 2.11-1.98 (m, 2H), 1.95-1.72 (m, 6H), 1.71-1.13 (m, 5H) |
| 903 | (fluoro(7- (((3S,6S,10aS)-3-(3- fluoro-3-(1-methyl- 2-oxo-1,2- dihydropyridin-4- yl)pyrrolidine-1- carbonyl)-5- oxodecahydropyrrolo [1,2-a]azocin-6- yl)carbamoyl) naphthalen-2- yl)methyl) phosphonic acid | 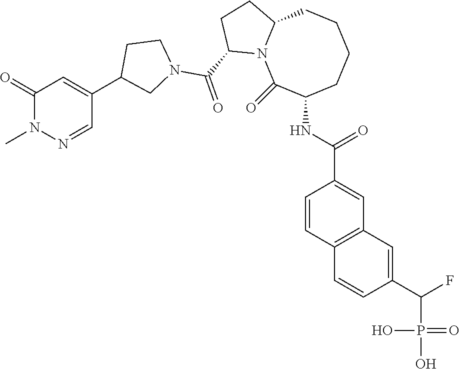 [SMILES: Cn1ncc(C2CCN(C(=O)[C@@H]3CC[C@@H]4CCCC[C@H](NC(=O)c5ccc6ccc(C(F)P(=O)(O)O)cc6c5)C(=O)N43)C2)cc1=O] | 671.3 | ¹H NMR (400 MHz, DMSO-d₆) δ 8.73- 8.42 (m, 2H), 8.15- 7.86 (m, 4H), 7.80- 7.57 (m, 2H), 6.53- 6.22 (m, 2H), 5.86- 5.66 (m, 1H), 5.02 (s, 1H), 4.63-4.50 (m, 1H), 4.36-4.31 (m, 1H), 4.16-4.09 (m, 1H), 3.92-3.87 (m, 1H), 3.72-3.65 (m, 1H), 3.59-3.50 (m, 1H), 3.42-3.36 (m, 3H), 2.40-2.22 (m, 2H), 2.12-1.77 (m, 8H), 1.74 1.44 (m, 4H) |
| 904 | (fluoro(7- (((3S,6S,9aS)-5- oxo-3-(6-(pyridin-3- yl)-4- azaspiro[2.4] heptane-4- carbonyl)octahydro- 1H-pyrrolo[1,2- a]azepin-6- yl)carbamoyl) naphthalen-2- yl)methyl) phosphonic acid | 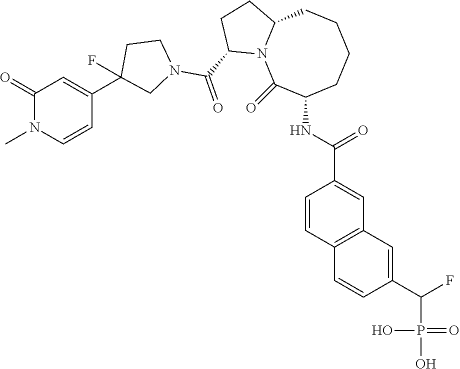 [SMILES: Cn1ccc(C2(F)CCN(C(=O)[C@@H]3CC[C@@H]4CCCC[C@H](NC(=O)c5ccc6ccc(C(F)P(=O)(O)O)cc6c5)C(=O)N43)C2)cc1=O] | 635.3 | ¹H NMR (400 MHz, DMSO-d₆) δ 8.68 8.39 (m, 4H), 8.17- 7.91 (m, 4H), 7.88 7.78 (m, 1H), 7.70 7.61 (m, 1H), 7.43- 7.30 (m, 1H), 5.94 5.79 (m, 1H), 4.69 4.66 (m, 1H), 4.36 4.27 (m, 1H), 4.11 3.93 (m, 2H), 3.78 3.54 (m, 2H), 2.44 1.22 (m, 14H), 0.92 0.43 (m, 2H) |
| 905 | (fluoro(2- (((3S,6S,10aS)-3-(3- (1-methyl-2-oxo- 1,2-dihydropyridin- 4-yl)pyrrolidine-1- carbonyl)-5- oxodecahydropyrrolo [1,2-a]azocin-6- yl)carbamoyl)benzo [b]thiophen-5- yl)methyl) phosphonic acid | 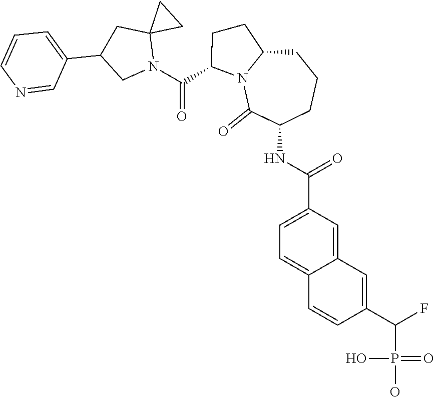 | 659.1 | |
| 906 | ((7-(((3S,6S,10aS)- 3-(3-(1-cyclopropyl- 2-oxo-1,2- dihydropyridin-4- yl)pyrrolidine-1- carbonyl)-5- oxodecahydropyrrol o[1,2-a]azocin-6- yl)carbamoyl) naphthalen-2- yl)fluoromethyl) phosphonic acid | 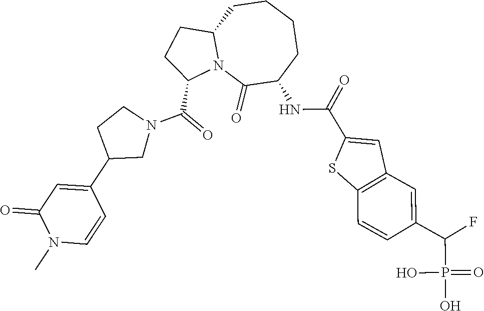 [SMILES: Cn1ccc(C2CCN(C(=O)[C@@H]3CC[C@@H]4CCCC[C@H](NC(=O)c5cc6cc(C(F)P(=O)(O)O)ccc6s5)C(=O)N43)C2)cc1=O] | 679.3 | ¹H NMR (400 MHz, DMSO-d₆) δ 8.69- 8.58 (m, 1H), 8.55 (s, 1H), 8.08-7.93 (m, 4H), 7.67 (d, J = 8.3 Hz, 1H), 7.48-7.40 (m, 1H), 6.24 (dd, J = 44.4, 8.4 Hz, 1H), 6.18-6.11 (m, 1H), 5.86 (d, J = 8.2 Hz, 1H), 5.07-4.96 (m, 1H), 4.63-4.50 (m, 1H), 4.39-4.27 (m, 1H), 4.17-3.77 (m, 1H), 3.76-3.36 (m, 2H), 3.35-3.21 (m, 2H), 3.21-3.03 (m, 1H), 2.34-1.74 (m, 11H), 1.68-1.47 (m, 3H), 1.01-0.87 (m, 2H), 0.83-0.71 (m, 2H) |
| 907 | (fluoro(2- (((3S,6S,9aS)-5- oxo-3-(6-(pyridin-3- yl)-4- azaspiro[2.4] heptane-4- carbonyl)octahydro- 1H-pyrrolo[1,2- a]azepin-6- yl)carbamoyl)benzo [b]thiophen-5- yl)methyl) phosphonic acid | 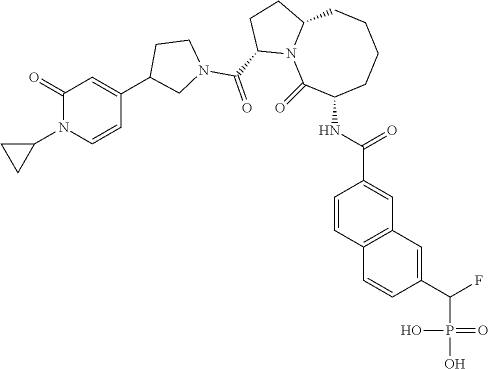 [SMILES: O=C(N[C@H]1CCCC[C@H]2CC[C@@H](C(=O)N3CCC(c4ccn(C5CC5)c(=O)c4)C3)N2C1=O)c1ccc2ccc(C(F)P(=O)(O)O)cc2c1] | 641.3 | ¹H NMR (400 MHz, DMSO-d₆) δ 8.80- 8.70 (m, 1H), 8.64- 8.40 (m, 2H), 8.26 8.13 (m, 1H), 8.07- 7.91 (m, 2H), 7.88- 7.76 (m, 1H), 7.57- 7.47 (m, 1H), 7.42- 7.30 (m, 1H), 5.79 (dd, J = 44.0, 7.6 Hz, 1H), 4.67-4.62 (m, 1H), 4.36-4.28 (m, 1H), 4.10-3.94 (m, 2H), 3.76-3.58 (m, 2H), 2.41-1.50 (m, 14H), 0.56-0.40 (m, 2H) |
| 908 | ((R)-fluoro(2- (((3S,6S,10aS)-5- oxo-3-((R)-6- (pyridin-3-yl)-4- azaspiro[2.4] heptane-4- carbonyl)decahydro pyrrolo[1,2- a]azocin-6- yl)carbamoyl)benzo [b]thiophen-5- yl)methyl) phosphonic acid | 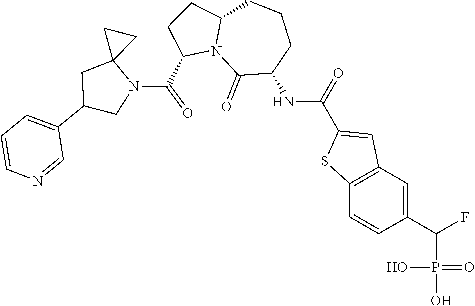 [SMILES: O=C(N[C@H]1CCC[C@H]2CC[C@@H](C(=O)N3CC(c4cccnc4)CC34CC4)N2C1=O)c1cc2cc(C(F)P(=O)(O)O)ccc2s1] | 655.2 | ¹H NMR (400 MHz, DMSO-d₆) δ 8.93- 8.75 (m, 1H), 8.61- 8.53 (m, 1H), 8.47- 8.40 (m, 1H), 8.26 (s, 1H), 8.07-7.91 (m, 2H), 7.84-7.76 (m, 1H), 7.56-7.47 (m, 1H), 7.43-7.30 (m, 1H), 5.89-5.70 (m, 1H), 5.00-4.88 (m, 1H), 4.57-4.49 (m, 1H), 4.25-4.12 (m, 1H), 3.85 3.77 (m, 1H), 3.62-3.53 (m, 1H), 2.34-1.45 (m, 17H), 0.55-0.39 (m, 2H) |
| 909 | ((S)-fluoro(2- (((3S,6S,10aS)-5- oxo-3-((S)-6- (pyridin-3-yl)-4- azaspiro[2.4] heptane-4- carbonyl)decahydro pyrrolo[1,2- a]azocin-6- yl)carbamoyl)benzo [b]thiophen-5- yl)methyl) phosphonic acid | 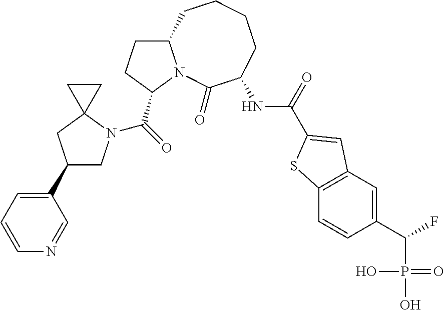 [SMILES: O=C(N[C@H]1CCCC[C@H]2CC[C@@H](C(=O)N3C[C@@H](c4cccnc4)CC34CC4)N2C1=O)c1cc2cc([C@H](F)P(=O)(O)O)ccc2s1] | 655.2 | ¹H NMR (400 MHz, DMSO-d₆) δ 8.83 (d, J = 7.3 Hz, 1H), 8.57 (d, J = 1.9 Hz, 1H), 8.45 (dd, J = 4.7, 1.4 Hz, 1H), 8.27 (s, 1H), 8.02 (d, J = 8.4 Hz, 1H), 7.97 (s, 1H), 7.85-7.77 (m, 1H), 7.52 (d, J = 8.5 Hz, 1H), 7.37 (dd, J = 7.9, 4.8 Hz, 1H), 5.92- 5.69 (m, 1H), 5.02- 4.87 (m, 1H), 4.54 (t, J = 8.4 Hz, 1H), 4.45 (t, J = 8.6 Hz, 1H), 4.27-4.18 (m, 1H), 3.66-3.59 (m, 1H), 3.58-3.48 (m, 1H), |
| | | | | 2.37-2.27 (m, 1H), |
| | | | | 2.24-2.15 (m, 1H), |
| | | | | 2.13-1.40 (m, 14H), |
| | | | | 0.56-0.41 (m, 2H) |
| 910 | ((S)-fluoro(2- (((3S,6S,10aS)-5- oxo-3-((R)-6- (pyridin-3-yl)-4- azaspiro[2.4] heptane-4- carbonyl)decahydro pyrrolo[1,2- a]azocin-6- yl)carbamoyl)benzo [b]thiophen-5- yl)methyl) phosphonic acid | 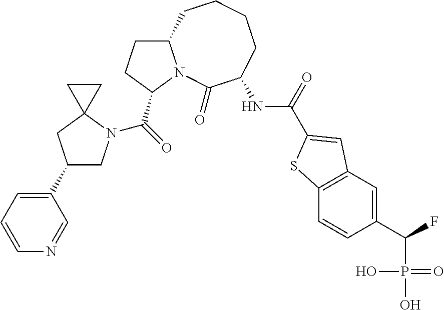 [SMILES: O=C(N[C@H]1CCCC[C@H]2CC[C@@H](C(=O)N3C[C@H](c4cccnc4)CC34CC4)N2C1=O)c1cc2cc([C@@H](F)P(=O)(O)O)ccc2s1] | 655.2 | ¹H NMR (400 MHz, DMSO-d₆) δ 8.83- 8.22 (m, 3H), 8.26 (s, 1H), 8.03-7.93 (m, 2H), 7.84-7.77 (m, 1H), 7.54-7.30 (m, 2H), 5.87-5.72 (m, 1H), 4.98-4.91 (m, 1H), 4.54-4.49 (m, 1H), 4.26-4.12 (m, 1H), 3.84-3.79 (m, 1H), 3.63--3.53 (m, 1H), 2.34-2.09 (m, 3H), 2.06-1.48 (m, 14H), 0.55-0.38 (m, 2H) |
| 911 | ((R)-fluoro(2- (((3S,6S,10aS)-5- oxo-3-((S)-6- (pyridin-3-yl)-4- azaspiro[2.4] heptane-4- carbonyl)decahydro pyrrolo[1,2- a]azocin-6- yl)carbamoyl)benzo [b]thiophen-5- yl)methyl) phosphonic acid | 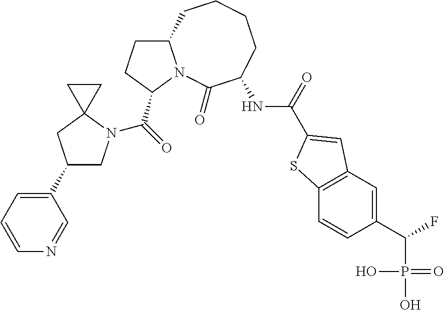 [SMILES: O=C(N[C@H]1CCCC[C@H]2CC[C@@H](C(=O)N3C[C@H](c4cccnc4)CC34CC4)N2C1=O)c1cc2cc([C@H](F)P(=O)(O)O)ccc2s1] | 655.2 | ¹H NMR (400 MHz, DMSO-d₆) δ 8.83 (d, J = 7.3 Hz, 1H), 8.59 (d, J = 2.0 Hz, 1H), 8.48 (dd, J = 4.8, 1.5 Hz, 1H), 8.28 (s, 1H), 8.03 (d, J = 8.5 Hz, 1H), 7.99-7.94 (m, 1H), 7.90-7.81 (m, 1H), 7.53 (d, J = 8.5 Hz, 1H), 7.41 (dd, J = 7.8, 4.8 Hz, 1H), 5.83 (dd, J = 44.3, 8.0 Hz, 1H), 5.01-4.89 (m, 1H), 4.57-4.50 (m, 1H), 4.49-4.42 (m, 1H), 4.27--4.20 (m, 1H), 3.67-3.59 (m, 1H), 3.58--3.50 (m, |
| | | | | 1H), 2.37-2.27 (m, |
| | | | | 1H), 2.25-2.15 (m, |
| | | | | 1H), 2.13-1.43 (m, |
| | | | | 14H), 0.57-0.42 (m, |
| | | | | 2H) |
| 912 | (fluoro(2- (((3S,6S,10aS)-5- oxo-3-(6-(pyridin-3- yl)-4- azaspiro[2.4] heptane-4- carbonyl)decahydro pyrrolo[1,2- a]azocin-6- yl)carbamoyl)benzo [b]thiophen-5- yl)methyl) phosphonic acid | 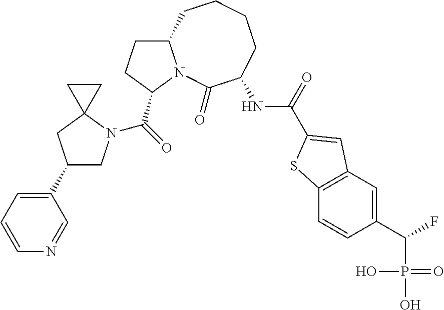 [SMILES: O=C(N[C@H]1CCCC[C@H]2CC[C@@H](C(=O)N3C[C@H](c4cccnc4)CC34CC4)N2C1=O)c1cc2cc([C@H](F)P(=O)(O)O)ccc2s1] | 655.2 | ¹H NMR (400 MHz, DMSO-d₆) δ 8.86- 8.79 (m, 1H), 8.69 8.60 (m, 1H), 8.58- 8.49 (m, 1H), 8.32- 8.24 (m, 1H), 8.09 7.94 (m, 3H), 7.60 7.47 (m, 2H), 5.92 5.76 (m, 1H), 5.00- 4.87 (m, 1H), 4.58- 4.42 (m, 2H), 4.26 4.18 (m, 1H), 3.67 3.60 (m, 2H), 2.35- 2.30 (m, 1H), 2.24 2.17 (m, 1H), 2.11 2.05 (m, 1H), 2.04 1.96 (m, 2H), 1.94 1.73 (m, 6H), 1.70- 1.46 (m, 5H), 0.58 0.41 (m, 2H) |
| 913 | (fluoro(2- (((3S,6S,10aS)-3-(3- (1-methyl-6-oxo- 1,6- dihydropyridazin-4- yl)pyrrolidine-1- carbonyl)-5- oxodecahydropyrrolo [1,2-a]azocin-6- yl)carbamoyl)benzo [b]thiophen-5- yl)methyl) phosphonic acid | 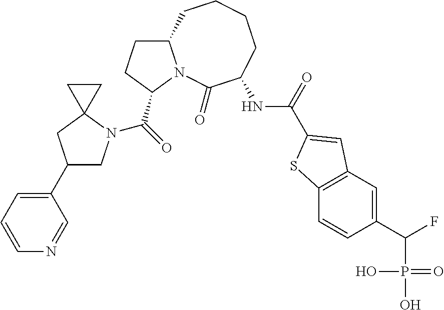 [SMILES: O=C(N[C@H]1CCCC[C@H]2CC[C@@H](C(=O)N3CC(c4cccnc4)CC34CC4)N2C1=O)c1cc2cc(C(F)P(=O)(O)O)ccc2s1] | 660.2 | ¹H NMR (400 MHz, DMSO-d₆) δ 8.92- 8.20 (m, 2H), 8.15- 7.41 (m, 4H), 6.91 6.62 (m, 1H), 6.00- 5.66 (m, 1H), 5.01 4.88 (m, 1H), 4.61 4.49 (m, 1H), 4.34 4.20 (m, 1H), 3.98 3.88 (m, 1H), 3.76 3.68 (m, 1H), 3.63 3.58 (m, 3H), 3.53 3.46 (m, 1H), 3.35 3.28 (m, 1H), 3.24 3.11 (m, 1H), 2.40 1.45 (m, 14H) |
| 914 | (fluoro(2- (((3S,6S,10aS)-3-(3- hydroxy-3-(1- methyl-2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)methyl) phosphonic acid | 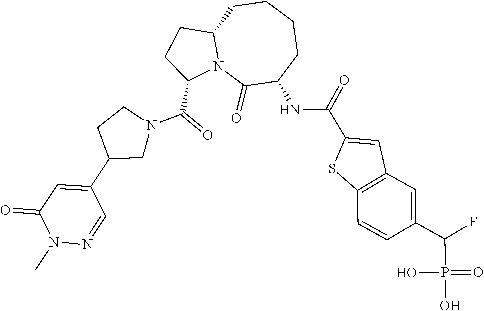 [SMILES: Cn1ncc(C2CCN(C(=O)[C@@H]3CC[C@@H]4CCCC[C@H](NC(=O)c5cc6cc(C(F)P(=O)(O)O)ccc6s5)C(=O)N43)C2)cc1=O] | 675.2 | ¹H NMR (400 MHz, DMSO-d₆) δ 8.82- 8.66 (m, 1H), 8.30 8.19 (m, 1H), 8.07- 7.87 (m, 2H), 7.66 7.45 (m, 2H), 6.55 6.43 (m, 1H), 6.36 6.22 (m, 1H), 5.87 5.69 (m, 1H), 5.68- 5.39 (m, 1H), 4.99 4.89 (m, 1H), 4.65 4.45 (m, 1H), 4.31 4.22 (m, 1H), 4.03 3.97 (m, 1H), 3.86- 3.81 (m, 1H), 3.74 3.69 (m, 1H), 3.59 3.54 (m, 1H), 3.39 3.34 (m, 3H), 2.28- 1.71 (m, 11H), 1.69- 1.46 (m, 3H) |
| 915 | ((R)-(2- (((3S,6S,9aS)-3- (5,7-dioxo-6- (pyridin-3-yl)-4,6- diazaspiro[2.4] heptane-4-carbonyl)-5- oxooctahydro-1H- pyrrolo[1,2- a]azepin-6- yl)carbamoyl)benzo [b]thiophen-5- yl)fluoromethyl) phosphonic acid | 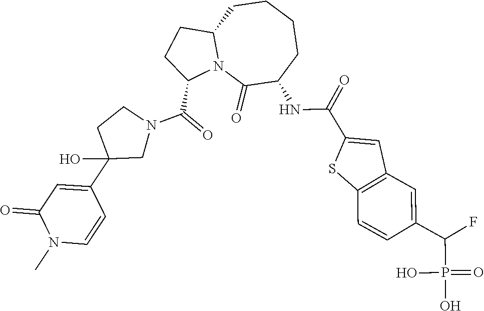 [SMILES: Cn1ccc(C2(O)CCN(C(=O)[C@@H]3CC[C@@H]4CCCC[C@H](NC(=O)c5cc6cc(C(F)P(=O)(O)O)ccc6s5)C(=O)N43)C2)cc1=O] | 670.1 | ¹H NMR (400 MHz, DMSO-d₆) δ 8.81 8.61 (m, 3H), 8.22 (s, 1H), 8.07-7.92 (m, 3H), 7.66-7.49 (m, 2H), 5.92-5.74 (m, 1H), 5.53-5.48 (m, 1H), 4.67-4.60 (m, 1H), 4.07-3.99 (m, 1H), 2.33-1.55 (m, 12H), 1.44-1.34 (m, 2H) |
| 916 | ((R)-(2- (((3S,6S,9aS)-3- ((6R,7S)-7-cyano-6- phenyl-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid | 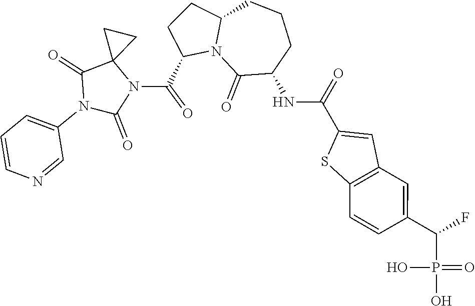 [SMILES: O=C(N[C@H]1CCC[C@H]2CC[C@@H](C(=O)N3C(=O)N(c4cccnc4)C(=O)C34CC4)N2C1=O)c1cc2cc([C@H](F)P(=O)(O)O)ccc2s1] | 665.3 | ¹H NMR (400 MHz, DMSO) δ 8.77 (d, J = 7.6 Hz, 1H), 8.24 (s, 1H), 8.07-7.94 (m, 2H), 7.56-7.46 (m, 3H), 7.43-7.27 (m, 3H), 5.83 (dd, J = 44.3, 8.0 Hz, 1H), 4.65-4.59 (m, 2H), 4.17-4.10 (m, 1H), 3.99-3.90 (m, 2H), 3.86-3.76 (m, 2H), 2.26-2.01 (m, 3H), 2.00-1.91 (m, 1H), 1.90-1.60 (m, 8H), 0.89-0.57 (m, 2H) |
| 917 | ((S)-fluoro(2- (((3S,6S,9aS)-5- oxo-3-((R)-6- (pyridin-3-yl)-4- azaspiro[2.4] heptane-4- carbonyl)octahydro- 1H-pyrrolo[1,2- a]azepin-6- yl)carbamoyl)benzo [b]thiophen-5- yl)methyl) phosphonic acid | 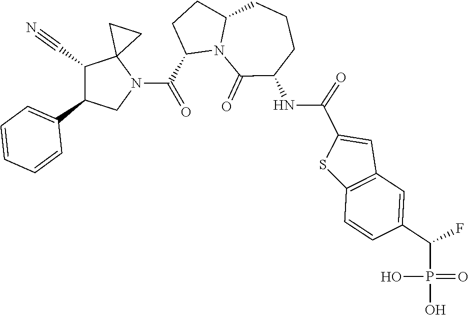 [SMILES: N#C[C@H]1[C@H](c2ccccc2)CN(C(=O)[C@@H]2CC[C@@H]3CCC[C@H](NC(=O)c4cc5cc([C@H](F)P(=O)(O)O)ccc5s4)C(=O)N32)C12CC2] | 641.2 | ¹H NMR (400 MHz, DMSO) δ 8.76 (d, J = 7.5 Hz, 1H), 8.58 (s, 1H), 8.44 (d, J = 4.4 Hz, 1H), 8.24 (s, 1H), 8.04-7.93 (m, 2H), 7.85-7.76 (m, 1H), 7.51 (d, J = 8.8 Hz, 1H), 7.39-7.31 (m, 1H), 5.88-5.67 (m, 1H), 4.65-4.59 (m, 2H), 4.12-4.07 (m, 1H), 3.98-3.93 (m, 1H), 3.75-3.70 (m, 1H), 3.61-3.56 (m, 1H), 2.24-1.55 (m, 14H), 0.57-0.35 (m, 2H) |
| 918 | ((2-(((3S,6S,9aS)-3- (3-(2-cyanopyridin- 4-yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 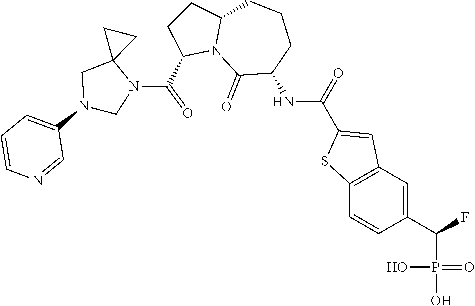 | 626.0 | ¹H NMR (400 MHz, CD₃OD) δ 8.66 (dd, J = 15.7, 5.1 Hz, 1H), 8.14-8.06 (m, 2H), 8.05 (br. s., 1H), 7.98- 7.93 (m, 1H), 7.80- 7.68 (m, 1H), 7.60 (d, J = 8.6 Hz, 1H), 5.91- 5.74 (m, 1H), 4.99- 4.90 (m, 1H), 4.78- 4.68 (m, 1H), 4.64- 4.47 (m, 2H), 4.46- 4.29 (m, 1H), 4.13- 3.97 (m, 3H), 2.39- 2.25 (m, 1H), 2.20- 2.05 (m, 2H), 2.03- 1.77 (m, 7H) |
| 919 | ((R)-(2- (((3S,6S,9aS)-3- ((6S,7R)-7-cyano-6- phenyl-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid | 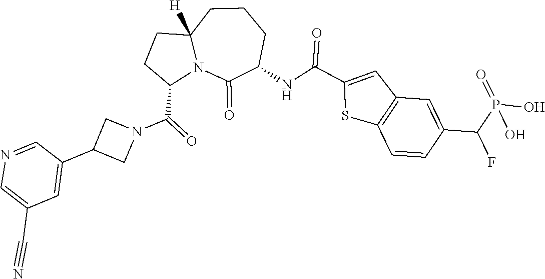 [SMILES: N#Cc1cncc(C2CN(C(=O)[C@@H]3CC[C@@H]4CCC[C@H](NC(=O)c5cc6cc(C(F)P(=O)(O)O)ccc6s5)C(=O)N43)C2)c1] | 665.1 | ¹H NMR (400 MHz, CD₃OD) δ 8.08 (s, 1H), 8.06-8.02 (m, 1H), 7.95 (d, J = 8.6 Hz, 1H), 7.59 (d, J = 8.6 Hz, 1H), 7.48- 7.37 (m, 4H), 7.36- 7.30 (m, 1H), 5.82 (dd, J = 44.7, 7.6 Hz, 1H), 4.77-4.69 (m, 2H), 4.49-4.42 (m, 1H), 4.08-3.97 (m, 1H), 3.89-3.71 (m, 3H), 2.39-2.23 (m, 2H), 2.17-1.76 (m, 10H), 1.04-0.96 (m, 1H), 0.79-0.71 (m, 1H) |
| 920 | ((S)-(2- (((3S,6S,9aS)-3- ((6S,7R)-7-cyano-6- phenyl-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid | 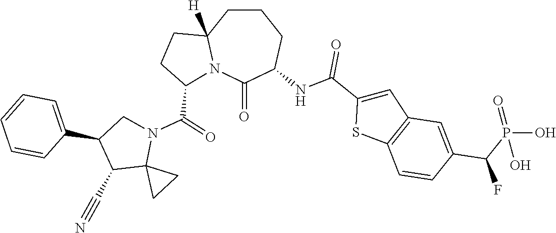 [SMILES: N#C[C@@H]1[C@@H](c2ccccc2)CN(C(=O)[C@@H]2CC[C@@H]3CCC[C@H](NC(=O)c4cc5cc([C@H](F)P(=O)(O)O)ccc5s4)C(=O)N32)C12CC2] | 665.1 | ¹H NMR (400 MHz, CD₃OD) δ 8.08 (s, 1H), 8.05-8.02 (m, 1H), 7.95 (d, J = 8.3 Hz, 1H), 7.59 (d, J = 8.3 Hz, 1H), 7.48- 7.37 (m, 4H), 7.36- 7.30 (m, 1H), 5.82 (dd, J = 44.7, 8.1 Hz, 1H), 4.78-4.68 (m, 2H), 4.50-4.39 (m, 1H), 4.08-3.96 (m, 1H), 3.89-3.71 (m, 3H), 2.38-2.22 (m, |
| | | | | 2H), 2.18-1.73 (m, |
| | | | | 10H), 1.07-0.96 (m, |
| | | | | 1H), 0.80-0.69 (m, |
| | | | | 1H) |
| 921 | ((S)-(2- (((3S,6S,9aS)-3- ((6R,7S)-7-cyano-6- phenyl-4- azaspiro[2.4] heptane-4- carbonyl)-5- oxooctahydro-1H- pyrrolo[1,2- a]azepin-6- yl)carbamoyl)benzo [b]thiophen-5- yl)fluoromethyl) phosphonic acid | 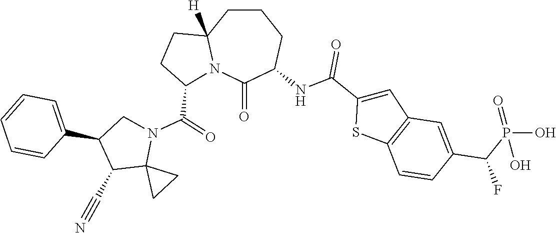 [SMILES: N#C[C@@H]1[C@@H](c2ccccc2)CN(C(=O)[C@@H]2CC[C@@H]3CCC[C@H](NC(=O)c4cc5cc([C@@H](F)P(=O)(O)O)ccc5s4)C(=O)N32)C12CC2] | 665.1 | ¹H NMR (400 MHz, CD₃OD) δ 8.10-8.01 (m, 2H), 7.97-7.91 (m, 1H), 7.63-7.57 (m, 1H), 7.51-7.45 (m, 2H), 7.43-7.28 (m, 3H), 5.89-5.71 (m, 1H), 4.76-4.64 (m, 2H), 4.19-4.11 (m, 1H), 4.10-4.00 (m, 2H), 3.84-3.68 (m, 2H), 2.38-2.24 (m, 2H), 2.24-2.13 (m, 1H), 2.12-1.99 |
| | | | | (m, 2H), 1.98-1.89 |
| | | | | (m, 2H), 1.89-1.77 |
| | | | | (m, 4H), 1.41-1.33 |
| | | | | (m, 1H), 1.04-0.94 |
| | | | | (m, 1H), 0.76-0.64 |
| | | | | (m, 1H). |
| 922 | ((2-(((3S,6S,9aS)-3- ((6R,7S)-7-cyano-6- phenyl-4- azaspiro[2.4] heptane-4- carbonyl)-5- oxooctahydro-1H- pyrrolo[1,2- a]azepin-6- yl)carbamoyl)benzo [b]thiophen-5- yl)methyl) phosphonic acid | 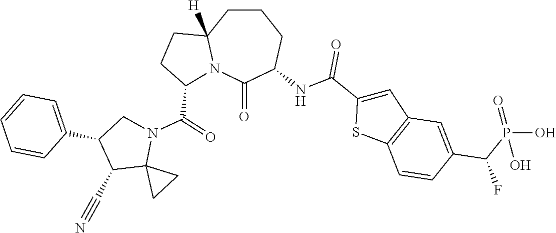 [SMILES: N#C[C@@H]1[C@H](c2ccccc2)CN(C(=O)[C@@H]2CC[C@@H]3CCC[C@H](NC(=O)c4cc5cc([C@@H](F)P(=O)(O)O)ccc5s4)C(=O)N32)C12CC2] | 647.1 | ¹H NMR (400 MHz, CD₃OD) δ 8.01 (s, 1H), 7.90-7.80 (m, 2H), 7.53-7.28 (m, 6H), 4.76-4.64 (m, 2H), 4.19-4.11 (m, 1H), 4.10-3.99 (m, 2H), 3.85-3.69 (m, 2H), 3.29-3.20 (m, 2H), 2.39-2.24 (m, 2H), 2.24-2.13 (m, 1H), 2.12-1.75 (m, 9H), 1.05-0.94 (m, 1H), 0.76-0.64 (m, 1H). |
| 923 | ((S)-(2- (((3S,6S,10aS)-3- ((6S,7R)-7-cyano-6- phenyl-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 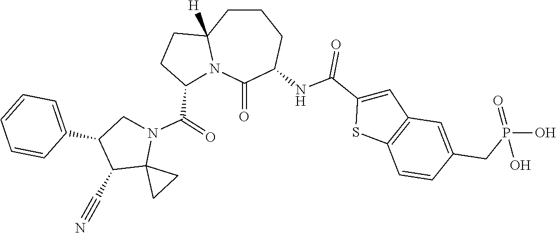 [SMILES: N#C[C@@H]1[C@H](c2ccccc2)CN(C(=O)[C@@H]2CC[C@@H]3CCC[C@H](NC(=O)c4cc5cc(CP(=O)(O)O)ccc5s4)C(=O)N32)C12CC2] | 679.1 | ¹H NMR (400 MHz, CD₃OD) δ 8.10 (s, 1H), 8.04-7.97 (m, 1H), 7.92 (d, J = 8.3 Hz, 1H), 7.58 (d, J = 8.6 Hz, 1H), 7.44- 7.28 (m, 5H), 5.81 (dd, J = 44.7, 7.3 Hz, 1H), 5.07-4.97 (m, 1H), 4.68-4.52 (m, 2H), 4.45-4.32 (m, 1H), 3.86-3.69 (m, 3H), 2.37-2.13 (m, 3H), 2.05-1.78 (m, |
| | | | | 9H), 1.69-1.54 (m, |
| | | | | 2H), 1.04-0.94 (m, |
| | | | | 1H), 0.77-0.68 (m, |
| | | | | 1H) |
| 924 | ((R)-(2- (((3S,6S,10aS)-3- ((6R,7S)-7-cyano-6- phenyl-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 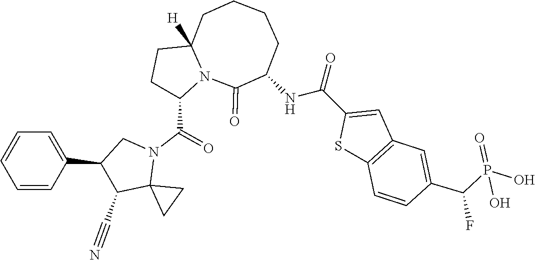 [SMILES: N#C[C@@H]1[C@@H](c2ccccc2)CN(C(=O)[C@@H]2CC[C@@H]3CCCC[C@H](NC(=O)c4cc5cc([C@@H](F)P(=O)(O)O)ccc5s4)C(=O)N32)C12CC2] | 679.1 | ¹H NMR (400 MHz, CD₃OD) δ 8.10 (s, 1H), 8.02 (s, 1H), 7.96-7.88 (m, 1H), 7.62-7.56 (m, 1H), 7.54-7.26 (m, 5H), 5.86 (d, J = 5.9 Hz, 0.5H), 5.75 (d, J = 5.9 Hz, 0.5H), 5.10-4.95 (m, 1H), 4.64-4.54 (m, 1H), 4.47-4.35 (m, 1H), 4.22-4.07 (m, 2H), 3.85-3.65 (m, 2H), 2.43-2.29 (m, 2H), 2.28-2.15 (m, 1H), 2.12-1.89 |
| | | | | (m, 6H), 1.87-1.74 |
| | | | | (m, 3H), 1.72-1.55 |
| | | | | (m, 2H), 1.05-0.91 |
| | | | | (m, 1H), 0.76-0.61 |
| | | | | (m, 1H). |
| 925 | (fluoro(2- (((3S,6S,9aS)-3-(3- (2-fluoro-3- methoxypyridin-4- yl)azetidine-1- carbonyl)-5- oxooctahydro-1H- pyrrolo[1,2- a]azepin-6- yl)carbamoyl)benzo [b]thiophen-5- yl)methyl) phosphonic acid | 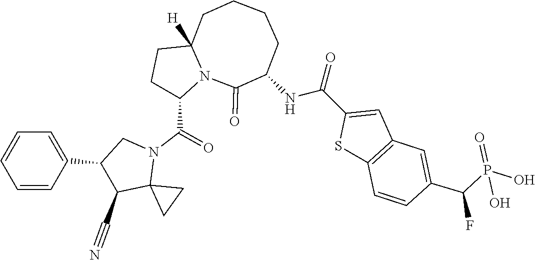 [SMILES: N#C[C@H]1[C@H](c2ccccc2)CN(C(=O)[C@@H]2CC[C@@H]3CCCC[C@H](NC(=O)c4cc5cc([C@H](F)P(=O)(O)O)ccc5s4)C(=O)N32)C12CC2] | 649.0 | ¹H NMR (400 MHz, CD₃OD) δ 8.06 (d, J = 10.5 Hz, 1H), 7.88- 7.85 (m, 1H), 7.70- 7.61 (m, 1H), 7.44 (d, J = 5.4 Hz, 1H), 7.33 (d, J = 5.1 Hz, 1H), 5.74-5.55 (m, 1H), 4.95-4.92 (m, 1H), 4.77-4.71 (m, 1H), 4.67 (s, 1H), 4.61- 4.56 (m, 1H), 4.51- 4.45 (m, 1H), 4.42- 4.31 (m, 1H), 4.30- 4.22 (m, 1H), 4.13- 4.04 (m, 2H), 3.96 (d, |
| | | | | J = 2.7 Hz, 3H), 2.36- |
| | | | | 2.26 (m, 1H), 2.18- |
| | | | | 1.79 (m, 9H) |
| 926 | ((S)-fluoro(2- (((3S,6S,9aS)-3-(3- (4-((S)-2- (methoxymethyl) azetidin-1-yl) pyridin-3- yl)azetidine-1- carbonyl)-5- oxooctahydro-1H- pyrrolo[1,2- a]azepin-6- yl)carbamoyl)benzo [b]thiophen-5- yl)methyl) phosphonic acid | 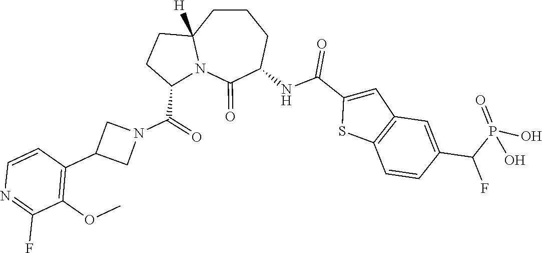 [SMILES: COc1c(C2CN(C(=O)[C@@H]3CC[C@@H]4CCC[C@H](NC(=O)c5cc6cc(C(F)P(=O)(O)O)ccc6s5)C(=O)N43)C2)ccnc1F] | 700.1 | ¹H NMR (400 MHz, CD₃OD) δ 8.27 (s, 1H), 8.11-8.03 (m, 2H), 7.84-7.78 (m, 1H), 7.75-7.68 (m, 1H), 6.76 (dd, J = 5.7, 4.0 Hz, 1H), 5.68- 5.54 (m, 1H), 4.76- 4.71 (m, 1H), 4.67- 4.55 (m, 2H), 4.52- 4.44 (m, 2H), 4.34- 4.13 (m, 3H), 4.10- 4.02 (m, 2H), 3.74- 3.66 (m, 1H), 3.59 (t, J = 4.6 Hz, 2H), 3.36- |
| | | | | 3.33 (m, 3H), 2.42- |
| | | | | 2.16 (m, 4H), 2.16- |
| | | | | 1.76 (m, 9H) |
| 927 | (fluoro(2- (((3S,6S,9aS)-3-(3- (2-hydroxypyrimidin- 4-yl)azetidine-1- carbonyl)-5- oxooctahydro-1H- pyrrolo[1,2- a]azepin-6- yl)carbamoyl)benzo [b]thiophen-5- yl)methyl) phosphonic acid | 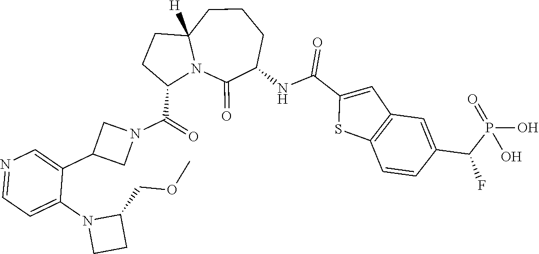 [SMILES: COC[C@@H]1CCN1c1ccncc1C1CN(C(=O)[C@@H]2CC[C@@H]3CCC[C@H](NC(=O)c4cc5cc([C@@H](F)P(=O)(O)O)ccc5s4)C(=O)N32)C1] | 618.1 | ¹H NMR (400 MHz, CD₃OD) δ 8.17-7.87 (m, 4H), 7.72-7.54 (m, 1H), 6.87-6.51 (m, 1H), 5.87-5.61 (m, 1H), 4.70-4.61 (m, 0.5H), 4.60-4.51 (m, 1H), 4.50-4.30 (m, 1H), 4.27-4.19 (m, 0.5H), 4.17-4.04 (m, 1.5H), 3.77-3.64 (m, 0.5H), 3.24-3.11 (m, 1H), 2.77-2.65 (m, 1H), 2.40-2.26 (m, 1H), 2.21-2.05 (m, 1H), 2.04-1.91 |
| | | | | (m, 1H), 1.88-1.78 |
| | | | | (m, 2H), 1.62-1.41 |
| | | | | (m, 1H), 1.41-1.34 |
| | | | | (m, 4H), 1.32-1.17 |
| | | | | (m, 2H) |
| 928 | ((S)-(2- (((3S,6S,9aS)-3-(3- (4-ethoxy-5- fluoropyridin-3- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 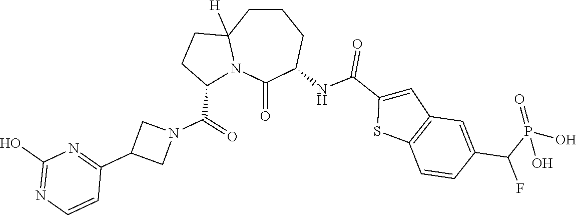 [SMILES: O=C(N[C@H]1CCCC2CC[C@@H](C(=O)N3CC(c4ccnc(O)n4)C3)N2C1=O)c1cc2cc(C(F)P(=O)(O)O)ccc2s1] | 663.1 | ¹H NMR (400 MHz, CD₃OD) δ 8.68-8.53 (m, 1H), 8.17-8.04 (m, 3H), 7.89-7.82 (m, 1H), 7.64 (d, J = 8.8 Hz, 1H), 5.75- 5.54 (m, 1H), 4.77- 4.72 (m, 1H), 4.66- 4.45 (m, 3H), 4.42- 4.21 (m, 1H), 4.13- 3.84 (m, 5H), 2.35- 2.25 (m, 1H), 2.13- 1.80 (m, 9H), 1.45 (t, |
| | | | | J = 7.2 Hz, 1H), 1.25 |
| | | | | (t, J = 7.2 Hz, 2H) |
| 929 | ((2-(((3S,6S,10aS)- 3-((6R,7S)-7-cyano- 6-phenyl-4- azaspiro[2.4] heptane-4- carbonyl)-5- oxodecahydropyrrolo [1,2-a]azocin-6- yl)carbamoyl)benzo [b]thiophen-5- yl)methyl) phosphonic acid | 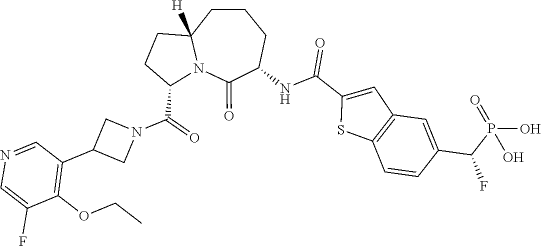 [SMILES: CCOc1c(F)cncc1C1CN(C(=O)[C@@H]2CC[C@@H]3CCC[C@H](NC(=O)c4cc5cc([C@@H](F)P(=O)(O)O)ccc5s4)C(=O)N32)C1] | 661.4 | ¹H NMR (400 MHz, CD₃OD) δ 8.03 (s, 1H), 7.89-7.76 (m, 2H), 7.53-7.25 (m, 6H), 5.05-4.95 (m, 1H), 4.62-4.54 (m, 1H), 4.45-4.34 (m, 1H), 4.21-4.04 (m, 2H), 3.82-3.72 (m, 1H), 3.71-3.64 (m, 1H), 3.28-3.18 (m, 2H), 2.44-2.29 (m, 2H), 2.27-2.14 (m, 1H), 2.11-1.88 (m, 6H), 1.87-1.73 (m, |
| | | | | 3H), 1.70-1.55 (m, |
| | | | | 2H), 1.02-0.92 (m, |
| | | | | 1H), 0.75-0.61 (m, |
| | | | | 1H) |
| 930 | ((R)-(2- (((3S,6S,9R,10aR)- 3-(3-(4-(azetidin-1- yl)pyridin-3- yl)azetidine-1- carbonyl)-5-oxo-9- propyldecahydro- pyrrolo[1,2-a] azocin-6- yl)carbamoyl)benzo [b]thiophen-5- yl)fluoromethyl) phosphonic acid | 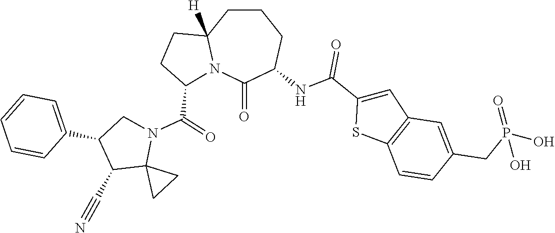 [SMILES: N#C[C@@H]1[C@H](c2ccccc2)CN(C(=O)[C@@H]2CC[C@@H]3CCC[C@H](NC(=O)c4cc5cc(CP(=O)(O)O)ccc5s4)C(=O)N32)C12CC2] | 712.1 | ¹H NMR (400 MHz, CD₃OD) δ 8.14-7.48 (m, 6H), 6.34-6.05 (m, 1H), 5.78-5.52 (m, 1H), 5.04-5.03 (m, 1H), 4.79-4.41 (m, 3H), 4.38-4.14 (m, 5H), 4.11-3.96 (m, 2H), 2.49-2.30 (m, 2H), 2.28-2.15 (m, 2H), 2.09-1.79 (m, 6H), 1.70-1.38 (m, 5H), 1.34-1.27 (m, 3H), 0.98-0.89 (m, 3H) |
| 931 | ((R)-(2- (((3S,6S,9R,10aR)- 3-(3-(4-(azetidin-1- yl)pyridin-3- yl)azetidine-1- carbonyl)-9-ethyl-5- oxodecahydropyrrolo [1,2-a]azocin-6- yl)carbamoyl)benzo [b]thiophen-5- yl)fluoromethyl) phosphonic acid | 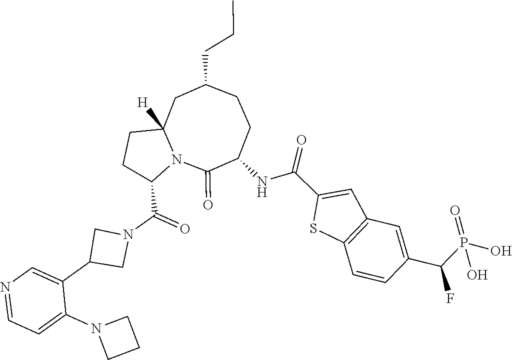 [SMILES: CCC[C@@H]1CC[C@H](NC(=O)c2cc3cc([C@H](F)P(=O)(O)O)ccc3s2)C(=O)N2[C@H](CC[C@H]2C(=O)N2CC(c3cnccc3N3CCC3)C2)C1] | 698.1 | ¹H NMR (400 MHz, CD₃OD) δ 8.10-7.39 (m, 6H), 6.26-6.01 (m, 1H), 5.76-5.54 (m, 1H), 5.07-4.96 (m, 1H), 4.77-4.42 (m, 3H), 4.34-4.13 (m, 5H), 4.11-3.98 (m, 2H), 2.47-2.16 (m, 4H), 2.10-1.79 (m, 6H), 1.67-1.49 (m, 3H), 1.45-1.37 (m, 2H), 1.34-1.28 (m, 1H), 0.99-0.94 (m, 3H) |
| 932 | ((2-(((3S,6S,9aS)-3- (3-(2-cyano-3- methoxypyridin-4- yl)azetidine-1- carbonyl)-5- oxooctahydro-1H- pyrrolo[1,2- a]azepin-6- yl)carbamoyl)benzo [b]thiophen-5- yl)fluoromethyl) phosphonic acid | 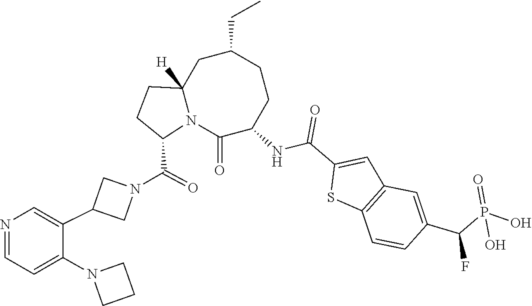 | 656.1 | ¹H NMR (400 MHz, CD₃OD) δ 8.49-8.33 (m, 1H), 8.10-8.00 (m, 2H), 7.98-7.90 (m, 1H), 7.85-7.67 (m, 1H), 7.59 (d, J = 8.3 Hz, 1H), 5.94- 5.70 (m, 1H), 4.96- 4.89 (m, 1H), 4.78- 4.71 (m, 1H), 4.71- 4.65 (m, 1H), 4.62- 4.51 (m, 2H), 4.51- 4.31 (m, 1H), 4.30- 4.19 (m, 1H), 4.10 (d, J = 1.7 Hz, 3H), 4.08- 4.03 (m, 1H), 2.38- 2.25 (m, 1H), 2.20- 2.05 (m, 2H), 2.04- 1.75 (m, 7H) |
| 933 | ((2- (((3S,6S,8R,9aR)-3- (3-(4-(3- methoxyazetidin-1- yl)pyridin-3- yl)azetidine-1- carbonyl)-8-methyl- 5-oxooctahydro-1H- pyrrolo[1,2- a]azepin-6- yl)carbamoyl)benzo [b]thiophen-5- yl)methyl) phosphonic acid | 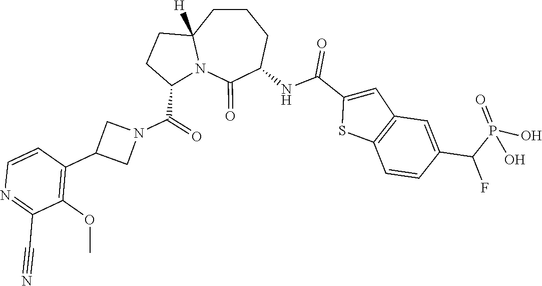 [SMILES: COc1c(C2CN(C(=O)[C@@H]3CC[C@@H]4CCC[C@H](NC(=O)c5cc6cc(C(F)P(=O)(O)O)ccc6s5)C(=O)N43)C2)ccnc1C#N] | 682.4 | ¹H NMR (400 MHz, CD₃OD) δ 7.93-7.83 (m, 1H), 7.81-7.67 (m, 3H), 7.53-7.39 (m, 2H), 6.33-6.14 (m, 1H), 4.80-4.67 (m, 2H), 4.63-4.44 (m, 3H), 4.43-4.25 (m, 2H), 4.22-4.15 (m, 1H), 4.14-4.01 (m, 3H), 3.54-3.41 (m, 1H), 3.39-3.34 (m, 2H), 3.16-3.02 (m, 2H), 2.40-2.24 (m, 1H), 2.17-2.00 (m, 3H), 1.99-1.88 (m, 2H), 1.84-1.72 (m, 1H), 1.70-1.45 |
| | | | | (m, 2H), 1.38-1.25 |
| | | | | (m, 2H), 1.05 (t, J = |
| | | | | 6.8 Hz, 3H) |
| 934 | ((R)-(2- (((3S,6S,9S,10aR)- 9-(azetidin-1-yl)-3- ((6S,7R)-7-cyano-6- phenyl-4- azaspiro[2.4] heptane- 4-carbonyl)-5- oxodecahydropyrrolo [1,2-a]azocin-6- yl)carbamoyl)benzo [b]thiophen-5- yl)fluoromethyl) phosphonic acid | 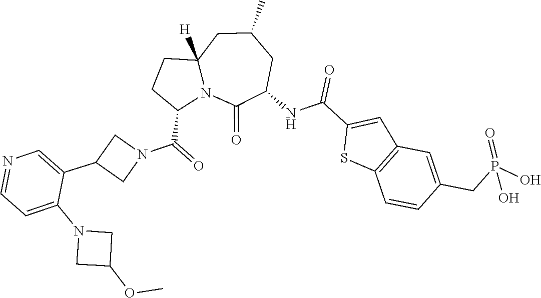 [SMILES: COC1CN(c2ccncc2C2CN(C(=O)[C@@H]3CC[C@@H]4C[C@H](C)C[C@H](NC(=O)c5cc6cc(CP(=O)(O)O)ccc6s5)C(=O)N43)C2)C1] | 734.1 | ¹H NMR (400 MHz, CD₃OD) δ 8.09 (s, 1H), 7.98-7.92 (m, 2H), 7.78-7.76 (m, 1H), 7.41-7.26 (m, 5H), 5.81-5.62 (m, 1H), 4.55-4.37 (m, 2H), 3.99-3.76 (m, 4H), 3.71-3.51 (m, 3H), 3.13-3.03 (m, 1H), 2.36-2.03 (m, 5H), 1.88-1.67 (m, 4H), 1.65-1.49 (m, 2H), 1.43-1.37 (m, 1H), 1.34-1.29 (m, 2H), 1.00-0.87 (m, 1H), 0.73-0.51 (m, 2H), 0.36-0.22 (m, 1H) |
| 935 | ((S)-(2- (((3S,6S,9S,10aR)- 9-(azetidin-1-yl)-3- ((6S,7R)-7-cyano-6- phenyl-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 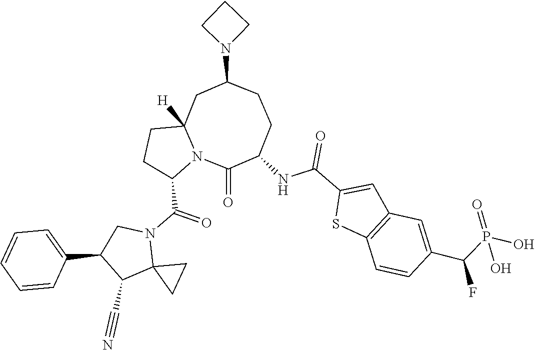 [SMILES: N#C[C@@H]1[C@@H](c2ccccc2)CN(C(=O)[C@@H]2CC[C@@H]3C[C@@H](N4CCC4)CC[C@H](NC(=O)c4cc5cc([C@H](F)P(=O)(O)O)ccc5s4)C(=O)N32)C12CC2] | 734.1 | ¹H NMR (400 MHz, CD₃OD) δ 8.19-8.10 (m, 2H), 7.92-7.90 (m, 1H), 7.52-7.50 (m, 1H), 7.41-7.26 (m, 5H), 5.79-5.60 (m, 1H), 4.64-4.47 (m, 1H), 4.44-4.26 (m, 1H), 4.05-3.78 (m, 4H), 3.72-3.53 (m, 3H), 3.15-3.08 (m, 1H), 2.36-2.20 (m, 4H), 2.13-2.00 (m, 1H), 1.85-1.68 (m, 4H), 1.67-1.49 (m, 3H), 1.35-1.21 (m, 2H), 0.95-0.90 (m, 1H), 0.73-0.64 (m, 1H), 0.52-0.48 (m, 1H), 0.40-0.28 (m, 1H) |
| 936 | ((R)-(2- (((3S,6S,9S,10aR)- 9-(azetidin-1-yl)-3- ((6R,7S)-7-cyano-6- phenyl-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 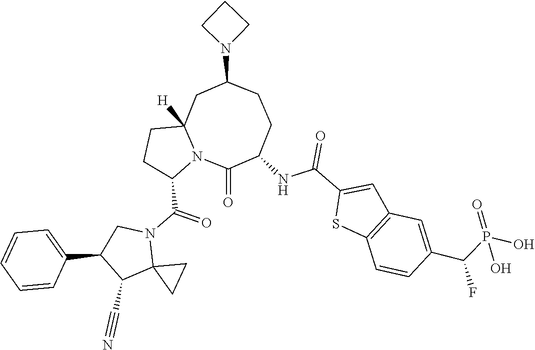 [SMILES: N#C[C@@H]1[C@@H](c2ccccc2)CN(C(=O)[C@@H]2CC[C@@H]3C[C@@H](N4CCC4)CC[C@H](NC(=O)c4cc5cc([C@@H](F)P(=O)(O)O)ccc5s4)C(=O)N32)C12CC2] | 734.1 | ¹H NMR (400 MHz, CD₃OD) δ 8.19-8.10 (m, 1H), 8.00-7.94 (m, 2H), 7.80-7.74 (m, 1H), 7.44-7.24 (m, 5H), 5.80-5.61 (m, 1H), 4.47 (t, J = 8.4 Hz, 1H), 4.11- 3.96 (m, 3H), 3.88- 3.78 (m, 1H), 3.76- 3.60 (m, 4H), 3.18- 3.12 (m, 1H), 2.43- 2.25 (m, 4H), 2.16- 2.06 (m, 1H), 1.88- 1.82 (m, 1H), 1.79- 1.69 (m, 3H), 1.65- 1.55 (m, 2H), 1.46- 1.38 (m, 1H), 1.35- 1.28 (m, 2H), 0.97- 0.90 (m, 1H), 0.72- 0.53 (m, 2H), 0.43- 0.33 (m, 1H) |
| 937 | ((S)-(2- (((3S,6S,9S,10aR)- 9-(azetidin-1-yl)-3- ((6R,7S)-7-cyano-6- phenyl-4- azaspiro[2.4] heptane-4- carbonyl)-5- oxodecahydropyrrolo [1,2-a]azocin-6- yl)carbamoyl)benzo [b]thiophen-5- yl)fluoromethyl) phosphonic acid | 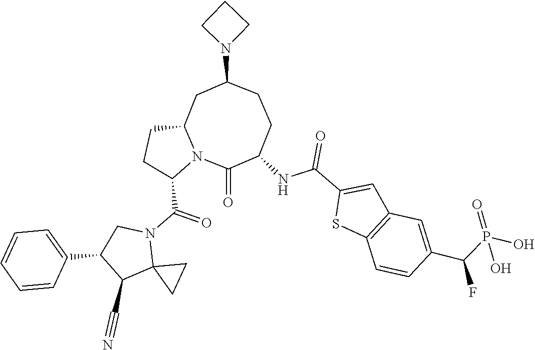 [SMILES: N#C[C@H]1[C@H](c2ccccc2)CN(C(=O)[C@@H]2CC[C@@H]3C[C@@H](N4CCC4)CC[C@H](NC(=O)c4cc5cc([C@H](F)P(=O)(O)O)ccc5s4)C(=O)N32)C12CC2] | 734.1 | ¹H NMR (400 MHz, CD₃OD) δ 8.22-8.11 (m, 2H), 7.92-7.90 (m, 1H), 7.53-7.51 (m, 1H), 7.45-7.24 (m, 5H), 5.79-5.58 (m, 1H), 4.48-4.43 (m, 1H), 4.11-3.96 (m, 3H), 3.85-3.75 (m, 1H), 3.75-3.52 (m, 4H), 3.14-3.04 (m, 1H), 2.41-2.18 (m, 4H), 2.15-2.03 (m, 1H), 1.88-1.53 (m, 6H), 1.47-1.43 (m, 1H), 1.35-1.28 (m, 2H), 0.96-0.91 (m, 1H), 0.69-0.48 (m, 2H), 0.42-0.32 (m, 1H) |
| 938 | ((R)-(2- (((3S,6S,9S,10aR)- 3-(3-(4-(azetidin-1- yl)pyridin-3- yl)azetidine-1- carbonyl)-9-ethyl-5- oxodecahydropyrrolo [1,2-a]azocin-6- yl)carbamoyl)benzo [b]thiophen-5- yl)fluoromethyl) phosphonic acid | 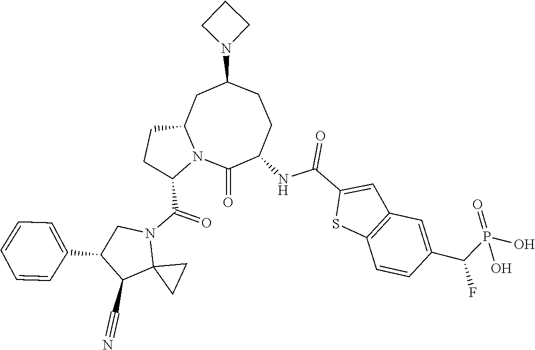 [SMILES: N#C[C@H]1[C@H](c2ccccc2)CN(C(=O)[C@@H]2CC[C@@H]3C[C@@H](N4CCC4)CC[C@H](NC(=O)c4cc5cc([C@@H](F)P(=O)(O)O)ccc5s4)C(=O)N32)C12CC2] | 698.1 | ¹H NMR (400 MHz, CD₃OD) δ 8.55-8.23 (m, 1H), 8.10-7.98 (m, 3H), 7.83-7.68 (m, 2H), 6.41-6.35 (m, 1H), 5.71-5.50 (m, 1H), 4.94-4.78 (m, 1H), 4.72-4.58 (m, 1H), 4.52-4.39 (m, 2H), 4.36-4.26 (m, 1H), 4.18-3.98 (m, 6H), 2.42-1.88 (m, 7H), 1.87-1.45 (m, 5H), 1.44-1.27 (m, 3H), 1.07-1.03 (m, 1H), 0.98-0.89 (m, 3H) |
| 939 | (fluoro(2- (((3S,6S,9aS)-3-(3- (4-isopropylpyridin- 3-yl)azetidine-1- carbonyl)-5- oxooctahydro-1H- pyrrolo[1,2- a]azepin-6- yl)carbamoyl)benzo [b]thiophen-5- yl)methyl) phosphonic acid | 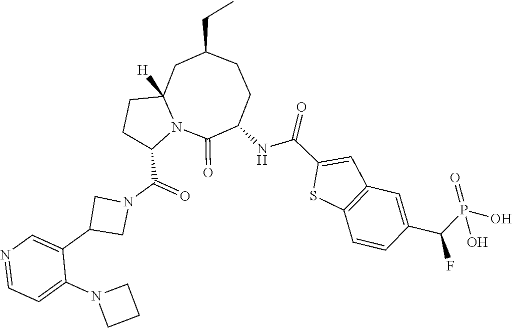 [SMILES: CC[C@H]1CC[C@H](NC(=O)c2cc3cc([C@H](F)P(=O)(O)O)ccc3s2)C(=O)N2[C@H](CC[C@H]2C(=O)N2CC(c3cnccc3N3CCC3)C2)C1] | 643.1 | ¹H NMR (400 MHz, CD₃OD) δ 8.52-8.42 (m, 1H), 8.25-8.13 (m, 1H), 8.01-7.92 (m, 1H), 7.88-7.79 (m, 1H), 7.79-7.71 (m, 1H), 7.64-7.46 (m, 2H), 5.83-5.59 (m, 1H), 4.95-4.90 (m, 1H), 4.78-4.67 (m, 1H), 4.64-4.50 (m, 2H), 4.46-4.36 (m, 1H), 4.33-4.17 (m, 1H), 4.15-3.97 (m, 2H), 3.09-2.93 (m, 1H), 2.39-2.26 (m, 1H), 2.22-2.07 (m, 2H), 2.07-1.79 (m, 7H), 1.26-1.13 (m, 6H) |

### Compounds Prepared According to General Scheme 2: Phosphonate Esters

The following compounds in Table 110 were prepared according to the representative procedure described above for the synthesis of S,S′-(((((2-(((3S,6S,10aS)-3-((3 S,4R)-3-cyano-4-phenylpyrrolidine-1l-carbonyl)-5-oxodecahydropyrrolo[1,2-a]azocin-6-yl)carbamoyl)benzo[b]thiophen-5-yl)methyl)phosphoryl)bis(oxy))bis(ethane-2,1-diyl))bis(3-hydroxy-2,2-dimethylpropanethioate)trifluoroacetate (199) utilizing the appropriate starting materials and modifications.

TABLE 110

| Com- | | |
|---|---|---|
| pound | Name | Structure |
| 940 | S,S′-((((difluoro(2- (((3S,6S,9aS)-5- oxo-3-(3- (pyrimidin-2- yl)azetidine-1- carbonyl)octahydro- 1H-pyrrolo[1,2- a]azepin-6- yl)carbamoyl)benzo [b]thiophen-5- yl)methyl) phosphoryl) bis(oxy))bis(ethane- 2,1-diyl)) dibutanethioate | 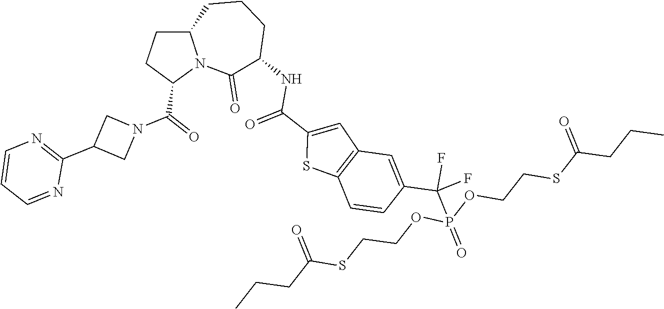 [SMILES: CCCC(=O)SCCOP(=O)(OCCSC(=O)CCC)C(F)(F)c1ccc2sc(C(=O)N[C@H]3CCC[C@H]4CC[C@@H](C(=O)N5CC(c6ncccn6)C5)N4C3=O)cc2c1] |
| 941 | propyl ((R)-((R)-(2- (((3S,6S, 9aS)-3-(3-(4- (dimethylamino) pyridin-3-yl) azetidine- 1-carbonyl)-5- oxooctahydro-1H- pyrrolo[1,2- a]azepin-6- yl)carbamoyl)benzo [b]thiophen-5- yl)fluoromethyl) (phenoxy) phosphoryl)- L-alaninate | 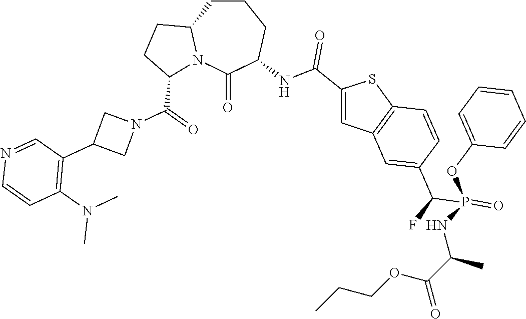 [SMILES: CCCOC(=O)[C@H](C)N[P@](=O)(Oc1ccccc1)[C@@H](F)c1ccc2sc(C(=O)N[C@H]3CCC[C@H]4CC[C@@H](C(=O)N5CC(c6cnccc6N(C)C)C5)N4C3=O)cc2c1] |
| | or | or |
| | propyl ((S)-((R)-(2- (((3S,6S,9aS)-3-(3- (4- (dimethylamino) pyridin-3-yl) azetidine- 1-carbonyl)-5- oxooctahydro-1H- pyrrolo[1,2- a]azepin-6- yl)carbamoyl)benzo [b]thiophen-5- yl)fluoromethyl) (phenoxy) phosphoryl)- L-alaninate | 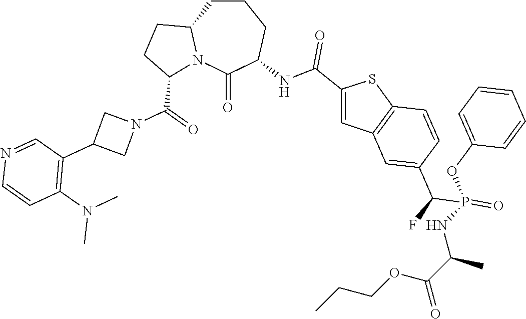 [SMILES: CCCOC(=O)[C@H](C)N[P@@](=O)(Oc1ccccc1)[C@@H](F)c1ccc2sc(C(=O)N[C@H]3CCC[C@H]4CC[C@@H](C(=O)N5CC(c6cnccc6N(C)C)C5)N4C3=O)cc2c1] |
| | | HPLC peak 2 was used for biological testing |
| 942 | propyl (((R)-(2- (((3S,6S, 9aS)-3-(3-(4- (dimethylamino) pyridin-3-yl) azetidine- 1-carbonyl)-5- oxooctahydro-1H- pyrrolo[1,2- a]azepin-6- yl)carbamoyl)benzo [b]thiophen-5- yl)fluoromethyl) (phenoxy) phosphoryl)- L-alaninate | 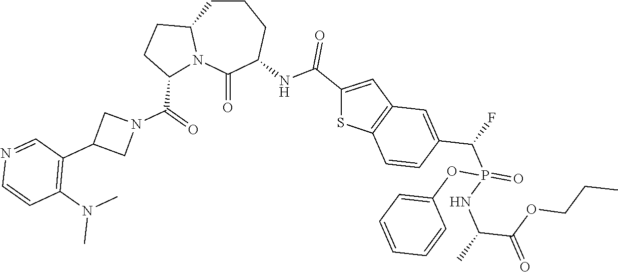 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cnccc6N(C)C)C5)N4C3=O)cc2c1] |
| 943 | propyl ((fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 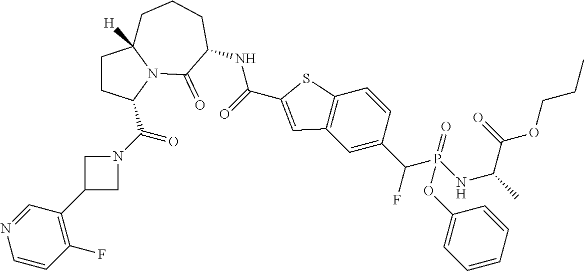 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cnccc6F)C5)N4C3=O)cc2c1] |
| 944 | ethyl (2S)-2-((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino) butanoate | 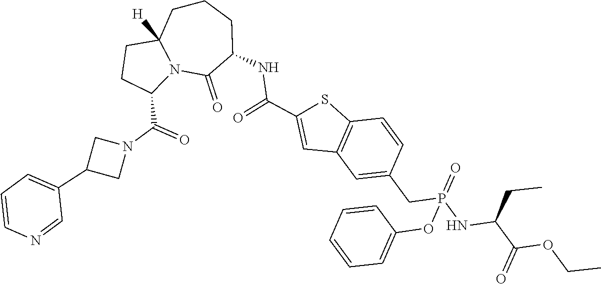 [SMILES: CCOC(=O)[C@H](CC)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] |
| 945 | isobutyl O-methyl- N-(((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- serinate | 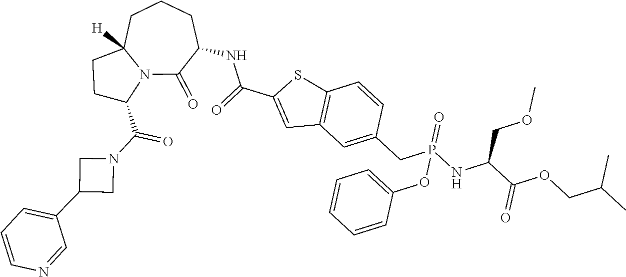 |
| 946 | benzyl O-methyl- N-(((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- serinate | 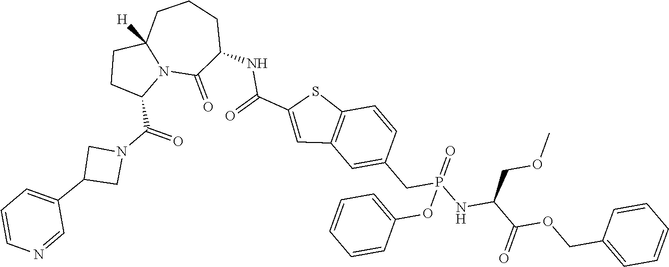 |
| 947 | isobutyl (2S)-2- ((((2-(((3S,6S,9aS)- 5-oxo-3-(3- (pyridin-3- yl)azetidine-1- carbonyl)octahydro- 1H-pyrrolo[1,2- a]azepin-6- yl)carbamoyl)benzo [b]thiophen-5- yl)methyl)(phenoxy) phosphoryl)amino) butanoate | 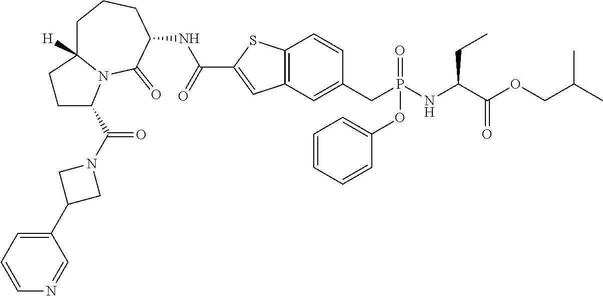 [SMILES: CC[C@H](NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1)C(=O)OCC(C)C] |
| 948 | isobutyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl) (phenoxy) phosphoryl)-L- valinate | 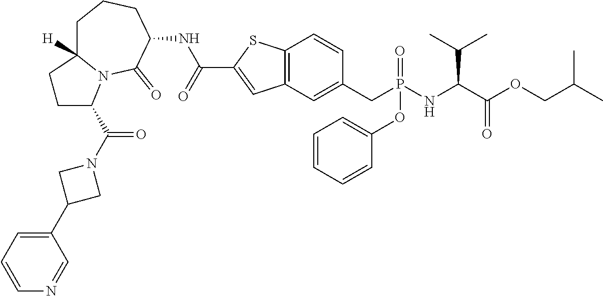 [SMILES: CC(C)COC(=O)[C@@H](NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1)C(C)C] |
| 949 | ethyl (((2- (((3S,6S,9aS)-5- oxo-3-(3-(pyridin- 3-yl)azetidine-1- carbonyl) octahydro- 1H-pyrrolo[1,2- a]azepin-6- yl)carbamoyl)benzo [b]thiophen-5- yl)methyl) (phenoxy) phosphoryl)-L- valinate | 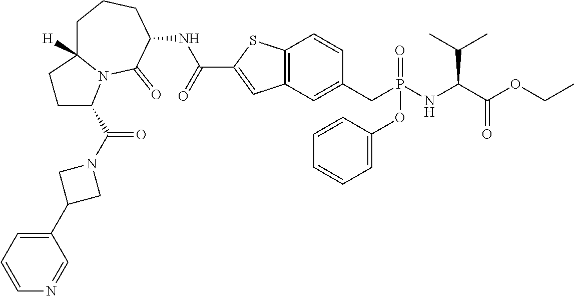 [SMILES: CCOC(=O)[C@@H](NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1)C(C)C] |
| 950 | propyl (((2- (((3S,6S,10aS)-3- (3-(2,2- difluoroethoxy)-3- (pyridin-3- yl)azetidine-1- carbonyl)-5- oxodeca- hydropyrrolo [1,2-a]azocin-6- yl)carbamoyl) benzo [b]thiophen-5- yl)fluoromethyl) (phenoxy) phosphoryl)- L-alaninate | 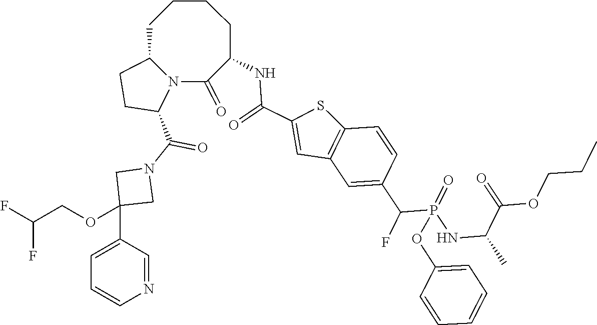 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CC(OCC(F)F)(c6cccnc6)C5)N4C3=O)cc2c1] |
| 951 | propyl (((2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl) (phenoxy) phosphoryl)-L- alaninate | 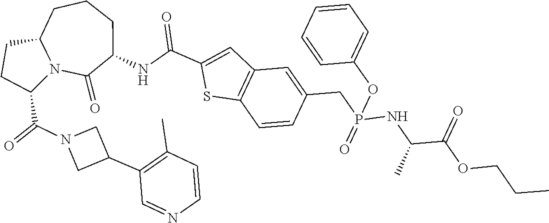 [SMILES: CCCOC(=O)[C@H](C)NP(=O)(Cc1ccc2sc(C(=O)N[C@H]3CCC[C@H]4CC[C@@H](C(=O)N5CC(c6cnccc6C)C5)N4C3=O)cc2c1)Oc1ccccc1] |
| 952 | propyl ((fluoro(2- (((3S,6S,9aS)-5- oxo-3-(1′H- spiro [azetidine-3,3′- furo[3,4- c]pyridine]-1- carbonyl) octahydro- 1H-pyrrolo[1,2- a]azepin-6- yl)carbamoyl) benzo [b]thiophen-5- yl)methyl) (phenoxy) phosphoryl)-L- alaninate | 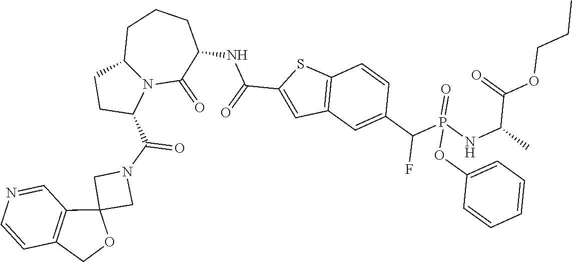 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC6(C5)OCc5ccncc56)N4C3=O)cc2c1] |
| 953 | propyl (((S)-(2- (((3S,6S, 9aS)-3-(3-(4- (dimethylamino) pyridin-3-yl) azetidine- 1-carbonyl)-5- oxooctahydro-1H- pyrrolo[1,2- a]azepin-6- yl)carbamoyl) benzo [b]thiophen-5- yl)fluoromethyl) (phenoxy) phosphoryl)- L-alaninate | 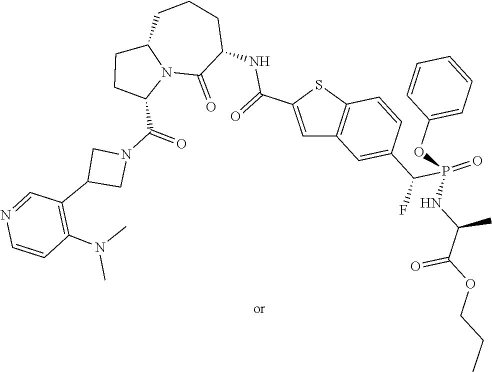 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cnccc6N(C)C)C5)N4C3=O)cc2c1] |
| | or | or |
| | propyl (((S)-(2- (((3S,6S,9aS)- 3-(3-(4- (dimethylamino) pyridin-3- yl)azetidine- 1-carbonyl)-5- oxooctahydro-1H- pyrrolo[1,2- a]azepin-6- yl)carbamoyl) benzo [b]thiophen-5- yl)fluoromethyl) (phenoxy) phosphoryl)- L-alaninate | 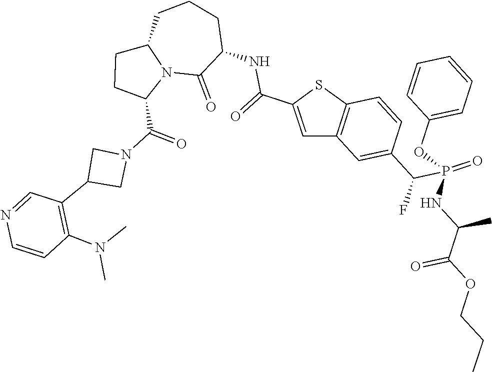 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cnccc6N(C)C)C5)N4C3=O)cc2c1] |
| 954 | propyl (((S)-(2- (((3S,6S,9aS)- 3-(3-(4- (dimethylamino) pyridin-3- yl)azetidine- 1-carbonyl)-5- oxooctahydro-1H- pyrrolo[1,2- a]azepin-6-yl) carbamoyl)benzo [b]thiophen-5- yl)fluoromethyl) (phenoxy) phosphoryl)- L-alaninate or propyl (((S)-(2- (((3S,6S,9aS)-3-(3- (4- (dimethylamino) pyridin-3-yl) azetidine- 1-carbonyl)-5- oxooctahydro-1H- pyrrolo[1,2- a]azepin-6- yl)carbamoyl)benzo | 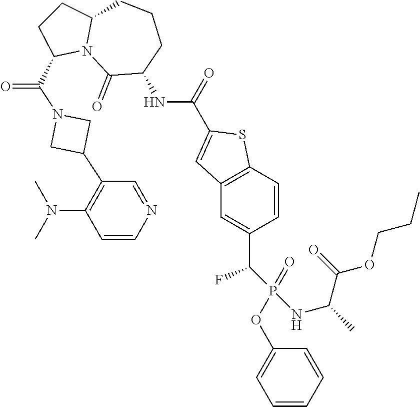 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cnccc6N(C)C)C5)N4C3=O)cc2c1] CFTE-27B, SFC Peak 2 was used for synthesis and biological testing |
| | [b]thiophen-5- | |
| | yl)fluoromethyl) | |
| | (phenoxy) | |
| | phosphoryl)- | |
| | L-alaninate | |
| 955 | isopropyl ((S)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl) (phenoxy) phosphoryl)-L- alaninate | 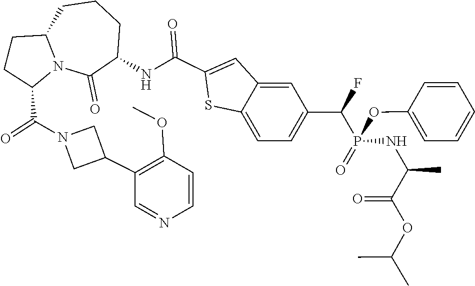 [SMILES: COc1ccncc1C1CN(C(=O)[C@@H]2CC[C@@H]3CCC[C@H](NC(=O)c4cc5cc([C@@H](F)[P@@](=O)(N[C@@H](C)C(=O)OC(C)C)Oc6ccccc6)ccc5s4)C(=O)N32)C1] |
| | or | or |
| | isopropyl ((R)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl) (phenoxy) phosphoryl)-L- alaninate | 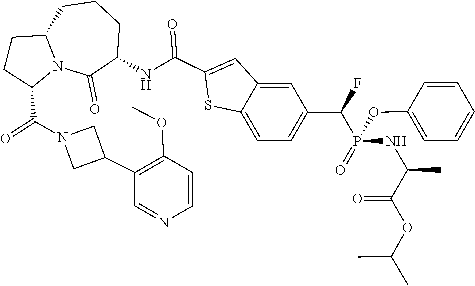 [SMILES: COc1ccncc1C1CN(C(=O)[C@@H]2CC[C@@H]3CCC[C@H](NC(=O)c4cc5cc([C@@H](F)[P@](=O)(N[C@@H](C)C(=O)OC(C)C)Oc6ccccc6)ccc5s4)C(=O)N32)C1] |
| | | Less polar linker building block |
| | | used for synthesis and biological |
| | | testing |
| 956 | isopropyl ((S)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl) (phenoxy) phosphoryl)- L-alaninate or isopropyl ((R)-((S)- fluoro(2- (((3S,6S,9aS)-3-(3- | 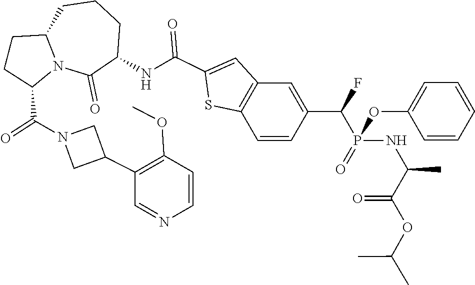 [SMILES: COc1ccncc1C1CN(C(=O)[C@@H]2CC[C@@H]3CCC[C@H](NC(=O)c4cc5cc([C@@H](F)[P@@](=O)(N[C@@H](C)C(=O)OC(C)C)Oc6ccccc6)ccc5s4)C(=O)N32)C1] More polar linker building block |
| | (4-methoxypyridin- | used for synthesis and biological |
| | 3-yl)azetidine-1- | testing |
| | carbonyl)-5- | |
| | oxooctahydro-1H- | |
| | pyrrolo[1,2- | |
| | a]azepin-6- | |
| | yl)carbamoyl)benzo | |
| | [b]thiophen-5- | |
| | yl)methyl)(phenoxy) | |
| | phosphoryl)-L- | |
| | alaninate | |
| 957 | propyl ((fluoro(2- (((3S,6S,9aS)-3-(3- (5-fluoro-2- methoxyphenyl) azetidine- 1-carbonyl)- 5-oxooctahydro- 1H-pyrrolo[1,2- a]azepin-6- yl)carbamoyl)benzo [b]thiophen-5- yl)methyl)(phenoxy) phosphoryl)-L- alaninate | 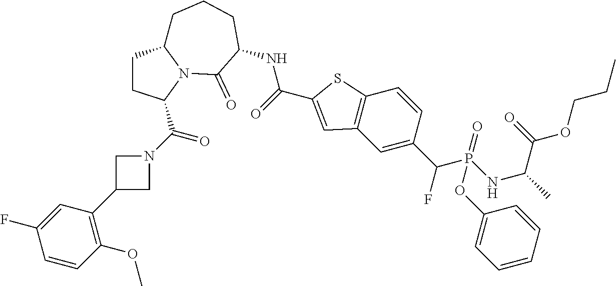 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cc(F)ccc6OC)C5)N4C3=O)cc2c1] |
| 958 | propyl (((2- (((3S,6S,10aS)-3- (((3S,4S)-1- carbamoyl-4- fluoropyrrolidin-3- yl)carbamoyl)-5- oxodeca- hydropyrrolo [1,2-a]azocin-6- yl)carbamoyl) benzo [b]thiophen-5- yl)fluoromethyl) (phenoxy) phosphoryl)- L-alaninate | 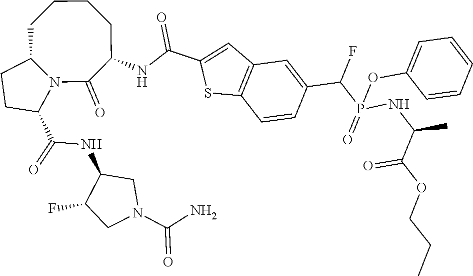 [SMILES: CCCOC(=O)[C@H](C)NP(=O)(Oc1ccccc1)C(F)c1ccc2sc(C(=O)N[C@H]3CCCC[C@H]4CC[C@@H](C(=O)N[C@H]5CN(C(N)=O)C[C@@H]5F)N4C3=O)cc2c1] |
| 959 | neopentyl ((R)- ((R)-fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate or neopentyl ((S)-((R)- fluoro(2- (((3S,6S,9aS)-3-(3- (4-methoxypyridin- 3-yl)azetidine-1- | 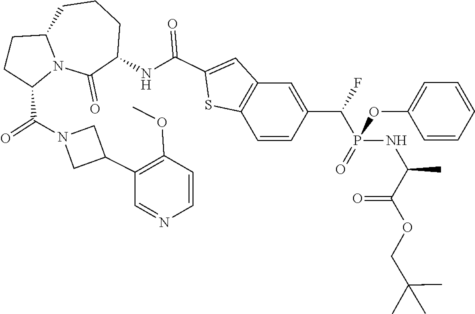 [SMILES: COc1ccncc1C1CN(C(=O)[C@@H]2CC[C@@H]3CCC[C@H](NC(=O)c4cc5cc([C@H](F)[P@@](=O)(N[C@@H](C)C(=O)OCC(C)(C)C)Oc6ccccc6)ccc5s4)C(=O)N32)C1] |
| | carbonyl)-5- | Less polar linker building block |
| | oxooctahydro-1H- | used for synthesis and biological |
| | pyrrolo[1,2- | testing |
| | a]azepin-6- | |
| | yl)carbamoyl)benzo | |
| | [b]thiophen-5- | |
| | yl)methyl)(phenoxy) | |
| | phosphoryl)-L- | |
| | alaninate | |
| 960 | neopentyl ((R)- ((R)-fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl) (phenoxy) phosphoryl)-L- alaninate | 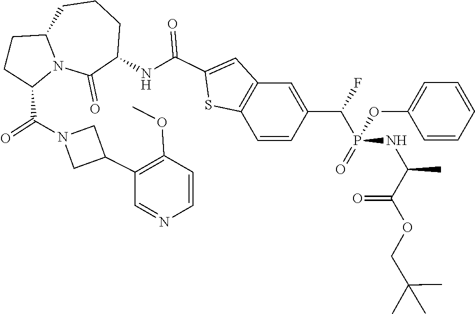 [SMILES: COc1ccncc1C1CN(C(=O)[C@@H]2CC[C@@H]3CCC[C@H](NC(=O)c4cc5cc([C@H](F)[P@](=O)(N[C@@H](C)C(=O)OCC(C)(C)C)Oc6ccccc6)ccc5s4)C(=O)N32)C1] |
| | or | or |
| | neopentyl ((S)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 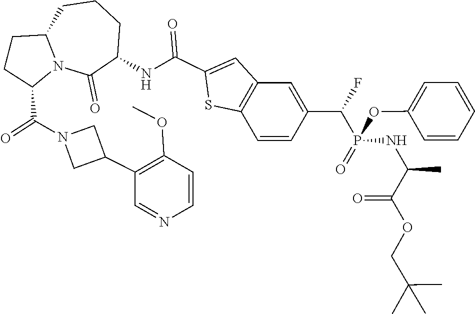 [SMILES: COc1ccncc1C1CN(C(=O)[C@@H]2CC[C@@H]3CCC[C@H](NC(=O)c4cc5cc([C@H](F)[P@@](=O)(N[C@@H](C)C(=O)OCC(C)(C)C)Oc6ccccc6)ccc5s4)C(=O)N32)C1] |
| | | More polar linker building block |
| | | used for synthesis and biological |
| | | testing |
| 961 | S,S′-((((difluoro(2- (((3S,6S,9aS)-5- oxo-3-(3- (pyrimidin-2- yl)azetidine-1- carbonyl)octahydro- 1H-pyrrolo[1,2- a]azepin-6- yl)carbamoyl)benzo [b]thiophen-5- yl)methyl)phosphoryl) bis(oxy))bis(ethane- 2,1-diyl)) bis(3- methylbutanethioate) | 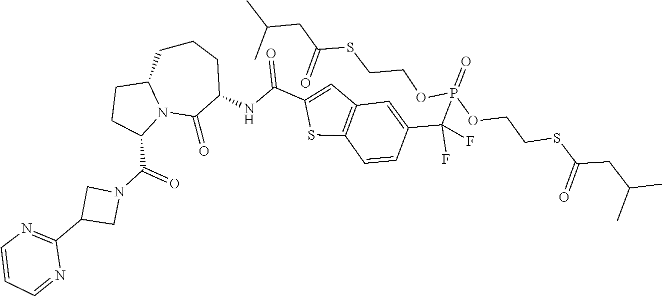 [SMILES: CC(C)CC(=O)SCCOP(=O)(OCCSC(=O)CC(C)C)C(F)(F)c1ccc2sc(C(=O)N[C@H]3CCC[C@H]4CC[C@@H](C(=O)N5CC(c6ncccn6)C5)N4C3=O)cc2c1] |
| 962 | S,S′-((((difluoro(2- (((3S,6S,9aS)-5- oxo-3-(3- (pyrimidin-2- yl)azetidine-1- carbonyl)octahydro- 1H-pyrrolo[1,2- a]azepin-6- yl)carbamoyl)benzo [b]thiophen-5- yl)methyl) phosphoryl) bis(oxy))bis(ethane- 2,1-diyl))bis(2,2- dimethyl- propanethioate) | 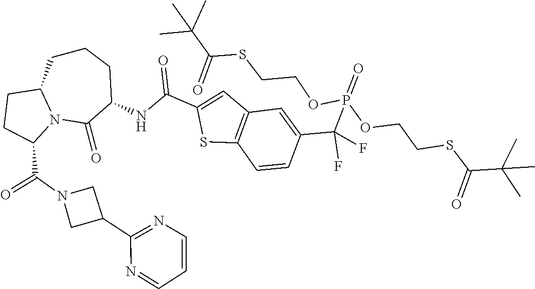 [SMILES: CC(C)(C)C(=O)SCCOP(=O)(OCCSC(=O)C(C)(C)C)C(F)(F)c1ccc2sc(C(=O)N[C@H]3CCC[C@H]4CC[C@@H](C(=O)N5CC(c6ncccn6)C5)N4C3=O)cc2c1] |
| 963 | S,S′-((((difluoro(2- (((3S,6S,9aS)-3-(3- methoxy-3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphoryl) | 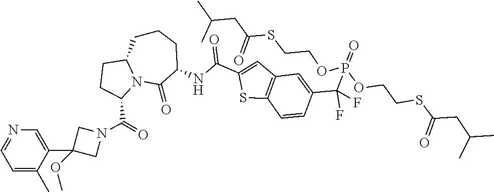 [SMILES: COC1(c2cnccc2C)CN(C(=O)[C@@H]2CC[C@@H]3CCC[C@H](NC(=O)c4cc5cc(C(F)(F)P(=O)(OCCSC(=O)CC(C)C)OCCSC(=O)CC(C)C)ccc5s4)C(=O)N32)C1] |
| | bis(oxy))bis(ethane- | |
| | 2,1-diyl)) bis(3- | |
| | methylbutanethioate) | |
| 964 | S,S′-((((difluoro(2- (((3S,6S,9aS)-3-(3- (4-methyl-4H- 1,2,4-triazol-3- yl)azetidine-1- carbonyl)-5- oxooctahydro-1H- pyrrolo[1,2- a]azepin-6- yl)carbamoyl)benzo [b]thiophen-5- yl)methyl) phosphoryl) bis(oxy))bis(ethane- 2,1-diyl)) dibutanethioate | 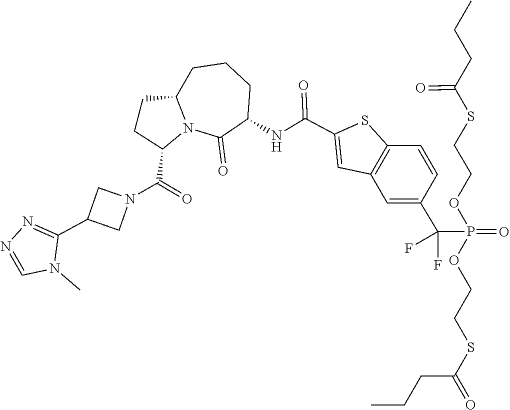 [SMILES: CCCC(=O)SCCOP(=O)(OCCSC(=O)CCC)C(F)(F)c1ccc2sc(C(=O)N[C@H]3CCC[C@H]4CC[C@@H](C(=O)N5CC(c6nncn6C)C5)N4C3=O)cc2c1] |
| 965 | diisopropyl (((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)-1H- indol-5- yl)methyl)phosphoryl) bis(oxy))bis (methylene)) bis(carbonate) | 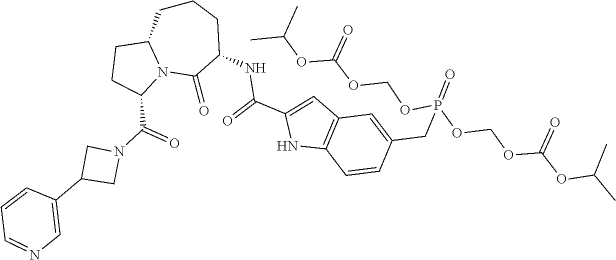 [SMILES: CC(C)OC(=O)OCOP(=O)(Cc1ccc2[nH]c(C(=O)N[C@H]3CCC[C@H]4CC[C@@H](C(=O)N5CC(c6cccnc6)C5)N4C3=O)cc2c1)OCOC(=O)OC(C)C] |
| 966 | S,S′-((((difluoro(2- (((3S,6S,9aS)-3-(3- (4-methyl-4H- 1,2,4-triazol-3- yl)azetidine-1- carbonyl)-5- oxooctahydro-1H- pyrrolo[1,2- a]azepin-6- yl)carbamoyl)benzo [b]thiophen-5- yl)methyl)phosphoryl) bis(oxy)) bis(ethane- 2,1-diyl)) bis(3- methylbutanethioate) | 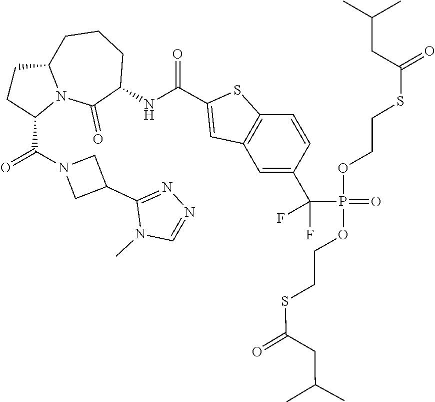 [SMILES: CC(C)CC(=O)SCCOP(=O)(OCCSC(=O)CC(C)C)C(F)(F)c1ccc2sc(C(=O)N[C@H]3CCC[C@H]4CC[C@@H](C(=O)N5CC(c6nncn6C)C5)N4C3=O)cc2c1] |
| 967 | S,S′-((((difluoro(2- (((3S,6S,9aS)-3-(3- (4-methyl-4H- 1,2,4-triazol-3- yl)azetidine-1- carbonyl)-5- oxooctahydro-1H- pyrrolo[1,2- a]azepin-6- yl)carbamoyl)benzo [b]thiophen-5- yl)methyl) phosphoryl) bis(oxy))bis(ethane- 2,1-diyl)) bis(2,2- dimethyl- propanethioate) | 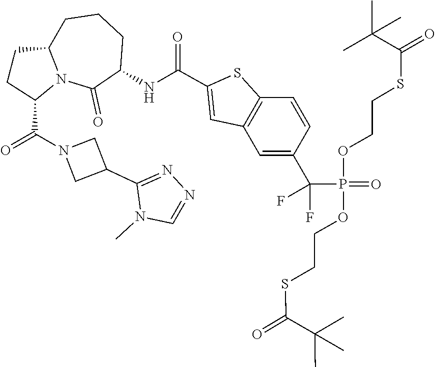 [SMILES: Cn1cnnc1C1CN(C(=O)[C@@H]2CC[C@@H]3CCC[C@H](NC(=O)c4cc5cc(C(F)(F)P(=O)(OCCSC(=O)C(C)(C)C)OCCSC(=O)C(C)(C)C)ccc5s4)C(=O)N32)C1] |
| 968 | S,S′-((((difluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl) phosphoryl) bis(oxy))bis(ethane- 2,1-diyl))bis(2,2- dimethyl- propanethioate) | 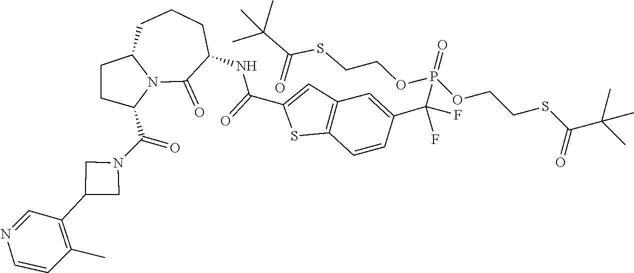 [SMILES: Cc1ccncc1C1CN(C(=O)[C@@H]2CC[C@@H]3CCC[C@H](NC(=O)c4cc5cc(C(F)(F)P(=O)(OCCSC(=O)C(C)(C)C)OCCSC(=O)C(C)(C)C)ccc5s4)C(=O)N32)C1] |
| 969 | butyl ((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- prolinate | 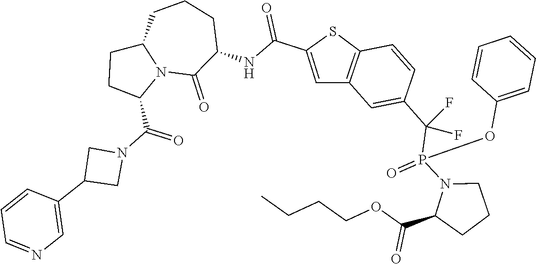 [SMILES: CCCCOC(=O)[C@@H]1CCCN1P(=O)(Oc1ccccc1)C(F)(F)c1ccc2sc(C(=O)N[C@H]3CCC[C@H]4CC[C@@H](C(=O)N5CC(c6cccnc6)C5)N4C3=O)cc2c1] |
| 970 | S,S′-((((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)- phosphoryl) bis(oxy))bis(ethane- 2,1-diyl)) dibenzothioate | 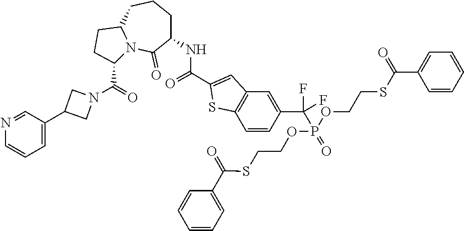 |
| 971 | butyl ((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl) (naphthalen-1- yloxy)phosphoryl)- L-alaninate | 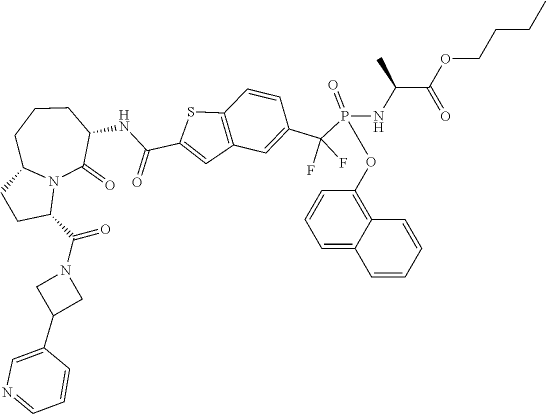 [SMILES: CCCCOC(=O)[C@H](C)NP(=O)(Oc1cccc2ccccc12)C(F)(F)c1ccc2sc(C(=O)N[C@H]3CCC[C@H]4CC[C@@H](C(=O)N5CC(c6cccnc6)C5)N4C3=O)cc2c1] |
| 972 | isopropyl ((fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl) (phenoxy) phosphoryl)- | 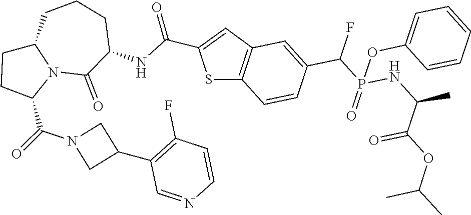 [SMILES: CC(C)OC(=O)[C@H](C)NP(=O)(Oc1ccccc1)C(F)c1ccc2sc(C(=O)N[C@H]3CCC[C@H]4CC[C@@H](C(=O)N5CC(c6cnccc6F)C5)N4C3=O)cc2c1] |
| | L-alaninate | |
| 973 | propyl (((2- (((3S,6S,9aS)-3-(3- (4- (dimethylamino) pyridin- 3-yl)azetidine- 1-carbonyl)-5- oxooctahydro-1H- pyrrolo[1,2- a]azepin-6- yl)carbamoyl)benzo [b]thiophen-5- yl)methyl)(phenoxy )phosphoryl)-L- alaninate | 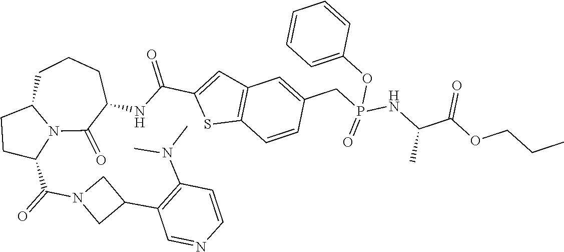 [SMILES: CCCOC(=O)[C@H](C)NP(=O)(Cc1ccc2sc(C(=O)N[C@H]3CCC[C@H]4CC[C@@H](C(=O)N5CC(c6cnccc6N(C)C)C5)N4C3=O)cc2c1)Oc1ccccc1] |
| 974 | propyl ((fluoro(2- (((3S,6S,9aS)-3-(3- (4-hydroxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 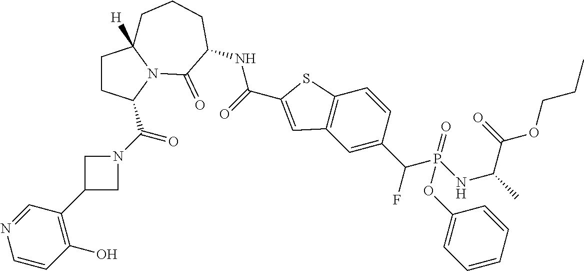 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cnccc6O)C5)N4C3=O)cc2c1] |
| 975 | propyl (((2- (((3S,6S,9aS)-3-(3- (4-hydroxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 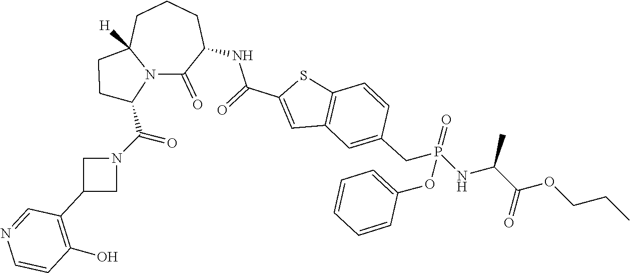 [SMILES: CCCOC(=O)[C@H](C)NP(=O)(Cc1ccc2sc(C(=O)N[C@H]3CCC[C@H]4CC[C@@H](C(=O)N5CC(c6cnccc6O)C5)N4C3=O)cc2c1)Oc1ccccc1] |
| 976 | propyl ((fluoro(2- (((3S,6S,10aS)-3- (6-(5-fluoro-1- methyl-2-oxo-1,2- dihydropyridin-4- yl)-5,7-dioxo-4,6- diazaspiro [2.4]heptane- 4-carbonyl)-5- oxodecahydropyrrolo [1,2-a]azocin-6- yl)carbamoyl)benzo [b]thiophen-5- yl)methyl)(phenoxy) phosphoryl)-L- alaninate | 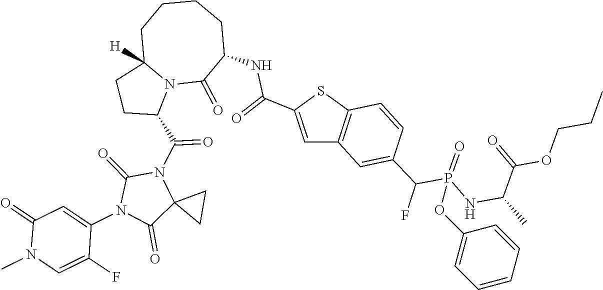 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6cc(=O)n(C)cc6F)C(=O)C56CC6)N4C3=O)cc2c1] |
| 977 | propyl ((fluoro(2- (((3S,6S,10aS)-3- (6-(1-methyl-2- oxo-1,2- dihydropyridin-4- yl)-5,7-dioxo-4,6- diazaspiro[2.4] heptane-4- carbonyl)-5- oxodecahydropyrrolo [1,2-a]azocin-6- yl)carbamoyl)benzo [b]thiophen-5- yl)methyl)(phenoxy) phosphoryl)-L- alaninate | 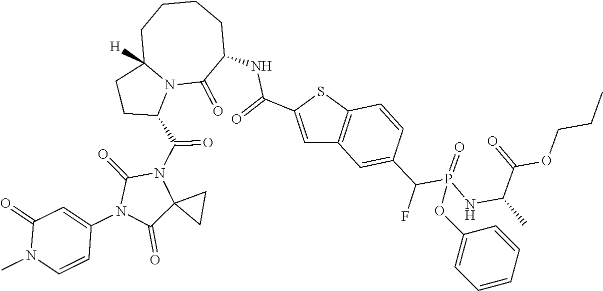 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6ccn(C)c(=O)c6)C(=O)C56CC6)N4C3=O)cc2c1] |
| 978 | propyl (((2- (((3S,6S,9aS)-3-(3- (4-fluoro-2- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 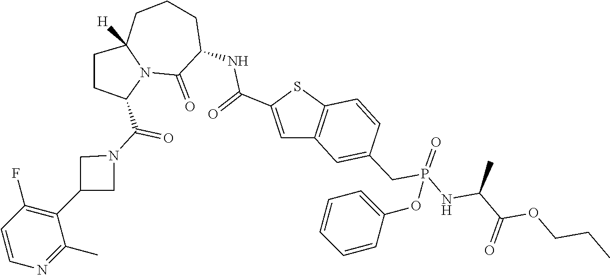 [SMILES: CCCOC(=O)[C@H](C)NP(=O)(Cc1ccc2sc(C(=O)N[C@H]3CCC[C@H]4CC[C@@H](C(=O)N5CC(c6c(F)ccnc6C)C5)N4C3=O)cc2c1)Oc1ccccc1] |
| 979 | propyl (((2- (((3S,6S,9aS)-3-(3- (1-methyl-4-oxo- 1,4-dihydropyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 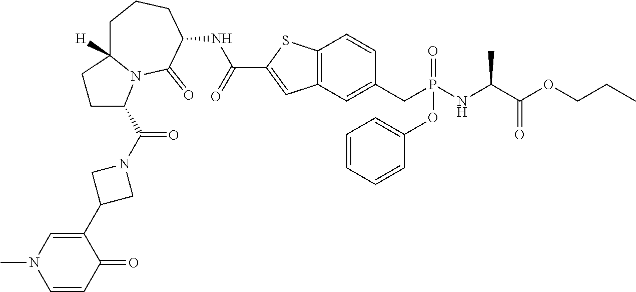 [SMILES: CCCOC(=O)[C@H](C)NP(=O)(Cc1ccc2sc(C(=O)N[C@H]3CCC[C@H]4CC[C@@H](C(=O)N5CC(c6cn(C)ccc6=O)C5)N4C3=O)cc2c1)Oc1ccccc1] |
| 980 | propyl (((2- (((3S,6S,9aS)-3-(3- (2-fluoro-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 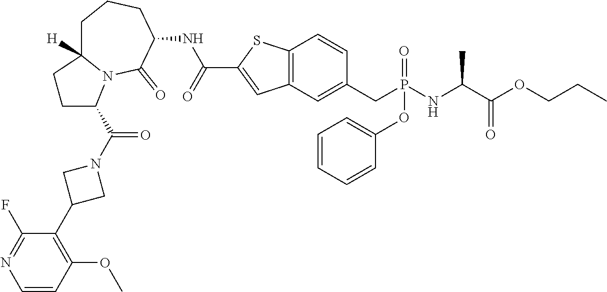 [SMILES: CCCOC(=O)[C@H](C)NP(=O)(Cc1ccc2sc(C(=O)N[C@H]3CCC[C@H]4CC[C@@H](C(=O)N5CC(c6c(OC)ccnc6F)C5)N4C3=O)cc2c1)Oc1ccccc1] |
| 981 | propyl ((fluoro(2- (((3S,6S,10aS)-3- (6-(1-isopropyl-2- oxo-1,2- dihydropyridin-4- yl)-5,7-dioxo-4,6- diazaspiro [2.4]heptane- 4-carbonyl)-5- oxodecahydropyrrolo [1,2-a]azocin-6- yl)carbamoyl)benzo [b]thiophen-5- yl)methyl)(phenoxy) phosphoryl)-L- alaninate | 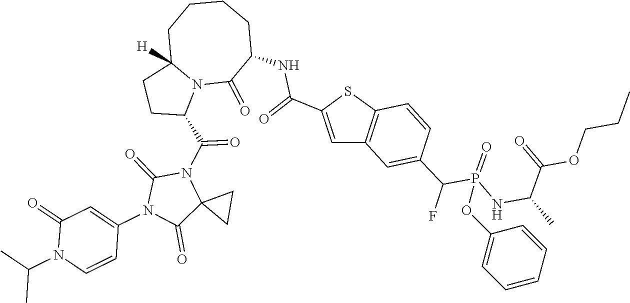 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6ccn(C(C)C)c(=O)c6)C(=O)C56CC6)N4C3=O)cc2c1] |
| 982 | propyl (((2- (((3S,6S,10aS)-3- (6-(2,2- difluorobenzo[d][1, 3]dioxol-4-yl)-5,7- dioxo-4,6- diazaspiro [2.4]heptane- 4-carbonyl)-5- oxodecahydropyrrolo [1,2-a]azocin-6- yl)carbamoyl)benzo [b]thiophen-5- yl)fluoromethyl) (phenoxy) phosphoryl)- L-alaninate | 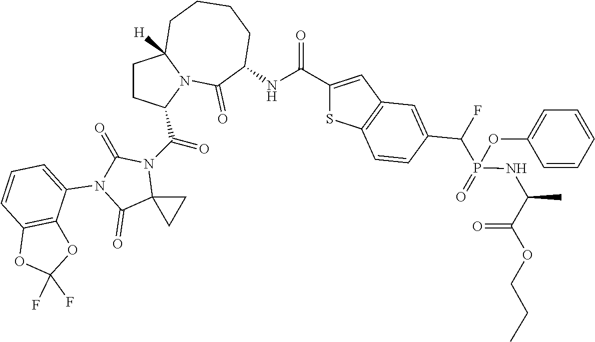 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6cccc7c6OC(F)(F)O7)C(=O)C56CC6)N4C3=O)cc2c1] |
| 983 | 3,3,3- trifluoropropyl ((fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy )phosphoryl)-L- alaninate | 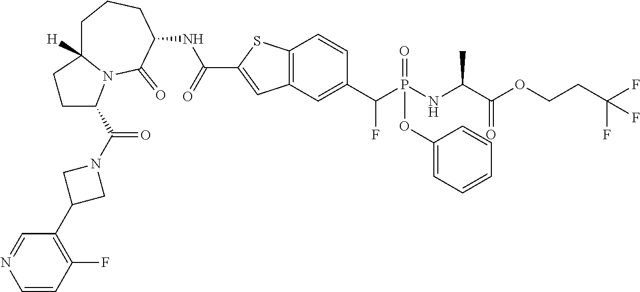 [SMILES: C[C@H](NP(=O)(Oc1ccccc1)C(F)c1ccc2sc(C(=O)N[C@H]3CCC[C@H]4CC[C@@H](C(=O)N5CC(c6cnccc6F)C5)N4C3=O)cc2c1)C(=O)OCCC(F)(F)F] |
| 984 | S,S′-((((fluoro(2- (((3S,6S,9aS)-3-(3- (4-fluoropyridin-3- yl)azetidine- 1-carbonyl)-5- oxooctahydro-1H- pyrrolo[1,2- a]azepin-6- yl)carbamoyl)benzo [b]thiophen-5- yl)methyl) phosphoryl) bis(oxy))bis(ethane- 2,1-diyl))bis(2,2- dimethyl- propanethioate) | 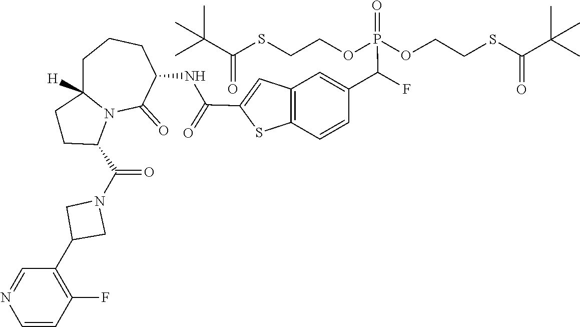 [SMILES: CC(C)(C)C(=O)SCCOP(=O)(OCCSC(=O)C(C)(C)C)C(F)c1ccc2sc(C(=O)N[C@H]3CCC[C@H]4CC[C@@H](C(=O)N5CC(c6cnccc6F)C5)N4C3=O)cc2c1] |
| 985 | propyl ((fluoro(2- (((3S,6S,9aS)-3-(3- (1-methyl-4-oxo- 1,4-dihydropyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 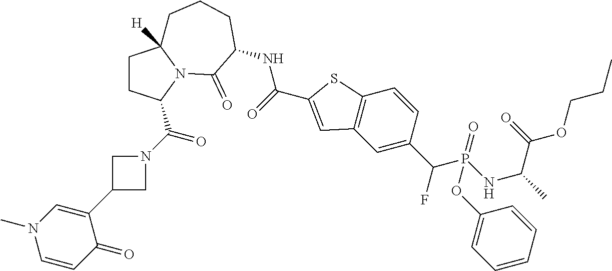 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cn(C)ccc6=O)C5)N4C3=O)cc2c1] |
| 986 | propyl (((2- (((3S,6S,9aS)-3-(3- (2,5- difluorophenyl) azetidine-1-carbonyl)- 5-oxooctahydro- 1H-pyrrolo[1,2- a]azepin-6- yl)carbamoyl)benzo [b]thiophen-5- yl)fluoromethyl) (phenoxy)phosphoryl)- L-alaninate | 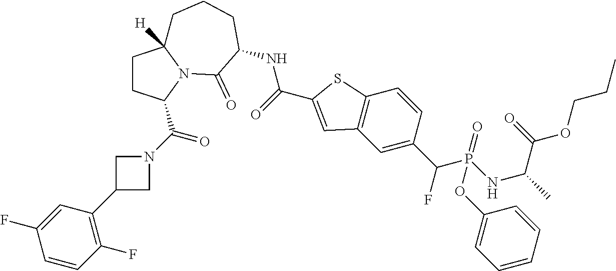 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cc(F)ccc6F)C5)N4C3=O)cc2c1] |
| 987 | propyl ((fluoro(2- (((3S,6S,10aS)-3- (3-((2- methoxyethyl)amino)- 3-(pyridin-2- yl)azetidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 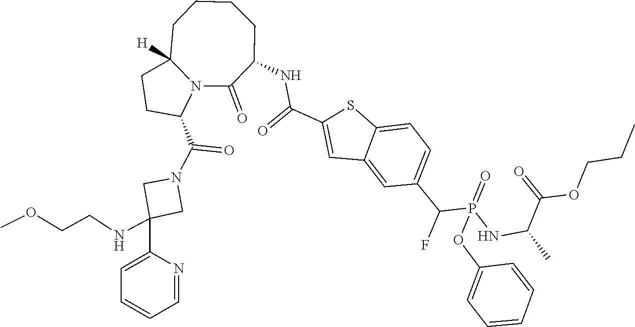 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CC(NCCOC)(c6ccccn6)C5)N4C3=O)cc2c1] |
| 988 | propyl (((2- (((3S,6S,9R,10aR)- 3-(3-(4- fluoropyridin-3- yl)azetidine-1- carbonyl)-9- methyl-5- oxodecahydropyrrolo [1,2-a]azocin-6- yl)carbamoyl)benzo [b]thiophen-5- yl)methyl)(phenoxy) phosphoryl)-L- alaninate | 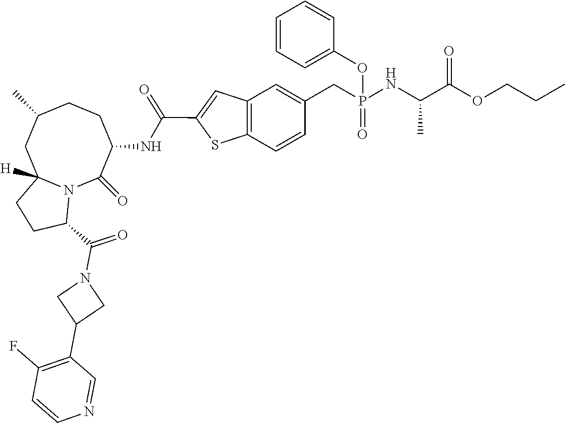 [SMILES: CCCOC(=O)[C@H](C)NP(=O)(Cc1ccc2sc(C(=O)N[C@H]3CC[C@@H](C)C[C@H]4CC[C@@H](C(=O)N5CC(c6cnccc6F)C5)N4C3=O)cc2c1)Oc1ccccc1] |
| 989 | propyl (((2- (((3S,6S,10aS)-3- (5,5-dimethyl-2,4- dioxo-3-(pyridin-3- yl)imidazolidine-1- carbonyl)-5- oxodecahydropyrrolo [1,2-a]azocin-6- yl)carbamoyl)benzo [b]thiophen-5- yl)fluoromethyl) (phenoxy)phosphoryl)- L-alaninate | 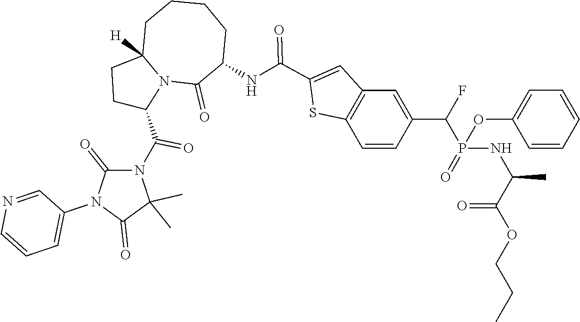 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6cccnc6)C(=O)C5(C)C)N4C3=O)cc2c1] |
| 990 | propyl (((2- (((3S,6S, 9aS)-3-(3-(2- (difluoromethyl)-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 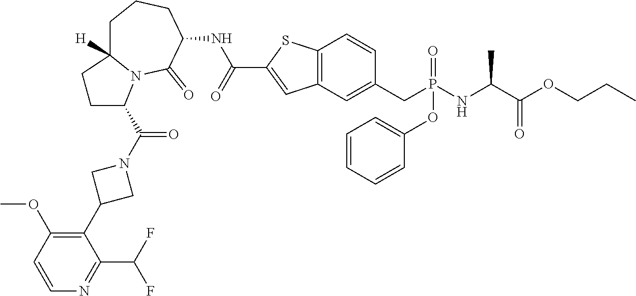 [SMILES: CCCOC(=O)[C@H](C)NP(=O)(Cc1ccc2sc(C(=O)N[C@H]3CCC[C@H]4CC[C@@H](C(=O)N5CC(c6c(OC)ccnc6C(F)F)C5)N4C3=O)cc2c1)Oc1ccccc1] |
| 991 | propyl (((2- (((3S,6S,9aS)-3-(3- (6- (difluoromethyl)-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 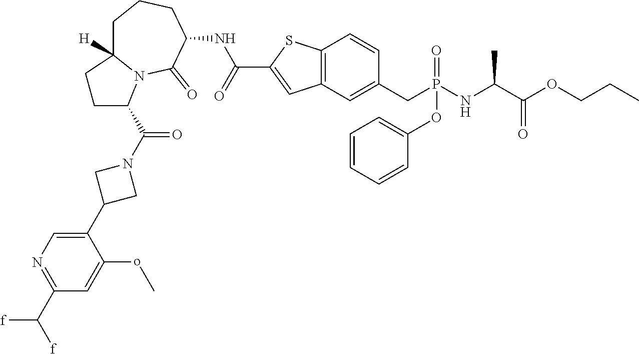 |
| 992 | propyl (((2- (((3S,6S,10aS)-3- (6,8-dioxo-7- (pyridin-3-yl)-5,7- diazaspiro[3.4] octane- 5-carbonyl)-5- oxodecahydropyrrolo [1,2-a]azocin-6- yl)carbamoyl)benzo [b]thiophen-5- yl)fluoromethyl) (phenoxy) phosphoryl)- L-alaninate | 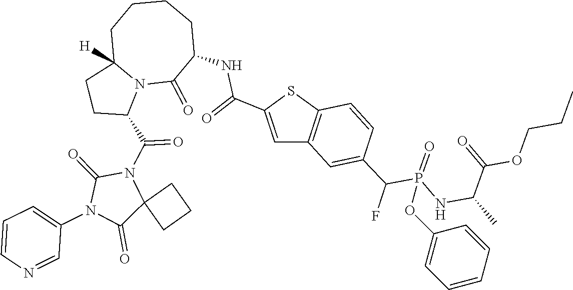 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5C(=O)N(c6cccnc6)C(=O)C56CCC6)N4C3=O)cc2c1] |
| 993 | propyl (((2- (((3S,6S,9S,10aR)- 3-(3-(4- fluoropyridin-3- yl)azetidine-1- carbonyl)-9- methyl-5- oxodecahydropyrrolo [1,2-a]azocin-6- yl)carbamoyl)benzo [b]thiophen-5- yl)methyl)(phenoxy) phosphoryl)-L- alaninate | 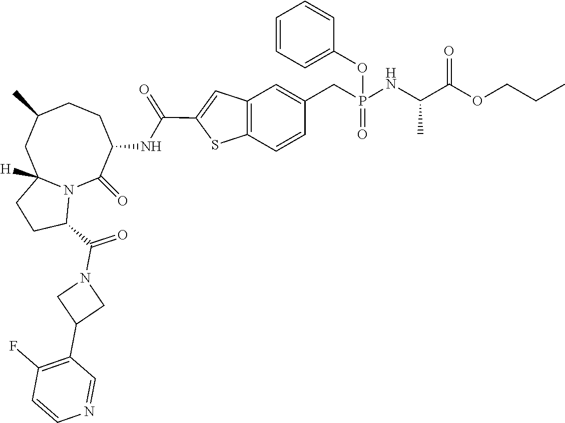 [SMILES: CCCOC(=O)[C@H](C)NP(=O)(Cc1ccc2sc(C(=O)N[C@H]3CC[C@H](C)C[C@H]4CC[C@@H](C(=O)N5CC(c6cnccc6F)C5)N4C3=O)cc2c1)Oc1ccccc1] |
| 994 | propyl (((R)- fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 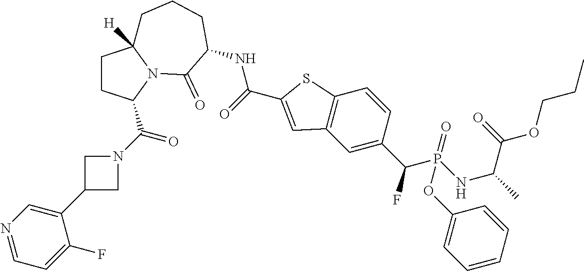 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cnccc6F)C5)N4C3=O)cc2c1] |
| 995 | propyl ((fluoro(2- (((3S,6S,10aS)-3- (3-(2- methoxyethoxy)-3- (pyridin-3- yl)azetidine-1- carbonyl)-5- oxodecahydropyrrolo [1,2-a]azocin-6- yl)carbamoyl)benzo [b]thiophen-5- yl)methyl)(phenoxy) phosphoryl)-L- alaninate | 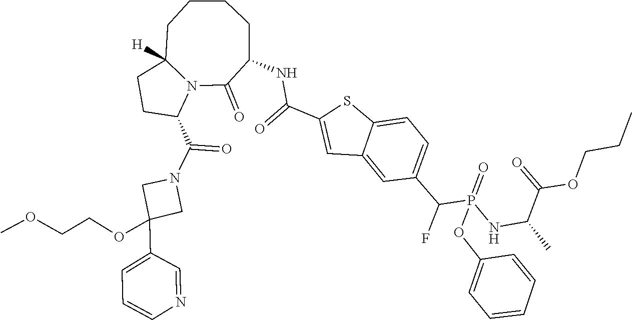 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CC(OCCOC)(c6cccnc6)C5)N4C3=O)cc2c1] |
| 996 | propyl (((2- (((3S,6S,9aS)-3-(3- (4- (difluoromethoxy) pyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) )phosphoryl)-L- alaninate | 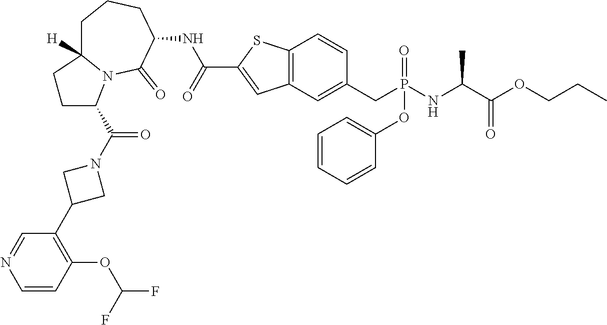 [SMILES: CCCOC(=O)[C@H](C)NP(=O)(Cc1ccc2sc(C(=O)N[C@H]3CCC[C@H]4CC[C@@H](C(=O)N5CC(c6cnccc6OC(F)F)C5)N4C3=O)cc2c1)Oc1ccccc1] |
| 997 | propyl ((R)-((S)- fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 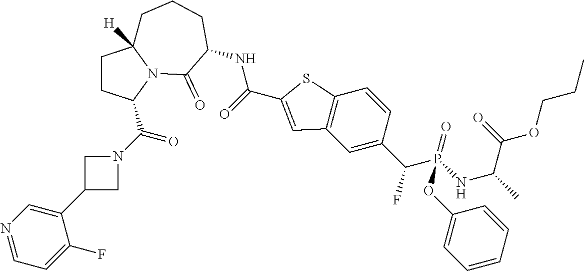 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cnccc6F)C5)N4C3=O)cc2c1] |
| | | or |
| | propyl ((S)-((S)- fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 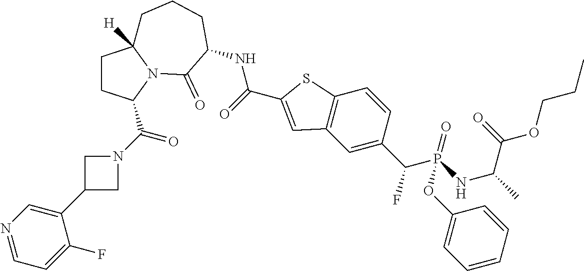 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cnccc6F)C5)N4C3=O)cc2c1] |
| | | CFTE-27A, SFC peak 1 was |
| | | used for synthesis and biological |
| | | testing |
| 998 | propyl ((R)-((S)- fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 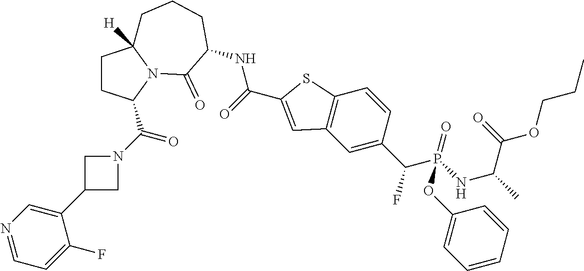 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cnccc6F)C5)N4C3=O)cc2c1] |
| | or | or |
| | propyl ((S)-((S)- fluoro(2- (((3S,6S,9aS)-3-(3- (4-fluoropyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 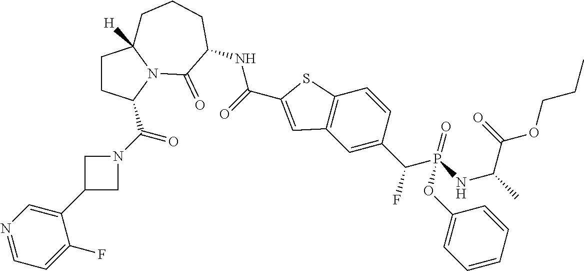 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cnccc6F)C5)N4C3=O)cc2c1] |
| | | CFTE-27B, SFC peak 2 was |
| | | used for synthesis and biological |
| | | testing |
| 999 | propyl ((R)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 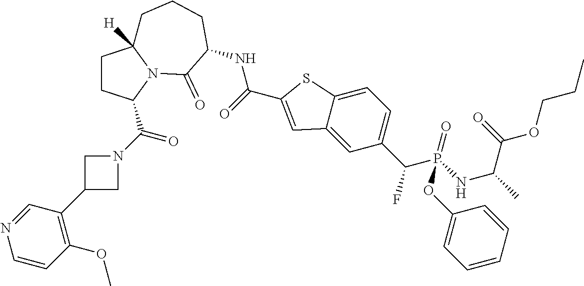 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cnccc6OC)C5)N4C3=O)cc2c1] |
| | or | or |
| | propyl ((S)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 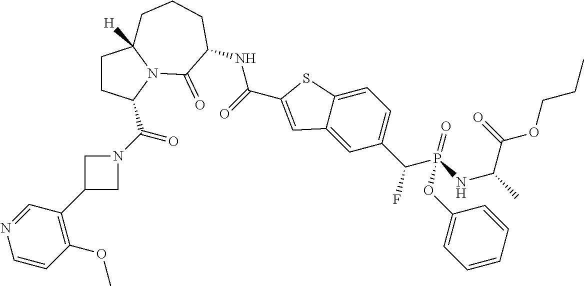 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cnccc6OC)C5)N4C3=O)cc2c1] |
| | | CFTE-27B, SFC peak 2 was |
| | | used for synthesis and biological |
| | | testing |
| 1000 | propyl ((S)-((S)- fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 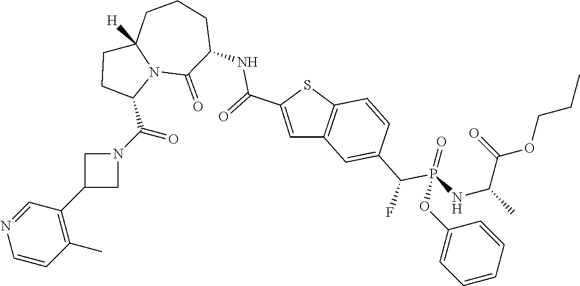 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cnccc6C)C5)N4C3=O)cc2c1] |
| | or | or |
| | propyl ((R)-((S)- fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 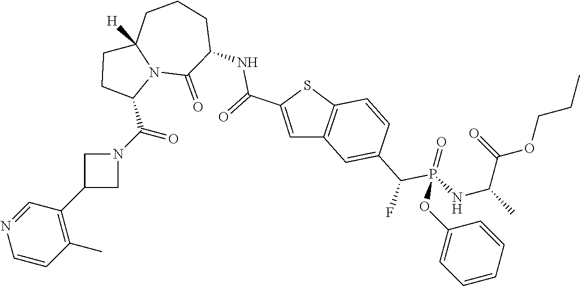 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cnccc6C)C5)N4C3=O)cc2c1] |
| | | CFTE-27B, SFC peak 2 was |
| | | used for synthesis and biological |
| | | testing |
| 1001 | propyl ((S)-((S)- fluoro(2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6-yl) carbamoyl)benzo [b]thiophen-5- yl)methyl)(phenoxy) phosphoryl)-L- alaninate | 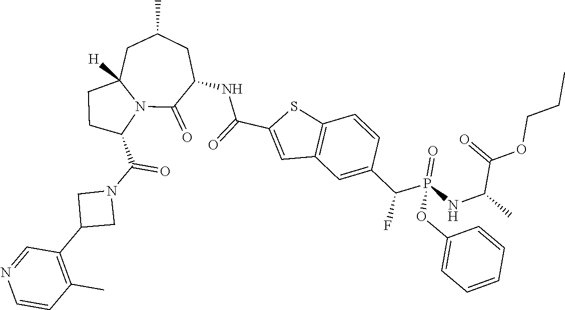 [SMILES: CCCOC(=O)[C@H](C)N[P@@](=O)(Oc1ccccc1)[C@H](F)c1ccc2sc(C(=O)N[C@H]3C[C@@H](C)C[C@H]4CC[C@@H](C(=O)N5CC(c6cnccc6C)C5)N4C3=O)cc2c1] |
| | or | or |
| | propyl ((R)-((S)- fluoro(2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 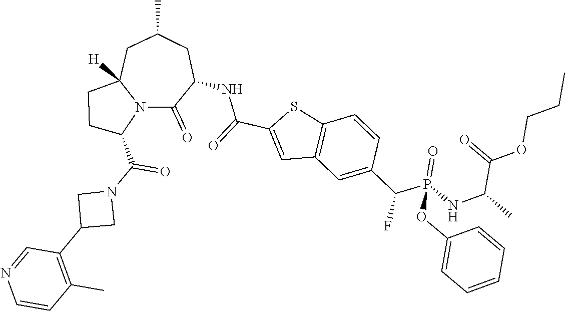 [SMILES: CCCOC(=O)[C@H](C)N[P@](=O)(Oc1ccccc1)[C@H](F)c1ccc2sc(C(=O)N[C@H]3C[C@@H](C)C[C@H]4CC[C@@H](C(=O)N5CC(c6cnccc6C)C5)N4C3=O)cc2c1] |
| | | CFTE-27B, SFC peak 2 was used for synthesis and biological testing |
| 1002 | S,S′-(((((2- (((3S,6S,9aS)-3- (((3H-imidazo[4,5- b]pyridin-6- yl)methyl) carbamoyl)-5- oxooctahydro-1H- pyrrolo[1,2- a]azepin-6- yl)carbamoyl)benzo [b]thiophen-5- yl)difluoromethyl) phosphoryl)bis(oxy)) bis(ethane-2,1- diyl)) bis(2,2- dimethyl- propanethioate) | 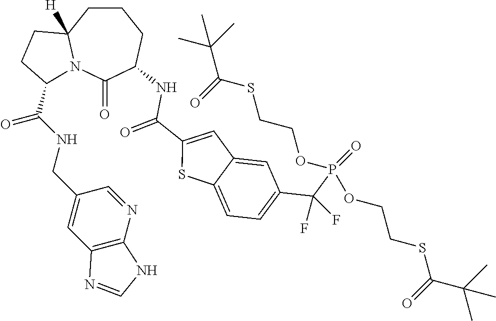 [SMILES: CC(C)(C)C(=O)SCCOP(=O)(OCCSC(=O)C(C)(C)C)C(F)(F)c1ccc2sc(C(=O)N[C@H]3CCC[C@H]4CC[C@@H](C(=O)NCc5cnc6[nH]cnc6c5)N4C3=O)cc2c1] |
| 1003 | butyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- prolinate | 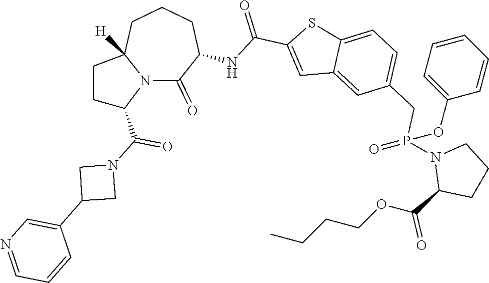 [SMILES: CCCCOC(=O)[C@@H]1CCCN1P(=O)(Cc1ccc2sc(C(=O)N[C@H]3CCC[C@H]4CC[C@@H](C(=O)N5CC(c6cccnc6)C5)N4C3=O)cc2c1)Oc1ccccc1] |
| 1004 | butyl ((naphthalen- 1-yloxy)((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl) phosphoryl)- L-alaninate | 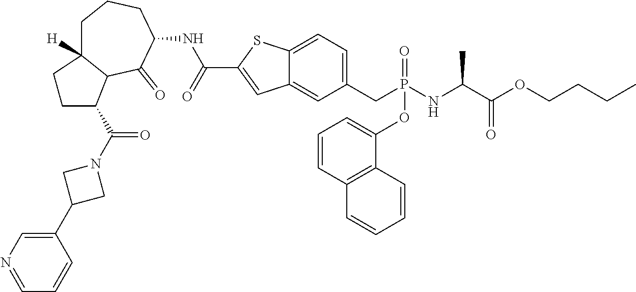 [SMILES: CCCCOC(=O)[C@H](C)NP(=O)(Cc1ccc2sc(C(=O)N[C@H]3CCC[C@H]4CC[C@@H](C(=O)N5CC(c6cccnc6)C5)C4C3=O)cc2c1)Oc1cccc2ccccc12] |
| 1005 | S,S′-(((((2- (((3S,6S,10aS)-3- (((3H-imidazo[4,5- b]pyridin-6- yl)methyl)carbamo yl)-5- oxodecahydropyrrolo [1,2-a]azocin-6- yl)carbamoyl)benzo [b]thiophen-5- yl)difluoromethyl) phosphoryl)bis(oxy)) bis(ethane-2,1- diyl)) bis(2,2- dimethyl- propanethioate) | 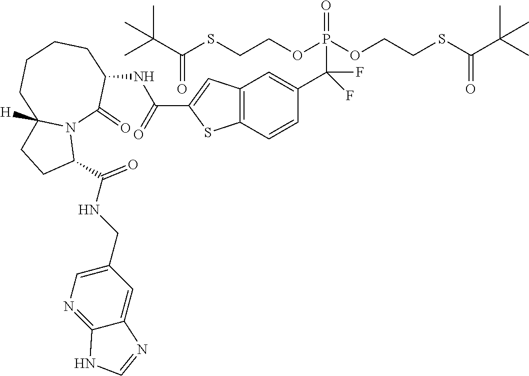 [SMILES: CC(C)(C)C(=O)SCCOP(=O)(OCCSC(=O)C(C)(C)C)C(F)(F)c1ccc2sc(C(=O)N[C@H]3CCCC[C@H]4CC[C@@H](C(=O)NCc5cnc6[nH]cnc6c5)N4C3=O)cc2c1] |
| 1006 | S,S′-((((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) bis(oxy))bis(ethane- 2,1-diyl)) bis(2- phenylethanethioate) | 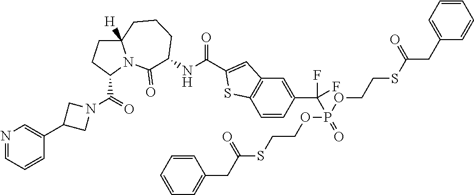 [SMILES: O=C(Cc1ccccc1)SCCOP(=O)(OCCSC(=O)Cc1ccccc1)C(F)(F)c1ccc2sc(C(=O)N[C@H]3CCC[C@H]4CC[C@@H](C(=O)N5CC(c6cccnc6)C5)N4C3=O)cc2c1] |
| 1007 | S-(2-((((2- (((3S,6S,10aS)-3- ((3R,4S)-3-cyano-4- phenylpyrrolidine- 1-carbonyl)-5- oxodecahydropyrrolo [1,2-a]azocin-6- yl)carbamoyl)benzo [b]thiophen-5- yl)difluoromethyl)(3- cyanophenoxy) phosphoryl) oxy)ethyl)3- methylbutanethioate | 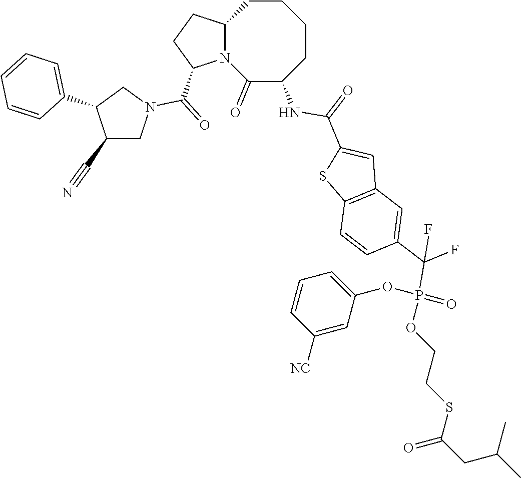 [SMILES: CC(C)CC(=O)SCCOP(=O)(Oc1cccc(C#N)c1)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] |
| | Or | Or |
| | S-(2-((((2- (((3S,6S,10aS)-3- ((3S,4R)-3-cyano-4- phenylpyrrolidine- 1-carbonyl)-5- oxodecahydropyrrolo [1,2-a]azocin-6- yl)carbamoyl)benzo [b]thiophen-5- yl)difluoromethyl)(3- cyanophenoxy)phos- phoryl)oxy)ethyl)3- methylbutanethioate | 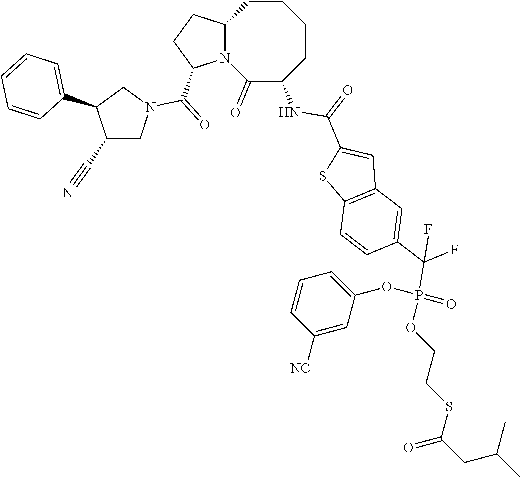 [SMILES: CC(C)CC(=O)SCCOP(=O)(Oc1cccc(C#N)c1)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] |
| | | Pyrrolidine building block SFC |
| | | peak 2 was used for synthesis |
| | | and biological testing |
| 1008 | S-(2-(((3- cyanophenoxy) (difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) oxy)ethyl) 3- methylbutanethioate | 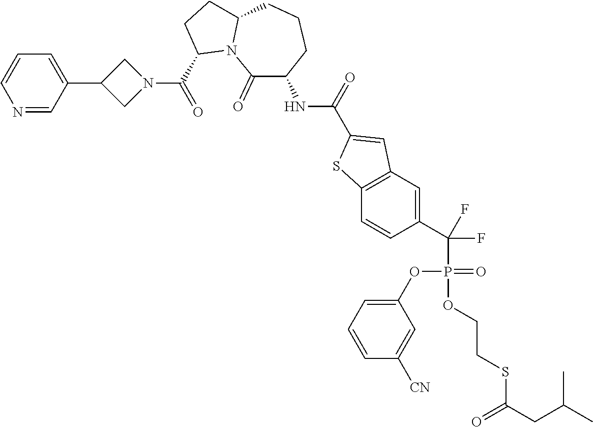 [SMILES: CC(C)CC(=O)SCCOP(=O)(Oc1cccc(C#N)c1)C(F)(F)c1ccc2sc(C(=O)N[C@H]3CCC[C@H]4CC[C@@H](C(=O)N5CC(c6cccnc6)C5)N4C3=O)cc2c1] |
| 1009 | S,S′-((((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl) phosphoryl) bis(oxy))bis(ethane- 2,1-diyl)) bis(2- methyl- propanethioate) | 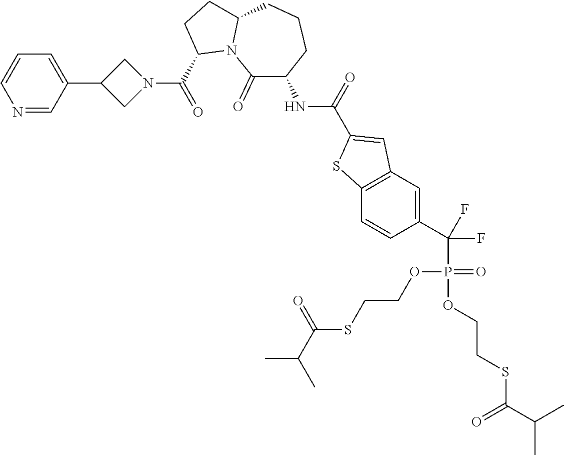 [SMILES: CC(C)C(=O)SCCOP(=O)(OCCSC(=O)C(C)C)C(F)(F)c1ccc2sc(C(=O)N[C@H]3CCC[C@H]4CC[C@@H](C(=O)N5CC(c6cccnc6)C5)N4C3=O)cc2c1] |
| 1010 | S,S′-(((((2- (((3S,6S,10aS)-3- ((3R,4S)-3-cyano- 4-(2-oxo-1,2- dihydropyridin-4- yl)pyrrolidine-1- carbonyl)-5- oxodecahydro- pyrrolo [1,2-a]azocin-6- yl)carbamoyl)benzo [b]thiophen-5- yl)difluoromethyl) phosphoryl) bis(oxy)) bis(ethane-2,1- diyl)) bis(2- methoxy- ethanethioate) | 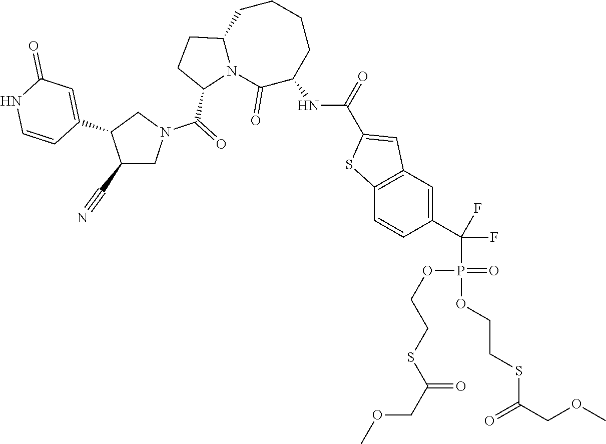 [SMILES: COCC(=O)SCCOP(=O)(OCCSC(=O)COC)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | Or | Or |
| | S,S′-(((((2- (((3S,6S,10aS)-3- ((3S,4R)-3-cyano- 4-(2-oxo-1,2- dihydropyridin-4- yl)pyrrolidine-1- carbonyl)-5- oxodeca- hydropyrrolo [1,2-a]azocin-6- yl)carbamoyl)benzo [b]thiophen-5- yl)difluoromethyl) phosphoryl)bis(oxy)) bis(ethane-2,1- diyl)) bis(2- methoxy- ethanethioate) | 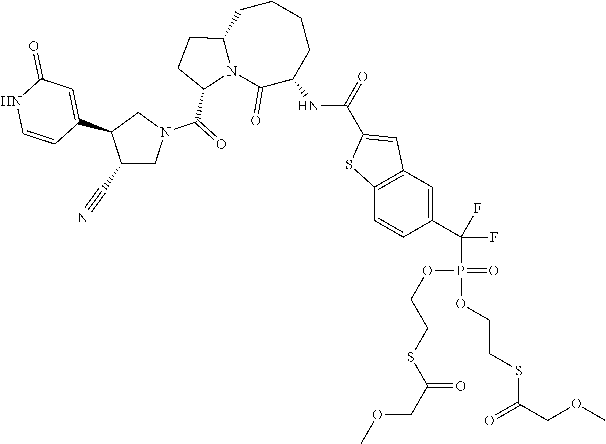 [SMILES: COCC(=O)SCCOP(=O)(OCCSC(=O)COC)C(F)(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | | Pyrrolidine building block SFC |
| | | peak 2 was used for synthesis |
| | | and biological testing |
| 1011 | S,S′-((((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) bis(oxy))bis(ethane- 2,1-diyl)) bis(2- methoxyethanethioate) | 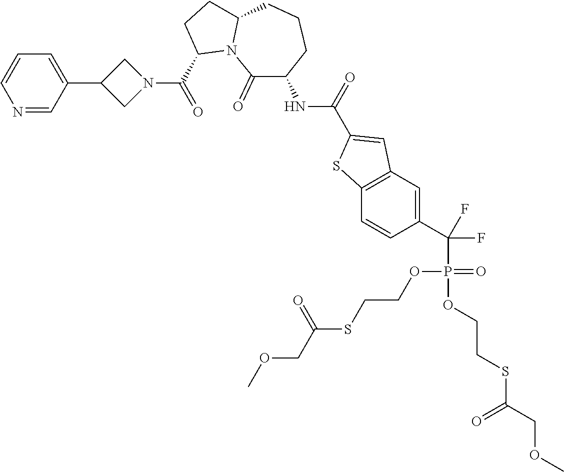 [SMILES: COCC(=O)SCCOP(=O)(OCCSC(=O)COC)C(F)(F)c1ccc2sc(C(=O)N[C@H]3CCC[C@H]4CC[C@@H](C(=O)N5CC(c6cccnc6)C5)N4C3=O)cc2c1] |
| 1012 | S-(2-((((2- (((3S,6S,10aS)-3- ((3R,4S)-3-cyano-4- phenylpyrrolidine- 1-carbonyl)-5- oxodecahydropyrrolo [1,2-a]azocin-6- yl)carbamoyl)benzo [b]thiophen-5- yl)difluoromethyl) (((isopropoxy- carbonyl) oxy)methoxy) phosphoryl)- oxy)ethyl) 2,2- dimethyl- propanethioate | 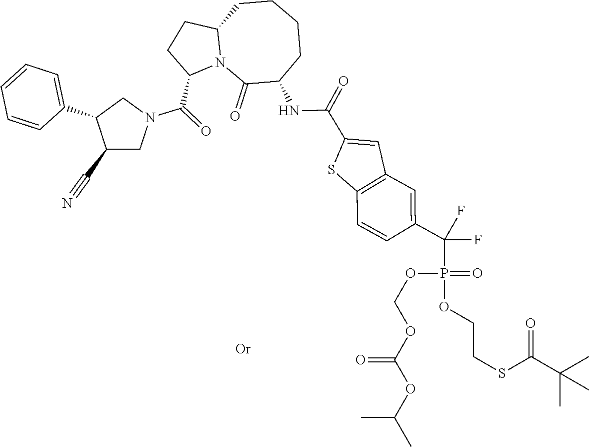 [SMILES: CC(C)OC(=O)OCOP(=O)(OCCSC(=O)C(C)(C)C)C(F)(F)c1ccc2sc(C(=O)N[C@H]3CCCC[C@H]4CC[C@@H](C(=O)N5C[C@@H](C#N)[C@H](c6ccccc6)C5)N4C3=O)cc2c1] |
| | Or | Or |
| | S-(2-((((2- (((3S,6S,10aS)-3- ((3S,4R)-3-cyano-4- phenylpyrrolidine- 1-carbonyl)-5- oxodecahydropyrrolo [1,2-a]azocin-6- yl)carbamoyl)benzo [b]thiophen-5- yl)difluoromethyl) (((isopropoxy- carbonyl) oxy)methoxy) phosphoryl)oxy)ethyl) 2,2- dimethyl- propanethioate | 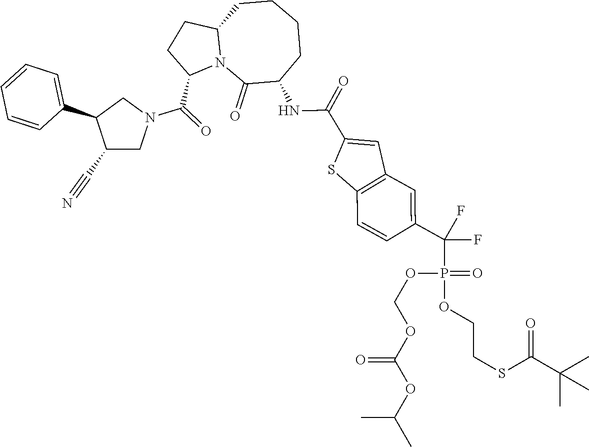 [SMILES: CC(C)OC(=O)OCOP(=O)(OCCSC(=O)C(C)(C)C)C(F)(F)c1ccc2sc(C(=O)N[C@H]3CCCC[C@H]4CC[C@@H](C(=O)N5C[C@H](c6ccccc6)[C@@H](C#N)C5)N4C3=O)cc2c1] |
| | | Pyrrolidine building block SFC |
| | | peak 2 was used for synthesis |
| | | and biological testing |
| 1013 | S,S′-(((((7- (((3S,6S,10aS)-3- ((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphoryl)bis(oxy)) bis(ethane-2,1- diyl)) bis(2-(2- methoxyethoxy) ethanethioate) Or | 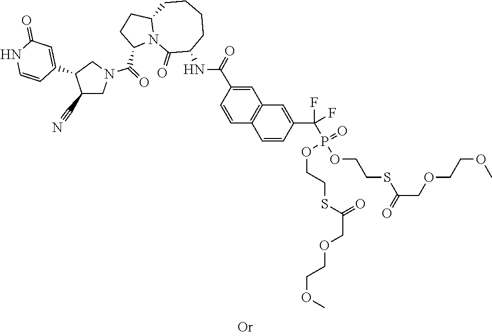 [SMILES: COCCOCC(=O)SCCOP(=O)(OCCSC(=O)COCCOC)C(F)(F)c1ccc2ccc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | S,S′-(((((7- (((3S,6S,10aS)-3- ((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphoryl) bis(oxy)) bis(ethane-2,1- diyl)) bis(2-(2- methoxyethoxy) ethanethioate) | 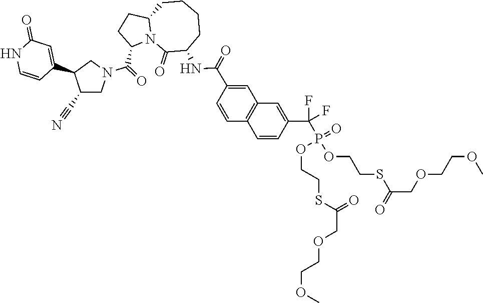 [SMILES: COCCOCC(=O)SCCOP(=O)(OCCSC(=O)COCCOC)C(F)(F)c1ccc2ccc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | | Pyrrolidine building block SFC |
| | | peak 2 was used for synthesis |
| | | and biological testing |
| 1014 | S,S′-((((difluoro(2- (((3S,6S,9aS)-3-(3- ((methylsulfonyl) methyl)azetidine-1- carbonyl)-5- oxooctahydro-1H- pyrrolo[1,2- a]azepin-6- yl)carbamoyl)benzo [b]thiophen-5- yl)methyl) phosphoryl) bis(oxy))bis(ethane- 2,1-diyl)) bis(2,2- dimethyl- propanethioate) | 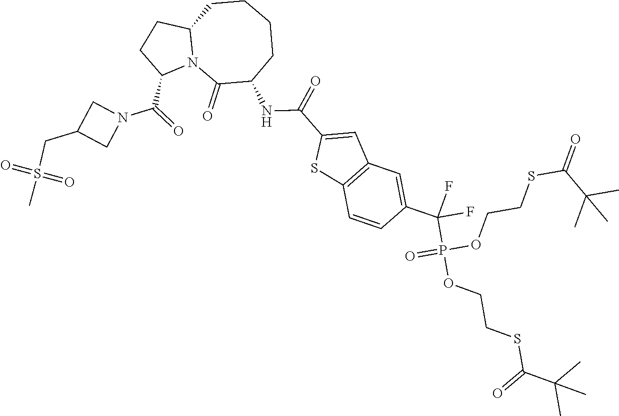 [SMILES: CC(C)(C)C(=O)SCCOP(=O)(OCCSC(=O)C(C)(C)C)C(F)(F)c1ccc2sc(C(=O)N[C@H]3CCCC[C@H]4CC[C@@H](C(=O)N5CC(CS(C)(=O)=O)C5)N4C3=O)cc2c1] |
| 1015 | S,S′-(((((2- (((3S,6S,9aS)-3-(3- ((N,S-dimethyl- sulfonimidoyl) methyl)-3- methoxyazetidine- 1-carbonyl)-5- oxooctahydro-1H- pyrrolo[1,2-a] azepin-6- yl)carbamoyl)benzo [b]thiophen-5- yl)difluoromethyl) phosphoryl) bis(oxy)) bis(ethane-2,1- diyl)) bis(2,2- dimethyl- propanethioate) | 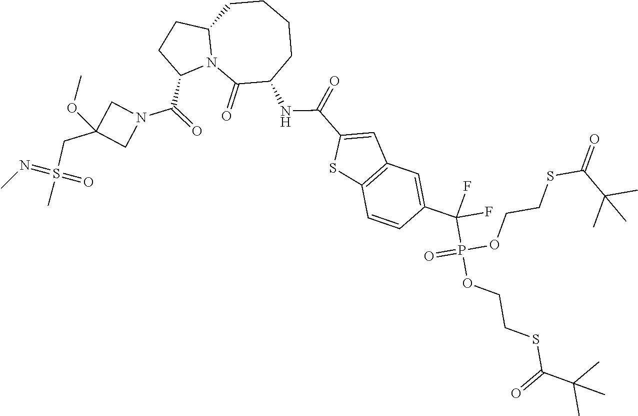 [SMILES: CN=S(C)(=O)CC1(OC)CN(C(=O)[C@@H]2CC[C@@H]3CCCC[C@H](NC(=O)c4cc5cc(C(F)(F)P(=O)(OCCSC(=O)C(C)(C)C)OCCSC(=O)C(C)(C)C)ccc5s4)C(=O)N32)C1] |
| 1016 | (R)-sec-butyl (((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)(phenoxy) phosphoryl)-L- alaninate | 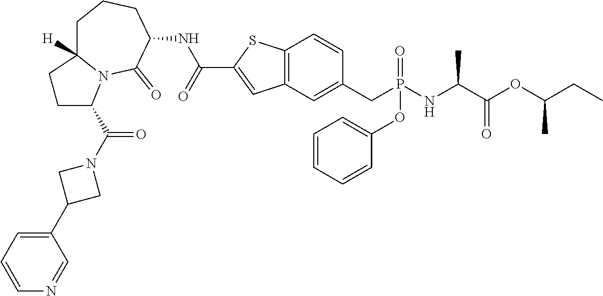 [SMILES: CC[C@@H](C)OC(=O)[C@H](C)NP(=O)(Cc1ccc2sc(C(=O)N[C@H]3CCC[C@H]4CC[C@@H](C(=O)N5CC(c6cccnc6)C5)N4C3=O)cc2c1)Oc1ccccc1] |
| 1017 | propyl N-(((2- (((3S,6S,9aS)-3-(3- (4- (dimethylamino) pyridin-3-yl) azetidine- 1-carbonyl)-5- oxooctahydro-1H- pyrrolo[1,2- a]azepin-6- yl)carbamoyl)benzo [b]thiophen-5- yl)methyl)(phenoxy) phosphoryl)-O- methyl-L-serinate | 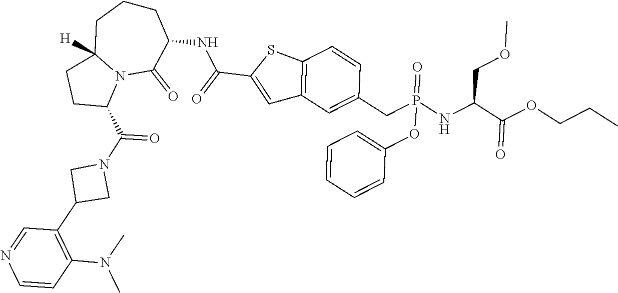 |
| 1018 | propyl (((S)- fluoro(2- (((3S,6S,9aS)-3-(3- (4-((S)-2- (methoxymethyl) azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 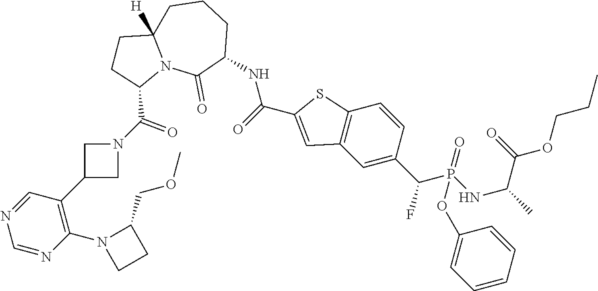 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cncnc6N6CC[C@H]6COC)C5)N4C3=O)cc2c1] |
| 1019 | propyl (((2- (((3S,6S,9aS)-3-(3- (4-(azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 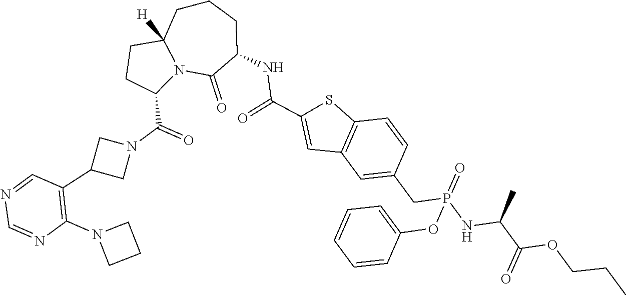 [SMILES: CCCOC(=O)[C@H](C)NP(=O)(Cc1ccc2sc(C(=O)N[C@H]3CCC[C@H]4CC[C@@H](C(=O)N5CC(c6cncnc6N6CCC6)C5)N4C3=O)cc2c1)Oc1ccccc1] |
| 1020 | propyl (((2- (((3S,6S,10aS)-3- (3-((2,2- difluoroethyl)amino)- 3-(pyridin-2- yl)azetidine-1- carbonyl)-5- oxodecahydropyrrolo [1,2-a]azocin-6- yl)carbamoyl)benzo [b]thiophen-5- yl)fluoromethyl) (phenoxy) phosphoryl)- L-alaninate | 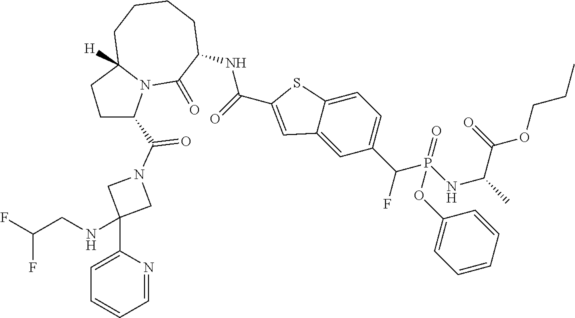 [SMILES: CCCOC(=O)[C@H](C)NP(=O)(Oc1ccccc1)C(F)c1ccc2sc(C(=O)N[C@H]3CCCC[C@H]4CC[C@@H](C(=O)N5CC(NCC(F)F)(c6ccccn6)C5)N4C3=O)cc2c1] |
| 1021 | propyl (((2- (((3S,6S,8R,9aR)- 3-(3-(4- (dimethylamino) pyridin-3- yl)azetidine- 1-carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 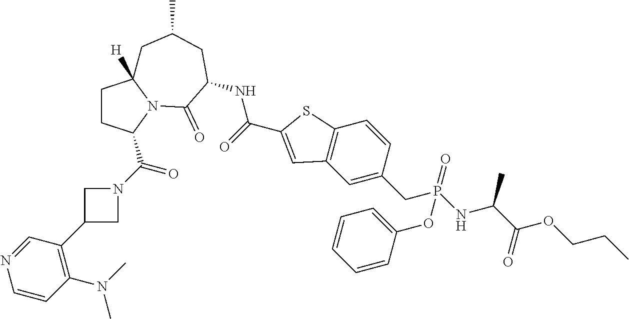 [SMILES: CCCOC(=O)[C@H](C)NP(=O)(Cc1ccc2sc(C(=O)N[C@H]3C[C@@H](C)C[C@H]4CC[C@@H](C(=O)N5CC(c6cnccc6N(C)C)C5)N4C3=O)cc2c1)Oc1ccccc1] |
| 1022 | propyl (((R)-(2- (((3S,6S,9aS)- 3-(3-(4- (dimethylamino)-5- fluoropyridin-3- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy) phosphoryl)- L-alaninate | 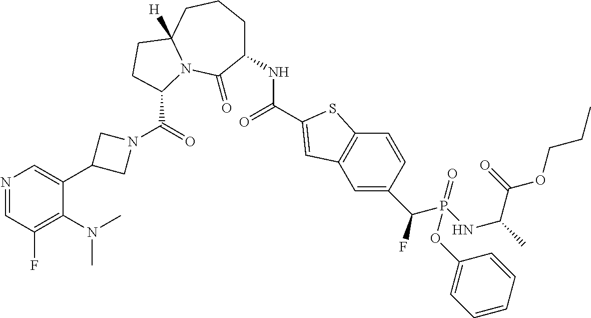 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cncc(F)c6N(C)C)C5)N4C3=O)cc2c1] |
| 1023 | isopropyl ((S)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 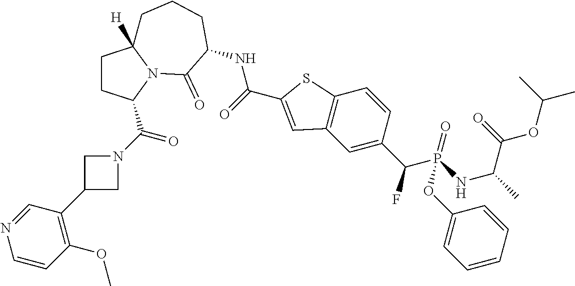 [SMILES: COc1ccncc1C1CN(C(=O)[C@@H]2CC[C@@H]3CCC[C@H](NC(=O)c4cc5cc([C@H](F)[P@@](=O)(N[C@@H](C)C(=O)OC(C)C)Oc6ccccc6)ccc5s4)C(=O)N32)C1] |
| | or | or |
| | isopropyl ((R)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 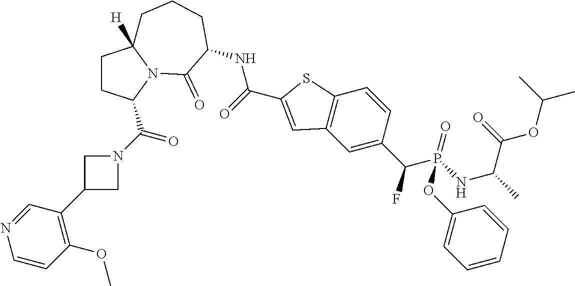 [SMILES: COc1ccncc1C1CN(C(=O)[C@@H]2CC[C@@H]3CCC[C@H](NC(=O)c4cc5cc([C@H](F)[P@](=O)(N[C@@H](C)C(=O)OC(C)C)Oc6ccccc6)ccc5s4)C(=O)N32)C1] |
| | | Less polar linker building block |
| | | was used in synthesis and |
| | | biological testing |
| 1024 | propyl (((S)-(2- (((3S,6S,9aS)-3-(3- (4-(azetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy) phosphoryl)- L-alaninate | 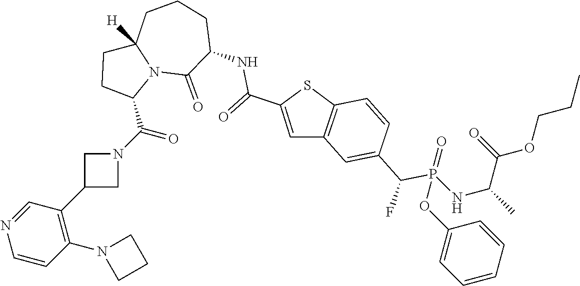 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cnccc6N6CCC6)C5)N4C3=O)cc2c1] |
| 1025 | propyl (((2- (((3S,6S,9aS)-3-(3- (4- (dimethylamino) pyridin-3- yl)azetidine- 1-carbonyl)-5- oxooctahydro-1H- pyrrolo[1,2- a]azepin-6- yl)carbamoyl)benzo [b]thiophen-5- yl)methyl)(2,2,2- trifluoroethoxy) phosphoryl)-L- alaninate | 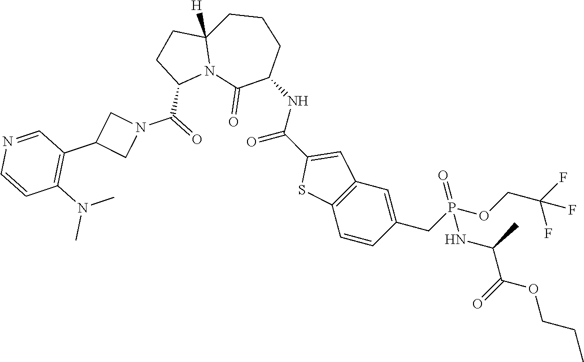 [SMILES: CCCOC(=O)[C@H](C)NP(=O)(Cc1ccc2sc(C(=O)N[C@H]3CCC[C@H]4CC[C@@H](C(=O)N5CC(c6cnccc6N(C)C)C5)N4C3=O)cc2c1)OCC(F)(F)F] |
| 1026 | propyl (((R)- fluoro(2- (((3S,6S,9aS)-3-(3- (5-fluoro-4- (methylamino) pyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 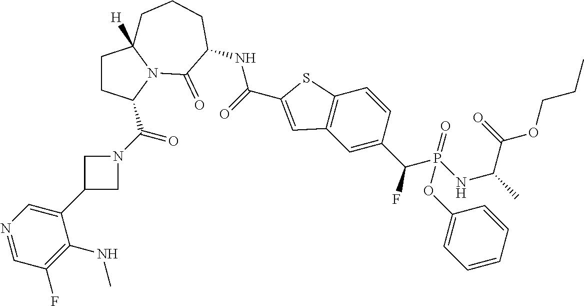 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cncc(F)c6NC)C5)N4C3=O)cc2c1] |
| 1027 | propyl ((S)-((R)- fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 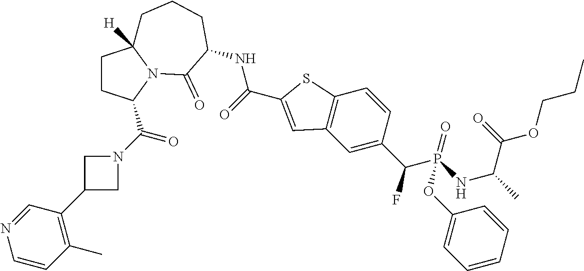 [SMILES: CCCOC(=O)[C@H](C)N[P@@](=O)(Oc1ccccc1)[C@@H](F)c1ccc2sc(C(=O)N[C@H]3CCC[C@H]4CC[C@@H](C(=O)N5CC(c6cnccc6C)C5)N4C3=O)cc2c1] |
| | or | or |
| | propyl ((R)-((R)- fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 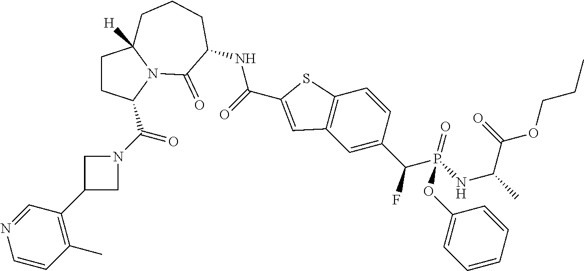 [SMILES: CCCOC(=O)[C@H](C)N[P@](=O)(Oc1ccccc1)[C@@H](F)c1ccc2sc(C(=O)N[C@H]3CCC[C@H]4CC[C@@H](C(=O)N5CC(c6cnccc6C)C5)N4C3=O)cc2c1] |
| | | CFTE-28A, SFC peak 2 was |
| | | used in synthesis and biological testing |
| 1028 | propyl (((R)-(2- (((3S,6S,9aS)-3-(3- (4-(azetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy) phosphoryl)- L-alaninate | 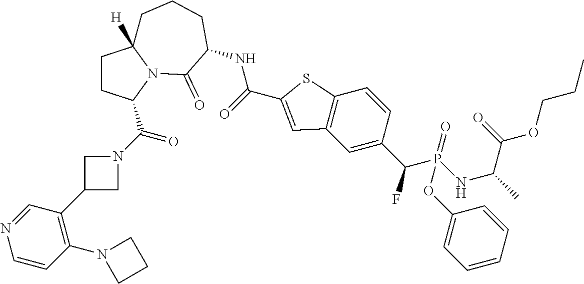 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cnccc6N6CCC6)C5)N4C3=O)cc2c1] |
| 1029 | propyl (((R)-(2- (((3S,6S,8R,9aR)- 3-(3-(4- (dimethylamino) pyridin-3-yl) azetidine- 1-carbonyl)-8- methyl-5- oxooctahydro-1H- pyrrolo[1,2- a]azepin-6- yl)carbamoyl)benzo [b]thiophen-5- yl)fluoromethyl) (phenoxy) phosphoryl)- L-alaninate | 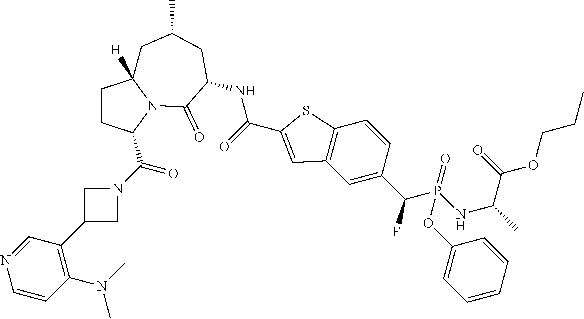 [SMILES: CCCOC(=O)[C@H](C)NP(=O)(Oc1ccccc1)[C@@H](F)c1ccc2sc(C(=O)N[C@H]3C[C@@H](C)C[C@H]4CC[C@@H](C(=O)N5CC(c6cnccc6N(C)C)C5)N4C3=O)cc2c1] |
| 1030 | propyl (((R)- fluoro(2- (((3S,6S,9aS)-3- (((3S,4S)-4-fluoro- 1-(1H-imidazol-2- yl)pyrrolidin-3- yl)carbamoyl)-5- oxooctahydro-1H- pyrrolo[1,2- a]azepin-6- yl)carbamoyl)benzo [b]thiophen-5- yl)methyl)(phenoxy) phosphoryl)-L- alaninate | 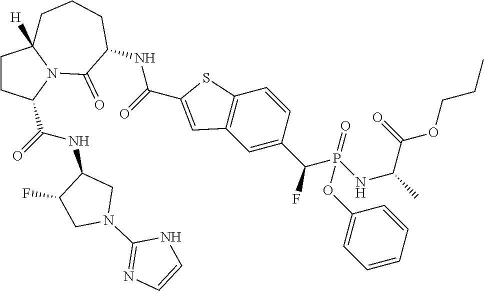 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N[C@H]5CN(c6ncc[nH]6)C[C@@H]5F)N4C3=O)cc2c1] |
| 1031 | propyl (((2- (((3S,6S,9aS)-3-(3- (4-(azetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 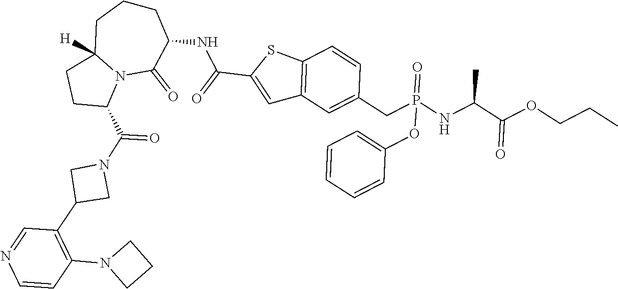 |
| 1032 | propyl ((S)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate or | 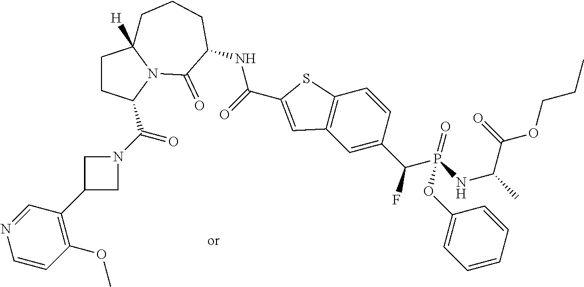 [SMILES: CCCOC(=O)[C@H](C)N[P@@](=O)(Oc1ccccc1)[C@@H](F)c1ccc2sc(C(=O)N[C@H]3CCC[C@H]4CC[C@@H](C(=O)N5CC(c6cnccc6OC)C5)N4C3=O)cc2c1] |
| | propyl ((R)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) )phosphoryl)-L- alaninate | 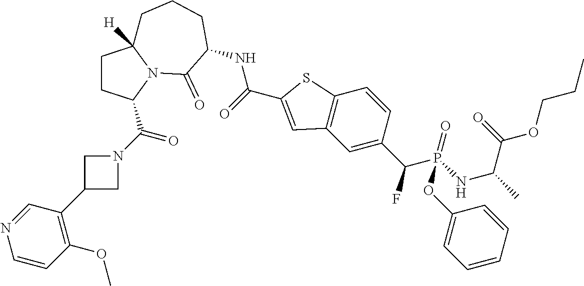 [SMILES: CCCOC(=O)[C@H](C)N[P@](=O)(Oc1ccccc1)[C@@H](F)c1ccc2sc(C(=O)N[C@H]3CCC[C@H]4CC[C@@H](C(=O)N5CC(c6cnccc6OC)C5)N4C3=O)cc2c1] |
| | | CFTE-28B, SFC peak 1 was |
| | | used for synthesis and biological |
| | | testing |
| 1033 | propyl ((R)-((R)- fluoro(2- (((3S,6S,8R,9aR)- 3-(3-(4- isopropoxypyridin- 3-yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 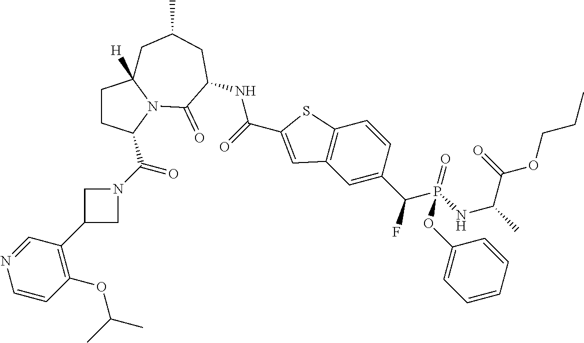 [SMILES: CCCOC(=O)[C@H](C)N[P@](=O)(Oc1ccccc1)[C@@H](F)c1ccc2sc(C(=O)N[C@H]3C[C@@H](C)C[C@H]4CC[C@@H](C(=O)N5CC(c6cnccc6OC(C)C)C5)N4C3=O)cc2c1] |
| | or | or |
| | propyl ((S)-((R)- fluoro(2- (((3S,6S,8R,9aR)- 3-(3-(4- isopropoxypyridin- 3-yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 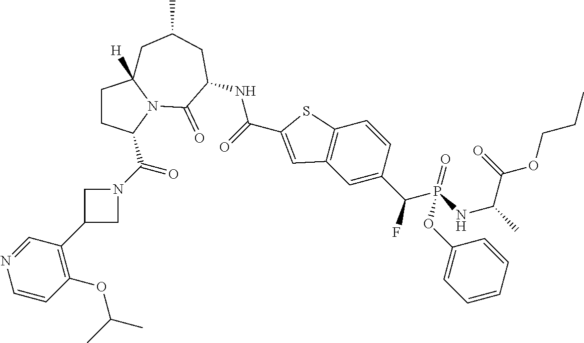 [SMILES: CCCOC(=O)[C@H](C)N[P@@](=O)(Oc1ccccc1)[C@@H](F)c1ccc2sc(C(=O)N[C@H]3C[C@@H](C)C[C@H]4CC[C@@H](C(=O)N5CC(c6cnccc6OC(C)C)C5)N4C3=O)cc2c1] |
| | | CFTE-28B, SFC peak 1 was |
| | | used for synthesis and biological |
| | | testing |
| 1034 | propyl ((R)-((R)- fluoro(2- (((3S,6S,8R,9aR)- 3-(3-(4- isopropoxypyridin- 3-yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 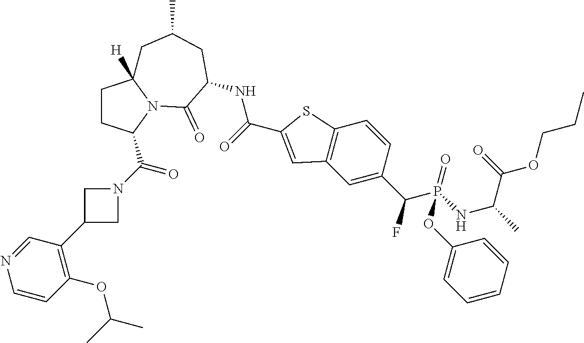 [SMILES: CCCOC(=O)[C@H](C)N[P@](=O)(Oc1ccccc1)[C@@H](F)c1ccc2sc(C(=O)N[C@H]3C[C@@H](C)C[C@H]4CC[C@@H](C(=O)N5CC(c6cnccc6OC(C)C)C5)N4C3=O)cc2c1] |
| | or | or |
| | propyl ((S)-((R)- fluoro(2- (((3S,6S,8R,9aR)- 3-(3-(4- isopropoxypyridin- 3-yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 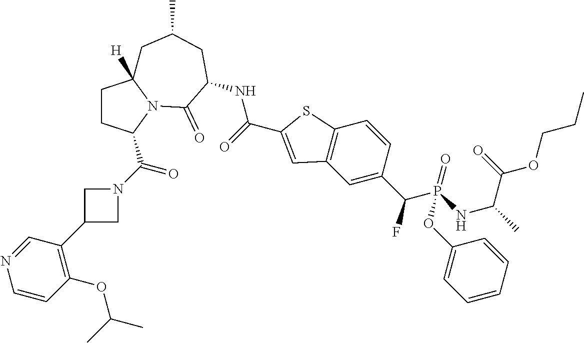 [SMILES: CCCOC(=O)[C@H](C)N[P@@](=O)(Oc1ccccc1)[C@@H](F)c1ccc2sc(C(=O)N[C@H]3C[C@@H](C)C[C@H]4CC[C@@H](C(=O)N5CC(c6cnccc6OC(C)C)C5)N4C3=O)cc2c1] |
| | | CFTE-28A, SFC peak 2 was |
| | | used for synthesis and biological |
| | | testing |
| 1035 | propyl ((R)-((R)- fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 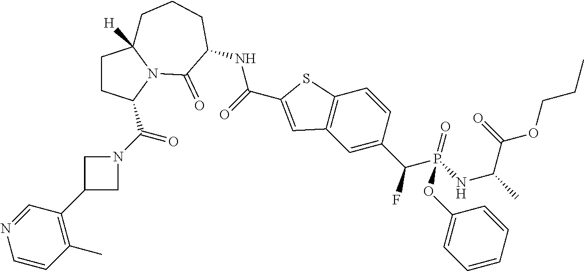 [SMILES: CCCOC(=O)[C@H](C)N[P@](=O)(Oc1ccccc1)[C@@H](F)c1ccc2sc(C(=O)N[C@H]3CCC[C@H]4CC[C@@H](C(=O)N5CC(c6cnccc6C)C5)N4C3=O)cc2c1] |
| | or | or |
| | propyl ((S)-((R)- fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 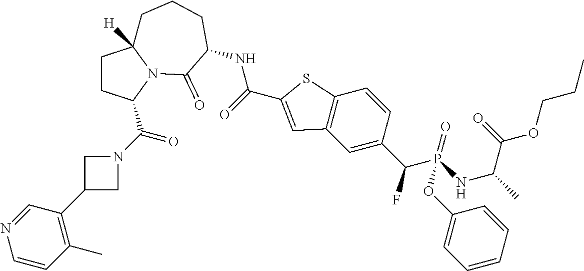 [SMILES: CCCOC(=O)[C@H](C)N[P@@](=O)(Oc1ccccc1)[C@@H](F)c1ccc2sc(C(=O)N[C@H]3CCC[C@H]4CC[C@@H](C(=O)N5CC(c6cnccc6C)C5)N4C3=O)cc2c1] |
| | | CFTE-28B, SFC peak 1 was |
| | | used for synthesis and biological |
| | | testing |
| 1036 | propyl ((S)-((R)- fluoro(2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 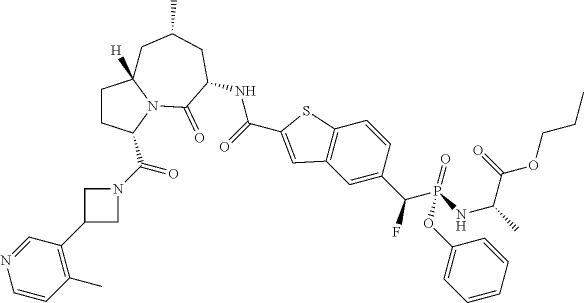 [SMILES: CCCOC(=O)[C@H](C)N[P@@](=O)(Oc1ccccc1)[C@@H](F)c1ccc2sc(C(=O)N[C@H]3C[C@@H](C)C[C@H]4CC[C@@H](C(=O)N5CC(c6cnccc6C)C5)N4C3=O)cc2c1] |
| | or | or |
| | propyl ((R)-((R)- fluoro(2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 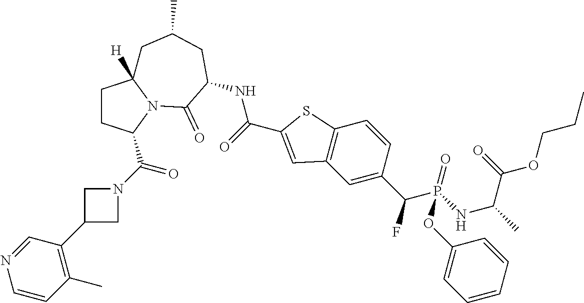 [SMILES: CCCOC(=O)[C@H](C)N[P@](=O)(Oc1ccccc1)[C@@H](F)c1ccc2sc(C(=O)N[C@H]3C[C@@H](C)C[C@H]4CC[C@@H](C(=O)N5CC(c6cnccc6C)C5)N4C3=O)cc2c1] |
| | | CFTE-28B, SFC peak 1 was |
| | | used for synthesis and biological |
| | | testing |
| 1037 | propyl ((S)-((R)- fluoro(2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 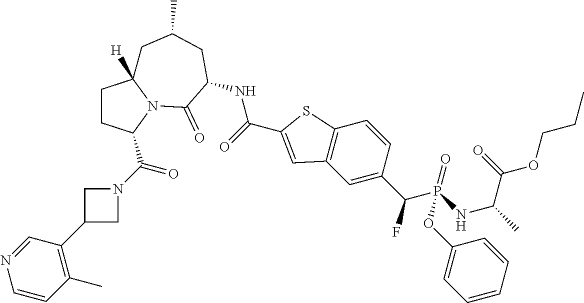 [SMILES: CCCOC(=O)[C@H](C)N[P@@](=O)(Oc1ccccc1)[C@@H](F)c1ccc2sc(C(=O)N[C@H]3C[C@@H](C)C[C@H]4CC[C@@H](C(=O)N5CC(c6cnccc6C)C5)N4C3=O)cc2c1] |
| | or | or |
| | propyl ((R)-((R)- fluoro(2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 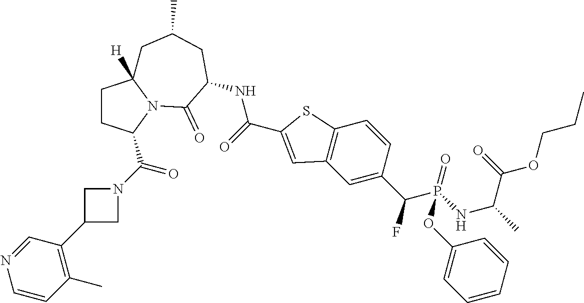 [SMILES: CCCOC(=O)[C@H](C)N[P@](=O)(Oc1ccccc1)[C@@H](F)c1ccc2sc(C(=O)N[C@H]3C[C@@H](C)C[C@H]4CC[C@@H](C(=O)N5CC(c6cnccc6C)C5)N4C3=O)cc2c1] |
| | | CFTE-28A, SFC peak 2 was |
| | | used for synthesis and biological |
| | | testing |
| 1038 | propyl (((2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 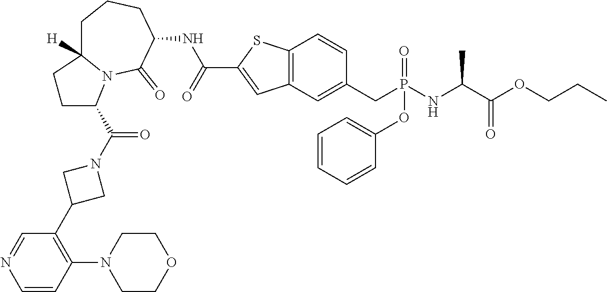 [SMILES: CCCOC(=O)[C@H](C)NP(=O)(Cc1ccc2sc(C(=O)N[C@H]3CCC[C@H]4CC[C@@H](C(=O)N5CC(c6cnccc6N6CCOCC6)C5)N4C3=O)cc2c1)Oc1ccccc1] |
| 1039 | propyl ((S)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 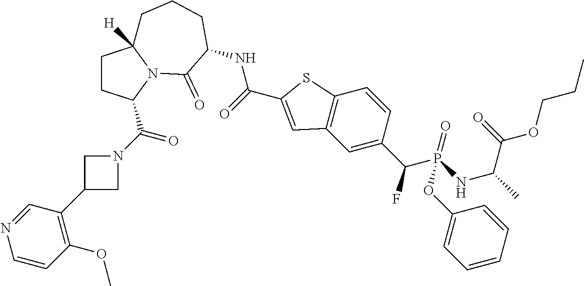 [SMILES: CCCOC(=O)[C@H](C)N[P@@](=O)(Oc1ccccc1)[C@@H](F)c1ccc2sc(C(=O)N[C@H]3CCC[C@H]4CC[C@@H](C(=O)N5CC(c6cnccc6OC)C5)N4C3=O)cc2c1] |
| | or | or |
| | propyl ((R)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 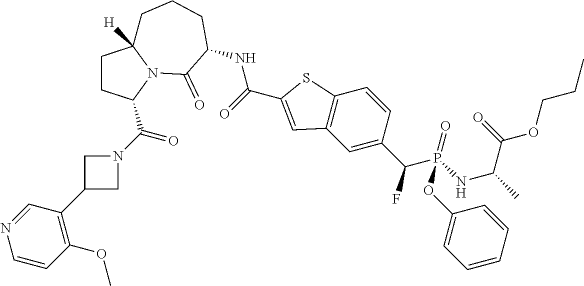 [SMILES: CCCOC(=O)[C@H](C)N[P@](=O)(Oc1ccccc1)[C@@H](F)c1ccc2sc(C(=O)N[C@H]3CCC[C@H]4CC[C@@H](C(=O)N5CC(c6cnccc6OC)C5)N4C3=O)cc2c1] |
| | | CFTE-28A, SFC peak 2 was |
| | | used synthesis and biological |
| | | testing |
| 1040 | propyl ((S)-((R)- fluoro(2- (((3S,6S,8S,9aR)-3- (3-(4- methoxypyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 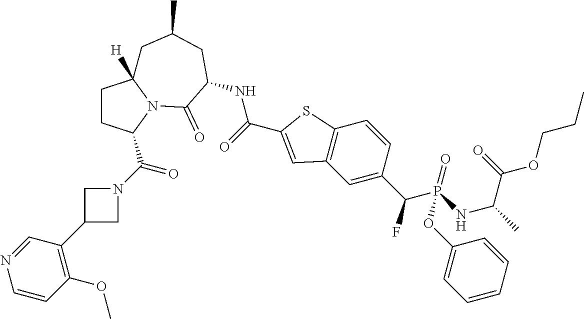 [SMILES: CCCOC(=O)[C@H](C)N[P@@](=O)(Oc1ccccc1)[C@@H](F)c1ccc2sc(C(=O)N[C@H]3C[C@H](C)C[C@H]4CC[C@@H](C(=O)N5CC(c6cnccc6OC)C5)N4C3=O)cc2c1] |
| | or | or |
| | propyl ((R)-((R)- fluoro(2- (((3S,6S,8S,9aR)-3- (3-(4- methoxypyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 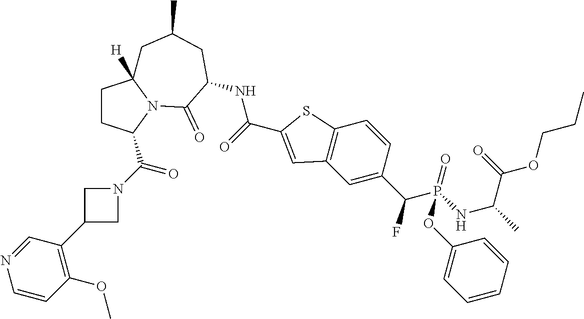 [SMILES: CCCOC(=O)[C@H](C)N[P@](=O)(Oc1ccccc1)[C@@H](F)c1ccc2sc(C(=O)N[C@H]3C[C@H](C)C[C@H]4CC[C@@H](C(=O)N5CC(c6cnccc6OC)C5)N4C3=O)cc2c1] |
| | | CFTE-28B, SFC peak 1 was |
| | | used for synthesis and biological |
| | | testing |
| 1041 | propyl ((S)-((R)- fluoro(2- (((3S,6S,8S,9aR)-3- (3-(4- methoxypyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 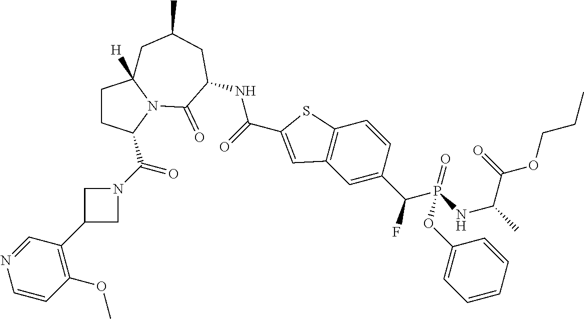 [SMILES: CCCOC(=O)[C@H](C)N[P@@](=O)(Oc1ccccc1)[C@@H](F)c1ccc2sc(C(=O)N[C@H]3C[C@H](C)C[C@H]4CC[C@@H](C(=O)N5CC(c6cnccc6OC)C5)N4C3=O)cc2c1] |
| | or | or |
| | propyl ((R)-((R)- fluoro(2- (((3S,6S,8S,9aR)-3- (3-(4- methoxypyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 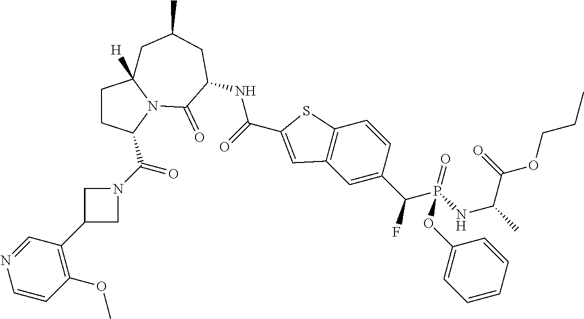 [SMILES: CCCOC(=O)[C@H](C)N[P@](=O)(Oc1ccccc1)[C@@H](F)c1ccc2sc(C(=O)N[C@H]3C[C@H](C)C[C@H]4CC[C@@H](C(=O)N5CC(c6cnccc6OC)C5)N4C3=O)cc2c1] |
| | | CFTE-28A, SFC peak 2 was |
| | | used synthesis and biological |
| | | testing |
| 1042 | propyl ((fluoro(2- (((3S,6S,9aS)-3-(3- (4- methoxypyrimidin- 5-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 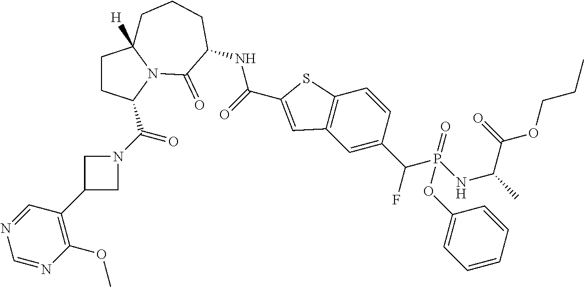 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cncnc6OC)C5)N4C3=O)cc2c1] |
| 1043 | propyl (((2- (((3S,6S,9aS)-3-(3- (4- (dimethylamino) pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) (phenoxyphosphoryl)- L-alaninate | 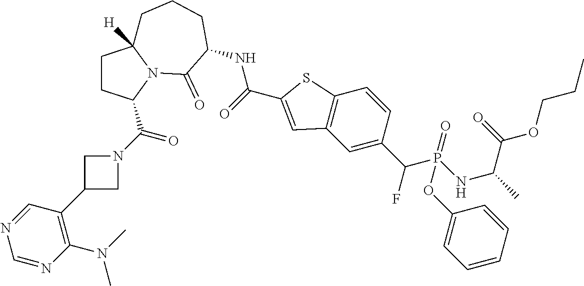 [SMILES: CCCOC(=O)[C@H](C)NP(=O)(Oc1ccccc1)C(F)c1ccc2sc(C(=O)N[C@H]3CCC[C@H]4CC[C@@H](C(=O)N5CC(c6cncnc6N(C)C)C5)N4C3=O)cc2c1] |
| 1044 | propyl ((R)-((S)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 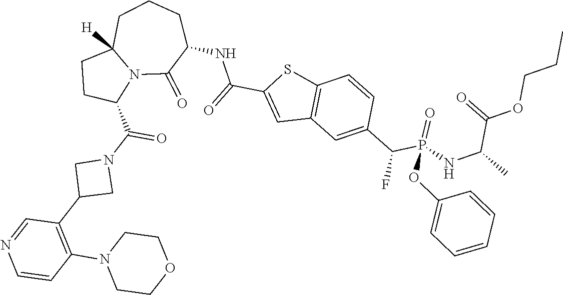 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cnccc6N6CCOCC6)C5)N4C3=O)cc2c1] |
| | or | or |
| | propyl ((S)-((S)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 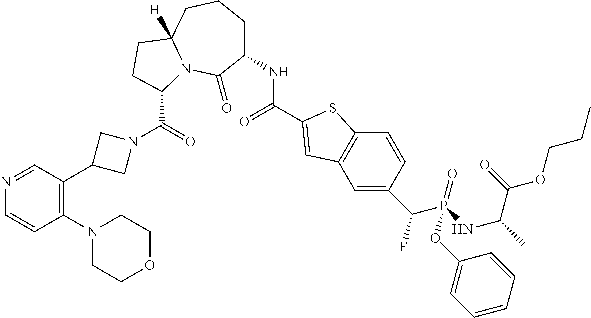 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cnccc6N6CCOCC6)C5)N4C3=O)cc2c1] |
| | | CFTE-27B, SFC peak 2 was |
| | | used synthesis and biological |
| | | testing |
| 1045 | propyl ((R)-((S)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 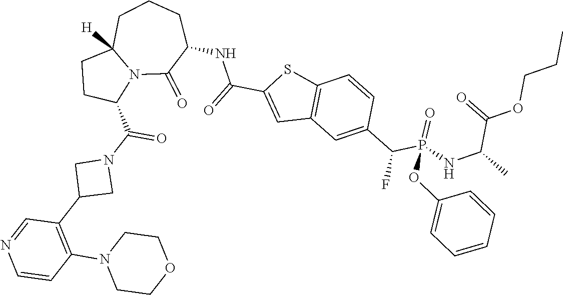 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cnccc6N6CCOCC6)C5)N4C3=O)cc2c1] |
| | or | or |
| | propyl ((S)-((S)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 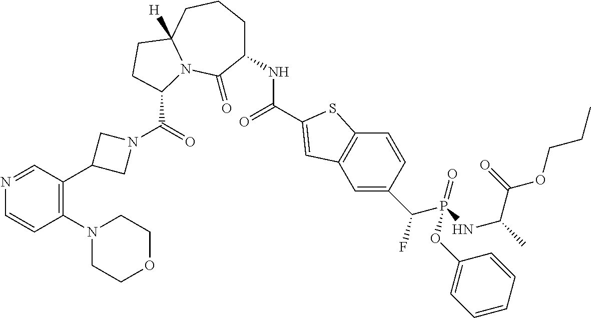 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cnccc6N6CCOCC6)C5)N4C3=O)cc2c1] |
| | | CFTE-27A, SFC peak 1 was |
| | | used synthesis and biological |
| | | testing |
| 1046 | propyl ((R)-((R)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 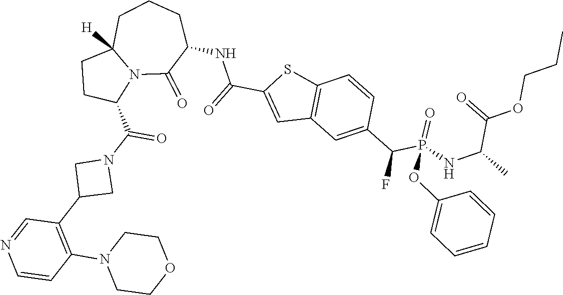 [SMILES: CCCOC(=O)[C@H](C)N[P@](=O)(Oc1ccccc1)[C@@H](F)c1ccc2sc(C(=O)N[C@H]3CCC[C@H]4CC[C@@H](C(=O)N5CC(c6cnccc6N6CCOCC6)C5)N4C3=O)cc2c1] |
| | or | or |
| | propyl ((S)-((R)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 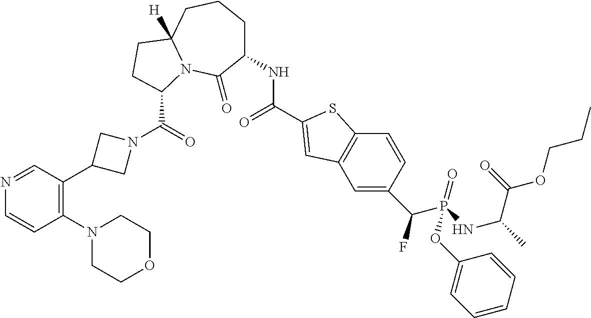 [SMILES: CCCOC(=O)[C@H](C)N[P@@](=O)(Oc1ccccc1)[C@@H](F)c1ccc2sc(C(=O)N[C@H]3CCC[C@H]4CC[C@@H](C(=O)N5CC(c6cnccc6N6CCOCC6)C5)N4C3=O)cc2c1] |
| | | CFTE-28B, SFC peak1 was used |
| | | for synthesis and biological |
| | | testing |
| 1047 | propyl ((R)-((R)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 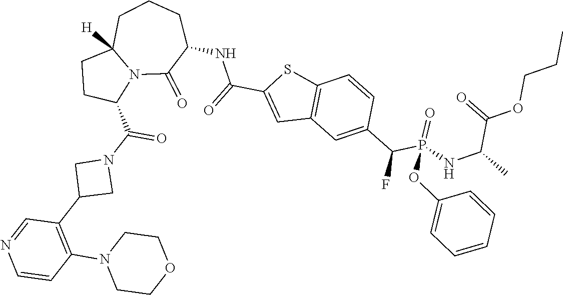 [SMILES: CCCOC(=O)[C@H](C)N[P@](=O)(Oc1ccccc1)[C@@H](F)c1ccc2sc(C(=O)N[C@H]3CCC[C@H]4CC[C@@H](C(=O)N5CC(c6cnccc6N6CCOCC6)C5)N4C3=O)cc2c1] |
| | or | or |
| | propyl ((S)-((R)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 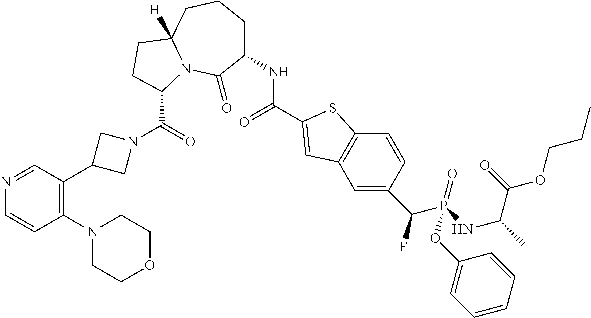 [SMILES: CCCOC(=O)[C@H](C)N[P@@](=O)(Oc1ccccc1)[C@@H](F)c1ccc2sc(C(=O)N[C@H]3CCC[C@H]4CC[C@@H](C(=O)N5CC(c6cnccc6N6CCOCC6)C5)N4C3=O)cc2c1] |
| | | CFTE-28A, SFC peak 2 was |
| | | used for synthesis and biological |
| | | testing |
| 1048 | propyl ((R)-((S)- fluoro(2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 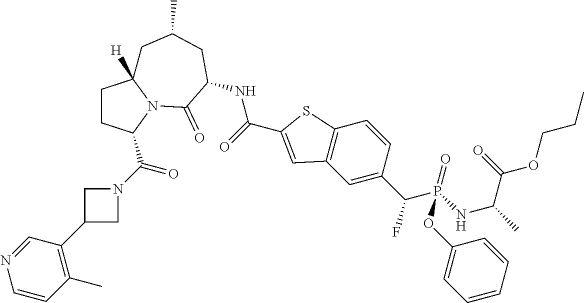 [SMILES: CCCOC(=O)[C@H](C)N[P@](=O)(Oc1ccccc1)[C@H](F)c1ccc2sc(C(=O)N[C@H]3C[C@@H](C)C[C@H]4CC[C@@H](C(=O)N5CC(c6cnccc6C)C5)N4C3=O)cc2c1] |
| | or | or |
| | propyl ((S)-((S)- fluoro(2- (((3S,6S,8R,9aR)- 8-methyl-3-(3-(4- methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 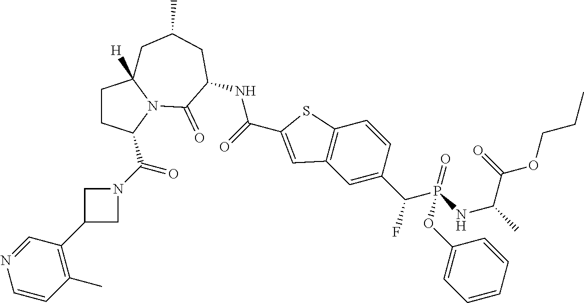 [SMILES: CCCOC(=O)[C@H](C)N[P@@](=O)(Oc1ccccc1)[C@H](F)c1ccc2sc(C(=O)N[C@H]3C[C@@H](C)C[C@H]4CC[C@@H](C(=O)N5CC(c6cnccc6C)C5)N4C3=O)cc2c1] |
| | | CFTE-27A, SFC peak 1 was |
| | | used for synthesis and biological |
| | | testing |
| 1049 | propyl ((S)-((S)- fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate or | 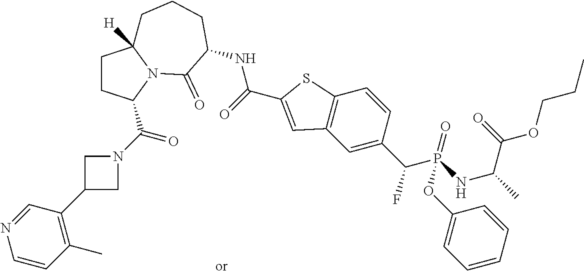 [SMILES: CCCOC(=O)[C@H](C)N[P@@](=O)(Oc1ccccc1)[C@H](F)c1ccc2sc(C(=O)N[C@H]3CCC[C@H]4CC[C@@H](C(=O)N5CC(c6cnccc6C)C5)N4C3=O)cc2c1] |
| | propyl ((R)-((S)- fluoro(2- (((3S,6S,9aS)-3-(3- (4-methylpyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 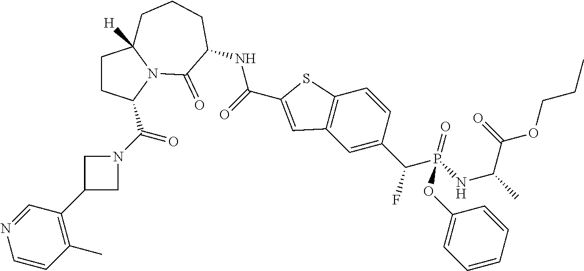 [SMILES: CCCOC(=O)[C@H](C)N[P@](=O)(Oc1ccccc1)[C@H](F)c1ccc2sc(C(=O)N[C@H]3CCC[C@H]4CC[C@@H](C(=O)N5CC(c6cnccc6C)C5)N4C3=O)cc2c1] |
| | | CFTE-27A, SFC peak 1 was |
| | | used for synthesis and biological |
| | | testing |
| 1050 | benzyl ((S)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 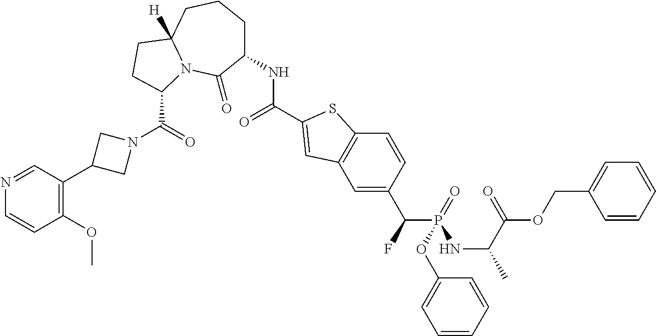 [SMILES: COc1ccncc1C1CN(C(=O)[C@@H]2CC[C@@H]3CCC[C@H](NC(=O)c4cc5cc([C@H](F)[P@@](=O)(N[C@@H](C)C(=O)OCc6ccccc6)Oc6ccccc6)ccc5s4)C(=O)N32)C1] |
| | or | or |
| | benzyl ((R)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 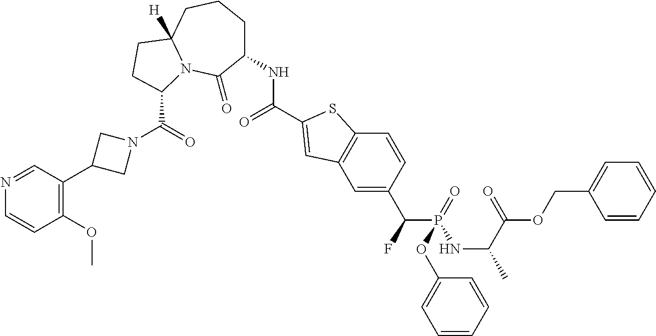 [SMILES: COc1ccncc1C1CN(C(=O)[C@@H]2CC[C@@H]3CCC[C@H](NC(=O)c4cc5cc([C@H](F)[P@](=O)(N[C@@H](C)C(=O)OCc6ccccc6)Oc6ccccc6)ccc5s4)C(=O)N32)C1] |
| | | More polar linker building block |
| | | was used synthesis and |
| | | biological testing |
| 1051 | benzyl ((S)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate or | 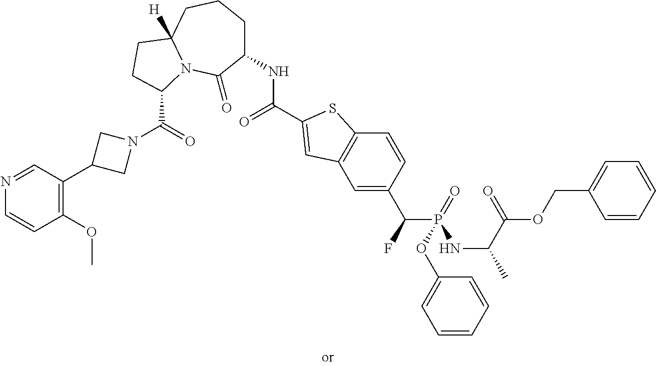 [SMILES: COc1ccncc1C1CN(C(=O)[C@@H]2CC[C@@H]3CCC[C@H](NC(=O)c4cc5cc([C@H](F)[P@@](=O)(N[C@@H](C)C(=O)OCc6ccccc6)Oc6ccccc6)ccc5s4)C(=O)N32)C1] |
| | benzyl ((R)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 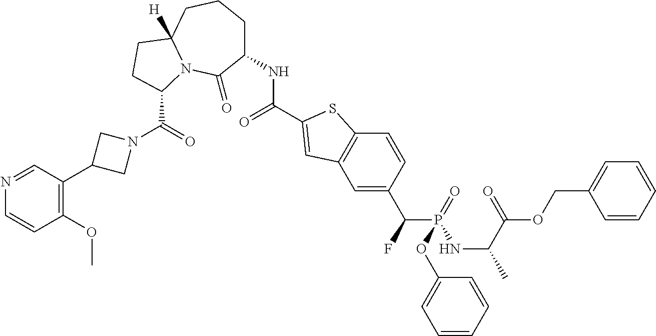 [SMILES: COc1ccncc1C1CN(C(=O)[C@@H]2CC[C@@H]3CCC[C@H](NC(=O)c4cc5cc([C@H](F)[P@](=O)(N[C@@H](C)C(=O)OCc6ccccc6)Oc6ccccc6)ccc5s4)C(=O)N32)C1] |
| | | Less polar linker building block |
| | | was used in synthesis and |
| | | biological testing |
| 1052 | isopropyl ((S)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 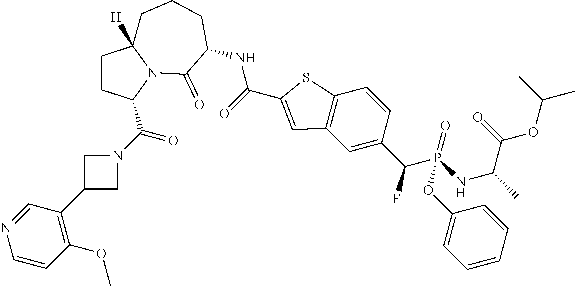 [SMILES: COc1ccncc1C1CN(C(=O)[C@@H]2CC[C@@H]3CCC[C@H](NC(=O)c4cc5cc([C@H](F)[P@@](=O)(N[C@@H](C)C(=O)OC(C)C)Oc6ccccc6)ccc5s4)C(=O)N32)C1] |
| | or | or |
| | isopropyl ((R)-((R)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 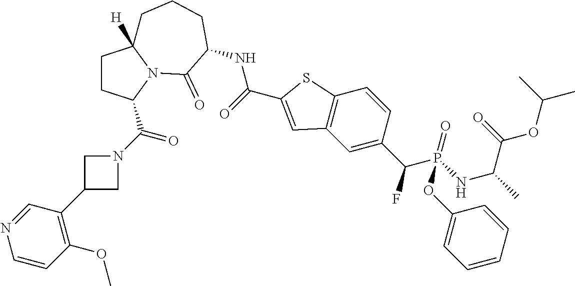 [SMILES: COc1ccncc1C1CN(C(=O)[C@@H]2CC[C@@H]3CCC[C@H](NC(=O)c4cc5cc([C@H](F)[P@](=O)(N[C@@H](C)C(=O)OC(C)C)Oc6ccccc6)ccc5s4)C(=O)N32)C1] |
| | | More polar linker building block |
| | | was used in synthesis and |
| | | biological testing |
| 1053 | isopropyl ((S)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 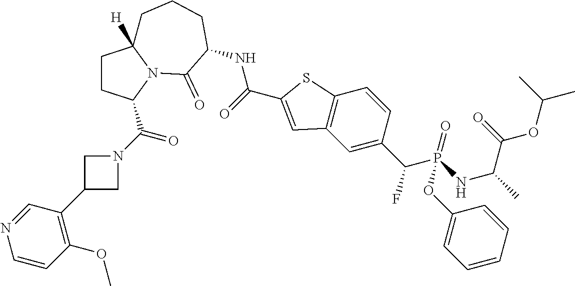 [SMILES: COc1ccncc1C1CN(C(=O)[C@@H]2CC[C@@H]3CCC[C@H](NC(=O)c4cc5cc([C@@H](F)[P@@](=O)(N[C@@H](C)C(=O)OC(C)C)Oc6ccccc6)ccc5s4)C(=O)N32)C1] |
| | or | or |
| | isopropyl ((R)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 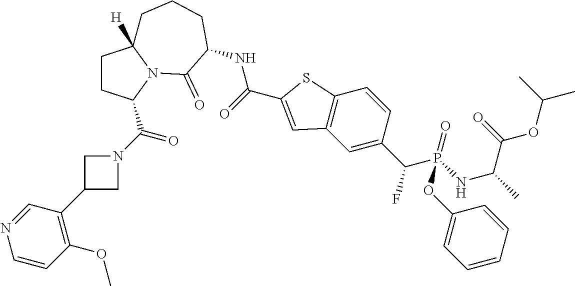 [SMILES: COc1ccncc1C1CN(C(=O)[C@@H]2CC[C@@H]3CCC[C@H](NC(=O)c4cc5cc([C@@H](F)[P@](=O)(N[C@@H](C)C(=O)OC(C)C)Oc6ccccc6)ccc5s4)C(=O)N32)C1] |
| | | Less polar linker building block |
| | | was used in synthesis and |
| | | biological testing |
| 1054 | benzyl ((R)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 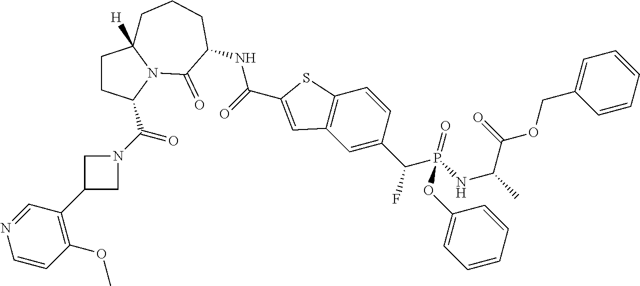 [SMILES: COc1ccncc1C1CN(C(=O)[C@@H]2CC[C@@H]3CCC[C@H](NC(=O)c4cc5cc([C@@H](F)[P@](=O)(N[C@@H](C)C(=O)OCc6ccccc6)Oc6ccccc6)ccc5s4)C(=O)N32)C1] |
| | or | or |
| | benzyl ((S)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 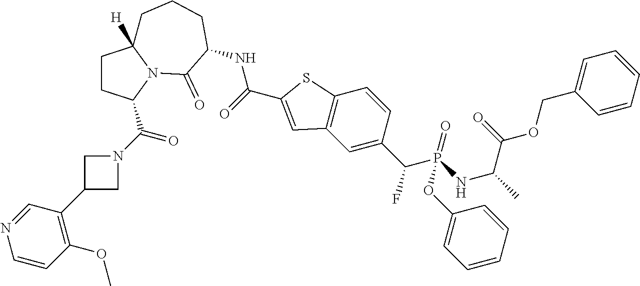 [SMILES: COc1ccncc1C1CN(C(=O)[C@@H]2CC[C@@H]3CCC[C@H](NC(=O)c4cc5cc([C@@H](F)[P@@](=O)(N[C@@H](C)C(=O)OCc6ccccc6)Oc6ccccc6)ccc5s4)C(=O)N32)C1] |
| | | More polar linker building block |
| | | was used in synthesis and |
| | | biological testing |
| 1055 | benzyl ((R)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 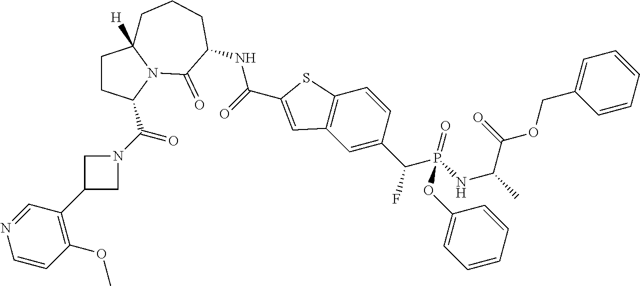 [SMILES: COc1ccncc1C1CN(C(=O)[C@@H]2CC[C@@H]3CCC[C@H](NC(=O)c4cc5cc([C@@H](F)[P@](=O)(N[C@@H](C)C(=O)OCc6ccccc6)Oc6ccccc6)ccc5s4)C(=O)N32)C1] |
| | or | or |
| | benzyl ((S)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 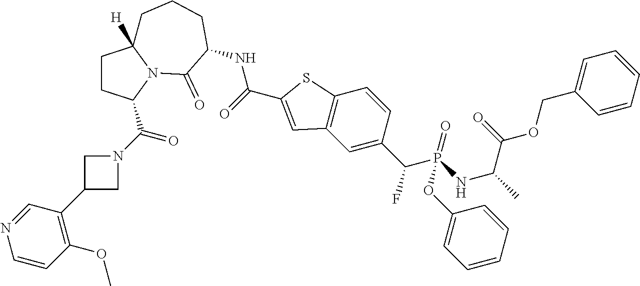 [SMILES: COc1ccncc1C1CN(C(=O)[C@@H]2CC[C@@H]3CCC[C@H](NC(=O)c4cc5cc([C@@H](F)[P@@](=O)(N[C@@H](C)C(=O)OCc6ccccc6)Oc6ccccc6)ccc5s4)C(=O)N32)C1] |
| | | Less polar linker building block |
| | | was used in synthesis and |
| | | biological testing |
| 1056 | neopentyl ((R)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 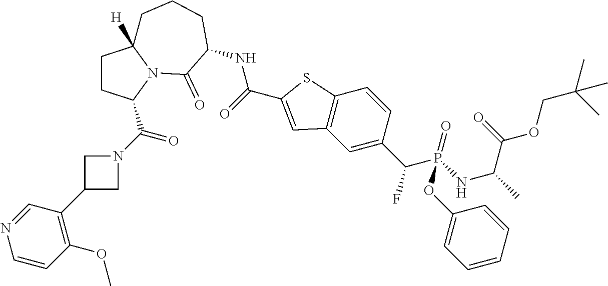 [SMILES: COc1ccncc1C1CN(C(=O)[C@@H]2CC[C@@H]3CCC[C@H](NC(=O)c4cc5cc([C@@H](F)[P@](=O)(N[C@@H](C)C(=O)OCC(C)(C)C)Oc6ccccc6)ccc5s4)C(=O)N32)C1] |
| | or | or |
| | neopentyl ((S)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 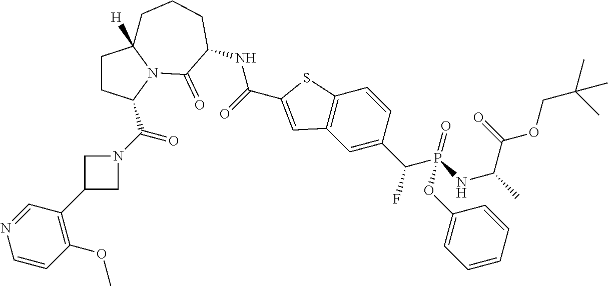 [SMILES: COc1ccncc1C1CN(C(=O)[C@@H]2CC[C@@H]3CCC[C@H](NC(=O)c4cc5cc([C@@H](F)[P@@](=O)(N[C@@H](C)C(=O)OCC(C)(C)C)Oc6ccccc6)ccc5s4)C(=O)N32)C1] |
| | | More polar linker building block |
| | | was used in synthesis and |
| | | biological testing |
| 1057 | neopentyl ((R)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate or | 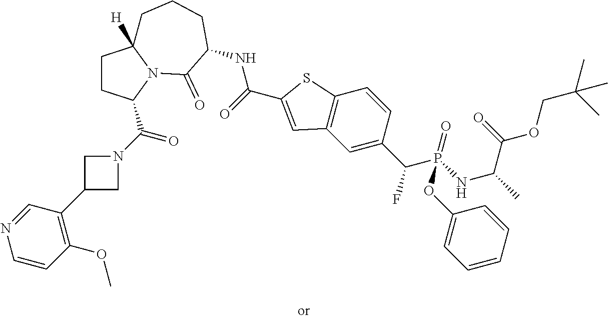 [SMILES: COc1ccncc1C1CN(C(=O)[C@@H]2CC[C@@H]3CCC[C@H](NC(=O)c4cc5cc([C@@H](F)[P@](=O)(N[C@@H](C)C(=O)OCC(C)(C)C)Oc6ccccc6)ccc5s4)C(=O)N32)C1] |
| | neopentyl ((S)-((S)- fluoro(2- (((3S,6S,9aS)-3-(3- (4-methoxypyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 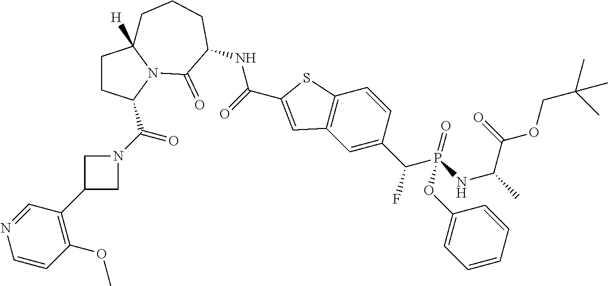 [SMILES: COc1ccncc1C1CN(C(=O)[C@@H]2CC[C@@H]3CCC[C@H](NC(=O)c4cc5cc([C@@H](F)[P@@](=O)(N[C@@H](C)C(=O)OCC(C)(C)C)Oc6ccccc6)ccc5s4)C(=O)N32)C1] |
| | | Less polar linker building block |
| | | was used in synthesis and |
| | | biological testing |
| 1058 | propyl ((R)-((R)- fluoro(2- (((3S,6S,8R,9aR)- 3-(3-(4- methoxypyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 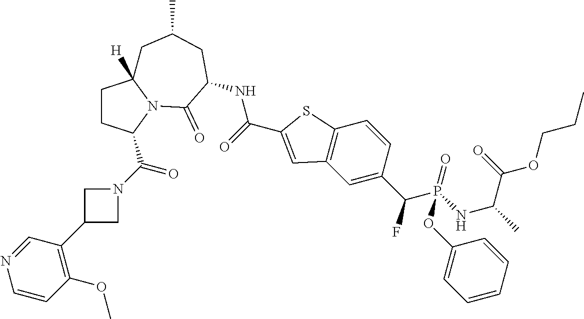 [SMILES: CCCOC(=O)[C@H](C)N[P@](=O)(Oc1ccccc1)[C@@H](F)c1ccc2sc(C(=O)N[C@H]3C[C@@H](C)C[C@H]4CC[C@@H](C(=O)N5CC(c6cnccc6OC)C5)N4C3=O)cc2c1] |
| | or | or |
| | propyl ((S)-((R)- fluoro(2- (((3S,6S,8R,9aR)- 3-(3-(4- methoxypyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 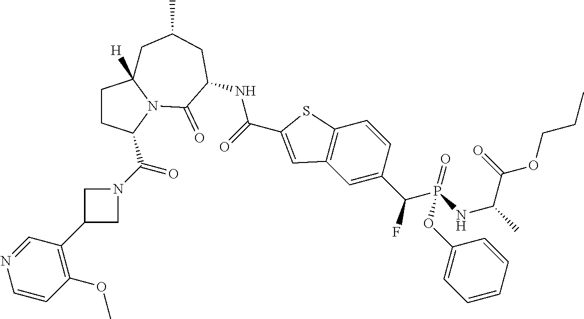 [SMILES: CCCOC(=O)[C@H](C)N[P@@](=O)(Oc1ccccc1)[C@@H](F)c1ccc2sc(C(=O)N[C@H]3C[C@@H](C)C[C@H]4CC[C@@H](C(=O)N5CC(c6cnccc6OC)C5)N4C3=O)cc2c1] |
| | | CFTE-28B, SFC peak 1 was |
| | | used in synthesis and biological testing |
| 1059 | propyl ((R)-((R)- fluoro(2- (((3S,6S,8R,9aR)- 3-(3-(4- methoxypyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 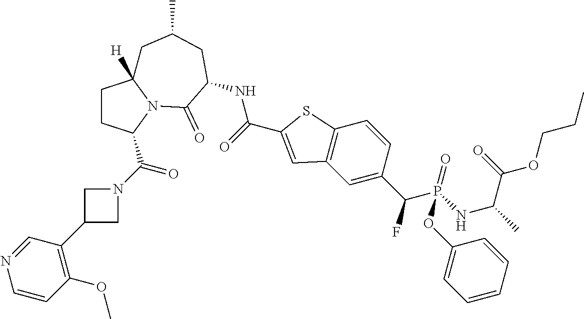 [SMILES: CCCOC(=O)[C@H](C)N[P@](=O)(Oc1ccccc1)[C@@H](F)c1ccc2sc(C(=O)N[C@H]3C[C@@H](C)C[C@H]4CC[C@@H](C(=O)N5CC(c6cnccc6OC)C5)N4C3=O)cc2c1] |
| | or | or |
| | propyl ((S)-((R)- fluoro(2- (((3S,6S,8R,9aR)- 3-(3-(4- methoxypyridin-3- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 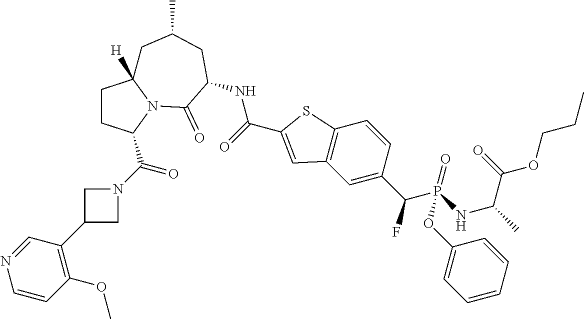 [SMILES: CCCOC(=O)[C@H](C)N[P@@](=O)(Oc1ccccc1)[C@@H](F)c1ccc2sc(C(=O)N[C@H]3C[C@@H](C)C[C@H]4CC[C@@H](C(=O)N5CC(c6cnccc6OC)C5)N4C3=O)cc2c1] |
| | | CFTE-28A, SFC peak 2 was |
| | | used in synthesis and biological |
| | | testing |
| 1060 | propyl (((R)- fluoro(2- (((3S,6S,9aS)-3-(3- (5-fluoro-4- hydroxypyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 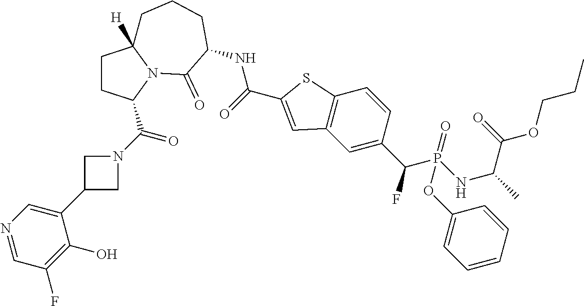 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cncc(F)c6O)C5)N4C3=O)cc2c1] |
| 1061 | propyl (((S)-(2- (((3S,6S,9aS)-3-(3- (4-(3- cyanoazetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)fluoromethyl) (phenoxy) phosphoryl)- L-alaninate | 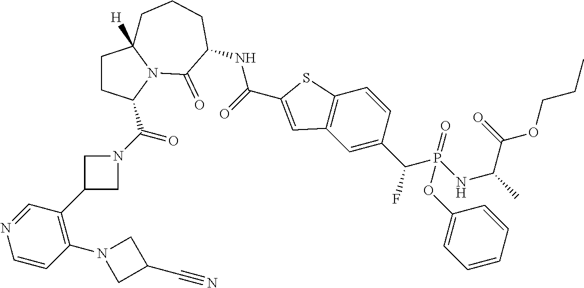 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cnccc6N6CC(C#N)C6)C5)N4C3=O)cc2c1] |
| 1062 | propyl (((R)- fluoro(2- (((3S,6S,9aS)-3-(3- (4-(3- methoxyazetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 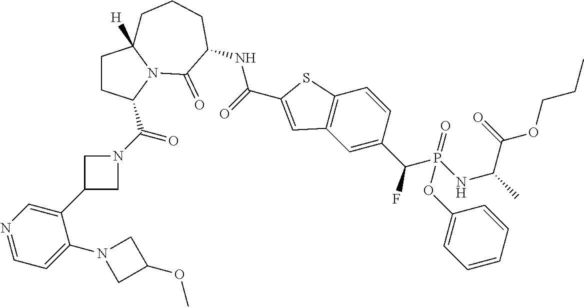 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cnccc6N6CC(OC)C6)C5)N4C3=O)cc2c1] |
| 1063 | propyl (((S)- fluoro(2- (((3S,6S,9aS)-3-(3- (4-(3- methoxyazetidin-1- yl)pyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 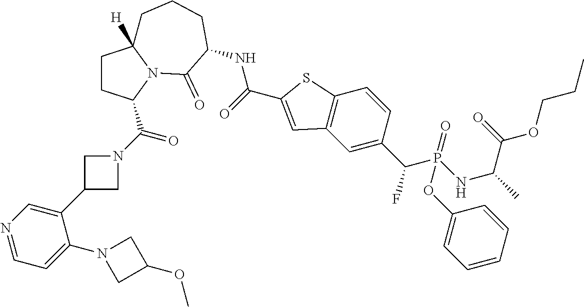 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cnccc6N6CC(OC)C6)C5)N4C3=O)cc2c1] |
| 1064 | propyl (((R)- fluoro(2- (((3S,6S,9aS)-3-(3- (4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 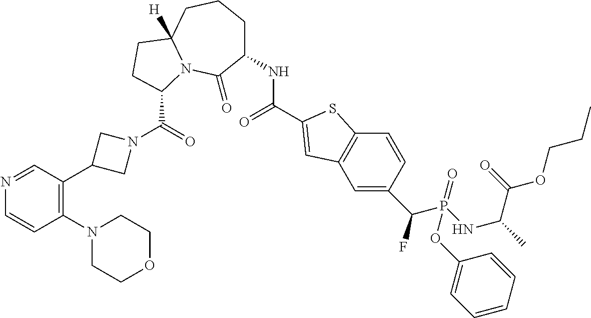 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cnccc6N6CCOCC6)C5)N4C3=O)cc2c1] |
| 1065 | propyl (((S)- fluoro(2- (((3S,6S,9aS)-3-(3-(4- morpholinopyridin- 3-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 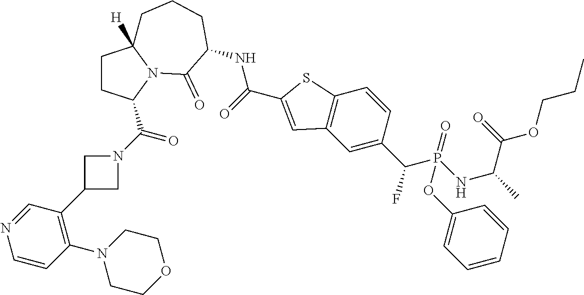 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cnccc6N6CCOCC6)C5)N4C3=O)cc2c1] |
| 1066 | propyl (((R)- fluoro(2- (((3S,6S,9aS)-3-(3- (4- morpholino- pyrimidin- 5-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 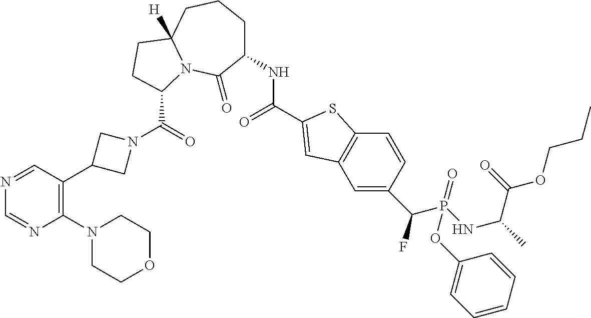 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cncnc6N6CCOCC6)C5)N4C3=O)cc2c1] |
| 1067 | propyl (((S)- fluoro(2- (((3S,6S,9aS)-3-(3- (4- morpholinopyrimidin- 5-yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 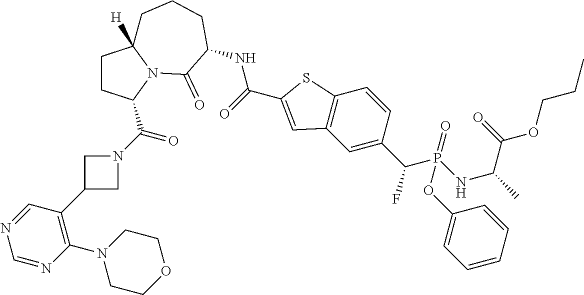 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cncnc6N6CCOCC6)C5)N4C3=O)cc2c1] |
| 1068 | propyl (((S)-(2- (((3S,6S,9aS)-3-(3- (4- (dimethylamino) pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) (phenoxyphosphoryl)- L-alaninate | 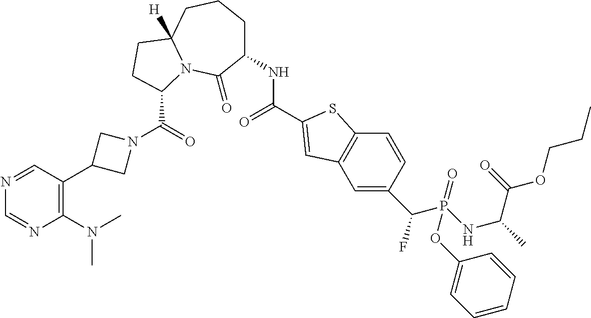 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cncnc6N(C)C)C5)N4C3=O)cc2c1] |
| 1069 | propyl (((R)-(2- (((3S,6S,9aS)-3-(3- (4- (dimethylamino) pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) (phenoxyphosphoryl)- L-alaninate | 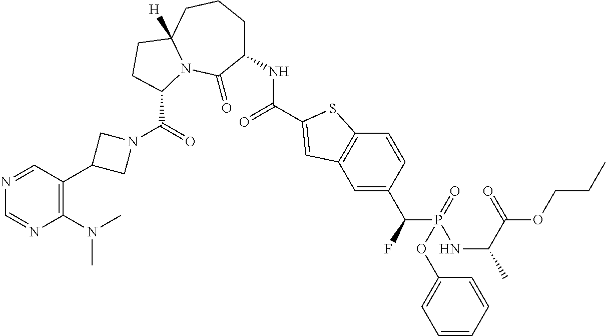 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cncnc6N(C)C)C5)N4C3=O)cc2c1] |
| 1070 | propyl (((2- (((3S,6S,9aS)-3-(3- (4- (dimethylamino) pyrimidin-5- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 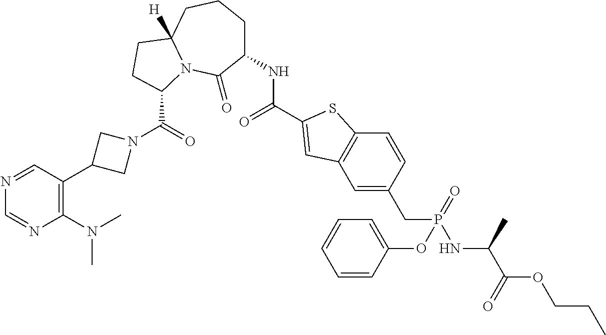 [SMILES: CCCOC(=O)[C@H](C)NP(=O)(Cc1ccc2sc(C(=O)N[C@H]3CCC[C@H]4CC[C@@H](C(=O)N5CC(c6cncnc6N(C)C)C5)N4C3=O)cc2c1)Oc1ccccc1] |
| 1071 | propyl (((R)- fluoro(2- (((3S,6S,9aS)-3-(3- (4-((S)-2- (methoxymethyl) azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 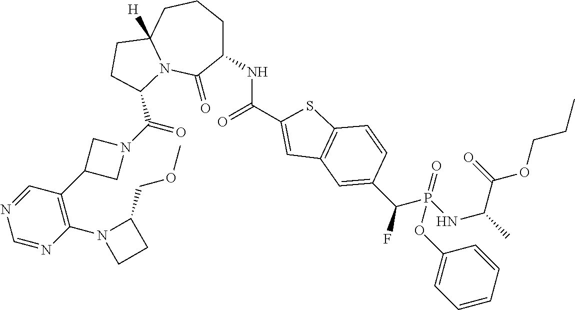 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cncnc6N6CC[C@H]6COC)C5)N4C3=O)cc2c1] |
| 1072 | propyl (((2- (((3S,6S,9aS)-3-(3- (4-((S)-2- (methoxymethyl) azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 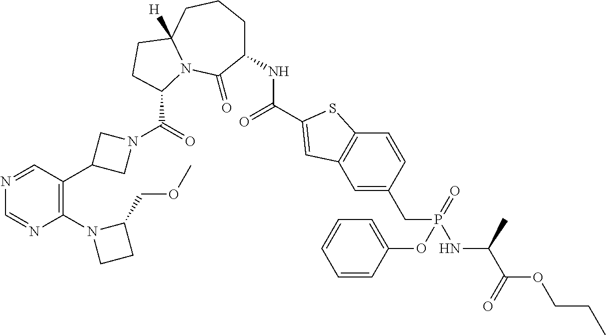 [SMILES: CCCOC(=O)[C@H](C)NP(=O)(Cc1ccc2sc(C(=O)N[C@H]3CCC[C@H]4CC[C@@H](C(=O)N5CC(c6cncnc6N6CC[C@H]6COC)C5)N4C3=O)cc2c1)Oc1ccccc1] |
| 1073 | propyl (((R)- fluoro(2- (((3S,6S,9aS)-3-(3- (5-fluoro-4- methoxypyridin-3- yl)azetidine-1- carbonyl)-5- oxooctahydro-1H- pyrrolo[1,2- a]azepin-6- yl)carbamoyl)benzo [b]thiophen-5- yl)methyl)(phenoxy) phosphoryl)-L- alaninate | 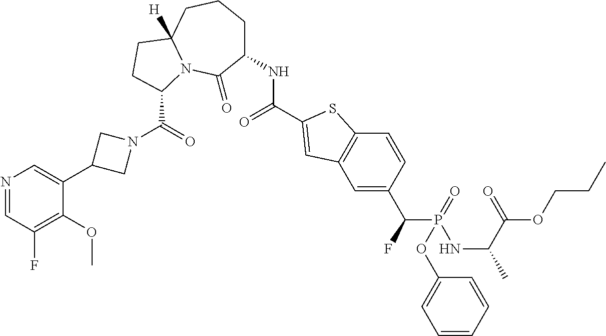 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cncc(F)c6OC)C5)N4C3=O)cc2c1] |
| 1074 | propyl (((2- (((3S,6S,8R,9aR)- 3-(3-(4-(azetidin-1- yl)pyrimidin-5- yl)azetidine-1- carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 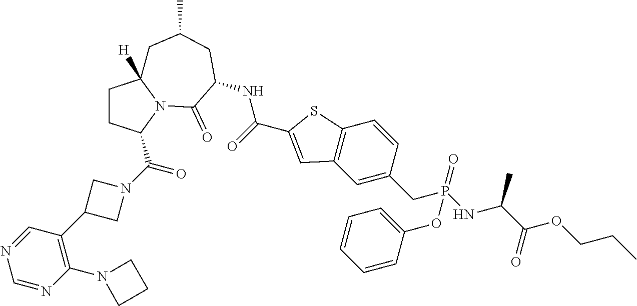 [SMILES: CCCOC(=O)[C@H](C)NP(=O)(Cc1ccc2sc(C(=O)N[C@H]3C[C@@H](C)C[C@H]4CC[C@@H](C(=O)N5CC(c6cncnc6N6CCC6)C5)N4C3=O)cc2c1)Oc1ccccc1] |
| 1075 | propyl (((R)-(2- (((3S,6S,9aS)-3-(3- (4-(azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) (phenoxyphosphoryl)- L-alaninate | 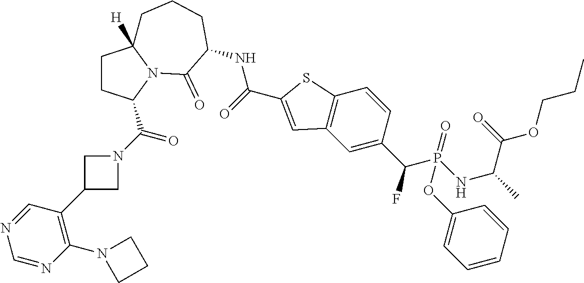 [SMILES: CCCOC(=O)[C@H](C)NP(=O)(Oc1ccccc1)[C@@H](F)c1ccc2sc(C(=O)N[C@H]3CCC[C@H]4CC[C@@H](C(=O)N5CC(c6cncnc6N6CCC6)C5)N4C3=O)cc2c1] |
| 1076 | propyl (((S)-(2- (((3S,6S,9aS)-3-(3- (4-(azetidin-1- yl)pyrimidin-5- yl)azetidine-1- carbonyl)-5- oxooctahydro-1H- pyrrolo[1,2- a]azepin-6- yl)carbamoyl)benzo [b]thiophen-5- yl)fluoromethyl) (phenoxyphosphoryl)- L-alaninate | 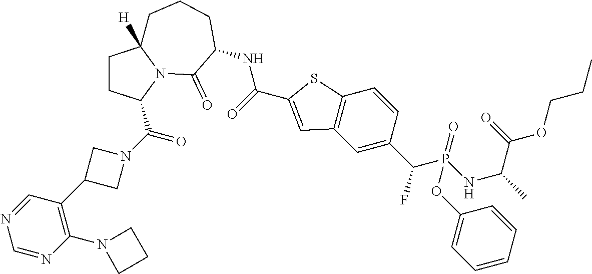 [SMILES: CCCOC(=O)[C@H](C)NP(=O)(Oc1ccccc1)[C@H](F)c1ccc2sc(C(=O)N[C@H]3CCC[C@H]4CC[C@@H](C(=O)N5CC(c6cncnc6N6CCC6)C5)N4C3=O)cc2c1] |
| 1077 | propyl (((R)-(2- (((3S,6S,8R,9aR)- 3-(3-(4-(azetidin-1- yl)pyrimidin-5- yl)azetidine-1- carbonyl)-8- methyl-5- oxooctahydro-1H- pyrrolo[1,2- a]azepin-6- yl)carbamoyl)benzo [b]thiophen-5- yl)fluoromethyl) (phenoxyphosphoryl)- L-alaninate | 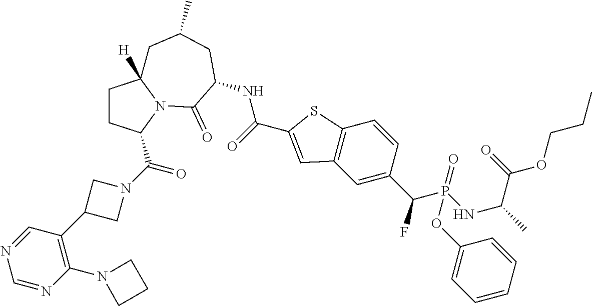 [SMILES: CCCOC(=O)[C@H](C)NP(=O)(Oc1ccccc1)[C@@H](F)c1ccc2sc(C(=O)N[C@H]3C[C@@H](C)C[C@H]4CC[C@@H](C(=O)N5CC(c6cncnc6N6CCC6)C5)N4C3=O)cc2c1] |
| 1078 | propyl (((S)-(2- (((3S,6S,8R,9aR)- 3-(3-(4-(azetidin-1- yl)pyrimidin-5- yl)azetidine-1- carbonyl)-8- methyl-5- oxooctahydro-1H- pyrrolo[1,2- a]azepin-6- yl)carbamoyl)benzo [b]thiophen-5- yl)fluoromethyl) (phenoxyphosphoryl)- L-alaninate | 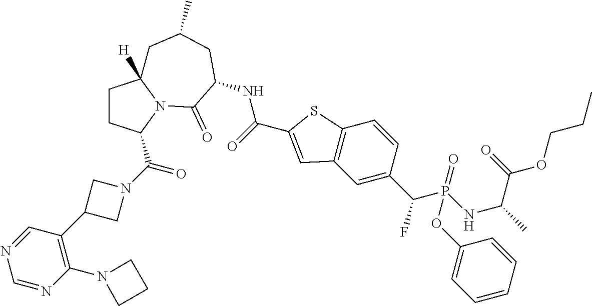 [SMILES: CCCOC(=O)[C@H](C)NP(=O)(Oc1ccccc1)[C@H](F)c1ccc2sc(C(=O)N[C@H]3C[C@@H](C)C[C@H]4CC[C@@H](C(=O)N5CC(c6cncnc6N6CCC6)C5)N4C3=O)cc2c1] |
| 1079 | propyl (((2- (((3S,6S,8S,9aR)-3- (3-(4- (dimethylamino) pyridin-3-yl)azetidine- 1-carbonyl)-8- methyl-5- oxooctahydro-1H- pyrrolo[1,2- a]azepin-6- yl)carbamoyl)benzo [b]thiophen-5- yl)methyl)(phenoxy) phosphoryl)-L- alaninate | 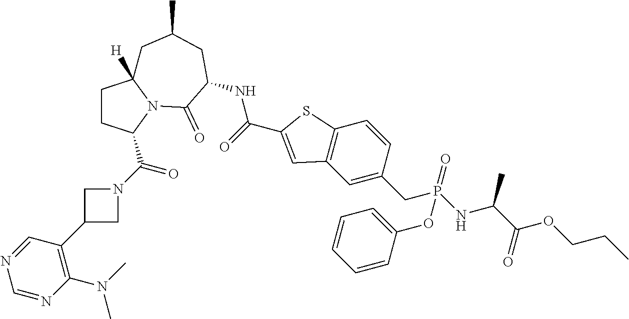 [SMILES: CCCOC(=O)[C@H](C)NP(=O)(Cc1ccc2sc(C(=O)N[C@H]3C[C@H](C)C[C@H]4CC[C@@H](C(=O)N5CC(c6cncnc6N(C)C)C5)N4C3=O)cc2c1)Oc1ccccc1] |
| 1080 | S,S′-(((((2- (((3S,6S,9aS)-3- ((6R,7S)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)difluoromethyl) phosphoryl)bis(oxy)) bis(ethane-2,1- diyl)) bis(2,2- dimethyl- propanethioate) | 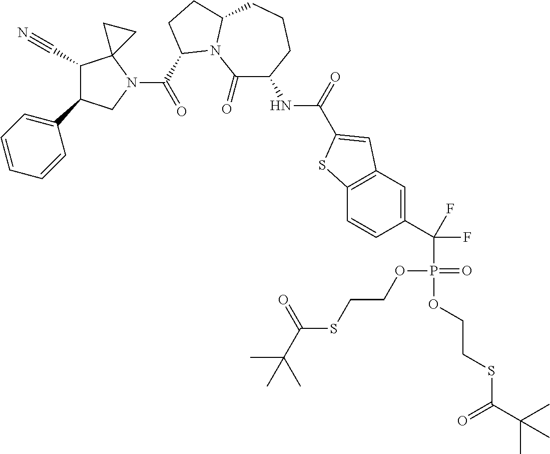 [SMILES: CC(C)(C)C(=O)SCCOP(=O)(OCCSC(=O)C(C)(C)C)C(F)(F)c1ccc2sc(C(=O)N[C@H]3CCC[C@H]4CC[C@@H](C(=O)N5C[C@@H](c6ccccc6)[C@H](C#N)C56CC6)N4C3=O)cc2c1] |
| 1081 | S,S′-(((((2- (((3S,6S,9aS)-3- ((6S,7R)-7-cyano- 6-phenyl-4- azaspiro[2.4]heptane- 4-carbonyl)-5- oxooctahydro-1H- pyrrolo[1,2- a]azepin-6- yl)carbamoyl)benzo [b]thiophen-5- yl)difluoromethyl) phosphoryl)bis(oxy)) bis(ethane-2,1- diyl)) bis(2,2- dimethyl- propanethioate) | 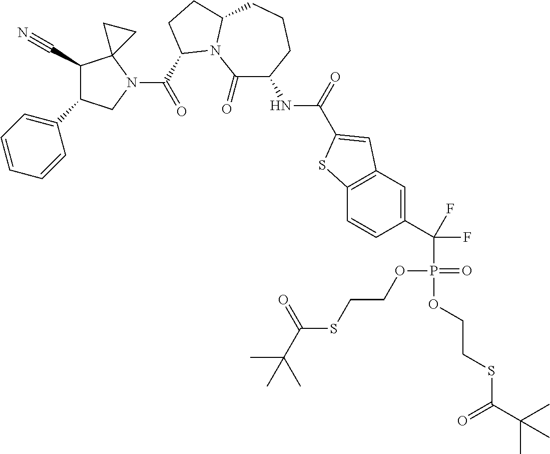 [SMILES: CC(C)(C)C(=O)SCCOP(=O)(OCCSC(=O)C(C)(C)C)C(F)(F)c1ccc2sc(C(=O)N[C@H]3CCC[C@H]4CC[C@@H](C(=O)N5C[C@H](c6ccccc6)[C@@H](C#N)C56CC6)N4C3=O)cc2c1] |
| 1082 | S,S′-((((difluoro(2- (((3S,6S,10aS)-5- oxo-3-(6-phenyl-4- azaspiro[2.4] heptane-4- carbonyl)decahydro pyrrolo[1,2- a]azocin-6- yl)carbamoyl)benzo [b]thiophen-5- yl)methyl) phosphoryl) bis(oxy))bis(ethane- 2,1-diyl)) bis(3- methylbutanethioate) | 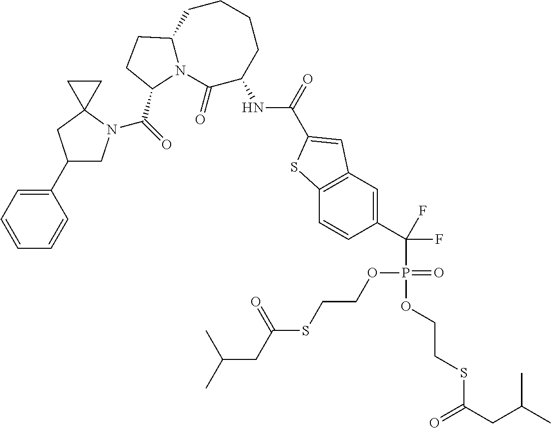 [SMILES: CC(C)CC(=O)SCCOP(=O)(OCCSC(=O)CC(C)C)C(F)(F)c1ccc2sc(C(=O)N[C@H]3CCCC[C@H]4CC[C@@H](C(=O)N5CC(c6ccccc6)CC56CC6)N4C3=O)cc2c1] |
| 1083 | S,S′-(((((2- (((3S,6S,10aS)-3- ((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)difluoromethyl) phosphoryl)bis(oxy)) bis(ethane-2,1- diyl)) bis(3- methoxy- propanethioate) | 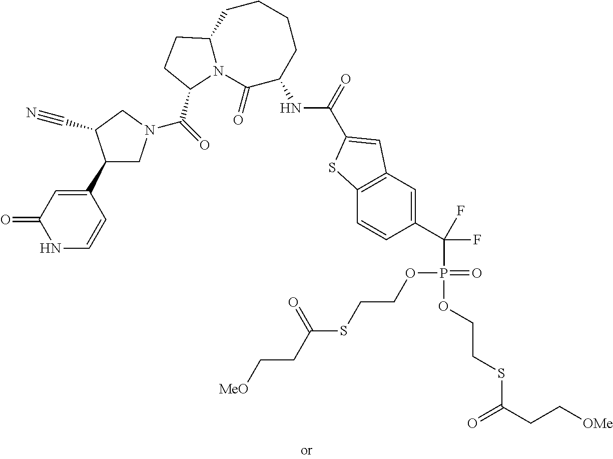 [SMILES: COCCC(=O)SCCOP(=O)(OCCSC(=O)CCOC)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | Or | |
| | S,S′-(((((2- (((3S,6S,10aS)-3- ((3R,4S)-3-cyano- 4-(2-oxo-1,2- dihydropyridin-4- yl)pyrrolidine-1- carbonyl)-5- oxodeca- hydropyrrolo [1,2-a]azocin-6- yl)carbamoyl)benzo [b]thiophen-5- yl)difluoromethyl) phosphoryl)bis(oxy)) bis(ethane-2,1- diyl)) bis(3- methoxy- propanethioate) | 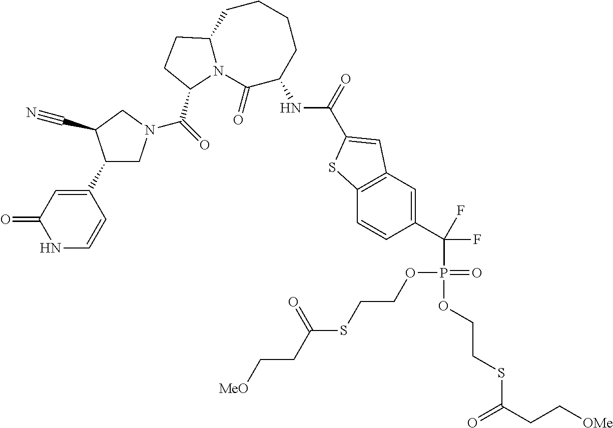 [SMILES: COCCC(=O)SCCOP(=O)(OCCSC(=O)CCOC)C(F)(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | | Pyrrolidine building block SFC |
| | | peak 2 was used for synthesis |
| | | and biological testing |
| 1084 | S,S′-(((((2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) bis(oxy))bis(ethane- 2,1-diyl)) bis(3- methylbutanethioate) | 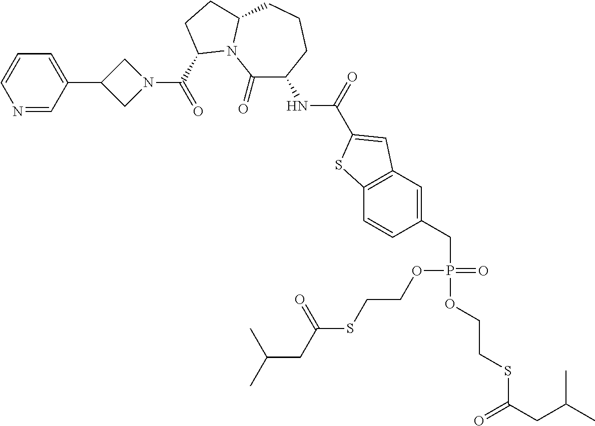 [SMILES: CC(C)CC(=O)SCCOP(=O)(Cc1ccc2sc(C(=O)N[C@H]3CCC[C@H]4CC[C@@H](C(=O)N5CC(c6cccnc6)C5)N4C3=O)cc2c1)OCCSC(=O)CC(C)C] |
| 1085 | S-(2-((((2- (((3S,6S,10aS)-3- ((3R,4S)-3-cyano- 4-(2-oxo-1,2- dihydropyridin-4- yl)pyrrolidine-1- carbonyl)-5- oxodecahydro- pyrrolo [1,2-a]azocin-6- yl)carbamoyl)benzo [b]thiophen-5- yl)difluoromethyl) (((((tetrahydro-2H- pyran-4- yl)oxy)carbonyl) oxy)methoxy) phosphoryl)oxy) ethyl) 3- methylbutanethioate | 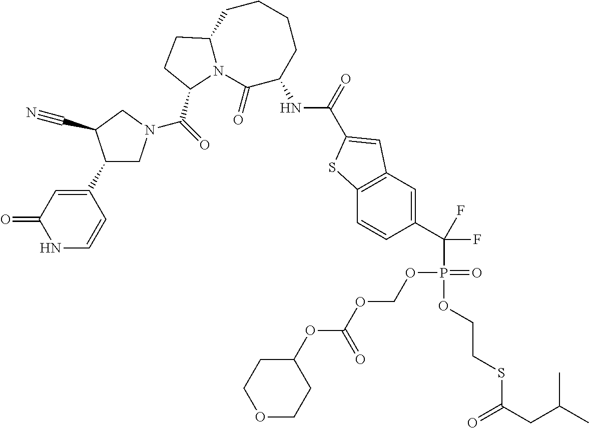 [SMILES: CC(C)CC(=O)SCCOP(=O)(OCOC(=O)OC1CCOCC1)C(F)(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | Or | Or |
| | S-(2-((((2- (((3S,6S,10aS)-3- ((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)difluoromethyl) (((((tetrahydro-2H- pyran-4- yl)oxy)carbonyl) oxy)methoxy) phosphoryl) oxy)ethyl) 3- methylbutanethioate | 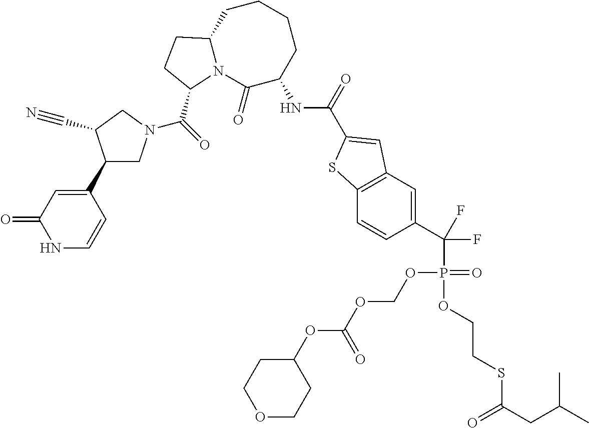 [SMILES: CC(C)CC(=O)SCCOP(=O)(OCOC(=O)OC1CCOCC1)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | | Pyrrolidine building block SFC |
| | | peak 1 was used for synthesis |
| | | and biological testing |
| 1086 | S,S′-((((difluoro(7- (((3S,6S,10aS)-3- ((rel-trans)-3- hydroxy-4-(1H- 1,2,3-triazol-1- yl)pyrrolidine-1- carbonyl)-5- oxodecahydro- pyrrolo [1,2-a]azocin-6- yl)carbamoyl) naphthalen-2- yl)methyl) phosphoryl) bis(oxy))bis(ethane- 2,1-diyl)) bis(3- methylbutanethioate) | 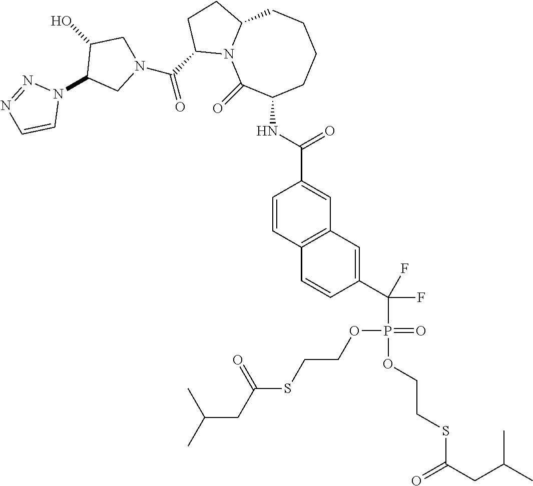 |
| 1087 | ((((7- (((3S,6S,10aS)-3- ((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphoryl)bis(oxy)) bis(methylene) diisopropyl bis(carbonate) Or | 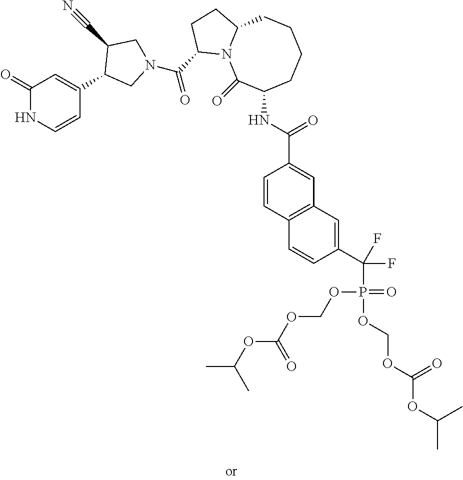 [SMILES: CC(C)OC(=O)OCOP(=O)(OCOC(=O)OC(C)C)C(F)(F)c1ccc2ccc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | ((((7- (((3S,6S,10aS)-3- ((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphoryl)bis(oxy)) bis(methylene) diisopropyl bis(carbonate) | 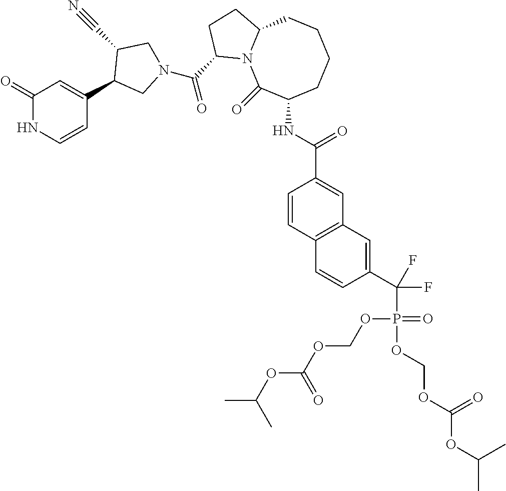 [SMILES: CC(C)OC(=O)OCOP(=O)(OCOC(=O)OC(C)C)C(F)(F)c1ccc2ccc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | | Pyrrolidine building block SFC |
| | | peak 1 was used for synthesis |
| | | and biological testing |
| 1088 | ((((7- (((3S,6S,10aS)-3- ((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphoryl)bis(oxy)) bis(methylene) diisopropyl bis(carbonate) Or | 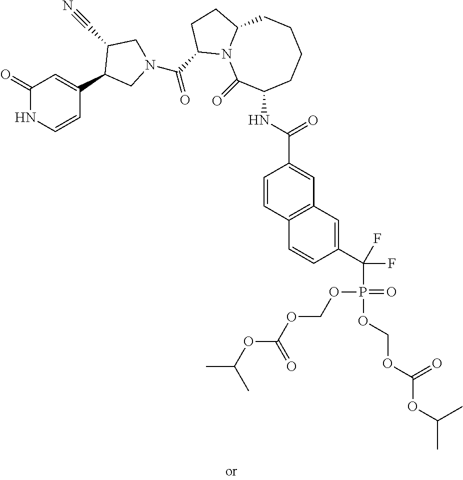 [SMILES: CC(C)OC(=O)OCOP(=O)(OCOC(=O)OC(C)C)C(F)(F)c1ccc2ccc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | ((((7- (((3S,6S,10aS)-3- ((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) phosphoryl)bis(oxy)) bis(methylene) diisopropyl bis(carbonate) | 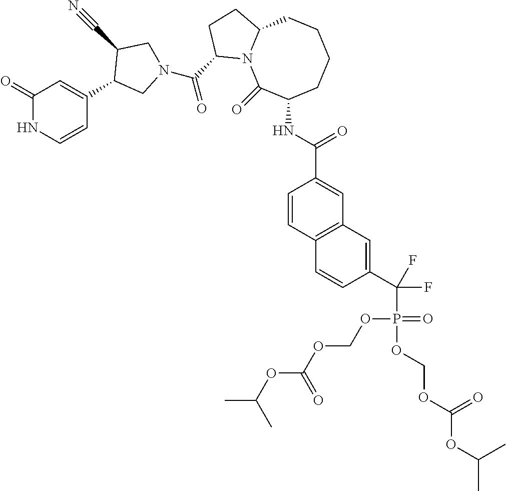 [SMILES: CC(C)OC(=O)OCOP(=O)(OCOC(=O)OC(C)C)C(F)(F)c1ccc2ccc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | | Pyrrolidine building block SFC |
| | | peak 2 was used for synthesis |
| | | and biological testing |
| 1089 | S,S′-(((((R)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) bis(oxy))bis(ethane- 2,1-diyl)) bis(3- methylbutanethioate | 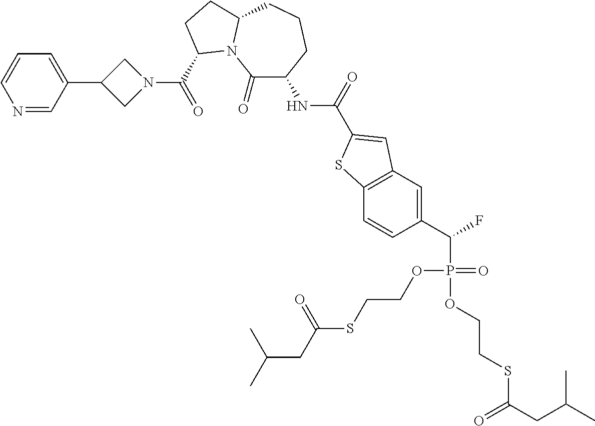 [SMILES: CC(C)CC(=O)SCCOP(=O)(OCCSC(=O)CC(C)C)[C@@H](F)c1ccc2sc(C(=O)N[C@H]3CCC[C@H]4CC[C@@H](C(=O)N5CC(c6cccnc6)C5)N4C3=O)cc2c1] |
| 1090 | S,S′-(((((R)-(2- (((3S,6S,10aS)-3- ((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)fluoromethyl) phosphoryl)bis(oxy)) bis(ethane-2,1-diyl)) bis(3- methylbutanethioate) Or | 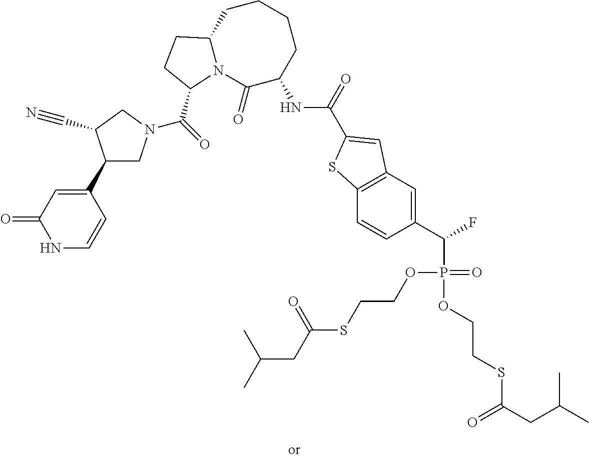 [SMILES: CC(C)CC(=O)SCCOP(=O)(OCCSC(=O)CC(C)C)[C@@H](F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | S,S′-(((((R)-(2- (((3S,6S,10aS)-3- ((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)fluoromethyl) phosphoryl)bis(oxy)) bis(ethane-2,1-diyl)) bis(3- methylbutanethioate) | 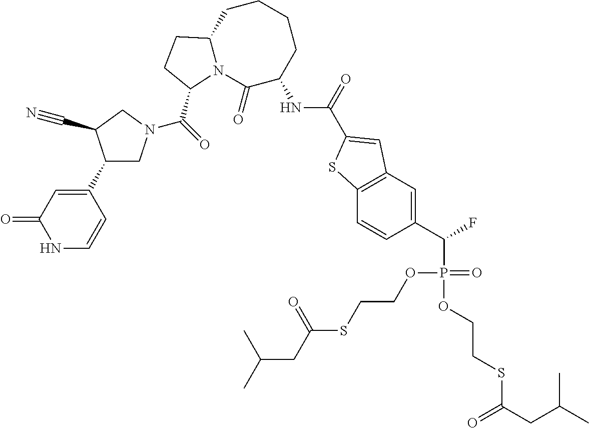 [SMILES: CC(C)CC(=O)SCCOP(=O)(OCCSC(=O)CC(C)C)[C@@H](F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | | Pyrrolidine building block SFC |
| | | peak 2 was used for synthesis |
| | | and biological testing |
| 1091 | S-(2-((((2- (((3S,6S,10aS)-3- ((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)difluoromethyl) (phenoxy)phosphoryl) oxy)ethyl) 3- hydroxy-2,2- dimethyl- propanethioate | 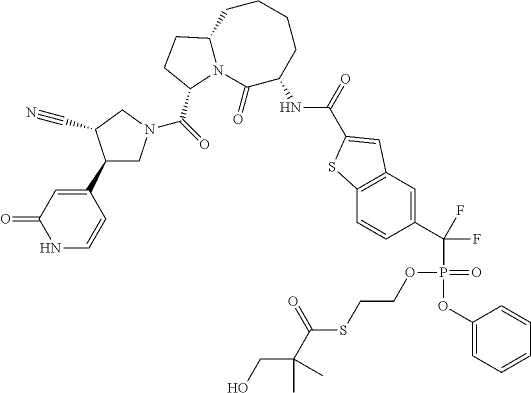 [SMILES: CC(C)(CO)C(=O)SCCOP(=O)(Oc1ccccc1)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | Or | Or |
| | S-(2-((((2- (((3S,6S,10aS)-3- ((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)difluoromethyl) (phenoxy)phosphoryl) oxy)ethyl) 3- hydroxy-2,2- dimethyl- propanethioate | 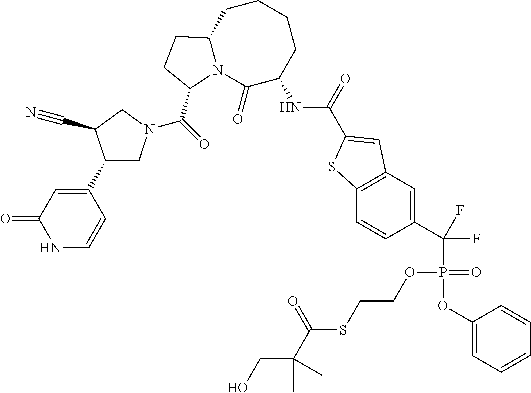 [SMILES: CC(C)(CO)C(=O)SCCOP(=O)(Oc1ccccc1)C(F)(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | | Pyrrolidine building block SFC |
| | | peak 2 was used for synthesis |
| | | and biological testing |
| 1092 | S,S′-((((difluoro(7- (((3S,6S,10aS)-5- oxo-3-(6-(2-oxo- 1,2-dihydropyridin- 4-yl)-4- azaspiro[2.4]heptane-4- carbonyl)decahydro pyrrolo[1,2- a]azocin-6- yl)carbamoyl) naphthalen-2- yl)methyl)phosphoryl) bis(oxy))bis(ethane- 2,1-diyl)) bis(3- methylbutanethioate) | 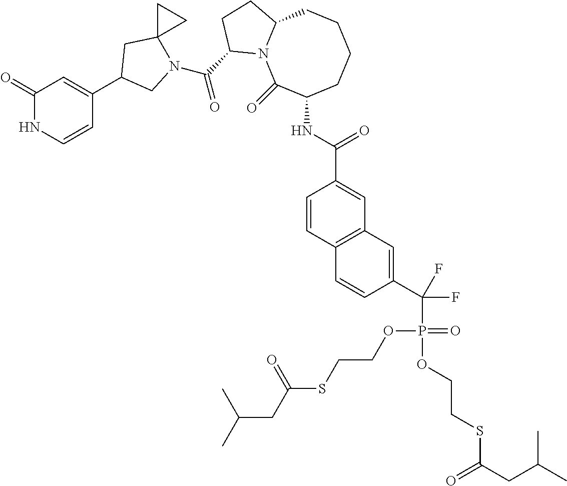 [SMILES: CC(C)CC(=O)SCCOP(=O)(OCCSC(=O)CC(C)C)C(F)(F)c1ccc2ccc(C(=O)N[C@H]3CCCC[C@H]4CC[C@@H](C(=O)N5CC(c6cc[nH]c(=O)c6)CC56CC6)N4C3=O)cc2c1] |
| 1093 | S-(2-((((2- (((3S,6S,10aS)-3- ((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl)benzo [b]thiophen-5- yl)difluoromethyl) ((((oxetan-3- yloxy)carbonyl)oxy) methoxy)phosphoryl) oxy)ethyl) 3- methylbutanethioate Or | 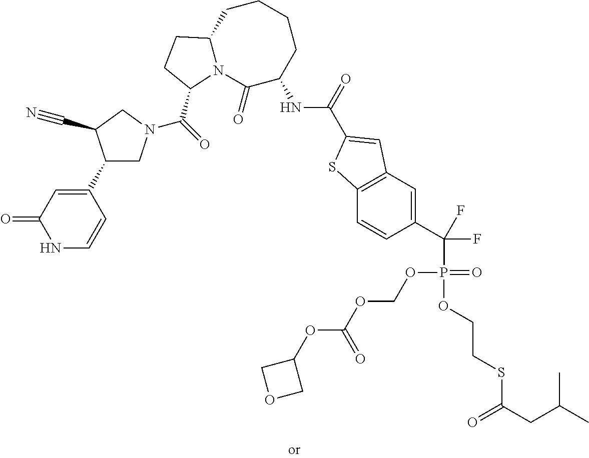 [SMILES: CC(C)CC(=O)SCCOP(=O)(OCOC(=O)OC1COC1)C(F)(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | S-(2-((((2- (((3S,6S,10aS)-3- ((3S,4R)-3-cyano- 4-(2-oxo-1,2- dihydropyridin-4- yl)pyrrolidine-1- carbonyl)-5- oxodecahydro- pyrrolo [1,2-a]azocin-6- yl)carbamoyl)benzo [b]thiophen-5- yl)difluoromethyl) ((((oxetan-3- yloxy)carbonyl)oxy) methoxy)phosphoryl) oxy)ethyl) 3- methylbutanethioate | 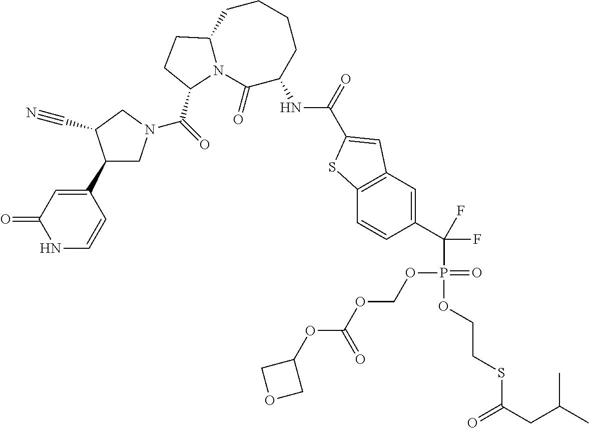 [SMILES: CC(C)CC(=O)SCCOP(=O)(OCOC(=O)OC1COC1)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | | Pyrrolidine building block SFC |
| | | peak 1 was used for synthesis |
| | | and biological testing |
| 1094 | S-(2-((((2- (((3S,6S,10aS)-3- ((3R,4S)-3-cyano- 4-(2-oxo-1,2- dihydropyridin-4- yl)pyrrolidine-1- carbonyl)-5- oxodecahydro- pyrrolo[1,2-a]azocin-6- yl)carbamoyl)benzo [b]thiophen-5- yl)difluoromethyl) ((((((R)- tetrahydrofuran-3- yl)oxy)carbonyl)oxy) methoxy)phosphoryl) oxy)ethyl) 3- methylbutanethioate Or | 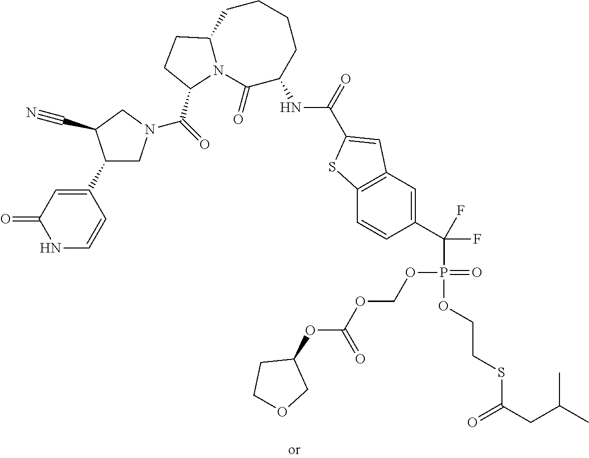 [SMILES: CC(C)CC(=O)SCCOP(=O)(OCOC(=O)O[C@@H]1CCOC1)C(F)(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | S-(2-((((2- (((3S,6S,10aS)-3- ((3S,4R)-3-cyano- 4-(2-oxo-1,2- dihydropyridin-4- yl)pyrrolidine-1- carbonyl)-5- oxodecahydro- pyrrolo [1,2-a]azocin-6- yl)carbamoyl)benzo [b]thiophen-5- yl)difluoromethyl) ((((((R)- tetrahydrofuran-3- yl)oxy)carbonyl) oxy)methoxy) phosphoryl) oxy)ethyl) 3- methyl- butanethioate | 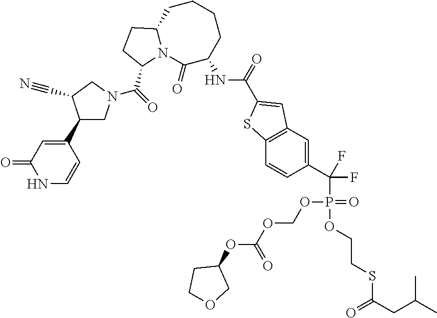 [SMILES: CC(C)CC(=O)SCCOP(=O)(OCOC(=O)O[C@@H]1CCOC1)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | | Pyrrolidine building block SFC |
| | | peak 1 was used for synthesis |
| | | and biological testing |
| 1095 | S-(2-(((difluoro(2- (((3S,6S,9aS)-5- oxo-3-(3- (pyrimidin-2- yl)azetidine-1- carbonyl)octahydro- 1H-pyrrolo[1,2- a]azepin-6- yl)carbamoyl)benzo [b]thiophen-5- yl)methyl) ((((oxetan-3- yloxy)carbonyl) oxy)methoxy) phosphoryl) oxy)ethyl) 3- methyl- butanethioate | 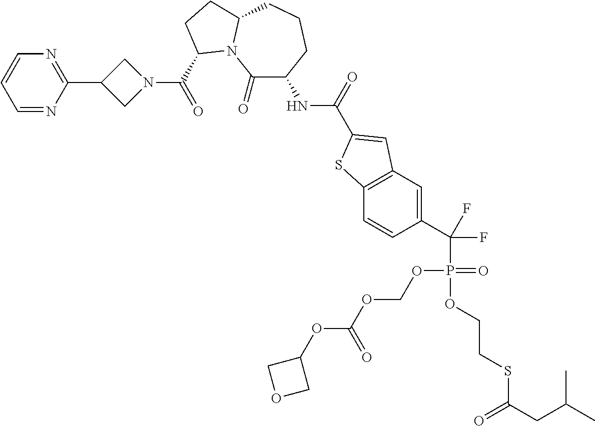 [SMILES: CC(C)CC(=O)SCCOP(=O)(OCOC(=O)OC1COC1)C(F)(F)c1ccc2sc(C(=O)N[C@H]3CCC[C@H]4CC[C@@H](C(=O)N5CC(c6ncccn6)C5)N4C3=O)cc2c1] |
| 1096 | S-(2-((((S)- fluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl) (((isopropoxy- carbonyl)oxy) methoxy)phosphoryl) oxy)ethyl) 3- methylbutanethioate | 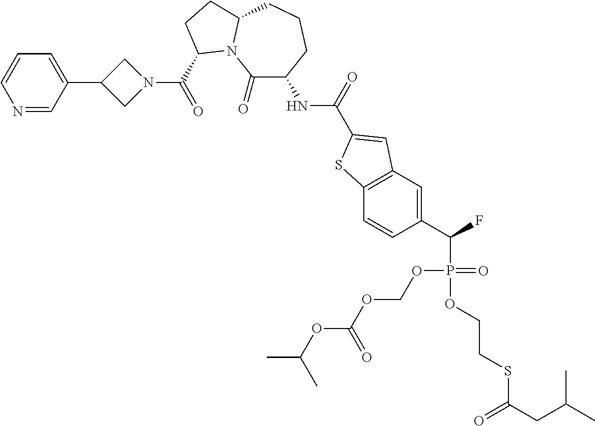 [SMILES: CC(C)CC(=O)SCCOP(=O)(OCOC(=O)OC(C)C)[C@H](F)c1ccc2sc(C(=O)N[C@H]3CCC[C@H]4CC[C@@H](C(=O)N5CC(c6cccnc6)C5)N4C3=O)cc2c1] |
| 1097 | S-(2-((((2- (((3S,6S,10aS)-3- ((3R,4S)-3-cyano- 4-(2-oxo-1,2- dihydropyridin-4- yl)pyrrolidine-1- carbonyl)-5- oxodecahydro- pyrrolo [1,2-a]azocin-6- yl)carbamoyl)benzo [b]thiophen-5- yl)difluoromethyl) ((((((S)- tetrahydrofuran-3- yl)oxy)carbonyl) oxy)methoxy) phosphoryl) oxy)ethyl) 3- methylbutanethioate | 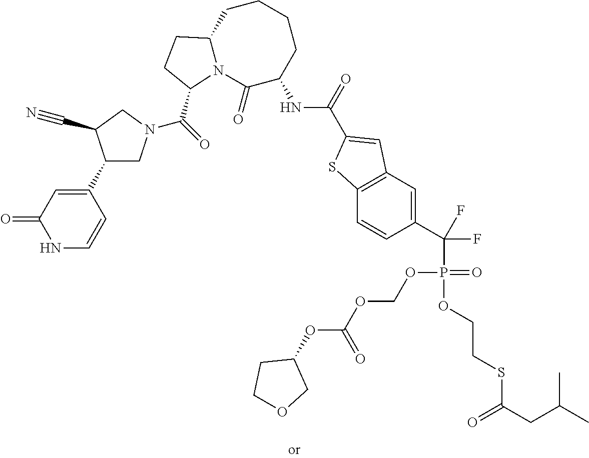 [SMILES: CC(C)CC(=O)SCCOP(=O)(OCOC(=O)O[C@H]1CCOC1)C(F)(F)c1ccc2sc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | S-(2-((((2- (((3S,6S,10aS)-3- ((3S,4R)-3-cyano- 4-(2-oxo-1,2- dihydropyridin-4- yl)pyrrolidine-1- carbonyl)-5- oxodecahydro- pyrrolo [1,2-a]azocin-6- yl)carbamoyl)benzo [b]thiophen-5- yl)difluoromethyl) ((((((S)- tetrahydrofuran-3- yl)oxy)carbonyl) oxy)methoxy) phosphoryl) oxy)ethyl) 3- methylbutanethioate | 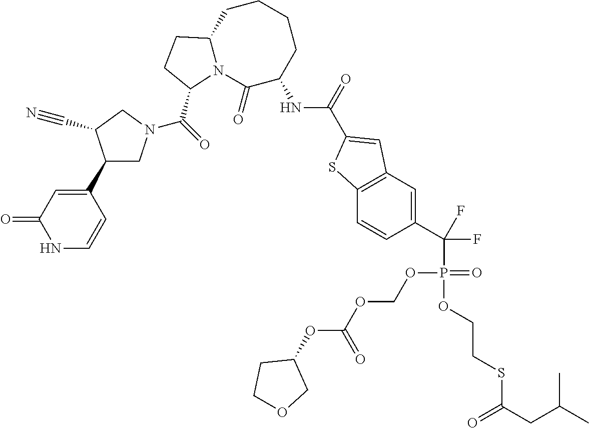 [SMILES: CC(C)CC(=O)SCCOP(=O)(OCOC(=O)O[C@H]1CCOC1)C(F)(F)c1ccc2sc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | | Pyrrolidine building block SFC |
| | | peak 1 was used for synthesis |
| | | and biological testing |
| 1098 | S-(2-(((difluoro(2- (((3S,6S,9aS)-5- oxo-3-(3- (pyrimidin-2- yl)azetidine-1- carbonyl)octahydro- 1H-pyrrolo[1,2- a]azepin-6- yl)carbamoyl)benzo [b]thiophen-5- yl)methyl)((((((R)- tetrahydrofuran-3- yl)oxy)carbonyl) oxy)methoxy) phosphoryl) oxy)ethyl) 3- methylbutanethioate | 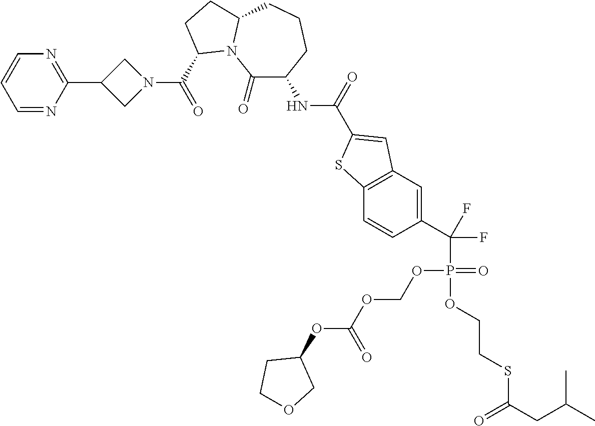 [SMILES: CC(C)CC(=O)SCCOP(=O)(OCOC(=O)O[C@@H]1CCOC1)C(F)(F)c1ccc2sc(C(=O)N[C@H]3CCC[C@H]4CC[C@@H](C(=O)N5CC(c6ncccn6)C5)N4C3=O)cc2c1] |
| 1099 | S-(2-(((difluoro(2- (((3S,6S,9aS)-5- oxo-3-(3- (pyrimidin-2- yl)azetidine-1- carbonyl)octahydro- 1H-pyrrolo[1,2- a]azepin-6- yl)carbamoyl)benzo [b]thiophen-5- yl)methyl)((((((S)- tetrahydrofuran-3- yl)oxy)carbonyl)oxy) methoxy)phosphoryl) oxy)ethyl) 3- methylbutanethioate | 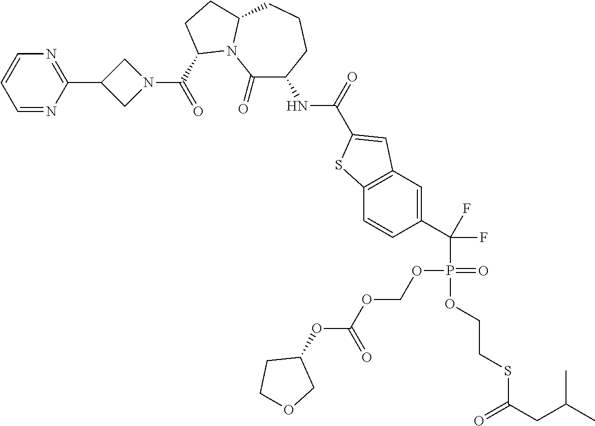 [SMILES: CC(C)CC(=O)SCCOP(=O)(OCOC(=O)O[C@H]1CCOC1)C(F)(F)c1ccc2sc(C(=O)N[C@H]3CCC[C@H]4CC[C@@H](C(=O)N5CC(c6ncccn6)C5)N4C3=O)cc2c1] |
| 1100 | S-(2-((((7- (((3S,6S,10aS)-3- ((3R,4S)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) (((isopropoxy- carbonyl) oxy)methoxy) phosphoryl) oxy)ethyl) 3- methylbutanethioate Or | 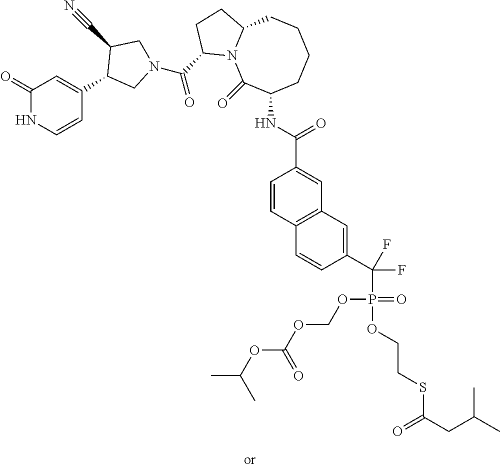 [SMILES: CC(C)CC(=O)SCCOP(=O)(OCOC(=O)OC(C)C)C(F)(F)c1ccc2ccc(C(=O)N[C@H]3CCCC[C@H]4CC[C@@H](C(=O)N5C[C@H](c6cc[nH]c(=O)c6)[C@@H](C#N)C5)N4C3=O)cc2c1] |
| | S-(2-((((7- (((3S,6S,10aS)-3- ((3S,4R)-3-cyano- 4-(2-oxo-1,2- dihydropyridin-4- yl)pyrrolidine-1- carbonyl)-5- oxodecahydropyrrolo [1,2-a]azocin-6- yl)carbamoyl) naphthalen-2- yl)difluoromethyl) (((isopropoxy- carbonyl) oxy)methoxy) phosphoryl) oxy)ethyl) 3- methylbutanethioate | 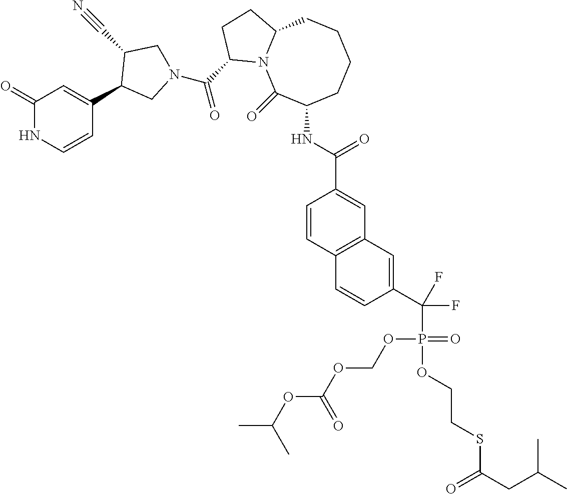 [SMILES: CC(C)CC(=O)SCCOP(=O)(OCOC(=O)OC(C)C)C(F)(F)c1ccc2ccc(C(=O)N[C@H]3CCCC[C@H]4CC[C@@H](C(=O)N5C[C@@H](C#N)[C@H](c6cc[nH]c(=O)c6)C5)N4C3=O)cc2c1] |
| | | Pyrrolidine building block SFC |
| | | peak 1 was used for synthesis |
| | | and biological testing |
| 1101 | S,S′-((((difluoro(7- (((3S,6S,10aS)-5- oxo-3-(6-phenyl-4- azaspiro[2.4] heptane-4- carbonyl)decahydro pyrrolo[1,2- a]azocin-6- yl)carbamoyl) naphthalen-2- yl)methyl)phosphoryl) bis(oxy))bis(ethane- 2,1-diyl)) bis(3- methylbutanethioate) | 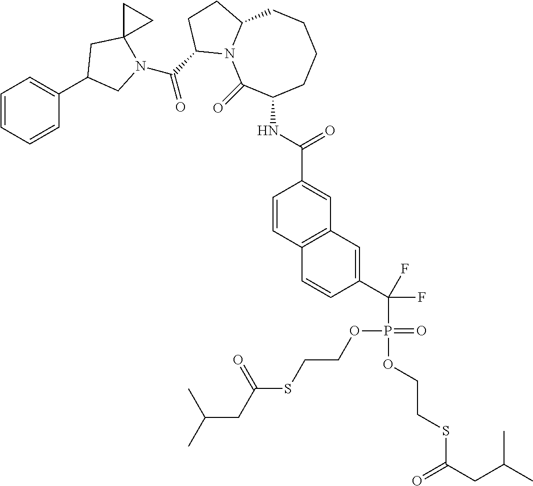 [SMILES: CC(C)CC(=O)SCCOP(=O)(OCCSC(=O)CC(C)C)C(F)(F)c1ccc2ccc(C(=O)N[C@H]3CCCC[C@H]4CC[C@@H](C(=O)N5CC(c6ccccc6)CC56CC6)N4C3=O)cc2c1] |
| 1102 | S,S′-((((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) bis(oxy))bis(ethane- 2,1-diyl)) bis(2-(2- methoxyethoxy) ethanethioate) | 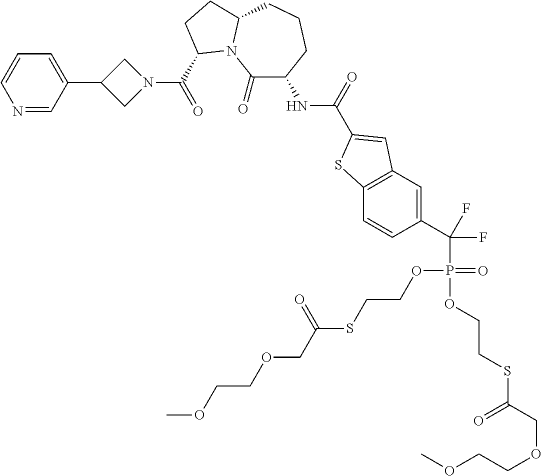 [SMILES: COCCOCC(=O)SCCOP(=O)(OCCSC(=O)COCCOC)C(F)(F)c1ccc2sc(C(=O)N[C@H]3CCC[C@H]4CC[C@@H](C(=O)N5CC(c6cccnc6)C5)N4C3=O)cc2c1] |
| 1103 | S,S′-(((((3-((E)-3- (((3S,6S,10aS)-3- ([1,1′-biphenyl]-4- ylcarbamoyl)-5- oxodecahydropyrrolo [1,2-a]azocin-6- yl)amino)-3- oxoprop-1-en-1- yl)phenyl)difluoro methylphosphoryl) bis(oxy))bis(ethane- 2,1-diyl)) dibutanethioate | 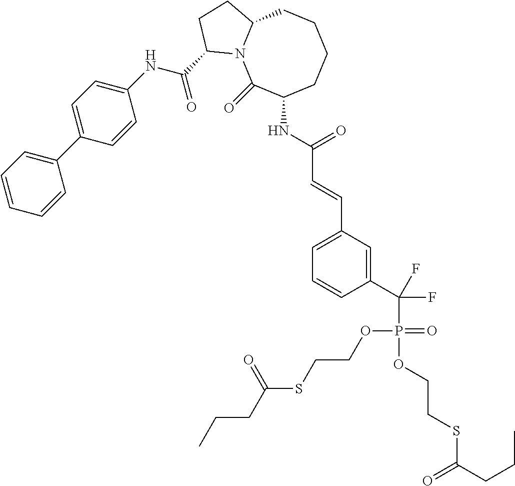 [SMILES: CCCC(=O)SCCOP(=O)(OCCSC(=O)CCC)C(F)(F)c1cccc(/C=C/C(=O)N[C@H]2CCCC[C@H]3CC[C@@H](C(=O)Nc4ccc(-c5ccccc5)cc4)N3C2=O)c1] |
| 1104 | ((((3-((E)-3- (((3S,6S,10aS)-3- ([1,1′-biphenyl]-4- ylcarbamoyl)-5- oxodecahydropyrrolo [1,2-a]azocin-6- yl)amino)-3- oxoprop-1-en-1- yl)phenyl)difluoro methylphosphoryl) bis(oxy))bis (methylene) bis(2,2- dimethylpropanoate) | 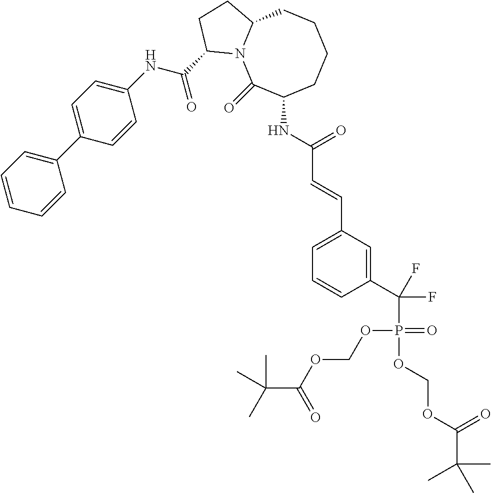 [SMILES: CC(C)(C)C(=O)OCOP(=O)(OCOC(=O)C(C)(C)C)C(F)(F)c1cccc(/C=C/C(=O)N[C@H]2CCCC[C@H]3CC[C@@H](C(=O)Nc4ccc(-c5ccccc5)cc4)N3C2=O)c1] |
| 1105 | S-(2-(((difluoro(2- (((3S,6S,9aS)-5- oxo-3-(3- (pyrimidin-2- yl)azetidine-1- carbonyl)octahydro- 1H-pyrrolo[1,2- a]azepin-6- yl)carbamoyl)benzo [b]thiophen-5- yl)methyl)(((((tetra- hydro-2H-pyran-4- yl)oxy)carbonyl) oxy)methoxy) phosphoryl) oxy)ethyl) 3- methylbutanethioate | 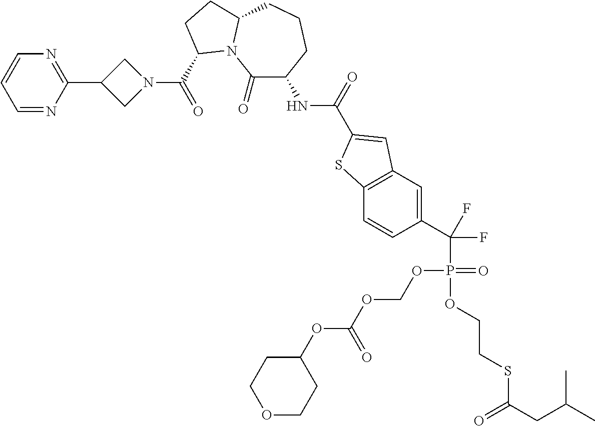 [SMILES: CC(C)CC(=O)SCCOP(=O)(OCOC(=O)OC1CCOCC1)C(F)(F)c1ccc2sc(C(=O)N[C@H]3CCC[C@H]4CC[C@@H](C(=O)N5CC(c6ncccn6)C5)N4C3=O)cc2c1] |
| 1106 | S,S′-((((difluoro(2- (((3S,6S,9aS)-5- oxo-3-(3-(pyridin- 3-yl)azetidine-1- carbonyl)octahydro- 1H-pyrrolo[1,2- a]azepin-6- yl)carbamoyl)benzo [b]thiophen-5- yl)methyl)phosphoryl) bis(oxy))bis(ethane- 2,1-diyl)) bis(3- methoxy- propanethioate) | 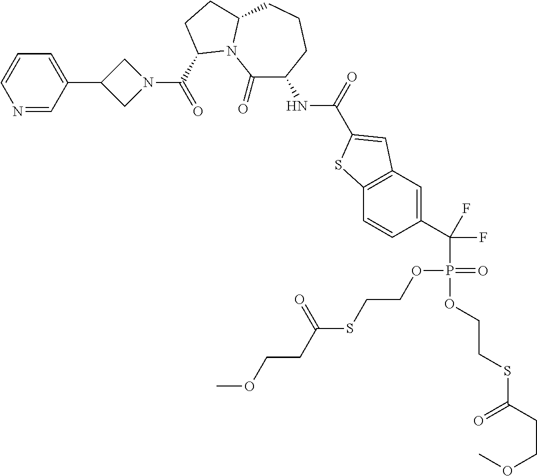 [SMILES: COCCC(=O)SCCOP(=O)(OCCSC(=O)CCOC)C(F)(F)c1ccc2sc(C(=O)N[C@H]3CCC[C@H]4CC[C@@H](C(=O)N5CC(c6cccnc6)C5)N4C3=O)cc2c1] |

| | | LCMS | |
|---|---|---|---|
| | Compound | [M + H]⁺ | NMR |
| | 940 | 880.4 | ¹H NMR (400 MHz, |
| | | | CDCl₃) δ 8.85 − 8.73 |
| | | | (m, 2H), 8.18 − 8.09 |
| | | | (m, 1H), 8.03 − 7.95 |
| | | | (m, 1H), 7.92 − 7.84 |
| | | | (m, 1H), 7.79 − 7.68 |
| | | | (m, 2H), 7.28 − 7.22 |
| | | | (m, 1H), 4.97 − 4.84 |
| | | | (m, 1H), 4.73 − 4.55 |
| | | | (m, 4H), 4.32 − 4.18 |
| | | | (m, 6H), 3.98 − 3.91 |
| | | | (m, 1H), 3.19 − 3.02 |
| | | | (m, 4H), 2.36 − 2.28 |
| | | | (m, 5H), 2.23 (d, J = |
| | | | 8.0 Hz, 1H), 2.06 (d, |
| | | | J = 4.0 Hz, 4H), 2.01 − |
| | | | 1.92 (m, 2H), 1.88 − |
| | | | 1.72 (m, 3H), 1.66 |
| | | | (d, J = 7.2 Hz, 3H), |
| | | | 0.99 − 0.93 (m, 6H) |
| | 941 | 833.5 | ¹H NMR (400 MHz, |
| | | | CDCl₃) δ 8.84 − 8.53 |
| | | | (m, 1H), 8.27 (d, J = |
| | | | 1.2 Hz, 1H), 8.12 − |
| | | | 7.79 (m, 3H), 7.68 − |
| | | | 7.32 (m, 2H), 7.30 − |
| | | | 7.27 (m, 1H), 7.27 − |
| | | | 7.15 (m, 2H), 7.14 − |
| | | | 7.04 (m, 2H), 6.92 − |
| | | | 6.79 (m, 1H), 6.08 − |
| | | | 5.85 (m, 1H), 5.09 − |
| | | | 4.89 (m, 1H), 4.85 − |
| | | | 4.59 (m, 2H), 4.57 − |
| | | | 4.32 (m, 2H), 4.20 − |
| | | | 4.10 (m, 1H), 4.09 − |
| | | | 3.91 (m, 5H), 3.60 (s, |
| | | | 1H), 3.16 − 2.98 (m, |
| | | | 6H), 2.27 (d, J = 6.0 |
| | | | Hz, 1H), 2.19 (d, J = |
| | | | 14.0 Hz, 1H), 2.12 − |
| | | | 1.95 (m, 5H), 1.89 − |
| | | | 1.82 (m, 2H), 1.72 − |
| | | | 1.68 (m, 1H), 1.65 − |
| | | | 1.55 (m, 2H), 1.33 − |
| | | | 1.25 (m, 2H), 1.19 − |
| | | | 1.10 (m, 1H), 0.90 |
| | | | (q, J = 8.0 Hz, 3H) |
| | 942 | 833.3 | ¹H NMR (400 MHz, |
| | | | CDCl₃) δ 8.85 − 8.54 |
| | | | (m, 1H), 8.40 − 8.22 |
| | | | (m, 2H), 8.13 − 7.78 |
| | | | (m, 3H), 7.65 − 7.51 |
| | | | (m, 1H), 7.29 (s, 1H), |
| | | | 7.25 (s, 1H), 7.16 − |
| | | | 7.02 (m, 3H), 6.95 − |
| | | | 6.79 (m, 1H), 6.07 − |
| | | | 5.88 (m, 1H), 5.09 − |
| | | | 4.95 (m, 1H), 4.85 − |
| | | | 4.61 (m, 2H), 4.55 − |
| | | | 4.32 (m, 2H), 4.20 − |
| | | | 4.11 (m, 1H), 4.09 − |
| | | | 3.92 (m, 5H), 3.71 − |
| | | | 3.53 (m, 1H), 3.13 (s, |
| | | | 3H), 3.06 (s, 3H), |
| | | | 2.31 − 2.27 (m, 1H), |
| | | | 2.23 − 2.13 (m, 2H), |
| | | | 2.10 − 2.02 (m, 3H), |
| | | | 2.00 − 1.91 (m, 2H), |
| | | | 1.88 − 1.82 (m, 2H), |
| | | | 1.65 − 1.56 (m, 2H), |
| | | | 1.29 (t, J = 7.6 Hz, |
| | | | 3H), 0.90 (q, J = 7.2 |
| | | | Hz, 3H) |
| | 943 | 808.7 | ¹H NMR (400 MHz, |
| | | | CD₃OD) δ 0.80 − |
| | | | 0.94 (m, 3H) 1.03 − |
| | | | 1.10 (m, 1H) 1.16 − |
| | | | 1.27 (m, 3H) 1.46 − |
| | | | 1.60 (m, 2H) 1.78 − |
| | | | 1.92 (m, 4H) 1.93 − |
| | | | 2.00 (m, 2H) 2.06 − |
| | | | 2.19 (m, 2H) 2.26 − |
| | | | 2.36 (m, 1H) 3.72 − |
| | | | 3.94 (m, 2H) 3.95 − |
| | | | 3.99 (m, 1H) 4.03 − |
| | | | 4.25 (m, 4H) 4.38 − |
| | | | 4.52 (m, 2H) 4.55 − |
| | | | 4.60 (m, 1H) 4.69 − |
| | | | 4.80 (m, 3H) 5.98 − |
| | | | 6.21 (m, 1H) 7.12 − |
| | | | 7.39 (m, 6H) 7.55 − |
| | | | 7.69 (m, 1H) 7.94 − |
| | | | 8.03 (m, 1H) 8.07 − |
| | | | 8.13 (m, 2H) 8.44 − |
| | | | 8.56 (m, 1H) 8.58 − |
| | | | 8.79 (m, 1H) |
| | 944 | 772.4 | ¹H NMR (400 MHz, |
| | | | CDCl₃) δ 8.50-8.52 |
| | | | (m, 1 H) 8.48 (s, 1 H) |
| | | | 7.83 (d, J = 41 Hz, 1 |
| | | | H) 7.62-7.73 (m, 4 |
| | | | H) 7.31-7.35 (m, 1 |
| | | | H) 7.20 (s, 3 H) 7.02- |
| | | | 7.17 (m, 3H) 4.91 (t, |
| | | | J = 8.8 Hz, 1 H) 4.52- |
| | | | 4.60 (m, 4 H) 4.27 (t, |
| | | | J = 4.0 Hz, 1 H) 3.92- |
| | | | 3.97 (m, 1 H) 3.79- |
| | | | 3.85 (m, 5 H) 3.60- |
| | | | 3.81 (m, 1 H) 3.31- |
| | | | 3.39 (m, 2 H) 3.10- |
| | | | 3.23 (m, 1 H) 2.10- |
| | | | 2.17 (m, 3 H) 1.87- |
| | | | 1.94 (m, 2H) 1.82- |
| | | | 1.84 (m, 3 H) 1.16- |
| | | | 1.44 (m, 3 H) 1.04- |
| | | | 1.14 (m, 3 H) 0.64- |
| | | | 0.66 (m, 1 H) 0.53- |
| | | | 0.63 (m, 1 H) |
| | 945 | 816.4 | ¹H NMR (400 MHz, |
| | | | CDCl₃) δ 8.61 (s, 1 |
| | | | H) 8.55-8.56 (m, 1 |
| | | | H) 7.83-7.95 (m, 1 |
| | | | H) 7.79-7.82 (m, 2 |
| | | | H) 7.63-7.77 (m, 2 |
| | | | H) 7.43-7.61 (m, 2 |
| | | | H) 7.28-7.43 (m, 1 |
| | | | H) 7.10-7.17 (m, |
| | | | H) 5.01-5.03 (m, 1 |
| | | | H) 4.64-4.69 (m, 1 |
| | | | H) 4.55-4.63 (m, 1 |
| | | | H) 4.51-4.53 (m, 1 |
| | | | H) 4.49-4.50 (m, 1 |
| | | | H) 4.20-4.22(m, 1 H) |
| | | | 4.01-4.03 (m, 1 H) |
| | | | 3.95-3.97 (m, 3 H) |
| | | | 3.84-3.94 (m, 2H) |
| | | | 3.77-3.79 (m, 1 H) |
| | | | 3.48-3.54 (m, 3 H) |
| | | | 3.20-3.43 (m, 2 H) |
| | | | 3.07-3.20 (m, 1 H) |
| | | | 2.07-2.08 (m, 2 H) |
| | | | 2.05-2.07 (m, 4 H) |
| | | | 1.95-1.97 (m, 2 H) |
| | | | 1.92-1.94 (m, 1 H) |
| | | | 1.77-1.85 (m, 3 H) |
| | | | 0.89-0.91 (m, 3 H) |
| | | | 0.84-0.88 (m, 3 H) |
| | 946 | 850.4 | ¹H NMR (400 MHz, |
| | | | CD₃OD) δ 8.7.4 (s, 1 |
| | | | H) 8.58-8.59 (m, 2 |
| | | | H) 8.04-8.05 (m, 1 |
| | | | H) 7.91-8.03 (m, 3 |
| | | | H) 7.47-7.49 (m, 1 |
| | | | H) 7.29-7.32 (m, 7 |
| | | | H) 7.14-7.28 (m, 3H) |
| | | | 4.89-5.04 (m, 3 H) |
| | | | 4.77-4.86 (m, 2 H) |
| | | | 4.73-4.76 (m, 2 H) |
| | | | 4.53-4.55 (m, 1 H) |
| | | | 4.05-4.09 (m, 5 H) |
| | | | 3.51-3.52 (m, 3 H) |
| | | | 3.30-3.31 (m, 1 H) |
| | | | 3.11-3.12 (m, 4 H) |
| | | | 2.01-2.14 (m, 1 H) |
| | | | 1.83-1.99 (m, 6 H) |
| | 947 | 800.4 | ¹H NMR (400 MHz, |
| | | | CD₃OD) δ 8.55-8.57 |
| | | | (m, 1 H) 8.47-8.54 |
| | | | (m, 1 H) 8.07-8.09 |
| | | | (m, 1 H) 7.89-8.06 |
| | | | (m, 3 H) 7.49-7.51 |
| | | | (m, 2 H) 7.29-7.33 |
| | | | (m, 2 H) 7.15-7.19 |
| | | | (m, 3 H) 4.78-4.87 |
| | | | (m, 2 H) 4.60-4.76 |
| | | | (m, 2 H) 4.07-4.59 |
| | | | (m, 1 H) 4.03-4.07 |
| | | | (m, 3 H) 3.3.73-4.06 |
| | | | (m, 3 H) 3.51-3.72 |
| | | | (m, 2 H) 1.84-2.14 |
| | | | (m, 11 H) 1.46-1.48 |
| | | | (m, 2 H) 0.81-0.87 |
| | | | (m, 7 H) 0.68-0.69 |
| | | | (m, 3 H) |
| | 948 | 814.4 | ¹H NMR (400 MHz, |
| | | | CD₃OD) δ 8.51-8.62 |
| | | | (m, 1 H) 8.47-8.49 |
| | | | (m, 1 H) 8.24 (d, J = |
| | | | 8.0 Hz, 1 H) 8.04- |
| | | | 8.21 (m, 1 H) 7.87- |
| | | | 7.89 (m, 2 H) 7.48- |
| | | | 7.52 (m, 2 H) 7.27- |
| | | | 7.48 (m, 2H) 7.12- |
| | | | 7.21 (m, 3 H) 4.77- |
| | | | 4.83 (m, 3 H) 4.56- |
| | | | 4.58 (m, 2 H) 4.46- |
| | | | 4.49 (m, 1 H) 4.04- |
| | | | 4.08 (m, 3 H) 3.58- |
| | | | 3.69 (m, 2 H) 3.52- |
| | | | 3.57 (m, 3 H) 2.14- |
| | | | 2.32 (m, 1 H) 1.78- |
| | | | 2.02 (m, 9 H) 0.86- |
| | | | 0.87 (m, 3 H) 0.84- |
| | | | 0.85 (m, 3 H) 0.67- |
| | | | 0.80 (m, 6 H) |
| | 949 | 786.4 | ¹H NMR (400 MHz, |
| | | | CD₃OD) δ 8.63 (d, J = |
| | | | 10.4 Hz, 1 H) 8.49- |
| | | | 8.61 (m, 1 H) 8.05- |
| | | | 8.26 (m, 1 H) 8.04- |
| | | | 8.05 (m, 1 H) 7.88- |
| | | | 7.90 (m, 2 H) 7.51- |
| | | | 7.54 (m, 1 H) 7.49- |
| | | | 7.56 (m, 1 H) 7.30- |
| | | | 7.32 (m, 2 H) 7.13- |
| | | | 7.19 (m, 3 H) 4.75- |
| | | | 4.77 (m, 2 H) 4.56- |
| | | | 4.59 (m, 2 H) 4.07- |
| | | | 4.43 (m, 1 H) 4.02- |
| | | | 4.05 (m, 3 H) 3.95- |
| | | | 3.97 (m, 2 H) 3.58- |
| | | | 3.95 (m, 1 H) 3.56- |
| | | | 3.58 (m, 1 H) 3.53- |
| | | | 3.56 (m, 1 H) 2.02- |
| | | | 2.14 (m, 1 H) 1.82- |
| | | | 2.01 (m, 10 H) 1.08- |
| | | | 1.15 (m, 3 H) 0.66- |
| | | | 0.73 (m, 6 H) |
| | 950 | 884.6 | ¹H NMR (400 MHz, |
| | | | CD₃OD) δ 8.89 − |
| | | | 8.78 (m, 1H), 8.72 − |
| | | | 8.61 (m, 1H), 8.34 − |
| | | | 8.22 (m, 1H), 8.16 − |
| | | | 8.03 (m, 2H), 8.02 − |
| | | | 7.90 (m, 1H), 7.79 − |
| | | | 7.58 (m, 2H), 7.51 − |
| | | | 7.27 (m, 3H), 7.22 − |
| | | | 7.16 (m, 2H), 6.21 − |
| | | | 6.05 (m, 1H), 6.03 − |
| | | | 5.80 (m, 1H), 5.02 − |
| | | | 4.98 (m, 1H), 4.68 − |
| | | | 4.61 (m, 1H), 4.58 − |
| | | | 4.17 (m, 5H), 4.00 − |
| | | | 3.87 (m, 2H), 3.87 − |
| | | | 3.75 (m, 1H), 3.67 − |
| | | | 3.55 (m, 2H), 2.35 − |
| | | | 2.19 (m, 2H), 2.15 − |
| | | | 2.05 (m, 1H), 2.05 − |
| | | | 1.87 (m, 6H), 1.81 − |
| | | | 1.72 (m, 1H), 1.73 − |
| | | | 1.61 (m, 2H), 1.59 − |
| | | | 1.47 (m, 2H), 1.33 − |
| | | | 1.17 (m, 2H), 1.07 − |
| | | | 1.05 (m, 1H), 0.92 − |
| | | | 0.80 (m, 3H) |
| | 951 | 786.2 | ¹H NMR (400 MHz, |
| | | | CDCl₃) δ 9.24 − 8.89 |
| | | | (m, 1H), 8.71 − 8.56 |
| | | | (m, 1H), 8.16 − 7.92 |
| | | | (m, 1H), 7.86 − 7.77 |
| | | | (m, 2H), 7.65 − 7.53 |
| | | | (m, 1H), 7.48 − 7.35 |
| | | | (m, 1H), 7.34 − 7.27 |
| | | | (m, 2H), 7.18 − 7.08 |
| | | | (m, 3H), 5.13 − 5.01 |
| | | | (m, 1H), 4.84 − 4.77 |
| | | | (m, 1H), 4.75 − 4.69 |
| | | | (m, 2H), 4.50 − 4.39 |
| | | | (m, 2H), 4.29 − 4.06 |
| | | | (m, 2H), 4.05 − 3.90 |
| | | | (m, 5H), 3.51 − 3.41 |
| | | | (m, 2H), 2.50 (s, 3H), |
| | | | 2.35 − 2.24 (m, 1H), |
| | | | 2.24 − 2.14 (m, 1H), |
| | | | 2.11 − 1.98 (m, 4H), |
| | | | 1.95 − 1.75 (m, 4H), |
| | | | 1.62 − 1.50 (m, 2H), |
| | | | 1.21 − 1.12 (m, 3H), |
| | | | 0.91 − 0.83 (m, 3H) |
| | 952 | 818.0 | ¹H NMR (400 MHz, |
| | | | DMSO-d₆) δ 9.07 − |
| | | | 8.77 (m, 2H), 8.72 − |
| | | | 8.52 (m, 1H), 8.39 − |
| | | | 8.27 (m, 1H), 8.20 − |
| | | | 8.03 (m, 2H), 7.73 − |
| | | | 7.10 (m, 7H), 6.30 − |
| | | | 6.05 (m, 2H), 5.33 − |
| | | | 5.09 (m, 2H), 4.84 − |
| | | | 4.64 (m, 2H), 4.59 − |
| | | | 4.43 (m, 2H), 4.28 − |
| | | | 4.15 (m, 2H), 4.05 − |
| | | | 3.99 (m, 1H), 3.96 − |
| | | | 3.88 (m, 2H), 3.80 − |
| | | | 3.75 (m, 1H), 2.26 − |
| | | | 2.15 (m, 1H), 2.12 − |
| | | | 1.92 (m, 3H), 1.91 − |
| | | | 1.65 (m, 6H), 1.56 − |
| | | | 1.38 (m, 2H), 1.31 − |
| | | | 1.10 (m, 3H), 0.98 − |
| | | | 0.76 (m, 3H) |
| | 953 | 833.7 | ¹H NMR (400 MHz, |
| | | | CD₃OD) δ 8.40 − |
| | | | 8.33 (m, 1H), 8.17 − |
| | | | 8.04 (m, 3H), 8.00 − |
| | | | 7.93 (m, 1H), 7.63 |
| | | | (d, J = 8.4 Hz, 1H), |
| | | | 7.40 − 7.30 (m, 2H), |
| | | | 7.24 − 7.15 (m, 3H), |
| | | | 7.14 − 7.08 (m, 1H), |
| | | | 6.20 − 6.03 (m, 1H), |
| | | | 4.77 − 4.69 (m, 2H), |
| | | | 4.58 − 4.50 (m, 2H), |
| | | | 4.49 − 4.40 (m, 1H), |
| | | | 4.37 − 4.30 (m, 1H), |
| | | | 4.13 − 4.02 (m, 2H), |
| | | | 4.02 − 3.83 (m, 3H), |
| | | | 3.16 (s, 6H), 2.35 − |
| | | | 2.28 (m, 1H), 2.19 − |
| | | | 2.06 (m, 2H), 2.06 − |
| | | | 1.98 (m, 2H), 1.98 − |
| | | | 1.90 (m, 2H), 1.88 (s, |
| | | | 1H), 1.87 − 1.79 (m, |
| | | | 2H), 1.59 − 1.50 (m, |
| | | | 2H), 1.23 (d, J = 7.2 |
| | | | Hz, 3H), 0.92 − 0.83 |
| | | | (m, 3H) |
| | 954 | 833.7 | ¹H NMR (400 MHz, |
| | | | CD₃OD) δ 8.42 − |
| | | | 8.31 (m, 1H), 8.18 − |
| | | | 8.05 (m, 3H), 8.03 − |
| | | | 7.95 (m, 1H), 7.69 − |
| | | | 7.60 (m, 1H), 7.41 − |
| | | | 7.30 (m, 2H), 7.25 − |
| | | | 7.15 (m, 3H), 7.14 − |
| | | | 7.08 (m, 1H), 6.20 − |
| | | | 5.99 (m, 1H), 4.78 |
| | | | 4.68 (m, 2H), 4.59 − |
| | | | 4.50 (m, 2H), 4.49 |
| | | | 4.40 (m, 1H), 4.36 − |
| | | | 4.29 (m, 1H), 4.14 − |
| | | | 4.02 (m, 2H), 3.99 − |
| | | | 3.95 (m, 2H), 3.86 − |
| | | | 3.76 (m, 1H), 3.16 (s, |
| | | | 6H), 2.35 − 2.28 (m, |
| | | | 1H), 2.08 (d, J = 2.4 |
| | | | Hz, 2H), 2.03 − 1.98 |
| | | | (m, 2H), 1.98 − 1.90 |
| | | | (m, 2H), 1.88 (s, 1H), |
| | | | 1.86 − 1.79 (m, 2H), |
| | | | 1.63 − 1.50 (m, 2H), |
| | | | 1.14 − 1.02 (m, 3H), |
| | | | 0.90 − 0.84 (m, 3H) |
| | 955 | 820.2 | ¹H NMR (400 MHz, |
| | | | CDCl₃) δ 8.74 (s, |
| | | | 2H), 8.13 − 8.02 (m, |
| | | | 1H), 7.98 (s, 1H), |
| | | | 7.88 − 7.63 (m, 2H), |
| | | | 7.55 (t, J = 7.6 Hz, |
| | | | 1H), 7.38 − 7.32 (m, |
| | | | 2H), 7.26 − 7.17 (m, |
| | | | 3H), 6.04 − 5.89 (m, |
| | | | 1H), 5.05 − 4.87 (m, |
| | | | 2H), 4.81 − 4.59 (m, |
| | | | 2H), 4.54 (t, J = 5.6 |
| | | | Hz, 1H), 4.43 − 4.34 |
| | | | (m, 1H), 4.15 (s, 3H), |
| | | | 4.11 − 4.02 (m, 1H), |
| | | | 4.01 − 3.91 (m, 1H), |
| | | | 3.83 − 3.73 (m, 2H), |
| | | | 3.72 − 3.64 (m, 1H), |
| | | | 2.34 − 2.25 (m, 1H), |
| | | | 2.24 − 2.09 (m, 2H), |
| | | | 2.07 − 1.98 (m, 3H), |
| | | | 1.96 − 1.68 (m, 4H), |
| | | | 1.26 (dd, J = 3.2, 6.4 |
| | | | Hz, 3H), 1.17 (d, J = |
| | | | 6.4 Hz, 3H), 1.11 |
| | | | (dd, J = 6.4, 9.6 Hz, |
| | | | 3H) |
| | 956 | 820.2 | ¹H NMR (400 MHz, |
| | | | CDCl₃) δ 8.97 − 8.70 |
| | | | (m, 2H), 8.09 − 7.94 |
| | | | (m, 2H), 7.90 − 7.79 |
| | | | (m, 1H), 7.70 − 7.54 |
| | | | (m, 1H), 7.27 − 7.16 |
| | | | (m, 3H), 7.14 − 7.05 |
| | | | (m, 3H), 6.04 − 5.89 |
| | | | (m, 1H), 5.03 − 4.97 |
| | | | (m, 1H), 4.91 (dd, J = |
| | | | 6.0, 8.8 Hz, 1H), 4.77 |
| | | | (dd, J = 7.6, 10.0 Hz, |
| | | | 1H), 4.73 − 4.58 (m, |
| | | | 2H), 4.53 (t, J = 5.6 |
| | | | Hz, 1H), 4.41 − 4.34 |
| | | | (m, 1H), 4.21 − 4.13 |
| | | | (m, 1H), 4.12 − 4.08 |
| | | | (m, 3H), 4.08 − 4.02 |
| | | | (m, 1H), 4.00 − 3.91 |
| | | | (m, 1H), 3.84 − 3.65 |
| | | | (m, 1H), 2.33 − 2.24 |
| | | | (m, 1H), 2.12 (dd, J = |
| | | | 6.0, 13.2 Hz, 2H), |
| | | | 2.07 − 1.97 (m, 3H), |
| | | | 1.95 − 1.67 (m, 4H), |
| | | | 1.27 (d, J = 7.1 Hz, |
| | | | 3H), 1.25 − 1.19 (m, |
| | | | 6H) |
| | 957 | 837.4 | ¹H NMR (400 MHz, |
| | | | CDCl₃) δ 7.97 (d, J = |
| | | | 8.4 Hz, 1H), 7.88 (d, |
| | | | J = 8.4 Hz, 1H), 7.82 |
| | | | (s, 1H), 7.74 (d, J = |
| | | | 4.8 Hz, 1H), 7.61 − |
| | | | 7.53 (m, 1H), 7.38 − |
| | | | 7.28 (m, 1H), 7.24 (s, |
| | | | 1H), 7.22 − 7.16 (m, |
| | | | 1H), 7.10 (s, 1H), |
| | | | 7.09 − 7.06 (m, 1H), |
| | | | 6.98 − 6.93 (m, 1H), |
| | | | 6.82 − 6.78 (m, 1H), |
| | | | 6.13 − 5.83 (m, 1H), |
| | | | 4.93 − 4.81 (m, 1H), |
| | | | 4.69 (d, J = 4.4 Hz, |
| | | | 1H), 4.53 (d, J = 11.2 |
| | | | Hz, 2H), 4.50 − 4.32 |
| | | | (m, 1H), 4.27 − 4.18 |
| | | | (m, 1H), 4.18 − 4.09 |
| | | | (m, 2H), 4.08 − 3.90 |
| | | | (m, 3H), 3.82 (d, J = |
| | | | 4.8 Hz, 3H), 3.77 − |
| | | | 3.60 (m, 1H), 2.34 − |
| | | | 2.13 (m, 2H), 2.11 − |
| | | | 2.02 (m, 3H), 2.01 (s, |
| | | | 3H), 1.85 − 1.68 (m, |
| | | | 2H), 1.67 − 1.48 (m, |
| | | | 2H), 1.33 − 1.09 (m, |
| | | | 3H), 0.97 − 0.80 (m, |
| | | | 3H) |
| | 958 | 817.3 | ¹H NMR (400 MHz, |
| | | | CD₃OD) δ 8.13 − |
| | | | 8.05 (m, 2H), 8.02 − |
| | | | 7.93 (m, 1H), 7.68 − |
| | | | 7.61 (m, 1H), 7.38 − |
| | | | 7.28 (m, 2H), 7.24 − |
| | | | 7.11 (m, 3H), 6.18 − |
| | | | 5.98 (m, 1H), 5.08 − |
| | | | 5.01 (m, 1H), 4.94 (s, |
| | | | 1H), 4.47 − 4.33 (m, |
| | | | 3H), 4.00 − 3.94 (m, |
| | | | 1H), 3.94 − 3.87 (m, |
| | | | 1H), 3.84 − 3.81 (m, |
| | | | 1H), 3.74 − 3.64 (m, |
| | | | 2H), 3.61 − 3.55 (m, |
| | | | 1H), 3.47 (d, J = 10.8 |
| | | | Hz, 1H), 2.25 − 2.15 |
| | | | (m, 2H), 2.07 − 1.91 |
| | | | (m, 6H), 1.82 (d, J = |
| | | | 4.0 Hz, 2H), 1.72 − |
| | | | 1.60 (m, 2H), 1.60 − |
| | | | 1.47 (m, 2H), 1.24 − |
| | | | 1.18 (m, 2H), 1.06 |
| | | | (d, J = 7.2 Hz, 1H), |
| | | | 0.91 − 0.82 (m, 3H) |
| | 959 | 848.2 | ¹H NMR (400 MHz, |
| | | | CDCl₃) δ 8.94 − 8.67 |
| | | | (m, 2H), 8.06 − 7.94 |
| | | | (m, 2H), 7.91 − 7.79 |
| | | | (m, 1H), 7.68 − 7.51 |
| | | | (m, 1H), 7.41 − 7.31 |
| | | | (m, 2H), 7.29 (s, 1H), |
| | | | 7.25 (s, 1H), 7.23 − |
| | | | 7.14 (m, 2H), 6.03 − |
| | | | 5.85 (m, 1H), 5.04 − |
| | | | 4.85 (m, 1H), 4.80 − |
| | | | 4.52 (m, 3H), 4.42 − |
| | | | 4.32 (m, 1H), 4.23 − |
| | | | 4.02 (m, 6H), 3.95 |
| | | | (d, J = 8.0 Hz, 1H), |
| | | | 3.88 − 3.72 (m, 3H), |
| | | | 2.34 − 2.24 (m, 1H), |
| | | | 2.23 − 1.98 (m, 5H), |
| | | | 1.95 − 1.69 (m, 4H), |
| | | | 1.16 (dd, J = 7.2, 10.8 |
| | | | Hz, 3H), 0.90 (s, 9H) |
| | 960 | 848.2 | ¹H NMR (400 MHz, |
| | | | CDCl₃) δ 8.94 − 8.66 |
| | | | (m, 2H), 8.07 − 7.94 |
| | | | (m, 2H), 7.91 − 7.81 |
| | | | (m, 1H), 7.70 − 7.56 |
| | | | (m, 1H), 7.29 (s, 1H), |
| | | | 7.25 (s, 2H), 7.17 − |
| | | | 7.06 (m, 3H), 6.09 − |
| | | | 5.91 (m, 1H), 5.05 − |
| | | | 4.88 (m, 1H), 4.80 − |
| | | | 4.51 (m, 3H), 4.43 − |
| | | | 4.33 (m, 1H), 4.21 − |
| | | | 4.04 (m, 5H), 4.00 − |
| | | | 3.92 (m, 1H), 3.90 − |
| | | | 3.57 (m, 4H), 2.34 − |
| | | | 2.25 (m, 1H), 2.22 − |
| | | | 1.97 (m, 5H), 1.96 − |
| | | | 1.68 (m, 4H), 1.31 |
| | | | (d, J = 6.8 Hz, 3H), |
| | | | 0.95 − 0.87 (m, 9H) |
| | 961 | 908.1 | ¹H NMR (400 MHz, |
| | | | CDCl₃) δ 8.80 − 8.73 |
| | | | (m, 2H), 8.15 − 8.07 |
| | | | (m, 1H), 8.00 − 7.93 |
| | | | (m, 1H), 7.89 − 7.83 |
| | | | (m, 1H), 7.75 (d, J = |
| | | | 5.5 Hz, 1H), 7.71 − |
| | | | 7.66 (m, 1H), 7.26 − |
| | | | 7.22 (m, 1H), 4.93 − |
| | | | 4.85 (m, 1H), 4.71 − |
| | | | 4.64 (m, 1H), 4.63 − |
| | | | 4.56 (m, 2H), 4.55 − |
| | | | 4.39 (m, 1H), 4.31 − |
| | | | 4.14 (m, 6H), 3.97 − |
| | | | 3.88 (m, 1H), 3.18 − |
| | | | 2.97 (m, 4H), 2.36 − |
| | | | 2.21 (m, 2H), 2.19 |
| | | | (d, J = 7.0 Hz, 4H), |
| | | | 2.14 − 2.01 (m, 6H), |
| | | | 2.00 − 1.90 (m, 2H), |
| | | | 1.85 − 1.74 (m, 2H), |
| | | | 0.95 (d, J = 6.6 Hz, |
| | | | 12H) |
| | 962 | 908.3 | ¹H NMR (400 MHz, |
| | | | CDCl₃) δ 8.80 − 8.74 |
| | | | (m, 2H), 8.12 − 8.06 |
| | | | (m, 1H), 7.99 − 7.92 |
| | | | (m, 1H), 7.91 − 7.81 |
| | | | (m, 1H), 7.75 − 7.63 |
| | | | (m, 2H), 7.26 − 7.21 |
| | | | (m, 1H), 4.96 − 4.83 |
| | | | (m, 1H), 4.73 − 4.52 |
| | | | (m, 4H), 4.33 − 4.09 |
| | | | (m, 6H), 4.02 − 3.86 |
| | | | (m, 1H), 3.19 − 3.08 |
| | | | (m, 4H), 2.34 − 2.18 |
| | | | (m, 2H), 2.09 − 2.01 |
| | | | (m, 4H), 2.00 − 1.91 |
| | | | (m, 2H), 1.84 − 1.76 |
| | | | (m, 2H), 1.23 (s, |
| | | | 18H) |
| | 963 | 951.3 | ¹H NMR (400 MHz, |
| | | | CDCl₃) δ 8.55 (d, J = |
| | | | 5.6 Hz, 1H), 8.21 − |
| | | | 8.12 (m, 1H), 8.01 − |
| | | | 7.85 (m, 2H), 7.72 − |
| | | | 7.65 (m, 1H), 7.41 − |
| | | | 7.33 (m, 1H), 7.32 − |
| | | | 7.28 (m, 1H), 5.39 − |
| | | | 5.17 (m, 1H), 4.73 − |
| | | | 4.66 (m, 1H), 4.64 − |
| | | | 4.55 (m, 1H), 4.53 − |
| | | | 4.43 (m, 2H), 4.27 − |
| | | | 4.17 (m, 4H), 4.16 − |
| | | | 4.10 (m, 1H), 3.98 − |
| | | | 3.91 (m, 1H), 3.73 − |
| | | | 3.64 (m, 1H), 3.15 − |
| | | | 3.10 (m, 2H), 3.06 − |
| | | | 3.03 (m, 3H), 2.47 − |
| | | | 2.39 (m, 3H), 2.19 |
| | | | (d, J = 7.2 Hz, 6H), |
| | | | 2.04 − 2.01 (m, 2H), |
| | | | 1.87 − 1.83 (m, 2H), |
| | | | 1.55 − 1.49 (m, 4H), |
| | | | 1.45 (d, J = 7.2 Hz, |
| | | | 3H), 0.95 ( d, J = 6.4 |
| | | | Hz, 12H) |
| | 964 | 883.5 | ¹H NMR (400 MHz, |
| | | | CDCl₃) δ 8.30 − 8.17 |
| | | | (m, 1H), 8.17 − 8.10 |
| | | | (m, 1H), 8.00 − 7.92 |
| | | | (m, 1H), 7.89 − 7.75 |
| | | | (m, 1H), 7.73 − 7.65 |
| | | | (m, 2H), 5.01 − 4.92 |
| | | | (m, 1H), 4.89 − 4.83 |
| | | | (m, 1H), 4.72 − 4.53 |
| | | | (m, 3H), 4.28 − 4.16 |
| | | | (m, 5H), 4.08 − 3.88 |
| | | | (m, 2H), 3.74 − 3.60 |
| | | | (m, 3H), 3.18 − 2.98 |
| | | | (m, 4H), 2.29 (t, J = |
| | | | 7.2 Hz, 4H), 2.21 (d, |
| | | | J = 12.4 Hz, 1H), |
| | | | 2.04 (s, 3H), 1.92 (m, |
| | | | 2H), 1.84 (m, 5H), |
| | | | 1.68 − 1.59 (m, 4H), |
| | | | 0.94 (t, J = 7.2 Hz, |
| | | | 6H) |
| | 965 | 798.3 | ¹H NMR (400 MHz, |
| | | | CDCl₃) δ 9.21 − 8.80 |
| | | | (m, 1H), 8.64 − 8.57 |
| | | | (m, 1H), 8.04 − 7.83 |
| | | | (m, 1H), 7.76 − 7.62 |
| | | | (m, 1H), 7.61 − 7.55 |
| | | | (m, 1H), 7.53 − 7.49 |
| | | | (m, 1H), 7.43 − 7.33 |
| | | | (m, 1H), 7.21 (d, J = |
| | | | 8.4 Hz, 1H), 7.05 − |
| | | | 6.89 (m, 1H), 5.66 − |
| | | | 5.54 (m, 4H), 4.91 (t, |
| | | | J = 6.0 Hz, 2H), 4.81 − |
| | | | 4.57 (m, 3H), 4.55 − |
| | | | 4.41 (m, 1H), 4.27 − |
| | | | 3.83 (m, 4H), 3.42 − |
| | | | 3.33 (m, 2H), 2.31 − |
| | | | 2.14 (m, 2H), 2.11 − |
| | | | 2.01 (m, 4H), 1.99 − |
| | | | 1.91 (m, 2H), 1.87 − |
| | | | 1.78 (m, 2H), 1.31 |
| | | | (d, J = 6.0 Hz, 12H) |
| | 966 | 911.2 | ¹H NMR (400 MHz, |
| | | | CDCl₃) δ 8.29 ( s, |
| | | | 1H), 8.18 − 8.10 (m, |
| | | | 1H), 7.97 ( d, J = 8.4 |
| | | | Hz, 1H), 7.89 − 7.80 |
| | | | (m, 1H), 7.75 − 7.63 |
| | | | (m, 2H), 5.09 − 4.95 |
| | | | (m, 1H), 4.94 − 4.82 |
| | | | (m, 1H), 4.71 − 4.64 |
| | | | (m, 1H), 4.62 − 4.51 |
| | | | (m, 2H), 4.29 − 4.17 |
| | | | (m, 5H), 4.09 − 3.86 |
| | | | (m, 2H), 3.75 − 3.63 |
| | | | (m, 3H), 3.17 − 2.99 |
| | | | (m, 4H), 2.29 − 2.22 |
| | | | (m, 1H), 2.19 (d, J = |
| | | | 7.2 Hz, 4H), 2.13 − |
| | | | 2.08 (m, 2H), 2.06 − |
| | | | 2.01 (m, 5H), 1.96 − |
| | | | 1.89 (m, 2H), 1.87 − |
| | | | 1.80 (m, 1H), 1.76 − |
| | | | 1.66 (m, 1H), 0.95 |
| | | | (d, J = 6.4 Hz, 12H) |
| | 967 | 911.2 | ¹H NMR (400 MHz, |
| | | | CDCl₃) δ 8.34 − 8.09 |
| | | | (m, 2H), 8.01 − 7.93 |
| | | | (m, 1H), 7.87 (s, 1H), |
| | | | 7.80 − 7.67 (m, 2H), |
| | | | 5.10 − 4.77 (m, 2H), |
| | | | 4.72 − 4.52 (m, 3H), |
| | | | 4.28 − 4.12 (m, 5H), |
| | | | 4.05 − 3.88 (m, 2H), |
| | | | 3.74 − 3.62 (m, 3H), |
| | | | 3.20 − 2.97 (m, 4H), |
| | | | 2.31 − 2.15 (m, 2H), |
| | | | 2.11 − 2.05 (m, 2H), |
| | | | 2.00 − 1.89 (m, 4H), |
| | | | 1.87 − 1.62 (m, 2H), |
| | | | 1.19 (s, 18H) |
| | 968 | 921.2 | ¹H NMR (400 MHz, |
| | | | CDCl₃) δ 8.58 − 9.02 |
| | | | (m, 1H) 8.44 (m, 1H) |
| | | | 8.20 (s, 1H) 7.95 (s, |
| | | | 1H) 7.69 (m, 1H) |
| | | | 7.29 − 7.34 (m, 1H) |
| | | | 7.17 (d, J = 4.0 Hz, |
| | | | 1H) 5.00 (s, 1H) 4.61 − |
| | | | 4.80 (m, 2H) 4.53 − |
| | | | 4.60 (m, 1H) 4.29 − |
| | | | 4.47 (m, 1H), 4.12 − |
| | | | 4.24 (m, 6H) 3.94 (s, |
| | | | 1H) 2.99 − 3.15 (m, |
| | | | 4H), 2.30 (s, 3H) |
| | | | 2.12 − 2.21 (m, 2H) |
| | | | 2.00 (s, 5H) 1.82 − |
| | | | 1.91 (m, 3H) 1.17 − |
| | | | 1.19 (m, 18H) |
| | 969 | 848.4 | ¹H NMR (400 MHz, |
| | | | CDCl₃) δ 8.70 − 8.54 |
| | | | (m, 2H), 8.16 (d, J = |
| | | | 9.2 Hz, 1H), 7.67 (s, |
| | | | 5H), 7.46 − 7.34 (m, |
| | | | 1H), 7.24 (t, J = 7.6 |
| | | | Hz, 3H), 7.09 (d, J = |
| | | | 8.4 Hz, 2H), 4.73 − |
| | | | 4.41 (m, 5H), 4.21 |
| | | | (dd, J = 6.4, 8.8 Hz, |
| | | | 1H), 4.13 − 4.05 (m, |
| | | | 1H), 4.02 − 3.87 (m, |
| | | | 4H), 3.52 − 3.28 (m, |
| | | | 2H), 2.31 − 2.11 (m, |
| | | | 3H), 2.10 − 2.00 (m, |
| | | | 5H), 2.00 − 1.91 (m, |
| | | | 3H), 1.83 (d, J = 10.0 |
| | | | Hz, 1H), 1.76 (s, |
| | | | 1H), 1.57 − 1.46 (m, |
| | | | 2H), 1.37 − 1.24 (m, |
| | | | 3H), 0.88 (t, J = 7.6 |
| | | | Hz, 3H) |
| | 970 | 947.1 | ¹H NMR (400 MHz, |
| | | | CDCl₃) δ 9.15 − 8.85 |
| | | | (m, 1H), 8.83 − 8.69 |
| | | | (m, 1H), 8.46 (d, J = |
| | | | 7.2 Hz, 1H), 8.17 (s, |
| | | | 1H), 8.02 (m, 4H), |
| | | | 7.99 − 7.90 (m, 3H), |
| | | | 7.88 − 7.83 (m, 1H), |
| | | | 7.74 − 7.68 (m, 1H), |
| | | | 7.60 − 7.54 (m, 2H), |
| | | | 7.44 (t, J = 7.6 Hz, |
| | | | 4H), 4.88 − 4.67 (m, |
| | | | 3H), 4.55 − 4.41 (m, |
| | | | 6H), 4.20 − 4.14 (m, |
| | | | 1H), 4.04 − 3.92 (m, |
| | | | 2H), 3.28 − 3.20 (m, |
| | | | 4H), 2.34 − 2.26 (m, |
| | | | 1H), 2.23 − 2.12 (m, |
| | | | 2H), 2.09 − 2.03 (m, |
| | | | 3H), 1.98 − 1.81 (m, |
| | | | 4H) |
| | 971 | 872.3 | ¹H NMR (400 MHz, |
| | | | CDCl₃) δ 9.04 − 8.85 |
| | | | (m, 1H), 8.76 − 8.45 |
| | | | (m, 2H), 8.23 − 8.18 |
| | | | (m, 1H), 7.95 − 7.90 |
| | | | (m, 2H), 7.87 − 7.75 |
| | | | (m, 4H), 7.66 − 7.60 |
| | | | (m, 1H), 7.53 − 7.39 |
| | | | (m, 3H), 7.36 − 7.29 |
| | | | (m, 1H), 4.86 − 4.66 |
| | | | (m, 3H), 4.56 − 4.45 |
| | | | (m, 1H), 4.30 − 4.12 |
| | | | (m, 2H), 4.11 − 4.01 |
| | | | (m, 3H), 4.00 − 3.89 |
| | | | (m, 3H), 2.35 − 2.25 |
| | | | (m, 1H), 2.20 − 2.03 |
| | | | (m, 5H), 1.95 − 1.67 |
| | | | (m, 4H), 1.56 − 1.43 |
| | | | (m, 2H), 1.34 − 1.25 |
| | | | (m, 5H), 0.93 − 0.85 |
| | | | (m, 3H) |
| | 972 | 808.4 | ¹H NMR (400 MHz, |
| | | | CDCl₃) δ 8.81 − 8.51 |
| | | | (m, 2H), 8.03 − 7.93 |
| | | | (m, 1H), 7.91 − 7.79 |
| | | | (m, 2H), 7.73 − 7.64 |
| | | | (m, 1H), 7.63 − 7.51 |
| | | | (m, 1H), 7.40 − 7.28 |
| | | | (m, 1H), 7.26 − 7.22 |
| | | | (m, 1H), 7.22 − 7.04 |
| | | | (m, 3H), 6.08 − 5.84 |
| | | | (m, 1H), 5.06 − 4.73 |
| | | | (m, 2H), 4.72 − 4.53 |
| | | | (m, 3H), 4.47 − 4.18 |
| | | | (m, 2H), 4.17 − 4.07 |
| | | | (m, 1H), 3.93 (s, 1H), |
| | | | 3.67 − 3.51 (m, 1H), |
| | | | 2.33 − 2.15 (m, 2H), |
| | | | 2.12 − 2.00 (m, 4H), |
| | | | 1.95 (t, J = 9.2 Hz, |
| | | | 2H), 1.86 − 1.79 (m, |
| | | | 1H), 1.76 − 1.69 (m, |
| | | | 1H), 1.32 − 1.24 (m, |
| | | | 3H), 1.24 − 1.15 (m, |
| | | | 5H), 1.12 − 1.07 (m, |
| | | | 1H) |
| | 973 | 815.3 | ¹H NMR (400 MHz, |
| | | | CD₃OD) δ 8.48 − |
| | | | 8.33 (m, 1H), 8.22 − |
| | | | 8.12 (m, 1H), 8.06 − |
| | | | 7.98 (m, 1H), 7.94 − |
| | | | 7.82 (m, 2H), 7.53 − |
| | | | 7.44 (m, 1H), 7.34 − |
| | | | 7.29 (m, 2H), 7.19 − |
| | | | 7.14 (m, 3H), 7.03 − |
| | | | 6.92 (m, 1H), 4.94 (s, |
| | | | 1H), 4.76 − 4.70 (m, |
| | | | 1H), 4.62 − 4.50 (m, |
| | | | 2H), 4.47 − 4.31 (m, |
| | | | 1H), 4.30 − 4.21 (m, |
| | | | 1H), 4.07 (d, J = 7.6 |
| | | | Hz, 2H), 3.96 − 3.80 |
| | | | (m, 3H), 3.60 − 3.45 |
| | | | (m, 2H), 2.95 − 2.85 |
| | | | (m, 6H), 2.38 − 2.24 |
| | | | (m, 1H), 2.19 − 2.03 |
| | | | (m, 3H), 2.02 − 1.87 |
| | | | (m, 3H), 1.84 (d, J = |
| | | | 12.8 Hz, 3H), 1.59 − |
| | | | 1.48 (m, 2H), 1.19 − |
| | | | 1.05 (m, 3H), 0.94 − |
| | | | 0.82 (m, 3H) |
| | 974 | 806.4 | ¹H NMR (400 MHz, |
| | | | CDCl₃) δ 8.34 − 8.18 |
| | | | (m, 1H), 8.09 − 7.88 |
| | | | (m, 1H), 7.86 − 7.54 |
| | | | (m, 4H), 7.53 − 7.32 |
| | | | (m, 1H), 7.27 (s, 1H), |
| | | | 7.26 − 7.22 (m, 1H), |
| | | | 7.20 − 7.01 (m, 3H), |
| | | | 6.19 − 5.89 (m, 1H), |
| | | | 4.91 − 4.67 (m, 2H), |
| | | | 4.66 − 4.48 (m, 2H), |
| | | | 4.46 − 4.30 (m, 2H), |
| | | | 4.30 − 4.23 (m, 1H), |
| | | | 4.14 − 3.97 (m, 4H), |
| | | | 3.96 − 3.88 (m, 1H), |
| | | | 2.27 (d, J = 8.8 Hz, |
| | | | 1H), 2.15 − 1.96 (m, |
| | | | 5H), 1.95 − 1.83 (m, |
| | | | 2H), 1.66 (s, 2H), |
| | | | 1.65 − 1.51 (m, 2H), |
| | | | 1.38 − 1.19 (m, 3H), |
| | | | 0.99 − 0.82 (m, 3H) |
| | 975 | 788.3 | ¹H NMR (400 MHz, |
| | | | CDCl₃) δ 8.39 − 8.31 |
| | | | (m, 1H), 7.77 − 7.63 |
| | | | (m, 1H), 7.62 − 7.39 |
| | | | (m, 3H), 7.39 − 7.27 |
| | | | (m, 3H), 7.26 − 7.17 |
| | | | (m, 2H), 7.17 − 7.04 |
| | | | (m, 2H), 4.76 − 4.66 |
| | | | (m, 2H), 4.56 (t, J = |
| | | | 7.6 Hz, 1H), 4.42 − |
| | | | 4.32 (m, 2H), 4.32 − |
| | | | 4.24 (m, 1H), 4.19 − |
| | | | 4.00 (m, 3H), 3.99 − |
| | | | 3.87 (m, 3H), 3.65 − |
| | | | 3.37 (m, 2H), 2.33 − |
| | | | 2.21 (m, 1H), 2.19 − |
| | | | 1.95 (m, 6H), 1.92 − |
| | | | 1.76 (m, 2H), 1.74 − |
| | | | 1.61 (m, 2H), 1.61 − |
| | | | 1.50 (m, 1H), 1.29 |
| | | | (d, J = 7.2 Hz, 1H), |
| | | | 1.21 − 1.13 (m, 2H), |
| | | | 0.97 − 0.83 (m, 3H) |
| | 976 | 921.5 | ¹H NMR (400 MHz, |
| | | | CDCl₃) δ 7.98 − 7.91 |
| | | | (m, 1H), 7.89 − 7.83 |
| | | | (m, 1H), 7.77 (s, 1H), |
| | | | 7.64 − 7.51 (m, 1H), |
| | | | 7.44 − 7.32 (m, 2H), |
| | | | 7.23 − 7.17 (m, 3H), |
| | | | 7.15 − 7.02 (m, 2H), |
| | | | 6.78 − 6.63 (m, 1H), |
| | | | 6.06 − 5.83 (m, 1H), |
| | | | 5.52 − 5.42 (m, 1H), |
| | | | 5.17 − 5.07 (m, 1H), |
| | | | 4.40 − 4.30 (m, 1H), |
| | | | 4.16 − 3.93 (m, 3H), |
| | | | 3.57 (s, 3H), 2.65 − |
| | | | 2.53 (m, 1H), 2.52 − |
| | | | 2.39 (m, 2H), 2.30 − |
| | | | 2.16 (m, 2H), 2.04 − |
| | | | 1.83 (m, 6H), 1.63 − |
| | | | 1.57 (m, 4H), 1.35 − |
| | | | 1.24 (m, 5H), 0.96 − |
| | | | 0.81 (m, 4H) |
| | 977 | 903.2 | ¹H NMR (400 MHz, |
| | | | CDCl₃) δ 7.94 (d, J = |
| | | | 12.4 Hz, 1H), 7.90 − |
| | | | 7.83 (m, 1H), 7.80 |
| | | | (d, J = 2.8 Hz, 1H), |
| | | | 7.63 − 7.51 (m, 1H), |
| | | | 7.45 (d, J = 7.2 Hz, |
| | | | 1H), 7.40 − 7.30 (m, |
| | | | 2H), 7.27 − 7.22 (m, |
| | | | 2H), 7.20 − 6.98 (m, |
| | | | 3H), 6.60 (d, J = 4.0 |
| | | | Hz, 1H), 6.13 − 5.83 |
| | | | (m, 1H), 5.47 (t, J = |
| | | | 7.2 Hz, 1H), 5.14 (d, |
| | | | J = 6.0 Hz, 1H), 4.44 − |
| | | | 4.30 (m, 1H), 4.18 − |
| | | | 3.84 (m, 3H), 3.79 − |
| | | | 3.66 (m, 1H), 3.63 (s, |
| | | | 3H), 2.66 − 2.57 (m, |
| | | | 1H), 2.53 − 2.35 (m, |
| | | | 2H), 2.33 − 2.12 (m, |
| | | | 2H), 2.07 − 1.91 (m, |
| | | | 3H), 1.90 − 1.67 (m, |
| | | | 6H), 1.65 −1.58 (m, |
| | | | 2H), 1.57 − 1.48 (m, |
| | | | 2H), 1.36 − 1.11 (m, |
| | | | 3H), 0.97 − 0.81 (m, |
| | | | 3H |
| | 978 | 804.3 | ¹H NMR (400 MHz, |
| | | | DMSO-d₆) δ 8.80 − |
| | | | 8.68 (m, 1H), 8.40 − |
| | | | 8.27 (m, 1H), 8.19 |
| | | | (d, J = 7.8 Hz, 1H), |
| | | | 7.98 − 7.86 (m, 2H), |
| | | | 7.44 (d, J = 8.6 Hz, |
| | | | 1H), 7.35 − 7.29 (m, |
| | | | 2H), 7.20 − 7.10 (m, |
| | | | 4H), 5.75 − 5.55 (m, |
| | | | 1H), 4.77 − 4.54 (m, |
| | | | 2H), 4.48 − 4.43 (m, |
| | | | 1H), 4.46 (d, J = 4.6 |
| | | | Hz, 1H), 4.50 − 4.14 |
| | | | (m, 2H), 4.07 − 3.95 |
| | | | (m, 2H), 3.90 − 3.73 |
| | | | (m, 3H), 3.49 − 3.39 |
| | | | (m, 2H), 2.47 (s, 3H), |
| | | | 2.05 − 1.94 (m, 2H), |
| | | | 1.89 − 1.64 (m, 8H), |
| | | | 1.52 − 1.44 (m, 2H), |
| | | | 1.08 − 0.95 (m, 3H), |
| | | | 0.83 − 0.78 (m, 3H) |
| | 979 | 802.2 | ¹H NMR (400 MHz, |
| | | | CDCl₃) δ 7.90 − 7.81 |
| | | | (m, 1H), 7.81 − 7.70 |
| | | | (m, 4H), 7.66 (s, 1H), |
| | | | 7.46 − 7.35 (m, 1H), |
| | | | 7.31 − 7.28 (m, 1H), |
| | | | 7.18 − 7.08 (m, 4H), |
| | | | 4.75 − 4.57 (m, 3H), |
| | | | 4.52 − 4.41 (m, 2H), |
| | | | 4.40 − 4.29 (m, 1H), |
| | | | 4.11 − 4.02 (m, 2H), |
| | | | 4.01 − 3.94 (m, 4H), |
| | | | 3.89 (d, J = 7.2 Hz, |
| | | | 2H), 3.51 − 3.40 (m, |
| | | | 2H), 2.25 (d, J = 7.2 |
| | | | Hz, 1H), 2.14 − 1.96 |
| | | | (m, 5H), 1.91 (t, J = |
| | | | 9.2 Hz, 2H), 1.79 (s, |
| | | | 2H), 1.62 − 1.51 (m, |
| | | | 2H), 1.47 − 1.37 (m, |
| | | | 1H), 1.22 − 1.14 (m, |
| | | | 3H), 0.91 − 0.83 (m, |
| | | | 3H) |
| | 980 | 819.9 | ¹H NMR (400 MHz, |
| | | | CDCl₃) δ 8.09 (d, J = |
| | | | 5.2 Hz, 1H), 7.90 − |
| | | | 7.76 (m, 3H), 7.74 − |
| | | | 7.65 (m, 1H), 7.45 − |
| | | | 7.41 (m, 1H), 7.31 (s, |
| | | | 1H), 7.22 − 7.10 (m, |
| | | | 3H), 6.86 − 6.76 (m, |
| | | | 1H), 4.87 − 4.76 (m, |
| | | | 1H), 4.74 − 4.66 (m, |
| | | | 1H), 4.62 (d, J = 4.0 |
| | | | Hz, 1H), 4.55 − 4.43 |
| | | | (m, 2H), 4.37 − 4.18 |
| | | | (m, 2H), 4.11 − 3.90 |
| | | | (m, 7H), 3.53 − 3.42 |
| | | | (m, 2H), 2.34 − 2.19 |
| | | | (m, 3H), 1.94 (d, J = |
| | | | 1.2 Hz, 5H), 1.87 − |
| | | | 1.71 (m, 2H), 1.59 |
| | | | (d, J = 7.3, 14.4 Hz, |
| | | | 2H), 1.17 (dd, J = |
| | | | 7.2, 10.9 Hz, 3H), |
| | | | 0.96 − 0.83 (m, 3H) |
| | 981 | 931.3 | ¹H NMR (400 MHz, |
| | | | CDCl₃) δ 7.98 − 7.90 |
| | | | (m, 1H), 7.89 − 7.82 |
| | | | (m, 1H), 7.81 − 7.77 |
| | | | (m, 1H), 7.61 − 7.52 |
| | | | (m, 1H), 7.50 (d, J = |
| | | | 7.6 Hz, 1H), 7.39 − |
| | | | 7.29 (m, 2H), 7.26 − |
| | | | 7.17 (m, 2H), 7.13 − |
| | | | 7.03 (m, 1H), 6.96 (s, |
| | | | 1H), 6.65 − 6.59 (m, |
| | | | 1H), 6.08 − 5.87 (m, |
| | | | 1H), 5.51 − 5.44 (m, |
| | | | 1H), 5.33 − 5.24 (m, |
| | | | 1H), 5.19 − 5.10 (m, |
| | | | 1H), 4.41 − 4.33 (m, |
| | | | 1H), 4.16 − 4.06 (m, |
| | | | 1H), 4.06 − 3.95 (m, |
| | | | 2H), 2.66 − 2.57 (m, |
| | | | 1H), 2.51 − 2.44 (m, |
| | | | 1H), 2.42 − 2.35 (m, |
| | | | 1H), 2.33 − 2.13 (m, |
| | | | 2H), 2.03 − 1.90 (m, |
| | | | 3H), 1.90 − 1.81 (m, |
| | | | 2H), 1.81 − 1.73 (m, |
| | | | 2H), 1.72 − 1.60 (m, |
| | | | 3H), 1.60 − 1.48 (m, |
| | | | 3H), 1.40 (d, J = 6.8 |
| | | | Hz, 6H), 1.32 − 1.25 |
| | | | (m, 2H), 1.12 (d, J = |
| | | | 7.2 Hz, 1H), 1.01 − |
| | | | 0.77 (m, 3H) |
| | 982 | 952.3 | ¹H NMR (400 MHz, |
| | | | CDCl₃) δ 8.72 (s, |
| | | | 1H), 8.59 (d, J = 4.4 |
| | | | Hz, 1H), 7.90 − 7.74 |
| | | | (m, 3H), 7.70 (d, J = |
| | | | 2.6 Hz, 1H), 7.55 − |
| | | | 7.41 (m, 2H), 7.30 − |
| | | | 7.24 (m, 1H), 7.15 − |
| | | | 7.06 (m, 2H), 7.06 − |
| | | | 6.94 (m, 2H), 6.00 − |
| | | | 5.76 (m, 1H), 5.46 − |
| | | | 5.35 (m, 1H), 5.34 − |
| | | | 5.19 (m, 1H), 5.14 − |
| | | | 5.01 (m, 1H), 4.37 − |
| | | | 4.23 (m, 1H), 4.01 − |
| | | | 3.87 (m, 2H), 3.59 − |
| | | | 3.40 (m, 1H), 3.23 − |
| | | | 3.08 (m, 2H), 2.66 − |
| | | | 2.48 (m, 3H), 2.28 − |
| | | | 2.09 (m, 5H), 1.83 − |
| | | | 1.73 (m, 3H), 1.22 − |
| | | | 1.18 (m, 8H), 0.86 − |
| | | | 0.76 (m, 6H) |
| | 983 | 862.4 | ¹H NMR (400 MHz, |
| | | | CDCl34) δ 8.79 |
| | | | 8.52 (m, 2H), 8.03 − |
| | | | 7.94 (m, 1H), 7.92 − |
| | | | 7.79 (m, 2H), 7.68 |
| | | | (d, J = 1.2 Hz, 1H), |
| | | | 7.63 − 7.52 (m, 1H), |
| | | | 7.40 − 7.32 (m, 1H), |
| | | | 7.25 − 7.19 (m, 1H), |
| | | | 7.16 − 7.00 (m, 3H), |
| | | | 6.08 − 5.85 (m, 1H), |
| | | | 5.04 − 4.73 (m, 1H), |
| | | | 4.71 − 4.53 (m, 3H), |
| | | | 4.39 − 4.28 (m, 2H), |
| | | | 4.26 − 4.07 (m, 3H), |
| | | | 4.00 − 3.86 (m, 1H), |
| | | | 3.58 − 3.42 (m, 1H), |
| | | | 2.50 − 2.35 (m, 2H), |
| | | | 2.32 − 2.16 (m, 2H), |
| | | | 2.08 − 2.03 (m, 3H), |
| | | | 2.00 − 1.91 (m, 2H), |
| | | | 1.82 ( dd, J = 3.6, 5.6 |
| | | | Hz, 1H), 1.73 (dt, J = |
| | | | 2.4, 5.2 Hz, 1H), 1.44 |
| | | | ( d, J = 6.8 Hz, 1H), |
| | | | 1.29 ( dd, J = 7.2, |
| | | | 12.4 Hz, 3H) |
| | 984 | 907.3 | ¹H NMR (400 MHz, |
| | | | CDCl₃) δ 8.79 − 8.58 |
| | | | (m, 1H), 8.58 − 8.52 |
| | | | (m, 1H), 7.95 (s, 1H), |
| | | | 7.89 (d, J = 8.4 Hz, |
| | | | 1H), 7.86 (s, 1H), |
| | | | 7.69 (dd, J = 6.0, 11.2 |
| | | | Hz, 1H), 7.54 (d, J = |
| | | | 8.4 Hz, 1H), 7.07 |
| | | | (dd, J = 5.6, 10.0 Hz, |
| | | | 1H), 5.93 − 5.76 (m, |
| | | | 1H), 5.04 − 4.72 (m, |
| | | | 1H), 4.71 − 4.54 (m, |
| | | | 3H), 4.46 − 4.19 (m, |
| | | | 2H), 4.18 − 4.11 (m, |
| | | | 3H), 4.10 − 4.03 (m, |
| | | | 2H), 3.93 (d, J = 3.9 |
| | | | Hz, 1H), 3.16 − 3.01 |
| | | | (m, 4H), 2.32 − 2.15 |
| | | | (m, 2H), 2.11 − 2.06 |
| | | | (m, 1H), 2.00 (s, 2H), |
| | | | 1.99 − 1.86 (m, 3H), |
| | | | 1.82 (d, J = 5.6 Hz, |
| | | | 1H), 1.72 (d, J = 10.4 |
| | | | Hz, 1H), 1.28 − 1.19 |
| | | | (m, 18H) |
| | 985 | 820.2 | ¹H NMR (400 MHz, |
| | | | CD₃OD) δ 8.71 (s, |
| | | | 1H), 8.41 − 8.21 (m, |
| | | | 1H), 8.15 − 8.05 (m, |
| | | | 2H), 8.05 − 7.94 (m, |
| | | | 1H), 7.71 − 7.59 (m, |
| | | | 1H), 7.41 − 7.30 (m, |
| | | | 2H), 7.24 − 7.15 (m, |
| | | | 3H), 7.15 − 7.04 (m, |
| | | | 1H), 6.20 − 6.01 (m, |
| | | | 1H), 4.78 − 4.66 (m, |
| | | | 2H), 4.65 − 4.51 (m, |
| | | | 2H), 4.46 − 4.37 (m, |
| | | | 1H), 4.23 − 4.03 (m, |
| | | | 5H), 4.02 − 3.95 (m, |
| | | | 3H), 3.94 − 3.79 (m, |
| | | | 2H), 2.37 − 2.27 (m, |
| | | | 1H), 2.18 − 2.06 (m, |
| | | | 2H), 2.04 − 1.94 (m, |
| | | | 3H), 1.93 − 1.78 (m, |
| | | | 4H), 1.61 − 1.48 (m, |
| | | | 2H), 1.26 − 1.20 (m, |
| | | | 2H), 1.13 − 1.05 (m, |
| | | | 1H), 0.93 − 0.80 (m, |
| | | | 3H) |
| | 986 | 825.2 | ¹H NMR (400 MHz, |
| | | | CDCl₃) δ 7.98 (d, J = |
| | | | 7.6 Hz, 1H), 7.93 − |
| | | | 7.85 (m, 1H), 7.85 − |
| | | | 7.81 (m, 1H), 7.79 − |
| | | | 7.66 (m, 1H), 7.65 − |
| | | | 7.55 (m, 1H), 7.54 − |
| | | | 7.41 (m, 1H), 7.40 − |
| | | | 7.32 (m, 1H), 7.16 − |
| | | | 6.95 (m, 5H), 6.09 − |
| | | | 5.87 (m, 1H), 4.77 − |
| | | | 4.65 (m, 2H), 4.64 − |
| | | | 4.55 (m, 2H), 4.31 − |
| | | | 3.90 (m, 8H), 3.72 − |
| | | | 3.54 (m, 1H), 2.32 − |
| | | | 2.18 (m, 3H), 1.96 (t, |
| | | | J = 10.2 Hz, 2H), |
| | | | 1.83 (d, J = 8.8 Hz, |
| | | | 1H), 1.78 − 1.70 (m, |
| | | | 1H), 1.70 − 1.60 (m, |
| | | | 2H), 1.59 − 1.49 (m, |
| | | | 1H), 1.33 − 1.25 (m, |
| | | | 3H), 1.14 − 1.11 (m, |
| | | | 1H), 0.97 − 0.89 (m, |
| | | | 3H), 0.89 − 0.82 (m, |
| | | | 1H) |
| | 987 | 877.8 | ¹H NMR (400 MHz, |
| | | | CD₃OD) δ 8.72 − |
| | | | 8.62 (m, 1H), 8.16 − |
| | | | 8.06 (m, 2H), 8.06 − |
| | | | 7.81 (m, 3H), 7.69 − |
| | | | 7.61 (m, 1H), 7.54 − |
| | | | 7.41 (m, 1H), 7.38 − |
| | | | 7.30 (m, 2H), 7.24 − |
| | | | 7.14 (m, 3H), 6.19 − |
| | | | 6.00 (m, 1H), 5.12 − |
| | | | 4.96 (m, 2H), 4.82 − |
| | | | 4.75 (m, 1H), 4.59 − |
| | | | 4.39 (m, 4H), 3.98 (t, |
| | | | J = 3.8, 6.6 Hz, 1H), |
| | | | 3.94 − 3.76 (m, 2H), |
| | | | 3.68 − 3.63 (m, 1H), |
| | | | 3.61 − 3.56 (m, 1H), |
| | | | 3.44 − 3.33 (m, 3H), |
| | | | 3.19 − 3.04 (m, 2H), |
| | | | 2.35 − 2.21 (m, 2H), |
| | | | 2.16 − 1.78 (m, 8H), |
| | | | 1.77 − 1.61 (m, 2H), |
| | | | 1.60 − 1.47 (m, 2H), |
| | | | 1.26 − 1.19 (m, 2H), |
| | | | 1.07 (d, J = 2.6, 7.1 |
| | | | Hz, 1H), 0.92 − 0.82 |
| | | | (m, 3H) |
| | 988 | 818.7 | ¹H NMR (400 MHz, |
| | | | CDCl₃) δ 9.70 − 9.66 |
| | | | (m, 1H), 8.82 − 8.58 |
| | | | (m, 1H), 8.05 − 7.70 |
| | | | (m, 4H), 7.49 − 7.30 |
| | | | (m, 2H), 7.29 (s, 1H), |
| | | | 7.27 − 7.20 (m, 1H), |
| | | | 7.19 − 7.07 (m, 3H), |
| | | | 5.29 − 5.16 (m, 1H), |
| | | | 5.10 − 4.96 (m, 1H), |
| | | | 4.74 − 4.61 (m, 1H), |
| | | | 4.51 − 4.30 (m, 3H), |
| | | | 4.25 − 4.11 (m, 2H), |
| | | | 4.05 − 3.91 (m, 3H), |
| | | | 3.50 − 3.40 (m, 2H), |
| | | | 2.26 − 2.12 (m, 3H), |
| | | | 2.07 − 2.02 (m, 1H), |
| | | | 2.01 − 1.91 (m, 2H), |
| | | | 1.90 − 1.70 (m, 3H), |
| | | | 1.65 − 1.42 (m, 4H), |
| | | | 1.23 − 1.11 (m, 3H), |
| | | | 1.04 (d, J = 6.4 Hz, |
| | | | 3H), 0.93 − 0.82 (m, |
| | | | 3H) |
| | 989 | 875.4 | ¹H NMR (400 MHz, |
| | | | CDCl₃) δ 8.85 − 8.60 |
| | | | (m, 2H), 7.99 − 7.91 |
| | | | (m, 1H), 7.88 − 7.81 |
| | | | (m, 2H), 7.79 − 7.75 |
| | | | (m, 1H), 7.63 − 7.51 |
| | | | (m, 1H), 7.47 (d, J = |
| | | | 3.2 Hz, 1H), 7.38 − |
| | | | 7.31 (m, 1H), 7.26 − |
| | | | 7.15 (m, 3H), 7.14 − |
| | | | 7.00 (m, 2H), 6.06 − |
| | | | 5.85 (m, 1H), 5.56 − |
| | | | 5.45 (m, 1H), 5.20 − |
| | | | 5.07 (m, 1H), 4.42 − |
| | | | 4.32 (m, 1H), 4.21 − |
| | | | 3.76 (m, 3H), 3.65 − |
| | | | 3.47 (m, 1H), 2.60 − |
| | | | 2.55(m, 1H), 2.32 − |
| | | | 2.17 (m, 2H), 2.08 − |
| | | | 1.91 (m, 4H), 1.86 |
| | | | (d, J = 5.4 Hz, 7H), |
| | | | 1.83 − 1.67 (m, 5H), |
| | | | 1.56 − 1.48 (m, 1H), |
| | | | 1.34 − 1.21 (m, 3H), |
| | | | 0.96 − 0.81 (m, 3H) |
| 990 | | 851.9 | ¹H NMR (400 MHz, |
| | | | CDCl₃) δ 8.49 − 8.47 |
| | | | (m, 1H), 7.81 − 7.74 |
| | | | (m, 3H), 7.70 − 7.65 |
| | | | (m, 1H), 7.45 − 7.38 |
| | | | (m, 1H), 7.31 − 7.27 |
| | | | (m, 2H), 7.15 − 7.09 |
| | | | (m, 4H), 6.78-6.65 |
| | | | (m, 1H), 4.85 − 4.77 |
| | | | (m, 1H), 4.66 − 4.58 |
| | | | (m, 2H), 4.52 − 4.51 |
| | | | (m, 2H), 4.46 − 4.41 |
| | | | (m, 1H), 4.25 − 4.18 |
| | | | (m, 3H), 4.00 (s, 1H), |
| | | | 3.97 − 3.92 (m, 3H), |
| | | | 3.50 − 3.42 (m, 4H), |
| | | | 2.29 − 2.20 (m, 2H), |
| | | | 2.07 − 1.95 (m, 6H), |
| | | | 1.84 − 1.70 (m, 2H), |
| | | | 1.56 (d, J = 7.6 Hz, |
| | | | 2H), 1.17 − 1.13 (m, |
| | | | 3H), 0.89 − 0.84 (m, |
| | | | 3H) |
| | 991 | 851.9 | ¹H NMR (400 MHz, |
| | | | CDCl₃) δ 8.53 − 8.43 |
| | | | (m, 1H), 7.81 − 7.76 |
| | | | (m, 3H), 7.73 − 7.66 |
| | | | (m, 1H), 7.46 − 7.39 |
| | | | (m, 1H), 7.30 − 7.28 |
| | | | (m, 1H), 7.26 − 7.25 |
| | | | (m, 1H), 7.19 (d, J = |
| | | | 8.0 Hz, 1H), 7.15 − |
| | | | 7.11 (m, 3H), 4.92 − |
| | | | 4.68 (m, 2H), 4.57 − |
| | | | 4.55 (m, 2H), 4.34 − |
| | | | 4.29 (m, 1H), 4.26 − |
| | | | 4.11 (m, 1H), 4.01 |
| | | | (d, J = 23.2 Hz, 3H), |
| | | | 3.95 − 3.92 (m, 3H), |
| | | | 3.48 − 3.41 (m, 3H), |
| | | | 3.21 − 3.07 (m, 2H), |
| | | | 2.26 − 2.16 (m, 2H), |
| | | | 2.03 − 2.00 (m, 4H), |
| | | | 1.92 − 1.89 (m, 2H), |
| | | | 1.82 − 1.69 (m, 2H), |
| | | | 1.57 − 1.55 (m, 2H), |
| | | | 1.17 − 1.12 (m, 3H), |
| | | | 0.88 − 0.85 (m, 3H) |
| | 992 | 887.4 | ¹H NMR (400 MHz, |
| | | | CDCl₃) δ 8.72 (s, |
| | | | 1H), 8.59 (d, J = 4.4 |
| | | | Hz, 1H), 7.90 − 7.74 |
| | | | (m, 3H), 7.70 (d, J = |
| | | | 2.6 Hz, 1H), 7.55 − |
| | | | 7.41 (m, 2H), 7.30 − |
| | | | 7.24 (m, 1H), 7.15 − |
| | | | 7.06 (m, 2H), 7.06 − |
| | | | 6.94 (m, 2H), 6.00 − |
| | | | 5.76 (m, 1H), 5.46 − |
| | | | 5.35 (m, 1H), 5.34 − |
| | | | 5.19 (m, 1H), 5.14 − |
| | | | 5.01 (m, 1H), 4.37 − |
| | | | 4.23 (m, 1H), 4.01 − |
| | | | 3.87 (m, 2H), 3.59 − |
| | | | 3.40 (m, 1H), 3.23 − |
| | | | 3.08 (m, 2H), 2.66 − |
| | | | 2.48 (m, 3H), 2.28 − |
| | | | 2.09 (m, 5H), 1.83 − |
| | | | 1.73 (m, 3H), 1.22 − |
| | | | 1.18 (m, 8H), 0.86 − |
| | | | 0.76 (m, 6H) |
| | 993 | 818.3 | ¹H NMR (400 MHz, |
| | | | CDCl₃) δ 8.75 − 8.59 |
| | | | (m, 1H), 7.92 − 7.71 |
| | | | (m, 4H), 7.56 − 7.37 |
| | | | (m, 2H), 7.29 (s, 1H), |
| | | | 7.24 − 7.19 (m, 1H), |
| | | | 7.17 − 7.10 (m, 3H), |
| | | | 4.98 − 4.83 (m, 2H), |
| | | | 4.73 − 4.66 (m, 1H), |
| | | | 4.49 − 4.42 (m, 1H), |
| | | | 4.39 − 4.32 (m, 2H), |
| | | | 4.26 − 4.14 (m, 2H), |
| | | | 4.00 − 3.91 (m, 3H), |
| | | | 3.41 (s, 2H), 2.26 − |
| | | | 2.14 (m, 3H), 2.08 − |
| | | | 1.92 (m, 3H), 1.89 − |
| | | | 1.80 (m, 2H), 1.76 − |
| | | | 1.62 (m, 3H), 1.61 − |
| | | | 1.52 (m, 2H), 1.34 − |
| | | | 1.28 (m, 1H), 1.20 − |
| | | | 1.13 (m, 3H), 1.04 |
| | | | (d, J = 6.8 Hz, 3H), |
| | | | 0.92 − 0.83 (m, 3H) |
| | 994 | 808.2 | ¹H NMR (400 MHz, |
| | | | CDCl₃) δ 9.15 − 8.76 |
| | | | (m, 1H), 8.75 − 8.63 |
| | | | (m, 1H), 8.02 − 7.94 |
| | | | (m, 1H), 7.93 − 7.88 |
| | | | (m, 1H), 7.88 − 7.81 |
| | | | (m, 1H), 7.70 − 7.60 |
| | | | (m, 1H), 7.59 − 7.50 |
| | | | (m, 1H), 7.39 − 7.28 |
| | | | (m, 2H), 7.27 − 7.17 |
| | | | (m, 2H), 7.15 − 7.04 |
| | | | (m, 2H), 6.08 − 5.87 |
| | | | (m, 1H), 4.90 (d, J = |
| | | | 6.4 Hz, 1H), 4.79 − |
| | | | 4.76 (m, 1H), 4.70 |
| | | | (d, J = 8.8 Hz, 1H), |
| | | | 4.58 − 4.43 (m, 2H), |
| | | | 4.29 − 4.11 (m, 2H), |
| | | | 4.10 − 4.02 (m, 2H), |
| | | | 4.01 − 3.92 (m, 2H), |
| | | | 3.84 − 3.58 (m, 1H), |
| | | | 2.34 − 2.24 (m, 1H), |
| | | | 2.23 − 2.08 (m, 2H), |
| | | | 2.07 − 2.00 (m, 3H), |
| | | | 1.97 − 1.80 (m, 3H), |
| | | | 1.79 − 1.68 (m, 1H), |
| | | | 1.66 − 1.57 (m, 2H), |
| | | | 1.30 (dd, J = 3.6, 7.2 |
| | | | Hz, 2H), 1.14 (d, J = |
| | | | 7.2 Hz, 1H), 0.94 − |
| | | | 0.87 (m, 3H) |
| | 995 | 878.3 | ¹H NMR (400 MHz, |
| | | | CD₃OD) δ 9.01 − |
| | | | 8.92 (m, 1H), 8.79 − |
| | | | 8.68 (m, 1H), 8.66 − |
| | | | 8.48 (m, 1H), 8.16 − |
| | | | 8.03 (m, 2H), 8.02 − |
| | | | 7.79 (m, 2H), 7.70 − |
| | | | 7.57 (m, 1H), 7.41 − |
| | | | 7.27 (m, 2H), 7.25 − |
| | | | 7.12 (m, 3H), 6.19 − |
| | | | 5.95 (m, 1H), 5.07 − |
| | | | 4.95 (m, 1H), 4.73 − |
| | | | 4.64 (m, 1H), 4.62 − |
| | | | 4.40 (m, 3H), 4.39 − |
| | | | 4.17 (m, 2H), 4.03 − |
| | | | 3.78 (m, 3H), 3.63 − |
| | | | 3.46 (m, 4H), 3.41 − |
| | | | 3.32 (m, 3H), 2.36 − |
| | | | 2.18 (m, 2H), 2.13 − |
| | | | 1.92 (m, 6H), 1.90 − |
| | | | 1.75 (m, 2H), 1.83 − |
| | | | 1.62 (m, 2H), 1.60 − |
| | | | 1.47 (m, 2H), 1.25 − |
| | | | 1.15 (m, 2H), 1.10 − |
| | | | 1.02 (m, 1H), 0.92 − |
| | | | 0.76 (m, 3H) |
| | 996 | 837.9 | ¹H NMR (400 MHz, |
| | | | CD₃OD) 8.68 − 8.52 |
| | | | (m, 1H), 8.51 − 8.41 |
| | | | (m, 1H), 8.03 (d, J = |
| | | | 12.0 Hz, 1H), 7.96 − |
| | | | 7.80 (m, 2H), 7.55 − |
| | | | 7.44 (m, 1H), 7.40 − |
| | | | 7.28 (m, 2H), 7.27 − |
| | | | 7.17 (m, 3H), 7.16 (s, |
| | | | 1H), 7.14 (s, 1H), |
| | | | 4.77 − 4.72 (m, 1H), |
| | | | 4.71 − 4.66 (m, 1H), |
| | | | 4.62 − 4.54 (m, 1H), |
| | | | 4.51 − 4.30 (m, 2H), |
| | | | 4.27 − 4.13 (m, 2H), |
| | | | 4.06 (d, J = 3.8 Hz, |
| | | | 1H), 3.98 − 3.75 (m, |
| | | | 3H), 3.59 − 3.44 (m, |
| | | | 2H), 2.38 − 2.24 (m, |
| | | | 1H), 2.17 − 2.04 (m, |
| | | | 2H), 2.03 − 1.93 (m, |
| | | | 3H), 1.93 − 1.77 (m, |
| | | | 4H), 1.60 − 1.46 (m, |
| | | | 2H), 1.17 − 1.01 (m, |
| | | | 3H), 0.96 − 0.78 (m, |
| | | | 3H |
| | 997 | 808.3 | ¹H NMR (400 MHz, |
| | | | CDCl₃) δ 9.12 − 8.53 |
| | | | (m, 2H), 7.97 (s, 1H), |
| | | | 7.95 − 7.77 (m, 3H), |
| | | | 7.58 (t, J = 8.0 Hz, |
| | | | 1H), 7.26 − 7.19 (m, |
| | | | 2H), 7.14 − 7.06 (m, |
| | | | 3H), 6.04 − 5.88 (m, |
| | | | 1H), 5.10 − 4.83 (m, |
| | | | 1H), 4.79 − 4.61 (m, |
| | | | 2H), 4.60 − 4.51 (m, |
| | | | 1H), 4.49 − 4.22 (m, |
| | | | 2H), 4.22 − 4.01 (m, |
| | | | 4H), 4.00 − 3.91 (m, |
| | | | 1H), 3.74 − 3.59 (m, |
| | | | 1H), 2.11 − 1.99 (m, |
| | | | 5H), 1.98 − 1.72 (m, |
| | | | 5H), 1.68 − 1.58 (m, |
| | | | 2H), 1.31 (d, J = 6.8 |
| | | | Hz, 3H), 0.94 − 0.89 |
| | | | (m, 3H) |
| | 998 | 808.3 | ¹H NMR (400 MHz, |
| | | | CDCl₃) δ 9.17 − 8.61 |
| | | | (m, 2H), 7.97 (s, 1H), |
| | | | 7.92 − 7.81 (m, 2H), |
| | | | 7.58 (t, J = 8.8 Hz, |
| | | | 1H), 7.33 − 7.29 (m, |
| | | | 1H), 7.27 − 7.22 (m, |
| | | | 2H), 7.12 (d, J = 7.6 |
| | | | Hz, 1H), 7.08 (d, J = |
| | | | 7.2 Hz, 2H), 6.08 − |
| | | | 5.84 (m, 1H), 5.10 − |
| | | | 4.86 (m, 1H), 4.80 − |
| | | | 4.60 (m, 2H), 4.59 − |
| | | | 4.50 (m, 1H), 4.49 − |
| | | | 4.35 (m, 1H), 4.30 − |
| | | | 4.11 (m, 3H), 4.07 − |
| | | | 4.03 (m, 2H), 3.97 |
| | | | (d, J = 6.4 Hz, 1H), |
| | | | 3.79 − 3.63 (m, 1H), |
| | | | 2.34 − 2.08 (m, 3H), |
| | | | 2.08 − 1.99 (m, 3H), |
| | | | 1.98 − 1.70 (m, 4H), |
| | | | 1.68 − 1.56 (m, 2H), |
| | | | 1.30 (d, J = 7.2 Hz, |
| | | | 3H), 0.94 − 0.89 (m, |
| | | | 3H) |
| | 999 | 820.2 | ¹H NMR (400 MHz, |
| | | | CDCl₃) 8.95 − 8.66 |
| | | | (m, 2H), 8.08 − 7.92 |
| | | | (m, 2H), 7.92 − 7.62 |
| | | | (m, 2H), 7.61 − 7.54 |
| | | | (m, 1H), 7.26 − 7.18 |
| | | | (m, 2H), 7.17 − 7.06 |
| | | | (m, 3H), 6.05 − 5.88 |
| | | | (m, 1H), 4.94 − 4.84 |
| | | | (m, 1H), 4.80 − 4.67 |
| | | | (m, 1H), 4.66 − 4.57 |
| | | | (m, 1H), 4.56 − 4.49 |
| | | | (m, 1H), 4.42 − 4.33 |
| | | | (m, 1H), 4.21 − 4.11 |
| | | | (m, 2H), 4.09 (d, J = |
| | | | 3.6 Hz, 3H), 4.06 − |
| | | | 4.02 (m, 2H), 4.01 − |
| | | | 3.91 (m, 1H), 3.91 − |
| | | | 3.68 (m, 1H), 2.34 − |
| | | | 2.23 (m, 1H), 2.22 − |
| | | | 2.08 (m, 2H), 2.03 − |
| | | | 1.99 (m, 3H), 1.95 − |
| | | | 1.69 (m, 4H), 1.68 − |
| | | | 1.56 (m, 2H), 1.28 |
| | | | (d, J = 7.2 Hz, 3H), |
| | | | 0.97 − 0.87 (m, 3H) |
| | 1000 | 804.3 | 1H (400 MHz, |
| | | | CDCl₃) δ 9.34 − 8.86 |
| | | | (m, 1H), 8.74 − 8.55 |
| | | | (m, 1H), 8.28 − 8.02 |
| | | | (m, 1H), 7.96 (s, 1H), |
| | | | 7.91 − 7.79 (m, 1H), |
| | | | 7.62 − 7.50 (m, 2H), |
| | | | 7.27 − 7.20 (m, 2H), |
| | | | 7.17 − 7.04 (m, 3H), |
| | | | 6.07 − 5.84 (m, 1H), |
| | | | 5.13 − 5.04 (m, 1H), |
| | | | 4.83 (d, J = 9.2 Hz, |
| | | | 1H), 4.76 − 4.70 (m, |
| | | | 1H), 4.56 − 4.42 (m, |
| | | | 2H), 4.24 − 4.07 (m, |
| | | | 2H), 4.05 − 3.93 (m, |
| | | | 3H), 3.87 − 3.58 (m, |
| | | | 1H), 2.49 (s, 3H), |
| | | | 2.36 − 2.15 (m, 2H), |
| | | | 2.14 − 1.96 (m, 4H), |
| | | | 1.85 (d, J = 3.6 Hz, |
| | | | 3H), 1.65 − 1.59 (m, |
| | | | 2H), 1.46 − 1.39 (m, |
| | | | 1H), 1.33 − 1.25 (m, |
| | | | 3H), 0.97 − 0.86 (m, |
| | | | 3H) |
| | 1001 | 818.2 | ¹H NMR (400 MHz, |
| | | | CDCl₃) δ 9.31 − 8.88 |
| | | | (m, 1H), 8.76 − 8.56 |
| | | | (m, 1H), 8.24 (d, J = |
| | | | 7.6 Hz, 1H), 8.03 (s, |
| | | | 1H), 7.96 (s, 1H), |
| | | | 7.89 − 7.81 (m, 1H), |
| | | | 7.66 − 7.54 (m, 2H), |
| | | | 7.27 − 7.21 (m, 2H), |
| | | | 7.15 − 7.04 (m, 3H), |
| | | | 6.03 − 5.90 (m, 1H), |
| | | | 5.15 − 5.05 (m, 1H), |
| | | | 4.88 − 4.67 (m, 2H), |
| | | | 4.54 (t, J = 4.4 Hz, |
| | | | 1H), 4.50 − 4.40 (m, |
| | | | 1H), 4.24 − 3.96 (m, |
| | | | 6H), 3.82 − 3.65 (m, |
| | | | 1H), 2.50 (s, 3H), |
| | | | 2.35 − 2.18 (m, 2H), |
| | | | 2.14 − 1.98 (m, 4H), |
| | | | 1.81 (t, J = 12.8 Hz, |
| | | | 1H), 1.70 − 1.58 (m, |
| | | | 3H), 1.57 − 1.42 (m, |
| | | | 1H), 1.34 − 1.26 (m, |
| | | | 3H), 1.07 − 0.99 (m, |
| | | | 3H), 0.96 − 0.86 (m, |
| | | | 3H) |
| | 1002 | 921.5 | ¹H NMR (400 MHz, |
| | | | CDCl₃) δ 8.32 (s, |
| | | | 1H), 8.22 (s, 1H), |
| | | | 8.18 − 8.09 (m, 1H), |
| | | | 8.02 − 7.92 (m, 2H), |
| | | | 7.89 (s, 1H), 7.77 (s, |
| | | | 1H), 7.68 (d, J = 8.8 |
| | | | Hz, 1H), 4.82 − 4.64 |
| | | | (m, 3H), 4.49 − 4.44 |
| | | | (m, 1H), 4.29 − 4.15 |
| | | | (m, 4H), 3.95 − 3.85 |
| | | | (m, 1H), 3.20 − 2.99 |
| | | | (m, 4H), 2.40 − 2.15 |
| | | | (m, 4H), 2.05 − 1.97 |
| | | | (m, 2H), 1.91 (d, J = |
| | | | 11.6 Hz, 2H), 1.61 − |
| | | | 1.47 (m, 2H), 1.19 |
| | | | (d, J = 2.4 Hz, 18H) |
| | 1003 | 812.0 | ¹H NMR (400 MHz, |
| | | | CDCl₃) δ 8.64 − 8.55 |
| | | | (m, 2H), 7.98 − 7.70 |
| | | | (m, 4H), 7.69 − 7.28 |
| | | | (m, 5H), 7.24 (s, 1H), |
| | | | 7.19 − 7.07 (m, 2H), |
| | | | 4.73 − 4.61 (m, 2H), |
| | | | 4.59 − 4.47 (m, 1H), |
| | | | 4.25 − 4.18 (m, 1H), |
| | | | 4.17 − 4.03 (m, 3H), |
| | | | 4.01 − 3.89 (m, 2H), |
| | | | 3.67 − 3.41 (m, 2H), |
| | | | 3.38 − 2.74 (m, 2H), |
| | | | 2.30 − 2.19 (m, 2H), |
| | | | 1.96 (t, J = 10.4 Hz, |
| | | | 2H), 1.91 − 1.79 (m, |
| | | | 4H), 1.76 − 1.68 (m, |
| | | | 2H), 1.66 − 1.49 (m, |
| | | | 6H), 1.47 − 1.23 (m, |
| | | | 4H), 0.93 (m, 3H) |
| | 1004 | 836.1 | ¹H NMR (400 MHz, |
| | | | CDCl₃) δ 8.92 ( s, |
| | | | 1H), 8.67 ( s, 1H), |
| | | | 8.48 − 8.09 (m, 1H), |
| | | | 7.95 − 7.68 (m, 6H), |
| | | | 7.64 − 7.58 (m, 1H), |
| | | | 7.54 − 7.40 (m, 3H), |
| | | | 7.39 − 7.28 (m, 2H), |
| | | | 4.81 − 4.66 (m, 3H), |
| | | | 4.56 − 4.45 (m, 2H), |
| | | | 4.26 − 4.11 (m, 2H), |
| | | | 3.97 − 3.89 (m, 3H), |
| | | | 3.67 − 3.53 (m, 3H), |
| | | | 2.34 − 2.14 (m, 2H), |
| | | | 2.13 − 2.00 (m, 4H), |
| | | | 1.99 − 1.72 (m, 4H), |
| | | | 1.54 − 1.40 (m, 2H), |
| | | | 1.34 − 1.21 (m, 2H), |
| | | | 1.20 − 1.14 (m, 2H), |
| | | | 1.07 − 0.99 (m, 1H), |
| | | | 0.92 − 0.83 (m, 3H) |
| | 1005 | 935.5 | ¹H NMR (400 MHz, |
| | | | CDCl₃) δ 8.34 (s, |
| | | | 1H), 8.28 (s, 1H), |
| | | | 8.03 (s, 1H), 7.95 (s, |
| | | | 1H), 7.90 (d, J = 8.8 |
| | | | Hz, 1H), 7.83 (s, |
| | | | 1H), 7.61 (d, J = 8.8 |
| | | | Hz, 1H), 5.24 − 5.11 |
| | | | (m, 1H), 4.82 − 4.80 |
| | | | (m, 1H), 4.58 (t, J = |
| | | | 8.4 Hz, 1H), 4.41 − |
| | | | 4.33 (m, 1H), 4.30 − |
| | | | 4.08 (m, 5H), 3.18 − |
| | | | 3.04 (m, 4H), 2.42 − |
| | | | 2.30 (m, 1H), 2.28 − |
| | | | 2.14 (m, 2H), 2.12 − |
| | | | 1.98 (m, 2H), 1.98 − |
| | | | 1.90 (m, 2H), 1.75 − |
| | | | 1.49 (m, 5H), 1.20 |
| | | | (d, J = 11.6 Hz, 18H) |
| | 1006 | 975.1 | ¹H NMR (400 MHz, |
| | | | CDCl₃) δ 8.50 − 8.71 |
| | | | (m, 2H) 8.11 |
| | | | (d, J = 8.00 Hz, 1H) |
| | | | 7.82 − 7.94 (m, 2H) |
| | | | 7.62 − 7.73 (m, 2H) |
| | | | 7.24 − 7.38 (m, 11H) |
| | | | 4.52 − 5.04 (m, 4H) |
| | | | 4.14 − 4.46 (m, 6H) |
| | | | 3.89 − 4.08 (m, 2H) |
| | | | 3.58 − 3.69 (m, 4H) |
| | | | 2.89 − 3.18 (m, 4H) |
| | | | 2.15 − 2.31 (m, 2H) |
| | | | 1.90 − 2.12 (m, 6H) |
| | | | 1.71 − 1.84 (m, 2H) |
| | 1007 | 916.1 | ¹H NMR (400 MHz, |
| | | | ACETONITRILE- |
| | | | d3) δ 8.19 − 8.12 (m, |
| | | | 1H), 8.08 − 7.95 (m, |
| | | | 2H), 7.82 − 7.75 (m, |
| | | | 0.6H), 7.73 − 7.67 |
| | | | (m, 0.4H), 7.67 − |
| | | | 7.61 (m, 1H), 7.60 − |
| | | | 7.55 (m, 1H), 7.54 − |
| | | | 7.43 (m, 3H), 7.43 − |
| | | | 7.28 (m, 5H), 5.09 − |
| | | | 4.99 (m, 1H), 4.64 − |
| | | | 4.55 (m, 1H), 4.41 − |
| | | | 4.21 (m, 4H), 4.10 − |
| | | | 4.02 (m, 0.6H), 4.02 − |
| | | | 3.94 (m, 0.4H), |
| | | | 3.82 − 3.75 (m, |
| | | | 0.4H), 3.74 − 3.32 |
| | | | (m, 3.6H), 3.17 − |
| | | | 3.09 (m, 2H), 2.42 − |
| | | | 2.36 (m, 2H), 2.35 − |
| | | | 2.17 (m, 2H), 2.12 − |
| | | | 1.97 (m, 5H), 1.90 − |
| | | | 1.52 (m, 6H), 0.88 |
| | | | (d, J = 6.6 Hz, 6H). |
| | 1008 | 864.1 | ¹H NMR (400 MHz, |
| | | | ACETONITRILE- |
| | | | d3) δ 8.61 − 8.54 (m, |
| | | | 1H), 8.50 − 8.45 (m, |
| | | | 1H), 8.24 − 8.17 (m, |
| | | | 1H), 8.11 − 8.05 (m, |
| | | | 1H), 8.04 − 7.99 (m, |
| | | | 1H), 7.92 − 7.87 (m, |
| | | | 0.5H), 7.86 − 7.76 |
| | | | (m, 1.5H), 7.70 − |
| | | | 7.64 (m, 1H), 7.61 − |
| | | | 7.55 (m, 1H), 7.55 − |
| | | | 7.44 (m, 3H), 7.40 − |
| | | | 7.33 (m, 1H), 4.83 − |
| | | | 4.75 (m, 0.5H), 4.68 − |
| | | | 4.58 (m, 1.5H), |
| | | | 4.58 − 4.49 (m, 1H), |
| | | | 4.49 − 4.43 (m, |
| | | | 0.5H), 4.40 − 4.24 |
| | | | (m, 3H), 4.23 − 4.16 |
| | | | (m, 0.5H), 4.02 − |
| | | | 3.87 (m, 3H), 3.16 − |
| | | | 3.09 (m, 2H), 2.41 − |
| | | | 2.35 (m, 2H), 2.10 − |
| | | | 1.99 (m, 5H), 1.91 − |
| | | | 1.74 (m, 5H), 1.69 − |
| | | | 1.54 (m, 1H), 0.91 − |
| | | | 0.85 (m, 6H). |
| | 1009 | 879.1 | ¹H NMR (400 MHz, |
| | | | CD₃OD) δ 8.62 − |
| | | | 8.53 (m, 1H), 8.45 |
| | | | (dd, J = 11.7, 4.4 Hz, |
| | | | 1H), 8.21 − 8.19 (m, |
| | | | 2H), 8.14 − 7.94 (m, |
| | | | 2H), 7.69 − 7.63 (m, |
| | | | 1H), 7.52 − 7.39 (m, |
| | | | 1H), 4.99 − 4.88 (m, |
| | | | 1H), 4.80 − 4.68 (m, |
| | | | 1H), 4.63 − 4.47 (m, |
| | | | 2H), 4.45 − 4.15 (m, |
| | | | 5H), 4.13 − 3.96 (m, |
| | | | 3H), 3.22 − 3.10 (m, |
| | | | 4H), 2.80 − 2.65 (m, |
| | | | 2H), 2.38 − 2.25 (m, |
| | | | 1H), 2.21 − 1.79 (m, |
| | | | 9H), 1.18 − 1.11 (m, |
| | | | 12H). |
| | 1010 | 952.0 | ¹H NMR (400 MHz, |
| | | | CD₃OD) δ 8.23 − |
| | | | 8.18 (m, 1H), 8.18 − |
| | | | 8.13 (m, 1H), 8.12 − |
| | | | 8.05 (m, 1H), 7.69 − |
| | | | 7.61 (m, 1H), 7.45 |
| | | | (dd, J = 18.3, 6.8 Hz, |
| | | | 1H), 6.65 − 6.56 (m, |
| | | | 1H), 6.53 − 6.43 (m, |
| | | | 1H), 5.09 − 4.99 (m, |
| | | | 1H), 4.67 − 4.58 (m, |
| | | | 1H), 4.49 − 4.39 (m, |
| | | | 1H), 4.35 − 4.13 (m, |
| | | | 5H), 4.13 − 4.05 (m, |
| | | | 5H), 3.96 − 3.86 (m, |
| | | | 1H), 3.79 − 3.48 (m, |
| | | | 3H), 3.47 − 3.35 (m, |
| | | | 6H), 3.26 − 3.12 (m, |
| | | | 4H), 2.41 − 2.18 (m, |
| | | | 2H), 2.13 − 1.92 (m, |
| | | | 6H), 1.92 − 1.75 (m, |
| | | | 2H), 1.75 − 1.58 (m, |
| | | | 2H). |
| | 1011 | 883.0 | ¹H NMR (400 MHz, |
| | | | CD₃OD) δ 8.60 − |
| | | | 8.52 (m, 1H), 8.49 − |
| | | | 8.41 (m, 1H), 8.23 − |
| | | | 8.16 (m, 2H), 8.13 − |
| | | | 7.92 (m, 2H), 7.70 − |
| | | | 7.61 (m, 1H), 7.52 − |
| | | | 7.39 (m, 1H), 5.00 − |
| | | | 4.91 and 4.74 − 4.67 |
| | | | (m, 1H), 4.81 − 4.75 |
| | | | (m, 1H), 4.64 − 4.50 |
| | | | (m, 2H), 4.45 − 4.17 |
| | | | (m, 5H), 4.13 − 3.99 |
| | | | (m, 7H), 3.50 − 3.38 |
| | | | (m, 6H), 3.27 − 3.12 |
| | | | (m, 4H), 2.39 − 2.25 |
| | | | (m, 1H), 2.22 − 1.78 |
| | | | (m, 9H). |
| | 1012 | 931.1 | ¹H NMR (400 MHz, |
| | | | ACETONITRILE- |
| | | | d3) δ 8.17 − 8.09 (m, |
| | | | 1H), 8.08 − 8.01 (m, |
| | | | 1H), 7.99 (s, 1H), |
| | | | 7.74 − 7.55 (m, 2H), |
| | | | 7.45 − 7.21 (m, 5H), |
| | | | 5.72 − 5.49 (m, 2H), |
| | | | 5.11 − 4.96 (m, 1H), |
| | | | 4.94 − 4.77 (m, 1H), |
| | | | 4.59 (t, J = 8.4 Hz, |
| | | | 1H), 4.44 − 4.23 (m, |
| | | | 2H), 4.22 − 3.92 (m, |
| | | | 3H), 3.85 − 3.30 (m, |
| | | | 4H), 3.07 (t, J = 6.5 |
| | | | Hz, 2H), 2.38 − 2.19 |
| | | | (m, 1H), 2.13 − 1.95 |
| | | | (m, 5H), 1.92 − 1.85 |
| | | | (m, 1H), 1.83 − 1.56 |
| | | | (m, 5H), 1.27 (d, J = |
| | | | 6.1 Hz, 6H), 1.16 (s, |
| | | | 9H). |
| | 1013 | 1034.1 | ¹H NMR (400 MHz, |
| | | | CD₃OD) δ 8.59 (s, |
| | | | 1H), 8.29 (br. s., 1H), |
| | | | 8.10 (d, J = 8.3 Hz, |
| | | | 1H), 8.06 (d, J = 2.4 |
| | | | Hz, 2H), 7.77 (d, J = |
| | | | 9.0 Hz, 1H), 7.46 (t, |
| | | | J = 7.6 Hz, 1H), 6.58 |
| | | | (d, J = 9.5 Hz, 1H), |
| | | | 6.51 − 6.43 (m, 1H), |
| | | | 5.14 − 5.06 (m, 1H), |
| | | | 4.73 − 4.65 (m, 1H), |
| | | | 4.57 − 4.51 (m, |
| | | | 0.5H), 4.51 − 4.45 |
| | | | (m, 1H), 4.35 − 4.21 |
| | | | (m, 4H), 4.19 (s, 4H), |
| | | | 4.08 − 4.02 (m, |
| | | | 0.5H), 3.97 − 3.91 |
| | | | (m, 0.5H), 3.89 − |
| | | | 3.82 (m, 0.5H), 3.75 − |
| | | | 3.63 (m, 6H), 3.62 − |
| | | | 3.51 (m, 6H), 3.35 (s, |
| | | | 6H), 3.26 − 3.13 (m, |
| | | | 4H), 2.41 − 2.19 (m, |
| | | | 2H), 2.13 − 1.94 (m, |
| | | | 6H), 1.93 − 1.76 (m, |
| | | | 2H), 1.76 − 1.60 (m, |
| | | | 2H). |
| | 1014 | 922.08 | ¹H NMR (400 MHz, |
| | | | CDCl₃) δ 8.08 (s, |
| | | | 1H), 7.92 (d, J = 8.7 |
| | | | Hz, 1H), 7.84 (s, |
| | | | 1H), 7.63 (d, J = 8.6 |
| | | | Hz, 2H), 4.63 (s, |
| | | | 1H), 4.43 (d, J = 9.4 |
| | | | Hz, 2H), 4.17 (dt, J = |
| | | | 19.8, 6.9 Hz, 4H), |
| | | | 4.02 − 3.78 (m, 2H), |
| | | | 3.50 − 3.26 (m, 3H), |
| | | | 3.08 (q, J = 6.2 Hz, |
| | | | 3H), 2.95 (d, J = 7.4 |
| | | | Hz, 3H), 2.28 − 2.13 |
| | | | (m, 2H), 1.94 (d, J = |
| | | | 33.2 Hz, 5H), 1.78 (s, |
| | | | 1H), 1.64 (d, J = 12.4 |
| | | | Hz, 6H), 1.20 (s, |
| | | | 18H). |
| | 1015 | 965.15 | ¹H NMR (400 MHz, |
| | | | CDCl₃) δ 8.08 (s, |
| | | | 1H), 7.92 (d, J = 8.4 |
| | | | Hz, 1H), 7.89 − 7.78 |
| | | | (m, 1H), 7.68 − 7.52 |
| | | | (m, 2H), 4.93 − 4.57 |
| | | | (m, 2H), 4.44 (t, J = |
| | | | 19.6 Hz, 2H), 4.16 |
| | | | (d, J = 24.1 Hz, 4H), |
| | | | 4.06 − 3.79 (m, 2H), |
| | | | 3.58 − 3.46 (m, 3H), |
| | | | 3.09 (s, 7H), 2.95 − |
| | | | 2.76 (m, 3H), 2.28 − |
| | | | 2.12 (m, 2H), 2.01 (s, |
| | | | 4H), 1.90 (s, 2H), |
| | | | 1.77 (s, 5H), 1.20 (q, |
| | | | J = 3.5 Hz, 18H). |
| | 1016 | 786.3 | ¹H NMR (400 MHz, |
| | | | CDCl₃) δ 9.01 − 8.80 |
| | | | (m, 1H), 8.78 − 8.66 |
| | | | (m, 1H), 8.49 (d, J = |
| | | | 7.2 Hz, 1H), 7.89 − |
| | | | 7.76 (m, 4H), 7.75 − |
| | | | 7.61 (m, 1H), 7.49 − |
| | | | 7.38 (m, 1H), 7.32 − |
| | | | 7.28 (m, 1H), 7.17 − |
| | | | 7.09 (m, 3H), 4.74 (s, |
| | | | 3H), 4.57 − 4.44 (m, |
| | | | 2H), 4.28 − 4.14 (m, |
| | | | 1H), 3.97 (m, 3H), |
| | | | 3.57 − 3.17 (m, 3H), |
| | | | 2.36 − 2.23 (m, 1H), |
| | | | 2.23 − 2.14 (m, 1H),, |
| | | | 2.12 − 1.99 (m, 4H), |
| | | | 1.97 − 1.86 (m, 2H), |
| | | | 1.86 − 1.66 (m, 2H), |
| | | | 1.57 − 1.43 (m, 2H), |
| | | | 1.22 − 1.16 (m, 2H), |
| | | | 1.16 − 1.06 (m, 4H), |
| | | | 0.88 − 0.79 (m, 3H) |
| | 1017 | 845.3 | ¹H NMR (400 MHz, |
| | | | CD₃OD) δ 8.40 − |
| | | | 8.33 (m, 1H), 8.16 − |
| | | | 8.06 (m, 1H), 8.05 − |
| | | | 7.98 (m, 1H), 7.94 − |
| | | | 7.84 (m, 2H), 7.53 − |
| | | | 7.46 (m, 1H), 7.36 − |
| | | | 7.28 (m, 2H), 7.21 − |
| | | | 7.14 (m, 3H), 7.10 |
| | | | (dd, J = 7.2, 18.4 Hz, |
| | | | 1H), 4.78 − 4.67 (m, |
| | | | 2H), 4.58 − 4.49 (m, |
| | | | 2H), 4.49 − 4.40 (m, |
| | | | 1H), 4.37 − 4.29 (m, |
| | | | 1H), 4.12 − 4.04 (m, |
| | | | 2H), 4.02 − 3.89 (m, |
| | | | 3H), 3.61 (s, 1H), |
| | | | 3.58 − 3.49 (m, 2H), |
| | | | 3.39 − 3.33 (m, 1H), |
| | | | 3.17 − 3.14 (m, 9H), |
| | | | 2.37 − 2.26 (m, 1H), |
| | | | 2.20 − 2.06 (m, 2H), |
| | | | 2.04 − 1.90 (m, 4H), |
| | | | 1.89 − 1.78 (m, 3H), |
| | | | 1.62 − 1.48 (m, 2H), |
| | | | 0.92 − 0.80 (m, 3H) |
| | 1018 | 890.3 | ¹H NMR (400 MHz, |
| | | | CDCl₃) δ 8.64 − 8.51 |
| | | | (m, 1H), 8.46 − 8.30 |
| | | | (m, 1H), 8.10 − 7.78 |
| | | | (m, 3H), 7.65 − 7.52 |
| | | | (m, 1H), 7.39 − 7.29 |
| | | | (m, 1H), 7.27 − 7.03 |
| | | | (m, 4H), 6.03-5.89 |
| | | | (m, 1H), 5.08 − 4.63 |
| | | | (m, 4H), 4.60 − 4.42 |
| | | | (m, 3H), 4.35 − 4.20 |
| | | | (m, 2H), 4.15 − 4.10 |
| | | | (m, 1H), 4.03 (t, J = |
| | | | 6.4 Hz, 1H), 3.98 − |
| | | | 3.90 (m, 2H), 3.88 − |
| | | | 3.77 (m, 1H), 3.76 − |
| | | | 3.65 (m, 1H), 3.49 (t, |
| | | | J = 10.8 Hz, 1H), |
| | | | 3.45 − 3.27 (m, 3H), |
| | | | 2.65 − 2.42 (m, 2H), |
| | | | 2.31 − 2.12 (m, 2H), |
| | | | 2.04 (d, J = 4.8 Hz, |
| | | | 2H), 1.99 − 1.87 (m, |
| | | | 2H), 1.82 (s, 2H), |
| | | | 1.66 − 1.58 (m, 1H), |
| | | | 1.57 − 1.46 (m, 1H), |
| | | | 1.43 (d, J = 6.9 Hz, |
| | | | 2H), 1.38 (d, J = 6.4 |
| | | | Hz, 1H), 1.30 − 1.25 |
| | | | (m, 2H), 1.02 − 0.72 |
| | | | (m, 3H) |
| | 1019 | 828.6 | ¹H NMR (400 MHz, |
| | | | CD₃OD) δ 8.52 (dd, |
| | | | J = 0.8, 20.0 Hz, 1H), |
| | | | 8.26 (d, J = 4.4 Hz, |
| | | | 1H), 8.04 − 8.01 (m, |
| | | | 1H), 7.93 − 7.86 (m, |
| | | | 2H), 7.52 − 7.44 (m, |
| | | | 1H), 7.35 − 7.29 (m, |
| | | | 2H), 7.20 − 7.12 (m, |
| | | | 3H), 4.81 (s, 1H), |
| | | | 4.74 (dd, J = 3.2, 10.8 |
| | | | Hz, 1H), 4.61 − 4.51 |
| | | | (m, 5H), 4.41 − 4.27 |
| | | | (m, 2H), 4.11 − 4.04 |
| | | | (m, 3H), 3.97 − 3.90 |
| | | | (m, 2H), 3.89 − 3.82 |
| | | | (m, 1H), 3.58 − 3.47 |
| | | | (m, 2H), 2.54 − 2.45 |
| | | | (m, 2H), 2.35 − 2.27 |
| | | | (m, 1H), 2.18 − 2.07 |
| | | | (m, 2H), 2.05 − 1.83 |
| | | | (m, 7H), 1.58 − 1.50 |
| | | | (m, 2H), 1.15 − 1.07 |
| | | | (m, 3H), 0.90 − 0.83 |
| | | | (m, 3H) |
| | 1020 | 883.3 | ¹H NMR (400 MHz, |
| | | | CD₃OD) δ 8.68 − |
| | | | 8.57 (m, 1H), 8.18 − |
| | | | 7.91 (m, 4H), 7.80 ( |
| | | | d, J = 7.4 Hz, 1H), |
| | | | 7.71 − 7.59 (m, 1H), |
| | | | 7.52 − 7.41 (m, 1H), |
| | | | 7.40 − 7.29 (m, 2H), |
| | | | 7.27 − 7.13 (m, 3H), |
| | | | 6.20 − 5.75 (m, 2H), |
| | | | 5.06 − 4.94 (m, 2H), |
| | | | 4.73 (s, 1H), 4.50 − |
| | | | 4.14 (m, 4H), 4.01 − |
| | | | 3.76 (m, 3H), 3.05 − |
| | | | 2.86 (m, 2H), 2.35 − |
| | | | 2.18 (m, 2H), 2.11 − |
| | | | 1.78 (m, 8H), 1.72 − |
| | | | 1.47 (m, 4H), 1.29 − |
| | | | 1.18 (m, 2H), 1.06 |
| | | | (d, J = 7.2 Hz, 1H), |
| | | | 0.95 − 0.81 (m, 3H) |
| | 1021 | 829.5 | ¹H NMR (400 MHz, |
| | | | CDCl₃) δ 8.68 − 8.28 |
| | | | (m, 2H), 7.96 − 7.63 |
| | | | (m, 4H), 7.51 − 7.37 |
| | | | (m, 1H), 7.33 − 7.27 |
| | | | (m, 2H), 7.20 − 7.08 |
| | | | (m, 3H), 6.89 − 6.82 |
| | | | (m, 1H), 5.05 − 4.79 |
| | | | (m, 1H), 4.77 − 4.54 |
| | | | (m, 3H), 4.48 − 4.26 |
| | | | (m, 1H), 4.24 − 4.08 |
| | | | (m, 2H), 4.02 − 3.90 |
| | | | (m, 4H), 3.50 − 3.38 |
| | | | (m, 2H), 3.35 − 3.08 |
| | | | (m, 1H), 2.93 − 2.83 |
| | | | (m, 6H), 2.33 − 2.25 |
| | | | (m, 1H), 2.18 − 1.99 |
| | | | (m, 5H), 1.82 − 1.65 |
| | | | (m, 2H), 1.63 − 1.47 |
| | | | (m, 3H), 1.20 − 1.12 |
| | | | (m, 3H), 1.05 − 0.99 |
| | | | (m, 3H), 0.95 − 0.85 |
| | | | (m, 3H) |
| | 1022 | 851.5 | ¹H NMR (400 MHz, |
| | | | CDCl₃) δ 8.83 − 8.50 |
| | | | (m, 1H), 8.47 − 8.33 |
| | | | (m, 1H), 8.04 − 7.79 |
| | | | (m, 3H), 7.67 − 7.59 |
| | | | (m, 1H), 7.59 − 7.46 |
| | | | (m, 1H), 7.39 − 7.29 |
| | | | (m, 1H), 7.25 − 7.17 |
| | | | (m, 1H), 7.15 − 7.04 |
| | | | (m, 2H), 6.08 − 5.85 |
| | | | (m, 1H), 5.11 − 4.92 |
| | | | (m, 1H), 4.86 − 4.63 |
| | | | (m, 2H), 4.53 (t, J = |
| | | | 5.6 Hz, 1H), 4.48 − |
| | | | 4.28 (m, 1H), 4.21 − |
| | | | 4.09 (m, 1H), 4.08 − |
| | | | 4.03 (m, 2H), 4.02 − |
| | | | 3.97 (m, 2H), 3.65 − |
| | | | 3.53 (m, 2H), 3.12 − |
| | | | 3.05 (m, 6H), 2.35 − |
| | | | 2.25 (m, 1H), 2.24 − |
| | | | 2.15 (m, 1H), 2.13 − |
| | | | 1.97 (m, 4H), 1.96 − |
| | | | 1.90 (m, 1H), 1.85 |
| | | | (d, J = 6.4 Hz, 2H), |
| | | | 1.81 − 1.66 (m, 1H), |
| | | | 1.66 − 1.55 (m, 2H), |
| | | | 1.31 − 1.12 (m, 3H), |
| | | | 0.95 − 0.87 (m, 3H) |
| | 1023 | 820.2 | ¹H NMR (400 MHz, |
| | | | CD₃OD) δ 8.61 (m, |
| | | | 2H), 8.12 − 8.04 (m, |
| | | | 2H), 7.97 (m, 1H), |
| | | | 7.67 − 7.54 (m, 2H), |
| | | | 7.33 (m, 2H), 7.24 − |
| | | | 7.15 (m, 3H), 6.19 − |
| | | | 5.96 (m, 1H), 4.95 − |
| | | | 4.90 (m, 1H), 4.78 − |
| | | | 4.71 (m, 1H), 4.65 |
| | | | (m, 1H), 4.56 (m, |
| | | | 1H), 4.46 − 4.34 (m, |
| | | | 1H), 4.22 − 4.14 (m, |
| | | | 5H), 4.13 − 4.05 (m, |
| | | | 2H), 3.88 − 3.79 (m, |
| | | | 1H), 2.38 − 2.23 (m, |
| | | | 1H), 2.08 (m, 2H), |
| | | | 2.06 − 2.02 (m, 1H), |
| | | | 2.00 − 1.97 (m, 1H), |
| | | | 1.90 (s, 2H), 1.88 − |
| | | | 1.79 (m, 3H), 1.24 − |
| | | | 1.21 (m, 3H), 1.17 (t, |
| | | | J = 8.00 Hz, 6H) |
| | 1024 | 845.4 | ¹H NMR (400 MHz, |
| | | | CD₃OD) δ 8.32 − |
| | | | 8.21 (m, 1H), 8.15 − |
| | | | 8.06 (m, 2H), 8.04 − |
| | | | 7.93 (m, 2H), 7.65 |
| | | | (d, J = 8.0 Hz, 1H), |
| | | | 7.40 − 7.29 (m, 2H), |
| | | | 7.26 − 7.13 (m, 3H), |
| | | | 6.57 − 6.46 (m, 1H), |
| | | | 6.19 − 5.97 (m, 1H), |
| | | | 4.78 − 4.72 (m, 1H), |
| | | | 4.66 − 4.52 (m, 3H), |
| | | | 4.51 − 4.41 (m, 3H), |
| | | | 4.41 − 4.29 (m, 2H), |
| | | | 4.23 − 4.14 (m, 1H), |
| | | | 4.13 − 4.04 (m, 2H), |
| | | | 3.97 (t, J = 6.8 Hz, |
| | | | 2H), 3.87 − 3.74 (m, |
| | | | 1H), 2.58 − 2.43 (m, |
| | | | 2H), 2.38 − 2.26 (m, |
| | | | 1H), 2.20 − 2.07 (m, |
| | | | 2H), 2.06 − 1.97 (m, |
| | | | 2H), 1.97 − 1.94 (m, |
| | | | 2H), 1.90 − 1.78 (m, |
| | | | 3H), 1.61 − 1.52 (m, |
| | | | 2H), 1.09 − 1.07 (m, |
| | | | 3H), 0.88 (t, J = 7.2 |
| | | | Hz, 3H) |
| | 1025 | 821.3 | ¹H NMR (400 MHz, |
| | | | CDCl₃) δ 8.76 − 8.49 |
| | | | (m, 1H), 8.31 − 8.22 |
| | | | (m, 1H), 7.92 (d, J = |
| | | | 4.0 Hz, 1H), 7.78 (d, |
| | | | J = 8.4 Hz, 1H), 7.76 − |
| | | | 7.61 (m, 1H), 7.41 − |
| | | | 7.29 (m, 1H), 6.94 − |
| | | | 6.84 (m, 1H), 4.92 − |
| | | | 4.79 (m, 1H), 4.77 − |
| | | | 4.60 (m, 2H), 4.52 |
| | | | (d, J = 5.2 Hz, 1H), |
| | | | 4.47 − 4.33 (m, 2H), |
| | | | 4.29 − 4.24 (m, 1H), |
| | | | 4.22 (s, 1H), 4.11 − |
| | | | 4.05 (m, 1H), 4.04 − |
| | | | 3.87 (m, 4H), 3.54 − |
| | | | 3.42 (m, 1H), 3.40 − |
| | | | 3.24 (m, 2H), 3.14 (s, |
| | | | 3H), 3.09 (s, 3H), |
| | | | 2.35 − 2.24 (m, 1H), |
| | | | 2.22 − 2.14 (m, 1H), |
| | | | 2.13 − 2.05 (m, 2H), |
| | | | 2.04 − 1.98 (m, 2H), |
| | | | 1.95 − 1.74 (m, 4H), |
| | | | 1.67 − 1.58 (m, 2H), |
| | | | 1.37 − 1.24 (m, 3H), |
| | | | 0.91 (m, 3H) |
| | 1026 | 837.2 | ¹H NMR (400 MHz, |
| | | | CD₃OD) δ 8.41 − |
| | | | 8.28 (m, 1H), 8.18 |
| | | | (d, J = 8.4 Hz, 1H), |
| | | | 8.12 − 8.04 (m, 2H), |
| | | | 8.02 − 7.95 (m, 1H), |
| | | | 7.71 − 7.61 (m, 1H), |
| | | | 7.39 − 7.29 (m, 2H), |
| | | | 7.23 − 7.13 (m, 3H), |
| | | | 6.19 − 6.01 (m, 1H), |
| | | | 5.00 − 4.90 (m, 1H), |
| | | | 4.80 − 4.67 (m, 2H), |
| | | | 4.63 − 4.48 (m, 2H), |
| | | | 4.46 − 4.28 (m, 1H), |
| | | | 4.13 − 4.00 (m, 2H), |
| | | | 3.98 − 3.86 (m, 2H), |
| | | | 3.85 − 3.79 (m, 1H), |
| | | | 3.29 (d, J = 7.2 Hz, |
| | | | 3H), 2.38 − 2.25 (m, |
| | | | 1H), 2.18 − 2.05 (m, |
| | | | 2H), 2.03 − 1.75 (m, |
| | | | 7H), 1.62 − 1.45 (m, |
| | | | 2H), 1.29 − 1.18 (m, |
| | | | 3H), 0.97 − 0.80 (m, |
| | | | 3H) |
| | 1027 | 804.3 | ¹H NMR (400 MHz, |
| | | | CD₃OD) δ 8.86 − |
| | | | 8.80 (m, 1H), 8.62 − |
| | | | 8.52 (m, 1H), 8.15 − |
| | | | 8.05 (m, 2H), 8.00 |
| | | | (d, J = 4.4, 8.3 Hz, |
| | | | 1H), 7.89 − 7.79 (m, |
| | | | 1H), 7.67 − 7.61 (m, |
| | | | 1H), 7.40 − 7.30 (m, |
| | | | 2H), 7.26 − 7.16 (m, |
| | | | 3H), 6.10 (s, 1H), |
| | | | 4.79 − 4.72 (m, 2H), |
| | | | 4.71 − 4.63 (m, 1H), |
| | | | 4.61 − 4.48 (m, 2H), |
| | | | 4.47 − 4.31 (m, 2H), |
| | | | 4.21 − 4.07 (m, 2H), |
| | | | 4.00 − 3.96 (m, 1H), |
| | | | 3.92 − 3.82 (m, 1H), |
| | | | 2.59 − 2.51 (m, 3H), |
| | | | 2.38 − 2.26 (m, 1H), |
| | | | 2.22 − 2.09 (m, 2H), |
| | | | 2.08 − 2.00 (m, 2H), |
| | | | 2.00 − 1.93 (m, 2H), |
| | | | 1.92 − 1.85 (m, 2H), |
| | | | 1.85 − 1.79 (m, 1H), |
| | | | 1.63 − 1.53 (m, 2H), |
| | | | 1.28 − 1.20 (m, 3H), |
| | | | 0.95 − 0.85 (m, 3H) |
| | 1028 | 845.2 | ¹H NMR (400 MHz, |
| | | | CDCl₃) δ 8.68 − 8.39 |
| | | | (m, 1H), 7.89 − 7.80 |
| | | | (m, 2H), 8.15 − 7.78 |
| | | | (m, 2H), 7.67 − 7.56 |
| | | | (m, 1H), 7.26 (s, 2H), |
| | | | 7.17 − 7.01 (m, 3H), |
| | | | 6.32 − 6.10 (m, 1H), |
| | | | 6.08 − 5.90 (m, 1H), |
| | | | 5.05 − 4.86 (m, 1H), |
| | | | 4.83 − 4.65 (m, 1H), |
| | | | 4.59 − 4.46 (m, 2H), |
| | | | 4.45 − 4.19 (m, 5H), |
| | | | 4.11 − 4.00 (m, 3H), |
| | | | 4.00 − 3.92 (m, 2H), |
| | | | 3.84 (s, 1H), 2.54 (td, |
| | | | J = 8.1, 16.7 Hz, 2H), |
| | | | 2.33 − 2.14 (m, 2H), |
| | | | 2.04 (s, 3H), 2.00 − |
| | | | 1.88 (m, 2H), 1.87 − |
| | | | 1.66 (m, 3H), 1.63 − |
| | | | 1.55 (m, 2H), 1.33 − |
| | | | 1.29 (m, 3H), 0.95 − |
| | | | 0.84 (m, 3H) |
| | 1029 | 847.3 | ¹H NMR (400 MHz, |
| | | | CDCl₃) δ 8.77 − 8.56 |
| | | | (m, 1H), 8.30 − 7.98 |
| | | | (m, 3H), 7.88 − 7.58 |
| | | | (m, 3H), 7.23 (s, 1H), |
| | | | 7.10 − 7.05 (m, 3H), |
| | | | 6.90 − 6.82 (m, 1H), |
| | | | 6.05 − 5.91 (m, 1H), |
| | | | 5.03 − 4.94 (m, 1H), |
| | | | 4.83 − 4.68 (m, 2H), |
| | | | 4.52 (t, J = 4.8 Hz, |
| | | | 1H), 4.43 − 4.34 (m, |
| | | | 1H), 4.04 − 3.95 (m, |
| | | | 6H), 3.69 − 3.57 (m, |
| | | | 1H), 3.12 (s, 3H), |
| | | | 3.06 (s, 3H), 2.30 − |
| | | | 2.28 (m, 1H), 2.10 − |
| | | | 2.01 (m, 5H), 1.63 − |
| | | | 1.57 (m, 5H), 1.28 (t, |
| | | | J = 7.2 Hz, 3H), 1.01 |
| | | | (d, J = 3.6 Hz, 3H), |
| | | | 0.90 (q, J = 6.8 Hz, |
| | | | 3H |
| | 1030 | 826.3 | ¹H NMR (400 MHz, |
| | | | CD₃OD) δ 8.09 − |
| | | | 8.05 (m, 2H), 8.01 − |
| | | | 7.96 (m, 1H), 7.67 − |
| | | | 7.62 (m, 1H), 7.36 − |
| | | | 7.31 (m, 2H), 7.22 − |
| | | | 7.15 (m, 3H), 6.95 (s, |
| | | | 2H), 6.19 − 5.99 (m, |
| | | | 1H), 5.33 − 5.14 (m, |
| | | | 1H), 4.72 (d, J = 11.2 |
| | | | Hz, 1H), 4.63 − 4.59 |
| | | | (m, 1H), 4.45 − 4.42 |
| | | | (m, 1H), 4.11 − 4.04 |
| | | | (m, 1H), 3.99 − 3.73 |
| | | | (m, 6H), 3.64 (d, J = |
| | | | 10.8 Hz, 1H), 2.34 − |
| | | | 2.23 (m, 1H), 2.15 − |
| | | | 1.82 (m, 9H), 1.60 − |
| | | | 1.48 (m, 2H), 1.25 − |
| | | | 1.19 (m, 3H), 0.91 − |
| | | | 0.83 (m, 3H) |
| | 1031 | 827.3 | ¹H NMR (400 MHz, |
| | | | CD₃OD) δ 8.26 (d, J = |
| | | | 9.2 Hz, 1H), 8.04 − |
| | | | 8.00 (m, 1H), 7.96 |
| | | | (d, J = 7.2 Hz, 1H), |
| | | | 7.92 − 7.84 (m, 2H), |
| | | | 7.53 − 7.46 (m, 1H), |
| | | | 7.35 − 7.28 (m, 2H), |
| | | | 7.20 − 7.13 (m, 3H), |
| | | | 6.51 (dd, J = 7.2, |
| | | | 10.8 Hz, 1H), 4.95 − |
| | | | 4.91 (m, 1H), 4.74 |
| | | | (d, J = 11.2 Hz, 1H), |
| | | | 4.62 − 4.52 (m, 3H), |
| | | | 4.49 − 4.37 (m, 3H), |
| | | | 4.35 − 4.30 (m, 1H), |
| | | | 4.21 − 4.14 (m, 1H), |
| | | | 4.10 − 4.05 (m, 2H), |
| | | | 3.95 − 3.90 (m, 2H), |
| | | | 3.89 − 3.81 (m, 1H), |
| | | | 3.57 − 3.46 (m, 2H), |
| | | | 2.56 − 2.46 (m, 2H), |
| | | | 2.34 − 2.27 (m, 1H), |
| | | | 2.15 − 2.06 (m, 2H), |
| | | | 2.01 − 1.82 (m, 7H), |
| | | | 1.58 − 1.50 (m, 2H), |
| | | | 1.14 (d, J = 7.2 Hz, |
| | | | 2H), 1.09 (dd, J = |
| | | | 4.8, 7.2 Hz, 1H), |
| | | | 0.89 − 0.84 (m, 3H) |
| | 1032 | 820.2 | ¹H NMR (400 MHz, |
| | | | CD₃OD) δ 8.72 − |
| | | | 8.60 (m, 2H), 8.13 − |
| | | | 8.03 (m, 2H), 8.01 − |
| | | | 7.95 (m, 1H), 7.69 − |
| | | | 7.55 (m, 2H), 7.39 − |
| | | | 7.30 (m, 2H), 7.22 − |
| | | | 7.13 (m, 3H), 6.19 − |
| | | | 6.02 (m, 1H), 4.78 − |
| | | | 4.71 (m, 1H), 4.66 |
| | | | (d, J = 7.2 Hz, 1H), |
| | | | 4.60 − 4.51 (m, 1H), |
| | | | 4.49 − 4.41 (m, 1H), |
| | | | 4.40 − 4.32 (m, 1H), |
| | | | 4.23 − 4.14 (m, 5H), |
| | | | 4.09 (d, J = 4.0 Hz, |
| | | | 1H), 3.97 − 3.88 (m, |
| | | | 1H), 3.86 − 3.77 (m, |
| | | | 2H), |
| | | | 2.38 − 2.26 (m, |
| | | | 1H), 2.20 − 2.06 (m, |
| | | | 2H), 2.05 − 1.96 (m, |
| | | | 2H), 1.95 − 1.89 (m, |
| | | | 2H), 1.88 − 1.77 (m, |
| | | | 3H), 1.57 − 1.45 (m, |
| | | | 2H), 1.21 (d, J = 7.2 |
| | | | Hz, 3H), 0.90 − 0.80 |
| | | | (m, 3H) |
| | 1033 | 862.2 | ¹H NMR (400 MHz, |
| | | | CDCl₃) δ 8.88 − 8.63 |
| | | | (m, 2H), 8.05 − 7.77 |
| | | | (m, 4H), 7.73 − 7.51 |
| | | | (m, 2H), 7.26 − 7.24 |
| | | | (m, 1H), 7.19 − 7.05 |
| | | | (m, 4H), 6.07 − 5.92 |
| | | | (m, 1H), 5.01 − 4.81 |
| | | | (m, 2H), 4.79 − 4.68 |
| | | | (m, 1H), 4.67 − 4.50 |
| | | | (m, 2H), 4.45 − 4.35 |
| | | | (m, 1H), 4.19 − 3.93 |
| | | | (m, 6H), 3.75 − 3.56 |
| | | | (m, 1H), 2.35 − 2.23 |
| | | | (m, 1H), 2.17 − 1.97 |
| | | | (m, 5H), 1.83 − 1.73 |
| | | | (m, 1H), 1.71 − 1.56 |
| | | | (m, 3H), 1.54 − 1.43 |
| | | | (m, 7H), 1.33 − 1.26 |
| | | | (m, 3H), 1.02 (t, J = |
| | | | 6.0 Hz, 3H), 0.95 − |
| | | | 0.86 (m, 3H) |
| | 1034 | 862.3 | ¹H NMR (400 MHz, |
| | | | CDCl₃) δ 8.92 − 8.57 |
| | | | (m, 2H), 8.09 − 7.77 |
| | | | (m, 4H), 7.70 − 7.46 |
| | | | (m, 2H), 7.39 − 7.31 |
| | | | (m, 2H), 7.23 − 7.08 |
| | | | (m, 2H), 6.04 − 5.84 |
| | | | (m, 1H), 5.01 − 4.82 |
| | | | (m, 2H), 4.79 − 4.69 |
| | | | (m, 1H), 4.66 − 4.52 |
| | | | (m, 2H), 4.44 − 4.34 |
| | | | (m, 1H), 4.16 − 3.95 |
| | | | (m, 6H), 3.80 − 3.65 |
| | | | (m, 1H), 2.35 − 2.24 |
| | | | (m, 1H), 2.17 − 1.97 |
| | | | (m, 5H), 1.82 − 1.73 |
| | | | (m, 1H), 1.71 − 1.57 |
| | | | (m, 3H), 1.48 (s, 6H), |
| | | | 1.45 − 1.38 (m, 1H), |
| | | | 1.13 (t, J = 6.8 Hz, |
| | | | 3H), 1.02 (t, J = 5.6 |
| | | | Hz, 3H), 0.95 − 0.83 |
| | | | (m, 3H) |
| | 1035 | 804.2 | ¹H NMR (400 MHz, |
| | | | CD₃OD) δ 8.87 − |
| | | | 8.80 (m, 1H), 8.64 − |
| | | | 8.53 (m, 1H), 8.14 − |
| | | | 8.04 (m, 2H), 7.98 |
| | | | (d, J = 5.2, 8.3 Hz, |
| | | | 1H), 7.90 − 7.82 (m, |
| | | | 1H), 7.70 − 7.62 (m, |
| | | | 1H), 7.40 − 7.13 (m, |
| | | | 5H), 6.21 − 6.02 (m, |
| | | | 1H), 5.01 ( d, J = 8.8 |
| | | | Hz, 1H), 4.79 − 4.71 |
| | | | (m, 2H), 4.70 − 4.62 |
| | | | (m, 1H), 4.61 − 4.52 |
| | | | (m, 2H), 4.50 − 4.41 |
| | | | (m, 1H), 4.40 − 4.31 |
| | | | (m, 1H), 4.17 (d, J = |
| | | | 6.8, 9.8 Hz, 1H), 4.14 − |
| | | | 4.06 (m, 1H), 3.88 − |
| | | | 3.79 (m, 2H), 2.60 − |
| | | | 2.52 (m, 3H), 2.37 − |
| | | | 2.28 (m, 1H), 2.22 − |
| | | | 2.14 (m, 1H), 2.14 − |
| | | | 2.06 (m, 2H), 2.02 |
| | | | (td, J = 5.8, 11.9 Hz, |
| | | | 2H), 1.97 − 1.91 (m, |
| | | | 2H), 1.90 − 1.85 (m, |
| | | | 2H), 1.84 − 1.79 (m, |
| | | | 2H), 1.57 − 1.46 (m, |
| | | | 2H), 1.21 (d, J = 7.1 |
| | | | Hz, 3H), 0.95 − 0.81 |
| | | | (m, 3H) |
| | 1036 | 818.4 | ¹H NMR (400 MHz, |
| | | | CDCl₃) δ 9.24 − 8.91 |
| | | | (m, 1H), 8.69 − 8.57 |
| | | | (m, 1H), 8.19 (d, J = |
| | | | 7.2 Hz, 1H), 8.00 (d, |
| | | | J = 6.0 Hz, 2H), 7.90 − |
| | | | 7.81 (m, 1H), 7.65 − |
| | | | 7.54 (m, 2H), 7.28 (s, |
| | | | 1H), 7.24 (s, 1H), |
| | | | 7.17 − 7.03 (m, 3H), |
| | | | 6.12 − 5.88 (m, 1H), |
| | | | 5.08 (d, J = 4.8 Hz, |
| | | | 1H), 4.82 (d, J = 7.6 |
| | | | Hz, 1H), 4.75 − 4.70 |
| | | | (m, 1H), 4.54 (t, J = |
| | | | 5.6 Hz, 1H), 4.47 (d, |
| | | | J = 8.8 Hz, 1H), 4.26 − |
| | | | 3.96 (m, 6H), 3.71 − |
| | | | 3.56 (m, 1H), 2.51 |
| | | | (d, J = 2.9 Hz, 3H), |
| | | | 2.36 − 2.26 (m, 1H), |
| | | | 2.22 − 1.98 (m, 5H), |
| | | | 1.85 − 1.76 (m, 1H), |
| | | | 1.71 − 1.45 (m, 4H), |
| | | | 1.32 − 1.26 (m, 3H), |
| | | | 1.02 (t, J = 5.6 Hz, |
| | | | 3H), 0.95 − 0.87 (m, |
| | | | 3H) |
| | 1037 | 818.2 | ¹H NMR (400 MHz, |
| | | | CDCl₃) δ 9.20 − 8.91 |
| | | | (m, 1H), 8.70 − 8.58 |
| | | | (m, 1H), 8.19 (d, J = |
| | | | 7.2 Hz, 1H), 8.06 − |
| | | | 7.92 (m, 2H), 7.89 − |
| | | | 7.82 (m, 1H), 7.67 − |
| | | | 7.48 (m, 3H), 7.38 − |
| | | | 7.31 (m, 2H), 7.25 − |
| | | | 7.16 (m, 2H), 6.02 − |
| | | | 5.86 (m, 1H), 5.12 − |
| | | | 5.04 (m, 1H), 4.82 |
| | | | (d, J = 8.0 Hz, 1H), |
| | | | 4.75 − 4.71 (m, 1H), |
| | | | 4.56 − 4.53 (m, 1H), |
| | | | 4.47 − 4.43 (m, 1H), |
| | | | 4.29 − 4.20 (m, 1H), |
| | | | 4.09 − 3.98 (m, 5H), |
| | | | 3.81 − 3.67 (m, 1H), |
| | | | 2.51 (s, 3H), 2.35 − |
| | | | 2.26 (m, 1H), 2.22 − |
| | | | 2.11 (m, 1H), 2.09 − |
| | | | 1.96 (m, 4H), 1.84 − |
| | | | 1.76 (m, 1H), 1.73 − |
| | | | 1.58 (m, 3H), 1.56 − |
| | | | 1.42 (m, 1H), 1.17 − |
| | | | 1.11 (m, 3H), 1.05 − |
| | | | 0.99 (m, 3H), 0.93 − |
| | | | 0.87 (m, 3H) |
| | 1038 | 857.3 | ¹H NMR (400 MHz, |
| | | | CD₃OD) δ 8.56 − |
| | | | 8.47 (m, 1H), 8.36 − |
| | | | 8.23 (m, 1H), 8.07 − |
| | | | 7.96 (m, 1H), 7.95 − |
| | | | 7.82 (m, 2H), 7.53 − |
| | | | 7.44 (m, 1H), 7.39 − |
| | | | 7.27 (m, 3H), 7.18 − |
| | | | 7.14 (m, 3H), 4.78 − |
| | | | 4.70 (m, 2H), 4.61 − |
| | | | 4.51 (m, 2H), 4.49 − |
| | | | 4.34 (m, 1H), 4.27 − |
| | | | 4.18 (m, 1H), 4.15 − |
| | | | 4.04 (m, 2H), 3.96 − |
| | | | 3.89 (m, 2H), 3.88 |
| | | | (d, J = 3.6 Hz, 1H), |
| | | | 3.84 (d, J = 4.4 Hz, |
| | | | 4H), 3.59 − 3.45 (m, |
| | | | 2H), 3.41 (d, J = 4.8 |
| | | | Hz, 4H), 2.38 − 2.25 |
| | | | (m, 1H), 2.20 − 2.05 |
| | | | (m, 2H), 2.04 − 1.91 |
| | | | (m, 4H), 1.89 − 1.78 |
| | | | (m, 3H), 1.57 − 1.50 |
| | | | (m, 2H), 1.16 − 1.06 |
| | | | (m, 3H), 0.90 − 0.83 |
| | | | (m, 3H) |
| | 1039 | 820.2 | ¹H NMR (400 MHz, |
| | | | CDCl₃) δ 8.83 − 8.70 |
| | | | (m, 1H), 8.70 − 8.64 |
| | | | (m, 1H), 8.10 − 7.98 |
| | | | (m, 1H), 7.96 (s, 1H), |
| | | | 7.92 − 7.82 (m, 1H), |
| | | | 7.82 − 7.69 (m, 1H), |
| | | | 7.56 − 7.47 (m, 1H), |
| | | | 7.39 − 7.31 (m, 2H), |
| | | | 7.26 − 7.08 (m, 3H), |
| | | | 6.05 − 5.85 (m, 1H), |
| | | | 5.02 − 4.94 (m, 1H), |
| | | | 4.86 − 4.78 (m, 1H), |
| | | | 4.77 − 4.72 (m, 1H), |
| | | | 4.71 − 4.62 (m, 1H), |
| | | | 4.61 − 4.56 (m, 1H), |
| | | | 4.55 − 4.48 (m, 1H), |
| | | | 4.41 − 4.30 (m, 1H), |
| | | | 4.22 − 4.13 (m, 1H), |
| | | | 4.09 (d, J = 18.1 Hz, |
| | | | 3H), 4.05 − 3.99 (m, |
| | | | 4H), 3.99 − 3.90 (m, |
| | | | 1H), 2.34 − 2.22 (m, |
| | | | 1H), 2.21 − 2.09 (m, |
| | | | 1H), 2.09 − 1.97 (m, |
| | | | 4H), 1.94 − 1.78 (m, |
| | | | 3H), 1.77 − 1.67 (m, |
| | | | 1H), 1.66 − 1.56 (m, |
| | | | 2H), 1.22 − 1.07 (m, |
| | | | 3H), 0.99 − 0.81 (m, |
| | | | 3H) |
| | 1040 | 834.2 | ¹H NMR (400 MHz, |
| | | | CDCl₃) δ 8.85 − 8.59 |
| | | | (m, 2H), 7.98 (s, 1H), |
| | | | 7.89 − 7.78 (m, 2H), |
| | | | 7.62 − 7.56 (m, 1H), |
| | | | 7.28 (d, J = 2.8 Hz, |
| | | | 1H), 7.24 − 7.23 (m, |
| | | | 2H), 7.13 − 7.04 (m, |
| | | | 3H), 6.05 − 5.91 (m, |
| | | | 1H), 4.98 − 4.85 (m, |
| | | | 3H), 4.61 − 4.59 (m, |
| | | | 1H), 4.49 − 4.45 (m, |
| | | | 1H), 4.38 − 4.36 (m, |
| | | | 1H), 4.15 (d, J = 6.0 |
| | | | Hz, 2H), 4.10 (s, |
| | | | 1H), 4.05 − 4.00 (m, |
| | | | 5H), 3.80 − 3.62 (m, |
| | | | 1H), 2.28 − 2.23 (m, |
| | | | 2H), 2.03 − 1.92 (m, |
| | | | 6H), 1.61 − 1.58 (m, |
| | | | 3H), 1.30 − 1.23 (m, |
| | | | 6H), 0.89 (t, J = 3.2 |
| | | | Hz, 3H) |
| | 1041 | 834.3 | ¹H NMR (400 MHz, |
| | | | CDCl₃) δ 8.84 − 8.64 |
| | | | (m, 2H), 7.99 − 7.90 |
| | | | (m, 2H), 7.80 − 7.78 |
| | | | (m, 1H), 7.64 − 7.51 |
| | | | (m, 1H), 7.35 − 7.31 |
| | | | (m, 2H), 7.24 − 7.23 |
| | | | (m, 2H), 7.19 − 7.18 |
| | | | (m, 2H), 5.99 − 5.85 |
| | | | (m, 1H), 4.97 − 4.85 |
| | | | (m, 2H), 4.60 − 4.58 |
| | | | (m, 1H), 4.49 − 4.45 |
| | | | (m, 1H), 4.34 (t, J = |
| | | | 9.6 Hz, 1H), 4.16 − |
| | | | 4.14 (m, 2H), 4.08 − |
| | | | 4.02 (m, 7H), 3.93 − |
| | | | 3.73 (m, 1H), 2.30 − |
| | | | 2.24 (m, 2H), 2.06 − |
| | | | 2.01 (m, 6H), 1.61 − |
| | | | 1.58 (m, 3H), 1.30 − |
| | | | 1.23 (m, 3H), 1.14 |
| | | | (d, J = 7.6 Hz, 3H), |
| | | | 0.89 (t, J = 7.2 Hz, |
| | | | 3H) |
| | 1042 | 821.2 | ¹H NMR (400 MHz, |
| | | | DMSO-d₆) δ 8.83 − |
| | | | 8.75 (m, 1H), 8.73 − |
| | | | 8.65 (m, 1H), 8.52 |
| | | | (d, J = 6.2 Hz, 1H), |
| | | | 8.32 − 8.19 (m, 1H), |
| | | | 8.14 − 7.99 (m, 2H), |
| | | | 7.65 − 7.56 (m, 1H), |
| | | | 7.42 − 7.29 (m, 2H), |
| | | | 7.27 − 7.07 (m, 3H), |
| | | | 6.27 − 6.17 (m, 1H), |
| | | | 6.15 − 6.05 (m, 1H), |
| | | | 4.56 − 4.50 (m, 2H), |
| | | | 4.67 (d, J = 9.1 Hz, |
| | | | 2H), 4.48 − 4.29 (m, |
| | | | 1H), 4.07 − 3.97 (m, |
| | | | 2H), 3.95 (s, 3H), |
| | | | 3.93 − 3.86 (m, 2H), |
| | | | 3.72 − 3.70 (m, 1H), |
| | | | 3.86 − 3.58 (m, 1H), |
| | | | 2.23 − 2.14 (m, 1H), |
| | | | 2.05 − 1.93 (m, 2H), |
| | | | 1.87 − 1.66 (m, 7H), |
| | | | 1.52 − 1.37 (m, 2H), |
| | | | 1.18 − 1.11 (m, 2H), |
| | | | 0.98 − 0.90 (m, 1H), |
| | | | 0.86 − 0.74 (m, 3H) |
| | 1043 | 834.4 | ¹H NMR (400 MHz, |
| | | | CD₃OD) 8.63 − 8.49 |
| | | | (m, 1H), 8.40 − 8.28 |
| | | | (m, 1H), 8.17 − 8.04 |
| | | | (m, 2H), 8.04 − 7.93 |
| | | | (m, 1H), 7.66 (d, J = |
| | | | 7.2 Hz, 1H), 7.40 − |
| | | | 7.28 (m, 2H), 7.26 − |
| | | | 7.11 (m, 3H), 6.20 − |
| | | | 5.98 (m, 1H), 4.74 |
| | | | (d, J = 10.8 Hz, 1H), |
| | | | 4.67 (t, J = 8.8 Hz, |
| | | | 1H), 4.59 − 4.49 (m, |
| | | | 2H), 4.48 − 4.36 (m, |
| | | | 1H), 4.35 − 4.26 (m, |
| | | | 1H), 4.13 − 4.02 (m, |
| | | | 2H), 4.01 − 3.95 (m, |
| | | | 1H), 3.93 − 3.76 (m, |
| | | | 2H), 3.32 (s, 6H), |
| | | | 2.36 − 2.25 (m, 1H), |
| | | | 2.17 − 2.06 (m, 2H), |
| | | | 2.05 − 1.97 (m, 2H), |
| | | | 1.97 − 1.90 (m, 2H), |
| | | | 1.89 − 1.79 (m, 3H), |
| | | | 1.62 − 1.47 (m, 2H), |
| | | | 1.27 − 1.19 (m, 2H), |
| | | | 1.09 − 1.06 (m, 1H), |
| | | | 0.93 − 0.82 (m, 3H) |
| | 1044 | 875.2 | ¹H NMR (400 MHz, |
| | | | CD₃OD) δ 8.56 − |
| | | | 8.48 (m, 1H), 8.36 − |
| | | | 8.24 (m, 1H), 8.14 − |
| | | | 8.05 (m, 2H), 8.03 − |
| | | | 7.96 (m, 1H), 7.70 − |
| | | | 7.61 (m, 1H), 7.39 − |
| | | | 7.28 (m, 3H), 7.25 − |
| | | | 7.15 (m, 3H), 6.17 − |
| | | | 5.97 (m, 1H), 4.79 − |
| | | | 4.71 (m, 2H), 4.61 − |
| | | | 4.50 (m, 2H), 4.49 − |
| | | | 4.34 (m, 1H), 4.29 − |
| | | | 4.16 (m, 1H), 4.15 − |
| | | | 4.05 (m, 2H), 4.01 − |
| | | | 3.93 (m, 2H), 3.84 |
| | | | (d, J = 4.8 Hz, 4H), |
| | | | 3.82 − 3.76 (m, 1H), |
| | | | 3.41 (s, 4H), 2.39 − |
| | | | 2.26 (m, 1H), 2.20 − |
| | | | 2.06 (m, 2H), 2.05 − |
| | | | 1.90 (m, 4H), 1.90 − |
| | | | 1.78 (m, 3H), 1.61 − |
| | | | 1.52 (m, 2H), 1.09 − |
| | | | 1.07 (m, 3H), 0.91 − |
| | | | 0.86 (m, 3H) |
| | 1045 | 875.2 | ¹H NMR (400 MHz, |
| | | | CD₃OD) δ 8.56 − |
| | | | 8.48 (m, 1H), 8.36 − |
| | | | 8.23 (m, 1H), 8.14 − |
| | | | 8.03 (m, 2H), 8.01 − |
| | | | 7.92 (m, 1H), 7.68 − |
| | | | 7.60 (m, 1H), 7.42 − |
| | | | 7.28 (m, 3H), 7.25 − |
| | | | 7.14 (m, 3H), 6.20 − |
| | | | 6.01 (m, 1H), 4.78 − |
| | | | 4.70 (m, 2H), 4.60 − |
| | | | 4.50 (m, 2H), 4.49 − |
| | | | 4.34 (m, 1H), 4.30 − |
| | | | 4.17 (m, 1H), 4.15 − |
| | | | 4.05 (m, 2H), 4.01 − |
| | | | 3.87 (m, 3H), 3.87 − |
| | | | 3.78 (m, 4H), 3.41 (s, |
| | | | 4H), 2.39 − 2.26 (m, |
| | | | 1H), 2.19 − 2.07 (m, |
| | | | 2H), 2.04 − 1.90 (m, |
| | | | 4H), 1.90 − 1.78 (m, |
| | | | 3H), 1.62 − 1.48 (m, |
| | | | 2H), 1.23 (d, J = 7.2 |
| | | | Hz, 3H), 0.93 − 0.82 |
| | | | (m, 3H) |
| | 1046 | 875.5 | ¹H NMR (400 MHz, |
| | | | CDCl₃) δ 9.03 − 8.70 |
| | | | (m, 1H), 8.49 − 8.30 |
| | | | (m, 1H), 8.07 − 7.79 |
| | | | (m, 3H), 7.64 − 7.55 |
| | | | (m, 1H), 7.29 (s, 1H), |
| | | | 7.25 (s, 1H), 7.15 − |
| | | | 7.06 (m, 3H), 7.02 − |
| | | | 6.95 (m 1H), 6.07 − |
| | | | 5.90 (m, 1H), 5.12 − |
| | | | 4.95 (m, 1H), 4.86 − |
| | | | 4.61 (m, 2H), 4.55 − |
| | | | 4.48 (m, 1H), 4.46 − |
| | | | 4.34 (m, 1H), 4.18 − |
| | | | 4.06 (m, 2H), 4.05 − |
| | | | 3.93 (m, 4H), 3.92 − |
| | | | 3.82 (m, 4H), 3.66 − |
| | | | 3.57 (m, 1H), 3.30 − |
| | | | 3.24 (m, 1H), 3.20 − |
| | | | 3.15 (m, 2H), 2.34 − |
| | | | 2.26 (m, 1H), 2.25 − |
| | | | 2.16 (m, 1H), 2.12 − |
| | | | 1.97 (m, 4H), 1.95 − |
| | | | 1.71 (m, 4H), 1.65 − |
| | | | 1.55 (m, 2H), 1.33 − |
| | | | 1.25 (m, 3H), 0.90 |
| | | | (q, J = 7.2 Hz, 3H) |
| | 1047 | 875.4 | ¹H NMR (400 MHz, |
| | | | CDCl₃) δ 9.00 − 8.68 |
| | | | (m, 1H), 8.53 − 8.27 |
| | | | (m, 1H), 8.06 − 7.78 |
| | | | (m, 3H), 7.67 − 7.49 |
| | | | (m, 1H), 7.40 − 7.31 |
| | | | (m, 2H), 7.28 ( s, |
| | | | 1H), 7.25 ( s, 1H), |
| | | | 7.19 (q, J = 7.0 Hz, |
| | | | 1H), 7.10 − 7.01 (m, |
| | | | 1H), 6.03 − 5.85 (m, |
| | | | 1H), 5.11 − 4.93 (m, |
| | | | 1H), 4.84 − 4.64 (m, |
| | | | 2H), 4.55 − 4.49 (m, |
| | | | 1H), 4.42 − 4.34 (m, |
| | | | 1H), 4.16 − 4.10 (m, |
| | | | 1H), 4.09 − 4.02 (m, |
| | | | 3H), 4.01 − 3.95 (m, |
| | | | 1H), 3.92 − 3.85 (m, |
| | | | 4H), 3.77 − 3.77 (m, |
| | | | 1H), 3.33 − 3.06 (m, |
| | | | 4H), 2.34 − 2.24 (m, |
| | | | 1H), 2.23 − 2.13 (m, |
| | | | 1H), 2.12 − 1.96 (m, |
| | | | 4H), 1.94 − 1.69 (m, |
| | | | 4H), 1.68 − 1.55 (m, |
| | | | 2H), 1.15 (dd, J = |
| | | | 7.2, 14.8 Hz, 3H), |
| | | | 0.90 (dt, J = 4.0, 7.2 |
| | | | Hz, 3H) |
| | 1048 | 818.4 | ¹H NMR (400 MHz, |
| | | | CDCl₃) δ 9.26 − 8.91 |
| | | | (m, 1H), 8.71 − 8.59 |
| | | | (m, 1H), 8.22 (d, J = |
| | | | 7.6 Hz, 1H), 8.02 (s, |
| | | | 1H), 7.96 (s, 1H), |
| | | | 7.87 − 7.80 (m, 1H), |
| | | | 7.67 − 7.51 (m, 3H), |
| | | | 7.39 − 7.30 (m, 2H), |
| | | | 7.24 (s, 1H), 7.22 − |
| | | | 7.16 (m, 1H), 6.05 − |
| | | | 5.88 (m, 1H), 5.11 − |
| | | | 5.08 (m, 1H), 4.85 − |
| | | | 4.71 (m, 2H), 4.56 − |
| | | | 4.42 (m, 2H), 4.27 − |
| | | | 4.02 (m, 2H), 4.00 − |
| | | | 3.87 (m, 3H), 3.85 − |
| | | | 3.64 (m, 2H), 2.51 (s, |
| | | | 3H), 2.36 − 2.17 (m, |
| | | | 2H), 2.14 − 1.99 (m, |
| | | | 4H), 1.80 (t, J = 12.4 |
| | | | Hz, 1H), 1.73 − 1.60 |
| | | | (m, 1H), 1.58 − 1.45 |
| | | | (m, 3H), 1.27 (d, J = |
| | | | 6.8 Hz, 3H), 1.05 − |
| | | | 0.99 (m, 3H), 0.86 (t, |
| | | | J = 7.6 Hz, 3H) |
| | 1049 | 804.2 | ¹H NMR (400 MHz, |
| | | | CDCl₃) δ 9.28 − 8.86 |
| | | | (m, 1H), 8.74 − 8.54 |
| | | | (m, 1H), 8.27 − 8.01 |
| | | | (m, 1H), 7.96 (s, 1H), |
| | | | 7.88 − 7.79 (m, 1H), |
| | | | 7.67 − 7.52 (m, 2H), |
| | | | 7.40 − 7.29 (m, 2H), |
| | | | 7.27 − 7.12 (m, 3H), |
| | | | 6.04 − 5.89 (m, 1H), |
| | | | 5.15 − 5.04 (m, 1H), |
| | | | 4.85 − 4.66 (m, 2H), |
| | | | 4.57 − 4.51 (m, 1H), |
| | | | 4.50 − 4.39 (m, 1H), |
| | | | 4.12 − 3.83 (m, 5H), |
| | | | 3.79 − 3.63 (m, 1H), |
| | | | 2.50 (s, 3H), 2.36 − |
| | | | 2.15 (m, 2H), 2.12 − |
| | | | 1.70 (m, 8H), 1.53 |
| | | | (q, J = 7.2 Hz, 2H), |
| | | | 1.33 − 1.22 (m, 3H), |
| | | | 0.92 − 0.79 (m, 3H) |
| | 1050 | 868.3 | ¹H NMR (400 MHz, |
| | | | CD₃OD) δ 8.72 − |
| | | | 8.57 (m, 2H), 8.14 − |
| | | | 7.97 (m, 2H), 7.95 − |
| | | | 7.86 (m, 1H), 7.66 − |
| | | | 7.53 (m, 2H), 7.37 − |
| | | | 7.24 (m, 7H), 7.21 − |
| | | | 7.12 (m, 3H), 6.12 − |
| | | | 5.92 (m, 1H), 5.07 − |
| | | | 4.99 (m, 1H), 4.97 − |
| | | | 4.90 (m, 2H), 4.77 − |
| | | | 4.71 (m, 1H), 4.66 |
| | | | (d, J = 8.0 Hz, 1H), |
| | | | 4.55 (t, J = 4.8 Hz, |
| | | | 1H), 4.48 − 4.38 (m, |
| | | | 1H), 4.37 − 4.30 (m, |
| | | | 1H), 4.18 (s, 3H), |
| | | | 4.17 − 4.13 (m, 1H), |
| | | | 4.09 (d, J = 5.6 Hz, |
| | | | 1H), 3.89 (d, J = 7.2 |
| | | | Hz, 1H), 2.38 − 2.25 |
| | | | (m, 1H), 2.20 − 2.05 |
| | | | (m, 2H), 2.04 − 1.90 |
| | | | (m, 4H), 1.89 − 1.77 |
| | | | (m, 3H), 1.22 (d, J = |
| | | | 7.2 Hz, 3H) |
| | 1051 | 868.3 | ¹H NMR (400 MHz, |
| | | | CDCl₃) δ 8.78 − 8.65 |
| | | | (m, 1H), 8.07 − 7.89 |
| | | | (m, 2H), 7.87 − 7.77 |
| | | | (m, 1H), 7.55 − 7.45 |
| | | | (m, 1H), 7.38 − 7.29 |
| | | | (m, 6H), 7.28 − 7.27 |
| | | | (m, 1H), 7.27 − 7.25 |
| | | | (m, 2H), 7.25 − 7.22 |
| | | | (m, 1H), 7.19 (d, J = |
| | | | 6.8 Hz, 1H), 7.16 (d, |
| | | | J = 7.2 Hz, 1H), 6.04 − |
| | | | 5.83 (m, 1H), 5.13 |
| | | | (s, 2H), 5.01 (t, J = |
| | | | 9.6 Hz, 1H), 4.80 − |
| | | | 4.67 (m, 1H), 4.67 − |
| | | | 4.59 (m, 1H), 4.54 (s, |
| | | | 1H), 4.42 − 4.34 (m, |
| | | | 1H), 4.26 − 4.17 (m, |
| | | | 1H), 4.16 − 4.10 (m, |
| | | | 3H), 4.08 − 4.03 (m, |
| | | | 1H), 4.00 − 3.92 (m, |
| | | | 1H), 3.88 − 3.75 (m, |
| | | | 1H), 2.32 − 2.25 (m, |
| | | | 1H), 2.23 − 2.10 (m, |
| | | | 2H), 2.03 (dd, J = |
| | | | 6.8, 12.0 Hz, 3H), |
| | | | 1.96 − 1.81 (m, 3H), |
| | | | 1.79 − 1.67 (m, 1H), |
| | | | 1.15 (t, J = 7.2 Hz, |
| | | | 3H |
| | 1052 | 820.7 | ¹H NMR (400 MHz, |
| | | | CD₃OD) δ 8.61 (m, |
| | | | 2H), 8.12 − 8.02 (m, |
| | | | 2H), 7.95 (m, 1H), |
| | | | 7.56 (m, 2H), 7.36 − |
| | | | 7.28 (m, 2H), 7.16 |
| | | | (m, 3H), 6.25 − 5.98 |
| | | | (m, 1H), 4.95 − 4.87 |
| | | | (m, 1H), 4.77 − 4.70 |
| | | | (m, 1H), 4.64 (s, 1H), |
| | | | 4.58 − 4.52 (m, 1H), |
| | | | 4.45 − 4.35 (m, 1H), |
| | | | 4.18 (s, 6H), 4.10 − |
| | | | 4.03 (m, 1H), 3.86 − |
| | | | 3.75 (m, 1H), 2.36 − |
| | | | 2.25 (m, 1H), 2.16 − |
| | | | 2.05 (m, 2H), 2.04 − |
| | | | 1.94 (m, 3H), 1.94 − |
| | | | 1.87 (m, 2H), 1.84 |
| | | | (m, 2H), 1.23 − 1.15 |
| | | | (m, 6H), 1.12 − 1.06 |
| | | | (m, 3H) |
| | 1053 | 820.4 | ¹H NMR (400 MHz, |
| | | | CD₃OD) 8.73 − 8.52 |
| | | | (m, 2H), 8.13 − 8.02 |
| | | | (m, 2H), 8.01 − 7.91 |
| | | | (m, 1H), 7.68 − 7.54 |
| | | | (m, 2H), 7.42 − 7.27 |
| | | | (m, 2H), 7.18 (d, J = |
| | | | 7.6 Hz, 3H), 6.22 − |
| | | | 5.98 (m, 1H), 4.78 − |
| | | | 4.71 (m, 1H), 4.68 − |
| | | | 4.62 (m, 1H), 4.56 − |
| | | | 4.53 (m, 1H), 4.50 − |
| | | | 4.41 (m, 1H), 4.40 − |
| | | | 4.25 (m, 1H), 4.19 (s, |
| | | | 5H), 4.08 (d, J = 3.6 |
| | | | Hz, 1H), 4.01 − 3.84 |
| | | | (m, 3H), 2.39 − 2.25 |
| | | | (m, 1H), 2.21 − 2.06 |
| | | | (m, 2H), 2.02 − 1.98 |
| | | | (m, 1H), 1.96 − 1.91 |
| | | | (m, 2H), 1.90 − 1.79 |
| | | | (m, 3H), 1.62 − 1.46 |
| | | | (m, 2H), 1.38 − 1.36 |
| | | | (m, 1H), 1.24 (d, J = |
| | | | 7.2 Hz, 3H), 0.94 − |
| | | | 0.81 (m, 3H) |
| | 1054 | 868.2 | ¹H NMR (400 MHz, |
| | | | CDCl₃) δ 9.02 − 8.67 |
| | | | (m, 2H), 8.13 − 7.93 |
| | | | (m, 2H), 7.89 − 7.79 |
| | | | (m, 1H), 7.60 − 7.52 |
| | | | (m, 1H), 7.41 − 7.27 |
| | | | (m, 6H), 7.22 (t, J = |
| | | | 7.2 Hz, 2H), 7.17 − |
| | | | 7.05 (m, 3H), 6.02 − |
| | | | 5.85 (m, 1H), 5.19 − |
| | | | 5.05 (m, 2H), 5.04 − |
| | | | 4.90 (m, 1H), 4.83 − |
| | | | 4.70 (m, 1H), 4.68 − |
| | | | 4.58 (m, 1H), 4.54 (t, |
| | | | J = 5.2 Hz, 1H), 4.43 − |
| | | | 4.33 (m, 1H), 4.25 − |
| | | | 4.18 (m, 1H), 4.11 |
| | | | (d, J = 3.0 Hz, 3H), |
| | | | 4.08 − 4.02 (m, 1H), |
| | | | 3.96 (s, 1H), 3.76 − |
| | | | 3.64 (m, 1H), 2.34 − |
| | | | 2.22 (m, 1H), 2.20 − |
| | | | 1.97 (m, 5H), 1.95 − |
| | | | 1.66 (m, 4H), 1.31 |
| | | | (d, J = 7.2 Hz, 3H) |
| | 1055 | 868.5 | ¹H NMR (400 MHz, |
| | | | CDCl₃) δ 8.88 − 8.38 |
| | | | (m, 2H), 8.06 − 7.89 |
| | | | (m, 2H), 7.84 − 7.61 |
| | | | (m, 2H), 7.51 (t, J = |
| | | | 8.0 Hz, 1H), 7.43 − |
| | | | 7.27 (m, 5H), 7.24 |
| | | | (d, J = 7.6 Hz, 4H), |
| | | | 7.02 − 6.98 (m, 1H), |
| | | | 6.07 − 5.84 (m, 1H), |
| | | | 5.11 − 4.97 (m, 2H), |
| | | | 4.96 − 4.82 (m, 1H), |
| | | | 4.79 − 4.64 (m, 1H), |
| | | | 4.62 − 4.47 (m, 2H), |
| | | | 4.42 − 4.27 (m, 1H), |
| | | | 4.22 − 4.11 (m, 1H), |
| | | | 4.05 − 3.97 (m, 3H), |
| | | | 3.97 − 3.83 (m, 2H), |
| | | | 3.80 − 3.63 (m, 1H), |
| | | | 2.33 − 2.21 (m, 1H), |
| | | | 2.21 − 1.98 (m, 5H), |
| | | | 1.98 − 1.60 (m, 4H), |
| | | | 1.28 − 1.26 (m, 3H) |
| | 1056 | 848.3 | ¹H NMR (400 MHz, |
| | | | CD₃OD) δ 8.71 − |
| | | | 8.57 (m, 2H), 8.13 − |
| | | | 8.05 (m, 2H), 8.03 − |
| | | | 7.97 (m, 1H), 7.68 − |
| | | | 7.58 (m, 2H), 7.35 − |
| | | | 7.28 (m, 2H), 7.24 − |
| | | | 7.13 (m, 3H), 6.15 − |
| | | | 6.00 (m, 1H), 4.78 − |
| | | | 4.71 (m, 2H), 4.66 |
| | | | (d, J = 6.4 Hz, 1H), |
| | | | 4.55 (dt, J = 4.8, 8.0 |
| | | | Hz, 1H), 4.46 − 4.34 |
| | | | (m, 1H), 4.20 (d, J = |
| | | | 2.8 Hz, 3H), 4.19 − |
| | | | 4.14 (m, 2H), 4.13 − |
| | | | 4.04 (m, 1H), 3.90 − |
| | | | 3.81 (m, 1H), 3.80 − |
| | | | 3.70 (m, 2H), 2.37 − |
| | | | 2.25 (m, 1H), 2.18 − |
| | | | 2.06 (m, 2H), 2.05 − |
| | | | 1.91 (m, 4H), 1.90 − |
| | | | 1.79 (m, 3H), 1.11 |
| | | | (dd, J = 4.0, 7.2 Hz, |
| | | | 3H), 0.89 (s, 9H) |
| | 1057 | 848.4 | ¹H NMR (400 MHz, |
| | | | CD₃OD) δ 8.71 − |
| | | | 8.58 (m, 2H), 8.15 − |
| | | | 8.04 (m, 2H), 8.02 − |
| | | | 7.92 (m, 1H), 7.67 − |
| | | | 7.57 (m, 2H), 7.39 − |
| | | | 7.31 (m, 2H), 7.26 − |
| | | | 7.13 (m, 3H), 6.19 − |
| | | | 6.03 (m, 1H), 4.90 (s, |
| | | | 1H), 4.78 − 4.72 (m, |
| | | | 1H), 4.65 (d, J = 6.4 |
| | | | Hz, 1H), 4.56 (dt, J = |
| | | | 4.8, 8.4 Hz, 1H), 4.46 − |
| | | | 4.33 (m, 1H), 4.23 − |
| | | | 4.15 (m, 5H), 4.08 |
| | | | (d, J = 4.8 Hz, 1H), |
| | | | 3.93 − 3.85 (m, 1H), |
| | | | 3.80 − 3.73 (m, 1H), |
| | | | 3.62 − 3.54 (m, 1H), |
| | | | 2.37 − 2.26 (m, 1H), |
| | | | 2.19 − 2.05 (m, 2H), |
| | | | 2.04 − 1.90 (m, 4H), |
| | | | 1.89 − 1.78 (m, 3H), |
| | | | 1.25 (d, J = 7.2 Hz, |
| | | | 3H), 0.86 (d, J = 2.4 |
| | | | Hz, 9H) |
| | 1058 | 834.4 | ¹H NMR (400 MHz, |
| | | | CDCl₃) δ 8.95 − 8.67 |
| | | | (m, 2H), 8.02 − 7.80 |
| | | | (m, 3H), 7.68 − 7.56 |
| | | | (m, 1H), 7.30 − 7.25 |
| | | | (m, 3H), 7.25 − 7.20 |
| | | | (m, 1H), 7.19 − 7.05 |
| | | | (m, 3H), 6.10 − 5.88 |
| | | | (m, 1H), 5.02 − 4.89 |
| | | | (m, 1H), 4.79 − 4.68 |
| | | | (m, 1H), 4.64 − 4.58 |
| | | | (m, 1H), 4.55 (t, J = |
| | | | 5.6 Hz, 1H), 4.41 − |
| | | | 4.34 (m, 1H), 4.23 − |
| | | | 4.15 (m, 1H), 4.14 − |
| | | | 4.09 (m, 3H), 4.07 − |
| | | | 3.93 (m, 4H), 3.73 − |
| | | | 3.55 (m, 1H), 2.35 − |
| | | | 2.24 (m, 1H), 2.19 − |
| | | | 1.92 (m, 5H), 1.84 − |
| | | | 1.71 (m, 1H), 1.71 − |
| | | | 1.42 (m, 4H), 1.36 − |
| | | | 1.23 (m, 3H), 1.07 − |
| | | | 0.98 (m, 3H), 0.96 − |
| | | | 0.83 (m, 3H) |
| | 1059 | 834.3 | ¹H NMR (400 MHz, |
| | | | CDCl₃) δ 8.91 − 8.67 |
| | | | (m, 2H), 8.02 − 7.81 |
| | | | (m, 3H), 7.68 − 7.51 |
| | | | (m, 1H), 7.40 − 7.31 |
| | | | (m, 2H), 7.29 − 7.25 |
| | | | (m, 3H), 7.23 − 7.15 |
| | | | (m, 2H), 6.03 − 5.86 |
| | | | (m, 1H), 5.02 − 4.89 |
| | | | (m, 1H), 4.79 − 4.69 |
| | | | (m, 1H), 4.65 − 4.58 |
| | | | (m, 1H), 4.55 (t, J = |
| | | | 5.6 Hz, 1H), 4.41 − |
| | | | 4.34 (m, 1H), 4.23 − |
| | | | 4.16 (m, 1H), 4.14 − |
| | | | 4.03 (m, 6H), 4.00 − |
| | | | 3.92 (m, 1H), 3.84 − |
| | | | 3.65 (m, 1H), 2.35 − |
| | | | 2.23 (m, 1H), 2.18 − |
| | | | 1.91 (m, 5H), 1.84 − |
| | | | 1.73 (m, 1H), 1.71 − |
| | | | 1.43 (m, 4H), 1.20 − |
| | | | 1.09 (m, 3H), 1.06 − |
| | | | 0.98 (m, 3H), 0.94 − |
| | | | 0.84 (m, 3H) |
| | 1060 | 824.5 | ¹H NMR (400 MHz, |
| | | | CD₃OD) δ 8.13 − |
| | | | 8.04 (m, 2H), 8.02 − |
| | | | 7.93 (m, 2H), 7.91 − |
| | | | 7.77 (m, 1H), 7.68 − |
| | | | 7.61 (m, 1H), 7.37 − |
| | | | 7.29 (m, 2H), 7.22 − |
| | | | 7.13 (m, 3H), 6.19 − |
| | | | 6.01 (m, 1H), 4.79 − |
| | | | 4.72 (m, 2H), 4.66 − |
| | | | 4.52 (m, 2H), 4.51 − |
| | | | 4.41 (m, 1H), 4.39 − |
| | | | 4.31 (m, 1H), 4.10 − |
| | | | 3.98 (m, 2H), 3.95 − |
| | | | 3.87 (m, 1H), 3.86 − |
| | | | 3.78 (m, 2H), 2.35 − |
| | | | 2.25 (m, 1H), 2.18 − |
| | | | 2.05 (m, 2H), 2.04 − |
| | | | 1.87 (m, 5H), 1.87 − |
| | | | 1.77 (m, 2H), 1.55 − |
| | | | 1.44 (m, 2H), 1.21 |
| | | | (d, J = 7.2 Hz, 3H), |
| | | | 0.90 − 0.81 (m, 3H) |
| | 1061 | 870.5 | ¹H NMR (400 MHz, |
| | | | CD₃OD) δ 8.46 − |
| | | | 8.37 (m, 1H), 8.19 − |
| | | | 8.07 (m, 3H), 8.05 − |
| | | | 7.96 (m, 1H), 7.71 − |
| | | | 7.63 (m, 1H), 7.40 − |
| | | | 7.30 (m, 2H), 7.30 − |
| | | | 7.12 (m, 3H), 6.70 − |
| | | | 6.60 (m, 1H), 6.22 − |
| | | | 6.00 (m, 1H), 4.75 (t, |
| | | | J = 12.0 Hz, 3H), |
| | | | 4.70 − 4.64 (m, 2H), |
| | | | 4.64 − 4.59 (m, 1H), |
| | | | 4.59 − 4.54 (m, 1H), |
| | | | 4.54 − 4.18 (m, 2H), |
| | | | 4.17 − 4.06 (m, 3H), |
| | | | 4.02 − 3.92 (m, 3H), |
| | | | 3.91 − 3.90 (m, 1H), |
| | | | 2.39 − 2.28 (m, 1H), |
| | | | 2.21 − 2.08 (m, 2H), |
| | | | 2.08 − 1.93 (m, 4H), |
| | | | 1.92 − 1.82 (m, 3H), |
| | | | 1.64 − 1.51 (m, 2H), |
| | | | 1.25 (d, J = 7.2 Hz, |
| | | | 1H), 1.13 − 1.07 (m, |
| | | | 2H), 0.94 − 0.85 (m, |
| | | | 3H) |
| | 1062 | 875.4 | IH NMR (400 MHz, |
| | | | CD₃OD) δ 8.31 (d, J = |
| | | | 8.8 Hz, 1H), 8.15 − |
| | | | 7.92 (m, 4H), 7.69 − |
| | | | 7.61 (m, 1H), 7.38 − |
| | | | 7.28 (m, 2H), 7.24 − |
| | | | 7.12 (m, 3H), 6.58 |
| | | | (dd, J = 7.2, 12.0 Hz, |
| | | | 1H), 6.19 − 5.99 (m, |
| | | | 1H), 4.87 (s, IH), |
| | | | 4.74 (d, J = 11.2 Hz, |
| | | | IH), 4.66 − 4.51 (m, |
| | | | 4H), 4.44 − 4.30 (m, |
| | | | 3H), 4.30 − 4.24 (m, |
| | | | 1H), 4.22 − 4.15 (m, |
| | | | 1H), 4.09 (d, J = 6.8 |
| | | | Hz, 2H), 3.98 (t, J = |
| | | | 6.8 Hz, 1H), 3.94 − |
| | | | 3.79 (m, 2H), 3.37 (s, |
| | | | 3H), 2.38 − 2.24 (m, |
| | | | 1H), 2.21 − 2.06 (m, |
| | | | 2H), 2.05 − 1.80 (m, |
| | | | 7H), 1.61 − 1.46 (m, |
| | | | 2H), 1.27 − 1.19 (m, |
| | | | 3H), 0.94 − 0.80 (m, |
| | | | 3H) |
| | 1063 | 875.4 | ¹H NMR (400 MHz, |
| | | | CD₃OD) δ 8.30 (d, |
| | | | J = 8.4 Hz, 1H), 8.15 − |
| | | | 7.99 (m, 4H), 7.70 − |
| | | | 7.60 (m, 1H), 7.42 − |
| | | | 7.29 (m, 2H), 7.27 − |
| | | | 7.12 (m, 3H), 6.58 |
| | | | (dd, J = 6.8, 11.6 Hz, |
| | | | 1H), 6.20 − 6.02 (m, |
| | | | 1H), 4.87 − 4.85 (m, |
| | | | 1H), 4.75 (d, J = 11.2 |
| | | | Hz, 1H), 4.66 − 4.51 |
| | | | (m, 4H), 4.44 − 4.30 |
| | | | (m, 3H), 4.28 (d, J = |
| | | | 1.2 Hz, 1H), 4.23 − |
| | | | 4.15 (m, 1H), 4.09 |
| | | | (d, J = 6.4 Hz, 2H), |
| | | | 4.01 − 3.76 (m, 3H), |
| | | | 3.35 (d, J = 19.2 Hz, |
| | | | 3H), 2.38 − 2.25 (m, |
| | | | 1H), 2.21 − 2.07 (m, |
| | | | 2H), 2.05 − 1.90 (m, |
| | | | 4H), 1.89 − 1.79 (m, |
| | | | 3H), 1.64 − 1.47 (m, |
| | | | 2H), 1.26 − 1.05 (m, |
| | | | 3H), 0.95 − 0.86 (m, |
| | | | 3H) |
| | 1064 | 875.5 | ¹H NMR (400 MHz, |
| | | | CDCl₃) δ 8.98 − 8.67 |
| | | | (m, 1H), 8.50 − 8.28 |
| | | | (m, 2H), 8.11 − 7.98 |
| | | | (m, 1H), 7.98 − 7.80 |
| | | | (m, 2H), 7.67 − 7.57 |
| | | | (m, 1H), 7.56 − 7.27 |
| | | | (m, 2H), 7.26 − 7.08 |
| | | | (m, 3H), 7.08 − 6.98 |
| | | | (m, 1H), 6.06 − 5.88 |
| | | | (m, 1H), 5.11 − 4.93 |
| | | | (m, 1H), 4.86 − 4.64 |
| | | | (m, 2H), 4.54 − 4.36 |
| | | | (m, 2H), 4.15 − 3.92 |
| | | | (m, 6H), 3.91 − 3.86 |
| | | | (m, 4H), 3.79 − 3.57 |
| | | | (m, 1H), 3.35 − 3.14 |
| | | | (m, 4H), 2.35 − 2.25 |
| | | | (m, 1H), 2.24 − 2.14 |
| | | | (m, 1H), 2.14 − 1.97 |
| | | | (m, 4H), 1.96 − 1.68 |
| | | | (m, 4H), 1.66 − 1.55 |
| | | | (m, 2H), 1.35 − 1.11 |
| | | | (m, 3H), 0.95 − 0.86 |
| | | | (m, 3H) |
| | 1065 | 875.6 | ¹H NMR (400 MHz, |
| | | | CD₃OD) δ 8.56 − |
| | | | 8.47 (m, 1H), 8.28 |
| | | | (d, J = 6.0 Hz, 1H), |
| | | | 8.13 − 8.09 (m, 1H), |
| | | | 8.07 (d, J = 3.2 Hz, |
| | | | 1H), 8.03 − 7.93 (m, |
| | | | 1H), 7.69 − 7.60 (m, |
| | | | 1H), 7.39 − 7.30 (m, |
| | | | 2H), 7.29 − 7.14 (m, |
| | | | 4H), 6.21 − 5.99 (m, |
| | | | 1H), 4.78 − 4.71 (m, |
| | | | 2H), 4.65 − 4.51 (m, |
| | | | 2H), 4.49 − 4.35 (m, |
| | | | 1H), 4.29 − 4.20 (m, |
| | | | 1H), 4.16 − 4.05 (m, |
| | | | 2H), 4.02 − 3.87 (m, |
| | | | 3H), 3.87 − 3.79 (m, |
| | | | 4H), 3.23 (s, 4H), |
| | | | 2.39 − 2.26 (m, 1H), |
| | | | 2.21 − 2.04 (m, 3H), |
| | | | 2.04 − 2.00 (m, 1H), |
| | | | 1.99 − 1.96 (m, 1H), |
| | | | 1.95 − 1.88 (m, 2H), |
| | | | 1.87 − 1.79 (m, 2H), |
| | | | 1.61 − 1.49 (m, 2H), |
| | | | 1.26 − 1.04 (m, 3H), |
| | | | 0.92 − 0.83 (m, 3H) |
| | 1066 | 876.3 | ¹H NMR (400 MHz, |
| | | | CD₃OD) δ 8.65 − |
| | | | 8.53 (m, 1H), 8.39 |
| | | | (d, J = 6.8 Hz, 1H), |
| | | | 8.13 − 8.03 (m, 2H), |
| | | | 8.01 − 7.92 (m, 1H), |
| | | | 7.69 − 7.58 (m, 1H), |
| | | | 7.39 − 7.29 (m, 2H), |
| | | | 7.26 − 7.12 (m, 3H), |
| | | | 6.23 − 5.97 (m, 1H), |
| | | | 4.78 − 4.70 (m, 1H), |
| | | | 4.59 − 4.44 (m, 2H), |
| | | | 4.43 − 4.28 (m, 1H), |
| | | | 4.20 − 4.12 (m, 1H), |
| | | | 4.12 − 4.02 (m, 2H), |
| | | | 4.01 − 3.95 (m, 1H), |
| | | | 3.95 − 3.87 (m, 1H), |
| | | | 3.85 (d, J = 7.2 Hz, |
| | | | 1H), 3.83 − 3.76 (m, |
| | | | 2H), 3.76 (s, 4H), |
| | | | 3.73 − 3.61 (m, 3H), |
| | | | 2.37 − 2.24 (m, 1H), |
| | | | 2.20 − 2.05 (m, 2H), |
| | | | 2.04 − 1.89 (m, 4H), |
| | | | 1.88 − 1.73 (m, 3H), |
| | | | 1.64 − 1.44 (m, 2H), |
| | | | 1.27 − 1.17 (m, 3H), |
| | | | 0.94 − 0.79 (m, 3H) |
| | 1067 | 876.5 | ¹H NMR (400 MHz, |
| | | | CD₃OD) δ 8.69 − |
| | | | 8.54 (m, 1H), 8.38 |
| | | | (d, J = 9.2 Hz, 1H), |
| | | | 8.15 − 8.04 (m, 2H), |
| | | | 8.03 − 7.93 (m, 1H), |
| | | | 7.70 − 7.60 (m, 1H), |
| | | | 7.41 − 7.29 (m, 2H), |
| | | | 7.26 − 7.14 (m, 3H), |
| | | | 6.19 − 5.99 (m, 1H), |
| | | | 4.78 − 4.65 (m, 2H), |
| | | | 4.59 − 4.44 (m, 2H), |
| | | | 4.42 − 4.30 (m, 1H), |
| | | | 4.25 − 4.13 (m, 1H), |
| | | | 4.12 − 4.01 (m, 2H), |
| | | | 4.00 − 3.93 (m, 2H), |
| | | | 3.93 − 3.87 (m, 1H), |
| | | | 3.87 − 3.80 (m, 4H), |
| | | | 3.76 (s, 4H), 2.37 − |
| | | | 2.25 (m, 1H), 2.19 − |
| | | | 2.06 (m, 2H), 2.03 − |
| | | | 1.89 (m, 4H), 1.88 − |
| | | | 1.77 (m, 3H), 1.60 − |
| | | | 1.50 (m, 2H), 1.23 |
| | | | (d, J = 7.1 Hz, 1H), |
| | | | 1.08 (d, J = 7.2 Hz, |
| | | | 2H), 0.91 − 0.84 (m, |
| | | | 3H) |
| | 1068 | 834.6 | ¹H NMR (400 MHz, |
| | | | CDCl₃) δ 8.78 − 8.59 |
| | | | (m, 1H), 8.52 − 8.20 |
| | | | (m, 1H), 8.04 − 7.90 |
| | | | (m, 1H), 7.88 − 7.79 |
| | | | (m, 1H), 7.63 − 7.54 |
| | | | (m, 1H), 7.41 − 7.31 |
| | | | (m, 1H), 7.29 − 7.27 |
| | | | (m, 1H), 7.27 − 7.25 |
| | | | (m, 1H), 7.25 − 7.20 |
| | | | (m, 1H), 7.19 − 7.05 |
| | | | (m, 2H), 6.09 − 5.84 |
| | | | (m, 1H), 5.06 − 4.98 |
| | | | (m, 1H), 4.84 (d, J = |
| | | | 6.4 Hz, 1H), 4.73 − |
| | | | 4.55 (m, 2H), 4.53 − |
| | | | 4.46 (m, 1H), 4.41 − |
| | | | 4.32 (m, 1H), 4.17 − |
| | | | 4.10 (m, 1H), 4.06 − |
| | | | 4.02 (m, 1H), 4.00 − |
| | | | 3.92 (m, 2H), 3.88 − |
| | | | 3.76 (m, 1H), 3.75 − |
| | | | 3.63 (m, 1H), 3.36 − |
| | | | 3.18 (m, 6H), 2.34 − |
| | | | 2.13 (m, 2H), 2.11 − |
| | | | 1.89 (m, 5H), 1.73 − |
| | | | 1.57 (m, 2H), 1.56 − |
| | | | 1.45 (m, 2H), 1.42 |
| | | | (d, J = 6.8 Hz, 1H), |
| | | | 1.31 − 1.25 (m, 3H), |
| | | | 0.96 − 0.81 (m, 3H) |
| | 1069 | 834.5 | ¹H NMR (400 MHz, |
| | | | CD₃OD) δ 8.48 − |
| | | | 8.30 (m, 2H), 8.14 − |
| | | | 8.07 (m, 2H), 8.05 − |
| | | | 7.95 (m, 1H), 7.71 − |
| | | | 7.62 (m, 1H), 7.39 − |
| | | | 7.30 (m, 2H), 7.26 − |
| | | | 7.15 (m, 3H), 6.20 − |
| | | | 6.01 (m, 1H), 4.81 − |
| | | | 4.66 (m, 2H), 4.65 − |
| | | | 4.46 (m, 2H), 4.45 − |
| | | | 4.32 (m, 1H), 4.27 − |
| | | | 4.15 (m, 1H), 4.03 (s, |
| | | | 2H), 4.02 − 3.97 (m, |
| | | | 1H), 3.96 − 3.80 (m, |
| | | | 2H), 3.04 (d, J = 2.0 |
| | | | Hz, 6H), 2.38 − 2.25 |
| | | | (m, 1H), 2.18 − 2.06 |
| | | | (m, 2H), 2.05 − 1.92 |
| | | | (m, 4H), 1.90 − 1.78 |
| | | | (m, 3H), 1.64 − 1.47 |
| | | | (m, 2H), 1.27 − 1.17 |
| | | | (m, 3H), 0.93 − 0.83 |
| | | | (m, 3H) |
| | 1070 | 816.6 | ¹H NMR (400 MHz, |
| | | | CD₃OD) δ 8.49 − |
| | | | 8.24 (m, 2H), 8.03 |
| | | | (d, J = 9.2 Hz, 1H), |
| | | | 7.94 − 7.83 (m, 2H), |
| | | | 7.53 − 7.44 (m, 1H), |
| | | | 7.36 − 7.26 (m, 2H), |
| | | | 7.16 (dd, J = 8.0, 15.2 |
| | | | Hz, 3H), 4.86 − 4.63 |
| | | | (m, 2H), 4.62 − 4.43 |
| | | | (m, 2H), 4.42 − 4.28 |
| | | | (m, 1H), 4.25 − 4.14 |
| | | | (m, 1H), 4.12 − 3.99 |
| | | | (m, 2H), 3.98 − 3.78 |
| | | | (m, 3H), 3.62 − 3.43 |
| | | | (m, 2H), 3.02 (d, J = |
| | | | 1.6 Hz, 6H), 2.38 − |
| | | | 2.20 (m, 1H), 2.18 − |
| | | | 1.99 (m, 3H), 1.98 − |
| | | | 1.76 (m, 6H), 1.62 − |
| | | | 1.46 (m, 2H), 1.21 − |
| | | | 1.02 (m, 3H), 1.00 − |
| | | | 0.86 (m, 3H) |
| | 1071 | 890.3 | ¹H NMR (400 MHz, |
| | | | CD₃OD) δ 8.52 (d, J = |
| | | | 18 Hz, 1H), 8.29 (s, |
| | | | 1H), 8.14 − 8.04 (m, |
| | | | 2H), 8.02 − 7.94 (m, |
| | | | 1H), 7.69 − 7.60 (m, |
| | | | 1H), 7.37 − 7.27 (m, |
| | | | 2H), 7.24 − 7.13 (m, |
| | | | 3H), 6.24 − 5.97 (m, |
| | | | 1H), 4.90-4.89 (m, |
| | | | 1H), 4.83 (s, 1H), |
| | | | 4.78 − 4.71 (m, 1H), |
| | | | 4.62 − 4.50 (m, 3H), |
| | | | 4.45 − 4.37 (m, 1H), |
| | | | 4.33 (s, 1H), 4.13 − |
| | | | 4.02 (m, 3H), 4.01 − |
| | | | 3.95 (m, 2H), 3.94 − |
| | | | 3.79 (m, 2H), 3.62 − |
| | | | 3.56 (m, 1H), 3.38 |
| | | | (d, J = 5.6 Hz, 3H), |
| | | | 2.63 − 2.50 (m, 1H), |
| | | | 2.49 − 2.36 (m, 1H), |
| | | | 2.36 − 2.25 (m, 1H), |
| | | | 2.20 − 2.06 (m, 2H), |
| | | | 2.04 (s, 3H), 1.92 − |
| | | | 1.76 (m, 4H), 1.62 − |
| | | | 1.48 (m, 2H), 1.29 (s, |
| | | | 3H), 0.93 − 0.82 (m, |
| | | | 3H) |
| | 1072 | 872.7 | ¹H NMR (400 MHz, |
| | | | CD₃OD) δ 8.58 − |
| | | | 8.50 (m, 1H), 8.30 (s, |
| | | | 1H), 8.07 − 8.01 (m, |
| | | | 1H), 7.95 − 7.86 (m, |
| | | | 2H), 7.53 − 7.46 (m, |
| | | | 1H), 7.37 − 7.29 (m, |
| | | | 2H), 7.21 − 7.13 (m, |
| | | | 3H), 4.94 (d, J = 3.2 |
| | | | Hz, 1H), 4.74 (d, J = |
| | | | 11.2 Hz, 1H), 4.64 − |
| | | | 4.57 (m, 2H), 4.56 − |
| | | | 4.51 (m, 1H), 4.51 − |
| | | | 4.25 (m, 3H), 4.14 − |
| | | | 3.99 (m, 4H), 3.98 − |
| | | | 3.81 (m, 3H), 3.63 − |
| | | | 3.55 (m, 2H), 3.54 − |
| | | | 3.46 (m, 1H), 3.41 − |
| | | | 3.38 (m, 3H), 2.65 − |
| | | | 2.53 (m, 1H), 2.51 − |
| | | | 2.38 (m, 1H), 2.37 − |
| | | | 2.25 (m, 1H), 2.17 − |
| | | | 2.05 (m, 2H), 2.04 − |
| | | | 1.90 (m, 4H), 1.89 − |
| | | | 1.77 (m, 3H), 1.62 − |
| | | | 1.48 (m, 2H), 1.20 − |
| | | | 1.06 (m, 3H), 0.94 − |
| | | | 0.81 (m, 3H) |
| | 1073 | 838.3 | ¹H NMR (400 MHz, |
| | | | CD₃OD) δ 8.42 − |
| | | | 8.35 (m, 1H), 8.31 − |
| | | | 8.23 (m, 1H), 8.10 |
| | | | (d, J = 13.6 Hz, 2H), |
| | | | 8.03 − 7.94 (m, 1H), |
| | | | 7.71 − 7.61 (m, 1H), |
| | | | 7.37 − 7.29 (m, 2H), |
| | | | 7.25 − 7.12 (m, 3H), |
| | | | 6.18 − 5.99 (m, 1H), |
| | | | 4.79 − 4.71 (m, 1H), |
| | | | 4.68 − 4.61 (m, 1H), |
| | | | 4.61 − 4.55 (m, 1H), |
| | | | 4.47 − 4.31 (m, 2H), |
| | | | 4.26 − 4.19 (m, 3H), |
| | | | 4.18 − 4.03 (m, 3H), |
| | | | 4.01 − 3.95 (m, 1H), |
| | | | 3.94 − 3.87 (m, 1H), |
| | | | 3.84 − 3.77 (m, 1H), |
| | | | 2.37 − 2.26 (m, 1H), |
| | | | 2.18 − 2.06 (m, 2H), |
| | | | 2.04 − 1.88 (m, 5H), |
| | | | 1.87 − 1.77 (m, 2H), |
| | | | 1.62 − 1.46 (m, 2H), |
| | | | 1.26 − 1.18 (m, 3H), |
| | | | 0.92 − 0.81 (m, 3H) |
| | 1074 | 842.2 | ¹H NMR (400 MHz, |
| | | | CD₃OD) δ 8.56 − |
| | | | 8.48 (m, 1H), 8.26 |
| | | | (d, J = 4.0 Hz, 1H), |
| | | | 8.06 − 8.00 (m, 1H), |
| | | | 7.93 − 7.85 (m, 2H), |
| | | | 7.52 − 7.46 (m, 1H), |
| | | | 7.35 − 7.28 (m, 2H), |
| | | | 7.16 (t, J = 9.2 Hz, |
| | | | 3H), 4.86 − 4.80 (m, |
| | | | 1H), 4.74 (d, J = 11.2 |
| | | | Hz, 1H), 4.61 − 4.50 |
| | | | (m, 5H), 4.39 − 4.25 |
| | | | (m, 2H), 4.14 − 4.03 |
| | | | (m, 3H), 3.97 − 3.81 |
| | | | (m, 3H), 3.59 − 3.45 |
| | | | (m, 2H), 2.55 − 2.43 |
| | | | (m, 2H), 2.36 − 2.28 |
| | | | (m, 1H), 2.18 − 2.04 |
| | | | (m, 2H), 2.00 − 1.90 |
| | | | (m, 3H), 1.79 (d, J = |
| | | | 12.0 Hz, 1H), 1.67 − |
| | | | 1.48 (m, 4H), 1.16 − |
| | | | 1.03 (m, 6H), 0.90 − |
| | | | 0.83 (m, 3H) |
| | 1075 | 846.5 | ¹H NMR (400 MHz, |
| | | | CD₃OD) δ 8.56 − |
| | | | 8.47 (m, 1H), 8.26 |
| | | | (d, J = 4.4 Hz, 1H), |
| | | | 8.13 − 8.05 (m, 2H), |
| | | | 7.98 (d, J = 8.0 Hz, |
| | | | 1H), 7.66 (d, J = 8.4 |
| | | | Hz, 1H), 7.38 − 7.29 |
| | | | (m, 2H), 7.23 − 7.13 |
| | | | (m, 3H), 6.19 − 6.02 |
| | | | (m, 1H), 4.95 − 4.88 |
| | | | (m, 1H), 4.74 (d, J = |
| | | | 10.6 Hz, 1H), 4.60 − |
| | | | 4.50 (m, 5H), 4.41 − |
| | | | 4.26 (m, 2H), 4.12 − |
| | | | 4.04 (m, 3H), 4.00 − |
| | | | 3.89 (m, 1H), 3.86 − |
| | | | 3.80 (m, 2H), 2.56 − |
| | | | 2.44 (m, 2H), 2.35 − |
| | | | 2.27 (m, 1H), 2.18 − |
| | | | 2.07 (m, 2H), 2.06 − |
| | | | 1.90 (m, 4H), 1.89 − |
| | | | 1.80 (m, 3H), 1.60 − |
| | | | 1.48 (m, 2H), 1.26 − |
| | | | 1.19 (m, 3H), 0.91 − |
| | | | 0.83 (m, 3H) |
| | 1076 | 846.6 | ¹H NMR (400 MHz, |
| | | | CDCl₃) δ 8.56 − 8.49 |
| | | | (m, 1H), 8.39 − 8.33 |
| | | | (m, 1H), 8.07 − 7.79 |
| | | | (m, 3H), 7.68 − 7.53 |
| | | | (m, 2H), 7.27 − 7.23 |
| | | | (m, 2H), 7.08 − 7.07 |
| | | | (m, 3H), 6.01 − 5.89 |
| | | | (m, 1H), 4.89 − 4.76 |
| | | | (m, 2H), 4.67 − 4.45 |
| | | | (m, 6H), 4.18 − 4.16 |
| | | | (m, 2H), 4.04 − 3.69 |
| | | | (m, 5H), 2.47 − 2.46 |
| | | | (m, 2H), 2.24 − 2.12 |
| | | | (m, 2H), 2.10 − 1.98 |
| | | | (m, 5H), 1.94 − 1.80 |
| | | | (m, 3H), 1.62 − 1.59 |
| | | | (m, 2H), 1.27 − 1.24 |
| | | | (m, 3H), 0.90 − 0.83 |
| | | | (m, 3H) |
| | 1077 | 860.3 | ¹H NMR (400 MHz, |
| | | | CD₃OD) δ 8.49 − |
| | | | 8.57 (m, 1H), 8.27 (s, |
| | | | 1H), 8.08 − 8.16 (m, |
| | | | 2H), 8.00 (d, J = 8.40 |
| | | | Hz, 1H), 7.68 (d, |
| | | | J = 8.00 Hz, 1H), 7.33 − |
| | | | 7.44 (m, 2H), 7.12 − |
| | | | 7.27 (m, 3H), 6.03 − |
| | | | 6.23 (m, 1H), 4.85 (s, |
| | | | 1H), 4.77 (d, J = 11.20 |
| | | | Hz, 1H), 4.50 − 4.66 |
| | | | (m, 5H), 4.27 − 4.44 |
| | | | (m, 2H), 4.05 − 4.16 |
| | | | (m, 3H), 3.82 − 4.02 |
| | | | (m, 3H), 2.44 − 2.58 |
| | | | (m, 2H), 2.34 (m, |
| | | | 1H), 2.06 − 2.22 (m, |
| | | | 2H), 1.89 − 2.03 (m, |
| | | | 3H), 1.82 (d, J = 13.20 |
| | | | Hz, 1H), 1.49 − 1.71 |
| | | | (m, 4H), 1.21 − 1.28 |
| | | | (m, 3H), 1.07 (d, |
| | | | J = 6.40 Hz, 3H), 0.85 − |
| | | | 0.93 (m, 3H) |
| | 1078 | 860.7 | ¹H NMR (400 MHz, |
| | | | CD₃OD) δ 8.47 − |
| | | | 8.58 (m, 1H), 8.26 (s, |
| | | | 1H), 8.08 − 8.15 (m, |
| | | | 2H), 8.01 (d, J-8.40 |
| | | | Hz, 1H), 7.61 − 7.72 |
| | | | (m, 1H), 7.30 − 7.39 |
| | | | (m, 2H), 7.12 − 7.25 |
| | | | (m, 3H), 6.01 − 6.16 |
| | | | (m, 1H), 4.80 − 4.84 |
| | | | (m, 1H), 4.76 (d, |
| | | | J = 11.60 Hz, 1H), |
| | | | 4.50 − 4.63 (m, 5H), |
| | | | 4.26 − 4.42 (m, 2H), |
| | | | 4.04 − 4.13 (m, 3H), |
| | | | 3.95 − 4.01 (m, 2H), |
| | | | 3.77 − 3.85 (m, 1H), |
| | | | 2.45 − 2.54 (m, 2H), |
| | | | 2.27 − 2.38 (m, 1H), |
| | | | 2.04 − 2.19 (m, 2H), |
| | | | 1.89 − 2.01 (m, 3H), |
| | | | 1.80 (d, J = 11.60 Hz, |
| | | | 1H), 1.52 − 1.65 (m, |
| | | | 4H), 1.02 − 1.12 (m, |
| | | | 6H), 0.88 (t, J = 7.20 |
| | | | Hz, 3H) |
| | 1079 | 829.5 | ¹H NMR (400 MHz, |
| | | | CD₃OD) δ 8.40 − |
| | | | 8.30 (m, 1H), 8.17 − |
| | | | 8.06 (m, 1H), 8.03 |
| | | | (d, J = 18.8 Hz, 1H), |
| | | | 7.94 − 7.83 (m, 2H), |
| | | | 7.49 (t, J = 7.2 Hz, |
| | | | 1H), 7.37 − 7.27 (m, |
| | | | 2H), 7.16 (t, J = 9.2 |
| | | | Hz, 3H), 7.13 − 7.03 |
| | | | (m, 1H), 4.99 − 4.94 |
| | | | (m, 1H), 4.71 (t, J = |
| | | | 8.8 Hz, 1H), 4.57 − |
| | | | 4.46 (m, 2H), 4.46 − |
| | | | 4.39 (m, 1H), 4.35 − |
| | | | 4.26 (m, 2H), 4.12 − |
| | | | 4.01 (m, 1H), 3.97 − |
| | | | 3.90 (m, 2H), 3.89 − |
| | | | 3.80 (m, 1H), 3.60 − |
| | | | 3.54 (m, 1H), 3.53 − |
| | | | 3.45 (m, 1H), 3.15 (s, |
| | | | 6H), 2.36 − 2.24 (m, |
| | | | 2H), 2.24 − 2.09 (m, |
| | | | 1H), 2.07 − 2.00 (m, |
| | | | 2H), 1.98 − 1.89 (m, |
| | | | 1H), 1.88 − 1.78 (m, |
| | | | 1H), 1.64 − 1.57 (m, |
| | | | 1H), 1.56 − 1.50 (m, |
| | | | 2H), 1.39 − 1.36 (m, |
| | | | 1H), 1.24 − 1.22 (m, |
| | | | 3H), 1.17 − 1.06 (m, |
| | | | 3H), 0.87 (t, J = 7.2 |
| | | | Hz, 3H) |
| | 1080 | 971.2 | ¹H NMR (400 MHz, |
| | | | CDCl₃) δ 8.11-8.08 |
| | | | (m, 1H), 7.97-7.92 |
| | | | (m, 1H), 7.87-7.85 |
| | | | (m, 1H), 7.70-7.63 |
| | | | (m, 2H), 7.45-7.34 |
| | | | (m, 5H), 4.71-4.59 |
| | | | (m, 2H), 4.28-4.04 |
| | | | (m, 6H), 3.95-3.87 |
| | | | (m, 1H), 3.79-3.70 |
| | | | (m, 1H), 3.44-3.35 |
| | | | (m, 1H), 3.17-3.06 |
| | | | (m, 4H), 2.54-2.43 |
| | | | (m, 1H), 2.32-2.17 |
| | | | (m, 2H), 2.13-1.76 |
| | | | (m, 9H), 1.27-1.17 |
| | | | (m, 18H), 1.10-1.02 |
| | | | (m, 1H), 0.80-0.66 |
| | | | (m, 1H) |
| | 1081 | 971.3 | ¹H NMR (400 MHz, |
| | | | CDCl₃) δ 8.12-8.07 |
| | | | (m, 1H), 7.97-7.91 |
| | | | (m, 1H), 7.88-7.85 |
| | | | (m, 1H), 7.69-7.62 |
| | | | (m, 2H), 7.44-7.33 |
| | | | (m, 5H), 4.75-4.45 |
| | | | (m, 3H), 4.27-4.11 |
| | | | (m, 4H), 3.96-3.78 |
| | | | (m, 2H), 3.67 (d, J = |
| | | | 9.5 Hz, 1H), 3.56- |
| | | | 3.48 (m, 1H), 3.16- |
| | | | 3.06 (m, 4H), 2.48- |
| | | | 2.33 (m, 1H), 2.28- |
| | | | 2.16 (m, 2H), 2.09- |
| | | | 1.87 (m, 7H), 1.84- |
| | | | 1.72 (m, 2H), 1.24- |
| | | | 1.19 (m, 18H), 1.18- |
| | | | 1.10 (m, 1H), 0.83- |
| | | | 0.67 (m, 1H) |
| | 1082 | 960.3 | ¹H NMR (400 MHz, |
| | | | CDCl₃) δ 8.08-7.96 |
| | | | (m, 1H), 7.93-7.80 |
| | | | (m, 2H), 7.64-7.56 |
| | | | (m, 1H), 7.27-7.17 |
| | | | (m, 5H), 5.33-5.02 |
| | | | (m, 1H), 4.73-4.51 |
| | | | (m, 1H), 4.38-4.08 |
| | | | (m, 5H), 4.05-3.91 |
| | | | (m, 1H), 3.72-3.48 |
| | | | (m, 2H), 3.27-3.05 |
| | | | (m, 4H), 2.47-2.41 |
| | | | (m, 4H), 2.23-1.62 |
| | | | (m, 18H), 0.95 (d, J = |
| | | | 6.4 Hz, 12H), 0.62- |
| | | | 0.49 (m, 2H) |
| | 1083 | 980 | ¹H NMR (400 MHz, |
| | | | DMSO-d₆) δ 11.61 |
| | | | (s, 1H), 8.97-8.85 |
| | | | (m, 1H), 8.39-8.29 |
| | | | (m, 1H), 8.25-8.19 |
| | | | (m, 1H), 8.16 (s, 1H), |
| | | | 7.59 (d, J = 8.4 Hz, |
| | | | 1H), 7.43-7.34 (m, |
| | | | 1H), 6.43-6.33 (m, |
| | | | 1H), 6.30-6.19 (m, |
| | | | 1H), 4.96 (s, 1H), |
| | | | 4.62-4.49 (m, 1H), |
| | | | 4.37-4.10 (m, 6H), |
| | | | 3.60-3.53 (m, 5H), |
| | | | 3.20 (s, 6H), 3.15 (t, |
| | | | J = 6.3 Hz, 4H), 2.82 |
| | | | (t, J = 5.9 Hz, 4H), |
| | | | 2.28-1.51 (m, 12H) |
| | 1084 | 871 | ¹H NMR (400 MHz, |
| | | | CDCl₃) δ 8.92 (s, |
| | | | 1H), 8.80-8.64 (m, |
| | | | 1H), 8.42-8.00 (m, |
| | | | 1H), 7.88-7.72 (m, |
| | | | 4H), 7.66-7.59 (m, |
| | | | 1H), 7.40-7.34 (m, |
| | | | 1H), 4.81-4.45 (m, |
| | | | 4H), 4.27-4.13 (m, |
| | | | 1H), 4.10-3.82 (m, |
| | | | 11H), 3.31 (d, J = |
| | | | 21.6 Hz, 2H), 3.17- |
| | | | 2.99 (m, 4H), 2.33- |
| | | | 1.83 (m, 12H), 0.94 |
| | | | (d, J = 6.6 Hz, 12H) |
| | 1085 | 990.4 | ¹H NMR (400 MHz, |
| | | | CDCl₃) δ 8.08 (s, |
| | | | 1H), 8.04 (s, 1H), |
| | | | 7.95 − 7.81 (m, 2H), |
| | | | 7.66 − 7.58 (m, 1H), |
| | | | 7.45 − 7.39 (m, 1H), |
| | | | 6.65 − 6.53 (m, 1H), |
| | | | 6.33 − 6.22 (m, 1H), |
| | | | 5.75 − 5.60 (m, 2H), |
| | | | 5.17 − 5.08 (m, 1H), |
| | | | 4.90 − 4.80 (m, 1H), |
| | | | 4.62 − 4.46 (m, 2H), |
| | | | 4.39 − 4.18 (m, 3H), |
| | | | 4.02 − 3.87 (m, 3H), |
| | | | 3.84 − 3.77 (m, 1H), |
| | | | 3.57 − 3.47 (m, 3H), |
| | | | 3.39 − 3.24 (m, 1H), |
| | | | 3.21 − 3.04 (m, 3H), |
| | | | 2.41 (d, J = 7.1 Hz, |
| | | | 2H), 2.28 − 2.03 (m, |
| | | | 7H), 2.00 − 1.82 (m, |
| | | | 6H), 1.79 − 1.70 (m, |
| | | | 3H), 0.96 − 0.91 (m, |
| | | | 6H) |
| | 1086 | 934.9 | ¹H NMR (400 MHz, |
| | | | DMSO-d₆) δ 8.74 − |
| | | | 8.62 (m, 2H), 8.31 |
| | | | 8.26 (m, 1H), 8.22 − |
| | | | 8.08 (m, 4H), 7.79 |
| | | | 7.68 (m, 2H), 5.15 − |
| | | | 4.91 (m, 2H), 4.67 |
| | | | 4.43 (m, 2H), 4.43 |
| | | | 4.28 (m, 2H), 4.25 − |
| | | | 4.10 (m, 5H), 4.08 − |
| | | | 3.88 (m, 2H), 3.85 |
| | | | 3.76 (m, 2H), 3.61 |
| | | | 3.41 (m, 3H), 3.20 − |
| | | | 3.11 (m, 4H), 2.47 − |
| | | | 2.39 (m, 4H), 2.25 − |
| | | | 1.54 (m, 14H), 0.90 − |
| | | | 0.85 (m, 12H) |
| | 1087 | 914.0 | ¹H NMR (400 MHz, |
| | | | CDCl₃) δ 8.61 − 8.39 |
| | | | (m, 1H), 8.33 − 8.18 |
| | | | (m, 1H), 8.09 − 7.68 |
| | | | (m, 6H), 7.48 − 7.37 |
| | | | (m, 1H), 6.69 − 6.54 |
| | | | (m, 1H), 6.31 − 6.21 |
| | | | (m, 1H), 5.78 − 5.64 |
| | | | (m, 4H), 5.23 − 5.10 |
| | | | (m, 1H), 4.95 − 4.88 |
| | | | (m, 2H), 4.60 − 4.47 |
| | | | (m, 2H), 4.41 − 4.30 |
| | | | (m, 1H), 3.97 − 3.80 |
| | | | (m, 2H), 3.67 − 3.46 |
| | | | (m, 2H), 3.36 − 3.18 |
| | | | (m, 1H), 2.25 − 2.13 |
| | | | (m, 4H), 2.07 − 1.90 |
| | | | (m, 4H), 1.82 − 1.63 |
| | | | (m, 4H), 1.29 (d, J = |
| | | | 6.2 Hz, 12H) |
| | 1088 | 914.1 | ¹H NMR (400 MHz, |
| | | | CDCl₃) δ 8.51 − 8.42 |
| | | | (m, 1H), 8.30 − 8.20 |
| | | | (m, 1H), 8.08 − 7.86 |
| | | | (m, 4H), 7.77 − 7.68 |
| | | | (m, 1H), 7.47 − 7.30 |
| | | | (m, 1H), 6.77 − 6.51 |
| | | | (m, 1H), 6.31 − 6.19 |
| | | | (m, 1H), 5.79 − 5.63 |
| | | | (m, 4H), 5.24 − 5.10 |
| | | | (m, 1H), 4.96 − 4.84 |
| | | | (m, 2H), 4.60 − 4.49 |
| | | | (m, 1H), 4.41 − 4.15 |
| | | | (m, 3H), 4.11 − 3.92 |
| | | | (m, 1H), 3.78 − 3.57 |
| | | | (m, 2H), 3.19 − 3.08 |
| | | | (m, 1H), 2.32 − 1.60 |
| | | | (m, 14H), 1.32 − 1.26 |
| | | | (m, 12H) |
| | 1089 | 889.2 | ¹H NMR (400 MHz, |
| | | | CDCl₃) δ 8.62 (s, |
| | | | 1H), 8.02 − 7.87 (m, |
| | | | 3H), 7.86 − 7.79 (m, |
| | | | 1H), 7.76 − 7.66 (m, |
| | | | 1H), 7.54 (d, J = 8.3 |
| | | | Hz, 1H), 7.51 − 7.33 |
| | | | (m, 1H), 5.84 (dd, J = |
| | | | 44.4, 7.4 Hz, 1H), |
| | | | 4.75 − 4.37 (m, 4H), |
| | | | 4.25 − 3.84 (m, 8H), |
| | | | 3.16 − 3.03 (m, 4H), |
| | | | 2.47 − 2.37 (m, 4H), |
| | | | 2.34 − 1.72 (m, 13H), |
| | | | 0.94 (d, J = 6.6 Hz, |
| | | | 12H) |
| | 1090 | 958.0 | ¹H NMR (400 MHz, |
| | | | CDCl₃) δ 8.01 − 7.82 |
| | | | (m, 3H), 7.56 − 7.36 |
| | | | (m, 2H), 6.65 − 6.55 |
| | | | (m, 1H), 6.31 − 6.23 |
| | | | (m, 1H), 5.92 − 5.75 |
| | | | (m, 1H), 5.16 − 5.07 |
| | | | (m, 1H), 4.62 − 4.47 |
| | | | (m, 2H), 4.41 − 4.27 |
| | | | (m, 1H), 4.18 − 3.64 |
| | | | (m, 7H), 3.57 − 3.31 |
| | | | (m, 2H), 3.16 − 3.04 |
| | | | (m, 4H), 2.47 − 2.38 |
| | | | (m, 4H), 2.22 − 1.63 |
| | | | (m, 14H), 0.99 − 0.88 |
| | | | (m, 12H) |
| | 1091 | 924.2 | ¹H NMR (400 MHz, |
| | | | DMSO-d₆) δ 11.62 |
| | | | (s, 1H), 8.99 − 8.88 |
| | | | (m, 1H), 8.39 − 8.30 |
| | | | (m, 1H), 8.26 − 8.17 |
| | | | (m, 2H), 7.66 (d, J = |
| | | | 8.5 Hz, 1H), 7.46 − |
| | | | 7.34 (m, 3H), 7.26 (t, |
| | | | J = 7.4 Hz, 1H), 7.16 |
| | | | (d, J = 8.0 Hz, 2H), |
| | | | 6.42 − 6.33 (m, 1H), |
| | | | 6.31 − 6.16 (m, 1H), |
| | | | 5.00 − 4.86 (m, 2H), |
| | | | 4.61 − 4.50 (m, 1H), |
| | | | 4.35 − 4.07 (m, 4H), |
| | | | 4.02 − 3.41 (m, 5H), |
| | | | 3.26 − 3.15 (m, 1H), |
| | | | 3.10 (t, J = 6.3 Hz, |
| | | | 2H), 2.33 − 2.19 (m, |
| | | | 1H), 2.09 − 1.77 (m, |
| | | | 8H), 1.68 − 1.45 (m, |
| | | | 3H), 1.07 (s, 6H) |
| | 1092 | 971.4 | ¹H NMR (400 MHz, |
| | | | CDCl₃) δ 8.55 − 8.39 |
| | | | (m, 1H), 8.19 (d, J = |
| | | | 10.2 Hz, 1H), 8.06 − |
| | | | 7.84 (m, 3H), 7.78 − |
| | | | 7.67 (m, 1H), 7.64 |
| | | | 7.54 (m, 1H), 7.41 − |
| | | | 7.30 (m, 1H), 6.75 − |
| | | | 6.46 (m, 1H), 6.46 |
| | | | 6.19 (m, 1H), 5.28 − |
| | | | 5.09 (m, 1H), 4.58 − |
| | | | 4.32 (m, 2H), 4.30 − |
| | | | 4.13 (m, 4H), 4.13 − |
| | | | 3.86 (m, 1H), 3.67 − |
| | | | 3.35 (m, 2H), 3.21 − |
| | | | 3.08 (m, 4H), 2.47 − |
| | | | 2.36 (m, 4H), 2.27 − |
| | | | 1.88 (m, 13H), 1.88 − |
| | | | 1.76 (m, 2H), 1.72 − |
| | | | 1.58 (m, 2H), 1.36 − |
| | | | 1.26 (m, 1H), 1.02 − |
| | | | 0.83 (m, 12H), 0.62 − |
| | | | 0.35 (m, 2H) |
| | 1093 | 962.2 | ¹H NMR (400 MHz, |
| | | | CDCl₃) δ 11.62 (s, |
| | | | 1H), 9.00 − 8.86 (m, |
| | | | 1H), 8.38 − 8.30 (m, |
| | | | 1H), 8.25 − 8.18 (m, |
| | | | 1H), 8.14 (s, 1H), |
| | | | 7.57 (d, J = 8.6 Hz, − |
| | | | 106.65 (d, J = 121.2 |
| | | | Hz).), 7.39 (d, J = 6.6 |
| | | | Hz, 1H), 6.39 − 6.34 |
| | | | (m, 1H), 6.28 − 6.21 |
| | | | (m, 1H), 5.70 (d, J = |
| | | | 13.1 Hz, 2H), 5.43 − |
| | | | 5.33 (m, 1H), 5.00 − |
| | | | 4.89 (m, 1H), 4.80 (t, |
| | | | J = 7.0 Hz, 2H), 4.51 |
| | | | (dd, J = 39.1, 7.5 Hz, |
| | | | 3H), 4.33 − 4.15 (m, |
| | | | 4H), 3.95 − 3.44 (m, |
| | | | 5H), 3.13 (t, J = 6.2 |
| | | | Hz, 2H), 2.43 (d, J = |
| | | | 7.1 Hz, 2H), 2.32 − |
| | | | 2.18 (m, 1H), 2.05 − |
| | | | 1.95 (m, 2H), 1.92 − |
| | | | 1.67 (m, 7H), 1.66 − |
| | | | 1.46 (m, 3H), 0.89 − |
| | | | 0.83 (m, 6H) |
| | 1094 | 975.9 | ¹H NMR (400 MHz, |
| | | | CDCl₃) δ 8.17 − 7.79 |
| | | | (m, 2H), 7.66 − 7.56 |
| | | | (m, 1H), 7.50 − 7.36 |
| | | | (m, 1H), 6.70 − 6.51 |
| | | | (m, 1H), 6.41 − 6.24 |
| | | | (m, 1H), 5.76 − 5.57 |
| | | | (m, 1H), 5.31 − 5.20 |
| | | | (m, 1H), 5.17 − 5.02 |
| | | | (m, 1H), 4.57 − 4.43 |
| | | | (m, 1H), 4.39 − 4.18 |
| | | | (m, 3H), 4.13 − 3.82 |
| | | | (m, 6H), 3.80 − 3.30 |
| | | | (m, 3H), 3.25 − 3.07 |
| | | | (m, 3H), 2.42 (d, J = |
| | | | 7.1 Hz, 2H), 2.27 |
| | | | 2.06 (m, 9H), 2.01 − |
| | | | 1.90 (m, 2H), 1.83 − |
| | | | 1.75 (m, 1H), 1.70 − |
| | | | 1.60 (m, 2H), 1.46 − |
| | | | 1.37 (m, 1H), 0.98 − |
| | | | 0.90 (m, 6H) (TFA |
| | | | salt) |
| | 1095 | 894.2 | ¹H NMR (400 MHz, |
| | | | CDCl₃) δ 8.94 − 8.78 |
| | | | (m, 3H), 8.33 (d, J = |
| | | | 16.2 Hz, 1H), 8.26 − |
| | | | 8.19 (m, 1H), 8.14 |
| | | | (d, J = 10.9 Hz, 1H), |
| | | | 7.64 − 7.55 (m, 1H), |
| | | | 7.47 − 7.37 (m, 1H), |
| | | | 5.70 (d, J = 13.1 Hz, |
| | | | 2H), 5.43 − 5.35 (m, |
| | | | 1H), 4.84 − 4.40 (m, |
| | | | 8H), 4.31 − 3.94 (m, |
| | | | 6H), 3.13 (t, J = 6.0 |
| | | | Hz, 2H), 2.46 − 2.40 |
| | | | (m, 2H), 2.26 − 2.14 |
| | | | (m, 1H), 2.07 − 1.92 |
| | | | (m, 3H), 1.89 − 1.63 |
| | | | (m, 7H), 0.90 − 0.81 |
| | | | (m, 6H) |
| | 1096 | 861.1 | ¹H NMR (400 MHz, |
| | | | CDCl₃) δ 8.64 − 8.53 |
| | | | (m, 2H), 8.00 − 7.94 |
| | | | (m, 1H), 7.90 (d, J = |
| | | | 9.0 Hz, 1H), 7.84 (d, |
| | | | J = 13.0 Hz, 1H), |
| | | | 7.77 − 7.70 (m, 1H), |
| | | | 7.70 − 7.62 (m, 1H), |
| | | | 7.53 (d, J = 8.7 Hz, |
| | | | 1H), 7.45 − 7.30 (m, |
| | | | 1H), 5.96 − 5.76 (m, |
| | | | 1H), 5.69 − 5.58 (m, |
| | | | 2H), 5.04 − 4.90 (m, |
| | | | 1H), 4.71 − 4.37 (m, |
| | | | 4H), 4.23 − 4.06 (m, |
| | | | 3H), 3.99 − 3.87 (m, |
| | | | 2H), 3.09 (q, J = 6.6 |
| | | | Hz, 2H), 2.43 − 2.36 |
| | | | (m, 2H), 2.31 − 2.17 |
| | | | (m, 2H), 2.09 − 2.02 |
| | | | (m, 4H), 1.97 − 1.90 |
| | | | (m, 2H), 1.86 − 1.68 |
| | | | (m, 4H), 1.32 (d, J = |
| | | | 6.2 Hz, 6H), 0.97 − |
| | | | 0.89 (m, 6H) |
| | 1097 | 976.0 | ¹H NMR (400 MHz, |
| | | | CDCl₃) δ 8.11 − 7.86 |
| | | | (m, 3H), 7.84 − 7.30 |
| | | | (m, 3H), 6.61 − 6.47 |
| | | | (m, 1H), 6.24 − 6.16 |
| | | | (m, 1H), 5.75 − 5.59 |
| | | | (m, 2H), 5.30 − 5.06 |
| | | | (m, 2H), 4.63 − 4.47 |
| | | | (m, 2H), 4.38 − 4.16 |
| | | | (m, 3H), 4.13 − 3.57 |
| | | | (m, 7H), 3.54 − 3.04 |
| | | | (m, 4H), 2.45 − 2.38 |
| | | | (m, 2H), 2.26 − 2.09 |
| | | | (m, 7H), 1.95 − 1.83 |
| | | | (m, 6H), 1.71 − 1.60 |
| | | | (m, 2H), δ 1.01 − |
| | | | 0.86 (m, 6H) |
| | 1098 | 908.4 | ¹H NMR (400 MHz, |
| | | | CDCl₃) δ 8.76 (s, |
| | | | 2H), 8.12 − 8.02 (m, |
| | | | 1H), 7.98 − 7.80 (m, |
| | | | 2H), 7.79 − 7.59 (m, |
| | | | 2H), 7.26 − 7.18 (m, |
| | | | 1H), 5.75 − 5.60 (m, |
| | | | 2H), 5.32 − 5.20 (m, |
| | | | 1H), 4.96 − 4.82 (m, |
| | | | 1H), 4.74 − 4.12 (m, |
| | | | 8H), 4.00 − 3.79 (m, |
| | | | 5H), 3.18 − 3.03 (m, |
| | | | 2H), 2.42 (d, J = 7.1 |
| | | | Hz, 2H), 2.29 − 2.03 |
| | | | (m, 8H), 1.98 − 1.72 |
| | | | (m, 5H), 0.94 (d, J = |
| | | | 6.6 Hz, 6H) |
| | 1099 | 908.0 | ¹H NMR (400 MHz, |
| | | | CDCl₃) δ 8.88 − 8.68 |
| | | | (m, 2H), 8.12 − 8.03 |
| | | | (m, 1H), 7.99 − − 7.83 |
| | | | (m, 2H), 7.82 − 7.73 |
| | | | (m, 1H), 7.68 − − 7.58 |
| | | | (m, 1H), 7.30 − 7.27 |
| | | | (m, 1H), 5.76 − 5.58 |
| | | | (m, 2H), 5.31 − 5.21 |
| | | | (m, 1H), 4.96 − 4.83 |
| | | | (m, 1H), 4.72 − 4.14 |
| | | | (m, 8H), 4.00 − 3.83 |
| | | | (m, 5H), 3.18 − 3.05 |
| | | | (m, 2H), 2.46 − 2.37 |
| | | | (m, 2H), 2.30 − 1.99 |
| | | | (m, 9H), 1.98 − 1.69 |
| | | | (m, 4H), 1.01 − 0.86 |
| | | | (m, 6H) (TFA salt) |
| | 1100 | 942.3 | ¹H NMR (400 MHz, |
| | | | CDCl₃) δ 8.63 − 8.39 |
| | | | (m, 1H), 8.35 − 8.16 |
| | | | (m, 1H), 8.09 − 7.63 |
| | | | (m, 5H), 7.51 − 7.37 |
| | | | (m, 1H), 6.75 − 6.61 |
| | | | (m, 1H), 6.38 − 6.28 |
| | | | (m, 1H), 5.75 − 5.56 |
| | | | (m, 2H), 5.21 − 5.08 |
| | | | (m, 1H), 4.96 − 4.86 |
| | | | (m, 1H), 4.65 − 4.42 |
| | | | (m, 2H), 4.38 − 4.29 |
| | | | (m, 1H), 4.29 − 4.09 |
| | | | (m, 2H), 3.98 − 3.43 |
| | | | (m, 4H), 3.36 − 3.05 |
| | | | (m, 3H), 2.42 − 2.36 |
| | | | (m, 2H), 2.26 − 1.61 |
| | | | (m, 13H), 1.30 (d, J = |
| | | | 6.2 Hz, 6H), 0.92 (d, |
| | | | J = 6.6 Hz, 6H) |
| | 1101 | 954.4 | ¹H NMR (400 MHz, |
| | | | CDCl₃) δ 8.37 − 8.31 |
| | | | (m, 1H), 8.09 (s, 1H), |
| | | | 7.91 − 7.79 (m, 3H), |
| | | | 7.65 (d, J = 8.7 Hz, |
| | | | 1H), 7.54 − 7.46 (m, |
| | | | 1H), 7.24 − 7.12 (m, |
| | | | 5H), 5.16 − 5.05 (m, |
| | | | 1H), 4.54 − 4.17 (m, |
| | | | 3H), 4.18 − 4.05 (m, |
| | | | 4H), 3.72 − 3.55 (m, |
| | | | 1H), 3.54 − 3.40 (m, |
| | | | 1H), 3.13 − 2.96 (m, |
| | | | 4H), 2.37 − 2.28 (m, |
| | | | 5H), 2.18 − 1.48 (m, |
| | | | 17H), 0.84 (d, J = 6.7 |
| | | | Hz, 12H), 0.53 − 0.37 |
| | | | (m, 2H) |
| | 1102 | 971.2 | ¹H NMR (400 MHz, |
| | | | CDCl₃) δ 8.84 − 8.52 |
| | | | (m, 2H), 8.19 − 7.85 |
| | | | (m, 4H), 7.83 − 7.61 |
| | | | (m, 2H), 7.57 − 7.35 |
| | | | (m, 1H), 5.10 − 4.51 |
| | | | (m, 4H), 4.49 − 3.86 |
| | | | (m, 12H), 3.80 − 3.70 |
| | | | (m, 4H), 3.65 − 3.55 |
| | | | (m, 4H), 3.39 (s, 6H), |
| | | | 3.25 − 3.08 (m, 4H), |
| | | | 2.36 − 1.67 (m, 10H). |
| | 1103 | 898.4 | ¹H NMR (400 MHz, |
| | | | DMSO-d₆) δ 10.24 |
| | | | (s, 1H), 8.37 (d, J = |
| | | | 7.2 Hz, 1H), 7.79 |
| | | | 7.67 (m, 4H), 7.67 − |
| | | | 7.55 (m, 6H), 7.51 − |
| | | | 7.38 (m, 3H), 7.37 − |
| | | | 7.27 (m, 1H), 6.91 |
| | | | (d, J = 15.8 Hz, 1H), |
| | | | 5.01 − 4.85 (m, 1H), |
| | | | 4.48 (t, J = 8.5 Hz, |
| | | | 1H), 4.38 − 4.25 (m, |
| | | | 1H), 4.24 − 4.05 (m, |
| | | | 4H), 3.21 − 3.04 (m, |
| | | | 4H), 2.57 (t, J = 7.3 |
| | | | Hz, 4H), 2.30 − 2.19 |
| | | | (m, 1H), 2.17 − 2.04 |
| | | | (m, 1H), 2.04 − 1.72 |
| | | | (m, 6H), 1.71 − 1.50 |
| | | | (m, 8H), 0.88 (t, J = |
| | | | 7.4 Hz, 6H) |
| | 1104 | 866.4 | ¹H NMR (400 MHz, |
| | | | DMSO-d₆) δ 10.28 − |
| | | | 10.14 (m, 1H), 8.42 − |
| | | | 8.25 (m, 1H), 7.75 − |
| | | | 7.67 (m, 4H), 7.67 − |
| | | | 7.60 (m, 4H), 7.59 − |
| | | | 7.52 (m, 2H), 7.49 − |
| | | | 7.38 (m, 3H), 7.37 − |
| | | | 7.27 (m, 1H), 6.97 − |
| | | | 6.76 (m, 1H), 5.81 − |
| | | | 5.58 (m, 4H), 4.98 |
| | | | 4.85 (m, 1H), 4.54 − |
| | | | 4.39 (m, 1H), 4.37 − |
| | | | 4.23 (m, 1H), 2.30 |
| | | | 2.18 (m, 1H), 2.17 − |
| | | | 2.04 (m, 1H), 2.03 |
| | | | 1.83 (m, 4H), 1.83 − |
| | | | 1.71 (m, 2H), 1.71 − |
| | | | 1.57 (m, 3H), 1.57 − |
| | | | 1.46 (m, 1H), 1.15 |
| | | | (d, J = 6.5 Hz, 18H). |
| | 1105 | 922.2 | ¹H NMR (400 MHz, |
| | | | CDCl₃) δ 8.81 − 8.72 |
| | | | (m, 2H), 8.11 − 8.04 |
| | | | (m, 1H), 7.97 − 7.89 |
| | | | (m, 1H), 7.88 − 7.81 |
| | | | (m, 1H), 7.80 − 7.73 |
| | | | (m, 1H), 7.66 − 7.60 |
| | | | (m, 1H), 7.26 − 7.22 |
| | | | (m, 1H), 5.75 − 5.62 |
| | | | (m, 2H), 4.95 − 4.79 |
| | | | (m, 2H), 4.69 − 4.55 |
| | | | (m, 3H), 4.29 − 4.18 |
| | | | (m, 3H), 3.98 − 3.88 |
| | | | (m, 3H), 3.60 − 3.48 |
| | | | (m, 2H), 3.17 − 3.06 |
| | | | (m, 2H), 2.42 (d, J = |
| | | | 7.1 Hz, 2H), 2.32 |
| | | | 2.08 (m, 4H), 2.07 − |
| | | | 1.91 (m, 8H), 1.82 − |
| | | | 1.69 (m, 4H), 1.56 − |
| | | | 1.40 (m, 1H), 0.97 − |
| | | | 0.92 (m, 6H) |
| | 1106 | 911.2 | ¹H NMR (400 MHz, |
| | | | CD₃OD) δ 8.65 − |
| | | | 8.36 (m, 2H), 8.25 − |
| | | | 7.92 (m, 4H), 7.67 |
| | | | (d, J = 8.6 Hz, 1H), |
| | | | 7.54 − 7.38 (m, 1H), |
| | | | 5.01 − 4.40 (m, 5H), |
| | | | 4.36 − 4.18 (m, 4H), |
| | | | 4.15 − 3.96 (m, 3H), |
| | | | 3.72 − 3.57 (m, 4H), |
| | | | 3.32 − 3.29 (m, 6H), |
| | | | 3.25 − 3.12 (m, 4H), |
| | | | 2.87 − 2.77 (m, 4H), |
| | | | 2.40 − 2.26 (m, 1H), |
| | | | 2.22 − 1.78 (m, 9H). |

### General Scheme 3: Acids Table

The following compounds in Table 111 were prepared according to the representative procedure described above for the synthesis of (Difluoro(2-(((3S,6S,9aS)-3-(methyl(phenyl)carbamoyl)-5-oxooctahydro-1H-pyrrolo[1,2-a]azepin-6-yl)carbamoyl)benzo[b]thiophen-5-yl)methyl)phosphonic acid (111) utilizing the appropriate starting materials and modifications.

TABLE 111

| Com- | | | LCMS | |
|---|---|---|---|---|
| pound | Name | Structure | [M + H]⁺ | NMR |
| 1107 | ((2- (((3S,6S,10aS)- 3-((1-(1H- pyrazolo[3,4- b]pyridin-3- yl)ethyl)car- bamoyl)-5- oxodecahydro pyrrolo[1,2- a]azocin-6- yl)carbamoyl) | 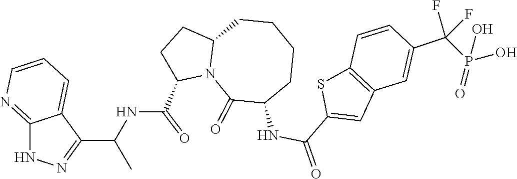 [SMILES: CC(NC(=O)[C@@H]1CC[C@@H]2CCCC[C@H](NC(=O)c3cc4cc(C(F)(F)P(=O)(O)O)ccc4s3)C(=O)N21)c1n[nH]c2ncccc12] | 661.3 | ¹H NMR (400 MHz, CD₃OD) δ 8.55-8.27 (m, 2H), 8.21-8.06 (m, 2H), 7.97- 7.88 (m, 1H), 7.78-7.68 (m, 1H), 7.23-7.12 (m, 1H), 5.55- 5.47 (m, 1H), 5.04 (d, J = 1.6 Hz, |
| | benzo[b]thio- | | | 1H), 4.52- |
| | phen-5- | | | 4.36 (m, 2H), |
| | yl)difluoro- | | | 2.26-2.12 (m, |
| | methyl) | | | 2H), 2.08-1.87 |
| | phosphonic | | | (m, 6H), 1.86- |
| | acid | | | 1.73 (m, 2H), |
| | | | | 1.73-1.57 |
| | | | | (m, 5H) |
| 1108 | ((2- (((3S,6S,10aS)- 3-(((1H- pyrazolo[3,4- b]pyridin-5- yl)methyl)car- bamoyl)-5- oxodecahydro pyrrolo[1,2- a]azocin-6- | 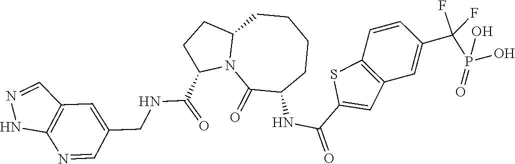 [SMILES: O=C(N[C@H]1CCCC[C@H]2CC[C@@H](C(=O)NCc3cnc4[nH]ncc4c3)N2C1=O)c1cc2cc(C(F)(F)P(=O)(O)O)ccc2s1] | 647.2 | ¹H NMR (400 MHz, CD₃OD) δ 8.53-8.56 (m, 1H), 8.24 (d, J = 2.00 Hz, 1H), 8.17 (s, 1H), 8.13 (s, 1H), 8.10 (s, 1H), 7.93-7.98 (m, 1H), 7.69-7.74 |
| | yl)carbamoyl) | | | (m, 1H), 5.06 |
| | benzo[b]thio- | | | (m, 1H), 4.60- |
| | phen-5- | | | 4.69 (m, 1H), |
| | yl)difluoro- | | | 4.46 (m, 3H), |
| | methyl) | | | 2.18-2.28 (m, |
| | phosphonic | | | 2H), 1.98 (s, |
| | acid | | | 4H) 1.75-1.89 |
| | | | | (m, 2H) 1.66 (s, |
| | | | | 2H), 1.30 (s, |
| | | | | 2H) |
| 1109 | (difluoro(2- (((3S,6S,10aS)- 3-(((rel- trans)-4- fluorotetra- hydrofuran-3- yl)carbamoyl)- 5- oxodecahydro pyrrolo[1,2- a]azocin-6- | 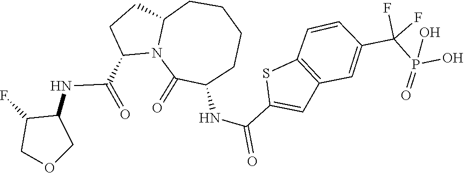 | 604.0 | ¹H NMR (400 MHz, CD₃OD) δ 8.18 (s, 1H), 8.13 (m, 1H), 7.93 (m, 1H), 7.74 (d, J = 8.8 Hz, 1H), 5.17- 4.95 (m, 2H), 4.45-4.31 (m, 3H), 4.12-4.05 (m, 1H), 4.04- |
| | yl)carbamoyl) | | | 3.86 (m, 2H), |
| | benzo[b]thio- | | | 3.77-3.69 (m, |
| | phen-5- | | | 1H), 2.27-2.16 |
| | yl)methyl) | | | (m, 2H), 2.08- |
| | phosphonic | | | 1.92 (m, 6H), |
| | acid | | | 1.87-1.77 (m, |
| | | | | 2H), 1.73-1.60 |
| | | | | (m, 2H) |
| 1110 | ((2- (((3S,6S,10aS)- 3-(3-(4- cyano-2- methylphenyl) azetidine-1- carbonyl)-5- oxodecahydro pyrrolo[1,2- a]azocin-6- yl)carbamoyl) benzo[b]thiop hen-5- yl)difluoro- | 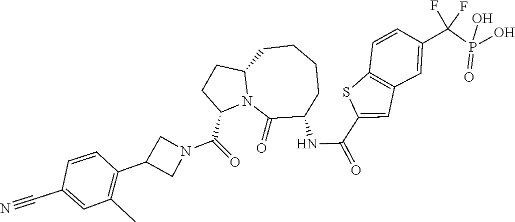 [SMILES: Cc1cc(C#N)ccc1C1CN(C(=O)[C@@H]2CC[C@@H]3CCCC[C@H](NC(=O)c4cc5cc(C(F)(F)P(=O)(O)O)ccc5s4)C(=O)N32)C1] | 671.1 | ¹H NMR (400 MHz, CD₃OD) δ 8.23-8.05 (m, 2H), 7.94-7.88 (m, 1H), 7.73 (t, J = 7.2 Hz, 1H), 7.66-7.55 (m, 2H), 7.55-7.46 (m, 1H), 5.06- 4.90 (m, 2H), 4.69-4.64 (m, 1H), 4.53-4.47 (m, 1H), 4.46- 4.39 (m, 1H), |
| | methyl) | | | 4.39-4.26 (m, |
| | phosphonic | | | 1H), 4.26-4.17 |
| | acid | | | (m, 1H), 4.12- |
| | | | | 4.00 (m, 1H), |
| | | | | 2.33-2.18 (m, |
| | | | | 5H), 2.09-1.91 |
| | | | | (m, 6H), 1.89- |
| | | | | 1.75 (m, 2H), |
| | | | | 1.74-1.57 (m, |
| | | | | 2H) |
| 1111 | (difluoro(2- (((3S,6S,10aS)- 3-(((rel- trans)-4- fluorotetra- hydrofuran-3- yl)carbamoyl)- 5- oxodecahydro pyrrolo[1,2- a]azocin-6- | 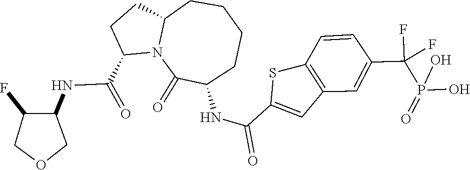 [SMILES: O=C(N[C@H]1CCCC[C@H]2CC[C@@H](C(=O)N[C@H]3COC[C@H]3F)N2C1=O)c1cc2cc(C(F)(F)P(=O)(O)O)ccc2s1] | 604.0 | ¹H NMR (400 MHz, CD₃OD) δ 8.19 (s, 1H), 8.12 (s, 1H), 7.92 (d, J = 8.8 Hz, 1H), 7.76 (d, J = 8.4 Hz, 1H), 5.11-5.01 (m, 2H), 4.60- 4.36 (m, 3H), 4.15-4.03 (m, |
| | yl)carbamoyl) | | | 2H), 4.03-3.95 |
| | benzo[b]thio- | | | (m, 1H), 3.70- |
| | phen-5- | | | 3.52 (m, 1H), |
| | yl)methyl) | | | 2.31-2.17 (m, |
| | phosphonic | | | 2H), 2.11-1.91 |
| | acid | | | (m, 6H), 1.87- |
| | | | | 1.77 (m, 2H), |
| | | | | 1.74-1.58 (m, |
| | | | | 2H) |
| 1112 | ((2- (((3S,6S,10aS)- 3-(((3H- imidazo[4,5- b]pyridin-6- yl)methyl)car- bamoyl)-5- oxodecahydro pyrrolo[1,2- a]azocin-6- yl)carbamoyl) | 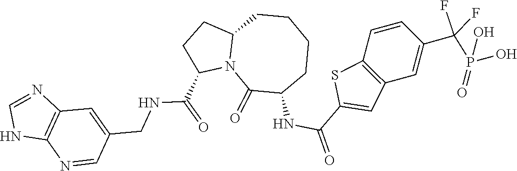 [SMILES: O=C(N[C@H]1CCCC[C@H]2CC[C@@H](C(=O)NCc3cnc4[nH]cnc4c3)N2C1=O)c1cc2cc(C(F)(F)P(=O)(O)O)ccc2s1] | 647.2 | ¹H NMR (400 MHz, CD₃OD) δ 8.48-8.38 (m, 2H), 8.18-8.09 (m, 2H), 8.04 (s, 1H), 7.89 (d, J = 8.4 Hz, 1H), 7.72 (d, J = 8.8 Hz, 1H), 5.01 (d, J = 10.4 Hz, 1H), 4.67 (d, J = |
| | benzo[b]thio- | | | 15.2 Hz, 1H), |
| | phen-5- | | | 4.52-4.44 (m, |
| | yl)difluoro- | | | 2H), 4.43-4.38 |
| | methyl) | | | (m, 1H), 2.32- |
| | phosphonic | | | 2.17 (m, 2H), |
| | acid | | | 2.13-2.05 (m, |
| | | | | 1H), 2.02-1.91 |
| | | | | (m, 5H), 1.85- |
| | | | | 1.79 (m, 1H), 1.75 |
| | | | | (d, J = 10.0 Hz, |
| | | | | 1H), 1.70- |
| | | | | 1.53 (m, 2H) |
| 1113 | ((2- (((3S,6S,10aS)- 3-((1-(1H- pyrrolo[2,3- b]pyridin-3- yl)ethyl)car- bamoyl)-5- oxodecahydro pyrrolo[1,2- a]azocin-6- yl)carbamoyl) | 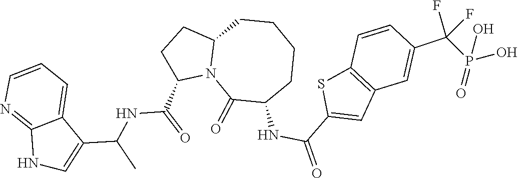 [SMILES: CC(NC(=O)[C@@H]1CC[C@@H]2CCCC[C@H](NC(=O)c3cc4cc(C(F)(F)P(=O)(O)O)ccc4s3)C(=O)N21)c1c[nH]c2ncccc12] | 660.2 | ¹H NMR (400 MHz, CD₃OD) δ 8.46 (d, J = 8.4 Hz, 1H), 8.33 (d, J = 7.2 Hz, 1H), 8.14 (s, 1H), 8.04 (s, 1H), 7.86 (d, J = 8.4 Hz, 1H), 7.70 (d, J = 8.4 Hz, 1H), 7.45 (s, |
| | benzo[b]thio- | | | 1H), 7.18-7.10 |
| | phen-5- | | | (m, 1H), 5.37- |
| | yl)difluoro- | | | 5.27 (m, 1H), |
| | methyl) | | | 5.01 (s, 1H), |
| | phosphonic | | | 4.48-4.36 (m, |
| | acid | | | 2H), 2.29-2.16 |
| | | | | (m, 2H), 2.15- |
| | | | | 2.05 (m, 1H), |
| | | | | 2.05-1.86 (m, |
| | | | | 5H), 1.85-1.61 |
| | | | | (m, 4H), 1.59 (d, |
| | | | | J = 6.8 Hz, 3H) |
| 1114 | ((2- (((3S,6S,10aS)- 3-(((1H- pyrrolo[2,3- b]pyridin-5- yl)methyl)car- bamoyl)-5- oxodecahydro pyrrolo[1,2- a]azocin-6- yl)carbamoyl) | 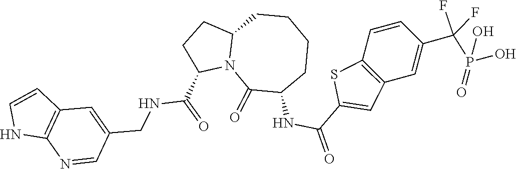 [SMILES: O=C(N[C@H]1CCCC[C@H]2CC[C@@H](C(=O)NCc3cnc4[nH]ccc4c3)N2C1=O)c1cc2cc(C(F)(F)P(=O)(O)O)ccc2s1] | 646.2 | ¹H NMR (400 MHz, CD₃OD) δ 8.21-8.13 (m, 2H), 8.08 (s, 1H), 8.01 (d, J = 2.0 Hz, 1H), 7.88 (d, J = 8.4 Hz, 1H), 7.72 (d, J = 8.4 Hz, 1H), 7.37 (d, J = 3.6 Hz, 1H), |
| | benzo[b]thio- | | | 6.47 (d, J = 3.2 Hz, |
| | phen-5- | | | 1H), 5.08- |
| | yl)difluoro- | | | 5.00 (m, 1H), |
| | methyl) | | | 4.58-4.51 (m, |
| | phosphonic | | | 1H), 4.50-4.43 |
| | acid | | | (m, 2H), 4.43- |
| | | | | 4.32 (m, 1H), |
| | | | | 2.30-2.17 (m, |
| | | | | 2H), 2.14-2.05 |
| | | | | (m, 1H), 2.04- |
| | | | | 1.90 (m, 5H), |
| | | | | 1.85-1.73 (m, |
| | | | | 2H), 1.71-1.56 |
| | | | | (m, 2H) |
| 1115 | ((2- (((3S,6S,9aS)- 3-(((1H- benzo[d] imidazol-5- yl)methyl)car- bamoyl)-5- oxooctahydro- 1H- pyrrolo[1,2- a]azepin-6- yl)carbamoyl) benzo[b]thio- | 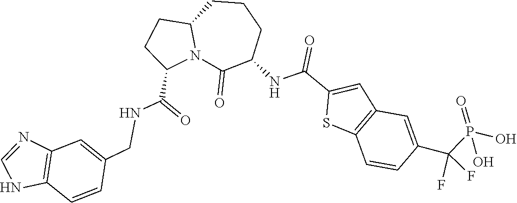 | 632.1 | ¹H NMR (400 MHz, CD₃OD) δ 8.62 (s, 1H), 8.14 (s, 1H), 7.96 (s, 1H), 7.88 (d, J = 8.4 Hz, 1H), 7.75- 7.70 (m, 1H), 7.65 (s, 1H), 7.54 (d, J = 8.4 Hz, 1H), 7.37 (d, J = 8.4 Hz, 1H), 4.74 (d, J = |
| | phen-5- | | | 10.4 Hz, 1H), |
| | yl)difluoro- | | | 4.70-4.56 (m, |
| | methyl) | | | 2H), 4.44 (d, J = |
| | phosphonic | | | 15.2 Hz, 1H), |
| | acid | | | 4.12-4.02 (m, |
| | | | | 1H), 2.36-2.25 |
| | | | | (m, 1H), 2.22- |
| | | | | 2.11 (m, 1H), |
| | | | | 2.10-1.98 (m, |
| | | | | 3H), 1.96-1.76 |
| | | | | (m, 5H) |
| 1116 | (difluoro(2- (((3S,6S,9aS)- 3-(((2-methyl- 1H- benzo[d] imidazol-5- yl)methyl)car- bamoyl)-5- oxooctahydro- 1H- pyrrolo[1,2- a]azepin-6- | 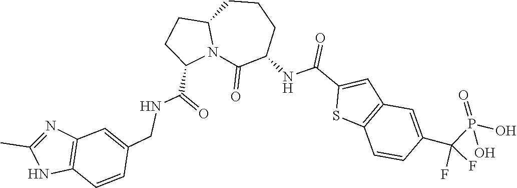 | 646.2 | ¹H NMR (400 MHz, CD₃OD) δ 8.12 (s, 1H), 7.90-7.85 (m, 2H), 7.72 (d, J = 8.4 Hz, 1H), 7.51 (s, 1H), 7.42-7.37 (m, 1H), 7.30 (d, J = 8.4 Hz, 1H), 4.73 (d, J = 10.4 Hz, 1H), 4.65- |
| | yl)carbamoyl) | | | 4.56 (m, 2H), 4.42 |
| | benzo[b]thio- | | | (d, J = 15.2 Hz, |
| | phen-5- | | | 1H), 4.11- |
| | yl)methyl) | | | 4.02 (m, 1H), |
| | phosphonic | | | 2.59 (s, 3H), |
| | acid | | | 2.36-2.26 (m, |
| | | | | 1H), 2.21-2.12 |
| | | | | (m, 1H), 2.11- |
| | | | | 1.99 (m, 3H), |
| | | | | 1.95-1.80 (m, |
| | | | | 5H) |
| 1117 | (difluoro(2- (((3S,6S,9aS)- 5-oxo-3-(((2- oxo-2,3- dihydro-1H- benzo[d] imidazol-5- yl)methyl)car- bamoyl)octa- hydro-1H- pyrrolo[1,2- a]azepin-6- | 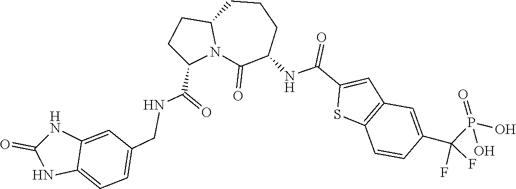 | 648.1 | ¹H NMR (400 MHz, CD₃OD) δ 8.19 (s, 1H) 8.09 (s, 1H) 7.92 (s, 1H) 7.72- 7.78 (m, 1H) 7.03 (s, 1H) 6.96- 7.00 (m, 2H) 4.74 (m, 1H) 4.61 (m, 1H) 4.43-4.49 (m, 1H) 4.36 (s, 1H) |
| | yl)carbamoyl) | | | 4.00-4.08 (m, |
| | benzo[b]thio- | | | 1H) 2.23-2.35 |
| | phen-5- | | | (m, 1H) 2.04 (s, |
| | yl)methyl) | | | 4H) 1.86 (m, |
| | phosphonic | | | 5H) |
| | acid | | | |
| 1118 | ((2- (((3S,6S,9aS)- 3-(((3H- imidazo[4,5- b]pyridin-6- yl)methyl)car- bamoyl)-5- oxooctahydro- 1H- pyrrolo[1,2- a]azepin-6- yl)carbamoyl) benzo[b]thio- phen-5- | 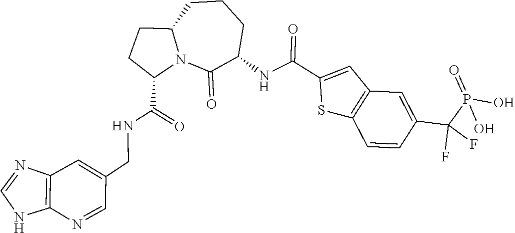 | 633.2 | ¹H NMR (400 MHz, DMSO- d₆) δ 8.77 (d, J = 7.2 Hz, 1H), 8.51 (t, J = 5.6 Hz, 1H), 8.38 (s, 1H), 8.28 (s, 1H), 8.23 (s, 1H), 8.05 (s, 1H), 7.96 (d, J = 8.4 Hz, 1H), 7.87 (s, 1H), 7.62 (d, J = 8.4 Hz, 1H), 4.62 |
| | yl)difluoro- | | | (d, J = 7.2 Hz, |
| | methyl) | | | 1H), 4.50-4.45 |
| | phosphonic | | | (m, 2H), 4.42- |
| | acid | | | 4.32 (m, 1H), |
| | | | | 3.96 (d, J = |
| | | | | 6.4 Hz, 1H), 2.25- |
| | | | | 2.13 (m, 1H), |
| | | | | 2.02-1.91 (m, |
| | | | | 2H), 1.90-1.81 |
| | | | | (m, 2H), 1.73 (s, |
| | | | | 5H) |
| 1119 | (difluoro(2- (((3S,6S,9aS)- 3-(((3-methyl- 3H- imidazo[4,5- b]pyridin-6- yl)methyl)car- bamoyl)-5- oxooctahydro- 1H- pyrrolo[1,2- a]azepin-6- yl)carbamoyl) | 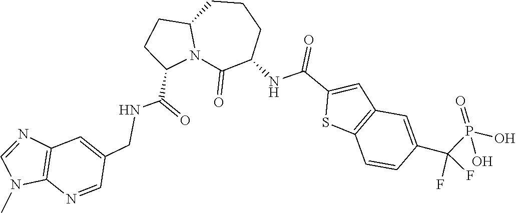 | 647.2 | ¹H NMR (400 MHz, DMSO- d₆) δ 8.77 (d, J = 7.2 Hz, 1H), 8.53 (t, J = 6.0 Hz, 1H), 8.41 (s, 1H), 8.30 (d, J = 1.6 Hz, 1H), 8.25 (s, 1H), 8.05 (s, 1H), 8.00 (d, J = 8.4 Hz, 1H), 7.94 (d, J = 1.6 Hz, |
| | benzo[b]thio- | | | 1H), 7.61 (d, J = |
| | phen-5- | | | 8.8 Hz, 1H), |
| | yl)methyl) | | | 7.39-7.17 (m, |
| | phosphonic | | | 2H), 4.64 (t, J = |
| | acid | | | 8.4 Hz, 1H), |
| | | | | 4.53-4.44 (m, |
| | | | | 2H), 4.42-4.34 |
| | | | | (m, 1H), 4.00- |
| | | | | 3.93 (m, 1H), |
| | | | | 3.82 (s, 3H), |
| | | | | 2.23-2.15 (m, |
| | | | | 1H), 2.05-1.92 |
| | | | | (m, 2H), 1.91- |
| | | | | 1.81 (m, 2H), |
| | | | | 1.80-1.66 (m, |
| | | | | 5H) |
| 1120 | (difluoro(2- (((3S,6S,9aS)- 3-(((1-methyl- 1H- imidazo[4,5- b]pyridin-6- yl)methyl)car- bamoyl)-5- oxooctahydro- 1H- pyrrolo[1,2- a]azepin-6- | 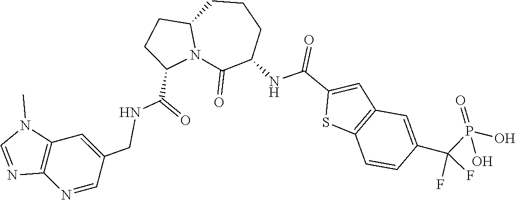 | 647.1 | ¹H NMR (400 MHz, DMSO- d₆) δ 8.74 (d, J = 7.2 Hz, 1H), 8.57 (t, J = 6.0 Hz, 1H), 8.44 (s, 1H), 8.36 (s, 1H), 8.29 (s, 1H), 8.15-8.08 (m, 2H), 8.04 (s, 1H), 7.59 (d, J = 8.4 Hz, 1H), |
| | yl)carbamoyl) | | | 4.69-4.53 (m, |
| | benzo[b]thio- | | | 3H), 4.48 (m, |
| | phen-5- | | | 1H), 4.39-4.33 |
| | yl)methyl) | | | (m, 1H), 4.00 (d, |
| | phosphonic | | | J = 4.8 Hz, 1H), |
| | acid | | | 3.79 (s, 3H), |
| | | | | 2.24-2.16 (m, |
| | | | | 1H), 2.07-1.94 |
| | | | | (m, 2H), 1.91- |
| | | | | 1.84 (m, 2H), |
| | | | | 1.77 (s, 1H), |
| | | | | 1.76-1.70 (m, |
| | | | | 4H) |
| 1121 | (difluoro(2- (((3S,6S,10aS)- 3-((2- methoxy-1- (1H- pyrrolo [2,3- b]pyridin-3- yl)ethyl)car- bamoyl)-5- oxodecahydro pyrrolo[1,2- a]azocin-6- yl)carbamoyl) benzo[b]thio- phen-5- | 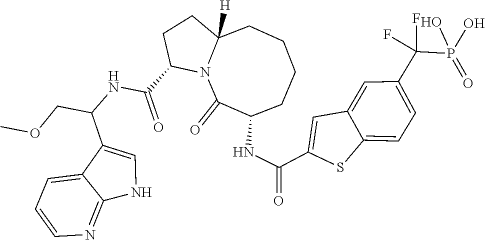 [SMILES: COCC(NC(=O)[C@@H]1CC[C@@H]2CCCC[C@H](NC(=O)c3cc4cc(C(F)(F)P(=O)(O)O)ccc4s3)C(=O)N21)c1c[nH]c2ncccc12] | 690.2 | ¹H NMR (400 MHz, CD₃OD) δ 8.50-8.20 (m, 1H), 8.19-8.07 (m, 2H), 7.98 (s, 2H), 7.73-7.62 (m, 1H), 7.52- 7.44 (m, 1H), 7.38-7.23 (m, 1H), 5.44 (d, J = 5.6 Hz, 1H), 5.00-4.94 (m, 1H), 4.50-4.37 (m, 2H), 3.94- 3.75 (m, 2H), |
| | yl)methyl) | | | 3.43-3.38 (m, |
| | phosphonic | | | 3H), 2.31-2.09 |
| | acid | | | (m, 3H), 2.07- |
| | | | | 1.87 (m, 6H), |
| | | | | 1.82-1.62 (m, |
| | | | | 3H) |
| 1122 | ((2-(((35,6S)- 3-((3-(2- amino-2- oxoethyl)- [1,1′- biphenyl]-4- yl)carbamoyl)- 5- oxooctahydro- 1H- pyrrolo[1,2- a]azepin-6- yl)carbamoyl) benzo[b]thio- phen-5- yl)difluoro- methyl) phosphonic acid | 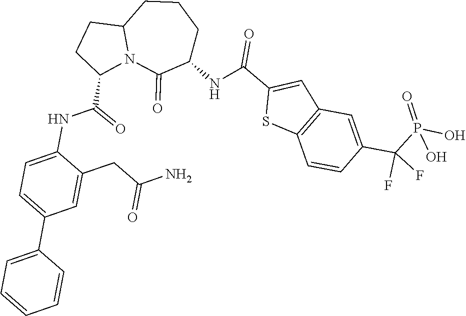 [SMILES: NC(=O)Cc1cc(-c2ccccc2)ccc1NC(=O)[C@@H]1CCC2CCC[C@H](NC(=O)c3cc4cc(C(F)(F)P(=O)(O)O)ccc4s3)C(=O)N21] | 711.4 | ¹H NMR (400 MHz, CD₃OD) δ 8.21-8.13 (m, 2H), 8.04 (d, J = 8.4 Hz, 1H), 7.70 (d, J = 8.4 Hz, 1H), 7.67- 7.60 (m, 4H), 7.59-7.55 (m, 1H), 7.45 (t, J = 7.6 Hz, 2H), 7.38-7.31 (m, 1H), 4.86-4.80 (m, 1H), 4.78 (dd, J = 4.8, 7.6 Hz, 1H), 4.19- 4.02 (m, 1H), 3.77-3.59 (m, 2H), 2.45-2.32 |
| | | | | (m, 1H), 2.30- |
| | | | | 2.21 (m, 2H), |
| | | | | 2.15-2.08 (m, |
| | | | | 1H), 2.07-1.90 |
| | | | | (m, 5H), 1.89- |
| | | | | 1.80 (m, 1H). |
| 1123 | | 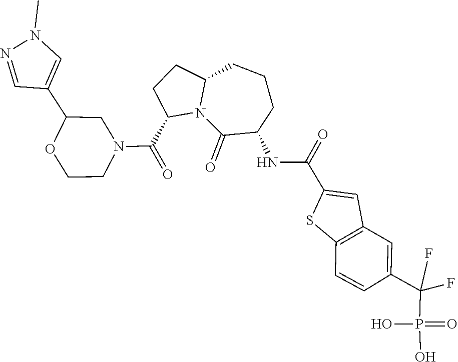 [SMILES: Cn1cc(C2CN(C(=O)[C@@H]3CC[C@@H]4CCC[C@H](NC(=O)c5cc6cc(C(F)(F)P(=O)(O)O)ccc6s5)C(=O)N43)CCO2)cn1] | 652.0 | ¹H NMR (400 MHz, DMSO- d₆) δ 8.82-8.67 (m, 1H), 8.29 (d, J = 6.1 Hz, 1H), 8.13 (d, J = 7.7 Hz, 1H), 8.07 (s, 1H), 7.80-7.65 (m, 1H), 7.61- 7.55 (m, 1H), 7.46-7.38 (m, 1H), 5.07-4.89 (m, 1H), 4.68- 4.59 (m, 1H), 4.45-4.19 (m, 2H), 4.12-3.82 (m, 4H), 3.82- 3.75 (m, 3H), 3.62-3.44 (m, 2H), 3.32-3.25 (m, 1H), 2.88- 2.75 (m, 1H), |
| | | | | 2.26-1.62 (m, |
| | | | | 10H) |
| 1124 | (difluoro(2- (((3S,6S,9aS)- 5-oxo-3-(2- (pyridin-3- yl)morpholine- 4- carbonyl)octa- hydro-1H- pyrrolo[1,2- a]azepin-6- yl)carbamoyl) benzo[b]thio- phen-5- yl)methyl) phosphonic acid | 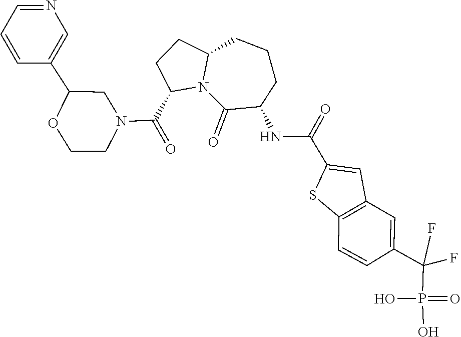 [SMILES: O=C(N[C@H]1CCC[C@H]2CC[C@@H](C(=O)N3CCOC(c4cccnc4)C3)N2C1=O)c1cc2cc(C(F)(F)P(=O)(O)O)ccc2s1] | 649 | ¹H NMR (400 MHz, DMSO- d₆) δ 8.83-8.69 (m, 1H), 8.67- 8.59 (m, 1H), 8.58-8.51 (m, 1H), 8.33-8.25 (m, 1H), 8.17- 8.10 (m, 1H), 8.09-8.04 (m, 1H), 7.90-7.80 (m, 1H), 7.61- 7.55 (m, 1H), 7.46-7.40 (m, 1H), 5.17-4.89 (m, 1H), 4.69- 4.57 (m, 1H), 4.54-4.34 (m, 1H), 4.28-4.12 (m, 1H), 4.05- 3.98 (m, 2H), |
| | | | | 3.64-3.55 (m, |
| | | | | 1H), 3.38-3.31 |
| | | | | (m, 1H), 3.21- |
| | | | | 3.13 (m, 1H), |
| | | | | 2.85-2.66 (m, |
| | | | | 1H), 2.30-1.94 |
| | | | | (m, 3H), 1.93- |
| | | | | 1.60 (m, 7H) |
| 1125 | (difluoro(2- (((3S,6S,9aS)- 5-oxo-3-(7- (pyridin-3-yl)- 4,7- diazaspiro[2.5] octane-4- carbonyl)octa- hydro-1H- pyrrolo[1,2- a]azepin-6- yl)carbamoyl) benzo[b]thio- phen-5- yl)methyl) phosphonic acid | 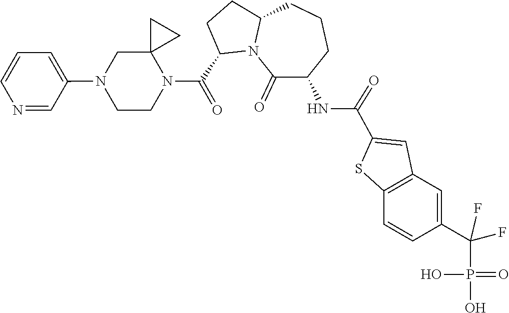 [SMILES: O=C(N[C@H]1CCC[C@H]2CC[C@@H](C(=O)N3CCN(c4cccnc4)CC34CC4)N2C1=O)c1cc2cc(C(F)(F)P(=O)(O)O)ccc2s1] | 674 | ¹H NMR (400 MHz, DMSO- d₆) δ 8.67 (d, J = 6.8 Hz, 1H), 8.25 (s, 1H), 8.10-8.03 (m 1H), 8.00-7.75 (m, 3H), 7.73- 7.63 (m, 1H), 7.56-7.41 (m, 2H), 5.10 (s, 1H), 4.69-4.65 (m, 1H), 4.09- 4.02 (m, 2H), 3.80-3.74 (m, 1H), 3.62-3.56 (m, 1H), 3.15- 3.10 (m, 1H), 2.28-2.13 (m, |
| | | | | 2H), 2.04-1.64 |
| | | | | (m, 10H), 1.30- |
| | | | | 1.18 (m, 2H), |
| | | | | 0.97-0.79 (m, |
| | | | | 2H) |
| 1126 | (difluoro(2- (((3S,6S,9aS)- 3-(2-(4- methyl-4H- 1,2,4-triazol- 3- yl)morpholine- 4-carbonyl)- 5- oxooctahydro- 1H- pyrrolo[1,2- a]azepin-6- yl)carbamoyl) benzo[b]thio- phen-5- yl)methyl) phosphonic acid | 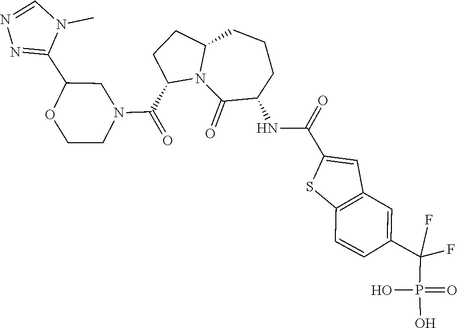 [SMILES: Cn1cnnc1C1CN(C(=O)[C@@H]2CC[C@@H]3CCC[C@H](NC(=O)c4cc5cc(C(F)(F)P(=O)(O)O)ccc5s4)C(=O)N32)CCO1] | 653 | ¹H NMR (400 MHz, DMSO- d₆) δ 8.85-8.62 (m, 2H), 8.30 (s, 1H), 8.14 (d, J = 8.5 Hz, 1H), 8.08 (s, 1H), 7.59 (d, J = 8.5 Hz, 1H), 5.09- 4.88 (m, 1H), 4.85-4.60 (m, 2H), 4.58-3.77 (m, 4H), 3.75- 3.56 (m, 4H), 3.49-3.24 (m, 1H), 3.21-2.74 (m, 1H), 2.27- 1.62 (m, 10H) |
| 1127 | (difluoro(2- (((3S,6S,9aS)- 3-(2-(5- methyl-1,3,4- oxadiazol-2- yl)morpholine- 4-carbonyl)- 5- oxooctahydro- 1H- pyrrolo[1,2- a]azepin-6- yl)carbamoyl) benzo[b]thio- phen-5- yl)methyl) phosphonic acid | 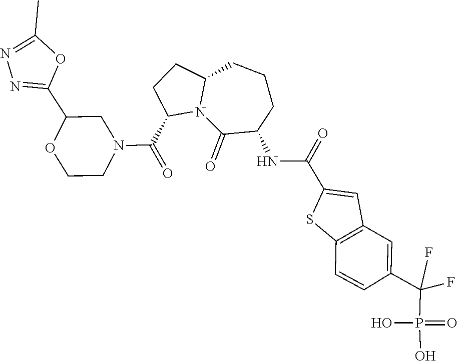 [SMILES: Cc1nnc(C2CN(C(=O)[C@@H]3CC[C@@H]4CCC[C@H](NC(=O)c5cc6cc(C(F)(F)P(=O)(O)O)ccc6s5)C(=O)N43)CCO2)o1] | 654 | ¹H NMR (400 MHz, DMSO- d₆) δ 8.83-8.68 (m, 1H), 8.30 (s, 1H), 8.17-8.05 (m, 2H), 7.59 (d, J = 8.3 Hz, 1H), 5.09-4.77 (m, 2H), 4.75-4.58 (m, 1H), 4.50- 4.16 (m, 1H), 4.06-3.85 (m, 3H), 3.79-3.02 (m, 3H), 2.48 (s, 3H), 2.28-2.16 (m, 1H), 2.12- 1.68 (m, 9H) |

### Biochemical and Cellular Assays

### STAT3 Fluorescence Polarization (FP) Assay

An FP assay was developed to determine IC₅₀ values for test substances. Recombinant STAT3 protein (STAT3 (G127-I722)) at 25 nM was combined with a fluorescently labeled, phosphotyrosine peptide probe (5-FAM-GpYLPQTV-NH₂) at 2 nM in FP buffer (10 mM HEPES pH 7.4, 50 mM NaCl, 1 mM EDTA, 0.05% Tween 20.2 mM DTT). 50 μL of STAT3-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 112. For STAT3 FP assay, A=≤100 nM; B=>100-1000 nM; C=>1-10 μM; and D=>10 μM.

### STAT6 Fluorescence Polarization (FP) Assay

An FP assay was developed to determine IC₅₀ values for test substances. Recombinant STAT6 protein (STAT6 (W123-T658)) at 250 nM was combined with a fluorescently labeled, phosphotyrosine peptide probe (5-FAM-ApYKPFQDLI-NH₂) at 2 nM in FP buffer (10 mM HEPES pH 7.4, 50 mM NaCl, 1 mM EDTA, 0.05% Tween 20, 2 mM DTT). 50 μL of STAT6-probe mixture was added to serial diluted compounds in black, 96-well plates (Greiner BioOne 655076) to a final concentration of 1% DMSO. Reaction components were mixed, and FP was measured after 45-minute incubation at room temperature using a Tecan Spark multimode plate reader. FP signal (mP) was plotted against the log concentration of the test substances and IC₅₀ values were calculated by nonlinear regression analysis using GraphPad Prism software. Results are shown in Table 112. For STAT6 FP assay, A=≤300 nM; B=>300-3000 nM; C=>3-30 μM; and D=>30 μM.

TABLE 112

| Cmp# | STAT3 | STAT6 |
|---|---|---|
| 1 | D | B |
| 2 | B | C |
| 3 | B | C |
| 4 | B | C |
| 5 | B | C |
| 6 | B | C |
| 7 | B | C |
| 8 | B | C |
| 9 | B | C |
| 10 | B | C |
| 11 | B | C |
| 12 | C | A |
| 13 | A | C |
| 14 | B | A |
| 15 | A | B |
| 16 | A | B |
| 17 | A | C |
| 18 | C | A |
| 19 | C | A |
| 20 | B | C |
| 21 | C | A |
| 22 | C | A |
| 23 | C | B |
| 24 | B | A |
| 25 | A | B |
| 26 | A | B |
| 27 | C | A |
| 28 | C | A |
| 29 | B | C |
| 30 | C | B |
| 31 | B | B |
| 32 | A | C |
| 33 | A | B |
| 34 | B | C |
| 35 | D | C |
| 36 | A | B |
| 37 | B | B |
| 38 | A | B |
| 39 | B | B |
| 40 | B | C |
| 41 | A | A |
| 42 | B | A |
| 43 | A | B |
| 44 | C | B |
| 45 | A | B |
| 46 | A | C |
| 47 | C | B |
| 48 | C | C |
| 49 | B | C |
| 50 | B | B |
| 51 | A | B |
| 52 | C | B |
| 53 | A | B |
| 54 | A | B |
| 55 | B | B |
| 56 | B | C |
| 57 | B | B |
| 58 | B | B |
| 59 | B | B |
| 60 | A | C |
| 61 | B | B |
| 62 | D | D |
| 63 | B | A |
| 64 | A | B |
| 65 | B | B |
| 66 | A | C |
| 67 | A | B |
| 68 | B | C |
| 69 | B | B |
| 70 | B | C |
| 71 | B | B |
| 72 | B | B |
| 73 | B | C |
| 74 | C | A |
| 75 | C | A |
| 76 | C | A |
| 77 | B | C |
| 78 | B | D |
| 79 | B | A |
| 80 | B | A |
| 81 | A | B |
| 82 | B | A |
| 83 | B | A |
| 84 | B | B |
| 85 | A | C |
| 86 | B | A |
| 87 | B | B |
| 88 | C | B |
| 89 | C | A |
| 90 | D | B |
| 91 | C | A |
| 92 | B | B |
| 93 | B | C |
| 94 | B | C |
| 95 | B | A |
| 96 | B | A |
| 97 | B | B |
| 98 | B | C |
| 99 | B | B |
| 100 | C | A |
| 101 | B | B |
| 102 | B | B |
| 103 | A | C |
| 104 | B | B |
| 105 | B | A |
| 106 | C | A |
| 107 | B | A |
| 108 | A | B |
| 109 | A | C |
| 110 | A | C |
| 111 | C | B |
| 113 | B | C |
| 114 | A | C |
| 115 | A | C |
| 116 | B | B |
| 117 | A | C |
| 118A | B | C |
| 118B | B | B |
| 119 | B | C |
| 120 | B | B |
| 121 | B | B |
| 122 | A | C |
| 123 | A | C |
| 124 | A | C |

TABLE 113

| Cmp# | STAT3 | STAT6 |
|---|---|---|
| 203 | B | B |
| 205 | A | C |
| 206 | A | C |
| 207 | A | C |
| 208 | C | C |
| 209 | A | B |
| 210 | A | C |
| 211 | A | C |
| 212 | C | A |
| 213 | A | C |
| 214 | B | C |
| 215 | C | C |
| 216 | B | C |
| 217 | C | C |
| 218 | B | A |
| 219 | C | C |
| 220 | C | C |
| 221 | A | C |
| 222 | B | B |
| 223 | B | B |
| 224 | C | A |
| 225 | C | A |
| 226 | C | A |
| 227 | B | B |
| 228 | C | B |
| 229 | C | A |
| 230 | C | A |
| 231 | C | A |
| 232 | C | B |
| 233 | A | C |
| 234 | C | A |
| 235 | C | A |
| 236 | A | C |
| 237 | C | A |
| 238 | C | A |
| 239 | C | A |
| 240 | C | A |
| 241 | A | C |
| 244 | C | B |
| 245 | A | C |
| 246 | A | C |
| 247 | A | C |
| 495 | B | C |
| 496 | A | B |
| 497 | B | B |
| 498 | A | B |
| 499 | A | B |
| 500 | B | C |
| 501 | B | C |
| 502 | B | C |
| 503 | B | C |
| 504 | C | C |
| 505 | C | C |
| 506 | C | C |
| 507 | C | C |
| 508 | C | C |
| 509 | C | C |
| 510 | C | C |
| 511 | C | C |
| 512 | A | C |
| 513 | B | B |
| 514 | A | C |
| 515 | C | C |
| 516 | A | B |
| 517 | B | C |
| 518 | B | C |
| 519 | C | C |
| 520 | C | C |
| 521 | A | C |
| 522 | C | B |
| 523 | C | C |
| 524 | B | B |
| 525 | C | B |
| 526 | A | C |
| 527 | C | B |
| 528 | C | B |
| 529 | B | C |
| 530 | B | C |
| 531 | C | C |
| 532 | A | C |
| 533 | C | B |
| 534 | B | C |
| 535 | C | C |
| 536 | C | C |
| 537 | C | C |
| 538 | C | C |
| 539 | A | C |
| 540 | A | B |
| 541 | C | C |
| 542 | C | C |
| 543 | C | C |
| 544 | C | C |
| 545 | C | C |
| 546 | C | C |
| 547 | A | B |
| 548 | C | C |
| 549 | A | C |
| 550 | B | C |
| 551 | C | A |
| 552 | C | A |
| 553 | C | B |
| 554 | C | B |
| 555 | B | C |
| 556 | C | C |
| 557 | C | C |
| 558 | A | B |
| 559 | C | B |
| 560 | C | C |
| 561 | C | C |
| 562 | B | C |
| 563 | C | B |
| 564 | B | C |
| 565 | B | C |
| 566 | A | C |
| 567 | B | C |
| 568 | A | C |
| 569 | C | B |
| 570 | C | C |
| 571 | C | C |
| 572 | C | B |
| 573 | B | C |
| 574 | C | C |
| 575 | C | C |
| 576 | B | C |
| 577 | C | C |
| 578 | C | C |
| 579 | B | C |
| 580 | C | C |
| 581 | C | B |
| 582 | C | C |
| 583 | B | C |
| 584 | C | C |
| 585 | A | C |
| 586 | C | B |
| 587 | C | C |
| 588 | C | C |
| 589 | C | C |
| 590 | C | C |
| 591 | C | C |
| 592 | B | C |
| 593 | C | C |
| 594 | C | C |
| 595 | B | C |
| 596 | B | C |
| 597 | A | C |
| 598 | A | C |
| 599 | A | B |
| 600 | A | B |
| 601 | C | A |
| 602 | A | C |
| 603 | A | C |
| 604 | B | C |
| 605 | A | C |
| 606 | A | C |
| 607 | A | B |
| 608 | A | B |
| 609 | B | C |
| 610 | B | C |
| 611 | B | C |
| 612 | A | C |
| 613 | B | C |
| 614 | B | C |
| 615 | A | C |
| 616 | A | C |
| 617 | B | C |
| 618 | C | C |
| 619 | B | C |
| 620 | B | B |
| 621 | A | C |
| 622 | A | C |
| 623 | B | C |
| 624 | C | C |
| 625 | B | C |
| 626 | A | C |
| 627 | A | C |
| 628 | A | C |
| 629 | B | C |
| 630 | B | C |
| 631 | A | C |
| 632 | C | C |
| 633 | B | C |
| 634 | A | B |
| 635 | B | C |
| 636 | A | C |
| 637 | A | B |
| 638 | A | C |
| 639 | A | C |
| 640 | B | C |
| 641 | A | B |
| 642 | B | C |
| 643 | A | B |
| 644 | B | C |
| 645 | B | C |
| 646 | C | C |
| 647 | B | C |
| 648 | A | C |
| 649 | C | C |
| 650 | C | C |
| 651 | C | C |
| 652 | B | B |
| 653 | B | C |
| 654 | B | C |
| 655 | B | C |
| 656 | B | C |
| 657 | C | C |
| 658 | B | B |
| 659 | C | C |
| 660 | C | C |
| 661 | C | C |
| 662 | C | C |
| 663 | B | C |
| 664 | A | B |
| 665 | A | C |
| 666 | A | C |
| 667 | A | C |
| 668 | A | C |
| 669 | A | C |
| 670 | B | C |
| 671 | A | B |
| 672 | C | C |
| 673 | B | C |
| 674 | B | C |
| 675 | A | C |
| 676 | A | C |
| 677 | A | C |
| 678 | A | C |
| 679 | A | C |
| 680 | A | C |
| 681 | A | C |
| 682 | B | C |
| 683 | A | C |
| 684 | A | C |
| 685 | A | C |
| 686 | A | C |
| 687 | A | C |
| 688 | A | C |
| 689 | A | B |
| 690 | A | C |
| 691 | A | C |
| 692 | A | C |
| 693 | C | C |
| 694 | A | B |
| 695 | A | C |
| 696 | C | C |
| 697 | C | C |
| 698 | B | C |
| 699 | A | C |
| 700 | B | C |
| 701 | B | C |
| 702 | B | C |
| 703 | A | C |
| 704 | A | C |
| 705 | A | C |
| 706 | A | B |
| 707 | A | B |
| 708 | B | C |
| 709 | B | C |
| 710 | B | C |
| 711 | C | C |
| 712 | C | C |
| 713 | B | C |
| 714 | B | C |
| 715 | B | C |
| 716 | C | C |
| 717 | A | C |
| 718 | A | B |
| 719 | B | C |
| 720 | C | C |
| 721 | B | C |
| 722 | B | C |
| 723 | B | C |
| 724 | A | B |
| 725 | B | C |
| 726 | B | C |
| 727 | C | C |
| 728 | B | C |
| 729 | B | A |
| 730 | C | C |
| 731 | C | C |
| 732 | C | C |
| 733 | C | B |
| 734 | B | B |
| 735 | A | C |
| 736 | A | C |
| 737 | A | C |
| 738 | A | B |
| 739 | A | C |
| 740 | A | C |
| 741 | A | B |
| 742 | A | B |
| 743 | C | C |
| 744 | A | C |
| 745 | A | C |
| 746 | A | C |
| 747 | A | C |
| 748 | A | C |
| 749 | A | C |
| 750 | A | C |
| 751 | B | C |
| 752 | B | C |
| 753 | A | C |
| 754 | C | C |
| 755 | A | C |
| 756 | A | C |
| 757 | B | C |
| 758 | C | B |
| 759 | D | B |
| 760 | C | C |
| 761 | A | C |
| 762 | A | B |
| 763 | C | C |
| 764 | B | C |
| 765 | A | C |
| 766 | A | C |
| 767 | A | C |
| 768 | B | C |
| 769 | A | C |
| 770 | C | C |
| 771 | A | C |
| 772 | A | C |
| 773 | B | D |
| 774 | A | C |
| 775 | A | B |
| 776 | A | B |
| 777 | A | B |
| 778 | A | B |
| 779 | A | C |
| 780 | B | B |
| 781 | B | A |
| 782 | B | B |
| 783 | D | D |
| 784 | B | C |
| 785 | B | C |
| 786 | C | B |
| 787 | A | B |
| 788 | C | B |
| 789 | B | B |
| 790 | A | C |
| 791 | B | B |
| 792 | B | B |
| 793 | C | C |
| 794 | B | C |
| 795 | B | B |
| 796 | B | C |
| 513 | B | B |
| 797 | B | C |
| 798 | C | B |
| 799 | A | B |
| 800 | C | B |
| 801 | B | C |
| 802 | C | B |
| 803 | A | B |
| 804 | B | B |
| 805 | B | C |
| 806 | A | C |
| 807 | B | B |
| 808 | B | C |
| 809 | A | C |
| 810 | B | B |
| 811 | A | B |
| 812 | B | C |
| 813 | C | B |
| 814 | C | B |
| 815 | C | B |
| 816 | B | C |
| 817 | C | C |
| 818 | B | C |
| 819 | B | B |
| 820 | B | C |
| 821 | B | B |
| 822 | B | C |
| 823 | B | C |
| 824 | A | B |
| 825 | B | B |
| 826 | B | B |
| 827 | B | C |
| 828 | A | B |
| 829 | C | B |
| 830 | B | B |
| 831 | C | B |
| 832 | C | A |
| 833 | C | A |
| 834 | B | A |
| 835 | C | A |
| 836 | C | A |
| 837 | C | A |
| 838 | C | A |
| 839 | C | A |
| 840 | C | A |
| 841 | C | B |
| 842 | C | B |
| 843 | C | B |
| 844 | C | B |
| 845 | C | A |
| 846 | C | B |
| 847 | C | B |
| 848 | C | B |
| 849 | C | A |
| 850 | C | C |
| 851 | B | C |
| 852 | A | B |
| 853 | C | C |
| 854 | C | C |
| 855 | A | C |
| 856 | A | B |
| 857 | A | C |
| 858 | A | B |
| 859 | A | B |
| 860 | B | C |
| 861 | A | C |
| 862 | A | C |
| 863 | A | C |
| 864 | D | C |
| 865 | C | A |
| 866 | B | A |
| 867 | B | A |
| 870 | A | C |
| 871 | C | B |
| 872 | B | A |
| 873 | A | C |
| 874 | C | B |
| 875 | B | A |
| 876 | C | B |
| 877 | C | A |
| 878 | C | B |
| 879 | C | A |
| 880 | C | A |
| 882 | C | B |
| 883 | C | B |
| 884 | C | B |
| 885 | C | B |
| 886 | C | B |
| 887 | C | B |
| 888 | C | B |
| 889 | C | A |
| 890 | C | A |
| 891 | C | A |
| 892 | C | B |
| 893 | C | B |
| 894 | C | A |
| 895 | C | B |
| 896 | B | C |
| 897 | C | C |
| 898 | C | A |
| 899 | C | B |
| 900 | C | A |
| 901 | C | C |
| 902 | C | C |
| 903 | C | B |
| 904 | C | B |
| 905 | B | B |
| 906 | C | B |
| 907 | C | A |
| 908 | C | A |
| 909 | C | A |
| 910 | C | A |
| 911 | C | A |
| 912 | C | A |
| 913 | C | B |
| 914 | B | B |
| 915 | C | A |
| 916 | C | A |
| 917 | C | A |
| 918 | A | C |
| 919 | C | A |
| 920 | C | A |
| 921 | C | A |
| 922 | C | B |
| 923 | C | A |
| 924 | C | A |
| 925 | A | C |
| 926 | A | C |
| 927 | A | C |
| 928 | C | C |
| 929 | C | A |
| 930 | B | C |
| 931 | A | B |
| 932 | A | C |
| 933 | A | C |
| 934 | C | A |
| 935 | C | A |
| 936 | C | A |
| 937 | C | A |
| 938 | A | B |
| 939 | A | C |
| 1107 | B | B |
| 1108 | B | C |
| 1109 | B | B |
| 1110 | A | B |
| 1111 | C | C |
| 1112 | A | B |
| 1113 | B | B |
| 1114 | B | B |
| 1115 | A | B |
| 1116 | B | C |
| 1117 | B | B |
| 1118 | A | B |
| 1119 | B | B |
| 1120 | B | B |
| 1121 | C | C |
| 1122 | B | C |
| 1123 | C | B |
| 1124 | B | A |
| 1125 | A | B |
| 1126 | B | B |
| 1127 | B | B |

### MSD-pSTAT-PBMC Assay

### Materials

Cryopreserved Peripheral Blood Mononuclear Cells (PBMC) are from AllCells. Recombinant Human IL-4 and IL-6 are from Peprotech. Mouse monoclonal anti-STAT3 antibody, rabbit monoclonal anti-pY705-STAT3 antibody, rabbit monoclonal Anti-pY641-STAT6 antibody, and lysis buffer are from Cell Signaling Technology (CST). Mouse monoclonal anti-STAT6 antibody is from BioLegend. Assay plates, blocker, and anti-rabbit secondary antibody are from Meso Scale Discovery (MSD).

### Assay Method:

Cyropreserved PBMCs were thawed out and allowed to recover overnight in IMDM+10% heat-inactivated FBS prior to plating 50,000 (STAT3) or 25,000 (STAT6) cells per well in 96-well U-bottom tissue culture plates. Cells were treated with compound for 3 hrs, then stimulated with 10 ng/mL IL-6 (STAT3) or 1 ng/mL IL-4 (STAT6) for 10 min. Cells were then spun down and washed with ice-cold PBS prior to lysing the cell pellet with 1× lysis buffer (CST) with 1× HALT protease and phosphatase inhibitor cocktail (Thermo). Lysates were transferred to and incubated overnight at 4° C. with shaking in QuickPlex 96-well high bind assay plates (MSD) pre-coated overnight with 30 μL per well of 0.6 μg/mL mouse monoclonal anti-STAT3 antibody (STAT3) or 2 μg/mL mouse monoclonal anti-STAT6 antibody (STAT6) in 1× PBS, and pre-blocked for 1 hour with 3% Blocker-A (MSD). Captured protein in the assay plates were then washed and probed with 25 μL per well of 0.25 Sg/ml rabbit monoclonal anti-pY705-STAT3 antibody (STAT3) or 0.18 μg/ml rabbit monoclonal Anti-pY641-STAT6 antibody (STAT6) in 1% Blocker-A for 1 hour at room temperature with shaking, then washed and probed with 25 ul per well of 1 ug/ml Sulfo-TAG Labeled Goat Anti-Rabbit Antibody (MSD) in 1% Blocker-A for 1 hour at room temperature with shaking. Assay plates were then washed and 150 μL of 1× Read Buffer-T (MSD) was added to each well prior to reading on an SQ120 MSD Plate Reader. Assay signal from each sample was subtracted by the signal from unstimulated control wells and normalized to DMSO control wells. IC₅₀ values were calculated using Graph Pad Prism Dose-Response Nonlinear Regression with variable slope. Results are shown in Table 114. For both pSTAT3 and pSTAT6 A=≤100 nM; B 100-1000 nM; C=>1000-3000 nM; D=>3 μM; and NT=Not Tested.

TABLE 1114

| Cmp# | pSTAT3 | pSTAT6 |
|---|---|---|
| 125 | D | C |
| 126 | B | A |
| 127 | C | B |
| 128 | D | B |
| 129 | D | B |
| 130 | A | D |
| 131 | B | A |
| 132 | A | D |
| 133 | D | B |
| 134 | D | B |
| 135 | D | B |
| 136 | A | C |
| 137 | D | B |
| 138 | D | B |
| 139 | B | A |
| 140 | A | C |
| 141 | B | B |
| 142 | B | A |
| 143 | B | A |
| 144 | C | B |
| 145 | A | A |
| 146 | C | A |
| 147 | B | A |
| 148 | D | B |
| 149 | D | D |
| 150 | B | D |
| 151 | B | B |
| 152 | C | B |
| 153 | D | D |
| 154 | B | B |
| 155 | B | A |
| 156 | B | A |
| 157 | B | B |
| 158 | A | D |
| 159 | D | D |
| 160 | A | A |
| 161 | A | D |
| 162 | B | B |
| 163 | D | A |
| 164 | B | B |
| 165 | C | B |
| 166 | B | A |
| 167 | D | B |
| 168 | B | A |
| 169 | B | A |
| 170 | B | A |
| 171 | D | D |
| 172 | A | C |
| 173 | B | D |
| 174 | A | B |
| 175 | A | B |
| 176 | A | C |
| 177 | A | C |
| 178 | B | D |
| 179 | A | C |
| 180 | A | D |
| 181 | A | D |
| 182 | A | C |
| 183 | B | A |
| 184 | B | A |
| 185 | B | A |
| 186 | A | D |
| 187 | A | C |
| 188 | A | B |
| 189 | A | D |
| 190 | A | A |
| 191 | A | B |
| 192 | B | C |
| 193 | A | A |
| 194 | B | A |
| 195 | B | D |
| 196 | B | D |
| 197 | D | D |
| 198 | D | D |
| 199 | D | D |
| 200 | C | D |
| 201 | B | D |
| 202 | D | C |

TABLE 115

| Cmp # | STAT3 | STAT6 |
|---|---|---|
| 248 | D | NT |
| 249 | A | B |
| 250 | A | NT |
| 251 | A | NT |
| 252 | C | B |
| 253 | D | B |
| 254 | B | NT |
| 255 | A | D |
| 256 | A | NT |
| 257 | A | NT |
| 258 | A | NT |
| 259 | A | NT |
| 260 | C | NT |
| 261 | A | NT |
| 262 | A | D |
| 263 | A | D |
| 264 | B | NT |
| 265 | B | NT |
| 266 | A | NT |
| 267 | A | NT |
| 268 | B | NT |
| 269 | NT | A |
| 270 | NT | B |
| 271 | B | NT |
| 272 | A | NT |
| 273 | B | NT |
| 274 | A | NT |
| 275 | D | NT |
| 276 | B | NT |
| 277 | A | NT |
| 278 | B | NT |
| 279 | A | NT |
| 280 | A | D |
| 281 | A | NT |
| 282 | A | NT |
| 283 | A | NT |
| 284 | A | D |
| 285 | D | NT |
| 286 | A | NT |
| 287 | A | NT |
| 288 | B | NT |
| 289 | A | NT |
| 290 | B | NT |
| 291 | A | NT |
| 292 | A | D |
| 293 | A | NT |
| 294 | C | NT |
| 295 | C | NT |
| 296 | A | NT |
| 297 | B | NT |
| 298 | B | NT |
| 299 | NT | B |
| 300 | A | NT |
| 301 | A | NT |
| 302 | A | C |
| 303 | NT | B |
| 304 | A | D |
| 305 | NT | B |
| 306 | C | NT |
| 307 | D | NT |
| 308 | D | NT |
| 309 | D | NT |
| 310 | B | NT |
| 311 | D | NT |
| 312 | NT | D |
| 313 | C | NT |
| 314 | NT | D |
| 315 | D | A |
| 316 | NT | A |
| 317 | NT | A |
| 318 | NT | D |
| 319 | NT | C |
| 320 | A | B |
| 321 | NT | A |
| 322 | NT | A |
| 323 | D | NT |
| 324 | A | D |
| 325 | D | A |
| 326 | D | A |
| 327 | NT | D |
| 328 | NT | B |
| 329 | NT | D |
| 330 | NT | D |
| 331 | NT | C |
| 332 | NT | D |
| 333 | NT | D |
| 334 | NT | A |
| 335 | NT | A |
| 336 | NT | A |
| 337 | D | A |
| 338 | NT | A |
| 339 | NT | A |
| 340 | NT | A |
| 341 | NT | A |
| 342 | NT | A |
| 343 | D | A |
| 344 | NT | A |
| 345 | NT | A |
| 346 | NT | A |
| 347 | NT | A |
| 348 | NT | A |
| 349 | NT | A |
| 350 | NT | A |
| 351 | NT | A |
| 352 | NT | A |
| 353 | NT | A |
| 354 | D | A |
| 355 | NT | A |
| 356 | NT | B |
| 357 | NT | A |
| 358 | NT | A |
| 359 | NT | A |
| 360 | NT | A |
| 361 | NT | A |
| 362 | NT | A |
| 363 | NT | A |
| 364 | NT | A |
| 365 | NT | A |
| 366 | NT | B |
| 367 | NT | B |
| 368 | NT | D |
| 369 | NT | C |
| 370 | NT | A |
| 371 | NT | C |
| 372 | NT | D |
| 373 | NT | D |
| 374 | NT | A |
| 375 | NT | A |
| 376 | NT | A |
| 377 | NT | A |
| 378 | NT | A |
| 379 | NT | A |
| 380 | D | A |
| 381 | D | A |
| 382 | D | A |
| 384 | NT | A |
| 385 | NT | A |
| 386 | NT | A |
| 387 | NT | A |
| 388 | D | A |
| 389 | B | NT |
| 390 | B | NT |
| 391 | C | NT |
| 392 | B | NT |
| 393 | B | NT |
| 394 | A | NT |
| 395 | B | NT |
| 396 | D | C |
| 397 | D | D |
| 398 | D | NT |
| 399 | C | NT |
| 400 | B | NT |
| 401 | D | NT |
| 402 | D | C |
| 403 | D | B |
| 404 | D | C |
| 405 | D | C |
| 406 | D | C |
| 407 | D | C |
| 408 | D | B |
| 409 | D | C |
| 410 | D | D |
| 411 | NT | A |
| 412 | NT | B |
| 413 | NT | B |
| 414 | NT | B |
| 415 | A | C |
| 416 | NT | D |
| 417 | NT | A |
| 418 | NT | A |
| 419 | D | B |
| 420 | B | NT |
| 421 | D | B |
| 422 | A | NT |
| 423 | NT | B |
| 424 | NT | B |
| 425 | NT | D |
| 426 | D | NT |
| 427 | NT | A |
| 428 | A | NT |
| 429 | NT | A |
| 430 | B | NT |
| 431 | B | NT |
| 432 | B | NT |
| 433 | C | NT |
| 434 | B | NT |
| 435 | B | NT |
| 436 | NT | D |
| 437 | B | NT |
| 438 | C | NT |
| 439 | B | NT |
| 440 | NT | A |
| 441 | NT | A |
| 442 | NT | A |
| 443 | NT | B |
| 444 | NT | B |
| 445 | A | NT |
| 446 | A | NT |
| 447 | A | NT |
| 448 | NT | A |
| 449 | NT | B |
| 450 | NT | B |
| 451 | NT | B |
| 452 | NT | A |
| 453 | NT | B |
| 454 | NT | D |
| 455 | NT | D |
| 456 | NT | A |
| 457 | D | A |
| 458 | NT | A |
| 459 | NT | A |
| 460 | NT | A |
| 461 | NT | A |
| 462 | NT | A |
| 463 | A | NT |
| 464 | A | NT |
| 465 | NT | D |
| 466 | NT | D |
| 467 | NT | D |
| 468 | NT | A |
| 469 | NT | B |
| 470 | NT | A |
| 471 | NT | A |
| 472 | NT | A |
| 473 | NT | D |
| 474 | NT | A |
| 475 | NT | B |
| 476 | NT | A |
| 477 | NT | A |
| 478 | NT | A |
| 479 | NT | A |
| 480 | NT | A |
| 481 | NT | A |
| 482 | NT | A |
| 483 | NT | A |
| 484 | A | NT |
| 485 | D | A |
| 486 | NT | A |
| 487 | NT | C |
| 488 | NT | C |
| 489 | NT | B |
| 490 | NT | B |
| 491 | NT | A |
| 492 | NT | A |
| 493 | A | NT |
| 494 | A | NT |
| 940 | A | B |
| 941 | A | C |
| 942 | B | D |
| 943 | A | D |
| 944 | B | NT |
| 945 | B | NT |
| 946 | B | NT |
| 947 | A | NT |
| 948 | C | NT |
| 949 | C | NT |
| 950 | A | NT |
| 951 | A | NT |
| 952 | B | NT |
| 953 | A | NT |
| 954 | A | D |
| 955 | A | NT |
| 956 | A | NT |
| 957 | D | NT |
| 958 | C | NT |
| 959 | A | NT |
| 960 | B | NT |
| 961 | A | C |
| 962 | A | D |
| 963 | B | NT |
| 964 | B | NT |
| 965 | B | NT |
| 966 | A | NT |
| 967 | A | NT |
| 968 | A | NT |
| 969 | D | NT |
| 970 | A | NT |
| 971 | C | NT |
| 972 | A | NT |
| 973 | A | D |
| 324 | A | D |
| 974 | B | NT |
| 975 | A | NT |
| 976 | NT | D |
| 977 | NT | B |
| 978 | B | NT |
| 979 | D | NT |
| 980 | C | NT |
| 981 | NT | D |
| 982 | NT | D |
| 983 | A | NT |
| 984 | A | D |
| 985 | D | NT |
| 986 | C | NT |
| 987 | B | NT |
| 988 | A | NT |
| 989 | NT | D |
| 990 | D | NT |
| 991 | D | NT |
| 992 | NT | B |
| 993 | A | NT |
| 994 | B | NT |
| 995 | A | NT |
| 996 | B | NT |
| 997 | A | NT |
| 998 | A | NT |
| 999 | A | NT |
| 1000 | A | NT |
| 1001 | A | NT |
| 1002 | A | NT |
| 1003 | C | NT |
| 1004 | B | NT |
| 1005 | A | NT |
| 1006 | A | NT |
| 1007 | D | B |
| 1008 | B | D |
| 1009 | A | B |
| 1010 | C | B |
| 1011 | A | B |
| 1012 | NT | A |
| 1013 | NT | B |
| 1014 | A | B |
| 1015 | A | NT |
| 1016 | B | NT |
| 1017 | A | NT |
| 1018 | A | NT |
| 1019 | A | NT |
| 1020 | A | NT |
| 1021 | A | NT |
| 1022 | A | NT |
| 1023 | B | NT |
| 1024 | B | NT |
| 1025 | A | NT |
| 1026 | A | NT |
| 1027 | A | NT |
| 1028 | B | NT |
| 1029 | A | NT |
| 1030 | D | NT |
| 1031 | A | NT |
| 1032 | A | NT |
| 1033 | A | NT |
| 1034 | A | NT |
| 1035 | A | NT |
| 1036 | A | NT |
| 1037 | A | NT |
| 1038 | A | NT |
| 1039 | A | NT |
| 1040 | A | NT |
| 1041 | A | NT |
| 1042 | B | NT |
| 1043 | A | NT |
| 1044 | A | NT |
| 1045 | A | D |
| 1046 | B | NT |
| 1047 | B | NT |
| 1048 | A | NT |
| 1049 | A | NT |
| 1050 | B | NT |
| 1051 | B | NT |
| 1052 | B | NT |
| 1053 | A | NT |
| 1054 | A | NT |
| 1055 | A | D |
| 1056 | A | NT |
| 1057 | A | NT |
| 1058 | A | NT |
| 1059 | A | NT |
| 1060 | B | NT |
| 1061 | B | NT |
| 1062 | B | NT |
| 1063 | A | NT |
| 1064 | A | NT |
| 1065 | A | NT |
| 1066 | B | NT |
| 1067 | B | NT |
| 1068 | A | NT |
| 1069 | A | NT |
| 1070 | A | NT |
| 1071 | A | NT |
| 1072 | A | NT |
| 1073 | B | NT |
| 1074 | A | NT |
| 1075 | A | NT |
| 1076 | A | NT |
| 1077 | A | NT |
| 1078 | A | NT |
| 1079 | A | NT |
| 1080 | NT | A |
| 1081 | NT | A |
| 1082 | D | B |
| 1083 | C | B |
| 1084 | B | NT |
| 1085 | B | A |
| 1086 | A | B |
| 1087 | B | A |
| 1088 | C | A |
| 1089 | A | B |
| 1090 | C | B |
| 1091 | D | D |
| 1092 | B | A |
| 1093 | D | B |
| 1094 | C | B |
| 1095 | A | NT |
| 1096 | A | NT |
| 1097 | C | B |
| 1098 | A | NT |
| 1099 | A | NT |
| 1100 | B | A |
| 1101 | D | A |
| 1102 | A | NT |
| 1105 | A | NT |
| 1106 | A | B |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the structural Formula I: 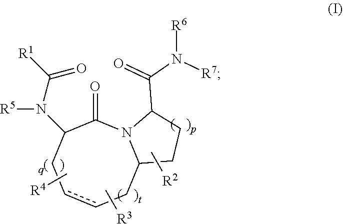
or a pharmaceutically acceptable salt thereof, wherein:
q is 0 or 1 and t is 0, 1, or 2, provided that at least one of q or t is 1;
p is 1 or 2;
the dotted line represents a single or double bond;
R¹ is selected from an 8- to 10-membered fused bicyclic heteroaryl substituted with —CR^{1a}R^{2a}P(O)OR^{1b}OR^{2b} or —CR^{1a}R^{2a}P(O)[OR^{1b}][NH(AA)C(O)OR^{T}], an 8- to 10-membered fused bicyclic heterocyclyl substituted with —CR^{1a}R^{2a}P(O)OR^{1b}OR^{2b} or —CR^{1a}R^{2a}P(O)[OR^{1b}][NH(AA)C(O)OR^{T}], an aryl substituted with CR^{1a}R^{2a}P(O)OR^{1b}OR^{2b} or —CR^{1a}R^{2a}P(O)[OR^{1b}][NH(AA)C(O)OR^{T}], a —(C₁-C₄)alkyl(aryl) wherein said aryl portion of —(C₁-C₄)alkyl(aryl) is substituted with —CR^{1a}R^{2a}P(O)OR^{1b}OR^{2b} or —CR^{1a}R^{2a}P(O)[OR^{1b}][NH(AA)C(O)OR^{T}], and a —(C₂-C₄)alkenyl(aryl) wherein said aryl portion of —(C₂-C₄)alkenyl(aryl) is substituted with —CR^{1a}R^{2a}P(O)OR^{1b}OR^{2b} or —CR^{1a}R^{2a}P(O)[OR^{1b}][NH(AA)C(O)OR^{T}];
R^{1a} and R^{2a} are each absent or are independently selected from hydrogen, cyano, (C₁-C₄)alkyl, hydroxy(C₁-C₄)alkyl and fluoro; or R^{1a} and R^{2a} taken together with the carbon they are attached form oxo;
R^{1b} and R^{2b} are each absent or are independently selected from hydrogen, (C₁-C₄)alkyl, halo(C₁-C₄)alkyl, —[(C₁-C₄)alkyl]-OC(O)—[(C₁-C₄)alkyl], —[(C₁-C₄)alkyl]-C(O)O—[(C₁-C₄)alkyl], —[(C₁-C₄)alkyl]-O—[(C₁-C₂₀)alkyl], —[(C₁-C₄)alkyl]-OC(O)-[halo(C₁-C₄)alkyl], [(C₁-C₄)alkyl]—OC(O)O-[5- to 7-membered heterocyclyl], [(C₁-C₄)alkyl]-OC(O)-[5- to 7-membered heterocyclyl], —[(C₁-C₄)alkyl]-OC(O)—[(C₁-C₄)alkyl]-OH, —[(C₁-C₄)alkyl]-OC(O)—[(C₁-C₄)alkyl]-O—[(C₁-C₄)alkyl], —[(C₁-C₄)alkyl]-OC(O)O—[(C₁-C₄)alkyl], —[(C₁-C₄)alkyl]-OC(O)O-[halo(C₁-C₄)alkyl], —[(C₁-C₄)alkyl]-OC(O)O—[(C₁-C₄)alkyl]-OH, —[(C₁-C₄)alkyl]-OC(O)O—[(C₁-C₄)alkyl]-O—[(C₁-C₄)alkyl], —[(C₁-C₄)alkyl]-SC(O)—[(C₁-C₄)alkyl], —[(C₁-C₄)alkyl]-SC(O)-[halo(C₁-C₄)alkyl], —[(C₁-C₄)alkyl]-SC(O)—[(C₁-C₄)alkyl]-OH, —[(C₁-C₄)alkyl]-SC(O)—[(C₁-C₄)alkyl]-O—[(C₁-C₄)alkyl], —[(C₁-C₄)alkyl]-OC(O)NH(C₁-C₄)alkyl], —[(C₁-C₄)alkyl]-OC(O)N[(C₁-C₄)alkyl]₂, 5- to 6-membered heteroaryl, and aryl, wherein said 5- to 6-membered heteroaryl and aryl are each optionally and independently substituted with, as valency permits, 1 to 2 groups selected from halo, cyano, and (C₁-C₄)alkyl and wherein said 5- to 7-membered heterocyclyl of [(C₁-C₄)alkyl]-OC(O)O-[5- to 7-membered heterocyclyl] and [(C₁-C₄)alkyl]-OC(O)-[5- to 7-membered heterocyclyl] are each optionally and independently substituted with, as valency permits 1 to 2 groups selected from C(O)OR^{h};
R² is selected from hydrogen, halo, (C₁-C₄)alkyl, halo(C₁-C₄)alkyl, (C₁-C₄)alkoxy, halo(C₁-C₄)alkoxy, hydroxy(C₁-C₄)alkyl, cyano, and hydroxyl;
R³ and R⁴ are each independently selected from hydrogen, halo, (C₁-C₄)alkyl, halo(C₁-C₄)alkyl, hydroxy(C₁-C₄)alkyl, —(C₁-C₄)alkylphenyl, (C₁-C₄)alkoxy, halo(C₁-C₄)alkoxy, —(C₁-C₄)alkyl(C₁-C₄)alkoxy, hydroxyl, cyano, —NR^{a}R^{b}, phenyl, (C₃-C₆)cycloalkyl, 5- to 6-membered heteroaryl, and 4- to 6-membered heterocyclyl, wherein said phenyl, (C₃-C₆)cycloalkyl, 5- to 6-membered heteroaryl, and 4- to 6-membered heterocyclyl are each optionally substituted with, as valency permits, 1 to 3 groups selected from R^{S};
or R³ and R⁴ are taken together on the same carbon atom to form a (C₃-C₆)cycloalkyl or a 4- to 6-membered heterocyclyl each optionally substituted with, as valency permits, 1 to 3 groups selected from halo, (C₁-C₄)alkyl, halo(C₁-C₄)alkyl, (C₁-C₄)alkoxy, and halo(C₁-C₄)alkoxy;
R⁵ and R⁶ are each independently selected from hydrogen and (C₁-C₄)alkyl;
R⁷ is selected from (C₁-C₄)alkyl, phenyl, 4- to 9-membered monocyclic or bicyclic heterocyclyl, and 5- to 10-membered monocyclic or bicyclic heteroaryl, wherein said (C₁-C₄)alkyl is optionally substituted with, as valency permits, 1 to 3 groups selected from R^{Y} and said phenyl, 4- to 9-membered monocyclic or bicyclic heterocyclyl, and 5- to 10-membered monocyclic or bicyclic heteroaryl are each optionally substituted with, as valency permits, 1 to 3 groups selected from R^{Z}; or
R⁶ and R⁷ together with the nitrogen atom to which they are attached form a 4- to 14-membered monocyclic or bicyclic heterocyclyl or a 5- to 12-membered monocyclic or bicyclic heteroaryl, each of which being optionally substituted with, as valency permits, 1 to 3 groups selected from R^{Q};
AA is the residue of an alpha or beta natural or non-natural amino acid;
R^{T} is selected from (C₁-C₄)alkyl, benzyl, and phenyl, wherein said phenyl is optionally substituted with 1 or 2 groups selected from halo, (C₁-C₄)alkyl, and halo(C₁-C₄)alkyl;
R^{Q} is selected from halo, (C₂-C₄)alkenyl, (C₁-C₄)alkyl, (C₁-C₄)alkoxy, halo(C₁-C₄)alkoxy, cyano, phenyl, hydroxyl, 4- to 6-membered heterocyclyl, 5- to 10-membered monocyclic or bicyclic heteroaryl, (C₃-C₆)cycloalkyl, oxo, imino, —O(phenyl), —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)═NH(C₁-C₄)alkyl, —S(O)NR^{e}R^{f}, and —S(O)₂NR^{e}R^{f}, wherein said (C₂-C₄)alkenyl and (C₁-C₄)alkyl are each optionally and independently substituted with, as valency permits, 1 to 3 groups selected from R^{M}, and wherein said phenyl, 5- to 10-membered monocyclic or bicyclic heteroaryl, (C₃-C₆)cycloalkyl, and 4- to 6-membered heterocyclyl are each optionally and independently substituted with, as valency permits, 1 to 3 groups selected from R^{F};
R^{Y} is selected from halo, (C₁-C₄)alkoxy, halo(C₁-C₄)alkoxy, cyano, —C(O)R^{g}, —C(O)OR^{e}, —NHC(O)R^{e}, —NR^{a}R^{b}, —S(O)R^{e}R^{f}, —S(O)₂R^{f}, —S(O)NR^{e}R^{f}, —S(O)=NH(C₁-C₄)alkyd, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from RN;
R^{J} and R^{M} are each independently selected from halo, (C₁-C₄)alkoxy, halo(C₁-C₄)alkoxy, cyano, —C(O)R^{g}, —C(O)OR^{e}, —NHC(O)R^{e}, —C(O)NR^{c}R^{d}, —NR^{a}R^{b}, —S(O)R^{e}R^{f}, —S(O)₂R^{f}, —S(O)NR^{e}R^{f}, —S(O)═NH(C₁-C₄)alkyl, —S(O)₂NR^{e}R^{f}, hydroxyl, phenyl, 4- to 6-membered heterocyclyl, and 5- to 10-membered monocyclic or bicyclic heteroaryl, wherein said phenyl, 4- to 6-membered heterocyclyl, and 5- to 10-membered monocyclic or bicyclic heteroaryl are each optionally substituted with, as valency permits, 1 to 3 groups selected from R^{X};
R^{F}, R^{S}, R^{X}, and R^{Z} are each independently selected from halo, cyano, (C₁-C₄)alkyl, halo(C₁-C₄)alkyl, —(C₁-C₄)alkyl(C₁-C₄)alkoxy, hydroxy(C₁-C₄)alkyl, —(C₁-C₄)alkylphenyl, (C₂-C₄)alkenyl, halo(C₂-C₄)alkenyl, (C₂-C₄)alkynyl, halo(C₂-C₄)alkynyl, (C₁-C₄)alkoxy, halo(C₁-C₄)alkoxy, hydroxyl, oxo, imino, phenyl, —S(O)R^{e}R^{f}, —S(O)₂R^{f}, —S(O)═NH(C₁-C₄)alkyl, —S(O)NR^{e}R^{f}, and —S(O)₂NR^{e}R^{f}, —C(O)OR^{e}, —NR^{c}C(O)R^{e}, —C(O)R^{g}, —C(O)NR^{c}R^{d}, and —NR^{a}R^{b}, wherein said phenyl and said phenyl for the group —(C₁-C₄)alkylphenyl are each optionally and independently substituted with, as valency permits 1 to 3 groups selected from halo, cyano, (C₁-C₁₀)alkyl, (C₂-C₁₀)alkenyl, (C₂-C₁₀)alkynyl, halo(C₁-C₁₀)alkyl, (C₁-C₁₀)alkoxy, and halo(C₁-C₁₀)alkoxy, wherein said (C₁-C₁₀)alkyl, (C₂-C₁₀)alkenyl and (C₂-C₁₀)alkynyl are each optionally substituted with, as valency permits a 5- to 10-membered monocyclic or bicyclic heteroaryl or a 4- to 10-membered monocyclic or bicyclic heterocyclyl each of said 5- to 10-membered monocyclic and bicyclic heteroaryl or a 4- to 10-membered monocyclic or bicyclic heterocyclyl being optionally substituted with oxo or a 5- to 7-membered heterocyclyl that is optionally substituted with 1 to 2 oxo; and
R^{a}, R^{b}, R^{c}, R^{d}, R^{e}, R^{f}, R^{g}, and R^{h} are each independently selected from, as valency permits, hydrogen, (C₁-C₄)alkyl, phenyl, (C₃-C₆)cycloalkyl, 4- to 6-membered heterocyclyl and 5- to 6-membered heteroaryl, wherein said (C₁-C₄)alkyl is optionally substituted with, as valency permits, 1 to 3 groups selected from R^{J}, and said phenyl, (C₃-C₆)cycloalkyl, 4- to 6-membered heterocyclyl, and 5- to 6-membered heteroaryl are each independentlsy optionally substituted with, as valency permits, 1 to 3 groups selected from halo, cyano, (C₁-C₄)alkyl, halo(C₁-C₄)alkyl, (C₁-C₄)alkoxy, halo(C₁-C₄)alkoxy, hydroxyl, phenyl, and benzyl.

2. The compound of claim 1, wherein the compound is of the structural Formula II: 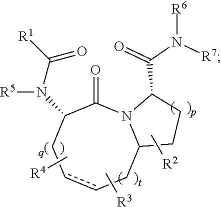
or a pharmaceutically acceptable salt thereof.

3. The compound of claim 1, wherein the compound is of the structural III, IV, V, or VII: 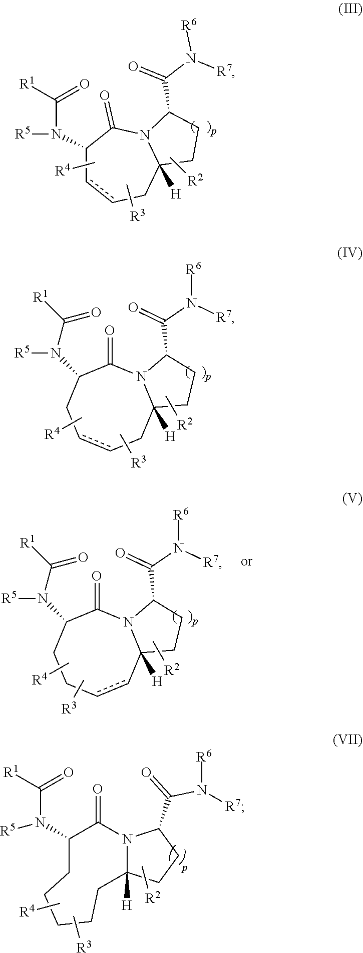
or a pharmaceutically acceptable salt thereof.

4. The compound of claim 1, wherein the compound is of the structural VIII, VIII′, IX, X, XI, XII, or XIII: 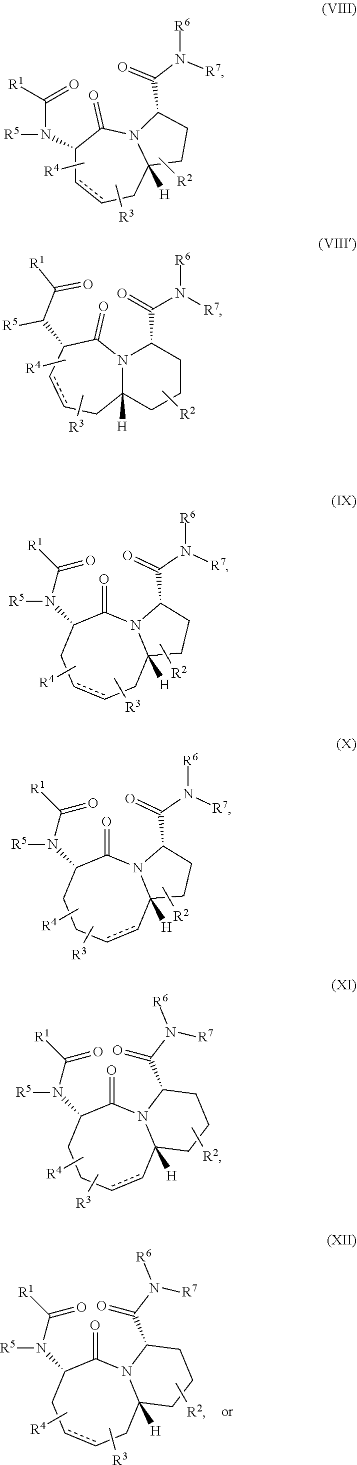 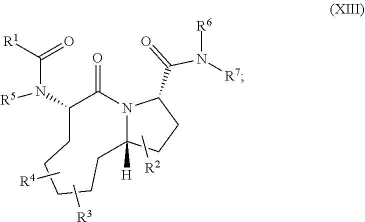
or a pharmaceutically acceptable salt thereof.

5. The compound of claim 1, wherein the compound is of the structural XIV, XV, XVI, XVI′, XVII, XVIII, XX, XXI, XXII, XXIII, XXIV, XXV, XXVI, XXVII, or XXVIII: 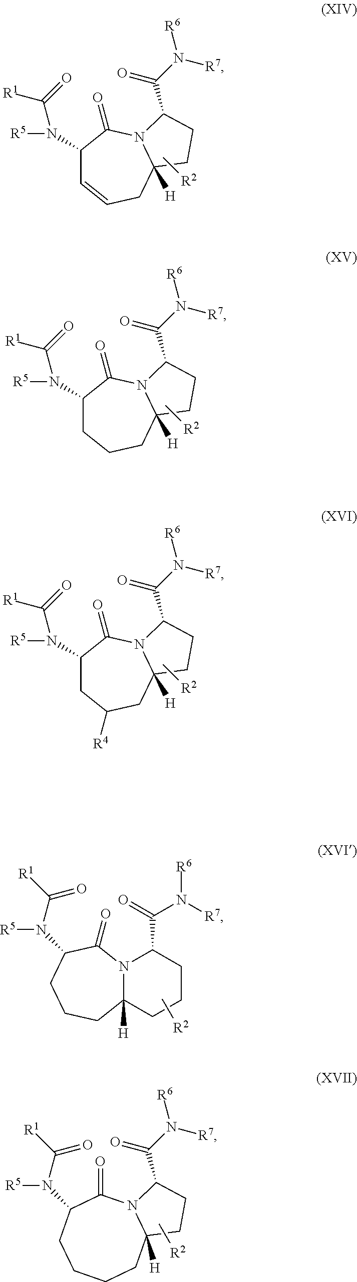 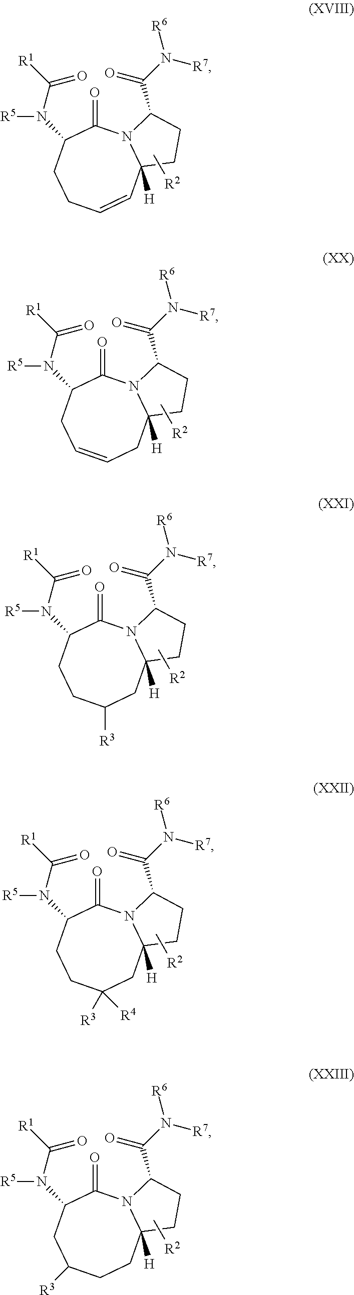 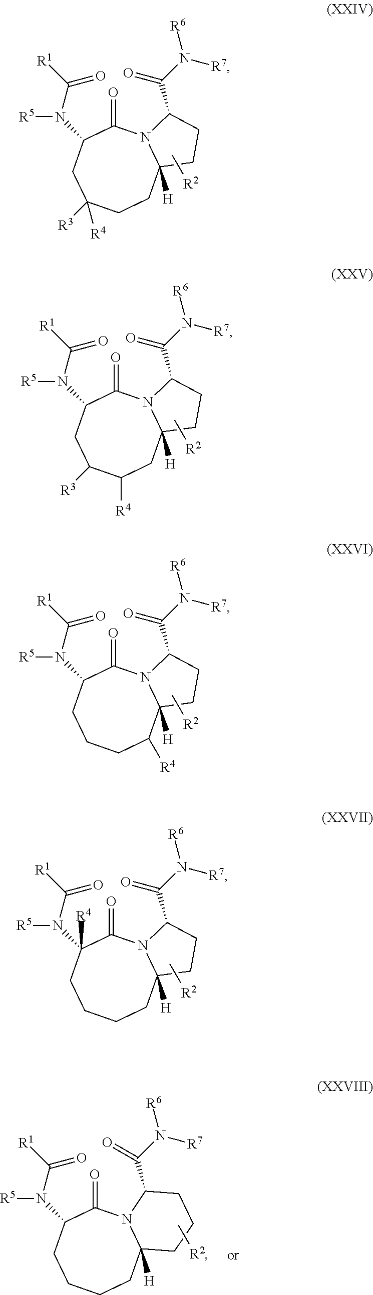 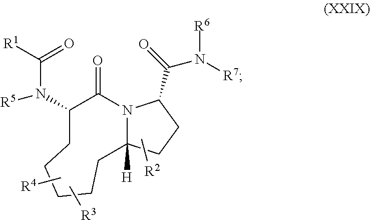
or a pharmaceutically acceptable salt thereof.

6. The compound of claim 1, wherein the compound is of the structural Formula XXX, XXXI, XXXII, XXXIII XXXIV, XXXV, XXXVI, or XXVII: 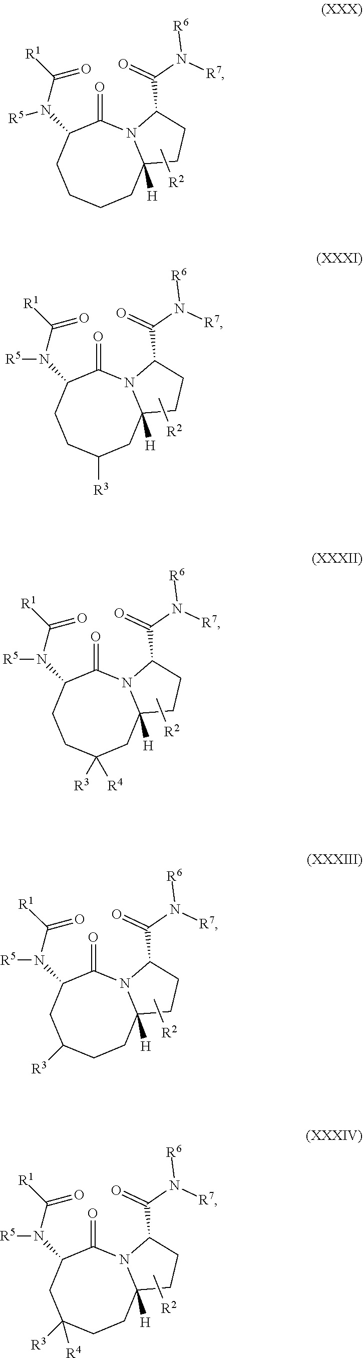 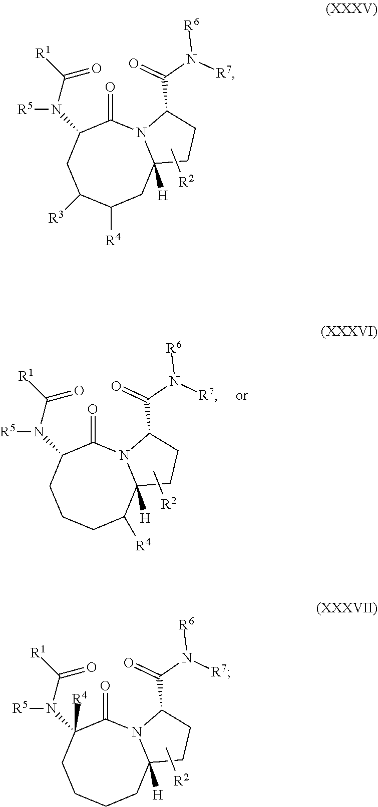
or a pharmaceutically acceptable salt thereof.

7. The compound of claim 1, or a pharmaceutically acceptable salt thereof, wherein R³ is selected from hydrogen, (C₁-C₄)alkyl, hydroxyl, (C₁-C₄)alkoxy, —(C₁-C₄)alkylphenyl, and 4- to 6-membered heterocyclyl; or R³ and R⁴ are taken together on the same carbon atom to form a (C₃-C₆)cycloalkyl.

8. The compound of claim 1, or a pharmaceutically acceptable salt thereof, wherein R⁴ is selected from hydrogen (C₁-C₄)alkyl, and hydroxyl; or R³ and R⁴ are taken together on the same carbon atom to form a (C₃-C₆)cycloalkyl.

9. The compound of claim 1, or a pharmaceutically acceptable salt thereof, wherein R³ is selected from hydrogen, (C₁-C₂)alkyl, hydroxyl, (C₁-C₂)alkoxy, benzyl, and azetidinyl, or R³ and R⁴ are taken together on the same carbon atom to form a cyclopropyl.

10. The compound of claim 1, or a pharmaceutically acceptable salt thereof, wherein R³ and R⁴ are hydrogen.

11. The compound of claim 1, or a pharmaceutically acceptable salt thereof, wherein R² is selected from hydrogen and hydroxyl.

12. (canceled)

13. The compound of claim 1, or a pharmaceutically acceptable salt thereof, wherein R⁵ is hydrogen.

14. The compound of claim 1, or a pharmaceutically acceptable salt thereof, wherein R¹ is selected from 8- to 10-membered fused bicyclic heteroaryl and aryl, each of which are substituted with —(CR^{1a}R^{2a}P(O)OR^{1b}OR^{2b} or —CR^{1a}R^{2a}P(O)[OR^{1b}][NH(AA)C(O)OR^{T}].

15. The compound of claim 1, or a pharmaceutically acceptable salt thereof, wherein, R¹ is selected from benzothiophenyl, indolyl, and naphthalenyl, each of which are substituted with —CR^{1a}R^{2a}P(O)OR^{1b}OR^{2b} or —CR^{1a}R^{2a}P(O)[OR^{1b}][NH(AA)C(O)OR^{T}].

16. The compound of claim 1, or a pharmaceutically acceptable salt thereof, wherein, R¹ is selected from

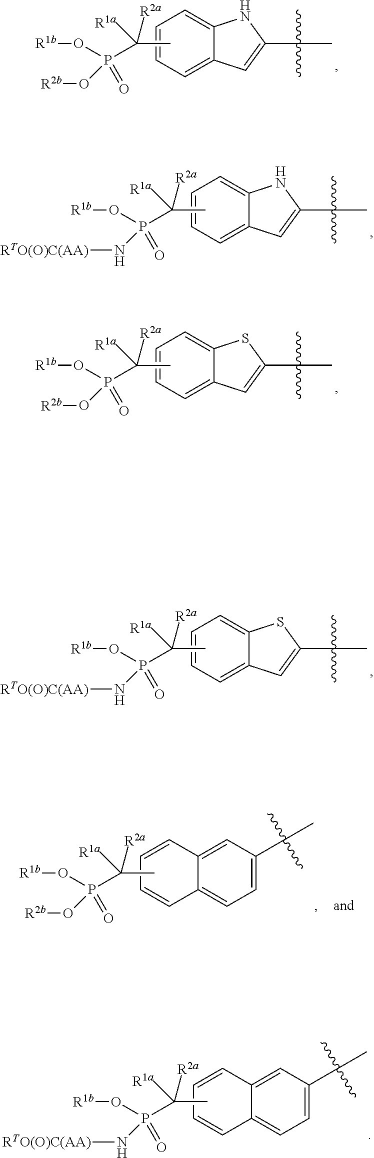

17. The compound of claim 1, or a pharmaceutically acceptable salt thereof, wherein, R¹ is

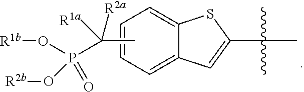

18. The compound of claim 1, or a pharmaceutically acceptable salt thereof, wherein R^{1a} is hydrogen and R^{2a} is fluoro or R^{1a} is fluoro and R^{2a} is fluoro.

19.-45. (canceled)

46. A pharmaceutically acceptable composition comprising the compound of claim 1, or a pharmaceutically acceptable salt thereof; and a pharmaceutically acceptable carrier.

47. A method of treating a condition responsive to the modulation of STAT3 or STAT6 in a subject comprising administering to the subject a therapeutically effective amount of the compound of claim 1.
